(19) United States
(12) Patent Application Publication
(10) Pub. No.: US 2006/0084054 A1
(43) Pub. Date: Apr. 20, 2006

(54) Novel proteins and nucleic acids encoding same

(76) Inventors: John Alsobrook, Madison, CT (US); David Anderson, Plantsville, CT (US); Jason Baumgartner, New Haven, CT (US); Constance Berghs, New Haven, CT (US); Ferenc Boldog, North Haven, CT (US); Catherine Burgess, Wethersfield, CT (US); Stacie Casman, North Haven, CT (US); Elina Catterton, Milford, CT (US); Mohanraj Dhanabal, Branford, CT (US); Shlomit Edinger, New Haven, CT (US); Karen Ellerman, Branford, CT (US); Seth Ettenberg, New Haven, CT (US); Esha Gangolli, Acton, MA (US); Valerie Gerlach, Branford, CT (US); Linda Gorman, Branford, CT (US); William Grosse, Branford, CT (US); Erik Gunther, Branford, CT (US); Xiaojia (Sasha) Guo, Branford, CT (US); Vladimir Gusev, Madison, CT (US); John Herrmann, Guilford, CT (US); Weizhen Ji, Branford, CT (US); Ramesh Kekuda, Stamford, CT (US); Nikolai Khramtsov, Branford, CT (US); William LaRochelle, Madison, CT (US); Li Li, Branford, CT (US); Hongping Liang, Hamden, CT (US); Kenneth Low, New Haven, CT (US); John MacDougall, Hamden, CT (US); Timothy Maclachlan, Unionville, CT (US); Uriel Malyankar, North Branford, CT (US); Kelly McQueeney, Ansonia, CT (US); Amanda Mezick, Hamden, CT (US); Charles Miller, Guilford, CT (US); Isabelle Millet, Milford, CT (US); Muralidhara Padigaru, Branford, CT (US); Meera Patturajan, Branford, CT (US); John Peyman, New Haven, CT (US); Xiaozhong Qian, Branford, CT (US); Luca Rastelli, Guilford, CT (US); Daniel Rieger, Branford, CT (US); Mark Rothenberg, Clinton, CT (US); Suresh Shenoy, Branford, CT (US); Richard Shimkets, Guilford, CT (US); Glennda Smithson, Guilford, CT (US); Kimberly Spytek, Ellington, CT (US); David Stone, Guilford, CT (US); Sujatha Sukumaran, Branford, CT (US); Edward S. Szekeres JR., Walling Ford, CT (US); Corine Vernet, Chernex (CH); Edward Voss, Wallingford, CT (US); Adam R. Wolenc, New Haven, CT (US); Mei Zhong, Branford, CT (US); Haihong Zhong, Guilford, CT (US)

Correspondence Address: Jenell Lawson;Intellectual Property, CURAGEN CORPORATION, 555 Long Wharf Drive, New Haven, CT, 06551, US

(21) Appl. No.: 10/455,772

(22) Filed: Jun. 4, 2003

Related U.S. Application Data
(63) Continuation-in-part of application No. 09/669,360, filed on Sep. 26, 2000, now abandoned.
(60) Provisional application No. 60/385,615, filed on Jun. 4, 2002.
(60) Provisional application No. 60/402,268, filed on Aug. 9, 2002.
(60) Provisional application No. 60/387,606, filed on Jun. 11, 2002.
(60) Provisional application No. 60/386,357, filed on Jun. 6, 2002.
(60) Provisional application No. 60/385,755, filed on Jun. 4, 2002.
(60) Provisional application No. 60/386,355, filed on Jun. 6, 2002.
(60) Provisional application No. 60/385,490, filed on Jun. 4, 2002.
(60) Provisional application No. 60/420,718, filed on Oct. 23, 2002.
(60) Provisional application No. 60/386,447, filed on Jun. 6, 2002.
(60) Provisional application No. 60/386,465, filed on Jun. 6, 2002.
(60) Provisional application No. 60/420,627, filed on Oct. 23, 2002.
(60) Provisional application No. 60/386,459, filed on Jun. 6, 2002.
(60) Provisional application No. 60/410,505, filed on Sep. 13, 2002.
(60) Provisional application No. 60/420,852, filed on Oct. 24, 2002.
(60) Provisional application No. 60/386,796, filed on Jun. 7, 2002.
(60) Provisional application No. 60/387,078, filed on Jun. 7, 2002.
(60) Provisional application No. 60/387,083, filed on Jun. 7, 2002.
(60) Provisional application No. 60/387,081, filed on Jun. 7, 2002.
(60) Provisional application No. 60/386,041, filed on Jun. 5, 2002.
(60) Provisional application No. 60/386,701, filed on Jun. 7, 2002.
(60) Provisional application No. 60/387,610, filed on Jun. 11, 2002.
(60) Provisional application No. 60/387,540, filed on Jun. 10, 2002.
(60) Provisional application No. 60/387,429, filed on Jun. 10, 2002.
(60) Provisional application No. 60/410,085, filed on Sep. 12, 2002.
(60) Provisional application No. 60/389,120, filed on Jun. 14, 2002.
(60) Provisional application No. 60/386,931, filed on Jun. 7, 2002.
(60) Provisional application No. 60/387,866, filed on Jun. 10, 2002.
(60) Provisional application No. 60/387,859, filed on Jun. 11, 2002.
(60) Provisional application No. 60/387,659, filed on Jun. 11, 2002.
(60) Provisional application No. 60/387,934, filed on Jun. 12, 2002.
(60) Provisional application No. 60/387,696, filed on Jun. 11, 2002.
(60) Provisional application No. 60/390,006, filed on Jun. 19, 2002.
(60) Provisional application No. 60/389,604, filed on Jun. 18, 2002.
(60) Provisional application No. 60/387,668, filed on Jun. 11, 2002.
(60) Provisional application No. 60/386,864, filed on Jun. 6, 2002.
(60) Provisional application No. 60/401,628, filed on Aug. 6, 2002.
(60) Provisional application No. 60/406,182, filed on Aug. 26, 2002.
(60) Provisional application No. 60/412,955, filed on Sep. 23, 2002.
(60) Provisional application No. 60/415,195, filed on Sep. 30, 2002.
(60) Provisional application No. 60/422,750, filed on Oct. 31, 2002.
(60) Provisional application No. 60/390,144, filed on Jun. 19, 2002.
(60) Provisional application No. 60/388,022, filed on Jun. 12, 2002.
(60) Provisional application No. 60/402,822, filed on Aug. 12, 2002.
(60) Provisional application No. 60/388,096, filed on Jun. 12, 2002.
(60) Provisional application No. 60/389,123, filed on Jun. 13, 2002.
(60) Provisional application No. 60/390,209, filed on Jun. 19, 2002.
(60) Provisional application No. 60/388,479, filed on Jun. 12, 2002.
(60) Provisional application No. 60/403,458, filed on Aug. 13, 2002.
(60) Provisional application No. 60/389,884, filed on Jun. 18, 2002.
(60) Provisional application No. 60/389,146, filed on Jun. 14, 2002.
(60) Provisional application No. 60/387,960, filed on Jun. 12, 2002.
(60) Provisional application No. 60/388,432, filed on Jun. 12, 2002.
(60) Provisional application No. 60/403,617, filed on Aug. 15, 2002.
(60) Provisional application No. 60/423,095, filed on Nov. 1, 2002.
(60) Provisional application No. 60/423,748, filed on Nov. 5, 2002.
(60) Provisional application No. 60/391,726, filed on Jun. 25, 2002.
(60) Provisional application No. 60/403,732, filed on Aug. 15, 2002.
(60) Provisional application No. 60/389,742, filed on Jun. 17, 2002.
(60) Provisional application No. 60/156,217, filed on Sep. 27, 1999.

(51) Int. Cl. C12Q 1/68; C07H 21/04; C12N 9/00

(52) U.S. Cl. 435/6; 435/69.1; 435/183; 435/320.1; 435/325; 530/350; 536/23.2

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

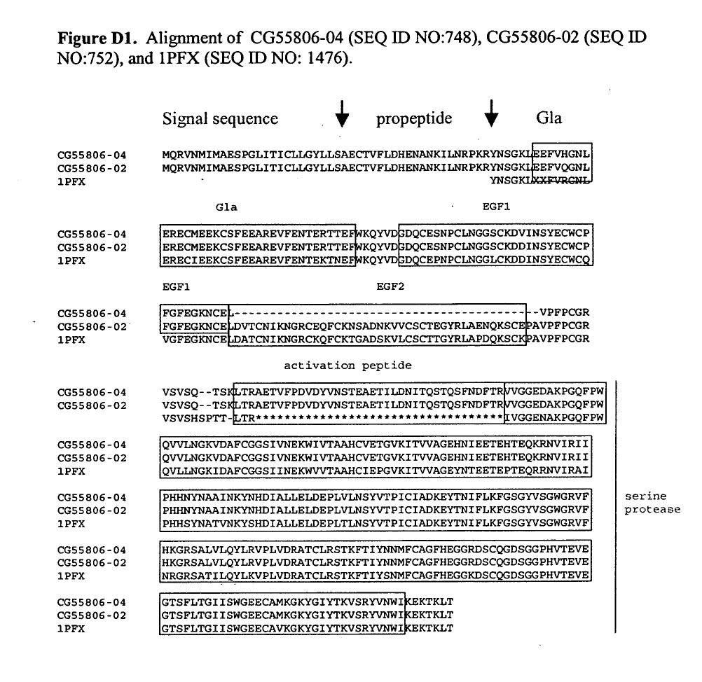

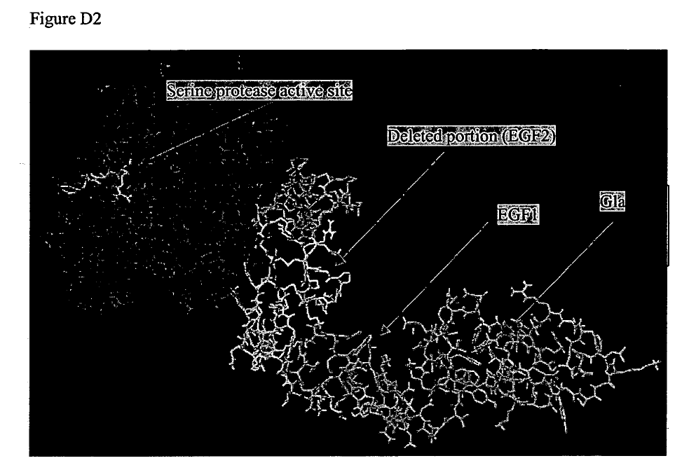

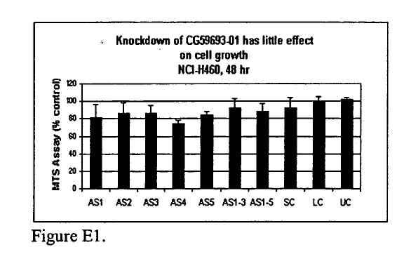

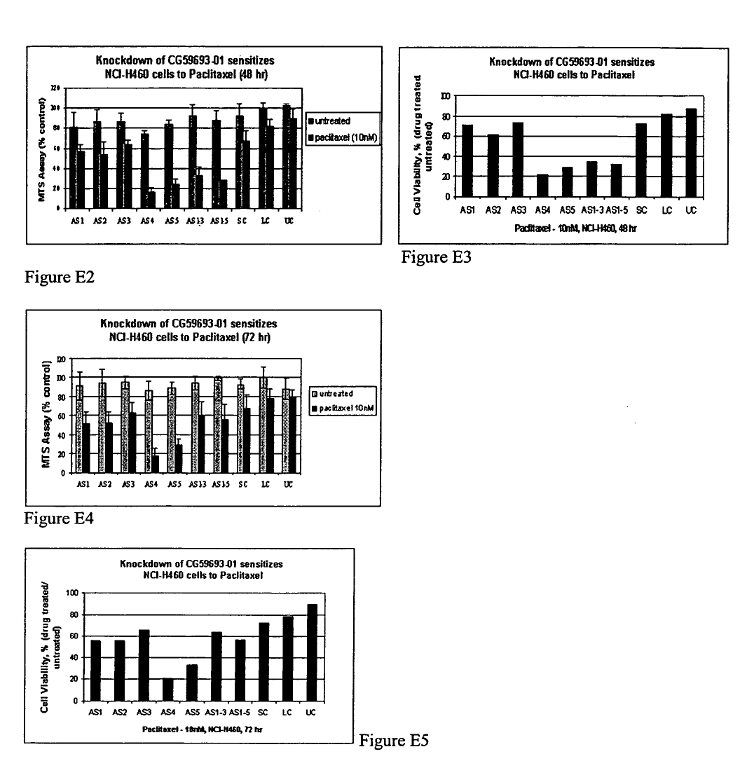

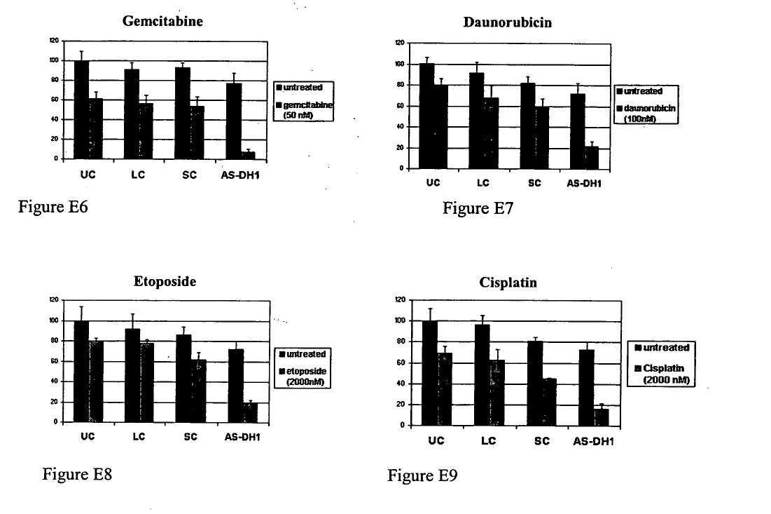

### RELATED APPLICATIONS

This application claims priority to U.S. Ser. No. 60/385,615, filed Jun. 4, 2002; U.S. Ser. No. 60/402,268, filed Aug. 9, 2002; U.S. Ser. No. 60/387,606, filed Jun. 11, 2002; U.S. Ser. No. 60/386,357, filed Jun. 6, 2002; U.S. Ser. No. 60/385,755, filed Jun. 4, 2002; U.S. Ser. No. 60/386,355, filed Jun. 6, 2002; U.S. Ser. No. 60/385,490, filed Jun. 4, 2002; U.S. Ser. No. 60/420,718, filed Oct. 23, 2002; U.S. Ser. No. 60/386,447, filed Jun. 6, 2002; U.S. Ser. No. 60/386,465, filed Jun. 6, 2002; U.S. Ser. No. 60/420,627, filed Oct. 23, 2002; U.S. Ser. No. 60/386,459, filed Jun. 6, 2002; U.S. Ser. No. 60/410,505, filed Sep. 13, 2002; U.S. Ser. No. 60/420,852, filed Oct. 24, 2002; U.S. Ser. No. 60/386,796, filed Jun. 7, 2002; U.S. Ser. No. 60/387,078, filed Jun. 7, 2002; U.S. Ser. No. 60/387,083, filed Jun. 7, 2002; U.S. Ser. No. 60/387,081, filed Jun. 7, 2002; U.S. Ser. No. 60/386,041, filed Jun. 5, 2002; U.S. Ser. No. 60/386,701, filed Jun. 7, 2002; U.S. Ser. No. 60/387,610, filed Jun. 11, 2002; U.S. Ser. No. 60/387,540, filed Jun. 10, 2002; U.S. Ser. No. 60/387,429, filed Jun. 10, 2002; U.S. Ser. No. 60/410,085, filed Sep. 12, 2002; U.S. Ser. No. 60/389,120, filed Jun. 14, 2002; U.S. Ser. No. 60/386,931, filed Jun. 7, 2002; U.S. Ser. No. 60/387,866, filed Jun. 10, 2002; U.S. Ser. No. 60/387,859, filed Jun. 11, 2002; U.S. Ser. No. 60/387,659, filed Jun. 11, 2002; U.S. Ser. No. 60/387,934, filed Jun. 12, 2002; U.S. Ser. No. 60/387,696, filed Jun. 11, 2002; U.S. Ser. No. 60/390,006, filed Jun. 19, 2002; U.S. Ser. No. 60/389,604, filed Jun. 18, 2002; U.S. Ser. No. 60/387,668, filed Jun. 11, 2002; U.S. Ser. No. 60/386,864, filed Jun. 6, 2002; U.S. Ser. No. 60/401,628, filed Aug. 6, 2002; U.S. Ser. No. 60/406,182, filed Aug. 26, 2002; U.S. Ser. No. 60/412,955, filed Sep. 23, 2002; U.S. Ser. No. 60/415,195, filed Sep. 30, 2002; U.S. Ser. No. 60/422,750, filed Oct. 31, 2002; U.S. Ser. No. 60/390,144, filed Jun. 19, 2002; U.S. Ser. No. 60/388,022, filed Jun. 12, 2002; U.S. Ser. No. 60/402,822, filed Aug. 12, 2002; U.S. Ser. No. 60/388,096, filed Jun. 12, 2002; U.S. Ser. No. 60/389,123, filed Jun. 13, 2002; U.S. Ser. No. 60/390,209, filed Jun. 19, 2002; U.S. Ser. No. 60/388,479, filed Jun. 12, 2002; U.S. Ser. No. 60/403,458, filed Aug. 13, 2002; U.S. Ser. No. 60/389,884, filed Jun. 18, 2002; U.S. Ser. No. 60/389,146, filed Jun. 14, 2002; U.S. Ser. No. 60/387,960, filed Jun. 12, 2002; U.S. Ser. No. 60/388,432, filed Jun. 12, 2002; U.S. Ser. No. 60/403,617, filed Aug. 15, 2002; U.S. Ser. No. 60/423,095, filed Nov. 1, 2002; U.S. Ser. No. 60/423,748, filed Nov. 5, 2002; U.S. Ser. No. 60/391,726, filed Jun. 25, 2002; U.S. Ser. No. 60/403,732, filed Aug. 15, 2002; and U.S. Ser. No. 60/389,742, filed Jun. 17, 2002, and is a continuation in part of U.S. Ser. No. 09/669,360, filed on Sep. 26, 2000, which claims priority to U.S. Ser. No. 60/156,217, filed on Sep. 27, 1999; a continuation in part of U.S. Ser. No. 09/795,271, filed on Feb. 27, 2001, which claims priority to U.S. Ser. No. 60/264,849, filed on Jan. 29, 2001; a continuation in part of U.S. Ser. No. 09/800,198, filed on Mar. 5, 2001, which claims priority to U.S. Ser. No. 60/196,018, filed on Apr. 7, 2000; a continuation in part of U.S. Ser. No. 09/844,861, filed on Apr. 27, 2001, which claims the benefit of U.S. Ser. No. 60/199,947, filed on Apr. 27, 2000; a continuation in part of U.S. Ser. No. 849,861, filed on May 4, 2001, which claims priority to U.S. Ser. No. 60/201,951, filed on May 5, 2000; a continuation in part of U.S. Ser. No. 10/038,854, filed on Dec. 31, 2001, which claims priority to U.S. Ser. No. 60/322,699, filed on Sep. 17, 2001 and U.S. Ser. No. 60/286,683, filed on Apr. 25, 2001; a continuation in part of U.S. Ser. No. 10/051,874, filed on Jan. 16, 2002, which claims priority to U.S. Ser. No. 60/262,454, filed on Jan. 18, 2001, U.S. Ser. No. 60/276,777, filed on Mar. 16, 2001, and U.S. Ser. No. 60/291,672, filed on May 17, 2001; a continuation in part of U.S. Ser. No. 10/092,900, filed on Mar. 7, 2002, which claims priority to U.S. Ser. No. 60/275,235, filed on Mar. 12, 2001; a continuation in part of U.S. Ser. No. 10/136,071, filed on May 1, 2002, which claims priority to U.S. Ser. No. 60/293,589 and U.S. Ser. No. 60/298,484, filed on Jun. 15, 2001; a continuation in part of U.S. Ser. No. 10/136,826, filed on May 1, 2002, which claims priority to U.S. Ser. No. 60/228,8063, filed on May 2, 2001; a continuation in part of U.S. Ser. No. 10/160,619, filed on Jun. 3, 2002, which claims priority to U.S. Ser. No. 60/295,661, filed on Jun. 4, 2001; a continuation in part of U.S. Ser. No. 09/783,429, filed on Feb. 14, 2001; a continuation in part of U.S. Ser. No. 09/800,321, filed on Mar. 5, 2001; a continuation in part of U.S. Ser. No. 09/832,522, filed on Apr. 11, 2001; a continuation in part of U.S. Ser. No. 09/995,514, filed on Nov. 28, 2001; a continuation in part of U.S. Ser. No. 10/023,634, filed on Dec. 17, 2001; a continuation in part of U.S. Ser. No. 10/028,248, filed on Dec. 19, 2001; a continuation in part of U.S. Ser. No. 10/038,854, filed on Dec. 31, 2001; a continuation in part of U.S. Ser. No. 10/055,877, filed on Jan. 22, 2002; a continuation in part of U.S. Ser. No. 10/092,900, filed on Mar. 7, 2002; a continuation in part of U.S. Ser. No. 10/114,153, filed on Apr. 2, 2002; a continuation in part of U.S. Ser. No. 10/115,479, filed on Apr. 2, 2002; and a continuation in part of U.S. Ser. No. 10/136,826, filed on May 1, 2002, each of which is incorporated by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors: In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 566, or polypeptide sequences, which represents the group consisting of SEQ ID NO: 2n, wherein n is an integer between 1 and 566.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 566, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. D1: Alignment of CG55806-04 (SEQ ID NO:748), CG55806-02 (SEQ ID NO:752), and 1PFX (SEQ ID NO: 1476).

FIG. D2: Structure of porcine factor IXa (1PFX).

FIG. E1: Data showing effect on cell growth by knockdown of CG59693-01.

FIG. E2: Data showing effect on cell growth by knockdown of CG59693-01 with subsequent treatment with Paclitaxel (48 hr).

FIG. E3: Data showing effect on cell viability by knockdown of CG59693-01 with subsequent treatment with Paclitaxel (48 hr).

FIG. E4: Data showing effect on cell growth by knockdown of CG59693-01 with subsequent treatment with Paclitaxel (72 hr).

FIG. E5: Data showing effect on cell viability by knockdown of CG59693-01 with subsequent treatment with Paclitaxel (72 hr).

FIG. E6: Data showing effect on cell growth by knockdown of CG59693-01 by AS4 antisense oligonucleotide followed by subsequent treatment with Gemcitabine.

FIG. E7: Data showing effect on cell growth by knockdown of CG59693-01 by AS4 antisense oligonucleotide followed by subsequent treatment with Daunorubicin.

FIG. E8: Data showing effect on cell growth by knockdown of CG59693-01 by AS4 antisense oligonucleotide followed by subsequent treatment with Etoposide.

FIG. E9: Data showing effect on cell growth by knockdown of CG59693-01 by AS4 antisense oligonucleotide followed by subsequent treatment with Cisplatin.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ | |
|---|---|---|---|---|
| | | NO | ID NO | |
| NOVX | Internal | (nucleic | (amino | |
| Assignment | Identification | acid) | acid) | Homology |
| NOV1a | CG101340-01 | 1 | 2 | Putative G protein-coupled receptor |
| | | | | 92 (Putative G-protein coupled |
| | | | | receptor) - Homo sapiens |
| NOV1b | SNP13382465 | 3 | 4 | Putative G protein-coupled receptor |
| | | | | 92 (Putative G-protein coupled |
| | | | | receptor) - Homo sapiens |
| NOV2a | CG101396-01 | 5 | 6 | Glutamate receptor delta-1 subunit |
| | | | | precursor - Rattus norvegicus |
| NOV2b | 267253224 | 7 | 8 | Glutamate receptor delta-1 subunit |
| | | | | precursor - Rattus norvegicus |
| NOV2c | 315490179 | 9 | 10 | Glutamate receptor delta-1 subunit |
| | | | | precursor - Rattus norvegicus |
| NOV2d | CG101396-02 | 11 | 12 | Glutamate receptor delta-1 subunit |
| | | | | precursor - Rattus norvegicus |
| NOV2e | SNP13379211 | 13 | 14 | Glutamate receptor delta-1 subunit |
| | | | | precursor - Rattus norvegicus |
| NOV3a | CG102348-01 | 15 | 16 | Complement Clr-like proteinase - |
| | | | | Homo sapiens |
| NOV3b | 199842645 | 17 | 18 | Complement Clr-like proteinase - |
| | | | | Homo sapiens |
| NOV3c | 198306343 | 19 | 20 | Complement Clr-like proteinase - |
| | | | | Homo sapiens |
| NOV3d | 199842665 | 21 | 22 | Complement Clr-like proteinase - |
| | | | | Homo sapiens |
| NOV3e | 199842661 | 23 | 24 | Complement Clr-like proteinase - |
| | | | | Homo sapiens |
| NOV3f | 199597024 | 25 | 26 | Complement Clr-like proteinase - |
| | | | | Homo sapiens |
| NOV3g | 199842653 | 27 | 28 | Complement Clr-like proteinase - |
| | | | | Homo sapiens |
| NOV3h | 199652830 | 29 | 30 | Complement Clr-like proteinase - |
| | | | | Homo sapiens |
| NOV3i | 199652835 | 31 | 32 | Complement Clr-like proteinase - |
| | | | | Homo sapiens |
| NOV3j | 198306308 | 33 | 34 | Complement Clr-like proteinase - |
| | | | | Homo sapiens |
| NOV3k | CG102348-02 | 35 | 36 | Complement Clr-like proteinase - |
| | | | | Homo sapiens |
| NOV3l | CG102348-03 | 37 | 38 | Complement Clr-like proteinase - |
| | | | | Homo sapiens |
| NOV3m | CG102348-04 | 39 | 40 | Complement Clr-like proteinase - |
| | | | | Homo sapiens |
| NOV3n | CG102348-05 | 41 | 42 | Complement Clr-like proteinase - |
| | | | | Homo sapiens |
| NOV3o | CG102348-06 | 43 | 44 | Complement Clr-like proteinase - |
| | | | | Homo sapiens |
| NOV3p | SNP13376570 | 45 | 46 | Complement Clr-like proteinase - |
| | | | | Homo sapiens |
| NOV3q | SNP13376568 | 47 | 48 | Complement Clr-like proteinase - |
| | | | | Homo sapiens |
| NOV3r | SNP13382463 | 49 | 50 | Complement Clr-like proteinase - |
| | | | | Homo sapiens |
| NOV3s | SNP13374245 | 51 | 52 | Complement Clr-like proteinase - |
| | | | | Homo sapiens |
| NOV4a | CG125860-02 | 53 | 54 | Transmembrane protease, serine 5 |
| | | | | (EC 3.4.21.—) (Spinesin) - Homo |
| | | | | sapiens |
| NOV4b | CG125860-01 | 55 | 56 | Transmembrane protease, serine 5 |
| | | | | (EC 3.4.21.—) (Spinesin) - Homo |
| | | | | sapiens |
| NOV4c | SNP13376012 | 57 | 58 | Transmembrane protease, serine 5 |
| | | | | (EC 3.4.21.—) (Spinesin) - Homo |
| | | | | sapiens |
| NOV4d | SNP13376014 | 59 | 60 | Transmembrane protease, serine 5 |
| | | | | (EC 3.4.21.—) (Spinesin) - Homo |
| | | | | sapiens |
| NOV4e | SNP13376015 | 61 | 62 | Transmembrane protease, serine 5 |
| | | | | (EC 3.4.21.—) (Spinesin) - Homo |
| | | | | sapiens |
| NOV4f | SNP13376016 | 63 | 64 | Transmembrane protease, serine 5 |
| | | | | (EC 3.4.21.—) (Spinesin) - Homo |
| | | | | sapiens |
| NOV4g | SNP13376017 | 65 | 66 | Transmembrane protease, serine 5 |
| | | | | (EC 3.4.21.—) (Spinesin) - Homo |
| | | | | sapiens |
| NOV4h | SNP13376011 | 67 | 68 | Transmembrane protease, serine 5 |
| | | | | (EC 3.4.21.—) (Spinesin) - Homo |
| | | | | sapiens |
| NOV4i | SNP13376018 | 69 | 70 | Transmembrane protease, serine 5 |
| | | | | (EC 3.4.21.—) (Spinesin) - Homo |
| | | | | sapiens |
| NOV4j | SNP13382467 | 71 | 72 | Transmembrane protease, serine 5 |
| | | | | (EC 3.4.21.—) (Spinesin) - Homo |
| | | | | sapiens |
| NOV4k | SNP13382466 | 73 | 74 | Transmembrane protease, serine 5 |
| | | | | (EC 3.4.21.—) (Spinesin) - Homo |
| | | | | sapiens |
| NOV5a | CG155759-02 | 75 | 76 | Seven transmembrane helix receptor - |
| | | | | Homo sapiens |
| NOV5b | CG155759-01 | 77 | 78 | Seven transmembrane helix receptor - |
| | | | | Homo sapiens |
| NOV6a | CG187667-01 | 79 | 80 | LOC133308 protein - Homo sapiens |
| NOV6b | CG187667-02 | 81 | 82 | LOC133308 protein - Homo sapiens |
| NOV7a | CG187676-01 | 83 | 84 | Sentrin-specific protease 5 (EC |
| | | | | 3.4.22.—) (Sentrin/SUMO-specific |
| | | | | protease SENP5) (Protease FKSG45) - |
| | | | | Homo sapiens |
| NOV8a | CG50235-04 | 85 | 86 | Tolloid-like 2 protein - Homo sapiens |
| NOV8b | CG50235-01 | 87 | 88 | Tolloid-like 2 protein - Homo sapiens |
| NOV8c | CG50235-02 | 89 | 90 | Tolloid-like 2 protein - Homo sapiens |
| NOV8d | CG50235-03 | 91 | 92 | Tolloid-like 2 protein - Homo sapiens |
| NOV8e | SNP13377383 | 93 | 94 | Tolloid-like 2 protein - Homo sapiens |
| NOV8f | SNP13377384 | 95 | 96 | Tolloid-like 2 protein - Homo sapiens |
| NOV8g | SNP13377385 | 97 | 98 | Tolloid-like 2 protein - Homo sapiens |
| NOV8h | SNP13377386 | 99 | 100 | Tolloid-like 2 protein - Homo sapiens |
| NOV8i | SNP13377387 | 101 | 102 | Tolloid-like 2 protein - Homo sapiens |
| NOV9a | CG50249-01 | 103 | 104 | Voltage gated potassium channel |
| | | | | Kv3.2b (Potassium voltage-gated |
| | | | | potassium channel subfamily C |
| | | | | member 2) - Homo sapiens |
| NOV9b | 207885588 | 105 | 106 | Voltage gated potassium channel |
| | | | | Kv3.2b (Potassium voltage-gated |
| | | | | potassium channel subfamily C |
| | | | | member 2) - Homo sapiens |
| NOV9c | CG50249-02 | 107 | 108 | Voltage gated potassium channel |
| | | | | Kv3.2b (Potassium voltage-gated |
| | | | | potassium channel subfamily C |
| | | | | member 2) - Homo sapiens |
| NOV9d | CG50249-03 | 109 | 110 | Voltage gated potassium channel |
| | | | | Kv3.2b (Potassium voltage-gated |
| | | | | potassium channel subfamily C |
| | | | | member 2) - Homo sapiens |
| NOV9e | CG50249-04 | 111 | 112 | Voltage gated potassium channel |
| | | | | Kv3.2b (Potassium voltage-gated |
| | | | | potassium channel subfamily C |
| | | | | member 2) - Homo sapiens |
| NOV10a | CG50307-03 | 113 | 114 | Steroid dehydrogenase-like protein |
| NOV10b | 275624102 | 115 | 116 | Steroid dehydrogenase-like protein |
| NOV10c | CG50307-01 | 117 | 118 | Steroid dehydrogenase-like protein |
| NOV10d | CG50307-02 | 119 | 120 | Steroid dehydrogenase-like protein |
| NOV10e | SNP13375811 | 121 | 122 | Steroid dehydrogenase-like protein |
| NOV11a | CG50315-01 | 123 | 124 | Seven transmembrane helix receptor - |
| | | | | Homo sapiens |
| NOV11b | 207580272 | 125 | 126 | Seven transmembrane helix receptor - |
| | | | | Homo sapiens |
| NOV11c | 314411778 | 127 | 128 | Seven transmembrane helix receptor - |
| | | | | Homo sapiens |
| NOV11d | CG50315-02 | 129 | 130 | Seven transmembrane helix receptor - |
| | | | | Homo sapiens |
| NOV11e | CG50315-03 | 131 | 132 | Seven transmembrane helix receptor - |
| | | | | Homo sapiens |
| NOV12a | CG50341-01 | 133 | 134 | Seven transmembrane helix receptor - |
| | | | | Homo sapiens |
| NOV12b | 169475616 | 135 | 136 | Seven transmembrane helix receptor - |
| | | | | Homo sapiens |
| NOV12c | CG50341-02 | 137 | 138 | Seven transmembrane helix receptor - |
| | | | | Homo sapiens |
| NOV12d | CG50341-03 | 139 | 140 | Seven transmembrane helix receptor - |
| | | | | Homo sapiens |
| NOV12e | CG50341-04 | 141 | 142 | Seven transmembrane helix receptor - |
| | | | | Homo sapiens |
| NOV13a | CG50365-01 | 143 | 144 | Carbonic anhydrase XIII (EC 4.2.1.1) |
| | | | | (Carbonate dehydratase XIII) (CA- |
| | | | | XIII) - Homo sapiens |
| NOV13b | 278019595 | 145 | 146 | Carbonic anhydrase XIII (EC 4.2.1.1) |
| | | | | (Carbonate dehydratase XIII) (CA- |
| | | | | XIII) - Homo sapiens |
| NOV13c | CG50365-02 | 147 | 148 | Carbonic anhydrase XIII (EC 4.2.1.1) |
| | | | | (Carbonate dehydratase XIII) (CA- |
| | | | | XIII) - Homo sapiens |
| NOV14a | CG50367-01 | 149 | 150 | ADAM 33 precursor (EC 3.4.24.—) (A |
| | | | | disintegrin and metalloproteinase |
| | | | | domain 33) - Homo sapiens |
| NOV14b | CG50367-05 | 151 | 152 | ADAM 33 precursor (EC 3.4.24.—) (A |
| | | | | disintegrin and metalloproteinase |
| | | | | domain 33) - Homo sapiens |
| NOV14c | CG50367-06 | 153 | 154 | ADAM 33 precursor (EC 3.4.24.—) (A |
| | | | | disintegrin and metalloproteinase |
| | | | | domain 33) - Homo sapiens |
| NOV14d | CG50367-07 | 155 | 156 | ADAM 33 precursor (EC 3.4.24.—) (A |
| | | | | disintegrin and metalloproteinase |
| | | | | domain 33) - Homo sapiens |
| NOV14e | 249356906 | 157 | 158 | ADAM 33 precursor (EC 3.4.24.—) (A |
| | | | | disintegrin and metalloproteinase |
| | | | | domain 33) - Homo sapiens |
| NOV14f | CG50367-02 | 159 | 160 | ADAM 33 precursor (EC 3.4.24.—) (A |
| | | | | disintegrin and metalloproteinase |
| | | | | domain 33) - Homo sapiens |
| NOV14g | CG50367-03 | 161 | 162 | ADAM 33 precursor (EC 3.4.24.—) (A |
| | | | | disintegrin and metalloproteinase |
| | | | | domain 33) - Homo sapiens |
| NOV14h | CG50367-04 | 163 | 164 | ADAM 33 precursor (EC 3.4.24.—) (A |
| | | | | disintegrin and metalloproteinase |
| | | | | domain 33) - Homo sapiens |
| NOV15a | CG50718-02 | 165 | 166 | Glomerular mesangial cell receptor |
| | | | | protein-tyrosine phosphatase |
| | | | | precursor - Rattus norvegicus |
| NOV15b | CG50718-06 | 167 | 168 | Glomerular mesangial cell receptor |
| | | | | protein-tyrosine phosphatase |
| | | | | precursor - Rattus norvegicus |
| NOV15c | 258979883 | 169 | 170 | Glomerular mesangial cell receptor |
| | | | | protein-tyrosine phosphatase |
| | | | | precursor - Rattus norvegicus |
| NOV15d | CG50718-01 | 171 | 172 | Glomerular mesangial cell receptor |
| | | | | protein-tyrosine phosphatase |
| | | | | precursor - Rattus norvegicus |
| NOV15e | CG50718-03 | 173 | 174 | Glomerular mesangial cell receptor |
| | | | | protein-tyrosine phosphatase |
| | | | | precursor - Rattus norvegicus |
| NOV15f | CG50718-04 | 175 | 176 | Glomerular mesangial cell receptor |
| | | | | protein-tyrosine phosphatase |
| | | | | precursor - Rattus norvegicus |
| NOV15g | CG50718-05 | 177 | 178 | Glomerular mesangial cell receptor |
| | | | | protein-tyrosine phosphatase |
| | | | | precursor - Rattus norvegicus |
| NOV16a | CG50934-03 | 179 | 180 | Mast cell protease-11 - Mus musculus |
| NOV16b | CG50934-01 | 181 | 182 | Mast cell protease-11 - Mus musculus |
| NOV16c | CG50934-02 | 183 | 184 | Mast cell protease-11 - Mus musculus |
| NOV16d | SNP13381559 | 185 | 186 | Mast cell protease-11 - Mus musculus |
| NOV16e | SNP13381558 | 187 | 188 | Mast cell protease-11 - Mus musculus |
| NOV16f | SNP13376399 | 189 | 190 | Mast cell protease-11 - Mus musculus |
| NOV16g | SNP13378301 | 191 | 192 | Mast cell protease-11 - Mus musculus |
| NOV16h | SNP13378300 | 193 | 194 | Mast cell protease-11 - Mus musculus |
| NOV17a | CG51213-04 | 195 | 196 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17b | CG51213-01 | 197 | 198 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17c | 306345264 | 199 | 200 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17d | CG51213-02 | 201 | 202 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17e | CG51213-03 | 203 | 204 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17f | CG51213-05 | 205 | 206 | ADAMTS-10 precursor (EG 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17g | CG51213-06 | 207 | 208 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV17h | CG51213-07 | 209 | 210 | ADAMTS-10 precursor (EC 3.4.24.—) |
| | | | | (A disintegrin and metalloproteinase |
| | | | | with thrombospondin motifs 10) |
| | | | | (ADAM-TS 10) (ADAM-TS10) - |
| | | | | Homo sapiens (Human), 1077 aa |
| NOV18a | CG51448-05 | 211 | 212 | Human cardiac myosin light chain |
| | | | | kinase (cMLCK) mutant, G89D |
| NOV18b | CG51448-01 | 213 | 214 | Human cardiac myosin light chain |
| | | | | kinase (cMLCK) mutant, G89D |
| NOV18c | 274051198 | 215 | 216 | Human cardiac myosin light chain |
| | | | | kinase (cMLCK) mutant, G89D |
| NOV18d | 274051170 | 217 | 218 | Human cardiac myosin light chain |
| | | | | kinase (cMLCK) mutant, G89D |
| NOV18e | CG51448-02 | 219 | 220 | Human cardiac myosin light chain |
| | | | | kinase (cMLCK) mutant, G89D |
| NOV18f | CG51448-03 | 221 | 222 | Human cardiac myosin light chain |
| | | | | kinase (cMLCK) mutant, G89D |
| NOV18g | CG51448-04 | 223 | 224 | Human cardiac myosin light chain |
| | | | | kinase (cMLCK) mutant, G89D |
| NOV18h | SNP13375535 | 225 | 226 | Human cardiac myosin light chain |
| | | | | kinase (cMLCK) mutant, G89D |
| NOV18i | SNP13375536 | 227 | 228 | Human cardiac myosin light chain |
| | | | | kinase (cMLCK) mutant, G89D |
| NOV18j | SNP13375537 | 229 | 230 | Human cardiac myosin light chain |
| | | | | kinase (cMLCK) mutant, G89D |
| NOV18k | SNP13375538 | 231 | 232 | Human cardiac myosin light chain |
| | | | | kinase (cMLCK) mutant, G89D |
| NOV18l | SNP13375539 | 233 | 234 | Human cardiac myosin light chain |
| | | | | kinase (cMLCK) mutant, G89D |
| NOV18m | SNP13375540 | 235 | 236 | Human cardiac myosin light chain |
| | | | | kinase (cMLCK) mutant, G89D |
| NOV18n | SNP13375541 | 237 | 238 | Human cardiac myosin light chain |
| | | | | kinase (cMLCK) mutant, G89D |
| NOV18o | SNP13375542 | 239 | 240 | Human cardiac myosin light chain |
| | | | | kinase (cMLCK) mutant, G89D |
| NOV18p | SNP13375543 | 241 | 242 | Human cardiac myosin light chain |
| | | | | kinase (cMLCK) mutant, G89D |
| NOV19a | CG51752-02 | 243 | 244 | Human FCTR6b polypeptide |
| | | | | sequence |
| NOV19b | CG51752-03 | 245 | 246 | Human FCTR6b polypeptide |
| | | | | sequence |
| NOV19c | 175069825 | 247 | 248 | Human FCTR6b polypeptide |
| | | | | sequence |
| NOV19d | 175069842 | 249 | 250 | Human FCTR6b polypeptide |
| | | | | sequence |
| NOV19e | 258076315 | 251 | 252 | Human FCTR6b polypeptide |
| | | | | sequence |
| NOV19f | 258076366 | 253 | 254 | Human FCTR6b polypeptide |
| | | | | sequence |
| NOV19g | CG51752-04 | 255 | 256 | Human FCTR6b polypeptide |
| | | | | sequence |
| NOV19h | 191887409 | 257 | 258 | Human FCTR6b polypeptide |
| | | | | sequence |
| NOV19i | CG51752-01 | 259 | 260 | Human FCTR6b polypeptide |
| | | | | sequence |
| NOV19j | CG51752-05 | 261 | 262 | Human FCTR6b polypeptide |
| | | | | sequence |
| NOV19k | CG51752-06 | 263 | 264 | Human FCTR6b polypeptide |
| | | | | sequence |
| NOV19l | CG51752-07 | 265 | 266 | Human FCTR6b polypeptide |
| | | | | sequence |
| NOV19m | SNP13374584 | 267 | 268 | Human FCTR6b polypeptide |
| | | | | sequence |
| NOV19n | SNP13374585 | 269 | 270 | Human FCTR6b polypeptide |
| | | | | sequence |
| NOV20a | CG51914-02 | 271 | 272 | Human kinase polypeptide (PKIN-16) |
| NOV20b | CG51914-01 | 273 | 274 | Human kinase polypeptide (PKIN-16) |
| NOV21a | CG51965-01 | 275 | 276 | Cadherin EGF LAG seven-pass G- |
| | | | | type receptor 1 precursor (Flamingo |
| | | | | homolog 2) (hFmi2) - Homo sapiens |
| NOV21b | 258076370 | 277 | 278 | Cadherin EGF LAG seven-pass G- |
| | | | | type receptor 1 precursor (Flamingo |
| | | | | homolog 2) (hFmi2) - Homo sapiens |
| NOV21c | 317619862 | 279 | 280 | Cadherin EGF LAG seven-pass G- |
| | | | | type receptor 1 precursor (Flamingo |
| | | | | homolog 2) (hFmi2) - Homo sapiens |
| NOV21d | 317460050 | 281 | 282 | Cadherin EGF LAG seven-pass G- |
| | | | | type receptor 1 precursor (Flamingo |
| | | | | homolog 2) (hFmi2) - Homo sapiens |
| NOV21e | SNP13382483 | 283 | 284 | Cadherin EGF LAG seven-pass G- |
| | | | | type receptor 1 precursor (Flamingo |
| | | | | homolog 2) (hFmi2) - Homo sapiens |
| NOV21f | SNP13382484 | 285 | 286 | Cadherin EGF LAG seven-pass G- |
| | | | | type receptor 1 precursor (Flamingo |
| | | | | homolog 2) (hFmi2) - Homo sapiens |
| NOV21g | SNP13382485 | 287 | 288 | Cadherin EGF LAG seven-pass G- |
| | | | | type receptor 1 precursor (Flamingo |
| | | | | homolog 2) (hFmi2) - Homo sapiens |
| NOV22a | CG51983-05 | 289 | 290 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22b | CG51983-01 | 291 | 292 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22c | CG51983-02 | 293 | 294 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22d | CG51983-03 | 295 | 296 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22e | CG51983-04 | 297 | 298 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22f | CG51983-06 | 299 | 300 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22g | SNP13376585 | 301 | 302 | ADAM 7 precursor (A disintegrin and |
| | | | | metalloproteinase domain 7) (Sperm |
| | | | | maturation-related glycoprotein GP- |
| | | | | 83) - Homo sapiens |
| NOV22h | SNP13376586 | 303 | 304 | ADAM 7 precursor (A disintegrin and |
| | | | | metalloproteinase domain 7) (Sperm |
| | | | | maturation-related glycoprotein GP- |
| | | | | 83) - Homo sapiens |
| NOV23a | CG53390-02 | 305 | 306 | Seven transmembrane helix receptor - |
| | | | | Homo sapiens |
| NOV23b | CG53390-01 | 307 | 308 | Seven transmembrane helix receptor - |
| | | | | Homo sapiens |
| NOV23c | CG53390-03 | 309 | 310 | Seven transmembrane helix receptor - |
| | | | | Homo sapiens |
| NOV24a | CG53482-01 | 311 | 312 | Human odorant receptor (OR) |
| NOV24b | CG53482-02 | 313 | 314 | Human odorant receptor (OR) |
| NOV24c | CG53482-03 | 315 | 316 | Human odorant receptor (OR) |
| NOV24d | SNP13373787 | 317 | 318 | Human odorant receptor (OR) |
| NOV24e | SNP13373786 | 319 | 320 | Human odorant receptor (OR) |
| NOV24f | SNP13373785 | 321 | 322 | Human odorant receptor (OR) |
| NOV25a | CG53530-03 | 323 | 324 | Seven transmembrane helix receptor - |
| | | | | Homo sapiens |
| NOV25b | CG53530-01 | 325 | 326 | Seven transmembrane helix receptor - |
| | | | | Homo sapiens |
| NOV25c | CG53530-02 | 327 | 328 | Seven transmembrane helix receptor - |
| | | | | Homo sapiens |
| NOV26a | CG53563-03 | 329 | 330 | Seven transmembrane helix receptor - |
| | | | | Homo sapiens |
| NOV26b | CG53563-01 | 331 | 332 | Seven transmembrane helix receptor - |
| | | | | Homo sapiens |
| NOV26c | CG53563-02 | 333 | 334 | Seven transmembrane helix receptor - |
| | | | | Homo sapiens |
| NOV27a | CG53719-02 | 335 | 336 | Olfactory receptor MOR106-5 - Mus |
| | | | | musculus |
| NOV27b | CG53719-01 | 337 | 338 | Olfactory receptor MOR106-5 - Mus |
| | | | | musculus |
| NOV28a | CG53746-04 | 339 | 340 | Olfactory receptor 5P3 (Olfactory |
| | | | | receptor-like protein JCG1) - Homo |
| | | | | sapiens |
| NOV28b | CG53746-01 | 341 | 342 | Olfactory receptor 5P3 (Olfactory |
| | | | | receptor-like protein JCG1) - Homo |
| | | | | sapiens |
| NOV28c | CG53746-02 | 343 | 344 | Olfactory receptor 5P3 (Olfactory |
| | | | | receptor-like protein JCG1) - Homo |
| | | | | sapiens |
| NOV28d | CG53746-03 | 345 | 346 | Olfactory receptor 5P3 (Olfactory |
| | | | | receptor-like protein JCG1) - Homo |
| | | | | sapiens |
| NOV28e | SNP13373967 | 347 | 348 | Olfactory receptor 5P3 (Olfactory |
| | | | | receptor-like protein JCG1) - Homo |
| | | | | sapiens |
| NOV28f | SNP13373968 | 349 | 350 | Olfactory receptor 5P3 (Olfactory |
| | | | | receptor-like protein JCG1) - Homo |
| | | | | sapiens |
| NOV28g | SNP13382432 | 351 | 352 | Olfactory receptor 5P3 (Olfactory |
| | | | | receptor-like protein JCG1) - Homo |
| | | | | sapiens |
| NOV28h | SNP13373969 | 353 | 354 | Olfactory receptor 5P3 (Olfactory |
| | | | | receptor-like protein JCG1) - Homo |
| | | | | sapiens |
| NOV28i | SNP13373970 | 355 | 356 | Olfactory receptor 5P3 (Olfactory |
| | | | | receptor-like protein JCG1) - Homo |
| | | | | sapiens |
| NOV28j | SNP13373971 | 357 | 358 | Olfactory receptor 5P3 (Olfactory |
| | | | | receptor-like protein JCG1) - Homo |
| | | | | sapiens |
| NOV28k | SNP13373972 | 359 | 360 | Olfactory receptor 5P3 (Olfactory |
| | | | | receptor-like protein JCG1) - Homo |
| | | | | sapiens |
| NOV28l | SNP13382433 | 361 | 362 | Olfactory receptor 5P3 (Olfactory |
| | | | | receptor-like protein JCG1) - Homo |
| | | | | sapiens |
| NOV29a | CG53767-02 | 363 | 364 | Seven transmembrane helix receptor - |
| | | | | Homo sapiens |
| NOV29b | CG53767-01 | 365 | 366 | Seven transmembrane helix receptor - |
| | | | | Homo sapiens |
| NOV29c | SNP13382437 | 367 | 368 | Seven transmembrane helix receptor - |
| | | | | Homo sapiens |
| NOV29d | SNP13382436 | 369 | 370 | Seven transmembrane helix receptor - |
| | | | | Homo sapiens |
| NOV30a | CG53776-02 | 371 | 372 | Olfactory receptor 5P2 (Olfactory |
| | | | | receptor-like protein JCG3) - Homo |
| | | | | sapiens |
| NOV30b | CG53776-03 | 373 | 374 | Olfactory receptor 5P2 (Olfactory |
| | | | | receptor-like protein JCG3) - Homo |
| | | | | sapiens |
| NOV30c | CG53776-01 | 375 | 376 | Olfactory receptor 5P2 (Olfactory |
| | | | | receptor-like protein JCG3) - Homo |
| | | | | sapiens |
| NOV31a | CG53803-02 | 377 | 378 | Sequence 3 from Patent WO0159113 - |
| | | | | Homo sapiens |
| NOV31b | CG53803-01 | 379 | 380 | Sequence 3 from Patent WO0159113 - |
| | | | | Homo sapiens |
| NOV32a | CG53989-03 | 381 | 382 | Mast cell protease-11 - Mus musculus |
| NOV32b | CG53989-04 | 383 | 384 | Mast cell protease-11 - Mus musculus |
| NOV32c | 306076095 | 385 | 386 | Mast cell protease-11 - Mus musculus |
| NOV32d | CG53989-01 | 387 | 388 | Mast cell protease-11 - Mus musculus |
| NOV32e | CG53989-02 | 389 | 390 | Mast cell protease-11 - Mus musculus |
| NOV32f | CG53989-05 | 391 | 392 | Mast cell protease-11 - Mus musculus |
| NOV32g | CG53989-06 | 393 | 394 | Mast cell protease-11 - Mus musculus |
| NOV32h | CG53989-07 | 395 | 396 | Mast cell protease-11 - Mus musculus |
| NOV32i | CG53989-08 | 397 | 398 | Mast cell protease-11 - Mus musculus |
| NOV32j | CG53989-09 | 399 | 400 | Mast cell protease-11 - Mus musculus |
| NOV32k | CG53989-10 | 401 | 402 | Mast cell protease-11 - Mus musculus |
| NOV32l | CG53989-11 | 403 | 404 | Mast cell protease-11 - Mus musculus |
| NOV32m | CG53989-12 | 405 | 406 | Mast cell protease-11 - Mus musculus |
| NOV32n | CG53989-13 | 407 | 408 | Mast cell protease-11 - Mus musculus |
| NOV33a | CG54203-02 | 409 | 410 | Olfactory receptor 2S2 - Homo |
| | | | | sapiens |
| NOV33b | CG54203-01 | 411 | 412 | Olfactory receptor 2S2 - Homo |
| | | | | sapiens |
| NOV33c | SNP13382442 | 413 | 414 | Olfactory receptor 2S2 - Homo |
| | | | | sapiens |
| NOV33d | SNP13373880 | 415 | 416 | Olfactory receptor 2S2 - Homo |
| | | | | sapiens |
| NOV33e | SNP13373881 | 417 | 418 | Olfactory receptor 2S2 - Homo |
| | | | | sapiens |
| NOV33f | SNP13382443 | 419 | 420 | Olfactory receptor 2S2 - Homo |
| | | | | sapiens |
| NOV33g | SNP13373844 | 421 | 422 | Olfactory receptor 2S2 - Homo |
| | | | | sapiens |
| NOV33h | SNP13373883 | 423 | 424 | Olfactory receptor 2S2 - Homo |
| | | | | sapiens |
| NOV34a | CG54212-02 | 425 | 426 | Seven transmembrane helix receptor - |
| | | | | Homo sapiens |
| NOV34b | CG54212-01 | 427 | 428 | Seven transmembrane helix receptor - |
| | | | | Homo sapiens |
| NOV34c | CG54212-03 | 429 | 430 | Seven transmembrane helix receptor - |
| | | | | Homo sapiens |
| NOV34d | CG54212-04 | 431 | 432 | Seven transmembrane helix receptor - |
| | | | | Homo sapiens |
| NOV34e | SNP13373981 | 433 | 434 | Seven transmembrane helix receptor - |
| | | | | Homo sapiens |
| NOV34f | SNP13373982 | 435 | 436 | Seven transmembrane helix receptor - |
| | | | | Homo sapiens |
| NOV34g | SNP13373984 | 437 | 438 | Seven transmembrane helix receptor - |
| | | | | Homo sapiens |
| NOV34h | SNP13373985 | 439 | 440 | Seven transmembrane helix receptor - |
| | | | | Homo sapiens |
| NOV35a | CG54236-01 | 441 | 442 | Cysteinyl leukotriene receptor 2 |
| | | | | (CysLTR2) (PSEC0146) (HG57) |
| | | | | (HPN321) (hGPCR21) - Homo |
| | | | | sapiens |
| NOV35b | CG54236-02 | 443 | 444 | Cysteinyl leukotriene receptor 2 |
| | | | | (CysLTR2) (PSEC0146) (HG57) |
| | | | | (HPN321) (hGPCR21) - Homo |
| | | | | sapiens |
| NOV36a | CG54479-06 | 445 | 446 | Macrophage stimulating 1 |
| | | | | (Hepatocyte growth factor-like) - |
| | | | | Homo sapiens |
| NOV36b | CG54479-05 | 447 | 448 | Macrophage stimulating 1 |
| | | | | (Hepatocyte growth factor-like) - |
| | | | | Homo sapiens |
| NOV36c | CG54479-01 | 449 | 450 | Macrophage stimulating 1 |
| | | | | (Hepatocyte growth factor-like) - |
| | | | | Homo sapiens |
| NOV36d | CG54479-02 | 451 | 452 | Macrophage stimulating 1 |
| | | | | (Hepatocyte growth factor-like) - |
| | | | | Homo sapiens |
| NOV36e | CG54479-03 | 453 | 454 | Macrophage stimulating 1 |
| | | | | (Hepatocyte growth factor-like) - |
| | | | | Homo sapiens |
| NOV36f | CG54479-04 | 455 | 456 | Macrophage stimulating 1 |
| | | | | (Hepatocyte growth factor-like) - |
| | | | | Homo sapiens |
| NOV37a | CG54539-02 | 457 | 458 | Zinc transporter 1 (ZnT-1) - Homo |
| | | | | sapiens |
| NOV37b | CG54539-01 | 459 | 460 | Zinc transporter 1 (ZnT-1) - Homo |
| | | | | sapiens |
| NOV37c | SNP13382438 | 461 | 462 | Zinc transporter 1 (ZnT-1) - Homo |
| | | | | sapiens |
| NOV38a | CG54683-05 | 463 | 464 | Gamma-aminobutyric-acid receptor |
| | | | | rho-3 subunit precursor (GABA(A) |
| | | | | receptor) - Rattus norvegicus |
| NOV38b | CG54683-01 | 465 | 466 | Gamma-aminobutyric-acid receptor |
| | | | | rho-3 subunit precursor (GABA(A) |
| | | | | receptor) - Rattus norvegicus |
| NOV38c | CG54683-02 | 467 | 468 | Gamma-aminobutyric-acid receptor |
| | | | | rho-3 subunit precursor (GABA(A) |
| | | | | receptor) - Rattus norvegicus |
| NOV38d | CG54683-03 | 469 | 470 | Gamma-aminobutyric-acid receptor |
| | | | | rho-3 subunit precursor (GABA(A) |
| | | | | receptor) - Rattus norvegicus |
| NOV38e | CG54683-04 | 471 | 472 | Gamma-aminobutyric-acid receptor |
| | | | | rho-3 subunit precursor (GABA(A) |
| | | | | receptor) - Rattus norvegicus |
| NOV39a | CG54692-06 | 473 | 474 | Human G-protein coupled receptor |
| | | | | (GCREC-15) |
| NOV39b | CG54692-01 | 475 | 476 | Human G-protein coupled receptor |
| | | | | (GCREC-15) |
| NOV39c | CG54692-02 | 477 | 478 | Human G-protein coupled receptor |
| | | | | (GCREC-15) |
| NOV39d | CG54692-03 | 479 | 480 | Human G-protein coupled receptor |
| | | | | (GCREC-15) |
| NOV39e | CG54692-04 | 481 | 482 | Human G-protein coupled receptor |
| | | | | (GCREC-15) |
| NOV39f | CG54692-05 | 483 | 484 | Human G-protein coupled receptor |
| | | | | (GCREC-15) |
| NOV40a | CG55069-01 | 485 | 486 | Ten-m3 - Mus musculus |
| NOV40b | CG55069-04 | 487 | 488 | Ten-m3 - Mus musculus |
| NOV40c | 248993047 | 489 | 490 | Ten-m3 - Mus musculus |
| NOV40d | 262802488 | 491 | 492 | Ten-m3 - Mus musculus |
| NOV40e | 248993606 | 493 | 494 | Ten-m3 - Mus musculus |
| NOV40f | 314411758 | 495 | 496 | Ten-m3 - Mus musculus |
| NOV40g | 319067006 | 497 | 498 | Ten-m3 - Mus musculus |
| NOV40h | 319506086 | 499 | 500 | Ten-m3 - Mus musculus |
| NOV40i | CG55069-03 | 501 | 502 | Ten-m3 - Mus musculus |
| NOV40j | 219937039 | 503 | 504 | Ten-m3 - Mus musculus |
| NOV40k | 219937046 | 505 | 506 | Ten-m3 - Mus musculus |
| NOV40l | 219937583 | 507 | 508 | Ten-m3 - Mus musculus |
| NOV40m | 219937000 | 509 | 510 | Ten-m3 - Mus musculus |
| NOV40n | 219937005 | 511 | 512 | Ten-m3 - Mus musculus |
| NOV40o | 219937013 | 513 | 514 | Ten-m3 - Mus musculus |
| NOV40p | 219937063 | 515 | 516 | Ten-m3 - Mus musculus |
| NOV40q | CG55069-09 | 517 | 518 | Ten-m3 - Mus musculus |
| NOV40r | 309327410 | 519 | 520 | Ten-m3 - Mus musculus |
| NOV40s | CG55069-02 | 521 | 522 | Ten-m3 - Mus musculus |
| NOV40t | CG55069-05 | 523 | 524 | Ten-m3 - Mus musculus |
| NOV40u | CG55069-06 | 525 | 526 | Ten-m3 - Mus musculus |
| NOV40v | CG55069-07 | 527 | 528 | Ten-m3 - Mus musculus |
| NOV40w | CG55069-08 | 529 | 530 | Ten-m3 - Mus musculus |
| NOV40x | CG55069-10 | 531 | 532 | Ten-m3 - Mus musculus |
| NOV40y | CG55069-11 | 533 | 534 | Ten-m3 - Mus musculus |
| NOV40z | CG55069-12 | 535 | 536 | Ten-m3 - Mus musculus |
| NOV40a | CG55069-13 | 537 | 538 | Ten-m3 - Mus musculus |
| NOV40b | CG55069-14 | 539 | 540 | Ten-m3 - Mus musculus |
| NOV40c | CG55069-15 | 541 | 542 | Ten-m3 - Mus musculus |
| NOV40d | SNP13374479 | 543 | 544 | Ten-m3 - Mus musculus |
| NOV40e | SNP13382453 | 545 | 546 | Ten-m3 - Mus musculus |
| NOV40f | SNP13382454 | 547 | 548 | Ten-m3 - Mus musculus |
| NOV40g | SNP13382455 | 549 | 550 | Ten-m3 - Mus musculus |
| NOV40h | SNP13378354 | 551 | 552 | Ten-m3 - Mus musculus |
| NOV41a | CG55343-03 | 553 | 554 | Olfactory receptor 2B6 (Hs6M1-32) |
| | | | | (Olfactory receptor 6-31) (OR6-31) - |
| | | | | Homo sapiens |
| NOV41b | 260568382 | 555 | 556 | Olfactory receptor 2B6 (Hs6M1-32) |
| | | | | (Olfactory receptor 6-31) (OR6-31) - |
| | | | | Homo sapiens |
| NOV41c | 314361391 | 557 | 558 | Olfactory receptor 2B6 (Hs6M1-32) |
| | | | | (Olfactory receptor 6-31) (OR6-31) - |
| | | | | Homo sapiens |
| NOV41d | 317286137 | 559 | 560 | Olfactory receptor 2B6 (Hs6M1-32) |
| | | | | (Olfactory receptor 6-31) (OR6-31) - |
| | | | | Homo sapiens |
| NOV41e | CG55343-01 | 561 | 562 | Olfactory receptor 2B6 (Hs6M1-32) |
| | | | | (Olfactory receptor 6-31) (OR6-31) - |
| | | | | Homo sapiens |
| NOV41f | CG55343-02 | 563 | 564 | Olfactory receptor 2B6 (Hs6M1-32) |
| | | | | (Olfactory receptor 6-31) (OR6-31) - |
| | | | | Homo sapiens |
| NOV41g | CG55343-04 | 565 | 566 | Olfactory receptor 2B6 (Hs6M1-32) |
| | | | | (Olfactory receptor 6-31) (OR6-31) - |
| | | | | Homo sapiens |
| NOV41h | CG55343-05 | 567 | 568 | Olfactory receptor 2B6 (Hs6M1-32) |
| | | | | (Olfactory receptor 6-31) (OR6-31) - |
| | | | | Homo sapiens |
| NOV41i | CG55343-06 | 569 | 570 | Olfactory receptor 2B6 (Hs6M1-32) |
| | | | | (Olfactory receptor 6-31) (OR6-31) - |
| | | | | Homo sapiens |
| NOV41j | SNP13373740 | 571 | 572 | Olfactory receptor 2B6 (Hs6M1-32) |
| | | | | (Olfactory receptor 6-31) (OR6-31) - |
| | | | | Homo sapiens |
| NOV41k | SNP13376425 | 573 | 574 | Olfactory receptor 2B6 (Hs6M1-32) |
| | | | | (Olfactory receptor 6-31) (OR6-31) - |
| | | | | Homo sapiens |
| NOV41l | SNP13376424 | 575 | 576 | Olfactory receptor 2B6 (Hs6M1-32) |
| | | | | (Olfactory receptor 6-31) (OR6-31) - |
| | | | | Homo sapiens |
| NOV41m | SNP13376423 | 577 | 578 | Olfactory receptor 2B6 (Hs6M1-32) |
| | | | | (Olfactory receptor 6-31) (OR6-31) - |
| | | | | Homo sapiens |
| NOV42a | CG55358-04 | 579 | 580 | G-protein coupled receptor 4a |
| | | | | (GPCR4a) |
| NOV42b | CG55358-03 | 581 | 582 | G-protein coupled receptor 4a |
| | | | | (GPCR4a) |
| NOV42c | 317863291 | 583 | 584 | G-protein coupled receptor 4a |
| | | | | (GPCR4a) |
| NOV42d | 317863328 | 585 | 586 | G-protein coupled receptor 4a |
| | | | | (GPCR4a) |
| NOV42e | 317863350 | 587 | 588 | G-protein coupled receptor 4a |
| | | | | (GPCR4a) |
| NOV42f | 271624076 | 589 | 590 | G-protein coupled receptor 4a |
| | | | | (GPCR4a) |
| NOV42g | CG55358-01 | 591 | 592 | G-protein coupled receptor 4a |
| | | | | (GPCR4a) |
| NOV42h | CG55358-02 | 593 | 594 | G-protein coupled receptor 4a |
| | | | | (GPCR4a) |
| NOV42i | CG55358-05 | 595 | 596 | G-protein coupled receptor 4a |
| | | | | (GPCR4a) |
| NOV42j | CG55358-06 | 597 | 598 | G-protein coupled receptor 4a |
| | | | | (GPCR4a) |
| NOV42k | CG55358-07 | 599 | 600 | G-protein coupled receptor 4a |
| | | | | (GPCR4a) |
| NOV42l | SNP13375122 | 601 | 602 | G-protein coupled receptor 4a |
| | | | | (GPCR4a) |
| NOV42m | SNP13375123 | 603 | 604 | G-protein coupled receptor 4a |
| | | | | (GPCR4a) |
| NOV42n | SNP13382494 | 605 | 606 | G-protein coupled receptor 4a |
| | | | | (GPCR4a) |
| NOV42o | SNP13375124 | 607 | 608 | G-protein coupled receptor 4a |
| | | | | (GPCR4a) |
| NOV42p | SNP13375125 | 609 | 610 | G-protein coupled receptor 4a |
| | | | | (GPCR4a) |
| NOV42q | SNP13376426 | 611 | 612 | G-protein coupled receptor 4a |
| | | | | (GPCR4a) |
| NOV42r | SNP13376427 | 613 | 614 | G-protein coupled receptor 4a |
| | | | | (GPCR4a) |
| NOV42s | SNP13375126 | 615 | 616 | G-protein coupled receptor 4a |
| | | | | (GPCR4a) |
| NOV43a | CG55604-04 | 617 | 618 | Seven transmembrane helix receptor |
| NOV43b | CG55604-01 | 619 | 620 | Seven transmembrane helix receptor |
| NOV43c | CG55604-02 | 621 | 622 | Seven transmembrane helix receptor |
| NOV43d | CG55604-03 | 623 | 624 | Seven transmembrane helix receptor |
| NOV43e | CG55604-05 | 625 | 626 | Seven transmembrane helix receptor |
| NOV43f | CG55604-06 | 627 | 628 | Seven transmembrane helix receptor |
| NOV43g | CG55604-07 | 629 | 630 | Seven transmembrane helix receptor |
| NOV43h | SNP13019742 | 631 | 632 | Seven transmembrane helix receptor |
| NOV43i | SNP13373776 | 633 | 634 | Seven transmembrane helix receptor |
| NOV43j | SNP13373777 | 635 | 636 | Seven transmembrane helix receptor |
| NOV43k | SNP13373778 | 637 | 638 | Seven transmembrane helix receptor |
| N0V43l | SNP13373838 | 639 | 640 | Seven transmembrane helix receptor |
| NOV43m | SNP13373837 | 641 | 642 | Seven transmembrane helix receptor |
| NOV43n | SNP13373836 | 643 | 644 | Seven transmembrane helix receptor |
| NOV43o | SNP13373780 | 645 | 646 | Seven transmembrane helix receptor |
| NOV43p | SNP13373781 | 647 | 648 | Seven transmembrane helix receptor |
| NOV43q | SNP13373782 | 649 | 650 | Seven transmembrane helix receptor |
| NOV43r | SNP13373783 | 651 | 652 | Seven transmembrane helix receptor |
| NOV43s | SNP13373833 | 653 | 654 | Seven transmembrane helix receptor |
| NOV43t | SNP13373784 | 655 | 656 | Seven transmembrane helix receptor |
| NOV43u | SNP13373832 | 657 | 658 | Seven transmembrane helix receptor |
| NOV44a | CG55752-07 | 659 | 660 | Glucosidase - Homo sapiens |
| | | | | (Human), 769 aa |
| NOV44b | CG55752-06 | 661 | 662 | Glucosidase - Homo sapiens |
| | | | | (Human), 769 aa |
| NOV44c | CG55752-01 | 663 | 664 | Glucosidase - Homo sapiens |
| | | | | (Human), 769 aa |
| NOV44d | CG55752-02 | 665 | 666 | Glucosidase - Homo sapiens |
| | | | | (Human), 769 aa |
| NOV44e | CG55752-03 | 667 | 668 | Glucosidase - Homo sapiens |
| | | | | (Human), 769 aa |
| NOV44f | CG55752-04 | 669 | 670 | Glucosidase - Homo sapiens |
| | | | | (Human), 769 aa |
| NOV44g | CG55752-05 | 671 | 672 | Glucosidase - Homo sapiens |
| | | | | (Human), 769 aa |
| NOV44h | SNP13379656 | 673 | 674 | Glucosidase - Homo sapiens |
| | | | | (Human), 769 aa |
| NOV44i | SNP13379655 | 675 | 676 | Glucosidase - Homo sapiens |
| | | | | (Human), 769 aa |
| NOV44j | SNP13379654 | 677 | 678 | Glucosidase - Homo sapiens |
| | | | | (Human), 769 aa |
| NOV45a | CG55778-03 | 679 | 680 | Aldo-keto reductase related protein 3 - |
| | | | | Homo sapiens |
| NOV45b | CG55778-06 | 681 | 682 | Aldo-keto reductase related protein 3 - |
| | | | | Homo sapiens |
| NOV45c | 275480984 | 683 | 684 | Aldo-keto reductase related protein 3 - |
| | | | | Homo sapiens |
| NOV45d | CG55778-01 | 685 | 686 | Aldo-keto reductase related protein 3 - |
| | | | | Homo sapiens |
| NOV45e | CG55778-02 | 687 | 688 | Aldo-keto reductase related protein 3 - |
| | | | | Homo sapiens |
| NOV45f | CG55778-04 | 689 | 690 | Aldo-keto reductase related protein 3 - |
| | | | | Homo sapiens |
| NOV45g | CG55778-05 | 691 | 692 | Aldo-keto reductase related protein 3 - |
| | | | | Homo sapiens |
| NOV45h | CG55778-07 | 693 | 694 | Aldo-keto reductase related protein 3 - |
| | | | | Homo sapiens |
| NOV45i | CG55778-08 | 695 | 696 | Aldo-keto reductase related protein 3 - |
| | | | | Homo sapiens |
| NOV45j | SNP13375813 | 697 | 698 | Aldo-keto reductase related protein 3 - |
| | | | | Homo sapiens |
| NOV46a | CG55794-03 | 699 | 700 | Zn-carboxypeptidase - Homo sapiens |
| NOV46b | 210223559 | 701 | 702 | Zn-carboxypeptidase - Homo sapiens |
| NOV46c | 210223626 | 703 | 704 | Zn-carboxypeptidase - Homo sapiens |
| NOV46d | 171095097 | 705 | 706 | Zn-carboxypeptidase - Homo sapiens |
| NOV46e | 183852229 | 707 | 708 | Zn-carboxypeptidase - Homo sapiens |
| NOV46f | 183852264 | 709 | 710 | Zn-carboxypeptidase - Homo sapiens |
| NOV46g | 183852410 | 711 | 712 | Zn-carboxypeptidase - Homo sapiens |
| NOV46h | 183523337 | 713 | 714 | Zn-carboxypeptidase - Homo sapiens |
| NOV46i | CG55794-01 | 715 | 716 | Zn-carboxypeptidase - Homo sapiens |
| NOV46j | CG55794-02 | 717 | 718 | Zn-carboxypeptidase - Homo sapiens |
| NOV46k | CG55794-04 | 719 | 720 | Zn-carboxypeptidase - Homo sapiens |
| NOV46l | CG55794-05 | 721 | 722 | Zn-carboxypeptidase - Homo sapiens |
| NOV46m | CG55794-06 | 723 | 724 | Zn-carboxypeptidase - Homo sapiens |
| NOV46n | CG55794-07 | 725 | 726 | Zn-carboxypeptidase - Homo sapiens |
| NOV46o | CG55794-08 | 727 | 728 | Zn-carboxypeptidase - Homo sapiens |
| NOV46p | CG55794-09 | 729 | 730 | Zn-carboxypeptidase - Homo sapiens |
| NOV46q | CG55794-10 | 731 | 732 | Zn-carboxypeptidase - Homo sapiens |
| NOV46r | CG55794-11 | 733 | 734 | Zn-carboxypeptidase - Homo sapiens |
| NOV46s | CG55794-12 | 735 | 736 | Zn-carboxypeptidase - Homo sapiens |
| NOV46t | CG55794-13 | 737 | 738 | Zn-carboxypeptidase - Homo sapiens |
| NOV46u | SNP13375362 | 739 | 740 | Zn-carboxypeptidase - Homo sapiens |
| NOV46v | SNP13379598 | 741 | 742 | Zn-carboxypeptidase - Homo sapiens |
| NOV46w | SNP13375066 | 743 | 744 | Zn-carboxypeptidase - Homo sapiens |
| NOV46x | SNP13375067 | 745 | 746 | Zn-carboxypeptidase - Homo sapiens |
| NOV47a | CG55806-04 | 747 | 748 | FACTOR IX - Homo sapiens |
| NOV47b | CG55806-01 | 749 | 750 | FACTOR IX - Homo sapiens |
| NOV47c | CG55806-02 | 751 | 752 | FACTOR IX - Homo sapiens |
| NOV47d | CG55806-03 | 753 | 754 | FACTOR IX - Homo sapiens |
| NOV47e | SNP13382503 | 755 | 756 | FACTOR IX - Homo sapiens |
| NOV47f | SNP13382492 | 757 | 758 | FACTOR IX - Homo sapiens |
| NOV48a | CG55828-02 | 759 | 760 | Serine/threonine-protein kinase PAK |
| | | | | 7 (EC 2.7.1.—) (p21-activated kinase 7) |
| | | | | (PAK-7) (PAK-5) - Homo sapiens |
| NOV48b | CG55828-01 | 761 | 762 | Serine/threonine-protein kinase PAK |
| | | | | 7 (EC 2.7.1.—) (p21-activated kinase 7) |
| | | | | (PAK-7) (PAK-5) - Homo sapiens |
| NOV48c | SNP13379517 | 763 | 764 | Serine/threonine-protein kinase PAK |
| | | | | 7 (EC 2.7.1.—) (p21-activated kinase 7) |
| | | | | (PAK-7) (PAK-5) - Homo sapiens |
| NOV48d | SNP13376535 | 765 | 766 | Serine/threonine-protein kinase PAK |
| | | | | 7 (EC 2.7.1.—) (p21-activated kinase 7) |
| | | | | (PAK-7) (PAK-5) - Homo sapiens |
| NOV48e | SNP13382500 | 767 | 768 | Serine/threonine-protein kinase PAK |
| | | | | 7 (EC 2.7.1.—) (p21-activated kinase 7) |
| | | | | (PAK-7) (PAK-5) - Homo sapiens |
| NOV48f | SNP13375705 | 769 | 770 | Serine/threonine-protein kinase PAK |
| | | | | 7 (EC 2.7.1.—) (p21-activated kinase 7) |
| | | | | (PAK-7) (PAK-5) - Homo sapiens |
| NOV49a | CG55980-02 | 771 | 772 | Seven transmembrane helix receptor - |
| | | | | Homo sapiens |
| NOV49b | CG55980-01 | 773 | 774 | Seven transmembrane helix receptor - |
| | | | | Homo sapiens |
| NOV50a | CG55988-03 | 775 | 776 | Organic cation transporter OKB1 - |
| | | | | Homo sapiens |
| NOV50b | CG55988-04 | 777 | 778 | Organic cation transporter OKB1 - |
| | | | | Homo sapiens |
| NOV50c | CG55988-01 | 779 | 780 | Organic cation transporter OKB1 - |
| | | | | Homo sapiens |
| NOV50d | CG55988-02 | 781 | 782 | Organic cation transporter OKB1 - |
| | | | | Homo sapiens |
| NOV51a | CG56071-01 | 783 | 784 | Mixed lineage kinase MLK1 - Homo |
| | | | | sapiens (Human), 1066 aa |
| NOV51b | 274082270 | 785 | 786 | Mixed lineage kinase MLK1 - Homo |
| | | | | sapiens (Human), 1066 aa |
| NOV51c | SNP13376041 | 787 | 788 | Mixed lineage kinase MLK1 - Homo |
| | | | | sapiens (Human), 1066 aa |
| NOV52a | CG56142-01 | 789 | 790 | Similar to protease, serine, 8 |
| | | | | (Prostasin) - Homo sapiens (Human), |
| | | | | 327 aa |
| NOV52b | CG56142-04 | 791 | 792 | Similar to protease, serine, 8 |
| | | | | (Prostasin) - Homo sapiens (Human), |
| | | | | 327 aa |
| NOV52c | 276873337 | 793 | 794 | Similar to protease, serine, 8 |
| | | | | (Prostasin) - Homo sapiens (Human), |
| | | | | 327 aa |
| NOV52d | 276863970 | 795 | 796 | Similar to protease, serine, 8 |
| | | | | (Prostasin) - Homo sapiens (Human), |
| | | | | 327 aa |
| NOV52e | 276863992 | 797 | 798 | Similar to protease, serine, 8 |
| | | | | (Prostasin) - Homo sapiens (Human), |
| | | | | 327 aa |
| NOV52f | 276873330 | 799 | 800 | Similar to protease, serine, 8 |
| | | | | (Prostasin) - Homo sapiens (Human), |
| | | | | 327 aa |
| NOV52g | CG56142-02 | 801 | 802 | Similar to protease, serine, 8 |
| | | | | (Prostasin) - Homo sapiens (Human), |
| | | | | 327 aa |
| NOV52h | CG56142-03 | 803 | 804 | Similar to protease, serine, 8 |
| | | | | (Prostasin) - Homo sapiens (Human), |
| | | | | 327 aa |
| NOV52i | CG56142-05 | 805 | 806 | Similar to protease, serine, 8 |
| | | | | (Prostasin) - Homo sapiens (Human), |
| | | | | 327 aa |
| NOV52j | CG56142-06 | 807 | 808 | Similar to protease, serine, 8 |
| | | | | (Prostasin) - Homo sapiens (Human), |
| | | | | 327 aa |
| NOV53a | CG56144-01 | 809 | 810 | Seven transmembrane helix receptor - |
| | | | | Homo sapiens |
| NOV53b | 170645965 | 811 | 812 | Seven transmembrane helix receptor - |
| | | | | Homo sapiens |
| NOV53c | 168869277 | 813 | 814 | Seven transmembrane helix receptor - |
| | | | | Homo sapiens |
| NOV53d | 170645981 | 815 | 816 | Seven transmembrane helix receptor - |
| | | | | Homo sapiens |
| NOV53e | 168869262 | 817 | 818 | Seven transmembrane helix receptor - |
| | | | | Homo sapiens |
| NOV53f | 168869254 | 819 | 820 | Seven transmembrane helix receptor - |
| | | | | Homo sapiens |
| NOV53g | CG56144-02 | 821 | 822 | Seven transmembrane helix receptor - |
| | | | | Homo sapiens |
| NOV53h | CG56144-03 | 823 | 824 | Seven transmembrane helix receptor - |
| | | | | Homo sapiens |
| NOV53i | CG56144-04 | 825 | 826 | Seven transmembrane helix receptor - |
| | | | | Homo sapiens |
| NOV53j | CG56144-05 | 827 | 828 | Seven transmembrane helix receptor - |
| | | | | Homo sapiens |
| NOV53k | CG56144-06 | 829 | 830 | Seven transmembrane helix receptor - |
| | | | | Homo sapiens |
| NOV54a | CG56146-02 | 831 | 832 | Seven transmembrane helix receptor - |
| | | | | Homo sapiens |
| NOV54b | CG56146-01 | 833 | 834 | Seven transmembrane helix receptor - |
| | | | | Homo sapiens |
| NOV54c | 170646057 | 835 | 836 | Seven transmembrane helix receptor - |
| | | | | Homo sapiens |
| NOV54d | 170646049 | 837 | 838 | Seven transmembrane helix receptor - |
| | | | | Homo sapiens |
| NOV54e | 170646053 | 839 | 840 | Seven transmembrane helix receptor - |
| | | | | Homo sapiens |
| NOV54f | 174307717 | 841 | 842 | Seven transmembrane helix receptor - |
| | | | | Homo sapiens |
| NOV54g | 168869383 | 843 | 844 | Seven transmembrane helix receptor - |
| | | | | Homo sapiens |
| NOV54h | CG56146-03 | 845 | 846 | Seven transmembrane helix receptor - |
| | | | | Homo sapiens |
| NOV54i | 262939640 | 847 | 848 | Seven transmembrane helix receptor - |
| | | | | Homo sapiens |
| NOV54j | CG56146-04 | 849 | 850 | Seven transmembrane helix receptor - |
| | | | | Homo sapiens |
| NOV54k | CG56146-05 | 851 | 852 | Seven transmembrane helix receptor - |
| | | | | Homo sapiens |
| NOV54l | CG56146-06 | 853 | 854 | Seven transmembrane helix receptor - |
| | | | | Homo sapiens |
| NOV55a | CG56258-04 | 855 | 856 | Na exchanger isoform 3 splice variant |
| | | | | 2 - Homo sapiens |
| NOV55b | CG56258-02 | 857 | 858 | Na exchanger isoform 3 splice variant |
| | | | | 2 - Homo sapiens |
| NOV55c | 258076220 | 859 | 860 | Na exchanger isoform 3 splice variant |
| | | | | 2 - Homo sapiens |
| NOV55d | 248057963 | 861 | 862 | Na exchanger isoform 3 splice variant |
| | | | | 2 - Homo sapiens |
| NOV55e | CG56258-01 | 863 | 864 | Na exchanger isoform 3 splice variant |
| | | | | 2 - Homo sapiens |
| NOV55f | CG56258-03 | 865 | 866 | Na exchanger isoform 3 splice variant |
| | | | | 2 - Homo sapiens |
| NOV55g | CG56258-05 | 867 | 868 | Na exchanger isoform 3 splice variant |
| | | | | 2 - Homo sapiens |
| NOV55h | CG56258-06 | 869 | 870 | Na exchanger isoform 3 splice variant |
| | | | | 2 - Homo sapiens |
| NOV56a | CG56262-01 | 871 | 872 | Putative calcium binding transporter - |
| | | | | Homo sapiens |
| NOV56b | 266120550 | 873 | 874 | Putative calcium binding transporter - |
| | | | | Homo sapiens |
| NOV57a | CG56315-01 | 875 | 876 | Connexin25 - Homo sapiens |
| NOV57b | 247679382 | 877 | 878 | Connexin25 - Homo sapiens |
| NOV57c | 247678321 | 879 | 880 | Connexin25 - Homo sapiens |
| NOV57d | 247679418 | 881 | 882 | Connexin25 - Homo sapiens |
| NOV57e | 247679395 | 883 | 884 | Connexin25 - Homo sapiens |
| NOV57f | 247679328 | 885 | 886 | Connexin25 - Homo sapiens |
| NOV57g | CG56315-02 | 887 | 888 | Connexin25 - Homo sapiens |
| NOV57h | CG56315-03 | 889 | 890 | Connexin25 - Homo sapiens |
| NOV57i | CG56315-04 | 891 | 892 | Connexin25 - Homo sapiens |
| NOV57j | CG56315-05 | 893 | 894 | Connexin25 - Homo sapiens |
| NOV57k | CG56315-06 | 895 | 896 | Connexin25 - Homo sapiens |
| NOV57l | CG56315-07 | 897 | 898 | Connexin25 - Homo sapiens |
| NOV57m | CG56315-08 | 899 | 900 | Connexin25 - Homo sapiens |
| NOV57n | SNP13381650 | 901 | 902 | Connexin25 - Homo sapiens |
| NOV57o | SNP13381651 | 903 | 904 | Connexin25 - Homo sapiens |
| NOV57p | SNP13381652 | 905 | 906 | Connexin25 - Homo sapiens |
| NOV57q | SNP13381653 | 907 | 908 | Connexin25 - Homo sapiens |
| NOV57r | SNP13381654 | 909 | 910 | Connexin25 - Homo sapiens |
| NOV58a | CG56398-01 | 911 | 912 | Sodium/glucose cotransporter KST1 - |
| | | | | Homo sapiens |
| NOV58b | 265726152 | 913 | 914 | Sodium/glucose cotransporter KST1 - |
| | | | | Homo sapiens |
| NOV58c | 267254040 | 915 | 916 | Sodium/glucose cotransporter KST1 - |
| | | | | Homo sapiens |
| NOV58d | SNP13379242 | 917 | 918 | Sodium/glucose cotransporter KST1 - |
| | | | | Homo sapiens |
| NOV59a | CG56605-03 | 919 | 920 | Protease PRTS-5 |
| NOV59b | CG56605-01 | 921 | 922 | Protease PRTS-5 |
| NOV59c | CG56605-02 | 923 | 924 | Protease PRTS-5 |
| NOV60a | CG56645-03 | 925 | 926 | sodium/glucose cotransporter |
| | | | | Fbh68723pat |
| NOV60b | CG56645-04 | 927 | 928 | sodium/glucose cotransporter |
| | | | | Fbh68723pat |
| NOV60c | CG56645-01 | 929 | 930 | sodium/glucose cotransporter |
| | | | | Fbh68723pat |
| NOV60d | CG56645-02 | 931 | 932 | sodium/glucose cotransporter |
| | | | | Fbh68723pat |
| NOV61a | CG56667-01 | 933 | 934 | Seven transmembrane helix receptor - |
| | | | | Homo sapiens |
| NOV61b | 268952113 | 935 | 936 | Seven transmembrane helix receptor - |
| | | | | Homo sapiens |
| NOV62a | CG56868-01 | 937 | 938 | ADAM 7 precursor (A disintegrin and |
| | | | | metalloproteinase domain 7) (Sperm |
| | | | | maturation-related glycoprotein GP- |
| | | | | 83) - Homo sapiens |
| NOV62b | 276580332 | 939 | 940 | ADAM 7 precursor (A disintegrin and |
| | | | | metalloproteinase domain 7) (Sperm |
| | | | | maturation-related glycoprotein GP- |
| | | | | 83) - Homo sapiens |
| NOV63a | CG56870-01 | 941 | 942 | NDRG3 protein - Homo sapiens |
| NOV63b | CG56870-06 | 943 | 944 | NDRG3 protein - Homo sapiens |
| NOV63c | 276585681 | 945 | 946 | NDRG3 protein - Homo sapiens |
| NOV63d | CG56870-02 | 947 | 948 | NDRG3 protein - Homo sapiens |
| NOV63e | CG56870-03 | 949 | 950 | NDRG3 protein - Homo sapiens |
| NOV63f | CG56870-04 | 951 | 952 | NDRG3 protein - Homo sapiens |
| NOV63g | CG56870-05 | 953 | 954 | NDRG3 protein - Homo sapiens |
| NOV64a | CG57109-01 | 955 | 956 | Human kinase (PKIN)-4 |
| NOV64b | 260457400 | 957 | 958 | Human kinase (PKIN)-4 |
| NOV64c | 260457409 | 959 | 960 | Human kinase (PKIN)-4 |
| NOV64d | CG57109-05 | 961 | 962 | Human kinase (PKIN)-4 |
| NOV64e | 267253965 | 963 | 964 | Human kinase (PKIN)-4 |
| NOV64f | 267254000 | 965 | 966 | Human kinase (PKIN)-4 |
| NOV64g | 267253987 | 967 | 968 | Human kinase (PKIN)-4 |
| NOV64h | CG57109-02 | 969 | 970 | Human kinase (PKIN)-4 |
| NOV64i | CG57109-03 | 971 | 972 | Human kinase (PKIN)-4 |
| NOV64j | CG57109-04 | 973 | 974 | Human kinase (PKIN)-4 |
| NOV64k | CG57109-06 | 975 | 976 | Human kinase (PKIN)-4 |
| NOV65a | CG57399-04 | 977 | 978 | Human phospholipase-like enzyme |
| NOV65b | CG57399-01 | 979 | 980 | Human phospholipase-like enzyme |
| NOV65c | CG57399-02 | 981 | 982 | Human phospholipase-like enzyme |
| NOV65d | CG57399-03 | 983 | 984 | Human phospholipase-like enzyme |
| NOV66a | CG57562-02 | 985 | 986 | cation-transporting ATPase 2 (EC |
| | | | | 3.6.3.—) (CGI-152) - Homo sapiens |
| NOV66b | CG57562-01 | 987 | 988 | cation-transporting ATPase 2 (EC |
| | | | | 3.6.3.—) (CGI-152) - Homo sapiens |
| NOV66c | SNP13380762 | 989 | 990 | cation-transporting ATPase 2 (EC |
| | | | | 3.6.3.—) (CGI-152) - Homo sapiens |
| NOV66d | SNP13380787 | 991 | 992 | cation-transporting ATPase 2 (EC |
| | | | | 3.6.3.—) (CGI-152) - Homo sapiens |
| NOV67a | CG57758-03 | 993 | 994 | Na+coupled citrate transporter protein - |
| | | | | Homo sapiens |
| NOV67b | 308537854 | 995 | 996 | Na+coupled citrate transporter protein - |
| | | | | Homo sapiens |
| NOV67c | CG57758-01 | 997 | 998 | Na+coupled citrate transporter protein - |
| | | | | Homo sapiens |
| NOV67d | CG57758-02 | 999 | 1000 | Na+coupled citrate transporter protein - |
| | | | | Homo sapiens |
| NOV67e | CG57758-04 | 1001 | 1002 | Na+coupled citrate transporter protein - |
| | | | | Homo sapiens |
| NOV67f | CG57758-05 | 1003 | 1004 | Na+coupled citrate transporter protein - |
| | | | | Homo sapiens |
| NOV68a | CG58504-01 | 1005 | 1006 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68b | 169648376 | 1007 | 1008 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68c | 169648388 | 1009 | 1010 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68d | 169648365 | 1011 | 1012 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68e | 284068250 | 1013 | 1014 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68f | 305867866 | 1015 | 1016 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68g | 318176397 | 1017 | 1018 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68h | CG58504-02 | 1019 | 1020 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68i | CG58504-03 | 1021 | 1022 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68j | CG58504-04 | 1023 | 1024 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68k | CG58504-05 | 1025 | 1026 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68l | CG58504-06 | 1027 | 1028 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV68m | CG58504-07 | 1029 | 1030 | ADAMTS-12 precursor (EC 3.4.24.—) |
| | | | | (A disintegrin and metalloproteinase |
| | | | | with thrombospondin motifs 12) |
| | | | | (ADAM-TS 12) (ADAM-TS12) - |
| | | | | Homo sapiens |
| NOV69a | CG58510-01 | 1031 | 1032 | Cation transport protein YRDO |
| NOV70a | CG59309-01 | 1033 | 1034 | Human Acyl-CoA thioesterase |
| NOV70b | 278901386 | 1035 | 1036 | Human Acyl-CoA thioesterase |
| NOV71a | CG59490-01 | 1037 | 1038 | Mast cell protease-11 - Mus musculus |
| NOV71b | 207639512 | 1039 | 1040 | Mast cell protease-11 - Mus musculus |
| NOV71c | 207639476 | 1041 | 1042 | Mast cell protease-11 - Mus musculus |
| NOV71d | 207639523 | 1043 | 1044 | Mast cell protease-11 - Mus musculus |
| NOV71e | CG59490-02 | 1045 | 1046 | Mast cell protease-11 - Mus musculus |
| NOV72a | CG59693-01 | 1047 | 1048 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72b | CG59693-03 | 1049 | 1050 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72c | 277637252 | 1051 | 1052 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72d | CG59693-02 | 1053 | 1054 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | dehydrogenase) - Homo sapiens |
| NOV72e | CG59693-04 | 1055 | 1056 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72f | CG59693-05 | 1057 | 1058 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72g | CG59693-06 | 1059 | 1060 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72h | CG59693-07 | 1061 | 1062 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72i | CG59693-08 | 1063 | 1064 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV72j | CG59693-09 | 1065 | 1066 | Aldo-keto reductase family 1 member |
| | | | | C1 (EC 1.1.1.—) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV73a | CG59839-02 | 1067 | 1068 | cation-transporting ATPase 3 (EC |
| | | | | 3.6.3.—) (ATPase family homolog up- |
| | | | | regulated in senescence cells 1) - |
| | | | | Homo sapiens |
| NOV73b | CG59839-01 | 1069 | 1070 | cation-transporting ATPase 3 (EC |
| | | | | 3.6.3.—) (ATPase family homolog up- |
| | | | | regulated in senescence cells 1) - |
| | | | | Homo sapiens |
| NOV74a | CG90866-04 | 1071 | 1072 | Human protein kinase |
| NOV74b | CG90866-03 | 1073 | 1074 | Human protein kinase |
| NOV74c | CG90866-01 | 1075 | 1076 | Human protein kinase |
| NOV74d | CG90866-02 | 1077 | 1078 | Human protein kinase |
| NOV74e | CG90866-05 | 1079 | 1080 | Human protein kinase |
| NOV75a | CG91708-02 | 1081 | 1082 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV75b | 262751856 | 1083 | 1084 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV75c | CG91708-01 | 1085 | 1086 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV75d | CG91708-03 | 1087 | 1088 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV75e | CG91708-04 | 1089 | 1090 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV75f | SNP13380740 | 1091 | 1092 | Stromelysin-1 precursor (EC |
| | | | | 3.4.24.17) (Matrix metalloproteinase- |
| | | | | 3) (MMP-3) (Transin-1) (SL-1) - |
| | | | | Homo sapiens |
| NOV76a | CG92078-02 | 1093 | 1094 | Yolk SAC permease-like YSPL-1 |
| | | | | form 1 (Yolk SAC permease-like |
| | | | | YSPL-1 form 4) (Yolk SAC |
| | | | | permease-like YSPL-1 form 3) (Yolk |
| | | | | SAC permease-like YSPL-1 form 2) - |
| | | | | Mus musculus |
| NOV76b | CG92078-01 | 1095 | 1096 | Yolk SAC permease-like YSPL-1 |
| | | | | form 1 (Yolk SAC permease-like |
| | | | | YSPL-1 form 4) (Yolk SAC |
| | | | | permease-like YSPL-1 form 3) (Yolk |
| | | | | SAC permease-like YSPL-1 form 2) - |
| | | | | Mus musculus |
| NOV77a | CG93669-04 | 1097 | 1098 | NIMA-related protein kinase 3 - |
| | | | | Homo sapiens |
| NOV77b | CG93669-01 | 1099 | 1100 | NIMA-related protein kinase 3 - |
| | | | | Homo sapiens |
| NOV77c | CG93669-02 | 1101 | 1102 | NIMA-related protein kinase 3 - |
| | | | | Homo sapiens |
| NOV77d | CG93669-03 | 1103 | 1104 | NIMA-related protein kinase 3 - |
| | | | | Homo sapiens |
| NOV77e | SNP13376464 | 1105 | 1106 | NIMA-related protein kinase 3 - |
| | | | | Homo sapiens |
| NOV77f | SNP13376462 | 1107 | 1108 | NIMA-related protein kinase 3 - |
| | | | | Homo sapiens |
| NOV77g | SNP13382521 | 1109 | 1110 | NIMA-related protein kinase 3 - |
| | | | | Homo sapiens |
| NOV78a | CG94235-01 | 1111 | 1112 | Thymidylate kinase homologue |
| NOV78b | 254647864 | 1113 | 1114 | Thymidylate kinase homologue |
| NOV78c | 254347797 | 1115 | 1116 | Thymidylate kinase homologue |
| NOV78d | CG94235-02 | 1117 | 1118 | Thymidylate kinase homologue |
| NOV79a | CG95175-01 | 1119 | 1120 | Human EphA full length kinase |
| NOV79b | 275697118 | 1121 | 1122 | Human EphA full length kinase |
| NOV79c | 275697150 | 1123 | 1124 | Human EphA full length kinase |
| NOV80a | CG99638-01 | 1125 | 1126 | Concentrative Na+-nucleoside |
| | | | | cotransporter hCNT3 |
| NOV81a | CG99650-01 | 1127 | 1128 | Concentrative Na+-nucleoside |
| | | | | cotransporter hCNT3 |
| NOV81b | SNP13382525 | 1129 | 1130 | Concentrative Na+-nucleoside |
| | | | | cotransporter hCNT3 |
| NOV81c | SNP13382526 | 1131 | 1132 | Concentrative Na+-nucleoside |
| | | | | cotransporter hCNT3 |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers. SNP analysis for each NOVX, if applicable, is presented in Example D.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 566; (b) a variant of a mature form of the amino acid sequence selected from the group consisting of SEQ ID NO: 2n, wherein n is an integer between 1 and 566, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 566; (d) a variant of the amino acid sequence selected from the group consisting of SEQ ID NO:2n, wherein n is an integer between 1 and 566 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 566; (b) a variant of a mature form of the amino acid sequence selected from the group consisting of SEQ ID NO: 2n, wherein n is an integer between 1 and 566 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 566; (d) a variant of the amino acid sequence selected from the group consisting of SEQ ID NO: 2n, wherein n is an integer between 1 and 566, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 566 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 566; (b) a nucleotide sequence wherein one or more nucleotides in the nucleotide sequence selected from the group consisting of SEQ ID NO: 2n−1, wherein n is an integer between 1 and 566 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 566; and (d) a nucleic acid fragment wherein one or more nucleotides in the nucleotide sequence selected from the group consisting of SEQ ID NO: 2n−1, wherein n is an integer between 1 and 566 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 566, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 566, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 566, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 566, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 566, is one that is sufficiently complementary to the nucleotide sequence of SEQ ID NO:2n−1, wherein n is an integer between 1 and 566, that it can hydrogen bond with few or no mismatches to the nucleotide sequence shown in SEQ ID NO:2n−1, wherein n is an integer between 1 and 566, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g., they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g., Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 566, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g., from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 566; or an anti-sense strand nucleotide sequence of SEQ ID NO:2n−1, wherein n is an integer between 1 and 566; or of a naturally occurring mutant of SEQ ID NO:2n−1, wherein n is an integer between 1 and 566.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 566, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 566, due to degeneracy of the genetic code and thus encode the same NOVX proteins as that encoded by the nucleotide sequences of SEQ ID NO:2n−1, wherein n is an integer between 1 and 566. In another embodiment, an isolated nucleic acid molecule of the invention has a nucleotide sequence encoding a protein having an amino acid sequence of SEQ ID NO:2n, wherein n is an integer between 1 and 566.

In addition to the human NOVX nucleotide sequences of SEQ ID NO:2n−1, wherein n is an integer between 1 and 566, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 566, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 566. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 566, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 566, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 566, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g. Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 566, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 566. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are not particularly amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 566, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 566. Preferably, the protein encoded by the nucleic acid molecule is at least about 60% homologous to SEQ ID NO:2n, wherein n is an integer between 1 and 566; more preferably at least about 70% homologous to SEQ ID NO:2n, wherein n is an integer between 1 and 566; still more preferably at least about 80% homologous to SEQ ID NO:2n, wherein n is an integer between 1 and 566; even more preferably at least about 90% homologous to SEQ ID NO:2n, wherein n is an integer between 1 and 566; and most preferably at least about 95% homologous to SEQ ID NO:2n, wherein n is an integer between 1 and 566.

An isolated nucleic acid molecule encoding a NOVX protein homologous to the protein of SEQ ID NO:2n, wherein n is an integer between 1 and 566, can be created by introducing one or more nucleotide substitutions, additions or deletions into the nucleotide sequence of SEQ ID NO:2n−1, wherein n is an integer between 1 and 566, such that one or more amino acid substitutions, additions or deletions are introduced into the encoded protein.

Mutations can be introduced any one of SEQ ID NO:2n−1, wherein n is an integer between 1 and 566, by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 566, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g., avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ TU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g., inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g., commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×-proteinase-K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in-vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 566, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 566, or antisense nucleic acids complementary to a NOVX nucleic acid sequence of SEQ ID NO:2n−1, wherein n is an integer between 1 and 566, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences that flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N-6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 566). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)-amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 566. The invention also includes a mutant or variant protein any of whose residues may be changed from the corresponding residues shown in any one of SEQ ID NO:2n, wherein n is an integer between 1 and 566, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 566) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 566. In other embodiments, the NOVX protein is substantially homologous to SEQ ID NO:2n, wherein n is an integer between 1 and 566, and retains the functional activity of the protein of SEQ ID NO:2n, wherein n is an integer between 1 and 566, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 566, and retains the functional activity of the NOVX proteins of SEQ ID NO:2n, wherein n is an integer between 1 and 566.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 566.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 566, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g., promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins. (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 566, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen that is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells' is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals. For example, mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g. U.S. Pat. No. 4,946,778). In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand that may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,”. Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 566, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 566), but more preferably, is a non-human homologue of a human NOVX gene. For example, a mouse homologue of human NOVX gene of SEQ ID NO:2n−1, wherein n is an integer between 1 and 566, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds that bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g., a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.) detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g., stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 566, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 566, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 566, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (ise., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265:12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data Example 1

The NOV1 clone was analyzed, and the nucleotide and encoded polypeptide sequences are shown in Table 1A.

TABLE 1A
NOV1 Sequence Analysis

| | | |
|---|---|---|
| NOV1a, CG101340-01 | SEQ ID NO: 1 | 1327 bp |

| | | |
|---|---|---|
| DNA Sequence | ORF Start: ATG at 76 | ORF Stop: TGA at 1192 |

GAGGTGAATGCCATGCCATGATTCTGGTGTGCTCCATGGCATCCCCAGCCTAGCTCCCAATCCCACTT
TGGCACGATGTTAGCCAACAGCTCCTCAACCAACAGTTCTGTTCTCCCGTGTCCTGACTACCGACCTA
CCCACCGCCTGCACTTGGTGGTCTACAGCTTGGTGCTGGCTGCCGGGCTCCCCCTCAACGCGCTAGCC
CTCTGGGTCTTCCTGCGCGCGCTGCGCGTGCACTCGGTGGTGAGCGTGTACATGTGTAACCTGGCGGC
CAGCGACCTGCTCTTCACCCTCTCGCTGCCCGTTCGTCTCTCCTACTACGCACTGCACCACTGGCCCT
TCCCCGACCTCCTGTGCCAGACGACGGGCGCCATCTTCCAGATGAACATGTACGGCAGCTGCATCTTC
CTGATGCTCATCAACGTGGACCGCTACGCCGCCATCGTGCACCCGCTGCGACTGCGCCACCTGTGGCG
GCCCCGCGTGGCGCGGCTGCTCTACCTGGGCGTGTGGGCGCTCATCCTGGTGTTTGCCGTGCCCGCCG
CCCGCGTGCACAGGCCCTCGCGTTGCCGCTACCGGGACCTCGAGGTGCGCCTATGCTTCGAGAGCTTC
TGCGACGAGCTGTGGAAAGGCAGGCTGCTGCCCCTCGTGCTGCTGGCCGAGGCGCTGGGCTTCCTGCT
GCCCCTGGCGGCGGTGGTCTACTCGTCGGGCCGAGTCTTCTGGACGCTGGCGCGCCCCGACGCCACGC
AGAGCCAGCGGCGGTGGAAGACCGTGCGCCTCCTGCTGGCTAACCTCGTCATCTTCCTGCTGTGCTTC
GTGCCCTACAACAGCACGCTGGCGGTCTACGGGCTGCTGCGGAGCAAGCTGGTGGCGGCCAGCGTGCC
TGCCCGCGATCGCGTGCGCGGGGTGCTGATGGTGATGGTGCTGCTGGCCGGCGCCAACTGCGTGCTGG
ACCCGCTGGTGTACTACTTTAGCGCCGAGGGCTTCCGCAACACCCTGCGCGGCCTGGGCACTCCGCAC
CGGGCCAGGACCTCGGCCACCAACGGGACGCGGGCGGCGCTCGCGCAATCCGAAAGGTCCGCCGTCAC
CACCGACGCCACCAGGCCGGATGCCGCCAGTCAGGGGCTGCTCCGACCCTCCGACTCCCACTCTCTGT
CTTCCTTCACACAGTGTCCCCAGGATTCCGCCCTCTGAACACACATGCCATTGCGCTGTCCGTGCCCG
ACTCCCAACGCCTCTCGTTCTGGGAGGCTTACAGGGCGTACACACAAGAAGGTGGGCTGGGCACTTGG
ACCTTTGGGTGGCAATTCCAGCTTAGCAACGCAGA

NOV1a, CG101340-01

| | | | |
|---|---|---|---|
| Protein Sequence | SEQ ID NO: 2 | 372 aa | MW at 41482.1kD |

MLANSSSTNSSVLPCPDYRPTHRLHLVVYSLVLAAGLPLNALALWVFLRALRVHSVVSVYMCNLAASD
LLFTLSLPVRLSYYALHHWPFPDLLCQTTGAIFQMNMYGSCIFLMLINVDRYAAIVHPLRLRHLWRPR
VARLLYLGVWALILVFAVPAARVHRPSRCRYRDLEVRLCFESFCDELWKGRLLPLVLLAEALGFLLPL
AAVVYSSGRVFWTLARPDATQSQRRWKTVRLLLANLVIFLLCFVPYNSTLAVYGLLRSKLVAASVPAR
DRVRGVLMVMVLLAGANCVLDPLVYYFSAEGFRNTLRGLGTPHRARTSATNGTHAALAQSERSAVTTD
ATRPDAASQGLLRPSDSHSLSSFTQCPQDSAL

| | |
|---|---|
| SEQ ID NO: 3 | 1327 bp |

| | | |
|---|---|---|
| NOV1b, SNP13382465 of | ORF Start: ATG at 76 | ORF Stop: TGA at 1192 |

| | | |
|---|---|---|
| CG101340-01, DNA Sequence | SNP Pos: 472 | SNP Change: T to C |

GAGGTGAATGCCATGCCATGATTCTGGTGTGCTCCATGGCATCCCCAGCCTAGCTCCCAATCCCACTT
TGGCACGATGTTAGCCAACAGCTCCTCAACCAACAGTTCTGTTCTCCCGTGTCCTGACTACCGACCTA
CCCACCGCCTGCACTTGGTGGTCTACAGCTTGGTGCTGGCTGCCGGGCTCCCCCTCAACGCGCTAGCC
CTCTGGGTCTTCCTGCGCGCGCTGCGCGTGCAcTCGGTGGTGAGCGTGTACATGTGTAACCTGGCGGC
CAGCGACCTGCTCTTCACCCTCTCGCTGCCCGTTCGTCTCTCCTACTACGCACTGCACCACTGGCCCT
TCCCCGACCTCCTGTGCCAGACGACGGGCGCCATCTTCCAGATGAACATGTACGGCAGCTGCATCTTC
CTGATGCTCATCAACGTGGACCGCTACGCCGCCATCGTGCACCCGCTGCGACTGCGCCACCTGCGGCG
GCCCCGCGTGGCGCGGCTGCTCTACCTGGGCGTGTGGGCGCTCATCCTGGTGTTTGCCGTGCCCGCCG
CCCGCGTGCACAGGCCCTCGCGTTGCCGCTACCGGGACCTCGAGGTGCGCCTATGCTTCGAGAGCTTC
TGCGACGAGCTGTGGAAAGGCAGGCTGCTGCCCCTCGTGCTGCTGGCCGAGGCGCTGGGCTTCCTGCT
GCCCCTGGCGGCGGTGGTCTACTCGTCGGGCCGAGTCTTCTGGACGCTGGCGCGCCCCGACGCCACGC
AGAGCCAGCGGCGGTGGAAGACCGTGCGCCTCCTGCTGGCTAACCTCGTCATCTTCCTGCTGTGCTTC
GTGCCCTACAACAGCACGCTGGCGGTCTACGGGCTGCTGCGGAGCAAGCTGGTGGCGGCCAGCGTGCC
TGCCCGCGATCGCGTGCGCGGGGTGCTGATGGTGATGGTGCTGCTGGCCGGCGCCAACTGCGTGCTGG
ACCCGCTGGTGTACTACTTTAGCGCCGAGGGCTTCCGCAACACCCTGCGCGGCCTGGGCACTCCGCAC
CGGGCCAGGACCTCGGCCACCAACGGGACGCGGGCGGCGCTCGCGCAATCCGAAAGGTCCGCCGTCAC
CACCGACGCCACCAGGCCGGATGCCGCCAGTCAGGGGCTGCTCCGACCCTCCGACTCCCACTCTCTGT
CTTCCTTCACACAGTGTCCCCAGGATTCCGCCCTCTGAACACACATGCCATTGCGCTGTCCGTGCCCG
ACTCCCAACGCCTCTCGTTCTGGGAGGCTTACAGGGCGTACACACAAGAAGGTGGGCTGGGCACTTGG
ACCTTTGGGTGGCAATTCCAGCTTAGCAACGCAGA

| | | |
|---|---|---|
| NOV1b, SNP13382465 of | SEQ ID NO: 4 | MW at 41452.1kD |

| | | | |
|---|---|---|---|
| CG101340-01, Protein Sequence | SNP Pos: 133 | 372 aa | SNP Change: Trp to Arg |

MLANSSSTNSSVLPCPDYRPTHRLHLVVYSLVLAAGLPLNALALWVFLRALRVHSVVSVYMCNLAASD
LLFTLSLPVRLSYYALHHWPFPDLLCQTTGAIFQMNNYGSCIFLMLINVDRYAAIVHPLRLRHLRRPR
VARLLYLGVWALILVFAVPAARVHRPSRCRYRDLEVRLCFESFCDELWKGRLLPLVLLAEALGFLLPL
AAVVYSSGRVFWTLARPDATQSQRRWKTVRLLLANLVIFLLCFVPYNSTLAVYGLLRSKLVAASVPAR
DRVRGVLMVMVLLAGANCVLDPLVYYFSAEGFRNTLRGLGTPHRARTSATNGTRAALAQSERSAVTTD
ATRPDAASQGLLRPSDSHSLSSFTQCPQDSAL

A ClustalW comparison of the above protein sequences yields the following sequence alignment shown in Table 1B.

TABLE 1B
Comparison of the NOV1 protein sequences.

| | |
|---|---|
| NOV18a | MCSLPMARYYRIKYADQKALYTRDGQLLVGDPVADNCCAEKICILPNRGLARTKVPIFLG |
| NOV18b | MCSLPMARYYIIKYADQKALYTRDGQLLVGDPVADNCCAEKICILPNRGLARTKVPIFLG |
| NOV18c | -----LSYCFRIKYADQKALYTRDGQLLVGDPVADNCCAEKICILPNRGLARTKVPIFLG |
| NOV18a | QGGSRCLACVETEEGPSLQLEPSTLPPQDVNIEELYKGGEEATRFTFFQSSSGSAFRLE |
| NOV18b | QGGSRCLACVETEEGPSLQLE-------DVNIEELYKGGEEATRFTFFQSSSGSAFRLE |
| NOV18c | QGGSRCLACVETEEGPSLQLEPSTLPPQDVNIEELYKGGEEATRFTFFQSSSGSAFRLE |
| NOV18a | AAAWPGWFLCGPAEPQQPVQLTKESEPSARTKFYFEQSW |
| NOV18b | AAAWPGWFLCGPAEPQQPVQLTKESEPSARTKFYFEQSW |
| NOV18c | AAAWPGWFLCGPAEPQQPVQLTKESEPSARTKFYFEQSW |
| NOV1a | (SEQ ID NO: 2) |

Further analysis of the NOV1a protein yielded the following properties shown in Table 1C.

TABLE 1C
Protein Sequence Properties NOV1a

| | |
|---|---|
| SignalP analysis: | Cleavage site between residues 59 and 60 |

PSORT II analysis:

| | |
|---|---|
| PSG: | a new signal peptide prediction method |
| | N-region: length 0; pos.chg 0; neg.chg 0 |
| | H-region: length 16; peak value 3.12 |
| | PSG score: −1.28 |
| GvH: | von Heijne's method for signal seq. recognition |
| | GvH score (threshold: −2.1): 0.37 |
| | possible cleavage site: between 43 and 44 |

| | |
|---|---|
| >>> Seems to have no N-terminal signal peptide | |
| ALOM: | Klein et al's method for TM region allocation |
| | Init position for calculation: 1 |
| | Tentative number of TMS(s) for the threshold 0.5: 6 |

| | | |
|---|---|---|
| INTEGRAL | Likelihood = −4.19 | Transmembrane 31-47 |
| INTEGRAL | Likelihood = −0.16 | Transmembrane 99-115 |
| INTEGRAL | Likelihood = −7.01 | Transmembrane 140-156 |
| INTEGRAL | Likelihood = −6.90 | Transmembrane 192-208 |
| INTEGRAL | Likelihood = −7.54 | Transmembrane 232-248 |
| INTEGRAL | Likelihood = −3.82 | Transmembrane 277-293 |

| | |
|---|---|
| PERIPHERAL | Likelihood = 1.43 (at 56) |
| ALOM score: | −7.54 (number of TMSs: 6) |

| | |
|---|---|
| MTOP: | Prediction of membrane topology (Hartmann et al.) |
| | Center position for calculation: 38 |
| | Charge difference: 0.5 C(2.5) − N(2.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment (75): | 4.95 |
| Hyd Moment (95): | 3.49 | G content: | 0 |
| D/E content: | 1 | S/T content: | 6 |
| Score: −3.65 | | | |

```
Gavel: prediction of cleavage sites for mitochondrial preseq
      R-2 motif at 176 CRY|RD
NUCDISC: discrimination of nuclear localization signals
      pat4: none
      pat7: none
      bipartite: none
      content of basic residues: 10.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
      type 1: none
      type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
      Prediction: cytoplasmic
      Reliability: 94.1
COIL: Lupas's algorithm to detect coiled-coil regions
total: 0 residues
--------------------------
Final Results (k = 9/23)
      55.6%: endoplasmic reticulum
      11.1%: Golgi
      11.1%: vacuolar
      11.1%: vesicles of secretory system
      11.1%: mitochondrial
>> prediction for CG101340-01 is end (k = 9)
```

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP81962 | Human G protein-coupled | 1 . . . 372 | 368/372 (98%) | 0.0 |
| | receptor GPR92/3 protein SEQ ID | 1 . . . 372 | 368/372 (98%) | |
| | NO:410 - Homo sapiens, 372 aa. | | | |
| | [WO200261087-A2, 08-AUG- | | | |
| | 2002] | | | |
| AAM52650 | Human G protein-coupled | 1 . . . 372 | 368/372 (98%) | 0.0 |
| | receptor TGR4 - Homo sapiens, | 1 . . . 372 | 368/372 (98%) | |
| | 372 aa. [WO200177326-A1, 18- | | | |
| | OCT-2001] | | | |
| AAU11897 | Human novel G protein-coupled | 1 . . . 372 | 368/372 (98%) | 0.0 |
| | receptor, GPCR9 - Homo sapiens, | 1 . . . 372 | 368/372 (98%) | |
| | 372 aa. [WO200190187-A2, 29- | | | |
| | NOV-2001] | | | |
| AAE16170 | Human G-protein coupled | 1 . . . 372 | 368/372 (98%) | 0.0 |
| | receptor 1 (GCREC-1) protein - | 1 . . . 372 | 368/372 (98%) | |
| | Homo sapiens, 372 aa. | | | |
| | [WO200187937-A2, 22-NOV- | | | |
| | 2001] | | | |
| AAU97915 | Human HIPHUM0000001 | 1 . . . 372 | 368/372 (98%) | 0.0 |
| | purinergic receptor protein - | 1 . . . 372 | 368/372 (98%) | |
| | Homo sapiens, 372 aa. | | | |
| | [GB2360523-A, 26-SEP-2001] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H1C0 | Putative G protein-coupled | 1 . . . 372 | 368/372 (98%) | 0.0 |
| | receptor 92 (Putative G-protein | 1 . . . 372 | 368/372 (98%) | |
| | coupled receptor) - Homo sapiens | | | |
| | (Human), 372 aa. | | | |
| CAD38444 | Sequence 10 from Patent | 3 . . . 372 | 294/370 (79%) | e-164 |
| | WO0238607 - Mus musculus | 123 . . . 488 | 316/370 (84%) | |
| | (Mouse), 488 aa. | | | |
| P32250 | P2Y purinoceptor 5 (P2Y5) | 22 . . . 302 | 111/284 (39%) | 1e-51 |
| | (Purinergic receptor 5) (6H1) - | 14 . . . 293 | 169/284 (59%) | |
| | Gallus gallus (Chicken), 308 aa. | | | |
| Q8BMC0 | P2Y purinoceptor 5 - Mus | 7 . . . 309 | 113/306 (36%) | 1e-49 |
| | musculus (Mouse), 344 aa. | 3 . . . 303 | 178/306 (57%) | |
| P43657 | P2Y purinoceptor 5 (P2Y5) | 22 . . . 309 | 106/291 (36%) | 1e-49 |
| | (Purinergic receptor 5) (RB intron | 17 . . . 303 | 171/291 (58%) | |
| | encoded G-protein coupled | | | |
| | receptor) - Homo sapiens | | | |
| | (Human), 344 aa. | | | |

PFam analysis indicates that the NOV1a protein contains the domains shown in the Table 1F.

TABLE 1F
Domain Analysis of NOV1a

| | | Identities/ | |
|---|---|---|---|
| | | Similarities for | Expect |
| Pfam Domain | NOV1a Match Region | the Matched Region | Value |
| 7tm_1 | 39 . . . 297 | 87/276 (32%) | 7.2e-53 |
| | | 196/276 (71%) | |

### Example 2

The NOV2 clone was analyzed, and the nucleotide and encoded polypeptide sequences are shown in Table 2A.

TABLE 2A
NOV2 Sequence Analysis

| | | |
|---|---|---|
| NOV2a, CG101396-01 | SEQ ID NO: 5 | 3189 bp |

| | | |
|---|---|---|
| DNA Sequence | ORF Start: ATG at 86 | ORF Stop: TGA at 3113 |

CTGCACCGGGACCAGCGCCTCCCCGCTTCGCGCTGCCCTCGGCCTCGCCCCGGGCCCGGGTGGATGAG
CCGCGCGCCCGGGGGACATGGAAGCGCTGACGCTGTGGCTTCTCCCCTGGATATGCCAGTGCGTGTCG
GTGCGGGCCGACTCCATCATCCACATCGGTGCCATCTTCGAGGAGAACGCGGCCAAGGACGACAGGGT
GTTCCAGTTGGCGGTATCCGACCTGAGCCTCAACGATGACATCCTGCAGAGCGAGAAGATCACCTAGT
CCATCAAGGTCATCGAGGCCAACAACCCATTCCAGGCTGTGCAGGAAGCCTGTGACCTCATGACCCAG
GGGATTTTGGCCTTGGTCACGTCCACTGGCTGTGCATCTGCCAATGCCCTGCAGTCCCTCACGGATGC
CATGCACATCCCACACCTCTTTGTCCAGCGCAACCCGGGAGGGTCGCCACGCACCGCATGCCACCTGA
ACCCCAGCCCCGATGGTGAGGCCTACACACTGGCTTCGAGACCACCCGTCCGCCTCAATGATGTCATG
CTCAGGCTGGTGACGGAGCTGCGCTGGCAGAAGTTCGTCATGTTCTACGACAGCGAGTATGATATCCG
TGGGCTTCAAAGCTTTCTGGACCAGGCCTCGCGGCTGGGCCTTGACGTCTCTTTACAAAAGGTGGACA
AGAACATTAGCCACGTATTCACCAGCCTCTTCACCACGATGAAGACAGAGGAGCTGAATCGCTACCGG
GACACGCTTCGCCGCGCCATCCTGCTGCTCAGCCCACAGGGAGCCCACTCCTTCATCAACGAGGCCGT
GGAGACCAACCTGGCTTCCAAGGACAGCCACTGGGTCTTTGTGAATGAGGAAATCAGTGACCCGGAGA
TCCTGGATCTGGTCCATAGTGCCCTTGGAAGGATGACCGTGGTCCGGCAAATCTTTCCGTCTGCAAAG
GACAATCAGAAATGCACGAGGAACAACCACCGCATCTCCTCCCTGCTCTGCGACCCCCAGGAAGGCTA
CCTCCAGATGCTGCAGATCTCCAACCTCTATCTGTATGACAGTGTTCTGATGCTGGCCAACGCCTTTC
ACAGGAAGCTGGAGGACCGGAAGTGGCATAGCATGGCGAGCCTCAACTGCATACGGAAATCCACTAAG
CCATGGAATGGTGGGAGGTCCATGCTGGATACCATCAAAAAGGGCCACATCACTGGCCTCACTGGGGT
GATGGAGTTTCGGGAGGACAGTTCGAATCCCTATGTCCAGTTTGAAATCCTTGGCACTACCTATAGTG
AGACTTTTGGCAAAGACATGCGCAAGTTGGCGACATGGGACTCAGAGAAGGGCTTGAATGGCAGCTTG
CAAGAGAGGCCCATGGGCAGCCGCCTCCAAGGATTGACTCTTAAAGTGGTGACTGTCTTGGAAGAGCC
TTTCGTGATGGTGGCTGAGAACATCCTAGGACAGCCCAAGCGCTACAAAGGGTTCTCCATAGATGTCC
TGGATGCACTGGCCAAGGCTCTGGGCTTTAAATATGAGATTTACCAAGCCCCTGATGGCAGGTACGGT
CACCAGCTCCATAACACCTCCTGGAACGGGATGATCGGGGAGCTCATCAGCAAGAGAGCAGACTTGGC
CATCTCTGCCATCACCATCACCCCAGAGAGGGAGAGCGTTGTGGACTTCAGCAAGCGGTACATGGACT
ATTCAGTGGGGATTCTAATTAAGAAGCCCGAGGAGAAAATCAGCATCTTCTCCCTCTTTGCTCCATTT
GATTTCGCTGTGTGGGCCTGCATTGCAGCAGCCATCCCTGTGGTTGGTGTGCTGATATTTGTGTTGAA
CAGGATACAGGCTGTGAGGGCTCAGAGTGCTGCCCAGCCCAGGCCGTCAGCTTCTGCCACTCTGCACA
GCGCCATCTGGATTGTCTATGGAGCCTTCGTACAGCAAGGTGGCGAATCTTCCGTGAACTCCATGGCC
ATGCGCATCGTGATGGGCAGCTGGTGGCTCTTCACGCTCATTGTGTGCTCCTCCTACACAGCCAACGT
TGCTGCCTTCCTCACAGTGTCCAGGATGGACAACCCCATAAGGACTTTCCAGGACCTGTCCAAACAAG
TGGAAATGTCTTATGGCACTGTCCGGGATTCTGCTGTATATGAGTACTTCCGAGCCAAGGGCACCAAC
CCCCTGGAGCAGGACAGCACGTTTGCTGAACTCTGGCGGACCATCAGCAAGAACGGAGGGGCTGACAA
CTGCGTGTCCAGTCCTTCAGAAGGCATCAGGAAGGCAAAGAAGGGGAACTACGCCTTCCTGTGGGATG
TGGCCGTGGTGGAATACGCAGCCCTGACGGATGACGACTGCTCGGTGACTGTCATCGGCAACAGCATC
AGCAGCAAGGGTTACGGGATTGCCCTGCAGCATGGCAGCCCCTACAGGGACCTCTTCTCCCAGAGGAT
CCTGGAGCTGCAGGACACAGGGGACCTGGATGTGCTGAAGCAGAAGTGGTGGCCGCACATGGGCCGCT
GTGACCTCACCAGCCATGCCAGCGCCCAGGCCGACGGCAAATCCCTCAAGCTGCACAGCTTCGCCGGG
GTCTTCTGCATCCTGGCCATTGGCCTGCTCCTGGCCTGCCTGGTGGCTGCCCTGGAGTTGTGGTGGAA
CAGCAACCGGTGCCACCAGGAGACCCCCAAGGAGGACAAAGAAGTGAACTTGGAGCAGGTCCACCGGC
GCATGAACAGCCTCATGGATGAAGACATTGCTCACAAGCAGATTTCCCCAGCGTCGATTGAGCTCTCG
GCCCTGGAGATGGGGGGCCTGGCTCCCACCCAGACCTTGGAGCCGACACGGGAGTACCAGAACACCCA
GCTCTCGGTCAGCACCTTTCTGCCAGAGCAGAGCAGCCATGGCACCAGCCGGACACTCTCATCAGGGC
CCAGCAGCAACCTGCCGCTGCCGCTGAGCAGCTCGGCGACCATGCCCTCCATGCAGTGCAAACACAGG
TCACCCAACGGGGGGCTGTTCCGGCAGAGCCCGGTGAAGACCCCCATCCCCATGTCCTTCCAGCCCGT
GCCTGGAGGCGTCCTTCCAGAGGCTCTGGACACCTCCCACGGGACCTCCATCTGACTGCGCCGCCTGC
CCTCCTGCCCACCCTCCCACCCACCCGACCAGCAGAGCTTTTTAATACAAGAAAACAACAA

NOV2a, CG101396-01

| | | | |
|---|---|---|---|
| Protein Sequence | SEQ ID NO: 6 | 1009 aa | MW at 112129.7kD |

MEALTLWLLPWICQCVSVRADSIIHIGAIFEENAAKDDRVFQLAVSDLSLNDDILQSEKITYSIKVIE
ANNPFQAVQEACDLMTQGILALVTSTGCASANALQSLTDAMHIPHLFVQRNPGGSPRTACHLNPSPDG
EAYTLASRPPVRLNDVMLRLVTELRWQKFVMFYDSEYDIRGLQSFLDQASRLGLDVSLQKVDKNISHV
FTSLFTTMKTEELNRYRDTLRRAILLLSPQGAHSFINEAVETNLASKDSHWVFVNEEISDPEILDLVH
SALGRMTVVRQIFPSAKDNQKCTRNNHRISSLLCDPQEGYLQMLQISNLYLYDSVLMLANAFHRKLED
RKWHSMASLNCIRKSTKPWNGGRSMLDTIKKGHITGLTGVMEFREDSSNPYVQFEILGTTYSETFGKD
MRKLATWDSEKGLNGSLQERPMGSRLQGLTLKVVTVLEEPFVMVAENILGQPKRYKGFSIDVLDALAK
ALGFKYEIYQAPDGRYGHQLHNTSWNGMIGELISKRADLAISAITITPERESVVDFSKRYMDYSVGIL
IKKPEEKISIFSLFAPFDFAVWACIAAAIPVVGVLIFVLNRIQAVRAQSAAQPRPSASATLHSAIWIV
YGAFVQQGGESSVNSMAMRIVMGSWWLFTLIVCSSYTANLAAFLTVSRMDNPIRTFQDLSKQVEMSYG
TVRDSAVYEYFRAKGTNPLEQDSTFAELWRTISKNGGADNCVSSPSEGIRKAKKGNYAFLWDVAVVEY
AALTDDDCSVTVIGNSISSKGYGIALQHGSPYRDLFSQRILELQDTGDLDVLKQKWWPHMGRCDLTSH
ASAQADGKSLKLHSFAGVFCILAIGLLLACLVAALELWWNSNRCHQETPKEDKEVNLEQVHRRMWSLM
DEDIAHKQISPASIELSALEMGGLAPTQThEPTREYQNTQLSVSTFLPEQSSHGTSRTLSSGPSSNLP
LPLSSSATMPSMQCKHRSPNGGLFRQSPVKTPIPMSFQPVPGGVLPEALDTSHGTSI

| | | |
|---|---|---|
| NOV2b, 267253224 | SEQ ID NO: 7 | 2986 bp |

| | | |
|---|---|---|
| DNA Sequence | ORF Start: at 2 | ORF Stop: end of sequence |

CACCGGTACCGACTCCATCATCCACATCGGTGCCATCTTCGAGGAGAACGCGGCCAAGGACGACAGGG
TGTTCCAGTTGGCGGTATCCGACCTGAGCCTCAACGATGACATCCTGCAGAGCGAGAAGATCACCTAC
TCCATCAAGGTCATCGAGGCCAACAACCCATTCCAGGCTGTGCAGGAAGCCTGTGACCTCATGACCCA
GGGGATTTTGGCCTTGGTCACGTCCACTGGCTGTGCATCTGCCAATGCCCTGCAGTCCCTCACGGATG
CCATGCACATCCCACACCTCTTTGTCCAGCGCAACCCGGGAGGGTCGCCACGCACCGCATGCCACCTG
AACCCCAGCCCCGATGGTGAGGCCTACACACTGGCTTCGAGACCACCCGTCCGCCTCAATGATGTCAT
GCTCAGGCTGGTGACGGAGCTGCGCTGGCAGAAGTTCGTCATGTTCTACGACAGCGAGTATGATATCC
GTGGGCTTCAAAGCTTTCTGGACCAGGCCTCGCGGCTGGGCCTTGACGTCTCTTTACAAAAGGTGGAC
AAGAACATTAGCCACGTATTCACCAGCCTCTTCACCACGATGAAGACAGAGGAGCTGAATCGCTACCG
GGACACGCTTCGCCGCGCCATCCTGCTGCTCAGCCCACAGGGAGCCCACTCCTTCATCAACGAGGCCG
TGGAGACCAACCTGGCTTCCAAGGACAGCCACTGGGTCTTTGTGAATGAGGAAATCAGTGACCCGGAG
ATCCTGGATCTGGTCCATAGTGCCCTTGGAAGGATGACCGTGGTCCGGCAAATCTTTCCGTCTGCAAA
GGACAATCAGAAATGCACGAGGAACAACCACCGCATCTCCTCCCTGCTCTGCGACCCCCAGGAAGGCT
ACCTCCAGATGCTGCAGATCTCCAACCTCTATCTGTATGACAGTGTTCTGATGCTGGCCAACGCCTTT
CACAGGAAGCTGGAGGACCGGAAGTGGCATAGCATGGCGAGCCTCAACTGCATACGGAAATCCACTAA
GCCATGGAATGGTGGGAGGTCCATGCTGGATACCATCAAAAAGGGCCACATCACTGGCCTCACTGGGG
TGATGGAGTTTCGGGAGGACAGTTCGAATCCCTATGTCCAGTTTGAAATCCTTGGCACTACCTATAGT
GAGACTTTTGGCAAAGACATGCGCAAGTTGGCGACATGGGACTCAGAGAAGGGCTTGAATGGCAGCTT
GCAAGAGAGGCCCATGGGCAGCCGCCTCCAAGGATTGACTCTTAAAGTGGTGACTGTCTTGGAAGAGC
CTTTCGTGATGGTGGCTGAGAACATCCTAGGACAGCCCAAGCGCTACAAAGGGTTCTCCATAGATGTC
CTGGATGCACTGGCCAAGGCTCTGGGCTTTAAATATGAGATTTACCAAGCCCCTGATGGCAGGTACGG
TCACCAGCTCCATAACACCTCCTGGAACGGGATGATCGGGGAGCTCATCAGCAAGAGAGCAGACTTGG
CCATCTCTGCCATCACCATCACCCCAGAGAGGGAGAGCGTTGTGGACTTCAGCAAGCGGTACATGGAC
TATTCAGTGGGGATTCTAATTAAGAAGCCCGAGGAGAAAATCAGCATCTTCTCCCTCTTTGCTCCATT
TGATTTCGCTGTGTGGGCCTGCATTGCAGCAGCCATCCCTGTGGTTGGTGTGCTGATATTTGTGTTGA
ACAGGATACAGGCTGTGAGGGCTCAGAGTGCTGCCCAGCCCAGGCCGTCAGCTTCTGCCACTCTGCAC
AGCGCCATCTGGATTGTCTATGGAGCCTTCGTACAGCAAGGTGGCGAATCTTCCGTGAACTCCATGGC
CATGCGCATCGTGATGGGCAGCTGGTGGCTCTTCACGCTCATTGTGTGCTCCTCCTACACAGCCAACC
TTGCTGCCTTCCTCACAGTGTCCAGGATGGACAACCCCATAAGGACTTTCCAGGACCTGTCCAAACAA
GTGGAAATGTCTTATGGCACTGTCCGGGATTCTGCTGTATATGAGTACTTCCGAGCCAAGGGCACCAA
CCCCCTGGAGCAGGACAGCACGTTTGCTGAACTCTGGCGGACCATCAGCAAGAACGGAGGGGCTGACA
ACTGCGTGTCCAGTCCTTCAGAAGGCATCAGGAAGGCAAAGAAGGGGAACTACGCCTTCCTGTGGGAT
GTGGCCGTGGTGGAATACGCAGCCCTGACGGATGACGACTGCTCGGTGACTGTCATCGGCAACAGCAT
CAGCAGCAAGGGTTACGGGATTGCCCTGCAGCATGGCAGCCCCTACAGGGACCTCTTCTCCCAGAGGA
TCCTGGAGCTGCAGGACACAGGGGACCTGGATGTGCTGAAGCAGAAGTGGTGGCCGCACATGGGCCGC
TGTGACCTCACCAGCCATGCCAGCGCCCAGGCCGACGGCAAATCCCTCAAGCTGCACAGCTTCGCCGG
GGTCTTCTGCATCCTGGCCATTGGCCTGCTCCTGGCCTGCCTGGTGGCTGCCCTGGAGTTGTGGTGGA
ACAGCAACCGGTGCCACCAGGAGACCCCCAAGGAGGACAAAGAAGTGAACTTGGAGCAGGTCCACCGG
CGCATGAACAGCCTCATGGATGAAGACATTGCTCACAAGCAGATTTCCCCAGCGTCGATTGAGCTCTC
GGCCCTGGAGATGGGGAGCCTGGCTCCCACCCAGACCTTGGAGCCGACACGGGAGTACCAGAACACCC
AGCTCTCGGTCAGCACCTTTCTGCCAGAGCAGAGCAGCCATGGCACCAGCCGGACACTCTCATCAGGG
CCCAGCAGCAACCTGCCGCTGCCGCTGAGCAGCTCGGCGACCATGCCCTCCATGCAGTGCAAACACAG
GTCACCCAACGGGGGGCTGTTCCGGCAGAGCCCGGTGAAGACCCCCATCCCCATGTCCTTCCAGCCCG
TGCCTGGAGGCGTCCTTCCAGAGGCTCTGGACACCTCCCACGGGACCTCCATCCTCGAGGGC

NOV2b, 267253224

| | | | |
|---|---|---|---|
| Protein Sequence | SEQ ID NO: 8 | 995 aa | MW at 110403.5kD |

TGTDSIIHIGAIFEENAAKDDRVFQLAVSDLSLNDDILQSEKITYSIKVIEANNPFQAVQEACDLMTQ
GILALVTSTGCASANALQSLTDAMHIPHLFVQRNPGGSPRTACHLNPSPDGEAYTLASRPPVRLNDVM
LRLVTELRWQKFVMFYDSEYDIRGLQSFLDQASRLGLDVSLQKVDKNISHVFTSLFTTMKTEELNRYR
DTLRRAILLLSPQGAHSFINEAVETNLASKDSHWVFVNEEISDPEILDLVHSALGRMTVVRQIFPSAK
DNQKCTRNNHRISSLLCDPQEGYLQMLQISNLYLYDSVLMLANAFHRKLEDRKWHSMASLNCIRKSTK
PWNGGRSMLDTIKKGHITGLTGVMEFREDSSNPYVQFEILGTTYSETFGKDMRKLATWDSEKGLNGSL
QERPMGSRLQGLTLKVVTVLEEPFVMVAENILGQPKRYKGFSIDVLDALAKALGFKYEIYQAPDGRYG
HQLHNTSWNGMIGELISKRADLAISAITITPERESVVDFSKRYMDYSVGILIKKPEEKISIFSLFAPF
DFAVWACIAAAIPVVGVLIFVLNRIQAVRAQSAAQPRPSASATLHSAIWIVYGAFVQQGGESSVNSMA
MRIVMGSWWLFTLIVCSSYTANLAAFLTVSRMDNPIRTFQDLSKQVEMSYGTVRDSAVYEYFRAKGTN
PLEQDSTFAELWRTISKNGGADNCVSSPSEGIRKAKKGNYAFLWDVAVVEYAALTDDDCSVTVIGNSI
SSKGYGIALQHGSPYRDLFSQRILELQDTGDLDVLKQKWWPHMGRCDLTSHASAQADGKSLKLHSFAG
VFCILAIGLLLACLVAALELWWNSNRCHQETPKEDKEVNLEQVHRRMNSLMDEDIAHKQISPASIELS
ALEMGSLAPTQTLEPTREYQNTQLSVSTFLPEQSSHGTSRTLSSGPSSNLPLPLSSSATMPSMQCKHR
SPNGGLFRQSPVKTPIPMSFQPVPGGVLPEALDTSHGTSILEG

| | | |
|---|---|---|
| NOV2c, 315490179 | SEQ ID NO: 9 | 3049 bp |

| | | |
|---|---|---|
| DNA Sequence | ORF Start: at 2 | ORF Stop: end of sequence |

CACCGGATCCACCATGGAAGCGCTGACGCTGTGGCTTCTCCCCTGGATATGCCAGTGCGTGTCGGTGC
GGGCCGACTCCATCATCCACATTGGTGCCATCTTCGAGGAGAACGCGGCCAAGGACGACAGGGTGTTC
CAGTTGGCGGTATCCGACCTGAGCCTCAACGATGACATCCTGCAGAGCGAGAAGATCACCTACTCCAT
CAAGGTCATCGAGGCCAACAACCCATTCCAGGCTGTGCAGGAAGCCTGTGACCTCATGACCCAGGGGA
TTTTGGCCTTGGTCACGTCCACTGGCTGTGCATCTGCCAATGCCCTGCAGTCCCTCACGGATGCCATG
CACATCCCACACCTCTTTGTCCAGCGCAACCCGGGAGGGTCGCCACGCACCGCATGCCACCTGAACCC
CAGCCCCGATGGTGAGGCCTACACACTGGCTTCGAGACCACCCGTCCGCCTCAATGATGTCATGCTCA
GGCTGGTGACGGAGCTGCGCTGGCAGAAGTTCGTCATGTTCTACGACAGCGAGTATGATATCCGTGGG
CTTCAAAGCTTTCTGGACCAGGCCTCGCGGCTGGGCCTTGACGTCTCTTTACAAAAGGTGGACAAGAA
CATTAGCCACGTATTCACCAGCCTCTTCACCACGATGAAGACAGAGGAGCTGAATCGCTACCGGGACA
CGCTTCGCCGCGCCATCCTGCTGCTCAGCCCACAGGGAGCCCACTCCTTCATCAACGAGGCCGTGGAG
ACCAACCTGGCTTCCAAGGACAGCCACTGGGTCTTTGTGAATGAGGAAATCAGTGACCCGGAGATCCT
GGATCTGGTCCATAGTGCCCTTGGAAGGATGACCGTGGTCCGGCAAATCTTTCCGTCTGCAAAGGACA
ATCAGAAATGCACGAGGAACAACCACCGCATCTCCTCCCTGCTCTGCGACCCCCAGGAAGGCTACCTC
CAGATGCTGCAGATCTCCAACCTCTATCTGTATGACAGTGTTCTGATGCTGGCCAACGCCTTTCACAG
GAAGCTGGAGGACCGGAAGTGGCATAGCATGGCGAGCCTCAACTGCATACGGAAATCCACTAAGCCAT
GGAATGGTGGGAGGTCCATGCTGGATACCATCAAAAAGGGCCACATCACTGGCCTCACTGGGGTGATG
GAGTTTCGGGAGGACAGTTCGAATCCCTATGTCCAGTTTGAAATCCTTGGCACTACCTATAGTGAGAC
TTTTGGCAAAGACATGCGCAAGTTGGCGACATGGGACTCAGAGAAGGGCTTGAATGGCAGCTTGCAAG
AGAGGCCCATGGGCAGCCGCCTCCAAGGATTGACTCTTAAAGTGGTGACTGTCTTGGAAGAGCCTTTC
GTGATGGTGGCTGAGAACATCCTAGGACAGCCCAAGCGCTACAAAGGGTTCTCCATAGATGTCCTGGA
TGCACTGGCCAAGGCTCTGGGCTTTAAATATGAGATTTACCAAGCCCCTGATGGCAGGTACGGTCACC
AGCTCCATAACACCTCCTGGAACGGGATGATCGGGGAGCTCATCAGCAAGAGAGCAGACTTGGCCATC
TCTGCCATCACCATCACCCCAGAGAGGGAGAGCGTTGTGGACTTCAGCAAGCGGTACATGGACTATTC
AGTGGGGATTCTAATTAAGAAGCCCGAGGAGAAAATCAGCATCTTCTCCCTCTTTGCTCCATTTGATT
TCGCTGTGTGGGCCTGCATTGCAGCAGCCATCCCTGTGGTTGGTGTGCTGATATTTGTGTTGAACAGG
ATACAGGCTGTGAGGGCTCAGAGTGCTGCCCAGCCCAGGCCGTCAGCTTCTGCCACTCTGCACAGCGC
CATCTGGATTGTCTATGGAGCCTTCGTACAGCAAGGTGGCGAATCTTCCGTGAACTCCATGGCCATGC
GCATCGTGATGGGCAGCTGGTGGCTCTTCACGCTCATTGTGTGCTCCTCCTACACAGCCAACCTTGCT
GCCTTCCTCACAGTGTCCAGGATGGACAACCCCATAAGGACTTTCCAGGACCTGTCCAAACAAGTGGA
AATGTCTTATGGCACTGTCCGGGATTCTGCTGTATATGAGTACTTCCGAGCCAAGGGCACCAACCCCC
TGGAGCAGGACAGCACGTTTGCTGAACTCTGGCGGACCATCAGCAAGAACGGAGGGGCTGACAACTGC
GTGTCCAGTCCTTCAGAAGOCATCAGGAAGGCAAAGAAGGGGAACTACGCCTTCCTGTGGGATGTGGC
CGTGGTGGAATACGCAGCCCTGACGGATGACGACTGCTCGGTGACTGTCATCGGCAACAGCATCAGCA
GCAAGGGTTACGGGATTGCCCTGCAGCATGGCAGCCCCTACAGGGACCTCTTCTCCCAGAGGATCCTG
GAGCTGCAGGACACAGGGGACCTGGATGTGCTGAAGCAGAAGTGGTGGCCGCACATGGGCCGCTGTGA
CCTCACCAGCCATGCCAGCGCCCAGGCCGACGGCAAATCCCTCAAGCTGCACAGCTTCGCCGGGGTCT
TCTGCATCCTGGCCATTGGCCTGCTCCTGGCCTGCCTGGTGGCTGCCCTGGAGTTGTGGTGGAACAGC
AACCGGTGCCACCAGGAGACCCCCAAGGAGGACAAAGAAGTGAACTTGGAGCAGGTCCACCGGCGCAT
GAACAGCCTCATGGATGAAGACATTGCTCACAAGCAGATTTCCCCAGCGTCGATTGAGCTCTCGGCCC
TGGAGATGGGGGGCCTGGCTCCCACCCAGACCTTGGAGCCGACACGGGAGTACCAGAACACCCAGCTC
TCGGTCAGCACCTTTCTGCCAGAGCAGAGCAGCCATGGCACCAGCCGGACACTCTCATCAGGGCCCAG
CAGCAACCTGCCGCTGCCGCTGAGCAGCTCGGCGACCATGCCCTCCATGCAGTGCAAACACAGGTCAC
CCAACGGGGGGCTGTTCCGGCAGAGCCCGGTGAAGACCCCCATCCCCATGTCCTTCCAGCCCGTGCCT
GGAGGCGTCCTTCCAGAGGCTCTGGACACCTCCCACGGGACCTCCATCCTCGAGGGC

NOV2c, 315490179

| | | | |
|---|---|---|---|
| Protein Sequence | SEQ ID NO: 10 | 1016 aa | MW at 112775.3kD |

TGSTMEALTLWLLPWICQCVSVRADSIIHIGAIFEENAAKDDRVFQLAVSDLSLNDDILQSEKITYSI
KVIEANNPFQAVQEACDLMTQGILALVTSTGCASANALQSLTDAMHIPHLFVQRNPGGSPRTACHLNP
SPDGEAYTLASRPPVRLNDVMLRLVTELRWQKFVMFYDSEYDIRGLQSFLDQASRLGLDVSLQKVDKN
ISHVFTSLFTTMKTEELNRYRDTLRRAILLLSPQGAHSFINEAVETNLASKDSHWVFVNEEISDPEIL
DLVHSALGRMTVVRQIFPSAKDNQKCTRNNHRISSLLCDPQEGYLQMLQISNLYLYDSVLMLANAFHR
KLEDRKWHSMASLNCIRKSTKPWNGGRSMLDTIKKGHITGLTGVMEFREDSSNPYVQFEILGTTYSET
FGKDMRKLATWDSEKGLNGSLQERPMGSRLQGLTLKVVTVLEEPFVMVAENILGQPKRYKGFSIDVLD
ALAKALGFKYEIYQAPDGRYGHQLHNTSWNGMIGELISKRADLAISAITITPERESVVDFSKRYMDYS
VGILIKKPEEKISIFSLFAPFDFAVWACIAAAIPVVGVLIFVLNRIQAVRAQSAAQPRPSASATLHSA
IWIVYGAFVQQGGESSVNSMAMRIVMGSWWLFTLIVCSSYTANLAAFLTVSRMDNPIRTFQDLSKQVE
MSYGTVRDSAVYEYFRAKGTNPLEQDSTFAELWRTISKNGGADNCVSSPSEGIRKAKKGNYAFLWDVA
VVEYAALTDDDCSVTVIGNSISSKGYGIALQHGSPYRDLFSQRILELQDTGDLDVLKQKWWPHMGRCD
LTSHASAQADGKSLKLHSFAGVFCILAIGLLLACLVAALELWWNSNRCHQETPKEDKEVNLEQVHRRM
NSLMDEDIAHKQISPASIELSALEMGGLAPTQTLEPTREYQNTQLSVSTFLPEQSSHGTSRTLSSGPS
SNLPLPLSSSATMPSMQCKHRSPNGGLFRQSPVKTPIPMSFQPVPGGVLPEALDTSHGTSILEG

| | | |
|---|---|---|
| NOV2d, CG101396-02 | SEQ ID NO: 11 | 3094bp |

| | | |
|---|---|---|
| DNA Sequence | ORF Start: ATG at 56 | ORF Stop: TAG at 3083 |

TGTCGACGGCGCCAGTGTGATGATATTGCAGATTCGCCTTCACCGCGGCCGCACCATGGAAGCGCTGA
CGCTGTGGCTTCTCCCCTGGATATGCCAGTGCGTGTCGGTGCGGGCCGACTCCATCATCCACATCGGT
GCCATCTTCGAGGAGAACGCGGCCAAGGACGACAGGGTGTTCCAGTTGGCGGTATCCGACCTGAGCCT
CAGCGATGACATCCTGCAGAGCGAGAAGATCACCTACTCCATCAAGGTCATCGAGGCCAACAACCCAT
TCCAGGCTGTGCAGGAAGCCTGTGACCTCATGACCCAGGGGATTTTGGCCTTGGTCACGTCCACTGGC
TGTGCATCTGCCAATGCCCTGCAGTCCCTCACGGATGCCATGCACATCCCACACCTCTTTGTCCAGCG
CAACCCGGGAGGGTCGCCACGCACCGCATGCCACCTGAACCCCAGCCCCGATGGTGAGGCCTACACAC
TGGCTTCGAGACCACCCGTCCGCCTCAATGATGTCATGCTCAGGCTGGTGACGGAGCTGCGCTGGCAG
AAGTTCGTCATGTTCTACGACAGCGAGTATGATATCCGTGGGCTTCAAAGCTTTCTGGACCAGGCCTC
GCGGCTGGGCCTTGACGTCTCTTTACAAAAGGTGGACAAGAACATTAGCCACGTATTCACCAGCCTCT
TCACCACGATGAAGACAGAGGAGCTGAATCGCTACCGGGACACGCTTCGCCGCGCCATCCTGCTGCTC
AGCCCACAGGGAGCCCACTCCTTCATCAACGAGGCCGTGGAGACCAACCTGGCTTCCAAGGACAGCCA
CTGGGTCTTTGTGAATGAGGAAATCAGTGACCCGGAGATCCTGGATCTGGTCCATAGTGCCCTTGGAA
GGATGACCGTGGTCCGGCAAATCTTTCCGTCTGCAAAGGACAATCAGAAATGCACGAGGAACAACCAC
CGCATCTCCTCCCTGCTCTGCGACCCCCAGGAAGGCTACCTCCAGATGCTGCAGATCTCCAACCTCTA
TCTGTATGACAGTGTTCTGATGCTGGCCAACGCCTTTCACAGGAAGCTGGAGGACCGGAAGTGGCATA
GCATGGCGAGCCTCAACTGCATACGGAAATCCACTAAGCCATGGAATGGTGGGAGGTCCATGCTGGAT
ACCATCAAAAAGGGCCACATCACTGGCCTCACTGGGGTGATGGAGTTTCGGGAGGACAGTTCGAATCC
CTATGTCCAGTTTGAAATCCTTGGCACTACCTATAGTGAGACTTTTGGCAAAGACATGCGCAAGTTGG
CGACATGGGACTCAGAGAAGGGCTTGAATGGCAGCTTGCAAGAGAGGCCCATGGGCAGCCGCCTCCAA
GGATTGACTCTTAAAGTGGTGACTGTCTTGGAAGAGCCTTTCGTGATGGTGGCTGAGAACATCCTAGG
ACAGCCCAAGCGCTACAAAGGGTTCTCCATAGATGTCCTGGATGCACTGGCCAAGGCTCTGGGCTTTA
AATATGAGATTTACCAAGCCCCTGATGGCAGGTACGGTCACCAGCTCCATAACACCTCCTGGAACGGG
ATGATCGGGGAGCTCATCAGCAAGAGAGCAGACTTGGCCATCTCTGCCATCACCATCACCCCAGAGAG
GGAGAGCGTTGTGGACTTCAGCAAGCGGTACATGGACTATTCAGTGGGGATTCTAATTAAGAAGCCCG
AGGAGAAAATCAGCATCTTCTCCCTCTTTGCTCCATTTGATTTCGCTGTGTGGGCCTGCATTGCAGCA
GCCATCCCTGTGGTTGGTGTGCTGATATTTGTGTTGAACAGGATACAGGCTGTGAGGGCTCAGAGTGC
TGCCCAGCCCAGGCCGTCAGCTTCTGCCACTCTGCACAGCGCCATCTGGATTGTCTATGGAGCCTTCG
TACAGCAAGGTGGCGAATCTTCCGTGAACTCCATGGCCATGCGCATCGTGATGGGCAGCTGGTGGCTC
TTCACGCTCATTGTGTGCTCCTCCTACACAGCCAACCTTGCTGCCTTCCTCACAGTGTCCAGGATGGA
CAACCCCATAAGGACTTTCCAGGACCTGTCCAAACAAGTGGAAATGTCTTATGGCACTGTCCGGGATT
CTGCTGTATATGAGTACTTCCGAGCCAAGGGCACCAACCCCCTGGAGCAGGACAGCACGTTTGCTGAA
CTCTGGCGGACCATCAGCAAGAACGGAGGGGCTGACAACTGCGTGTCCAGTCCTTCAGAAGGCATCAG
GAAGGCAAAGAAGGGGAACTACGCCTTCCTGTGGGATGTGGCCGTGGTGGAATACGCAGCCCTGACGG
ATGACGACTGCTCGGTGACTGTCATCGGCAACAGCATCAGCAGCAAGGGTTACGGGATTGCCCTGCAG
CATGGCAGCCCCTACAGGGACCTCTTCTCCCAGAGGATCCTGGAGCTGCAGGACACAGGGGACCTGGA
TGTGCTGAAGCAGAAGTGGTGGCCGCACATGGGCCGCTGTGACCTCACCAGCCATGCCAGCGCCCAGG
CCGACGGCAAATCCCTCAAGCTGCACAGCTTCGCCGGGGTCTTCTGCATCCTGGCCATTGGCCTGCTC
CTGGCCTGCCTGGTGGCTGCCCTGGAGTTGTGGTGGAACAGCAACCGGTGCCACCAGGAGACCCCCAA
GGAGGACAAAGAAGTGAACTTGGAGCAGGTCCACCGGCGCATGAACAGCCTCATGGATGAAGACATTG
CTCACAAGCAGATTTCCCCAGCGTCGATTGAGCTCTCGGCCCTGGAGATGGGGGGCCTGGCTCCCACC
CAGACCTTGGAGCCGACACGGGAGTACCAGAACACCCAGCTCTCGGTCAGCACCTTTCTGCCAGAGCA
GAGCAGCCATGGCACCAGCCGGACACTCTCATCAGGGCCCAGCAGCAACCTGCCGCTGCCGCTGAGCA
GCTCGGCGACCATGCCCTCCATGCAGTGCAAACACAGGTCACCCAACGGGGGGCTGTTCCGGCAGAGC
CCGGTGAAGACCCCCATCCCCATGTCCTTCCAGCCCGTGCCTGGAGGCGTCCTTCCAGAGGCTCTGGA
CACCTCCCACGGGACCTCCATCTAGCTCGAGGGC

NOV2d, CG101396-02

| | | | |
|---|---|---|---|
| Protein Sequence | SEQ ID NO: 12 | 1009 aa | MW at 112102.6kD |

MEALTLWLLPWICQCVSVRADSIIHIGAIFEENAAKDDRVFQLAVSDLSLSDDILQSEKITYSIKVIE
ANNPFQAVQEACDLMTQGILALVTSTGCASANALQSLTDANHIPHLFVQRNPGGSPRTACHLNPSPDG
EAYTLASRPPVRLNDVMLRLVTELRWQKFVMFYDSEYDIRGLQSFLDQASRLGLDVSLQKVDKNISHV
FTSLFTTMKTEELNRYRDTLRRAILLLSPQGAHSFINEAVETNLASKDSHWVFVNEEISDPEILDLVH
SALGRMTVVRQIFPSAKDNQKCTRNNHRISSLLCDPQEGYLQMLQISNLYLYDSVLMLANAFHRKLED
RKWHSMASLNCIRKSTKPWNGGRSMLDTIKKGHITGLTGVMEFREDSSNPYVQFEILGTTYSETFGKD
MRKLATWDSEKGLMGSLQERPMGSRLOGLTLKVVTVLEEPFVMVAENILGQPKRYKGFSIDVLDALAK
ALGFKYEIYQAPDGRYGHQLHNTSWNGMIGELISKRADLAISAITITPERESVVDFSKRYMDYSVGIL
IKKPEEKISIFSLFAPFDFAVWACIAAAIPVVGVLIFVLNRIQAVRAQSAAQPRPSASATLHSAIWIV
YGAFVQQGGESSVNSMAMRIVMGSWWLFTLIVCSSYTANLAAFLTVSRMDNPIRTFQDLSKQVEMSYG
TVRDSAVYEYFRAKGTNPLEQDSTFAELWRTISKNGGADNCVSSPSEGIRKAKKGNYAFLWDVAVVEY
AALTDDDCSVTVIGNSISSKGYGIALQHGSPYRDLFSQRILELQDTGDLDVLKQKWWPHMGRCDLTSH
ASAQADGKSLKLHSFAGVFCILAIGLLLACLVAALELWWNSNRCHQETPKEDKEVNLEQVHRRMNSLM
DEDIAHKQISPASIELSALEMGGLAPTQTLEPTREYQNTQLSVSTFLPEQSSHGTSRTLSSGPSSNLP
LPLSSSATMPSMQCKHRSPNGGLFRQSPVKTPIPMSFQPVPGGVLPEALDTSHGTSI

| | |
|---|---|
| SEQ ID NO: 13 | 3189 bp |

| | | |
|---|---|---|
| NOV2e, SNP13379211 of | ORF Start: ATG at 86 | ORF Stop: TGA at 3113 |

| | | |
|---|---|---|
| CG101396-01, DNA Sequence | SNP Pos: 3184 | SNP Change: C to T |

CTGCACCGGGACCAGCGCCTCCCCGCTTCGCGCTGCCCTCGGCCTCGCCCCGGGCCCGGGTGGATGAG
CCGCGCGCCCGGGGGACATGGAAGCGCTGACGCTGTGGCTTCTCCCCTGGATATGCCAGTGCGTGTCG
GTGCGGGCCGACTCCATCATCCACATCGGTGCCATCTTCGAGGAGAACGCGGCCAAGGACGACAGGGT
GTTCCAGTTGGCGGTATCCGACCTGAGCCTCAACGATGACATCCTGCAGAGCGAGAAGATCACCTACT
CCATCAAGGTCATCGAGGCCAACAACCCATTCCAGGCTGTGCAGGAAGCCTGTGACCTCATGACCCAG
GGGATTTTGGCCTTGGTCACGTCCACTGGCTGTGCATCTGCCAATGCCCTGCAGTCCCTCACGGATGC
CATGCACATCCCGCACCTCTTTGTCCAGCGCAACCCGGGAGGGTCGCCACGCACCGCATGCCACCTGA
ACCCCAGCCCCGATGGTGAGGCCTACACACTGGCTTCGAGACCACCCGTCCGCCTCAATGATGTCATG
CTCAGGCTGGTGACGGAGCTGCGCTGGCAGAAGTTCGTCATGTTCTACGACAGCGAGTATGATATCCG
TGGGCTTCAAAGCTTTCTGGACCAGGCCTCGCGGCTGGGCCTTGACGTCTCTTTACAAAAGGTGGACA
AGAACATTAGCCACGTATTCACCAGCCTCTTCACCACGATGAAGACAGAGGAGCTGAATCGCTACCGG
GACACGCTTCGCCGCGCCATCCTGCTGCTCAGCCCACAGGGAGCCCACTCCTTCATCAACGAGGCCGT
GGAGACCAACCTGGCTTCCAAGGACAGCCACTGGGTCTTTGTGAATGAGGAAATCAGTGACCCGGAGA
TCCTGGATCTGGTCCATAGTGCCCTTGGAAGGATGACCGTGGTCCGGCAAATCTTTCCGTCTGCAAAG
GACAATCAGAAATGCACGAGGAACAACCACCGCATCTCCTCCCTGCTCTGCGACCCCCAGGAAGGCTA
CCTCCAGATGCTGCAGATCTCCAACCTCTATCTGTATGACAGTGTTCTGATGCTGGCCAACGCCTTTC
ACAGGAAGCTGGAGGACCGGAAGTGGCATAGCATGGCGAGCCTCAACTGCATACGGAAATCCACTAAG
CCATGGAATGGTGGGAGGTCCATGCTGGATACCATCAAAAAGGGCCACATCACTGGCCTCACTGGGGT
GATGGAGTTTCGGGAGGACAGTTCGAATCCCTATGTCCAGTTTGAAATCCTTGGCACTACCTATAGTG
AGACTTTTGGCAAAGACATGCGCAAGTTGGCGACATGGGACTCAGAGAAGGGCTTGAATGGCAGCTTG
CAAGAGAGGCCCATGGGCAGCCGCCTCCAAGGATTGACTCTTAAAGTGGTGACTGTCTTGGAAGAGCC
TTTCGTGATGGTGGCTGAGAACATCCTAGGACAGCCCAAGCGCTACAAAGGGTTCTCCATAGATGTCC
TGGATGCACTGGCCAAGGCTCTGGGCTTTAAATATGAGATTTACCAAGCCCCTGATGGCAGGTACGGT
CACCAGCTCCATAACACCTCCTGGAACGGGATGATCGGGGAGCTCATCAGCAAGAGAGCAGACTTGGC
CATCTCTGCCATCACCATCACCCCAGAGAGGGAGAGCGTTGTGGACTTCAGCAAGCGGTACATGGACT
ATTCAGTGGGGATTCTAATTAAGAAGCCCGAGGAGAAAATCAGCATCTTCTCCCTCTTTGCTCCATTT
GATTTCGCTGTGTGGGCCTGCATTGCAGCAGCCATCCCTGTGGTTGGTGTGCTGATATTTGTGTTGAA
CAGGATACAGGCTGTGAGGGCTCAGAGTGCTGCCCAGCCCAGGCCGTCAGCTTCTGCCACTCTGCACA
GCGCCATCTGGATTGTCTATGGAGCCTTCGTACAGCAAGGTGGCGAATCTTCCGTGAACTCCATGGCC
ATGCGCATCGTGATGGGCAGCTGGTGGCTCTTCACGCTCATTGTGTGCTCCTCCTACACAGCCAACCT
TGCTGCCTTCCTCACAGTGTCCAGGATGGACAACCCCATAAGGACTTTCCAGGACCTGTCCAAACAAG
TGGAAATGTCTTATGGCACTGTCCGGGATTCTGCTGTATATGAGTACTTCCGAGCCAAGGGCACCAAC
CCCCTGGAGCAGGACAGCACGTTTGCTGAACTCTGGCGGACCATCAGCAAGAACGGAGGGGCTGACAA
CTGCGTGTCCAGTCCTTCAGAAGGCATCAGGAAGGCAAAGAAGGGGAACTACGCCTTCCTGTGGGATG
TGGCCGTGGTGGAATACGCAGCCCTGACGGATGACGACTGCTCGGTGACTGTCATCGGCAACAGCATC
AGCAGCAAGGGTTACGGGATTGCCCTGCAGCATGGCAGCCCCTACAGGGACCTCTTCTCCCAGAGGAT
CCTGGAGCTGCAGGACACAGGGGACCTGGATGTGCTGAAGCAGAAGTGGTGGCCGCACATGGGCCGCT
GTGACCTCACCAGCCATGCCAGCGCCCAGGCCGACGGCAAATCCCTCAAGCTGCACAGCTTCGCCGGG
GTCTTCTGCATCCTGGCCATTGGCCTGCTCCTGGCCTGCCTGGTGGCTGCCCTGGAGTTGTGGTGGAA
CAGCAACCGGTGCCACCAGGAGACCCCCAAGGAGGACAAAGAAGTGAACTTGGAGCAGGTCCACCGGC
GCATGAACAGCCTCATGGATGAAGACATTGCTCACAAGCAGATTTCCCCAGCGTCGATTGAGCTCTCG
GCCCTGGAGATGGGGGGCCTGGCTCCCACCCAGACCTTGGAGCCGACACGGGAGTACCAGAACACCCA
GCTOTCGGTCAGCACCTTTCTGCCAGAGCAGAGCAGCCATGGCACCAGCCGGACACTCTCATCAGGGC
CCAGCAGCAACCTGCCGCTGCCGCTGAGCAGCTCGGCGACCATGCCCTCCATGCAGTGCAAACACAGG
TCACCCAACGGGGGGCTGTTCCGGCAGAGCCCGGTGAAGACCCCCATCCCCATGTCCTTCCAGCCCGT
GCCTGGAGGCGTCCTTCCAGAGGCTCTGGACACCTCCCACGGGACCTCCATCTGACTGCGCCGCCTGC
CCTCCTGCCCACCCTCCCACCCACCCGACCAGCAGAGCTTTTTAATACAAGAAAATAACAA

| | |
|---|---|
| NOV2e, SNP13379211 of | MW at 112129.7kD |

| | | | |
|---|---|---|---|
| CG101396-01, Protein Sequences | SEQ ID NO: 14 | 1009 aa | SNP Change: no change |

MEALTLWLLPWICQCVSVRADSIIHIGAIFEENAAKDDRVFQLAVSDLSLNDDILQSEKITYSIKVIE
ANNPFQAVQEACDLMTQGILALVTSTGCASANALQSLTDAMHIPHLFVQRNPGGSPRTACHLNPSPDG
EAYTLASRPPVRLNDVMLRLVTELRWQKFVMFYDSEYDIRGLQSFLDQASRLGLDVSLQKVDKNISHV
FTSLFTTMKTEELNRYRDTLRRAILLLSPQGAHSFINEAVETNLASKDSHWVFVNEEISDPEILDLVH
SALGRMTVVRQIFPSAKDNQKCTRNNHRISSLLCDPQEGYLQMLQISNLYLYDSVLMLANAFHRKLED
RKWHSMASLNCIRKSTKPWNGGRSMLDTIKKGHITGLTGVMEFREDSSNPYVQFEILGTTYSETFGKD
MRKLATWDSEKGLNGSLQERPMGSRLQGLTLKVVTVLEEPFVMVAENILGQPKRYKGFSIDVLDALAK
ALGFKYEIYQAPDGRYGHQLHNTSWNGMIGELISKRADLAISAITITPERESVVDFSKRYMDYSVGIL
IKKPEEKISIFSLFAPFDFAVWACIAAAIPVVGVLIFVLNRIQAVRAQSAAQPRPSASATLHSAIWIV
YGAFVQQGGESSVNSMAMRIVMGSWWLFTLIVCSSYTANLAAFLTVSRMDNPIRTFQDLSKQVEMSYG
TVRDSAVYEYFRAKGTNPLEQDSTFAELWRTISKNGGADNCVSSPSEGIRKAKKGNYAFLWDVAVVEY
AALTDDDCSVTVIGNSISSKGYGIALQHGSPYRDLFSQRILELQDTGDLDVLKQKWWPHMGRCDLTSH
ASAQADGKSLKLHSFAGVFCILAIGLLLACLVAALELWWNSNRCHQETPKEDKEVNLEQVHRRMNSLM
DEDIAHKQISPASIELSALEMGGLAPTQTLEPTREYQNTQLSVSTFLPEQSSHGTSRTLSSGPSSNLP
LPLSSSATMPSMQCKHRSPNGGLFRQSPVKTPIPMSFQPVPGGVLPEALDTSHGTSI

A ClustalW comparison of the above protein sequences yields the following sequence alignment shown in Table 2B.

TABLE 2B
Comparison of the NOV2 protein sequences.

| | |
|---|---|
| NOV2a | ----MEALTLWLLPWICQCVSVRASIIHIGAIFEENAAKDDRVFQLAVSDLSLNDDILQ |
| NOV2b | ---------------------TGTDSIIHIGAIFEENAAKDDRVFQLAVSDLSNDDILQ |
| NOV2c | TGSTMEALTLWLLPWICQCVSVRADSIIHIGAIFEENAAKDDRVFQLAVSDLSLNDDILQ |
| NOV2d | ----MEALTLWLLPWICQCVSVRADSIIHIGAIFEENAAKDDRVFQLAVSDLSLNDDILQ |
| NOV2a | SEKITYSIKVIEANNPFQAVQEACDLMTQGILALVTSTGCASANALQSLTDAMHIPHLFV |
| NOV2b | SEKITYSIKVIEANNPFQAVQEACDLMTQGILALVTSTGCASANALQSLTDAMHIPHLFV |
| NOV2c | SEKITYSIKVIEANNPFQAVQEACDLMTQGILALVTSTGCASANALQSLTDAMHIPHLFV |
| NOV2d | SEKITYSIKVIEANNPFQAVQEACDLMTQGILALVTSTGCASANALQSLTDAMHIPHLFV |
| NOV2a | QRNPGGSPRTACHLNPSPDGEAYTLASRPPVRLNDVMLRLVTELRWQKFVMFYDSEYDIR |
| NOV2b | QRNPGGSPRTACHLNPSPDGEAYTLASRPPVRLNDVMLRLVTELRWQKFVMFYDSEYDIR |
| NOV2c | QRNPGGSPRTACHLNPSPDGEAYTLASRPPVRLNDVMLRLVTELRWQKFVMFYDSEYDIR |
| NOV2d | QRNPGGSPRTACHLNPSPDGEAYTLASRPPVRLNDVMLRLVTELRWQKFVMFYDSEYDIR |
| NOV2a | GLQSFLDQASRLGLDVSLQKVDKNISHVFTSLFTTMKTEELNRYRDTLRRAILLLSPQGA |
| NOV2b | GLQSFLDQASRLGLDVSLQKVDKNISHVFTSLFTTMKTEELNRYRDTLRRAILLLSPQGA |
| NOV2c | GLQSFLDQASRLGLDVSLQKVDKNISHVFTSLFTTMKTEELNRYRDTLRRAILLLSPQGA |
| NOV2d | GLQSFLDQASRLGLDVSLQKVDKNISHVFTSLFTTMKTEELNRYRDTLRRAILLLSPQGA |
| NOV2a | HSFINEAVETNLASKDSHWVFVNEEESDPEILDLVHSALGRMTVVRQIFPSAKDNQKCTR |
| NOV2b | HSFINEAVETNLASKDSHWVFVNEEESDPEILDLVHSALGRMTVVRQIFPSAKDNQKCTR |
| NOV2c | HSFINEAVETNLASKDSHWVFVNEEESDPEILDLVHSALGRMTVVRQIFPSAKDNQKCTR |
| NOV2d | HSFINEAVETNLASKDSHWVFVNEEESDPEILDLVHSALGRMTVVRQIFPSAKDNQKCTR |
| NOV2a | NNHRISSLLCDPQEGYLQMLQISNLYLYDSVLMLANAFHRKLEDRKWHSMASLNCIRKST |
| NOV2b | NNHRISSLLCDPQEGYLQMLQISNLYLYDSVLMLANAFHRKLEDRKWHSMASLNCIRKST |
| NOV2c | NNHRISSLLCDPQEGYLQMLQISNLYLYDSVLMLANAFHRKLEDRKWHSMASLNCIRKST |
| NOV2d | NNHRISSLLCDPQEGYLQMLQISNLYLYDSVLMLANAFHRKLEDRKWHSMASLNCIRKST |
| NOV2a | KPWNGGRSMLDTIKKGHITGLTGVMEFREDSSNPYVQFEILGTTYSETFGKDMRKLATWD |
| NOV2b | KPWNGGRSMLDTIKKGHITGLTGVMEFREDSSNPYVQFEILGTTYSETFGKDMRKLATWD |
| NOV2c | KPWNGGRSMLDTIKKGHITGLTGVMEFREDSSNPYVQFEILGTTYSETFGKDMRKLATWD |
| NOV2d | KPWNGGRSMLDTIKKGHITGLTGVMEFREDSSNPYVQFEILGTTYSETFGKDMRKLATWD |
| NOV2a | SEKGLNGSLQERPMGSRLQGLTLKVVTVLEEPFVMVAENILGQPKRYKGFSIDVLDALAK |
| NOV2b | SEKGLNGSLQERPMGSRLQGLTLKVVTVLEEPFVMVAENILGQPKRYKGFSIDVLDALAK |
| NOV2c | SEKGLNGSLQERPMGSRLQGLTLKVVTVLEEPFVMVAENILGQPKRYKGFSIDVLDALAK |
| NOV2d | SEKGLNGSLQERPMGSRLQGLTLKVVTVLEEPFVMVAENILGQPKRYKGFSIDVLDALAK |
| NOV2a | ALGFKYEIYQAPDGRYGHQLHNTSWNGMIGELISKRADLAISAITITPERESVVDFSKRY |
| NOV2b | ALGFKYEIYQAPDGRYGHQLHNTSWNGMIGELISKRADLAISAITITPERESVVDFSKRY |
| NOV2c | ALGFKYEIYQAPDGRYGHQLHNTSWNGMIGELISKRADLAISAITITPERESVVDFSKRY |
| NOV2d | ALGFKYEIYQAPDGRYGHQLHNTSWNGMIGELISKRADLAISAITITPERESVVDFSKRY |
| NOV2a | MDYSVGILIKKPEEKISIFSLFAPFDFAVWACIAAAIPVVGVLIFVLNRIQAVRAQSAAQ |
| NOV2b | MDYSVGILIKKPEEKISIFSLFAPFDFAVWACIAAAIPVVGVLIFVLNRIQAVRAQSAAQ |
| NOV2c | MDYSVGILIKKPEEKISIFSLFAPFDFAVWACIAAAIPVVGVLIFVLNRIQAVRAQSAAQ |
| NOV2d | MDYSVGILIKKPEEKISIFSLFAPFDFAVWACIAAAIPVVGVLIFVLNRIQAVRAQSAAQ |
| NOV2a | PRPSASATLHSAIWIVYGAFVQQGGESSVNSMAMRIVMGSWWLFTLIVCSSYTANLAAFL |
| NOV2b | PRPSASATLHSAIWIVYGAFVQQGGESSVNSMAMRIVMGSWWLFTLIVCSSYTANLAAFL |
| NOV2c | PRPSASATLHSAIWIVYGAFVQQGGESSVNSMAMRIVMGSWWLFTLIVCSSYTANLAAFL |
| NOV2d | PRPSASATLHSAIWIVYGAFVQQGGESSVNSMAMRIVMGSWWLFTLIVCSSYTANLAAFL |
| NOV2a | TVSRMDNPIRTFQDLSKQVEMSYGTVRDSAVYEYFRAKGTNPLEQDSTFAELWRTISKNG |
| NOV2b | TVSRMDNPIRTFQDLSKQVEMSYGTVRDSAVYEYFRAKGTNPLEQDSTFAELWRTISKNG |
| NOV2c | TVSRMDNPIRTFQDLSKQVEMSYGTVRDSAVYEYFRAKGTNPLEQDSTFAELWRTISKNG |
| NOV2d | TVSRMDNPIRTFQDLSKQVEMSYGTVRDSAVYEYFRAKGTNPLEQDSTFAELWRTISKNG |
| NOV2a | GADNCVSSPSEGIRKAKKGNYAFLWDVAVVEYAALTDDDCSVTVIGNSISSKGYGIALQH |
| NOV2b | GADNCVSSPSEGIRKAKKGNYAFLWDVAVVEYAALTDDDCSVTVIGNSISSKGYGIALQH |
| NOV2c | GADNCVSSPSEGIRKAKKGNYAFLWDVAVVEYAALTDDDCSVTVIGNSISSKGYGIALQH |
| NOV2d | GADNCVSSPSEGIRKAKKGNYAFLWDVAVVEYAALTDDDCSVTVIGNSISSKGYGIALQH |
| NOV2a | GSPYRDLFSQRILELQDTGDLDVLKQKWWPHMGRCDLTSHASAQADGKSLKLHSFAGVFC |
| NOV2b | GSPYRDLFSQRILELQDTGDLDVLKQKWWPHMGRCDLTSHASAQADGKSLKLHSFAGVFC |
| NOV2c | GSPYRDLFSQRILELQDTGDLDVLKQKWWPHMGRCDLTSHASAQADGKSLKLHSFAGVFC |
| NOV2d | GSPYRDLFSQRILELQDTGDLDVLKQKWWPHMGRCDLTSHASAQADGKSLKLHSFAGVFC |
| NOV2a | ILAIGLLLACLVAALELWWNSNRCHQETPKEDKEVNLEQVHRRMNSLMDEDIAHKQISPA |
| NOV2b | ILAIGLLLACLVAALELWWNSNRCHQETPKEDKEVNLEQVHRRMNSLMDEDIAHKQISPA |
| NOV2c | ILAIGLLLACLVAALELWWNSNRCHQETPKEDKEVNLEQVHRRMNSLMDEDIAHKQISPA |
| NOV2d | ILAIGLLLACLVAALELWWNSNRCHQETPKEDKEVNLEQVHRRMNSLMDEDIAHKQISPA |
| NOV2a | SIELSALEMGGLAPTQTLEPTREYQNTQLSVSTFLPEQSSHGTSRTLSSGPSSNLPLPLS |
| NOV2b | SIELSALEMGGLAPTQTLEPTREYQNTQLSVSTFLPEQSSHGTSRTLSSGPSSNLPLPLS |
| NOV2c | SIELSALEMGGLAPTQTLEPTREYQNTQLSVSTFLPEQSSHGTSRTLSSGPSSNLPLPLS |
| NOV2d | SIELSALEMGGLAPTQTLEPTREYQNTQLSVSTFLPEQSSHGTSRTLSSGPSSNLPLPLS |
| NOV2a | SSATMPSMQCKHRSPNGGLFRQSPVKTPIPMSFQPVPGGVLPEALDTSHGTSI--- |
| NOV2b | SSATMPSMQCKHRSPNGGLFRQSPVKTPIPMSFQPVPGGVLPEALDTSHGTSILEG |
| NOV2c | SSATMPSMQCKHRSPNGGLFRQSPVKTPIPMSFQPVPGGVLPEALDTSHGTSILEG |
| NOV2d | SSATMPSMQCKHRSPNGGLFRQSPVKTPIPMSFQPVPGGVLPEALDTSHGTSI--- |
| NOV2a | (SEQ ID NO: 6) |
| NOV2b | (SEQ ID NO: 8) |
| NOV2c | (SEQ ID NO: 10) |
| NOV2d | (SEQ ID NO: 12) |

Further analysis of the NOV2a protein yielded the following properties shown in Table 2C.

TABLE 2C
Protein Sequence Properties NOV2a

| | |
|---|---|
| SignalP analysis: | Cleavage site between residues 21 and 22 |
| PSORT II analysis: | |

| | |
|---|---|
| PSG: | a new signal peptide prediction method |
| | N-region: length 2; pos. chg 0; neg. chg 1 |
| | H-region: length 16; peak value 0.00 |
| | PSG score: −4.40 |
| GvH: | von Heijne's method for signal seq. recognition |
| | GvH score (threshold: −2.1): −1.56 |
| | possible cleavage site: between 17 and 18 |

>>>Seems to have no N-terminal signal peptide

| | |
|---|---|
| ALOM: | Klein et al's method for TM region allocation |
| | Init position for calculation: 1 |
| | Tentative number of TMS (s) for the threshold 0.5: 3 |
| | INTEGRAL Likelihood = −11.41 Transmembrane 567-583 |
| | INTEGRAL Likelihood = −0.43 Transmembrane 629-645 |
| | INTEGRAL Likelihood = −12.79 Transmembrane 834-850 |
| | PERIPHERAL Likelihood = 1.01 (at 437) |
| | ALOM score: −12.79 (number of TMSs: 3) |
| MTOP: | Prediction of membrane topology (Hartmann et al.) |
| | Center position for calculation: 574 |
| | Charge difference: 5.0 C(3.0) − N(−2.0) |
| | C > N: C-terminal side will be inside |

>>>membrane topology: type 3b

| MITDISC: | discrimination of mitochondrial targeting seq | | | |
|---|---|---|---|---|
| | R content: | 1 | Hyd Moment(75): | 3.64 |
| | Hyd Moment (95): | 6.21 | G content: | 0 |
| | D/E content: | 2 | S/T content: | 2 |
| | Score: −5.86 | | | |

Gavel: prediction of cleavage sites for mitochondrial preseq

R-2 motif at 29 VRA|DS

NUCDISC: discrimination of nuclear localization signals

| | |
|---|---|
| | pat4: none |
| | pat7: PKRYKGF (5) at 460 |
| | bipartite: RKWHSMASLNCIRKSTK at 341 |
| | content of basic residues: 9.5% |
| | NLS Score: 0.45 |
| KDEL: | ER retention motif in the C-terminus: none |

ER Membrane Retention Signals: none

| | |
|---|---|
| SKL: | peroxisomal targeting signal in the C-terminus: none |
| PTS2: | 2nd peroxisomal targeting signal: none |
| VAC: | possible vacuolar targeting motif: none |

RNA-binding motif: none
Actinin-type actin-binding motif:

| | |
|---|---|
| | type 1: none |
| | type 2: none |
| NMYR: | N-myristoylation pattern none |

Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

--------------------------
Final Results (k = 9/23):

44.4%: endoplasmic reticulum
22.2%: vacuolar
11.1%: Golgi
11.1%: nuclear
11.1%: mitochondrial

>> prediction for CG101396-01 is end (k = 9)

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE13278 | Human transporters and ion | 1 . . . 1009 | 1008/1009 (99%) | 0.0 |
| | channels (TRICH)-5 - Homo | 1 . . . 1009 | 1009/1009 (99%) | |
| | sapiens, 1009 aa. | | | |
| | [WO200177174-A2, 18-OCT- | | | |
| | 2001] | | | |
| AAM48984 | Rat glutamate receptor delta-1 | 1 . . . 1009 | 993/1009 (98%) | 0.0 |
| | subunit SEQ ID NO: 1 - Rattus | 1 . . . 1009 | 1004/1009 (99%) | |
| | norvegicus, 1009 aa. | | | |
| | [WO2002063 I 3-A2, 24-JAN- | | | |
| | 2002] | | | |
| AAG77969 | Human ion channel protein | 1 . . . 1009 | 999/1009 (99%) | 0.0 |
| | IC32391 - Homo sapiens, 1009 | 1 . . . 1009 | 1001/1009 (99%) | |
| | aa. [WO200183752-A2, 08- | | | |
| | NOV-2001] | | | |
| AAB40361 | Human ORFX ORF125 | 83 . . . 1009 | 927/927 (100%) | 0.0 |
| | polypeptide sequence SEQ ID | 1 . . . 927 | 927/927 (100%) | |
| | NO:250 - Homo sapiens, 927 aa. | | | |
| | [WO200058473-A2, 05-OCT- | | | |
| | 2000] | | | |
| AAM48988 | Human glutamate receptor delta- | 219 . . . 1009 | 791/791 (100%) | 0.0 |
| | 1 subunit SEQ ID NO: 8 - Homo | 1 . . . 791 | 791/791 (100%) | |
| | sapiens, 791 aa. | | | |
| | [WO200206313-A2, 24-JAN- | | | |
| | 2002] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q62640 | Glutamate receptor delta-1 | 1 . . . 1009 | 993/1009 (98%) | 0.0 |
| | subunit precursor - Rattus | 1 . . . 1009 | 1004/1009 (99%) | |
| | norvegicus (Rat), 1009 aa. | | | |
| CAD19379 | Sequence 4 from Patent | 1 . . . 1009 | 999/1009 (99%) | 0.0 |
| | WO0183752 - Homo sapiens | 1 . . . 1009 | 1001/1009 (99%) | |
| | (Human), 1009 aa. | | | |
| 528857 | glutamate receptor delta-1 | 1 . . . 1009 | 992/1009 (98%) | 0.0 |
| | chain precursor - rat, 1009 aa. | 1 . . . 1009 | 1004/1009 (99%) | |
| Q61627 | Glutamate receptor delta-1 | 1 . . . 1009 | 992/1009 (98%) | 0.0 |
| | subunit precursor - Mus | 1 . . . 1009 | 1002/1009 (98%) | |
| | musculus (Mouse), 1009 aa. | | | |
| Q9ULK0 | Hypothetical protein | 219 . . . 1009 | 791/791 (100%) | 0.0 |
| | KIAAI220 - Homo sapiens | 1 . . . 791 | 791/791 (100%) | |
| | (Human), 791 aa (fragment) | | | |

PFam analysis indicates that the NOV2a protein contains the domains shown in the Table 2F.

TABLE 2F
Domain Analysis of NOV2a

| | | Identities/ | |
|---|---|---|---|
| | NOV2a | Similarities | Expect |
| Pfam Domain | Match Region | for the Matched Region | Value |
| ANF₋receptor | 17 . . . 410 | 67/478 (14%) | 0.00034 |
| | | 261/478 (55%) | |
| SBP₋bac₋3 | 439 . . . 808 | 60/404 (15%) | 8.4e-07 |
| | | 221/404 (55%) | |
| lig₋chan | 562 . . . 852 | 123/325 (38%) | 6.9e-125 |
| | | 250/325 (77%) | |

### Example 3

The NOV3 clone was analyzed, and the nucleotide and encoded polypeptide sequences are shown in Table 3A.

TABLE 3A
NOV3 Sequence Analysis

| | | |
|---|---|---|
| NOV3a, CG102348-01 | SEQ ID NO: 15 | 3345 bp |

| | | |
|---|---|---|
| DNA Sequence | ORF Start: ATG at 18 | ORF Stop: TGA at 1479 |

CAGATGTCCAGTTCCAGATGCCTGGACCCAGAGTGTGGGGGAAATATCTCTGGAGAAGCCCTCACTCC
AAAGGCTGTCCAGGCGCAATGTGGTGGCTGCTTCTCTGGGGAGTCCTCCAGGCTTGCCCAACCCGGGG
CTCCGTCCTCTTGGCCCAAGAGCTACCCCAGCAGCTGACATCCCCCGGGTACCCAGAGCCGTATGGCA
AAGGCCAAGAGAGCAGCACGGACATCAAGGCTCCAGAGGGCTTTGCTGTGAGGCTCGTCTTCCAGGAC
TTCGACCTGGAGCCGTCCCAGGACTGTGCAGGGGACTCTGTCACAATCTCATTCGTCGGTTCGGATCC
AAGCCAGTTCTGTGGTCAGCAAGGCTCCCCTCTGGGCAGGCCCCCTGGTCAGAGGGAGTTTGTATCCT
CAGGGAGGAGTTTGCGGCTGACCTTCCGCACACAGCCTTCCTCGGAGAACAAGACTGCCCACCTTCAC
AAGGGCTTTCTGGCCCTCTACCAAACCGTGGCTGTGAACTATAGTCAGCCCATCAGCGAGGCCAGCAG
GGGCTCTGAGGCCATCAACGCACCTGGAGACAACCCTGCCAAGGTCCAGAACCACTGCCAGGAGCCCT
ATTATCAGGCCGCGGCAGCAGGGGCACTCACCTGTGCAACCCCAGGGACCTGGAAAGACAGACAGGAT
GGGGAGGAGGTTCTTCAGTGTATGCCTGTCTGCGGACGGCCAGTCACCCCCATTGCCCAGAATCAGAC
GACCCTCGGTTCTTCCAGAGCCAAGCTGGGCAACTTCCCCTGGCAAGCCTTCACCAGTATCCACGGCC
GTGGGGGCGGGGCCCTGCTGGGGGACAGATGGATCCTCACTGCTGCCCACACCGTCTACCCCAAGGAC
AGTGTTTCTCTCAGGAAGAACCAGAGTGTGAATGTGTTCTTGGGCCACACAGCCATAGATGAGATGCT
GAAACTGGGGAACCACCCTGTCCACCGTGTCGTTGTGCACCCCGACTACCGTCAGAATGAGTCCCATA
ACTTTAGCGGGGACATCGCCCTCCTGGAGCTGCAGCACAGCATCCCCCTGGGCCCCAACGTCCTCCCG
GTCTGTCTGCCCGATAATGAGACCCTCTACCGCAGCGGCTTGTTGGGCTACGTCAGTGGGTTTGGCAT
GGAGATGGGCTGGCTAACTACTGAGCTGAAGTACTCGAGGCTGCCTGTAGCTCCCAGGGAGGCCTGCA
ACGCCTGGCTCCAAAAGAGACAGAGACCCGAGGTGTTTTCTGACAATATGTTCTGTGTTGGGGATGAG
ACGCAAAGGCACAGTGTCTGCCAGGGGGACAGTGGCAGCGTCTATGTGGTATGGGACAATCATGCCCA
TCACTGGGTGGCCACGGGCATTGTGTCCTGGGGCATAGGGTGTGGCGAAGGGTATGACTTCTACACCA
AGGTGCTCAGCTATGTGGACTGGATCAAGGGAGTGATGAATGGCAAGAATTGACCCTGGGGGCTTGAA
CAGGGACTGACCAGCACAGTGGAGGCCCCAGGCAACAGAGGGCCTGGAGTGAGGACTGAACACTGGGG
TAGGGGTTGGGGGTGGGGGGTTGGGGGAGGCAGGGAAATCCTATTCACATCACTGTTGCACCAAGCCA
CTGCAAGAGAAACCCCCACCCGGCAAGCCCGCCCCATCCCAGACAGGAAGCAGAGTCCCACAGACCGC
TCCTCCTCACCCTCTACCTCCCTGTGCTCATGCACTAGGCCCCGGGAAGCCTGTACATCTCAACAACT
TTCGCCTTGAATGTCCTTAGAACCACCTTCCCCTACTTCATCTGTTGACACAGCTTTTATACTCACCT
GTGGAAGAGTCAGCTACTCACCCGCTATTAGAGTATGGAGGAAGGGGTTTTCATTGCATTGCATTTCT
GAAACATTCCTAAGACCCTTTAGTTGACCTTCAAATATTCAAGCTATTCTGCAGCTCCAAGATGCAAT
TATAGAAACAGCTCCTTTTTTATTTTATGTCCTCTATATGCCAGGTGCTTCACCTGTTATTTCACTTA
ATCCTCATACCATATTTGCAAAGGATGTGTTATTATCTATGTGTGACAAATGAGGAAACTGAGGCTCA
GGGGATAAAGGGACTTGCCCAAGTCCCACAGCTGGTGTGTGACTGCAGAGACTGTGCTCTTCCCAGTG
TGCTGCAATACTTCTCAACCCTCCTCTAACCTGCTGTGTCACCCGCTTTCCCTCCCAGCCCCCACATC
CTTACCATTTTCCCTCCCTGGGAATTCCTGCTTCTGCGAAAATGGTATCCTCTAGCTCACACTTTCCT
AATGGCCCCATCTCCTGCAGAAGCCAGGTGAGCCCAGCACTGGACTGAAGTTCTTGCAGACACCCCAC
CTGTGCCCCTATCATCAGGGGAACTGCTCCACCTGAGAGGACCAACTCTTTAATTTTTAGTAAAACCT
GAAGGTGATGGGCCGGGCGCAGTGGCTCACGCCTGTAATCCCAACACCTTAGGAGTCCGAGGTGGGTG
GATCACGAGGTCAGGAGATCCAGCCCATCCTGGCCAACATGGTGAAACCCCATCTCTACTAAAAATAC
AAAAATTAGCCGGGCGTGGTGACACGTGCCTGTAGTCCCAGCTACTCGGGAGGCTGAGGCAGGAGAAT
CACTTGAACCTGGGAGGCGGAGGTTGCAGTGAGCTAAGATCACGCCACTGCACTCCAGCCTGCGGACA
GACCAAGACTTCATCCCCCCCAAAAAAAAAAGATTGGAGGTGATTTACAGTGAAAGACACAAATAAAA
TACAACTGTTCAATGGAAATAGAAAATAAACACCATAAAAGAGAGAAGAGAGGTAATTTGTTAGCATC
AAGAGTCAAGTTGCTATATGGTCAAAGGTTAAATTTATCTCTAAAAAATGGCAGGATTCAAAGTTGTA
CATACATGTGATTACTTCTGTTTTTTACACCCACATACAGTACAAAAGATTATTAAAAATATTCCCAA
AAGGCAGGTGCAATGATGCACACTTATACCCCCAGCCACTCAGGAGGCTGATGCAAGAGGATCGCTTG
AGCCCAGGAGTTGAAGTCCAGCCTAAGCAACATAGTGAAACCCCATCTCCAAAAATATAATAATAATT
CTCTCAAAATACTAAACAGAGGTGGTTTTATTGATAAGATTTTGGCTGTTTGGTTTTCCACTATTCTC
TATTGGCTAAAATTTGTTTAATGAGCATGAAATGTTTTTATTTTATTTTGCTTATTTTTATGATTGCA
AAAAATGATATGAGTTTCTCCCTGCCAAGGCAAAAAATATATATATATACCTATAAAAAAAAAAAAAA
AAAAAAAAAAAAA

NOV3a, CG102348-01

| | | | |
|---|---|---|---|
| Protein Sequence | SEQ ID NO: 16 | 487 aa | MW at 53483.9kD |

MPGPRVWGKYLWRSPHSKGCPGANWWLLLWGVLQACPTRGSVLLAQELPQQLTSPGYPEPYGKGQESS
TDIKAPEGFAVRLVFQDFDLEPSQDCAGDSVTISFVGSDPSQFCGQQGSPLGRPPGQREFVSSGRSLR
LTFRTQPSSENKTAHLHKGFLALYQTVAVNYSQPISEASRGSEAINAPGDNPAKVQNHCQEPYYQAAA
AGALTCATPGTWKDRQDGEEVLQCMPVCGRPVTPIAQNQTTLGSSRAKLGNFPWQAFTSIHGRGGGAL
LGDRWILTAAHTVYPKDSVSLRKMQSVNVFLGHTAIDEMLKLGNHPVHRVVVHPDYRQNESHNFSGDI
ALLELQHSIPLGPNVLPVCLPDNETLYRSGLLGYVSGFGMEMGWLTTELKYSRLPVAPREACNAWLQK
RQRPEVFSDNMFCVGDETQRHSVCQGDSGSVYVVWDNHAHHWVATGIVSWGIGCGEGYDFYTKVLSYV
DWIKGVNNGKN

| | | |
|---|---|---|
| NOV3b, 199842645 | SEQ ID NO: 17 | 1368 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGTTCGTCTTCCAGGACTTCGACCTGGAGTCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCACGGCAGCAGGGGCACTCACCTGTGCAACCCCAGGGA
CCTGGAAAGACAGACAGGATGGGGAGGAGGTTCTTCAGTGTATGCCTGTCTGCGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCAGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGTTGGGGATGAGACGCAAAGGCACAGTGTCTGCCAGGGGGACAGTGGCAGCGTCTATGTG
GTATGGGACAATCATGCCCATCACTGGGTGGCCACGGGCATTGTGTCcTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3b, 199842645

| | | | |
|---|---|---|---|
| Protein Sequence | SEQ ID NO: 18 | 456 aa | MW at 49986.8kD |

KLCPTRGSVLLAQELPQQLTSPGYPEPYGKGQESSTDIKAPEGFAVRFVFQDFDLESSQDCAGDSVTI
SFVGSDPSQFCGQQGSPLGRPPGQREFVSSGRSLRLTFRTQPSSENKTAHLHKGFLALYQTVAVNYSQ
PISEASRGSEAINAPGDNPAKVQNHCQEPYYQATAAGALTCATPGTWKDRQDGEEVLQCMPVCGRPVT
PIAQNQTTLGSSRAKLGNFPWQAFTSIHGRGGGALLGDRWILTAAHTIYPKDSVSLRKNQSVNVFLGH
TAIDEMLKLGNHPVHRVVVHPDYRQNESHNFSGDIALLELQHSIPLGPNVLPVCLPDNETLYRSGLLG
YVSGFGMEMGWLTTELKYSRLPVAPREACNAWLQKRQRPEVFSDNMFCVGDETQRRSVCQGDSGSVYV
VWDNHAHHWVATGIVSWGIGCGEGYDFYTKVLSYVDWIKGVMNGKNKL

| | | |
|---|---|---|
| NOV3c, 198306343 | SEQ ID NO: 19 | 741 bp |

| | | |
|---|---|---|
| DNA Sequence | ORF Start: at 1 | ORF Stop: end of sequence |

AAGCTTACCCTCGGTTCTTCCAGAGCCAAGCTGGGCAACTTCCCCTGGCAAGCCTTCACCAGTATCCA
CGGCCGTGGGGGCGGGGCCCTGCTGGGGGACAGATGGATCCTCACTGCTGCCCACACCATCTACCCCA
AGAACAGTGTTTCTCTCAGGAAGAACCAGAGTGTGAATGTGTTCTTGGGCCACACAGCCATAGATGAG
ATGCTGAAACTGGGGAACCACCCTGTCCACCGTGTCGTTGTGCACCCCGACTACCGTCAGAATGAGTC
CCATAACTTTAGCGGGGACATCGCCCTCCTGGAGCTGCAGCACAGCATCCCCCTGGGCCCCAACGTCC
TCCCGGTCTGTCTGCCCGATAATGAGACCCTCTACCGCAGCGGCTTGTTGGGCTACGTCAGTGGGTTT
GGCATGGAGATGGGCTGGCTAACTACTGAGCTGAAGTACTCGAGGCTGCCTGTAGCTCCCAGGGAGGC
CTGCAACGCCTGGCTCCAAAAGAGACAGAGACCCGAGGTGTTTTCTGACAATATGTTCTGTGTTGGGG
ATGAGACGCAAAGGCACAGTGTCTGCCAGGGGGACAGTGGCAGCGTCTATGTGGTATGGGACAATCGT
GCCCATCACTGGGTGGCCACGGGCATTGTGTCCTGGGGCATAGGGTGTGGCGAAGGGTATGACTTCTA
CACCAAGGTGCTCAGCTATGTGGACTGGATCAAGGGAGTGATGAATGGCAAGAATAAGCTT

NOV3c, 198306343

| | | | |
|---|---|---|---|
| Protein Sequence | SEQ ID NO: 20 | 247 aa | MW at 27566.2kD |

KLTLGSSRAKLGNFPWQAFTSIHGRGGGALLGDRWILTAAHTIYPKNSVSLRKNQSVNVFLGHTAIDE
MLKLGNHPVHRVVVHPDYRQNESHNFSGDIALLELQHSIPLGPNVLPVCLPDNETLYRSGLLGYVSGF
GMEMGWLTTELKYSRLPVAPREACNAWLQKRQRPEVFSDNNFCVGDETQRHSVCQGDSGSVYVVWDNR
AHHWVATGIVSWGIGCGEGYDFYTKVLSYVDWIKGVMNGKNKL

| | | |
|---|---|---|
| NOV3d, 199842665 | SEQ ID NO: 21 | 1368 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCGCGGCAGCAGGGGCACTCACCTGTGCAACCCCAGGGA
CCTGGAAAGACAGACAGGATGGGGAGGAGGTTCTTCAGTGTATGCCTGTCTGCGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCGTCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCAGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGTTGGGGATGAGACGCAAAGGCACAGTGTCTGCCAGGGGGACAGTGGCAGCGTCTATGTG
GTATGGGACAATCATGCCCATCACTGGGTGGCCACGGGCATTGTGTCCTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3d, 199842665

| | | | |
|---|---|---|---|
| Protein Sequence | SEQ ID NO: 22 | 456 aa | MW at 49918.7kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSENKTAHLHKGFLALYQTVAVNYSQ
PISEASRGSEAINAPGDNPAKVQNHCQEPYYQAAAAGALTCATPGTWKDRQDGEEVLQCMPVCGRPVT
PIAQNQTTLGSSRAKLGNFPWQAFTSIHGRGGGALLGDRWILTAAHTVYPKDSVSLRKNQSVNVFLGH
TAIDEMLKLGNHPVHRVVVHPDYRQNESHNFSGDIALLELQHSIPLGPNVLPVCLPDNETLYRSGLLG
YVSGFGMEMGWLTTELKYSRLPVAPREACNAWLQKRQRPEVFSDNMFCVGDETQRHSVCQGDSGSVYV
VWDNHAHHWVATGIVSWGIGCGEGYDFYTKVLSYVDWIKGVMNGKNKL

| | | |
|---|---|---|
| NOV3e, 199842661 | SEQ ID NO: 23 | 1368 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGG
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCGCGGCAGCAGGGGCACTCACCTGTGCAACCCCAGGGA
CCTGGAAAGACAGACAGGATGGGGAGGAGGTTCTTCAGTGTATGCCTGTCTGCGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCAGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGITGGGGATGAGACGCAAAGGCACAGTGTCTGCCAGGGGGACAGTGGCAGCGTCTATGTG
GTATGGGACAATCATGCCCATCACTGGGTGGCCACGGGCATTGTGTCCTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3e, 199842661

| | | | |
|---|---|---|---|
| Protein Sequence | SEQ ID NO: 24 | 456 aa | MW at 49933.7kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSEDKTAHLHKGFLALYQTVAVNYSQ
PISEASRGSEAINAPGDNPAKVQNHCQEPYYQAAAAGALTCATPGTWKDRQDGEEVLQCMPVCGRPVT
PIAQNQTTLGSSRAKLGNFPWQAFTSIHGRGGGALLGDRWILTAAHTIYPKDSVSLRKNQSVNVFLGH
TAIDEMLKLGNHPVHRVVVHPDYRQNESHNFSGDIALLELQHSIPLGPNVLPVCLPDNETLYRSGLLG
YVSGFGMEMGWLTTELKYSRLPVAPREACNAWLQKRQRPEVFSDNNFCVGDETQRHSVCQGDSGSVYV
VWDNHAHHWVATGIVSWGIGCGEGYDFYTKVLSYVDWIKGVMNGKNKL

| | | |
|---|---|---|
| NOV3f, 199597024 | SEQ ID NO: 25 | 1479 bp |

| | | |
|---|---|---|
| DNA Sequence | ORF Start: at 1 | ORF Stop: end of sequence |

AAGCTTCCCACCATGCCTGGACCCAGAGTGTGGGGGAAATATCTCTGGAGAAGCCCTCACTCCAAAGG
CTGTCCAGGCGCAATGTGGTGGCTGCTTCTCTGGGGAGTCCTCCAGGCTTGCCCAACCCGGGGCTCCG
TCCTCTTGGCCCAAGAGCTACCCCAGCAGCTGACATCCCCCGGGTACCCAGAGCCGTATGGCAAAGGC
CAAGAGAGCAGCACGGACATCAAGGCTCCAGAGGGCTTTGCTGTGAGGCTCGTCTTCCAGGACTTCGA
CCTGGAGCCGTCCCAGGACTGTGCAGGGGACTCTGTCACAGTCTCATTCGTCGGTTCGGATCCAAGCC
AGTTCTGTGGTCAGCAAGGCTCCCCTCTGGGCAGGCCCCCTGGTCAGAGGGAGTTTGTATCCTCAGGG
AGGAGTTTGCGGCTGACCTTCCGCACACAGCCTTCCTCGGAGAACAAGACTGCCCACCTCCACAAGGG
CTTCCTGGCCCTCTACCAAACCGTGGCTGTGAACTATAGTCAGCCCATCAGCGAGGCCAGCAGGGGCT
CTGAGGCCATCAACGCACCTGGAGACAACCCTGCCAAGGTCCAGAACCACTGCCAGGAGCCCTATTAT
CAGGCCGCGGCAGCAGGGGCACTCACCTGTGCAACCCCAGGGACCTGGAAAGACAGACAGGATGGGGA
GGAGGTTCTTCAGTGTATGCCTGTCTGCGGACGGCCAGTCACCCCCATTGCCCAGAATCAGACGACCC
TCGGTTCTTCCAGAGCCAAGCTGGGCAACTTCCCCTGGCAAGCCTTCACCAGTATCCACGGCCGTGGG
GGCGGGGCCCTGCTGGGGGACAGATGGATCCTCACTGCTGCCCACACCATCTACCCCAAGGACAGTGT
TTCTCTCAGGAAGAACCAGAGTGTGAATGTGTTCTTGGGCCACACAGCCATAGATGAGATGCTGAAAC
TGGGGAACCACCCTGTCCACCGTGTCGTTGTGCACCCCGACTACCGTCAGAATGAGTCCCATAACTTT
AGCGGGGACATCGCCCTCCTGGAGCTGCAGCACAGCATCCCCCTGGGCCCCAACGTCCTCCCGGTCTG
TCTGCCCGATAATGAGACCCTCTACCGCAGCGGCTTGTTGGGCTACGTCAGTGGGTTTGGCATGGAGA
TGGGCTGGCTAACTACTGAGCTGAAGTACTCGAGGCTGCCTGTAGCTCCCAGGGAGGCCTGCAACGCC
TGGCTCCAAAAGAGACAGAGACCCGAGGTGTTTTCTGACAATATGTTCTGTGTTGGGGATGAGACGCA
AAGGCACAGTGTCTGCCAGGGGGACAGTGGCAGCGTCTATGTGGTATGGGACAATCATGCCCATCACT
GGGTGGCCACGGGCATTGTGTCCTGGGGCATAGGGTGTGGCGAAGGGTATGACTTCTACACCAAGGTG
CTCAGCTATGTGGACTGGATCAAGGGAGTGATGAATGGCAAGAATAAGCTT

NOV3f, 199597024

| | | | |
|---|---|---|---|
| Protein Sequence | SEQ ID NO: 26 | 493 aa | MW at 54164.8kD |

KLPTMPGPRVWGKYLWRSPHSKGCPGAMWWLLLWGVLQACPTRGSVLLAQELPQQLTSPGYPEPYGKG
QESSTDIKAPEGFAVRLVFQDFDLEPSQDCAGDSVTVSFVGSDPSQFCGQQGSPLGRPPGQREFVSSG
RSLRLTFRTQPSSENKTAHLHKGFLALYQTVAVNYSQPISEASRGSEAINAPGDNPAKVQNHCQEPYY
QAAAAGALTCATPGTWKDRQDGEEVLQCMPVCGRPVTPIAQNQTTLGSSRAKLGNFPWQAFTSIHGRG
GGALLGDRWILTAAHTIYPKDSVSLRKNQSVNVFLGHTAIDEMLKLGNHPVHRVVVHPDYRQNESHNF
SGDIALLELQHSIPLGPNVLPVCLPDNETLYRSGLLGYVSGFGMEMGWLTTELKYSRLPVAPREACNA
WLQKRQRPEVFSDNMFCVGDETQRHSVCQGDSGSVYVVWDNHAHHWVATGIVSWGIGCGEGYDFYTKV
LSYVDWIKGVMNGKNKL

| | | |
|---|---|---|
| NOV3g, 199842653 | SEQ ID NO: 27 | 1293 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCGCGGCAGCAGTCTGCGGACGGCCAGTCACCCCCATTG
CCCAGAATCAGACGACCCTCGGTTCTTCCAGAGCCAAGCTGGGCAACTTCCCCTGGCAAGCCTTCACC
AGTATCCACGGCCGTGGGGGCGGGGCCCTGCTGGGGGACAGATGGATCCTCACTGCTGCCCACACCAT
CTACCCCAAGGACAGTGTTTCTCTCAGGAAGAACCAGAGTGTGAATGTGTTCTTGGGCCACACAGCCA
TAGATGAGATGCTGAAACTGGGGAACCACCCTGTCCACCGTGTCGTTGTGCACCCCGACTACCGTCAG
AATGAGTCCCATAACTTTAGCGGGGACATCGCCCTCCTGGAGCTGCAGCACAGCATCCCCCTGGGCCC
CAACGTCCTCCCGGTCTGTCTGCCCGATAATGAGACCCTCTACCGCAGCGGCTTGTTGGGCTACGTCA
GTGGGTTTGGCATGGAGATGGGCCGGCTAACTACTGAGCTGAAGTACTCGAGGCTGCCTGTAGCTCCC
AGGGAGGCCTGCAACGCCTGGCTCCAAAAGAGACAGAGACCCGAGGTGTTTTCTGACAATATGTTCTG
TGTCGGGGATGAGACGCAAAGGCACAGTGTCTGCCAGGGGGACAGTGGCAGCGTCTATGTGGTATGGG
ACAATCATGCCCATCACTGGGTGGCCACGGGCATTGTGTCCTGGGGCATAGGGTGTGGCGAAGGGTAT
GACTTCTACACCAAGGTGCTCAGCTATGTGGACTGGATCAAGGGAGTGATGAATGGCAAGAATAAGCT
T

NOV3g, 199842653

| | | | |
|---|---|---|---|
| Protein Sequence | SEQ ID NO: 28 | 431 aa | MW at 47213.7kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSENKTAHLHKGFLALYQTVAVNYSQ
PISEASRGSEAINAPGDNPAKVQNHCQEPYYQAAAAVCGRPVTPIAQNQTTLGSSRAKLGNFPWQAFT
SIHGRGGGALLGDRWILTAAHTIYPKDSVSLRKNQSVNVFLGHTAIDEMLKLGNHPVHRVVVHPDYRQ
NESHNFSGDIALLELQHSIPLGPNVLPVCLPDNETLYRSGLLGYVSGFGMEMGRLTTELKYSRLPVAP
REACNAWLQKRQRPEVFSDNMFCVGDETQRHSVCQGDSGSVYVVWDNHAHHWVATGIVSWGIGCGEGY
DFYTKVLSYVDWIKGVMNGKNKL

| | | |
|---|---|---|
| NOV3h, 199652830 | SEQ ID NO: 29 | 1368 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCCGCCAAGGTCCA
GAACCACTGCCAGGAGCCCTATTATCAGGCCGCGGCAGCAGGGGCACTCACCTGTGCAACCCCAGGGA
CCTGGGAAGACAGACAGGATGGGGAAGAGGTTCTTCAGTGTATGCCTGTCTGTGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCAGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGTTGGGGATGAGACGCAAAGGCACAGTGTCTGCCAGGGGGACAGTGGCAGCGTCTATGTG
GTATGGGACAATCATGCCCATGACTGGGTGGCCACGGGCATTGTGTCCTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3h, 199652830

| | | | |
|---|---|---|---|
| Protein Sequence | SEQ ID NO: 30 | 456 aa | MW at 49933.7kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSENKTAHLHKGFLALYQTVAVNYSQ
PISEASRGSEAINAPGDNPAKVQNHCQEPYYQAAAAGALTCATPGTWEDRQDGEEVLQCMPVCGRPVT
PIAQNQTTLGSSRAKLGNFPWQAFTSIHGRGGGALLGDRWILTAAHTIYPKDSVSLRKNQSVNVFLGH
TAIDEMLKLGNHPVHRVVVHPDYRQNESHNFSGDIALLELQHSIPLGPNVLPVCLPDNETLYRSGLLG
YVSGFGMEMGWLTTELKYSRLPVAPREACNAWLQKRQRPEVFSDNMFCVGDETQRHSVCQGDSGSVYV
VWDNHANHWVATGIVSWGIGCGEGYDFYTKVLSYVDWIKGVMNGKNKNKL

| | | |
|---|---|---|
| NOV3i, 199652835 | SEQ ID NO: 31 | 1368 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCGCGGCAGCAGGGGCACTCACCTGTGCAACCCCAGGGA
CCTGGAAAGACAGACAGGATGGGGAGGAGGTTCTTCAGTGTATGCCTGTCTGCGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCGGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGTTGGGGATGGGACACAAAGGCACAGTGTCTGCCAGGGGGACAGTGGCAGCGTCTATGTG
GTATGGGACAATCATGCCCATCACTGGGTGGCCACGGGCATTGTGTCCTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3i, 199652835

| | | | |
|---|---|---|---|
| Protein Sequence | SEQ ID NO: 32 | 456 aa | MW at 49888.7kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSENKTAHLHKGFLALYQTVAVNYSQ
PISEASRGSEAINAPGDNPAKVQNHCQEPYYQAAAAGALTCATPGTWKDRQDGEEVLQCMPVCGRPVT
PIAQNQTTLGSSRAKLGNFPWQAFTSIHGRGGGALLGDRWILTAAHTIYPKDSVSLRKMQSVNVFLGH
TAIDEMLKLGNHPVHRVVVHPDYRQNESHNFSGDIALLELRHSIPLGPNVLPVCLPDNETLYRSGLLG
YVSGFGMEMGWLTTELKYSRLPVAPREACNAWLQKRQRPEVFSDNMFCVGDGTQRHSVCQGDSGSVYV
VWDNHAHHWVATGIVSWGIGCGEGYDFYTKVLSYVDWIKGVMNGKNKL

| | | |
|---|---|---|
| NOV3j, 198306308 | SEQ ID NO: 33 | 387 bp |

| | | |
|---|---|---|
| DNA Sequence | ORF Start: at 1 | ORF Stop: end of sequence |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCGGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTTCACAAGGGCTTTCTGGCCCTCTACAAGCTT

NOV3j, 198306308

| | | | |
|---|---|---|---|
| Protein Sequence | SEQ ID NO: 34 | 129 aa | MW at 14001.5kD |

KLCPTRGSVLLAQELPQQLTSPGYPEPYGKGQESSTDIKAPEGFAVRLVFQDFDLEPSQDCAGDSVTI
SFVGSDPSQFCGQQGSPLGRPPGQREFVSSGRSLRLTFRTQPSSENKTAHLHKGFLALYKL

| | | |
|---|---|---|
| NOV3k, CG102348-02 | SEQ ID NO: 35 | 387 bp |

| | | |
|---|---|---|
| DNA Sequence | ORF Start: at 7 | ORF Stop: at 382 |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCGGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTTCACAAGGGCTTTCTGGCCCTCTACAAGCTT

NOV3k, CG102348-02

| | | | |
|---|---|---|---|
| Protein Sequence | SEQ ID NO: 36 | 125 aa | MW at 13518.9kD |

CPTRGSVLLAQELPQQLTSPGYPEPYGKGQESSTDIKAPEGFAVRLVFQDFDLEPSQDCAGDSVTISF
VGSDPSQFCGQQGSPLGRPPGQREFVSSGRSLRLTFRTQPSSENKTAHLHKGFLALY

| | | |
|---|---|---|
| NOV3l, CG102348-03 | SEQ ID NO: 37 | 741 bp |

| | | |
|---|---|---|
| DNA Sequence | ORF Start: at 7 | ORF Stop: at 736 |

AAGCTTACCCTCGGTTCTTCCAGAGCCAAGCTGGGCAACTTCCCCTGGCAAGCCTTCACCAGTATCCA
CGGCCGTGGGGGCGGGGCCCTGCTGGGGGACAGATGGATCCTCACTGCTGCCCACACCATCTACCCCA
AGAACAGTGTTTCTCTCAGGAAGAACCAGAGTGTGAATGTGTTCTTGGGCCACACAGCCATAGATGAG
ATGCTGAAACTGGGGAACCACCCTGTCCACCGTGTCGTTGTGCACCCCGACTACCGTCAGAATGAGTC
CCATAACTTTAGCGGGGACATCGCCCTCCTGGAGCTGCAGCACAGCATCCCCCTGGGCCCCAACGTCC
TCCCGGTCTGTCTGCCCGATAATGAGACCCTCTACCGCAGCGGCTTGTTGGGCTACGTCAGTGGGTTT
GGCATGGAGATGGGCTGGCTAACTACTGAGCTGAAGTACTCGAGGCTGCCTGTAGCTCCCAGGGAGGC
CTGCAACGCCTGGCTCCAAAAGAGACAGAGACCCGAGGTGTTTTCTGACAATATGTTCTGTGTTGGGG
ATGAGACGCAAAGGCACAGTGTCTGCCAGGGGGACAGTGGCAGCGTCTATGTGGTATGGGACAATCGT
GCCCATCACTGGGTGGCCACGGGCATTGTGTCCTGGGGCATAGGGTGTGGCGAAGGGTATGACTTCTA
CACCAAGGTGCTCAGCTATGTGGACTGGATCAAGGGAGTGATGAATGGCAAGAATAAGCTT

NOV3l, CG102348-03

| | | | |
|---|---|---|---|
| Protein Sequence | SEQ ID NO: 38 | 243 aa | MW at 27083.5kD |

TLGSSRAKLGNFPWQAFTSIHGRGGGALLGDRWILTAAHTIYPKNSVSLRKNQSVNVFLGHTAIDEML
KLGNHPVHRVVVHPDYRQNESHNFSGDIALLELQHSIPLGPNVLPVCLPDNETLYRSGLLGYVSGFGM
EMGWLTTELKYSRLPVAPREACNAWLQKRQRPEVFSDNMFCVGDETQRHSVCQGDSGSVYVVWDNRAH
HWVATGIVSWGIGCGEGYDFYTKVLSYVDWIKGVMNGKN

| | | |
|---|---|---|
| NOV3m, CG102348-04 | SEQ ID NO: 39 | 1293 bp |

| | | |
|---|---|---|
| DNA Sequence | ORF Start: at 7 | ORF Stop: at 1288 |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGCTCGTCTTCCAGGACTTCGACCTGGAGCCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCGCGGCAGCAGTCTGCGGACGGCCAGTCACCCCCATTG
CCCAGAATCAGACGACCCTCGGTTCTTCCAGAGCCAAGCTGGGCAACTTCCCCTGGCAAGCCTTCACC
AGTATCCACGGCCGTGGGGGCGGGGCCCTGCTGGGGGACAGATGGATCCTCACTGCTGCCCACACCAT
CTACCCCAAGGACAGTGTTTCTCTCAGGAAGAACCAGAGTGTGAATGTGTTCTTGGGCCACACAGCCA
TAGATGAGATGCTGAAACTGGGGAACCACCCTGTCCACCGTGTCGTTGTGCACCCCGACTACCGTCAG
AATGAGTCCCATAACTTTAGCGGGGACATCGCCCTCCTGGAGCTGCAGCACAGCATCCCCCTGGGCCC
CAACGTCCTCCCGGTCTGTCTGCCCGATAATGAGACCCTCTACCGCAGCGGCTTGTTGGGCTACGTCA
GTGGGTTTGGCATGGAGATGGGCCGGCTAACTACTGAGCTGAAGTACTCGAGGCTGCCTGTAGCTCCC
AGGGAGGCCTGCAACGCCTGGCTCCAAAAGAGACAGAGACCCGAGGTGTTTTCTGACAATATGTTCTG
TGTCGGGGATGAGACGCAAAGGCACAGTGTCrGCCAGGGGGACAGTGGCAGCGTCTATGTGGTATGGG
ACAATCATGCCCATCACTGGGTGGCCACGGGCATTGTGTCCTGGGGCATAGGGTGTGGCGAAGGGTAT
GACTTCTACACCAAGGTGCTCAGCTATGTGGACTGGATCAAGGGAGTGATGAATGGCAAGAATAAGCT
T

NOV3m, CG102348-04

| | | | |
|---|---|---|---|
| Protein Sequence | SEQ ID NO: 40 | 427 aa | MW at 46731.0kD |

CPTRGSVLLAQELPQQLTSPGYPEPYGKGQESSTDIKAPEGFAVRLVFQDFDLEPSQDCAGDSVTISF
VGSDPSQFCGOQGSPLGRPPGQREFVSSGRSLRLTFRTQPSSENKTAHLHKGFLALYQTVAVNYSQPI
SEASRGSEAINAPGDNPAKVQNHCQEPYYQAAAAVCGRPVTPIAQNQTTLGSSRAKLGNFPWQAFTSI
HGRGGGALLGDRWILTAAHTIYPKDSVSLRKNQSVNVFLGHTAIDEMLKLGNHPVHRVVVHPDYRQNE
SHNFSGDIALLELQHSIPLGPNVLPVCLPDNETLYRSGLLGYVSGFGMEMGRLTTELKYSRLPVAPRE
ACNAWLQKRQRPEVFSDNMFCVGDETQRHSVCQGDSGSVYVVWDNHAHHWVATGIVSWGIGCGEGYDF
YTKVLSYVDWIKGVMNGKN

| | | |
|---|---|---|
| NOV3n, CG102348-05 | SEQ ID NO: 41 | 1368 bp |

| | | |
|---|---|---|
| DNA Sequence | ORF Start: at 7 | ORF Stop: at 1363 |

AAGCTTTGCCCAACCCGGGGCTCCGTCCTCTTGGCCCAAGAGCTACCCCAGCAGCTGACATCCCCCGG
GTACCCAGAGCCGTATGGCAAAGGCCAAGAGAGCAGCACGGACATCAAGGCTCCAGAGGGCTTTGCTG
TGAGGTTCGTCTTCCAGGACTTCGACCTGGAGTCGTCCCAGGACTGTGCAGGGGACTCTGTCACAATC
TCATTCGTCGGTTCGGATCCAAGCCAGTTCTGTGGTCAGCAAGGCTCCCCTCTGGGCAGGCCCCCTGG
TCAGAGGGAGTTTGTATCCTCAGGGAGGAGTTTGCGGCTGACCTTCCGCACACAGCCTTCCTCGGAGA
ACAAGACTGCCCACCTCCACAAGGGCTTCCTGGCCCTCTACCAAACCGTGGCTGTGAACTATAGTCAG
CCCATCAGCGAGGCCAGCAGGGGCTCTGAGGCCATCAACGCACCTGGAGACAACCCTGCCAAGGTCCA
GAACCACTGCCAGGAGCCCTATTATCAGGCCACGGCAGCAGGGGCACTCACCTGTGCAACCCCAGGGA
CCTGGAAAGACAGACAGGATGGGGAGGAGGTTCTTCAGTGTATGCCTGTCTGCGGACGGCCAGTCACC
CCCATTGCCCAGAATCAGACGACCCTCGGTTCTTCCAGAGCCAAGCTGGGCAACTTCCCCTGGCAAGC
CTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCAC
ACAGCCATAGATGAGATGCTGAAACTGGGGAACCACCCTGTCCACCGTGTCGTTGTGCACCCCGACTA
CCGTCAGAATGAGTCCCATAACTTTAGCGGGGACATCGCCCTCCTGGAGCTGCAGCACAGCATCCCCC
TGGGCCCCAACGTCCTCCCGGTCTGTCTGCCCGATAATGAGACCCTCTACCGCAGCGGCTTGTTGGGC
TACGTCAGTGGGTTTGGCATGGAGATGGGCTGGCTAACTACTGAGCTGAAGTACTCGAGGCTGCCTGT
AGCTCCCAGGGAGGCCTGCAACGCCTGGCTCCAAAAGAGACAGAGACCCGAGGTGTTTTCTGACAATA
TGTTCTGTGTTGGGGATGAGACGCAAAGGCACAGTGTCTGCCAGGGGGACAGTGGCAGCGTCTATGTG
GTATGGGACAATCATGCCCATCACTGGGTGGCCACGGGCATTGTGTCCTGGGGCATAGGGTGTGGCGA
AGGGTATGACTTCTACACCAAGGTGCTCAGCTATGTGGACTGGATCAAGGGAGTGATGAATGGCAAGA
ATAAGCTT

NOV3n, CG102348-05

| | | | |
|---|---|---|---|
| Protein Sequence | SEQ ID NO: 42 | 452 aa | MW at 49504.1kD |

CPTRGSVLLAQELPQQLTSPGYPEPYGKGQESSTDIKAPEGFAVRFVFQDFDLESSQDCAGDSVTISF
VGSDPSQFCGQQGSPLGRPPGQREFVSSGRSLRLTFRTQPSSENKTAHLHKGFLALYQTVAVNYSQPI
SEASRGSEAINAPGDNPAKVQNHCQEPYYQATAAGALTCATPGTWKDRQDGEEVLQCMPVCGRPVTPI
AQNQTTLGSSRAKLGNFPWQAFTSIHGRGGGALLGDRWILTAAHTIYPKDSVSLRKNQSVNVFLGHTA
IDEMLKLGNHPVHRVVVHPDYRQNESHNFSGDIALLELQHSIPLGPNVLPVCLPDNETLYRSGLLGYV
SGFGMEMGWLTTELKYSRLPVAPREACNAWLQKRQRPEVFSDNNFCVGDETQRHSVCQGDSGSVYVVW
DNHAHHWVATGIVSWGIGCGEGYDFYTKVLSYVDWIKGVMNGKN

| | | |
|---|---|---|
| NOV3o, CG102348-06 | SEQ ID NO: 43 | 1479 bp |

| | | |
|---|---|---|
| DNA Sequence | ORF Start: ATG at 13 | ORF Stop: at 1474 |

AAGCTTCCCACCATGCCTGGACCCAGAGTGTGGGGGAAATATCTCTGGAGAAGCCCTCACTCCAAAGG
CTGTCCAGGCGCAATGTGGTGGCTGCTTCTCTGGGGAGTCCTCCAGGCTTGCCCAACCCGGGGCTCCG
TCCTCTTGGCCCAAGAGCTACCCCAGCAGCTGACATCCCCCGGGTACCCAGAGCCGTATGGCAAAGGC
CAAGAGAGCAGCACGGACATCAAGGCTCCAGAGGGCTTTGCTGTGAGGCTCGTCTTCCAGGACTTCGA
CCTGGAGCCGTCCCAGGACTGTGCAGGGGACTCTGTCACAATCTCATTCGTCGGTTCGGATCCAAGCC
AGTTCTGTGGTCAGCAAGGCTCCCCTCTGGGCAGGCCCCCTGGTCAGAGGGAGTTTGTATCCTCAGGG
AGGAGTTTGCGGCTGACCTTCCGCACACAGCCTTCCTCGGAGAACAAGACTGCCCACCTCCACAAGGG
CTTCCTGGCCCTCTACCAAACCGTGGCTGTGAACTATAGTCAGCCCATCAGCGAGGCCAGCAGGGGCT
CTGAGGCCATCAACGCACCTGGAGACAACCCTGCCAAGGTCCAGAACCACTGCCAGGAGCCCTATTAT
CAGGCCACGGCAGCAGGGGCACTCACCTGTGCAACCCCAGGGACCTGGAAAGACAGACAGGATGGGGA
GGAGGTTCTTCAGTGTATGCCTGTCTGCGGACGGCCAGTCACCCCCATTGCCCAGAATCAGACGACCC
TCGGTTCTTCCAGAGCCAAGCTGGGCAACTTCCCCTGGCAAGCCTTCACCAGTATCCACGGCCGTGGG
GGCGGGGCCCTGCTGGGGGACAGATGGATCCTCACTGCTGCCCACACCATCTACCCCAAGGACAGTGT
TTCTCTCAGGAAGAACCAGAGTGTGAATGTGTTCTTGGGCCACACAGCCATAGATGAGATGCTGAAAC
TGGGGAACCACCCTGTCCACCGTGTCGTTGTGCACCCCGACTACCGTCAGAATGAGTCCCATAACTTT
AGCGGGGACATCGCCCTCCTGGAGCTGCAGCACAGCATCCCCCTGGGCCCCAACGTCCTCCCGGTCTG
TCTGCCCGATAATGAGACCCTCTACCGCAGCGGCTTGTTGGGCTACGTCAGTGGGTTTGGCATGGAGA
TGGGCTGGCTAACTACTGAGCTGAAGTACTCGAGGCTGCCTGTAGCTCCCAGGGAGGCCTGCAACGCC
TGGCTCCAAAAGAGACAGAGACCCGAGGTGTTTTCTGACAATATGTTCTGTGTTGGGGATGAGACGCA
AAGGCACAGTGTCTGCCAGGGGGACAGTGGCAGCGTCTATGTGGTATGGGACAATCATGCCCATCACT
GGGTGGCCACGGGCATTGTGTCCTGGGGCATAGGGTGTGGCGAAGGGTATGACTTCTACACCAAGGTG
CTCAGCTATGTGGACTGGATCAAAGGGGAGTGATGAATGGCAAGAATAAGCTT

NOV3o, CG102348-06

| | | | |
|---|---|---|---|
| Protein Sequence | SEQ ID NO: 44 | 487 aa | MW at 53528.0kD |

MPGPRVWGKYLWRSPHSKGCPGAMWWLLLWGVLQACPTRGSVLLAQELPQQLTSPGYPEPYGKGQESS
TDIKAPEGFAVRLVFQDFDLEPSQDCAGDSVTISFVGSDPSQFCGQQGSPLGRPPGORFFVSSGRSLR
LTFRTQPSSENKTAHLHKGFLALYQTVAVNYSQPISEASRGSEAINAPGDNPAKVQNHCQEPYYQATA
AGALTCATPGTWKDRQDGEEVLQCMPVCGRPVTPIAQNQTTLGSSRAKLGNFPWQAFTSIHGRGGGAL
LGDRWILTAAHTIYPKDSVSLRKNQSVNVFLGHTAIDEMLKLGNHPVHRVVVHPDYRQNESHNFSGDI
ALLELQHSIPLGPNVLPVCLPDNETLYRSGLLGYVSGFGMEMGWLTTELKYSRLPVAPREACNAWLQK
RQRPEVFSDNMFCVGDETQRHSVCQGDSGSVYVVWDNHAHHWVATGIVSWGIGCGEGYDFYTKVLSYV
DWIKGVMNGKN

| | |
|---|---|
| SEQ ID NO: 45 | 3345 bp |

| | | |
|---|---|---|
| NOV3p, SNP 13376570 of | ORF Start: ATG at 18 | ORF Stop: TGA at 1479 |

| | | |
|---|---|---|
| CG102348-01, DNA Sequence | SNP Pos: 343 | SNP Change: G to A |

CAGATGTCCAGTTCCAGATGCCTGGACCCAGAGTGTGGGGGAAATATCTCTGGAGAAGCCCTCACTCC
AAAGGCTGTCCAGGCGCAATGTGGTGGCTGCTTCTCTGGGGAGTCCTCCAGGCTTGCCCAACCCGGGG
CTCCGTCCTCTTGGCCCAAGAGCTACCCCAGCAGCTGACATCCCCCGGGTACCCAGAGCCGTATGGCA
AAGGCCAAGAGAGCAGCACGGACATCAAGGCTCCAGAGGGCTTTGCTGTGAGGCTCGTCTTCCAGGAC
TTCGACCTGGAGCCGTCCCAGGACTGTGCAGGGGACTCTGTCACAATCTCATTCGTCGGTTCGGATCC
AAACCAGTTCTGTGGTCAGCAAGGCTCCCCTCTGGGCAGGCCCCCTGGTCAGAGGGAGTTTGTATCCT
CAGGGAGGAGTTTGCGGCTGACCTTCCGCACACAGCCTTCCTCGGAGAACAAGACTGCCCACCTTCAC
AAGGGCTTTCTGGCCCTCTACCAAACCGTGGCTGTGAACTATAGTCAGCCCATCAGCGAGGCCAGCAG
GGGCTCTGAGGCCATCAACGCACCTGGAGACAACCCTGCCAAGGTCCAGAACCACTGCCAGGAGCCCT
ATTATCAGGCCGCGGCAGCAGGGGCACTCACCTGTGCAACCCCAGGGACCTGGAAAGACAGACAGGAT
GGGGAGGAGGTTCTTCAGTGTATGCCTGTCTGCGGACGGCCAGTCACCCCCATTGCCCAGAATCAGAC
GACCCTCGGTTCTTCCAGAGCCAAGCTGGGCAACTTCCCCTGGCAAGCCTTCACCAGTATCCACGGCC
GTGGGGGCGGGGCCCTGCTGGGGGACAGATGGATCCTCACTGCTGCCCACACCGTCTACCCCAAGGAC
AGTGTTTCTCTCAGGAAGAACCAGAGTGTGAATGTGTTCTTGGGCCACACAGCCATAGATGAGATGCT
GAAACTGGGGAACCACCCTGTCCACCGTGTCGTTGTGCACCCCGACTACCGTCAGAATGAGTCCCATA
ACTTTAGCGGGGACATCGCCCTCCTGGAGCTGCAGCACAGCATCCCCCTGGGCCCCAACGTCCTCCCG
GTCTGTCTGCCCGATAATGAGACCCTCTACCGCAGCGGCTTGTTGGGCTACGTCAGTGGGTTTGGCAT
GGAGATGGGCTGGCTAACTACTGAGCTGAAGTACTCGAGGCTGCCTGTAGCTCCCAGGGAGGCCTGCA
ACGCCTGGCTCCAAAAGAGACAGAGACCCGAGGTGTTTTCTGACAATATGTTCTGTGTTGGGGATGAG
ACGCAAAGGCACAGTGTCTGCCAGGGGGACAGTGGCAGCGTCTATGTGGTATGGGACAATCATGCCCA
TCACTGGGTGGCCACGGGCATTGTGTCCTGGGGCATAGGGTGTGGCGAAGGGTATGACTTCTACACCA
AGGTGCTCAGCTATGTGGACTGGATCAAGGGAGTGATGAATGGCAAGAATTGACCCTGGGGGCTTGAA
CAGGGACTGACCAGCACAGTGGAGGCCCCAGGCAACAGAGGGCCTGGAGTGAGGACTGAACACTGGGG
TAGGGGTTGGGGGTGGGGGGTTGGGGGAGGCAGGGAAATCCTATTCACATCACTGTTGCACCAAGCCA
CTGCAAGAGAAACCCCCACCCGGCAAGCCCGCCCCATCCCAGACAGGAAGCAGAGTCCCACAGACCGC
TCCTCCTCACCCTCTACCTCCCTGTGCTCATGCACTAGGCCCCGGGAAGCCTGTACATCTCAACAACT
TTCGCCTTGAATGTCCTTAGAACCACCTTCCCCTACTTCATCTGTTGACACAGCTTTTATACTCACCT
GTGGAAGAGTCAGCTACTCACCCGCTATTAGAGTATGGAGGAAGGGGTTTTCATTGCATTGCATTTCT
GAAACATTCCTAAGACCCTTTAGTTGACCTTCAAATATTCAAGCTATTCTGCAGCTCCAAGATGCAAT
TATAGAAACAGCTCCTTTTTTATTTTATGTCCTCTATATGCCAGGTGCTTCACCTGTTATTTCACTTA
ATCCTCATACCATATTTGCAAAGGATGTGTTATTATCTATGTGTGACAAATGAGGAAACTGAGGCTCA
GGGGATAAAGGGACTTGCCCAAGTCCCACAGCTGGTGTGTGACTGCAGAGACTGTGCTCTTCCCAGTG
TGCTGCAATACTTCTCAACCCTCCTCTAACCTGCTGTGTCACCCGCTTTCCCTCCCAGCCCCCACATC
CTTACCATTTTCCCTCCCTGGGAATTCCTGCTTCTGCGAAAATGGTATCCTCTAGCTCACACTTTCCT
AATGGCCCCATCTCCTGCAGAAGCCAGGTGAGCCCAGCACTGGACTGAAGTTCTTGCAGACACCCCAC
CTGTGCCCCTATCATCAGGGGAACTGCTCCACCTGAGAGGACCAACTCTTTAATTTTTAGTAAAACCT
GAAGGTGATGGGCCGGGCGCAGTGGCTCACGCCTGTAATCCCAACACCTTAGGAGTCCGAGGTGGGTG
GATCACGAGGTCAGGAGATCCAGCCCATCCTGGCCAACATGGTGAAACCCCATCTCTACTAAAAATAC
AAAAATTAGCCGGGCGTGGTGACACGTGCCTGTAGTCCCAGCTACTCGGGAGGCTGAGGCAGGAGAAT
CACTTGAACCTGGGAGGCGGAGGTTGCAGTGAGCTAAGATCACGCCACTGCACTCCAGCCTGCGGACA
GACCAAGACTTCATCCCCCCCAAAAAAAAAAGATTGGAGGTGATTTACAGTGAAAGACACAAATAAAA
TACAACTGTTCAATGGAAATAGAAAATAAACACCATAAAAGAGAGAAGAGAGGTAATTTGTTAGCATC
AAGAGTCAAGTTGCTATATGGTCAAAGGTTAAATTTATCTCTAAAAAATGGCAGGATTCAAAGTTGTA
CATACATGTGATTACTTCTGTTTTTTACACCCACATACAGTACAAAAGATTATTAAAAATATTCCCAA
AAGGCAGGTGCAATGATGCACACTTATACCCCCAGCCACTCAGGAGGCTGATGCAAGAGGATCGCTTG
AGCCCAGGAGTTGAAGTCCAGCCTAAGCAACATAGTGAAACCCCATCTCCAAAAATATAATAATAATT
CTCTCAAAATACTAAACAGAGGTGGTTTTATTGATAAGATTTTGGCTGTTTGGTTTTCCACTATTCTC
TATTGGCTAAAATTTGTTTAATGAGCATGAAATGTTTTTATTTTATTTTGCTTATTTTTATGATTGCA
AAAAATGATATGAGTTTCTCCCTGCCAAGGCAAAAAATATATATATATACCTATAAAAAAAAAAAAAA
AAAAAAAAAAA

| | | |
|---|---|---|
| NOV3p, SNP13376570 of | SEQ ID NO: 46 | MW at 53510.9kD |

| | | | |
|---|---|---|---|
| CG102348-01, Protein Sequence | SNP Pos: 109 | 487 aa | SNP Change: Ser to Asn |

MPGPRVWGKYLWRSPHSKGCPGAMWWLLLWGVLQACPTRGSVLLAQELPQQLTSPGYPEPYGKGQESS
TDIKAPEGFAVRLVFQDFDLEPSQDCAGDSVTISFVGSDPNQFCGQQGSPLGRPPGQREFVSSGRSLR
LTFRTQPSSENKTAHLHKGFLALYQTVAVNYSQPISEASRGSEAINAPGDNPAKVQNHCOEPYYQAAA
AGALTCATPGTWKDRQDGEEVLQCMPVCGRPVTPIAQNQTTLGSSRAKLGNFPWQAFTSIHGRGGGAL
LGDRWILTAAHTVYPKDSVSLRKNQSVNVFLGHTAIDEMLKLGNHPVHRVVVHPDYRQNESHNFSGDI
ALLELQHSIPLGPNVLPVCLPDNETLYRSGLLGYVSGFGMEMGWLTTELKYSRLPVAPREACNAWLQK
RQRPEVFSDNMFCVGDETQRHSVCQGDSGSVYVVWDNHAHHWVATGIVSWGIGCGEGYDFYTKVLSYV
DWIKGVMNGKN

| | | |
|---|---|---|
| NOV3q, SNP13376568 of | SEQ ID NO: 47 | 3345 bp |
| CG102348-01, DNA Sequence | ORF Start: ATG at 18 | ORF Stop: TGA at 1479 |

| | |
|---|---|
| SNP Pos: 564 | SNP Change: G to A |

CAGATGTCCAGTTCCAGATGCCTGGACCCAGAGTGTGGGGGAAATATCTCTGGAGAAGCCCTCACTCC
AAAGGCTGTCCAGGCGCAATGTGGTGGCTGCTTCTCTGGGGAGTCCTCCAGGCTTGCCCAACCCGGGG
CTCCGTCCTCTTGGCCCAAGAGCTACCCCAGCAGCTGACATCCCCCGGGTACCCAGAGCCGTATGGCA
AAGGCCAAGAGAGCAGCACGGACATCAAGGCTCCAGAGGGCTTTGCTGTGAGGCTCGTCTTCCAGGAC
TTCGACCTGGAGCCGTCCCAGGACTGTGCAGGGGACTCTGTCACAATCTCATTCGTCGGTTCGGATCC
AAGCCAGTTCTGTGGTCAGCAAGGCTCCCCTCTGGGCAGGCCCCCTGGTCAGAGGGAGTTTGTATCCT
CAGGGAGGAGTITGCGGCTGACCTTCCGCACACAGCCTTCCTCGGAGAACAAGACTGCCCACCTTCAC
AAGGGCTTTCTGGCCCTCTACCAAACCGTGGCTGTGAACTATAGTCAGCCCATCAGCGAGGCCAGCAG
GGGCTCTGAGGCCATCAACACACCTGGAGACAACCCTGCCAAGGTCCAGAACCACTGCCAGGAGCCCT
ATTATCAGGCCGCGGCAGCAGGGGCACTCACCTGTGCAACCCCAGGGACCTGGAAAGACAGACAGGAT
GGGGAGGAGGTTCTTCAGTGTATGCCTGTCTGCGGACGGCCAGTCACCCCCATTGCCCAGAATCAGAC
GACCCTCGGTTCTTCCAGAGCCAAGCTGGGCAACTTCCCCTGGCAAGCCTTCACCAGTATCCACGGCC
GTGGGGGCGGGGCCCTGCTGGGGGACAGATGGATCCTCACTGCTGCCCACACCGTCTACCCCAAGGAC
AGTGTTTCTCTCAGGAAGAACCAGAGTGTGAATGTGTTCTTGGGCCACACAGCCATAGATGAGATGCT
GAAACTGGGGAACCACCCTGTCCACCGTGTCGTTGTGCACCCCGACTACCGTCAGAATGAGTCCCATA
ACTTTAGCGGGGACATCGCCCTCCTGGAGCTGCAGCACAGCATCCCCCTGGGCCCCAACGTCCTCCCG
GTCTGTCTGCCCGATAATGAGACCCTCTACCGCAGCGGCTTGTTGGGCTACGTCAGTGGGTTTGGCAT
GGAGATGGGCTGGCTAACTACTGAGCTGAAGTACTCGAGGCTGCCTGTAGCTCCCAGGGAGGCCTGCA
ACGCCTGGCTCCAAAAGAGACAGAGACCCGAGGTGTTTTCTGACAATATGTTCTGTGTTGGGGATGAG
ACGCAAAGGCACAGTGTCTGCCAGGGGGACAGTGGCAGCGTCTATGTGGTATGGGACAATCATGCCCA
TCACTGGGTGGCCACGGGCATTGTGTCCTGGGGCATAGGGTGTGGCGAAGGGTATGACTTCTACACCA
AGGTGCTCAGCTATGTGGACTGGATCAAGGGAGTGATGAATGGCAAGAATTGACCCTGGGGGCTTGAA
CAGGGACTGACCAGCACAGTGGAGGCCCCAGGCAACAGAGGGCCTGGAGTGAGGACTGAACACTGGGG
TAGGGGTTGGGGGTGGGGGGTTGGGGGAGGCAGGGAAATCCTATTCACATCACTGTTGCACCAAGCCA
CTGCAAGAGAAACCCCCACCCGGCAAGCCCGCCCCATCCCAGACAGGAAGCAGAGTCCCACAGACCGC
TCCTCCTCACCCTCTACCTCCCTGTGCTCATGCACTAGGCCCCGGGAAGCCTGTACATCTCAACAACT
TTCGCCTTGAATGTCCTTAGAACCACCTTCCCCTACTTCATCTGTTGACACAGCTTTTATACTCACCT
GTGGAAGAGTCAGCTACTCACCCGCTATTAGAGTATGGAGGAAGGGGTTTTCATTGCATTGCATTTCT
GAAACATTCCTAAGACCCTTTAGTTGACCTTCAAATATTCAAGCTATTCTGCAGCTCCAAGATGCAAT
TATAGAAACAGCTCCTTTTTTATTTTATGTCCTCTATATGCCAGGTGCTTCACCTGTTATTTCACTTA
ATCCTCATACCATATTTGCAAAGGATGTGTTATTATCTATGTGTGACAAATGAGGAAACTGAGGCTCA
GGGGATAAAGGGACTTGCCCAAGTCCCACAGCTGGTGTGTGACTGCAGAGACTGTGCTCTTCCCAGTG
TGCTGCAATACTTCTCAACCCTCCTCTAACCTGCTGTGTCACCCGCTTTCCCTCCCAGCCCCCACATC
CTTACCATTTTCCCTCCCTGGGAATTCCTGCTTCTGCGAAAATGGTATCCTCTAGCTCACACTTTCCT
AATGGCCCCATCTCCTGCAGAAGCCAGGTGAGCCCAGCACTGGACTGAAGTTCTTGCAGACACCCCAC
CTGTGCCCCTATCATCAGGGGAACTGCTCCACCTGAGAGGACCAACTCTTTAATTTTTAGTAAAACCT
GAAGGTGATGGGCCGGGCGCAGTGGCTCACGCCTGTAATCCCAACACCTTAGGAGTCCGAGGTGGGTG
GATCACGAGGTCAGGAGATCCAGCCCATCCTGGCCAACATGGTGAAACCCCATCTCTACTAAAAATAC
AAAAATTAGCCGGGCGTGGTGACACGTGCCTGTAGTCCCAGCTACTCGGGAGGCTGAGGCAGGAGAAT
CACTTGAACCTGGGAGGCGGAGGTTGCAGTGAGCTAAGATCACGCCACTGCACTCCAGCCTGCGGACA
GACCAAGACTTCATCCCCCCCAAAAAAAAAAGATTGGAGGTGATTTACAGTGAAAGACACAAATAAAA
TACAACTGTTCAATGGAAATAGAAAATAAACACCATAAAAGAGAGAAGAGAGGTAATTTGTTAGCATC
AAGAGTCAAGTTGCTATATGGTCAAAGGTTAAATTTATCTCTAAAAAATGGCAGGATTCAAAGTTGTA
CATACATGTGATTACTTCTGTTTTTTACACCCACATACAGTACAAAAGATTATTAAAAATATTCCCAA
AAGGCAGGTGCAATGATGCACACTTATACCCCCAGCCACTCAGGAGGCTGATGCAAGAGGATCGCTTG
AGCCCAGGAGTTGAAGTCCAGCCTAAGCAACATAGTGAAACCCCATCTCCAAAAATATAATAATAATT
CTCTCAAAATACTAAACAGAGGTGGTTTTATTGATAAGATTTTGGCTGTTTGGTTTTCCACTATTCTC
TATTGGCTAAAATTTGTTTAATGAGCATGAAATGTTTTTATTTTATTTTGCTTATTTTTATGATTGCA
AAAAATGATATGAGTTTCTCCCTGCCAAGGCAAAAAATATATATATATACCTATAAAAAAAAAAAAAA
AAAAAAAAAAAAA

| | | |
|---|---|---|
| NOV3q, SNP13376568 of | SEQ ID NO: 48 | MW at 53513.9kD |

| | | | |
|---|---|---|---|
| CG102348-01, Protein Sequence | SNP Pos: 183 | 487 aa | SNP Change: Ala to Thr |

MPGPRVWGKYLWRSPHSKGCPGAMWWLLLWGVLQACPTRGSVLLAQELPQQLTSPGYPEPYGKGQESS
TDIKAPEGFAVRLVFQDFDLEPSQDCAGDSVTISFVGSDPSQFCGQQGSPLGRPPGQREFVSSGRSLR
LTFRTQPSSENKTAHLHKGFLALYQTVAVNYSQPISEASRGSEAINTPGDNPAKVQNHCQEPYYQAAA
AGALTCATPGTWKDRQDGEEVLQCMPVCGRPVTPIAQNQTTLGSSRAKLGNFPWQAFTSIHGRGGGAL
LGDRWILTAAHTVYPKDSVSLRKNQSVNVFLGHTAIDEMLKLGNHPVHRVVVHPDYRQNESHNFSGDI
ALLELONSIPLGPNVLPVCLPDNETLYRSGLLGYVSGFGMEMGWLTTELKYSRLPVAPREACNAWLQK
RQRPEVFSDNMFCVGDETRQRHSVCQGDSGSVYVVWDNHAHHWVATGISWGIGCGEGYDFYTKVLSYV
DWIKGVNNGKN

| | |
|---|---|
| SEQ ID NO: 49 | 3345 bp |

| | | |
|---|---|---|
| NOV3r, SNP13382463 of | ORF Start: ATG at 18 | ORF Stop: TGA at 1479 |

| | | |
|---|---|---|
| CG102348-01, DNA Sequence | SNP Pos: 693 | SNP Change: C to T |

CAGATGTCCAGTTCCAGATGCCTGGACCCAGAGTGTGGGGGAAATATCTCTGGAGAAGCCCTCACTCC
AAAGGCTGTCCAGGCGCAATGTGGTGGCTGCTTCTCTGGGGAGTCCTCCAGGCTTGCCCAACCCGGGG
CTCCGTCCTCTTGGCCCAAGAGCTACCCCAGCAGCTGACATCCCCCGGGTACCCAGAGCCGTATGGCA
AAGGCCAAGAGAGCAGCACGGACATCAAGGCTCCAGAGGGCTTTGCTGTGAGGCTCGTCTTCCAGGAC
TTCGACCTGGAGCCGTCCCAGGACTGTGCAGGGGACTCTGTCACAATCTCATTCGTCGGTTCGGATCC
AAGCCAGTTCTGTGGTCAGCAAGGCTCCCCTCTGGGCAGGCCCCCTGGTCAGAGGGAGTTTGTATCCT
CAGGGAGGAGTTTGCGGCTGACCTTCCGCACACAGCCTTCCTCGGAGAACAAGACTGCCCACCTTCAC
AAGGGCTTTCTGGCCCTCTACCAAACCGTGGCTGTGAACTATAGTCAGCCCATCAGCGAGGCCAGCAG
GGGCTCTGAGGCCATCAACGCACCTGGAGACAACCCTGCCAAGGTCCAGAACCACTGCCAGGAGCCCT
ATTATCAGGCCGCGGCAGCAGGGGCACTCACCTGTGCAACCCCAGGGACCTGGAAAGACAGACAGGAT
GGGGAGGAGGTTTTTCAGTGTATGCCTGTCTGCGGACGGCCAGTCACCCCCATTGCCCAGAATCAGAC
GACCCTCGGTTCTTCCAGAGCCAAGCTGGGCAACTTCCCCTGGCAAGCCTTCACCAGTATCCACGGCC
GTGGGGGCGGGGCCCTGCTGGGGGACAGATGGATCCTCACTGCTGCCCACACCGTCTACCCCAAGGAC
AGTGTTTCTCTCAGGAAGAACCAGAGTGTGAATGTGTTCTTGGGCCACACAGCCATAGATGAGATGCT
GAAACTGGGGAACCACCCTGTCCACCGTGTCGTTGTGCACCCCGACTACCGTCAGAATGAGTCCCATA
ACTTTAGCGGGGACATCGCCCTCCTGGAGCTGCAGCACAGCATCCCCCTGGGCCCCAACGTCCTCCCG
GTCTGTCTGCCCGATAATGAGACCCTCTACCGCAGCGGCTTGTTGGGCTACGTCAGTGGGTTTGGCAT
GGAGATGGGCTGGCTAACTACTGAGCTGAAGTACTCGAGGCTGCCTGTAGCTCCCAGGGAGGCCTGCA
ACGCCTGGCTCCAAAAGAGACAGAGACCCGAGGTGTTTTCTGACAATATGTTCTGTGTTGGGGATGAG
ACGCAAAGGCACAGTGTCTGCCAGGGGGACAGTGGCAGCGTCTATGTGGTATGGGACAATCATGCCCA
TCACTGGGTGGCCACGGGCATTGTGTCCTGGGGCATAGGGTGTGGCGAAGGGTATGACTTCTACACCA
AGGTGCTCAGCTATGTGGACTGGATCAAGGGAGTGATGAATGGCAAGAATTGACCCTGGGGGCTTGAA
CAGGGACTGACCAGCACAGTGGAGGCCCCAGGCAACAGAGGGCCTGGAGTGAGGACTGAACACTGGGG
TAGGGGTTGGGGGTGGGGGGTTGGGGGAGGCAGGGAAATCCTATTCACATCACTGTTGCACCAAGCCA
CTGCAAGAGAAACCCCCACCCGGCAAGCCCGCCCCATCCCAGACAGGAAGCAGAGTCCCACAGACCGC
TCCTCCTCACCCTCTACCTCCCTGTGCTCATGCACTAGGCCCCGGGAAGCCTGTACATCTCAACAACT
TTCGCCTTGAATGTCCTTAGAACCACCTTCCCCTACTTCATCTGTTGACACAGCTTTTATACTCACCT
GTGGAAGAGTCAGCTACTCACCCGCTATTAGAGTATGGAGGAAGGGGTTTTCATTGCATTGCATTTCT
GAAACATTCCTAAGACCCTTTAGTTGACCTTCAAATATTCAAGCTATTCTGCAGCTCCAAGATGCAAT
TATAGAAACAGCTCCTTTTTTATTTTATGTCCTCTATATGCCAGGTGCTTCACCTGTTATTTCACTTA
ATCCTCATACCATATTTGCAAAGGATGTGTTATTATCTATGTGTGACAAATGAGGAAACTGAGGCTCA
GGGGATAAAGGGACTTGCCCAAGTCCCACAGCTGGTGTGTGACTGCAGAGACTGTGCTCTTCCCAGTG
TGCTGCAATACTTCTCAACCCTCCTCTAACCTGCTGTGTCACCCGCTTTCCCTCCCAGCCCCCACATC
CTTACCATTTTCCCTCCCTGGGAATTCCTGCTTCTGCGAAAATGGTATCCTCTAGCTCACACTTTCCT
AATGGCCCCATCTCCTGCAGAAGCCAGGTGAGCCCAGCACTGGACTGAAGTTCTTGCAGACACCCCAC
CTGTGCCCCTATCATCAGGGGAACTGCTCCACCTGAGAGGACCAACTCTTTAATTTTTAGTAAAACCT
GAAGGTGATGGGCCGGGCGCAGTGGCTCACGCCTGTAATCCCAACACCTTAGGAGTCCGAGGTGGGTG
GATCACGAGGTCAGGAGATCCAGCCCATCCTGGCCAACATGGTGAAACCCCATCTCTACTAAAAATAC
AAAAATTAGCCGGGCGTGGTGACACGTGCCTGTAGTCCCAGCTACTCGGGAGGCTGAGGCAGGAGAAT
CACTTGAACCTGGGAGGCGGAGGTTGCAGTGAGCTAAGATCACGCCACTGCACTCCAGCCTGCGGACA
GACCAAGACTTCATCCCCCCCAAAAAAAAAAGATTGGAGGTGATTTACAGTGAAAGACACAAATAAAA
TACAACTGTTCAATGGAAATAGAAAATAAACACCATAAAAGAGAGAAGAGAGGTAATTTGTTAGCATC
AAGAGTCAAGTTGCTATATGGTCAAAGGTTAAATTTATCTCTAAAAAATGGCAGGATTCAAAGTTGTA
CATACATGTGATTACTTCTGTTTTTTACACCCACATACAGTACAAAAGATTATTAAAAATATTCCCAA
AAGGCAGGTGCAATGATGCACACTTATACCCCCAGCCACTCAGGAGGCTGATGCAAGAGGATCGCTTG
AGCCCAGGAGTTGAAGTCCAGCCTAAGCAACATAGTGAAACCCCATCTCCAAAAATATAATAATAATT
CTCTCAAAATACTAAACAGAGGTGGTTTTATTGATAAGATTTTGGCTGTTTGGTTTTCCACTATTCTC
TATTGGCTAAAATTTGTTTAATGAGCATGAAATGTTTTTATTTTATTTTGCTTATTTTTATGATTGCA
AAAAATGATATGAGTTTCTCCCTGCCAAGGCAAAAAATATATATATATACCTATAAAAAAAAAAAAAA
AAAAAAAAAAAAA

| | | |
|---|---|---|
| NOV3r, SNP13382463 of | SEQ ID NO: 50 | MW at 53517.9kD |

| | | | |
|---|---|---|---|
| CG102348-01, Protein Sequence | SNP Pos: 226 | 487 aa | SNP Change: Leu to Phe |

MPGPRVWGKYLWRSPNSKGCPGAMWWLLLWGVLQACPTRGSVLLAQELPQQLTSPGYPEPYGKGQESS
TDIKAPEGFAVRLVFQDFDLEPSQDCAGDSVTISFVGSDPSQFCGQQGSPLGRPPGQREFVSSGRSLR
LTFRTQPSSENKTAHLHKGFLALYQTVAVNYSQPISEASRGSEAINAPGDNPAKVQNHCQEPYYQAAA
AGALTCATPGTWKDRQDGEEVFQCMPVCGRPVTPIAQNQTTLGSSRAKLGNFPWQAFTSIHGRGGGAL
LGDRWILTAAHTVYPKDSVSLRKNQSVNVFLGHTAIDEMLKLGNHPVHRVVVHPDYRQNESHNFSGDI
ALLELQHSIPLGPNVLPVCLPDNETLYRSGLLGYVSGFGMEMGWLTTELKYSRLPVAPREACNAWLQK
RQRPEVFSDNNFCVGDETQRHSVCQGDSGSVYVVWDNHAHHWVATGIVSWGIGCGEGYDFYTKVLSYV
DWIKGVMNGKN

| | |
|---|---|
| SEQ ID NO: 51 | 3345 bp |

| | | |
|---|---|---|
| NOV3s, SNP13374245 of | ORF Start: ATG at 18 | ORF Stop: TGA at 1479 |

| | | |
|---|---|---|
| CG102348-01, DNA Sequence | SNP Pos: 1459 | SNP Change: G to A |

CAGATGTCCAGTTCCAGATGCCTGGACCCAGAGTGTGGGGGAAATATCTCTGGAGAAGCCCTCACTCC
AAAGGCTGTCCAGGCGCAATGTGGTGGCTGCTTCTCTGGGGAGTCCTCCAGGCTTGCCCAACCCGGGG
CTCCGTCCTCTTGGCCCAAGAGCTACCCCAGCAGCTGACATCCCCCGGGTACCCAGAGCCGTATGGCA
AAGGCCAAGAGAGCAGCACGGACATCAAGGCTCCAGAGGGCTTTGCTGTGAGGCTCGTCTTCCAGGAC
TTCGACCTGGAGCCGTCCCAGGACTGTGCAGGGGACTCTGTCACAATCTCATTCGTCGGTTCGGATCC
AAGCCAGTTCTGTGGTCAGCAAGGCTCCCCTCTGGGCAGGCCCCCTGGTCAGAGGGAGTTTGTATCCT
CAGGGAGGAGTTTGCGGCTGACCTTCCGCACACAGCCTTCCTCGGAGAACAAGACTGCCCACCTTCAC
AAGGGCTTTCTGGCCCTCTACCAAACCGTGGCTGTGAACTATAGTCAGCCCATCAGCGAGGCCAGCAG
GGGCTCTGAGGCCATCAACGCACCTGGAGACAACCCTGCCAAGGTCCAGAACCACTGCCAGGAGCCCT
ATTATCAGGCCGCGGCAGCAGGGGCACTCACCTGTGCAACCCCAGGGACCTGGAAAGACAGACAGGAT
GGGGAGGAGGTTCTTCAGTGTATGCCTGTCTGCGGACGGCCAGTCACCCCCATTGCCCAGAATCAGAC
GACCCTCGGTTCTTCCAGAGCCAAGCTGGGCAACTTCCCCTGGCAAGCCTTCACCAGTATCCACGGCC
GTGGGGGCGGGGCCCTGCTGGGGGACAGATGGATCCTCACTGCTGCCCACACCGTCTACCCCAAGGAC
AGTGTTTCTCTCAGGAAGAACCAGAGTGTGAATGTGTTCTTGGGCCACACAGCCATAGATGAGATGCT
GAAACTGGGGAACCACCCTGTCCACCGTGTCGTTGTGCACCCCGACTACCGTCAGAATGAGTCCCATA
ACTTTAGCGGGGACATCGCCCTCCTGGAGCTGCAGCACAGCATCCCCCTGGGCCCCAACGTCCTCCCG
GTCTGTCTGCCCGATAATGAGACCCTCTACCGCAGCGGCTTGTTGGGCTACGTCAGTGGGTTTGGCAT
GGAGATGGGCTGGCTAACTACTGAGCTGAAGTACTCGAGGCTGCCTGTAGCTCCCAGGGAGGCCTGCA
ACGCCTGGCTCCAAAAGAGACAGAGACCCGAGGTGTTTTCTGACAATATGTTCTGTGTTGGGGATGAG
ACGCAAAGGCACAGTGTCTGCCAGGGGGACAGTGGCAGCGTCTATGTGGTATGGGACAATCATGCCCA
TCACTGGGTGGCCACGGGCATTGTGTCCTGGGGCATAGGGTGTGGCGAAGGGTATGACTTCTACACCA
AGGTGCTCAGCTATGTGGACTGGATCAAGGAAGTGATGAATGGCAAGAATTGACCCTGGGGGCTTGAA
CAGGGACTGACCAGCACAGTGGAGGCCCCAGGCAACAGAGGGCCTGGAGTGAGGACTGAACACTGGGG
TAGGGGTTGGGGGTGGGGGGTTGGGGGAGGCAGGGAAATCCTATTCACATCACTGTTGCACCAAGCCA
CTGCAAGAGAAACCCCCACCCGGCAAGCCCGCCCCATCCCAGACAGGAAGCAGAGTCCCACAGACCGC
TCCTCCTCACCCTCTACCTCCCTGTGCTCATGCACTAGGCCCCGGGAAGCCTGTACATCTCAACAACT
TTCGCCTTGAATGTCCTTAGAACCACCTTCCCCTACTTCATCTGTTGACACAGCTTTTATACTCACCT
GTGGAAGAGTCAGTTACTCACCCGCTATTAGAGTATGGAGGAAGGGGTTTTCATTGCATTGCATTTCT
GAAACATTCCTAAGACCCTTTAGTTGACCTTCAAATATTCAAGCTATTCTGCAGCTCCAAGATGCAAT
TATAGAAACAGCTCCTTTTTTATTTTATGTCCTCTATATGCCAGGTGCTTCACCTGTTATTTCACTTA
ATCCTCATACCATATTTGCAAAGGATGTGTTATTATCTATGTGTGACAAATGAGGAAACTGAGGCTCA
GGGGATAAAGGGACTTGCCCAAGTCCCACAGCTGGTGTGTGACTGCAGAGACTGTGCTCTTCCCAGTG
TGCTGCAATACTTCTCAACCCTCCTCTAACCTGCTGTGTCACCCGCTTTCCCTCCCAGCCCCCACATC
CTTACCATTTTCCCTCCCTGGGAATTCCTGCTTCTGCGAAAATGGTATCCTCTAGCTCACACTTTCCT
AATGGCCCCATCTCCTGCAGAAGCCAGGTGAGCCCAGCACTGGACTGAAGTTCTTGCAGACACCCCAC
CTGTGCCCCTATCATCAGGGGAACTGCTCCACCTGAGAGGACCAACTCTTTAATTTTTAGTAAAACCT
GAAGGTGATGGGCCGGGCGCAGTGGCTCACGCCTGTAATCCCAACACCTTAGGAGTCCGAGGTGGGTG
GATCACGAGGTCAGGAGATCCAGCCCATCCTGGCCAACATGGTGAAACCCCATCTCTACTAAAAATAC
AAAATTAGCCGGGCGTGGTGACACGTGCCTGTAGTCCCAGCTACTCGGGAAGGCTGAGGCAGGAGAAT
CACTTGAACCTGGGAGGCGGAGGTTGCAGTGAGCTAAGATCACGCCACTGCACTCCAGCCTGCGGACA
GACCAAGACTTCATCCCCCCCAAAAAAAAAAGATTGGAGGTGATTTACAGTGAAAGACACAAATAAAA
TACAACTGTTCAATGGAAATAGAAAATAAACACCATAAAAGAGAGAAGAGAGGTAATTTGTTAGCATC
AAGAGTCAAGTTGCTATATGGTCAAAGGTTAAATTTATCTCTAAAAAATGGCAGGATTCAAAGTTGTA
CATACATGTGATTACTTCTGTTTTTTACACCCACATACAGTACAAAAGATTATTAAAAATATTCCCAA
AAGGCAGGTGCAATGATGCACACTTATACCCCCAGCCACTCAGGAGGCTGATGCAAGAGGATCGCTTG
AGCCCAGGAGTTGAAGTCCAGCCTAAGCAACATAGTGAAACCCCATCTCCAAAAATATAATAATAATT
CTCTCAAAATACTAAACAGAGGTGGTTTTATTGATAAGATTTTGGCTGTTTGGTTTTCCACTATTCTC
TATTGGCTAAAATTTGTTTAATGAGCATGAAATGTTTTTATTTTATTTTGCTTATTTTTATGATTGCA
AAAAATGATATGAGTTTCTCCCTGCCAAGGCAAAAAATATATATATATACCTATAAAAAAAAAAAAAA
AAAAAAAAAAAAA

| | | |
|---|---|---|
| NOV3s, SNP13374245 of | SEQ ID NO: 52 | MW at 53556.0kD |

| | | | |
|---|---|---|---|
| CG102348-01, Protein Sequence | SNP Pos: 481 | 487 aa | SNP Change: Gly to Glu |

MPGPRVWGKYLWRSPHSKGCPGAMWLLWGVLQACPTRGSVLLAQELPQQLTSPGYPEPYGKGQESS
TDIKAPEGFAVRLVFQDFDLEPSQDCAGDSVTISFVGSDPSQFCGQQGSPLGRPPGQREFVSSGRSLR
LTFRTQPSSENKTAHLHKGFLALYQTVAVNYSQPISEASRGSEAINAPGDNPAKVQNHCQEPYYQAAA
AGALTCATPGTWKDRQDGEEVLQCMPVCGRPVTPIAQNQTTLGSSRAKLGNFPWQAFTSIHGRGGGAL
LGDRWILTAAHTVYPKDSVSLRKNQSVNVFLGHTAIDEMLKLGNHPVHRVVVHPDYRQNESHNFSGDI
ALLELQHSIPLGPNVLPVCLPDNETLYRSGLLGYVSGFGMEMGWLTTELKYSRLPVAPREACNAWLQK
RQRPEVFSDNMFCVGDETQRHSVCQGDSGSVYVVWDNHAHHWVATGIVSWGIGCGEGYDFYTKVLSYV
DWIKEVMNGKN

A ClustalW comparison of the above protein sequences yields the following sequence alignment shown in Table 3B.

TABLE 3B
Comparison of the NOV3 protein sequences.

| | |
|---|---|
| NOV3a | ----MPGPRVWGKYLWRSPHSKGCPGAMWWLLLWGVLQACPTRGSVLLAQELPQQLTSPG |
| NOV3b | -------------------------------------KLCPTRGSVLLAQELPQQLTSPG |
| NOV3c | ------------------------------------------------------------ |
| NOV3d | -------------------------------------KLCPTRGSVLLAQELPQQLTSPG |
| NOV3e | -------------------------------------KLCPTRGSVLLAQELPQQLTSPG |
| NOV3f | KLPTMPGPRVWGKYLWRSPHSKGCPGAMWWLLLWGVLQACPTRGSVLLAQELPQQLTSPG |
| NOV3g | -------------------------------------KLCPTRGSVLLAQELPQQLTSPG |
| NOV3h | -------------------------------------KLCPTRGSVLLAQELPQQLTSPG |
| NOV3i | -------------------------------------KLCPTRGSVLLAQELPQQLTSPG |
| NOV3j | -------------------------------------KLCPTRGSVLLAQELPQQLTSPG |
| NOV3k | ---------------------------------------CPTRGSVLLAQELPQQLTSPG |
| Nov3l | ------------------------------------------------------------ |
| NOV3m | ---------------------------------------CPTRGSVLLAQELPQQLTSPG |
| NOV3n | ---------------------------------------CPTRGSVLLAQELPQQLTSPG |
| NOV3o | ----MPGPRVWGKYLWRSPHSKGCPGAMWWLLLWGVLQACPTRGSVLLAQELPQQLTSPG |
| NOV3a | YPEPYGKGQESSTDIKAPEGFAVRLVFQDFDLEPSQDCAGDSVTISFVGSDPSQFCGQQG |
| NOV3b | YPEPYGKGQESSTDIKAPEGFAVRFVFQDFDLESSQDCAGDSVTISFVGSDPSQFCGQQG |
| NOV3c | ------------------------------------------------------------ |
| NOV3d | YPEPYGKGQESSTDIKAPEGFAVRLVFQDFDLEPSQDCAGDSVTISFVGSDPSQFCGQQG |
| NOV3e | YPEPYGKGQESSTDIKAPEGFAVRLVFQDFDLEPSQDCAGDSVTISFVGSDPSQFCGQQG |
| NOV3f | YPEPYGKGQESSTDIKAPEGFAVRLVFQDFDLEPSQDCAGDSVTVSFVGSDPSQFCGQQG |
| NOV3g | YPEPYGKGQESSTDIKAPEGFAVRLVFQDFDLEPSQDCAGDSVTISFVGSDPSQFCGQQG |
| NOV3h | YPEPYGKGQESSTDIKAPEGFAVRLVFQDFDLEPSQDCAGDSVTISFVGSDPSQFCGQQG |
| NOV3i | YPEPYGKGQESSTDIKAPEGFAVRLVFQDFDLEPSQDCAGDSVTISFVGSDPSQFCGQQG |
| NOV3j | YPEPYGKGQESSTDIKAPEGFAVRLVFQDFDLEPSQDCAGDSVTISFVGSDPSQFCGQQG |
| NOV3k | YPEPYGKGQESSTDIKAPEGFAVRLVFQDFDLEPSQDCAGDSVTISFVGSDPSQFCGQQG |
| NOV3l | ------------------------------------------------------------ |
| NOV3m | YPEPYGKGQESSTDIKAPEGFAVRLVFQDFDLEPSQDCAGDSVTISFVGSDPSQFCGQQG |
| NOV3n | YPEPYGKGQESSTDIKAPEGFAVRFVFQDFDLESSQDCAGDSVTISFVGSDPSQFCGQQG |
| NOV3o | YPEPYGKGQESSTDIKAPEGFAVRFVFQDFDLESSQDCAGDSVTISFVGSDPSQFCGQQG |
| NOV3a | SPLGRPPGQREFVSSGRSLRLTFRTQPSSENKTAHLHKGFLALYQTVAVNYSQPISEASR |
| NOV3b | SPLGRPPGQREFVSSGRSLRLTFRTQPSSENKTAHLHKGFLALYQTVAVNYSQPISEASR |
| NOV3c | ------------------------------------------------------------ |
| NOV3d | SPLGRPPGQREFVSSGRSLRLTFRTQPSSENKTAHLHKGFLALYQTVAVNYSQPISEASR |
| NOV3e | SPLGRPPGQREFVSSGRSLRLTFRTQPSSEDKTAHLHKGFLALYQTVAVNYSQPISEASR |
| NOV3f | SPLGRPPGQREFVSSGRSLRLTFRTQPSSENKTAHLHKGFLALYQTVAVNYSQPISEASR |
| NOV3g | SPLGRPPGQREFVSSGRSLRLTFRTQPSSENKTAHLHKGFLALYQTVAVNYSQPISEASR |
| NOV3h | SPLGRPPGQREFVSSGRSLRLTFRTQP5SENKTAHLHKGFLALYQTVAVNYSQPISEASR |
| NOV3i | SPLGRPPGQREFVSSGRSLRLTFRTQPSSENKTAHLHKGFLALYQTVAVNYSQPISEASR |
| NOV3j | SPLGRPPGQREFVSSGRSLRLTFRTQPSSENKTAHLHKGFLALYKL-------------- |
| NOV3k | SPLGRPPGQREFVSSGRSLRLTFRTQPSSENKTAHLHKGFLALY---------------- |
| NOV3l | ------------------------------------------------------------ |
| NOV3m | SPLGRPPGQREFVSSGRSLRLTFRTQPSSENKTAHLHKGFLALYQTVAVNYSQPISEASR |
| NOV3n | SPLGRPPGQREFVSSGRSLRLTFRTQPSSENKTAELHKGFLALYQTVAVNYSQPISEASR |
| NOV3o | SPLGRPPGQREFVSSGRSLRLTFRTQPSSENKTAHLHKGFLALYQTVAVNYSQPISEASR |
| NOV3a | GSEAINAPGDNPAKVQNHCQEPYYQAAAAGALTCATPGTWKDRQDGEEVLQCMPVCGRPV |
| NOV3b | GSEAINAPGDNPAKVQNHCQEPYYQATAAGALTCATPGTWKDRQDGEEVLQCMPVCGRPV |
| NOV3c | ------------------------------------------------------------ |
| NOV3d | GSEAINAPGDNPAKVQNHCQEPYYQAAAAGALTCATPGTWKDRQDGEEVLQCMPVCGRPV |
| NOV3e | GSEAINAPGDNPAKVQNHCQEPYYQAAAAGALTCATPGTWKDRQDGEEVLQCMPVCGRPV |
| NOV3f | GSEAINAPGDNPAKVQNHCQEPYYQAAAAGALTCATPGTWKDRQDGEEVLQCMPVCGRPV |
| NOV3g | GSEAINAPGDNPAKVQNHCQEPYYQAAAA-----------------------V--CGRPV |
| NOV3h | GSEAINAPGDNPAKVQNHCQEPYYQAAAAGALTCATPGTWEDRQDGEEVLQCMPVCGRPV |
| NOV3i | GSEAINAPGDNPAKVQNHCQEPYYQAAAAGALTCATPGTWKDRQDGEEVLQCMPVCGRPV |
| NOV3j | ------------------------------------------------------------ |
| NOV3k | ------------------------------------------------------------ |
| NOV3l | ------------------------------------------------------------ |
| NOV3m | GSEAINAPGDNPAKVQNHCQEPYYQAAAA-----------------------V--CGRPV |
| NOV3n | GSEAINAPGDNPAKVQNHCQEPYYQATAAGALTCATPGTWKDRQDGEEVLQCMPVCGRPV |
| NOV3o | GSEAINAPGDNPAKVQNHCQEPYYQATAAGALTCATPGTWKDRQDGEEVLQCMPVCGRPV |
| NOV3a | TPIAQNQTTLGSSPAKLGNFPWQAFTSIHGRGGGALLGDRWILTAAHTVYPKDSVSLRKN |
| NOV3b | TPIAQNQTTLGSSRAKLGNFPWQAFTSIHGRGGGALLGDRWILTAAHTIYPKDSVSLRKN |
| NOV3c | ------KLTLGSSRAKLGNFPWQAFTSIHGRGGGALLGDRWILTAAHTIYPKNSVSLRKN |
| NOV3d | TPIAQNQTTLGSSRAKLGNFPWQAFTSIHGRGGGALLGDRWILTAAHTVYPKDSVSLRKN |
| NOV3e | TPIAQNQTTLGSSRAKLGNFPWQAFTSIHGRGGGALLGDRWILTAAHTIYPKDSVSLRKN |
| NOV3f | TPIAQNQTTLGSSRAKLGNFPWQAFTSIHGRGGGALLGDRWILTAAHTIYPKDSVSLRKN |
| NOV3g | TPIAQNQTTLGSSRAKLGNFPWQAFTSIHGRGGGALLGDRWILTAAHTIYPKDSVSLRKN |
| NOV3h | TPIAQNQTTLGSSRAKLGNFPWQAFTSIHGRGGGALLGDRWILTAAETIYPKDSVSLRKN |
| NOV3i | TPIAQNQTTLGSSRAKLGNFPWOAFTSIHGRGGGALLGDRWILTAAHTIYPKDSVSLRKN |
| NOV3j | ------------------------------------------------------------ |
| NOV3k | ------------------------------------------------------------ |
| NOV3l | --------TLGSSRAKLGNFPWQAFTSIHGRGGGALLGDRWILTAAHTIYPKNSVSLRKN |
| NOV3m | TPIAQNQTTLGSSRAKLGNFPWQAFTSIHGRGGGALLGDRWILTAAHTIYPKDSVSLRKN |
| NOV3n | TPIAQNQTTLGSSRAKLGNFPWQAFTSIHGRGGGALLGDRWILTAAHTIYPKDSVSLRKN |
| NOV3o | TPIAQNQTTLGSSRAKLGNFPWQAFTSIHGRGGGALLGDRWILTAAHTIYPKDSVSLRKN |
| NOV3a | QSVNVFLGHTAIDEMLKLGNHPVHRVVVHPDYRQNESHNFSGDIALLELQHSIPLGPNVL |
| NOV3b | QSVNVFLGHTAIDEMLKLGNHPVHRVVVHPDYRQNESHNFSGDIALLELQHSIPIGPNVL |
| NOV3c | QSVNVFLGHTAIDEMLKLGNHPVHRVVVHPDYRQNESHNFSGDIALLELQHSIPLGPNVL |
| NOV3d | QSVNVFLGHTAIDEMLKLGNHPVHRVVVHPDYRQNESHNFSGDIALLELQHSIPLGPNVL |
| NOV3e | QSVNVFLGHTAIDEMLKLGNHPVHRVVVHPDYRQNESHNFSGDIALLELQHSIPLGPNVL |
| NOV3f | QSVNVFLGHTAIDEMLKLGNHPVHRVVVHPDYRQNESHNFSGDIALLELQHSIPLGPNVL |
| NOV3g | QSVNVFLGHTAIDEMLKLGNHPVHRVVVHPDYRQNESHNFSGDIALLELQHSIPLGPNVL |
| NOV3h | QSVNVFLGHTAIDEMLKLGNHPVHRVVVHPDYRQNESHNFSGDIALLELQHSIPLGPNVL |
| NOV3i | QSVNVFLGHTAIDEMLKLGNHPVHRVVVHPDYRQNESHNFSGDIALLELRHSIPLGPNVL |
| NOV3j | ------------------------------------------------------------ |
| NOV3k | ------------------------------------------------------------ |
| NOV3l | QSVNVFLGHTAIDEMLKLGNHPVHRVVVHPDYRQNESHNFSGDIALLELQHSIPLGPNVL |
| NOV3m | QSVNVFLGHTAIDEMLKLGNHPVHRVVVHPDYRQNESHNFSGDIALLELQHSIPLGPNVL |
| NOV3n | QSVNVFLGHTAIDEMLKLGNHPVHRVVVHPDYRQNESHNFSGDIALLELQHSIPLGPNVL |
| NOV3o | QSVNVFLGHTAIDEMLKLGNHPVHRVVVHPDYRQNESHNFSGDIALLELQHSIPLGPNVL |
| NOV3a | PVCLPDNETLYRSGLLGYVSGFGMEMGWLTTELKYSRLPVAPREACNAWLQKRQRPEVFS |
| NOV3b | PVCLPDNETLYRSGLLGYVSGFGMEMGWLTTELKYSRLPVAPREACNAWLQKRQRPEVFS |
| NOV3c | PVCLPDNETLYRSGLLGYVSGFGMEMGWLTTELKYSRLPVAPREACNAWLQKRQRPEVFS |
| NOV3d | PVCLPDNETLYRSGLLGYVSGFGMEMGWLTTELKYSRLPVAPREACNAWLQKRQRPEVFS |
| NOV3e | PVCLPDNETLYRSGLLGYVSGFGMEMGWLTTELKYSRLPVAPREAcNAWLQKRQRPEVFS |
| NOV3f | PVCLPDNETLYRSGLLGYVSGFGMEMGWLTTELKYSRLPVAPREACNAWLQKRQRPEVFS |
| NOV3g | PVCLPDNETLYRSGLLGYVSGFGMEMGRLTTELKYSRLPVAPREACNAWLQKRQRPEVFS |
| NOV3h | PVCLPDNETLYRSGLLGYVSGFGMEMGWLTTELKYSRLPVAPREACNAWLQKRQRPEVFS |
| NOV3i | PVCLPDNETLYRSGLLGYVSGFGMEMGWLTTELKYSRLPVAPREACNAWLQKRQRPEVFS |
| NOV3j | ------------------------------------------------------------ |
| NOV3k | ------------------------------------------------------------ |
| NOV3l | PVCLPDNETLYRSGLLGYVSGFGMEMGWLTTELKYSRLPVAPREACNAWLQKRQRPEVFS |
| NOV3m | PVCLPDNETLYRSGLLGYVSGFGMEMGRLTTELKYSRLPVAPREACNAWLQKRQRPEVFS |
| NOV3n | PVCLPDNETLYRSGLLGYVSGFGMEMGWLTTELKYSRLPVAPREACNAWLQKRQRPEVFS |
| NOV3o | PVCLPDNETLYRSGLLGYVSGFGMEMGWLTTELKYSRLPVAPREACNAWLQKRQRPEVFS |
| NOV3a | DNMFCVGDETQRHSVCQODSGSVYVVWDNHAHHWVATGIVSWGIGCGEGYDFYTKVLSYV |
| NOV3b | DNMFCVGDETQRHSVCQGDSGSVYVVWDNHAHHWVATGIVSWGIGCGEGYDFYTKVLSYV |
| NOV3c | DNMFCVGDETQRHSVCQGDSGSVYVVWDNRAHHWVATGIVSWGIGCGEGYDFYTKVLSYV |
| NOV3d | DNMFCVGDETQRHSVCQGDSGSVYVVWDNHAHHWVATGIVSWGIGCGEGYDFYTKVLSYV |
| NOV3e | DNMFCVGDETQRHSVCQGDSGSVYVVWDNHAHHWVATGIVSWGIGCGEGYDFYTKVLSYV |
| NOV3f | DNMFCVGDETQRHSVCQGDSGSVYVVWDNHAHHWVATGIVSWGIGCGEGYDFYTKVLSYV |
| NOV3g | DNMFCVGDETQRHSVCQGDSGSVYVVWDNHAHHWVATGIVSWGIGCGEGYDFYTKVLSYV |
| NOV3h | DNMFCVGDETQRHSVCQGDSGSVYVVWDNHAHHWVATGIVSWGIGCGEGYDFYTKVLSYV |
| NOV3i | DNMFCVGDGTQRHSVCQGDSGSVYVVWDNHAHHWVATGIVSWGIGCGEGYDFYTKVLSYV |
| NOV3j | ------------------------------------------------------------ |
| NOV3k | ------------------------------------------------------------ |
| NOV3l | DNMFCVGDETQRHSVCQGDSGSVYVVWDNRAHHWVATGIVSWGIGCGEGYDFYTKVLSYV |
| NOV3m | DNMFCVGDETQRHSVCQGDSGSVYVVWDNRAHHWVATGIVSWGIGCGEGYDFYTKVLSYV |
| NOV3n | DNMFCVGDETQRHSVCQGDSGSVYVVWDNRAHHWVATGIVSWGIGCGEGYDFYTKVLSYV |
| NOV3o | DNMFCVGDETQRHSVCQGDSGSVYVVWDNRAHHWVATGIVSWGIGCGEGYDFYTKVLSYV |
| NOV3a | DWIKGVMNGKN-- |
| NOV3b | DWIKGVMNGKNKL |
| NOV3c | DWIKGVMNGKNKL |
| NOV3d | DWIKGVMNGKNKL |
| NOV3e | DWIKGVMNGKNKL |
| NOV3f | DWIKGVMNGKNKL |
| NOV3g | DWIKGVMNGKNKL |
| NOV3h | DWIKGVMNGKNKL |
| NOV3i | DWIKGVMNGKNKL |
| NOV3j | ------------- |
| NOV3k | ------------- |
| NOV3l | DWIKGVMNGKN-- |
| NOV3m | DWIKGVMNGKN-- |
| NOV3n | DWIKGVNNGKN-- |
| NOV3o | DWIKGVMNGKN-- |
| NOV3a | (SEQ ID NO: 16) |
| NOV3b | (SEQ ID NO: 18) |
| NOV3c | (SEQ ID NO: 20) |
| NOV3d | (SEQ ID NO: 22) |
| NOV3e | (SEQ ID NO: 24) |
| NOV3f | (SEQ ID NO: 26) |
| NOV3g | (SEQ ID NO: 28) |
| NOV3h | (SEQ ID NO: 30) |
| NOV3i | (SEQ ID NO: 32) |
| NOV3j | (SEQ ID NO: 34) |
| NOV3k | (SEQ ID NO: 36) |
| NOV3l | (SEQ ID NO: 38) |
| NOV3m | (SEQ ID NO: 40) |
| NOV3n | (SEQ ID NO: 42) |
| NOV3o | (SEQ ID NO: 44) |

Further analysis of the NOV3a protein yielded the following properties shown in Table 3C.

TABLE 3C
Protein Sequence Properties NOV3a

| | |
|---|---|
| SignalP analysis: | Cleavage site between residues 36 and 37 |
| PSORT II analysis: | |

| | |
|---|---|
| PSG: | a new signal peptide prediction method |
| | N-region: length 9; pos.chg 2; neg.chg 0 |
| | H-region: length 3; peak value −5.40 |
| | PSG score: −9.80 |
| GvH: | von Heijne's method for signal seq. recognition |
| | GvH score (threshold: −2.1): 2.53 |
| | possible cleavage site: between 35 and 36 |

>>>Seems to have no N-terminal signal peptide

| ALOM: | Klein et al's method for TM region allocation | | | |
|---|---|---|---|---|
| | Init position for calculation: 1 | | | |
| | Tentative number of TMS(s) for the threshold 0.5: 0 | | | |
| | number of TMS(s) . . . fixed | | | |
| | PERIPHERAL Likelihood = 2.97 (at 20) | | | |
| | ALOM score: 2.97 (number of TMSs: 0) | | | |
| MITDISC: | discrimination of mitochondrial targeting seq | | | |
| | R content: | 3 | Hyd Moment (75): | 7.36 |
| | Hyd Moment (95): | 6.26 | G content: | 6 |
| | D/E content: | 1 | S/T content: | 4 |
| | Score: −3.72 | | | |

| | |
|---|---|
| Gavel: | prediction of cleavage sites for mitochondrial preseq |
| | R-2 motif at 49 TRG\|SV |

NUCDISC: discrimination of nuclear localization signals

| | |
|---|---|
| | pat4: none |
| | pat7: none |
| | bipartite: none |
| | content of basic residues: 8.4% |
| | NLS Score: −0.47 |
| KDEL: | ER retention motif in the C-terminus: none |

ER Membrane Retention Signals:

| | |
|---|---|
| | XXRR-like motif in the N-terminus: PGPR |
| | KKXX-like motif in the C-terminus: MNGK |
| SKL: | peroxisomal targeting signal in the C-terminus: none |
| PTS2: | 2nd peroxisomal targeting signal: none |

VAC: possible vacuolar targeting motif: none
RNA-binding motif: none
Actinin-type actin-binding motif:

| | |
|---|---|
| | type 1: none |
| | type 2: none |
| NMYR: | N-myristoylation pattern: none |

Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

--------------------------
Final Results (k = 9/23)

60.9%: mitochondrial
17.4%: cytoplasmic
17.4%: nuclear
4.3%: peroxisomal

>>prediction for CG102348-01 is mit (k = 23)

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU08683 | Human FCTR5a polypeptide | 1 . . . 487 | 485/487 (99%) | 0.0 |
| | sequence - Homo sapiens, 487 | 1 . . . 487 | 487/487 (99%) | |
| | aa. [WO200166747-A2, 13-SEP- | | | |
| | 2001] | | | |
| AAU08684 | Human FCTR5b polypeptide | 1 . . . 487 | 484/487 (99%) | 0.0 |
| | sequence - Homo sapiens, 487 | 1 . . . 487 | 487/487 (99%) | |
| | aa. [WO200166747-A2, 13-SEP- | | | |
| | 2001] | | | |
| AAB23624 | Human secreted protein SEQ ID | 24 . . . 487 | 464/464 (100%) | 0.0 |
| | NO: 48 - Homo sapiens, 464 aa. | 1 . . . 464 | 464/464 (100%) | |
| | [WO200049134-A1, 24-AUG- | | | |
| | 2000] | | | |
| AAB95642 | Human protein sequence SEQ ID | 1 . . . 425 | 423/425 (99%) | 0.0 |
| | NO: 18384 - Homo sapiens, 438 | 1 . . . 425 | 423/425 (99%) | |
| | aa. [EP1074617-A2, 07-FEB- | | | |
| | 2001] | | | |
| ABG65427 | Human albumin fusion protein | 24 . . . 413 | 389/390 (99%) | 0.0 |
| | #2102 - Homo sapiens, 396 aa. | 1 . . . 390 | 390/390 (99%) | |
| | [WO200177137-A1, 18-OCT- | | | |
| | 2001] | | | |

In a BLAST search of public sequence databases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NZP8 | Complement C1r-like | 1 . . . 487 | 487/487 (100%) | 0.0 |
| | proteinase - Homo sapiens | 1 . . . 487 | 487/487 (100%) | |
| | (Human), 487 aa. | | | |
| CAC88678 | Sequence 16 from Patent | 1 . . . 487 | 485/487 (99%) | 0.0 |
| | WO0166747 - Homo sapiens | 1 . . . 487 | 487/487 (99%) | |
| | (Human), 487 aa. | | | |
| Q9H804 | Hypothetical protein - Homo | 1 . . . 425 | 423/425 (99%) | 0.0 |
| | sapiens (Human), 438 aa. | 1 . . . 425 | 423/425 (99%) | |
| AAH35220 | Similar to complement | 189 . . . 483 | 176/303 (58%) | e-108 |
| | component - Homo sapiens | 400 . . . 701 | 226/303 (74%) | |
| | (Human), 705 aa. | | | |
| Q8J012 | Complement component 1, r | 189 . . . 483 | 176/303 (58%) | e-108 |
| | subcomponent - Homo sapiens | 144 . . . 445 | 226/303 (74%) | |
| | (Human), 449 aa (fragment). | | | |

PFam analysis indicates that the NOV3a protein contains the domains shown in the Table 3F.

TABLE 3F
Domain Analysis of NOV3a

| | | | |
|---|---|---|---|
| | | Identities/ | |
| | NOV3a | Similarities for | |
| Pfam Domain | Match Region | the Matched Region | Expect Value |

| | | | |
|---|---|---|---|
| CUB | 40 . . . 160 | 41/131 (31%) | 1.4e-06 |
| | | 88/131 (67%) | |
| trypsin | 245 . . . 479 | 87/270 (32%) | 2.1e-43 |
| | | 174/270 (64%) | |

### Example 4

The NOV4 clone was analyzed, and the nucleotide and encoded polypeptide sequences are shown in Table 4A.

TABLE 4A
NOV4 Sequence Analysis

| | | |
|---|---|---|
| NOV4a, CG125860-02 | SEQ ID NO: 53 | 1880 bp |

| | | |
|---|---|---|
| DNA Sequence | ORF Start: ATG at 24 | ORF Stop: TAA at 1563 |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

NOV4a, CG125860-02

| | | | |
|---|---|---|---|
| Protein Sequence | SEQ ID NO: 54 | 513 aa | MW at 55531.5kD |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | | |
|---|---|---|
| NOV4b, CG125860-01 | SEQ ID NO: 55 | 1787 bp |

| | | |
|---|---|---|
| DNA Sequence | ORF Start: ATG at 54 | ORF Stop: TAA at 1470 |

GCGGAACATTGCCTAGTAGACCCTGAGGCTTTACAACAGTGCCACTGACCCCTATGAGCCTGATGCTG
GATGACCAACCCCCTATGGAGGCCCAGTATGCAGAGGAGGGCCCAGGACCTGGGATCTTCAGAGCAGA
GCCTGGAGACCAGCAGCATCCCATTTCTCAGGCGGTGTGCTGGCGTTCCATGCGACGTGGCTGTGCAG
TGCTGGGAGCCCTGGGGCTGCTGGCCGGTGCAGGTGTTGGCTCATGGCTCCTAGTGCTGTATCTGTGT
CCTGCTGCCTCTCAGCCCATTTCCGGGACCTTGCAGGATGAGGAGATAACTTTGAGCTGCTCAGAGGC
CAGCGCTGAGGAAGCTCTGCTCCCTGCACTTCCCAAAACAGTATCTTTCAGAATAAACAGCGAAGACT
TCTTGCTGGAAGCGCAAGTGAGGGATCAGCCACGCTGGCTCCTGGTCTGCCATGAGGGCTGGAGCCCC
GCCCTGGGGCTGCAGATCTGCTGGAGCCTTGGGCATCTCAGACTCACTCACCACAAGGGAGTAAACCT
CACTGACATCAAACTCAACAGTTCCCAGGAGTTTGCTCAGCTCTCTCCTAGACTGGGAGGCTTCCTGG
AGGAGGCGTGGCAGCCCAGTAGGACTACTGAGGCTGTTAGGAACAACTGCACTTCTGGTCAAGTTGTT
TCCCTCAGATGCTCTGAGTGTGGAGCGAGGCCCCTGGCTTCCCGGATAGTTGGTGGGCAGTCTGTGGC
TCCTGGGCGCTGGCCGTGGCAGGCCAGCGTGGCCCTGGGCTTCCGGCACACGTGTGGGGGCTCTGTGC
TAGCGCCACGCTGGGTGGTGACTGCTGCACATTGTATGCACAGTTTCAGGCTGGCCCGCCTGTCCAGC
TGGCGGGTTCATGCGGGGCTGGTCAGCCACAGTGCCGTCAGGCCCCACCAAGGGGCTCTGGTGGAGAG
GATTATCCCACACCCCCTCTACAGTGCCCAGAATCATGACTACGACGTCGCCCTCCTGAGGCTCCAGA
CCGCTCTCAACTTCTCAGACACTGTGGGCGCTGTGTGCCTGCCGGCCAAGGAACAGCATTTTCCGAAG
GGCTCGCGGTGCTGGGTGTCTGGCTGGGGCCACACCCACCCTAGCCATACTTACAGCTCGGATATGCT
CCAGGACACGGTGGTGCCCCTGCTCAGCACTCAGCTCTGCAACAGCTCTTGCGTGTACAGCGGAGCCC
TCACCCCCCGCATGCTTTGCGCTGGCTACCTGGACGGAAGGGCTGATGCATGCCAGGGAGATAGCGGG
GGCCCCCTAGTGTGCCCAGATGGGGACACATGGCGCCTAGTGGGGGTGGTCAGCTGGGGGCGTGGCTG
CGCAGAGCCCAATCACCCAGGTGTCTACGCCAAGGTAGCTGAGTTTCTGGACTGGATCCATGACACTG
CTCAGGTGAGTGTGGGGGCAGGAGTAGGGCAGGGAGATTTCTAAAGGACCTGCCCTCGAATGCAAGGA
ACCTTACCCCTTAGGCCCGGGCCCTGCTGGGGACTGGGGAGGGTGCTAGGACATATTCCCCAGAGTGA
GTGGAGGAAGAAGTGAAGCTTAAACATGGAATCCATTGGATTTCTATCAGTTTAAGGATGAACTGGGT
AAGAGTATGCCTGAGTTTGTATCCCAGATCTACCATTTCCTGTGTCGACCTTTGGCAAATTTCTAACT
TTGTTAAACCTTAATTTCCTGATAATAACCATGATGGCTACTTATATGCTATTGTTATATGCTATTAA
ATAAGACCCGTACAATGCC

NOV4b, CG125860-01

| | | | |
|---|---|---|---|
| Protein Sequence | SEQ ID NO: 56 | 472 aa | MW at 50916.3kD |

MSLMLDDQPPMEAQYAEEGPGPGIFPAEPGDQQHPISQAVCWRSMRRGCAVLGALGLLAGAGVGSWLL
VLYLCPAASQPISGTLQDEEITLSCSEASAEEALLPALPKTVSFRINSEDFLLEAQVRDQPRWLLVCH
EGWSPALGLQICWSLGHLRLTHHKGVNLTDIKLNSSQEFAQLSPRLGGFLEEAWQPSRTTEAVRNNCT
SGQVVSLRCSECGARPLASRIVGGQSVAPGRWPWQASVALGFRHTCGGSVLAPRWVVTAAHCMHSFRL
ARLSSWRVHAGLVSHSAVRPHQGAINERIIPHPLYSAQNHDYDVALLRLQTALNFSDTVGAVCLPAKE
QHFPKGSRCWVSGWGHTHPSHTYSSDMLQDTVVPLLSTQLCNSSCVYSGALTPRNLCAGYLDGRADAC
QGDSGGPLVCPDGDTWRLVGVVSWGRGCAEPNHPGVYAKVAEFLDWIHDTAQVSVGAGVGQGDF

| | |
|---|---|
| SEQ ID NO: 57 | 1880 bp |

| | | |
|---|---|---|
| NOV4c, SNP13376012 of | ORF Start: ATG at 24 | ORF Stop: TAA at 1563 |

| | | |
|---|---|---|
| CG125860-02, DNA Sequence | SNP Pos: 36 | SNP Change: C to T |

TTACAACAGTGCCACTGACCCCTATGAGCCTGATGTTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGcTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4c, SNP13376012 of | SEQ ID NO: 58 | MW at 55531.5kD |

| | | | |
|---|---|---|---|
| CG125860-02, Protein Sequence | SNP Pos: 5 | 513 aa | SNP Change: Leu to Leu |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPAIPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 59 | 1880 bp |

| | | |
|---|---|---|
| NOV4d, SNP13376014 of | ORF Start: ATG at 24 | ORF Stop: TAA at 1563 |

| | | |
|---|---|---|
| CG125860-02, DNA Sequence | SNP Pos: 265 | SNP Change: G to A |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCAACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCcTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4d, SNP13376014 of | SEQ ID NO: 60 | MW at 55503.5kD |

| | | | |
|---|---|---|---|
| CG125860-02, Protein Sequence | SNP Pos: 81 | 513 aa | SNP Change: Arg to Gln |

MSLMLDDQPPMEAQYAEEGPGPGIFRAEPGDQQHPSRPDWAIGEMTGWGQWRAIILHSPDPPWGQPHM
IDVSQAVCWRSMQRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 61 | 1880 bp |

| | | |
|---|---|---|
| NOV4e, SNP13376015 of | ORF Start: ATG at 24 | ORF Stop: TAA at 1563 |

| | | |
|---|---|---|
| CG125860-02, DNA Sequence | SNP Pos: 827 | SNP Change: T to C |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGcTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGCGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4e, SNP13376015 of | SEQ ID NO: 62 | MW at 55531.5kD |

| | | | |
|---|---|---|---|
| CG125860-02, Protein Sequence | SNP Pos: 268 | 513 aa | SNP Change: Gly to Gly |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCNHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 63 | 1880 bp |

| | | |
|---|---|---|
| NOV4f, SNP13376016 of | ORF Start: ATG at 24 | ORF Stop: TAA at 1563 |

| | | |
|---|---|---|
| CG125860-02, DNA Sequence | SNP Pos: 830 | SNP Change: G to T |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGTCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4f, SNP13376016 of | SEQ ID NO: 64 | MW at 55531.5kD |

| | | | |
|---|---|---|---|
| CG125860-02, Protein Sequence | SNP Pos: 269 | 513 aa | SNP Change: Gly to Gly |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRHLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 65 | 1880 bp |

| | | |
|---|---|---|
| NOV4g, SNP13376017 of | ORF Start: ATG at 24 | ORF Stop: TAA at 1563 |

| | | |
|---|---|---|
| CG125860-02, DNA Sequence | SNP Pos: 836 | SNP Change: T to C |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCITGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCCGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4g, SNP13376017 of | SEQ ID NO: 66 | MW at 55531.5kD |

| | | | |
|---|---|---|---|
| CG125860-02, Protein Sequence | SNP Pos: 271 | 513 aa | SNP Change: Ser to Ser |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 67 | 1880 bp |

| | | |
|---|---|---|
| NOV4h, SNP13376011 of | ORF Start: ATG at 24 | ORF Stop: TAA at 1563 |

| | | |
|---|---|---|
| CG125860-02, DNA Sequence | SNP Pos: 869 | SNP Change: C to T |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGTGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4h, SNP13376011 of | SEQ ID NO: 68 | MW at 55531.5kD |

| | | | |
|---|---|---|---|
| CG125860-02, Protein Sequence | SNP Pos: 282 | 513 aa | SNP Change: Ser to Ser |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTOLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 69 | 1880 bp |

| | | |
|---|---|---|
| NOV4i, SNP13376018 of | ORF Start: ATG at 24 | ORF Stop: TAA at 1563 |

| | | |
|---|---|---|
| CG125860-02, DNA Sequence | SNP Pos: 975 | SNP Change: A to G |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCGGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4i, SNP13376018 of | SEQ ID NO: 70 | MW at 55501.5kD |

| | | | |
|---|---|---|---|
| CG125860-02, Protein Sequence | SNP Pos: 318 | 513 aa | SNP Change: Ser to Gly |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSGWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

| | |
|---|---|
| SEQ ID NO: 71 | 1880 bp |

| | | |
|---|---|---|
| NOV4j, SNP13382467 of | ORF Start: ATG at 24 | ORF Stop: TAA at 1563 |

| | | |
|---|---|---|
| CG125860-02, DNA Sequence | SNP Pos: 1269 | SNP Change: C to T |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GTCTGCCATGAGGGCTGGAGCCCCGCCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCAGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCTTGCTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4j, SNP13382467 of | SEQ ID NO: 72 | MW at 55531.5kD |

| | | | |
|---|---|---|---|
| CG125860-02, Protein Sequence | SNP Pos: 416 | 513 aa | SNP Change: Leu to Leu |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLLSTQLCNSSCVYSGALTPRMLCAGYLDGRADACQGDSGGPLVCPDGDTWRLVGVVSWGRG
CAEPMHPGVYAKVAEFLDWIHDTAQVSVGAGVGQGDF

| | |
|---|---|
| SEQ ID NO: 73 | 1880 bp |

| | | |
|---|---|---|
| NOV4k, SNP13382466 of | ORF Start: ATG at 24 | ORF Stop: TAA at 1563 |

| | | |
|---|---|---|
| CG125860-02, DNA Sequence | SNP Pos: 1272 | SNP Change: C to T |

TTACAACAGTGCCACTGACCCCTATGAGCCTGATGCTGGATGACCAACCCCCTATGGAGGCCCAGTAT
GCAGAGGAGGGCCCAGGACCTGGGATCTTCAGAGCAGAGCCTGGAGACCAGCAGCATCCCAGTAGGCC
AGACTGGGCCATAGGGGAAATGACAGGGTGGGGACAGTGGAGGGCAATCATCCTACATTCCCCGGATC
CTCCTTGGGGTCAGCCCCACATGATTGATGTTTCTCAGGCAGTGTGCTGGCGTTCCATGCGACGTGGC
TGTGCAGTGCTGGGAGCCCTGGGGCTGCTGGCCGGTGCAGGTGTTGGCTCATGGCTCCTAGTGCTGTA
TCTGTGTCCTGCTGCCTCTCAGCCCATTTCCGGGACCTTGCAGGATGAGGAGATAACTTTGAGCTGCT
CAGAGGCCAGCGCTGAGGAAGCTCTGCTCCCTGCACTTCCCAAAACACCTGCACTTCCCAAAACAGTA
TCTTTCAGAATAAACAGCGAAGACTTCTTGCTGGAAGCGCAAGTGAGGGATCAGCCACGCTGGCTCCT
GGTCTGCCATGAGGGCTGGAGCCCCGCCCTGGGGCTGCAGATCTGCTGGAGCCTTGGGCATCTCAGAC
TCACTCACCACAAGGGAGTAAACCTCACTGACATCAAACTCAACAGTTCCCAGGAGTTTGCTCTGCTC
TCTCCTAGACTGGGAGGCTTCCTGGAGGAGGCGTGGCAGCCCAGTAGGACTACTGAGGCTGTTAGGAA
CAACTGCACTTCTGGTCAAGTTGTTTCCCTCAGATGCTCTGAGTGTGGAGCGAGGCCCCTGGCTTCCC
GGATAGTTGGTGGGCAGTCTGTGGCTCCTGGGCGCTGGCCGTGGCAGGCCAGCGTGGCCCTGGGCTTC
CGGCACACGTGTGGGGGCTCTGTGCTAGCGCCACGCTGGGTGGTGACTGCTGCACATTGTATGCACAG
TTTCAGGCTGGCCCGCCTGTCCAGCTGGCGGGTTCATGCGGGGCTGGTCAGCCACAGTGCCGTCAGGC
CCCACCAAGGGGCTCTGGTGGAGAGGATTATCCCACACCCCCTCTACAGTGCCCAGAATCATGACTAC
GACGTCGCCCTCCTGAGGCTCCAGACCGCTCTCAACTTCTCAGACACTGTGGGCGCTGTGTGCCTGCC
GGCCAAGGAACAGCATTTTCCGAAGGGCTCGCGGTGCTGGGTGTCTGGCTGGGGCCACACCCACCCTA
GCCATACTTACAGCTCGGATATGCTCCAGGACACGGTGGTGCCCCTGTTCAGCACTCAGCTCTGCAAC
AGCTCTTGCGTGTACAGCGGAGCCCTCACCCCCCGCATGCTTTGCGCTGGCTACCTGGACGGAAGGGC
TGATGCATGCCAGGGAGATAGCGGGGGCCCCCTAGTGTGCCCAGATGGGGACACATGGCGCCTAGTGG
GGGTGGTCAGCTGGGGGCGTGGCTGCGCAGAGCCCAATCACCCAGGTGTCTACGCCAAGGTAGCTGAG
TTTCTGGACTGGATCCATGACACTGCTCAGGTGAGTGTGGGGGCAGGAGTAGGGCAGGGAGATTTCTA
AAGGACCTGCCCTCGAATGCAAGGAACCTTACCCCTTAGGCCCGGGCCCTGCTGGGGACTGGGGAGGG
TGCTAGGACATATTCCCCAGAGTGAGTGGAGGAAGAAGTGAAGCTTAAACATGGAATCCATTGGATTT
CTATCAGTTTAAGGATGAACTGGGTAAGAGTATGCCTGAGTTTGTATCCCAGATCTACCATTTCCTGT
GTCGACCTTTGGCAAATTTCTAACTTTGTTAAACCTTAATTTCCTGATAATAACCATGATGGCTACTT
ATATGCTATTGTTATATGCTATTAAATAAGACCCGTACAATGCC

| | | |
|---|---|---|
| NOV4k, SNP13382466 of | SEQ ID NO: 74 | MW at 55565.5kD |

| | | | |
|---|---|---|---|
| CG125860-02, Protein Sequence | SNP Pos: 417 | 513 aa | SNP Change: Leu to Phe |

MSLMLDDQPPMEAQYAEEGPGPGIFRAEPGDQQHPSRPDWAIGEMTGWGQWRAIILHSPDPPWGQPHM
IDVSQAVCWRSMRRGCAVLGALGLLAGAGVGSWLLVLYLCPAASQPISGTLQDEEITLSCSEASAEEA
LLPALPKTPALPKTVSFRINSEDFLLEAQVRDQPRWLLVCHEGWSPALGLQICWSLGHLRLTHHKGVN
LTDIKLNSSQEFAQLSPRLGGFLEEAWQPSRTTEAVRNNCTSGQVVSLRCSECGARPLASRIVGGQSV
APGRWPWQASVALGFRHTCGGSVLAPRWVVTAAHCMHSFRLARLSSWRVHAGLVSHSAVRPHQGALVE
RIIPHPLYSAQNHDYDVALLRLQTALNFSDTVGAVCLPAKEQHFPKGSRCWVSGWGHTHPSHTYSSDM
LQDTVVPLFSTQLCNSSCVYSGALTPRMLCAGYLDGRADACQGDSGGPLVCPDGDTWRLVGVVSWGRG
CAEPNHPGVYAKVAEFLDWIHDTAQVSVGAGVGQGDF

A ClustalW comparison of the above protein sequences yields the following sequence alignment shown in Table 4B.

TABLE 4B
Comparison of the NOV4 protein sequences.

| | |
|---|---|
| NOV4a | MSLMLDDQPPMEAQYAEEGPGPGIFRAEPGDQQHPSRPDWAIGEMTGWGQWRAIILHSPD |
| NOV4b | MSLMLDDQPPMEAQYAEEGPGPGIFRAEPGDQQHP------------------------- |
| NOV4a | PPWGQPHMIDVSQAVCWRSMRRGCAVLGALGLLAGAGVGSWLLVLYLCPAASQPISGTLQ |
| NOV4b | ----------ISQAVCWRSMRRGCAVLGALGLLAGAGVGSWLLVLYLCPAASQPISGTLQ |
| NOV4a | DEEITLSCSEASAEEALLPALPKTPALPKTVSFRINSEDFLLEAQVRDQPRWLLVCHEGW |
| NOV4b | DEEITLSCSEASAEEALLPALPKT------VSFRINSEDFLLEAQVRDQPRWLLVCHEGW |
| NOV4a | SPALGLQICWSLGHLRLTHHKGVNLTDIKLNSSQEFAQLSPRLGGFLEEAWQPSRTTEAV |
| NOV4b | SPALGLQICWSLGHLRLTHHKGVNLTDIKLNSSQEFAQLSPRLGGFLEEAWQPSRTTEAV |
| NOV4a | RNNCTSGQVVSLRCSECGARPLASRIVGGQSVAPGRWPWQASVALGFRHTCGGSVLAPRW |
| NOV4b | RNNCTSGQVVSLRCSECGARPLASRIVGGQSVAPGRWPWQASVALGFRHTCGGSVLAPRW |
| NOV4a | VVTAAHCMHSFRLARLSSWRVHAGLVSHSAVRPHQGALVERIIPHPLYSAQNHDYDVALL |
| NOV4b | VVTAAHCMHSFRLARLSSWRVHAGLVSHSAVRPHQGALVERIIPHPLYSAQNHDYDVALL |
| NOV4a | RLQTALNFSDTVGAVCLPAKEQHFPKGSRCWVSGWGHTHPSHTYSSDMLQDTVVPLLSTQ |
| NOV4b | RLQTALNFSDTVGAVCLPAKEQHFPKGSRCWVSGWGHTHPSHTYSSDMLQDTVVPLLSTQ |
| NOV4a | LCNSSCVYSGALTPRMLCAGYLDGRADACQGDSGGPLVCPDGDTWRLVGVVSWGRGCAEP |
| NOV4b | LCNSSCVYSGALTPRMLCAGYLDGRADACQGDSGGPLVCPDGDTWRLVGVVSWGRGCAEP |
| NOV4a | NHPGVYAKVAEFLDWIHDTAQVSVGAGVGQGDF |
| NOV4b | NHPGVYAKVAEFLDWIHDTAQVSVGAGVGQGDF |
| NOV4a | (SEQ ID NO: 54) |
| NOV4b | (SEQ ID NO: 56) |

Further analysis of the NOV4a protein yielded the following properties shown in Table 4C.

TABLE 4C
Protein Sequence Properties NOV4a

| | |
|---|---|
| SignalP analysis: | No Known Signal Sequence Indicated |
| PSORT II analysis: | |

| | |
|---|---|
| PSG: | a new signal peptide prediction method |
| | N-region: length 7; pos.chg 0; neg.chg 2 |
| | H-region: length 4; peak value 0.00 |
| | PSG score: −4.40 |
| GvH: | von Heijne's method for signal seq. recognition |
| | GvH score (threshold: −2.1): −12.80 |
| | possible cleavage site: between 13 and 14 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 1 |
| | Number of TMS(s) for threshold 0.5: 1 |
| | INTEGRAL Likelihood = −3.40 Transmembrane 89-105 |
| | PERIPHERAL Likelihood = 5.57 (at 173) |
| | ALOM score: −3.40 (number of TMSs: 1) |
| MTOP: | Prediction of membrane topology (Hartmann et al.) |
| | Center position for calculation: 96 |
| | Charge difference: −8.0 C(−6.0) − N( 2.0) |
| | N >= C: N-terminal side will be inside |

>>> membrane topology: type 2 (cytoplasmic tail 1 to 89)

| MITDISC: | discrimination of mitochondrial targeting seq | | | |
|---|---|---|---|---|
| | R content: | 0 | Hyd Moment (75): | 8.83 |
| | Hyd Moment(95): | 8.90 | G content: | 0 |
| | DIE content: | 2 | S/T content: | 1 |
| | Score: −6.04 | | | |

| | |
|---|---|
| Gavel: | prediction of cleavage sites for mitochondrial preseq |
| | cleavage site motif not found |

NUCDISC: discrimination of nuclear localization signals

| | pat4: none |
|---|---|
| | pat7: none |
| | bipartite: none |
| | content of basic residues: 7.2% |
| | NLS Score: −0.47 |
| KDEL: | ER retention motif in the C-terminus: none |

ER Membrane Retention Signals: none

| | |
|---|---|
| SKL: | peroxisomal targeting signal in the C-terminus: none |
| PTS2: | 2nd peroxisomal targeting signal: none |
| VAC: | possible vacuolar targeting motif: none |

RNA-binding motif: none
Actinin-type actin-binding motif:

| | type 1: none |
|---|---|
| | type 2: none |
| NMYR: | N-myristoylation pattern : none |

Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

--------------------------
Final Results (k = 9/23):

```
34.8%: mitochondrial
26.1%: cytoplasmic
17.4%: Golgi
  8.7%: endoplasmic reticulum
  4.3%: extracellular, including cell wall
  4.3%: nuclear
  4.3%: vesicles of secretory system
```

>> prediction for CG125860-02 is mit (k = 23)

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG76906 | Human hepsin/plasma | 1 . . . 513 | 470/513 (91%) | 0.0 |
| | transmembrane serine protease- | 1 . . . 472 | 472/513 (91%) | |
| | like protein - Homo sapiens, 472 | | | |
| | aa. [WO200233087-A2, 25-APR- | | | |
| | 2002] | | | |
| AAU82752 | Amino acid sequence of novel | 1 . . . 504 | 451/504 (89%) | 0.0 |
| | human protease #51 - Homo | 1 . . . 456 | 453/504 (89%) | |
| | sapiens, 457 aa. [WO200200860- | | | |
| | A2, 03-JAN-2002] | | | |
| AAB11699 | Human serine protease BSSP2 | 1 . . . 504 | 451/504 (89%) | 0.0 |
| | (hBSSP2), SEQ ID NO:10 - Homo | 1 . . . 456 | 453/504 (89%) | |
| | sapiens, 457 aa. [WO200031272- | | | |
| | A1, 02-JUN-2000] | | | |
| AAB08950 | Human secreted protein sequence | 69 . . . 504 | 417/436 (95%) | 0.0 |
| | encoded by gene 22 SEQ ID | 57 . . . 479 | 419/436 (95%) | |
| | NO:107 - Homo sapiens, 480 aa. | | | |
| | [WO200017222-A1, 30-MAR- | | | |
| | 2000] | | | |
| AAB08912 | Human secreted protein sequence | 80 . . . 504 | 409/425 (96%) | 0.0 |
| | encoded by gene 22 SEQ ID | 1 . . . 412 | 410/425 (96%) | |
| | NO:69 - Homo sapiens, 414 aa. | | | |
| | [WO200017222-A1, 30-MAR- | | | |
| | 2000] | | | |

In a BLAST search of public sequence databases, the NOV4a protein was found to have homology to the proteins shown in the BLASTP data in Table 4E.

TABLE 4E
Public BLASTP Results for NOV4a

| | | | Identities/ | |
|---|---|---|---|---|
| | NOV4a | Similarities | | |
| Protein | | Residues/ | for the | |
| Accession | Protein/ | Match | Matched | Expect |
| Number | Organism/Length | Residues | Portion | Value |
| Q9H3S3 | Transmembrane pro- | 1 . . . 504 | 451/504 | 0.0 |
| | tease, serine 5 (EC | 1 . . . 456 | (89%) | |
| | 3.4.21.-) (Spinesin) - | | 453/504 | |
| | Homo sapiens | | (89%) | |
| | (Human), 457 aa. | | | |
| Q8CJ17 | Adrenal mitochondrial | 11 . . . 502 | 360/492 | 0.0 |
| | protease long variant - | 1 . . . 444 | (73%) | |
| | Rattus norvegicus | | 393/492 | |
| | (Rat), 445 aa. | | (79%) | |
| Q8CDR0 | Transmembrane pro- | 1 . . . 502 | 359/502 | 0.0 |
| | tease - Mus musculus | 1 . . . 454 | (71%) | |
| | (Mouse), 455 aa. | | 394/502 | |
| | | | (77%) | |
| Q9ER04 | Transmembrane pro- | 1 . . . 502 | 359/502 | 0.0 |
| | tease, serine 5 (EC | 1 . . . 454 | (71%) | |
| | 3.4.21.-) (Spinesin) - | | 394/502 | |
| | Mus musculus | | (77%) | |
| | (Mouse), 455 aa. | | | |
| Q8CJ16 | Adrenal mitochondrial | 151 . . . 502 | 289/352 | e-177 |
| | protease short variant - | 26 . . . 370 | (82%) | |
| | Rattus norvegicus | | 312/352 | |
| | (Rat), 371 aa. | (88%) | | |

PFam analysis indicates that the NOV4a protein contains the domains shown in the Table 4F.

TABLE 4F
Domain Analysis of NOV4a

| | | Identities/ | |
|---|---|---|---|
| | NOV4a | Similarities for | |
| Pfam Domain | Match Region | the Matched Region | Expect Value |
| trypsin | 266. . . 496 | 111/265 (42%) | 3.9e-87 |
| | | 198/265 (75%) | |

### Example 5

The NOV5 clone was analyzed, and the nucleotide and encoded polypeptide sequences are shown in Table 5A.

TABLE 5A
NOV5 Sequence Analysis

| | | |
|---|---|---|
| NOV5a, CG155759-02 | SEQ ID NO: 75 | 941 bp |

| | | |
|---|---|---|
| DNA Sequence | ORF Start: ATG at 4 | ORF Stop: TAG at 931 |

GACATGGAACAGGATAATACAACATTGCTGACAGAGTTTGTTCTCACAGGACTTACATATCAGCCAGA
GTGGAAAATGCCCCTGTTCTTGGTGTTCTTGGTGATCTATCTCATCACTATTGTGTGGAACCTTGGTC
TGATTGCTCTTATCTGGAATGACCCACAACTTCACATCCCCATGTACTTTTTTCTTGGGAGTTTAGCC
TTTGTTGATGCTTGGATATCTTCCACAGTAACTCCCAAAATGTTGGTTAATTTCTTGGCCAAAAACAG
GATGATATCTCTGTCTGAATGCATGATTCAATTTTTTTCCTTTGCATTTGGTGGAACTACAGAATGTT
TTCTCTTGGCAACAATGGCATATGATCGCTATGTAGCCATATGCAAACCTTTACTATATCCAGTGATT
ATGAACAATTCACTATGCATACGGCTGTTAGCCTTCTCATTTTTAGGTGGCTTCCTCCATGCCTTAAT
TCATGAAGTCCTTATATTCAGATTAACCTTCTGCAATTCTAACATAATACATCATTTTTACTGTGATA
TTATACCACTGTTTATGATTTCCTGTACTGACCCTTCTATTAATTTTCTAATGGTTTTTATTTTGTCT
GGCTCAATTCAGGTATTCACCATTGTGACAGTTCTTAATTCTTACACATTTGCTCTTTTCACAATCCT
AAAAAAGAAGTCTGTTAGAGGCGTAAGGAAAGCCTTTTCCACCTGTGGAGCCCATCTCTTATCTGTCT
CTTTATATTATGGCCCACTTATCTTCATGTATTTGCGCCCTGCATCTCCACAAGCAGATGACCAGGAT
ATGATAGACTCTGTCTTTTATACAATCATAATTCCTTTGCTAAATCCCATTATCTACAGTCTGAGAAA
TAAACAAGTAATAGATTCATTCACAAAAATGGTAAAAAGAAATGTTTAGATTTCATA

NOV5a, CG155759-02

| | | | |
|---|---|---|---|
| Protein Sequence | SEQ ID NO: 76 | 309 aa | MW at 35396.8kD |

MEQDNTTLLTEFVLTGLTYQPEWKMPLFLVFLVIYLITIVWNLGLIALIWNDPQLHIPMYFFLGSLAF
VDAWISSTVTPKMLVNFLAKNRMISLSECMIQFFSFAFGGTTECFLLATMAYDRYVAICKPLLYPVIM
NNSLCIRLLAFSFLGGFLHALIHEVLIFRLTFCNSNIIHHFYCDIIPLFMISCTDPSINFLMVFILSG
SIQVFTIVTVLNSYTFALFTILKKKSVRGVRKAFSTCGAHLLSVSLYYGPLIFMYLRPASPQADDQDM
IDSVFYTIIIPLLNPIIYSLRNKQVIDSFTKMVKRNV

| | | |
|---|---|---|
| NOV5b, CG155759-01 | SEQ ID NO: 77 | 941 bp |

| | | |
|---|---|---|
| DNA Sequence | ORF Start: ATG at 4 | ORF Stop: TAG at 931 |

GACATGGAACAGGATAATACAACATTGCTGACAGAGTTTGTTCTCACAGGACTTACATATCAGCCAGA
GTGGAAAATGCCCCTGTTCTTGGTGTTCTTGGTGATCTATCTCATCACTATTGTGTGGAACCTTGGTC
TGATTGCTCTTATCTGGAATGACCCACAACTTCACATCCCCATGTACTTTTTTCTTGGGAGTTTAGCC
TTTGTTGATGCTTGGATATCTTCCACAGTAACTCCCAAAATGTTGGTTAATTTCTTGGCCAAAAACAG
GATGATATCTCTGTCTGAATGCATGATTCAATTTTTTTCCTTTGCATTTGGTGGAACTACAGAATGTT
TTCTCTTGGCAACAATGGCATATGATCGCTATGTAGCCATATGCAAACCTTTACTATATCCAGTGATT
ATGAACAATTCACTATGCATACGGCTGTTAGCCTTCTCATTTTTAGGTGGCTTCCTCCATGCCTTAAT
TCATGAAGTCCTTATATTCAGATTAACCTTCTGCAATTCTAACATAATACATCATTTTTACTGTGATA
TTATACCACTGTTTATGATTTCCTGTACTGACCCTTCTATTAATTTTCTAATGGTTTTTATTTTGTCT
GGCTCAATTCAGGTATTCACCATTGTGACAGTTCTTAATTCTTACACATTTGCTCTTTTCACAATCCT
AAAAAAGAAGTCTGTTAGAGGCGTAAGGAAAGCCTTTTCCACCTGTGGAGCCCATCTCTTATCTGTCT
CTTTATATTATGGCCCACTTATCTTCATGTATTTGCGCCCTGCATCTCCACAAGCAGATGACCAAGAT
ATGATAGACTCTGTCTTTTATACAATCATAATTCCTTTGCTAAATCCCATTATCTACAGTCTGAGAAA
TAAACAAGTAATAGATTCATTCACAAAAATGGTAAAAAGAAATGTTTAGATTTCATA

NOV5b, CG155759-01

| | | | |
|---|---|---|---|
| Protein Sequence | SEQ ID NO: 78 | 309 aa | MW at 35396.8kD |

MEQDNTTLLTEFVLTGLTYQPEWKMPLFLVFLVIYLITIVWNLGLIALIWNDPQLHIPMYFFLGSLAF
VDAWISSTVTPKMLVNFLAKNRNISLSECMIQFFSFAFGGTTECFLLATMAYDRYVAICKPLLYPVIM
NNSLCIRLLAFSFLGGFLHALIHEVLIFRLTFCNSNIIHHFYCDIIPLFMISCTDPSINFLMVFILSG
SIQVFTIVTVLNSYTFALFTILKKKSVRGVRKAFSTCGAHLLSVSLYYGPLIFMYLRPASPQADDQDM
IDSVFYTIIIPLLNPIIYSLRNKQVIDSFTKMVKRNV

A ClustalW comparison of the above protein sequences yields the following sequence alignment shown in Table 5B.

TABLE 5B
Comparison of the NOV5 protein sequences.

| | |
|---|---|
| NOV5a | MEQDNTTLLTEFVLTGLTYQPEWKMPLFLVFLVIYLITIVWNLGLIALIWNDPQLHIPMY |
| NOV5b | MEQDNTTLLTEFVLTGLTYQPEWKMPLFLVFLVIYLITIVWNLGLIALIWNDPQLHIPMY |
| NOV5a | FFLGSLAFVDAWISSTVTPKMLVNFLAKNRMISLSECMIQFFSFAFGGTTECFLLATMAY |
| NOV5b | FFLGSLAFVDAWISSTVTPKMLVNFLAKNRMISLSECMIQFFSFAFGGTTECFLLATMAY |
| NOV5a | DRYVAICKPLLYPVIMNNSLCIRLLAFSFLGGFLHALIHEVLIFRLTFCNSNIIHHFYCD |
| NOV5b | DRYVAICKPLLYPVIMNNSLCIRLLAFSFLGGFLHALIHEVLIFRLTFCNSNIIHHFYCD |
| NOV5a | IIPLFMISCTDPSINFLMVFILSGSIQVFTIVTVLNSYTFALFTILKKKSVRGVRKAFST |
| NOV5b | IIPLFMISCTDPSINFLMVFILSGSIQVFTIVTVLNSYTFALFTILKKKSVRGVRKAFST |
| NOV5a | CGAHLLSVSLYYGPLIFMYLRPASPQADDQDMIDSVFYTIIIPLLNPIIYSLRNKQVIDS |
| NOV5b | CGAHLLSVSLYYGPLIFMYLRPASPQADDQDMIDSVFYTIIIPLLNPIIYSLRNKQVIDS |
| NOV5a | FTKMVKRNV |
| NOV5b | FTKMVKRNV |
| NOV5a | (SEQ ID NO: 76) |
| NOV5b | (SEQ ID NO: 78) |

Further analysis of the NOV5a protein yielded the following properties shown in Table 5C.

TABLE 5C
Protein Sequence Properties NOV5a

```
Signa1P analysis: Cleavage site between residues 52 and 53
PSORT II analysis:
PSG: a new signal peptide prediction method
    N-region: length 11; pos. chg 0; neg. chg 3
    H-region: length 10; peak value 0.00
    PSG score: −4.40
GvH: von Heijne's method for signal seq. recognition
    GvH score (threshold: −2.1): −3.77
    possible cleavage site: between 38 and 39
>>> Seems to have no N-terminal signal peptide
ALOM: Klein et al's method for TM region allocation
    Init position for calculation: 1
    Tentative number of TMS(s) for the threshold 0.5: 5
    INTEGRAL Likelihood = −9.24 Transmembrane 25-41
    INTEGRAL Likelihood = 0.37 Transmernbrane 57-73
    INTEGRAL Likelihood = 31 2.02 Transmembrane 142-158
    INTEGRAL Likelihood = 31 5.79 Transmembrane 196-212
    INTEGRAL Likelihood = 31 1.70 Transmembrane 276-292
    PERIPHERAL Likelihood = 0.90 (at 173)
    ALOM score: −9.24 (number of TMSs: 5)
MTOP: Prediction of membrane topology (Hartmann et al.)
    Center position for calculation: 32
    Charge difference: 0.5 C(−0.5) − N(−1.0).
    C > N: C-terminal side will be inside
>>> membrane topology: type 3b
MITDISC: discrimination of mitochondrial targeting seq
    R content: 0 Hyd Moment (75): 6.20
    Hyd Moment(95): 3.11 G content: 0
    D/E content: 2 SIT content: 0
    Score: −7.39
Gavel: prediction of cleavage sites for mitochondrial preseq
    cleavage site motif not found
NUCDISC: discrimination of nuclear localization signals
    pat4: none
    pat7: none
    bipartite: none
    content of basic residues: 6.5%
    NLS Score: −0.47
KDEL: ER retention motif in the C-terminus: none
ER Membrane Retention Signals:
    KKXX-like motif in the C-terminus: VKRN
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 94.1
COIL: Lupas's algorithm to detect coiled-coil regions
    total: 0 residues
--------------------------
Final Results (k = 9/23)
    33.3%: endoplasmic reticulum
    11.1%: mitochondrial
    11.1%: Golgi
    11.1%: vacuolar
    11.1%: nuclear
    11.1%: vesicles of secretory system
    11.1%: cytoplasmic
>> prediction for CG155759-02 is end (k=9)
```

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| ABP72221 | Human G-protein coupled | 1 . . . 309 | 309/309 (100%) | e-179 |
| | receptor GCREC-15 (olfactory | 1 . . . 309 | 309/309 (100%) | |
| | receptor) - Homo sapiens, 309 aa. | | | |
| | [WO2003000859-A2, 03-JAN- | | | |
| | 2003] | | | |
| AAU85253 | G-coupled olfactory receptor | 1 . . . 309 | 309/309 (100%) | e-179 |
| | #114 - Homo sapiens, 314 aa. | 6 . . . 314 | 309/309 (100%) | |
| | [WO200198526-A2, 27-DEC- | | | |
| | 2001] | | | |
| AAU95610 | Human olfactory and pheromone | 1 . . . 309 | 309/309 (100%) | e-179 |
| | G protein-coupled receptor #97 - | 6 . . . 314 | 309/309 (100%) | |
| | Homo sapiens, 314 aa. | | | |
| | [WO200224726-A2, 28-MAR- | | | |
| | 2002] | | | |
| AAB71190 | Human GPCRX protein SEQ ID | 1 . . . 309 | 309/309 (100%) | e-179 |
| | 56 - Homo sapiens, 309 aa. | 1 . . . 309 | 309/309 (100%) | |
| | [WO200250275-A2, 27-JUN- | | | |
| | 2002] | | | |
| ABP95748 | Human GPCR polypeptide SEQ | 1 . . . 309 | 309/309 (100%) | e-179 |
| | ID NO 306- Homo sapiens, 314 | 6 . . . 314 | 309/309 (100%) | |
| | aa. [WO200216548-A2, 28-FEB- | | | |
| | 2002] | | | |

In a BLAST search of public sequence databases, the NOV5a protein was found to have homology to the proteins shown in the BLASTP data in Table 5E.

TABLE 5E
Public BLASTP Results for NOV5a

| | | | Identities/ | |
|---|---|---|---|---|
| | | NOV5a | Similarities | |
| Protein | | Residues/ | for the | |
| Accession | Protein/ | Match | Matched | Expect |
| Number | Organism/Length | Residues | Portion | Value |
| Q8NGV7 | Seven transmembrane | 1 . . . 309 | 309/309 | e-179 |
| | helix receptor - | 6 . . . 314 | (100%) | |
| | Homo sapiens | | 309/309 | |
| | (Human), 314 aa. | | (100%) | |
| Q8VG48 | Olfactory receptor | 4 . . . 308 | 232/305 | e-141 |
| | MOR183-1 - | 4 . . . 308 | (76%) | |
| | Mus musculus | | 272/305 | |
| | (Mouse), 309 aa. | | (89%) | |
| Q8VEX5 | Olfactory receptor | 1 . . . 309 | 239/309 | e-139 |
| | MOR183-1 - | 1 . . . 309 | (77%) | |
| | Mus musculus | | 270/309 | |
| | (Mouse), 309 aa. | | (87%) | |
| CAD- | Sequence 195 from | 1 . . . 309 | 241/309 | e-139 |
| 37583 | Patent WO0224726 - | 17 . . . 325 | (77%) | e-139 |
| | Homo sapiens | | 267/309 | |
| | (Human), 325 aa. | | (85%) | |
| Q8NGV6 | Seven transmembrane | 1 . . . 309 | 241/309 | e-139 |
| | helix receptor - | 1 . . . 309 | (77%) | |
| | Home sapiens | | 267/309 | |
| | (Human), 309 aa. | (85%) | | |

PFam analysis indicates that the NOV5a protein contains the domains shown in the Table 5F.

TABLE 5F
Domain Analysis of NOV5a

| | | Identities/ | |
|---|---|---|---|
| | NOV5a | Similarities for | |
| Pfam Domain | Match Region | the Matched Region | Expect Value |
| 7tm_1 | 41 . . . 290 | 43/276 (16%) | 1.2e-19 |
| | | 169/276 (61%) | |

### Example 6

The NOV6 clone was analyzed, and the nucleotide and encoded polypeptide sequences are shown in Table 6A.

TABLE 6A
NOV6 Sequence Analysis

| | | |
|---|---|---|
| NOV6a, CG187667-01 | SEQ ID NO: 79 | 2680 bp |

| | | |
|---|---|---|
| DNA Sequence | ORF Start: ATG at 873 | ORF Stop: TAG at 2484 |

GGGCAGGTGTTAAGCGATAAAGGGGGGCAGAACAAGTCTTTTATCCGGCCATTTAATTGGTCACCTTA
AGAAAGAATCCTATCTTCCCACCGCAGCTGAGAAGACACTTCTAAGAGGTACAGCTAAAAGGTGGAGG
ACTGATAGACAACTTAGAGGTTGGTTTCGGGAACGATAAAACAAGTAACGGGCTGCCCGGCCTGCGCC
GCGGAGTCCCGAGGAGCCTGCGCTGTGCTCCTCTCGCGGTGTCTCGTCATCTCCGGGAAGACTCGGCG
CCTGGGTCCGCGCTCTCTGGGTAAGCTTTCCGGGAAGCTTTCCCGGGAGCTCGCTGGTCCTGGCCCCA
GAAGCCTGCGGACCCGCCCAGGGAGGATAAGCAGCTGAAAGACCGCGCGGTGCCGCTCCGAGGCCCCG
GGACGTGGGCCCATGGTCGGCCTGGCGCCACCTTTCCGGGGGAAGCCACGCGCACCAGGCATCGCACG
CGGCTCTGCACCCGCGCCGCCGGACCTGAAACCCGGCGGAGGGCACACGGGGCTGCCGCTGCGGGCCC
CGGACCAACCCATGCTTACTCCGGAGCCTGTACCGGCGCCGACGGGTCGGACCTCCCTGCGCGGTGTC
GCCCAGCGGGTTCGTGCGAAAGGCGGGGCCGACTACACGCGGTGCCGCGCCCTGAGACCGTTTATCTG
CAGTCAACGCAGCCTCCCGGCTCAGCCTGGGAAGATGCGCGAATCGGGAACCCCAGAGCGCGGTGGCT
AGACCGGGCTCCGCCGCCTCCCCCACAGCCCCTTTCCTAATCGTTCAGACGGAGCCTGGTCGACTTCG
CCGGAGACTGCCAGATCTCGTTCCTCTTCCCTGTGTCATCTTCTTAATTATAAATAATGGGGGATGAA
GATAAAAGAATTACATATGAAGATTCAGAACCATCCACAGGAATGAATTACACGCCCTCCATGCATCA
AGAAGCACAGGAGGAGACAGTTATGAAGCTCAAAGGTATAGATGCAAATGAACCAACAGAAGGAAGTA
TTCTTTTGAAAAGCAGTGAAAAAAAGCTACAAGAAACACCAACTGAAGCAAATCACGTACAAAGACTG
AGACAAATGCTGGCTTGCCCTCCACATGGTTTACTGGACAGGGTCATAACAAATGTTACCATCATTGT
TCTTCTGTGGGCTGTAGTTTGGTCAATTACTGGCAGTGAATGTCTTCCTGGAGGAAACCTATTTGGAA
TTATAATCCTATTCTATTGTGCCATCATTGGTGGTAAACTTTTGGGGCTTATTAAGTTACCTACATTG
CCTCCACTGCCTTCTCTTCTTGGCATGCTGCTTGCAGGGTTTCTCATCAGAAATATCCCAGTCATCAA
CGATAATGTGCAGATCAAGCACAAGTGGTCTTCCTCTTTGAGAAGCATAGCCCTGTCTATCATTCTGG
TTCGTGCTGGCCTTGGTCTGGATTCAAAGGCCCTGAAGAAGTTAAAGGGCGTTTGTGTAAGACTGTCC
ATGGGTCCCTGTATTGTGGAGGCGTGCACATCTGCTCTTCTTGCCCATTACCTGCTGGGTTTACCATG
GCAATGGGGATTTATACTGGGTTTTGTTTTAGGTGCTGTATCTCCAGCTGTTGTGGTGCCTTCAATGC
TCCTTTTGCAGGGAGGAGGCTATGGTGTTGAGAAGGGTGTCCCAACCTTGCTCATGGCAGCTGGCAGC
TTCGATGACATTCTGGCCATCACTGGCTTCAACACATGCTTGGGCATAGCCTTTTCCACAGGCTCTAC
TGTCTTTAATGTCCTCAGAGGAGTTTTGGAGGTGGTAATTGGTGTGGCAACTGGATCTGTTCTTGGAT
TTTTCATTCAGTACTTTCCAAGCCGTGACCAGGACAAACTTGTGTGTAAGAGAACATTCCTTGTGTTG
GGGTTGTCTGTGCTAGCTGTGTTCAGCAGTGTGCATTTTGGTTTCCCTGGATCAGGAGGACTGTGCAC
GTTGGTCATGGCTTTCCTTGCAGGCATGGGATGGACCAGCGAAAAGGCAGAGGTTGAAAAGATAATTG
CAGTTGCCTGGGACATTTTTCAGCCCCTTCTTTTTGGACTAATTGGAGCAGAGGTATCTATTGCATCT
CTCAGACCAGAAACTGTAGGCCTTTGTGTTGCCACCGTAGGCATTGCAGTATTGATACGAATTTTGAC
TACATTTCTGATGGTGTGTTTTGCTGGTTTTAACTTAAAAGAAAAGATATTTATTTCTTTTGCATGGC
TTCCAAAGGCCACAGTTCAGGCTGCAATAGGATCTGTGGCTTTGGACACAGCAAGGTCACATGGAGAG
AAACAATTAGAAGACTATGGAATGGATGTGTTGACAGTGGCATTTTTGTCCATCCTCATCACAGCCCC
AATTGGAAGTCTGCTTATTGGTTTACTGGGCCCCAGGCTTCTGCAGAAAGTTGAACATCAAAATAAAG
ATGAAGAAGTTCAAGGAGAGACTTCTGTGCAAGTTTAGGAAGCGCGGATTCTATTACTGGAAACTTTG
GGACTGAAAGGCCAAAGCTTCTGGGCCCACCATCAACGCAGCTCCGCTTTCATTTCTTTCACATACAA
CTTTCCACATAAGATTTCATGCGGAAAAAAAAAAAAAACTCACAAAGGTTTTATACTGATAACAGTAT
ATTAAGTGTTTACTTTGTACACAGCGTC

NOV6a, CG187667-01

| | | | |
|---|---|---|---|
| Protein Sequence | SEQ ID NO: 80 | 537 aa | MW at 57563.3kD |

MGDEDKRITYEDSEPSTGMNYTPSMHQEAQEETVMKLKGIDANEPTEGSILLKSSEKKLQETPTEANH
VQRLRQMLACPPHGLLDRVITNVTIIVLLWAVVWSITGSECLPGGNLFGIIILFYCAIIGGKLLGLIK
LPTLPPLPSLLGMLLAGFLIRNIPVINDNVQIKHKWSSSLRSIALSIILVRAGLGLDSKALKKLKGVC
VRLSMGPCIVEACTSALLAHYLLGLPWQWGFILGFVLGAVSPAVVVPSMLLLQGGGYGVEKGVPTLLM
AAGSFDDILAITGFNTCLGIAFSTGSTVFNVLRGVLEVVIGVATGSVLGFFIQYFPSRDQDKLVCKRT
FLVLGLSVLAVFSSVHFGFPGSGGLCTLVMAFLAGMGWTSEKAEVEKIIAVAWDIFQPLLFGLIGAEV
SIASLRPETVGLCVATVGIAVLIRILTTFLMVCFAGFNLKEKIFISFAWLPKATVQAAIGSVALDTAR
SHGEKQLEDYGMDVLTVAFLSILITAPIGSLLIGLLGPRLLQKVEHQNKDEEVQGETSVQV

| | | |
|---|---|---|
| NOV6b, CG187667-02 | SEQ ID NO: 81 | 2285 bp |

| | | |
|---|---|---|
| DNA Sequence | ORF Start: ATG at 873 | ORF Stop: TAA at 2235 |

GGGCAGGTGTTAAGCGATAAAGGGGGGCAGAACAAGTCTTTTATCCGGCCATTTAATTGGTCACCTTA
AGAAAGAATCCTATCTTCCCACCGCAGCTGAGAAGACACTTCTAAGAGGTACAGCTAAAAGGTGGAGG
ACTGATAGACAACTTAGAGGTTGGTTTCGGGAACGATAAAACAAGTAACGGGCTGCCCGGCCTGCGCC
GCGGAGTCCCGAGGAGCCTGCGCTGTGCTCCTCTCGCGGTGTCTCGTCATCTCCGGGAAGACTCGGCG
CCTGGGTCCGCGCTCTCTGGGTAAGCTTTCCGGGAAGCTTTCCCGGGAGCTCGCTGGTCCTGGCCCCA
GAAGCCTGCGGACCCGCCCAGGGAGGATAAGCAGCTGAAAGACCGCGCGGTGCCGCTCCGAGGCCCCG
GGACGTGGGCCCATGGTCGGCCTGGCGCCACCTTTCCGGGGGAAGCCACGCGCACCAGGCATCGCACG
CGGCTCTGCACCCGCGCCGCCGGACCTGAAACCCGGCGGAGGGCACACGGGGCTGCCGCTGCGGGCCC
CGGACCAACCCATGCTTACTCCGGAGCCTGTACCGGCGCCGACGGGTCGGACCTCCCTGCGCGGTGTC
GCCCAGCGGGTTCGTGCGAAAGGCGGGGCCGACTACACGCGGTGCCGCGCCCTGAGACCGTTTATCTG
CAGTCAACGCAGCCTCCCGGCTCAGCCTGGGAAGATGCGCGAATCGGGAACCCCAGAGCGCGGTGGCT
AGACCGGGCTCCGCCGCCTCCCCCACAGCCCCTTTCCTAATCGTTCAGACGGAGCCTGGTCGACTTCG
CCGGAGACTGCCIGATCTCGTTCCTCTTCCCTGTGTCATCTTCTTAATTATAAATAATGGGGGATGAA
GATAAAAGAATTACATATGAAGATTCAGAACCATCCACAGGAATGAATTACACGCCCTCCATGCATCA
AGAAGCACAGGAGGAGACAGTTATGAAGCTCAAAGGTATAGATGCAAATGAACCAACAGAAGGAAGTA
TTCTTTTGAAAAGCAGTGAAAAAAAGCTACAAGAAACACCAACTGAAGCAAATCACGTACAAAGACTG
AGACAAATGCTGGCTTGCCCTCCACATGGTTTACTGGACAGGGTCATAACAAATGTTACCATCATTGT
TCTTCTGTGGGCTGTAGTTTGGTCAATTACTGGCAGTGAATGTCTTCCTGGAGGAAACCTATTTGGAA
TTATAATCCTATTCTATTGTGCCATCATTGGTGGTAAACTTTTGGGGCTTATTAAGTTACCTACATTG
CCTCCACTGCCTTCTCTTCTTGGCATGCTGCTTGCAGGGTTTCTCATCAGAAATATCCCAGTCATCAA
CGATAATGTGCAGATCAAGCACAAGTGGTCTTCCTCTTTGAGAAGCATAGCCCTGTCTATCATTCTGG
TTCGTGCTGGCCTTGGTCTGGATTCAAAGGCCCTGAAGAAGTTAAAGGGCGTTTGTGTAAGACTGTCC
ATGGGTCCCTGTATTGTGGAGGCGTGCACATCTGCTCTTCTTGCCCATTACCTGCTGGGTTTACCATG
GCAATGGGGATTTATACTGGGTTTTGTTTTAGGTGCTGTATCTCCAGCTGTTGTGGTGCCTTCAATGC
TCCTTTTGCAGGGAGGAGGCTATGGTGTTGAGAAGGGTGTCCCAACCTTGCTCATGGCAGCTGGCAGC
TTCGATGACATTCTGGCCATCACTGGCTTCAACACATGCTTGGGCATAGCCTTTTCCACAGGCTCTAC
TGTCTTTAATGTCCTCAGAGGAGTTTTGGAGGTGGTAATTGGTGTGGCAACTGGATCTGTTCTTGGAT
TTTTCATTCAGTACTTTCCAAGCCGTGACCAGGACAAACTTGTGTGTAAGAGAACATTCCTTGTGTTG
GGGTTGTCTGTGCTAGCTGTGTTCAGCAGTGTGCATTTTGGTTTCCCTGGATCAGGAGGACTGTGCAC
GTTGGTCATGGCTTTCCTTGCAGGCATGGGATGGACCAGCGAAAAGGCAGAGGTTGAAAAGATAATTG
CAGTTGCCTGGGACATTTTTCAGCCCCTTCTTTTTGGACTAATTGGAGCAGAGGTATCTATTGCATCT
CTCAGACCAGAAACTGTAGGAAGCGCGGATTCTATTACTGGAAACTTTGGGACTGAAAGGCCAAAGCT
TCTGGGCCCACCATCAACGCAGCTCCGCTTTCATTTCTTTCACATACAACTTTCCACATAAGATTTCA
TGCGGAAAAAAAAAAAAAACTCACAAAGGTTTTATACTGAT

NOV6b, CG187667-02

| | | | |
|---|---|---|---|
| Protein Sequence | SEQ ID NO: 82 | 454 aa | MW at 48709.8kD |

MGDEDKRITYEDSEPSTGMNYTPSMHQEAQEETVMKLKGIDANEPTEGSILLKSSEKKLQETPTEANH
VQRLRQMLACPPHGLLDRVITNVTIIVLLWAVVWSITGSECLPGGNLFGIIILFYCAIIGGKLLGLIK
LPTLPPLPSLLGMLLAGFLIRNIPVINDNVQIKHKWSSSLRSIALSIILVRAGLGLDSKALKKLKGVC
VRLSMGPCIVEACTSALLAHYLLGLPWQWGFILGFVLGAVSPAVVVPSMLLLQGGGYGVEKGVPTLLM
AAGSFDDILAITGFNTCLGIAFSTGSTVFNVLRGVLEVVIGVATGSVLGFFIQYFPSRDQDKLVCKRT
FLVLGLSVLAVFSSVHFGFPGSGGLCTLVMAFLAGMGWTSEKAEVEKIIAVAWDIFQPLLFGLIGAEV
SIASLRPETVGSADSITGNFGTERPKILGPPSTQLRFHFFHIQLST

A ClustalW comparison of the above protein sequences yields the following sequence alignment shown in Table 6B.

TABLE 6B
Comparison of the NOV6 protein sequences.

| | |
|---|---|
| NOV6a | MGDEDKRITYEDSEPSTGMNYTPSMHQEAQEETVMKLKGIDANEPTEGSILLKSSEKKLQ |
| NOV6b | MGDEDKRITYEDSEPSTGMNYTPSMHQEAQEETVMKLKGIDANEPTEGSILLKSSEKKLQ |
| NOV6a | ETPTEANHVQRLRQMLACPPHGLLDRVITNVTIIVLLWAVVWSITGSECLPGGNLFGIII |
| NOV6b | ETPTEANHVQRLRQMLACPPHGLLDRVITNVTIIVLLWAVVWSITGSECLPGGNLFGIII |
| NOV6a | LFYCAIIGGKLLGLIKLPTLPPLPSLLGMLLAGFLIRNIPVINDNVQIKHKWSSSLRSIA |
| NOV6b | LFYCAIIGGKLLGLIKLPTLPPLPSLLGMLLAGFLIRNIPVINDNVQIKHKWSSSLRSIA |
| NOV6a | LSIILVRAGLGLDSKALKKLKGVCVRLSMGPCIVEACTSALLAHYLLGLPWQWGFILGFV |
| NOV6b | LSIILVRAGLGLDSKALKKLKGVCVRLSMGPCIVEACTSALLAHYLLGLPWQWGFILGFV |
| NOV6a | LGAVSPAVVVPSMLLLQGGGYGVEKGVPTLLMAAGSFDDILAITGFNTCLGIAFSTGSTV |
| NOV6b | LGAVSPAVVVPSMLLLQGGGYGVEKGVPTLLMAAGSFDDILAITGFNTCLGIAFSTGSTV |
| NOV6a | FNVLRGVLEVVIGVATGSVLGFFIQYFPSRDQDKLVCKRTFLVLGLSVLAVFSSVHFGFP |
| NOV6b | FNVLRGVLEVVIGVATGSVLGFFIQYFPSRDQDKLVCKRTFLVLGLSVLAVFSSVHFGFP |
| NOV6a | GSGGLCTLVMAFLAGMGWTSEKAEVEKIIAVAWDIFQPLLFGLIGAEVSIASLRPETVGL |
| NOV6b | GSGGLCTLVMAFLAGMGWTSEKAEVEKIIAVAWDIFQPLLFGLIGAEVSIASLRPETVGS |
| NOV6a | CVATVGIAVLIRILTTFLMVCFAGFNLKEKIFISFAWLPKATVQAAIGSVALDTARSHGE |
| NOV6b | ADSITGNFGTERPK--LLG-----------P-------PSTQLRFHFFHIQLST------ |
| NOV6a | KQLEDYGMDVLTVAFLSILITAPIGSLLIGLLGPRLLQKVEHQNKDEEVQGETSVQV |
| NOV6b | --------------------------------------------------------- |
| NOV6a | (SEQ ID NO: 80) |
| NOV6b | (SEQ ID NO: 82) |

Further analysis of the NOV6a protein yielded the following properties shown in Table 6C.

TABLE 6C
Protein Sequence Properties NOV6a

```
Signa1P analysis: No Known Signal Sequence Indicated
PSORT II analysis:
PSG: a new signal peptide prediction method
    N-region: length 11; pos. chg 2; neg. chg 4
    H-region: length 0; peak value 0.00
    PSG score: −4.40
GvH: von Heijne's method for signal seq. recognition
    GvH score (threshold: −2.1): −13.82
    possible cleavage site: between 48 and 49
>>> Seems to have no N-terminal signal peptide
ALOM: Klein et al's method for TM region allocation
    Init position for calculation: 1
    Tentative number of TMS(s) for the threshold 0.5: 10
    INTEGRAL Likelihood = −5.20 Transmembrane 88-104
    INTEGRAL Likelihood = −6.10 Transmembrane 118-134
    INTEGRAL Likelihood = −3.13 Transmembrane 146-162
    INTEGRAL Likelihood = −1.06 Transmembrane 176-192
    INTEGRAL Likelihood = −6.26 Transmembrane 234-250
    INTEGRAL Likelihood = −4.09 Transmembrane 308-324
    INTEGRAL Likelihood = −4.94 Transmembrane 341-357
    INTEGRAL Likelihood = −3.19 Transmembrane 388-404
    INTEGRAL Likelihood = −7.91 Transmembrane 418-434
    INTEGRAL Likelihood = −6.69 Transmembrane 493-509
    PERIPHERAL Likelihood = 0.63 (at 213)
    ALOM score: −7.91(number of TMSs: 10)
MTOP: Prediction of membrane topology (Hartmann et al.)
    Center position for calculation: 95
    Charge difference: −2.5 C(−1.0) − N( 1.5)
    N >= C: N-terminal side will be inside
>>> membrane topology: type 3a
MITDISC: discrimination of mitochondrial targeting seq
    R content: 0 Hyd Moment(75): 9.18
    Hyd Moment(95): 7.68G content: 1
    DIE content: 2 S/T content: 0
    Score: −6.91
Gavel: prediction of cleavage sites for mitochondrial preseq
    cleavage site motif not found
NUCDISC: discrimination of nuclear localization signals
    pat4: none
    pat7: none
    bipartite: none
    content of basic residues: 7.4%
    NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 94.1
COIL: Lupas's algorithm to detect coiled-coil regions
    total: 0 residues
--------------------------
Final Results (k = 9/23)
    66.7%: endoplasmic reticulum
    11.1%: nuclear
    11.1%: vesicles of secretory system
    11.1%: mitochondrial
prediction for CG187667-01 is end (k=9)
```

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| ABU11723 | Human MDDT polypeptide SEQ | 1 . . . 537 | 537/537 (100%) | 0.0 |
| | ID 670 - Homo sapiens, 538 aa. | 2 . . . 538 | 537/537 (100%) | |
| | [WO200279449-A2, 10-OCT- | | | |
| | 2002] | | | |
| ABP69843 | Human polypeptide SEQ ID NO | 1 . . . 537 | 526/537 (97%) | 0.0 |
| | 1890 - Homo sapiens, 537 aa. | 1 . . . 537 | 530/537 (97%) | |
| | [WO200270539-A2, 12-SEP- | | | |
| | 2002] | | | |
| AAY94918 | Human secreted protein clone | 100 . . . 464 | 231/365 (63%) | e-137 |
| | dd504_18 protein sequence SEQ | 1 . . . 365 | 290/365 (79%) | |
| | ID NO:42 - Homo sapiens, 396 | | | |
| | aa. [WO200009552-A1, 24-FEB- | | | |
| | 2000] | | | |
| AAU01672 | Human secreted protein encoded | 153 . . . 519 | 224/367 (61%) | e-130 |
| | by gene #13 - Homo sapiens, 370 | 1 . . . 366 | 287/367 (78%) | |
| | aa. [WO200123409-A2, 05-APR- | | | |
| | 2001] | | | |
| AAU01642 | Human secreted protein | 214 . . . 519 | 185/306 (60%) | e-106 |
| | immunogenic epitope encoded by | 1 . . . 305 | 236/306 (76%) | |
| | gene #2 - Homo sapiens, 323 aa. | | | |
| | [WO200123409-A2, 05-APR- | | | |
| | 2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| AAH47447 | LOC133308 protein - Homo | 19 . . . 537 | 519/519(100%) | 0.0 |
| | sapiens (Human), 519 aa. | 1 . . . 519 | 519/519 (100%) | |
| Q96D95 | Hypothetical protein - Homo | 101 . . . 426 | 320/326 (98%) | 0.0 |
| | sapiens (Human), 354 aa | 1 . . . 326 | 321/326 (98%) | |
| | (fragment). | | | |
| Q8C0X2 | Hypothetical glutainic acid-rich | 53 . . . 521 | 263/470 (55%) | e-159 |
| | region/Na+/H+ exchanger | 83 . . . 551 | 359/470 (75%) | |
| | containing protein - Mus | | | |
| | musculus (Mouse), 565 aa. | | | |
| Q95JS4 | Hypothetical 55.5 kDa protein - | 73 . . . 519 | 261/447 (58%) | e-157 |
| | Macaca fascicularis (Crab eating | 53 . . . 498 | 347/447 (77%) | |
| | macaque) (Gynomolgus | | | |
| | monkey), 511 aa. | | | |
| Q8CDX4 | Hypothetical Na+/H+ exchanger | 253 . . . 537 | 247/285 (86%) | e-138 |
| | containing protein - Mus | 1 . . . 284 | 264/285 (91%) | |
| | musculus (Mouse), 295 aa. | | | |

PFam analysis indicates that the NOV6a protein contains the domains shown in the Table 6F.

TABLE 6F
Domain Analysis of NOV6a

| | | Identities/ | |
|---|---|---|---|
| | NOV6a | Similarities for | |
| Pfam Domain | Match Region | the Matched Region | Expect Value |
| LrgA | 114 . . . 199 | 26/114 (23%) | 0.7 |
| | | 60/114 (53%) | |
| Na_H_{—} | 119 . . . 520 | 85/470 (18%) | 1.3e-16 |
| Exchanger | | 286/470 (61%) | |

### Example 7

The NOV7 clone was analyzed, and the nucleotide and encoded polypeptide sequences are shown in Table 7A.

TABLE 7A
NOV7 Sequence Analysis

| | | |
|---|---|---|
| NOV7a, CG187676-01 | SEQ ID NO: 83 | 2474 bp |

| | | |
|---|---|---|
| DNA Sequence | ORF Start: ATG at 5 | ORF Stop: TGA at 2270 |

AAAAATGAAAAAACAGAGGAAAATTCTATGGAGGAAAGGAATCCACTTAGCCTTTTCTGAGAAATGGA
ATACTGGGTTTGGAGGCTTTAAGAAGTTTTATTTTCACCAACACTTGTGCATTCTGAAAGCTAAGCTG
GGAAGGCCAGTTACTTGGAATAGACAGTTGAGACATTTCCAGGGTAGAAAGAAAGCTCTTCAAATCCA
GAAAACGTGGATCAAGGATGAACCCCTTTGTGCTAAGACCAAGTTCAATGTGGCTACTCAAAATGTTA
GTACTTTGTCCTCTAAAGTGAAAAGAAAGGACGCTAAACACTTCATTTCCTCCTCAAAGACTCTCCTG
AGACTCCAAGCAGAGAAGCTGTTGTCATCAGCAAAGAATTCTGACCATGAATACTGCAGAGAGAAAAA
TCTCTTGAAGGCAGTTACTGACTTTCCATCAAATAGTGCTTTAGGTCAGGCCAATGGTCACAGACCTA
GGACAGACCCACAACCTTCTGACTTTCCCATGAAGTTCAATGGGGAGAGCCAAAGTCCAGGTGAGAGT
GGCACGATTGTGGTCACCTTGAACAACCATAAGAGAAAGGGCTTTTGTTACGGCTGCTGCCAAGGGCC
GGAGCACCACAGGAATGGGGGACCCTTGATTCCAAAAAAGTTCCAACTTAACCAACATAGAAGGATAA
AATTATCTCCTCTTATGATGTATGAGAAATTATCCATGATTAGATTTCGGTACAGGATTCTCAGATCC
CAGCACTTCAGAACCAAAAGCAAGGTTTGCAAGCTAAGAAAAGCCCAGCGAAGCTGGGTACAGAAAGT
CACTGGGGACCATCAAGAGACCCGTAGGGAGAACGGTGAGGGTGGCAGTTGCAGCCCATTTCCTTCCC
CAGAACCTAAAGACCCTTCTTGTCGGCATCAGCCGTACTTTCCAGATATGGACAGCAGTGCTGTGGTG
AAGGGGACGAACTCTCATGTGCCTGATTGCCACACTAAAGGAAGCTCTTTCTTGGGCAAGGAGCTTAG
TTTAGACGAAGCATTCCCTGACCAACAGAATGGCAGTGCCACAAACGCCTGGGACCAGTCATCCTGTT
CTTCTCCTAAGTGGGAGTGTACAGAGCTGATTCATGACATCCCCTTACCAGAACATCGTTCTAATACC
ATGTTCATTTCAGAAACTGAAAGAGAAATTATGACTCTGGGTCAGGAAAATCAGACAAGTTCTGTCAG
TGATGACAGAGTAAAACTGTCAGTGTCTGGAGCAGATACATCTGTGAGTAGCGTAGATGGGCCTGTGT
CCCAAAAGGCTGTTCAAAATGAGAACTCATACCAGATGGAGGAGGATGGATCTCTCAAGCAGAGCATT
CTTAGTTCTGAGTTGCTGGACCACCCTTACTGTAAAAGTCCACTGGAGGCTCCCTTGGTGTGCAGTGG
ACTCAAACTAGAAAATCAAGTAGGAGGTGGAAAGAACAGTCAGAAAGCCTCTCCAGTGGATGATGAAC
AGCTGTCAGTCTGTCTTTCTGGATTCCTAGATGAGGTTATGAAGAAGTATGGCAGTTTGGTTCCACTC
AGTGAAAAAGAAGTCCTTGGAAGATTAAAAGATGTCTTTAATGAAGACTTTTCTAATAGAAAACCATT
TATCAATAGGGAAATAACAAACTATCGGGCCAGACATCAAAAATGTAACTTCCGTATCTTCTATAATA
AACACATGCTGGATATGGACGACCTGGCGACTCTGGATGGTCAGAACTGGCTGAATGACCAGGTCATT
AATATGTATGGTGAGCTGATAATGGATGCAGTCCCGGACAAAGTTCACTTCTTCAACAGCTTTTTTCA
TAGACAGCTGGTAACCAAAGGATATAATGGAGTAAAAAGATGGACTAAAAAGGTGGATTTGTTTAAAA
AGAGTCTTCTGTTGATTCCTATTCACCTGGAAGTCCACTGGTCTCTCATTACTGTGACACTCTCTAAT
CGAATTATTTCATTTTATGATTCCCAAGGCATTCATTTTAAGTTTTGTGTAGAGAATATAAGAAAGTA
TTTGCTGACTGAAGCCAGAGAAAAAAATAGACCTGAATTTCTTCAGGGTTGGCAGACTGCTGTTACGA
AGTGTATTCCACAACAGAAAAACGACAGTGACTGTGGAGTCTTTGTGCTCCAGTACTGCAAGTGCCTC
GCCTTAGAGCAGCCTTTCCAGTTTTCACAAGAAGACATGCCCCGAGTGCGGAAGAGGATTTACAAGGA
GCTATGTGAGTGCCGGCTCATGGACTGAAACTCAGCAGGGACTCTGGGAAGTCTGACCAAGTTGGAGC
AGATGGTTTGTTACTTGAATCTCCAAACACTTAGTTGAATTTTTACAGATATTTCAGATCAGTGGTGT
TGGGCCACTATTGTTACCTCAAATTTATTTTTTGCCCTTATTCATTTCTCCAGCTACCATGTACTATT
GTTTAATGTTCAGTTTGGTTTCAAAA

NOV7a, CG187676-01

| | | | |
|---|---|---|---|
| Protein Sequence | SEQ ID NO: 84 | 755 aa | MW at 86692.3kD |

MKKQRKILWRKGIHLAFSEKWNTGFGGFKKFYFHQHLCILKAKLGRPVTWNRQLRHFQGRKKALQIQK
TWIKDEPLCAKTKFNVATQNVSTLSSKVKRKDAKHFISSSKTLLRLQAEKLLSSAKNSDHEYCREKNL
LKAVTDFPSNSALGQANGHRPRTDPQPSDFPMKFNGESQSPGESGTIVVTLNNHKRKGFCYGCCQGPE
HHRNGGPLIPKKFQLNQHRRIKLSPLMMYEKLSMIRFRYRILRSQHFRTKSKVCKLRKAQRSWVQKVT
GDHQETRRENGEGGSCSPFPSPEPKDPSCRHQPYFPDMDSSAVVKGTNSHVPDCHTKGSSFLGKELSL
DEAFPDQQNGSATNAWDQSSCSSPKWECTELIHDIPLPEHRSNTMFISETEREIMTLGQENQTSSVSD
DRVKLSVSGADTSVSSVDGPVSQKAVQNENSYQMEEDGSLKQSILSSELLDHPYCKSPLEAPLVCSGL
KLENQVGGGKNSQKASPVDDEQLSVCLSGFLDEVMKKYGSLVPLSEKEVLGRLKDVFNEDFSNRKPFI
NREITNYRARHQKCNFRIFYNKHMLDMDDLATLDGQNWLNDQVINNYGELIMDAVPDKVHFFNSFFHR
QLVTKGYNGVKRWTKKVDLFKKSLLLIPIHLEVHWSLITVTLSNRIISFYDSQGIHFKFCVENIRKYL
LTEAREKNRPEFLQGWQTAVTKCIPQQKNDSDCGVFVLQYCKCLALEQPFQFSQEDMPRVRKRIYKEL
CECRLMD

Further analysis of the NOV7a protein yielded the following properties shown in Table 7B.

TABLE 7B
Protein Sequence Properties NOV7a

```
Signa1P analysis: No Known Signal Sequence Indicated
PSORT II analysis:
PSG: a new signal peptide prediction method
    N-region: length 11; pos. chg 6; neg. chg 0
    H-region: length 7; peak value 0.84
    PSG score: −3.56
GvH: von Heijne's method for signal seq. recognition
    GvH score (threshold: −2.1): −10.14
    possible cleavage site: between 18 and 19
>>> Seems to have no N-terminal signal peptide
ALOM: Klein et al's method for TM region allocation
    Init position for calculation: 1
    Tentative number of TMS(s) for the threshold 0.5: 1
    Number of TMS(s) for threshold 0.5: 0
    PERIPHERAL Likelihood = 6.31 (at 710)
    ALOM score: −0.06 (number of TMSs: 0)
MITDISC: discrimination of mitochondrial targeting seq
    R content: 2 Hyd Moment(75): 3.21
    Hyd Moment(95) : 10.20G content: 1
    D/E content: 1 S/T content: 1
    Score: −3.27
Gavel: prediction of cleavage sites for mitochondrial preseq
    R-2 motif at 70 GRK|KA
NUCDISC: discrimination of nuclear localization signals
    pat4: HKRK (3) at 190
    pat7: PRVRKRI (5) at 738
    bipartite: none
    content of basic residues: 15.1%
    NLS Score: 0.15
KDEL: ER retention motif in the C-terminus: none
ER Membrane Retention Signals:
    XXRR-like motif in the N-terminus: KKQR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: nuclear
    Reliability: 89
COIL: Lupas's algorithm to detect coiled-coil regions
    total: 0 residues
------------------------------
Final Results (k = 9/23)
    65.2%: nuclear
    26.1%: mitochondrial
    4.3%: cytoplasmic
    4.3%: peroxisomal
>> prediction for CG187676-01 is nuc (k=23)
```

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| ABJ26650 | Human protein modification + | 1 . . . 755 | 755/755 (100%) | 0.0 |
| | maintenance molecule protein | 1 . . . 755 | 755/755 (100%) | |
| | SEQ ID No 4 - Homo sapiens, | | | |
| | 755 aa. [WO2003000844-A2, 03- | | | |
| | JAN-2003] | | | |
| AAB71412 | Human HIPHUM106 splice | 1 . . . 755 | 754/755 (99%) | 0.0 |
| | variant SEQ ID 4 - Homo | 1 . . . 755 | 755/755 (99%) | |
| | sapiens, 755 aa. [GB2371801-A, | | | |
| | 07-AUG-2002] | | | |
| AAB71411 | Human HIPIIUM106 protein | 1 . . . 755 | 727/755 (96%) | 0.0 |
| | SEQ ID 2 - Homo sapiens, 727 | 1 . . . 727 | 727/755 (96%) | |
| | aa. [GB2371801-A, 07-AUG- | | | |
| | 2002] | | | |
| ABP69066 | Human polypeptide SEQ ID NO | 310 . . . 752 | 413/443 (93%) | 0.0 |
| | 1113 - Homo sapiens, 419 aa. | 1 . . . 415 | 413/443 (93%) | |
| | [WO200270539-A2, 12-SEP- | | | |
| | 2002] | | | |
| AAM25617 | Human protein sequence SEQ ID | 485 . . . 755 | 267/271 (98%) | e-157 |
| | NO:1132 - Homo sapiens, 270 aa. | 1 . . . 270 | 268/271 (98%) | |
| | [WO200153455-A2 , 26-JUL- | | | |
| | 2001] | | | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q96H10 | Sentrin-specific protease 5 (EC | 1 . . . 755 | 754/755 (99%) | 0.0 |
| | 3.4.22.-) (Sentrin/SUMO-specific | 1 . . . 755 | 754/755 (99%) | |
| | protease SENP5) (Protease | | | |
| | FKSG45) - Homo sapiens | | | |
| | (Human), 755 aa. | | | |
| Q8WP32 | Sentrin-specific protease 5 (EG | 1 . . . 755 | 714/755 (94%) | 0.0 |
| | 3.4.22.-) (Sentrin/SUMO-specific | 1 . . . 755 | 735/755 (96%) | |
| | protease SENP5) (QtsA-16408) - | | | |
| | Macaca fascicularis (Grab eating | | | |
| | macaque) (Cynomolgus monkey), | | | |
| | 755 aa. | | | |
| Q8BXW0 | Hypothetical SUMO/sentrin/Ub11 | 1 . . . 755 | 619/756 (81%) | 0.0 |
| | specific protease containing protein - | 1 . . . 749 | 667/756 (87%) | |
| | Mus musculus (Mouse), 749 aa. | | | |
| Q8BRD5 | Sentrin/SUMO-specific protease - | 485 . . . 753 | 148/274 (54%) | 1e-82 |
| | Mus musculus(Mouse), 568 aa. | 294 . . . 566 | 202/274 (73%) | |
| Q9EP97 | Sentrin-specific protease 3 (EC | 485 . . . 753 | 148/274 (54%) | 1e-82 |
| | 3.4.22.-) (Sentrin/SUMO-specific | 294 . . . 566 | 202/274 (73%) | |
| | protease SENP3) (SUMO-1 | | | |
| | specific protease 3) (Smt3- | | | |
| | specific isopeptidase 1) (Smt3ip1) - | | | |
| | Mus musculus (Mouse), 568 aa. | | | |

PFam analysis indicates that the NOV7a protein contains the domains shown in the Table 7E.

TABLE 7E
Domain Analysis of NOV7a

| | | Identities/ | |
|---|---|---|---|
| | NOV7a | Similarities for | |
| Pfam Domain | Match Region | the Matched Region | Expect Value |
| Peptifase_{—} | 563 . . . 752 | 62/248 (25%) | 7e-43 |
| C48 | | 149/248 (60%) | |

### Example 8

The NOV8 clone was analyzed, and the nucleotide and encoded polypeptide sequences are shown in Table 8A.

TABLE 8A
NOV8 Sequence Analysis

| | | |
|---|---|---|
| NOV8a, CG50235-04 | SEQ ID NO: 85 | 3120 bp |

| | | |
|---|---|---|
| DNA Sequence | ORF Start: ATG at 256 | ORF Stop: TGA at 2719 |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCAGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGGCCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATTCCCACGCCCAGTTTGGGGACAACAACTACCCGAGCGA
GGCCCGCTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGATTCCCTGAT
GATTCGATTCCGCACAGATGACACCATCAACAAGAAAGGCTTTCATGCCCGATACACCAGCACCAAGT
TCCAGGATGCCCTGCACATGAAGAAATAGTGCTGATGTTCTTGAAAGACAGAAACTGAGA

NOV8a, CG50235-04

| | | | |
|---|---|---|---|
| Protein Sequence | SEQ ID NO: 86 | 821 aa | MW at 91404.2kD |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTWNISSTAGHRVKL
SAGSG

| | | |
|---|---|---|
| NOV8b, CG50235-01 | SEQ ID NO: 87 | 3350 bp |

| | | |
|---|---|---|
| DNA Sequence | ORF Start: ATG at 365 | ORF Stop: TAG at 3341 |

CGCCCATTGGCTCCTCAGCCAAGCACGTACACCAAATGTCTGAACCTGCGGTTCCTCTCGTACTGAGC
AGGATTACCATGGCAACAACACATCATCAGTAGGGTAAAACTAACCTGTCTCACGACGGTCTAAACCC
AGGCAGCCTCGGCCGCCGGGCAAGTAGCTCCGAGCGGCTGCTTCCCGGTTGCCTCGAAGAAGACAGGG
GGCGCCGCGCTCCGCTTGCTCCGCGCCTGAGCCATGCCCAGCAGCCCTGTGTAACCACCGAGTCCCGG
CCGGAGCCGACCGACCCAGTGTGCGCCGTCTTTCGGCCGAGCTGAGCTTTCGTGCACGCAACTCCCTC
TGCCCCAGCCGGCCCCGCGCCACCATGCCCCGGGCGACTGCACTTGGGGCCCTGGTGTCACTGCTGCT
GCTGCTGCCGCTGCCTCGCGGCGCCGGGGGACTCGGGGAGCGCCCGGACGCCACCGCAGACTACTCAG
AGCTGGACGGCGAGGAGGGCACGGAGCAGCAGCTGGAGCATTACCACGACCCTTGCAAAGCCGCTGTC
TTTTGGGGAGACATTGCCTTAGATGAAGATGACTTGAAGCTGTTTCACATTGACAAAGCCAGAGACTG
GACCAAGCAGACAGTGGGGGCAACAGGACACAGCACAGGTGGGCTTGAAGAGCAGGCATCTGAGAGCA
GCCCAGACACCACAGCCATGGACACTGGCACCAAGGAAGCTGGAAAGGATGGCCGGGAGAATACCACA
CTCCTGCACAGCCCTGGGACCTTGCATGCCGCAGCCAAGACCTTCTCTCCCCGGGTCCGAAGAGCCAC
AACCTCAAGGACAGAGAGGATATGGCCTGGAGGAGTCATCCCCTACGTCATTGGAGGGAACTTCACTG
GGAGCCAGAGGGCCATTTTTAAGCAGGCCATGAGACACTGGGAGAAGCACACCTGTGTGACCTTCATA
GAAAGGACGGATGAGGAAAGCTTTATTGTATTCAGTTACAGAACCTGTGGCTGTTGCTCCTATGTTGG
GCGCCGAGGAGGAGGCCCACAGGCCATATCCATTGGGAAGAACTGTGACAAGTTTGGCATTGTGGCTC
ACGAGCTGGGCCATGTGGTTGGGTTTTGGCATGAACACACCCGGCCAGACAGAGACCAACATGTCACC
ATCATCAGGGAAAACATCCAGCCAGGTCAGGAGTATAATTTCTTAAAAATGGAAGCTGGGGAAGTGAG
CTCTCTGGGAGAGACATACGACTTTGACAGCATCATGCACTACGCCCGGAACACCTTCTCAAGAGGAG
TTTTCTTAGACACCATCCTTCCCCGTCAAGATGACAATGGCGTCAGGCCAACCATTGGCCAGCGCGTG
CGGCTCAGTCAGGGAGACATAGCTCAAGCCCGGAAGCTGTACAAATGCCCAGCGTGTGGGGAGACCCT
GCAGGACACAACGGGAAACTTTTCTGCACCTGGTTTCCCAAATGGGTACCCATCTTACTCCCACTGCG
TCTGGAGGATCTCGGTCACCCCAGGGGAAAAGATCGTATTAAACTTCACATCCATGGATTTGTTTAAA
AGCCGACTGTGCTGGTATGATTACGTGGAGGTCCGGGATGGTTACTGGAGAAAAGCCCCCCTTTTGGG
CAGGTTTTGTGGCGATAAGATCCCGGAGCCCCTCGTCTCCACGGACAGCCGGCTCTGGGTGGAGTTCC
GCAGCAGCAGCAACATCTTGGGCAAGGGCTTCTTTGCAGCGTACGAAGCTACCTGCGGGGGAGACATG
AACAAAGATGCCGGTCAGATTCAATCTCCCAACTATCCGGATGACTACAGACCTTCCAAGGAATGTGT
CTGGAGGATTACGGTTTCAGAGGGGTTTCACGTGGGACTTACCTTCCAAGCTTTTGAGATTGAAAGGC
ACGACAGCTGTGCATATGACTACCTGGAAGTCCGGGATGGCCCCACGGAAGAGAGTGCCCTGATCGGC
CACTTTTGTGGCTATGAGAAGCCGGAGGATGTGAAATCGAGCTCCAACAGACTGTGGATGAAGTTTGT
GTCCGATGGCTCTATCAATAAAGCGGGCTTTGCAGCCAATTTTTTCAAGGAGGTGGATGAGTGTTCCT
GGCCAGATCACGGCGGGTGCGAACATCGCTGTGTGAACACGCTGGGCAGCTACAAGTGTGCCTGTGAC
CCTGGCTACGAGCTGGCCGCCGATAAGAAGATGTGTGAAGTGGCCTGTGGCGGTTTCATTACCAAGCT
GAATGGAACCATCACCAGCCCTGGGTGGCCGAAGGAGTATCCCACAAACAAAAACTGTGTCTGGCAGG
TGGTGGCCCCCACTCAGTACCGGATCTCCCTTCAGTTTGAAGTGTTTGAACTGGAAGGCAATGACGTC
TGTAAGTACGACTTTGTAGAGGTGCGCAGCGGCCTGTCCCCCGACGCCAAGCTGCACGGCAGGTTCTG
CGGCTCTGAGACGCCGGAAGTCATCACCTCGCAGAGCAACAACATGCGCGTGGAGTTCAAGTCCGACA
ACACCGTCTCCAAGCGCGGCTTCAGGGCCCACTTCTTCTCAGATAAGGACGAGTGTGCCAAGGACAAC
GGCGGGTGTCAGCATGAGTGCGTCAACACCTTCGGGAGCTACCTGTGCAGGTGCAGAAACGGCTACTG
GCTCCACGAGAATGGGCATGACTGCAAAGAGGCTGGCTGTGCACACAAGATCAGCAGTGTGGAGGGGA
CCCTGGCGAGCCCCAACTGGCCTGACAAATACCCCAGCCGGAGGGAGTGTACCTGGAACATCTCTTCG
ACTGCAGGCCACAGAGTGAAACTCACCTTTAATGAGTTTGAGATCGAGCAGCACCAGGAATGTGCCTA
TGACCACCTGGAAATGTATGACGGGCCGGACAGCCTGGCCCCCATTCTGGGCCGTTTCTGCGGCAGCA
AGAAACCAGACCCCACGGTGGCTTCCGGCAGCAAGTGCGGGGGCAGGCTGAAGGCTGAAGTGCAGACC
AAAGAGCTCTATTCCCACGCCCAGTTTGGGGACAACAACTACCCGAGCGAGGCCCGCTGTGACTGGGT
GATCGTGGCAGAGGACGGCTACGGCGTGGAGCTGACATTCCGGACCTTTGAGGTTGAGGAGGAGGCCG
ACTGCGGCTACGACTACATGGAAGCCTACGACGGCTACGACAGCTCAGCGCCCAGGCTCGGCCGCTTC
TGTGGCTCTGGGCCATTAGAAGAAATCTACTCTGCAGGTGATTCCCTGATGATTCGATTCCGCACAGA
TGACACCATCAACAAGAAAGGCTTTCATGCCCGATACACCAGCACCAAGTTCCAGGATGGCCTGCACA
TGAAGAAATAGTGCTGAT

NOV8b, CG50235-01

| | | | |
|---|---|---|---|
| Protein Sequence | SEQ ID NO: 88 | 992 aa | MW at 110925.6kD |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGRENTTLLHSPGTL
HAAAKTFSPRVRRATTSRTERIWPGGVIPYVIGGNFTGSQRAIFKQANRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWNKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPTQYRISLQFEVFELEGNDVCKYDFVEV
RSGLSPDAKLHGRFCGSETPEVITSQSNNNRVEFKSDNTVSKRGFRAHFFSDKDECAKDMGGCQHECV
NTFGSYLCRCRNGYWLHENGHDCKEAGCAHKISSVEGTLASPNWPDKYPSRRECTWNISSTAGHRVKL
TFNEFEIEQHQECAYDHLEMYDGPDSLAPILGRFCGSKKPDPTVASGSKCGGRLKAEVQTKELYSHAQ
FGDNNYPSEARCDWVIVAEDGYGVELTFRTFEVEEEADCGYDYMEAYDGYDSSAPRLGRFCGSGPLEE
IYSAGDSLMIRFRTDDTINKKGFHARYTSTKFQDGLHMKK

| | | |
|---|---|---|
| NOV8c, CG50235-02 | SEQ ID NO: 89 | 5006 bp |

| | | |
|---|---|---|
| DNA Sequence | ORF Start: ATG at 365 | ORF Stop: TAG at 3410 |

CGCCCATTGGCTCCTCAGCCAAGCACGTACACCAAATGTCTGAACCTGCGGTTCCTCTCGTACTGAGC
AGGATTACCATGGCAACAACACATCATCAGTAGGGTAAAACTAACCTGTCTCACGACGGTCTAAACCC
AGGCAGCCTCGGCCGCCGGGCAAGTAGCTCCGAGCGGCTGCTTCCCGGTTGCCTCGAAGAAGACAGGG
GGCGCCGCGCTCCGCTTGCTCCGCGCCTGAGCCATGCCCAGCAGCCCTGTGTAACCACCGAGTCCCGG
CCGGAGCCGACCGACCCAGTGTGCGCCGTCTTTCGGCCGAGCTGAGCTTTCGTGCACGCAACTCCCTC
TGCCCCAGCCGGCCCCGCGCCACCATGCCCCGGGCGACTGCACTTGGGGCCCTGGTGTCACTGCTGCT
GCTGCTGCCGCTGCCTCGCGGCGCCGGGGGACTCGGGGAGCGCCCGGACGCCACCGCAGACTACTCAG
AGCTGGACGGCGAGGAGGGCACGGAGCAGCAGCTGGAGCATTACCACGACCCTTGCAAAGCCGCTGTC
TTTTGGGGAGACATTGCCTTAGATGAAGATGACTTGAAGCTGTTTCACATTGACAAAGCCAGAGACTG
GACCAAGCAGACAGTGGGGGCAACAGGACACAGCACAGGTGGGCTTGAAGAGCAGGCATCTGAGAGCA
GCCCAGACACCACAGCCATGGACACTGGCACCAAGGAAGCTGGAAAGGATGGCCGGGAGAATACCACA
CTCCTGCACAGCCCTGGGACCTTGCATGCCGCAGCCAAGACCTTCTCTCCCCGGGTCCGAAGAGCCAC
AACCTCAAGGACAGAGAGGATATGGCCTGGAGGAGTCATCCCCTACGTCATTGGAGGGAACTTCACTG
GGAGCCAGAGGGCCATTTTTAAGCAGGCCATGAGACACTGGGAGAAGCACACCTGTGTGACCTTCATA
GAAAGGACGGATGAGGAAAGCTTTATTGTATTCAGTTACAGAACCTGTGGCTGTTGCTCCTATGTTGG
GCGCCGAGGAGGAGGCCCACAGGCCATATCCATTGGGAAGAACTGTGACAAGTTTGGCATTGTGGCTC
ACGAGCTGGGCCATGTGGTTGGGTTTTGGCATGAACACACCCGGCCAGACAGAGACCAACATGTCACC
ATCATCAGGGAAAACATCCAGCCAGGTCAGGAGTATAATTTCTTAAAAATGGAAGCTGGGGAAGTGAG
CTCTCTGGGAGAGACATACGACTTTGACAGCATCATGCACTACGCCCGGAACACCTTCTCAAGAGGAG
TTTTCTTAGACACCATCCTTCCCCGTCAAGATGAGATGGCGTCCAGGCCAACCATTGGCCAGCGCGTG
CGGCTCAGTCAGGGAGACATAGCTCAAGCCCGGAAGCTGTACAAATGCCCAGCGTGTGGGGAGACCCT
GCAGGACACAACGGGAAACTTTTCTGCACCTGGTTTCCCAAATGGGTACCCATCTTACTCCCACTGCT
TCTGGAGGATCTCGGTCACCCCAGGGGAAAAGATCGTATTAAACTTCACATCCATGGATTTGTTTAAA
AGCCGACTGTGCTGGTATGATTACGTGGAGGTCCGGGATGGTTACTGGAGAAAAGCCCCCCTTTTGGG
CAGGTTTTGTGGCGATAAGATCCCGGAGCCCCTCGTCTCCACGGACAGCCGGCTCTGGGTGGAGTTCC
GCAGCAGCAGCAACATCTTGGGCAAGGGCTTCTTTGCAGCGTACGAAGCTACCTGCGGAAGAGACATG
AACAAAGATGCCGGTCAGATTCAATCTCCCAACTATCCGGATGACTACAGACCTTCCAAGGAATGTGT
CTGGAGGATTACGGTTTCGGAGGGGTTTCACGTGGGACTTACCTTCCAAGCTTTTGAGATTGAAAGGC
ACGACAGCTGTGCATATGACTACCTGGAAGTCCGGGATGGCCCCACGGAAGAGAGTGCCCTGATCGGC
CACTTTTGTGGCTATGAGAAGCCGGAGGATGTGAAATCGAGCTCCAACAGACTGTGGATGAAGTTTGT
GTCCGATGGCTCTATCAATAAAAGCGGGCTTTGCAGCCAATTTTTTCAAGGAGGTGGATGAGTGTTCT
GGCCAGATCACGGCGGGTGCGAACATCGCTGTGTGAACACGCTGGGCAGCTACAAGTGTGCCTGTGAC
CCTGGCTACGAGCTGGCCGCCGATAAGAAGATGTGTGAAGTGGCCTGTGGCGGTTTCATTACCAAGCT
GAATGGAACCATCACCAGCCCTGGGTGGCCGAAGGAGTATCCCACAAACAAAAACTGTGTCTGGCAGG
TGGTGGCCCCCGCTCAGTACCGGATCTCCCTTCAGTTTGAAGTGTTTGAACTGGAAGGCAATGACGTC
TGTAAGTACGACTTTGTACAGGTGCGCAGCGGCCTGTCCCCCGACGCCAAGCTGCACGGCAGGTTCTG
CGGCTCTGAGACGCCGGAAGTCATCACCTCGCAGAGCAACAACATGCGCGTGGAGTTCAAGTCCGACA
ACACCGTCTCCAAGCGCGGCTTCAGGGCCCACTTCTTCTCAGATAAGGACGAGTGTGCCAAGGACAAC
GGCGGGTGTCAGCATGAGTGCGTCAACACCTTCGGGAGCTACCTGTGCAGGTGCAGAAACGGCTACTG
GCTCCACGAGAATGGGCATGACTGCAAAGAGGCTGGCTGTGCACACAAGATCAGCAGTGTGGAGGGGA
CCCTGGCGAGCCCCAACTGGCCTGACAAATACCCCAGCCGGAGGGAGTGTACCTGGAACATCTCTTCG
ACTGCAGGCCACAGAGTGAAACTCACCTTTAATGAGTTTGAGATCGAGCAGCACCAGGAATGTGCCTA
TGACCACCTGGAAATGTATGACGGGCCGGACAGCCTGGCCCCCATTCTGGGCCGTTTCTGCGGCAGCA
AGAAACCAGACCCCACGGTGGCTTCCGGCAGCAGTATGTTTCTCAGGTTTTATTCGGATGCCTCAGTG
CAGAGGAAAGGCTTCCAGGCAGTGCACAGCACAGAGTGCGGGGGCAGGCTGAAGGCTGAAGTGCAGAC
CAAAGAGCTCTATTCCCACGCCCAGTTTGGGGACAACAACTACCCGAGCGAGGCCCGCTGTGACTGGG
TGATCGTGGCAGAGGACGGCTACGGCGTGGAGCTGACATTCCGGACCTTTGAGGTTGAGGAGGAGGCC
GACTGCGGCTACGACTACATGGAAGCCTACGACGGCTACGACAGCTCAGCGCCCAGGCTCGGCCGCTT
CTGTGGCTCTGGGCCATTAGAAGAAATCTACTCTGCAGGTGATTCCCTGATGATTCGATTCCGCACAG
ATGACACCATCAACAAGAAAGGCTTTCATGCCCGATACACCAGCACCAAGTTCCAGGATGCCCTGCAC
ATGAAGAAATAGTGCTGATGTTCTTGAAAGACAGAAACTGAGAATTTTTTTGTTTTGTTTTGTTTTTA
ACAACAATAGCACCTTGAAAATCTGCCCTAAAACAGTGTACAGTATTTTTCTCAAACAAAAACTCAGA
ATCCAGCCTTAGAGGTATATATTTGAATGAAAGTCTTGTAAGTTTGGCCAACAAGGTGGAGAAAAAAA
TGTTCTTTTGCTTCTGTCTGCAATGTTGTCATTCATGAACTGTTAAAGTGTTAAAGATTAGGATTGGA
GTCACTGACCATTCCGGCTATGCTTCTTCATACCATTCTCCTTGTTGTCCCTTGCTCCTATGTGGCAA
AAGGTCAGCCTTGGGGTTGGCCGTTCCTCTAATCTGGACTTGCTTGCTTGGTGCCAGGTGCGTCTTCT
GTCCATGTTGGGCATAAGGGATGAAAACTTGGCCGAGACTAATGTGTGGCCCACAGCTTTGGCTGGAA
TCATTTTCTTTCTCTCTGCCAGGGACATGTCAACCAAGAAACCTGAAAATATGGATGGATGTCAGGAC
TAAAAAAAGGCATCACAGTGAGCAGTGAGCACAGAGGGAGTTTCGAGTATAAGAATCATTGTCATGAA
GTTAGGAGACCACAAAGCCATTTCTCAGAGTCATTCACTCTCCTTGTCCCTTTGGTTTCCCCCCTTCC
TTAATTGCAGTGGGGGCTAAGGTATCCATTATGAATACAGCAGAACATTTGCTGGCGAGAGTCCTGTC
TGCTGAGAAGACAATATTGTGGCTCGTCCTGATATTTTTTCATTCATTGACTTTGAGAAGACTCCACC
TGTGCTTGGAATTCCATGGGCTTCAAAGAACATTTCTTCTTTTAGCTTTGGAGGCACTTGCCGTGGCA
CACCTGGACTCCTTGACATCCAATTCAAACTGCATTTGCAAAATGTGCAAAGACCTCTTATGAGGGAC
CAATTCAGGTCCCTTATGGGGTGAACACTGTTGAAGACTGGTTAATTATAAGTTATGTAAGAATCATC
GCCTTGTGGAACAAGTCAATCAGTGACTAGCTTCCTGTAGCCAATCAGGTTAAAGAGGGCGTTGGTAA
TTTTGTTCTGATTTAACTAGTATTCAATCACCAACTTGCAAACAGAATTCATAACACTTGGCACTTGT
TCTAGAGAAGTGTAGAGGATGATGTTAACATAATTTTAGCACTTCAAGGTATAATTTAAACAGTGAGG
TAGTTTTGAATGGCATTTCATTAAGGCATCTATGGGCATTATGAGCTAAAAGCTGTGGTATGTTAGCT
TTAAAAGAGTATTTATGTTGGAATAATTTTTAAATAATGTTTACATAACTGTAAGTCCTGTTTGGTTG
TTGTTGGACGCAGGGCGGCACATGAGTGTTTTTGGTTAGAGCCAAGATAGCTCCCATGCACCGGAATT
CCTTTGGGATGAATCAGCATCATTTTAAACAAAGTATATGTAAAAGGTGAAAGGTTATATTTTTTACA
GATCAGAATGTGGCACCAGAGGACTGTGTCTCATTAAAGTGATTGCTGGGAGCAAAAACTAGAATGAT
ACAAAGAAAGGTCAGAGAAATGCATGGGAATATTTTTTCTTT

NOV8c, CG50235-02

| | | | |
|---|---|---|---|
| Protein Sequence | SEQ ID NO: 90 | 1015 aa | MW at 113555.5kD |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTWNISSTAGHRVKL
TFNEFEIEQHQECAYDHLEMYDGPDSLAPILGRFCGSKKPDPTVASGSSMFLRFYSDASVQRKGFQAV
HSTECGGRLKAEVQTKELYSHAQFGDNNYPSEARCDWVIVAEDGYGVELTFRTFEVEEEADCGYDYME
AYDGYDSSAPRLGRFCGSGPLEEIYSAGDSLMIRFRTDDTINKKGFHARYTSTKFQDALHMKK

| | | |
|---|---|---|
| NOV8d, CG50235-03 | SEQ ID NO: 91 | 3146 bp |

| | | |
|---|---|---|
| DNA Sequence | ORF Start: ATG at 227 | ORF Stop: TAG at 3137 |

GCAGCCTCGGCCGCCGGGCAAGTAGCTCCGAGCGGCTGCTTCCCGGTTGCCTCGACGAAGACAGGGGG
CGCCGCGCTCCGCTTGCTCCGCGCCTGAGCCATGCCCAGCAGCCCTGTGTAACCACCGAGTCCCGGCC
GGAGCCGACCGACCCAGTGTGCGCCGTCTTTCGGCCGAGCTGAGCTTTCGTGCACGCAACTCCCTCTG
CCCCAGCCGGCCCCGCGCCACCATGCCCCGGGCGACTGCACTTGGGGCCCTGGTGTCACTGCTGCTGC
TGCTGCCGCTGCCTCGCGGCGCCGGGGGACTCGGGGAGCGCCCGGACGCCACCGCAGACTACTCAGAG
CTGGACGGCGAGGAGGGCACGGAGCAGCAGCTGGAGCATTACCACGACCCTTGCAAAGCCGCTGTCTT
TTGGGGAGACATTGCCTTAGATGAAGATGACTTGAAGCTGTTTCACATTGACAAAGCCAGAGACTGGA
CCAAGCAGACAGTGGGGGCAACAGGACACAGCACAGGTGGGCTTGAAGAGCAGGCATCTGAGAGCAGC
CCAGACACCACAGCCATGGACACTGGCACCAAGGAAGCTGGAAAGGGGAGCCAGAGGGCCATTTTTAA
GCAGGCCATGAGACACTGGGAGAAGCACACCTGTGTGACCTTCATAGAAAGGACGGATGAGGAAAGCT
TTATTGTATTCAGTTACAGAACCTGTGGCTGTTGCTCCTATGTTGGGCGCCGAGGAGGAGGCCCACAG
GCCATATCCATTGGGAAGAACTGTGACAAGTTTGGCATTGTGGCTCACGAGCTGGGCCATGTGGTTGG
GTTTTGGCATGAACACACCCGGCCAGACAGAGACCAACATGTCACCATCATCAGGGAAAACATCCAGC
CAGGTCAGGAGTATAATTTCTTAAAAATGGAAGCTGGGGAAGTGAGCTCTCTGGGAGAGACATACGAC
TTTGACAGCATCATGCACTACGCCCGGAACACCTTCTCAAGAGGAGTTTTTTTAGACACCATCCTTCC
CCGTCAAGATGACAATGGCGTCAGGCCAACCATTGGCCAGCGCGTGCGGCTCAGTCAGGGAGACATAG
CTCAAGCCCGGAAGCTGTACAAATGCCCAGGTCCTACTTGTGCTTTTGTTAGCCAGAAAACATCAATC
TGCTTGCTACACTTCTCACCAACCTGTTCCGAGGGCTTTGGCTGGCAAAGGGCGTGTGGGGAGACCCT
GCAGGACACAACGGGAAACTTTTCTGCACCTGGTTTCCCAAATGGGTACCCATCTTACTCCCACTGCG
TCTGGAGGATCTCGGTCACCCCAGGGGAAAAGATCGTATTAAACTTCACATCCATGGATTTGTTTAAA
AGCCGACTGTGCTGGTATGATTACGTGGAGGTCCGGGATGGTTACTGGAGAAAAGCCCCCCTTTTGGG
CAGGTTTTGTGGCGATAAGATCCCGGAGCCCCTCGTCTCCACGGACAGCCGGCTCTGGGTGGAGTTCC
GCAGCAGCAGCAACATCTTGGGCAAGGGCTTCTTTGCAGCGTACGAAGCTACCTGCGGGGGAGACATG
AACAAAGATGCCGGTCAGATTCAATCTCCCAACTATCCGGATGACTACAGACCTTCCAAGGAATGTGT
CTGGAGGATTACGGTTTCAGAGGGGTTTCACGTGGGACTTACCTTCCAAGCTTTTGAGATTGAAAGGC
ACGACAGCTGTGCATATGACTACCTGGAAGTCCGGGATGGCCCCACGGAAGAGAGTGCCCTGATCGGC
CACTTTTGTGGCTATGAGAAGCCGGAGGATGTGAAATCGAGCTCCAACAGACTGTGGATGAAGTTTGT
GTCCGATGGCTCTATCAATAAAGCGGGCTTTGCAGCCAATTTTTTCAAGGAGGTGGATGAGTGTTCCT
GGCCAGATCACGGCGGGTGCGAGCATCGCTGTGTGAACACGCTGGGCAGCTACAAGTGTGCCTGTGAC
CCTGGCTACGAGCTGGCCGCCGATAAGAAGATGTGTGAAGTGGCCTGTGGCGGTTTCATTACCAAGCT
GAATGGAACCATCACCAGCCCTGGGTGGCCGAAGGAGTATCCCACAAACAAAAACTGTGTCTGGCAGG
TGGTGGCCCCCACTCAGTACCGGATCTCCCTTCAGTTTGAAGTGTTTGAACTGGAAGGCAATGACGTC
TGTAAGTACGACTTTGTAGAGGTGCGCAGCGGCCTGTCCCCCGACGCCAAGCTGCACGGCAGGTTCTG
CGGCTCTGAGACGCCGGAGGTCATCACCTCGCAGAGCAACAACATGCGCGTGGAGTTCAAGTCCGACA
ACACCGTCTCCAAGCGCGGCTTCAGGGCCCACTTCTTCTCAGATAAGGACGAGTGTGCCAAGGACAAC
GGCGGGTGTCAGCATGAGTGCGTCAACACCTTCGGGAGCTACCTGTGCAGGTGCAGAAACGGCTACTG
GCTCCACGAGAATGGGCATGACTGCAAAGAGGCTGGCTGTGCACACAAGATCAGCAGTGTGGAGGGGA
CCCTGGCGAGCCCCAACTGGCCTGACAAATACCCCAGCCGGAGGGAGTGTACCTGGAACATCTCTTCG
ACTGCAGGCCACAGAGTGAAACTCACCTTTAATGAGTTTGAGATCGAGCAGCACCAGGAATGTGCCTA
TGACCACCTGGAAATGTATGACGGGCCGGACAGCCTGGCCCCCATTCTGGGCCGTTTCTGCGGTAGCA
AGAAACCAGACCCCACGGTGGCTTCCGGCAGCAAGTGCGGGGGCAGGCTGAAGGCTGAAGTGCAGACC
AAAGAGCTCTATTCCCACGCCCAGTTTGGGGACAACAACTACCCGAGCGAGGCCCGCTGTGACTGGGT
GATCGTGGCAGAGGACGGCTACGGCGTGGAGCTGACATTCCGGACCTTTGAGGTTGAGGAGGAGGCCG
ACTGCGGCTACGACTACATGGAAGCCTACGACGGCTACGACAGCTCAGCGCCCAGGCTCGGCCGCTTC
TGTGGCTCTGGGCCATTAGAAGAAATCTACTCTGCAGGTGATTCCCTGATGATTCGATTCCGCACAGA
TGACACCATCAACAAGAAAGGCTTTCATGCCCGATACACCAGCACCAAGTTCCAGGATGCCCTGCACA
TGAAGAAATAGTGCTGAT

NOV8d, CG50235-03

| | | | |
|---|---|---|---|
| Protein Sequence | SEQ ID NO: 92 | 970 aa | MW at 108564.0kD |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKGSQRAIFKQAMRHWE
KHTCVTFIERTDEESFIVFSYRTCGCCSYVGRRGGGPQAISIGKNCDKFGIVAHELGHVVGFWHEHTR
PDRDQHVTIIRENIQPGQEYNFLKMEAGEVSSLGETYDFDSINHYARNTFSRGVFLDTILPRQDDNGV
RPTIGQRVRLSQGDIAQARKLYKCPGPTCAFVSQKTSICLLHFSPTCSEGFGWQRACGETLQDTTGNF
SAPGFPNGYPSYSHCVWRISVTPGEKIVLNFTSMDLFKSRLCWYDYVEVRDGYWRKAPLLGRFCGDKI
PEPLVSTDSRLWVEFRSSSNILGKGFFAAYEATCGGDMNKDAGQIQSPNYPDDYRPSKECVWRITVSE
GFHVGLTFQAFEIERHDSCAYDYLEVRDGPTEESALIGHFCGYEKPEDVKSSSNRLWMKFVSDGSINK
AGFAANFFKEVDECSWPDHGGCEHRCVNTLGSYKCACDPGYELAADKKMCEVACGGFITKLNGTITSP
GWPKEYPTNKNCVWQVVAPTQYRISLQFEVFELEGNDVCKYDFVEVRSGLSPDAKLHGRFCGSETPEV
ITSQSNNMRVEFKSDNTVSKRGFRAHFFSDKDECAKDNGGCQHECVNTFGSYLCRCRNGYWLHENGHD
CKEAGCAHKISSVEGTLASPNWPDKYPSRRECTWNISSTAGHRVKLTFNEFEIEQHOECAYDHLEMYD
GPDSLAPILGRFCGSKKPDPTVASGSKCGGRLKAEVQTKELYSHAQFGDNNYPSEARCDWVIVAEDGY
GVELTFRTFEVEEEADCGYDYMEAYDGYDSSAPRLGRFCGSGPLEEIYSAGDSLMIRFRTDDTINKKG
FHARYTSTKFQDALHMKK

| | |
|---|---|
| SEQ ID NO: 93 | 3120 bp |

| | | |
|---|---|---|
| NOV8e, SNP13377383 of | ORF Start: ATG at 256 | ORF Stop: TGA at 2719 |

| | | |
|---|---|---|
| CG50235-04, DNA Sequence | SNP Pos: 2201 | SNP Change: A to G |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCGGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGGCCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATTCCCACGCCCAGTTTGGGGACAACAACTACCCGAGCGA
GGCCCGCTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGATTCCCTGAT
GATTCGATTCCGCACAGATGACACCATCAACAAGAAAGGCTTTCATGCCCGATACACCAGCACCAAGT
TCCAGGATGCCCTGCACATGAAGAAATAGTGCTGATGTTCTTGAAAGACAGAAACTGAGA

| | | |
|---|---|---|
| NOV8e, SNP13377383 of | SEQ ID NO: 94 | MW at 91432.3kD |

| | | | |
|---|---|---|---|
| CG50235-04, Protein Sequence | SNP Pos: 649 | 821 aa | SNP Change: Gln to Arg |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAIMDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTSSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWRVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTWNISSTAGHRVKL
SAGSG

| | |
|---|---|
| SEQ ID NO: 95 | 3120 bp |

| | | |
|---|---|---|
| NOV8f, SNP13377384 of | ORF Start: ATG at 256 | ORF Stop: TGA at 2719 |

| | | |
|---|---|---|
| CG50235-04, DNA Sequence | SNP Pos: 2434 | SNP Change: G to A |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCAGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGACCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATTCCCACGCCCAGTTTGGGGACAACAACTACCCGAGCGA
GGCCCGCTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGATTCCCTGAT
GATTCGATTCCGCACAGATGACACCATCAACAAGAAAGGCTTTCATGCCCGATACACCAGCACCAGGT
TCCAGGATGCCCTGCACATGAAGAAATAGTGCTGATGTTCTTGAAAGACAGAAACTGAGA

| | | |
|---|---|---|
| NOV8f, SNP13377384 of | SEQ ID NO: 96 | MW at 91434.2kD |

| | | | |
|---|---|---|---|
| CG50235-04, Protein Sequence | SNP Pos: 727 | 821 aa | SNP Change: Ala to Thr |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTANDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDNGGCEHRCVNTLGSYKCACDPGYELAAD
KKNCEVACGGFITKLNGTITSPGWPKEYPTNKNCVWQVVAPAQYRISLQFEVFELEGNDVCKYDFVEV
RSGLSPDAKLHGRFCGSETPEVITSQSNMRVEFKSDNTVSKRGFRTHFFSDKDECAKDDNGGCQHECV
NTFGSYLCRCRNGYWLHENGHDCKEAGCAHKISSVEGTLASPNWPDKYPSRRECTWNISSTAGHRVKL
SAGSG

| | |
|---|---|
| SEQ ID NO: 97 | 3120 bp |

| | | |
|---|---|---|
| NOV8g, SNP13377385 of | ORF Start: ATG at 256 | ORF Stop: TGA at 2719 |

| | | |
|---|---|---|
| CG50235-04, DNA Sequence | SNP Pos: 2751 | SNP Change: T to C |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCAGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGGCCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATCCCCACGCCCAGTTTGGGGACAACAACTACCCGAGCGA
GGCCCGCTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGATTCCCTGAT
GATTCGATTCCGCACAGATGACACCATCAACAAGAAAGGCTTTCATGCCCGATACACCAGCACCAAGT
TCCAGGATGCCCTGCACATGAAGAAATAGTGCTGATGTTCTTGAAAGACAGAAACTGAGA

| | | |
|---|---|---|
| NOV8g, SNP13377385 of | +TL,19 | MW at 91404.2kD |

| | | | |
|---|---|---|---|
| CG50235-04, Protein Sequence | SEQ ID NO: 98 | 821 aa | SNP Change: no change |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDNNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTWNISSTAGHRVKL
SAGSG

| | |
|---|---|
| SEQ ID NO: 99 | 3120 bp |

| | | |
|---|---|---|
| NOV8h, SNP13377386 of | ORF Start: ATG at 256 | ORF Stop: TGA at 2719 |

| | | |
|---|---|---|
| CG50235-04, DNA Sequence | SNP Pos: 2794 | SNP Change: G to A |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCAGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGGCCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATTCCCACGCCCAGTTTGGGGACAACAACTACCCGAGCGA
GGCCCACTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGATTCCCTGAT
GATTCGATTCCGCACAGATGACACCATCAACAAGAAAGGCTTTCATGCCCGATACACCAGCACCAAGT
TCCAGGATGCCCTGCACATGAAGAAATAGTGCTGATGTTCTTGAAAGACAGAAACTGAGA

| | |
|---|---|
| NOV8h, SNP13377386 of | MW at 91404.2kD |

| | | | |
|---|---|---|---|
| CG50235-04, Protein Sequence | SEQ ID NO: 100 | 821 aa | SNP Change: no change |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGRENTTLLHSPGTL
HAAAKTFSPRVRRATTSRTERIWPGGVIPYVIGGNFTGSQRAIFKQAMRHWEKHTCVTFIERTDEESF
IVFSYRTCGCCSYVGRRGGGPQAISIGKNCDKFGIVAHELGHVVGFWHEHTRPDRDQHVTIIRENIOP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTNNISSTAGHRVKL
SAGSG

| | |
|---|---|
| SEQ ID NO: 101 | 3120 bp |

| | | |
|---|---|---|
| NOV8i, SNP13377387 of | ORF Start: ATG at 256 | ORF Stop: TGA at 2719 |

| | | |
|---|---|---|
| CG50235-04, DNA Sequence | SNP Pos: 2983 | SNP Change: A to G |

TAACCTGTCTCACGACGGTCTAAACCCAGGCAGCCTCGGCCGCCGGGCAAGTAGCTCCGAGCGGCTGC
TTCCCGGTTGCCTCGAAGAAGACAGGGGGCGCCGCGCTCCGCTTGCTCCGCGCCTGAGCCATGCCCAG
CAGCCCTGTGTAACCACCGAGTCCCGGCCGGAGCCGACCGACCCAGTGTGCGCCGTCTTTCGGCCGAG
CTGAGCTTTCGTGCACGCAACTCCCTCTGCCCCAGCCGGCCCCGCGCCACCATGCCCCGGGCGACTGC
ACTTGGGGCCCTGGTGTCACTGCTGCTGCTGCTGCCGCTGCCTCGCGGCGCCGGGGGACTCGGGGAGC
GCCCGGACGCCACCGCAGACTACTCAGAGCTGGACGGCGAGGAGGGCACGGAGCAGCAGCTGGAGCAT
TACCACGACCCTTGCAAAGCCGCTGTCTTTTGGGGAGACATTGCCTTAGATGAAGATGACTTGAAGCT
GTTTCACATTGACAAAGCCAGAGACTGGACCAAGCAGACAGTGGGGGCAACAGGACACAGCACAGGTG
GGCTTGAAGAGCAGGCATCTGAGAGCAGCCCAGACACCACAGCCATGGACACTGGCACCAAGGAAGCT
GGAAAGGATGGCCGGGAGAATACCACACTCCTGCACAGCCCTGGGACCTTGCATGCCGCAGCCAAGAC
CTTCTCTCCCCGGGTCCGAAGAGCCACAACCTCAAGGACAGAGAGGATATGGCCTGGAGGAGTCATCC
CCTACGTCATTGGAGGGAACTTCACTGGGAGCCAGAGGGCCATTTTTAAGCAGGCCATGAGACACTGG
GAGAAGCACACCTGTGTGACCTTCATAGAAAGGACGGATGAGGAAAGCTTTATTGTATTCAGTTACAG
AACCTGTGGCTGTTGCTCCTATGTTGGGCGCCGAGGAGGAGGCCCACAGGCCATATCCATTGGGAAGA
ACTGTGACAAGTTTGGCATTGTGGCTCACGAGCTGGGCCATGTGGTTGGGTTTTGGCATGAACACACC
CGGCCAGACAGAGACCAACATGTCACCATCATCAGGGAAAACATCCAGCCAGGTCAGGAGTATAATTT
CTTAAAAATGGAAGCTGGGGAAGTGAGCTCTCTGGGAGAGACATACGACTTTGACAGCATCATGCACT
ACGCCCGGAACACCTTCTCAAGAGGAGTTTTCTTAGACACCATCCTTCCCCGTCAAGATGACAATGGC
GTCAGGCCAACCATTGGCCAGCGCGTGCGGCTCAGTCAGGGAGACATAGCTCAAGCCCGGAAGCTGTA
CAAATGCCCAGCGTGTGGGGAGACCCTGCAGGACACAACGGGAAACTTTTCTGCACCTGGTTTCCCAA
ATGGGTACCCATCTTACTCCCACTGCGTCTGGAGGATCTCGGTCACCCCAGGGGAAAAGATCGTATTA
AACTTCACATCCATGGATTTGTTTAAAAGCCGACTGTGCTGGTATGATTACGTGGAGGTCCGGGATGG
TTACTGGAGAAAAGCCCCCCTTTTGGGCAGGTTTTGTGGCGATAAGATCCCGGAGCCCCTCGTCTCCA
CGGACAGCCGGCTCTGGGTGGAGTTCCGCAGCAGCAGCAACATCTTGGGCAAGGGCTTCTTTGCAGCG
TACGAAGCTACCTGCGGGGGAGACATGAACAAAGATGCCGGTCAGATTCAATCTCCCAACTATCCGGA
TGACTACAGACCTTCCAAGGAATGTGTCTGGAGGATTACGGTTTCGGAGGGGTTTCACGTGGGACTTA
CCTTCCAAGCTTTTGAGATTGAAAGGCACGACAGCTGTGCATATGACTACCTGGAAGTCCGGGATGGC
CCCACGGAAGAGAGTGCCCTGATCGGCCACTTTTGTGGCTATGAGAAGCCGGAGGATGTGAAATCGAG
CTCCAACAGACTGTGGATGAAGTTTGTGTCCGATGGCTCTATCAATAAAGCGGGCTTTGCAGCCAATT
TTTTCAAGGAGGTGGATGAGTGTTCCTGGCCAGATCACGGCGGGTGCGAACATCGCTGTGTGAACACG
CTGGGCAGCTACAAGTGTGCCTGTGACCCTGGCTACGAGCTGGCCGCCGATAAGAAGATGTGTGAAGT
GGCCTGTGGCGGTTTCATTACCAAGCTGAATGGAACCATCACCAGCCCTGGGTGGCCGAAGGAGTATC
CCACAAACAAAAACTGTGTCTGGCAGGTGGTGGCCCCCGCTCAGTACCGGATCTCCCTTCAGTTTGAA
GTGTTTGAACTGGAAGGCAATGACGTCTGTAAGTACGACTTTGTAGAGGTGCGCAGCGGCCTGTCCCC
CGACGCCAAGCTGCACGGCAGGTTCTGCGGCTCTGAGACGCCGGAAGTCATCACCTCGCAGAGCAACA
ACATGCGCGTGGAGTTCAAGTCCGACAACACCGTCTCCAAGCGCGGCTTCAGGGCCCACTTCTTCTCA
GATAAGGACGAGTGTGCCAAGGACAACGGCGGGTGTCAGCATGAGTGCGTCAACACCTTCGGGAGCTA
CCTGTGCAGGTGCAGAAACGGCTACTGGCTCCACGAGAATGGGCATGACTGCAAAGAGGCTGGCTGTG
CACACAAGATCAGCAGTGTGGAGGGGACCCTGGCGAGCCCCAACTGGCCTGACAAATACCCCAGCCGG
AGGGAGTGTACCTGGAACATCTCTTCGACTGCAGGCCACAGAGTAAAACTCAGTGCGGGGTCAGGCTG
AAGGCTGAAGTGCAGACCAAAGAGCTCTATTCCCACGCCCAGTTTGGGGACAACAACTACCCGAGCGA
GGCCCGCTGTGACTGGGTGATCGTGGCAGAGGACGGCTACGGCGTGGAGCTGACATTCCGGACCTTTG
AGGTTGAGGAGGAGGCCGACTGCGGCTACGACTACATGGAAGCCTACGACGGCTACGACAGCTCAGCG
CCCAGGCTCGGCCGCTTCTGTGGCTCTGGGCCATTAGAAGAAATCTACTCTGCAGGTGGTTCCCTGAT
GATTCGATTCCGCACAGATGACACCATCAACAAGAAAGGCTTTCATGCCCGATACACCAGCACCAAGT
TCCAGGATGCCCTGCACATGAAGAAATAGTGCTGATGTTCTTGAAAGACAGAAACTGAGA

| | |
|---|---|
| NOV8i, SNP13377387 of | MW at 91404.2kD |

| | | | |
|---|---|---|---|
| CG50235-04, Protein Sequence | SEQ ID NO: 102 | 821 aa | SNP Change: no change |

MPRATALGALVSLLLLLPLPRGAGGLGERPDATADYSELDGEEGTEQQLEHYHDPCKAAVFWGDIALD
EDDLKLFHIDKARDWTKQTVGATGHSTGGLEEQASESSPDTTAMDTGTKEAGKDGREMTTLLHSPGTL
HAAAKTFSPRVRRATTSRTERIWPGGVIPYVIGGNFTGSQRAIFKQANRHWEKHTCVTFIERTDEESF
IVFSYRTCGCCSYVGRRGGGPQAISIGKNCDKFGIVAHELGHVVGFWHEHTRPDRDQHVTIIRENIQP
GQEYNFLKMEAGEVSSLGETYDFDSIMHYARNTFSRGVFLDTILPRQDDNGVRPTIGQRVRLSQGDIA
QARKLYKCPACGETLQDTTGNFSAPGFPNGYPSYSHCVWRISVTPGEKIVLNFTSMDLFKSRLCWYDY
VEVRDGYWRKAPLLGRFCGDKIPEPLVSTDSRLWVEFRSSSNILGKGFFAAYEATCGGDMNKDAGQIQ
SPNYPDDYRPSKECVWRITVSEGFHVGLTFQAFEIERHDSCAYDYLEVRDGPTEESALIGHFCGYEKP
EDVKSSSNRLWMKFVSDGSINKAGFAANFFKEVDECSWPDHGGCEHRCVNTLGSYKCACDPGYELAAD
KKMCEVACGGFITKLNGTITSPGWPKEYPTNKNCVWQVVAPAQYRISLQFEVFELEGNDVCKYDFVEV
RSGLSPDAKLHGRFCGSETPEVITSQSNNMRVEFKSDNTVSKRGFRAHFFSDKDECAKDNGGCQHECV
NTFGSYLCRCRNGYWLHENGHDCKEAGCAHKISSVEGTLASPNWPDKYPSRRECTWNISSTAGHRVKL
SAGSG

A ClustalW comparison of the above protein sequences yields the following sequence alignment shown in Table 8B.

TABLE 8B
Comparison of the NOV8 protein sequences.

| | |
|---|---|
| NOV8a | MPRATALGALVSLLLLLPLPRGAGGLGERPDATADYSELDGEEGTEQQLEHYHDPCKAAV |
| NOV8b | MPRATALGALVSLLLLLPLPRGAGGLGERPDATADYSELDGEEGTEQQLEHYHDPCKAAV |
| NOV8c | MPRATALGALVSLLLLLPLPRGAGGLGERPDATADYSELDGEEGTEQQLEHYHDPCKAAV |
| NOV8d | MPRATALGALVSLLLLLPLPRGAGGLGERPDATADYSELDGEEGTEQQLEHYHDPCKAAV |
| NOV8a | FWGDIALDEDDLKLFHIDKARDWTKQTVGATGHSTGGLEEQASESSPDTTAMDTGTKEAG |
| NOV8b | FWGDIALDEDDLKLFHIDKARDWTKQTVGATGHSTGGLEEQASESSPDTTAMDTGTKEAG |
| NOV8C | FWGDIALDEDDLKLFHIDKARDWTKQTVGATGHSTGGLEEQASESSPDTTAMDTGTKEAG |
| NOV8d | FWGDIALDEDDLKLFHIDKARDWTKQTVGATGHSTGGLEEQASESSPDTTAMDTGTKEAG |
| NOV8a | KDGRENTTLLHSPGTLHAAAKTFSPRVRRATTSRTERIWPGGVIPYVIGGNFTGSQRAIF |
| NOV8b | KDGRENTTLLHSPGTLHAAAKTFSPRVRRATTSRTERIWPGGVIPYVIGGNFTGSQRAIF |
| NOV8c | KDGRENTTLLHSPGTLHAAAKTFSPRVRRATTSRTERIWPGGVIPYVIGGNFTGSQRAIF |
| NOV8d | K----------------------------------------------------GSQRAIF |
| NOV8a | KQAMRHWEKHTCVTFIERTDEESFIVFSYRTCGCCSYVGRRGGGPQAISIGKNCDKFGIV |
| NOV8b | KQAMRHWEKHTCVTFIERTDEESFIVFSYRTCGCCSYVGRRGGGPQAISIGKNCDKFGIV |
| NOV8c | KQAMRHWEKHTCVTFIERTDEESFIVFSYRTCGCCSYVGRRGGGPQAISIGKNCDKFGIV |
| NOV8d | KQAMRHWEKHTCVTFIERTDEESFIVFSYRTCGCCSYVGRRGGGPQAISIGKNCDKFGIV |
| NOV8a | AHELGHVVGFWHEHTRPDRDQHVTIIRENIQPGQEYNFLKMEAGEVSSLGETYDFDSIMH |
| NOV8b | AHELGHVVGFWHEHTRPDRDQHVTIIRENIQPGQEYNFLKMEAGEVSSLGETYDFDSIMH |
| NOV8c | AHELGHVVGFWHEHTRPDRDQHVTIIRENIQPGQEYNFLKMEAGEVSSLGETYDFDSIMH |
| NOV8d | AHELGHVVGFWHEHTRPDRDQHVTIIRENIQPGQEYNFLKMEAGEVSSLGETYDFDSIMH |
| NOV8a | YARNTFSRGVFLDTILPRQDDNGVRPTIGQRVRLSQGDIAQARKLYKCP----------- |
| NOV8b | YARNTFSRGVFLDTILPRQDDNGVRPTIGQRVRLSQGDIAQARKLYKCP----------- |
| NOV8c | YARNTFSRGVFLDTILPRQDDNGVRPTIGQRVRLSQGDIAQARKLYKCP----------- |
| NOV8d | YARNTFSRGVFLDTILPRQDDNGVRPTIGQRVRLSQGDIAQARKLYKCPGPTCAFVSQKT |
| NOV8a | -------------------ACGETLQDTTGNFSAPGFPNGYPSYSHCVWRISVTPGEKIV |
| NOV8b | -------------------ACGETLQDTTGNFSAPGFPNGYPSYSHCVWRISVTPGEKIV |
| NOV8c | -------------------ACGETLQDTTGNFSAPGFPNGYPSYSHCVWRISVTPGEKIV |
| NOV8d | SICLLHFSPTCSEGFGWQRACGETLQDTTGNFSAPGFPNGYPSYSHCVWRISVTPGEKIV |
| NQV8a | LNFTSMDLFKSRLCWYDYVEVRDGYWRKAPLLGRFCGDKIPEPLVSTDSRLWVEFRSSSN |
| NOV8b | LNFTSMDLFKSRLCWYDYVEVRDGYWRKAPLLGRFCGDKIPEPLVSTDSRLWVEFRSSSN |
| NOV8c | LNFTSMDLFKSRLCWYDYVEVRDGYWRKAPLLGRFCGDKIPEPLVSTDSRLWVEFRSSSN |
| NOV8d | LNFTSMDLFKSRLCWYDYVEVRDGYWRKAPLLGRFCGDKIPEPLVSTDSRLWVEFRSSSN |
| NOV8a | ILGKGFFAAYEATCGGDMNKDAGQIQSPNYPDDYRPSKECVWRITVSEGFHVGLTFQAFE |
| NOV8b | ILGKGFFAAYEATCGGDMNKDAGQIQSPNYPDDYRPSKECVWRITVSEGFHVGLTFQAFE |
| NOV8c | ILGKGFFAAYEATCGGDMNKDAGQIQSPNYPDDYRPSKECVWRITVSEGFHVGLTFQAFE |
| NOV8d | ILGKGFFAAYEATCGGDMNKDAGQIQSPNYPDDYRPSKECVWRITVSEGFHVGLTFQAFE |
| NOV8a | IERHDSCAYDYLEVRDGPTEESALIGHFCGYEKPEDVKSSSNRLWMKFVSDGSINKAGFA |
| NOV8b | IERHDSCAYDYLEVRDGPTEESALIGHFCGYEKPEDVKSSSNRLWMKFVSDGSINKAGFA |
| NOV8c | IERHDSCAYDYLEVRDGPTEESALIGHFCGYEKPEDVKSSSNRLWMKFVSDGSINKAGFA |
| NOV8d | IERHDSCAYDYLEVRDGPTEESALIGHFCGYEKPEDVKSSSNRLWMKFVSDGSINKAGFA |
| NOV8a | ANFFKEVDECSWPDHGGCENRCVNTLGSYKCACDPGYELAADKKMCEVACGGFITKLNGT |
| NOV8b | ANFFKEVDECSWPDHGGCEHRCVNTLGSYKCACDPGYELAADKKMCEVACGGFITKLNGT |
| NOV8c | ANFFKEVDECSWPDHGGCEHRCVNTLGSYKCACDPGYELAADKKMCEVACGGFITKLNGT |
| NOV8d | ANFFKEVDECSWPDHGGCEHRCVNTLGSYKCACDPGYELAADKKMCEVACGGFITKLNGT |
| NOV8a | ITSPGWPKEYPTMKNCVWQVVAPAQYRISLQFEVFELEGNDVCKYDFVEVRSGLSPDAKL |
| NOV8b | ITSPGWPKEYPTNKNCVWQVVAPTQYRISLQFEVFELEGNDVCKYDFVEVRSGLSPDAKL |
| NOV8c | ITSPGWPKEYPTNKNCVWQVVAPAQYRISLQFEVFELEGNDVCKYDFVEVRSGLSPDAKL |
| NOV8d | ITSPGWPKEYPTNKNCVWQVVAPTQYRISLQFEVFELEGNDVCKYDFVEVRSGLSPDAKL |
| NOV8a | HGRFCGSETPEVITSQSNNMRVEFKSDNTVSKRGFRAHFFSDKDECAKDNGGCQHECVNT |
| NOV8b | HGRFCGSETPEVITSQSNNMRVEFKSDNTVSKRGFRAHFFSDKDECAKDNGGCQHECVNT |
| NOV8c | HGRFCGSETPEVITSQSNNMRVEFKSDNTVSKRGFRAHFFSDKDECAKDNGGCQHECVNT |
| NOV8d | HGRFCGSETPEVITSQSNNMRVEFKSDNTVSKRGFRAHFFSDKDECAKDNGGCQHECVNT |
| NOV8a | FGSYLCRCRNGYWLHENGHDCKEAGCAHKISSVEGTLASPNWPDKYPSRRECTWNISSTA |
| NOV8b | FGSYLCRCRNGYWLHENGHDCKEAGCAHKISSVEGTLASPNWPDKYPSRRECTWNISSTA |
| NOV8c | FGSYLCRCRNGYWLHENGHDCKEAGCAHKISSVEGTLASPNWPDKYPSRRECTWNISSTA |
| Novad | FGSYLCRCRNGYWLHENGHDCKEAGCAHKISSVEGTLASPNWPDKYPSRRECTWNISSTA |
| NOV8a | GHRVKLSAGSG------------------------------------------------- |
| NOV8b | GHRVKLTFNEFEIEQHQECAYDHLEMYDGPDSLAPILGRFCGSKKPDPTVASGSKCGGRL |
| NOV8c | GHRVKLTFNEFEIEQHQECAYDHLEMYDGPDSLAPILGRFCGSKKPDPTVASGSSMFLRF |
| NOV8d | GHRVKLTFNEFEIEQHQECAYDHLEMYDGPDSLAPILGRFCGSKKPDPTVASGSKCGGRL |
| NOV8a | ------------------------------------------------------------ |
| NOV8b | KAEVQTKELYSHAQFGDNNYPSEARCDWVIVAEDGYGVELTFRTFEVEEEADCGYDYMEA |
| NOV8c | YSDASVQRKGFQAVIISTECGGRLKAEVQTKELYSHAQFGDNNYPSEARCDWVIVAEDGYG |
| NOV8d | KAEVQTKELYSHAQFGDNNYPSEARCDWVIVAEDGYGVELTFRTFEVEEEADCGYDYMEA |
| NOV8a | ------------------------------------------------------------ |
| NOV8b | YDGYDSSAPRLGRFCGSGPLEEIYSAGDSLMIRFRTDDTINKKGFHARYTSTKFQDGLHM |
| NOV8c | VELTFRTFEVEEEADCGYDYMEAYDGYDSSAPRLGRFCGSGPLEEIYSAGDSLMIRFRTD |
| NOV8d | YDGYDSSAPRLGRFCGSGPLEEIYSAGDSLMIRFRTDDTINKKGFHARYTSTKFQDALHM |
| NOV8a | ------------------------- |
| NOV8b | KK----------------------- |
| NOV8c | DTINKKGFHARYTSTKFQDALHMKK |
| NOV8d | KK----------------------- |
| NOV8a | (SEQ ID NO: 86) |
| NOV8b | (SEQ ID NO: 88) |
| NOV8c | (SEQ ID NO: 90) |
| NOV8d | (SEQ ID NO: 92) |

Further analysis of the NOV8a protein yielded the following properties shown in Table 8C.

TABLE 8C
Protein Sequence Properties NOV8a

```
Signa1P analysis: Cleavage site between residues 26 and 27
PSORT II analysis:
PSG: a new signal peptide prediction method
    N-region: length 3; pos. chg 1; neg. chg 0
    H-region: length 17; peak value 10.45
    PSG score: 6.05
GvH: von Heijne's method for signal seq. recognition
    GvH score (threshold: −2.1): 4.44
    possible cleavage site: between 25 and 26
>>> Seems to have a cleavable signal peptide (1 to 25)
ALOM: Klein et al's method for TM region allocation
    Init position for calculation: 26
    Tentative number of TMS(s) for the threshold 0.5: 0
    number of TMS(s) .. fixed
    PERIPHERAL Likelihood = 6.31 (at 203)
    ALOM score: 6.31 (number of TMSs: 0)
MTOP: Prediction of membrane topology (Hartmann et al.)
    Center position for calculation: 12
    Charge difference: −3.0 C(−1.0) − N( 2.0)
    N >= C: N-terminal side will be inside
MITDISC: discrimination of mitochondrial targeting seq
    R content: 2 Hyd Moment(75): 7.80
    Hyd Moment(95): 7.61G content: 5
    DIE content: 1 S/T content: 2
    Score: −4.54
Gavel: prediction of cleavage sites for mitochondrial preseq
    R-2 motif at 31 PRG|AG
NUCDISC: discrimination of nuclear localization signals
    pat4: none
    pat7: PRVRRAT (5) at 145
    bipartite: none
    content of basic residues: 11.2%
    NLS Score: −0.04
KDEL: ER retention motif in the C-terminus: none
ER Membrane Retention Signals:
    XXRR-like motif in the N-terminus: PRAT
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 76.7
COIL: Lupas's algorithm to detect coiled-coil regions
    total: 0 residues
--------------------------
Final Results (k = 9/23)
    33.3%: extracellular, including cell wall
    22.2%: Golgi
    22.2%: vacuolar
    11.1%: mitochondrial
    11.1%: endoplasmic reticulum
>> predictionfor CG50235-04 is exc (k=9)
```

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| AAY32240 | Human tolloid-like protein | 1 . . . 817 | 816/817 (99%) | 0.0 |
| | mT11-2 - Homo sapiens, 1015 | 1 . . . 817 | 817/817 (99%) | |
| | aa. [WO9951730-A2, 14-OCT- | | | |
| | 1999] | | | |
| ABG79187 | Human tolloid-like 2-like protein | 1 . . . 817 | 815/817 (99%) | 0.0 |
| | #1 - Homo sapiens, 992 aa. | 1 . . . 817 | 816/817 (99%) | |
| | [WO200264791-A2, 22-AUG- | | | |
| | 2002] | | | |
| ABG79188 | Human tolloid-like 2-like protein | 1 . . . 817 | 763/847 (90%) | 0.0 |
| | #2 - Homo sapiens, 970 aa. | 1 . . . 795 | 764/847 (90%) | |
| | [WO200264791 -A2, 22-AUG- | | | |
| | 2002] | | | |
| AAY32241 | Mouse tolloid-like protein mT11- | 1 . . . 817 | 728/817 (89%) | 0.0 |
| | 2-Mus musculus, 1012 aa. | 1 . . . 814 | 759/817(92%) | |
| | [WO9951730-A2, 14-OCT- | | | |
| | 1999] | | | |
| AAW40224 | Human tolloid-like (T11) protein - | 35 . . . 817 | 584/783 (74%) | 0.0 |
| | Homo sapiens, 1013 aa. | 34 . . . 815 | 666/783 (84%) | |
| | [WO9745528-A2, 04-DEC- | | | |
| | 1997] | | | |

In a BLAST search of public sequence databases, the NOV8a protein was found to have homology to the proteins shown in the BLASTP data in Table 8E.

TABLE 8E
Public BLASTP Results for NOV8a

| | | | Identities/ | |
|---|---|---|---|---|
| | | NOV8a | Similarities | |
| Protein | | Residues/ | for the | |
| Accession | Protein/ | Match | Matched | Expect |
| Number | Organism/Length | Residues | Portion | Value |
| Q9Y6L7 | Tolloid-like 2 protein - | 1 . . . 817 | 816/817 | 0.0 |
| | Homo sapiens | 1 . . . 817 | (99%) | |
| | (Human), 1015 aa. | | 817/817 | |
| | | | | (99%) |
| Q9WVM6 | Tolloid-like-2 protein - | 1 . . . 817 | 728/817 | 0.0 |
| | Mus musculus | 1 . . . 814 | (89%) | |
| | (Mouse), 1012 aa. | | 759/817 | |
| | | | | (92%) |
| Q9UQ00 | Hypothetical protein | 90 . . . 817 | 726/728 | 0.0 |
| | KIAA0932 - Homo | 1 . . . 728 | (99%) | |
| | sapiens (Human), 926 | | 727/728 | |
| | aa (fragment). | | (99%) | |
| O57382 | Xolloid - Xenopus | 52 . . . 816 | 596/768 | 0.0 |
| | laevis (African clawed | 52 . . . 819 | (77%) | |
| | frog), 1019 aa. | | 680/768 | |
| | | | | (87%) |
| Q8J128 | Xolloid-like | 35 . . . 817 | 597/783 | 0.0 |
| | metalloprotease - | 29 . . . 810 | (76%) | |
| | Xenopus laevis | | 679/783 | |
| | Africian clawed | | (86%) | |
| | frog), 1007 aa. | | | |

PFam analysis indicates that the NOV8a protein contains the domains shown in the Table 8F.

TABLE 8F
Domain Analysis of NOV8a

| | | Identities/ | |
|---|---|---|---|
| | NOV8a | Similarities for | |
| Pfam Domain | Match Region | the Matched Region | Expect Value |
| Astacin | 157 . . . 350 | 100/202 (50%) | 9.5e-114 |
| | | 172/202 (85%) | |
| CUB | 351 . . . 460 | 50/116(43%) | 2.4e-52 |
| | | 99/116 (85%) | |
| CUB | 464 . . . 573 | 62/116 (53%) | 3.1e-60 |
| | | 98/116 (84%) | |
| EGF | 580 . . . 616 | 17/47 (36%) | 1.2e-05 |
| | | 29/47 (62%) | |
| CUB | 620 . . . 729 | 57/116 (49%) | 4.7e-58 |
| | | 101/116 (87%) | |
| EGF | 736 . . . 771 | 18/47 (38%) | 7.4e-07 |
| | | 31/47 (66%) | |

### Example 9

The NOV9 clone was analyzed, and the nucleotide and encoded polypeptide sequences are shown in Table 9A.

TABLE 9A
NOV9 Sequence Analysis

| | | |
|---|---|---|
| NOV9a, CG50249-01 | SEQ ID NO: 103 | 1953 bp |

| | | |
|---|---|---|
| DNA Sequence | ORF Start: ATG at 16 | ORF Stop: TAA at 1930 |

GTCTGAGTCACAGAGATGGGCAAGATCGAGAACAACGAGAGGGTGATCCTCAATGTCGGGGGCACCCG
GCACGAAACCTACCGCAGCACCCTCAAGACCCTGCCTGGAACACGCCTGGCCCTTCTTGCCTCCTCCG
AGCCCCCAGGCGACTGCTTGACCACGGCGGGCGACAAGCTGCAGCCGTCGCCGCCTCCACTGTCGCCG
CCGCCGAGAGCGCCCCCGCTGTCCCCCGGGCCAGGCGGCTGCTTCGAGGGCGGCGCGGGCAACTGCAG
TTCCCGCGGCGGCAGGGCCAGCGACCATCCCGGTGGCGGCCGCGAGTTCTTCTTCGACCGGCACCCGG
GCGTCTTCGCCTATGTGCTCAATTACTACCGCACCGGCAAGCTGCACTGCCCCGCAGACGTGTGCGGG
CCGCTCTTCGAGGAGGAGCTGGCCTTCTGGGGCATCGACGAGACCGACGTGGAGCCCTGCTGCTGGAT
GACCTACCGGCAGCACCGCGACGCCGAGGAGGCGCTGGACATCTTCGAGACCCCCGACCTCATTGGCG
GCGACCCCGGCGACGACGAGGACCTGGCGGCCAAGAGGCTGGGCATCGAGGACGCGGCGGGGCTCGGG
GGCCCGGACGGCAAATCTGGCCGCTGGAGGAGGCTGCAGCCCCGCATGTGGGCCCTCTTCGAAGACCC
CTACTCGTCCAGAGCCGCCAGGTTTATTGCTTTTGCTTCTTTATTCTTCATCCTGGTTTCAATTACAA
CTTTTTGCCTGGAAACACATGAAGCTTTCAATATTGTTAAAAACAAGACAGAACCAGTCATCAATGGC
ACAAGTGTTGTTCTACAGTATGAAATTGAAACGGATCCTGCCTTGACGTATGTAGAAGGAGTGTGTGT
GGTGTGGTTTACTTTTGAATTTTTAGTCCGTATTGTTTTTTCACCCAATAAACTTGAATTCATCAAAA
ATCTCTTGAATATCATTGACTTTGTGGCCATCCTACCTTTCTACTTAGAGGTGGGACTCAGTGGGCTG
TCATCCAAAGCTGCTAAAGATGTGCTTGGCTTCCTCAGGGTGGTAAGGTTTGTGAGGATCCTGAGAAT
TTTCAAGCTCACCCGCCATTTTGTAGGTCTGAGGGTGCTTGGACATACTCTTCGAGCTAGTACTAATG
AATTTTTGCTGCTGATAATTTTCCTGGCTCTAGGAGTTTTGATATTTGCTACCATGATCTACTATGCC
GAGAGAGTGGGAGCTCAACCTAACGACCCTTCAGCTAGTGAGCACACACAGTTCAAAAACATTCCCAT
TGGGTTCTGGTGGGCTGTAGTGACCATGACTACCCTGGGTTATGGGGATATGTACCCCCAAACATGGT
CAGGCATGCTGGTGGGAGCCCTGTGTGCTCTGGCTGGAGTGCTGACAATAGCCATGCCAGTGCCTGTC
ATTGTCAATAATTTTGGAATGTACTACTCCTTGGCAATGGCAAAGCAGAAACTTCCAAGGAAAAGAAA
GAAGCACATCCCTCCTGCTCCTCAGGCAAGCTCACCTACTTTTTGCAAGACAGAATTAAATATGGCCT
GCAATAGTACACAGAGTGACACATGTCTGGGCAAAGACAATCGACTTCTGGAACATAACAGATCAGTG
TTATCAGGTGACGACAGTACAGGAAGTGAGCCGCCACTATCACCCCCAGAAAGGCTCCCCATCAGACG
CTCTAGTACCAGAGACAAAAACAGAAGAGGGGAAACATGTTTCCTACTGACGACAGGTGATTACACGT
GTGCTTCTGATGGAGGGATCAGGAAAGGTTATGAAAAATCCCGAAGCTTAAACAACATAGCGGGCTTG
GCAGGCAATGCTCTGAGGCTCTCTCCAGTAACATCACCCTACAACTCTCCTTGTCCTCTGAGGCGCTC
TCGATCTCCCATCCCATCTATCTTGTAAACCAAACAACCAAACTGCATC

NOV9a, CG50249-01

| | | | |
|---|---|---|---|
| Protein Sequence | SEQ ID NO: 104 | 638 aa | MW at 70224.7kD |

MGKIENNERVILNVGGTRHETYRSTLKTLPGTRLALLASSEPPGDCLTTAGDKLQPSPPPLSPPPRAP
PLSPGPGGCFEGGAGNCSSRGGRASDHPGGGREFFFDRHPGVFAYVLNYYRTGKLHCPADVCGPLFEE
ELAFWGIDETDVEPCCWMTYRQHRDAEEALDIFETPDLIGGDPGDDEDLAAKRLGIEDAAGLGGPDGK
SGRWRRLQPRMWALFEDPYSSRAARFIAFASLFFILVSITTFCLETHEAFNIVKNKTEPVINGTSVVL
QYEIETDPALTYVEGVCVVWFTFEFLVRIVFSPNKLEFIKNLLNIIDFVAILPFYLEVGLSGLSSKAA
KDVLGFLRVVRFVRILRIFKLTRHFVGLRVLGHTLRASTNEFLLLIIFLALGVLIFATMIYYAERVGA
QPNDPSASEHTQFKNIPIGFWWAVVTMTTLGYGDMYPQTWSGMLVGALCALAGVLTIAMPVPVIVNNF
GMYYSLAMAKQKLPRKRKKHIPPAPQASSPTFCKTELNMACNSTQSDTCLGKDNRLLEHNRSVLSGDD
STGSEPPLSPPERLPIRRSSTRDKNRRGETCFLLTTGDYTCASDGGIRKGYEKSRSLNNIAGLAGNAL
RLSPVTSPYNSPCPLRRSRSPIPSIL

| | | |
|---|---|---|
| NOV9b, 207885588 | SEQ ID NO: 105 | 1815 bp |

| | | |
|---|---|---|
| DNA Sequence | ORF Start: at 1 | ORF Stop: end of sequence |

AGATCTCCCACCATGGGCAAGATCGAGAACAACGAGAGGGTGATCCTCAATGTCGGGGGCACCCGGCA
CGAAACCTACCGCAGCACCCTCAAGACCCTGCCTGGAACACGCCTGGCCCTTCTTGCCTCCTCCGAGC
CCCCAGGCAACTGCAGTTCCCGCGGCGGCAGGGCCAGCGACCATCCCGGTGGCGGCCGCGAGTTCTTC
TTCGACCGGCACCCGGGCGTCTTCGCCTATGTGCTCAATTACTACCGCACCGGCAAGCTGCACTGCCC
CGCAGACGTGTGCGGGCCGCTCTTCGAGGAGGAGCTGGCCTTCTGGGGCATCGACGAGACCGACGTGG
AGCCCTGCTGCTGGATGACCTACCGGCAGCACCGCGACGCCGAGGAGGCGCTGGACATCTTCGAGACC
CCCGACCTCATTGGCGGCGACCCCGGCGACGACGAGGACCTGGCGGCCAAGAGGCTGGGCATCGAGGA
CGCGGCGGGGCTCGGGGGCCCCGACGGCAAATCTGGCCGCTGGAGGAGGCTGCAGCCCCGCATGTGGG
CCCTCTTCGAAGACCCCTACTCGTCCAGAGCCGCCAGGTTTATTGCTTTTGCTTCTTTATTCTTCATC
CTGGTTTCAATTACAACTTTTTGCCTGGAAACACATGAAGCTTTCAATATTGTTAAAAACAAGACAGA
ACCAGTCATCAATGGCACAAGTGTTGTTCTACAGTATGAAATTGAAACGGATCCTGCCTTGACGTATG
TAGAAGGAGTGTGTGTGGTGTGGTTTACTTTTGAATTTTTAGTCCGTATTGTTTTTTCACCCAATAAA
CTTGAATTCATCAAAAATCTCTTGAATATCATTGACTTTGTGGCCATCCTACCTTTCTACTTAGAGGT
GGGACTCAGTGGGCTGTCATCCAAAGCTGCTAAAGATGTGCTTGGCTTCCTCAGGGTGGTAAGGTTTG
TGAGGATCCTGAGAATTTTCAAGCTCACCCGCCATTTTGTAGGTCTGAGGGTGCTTGGACATACTCTT
CGAGCTAGTACTAATGAATTTTTGCTGCTGATAATTTTCCTGGCTCTAGGAGTTTTGATATTTGCTAC
CATGATCTACTATGCCGAGAGAGTGGGAGCTCAACCTAACGACCCTTCAGCTAGTGAGCACACACAGT
TCAAAAACATTCCCATTGGGTTCTGGTGGGCTGTAGTGACCATGACTACCCTGGGTTATGAGGATACG
TACCCCCAAACATGGTCAGGCATGCTGGTGGGAGCCCTGTGTGCTCTGGCTGGAGTGCTGACAATAGC
CATGCCAGTGCCTGTCATTGTCAATAATTTTGGAATGTACTACTCCTTGGCAATGGCAAAGCAGAAAC
TTCCAAGGAAAAGAAAGAAGCACATCCCTCCTGCTCCTCAGGCAAGCTCACCTACTTTTTGCAAGACA
GAATTAAATATGGCCTGCAATAGTACACAGAGTGACACATGTCTGGGCAAAGACAATCGACTTCTGGA
ACATAACAGATCAGTGTTATCAGGTGACGACAGTACAGGAAGTGAGCCGCCACTATCACCCCCAGAAA
GGCTCCCCATCAGACGCTCTAGTACCAGAGACAAAAACAGAAGAGGGGAAACATGTTTCCTACTGACG
ACAGGTGATTACACGTGTGCTTCTGATGGAGGGATCAGGAAAGGATATGAAAAATCCCGAAGCTTAAA
CAACATAGCGGGCTTGGCAGGCAATGCTCTGAGGCTCTCTCCAGTAACATCACCCTACAACTCTCCTT
GTCCTCTGAGGCGCTCTCGATCTCCCATCCCATCTATCTTGCTCGAG

NOV9b, 207885588

| | | | |
|---|---|---|---|
| Protein Sequence | SEQ ID NO: 106 | 605 aa | MW at 67228.3kD |

RSPTMGKIENNERVILNVGGTRHETYRSTLKTLPGTRLALLASSEPPGNCSSRGGRASDHPGGGREFF
FDRHPGVFAYVLNYYRTGKLHCPADVCGPLFEEELAFWGIDETDVEPCCWMTYRQHRDAEEALDIFET
PDLIGGDPGDDEDLAAKRLGIEDAAGLGGPDGKSGRWRRLQPRMWALFEDPYSSRAARFIAFASLFFI
LVSITTFCLETHEAFNIVKNKTEPVINGTSVVLQYEIETDPALTYVEGVCVVWFTFEFLVRIVFSPNK
LEFIKNLLNIIDFVAILPFYLEVGLSGLSSKAAKDVLGFLRVVRFVRILRIFKLTRHFVGLRVLGHTL
RASTNEFLLLIIFLALGVLIFATMIYYAERVGAQPNDPSASEHTQFKNIPIGFWWAVVTMTTLGYEDT
YPQTWSGMLVGALCALAGVLTIANPVPVIVNNFGMYYSLAMAKQKLPRKRKKHIPPAPQASSPTFCKT
ELNMACNSTQSDTCLGKDNRLLEHNRSVLSGDDSTGSEPPLSPPERLPIRRSSTRDKNRRGETCFLLT
TGDYTCASDGGIRKGYEKSRSLNNIAGLAGNALRLSPVTSPYNSPCPLRRSRSPIPSILLE

| | | |
|---|---|---|
| NOV9c, CG50249-02 | SEQ ID NO: 107 | 607 bp |

| | | |
|---|---|---|
| DNA Sequence | ORF Start: ATG at 13 | ORF Stop: at 604 |

AGATTTCCCACCATGGGCAAGATCGAGAACAACGAGAGGGTGATCCTCAATGTCGGGGGCACCCGGCA
CGAAACCTACCGCAGCACCCTCAAGACCCTGCCTGGAACACGCCTGGCCCTTCTTGCCTCCTCCGAGC
CCCCAGGCGACTGCTTGACCACAGCGGGCAACTGCAGTTCCCGCGGCGGCAGGGCCAGCGACCATCCC
GGTGGCGGCCGCGAGTTCTTCTTCGACCGGCATCCGGGCGTCTTCGCCTATGTGCTCAATTACTACCG
CACCGGCAAGCTGCACTGTCCCGCAGACGTGTGCGGGCCGCTCTTCGAGGAGGAGCTGGCCTTCTGGG
GCATCGACGAGACCGACGTGGAGCCCTGCTGCTGGATGACCTACCGGCAGCACCGCGACGCCGAGGAG
GCGCTGGACATCTTCGAGACCCCCGACCTCATTGGCGGCGACCCCGGCGACGACGAGGACCTGGCGGC
CAAGAGGCTGGGCATCGAGGACGCGGCGGGGCTCGGGGGCCCCGACGGCAAATCTGGCCGCTGGAGGA
GGCTGCAGCCCCGCATGTGGGCCCTCTTCGACCCCTACTCGTCCAGAGCCGCCAGGCTCG

NOV9c, CG50249-02

| | | | |
|---|---|---|---|
| Protein Sequence | SEQ ID NO: 108 | 197 aa | MW at 21779.0kD |

MGKIENNERVILNVGGTRHETYRSTLKTLPGTRLALLASSEPPGDCLTTAGNCSSRGGRASDHPGGGR
EFFFDRHPGVFAYVLNYYRTGKLHCPADVCGPLFEEELAFWGIDETDVEPCCWMTYRQHRDAEEALDI
FETPDLIGGDPGDDEDLAAKRLGIEDAAGLGGPDGKSGRWRRLQPRMWALFEDPYSSRAAR

| | | |
|---|---|---|
| NOV9d, CG50249-03 | SEQ ID NO: 109 | 1815 bp |

| | | |
|---|---|---|
| DNA Sequence | ORF Start: ATG at 13 | ORF Stop: at 1810 |

AGATCTCCCACCATGGGCAAGATCGAGAACAACGAGAGGGTGATCCTCAATGTCGGGGGCACCCGGCA
CGAAACCTACCGCAGCACCCTCAAGACCCTGCCTGGAACACGCCTGGCCCTTCTTGCCTCCTCCGAGC
CCCCAGGCAACTGCAGTTCCCGCGGCGGCAGGGCCAGCGACCATCCCGGTGGCGGCCGCGAGTTCTTC
TTCGACCGGCACCCGGGCGTCTTCGCCTATGTGCTCAATTACTACCGCACCGGCAAGCTGCACTGCCC
CGCAGACGTGTGCGGGCCGCTCTTCGAGGAGGAGCTGGCCTTCTGGGGCATCGACGAGACCGACGTGG
AGCCCTGCTGCTGGATGACCTACCGGCAGCACCGCGACGCCGAGGAGGCGCTGGACATCTTCGAGACC
CCCGACCTCATTGGCGGCGACCCCGGCGACGACGAGGACCTGGCGGCCAAGAGGCTGGGCATCGAGGA
CGCGGCGGGGCTCGGGGGCCCCGACGGCAAATCTGGCCGCTGGAGGAGGCTGCAGCCCCGCATGTGGG
CCCTCTTCGAAGACCCCTACTCGTCCAGAGCCGCCAGGTTTATTGCTTTTGCTTCTTTATTCTTCATC
CTGGTTTCAATTACAACTTTTTGCCTGGAAACACATGAAGCTTTCAATATTGTTAAAAACAAGACAGA
ACCAGTCATCAATGGCACAAGTGTTGTTCTACAGTATGAAATTGAAACGGATCCTGCCTTGACGTATG
TAGAAGGAGTGTGTGTGGTGTGGTTTACTTTTGAATTTTTAGTCCGTATTGTTTTTTCACCCAATAAA
CTTGAATTCATCAAAAATCTCTTGAATATCATTGACTTTGTGGCCATCCTACCTTTCTACTTAGAGGT
GGGACTCAGTGGGCTGTCATCCAAAGCTGCTAAAGATGTGCTTGGCTTCCTCAGGGTGGTAAGGTTTG
TGAGGATCCTGAGAATTTTCAAGCTCACCCGCCATTTTGTAGGTCTGAGGGTGCTTGGACATACTCTT
CGAGCTAGTACTAATGAATTTTTGCTGCTGATAATTTTCCTGGCTCTAGGAGTTTTGATATTTGCTAC
CATGATCTACTATGCCGAGAGAGTGGGAGCTCAACCTAACGACCCTTCAGCTAGTGAGCACACACAGT
TCAAAAACATTCCCATTGGGTTCTGGTGGGCTGTAGTGACCATGACTACCCTGGGTTATGAGGATACG
TACCCCCAAACATGGTCAGGCATGCTGGTGGGAGCCCTGTGTGCTCTGGCTGGAGTGCTGACAATAGC
CATGCCAGTGCCTGTCATTGTCAATAATTTTGGAATGTACTACTCCTTGGCAATGGCAAAGCAGAAAC
TTCCAAGGAAAAGAAAGAAGCACATCCCTCCTGCTCCTCAGGCAAGCTCACCTACTTTTTGCAAGACA
GAATTAAATATGGCCTGCAATAGTACACAGAGTGACACATGTCTGGGCAAAGACAATCGACTTCTGGA
ACATAACAGATCAGTGTTATCAGGTGACGACAGTACAGGAAGTGAGCCGCCACTATCACCCCCAGAAA
GGCTCCCCATCAGACGCTCTAGTACCAGAGACAAAAACAGAAGAGGGGAAACATGTTTCCTACTGACG
ACAGGTGATTACACGTGTGCTTCTGATGGAGGGATCAGGAAAGGATATGAAAAATCCCGAAGCTTAAA
CAACATAGCGGGCTTGGCAGGCAATGCTCTGAGGCTCTCTCCAGTAACATCACCCTACAACTCTCCTT
GTCCTCTGAGGCGCTCTCGATCTCCCATCCCATCTATCTTGCTCGAG

NOV9d, CG50249-03

| | | | |
|---|---|---|---|
| Protein Sequence | SEQ ID NO: 110 | 599 aa | MW at 66544.6kD |

MGKIENNERVILNVGGTRHETYRSTLKTLPGTRLALLASSEPPGNCSSRGGRASDHPGGGREFFFDRH
PGVFAYVLNYYRTGKLHCPADVCGPLFEEELAFWGIDETDVEPCCWMTYRQHRDAEEALDIFETPDLI
GGDPGDDEDLAAKRLGIEDAAGLGGPDGKSGRWRRLQPRMWALFEDPYSSRAARFIAFASLFFILVSI
TTFCLETHEAFNIVKNKTEPVINGTSVVLQYEIETDPALTYVEGVCVVWFTFEFLVRIVFSPNKLEFI
KNLLNIIDFVAILPFYLEVGLSGLSSKAAKDVLGFLRVVRFVRILRIFKLTRHFVGLRVLGHTLRAST
NEFLLLIIFLALGVLIFATMIYYAERVGAQPNDPSASEHTQFKNIPIGFWWAVVTMTTLGYEDTYPQT
WSGMLVGALCALAGVLTIAMPVPVIVNNFGMYYSLAMAKQKLPRKRKKHIPPAPQASSPTFCKTELNM
ACNSTQSDTCLGKDNRLLEHNRSVLSGDDSTGSEPPLSPPERLPIRRSSTRDKNRRGETCFLLTTGDY
TCASDGGIRKGYEKSRSLNNIAGLAGNALRLSPVTSPYNSPCPLRRSRSPIPSIL

| | | |
|---|---|---|
| NOV9e, CG50249-04 | SEQ ID NO: 111 | 3028 bp |

| | | |
|---|---|---|
| DNA Sequence | ORF Start: ATG at 22 | ORF Stop: TAA at 1861 |

AGTCATGTCTGAGTCACAGAGATGGGCAAGATCGAGAACAACGAGAGGGTGATCCTCAATGTCGGGGG
CACCCGGCACGAAACCTACCGCAGCACCCTCAAGACCCTGCCTGGAACACGCCTGGCCCTTCTTGCCT
CCTCCGAGCCCCCAGGCGACTGCTTGACCACGGCGGGCGACAAGCTGCAGCCGTCGCCGCCTCCACTG
TCGCCGCCGCCGAGAGCGCCCCCGCTGTCCCCCGGGCCAGGCGGCTGCTTCGAGGGCGGCGCGGGCAA
CTGCAGTTCCCGCGGCGGCAGGGCCAGCGACCATCCCGGTGGCGGCCGCGAGTTCTTCTTCGACCGGC
ACCCGGGCGTCTTCGCCTATGTGCTCAATTACTACCGCACCGGCAAGCTGCACTGCCCCGCAGACGTG
TGCGGGCCGCTCTTCGAGGAGGAGCTGGCCTTCTGGGGCATCGACGAGACCGACGTGGAGCCCTGCTG
CTGGATGACCTACCGGCAGCACCGCGACGCCGAGGAGGCGCTGGACATCTTCGAGACCCCCGACCTCA
TTGGCGGCGACCCCGGCGACGACGAGGACCTGGCGGCCAAGAGGCTGGGCATCGAGGACGCGGCGGGG
CTCGGGGGCCCCGACGGCAAATCTGGCCGCTGGAGGAGGCTGCAGCCCCGCATGTGGGCCCTCTTCGA
AGACCCCTACTCGTCCAGAGCCGCCAGGTTTATTGCTTTTGCTTCTTTATTCTTCATCCTGGTTTCAA
TTACAACTTTTTGCCTGGAAACACATGAAGCTTTCAATATTGTTAAAAACAAGACAGAACCAGTCATC
AATGGCACAAGTGTTGTTCTACAGTATGAAATTGAAACGGATCCTGCCTTGACGTATGTAGAAGGAGT
GTGTGTGGTGTGGTTTACTTTTGAATTTTTAGTCCGTATTGTTTTTTCACCCAATAAACTTGAATTCA
TCAAAAATCTCTTGAATATCATTGACTTTGTGGCCATCCTACCTTTCTACTTAGAGGTGGGACTCAGT
GGGCTGTCATCCAAAGCTGCTAAAGATGTGCTTGGCTTCCTCAGGGTGGTAAGGTTTGTGAGGATCCT
GAGAATTTTCAAGCTCACCCGCCATTTTGTAGGTCTGAGGGTGCTTGGACATACTCTTCGAGCTAGTA
CTAATGAATTTTTGCTGCTGATAATTTTCCTGGCTCTAGGAGTTTTGATATTTGCTACCATGATCTAC
TATGCCGAGAGAGTGGGAGCTCAACCTAACGACCCTTCAGCTAGTGAGCACACACAGTTCAAAAACAT
TCCCATTGGGTTCTGGTGGGCTGTAGTGACCATGACTACCCTGGGTTATGGGGATATGTACCCCCAAA
CATGGTCAGGCATGCTGGTGGGAGCCCTGTGTGCTCTGGCTGGAGTGCTGACAATAGCCATGCCAGTG
CCTGTCATTGTCAATAATTTTGGAATGTACTACTCCTTGGCAATGGCAAAGCAGAAACTTCCAAGGAA
AAGAAAGAAGCACATCCCTCCTGCTCCTCAGGCAAGCTCACCTACTTTTTGCAAGACAGAATTAAATA
TGGCCTGCAATAGTACACAGAGTGACACATGTCTGGGCAAAGACAATCGACTTCTGGAACATAACAGA
TCAGTGTTATCAGGTGACGACAGTACAGGAAGTGAGCCGCCACTATCACCCCCAGAAAGGCTCCCCAT
CAGACGCTCTAGTACCAGAGACAAAAACAGAAGAGGGGAAACATGTTTCCTACTGACGACAGGTGATT
ACACGTGTGCTTCTGATGGAGGGATCAGGAAACATAACTGCAAAGAGGTTGTCATTACTGGTTACACG
CAAGCCGAGGCCAGATCTCTTACTTAATGACTTGGGGGAAGGCACAAAACATGAGAGAAAGTGTTGTA
CAGAATTTATCATGGATTATTGACTGCTGAGAAAGGGACAGTGGAATTTAGCCATACAAAGGACTATA
CTGGAAACAGACTTCTGCTGCTGAATGTGCCCTGATGTGACCAGGTTGCACTTGGAAGAGATCCTCCG
CGTCTTCATGAGGCACTTAAAGCTTATAAAAGAACTGCGGCTGGAACTCATCTGGTGCTCCCCATGAG
AGTGCTCTGCTTGTAGACTGGCCAGTGTCCATGAAACAACTGTAAATACCAACATGTGTGCATGGGTC
AACAGTCTTGGCCATTTCTCATCAAAAGAAGCCAAATTCATGATCAACATCTCTGAAGTTTCAAGTAA
GGCCCACACTTCTTTGAATTACTCTTCATGGGCCCACATTAGGTTGTGCTGTGAATTACTTAAGGCAG
TGATACTGATGTAGTATAGTTTTGTCTTAATTTCCCTTATTTCTACTTCTTTGGTTGAATCTATGAAC
TTGATTGTATAATTTTCTTATAAATTACTGATGTAATCAGCTTGTCAATTATGTTGTGAAATTGTTAG
TATTCATTTATCAAAAATGACCTATGTTTAGTCACATATTTGTTTAGTTCTGGGAAATTGTTATAGCT
TAAATGGAACTCACCAACATTATTCATAGTTTAAGTCTTTTATCATTATTACCTCAATTATAAATATT
ACAAAAACATAATTCTGGCAATGAGAGTATTTTTTTATTCAATGATCAAGGAGCAATGTCAGTATATA
GTAGAATATCAATTAAATTATATCCTAAAATGTATATTTTGCATAAAAGAGATATTCTTTAATCAATT
ACTTTTTTGTGAGTTTTGTGGCGAATGAAGCTTGTACGTGTCTTTAAAACTGTTGTAGATGAAACTGT
ATAAGATTTTTACATCTTGCTTAATCAATATTTTCAGAGTCTATTAGTTCCCCTGGGATTCTGAATAT
AACATATAGCCTATTATAAATCCCTGTATCGTGGACCTTTTGTGAACATTTCAAGGCGCATGCACAAC
CTTGATGATAACCAGTGGAAATGTAACTAACTGAAATGAAGAATAAAAGGCAAATGAGCTGGGGATAA
ACTTGAATGTTATCTGATTAAATTACTCAAATTATT

NOV9e, CG50249-04

| | | | |
|---|---|---|---|
| Protein Sequence | SEQ ID NO: 112 | 613 aa | MW at 67598.7kD |

MGKIENNERVILNVGGTRHETYRSTLKTLPGTRLALLASSEPPGDCLTTAGDKLQPSPPPLSPPPRAP
PLSPGPGGCFEGGAGNCSSRGGRASDHPGGGREFFFDRHPGVFAYVLNYYRTGKLHCPADVCGPLFEE
ELAFWGIDETDVEPCCWMTYRQHRDAEEALDIFETPDLIGGDPGDDEDLAAKRLGIEDAAGLGGPDGK
SGRWRRLQPRMWALFEDPYSSRAARFIAFASLFFILVSITTFCLETHEAFNIVKNKTEPVINGTSVVL
QYEIETDPALTYVEGVCVVWFTFEFLVRIVFSPNKLEFIKNLLNIIDFVAILPFYLEVGLSGLSSKAA
KDVLGFLRVVRFVRILRIFKLTRHFVGLRVLGHTLRASTNEFLLLIIFLALGVLIFATMIYYAERVGA
QPNDPSASEHTQFKNIPIGFWWAVVTMTTLGYGDMYPQTWSGMLVGALCALAGVLTIAMPVPVIVNNF
GMYYSLAMAKQKLPRKRKKHIPPAPQASSPTFCKTELNMACNSTQSDTCLGKDNRLLEHNRSVLSGDD
STGSEPPLSPPERLPIRRSSTRDKNRRGETCFLLTTGDYTCASDGGIRKDNCKEVVITGYTQAEARSL
T

A ClustalW comparison of the above protein sequences yields the following sequence alignment shown in Table 9B.

TABLE 9B
Comparison of the NOV9 protein sequences.

| | |
|---|---|
| NOV9a | ----MGKIENNERVILNVGGTRHETYRSTLKTLPGTRLALLASSEPPGDCLTTAGDKLQP |
| NOV9b | RSPTMGKIENNERVILNVGGTRHETYRSTLKLPGTRRLALLSSEPPG------------ |
| NOV9c | ----MGKIENNERVILNVGGTRHETYRSTLKTLPGTRLALLASSEPPGDCLTTA------ |
| NOV9d | ----MGKIENNERVILNVGGTRHETYRSTLKTLPGTRLALLASSEPPG------------ |
| NOV9e | ----MGKIENNERVILNVGGTRHETYRSTLKTLPGTRLALLASSEPPGDCLTTAGDKLQP |
| NOV9a | SPPPLSPPPRAPPLSPGPGGCFEGGAGNCSSRGGRASDHPGGGREFFFDRHPGVFAYVLN |
| NOV9b | ---------------------------NCSSRGGRASDHPGGGREFFFDRHPGVFAYVLN |
| NOV9c | --------------------------GNCSSRGGRASDHPGGGREFFFDRHPGVFAYVLN |
| NOV9d | ---------------------------NCSSRGGRASDHPGGGREFFFDRHPGVFAYVLN |
| NQV9e | SPPPLSPPPRAPPLSPGPGGCFEGGAGNCSSRGGRASDHPGGGREFFFDRHPGVFAYVLN |
| NOV9a | YYRTGKLHCPADVCGPLFEEELAFWGIDETDVEPCCWMTYRQHRDAEEALDIFETPDLIG |
| NOV9b | YYRTGKLHCPADVCGPLFEEELAFWGIDETDVEPCCWMTYRQHRDAEEALDIFETPDLIG |
| NOV9c | YYRTGKLHCPADVCGPLFEEELAFWGIDETDVEPCCWMTYRQHRDAEEALDIFETPDLIG |
| NOV9d | YYRTGKLHCPADVCGPLFEEELAFWGIDETDVEPCCWMTYRQHRDAEEALDIFETPDLIG |
| NOV9e | YYRTGKLHCPADVCGPLFEEELAFWGIDETDVEPCCWMTYRQHRDAEEALDIFETPDLIG |
| NOV9a | GDPGDDEDLAAKRLGIEDAAGLGGPDGKSGRWRRLQPRMWALFEDPYSSRAARFIAFASL |
| NOV9b | GDPGDDEDLAAKRLGIEDAAGLGGPDGKSGRWRRLQPRMWALFEDPYSSRAARFIAFASL |
| NOV9c | GDPGDDEDLAAKRLGIEDAAGLGGPDGKSGRWRRLQPRMWALFEDPYSSRAARFIAFASL |
| NOV9d | GDPGDDEDLAAKRLGIEDAAGLGGPDGKSGRWRRLQPRMWALFEDPYSSRAARFIAFASL |
| NOV9e | GDPGDDEDLAAKRLGIEDAAGLGGPDGKSGRWRRLQPRMWALFEDPYSSRAARFIAFASL |
| NOV9a | FFILVSITTFCLETHEAFNIVKNKTEPVINGTSVVLQYEIETDPALTYVEGVCVVWFTFE |
| NOV9b | FFILVSITTFCLETHEAFNIVKNKTEPVINGTSVVLQYEIETDPALTYVEGVCVVWFTFE |
| NOV9c | ------------------------------------------------------------ |
| NOV9d | FFILVSITTFCLETHEAFNIVKNKTEPVINGTSVVLQYEIETDPALTYVEGVCVVWFTFE |
| NOV9e | FFILVSITTFCLETHEAFNIVKNKTEPVINGTSVVLQYEIETDPALTYVEGVCVVWFTFE |
| NOV9a | FLVRIVFSPNKLEFIKNLLNIIDFVAILPFYLEVGLSGLSSKAAKDVLGFLRVVRFVRIL |
| NOV9b | FLVRIVFSPNKLEFIKNLLNIIDFVAILPFYLEVGLSGLSSKAAKDVLGFLRVVRFVRIL |
| NOV9c | ------------------------------------------------------------ |
| NOV9d | FLVRIVFSPNKLEFIKNLLNIIDFVAILPFYLEVGLSGLSSKAAKDVLGFLRVVRFVRIL |
| NOV9e | FLVRIVFSPNKLEFIKNLLNIIDFVAILPFYLEVGLSGLSSKAAKDVLGFLRVVRFVRIL |
| NOV9a | RIFKLTRHFVGLRVLGHTLRASTNEFLLLIIFLALGVLIFATMIYYAERVGAQPNDPSAS |
| NOV9b | RIFKLTRHFVGLRVLGHTLRASTNEFLLLIIFLALGVLIFATMIYYAERVGAQPNDPSAS |
| NOV9c | ------------------------------------------------------------ |
| NOV9d | RIFKLTRHFVGLRVLGHTLRASTNEFLLLIIFLALGVLIFATMIYYAERVGAQPNDPSAS |
| NOV9e | RIFKLTRHFVGLRVLGHTLRASTNEFLLLIIFLALGVLIFATMIYYAERVGAQPNDPSAS |
| NOV9a | EHTQFKNIPIGFWWAVVTMTTLGYEDTYPQTWSGMLVGALCALAGVLTIAMPVPVIVNNF |
| NOV9b | EHTQFKNIPIGFWWAVVTMTTLGYEDTYPQTWSGMLVGALCALAGVLTIAMPVPVIVNNF |
| NOV9c | ------------------------------------------------------------ |
| NOV9d | EHTQFKNIPIGFWWAVVTMTTLGYEDTYPQTWSGMLVGALCALAGVLTIAMPVPVIVNNF |
| NOV9e | EHTQFKNIPIGFWWAVVTMTTLGYEDTYPQTWSGMLVGALCALAGVLTIAMPVPVIVNNF |
| NOV9a | GMYYSLAMAKQKLPRKRKKHIPPAPQASSPTFCKTELNMACNSTQSDTCLGKDNRLLEHN |
| NOV9b | GMYYSLAMAKQKLPRKRKKHIPPAPQASSPTFCKTELNMACNSTQSDTCLGKDNRLLEHN |
| NOV9c | ------------------------------------------------------------ |
| NOV9d | GMYYSLAMAKQKLPRKRKKHIPPAPQASSPTFCKTELNMACNSTQSDTCLGKDNRLLEHN |
| NOV9e | GMYYSLAMAKQKLPRKRKKHIPPAPQASSPTFCKTELNMACNSTQSDTCLGKDNRLLEHN |
| NOV9a | RSVLSGDDSTGSEPPLSPPERLPIRRSSTRDKNRRGETCFLLTTGDYTCASDGGIRKGYE |
| NOV9b | RSVLSGDDSTGSEPPLSPPERLPIRRSSTRDKNRRGETCFLLTTGDYTCASDGGIRKGYE |
| NOV9c | ------------------------------------------------------------ |
| NOV9d | RSVLSGDDSTGSEPPLSPPERLPIRRSSTRDKNRRGETCFLLTTGDYTCASDGGIRKGYE |
| NOV9e | RSVLSGDDSTGSEPPLSPPERLPIRRSSTRDKNRRGETCFLLTTGDYTCASDGGIRKGYE |
| NOV9a | KSRSLNNIAGLAGNALRLSPVTSPYNSPCPLRRSRSPIPSIL-- |
| NOV9b | KSRSLNNIAGLAGNALRLSPVTSPYNSPCPLRRSRSPIPSILLE |
| NOV9c | -------------------------------------------- |
| NOV9d | KSRSLNNIAGLAGNALRLSPVTSPYNSPCPLRRSRSPIPSIL-- |
| NOV9e | KEVVITGYTQAEARSLT--------------------------- |
| NOV9a | (SEQ ID NO: 104) |
| NOV9b | (SEQ ID NO: 106) |
| NOV9c | (SEQ ID NO: 108) |
| NOV9d | (SEQ ID NO: 110) |
| NOV9e | (SEQ ID NO: 112) |

Further analysis of the NOV9a protein yielded the following properties shown in Table 9C.

TABLE 9C
Protein Sequence Properties NOV9a

```
Signa1P analysis: No Known Signal Sequence Indicated
PSORT II analysis:
PSG: a new signal peptide prediction method
    N-region: length 9; pos. chg 2; neg. chg 2
    H-region: length 8; peak value 4.97
    PSG score: 0.57
GvH: von Heijne's method for signal seq. recognition
    GvH score (threshold: −2.1): −3.54
    possible cleavage site: between 46 and 47
>>> Seems to have no N-terminal signal peptide
ALOM: Klein et al's method for TM region allocation
    Init position for calculation: 1
    Tentative number of TMS(s) for the threshold 0.5: 6
    INTEGRAL Likelihood = −6.90 Transmembrane 230-246
    INTEGRAL Likelihood = −3.24 Transmembrane 287-303
    INTEGRAL Likelihood = −2.23 Transmembrane 314-330
    INTEGRAL Likelihood = −0.16 Transmembrane 343-359
    INTEGRAL Likelihood = −13.00 Transmembrane 382-398
    INTEGRAL Likelihood = −7.01 Transmembrafle 451-467
    PERIPHERAL Likelihood = 3.61 (at 424)
    ALOM score: −13.00 (number of TMSs: 6)
MTOP: Prediction of membrane topology (Hartmann et al.)
    Center position for calculation: 237
    Charge difference: −0.5 C(−0.5) − N( 0.0)
    N >= C: N-terminal side will be inside
>>> membrane topology: type 3a
MITDISC: discrimination of mitochondrial targeting seq
    R content: 0 Hyd Moment (75) : 5.56
    Hyd Moment (95): 3.52G content: 1
    DIE content: 2 S/T content: 0
    Score: −7.90
Gavel: prediction of cleavage sites for mitochondrial preseq
    cleavage site motif not found
NUCDISC: discrimination of nuclear vocalization signals
    pat4: PRKR (4) at 490
    pat4: RKRK (5) at 491
    pat4: KRKK (5) at 492
    pat4: RKKH (3) at 493
    pat7: PRKRKKH (5) at 490
    pat7: PLRRSRS (4) at 626
    bipartite: none
    content of basic residues: 10.7%
    NLS Score: 1.37
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
--------------------------
Final Results (k = 9/23):
    66.7%: endoplasmic reticulum
    22.2%: mitochondrial
    11.1%: nuclear
prediction for CG50249-01 is end (k=9)
```

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| AA014201 | Human transporter and ion | 1 . . . 638 | 638/638 (100%) | 0.0 |
| | channel TRICH-18 - Homo | 1 . . . 638 | 638/638 (100%) | |
| | sapiens, 638 aa. [WO200204520- | | | |
| | A2, 17-JAN-2002] | | | |
| ABP52157 | Human 53763 transporter protein | 1 . . . 638 | 638/638 (100%) | 0.0 |
| | SEQ ID NO:11 - Homo sapiens, | 1 . . . 638 | 638/638 (100%) | |
| | 638 aa. [WO200255701-A2, 18- | | | |
| | JUL-2002] | | | |
| ABG70285 | Human novel polypeptide #1 - | 1 . . . 638 | 638/638 (100%) | 0.0 |
| | Homo sapiens, 638 aa. | 1 . . . 638 | 638/638 (100%) | |
| | [WO200257452-A2, 25-JUL- | | | |
| | 2002] | | | |
| ABB78396 | Longer splice variant of a human | 1 . . . 638 | 638/638 (100%) | 0.0 |
| | voltage-gated potassium channel - | 1 . . . 638 | 638/638 (100%) | |
| | Homo sapiens, 638 aa. | | | |
| | [GB2372503-A, 28-AUG-2002] | | | |
| ABP52167 | Rat voltage gated potassium | 1 . . . 638 | 623/638 (97%) | 0.0 |
| | channel protein KV3.2 SEQ ID | 1 . . . 638 | 625/638 (97%) | |
| | NO:33 - Rattus norvegicus, 638 | | | |
| | aa. [WO200255701-A2, 18-JUL- | | | |
| | 2002] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q96PR1 | Voltage gated potassium channel | 1 . . . 638 | 638/638 (100%) | 0.0 |
| | Kv3 .2b (Potassium voltage-gated | 1 . . . 638 | 638/638 (100%) | |
| | potassium channel subfamily C | | | |
| | member 2) - Homo sapiens | | | |
| | (Human), 638 aa. | | | |
| P22462 | Potassium voltage-gated channel | 1 . . . 638 | 623/638 (97%) | 0.0 |
| | subfamily C member 2 | 1 . . . 638 | 625/638 (97%) | |
| | (Potassium channel Kv3.2) | | | |
| | (KSHIIIA) - Rattus norvegicus | | | |
| | (Rat), 638 aa. | | | |
| AA089503 | Kv3.2d voltage-gated potassium | 1 . . . 593 | 593/593 (100%) | 0.0 |
| | channel - Homo sapiens | 1 . . . 59 | 3593/593 (100%) | |
| | (Human), 629 aa. | | | |
| Q96PR0 | Voltage gated potassium channel | 1 . . . 593 | 593/593 (100%) | 0.0 |
| | Kv3.2a - Homo sapiens (Human), | 1 . . . 593 | 593/593 (100%) | |
| | 613 aa. | | | |
| A39402 | potassium channel protein IIIA | 1 . . . 593 | 578/593 (97%) | 0.0 |
| | form 1, shaker-type - rat, 613 aa. | 1 . . . 593 | 580/593 (97%) | |

PFam analysis indicates that the NOV9a protein contains the domains shown in the Table 9F.

TABLE 9F
Domain Analysis of NOV9a

| | | Identities/ | |
|---|---|---|---|
| | NOV9a | Similarities for | |
| Pfam Domain | Match Region | the Matched Region | Expect Value |
| K_tetra | 9 . . . 156 | 50/161 (31%) | 3.2e-42 |
| | | 121/161 (75%) | |
| ion_trans | 281 . . . 472 | 53/233 (23%) | 6.3e-43 |
| | | 155/233 (67%) | |

### Example 10

The NOV10 clone was analyzed, and the nucleotide and encoded polypeptide sequences are shown in Table 10A.

TABLE 10A
NOV10 Sequence Analysis

| | | |
|---|---|---|
| NOV10a, CG50307-03 | SEQ ID NO: 113 | 1162 bp |

| | | |
|---|---|---|
| DNA Sequence | ORF Start: ATG at 97 | ORF Stop: TGA at 1087 |

ATCTACGGAGTCCCTTTGGCCACATAAGATTGGCCTTAAGAGAAGGACGGAGCCACATACTGCTGACG
GCCCAGAACTGGCAGAGAGAAGGTTGCCATGGCTGCTGTTGACAGTTTCTACCTCTTGTACTGGGAAA
TCGCCAGGTCTTGCAATTGCTATATGGAAGCTCTAGCTTTGGTTGGAGCCTGGTATACGGCCAGAAAA
AGCATCACTGTCATCTGTGACTTTTACAGCCTGATCAGGCTGCATTTTATCCCCCGCCTGGGGAGCAG
AGCAGACTTGATCAAGCAGTATGGAAGATGGGCCGTTGTCAGCGGTGCAACAGATGGGATTGGAAAAG
CCTACGCTGAAGAGTTAGCAAGCCGAGGTCTCAATATAATCCTGATTAGTCGGAACGAGGAGAAGTTG
CAGGTTGTTGCTAAAGACATAGCCGACACGTACAAAGTGGAAACTGATATTATAGTTGCGGACTTCAG
CAGCGGTCGTGAGATCTACCTTCCAATTCGAGAAGCCCTGAAGGACAAAGACGTTGGCATCTTGGTAA
ATAACGTGGGTGTGTTTTATCCCTACCCGCAGTATTTCACTCAGCTGTCCGAGGACAAGCTCTGGGAC
ATCATAAATGTGAACATTGCCGCCGCTAGTTTGATGGTCCATGTTGTGTTACCGGGAATGGTGGAGAG
AAAGAAAGGTGCCATCGTCACGATCTCTTCTGGCTCCTGCTGCAAACCCACTCCTCAGCTGGCTGCAT
TTTCTGCTTCTAAGGCTTATTTAGACCACTTCAGCAGAGCCTTGCAATATGAATATGCCTCTAAAGGA
ATCTTTGTACAGAGTCTAATCCCTTTCTATGTAGCCACCAGCATGACAGCACCCAGCAACTTTCTGCA
CAGGTGCTCGTGGTTGGTGCCTTCGCCAAAAGTCTATGCACATCATGCTGTTTCTACTCTTGGGATTT
CCAAAAGGACCACAGGATATTGGTCCCATTCTATTCAGTTTCTTTTTGCACAGTATATGCCTGAATGG
CTCTGGGTGTGGGGAGCAAATATTCTCAACCGTTCACTACGTAAGGAAGCCTTATCCTGCACAGCCTG
AGTCTGGATGGCCACTTGAGAAGTTTTGCCAACTCCTGGGAACCTCGATATTCTGACATTTGGAAAAA
CACATT

NOV10a, CG50307-03

| | | | |
|---|---|---|---|
| Protein Sequence | SEQ ID NO: 114 | 330 aa | MW at 37031.4kD |

MAAVDSFYLLYWEIARSCNCYMEALALVGAWYTARKSITVICDFYSLIRLHFIPRLGSRADLIKQYGR
WAVVSGATDGIGKAYAEELASRGLNIILISRNEEKLQVVAKDIADTYKVETDIIVADFSSGREIYLPI
REALKDKDVGILVNNVGVFYPYPQYFTQLSEDKLWDIINVNIAAASLMVHVVLPGMVERKKGAIVTIS
SGSCCKPTPQLAAFSASKAYLDHFSRALQYEYASKGIFVQSLIPFYVATSMTAPSNFLHRCSWLVPSP
KVYAHHAVSTLGISKRTTGYWSHSIQFLFAQYMPEWLWVWGANILNRSLRKEALSCTA

| | | |
|---|---|---|
| NOV10b, 275624102 | SEQ ID NO: 115 | 1012 bp |

| | | |
|---|---|---|
| DNA Sequence | ORF Start: at 2 | ORF Stop: end of sequence |

CACCGGATCCACCATGGCTGCTGTTGACAGTTTCTACCTCTTGTACAGGGAAATCGCCAGGTCTTGCA
ATTGCTATATGGAAGCTCTAGCTTTGGTTGGAGCCTGGTATACGGCCAGAAAAAGCATCACTGTCATC
TGTGACTTTTACAGCCTGATCAGGCTGCATTTTATCCCCCGCCTGGGGAGCAGAGCAGACTTGATCAA
GCAGTATGGAAGATGGGCCGTTGTCAGCGGTGCAACAGATGGGATTGGAAAAGCCTACGCTGAAGAGT
TAGCAAGCCGAGGTCTCAATATAATCCTGATTAGTCGGAACGAGGAGAAGTTGCAGGTTGTTGCTAAA
GACATAGCCGACACGTACAAAGTGGAAACTGATATTATAGTTGCGGACTTCAGCAGCGGTCGTGAGAT
CTACCTTCCAATTCGAGAAGCCCTGAAGGACAAAGACGTTGGCATCTTGGTAAATAACGTGGGTGTGT
TTTATCCCTACCCGCAGTATTTCACTCAGCTGTCCGAGGACAAGCTCTGGGACATCATAAATGTGAAC
ATTGCCGCCGCTAGTTTGATGGTCCATGTTGTGTTACCGGGAATGGTGGAGAGAAAGAAAGGTGCCAT
CGTCACGATCTCTTCTGGCTCCTGCTGCAAACCCACTCCTCAGCTGGCTGCATTTTCTGCTTCTAAGG
CTrATTTAGACCACTTCAGCAGAGCCTTGCAATATGAATATGCCTCTAAAGGAATCTTTGTACAGAGT
CTAATCCCTTTCTATGTAGCCACCAGCATGACAGCACCCAGCAACTTTCTGCACAGGTGCTCGTGGTT
GGTGCCTTCGCCAAAAGTCTATGCACATCATGCTGTTTCTACTCTTGGGATTTCCAAAAGGACCACAG
GATATTGGTCCCATTCTATTCAGTTTCTTTTTGCACAGTATATGCCTGAATGGCTCTGGGTGTGGGGA
GCAAATATTCTCAACCGTTCACTACGTAAGGAAGCCTTATCCTGCACAGCCCTCGAGGGC

NOV10b, 275624102

| | | | |
|---|---|---|---|
| Protein Sequence | SEQ ID NO: 116 | 337 aa | MW at 37647.0kD |

TGSTMAAVDSFYLLYREIARSCNCYMEALALVGAWYTARKSITVICDFYSLIRLHFIPRLGSRADLIK
QYGRWAVVSGATDGIGKAYAEELASRGLNIILISRNEEKLQVVAKDIADTYKVETDIIVADFSSGREI
YLPIREALKDKDVGILVNNVGVFYPYPQYFTQLSEDKLWDIINVNIAAASLMVHVVLPGMVERKKGAI
VTISSGSCCKPTPQLAAFSASKAYLDHFSRALQYEYASKGIFVQSLIPFYVATSMTAPSNFLHRCSWL
VPSPKVYAHHAVSTLGISKRTTGYWSHSIQFLFAQYMPEWLWVWGANILNRSLRKEALSCTALEG

| | | |
|---|---|---|
| NOV10c, CG50307-01 | SEQ ID NO: 117 | 1831 bp |

| | | |
|---|---|---|
| DNA Sequence | ORF Start: ATG at 183 | ORF Stop: TGA at 1173 |

ACCGGTTTGGAAGACTTTGCCGGCCTGCAGGACACATGATGACATTGGACCCACCCTCCCCAGCTCGG
AGTCTTTAACTCAGTCACATCTACGGAGTCCCTTTGGCCACATAAGATTGGCCTTAAGAGAAGGACGG
AGCCACATACTGCTGACGGCCCAGAACTGGCAGAGAGAAGGTTGCCATGGCTGCTGTTGACAGTTTCT
ACCTCTTGTACAGGGAAATCGCCAGGTCTTGCAATTGCTATATGGAAGCTCTAGCTTTGGTTGGAGCC
TGGTATACGGCCAGAAAAAGCATCACTGTCATCTGTGACTTTTACAGCCTGATCAGGCTGCATTTTAT
CCCCCGCCTGGGGAGCAGAGCAGACTTGATCAAGCAGTATGGAAGATGGGCCGTTGTCAGCGGTGCAA
CAGATGGGATTGGAAAAGCCTACGCTGAAGAGTTAGCAAGCCGAGGTCTCAATATAATCCTGATTAGT
CGGAACGAGGAGAAGTTGCAGGTTGTTGCTAAAGACATAGCCGACACGTACAAAGTGGAAACTGATAT
TATAGTTGCGGACTTCAGCAGCGGTCGTGAGATCTACCTTCCAATTCGAGAAGCCCTGAAGGACAAAG
ACGTTGGCATCTTGGTAAATAACGTGGGTGTGTTTTATCCCTACCCGCAGTATTTCACTCAGCTGTCC
GAGGACAAGCTCTGGGACATCATAAATGTGAACATTGCCGCCGCTAGTTTGATGGTCCATGTTGTGTT
ACCGGGAATGGTGGAGAGAAAGAAAGGTGCCATCGTCACGATCTCTTCTGGCTCCTGCTGCAAACCCA
CTCCTCAGCTGGCTGCATTTTCTGCTTCTAAGGCTTATTTAGACCACTTCAGCAGAGCCTTGCAATAT
GAATATGCCTCTAAAGGAATCTTTGTACAGAGTCTAATNCCTTTCTATGTAGCCACCAGCATGACAGC
ACCCAGCAACTTTCTGCACAGGTGCTCGTGGTTGGTGCCTTCGCCAAAAGTCTATGCACATCATGCTG
TTTCTACTCTTGGGATTTCCAAAAGGACCACAGGATATTGGTCCCATTCTATTCAGTTTCTTTTTGCA
CAGTATATGCCTGAATGGCTCTGGGTGTGGGGAGCAAATATTCTCAACCGTTCACTACGTAAGGAAGC
CTTATCCTGCACAGCCTGAGTCTGGATGGCCACTTGAGAAGTTTTGCCAACTCCTGGGAACCTCGATA
TTCTGACATTTGGAAAAACACATTTAATTTATCTCCTGTGTTTCATTGCTGATTATTCAGCATACTGT
TGATTCGTCATTTGCAAAACACACATAATACCGTCAGAGTGCTGTGAAAAACCTTAAGGGTGTGTGGA
TGGCACAGGATCAATAATGCCTGAGGCTGATTGACGACATCTACATTTCAGTGCTTTTTCCCTAAGCT
GTTTGAAAGTTACGCTTTTCTGTTGTTCTAGAGCCACAGCAGTCTAATATTGAAATATAATATGATTG
TCAGGTCTTATAATTTCAGATGTTGTTTTTTAAGGGAAATTGACCATTTCACTAGAGGAGTTGTGCTG
GTTTTTACATGTGCATCAAGGAAAGACTACTGGAAAAGTATTTATTTTGGTAACTAAGATTGCTGGCT
ACTATTAGGGACACACTCCGGGCTGTTTGGTATAGCTCTACCTGGTTTGACTATCTGTCATGGAAATG
CTGCCTTCCACTGGTTTTTCCTTTGAGACGGGGTGTGTGCCTGGGTTGTGGGGCCCTTGGGCCCCTTT
TTTTTGGTGCCCCTTCTTCCACCCACTTTCGGCCCGCGGGCCCCCTGGCGCTCTGGGTTTCCC

NOV10c, CG50307-01

| | | | |
|---|---|---|---|
| Protein Sequence | SEQ ID NO: 118 | 330 aa | MW at 36888.2kD |

MAAVDSFYLLYREIARSCNCYMEALALVGAWYTARKSITVICDFYSLIRLHFIPRLGSRADLIKQYGR
WAVVSGATDGIGKAYAEELASRGLNIILISRNEEKLQVVAKDIADTYKVETDIIVADFSSGREIYLPI
REALKDKDVGILVNNVGVFYPYPQYFTQLSEDKLWDIINVNIAAASLMVHVVLPGMVERKKGAIVTIS
SGSCCKPTPQLAAFSASKAYLDHFSRALQYEYASKGIFVQSLXPFYVATSMTAPSNFLHRCSWLVPSP
KVYAHHAVSTLGISKRTTGYWSHSIQFLFAQYMPEWLWVWGANILNRSLRKEALSCTA

| | | |
|---|---|---|
| NOV10d, CG50307-02 | SEQ ID NO: 119 | 1152 bp |

| | | |
|---|---|---|
| DNA Sequence | ORF Start: ATG at 97 | ORF Stop: TGA at 1087 |

ATCTACGGAGTCCCTTTGGCCACATAAGATTGGCCTTAAGAGAAGGACGGAGCCACATACTGCTGACG
GCCCAGAACTGGCAGAGAGAAGGTTGCCATGGCTGCTGTTGACAGTTTCTACCTCTTGTACAGGGAAA
TCGCCAGGTCTTGCAATTGCTATATGGAAGCTCTAGCTTTGGTTGGAGCCTGGTATACGGCCAGAAAA
AGCATCACTGTCATCTGTGACTTTTACAGCCTGATCAGGCTGCATTTTATCCCCCGCCTGGGGAGCAG
AGCAGACTTGATCAAGCAGTATGGAAGATGGGCCGTTGTCAGCGGTGCAACAGATGGGATTGGAAAAG
CCTACGCTGAAGAGTTAGCAAGCCGAGGTCTCAATATAATCCTGATTAGTCGGAACGAGGAGAAGTTG
CAGGTTGTTGCTAAAGACATAGCCGACACGTACAAAGTGGAAACTGATATTATAGTTGCGGACTTCAG
CAGCGGTCGTGAGATCTACCTTCCAATTCGAGAAGCCCTGAAGGACAAAGACGTTGGCATCTTGGTAA
ATAACGTGGGTGTGTTTTATCCCTACCCGCAGTATTTCACTCAGCTGTCCGAGGACAAGCTCTGGGAC
ATCATAAATGTGAACATTGCCGCCGCTAGTTTGATGGTCCATGTTGTGTTACCGGGAATGGTGGAGAG
AAAGAAAGGTGCCATCGTCACGATCTCTTCTGGCTCCTGCTGCAAACCCACTCCTCAGCTGGCTGCAT
TTTCTGCTTCTAAGGCTTATTTAGACCACTTCAGCAGAGCCTTGCAATATGAATATGCCTCTAAAGGA
ATCTTTGTACAGAGTCTAATCCCTTTCTATGTAGCCACCAGCATGACAGCACCCAGCAACTTTCTGCA
CAGGTGCTCGTGGTTGGTGCCTTCGCCAAAAGTCTATGCACATCATGCTGTTTCTACTCTTGGGATTT
CCAAAAGGACCACAGGATATTGGTCCCATTCTATTCAGTTTCTTTTTGCACAGTATATGCCTGAATGG
CTCTGGGTGTGGGGAGCAAATATTCTCAACCGTTCACTACGTAAGGAAGCCTTATGCTGCACAGCCTG
AGTCTGGATGGCCACTTGAGAAGTTTTGCCAACTCCTGGGAACCTCGATATTCTGACATTTGGA

NOV10d, CG50307-02

| | | | |
|---|---|---|---|
| Protein Sequence | SEQ ID NO: 120 | 330 aa | MW at 37017.4kD |

MAAVDSFYLLYREIARSCNCYMEALALVGAWYTARKSITVICDFYSLIRLHFIPRLGSRADLIKQYGR
WAVVSGATDGIGKAYAEELASRGLMIILISRNEEKLQVVAKDIADTYKVETDIIVADFSSGREIYLPI
REALKDKDVGILVNNVGVFYPYPQYFTQLSEDKLWDIINVNIAAASLMVHVVLPGMVERKKGAIVTIS
SGSCCKPTPQLAAFSASKAYLDHFSRALQYEYASKGIFVQSLIPFYVATSMTAPSNFLHRCSWLVPSP
KVYAHHAVSTLGISKRTTGYWSHSIQFLFAQYMPEWLWVWGANILNRSLRKEALCCTA

| | |
|---|---|
| SEQ ID NO: 121 | 1162bp |

| | | |
|---|---|---|
| NOV10e, SNP13375811 of | ORF Start: ATG at 97 | ORF Stop: TGA at 1087 |

| | | |
|---|---|---|
| CG50307-03, DNA Sequence | SNP Pos: 1076 | SNP Change: C to G |

ATCTACGGAGTCCCTTTGGCCACATAAGATTGGCCTTAAGAGAAGGACGGAGCCACATACTGCTGACG
GCCCAGAACTGGCAGAGAGAAGGTTGCCATGGCTGCTGTTGACAGTTTCTACCTCTTGTACTGGGAAA
TCGCCAGGTCTTGCAATTGCTATATGGAAGCTCTAGCTTTGGTTGGAGCCTGGTATACGGCCAGAAAA
AGCATCACTGTCATCTGTGACTTTTACAGCCTGATCAGGCTGCATTTTATCCCCCGCCTGGGGAGCAG
AGCAGACTTGATCAAGCAGTATGGAAGATGGGCCGTTGTCAGCGGTGCAACAGATGGGATTGGAAAAG
CCTACGCTGAAGAGTTAGCAAGCCGAGGTCTCAATATAATCCTGATTAGTCGGAACGAGGAGAAGTTG
CAGGTTGTTGCTAAAGACATAGCCGACACGTACAAAGTGGAAACTGATATTATAGTTGCGGACTTCAG
CAGCGGTCGTGAGATCTACCTTCCAATTCGAGAAGCCCTGAAGGACAAAGACGTTGGCATCFIGGTAA
ATAACGTGGGTGTGTTTTATCCCTACCCGCAGTATTTCACTCAGCTGTCCGAGGACAAGCTCTGGGAC
ATCATAAATGTGAACATTGCCGCCGCTAGTTTGATGGTCCATGTTGTGTTACCGGGAATGGTGGAGAG
AAAGAAAGGTGCCATCGTCACGATCTCTTCTGGCTCCTGCTGCAAACCCACTCCTCAGCTGGCTGCAT
TTTCTGCTTCTAAGGCTTATTTAGACCACTTCAGCAGAGCCTTGCAATATGAATATGCCTCTAAAGGA
ATCTTTGTACAGAGTCTAATCCCTTTCTATGTAGCCACCAGCATGACAGCACCCAGCAACTTTCTGCA
CAGGTGCTCGTGGTTGGTGCCTTCGCCAAAAGTCTATGCACATCATGCTGTTTCTACTCTTGGGATTT
CCAAAAGGACCACAGGATATTGGTCCCATTCTATTCAGTTTCTTTTTGCACAGTATATGCCTGAATGG
CTCTGGGTGTGGGGAGCAAATATTCTCAACCGTTCACTACGTAAGGAAGCCTTATGCTGCACAGCCTG
AGTCTGGATGGCCACTTGAGAAGTTTTGCCAACTCCTGGGAACCTCGATATTCTGACATTTGGAAAAA
CACATT

| | | |
|---|---|---|
| NOV10e, SNP13375811 of | SEQ ID NO: 122 | MW at 37047.4kD |

| | | | |
|---|---|---|---|
| CG50307-03, Protein Sequence | SNP Pos: 327 | 330 aa | SNP Change: Ser to Cys |

MAAVDSFYLLYWEIARSCNCYMEALALVGAWYTARKSITVICDFYSLIRLHFIPRLGSRADLIKQYGR
WAVVSGATDGIGKAYAEELASRGLNIILISRNEEKLQVVAKDIADTYKVETDIIVADFSSGREIYLPI
REALKDKDVGILVNNVGVFYPYPQYFTQLSEDKLWDIINVNIAAASLMVHVVLPGMVERKKGAIVTIS
SGSCCKPTPQLAAFSASKAYLDHFSRALQYEYASKGIFVQSLIPFYVATSMTAPSNFLHRCSWLVPSP
KVYAHHAVSTLGISKRTTGYWSHSIQFLFAQYMPEWLWVWGANILNRSLRKEALCCTA

A ClustalW comparison of the above protein sequences yields the following sequence alignment shown in Table 10B.

TABLE 10B
Comparison of the NOV10 protein sequences.

| | |
|---|---|
| NOV10a | ----MAAVDSFYLLYWEIARSCNCYMEALALVGAWYTARKSITVICDFYSLIRLHFIPRL |
| NOV10b | TGSTMAAVDSFYLLYREIARSCNCYMEALALVGAWYTARKSITVICDFYSLIRLHFIPRL |
| NOV10c | ----MAAVDSFYLLYREIARSCNCYMEALALVGAWYTARKSITVICDFYSLIRLHFIPRL |
| NOV10d | ----MAAVDSFYLLYREIARSCNCYMEALALVGAWYTARKSITVICDFYSLIRLHFIPRL |
| NOV10a | GSRADLIKQYGRWAVVSGATDGIGKAYAEELASRGLNIILISRNEEKLQVVAKDIADTYK |
| NOV10b | GSRADLIKQYGRWAVVSGATDGIGKAYAEELASRGLNIILISRNEEKLQVVAKDIADTYK |
| NOV10c | GSRADLIKQYGRWAVVSGATDGIGKAYAEELASRGLNIILISRNEEKLQVVAKDIADTYK |
| NOV10d | GSRADLIKQYGRWAVVSGATDGIGKAYAEELASRGLNIILISRNEEKLQVVAKDIADTYK |
| NOV10a | VETDIIVADFSSGREIYLPIREALKDKDVGILVNNVGVFYPYPQYFTQLSEDKLWDIINV |
| NOV10b | VETDIIVADFSSGREIYLPIREALKDKDVGILVNNVGVFYPYPQYFTQLSEDKLWDIINV |
| NOV10c | VETDIIVADFSSGREIYLPIREALKDKDVGILVNNVGVFYPYPQYFTQLSEDKLWDIINV |
| NOV10d | VETDIIVADFSSGREIYLPIREALKDKDVGILVNNVGVFYPYPQYFTQLSEDKLWDIINV |
| NOV10a | NIAAASLMVHVVLPGMVERKKGAIVTISSGSCCKPTPQLAAFSASKAYLDHFSRALQYEY |
| NOV10b | NIAAASLMVHVVLPGMVERKKGAIVTISSGSCCKPTPQLAAFSASKAYLDHFSRALQYEY |
| NOV10c | NIAAASLMVHVVLPGMVERKKGAIVTISSGSCCKPTPQLAAFSASKAYLDHFSRALQYEY |
| NOV10d | NIAAASLMVHVVLPGMVERKKGAIVTISSGSCCKPTPQLAAFSASKAYLDHFSRALQYEY |
| NOV10a | ASKGIFVQSLIPFYVATSMTAPSNFLHRCSWLVPSPKVYAHHAVSTLGISKRTTGYWSHS |
| NOV10b | ASKGIFVQSLIPFYVATSMTAPSNFLHRCSWLVPSPKVYAHHAVSTLGISKRTTGYWSHS |
| NOV10c | ASKGIFVQSLIPFYVATSMTAPSNFLHRCSWLVPSPKVYAHHAVSTLGISKRTTGYWSHS |
| NOV10d | ASKGIFVQSLIPFYVATSMTAPSNFLHRCSWLVPSPKVYAHHAVSTLGISKRTTGYWSHS |
| NOV10a | IQFLFAQYMPEWLWVWGANILNRSLRKEALSCTA--- |
| NOV10b | IQFLFAQYMPEWLWVWGANILNRSLRKEALSCTALEG |
| NOV10c | IQFLFAQYMPEWLWVWGANILNRSLRKEALSCTA--- |
| NOV10d | IQFLFAQYMPEWLWVWGANILNRSLRKEALCCTA--- |
| NOV10a | (SEQ ID NO: 114) |
| NOV10b | (SEQ ID NO: 116) |
| NOV10c | (SEQ ID NO: 118) |
| NOV10d | (SEQ ID NO: 120) |

Further analysis of the NOV10a protein yielded the following properties shown in Table 10C.

TABLE 10C
Protein Sequence Properties NOV10a

```
SignalP analysis: No Known Signal Sequence Indicated
PSORT II analysis:
PSG: a new signal peptide prediction method
    N-region: length 5; pos. chg 0; neg. chg 1
    H-region: length 7; peak value 0.00
    PSG score: −4.40
GvH: von Heijne's method for signal seq. recognition
    GvH score (threshold: −2.1): −6.54
    possible cleavage site: between 58 and 59
>>> Seems to have no N-terminal signal peptide
ALOM: Klein et al's method for TM region allocation
    mit position for calculation: 1
    Tentative number of TMS(s) for the threshold 0.5: 1
    Number of TMS(s) for threshold 0.5: 1
    INTEGRAL Likelihood = −3.88 Transmembrane 173-189
    PERIPHERAL Likelihood = 2.01 (at 37)
    ALOM score: −3.88 (number of TMSs: 1)
MTOP: Prediction of membrane topology (Hartmann et al.)
    Center position for calculation: 180
    Charge difference: 4.5 C( 2.5) − N(−2.0)
    C > N: C-terminal side will be inside
>>> membrane topology: type lb (cytoplasmic tail 173 to 330)
MITDISC: discrimination of mitochondrial targeting seq
    R content: 0 Hyd Moment(75) : 6.52
    Hyd Moment(95) : 5.94 G content: 0
    D/E content: 2 S/T content: 1
    Score: −6.71
Gavel: prediction of cleavage sites for mitochondrial preseq
    cleavage site motif not found
NUCDISC: discrimination of nuclear localization signals
    pat4: none
    pat7: none
    bipartite: none
    content of basic residues: 10.0%
    NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 94.1
COIL: Lupas's algorithm to detect coiled-coil regions
    total: 0 residues
--------------------------
Final Results (k = 9/23)
    34.8%: nuclear
    21.7%: mitochondrial
    21.7%: cytoplasmic
    8.7%: vesicles of secretory system
    4.3%: vacuolar
    4.3%: peroxisomal
    4.3%: endoplasmic reticulum
prediction for CG50307-03 is nuc (k=23)
```

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identifies/ | |
|---|---|---|---|---|
| | | Residues/ | Similarities for | |
| Genesq | Protein/Organism/Length | Match | the Matched | Expect |
| Identifier | [Patent #,Date] | Residues | Region | Value |
| AAE21050 | Human ding metabolising | 1 . . . 330 | 329/330 (99%) | 0.0 |
| | enzyme (DME-8) protein - Homo | 1 . . . 330 | 329/330 (99%) | |
| | sapiens, 330 aa. [WO200212467- | | | |
| | A2, 14-FEB-2002] | | | |
| AAE21521 | Human dehydrogenase DHDR-5 | 1 . . . 330 | 329/330 (99%) | 0.0 |
| | protein - Homo sapiens, 330 aa. | 1 . . . 330 | 329/330 (99%) | |
| | [WO200216562-A2, 28-FEB- | | | |
| | 2002] | | | |
| ABJ10602 | Human novel protein NOV10a | 1 . . . 330 | 329/330 (99%) | 0.0 |
| | SEQ ID NO: 32 - Homo sapiens, | 1 . . . 330 | 29/330 (99%) | |
| | 330 aa. [WO200259315-A2, 01- | | | |
| | AUG-2002] | | | |
| AAM41389 | Human polypeptide SEQ ID NO | 1 . . . 330 | 329/330 (99%) | 0.0 |
| | 6320 - Homo sapiens, 340 aa. | 11 . . . 340 | 329/330 (99%) | |
| | [WO200153312-A1, 26-JUL- | | | |
| | 2001] | | | |
| AAM39603 | Human polypeptide SEQ ID NO | 1 . . . 330 | 329/330 (99%) | 0.0 |
| | 2748 - Homo sapiens, 330 aa. | 1 . . . 330 | 329/330 (99%) | |
| | [WO200153312-A1, 26-JUL- | | | |
| | 2001] | | | |

In a BLAST search of public sequence databases, the NOV10a protein was found to have homology to the proteins shown in the BLASTP data in Table 10E.

TABLE 10E
Public BLASTP Results for NOV10a

| | | | Identities/ | |
|---|---|---|---|---|
| | | NOV10a | Similarities | |
| Protein | | Residues/ | for the | |
| Accession | | Match | Matched | Expect |
| Number | Protein/Organism/Length | Residues | Portion | Value |
| CAD28991 | Sequence 1 from Patent | 1 . . . 330 | 329/330 (99%) | 0.0 |
| | WO0216562 - Homo sapiens | 1 . . . 330 | 329/330 (99%) | |
| | (Human), 330 aa. | | | |
| Q8NC98 | Hypothetical protein FLJ90397 - | 1 . . . 330 | 328/330 (99%) | 0.0 |
| | Homo sapiens (Human), 330 | 1 . . . 330 | 328/330 (99%) | |
| | aa. | | | |
| Q9BY22 | Steroid dehydrogenase-like | 22 . . . 330 | 309/309 (100%) | e−179 |
| | protein - Homo sapiens | 1 . . . 309 | 309/309 (100%) | |
| | (Human), 309 aa. | | | |
| Q8WVE5 | Steroid dehydrogenase-like - | 22 . . . 330 | 308/309 (99%) | e−178 |
| | Homo sapiens (Human), 309 | 1 . . . 309 | 308/309 (99%) | |
| | aa. | | | |
| Q8BTX9 | Steroid dehydrogenase-like | 1 . . . 330 | 289/330 (87%) | e−173 |
| | protein homolog - Mus | 1 . . . 330 | 310/330 (93%) | |
| | musculus (Mouse), 330 aa. | | | |

PFam analysis indicates that the NOV10a protein contains the domains shown in the Table 10F.

TABLE 10F
Domain Analysis of NOV10a

| | NOV10a | Identities/ | |
|---|---|---|---|
| | Match | Similarities | |
| Pfam Domain | Region | for the Matched Region | Expect Value |
| adh_short | 66 . . . 306 | 73/275 (27%) | 8e−24 |
| | | 166/275 (60%) | |

### Example 11

The NOV11 clone was analyzed, and the nucleotide and encoded polypeptide sequences are shown in Table 11A.

TABLE 11A
NOV11 Sequence Analysis

| | | |
|---|---|---|
| NOV11a, CG50315-01 | SEQ ID NO: 123 | 938 bp |

| | | |
|---|---|---|
| DNA Sequence | ORF Start: at 1 | ORF Stop: TAG at 931 |

AATCTCACCAGAGTAACCGAATTCATTCTCATGGGCTTTATGGACCACCCCAAATTGGAGATTCCCCT
CTTTCTGGTGTTTCTGAGTTTCTACCTAGTCACCCTTCTTGGGAATGTGGGGATGATTATGTTAATCC
AAGTAGATGTCAAACTCTACACCCCAATGTACTTCTTCCTGAGCCACCTCTCCCTGCTGGATGCCTGT
TACACCTCAGTCATCACCCCTCAGATCCTAGCCACATTGGCCACAGGCAAAACGGTCATCTCCTACGG
CCACTGTGCTGCCCAGTTCTTTTTATTCACCATCTGTGCAGGCACAGAGTGCTTTCTGCTGGCAGTGA
TGGCCTATGATCGCTATGCTGCCATTCGCAACCCACTGCTCTATACCGTGGCCATGAATCCCAGGCTC
TACTGGAGCCTGGTGGTAGGAGCCTATGTCTGTGGGGTGTCAGGAGCCATCCTGCGTACCACTTGCAC
CTTCACCCTCTCCTTCTGTAAGGACAATCAAATAAACTTCTTCTTCTGTGACCTCCCACCCCTGCTGA
AGCTTGCCTGCAGTGACACAGCAAACATCGAGATTGTCATCATCTTCTTTGGCAATTTTGTGATTTTG
GCCAATGCCTCCGTCATCCTGATTTCCTATCTGCTCATCATCAAGACCATTTTGAAAGTGAAGTCTTC
AGGTGGCAGGGCCAAGACTTTCTCCACATGTGCCTCTCACATCACTGCTGTGGCCCTTTTCTTTGGAG
CCCTTATCTTCATGTATCTGCAAAGTGGCTCAGGCAAATCTCTGGAGGAAGACAAAGTCGTGTCTGTC
TTCTATACAGTGGTCATCCCCATGCTGAACCCTCTGATCTACAGCTTAAGAAACAAAGATGTAAAAGA
CGCCTTCAGAAAGGTCGCTAGGAGACTCCAGGTGTCCCTGAGCATGTAGATTTA

NOV11a, CG50315-01

| | | | |
|---|---|---|---|
| Protein Sequence | SEQ ID NO: 124 | 310 aa | MW at 34522.7kD |

NLTRVTEFILMGFMDHPKLEIPLFLVFLSFYLVTLLGNVGMIMLIQVDVKLYTPMYFFLSHLSLLDAC
YTSVITPQILAThATGKTVISYGHCAAQFFLFTICAGTECFLLAVMAYDRYAAIRNPLLYTVAMNPRL
YWSLVVGAYVCGVSGAILRTTCTFTLSFCKDNQINFFFCDLPPLLKLACSDTANIEIVIIFFGNFVIL
ANASVILISYLLIIKTILKVKSSGGRAKTFSTCASHITAVALFFGALIFMYLQSGSGKSLEEDKVVSV
FYTVVIPMLNPLIYSLRNKDVKDAFRKVARRLQVSLSM

| | | |
|---|---|---|
| NOV11b, 207580272 | SEQ ID NO: 125 | 822 bp |

| | | |
|---|---|---|
| DNA Sequence | ORF Start: at 1 | ORF Stop: end of sequence |

GGATCCATGATTATGTTAATCCAAGTAGATGTCAAACTCTACACCCCAATGTACTTCTTCCTGAGCCA
CCTCTCCCTGCTGGATGCCTGTTACACCTCAGTCATCACCCCTCAGATCCTAGCCACATTGGCCACAG
GCAAAACGGTCATCTCCTACGGCCACTGTGCTGCCCAGTTCTTTTTATTCACCATCTGTGCAGGCACA
GAGTGCTTTCTGCTGGCAGTGATGGCCTATGATCGCTATGCTGCCATTCGCAACCCACTGCTCTATAC
CGTGGCCATGAATCCCAGGCTCTACTGGAGCCTGGTGGTAGGAGCCTATGTCTGTGGGGTGTCAGGAG
CCATCCTGCGTACCACTTGCACCTTCACCCTCTCCTTCTGTAAGGACAATCAAATAAACTTCTTCTTC
TGTGACCTCCCACCCCTGCTGAAGCTTGCCTGCAGTGACACAGCAAACATCGAGATTGTCATCATCTT
CTTTGGCAATTTTGTGATTTTGGCCAATGCCTCCGTCATCCTGATTTCCTATCTGCTCATCATCAAGA
CCATTTTGAAAGTGAAGTCTTCAGGTGGCAGGGCCAAGACTTTCTCCACATGTGCCTCTCACATCACT
GCTGTGGCCCTTTTCTTTGGAGCCCTTATCTTCATGTATCTGCAAAGTGGCTCAGGCAAATCTCTGGA
GGAAGACAAAGTCGTGTCTGTCTTCTATACAGTGGTCATCCCCATGCTGAACCCTCTGATCTACAGCT
TAAGAAACAAAGATGTAAAAGACGCCTTCAGAAAGGTCGCTAGGAGACTCCAGGTGTTCCTGAGCATG
CTCGAG

NOV11b, 207580272

| | | | |
|---|---|---|---|
| Protein Sequence | SEQ ID NO: 126 | 274 aa | MW at 30387.7kD |

GSMIMLIQVDVKLYTPMYFFLSHLSLLDACYTSVITPQILATLATGKTVISYGHCAAQFFLFTICAGT
ECFLLAVMAYDRYAAIRNPLLYTVAMNPRLYWSLVVGAYVCGVSGAILRTTCTFTLSFCKDNQINFFF
CDLPPLLKLACSDTANIEIVIIFFGNFVILANASVILISYLLIIKTILKVKSSGGRAKTFSTCASHIT
AVALFFGALIFMYLQSGSGKSLEEDKVVSVFYTVVIPMLNPLIYSLRNKDVKDAFRKVARRLQVFLSM
LE

| | | |
|---|---|---|
| NOV11c, 314411778 | SEQ ID NO: 127 | 964 bp |

| | | |
|---|---|---|
| DNA Sequence | ORF Start: at 2 | ORF Stop: end of sequence |

CACCGGATCCACCATGGCCAAGAATAATCTCACCAGAGTAACCGAATTCATTCTCATGGGCTTTATGG
ACCACCCCAAATTGGAGATTCCCCTCTTTCTGGTGTTTCTGAGTTTCTACCTAGTCACCCTTCTTGGG
AATGTGGGGATGATTATGTTAATCCAAGTAGATGTCAAACTCTACACCCCAATGTACTTCTTCCTGAG
CCACCTCTCCCTGCTGGATGCCTGTTACACCTCAGTCATCACCCCTCAGATCCTAGCCACATTGGCCA
CAGGCAAAACGGTCATCTCCTACGGCCACTGTGCTGCCCAGTTCTTTTTATTCACCATCTGTGCAGGC
ACAGAGTGCTTTCTGCTGGCAGTGATGGCCTATGATCGCTATGCTGCCATTCGCAACCCACTGCTCTA
TACCGTGGCCATGAATCCCAGGCTCTGCTGGAGCCTGGTGGTAGGAGCCTATGTCTGTGGGGTGTCAG
GAGCCATCCTGCGTACCACTTGCACCTTCACCCTCTCCTTCTGTAAGGACAATCAAATAAACTTCTTC
TTCTGTGACCTCCCACCCCTGCTGAAGCTTGCCTGCAGTGACACAGCAAACATCGAGATTGTCATCAT
CTTCTTTGGCAATTTTGTGATTTTGGCCAATGCCTCCGTCATCCTGATTTCCTATCTGCTCATCATCA
AGACCATTTTGAAAGTGAAGTCTTCAGGTGGCAGGGCCAAGACTTTCTCCACATGTGCCTCTCACATC
ACTGCTGTGGCCCTTTTCTTTGGAGCCCTTATCTTCATGTATCTGCAAAGTGGCTCAGGCAAATCTCT
GGAGGAAGACAAAGTCGTGTCTGTCTTCTATACAGTGGTCATCCCCATGCTGAACCCTCTGATCTACA
GCTTAAGAAACAAAGATGTAAAAGACGCCTTCAGAAAGGTCGCTAGGAGACTCCAGGTGTCCCTGAGC
ATGCTCGAGGGC

NOV11c, 314411778

| | | | |
|---|---|---|---|
| Protein Sequence | SEQ ID NO: 128 | 321 aa | MW at 35552.9kD |

TGSTMAKNNLTRVTEFILMGFMDNPKLEIPLFLVFLSFYLVTLLGNVGMIMLIQVDVKLYTPMYFFLS
HLSLLDACYTSVITPQILATLATGKTVISYGHCAAQFFLFTICAGTECFLLAVMAYDRYAAIRNPLLY
TVANNPRLCWSLVVGAYVCGVSGAILRTTCTFTLSFCKDNQINFFFCDLPPLLKLACSDTANIEIVII
FFGNFVILANASVILISYLLIIKTILKVKSSGGRAKTFSTCASHITAVALFFGALIFMYLQSGSGKSL
EEDKVVSVFYTVVIPMLNPLIYSLRNKDVKDAFRKVARRLQVSLSMLEG

| | | |
|---|---|---|
| NOV11d, CG50315-02 | SEQ ID NO: 129 | 968 bp |

| | | |
|---|---|---|
| DNA Sequence | ORF Start: ATG at 17 | ORF Stop: TAG at 959 |

AGTCCACATTGTCTCCATGGCCAAGAATAATCTCACCAGAGTAACCGAATTCATTCTCATGGGCTTTA
TGGACCACCCCAAATTGGAGATTCCCCTCTTTCTGGTGTTTCTGAGTTTCTACCTAGTCACCCTTCTT
GGGAATGTGGGGATGATTATGTTAATCCAAGTAGATGTCAAACTCTACACCCCAATGTACTTCTTCCT
GAGCCACCTCTCCCTGCTGGATGCCTGTTACACCTCAGTCATCACCCCTCAGATCCTAGCCACATTGG
CCACAGGCAAAACGGTCATCTCCTACGGCCACTGTGCTGCCCAGTTCTTTTTATTCACCATCTGTGCA
GGCACAGAGTGCTTTCTGCTGGCAGTGATGGCCTATGATCGCTATGCTGCCATTCGCAACCCAOTGCT
CTATACCGTGGCCATGAATCCCAGGCTCTGCTGGAGCCTGGTGGTAGGAGCCTATGTCTGTGGGGTGT
CAGGAGCCATCCTGCGTACCACTTGCACCTTCACCCTCTCCTTCTGTAAGGACAATCAAATAAACTTC
TTCTTCTGTGACCTCCCACCCCTGCTGAAGCTTGCCTGCAGTGACACAGCAAACATCGAGATTGTCAT
CATCTTCTTTGGCAATTTTGTGATTTTGGCCAATGCCTCCGTCATCCTGATTTCCTATCTGCTCATCA
TCAAGACCATTTTGAAAGTGAAGTCTTCAGGTGGCAGGGCCAAGACTTTCTCCACATGTGCCTCTCAC
ATCACTGCTGTGGCCCTTTTCTTTGGAGCCCTTATCTTCATGTATCTGCAAAGTGGCTCAGGCAAATC
TCTGGAGGAAGACAAAGTCGTGTCTGTCTTCTATACAGTGGTCATCCCCATGCTGAACCCTCTGATCT
ACAGCTTAAGAAACAAAGATGTAAAAGACGCCTTCAGAAAGGTCGCTAGGAGACTCCAGGTGTCCCTG
AGCATGTAGATCTAAG

NOV11d, CG50315-02

| | | | |
|---|---|---|---|
| Protein Sequence | SEQ ID NO: 130 | 314 aa | MW at 34907.2kD |

MAKNNLTRVTEFILMGFMDHPKLEIPLFLVFLSFYLVTLLGNVGMIMLIQVDVKLYTPMYFFLSHLSL
LDACYTSVITPQILATLATGKTVISYGHCAAQFFLFTICAGTECFLLAVMAYDRYAAIRNPLLYTVAM
NPRLCWSLVVGAYVCGVSGAILRTTCTFTLSFCKDNQINFFFCDLPPLLKLACSDTANIEIVIIFFGN
FVILANASVILISYLLIIKTILKVKSSGGRAKTFSTCASHITAVALFFGALIFMYLQSGSGKSLEEDK
VVSVFYTVVIPMLNPLIYSLRNKDVKDAFRKVARRLQVSLSM

| | | |
|---|---|---|
| NOV11e, CG50315-03 | SEQ ID NO: 131 | 822 bp |

| | | |
|---|---|---|
| DNA Sequence | ORF Start: ATG at 7 | ORF Stop: at 817 |

GGATCCATGATTATGTTAATCCAAGTAGATGTCAAACTCTACACCCCAATGTACTTCTTCCTGAGCCA
CCTCTCCCTGCTGGATGCCTGTTACACCTCAGTCATCACCCCTCAGATCCTAGCCACATTGGCCACAG
GCAAAACGGTCATCTCCTACGGCCACTGTGCTGCCCAGTTCTTTTTATTCACCATCTGTGCAGGCACA
GAGTGCTTTCTGCTGGCAGTGATGGCCTATGATCGCTATGCTGCCATTCGCAACCCACTGCTCTATAC
CGTGGCCATGAATCCCAGGCTCTACTGGAGCCTGGTGGTAGGAGCCTATGTCTGTGGGGTGTCAGGAG
CCATCCTGCGTACCACTTGCACCTTCACCCTCTCCTTCTGTAAGGACAATCAAATAAACTTCTTCTTC
TGTGACCTCCCACCCCTGCTGAAGCTTGCCTGCAGTGACACAGCAAACATCGAGATTGTCATCATCTT
CTTTGGCAATTTTGTGATTTTGGCCAATGCCTCCGTCATCCTGATTTCCTATCTGCTCATCATCAAGA
CCATTTTGAAAGTGAAGTCTTCAGGTGGCAGGGCCAAGACTTTCTCCACATGTGCCTCTCACATCACT
GCTGTGGCCCTTTTCTTTGGAGCCCTTATCTTCATGTATCTGCAAAGTGGCTCAGGCAAATCTCTGGA
GGAAGACAAAGTCGTGTCTGTCTTCTATACAGTGGTCATCCCCATGCTGAACCCTCTGATCTACAGCT
TAAGAAACAAAGATGTAAAAGACGCCTTCAGAAAGGTCGCTAGGAGACTCCAGGTGTTCCTGAGCATG
CTCGAG

NOV11e, CG50315-03

| | | | |
|---|---|---|---|
| Protein Sequence | SEQ ID NO: 132 | 270 aa | MW at 30001.3kD |

MIMLIQVDVKLYTPMYFFLSHLSLLDACYTSVITPQILAThATGKTVISYGHCAAQFFLFTICAGTEC
FLLAVMAYDRYAAIRNPLLYTVAMNPRLYWSLVVGAYVCGVSGAILRTTCTFTLSFCKDNQINFFFCD
LPPLLKLACSDTANIEIVIIFFGNFVILANASVILISYLLIIKTILKVKSSGGRAKTFSTCASHITAV
ALFFGALIFMYLQSGSGKSLEEDKVVSVFYTVVIPMLNPLIYSLRNKDVKDAFRKVARRLQVFLSM

A ClustalW comparison of the above protein sequences yields the following sequence alignment shown in Table 11B.

TABLE 11B
Comparison of the NOV11 protein sequences.

| | |
|---|---|
| NOV11a | --------NLTRVTEFILMGFMDHPKLEIPLFLVFLSFYLVTLLGNVGMIMLIQVDVKYL |
| NOV11b | ----------------------------------------------GSMIMLIQVDVKLY |
| NOV11c | TGSTMAKNNLTRVTEFILMGFMDHPKLEIPLFLVFLSFYLVTLLGNVGMIMLIQVDVKYL |
| NOV11d | ----MAKNNLTRVTEFILMGFMDHPKLEIPLFLVFLSFYLVTLLGNVGMIMLIQVDVKYL |
| NOV11e | ------------------------------------------------MIMLIQVDVKLY |
| NOV11a | TPMYFFLSHLSLLDACYTSVITPQILATLATGKTVISYGHCAAQFFLFTICAGTECFLLA |
| NOV11b | TPMYFFLSHLSLLDACYTSVITPQILATLATGKTVISYGHCAAQFFLFTICAGTECFLLA |
| NOV11c | TPMYFFLSHLSLLDACYTSVITPQILATLATGKTVISYGHCAAQFFLFTICAGTECFLLA |
| NOV11d | TPMYFFLSHLSLLDACYTSVITPQILATLATGKTVISYGHCAAQFFLFTICAGTECFLLA |
| NOV11e | TPMYFFLSHLSLLDACYTSVITPQILATLATGKTVISYGHCAAQFFLFTICAGTECFLLA |
| NOV11a | VMAYDRYAAIRNPLLYTVANNPRLYWSLVVGAYVCGVSGAILRTTCTFTLSFCKDNQINF |
| NOV11b | VMAYDRYAAIRNPLLYTVANNPRLYWSLVVGAYVCGVSGAILRTTCTFTLSFCKDNQINF |
| NOV11c | VMAYDRYAAIRNPLLYTVANNPRLCWSLVVGAYVCGVSGAILRTTCTFTLSFCKDNQINF |
| NOV11d | VMAYDRYAAIRNPLLYTVANNPRLCWSLVVGAYVCGVSGAILRTTCTFTLSFCKDNQINF |
| NOV11e | VMAYDRYAAIRNPLLYTVANNPRLYWSLVVGAYVCGVSGAILRTTCTFTLSFCKDNQINF |
| NOV11a | FFCDLPPLLKLACSDTANIEIVIIFFGNFVILANASVILISYLLIIKTILKVKSSGGRAK |
| NOV11b | FFCDLPPLLKLACSDTANIEIVIIFFGNFVILANASVILISYLLIIKTILKVKSSGGRAK |
| NOV11c | FFCDLPPLLKLACSDTANIEIVIIFFGNFVILANASVILISYLLIIKTILKVKSSGGRAK |
| NOV11d | FFCDLPPLLKLACSDTANIEIVIIFFGNFVILANASVILISYLLIIKTILKVKSSGGRAK |
| NOV11e | FFCDLPPLLKLACSDTANIEIVIIFFGNFVILANASVILISYLLIIKTILKVKSSGGRAK |
| NOV11a | TFSTCASHITAVALFFGALIFMYLQSGSGKSLEEDKVVSVFYTVVIPMLNPLIYSLRNKD |
| NOV11b | TFSTCASHITAVALFFGALIFMYLQSGSGKSLEEDKVVSVFYTVVIPMLNPLIYSLRNKD |
| NOV11c | TFSTCASHITAVALFFGALIFMYLQSGSGKSLEEDKVVSVFYTVVIPMLNPLIYSLRNKD |
| NOV11d | TFSTCASHITAVALFFGALIFMYLQSGSGKSLEEDKVVSVFYTVVIPMLNPLIYSLRNKD |
| NOV11e | TFSTCASHITAVALFFGALIFMYLQSGSGKSLEEDKVVSVFYTVVIPMLNPLIYSLRNKD |
| NOV11a | VKDAFRKVARRLQVSLSM--- |
| NOV11b | VKDAFRKVARRLQVFLSMLE- |
| NOV11c | VKDAFRKVARRLQVSLSMLEG |
| NOV11d | VKDAFRKVARRLQVSLSM--- |
| NOV11e | VKDAFRKVARRLQVFLSM--- |
| NOV11a | (SEQ ID NO: 124) |
| NOV11b | (SEQ ID NO: 126) |
| NOV11c | (SEQ ID NO: 128) |
| NOV11d | (SEQ ID NO: 130) |
| NOV11e | (SEQ ID NO: 132) |

Further analysis of the NOV11a protein yielded the following properties shown in Table 11C.

TABLE 11C
Protein Sequence Properties NOV11a

SignalP analysis: Cleavage site between residues 38 and 39
PSORT II analysis:

| | |
|---|---|
| PSG: | a new signal peptide prediction method |
| | N-region: length 7; pos.chg 1; neg.chg 1 |
| | H-region: length 7; peak value 6.23 |
| | PSG score: 1.83 |
| GvH: | von Heijne's method for signal seq. recognition |
| | GvH score (threshold: −2.1): −3.00 |
| | possible cleavage site: between 37 and 38 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 6 | |
| | INTEGRAL | Likelihood = −6.58 Transmembrane 21-37 |
| | INTEGRAL | Likelihood = −4.09 Transmembrane 97-113 |
| | INTEGRAL | Likelihood = −1.65 Transmembrane 138-154 |
| | INTEGRAL | Likelihood = −8.70 Transmembrane 202-218 |
| | INTEGRAL | Likelihood = −4.14 Transmembrane 240-256 |
| | INTEGRAL | Likelihood = −3.13 Transmembrane 269-285 |
| | PERIPHERAL | Likelihood = 0.85 (at 183) |

| | |
|---|---|
| | ALOM score: −8.70 (number of TMSs: 6) |
| MTOP: | Prediction of membrane topology (Hartmann et al.) |
| | Center position for calculation: 28 |
| | Charge difference: 2.0 C(0.5) − N(−1.5) |
| | C > N: C-terminal side will be inside |

>>>Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75): | 1.96 |
| Hyd Moment(95): | 9.20 | G content: | 1 |
| D/E content: | 2 | S/T content: | 2 |
| Score: −6.22 | | | |

| | |
|---|---|
| Gavel: | prediction of cleavage sites for mitochondrial preseq |
| | R-2 motif at 14 TRV\|TE |
| NUCDISC: | discrimination of nuclear localization signals |
| | pat4: none |
| | pat7: none |
| | bipartite: none |
| | content of basic residues: 7.7% |
| | NLS Score: −0.47 |

KDEL: ER retention motif in the C-terminus: none
ER Membrane Retention Signals:

XXRR-like motif in the N-terminus: LTRV

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG50315-01 is end (k = 9)

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB06632 | G protein-coupled receptor | 1 . . . 310 | 310/310 (100%) | e−176 |
| | GPCR17b protein SEQ ID NO: 74 - | 1 . . . 310 | 310/310 (100%) | |
| | Homo sapiens, 310 aa. | | | |
| | [WO200212343-A2, 14-FEB- | | | |
| | 2002] | | | |
| ABB06631 | G protein-coupled receptor | 1 . . . 310 | 309/310 (99%) | e−175 |
| | GPCR17a protein SEQ ID NO: 72 - | 5 . . . 314 | 309/310 (99%) | |
| | Homo sapiens, 314 aa. | | | |
| | [WO200212343-A2, 14-FEB- | | | |
| | 2002] | | | |
| AAU95529 | Human olfactory and pheromone | 1 . . . 310 | 309/310 (99%) | e−175 |
| | G protein-coupled receptor #16 - | 5 . . . 314 | 309/310 (99%) | |
| | Homo sapiens, 314 aa. | | | |
| | [WO200224726-A2, 28-MAR- | | | |
| | 2002] | | | |
| ABP95742 | Human GPCR polypeptide SEQ | 1 . . . 310 | 309/310 (99%) | e−175 |
| | ID NO 294 - Homo sapiens, 314 | 5 . . . 314 | 309/310 (99%) | |
| | aa. [WO200216548-A2, 28-FEB- | | | |
| | 2002] | | | |
| AAG72468 | Human OR-like polypeptide | 1 . . . 310 | 309/310 (99%) | e−175 |
| | query sequence, SEQ ID NO: | 5 . . . 314 | 309/310 (99%) | |
| | 2149 - Homo sapiens, 314 aa. | | | |
| | [WO200127158-A2, 19-APR- | | | |
| | 2001] | | | |

In a BLAST search of public sequence databases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NGQ6 | Seven transmembrane helix | 1 . . . 310 | 309/310 (99%) | e−175 |
| | receptor - Homo sapiens | 5 . . . 314 | 309/310 (99%) | |
| | (Human), 314 aa. | | | |
| CAD37586 | Sequence 201 from Patent | 1 . . . 310 | 308/310 (99%) | e−173 |
| | WO0224726 - Homo sapiens | 5 . . . 314 | 308/310 (99%) | |
| | (Human), 314 aa. | | | |
| Q8VG66 | Olfactory receptor MOR211-1 - | 1 . . . 307 | 253/307 (82%) | e−146 |
| | Mus musculus (Mouse), 316 aa. | 5 . . . 311 | 279/307 (90%) | |
| Q8VG65 | Olfactory receptor MOR211-2 - | 1 . . . 310 | 246/310 (79%) | e−144 |
| | Mus musculus (Mouse), 314 aa. | 5 . . . 314 | 278/310 (89%) | |
| Q96RA8 | Olfactory receptor - Homo | 64 . . . 279 | 215/216 (99%) | e−119 |
| | sapiens (Human), 216 aa | 1 . . . 216 | 215/216 (99%) | |
| | (fragment). | | | |

PFam analysis indicates that the NOV11a protein contains the domains shown in the Table 11F.

TABLE 11F
Domain Analysis of NOV11a

| | NOV11a | Identities/ | |
|---|---|---|---|
| | Match | Similarities | |
| Pfam Domain | Region | for the Matched Region | Expect Value |
| 7tm_1 | 37 . . . 286 | 55/276 (20%) | 1.5e−28 |
| | | 174/276 (63%) | |

### Example 12

The NOV12 clone was analyzed, and the nucleotide and encoded polypeptide sequences are shown in Table 12A.

TABLE 12A
NOV12 Sequence Analysis

| | | |
|---|---|---|
| NOV12a, CG50341-01 | SEQ ID NO: 133 | 997 bp |

| | | |
|---|---|---|
| DNA Sequence | ORF Start: ATG at 21 | ORF Stop: TAA at 966 |

GGCGCTTATAATTTTGAACTATGACCAATCAGACACAGATGATGGAATTCTTGCTTGTGAGATTTACT
GAGAATTGGGTGCTCCTGAGGCTGCATGCTTTGCTCTTCTCACTGATCTACCTCACGGCTGTGCTGAT
GAATTTAGTCATCATTCTCCTCATGATTCTGGACCATCGTCTCCACATGGCAATGTACTTTTTCCTCC
GACATTTGTCCTTCTTAGACCTGTGTCTCATTTCTGCCACAGTCCCCAAATCCATCCTCAACTCTGTC
GCCTCCACTGACTCCATCTCCTTCCTGGGGTGTGTGTTGCAGCTCTTCTTGGTGGTACTGCTGGCTGG
ATCAGAGATTGGCATCCTTACTGCCATGTCCTATGACCGCTATGCTGCCATCTGCTGCCCCCTACACT
GTGAGGCTGTCATGAGCAGAGGGCTCTGTGTCCAGTTGATGGCTCTGTCCTGGCTCAACAGAGGGGCC
TTGGGACTCTTGTACACAGCTGGAACATTCTCTCTGAATTTTTATGGCTCTGATGAGCTACATCAGTT
CTTCTGCGATGTCCCTGCCCTACTAAAGCTCACTTGTTCTAAAGAACATGCCATCATTAGTGTCAGTG
TGGCCATTGGGGTCTGTTATGCATTTTCATGTTTAGTTTGCATTGTAGTTTCCTATGTGTACATTTTC
TCTGCTGTGTTAAGGATATCACAGAGACAGAGACAATCCAAAGCCTTTTCCAACTGTGTGCCTCACCT
CATTGTTGTCACTGTGTTTCTTGTAACAGGTGCTGTTGCTTATTTAAAGCCAGGGTCTGATGCACCTT
CTATTCTAGACTTGCTGGTGTCTGTGTTCTATTCTGTCGCACCTCCAACCTTGAACCCTGTTATCTAC
TGTCTGAAGAACAAGGACATTAAATCCGCTCTGAGTAAAGTCCTGTGGAATGTTAGAAGCAGTGGGGT
AATGAAAGATGACTAAAGTTGAAGATGGGAAGTACTTTTTTTGNN

NOV12a, CG50341-01

| | | | |
|---|---|---|---|
| Protein Sequence | SEQ ID NO: 134 | 315 aa | MW at 34934.2kD |

MTNQTQMMEFLLVRFTENWVLLRLHALLFSLIYLTAVLMNLVIILLMILDHRLHMAMYFFLRHLSFLD
LCLISATVPKSILNSVASTDSISFLGCVLQLFLVVLLAGSEIGILTAMSYDRYAAICCPLHCEAVMSR
GLCVQLMALSWLNRGALGLLYTAGTFSLNFYGSDELHQFFCDVPALLKLTCSKEHAIISVSVAIGVCY
AFSCLVCIVVSYVYIFSAVLRISQRQRQSKAFSNCVPHLIVVTVFLVTGAVAYLKPGSDAPSILDLLV
SVFYSVAPPTLNPVIYCLKNKDIKSALSKVLWNVRSSGVMKDD

| | | |
|---|---|---|
| NOV12b, 169475616 | SEQ ID NO: 135 | 819 bp |

| | | |
|---|---|---|
| DNA Sequence | ORF Start: at 1 | ORF Stop: end of sequence |

GGATCCATGATTCTGGACCATCGTCTCCACATGGCAATGTACTTTTTCCTCCGACATTTGTCCTTCTT
AGACCTGTGTCTCATTTCTGCCACAGTCCCCAAATCCATCCTCAACTCTGTCGCCTCCACTGACTCCA
TCTCCTTCCTGGGGTGTGTGTTGCAGCTCTTCTTGGTGGTACTGCTGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTCTGAATTTTTATGGCTCTGATGAGCTACATCAGTTCTTCTGCGATGTCCCT
GCCCTACTAAAGCTCACTTGTTCTAAAGAACATGCCATCATTAGTGTCAGTGTGGCCATTGGGGTCTG
TTATGCATTTTCATGTTTAGTTTGCATTGTAGTTTCCTATGTGTACATTTTCTCTGCTGTGTTAAGGA
TATCACAGAGACAGAGACAATCCAAAGCCTTTTCCAACTGTGTGCCTCACCTCATTGTTGTCACTGTG
TTTCTTGTAACAGGTGCTGTTGCTTATTTAAAGCCAGGGTCTGATGCACCTTCTATTCTAGACTTGCT
GGTGTCTGTGTTCTATTCTGTCGCACCTCCAACCTTGAACCCTGTTATCTACTGTCTGAAGAACAAGG
ACATTAAATCCGCTCTGAGTAAAGTCCTGTGGAATGTTAGAAGCAGTGGGGTAATGAAAGATGACCTC
GAG

NOV12b, 169475616

| | | | |
|---|---|---|---|
| Protein Sequence | SEQ ID NO: 136 | 273 aa | MW at 29857.9kD |

GSMILDHRLHMAMYFFLRHLSFLDLCLISATVPKSILNSVASTDSISFLGCVLQLFLVVLLAGSEIGI
LTAMSYDRYAAICCPLHCEAVMSRGLCVQLMALSWLNRGALGLLYTAGTFSLNFYGSDELHQFFCDVP
ALLKLTCSKEHAIISVSVAIGVCYAFSCLVCIVVSYVYIFSAVLRISQRQRQSKAFSNCVPHLIVVTV
FLVTGAVAYLKPGSDAPSILDLLVSVFYSVAPPTLNPVIYCLKNKDIKSALSKVLWNVRSSGVMKDDL
E

| | | |
|---|---|---|
| NOV12c, CG50341-02 | SEQ ID NO: 137 | 996 bp |

| | | |
|---|---|---|
| DNA Sequence | ORF Start: ATG at 21 | ORF Stop: TAA at 966 |

GGCGCTTATAATTTTGAACTATGACCAATCAGACACAGATGATGGAATTCTTGCTTGTGAGATTTACT
GAGAATTGGGTGCTCCTGAGGCTGCATGCTTTGCTCTTCTCACTGATCTACCTCACGGCTGTGCTGAT
GAATTTAGTCATCATTCTCCTCATGATTCTGGACCATCGTCTCCACATGGCAATGTACTTTTTCCTCC
GACATTTGTCCTTCTTAGACCTGTGTCTCATTTCTGCCACAGTCCCCAAATCCATCCTCAACTCTGTC
GCCTCCACTGACTCCATCTCCTTCCTGGGGTGTGTGTTGCAGCTCTTCTTGGTGGTACTGCTGGCTGG
ATCAGAGATTGGCATCCTTACTGCCATGTCCTATGACCGCTATGCTGCCATCTGCTGCCCCCTACACT
GTGAGGCTGTCATGAGCAGAGGGCTCTGTGTCCAGTTGATGGCTCTGTCCTGGCTCAACAGAGGGGCC
TTGGGACTCTTGTACACAGCTGGAACATTCTCTCTGAATTTTTATGGCTCTGATGAGCTACATCAGTT
CTTCTGCGATGTCCCTGCCCTACTAAAGCTCACTTGTTCTAAAGAACATGCCATCATTAGTGTCAGTG
TGGCCATTGGGGTCTGTTATGCATTTTCATGTTTAGTTTGCATTGTAGTTTCCTATGTGTACATTTTC
TCTGCTGTGTTAAGGATATCACAGAGACAGAGACAATCCAAAGCCTTTTCCAACTGTGTGCCTCACCT
CATTGTTGTCACTGTGTTTCTTGTAACAGGTGCTGTTGCTTATTTAAAGCCAGGGTCTGATGCACCTT
CTATTCTAGACTTGCTGGTGTCTGTGTTCTATTCTGTCGCACCTCCAACCTTGAACCCTGTTATCTAC
TGTCTGAAGAACAAGGACATTAAATCCGCTCTGAGTAAAGTCCTGTGGAATGTTAGAAGCAGTGGGGT
AATGAAAGATGACTAAAGATTGAAGATGGGAAGTACTTTTTTTG

NOV12c, CG50341-02

| | | | |
|---|---|---|---|
| Protein Sequence | SEQ ID NO: 138 | 315 aa | MW at 34934.2kD |

MTNQTQMMEFLLVRFTENWVLLRLHALLFSLIYLTAVLMNLVIILLMILDHRLHMAMYFFLRHLSFLD
LCLISATVPKSILNSVASTDSISFLGCVLQLFLVVLLAGSEIGILTAMSYDRYAAICCPLHCEAVMSR
GLCVQLMALSWLNRGALGLLYTAGTFSLNFYGSDELHQFFCDVPALLKLTCSKEHAIISVSVAIGVCY
AFSCLVCIVVSYVYIFSAVLRISQRQRQSKAFSNCVPHLIVVTVFLVTGAVAYLKPGSDAPSILDLLV
SVFYSVAPPTLNPVIYCLKNKDIKSALSKVLWNVRSSGVMKDD

| | | |
|---|---|---|
| NOV12d, CG50341-03 | SEQ ID NO: 139 | 819 bp |

| | | |
|---|---|---|
| DNA Sequence | ORF Start: ATG at 7 | ORF Stop: at 814 |

GGATCCATGATTCTGGACCATCGTCTCCACATGGCAATGTACTTTTTCCTCCGACATTTGTCCTTCTT
AGACCTGTGTCTCATTTCTGCCACAGTCCCCAAATCCATCCTCAACTCTGTCGCCTCCACTGACTCCA
TCTCCTTCCTGGGGTGTGTGTTGCAGCTCTTCTTGGTGGTACTGCTGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTCTGAATTTTTATGGCTCTGATGAGCTACATCAGTTCTTCTGCGATGTCCCT
GCCCTACTAAAGCTCACTTGTTCTAAAGAACATGCCATCATTAGTGTCAGTGTGGCCATTGGGGTCTG
TTATGCATTTTCATGTTTAGTTTGCATTGTAGTTTCCTATGTGTACATTTTCTCTGCTGTGTTAAGGA
TATCACAGAGACAGAGACAATCCAAAGCCTTTTCCAACTGTGTGCCTCACCTCATTGTTGTCACTGTG
TTTCTTGTAACAGGTGCTGTTGCTTATTTAAAGCCAGGGTCTGATGCACCTTCTATTCTAGACTTGCT
GGTGTCTGTGTTCTATTCTGTCGCACCTCCAACCTTGAACCCTGTTATCTACTGTCTGAAGAACAAGG
ACATTAAATCCGCTCTGAGTAAAGTCCTGTGGAATGTTAGAAGCAGTGGGGTAATGAAAGATGACCTC
GAG

NOV12d, CG50341-03

| | | | |
|---|---|---|---|
| Protein Sequence | SEQ ID NO: 140 | 269 aa | MW at 29471.5kD |

MILDHRLHMAMYFFLRHLSFLDLCLISATVPKSILNSVASTDSISFLGCVLQLFLVVLLAGSEIGILT
ANSYDRYAAICCPLHCEAVMSRGLCVQLMALSWLNRGALGLLYTAGTFSLNFYGSDELHQFFCDVPAL
LKLTCSKEHAIISVSVAIGVCYAFSCLVCIVVSYVYIFSAVLRISQRQRQSKAFSNCVPHLIVVTVFL
VTGAVAYLKPGSDAPSILDLLVSVFYSVAPPTLNPVIYCLKNKDIKSALSKVLWNVRSSGVMKDD

| | | |
|---|---|---|
| NOV12e, CG50341-04 | SEQ ID NO: 141 | 957 bp |

| | | |
|---|---|---|
| DNA Sequence | ORF Start: ATG at 7 | ORF Stop: at 952 |

GGATCCATGACCAATCAGACACAGATGATGGAATTCTTGCTTGTGAGATTTACTGAGAATTGGGTGCT
CCTGAGGCTGCATGCTTTGCTCTTCTCACTGATCTACCTCACGGCTGTGCTGATGAATTTAGTCATCA
TTCTCCTCATGATTCTGGACCATCGTCTCCACATGGCAATGTACTTTTTCCTCCGACATTTGTCCTTC
TTAGACCTGTGTCTCATTTCTGCCACAGTCCCCAAATCCATCCTCAACTCTGTCGCCTCCACTGACTC
CATCTCCTTCCTGGGGTGTGTGTTGCAGCTCTTCTTGGTGGTACTGCTGGCTGGATCAGAGATTGGCA
TCCTTACTGCCATGTCCTATGACCGCTATGCTGCCATCTGCTGCCCCCTACACTGTGAGGCTGTCATG
AGCAGAGGGCTCTGTGTCCAGTTGATGGCTCTGTCCTGGCTCAACAGAGGGGCCTTGGGACTCTTGTA
CACAGCTGGAACATTCTCTCTGAATTTTTATGGCTCTGATGAGCTACATCAGTTCTTCTGCGATGTCC
CTGCCCTACTAAAGCTCACTTGTTCTAAAGAACATGCCATCATTAGTGTCAGTGTGGCCATTGGGGTC
TGTTATGCATTTTCATGTTTAGTTTGCATTGTAGTTTCCTATGTGTACATTTTCTCTGCTGTGTTAAG
GATATCACAGAGACAGAGACAATCCAAAGCCTTTTCCAACTGTGTGCCTCACCTCATTGTTGTCACTG
TGTTTCTTGTAACAGGTGCTGTTGCTTATTTAAAGCCAGGGTCTGATGCACCTTCTATTCTAGACTTG
CTGGTGTCTGTGTTCTATTCTGTCGCACCTCCAACCTTGAACCCTGTTATCTACTGTCTGAAGAACAA
GGACATTAAATCCGCTCTGAGTAAAGTCCTGTGGAATGTTAGAAGCAGTGGGGTAATGAAAGATGACC
TCGAG

NOV12e, CG50341-04

| | | | |
|---|---|---|---|
| Protein Sequence | SEQ ID NO: 142 | 315 aa | MW at 34934.2kD |

MTNQTQMMEFLLVRFTENWVLLRLHALLFSLIYLTAVLMNLVIILLMILDHRLHMAMYFFLRHLSFLD
LCLISATVPKSILNSVASTDSISFLGCVLQLFLVVLLAGSEIGILTAMSYDRYAAICCPLHCEAVMSR
GLCVQLMALSWLHRGALGLLYTAGTFSLNFYGSDELHQFFCDVPALLKLTCSKEHAIISVSVAIGVCY
AFSCLVCIVVSYVYIFSAVLRISQRQRQSKAFSNCVPHLIVVTVFLVTGAVAYLKPGSDAPSILDLLV
SVFYSVAPPTLNPVIYCLKNKDIKSALSKVLWNVRSSGVMKDD

A ClustalW comparison of the above protein sequences yields the following sequence alignment shown in Table 12B.

TABLE 12B
Comparison of the NOV12 protein sequences.

| | |
|---|---|
| NOV12a | MTNQTQMMEFLLVRFTENWVLLRLHALLFSLIYLTAVLMNLVIILLMILDHRLHMAMYFF |
| NOV12b | --------------------------------------------GSMILDHRLHMAMYFF |
| NOV12c | MTNQTQMMEFLLVRFTENWVLLRLHALLFSLIYLTAVLMNLVIILLMILDHRLHMAMYFF |
| NOV12d | ----------------------------------------------MILDHRLHMAMYFF |
| NOV12e | MTNQTQMMEFLLVRFTENWVLLRLHALLFSLIYLTAVLMNLVIILLMILDHRLHMAMYFF |
| NOV12a | LRHLSFLDLCLISATVPKSILNSVASTDSISFLGCVLQLFLVVLLAGSEIGILTANSYDR |
| NOV12b | LRHLSFLDLCLISATVPKSILNSVASTDSISFLGCVLQLFLVVLLAGSEIGILTANSYDR |
| NOV12c | LRHLSFLDLCLISATVPKSILNSVASTDSISFLGCVLQLFLVVLLAGSEIGILTANSYDR |
| NOV12d | LRHLSFLDLCLISATVPKSILNSVASTDSISFLGCVLQLFLVVLLAGSEIGILTANSYDR |
| NOV12e | LRHLSFLDLCLISATVPKSILNSVASTDSISFLGCVLQLFLVVLLAGSEIGILTANSYDR |
| NOV12a | YAAICCPLHCEAVMSRGLCVQLMALSWLNRGALGLLYTAGTFSLNFYGSDELHQFFCDVP |
| NOV12b | YAAICCPLHCEAVMSRGLCVQLMALSWLNRGALGLLYTAGTFSLNFYGSDELHQFFCDVP |
| NOV12c | YAAICCPLHCEAVMSRGLCVQLMALSWLNRGALGLLYTAGTFSLNFYGSDELHQFFCDVP |
| NOV12d | YAAICCPLHCEAVMSRGLCVQLMALSWLNRGALGLLYTAGTFSLNFYGSDELHQFFCDVP |
| NOV12e | YAAICCPLHCEAVMSRGLCVQLMALSWLNRGALGLLYTAGTFSLNFYGSDELHQFFCDVP |
| NOV12a | ALLKLTCSKEHAIISVSVAIGVCYAFSCLVCIVVSYVYIFSAVLRISQRQRQSKAFSNCV |
| NOV12b | ALLKLTCSKEHAIISVSVAIGVCYAFSCLVCIVVSYVYIFSAVLRISQRQRQSKAFSNCV |
| NOV12c | ALLKLTCSKEHAIISVSVAIGVCYAFSCLVCIVVSYVYIFSAVLRISQRQRQSKAFSNCV |
| NOV12d | ALLKLTCSKEHAIISVSVAIGVCYAFSCLVCIVVSYVYIFSAVLRISQRQRQSKAFSNCV |
| NOV12e | ALLKLTCSKEHAIISVSVAIGVCYAFSCLVCIVVSYVYIFSAVLRISQRQRQSKAFSNCV |
| NOV12a | PHLIVVTVFLVTGAVAYLKPGSDAPSILDLLVSVFYSVAPPTLNPVIYCLKNKDIKSALS |
| NOV12b | PHLIVVTVFLVTGAVAYLKPGSDAPSILDLLVSVFYSVAPPTLNPVIYCLKNKDIKSALS |
| NOV12c | PHLIVVTVFLVTGAVAYLKPGSDAPSILDLLVSVFYSVAPPTLNPVIYCLKMKDIKSALS |
| NOV12d | PHLIVVTVFLVTGAVAYLKPGSDAPSILDLLVSVFYSVAPPTLNPVIYCLKNKDIKSALS |
| NOV12e | PHLIVVTVFLVTGAVAYLKPGSDAPSILDLLVSVFYSVAPPTLNPVIYCLKNKDIKSALS |
| NOV12a | KVLWNVRSSGVMKDD-- |
| NOV12b | KVLWNVRSSGVMKDDLE |
| NOV12c | KVLWNVRSSGVMKDD-- |
| NOV12d | KVLWNVRSSGVMKDD-- |
| NOV12e | KVLWNVRSSGVMKDD-- |
| NOV12a | (SEQ ID NO: 134) |
| NOV12b | (SEQ ID NO: 136) |
| NOV12c | (SEQ ID NO: 138) |
| NOV12d | (SEQ ID NO: 140) |
| NOV12e | (SEQ ID NO: 142) |

Further analysis of the NOV12a protein yielded the following properties shown in Table 12C.

TABLE 12C
Protein Sequence Properties NOV12a

SignalP analysis: Cleavage site between residues 47 and 48
PSORT II analysis:

| | |
|---|---|
| PSG: | a new signal peptide prediction method |
| N-region: | length 9; pos.chg 0; neg.chg 1 |
| H-region: | length 4; peak value 0.00 |
| PSG score: | −4.40 |
| GvH: | von Heijne's method for signal seq. recognition |
| | GvH score (threshold: −2.1): −3.11 |
| | possible cleavage site: between 36 and 37 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 5 | |
| | INTEGRAL | Likelihood = −9.87 Transmembrane 32-48 |
| | INTEGRAL | Likelihood = −9.66 Transmembrane 90-106 |
| | INTEGRAL | Likelihood = −9.71 Transmembrane 198-214 |
| | INTEGRAL | Likelihood = −5.79 Transmembrane 239-255 |
| | INTEGRAL | Likelihood = 0.16 Transmembrane 267-283 |
| | PERIPHERAL | Likelihood = 1.11 (at 56) |

| | ALOM score: −9.87 (number of TMSs: 5) |
|---|---|
| MTOP: | Prediction of membrane topology (Hartmann et al.) |
| | Center position for calculation: 39 |
| | Charge difference: 1.0 C(2.5) − N(1.5) |
| | C > N: C-terminal side will be inside |

>>> membrane topology: type 3b
MITDISC: discrimination of mitochondrial targeting seq

| | | | |
|---|---|---|---|
| R content: | 1 | Hyd Moment(75): | 2.94 |
| Hyd Moment(95): | 4.71 | G content: | 0 |
| D/E content: | 2 | S/T content: | 3 |
| Score: −5.83 | | | |

| | |
|---|---|
| Gavel: | prediction of cleavage sites for mitochondrial preseq |
| | cleavage site motif not found |
| NUCDISC: | discrimination of nuclear localization signals |
| | pat4: none |
| | pat7: none |
| | bipartite: none |
| | content of basic residues: 6.7% |
| | NLS Score: −0.47 |

KDEL: ER retention motif in the C-terminus: none
ER Membrane Retention Signals:

KKXX-like motif in the C-terminus: VMKD

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LHALLFSLIYLTAVLMNLVIIL at 24

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
11.1%: mitochondrial
11.1%: Golgi
11.1%: vacuolar
11.1%: vesicles of secretory system
11.1%: cytoplasmic

>> prediction for CG50341-01 is end (k = 9)

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU11100 | Human novel G protein-coupled | 1 . . . 315 | 315/315 (100%) | e−179 |
| | receptor, NOV7 - Homo sapiens, | 1 . . . 315 | 315/315 (100%) | |
| | 315 aa. [WO200177177-A2, 18- | | | |
| | OCT-2001] | | | |
| AAU85251 | G-coupled olfactory receptor | 1 . . . 315 | 315/315 (100%) | e−179 |
| | #112 - Homo sapiens, 315 aa. | 1 . . . 315 | 315/315 (100%) | |
| | [WO200198526-A2, 27-DEC- | | | |
| | 2001] | | | |
| AAU95586 | Human olfactory and pheromone | 1 . . . 315 | 315/315 (100%) | e−179 |
| | G protein-coupled receptor #73 - | 1 . . . 315 | 315/315 (100%) | |
| | Homo sapiens, 315 aa. | | | |
| | [WO200224726-A2, 28-MAR- | | | |
| | 2002] | | | |
| AAU24631 | Human olfactory receptor | 1 . . . 315 | 315/315 (100%) | e−179 |
| | AOLFR125 - Homo sapiens, 315 | 1 . . . 315 | 315/315 (100%) | |
| | aa. [WO200168805-A2, 20-SEP- | | | |
| | 2001] | | | |
| ABP95851 | Human GPCR polypeptide SEQ | 1 . . . 313 | 313/313 (100%) | e−178 |
| | ID NO 512 - Homo sapiens, 314 | 1 . . . 313 | 313/313 (100%) | |
| | aa. [WO200216548-A2, 28-FEB- | | | |
| | 2002] | | | |

In a BLAST search of public sequence databases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NGZ2 | Seven transmembrane helix | 1 . . . 315 | 315/315 (100%) | e−179 |
| | receptor - Homo sapiens | 1 . . . 315 | 315/315 (100%) | |
| | (Human), 315 aa. | | | |
| Q96R53 | Olfactory receptor - Homo | 66 . . . 282 | 217/217 (100%) | e−120 |
| | sapiens (Human), 217 aa | 1 . . . 217 | 217/217 (100%) | |
| | (fragment). | | | |
| Q8NHC5 | Seven transmembrane helix | 1 . . . 303 | 158/307 (51%) | 9e−88 |
| | receptor - Homo sapiens | 1 . . . 302 | 225/307 (72%) | |
| | (Human), 309 aa. | | | |
| CAD35484 | Sequence 23 from Patent | 1 . . . 303 | 155/303 (51%) | 4e−87 |
| | WO0208289 - Homo sapiens | 1 . . . 303 | 211/303 (69%) | |
| | (Human), 314 aa. | | | |
| Q8VF67 | Olfactory receptor MOR218-3 - | 1 . . . 302 | 156/302 (51%) | 4e−87 |
| | Mus musculus (Mouse), 315 | 5 . . . 306 | 214/302 (70%) | |
| | aa. | | | |

PFam analysis indicates that the NOV12a protein contains the domains shown in the Table 12F.

TABLE 12F
Domain Analysis of NOV12a

| | NOV12a | Identities/ | |
|---|---|---|---|
| | Match | Similarities | |
| Pfam Domain | Region | for the Matched Region | Expect Value |
| 7tm_1 | 39 . . . 288 | 57/277 (21%) | 1.8e−14 |
| | | 170/277 (61%) | |

### Example 13

The NOV13 clone was analyzed, and the nucleotide and encoded polypeptide sequences are shown in Table 13A.

TABLE 13A
NOV13 Sequence Analysis

| | | |
|---|---|---|
| NOV13a, CG50365-01 | SEQ ID NO: 143 | 828 bp |

| | | |
|---|---|---|
| DNA Sequence | ORF Start: ATG at 16 | ORF Stop: TAA at 802 |

CCACCCCGAGGGACCATGTCGAGGCTCAGCTGGGGATACCGCGAGCACAACGGTCCTATTCACTGGAA
GGAATTTTTCCCTATTGCTGATGGTGATCAGCAATCTCCAATTGAGATTAAAACCAAAGAAGTGAAAT
ATGACTCTTCCCTCCGACCACTTAGTATCAAGTATGACCCAAGCTCAGCTAAAATCATCAGCAACAGC
GGCCATTCCTTCAATGTTGACTTTGATGACACAGAGAACAAATCAGTTCTGCGTGGTGGTCCTCTCAC
TGGAAGCTACAGGTTACGGCAGGTTCACCTTCACTGGGGGTCCGCTGATGACCACGGCTCCGAGCACA
TAGTAGATGGAGTGAGCTATGCTGCAGAGCTCCATGTTGTTCACTGGAATTCAGACAAATACCCCAGC
TTTGTTGAGGCAGCTCATGAACCAGATGGACTGGCTGTCTTGGGAGTGTTTTTACAGGTGGGTGAACC
TAATTCCCAACTGCAAAAGATTACTGACACTTTGGATTCCATTAAAGAAAAGGGTAAACAAACTCGAT
TCACAAATTTTGACCTATTGTCTCTGCTTCCACCATCCTGGGACTACTGGACATATCCTGGTTCTCTT
ACAGTTCCACCTCTTCTTGAGAGTGTCACATGGATTGTTTTAAAGCAACCTATAAACATCAGCTCTCA
ACAGCTGGCCAAATTTCGCAGTCTCCTGTGCACAGCGGAGGGTGAAGCAGCAGCTTTTCTGGTGAGCA
ATCACCGCCCACCACAGCCTCTAAAGGGCCGCAAAGTGAGAGCCTCTTTCCATTAAAAATTGTCACCA
ATGAACTCCCCC

NOV13a, CG50365-01

| | | | |
|---|---|---|---|
| Protein Sequence | SEQ ID NO: 144 | 262 aa | MW at 29428.8kD |

MSRLSWGYREHNGPIHWKEFFPIADGDQQSPIEIKTKEVKYDSSLRPLSIKYDPSSAKIISNSGHSFN
VDFDDTENKSVLRGGPLTGSYRLRQVHLHWGSADDHGSEHIVDGVSYAAELHVVHWNSDKYPSFVEAA
HEPDGLAVLGVFLQVGEPNSQLQKITDTLDSIKEKGKQTRFTNFDLLSLLPPSWDYWTYPGSLTVPPL
LESVTWIVLKQPINISSQQLAKFRSLLCTAEGEAAAFLVSNHRPPQPLKGRKVRASFH

| | | |
|---|---|---|
| NOV13b, 278019595 | SEQ ID NO: 145 | 784 bp |

| | | |
|---|---|---|
| DNA Sequence | ORF Start: at 2 | ORF Stop: end of sequence |

CACCGGATCCAGCTGGGGATACCGCGAGCACAACGGTCCTATTCACTGGAAGGAATTTTTCCCTATTG
CTGATGGTGATCAGCAATCTCCAATTGAGATTAAAACCAAAGAAGTGAAATATGACTCTTCCCTCCGA
CCACTTAGTATCAAGTATGACCCAAGCTCAGCTAAAATCATCAGCAACAGCGGCCATTCCTTCAATGT
TGACTTTGATGACACAGAGAACAAATCAGTTCTGCGTGGTGGTCCTCTCACTGGAAGCTACAGGTTAC
GGCAGGTTCACCTTCACTGGGGGTCCGCTGATGACCACGGCTCCGAGCACATAGTAGATGGAGTGAGC
TATGCTGCAGAGCTCCATGTTGTTCACTGGAATTCAGACAAATACCCCAGCTTTGTTGAGGCAGCTCA
TGAACCAGATGGACTGGCTGTCTTGGGAGTGTTTTTACAGATTGGTGAACCTAATTCCCAACTGCAAA
AGATTACTGACACTTTGGATTCCATTAAAGAAAAGGGTAAACAAACTCGATTCACAAATTTTGACCTA
TTGTCTCTGCTTCCACCATCCTGGGACTACTGGACATATCCTGGTTCTCTTACAGTTCCACCTCTTCT
TGAGAGTGTCACATGGATTGTTTTAAAGCAACCTATAAACATCAGCTCTCAACAGCTGGCCAAATTTC
GCAGTCTCCTGTGCACAGCGGAGGGTGAAGCAGCAGCTTTTCTGGTGAGCAATCACCGCCCACCACAG
CCTCTAAAGGGCCGCAAAGTGAGAGCCCTCGAGGGC

NOV13b, 278019595

| | | | |
|---|---|---|---|
| Protein Sequence | SEQ ID NO: 146 | 261 aa | MW at 29128.4kD |

TGSSWGYREHNGPIHWKEFFPIADGDQQSPIEIKTKEVKYDSSLRPLSIKYDPSSAKIISNSGHSFNV
DFDDTEMKSVLRGGPLTGSYRLRQVHLHWGSADDHGSEHIVDGVSYAAELHVVHWNSDKYPSFVEAAH
EPDGLAVLGVFLQIGEPNSOLQKITDTLDSIKEKGKQTRFTNFDLLSLLPPSWDYWTYPGSLTVPPLL
ESVTWIVLKQPINISSQQLAKFRSLLCTAEGEAAAFLVSNHRPPQPLKGRKVRALEG

| | | |
|---|---|---|
| NOV13c, CG50365-02 | SEQ ID NO: 147 | 833 bp |

| | | |
|---|---|---|
| DNA Sequence | ORF Start: ATG at 21 | ORF Stop: TAA at 807 |

ATGTCGAGGCTCAGCTGGGGATGTCGAGGCTCAGCTGGGGATACCGCGAGCACAACGGTCCTATTCAC
TGGAAGGAATTTTTCCCTATTGCTGATGGTGATCAGCAATCTCCAATTGAGATTAAAACCAAAGAAGT
GAAATATGACTCTTCCCTCCGACCACTTAGTATCAAGTATGACCCAAGCTCAGCTAAAATCATCAGCA
ACAGCGGCCATTCCTTCAATGTTGACTTTGATGACACAGAGAACAAATCAGTTCTGCGTGGTGGTCCT
CTCACTGGAAGCTACAGGTTACGGCAGGTTCACCTTCACTGGGGGTCCGCTGATGACCACGGCTCCGA
GCACATAGTAGATGGAGTGAGCTATGCTGCAGAGCTCCATGTTGTTCACTGGAATTCAGACAAATACC
CCAGCTTTGTTGAGGCAGCTCATGAACCAGATGGACTGGCTGTCTTGGGAGTGTTTTTACAGATTGGT
GAACCTAATTCCCAACTGCAAAAGATTACTGACACTTTGGATTCCATTAAAGAAAAGGGTAAACAAAC
TCGATTCACAAATTTTGACCTATTGTCTCTGCTTCCACCATCCTGGGACTACTGGACATATCCTGGTT
CTCTTACAGTTCCACCTCTTCTTGAGAGTGTCACATGGATTGTTTTAAAGCAACCTATAAACATCAGC
TCTCAACAGCTGGCCAAATTTCGCAGTCTCCTGTGCACAGCGGAGGGTGAAGCAGCAGCTTTTCTGGT
GAGCAATCACCGCCCACCACAGCCTCTAAAGGGCCGCAAAGTGAGAGCCTCTTTCCATTAAAAATTGT
CACCAATGAACTCCCCC

NOV13c, CG50365-02

| | | | |
|---|---|---|---|
| Protein Sequence | SEQ ID NO: 148 | 262 aa | MW at 29442.8kD |

MSRLSWGYREHNGPIHWKEFFPIADGDQQSPIEIKTKEVKYDSSLRPLSIKYDPSSAKIISNSGHSFN
VDFDDTENKSVLRGGPLTGSYRLRQVHLHWGSADDHGSEHIVDGVSYAAELHVVHWNSDKYPSFVEAA
HEPDGLAVLGVFLQIGEPNSQLQKITDTLDSIKEKGKQTRFTNFDLLSLLPPSWDYWTYPGSLTVPPL
LESVTWIVLKQPINISSQQLAKFRSLLCTAEGEAAAFLVSNHRPPQPLKGRKVRASFH

A ClustalW comparison of the above protein sequences yields the following sequence alignment shown in Table 13B.

TABLE 13B
Comparison of the NOV13 protein sequences.

| | |
|---|---|
| NOV13a | MSRLSWGYREHNGPIHWKEFFPIADGDQQSPIEIKTKEVKYDSSLRPLSIKYDPSSAKII |
| NOV13b | -TGSSWGYREHNGPIHWKEFFPIADGDQQSPIEIKTKEVKYDSSLRPLSIKYDPSSAKII |
| NOV13c | MSRLSWGYREHNGPIHWKEFFPIADGDQQSPIEIKTKEVKYDSSLRPLSIKYDPSSAKII |
| NOV13a | SNSGHSFNVDFDDTENKSVLRGGPLTGSYRLRQVHLHWGSADDHGSEHIVDGVSYAAELH |
| NOV13b | SNSGHSFNVDFDDTENKSVLRGGPLTGSYRLRQVHLHWGSADDHGSEHIVDGVSYAAELH |
| NOV13c | SNSGHSFNVDFDDTENKSVLRGGPLTGSYRLRQVHLHwGSADDHGSEHIVDGVSYAAELH |
| NOV13a | VVHWNSDKYPSFVEAAHEPDGLAVLGVFLQVGEPNSQLQKITDTLDSIKEKGKQTRFTNF |
| NOV13b | VVHWNSDKYPSFVEAAHEPDGLAVLGVFLQIGEPNSQLQKITDTLDSIKEKGKQTRFTNF |
| NOV13c | VVHWNSDKYPSFVEAAHEPDGLAVLGVFLQIGEPNSQLQKITDTLDSIKEKGKQTRFTNF |
| NOV13a | DLLSLLPPSWDYWTYPGSLTVPPLLESVTWIVLKQPINISSQQLAKFRSLLCTAEGEAAA |
| NOV13b | DLLSLLPPSWDYWTYPGSLTVPPLLESVTWIVLKQPINISSQQLAKFRSLLCTAEGEAAA |
| NOV13c | DLLSLLPPSWDYWTYPGSLTVPPLLESVTWIVLKQPINISSQQLAKFRSLLCTAEGEAAA |
| NOV13a | FLVSNHRPPQPLKGRKVRASFH |
| NOV13b | FLVSNHRPPQPLKGRKVRALEG |
| NOV13c | FLVSNHRPPQPLKGRKVRASFH |
| NOV13a | (SEQ ID NO: 144) |
| NOV13b | (SEQ ID NO: 146) |
| NOV13c | (SEQ ID NO: 148) |

Further analysis of the NOV13a protein yielded the following properties shown in Table 13C.

TABLE 13C
Protein Sequence Properties NOV13a

SignalP analysis: No Known Signal Sequence Predicted
PSORT II analysis:

| | |
|---|---|
| PSG: | a new signal peptide prediction method |
| | N-region: length 10; pos.chg 2; neg.chg 1 |
| | H-region: length 7; peak value −7.23 |
| | PSG score: −11.62 |
| GvH: | von Heijne's method for signal seq. recognition |
| | GvH score (threshold: −2.1): −8.81 |
| | possible cleavage site: between 57 and 58 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 2.97 (at 197) |

ALOM score: 2.97 (number of TMSs: 0)

MITDISC: discrimination of mitochondrial targeting seq

| | | | |
|---|---|---|---|
| R content: | 2 | Hyd Moment(75): | 8.62 |
| Hyd Moment(95): | 7.75 | G content: | 2 |
| D/E content: | 2 | S/T content: | 2 |
| Score: −5.03 | | | |

| | |
|---|---|
| Gavel: | prediction of cleavage sites for mitochondrial preseq |
| | R-2 motif at 13 SRL\|SW |
| NUCDISC: | discrimination of nuclear localization signals |
| | pat4: none |
| | pat7: PLKGRKV (4) at 251 |
| | bipartite: none |
| | content of basic residues: 10.3% |
| | NLS Score: −0.13 |

KDEL: ER retention motif in the C-terminus: none
ER Membrane Retention Signals:

XXRR-like motif in the N-terminus: SRLS

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| | total: 0 residues |

Final Results (k = 9/23):

43.5%: cytoplasmic
34.8%: mitochondrial
21.7%: nuclear

>> prediction for CG50365-01 is cyt (k = 23)

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE25377 | Human NZMS-1 protein - Homo | 1 . . . 262 | 246/262 (93%) | e−143 |
| | sapiens, 247 aa. [WO200246385- | 1 . . . 247 | 247/262 (93%) | |
| | A2, 13-JUN-2002] | | | |
| AAU19418 | Human diagnostic and therapeutic | 1 . . . 262 | 246/262 (93%) | e−143 |
| | polypeptide (DITHP) #4 - Homo | 13 . . . 273 | 248/262 (93%) | |
| | sapiens, 274 aa. [WO200162927- | | | |
| | A2, 30-AUG-2001] | | | |
| ABB08900 | Human lyase HLYA-1 protein - | 1 . . . 242 | 241/242 (99%) | e−143 |
| | Homo sapiens, 242 aa. | 1 . . . 242 | 242/242 (99%) | |
| | [WO200200840-A2, 03-JAN- | | | |
| | 2002] | | | |
| AAB63110 | Human secreted protein sequence | 78 . . . 261 | 181/184 (98%) | e−105 |
| | encoded by gene 27 SEQ ID | 1 . . . 184 | 183/184 (99%) | |
| | NO: 120 - Homo sapiens, 184 aa. | | | |
| | [WO200061748-A1, 19-OCT- | | | |
| | 2000] | | | |
| AAO15236 | Human carbonic anhydrase I | 1 . . . 261 | 157/261 (60%) | 5e−94 |
| | (Cln115) protein - Homo sapiens, | 1 . . . 261 | 202/261 (77%) | |
| | 261 aa. [US2002042088-A1, 11- | | | |
| | APR-2002] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8N1Q1 | Carbonic anhydrase XIII (EC | 1 . . . 262 | 261/262 (99%) | e−155 |
| | 4.2.1.1) (Carbonate dehydratase | 1 . . . 262 | 262/262 (99%) | |
| | XIII) (CA-XIII) - Homo sapiens | | | |
| | (Human), 262 aa. | | | |
| Q9D6N1 | Carbonic anhydrase XIII (EC | 1 . . . 262 | 238/262 (90%) | e−141 |
| | 4.2.1.1) (Carbonate dehydratase | 1 . . . 262 | 250/262 (94%) | |
| | XIII) (CA-XIII) - Mus musculus | | | |
| | (Mouse), 262 aa. | | | |
| Q8HY33 | Carbonic anhydrase 1 - | 1 . . . 261 | 164/261 (62%) | e−99 |
| | Monodelphis domestica (Short- | 1 . . . 261 | 210/261 (79%) | |
| | tailed grey opossum), 262 aa. | | | |
| Q8JG56 | Erythrocyte carbonic anhydrase - | 5 . . . 261 | 165/257 (64%) | 1e−95 |
| | Lepisosteus osseus (Long-nosed | 4 . . . 260 | 198/257 (76%) | |
| | gar), 261 aa. | | | |
| Q8UWA5 | Carbonic anhydrase 2 (EC 4.2.1.1) | 6 . . . 261 | 164/256 (64%) | 8e−95 |
| | (Carbonate dehydratase) - | 5 . . . 259 | 200/256 (78%) | |
| | Tribolodon hakonensis, 260 aa. | | | |

PFam analysis indicates that the NOV13a protein contains the domains shown in the Table 13F.

TABLE 13F
Domain Analysis of NOV13a

| | NOV13a | Identities/ | |
|---|---|---|---|
| | Match | Similarities | |
| Pfam Domain | Region | for the Matched Region | Expect Value |
| carb_anhydrase | 6 . . . 261 | 172/283 (61%) | 1.9e−162 |
| | | 221/283 (78%) | |

### Example 14

The NOV14 clone was analyzed, and the nucleotide and encoded polypeptide sequences are shown in Table 14A.

TABLE 14A
NOV14 Sequence Analysis

| | | |
|---|---|---|
| NOV14a, CG50367-01 | SEQ ID NO: 149 | 2762 bp |

| | | |
|---|---|---|
| DNA Sequence | ORF Start: ATG at 3 | ORF Stop: TGA at 2745 |

CTATGGGCTGGAGGCCCCGGAGAGCTCGGGGGACCCCGTTGCTGCTGCTGCTACTACTGCTGCTGCTC
TGGCCAGTGCCAGGCGCCGGGGTGCTTCAAGGACATATCCCTGGGCAGCCAGTCACCCCGCACTGGGT
CCTGGATGGACAACCCTGGCGCACCGTCAGCCTGGAGGAGCCGGTCTCGAAGCCAGACATGGGGCTGG
TGGCCCTGGAGGCTGAAGGCCAGGAGCTCCTGCTTGAGCTGGAGAAGAACCACAGGCTGCTGGCCCCA
GGATACATAGAAACCCACTACGGCCCAGATGGGCAGCCAGTGGTGCTGGCCCCCAACCACACGGATCA
TTGCCACTACCAAGGGCGAGTAAGGGGCTTCCCCGACTCCTGGGTAGTCCTCTGCACCTGCTCTGGGA
TGAGTGGCCTGATCACCCTCAGCAGGAATGCCAGCTATTATCTGCGTCCCTGGCCACCCCGGGGCTCC
AAGGACTTCTCAACCCACGAGATCTTTCGGATGGAGCAGCTGCTCACCTGGAAAGGAACCTGTGGCCA
CAGGGATCCTGGGAACAAAGCGGGCATGACCAGCCTTCCTGGTGGTCCCCAGAGCAGGGGCAGGCGAG
AAGCGCGCAGGACCCGGAAGTACCTGGAACTGTACATTGTGGCAGACCACACCCTGTTCTTGACTCGG
CACCGAAACTTGAACCACACCAAACAGCGTCTCCTGGAAGTCGCCAACTACGTGGACCAGCTTCTCAG
GACTCTGGACATTCAGGTGGCGCTGACCGGCCTGGAGGTGTGGACCGAGCGGGACCGCAGCCGCGTCA
CGCAGGACGCCAACGCCACGCTCTGGGCCTTCCTGCAGTGGCGCCGGGGGCTGTGGGCGCAGCGGCCC
CACGACTCCGCGCAGCTGCTCACGGGCCGCGCCTTCCAGGGCGCCACAGTGGGCCTGGCGCCCGTCGA
GGGCATGTGCCGCGCCGAGAGCTCGGGAGGCGTGAGCACGGACCACTCGGAGCTCCCCATCGGCGCCG
CAGCCACCATGGCCCATGAGATCGGCCACAGCCTCGGCCTCAGCCACGACCCCGACGGCTGCTGCGTG
GAGGCTGCGGCCGAGTCCGGAGGCTGCGTCATGGCTGCGGCCACCGGGCACCCGTTTCCGCGCGTGTT
CAGCGCCTGCAGCCGCCGCCAGCTGCGCGCCTTCTTCCGCAAGGGGGGCGGCGCTTGCCTCTCCAATG
CCCCGGACCCCGGACTCCCGGTGCCGCCGGCGCTCTGCGGGAACGGCTTCGTGGAAGCGGGCGAGGAG
TGTGACTGCGGCCCTGGCCAGGAGTGCCGCGACCTCTGCTGCTTTGCTCACAACTGCTCGCTGCGCCC
GGGGGCCCAGTGCGCCCACGGGGACTGCTGCGTGCGCTGCCTGCTGAAGCCGGCTGGAGCGCTGTGCC
GCCAGGCCATGGGTGACTGTGACCTCCCTGAGTTTTGCACGGGCACCTCCTCCCACTGTCCCCCAGAC
GTTTACCTACTGGACGGCTCACCCTGTGCCAGGGGCAGTGGCTACTGCTGGGATGGCGCATGTCCCAC
GCTGGAGCAGCAGTGCCAGCAGCTCTGGGGGCCTGGCTCCCACCCAGCTCCCGAGGCCTGTTTCCAGG
TGGTGAACTCTGCGGGAGATGCTCATGGAAACTGCGGCCAGGACAGCGAGGGCCACTTCCTGCCCTGT
GCAGGGAGGGATGCCCTGTGTGGGAAGCTGCAGTGCCAGGGTGGAAAGCCCAGCCTGCTCGCACCGCA
CATGGTGCCAGTGGACTCTACCGTTCACCTAGATGGCCAGGAAGTGACTTGTCGGGGAGCCTTGGCAC
TCCCCAGTGCCCAGCTGGACCTGCTTGGCCTGGGCCTGGTAGAGCCAGGCACCCAGTGTGGACCTAGA
ATGGTGTGCCAGAGCAGGCGCTGCAGGAAGAATGCCTTCCAGGAGCTTCAGCGCTGCCTGACTGCCTG
CCACAGCCACGGGGTTTGCAATAGCAACCATAACTGCCACTGTGCTCCAGGCTGGGCTCCACCCITCT
GTGACAAGCCAGGCTTTGGTGGCAGCATGGACAGTGGCCCTGTGCAGGCTGAAAACCATGACACCTTC
CTGCTGGCCATGCTCCTCAGCGTCCTGCTGCCTCTGCTCCCAGGGGCCGGCCTGGCCTGGTGTTGCTA
CCGACTCCCAGGAGCCCATCTGCAGCGATGCAGCTGGGGCTGCAGAAGGGACCCTGCGTGCAGTGGCC
CCAAAGATGGCCCACACAGGGACCACCCCCTGGGCGGCGITCACCCCATGGAGTTGGGCCCCACAGCC
ACTGGACAGCCCTGGCCCCTGGCCCCAGGGGCTCCTGCTGACCATATTCACAACATTTACCCTCCACC
ATTTCTCCCAGACCCTGAGAACTCTCATGAGCCCAGCAGCCACCCTGAGAAGCCTCTGCCAGCAGTCT
CGCCTGACCCCCAAGGTGGTTCCCTTGCAGCCTGGGGCCCCAGTCCTTTAGGGGACAACATATCCTCC
TCATTCTCAGCAGATCAAGTCCAGATGCCAAGATCCTGCCTCTGTGGCGAACCCTGGGGAGGCCACGT
GGGAAGGAAAGAGGGCTCTAAGAGGGGAGGCCCCAGACTGGGGGAGAGGCCTGTCTGGAGCCCAGGAT
CACCTGGCTGTGCTGCAGAACTGGAGAAGAGAAGCTCAGCAGAAAGGAGCTGGCATGGGGCCAACAGC
AGAAAAGCAGGAGGCACGCAGAAGTGACTGGGAAGCAGGAGG

NOV14a, CG50367-01

| | | | |
|---|---|---|---|
| Protein Sequence | SEQ ID NO: 150 | 914 aa | MW at 98055.2kD |

MGWRPRPARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRMEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLLKPAGALCR
QAMGDCDLPEFCTGTSSHCPPDVYLLDGSPCARGSGYCWDGACPTLEQQCQQLWGPGSHPAPEACFQV
VNSAGDAHGNCGQDSEGHFLPCAGRDALCGKLQCQGGKPSLLAPHMVPVDSTVHLDGQEVTCRGALAL
PSAQLDLLGLGLVEPGTQCGPRMVCQSRRCRKNAFQELQRCLTACHSHGVCNSNHNCHCAPGWAPPFC
DKPGFGGSMDSGPVQAENHDTFLLAMLLSVLLPLLPGAGLAWCCYRLPGAHLQRCSWGCRRDPACSGP
KDGPHRDHPLGGVHPMELGPTATGQPWPLAPGAPADHIHNIYPPPFLPDPENSHEPSSHPEKPLPAVS
PDPQGGSLAAWGPSPLGDNISSSFSADQVQMPRSCLCGEPWGGHVGRKEGSKRGGPRLGERPVWSPGS
PGCAAELEKRSSAERSWHGANSRKAGGTQK

| | | |
|---|---|---|
| NOV14b, CG50367-05 | SEQ ID NO: 151 | 3397 bp |

| | | |
|---|---|---|
| DNA Sequence | ORF Start: ATG at 37 | ORF Stop: TGA at 2398 |

GCGAGCCGCTGCCTAGAGGCCGAGGAGCTCACAGCTATGGGCTGGAGGCCCCGGAGAGCTCGGGGGAC
CCCGTTGCTGCTGCTGCTACTACTGCTGCTGCTCTGGCCAGTGCCAGTCGCCGGGGTGCTTCAAGGAC
ATATCCCTGGGCAGCCAGTCACCCCGCACTGGGTCCTGGATGGACAACCCTGGCGCACCGTCAGCCTG
GAGGAGCCGGTCTCGAAGCCAGACATGGGGCTGGTGGCCCTGGAGGCTGAAGGCCAGGAGCTCCTGCT
TGAGCTGGAGAAGAACCACAGGCTGCTGGCCCCAGGATACATAGAAACCCACTACGGCCCAGATGGGC
AGCCAGTGGTGCTGGCCCCCAACCACACGGATCATTGCCACTACCAAGGGCGAGTAAGGGGCTTCCCC
GACTCCTGGGTAGTCCTCTGCACCTGCTCTGGGATGAGTGGCCTGATCACCCTCAGCAGGAATGCCAG
CTATTATCTGCGTCCCTGGCCACCCCGGGGCTCCAAGGACTTCTCAACCCACGAGATCTTTCGGATGG
AGCAGCTGCTCACCTGGAAAGGAACCTGTGGCCACAGGGATCCTGGGAACAAAGCGGGCATGACCAGC
CTTCCTGGTGGTCCCCAGAGCAGGGGCAGGCGAGAAGCGCGCAGGACCCGGAAGTACCTGGAACTGTA
CATTGTGGCAGACCACACCCTGTTCTTGACTCGGCACCGAAACTTGAACCACACCAAACAGCGTCTCC
TGGAAGTCGCCAACTACGTGGACCAGCTTCTCAGGACTCTGGACATTCAGGTGGCGCTGACCGGCCTG
GAGGTGTGGACCGAGCGGGACCGCAGCCGCGTCACGCAGGACGCCAACGCCACGCTCTGGGCCTTCCT
GCAGTGGCGCCGGGGGCTGTGGGCGCAGCGGCCCCACGACTCCGCGCAGCTGCTCACGGGCCGCGCCT
TCCAGGGCGCCACAGTGGGCCTGGCGCCCGTCGAGGGCATGTGCCGCGCCGAGAGCTCGGGAGGCGTG
AGCACGGACCACTCGGAGCTCCCCATCGGCGCCGCAGCCACCATGGCCCATGAGATCGGCCACAGCCT
CGGCCTCAGCCACGACCCCGACGGCTGCTGCGTGGAGGCTGCGGCCGAGTCCGGAGGCTGCGTCATGG
CTGCGGCCACCGGGCACCCGTTTCCGCGCGTGTTCAGCGCCTGCAGCCGCCGCCAGCTGCGCGCCTTC
TTCCGCAAGGGGGGCGGCGCTTGCCTCTCCAATGCCCCGGACCCCGGACTCCCGGTGCCGCCGGCGCT
CTGCGGGAACGGCTTCGTGGAAGCGGGCGAGGAGTGTGACTGCGGCCCTGGCCAGGAGTGCCGCGACC
TCTGCTGCTTTGCTCACAACTGCTCGCTGCGCCCGGGGGCCCAGTGCGCCCACGGGGACTGCTGCGTG
CGCTGCCTGCTGAAGCCGGCTGGAGCGCTGTGCCGCCAGGCCATGGGTGACTGTGACCTCCCTGAGTT
TTGCACGGGCACCTCCTCCCACTGTCCCCCAGACGTTTACCTACTGGACGGCTCACCCTGTGCCAGGG
GCAGTGGCTACTGCTGGGATGGCGCATGTCCCACGCTGGAGCAGCAGTGCCAGCAGCTCTGGAAGCCT
GGCTCCCACCCAGCTCCCGAGGCCTGTTTCCAGGTGGTGAACTCTGCGGGAGATGCTCATGGAAACTG
CGGCCAGGACAGCGAGGGCCACTTCCTGCCCTGTGCAGGGAGGGATGCCCTGTGTGGGAAGCTGCAGT
GCCAGGGTGGAAAGCCCAGCCTGCTCGCACCGCACATGGTGCCAGTGGACTCTACCGTTCACCTAGAT
GGCCAGGAAGTGACTTGTCGGGGAGCCTTGGACTCCCCAGTGCCCAGCTGGACCTGCTTGGCCTTGGG
CCTGGTAGAGTCAGGCACCCAGTGTGGACCTAGAATGGTTTGCAATAGCAACCATAACTGCCACTGTG
CTCCAGGCTGGGCTCCACCCTTCTGTGACAAGCCAGGCTTTGGTGGCAGCATGGACAGTGGCCCTGTG
CAGGCTGAAAACCATGACACCTTCCTGCTGGCCATGCTCCTCAGCGTCCTGCTGCCTCTGCTCCCAGG
GGCCGGCCTGGCCTGGTGTTGCTACCGACTCCCAGGAGCCCATCTGCAGCGATGCAGCTGGGGCTGCA
GAAGGGACCCTGCGTGCAGTGGCCCCAAAGATGGCCCACACAGGGACCACCCCCTGGGCGGCGTTCAC
CCCATGGAGTTGGGCCCCACAGCCACTGGACAGCCCTGGCCCCTGGACCCTGAGAACTCTCATGAGCC
CAGCAGCCACCCTGAGAAGCCTCTGCCAGCAGTCTCGCCCGACCCCCAAGCAGATCAAGTCCAGATGC
CAAGATCCTGCCTCTGGTGAGAGGTAGCTCCTAAAATGAACAGATTTAAAGACAGGTGGCCACTGACA
GCCACTCCAGGAACTTGAACTGCAGGGGCAGAGCCAGTGAATCACCGGACCTCCAGCACCTGCAGGCA
GCTTGGAAGTTTCTTCCCCGAGTGGAGCTTCGACCCACCCACTCCAGGAACCCAGAGCCACACTAGAA
GTTCCTGAGGGCTGGAGAACACTGCTGGGCACACTCTCCAGCTCAATAAACCATCAGTCCCAGAAGCA
AAGGTCACACAGCCCCTGACCTCCCTCACCAGTGGAGGCTGGGTAGTGCTGGCCATCCCAAAAGGGCT
CTGTCCTGGGAGTCTGGTGTGTCTCCTACATGCAATTTCCACGGACCCAGCTCTGTGGAGGGCATGAC
TGCTGGCCAGAAGCTAGTGGTCCTGGGGCCCTATGGTTCGACTGAGTCCACACTCCCCTGCAGCCTGG
CTGGCCTCTGCAAACAAACATAATTTTGGGGACCTTCCTTCCTGTTTCTTCCCACCCTGTCTTCTCCC
CTAGGTGGTTCCTGAGCCCCCACCCCCAATCCCAGTGCTACACCTGAGGTTCTGGAGCTCAGAATCTG
ACAGCCTCTCCCCCATTCTGTGTGTGTCGGGGGGACAGAGGGAACCATTTAAGAAAAGATACCAAAGT
AGAAGTCAAAAGAAAGACATGTTGGCTATAGGCGTGGTGGCTCATGCCTATAATCCCAGCACTTTGGG
AAGCCGGGGTAGGAGGATCACCAGAGGCCAGCAGGTCCACACCAGCCTGGGCAACACAGCAAGACACC
GCATCTACAGAAAAATTTTAAAATTAGCTGGGCGTGGTGGTGTGTACCTGTAGGCCTAGCTGCTCAGG
AGGCTGAAGCAGGAGGATCACTTGAGCCTGAGTTCAACACTGCAGTGAGCTATGGTGGCACCACTGCA
CTCCAGCCTGGGTGACAGAGCAAGACCCTGTCTCTAAAATAAATTTTAAAAAGACATATTACACT

NOV14b, CG50367-05

| | | | |
|---|---|---|---|
| Protein Sequence | SEQ ID NO: 152 | 787 aa | MW at 84713.5kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRMEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLLKPAGALCR
QAMGDCDLPEFCTGTSSHCPPDVYLLDGSPCARGSGYCWDGACPTLEQQCQQLWGPGSHPAPEACFQV
VNSAGDAHGNCGQDSEGHFLPCAGRDALCGKLQCQGGKPSLLAPHMVPVDSTVNLDGQEVTCRGALAL
PSAQLDLLGLGLVESGTQCGPRNVCNSNHNCHCAPGWAPPFCDKPGFGGSMDSGPVQAENHDTFLLAM
LLSVLLPLLPGAGLAWCCYRLPGAHLQRCSWGCRRDPACSGPKDGPHRDHPLGGVHPMELGPTATGQP
WPLDPENSHEPSSHPEKPLPAVSPDPQADQVQMPRSCLW

| | | |
|---|---|---|
| NOV14c, CG50367-06 | SEQ ID NO: 153 | 3351 bp |

| | | |
|---|---|---|
| DNA Sequence | ORF Start: ATG at 37 | ORF Stop: TGA at 1579 |

GCGAGCCGCTGCCTAGAGGCCGAGGAGCTCACAGCTATGGGCTGGAGGCCCCGGAGAGCTCGGGGGAC
CCCGTTGCTGCTGCTGCTACTACTGCTGCTGCTCTGGCCAGTGCCAGGCGCCGGGGTGCTTCAAGGAC
ATATCCCTGGGCAGCCAGTCACCCCGCACTGGGTCCTGGATGGACAACCCTGGCGCACCGTCAGCCTG
GAGGAGCCGGTCTCGAAGCCAGACATGGGGCTGGTGGCCCTGGAGGCTGAAGGCCAGGAGCTCCTGCT
TGAGCTGGAGAAGAACCACAGGCTGCTGGCCCCAGGATACATAGAAACCCACTACGGCCCAGATGGGC
AGCCAGTGGTGCTGGCCCCCAACCACACGGATCATTGCCACTACCAAGGGCGAGTAAGGGGCTTCCCC
GACTCCTGGGTAGTCCTCTGCACCTGCTCTGGGATGAGTGGCCTGATCACCCTCAGCAGGAATGCCAG
CTATTATCTGCGTCCCTGGCCACCCCGGGGCTCCAAGGACTTCTCAACCCACGAGATCTTTCGGATGG
AGCAGCTGCTCACCTGGAAAGGAACCTGTGGCCACAGGGATCCTGGGAACAAAGCGGGCATGACCAGC
CTTCCTGGTGGTCCCCAGAGCAGGGGCAGGCGAGAAGCGCGCAGGACCCGGAAGTACCTGGAACTGTA
CATTGTGGCAGACCACACCCTGTTCTTGACTCGGCACCGAAACTTGAACCACACCACACAGCGTCTCC
TGGAAGTCGCCAACTACGTGGACCAGCTTCTCAGGACTCTGGACATTCAGGTGGCGCTGACCGGCCTG
GAGGTGTGGACCGAGCGGGACCGCAGCCGCGTCACGCAGGACGCCAACGCCACGCTCTGGGCCTTCCT
GCAGTGGCGCCGGGGGCTGTGGGCGCAGCGGCCCCACGACTCCGCGCAGCTGCTCACGGGCCGCGCCT
TCCAGGGCGCCACAGTGGGCCTGGCGCCCGTCGAGGGCATGTGCCGCGCCGAGAGCTCGGGAGGCGTG
AGCACGGACCACTCGGAGCTCCCCATCGGCGCCGCAGCCACCATGGCCCATGAGATCGGCCACAGCCT
CGGCCTCAGCCACGACCCCGACGGCTGCTGCGTGGAGGCTGCGGCCGAGTCCGGAGGCTGCGTCATGG
CTGCGGCCACCGGGCACCCGTTTCCGCGCGTGTTCAGCGCCTGCAGCCGCCGCCAGCTGCGCGCCTTC
TTCCGCAAGGGGGGCGGCGCTTGCCTCTCCAATGCCCCGGACCCCGGACTCCCGGTGCCGCCGGCGCT
CTGCGGGAACGGCTTCGTGGAAGCGGGCGAGGAGTGTGACTGCGGCCCTGGCCAGGAGTGCCGCGACC
TCTGCTGCTTTGCTCACAACTGCTCGCTGCGCCCGGGGGCCCAGTGCGCCCACGGGGACTGCTGCGTG
CGCTGCCTGACGTTTACCTACTGGACGGCTCACCCTGTGCCAGGGGCAGTGGCTACTGCTGGGATGGC
GCATGTCCCACGCTGGAGCAGCAGTGCCAGCAGCTCTGGGGGCCTGGCTCCCACCCAGCTCCCGAGGC
CTGTTTCCAGGTGGTGAACTCTGCGGGAGATGCTCATGGAAACTGCGGCCAGGACAGCGAGGGCCACT
TCCTGCCCTGTGCAGGGAGGGATGCCCTGTGTGGGAAGCTGCAGTGCCAGGGTGGAAAGCCCAGCCTG
CTCGCACCGCACATGGTGCCAGTGGACTCTACCGTTCACCTAGATGGCCAGGAAGTGACTTGTCGGGG
AGCCTTGGCACTCCCCAGTGCCCAGCTGGACCTGCTTGGCCTGGGCCTGGTAGAGCCAGGCACCCAGT
GTGGACCTAGAATGGTGTGCCAGAGCAGGCGCTGCAGGAAGAATGCCTTCCAGGAGCTTCAGCGCTGC
CTGACTGCCTGCCACAGCCACGGGGTTTGCAATAGCAACCATAACTGCCACTGTGCTCCAGGCTGGGC
TCCACCCTTCTGTGACAAGCCAGGCTTTGGTGGCAGCATGGACAGTGGCCCTGTGCAGGCTGAAAACC
ATGACACCTTCCTGCTGGCCATGCTCCTCAGCGTCCTGCTGCCTCTGCTCCCAGGGGCCGGCCTGGCC
TGGTGTTGCTACCGACTCCCAGGAGCCCATCTGCAGCGATGCAGCTGGGGCTGCAGAAGGGACCCTGC
GTGCAGTGGCCCCAAAGATGGCCCACACAGGGACCACCCCCTGGGCGGCGTTCACCCCATGGAGTTGG
GCCCCACAGCCACTGGACAGCCCTGGCCCCTGGACCCTGAGAACTCTCATGAGCCCAGCAGCCACCCT
GAGAAGCCTCTGCCAGCAGTCTCGCCTGACCCCCAAGATCAAGTCCAGATGCCAAGATCCTGCCTCTG
GTGAGAGGTAGCTCCTAAAATGAACAGATTTAAAGACAGGTGGCCACTGACAGCCACTCCAGGAACTT
GAACTGCAGGGGCAGAGCCAGTGAATCACCGGACCTCCAGCACCTGCAGGCAGCTTGGAAGTTTCTTC
CCCGAGTGGAGCTTCGACCCACCCACTCCAGGAACCCAGAGCCACATTAGAAGTTCCTGAGGGCTGGA
GAACACTGCTGGGCACACTCTCCAGCTCAATAAACCATCAGTCCCAGAAGCAAAGGTCACACAGCCCC
TGACCTCCCTCACCAGTGGAGGCTGGGTAGTGCTGGCCATCCCAAAAGGGCTCTGTCCTGGGAGTCTG
GTGTGTCTCCTACATGCAATTTCCACGGACCCAGCTCTGTGGAGGGCATGACTGCTGGCCAGAAGCTA
GTGGTCCTGGGGCCCTATGGTTCGACTGAGTCCACACTCCCCTGGAGCCTGGCTGGCCTCTGCAAACA
AACATAATTTTGGGGACCTTCCTTCCTGTTTCTTCCCACCCTGTCTTCTCCCCTAGGTGGTTCCTGAG
CCCCCACCCCCAATCCCAGTGCTACACCTGAGGTTCTGGAGCTCAGAATCTGACAGCCTCTCCCCCAT
TCTGTGTGTGTCGGGGGGACAGAGGGAACCATTTAAGAAAAGATACCAAAGTAGAAGTCAAAAGAAAG
ACATGTTGGCTATAGGCGTGGTGGCTCATGCCTATAATCCCAGCACTTTGGGAAGCCGGGGTAGGAGG
ATCACCAGAGGCCAGCAGGTCCACACCAGCCTGGGCAACACAGCAAGACACCGCATCTACAGAAAAAT
TTTAAAATTAGCTGGGCGTGGTGGTGTGTACCTGTAGGCCTAGCTGCTCAGGAGGCTGAAGCAGGAGG
ATCACTTGAGCCTGAGTTCAACACTGCAGTGAGCTATGGTGGCACCACTGCACTCCAGCCTGGGTGAC
AGAGCAAGACCCTGTCTCT

NOV14c, CG50367-06

| | | | |
|---|---|---|---|
| Protein Sequence | SEQ ID NO: 154 | 514 aa | MW at 55996.3kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRMEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLTFTYWTAHP
VPGAVATAGMAHVPRWSSSASSSGGLAPTQLPRPVSRW

| | | |
|---|---|---|
| NOV14d, CG50367-07 | SEQ ID NO: 155 | 3139 bp |

| | | |
|---|---|---|
| DNA Sequence | ORF Start: ATG at 37 | ORF Stop: TGA at 1354 |

GCGAGCCGCTGCCTAGAGGCCGAGGAGCTCACAGCTATGGGCTGGAGGCCCCGGAGAGCTCGGGGGAC
CCCGTTGCTGCTGCTGCTACTACTGCTGCTGCTCTGGCCAGTGCCAGGCGCCGGGGTGCTTCAAGGAC
ATATCCCTGGGCAGCCAGTCACCCCGCACTGGGTCCTGGATGGACAACCCTGGCGCACCGTCAGCCTG
GAGGAGCCGGTCTCGAAGCCAGACATGGGGCTGGTGGCCCTGGAGGCTGAAGGCCAGGAGCTCCTGCT
TGAGCTGGAGAAGAACCACAGGCTGCTGGCCCCAGGATACATAGAAACCCACTACGGCCCAGATGGGC
AGCCAGTGGTGCTGGCCCCCAACCACACGGATCATTGCCACTACCAAGGGCGAGTAAGGGGCTTCCCC
GACTCCTGGGTAGTCCTCTGCACCTGCTCTGGGATGAGTGGCCTGATCACCCTCAGCAGGAATGCCAG
CTATTATCTGCGTCCCTGGCCACCCCGGGGCTCCAAGGACTTCTCAACCCACGAGATCTTTCGGATGG
AGCAGCTGCTCACCTGGAAAGGAACCTGTGGCCACAGGGATCCTGGGAACAAAGCGGGCATGACCAGC
CTTCCTGGTGGTCCCCAGAGCAGGGGCAGGCGAGAAGCGCGCAGGACCCGGAAGTACCTGGAACTGTA
CATTGTGGCAGACCACACCCTGTTCTTGACTCGGCACCGAAACTTGAACCACACCAAACAGCGTCTCC
TGGAAGTCGCCAACTACGTGGACCAGCTTCTCAGGACTCTGGACATTCAGGTGGCGCTGACCGGCCTG
GAGGTGTGGACCGAGCGGGACCGCAGCCGCGTCACGCAGGACGCCAACGCCACGCTCTGGGCCTTCCT
GCAGTGGCGCCGGGGACTGTGGGCGCAGCGGCCCCACGACTCCGCGCAGCTGCTCACGACCACTCGGA
GCTCCCCATCGGCGCCGCAGCCACCATGGCCCATGAGATCGGCCACAGCCTCGGCCTCAGCCACGACC
CCGACGGCTGCTGCGTGGAGGCTGCGGCCGAGTCCGGAGGCTGCGTCATGGCTGCGGCCACCGGGCAC
CCGTTTCCGCGCGTGTTCAGCGCCTGCAGCCGCCGCCAGCTGCGCGCCTTCTTCCGCAAGGGGGGCGG
CGCTTGCCTCTCCAATGCCCCGGACCCCGGACTCCCGGTGCCGCCGGCGCTCTGCGGGAACGGCTTCG
TGGAAGCGGGCGAGGAGTGTGACTGCGGCCCTGGCCAGGAGTGCCGCGACCTCTGCTGCTTTGCTCAC
AACTGCTCGCTGCGCCCGGGGGCCCAGTGCGCCCACGGGGACTGCTGCGTGCGCTGCCTGCTGAAGCC
GGCTGGAGCGCTGTGCCGCCAGGCCATGGGTGACTGTGACCTCCCTGAGTTTTGCACGGGCACCTCCT
CCCACTGTCCCCCAGACGTTTACCTACTGGACGGCTCACCCTGTGCCAGGGGCAGTGGCTACTGCTGG
GATGGCGCATGTCCCACGCTGGAGCAGCAGTGCCAGCAGCTCTGGGGGCCTGGCTCCCACCCAGCTCC
CGAGGCCTGTTTCCAGGTGGTGAACTCTGCGGGAGATGCTCATGGAAACTGCGGCCAGGACAGCGAGG
GCCACTTCCTGCCCTGTGCAGGGAGGGATGCCCTGTGTGGGAAGCTGCAGTGCCAGGGTGGAAAGCCC
AGCCTGCTCGCACCGCACATGGTGCCAGTGGACTCTACCGTTCACCTAGATGGCCAGGAAGTGACTTG
TCGGGGAGCCTTGGCACTCCCCAGTGCCCAGCTGGACCTGCTTGGCCTGGGCCTGGTAGAGCCAGGCA
CCCAGTGTGGACCTAGAATGGTGTGCCAGAGCAGGCGCTGCAGGAAGAATGCCTTCCAGGAGCTTCAG
CGCTGCCTGACTGCCTGCCACAGCCACGGGGTTTGCAATAGCAACCATAACTGCCACTGTGCTCCAGG
CTGGGCTCCACCCTTCTGTGACAAGCCAGGCTTTGGTGGCAGCATGGACAGTGGCCCTGTGCAGGCTG
AAAACCATGACACCTTCCTGCTGGCCATGCTCCTCAGCGTCCTGCTGCCTCTGCTCCCAGGGGCCGGC
CTGGCCTGGTGTTGCTACCGACTCCCAGGAGCCCATCTGCAGCGATGCAGCTGGGGCTGCAGAAGGGA
CCCTGCGTGCAGTGGCCCCAAAGATGGCCCACACAGGGACCACCCCCTGGGCGGCGTTCACCCCATGG
AGTTGGGCCCCACAGCCACTGGACAGCCCTGGCCCCTGGACCCTGAGAACTCTCATGAGCCCAGCAGC
CACCCTGAGAAGCCTCTGCCAGCAGTCTCGCCTGACCCCCAAGATCAAGTCCAGATGCCAAGATCCTG
CCTCTGGTGAGAGGTAGCTCCTAAAATGAACAGATTTAAAGACAGGTGGCCACTGACAGCCACTCCAG
GAACTTGAACTGCAGGGGCAGAGCCAGTGAATCACCGGACCTCCAGCACCTGCAGGCAGCTTGGAAGT
TTCTTCCCCGAGTGGAGCTTCGACCCACCCACTCCAGGAACCCAGAGCCACATTAGAAGTTCCTGAGG
GCTGGAGAACACTGCTGGGCACACTCTCCAGCTCAATAAACCATCAGTCCCAGAAGCAAAGGTCACAC
AGCCCCTGACCTCCCTCACCAGTGGAGGCTGGGTAGTGCTGGCCATCCCAAAAGGGCTCTGTCCTGGG
AGTCTGGTGTGTCTCCTACATGCAATTTCCACGGACCCAGCTCTGTGGAGGGCATGACTGCTGGCCAG
AAGCTAGTGGTCCTGGGGCCCTATGGTTCGACTGAGTCCACACTCCCCTGGAGCCTGGCTGGCCTCTG
CAAACAAACATAATTTTGGGGACCTTCCTTCCTGTTTCTTCCCACCCTGTCTTCTCCCCTAGGTGGTT
CCTGAGCCCCCACCCCCAATCCCAGTGCTACACCTGAGGTTCTGGAGCTCAGAATCTGACAGCCTCTC
CCCCATTCTGTGTGTGTCGGGGGGACAGAGGGAACCATTTAAGAAAAGATACCAAAGTAGAAGTCAAA
AGAAAGACATGTTGGCTATAGGCGTGGTGGCTCATGCCTATAATCCCAGCACTTTGGGAAGCCGGGGT
AGGAGGATCAC

NOV14d, CG50367-07

| | | | |
|---|---|---|---|
| Protein Sequence | SEQ ID NO: 156 | 439 aa | MW at 48163.4kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRMEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTTTRSSPSAPQPPWPMRSATASASATTPTAAAWRLRPSP
EAASWLRPPGTRFRACSAPAAAASCAPSSARGAALASPMPRTPDSRCRRRSAGTASWKRARSVTAALA
RSAATSAALLTTARCARGPSAPTGTAACAAC

| | | |
|---|---|---|
| NOV14e, 249356906 | SEQ ID NO: 157 | 1278 bp |

| | | |
|---|---|---|
| DNA Sequence | ORF Start: at 1 | ORF Stop: end of sequence |

AAGCTTGAGCTGGAGAAGAACCACAGGCTGCTGGCCCCAGGATACATAGAAACCCACTACGGCCCAGA
TGGGCAGCCAGTGGTGCTGGCCCCCAACCACACGGATCATTGCCACTACCAAGGGCGAGTAAGGGGCT
TCCCCGACTCCTGGGTAGTCCTCTGCACCTGCTCTGGGATGAGTGGCCTGATCACCCTCAGCAGGAAT
GCCAGCTATTATCTGCGTCCCTGGCCACCCCGGGGCTCCAAGGACTTCTCAACCCACGAGATCTTTCG
GATGGAGCAGCTGCTCACCTGGAAAGGAACCTGTGGCCACAGGGATCCTGGGAACAAAGCGGGCATGA
CCAGCCTTCCTGGTGGTCCCCAGAGCAGGGGCAGGCGAGAAGCGCGCAGGACCCGGAAGTACCTGGAA
CTGTACATTGTGGCAGACCACACCCTGTTCTTGACTCGGCACCGAAACTTGAACCACACCAAACAGCG
TCTCCTGGAAGTCGACAACTACGTGGACCAGCTTCTCAGGACTCTGGACATTCAGGTGGCGCTGACCG
GCCTGGAGGTGTGGACCGAGCGGGACCGCAGCCGCGTCACGCAGGACGCCAACGCCACGCTCTGGGCC
TTCCTGCAGTGGCGCCGGGGACTGTGGGCGCAGCGGCCCCACGACTCCGCGCAGCTGCTCACGGGCCG
CGCCTTCCAGGGCGCCACAGTGGGCCTGGCGCCCGTCGAGGGCATGTGCCGCGCCGAGAGCTCGGGAG
GCGTGAGCACGGACCACTCGGAGCTCCCCATCGGCGCCGCAGCCACCATGGCCCATGAGATCGGCCAC
AGCCTCGGCCTCAGCCACGACCCCGACGGCTGCTGCGTGGAGGCTGCGGCCGAGTCCGGAGGCTGCGT
CATGGCTGCGGCCACCGGGCACCCGTTTCCGCGCGTGTTCAGCGCCTGCAGCCGCCGCCAGCTGCGCG
CCTTCTTCCGCAAGGGGGGCGGCGCTTGCCTCTCCAATGCCCCGGACCCCGGACTCCCGGTGCCGCCG
GCGCTCTGCGGGAACGGCTTCGTGGAAGCGGGCGAGGAGTGTGACTGCGGCCCTGGCCAGGAGTGCCG
CGACCTCTGCTGCTTTGCTCACAACTGCTCGCTGCGCCCGGGGGCCCAGTGCGCCCACGGGGACTGCT
GCGTGCGCTGCCTGCTGAAGCCGGCTGGAGCGCTGTGCCGCCAGGCCATGGGTGACTGTGACCTCCCT
GAGTTTTGCACGGGCACCTCCTCCCACTGTCCCCCAGACGTTTACCTACTCGAG

NOV14e, 249356906

| | | | |
|---|---|---|---|
| Protein Sequence | SEQ ID NO: 158 | 426 aa | MW at 46516.2kD |

KLELEKNHRLLAPGYIETHYGPDGQPVVLAPNHTDHCHYQGRVRGFPDSWVVLCTCSGMSGLITLSRN
ASYYLRPWPPRGSKDFSTHEIFRMEQLLTWKGTCGHRDPGNKAGMTSLPGGPQSRGRREARRTRKYLE
LYIVADHTLFLTRHRNLNHTKQRLLEVDNYVDQLLRTLDIQVALTGLEVWTERDRSRVTQDANATLWA
FLQWRRGLWAQRPHDSAQLLTGRAFQGATVGLAPVEGMCRAESSGGVSTDHSELPIGAAATMAHEIGH
SLGLSHDPDGCCVEAAAESGGCVMAAATGHPFPRVFSACSRRQLRAFFRKGGGACLSNAPDPGLPVPP
ALCGNGFVEAGEECDCGPGQECRDLCCFAHNCSLRPGAQCAHGDCCVRCLLKPAGALCRQAMGDCDLP
EFCTGTSSHCPPDVYLLE

| | | |
|---|---|---|
| NOV14f, CG50367-02 | SEQ ID NO: 159 | 2705 bp |

| | | |
|---|---|---|
| DNA Sequence | ORF Start: ATG at 3 | ORF Stop: TGA at 2688 |

CTATGGGCTGGAGGCCCCGGAGAGCTCGGGGGACCCCGTTGCTGCTGCTGCTACTACTGCTGCTGCTC
TGGCCAGTGCCAGGCGCCGGGGTGCTTCAAGGACATATCCCTGGGCAGCCAGTCACCCCGCACTGGGT
CCTGGATGGACAACCCTGGCGCACCGTCAGCCTGGAGGAGCCGGTCTCGAAGCCAGACATGGGGCTGG
TGGCCCTGGAGGCTGAAGGCCAGGAGCTCCTGCTTGAGCTGGAGAAGAACCACAGGCTGCTGGCCCCA
GGATACATAGAAACCCACTACGGCCCAGATGGGCAGCCAGTGGTGCTGGCCCCCAACCACACGGATCA
TTGCCACTACCAAGGGCGAGTAAGGGGCTTCCCCGACTCCTGGGTAGTCCTCTGCACCTGCTCTGGGA
TGAGTGGCCTGATCACCCTCAGCAGGAATGCCAGCTATTATCTGCGTCCCTGGCCACCCCGGGGCTCC
AAGGACTTCTCAACCCACGAGATCTTTCGGATGGAGCAGCTGCTCACCTGGAAAGGAACCTGTGGCCA
CAGGGATCCTGGGAACAAAGCGGGCATGACCAGCCTTCCTGGTGGTCCCCAGAGCAGGGGCAGGCGAG
AAGCGCGCAGGACCCGGAAGTACCTGGAACTGTACATTGTGGCAGACCACACCCTGTTCTTGACTCGG
CACCGAAACTTGAACCACACCAAACAGCGTCTCCTGGAAGTCGCCAACTACGTGGACCAGCTTCTCAG
GACTCTGGACATTCAGGTGGCGCTGACCGGCCTGGAGGTGTGGACCGAGCGGGACCGCAGCCGCGTCA
CGCAGGACGCCAACGCCACGCTCTGGGCCTTCCTGCAGTGGCGCCGGGGGCTGTGGGCGCAGCGGCCC
CACGACTCCGCGCAGCTGCTCACGGGCCGCGCCTTCCAGGGCGCCACAGTGGGCCTGGCGCCCGTCGA
GGGCATGTGCCGCGCCGAGAGCTCGGGAGGCGTGAGCACGGACCACTCGGAGCTCCCCATCGGCGCCG
CAGCCACCATGGCCCATGAGATCGGCCACAGCCTCGGCCTCAGCCACGACCCCGACGGCTGCTGCGTG
GAGGCTGCGGCCGAGTCCGGAGGCTGCGTCATGGCTGCGGCCACCGGGCACCCGTTTCCGCGCGTGTT
CAGCGCCTGCAGCCGCCGCCAGCTGCGCGCCTTCTTCCGCAAGGGGGGCGGCGCTTGCCTCTCCAATG
CCCCGGACCCCGGACTCCCGGTGCCGCCGGCGCTCTGCGGGAACGGCTTCGTGGAAGCGGGCGAGGAG
TGTGACTGCGGCCCTGGCCAGGAGTGCCGCGACCTCTGCTGCTTTGCTCACAACTGCTCGCTGCGCCC
GGGGGCCCAGTGCGCCCACGGGGACTGCTGCGTGCGCTGCCTGCTGCCGCCGGCTGGAGCGCTGTGCC
GCCAGGCCATGGGTGACTGTGACCTCCCTGAGTTTTGCACGGGCACCTCCTCCCACTGTCCCCCAGAC
GTTTACCTACTGGACGGCTCACCCTGTGCCAAGGGCAGTGGCTACTGCTGGGATGGCGCATGTCCCAC
GCTGGAGCAGCAGTGCCAGCAGCTCTGGGGGCCTGGCTCCCACCCAGCTCCCGAGGCCTGTTTCCAGG
TGGTGAACTCTGCGGGAGATGCTCATGGAAACTGCGGCCAGGACAGCGAGGGCCACTTCCTGCCCTGT
GCAGGGAGGGATGCCCTGTGTGGGAAGCTGCAGTGCCAGGGTGGAAAGCCCAGCCTGCTCGCACCGCA
CATGGTGCCAGTGGACTCTACCGTTCACCTAGATGGCCAGGAAGTGACTTGTCGGGGAGCCTTGGCAC
TCCCCAGTGCCCAGCTGGACCTGCTTGGCCTGGGCCTGGTAGAGCCAGGCACCCAGTGTGGACCTAGA
ATGGTGTGCCAGAGCAGGCGCTGCAGGAAGAATGCCTTCCAGGAGCTTCAGCGCTGCCTGACTGCCTG
CCACAGCCACGGGGTTTGCAATAGCAACCATAACTGCCACTGTGCTCCAGGCTGGGCTCCACCCTTCT
GTGACAAGCCAGGCTTTGGTGGCAGCATGGACAGTGGCCCTGTGCAGGCTGALAACCATGACACCTTC
CTGCTGGCCATGCTCCTCAGCGTCCTGCTGCCTCTGCTCCCAGGCGCCGGCCTGGCCTGGTGTTGCTA
CCGACTCCCAGGAGCCCATCTGCAGCGATGCAGCTGGGGCTGCAGAAGGGACCCTGCGTGCAGTGGCC
CCAAAGATGGCCCACACAGAGACCACCCCCTGGGCGGCGTTCACCCCATGGAGTTGGGCCCCACAGCC
ACTGGACAGCCCTGGCCCCTGGACCCTGAGAACTCTCATGAGCCCAGCAGCCACCCTGAGAAGCCTCT
GCCAGCAGTCTCGCCTGACCCCCAAGGTGGTTCCCTTGCAGCCTGGGGCCCCAGTCCTTTAGGGGACA
ACATATCCTCCTCATTCTCAGCAGATCAAGTCCAGATGCCAAGATCCTGCCTCTGTGGCGAACCCTGG
GGAGGCCACGTGGGAAGGAAAGAGGGCTCTAAGAGGGGAGGCCCCAGACTGGGGGAGAGGCCTGTCTG
GAGCCCAGGATCACCTGGCTGTGCTGCAGAACTGGAGAAGAGAAGCTCAGCAGCAGGGAGCTGGCATG
GGGCCAACAGCAGAAAAGCAGGAGGCACGCAGAAGTGACTGGGAAGCAGGAGG

NOV14f, CG50367-02

| | | | |
|---|---|---|---|
| Protein Sequence | SEQ ID NO: 160 | 895 aa | MW at 96021.0kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGN
SGLITLSRNASYYLRPWPPRGSKDFSTHEIFRNEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLLKPAGALCR
QAMGDCDLPEFCTGTSSHCPPDVYLLDGSPCAKGSGYCWDGACPTLEQQCQQLWGPGSHPAPEACFQV
VNSAGDAHGNCGQDSEGHFLPCAGRDALCGKLQCQGGKPSLLAPHMVPVDSTVHLDGQEVTCRGALAL
PSAQLDLLGLGLVEPGTQCGPRMVCQSRRCRKNAFQELQRCLTACHSHGVCNSNHNCHCAPGWAPPFC
DKPGFGGSMDSGPVQAENHDTFLLAMLLSVLLPLLPGAGLAWCCYRLPGAHLQRCSWGCRRDPACSGP
KDGPHRDHPLGGVHPMELGPTATGQPWPLDPENSHEPSSHPEKPLPAVSPDPQGGSLAAWGPSPLGDN
ISSSFSADQVQMPRSCLCGEPWGGHVGRKEGSKRGGPRLGERPVWSPGSPGCAAELEKRSSAERSWHG
ANSRKAGGTQK

| | | |
|---|---|---|
| NOV14g, CG50367-03 | SEQ ID NO: 161 | 2642 bp |

| | | |
|---|---|---|
| DNA Sequence | ORF Start: ATG at 3 | ORF Stop: TGA at 2625 |

CTATGGGCTGGAGGCCCCGGAGAGCTCGGGGGACCCCGTTGCTGCTGCTGCTACTACTGCTGCTGGGC
TGGCCAGTGCCAGGCGCCGGGGTGCTTCAAGGACATATCCCTGGGCAGCCAGTCACCCCGCACTGGGT
CCTGGATGGACAACCCTGGCGCACCGTCAGCCTGGAGGAGCCGGTCTCGAAGCCAGACATGGGGCTGG
TGGCCCTGGAGGCTGAAGGCCAGGAGCTCCTGCTTGAGCTGGAGAAGAACCACAGGCTGCTGGCCCCA
GGATACATAGAAACCCACTACGGCCCAGATGGGCAGCCAGTGGTGCTGGCCCCCAACCACACGGATCA
TTGCCACTACCAAGGGCGAGTAAGGGGCTTCCCCGACTCCTGGGTAGTCCTCTGCACCTGCTCTGGGA
TGAGTGGCCTGATCACCCTCAGCAGGAATGCCAGCTATTATCTGCGTCCCTGGCCACCCCGGGGCTCC
AAGGACTTCTCAACCCACGAGATCTTTCGGATGGAGCAGCTGCTCACCTGGAAAGGAACCTGTGGCCA
CAGGGATCCTGGGAACAAAGCGGGCATGACCAGCCTTCCTGGTGGTCCCCAGAGCAGGGGCAGGCGAG
AAGCGCGCAGGACCCGGAAGTACCTGGAACTGTACATTGTGGCAGACCACACCCTGTTCTTGACTCGG
CACCGAAACTTGAACCACACCAAACAGCGTCTCCTGGAAGTCGCCAACTACGTGGACCAGCTTCTCAG
GACTCTGGACATTCAGGTGGCGCTGACCGGCCTGGAGGTGTGGACCGAGCGGGACCGCAGCCGCGTCA
CGCAGGACGCCAACGCCACGCTCTGGGCCTTCCTGCAGTGGCGCCGGGGGCTGTGGGCGCAGCGGCCC
CACGACTCCGCGCAGCTGCTCACGGGCCGCGCCTTCCAGGGCGCCACAGTGGGCCTGGCGCCCGTCGA
GGGCATGTGCCGCGCCGAGAGCTCGGGAGGCGTGAGCACGGACCACTCGGAGCTCCCCATCGGCGCCG
CAGCCACCATGGCCCATGAGATCGGCCACAGCCTCGGCCTCAGCCACGACCCCGACGGCTGCTGCGTG
GAGGCTGCGGCCGAGTCCGGAGGCTGCGTCATGGCTGCGGCCACCGGGCACCCGTTTCCGCGCGTGTT
CAGCGCCTGCAGCCGCCGCCAGCTGCGCGCCTTCTTCCGCAAGGGGGGCGGCGCTTGCCTCTCCAATG
CCCCGGACCCCGGACTCCCGGTGCCGCCGGCGCTCTGCGGGAACGGCTTCGTGGAAGCGGGCGAGGAG
TGTGACTGCGGCCCTGGCCAGGAGTGCCGCGACCTCTGCTGCTTTGCTCACAACTGCTCGCTGCGCCC
GGGGGCCCAGTGCGCCCACGGGGACTGCTGCGTGCGCTGCCTGCTGAAGCCGGCTGGAGCGCTGTGCC
GCCAGGCCATGGGTGACTGTGACCTCCCTGAGTTTTGCACGGGCACCTCCTCCCACTGTCCCCCAGAC
GTTTACCTACTGGACGGCTCACCCTGTGCCAAGGGCAGTGGCTACTGCTGGGATGGCGCATGTCCCAC
GCTGGAGCAGCAGTGCCAGCAGCTCTGGGGGCCTGGCTCCCACCCAGCTCCCGAGGCCTGTTTCCAGG
TGGTGAACTCTGCGGGAGATGCTCATGGAAACTGCGGCCAGGACAGCGAGGGCCACTTCCTGCCCTGT
GCAGGGAGGGATGCCCTGTGTGGGAAGCTGCAGTGCCAGGGTGGAAAGCCCAGCCTGCTCGCACCGCA
CATGGTGCCAGTGGACTCTACCGTTCACCTAGATGGCCAGGAAGTGACTTGTCGGGGAGCCTTGGCAC
TCCCCAGTGCCCAGCTGGACCTGCTTGGCCTGGGCCTGGTAGAGCCAGGCACCCAGTGTGGACCTAGA
ATGGTGTGCCAGAGCAGGCGCTGCAGGAAGAATGCCTTCCAGGAGCTTCAGCGCTGCCTGACTGCCTG
CCACAGCCACGGGGTTTGCAATAGCAACCATAACTGCCACTGTGCTCCAGGCTGGGCTCCACCCTTCT
GTGACAAGCCAGGCTTTGGTGGCAGCATGGACAGTGGCCCTGTGCAGGCTGAAAACCATGACACCTTC
CTGCTGGCCATGCTCCTCAGCGTCCTGCTGCCTCTGCTCCCAGGCGCCGGCCTGGCCTGGTGTTGCTA
CCGACTCCCAGGAGCCCATCTGCAGCGATGCAGCTGGGGCTGCAGAAGGGACCCTGCGTGCAGTGGCC
CCAAAGATGGCCCACACAGAGACCACCCCCTGGGCGGCGTTCACCCCATGGAGTTGGGCCCCACAGCC
ACTGGACAGCCCTGGCCCCTGGACCCTGAGAACTCTCATGAGCCCAGCAGCCACCCTGAGAAGCCTCT
GCCAGCAGTCTCGCCTGACCCCCAAGCAGATCAAGTCCAGATGCCAAGATCCTGCCTCTGTGGCGAAC
CCTGGGGAGGCCACGTGGGAAGGAAAGAGGGCTCTAAGAGGGGAGGCCCCAGACTGGGGGAGAGGCCT
GTCTGGAGCCCAGGATCACCTGGCTGTGCTGCAGAACTGGAGAAGAGAAGCTCAGCAGAAAGGAGCTG
GCATGGGGCCAACAGCAGAAAAGCAGGAGGCACGCAGAAGTGACTGGGAAGCAGGAGG

NOV14g, CG50367-03

| | | | |
|---|---|---|---|
| Protein Sequence | SEQ ID NO: 162 | 874 aa | MW at 94031.9kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRNEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLLKPAGALCR
QAMGDCDLPEFCTGTSSHCPPDVYLLDGSPCAKGSGYCWDGACPTLEQQCQQLWGPGSHPAPEACFQV
VNSAGDAHGNCGQDSEGHFLPCAGRDALCGKLQCQGGKPSLLAPHMVPVDSTVHLDGQEVTCRGALAL
PSAQLDLLGLGLVEPGTQCGPRMVCQSRRCRKNAFQELQRCLTACHSHGVCNSNHNCHCAPGWAPPFC
DKPGFGGSMDSGPVQAENHDTFLLANLLSVLLPLLPGAGLAWCCYRLPGAHLQRCSWGCRRDPACSGP
KDGPHRDHPLGGVHPMELGPTATGQPWPLDPENSHEPSSHPEKPLPAVSPDPQADQVQMPRSCLCGEP
WGGHVGRKEGSKRGGPRLGERPVWSPGSPGCAAELEKRSSAERSWHGANSRKAGGTQK

| | | |
|---|---|---|
| NOV14h, CG50367-04 | SEQ ID NO: 163 | 3468 bp |

| | | |
|---|---|---|
| DNA Sequence | ORF Start: ATG at 37 | ORF Stop: TGA at 2473 |

GCGAGCCGCTGCCTAGAGGCCGAGGAGCTCACAGCTATGGGCTGGAGGCCCCGGAGAGCTCGGGGGAC
CCCGTTGCTGCTGCTGCTACTACTGCTGCTGCTCTGGCCAGTGCCAGGCGCCGGGGTGCTTCAAGGAC
ATATCCCTGGGCAGCCAGTCACCCCGCACTGGGTCCTGGATGGACAACCCTGGCGCACCGTCAGCCTG
GAGGAGCCGGTCTCGAAGCCAGACATGGGGCTGGTGGCCCTGCAGGCTGAAGGCCAGGAGCTCCTGCT
TGAGCTGGAGAAGAACCACAGGCTGCTGGCCCCAGGATACATAGAAACCCACTACGGCCCAGATGGGC
AGCCAGTGGTGCTGGCCCCCAACCACACGGATCATTGCCACTACCAAGGGCGAGTAAGGGGCTTCCCC
GACTCCTGGGTAGTCCTCTGCACCTGCTCTGGGATGAGTGGCCTGATCACCCTCAGCAGGAATGCCAG
CTATTATCTGCGTCCCTGGCCACCCCGGGGCTCCAAGGACTTCTCAACCCACGAGATCTTTCGGATGG
AGCAGCTGCTCACCTGGAAAGGAACCTGTGGCCACAGGGATCCTGGGAACAAAGCGGGCATGACCAGC
CTTCCTGGTGGTCCCCAGAGCAGGGGCAGGCGAGAAGCGCGCAGGACCCGGAAGTACCTGGAACTGTA
CATTGTGGCAGACCACACCCTGTTCTTGACTCGGCACCGAAACTTGAACCACACCAAACAGCGTCTCC
TGGAAGTCGCCAACTACGTGGACCAGCTTCTCAGGACTCTGGACATTCAGGTGGCGCTGACCGGCCTG
GAGGTGTGGACCGAGCGGGACCGCAGCCGCGTCACGCAGGACGCCAACGCCACGCTCTGGGCCTTCCT
GCAGTGGCGCCGGGGGCTGTGGGCGCAGCGGCCCCACGACTCCGCGCAGCTGCTCACGGGCCGCGCCT
TCCAGGGCGCCACAGTGGGCCTGGCGCCCGTCGAGGGCATGTGCCGCGCCGAGAGCTCGGGAGGCGTG
AGCACGGACCACTCGGAGCTCCCCATCGGCGCCGCAGCCACCATGGCCCATGAGATCGGCCACAGCCT
CGGCCTCAGCCACGACCCCGACGGCTGCTGCGTGGAGGCTGCGGCCGAGTCCGGAGGCTGCGTCATGG
CTGCGGCCACCGGGCACCCGTTTCCGCGCGTGTTCAGCGCCTGCAGCCGCCGCCAGCTGCGCGCCTTC
TTCCGCAAGGGGGGCGGCGCTTGCCTCTCCAATGCCCCGGACCCCGGACTCCCGGTGCCGCCGGCGCT
CTGCGGGAACGGCTTCGTGGAAGCGGGCGAGGAGTGTGACTGCGGCCCTGGCCAGGAGTGCCGCGACC
TCTGCTGCTTTGCTCACAACTGCTCGCTGCGCCCGGGGGCCCAGTGCGCCCACGGGGACTGCTGCGTG
CGCTGCCTGCTGAAGCCGGCTGGAGCGCTGTGCCGCCAGGCCATGGGTGACTGTGACCTCCCTGAGTT
TTGCACGGGCACCTCCTCCCACTGTCCCCCAGACGTTTACCTACTGGACGGCTCACCCTGTGCCAGGG
GCAGTGGCTACTGCTGGGATGGCGCATGTCCCACGCTGGAGCAGCAGTGCCAGCAGCTCTGGGGGCCT
GGCTCCCACCCAGCTCCCGAGGCCTGTTTCCAGGTGGTGAACTCTGCGGGAGATGCTCATGGAAACTG
CGGCCAGGACAGCGAGGGCCACTTCCTGCCCTGTGCAGGGAGGGATGCCCTGTGTGGGAAGCTGCAGT
GCCAGGGTGGAAAGCCCAGCCTGCTCGCACCGCACATGGTGCCAGTGGACTCTACCGTTCACCTAGAT
GGCCAGGAAGTGACTTGTCGGGGAGCCTTGGCACTCCCCAGTGCCCAGCTGGACCTGCTTGGCCTGGG
CCTGGTAGAGCCAGGCACCCAGTGTGGACCTAGAATGGTGTGCCAGAGCAGGCGCTGCAGGAAGAATG
CCTTCCAGGAGCTTCAGCGCTGCCTGACTGCCTGCCACAGCCACGGGGTTTGCAATAGCAACCATAAC
TGCCACTGTGCTCCAGGCTGGGCTCCACCCTTCTGTGACAAGCCAGGCTTTGGTGGCAGCATGGACAG
TGGCCCTGTGCAGGCTGAAAACCATGACACCTTCCTGCTGGCCATGCTCCTCAGCGTCCTGCTGCCTC
TGCTCCCAGGGGCCGGCCTGGCCTGGTGTTGCTACCGACTCCCAGGAGCCCATCTGCAGCGATGCAGC
TGGGGCTGCAGAAGGGACCCTGCGTGCAGTGGCCCCAAAGATGGCCCACACAGGGACCACCCCCTGGG
CGGCGTTCACCCCATGGAGTTGGGCCCCACAGCCACTGGACAGCCCTGGCCCCTGGACCCTGAGAACT
CTCATGAGCCCAGCAGCCACCCTGAGAAGCCTCTGCCAGCAGTCTCGCCTGACCCCCAAGATCAAGTC
CAGATGCCAAGATCCTGCCTCTGGTGAGAGGTAGCTCCTAAAATGAACAGATTTAAAGACAGGTGGCC
ACTGACAGCCACTCCAGGAACTTGAACTGCAGGGGCAGAGCCAGTGAATCACCGGACCTCCAGCACCT
GCAGGCAGCTTGGAAGTTTCTTCCCCGAGTGGAGCTTCGACCCACCCACTCCAGGAACCCAGAGCCAC
ATTAGAAGTTCCTGAGGGCTGGAGAACACTGCTGGGCACACTCTCCAGCTCAATAAACCATCAGTCCC
AGAAGCAAAGGTCACACAGCCCCTGACCTCCCTCACCAGTGGAGGCTGGGTAGTGCTGGCCATCCCAA
AAGGGCTCTGTCCTGGGAGTCTGGTGTGTCTCCTACATGCAATTTCCACGGACCCAGCTCTGTGGAGG
GCATGACTGCTGGCCAGAAGCTAGTGGTCCTGGGGCCCTATGGTTCGACTGAGTCCACACTCCCCTGG
AGCCTGGCTGGCCTCTGCAAACAAACATAATTTTGGGGACCTTCCTTCCTGTTTCTTCCCACCCTGTC
TTCTCCCCTAGGTGGTTCCTGAGCCCCCACCCCCAATCCCAGTGCTACACCTGAGGTTCTGGAGCTCA
GAATCTGACAGCCTCTCCCCCATTCTGTGTGTGTCGGGGGGACAGAGGGAACCATTTAAGAAAAGATA
CCAAAGTAGAAGTCAAAAGAAAGACATGTTGGCTATAGGCGTGGTGGCTCATGCCTATAATCCCAGCA
CTTTGGGAAGCCGGGGTAGGAGGATCACCAGAGGCCAGCAGGTCCACACCAGCCTGGGCAACACAGCA
AGACACCGCATCTACAGAAAAATTTTAAAATTAGCTGGGCGTGGTGGTGTGTACCTGTAGGCCTAGCT
GCTCAGGAGGCTGAAGCAGGAGGATCACTTGAGCCTGAGTTCAACACTGCAGTGAGCTATGGTGGCAC
CACTGCACTCCAGCCTGGGTGACAGAGCAAGACCCTGTCTCTAAAATAAATTTTAAAAAGACATATTA

NOV14h, CG50367-04

| | | | |
|---|---|---|---|
| Protein Sequence | SEQ ID NO: 164 | 812 aa | MW at 87666.9kD |

MGWRPRRARGTPLLLLLLLLLLWPVPGAGVLQGHIPGQPVTPHWVLDGQPWRTVSLEEPVSKPDMGLV
ALEAEGQELLLELEKNHRLLAPGYIETHYGPDGQPVVLAPNHTDHCHYQGRVRGFPDSWVVLCTCSGM
SGLITLSRNASYYLRPWPPRGSKDFSTHEIFRMEQLLTWKGTCGHRDPGNKAGMTSLPGGPQSRGRRE
ARRTRKYLELYIVADHTLFLTRHRNLNHTKQRLLEVANYVDQLLRTLDIQVALTGLEVWTERDRSRVT
QDANATLWAFLQWRRGLWAQRPHDSAQLLTGRAFQGATVGLAPVEGMCRAESSGGVSTDHSELPIGAA
ATMAHEIGHSLGLSHDPDGCCVEAAAESGGCVMAAATGHPFPRVFSACSRRQLRAFFRKGGGACLSNA
PDPGLPVPPALCGNGFVEAGEECDCGPGQECRDLCCFAHNCSLRPGAQCAHGDCCVRCLLKPAGALCR
QAMGDCDLPEFCTGTSSHCPPDVYLLDGSPCARGSGYCWDGACPTLEQQCQQLWGPGSHPAPEACFQV
VNSAGDAHGNCGQDSEGHFLPCAGRDALCGKLQCQGGKPSLLAPHMVPVDSTVHLDGQEVTCRGALAL
PSAQLDLLGLGLVEPGTQCGPRIVIVCQSRRCRKNAFQELQRCLTACHSHGVCNSNHNCHCAPGWAPPFC
DKPGFGGSMDSGPVQAENHDTFLLAMLLSVLLPLLPGAGLAWCCYRLPGAHLQRCSWGCRRDPACSGP
KDGPHRDHPLGGVHPMELGPTATGQPWPLDPENSHEPSSHPEKPLPAVSPDPQDQVQMPRSCLW

A ClustalW comparison of the above protein sequences yields the following sequence alignment shown in Table 14B.

TABLE 14B
Comparison of the NOV14 protein sequences.

| | |
|---|---|
| NOV14a | MGWRPRRARGTPLLLLLLLLLLWPVPGAGVLQGHIPGOPVTPHWVLDGQPWRTVSLEEPV |
| NOV14b | MGWRPRRARGTPLLLLLLLLLLWPVPGAGVLQGHIPGQPVTPHWVLDGQPWRTVSLEEPV |
| NOV14c | MGWRPRRARGTPLLLLLLLLLLWPVPGAGVLQGHIPGQPVTPHWVLDGQPWRTVSLEEPV |
| NOV14d | MGWRPRRARGTPLLLLLLLLLLWPVPGAGVLQGHIPGQPVTPHWVLDGQPWRTVSLEEPV |
| NOV14e | ------------------------------------------------------------- |
| NOV14f | MGWRPRRARGTPLLLLLLLLLLWPVPGAGVLQGHIPGQPVTPHWVIDGQPWRTVSLEEPV |
| NOV14g | MGWRPRRARGTPLLLLLLLLLLWPVPGAGVLQGHIPGQPVTPHWVLDGQPWRTVSLEEPV |
| NOV14h | MGWRPRRARGTPLLLLLLLLLLWPVPGAGVLQGHIPGQPVTPHWVLDGQPWRTVSLEEPV |
| NOV14a | SKPDMGLVALEAEGQELLLELEKNHRLLAPGYIETHYGPDGQPVVLAPNHTDHCHYQGRV |
| NOV14b | SKPDMGLVALEAEGQELLLELEKNHRLLAPGYIETHYGPDGQPVVLAPNHTDHCHYQGRV |
| NOV14c | SKPDMGLVALEAEGQELLLELEKNHRLLAPGYIETHYGPDGQPVVLAPNHTDHCHYQGRV |
| NOV14d | SKPDMGLVALEAEGQELLLELEKNHRLLAPGYIETHYGPDGQPVVLAPNHTDHCHYQGRV |
| NOV14e | -----------------KLELEKNHRLLAPGYIETHYGPDGQPVVLAPNHTDHCHYQGRV |
| NOV14f | SKPDMGLVALEAEGQELLLELEKNHRLLAPGYIETHYGPDGQPVVLAPNHTDHCHYQGRV |
| NOV14g | SKPDMGLVALEAEGQELLLELEKNHRLLAPGYIETHYGPDGQPVVLAPNHTDHCHYQGRV |
| NOV14h | SKPDMGLVALEAEGQELLLELEKNHRLLAPGYIETHYGPDGQPVVLAPNHTDHCHYQGRV |
| NOV14a | RGFPDSWVVLCTCSGMSGLITLSRNASYYLRPWPPRGSKDFSTHEIFRMEQLLTWKGTCG |
| NOV14b | RGFPDSWVVLCTCSGMSGLITLSRNASYYLRPWPPRGSKDFSTHEIFRMEQLLTWKGTCG |
| NOV14c | RGFPDSWVVLCTCSGMSGLITLSRNASYYLRPWPPRGSKDFSTHEIFRMEQLLTWKGTCG |
| NOV14d | RGFPDSWVVLCTCSGMSGLITLSRNASYYLRPWPPRGSKDFSTHEIFRMEQLLTWKGTCG |
| NOV14e | RGFPDSWVVLCTCSGMSGLITLSRNASYYLRPWPPRGSKDFSTHEIFRMEQLLTWKGTCG |
| NOV14f | RGFPDSWVVLCTCSGMSGLITLSRNASYYLRPWPPRGSKDFSTHEIFRMEQLLTWKGTCG |
| NOV14g | RGFPDSWVVLCTCSGMSGLITLSRNASYYLRPWPPRGSKDFSTHEIFRNEQLLTWKGTCG |
| NOV14h | RGFPDSWVVLCTCSGMSGLITLSRNASYYLRPWPPRGSKDFSTHEIFRMEQLLTWKGTCG |
| NOV14a | HRDPGNKAGMTSLPGGPQSRGRREARRTRKYLELYIVADHTLFLTRHRNLNHTKQRLLEV |
| NOV14b | HRDPGNKAGMTSLPGGPQSRGRREARRTRKYLELYIVADHTLFLTRHRNLNHTKQRLLEV |
| NOV14c | HRDPGNKAGMTSLPGGPQSRGRREARRTRKYLELYIVADHTLFLTRHRNLNHTKQRLLEV |
| NOV14d | HRDPGNKAGMTSLPGGPQSRGRREARRTRKYLELYIVADHTLFLTRHRNLNHTKQRLLEV |
| NOV14e | HRDPGNKAGMTSLPGGPQSRGRREARRTRKYLELYIVADHTLFLTRHRKLNHTKQRLLEV |
| NOV14f | HRDPGNKAGMTSLPGGPQSRGRREARRTRKYLELYIVADHTLFLTRHRNLNHTKQRLLEV |
| NOV14g | HRDPGNKAGMTSLPGGPQSRGRREARRTRKYLELYIVADHTLFLTRHRNLNHTKQRLLEV |
| NOV14h | HRDPGNKAGMTSLPGGPQSRGRREARRTRKYLELYIVADHTLFLTRHRNLNHTKQRLLEV |
| NOV14a | ANYVDQLLRTLDIQVALTGLEVWTERDRSRVTQDANATLWAFLQWRRGLWAQRPHDSAQL |
| NOV14b | ANYVDQLLRTLDIQVALTGLEVWTERDRSRVTQDANATLWAFLQWRRGLWAQRPHDSAQL |
| NOV14c | ANYVDQLLRTLDIQVALTGLEVWTERDRSRVTQDANATLWAFLQWRRGLWAQRPHDSAQL |
| NOV14d | ANYVDQLLRTLDIQVALTGLEVWTERDRSRVTQDANATLWAFLQWRRGLWAQRPHDSAQL |
| NOV14e | DNYVDQLLRTLDIQVALTGLEVWTERDRSRVTQDANATLWAFLQWRRGLWAQRPHDSAQL |
| NOV14f | ANYVDQLLRTLDIQVALTGLEVWTERDRSRVTQDANATLWAFLQWRRGLWAQRPHDSAQL |
| NOV14g | ANYVDQLLRTLDIQVALTGLEVWTERDRSRVTQDANATLWAFLQWRRGLWAQRPHDSAQL |
| NOV14h | ANYVDQLLRTLDIQVALTGLEVWTERDRSRVTQDANATLWAFLQWRRGLWAQRPHDSAQL |
| NOV14a | LTGRAFQGATVGLAPVEGMCRAESSGGVSTDHSELPIGAAATMAHEIGHSLGLSHDPDGC |
| NOV14b | LTGRAFQGATVGLAPVEGMCRAESSGGVSTDHSELPIGAAATMAHEIGHSLGLSHDPDGC |
| NOV14c | LTGRAFQGATVGLAPVEGMCRAESSGGVSTDHSELPIGAAATMAHEIGHSLGLSHDPDGC |
| NOV14d | LTTTRSSPSAP--QPPWPMRSATASASATTPTAAAWRLRPSPEAASWLRPPGTRFRACSA |
| NOV14e | LTGRAFQGATVGLAPVEGMCRAESSGGVSTDHSELPIGAAATMAHEIGHSLGLSHDPDGC |
| NOV14f | LTGRAFQGATVGLAPVEGMCRAESSGGVSTDHSELPIGAAATMAHEIGHSLGLSHDPDGC |
| NOV14g | LTGRAFQGATVGLAPVEGMCRAESSGGVSTDHSELPIGAAATMAHEIGHSLGLSHDPDGC |
| NOV14h | LTGRAFQGATVGLAPVEGMCRAESSGGVSTDHSELPIGAAATMAHEIGHSLGLSHDPDGC |
| NOV14a | CVEAAAESGGCVMAAATGHPFPRVFSACSRRQLRAFFRKGGGACLSNAPDPGLPVPPALC |
| NOV14b | CVEAAAESGGCVMAAATGHPFPRVFSACSRRQLRAFFRKGGGACLSNAPDPGLPVPPALC |
| NOV14c | CVEAAAESGGCVMAAATGHPFPRVFSACSRRQLRAFFRKGGGACLSNAPDPGLPVPPALC |
| NOV14d | PAAAASCAPSSARGAALASPMPRTPDSRCRR------RSAGTASWKRARSVTAALARSAA |
| NOV14e | CVEAAAESGGCVMAAATGHPFPRVFSACSRRQLRAFFRKGGGACLSNAPDPGLPVPPALC |
| NOV14f | CVEAAAESGGCVMAAATGHPFPRVFSACSRRQLRAFFRKGGGACLSNAPDPGLPVPPALC |
| NOV14g | CVEAAAESGGCVMAAATGHPFPRVFSACSRRQLRAFFRKGGGACLSNAPDPGLPVPPALC |
| NOV14h | CVEAAAESGGCVMAAATGHPFPRVFSACSRRQLRAFFRKGGGACLSNAPDPGLPVPPALC |
| NOV14a | GNGFVEAGEECDCGPGQECRDLCCFAHNCSLRPGAQCAHGDCCVRCLLKP-------AGA |
| NOV14b | GNGFVEAGEECDCGPGQECRDLCCFAHNCSLRPGAQCAHGDCCVRCLLKP-------AGA |
| NOV14c | GNGFVEAGEECDCGPGQECRDLCCFAHNCSLRPGAQCAHGDCCVRCLTFTYWTAHPVPGA |
| NOV14d | TSAALLTTARCARGPSAPTGTAACAAC--------------------------------- |
| NOV14e | GNGFVEAGEECDCGPGQECRDLCCFAHNCSLRPGAQCAHGDCCVRCLLKP-------AGA |
| NOV14f | GNGFVEAGEECDCGPGQECRDLCCFAHNCSLRPGAQCAHGDCCVRCLLKP-------AGA |
| NOV14g | GNGFVEAGEECDCGPGQECRDLCCFAHNCSLRPGAQCAHGDCCVRCLLKP-------AGA |
| NOV14h | GNGFVEAGEECDCGPGQECRDLCCFAHNCSLRPGAQCAHGDCCVRCLLKP-------AGA |
| NOV14a | LCRQAMGDCDLPEFCTGTSSHCPPDVYLLDGSPCARGSGYCWDGACPTLEQQCQQLWGPG |
| NOV14b | LCRQAMGDCDLPEFCTGTSSHCPPDVYLLDGSPCARGSGYCWDGACPTLEQQCQQLWGPG |
| NOV14c | VATAGMAHVPRWSSSASSSGGLAPTQLPRPVSRW-------------------------- |
| NOV14d | ------------------------------------------------------------- |
| NOV14e | LCRQAMGDCDLPEFCTGTSSHCPPDVYLLE------------------------------ |
| NOV14f | LCRQAMGDCDLPEFCTGTSSHCPPDVYLLDGSPCAKGSGYCWDGACPTLEQQCQQLWGPG |
| NOV14g | LCRQAMGDCDLPEFCTGTSSHCPPDVYLLDGSPCAKGSGYCWDGACPTLEQQCQQLWGPG |
| NOV14h | LCRQAMGDCDLPEFCTGTSSHCPPDVYLLDGSPCARGSGYCWDGACPTLEQQCQQLWGPG |
| NOV14a | SHPAPEACFQVVNSAGDAHGNCGQDSEGHFLPCAGRDALCGKLQCQGGKPSLLAPHMVPV |
| NOV14b | SHPAPEACFQVVNSAGDAHGNCGQDSEGHFLPCAGRDALCGKLQCQGGKPSLLAPHMVPV |
| NOV14c | ------------------------------------------------------------- |
| NOV14d | ------------------------------------------------------------- |
| NOV14e | ------------------------------------------------------------- |
| NOV14f | SHPAPEACFQVVNSAGDAHGNCGQDSEGHFLPCAGRDALCGKLQCQGGKPSLLAPHMVPV |
| NOV14g | SHPAPEACFQVVNSAGDAHGNCGQDSEGHFLPCAGRDALCGKLQCQGGKPSLLAPHMVPV |
| NOV14h | SHPAPEACFQVVNSAGDAHGNCGQDSEGHFLPCAGRDALCGKLQCQGGKPSLLAPHMVPV |
| NOV14a | DSTVHLDGQEVTCRGALALPSAQLDLLGLGLVEPGTQCGPRNVCQSRRCRKNAFQELQRC |
| NOV14b | DSTVHLDGQEVTCRGALALPSAQLDLLGLGLVESGTQCGPRMVCN--------------- |
| NOV14c | ------------------------------------------------------------- |
| NOV14d | ------------------------------------------------------------- |
| NOV14e | ------------------------------------------------------------- |
| NOV14f | DSTVHLDGQEVTCRGALALPSAQLDLLGLGLVEPGTQCGPRMVCQSRRCRKNAFQELQRC |
| NOV14g | DSTVHLDGQEVTCRGALALPSAQLDLLGLGLVEPGTQCGPRMVCQSRRCRKNAFQELQRC |
| NOV14h | DSTVHLDGQEVTCRGALALPSAQLDLLGLGLVEPGTQCGPRMVCQSRRCRKNAFQELQRC |
| NOV14a | LTACHSHGVCNSNHNCHCAPGWAPPFCDKPGFGGSMDSGPVQAENHDTFLLAMLLSVLLP |
| NOV14b | -----------SNHNCHCAPGWAPPFCDKPGFGGSMDSGPVQAENHDTFLLAMLLSVLLP |
| NOV14c | ------------------------------------------------------------- |
| NOV14d | ------------------------------------------------------------- |
| NOV14e | ------------------------------------------------------------- |
| NOV14f | LTACHSHGVCNSNHNCHCAPGWAPPFCDKPGFGGSMDSGPVQAENHDTFLLAMLLSVLLP |
| NOV14g | LTACHSHGVCNSNHNCHCAPGWAPPFCDKPGFGGSMDSGPVQAENHDTFLLAMLLSVLLP |
| NOV14h | LTACHSHGVCNSNHNCHCAPGWAPPFCDKPGFGGSMDSGPVQAENHDTFLLAMLLSVLLP |
| NOV14a | LLPGAGLAWCCYRLPGAHLQRCSWGCRRDPACSGPKDGPHRDHPLGGVHPMELGPTATGQ |
| NOV14b | LLPGAGLAWCCYRLPGAHLQRCSWGCRRDPACSGPKDGPNRDHPLGGVHPMELGPTATGQ |
| NOV14c | ------------------------------------------------------------- |
| NOV14d | ------------------------------------------------------------- |
| NOV14e | ------------------------------------------------------------- |
| NOV14f | LLPGAGLAWCCYRLPGAHLQRCSWGCRRDPACSGPKDGPHRDHPLGGVHPMELGPTATGQ |
| NOV14g | LLPGAGLAWCCYRLPGAHLQRCSWGCRRDPACSGPKDGPHRDHPLGGVHPMELGPTATGQ |
| NOV14h | LLPGAGLAWCCYRLPGAHLQRCSWGCRRDPACSGPKDGPHRDHPLGGVHPMELGPTATGQ |
| NOV14a | PWPLAPGAPADHIHNIYPPPFLPDPENSHEPSSHPEKPLPAVSPDPQGGSLAAWGPSPLG |
| NOV14b | PWPLDP-------------------ENSHEPSSHPEKPLPAVSPDPQ------------- |
| NOV14c | ------------------------------------------------------------- |
| NOV14d | ------------------------------------------------------------- |
| NOV14e | ------------------------------------------------------------- |
| NOV14f | PWPLDP-------------------ENSHEPSSHPEKPLPAVSPDPQGGSLAAWGPSPLG |
| NOV14g | PWPLDP-------------------ENSHEPSSHPEKPLPAVSPDPQ------------- |
| NOV14h | PWPLDP-------------------ENSHEPSSHPEKPLPAVSPDPQ------------- |
| NOV14a | DNISSSFSADQVQMPRSCLCGEPWGGHVGRKEGSKRGGPRLGERPVWSPGSPGCAAELEK |
| NOV14b | --------ADQVQMPRSCLW---------------------------------------- |
| NOV14c | ------------------------------------------------------------- |
| NOV14d | ------------------------------------------------------------- |
| NOV14e | ------------------------------------------------------------- |
| NOV14f | DNISSSFSADQVQMPRSCLCGEPWGGHVGRKEGSKRGGPRLGERPVWSPGSPGCAAELEK |
| NOV14g | --------ADQVQMPRSCLCGEPWGGHVGRKEGSKRGGPRLGERPVWSPGSPGCAAELEK |
| NOV14h | ---------DQVQMPRSCLW---------------------------------------- |
| NOV14a | RSSAERSWHGANSRKAGGTQK |
| NOV14b | --------------------- |
| NOV14c | --------------------- |
| NOV14d | --------------------- |
| NOV14e | --------------------- |
| NOV14f | RSSAERSWHGANSRKAGGTQK |
| NOV14g | RSSAERSWHGANSRKAGGTQK |
| NOV14h | --------------------- |
| NOV14a | (SEQ ID NO: 150) |
| NOV14b | (SEQ ID NO: 152) |
| NOV14C | (SEQ ID NO: 154) |
| NOV14d | (SEQ ID NO: 156) |
| NOV14e | (SEQ ID NO: 158) |
| NOV14f | (SEQ ID NO: 160) |
| NOV14g | (SEQ ID NO: 162) |
| NOV14h | (SEQ ID NO: 164) |

Further analysis of the NOV14a protein yielded the following properties-shown in Table 14C.

TABLE 14C
Protein Sequence Properties NOV14a

SignalP analysis: Cleavage site between residues 30 and 31
PSORT II analysis:

| | |
|---|---|
| PSG: | a new signal peptide prediction method |
| | N-region: length 9; pos.chg 4; neg.chg 0 |
| | H-region: length 37; peak value 11.70 |
| | PSG score: 7.30 |
| GvH: | von Heijne's method for signal seq. recognition |
| | GvH score (threshold: −2.1): 5.85 |
| | possible cleavage site: between 27 and 28 |

>>> Seems to have a cleavable signal peptide (1 to 27)

| ALOM: | Klein et al's method for TM region allocation | |
|---|---|---|
| | Init position for calculation: 28 | |
| | Tentative number of TMS(s) for the threshold 0.5: 2 | |
| | Number of TMS(s) for threshold 0.5: 1 | |
| | INTEGRAL | Likelihood = −6.16 Transmembrane 702-718 |
| | PERIPHERAL | Likelihood = 0.63 (at 126) |

| | ALOM score: −6.16 (number of TMSs: 1) |
|---|---|
| MTOP: | Prediction of membrane topology (Hartmann et al.) |
| | Center position for calculation: 13 |
| | Charge difference: −5.0 C(0.0) − N(5.0) |
| | N >= C: N-terminal side will be inside |

>>> membrane topology: type 1a (cytoplasmic tail 719 to 914)
MITDISC: discrimination of mitochondrial targeting seq

| | | | |
|---|---|---|---|
| R content: | 4 | Hyd Moment(75): | 2.69 |
| Hyd Moment(95): | 8.42 | G content: | 6 |
| D/E content: | 1 | S/T content: | 2 |
| Score: −3.80 | | | |

| | |
|---|---|
| Gavel: | prediction of cleavage sites for mitochondrial preseq |
| | R-2 motif at 19 ARG\|TP |

NUCDISC: discrimination of nuclear localization signals

pat4: RPRR (4) at 4
pat7: PRRARGT (5) at 5
pat7: PQSRGRR (3) at 197
bipartite: none
content of basic residues: 8.9%
NLS Score: 0.47

KDEL: ER retention motif in the C-terminus: none
ER Membrane Retention Signals:

XXRR-like motif in the N-terminus: GWRP

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 70.6 |
| COIL: | Lupas's algorithm to detect coiled-coil regions |
| | total: 0 residues |

Final Results (k = 9/23):

44.4%: extracellular, including cell wall
22.2%: Golgi
22.2%: endoplasmic reticulum
11.1%: plasma membrane

>> prediction for CG50367-01 is exc (k = 9)

A search of the NOV14a protein against the Geneseq database, a proprietary database that contains sequences published in patents and patent publication, yielded several homologous proteins shown in Table 14D.

TABLE 14D
Geneseq Results for NOV14a

| | | NOV14a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABJ10597 | Human novel protein NOV7a | 1 . . . 914 | 914/914 (100%) | 0.0 |
| | SEQ ID NO: 22 - Homo sapiens, | 1 . . . 914 | 914/914 (100%) | |
| | 914 aa. [WO200259315-A2, 01- | | | |
| | AUG-2002] | | | |
| ABJ10598 | Human novel protein NOV7b | 1 . . . 914 | 894/914 (97%) | 0.0 |
| | SEQ ID NO: 24 - Homo sapiens, | 1 . . . 895 | 895/914 (97%) | |
| | 895 aa. [WO200259315-A2, 01- | | | |
| | AUG-2002] | | | |
| ABJ10599 | Human novel protein NOV7c | 1 . . . 914 | 873/914 (95%) | 0.0 |
| | SEQ ID NO: 26 - Homo sapiens, | 1 . . . 874 | 874/914 (95%) | |
| | 874 aa. [WO200259315-A2, 01- | | | |
| | AUG-2002] | | | |
| ABU58632 | Human PRO polypeptide #233 - | 1 . . . 852 | 812/852 (95%) | 0.0 |
| | Homo sapiens, 813 aa. | 1 . . . 812 | 812/852 (95%) | |
| | [US2003027272-A1, 06-FEB- | | | |
| | 2003] | | | |
| ABU57163 | Human PRO polypeptide #233 - | 1 . . . 852 | 812/852 (95%) | 0.0 |
| | Homo sapiens, 813 aa. | 1 . . . 812 | 812/852 (95%) | |
| | [US2003027280-A1, 06-FEB- | | | |
| | 2003] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BZ11 | ADAM 33 precursor (EC | 1 . . . 852 | 812/852 (95%) | 0.0 |
| | 3.4.24.-) (A disintegrin and | 1 . . . 812 | 812/852 (95%) | |
| | metalloproteinase domain 33) - | | | |
| | Homo sapiens (Human), 813 aa. | | | |
| Q8N0W6 | A disintegrin and | 1 . . . 852 | 811/852 (95%) | 0.0 |
| | metalloprotease domain 33 - | 1 . . . 811 | 811/852 (95%) | |
| | Homo sapiens (Human), 812 aa. | | | |
| CAC33153 | Sequence 1 from Patent | 1 . . . 661 | 661/661 (100%) | 0.0 |
| | WO0109293 - Homo sapiens | 1 . . . 661 | 661/661 (100%) | |
| | (Human), 802 aa. | | | |
| Q8R5G5 | Metalloprotease/disintegrin | 1 . . . 805 | 571/806 (70%) | 0.0 |
| | ADAM33 - Mus musculus | 1 . . . 784 | 632/806 (77%) | |
| | (Mouse), 797 aa. | | | |
| Q923W9 | ADAM 33 precursor (EC | 1 . . . 805 | 570/806 (70%) | 0.0 |
| | 3.4.24.-) (A disintegrin and | 1 . . . 784 | 631/806 (77%) | |
| | metalloproteinase domain 33) - | | | |
| | Mus musculus (Mouse), 797 aa. | | | |

PFam analysis indicates that the NOV14a protein contains the domains shown in the Table 14F.

TABLE 14F
Domain Analysis of NOV14a

| | NOV14a | Identities/ | |
|---|---|---|---|
| | Match | Similarities | Expect |
| Pfam Domain | Region | for the Matched Region | Value |
| Pep_M12B_propep | 80 . . . 198 | 44/122 (36%) | 1.3e−31 |
| | | 93/122 (76%) | |
| Reprolysin | 210 . . . 409 | 84/206 (41%) | 3e−88 |
| | | 163/206 (79%) | |
| disintegrin | 426 . . . 501 | 38/76 (50%) | 2.5e−23 |
| | | 52/76 (68%) | |
| EGF | 653 . . . 680 | 9/47 (19%) | 0.43 |
| | | 20/47 (43%) | |

### Example 15

The NOV15 clone was analyzed, and the nucleotide and encoded polypeptide sequences are shown in Table 15A.

TABLE 15A
NOV15 Sequence Analysis

| | | |
|---|---|---|
| NOV15a, CG50718-02 | SEQ ID NO: 165 | 6881 bp |

| | | |
|---|---|---|
| DNA Sequence | ORF Start: ATG at 16 | ORF Stop: TAA at 6859 |

AAAAATGTAATAAAGATGGATTTTCTTATCATTTTTCTTTTACTTTTTATTGGGACTTCAGAGACACA
GGTAGATGTTTCCAATGTCGTTCCTGGTACTAGGTACGATATAACCATCTCTTCAATTTCTACAACAT
ACACCTCACCTGTTACTAGAATAGGGGCTTCTAATGAACCAGGGCCTCCAGTCTTCCTAGCCGGGGAA
AGAGTCGGATCTGCTGGGATTCTTCTGTCTTGGAATACACCACCTAATCCAAATGGAAGGATTATATC
TTACATTGTCAAATATAAGGAAGTTTGTCCGTGGATGCAAACAGTATATACACAAGTCAGATCAAAGC
CAGACAGTCTGGAAGTTCTTCTTACTAATCTTAATCCTGGAACAACATATGAAATTAAGGTAGCTGCT
GAAAACAGTGCTGGCATTGGAGTGTTTAGTGATCCATTTCTCTTCCAAACTGCAGAAAGTGCTCCAGG
AAAAGTGGTGGATTTCACAGGTGAGGCTGTCCCGTTCAGCAGTAAGCTGATGTGGTATACCTCGGCAA
CCAAAAAAAAAATTACCAGCTTCAAGATTAGTGTCAAGCATAACAGAAGTGGGATAGTAGTGAAAGAA
GTGTCAATCAGAGTGGAGTGCATTTTAAGTGCTTCCCTTCCTTTGCACTGCAACGAGAATAGTGAATC
TTTTTTATGGAGTACAGCCAGCCCTTCTCCAACCCTTGGTAGAGTTACACCTCCATCGCGTACCACAC
ATTCATCAAGCACGTTGACACAGAATGAGATCAGCTCTGTGAAAGAGCCTATCAGTTTTGTAGTGACA
CACTTGAGACCTTATACAACATATCTTTTTGAAGTTTCAGCTGCTACAACTGAAGCAGGTTATATTGA
TAGTACGATTGTCAGAACACCAGAATCAGTGCCTGAAGGACCACCACAAAACTGCGTAACAGGCAACA
TCACAGGAAAGTCCTTTTCAATTTTATGGGACCCACCAACTATAGTAACAGGGAAATTTAGTTATAGA
GTTGAATTATATGGACCATCAGCAGGTCGCATTTTGGATAACAGCACAAAAGACCTCAAGTTTGCATT
CACTAACCTAACACCATTTACAATGTATGATGTCTATATTGCGGCTGCGGCCAGTGCAGGGACTGGGC
CCAAGTCAAATATTTCAGTATTCACTCCACCAGATGTTCCAGGGGCAGTGTTTGATTTACAACTTGCA
GAGGTAGAATCCACGCAAGTAAGAATTACTTGGAAGAAACCACGACAACCAAATGGAATTATTAACCA
ATACCGAGTGAAAGTGCTAGTTCCAGAGACAGGAATAATTTTGGAAAATACTTTGCTCACTGGAAATA
ATGAGATAAATGACCCCATGGCTCCAGAATTGTGAACATAGTACAGCCCAATGGTAGGATTATATGAG
GGTTCAGCAGAGATGTCGTCTGACCTTCACTCACTTGCTACATTTATATATAACAGCCATCCAGATAA
AAACTTTCCTGCAAGGAATAGAGCTGAAGACCAGACTTCACCAGTTGTAACTACAAGGAATCAGTATA
TTACTGACATTGCAGCTGAACAGCTGACTTATGTTCTTATCAGATTAAGGAGATTTTGGGCTGAGACA
ATGGGGTTTTCTAGATATACAATCATGTCATCTGCAAGCAGGGACAATTTGACTTCCCCAGGCCCTTT
GTCAGCCCAAAATTTCAGAGTTACACATGTTACCATAACAGAAGTATTTTTACACTGGGATCCTCCAG
ATCCTGTATTTTTTCATCATTACCTTATCACTATTTTGGATGTTGAAAACCAATCCAAGAGTATTATT
TTAAGGACATTAAACAGTTTGTCTCTTGTCCTTATAGGGTTAAAGAAATACACAAAATACAAAATGAG
AGTGGCAGCCTCAACCCACGTTGGAGAAAGTTCTTTGTCTGAAGAAAATGACATCTTTGTGAGAACTT
CAGAAGATGAACCGGAATCATCACCTCAAGATGTCGAAGTAATTGATGTTACCGCAGATGAAATAAGG
TTGAAGTGGTCACCACCCGAAAAGCCCAATGGGATCATTATTGCTTATGAAGTGCTATATAAAAATAT
AGATACTTTATATATGAAGAACACATCAACAACAGACATAATATTAAGGAACTTAAGACCTCACACCC
TCTATAACATTTCTGTAAGGTCTTACACCAGATTTGGTCATGGCAATCAGGTATCTTCTTTACTCTCT
GTAAGGACTTCGGAGTCAGTGCCTGATAGTGCACCAGAAAATATCACTTACAAAAATATTTCTTCTGG
AGAGATTGAGCTATCATTCCTTCCCCCAAGTAGTCCCAATGGAATCATACAAAAATATACAATTTATC
TCAAGAGAAGTAATGGAAATGAGGAAAGAACTATAAATACAACCTCTTTAACCCAAAACATTAAAGGT
CTGAAGAAATATACCCAATATATCATTGAGGTGTCTGCTAGTACACTCAAAGGTGAAGGAGTTCGGAG
TGCTCCCCATAAGTATACTGACGGAGGAAGATGCTCCTGATTCTCCCCCCAAGACTTCTCTGTAAAAC
AGTTGTCTGGTGTCACGGTGAAGTTGTCATGGCAACCACCCCTGGAGCCAAATGGAATTATCCTTTAT
TACACAGTTTATGTCTGGAGATCATCATTAAAAACTATTAATGTCACTGAAATCATTGGAGAGTTATC
AGATTTGGATTATAATGTTGAATACAGTGCTTATGTAACAGCTAGCACCAGATTTGGTGATGGGAAAA
CAAGAAGCAATATCATTAGCTTTCAAACACCAGAGGGACCAAGCGATCCTCCCAAAGATGTTTATTAT
GCAAACCTCAGTTCTTCATCAATAATTCTTTTCTGGACACCTCCTTCAAAACCTAATGGGATTATACA
ATATTACTCTGTTTATTACAGAAATACTTCAGGTACTTTTATGCAGAATTTTACACTCCATGAAGTAA
CCAATGACTTTGACAATATGACTGTATCCACAATTATAGATAAACTGACAATATTCAGCTACTATACA
TTTTGGTTAACAGCAAGTACTTCAGTTGGAAATGGGAATAAAAGCAGTGACATCATTGAAGTATACAC
AGATCAAGACGTACCTGAAGGGTTTGTTGGAAACCTGACTTACGAATCCATTTCGTCAACTGCAATAA
ATGTAAGCTGGGTCCCACCGGCTCAACCAAACGGTCTAGTCTTCTACTATGTTTCACTGATCTTACAG
CAGACTCCTCGCCATGTGAGACCACCTCTTGTTACATATGAGAGAAGCATATATTTTGATAATCTGGA
AAAATACACTGATTATATATTAAAAATTACTCCATCAACAGAAAAGGGATTCTCTGATACCTATACTG
CCCAGCTATACATCAAGACTGAAGAAGATATCCCAGAAACTTCACCAATAATCAACACTTTTAAAAAC
CTTTCCTCTACCTCAGTTCTCTTATCATGGGATCCCCCAGTAAAGCCAAATGGTGCAATAATAAGTTA
TGATTTAACTTTACAAGGACCAAATGAAAATTATTCTTTCATTACTTCTGATAATTACATAATATTGG
AAGAGCTTTCACCATTTACATTATATAGCTTTTTTGCTGCCGCAAGAACTAGAAAAGGACTTGGTCCT
TCCAGTATTCTTTTCTTTTACACAGATGAGTCAGTGCCGTTAGCACCTCCACAAAATTTGACTTTAAT
CAACTGTACTTCAGACTTTGTATGGCTGAAATGGAGCCCAAGTCCTCTTCCAGGTGGTATTGTTAAAG
TATATAGTTTTAAAATTCATGAACATGAAACTGACACTATATATTATAAGAATATATCAGGATTTAAA
ACTGAAGCCAAACTTGTTGGACTGGAACCAGTCAGCACCTACTCTATCCGTGTATCTGCGTTCACCAA
AGTTGGAAATGGCAATCAATTTAGTAATGTAGTAAAATTCACAACCCAAGAATCAGTTCCAGATGTCG
TGCAGAATATGCAGTGCATGGCAACTAGCTGGCAGTCAGTTTTAGTGAAATGGGATCCACCCAAAAAG
GCAAATGGAATAATAACGCAGTATATGGTAACAGTTGAAAGGAATTCTACAAAAGTTTCTCCCCAAGA
TCACATGTACACTTTCATAAAGCTTCTTGCCAATACCTCATATGTCTTTAAAGTAAGAGCTTCAACCT
CAGCTGGTGAAGGTGATGAAAGCACATGCCATGTCAGCACACTACCTGAAACAGTTCCCAGTGTTCCC
ACAAATATTGCTTTTTCTGATGTTCAGTCAACTAGTGCAACATTGACATGGATAAGACCTGACACTAT
CCTTGGCTACTTTCAAAATTACAAAATTACCACTCAAGTTCGTGCTCAAAAATGCAAAGAATGGGAAT
CCGAAGAATGTGTTGAATATCAAAAAATTCAATACCTOTATGAAGCTCACTTAACTGAAGAGACAGTA
TATGGATTAAAGAAATTTAGATGGTATAGATTCCAAGTGGCTGCCAGCACCAATGCTGGCTATGGCAA
TGCTTCAAACTGGATTTCTACAAAAACTCTGCCTGGCCCTCCAGATGGTCCTCCTGAAAATGTTCATG
TAGTAGCAACATCACCTTTTAGCATCAGCATAAGCTGGAGTGAACCTGCTGTCATTACTGGACCAACA
TGTTATCTGATTGATGTCAAATCGGTAGATAATGATGAATTTAATATATCCTTCATCAAGTCAAATGA
AGAAAATAAAACCATAGAAATTAAAGATTTAGAAATATTCACAAGGTATTCTGTAGTGATCACTGCAT
TTACTGGGAACATTAGTGCTGCATATGTAGAAGGGAAGTCAAGTGCTGAAATGATTGTTACTACTTTA
GAATCAGCCCCAAAGGACCCACCTAACAACATGACATTTCAGAAGATACCAGATGAAGTTACAAAATT
TCAATTAACGTTCCTTCCTCCTTCTCAACCTAATGGAAATATCCAAGTATATCAAGCTCTGGTTTACC
GAGAAGATGATCCTACTGCTGTCCAGATTCACAACCTCAGTATTATACAGAAAACCAACACATTCGTC
ATTGCAATGCTAGAAGGACTAAAAGGTGGACATACATACAATATCAGTGTTTACGCAGTCAATAGTGC
TGGTGCAGGTCCAAAGGTTCCGATGAGAATAACCATGGATATCAAAGCTCCAGCACGACCAAAAAACA
AACCAACCCCTATTTATGATGCCACAGGAAAACTGCTTGTGACTTCAACAACAATTACAATCAGAATG
CCAATATGTTACTACAGTGATGATCATGGACCAATAAAAAAAATGTACAAGTGCTTGTGAACAGCAGG
AGCTCAGCATGATGGAAATGTAACAAAGTGGTATGATGCATATTTTAATAAAGCAAGGCCATATTTTA
CAAATGAAGGCTTTCCTAACCCTCCATGTACAGAAGGAAAGACAAAGTTTAGTGGCAATGAAGAAATC
TACATCATAGGTGCTGATAATGCATGCATGATTCCTGGCAATGAAGACAAAATTTGCAATGGACCACT
GAAACCAAAAAAGCAATACTTATTTAAATTTAGAGCTACAAATATTATGGGACAATTTACTGACTCTG
ATTATTCTGACCCTGTTAAGACTTTAGGCGAAGGACTTTCAGAAAGAACCGTAGAGATCATTCTTTCC
GTCACTTTGTGTATCCTTTCAATAATTCTCCTTGGAACAGCTATTTTTGCATTTGCAAGAATTCGACA
GAAGCAGAAAGAAGGTGGCACATACTCTCCTCAGGATGCAGAAATTATTGACACTAAATTGAAGCTGG
ATCAGCTCATCACAGTGGCAGACCTGGAACTGAAGGACGAGAGATTAACGCGGCCAATAAGCAAGAAA
TCCTTCCTGCAACATGTTGAAGAGCTTTGCACAAACAACAACCTAAAGTTTCAAGAAGAATTTTCGGA
ATTACGAAAATTTCTTCAGGATCTTTCTTCAACTGATGCTGATCTGCCTTGGAATAGAGCAAAAAACC
GCTTCCCAAACATAAAACCATATAATAATAACAGAGTAAAGCTGATAGCTGACGCTAGTGTTCCAGGT
TCGGATTATATTAATGCCAGCTATATTTCTGGTTATTTATGTCCAAATGAATTTATTGCTACTCAAGG
TCCACTACCAGGAACAGTTGGAGATTTTTGGAGAATGGTGTGGGAAACCAGAGCAAAAACATTAGTAA
TGCTAACACAGTGTTTTGAAAAAGGACGGATCAGATGCCATCAGTATTGGCCAGAGGACAACAAGCCA
GTTACTGTCTTTGGAGATATAGTGATTACAAAGCTAATGGAGGATGTTCAAATAGATTGGACTATCAG
GGATCTGAAAATTGAAAGGCATGGGGATTGCATGACTGTTCGACAGTGTAACTTTACTGCCTGGCCAG
AGCATGGGGTTCCTGAGAACAGCGCCCCTCTAATTCACTTTGTGAAGTTGGTTCGAGCAAGCAGGGCA
CATGACACCACACCTATGATTGTTCACTGCAGTGCTGGAGTTGGAAGAACTGGAGTTTTTATTGCTCT
GGACCATTTAACACAACATATAAATGACCATGATTTTGTGGATATATATGGACTAGTAGCTGAACTGA
GAAGTGAAAGAATGTGCATGGTGCAGAATCTGGCACAGTATATCTTTTTACACCAGTGCATTCTGGAT
CTCTTATCAAATAAGGGAAGTAATCAGCCCATCTGTTTTGTTAACTATTCAGCACTTCAGAAGATGGA
CTCTTTGGACGCCATGGAAGGTGGTGATGTTGAGCTTGAATGGGAAGAAACCACTATGTAAATATTCA
GACCAAAGGATAC

NOV15a, CG50718-02

| | | | |
|---|---|---|---|
| Protein Sequence | SEQ ID NO: 166 | 2281 aa | MW at 255030.3kD |

MDFLIIFLLLFIGTSETQVDVSNVVPGTRYDITISSISTTYTSPVTRIGASNEPGPPVFLAGERVGSA
GILLSWNTPPNPNGRIISYIVKYKEVCPWMQTVYTQVRSKPDSLEVLLTNLNPGTTYEIKVAAENSAG
IGVFSDPFLFQTAESAPGKVVDFTGEAVPFSSKLMWYTSATKKKITSFKISVKHNRSGIVVKEVSIRV
ECILSASLPLHCNENSESFLWSTASPSPTLGRVTPPSRTTHSSSTLTQNEISSVKEPISFVVTHLRPY
TTYLFEVSAATTEAGYIDSTIVRTPESVPEGPPQNCVTGNITGKSFSILWDPPTIVTGKFSYRVELYG
PSAGRILDNSTKDLKFAFTNLTPFTMYDVYIAAETSAGTGPKSNISVFTPPDVPGAVFDLQLAEVEST
QVRITWKKPRQPNGIINQYRVKVLVPETGIILENTLLTGNNEINDPMAPEIVNIVQPMVGLYEGSAEM
SSDLHSLATFIYNSHPDKNFPARNRAEDQTSPVVTTRNQYITDIAAEQLTYVLIRLRRFWAEThGFSR
YTIMSSASRDNLTSPGPLSAQNFRVTHVTITEVFLHWDPPDPVFFHHYLITILDVENQSKSIILRTLN
SLSLVLIGLKKYTKYKMRVAASTHVGESSLSEENDIFVRTSEDEPESSPQDVEVIDVTADEIRLKWSP
PEKPNGIIIAYEVLYKNIDTLYMKNTSTTDIILRNLRPHTLYNISVRSYTRFGHGNQVSSLLSVRTSE
SVPDSAPENITYKNISSGEIELSFLPPSSPNGIIQKYTIYLKRSNGNEERTINTTSLTQNIKGLKKYT
QYIIEVSASTLKGEGVRSAPISILTEEDAPDSPPQDFSVKQLSGVTVKLSWQPPLEPNGIILYYTVYV
WRSSLKTINVTETSLELSDLDYNVEYSAYVTASTRFGDGKTRSNIISFQTPEGPSDPPKDVYYANLSS
SSIILFWTPPSKPNGIIQYYSVYYRNTSGTFMQNFTLHEVTNDFDNMTVSTIIDKLTIFSYYTFWLTA
STSVGNGNKSSDIIEVYTDQDVPEGFVGNLTYESISSTAINVSWVPPAQPNGLVFYYVSLILQQTPRH
VRPPLVTYERSIYFDNLEKYTDYILKITPSTEKGFSDTYTAQLYIKTEEDIPETSPIINTFKNLSSTS
VLLSWDPPVKPNGAIISYDLTLQGPNENYSFITSDNYIILEELSPFTLYSFFAAARTRKGLGPSSILF
FYTDESVPLAPPQNLTLINCTSDFVWLKWSPSPLPGGIVKVYSFKIHEHETDTIYYKNISGFKTEAKL
VGLEPVSTYSIRVSAFTKVGNGMQFSNVVKFTTQESVPDVVQNMQCMATSWQSVLVKWDPPKKANGII
TQYMVTVERNSTKVSPQDHMYTFIKLLANTSYVFKVRASTSAGEGDESTCHVSTLPETVPSVPTNIAF
SDVQSTSATLTWIRPDTILGYFQNYKITTQLRAQKCKEWESEECVEYQKIQYLYEAHLTEETVYGLKK
FRWYRFQVAASTNAGYGNASNWISTKTLPGPPDGPPENVHVVATSPFSISISWSEPAVITGPTCYLID
VKSVDNDEFNISFIKSNEENKTIEIKDLEIFTRYSVVITAFTGNISAAYVEGKSSAEMIVTTLESAPK
DPPNNNTFQKIPDEVTKFQLTFLPPSQPNGNIQVYQALVYREDDPTAVQIHNLSIIQKTNTFVIANLE
GLKGGHTYNISVYAVNSAGAGPKVPMRITMDIKAPARPKTKPTPIYDATGKLLVTSTTITIRNPICYY
SDDHGPIKNVQVLVTETGAQHDGNVTKWYDAYFNKARPYFTNEGFPNPPCTEGKTKFSGNEEIYIIGA
DNACMIPGNEDKICNGPLKPKKQYLFKFRATNIMGQFTDSDYSDPVKTLGEGLSERTVEIILSVTLCI
LSIILLGTAIFAFARIRQKQKEGGTYSPQDAEIIDTKLKLDQLITVADLELKDERLTRPISKKSFLQH
VEELCTNNNLKFQEEFSELPKFLQDLSSTDADLPWNRAKNRFPNIKPYNNNRVKLIADASVPGSDYIN
ASYISGYLCPNEFIATQGPLPGTVGDFWRNVWETRAKTLVMLTQCFEKGRIRCHQYWPEDNKPVTVFG
DIVITKLMEDVQIDWTIRDLKIERHGDCMTVRQCNFTAWPEHGVPENSAPLIHFVKLVRASRAHDTTP
MIVHCSAGVGRTGVFIALDHLTQHINDHDFVDIYGLVAELRSERMCMVQNLAQYIFLHQCILDLLSNK
GSNQPICFVNYSAILQKMDSLDAMEGGDVELEWEETTM

| | | |
|---|---|---|
| NOV15b, CG50718-06 | SEQ ID NO: 167 | 6900 bp |

| | | |
|---|---|---|
| DNA Sequence | ORF Start: ATG at 1 | ORF Stop: TAA at 6898 |

ATGGATTTTCTTATCATTTTTCTTTTACTTTTTATTGGGACTTCAGAGACACAGGTAGATGTTTCCAA
TGTCGTTCCTGGTACTAGGTACGATATAACCATCTCTTCAATTTCTACAACATACACCTCACCTGTTA
CTAGAATAGTGACAACAAATGTAACAAAACCAGGGCCTCCAGTCTTCCTAGCCGGGGAAAGAGTCGGA
TCTGCTGGGATTCTTCTGTCTTGGAATACACCACCTAATCCAAATGGAAGGATTATATCTTACATTGT
CAAATATAAGGAAGTTTGTCCGTGGATGCAAACAGTATATACACAAGTCAGATCAAAGCCAGACAGTC
TGGAAGTTCTTCTTACTAATCTTAATCCTGGAACAACATATGAAATTAAGGTTGCTGCTGAAAACAGT
GCTGGCATTGGAGTGTTTAGTGATCCATTTCTCTTCCAAACTGCAGAAAGTGCTCCAGGAAAAGTGGT
GAATCTCACAGTTGAGGCCTACAACGCTTCAGCAGTTAAGCTGATTTGGTATTTACCTCGGCAACCAA
ATGGCAAAATTACCAGCTTCAAGATTAGTGTCAAGCATGCCAGAAGTGGGATAGTAGTGAAAGATGTC
TCAATCAGAGTAGAGGACATTTTGACTGGGAAATTGCCAGAATGCAATGAGAATAGTGAATCTTTTTT
ATGGAGTACAGCCAGCCCTTCTCCAACCCTTGGTAGAGTTACACCTCCATCGCGTACCACACATTCAT
CAAGCACGTTGACACAGAATGAGATCAGCTCTGTGTGGAAAGAGCCTATCAGTTTTGTAGTGACACAC
TTGAGACCTTATACAACATATCTTTTTGAAGTTTCAGCTGTTACAACTGAAGCAGGTTATATTGATAG
TACGATTGTCAGAACACCAGAATCAGTGCCTGAAGGACCACCACAAAACTGCGTAACAGGCAACATCA
CAGGAAAGTCCTTTTCAATTTTATGGGACCCACCAACTATAGTAACAGGGAAATTTAGTTATAGAGTT
GAATTATATGGACCATCAGGTCGCATTTTGGATAACAGCACAAAAGACCTCAAGTTTGCATTCACTAA
CCTAACACCATTTACAATGTATGATGTCTATATTTGCGGCTGAAACCAGTGCAGGGACTGGGCCAAGT
CAAATATTTCAGTATTCACTCCACCAGATGTTCCAGGGGCAGTGTTTGATTTACAACTTGCAGAGGTA
GAATCCACGCAAGTAAGAATTACTTGGAAGAAACCACGACAACCAAATGGAATTATTAACCAATACCG
AGTGAAAGTGCTAGTTCCAGAGACAGGAATAATTTTGGAAAATACTTTGCTCACTGGAAATAATGAGT
ATATAAATGACCCCATGGCTCCAGAAATTGTGAACATAGTAGAGCCAATGGTAGGATTATATGAGGGT
TCAGCAGAGATGTCGTCTGACCTTCACTCACTTGCTACATTTATATATAACAGCCATCCAGATAAAAA
CTTTCCTGCAAGGAATAGAGCTGAAGACCAGACTTCACCAGTTGTAACTACAAGGAATCAGTATATTA
CTGACATTGCAGCTGAACAGCTGTCTTATGTTATCAGGAGACTTGTACCTTTCACTGAGCACATGATT
AGTGTATCTGCTTTCACCATCATGGGAGAAGGACCACCAACAGTTCTCAGTGTTAGGACACGTCAGCA
AGTGCCAAGCTCCATTAAAATTATAAACTATAAAAATATTAGTTCTTCATCTATTTTGTTATATTGGG
ATCCTCCAGAATATCCCAATGGAAAAATAACTCACTATACGATTTATGCAATGGAATTGGATACAAAC
AGAGCATTCCAGATAACTACCATAGATAACAGCTTTCTCATAACAGGGTTAAAGAAATACACAAAATA
CAAAATGAGAGTGGCAGCCTCAACCCACGATGGAGAAAGTTCTTTGTCTGAAGAAAATGACATCTTTG
TGAGAACTTCAGAAGATGAACCGGAATCATCACCTCAAGATGTCGAAGTAATTGATGTTACCGCAGAT
GAAATAAGGTTGAAGTGGTCACCACCCGAAAAGCCCAATGGGATCATTATTGCTTATGAAGTGCTATA
TAAAAATATAGATACTTTATATATGAAGAACACATCAACAACAGACATAATATTAAGGAACTTAAGAC
CTCACACCCTCTATAACATTTCTGTAAGGTCTTACACCAGATTTGGTCATGGCAATCAGGTATCTTCT
TTACTCTCTGTAAGGACTTCGGAGACTGTGCCTGATAGTGCACCAGAAAATATCACTTACAAAAATAT
TTCTTCTGGAGAGATTGAGCTATCATTCCTTCCCCCAAGTAGTCCCAATGGAATCATAAAAAAATATA
CAATTTATCTCAAGAGAAGTAATGGAAATGAGGAAAGAACTATAAATACAACCTCTTTAACCCAAAAC
ATTAAAGTACTGAAGAAATATACCCAATATATCATTGAGGTGTCTGCTAGTACACTGAAAGGTGAAGG
AGTTCGGAGTGCTCCCATAAGTATACTGACGGAGGAAGATGCTCCTGATTCTCCCCCTCAAGACTTCT
CTGTAAAACAGTTGTCTGGTGTCACGGTGAAGTTGTCATGGCAACCACCCCTGGAGCCAAATGGAATT
ATCCTTTATTACACAGTTTATGTCTGGAATAGATCATCATTAAAAACTATTAATGTCACTGAAACATC
ATTGGAGTTATCAGATTTGGATTATAATGTTGAATACAGTGCTTATGTAACAGCTAGCACCAGATTTG
GTGATGGGAAAACAAGAAGCAATATCATTAGCTTTCAAACACCAGAGGGAGCACCAAGCGATCCTCCC
AAAGATGTTTATTATGCAAACCTCAGTTCTTCATCAATAATTCTTTTCTGGACACCTCCTTCAAAACC
TAATGGGATTATACAATATTACTCTGTTTATTACAGAAATACTTCAGGTACTTTTATGCAGAATTTTA
CACTCCATGAAGTAACCAATGACTTTGACAATATGACTGTATCCACAATTATAGATAAACTGACAATA
TTCAGCTACTATACATTTTGGTTAACAGCAAGTACTTCAGTTGGAAATGGGAATAAAAGCAGTGACAT
CATTGAAGTATACACAGATCAAGACGTCCCTGAAGGGTTTGTTGGAAACCTGACTTACGAATCCATTT
CGTCAACTGCAATAAATGTAAGCTGGGTCCCACCGGCTCAACCAAACGGTCTAGTCTTCTACTATGTT
TCACTGATCTTACAGCAGACTCCTCGCCATGTGAGACCACCTCTTGTTACATATGAGAGAAGCATATA
TTTTGATAATCTGGAAAAATACACTGATTATATATTAAAAATTACTCCATCAACAGAAAAGGGATTCT
CTGATACCTATACTGCCCAGCTATACATCAAGACTGAAGAAGATGTCCCAGAAACTTCACCAATAATC
AACACTTTTAAAAACCTTTCCTCTACCTCAGTTCTCTTATCATGGGATCCCCCAGTAAAGCCAAATGG
TGCAATAATAAGTTATGATTTAACTTTACAAGGACCAAATGAAAATTATTCTTTCATTACTTCTGATA
ATTACATAATATTGGAAGAGCTTTCACCATTTACATTATATAGCTTTTTTGCTGCCGCAAGAACTAGA
AAAGGACTTGGTCCTTCCAGTATTCTTTTCTTTTACACAGATGAGTCAGTGCCGTTAGCACCTCCACA
AAATTTGACTTTAATCAACTGTACTTCAGACTTTGTATGGCTGAAATGGAGCCCAAGTCCTCTTCCAG
GTGGTATTGTTAAAGTATATAGTTTTAAAATTCATGAACATGAAACTGACACTATATATTATAAGAAT
ATATCAGGATTTAAAACTGAAGCCAAACTTGTTGGACTGGAACCAGTCAGCACCTACTCTATCCGTGT
ATCTGCGTTCACCAAAGTTGGAAATGGCAATCAATTTAGTAATGTAGTAAAATTCACAACCCAAGAAT
CAGTTCCAGATGTCGTGCAGAATATGCAGTGCATGGCAACTAGCTGGCAGTCAGTTTTAGTGAAATGG
GATCCACCCAAAAAGGCAAATGGAATAATAACGCAGTATATGGTAACAGTTGAAAGGAATTCTACAAA
AGTTTCTCCCCAAGATCACATGTACACTTTCATAAAGCTTCTTGCCAATACCTCATATGTCTTTAAAG
TAAGAGCTTCAACCTCAGCTGGTGAAGGTGATGAAAGCACATGCCATGTCAGCACACTACCTGAAACA
GTTCCCAGTGTTCCCACAAATATTGCTTTTTCTGATGTTCAGTCAACTAGTGCAACATTGACATGGAT
AAGACCTGACACTATCCTTGGCTACTTTCAAAATTACAAAATTACCACTCAACTTCGTGCTCAAAAAT
GCAAAGAATGGGAATCCGAAGAATGTGTTGAATATCAAAAAATTCAATACCTCTATGAAGCTCACTTA
ACTGAAGAGACAGTATATGGATTAAAGAAATTTAGATGGTATAGATTCCAAGTGGCTGCCAGCACCAA
TGCTGGCTATGGCAATGCTTCAAACTGGATTTCTACAAAAACTCTGCCTGGCCCTCCAGATGGTCCTC
CTGAAAATGTTCATGTAGTAGCAACATCACCTTTTAGCATCAGCATAAGCTGGAGTGAACCTGCTGTC
ATTACTGGACCAACATGTTATCTGATTGATGTCAAATCGGTAGATAATGATGAATTTAATATATCCTT
CATCAAGTCAAATGAAGAAAATAAAACCATAGAAATTAAAGATTTAGAAATATTCACAAGGTATTCTG
TAGTGATCACTGCATTTACTGGGAACATTAGTGCTGCATATGTAGAAGGGAAGTCAAGTGCTGAAATG
ATTGTTACTACTTTAGAATCAGCCCCAAAGGACCCACCTAACAACATGACATTTCAGAAGATACCAGA
TGAAGTTACAAAATTTCAATTAACGTCCCTTCCTCCTTCTCAACCTAATGGAAATATCCAAGTATATC
AAGCTCTGGTTTACCGAGAAGATGATCCTACTGCTGTCCAGATTCACAACCTCAGTATTATACAGAAA
ACCAACACATTCGTCATTGCAATGCTAGAAGGACTAAAAGGTGGACATACATACAATATCAGTGTTTA
CGCAGTCAATAGTGCTGGTGCAGGTCCAAAGGTTCCGATGAGAATAACCATGGATATCAAAGCTCCAG
CACGACCAAAAACCAAACCAACCCCTATTTATGATGCCACAGGAAAACTGCTTGTGACTTCAACAACA
ATTACAATCAGAATGCCAATATGTTACTACAGTGATGATCATGGACCAATAAAAAATGTACAAGTGCT
TGTGACAGAAACAGGAGCTCAGCATGATGGAAATGTAACAAAGTGGTATGATGCATATTTTAATAAAG
CAAGGCCATATTTTACAAATGAAGGCTTTCCTAACCCTCCATGTACAGAAGGAAAGACAAAGTTTAGT
GGCAATGAAGAAATCTACATCATAGGTGCTGATAATGCATGCATGATTCCTGGCAATGAAGACAAAAT
TTGCAATGGACCACTGAAACCAAAAAAGCAATACTTATTTAAATTTAGAGCTACAAATATTATGGGAC
AATTTACTGACTCTGATTATTCTGACCCTGTTAAGACTTTAGGCGAAGGACTTTCAGAAAGAACCCTA
GAGATCATTCTTTCCGTCACTTTGTGTATCCTTTCAATAATTCTCCTTGGAACAGCTATTTTTGCATT
TGCAAGAATTCGACAGAAGCAGAAAGAAGGTGGCACATACTCTCCTCAGGATGCAGAAATTATTGACA
CTAAATTGAAGCTGGATCAGCTCATCACAGTGGCAGACCTGGAACTGAAGGACGAGAGATTAACGCGG
TTACTTAGTTATAGAAAATCCATCAAGCCAATAAGCAAGAAATCCTTCCTGCAACATGTTGAAGAGCT
TTGCACAAACAACAACCTAAAGTTTCAAAGAAGAATTTTCGGAATTACCAAATTTCTTCAGGATCTTT
CTTCAACTGATGCTGATCTGCCTTGGAATAGAGCAAAAAACCGCTTCCCAAACATAAAACCATATAAT
AATAACAGAGTAAAGCTGATAGCTGACGCTAGTGTTCCAGGTTCGGATTATATTAATGCCAGCTATAT
TTCTGGTTATTTATGTCCAAATGAATTTATTGCTACTCAAGGTCCACTACCAGGAACAGTTGGAGATT
TTTGGAGAATGGTGTGGGAAACCAGAGCAAAAACATTAGTAATGCTAACACAGTGTTTTGAAAAAGGA
CGGATCAGATGCCATCAGTATTGGCCAGAGGACAACAAGCCAGTTACTGTCTTTGGAGATATAGTGAT
TACAAAGCTAATGGAGGATGTTCAAATAGATTGGACTATCAGGGATCTGAAAATTGAAAGGCATGGGG
ATTGCATGACTGTTCGACAGTGTAACTTTACTGCCTGGCCAGAGCATGGGGTTCCTGAGAACAGCGCC
CCTCTAATTCACTTTGTGAAGTTGGTTCGAGCAAGCAGGGCACATGACACCACACCTATGATTGTTCA
CTGTAGTGCTGGAGTTGGAAGAACTGGAGTTTTTATTGCTCTGGACCATTTAACACAACATATAAATG
ACCATGATTTTGTGGATATATATGGACTAGTAGCTGAACTGAGAAGTGAAAGAATGTGCATGGTGCAG
AATCTGGCACAGTATATCTTTTTACACCAGTGCATTCTGGATCTCTTATCAAATAAGGGAAGTAATCA
GCCCATCTGTTTTGTTAACTATTCAGCACTTCAGAAGATGGACTCTTTGGACGCCATGGAAGGTGATG
TTGAGCTTGAATGGGAAAGAAACCACTATGTAA

NOV15b, CG50718-06

| | | | |
|---|---|---|---|
| Protein Sequence | SEQ ID NO: 168 | 2299 aa | MW at 257243.9kD |

MDFLIIFLLLFIGTSETQVDVSNVVPGTRYDITISSISTTYTSPVTRIVTTNVTKPGPPVFLAGERVG
SAGILLSWNTPPNPNGRIISYIVKYKEVCPWMQTVYTQVRSKPDSLEVLLTNLNPGTTYEIKVAAENS
AGIGVFSDPFLFQTAESAPGKVVNLTVEAYNASAVKLIWYLPRQPNGKITSFKISVKHARSGIVVKDV
SIRVEDILTGKLPECNENSESFLWSTASPSPTLGRVTPPSRTTHSSSTLTQNEISSVWKEPISFVVTH
LRPYTTYLFEVSAVTTEAGYIDSTIVRTPESVPEGPPQNCVTGNITGKSFSILWDPPTIVTGKFSYRV
ELYGPSGRILDNSTKDLKFAFTNLTPFTMYDVYIAAETSAGTGPKSNISVFTPPDVPGAVFDLQLAEV
ESTQVRITWKKPRQPNGIINQYRVKVLVPETGIILENTLLTGNNEYINDPMAPEIVNIVEPMVGLYEG
SAEMSSDLHSLATFIYNSHPDKNFPARNRAEDQTSPVVTTRNQYITDIAAEQLSYVIRRLVPFTEHMI
SVSAFTIMGEGPPTVLSVRTRQQVPSSIKIINYKNISSSSILLYWDPPEYPNGKITHYTIYAMELDTN
RAFQITTIDNSFLITGLKKYTKYKMRVAASTHDGESSLSEENDIFVRTSEDEPESSPQDVEVIDVTAD
EIRLKWSPPEKPNGIIIAYEVLYKNIDTLYMKNTSTTDIILRNLRPHTLYNISVRSYTRFGHGNQVSS
LLSVRTSETVPDSAPENITYKNISSGEIELSFLPPSSPNGIIKKYTIYLKRSNGNEERTINTTSLTQN
IKVLKKYTQYIIEVSASTLKGEGVRSAPISILTEEDAPDSPPQDFSVKQLSGVTVKLSWQPPLEPNGI
ILYYTVYVWNRSSLKTINVTETSLELSDLDYNVEYSAYVTASTRFGDGKTRSNIISFQTPEGAPSDPP
KDVYYANLSSSSIILFWTPPSKPNGIIQYYSVYYRNTSGTFMQNFTLHEVTNDFDNMTVSTIIDKLTI
FSYYTFWLTASTSVGNGNKSSDIIEVYTDQDVPEGFVGNLTYESISSTAINVSWVPPAQPNGLVFYYV
SLILQQTPRHVRPPLVTYERSIYFDNLEKYTDYILKITPSTEKGFSDTYTAQLYIKTEEDVPETSPII
NTFKNLSSTSVLLSWDPPVKPNGAIISYDLTLQGPNENYSFITSDNYIILEELSPFTLYSFFAAARTR
KGLGPPSSILFFYTDESVPLAPPQNLTLINCTSDFVWLKWSPSPLPGGIVKVYSFKIHEHTDTIYYKN
ISGFKTEAKLVGLEPVSTYSIRVSAFTKVGNGNQFSNVVKFTTQESVPDVVQNMQCMATSWQSVLVKW
DPPKKANGIITQYMVTVERNSTKVSPQDHMYTFIKLLANTSYVFKVRASTSAGEGDESTCHVSTLPET
VPSVPTNIAFSDVQSTSATLTWIRPDTILGYFQNYKITTQLRAQKCKEWESEECVEYQKIQYLYEAHL
TEETVYGLKKFRWYRFQVAASTNAGYGNASNWISTKTLPGPPDGPPENVHVVATSPFSISISWSEPAV
ITGPTCYLIDVKSVDNDEFNISFIKSNEENKTIEIKDLEIFTRYSVVITAFTGNISAAYVEGKSSAEM
IVTTLESAPKDPPNNMTFQKIPDEVTKFQLTSLPPSQPNGNIQVYQALVYREDDPTAVQIHNLSIIQK
TNTFVIAMLEGLKGGHTYNISVYAVMSAGAGPKVPMRITMDIKAPARPKTKPTPIYDATGKLLVTSTT
ITIRMPICYYSDDHGPIKNVQVLVTETGAQHDGNVTKWYDAYFNKARPYFTNEGFPNPPCTEGKTKFS
GNEEIYIIGADNACMIPGNEDKICNGPLKPKKQYLFKFRATNIMGQFTDSDYSDPVKTLGEGLSERTL
EIILSVTLCILSIILLGTAIFAFARIRQKQKEGGTYSPQDAEIIDTKLKLDQLITVADLELKDERLTR
LLSYRKSIKPISKKSFLQHVEELCTNNNLKFQEEFSELPKFLQDLSSTDADLPWNRAKNRFPNIKPYN
NNRVKLIADASVPGSDYINASYISGYLCPNEFIATQGPLPGTVGDFWRMVWETRAKTLVMLTQCFEKG
RIRCHQYWPEDNKPVTVFGDIVITKLMEDVQIDWTIRDLKIERHGDCMTVRQCNFTAWPEHGVPENSA
PLIHFVKLVRASRAHDTTPMIVHCSAGVGRTGVFIALDHLTQHINDHDFVDIYGLVAELRSERMCMVQ
NLAQYIFLHQCILDLLSNKGSNQPICFVNYSALQKMDSLDAMEGDVELEWEETTM

| | | |
|---|---|---|
| NOV15c, 258979883 | SEQ ID NO: 169 | 2220 bp |

| | | |
|---|---|---|
| DNA Sequence | ORF Start: at 1 | ORF Stop: end of sequence |

AGATCTCCTGAAGGACCACCACAAAACTGCGTAACAGGCAACATCACAGGAAAGTCCTTTTCAATTTT
ATGGGACCCACCAACTATAGTAACAGGGAAATTTAGTTATAGAGTTGAATTATATGGACCATCAGGTC
GCATTTTGGATAACAGCACAAAAGACCTCAAGTTTGCATTCACTAACCTAACACCATTTACAATGTAT
GATGTCTATATTGCGGCTGAAACCAGTGCAGGGACTGGGCCCAAGTCAAATATTTCAGTATTCACTCC
ACCAGATGTTCCAGGGGCAGTGTTTGATTTACAACTTGCAGAGGTAGAATCCACGCAAGTAAGAATTA
CTTGGAAGAAACCACGACAACCAAATGGAATTATTAACCAATACCGAGTGAAAGTGCTAGTTCCAGAG
ACAGGAATAATTTTGGAAAATACTTTGCTCACTGGAAATAATGAGTATATAAATGACCCCATGGCTCC
AGAAATTGTGAACATAGTAGAGCCAATGGTAGGATTATATGAGGGTTCAGCAGAGATGTCGTCTGACC
TTCACTCACTTGCTACATTTATATATAACAGCCATCCAGATAAAAACTTTCCTGCAAGGAATAGAGCT
GAAGACCAGACTTCACCAGTTGTAACTACAAGGAATCAGTATATTACTGACATTGCAGCTGAACAGCT
GTCTTATGTTATCAGGAGACTTGTACCTTTCACTGAGCACATGATTAGTGTATCTGCTTTCACCATCA
TGGGAGAAGGACCACCAACAGTTCTCAGTGTTAGGACACGTCAGCAAGTGCCAAGCTCCATTAAAATT
ATAAACTATAAAAATATTAGTTCTTCATCTATTTTGTTATATTGGGATCCTCCAGAATATCCCAATGG
AAAAATAACTCACTATACGATTTATGCAATGGAATTGGATACAAACAGAGCATTCCAGATAACTACCA
TAGATAACAGCTTTCTCATAACAGGGTTAAAGAAATACACAAAATACAAAATGAGAGTGGCAGCCTCA
ACCCACGATGGAGAAAGTTCTTTGTCTGAAGAAAATGACATCTTTGTGAGAACTTCAGAAGATGAACC
GGAATCATCACCTCAAGATGTCGAAGTAATTGATGTTACCGCAGATGAAATAAGGTTGAAGTGGTCAC
CACCCGAAAAGCCCAATGGGATCATTATTGCTTATGAAGTGCTATATAAAAATATAGATACTTTATAT
ATGAAGAACACATCAACAACAGACATAATATTAAGGAACTTAAGACCTCACACCCTCTATAACATTTC
TGTAAGGTCTTACACCAGATTTGGTCATGGCAATCAGGTATCTTCTTTACTCTCTGTAAGGACTTCGG
AGACTGTGCCTGATAGTGCACCAGAAAATATCACTTACAAAAATATTTCTTCTGGAGAGATTGAGCTA
TCATTCCTTCCCCCAAGTAGTCCCAATGGAATCATAAAAAAATATACAATTTATCTCAAGAGAAGTAA
TGGAAATGAGGAAAGAACTATAAATACAACCTCTTTAACCCAAACATTAAAAGTACTGAAGAAATATA
CCCAATATATCATTGAGGTGTCTGCTAGTACACTGAAAGGTGAAGGAGTTCGGAGTGCTCCCATAAGT
ATACTGACGGAGGAAGATGCTCCTGATTCTCCCCCTCAAGACTTCTCTGTAAAACAGTTGTCTGGTGT
CACGGTGAAGTTGTCATGGCAACCACCCCTGGAGCCAAATGGAATTATCCTTTATTACACAGTTTATG
TCTGGAATAGATCATCATTAAAAACTATTAATGTCACTGAAACATCATTGGAGTTATCAGATTTGGAT
TATAATGTTGAATACAGTGCTTATGTAACAGCTAGCACCAGATTTGGTGATGGGAAAACAAGAAGCAA
TATCATTAGCTTTCAAACACCAGAGGGAGCACCAAGCGATCCTCCCAAAGATGTTTATTATGCAAACC
TCAGTTCTTCATCAATAATTCTTTTCTGGACACCTCCTTCAAAACCTAATGGGATTATACAATATTAC
TCTGTTTATTACAGAAATACTTCAGGTACTTTTATGCAGAATTTTACACTCCATGAAGTAACCAATGA
CTTTGACAATATGACTGTATCCACAATTATAGATAAACTGACAATATTCAGCTACTATACATTTTGGT
TAACAGCAAGTACTTCAGTTGGAAATGGGAATAAAAGCCTCGAG

NOV15c, 258979883

| | | | |
|---|---|---|---|
| Protein Sequence | SEQ ID NO: 170 | 740 aa | MW at 82862.3kD |

RSPEGPPQNCVTGNITGKSFSILWDPPTIVTGKFSYRVELYGPSGRILDNSTKDLKFAFTNLTPFTMY
DVYIAAETSAGTGPKSNISVFTPPDVPGAVFDLQLAEVESTQVRITWKKPRQPNGIINQYRVKVLVPE
TGIILENTLLTGNNEYINDPMAPEIVNIVEPMVGLYEGSAEMSSDLHSLATFIYNSHPDKNFPARNRA
EDQTSPVVTTRNQYITDIAAEQLSYVIRRLVPFTEHMISVSAFTIMGEGPPTVLSVRTRQQVPSSIKI
INYKNISSSSILLYWDPPEYPNGKITHYTIYA4ELDTNRAFQITTIDNSFLITGLKKYTKYKMRVAAS
THDGESSLSEENDIFVRTSEDEPESSPQDVEVIDVTADEIRLKWSPPEKPNGIIIAYEVLYKNIDTLY
MKNTSTTDIILRNLRPHTLYNISVRSYTRFGHGNQVSSLLSVRTSETVPDSAPENITYKNISSGEIEL
SFLPPSSPNGIIKKYTIYLKRSNGNEERTINTTSLTQNIKVLKKYTQYIIEVSASTLKGEGVRSAPIS
ILTEEDAPDSPPQDFSVKQLSGVTVKLSWQPPLEPNGIILYYTVYVWNRSSLKTINVTETSLELSDLD
YNVEYSAYVTASTRFGDGKTRSNIISFQTPEGAPSDPPKDVYYANLSSSSIILFWTPPSKPNGIIQYY
SVYYRNTSGTFMQNFTLHEVTNDFDNMTVSTIIDKLTIFSYYTFWLTASTSVGNGNKSLE

| | | |
|---|---|---|
| NOV5d, CG50718-01 | SEQ ID NO: 171 | 6994 bp |

| | | |
|---|---|---|
| DNA Sequence | ORF Start: ATG at 31 | ORF Stop: TAA at 6874 |

TGATTCTACTGGCTGAAAAATGTAATAAAGATGGATTTTCTTATCATTTTTCTTTTACTTTTTATTGG
GACTTCAGAGACACAGGTAGATGTTTCCAATGTCGTTCCTGGTACTAGGTACGATATAACCATCTCTT
CAATTTCTACAACATACACCTCACCTGTTACTAGAATAGGGGCTTCTAATGAACCAGGGCCTCCAGTC
TTCCTAGCCGGGGAAAGAGTCGGATCTGCTGGGATTCTTCTGTCTTGGAATACACCACCTAATCCAAA
TGGAAGGATTATATCTTACATTGTCAAATATAAGGAAGTTTGTCCGTGGATGCAAACAGTATATACAC
AAGTCAGATCAAAGCCAGACAGTCTGGAAGTTCTTCTTACTAATCTTAATCCTGGAACAACATATGAA
ATTAAGGTAGCTGCTGAAAACAGTGCTGGCATTGGAGTGTTTAGTGATCCATTTCTCTTCCAAACTGC
AGAAAGTGCTCCAGGAAAAGTGGTGGATTTCACAGGTGAGGCTGTCCCGTTCAGCAGTAAGCTGATGT
GGTATACCTCGGCAACCAAAAAAAAAATTACCAGCTTCAAGATTAGTGTCAAGCATAACAGAAGTGGG
ATAGTAGTGAAAGAAGTGTCAATCAGAGTGGAGTGCATTTTAAGTGCTTCCCTTCCTTTGCACTGCAA
CGAGAATAGTGAATCTTTTTTATGGAGTACAGCCAGCCCTTCTCCAACCCTTGGTAGAGTTACACCTC
CATCGCGTACCACACATTCATCAAGCACGTTGACACAGAATGAGATCAGCTCTGTGAAAGAGCCTATC
AGTTTTGTAGTGACACACTTGAGACCTTATACAACATATCTTTTTGAAGTTTCAGCTGCTACAACTGA
AGCAGGTTATATTGATAGTACGATTGTCAGAACACCAGAATCAGTGCCTGAAGGACCACCACAAAACT
GCGTAACAGGCAACATCACAGGAAAGTCCTTTTCAATTTTATGGGACCCACCAACTATAGTAACAGGG
AAATTTAGTTATAGAGTTGAATTATATGGACCATCAGCAGGTCGCATTTTGGATAACAGCACAAAAGA
CCTCAAGTTTGCATTCACTAACCTAACACCATTTACAATGTATGATGTCTATATTGCGGCTGAAACCA
GTGCAGGGACTGGGCCCAAGTCAAATATTTCAGTATTCACTCCACCAGATGTTCCAGGGGCAGTGTTT
GATTTACAACTTGCAGAGGTAGAATCCACGCAAGTAAGAATTACTTGGAAGAAACCACGACAACCAAA
TGGAATTATTAACCAATACCGAGTGAAAGTGCTAGTTCCAGAGACAGGAATAATTTTGGAAAATACTT
TGCTCACTGGAAATAATGAGATAAATGACCCCATGGCTCCAGAAATTGTGAACATAGTACAGCCAATG
GTAGGATTATATGAGGGTTCAGCAGAGATGTCGTCTGACCTTCACTCACTTGCTACATTTATATATAA
CAGCCATCCAGATAAAAACTTTCCTGCAAGGAATAGAGCTGAAGACCAGACTTCACCAGTTGTAACTA
CAAGGAATCAGTATATTACTGACATTGCAGCTGAACAGCTGACTTATGTTCTTATCAGATTAAGGAGA
TTTTGGGCTGAGACAATGGGGTTTTCTAGATATACAATCATGTCATCTGCAAGCAGGGACAATTTGAC
TTCCCCAGGCCCTTTGTCAGCCCAAAATTTCAGAGTTACACATGTTACCATAACAGAAGTATTTTTAC
ACTGGGATCCTCCAGATCCTGTATTTTTTCATCATTACCTTATCACTATTTTGGATGTTGAAAACCAA
TCCAAGAGTATTATTTTAAGGACATTAAACAGTTTGTCTCTTGTCCTTATAGGGTTAAAGAAATACAC
AAAATACAAAATGAGAGTGGCAGCCTCAACCCACGTTGGAGAAAGTTCTTTGTCTGAAGAAAATGACA
TCTTTGTGAGAACTTCAGAAGATGAACCGGAATCATCACCTCAAGATGTCGAAGTAATTGATGTTACC
GCAGATGAAATAAGGTTGAAGTGGTCACCACCCGAAAAGCCCAATGGGATCATTATTGCTTATGAAGT
GCTATATAAAAATATAGATACTTTATATATGAAGAACACATCAACAACAGACATAATATTAAGGAACT
TAAGACCTCACACCCTCTATAACATTTCTGTAAGGTCTTACACCAGATTTGGTCATGGCAATCAGGTA
TCTTCTTTACTCTCTGTAAGGACTTCGGAGTCAGTGCCTGATAGTGCACCAGAAAATATCACTTACAA
AAATATTTCTTCTGGAGAGATTGAGCTATCATTCCTTCCCCCAAGTAGTCCCAATGGAATCATACAAA
AATATACAATTTATCTCAAGAGAAGTAATGGAAATGAGGAAAGAACTATAAATACAACCTCTTTAACC
CAAAACATTAAAGGTCTGAAGAAATATACCCAATATATCATTGAGGTGTCTGCTAGTACACTCAAAGG
TGAAGGAGTTCGGAGTGCTCCCATAAGTATACTGACGGAGGAAGATGCTCCTGATTCTCCCCCTCAAG
ACTTCTCTGTAAAACAGTTGTCTGGTGTCACGGTGAAGTTGTCATGGCAACCACCCCTGGAGCCAAAT
GGAATTATCCTTTATTACACAGTTTATGTCTGGAGATCATCATTAAAAACTATTAATGTCACTGAAAC
ATCATTGGAGTTATCAGATTTGGATTATAATGTTGAATACAGTGCTTATGTAACAGCTAGCACCAGAT
TTGGTGATGGGAAAACAAGAAGCAATATCATTAGCTTTCAAACACCAGAGGGACCAAGCGATCCTCCC
AAAGATGTTTATTATGCAAACCTCAGTTCTTCATCAATAATTCTTTTCTGGACACCTCCTTCAAAACC
TAATGGGATTATACAATATTACTCTGTTTATTACAGAAATACTTCAGGTACTTTTATGCAGAATTTTA
CACTCCATGAAGTAACCAATGACTTTGACAATATGACTGTATCCACAATTATAGATAAACTGACAATA
TTCAGCTACTATACATTTTGGTTAACAGCAAGTACTTCAGTTGGAAATGGGAATAAAAGCAGTGACAT
CATTGAAGTATACACAGATCAAGACGTACCTGAAGGGTTTGTTGGAAACCTGACTTACGAATCCATTT
CGTCAACTGCAATAAATGTAAGCTGGGTCCCACCGGCTCAACCAAACGGTCTAGTCTTCTACTATGTT
TCACTGATCTTACAGCAGACTCCTCGCCATGTGAGACCACCTCTTGTTACATATGAGAGAAGCATATA
TTTTGATAATCTGGAAAAATACACTGATTATATATTAAAAATTACTCCATCAACAGAAAAGGGATTCT
CTGATACCTATACTGCCCAGCTATACATCAAGACTGAAGAAGATATCCCAGAAACTTCACCAATAATC
AACACTTTTAAAAACCTTTCCTCTACCTCAGTTCTCTTATCATGGGATCCCCCAGTAAAGCCAAATGG
TGCAATAATAAGTTATGATTTAACTTTACAAGGACCAAATGAAAATTATTCTTTCATTACTTCTGATA
ATTACATAATATTGGAAGAGCTTTCACCATTTACATTATATAGCTTTTTTGCTGCCGCAAGAACTAGA
AAAGGACTTGGTCCTTCCAGTATTCTTTTCTTTTACACAGATGAGTCAGTGCCGTTAGCACCTCCACA
AAATTTGACTTTAATCAACTGTACTTCAGACTTTGTATGGCTGAAATGGAGCCCAAGTCCTCTTCCAG
GTGGTATTGTTAAAGTATATAGTTTTAAAATTCATGAACATGAAACTGACACTATATATTATAAGAAT
ATATCAGGATTTAAAACTGAAGCCAAACTTGTTGGACTGGAACCAGTCAGCACCTACTCTATCCGTGT
ATCTGCGTTCACCAAAGTTGGAAATGGCAATCAATTTAGTAATGTAGTAAAATTCACAACCCAAGAAT
CAGTTCCAGATGTCGTGCAGAATATGCAGTGCATGGCAACTAGCTGGCAGTCAGTTTTAGTGAAATGG
GATCCACCCAAAAAGGCAAATGGAATAATAACGCAGTATATGGTAACAGTTGAAAGGAATTCTACAAA
AGTTTCTCCCCAAGATCACATGTACACTTTCATAAAGCTTCTTGCCAATACCTCATATGTCTTTAAAG
TAAGAGCTTCAACCTCAGCTGGTGAAGGTGATGAAAGCACATGCCATGTCAGCACACTACCTGAAACA
GTTCCCAGTGTTCCCACAAATATTGCTTTTTCTGATGTTCAGTCAACTAGTGCAACATTGACATGGAT
AAGACCTGACACTATCCTTGGCTACTTTCAAAATTACAAAATTACCACTCAACTTCGTGCTCAAAAAT
GCAAAGAATGGGAATCCGAAGAATGTGTTGAATATCAAAAAATTCAATACCTCTATGAAGCTCACTTA
ACTGAAGAGACAGTATATGGATTAAAGAAATTTAGATGGTATAGATTCCAAGTGGCTGCCAGCACCAA
TGCTGGCTATGGCAATGCTTCAAACTGGATTTCTACAAAAACTCTGCCTGGCCCTCCAGATGGTCCTC
CTGAAAATGTTCATGTAGTAGCAACATCACCTTTTAGCATCAGCATAAGCTGGAGTGAACCTGCTGTC
ATTACTGGACCAACATGTTATCTGATTGATGTCAAATCGGTAGATAATGATGAATTTAATATATCCTT
CATCAAGTCAAATGAAGAAAATAAAACCATAGAAATTAAAGATTTAGAAATATTCACAAGGTATTCTG
TAGTGATCACTGCATTTACTGGGAACATTAGTGCTGCATATGTAGAAGGGAAGTCAAGTGCTGAAATG
ATTGTTACTACTTTAGAATCAGCCCCAAAGGACCCACCTAACAACATGACATTTCAGAAGATACCAGA
TGAAGTTACAAAATTTCAATTAACGTTCCTTCCTCCTTCTCAACCTAATGGAAATATCCAAGTATATC
AAGCTCTGGTTTACCGAGAAGATGATCCTACTGCTGTCCAGATTCACAACCTCAGTATTATACAGAAA
ACCAACACATTCGTCATTGCAATGCTAGAAGGACTAAAAGGTGGACATACATACAATATCAGTGTTTA
CGCAGTCAATAGTGCTGGTGCAGGTCCAAAGGTTCCGATGAGAATAACCATGGATATCAAAGCTCCAG
CACGACCAAAAACCAAACCAACCCCTATTTATGATGCCACAGGAAAACTGCTTGTGACTTCAACAACA
ATTACAATCAGAATGCCAATATGTTACTACAGTGATGATCATGGACCAATAAAAAATGTACAAGTGCT
TGTGACAGAAACAGGAGCTCAGCATGATGGAAATGTAACAAAGTGGTATGATGCATATTTTAATAAAG
CAAGGCCATATTTTACAAATGAAGGCTTTCCTAACCCTCCATGTACAGAAGGAAAGACAAAGTTTAGT
GGCAATGAAGAAATCTACATCATAGGTGCTGATAATGCATGCATGATTCCTGGCAATGAAGACAAAAT
TTGCAATGGACCACTGAAACCAAAAAAGCAATACTTATTTAAATTTAGAGCTACAAATATTATGGGAC
AATTTACTGACTCTGATTATTCTGACCCTGTTAAGACTTTAGGCGAAGGACTTTCAGAAAGAACCGTA
GAGATCATTCTTTCCGTCACTTTGTGTATCCTTTCAATAATTCTCCTTGGAACAGCTATTTTTGCATT
TGCAAGAATTCGACAGAAGCAGAAAGAAGGTGGCACATACTCTCCTCAGGATGCAGAAATTATTGACA
CTAAATTGAAGCTGGATCAGCTCATCACAGTGGCAGACCTGGAACTGAAGGACGAGAGATTAACGCGG
CCAATAAGCAAGAAATCCTTCCTGCAACATGTTGAAGAGCTTTGCACAAACAACAACCTAAAGTTTCA
AGAAGAATTTTCGGAATTACCAAAATTTCTTCAGGATCTTTCTTCAACTGATGCTGATCTGCCTTGGA
ATAGAGCAAAAAACCGCTTCCCAAACATAAAACCATATAATAATAACAGAGTAAAGCTGATAGCTGAC
GCTAGTGTTCCAGGTTCGGATTATATTAATGCCAGCTATATTTCTGGTTATTTATGTCCAAATGAATT
TATTGCTACTCAAGGTCCACTACCAGGAACAGTTGGAGATITTTGGAGAATGGTGTGGGAAACCAGAG
CAAAAACATTAGTAATGCTAACACAGTGTTTTGAAAAAGGACGGATCAGATGCCATCAGTATTGGCCA
GAGGACAACAAGCCAGTTACTGTCTTTGGAGATATAGTGATTACAAAGCTAATGGAGGATGTTCAAAT
AGATTGGACTATCAGGGATCTGAAAATTGAAAGGCATGGGGATTGCATGACTGTTCGACAGTGTAACT
TTACTGCCTGGCCAGAGCATGGGGTTCCTGAGAACAGCGCCCCTCTAATTCACTTTGTGAAGTTGGTT
CGAGCAAGCAGGGCACATGACACCACACCTATGATTGTTCACTGCAGTGCTGGAGTTGGAAGAACTGG
AGTTTTTATTGCTCTGGACCATTTAACACAACATATAAATGACCATGATTTTGTGGATATATATGGAC
TAGTAGCTGAACTGAGAAGTGAAAGAATGTGCATGGTGCAGAATCTGGCACAGTATATCTTTTTACAC
CAGTGCATTCTGGATCTCTTATCAAATAAGGGAAGTAATCAGCCCATCTGTTTTGTTAACTATTCAGC
ACTTCAGAAGATGGACTCTTTGGACGCCATGGAAGGTGGTGATGTTGAGCTTGAATGGGAAGAAACCA
CTATGTAAATATTCAGACCAAAGGATACAATTGGAAGAGATTTTTAAATCCCAGGGGCCAAAGTTACC
CCCTCATTCTTCCGAATTGAAATGTGCAACCTTAAAGAAATATCTATGCTTCTCTCAC

NOV15d, CG50718-01

| | | | |
|---|---|---|---|
| Protein Sequence | SEQ ID NO: 172 | 2281 aa | MW at 255030.3kD |

MDFLIIFLLLFIGTSETQVDVSNVVPGTRYDITISSISTTYTSPVTRIGASNEPGPPVFLAGERVGSA
GILLSWNTPPNPNGRIISYIVKYKEVCPWMQTVYTQVRSKPDSLEVLLTNLNPGTTYEIKVAAENSAG
IGVFSDPFLFQTAESAPGKVVDFTGEAVPFSSKLMWYTSATKKKITSFKISVKHNRSGIVVKEVSIRV
ECILSASLPLHCNENSESFLWSTASPSPTLGRVTPPSRTTHSSSTLTQNEISSVKEPISFVVTHLRPY
TTYLFEVSAATTEAGYIDSTIVRTPESVPEGPPQNCVTGNITGKSFSILWDPPTIVTGKFSYRVELYG
PSAGRILDNSTKDLKFAFTNLTPFTMYDVYIAAETSAGTGPKSNISVFTPPDVPGAVFDLQLAEVEST
QVRITWKKPRQPNGIINQYRVKVLVPETGIILENTLLTGNNEINDPMAPEIVNIVQPMVGLYEGSAEM
SSDLHSLATFIYNSHPDKNFPARNRAEDQTSPVVTTRNQYITDIAAEQLTYVLIRLRRFWAETMGFSR
YTIMSSASRDNLTSPGPLSAQMFRVTHVTITEVFLHWDPPDPVFFHHYLITILDVENQSKSIILRTLN
SLSLVLIGLKKYTKYKMRVAASTHVGESSLSEENDIFVRTSEDEPESSPQDVEVIDVTADEIRLKWSP
PEKPNGIIIAYEVLYKNIDTLYMKNTSTTDIILRNLRPHTLYNISVRSYTRFGNGNQVSSLLSVRTSE
SVPDSAPENITYKNISSGEIELSFLPPSSPNGIIQKYTIYLKRSNGNEERTINTTSLTQNIKGLKKYT
QYIIEVSASTLKGEGVRSAPISILTEEDAPDSPPQDFSVKQLSGVTVKLSWQPPLEPNGIILYYTVYV
WRSSLKTINVTETSLELSDLDYNVEYSAYVTASTRFGDGKTRSNIISFQTPEGPSDPPKDVYYANLSS
SSIILFWTPPSKPNGIIQYYSVYYRNTSGTFMQNFTLHEVTNDFDNMTVSTIIDKLTIFSYYTFWLTA
STSVGNGNKSSDIIEVYTDQDVPEGFVGNLTYESISSTAINVSWVPPAQPNGLVFYYVSLILQQTPRH
VRPPLVTYERSIYFDNLEKYTDYILKITPSTEKGFSDTYTAQLYIKTEEDIPETSPIINTFKNLSSTS
VLLSWDPPVKPNGAIISYDLTLQGPNENYSFITSDNYIILEELSPFTLYSFFAAARTRKGLGPSSILF
FYTDESVPLAPPQNLTLINCTSDFVWLKWSPSPLPGGIVKVYSFKIHEHETDTIYYKNISGFKTEAKL
VGLEPVSTYSIRVSAFTKVGNGNQFSNVVKFTTQESVPDVVQNMQCMATSWQSVLVKWDPPKKANGII
TQYMVTVERNSTKVSPQDHMYTFIKLLANTSYVFKVRASTSAGEGDESTCHVSTLPETVPSVPTNIAF
SDVQSTSATLTWIRPDTILGYFQNYKITTQLRAQKCKEWESEECVEYQKIQYLYEAHLTEETVYGLKK
FRWYRFQVAASTNAGYGNASNWISTKTLPGPPDGPPENVHVVATSPFSISISWSEPAVITGPTCYLID
VKSVDNDEFNISF1KSNEENKTIEIKDLEIFTRYSVVITAFTGNISAAYVEGKSSAEMIVTTLESAPK
DPPNNMTFQKIPDEVTKFQLTFLPPSQPNGNIQVYQALVYREDDPTAVQIHNLSIIQKTNTFVIAMLE
GLKGGHTYNISVYAVNSAGAGPKVPMRITMDIKAPARPKTKPTPIYDATGKLLVTSTTITIRMPICYY
SDDHGPIKNVQVLVTETGAQHDGNVTKWYDAYFNKARPYFTNEGFPNPPCTEGKTKFSGNEEIYIIGA
DNACMIPGNEDKICNGPLKPKKQYLFKFRATNIMGQFTDSDYSDPVKTLGEGLSERTVEIILSVTLCI
LSIILLGTAIFAFARIRQKQKEGGTYSPQDAEIIDTKLKLDQLITVADLELKDERLTRPISKKSFLQH
VEELCTNNNLKFQEEFSELPKFLQDLSSTDADLPWNRAKNRFPNIKPYNNNRVKLIADASVPGSDYIN
ASYISGYLCPNEFIATQGPLPGTVGDFWRMVWETRAKTLVMLTQCFEKGRIRCHQYWPEDNKPVTVFG
DIVITKLMEDVOIDWTIRDLKIERHGDCMTVROCNFTAWPEHGVPENSAPLIHFVKLVRASRAHDTTP
MIVHCSAGVGRTGVFIALDHLTQHINDHDFVDIYGLVAELRSERNGMVQNLAQYIFLHQCILDLLSNK
GSNQPICFVNYSALQKMDSLDAMEGGDVELEWEETTM

| | | |
|---|---|---|
| NOV15e, CG50718-03 | SEQ ID NO: 173 | 2739 bp |

| | | |
|---|---|---|
| DNA Sequence | ORF Start: at 7 | ORF Stop: at 2734 |

AGATCTCAGGTAGATGTTTCCAATGTCGTTCCTGGTACTAGGTACGATATAACCATCTCTTCAATTTC
TACAACATACACCTCACCTGTTACTAGAATAGTGACAACAAATGTAACAAAACCAGGGCCTCCAGTCT
TCCTAGCCGGGGAAAGAGTCGGATCTGCTGGGATTCTTCTGTCTTGGAATACACCACCTAATCCAAAT
GGAAGGATTATATCTTACATTGTCAAATATAAGGAAGTTTGTCCGTGGATGCAAACAGTATATACACA
AGTCAGATCAAAGCCAGACAGTCTGGAAGTTCTTCTTACTAATCTTAATCCTGGAACAACATATGAAA
TTAAGGTTGCTGCTGAAAACAGTGCTGGCATTGGAGTGTTTAGTGATCCATTTCTCTTCCAAACTGCA
GAAAGTGCTCCAGGAAAAGTGGTGAATCTCACAGTTGAGGCCTACAACGCTTCAGCAGTTAAGCTGAT
TTGGTATTTACCTCGGCAACCAAATGGCAAAATTACCAGCTTCAAGATTAGTGTCAAGCATGCCAGAA
GTGGGATAGTAGTGAAAGATGTCTCAATCAGAGTAGAGGACATTTTGACTGGGAAATTGCCAGAATGC
AATGAGAATAGTGAATCTTTTTTATGGAGTACAGCCAGCCCTTCTCCAACCCTTGGTAGAGTTACACC
TCCATCGCGTACCACACATTCATCAAGCACGTTGACACAGAATGAGATCAGCTCTGTGTGGAAAGAGC
CTATCAGTTTTGTAGTGACACACTTGAGACCTTATACAACATATCTTTTTGAAGTTTCAGCTGTTACA
ACTGAAGCAGGTTATATTGATAGTACGATTGTCAGAACACCAGAATCAGTGCCTGAAGGACCACCACA
AAACTGCGTAACAGGCAACATCACAGGAAAGTCCTTTTCAATTTTATGGGACCCACCAACTATAGTAA
CAGGGAAATTTAGTTATAGAGTTGAATTATATGGACCATCAGGTCGCATTTTGGATAACAGCACAAAA
GACCTCAAGTTTGCATTCACTAACCTAACACCATTTACAATGTATGATGTCTATATTGCGGCTGAAAC
CAGTGCAGGGACTGGGCCCAAGTCAAATATTTCAGTATTCACTCCACCAGATGTTCCAGGGGCAGTGT
TTGATTTACAACTTGCAGAGGTAGAATCCACGCAAGTAAGAATTACTTGGAAGAAACCACGACAACCA
AATGGAATTATTAACCAATACCGAGTGAAAGTGCTAGTTCCAGAGACAGGAATAATTTTGGAAAATAC
TTTGCTCACTGGAAATAATGAGTATATAAATGACCCCATGGCTCCAGAAATTGTGAACATAGTAGAGC
CAATGGTAGGATTATATGAGGGTTCAGCAGAGATGTCGTCTGACCTTCACTCACTTGCTACATTTATA
TATAACAGCCATCCAGATAAAAACTTTCCTGCAAGGAATAGAGCTGAAGACCAGACTTCACCAGTTGT
AACTACAAGGAATCAGTATATTACTGACATTGCAGCTGAACAGCTGTCTTATGTTATCAGGAGACTTG
TACCTTTCACTGAGCACATGATTAGTGTATCTGCTTTCACCATCATGGGAGAAGGACCACCAACAGTT
CTCAGTGTTAGGACACGTCAGCAAGTGCCAAGCTCCATTAAAATTATAAACTATAAAAATATTAGTTC
TTCATCTATTTTGTTATATTGGGATCCTCCAGAATATCCCAATGGAAAAATAACTCACTATACGATTT
ATGCAATGGAATTGGATACAAACAGAGCATTCCAGATAACTACCATAGATAACAGCTTTCTCATAACA
GGGTTAAAGAAATACACAAAATACAAAATGAGAGTGGCAGCCTCAACCCACGATGGAGAAAGTTCTTT
GTCTGAAGAAAATGACATCTTTGTGAGAACTTCAGAAGATGAACCGGAATCATCACCTCAAGATGTCG
AAGTAATTGATGTTACCGCAGATGAAATAAGGTTGAAGTGGTCACCACCCGAAAAGCCCAATGGGATC
ATTATTGCTTATGAAGTGCTATATAAAAATATAGATACTTTATATATGAAGAACACATCAACAACAGA
CATAATATTAAGGAACTTAAGACCTCACACCCTCTATAACATTTCTGTAAGGTCTTACACCAGATTTG
GTCATGGCAATCAGGTATCTTCTTTACTCTCTGTAAGGACTTCGGAGACTGTGCCTGATAGTGCACCA
GAAAATATCACTTACAAAAATATTTCTTCTGGAGAGATTGAGCTATCATTCCTTCCCCCAAGTAGTCC
CAATGGAATCATACAAAAATATACAATTTATCTCAAGAGAAGTAATGGAAATGAGGAAAGAACTATAA
ATACAACCTCTTTAACCCAAAACATTAAAGGACTGAAGAAATATACCCAATATATCATTGAGGTGTCT
GCTAGTACACTCAAAGGTGAAGGAGTTCGGAGTGCTCCCATAAGTATACTGACGGAGGAAGATGCTCC
TGATTCTCCCCCTCAAGACTTCTCTGTAAAACAGTTGTCTGGTGTCACGGTGAAGTTGTCATGGCAAC
CACCCCTGGAGCCAAATGGAATTATCCTTTATTACACAGTTTATGTCTGGAATAGATCATCATTAAAA
ACTATTAATGTCACTGAAACATCATTGGAGTTATCAGATTTGGATTATAATGTTGAATACAGTGCTTA
TGTAACAGCTAGCCTCGAG

NOV15e, CG50718-03

| | | | |
|---|---|---|---|
| Protein Sequence | SEQ ID NO: 174 | 909 aa | MW at 100812.8kD |

QVDVSNVVPGTRYDITISSISTTYTSPVTRIVTTNVTKPGPPVFLAGERVGSAGILLSWNTPPNPNGR
IISYIVKYKEVCPWMQTVYTQVRSKPDSLEVLLTNLNPGTTYEIKVAAENSAGIGVFSDPFLFQTAES
APGKVVNLTVEAYNASAVKLIWYLPRQPNGKITSFKISVKHARSGIVVKDVSIRVEDILTGKLPECNE
NSESFLWSTASPSPTLGRVTPPSRTThSSSTLTQNEISSVWKEPISFVVTHLRPYTTYLFEVSAVTTE
AGYIDSTIVRTPESVPEGPPQNCVTGNITGKSFSILWDPPTIVTGKFSYRVELYGPSGRILDNSTKDL
KFAFTNLTPFTMYDVYIAAETSAGTGPKSNISVFTPPDVPGAVFDLQLAEVESTQVRITWKKPRQPNG
IINQYRVKVLVPETGIILENTLLTGNNEYINDPMAPEIVNIVEPMVGLYEGSAEMSSDLHSLATFIYN
SHPDKNFPARNRAEDQTSPVVTTRNQYITDIAAEQLSYVIRRLVPFTEHMISVSAFTIMGEGPPTVLS
VRTRQQVPSSIKIINYKRNSSSSILLYWDPPEYPNGKITHYTIYAMELDTNRAFQITTIDNSFLITGL
KKYTKYKMRVAASTHDGESSLSEENDIFVRTSEDEPESSPQDVEVIDVTADEIRLKWSPPEKPNGIII
AYEVLYKNIDTLYMKNTSTTDIILRNLRPHTLYNISVRSYTRFGHGNQVSSLLSVRTSETVPDSAPEN
ITYKNISSGEIELSFLPPSSPNGIIQKYTIYLKRSNGNEERTINTTSLTQNIKGLKKYTQYIIEVSAS
TLKGEGVRSAPISILTEEDAPDSPPQDFSVKQLSGVTVKLSWQPPLEPNGIELYYTVYVWNRSSLKTI
NVTETSLELSDLDYNVEYSAYVTAS

| | | |
|---|---|---|
| NOV15f, CG50718-04 | SEQ ID NO: 175 | 2565 bp |

| | | |
|---|---|---|
| DNA Sequence | ORF Start: at 7 | ORF Stop: at 2560 |

AGATCTCCTGAAGGGTTTGTTGGAAACCTGACTTACGAATCCATTTCGTCAACTGCAATAAATGTAAG
CTGGGTCCCACCGGCTCAACCAAACGGTCTAGTCTTCTACTATGTTTCACTGATCTTACAGCAGACTC
CTCGCCATGTGAGACCACCTCTTGTTACATATGAGAGAAGCATATATTTTGATAATCTGGAAAAATAC
ACTGATTATATATTAAAAATTACTCCATCAACAGAAAAGGGATTCTCTGATACCTATACTGCCCAGCT
ATACATCAAGACTGAAGAAGATGTCCCAGAAACTTCACCAATAATCAACACTTTTAAAAACCTTTCCT
CTACCTCAGTTCTCTTATCATGGGATCCCCCAGTAAAGCCAAATGGTGCAATAATAAGTTATGATTTA
ACTTTACAAGGACCAAATGAAAATTATTCTTTCATTACTTCTGATAATTACATAATATTGGAAGAGCT
TTCACCATTTACATTATATAGCTTTTTTGCTGCCGCAAGAACTAGAAAAGGACTTGGTCCTTCCAGTA
TTCTTTTCTTTTACACAGATGAGTCAGTGCCGTTAGCACCTCCACAAAATTTGACTTTAATCAACTGT
ACTTCAGACTTTGTATGGCTGAAATGGAGCCCAAGTCCTCTTCCAGGTGGTATTGTTAAAGTATATAG
TTrTAAAATTCATGAACATGAAACTGACACTATATATTATAAGAATATATCAGGATTTAAAACTGAAG
CCAAACTTGTTGGACTGGAACCAGTCAGCACCTACTCTATCCGTGTATCTGCGTTCACCAAAGTTGGA
AATGGCAATCAATTTAGTAATGTAGTAAAATTCACAACCCAAGAATCAGTTCCAGATGTCGTGCAGAA
TATGCAGTGCATGGCAACTAGCTGGCAGTCAGTTTTAGTGAAATGGGATCCACCCAAAAAGGCAAATG
GAATAATAACGCAGTATATGGTAACAGTTGAAAGGAATTCTACAAAAGTTTCTCCCCAAGATCACATG
TACACTTTCATAAAGCTTCTTGCCAATACCTCATATGTCTTTAAAGTAAGAGCTTCAACCTCAGCTGG
TGAAGGTGATGAAAGCACATGCCATGTCAGCACACTACCTGAAACAGTTCCCAGTGTTCCCACAAATA
TTGCTTTTTCTGATGTTCAGTCAACTAGTGCAACATTGACATGGATAAGACCTGACACTATCCTTGGC
TACTTTCAAAATTACAAAATTACCACTCAACTTCGTGCTCAAAAATGCAAAGAATGGGAATCCGAAGA
ATGTGTTGAATATCAAAAAATTCAATACCTCTATGAAGCTCACTTAACTGAAGAGACAGTATATGGAT
TAAAGAAATTTAGATGGTATAGATTCCAAGTGGCTGCCAGCACCAATGCTGGCTATGGCAATGCTTCA
AACTGGATTTCTACAAAAACTCTGCCTGGCCCTCCAGATGGTCCTCCTGAAAATGTTCATGTAGTAGC
AACATCACCTTTTAGCATCAGCATAAGCTGGAGTGAACCTGCTGTCATTACTGGACCAACATGTTATC
TGATTGATGTCAAATCGGTAGATAATGATGAATTTAATATATCCTTCATCAAGTCAAATGAAGAAAAT
AAAACCATAGAAATTAAAGATTTAGAAATATTCACAAGGTATTCTGTAGTGATCACTGCATTTACTGG
GAACATTAGTGCTGCATATGTAGAAGGGAAGTCAAGTGCTGAAATGATTGTTACTACTTTAGAATCAG
CCCCAAAGGACCCACCTAACAACATGACATTTCAGAAGATACCAGATGAAGTTACAAAATTTCAATTA
ACGTCCCTTCCTCCTTCTCAACCTAATGGAAATATCCAAGTATATCAAGCTCTGGTTTACCGAGAAGA
TGATCCTACTGCTGTCCAGATTCACAACCTCAGTATTATACAGAAAACCAACACATTCGTCATTGCAA
TGCTAGAAGGACTAAAAGGTGGACATACATACAATATCAGTGTTTACGCAGTCAATAGTGCTGGTGCA
GGTCCAAAGGTTCCGATGAGAATAACCATGGATATCAAAGCTCCAGCACGACCAAAAACCAAACCAAC
CCCTATTTATGATGCCACAGGAAAACTGCTTGTGACTTCAACAACAATTACAATCAGAATGCCAATAT
GTTACTACAGTGATGATCATGGACCAATAAAAAATGTACAAGTGCTTGTGACAGAAACAGGAGCTCAG
CATGATGGAAATGTAACAAAGTGGTATGATGCATATTTTAATAAAGCAAGGCCATATTTTACAAATGA
AGGCTTTCCTAACCCTCCATGTACAGAAGGAAAGACAAAGTTTAGTGGCAATGAAGAAATCTACATCA
TAGGTGCTGATAATGCATGCATGATTCCTGGCAATGAAGACAAAATTTGCAATGGACCACTGAAACCA
AAAAAGCAATACTTATTTAAATTTAGAGCTACAAATATTATGGGACAATTTACTGACTCTGATTATTC
TGACCCTGTTAAGACTTTAGGCGAAGGACTTTCAGAAAGAACCCTCGAG

NOV15f, CG50718-04

| | | | |
|---|---|---|---|
| Protein Sequence | SEQ ID NO: 176 | 851 aa | MW at 95001.6kD |

PEGFVGNLTYESISSTAINVSWVPPAQPNGLVFYYVSLILQQTPRHVRPPLVTYERSIYFDNLEKYTD
YILKITPSTEKGFSDTYTAQLYIKTEEDVPETSPIINTFKNLSSTSVLLSWDPPVKPNGAIISYDLTL
QGPNENYSFITSDNYIILEELSPFTLYSFFAAARTRKGLGPSSILFFYTDESVPLAPPQNLTLINCTS
DFVWLKWSPSPLPGGIVKVYSFKIHEHETDTIYYKNISGFKTEAKLVGLEPVSTYSIRVSAFTKVGNG
NQFSNVVKFTTQESVPDVVQNNQCMATSWQSVLVKWDPPKKANGIITQYMVTVERNSTKVSPQDHMYT
FIKLLANTSYVFKVRASTSAGEGDESTCHVSTLPETVPSVPTNIAFSDVQSTSATLTWIRPDTILGYF
QNYKITTQLRAQKCKEWESEECVEYQKIQYLYEAHLTEETVYGLKKFRWYRFQVAASTNAGYGNASNW
ISTKTLPGPPDGPPENVHVVATSPFSISISWSEPAVITGPTCYLIDVKSVDMDEFNISFIKSNEENKT
IEIKDLEIFTRYSVVITAFTGNISAAYVEGKSSAEMIVTTLESAPKDPPNNMTFQKIPDEVTKFQLTS
LPPSQPNGNIQVYQALVYREDDPTAVQIHNLSIIQKTNTFVIAMLEGLKGGHTYNISVYAVNSAGAGP
KVPMRITMDIKAPARPKTKPTPIYDATGKLLVTSTTITIRMPICYYSDDHGPIKNVQVLVTETGAQHD
GNVTKWYDAYFNKARPYFTNEGFPNPPCTEGKTKFSGNEEIYIIGADNACMIPGNEDKICNGPLKPKK
QYLFKFRATNIMGQFTDSDYSDPVKTLGEGLSERT

| | | |
|---|---|---|
| NOV15g, CG50718-05 | SEQ ID NO: 177 | 6903 bp |

| | | |
|---|---|---|
| DNA Sequence | ORF Start: ATG at 1 | ORF Stop: TAA at 6901 |

ATGGATTTTCTTATCATTTTTCTTTTACTTTTTATTGGGACTTCAGAGACACAGGTAGATGTTTCCAA
TGTCGTTCCTGGTACTAGGTACGATATAACCATCTCTTGAATTTCTACAACATACACCTCACCTGTTA
CTAGAATAGTGACAACAAATGTAACAGAACCAGGGCCTCCAGTCTTCCTAGCCGGGGAAAGAGTCGGA
TCTGCTGGGATTCTTCTGTCTTGGAATACACCACCTAATCCAAATGGAAGGATTATATCTTACATTGT
CAAATATAAGGAAGTTTGTCCGTGGATGCAAACAGTATATACACAAGTCAGATCAAAGCCAGACAGTC
TGGAAGTTCTTCTTACTAATCTTAATCCTGGAACAACATATGAAATTAAGGTAGCTGCTGAAAACAGT
GCTGGCATTGGAGTGTTTAGTGATCCATTTCTCTTCCAAACTGCAGAAAGTCCAGCTCCAGGAAAAGT
GGTGAATCTCACAGTTGAGGCCTACAACGCTTCAGCAGTTAAGCTGATTTGGTATTTACCTCGGCAAC
CAAATGGCAAAATTACCAGCTTCAAGATTAGTGTCAAGCATGCCAGAAGTGGGATAGTAGTGAAAGAT
GTCTCAATCAGAGTAGAGGACATTTTGACTGGGAAATTGCCAGAATGCAATGTAGAGAATAGTGAATC
TTTTTTATGGAGTACAGCCAGCCCTTCTCCAACCCTTGGTAGAGTTACACCTCCATCGCGTACCACAC
ATTCATCAAGCACGTTGACACAGAATGAGATCAGCTCTGTGTGGAAAGAGCCTATCAGTTTTGTAGTG
ACACACTTGAGACCTTATACAACATATCTTTTTGAAGTTTCAGCTGCTACAACTGAAGCAGGTTATAT
TGATAGTACGATTGTCAGAACACCAGAATCAGTGCCTGAAGGACCACCACAAAACTGCGTAACAGGCA
ACATCACAGGAAAGTCCTTTTCAATTTTATGGGACCCACCAACTATAGTAACAGGGAAATTTAGTTAT
AGAGTTGAATTATATGGACCATCAGGTCGCATTTTGGATAACAGCACAAAAGACCTCAAGTTTGCATT
CACTAACCTAACACCATTTACAATGTATGATGTCTATATTGCGGCTGAAACCAGTGCAGGGACTGGGC
CCAAGTCAAATATTTCAGTATTCACTCCACCAGATGTTCCAGGGGCAGTGTTTGATTTACAACTTGCA
GAGGTAGAATCCACGCAAGTAAGAATTACTTGGAAGAAACCACGACAACCAAATGGAATTATTAACCA
ATACCGAGTGAAAGTGCTAGTTCCAGAGACAGGAATAATTTTGGAAAATACTTTGCTCACTGGAAATA
ATGAGATAAATGACCCCATGGCTCCAGAAATTGTGAACATAGTAGAGCCAATGGTAGGATTATATGAG
GGTTCAGCAGAGATGTCGTCTGACCTTCACTCACTTGCTACATTTATATATAACAGCCATCCAGATAA
AAACTTTCCTGCAAGGAATAGAGCTGAAGACCAGACTTCACCAGTTGTAACTACAAGGAATCAGTATA
TTACTGACATTGCAGCTGAACAGCTGTCTTATGTTATCAGGAGACTTGTACCTTTCACTGAGCACATG
ATTAGTGTATCTGCTTTCACCATCATGGGAGAAGGACCACCAACAGTTCTCAGTGTTAGGACACGTCA
GCAAGTGCCAAGCTCCATTAAAATTATAAACTATAAAAATATTAGTTCTTCATCTATTTTGTTATATT
GGGATCCTCCAGAATATCCCAATGGAAAAATAACTCACTATACGATTTATGCAATGGAATTGGATACA
AACAGAGCATTCCAGATAACTACCATAGATAACAGCTTTCTCATAACAGGTATAGGGTTAAAGAAATA
CACAAAATACAAAATGAGAGTGGCAGCCTCAACCCACGTTGGAGAAAGTTCTTTGTCTGAAGAAAATG
ACATCTTTGTGAGAACTTCAGAAGATGAACCGGATCATCACCTCACCGATGTCGAAGTAATTGATGTT
ACCGCAGATGAAATAAGGTTGAAGTGGTCACCACCCGAAAAGCCCAATGGGATCATTATTGCTTATGA
AGTGCTATATAAAAATATAGATACTTTATATATGAAGAACACATCAACAACAGACATAATATTAAGGA
ACTTAAGACCTCACACCCTCTATAACATTTCTGTAAGGTCTTACACCAGATTTGGTCATGGCAATCAG
GTATCTTCTTTACTCTCTGTAAGGACTTCGGAGACTGTGCCTGATAGTGCACCAGAAAATATCACTTA
CAAAAATATTTCTTCTGGAGAGATTGAGCTATCATTCCTTCCCCCAAGTAGTCCCAATGGAATCATAC
AAAAATATACAATTTATCTCAAGAGAAGTAATGGAAATGAGGAAAGAACTATAAATACAACCTCTTTA
ACCCAAAACATTCTGAAGAAATATACCCAATATATCATTGAGGTGTCTGCTAGTACACTCAAAGGTGA
AGGAGTTCGGAGTGCTCCCATAAGTATACTGACGGAGGAAGATGCTCCTGATTCTCCCCCTCAAGACT
TCTCTGTAAAACAGTTGTCTGGTGTCACGGTGAAGTTGTCATGGCAACCACCCCTGGAGCCAAATGGA
ATTATCCTTTATTACACAGTTTATGTCTGGAGGAATAGATCATCATTAAAAACTATTAATGTCACTGA
AACATCATTGGAGTTATCAGATTTGGATTATAATGTTGAATACAGTGCTTATGTAACAGCTAGCACCA
GATTTGGTGATGGGAAAACAAGAAGCAATATCATTAGCTTTCAAACACCAGAGGGACCAAGCGATCCT
CCCAAAGATGTTTATTATGCAAACCTCAGTTCTTCATCAATAATTCTTTTCTGGACACCTCCTTCAAA
ACCTAATGGGATTATACAATATTACTCTGTTTATTACAGAATTACTTCAGGTACTTTTATGCAGAATT
TTACACTCCATGAAGTAACCAATGACTTTGACAATATGACTGTATCCACAATTATAGATAAACTGACA
ATATTCAGCTACTATACATTTTGGTTAACAGCAAGTACTTCAGTTGGAAATGGGAATAAAAGCAGTGA
CATCATTGAAGTATACACAGATCAAGACGTCCCTGAAGGGTTTGTTGGAAACCTGACTTACGAATCCA
TTTCGTCAACTGCAATAAATGTAAGCTGGGTCCCACCGGCTCAACCAAACGGTCTAGTCTTCTACTAT
GTTTCACTGATCTTACAGCAGACTCCTCGCCATGTGAGACCACCTCTTGTTACATATGAGAGAAGCAT
ATATTTTGATAATCTGGAAAAATACACTGATTATATATTAAAAATTACTCCATCAACAGAAAAGGGAT
TCTCTGATACCTATACTGCCCAGCTATACATCAAGACTGAAGAAGATGTCCCAGAAACTTCACCAATA
ATCAACACTTTTAAAAACCTTTCCTCTACCTCAGTTCTCTTATCATGGGATCCCCCAGTAAAGCCAAA
TGGTGCAATAATAAGTTATGATTTAACTTTACAAGGACCAAATGAAAATTATTCTTTCATTACTTCTG
ATAATTACATAATATTGGAAGAGCTTTCACCATTTACATTATATAGCTTTTTTGCTGCCGCAAGAACT
AGAAAAGGACTTGGTCCTTCCAGTATTCTTTTCTTTTACACAGATGAGTCAGTGCCGTTAGCACCTCC
ACAAAATTTGACTTTAATCAACTGTACTTCAGACTTTGTATGGCTGAAATGGAGCCCAAGTCCTCTTC
CAGGTGGTATTGTTAAAGTATATAGTTTTAAAATTCATGAACATGAAACTGACACTATATATTATAAG
AATATATCAGGATTTAAAACTGAAGCCAAACTTGTTGGACTGGAACCAGTCAGCACCTACTCTATCCG
TGTATCTGCGTTCACCAAAGTTGGAAATGGCAATCAATTTAGTAATGTAGTAAAATTCACAACCCAAG
AATCAGTTCCAGATGTCGTGCAGAATATGCAGTGCATGGCAACTAGCTGGCAGTCAGTTTTAGTGAAA
TGGGATCCACCCAAAAAGGCAAATGGAATAATAACGCAGTATATGGTAACAGTTGAAAGGAATTCTAC
AAAAGTTTCTCCCCAAGATCACATGTACACTTTCATAAAGCTTCTTGCCAATACCTCATATGTCTTTA
AAGTAAGAGCTTCAACCTCAGCTGGTGAAGGTGATGAAAGCACATGCCATGTCAGCACACTACCTGAA
ACAGTTCCCAGTGTTCCCACAAATATTGCTTTTTCTGATGTTCAGTCAACTAGTGCAACATTGACATG
GATAAGACCTGACACTATCCTTGGCTACTTTCAAAATTACAAAATTACCACTCAACTTCGTGCTCAAA
AATGCAAAGAATGGGAATCCGAAGAATGTGTTGAATATCAAAAAATTCAATACCTCTATGAAGCTCAC
TTAACTGAAGAGACAGTATATGGATTAAAGAAATTTAGATGGTATAGATTCCAAGTGGCTGCCAGCAC
CAATGCTGGCTATGGCAATGCTTCAAACTGGATTTCTACAAAAACTCTGCCTGGCCCTCCAGATGGTC
CTCCTGAAAATGTTCATGTAGTAGCAACATCACCTTTTAGCATCAGCATAAGCTGGAGTGAACCTGCT
GTCATTACTGGACCAACATGTTATCTGATTGATGTCAAATCGGTAGATAATGATGAATTTAATATATC
CTTCATCAAGTCAAATGAAGAAAATAAAACCATAGAAATTAAAGATTTAGAAATATTCACAAGGTATT
CTGTAGTGATCACTGCATTTACTGGGAACATTAGTGCTGCATATGTAGAAGGGAAGTCAAGTGCTGAA
ATGATTGTTACTACTTTAGAATCAGCCCCAAAGGACCCACCTAACAACATGACATTTCAGAAGATACC
AGATGAAGTTACAAAATTTCAATTAACGTCCCTTCCTCCTTCTCAACCTAATGGAAATATCCAAGTAT
ATCAAGCTCTGGTTTACCGAGAAGATGATCCTACTGCTGTCCAGATTCACAACCTCAGTATTATACAG
AAAACCAACACATTCGTCATTGCAATGCTAGAAGGACTAAAAGGTGGACATACATACAATATCAGTGT
TTACGCAGTCAATAGTGCTGGTGCAGGTCCAAAGGTTCCGATGAGAATAACCATGGATATCAAAGCTC
CAGCACGACCAAAAACCAAACCAACCCCTATTTATGATGCCACAGGAAAACTGCTTGTGACTTCAACA
ACAATTACAATCAGAATGCCAATATGTTACTACAGTGATGATCATGGACCAATAAAAAATGTACAAGT
GCTTGTGACAGAAACAGGAGCTCAGCATGATGGAAATGTAACAAAGTGGTATGATGCATATTTTAATA
AAGCAAGGCCATATTTTACAAATGAAGGCTTTCCTAACCCTCCATGTACAGAAGGAAAGACAAAGTTT
AGTGGCAATGAAGAAATCTACATCATAGGTGCTGATAATGCATGCATGATTCCTGGCAATGAAGACAA
AATTTGCAATGGACCACTGAAACCAAAAAAGCAATACTTATTTAAATTTAGAGCTACAAATATTATGG
GACAATTTACTGACTCTGATTATTCTGACCCTGTTAAGACTTTAGGCGAAGGACTTTCAGAAAGAACC
CTAGAGATCATTCTTTCCGTCACTTTGTGTATCCTTTCAATAATTCTCCTTGGAACAGCTATTTTTGC
ATTTGCAAGAATTCGACAGAAGCAGAAAGAAGGTGGCACATACTCTCCTCAGGATGCAGAAATTATTG
ACACTAAATTGAAGCTGGATCAGCTCATCACAGTGGCAGACCTGGAACTGAAGGACGAGAGATTAACG
CGGTTACTTAGTTATAGAAAATCCATCAAGCCAATAAGCAAGAAATCCTTCCTGCAACATGTTGAAGA
GCTTTGCACAAACAACAACCTAAAGTTTCAAGAAGAATTTTCGGAATTACCAAAATTTCTTCAGGATC
TTTCTTCAACTGATGCTGATCTGCCTTGGAATAGAGCAAAAAACCGCTTCCCAAACATAAAACCATAT
AATAATAACAGAGTAAAGCTGATAGCTGACGCTAGTGTTCCAGGTTCGGATTATATTAATGCCAGCTA
TATTTCTGGTTATTTATGTCCAAATGAATTTATTGCTACTCAAGGTCCACTACCAGGAACAGTTGGAG
ATTTTTGGAGAATGGTGTGGGAAACCAGAGCAAAAACATTAGTAATGCTAACACAGTGTTTTGAAAAA
GGACGGATCAGATGCCATCAGTATTGGCCAGAGGACAACAAGCCAGTTACTGTCTTTGGAGATATAGT
GATTACAAAGCTAATGGAGGATGTTCAAATAGATTGGACTATCAGGGATCTGAAAATTGAAAGGCATG
GGGATTGCATGACTGTTCGACAGTGTAACTTTACTGCCTGGCCAGAGCATGGGGTTCCTGAGAACAGC
GCCCCTCTAATTCACTTTGTGAAGTTGGTTCGAGCAAGCAGGGCACATGACACCACACCTATGATTGT
TCACTGTAGTGCTGGAGTTGGAAGAACTGGAGTTTTTATTGCTCTGGACCATTTAACACAACATATAA
ATGACCATGATTTTGTGGATATATATGGACTAGTAGCTGAACTGAGAAGTGAAAGAATGTGCATGGTG
CAGAATCTGGCACAGTATATCTTTTTACACCAGTGCATTCTGGATCTCTTATCAAATAAGGGAAGTAA
TCAGCCCATCTGTTTTGTTAACTATTCAGCACTTCAGAAGATGGACTCTTTGGACGCCATGGAAGGTG
ATGTTGAGCTTGAATGGGAAGAAACCACTATGTAA

NOV15g, CG50718-05

| | | | |
|---|---|---|---|
| Protein Sequence | SEQ ID NO: 178 | 2300 aa | MW at 257261.9kD |

MDFLIIFLLLFIGTSETQVDVSNVVPGTRYDITISSISTTYTSPVTRIVTTNVTEPGPPVFLAGERVG
SAGILLSWNTPPNPNGRIISYIVKYKEVCPWMQTVYTQVRSKPDSLEVLLTNLNPGTTYEIKVAAENS
AGIGVFSDPFLFQTAESPAPGKVVNLTVEAYNASAVKLIWYLPRQPNGKITSFKISVKHARSGIVVKD
VSIRVEDILTGKLPECNVENSESFLWSTASPSPTLGRVTPPSRTTHSSSTLTQNEISSVWKEPISFVV
THLRPYTTYLFEVSAATTEAGYIDSTIVRTPESVPEGPPQNCVTGNITGKSFSILWDPPTIVTGKFSY
RVELYGPSGRILDNSTKDLKFAFTNLTPFTMYDVYIAAETSAGTGPKSNISVFTPPDVPGAVFDLQLA
EVESTQVRITWKKPRQPNGIINQYRVKVLVPETGIILENTLLTGNNEINDPMAPEIVNIVEPMVGLYE
GSAEMSSDLHSLATFIYNSHPDKNFPARNRAEDQTSPVVTTRNQYITDIAAEQLSYVIRRIVPFTEHM
ISVSAFTIMGEGPPTVLSVRTRQQVPSSIKIINYKNISSSSILLYWDPPEYPNGKITHYTIYAMELDT
NRAFQITTIDNSFLITGIGLKKYTKYKMRVAASTHVGESSLSEENDIFVRTSEDEPESSPQDVEVIDV
TADEIRLKWSPPEKPNGIIIAYEVLYKNIDTLYMKNTSTTDIILRNLRPHTLYNISVRSYTRFGHGNQ
VSSLLSVRTSETVPDSAPENITYKNISSGEIELSFLPPSSPNGIIQKYTIYLKRSNGNEERTINTTSL
TQNILKKYTQYIIEVSASTLKGEGVRSAPISILTEEDAPDSPPQDFSVKQLSGVTVKLSWQPPLEPNG
IILYYTVYVWRNRSSLKTINVTETSLELSDLDYNVEYSAYVTASTRFGDGKTRSNIISFQTPEGPSDP
PKDVYYANLSSSSIILFWTPPSKPNGIIQYYSVYYRNTSGTFMQNFTLHEVTNDFDNMTVSTIIDKLT
IFSYYTFWLTASTSVGNGNKSSDIIEVYTDQDVPEGFVGNLTYESISSTAINVSWVPPAQPNGLVFYY
VSLILQQTPRHVRPPLVTYERSIYFDNLEKYTDYILKITPSTEKGFSDTYTAQLYIKTEEDVPETSPI
INTFKNLSSTSVLLSWDPPVKPNGAIISYDLTLQGPNENYSFITSDNYIILEELSPFTLYSFFAAART
RKGLGPSSILFFYTDESVPLAPPQNLTLINCTSDFVWLKWSPSPLPGGIVKVYSFKIHEHETDTIYYK
NISGFKTEAKLVGLEPVSTYSIRVSAFTKVGNGNQFSNVVKFTTQESVPDVVQNNQCMATSWQSVLVK
WDPPKKANGIITQYMVTVERNSTKVSPQDHMYTFIKLLANTSYVFKVRASTSAGEGDESTCHVSTLPE
TVPSVPTNIAFSDVQSTSATLTWIRPDTILGYFQNYKITTQLRAQKCKEWESEECVEYQKIQYLYEAH
LTEETVYGLKKFRWYRFQVAASTNAGYGNASNWISTKTLPGPPDGPPENVHVVATSPFSISISWSEPA
VITGPTCYLIDVKSVDNDEFNISFIKSNEENKTIEIKDLEIFTRYSVVITAFTGNISAAYVEGKSSAE
MIVTTLESAPKDPPNNMTFQKIPDEVTKFQLTSLPPSQPNGNIQVYQALVYREDDPTAVQIHNLSIIQ
KThTFVIAMLEGLKGGHTYNISVYAVNSAGAGPKVPMRITMDIKAPARPKTKPTPIYDATGKLLVTST
TITIRMPICYYSDDHGPIKNVQVLVTETGAQHDGNVTKWYDAYFNKARPYFTNEGFPNPPCTEGKTKF
SGNEEIYIIGADNACMIPGNEDKICNGPLKPKKQYLFKFRATNIMGQFTDSDYSDPVKTLGEGLSERT
LEIILSVTLCILSIILLGTAIFAFARIRQKQKEGGTYSPQDAEIIDTKLKLDQLITVADLELKDERLT
RLLSYRKSIKPISKKSFLQHVEELCTNNNLKFQEEFSELPKFLQDLSSTDADLPWNRAKNRFPNIKPY
NNNRVKLIADASVPGSDYINASYISGYLCPNEFIATQGPLPGTVGDFWRMVWETRAKTLVMLTQCFEK
GRIRCHQYWPEDNKPVTVFGDIVITKLMEDVQIDWTIRDLKIERHGDCMTVRQCNFTAWPEHGVPENS
APLIHFVKLVRASRAHDTTPMIVHCSAGVGRTGVFIALDHLTQHINDHDFVDIYGLVAELRSERMCMV
QNLAQYIFLHQCILDLLSNKGSNQPICFVNYSALQKMDSLDAMEGDVELEWEETTM

A ClustalW comparison of the above protein sequences yields the following sequence alignment shown in Table 15B.

TABLE 15B
Comparison of the NOV15 protein sequences.

| | |
|---|---|
| NOV15a | MDFLIIFLLLFIGTSETQVDVSNVVPGTRYDITISSISTTYTSPVTRIGASNEPGPPVFL |
| NOV15b | ------------------------------------------------------------ |
| NOV15c | ------------------------------------------------------------ |
| NOV15d | MDFLIIFLLLFIGTSETQVDVSNVVPGTRYDITISSISTTYTSPVTRIGASNEPGPPVFL |
| NOV15e | ------------------------------------------------------------ |
| NOV15f | ------------------------------------------------------------ |
| NOV15g | ------------------------------------------------------------ |
| NOV15a | AGERVGSAGILLSWNTPPNPNGRIISYIVKYKEVCPWMQTVYTQVRSKPDSLEVLLTNLN |
| NOV15b | ------------------------------------------------------------ |
| NOV15c | ------------------------------------------------------------ |
| NOV15d | AGERVGSAGILLSWNTPPNPNGRIISYIVKYKEVCPWMQTVYTQVRSKPDSLEVLLTNLN |
| NOV15e | ------------------------------------------------------------ |
| NOV15f | ------------------------------------------------------------ |
| NOV15g | ------------------------------------------------------------ |
| NOV15a | PGTTYEIKVAAENSAGIGVFSDPFLFQTAESAPGKVVDFTGEAVPFSSKLMWYTSATKKK |
| NOV15b | ------------------------------------------------------------ |
| NOV15c | ------------------------------------------------------------ |
| NOV15d | PGTTYEIKVAAENSAGIGVFSDPFLFQTAESAPGKVVDFTGEAVPFSSKLMWYTSATKKK |
| NOV15e | ------------------------------------------------------------ |
| NOV15f | ------------------------------------------------------------ |
| NOV15g | ------------------------------------------------------------ |
| NOV15a | ITSFKISVKHNRSGIVVKEVSIRVECILSASLPLHCNENSESFLWSTASPSPTLGRVTPP |
| NOV15b | ------------------------------------------------------------ |
| NOV15c | ------------------------------------------------------------ |
| NOV15d | ITSFKISVKHNRSGIVVKEVSIRVECILSASLPLHCNENSESFLWSTASPSPTLGRVTPP |
| NOV15e | ------------------------------------------------------------ |
| NOV15f | ------------------------------------------------------------ |
| NOV15g | ------------------------------------------------------------ |
| NOV15a | SRTTHSSSTLTQNEISSVKEPISFVVTHLRPYTTYLFEVSAATTEAGYIDSTIVRTPESV |
| NOV15b | ------------------------------------------------------------ |
| NOV15c | ------------------------------------------------------------ |
| NOV15d | SRTTHSSSTLTQNEISSVKEPISFVVTHLRPYTTYLFEVSAATTEAGYIDSTIVRTPESV |
| NOV15e | ------------------------------------------------------------ |
| NOV15f | ------------------------------------------------------------ |
| NOV15g | ------------------------------------------------------------ |
| NOV15a | PEGPPQNCVTGNITGKSFSILWDPPTIVTGKFSYRVELYGPSAGRILDNSTKDLKFAFTN |
| NOV15b | ------------------------------------------------------------ |
| NOV15c | ------------------------------------------------------------ |
| NOV15d | PEGPPQNCVTGNITGKSFSILWDPPTIVTGKFSYRVELYGPSAGRILDNSTKDLKFAFTN |
| NOV15e | ------------------------------------------------------------ |
| NOV15f | ------------------------------------------------------------ |
| NOV15g | ------------------------------------------------------------ |
| NOV15a | LTPFTMYDVYIAAETSAGTGPKSNISVFTPPDVPGAVFDLQLAEVESTQVRITWKKPRQP |
| NOV15b | ------------------------------------------------------------ |
| NOV15c | ------------------------------------------------------------ |
| NOV15d | LTPFTMYDVYIAAETSAGTGPKSNISVFTPPDVPGAVFDLQLAEVESTQVRITWKKPRQP |
| NOV15e | ------------------------------------------------------------ |
| NOV15f | ------------------------------------------------------------ |
| NOV15g | ------------------------------------------------------------ |
| NOV15a | NGIINQYRVKVLVPETGIILENTLLTGNNEINDPMAPEIVNIVQPMVGLYEGSAEMSSDL |
| NOV15b | ------------------------------------------------------------ |
| NOV15c | ------------------------------------------------------------ |
| NOV15d | NGIINQYRVKVLVPETGIILENTLLTGNNEINDPMAPEIVNIVQPMVGLYEGSAEMSSDL |
| NOV15e | ------------------------------------------------------------ |
| NOV15f | ------------------------------------------------------------ |
| NOV15g | ------------------------------------------------------------ |
| NOV15a | HSLATFIYNSHPDKNFPARNRAEDQTSPVVTTRNQYITDIAAEQLTYVLIRLRRFWAETM |
| NOV15b | ------------------------------------------------------------ |
| NOV15c | ------------------------------------------------------------ |
| NOV15d | HSLATFIYNSHPDKNFPARNRAEDQTSPVVTTRNQYITDIAAEQLTYVLIRLRRFWAETM |
| NOV15e | ------------------------------------------------------------ |
| NOV15f | ------------------------------------------------------------ |
| NOV15g | ------------------------------------------------------------ |
| NOV15a | GFSRYTIMSSASRDNLTSPGPLSAQNFRVTHVTITEVFLHWDPPDPVFFHHYLITILDVE |
| NOV15b | ------------------------------------------------------------ |
| NOV15c | ------------------------------------------------------------ |
| NOV15d | GFSRYTIMSSASRDNLTSPGPLSAQNFRVTHVTITEVFLHWDPPDPVFFHHYLITILDVE |
| NOV15e | ------------------------------------------------------------ |
| NOV15f | ------------------------------------------------------------ |
| NOV15g | ------------------------------------------------------------ |
| NOV15a | NQSKSIILRTLNSLSLVLIGLKKYTKYKMRVAASTHVGESSLSEENDIFVRTSEDEPESS |
| NOV15b | ------------------------------------------------------------ |
| NOV15c | ------------------------------------------------------------ |
| NOV15d | NQSKSIILRTLNSLSLVLIGLKKYTKYKMRVAASTHVGESSLSEENDIFVRTSEDEPESS |
| NOV15e | ------------------------------------------------------------ |
| NOV15f | ------------------------------------------------------------ |
| NOV15g | ------------------------------------------------------------ |
| NOV15a | PQDVEVIDVTADEIRLKWSPPEKPNGIIIAYEVLYKNIDTLYNKNTSTTDIILRNLRPHT |
| NOV15b | ------------------------------------------------------------ |
| NOV15c | ------------------------------------------------------------ |
| NOV15d | PQDVEVIDVTADEIRLKWSPPEKPNGIIIAYEVLYKNIDTLYNKNTSTTDIILRNLRPHT |
| NOV15e | ------------------------------------------------------------ |
| NOV15f | ------------------------------------------------------------ |
| NOV15g | ------------------------------------------------------------ |
| NOV15a | LYNISVRSYTRFGHGNQVSSLLSVRTSESVPDSAPENITYKNISSGEIELSFLPPSSPNG |
| NOV15b | ------------------------------------------------------------ |
| NOV15c | ------------------------------------------------------------ |
| NOV15d | LYNISVRSYTRFGHGNQVSSLLSVRTSESVPDSAPENITYKNISSGEIELSFLPPSSPNG |
| NOV15e | ------------------------------------------------------------ |
| NOV15f | ------------------------------------------------------------ |
| NOV15g | ------------------------------------------------------------ |
| NOV15a | IIQKYTIYLKRSNGNEERTINTTSLTQNIKGLKKYTQYIIEVSASTLKGEGVRSAPISIL |
| NOV15b | ------------------------------------------------------------ |
| NOV15c | ------------------------------------------------------------ |
| NOV15d | IIQKYTIYLKRSNGNEERTINTTSLTQNIKGLKKYTQYIIEVSASTLKGEGVRSAPISIL |
| NOV15e | ------------------------------------------------------------ |
| NOV15f | ------------------------------------------------------------ |
| NOV15g | ------------------------------------------------------------ |
| NOV15a | TEEDAPDSPPQDFSVKQLSGVTVKLSWQPPLEPNGIILYYTVYVWRSSLKTINVTETSLE |
| NOV15b | ------------------------------------------------MDFLIIFLLLFI |
| NOV15c | ------------------------------------------------------------ |
| NOV15d | TEEDAPDSPPQDFSVKQLSGVTVKLSWQPPLEPNGIILYYTVYVWRSSLKTINVTETSLE |
| NOV15e | ------------------------------------------------------------ |
| NOV15f | ------------------------------------------------------------ |
| NOV15g | ------------------------------------------------MDFLIIFLLLFI |
| NOV15a | LSDLDYNVEYSAYVTASTRFGDGKTRSNIISFQTPEGPSDPPKDVYYANLSSSSIILFWT |
| NOV15b | GTSETQVDVSNVVPGTRYDITISSISTTYTSPVTRIVTTNVTKPGPPVFLAGERVGSAGI |
| NOV15c | ------------------------------------------------------------ |
| NOV15d | LSDLDYNVEYSAYVTASTRFGDGKTRSNIISFQTPEGPSDPPKDVYYANLSSSSIILFWT |
| NOV15e | -----QVDVSNVVPGTRYDITISSISTTYTSPVTRIVTTNVTKPGPPVFLAGERVGSAGI |
| NOV15f | ------------------------------------------------------------ |
| NOV15g | GTSETQVDVSNVVPGTRYDITISSISTTYTSPVTRIVTTNVTEPGPPVFLAGERVGSAGI |
| NOV15a | PPSKPNGIIQYYSVYYRNTSGTFMQNFTLHEVTNDFDNMTVSTIIDKLTIFSYYTFWLTA |
| NOV15b | LLSWNTPPNPMGRIISYIVKYKEVCPWMQTVYTQVRSKPDSLEVLLTNLNPGTTYEIKVA |
| NOV15c | ------------------------------------------------------------ |
| NOV15d | PPSKPNGIIQYYSVYYRNTSGTFMQNFTLHEVTNDFDNMTVSTIIDKLTIFSYYTFWLTA |
| NOV15e | LLSWNTPPNPMGRIISYIVKYKEVCPWMQTVYTQVRSKPDSLEVLLTNLNPGTTYEIKVA |
| NOV15f | ------------------------------------------------------------ |
| NOV15g | LLSWNTPPNPMGRIISYIVKYKEVCPWMQTVYTQVRSKPDSLEVLLTNLNPGTTYEIKVA |
| NOV15a | STSVGNGNKSSDIIEVYTDQDVPEGFVGNLTYESISSTAINVSWVPPAQPNG-------- |
| NOV15b | AENSAGIGVFSDPFLFQTAES-APGKVVNLTVEAYNASAVKLIWYLPRQPNGKITSFKIS |
| NOV15c | ------------------------------------------------------------ |
| NOV15d | STSVGNGNKSSDIIEVYTDQDVPEGFVGNLTYESISSTAINVSWVPPAQPNG-------- |
| NOV15e | AENSAGIGVFSDPFLFQTAES-APGKVVNLTVEAYNASAVKLIWYLPRQPNGKITSFKIS |
| NOV15f | ----------------------PEGFVGNLTYESISSTAINVSWVPPAQPNG-------- |
| NOV15g | AENSAGIGVFSDPFLFQTAESPAPGKVVNLTVEAYNASAVKLIWYLPRQPNGKITSFKIS |
| NOV15a | -------------------------LVFYY--------VSLILQQTPRHVRPPLVTYERS |
| NOV15b | VKHARSGIVVKDVSIRVEDILTGKLPECNE-NSESFLWSTASPSPTLGRVTPPSRTTHSS |
| NOV15c | ------------------------------------------------------------ |
| NOV15d | -------------------------LVFYY--------VSLILQQTPRHVRPPLVTYERS |
| NOV15e | VKHARSGIVVKDVSIRVEDILTGKLPECNE-NSESFLWSTASPSPTLGRVTPPSRTTHSS |
| NOV15f | -------------------------LVFYY--------VSLILQQTPRHVRPPLVTYERS |
| NOV15g | VKHARSGIVVKDVSIRVEDILTGKLPECNVENSESFLWSTASPSPTLGRVTPPSRTTHSS |
| NOV15a | IYFDN--------------LEKYTDYILKITPSTEKGFSDTYTAQLYIKTEEDIPETSPI |
| NOV15b | STLTQNEISSVWKEPISFVVTHLRPYTTYLFEVSAVTTEAGYIDSTIVRTPESVPEGPPQ |
| NOV15c | ----------------------------------------------------RSPEGPPQ |
| NOV15d | IYFDN--------------LEKYTDYILKITPSTEKGFSDTYTAQLYIKTEEDIPETSPI |
| NOV15e | STLTQNEISSVWKEPISFVVTHLRPYTTYLFEVSAVTTEAGYIDSTIVRTPESVPEGPPQ |
| NOV15f | IYFDN--------------LEKYTDYILKITPSTEKGFSDTYTAQLYIKTEEDVPETSPI |
| NOV15g | STLTQNEISSVWKEPISFVVTHLRPYTTYLFEVSAATTEAGYIDSTIVRTPESVPEGPPQ |
| NOV15a | INTFKNLSSTSVLLSWDPPVKPNGAIISYDLTLQGPN-ENYSFITSDNYIILEELSPFTL |
| NOV15b | NCVTGNITGKSFSILWDPPTIVT-GKFSYRVELYGPSGRILDNSTKDLKFAFTNLTPFTM |
| NOV15c | NCVTGNITGKSFSILWDPPTIVT-GKFSYRVELYGPSGRILDNSTKDLKFAFTNLTPFTM |
| NOV15d | INTFKNLSSTSVLLSWDPPVKPNGAIISYDLTLQGPN-ENYSFITSDNYIILEELSPFTL |
| NOV15e | NCVTGNITGKSFSILWDPPTIVT-GKFSYRVELYGPSGRILDNSTKDLKFAFTNLTPFTM |
| NOV15f | INTFKNLSSTSVLLSWDPPVKPNGAIISYDLTLQGPN-ENYSFITSDNYIILEELSPFTL |
| NOV15g | NCVTGMITGKSFSILWDPPTIVT-GKFSYRVELYGPSGRILDNSTKDLKFAFThLTPFTM |
| NOV15a | YSFFAAARTRKGLGPSSILFFYTDESVPLAPPQNLTLINCTSDFVWLKWSPSPLPGGIVK |
| NOV15b | YDVYIAAETSAGTGPKSNISVFTPPDVPGA-VFDLQLAEVESTQVRITWKKPRQPNGIIN |
| NOV15c | YDVYIAAETSAGTGPKSNISVFTPPDVPGA-VFDLQLAEVESTQVRITWKKPRQPNGIIN |
| NOV15d | YSFFAAARTRKGLGPSSILFFYTDESVPLAPPQNLTLINCTSDFVWLKWSPSPLPGGIVK |
| NOV15e | YDVYIAAETSAGTGPKSNISVFTPPDVPGA-VFDLQLAEVESTQVRITWKKPRQPNGIIN |
| NOV15f | YSFFAAARTRKGLGPSSILFFYTDESVPLAPPQNLTLINCTSDFVWLKWSPSPLPGGIVK |
| NOV15g | YDVYIAAETSAGTGPKSNISVFTPPDVPGA-VFDLQLAEVESTQVRITWKKPRQPNGIIN |
| NOV15a | VYSFKIHEHETDTIYYKNISGFKTEAKLVGLEPVSTYSIRVSAFTKVGNGNQFSNVVKFT |
| NOV15b | QYRVKVLVPETGIILENTLLTGNNEYINDPMAPEIVNIVEPMVGLYEGSAEMSSDLHSLA |
| NOV15c | QYRVKVLVPETGIILENTLLTGNNEYINDPMAPEIVNIVEPMVGLYEGSAEMSSDLHSLA |
| NOV15d | VYSFKIHEHETDTIYYKNISGFKTEAKLVGLEPVSTYSIRVSAFTKVGNGNQFSNVVKFT |
| NOV15e | QYRVKVLVPETGIILENTLLTGNNEYINDPMAPEIVNIVEPMVGLYEGSAEMSSDLHSLA |
| NOV15f | VYSFKIHEHETDTIYYKNISGFKTEAKLVGLEPVSTYSIRVSAFTKVGNGNQFSNVVKFT |
| NOV15g | QYRVKVLVPETGIILENTLLTGNN-EINDPMAPEIVNIVEPMVGLYEGSAEMSSDLHSLA |
| NOV15a | TQESVPDVVQNNQCMATSWQSVLVKWDPPKKANGIITQYMVTVERNSTKVSPQDHMYTFI |
| NOV15b | TFIYNSHPDK--------------NFPARNRAEDQTSPVVTTRNQYITDIAAEQLSYVIR |
| NOV15c | TFIYNSHPDK--------------NFPARNRAEDQTSPVVTTRNQYITDIAAEQLSYVIR |
| NOV15d | TQESVPDVVQNNQCMATSWQSVLVKWDPPKKANGIITQYMVTVERNSTKVSPQDHMYTFI |
| NOV15e | TFIYNSHPDK--------------NFPARNRAEDQTSPVVTTRNQYITDIAAEQLSYVIR |
| NOV15f | TQESVPDVVQNMQCMATSWQSVLVKWDPPKKANGIITQYMVTVERNSTKVSPQDHMYTFI |
| NOV15g | TFIYNSHPDK--------------NFPARNRAEDQTSPVVTTRNQYITDIAAEQLSYVIR |
| NOV15a | KLLANTSYVFKVRASTSAGEGDESTCHVSTLPETVPSVPTNIAFSDVQSTSATLTWIRPD |
| NOV15b | RLVPFTEHMISVSAFTIMGEG-PPTVLSVRTRQQVPSSIKIINYKNISSSSILLYWDPPE |
| NOV15c | RLVPFTEHMISVSAFTIMGEG-PPTVLSVRTRQQVPSSIKIINYKNISSSSILLYWDPPE |
| NOV15d | KLLANTSYVFKVRASTSAGEGDESTCHVSTLPETVPSVPTNIAFSDVQSTSATLTWIRPD |
| NOV15e | RLVPFTEHMISVSAFTIMGEG-PPTVLSVRTRQQVPSSIKIINYKNISSSSILLYWDPPE |
| NOV15f | KLLANTSYVFKVRASTSAGEGDESTCHVSTLPETVPSVPTNIAFSDVQSTSATLTWIRPD |
| NOV15g | RLVPFTEHMISVSAFTIMGEG-PPTVLSVRTRQQVPSSIKIINYKNISSSSILLYWDPPE |
| NOV15a | TILGYFQNYKITTQLRAQKCKEWESEECVEYQKIQYLYEAHLTEETVYGLKKFRWYRFQV |
| NOV15b | YPNGKITHYTIY------------AMELDTNRAFQITTIDNSFLIT--GLKKYTKYKMRV |
| NOV15c | YPNGKITHYTIY------------AMELDTNRAFQITTIDNSFLIT--GLKKYTKYKMRV |
| NOV15d | TILGYFQNYKITTQLRAQKCKEWESEECVEYQKIQYLYEAHLTEETVYGLKKFRWYRFQV |
| NOV15e | YPNGKITHYTIY------------AMELDTNRAFQITTIDNSFLIT--GLKKYTKYKMRV |
| NOV15f | TILGYFQNYKITTQLRAQKCKEWESEECVEYQKIQYLYEAHLTEETVYGLKKFRWYRFQV |
| NOV15g | YPNGKITHYTIY------------AMELDTNRAFQITTIDNSFLITGIGLKKYTKYKMRV |
| NOV15a | AASTNAGYGNAS--NWISTKTLPGPPDGPPENVHVVATSPFSISISWSEPAVITGPTCYL |
| NOV15b | AASTHDGESSLSEENDIFVRTSEDEPESSPQDVEVIDVTADEIRLKWSPPEKPNG----I |
| NOV15c | AASTHDGESSLSEENDIFVRTSEDEPESSPQDVEVIDVTADEIRLKWSPPEKPNG----I |
| NOV15d | AASTNAGYGNAS--NWISTKTLPGPPDGPPENVHVVATSPFSISISWSEPAVITGPTCYL |
| NOV15e | AASTHDGESSLSEENDIFVRTSEDEPESSPQDVEVIDVTADEIRLKWSPPEKPNG----I |
| NOV15f | AASTNAGYGNAS--NWISTKTLPGPPDGPPENVHVVATSPFSISISWSEPAVITGPTCYL |
| NOV15g | AASTHVGESSLSEENDIFVRTSEDEPESSPQDVEVIDVTADEIRLKWSPPEKPNG----I |
| NOV15a | IDVKSVDNDEFNISFIKSNEENKTIEIKDLEIFTRYSVVITAFTGNISAAYVEGKSSAEM |
| NOV15b | IIAYEVLYKNIDTLYNKSNTSTTDIILRNLRPHTLYNISVRSYTRFGHGNQVS----SLL |
| NOV15c | IIAYEVLYKNIDTLYMK-NTSTTDIILRNLRPHTLYNISVRSYTRFGHGNQVS----SLL |
| NOV15d | IDVKSVDNDEFNISFIKSNEENKTIEIKDLEIFTRYSVVITAFTGNISAAYVEGKSSAEM |
| NOV15e | IIAYEVLYKNIDTLYMK-NTSTTDIILRNLRPHTLYNISVRSYTRFGHGNQVS----SLL |
| NOV15f | IDVKSVDNDEFNISFIKSNEENKTIEIKDLEIFTRYSVVITAFTGNISAAYVEGKSSAEM |
| NOV15g | IIAYEVLYKNIDTLYMK-NTSTTDIILRNLRPHTLYNISVRSYTRFGHGNQVS----SLL |
| NOV15a | IVTTLESAPKDPPNNMTFQKIPDEVTKFQLTFLPPSQPNGNIQVYQALVYREDDPTAVQI |
| NOV15b | SVRTSETVPDSAPENITYKNIS--SGEIELSFLPPSSPNGIIKKYTIYLKRSNGNEERTI |
| NOV15c | SVRTSETVPDSAPENITYKNIS--SGEIELSFLPPSSPNGIIKKYTIYLKRSNGNEERTI |
| NOV15d | IVTTLESAPKDPPNNMTFQKIPDEVTKFQLTFLPPSQPNGNIQVYQALVYREDDPTAVQI |
| NOV15e | SVRTSETVPDSAPENITYKNIS--SGEIELSFLPPSSPNGIIQKYTIYLKRSNGNEERTI |
| NOV15f | IVTTLESAPKDPPNNNTFQKIPDEVTKFQLTSLPPSQPNGNIQVYQALVYREDDPTAVQI |
| NOV15g | SVRTSETVPDSAPENITYKNIS--SGEIELSFLPPSSPNGIIQKYTIYLKRSNGNEERTI |
| NOV15a | HNLSIIQKTNTFVIAMLEGLKGGHTYNISVYAVNSAG-AGPKVPMRITMDIKAP-ARPKT |
| NOV15b | NTTSLTQNIK--------VLKKYTQYIIEVSASTLKGEGVRSAPISILTEEDAPDSPPQD |
| NOV15c | NTTSLTQNIK--------VLKKYTQYIIEVSASTLKGEGVRSAPISILTEEDAPDSPPQD |
| NOV15d | HNLSIIQKTNTFVIAMLEGLKGGHTYNISVYAVNSAG-AGPKVPMRITMDIKAP-ARPKT |
| NOV15e | NTTSLTQNIK--------GLKKYTQYIIEVSASTLKGEGVRSAPISILTEEDAPDSPPQD |
| NOV15f | HNLSIIQKTNTFVIAMLEGLKGGHTYNISVYAVNSAG-AGPKVPMRITMDIKAP-ARPKT |
| NOV15g | NTTSLTQNI----------LKKYTQYIIEVSASTLKGEGVRSAPISILTEEDAPDSPPQD |
| NOV15a | KPTPIYDATGKLLVTSTTITIRMPICYYSDDHGP--IKNVQVLVTETGAQH-DGNVTKWY |
| NOV15b | FSVKQLSGVTVKLSWQPPLEPNGIILYYTVYVWN-RSSLKTINVTETSLELSDLDYNVEY |
| NOV15c | FSVKQLSGVTVKLSWQPPLEPNGIILYYTVYVWN-RSSLKTINVTETSLELSDLDYNVEY |
| NOV15d | KPTPIYDATGKLLVTSTTITIRMPICYYSDDHGP--IKNVQVLVTETGAQH-DGNVTKWY |
| NOV15e | FSVKQLSGVTVKLSWQPPLEPNGIILYYTVYVWN-RSSLKTINVTETSLELSDLDYNVEY |
| NOV15f | KPTPIYDATGKLLVTSTTITIRMPICYYSDDHGP--IKNVQVLVTETGAQH-DGNVTKWY |
| NOV15g | FSVKQLSGVTVKLSWQPPLEPNGIILYYTVYVWRNRSSLKTINVTETSLELSDLDYNVEY |
| NOV15a | DAYFNKARPYFTNEGFPNPP--CT-EGKTKFSGNEEIYIIGADNACMIPGNEDKICNGPL |
| NOV15b | SAYVTASTRFGDGKTRSNIISFQTPEGAPSDPPKDVYYANLSSSSIILFWTPPSKPNGII |
| NOV15c | SAYVTASTRFGDGKTRSNIISFQTPEGAPSDPPKDVYYANLSSSSIILFWTPPSKPNGII |
| NOV15d | DAYFNKARPYFTNEGFPNPP--CT-EGKTKFSGNEEIYIIGADNACMIPGNEDKICNGPL |
| NOV15e | SAYVTAS---------------------------------------------------- |
| NOV15f | DAYFNKARPYFTNEGFPNPP--CT-EGKTKFSGNEEIYIIGADNACMIPGNEDKICNGPL |
| NOV15g | SAYVTASTRFGDGKTRSNIISFQTPEGP-SDPPKDVYYANLSSSSIILFWTPPSKPNGII |
| NOV15a | KPKKQYLFKFRATNIMGQFTDSDYSDPVKTLGEGLSERTVEIILSVTLCILSIILLGTAI |
| NOV15b | QYYSVYYRNTSGTFNQNFTLHEVTNDFDNNTVSTIIDKLTIFSYYTFWLTASTSVGNGNK |
| NOV15c | QYYSVYYRNTSGTFMQNFTLHEVTNDFDNNTVSTIIDKLTIFSYYTFWLTASTSVGNGNK |
| NOV15d | KPKKQYLFKFRATNIMGQFTDSDYSDPVKTLGEGLSERTVEIILSVTLCILSIILLGTAI |
| NOV15e | ------------------------------------------------------------ |
| NOV15f | KPKKQYLFKFRATNIMGQFTDSDYSDPVKTLGEGLSERT--------------------- |
| NOV15g | QYYSVYYRNTSGTFMQNFTLHEVTNDFDNMTVSTIIDKLTIFSYYTFWLTASTSVGNGNK |
| NOV15a | FAFARIRQKQKEGGTYSPQDAEIIDTKLKLDQLITVADLELKDERLTRPISKKSFLQHVE |
| NOV15b | SSDIIEVYTDQDVPEGFVGNLTYESISSTAINVSWVPPAQPNGLVFYYVSLILQQTPRHV |
| NOV15c | SLE--------------------------------------------------------- |
| NOV15d | FAFARIRQKQKEGGTYSPQDAEIIDTKLKLDQLITVADLELKDERLTRPISKKSFLQHVE |
| NOV15e | ------------------------------------------------------------ |
| NOV15f | ------------------------------------------------------------ |
| NOV15g | SSDIIEVYTDQDVPEGFVGNLTYESISSTAINVSWVPPAQPNGLVFYYVSLILQQTPRHV |
| NOV15a | ELCTNNNLKFQEEFSELPKFLQDLSSTDADLPWNRAKNRFPNIKPYNNNRVKLIADASVP |
| NOV15b | RPPLVTYERSIYFDNLEKYTDYILKITPSTEKGFSDTYTAQLYIKTEEDVPETSPIINTF |
| NOV15c | ------------------------------------------------------------ |
| NOV15d | ELCTNNNLKFQEEFSELPKFLQDLSSTDADLPWNRAKNRFPNIKPYNNNRVKLIADASVP |
| NOV15e | ------------------------------------------------------------ |
| NOV15f | ------------------------------------------------------------ |
| NOV15g | RPPLVTYERSIYFDNLEKYTDYILKITPSTEKGFSDTYTAQLYIKTEEDVPETSPIINTF |
| NOV15a | GSDYINASYISGYLCPNEFIATQGPLPGTVGDFWRMVWETRAKTLVMLTQCFEKGRIRCH |
| NOV15b | KNLSSTSVLLSWDPPVKPNGAIISYDLTLQGPNENYSFITSDNYIILEELSPFTLYSFFA |
| NOV15c | ------------------------------------------------------------ |
| NOV15d | GSDYINASYISGYLCPNEFIATQGPLPGTVGDFWRMVWETRAKTLVMLTQCFEKGRIRCH |
| NOV15e | ------------------------------------------------------------ |
| NOV15f | ------------------------------------------------------------ |
| NOV15g | KNLSSTSVLLSWDPPVKPNGAIISYDLTLQGPNENYSFITSDNYIILEELSPFTLYSFFA |
| NOV15a | QYWPEDNKPVTVFGDIVITKLMEDVQIDWTIRDLKIERHGDCMTVRQCNFTAWPEHGVPE |
| NOV15b | AARTRKGLGPSSILFFYTDESVPLAPPQNLTLINCTSDFVWLKWSPSPLPGGIVKVYSFK |
| NOV15c | ------------------------------------------------------------ |
| NOV15d | QYWPEDHKPVTVFGDIVITKLMEDVQIDWTIRDLKIERHGDCMTVRQCNFTAWPEHGVPE |
| NOV15e | ------------------------------------------------------------ |
| NOV15f | ------------------------------------------------------------ |
| NOV15g | AARTRKGLGPSSILFFYTDESVPLAPPQNLTLINCTSDFVWLKWSPSPLPGGIVKVYSFK |
| NOV15a | NSAPLIHFVKLVRASRAHDTTPMIVHCSAGVGRTGVFIALDHLTQHINDHDFVDIYGLVA |
| NOV15b | IHEHETDTIYYKNISGFKTEAKLVGLEPVSTYSIRVSAFTKVGNGNQFSNVVKFTTQESV |
| NOV15c | ------------------------------------------------------------ |
| NOV15d | NSAPLIHFVKLVRASRAHDTTPMIVHCSAGVGRTGVFIALDHLTQHINDHDFVDIYGLVA |
| NOV15e | ------------------------------------------------------------ |
| NOV15f | ------------------------------------------------------------ |
| NOV15g | IHEHETDTIYYKNISGFKTEAKLVGLEPVSTYSIRVSAFTKVGNGNQFSNVVKFTTQESV |
| NOV15a | ELRSERMCMVQNLAQYIFLHQCILDLLSNKGSNQPICFVNYSALQKMDSLDAMEGGDVEL |
| NOV15b | PDVVQNMQCMATSWQSVLVKWDPPKKANGIITQYMVTVERNSTKVSPQDHMYTFIKLLAN |
| NOV15c | ------------------------------------------------------------ |
| NOV15d | ELRSERMCMVQNLAQYIFLHQCILDLLSNKGSNQPICFVNYSALQKMDSLDAMEGGDVEL |
| NOV15e | ------------------------------------------------------------ |
| NOV15f | ------------------------------------------------------------ |
| NOV15g | PDVVQNMQCMATSWQSVLVKWDPPKKANGIITQYMVTVERNSTKVSPQDHMYTFIKLLAN |
| NOV15a | EWEETTM----------------------------------------------------- |
| NOV15b | TSYVFKVRASTSAGEGDESTCHVSTLPETVPSVPTNIAFSDVQSTSATLTWIRPDTILGY |
| NOV15c | ------------------------------------------------------------ |
| NOV15d | EWEETTM----------------------------------------------------- |
| NOV15e | ------------------------------------------------------------ |
| NOV15f | ------------------------------------------------------------ |
| NOV15g | TSYVFKVRASTSAGEGDESTCHVSTLPETVPSVPTNIAFSDVQSTSATLTWIRPDTILGY |
| NOV15a | ------------------------------------------------------------ |
| NOV15b | FQNYKITTQLRAQKCKEWESEECVEYQKIQYLYEAHLTEETVYGLKKFRWYRFQVAASTN |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | FQNYKITTQLRAQKCKEWESEECVEYQKIQYLYEAHLTEETVYGLKKFRWYRFQVAASTN |
| NOV15a | ------------------------------------------------------------ |
| NOV15b | AGYGNASNWISTKTLPGPPDGPPENVHVVATSPFSISISWSEPAVITGPTCYLIDVKSVD |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | AGYGNASNWISTKTLPGPPDGPPENVHVVATSPFSISISWSEPAVITGPTCYLIDVKSVD |
| NOV15a | ------------------------------------------------------------ |
| NOV15b | NDEFNISFIKSNEENKTIEIKDLEIFTRYSVVITAFTGNISAAYVEGKSSAEMIVTTLES |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | NDEFNISFIKSNEENKTIEIKDLEIFTRYSVVITAFTGNISAAYVEGKSSAEMIVTTLES |
| NOV15a | ------------------------------------------------------------ |
| NOV15b | APKDPPNNMTFQKIPDEVTKFQLTSLPPSQPNGNIQVYQALVYREDDPTAVQIHNLSIIQ |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | APKDPPNNMTFQKIPDEVTKFQLTSLPPSQPNGNIQVYQALVYREDDPTAVQIHNLSIIQ |
| NOV15a | ------------------------------------------------------------ |
| NOV15b | KTNTFVIAMLEGLKGGHTYNISVYAVNSAGAGPKVPMRITMDIKAPARPKTKPTPIYDAT |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | KTNTFVIAMLEGLKGGHTYNISVYAVNSAGAGPKVPMRITMDIKAPARPKTKPTPIYDAT |
| NOV15a | ------------------------------------------------------------ |
| NOV15b | GKLLVTSTTITIRMPICYYSDDHGPIKNVQVLVTETGAQHDGNVTKWYDAYFNKARPYFT |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | GKLLVTSTTITIRMPICYYSDDHGPIKNVQVLVTETGAQHDGNVTKWYDAYFNKARPYFT |
| NOV15a | ------------------------------------------------------------ |
| NOV15b | NEGFPNPPCTEGKTKFSGNEEIYIIGADNACMIPGNEDKICNGPLKPKKQYLFKFRATNI |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | NEGFPNPPCTEGKTKFSGNEEIYIIGADNACMIPGNEDKICNGPLKPKKQYLFKFRATNI |
| NOV15a | ------------------------------------------------------------ |
| NOV15b | MGQFTDSDYSDPVKTLGEGLSERTLEIILSVTLCILSIILLGTAIFAFARIRQKQKEGGT |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | MGQFTDSDYSDPVKTLGEGLSERTLEIILSVTLCILSIILLGTAIFAFARIRQKQKEGGT |
| NOV15a | ------------------------------------------------------------ |
| NOV15b | YSPQDAEIIDTKLKLDQLITVADLELKDERLTRLLSYRKSIKPISKKSFLQHVEELCTNN |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | YSPQDAEIIDTKLKLDQLITVADLELKDERLTRLLSYRKSIKPISKKSFLQHVEELCTNN |
| NOV15a | ------------------------------------------------------------ |
| NOV15b | NLKFQEEFSELPKFLQDLSSTDADLPWNRAKNRFPNIKPYNNNRVKLIADASVPGSDYIN |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | NLKFQEEFSELPKFLQDLSSTDADLPWNRAKNRFPNIKPYNNNRVKLIADASVPGSDYIN |
| NOV15a | ------------------------------------------------------------ |
| NOV15b | ASYISGYLCPNEFIATQGPLPGTVGDFWRMVWETRAKTLVMLTQCFEKGRIRCHQYWPED |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | ASYISGYLCPNEFIATQGPLPGTVGDFWRMVWETRAKTLVMLTQCFEKGRIRCHQYWPED |
| NOV15a | ------------------------------------------------------------ |
| NOV15b | NKPVTVFGDIVITKLMEDVQIDWTIRDLKIERHGDCMTVRQCNFTAWPEHGVPENSAPLI |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | NKPVTVFGDIVITKLMEDVQIDWTIRDLKIERHGDCMTVRQCNFTAWPEHGVPENSAPLI |
| NOV15a | ------------------------------------------------------------ |
| NOV15b | HFVKLVRASRAHDTTPMIVHCSAGVGRTGVFIALDHLTQHINDHDFVDIYGLVAELRSER |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | HFVKLVRASRAHDTTPMIVHCSAGVGRTGVFIALDHLTQHINDHDFVDIYGLVAELRSER |
| NOV15a | ------------------------------------------------------------ |
| NOV15b | MCMVQNLAQYIFLHQCILDLLSNKGSNQPICFVNYSALQKMDSLDAMEGDVELEWEETTM |
| NOV15c | ------------------------------------------------------------ |
| NOV15d | ------------------------------------------------------------ |
| NOV15e | ------------------------------------------------------------ |
| NOV15f | ------------------------------------------------------------ |
| NOV15g | MCMVQNLAQYIFLHQCILDLLSNKGSNQPICFVNYSALQKMDSLDAMEGDVELEWEETTM |
| NOV15a | (SEQ ID NO: 166) |
| NOV15b | (SEQ ID NO: 168) |
| NOV15c | (SEQ ID NO: 170) |
| NOV15d | (SEQ ID NO: 172) |
| NOV15e | (SEQ ID NO: 174) |
| NOV15f | (SEQ ID NO: 176) |
| NOV15g | (SEQ ID NO: 178) |

Further analysis of the NOV15a protein yielded the following properties shown in Table 15C.

TABLE 15C
Protein Sequence Properties NOV15a

SignalP analysis: Cleavage site between residues 18 and 19
PSORT II analysis:

| | |
|---|---|
| PSG: | a new signal peptide prediction method |
| | N-region: length 2; pos.chg 0; neg.chg 1 |
| | H-region: length 13; peak value 0.00 |
| | PSG score: −4.40 |
| GvH: | von Heijne's method for signal seq. recognition |
| | GvH score (threshold: −2.1): 2.26 |
| | possible cleavage site: between 17 and 18 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 3 | |
| | INTEGRAL | Likelihood = −2.55 Transmembrane 3-19 |
| | INTEGRAL | Likelihood = −1.12 Transmembrane 605-621 |
| | INTEGRAL | Likelihood = −10.46 Transmembrane 1894-1910 |
| | PERIPHERAL | Likelihood = 1.59 (at 2178) |

| | ALOM score: −10.46 (number of TMSs: 3) |
|---|---|
| MTOP: | Prediction of membrane topology (Hartmann et al.) |
| | Center position for calculation: 10 |
| | Charge difference: −1.0 C(−1.0) − N(0.0) |
| | N >= C: N-terminal side will be inside |

>>> membrane topology: type 3a
MITDISC: discrimination of mitochondrial targeting seq

| | | | |
|---|---|---|---|
| R content: | 0 | Hyd Moment(75): | 3.60 |
| Hyd Moment(95): | 6.71 | G content: | 1 |
| D/E content: | 2 | S/T content: | 2 |
| Score: −7.20 | | | |

| | |
|---|---|
| Gavel: | prediction of cleavage sites for mitochondrial preseq |
| | cleavage site motif not found |

NUCDISC: discrimination of nuclear localization signals

pat4: KKPR (4) at 415
pat4: KPKK (4) at 1855
pat7: PLKPKKQ (4) at 1853
bipartite: none
content of basic residues: 8.8%
NLS Score: 0.37

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

56.5%: endoplasmic reticulum
26.1%: mitochondrial
17.4%: nuclear

>> prediction for CG50718-02 is end (k = 23)

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAO18736 | Human NOV2a protein - Homo | 1 . . . 2281 | 2281/2281 (100%) | 0.0 |
| | sapiens, 2281 aa. | 1 . . . 2281 | 2281/2281 (100%) | |
| | [WO200257450-A2, 25-JUL- | | | |
| | 2002] | | | |
| AAG79724 | Human KPP-2, Incyte ID No. | 1 . . . 2281 | 2180/2306 (94%) | 0.0 |
| | 7480588CD1 - Homo sapiens, | 1 . . . 2299 | 2214/2306 (95%) | |
| | 2299 aa. [WO200290530-A2, | | | |
| | 14-NOV-2002] | | | |
| AAO18738 | Human NOV2c protein - Homo | 1 . . . 2281 | 2177/2305 (94%) | 0.0 |
| | sapiens, 2300 aa. | 1 . . . 2300 | 2205/2305 (95%) | |
| | [WO200257450-A2, 25-JUL- | | | |
| | 2002] | | | |
| ABP60057 | Human phosphatase protein | 1 . . . 2281 | 2171/2296 (94%) | 0.0 |
| | SEQ ID #2 - Homo sapiens, | 1 . . . 2291 | 2209/2296 (95%) | |
| | 2291 aa. [WO200279452-A2, | | | |
| | 10-OCT-2002] | | | |
| ABP60058 | Human phosphatase related | 1 . . . 2281 | 1893/2306 (82%) | 0.0 |
| | protein #SEQ ID 4 - Homo | 1 . . . 2301 | 2077/2306 (89%) | |
| | sapiens, 2301 aa. | | | |
| | [WO200279452-A2, 10-OCT- | | | |
| | 2002] | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O88488 | Glomerular mesangial cell | 1 . . . 2281 | 1893/2306 (82%) | 0.0 |
| | receptor protein-tyrosine | 2 . . . 2302 | 2077/2306 (89%) | |
| | phosphatase precursor - Rattus | | | |
| | norvegicus (Rat), 2302 aa. | | | |
| Q8BY76 | Hypothetical fibronectin type | 1 . . . 1042 | 791/1058 (74%) | 0.0 |
| | III domain/fibronectin type III | 1 . . . 1053 | 897/1058 (84%) | |
| | repeat containing protein - Mus | | | |
| | musculus (Mouse), 1086 aa | | | |
| | (fragment). | | | |
| AAO42638 | RE52018p - Drosophila | 1142 . . . 2244 | 311/1133 (27%) | 2e−99 |
| | melanogaster (Fruit fly), 1631 | 497 . . . 1531 | 505/1133 (44%) | |
| | aa. | | | |
| Q8IR87 | CG1817-PC - Drosophila | 1142 . . . 2244 | 311/1133 (27%) | 2e−99 |
| | melanogaster (Fruit fly), 1556 | 497 . . . 1531 | 505/1133 (44%) | |
| | aa. | | | |
| Q9VYW1 | CG1817 protein - Drosophila | 1142 . . . 2244 | 311/1133 (27%) | 2e−99 |
| | melanogaster (Fruit fly), 1962 | 497 . . . 1531 | 505/1133 (44%) | |
| | aa. | | | |

PFam analysis indicates that the NOV15a protein contains the domains shown in the Table 15F.

TABLE 15F
Domain Analysis of NOV15a

| | | Identities/ | |
|---|---|---|---|
| | NOV15a Match | Similarities | Expect |
| Pfam Domain | Region | for the Matched Region | Value |
| fn3 | 54 . . . 141 | 30/91 (33%) | 1.6e−14 |
| | | 63/91 (69%) | |
| fn3 | 301 . . . 383 | 25/88 (28%) | 2.7e−06 |
| | | 59/88 (67%) | |
| fn3 | 561 . . . 643 | 26/86 (30%) | 7.9e−07 |
| | | 56/86 (65%) | |
| fn3 | 657 . . . 738 | 28/87 (32%) | 4.2e−14 |
| | | 62/87 (71%) | |
| fn3 | 751 . . . 834 | 29/87 (33%) | 9.4e−12 |
| | | 67/87 (77%) | |
| fn3 | 846 . . . 926 | 24/87 (28%) | 7.6e−12 |
| | | 62/87 (71%) | |
| fn3 | 938 . . . 1030 | 25/97 (26%) | 6.8e−08 |
| | | 68/97 (70%) | |
| fn3 | 1043 . . . 1124 | 19/87 (22%) | 0.058 |
| | | 56/87 (64%) | |
| fn3 | 1140 . . . 1221 | 28/87 (32%) | 2.4e−09 |
| | | 57/87 (66%) | |
| fn3 | 1233 . . . 1318 | 24/87 (28%) | 8.3e−09 |
| | | 59/87 (68%) | |
| fn3 | 1330 . . . 1408 | 25/86 (29%) | 6.2e−11 |
| | | 58/86 (67%) | |
| fn3 | 1420 . . . 1516 | 29/99 (29%) | 7.7e−09 |
| | | 67/99 (68%) | |
| fn3 | 1528 . . . 1610 | 19/87 (22%) | 2.1e−07 |
| | | 60/87 (69%) | |
| fn3 | 1631 . . . 1724 | 26/97 (27%) | 1e−05 |
| | | 66/97 (68%) | |
| Y_phosphatase | 2008 . . . 2239 | 116/280 (41%) | 5.1e−109 |
| | | 190/280 (68%) | |

### Example 16

The NOV16 clone was analyzed, and the nucleotide and encoded polypeptide sequences are shown in Table 16A.

TABLE 16A
NOV16 Sequence Analysis

| | | |
|---|---|---|
| NOV16a, CG50934-03 | SEQ ID NO: 179 | 964 bp |

| | | |
|---|---|---|
| DNA Sequence | ORF Start: ATG at 67 | ORF Stop: TGA at 655 |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

NOV16a, CG50934-03

| | | | |
|---|---|---|---|
| Protein Sequence | SEQ ID NO: 180 | 196 aa | MW at 21256.7kD |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

| | | |
|---|---|---|
| NOV16b, CG50934-01 | SEQ ID NO: 181 | 1171 bp |

| | | |
|---|---|---|
| DNA Sequence | ORF Start: at 187 | ORF Stop: TGA at 805 |

ATGCTGTGGCTACTGCTCCTGACCCTCCCCTGCCTGATGGGCTCTGTGCCCAGGAACCCAGGCGAGTC
CGCCCCACCCAATGCCCCTGCTGCCCAGGACCCCCTCCTTGCCCTGCCCCGGGCTCAGAGTGCCAGCC
CTGGGGTGGGTGGGGACCATCTGATTGCCGGTCTCTCCTGGTGCCCCTGAGCTCTGGGAAGACCCTCG
TCCGTCCCCCTCATGAGCCCGGCACGGGGCGTGAGCTGGTGGGCATCACTGGGGGCTGCGACGTCTCG
GCCAGGAGGCACCCCTGGCAGGGAGGAGTTGGAGGCTTGCGCGTTTAGAGTGCAGGTGGGGCAGCTGA
GGCTCTATGAGGACGACCAGCGGACGAAGGTGGTTGAGATCGTCCGTCACCCCCAGTACAACGAGAGC
CTGTCTGCCCAGGGCGGTGCGGACATCGCCCTGCTGAAGCTGGAGGCCCCGGTGCCGCTGTCTGAGCT
CATCCACCCGGTCTCGCTCCCGTCTGCCTCCCTGGACGTGCCCTCGGGGAAGACCTGCTGGGTGACCG
GCTGGGGTGTCATTGGACGTGGAGGTCAGGAGCAGGACCACTTGGGTGGGATGTGGAGAGATGACCCG
GAATGTCGGTGCAGGCCTGGGCTCCAGACGCGTCCTGGATGGCTTCCTGCCGCTGCCGAGACGGATGG
CCACAGGCCAGGTGGCTCAGAGCAGCAGGAATGTACTATCTCACGGCTCTGCAGGCGGAACGTCCACA
CTCCAGGCGTGGGCAGGAACTACTGCCCTGGCCCCTCAGCTTGTGGGAGGCGACGGTGAAGGTCAGGA
GCAACGTCCTCTGTAACCAGACCTGTCGCCGCCGCTTTCCTTCCAACCACACTGAGCGGTTTGAGCGG
CTCATCAAGGACGACATGCTGTGTGCCGGGGACGGGAACCACGGCTCCTGGCCAGGCGACAACGGGGG
CCCCCTCCTGTGCAGGCGGAATTGCACCTGGGTCCAGGTGGAGGTGGTGAGCTGGGGCAAACTCTGCG
GCCTTCGCGGCTATCCCGGCATGTACACCCGCGTGACGAGCTACGTGTCCTGGATCCGCCAGTACGTC
CCGCCGTTCCCCAGACGCTAGCTGGGGTGCAGTGGGGTCTGCATGATCCAGGAGGGCCCGTCTTCCTT
GTGGACACGCCTGCT

NOV1 6b, CG50934-01

| | | | |
|---|---|---|---|
| Protein Sequence | SEQ ID NO: 182 | 206 aa | MW at 22191.8kD |

ALGRPSSVPLMSPARGVSWWASLGAATSRPGGTPGREELEACAFRVQVGQLRLYEDDQRTKVVEIVRH
PQYNESLSAQGGADIALLKLEAPVPLSELIHPVSLPSASLDVPSGKTCWVTGWGVIGRGGQEQDHLGG
MWRDDPECRCRPGLQTRPGWLPAAAETDGHRPGGSEQQECTISRLCRRNVHTPGVGRNYCPGPSACGR
RR

| | | |
|---|---|---|
| NOV16c, CG50934-02 | SEQ ID NO: 183 | 843 bp |

| | | |
|---|---|---|
| DNA Sequence | ORF Start: ATG at 26 | ORF Stop: TAG at 836 |

GAGGTGGAGGTTGCAGTAAGCCAAGATGGCGCCACTGCACTCTAGCCTGTTTCTGCTGAGCGGGACCA
TGAGCCCAAAAGTGGGCATCACTGGGGGCTGCGACGTCTCGGCCAGGAGGCACCCCTGGCAGGTCAGC
CTGAGGTTCTACAGCATGAAGAAGGGTCTGTGGGAGCCCATCTGTGGGGGCTCCCTCATCCACCCAGA
GTGGGTGCTGACCGCCGCCCACTGCGTCGAGCTTGAGGAGTTGGAGGCTTGCGCGTTTAGAGTGCAGG
TGGGGCAGCTGAGGCTCTATGAGGACGACCAGCGGACGAAGGTGGTTGAGATCGTCCGTCACCCCCAG
TACAACGAGAGCCTGTCTGCCCAGGGCGGTGCGGACATCGCCCTGCTGAAGCTGGAGGCCCCGGTGCC
GCTGTCTGAGCTCATCCACCCGGTCTCGCTCCCGTCTGCCTCCCTGGACGTGCCCTCGGGGAAGACCT
GCTGGGTGACCGGCTGGGGTGTCATTTGGGGACACGTTTTCCTGCTCCCGCCACCCCACCTCAGGGCA
GCGGAAGGTCCAATCATGAGGACCCGAGCTTGCGAGAGGATGTATCACAAAGGCCCCACTGCCCACGT
CACCATCATCAAGGCTGCCATGCCGTGTGCAGGGGCTGAGCGCCATCTCTCCCCACAGGGCGACAACG
GGGGCCCCCTCCTGTGCAGGCGGAATTGCACCTGGGTCCAGGTGGAGGTGGTGAGCTGGGGCAAACTC
TGCGGCCTTCGCTATCCCGGCATGTACACCCGCGTGACGAGCTACGTGTCCTGGATCCGCCAGTACGT
CCCGCCGTTCCCCAGACGCTAGCTGGG

NOV16c, CG50934-02

| | | | |
|---|---|---|---|
| Protein Sequence | SEQ ID NO: 184 | 270 aa | MW at 29993.6kD |

MAPLHSSLFLLSGTMSPKVGITGGCDVSARRHPWQVSLRFYSMKKGLWEPICGGSLIHPEWVLTAAHC
VELEELEACAFRVQVGQLRLYEDDQRTKVVEIVRHPQYNESLSAQGGADIALLKLEAPVPLSELIHPV
SLPSASLDVPSGKTCWVTGWGVIWGHVFLLPPPHLRAAEGPIMRTRACERNYHKGPTAHVTIIKAAMP
CAGAERHLSPQGDNGGPLLCRRNCTWVQVEVVSWGKLCGLRYPGMYTRVTSYVSWIRQYVPPFPRR

| | |
|---|---|
| SEQ ID NO: 185 | 964 bp |

| | | |
|---|---|---|
| NOV16d, SNP13381559 of | ORF Start: ATG at 67 | ORF Stop: TGA at 655 |

| | | |
|---|---|---|
| CG50934-03, DNA Sequence | SNP Pos: 338 | SNP Change: T to C |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGCCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

| | | |
|---|---|---|
| NOV16d, SNP13381559 of | SEQ ID NO: 186 | MW at 21228.6kD |

| | | | |
|---|---|---|---|
| CG50934-03, Protein Sequence | SNP Pos: 91 | 196 aa | SNP Change: Val to Ala |

MSPARGVSWWASLGAATSRPGGTPGREELEACAFRVQVGQLRLYEDDQRTKVVEIVRHPQYNESLSAQ
GGADIALLKLEAPVPLSELIHPASLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

| | |
|---|---|
| SEQ ID NO: 187 | 964 bp |

| | | |
|---|---|---|
| NOV16e, SNP13381558 of | ORF Start: ATG at 67 | ORF Stop: TGA at 655 |

| | | |
|---|---|---|
| CG50934-03, DNA Sequence | SNP Pos: 359 | SNP Change: G to T |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCTGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

| | | |
|---|---|---|
| NOV16e, SNP13381558 of | SEQ ID NO: 188 | MW at 21213.7kD |

| | | | |
|---|---|---|---|
| CG50934-03, Protein Sequence | SNP Pos: 98 | 196 aa | SNP Change: Arg to Leu |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASLDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

| | |
|---|---|
| SEQ ID NO: 189 | 964 bp |

| | | |
|---|---|---|
| NOV16f, SNP13376399 of | ORF Start: ATG at 67 | ORF Stop: TGA at 655 |

| | | |
|---|---|---|
| CG50934-03, DNA Sequence | SNP Pos: 641 | SNP Change: G to A |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTATGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

| | | |
|---|---|---|
| NOV16f, SNP13376399 of | SEQ ID NO: 190 | MW at 21316.7kD |

| | | | |
|---|---|---|---|
| CG50934-03, Protein Sequence | SNP Pos: 192 | 196 aa | SNP Change: Cys to Tyr |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSAYGRRR

| | |
|---|---|
| SEQ ID NO: 191 | 964 bp |

| | | |
|---|---|---|
| NOV16g, SNP13378301 of | ORF Start: ATG at 67 | ORF Stop: TGA at 655 |

| | | |
|---|---|---|
| CG50934-03, DNA Sequence | SNP Pos: 868 | SNP Change: G to A |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTACGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

| | |
|---|---|
| NOV16g, SNP13378301 of | MW at 21256.7kD |

| | | | |
|---|---|---|---|
| CG50934-03, Protein Sequence | SEQ ID NO: 192 | 196 aa | SNP Change: no change |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

| | |
|---|---|
| SEQ ID NO: 193 | 964 bp |

| | | |
|---|---|---|
| NOV16h, SNP13378300 of | ORF Start: ATG at 67 | ORF Stop: TGA at 655 |

| | | |
|---|---|---|
| CG50934-03, DNA Sequence | SNP Pos: 907 | SNP Change: C to T |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGATGAGCTACGTGTCCTGGATCCGCCAGTACGTCCCGCCGTTCCCCAG
ACGCTAGCTGGG

| | |
|---|---|
| NOV16h, SNP13378300 of | MW at 21256.7kD |

| | | | |
|---|---|---|---|
| CG50934-03, Protein Sequence | SEQ ID NO: 194 | 196 aa | SNP Change: no change |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

A ClustalW comparison of the above protein sequences yields the following sequence alignment shown in Table 16B.

TABLE 16B
Comparison of the NOV16 protein sequences.

| | |
|---|---|
| NOV16a | --------------MSPARGVS-----------WWASLGAATSRP-------GGTPGR-- |
| NOV16b | ----ALGRPSSVPLMSPARGVS-----------WWASLGAATSRP-------GGTPGR-- |
| NOV16c | MAPLHSSLFLLSGTMSPKVGITGGCDVSARRHPWQVSLRFYSMKKGLWEPICGGSLIHPE |
| NOV16a | -----------EELEACAFRVQVGQLRLYEDDQRTKVVEIVRHPQYNESLSAQGGADIAL |
| NOV16b | -----------EELEACAFRVQVGQLRLYEDDQRTKVVEIVRHPQYNESLSAQGGADIAL |
| NOV16c | WVLTAAHCVELEELEACAFRVQVGQLRLYEDDQRTKVVEIVRHPQYNESLSAQGGADIAL |
| NOV16a | LKLEAPVPLSELIHPVSLPSASRDVPSGKTCWVTGWGVIG------RGGQEQDHLGGMWR |
| NOV16b | LKLEAPVPLSELIHPVSLPSASLDVPSGKTCWVTGWGVIG------RGGQEQDHLGGMWR |
| NOV16c | LKLEAPVPLSELIHPVSLPSASLDVPSGKTCWVTGWGVIWGHVFLLPPPHLRAAEGPIMR |
| NOV16a | D-DPECRCRPGLQTRPGWLPAAAETDGHRPGGSEQQECTISRLCRRNVHTPGVGRNYCPG |
| NOV16b | D-DPECRCRPGLQTRPGWLPAAAETDGHRPGGSEQQECTISRLCRRNVHTPGVGRNYCPG |
| NOV16c | TRACERMYHKGPTAHVTIIKAAMPCAGAERHLSPQGDNGGPLLCRRN--CTWVQVEVVSW |
| NOV16a | PSACGRRR------------------------ |
| NOV16b | PSACGRRR------------------------ |
| NOV16c | GKLCGLRYPGMYTRVTSYVSWIRQYVPPFPRR |
| NOV16a | (SEQ ID NO: 180) |
| NOV16b | (SEQ ID NO: 182) |
| NOV16c | (SEQ ID NO: 184) |

Further analysis of the NOV16a protein yielded the following properties shown in Table 16C.

TABLE 16C
Protein Sequence Properties NOV16a

SignalP analysis: Cleavage site between residues 19 and 20
PSORT II analysis:

| | |
|---|---|
| PSG: | a new signal peptide prediction method |
| | N-region: length 5; pos.chg 1; neg.chg 0 |
| | H-region: length 13; peak value 6.79 |
| | PSG score: 2.39 |
| GvH: | von Heijne's method for signal seq. recognition |
| | GvH score (threshold: −2.1): −3.86 |
| | possible cleavage site: between 18 and 19 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) .. fixed | |
| | PERIPHERAL | Likelihood = 4.88 (at 73) |

| | ALOM score: 4.88 (number of TMSs: 0) |
|---|---|
| MTOP: | Prediction of membrane topology (Hartmann et al.) |
| | Center position for calculation: 6 |
| | Charge difference: −3.0 C(−1.0) − N(2.0) |
| | N >= C: N-terminal side will be inside |

MITDISC: discrimination of mitochondrial targeting seq

| | | | |
|---|---|---|---|
| R content: | 3 | Hyd Moment(75): | 7.01 |
| Hyd Moment(95): | 6.08 | G content: | 5 |
| DIE content: | 1 | S/T content: | 6 |
| Score: −2.71 | | | |

| | |
|---|---|
| Gavel: | prediction of cleavage sites for mitochondrial preseq |
| | R-2 motif at 29 SRP\|GG |

NUCDISC: discrimination of nuclear localization signals

pat4: none
pat7: none
bipartite: none
content of basic residues: 12.2%
NLS Score: −0.47

KDEL: ER retention motif in the C-terminus: none
ER Membrane Retention Signals:

XXRR-like motif in the N-terminus: SPAR

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 70.6 |
| COIL: | Lupas's algorithm to detect coiled-coil regions |
| | total: 0 residues |

Final Results (k = 9/23):

55.6%: extracellular, including cell wall
33.3%: mitochondrial
11.1%: cytoplasmic

>> prediction for CG50934-03 is exc (k = 9)

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE08591 | Human NOV12 protein - Homo | 12 . . . 117 | 106/106 (100%) | 2e−56 |
| | sapiens, 220 aa. [WO200161009- | 1 . . . 106 | 106/106 (100%) | |
| | A2, 23-AUG-2001] | | | |
| AAU82736 | Amino acid sequence of novel | 5 . . . 117 | 97/121 (80%) | 3e−47 |
| | human protease #35 - Homo | 184 . . . 302 | 101/121 (83%) | |
| | sapiens, 948 aa. [WO200200860- | | | |
| | A2, 03-JAN-2002] | | | |
| AAE14347 | Human protease PRTS-12 | 5 . . . 117 | 97/121 (80%) | 3e−47 |
| | protein - Homo sapiens, 262 aa. | 30 . . . 148 | 101/121 (83%) | |
| | [WO2001 83775-A2, 08-NOV- | | | |
| | 2001] | | | |
| AAE08587 | Human NOV8 protein - Homo | 25 . . . 117 | 91/93 (97%) | 6e−47 |
| | sapiens, 290 aa. [WO200161009- | 84 . . . 176 | 91/93 (97%) | |
| | A2, 23-AUG-2001] | | | |
| AAE08590 | Human NOV11 protein - Homo | 27 . . . 117 | 90/91 (98%) | 2e−46 |
| | sapiens, 285 aa. [WO200161009- | 81 . . . 171 | 90/91 (98%) | |
| | A2, 23-AUG-2001] | | | |

In a BLAST search of public sequence databases, the NOV16a protein was found to have homology to the proteins shown in the BLASTP data in Table 16E.

TABLE 16E
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAP21675 | Mast cell protease-11 - Mus | 10 . . . 114 | 63/105 (60%) | 3e-27 |
| | musculus (Mouse), 318 aa. | 75 . . . 173 | 77/105 (73%) | |
| AAA30855 | Mastin precursor - Canis sp, 251 | 27 . . . 114 | 55/88 (62%) | 5e-22 |
| | aa (fragment). | 53 . . . 140 | 61/88 (68%) | |
| P19236 | Mastocytoma protease precursor | 27 . . . 114 | 55/88 (62%) | 5e-22 |
| | (EC 3.4.21.-) - Canis familiaris | 71 . . . 158 | 61/88 (68%) | |
| | (Dog), 269 aa. | | | |
| Q8SQ44 | Tryptase precursor - Sus scrofa | 35 . . . 114 | 49/80 (61%) | 1e-18 |
| | (Pig), 277 aa. | 90 . . . 167 | 60/80 (74%) | |
| I48685 | mast cell proteinase 6 (EC | 34 . . . 114 | 44/81 (54%) | 2e-16 |
| | 3.4.21.-) precursor - mouse, 230 | 87 . . . 164 | 54/81 (66%) | |
| | aa. | | | |

PFam analysis indicates that the NOV16a protein contains the domains shown in the Table 16F.

TABLE 16F
Domain Analysis of NOV16a

| | | Identities/ | |
|---|---|---|---|
| | | Similarities | |
| Pfam | NOV16a Match | for the | Expect |
| Domain | Region | Matched Region | Value |
| trypsin | 19 . . . 172 | 52/266 (20%) | 0.00069 |
| | | 108/266 (41%) | |

### Example 17

The NOV17 clone was analyzed, and the nucleotide and encoded polypeptide sequences are shown in Table 17A.

TABLE 17A
NOV17 Sequence Analysis

| | | |
|---|---|---|
| NOV17a, CG51213-04 | SEQ ID NO: 195 | 2804 bp |
| DNA Sequence | ORF Start: ATG at 71 | ORF Stop: TAG at 2636 |

TGGCCAGCCAGGCCTGAAGCGATCGGTCAGCCGAGAGCGCTACGTGGAGACCCTGGTGGTGGCTGACA
AGATGATGGTGGCCTATCACGGGCGCCGGGATGTGGAGCAGTATGTCCTGGCCATCATGAACATTCAG
GTTGCCAAACTTTTCCAGGACTCGAGTCTGGGAAGCACCGTTAACATCCTCGTAACTCGCCTCATCCT
GCTCACGGAGGACCAGCCCACTCTGGAGATCACCCACCATGCCGGGAAGTCCCTGGACAGCTTCTGTA
AGTGGCAGAAATCCATCGTGAACCACAGCGGCCATGGCAATGCCATTCCAGAGAACGGTGTGGCTAAC
CATGACACAGCAGTGCTCATCACACGCTATGACATCTGCATCTACAAGAACAAACCCTGCGGCACACT
AGGCCTGGCCCCGGTGGGCGGAATGTGTGAGCGCGAGAGAAGCTGCAGCGTCAATGAGGACATTGGCC
TGGCCACAGCGTTCACCATTGCCCACGAGATCGGGCACACATTCGGCATGAACCATGACGGCGTGGGA
AACAGCTGTGGGGCCCGTGGTCAGGACCCAGCCAAGCTCATGGCTGCCCACATTACCATGAAGACCAA
CCCATTCGTGTGGTCATCCTGCAGCCGTGACTACATCACCAGCTTTCTAGACTCGGGCCTGGGGCTCT
GCCTGAACAACCGGCCCCCCAGACAGGACTTTGTGTACCCGACAGTGGCACCGGGCCAAGCCTACGAT
GCAGATGAGCAATGCCGCTTTCAGCATGGAGTCAAATCGCGTCAGTGTAAATACGGGGAGGTCTGCAG
CGAGCTGTGGTGTCTGAGCAAGAGCAACCGGTGCATCACCAACAGCATCCCGGCCGCCGAGGGCACGC
TGTGCCAGACGCACACCATCGACAAGGGGTGGTGCTACAAACGGGTCTGTGTCCCCTTTGGGTCGCGC
CCAGAGGGTGTGGACGGAGCCTGGGGGCCGTGGACTCCATGGGGCGACTGCAGCCGGACCTGTGGCGG
CGGCGTGTCCTCTTCTAGCCGTCACTGCGACAGCCCCAGGCCAACCATCGGGGGCAAGTACTGTCTGA
GTGAGAGAAGGCGGCACCGCTCCTGCAACACGGATGACTGTCCCCCTGGCTCCCAGGACTTCAGAGAA
GTGCAGTGTTCTGAATTTGACAGCATCCCTTTCCGTGGGAAATTCTACAAGTGGAAAACGTACCGGGG
AGGGGGCGTGAAGGCCTGCTCGCTCACGAGCCTAGCGGAAGGCTTCAACTTCTACACGGAGAGGGCGG
CAGCCGTGGTGGACGGGACACCCTGCCGTCCAGACACGGTGGACATTTGCGTCAGTGGCGAATGCAAG
CACGTGGGCTGCGACCGAGTCCTGGGCTCCGACCTGCGGGAGGACAAGTGCCGAGTGTGTGGCGGTGA
CGGCAGTGCCTGCGAGACCATCGAGGGCGTCTTCAGCCCAGCCTCACCTGGGGCCGGGTACGAGGATG
TCGTCTGGATTCCCAAAGGCTCCGTCCACATCTTCATCCAGGATCTGAACCTCTCTCTCAGTCACTTG
GCCCTGAAGGGAGACCAGGAGTCCCTGCTGCTGGAGGGGCTGCCTGGGACCCCCCAGCCCCACCGTCT
GCCTCTAGCTGGGACCACCTTTCAACTGCGACAGGGGCCAGACCAGGTCCAGAGCCTCGAAGCCCTGG
GACCGATTAATGCATCTCTCATCGTCATGGTGCTGGCCCGGACCGAGCTGCCTGCCCTCCGCTACCGC
TTCAATGCCCCCATCGCCCGTGACTCGCTGCCCCCCTACTCCTGGCACTATGCGCCCTGGACCAAGTG
CTCGGCCCAGTGTGCAGGCGGTAGCCAGGTGCAGGCGGTGGAGTGCCGCAACCAGCTGGACAGCTCCG
CGGTCGCCCCCCACTACTGCAGTGCCCACAGCAAGCTGCCCAAAAGGCAGCGCGCCTGCAACACGGAG
CCTTGCCCTCCAGACTGGGTTGTAGGGAACTGGTCGCTCTGCAGCCGCAGCTGCGATGCAGGCGTGCG
CAGTCGCTCGGTCGTGTGCCAGCGCCGCGTCTCTGCCGCGGAGGAGAAGGCGCTGGACGACAGCGCAT
GCCCGCAGCCGCGCCCACCTGTACTGGAGGCCTGCCACGGCCCCACTTGCCCTCCGGAGTGGGCGGCC
CTCGACTGGTCTGAGTGCACCCCCAGCTGCGGGCCGGGCCTCCGCCACCGCGTGGTCCTTTGCAAGAG
CGCAGACCACCGCGCCACGCTGCCCCCGGCGCACTGCTCACCCGCCGCCAAGCCACCGGCCACCATGC
GCTGCAACTTGCGCCGCTGCCCCCCGGCCCGCTGGGTGGCTGGCGAGTGGGGTGAGTGCTCTGCACAG
TGCGGCGTCGGGCAGCGGCAGCGCTCGGTGCGCTGCACCAGCCACACGGGCCAGGCGTCGCACGAGTG
CACGGAGGCCCTGCGGCCGCCCACCACGCAGCAGTGTGAGGCCAAGTGCGACAGCCCAACCCCCGGGG
ACGGCCCTGAAGAGTGCAAGGATGTGAACAAGGTCGCCTACTGCCCCCTGGTGCTCAAATTTCAGTTC
TGCAGCCGAGCCTACTTCCGCCAGATGTGCTGCAAAACCTGCCAGGGCCACTAGGGGGCGCGCGGCAC
CCGGAGCCACAGCTGGCGGGGTCTCCGCCGCCAGCCCTGCAGCGGGCCGGCCAAAGGGGGCCCCGGGG
GGGCGGGAACTGGGAGGGAAGGGTGAGACGGAGCCGGAAGTTATTTATTGGGAACCCCTGCAGGGCCC
TGGCTGGGGGGATGGA

| | | | |
|---|---|---|---|
| NOV17a, CG51213-04 | SEQ ID NO: 196 | 855 aa | MW at 93285.7kD |
| Protein Sequence | | | |

MMVAYHGRRDVEQYVLAINNIQVAKLFQDSSLGSTVNILVTRLILLTEDQPTLEITHHAGKSLDSFCK
WQKSIVNHSGHGNAIPENGVANHDTAVLITRYDICIYKNKPCGTLGLAPVGGMCERERSCSVNEDIGL
ATAFTIAHEIGHTFGMNHDGVGNSCGARGQDPAKLMAAHITMKTNPFVWSSCSRDYITSFLDSGLGLC
LNNRPPRQDFVYPTVAPGQAYDADEQCRFQNGVKSRQCKYGEVCSELWCLSKSNRCITNSIPAAEGTL
CQTHTIDKGWCYKRVCVPFGSRPEGVDGAWGPWTPWGDCSRTCGGGVSSSSRHCDSPRPTIGGKYCLS
ERRRHRSCNTDDCPPGSQDFREVQCSEFDSIPFRGKFYKWKTYRGGGVKACSLTSLAEGFNFYTERAA
AVVDGTPCRPDTVDICVSGECKHVGCDRVLGSDLREDKCRVCGGDGSACETIEGVFSPASPGAGYEDV
VWIPKGSVHIFIQDLNLSLSHLALKGDQESLLLEGLPGTPQPHRLPLAGTTFQLRQGPDQVQSLEALG
PINASLIVMVLARTELPALRYRFNAPIARDSLPPYSWHYAPWTKCSAQCAGGSQVQAVECRNQLDSSA
VAPHYCSAHSKLPKRQRACNTEPCPPDWVVGNWSLCSRSCDAGVRSRSVVCQRRVSAAEEKALDDSAC
PQPRPPVLEACHGPTCPPEWAALDWSECTPSCGPGLRHRVVLCKSADHRATLPPAHCSPAAKPPATMR
CNLRRCPPARWVAGEWGECSAQCGVGQRQRSVRCTSHTGQASHECTEALRPPTTQQCEAKCDSPTPGD
GPEECKDVNKVAYCPLVLKFQFCSRAYFRQMCCKTCQGH

| | | |
|---|---|---|
| NOV17b, CG51213-01 | SEQ ID NO: 197 | 2266 bp |
| DNA Sequence | ORF Start: ATG at 589 | ORF Stop: TAG at 2158 |

CTCGGGCCTGGGGCTCTGCCTGAACAACCGGCCCCCCAGACAGGACTTTGTGTACCCGACAGTGGCAC
CGGGCCAAGCCTACGATGCAGATGAGCAATGCCGCTTTCAGCATGGAGTCAAATCGCGTCAGTTGGTG
CTACAAACGGGTCTGTGTCCCCTTTGGGTCGCGCCCAGAGGGTGTGGACGGAGCCTGGGGGCCGTGGA
CTCCATGGGGCGACTGCAGCCGGACCTGTGGCGGCGGCGTGTCCTCTTCTAGCCGTCACTGCGACAGC
CCCAGGCCAACCATCGGGGGCAAGTACTGTCTGGGTGAGAGAAGGCGGCACCGCTCCTGCAACACGGA
TGACTGTCCCCCTGGCTCCCAGGACTTCAGAGAAGTGCAGTGTTCTGAATTTGACAGCATCCCTTTCC
GTGGGAAATTCTACAAGTGGAAAACGTACCGGGGAAGGTGAGTGTGGGACTCCAAAGGCTGTGGGGCC
GTGAAGGGCAGCCGTGGGAGTGTCCAGCAGCAGGTGGATGAATGCAGCATCCCGGGGTCTGCCATGAG
CCCTGTCCCCACCCGGGGAGACAGAGTACCTGGGATACGGTACCATGGGGGTTCAACGTGACGCTGGG
AGCCCCCACTCCCTCTGCCCAAGCTGCCCTTCCTCTTGGGTCTGGGGTCTGTCCCTCTTGGCCTCACT
CCCCCAGGGAGCAAGCAAAGAGTTCCGGGGTGGCCTGGCCCGTGGTGTGACGGGGCCGTGCCCCCCAG
GGGGCGTGAAGGCCTGCTCGCTCACGTGCCTAGCGGAAGGCTTCAACTTCTACACGGAGAGGGCGGCA
GCCGTGGTGGACGGGACACCCTGCCGTCCAGACACGGTGGACATTTGCGTCAGTGGCGAATGCAAGCA
CGTGGGCTGCGACCGAGTCCTGGGCTCCGACCTGCGGGAGGACAAGTGCCGAGTGTGTGGCGGTGACG
GCAGTGCCTGCGAGACCATCGAGGGCGTCTTCAGCCCAGCCTCACCTGGGGCCGGGTACGAGGATGTC
GTCTGGATTCCCAAAGGCTCCGTCCACATCTTCATCCAGGATCTGAACCTCTCTCTCAGTCACTTGGC
CCTGAAGGGAGACCAGGAGTCCCTGCTGCTGGAGGGGCTGCCTGGGACCCCCCAGCCCCACCGTCTGC
CTCTAGCTGGGACCACCTTTCAACTGCGACAGGGGCCAGACCAGGTCCAGAGCCTCGAAGCCCTGGGA
CCGATTAATGCATCTCTCATCGTCATGGTGCTGGCCCGGACCGAGCTGCCTGCCCTCCGCTACCGCTT
CAATGCCCCCATCGCCCGTGACTCGCTGCCCCCCTACTCCTGGCACTATGCGCCCTGGACCAAGTGCT
CGGCCCAGTGTGCAGGCGGTAGCCAGGTGCAGGCGGTGGAGTGCCGCAACCAGCTGGACAGCTCCGCG
GTCGCCCCCCACTACTGCAGTGCCCACAGCAAGCTGCCCAAAAGGCAGCGCGCCTGCAACACGGAGCC
TTGCCCTCCAGACTGGGTTGTAGGGAACTGGTCGCTCTGCAGCCGCAGCTGCGATGCAGGCGTGCGCA
GCCGCTCGGTCGTGTGCCAGCGCCGCGTCTCTGCCGCGGAGGAGAAGGCGCTGGACGACAGCGCATGC
CCGCAGCCGCGCCCACCTGTACTGGAGGCCTGCCACGGCCCCACTTGCCCTCCGGAGTGGGCGGCCCT
CGACTGGTCTGAGTGCACCCCCAGCTGCGGGCCGGGCCTCCGCCACCGCGTGGTCCTTTGCAAGAGCG
CAGACCACCGCGCCACGCTGCCCCCGGCGCACTGCTCACCCGCCGCCAAGCCACCGGCCACCATGCGC
TGCAACTTGCGCCGCTGCCCCCCGGCCCGCTGGGTGGCTGGCGAGTGGGGTGAGTGCTCTGCACAGTG
CGGCGTCGGGCAGCGGCAGCGCTCGGTGCGCTGCACCAGCCACACGGGCCAGGCGTCGCACGAGTGCA
CGGAGGCCCTGCGGCCGCCCACCACGCAGCAGTGTGAGGCCAAGTGCGACAGCCCAACCCCCGGGGAC
GGCCCTGAAGAGTGCAAGGATGTGAACAAGGTCGCCTACTGCCCCCTGGTGCTCAAATTTCAGTTCTG
CAGCCGAGCCTACTTCCGCCAGATGTGCTGCAAAACCTGCCAGGGCCACTAGGGGGCGCGCGGCACCC
GGAGCCACAGCTGGCGGGGTCTCCGCCGCCAGCCCTGCAGCTGGGCCGGCCAGAGGGGGCCCCGGGGG
GGCGGGAACTGGGAGGGAAGGG

| | | | |
|---|---|---|---|
| NOV17b, CG51213-01 | SEQ ID NO: 198 | 523 aa | MW at 56126.2kD |
| Protein Sequence | | | |

MGVQRDAGSPHSLCPSCPSSWVWGLSLLASLPQGASKEFRGGLARGVTGPCPPGGVKACSLTCLAEGF
NFYTERAAAVVDGTPCRPDTVDICVSGECKHVGCDRVLGSDLREDKCRVCGGDGSACETIEGVFSPAS
PGAGYEDVVWIPKGSVHIFIQDLNLSLSHLALKGDQESLLLEGLPGTPQPHRLPLAGTTFQLRQGPDQ
VQSLEALGPINASLIVMVLARTELPALRYRFNAPIARDSLPPYSWHYAPWTKCSAQCAGGSQVQAVEC
RNQLDSSAVAPHYCSAHSKLPKRQRACNTEPCPPDWVVGNWSLCSRSCDAGVRSRSVVCQRRVSAAEE
KALDDSACPQPRPPVLEACHGPTCPPEWAALDWSECTPSCGPGLRHRVVLCKSADHRATLPPAHCSPA
AKPPATMRCNLRRCPPARWVAGEWGECSAQCGVGQRQRSVRCTSHTGQASNECTEALRPPTTQQCEAK
CDSPTPGDGPEECKDVNKVAYCPLVLKFQFCSRAYFRQMCCKTCQGH

| | | |
|---|---|---|
| NOV17c, 306345264 | SEQ ID NO: 199 | 706 bp |
| DNA Sequence | ORF Start: at 2 | ORF Stop: end of sequence |

CACCGGATCCTGGCACTATGCGCCCTGGACCAAGTGCTCGGCCCAGTGTGCAGGCGGTAGCCAGGTGC
AGGCGGTGGAGTGCCGCAACCAGCTGGACAGCTCCGCGGTCGCCCCCCACTACTGCAGTGCCCACAGC
AAGCTGCCCAAAAGGCAGCGCGCCTGCAACACGGAGCCTTGCCCTCCAGACTGGGTTGTAGGGAACTG
GTCGCTCTGCAGCCGCAGCTGCGATGCAGGCGTGCGCAGCCGCTCGGTCGTGTGCCAGCGCCGCGTCT
CTGCCGCGGAGGAGAAGGCGCTGGACGACAGCGCATGCCCGCAGCCGCGCCCACCTGTACTGGAGGCC
TGCCACGGCCCCACTTGCCCTCCGGAGTGGGCGGCCCTCGACTGGTCTGAGTGCACCCCCAGCTGCGG
GCCGGGCCTCCGCCACCGCGTGGTCCTTTGCAAGAGCGCAGACCACCGCGCCACGCTGCCCCCGGCGC
ACTGCTCACCCGCCGCCAAGCCACCGGCCACCATGCGCTGCAACTTGCGCCGCTGCCCCCCGGCCCGC
TGGGTGGCTGGCGAGTGGGGTGAGTGCTCTGCACAGTGCGGCGTCGGGCAGCGGCAGCGCTCGGTGCG
CTGCACCAGCCACACGGGCCAGGCGTCGCACGAGTGCACGGAGGCCCTGCGGCCGCCCACCACGCAGC
AGTGTGAGGCCAAGTGCCTCGAGGGC

| | | | |
|---|---|---|---|
| NOV17c, 306345264 | SEQ ID NO: 200 | 235 aa | MW at 25395.4kD |
| Protein Sequence | | | |

TGSWHYAPWTKCSAQCAGGSQVQAVECRNQLDSSAVAPHYCSAHSKLPKRQRACNTEPCPPDWVVGNW
SLCSRSCDAGVRSRSVVCQRRVSAAEEKALDDSACPQPRPPVLEACHGPTCPPEWAALDWSECTPSCG
PGLRHRVVLCKSADHRATLPPAHCSPAAKPPATMRCNLRRCPPARWVAGEWGECSAQCGVGQRQRSVR
CTSHTGQASHECTEALRPPTTQQCEAKCLEG

| | | |
|---|---|---|
| NOV17d, CG51213-02 | SEQ ID NO: 201 | 3199 bp |
| DNA Sequence | ORF Start: at 1297 | ORF Stop: at 3199 |

TAAAGGGTTCAGCCTGGTGCCTGGTCCAGAGATAGTGGTGGTCATTGTTACCCCATAATGGCATTGGT
GCAAGTCCTTTCTTATCTATCCTGTCACGTGCCTCATAGCCATTTATATAGGCAAGACAGGCATTAGG
CTGCCCATCTTGTAGATGAGTAAACTGAGGCCCAGAGAGGGGAAATATATTGCAAGTTGGTAGCAGAA
TTGAGGTCTCTGCACAACTCAAATATGCCACAGTGCCTCCTTGTGGAGAGGAGGACAAAAGCAGAGCT
GAAATCATTATCTTGAAGAGGTGTCAGAAGTGGGATTGCGACAGGACTGATGTGATATTTTTAGATAT
GGCCAAGAGGACACAGTCTGAGTTTTTAGCTGAGAAATGTCCTCTATAAGGCAGAAGGCAGAGATTCT
AGAGGACCTTTGAGGGAGAATGTATTTGAGAACAACTCTTCCAGCTTCTTACATATGTACAGGTATCT
CTCAGGGGCTGACCTAGGAAGGGTCCTTTCCTGTGGCCATTGATCGATCCAGTCCCACATCTGGAAAG
CTTACAAGAATTGGGTTCAAAGCGGGGATTACACTTGATAATTACAGAAGGACCACCTACTTCTTAGA
GGAAAGACGCTGGGAGGTTGCTTAGGATGTGGGCCAAGAGGGTCAGAGAGGACCACCTACTTTTTAGA
GGAAAGACGCTGGGAGGTTGCTTAGGATGTGGGCCAAGAGGGTCAGAGATTTTGCTTCACCTGAACTC
ACTGGGGCTTCTCCAGGGATATTAACCTGGACTTTAAGAGTCAGAGTGAGTCCCTGGGACTAGTTCAG
CCCATCCAGGATTCAGACGGGAAGAAGGTGGGGCTGATTTTTCACCTGGAGAAAGAGAGGCATGTCCC
ACACAGACCTAACTCGGCATTGTCCCCTCCCAAACTCCCACCCCTCCACATAGCTTAAAAGTGTTGGG
GGCTTCTCCAGTTTAGATGGGGGAACAAAGAGAACCAACAGCTGGAAAAAACTAGAGATGAGGCCGTT
GGCCTAGTCATCATCCAGGCCGATTTCTCAGAACCACCACTTTCTCTTCGGCTACTTTGCCCATCCCA
TAAAAGAACCCCAAATCCTTCCTGTTCATTCCTCAGCAGTTCCCACGTTTCCTTCCAGAAACTCAGAA
GGCACCAGGAACTGAATTGCAAAGTTCGTTAGAGCACAGACTCTGAATTAAAGAGCTGGGTTAAACTC
CAGGCTATTCCCTTAGTAGCTGTGTGACCTTACCTGTCTGAAGCTTGGTTTTCTCCCAGTAAGATGGG
GTAGTACTGCCTAAAGAGGTATATGGCATGTATAAAGTGCTCCATAAATGGAGCTTATTGGGAGAGTA
TAAGTCACAGGCCATGCCCCGCAAGGGGATGCACGAAGACCCACCGCGAGCCAGGAAGGGAGCACCGG
GCTCTCTGCTCTGGGACCGGCAGTGAGCCGGACATCTGGGTCCTCCCAAGCCGGGCGGGCTGCCCCAG
GGAGGAAGGGAGGGGGGCGAGCCTGAGCGGGCACCTCGGCCCGCAGGAGGTCTGCAGCGAGCTGTGGT
GTCTGAGCAAGAGCAACCGGTGCATCACCAACAGCATCCCGGCCGCCGAGGGCACGCTGTGCCAGACG
CACACCATCGACAAGGGGTGGTGCTACAAACGGGTCTGTGTCCCCTTTGGGTCGCGCCCAGAGGGTGT
GGACGGAGCCTGGGGGCCGTGGACTCCATGGGGCGACTGCAGCCGGACCTGTGGCGGCGGCGTGTCCT
CTTCTAGCCGTCACTGCGACAGCCCCAGGCCAACCATCGGGGGCAAGTACTGTCTGGGTGAGAGAAGG
CGGCACCGCTCCTGCAACACGGATGACTGTCCCCCTGGCTCCCAGGACTTCAGAGAAGTGCAGTGTTC
TGAATTTGACAGCATCCCTTTCCGTGGGAAATTCTACAAGTGGAAAACGTACCGGGGAGGGGGCGTGA
AGGCCTGCTCGCTCACGTGCCTAGCGGAAGGCTTCAACTTCTACACGGAGAGGGCGGCAGCCGTGGTG
GACGGGACACCCTGCCGTCCAGACACGGTGGACATTTGCGTCAGTGGCGAATGCAAGCACGTGGGCTG
CGACCGAGTCCTGGGCTCCGACCTGCGGGAGGACAAGTGCCGAGTGTGTGGCGGTGACGGCAGTGCCT
GCGAGACCATCGAGGGCGTCTTCAGCCCAGCCTCACCTGGGGCCGGGTACGAGGATGTCGTCTGGATT
CCCAAAGGCTCCGTCCACATCTTCATCCAGGATCTGAACCTCTCTCTCAGTCACTTGGCCCTGAAGGG
AGACCAGGAGTCCCTGCTGCTGGAGGGGCTGCCCGGGACCCCCCAGCCCCACCGTCTGCCTCTAGCTG
GGACCACCTTTCAACTGCGACAGGGGCCAGACCAGGTCCAGAGCCTCGAAGCCCTGGGACCGATTAAT
GCATCTCTCATCGTCATGGTGCTGGCCCGGACCGAGCTGCCTGCCCTCCGCTACCGCTTCAATGCCCC
CATCGCCCGTGACTCGCTGCCCCCCTACTCCTGGCACTATGCGCCCTGGACCAAGTGCTCGGCCCAGT
GTGCAGGCGGTAGCCAGGTGCAGGCGGTGGAGTGCCGCAACCAGCTGGACAGCTCCGCGGTCGCCCCC
CACTACTGCAGTGCCCACAGCAAGCTGCCCAAAAGGCAGCGCGCCTGCAACACGGAGCCTTGCCCTCC
AGACTGGGTTGTAGGGAACTGGTCGCTCTGCAGCCGCAGCTGCGATGCAGGCGTGCGCAGCCGCTCGG
TCGTGTGCCAGCGCCGCGTCTCTGCCGCGGAGGAGAAGGCGCTGGACGACAGCGCATGCCCGCAGCCG
CGCCCACCTGTACTGGAGGCCTGCCACGGCCCCACTTGCCCTCCGGAGTGGGCGGCCCTCGACTGGTC
TGAGTGCACCCCCAGCTGCGGGCCGGGCCTCCGCCACCGCGTGGTCCTTTGCAAGAGCGCAGACCACC
GCGCCACGCTGCCCCCGGCGCACTGCTCACCCGCCGCCAAGCCACCGGCCACCATGCGCTGCAACTTG
CGCCGCTGCCCCCCGGCCCGCTGGGTGGCTGGCGAGTGGGGTGAGTGCTCTGCACAGTGCGGCGTCGG
GCAGCGGCAGCGCTCGGTGCGCTGCACCAGCCACACGGGCCAGGCGTCGCACGAGTGCACGGAGGCCC
TGC

| | | | |
|---|---|---|---|
| NOV17d, CG51213-02 | SEQ ID NO: 202 | 1634 aa | MW at 68853.1kD |
| Protein Sequence | | | |

YCLKRYMACIKCSINGAYWESISHRPCPARGCTKTHREPGREHRALCSGTGSEPDIWVLPSRAGCPRE
EGRGASLSGHLGPQEVCSELWCLSKSNRCIThSIPAAEGTLCQTHTIDKGWCYKRVCVPFGSRPEGVD
GAWGPWTPWGDCSRTCGGGVSSSSRHCDSPRPTIGGKYCLGERRRHRSCNTDDCPPGSQDFREVQCSE
FDSIPFRGKFYKWKTYRGGGVKACSLTCLAEGFNFYTERAAAVVDGTPCRPDTVDICVSGECKHVGCD
RVLGSDLREDKCRVCGGDGSACETIEGVFSPASPGAGYEDVVWIPKGSVHIFIQDLNLSLSHLALKGD
QESLLLEGLPGTPQPHRLPLAGTTFQLRQGPDQVQSLEALGPINASLIVMVLARTELPALRYRFNAPI
ARDSLPPYSWHYAPWTKCSAQCAGGSQVQAVECRNQLDSSAVAPHYCSAHSKLPKRQRACNTEPCPPD
WVVGNWSLCSRSCDAGVRSRSVVCQRRVSAAEEKALDDSACPQPRPPVLEACHGPTCPPEWAALDWSE
CTPSCGPGLRHRVVLCKSADHRATLPPAHCSPAAKPPATMRCNLRRCPPARWVAGEWGECSAQCGVGQ
RQRSVRCTSHTGQASHECTEAL

| | | |
|---|---|---|
| NOV17e, CG51213-03 | SEQ ID NO: 203 | 3700 bp |
| DNA Sequence | ORF Start: at 1798 | ORF Stop: at 3700 |

CTGACATTCCACCCTTGACACCCCCCAACATCCTAACTTAGCTGGTAACTGCAGCACCCTCTAAGGAA
TTCCTAAAGAATTCTGAAGCTACTCCTCAACATCTGCTGTGACCCAGGTATCCTAACAATGATCATGG
TGTCTGACATTTACTGAGCTCTCACTATGGGCTAAGCATGTGCTGTGTGTCACCATCTAAACTCCTGA
CAATCCTGCTAGCCCCCACGTTACAGAGGAAGGGACTGAGCCATAGCATAGGGAGGATGACTTGTCCA
AGGCCACAGTTTGAGACCATGACAGAGCTGGGATTTAAATCCAGGTCTCTCATGACTCTCTAAATTTT
ACAAAGGGGCAGGGGAGGGGAGGAGCTGTCAAAATATCAAGCTTGGGCTGGCACTGGCTATATGTTGA
ATTGAGCCTTCCTTTTAGTTTTTGAAGGAACATCTTTCAGGCCATCTTGGCAAAGGGGGATTTATTTA
CTAAATGTGAACTGGTTAATATATGTAAAGGGTTCAGCCTGGTGCCTGGTCCAGAGATAGTGGTGGTC
ATTGTTACCCCATAATGGCATTGGTGCAAGTCCTTTCTTATCTATCCTGTCACGTGCCTCATAGCCAT
TTATATAGGCAAGACAGGCATTAGGCTGCCCATCTTGTAGATGAGTAAACTGAGGCCCAGAGAGGGGA
AATATATTGCAAGTTGGTAGCAGAATTGAGGTCTCTGCACAACTCAAATATGCCACAGTGCCTCCTTG
TGGAGAGGAGGACAAAAGCAGAGCTGAAATCATTATCTTGAAGAGGTGTCAGAAGTGGGATTGCGACA
GGACTGATGTGATATTTTTAGATATGGCCAAGAGGACACAGTCTGAGTTTTTAGCTGAGAAATGTCCT
CTATAAGGCAGAAGGCAGAGATTCTAGAGGACCTTTGAGGGAGAATGTATTTGAGAACAACTCTTCCA
GCTTCTTACATATGTACAGGTATCTCTCAGGGGCTGACCTAGGAAGGGTCCTTTCCTGTGGCCATTGA
TCGATCCAGTCCCACATCTGGAAAGCTTACAAGAATTGGGTTCAAAGCGGGGATTACACTTGATAATT
ACAGAAGGACCACCTACTTCTTAGAGGAAAGACGCTGGGAGGTTGCTTAGGATGTGGGCCAAGAGGGT
CAGAGAGGACCACCTACTTTTTAGAGGAAAGACGCTGGGAGGTTGCTTAGGATGTGGGCCAAGAGGGT
CAGAGATTTTGCTTCACCTGAACTCACTGGGGCTTCTCCAGGGATATTAACCTGGACTTTAAGAGTCA
GAGTGAGTCCCTGGGACTAGTTCAGCCCATCCAGGATTCAGACGGGAAGAAGGTGGGGCTGATTTTTC
ACCTGGAGAAAGAGAGGCATGTCCCACACAGACCTAACTCGGCATTGTCCCCTCCCAAACTCCCACCC
CTCCACATAGCTTAAAAGTGTTGGGGGCTTCTCCAGTTTAGATGGGGGAACAAAGAGAACCAACAGCT
GGAAAAAACTAGAGATGAGGCCGTTGGCCTAGTCATCATCCAGGCCGATTTCTCAGAACCACCACTTT
CTCTTCGGCTACTTTGCCCATCCCATAAAAGAACCCCAAATCCTTCCTGTTCATTCCTCAGCAGTTCC
CACGTTTCCTTCCAGAAACTCAGAAGGCACCAGGAACTGAATTGCAAAGTTCGTTAGAGCACAGACTC
TGAATTAAAGAGCTGGGTTAAACTCCAGGCTATTCCCTTAGTAGCTGTGTGACCTTACCTGTCTGAAG
CTTGGTTTTCTCCCAGTAAGATGGGGTAGTACTGCCTAAAGAGGTATATGGCATGTATAAAGTGCTCC
ATAAATGGAGCTTATTGGGAGAGTATAAGTCACAGGCCATGCCCCGCAAGGGGATGCACGAAGACCCA
CCGCGAGCCAGGAAGGGAGCACGGGGCTCTCTGCTCTGGGACCGGCAGTGAGCCGGACATCTGGGTCC
TCCCAAGCCGGGCGGGCTGCCCCAGGGAGGAAGGGAGGGGGGCGAGCCTGAGCGGGCACCTCGGCCCG
CAGGAGGTCTGCAGCGAGCTGTGGTGTCTGAGCAAGAGCAACCGGTGCATCACCAACAGCATCCCGGC
CGCCGAGGGCACGCTGTGCCAGACGCACACCATCGACAAGGGGTGGTGCTACAAACGGGTCTGTGTCC
CCTTTGGGTCGCGCCCAGAGGGTGTGGACGGAGCCTGGGGGCCGTGGACTCCATGGGGCGACTGCAGC
CGGACCTGTGGCGGCGGCGTGTCCTCTTCTAGCCGTCACTGCGACAGCCCCAGGCCAACCATCGGGGG
CAAGTACTGTCTGGGTGAGAGAAGGCGGCACCGCTCCTGCAACACGGATGACTGTCCCCCTGGCTCCC
AGGACTTCAGAGAAGTGCAGTGTTCTGAATTTGACAGCATCCCTTTCCGTGGGAAATTCTACAAGTGG
AAAACGTACCGGGGAGGGGGCGTGAAGGCCTGCTCGCTCACGTGCCTAGCGGAAGGCTTCAACTTCTA
CACGGAGAGGGCGGCAGCCGTGGTGGACGGGACACCCTGCCGTCCAGACACGGTGGACATTTGCGTCA
GTGGCGAATGCAAGCACGTGGGCTGCGACCGAGTCCTGGGCTCCGACCTGCGGGAGGACAAGTGCCGA
GTGTGTGGCGGTGACGGCAGTGCCTGCGAGACCATCGAGGGCGTCTTCAGCCCAGCCTCACCTGGGGC
CGGGTACGAGGATGTCGTCTGGATTCCCAAAGGCTCCGTCCACATCTTCATCCAGGATCTGAACCTCT
CTCTCAGTCACTTGGCCCTGAAGGGAGACCAGGAGTCCCTGCTGCTGGAGGGGCTGCCCGGGACCCCC
CAGCCCCACCGTCTGCCTCTAGCTGGGACCACCTTTCAACTGCGACAGGGGCCAGACCAGGTCCAGAG
CCTCGAAGCCCTGGGACCGATTAATGCATCTCTCATCGTCATGGTGCTGGCCCGGACCGAGCTGCCTG
CCCTCCGCTACCGCTTCAATGCCCCCATCGCCCGTGACTCGCTGCCCCCCTACTCCTGGCACTATGCG
CCCTGGACCAAGTGCTCGGCCCAGTGTGCAGGCGGTAGCCAGGTGCAGGCGGTGGAGTGCCGCAACCA
GCTGGACAGCTCCGCGGTCGCCCCCCACTACTGCAGTGCCCACAGCAAGCTGCCCAAAAGGCAGCGCG
CCTGCAACACGGAGCCTTGCCCTCCAGACTGGGTTGTAGGGAACTGGTCGCTCTGCAGCCGCAGCTGC
GATGCAGGCGTGCGCAGCCGCTCGGTCGTGTGCCAGCGCCGCGTCTCTGCCGCGGAGGAGAAGGCGCT
GGACGACAGCGCATGCCCGCAGCCGCGCCCACCTGTACTGGAGGCCTGCCACGGCCCCACTTGCCCTC
CGGAGTGGGCGGCCCTCGACTGGTCTGAGTGCACCCCCAGCTGCGGGCCGGGCCTCCGCCACCGCGTG
GTCCTTTGCAAGAGCGCAGACCACCGCGCCACGCTGCCCCCGGCGCACTGCTCACCCGCCGCCAAGCC
ACCGGCCACCATGCGCTGCAACTTGCGCCGCTGCCCCCCGGCCCGCTGGGTGGCTGGCGAGTGGGGTG
AGTGCTCTGCACAGTGCGGCGTCGGGCAGCGGCAGCGCTCGGTGCGCTGCACCAGCCACACGGGCCAG
GCGTCGCACGAGTGCACGGAGGCCCTGC

| | | | |
|---|---|---|---|
| NOV17e, CG51213-03 | SEQ ID NO: 204 | 634 aa | MW at 68754.0kD |
| Protein Sequence | | | |

YCLKRYMACIKCSINGAYWESISHRPCPARGCTKTHREPGREHGALCSGTGSEPDIWVLPSRAGCPRE
EGRGASLSGHLGPQEVCSELWCLSKSNRCITNSIPAAEGTLCQTHTIDKGWCYKRVCVPFGSRPEGVD
GAWGPWTPWGDCSRTCGGGVSSSSRHCDSPRPTIGGKYCLGERRRBRSCNTDDCPPGSQDFREVQCSE
FDSIPFRGKFYKWKTYRGGGVKACSLTCLAEGFNFYTERAAAVVDGTPCRPDTVDICVSGECKHVGCD
RVLGSDLREDKCRVCGGDGSACETIEGVFSPASPGAGYEDVVWIPKGSVHIFIQDLNLSLSHLALKGD
QESLLLEGLPGTPQPHRLPLAGTTFQLRQGPDQVQSLEALGPINASLIVMVLARTELPALRYRFNAPI
ARDSLPPYSWHYAPWTKCSAQCAGGSQVQAVECRNQLDSSAVAPHYCSAHSKLPKRQRACNTEPCPPD
WVVGNWSLCSRSCDAGVRSRSVVCQRRVSAAEEKALDDSACPQPRPPVLEACHGPTCPPEWAALDWSE
CTPSCGPGLRHRVVLCKSADHRATLPPAHCSPAAKPPATMRCNLRRCPPARNVAGEWGECSAQCGVGQ
RQRSVRCTSHTGQASHECTEAL

| | | |
|---|---|---|
| NOV17f, CG51213-05 | SEQ ID NO: 205 | 3400 bp |
| DNA Sequence | ORF Start: at 1 | ORF Stop: TAG at 3232 |

CGGTCTCAAGATGAGTTCCTGTCCAGTCTGGAGAGCTATGAGATCGCCTTCCCCACCCGCGTGGACCA
CAACGGGGCACTGCTGGCCTTCTCGCCACCTCCTCCCCGGAGGCAGCGCCGCGGCACGGGGGCCACAG
CCGAGTCCCGCCTCTTCTACAAAGTAGCCTCGCCCAGCACCCACTTCCTGCTGAACCTGACCCGCAGC
TCCCGTCTACTGGCAGGGCACGTCTCCGTGGAGTACTGGACACGGGAGGGCCTGGCCTGGCAGAGGGC
GGCCCGGCCCCACTGCCTCTACGCTGGTCACCTGCAGGGCCAGGCCAGCAGCTCCCATGTGGCCATCA
GCACCTGTGGAGGCCTGCACGGCCTGATCGTGGCAGACGAGGAAGAGTACCTGATTGAGCCCCTGCAC
GGTGGGCCCAAGGGTTCTCGGAGCCCGGAGGAAAGTGGACCACATGTGGTGTACAAGCGTTCCTCTCT
GCGTCACCCCCACCTGGACACAGCCTGTGGAGTGAGAGATGAGAAACCGTGGAAAGGGCGGCCATGGT
GGCTGCGGACCTTGAAGCCACCGCCTGCCAGACCCCTGGGGAATGAAACAGAGCGTGGCCAGCCAGGC
CTGAAGCGATCGGTCAGCCGAGAGCGCTACGTGGAGACCCTGGTGGTGGCTGACAAGATGATGGTGGC
CTATCACGGGCGCCGGGATGTGGAGCAGTATGTCCTGGCCATCATGAACATTGTTGCCAAACTTTTCC
AGGACTCGAGTCTGGGAAGCACCGTTAACATCCTCGTAACTCGCCTCATCCTGCTCACGGAGGACCAG
CCCACTCTGGAGATCACCCACCATGCCGGGAAGTCCCTAGACAGCTTCTGTAAGTGGCAGAAATCCAT
CGTGAACCACAGCGGCCATGGCAATGCCATTCCAGAGAACGGTGTGGCTAACCATGACACAGCAGTGC
TCATCACACGCTATGACATCTGCATCTACAAGAACAAACCCTGCGGCACACTAGGCCTGGCCCCGGTG
GGCGGAATGTGTGAGCGCGAGAGAAGCTGCAGCGTCAATGAGGACATTGGCCTGCCACAAGCGTTCAC
CATTGCCCACGAGATCGGGCACACATTCGGCATGAACCATGACGGCGTGGGAAACAGCTGTGGGGCCC
GTGGTCAGGACCCAGCCAAGCTCATGGCTGCCCACATTACCATGAAGACCAACCCATTCGTGTGGTCA
TCCTGCAACCGTGACTACATCACCAGCTTTCTAGACTCGGGCCTGGGGCTCTGCCTGAACAACCGGCC
CCCCAGACAGGACTTTGTGTACCCGACAGTGGCACCGGGCCAAGCCTACGATGCAGATGAGCAATGCC
GCTTTCAGCATGGAGTCAAATCGCGTCAGTGTAAATACGGGGAGGTCTGCAGCGAGCTGTGGTGTCTG
AGCAAGAGCAACCGGTGCATCACCAACAGCATCCCGGCCGCCGAGGGCACGCTGTGCCAGACGCACAC
CATCGACAAGGGGTGGTGCTACAAACGGGTCTGTGTCCCCTTTGGGTCGCGCCCAGAGGGTGTGGACG
GAGCCTGGGGGCCGTGGACTCCATGGGGCGACTGCAGCCGGACCTGTGGCGGCGGCGTGTCCTCTTCT
AGTCGTCACTGCGACAGCCCCAGGCCAACCATCGGGGGCAAGTACTGTCTGGGTGAGAGAAGGCGGCA
CCGCTCCTGCAACACGGATGACTGTCCCCCTGGCTCCCAGGACTTCAGAGAAGTGCAGTGTTCTGAAT
TTGACAGCATCCCTTTCCGTGGGAAATTCTACAAGTGGAAAACGTACCGGGGAGGGGGCGTGAAGGCC
TGCTCGCTCACGAGCCTAGCGGAAGGCTTCAACTTCTACACGGAGAGGGCGGCAGCCGTGGTGGACGG
GACACCCTGCCGTCCAGACACGGTGGACATTTGCGTCAGTGGCGAATGCAAGCACGTGGGCTGCGACC
GAGTCCTGGGCTCCGACCTGCGGGAGGACAAGTGCCGAGTGTGTGGCGGTGACGGCAGTGCCTGCGAG
ACCATCGAGGGCGTCTTCAGCCCAGCCTCACCTGGGGCCGGGTACGAGGATGTCGTCTGGATTCCCAA
AGGCTCCGTCCACATCTTCATCCAGGATCTGAACCTCTCTCTCAGTCACTTGGCCCTGAAGGGAGACC
AGGAGTCCCTGCTGCTGGAGGGGCTGCCTGGGACCCCCCAGCCCCACCGTCTGCCTCTAGCTGGGACC
ACCTTTCAACTGCGACAGGGGCCAGACCAGGTCCAGAGCCTCGAAGCCCTGGGACCGATTAATGCATC
TCTCATCGTCATGGTGCTGGCCCGGACCGAGCTGCCTGCCCTCCGCTACCGCTTCAATGCCCCCATCG
CCCGTGACTCGCTGCCCCCCTACTCCTGGCACTATGCGCCCTGGACCAAGTGCTCGGCCCAGTGTGCA
GGCGGTAGCCAGGTGCAGGCGGTGGAGTGCCGCAACCAGCTGGACAGCTCCGCGGTCGCCCCCCACTA
CTGCAGTGCCCACAGCAAGCTGCCCAAAAGGCAGCGCGCCTGCAACACGGAGCCTTGCCCTCCAGACT
GGGTTGTAGGGAACTGGTCGCTCTGCAGCCGCAGCTGCGATGCAGGCGTGCGCAGTCGCTCGGTCGTG
TGCCAGCGCCGCGTCTCTGCCGCGGAGGAGAAGGCGCTGGACGACAGCGCATGCCCGCAGCCGCGCCC
ACCTGTACTGGAGGCCTGCCACGGCCCCACTTGCCCTCCGGAGTGGGCGGCCCTCGACTGGTCTGAGT
GCACCCCCAGCTGCGGGCCGGGCCTCCGCCACCGCGTGGTCCTTTGCAAGAGCGCAGACCACCGCGCC
ACGCTGCCCCCGGCGCACTGCTCACCCGCCGCCAAGCCACCGGCCACCATGCGCTGCAACTTGCGCCG
CTGCCCCCCGGCCCGCTGGGTGGCTGGCGAGTGGGGTGAGTGCTCTGCACAGTGCGGCGTCGGGCAGC
GGCAGCGCTCGGTGCGCTGCACCAGCCACACGGGCCAGGCGTCGCACGAGTGCACGGAGGCCCTGCGG
CCGCCCACCACGCAGCAGTGTGAGGCCAAGTGCGACAGCCCAACCCCCGGGGACGGCCCTGAAGAGTG
CAAGGATGTGAACAAGGTCGCCTACTGCCCCCTGGTGCTCAAATTTCAGTTCTGCAGCCGAGCCTACT
TCCGCCAGATGTGCTGCAAAACCTGCCAGGGCCACTAGGGGGCGCGCGGCACCCGGAGCCACAGCTGG
CGGGGTCTCCGCCGCCAGCCCTGCAGCGGGCCGGCCAAAGGGGGCCCCGGGGGGGCGGGAACTGGGAG
GGAAGGGTGAGACGGAGCCGGAAGTTATTTATTGGGAACCCCTGCAGGGCCCTGGCTGGGGGGATGGA

| | | | |
|---|---|---|---|
| NOV17f, CG51213-05 | SEQ ID NO: 206 | 1077 aa | MW at 118071.4kD |
| Protein Sequence | | | |

RSQDEFLSSLESYEIAFPTRVDHNGALLAFSPPPPRRQRRGTGATAESRLFYKVASPSTHFLLNLTRS
SRLLAGHVSVEYWTREGLAWQRAARPHCLYAGHLQGQASSSHVAISTCGGLHGLIVADEEEYLIEPLH
GGPKGSRSPEESGPHVVYKRSSLRHPHLDTACGVRDEKPWKGRPWWLRTLKPPPARPLGNETERGQPG
LKRSVSRERYVETLVVADKMMVAYHGRRDVEQYVLAIMNIVAKLFQDSSLGSTVNILVTRLILLTEDQ
PTLEITHHAGKSLDSFCKWQKSIVNHSGHGNAIPENGVANHDTAVLITRYDICIYKNKPCGTLGLAPV
GGMCERERSCSVNEDIGLPQAFTIAHEIGHTFGMNHDGVGMSCGARGQDPAKLMAAHITNKTNPFVWS
SCNRDYITSFLDSGLGLCLNNRPPRQDFVYPTVAPGQAYDADEQCRFQHGVKSRQCKYGEVCSELWCL
SKSNRCITNSIPAAEGTLCQTHTIDKGWCYKRVCVPFGSRPEGVDGAWGPWTPWGDCSRTCGGGVSSS
SRHCDSPRPTIGGKYCLGERRRHRSCNTDDCPPGSQDFREVQCSEFDSIPFRGKFYKWKTYRGGGVKA
CSLTSLAEGFNFYTERAAAVVDGTPCRPDTVDICVSGECKHVGCDRVLGSDLREDKCRVCGGDGSACE
TIEGVFSPASPGAGYEDVVWIPKGSVHIFIQDLNLSLSHLALKGDQESLLLEGLPGTPQPHRLPLAGT
TFQLRQGPDQVQSLEALGPINASLIVMVLARTELPALRYRFNAPIARDSLPPYSWHYAPWTKCSAQCA
GGSQVQAVECRNQLDSSAVAPHYCSAHSKLPKRQRACNTEPCPPDWVVGNWSLCSRSCDAGVRSRSVV
CQRRVSAAEEKALDDSACPQPRPPVLEACHGPTCPPEWAALDWSECTPSCGPGLRHRVVLCKSADHPA
TLPPAHCSPAAKPPATMRCNLRRCPPARWVAGEWGECSAQCGVGQRQRSVRCTSHTGQASHECTEALR
PPTTQQCEAKCDSPTPGDGPEECKDVNKVAYCPLVLKFQFCSRAYFRQMCCKTCQGH

| | | |
|---|---|---|
| NOV17g, CG51213-06 | SEQ ID NO: 207 | 978 bp |
| DNA Sequence | ORF Start: at I | ORF Stop: end of sequence |

TCCATAAATGGAGCTTATTGGGAGAGTATAAGTCACAGGCCATGCCCCGCAAGGGGATGCACGAAGAC
CCACCGCGAGCCAGGAAGGGAGCACCGGGCTCTCTGCTCTGGGACCGGCAGTGAGCCGGACATCTGGG
TCCTCCCAAGCCGGGCGGGCTGCCCCAGGGAGGAAGGGAGGGGGGCGAGCCTGAGCGGGCACCTCGGC
CCGCAGGAGGTCTGCAGCGAGCTGTGGTGTCTGAGCAAGAGCAACCGGTGCATCACCAACAGCATCCC
GGCCGCCGAGGGCACGCTGTGCCAGACGCACACCATCGACAAGGGGTGGTGCTACAAACGGGTCTGTG
TCCCCTTTGGGTCGCGCCCAGAGGGTGTGGACGGAGCCTGGGGGCCGTGGACTCCATGGGGCGACTGC
AGCCGGACCTGTGGCGGCGGCGTGTCCTCTTCTAGCCGTCACTGCGACAGCCCCAGGCCAACCATCGG
GGGCAAGTACTGTCTGGGTGAGAGAAGGCGGCACCGCTCCTGCAACACGGATGACTGTCCCCCTGGCT
CCCAGGACTTCAGAGAAGTGCAGTGTTCTGAATTTGACAGCATCCCTTTCCGTGGGAAATTCTACAAG
TGGAAAACGTACCGGGGAGGGGGCGTGAAGGCCTGCTCGCTCACGTGCCTAGCGGAAGGCTTCAACTT
CTACACGGAGAGGGCGGCAGCCGTGGTGGACGGGACACCCTGCCGTCCAGACACGGTGGACATTTGCG
TCAGTGGCGAATGCAAGCACGTGGGCTGCGACCGAGTCCTGGGCTCCGACCTGCGGGAGGACAAGTGC
CGAGTGTGTGGCGGTGACGGCAGTGCCTGCGAGACCATCGAGGGCGTCTTCAGCCCAGCCTCACCTGG
GGCCGGGTACGAGGATGTCGTCTGGATTCCCAAAGGCTCCGTCCACATCTTCATCCAGGATCTGAACC
TCTCTCTCAGTCACTTGGCCCTGAAG

| | | | |
|---|---|---|---|
| NOV17g, CG51213-06 | SEQ ID NO: 208 | 326 aa | MW at 35330.2kD |
| Protein Sequence | | | |

SINGAYWESISHRPCPARGCTKTHREPGREHRALCSGTGSEPDIWVLPSRAGCPREEGRGASLSGHLG
PQEVCSELWCLSKSNRCITNSIPAAEGTLCQTHTIDKGWCYKRVCVPFGSRPEGVDGAWGPWTPWGDC
SRTCGGGVSSSSRHCDSPRPTIGGKYCLGERRRHRSCNTDDCPPGSQDFREVQCSEFDSIPFRGKFYK
WKTYRGGGVKACSLTCLAEGFNFYTERAAAVVDGTPCRPDTVDICVSGECKHVGCDRVLGSDLREDKC
RVCGGDGSACETIEGVFSPASPGAGYEDVVWIPKGSVHIFIQDLNLSLSHLALK

| | | |
|---|---|---|
| NOV17h, CG51213-07 | SEQ ID NO: 209 | 1866 bp |
| DNA Sequence | ORF Start: at 1 | ORF Stop: end of sequence |

TCCATAAATGGAGCTTATTGGGAGAGTATAAGTCACAGGCCATGCCCCGCAAGGGGATGCACGAAGAC
CCACCGCGAGCCAGGAAGGGAGCACCGGGCTCTCTGCTCTGGGACCGGCAGTGAGCCGGACATCTGGG
TCCTCCCAAGCCGGGCGGGCTGCCCCAGGGAGGAAGGGAGGGGGGCGAGCCTGAGCGGGCACCTCGGC
CCGCAGGAGGTCTGCAGCGAGCTGTGGTGTCTGAGCAAGAGCAACCGGTGCATCACCAACAGCATCCC
GGCCGCCGAGGGCACGCTGTGCCAGACGCACACCATCGACAAGGGGTGGTGCTACAAACGGGTCTGTG
TCCCCTTTGGGTCGCGCCCAGAGGGTGTGGACGGAGCCTGGGGGCCGTGGACTCCATGGGGCGACTGC
AGCCGGACCTGTGGCGGCGGCGTGTCCTCTTCTAGCCGTCACTGCGACAGCCCCAGGCCAACCATCGG
GGGCAAGTACTGTCTGGGTGAGAGAAGGCGGCACCGCTCCTGCAACACGGATGACTGTCCCCCTGGCT
CCCAGGACTTCAGAGAAGTGCAGTGTTCTGAATTTGACAGCATCCCTTTCCGTGGGAAATTCTACAAG
TGGAAAACGTACCGGGGAGGGGGCGTGAAGGCCTGCTCGCTCACGTGCCTAGCGGAAGGCTTCAACTT
CTACACGGAGAGGGCGGCAGCCGTGGTGGACGGGACACCCTGCCGTCCAGACACGGTGGACATTTGCG
TCAGTGGCGAATGCAAGCACGTGGGCTGCGACCGAGTCCTGGGCTCCGACCTGCGGGAGGACAAGTGC
CGAGTGTGTGGCGGTGACGGCAGTGCCTGCGAGACCATCGAGGGCGTCTTCAGCCCAGCCTCACCTGG
GGCCGGGTACGAGGATGTCGTCTGGATTCCCAAAGGCTCCGTCCACATCTTCATCCAGGATCTGAACC
TCTCTCTCAGTCACTTGGCCCTGAAGGGAGACCAGGAGTCCCTGCTGCTGGAGGGGCTGCCCGGGACC
CCCCAGCCCCACCGTCTGCCTCTAGCTGGGACCACCTTTCAACTGCGACAGGGGCCAGACCAGGTCCA
GAGCCTCGAAGCCCTGGGACCGATTAATGCATCTCTCATCGTCATGGTGCTGGCCCGGACCGAGCTGC
CTGCCCTCCGCTACCGCTTCAATGCCCCCATCGCCCGTGACTCGCTGCCCCCCTACTCCTGGCACTAT
GCGCCCTGGACCAAGTGCTCGGCCCAGTGTGCAGGCGGTAGCCAGGTGCAGGCGGTGGAGTGCCGCAA
CCAGCTGGACAGCTCCGCGGTCGCCCCCCACTACTGCAGTGCCCACAGCAAGCTGCCCAAAAGGCAGC
GCGCCTGCAACACGGAGCCTTGCCCTCCAGACTGGGTTGTAGGGAACTGGTCGCTCTGCAGCCGCAGC
TGCGATGCAGGCGTGCGCAGCCGCTCGGTCGTGTGCCAGCGCCGCGTCTCTGCCGCGGAGGAGAAGGC
GCTGGACGACAGCGCATGCCCGCAGCCGCGCCCACCTGTACTGGAGGCCTGCCACGGCCCCACTTGCC
CTCCGGAGTGGGCGGCCCTCGACTGGTCTGAGTGCACCCCCAGCTGCGGGCCGGGCCTCCGCCACCGC
GTGGTCCTTTGCAAGAGCGCAGACCACCGCGCCACGCTGCCCCCGGCGCACTGCTCACCCGCCGCCAA
GCCACCGGCCACCATGCGCTGCAACTTGCGCCGCTGCCCCCCGGCCCGCTGGGTGGCTGGCGAGTGGG
GTGAGTGCTCTGCACAGTGCGGCGTCGGGCAGCGGCAGCGCTCGGTGCGCTGCACCAGCCACACGGGC
CAGGCGTCGCACGAGTGCACGGAGGCCCTG

| | | | |
|---|---|---|---|
| NOV17h, CG51213-07 | SEQ ID NO: 210 | 622 aa | MW at 67376.2kD |
| Protein Sequence | | | |

SINGAYWESISHRPCPARGCTKTHREPGREHRALCSGTGSEPDIWVLPSRAGCPREEGRGASLSGHLG
PQEVCSELWCLSKSNRCITNSIPAAEGTLCQTHTIDKGWCYKRVCVPFGSRPEGVDGAWGPWTPWGDC
SRTCGGGVSSSSRHCDSPRPTIGGKYCLGERRRHRSCNTDDCPPGSQDFREVQCSEFDSIPFRGKFYK
WKTYRGGGVKACSLTCLAEGFNFYTERAAAVVDGTPCRPDTVDICVSGECKHVGCDRVLGSDLREDKC
RVCGGDGSACETIEGVFSPASPGAGYEDVVWIPKGSVHIFIQDLNLSLSHLALKGDQESLLLEGLPGT
PQPHRLPLAGTTFQLRQGPDQVQSLEALGPINASLIVMVLARTELPALRYRFNAPIARDSLPPYSWHY
APWTKCSAQCAGGSQVQAVECRNQLDSSAVAPHYCSAHSKLPKRQRACNTEPCPPDWVVGNWSLCSRS
CDAGVRSRSVVCQRRVSAAEEKALDDSACPQPRPPVLEACHGPTCPPEWAALDWSECTPSCGPGLRHR
VVLCKSADHRATLPPAHCSPAAKPPATMRCNLRRCPPARWVAGEWGECSAQCGVGQRQRSVRCTSHTG
QASHECTEAL

A ClustalW comparison of the above protein sequences yields the following sequence alignment shown in Table 17B.

TABLE 17B
Comparison of the NOV17 protein sequences.

NOV17a ------------------------------------------------------------
NOV17b ------------------------------------------------------------
NOV17c ------------------------------------------------------------
NOV17d ------------------------------------------------------------
NOV17e ------------------------------------------------------------
NOV17f RSQDEFLSSLESYEIAFPTRVDHNGALLAFSPPPPRRQRRGTGATABSRLFYKVASPSTH
NOV17g ------------------------------------------------------------
NOV17h ------------------------------------------------------------
NOV17a ------------------------------------------------------------
NOV17b ------------------------------------------------------------
NOV17c ------------------------------------------------------------
NOV17d ------------------------------------------------------------
NOV17e ------------------------------------------------------------
NOV17f FLLNLTRSSRLLAGHVSVEYWTREGLAWQRAARPHCLYAGHLQGQASSSHVAISTCGGLH
NOV17g ------------------------------------------------------------
NOV17h ------------------------------------------------------------
NOV17a ------------------------------------------------------------
NOV17b ------------------------------------------------------------
NOV17c ------------------------------------------------------------
NOV17d ------------------------------------------------------------
NOV17e ------------------------------------------------------------
NOV17f GLIVADEEEYLIEPLHGGPKGSRSPEESGPHVVYKRSSLRHPHLDTACGVRDEKPWKGRP
NOV17g ------------------------------------------------------------
NOV17h ------------------------------------------------------------
NOV17a ------------------------------------------MMVAYHGRRDVEQYVLAI
NOV17b ------------------------------------------------------------
NOV17c ------------------------------------------------------------
NOV17d ------------------------------------------------------------
NOV17e ------------------------------------------------------------
NOV17f WWLRTLKPPPARPLGNETERGQPGLKRSVSRERYVETLVVADKMMVAYHGRRDVEQYVLA
NOV17g ------------------------------------------------------------
NOV17h ------------------------------------------------------------
NOV17a MNIQVAKLFQDSSLGSTVNILVTRLILLTEDQPTLEITHHAGKSLDSFCKWQKSIVNHSG
NOV17b ------------------------------------------------------------
NOV17c ------------------------------------------------------------
NOV17d ------------------------------------------------------------
NOV17e ------------------------------------------------------------
NOV17f IMNIVAKLFQDSSLGSTVNILVTRLILLTEDQPTLEITHHAGKSLDSFCKWQKSIVNHSG
NOV17g ------------------------------------------------------------
NOV17h ------------------------------------------------------------
NOV17a HGNAIPENGVANHDTAVLITRYDICIYKNKPCGTLGLAPVGGMCERERSCSVNEDIGLAT
NOV17b ------------------------------------------------------------
NOV17c ------------------------------------------------------------
NOV17d ------------------------------------------------------------
NOV17e ------------------------------------------------------------
NOV17f HGNAIPENGVANHDTAVLITRYDICIYKNKPCGTLGLAPVGGMCERERSCSVNEDIGLPQ
NOV17g ------------------------------------------------------------
NOV17h ------------------------------------------------------------
NOV17a AFTIAHEIGHTFGMNHDGVGMSCGARGQDPAKLMAAHITMKTNPFVWSSCSRDYITSFLD
NOV17b ------------------------------------------------------------
NOV17c ------------------------------------------------------------
NOV17d ------------------------------------------------------------
NOV17e ------------------------------------------------------------
NOV17f AFTIAHEIGHTFGMNHDGVGNSCGARGQDPAKLMAAHITMKTNPFVWSSCNRDYITSFLD
NOV17g ------------------------------------------------------------
NOV17h ------------------------------------------------------------
NOV17a SGLGLCLNNRPPRQDFVYPTVAPGQAYDADEQCRFQHGVKSRQCKYGEVCSELWCLSKSN
NOV17b ------------------------------------------------------------
NOV17c ------------------------------------------------------------
NOV17d ------------------------------YCLKRYMACIKCSINGAYWESISHRPCPAR
NOV17e ------------------------------YCLKRYMACIKCSINGAYWESISHRPCPAR
NOV17f SGLGLCLNNRPPRQDFVYPTVAPGQAYDADEQCRFQHGVKSRQCKYGEVCSELWCLSKSN
NOV17g ------------------------------------------SINGAYWESISHRPCPAR
NOV17h ------------------------------------------SINGAYWESISHRPCPAR
NOV17a RCITNSIPAAEGTLCQTHTIDKGWCYKRVCVPFGSRPEGVDGAWGPWTPWGDCSRTCGGG
NOV17b --------------------------------MGVQRD----AGSPHSLCPSCPSSWVWG
NOV17c ------------------------------------------------------------
NOV17d GCTKTHREPGREHRALCSGTGSEPDIWVLPSRAGCPREEGRGASLSGHLGPQEVCSELWC
NOV17e GCTKTHREPGREHGALCSGTGSEPDIWVLPSRAGCPREEGRGASLSGHLGPQEVCSELWC
NOV17f RCITNSIPAAEGTLCQTHTIDKGWCYKRVCVPFGSRPEGVDGAWGPWTPWGDCSRTCGGG
NOV17g GCTKTHREPGREHRALCSGTGSEPDIWVLPSRAGCPREEGRGASLSGHLGPQEVCSELWC
NOV17h GCTKTHREPGREHRALCSGTGSEPDIWVLPSRAGCPREEGRGASLSGHLGPQEVCSELWC
NOV17a VS-----------------------S--SS---------RHCD-----------------
NOV17b LS-----------------------L--LA---------SLPQ-----------------
NOV17c ------------------------------------------------------------
NOV17d LSKSNRCITNSIPAAEGTLCQTHTIDKGWCYKRVCVPFGSRPEGVDGAWGPWTPWGDCSR
NOV17e LSKSNRCITNSIPAAEGTLCQTHTIDKGWCYKRVCVPFGSRPEGVDGAWGPWTPWGDCSR
NOV17f VS-----------------------S--SS---------RHCD-----------------
NOV17g LSKSNRCITNSIPAAEGTLCQTHTIDKGWCYKRVCVPFGSRPEGVDGAWGPWTPWGDCSR
NOV17h LSKSNRCITNSIPAAEGTLCQTHTIDKGWCYKRVCVPFGSRPEGVDGAWGPWTPWGDCSR
NOV17a ---S-PR--P--------TIGGKYCLSERRRHRSCNTDDCPPGSQDFREVQCSEFDSIPF
NOV17b ---G-AS--K--------EFRGGLARG--------VTGPCPPG-----------------
NOV17c ------------------------------------------------------------
NOV17d TCGGGVSSSSRHCDSPRPTIGGKYCLGERRRHRSCNTDDCPPGSQDFREVQCSEFDSIPF
NOV17e TCGGGVSSSSRHCDSPRPTIGGKYCLGERRRHRSCNTDDCPPGSQDFREVQCSEFDSIPF
NOV17f ---S-PR--P--------TIGGKYCLGERRRHRSCNTDDCPPGSQDFREVQCSEFDSIPF
NOV17g TCGGGVSSSSRHCDSPRPTIGGKYCLGERRRHRSCNTDDCPPGSQDFREVQCSEFDSIPF
NOV17h TCGGGVSSSSRHCDSPRPTIGGKYCLGERRRHRSCNTDDCPPGSQDFREVQCSEFDSIPF
NOV17a RGKFYKWKTYRGGGVKACSLTSLAEGFNFYTERAAAVVDGTPCRPDTVDICVSGECKHVG
NOV17b -------------GVKACSLTCLAEGFNFYTERAAAVVDGTPCRPDTVDICVSGECKHVG
NOV17c ------------------------------------------------------------
NOV17d RGKFYKWKTYRGGGVKACSLTCLAEGFNFYTERAAAVVDGTPCRPDTVDICVSGECKHVG
NOV17e RGKFYKWKTYRGGGVKACSLTCLAEGFNFYTERAAAVVDGTPCRPDTVDICVSGECKHVG
NOV17f RGKFYKWKTYRGGGVKACSLTSLAEGFNFYTERAAAVVDGTPCRPDTVDICVSGECKHVG
NOV17g RGKFYKWKTYRGGGVKACSLTCLAEGFNFYTERAAAVVDGTPCRPDTVDICVSGECKHVG
NOV17h RGKFYKWKTYRGGGVKACSLTCLAEGFNFYTERAAAVVDGTPCRPDTVDICVSGECKHVG
NOV17a CDRVLGSDLREDKCRVCGGDGSACETIEGVFSPASPGAGYEDVVWIPKGSVHIFIQDLNL
NOV17b CDRVLGSDLREDKCRVCGGDGSACETIEGVFSPASPGAGYEDVVWIPKGSVHIFIQDLNL
NOV17c ------------------------------------------------------------
NOV17d CDRVLGSDLREDKCRVCGGDGSACETIEGVFSPASPGAGYEDVVWIPKGSVHIFIQDLNL
NOV17e CDRVLGSDLREDKCRVCGGDGSACETIEGVFSPASPGAGYEDVVWIPKGSVHIFIQDLNL
NOV17f CDRVLGSDLREDKCRVCGGDGSACETIEGVFSPASPGAGYEDVVWIPKGSVHIFIQDLNL
NOV17g CDRVLGSDLREDKCRVCGGDGSACETIEGVFSPASPGAGYEDVVWIPKGSVHIFIQDLNL
NOV17h CDRVLGSDLREDKCRVCGGDGSACETIEGVFSPASPGAGYEDVVWIPKGSVHIFIQDLNL
NOV17a SLSHLALKGDQESLLLEGLPGTPQPHRLPLAGTTFQLRQGPDQVQSLEALGPINASLIVM
NOV17b SLSHLALKGDQESLLLEGLPGTPQPHRLPLAGTTFQLRQGPDQVQSLEALGPINASLIVM
NOV17c ------------------------------------------------------------
NOV17d SLSHLALKGDQESLLLEGLPGTPQPHRLPLAGTTFQLRQGPDQVQSLEALGPINASLIVM
NOV17e SLSHLALKGDQESLLLEGLPGTPQPHRLPLAGTTFQLRQGPDQVQSLEALGPINASLIVM
NOV17f SLSHLALKGDQESLLLEGLPGTPQPHRLPLAGTTFQLRQGPDQVQSLEALGPINASLIVM
NOV17g SLSHLALK----------------------------------------------------
NOV17h SLSHLALKGDQESLLLEGLPGTPQPHRLPLAGTTFQLRQGPDQVQSLEALGPINASLIVM
NOV17a VLARTELPALRYRFNAPIARDSLPPYSWHYAPWTKCSAQCAGGSQVQAVECRNQLDSSAV
NOV17b VLARTELPALRYRFNAPIARDSLPPYSWHYAPWTKCSAQCAGGSQVQAVECRNQLDSSAV
NOV17c ------------------------TGSNHYAPWTKCSAQCAGGSQVQAVECRNQLDSSAV
NOV17d VLARTELPALRYRFNAPIARDSLPPYSWHYAPWTKCSAQCAGGSQVQAVECRNQLDSSAV
NOV17e VLARTELPALRYRFNAPIARDSLPPYSWHYAPWTKCSAQCAGGSQVQAVECRNQLDSSAV
NOV17f VLARTELPALRYRFNAPIARDSLPPYSWHYAPWTKCSAQCAGGSQVQAVECRNQLDSSAV
NOV17g ------------------------------------------------------------
NOV17h VLARTELPALRYRFNAPIARDSLPPYSWHYAPWTKCSAQCAGGSQVQAVECRNQLDSSAV
NOV17a APHYCSAHSKLPKRQRACNTEPCPPDWVVGNWSLCSRSCDAGVRSRSVVCQRRVSAAEEK
NOV17b APHYCSAHSKLPKRQRACNTEPCPPDWVVGNWSLCSRSCDAGVRSRSVVCQRRVSAAEEK
NOV17c APHYCSAHSKLPKRQRACNTEPCPPDNVVGNWSLCSRSCDAGVRSRSVVCQRRVSAAEEK
NOV17d APHYCSAHSKLPKRQRACNTEPCPPDWVVGNWSLCSRSCDAGVRSRSVVCQRRVSAAEEK
NOV17e APHYCSAHSKLPKRQRACNTEPCPPDWVVGNWSLCSRSCDAGVRSRSVVCQRRVSAAEEK
NOV17f APHYCSAHSKLPKRQRACNTEPCPPDWVVGNWSLCSRSCDAGVRSRSVVCQRRVSAAEEK
NOV17g ------------------------------------------------------------
NOV17h APHYCSAHSKLPKRQRACNTEPCPPDWVVGNWSLCSRSCDAGVRSRSVVCQRRVSAAEEK
NOV17a ALDDSACPQPRPPVLEACHGPTCPPEWAALDWSECTPSCGPGLRHRVVLCKSADHRATLP
NOV17b ALDDSACPQPRPPVLEACHGPTCPPEWAALDWSECTPSCGPGLRHRVVLCKSADHRATLP
NOV17d ALDDSACPQPRPPVLEACHGPTCPPEWAALDWSECTPSCGPGLRHRVVLCKSADHRATLP
NOV17d ALDDSACPQPRPPVLEACHGPTCPPEWAALDWSECTPSCGPGLRHRVVLCKSADHPATLP
NOV17e ALDDSACPQPRPPVLEACHGPTCPPEWAALDWSECTPSCGPGLRHRVVLCKSADHRATLP
NOV17f ALDDSACPQPRPPVLEACHGPTCPPEWAALDWSECTPSCGPGLRHRVVLCKSADHRATLP
NOV17g ------------------------------------------------------------
NOV17h ALDDSACPQPRPPVLEACHGPTCPPEWAALDWSECTPSCGPGLRHRVVLCKSADHRATLP
NOV17a PAHCSPAAKPPATMRCNLRRCPPARWVAGEWGECSAQCGVGQRQRSVRCTSHTGQASHEC
NOV17b PAHCSPAAKPPATMRCNLRRCPPARWVAGEWGECSAQCGVGQRQRSVRCTSHTGQASHEC
NOV17c PAHCSPAAKPPATMRCNLRRCPPARWVAGEWGECSAQCGVGQRQRSVRCTSHTGQASHEC
NOV17d PAHCSPAAKPPATMRCNLRRCPPARWVAGEWGECSAQCGVGQRQRSVRCTSHTGQASHEC
NOV17e PAHCSPAAKPPATMRCNLRRCPPARWVAGEWGECSAQCGVGQRQRSVRCTSHTGQASHEC
NOV17f PAHCSPAAKPPATMRCNLRRCPPARWVAGEWGECSAQCGVGQRQRSVRCTSHTGQASHEC
NOV17g ------------------------------------------------------------
NOV17h PAHCSPAAKPPATMRCNLRRCPPARWVAGEWGECSAQCGVGQRQRSVRCTSHTGQASHEC
NOV17a TEALRPPTTQQCEAKCDSPTPGDGPEECKDVNKVAYCPLVLKFQFCSRAYFRQMCCKTCQ
NOV17b TEALRPPTTQQCEAKCDSPTPGDGPEECKDVNKVAYCPLVLKFQFCSRAYFRQMCCKTCQ
NOV17c TEALRPPTTQQCEAKCLEG-----------------------------------------
NOV17d TEAL--------------------------------------------------------
NOV17e TEAL--------------------------------------------------------
NOV17f TEALRPPTTQQCEAKCDSPTPGDGPEECKDVNKVAYCPLVLKFQFCSRAYFRQMCCKTCQ
NOV17g ------------------------------------------------------------
NOV17h TEAL--------------------------------------------------------
NOV17a GH
NOV17b GH
NOV17c --
NOV17d --
NOV17e --
NOV17f GH
NOV17g --
NOV17h --
NOV17a (SEQ ID NO: 196)
NOV17b (SEQ ID NO: 198)
NOV17c (SEQ ID NO: 200)
NOV17d (SEQ ID NO: 202)
NOV17e (SEQ ID NO: 204)
NOV17f (SEQ ID NO: 206)
NOV17g (SEQ ID NO: 208)
NOV17h (SEQ ID NO: 210)

Further analysis of the NOV17a protein yielded the following properties shown in Table 17C.

TABLE 17C
Protein Sequence Properties NOV17a

SignalP analysis: No Known Signal Sequence Indicated
PSORT II analysis:

| PSG: | a new signal peptide prediction method |
|---|---|
| | N-region: length 10; pos.chg 2; neg.chg 1 |
| | H-region: length 1; peak value −0.15 |
| | PSG score: −4.55 |
| GvH: | von Heijne's method for signal seq. recognition |
| | GvH score (threshold: −2.1): −8.03 |
| | possible cleavage site: between 33 and 34 |

>>> Seems to have no N-terminal signal peptide

| ALOM: | Klein et al's method for TM region allocation | | | |
|---|---|---|---|---|
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 1.38 (at 31) | | | |
| | ALOM score: −1.70 (number of TMSs: 0) | | | |
| | MITDISC: discrimination of mitochondrial targeting seq | | | |
| | R content: | 2 | Hyd Moment(75): | 5.01 |
| | Hyd Moment(95): | 5.34 | G content: | 1 |
| | D/E content: | 2 | S/T content: | 0 |
| | Score: −5.94 | | | |

Gavel: prediction of cleavage sites for mitochondrial preseq

R-2 motif at 18 GRR|DV

NUCDISC: discrimination of nuclear localization signals

pat4: RRRH (3) at 342
pat4: RRHR (3) at 343
pat7: PKRQRAC (5) at 625
bipartite: none
content of basic residues: 10.6%
NLS Score: 0.33

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: found

KLPK at 623

RNA-binding motif: none
Actinin-type actin-binding motif:

| | type 1: none |
|---|---|
| | type 2: none |
| NMYR: | N-myristoylation pattern : none |

Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LGSTVNILVTRLILLTEDQPTL at 32

none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | Prediction: nuclear |
| | Reliability: 76.7 |
| COIL: | Lupas's algorithm to detect coiled-coil regions |
| | total: 0 residues |

--------------------------
Final Results (k = 9/23):

95.7%: nuclear
4.3%: cytoplasmic

>> prediction for CG51213-04 is nuc (k = 23)

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU97888 | Human aggrecanase protein #2 - | 1 . . . 855 | 852/855 (99%) | 0.0 |
| | Homo sapiens, 1104 aa. | 250 . . . 1103 | 852/855 (99%) | |
| | [WO200234895-A2, 02-MAY- | | | |
| | 2002] | | | |
| AAB74945 | Human ADAM type metal | 1 . . . 855 | 852/855 (99%) | 0.0 |
| | protease MDTS2 protein SEQ ID | 250 . . . 1103 | 852/855 (99%) | |
| | NO: 10 - Homo sapiens, 1103 aa. | | | |
| | [JP2001008687-A, 16 Jan. 2001] | | | |
| AAU72890 | Human metalloprotease partial | 1 . . . 855 | 851/855 (99%) | 0.0 |
| | protein sequence #2 - Homo | 250 . . . 1103 | 852/855 (99%) | |
| | sapiens, | | | |
| | 1103 aa. [WO200183782- | | | |
| | A2, 08-NOV-2001] | | | |
| ABG76505 | DNA encoding protein | 1 . . . 855 | 851/855 (99%) | 0.0 |
| | modification and maintenance | 250 . . . 1103 | 852/855 (99%) | |
| | molecule #9 - Homo sapiens, 1103 | | | |
| | aa. [WO200260942-A2, 08-AUG- | | | |
| | 2002] | | | |
| AAB47719 | ADAMTS-E - Homo sapiens, | 1 . . . 855 | 852/856 (99%) | 0.0 |
| | 1104 aa. [EP1149903-A1, 31- | 250 . . . 1104 | 852/856 (99%) | |
| | OCT-2001] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H324 | ADAMTS-10 precursor (EC | 1 . . . 855 | 850/855 (99%) | 0.0 |
| | 3.4.24.-) (A disintegrin and | 224 . . . 1077 | 851/855 (99%) | |
| | metalloproteinase with | | | |
| | thrombospondin motifs 10) | | | |
| | (ADAM-TS 10) (ADAM-TS10) - | | | |
| | Homo sapiens (Human), 1077 aa | | | |
| | (fragment). | | | |
| Q8CG28 | Zinc metalloendopeptidase - Mus | 1 . . . 854 | 809/855 (94%) | 0.0 |
| | musculus (Mouse), 1070 aa. | 216 . . . 1069 | 824/855 (95%) | |
| CAD20434 | Sequence 8 from Patent | 1 . . . 796 | 793/796 (99%) | 0.0 |
| | WO0188156 - Homo sapiens | 250 . . . 1044 | 793/796 (99%) | |
| | (Human), 1044 aa (fragment). | | | |
| CAC37777 | Sequence 11 from Patent | 246 . . . 797 | 550/552 (99%) | 0.0 |
| | WO0123561 - Homo sapiens | 83 . . . 634 | 550/552 (99%) | |
| | (Human), 634 aa (fragment). | | | |
| CAD20435 | Sequence 11 from Patent | 1 . . . 557 | 552/557 (99%) | 0.0 |
| | WO0188156 - Homo sapiens | 250 . . . 805 | 552/557 (99%) | |
| | (Human), 814 aa. | | | |

PFam analysis indicates that the NOV17a protein contains the domains shown in the Table 17F.

TABLE 17F
Domain Analysis of NOV17a

| | | Identities/ | |
|---|---|---|---|
| | NOV17a | Similarities | |
| Pfam | Match | for the Matched | Expect |
| Domain | Region | Matched Region | Value |
| Reprolysin | 2 . . . 209 | 59/235 (25%) | 1.4e-09 |
| | | 151/235 (64%) | |
| tsp_1 | 303 . . . 353 | 21/53 (40%) | 2.8e-07 |
| | | 37/53 (70%) | |
| tsp_1 | 581 . . . 636 | 11/59 (19%) | 0.056 |
| | | 38/59 (64%) | |
| tsp_1 | 640 . . . 696 | 14/63 (22%) | 0.13 |
| | | 38/63 (60%) | |
| tsp_1 | 698 . . . 754 | 16/57 (28%) | 0.43 |
| | | 34/57 (60%) | |
| tsp_1 | 759 . . . 809 | 17/55 (31%) | 0.2 |
| | | 34/55 (62%) | |

### Example 18

The NOV18 clone was analyzed, and the nucleotide and encoded polypeptide sequences are shown in Table 18A.

TABLE 18A
NOV18 Sequence Analysis

| | | |
|---|---|---|
| NOV18a, CG51448-05 | SEQ ID NO: 211 | 1762 bp |
| DNA Sequence | ORF Start: ATG at 197 | ORF Stop: TGA at 1760 |

TTTGAGTTAGACAAGCAGCAGCACACGCCTCCCTACCTCATGGCGACAGAAAATGGAGCAGTTGAGCT
GGGAATTCAGAACCCATCAACAGACAAGGCACCTAAAGGTCCCACAGGTGAAAGACCCCTGGCTGCAG
GGAAAGACCCTGGCCCCCCAGACCCAAAGAAAGCTCCGGATCCACCCACCCTGAAGAAAGATGCCAAA
GCCCCTGCCTCAGAGAAAGGGGATGGTACCCTGGCCCAACCCTCAACTAGCAGCCCTGCCCCAGCAGA
CTGCGACACCTGAGACCAGCGTCAAGAAGCCCAAGGCTGAGCAGGGAGCCTCAGGCAGCCAGGATCCT
GGAAAGCCCAGGGTGGGCAAGAAGGCAGCAGAGGGCCAAGCAGCAGCCAGGAGGGGCTCACCTGCCTT
TCTGCATAGCCCCAGCTGTCCTGCCATCATCTCCAGTTCTGAGAAGCTGCTGGCCAAGAAGCCCCCAA
GCGAGGCATCAGAGCTCACCTTTGAAGGGGTGCCCATGACCCACAGCCCCACGGATCCCAGGCCAGCC
AAGGCAGAAGAAGGAAAGAACATCCTGGCAGAGAGCCAGAAGGAAGTGGGAGAGAAAACCCCAGGCCA
GGCTGGCCAGGCTAAGATGCAAGGGGACACCTCGAGGGGGATTGAGTTCCAGGCTGTTCCCTCAGAGA
AATCCGAGGTGGGGCAGGCCCTCTGTCTCACAGCCAGGGAGGAGGACTGCTTCCAGATTTTGGATGAT
TGCCCGCCACCTCCGGCCCCCTTCCCTCACCGCATGGTGGAGCTGAGGACCGGGAATGTCAGCAGTGA
ATTCAGTATGAACTCCAAGGAGGCGCTCGGAGGTGGCAAGTTTGGGGCAGTCTGTACCTGCATGGAGA
AAGCCACAGGCCTCAAGCTGGCAGCCAAGGTCATCAAGAAACAGACTCCCAAAGACAAGGAAATGGTG
TTGCTGGAGATTGAGGTCATGAACCAGCTGAACCACCGCAATCTGATCCAGCTGTATGCAGCCATCGA
GACTCCGCATGAGATCGTCCTGTTCATGGAGTACATCGAGGGCGGAGAGCTCTTCGAGAGGATTGTGG
ATGAGGACTACCATCTGACCGAGGTGGACACCATGGTGTTTGTCAGGCAGATCTGTGACGGGATCCTC
TTCATGCACAAGATGAGGGTTTTGCACCTGGACCTCAAGCCAGAGAACATCCTGTGTGTCAACACCAC
CGGGCATTTGGTGAAGATCATTGACTTTGGCCTGGCACGGAGGTATAACCCCAACGAGAAGCTGAAGG
TGAACTTTGGGACCCCAGAGTTCCTGTCACCTGAGGTGGTGAATTATGACCAAATCTCCGATAAGACA
GACATGTGGAGTATGGGGGTGATCACCTACATGCTGCTGAGCGGCCTCTCCCCCTTCCTGGGAGATGA
TGACACAGAGACCCTAAACAACGTTCTATCTGGCAACTGGTACTTTGATGAAGAGACCTTTGAGGCCG
TATCAGACGAGGCCAAAGACTTTGTCTCCAACCTCATCGTCAAGGACCAGAGGGCCCGGATGAACGCT
GCCCAGTGTCTCGCCCATCCCTGGCTCAACAACCTGGCGGAGAAAGCCAAACGCTGTAACCGACGCCT
TAAGTCCCAGATCTTGCTTAAGAAATACCTCATGAAGAGGCGCTGGAAGAAAAACTTCATTGCTGTCA
GCGCTGCCAACCGCTTCAAGAAGATCAGCAGTTCGGGGGCACTGATGGCTCTGGGGGTCTGA

| | | | |
|---|---|---|---|
| NOV18a,CG51448-05 | SEQ ID NO: 212 | 521 aa | MW at 57751.9kD |
| Protein Sequence | | | |

MPKPLPQRKGMVPWPNPQLAALPQQTATPETSVKKPKAEQGASGSQDPGKPRVGKKAAEGQAAARRGS
PAFLHSPSCPAIISSSEKLLAKKPPSEASELTFEGVPMTHSPTDPRPAKAEEGKMILAESQKEVGEKT
PGQAGQAKMQGDTSRGIEFQAVPSEKSEVGQALCLTAREEDCFQILDDCPPPPAPFPHRMVELRTGNV
SSEFSNNSKEALGGGKFGAVCTCMEKATGLKLAAKVIKKQTPKDKEMVLLEIEVMNQLNHRNLIQLYA
AIETPHEIVLFMBYIEGGELFERIVDEDYHLTEVDTNVFVRQICDGILFMHKMRVLHLDLKPENILCV
NTTGHLVKIIDFGLARRYNPNEKLKVNFGTPEFLSPEVVNYDQISDKTDMWSMGVITYMLLSGLSPFL
GDDDTETLNNVLSGNWYFDEETFEAVSDEAKDFVSNLIVKDQRARMNAAQCLAHPWLNNLAEKAKRCN
RRLKSQILLKKYLMKRRWKKNFIAVSAANRFKKISSSGALMALGV

| | | |
|---|---|---|
| NOV18b, CG51448-01 | SEQ ID NO: 213 | 1788 bp |
| DNA Sequence | ORF Start: ATG at 1 | ORF Stop: TGA at 1786 |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18b, CG51448-01 | SEQ ID NO: 214 | 595 aa | MW at 64501.9kD |
| Protein Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIEDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARNNAAQOLAHPWLNNLAE
KAKRCNRRLKSQILLKKYLMKRRWKKNFIAVSAVSAANRFKKISSSGALMALGV

| | | |
|---|---|---|
| NOV18c, 274051198 | SEQ ID NO: 215 | 787 bp |
| DNA Sequence | ORF Start: at 2 | ORF Stop: end of sequence |

CACCAAGCTTATGAACTCCAAGGAGGCGCTCGGAGGGGGCAAGTTTGGGGCAGTCTGTACCTGCATGG
AGAAAGCCACAGGCCTCAAGCTGGCAGCCAAGGTCATCAAGAAACAGACTCCCAAAGACAAGGAAATG
GTGTTGCTGGAGATTGAGGTCATGAACCAGCTGAACCACCGCAATCTGATCCAGCTGTATGCAGCCAT
CGAGACTCCGCATGAGATCGTCCTGTTCATGGAGTACATCGAGGGCGGAGAGCTCTTCGAGAGGATTG
TGGATGAGGACTACCATCTGACCGAGGTGGACACCATGGTGTTTGTCAGGCAGATCTGTGACGGGATC
CTCTTCATGCACAAGATGAGGGTTTTGCACCTGGACCTCAAGCCAGAGAACATCCTGTGTGTCAACAC
CACCGGGCATTTGGTGAAGATCATTGACTTTGGCCTGGCACGGAGGTATAACCCCAACGAGAAGCTGA
AGGTGAACTTTGGGACCCCAGAGTTCCTGTCACCTGAGGTGGTGAATTATGACCAAATCTCCGATAAG
ACAGACATGTGGAGTATGGGGGTGATCACCTACATGCTGCTGAGCGGCCTCTCCCCCTTCCTGGGAGA
TGATGACACAGAGACCCTAAACAACGTTCTATCTGGCAACTGGTACTTTGATGAAGAGACCTTTGAGG
CCGTATCAGACGAGGCCAAAGACTTTGTCTCCAACCTCATCGTCAAGGACCAGAGGGCCCGGATGAAC
GCTGCCCAGTGTCTCGCCCATCCCTGGCTCGTCGACGGC

| | | | |
|---|---|---|---|
| NOV18c, 274051198 | SEQ ID NO: 216 | 262 aa | MW at 29771.1kD |
| Protein Sequence | | | |

TKLMNSKEALGGGKFGAVCTCMEKATGLKLAAKVIKKQTPKDKEMVLLEIEVMNQLNHRNLIQLYAAI
ETPHEIVLFMEYIEGGELFERIVDEDYHLTEVDTMVFVRQICDGILFNHKMRVLHLDLKPENILCVNT
TGHLVKIIDFGLARRYNPNEKLKVNFGTPEFLSPEVVNYDQISDKTDMWSMGVITYMLLSGLSPFLGD
DDTETLNNVLSGNWYFDEETFEAVSDEAKDFVSNLIVKDQRARMNAAQCLAHPWLVDG

| | | |
|---|---|---|
| NOV18d, 274051170 | SEQ ID NO: 217 | 1813 bp |
| DNA Sequence | ORF Start: at 2 | ORF Stop: end of sequence |

CACCAAGCTTCCCACCATGGCGACAGAAAATGGAGCAGTTGAGCTGGGAATTCAGAACCCATCAACAG
ACAAGGCACCTAAAGGTCCCACAGGTGAAAGACCCCTGGCTGCAGGGAAAGACCCTGGCCCCCCAGAC
CCAAAGAAAGCTCCGGATCCACCCACCCTGAAGAAAGATGCCAAAGCCCCTGCCTCAGAGAAAGGGGA
TGGTACCCTGGCCCAACCCTCAACTAGCAGCCAAGGCCCCAAAGGAGAGGGTGACAGGGGCGGGGGGC
CCGCGGAGGGCAGTGCTGGGCCCCCGGCAGCCCTGCCCCAGCAGACTGCGACACCTGAGACCAGCGTC
AAGAAGCCCAAGGCTGAGCAGGGAGCCTCAGGCAGCCAGGATCCTGGAAAGCCCAGGGTGGGCAAGAA
GGCAGCAGAGGGCCAAGCAGCAGCCAGGAGGGGCTCACCTGCCTTTCTGCATAGCCCCAGCTGTCCTG
CCATCATCTCCAGTTCTGAGAAGCTGCTGGCCAAGAAGCCCCCAAGCGAGGCATCAGAGCTCACCTTT
GAAGGGGTGCCCATGACCCACAGCCCCACGGATCCCAGGCCAGCCAAGGCAGAAGAAGGAAAGAACAT
CCTGGCAGAGAGCCAGAAGGAAGTGGGAGAGAAAACCCCAGGCCAGGCTGGCCAGGCTAAGATGCAAG
GGGACACCTCGAGGGGGATTGAGTTCCAGGCTGTTCCCTCAGAGAAATCCGAGGTGGGGCAGGCCCTC
TGTCTCACAGCCAGGGAGGAGGACTGCTTCCAGATTTTGGATGATTGCCCGCCACCTCCGGCCCCCTT
CCCTCACCGCATGGTGGAGCTGAGGACCGGGAATGTCAGCAGTGAATTCAGTATGAACTCCAAGGAGG
CGCTCGGAGGTGGCAAGTTTGGGGCAGTCTGTACCTGCATGGAGAAAGCCACAGGCCTCAAGCTGGCA
GCCAAGGTCATCAAGAAACAGACTCCCAAAGACAAGGAAATGGTGTTGCTGGAGATTGAGGTCATGAA
CCAGCTGAACCACCGCAATCTGATCCAGCTGTATGCAGCCATCGAGACTCCGCATGAGATCGTCCTGT
TCATGGAGTACATCGAGGGCGGAGAGCTCTTCGAGAGGATTGTGGATGAGGACTACCATCTGACCGAG
GTGGACACCATGGTGTTTGTCAGGCAGATCTGTGACGGGATCCTCTTCATGCACAAGATGAGGGTTTT
GCACCTGGACCTCAAGCCAGAGAACATCCTGTGTGTCAACACCACCGGGCATTTGGTGAAGATCATTG
ACTTTGGCCTGGCACGGAGGTATAACCCCAACGAGAAGCTGAAGGTGAACTTTGGGACCCCAGAGTTC
CTGTCACCTGAGGTGGTGAATTATGACCAAATCTCCGATAAGACAGACATGTGGAGTATGGGGGTGAT
CACCTACATGCTGCTGAGCGGCCTCTCCCCCTTCCTGGGAGATGATGACACAGAGACCCTAAACAACG
TTCTATCTGGCAACTGGTACTTTGATGAAGAGACCTTTGAGGCCGTATCAGACGAGGCCAAAGACTTT
GTCTCCAACCTCATCGTCAAGGACCAGAGGGCCCGGATGAACGCTGCCCAGTGTCTCGCCCATCCCTG
GCTCAACAACCTGGCGGAGAAAGCCAAACGCTGTAACCGACGCCTTAAGTCCCAGATCTTGCTTAAGA
AATACCTCATGAAGAGGCGCTGGAAGAAAAACTTCATTGCTGTCAGCGCTGCCAACCGCTTCAAGAAG
ATCAGCAGCTCGGGGGCACTGATGGCTCTGGGGGTCGTCGACGGC

| | | | |
|---|---|---|---|
| NOV18d, 274051170 | SEQ ID NO: 218 | 604 aa | MW at 65496.1kD |
| Protein Sequence | | | |

TKLPTMATENGAVELGIQNPSTDKAPKGPTGERPLAAGKDPGPPDPKKAPDPPTLKKDAKAPASEKGD
GTLAQPSTSSQGPKGEGDRGGGPAEGSAGPPAALPQQTATPETSVKKPKAEQGASGSQDPGKPRVGKK
AAEGQAAARRGSPAFLHSPSCPAIISSSEKLLAKKPPSEASELTFEGVPMTHSPTDPRPAKAEEGKNI
LAESQKEVGEKTPGQAGQAKMQGDTSRGIEFQAVPSEKSEVGQALCLTAREEDCFQILDDCPPPPAPF
PHRMVELRTGNVSSEFSMNSKEALGGGKFGAVCTCMEKATGLKLAAKVIKKQTPKDKEMVLLEIEVMN
QLNHRNLIQLYAAIETPHEIVLFMEYIEGGELFERIVDEDYHLTEVDTMVFVRQICDGILFMHKMRVL
HLDLKPENILCVNTTGHLVKIIDFGLARRYNPNEKLKVNFGTPEFLSPEVVNYDQISDKTDMWSMGVI
TYMLLSGLSPFLGDDDTETLNNVLSGNWYFDEETFEAVSDEAKDFVSNLIVKDQRARMNAAQCLAHPW
LNNLAEKAKRCNRRLKSQILLKKYLMKRRWKKNFIAVSAANRFKKISSSGALMALGVVDG

| | | |
|---|---|---|
| NOV18e, GG51448-02 | SEQ ID NO: 219 | 2010 bp |
| DNA Sequence | ORF Start: ATG at 40 | ORF Stop: TGA at 1828 |

TTTGAGTTAGACAAGCAGCAGCACACGCCTCCCTACCTCATGGCGACAGAAAATGGAGCAGTTGAGCT
GGGAATTCAGAACCCATCAACAGACAAGGCACCTAAAGGTCCCACAGGTGAAAGACCCCTGGCTGCAG
GGAAAGACCCTGGCCCCCCAGACCCAAAGAAAGCTCCGGATCCACCCACCCTGAAGAAAGATGCCAAA
GCCCCTGCCTCAGAGAAAGGGGATGGTACCCTGGCCCAACCCTCAACTAGCAGCCAAGGCCCCAAAGG
AGAGGGTGACAGGGGCGGGGGGCCCGCGGAGGGCAGTGCTGGGCCCCCGGCAGCCCTGCCCCAGCAGA
CTGCGACACCTGAGACCAGCGTCAAGAAGCCCAAGGCTGAGCAGGGAGCCTCAGGCAGCCAGGATCCT
GGAAAGCCCAGGGTGGGCAAGAAGGCAGCAGAGGGCCAAGCAGCAGCCAGGAGGGGCTCACCTGCCTT
TCTGCATAGCCCCAGCTGTCCTGCCATCATCTCCAGTTCTGAGAAGCTGCTGGCCAAGAAGCCCCCAA
GCGAGGCATCAGAGCTCACCTTTGAAGGGGTGCCCATGACCCACAGCCCCACGGATCCCAGGCCAGCC
AAGGCAGAAGAAGGAAAGAACATCCTGGCAGAGAGCCAGAAGGAAGTGGGAGAGAAAACCCCAGGCCA
GGCTGGCCAGGCTAAGATGCAAGGGGACACCTCGAGGGGGATTGAGTTCCAGGCTGTTCCCTCAGAGA
AATCCGAGGTGGGGCAGGCCCTCTGTCTCACAGCCAGGGAGGAGGACTGCTTCCAGATTTTGGATGAT
TGCCCGCCACCTCCGGCCCCCTTCCCTCACCGCATGGTGGAGCTGAGGACCGGGAATGTCAGCAGTGA
ATTCAGTATGAACTCCAAGGAGGCGCTCGGAGGTGGCAAGTTTGGGGCAGTCTGTACCTGCATGGAGA
AAGCCACAGGCCTCAAGCTGGCAGCCAAGGTCATCAAGAAACAGACTCCCAAAGACAAGGAAATGGTG
TTGCTGGAGATTGAGGTCATGAACCAGCTGAACCACCGCAATCTGATCCAGCTGTATGCAGCCATCGA
GACTCCGCATGAGATCGTCCTGTTCATGGAGTACATCGAGGGCGGAGAGCTCTTCGAGAGGATTGTGG
ATGAGGACTACCATCTGACCGAGGTGGACACCATGGTGTTTGTCAGGCAGATCTGTGACGGGATCCTC
TTCATGCACAAGATGAGGGTTTTGCACCTGGACCTCAAGCCAGAGAACATCCTGTGTGTCAACACCAC
CGGGCATTTGGTGAAGATCATTGACTTTGGCCTGGCACGGAGGTATAACCCCAACGAGAAGCTGAAGG
TGAACTTTGGGACCCCAGAGTTCCTGTCACCTGAGGCGGTGAATTATGACCAAATCTCCGATAAGACA
GACATGTGGAGTATGGGGGTGATCACCTACATGCTGCTGAGCGGCCTCTCCCCCTTCCTGGGAGATGA
TGACACAGAGACCCTAAACAACGTTCTATCTGGCAACTGGTACTTTGATGAAGAGACCTTTGAGGCCG
TATCAGACGAGGCCAAAGACTTTGTCTCCAACCTCATCGTCAAGGACCAGAGGGCCCGGATGAACGCT
GCCCAGTGTCTCGCCCATCCCTGGCTCAACAACCTGGCGGAGAAAGCCAAACGCTGTAACCGACGCCT
TAAGTCCCAGATCTTGCTTAAGAAATACCTCATGAAGAGGCGCTGGAAGAAAAACTTCATTGCTGTCA
GCGCTGCCAACCGCTTCAAGAAGATCAGCAGCTCGGGGGCACTGATGGCTCTGGGGGTCTGAGCCCTG
GGCGCAGCTGAAGCCTGGACGCAGCCACACAGTGGCCGGGGCTGAAGCCACACAGCCCAGAAGGCCAG
AAAAGGCAGCCAGATCCCCAGGGCAGCCTCGTTAGGACAAGGCTGTGCCAGGCTGGGAGGCTCGGGGC
TCCCCACGCCCCCATGCAGTGACCGCTTCCCCGATGTG

| | | | |
|---|---|---|---|
| NOV18e, GG51448-02 | SEQ ID NO: 220 | 596 aa | MW at 64656.1kD |
| Protein Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEYIEGGELFERIVDEDYHLTEVDTMVFVRQICDGILFMNKMRVLHLDLK
PENILCVNTTGHLVKIIDFGLARRYNPNEKLKVNFGTPEFLSPEAVNYDQISDKTDMWSMGVITYMLL
SGLSPFLGDDDTETLNNVLSGNWYFDEETFEAVSDEAKDFVSNLIVKDQRARMNAAQCLAHPWLNNLA
EKAKRCNRRLKSQILLKKYLMKRRWKKNFIAVSAANRFKKISSSGALMALGV

| | | |
|---|---|---|
| NOV18f, CG51448-03 | SEQ ID NO: 221 | 1839 bp |
| DNA Sequence | ORF Start: ATG at 49 | ORF Stop: TGA at 1837 |

CTAGAAAGACTTGAGTTAGACAAGCAGCAGCACACGCCTCCCTACCTCATGGCGACAGAAAATGGAGC
AGTTGAGCTGGGAATTCAGAACCCATCAACAGACAAGGCACCTAAAGGTCCCACAGGTGAAAGACCCC
TGGCTGCAGGGAAAGACCCTGGCCCCCCAGACCCAAAGAAAGCTCCGGATCCACCCACCCTGAAGAAA
GATGCCAAAGCCCCTGCCTCAGAGAAAGGGGATGGTACCCTGGCCCAACCCTCAACTAGCAGCCAAGG
CCCCAAAGGAGAGGGTGACAGGGGCGGGGGGCCCGCGGAGGGCAGTGCTGGGCCCCCGGCAGCCCTGC
CCCAGCAGACTGCGACACCTGAGACCAGCGTCAAGAAGCCCAAGGCTGAGCAGGGAGCCTCAGGCAGC
CAGGATCCTGGAAAGCCCAGGGTGGGCAAGAAGGCAGCAGAGGGCCAAGCAGCAGCCAGGAGGGGCTC
ACCTGCCTTTCTGCATAGCCCCAGCTGTCCTGCCATCATCTCCAGTTCTGAGAAGCTGCTGGCCAAGA
AGCCCCCAAGCGAGGCATCAGAGCTCACCTTTGAAGGGGTGCCCATGACCCACAGCCCCACGGATCCC
AGGCCAGCCAAGGCAGAAGAAGGAAAGAACATCCTGGCAGAGAGCCAGAAGGAAGTGGGAGAGAAAAC
CCCAGGCCAGGCTGGCCAGGCTAAGATGCAAGGGGACACCTCGAGGGGGATTGAGTTCCAGGCTGTTC
CCTCAGAGAAATCCGAGGTGGGGCAGGCCCTCTGTCTCACAGCCAGGGAGGAGGACTGCTTCCAGATT
TTGGATGATTGCCCGCCACCTCCGGCCCCCTTCCCTCACCGCATGGTGGAGCTGAGGACCGGGAATGT
CAGCAGTGAATTCAGTATGAACTCCAAGGAGGCGCTCGGAGGTGGCAAGTTTGGGGCAGTCTGTACCT
GCATGGAGAAAGCCACAGGCCTCAAGCTGGCAGCCAAGGTCATCAAGAAACAGACTCCCAAAGACAAG
GAAATGGTGTTGCTGGAGATTGAGGTCATGAACCAGCTGAACCACCGCAATCTGATCCAGCTGTATGC
AGCCATCGAGACTCCGCATGAGATCGTCCTGTTCATGGAGTACATCGAGGGCGGAGAGCTCTTCGAGA
GGATTGTGGATGAGGACTACCATCTGACCGAGGTGGACACCATGGTGTTTGTCAGGCAGATCTGTGAC
GGGATCCTCTTCATGCACAAGATGAGGGTTTTGCACCTGGACCTCAAGCCAGAGAACATCCTGTGTGT
CAACACCACCGGGCATTTGGTGAAGATCATTGACTTTGGCCTGGCACGGAGGTATAACCCCAACGAGA
AGCTGAAGGTGAACTTTGGGACCCCAGAGTTCCTGTCACCTGAGGCGGTGAATTATGACCAAATCTCC
GATAAGACAGACATGTGGAGTATGGGGGTGATCACCTACATGCTGCTGAGCGGCCTCTCCCCCTTCCT
GGGAGATGATGACACAGAGACCCTAAACAACGTTCTATCTGGCAACTGGTACTTTGATGAAGAGACCT
TTGAGGCCGTATCAGACGAGGCCAAAGACTTTGTCTCCAACCTCATCGTCAAGGACCAGAGGGCCCGG
ATGAACGCTGCCCAGTGTCTCGCCCATCCCTGGCTCAACAACCTGGCGGAGAAAGCCAAACGCTGTAA
CCGACGCCTTAAGTCCCAGATCTTGCTTAAGAAATACCTCATGAAGAGGCGCTGGAAGAAAAACTTCA
TTGCTGTCAGCGCTGCCAACCGCTTCAAGAAGATCAGCAGCTCGGGGGCACTGATGGCTCTGGGGGTC
TGA

| | | | |
|---|---|---|---|
| NOV18f, CG51448-03 | SEQ ID NO: 222 | 596 aa | MW at 64656.1kD |
| Protein Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEYIEGGELFERIVDEDYHLTEVDTMVFVRQICDGILFMHKMRVLHLDLK
PENILCVNTTGHLVKIIDFGLARRYNPNEKLKVNFGTPEFLSPEAVNYDQISDKTDMWSMGVITYMLL
SGLSPFLGDDDTETLNNVLSGNWYFDEETFEAVSDEAKDFVSNLIVKDQRARMNAAQCLAHPWLNNLA
EKAKRCNRRLKSQILLKKYLMKRRWKKNFIAVSAANRFKKISSSGALMALGV

| | | |
|---|---|---|
| NOV18g, GG51448-04 | SEQ ID NO: 223 | 2558 bp |
| DNA Sequence | ORF Start: ATG at 164 | ORF Stop: TGA at 1949 |

CTTTGCTCCAGGTACCTCTCTCCCCTCAGTTAGCAGGCCTCGGCTTCCTGTCTCACTGCAGCCAGACG
AGAGGGGAAATTGGACAGCCTGACACACTCCACTCTTGTTTCTGCAGCTAGAAAGACTTGAGTTAGAC
AAGCAGCAGCACACGCCTCCCTACCTCATGGCGACAGAAAATGGAGCAGTTGAGCTGGGAATTCAGAA
CCCATCAACAGACAAGGCACCTAAAGGTCCCACAGGTGAAAGACCCCTGGCTGCAGGGAAAGACCCTG
GCCCCCCAGACCCAAAGAAAGCTCCGGATCCACCCACCCTGAAGAAAGATGCCAAAGCCCCTGCCTCA
GAGAAAGGGGATGGTACCCTGGCCCAACCCTCAACTAGCAGCCAAGGCCCCAAAGGAGAGGGTGACAG
GGGCGGGGGGCCCGCGGAGGGCAGTGCTGGGCCCCCGGCAGCCCTGCCCCAGCAGACTGCGACACCTG
AGACCAGCGTCAAGAAGCCCAAGGCTGAGCAGGGAGCCTCAGGCAGCCAGGATCCTGGAAAGCCCAGG
GTGGGCAAGAAGGCAGCAGAGGGCCAAGCAGCAGCCAGGAGGGGCTCACCTGCCTTTCTGCATAGCCC
CAGCTGTCCCGCCATCATCTCCAGTTCTGAGAAGCTGCTGGCCAAGAAGCCCCCAAGCGAGGCATCAG
AGCTCACCTTTGAAGGGGTGCCCATGACCCACAGCCCCACGGATCCCAGGTCGGCCAAGGCAGAAGAA
GGAAAGAACATCCTGGCAGAGAGCCAGAAGGAAGTGGGAGAGAAAACCCCAGGCCAGGCTGGCCAGGC
TAAGATGCAAGGGGACACCTCGAGGGGGATTGAGTTCCAGGCTGTTCCCTCAGAGAAATCCGAGGTGG
GGCAGGCCCTCTGTCTCACAGCCAGGGAGGAGGACTGCTTCCAGATTTTGGATGATTGCCCGCCACCT
CCGGCCCCCTTCCCTCACCGCATGGTGGAGCTGAGGACCGGGAATGTCAGCAGTGAATTCAGTATGAA
CTCCAAGGAGGCGCTCGGAGGGGGCAAGTTTGGGGCAGTCTGTACCTGCATGGAGAAAGCCACAGGCC
TCAAGCTGGCAGCCAAGGTCATCAAGAAACAGACTCCCAAAGACAAGGAAATGGTGTTGCTGGAGATT
GAGGTCATGAACCAGCTGAACCACCGCAATCTGATCCAGCTGTATGCAGCCATCGAGACTCCGCATGA
GATCGTCCTGTTCATGGAGATCGAGGGCGGAGAGCTCTTCGAGAGGATTGTGGATGAGGACTACCATC
TGACCGAGGTGGACACCATGGTGTTTGTCAGGCAGATCTGTGACGGGATCCTCTTGATGCACAAGATG
AGGGTTTTGCACCTGGACCTCAAGCCAGAGAACATCCTGTGTGTCAACACCACCGGGCATTTGGTGAA
GATCATTGACTTTGGCCTGGCACGGAGGTATAACCCCAACGAGAAGCTGAAGGTGAACTTTGGGACCC
CAGAGTTCCTGTCACCTGAGGTGGTGAATTATGACCAAATCTCCGATAAGACAGACATGTGGAGTATG
GGGGTGATCACCTACATGCTGCTGAGCGGCCTCTCCCCCTTCCTGGGAGATGATGACACAGAGACCCT
AAACAACGTTCTATCTGGCAACTGGTACTTTGATGAAGAGACCTTTGAGGCCGTATCAGACGAGGCCA
AAGACTTTGTCTCCAACCTCATCGTCAAGGACCAGAGGGCCCGGATGAACGCTGCCCAGTGTCTCGCC
CATCCCTGGCTCAACAACCTGGCGGAGAAAGCCAAACGCTGTAACCGACGCCTTAAGTCCCAGATCTT
GCTTAAGAAATACCTCATGAAGAGGCGCTGGAAGAAAAACTTCATTGCTGTCAGCGCTGCCAACCGCT
TCAAGAAGATCAGCAGCTCGGGGGCACTGATGGCTCTGGGGGTCTGAGCCCTGGGCGCAGCTGAAGCC
TGGACGCAGCCACACAGTGGCCGGGGCTGAAGCCACACAGCCCAGAAGGCCAGAAAAGGCAGCCAGAT
CCCCAGGGCAGCCTCGTTAGGACAAGGCTGTGCCAGGCTGGGAGGCTCGGGGCTCCCCACGCCCCCAT
GCAGTGACCGCTTCCCCGATGTGAGCCGCCTCGGAGTGTGGCCTGGATCCATCCTGCTAGCACCTCCC
CAGACAGGGCTCCAGCCTGTCGGCCACACCCCAGACTCCAGGCCCCCGTTGAAGCCGCTCCCGGTTCC
CTCCCCAGCTCCTCGTCTTTGAACTGCCGCCGCCGTGGTGACCCCTGCTTTGCCCCACTGGGAGAGTC
CTTAGCCTGGGCCTCCTCCTAGCTGGAGTGCCATGGCTGGGGGGTCTCAGCATGTAGGGCTTCTGTGG
TTGTGGATGGGAGGCTCCTGGTGGGGCAGAAAGGCTGCAACGCTGATTCCTAAGGCCCAGCTGCCAGG
GAAGACAGAGCAGGCTTTGTGAGAGAGGACCTCCATGCCCCCGCCACCTCCCCACTCCAGCAGATAAG
GCCGAGCCCACACCATCTGGCCCAGGCTGGCCCCCACCACCT

| | | | |
|---|---|---|---|
| NOV18g, CG51448-04 | SEQ ID NO: 224 | 595 aa | MW at 64476.9kD |
| Protein Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRSAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILLMHKMRVLHLDLKP
ENILCVNTTGHLVKIIDFGLARRYNPNEKLKVNFGTPEFLSPEVVNYDQISDKTDMWSMGVITYMLLS
GLSPFLGDDDTETLNNVLSGNWYFDEETFEAVSDEAKDFVSNLIVKDQRARMNAAQCLAHPWLNNLAE
KAKRCNRRLKSQILLKKYLMKRRWKKNFIAVSAANRFKKISSSGALMALGV

| | | |
|---|---|---|
| NOV18h, SNP13375535 of | SEQ ID NO: 225 | 1788 bp |
| CG51448-01, DNA Sequence | ORF Start: ATG at 1 | ORF Stop: TGA at 1786 |
| | SNP Pos: 102 | SNP Change: A to G |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAGGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18h, SNP13375535 of | SEQ ID NO: 226 | 595 aa | MW at 64501.9kD |
| CG51448-01, Protein | SNP Pos: 34 | | SNP Change: Lys to Lys |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARNNAAQCLAHPWLNNLAE
KAKRCNRRLKSQILLKKYLMKRRWKKNFIAVSAANRFKKISSSGALMALGV

| | | |
|---|---|---|
| NOV18i, SNP13375536 of | SEQ ID NO: 227 | 1788 bp |
| CG51448-01, DNA Sequence | ORF Start: ATG at 1 | ORF Stop: TGA at 1786 |
| | SNP Pos: 287 | SNP Change: C to T |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGTCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18i, SNP13375536 of | SEQ ID NO: 228 | 595 aa | MW at 64530.0kD |
| CG51448-01, Protein | SNP Pos: 96 | | SNP Change: Ala to Val |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAVLPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKENVLLEIEVMNQLNHR
NLIQLYAAIETPHEIVLFMEEEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARMNAAQCLAHPWLNNLAE
KAKRCNRRLKSQILLKKYLMKRRWKKNFIAVSAANRFKKISSSGALMALGV

| | | |
|---|---|---|
| NOV18j, SNP13375537 of | SEQ ID NO: 229 | 1788 bp |
| CG51448-01, DNA Sequence | ORF Start: ATG at 1 | ORF Stop: TGA at 1786 |
| | SNP Pos: 350 | SNP Change: C to T |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGTCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18j, SNP13375537 of | SEQ ID NO: 230 | 595 aa | MW at 64530.0kD |
| CG51448-01, Protein | SNP Pos: 117 | | SNP Change: Ala to Val |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGVSGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARMNAAQCLAHPWLNNLAE
KAKRCNRRLKSQILLKKYLMKRRWKKNFIAVSAANRFKKISSSGALMALGV

| | | |
|---|---|---|
| NOV18k, SNP13375538 of | SEQ ID NO: 231 | 1788 bp |
| CG51448-01, DNA Sequence | ORF Start: ATG at 1 | ORF Stop: TGA at 1786 |
| | SNP Pos: 1249 | SNP Change: A to T |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCTCCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18k, SNP13375538 of | SEQ ID NO: 232 | 595 aa | MW at 64487.9kD |
| CG51448-01, Protein | SNP Pos: 417 | | SNP Change: Thr to Ser |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRNV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTSGHLVKIIDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARMNAAQCLAHPWLNNLAE
KAKRCNRRLKSQILLKKYLMKRRWKKNFIAVSAANRFKKISSSGALMALGV

| | | |
|---|---|---|
| NOV18l, SNP13375539 of | SEQ ID NO: 233 | 1788 bp |
| CG51448-01, DNA Sequence | ORF Start: ATG at 1 | ORF Stop: TGA at 1786 |
| | SNP Pos: 1358 | INP Change: T to C |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGCGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18l, SN1P13375539 of | SEQ ID NO: 234 | 595 aa | MW at 64473.8kD |
| CG51448-01, Protein | SNP Pos: 453 | | SNP Change: Val to Ala |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKABEGKNILAESQ
KBVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTNVFVRQICDGILFMHKMRVLHLDLKP
ENILCVNTTGHLVKIIDFGLARRYHNPNEKLKVNFGTPEFLSPEAVNYDQISDKTDMWSMGVITYMLL
SGLSPFLGDDDTETLNNVLSGNWYFDEETFEAVSDEAKDFVSNLIVKDQARMNAAQCLAHPWLNNLAE
KAKRCNRRLKSQILLKKYLMKRRWKKNFIAVSAANRFKKISSSGALMALGV

| | | |
|---|---|---|
| NOV18m, SNP13375540 of | SEQ ID NO: 235 | 1788 bp |
| CG51448-01, DNA Sequence | ORF Start: ATG at 1 | ORF Stop: TGA at 1786 |
| | SNP Pos: 1391 | SNP Change: A to G |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGGCATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18m, SNP13375540 of | SEQ ID NO: 236 | 595 aa | MW at 64443.9kD |
| CG51448-01, Protein | SNP Pos: 464 | | SNP Change: Asp to Gly |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKMILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFNEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTGMWSMGVITYMLL
SGLSPFLGDDDTETLNNVLSGNWYFDEETFEAVSDEAKDFVSNLIVKDQARNNAAQCLAHPWLNNLAE
KAKRCNRRLKSQILLKKYLMKRRWKKNFIAVSAANRFKKISSSGALMALGV

| | | |
|---|---|---|
| NOV18n, SNP13375541 of | SEQ ID NO: 237 | 1788 bp |
| CG51448-01, DNA Sequence | ORF Start: ATG at 1 | ORF Stop: TGA at 1786 |
| | SNP Pos: 1511 | SNP Change: A to G |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGGGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18n, SNP13375541 of | SEQ ID NO: 238 | 595 aa | MW at 64429.8kD |
| CG51448-01, Protein | SNP Pos: 504 | | SNP Change: Glu to Gly |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLNNVLSGNWYFDEGTFEAVSDEAKDFVSNLIVKDQARMNAAQCLAHPWLNNLAE
KAKRCNRRLKSQILLKKYLMKRRWKKNFIAVSAANRFKKISSSGALMALGV

| | | |
|---|---|---|
| NOV18o, SNP13375542 of | SEQ ID NO: 239 | 1788 bp |
| CG51448-01, DNA Sequence | ORF Start: ATG at 1 | ORF Stop: TGA at 1786 |
| | SNP Pos: 1675 | SNP Change: C to T |

ATGGCGACAGAAAATGGAGCAGTTGAGCTGGGAATTCAGAACCCATCAACAGACAAGGCACCTAAAGG
TCCCACAGGTGAAAAGACCCCTGGCTGCAGGGAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTCACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGTTTAAGAAATACCTCATGAAGAGGCG
CTGGAAGAAAAACTTCATTGCTGTCAGCGCTGCCAACCGCTTCAAGAAGATCAGCAGCTCGGGGGCAC
TGATGGCTCTGGGGGTCTGA

| | | | |
|---|---|---|---|
| NOV18o, SNP13375542 of | SEQ ID NO: 240 | 595 aa | MW at 64535.9kD |
| CG51448-01, Protein | SNP Pos: 559 | | SNP Change: Leu to Phe |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEOGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSMNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLNNVLSGNWYFDEETFEAVSDEAKDFVSNLIVKDQARMNAAQCLAHPWLNNLAE
KAKRCNRRLKSQILFKKYLMKRRWKKNFIAVSAANRFKKISSSGALMALGV

| | | |
|---|---|---|
| NOV18p, SNP13375543 of | SEQ ID NO: 241 | 1788 bp |
| CG51448-01, DNA Sequence | ORF Start: ATG at 1 | ORF Stop: TGA at 1786 |
| | SNP Pos: 1724 | SNP Change: T to G |

ATGGCGACAGAAAATGGAGCAGTTGAGCTGGGAATTCAGAACCCATCAACAGACAAGGCACCTAAAGG
TCCCACAGGTGAAAGACCCCTGGCTGCAGGGAAAGACCCTGGCCCCCCAGACCCAAAGAAAGCTCCGG
ATCCACCCACCCTGAAGAAAGATGCCAAAGCCCCTGCCTCAGAGAAAGGGGATGGTACCCTGGCCCAA
CCCTCAACTAGCAGCCAAGGCCCCAAAGGAGAGGGTGACAGGGGCGGGGGGCCCGCGGAGGGCAGTGC
TGGGCCCCCGGCAGCCCTGCCCCAGCAGACTGCGACACCTGAGACCAGCGTCAAGAAGCCCAAGGCTG
AGCAGGGAGCCTCAGGCAGCCAGGATCCTGGAAAGCCCAGGGTGGGCAAGAAGGCAGCAGAGGGCCAA
GCAGCAGCCAGGAGGGGCTCACCTGCCTTTCTGCATAGCCCCAGCTGTCCTGCCATCATCTCCAGTTC
TGAGAAGCTGCTGGCCAAGAAGCCCCCAAGCGAGGCATCAGAGCTGACCTTTGAAGGGGTGCCCATGA
CCCACAGCCCCACGGATCCCAGGCCAGCCAAGGCAGAAGAAGGAAAGAACATCCTGGCAGAGAGCCAG
AAGGAAGTGGGAGAGAAAACCCCAGGCCAGGCTGGCCAGGCTAAGATGCAAGGGGACACCTCGAGGGG
GATTGAGTTCCAGGCTGTTCCCTCAGAGAAATCCGAGGTGGGGCAGGCCCTCTGTCTCACAGCCAGGG
AGGAGGACTGCTTCCAGATTTTGGATGATTGCCCGCCACCTCCGGCCCCCTTCCCTCACCGCATGGTG
GAGCTGAGGACCGGGAATGTCAGCAGTGAATTCAGTATGAACTCCAAGGAGGCGCTCGGAGGGGGCAA
GTTTGGGGCAGTCTGTACCTGCATGGAGAAAGCCACAGGCCTCAAGCTGGCAGCCAAGGTCATCAAGA
AACAGACTCCCAAAGACAAGGAAATGGTGTTGCTGGAGATTGAGGTCATGAACCAGCTGAACCACCGC
AATCTGATCCAGCTGTATGCAGCCATCGAGACTCCGCATGAGATCGTCCTGTTCATGGAGATCGAGGG
CGGAGAGCTCTTCGAGAGGATTGTGGATGAGGACTACCATCTGACCGAGGTGGACACCATGGTGTTTG
TCAGGCAGATCTGTGACGGGATCCTCTTCATGCACAAGATGAGGGTTTTGCACCTGGACCTCAAGCCA
GAGAACATCCTGTGTGTCAACACCACCGGGCATTTGGTGAAGATCATTGACTTTGGCCTGGCACGGAG
GTACCACAACCCCAACGAGAAGCTGAAGGTGAACTTTGGGACCCCAGAGTTCCTGTCACCTGAGGTGG
TGAATTATGACCAAATCTCCGATAAGACAGACATGTGGAGTATGGGGGTGATCACCTACATGCTGCTG
AGCGGCCTCTCCCCCTTCCTGGGAGATGATGACACAGAGACCCTAAACAACGTTCTATCTGGCAACTG
GTACTTTGATGAAGAGACCTTTGAGGCCGTATCAGACGAGGCCAAAGACTTTGTCTCCAACCTCATCG
TCAAGGACCAGGCCCGGATGAACGCTGCCCAGTGTCTCGCCCATCCCTGGCTCAACAACCTGGCGGAG
AAAGCCAAACGCTGTAACCGACGCCTTAAGTCCCAGATCTTGCTTAAGAAATACCTCATGAAGAGGCG
CTGGAAGAAAAACTTCATTGCTGGCAGCGCTGCCAACCGCTTCAAGAAGATCAGCAGCTCGGGGGCAC
TGATGGCTCTGGGGGTCTGA

| | | | |
|---|---|---|---|
| NOV18p, SNP13375543 of | SEQ ID NO: 242 | 595 aa | MW at 64459.8kD |
| CG51448-01, Protein | SNP Pos: 575 | | SNP Change: Val to Gly |
| Sequence | | | |

MATENGAVELGIQNPSTDKAPKGPTGERPLAAGKDPGPPDPKKAPDPPTLKKDAKAPASEKGDGTLAQ
PSTSSQGPKGEGDRGGGPAEGSAGPPAALPQQTATPETSVKKPKAEQGASGSQDPGKPRVGKKAAEGQ
AAARRGSPAFLHSPSCPAIISSSEKLLAKKPPSEASELTFEGVPMTHSPTDPRPAKAEEGKNILAESQ
KEVGEKTPGQAGQAKMQGDTSRGIEFQAVPSEKSEVGQALCLTAREEDCFQILDDCPPPPAPFPHRMV
ELRTGNVSSEFSNNSKEALGGGKFGAVCTCMEKATGLKLAAKVIKKQTPKDKEMVLLEIEVMNQLNHR
NLIQLYAAIETPHEIVLFMEIEGGELFERIVDEDYHLTEVDTMVFVRQICDGILFMHKMRVLHLDLKP
ENILCVNTTGHLVKIIDFGLARRYHNPNEKLKVNFGTPEFLSPEVVNYDQISDKTDMWSMGVITYMLL
SGLSPFLGDDDTETLMNVLSGNWYFDEETFEAVSDEAKDFVSNLIVKDQARMNAAQCLAHPWLNNLAE
KAKRCNRRLKSQILLKKYLMKRRWKKNFIAGSAANRFKKISSSGALMALGV

A ClustalW comparison of the above protein sequences yields the following sequence alignment shown in Table 18B.

TABLE 18B
Comparison of the NOV18 protein sequences.

NOV18a ------------------------------------------------------------
NOV18b -----MATENGAVELGIQNPSTDKAPKGPTGERPLAAGKDPGPPDPKKAPDPPTLKKDAK
NOV18c ------------------------------------------------------------
NOV18d TKLPTMATENGAVELGIQNPSTDKAPKGPTGERPLAAGKDPGPPDPKKAPDPPTLKKDAK
NOV18e -----MATENGAVELGIQNPSTDKAPKGPTGERPLAAGKDPGPPDPKKAPDPPTLKKDAK
NOV18f -----MATENGAVELGIQNPSTDKAPKGPTGERPLAAGKDPGPPDPKKAPDPPTLKKDAK
NOV18g -----MATENGAVELGIQNPSTDKAPKGPTGERPLAAGKDPGPPDPKKAPDPPTLKKDAK
NOV18a -------------------MPKPLPQRKG-MVPWPNPQLAALPQQTATPETSVKKPKAEQ
NOV18b APASEKGDGTLAQPSTSSQGPKGEGDRGGGPAEGSAGPPAALPQQTATPETSVKKPKAEQ
NOV18c ------------------------------------------------------------
NOV18d APASEKGDGTLAQPSTSSQGPKGEGDRGGGPAEGSAGPPAALPQQTATPETSVKKPKAEQ
NOV18e APASEKGDGThAQPSTSSQGPKGEGDRGGGPAEGSAGPPAALPQQTATPETSVKKPKAEQ
NOV18f APASEKGDGTLAQPSTSSQGPKGEGDRGGGPAEGSAGPPAALPQQTATPETSVKKPKAEQ
NOV18g APASEKGDGTLAQPSTSSQGPKGEGDRGGGPAEGSAGPPAALPQQTATPETSVKKPKAEQ
NOV18a GASGSQDPGKPRVGKKAAEGQAAARRGSPAFLHSPSCPAIISSSEKLLAKKPPSEASELT
NOV18b GASGSQDPGKPRVGKKAAEGQAAARRGSPAFLHSPSCPAIISSSEKLLAKKPPSEASELT
NOV18c ------------------------------------------------------------
NOV18d GASGSQDPGKPRVGKKAAEGQAAARRGSPAFLHSPSCPAIISSSEKLLAKKPPSEASELT
NOV18e GASGSQDPGKPRVGKKAAEGQAAARRGSPAFLHSPSCPAIISSSEKLLAKKPPSEASELT
NOV18f GASGSQDPGKPRVGKKAAEGQAAARRGSPAFLHSPSCPAIISSSEKLLAKKPPSEASELT
NOV18g GASGSQDPGKPRVGKKAAEGQAAARRGSPAFLHSPSCPAIISSSEKLLAKKPPSEASELT
NOV18a FEGVPMTHSPTDPRPAKAEEGKNILAESQKEVGEKTPGQAGQAKMQGDTSRGIEFQAVPS
NOV18b FEGVPMTHSPTDPRPAKAEEGKNILAESQKEVGEKTPGQAGQAXMQGDTSRGIEFQAVPS
NOV18c ------------------------------------------------------------
NOV18d FEGVPMTHSPTDPRPAKAEEGKNILAESQKEVGEKTPGQAGQAKMQGDTSRGIEFQAVPS
NOV18e FEGVPMTHSPTDPRPAKAEEGKNThAESQKEVGEKTPGQAGQAKMQGDTSRGIEFQAVPS
NOV18f FEGVPMTHSPTDPRPAKAEEGKNILAESQKEVGEKTPGQAGQAKMQGDTSRGIEFQAVPS
NOV18g FEGVPMTHSPTDPRSAKAEEGKNILAESQKEVGEKTPGQAGQAKMQGDTSRGIEFQAVPS
NOV18a EKSEVGQALCLTAREEDCFQILDDCPPPPAPFPHRMVELRTGNVSSEFSMNSKEALGGGK
NOV18b EKSEVGQALCLTAREEDCFQILDDCPPPPAPFPHRMVELRTGNVSSEFSMNSKEALGGGK
NOV18c ----------------------------------------------TKLMNSKEALGGGK
NOV18d EKSEVGQALCLTAREEDCFQILDDCPPPPAPFPHRMVELRTGNVSSEFSMNSKEALGGGK
NOV18e EKSEVGQALCLTAREEDCFQILDDCPPPPAPFPHRMVELRTGNVSSEFSMNSKEALGGGK
NOV18f EKSEVGQALCLTAREEDCFQILDDCPPPPAPFPHRMVELRTGNVSSEFSMNSKEALGGGK
NOV18g EKSEVGQALCLTAREEDCFQILDDCPPPPAPFPHRMVELRTGNVSSEFSMNSKEALGGGK
NOV18a FGAVCTCMEKATGLKLAAKVIKKQTPKDKEMVLLEIEVMNQLNHRNLIQLYAPJETPHEI
NOV18b FGAVCTCMEKATGLKIAAKVIKKQTPKDKEMVLLEIEVMNQLNHRNLIQLYAAIETPHEI
NOV18c FGAVCTCMEKATGLKLAAKVIKKQTPKDKEMVLLEIEVMNQLNHRNLIQLYAAIETPHEI
NOV18d FGAVCTCMEKATGLKLAAKVIKKQTPKDKEMVLLEIEVMNQLNHRNLIQLYAAIETPHEI
NOV18e FGAVCTCMEKATGLKLAAKVIKKQTPKDKEMVLLEIEVMNQLNHRNLIQLYAAIETPHEI
NOV18f FGAVCTCMEKATGLKLAAKVIKKQTPKDKEMVLLEIEVMNQLNHRNLIQLYAAIETPHEI
NOV18g FGAVCTCMEKATGLKLAAKVIKKQTPKDKEMVLLEIEVMNQLNHRNLIQLYAAIETPHEI
NOV18a VLFMEYIEGGELFERIVDEDYHLTEVDTMVFVRQICDGILFMHKMRVLHLDLKPENILCV
NOV18b VLFMEIE-GGELFERIVDEDYHLTEVDTMVFVRQICDGILFMHKMRVLHLDLKPENILCV
NOV18c VLFMEYIEGGELFERIVDEDYHLTEVDTMVFVRQICDGILFMHKMRVLHLDLKPENILCV
NOV18d VLFMEYIEGGELFERIVDEDYHLTEVDTMVFVRQICDGILFMHKMRVLHLDLKPENILCV
NOV18e VLFMEYIEGGELFERIVDEDYHLTEVDTMVFVRQICDGILFMHKMRVLHLDLKPENILCV
NOV18f VLFMEYIEGGELFERIVDEDYHLTEVDTMVFVRQICDGILFMHKMRVLHLDLKPENILCV
NOV18g VLFMEIE-GGELFERIVDEDYHLTEVDTMVFVRQICDGILLMHKMRVLHLDLKPENILCV
NOV18a NTTGHLVKIIDFGLARRYN-PNEKLKVNFGTPEFLSPEVVNYDQISDKTDMWSMGVITYM
NOV18b NTTGHLVKIIDFGLARRYHNPNEKLKVNFGTPEFLSPEVVNYDQISDKTDMWSMGVITYM
NOV18c NTTGHLVKIIDFGLARRYN-PNEKLKVNFGTPEFLSPEVVNYDQISDKTDMWSMGVITYM
NOV18d NTTGHLVKIIDFGLARRYN-PNEKLKVNFGTPEFLSPEVVNYDQISDKTDMWSMGVITYM
NOV18e NTTGHLVKIIDFGLARRYN-PNEKLKVNFGTPEFLSPEAVNYDQISDKTDMWSMGVITYM
NOV18f NTTGHLVKIIDFGLARRYN-PNEKLKVNFGTPEFLSPEAVNYDQISDKTDMWSMGVITYM
NOV18g NTTGHLVKIIDFGLARRYN-PNEKLKVNFGTPEFLSPEVVNYDQISDKTDMWSMGVITYM
NOV18a LLSGLSPFLGDDDTETLNNVLSGNWYFDEETFEAVSDEAKDFVSNLIVKDQRARNNAAQC
NOV18b LLSGLSPFLGDDDTETLNNVLSGNWYFDEETFEAVSDEAKDFVSNLIVKDQ-ARNNAAQC
NOV18c LLSGLSPFLGDDDTETLNNVLSGNWYFDEETFEAVSDEAKDFVSNLIVKDQRARMNAAQC
NOV18d LLSGLSPFLGDDDTETLNNVLSGNWYFDEETFEAVSDEAKDFVSNLIVKDQRARMNAAQC
NOV18e LLSGLSPFLGDDDTETLNNVLSGNWYFDEETFEAVSDEAKDFVSNLIVKDQRARNNAAQC
NOV18f LLSGLSPFLGDDDTETLNNVLSGNWYFDEETFEAVSDEAKDFVSNLIVKDQRARNNAAQC
NOV18g LLSGLSPFLGDDDTETLNNVLSGNWYFDEETFEAVSDEAKDFVSNLIVKDQRARMNAAQC
NOV18a LAHPWLNNLAEKAKRCNRRLKSQILLKKYLMKRRWKKNFIAVSAANRFKKISSSGALMAL
NOV18b LAHPWLNNLAEKAKRCNRRLKSQILLKKYLMKRRWKKNFIAVSAANRFKKISSSGALMAL
NOV18c LAHPWLVDG---------------------------------------------------
NOV18d LAHPWLNNLAEKAKRCNRRLKSQILLKKYLMKRRWKKNFIAVSAANRFKKISSSGALMAL
NOV18e LAHPWLNNLAEKAKRCNRRLKSQILLKKYLMKRRWKKNFIAVSAANRFKKISSSGALMAL
NOV18f LAHPWLNNLAEKAKRCNRRLKSQILLKKYLMKRRWKKNFIAVSAANRFKKISSSGALMAL
NOV18g LAHPWLNNLAEKAKRCNRRLKSQILLKKYLMKRRWKKNFIAVSAANRFKKISSSGALMAL
NOV18a GV---
NOV18b GV---
NOV18c -----
NOV18d GVVDG
NOV18e GV---
NOV18f GV---
NOV18g GV---
NOV18a (SEQ ID NO: 212)
NOV18b (SEQ ID NO: 214)
NOV18c (SEQ ID NO: 216)
NOV18d (SEQ ID NO: 218)
NOV18e (SEQ ID NO: 220)
NOV18f (SEQ ID NO: 222)
NOV18g (SEQ ID NO: 224)

Further analysis of the NOV18a protein yielded the following properties shown in Table 18C.

TABLE 18C
Protein Sequence Properties NOV18a

SignalP analysis: No Known Signal Sequence Indicated
PSORT II analysis:

| PSG: | a new signal peptide prediction method |
|---|---|
| | N-region: length 9; pos.chg 3; neg.chg 0 |
| | H-region: length 20; peak value 1.84 |
| | PSG score: −2.56 |
| GvH: | von Heijne's method for signal seq. recognition |
| | GvH score (threshold: −2.1): −10.69 |
| | possible cleavage site: between 29 and 30 |

>>> Seems to have no N-terminal signal peptide

| ALOM: | Klein et al's method for TM region allocation |
|---|---|
| | Init position for calculation: 1 |
| | Tentative number of TMS(s) for the threshold 0.5: 0 |
| | number of TMS(s) . . . fixed |
| | PERIPHERAL Likelihood = 1.48 (at 393) |
| | ALOM score: 1.48 (number of TMSs: 0) |

MITDISC: discrimination of mitochondrial targeting seq

| | | | |
|---|---|---|---|
| R content: | 1 | Hyd Moment(75): | 14.68 |
| Hyd Moment(95): | 9.57 | G content: | 1 |
| D/E content: | 1 | S/T content: | 2 |
| Score: —2.37 | | | |

Gavel: prediction of cleavage sites for mitochondrial preseq

R-2 motif at 18 QRK|GM

NUCDISC: discrimination of nuclear localization signals

pat4: KKPK (4) at 34
pat7: none
bipartite: RRLKSQILLKKYLMKRR at 477
content of basic residues: 12.9%
NLS Score: 0.27

KDEL: ER retention motif in the C-terminus: none
ER Membrane Retention Signals: none
SKL: peroxisomal targeting signal in the C-terminus: none
PTS2: 2nd peroxisomal targeting signal: found

RIVDEDYHL at 295

VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 55.5

COIL: Lupas's algorithm to detect coiled-coil regions

total: 0 residues

--------------------------
Final Results (k = 9/23):

69.6%: nuclear
17.4%: mitochondrial
13.0%: cytoplasmic

>> prediction for CG51448-05 is nuc (k = 23)

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE22849 | Human cardiac myosin light | 20 . . . 521 | 502/502 (100%) | 0.0 |
| | chain kinase (cMLCK) mutant, | 95 . . . 596 | 502/502 (100%) | |
| | G89D - Homo sapiens, 596 aa. | | | |
| | [WO200224889-A2, 28-MAR- | | | |
| | 2002] | | | |
| AAE22847 | Human cardiac myosin light | 20 . . . 521 | 502/502 (100%) | 0.0 |
| | chain kinase (cMLCK) mutant, | 95 . . . 596 | 502/502 (100%) | |
| | A87V - Homo sapiens, 596 aa. | | | |
| | [WO200224889-A2, 28-MAR- | | | |
| | 2002] | | | |
| AAE22723 | Human cardiac myosin light | 20 . . . 521 | 502/502 (100%) | 0.0 |
| | chain kinase (cMLCK) protein - | 95 . . . 596 | 502/502 (100%) | |
| | Homo sapiens, 596 aa. | | | |
| | [WO200224889-A2, 28-MAR- | | | |
| | 2002] | | | |
| AAU03521 | Human protein kinase #21 - | 20 . . . 521 | 502/502 (100%) | 0.0 |
| | Homo sapiens, 612 aa. | 111 . . . 612 | 502/502 (100%) | |
| | [WO200138503-A2, 31-MAY- | | | |
| | 2001] | | | |
| AAE16340 | Human POLY4 protein - Homo | 20 . . . 521 | 501/502 (99%) | 0.0 |
| | sapiens, 596 aa. [WO200185767- | 95 . . . 596 | 501/502 (99%) | |
| | A2, 15-NOV-2001] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC88318 | Sequence 7 from Patent | 20 . . . 521 | 502/502 (100%) | 0.0 |
| | WO0164905 - Homo sapiens | 95 . . . 596 | 502/502 (100%) | |
| | (Human), 596 aa. | | | |
| Q9H1R3 | Myosin light chain kinase 2, | 20 . . . 521 | 502/502 (100%) | 0.0 |
| | skeletal/cardiac muscle (EC | 94 . . . 595 | 502/502 (100%) | |
| | 2.7.1.117) (MLCK2) - Homo | | | |
| | sapiens (Human), 595 aa. | | | |
| A35021 | myosin-light-chain kinase (EC | 20 . . . 521 | 462/504 (91%) | 0.0 |
| | 2.7.1.117), skeletal muscle - | 105 . . . 608 | 479/504 (94%) | |
| | rabbit, 608 aa. | | | |
| P07313 | Myosin light chain kinase 2, | 20 . . . 521 | 462/504 (91%) | 0.0 |
| | skeletal/cardiac muscle (EC | 104 . . . 607 | 479/504 (94%) | |
| | 2.7.1.117) (MLCK2) - | | | |
| | Oryctolagus cuniculus (Rabbit), | | | |
| | 607 aa. | | | |
| A28798 | myosin-light-chain kinase (EC | 20 . . . 521 | 429/502 (85%) | 0.0 |
| | 2.7.1.117), skeletal muscle - rat, | 112 . . . 610 | 453/502 (89%) | |
| | 610 aa. | | | |

PFam analysis inidcates that the NOV18a protein contains the domains shown in the Table 18F.

TABLE 18F
Domain Analysis of NOV18a

| | | Identities/ | |
|---|---|---|---|
| | | Similarities | |
| | NOV18a Match | for the | |
| Pfam Domain | Region | Matched Region | Expect Value |
| pkinase | 210 . . . 465 | 93/297 (31%) | 9.3e-76 |
| | | 198/297 (67%) | |

### Example 19

The NOV19 clone was analyzed, and the nucleotide and encoded polypeptide sequences are shown in Table 19A.

TABLE 19A
NOV19 Sequence Analysis

| | | |
|---|---|---|
| NOV19a, CG51752-02 | SEQ ID NO: 243 | 1042 bp |
| DNA Sequence | ORF Start: ATG at 207 | ORF Stop: TGA at 1008 |

AGAGTGCTCTAAACCCAGCTCGGCCTTTGCTGTATTAGACAGAAGCACCTCATTCATATCCCTGGGGC
CCCTGATGGTGCAGTGGTCTGGCTGTGGTCTGCACACCAGCTATTCTGTTTTGTTTTGTTTTGTTTTT
TCCTACCTTTTTCCAATCCTCACACCTTCTGATCAACAGCCCCAGTAGGGTTTAAAGGTCCTAGAGCT
ACATGGGATTTAGGTTTCTGGGCACAGCCAATTCTGCCACTTTTGAGACTTCCCTTCCCCTTCCACTT
GCCCCTCTCTGGTTCTCTGCCACCAGTCCAGAAGAACTGAGTGTCGTGCTGGGGACCAACGACTTAAC
TAGCCCATCCATGGAAATAAAGGAGGTCGCCAGCATCATTCTTCACAAAGACTTTAAGAGAGCCAACA
TGGACAATGACATTGCCTTGCTGCTGCTGGCTTCGCCCATCAAGCTCGATGACCTGAAGGTGCCCATC
TGCCTCCCCACGCAGCCCGGCCCTGCCACATGGCGCGAATGCTGGGTGGCAGGTTGGGGCCAGACCAA
TGCTGCTGACAAAAACTCTGTGAAAACGGATCTGATGAAAGTGCCAATGGTCATCATGGACTGGGAGG
AGTGTTCAAAGATGTTTCCAAAACTTACCAAAAATATGCTGTGTGCCGGATACAAGAATGAGAGCTAT
GATGCCTGCAAGGGTGACAGTGGGGGGCCTCTGGTCTGCACCCCAGAGCCTGGTGAGAAGTGGTACCA
GGTGGGCATCATCAGCTGGGGAAAGAGCTGTGGAGAGAAGAACACCCCAGGGATATACACCTCGTTGG
TGAACTACAACCTCTGGATCGAGAAAGTGACCCAGCTAGAGGGCAGGCCCTTCAATGCAGAGAAAAGG
AGGACTTCTGTCAAACAGAAACCTATGGGCTCCCCAGTCTCGGGAGTCCCAGAGCCAGGCAGCCCCAG
ATCCTGGCTCCTGCTCTGTCCCCTGTCCCATGTGTTGTTCAGAGCTATTTTGTACTGATAATAAAATA
GAGGCTATTCTTTCAACCGAAA

| | | | |
|---|---|---|---|
| NOV19a, CG51752-02 | SEQ ID NO: 244 | 267 aa | MW at 29498.9kD |
| Protein Sequence | | | |

MGFRFLGTANSATFETSLPLPLAPLWFSATSPEELSVVLGTNDLTSPSMEIKEVASIILHKDFKRANM
DNDIALLLLASPIKLDDLKVPICLPTQPGPATWRECWVAGWGQTNAADKNSVKTDLMKVPMVIMDWEE
CSKMFPKLTKNNLCAGYKNESYDACKGDSGGPLVCTPEPGEKWYQVGIISWGKSCGEKNTPGIYTSLV
NYNLWIEKVTQLEGRPFNAEKRRTSVKQKPMGSPVSGVPEPGSPRSWLLLCPLSHVLFRAILY

| | | |
|---|---|---|
| NOV19b, CG51752-03 | SEQ ID NO: 245 | 888 bp |
| DNA Sequence | ORF Start: ATG at 79 | ORF Stop: TGA at 880 |

TTGTTTTTTCCTACCTTTTTCCAATCCTCACACCTTCTGATCAACAGCCCCAGTAGGGTTTAAAGGTC
CTAGAGCTACATGGGATTTAGGTTTCTGGGCACAGCCAATTCTGCCACTTTTGAGACTTCCCTTCCCC
TTCCACTTGCCCCTCTCTGGTTCTCTGCCACCAGTCCAGAAGAACTGAGTGTCGTGCTGGGGACCAAC
GACTTAACTAGCCCATCCATGGAAATAAAGGAGGTCGCCAGCATCATTCTTCACAAAGACTTTAAGAG
AGCCAACATGGACAATGACATTGCCTTGCTGCTGCTGGCTTCGCCCATCAAGCTCGATGACCTGAAGG
TGCCCATCTGCCTCCCCACGCAGCCCGGCCCTGCCACATGGCGCGAATGCTGGGTGGCAGGTTGGGGC
CAGACCAATGCTGCTGACAAAAACTCTGTGAAAACGGATCTGATGAAAGCGCCAATGGTCATCATGGA
CTGGGAGGAGTGTTCAAAGATGTTTCCAAAACTTACCAAAAATATGCTGTGTGCCGGATACAAGAATG
AGAGCTATGATGCCTGCAAGGGTGACAGTGGGGGGCCTCTGGTCTGCACCCCAGAGCCTGGTGAGAAG
TGGTACCAGGTGGGCATCATAACCTGGGGAAAGAGCTGTGGAGAGAAGAACACCCCAGGGATATACAC
CTCGTTGGTGAACTACAACCTCTGGATCGAGAAAGTGACCCAGCTAGAGGGCAGGCCCTTCAATGCAG
AGAAAAGGAGGACTTCTGTCAAACAGAAACCTATGGGCTCCCCAGTCTCGGGAGTCCCAGAGCCAGGC
AGCCCCAGATCCTGGCTCCCGCTCTGTCCCCTGTCCCATGTGTTGTTCAGAGCTATTTTGTACTGATT
ATAA

| | | | |
|---|---|---|---|
| NOV19b, CG51752-03 | SEQ ID NO: 246 | 267 aa | MW at 29468.8kD |
| Protein Sequence | | | |

MGFRFLGTANSATFETSLPLPLAPLWFSATSPEELSVVLGTNDLTSPSMEIKEVASIILHKDFKRANM
DNDIALLLLASPIKLDDLKVPICLPTQPGPATWRECWVAGWGQThAADKNSVKTDLMKAPMVIMDWEE
CSKMFPKLTKNMLCAGYKNESYDACKGDSGGPLVCTPEPGEKWYQVGIITWGKSCGEKNTPGIYTSLV
NYNLWIEKVTQLEGRPFNAEKRRTSVKQKPMGSPVSGVPEPGSPRSWLPLCPLSHVLFRAILY

| | | |
|---|---|---|
| NOV19c, 175069825 | SEQ ID NO:247 | 816 bp |
| DNA Sequence | ORF Start: at 1 | ORF Stop: end of sequence |

GGATCCACCATGGGATTTAGGTTTCTGGGCACAGCCAATTCTGCCGCTTTTGAGACTTCCCTTCCCCT
TCCACTTGCCCCTCTCTGGTTCTCTGCCACCAGTCCAGAAGAACTGAGTGTCGTGCTGGGGACCAACG
ACTTAACTAGCCCATCCATGGAAATAAAGGAGGTCGCCAGCATCATTCTTCACAAAGACTTTAAGAGA
GCCAACATGGACAATGACATTGCCTTGCTGCTGCTGGCTTCGCCCATCAAGCTCGATGACCTGAAGGT
GCCCATCTGCCTCCCCACGCAGCCCGGCCCTGCCACATGGCGCGAATGCTGGGTGGCAGGTTGGGGCC
AGACCAATGCTGCTGACAAAAACTCTGTGAAAACGGATCTGATGAAAGCGCCAATGGTCATCATGGAC
TGGGAGGAGTGTTCAAAGATGTTTCCAAAACTTACCAAAAATATGCTGTGTGCCGGATACAAGAATGA
GAGCTATGATGCCTGCAAGGGTGACAGTGGGGGGCCTCTGGTCTGCACCCCAGAGCCTGGTGAGAAGT
GGTACCAGGTGGGCATCATAAGCTGGGGAAAGAGCTGTGGAGAGAAGAACACCCCAGGGATATACACC
TCGTTGGTGAACTACAACCTCTGGATCGAGAAAGTGACCCAGCTAGAGGGCAGGCCCTTCAATGCAGA
GAAAAGGAGGACTTCTGTCAAACAGAAACCTATGGGCTCCCCAGTCTCGGGAGTCCCAGAGCCAGGCA
GCCCCAGATCCTGGCTCCTGCTCTGTCCCCTGTCCCATGTGTTGTTCAGAGCTATTTTGTACCTCGAG

| | | | |
|---|---|---|---|
| NOV19c, 175069825 | SEQ ID NO: 248 | 272 aa | MW at 29928.3kD |
| Protein Sequence | | | |

GSTMGFRFLGTANSAAFETSLPLPLAPLWFSATSPEELSVVLGTNDLTSPSMEIKEVASIILHKDFKR
ANMDNDIALLLLASPIKLDDLKVPICLPTQPGPATWRECWVAGWGQTNAADKNSVKTDLMKAPMVIMD
WEECSKMFPKLTKNMLCAGYKNESYDACKGDSGGPLVCTPEPGEKWYQVGIISWGKSCGEKNTPGIYT
SLVNYNLWIEKVTQLEGRPFNAEKRRTSVKQKPMGSPVSGVPEPGSPRSWLLLCPLSHVLFRAILYLE

| | | |
|---|---|---|
| NOV19d, 175069842 | SEQ ID NO: 249 | 816 bp |
| DNA Sequence | ORF Start: at 1 | ORF Stop: end of sequence |

GGATCCACCATGGGATTTAGGTTTCTGGGCACAGCCAATTCTGCCGCTTTTGAGACTTCCCTTCCCCT
TCCACTTGCCCCTCTCTGGTTCTCTGCCACCAGTCCAGAAGAACTGAGTGTCGTGCTGGGGACCAACG
ACTTAACTAGCCCATCCATGGAAATAAAGGAGGTCGCCAGCATCATTCTTCACAAAGACTTTAAGAGA
GCCAACATGGACAATGACATTGCCTTGCTGCTGCTGGCTTCGCCCATCAAGCTCGATGACCTGAAGGT
GCCCATCTGCCTCCCCACGCAGCCCGGCCCTGCCACATGGCGCGAATGCTGGGTGGCAGGTTGGGGCC
AGACCAATGCTGCTGACAAAAACTCTGTGAAAACGGATCTGATGAAAGCGCCAATGGTCATCATGGAC
TGGGAGGAGTGTTCAAAGATGTTTCCAAAACTTACCAAAAATATGCTGCGTGCCGGATACAAGAATGA
GAGCTATGATGCCTGCAAGGGTGACAGTGGGGGGCCTCTGGTCTGCACCCCAGAGCCTGGTGAGAAGT
GGTACCAGGTGGGCATCATAAGCTGGGGAAAGAGCTGTGGAGAGAAGAACACCCCAGGGATATACACC
TCGTTGGTGAACTACAACCTCTGGATCGAGAAAGTGACCCAGCTAGAGGGCAGGCCCTTCAATGCAGA
GAAAAGGAGGACTTCTGTCAAACAGAAACCTATGGGCTCCCCAGTCTCGGGAGTCCCAGAGCCAGGCA
GCCCCAGATCCTGGCTCCTGCTCTGTCCCCTGTCCCATGTGTTGTTCAGAGCTATTTTGTACCTCGAG

| | | | |
|---|---|---|---|
| NOV19d, 175069842 | SEQ ID NO: 250 | 272 aa | MW at 29981.3kD |
| Protein Sequence | | | |

GSTMGFRFLGTANSAAFETSLPLPLAPLWFSATSPEELSVVLGTNDLTSPSMEIKEVASIILHKDFKR
ANMDNDIALLLLASPIKLDDLKVPICLPTQPGPATWRECWVAGWGQTNAADKNSVKTDLMKAPMVIMD
WEECSKMFPKLTKNMLRAGYKNESYDACKGDSGGPLVCTPEPGEKWYQVGIISWGKSCGEKNTPGIYT
SLVNYNLWIEKVTQLEGRPFNAEKRRTSVKQKPMGSPVSGVPEPGSPRSWLLLCPLSHVLFRAILYLE

| | | |
|---|---|---|
| NOV19e, 258076315 | SEQ ID NO: 251 | 729 bp |
| DNA Sequence | ORF Start: at 1 | ORF Stop: end of sequence |

GGATCCGCCACCAGTCCAGAAGAACTGAGTGTCGTGCTGGGGACCAACGACTTAACTAGCCCATCCAT
GGAAATAAAGGAGGTCGCCAGCATCATTCTTCACAAAGACTTTAAGAGAGCCAACATGGACAATGACA
TTGCCTTGCTGCTGCTGGCTTCGCCCATCAAGCTCGATGACCTGAAGGTGCCCATCTGCCTCCCCACG
CAGCCCGGCCCTGCCACATGGCGCGAATGCTGGGTGGCAGGTTGGGGCCAGACCAATGCTGCTGACAA
AAACTCTGTGAAAACGGATCTGATGAAAGCGCCAATGGTCATCATGGACTGGGAGGAGTGTTCAAAGA
TGTTTCCAAAACTTACCAAAAATATGCTGTGTGCCGGATACAAGAATGAGAGCTATGATGCCTGCAAG
GGTGACAGTGGGGGGCCTCTGGTCTGCACCCCAGAGCCTGGTGAGAAGTGGTACCAGGTGGGCATCAT
CAGCTGGGGAAAGAGCTGTGGAGAGAAGAACACCCCAGGGATATACACCTCGTTGGTGAACTACAACC
TCTGGATCGAGAAAGTGACCCAGCTAGAGGGCAGGCCCTTCAATGCAGAGAAAAGGAGGACTTCTGTC
AAACAGAAACCTATGGGCTCCCCAGTCTCGGGAGTCCCAGAGCCAGGCAGCCCCAGATCCTGGCTCCT
GCTCTGTCCCCTGTCCCATGTGTTGTTCAGAGCTATTTTGTACCTCGAG

| | | | |
|---|---|---|---|
| NOV19e, 258076315 | SEQ ID NO: 252 | 243 aa | MW at 26802.7kD |
| Protein Sequence | | | |

GSATSPEELSVVLGTNDLTSPSMEIKEVASIILHKDFKRANMDNDIALLLLASPIKLDDLKVPICLPT
QPGPATWRECWVAGWGQTNAADKNSVKTDLMKAPMVIMDWEECSKMFPKLTKNMLCAGYKNESYDACK
GDSGGPLVCTPEPGEKWYQVGIISWGKSCGEKNTPGIYTSLVNYNLWIEKVTQLEGRPFNAEKRRTSV
KQKPMGSPVSGVPEPGSPRSWLLLCPLSHVLFRAILYLE

| | | |
|---|---|---|
| NOV19f, 258076366 | SEQ ID NO: 253 | 729 bp |
| DNA Sequence | ORF Start: at 1 | ORF Stop: end of sequence |

GGATCCGCCACCAGTCCAGAAGAACTGAGTGTCGTGCTGGGGACCAACGACTTAACTAGCCCATCCAT
GGAAATAAAGGAGGTCGCCAGCATCATTCTTCACAAAGACTTTAAGAGAGCCAACATGGACAATGACA
TTGCCTTGCTGCTGCTGGCTTCGCCCATCAAGCTCGATGACCTGAAGGTGCCCATCTGCCTCCCCACG
CAGCCCGGCCCTGCCACATGGCGCGAATGCTGGGTGGCAGGTTGGGGCCAGACCAATGCTGCTGACAA
AAACTCTGTGAAAACGGATCTGATGAAAGTGCCAATGGTCATCATGGACTGGGAGGAGTGTTCAAAGA
TGTTTCCAAAACTTACCAAAAATATGCTGTGTGCCGGATACAAGAATGAGAGCTATGATGCCTGCAAG
GGTGACAGTGGGGGGCCTCTGGTCTGCACCCCAGAGCCTGGTGAGAAGTGGTACCAGGTGGGCATCAT
CAGCTGGGGAAAGAGCTGTGGAGAGAAGAACACCCCAGGGATATACACCTCGTTGGTGAACTACAACC
TCTGGATCGAGAAAGTGACCCAGCTAGAGGGCAGGCCCTTCAATGCAGAGAAAAGGAGGACTTCTGTC
AAACAGAAACCTATGGGCTCCCCAGTCTCGGGAGTCCCAGAGCCAGGCAGCCCCAGATCCTGGCTCCT
GCTCTGTCCCCTGTCCCATGTGTTGTTCAGAGCTATTTTGTACCTCGAG

| | | | |
|---|---|---|---|
| NOV19f, 258076366 | SEQ ID NO: 254 | 243 aa | MW at 26830.8kD |
| Protein Sequence | | | |

GSATSPEELSVVLGTNDLTSPSMEIKEVASIILHKDFKRANMDNDIALLLLASPIKLDDLKVPICLPT
QPGPATWRECWVAGWGQTNAADKNSVKTDLMKVPMVIMDWEECSKMFPKLTKNMLCAGYKNESYDACK
GDSGGPLVCTPEPGEKWYQVGIISWGKSCGEKNTPGIYTSLVNYNLWIEKVTQLEGRPFNAEKRRTSV
KQKPMGSPVSGVPEPGSPRSWLLLCPLSHVLFRAILYLE

| | | |
|---|---|---|
| NOV19g, CG51752-04 | SEQ ID NO: 255 | 852 bp |
| DNA Sequence | ORF Start: ATG at 46 | ORF Stop: TGA at 847 |

CTTCTGATCAACAGCCCCAGTAGGGTTTAAAGGTCCTAGAGCTACATGGGATTTAGGTTTCTGGGCAC
AGCCAATTCTGCCACTTTTGAGGCTTCCCTTCCCCTTCCACTTGCCCCTCTCTGGTTCTCTGCCCCCA
GTCCAGAAGAACTGAGTGTCGTGCTGGGGACCAACGACTTAACTAGCCCATCCATGGAAATAAAGGAG
GTCGCCAGCATCATTCTTCACAAAGACTTTAAGAGAGCCAACATGGACAATGACATTGCCTTGCTGCT
GCTGGCTTCGCCCATCAAGCTCGATGACCTGAAGGTGCCCATCTGCCTCCCCACGCAGCCCGGCCCTG
CCACATGGCGCGAATGCTGGGTGGCAGGTTGGGGCCAGACCAATGCTGCTGACAAAAACTCTGTGAAA
ACGGATCTGATGAAAGCGCCAATGGTCATCATGGACTGGGAGGAGTGTTCAAAGATGTTTCCAAAACT
TACCAAAAATATGCTGTGTGCCGGATACAAGAATGAGAGCTATGATGCCTGCAAGGGTGACAGTGGGG
GGCCTCTGGTCTGCACCCCAGAGCCTGGTGAGAAGTGGTACCAGGTGGGCATCATCAGCTGGGGAAAG
AGCTGTGGAGAGAAGAACACCCCAGGGATATACACCTCGTTGGTGAATTACAACCTCTGGATCGAGAA
AGTGACCCAGCTAGAGGGCAGGCCCTTCAATGCAGAGAAAAGGAGGACTTCTGTCAAACAGAAACCTA
TGGGCTCCCCAGTCTCGGGAGTCCCAGAGCCAGGCAGCCCCAGATCCTGGCTCCTGCTCTGTCCCCTG
TCCCATGTGTTGTTCAGAGCTATTTTGTACTGATAA

| | | | |
|---|---|---|---|
| NOV19g, CG51752-04 | SEQ ID NO: 256 | 267 aa | MW at 29436.8kD |
| Protein Sequence | | | |

MGFRFLGTANSATFEASLPLPLAPLWFSAPSPEELSVVLGTNDLTSPSMEIKEVASIILHKDFKRANN
DNDIALLLLASPIKLDDLKVPICLPTQPGPATWRECWVAGWGQTNAADKNSVKTDLMKAPMVIMDWEE
CSKMFPKLTKNMLCAGYKNESYDACKGDSGGPLVCTPEPGEKWYQVGIISWGKSCGEKNTPGIYTSLV
NYNLWIEKVTQLEGRPFNAEKRRTSVKQKPMGSPVSGVPEPGSPRSWLLLCPLSHVLFRAILY

| | | |
|---|---|---|
| NOV19h, 191887409 | SEQ ID NO: 257 | 816 bp |
| DNA Sequence | ORF Start: at 1 | ORF Stop: end of sequence |

GGATCCACCATGGGATTTAGGTTTCTGGGCACAGCCAATTCTGCCACTTTTGAGACTTCCCTTCCCCT
TCCACTTGCCCCTCTCTGGTTCTCTGCCACCAGTCCAGAAGAACTGAGTGTCGTGCTGGGGACCAACG
ACTTAACTAGCCCATCCATGGAAATAAAGGAGGTCGCCAGCATCATTCTTCACAAAGACTTTAAGAGA
GCCAACATGGACAATGACATTGCCTTGCTGCTGCTGGCTTCGCCCATCAAGCTCGATGACCTGAAGGT
GCCCATCTGCCTCCCCACGCAGCCCGGCCCTGCCACATGGCGCGAATGCTGGGTGGCAGGTTGGGGCC
AGACCAATGCTGCTGACAAAAACTCTGTGAAAACGGATCTGATGAAAGTGCCAATGGTCATCATGGAC
TGGGAGGAGTGTTCAAAGATGTTTCCAAAACTTACCAAAAATATGCTGTGTGCCGGATACAAGAATGA
GAGCTATGATGCCTGCAAGGGTGACAGTGGGGGGCCTCTGGTCTGCACCCCAGAGCCTGGTGAGAAGT
GGTACCAGGTGGGCATCATCAGCTGGGGAAAGAGCTGTGGAGAGAAGAACACCCCAGGGATATACACC
TCGTTGGTGAACTACAACCTCTGGATCGAGAAAGTGACCCAGCTAGAGGGCAGGCCCTTCAATGCAGA
GAAAAGGAGGACCTCTGTCAAACAGAAACCTATGGGCTCCCCAGTCTCGGGAGTCCCAGAGCCAGGCA
GCCCCAGATCCTGGCTCCTGCTCTGTCCCCTGTCCCATGTGTTGTTCAGAGCTATTTTGTACCTCGAG

| | | | |
|---|---|---|---|
| NOV19h, 191887409 | SEQ ID NO: 258 | 272 aa | MW at 29986.4kD |
| Protein Sequence | | | |

GSTMGFRFLGTANSATFETSLPLPLAPLWFSATSPEELSVVLGTNDLTSPSMEIKEVASIILHKDFKR
ANMDNDIALLLLASPIKLDDLKVPICLPTQPGPATWRECWVAGWGQTNAADKNSVKTDLMKVPMVIMD
WEECSKMFPKLTKNMLCAGYKNESYDACKGDSGGPLVCTPEPGEKWYQVGIISWGKSCGEKNTPGIYT
SLVNYLWIEKVTQLEGRPFNAEKRRTSVKQKPMGSPVSGVPEPGSPRSWLLLCPLSHVLFRAILYLE

| | | |
|---|---|---|
| NOV19i, CG51752-01 | SEQ ID NO: 259 | 1078 bp |
| DNA Sequence | ORF Start: ATG at 243 | ORF Stop: TGA at 1044 |

TTGATCCGTGCCAAGTGGCTTTTTGTGGGCTCTGTAGAGTGCTCTAAACCCAGCTCGGCCTTTGCTGT
ATTAGACAGAAGCACCTCATTCATATCCCTGGGGCCCCTGATGGTGCAGTGGTCTGGCTGTGGTCTGC
ACACCAGCTATTCTGTTTTGTTTTGTTTTGTTTTTTTCCTACCTTTTTCCAATCCTCACACCTTCTGA
TCAACAGCCCCAGTAGGGTTTAAAGGTCCTAGAGCTACATGGGATTTAGGTTTCTGGGCACAGCCAAT
TCTGCCACTTTTGAGACTTCCCTTCCCCTTCCACTTGCCCCTCTCTGGTTCTCTGCCACCAGTCCAGA
AGAACTGAGTGTCGTGCTGGGGACCAACGACTTAACTAGCCCATCCATGGAAATAAAGGAGGTCGCCA
GCATCATTCTTCACAAAGACTTTAAGAGAGCCAACATGGACAATGACATTGCCTTGCTGCTGCTGGCT
TCGCCCATCAAGCTCGATGACCTGAAGGTGCCCATCTGCCTCCCCACGCAGCCCGGCCCTGCCACATG
GCGCGAATGCTGGGTGGCAGGTTGGGGCCAGACCAATGCTGCTGACAAAAACTCTGTGAAAACGGATC
TGATGAAAGTGCCAATGGTCATCATGGACTGGGAGGAGTGTTCAAAGATGTTTCCAAAACTTACCAAA
AATATGCTGTGTGCCGGATACAAGAATGAGAGCTATGATGCCTGCAAGGGTGACAGTGGGGGGCCTCT
GGTCTGCACCCCAGAGCCTGGTGAGAAGTGGTACCAGGTGGGCATCATCAGCTGGGGAAAGAGCTGTG
GAGATAAGAACACCCCAGGGATATACACCTCGTTGGTGAACTACAACCTCTGGATCGAGAAAGTGACC
CAGCTAGGAGGCAGGCCCTTCAATGCAGAGAAAAGGAGGACTTCTGTCAAACAGAAACCTATGGGCTC
CCCAGTCTCGGGAGTCCCAGAGCCAGGCAGCCCCAGATCCTGGCTCCTGCTCTGTCCCCTGTCCCATG
TGTTGTTCAGAGCTATTTTGTACTGATAATAAAATAGAGGCTATTCTTTCAACCGAAA

| | | | |
|---|---|---|---|
| NOV19i, CG51752-01 | SEQ ID NO: 260 | 267 aa | MW at 29412.8kD |
| Protein Sequence | | | |

MGFRFLGTANSATFETSLPLPLAPLWFSATSPEELSVVLGTNDLTSPSMEIKEVASIILHKDFKRANN
DNDIALLLLASPIKLDDLKVPICLPTQPGPATWRECWVAGWGQTNAADKNSVKTDLMKVPMVIMDWEE
CSKMFPKLTKNMLCAGYKNESYDACKGDSGGPLVCTPEPGEKWYQVGIISWGKSCGDKNTPGIYTSLV
NYNLWIEKQLGGRPFNAEKRRTSVKQKPMGSPVSGVPEPGSPRSWLLLCPLSHVLFRAILY

| | | |
|---|---|---|
| NOV19j, CG51752-05 | SEQ ID NO: 261 | 816 bp |
| DNA Sequence | ORF Start: ATG at 10 | ORF Stop: at 811 |

GGATCCACCATGGGATTTAGGTTTCTGGGCACAGCCAATTCTGCCGCTTTTGAGACTTCCCTTCCCCT
TCCACTTGCCCCTCTCTGGTTCTCTGCCACCAGTCCAGAAGAACTGAGTGTCGTGCTGGGGACCAACG
ACTTAACTAGCCCATCCATGGAAATAAAGGAGGTCGCCAGCATCATTCTTCACAAAGACTTTAAGAGA
GCCAACATGGACAATGACATTGCCTTGCTGCTGCTGGCTTCGCCCATCAAGCTCGATGACCTGAAGGT
GCCCATCTGCCTCCCCACGCAGCCCGGCCCTGCCACATGGCGCGAATGCTGGGTGGCAGGTTGGGGCC
AGACCAATGCTGCTGACAAAAACTCTGTGAAAACGGATCTGATGAAAGCGCCAATGGTCATCATGGAC
TGGGAGGAGTGTTCAAAGATGTTTCCAAAACTTACCAAAAATATGCTGTGTGCCGGATACAAGAATGA
GAGCTATGATGCCTGCAAGGGTGACAGTGGGGGGCCTCTGGTCTGCACCCCAGAGCCTGGTGAGAAGT
GGTACCAGGTGGGCATCATAAGCTGGGGAAAGAGCTGTGGAGAGAAGAACACCCCAGGGATATACACC
TCGTTGGTGAACTACAACCTCTGGATCGAGAAAGTGACCCAGCTAGAGGGCAGGCCCTTCAATGCAGA
GAAAAGGAGGACTTCTGTCAAACAGAAACCTATGGGCTCCCCAGTCTCGGGAGTCCCAGAGCCAGGCA
GCCCCAGATCCTGGCTCCTGCTCTGTCCCCTGTCCCATGTGTTGTTCAGAGCTATTTTGTACCTCGAG

| | | | |
|---|---|---|---|
| NOV19j, CG51752-05 | SEQ ID NO: 262 | 267 aa | MW at 29440.8kD |
| Protein Sequence | | | |

MGFRFLGTANSAAFETSLPLPLAPLWFSATSPEELSVVLGTNDLTSPSMEIKEVASIILHKDFKRANN
DNDIALLLLASPIKLDDLKVPICLPTQPGPATWRECWVAGWGQTNAADKNSVKTDLMKAPMVIMDWEE
CSKMFPKLTKNNLCAGYKNESYDACKGDSGGPLVCTPEPGEKWYQVGIISWGKSCGEKNTPGIYTSLV
NYNLWIEKVTQLEGRPFMAEKRRTSVKQKPMGSPVSGVPEPGSPRSWLLLCPLSHVLFRAILY

| | | |
|---|---|---|
| NOV19k, CG51752-06 | SEQ ID NO: 263 | 729 bp |
| DNA Sequence | ORF Start: at 7 | ORF Stop: at 724 |

GGATCCGCCACCAGTCCAGAAGAACTGAGTGTCGTGCTGGGGACCAACGACTTAACTAGCCCATCCAT
GGAAATAAAGGAGGTCGCCAGCATCATTCTTCACAAAGACTTTAAGAGAGCCAACATGGACAATGACA
TTGCCTTGCTGCTGCTGGCTTCGCCCATCAAGCTCGATGACCTGAAGGTGCCCATCTGCCTCCCCACG
CAGCCCGGCCCTGCCACATGGCGCGAATGCTGGGTGGCAGGTTGGGGCCAGACCAATGCTGCTGACAA
AAACTCTGTGAAAACGGATCTGATGAAAGTGCCAATGGTCATCATGGACTGGGAGGAGTGTTCAAAGA
TGTTTCCAAAACTTACCAAAAATATGCTGTGTGCCGGATACAAGAATGAGAGCTATGATGCCTGCAAG
GGTGACAGTGGGGGGCCTCTGGTCTGCACCCCAGAGCCTGGTGAGAAGTGGTACCAGGTGGGCATCAT
CAGCTGGGGAAAGAGCTGTGGAGAGAAGAACACCCCAGGGATATACACCTCGTTGGTGAACTACAACC
TCTGGATCGAGAAAGTGACCCAGCTAGAGGGCAGGCCCTTCAATGCAGAGAAAAGGAGGACCTCTGTC
AAACAGAAACCTATGGGCTCCCCAGTCTCGGGAGTCCCAGAGCCAGGCAGCCCCAGATCCTGGCTCCT
GCTCTGTCCCCTGTCCCATGTGTTGTTCAGAGCTATTTTGTACCTCGAG

| | | | |
|---|---|---|---|
| NOV19k, CG51752-06 | SEQ ID NO: 264 | 239 aa | MW at 26444.4kD |
| Protein Sequence | | | |

ATSPEELSVVLGTNDLTSPSMEIKEVASIILHKDFKRANMDNDIALLLLASPIKLDDLKVPICLPTQP
GPATWRECWVAGWGQTNAADKNSVKTDLMKVPMVIMDWEECSKMFPKLTKNMLCAGYKNESYDACKGD
SGGPLVCTPEPGEKWYQVGIISWGKSCGEKNTPGIYTSLVNYNLWIEKVTQLEGRPFNAEKRRTSVKQ
KPMGSPVSGVPEPGSPRSWLLLCPLSHVLFRAILY

| | | |
|---|---|---|
| NOV19l, CG51752-07 | SEQ ID NO: 265 | 816 bp |
| DNA Sequence | ORF Start: ATG at 10 | ORF Stop: at 811 |

GGATCCACCATGGGATTTAGGTTTCTGGGCACAGCCAATTCTGCCACTTTTGAGACTTCCCTTCCCCT
TCCACTTGCCCCTCTCTGGTTCTCTGCCACCAGTCCAGAAGAACTGAGTGTCGTGCTGGGGACCAACG
ACTTAACTAGCCCATCCATGGAAATAAAGGAGGTCGCCAGCATCATTCTTCACAAAGACTTTAAGAGA
GCCAACATGGACAATGACATTGCCTTGCTGCTGCTGGCTTCGCCCATCAAGCTCGATGACCTGAAGGT
GCCCATCTGCCTCCCCACGCAGCCCGGCCCTGCCACATGGCGCGAATGCTGGGTGGCAGGTTGGGGCC
AGACCAATGCTGCTGACAAAAACTCTGTGAAAACGGATCTGATGAAAGTGCCAATGGTCATCATGGAC
TGGGAGGAGTGTTCAAAGATGTTTCCAAAACTTACCAAAAATATGCTGTGTGCCGGATACAAGAATGA
GAGCTATGATGCCTGCAAGGGTGACAGTGGGGGGCCTCTGGTCTGCACCCCAGAGCCTGGTGAGAAGT
GGTACCAGGTGGGCATCATCAGCTGGGGAAAGAGCTGTGGAGAGAAGAACACCCCAGGGATATACACC
TCGTTGGTGAACTACAACCTCTGGATCGAGAAAGTGACCCAGCTAGAGGGCAGGCCCTTCAATGCAGA
GAAAAGGAGGACCTCTGTCAAACAGAAACCTATGGGCTCCCCAGTCTCGGGAGTCCCAGAGCCAGGCA
GCCCCAGATCCTGGCTCCTGCTCTGTCCCCTGTCCCATGTGTTGTTCAGAGCTATTTTGTACCTCGAG

| | | | |
|---|---|---|---|
| NOV19l, GG51752-07 | SEQ ID NO: 266 | 267 aa | MW at 29498.9kD |
| Protein Sequence | | | |

MGFRFLGThNSATFETSLPLPLAPLWFSATSPEELSVVLGTNDLTSPSMEIKEVASIILHKDFKRANM
DNDIALLLLASPIKLDDLKVPICLPTQPGPATWRECWVAGWGQTNAADKNSVKTDLMKVPMVIMDWEE
CSKMFPKLTKNMLCAGYKNESYDACKGDSGGPLVCTPEPGEKWYQVGIISWGKSCGEKNTPGIYTSLV
NYNLWIEKVTQLEGRPFNAEKRRTSVKQKPMGSPVSGVPEPGSPRSWLLLCPLSHVLFRAILY

| | | |
|---|---|---|
| NOV19m, SNP13374584 of | SEQ ID NO: 267 | 1042 bp |
| CG51752-02, DNA Sequence | ORF Start: ATG at 207 | ORF Stop: TGA at 1008 |
| | SNP Pos: 243 | SNP Change: A to G |

AGAGTGCTCTAAACCCAGCTCGGCCTTTGCTGTATTAGACAGAAGCACCTCATTCATATCCCTGGGGC
CCCTGATGGTGCAGTGGTCTGGCTGTGGTCTGCACACCAGCTATTCTGTTTTGTTTTGTTTTGTTTTT
TCCTACCTTTTTCCAATCCTCACACCTTCTGATCAACAGCCCCAGTAGGGTTTAAAGGTCCTAGAGCT
ACATGGGATTTAGGTTTCTGGGCACAGCCAATTCTGCCGCTTTTGAGACTTCCCTTCCCCTTCCACTT
GCCCCTCTCTGGTTCTCTGCCACCAGTCCAGAAGAACTGAGTGTCGTGCTGGGGACCAACGACTTAAC
TAGCCCATCCATGGAAATAAAGGAGGTCGCCAGCATCATTCTTCACAAAGACTTTAAGAGAGCCAACA
TGGACAATGACATTGCCTTGCTGCTGCTGGCTTCGCCCATCAAGCTCGATGACCTGAAGGTGCCCATC
TGCCTCCCCACGCAGCCCGGCCCTGCCACATGGCGCGAATGCTGGGTGGCAGGTTGGGGCCAGACCAA
TGCTGCTGACAAAAACTCTGTGAAAACGGATCTGATGAAAGTGCCAATGGTCATCATGGACTGGGAGG
AGTGTTCAAAGATGTTTCCAAAACTTACCAAAAATATGCTGTGTGCCGGATACAAGAATGAGAGCTAT
GATGCCTGCAAGGGTGACAGTGGGGGGCCTCTGGTCTGCACCCCAGAGCCTGGTGAGAAGTGGTACCA
GGTGGGCATCATCAGCTGGGGAAAGAGCTGTGGAGAGAAGAACACCCCAGGGATATACACCTCGTTGG
TGAACTACAACCTCTGGATCGAGAAAGTGACCCAGCTAGAGGGCAGGCCCTTCAATGCAGAGAAAAGG
AGGACTTCTGTCAAACAGAAACCTATGGGCTCCCCAGTCTCGGGAGTCCCAGAGCCAGGCAGCCCCAG
ATCCTGGCTCCTGCTCTGTCCCCTGTCCCATGTGTTGTTCAGAGCTATTTTGTACTGATAATAAAATA
GAGGCTATTCTTTCAACCGAAA

| | | | |
|---|---|---|---|
| NOV19m, SNP13374584 of | SEQ ID NO: 268 | 267 aa | MW at 29468.8kD |
| CG51752-02, Protein | SNP Pos: 13 | | SNP Change: Thr to Ala |
| Sequence | | | |

MGFRFLGTANSAAFETSLPLPLAPLWFSATSPEELSVVLGTNDLTSPSMEIKEVASIILHKDFKRANM
DNDIALLLLASPIKLDDLKVPICLPTQPGPATWRECWVAGWGQTNAADKNSVKTDLMKVPMVIMDWEE
CSKMFPKLTKNMLCAGYKNESYDACKGDSGGPLVCTPEPGEKWYQVGIISWGKSCGEKNTPGIYTSLV
NYNLWIEKVTQLEGRPFNAEKRRTSVKQKPMGSPVSGVPEPGSPRSWLLLCPLSHVLFRAILY

| | | |
|---|---|---|
| NOV19n, SNP13374585 of | SEQ ID NO: 269 | 1042 bp |
| CG51752-02, DNA Sequence | ORF Start: ATG at 207 | ORF Stop: TGA at 1008 |
| | SNP Pos: 586 | SNP Change: T to C |

AGAGTGCTCTAAACCCAGCTCGGCCTTTGCTGTATTAGACAGAAGCACCTCATTCATATCCCTGGGGC
CCCTGATGGTGCAGTGGTCTGGCTGTGGTCTGCACACCAGCTATTCTGTTTTGTTTTGTTTTGTTTTT
TCCTACCTTTTTCCAATCCTCACACCTTCTGATCAACAGCCCCAGTAGGGTTTAAAGGTCCTAGAGCT
ACATGGGATTTAGGTTTCTGGGCACAGCCAATTCTGCCACTTTTGAGACTTCCCTTCCCCTTCCACTT
GCCCCTCTCTGGTTCTCTGCCACCAGTCCAGAAGAACTGAGTGTCGTGCTGGGGACCAACGACTTAAC
TAGCCCATCCATGGAAATAAAGGAGGTCGCCAGCATCATTCTTCACAAAGACTTTAAGAGAGCCAACA
TGGACAATGACATTGCCTTGCTGCTGCTGGCTTCGCCCATCAAGCTCGATGACCTGAAGGTGCCCATC
TGCCTCCCCACGCAGCCCGGCCCTGCCACATGGCGCGAATGCTGGGTGGCAGGTTGGGGCCAGACCAA
TGCTGCTGACAAAAACTCTGTGAAAACGGATCTGATGAAAGCGCCAATGGTCATCATGGACTGGGAGG
AGTGTTCAAAGATGTTTCCAAAACTTACCAAAAATATGCTGTGTGCCGGATACAAGAATGAGAGCTAT
GATGCCTGCAAGGGTGACAGTGGGGGGCCTCTGGTCTGCACCCCAGAGCCTGGTGAGAAGTGGTACCA
GGTGGGCATCATCAGCTGGGGAAAGAGCTGTGGAGAGAAGAACACCCCAGGGATATACACCTCGTTGG
TGAACTACAACCTCTGGATCGAGAAAGTGACCCAGCTAGAGGGCAGGCCCTTCAATGCAGAGAAAAGG
AGGACTTCTGTCAAACAGAAACCTATGGGCTCCCCAGTCTCGGGAGTCCCAGAGCCAGGCAGCCCCAG
ATCCTGGCTCCTGCTCTGTCCCCTGTCCCATGTGTTGTTCAGAGCTATTTTGTACTGATAATAAAATA
GAGGCTATTCTTTCAACCGAAA

| | | | |
|---|---|---|---|
| NOV19n, SNP13374585 of | SEQ ID NO: 270 | 267 aa | MW at 29470.8kD |
| CG51752-02, Protein | SNP Pos: 127 | | SNP Change: Val to Ala |
| Sequence | | | |

MGFRFLGTANSATFETSLPLPLAPLWFSATSPEELSVVLGTNDLTSPSMEIKEVASIILHKDFKRANM
DNDIALLLLASPIKLDDLKVPICLPTQPGPATWRECWVAGWGQTNAADKNSVKTDLMKAPMVIMDWEE
CSKMFPKLTKNMLCAGYKNESYDACKGDSGGPLVCTPEPGEKWYQVGIISWGKSCGEKNTPGIYTSLV
NYNLWIEKVTQLEGRPFNAEKRRTSVKQKPMGSPVSGVPEPGSPRSWLLLCPLSHVLFRAILY

A ClustalW comparison of the above protein sequences yields the following sequence alignment shown in Table 19B.

TABLE 19B
Comparison of the NOV19 protein sequences.

NOV19a ---MGFRFLGTANSATFETSLPLPLAPLWFSATSPEELSVVLGTNDLTSPSMEIKEVASI
NOV19b ---MGFRFLGTANSATFETSLPLPLAPLWFSATSPEELSVVLGTNDLTSPSMEIKEVASI
NOV19c GSTMGFRFLGTANSAAFETSLPLPLAPLWFSATSPEELSVVLGTNDLTSPSMEIKEVASI
NOV19d GSTMGFRFLGTANSAAFETSLPLPLAPLWFSATSPEELSVVLGTNDLTSPSMEIKEVASI
NOV19e -----------------------------GSATSPEELSVVLGTNDLTSPSMEIKEVASI
NOV19f -----------------------------GSATSPEELSVVLGTNDLTSPSMEIKEVASI
NOV19g ---MGFRFLGTANSATFEASLPLPLAPLWFSAPSPEELSVVLGTNDLTSPSMEIKEVASI
NOV19h GSTMGFRFLGTANSATFETSLPLPLAPLWFSATSPEELSVVLGTNDLTSPSMEIKEVASI
NOV19i ---MGFRFLGTANSATFETSLPLPLAPLWFSATSPEELSVVLGTNDLTSPSMEIKEVASI
NOV19j ---MGFRFLGTANSAAFETSLPLPLAPLWFSATSPEELSVVLGTNDLTSPSMEIKEVASI
NOV19k -------------------------------ATSPEELSVVLGTNDLTSPSMEIKEVASI
NOV19l ---MGFRFLGTANSATFETSLPLPLAPLWFSATSPEELSVVLGTNDLTSPSMEIKEVASI
NOV19a ILHKDFKRANMDNDIALLLLASPIKLDDLKVPICLPTQPGPATWRECWVAGWGQTNAADK
NOV19b ILHKDFKRANMDNDIALLLLASPIKLDDLKVPICLPTQPGPATWRECWVAGWGQTNAADK
NOV19c ILHKDFKRANMDNDIALLLLASPIKLDDLKVPICLPTQPGPATWRECWVAGWGQTNAADK
NOV19d ILHKDFKRANMDNDIALLLLASPIKLDDLKVPICLPTQPGPATWRECWVAGWGQTNAADK
NOV19e ILHKDFKRANMDNDIALLLLASPIKLDDLKVPICLPTQPGPATWRECWVAGWGQTNAADK
NOV19f ILHKDFKRANMDNDIALLLLASPIKLDDLKVPICLPTQPGPATWRECWVAGWGQTNAADK
NOV19g ILHKDFKRANNDNDIALLLLASPIKLDDLKVPICLPTQPGPATWRECWVAGWGQTNAADK
NOV19h ILHKDFKRANMDNDIALLLLASPIKLDDLKVPICLPTQPGPATWRECWVAGWGQTNAADK
NOV19i ILHKDFKRANMDNDIALLLLASPIKLDDLKVPICLPTQPGPATWRECWVAGWGQTNAADK
NOV19j ILHKDFKRANMDNDIALLLLASPIKLDDLKVPICLPTQPGPATWRECWVAGWGQTNAADK
NOV19k ILHKDFKRANMDNDIALLLLASPIKLDDLKVPICLPTQPGPATWRECWVAGWGQTNAADK
NOV19l ILHKDFKRANMDNDIALLLLASPIKLDDLKVPICLPTQPGPATWRECWVAGWGQTNAADK
NOV19a NSVKTDLMKVPMVIMDWEECSKMFPKLTKNMLCAGYKNESYDACKGDSGGPLVCTPEPGE
NOV19b NSVKTDLMKAPMVIMDWEECSKMFPKLTKNMLCAGYKNESYDACKGDSGGPLVCTPEPGE
NOV19c NSVKTDLMKAPMVIMDWEECSKMFPKLTKNMLCAGYKNESYDACKGDSGGPLVCTPEPGE
NOV19d NSVKTDLMKAPMVIMDWEECSKMFPKLTKNMLRAGYKNESYDACKGDSGGPLVCTPEPGE
NOV19e NSVKTDLMKAPMVIMDWEECSKMFPKLTKNMLCAGYKNESYDACKGDSGGPLVCTPEPGE
NOV19f NSVKTDLMKVPMVIMDWEECSKMFPKLTKNMLCAGYKNESYDACKGDSGGPLVCTPEPGE
NOV19g NSVKTDLMKAPMVIMDWEECSKMFPKLTKNMLCAGYKNESYDACKGDSGGPLVCTPEPGE
NOV19h NSVKTDLMKVPMVIMDWEECSKMFPKLTKNMLCAGYKNESYDACKGDSGGPLVCTPEPGE
NOV19i NSVKTDLMKVPMVIMDWEECSKMFPKLTKNMLCAGYKNESYDACKGDSGGPLVCTPEPGE
NOV19j NSVKTDLMKAPMVIMDWEECSKMFPKLTKNMLCAGYKNESYDACKGDSGGPLVCTPEPGE
NOV19k NSVKTDLMKVPMVIMDWEECSKMFPKLTKNMLCAGYKNESYDACKGDSGGPLVCTPEPGE
NOV19l NSVKTDLMKVPMVIMDWEECSKMFPKLTKNMLCAGYKNESYDACKGDSGGPLVCTPEPGE
NOV19a KWYQVGIISWGKSCGEKNTPGIYTSLVNYNLWIEKVTQLEGRPFNAEKRRTSVKQKPMGS
NOV19b KWYQVGIITWGKSCGEKNTPGIYTSLVNYNLWIEKVTQLEGRPFNAEKRRTSVKQKPMGS
NOV19c KWYQVGIISWGKSCGEKNTPGIYTSLVNYNLWIEKVTQLEGRPFNAEKRRTSVKQKPMGS
NOV19d KWYQVGIISWGKSCGEKMTPGIYTSLVNYNLWIEKVTQLEGRPFNAEKRRTSVKQKPMGS
NOV19e KWYQVGIISWGKSCGEKNTPGIYTSLVNYNLWIEKVTQLEGRPFNAEKRRTSVKQKPMGS
NOV19f KWYQVGIISWGKSCGEKNTPGIYTSLVNYNLWIEKVTQLEGRPFNAEKRRTSVKQKPMGS
NOV19g KWYQVGIISWGKSCGEKNTPGIYTSLVNYNLWIEKVTQLEGRPFNAEKRRTSVKQKPMGS
NOV19h KWYQVGIISWGKSCGEKNTPGIYTSLVNYNLWIEKVTQLEGRPFNAEKRRTSVKQKPMGS
NOV19i KWYQVGIISWGKSCGDKNTPGIYTSLVNYNLWIEKVTQLGGRPFNAEKRRTSVKQKPMGS
NOV19j KWYQVGIISWGKSCGEKNTPGIYTSLVNYNLWIEKVTQLEGRPFNAEKRRTSVKQKPMGS
NOV19k KWYQVGIISWGKSCGEKNTPGIYTSLVNYNLWIEKVTQLEGRPFNAEKRRTSVKQKPMGS
NOV19l KWYQVGIISWGKSCGEKNTPGIYTSLVNYNLWIEKVTQLEGRPFNAEKRRTSVKQKPMGS
NOV19a PVSGVPEPGSPRSWLLLCPLSHVLFRAILY--
NOV19b PVSGVPEPGSPRSWLPLCPLSHVLFRAILY--
NOV19c PVSGVPEPGSPRSWLLLCPLSHVLFRAILYLE
NOV19d PVSGVPEPGSPRSWLLLCPLSHVLFRAILYLE
NOV19e PVSGVPEPGSPRSWLLLCPLSHVLFRAILYLE
NOV19f PVSGVPEPGSPRSWLLLCPLSHVLFRAILYLE
NOV19g PVSGVPEPGSPRSWLLLCPLSHVLFRAILY--
NOV19h PVSGVPEPGSPRSWLLLCPLSHVLFRAILYLE
NOV19i PVSGVPEPGSPRSWLLLCPLSHVLFRAILY--
NOV19j PVSGVPEPGSPRSWLLLCPLSHVLFRAILY--
NOV19k PVSGVPEPGSPRSWLLLCPLSHVLFRAILY--
NOV19l PVSGVPEPGSPRSWLLLCPLSHVLFRAILY--
NOV19a (SEQ ID NO: 244)
NOV19b (SEQ ID NO: 246)
NOV19c (SEQ ID NO: 248)
NOV19d (SEQ ID NO: 250)
NOV19e (SEQ ID NO: 252)
NQV19f (SEQ ID NO: 254)
NOV19g (SEQ ID NO: 256)
NOV19h (SEQ ID NO: 258)
NOV19i (SEQ ID NO: 260)
NOV19j (SEQ ID NO: 262)
NOV19k (SEQ ID NO: 264)
NOV19l (SEQ ID NO: 266)

Further analysis of the NOV19a protein yielded the following properties shown in Table 19C.

TABLE 19C
Protein Sequence Properties NOV19a

SignalP analysis: No Known Signal Sequence Indicated
PSORT II analysis:

| PSG: | a new signal peptide prediction method |
|---|---|
| | N-region: length 4; pos.chg 1; neg.chg 0 |
| | H-region: length 10; peak value 4.03 |
| | PSG score: −0.38 |
| GvH: | von Heijne's method for signal seq. recognition |
| | GvH score (threshold: −2.1): −3.06 |
| | possible cleavage site: between 32 and 33 |

>>> Seems to have no N-terminal signal peptide

| ALOM: | Klein et al's method for TM region allocation |
|---|---|
| | Init position for calculation: 1 |
| | Tentative number of TMS(s) for the threshold 0.5: 0 |
| | Number of TMS(s) for threshold 0.5: 0 |
| | PERIPHERAL Likelihood = 2.38 (at 72) |
| | ALOM score: 0.05 (number of TMSs: 0) |

MITDISC: discrimination of mitochondrial targeting seq

| | | | |
|---|---|---|---|
| R content: | 1 | Hyd Moment(75): | 8.51 |
| Hyd Moment(95): | 7.65 | G content: | 2 |
| D/E content: | 2 | S/T content: | 8 |
| Score: −4.16 | | | |

Gavel: prediction of cleavage sites for mitochondrial preseq

R-2 motif at 14 FRF|LG

NUCDISC: discrimination of nuclear localization signals

pat4: none
pat7: none
bipartite: none
content of basic residues: 10.5%
NLS Score: −0.47

KDEL: ER retention motif in the C-terminus: none
ER Membrane Retention Signals:

XXRR-like motif in the N-terminus: GFRF

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:

Bacterial regulatory proteins, gntR family signature
(PS00043):

*** found ***

VASIILHKDFKRANMDNDIALL at 54

NNCN: Reinhardt's method for Cytoplasmic/Nuclear discrimination

Prediction: cytoplasmic
Reliability: 70.6

COIL: Lupas's algorithm to detect coiled-coil regions
total: 0 residues
Final Results (k = 9/23):

```
43.5%: mitochondrial
34.8%: cytoplasmic
  8.7%: peroxisomal
  4.3%: vacuolar
  4.3%: nuclear
  4.3%: endoplasmic reticulum
```

>> prediction for CG51752-02 is mit (k = 23)

A search of the NOV19a protein against the Geneseq database, a proprietary database that contains sequences published in patents and patent publication, yielded several homologous proteins shown in Table 19D.

TABLE 19D
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU08686 | Human FCTR6b polypeptide | 1 . . . 267 | 267/267 (100%) | e-160 |
| | sequence - Homo sapiens, 267 | 1 . . . 267 | 267/267 (100%) | |
| | aa. [WO200166747-A2, 13-SEP- | | | |
| | 2001] | | | |
| AAU08685 | Human FCTR6a polypeptide | 1 . . . 267 | 265/267 (99%) | e-159 |
| | sequence - Homo sapiens, 267 | 1 . . . 267 | 266/267 (99%) | |
| | aa. [WO200166747-A2, 13-SEP- | | | |
| | 2001] | | | |
| AAU82737 | Amino acid sequence of novel | 32 . . . 267 | 235/236 (99%) | e-141 |
| | human protease #36 - Homo | 117 . . . 352 | 235/236 (99%) | |
| | sapiens, 352 aa. [WO200200860- | | | |
| | A2, 03-JAN-2002] | | | |
| AAB03160 | Human trypsin family serine | 32 . . . 267 | 233/236 (98%) | e-140 |
| | protease Tespec PRO-3 - Homo | 117 . . . 352 | 234/236 (98%) | |
| | sapiens, 352 aa. [WO200026352- | | | |
| | A1, 11-MAY-2000] | | | |
| AAU76529 | Human LP polypeptide #2 - | 32 . . . 249 | 214/218 (98%) | e-127 |
| | Homo sapiens, 356 aa. | 117 . . . 334 | 215/218 (98%) | |
| | [WO200216578-A2, 28-FEB- | | | |
| | 2002] | | | |

In a BLAST search of public sequence databases, the NOV19a protein was found to have homology to the proteins shown in the BLASTP data in Table 19E.

TABLE 19E
Public BLASTP Results for NOV19a

| | | NOV19a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC88680 | Sequence 22 from Patent | 1 . . . 267 | 267/267 (100%) | e-160 |
| | WO0166747 - Homo sapiens | 1 . . . 267 | 267/267 (100%) | |
| | (Human), 267 aa. | | | |
| CAC88679 | Sequence 20 from Patent | 1 . . . 267 | 265/267 (99%) | e-159 |
| | WO0166747 - Homo sapiens | 1 . . . 267 | 266/267 (99%) | |
| | (Human), 267 aa. | | | |
| Q9MZZ6 | Hypothetical 29.5 kDa protein - | 1 . . . 267 | 248/267 (92%) | e-146 |
| | Macaca fascicularis (Crab eating | 1 . . . 267 | 251/267 (93%) | |
| | macaque) (Cynomolgus | | | |
| | monkey), 267 aa. | | | |
| CAD28997 | Sequence 3 from Patent | 32 . . . 249 | 214/218 (98%) | e-127 |
| | WO0216578 - Homo sapiens | 117 . . . 334 | 215/218 (98%) | |
| | (Human), 356 aa. | | | |
| AAH49714 | 1700049K14Rik protein - Mus | 27 . . . 225 | 87/200 (43%) | 5e-45 |
| | musculus (Mouse), 321 aa. | 98 . . . 297 | 119/200 (59%) | |

PFam analysis indicates that the NOV19a protein contains the domains shown in the Table 19F.

TABLE 19F
Domain Analysis of NOV19a

| | | Identities/ | |
|---|---|---|---|
| Pfam | | Similarities | Expect |
| Domain | NOV19a Match Region | for the Matched Region | Value |
| trypsin | 5 . . . 210 | 75/267 (28%) | 8.6e-34 |
| | | 153/267 (57%) | |

### Example 20

The NOV20 clone was analyzed, and the nucleotide and encoded polypeptide sequences are shown in Table 20A.

TABLE 20A
NOV20 Sequence Analysis

| | | |
|---|---|---|
| NOV20a, GG51914-02 | SEQ ID NO: 271 | 3261 bp |
| DNA Sequence | ORF Start: ATG at 92 | ORF Stop: TGA at 3146 |

AGCGGGGCTCTGGGGTCTGGGGGCATTGCTCAGCGGTGCTAGGCTGGCGCGGCTTGAGCCGCCGCCGG
ACTGACAGCTCGGTCTGCGGACCATGGAGACCTGCGCCGGTCCACACCCGCTGCGCCTCTTCCTCTGC
CGGATGCAGCTCTGTCTCGCGCTGCTTTTGGGACCCTGGCGGCCTGGGACCGCCGAGGAAGTTATCCT
CCTGGATTCCAAAGCCTCCCAGGCCGAGCTGGGCTGGACTGCACTGCCAAGTAATGGGTGGGAGGAGA
TCAGCGGCGTGGATGAACACGACCGTCCCATCCGCACGTACCAAGTGTGCAATGTGCTGGAGCCCAAC
CAGGACAACTGGCTGCAGACTGGCTGGATAAGCCGTGGCCGCGGGCAGCGCATCTTCGTGGAACTGCA
GTTCACACTCCGTGACTGCAGCAGCATCCCTGGCGCCGCGGGTACCTGCAAGGAGACCTTCAACGTCT
ACTACCTGGAAACTGAGGCCGACCTGGGCCGTGGGCGTCCCCGCCTAGGCGGCAGCCGGCCCCGCAAA
ATCGACACGATCGCGGCGGACGAGAGCTTCACGCAGGGCGACCTGGGTGAGCGCAAGATGAAGCTGAA
CACAGAGGTGCGCGAGATCGGACCGCTCAGCCGGCGGGGTTTCCACCTGGCCTTTCAGGACGTGGGCG
CATGCGTGGCGCTTGTCTCGGTGCGCGTCTACTACAAGCAGTGCCGCGCCACCGTGCGGGGCCTGGCC
ACGTTCCCAGCCACCGCAGCCGAGAGCGCCTTCTCCACACTGGTGGAAGTGGCCGGAACGTGCGTGGC
GCACTCGGAAGGGGAGCCTGGCAGCCCCCCACGCATGCACTGCGGCGCCGACGGCGAGTGGCTGGTGC
CTGTGGGCCGCTGCAGCTGCAGCGCGGGATTCCAGGAGCGTGGTGACTTCTGCGAATGTCCCCCAGGG
TTTTACAAGGTGTCCCCGCGGCGGCCCCTCTGCTCACCGTGCCCAGAGCACAGCCGGGCCCTGGAAAA
CGCCTCCACCTTCTGCGTGTGCCAGGACAGCTATGCGCGCTCACCCACCGACCCGCCCTCGGCTTCCT
GCACCCGTCCGCCGTCGGCGCCGCGGGACCTGCAGTACAGCCTGAGCCGCTCGCCGCTGGTGCTGCGA
CTGCGCTGGCTGCCGCCGGCCGACTCGGGAGGCCGCTCGGACGTCACCTACTCGCTGCTGTGCCTGCG
CTGCGGCCGCGAGGGCCCGGCGGGCGCCTGCGAGCCGTGCGGGCCGCGCGTGGCCTTCCTACCGCGCC
AGGCAGGGCTGCGGGAGCGAGCCGCCACGCTGCTGCACCTGCGGCCCGGGGCGCGCTACACCGTGCGC
GTGGCCGTGCTCAACGGCGTCTCGTCAGCGCCCTGGGAGGAGGATGAGATCCGCAGGGACCGAGTGGA
ACCCCAGAGCGTGTCCCTGTCGTGGCGGGAGCCCATCCCTGCCGGAGCCCCTGGGGCCAATGACACGG
AGTACGAGATCCGATACTACGAGAAGGGTCAGAGTGAGCAGACTTACTCCATGGTGAAGACGGGGGCG
CCCACAGTCACCGTCACCAACCTGAAGCCGGCTACCCGCTACGTCTTTCAGATCCGGGCCGCTTCCCC
GGGGCCATCCTGGGAGGCCCAGAGTTTTAACCCCAGCATTGAAGTACAGACCCTGGGGGAGGCTGCCT
CAGGGTCCAGGGACCAGAGCCCCGCCATTGTCGTCACCGTAGTGACCATCTCGGCCCTCCTCGTCCTG
GGCTCCGTGATGAGTGTGCTGGCCATTTGGAGGAGGCCCTGCAGCTATGGCAAAGGAGGAGGGGATGC
CCATGATGAAGAGGAGCTGTATTTCCACTTCAAAGTCCCAACACGTCGCACATTCCTGGACCCCCAGA
GCTGTGGGGACCTGCTGCAGGCTGTGCATCTGTTCGCCAAGGAACTGGATGCGAAAAGCGTCACGCTG
GAGAGGAGCCTTGGAGGAGGCAAGCTGGGCGCCCAGGAAGCCTTGTCCCCATCTGGAAGCCTCACCCA
CTCTATAGGCCCCGCCCCTACTCTGTCCACACCTCTATCCGGGCGGTTTGGGGAGCTGTGCTGTGGCT
GCTTGCAGCTCCCCGGTCGCCAGGAGCTGCTCGTAGCCGTGCATATGCTGAGGGACAGCGCCTCCGAC
TCACAGAGGCTCGGCTTCCTGGCCGAGGCCCTCACGCTGGGCCAGTTTGACCATAGCCACATCGTGCG
GCTGGAGGGCGTTGTTACCCGAGGTAGGGGAAGCACCTTGATGATTGTCACCGAGTACATGAGCCATG
GGGCCCTGGACGGCTTCCTCAGGCGGCACGAGGGGCAGCTGGTGGCTGGGCAACTGATGGGGTTGCTG
CCTGGGCTGGCATCAGCCATGAAGTATCTGTCAGAGATGGGCTACGTTCACCGGGGCCTGGCAGCTCG
CCATGTGCTGGTCAGCAGCGACCTTGTCTGCAAGATCTCTGGCTTCGGGCGGGGCCCCCGGGACCGAT
CAGAGGCTGTCTACACCACTGGCCGGAGCCCAGCGCTATGGGCCGCTCCCGAGACACTTCAGTTTGGC
CACTTCAGCTCTGCCAGTGACGTGTGGAGCTTCGGCATCATCATGTGGGAGGTGATGGCCTTTGGGGA
GCGGCCTTACTGGGACATGTCTGGCCAAGACGTGATCAAGGCTGTGGAGGATGGCTTCCGGCTGCCAC
CCCCCAGGAACTGTCCTAACCTTCTGCACCGACTAATGCTCGACTGCTGGCAGAAGGACCCAGGTGAG
CGGCCCAGGTTCTCCCAGATCCACAGCATCCTGAGCAAGATGGTGCAGGACCCAGAGCCCCCCAAGTG
TGCCCTGACTACCTGTCCCAGGCCTCCCACCCCACTAGCGGACCGTGCCTTCTCCACCTTCCCCTCCT
TTGGCTCTGTGGGCGCGTGGCTGGAGGCCCTGGACCTGTGCCGCTACAAGGACAGCTTCGCGGCTGCT
GGCTATGGGAGCCTGGAGGCCGTGGCCGAGATGACTGCCCAGGACCTGGTGAGCCTAGGCATCTCTTT
GGCTGAACATCGAGAGGCCCTCCTCAGCGGGATCAGCGCCCTGCAGGCACGAGTGCTCCAGCTGCAGG
GCCAGGGGGTGCAGGTGTGAGTGGACCCCATTCTTCCAAGGCAGGACTCCGGTGGGGGTCCAGTCCCC
CAGCCCTGCCCAAGGACCGTGGCAAGCTGCGCTCCAGCAGTGTGGGAGGGAGCGCTCTCTTCCTC

| | | | |
|---|---|---|---|
| NOV20a, CG51914-02 | SEQ ID NO: 272 | 1018 aa | MW at 110851.8kD |
| Protein Sequence | | | |

METCAGPHPLRLFLCRNQLCLALLLGPWRPGTAEEVILLDSKASQAELGWTALPSNGWEEISGVDEHD
RPIRTYQVCNVLEPNQDNWLQTGWISRGRGQRIFVELQFTLRDCSSIPGAAGTCKETFNVYYLETEAD
LGRGRPRLGGSRPRKIDTIAADESFTQGDLGERKMKLNTEVREIGPLSRRGFHLAFQDVGACVALVSV
RVYYKQCRATVRGLATFPATAAESAFSTLVEVAGTCVAHSEGEPGSPPRMHCGADGEWLVPVGRCSCS
AGFQERGDFCECPPGFYKVSPRRPLCSPCPEHSRALENASTFCVCQDSYARSPTDPPSASCTRPPSAP
RDLQYSLSRSPLVLRLRWLPPADSGGRSDVTYSLLCLRCGREGPAGACEPCGPRVAFLPRQAGLRYYE
ATLLHLRPGARYTVRVAVLNGVSSAPWEEDEIRRDRVEPQSVSLSWREPIPAGAPGANDTEYEIRYYE
KGQSEQTYSMVKTGAPTVTVTNLKPATRYVFQIRAASPGPSWEAQSFNPSIEVQTLGEAASGSRDQSP
AIVVTVVTISALLVLGSVMSVLAIWRRPCSYGKGGGDAHDEEELYFHFKVPTRRTFLDPQSCGDLLQA
VHLFAKELDAKSVTLERSLGGGKLGAQEALSPSGSLTHSIGPAPTLSTPLSGRFGELCCGCLQLPGRQ
ELLVAVHMLRDSASDSQRLGFLAEALTLGQFDHSHIVRLEGVVTRGRGSTLMIVTEYMSHGALDGFLR
RHEGQLVAGQLMGLLPGLASAMKYLSEMGYVHRGLAARHVLVSSDLVCKISGFGRGPRDRSEAVYTTG
RSPALWAAPETLQFGHFSSASDVWSFGIIMWEVMAFGERPYWDMSGQDVIKAVEDGFRLPPPRNCPNL
LHRLMLDCWQKDPGERPRFSQIHSILSKMVQDPEPPKCALTTCPRPPTPLADRAFSTFPSFGSVGAWL
EALDLCRYKDSFAAAGYGSLEAVAEMTAQDLVSLGISLAEHREALLSGISALQARVLQLQGQGVQV

| | | |
|---|---|---|
| NOV20b, GG51914-01 | SEQ ID NO: 273 | 3003 bp |
| DNA Sequence | ORF Start: ATG at 1 | ORF Stop: TGA at 3001 |

ATGGTATTGACAACTGCTATACCAGCCTGGCTTCTTAGCTGTTCCCTCCCACTCTCATCCTGGGCCCA
CCATGCGACACCGCCCCTCCGTCTAGTAGTTATCCTCCTGGATTCCAAAGCCTCCCAGGCCGAGCTGG
GCTGGACTGCACTGCCAAGTAATGGGTGGGAGGAGATCAGCGGCGTGGATGAACACGACCGTCCCATC
CGCACGTACCAAGTGTGCAATGTGCTGGAGCCCAACCAGGACAACTGGCTGCAGACTGGCTGGATAAG
CCGTGGCCGCGGGCAGCGCATCTTCGTGGAACTGCAGTTCACACTCCGTGACTGCAGCAGCATCCCTG
GCGCCGCGGGTACCTGCAAGGAGACCTTCAACGTCTACTACCTGGAAACTGAGGCCGACCTGGGCCGT
GGGCGTCCCCGCCTAGGCGGCAGCCGGCCCCGCAAAATCGACACGATCGCGGCGGACGAGAGCTTCAC
GCAGGGCGACCTGGGTGAGCGCAAGATGAAGCTGAACACAGAGGTGCGCGAGATCGGACCGCTCAGCC
GGCGGGGTTTCCACCTGGCCTTTCAGGACGTGGGCGCATGCGTGGCGCTTGTCTCGGTGCGCGTCTAC
TACAAGCAGTGCCGCGCCACCGTGCGGGGCCTGGCCACGTTCCCAGCCACCGCAGCCGAGAGCGCCTT
CTCCACACTGGTGGAAGTGGCCGGAACGTGCGTGGCGCACTCGGAAGGGGAGCCTGGCAGCCCCCCAC
GCATGCACTGCGGCGCCGACGGCGAGTGGCTGGTGCCTGTGGGCCGCTGCAGCTGCAGCGCGGGATTC
CAGGAGCGTGGTGACTTCTGCGAATGTCCCCCAGGGTTTTACAAGGTGTCCCCGCGGCGGCCCCTCTG
CTCACCGTGCCCAGAGCACAGCCGGGCCCTGGAAAACGCCTCCACCTTCTGCGTGTGCCAGGACAGCT
ATGCGCGCTCACCCACCGACCCGCCCTCGGCTTCCTGCACCCGTCCGCCGTCGGCGCCGCGGGACCTG
CAGTACAGCCTGAGCCGCTCGCCGCTGGTGCTGCGACTGCGCTGGCTGCCGCCGGCCGACTCGGGAGG
CCGCTCGGACGTCACCTACTCGCTGCTGTGCCTGCGCTGCGGCCGCGAGGGCCCGGCGGGCGCCTGCG
AGCCGTGCGGGCCGCGCGTGGCCTTCCTACCGCGCCAGGCAGGGCTGCGGGAGCGAGCCGCCACGCTG
CTGCACCTGCGGCCCGGCGCGCGCTACACCGTGCGCGTGGCCGCGCTCAACGGCGTCTCGGGCCCGGC
GGCCGCCGCGGGAACCACCTACGCGCAGGTCACCGTCTCCACCGGGCCCTCAGCGCCCTGGGAGGAGG
ATGAGATCCGCAGGGAACCGAGTGGAACCCCAGAGCGTGTCCCTGTCGTGGCGGGAGCCCATCCTGCC
GGAGCCCCTGGGGCCAATGACACGGAGTACGAGATCCGATACTACGAGAAGCAGAGTGAGCAGACTTA
CTCCATGGTGAAGACAGGGGCGCCCACAGTCACCGTCACCAACCTGAAGCCGGCTACCCGCTACGTCT
TTCAGATCCGGGCCGCTTCCCCGGGGCCATCCTGGGAGGCCCAGAGTTTTAACCCCAGCATTGAAGTA
CAGACCCTGGGGGAGGCTGCCTCAGGGTCCAGGGACCAGAGCCCCGCCATTGTCGTCACCGTAGTGAC
CATCTCGGCCCTCCTCGTCCTGGGCTCCGTGATGAGTGTGCTGGCCATTTGGAGGAGGAGGCCCTGCA
GCTATGGCAAAGGAGGAGGGGATGCCCATGATGAAGAGGAGCTGTATTTCCACTGTAAAGTCCCAACA
CGTCGCACATTCCTGGACCCCCAGAGCTGTGGGGACCTGCTGCAGGCTGTGCATCTGTTCGCCAAGGA
ACTGGATGCGAAAAGCGTCACGCTGGAGAGGAGCCTTGGAGGAGGCAAGTTTGGGGAGCTGTGCTGTG
GCTGCTTGCAGCTCCCCGGTCGCCAGGAGCTGCTCGTAGCCGTGCACATGCTGAGGGACAGCGCCTCC
GACTCACAGAGGCTCGGCTTCCTGGCCGAGGCCCTCACGCTGGGCCAGTTTGACCATAGCCACATCGT
GCGGCTGGAGGGCGTTGTTACCCGAGGTAGGACCTTGATGATTGTCACCGAGTACATGAGCCATGGGG
CCCTGGACGGCTTCCTCAGGCACGAGGGGCAGCTGGTGGCTGGGCAACTGATGGGGTTGCTGCCTGGG
CTGGCATCAGCCATGAAGTATCTGTCAGAGATGGGCTACGTTCACCGGGGCCTGGCAGCTCGCCATGT
GCTGGTCAGCAGCGACCTTGTCTGCAAGATCTCTGGCTTCGGGCGGGGCCCCCGGGACCGATCAGAGG
CTGTCTACACCACTGGCCGGAGCCCAGCGCTATGGGCCGCTCCCGAGACACTTCAGTTTGGCCACTTC
AGCTCTGCCAGTGACGTGTGGAGCTTCGGCATCATCATGTGGGAGGTGATGGCCTTTGGGGAGCGGCC
TTACTGGGACATGTCTGGCCAAGACGTGAAGGCTGTGGAGGATGGCTTCCGGCTGCCACCCCCCAGGA
ACTGTCCTAACCTTCTGCACCGACTAATGCTCGACTGCTGGCAGAAGGACCCAGGTGAGCGGCCCAGG
TTCTCCCAGATCCACAGCATCCTGAGCAAGATGGTGCAGGACCCAGAGCCCCCCAAGTGTGCCCTGAC
TACCTGTCCCAGGCCTCCCACTCCACTAGCCGACCGTGCCTTCTCCACCTTCCCCTCCTTTGGCTCTG
TGGGCGCGTGGCTGGAGGCCCTGGACCTGTGCCGCTACAAGGACAGCTTCGCGGCTGCTGGCTATGGG
AGCCTGGAGGCCGTGGCCGAGATGACTGCCCAGGACCTGGTGAGCCTAGGCATCTCTTTGGCTGAACA
TCGAGAGGCCCTCCTCAGCGGGATCAGCGCCCTGCAGGCACGAGTGCTCCAGCTGCAGGGCCAGGGGG
TGCAGGTGTGA

| | | | |
|---|---|---|---|
| NOV20b, CG51914-01 | SEQ ID NO: 274 | 1000 aa | MW at 108840.5kD |
| Protein Sequence | | | |

MVLTTAIPAWLLSCSLPLSSWAHHATPPLRLVVILLDSKASQAELGWTALPSNGWEEISGVDEHDRPI
RTYQVCNVLEPNQDNWLQTGWISRGRGQRIFVELQFTLRDCSSIPGAAGTCKETFNVYYLETEADLGR
GRPRLGGSRPRKIDTIAADESFTQGDLGERKMKLNTEVREIGPLSRRGFHLAFQDVGACVALVSVRVY
YKQCRATVRGLATFPATAAESAFSTLVEVAGTCVAHSEGEPGSPPRMHCGADGEWLVPVGRCSCSAGF
QERGDFCECPPGFYKVSPRRPLCSPCPEHSRALENASTFCVCQDSYARSPTDPPSASCTRPPSAPRDL
QYSLSRSPLVLRLRWLPPADSGGRSDVTYSLLCLRCGREGPAGACEPCGPRVAFLPRQAGLRERAATL
LHLRPGARYTVRVAALNGVSGPAAAAGTTYAQVTVSTGPSAPWEEDEIRRDRVEPQSVSLSWREPIPA
GAPGANDTEYEIRYYEKQSEQTYSMVKTGAPTVTVTNLKPATRYVFQIRAASPGPSWEAQSFNPSIEV
QTLGEAASGSRDQSPAIVVTVVTISALLVLGSVMSVLAIWRRRPCSYGKGGGDAHDEEELYFHCKVPT
RRTFLDPQSCGDLLQAVHLFAKELDAKSVTLERSLGGGKFGELCCGCLQLPGRQELLVAVHMLRDSAS
DSQRLGFLAEALTLGQFDHSHIVRLEGVVTRGRTLMIVTEYMSHGALDGFLRHEGQLVAGQLMGLLPG
LASANKYLSEMGYVHRGLAARHVLVSSDLVCKISGFGRGPRDRSEAVYTTGRSPALWAAPETLQFGHF
SSASDVWSFGIIMWEVMAFGERPYWDMSGQDVKAVEDGFRLPPPRNCPNLLHRLMLDCWQKDPGERPR
FSQIHSILSKMVQDPEPPKCALTTCPRPPTPLADRAFSTFPSFGSVGAWLEALDLCRYKDSFAAAGYG
SLEAVAEMTAQDLVSLGISLABHREALLSGISALQARVLQLQGQGVQV

A ClustalW comparison of the above protein sequences yields the following sequence alignment shown in Table 20B.

TABLE 20B
Comparison of the NOV20 protein sequences.

NOV20a METCAGPHPLRLFLCRMQLCLALLLGPWRPGTAEEVILLDSKASQAELGWTALPSNGWEE
NOV20b -MVLTTAIPAWLLSC--SLPLSSWAHHATPPLRLVVILLDSKASQAELGWTALPSNGWEE
NOV20a ISGVDEHDRPIRTYQVCNVLEPNQDNWLQTGWISRGRGQRIFVELQFTLRDCSSIPGAAG
NOV20b ISGVDEHDRPIRTYQVCNVLEPNQDNWLQTGWISRGRGQRIFVELQFTLRDCSSIPGAAG
NOV20a TCKETFNVYYLETEADLGRGRPRLGGSRPRKIDTIAADESFTQGDLGERKMKLNTEVREI
NOV20b TCKETFNVYYLETEADLGRGRPRLGGSRPRKIDTIAADESFTQGDLGERKMKLNTEVREI
NOV20a GPLSRRGFHLAFQDVGACVALVSVRVYYKQCRATVRGLATFPATAAESAFSTLVEVAGTC
NOV20b GPLSRRGFHLAFQDVGACVALVSVRVYYKQCRATVRGLATFPATAAESAFSTLVEVAGTC
NOV20a VAHSEGEPGSPPRNHCGADGEWLVPVGRCSCSAGFQERGDFCECPPGFYKVSPRRPLCSP
NOV20b VAHSEGEPGSPPRMHCGADGEWLVPVGRCSCSAGFQERGDFCECPPGFYKVSPRRPLCSP
NOV20a CPEHSRALENASTFCVCQDSYARSPTDPPSASCTRPPSAPRDLQYSLSRSPLVLRLRWLP
NOV20b CPEHSRALENASTFCVCQDSYARSPTDPPSASCTRPPSAPRDLQYSLSRSPLVLRLRWLP
NOV20a PADSGGRSDVTYSLLCLRCGREGPAGACEPCGPRVAFLPRQAGLRERAATLLHLRPGARY
NOV20b PADSGGRSDVTYSLLCLRCGREGPAGACEPCGPRVAFLPRQAGLRERAATLLHLRPGARY
NOV20a TVRVAVLNGVS-------------------SAPWEEDEIRRDRVEPQSVSLSWREPIPAG
NOV20b TVRVAALNGVSGPAAAAGTTYAQVTVSTGPSAPWEEDEIRRDRVEPQSVSLSWREPIPAG
NOV20a APGANDTEYEIRYYEKGQSEQTYSMVKTGAPTVTVTNLKPATRYVFQIRAASPGPSWEAQ
NOV20b APGANDTEYEIRYYEK-QSEQTYSMVKTGAPTVTVTNLKPATRYVFQIRAASPGPSWEAQ
NOV20a SFNPSIEVQTLGEAASGSRDQSPAIVVTVVTISALLVLGSVMSVLAIWRR-PCSYGKGGG
NOV20b SFNPSIEVQTLGEAASGSRDQSPAIVVTVVTISALLVLGSVMSVLAIWRRRPCSYGKGGG
NOV20a DAHDEEELYFHFKVPTRRTFLDPQSCGDLLQAVHLFAKELDAKSVTLERSLGGGKLGAQE
NOV20b DAHDEEELYFHFKVPTRRTFLDPQSCGDLLQAVHLFAKELDAKSVTLERSLGGG------
NOV20a ALSPSGSLTHSIGPAPTLSTPLSGRFGELCCGCLQLPGRQELLVAVHMLRDSASDSQRLG
NOV20b ------------------------KFGELCCGCLQLPGRQELLVAVHMLRDSASDSQRLG
NOV20a FLAEALTLGQFDHSHIVRLEGVVTRGRGSTLMIVTEYMSHGALDGFLRRHEGQLVAGQLM
NOV20b FLAEALTLGQFDHSHIVRLEGVVTRGR--TLMIVTEYMSHGALDGFLR-HEGQLVAGQLM
NOV20a GLLPGLASAMKYLSEMGYVHRGLAARHVLVSSDLVCKISGFGRGPRDRSEAVYTTGRSPA
NOV20b GLLPGLASAMKYLSEMGYVHRGLAARHVLVSSDLVCKISGFGRGPRDRSEAVYTTGRSPA
NOV20a LWAAPETLQFGHFSSASDVWSFGIIMWEVMAFGERPYWDMSGQDVIKAVEDGFRLPPPRN
NOV20b LWAAPETLQFGHFSSASDVWSFGIIMWEVMAFGERPYWDMSGQD-VKAVEDGFRLPPPRN
NOV20a CPNLLHRLMLDCWQKDPGERPRFSQIHSILSKMVQDPEPPKCALTTCPRPPTPLADRAFS
NOV20b CPNLLHRLMLDCWQKDPGERPRFSQIHSILSKMVQDPEPPKCALTTCPRPPTPLADRAFS
NOV20a TFPSFGSVGAWLEALDLCRYKDSFAAAGYGSLEAVAEMTAQDLVSLGISLAEHREALLSG
NOV20b TFPSFGSVGAWLEALDLCRYWDSFAAAGYGSLEAVAEMTAQDLVSLGISLAEHREALLSG
NOV20a ISALQARVLQLQGQGVQV
NOV20b ISALQARVLQLQGQGVQV
NOV20a (SEQ ID NO: 272)
NOV20b (SEQ ID NO: 274)

Further analysis of the NOV20a protein yielded the following properties shown in Table 20C.

TABLE 20C
Protein Sequence Properties NOV20a

SignalP analysis: Cleavage site between residues 34 and 35
PSORT II analysis:

| PSG: | a new signal peptide prediction method |
|---|---|
| | N-region: length 11; pos.chg 1; neg.chg 1 |
| | H-region: length 4; peak value 0.56 |
| | PSG score: −3.84 |
| GvH: | von Heijne's method for signal seq. recognition |
| | GvH score (threshold: −2.1): 0.06 |
| | possible cleavage site: between 33 and 34 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 3 | |
| | Number of TMS(s) for threshold 0.5: 1 | |
| | INTEGRAL Likelihood = −9.08 | Transmembrane 546-562 |

| | PERIPHERAL Likelihood = 0.85 (at 754) |
|---|---|
| | ALOM score: −9.08 (number of TMSs: 1) |
| MTOP: | Prediction of membrane topology (Hartmann et al.) |
| | Center position for calculation: 553 |
| | Charge difference: 3.5 C( 1.5) - N(-2.0) |
| | C > N: C-terminal side will be inside |

>>> membrane topology: type 1b (cytoplasmic tail 546 to 1018)
MITDISC: discrimination of mitochondrial targeting seq

| | | | |
|---|---|---|---|
| R content: | 3 | Hyd Moment(75): | 5.96 |
| Hyd Moment(95): | 3.64 | G content: | 3 |
| D/E content: | 2 | S/T content: | 2 |
| Score: −5.44 | | | |

Gavel: prediction of cleavage sites for mitochondrial preseq

R-2 motif at 39 WRP|GT

NUCDISC: discrimination of nuclear localization signals

pat4: RPRK (4) at 148
pat7: none
bipartite: none
content of basic residues: 10.2%
NLS Score: −0.22

KDEL: ER retention motif in the C-terminus: none
ER Membrane Retention Signals: none
SKL: peroxisomal targeting signal in the C-terminus: none
PTS2: 2nd peroxisomal targeting signal: found

RLFLCRMQL at 11

VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 556
LL at 609
LL at 682
LL at 762
LL at 884
LL at 997

checking 63 PROSITE DNA binding motifs:

```
Leucine zipper pattern (PS00029):  *** found ***
    LSGRFGELCCGCLQLPGRQELL at 662
    LVSLGISLAEHREALLSGISAL at 983
```

none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | Prediction: cytoplasmic |
| | Reliability: 70.6 |
| COIL: | Lupas's algorithm to detect coiled-coil regions |
| | total: 0 residues |

--------------------------
Final Results (k = 9/23):

```
39.1%: nuclear
21.7%: cytoplasmic
17.4%: mitochondrial
  8.7%: vesicles of secretory system
  4.3%: vacuolar
  4.3%: peroxisomal
  4.3%: endoplasmic reticulum
```

>> prediction for CG51914-02 is nuc (k = 23)

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB98843 | Human NEPHA - Homo | 1 . . . 1018 | 984/1040 (94%) | 0.0 |
| | sapiens, 1008 aa. | 1 . . . 1008 | 984/1040 (94%) | |
| | [WO200283735-A1, 24-OCT- | | | |
| | 2002] | | | |
| AAE19158 | Human kinase polypeptide | 1 . . . 1018 | 973/1041 (93%) | 0.0 |
| | (PKIN-16) - Homo sapiens, | 1 . . . 1009 | 975/1041 (93%) | |
| | 1009 aa. [WO200208399-A2, | | | |
| | 31-JAN-2002] | | | |
| AAU03553 | Human protein kinase #53 - | 1 . . . 1018 | 973/1041 (93%) | 0.0 |
| | Homo sapiens, 1009 aa. | 1 . . . 1009 | 975/1041 (93%) | |
| | [WO200138503-A2, 31-MAY- | | | |
| | 2001] | | | |
| AAU76874 | Human EphA full length kinase - | 37 . . . 1018 | 948/1004 (94%) | 0.0 |
| | Homo sapiens, 974 aa. | 1 . . . 974 | 948/1004 (94%) | |
| | [WO200208253-A2, 31-JAN- | | | |
| | 2002] | | | |
| AAM47209 | Human NOV3 protein - Homo | 36 . . . 1018 | 946/1003 (94%) | 0.0 |
| | sapiens, 1000 aa. | 33 . . . 1000 | 946/1003 (94%) | |
| | [WO200174851-A2, 11-OCT- | | | |
| | 2001] | | | |

In a BLAST search of public sequence databases, the NOV20a protein was found to have homology to the proteins shown in the BLASTP data in Table 20E.

TABLE 20E
Public BLASTP Results for NOV20a

| | | NOV20a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD23752 | Sequence 1 from Patent | 37 . . . 1018 | 948/1004 (94%) | 0.0 |
| | WO0208253 - Homo sapiens | 1 . . . 974 | 948/1004 (94%) | |
| | (Human), 974 aa (fragment). | | | |
| CAD13085 | Sequence 5 from Patent | 36 . . . 1018 | 946/1003 (94%) | 0.0 |
| | WO0174851 - Homo sapiens | 33 . . . 1000 | 946/1003 (94%) | |
| | (Human), 1000 aa. | | | |
| CAD23753 | Sequence 3 from Patent | 37 . . . 1018 | 947/1052 (90%) | 0.0 |
| | WO0208253 - Homo sapiens | 1 . . . 1022 | 947/1052 (90%) | |
| | (Human), 1022 aa (fragment). | | | |
| CAD23754 | Sequence 5 from Patent | 37 . . . 663 | 625/647 (96%) | 0.0 |
| | WO0208253 - Homo sapiens | 1 . . . 647 | 625/647 (96%) | |
| | (Human), 647 aa (fragment). | | | |
| CAD23755 | Sequence 7 from Patent | 37 . . . 663 | 624/695 (89%) | 0.0 |
| | WO0208253 - Homo sapiens | 1 . . . 695 | 624/695 (89%) | |
| | (Human), 695 aa (fragment). | | | |

PFam analysis indicates that the NOV20a protein contains the domains shown in the Table 20F.

TABLE 20F
Domain Analysis of NOV20a

| | | Identities/ | |
|---|---|---|---|
| Pfam | | Similarities for | Expect |
| Domain | NOV20a Match Region | the Matched Region | Value |
| EPH_1bd | 35 . . . 211 | 116/178 (65%) | 8.8e-122 |
| | | 159/178 (89%) | |
| fn3 | 337 . . . 432 | 25/100 (25%) | 1.9e-05 |
| | | 65/100 (65%) | |
| fn3 | 437 . . . 517 | 23/88 (26%) | 7.3e-09 |
| | | 58/88 (66%) | |
| pkinase | 656 . . . 910 | 81/298 (27%) | 8.3e-38 |
| | | 176/298 (59%) | |
| SAM | 941 . . . 1005 | 27/68 (40%) | 7.1e-16 |
| | | 51/68 (75%) | |

### Example 21

The NOV21 clone was analyzed, and the nucleotide and encoded polypeptide sequences are shown in Table 21A.

TABLE 21A
NOV21 Sequence Analysis

| | | |
|---|---|---|
| NOV21a, CG51965-01 | SEQ ID NO: 275 | 9087 bp |
| DNA Sequence | ORF Start: ATG at 1 | ORF Stop: TGA at 9085 |

ATGGCGCCGCCGCCGCCGCCCGTGCTGCCCGTGCTGCTGCTCCTGGCCGCCGCCGCCGCCCTGCCGGC
GATGGGGCTGCGAGCGGCCGCCTGGGAGCCGCGCGTACCCGGCGGGACCCGCGCCTTCGCCCTCCGGC
CCGGCTGTACCTACGCGGTGGGCGCCGCTTGCACGCCCCGGGCGCCGCGGGAGCTGCTGGACGTGGGC
CGCGATGGGCGGCTGGCAGGACGTCGGCGCGTCTCGGGCGCGGGGCGCCCGCTGCCGCTGCAAGTCCG
CTTGGTGGCCCGCAGTGCCCCGACGGCGCTGAGCCGCCGCCTGCGGGCGCGCACGCACCTTCCCGGCT
GCGGAGCCCGTGCCCGGCTCTGCGGAACCGGTGCCCGGCTCTGCGGGGCGCTCTGCTTCCCCGTCCCC
GGCGGCTGCGCGGCCGCGCAGCATTCGGCGCTCGCAGCTCCGACCACCTTACCCGCCTGCCGCTGCCC
GCCGCGCCCCAGGCCCCGCTGTCCCGGCCGTCCCATCTGCCTGCCGCCGGGCGGCTCGGTCCGCCTGC
GTCTGCTGTGCGCCCTGCGGCGCGCGGCTGGCGCCGTCCGGGTGGGACTGGCGCTGGAGGCCGCCACC
GCGGGGACGCCCTCCGCGTCGCCATCCCCATCGCCGCCCCTGCCGCCGAACTTGCCCGAAGCCCGGGC
GGGGCCGGCGCGACGGGCCCGGCGGGGCACGAGCGGCAGAGGGAGCCTGAAGTTTCCGATGCCCAACT
ACCAGGTGGCGTTGTTTGAGAACGAACCGGCGGGCACCCTCATCCTCCAGCTGCACGCGCACTACACC
ATCGAGGGCGAGGAGGAGCGCGTGAGCTATTACATGGAGGGGCTGTTCGACGAGCGCTCCCGGGGCTA
CTTCCGAATCGACTCTGCCACGGGCGCCGTGAGCACGGACAGCGTACTGGACCGCGAGACCAAGGAGA
CGCACGTCCTCAGGGTGAAAGCCGTGGACTACAGTACGCCGCCGCGCTCGGCCACCACCTACATCACT
GTCTTGGTCAAAGACACCAACGACCACAGCCCGGTCTTCGAGCAGTCGGAGTACCGCGAGCGCGTGCG
GGAGAACCTGGAGGTGGGCTACGAGGTGCTGACCATCCGCGCCAGCGACCGCGACTCGCCCATCAACG
CCAACTTGCGTTACCGCGTGTTGGGGGGCGCGTGGGACGTCTTCCAGCTCAACGAGAGCTCTGGCGTG
GTGAGCACACGGGCGGTGCTGGACCGGGAGGAGGCGGCCGAGTACCAGCTCCTGGTGGAGGCCAACGA
CCAGGGGCGCAATCCGGGCCCGCTCAGTGCCACGGCCACCGTGTACATCGAGGTGGAGGACGAGAACG
ACAACTACCCCCAGTTCAGCGAGCAGAACTACGTGGTCCAGGTGCCCGAGGACGTGGGGCTCAACACG
GCTGTGCTGCGAGTGCAGGCCACGGACCGGGACCAGGGCCAGAACGCGGCCATTCACTACAGCATCCT
CAGCGGGAACGTGGCCGGCCAGTTCTACCTGCACTCGCTGAGCGGGATCCTGGATGTGATCAACCCCT
TGGATTTCGAGGATGTCCAGAAATACTCGCTGAGCATTAAGGCCCAGGATGGGGGCCGGCCCCCGCTC
ATCAATTCTTCAGGGGTGGTGTCTGTGCAGGTGCTGGATGTCAACGACAACGAGCCTATCTTTGTGAG
CAGCCCCTTCCAGGCCACGGTGCTGGAGAATGTGCCCCTGGGCTACCCCGTGGTGCACATTCAGGCGG
TGGACGCGGACTCTGGAGAGAACGCCCGGCTGCACTATCGCCTGGTGGACACGGCCTCCACCTTTCTG
GGGGGCGGCAGCGCTGGGCCTAAGAATCCTGCCCCCACCCCTGACTTCCCCTTCCAGATCCACAACAG
CTCCGGTTGGATCACAGTGTGTGCCGAGCTGGACCGCGAGGAGGTGGAGCACTACAGCTTCGGGGTGG
AGGCGGTGGACCACGGCTCGCCCCCCATGAGCTCCTCCACCAGCGTGTCCATCACGGTGCTGGACGTG
AATGACAACGACCCGGTGTTCACGCAGCCCACCTACGAGCTTCGTCTGAATGAGGATGCGGCCGTGGG
GAGCAGCGTGCTGACCCTGCAGGCCCGCGACCGTGACGCCAACAGTGTGATTACCTACCAGCTCACAG
GCGGCAACACCCGGAACCGCTTTGCACTCAGCAGCCAGAGAGGGGGCGGCCTCATCACCCTGGCGCTA
CCTCTGGACTACAAGCAGGAGCAGCAGTACGTGCTGGCGGTGACAGCATCCGACGGCACACGGTCGCA
CACTGCGCATGTCCTAATCAACGTCACTGATGCCAACACCCACAGGCCTGTCTTTCAGAGCTCCCATT
ACACAGTGAGTGTCAGTGAGGACAGGCCTGTGGGCACCTCCATTGCTACCCTCAGTGCCAACGATGAG
GACACAGGAGAGAATGCCCGCATCACCTACGTGATTCAGGACCCCGTGCCGCAGTTCCGCATTGACCC
CGACAGTGGCACCATGTACACCATGATGGAGCTGGACTATGAGAACCAGGTCGCCTACACGCTGACCA
TCATGGCCCAGGACAACGGCATCCCGCAGAAATCAGACACCACCACCCTAGAGATCCTCATCCTCGAT
GCCAATGACAATGCACCCCAGTTCCTGTGGGATTTCTACCAGGGTTCCATCTTTGAGGATGCTCCACC
CTCGACCAGCATCCTCCAGGTCTCTGCCACGGACCGGGACTCAGGTCCCAATGGGCGTCTGCTGTACA
CCTTCCAGGGTGGGGACGACGGCGATGGGGACTTCTACATCGAGCCCACGTCCGGTGTGATTCGCACC
CAGCGCCGGCTGGACCGGGAGAATGTGGCCGTGTACAACCTTTGGGCTCTGGCTGTGGATCGGGGCAG
TCCCACTCCCCTTAGCGCCTCGGTAGAAATCCAGGTGACCATCTTGGACATTAATGACAATGCCCCCA
TGTTTGAGAAGGACGAACTGGAGCTGTTTGTTGAGGAGAACAACCCAGTGGGGTCGGTGGTGGCAAAG
ATTCGTGCTAACGACCCTGATGAAGGCCCTAATGCCCAGATCATGTATCAGATTGTGGAAGGGGACAT
GCGGCATTTCTTCCAGCTGGACCTGCTCAACGGGGACCTGCGTGCCATGGTGGAGCTGGACTTTGAGG
TCCGGCGGGAGTATGTGCTGGTGGTGCAGGCCACGTCGGCTCCGCTGGTGAGCCGAGCCACGGTGCAC
ATCCTTCTCGTGGACCAGAATGACAACCCGCCTGTGCTGCCCGACTTCCAGATCCTCTTCAACAACTA
TGTCACCAACAAGTCCAACAGTTTCCCCACCGGCGTGATCGGCTGCATCCCGGCCCATGACCCCGACG
TGTCAGACAGCCTCAACTACACCTTCGTGCAGGGCAACGAGCTGCGCCTGTTGCTGCTGGACCCCGCC
ACGGGCGAACTGCAGCTCAGCCGCGACCTGGACAACAACCGGCCGCTGGAGGCGCTCATGGAGGTGTC
TGTGTCTGCAGATGGCATCCACAGCGTCACGGCCTTCTGCACCCTGCGTGTCACCATCATCACGGACG
ACATGCTGACCAACAGCATCACTGTCCGCCTGGAGAACATGTCCCAGGAGAAGTTCCTGTCCCCGCTG
CTGGCCCTCTTCGTGGAGGGGGTGGCCGCCGTGCTGTCCACCACCAAGGACGACGTCTTCGTCTTCAA
CGTCCAGAACGACACCGACGTCAGCTCCAACATCCTGAACGTGACCTTCTCGGCGCTGCTGCCTGGCG
GCGTCCGCGGCCAGTTCTTCCCGTCGGAGGACCTGCAGGAGCAGATCTACCTGAATCGGACGCTGCTG
ACCACCATCTCCACGCAGCGCGTGCTGCCCTTCGACGACGACATCTGCCTGCGCGAGCCCTGCGAGAA
CTACATGAAGTGCGTGTCCGTTCTGCGATTCGACAGCTCCGCGCCCTTCCTCAGCTCCACCACCGTGC
TCTTCCGGCCCATCCACCCCATCAACGGCCTGCGCTGCCGCTGCCCGCCCGGCTTCACCGGCGACTAC
TGCGAGACGGAGATCGACCTCTGCTACTCCGACCCGTGCGGCGCCAACGGCCGCTGCCGCAGCCGCGA
GGGCGGCTACACCTGCGAGTGCTTCGAGGACTTCACTGGAGAGCACTGTGAGGTGGATGCCCGCTCAG
GCCGCTGTGCCAACGGGGTGTGCAAGAACGGGGGCACCTGCGTGAACCTGCTCATCGGCGGCTTCCAC
TGCGTGTGTCCTCCTGGCGAGTATGAGAGGCCCTACTGTGAGGTGACCACCAGGAGCTTCCCGCCCCA
GTCCTTCGTCACCTTCCGGGGCCTGAGACAGCGCTTCCACTTCACCATCTCCCTCACGTTTGCCACTC
AGGAAAGGAACGGCTTGCTTCTCTACAACGGCCGCTTCAATGAGAAGCACGACTTCATCGCCCTGGAG
ATCGTGGACGAGCAGGTGCAGCTCACCTTCTCTGCAGGTGCAGGCGAGACAACAACGACCGTGGCACC
GAAGGTTCCCAGTGGTGTGAGTGACGGGCGGTGGCACTCTGTGCAGGTGCAGTACTACAACAAGGTAA
GATGGGCCCCACCACTTCCCCCTGGCCCCCAGCCCAATATTGGCCACCTGGGCCTGCCCCATGGGCCG
TCCGGGGAAAAGATGGCCGTGGTGACAGTGGATGATTGTGACACAACCATGGCTGTGCGCTTTGGAAA
GGACATCGGGAACTACAGCTGCGCTGCCCAGGGCACTCAGACCGGCTCCAAGAAGTCCCTGGATCTGA
CCGGCCCTCTACTCCTGGGGGGTGTCCCCAACCTGCCAGAAGACTTCCCAGTGCACAACCGGCAGTTC
GTGGGCTGCATGCGGAACCTGTCAGTCGACGGCAAAAATGTGGACATGGCCGGATTCATCGCCAACAA
TGGCACCCGGGAAGGCTGCGCTGCTCGGAGGAACTTCTGCGATGGGAGGCGGTGTCAGAATGGAGGCA
CCTGTGTCAACAGGTGGAATATGTATCTGTGTGAGTGTCCACTCCGATTCGGCGGGAAGAACTGTGAG
CAAGCCATGCCTCACCCCCAGCTCTTCAGCGGTGAGAGCGTCGTGTCCTGGAGTGACCTGAACATCAT
CATCTCTGTGCCCTGGTACCTGGGGCTCATGTTCCGGACCCGGAAGGAGGACAGCGTTCTGATGGAGG
CCACCAGTGGTGGGCCCACCAGCTTTCGCCTCCAGATCCTGAACAACTACCTCCAGTTTGAGGTGTCC
CACGGCCCCTCCGATGTGGAGTCCGTGATGCTGTCCGGGTTGCGGGTGACCGACGGGGAGTGGCACCA
CCTGCTGATCGAGCTGAAGAATGTTAAGGAGGACAGTGAGATGAAGCACCTGGTCACCATGACCTTGG
ACTATGGGATGGACCAGAACAAGGCAGATATCGGGGGCATGCTTCCCGGGCTGACGGTAAGGAGCGTG
GTGGTCGGAGGCGCCTCTGAAGACAAGGTCTCCGTGCGCCGTGGATTCCGAGGCTGCATGCAGGGAGT
GAGGATGGGGGGGACGCCCACCAACGTCGCCACCCTGAACATGAACAACGCACTCAAGGTCAGGGTGA
AGGACGGCTGTGATGTGGACGACCCCTGTACCTCGAGCCCCTGTCCCCCCAATAGCCGCTGCCACGAC
GCCTGGGAGGACTACAGCTGCGTCTGTGACAAAGGGTACCTTGGAATAAACTGTGTGGATGCCTGTCA
CCTGAACCCCTGCGAGAACATGGGGGCCTGCGTGCGCTCCCCCGGCTCCCCGCAGGGCTACGTGTGCG
AGTGTGGGCCCAGTCACTACGGGCCGTACTGTGAGAACAAACTCGACCTTCCGTGCCCCAGAGGCTGG
TGGGGGAACCCCGTCTGTGGACCCTGCCACTGTGCCGTCAGCAAAGGCTTTGATCCCGACTGTAATAA
GACCAACGGCCAGTGCCAATGCAAGGAGAATTACTACAAGCTCCTAGCCCAGGACACCTGTCTGCCCT
GCGACTGCTTCCCCCATGGCTCCCACAGCCGCACTTGCGACATGGCCACCGGGCAGTGTGCCTGCAAG
CCCGGCGTCATCGGCCGCCAGTGCAACCGCTGCGACAACCCGTTTGCCGAGGTCACCACGCTCGGCTG
TGAAGTGATCTACAATGGCTGTCCCAAAGCATTTGAGGCCGGCATCTGGTGGCCACAGACCAAGTTCG
GGCAGCCGGCTGCGGTGCCATGCCCTAAGGGATCCGTTGGAAATGCGGTCCGACACTGCAGCGGGGAG
AAGGGCTGGCTGCCCCCAGAGCTCTTTAACTGTACCACCATCTCCTTCGTGGACCTCAGGGCCATGAA
TGAGAAGCTGAGCCGCAATGAGACGCAGGTGGACGGCGCCAGGGCCCTGCAGCTGGTGAGGGCGCTGC
GCAGTGCTACACAGCACACGGGCACGCTCTTTGGCAATGACGTGCGCACGGCCTACCAGCTGCTGGGC
CACGTCCTTCAGCACGAGAGCTGGCAGCAGGGCTTCGACCTGGCAGCCACGCAGGACGCCGACTTTCA
CGAGGACGTCATCCACTCGGGCAGCGCCCTCCTGGCCCCAGCCACCAGGGCGGCGTGGGAGCAGATCC
AGCGGAGCGAGGGCGGCACGGCACAGCTGCTCCGGCGCCTCGAGGGCTACTTCAGCAACGTGGCACGC
AACGTGCGGCGGACGTACCTGCGGCCCTTCGTCATCGTCACCGCCAACATGGTTCTTGCTGTCGACAT
CTTTGACAAGTTCAACTTTACGGGAGCCAGGGTCCCGCGATTCGACACCATCCATGAAGAGTTCCCCA
GGGAGCTGGAGTCCTCCGTCTCCTTCCCAGCCGACTTCTTCAGACCACCTGAAGAAAAAGAAGGCCCC
CTGCTGAGGCCGGCTGGCCGGAGGACCACCCCGCAGACCACGCGCCCGGGGCCTGGCACCGAGAGGGA
GGCCCCGATCAGCAGGCGGAGGCGACACCCTGATGACGCTGGCCAGTTCGCCGTCGCTCTGGTCATCA
TTTACCGCACCCTGGGGCAGCTCCTGCCCGAGCGCTACGACCCCGACCGTCGCAGCCTCCGGTTGCCT
CACCGGCCCATCATTAATACCCCGATGGTGAGCACGCTGGTGTACAGCGAGGGGGCTCCGCTCCCGAG
ACCCCTGGAGAGGCCCGTCCTGGTGGAGTTCGCCCTGCTGGAGGTGGAGGAGCGAACCAAGCCTGTCT
GCGTGTTCTGGAACCACTCCCTGGCCGTTGGTGGGACGGGAGGGTGGTCTGCCCGGGGCTGCGAGCTC
CTGTCCAGGAACCGGACACATGTCGCCTGCCAGTGCAGCCACACAGCCAGCTTTGCGGTGCTCATGGA
TATCTCCAGGCGTGAGAACGGGGAGGTCCTGCCTCTGAAGATTGTCACCTATGCCGCTGTGTCCTTGT
CACTGGCAGCCCTGCTGGTGGCCTTCGTCCTCCTGAGCCTGGTCCGCATGCTGCGCTCCAACCTGCAC
AGCATTCACAAGCACCTCGCCGTGGCGCTCTTCCTCTCTCAGCTGGTGTTCGTGATTGGGATCAACCA
GACGGAAAACCCGTTTCTGTGCACAGTGGTTGCCATCCTCCTCCACTACATCTACATGAGCACCTTTG
CCTGGACCCTCGTGGAGAGCCTGCATGTCTACCGCATGCTGACCGAGGTGCGCAACATCGACACGGGG
CCCATGCGGTTCTACTACGTCGTGGGCTGGGGCATCCCGGCCATTGTCACAGGACTGGCGGTCGGCCT
GGACCCCCAGGGCTACGGGAACCCCGACTTCTGCTGGCTGTCGCTTCAAGACACCCTGATTTGGAGCT
TTGCGGGGCCCATCGGAGCTGTTATAATCATCAACACAGTCACTTCTGTCCTATCTGCAAAGGTTTCC
TGCCAAAGAAAGCACCATTATTATGGGAAAAAAGGGATCGTCTCCCTGCTGAGGACCGCATTCCTCCT
GCTGCTGCTCATCAGCGCCACCTGGCTGCTGGGGCTGCTGGCTGTGAACCGCGATGCACTGAGCTTTC
ACTACCTCTTCGCCATCTTCAGCGGCTTACAGGGCCCCTTCGTCCTCCTTTTCCACTGCGTGCTCAAC
CAGGAGGTCCGGAAGCACCTGAAGGGCGTGCTCGGCGGGAGGAAGCTGCACCTGGAGGACTCCGCCAC
CACCAGGGCCACCCTGCTGACGCGCTCCCTCAACTGCAACACCACCTTCGGTGACGGGCCTGACATGC
TGCGCACAGACTTGGGCGAGTCCACCGCCTCGCTGGACAGCATCGTCAGGGATGAAGGGATCCAGAAG
CTCGGCGTGTCCTCTGGGCTGGTGAGGGGCAGCCACGGAGAGCCAGACGCGTCCCTCATGCCCAGGAG
CTGCAAGGATCCCCCTGGCCACGATTCCGACTCAGATAGCGAGCTGTCCCTGGATGAGCAGAGCAGCT
CTTACGCCTCCTCACACTCGTCAGACAGCGAGGACGATGGGGTGGGAGCTGAGGAAAAATGGGACCCG
GCCAGGGGCGCCGTCCACAGCACCCCCAAAGGGGACGCTGTGGCCAACCACGTTCCGGCCGGCTGGCC
CGACCAGAGCCTGGCTGAGAGTGACAGTGAGGACCCCAGCGGCAAGCCCCGCCTGAAGGTGGAGACCA
AGGTCAGCGTGGAGCTGCACCGCGAGGAGCAGGGCAGTCACCGTGGAGAGTACCCCCCGGACCAGGAG
AGCGGGGGGCGCAGCCAGGCTTGCTAGCAGCCAGCCCCCAGAGCAGAGGAGCATCTTGAAAAATAAGT
CACCTACCCGCCGCCGCTGACGCTGACGGAGCAGACGCTGAAGGGCCGGCTCCGGGAGAAGCTGGCCG
ACTGTGAGCAGAGCCCCACATCCTCGCGCACGTCTTCCCTGGGCTCTGGCGGCCCCGACTGCGCCATC
ACAGTCAAGAGCCCTGGGAGGGAGCCGGGGCGTGACCACCTCAACGGGGTGGCCATGAATGTGCGCAC
TGGAGCGCCCAGGCCGATGGCTCCGACTCTGAGAAACCGTGA

| | | | |
|---|---|---|---|
| NOV21a, GG51965-01 | SEQ ID NO: 276 | 3028 aa | MW at 330865.9kD |
| Protein Sequence | | | |

MAPPPPPVLPVLLLLAAAAALPAMGLRAAAWEPRVPGGTRAFALRPGCTYAVGAACTPRAPRELLDVG
RDGRLAGRRRVSGAGRPLPLQVRLVARSAPTALSRRLRARTHLPGCGARARLCGTGARLCGALCFPVP
GGCAAAQHSALAAPTTLPACRCPPRPRPRCPGRPICLPPGGSVRLRLLCALRRAAGAVRVGLALEAAT
AGTPSASPSPSPPLPPNLPEARAGPARRARRGTSGRGSLKFPMPNYQVALFENEPAGTLILQLHAHYT
IEGEEERVSYYMEGLFDERSRGYFRIDSATGAVSTDSVLDRETKETHVLRVKAVDYSTPPRSATTYIT
VLVKDTNDHSPVFEQSEYRERVRENLEVGYEVLTIRASDRDSPINANLRYRVLGGAWDVFQLNESSGV
VSTRAVLDREEAAEYQLLVEANDQGRNPGPLSATATVYIEVEDENDNYPQFSEQNYVVQVPEDVGLNT
AVLRVQATDRDQGQNAAIHYSILSGNVAGQFYLHSLSGILDVINPLDFEDVQKYSLSIKAQDGGRPPL
INSSGVVSVQVLDVNDNEPIFVSSPFQATVLENVPLGYPVVHIQAVDADSGENARLHYRLVDTASTFL
GGGSAGPKNPAPTPDFPFQIHNSSGWITVCAELDREEVEHYSFGVEAVDHGSPPMSSSTSVSITVLDV
NDNDPVFTQPTYELRLNBDAAVGSSVLTLQARDRDANSVITYQLTGGNTRNRFALSSQRGGGLITLAL
PLDYKQEQQYVLAVTASDGTRSHTAHVLINVTDANTHRPVFQSSHYTVSVSEDRPVGTSIATLSANDE
DTGENARITYVIQDPVPQFRIDPDSGTMYTMMELDYENQVAYTLTIMAQDNGIPQKSDTTTLEILILD
ANDNAPQFLWDFYQGSIFEDAPPSTSILQVSATDRDSGPNGRLLYTFQGGDDGDGDFYIEPTSGVIRT
QRRLDRENVAVYNLWALAVDRGSPTPLSASVEIQVTILDINDNAPMFEKDELELFVEENNPVGSVVAK
IRANDPDEGPNAQIMYQIVEGDMRHFFQLDLLNGDLRAMVELDFEVRREYVLVVQATSAPLVSRATVH
ILLVDQNDNPPVLPDFOILFNNYVTNKSNSFPTGVIGCIPAHDPDVSDSLNYTFVQGNELRLLLLDPA
TGELQLSRDLDNNRPLEALMEVSVSADGIHSVTAFCTLRVTIITDDMLTNSITVRLENMSQEKFLSPL
LALFVEGVAAVLSTTKDDVFVFNVQNDTDVSSNILNVTFSALLPGGVRGQFFPSEDLQEQIYLNRTLL
TTISTQRVLPFDDNICLREPCENYMKCVSVLRFDSSAPFLSSTTVLFRPIHPINGLRCRCPPGFTGDY
CETEIDLCYSDPCGANGRCRSREGGYTCECFEDFTGEHCEVDARSGRCANGVCKNGGTCVNLLIGGFH
CVCPPGEYERPYCEVTTRSFPPQSFVTFRGLRQRFHFTISLTFATQERNGLLLYNGRFNEKHDFIALE
IVDEQVQLTFSAGAGETTTTVAPKVPSGVSDGRWHSVQVQYYNKVRWAPPLPPGPQPNIGHLGLPHGP
SGEKMAVVTVDDCDTTMAVRFGKDIGNYSCAAQGTQTGSKKSLDLTGPLLLGGVPNLPEDFPVNHRQF
VGCMRNLSVDGKNVDMAGFIANNGTREGCAARRNFCDGRRCQNGGTCVNRWNMYLCECPLRFGGKNCE
QAMPHPQLFSGESVVSWSDLNIIISVPWYLGLMFRTRKEDSVLMEATSGGPTSFRLQILNNYLQFEVS
HGPSDVESVMLSGLRVTDGEWHHLLIELKNVKEDSEMKHLVTMTLDYGMDQNKADIGGMLPGLTVRSV
VVGGASEDKVSVRRGFRGCMQGVRMGGTPTNVATLNMNNALKVRVKDGCDVDDPCTSSPCPPNSRCHD
AWEDYSCVCDKGYLGIMCVDACHLNPCENMGACVRSPGSPQGYVCECGPSHYGPYCENKLDLPCPRGW
WGNPVCGPCHCAVSKGFDPDCNKTNGQCQCKENYYKLLAQDTCLPCDCFPHGSHSRTCDMATGQCACK
PGVIGRQCNRCDNPFAEVTTLGCEVIYNGCPKAPEAGIWWPQTKFGQPAAVPCPKGSVGNAVRHCSGE
KGWLPPELFNCTTISFVDLRAMNEKLSRNETQVDGARALQLVRALRSATQHTGTLFGNDVRTAYQLLG
HVLQHESWQQGFDLAATQDADFHEDVIHSGSALLAPATRAAWEQIQRSEGGTAQLLRPLEGYFSNVAR
NVRRTYLRPFVIVTANMVLAVDIFDKFNFTGARVPRFDTIHEEFPRELESSVSFPADFFRPPEEKEGP
LLRPAGRRTTPQTTRPGPGTEREAPISRRRRHPDDAGQFAVALVIIYRTLGQLLPERYDPDRRSLRLP
HRPIINTPMVSTLVYSEGAPLPRPLERPVLVEFALLEVEERTKPVCVFWNHSLAVGGTGGWSARGCEL
LSRNRTHVACQCSHTASFAVLMDISRRENGEVLPLKIVTYAAVSLSLAALLVAFVLLSLVRMLRSNLH
SIHKHLAVALFLSQLVFVIGINQTENPFLCTVVAILLHYIYMSTFAWTLVESLHVYRMLTEVRNIDTG
PMRFYYVVGWGIPAIVTGLAVGLDPQGYGNPDFCWLSLQDTLIWSFAGPIGAVIIINTVTSVLSAKVS
CQRKHHYYGKKGIVSLLRTAFLLLLLISATWLLGLLAVNRDALSFHYLFAIFSGLQGPFVLLFHCVLN
QEVRKHLKGVLGGRKLHLEDSATTRATLLTRSLNCNTTFGDGPDMLRTDLGESTASLDSIVRDEGIQK
LGVSSGLVRGSHGEPDASLMPRSCKDPPGHDSDSDSELSLDEQSSSYASSHSSDSEDDGVGAEEKWDP
ARGAVHSTPKGDAVANHVPAGWPDQSLAESDSEDPSGKPRLKVETKVSVELHREEQGSHRGEYPPDQE
SGGAARLASSQPPEQRSILKNKVTYPPPLTLTEQTLKGRLREKLADCEQSPTSSRTSSLGSGGPDCAI
TVKSPGREPGRDHLNGVAMNVRTGSAQADGSDSEKP

| | | |
|---|---|---|
| NOV21b, 258076370 | SEQ ID NO: 277 | 1263 bp |
| DNA Sequence | ORF Start: at 1 | ORF Stop: end of sequence |

CTCAAGCTTGAAGTGATCTACAATGGCTGTCCCAAAGCATTTGAGGCCGGCATCTGGTGGCCACAGAC
CAAGTTCGGGCAGCCGGCTGCGGTGCCATGCCCTAAGGGATCCGTTGGAAATGCGGTCCGACACTGCA
GCGGGGAGAAGGGCTGGCTGCCCCCAGAGCTCTTTAACTGTACCACCATCTCCTTCGTGGACCTCAGG
GCCATGAATGAGAAGCTGAGCCGCAATGAGACGCAGGTGGACGGCGCCAGGGCCCTGCAGCTGGTGAG
GGCGCTGCGCAGTGCTACACAGCACACGGGCACGCTCTTTGGCAATGACGTGCGCACGGCCTACCAGC
TGCTGGGCCACGTCCTTCAGCACGAGAGCTGGCAGCAGGGCTTCGACCTGGCAGCCACGCAGGACGCC
GACTTTCACGAGGACGTCATCCACTCGGGCAGCGCCCTCCTGGCCCCAGCCACCAGGGCGGCGTGGGA
GCAGATCCAGCGGAGCGAGGGCGGCACGGCACAGCTGCTCCGGCGCCTCGAGGGCTACTTCAGCAACG
TGGCACGCAACGTGCGGCGGACGTACCTGCGGCCCTTCGTCATCGTCACCGCCAACATGATTCTTGCT
GTCGACATCTTTGACAAGTTCAACTTTACGGGAGCCAGGGTCCCGCGATTCGACACCATCCATGAAGA
GTTCCCCAGGGAGCTGGAGTCCTCCGTCTCCTTCCCAGCCGACTTCTTCAGACCACCTGAAGAAAAAG
AAGGCCCCCTGCTGAGGCCGGCTGGCCGGAGGACCACCCCGCAGACCACGCGCCCGGGGCCTGGCACC
GAGAGGGAGGCCCCGATCAGCAGGCGGAGGCGACACCCTGATGACGCTGGCCAGTTCGCCGTCGCTCT
GGTCATCATTTACCGCACCCTGGGGCAGCTCCTGCCCGAGCGCTACGACCCCGACCGTCGCAGCCTCC
GGTTGCCTCACCGGCCCATCATTAATACCCCGATGGTGAGCACGCTGGTGTACAGCGAGGGGGCTCCG
CTCCCGAGACCCCTGGAGAGGCCCGTCCTGGTGGAGTTCGCCCTGCTGGAGGTGGAGGAGCGAACCAA
GCCTGTCTGCGTGTTCTGGAACCACTCCCTGGCCGTTGGTGGGACGGGAGGGTGGTCTGCCCGGGGCT
GCGAGCTCCTGTCCAGGAACCGGACACATGTCGCCTGCCAGTGCAGCCACACAGCCAGCTTTGCGGTG
CTCATGGATATCTCCAGGCGTGAGAACGGGGAGAAGCTT

| | | | |
|---|---|---|---|
| NOV21b, 258076370 | SEQ ID NO: 278 | 421 aa | MW at47178.1kD |
| Protein Sequence | | | |

LKLEVIYNGCPKAFEAGIWWPQTKFGQPAAVPCPKGSVGNAVRHCSGEKGWLPPELFNCTTISFVDLR
AMNEKLSRNETQVDGARALQLVRALRSATQHTGTLFGNDVRTAYQLLGHVLQHESWQQGFDLAATQDA
DFHEDVIHSGSALLAPATRAAWEQIQRSEGGTAQLLRRLEGYFSNVARNVRRTYLRPFVIVTANMILA
VDIFDKFNFTGARVPRFDTIHEEFPRELESSVSFPADFFRPPEEKEGPLLRPAGRRTTPQTTRPGPGT
EREAPISRRRRHPDDAGQFAVALVIIYRTLGQLLPERYDPDRRSLRLPNRPIINTPMVSTLVYSEGAP
LPRPLERPVLVEFALLEVEERTKPVCVFWNHSLAVGGTGGWSARGCELLSRNRTHVACQCSHTASFAV
LMDISRRENGEKL

| | | |
|---|---|---|
| NOV21c, 317619862 | SEQ ID NO: 279 | 750 bp |
| DNA Sequence | ORF Start: at 1 | ORF Stop: end of sequence |

CGCAAGCTTGTCCTGCCTCTGAAGATTGTCACCTATGCCGCTGTGTCCTTGTCACTGGCAGCCCTGCT
GGTGGCCTTCGTCCTCCTGAGCCTGGTCCGCATGCTGCGCTCCAACCTGCACAGCATTCACAAGCACC
TCGCCGTGGCGCTCTTCCTCTCTCAGCTGGTGTTCGTGATTGGGATCAACCAGACGGAAAACCCGTTT
CTGTGCACAGTGGTTGCCATCCTCCTCCACTACATCTACATGAGCACCTTTGCCTGGACCCTCGTGGA
GAGCCTGCATGTCTACCGCATGCTGACCGAGGTGCGCAACATCGACACGGGGCCCATGCGGTTCTACT
ACGTCGTGGGCTGGGGCATCCCGGCCATTGTCACAGGACTGGCGGTCGGCCTGGACCCCCAGGGCTAC
GGGAACCCCGACTTCTGCTGGCTGTCGCTTCAAGACACCCTGATTTGGAGCTTTGCGGGGCCCATCGG
AGCTGTTATAATCATCAACACAGTCACTTCTGTCCTATCTGCAAAGGTTTCCTGCCAAAGAAAGCACC
ATTATTATGGGAAAAAAGGGATCGTCTCCCTGCTGAGGACCGCATTCCTCCTGCTGCTGCTCATCAGC
GCCACCTGGCTGCTGGGGCTGCTGGCTGTGAACCGCGACGCACTGAGCTTTCACTACCTCTTCGCCAT
CTTCAGCGGCTTACAGGGCCCCTTCGTCCTCCTTTTCCACTGCGTGCTCAACCAGGAGGTCAAGCTTG
CG

| | | | |
|---|---|---|---|
| NOV21c, 317619862 | SEQ ID NO: 280 | 250 aa | MW at 27799.9kD |
| Protein Sequence | | | |

RKLVLPLKIVTYAAVSLSLAALLVAFVLLSLVRMLRSNLHSIHKHLAVALFLSQLVFVIGINQTENPF
LCTVVAILLHYIYMSTFAWTLVESLHVYRMLTEVRNIDTGPMRFYYVVGWGIPAIVTGLAVGLDPQGY
GNPDFCWLSLQDTLIWSFAGPIGAVIIINTVTSVLSAKVSCQRKHHYYGKKGIVSLLRTAFLLLLLIS
ATWLLGLLAVNRDALSFHYLFAIFSGLQGPFVLLFHCVLNQEVKLA

| | | |
|---|---|---|
| NOV21d, 317460050 | SEQ ID NO: 281 | 2541 bp |
| DNA Sequence | ORF Start: at 1 | ORF Stop: end of sequence |

AAGCTTTACCAGGTGGCGTTGTTTGAGAACGAACCGGCGGGCACCCTCATCCTCCAGCTGCACGCGCA
CCACACCATCGAGGGCGAGGAGGAGCGCGTGAGCTATTACATGGAGGGGCTGTTCGACGAGCGCTCCC
GGGGCTACTTCCGAATCGACTCTGCCGCGGGCGCCGTGAGCACGGACAGCGTACTGGACCGCGAGACC
AAGGAGACGCACGTCCTCAGGGTGAAAGCCGTGGACTACAGTACGCCGCCGCGCTCGGCCACCACCTA
CATCACTGTCTTGGTCAAAGACACCAACGACCACAGCCCGGTCTTCGAGCAGTCGGAGTACCGCGAGC
GCGTGCGGGAGAACCTGGAGGTGGGCTACGAGGTGCTGACCATCCGCGCCAGCGACCGCGACTCGCCC
ATCAACGCCAACTTGCGTTACCGCGTGTTGGGGGGCGCGTGGGACGTCTTCCAGCTCAACGAGAGCTC
TGGCGTGGTGAGTACACGGGCGGTGCTGGACCGGGAGGAGGCGGCCGAGTACCAGCTGCTGGTGGAGG
CCAACGACCAGGGGCGCAATCCGGGCCCGCTCAGTGCCACGGCCACCGTGTACATCGAGGTGGAGGAC
GAGAACGACAACTACCCCCAGTTCAGCGAGCAGAACTACGTGGTCCAGGTGCCCGAGGACGTGGGGCT
CAACACGGCTGTGCTGCGAGTGCAGGCCACGGACCGGGACCAGGGCCAGAACGCGGCCATTCACTACA
GCATCCTCAGCGGGAACGTGGCCGGCCAGTTCTACCTGCACTCGCTGAGCGGGATCCTGGATGTGATC
AACCCCTTGGATTTCGAGGATGTCCAGAAATACTCGCTGAGCATTAAGGCCCAGGATGGGGGCCGGCC
CCCGCTCATCAATTCTTCAGGGGTGGTGTCTGTGCAGGTGCTGGATGTCAACGACAACGAGCCTATCT
TTGTGAGCAGCCCCTTCCAGGCCACGGTGCTGGAGAATGTGCCCCTGGGCTACCCCGTGGTGCACATT
CAGGCGGTGGACGCGGACTCTGGAGAGAACGCCCGGCTGCACTATCGCCTGGTGGACACGGCCTCCAC
CTTTCTGGGGGGCGGCAGCGCTGGGCCTAAGAATCCTGCCCCCACCCCTGACTTCCCCTTCCAGATCC
GCAACAGCTCCGGTTGGATCACAGTGTGTGCCGAGCTGGACCGCGAGGAGGTGGAGCACTACAGCTTC
GGGGTGGAGGCGGTGGACCACGGCTGGCCCCCCATGAGCTCCTCCACCAGCGTGTCCATCACGGTGCT
GGACGTGAATGACAACGACCCGGTGTTCACGCAGCCCACCTACGAGCTTCGTCTGAATGAGGATGCGG
CCGTGGGGAGCAGCGTGCTGACCCTGCAGGCCCGCGACCGTGACGCCAACAGTGTGATTACCTACCAG
CTCACAGGCGGCAACACCCGGAACCGCTTTGCACTCAGCAGCCAGAGAGGGGGCGGCCTCATCACCCT
GGCGCTACCTCTGGACTACAAGCAGGAGCAGCAGTACGTGCTGGCGGTGACAGCATCCGACGGCACAC
GGTCGCACACTGCGCATGTCCTAATCAACGTCACTGATGCCAACACCCACAGGCCTGTCTTTCAGAGC
TCCCATTACACAGTGAGTGTCAGTGAGGACAGGCCTGTGGGCACCTCCATTGCTACCCTCAGTGCCAA
CGATGAGGACACAGGAGAGAATGCCCGCATCACCTACGTGATTCAGGACCCCGTGCCGCAGTTCCGCA
TTGACCCCGACAGTGGCACCATGTACACCATGATGGAGCTGGACTATGAGAACCAGGTCGCCTACACG
CTGACCATCATGGCCCAGGACAACGGCATCCCGCAGAAATCAGACACCACCACCCTAGAGATCCTCAT
CCTCGATGCCAATGACAATGCACCCCAGTTCCTGTGGGATTTCTACCAGGGTTCCATCTTTGAGGATG
CTCCACCCTCGACCAGCATCCTCCAGGTCTCTGCCACGGACCGGGACTCAGGTCCCAATGGGCGTCTG
CTGTACACCTTCCAGGGTGGGGACGACGGCGATGGGGACTTCTACATCGAGCCCACGTCCGGTGTGAT
TCGCACCCAGCGCCGGCTGGACCGGGAGAATGTGGCCGTGTACAACCTTTGGGCTCTGGCTGTGGATC
GGGGCAGTCCCACTCCCCTTAGCGCCTCGGTAGAAATCCAGGTGACCATCTTGGACATTAATGACAAT
GCCCCCATGTTTGAGAAGGACGAACTGGAGCTGTTTGTTGAGGAGAACAACCCAGTGGGGTCGGTGGT
GGCAAAGATTCGTGCTAACGACCCTGATGAAGGCCCTAATGCCCAGATCATGTATCAGATTGTGGAAG
GGGACATGCGGCATTTCTTCCAGCTGGACCTGCTCAACGGGGACCTGCGTGCCATGGTGGAGCTGGAC
TTTGAGGTCCGGCGGGAGTATGTGCTGGTGGTGCAGGCCACGTCGGCTCCGCTGGTGAGCCGAGCCAC
GGTGCACATCCTTCTCGTGCTCGAG

| | | | |
|---|---|---|---|
| NOV21d, 317460050 | SEQ ID NO: 282 | 847 aa | MW at 93611.5kD |
| Protein Sequence | | | |

KLYQVALFENEPAGTLILQLHAHHTIEGEEERVSYYMEGLFDERSRGYFRIDSAAGAVSTDSVLDRET
KETHVLRVKAVDYSTPPRSATTYITVLVKDTNDHSPVFEQSEYRERVRENLEVGYEVLTIRASDRDSP
INANLRYRVLGGAWDVFQLNESSGVVSTRAVLDREEAAEYQLLVEANDQGRNPGPLSATATVYIEVED
ENDNYPQFSEQNYVVQVPEDVGLNTAVLRVQATDRDQGQNAAIHYSILSGNVAGQFYLHSLSGILDVI
NPLDFEDVQKYSLSIKAQDGGRPPLINSSGVVSVQVLDVNDNEPIFVSSPFQATVLENVPLGYPVVHI
QAVDADSGENARLHYRLVDTASTFLGGGSAGPKNPAPTPDFPFQIRNSSGWITVCAELDREEVEHYSF
GVEAVDHGWPPMSSSTSVSITVLDVNDNDPVFTQPTYELRLNEDAAVGSSVLTLQARDRDANSVITYQ
LTGGNTRNRFALSSQRGGGLITLALPLDYKQEQQYVLAVTASDGTRSHTAHVLINVTDANTHRPVFQS
SHYTVSVSEDRPVGTSIATLSANDEDTGENARITYVIQDPVPQFRIDPDSGTMYTMMELDYENQVAYT
LTIMAQDNGIPQKSDTTTLEILILDANDNAPQFLWDFYQGSIFEDAPPSTSILQVSATDRDSGPNGRL
LYTFQGGDDGDGDFYIEPTSGVIRTQRRLDRENVAVYNLWALAVDRGSPTPLSASVEIQVTILDINDN
APMFEKDELELFVEENNPVGSVVAKIRANDPDEGPNAQIMYQIVEGDMRHFFQLDLLNGDLRAMVELD
FEVRREYVLVVQATSAPLVSRATVHILLVLE

| | | |
|---|---|---|
| NOV21e, SNP13382483 of | SEQ ID NO: 283 | 9087 bp |
| CG51965-01, DNA Sequence | ORF Start: ATG at 1 | ORF Stop: TGA at 9085 |
| | SNP Pos: 8435 | SNP Change: G to C |

ATGGCGCCGCCGCCGCCGCCCGTGCTGCCCGTGCTGCTGCTCCTGGCCGCCGCCGCCGCCCTGCCGGC
GATGGGGCTGCGAGCGGCCGCCTGGGAGCCGCGCGTACCCGGCGGGACCCGCGCCTTCGCCCTCCGGC
CCGGCTGTACCTACGCGGTGGGCGCCGCTTGCACGCCCCGGGCGCCGCGGGAGCTGCTGGACGTGGGC
CGCGATGGGCGGCTGGCAGGACGTCGGCGCGTCTCGGGCGCGGGGCGCCCGCTGCCGCTGCAAGTCCG
CTTGGTGGCCCGCAGTGCCCCGACGGCGCTGAGCCGCCGCCTGCGGGCGCGCACGCACCTTCCCGGCT
GCGGAGCCCGTGCCCGGCTCTGCGGAACCGGTGCCCGGCTCTGCGGGGCGCTCTGCTTCCCCGTCCCC
GGCGGCTGCGCGGCCGCGCAGCATTCGGCGCTCGCAGCTCCGACCACCTTACCCGCCTGCCGCTGCCC
GCCGCGCCCCAGGCCCCGCTGTCCCGGCCGTCCCATCTGCCTGCCGCCGGGCGGCTCGGTCCGCCTGC
GTCTGCTGTGCGCCCTGCGGCGCGCGGCTGGCGCCGTCCGGGTGGGACTGGCGCTGGAGGCCGCCACC
GCGGGGACGCCCTCCGCGTCGCCATCCCCATCGCCGCCCCTGCCGCCGAACTTGCCCGAAGCCCGGGC
GGGGCCGGCGCGACGGGCCCGGCGGGGCACGAGCGGCAGAGGGAGCCTGAAGTTTCCGATGCCCAACT
ACCAGGTGGCGTTGTTTGAGAACGAACCGGCGGGCACCCTCATCCTCCAGCTGCACGCGCACTACACC
ATCGAGGGCGAGGAGGAGCGCGTGAGCTATTACATGGAGGGGCTGTTCGACGAGCGCTCCCGGGGCTA
CTTCCGAATCGACTCTGCCACGGGCGCCGTGAGCACGGACAGCGTACTGGACCGCGAGACCAAGGAGA
CGCACGTCCTCAGGGTGAAAGCCGTGGACTACAGTACGCCGCCGCGCTCGGCCACCACCTACATCACT
GTCTTGGTCAAAGACACCAACGACCACAGCCCGGTCTTCGAGCAGTCGGAGTACCGCGAGCGCGTGCG
GGAGAACCTGGAGGTGGGCTACGAGGTGCTGACCATCCGCGCCAGCGACCGCGACTCGCCCATCAACG
CCAACTTGCGTTACCGCGTGTTGGGGGGCGCGTGGGACGTCTTCCAGCTCAACGAGAGCTCTGGCGTG
GTGAGCACACGGGCGGTGCTGGACCGGGAGGAGGCGGCCGAGTACCAGCTCCTGGTGGAGGCCAACGA
CCAGGGGCGCAATCCGGGCCCGCTCAGTGCCACGGCCACCGTGTACATCGAGGTGGAGGACGAGAACG
ACAACTACCCCCAGTTCAGCGAGCAGAACTACGTGGTCCAGGTGCCCGAGGACGTGGGGCTCAACACG
GCTGTGCTGCGAGTGCAGGCCACGGACCGGGACCAGGGCCAGAACGCGGCCATTCACTACAGCATCCT
CAGCGGGAACGTGGCCGGCCAGTTCTACCTGCACTCGCTGAGCGGGATCCTGGATGTGATCAACCCCT
TGGATTTCGAGGATGTCCAGAAATACTCGCTGAGCATTAAGGCCCAGGATGGGGGCCGGCCCCCGCTC
ATCAATTCTTCAGGGGTGGTGTCTGTGCAGGTGCTGGATGTCAACGACAACGAGCCTATCTTTGTGAG
CAGCCCCTTCCAGGCCACGGTGCTGGAGAATGTGCCCCTGGGCTACCCCGTGGTGCACATTCAGGCGG
TGGACGCGGACTCTGGAGAGAACGCCCGGCTGCACTATCGCCTGGTGGACACGGCCTCCACCTTTCTG
GGGGGCGGCAGCGCTGGGCCTAAGAATCCTGCCCCCACCCCTGACTTCCCCTTCCAGATCCACAACAG
CTCCGGTTGGATCACAGTGTGTGCCGAGCTGGACCGCGAGGAGGTGGAGCACTACAGCTTCGGGGTGG
AGGCGGTGGACCACGGCTCGCCCCCCATGAGCTCCTCCACCAGCGTGTCCATCACGGTGCTGGACGTG
AATGACAACGACCCGGTGTTCACGCAGCCCACCTACGAGCTTCGTCTGAATGAGGATGCGGCCGTGGG
GAGCAGCGTGCTGACCCTGCAGGCCCGCGACCGTGACGCCAACAGTGTGATTACCTACCAGCTCACAG
GCGGCAACACCCGGAACCGCTTTGCACTCAGCAGCCAGAGAGGGGGCGGCCTCATCACCCTGGCGCTA
CCTCTGGACTACAAGCAGGAGCAGCAGTACGTGCTGGCGGTGACAGCATCCGACGGCACACGGTCGCA
CACTGCGCATGTCCTAATCAACGTCACTGATGCCAACACCCACAGGCCTGTCTTTCAGAGCTCCCATT
ACACAGTGAGTGTCAGTGAGGACAGGCCTGTGGGCACCTCCATTGCTACCCTCAGTGCCAACGATGAG
GACACAGGAGAGAATGCCCGCATCACCTACGTGATTCAGGACCCCGTGCCGCAGTTCCGCATTGACCC
CGACAGTGGCACCATGTACACCATGATGGAGCTGGACTATGAGAACCAGGTCGCCTACACGCTGACCA
TCATGGCCCAGGACAACGGCATCCCGCAGAAATCAGAAACCACCACCCTAGAGATCCTCATCCTCGAT
GCCAATGACAATGCACCCCAGTTCCTGTGGGATTTCTACCAGGGTTCCATCTTTGAGGATGCTCCACC
CTCGACCAGCATCCTCCAGGTCTCTGCCACGGACCGGGACTCAGGTCCCAATGGGCGTCTGCTGTACA
CCTTCCAGGGTGGGGACGACGGCGATGGGGACTTCTACATCGAGCCCACGTCCGGTGTGATTCGCACC
CAGCGCCGGCTGGACCGGGAGAATGTGGCCGTGTACAACCTTTGGGCTCTGGCTGTGGATCGGGGCAG
TCCCACTCCCCTTAGCGCCTCGGTAGAAATCCAGGTGACCATCTTGGACATTAATGACAATGCCCCCA
TGTTTGAGAAGGACGAACTGGAGCTGTTTGTTGAGGAGAACAACCCAGTGGGGTCGGTGGTGGCAAAG
ATTCGTGCTAACGACCCTGATGAAGGCCCTAATGCCCAGATCATGTATCAGATTGTGGAAGGGGACAT
GCGGCATTTCTTCCAGCTGGACCTGCTCAACGGGGACCTGCGTGCCATGGTGGAGCTGGACTTTGAGG
TCCGGCGGGAGTATGTGCTGGTGGTGCAGGCCACGTCGGCTCCGCTGGTGAGCCGAGCCACGGTGCAC
ATCCTTCTCGTGGACCAGAATGACAACCCGCCTGTGCTGCCCGACTTCCAGATCCTCTTCAACAACTA
TGTCACCAACAAGTCCAACAGTTTCCCCACCGGCGTGATCGGCTGCATCCCGGCCCATGACCCCGACG
TGTCAGACAGCCTCAACTACACCTTCGTGCAGGGCAACGAGCTGCGCCTGTTGCTGCTGGACCCCGCC
ACGGGCGAACTGCAGCTCAGCCGCGACCTGGACAACAACCGGCCGCTGGAGGCGCTCATGGAGGTGTC
TGTGTCTGCAGATGGCATCCACAGCGTCACGGCCTTCTGCACCCTGCGTGTCACCATCATCACGGACG
ACATGCTGACCAACAGCATCACTGTCCGCCTGGAGAACATGTCCCAGGAGAAGTTCCTGTCCCCGCTG
CTGGCCCTCTTCGTGGAGGGGGTGGCCGCCGTGCTGTCCACCACCAAGGACGACGTCTTCGTCTTCAA
CGTCCAGAACGACACCGACGTCAGCTCCAACATCCTGAACGTGACCTTCTCGGCGCTGCTGCCTGGCG
GCGTCCGCGGCCAGTTCTTCCCGTCGGAGGACCTGCAGGAGCAGATCTACCTGAATCGGACGCTGCTG
ACCACCATCTCCACGCAGCGCGTGCTGCCCTTCGACGACAACATCTGCCTGCGCGAGCCCTGCGAGAA
CTACATGAAGTGCGTGTCCGTTCTGCGATTCGACAGCTCCGCGCCCTTCCTCAGCTCCACCACCGTGC
TCTTCCGGCCCATCCACCCCATCAACGGCCTGCGCTGCCGCTGCCCGCCCGGCTTCACCGGCGACTAC
TGCGAGACGGAGATCGACCTCTGCTACTCCGACCCGTGCGGCGCCAACGGCCGCTGCCGCAGCCGCGA
GGGCGGCTACACCTGCGAGTGCTTCGAGGACTTCACTGGAGAGCACTGTGAGGTGGATGCCCGCTCAG
GCCGCTGTGCCAACGGGGTGTGCAAGAACGGGGGCACCTGCGTGAACCTGCTCATCGGCGGCTTCCAC
TGCGTGTGTCCTCCTGGCGAGTATGAGAGGCCCTACTGTGAGGTGACCACCAGGAGCTTCCCGCCCCA
GTCCTTCGTCACCTTCCGGGGCCTGAGACAGCGCTTCCACTTCACCATCTCCCTCACGTTTGCCACTC
AGGAAAGGAACGGCTTGCTTCTCTACAACGGCCGCTTCAATGAGAAGCACGACTTCATCGCCCTGGAG
ATCGTGGACGAGCAGGTGCAGCTCACCTTCTCTGCAGGTGCAGGCGAGACAACAACGACCGTGGCACC
GAAGGTTCCCAGTGGTGTGAGTGACGGGCGGTGGCACTCTGTGCAGGTGCAGTACTACAACAAGGTAA
GATGGGCCCCACCACTTCCCCCTGGCCCCCAGCCCAATATTGGCCACCTGGGCCTGCCCCATGGGCCG
TCCGGGGAAAAGATGGCCGTGGTGACAGTGGATGATTGTGACACAACCATGGCTGTGCGCTTTGGAAA
GGACATCGGGAACTACAGCTGCGCTGCCCAGGGCACTCAGACCGGCTCCAAGAAGTCCCTGGATCTGA
CCGGCCCTCTACTCCTGGGGGGTGTCCCCGACCTGCCAGAAGACTTCCCAGTGCACAACCGGCAGTTC
GTGGGCTGCATGCGGAACCTGTCAGTCGACGGCAAAAATGTGGACATGGCCGGATTCATCGCCAACAA
TGGCACCCGGGAAGGCTGCGCTGCTCGGAGGAACTTCTGCGATGGGAGGCGGTGTCAGAATGGAGGCA
CCTGTGTCAACAGGTGGAATATGTATCTGTGTGAGTGTCCACTCCGATTCGGCGGGAAGAACTGTGAG
CAAGCCATGCCTCACCCCCAGCTCTTCAGCGGTGAGAGCGTCGTGTCCTGGAGTGACCTGAACATCAT
CATCTCTGTGCCCTGGTACCTGGGGCTCATGTTCCGGACCCGGAAGGAGGACAGCGTTCTGATGGAGG
CCACCAGTGGTGGGCCCACCAGCTTTCGCCTCCAGATCCTGAACAACTACCTCCAGTTTGAGGTGTCC
CACGGCCCCTCCGATGTGGAGTCCGTGATGCTGTCCGGGTTGCGGGTGACCGACGGGGAGTGGCACCA
CCTGCTGATCGAGCTGAAGAATGTTAAGGAGGACAGTGAGATGAAGCACCTGGTCACCATGACCTTGG
ACTATGGGATGGACCAGAACAAGGCAGATATCGGGGGCATGCTTCCCGGGCTGACGGTAAGGAGCGTG
GTGGTCGGAGGCGCCTCTGAAGACAAGGTCTCCGTGCGCCGTGGATTCCGAGGCTGCATGCAGGGAGT
GAGGATGGGGGGGACGCCCACCAACGTCGCCACCCTGAACATGAACAACGCACTCAAGGTCAGGGTGA
AGGACGGCTGTGATGTGGACGACCCCTGTACCTCGAGCCCCTGTCCCCCCAATAGCCGCTGCCACGAC
GCCTGGGAGGACTACAGCTGCGTCTGTGACAAAGGGTACCTTGGAATAAACTGTGTGGATGCCTGTCA
CCTGAACCCCTGCGAGAACATGGGGGCCTGCGTGCGCTCCCCCGGCTCCCCGCAGGGCTACGTGTGCG
AGTGTGGGCCCAGTCACTACGGGCCGTACTGTGAGAACAAACTCGACCTTCCGTGCCCCAGAGGCTGG
TGGGGGAACCCCGTCTGTGGACCCTGCCACTGTGCCGTCAGCAAAGGCTTTGATCCCGACTGTAATAA
GACCAACGGCCAGTGCCAATGCAAGGAGAATTACTACAAGCTCCTAGCCCAGGACACCTGTCTGCCCT
GCGACTGCTTCCCCCATGGCTCCCACAGCCGCACTTGCGACATGGCCACCGGGCAGTGTGCCTGCAAG
CCCGGCGTCATCGGCCGCCAGTGCAACCGCTGCGACAACCCGTTTGCCGAGGTCACCACGCTCGGCTG
TGAAGTGATCTACAATGGCTGTCCCAAAGCATTTGAGGCCGGCATCTGGTGGCCACAGACCAAGTTCG
GGCAGCCGGCTGCGGTGCCATGCCCTAAGGGATCCGTTGGAAATGCGGTCCGACACTGCAGCGGGGAG
AAGGGCTGGCTGCCCCCAGAGCTCTTTAACTGTACCACCATCTCCTTCGTGGACCTCAGGGCCATGAA
TGAGAAGCTGAGCCGCAATGAGACGCAGGTGGACGGCGCCAGGGCCCTGCAGCTGGTGAGGGCGCTGC
GCAGTGCTACACAGCACACGGGCACGCTCTTTGGCAATGACGTGCGCACGGCCTACCAGCTGCTGGGC
CACGTCCTTCAGCACGAGAGCTGGCAGCAGGGCTTCGACCTGGCAGCCACGCAGGACGCCGACTTTCA
CGAGGACGTCATCCACTCGGGCAGCGCCCTCCTGGCCCCAGCCACCAGGGCGGCGTGGGAGCAGATCC
AGCGGAGCGAGGGCGGCACGGCACAGCTGCTCCGGCGCCTCGAGGGCTACTTCAGCAACGTGGCACGC
AACGTGCGGCGGACGTACCTGCGGCCCTTCGTCATCGTCACCGCCAACATGGTTCTTGCTGTCGACAT
CTTTGACAAGTTCAACTTTACGGGAGCCAGGGTCCCGCGATTCGACACCATCCATGAAGAGTTCCCCA
GGGAGCTGGAGTCCTCCGTCTCCTTCCCAGCCGACTTCTTCAGACCACCTGAAGAAAAAGAAGGCCCC
CTGCTGAGGCCGGCTGGCCGGAGGACCACCCCGCAGACCACGCGCCCGGGGCCTGGGGCCGAGAGGGA
GGCCCCGATCAGCAGGCGGAGGCGACACCCTGATGACGCTGGCCAGTTCGCCGTCGCTCTGGTCATCA
TTTACCGCACCCTGGGGCAGCTCCTGCCCGAGCGCTACGACCCCGACCGTCGCAGCCTCCGGTTGCCT
CACCGGCCCATCATTAATACCCCGATGGTGAGCACGCTGGTGTACAGCGAGGGGGCTCCGCTCCCGAG
ACCCCTGGAGAGGCCCGTCCTGGTGGAGTTCGCCCTGCTGGAGGTGGAGGAGCGAACCAAGCCTGTCT
GCGTGTTCTGGAACCACTCCCTGGCCGTTGGTGGGACGGGAGGGTGGTCTGCCCGGGGCTGCGAGCTC
CTGTCCAGGAACCGGACACATGTCGCCTGCCAGTGCAGCCACACAGCCAGCTTTGCGGTGCTCATGGA
TATCTCCAGGCGTGAGAACGGGGAGGTCCTGCCTCTGAAGATTGTCACCTATGCCGCTGTGTCCTTGT
CACTGGCAGCCCTGCTGGTGGCCTTCGTCCTCCTGAGCCTGGTCCGCATGCTGCGCTCCAACCTGCAC
AGCATTCACAAGCACCTCGCCGTGGCGCTCTTCCTCTCTCAGCTGGTGTTCGTGATTGGGATCAACCA
GACGGAAAACCCGTTTCTGTGCACAGTGGTTGCCATCCTCCTCCACTACATCTACATGAGCACCTTTG
CCTGGACCCTCGTGGAGAGCCTGCATGTCTACCGCATGCTGACCGAGGTGCGCAACATCGACACGGGG
CCCATGCGGTTCTACTACGTCGTGGGCTGGGGCATCCCGGCCATTGTCACAGGACTGGCGGTCGGCCT
GGACCCCCAGGGCTACGGGAACCCCGACTTCTGCTGGCTGTCGCTTCAAGACACCCTGATTTGGAGCT
TTGCGGGGCCCATCGGAGCTGTTATAATCATCAACACAGTCACTTCTGTCCTATCTGCAAAGGTTTCC
TGCCAAAGAAAGCACCATTATTATGGGAAAAAAGGGATCGTCTCCCTGCTGAGGACCGCATTCCTCCT
GCTGCTGCTCATCAGCGCCACCTGGCTGCTGGGGCTGCTGGCTGTGAACCGCGATGCACTGAGCTTTC
ACTACCTCTTCGCCATCTTCAGCGGCTTACAGGGCCCCTTCGTCCTCCTTTTCCACTGCGTGCTCAAC
CAGGAGGTCCGGAAGCACCTGAAGGGCGTGCTCGGCGGGAGGAAGCTGCACCTGGAGGACTCCGCCAC
CACCAGGGCCACCCTGCTGACGCGCTCCCTCAACTGCAACACCACCTTCGGTGACGGGCCTGACATGC
TGCGCACAGACTTGGGCGAGTCCACCGCCTCGCTGGACAGCATCGTCAGGGATGAAGGGATCCAGAAG
CTCGGCGTGTCCTCTGGGCTGGTGAGGGGCAGCCACGGAGAGCCAGACGCGTCCCTCATGCCCAGGAG
CTCCAAGGATCCCCCTGGCCACGATTCCGACTCAGATAGCGAGCTGTCCCTGGATGAGCAGAGCAGCT
CTTACGCCTCCTCACACTCGTCAGACAGCGAGGACGATGGGGTGGGAGCTGAGGAAAAATGGGACCCG
GCCAGGGGCGCCGTCCACAGCACCCCCAAAGGGGACGCTGTGGCCAACCACGTTCCGGCCGGCTGGCC
CGACCAGAGCCTGGCTGAGAGTGACAGTGAGGACCCCAGCGGCAAGCCCCGCCTGAAGGTGGAGACCA
AGGTCAGCGTGGAGCTGCACCGCGAGGAGCAGGGCAGTCACCGTGGAGAGTACCCCCCCGGACAGGAG
AGCGGGGGCGCAGCCAGGCTTGCTAGCAGCCAGCCCCCAGAGCAGAGGAGCATCTTGAAAAATAAAGT
CACCTACCCGCCGCCGCTGACGCTGACGGAGCAGACGCTGAAGGGCCGGCTCCGGGAGAAGCTGGCCG
ACTGTGAGCAGAGCCCCACATCCTCGCGCACGTCTTCCCTGGGCTCTGGCGGCCCCGACTGCGCCATC
ACAGTCAAGAGCCCTGGGAGGGAGCCGGGGCGTGACCACCTCAACGGGGTGGCCATGAATGTGCGCAC
TGGGAGCGCCCAGGCCGATGGCTCCGACTCTGAGAAACCGTGA

| | | | |
|---|---|---|---|
| NOV21e, SNP13382483 of | SEQ ID NO: 284 | 3028 aa | MW at 330849.8kD |
| CG51965-01, Protein | SNP Pos: 2812 | | SNP Change: Cys to Ser |
| Sequence | | | |

MAPPPPPVLPVLLLLAAAAALPAMGLRAAAWEPRVPGGTRAFALRPGCTYAVGAACTPRAPRELLDVG
RDGRLAGRRRVSGAGRPLPLQVRLVARSAPTALSRRLRARTHLPGCGARARLCGTGARLCGALCFPVP
GGCAAAQHSALAAPTTLPACRCPPRPRPRCPGRPICLPPGGSVRLRLLCALRRAAGAVRVGLALEAAT
AGTPSASPSPSPPLPPNLPEARAGPARRARRGTSGRGSLKFPMPNYQVALFENEPAGTLILQLHAHYT
IEGEEERVSYYMEGLFDERSRGYFRIDSATGAVSTDSVLDRETKETHVLRVKAVDYSTPPRSATTYIT
VLVKDTNDHSPVFEQSEYRERVRENLEVGYEVLTIRASDRDSPINANLRYRVLGGAWDVFQLNESSGV
VSTRAVLDREEAAEYQLLVEANDQGRNPGPLSATATVYIEVEDENDNYPQFSEQNYVVQVPEDVGLNT
AVLRVQATDRDQGQNAAIHYSILSGNVAGQFYLHSLSGILDVINPLDFEDVQKYSLSIKAQDGGRPPL
INSSGVVSVQVLDVNDNEPIFVSSPFQATVLENVPLGYPVVHIQAVDADSGENARLHYRLVDTASTFL
GGGSAGPKNPAPTPDFPFQIHNSSGWITVCAELDREEVEHYSFGVEAVDHGSPPMSSSTSVSITVLDV
NDNDPVFTQPTYELRLNEDAAVGSSVLTLQARDRDANSVITYQLTGGNTRNRFALSSQRGGGLITLAL
PLDYKQEQQYVLAVTASDGTRSHTAHVLINVTDANTNRPVFQSSHYTVSVSEDRPVGTSIATLSANDE
DTGENARITYVIQDPVPQFRIDPDSGTMYTMMELDYENQVAYTLTIMAQDNGIPQKSDTTTLEILILD
ANDNAPQFLWDFYQGSIFEDAPPSTSILQVSATDRDSGPNGRLLYTFQGGDDGDGDFYIEPTSGVIRT
QRRLDRENVAVYNLWAIAVDRGSPTPLSASVEIQVTILDINDNAPMFEKDELELFVEENNPVGSVVAK
IRANDPDEGPNAQIMYQIVEGDMRHFFQLDLLNGDLRAMVELDFEVRREYVLVVQATSAPLVSRATVH
ILLVDQNDNPPVLPDFQILFNNYVTNKSNSFPTGVIGCIPAHDPDVSDSLNYTFVQGNELRLLLLDPA
TGELQLSRDLDNNRPLEALMEVSVSADGIHSVTAFCTLRVTIITDDMLTNSITVRLENMSQEKFLSPL
LALFVEGVAAVLSTTKDDVFVFNVQNDTDVSSNILNVTFSALLPGGVRGQFFPSEDLQEQIYLNRTLL
TTISTQRVLPFDDNICLREPCENYMKCVSVLRFDSSAPFLSSTTVLFRPIHPINGLRCRCPPGFTGDY
CETEIDLCYSDPCGANGRCRSREGGYTCECFEDFTGEHCEVDARSGRCANGVCKNGGTCVNLLIGGFH
CVCPPGEYERPYCEVTTRSFPPQSFVTFRGLRQRFHFTISLTFATQERNGLLLYNGRFNEKHDFIALE
IVDEQVQLTFSAGAGETTTTVAPKVPSGVSDGRWHSVQVQYYNKVRWAPPLPPGPQPNIGHLGLPHGP
SGEKMAVVTVDDCDTTMAVRFGKDIGNYSCAAQGTQTGSKKSLDLTGPLLLGGVPNLPEDFPVHNRQF
VGCMRNLSVDGKNVDMAGFIANNGTREGCAARRNFCDGRRCQNGGTCVNRWNMYLCECPLRFGGKNCE
QANPHPQLFSGESVVSWSDLNIIISVPWYLGLMFRTRKEDSVLMEATSGGPTSFRLQILNNYLQFEVS
HGPSDVESVMLSGLRVTDGEWHHLLIELKNVKEDSEMKHLVTMTLDYGMDQNKADIGGMLPGLTVRSV
VVGGASEDKVSVRRGFRGCMQGVRMGGTPTNVATLNMNNALKVRVKDGCDVDDPCTSSPCPPNSRCHD
AWEDYSCVCDKGYLGINCVDACHLNPCENMGACVRSPGSPQGYVCECGPSHYGPYCENKLDLPCPRGW
WGNPVCGPCHCAVSKGFDPDCNKTNGQCQCKENYYKLLAQDTCLPCDCFPHGSHSRTCDMATGQCACK
PGVIGRQCNRCDNPFAEVTTLGCEVIYNGCPKAFEAGIWWPQTKFGQPAAVPCPKGSVGNAVRHCSGE
KGWLPPELFNCTTISFVDLRAMNEKLSRNETQVDGARALQLVRALRSATQHTGTLFGNDVRTAYQLLG
HVLQHESWQQGFDLAATQDADFHEDVIHSGSALLAPATRAAWEQIQRSEGGTAQLLRRLEGYFSNVAR
NVRRTYLRPFVIVTANMVLAVDIFDKFNFTGARVPRFDTIHEEFPRELESSVSFPADFFRPPEEKEGP
LLRPAGRRTTPQTTRPGPGTEREAPISRRRRHPDDAGQFAVALVIIYRTLGOLLPERYDPDRRSLRLP
HRPIINTPMVSTLVYSEGAPLPRPLERPVLVEFALLEVEERTKPVCVFWNHSLAVGGTGGWSARGCEL
LSRNRTHVACQCSHTASFAVLMDISRRENGEVLPLKIVTYAAVSLSLAALLVAFVLLSLVRMLRSNLH
SIHKHLAVALFLSQLVFVIGINQTENPFLCTVVAILLHYIYMSTFAWTLVESLHVYRMLTEVRNIDTG
PMRFYYVVGWGIPAIVTGLAVGLDPQGYGNPDFCWLSLQDTLIWSFAGPIGAVIIINTVTSVLSAKVS
CQRKHHYYGKKGIVSLLRTAFLLLLLISATWLLGLLAVNRDALSFHYLFAIFSGLQGPFVLLFHCVLN
QEVRKHLKGVLGGRKLHLEDSATTRATLLTRSLNCNTTFGDGPDMLRTDLGESTASLDSIVRDEGIQK
LGVSSGLVRGSHGEPDASLMPRSSKDPPGHDSDSDSELSLDEOSSSYASSHSSDSEDDGVGAEEKWDP
ARGAVHSTPKGDAVANHVPAGWPDQSLAESDSEDPSGKPRLKVETKVSVELHREEQGSHRGEYPPDQE
SGGAARLASSQPPEQRSILKNKVTYPPPLTLTEQTLKGRLREKLADCEQSPTSSRTSSLGSGGPDCAI
TVKSPGREPGRDHLNGVANNVRTGSAQADGSDSEKP

| | | |
|---|---|---|
| NOV21f, SNP13382484 of | SEQ ID NO: 285 | 9087 bp |
| CG51965-01, DNA Sequence | ORF Start: ATG at 1 | ORF Stop: TGA at 9085 |
| | SNP Pos: 8592 | SNP Change: C to A |

ATGGCGCCGCCGCCGCCGCCCGTGCTGCCCGTGCTGCTGCTCCTGGCCGCCGCCGCCGCCCTGCCGGC
GATGGGGCTGCGAGCGGCCGCCTGGGAGCCGCGCGTACCCGGCGGGACCCGCGCCTTCGCCCTCCGGC
CCGGCTGTACCTACGCGGTGGGCGCCGCTTGCACGCCCCGGGCGCCGCGGGAGCTGCTGGACGTGGGC
CGCGATGGGCGGCTGGCAGGACGTCGGCGCGTCTCGGGCGCGGGGCGCCCGCTGCCGCTGCAAGTCCG
CTTGGTGGCCCGCAGTGCCCCGACGGCGCTGAGCCGCCGCCTGCGGGCGCGCACGCACCTTCCCGGCT
GCGGAGCCCGTGCCCGGCTCTGCGGAACCGGTGCCCGGCTCTGCGGGGCGCTCTGCTTCCCCGTCCCC
GGCGGCTGCGCGGCCGCGCAGCATTCGGCGCTCGCAGCTCCGACCACCTTACCCGCCTGCCGCTGCCC
GCCGCGCCCCAGGCCCCGCTGTCCCGGCCGTCCCATCTGCCTGCCGCCGGGCGGCTCGGTCCGCCTGC
GTCTGCTGTGCGCCCTGCGGCGCGCGGCTGGCGCCGTCCGGGTGGGACTGGCGCTGGAGGCCGCCACC
GCGGGGACGCCCTCCGCGTCGCCATCCCCATCGCCGCCCCTGCCGCCGAACTTGCCCGAAGCCCGGGC
GGGGCCGGCGCGACGGGCCCGGCGGGGCACGAGCGGCAGAGGGAGCCTGAAGTTTCCGATGCCCAACT
ACCAGGTGGCGTTGTTTGAGAACGAACCGGCGGGCACCCTCATCCTCCAGCTGCACGCGCACTACACC
ATCGAGGGCGAGGAGGAGCGCGTGAGCTATTACATGGAGGGGCTGTTCGACGAGCGCTCCCGGGGCTA
CTTCCGAATCGACTCTGCCACGGGCGCCGTGAGCACGGACAGCGTACTGGACCGCGAGACCAAGGAGA
CGCACGTCCTCAGGGTGAAAGCCGTGGACTACAGTACGCCGCCGCGCTCGGCCACCACCTACATCACT
GTCTTGGTCAAAGACACCAACGACCACAGCCCGGTCTTCGAGCAGTCGGAGTACCGCGAGCGCGTGCG
GGAGAACCTGGAGGTGGGCTACGAGGTGCTGACCATCCGCGCCAGCGACCGCGACTCGCCCATCAACG
CCAACTTGCGTTACCGCGTGTTGGGGGGCGCGTGGGACGTCTTCCAGCTCAACGAGAGCTCTGGCGTG
GTGAGCACACGGGCGGTGCTGGACCGGGAGGAGGCGGCCGAGTACCAGCTCCTGGTGGAGGCCAACGA
CCAGGGGCGCAATCCGGGCCCGCTCAGTGCCACGGCCACCGTGTACATCGAGGTGGAGGACGAGAACG
ACAACTACCCCCAGTTCAGCGAGCAGAACTACGTGGTCCAGGTGCCCGAGGACGTGGGGCTCAACACG
GCTGTGCTGCGAGTGCAGGCCACGGACCGGGACCAGGGCCAGAACGCGGCCATTCACTACAGCATCCT
CAGCGGGAACGTGGCCGGCCAGTTCTACCTGCACTCGCTGAGCGGGATCCTGGATGTGATCAACCCCT
TGGATTTCGAGGATGTCCAGAAATACTCGCTGAGCATTAAGGCCCAGGATGGGGGCCGGCCCCCGCTC
ATCAATTCTTCAGGGGTGGTGTCTGTGCAGGTGCTGGATGTCAACGACAACGAGCCTATCTTTGTGAG
CAGCCCCTTCCAGGCCACGGTGCTGGAGAATGTGCCCCTGGGCTACCCCGTGGTGCACATTCAGGCGG
TGGACGCGGACTCTGGAGAGAACGCCCGGCTGCACTATCGCCTGGTGGACACGGCCTCCACCTTTCTG
GGGGGCGGCAGCGCTGGGCCTAAGAATCCTGCCCCCACCCCTGACTTCCCCTTCCAGATCCACAACAG
CTCCGGTTGGATCACAGTGTGTGCCGAGCTGGACCGCGAGGAGGTGGAGCACTACAGCTTCGGGGTGG
AGGCGGTGGACCACGGCTCGCCCCCCATGAGCTCCTCCACACGCGTGTCCATCACGGTGCTGGACGTG
AATGACAACGACCCGGTGTTCACGCAGCCCACCTACGAGCTTCGTCTGAATGAGGATGCGGCCGTGGG
GAGCAGCGTGCTGACCCTGCAGGCCCGCGACCGTGACGCCAACAGTGTGATTACCTACCAGCTCACAG
GCGGCAACACCCGGAACCGCTTTGCACTCAGCAGCCAGAGAGGGGGCGGCCTCATCACCCTGGCGCTA
CCTCTGGACTACAAGCAGGAGCAGCAGTACGTGCTGGCGGTGACAGCATCCGACGGCACACGGTCGCA
CACTGCGCATGTCCTAATCAACGTCACTGATGCCAACACCCACAGGCCTGTCTTTCAGAGCTCCCATT
ACACAGTGAGTGTCAGTGAGGACAGGCCTGTGGGCACCTCCATTGCTACCCTCAGTGCCAACGATGAG
GACACAGGAGAGAATGCCCGCATCACCTACGTGATTCAGGACCCCGTGCCGCAGTTCCGCATTGACCC
CGACAGTGGCACCATGTACACCATGATGGAGCTGGACTATGAGAACCAGGTCGCCTACACGCTGACCA
TCATGGCCCAGGACAACGGCATCCCGCAGAAATCAGACACCACCACCCTAGAGATCCTCATCCTCGAT
GCCAATGACAATGCACCCCAGTTCCTGTGGGATTTCTACCAGGGTTCCATCTTTGAGGATGCTCCACC
CTCGACCAGCATCCTCCAGGTCTCTGCCACGGACCGGGACTCAGGTCCCAATGGGCGTCTGCTGTACA
CCTTCCAGGGTGGGGACGACGGCGATGGGGACTTCTACATCGAGCCCACGTCCGGTGTGATTCGCACC
CAGCGCCGGCTGGACCGGGAGAATGTGGCCGTGTACAACCTTTGGGCTCTGGCTGTGGATCGGGGCAG
TCCCACTCCCCTTAGCGCCTCGGTAGAAATCCAGGTGACCATCTTGGACATTAATGACAATGCCCCCA
TGTTTGAGAAGGACGAACTGGAGCTGTTTGTTGAGGAGAACAACCCAGTGGGGTCGGTGGTGGCAAAG
ATTCGTGCTAACGACCCTGATGAAGGCCCTAATGCCCAGATCATGTATCAGATTGTGGAAGGGGACAT
GCGGCATTTCTTCCAGCTGGACCTGCTCAACGGGGACCTGCGTGCCATGGTGGAGCTGGACTTTGAGG
TCCGGCGGGAGTATGTGCTGGTGGTGCAGGCCACGTCGGCTCCGCTGGTGAGCCGAGCCACGGTGCAC
ATCCTTCTCGTGGACCAGAATGACAACCCGCCTGTGCTGCCCGACTTCCAGATCCTCTTCAACAACTA
TGTCACCAACAAGTCCAACAGTTTCCCCACCGGCGTGATCGGCTGCATCCCGGCCCATGACCCCGACG
TGTCAGACAGCCTCAACTACACCTTCGTGCAGGGCAACGAGCTGCGCCTGTTGCTGCTGGACCCCGCC
ACGGGCGAACTGCAGCTCAGCCGCGACCTGGACAACAACCGGCCGCTGGAGGCGCTCATGGAGGTGTC
TGTGTCTGCAGATGGCATCCACAGCGTCACGGCCTTCTGCACCCTGCGTGTCACCATCATCACGGACG
ACATGCTGACCAACAGCATCACTGTCCGCCTGGAGAACATGTCCCAGGAGAAGTTCCTGTCCCCGCTG
CTGGCCCTCTTCGTGGAGGGGGTGGCCGCCGTGCTGTCCACCACCAAGGACGACGTCTTCGTCTTCAA
CGTCCAGAACGACACCGACGTCAGCTCCAACATCCTGAACGTGACCTTCTCGGCGCTGCTGCCTGGCG
GCGTCCGCGGCCAGTTCTTCCCGTCGGAGGACCTGCAGGAGCAGATCTACCTGAATCGGACGCTGCTG
ACCACCATCTCCACGCAGCGCGTGCTGCCCTTCGACGACAACATCTGCCTGCGCGAGCCCTGCGAGAA
CTACATGAAGTGCGTGTCCGTTCTGCGATTCGACAGCTCCGCGCCCTTCCTCAGCTCCACCACCGTGC
TCTTCCGGCCCATCCACCCCATCAACGGCCTGCGCTGCCGCTGCCCGCCCGGCTTCACCGGCGACTAC
TGCGAGACGGAGATCGACCTCTGCTACTCCGACCCGTGCGGCGCCAACGGCCGCTGCCGCAGCCGCGA
GGGCGGCTACACCTGCGAGTGCTTCGAGGACTTCACTGGAGAGCACTGTGAGGTGGATGCCCGCTCAG
GCCGCTGTGCCAACGGGGTGTGCAAGAACGGGGGCACCTGCGTGAACCTGCTCATCGGCGGCTTCCAC
TGCGTGTGTCCTCCTGGCGAGTATGAGAGGCCCTACTGTGAGGTGACCACCAGGAGCTTCCCGCCCCA
GTCCTTCGTCACCTTCCGGGGCCTGAGACAGCGCTTCCACTTCACCATCTCCCTCACGTTTGCCACTC
AGGAAAGGAACGGCTTGCTTCTCTACAACGGCCGCTTCAATGAGAAGCACGACTTCATCGCCCTGGAG
ATCGTGGACGAGCAGGTGCAGCTCACCTTCTCTGCAGGTGCAGGCGAGACAACAACGACCGTGGCACC
GAAGGTTCCCAGTGGTGTGAGTGACGGGCGGTGGCACTCTGTGCAGGTGCAGTACTACAACAAGGTAA
GATGGGCCCCACCACTTCCCCCTGGCCCCCAGCCCAATATTGGCCACCTGGGCCTGCCCCATGGGCCG
TCCGGGGAAAAGATGGCCGTGGTGACAGTGGATGATTGTGACACAACCATGGCTGTGCGCTTTGGAAA
GGACATCGGGAACTACAGCTGCGCTGCCCAGGGCACTCAGACCGGCTCCAAGAAGTCCCTGGATCTGA
CCGGCCCTCTACTCCTGGGGGGTGTCCCCAACCTGCCAGAAGACTTCCCAGTGCACAACCGGCAGTTC
GTGGGCTGCATGCGGAACCTGTCAGTCGACGGCAAAAATGTGGACATGGCCGGATTCATCGCCAACAA
TGGCACCCGGGAAGGCTGCGCTGCTCGGAGGAACTTCTGCGATGGGAGGCGGTGTCAGAATGGAGGCA
CCTGTGTCAACAGGTGGAATATGTATCTGTGTGAGTGTCCACTCCGATTCGGCGGGAAGAACTGTGAG
CAAGCCATGCCTCACCCCCAGCTCTTCAGCGGTGAGAGCGTCGTGTCCTGGAGTGACCTGAACATCAT
CATCTCTGTGCCCTGGTACCTGGGGCTCATGTTCCGGACCCGGAAGGAGGACAGCGTTCTGATGGAGG
CCACCAGTGGTGGGCCCACCAGCTTTCGCCTCCAGATCCTGAACAACTACCTCCAGTTTGAGGTGTCC
CACGGCCCCTCCGATGTGGAGTCCGTGATGCTGTCCGGGTTGCGGGTGACCGACGGGGAGTGGCACCA
CCTGCTGATCGAGCTGAAGAATGTTAAGGAGGACAGTGAGATGAAGCACCTGGTCACCATGACCTTGG
ACTATGGGATGGACCAGAACAAGGCAGATATCGGGGGCATGCTTCCCGGGCTGACGGTAAGGAGCGTG
GTGGTCGGAGGCGCCTCTGAAGACAAGGTCTCCGTGCGCCGTGGATTCCGAGGCTGCATGCAGGGAGT
GAGGATGGGGGGGACGCCCACCAACGTCGCCACCCTGAACATGAACAACGCACTCAAGGTCAGGGTGA
AGGACGGCTGTGATGTGGACGACCCCTGTACCTCGAGCCCCTGTCCCCCCAATAGCCGCTGCCACGAC
GCCTGGGAGGACTACAGCTGCGTCTGTGACAAAGGGTACCTTGGAATAAACTGTGTGGATGCCTGTCA
CCTGAACCCCTGCGAGAACATGGGGGCCTGCGTGCGCTCCCCCGGCTCCCCGCAGGGCTACGTGTGCG
AGTGTGGGCCCAGTCACTACGGGCCGTACTGTGAGAACAAACTCGACCTTCCGTGCCCCAGAGGCTGG
TGGGGGAACCCCGTCTGTGGACCCTGCCACTGTGCCGTCAGCAAAGGCTTTGATCCCGACTGTAATAA
GACCAACGGCCAGTGCCAATGCAAGGAGAATTACTACAAGCTCCTAGCCCAGGACACCTGTCTGCCCT
GCGACTGCTTCCCCCATGGCTCCCACAGCCGCACTTGCGACATGGCCACCGGGCAGTGTGCCTGCAAG
CCCGGCGTCATCGGCCGCCAGTGCAACCGCTGCGACAACCCGTTTGCCGAGGTCACCACGCTCGGCTG
TGAAGTGATCTACAATGGCTGTCCCAAAGCATTTGAGGCCGGCATCTGGTGGCCACAGACCAAGTTCG
GGCAGCCGGCTGCGGTGCCATGCCCTAAGGGATCCGTTGGAAATGCGGTCCGACACTGCAGCGGGGAG
AAGGGCTGGCTGCCCCCAGAGCTCTTTAACTGTACCACCATCTCCTTCGTGGACCTCAGGGCCATGAA
TGAGAAGCTGAGCCGCAATGAGACGCAGGTGGACGGCGCCAGGGCCCTGCAGCTGGTGAGGGCGCTGC
GCAGTGCTACACAGCACACGGGCACGCTCTTTGGCAATGACGTGCGCACGGCCTACCAGCTGCTGGGC
CACGTCCTTCAGCACGAGAGCTGGCAGCAGGGCTTCGACCTGGCAGCCACGCAGGACGCCGACTTTCA
CGAGGACGTCATCCACTCGGGCAGCGCCCTCCTGGCCCCAGCCACCAGGGCGGCGTGGGAGCAGATCC
AGCGGAGCGAGGGCGGCACGGCACAGCTGCTCCGGCGCCTCGAGGGCTACTTCAGCAACGTGGCACGC
AACGTGCGGCGGACGTACCTGCGGCCCTTCGTCATCGTCACCGCCAACATGGTTCTTGCTGTCGACAT
CTTTGACAAGTTCAACTTTACGGGAGCCAGGGTCCCGCGATTCGACACCATCCATGAAGAGTTCCCCA
GGGAGCTGGAGTCCTCCGTCTCCTTCCCAGCCGACTTCTTCAGACCACCTGAAGAAAAAGAAGGCCCC
CTGCTGAGGCCGGCTGGCCGGAGGACCACCCCGCAGACCACGCGCCCGGGGCCTGGCACCGAGAGGGA
GGCCCCGATCAGCAGGCGGAGGCGACACCCTGATGACGCTGGCCAGTTCGCCGTCGCTCTGGTCATCA
TTTACCGCACCCTGGGGCAGCTCCTGCCCGAGCGCTACGACCCCGACCGTCGCAGCCTCCGGTTGCCT
CACCGGCCCATCATTAATACCCCGATGGTGAGCACGCTGGTGTACAGCGAGGGGGCTCCGCTCCCGAG
ACCCCTGGAGAGGCCCGTCCTGGTGGAGTTCGCCCTGCTGGAGGTGGAGGAGCGAACCAAGCCTGTCT
GCGTGTTCTGGAACCACTCCCTGGCCGTTGGTGGGACGGGAGGGTGGTCTGCCCGGGGCTGCGAGCTC
CTGTCCAGGAACCGGACACATGTCGCCTGCCAGTGCAGCCACACAGCCAGCTTTGCGGTGCTCATGGA
TATCTCCAGGCGTGAGAACGGGGAGGTCCTGCCTCTGAAGATTGTCACCTATGCCGCTGTGTCCTTGT
CACTGGCAGCCCTGCTGGTGGCCTTCGTCCTCCTGAGCCTGGTCCGCATGCTGCGCTCCAACCTGCAC
AGCATTCACAAGCACCTCGCCGTGGCGCTCTTCCTCTCTCAGCTGGTGTTCGTGATTGGGATCAACCA
GACGGAAAACCCGTTTCTGTGCACAGTGGTTGCCATCCTCCTCCACTACATCTACATGAGCACCTTTG
CCTGGACCCTCGTGGAGAGCCTGCATGTCTACCGCATGCTGACCGAGGTGCGCAACATCGACACGGGG
CCCATGCGGTTCTACTACTGTCGTGGGCTGGGGATCCCGGCCATTGTCACAGGACTGGCGGTCGGCCT
GGACCCCCAGGGCTACGGGAACCCCGACTTCTGCTGGCTGTCGCTTCAAGACACCCTGATTTGGAGCT
TTGCGGGGCCCATCGGAGCTGTTATAATCATCAACACAGTCACTTCTGTCCTATCTGCAAAGGTTTCC
TGCCAAAGAAAGCACCATTATTATGGGAAAAAAGGGATCGTCTCCCTGCTGAGGACCGCATTCCTCCT
GCTGCTGCTCATCAGCGCCACCTGGCTGCTGGGGCTGCTGGCTGTGAACCGCGATGCACTGAGCTTTC
ACTACCTCTTCGCCATCTTCAGCGGCTTACAGGGCCCCTTCGTCCTCCTTTTCCACTGCGTGCTCAAC
CAGGAGGTCCGGAAGCACCTGAAGGGCGTGCTCGGCGGGAGGAAGCTGCACCTGGAGGACTCCGCCAC
CACCAGGGCCACCCTGCTGACGCGCTCCCTCAACTGCAACACCACCTTCGGTGACGGGCCTGACATGC
TGCGCACAGACTTGGGCGAGTCCACCGCCTCGCTGGACAGCATCGTCAGGGATGAAGGGATCCAGAAG
CTCGGCGTGTCCTCTGGGCTGGTGAGGGGCAGCCACGGAGAGCCAGACGCGTCCCTCATGCCCAGGAG
CTGCAAGGATCCCCCTGGCCACGATTCCGACTCAGATAGCGAGCTGTCCCTGGATGAGCAGAGCAGCT
CTTACGCCTCCTCACACTCGTCAGACAGCGAGGACGATGGGGTGGGAGCTGAGGAAAAATGGGACCCG
GCCAGGGGCGCCGTCCACAGCACACCCAAAGGGGACGCTGTGGCCAACCACGTTCCGGCCGGCTGGCC
CGACCAGAGCCTGGCTGAGAGTGACAGTGAGGACCCCAGCGGCAAGCCCCGCCTGAAGGTGGAGACCA
AGGTCAGCGTGGAGCTGCACCGCGAGGAGCAGGGCAGTCACCGTGGAGAGTACCCCCCGGACCAGGAG
AGCGGGGGCGCAGCCAGGCTTGCTAGCAGCCAGCCCCCAGAGCAGAGGAGCATCTTGAAAAATAAAGT
CACCTACCCGCCGCCGCTGACGCTGACGGAGCAGACGCTGAAGGGCCGGCTCCGGGAGAAGCTGGCCG
ACTGTGAGCAGAGCCCCACATCCTCGCGCACGTCTTCCCTGGGCTCTGGCGGCCCCGACTGCGCCATC
ACAGTCAAGAGCCCTGGGAGGGAGCCGGGGCGTGACCACCTCAACGGGGTGGCCATGAATGTGCGCAC
TGGGAGCGCCCAGGCCGATGGCTCCGACTCTGAGAAACCGTGA

| | | | |
|---|---|---|---|
| NOV21f, SNP13382484 of | SEQ ID NO: 286 | 3028 aa | MW at 330865.9kD |
| CG51965-01, Protein | SNP Pos: 2864 | | SNP Change: Thr to Thr |
| Sequence | | | |

MAPPPPPVLPVLLLLAAAAALPAMGLRAAAWEPRVPGGTRAFALRPGCTYAVGAACTPRAPRELLDVG
RDGRLAGRRRVSGAGRPLPLQVRLVARSAPTALSRRLRARTHLPGCGARARLCGTGARLCGALCFPVP
GGCAAAQHSALAAPTTLPACRCPPRPRPRCPGRPICLPPGGSVRLRLLCALRRAAGAVRVGLALEAAT
AGTPSASPSPSPPLPPNLPEARAGPARRARRGTSGRGSLKFPMPNYQVALFENEPAGTLILQLHAHYT
IEGEEERVSYYMEGLFDERSRGYFRIDSATGAVSTDSVLDRETKETHVLRVKAVDYSTPPRSATTYIT
VLVKDTNDHSPVFEQSEYRERVRENLEVGYEVLTIRASDRDSPINANLRYRVLGGAWDVFQLNESSGV
VSTRAVLDREEAAEYQLLVEANDQGRNPGPLSATATVYIEVEDENDNYPQFSEQNYVVQVPEDVGLNT
AVLRVQATDRDQGQNAAIHYSILSGNVAGQFYLHSLSGILDVINPLDFEDVQKYSLSIKAQDGGRPPL
INSSGVVSVQVLDVNDNEPIFVSSPFQATVLENVPLGYPVVHIQAVDADSGENARLHYRLVDTASTFL
GGGSAGPKNPAPTPDFPFQIHNSSGWITVCAELDREEVEHYSFGVEAVDHGSPPMSSSTSVSITVLDV
NDNDPVFTQPTYELRLNEDAAVGSSVLTLQARDRDANSVITYQLTGGNTRNRFALSSQRGGGLITLAL
PLDYKQEQQYVLAVTASDGTRSHTAHVLINVTDANTNRPVFQSSHYTVSVSEDRPVGTSIATLSANDE
DTGENARITYVIQDPVPQFRIDPDSGTMYTMMELDYENQVAYTLTIMAQDNGIPQKSDTTTLEILILD
ANDNAPQFLWDFYQGSIFEDAPPSTSILQVSATDRDSGPNGRLLYTFQGGDDGDGDFYIEPTSGVIRT
QRRLDRENVAVYNLWAIAVDRGSPTPLSASVEIQVTILDINDNAPMFEKDELELFVEENNPVGSVVAK
IRANDPDEGPNAQIMYQIVEGDMRHFFQLDLLNGDLRAMVELDFEVRREYVLVVQATSAPLVSRATVH
ILLVDQNDNPPVLPDFQILFNNYVTNKSNSFPTGVIGCIPAHDPDVSDSLNYTFVQGNELRLLLLDPA
TGELQLSRDLDNNRPLEALMEVSVSADGIHSVTAFCTLRVTIITDDMLTNSITVRLENMSQEKFLSPL
LALFVEGVAAVLSTTKDDVFVFNVQNDTDVSSNILNVTFSALLPGGVRGQFFPSEDLQEQIYLNRTLL
TTISTQRVLPFDDNICLREPCENYMKCVSVLRFDSSAPFLSSTTVLFRPIHPINGLRCRCPPGFTGDY
CETEIDLCYSDPCGANGRCRSREGGYTCECFEDFTGEHCEVDARSGRCANGVCKNGGTCVNLLIGGFH
CVCPPGEYERPYCEVTTRSFPPQSFVTFRGLRQRFHFTISLTFATQERNGLLLYNGRFNEKHDFIALE
IVDEQVQLTFSAGAGETTTTVAPKVPSGVSDGRWHSVQVQYYNKVRWAPPLPPGPQPNIGHLGLPHGP
SGEKMAVVTVDDCDTTMAVRFGKDIGNYSCAAQGTQTGSKKSLDLTGPLLLGGVPNLPEDFPVHNRQF
VGCMRNLSVDGKNVDMAGFIANNGTREGCAARRNFCDGRRCQNGGTCVNRWNMYLCECPLRFGGKNCE
QANPHPQLFSGESVVSWSDLNIIISVPWYLGLMFRTRKEDSVLMEATSGGPTSFRLQILNNYLQFEVS
HGPSDVESVMLSGLRVTDGEWHHLLIELKNVKEDSEMKHLVTMTLDYGMDQNKADIGGMLPGLTVRSV
VVGGASEDKVSVRRGFRGCMQGVRMGGTPTNVATLNMNNALKVRVKDGCDVDDPCTSSPCPPNSRCHD
AWEDYSCVCDKGYLGINCVDACHLNPCENMGACVRSPGSPQGYVCECGPSHYGPYCENKLDLPCPRGW
WGNPVCGPCHCAVSKGFDPDCNKTNGQCQCKENYYKLLAQDTCLPCDCFPHGSHSRTCDMATGQCACK
PGVIGRQCNRCDNPFAEVTTLGCEVIYNGCPKAFEAGIWWPQTKFGQPAAVPCPKGSVGNAVRHCSGE
KGWLPPELFNCTTISFVDLRAMNEKLSRNETQVDGARALQLVRALRSATQHTGTLFGNDVRTAYQLLG
HVLQHESWQQGFDLAATQDADFHEDVIHSGSALLAPATRAAWEQIQRSEGGTAQLLRRLEGYFSNVAR
NVRRTYLRPFVIVTANMVLAVDIFDKFNFTGARVPRFDTIHEEFPRELESSVSFPADFFRPPEEKEGP
LLRPAGRRTTPQTTRPGPGTEREAPISRRRRHPDDAGQFAVALVIIYRTLGOLLPERYDPDRRSLRLP
HRPIINTPMVSTLVYSEGAPLPRPLERPVLVEFALLEVEERTKPVCVFWNHSLAVGGTGGWSARGCEL
LSRNRTHVACQCSHTASFAVLMDISRRENGEVLPLKIVTYAAVSLSLAALLVAFVLLSLVRMLRSNLH
SIHKHLAVALFLSQLVFVIGINQTENPFLCTVVAILLHYIYMSTFAWTLVESLHVYRMLTEVRNIDTG
PMRFYYVVGWGIPAIVTGLAVGLDPQGYGNPDFCWLSLQDTLIWSFAGPIGAVIIINTVTSVLSAKVS
CQRKHHYYGKKGIVSLLRTAFLLLLLISATWLLGLLAVNRDALSFHYLFAIFSGLQGPFVLLFHCVLN
QEVRKHLKGVLGGRKLHLEDSATTRATLLTRSLNCNTTFGDGPDMLRTDLGESTASLDSIVRDEGIQK
LGVSSGLVRGSHGEPDASLMPRSSKDPPGHDSDSDSELSLDEOSSSYASSHSSDSEDDGVGAEEKWDP
ARGAVHSTPKGDAVANHVPAGWPDQSLAESDSEDPSGKPRLKVETKVSVELHREEQGSHRGEYPPDQE
SGGAARLASSQPPEQRSILKNKVTYPPPLTLTEQTLKGRLREKLADCEQSPTSSRTSSLGSGGPDCAI
TVKSPGREPGRDHLNGVANNVRTGSAQADGSDSEKP

| | | |
|---|---|---|
| NOV21g, SNP13382485 of | SEQ ID NO: 287 | 9087 bp |
| CG51965-01, DNA Sequence | ORF Start: ATG at 1 | ORF Stop: TGA at 9085 |
| | SNP Pos: 8752 | SNP Change: G to C |

ATGGCGCCGCCGCCGCCGCCCGTGCTGCCCGTGCTGCTGCTCCTGGCCGCCGCCGCCGCCCTGCCGGC
GATGGGGCTGCGAGCGGCCGCCTGGGAGCCGCGCGTACCCGGCGGGACCCGCGCCTTCGCCCTCCGGC
CCGGCTGTACCTACGCGGTGGGCGCCGCTTGCACGCCCCGGGCGCCGCGGGAGCTGCTGGACGTGGGC
CGCGATGGGCGGCTGGCAGGACGTCGGCGCGTCTCGGGCGCGGGGCGCCCGCTGCCGCTGCAAGTCCG
CTTGGTGGCCCGCAGTGCCCCGACGGCGCTGAGCCGCCGCCTGCGGGCGCGCACGCACCTTCCCGGCT
GCGGAGCCCGTGCCCGGCTCTGCGGAACCGGTGCCCGGCTCTGCGGGGCGCTCTGCTTCCCCGTCCCC
GGCGGCTGCGCGGCCGCGCAGCATTCGGCGCTCGCAGCTCCGACCACCTTACCCGCCTGCCGCTGCCC
GCCGCGCCCCAGGCCCCGCTGTCCCGGCCGTCCCATCTGCCTGCCGCCGGGCGGCTCGGTCCGCCTGC
GTCTGCTGTGCGCCCTGCGGCGCGCGGCTGGCGCCGTCCGGGTGGGACTGGCGCTGGAGGCCGCCACC
GCGGGGACGCCCTCCGCGTCGCCATCCCCATCGCCGCCCCTGCCGCCGAACTTGCCCGAAGCCCGGGC
GGGGCCGGCGCGACGGGCCCGGCGGGGCACGAGCGGCAGAGGGAGCCTGAAGTTTCCGATGCCCAACT
ACCAGGTGGCGTTGTTTGAGAACGAACCGGCGGGCACCCTCATCCTCCAGCTGCACGCGCACTACACC
ATCGAGGGCGAGGAGGAGCGCGTGAGCTATTACATGGAGGGGCTGTTCGACGAGCGCTCCCGGGGCTA
CTTCCGAATCGACTCTGCCACGGGCGCCGTGAGCACGGACAGCGTACTGGACCGCGAGACCAAGGAGA
CGCACGTCCTCAGGGTGAAAGCCGTGGACTACAGTACGCCGCCGCGCTCGGCCACCACCTACATCACT
GTCTTGGTCAAAGACACCAACGACCACAGCCCGGTCTTCGAGCAGTCGGAGTACCGCGAGCGCGTGCG
GGAGAACCTGGAGGTGGGCTACGAGGTGCTGACCATCCGCGCCAGCGACCGCGACTCGCCCATCAACG
CCAACTTGCGTTACCGCGTGTTGGGGGGCGCGTGGGACGTCTTCCAGCTCAACGAGAGCTCTGGCGTG
GTGAGCACACGGGCGGTGCTGGACCGGGAGGAGGCGGCCGAGTACCAGCTCCTGGTGGAGGCCAACGA
CCAGGGGCGCAATCCGGGCCCGCTCAGTGCCACGGCCACCGTGTACATCGAGGTGGAGGACGAGAACG
ACAACTACCCCCAGTTCAGCGAGCAGAACTACGTGGTCCAGGTGCCCGAGGACGTGGGGCTCAACACG
GCTGTGCTGCGAGTGCAGGCCACGGACCGGGACCAGGGCCAGAACGCGGCCATTCACTACAGCATCCT
CAGCGGGAACGTGGCCGGCCAGTTCTACCTGCACTCGCTGAGCGGGATCCTGGATGTGATCAACCCCT
TGGATTTCGAGGATGTCCAGAAATACTCGCTGAGCATTAAGGCCCAGGATGGGGGCCGGCCCCCGCTC
ATCAATTCTTCAGGGGTGGTGTCTGTGCAGGTGCTGGATGTCAACGACAACGAGCCTATCTTTGTGAG
CAGCCCCTTCCAGGCCACGGTGCTGGAGAATGTGCCCCTGGGCTACCCCGTGGTGCACATTCAGGCGG
TGGACGCGGACTCTGGAGAGAACGCCCGGCTGCACTATCGCCTGGTGGACACGGCCTCCACCTTTCTG
GGGGGCGGCAGCGCTGGGCCTAAGAATCCTGCCCCCACCCCTGACTTCCCCTTCCAGATCCACAACAG
CTCCGGTTGGATCACAGTGTGTGCCGAGCTGGACCGCGAGGAGGTGGAGCACTACAGCTTCGGGGTGG
AGGCGGTGGACCACGGCTCGCCCCCCATGAGCTCCTCCACCAGCGTGTCCATCACGGTGCTGGACGTG
AATGACAACGACCCGGTGTTCACGCAGCCCACCTACGAGCTTCGTCTGAATGAGGATGCGGCCGTGGG
GAGCAGCGTGCTGACCCTGCAGGCCCGCGACCGTGACGCCAACAGTGTGATTACCTACCAGCTCACAG
GCGGCAACACCCGGAACCGCTTTGCACTCAGCAGCCAGAGAGGGGGCGGCCTCATCACCCTGGCGCTA
CCTCTGGACTACAAGCAGGAGCAGCAGTACGTGCTGGCGGTGACAGCATCCGACGGCACACGGTCGCA
CACTGCGCATGTCCTAATCAACGTCACTGATGCCAACACCCACAGGCCTGTCTTTCAGAGCTCCCATT
ACACAGTGAGTGTCAGTGAGGACAGGCCTGTGGGCACCTCCATTGCTACCCTCAGTGCCAACGATGAG
GACACAGGAGAGAATGCCCGCATCACCTACGTGATTCAGGACCCCGTGCCGCAGTTCCGCATTGACCC
CGACAGTGGCACCATGTACACCATGATGGAGCTGGACTATGAGAACCAGGTCGCCTACACGCTGACCA
TCATGGCCCAGGACAACGGCATCCCGCAGAAATCAGACACCACCACCCTAGAGATCCTCATCCTCGAT
GCCAATGACAATGCACCCCAGTTCCTGTGGGATTTCTACCAGGGTTCCATCTTTGAGGATGCTCCACC
CTCGACCAGCATCCTCCAGGTCTCTGCCACGGACCGGGACTCAGGTCCCAATGGGCGTCTGCTGTACA
CCTTCCAGGGTGGGGACGACGGCGATGGGGACTTCTACATCGAGCCCACGTCCGGTGTGATTCGCACC
CAGCGCCGGCTGGACCGGGAGAATGTGGCCGTGTACAACCTTTGGGCTCTGGCTGTGGATCGGGGCAG
TCCCACTCCCCTTAGCGCCTCGGTAGAAATCCAGGTGACCATCTTGGACATTAATGACAATGCCCCCA
TGTTTGAGAAGGACGAACTGGAGCTGTTTGTTGAGGAGAACAACCCAGTGGGGTCGGTGGTGGCAAAG
ATTCGTGCTAACGACCCTGATGAAGGCCCTAATGCCCAGATCATGTATCAGATTGTGGAAGGGGACAT
GCGGCATTTCTTCCAGCTGGACCTGCTCAACGGGGACCTGCGTGCCATGGTGGAGCTGGACTTTGAGG
TCCGGCGGGAGTATGTGCTGGTGGTGCAGGCCACGTCGGCTCCGCTGGTGAGCCGAGCCACGGTGCAC
ATCCTTCTCGTGGACCAGAATGACAACCCGCCTGTGCTGCCCGACTTCCAGATCCTCTTCAACAACTA
TGTCACCAACAAGTCCAACAGTTTCCCCACCGGCGTGATCGGCTGCATCCCGGCCCATGACCCCGACG
TGTCAGACAGCCTCAACTACACCTTCGTGCAGGGCAACGAGCTGCGCCTGTTGCTGCTGGACCCCGCC
ACGGGCGAACTGCAGCTCAGCCGCGACCTGGACAACAACCGGCCGCTGGAGGCGCTCATGGAGGTGTC
TGTGTCTGCAGATGGCATCCACAGCGTCACGGCCTTCTGCACCCTGCGTGTCACCATCATCACGGACG
ACATGCTGACCAACAGCATCACTGTCCGCCTGGAGAACATGTCCCAGGAGAAGTTCCTGTCCCCGCTG
CTGGCCCTCTTCGTGGAGGGGGTGGCCGCCGTGCTGTCCACCACCAAGGACGACGTCTTCGTCTTCAA
CGTCCAGAACGACACCGACGTCAGCTCCAACATCCTGAACGTGACCTTCTCGGCGCTGCTGCCTGGCG
GCGTCCGCGGCCAGTTCTTCCCGTCGGAGGACCTGCAGGAGCAGATCTACCTGAATCGGACGCTGCTG
ACCACCATCTCCACGCAGCGCGTGCTGCCCTTCGACGACAACATCTGCCTGCGCGAGCCCTGCGAGAA
CTACATGAAGTGCGTGTCCGTTCTGCGATTCGACAGCTCCGCGCCCTTCCTCAGCTCCACCACCGTGC
TCTTCCGGCCCATCCACCCCATCAACGGCCTGCGCTGCCGCTGCCCGCCCGGCTTCACCGGCGACTAC
TGCGAGACGGAGATCGACCTCTGCTACTCCGACCCGTGCGGCGCCAACGGCCGCTGCCGCAGCCGCGA
GGGCGGCTACACCTGCGAGTGCTTCGAGGACTTCACTGGAGAGCACTGTGAGGTGGATGCCCGCTCAG
GCCGCTGTGCCAACGGGGTGTGCAAGAACGGGGGCACCTGCGTGAACCTGCTCATCGGCGGCTTCCAC
TGCGTGTGTCCTCCTGGCGAGTATGAGAGGCCCTACTGTGAGGTGACCACCAGGAGCTTCCCGCCCCA
GTCCTTCGTCACCTTCCGGGGCCTGAGACAGCGCTTCCACTTCACCATCTCCCTCACGTTTGCCACTC
AGGAAAGGAACGGCTTGCTTCTCTACAACGGCCGCTTCAATGAGAAGCACGACTTCATCGCCCTGGAG
ATCGTGGACGAGCAGGTGCAGCTCACCTTCTCTGCAGGTGCAGGCGAGACAACAACGACCGTGGCACC
GAAGGTTCCCAGTGGTGTGAGTGACGGGCGGTGGCACTCTGTGCAGGTGCAGTACTACAACAAGGTAA
GATGGGCCCCACCACTTCCCCCTGGCCCCCAGCCCAATATTGGCCACCTGGGCCTGCCCCATGGGCCG
TCCGGGGAAAAGATGGCCGTGGTGACAGTGGATGATTGTGACACAACCATGGCTGTGCGCTTTGGAAA
GGACATCGGGAACTACAGCTGCGCTGCCCAGGGCACTCAGACCGGCTCCAAGAAGTCCCTGGATCTGA
CCGGCCCTCTACTCCTGGGGGGTGTCCCCAACCTGCCAGAAGACTTCCCAGTGCACAACCGGCAGTTC
GTGGGCTGCATGCGGAACCTGTCAGTCGACGGCAAAAATGTGGACATGGCCGGATTCATCGCCAACAA
TGGCACCCGGGAAGGCTGCGCTGCTCGGAGGAACTTCTGCGATGGGAGGCGGTGTCAGAATGGAGGCA
CCTGTGTCAACAGGTGGAATATGTATCTGTGTGAGTGTCCACTCCGATTCGGCGGGAAGAACTGTGAG
CAAGCCATGCCTCACCCCCAGCTCTTCAGCGGTGAGAGCGTCGTGTCCTGGAGTGACCTGAACATCAT
CATCTCTGTGCCCTGGTACCTGGGGCTCATGTTCCGGACCCGGAAGGAGGACAGCGTTCTGATGGAGG
CCACCAGTGGTGGGCCCACCAGCTTTCGCCTCCAGATCCTGAACAACTACCTCCAGTTTGAGGTGTCC
CACGGCCCCTCCGATGTGGAGTCCGTGATGCTGTCCGGGTTGCGGGTGACCGACGGGGAGTGGCACCA
CCTGCTGATCGAGCTGAAGAATGTTAAGGAGGACAGTGAGATGAAGCACCTGGTCACCATGACCTTGG
ACTATGGGATGGACCAGAACAAGGCAGATATCGGGGGCATGCTTCCCGGGCTGACGGTAAGGAGCGTG
GTGGTCGGAGGCGCCTCTGAAGACAAGGTCTCCGTGCGCCGTGGATTCCGAGGCTGCATGCAGGGAGT
GAGGATGGGGGGGACGCCCACCAACGTCGCCACCCTGAACATGAACAACGCACTCAAGGTCAGGGTGA
AGGACGGCTGTGATGTGGACGACCCCTGTACCTCGAGCCCCTGTCCCCCCAATAGCCGCTGCCACGAC
GCCTGGGAGGACTACAGCTGCGTCTGTGACAAAGGGTACCTTGGAATAAACTGTGTGGATGCCTGTCA
CCTGAACCCCTGCGAGAACATGGGGGCCTGCGTGCGCTCCCCCGGCTCCCCGCAGGGCTACGTGTGCG
AGTGTGGGCCCAGTCACTACGGGCCGTACTGTGAGAACAAACTCGACCTTCCGTGCCCCAGAGGCTGG
TGGGGGAACCCCGTCTGTGGACCCTGCCACTGTGCCGTCAGCAAAGGCTTTGATCCCGACTGTAATAA
GACCAACGGCCAGTGCCAATGCAAGGAGAATTACTACAAGCTCCTAGCCCAGGACACCTGTCTGCCCT
GCGACTGCTTCCCCCATGGCTCCCACAGCCGCACTTGCGACATGGCCACCGGGCAGTGTGCCTGCAAG
CCCGGCGTCATCGGCCGCCAGTGCAACCGCTGCGACAACCCGTTTGCCGAGGTCACCACGCTCGGCTG
TGAAGTGATCTACAATGGCTGTCCCAAAGCATTTGAGGCCGGCATCTGGTGGCCACAGACCAAGTTCG
GGCAGCCGGCTGCGGTGCCATGCCCTAAGGGATCCGTTGGAAATGCGGTCCGACACTGCAGCGGGGAG
AAGGGCTGGCTGCCCCCAGAGCTCTTTAACTGTACCACCATCTCCTTCGTGGACCTCAGGGCCATGAA
TGAGAAGCTGAGCCGCAATGAGACGCAGGTGGACGGCGCCAGGGCCCTGCAGCTGGTGAGGGCGCTGC
GCAGTGCTACACAGCACACGGGCACGCTCTTTGGCAATGACGTGCGCACGGCCTACCAGCTGCTGGGC
CACGTCCTTCAGCACGAGAGCTGGCAGCAGGGCTTCGACCTGGCAGCCACGCAGGACGCCGACTTTCA
CGAGGACGTCATCCACTCGGGCAGCGCCCTCCTGGCCCCAGCCACCAGGGCGGCGTGGGAGCAGATCC
AGCGGAGCGAGGGCGGCACGGCACAGCTGCTCCGGCGCCTCGAGGGCTACTTCAGCAACGTGGCACGC
AACGTGCGGCGGACGTACCTGCGGCCCTTCGTCATCGTCACCGCCAACATGGTTCTTGCTGTCGACAT
CTTTGACAAGTTCAACTTTACGGGAGCCAGGGTCCCGCGATTCGACACCATCCATGAAGAGTTCCCCA
GGGAGCTGGAGTCCTCCGTCTCCTTCCCAGCCGACTTCTTCAGACCACCTGAAGAAAAAGAAGGCCCC
CTGCTGAGGCCGGCTGGCCGGAGGACCACCCCGCAGACCACGCGCCCGGGGCCTGGCACCGAGAGGGA
GGCCCCGATCAGCAGGCGGAGGCGACACCCTGATGACGCTGGCCAGTTCGCCGTCGCTCTGGTCATCA
TTTACCGCACCCTGGGGCAGCTCCTGCCCGAGCGCTACGACCCCGACCGTCGCAGCCTCCGGTTGCCT
CACCGGCCCATCATTAATACCCCGATGGTGAGCACGCTGGTGTACAGCGAGGGGGCTCCGCTCCCGAG
ACCCCTGGAGAGGCCCGTCCTGGTGGAGTTCGCCCTGCTGGAGGTGGAGGAGCGAACCAAGCCTGTCT
GCGTGTTCTGGAACCACTCCCTGGCCGTTGGTGGGACGGGAGGGTGGTCTGCCCGGGGCTGCGAGCTC
CTGTCCAGGAACCGGACACATGTCGCCTGCCAGTGCAGCCACACAGCCAGCTTTGCGGTGCTCATGGA
TATCTCCAGGCGTGAGAACGGGGAGGTCCTGCCTCTGAAGATTGTCACCTATGCCGCTGTGTCCTTGT
CACTGGCAGCCCTGCTGGTGGCCTTCGTCCTCCTGAGCCTGGTCCGCATGCTGCGCTCCAACCTGCAC
AGCATTCACAAGCACCTCGCCGTGGCGCTCTTCCTCTCTCAGCTGGTGTTCGTGATTGGGATCAACCA
GACGGAAAACCCGTTTCTGTGCACAGTGGTTGCCATCCTCCTCCACTACATCTACATGAGCACCTTTG
CCTGGACCCTCGTGGAGAGCCTGCATGTCTACCGCATGCTGACCGAGGTGCGCAACATCGACACGGGG
CCCATGCGGTTCTACTACGTCGTGGGCTGGGGCATCCCGGCCATTGTCACAGGACTGGCGGTCGGCCT
GGACCCCCAGGGCTACGGGAACCCCGACTTCTGCTGGCTGTCGCTTCAAGACACCCTGATTTGGAGCT
TTGCGGGGCCCATCGGAGCTGTTATAATCATCAACACAGTCACTTCTGTCCTATCTGCAAAGGTTTCC
TGCCAAAGAAAGCACCATTATTATGGGAAAAAAGGGATCGTCTCCCTGCTGAGGACCGCATTCCTCCT
GCTGCTGCTCATCAGCGCCACCTGGCTGCTGGGGCTGCTGGCTGTGAACCGCGATGCACTGAGCTTTC
ACTACCTCTTCGCCATCTTCAGCGGCTTACAGGGCCCCTTCGTCCTCCTTTTCCACTGCGTGCTCAAC
CAGGAGGTCCGGAAGCACCTGAAGGGCGTGCTCGGCGGGAGGAAGCTGCACCTGGAGGACTCCGCCAC
CACCAGGGCCACCCTGCTGACGCGCTCCCTCAACTGCAACACCACCTTCGGTGACGGGCCTGACATGC
TGCGCACAGACTTGGGCGAGTCCACCGCCTCGCTGGACAGCATCGTCAGGGATGAAGGGATCCAGAAG
CTCGGCGTGTCCTCTGGGCTGGTGAGGGGCAGCCACGGAGAGCCAGACGCGTCCCTCATGCCCAGGAG
CTGCAAGGATCCCCCTGGCCACGATTCCGACTCAGATAGCGAGCTGTCCCTGGATGAGCAGAGCAGCT
CTTACGCCTCCTCACACTCGTCAGACAGCGAGGACGATGGGGTGGGAGCTGAGGAAAAATGGGACCCG
GCCAGGGGCGCCGTCCACAGCACCCCCAAAGGGGACGCTGTGGCCAACCACGTTCCGGCCGGCTGGCC
CGACCAGAGCCTGGCTGAGAGTGACAGTGAGGACCCCAGCGGCAAGCCCCGCCTGAAGGTGGAGACCA
AGGTCAGCGTGGAGCTGCACCGCGAGGAGCAGGGCAGTCACCGTGGACAGTACCCCCCGGACCAGGAG
AGCGGGGGCGCAGCCAGGCTTGCTAGCAGCCAGCCCCCAGAGCAGAGGAGCATCTTGAAAAATAAAGT
CACCTACCCGCCGCCGCTGACGCTGACGGAGCAGACGCTGAAGGGCCGGCTCCGGGAGAAGCTGGCCG
ACTGTGAGCAGAGCCCCACATCCTCGCGCACGTCTTCCCTGGGCTCTGGCGGCCCCGACTGCGCCATC
ACAGTCAAGAGCCCTGGGAGGGAGCCGGGGCGTGACCACCTCAACGGGGTGGCCATGAATGTGCGCAC
TGGGAGCGCCCAGGCCGATGGCTCCGACTCTGAGAAACCGTGA

| | | | |
|---|---|---|---|
| NOV21g, SNP13382485 of | SEQ ID NO: 288 | 3028 aa | MW at 330864.9kD |
| CG51965-01, Protein | SNP Pos: 2918 | | SNP Change: Glu to Gln |
| Sequence | | | |

MAPPPPPVLPVLLLLAAAAALPAMGLRAAAWEPRVPGGTRAFALRPGCTYAVGAACTPRAPRELLDVG
RDGRLAGRRRVSGAGRPLPLQVRLVARSAPTALSRRLRARTHLPGCGARARLCGTGARLCGALCFPVP
GGCAAAQHSALAAPTTLPACRCPPRPRPRCPGRPICLPPGGSVRLRLLCALRRAAGAVRVGLALEAAT
AGTPSASPSPSPPLPPNLPEARAGPARRARRGTSGRGSLKFPMPNYQVALFENEPAGTLILQLHAHYT
IEGEEERVSYYMEGLFDERSRGYFRIDSATGAVSTDSVLDRETKETHVLRVKAVDYSTPPRSATTYIT
VLVKDTNDHSPVFEQSEYRERVRENLEVGYEVLTIRASDRDSPINANLRYRVLGGAWDVFQLNESSGV
VSTRAVLDREEAAEYQLLVEANDQGRNPGPLSATATVYIEVEDENDNYPQFSEQNYVVQVPEDVGLNT
AVLRVQATDRDQGQNAAIHYSILSGNVAGQFYLHSLSGILDVINPLDFEDVQKYSLSIKAQDGGRPPL
INSSGVVSVQVLDVNDNEPIFVSSPFQATVLENVPLGYPVVHIQAVDADSGENARLHYRLVDTASTFL
GGGSAGPKWPAPTPDFPFQIHNSSGWITVCAELDREEVEHYSFGVEAVDHGSPPMSSSTSVSITVLDV
NDNDPVFTQPTYELRINEDAAVGSSVLTLQARDRDANSVITYQLTGGNTRNRFALSSORGGGLITLAL
PLDYKQEQQYVLAVTASDGTRSHTAHVLINVTDANTHRPVFQSSHYTVSVSEDRPVGTSIATLSANDE
DTGENARITYVIQDPVPQFRIDPDSGTMYTMMELDYENQVAYTLTIMAQDNGIPQKSDTTTLEILILD
ANDNAPQFLWDFYQGSIFEDAPPSTSILQVSATDRDSGPNGRLLYTFQGGDDGDGDFYIEPTSGVIRT
QRRLDRENVAVYNLWALAVDRGSPTPLSASVEIQVTILDINDNAPMFEKDELELFVEENNPVGSVVAK
IRANDPDEGPNAQIMYQIVEGDMRHFFQLDLLNGDLRAMVELDFEVRREYVLVVQATSAPLVSRATVH
ILLVDQNDNPPVLPDFQILFNNYVTNKSNSFPTGVIGCIPAHDPDVSDSLNYTFVQGNELRLLLLDPA
TGELQLSRDLDNNRPLEALMEVSVSADGIHSVTAFCTLRVTIITDDMLTNSITVRLENNSQEKFLSPL
LALFVEGVAAVLSTTKDDVFVFNVQNDTDVSSNILNVTFSALLPGGVRGQFFPSEDLQEQIYLNRTLL
TTISTQRVLPFDDNICLREPCENYMKCVSVLRFDSSAPFLSSTTVLFRPIHPINGLRCRCPPGFTGDY
CETEIDLCYSDPCGANGRCRSREGGYTCECFEDFTGEHCEVDARSGRCANGVCKNGGTCVNLLIGGFH
CVCPPGEYERPYCEVTTRSFPPQSFVTFRGLRQRFHFTISLTFATQERNGLLLYNGRFNEKHDFIALE
IVDEQVQLTFSAGAGETTTTVAPKVPSGVSDGRWHSVQVQYYNKVRWAPPLPPGPQPNIGHLGLPHGP
SGEKMAVVTVDDCDTTMAVRFGKDIGNYSCAAQGTQTGSKKSLDLTGPLLLGGVPNLPEDFPVHNRQF
VGCMRNLSVDGKNVDMAGFIANNGTREGCAARRNFCDGRRCQNGGTCVNRWNMYLCECPLRFGGKNCE
QAMPHPQLFSGESVVSWSDLNIIISVPWYLGLMFRTRKEDSVLMEATSGGPTSFRLQILNNYLQFEVS
HGPSDVESVNLSGLRVTDGEWHHLLIELKNVKEDSEMKHLVTMTLDYGMDQNKADIGGMLPGLTVRSV
VVGGASEDKVSVRRGFRGCMQGVRMGGTPTNVATLNNNNALKVRVKDGCDVDDPCTSSPCPPNSRCHD
AWEDYSCVCDKGYLGINCVDACHLNPCENNGACVRSPGSPQGYVCECGPSHYGPYCENKLDLPCPRGW
WGNPVCGPCHCAVSKGFDPDCNKTNGQCQCKENYYKLLAQDTCLPCDCFPHGSHSRTCDMATGOCACK
PGVIGRQCNRCDNPFAEVTTLGCEVIYNGCPKAFEAGIWWPQTKFGQPAAVPCPKGSVGNAVRHCSGE
KGWLPPELFNCTTISFVDLRAMNEKLSRNETQVDGARALQLVRALRSATQHTGTLFGNDVRTAYQLLG
HVLQHESWQQGFDLAATQDADFHEDVIHSGSALLAPATRAAWEQIQRSEGGTAQLLRRLEGYFSNVAR
NVRRTYLRPFVIVTANMVLAVDIFDKFNFTGARVPRFDTIHEEFPRELESSVSFPADFFRPPEEKEGP
LLRPAGRRTTPQTTRPGPGTEREAPISRRRRHPDDAGQFAVALVIIYRTLGQLLPERYDPDRRSLRLP
HRPIINTPMVSTLVYSEGAPLPRPLERPVLVEFALLEVEERTKPVCVFWNHSLAVGGTGGWSARGCEL
LSRNRTHVACQCSHTASFAVLMDISRRENGEVLPLKIVTYAAVSLSLAALLVAFVLLSLVRMLRSNLH
SIHKHLAVALFLSQLVFVIGINQTENPFLCTVVAILLHYIYMSTFAWTLVESLHVYRMLTEVRNIDTG
PMRFYYVVGWGIPAIVTGLAVGLDPQGYGNPDFCWLSLQDTLIWSFAGPIGAVIIINTVTSVLSAKVS
CQRKHHYYGKKGIVSLLRTAFLLLLLISATWLLGLLAVNRDALSFHYLFAIFSGLQGPFVLLFHCVLM
QEVRKHLKGVLGGRKLHLEDSATTRATLLTRSLNCNTTFGDGPDMLRTDLGESTASLDSIVRDEGIQK
LGVSSGLVRGSHGEPDASLMPRSCKDPPGHDSDSDSELSLDEQSSSYASSHSSDSEDDGVGAEEKWDP
ARGAVHSTPKGDAVANHVPAGWPDQSLAESDSEDPSGKPRLKVETKVSVELHREEQGSHRGQYPPDQE
SGGAARLASSQPPEQRSILKNKVTYPPPLTLTEQTLKGRLREKLADCEQSPTSSRTSSLGSGGPDCAI
TVKSPGREPGRDHLNGVANNVRTGSAQADGSDSEKP

A ClustalW comparison of the above protein sequences yields the following sequence alignment shown in Table 21B.

TABLE 21B
Comparison of the NOV21 protein sequences.

NOV21a MAPPPPPVLPVLLLLAAAAALPANGLRAAAWEPRVPGGTRAFALRPGCTYAVGAACTPRA
NOV21b ------------------------------------------------------------
NOV21c ------------------------------------------------------------
NOV21d ------------------------------------------------------------
NOV21a PRELLDVGRDGRLAGRRRVSGAGRPLPLQVRLVARSAPTALSRRLRARTHLPGCGARARL
NOV21b ------------------------------------------------------------
NOV21c ------------------------------------------------------------
NOV21d ------------------------------------------------------------
NOV21a CGTGARLCGALCFPVPGGCAAAQHSALAAPTTLPACRCPPRPRPRCPGRPICLPPGGSVR
NOV21b ------------------------------------------------------------
NOV21c ------------------------------------------------------------
NOV21d ------------------------------------------------------------
NOV21a LRLLCALRRAAGAVRVGLALEAATAGTPSASPSPSPPLPPNLPEARAGPARRARRGTSGR
NOV21b ------------------------------------------------------------
NOV21c ------------------------------------------------------------
NOV21d ------------------------------------------------------------
NOV21a GSLKFPMPNYQVALFENEPAGTLILQLHAHYTIEGEEERVSYYMEGLFDERSRGYFRIDS
NOV21b ------------------------------------------------------------
NOV21c ------------------------------------------------------------
NOV21d -------KLYQVALFENEPAGTLILQLHAHHTIEGEEERVSYYMEGLFDERSRGYFRIDS
NOV21a ATGAVSTDSVLDRETKETHVLRVKAVDYSTPPRSATTYITVLVKDTNDHSPVFEQSEYRE
NOV21b ------------------------------------LKLEVIYNGCPKAFEAGIWWPQTK
NOV21c ------------------------------------------------------------
NOV21d AAGAVSTDSVLDRETKETHVLRVKAVDYSTPPRSATTYITVLVKDTNDHSPVFEQSEYRE
NOV21a RVRENLEVGYEVLTIRASDRDSPINANLRYRVLGGAWDVFQLNESSGVVSTRAVLDREEA
NOV21b FGQPAAVPCPKGSVGNAVRHCSGEKGWLPPELFNCTTISFVDLRAMNEKLSRNETQVDGA
NOV21c --RK-LVLPLKIVTYAAVS-LS-LAALLVAFVLLSLVRMLRSNLHS-IHKHLAVALF--L
NOV21d RVRENLEVGYEVLTIRASDRDSPINANLRYRVLGGAWDVFQLNESSGVVSTRAVLDREEA
NOV21a AEYQLLVEANDQGRNPGPLSATATVYIEVEDENDNYPQFSEQNYVVQVPEDVGLNTAVLR
NOV21b RALQLVRALRSATQHTGTLFGNDVRTAYQLLGHVLQHESWQQGFDLAATQDADFHEDVIH
NOV21c SQLVFVIGIN-QTENP-FLCTVVAILLHYIYMS----TFAWT--LVES-------LHVYR
NOV21d AEYQLLVEANDQGRNPGPLSATATVYIEVEDENDNYPQFSEQNYVVQVPEDVGLNTAVLR
NOV21a VQATDRDQGQNAAIHYSILSGNVAGQFYLHSLSGILDVINPLDFEDVQKYSLSIKAQDGG
NOV21b SGSALLAPATRAAWEQIQRSEG-GTAQLLRRLEGYFSNVARNVRRTYLRPFVIVTANMIL
NOV21c MLTEVRN----------IDTG--PMRFYYVVGWGIPAIVT----G----LAVGLDPQGYG
NOV21d VQATDRDQGQNAAIHYSILSGNVAGQFYLHSLSGILDVINPLDFEDVQKYSLSIKAQDGG
NOV21a RPPLINSSGVVSVQVLDVNDNEPIFVSSPFQATVLENVPLGYPVVHIQAVDADSGENARL
NOV21b AVDIFDKFNFTGARVPRFDTIHEEFPRELESSVSFPADFFRPPEEKEGPLLRPAGRRTTP
NOV21c NPDFC----WLSLQDTLI----WSFAG-PIGAVIIINT--------VTSVLSAKVSCQRK
NOV21d RPPLINSSGVVSVQVLDVNDNEPIFVSSPFQATVLENVPLGYPVVHIQAVDADSGENARL
NOV21a HYRLVDTASTFLGGGSAGPKNPAPTPDFPFQIHNSSGWITVCAELDRE-EVEHYSFGVBA
NOV21b QTTRPGPGTEREAPISRRRRHPDDAGQFAVALVIIYRTLGQLLPERYD-PDRRSLRLPHR
NOV21c HH--------YYG--KKGIVSLLRT-AFLLLLLISATWLLGLLAVNRDALSFHYLFAIFS
NOV21d HYRLVDTASTFLGGGSAGPKNPAPTPDFPFQIRNSSGWITVCAELDREEVE-HYSFGVEA
NOV21a VDHGSPPMSSSTSVSITVLDVNDNDPVFTQPTYELRLNEDAAVGSSVLTLQARDRDANSV
NOV21b PIINTPMVSTLVYSEGAPLPRPLERPVLVEFALLEVEERTKPVCVFWNHSLAVGGTGGWS
NOV21c GLQG-P------FV-LLFHCV-LNQEVKLA------------------------------
NOV21d VDHGWPPMSSSTSVSITVLDVNDNDPVFTQPTYELRLNEDAAVGSSVLTLQARDRDANSV
NOV21a ITYQLTGGNTRNRFALSSQRGGGLITLALPLDYKQEQQYVLAVTASDGTRSHTAHVLINV
NOV21b ARGCELLSRNRTHVACQCSHTASFAVLMDISRRENGEKL---------------------
NOV21c ------------------------------------------------------------
NOV21d ITYQLTGGNTRNRFALSSQRGGGLITLALPLDYKQEQQYVLAVTASDGTRSHTAHVLINV
NOV21a TDANTHRPVFQSSHYTVSVSEDRPVGTSIATLSANDEDTGENARITYVIQDPVPQFRIDP
NOV21b ------------------------------------------------------------
NOV21c ------------------------------------------------------------
NOV21d TDANTHRPVFQSSHYTVSVSEDRPVGTSIATLSANDEDTGENARITYVIQDPVPQFRIDP
NOV21a DSGTMYTMMELDYENQVAYTLTIMAQDNGIPQKSDTTTLEILILDANDNAPQFLWDFYQG
NOV21b ------------------------------------------------------------
NOV21c ------------------------------------------------------------
NOV21d DSGTMYTMMELDYENQVAYTLTIMAQDNGIPQKSDTTTLEILILDANDNAPQFLWDFYQG
NOV21a SIFEDAPPSTSILQVSATDRDSGPNGRLLYTFQGGDDGDGDFYIEPTSGVIRTQRRLDRE
NOV21b ------------------------------------------------------------
NOV21c ------------------------------------------------------------
NOV21d SIFEDAPPSTSILQVSATDRDSGPNGRLLYTFQGGDDGDGDFYIEPTSGVIRTQRRLDRE
NOV21a NVAVYNLWALAVDRGSPTPLSASVEIQVTILDINDNAPMFEKDELELFVEENNPVGSVVA
NOV21b ------------------------------------------------------------
NOV21c ------------------------------------------------------------
NOV21d NVAVYNLWALAVDRGSPTPLSASVEIQVTILDINDNAPMFEKDELELFVEENNPVGSVVA
NOV21a KIRANDPDEGPNAQIMYQIVEGDMRHFFQLDLLNGDLRAMVELDFEVRREYVLVVQATSA
NOV21b ------------------------------------------------------------
NOV21c ------------------------------------------------------------
NOV21d KIRANDPDEGPNAQIMYQIVEGDMRHFFQLDLLNGDLRAMVELDFEVRREYVLVVQATSA
NOV21a PLVSRATVHILLVDQNDNPPVLPDFQILFNNYVTNKSNSFPTGVIGCIPAHDPDVSDSLN
NOV21b ------------------------------------------------------------
NOV21c ------------------------------------------------------------
NOV21d PLVSRATVHILLVLE---------------------------------------------
NOV21a YTFVQGNELRLLLLDPATGELQLSRDLDNNRPLEALMEVSVSADGIHSVTAFCTLRVTII
NOV21b ------------------------------------------------------------
NOV21c ------------------------------------------------------------
NOV21d ------------------------------------------------------------
NOV21a TDDMLTNSITVRLENMSQEKFLSPLLALFVEGVAAVLSTTKDDVFVFNVQNDTDVSSNIL
NOV21b ------------------------------------------------------------
NOV21c ------------------------------------------------------------
NOV21d ------------------------------------------------------------
NOV21a NVTFSALLPGGVRGQFFPSEDLQEQIYLNRTLLTTISTQRVLPFDDNICLREPCENYMKC
NOV21b ------------------------------------------------------------
NOV21c ------------------------------------------------------------
NOV21d ------------------------------------------------------------
NOV21a VSVLRFDSSAPFLSSTTVLFRPIHPINGLRCRCPPGFTGDYCETEIDLCYSDPCGANGRC
NOV21b ------------------------------------------------------------
NOV21c ------------------------------------------------------------
NOV21d ------------------------------------------------------------
NOV21a RSREGGYTCECFEDFTGEHCEVDARSGRCANGVCKNGGTCVNLLIGGFHCVCPPGEYERP
NOV21b ------------------------------------------------------------
NOV21c ------------------------------------------------------------
NOV21d ------------------------------------------------------------
NOV21a YCEVTTRSFPPQSFVTFRGLRQRFHFTISLTFATQERNGLLLYNGRFNEKHDFIALEIVD
NOV21b ------------------------------------------------------------
NOV21c ------------------------------------------------------------
NOV21d ------------------------------------------------------------
NOV21a EQVQLTFSAGAGETTTTVAPKVPSGVSDGRWHSVQVQYYNKVRWAPPLPPGPQPNIGHLG
NOV21b ------------------------------------------------------------
NOV21c ------------------------------------------------------------
NOV21d ------------------------------------------------------------
NOV21a LPHGPSGEKMAVVTVDDCDTTMAVRFGKDIGNYSCAAQGTQTGSKKSLDLTGPLLLGGVP
NOV21b ------------------------------------------------------------
NOV21c ------------------------------------------------------------
NOV21d ------------------------------------------------------------
NOV21a NLPEDFPVHNRQFVGCMRNLSVDGKNVDMAGFIANNGTREGCAARRNFCDGRRCQNGGTC
NOV21b ------------------------------------------------------------
NOV21c ------------------------------------------------------------
NOV21d ------------------------------------------------------------
NOV21a VNRWNMYLCECPLRFGGKNCEQAMPHPQLFSGESVVSWSDLNIIISVPWYLGLMFRTRKE
NOV21b ------------------------------------------------------------
NOV21c ------------------------------------------------------------
NOV21d ------------------------------------------------------------
NOV21a DSVLMEATSGGPTSFRLQILNNYLQFEVSHGPSDVESVMLSGLRVTDGEWHHLLIELKNV
NOV21b ------------------------------------------------------------
NOV21c ------------------------------------------------------------
NOV21d ------------------------------------------------------------
NOV21a KEDSEMKHLVTMTLDYGMDQNXADIGGMLPGLTVRSVVVGGASEDKVSVRRGFRGCMQGV
NOV21b ------------------------------------------------------------
NOV21c ------------------------------------------------------------
NOV21d ------------------------------------------------------------
NOV21a RNGGTPTNVATLNMNNALKVRVKDGCDVDDPCTSSPCPPNSRCHDAWEDYSCVCDKGYLG
NOV21b ------------------------------------------------------------
NOV21c ------------------------------------------------------------
NOV21d ------------------------------------------------------------
NOV21a INCVDACHLNPCENNGACVRSPGSPQGYVCECGPSHYGPYCENKLDLPCPRGWWGNPVCG
NOV21b ------------------------------------------------------------
NOV21c ------------------------------------------------------------
NOV21d ------------------------------------------------------------
NOV21a PCHCAVSKGFDPDCNKTNGQCQCKENYYKLLAQDTCLPCDCFPHGSHSRTCDMATGQCAC
NOV21b ------------------------------------------------------------
NOV21c ------------------------------------------------------------
NOV21d ------------------------------------------------------------
NOV21a KPGVIGRQCNRCDNPFAEVTTLGCEVIYNGCPKAFEAGIWWPQTKFGQPAAVPCPKGSVG
NOV21b ------------------------------------------------------------
NOV21c ------------------------------------------------------------
NOV21d ------------------------------------------------------------
NOV21a NAVRHCSGEKGWLPPELFNCTTISFVDLRAMNEKLSRNETQVDGARALQLVRALRSATQH
NOV21b ------------------------------------------------------------
NOV21c ------------------------------------------------------------
NOV21d ------------------------------------------------------------
NOV21a TGTLFGNDVRTAYQLLGHVLQHESWQQGFDLAATQDADFHEDVIHSGSALLAPATRAAWE
NOV21b ------------------------------------------------------------
NOV21c ------------------------------------------------------------
NOV21d ------------------------------------------------------------
NOV21a QIQRSEGGTAQLLRRLEGYFSNVARNVRRTYLRPFVIVTANNVLAVDIFDKFNFTGARVP
NOV21b ------------------------------------------------------------
NOV21c ------------------------------------------------------------
NOV21d ------------------------------------------------------------
NOV21a RFDTIHEEFPRELESSVSFPADFFRPPEEKEGPLLRPAGRRTTPQTTRPGPGTEREAPIS
NOV21b ------------------------------------------------------------
NOV21c ------------------------------------------------------------
NOV21d ------------------------------------------------------------
NOV21a RRRRHPDDAGQFAVALVIIYRTLGQLLPERYDPDRRSLRLPHRPIINTPMVSTLVYSEGA
NOV21b ------------------------------------------------------------
NOV21c ------------------------------------------------------------
NOV21d ------------------------------------------------------------
NOV21a PLPRPLERPVLVEFALLEVEERTKPVCVFWNHSLAVGGTGGWSARGCELLSRNRTHVACQ
NOV21b ------------------------------------------------------------
NOV21c ------------------------------------------------------------
NOV21d ------------------------------------------------------------
NOV21a CSHTASFAVLMDISRRENGEVLPLKIVTYAAVSLSLAALLVAFVLLSLVRMLRSNLHSIH
NOV21b ------------------------------------------------------------
NOV21c ------------------------------------------------------------
NOV21d ------------------------------------------------------------
NOV21a KHLAVALFLSQLVFVIGINQTENPFLCTVVAILLHYIYMSTFAWTLVESLHVYRMLTEVR
NOV21b ------------------------------------------------------------
NOV21c ------------------------------------------------------------
NOV21d ------------------------------------------------------------
NOV21a NIDTGPMRFYYVVGWGIPAIVTGLAVGLDPQGYGNPDFCWLSLQDTLIWSFAGPIGAVII
NOV21b ------------------------------------------------------------
NOV21c ------------------------------------------------------------
NOV21d ------------------------------------------------------------
NOV21a INTVTSVLSAKVSCQRKHHYYGKKGIVSLLRTAFLLLLLISATWLLGLLAVNRDALSFHY
NOV21b ------------------------------------------------------------
NOV21c ------------------------------------------------------------
NOV21d ------------------------------------------------------------
NOV21a LFAIFSGLQGPFVLLFHCVLNQEVRKHLKGVLGGRKLHLEDSATTRATLLTRSLNCNTTF
NOV21b ------------------------------------------------------------
NOV21c ------------------------------------------------------------
NOV21d ------------------------------------------------------------
NOV21a GDGPDMLRTDLGESTASLDSIVRDEGIQKLGVSSGLVRGSHGEPDASLMPRSCKDPPGHD
NOV21b ------------------------------------------------------------
NOV21c ------------------------------------------------------------
NOV21d ------------------------------------------------------------
NOV21a SDSDSELSLDEQSSSYASSHSSDSEDDGVGAEEKWDPARGAVHSTPKGDAVANHVPAGWP
NOV21b ------------------------------------------------------------
NOV21c ------------------------------------------------------------
NOV21d ------------------------------------------------------------
NOV21a DQSLAESDSEDPSGKPRLKVETKVSVELHREEQGSHRGEYPPDQESGGAARLASSQPPEQ
NOV21b ------------------------------------------------------------
NOV21c ------------------------------------------------------------
NOV21d ------------------------------------------------------------
NOV21a RSILKNKVTYPPPLTLTEQTLKGRLREKLADCEQSPTSSRTSSLGSGGPDCAITVKSPGR
NOV21b ------------------------------------------------------------
NOV21c ------------------------------------------------------------
NOV21d ------------------------------------------------------------
NOV21a EPGRDHLNGVAMNVRTGSAQADGSDSEKP
NOV21b -----------------------------
NOV21c -----------------------------
NOV21d -----------------------------
NOV21a (SEQ ID NO: 276)
NOV21b (SEQ ID NO: 278)
NOV21c (SEQ ID NO: 280)
NOV21d (SEQ ID NO: 282)

Further analysis of the NOV21a protein yielded the following properties shown in Table 21C.

TABLE 21C
Protein Sequence Properties NOV21a

SignalP analysis: Cleavage site between residues 21 and 22
PSORT II analysis:

| PSG: | a new signal peptide prediction method |
|---|---|
| | N-region: length 0; pos.chg 0; neg.chg 0 |
| | H-region: length 26; peak value 9.94 |
| | PSG score: 5.54 |
| GvH: | von Heijne's method for signal seq. recognition |
| | GvH score (threshold: −2.1): 3.49 |
| | possible cleavage site: between 20 and 21 |

>>> Seems to have a cleavable signal peptide (1 to 20)

| ALOM: | Klein et al's method for TM region allocation | |
|---|---|---|
| | Init position for calculation: 21 | |
| | Tentative number of TMS(s) for the threshold 0.5: 10 | |
| | INTEGRAL Likelihood = −4.99 | Transmembrane 1220 −1236 |
| | INTEGRAL Likelihood = −0.53 | Transmembrane 2251 −2267 |
| | INTEGRAL Likelihood = −0.06 | Transmembrane 2351 −2367 |
| | INTEGRAL Likelihood = −9.92 | Transmembrane 2489 −2505 |
| | INTEGRAL Likelihood = −5.95 | Tranamembrane 2521 −2537 |
| | INTEGRAL Likelihood = −1.54 | Transmembrane 2544 −2560 |
| | INTEGRAL Likelihood = −2.81 | Transmembrane 2591 −2607 |
| | INTEGRAL Likelihood = −1.59 | Transmembrane 2631 −2647 |
| | INTEGRAL Likelihood = −8.92 | Transmembrane 2674 −2690 |
| | INTEGRAL Likelihood = −1.17 | Transmembrane 2703 −2719 |

PERIPHERAL Likelihood = 2.81 (at 1076)
ALOM score: −9.92 (number of TMSs: 10)

MTOP: Prediction of membrane topology (Hartmann et al.)

Center position for calculation: 10
Charge difference: 1.0 C( 2.0) - N( 1.0)
C > N: C-terminal side will be inside

>>> Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75) | 1.33 |
| Hyd Moment(95): | 2.60 | G content: | 1 |
| D/E content: | 1 | S/T content: | 0 |
| Score: −5.63 | | | |

Gavel: prediction of cleavage sites for mitochondrial preseq

R-2 motif at 69 PRA|PR

NUCDISC: discrimination of nuclear localization signals

pat4: RRRR (5) at 2340
pat4: RRRH (3) at 2341
pat7: PARRARR (4) at 229
pat7: PISRRRR (5) at 2337
bipartite: none
content of basic residues: 9.1%
NLS Score: 0.80

KDEL: ER retention motif in the C-terminus: none
ER Membrane Retention Signals:

KKXX-like motif in the C-terminus: DSEK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | Prediction: cytoplasmic |
| | Reliability: 70.6 |
| COIL: | Lupas's algorithm to detect coiled-coil regions |
| | total: 0 residues |
| | -------------------------- |

Final Results (k = 9/23):

66.7%: endoplasmic reticulum
11.1%: vacuolar
11.1%: mitochondrial
11.1%: cytoplasmic

>> prediction for CG51965-01 is end (k = 9)

A search of the NOV21a protein against the Geneseq database, a proprietary database that contains sequences published in patents and patent publication, yielded several homologous proteins shown in Table 21D.

TABLE 21D
Geneseq Results for NOV21a

| | | NOV21a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE08586 | Human NOV7 protein - Homo | 1 . . . 3028 | 3028/3028 (100%) | 0.0 |
| | sapiens, 3028 aa. | 1 . . . 3028 | 3028/3028 (100%) | |
| | [WO200161009-A2, 23-AUG- | | | |
| | 2001] | | | |
| ABP81979 | Human GPCR | 1 . . . 3028 | 3011/3029 (99%) | 0.0 |
| | CELSR1/Flamingo protein | 1 . . . 3014 | 3012/3029 (99%) | |
| | SEQ ID NO: 444 - Homo | | | |
| | sapiens, 3014 aa. | | | |
| | [WO200261087-A2, 08-AUG- | | | |
| | 2002] | | | |
| AAU02196 | Seven-pass transmembrane | 1 . . . 3028 | 3011/3029 (99%) | 0.0 |
| | receptor-like protein, MEM1 - | 1 . . . 3014 | 3012/3029 (99%) | |
| | Homo sapiens, 3014 aa. | | | |
| | [WO200144473-A2, 21-JUN- | | | |
| | 2001] | | | |
| AAU68533 | Human novel cytokine encoded | 1 . . . 3028 | 3011/3029 (99%) | 0.0 |
| | by cDNA 790C1P2C_4 #1 - | 1 . . . 3014 | 3012/3029 (99%) | |
| | Homo sapiens, 3014 aa. | | | |
| | [WO200175093-A1, 11-OCT- | | | |
| | 2001] | | | |
| AAW27161 | Mouse receptor ME2 - Mus | 256 . . . 2902 | 2138/2647 (80%) | 0.0 |
| | musculus, 2707 aa. | 35 . . . 2663 | 2345/2647 (87%) | |
| | [WO9707209-A2, 27-FEB- | | | |
| | 1997] | | | |

In a BLAST search of public sequence databases, the NOV21a protein was found to have homology to the proteins shown in the BLASTP data in Table 21E.

TABLE 21E
Public BLASTP Results for NOV21a

| | | NOV21a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NYQ6 | Cadherin EGF LAG seven-pass | 1 . . . 3028 | 3011/3029 (99%) | 0.0 |
| | G-type receptor 1 precursor | 1 . . . 3014 | 3012/3029 (99%) | |
| | (Flamingo homolog 2) (hFmi2) - | | | |
| | Homo sapiens (Human), 3014 aa. | | | |
| O35161 | Cadherin EGF LAG seven-pass | 1 . . . 3028 | 2453/3057 (80%) | 0.0 |
| | G-type receptor 1 precursor - Mus | 1 . . . 3034 | 2680/3057 (87%) | |
| | musculus (Mouse), 3034 aa. | | | |
| E1260972 | SEQUENCE 3 FROM PATENT | 256 . . . 2902 | 2138/2647 (80%) | 0.0 |
| | WO9707209 - unidentified, 2707 | 35 . . . 2663 | 2345/2647 (87%) | |
| | aa. | | | |
| Q9HCU4 | Cadherin EGF LAG seven-pass | 223 . . . 3026 | 1658/2839 (58%) | 0.0 |
| | G-type receptor 2 precursor | 163 . . . 2915 | 2067/2839 (72%) | |
| | (Epidermal growth factor-like 2) | | | |
| | (Multiple epidermal growth | | | |
| | factor-like domains 3) (Flamingo | | | |
| | 1) - Homo sapiens (Human), 2923 | | | |
| | aa. | | | |
| Q9R0M0 | Cadherin EGF LAG seven-pass | 2 . . . 3026 | 1665/3072 (54%) | 0.0 |
| | G-type receptor 2 precursor | 8 . . . 2912 | 2095/3072 (67%) | |
| | (Flamingo 1) (mFmi1) - Mus | | | |
| | musculus (Mouse), 2920 aa. | | | |

PFam analysis indicates that the NOV21a protein contains the domains shown in the Table 21F.

TABLE 21F
Domain Analysis of NOV21a

| | | Identities/ | |
|---|---|---|---|
| | NOV21a | Similarities | Expect |
| Pfam Domain | Match Region | for the Matched Region | Value |
| cadherin | 250 . . . 344 | 35/110 (32%) | 1e-14 |
| | | 70/110 (64%) | |
| cadherin | 358 . . . 450 | 44/107 (41%) | 1.8e-21 |
| | | 72/107 (67%) | |
| cadherin | 464 . . . 556 | 38/107 (36%) | 2e-24 |
| | | 74/107 (69%) | |
| cadherin | 570 . . . 678 | 39/124 (31%) | 3.8e-23 |
| | | 87/124 (70%) | |
| cadherin | 692 . . . 780 | 35/107 (33%) | 7.4e-19 |
| | | 71/107 (66%) | |
| cadherin | 794 . . . 883 | 42/107 (39%) | 1.6e-28 |
| | | 73/107 (68%) | |
| cadherin | 897 . . . 990 | 41/108 (38%) | 2.2e-27 |
| | | 76/108 (70%) | |
| cadherin | 1004 . . . 1092 | 38/107 (36%) | 3.8e-22 |
| | | 67/107 (63%) | |
| EGF | 1308 . . . 1361 | 16/61 (26%) | 0.71 |
| | | 38/61 (62%) | |
| EGF | 1368 . . . 1399 | 15/47 (32%) | 1.8e-05 |
| | | 26/47 (55%) | |
| EGF | 1408 . . . 1441 | 19/47 (40%) | 5.3e-07 |
| | | 29/47 (62%) | |
| laminin_G | 1471 . . . 1647 | 43/206 (21%) | 5.5e-17 |
| | | 138/206 (67%) | |
| EGF | 1668 . . . 1699 | 17/47 (36%) | 4.7e-07 |
| | | 24/47 (51%) | |
| laminin_G | 1734 . . . 1867 | 26/163 (16%) | 0.0034 |
| | | 90/163 (55%) | |
| EGF | 1891 . . . 1922 | 11/47 (23%) | 7.7e-05 |
| | | 24/47 (51%) | |
| EGF | 1926 . . . 1960 | 18/47 (38%) | 0.0057 |
| | | 24/47 (51%) | |
| laminin_EGF | 1981 . . . 2015 | 13/60 (22%) | 0.87 |
| | | 24/60 (40%) | |
| laminin_EGF | 2018 . . . 2063 | 26/59 (44%) | 8.8e-09 |
| | | 31/59 (53%) | |
| HRM | 2067 . . . 2124 | 21/77 (27%) | 5.1e-17 |
| | | 47/77 (61%) | |
| GPS | 2422 . . . 2475 | 26/56 (46%) | 1.6e-21 |
| | | 46/56 (82%) | |
| 7tm_2 | 2480 . . . 2723 | 88/274 (32%) | 6.4e-90 |
| | | 210/274 (77%) | |

### Example 22

The NOV22 clone was analyzed, and the nucleotide and encoded polypeptide sequences are shown in Table 22A.

TABLE 22A
NOV22 Sequence Analysis

| | | |
|---|---|---|
| NOV22a, CG51983-05 | SEQ ID NO: 289 | 2268 bp |
| DNA Sequence | ORF Start: ATG at 1 | ORF Stop: TAG at 2266 |

ATGCTTCCCGGGTGTATATTCTTGATGATTTTACTCATTCCTCAGGTTAAAGAAAAGTTCATCCTTGG
AGTAGAGGGTCAACAACTGGTTCGTCCTAAAAAGCTTCCTCTGATACAGAAGCGAGATACTGGACACA
CCCATGATGATGACATACTGAAAACGTATGAAGAAGAATTGTTGTATGAAATAAAACTAAATAGAAAA
ACCTTAGTCCTTCATCTTCTAAGATCCAGGGAGTTCCTAGGCTCAAATTACAGTGAAACATTCTACTC
CATGAAAGGAGAAGCGTTCACCAGGCATCCTCAGATCATGGAACACTGTTACTATAAAGGAAACATCC
TAAATGAAAAGAATTCTGTTGCCAGCATCAGTACTTGTGACGGGTTGAGGGGATTCTTCAGAATAAAC
GACCAAAGATACCTCATTGAACCAGTGAAATACTCAGATGAGGGAGAACATTTGGTGTTCAAATATAA
CCTGAGGGTGCCGTATGGTGCCAATTATTCCTGTACAGAGCTTAATTTTACCAGAAAAACTGTTCCAG
GGGATAATGAATCTGAAGAAGACTCCAAAATAAAAGGCATCCATGATGAAAAGTATGTTGAATTGTTC
ATTGTTGCTGATGATACTGTGTATCGCAGAAATGGTCATCCTCACAATAAACTAAGGAACCGAATTTG
GGGAATGGTCAATTTTGTCAACATGATTTATAAAACCTTAAACATCCATGTGACGTTGGTTGGCATTG
AAATATGGACACATGAAGATAAATAGAACTATATTCAAATATAGAAACTACCTTATTGCGTTTTTTCA
TTTTGGCAAGAAAAGATCCTTAAAACACGGAAGGATTTTGATCATGTTGTATTACTCAGTGGGAAGTG
GCTCTACTCACATGTGCAAGGAATTTCTTATCCAGGGGGTATGTGCCTGCCCTATTATTCCACCAGTA
TCATTAAGGATCTTTTACCTGACACAAACATAATTGCAAACAGAATGGCACATCAACTGGGGCATAAC
CTTGGGATGCAGCATGACGAGTTCCCATGCACCTGTCCTTCAGGAAAATGCGTGATGGACAGTGATGG
AAGCATTCCTGCACTGAAATTCAGTAAATGCAGCCAAAACCAATACCACCAGTACTTGAAGGATTATA
AGCCAACATGCATGCTCAACATTCCATTTCCTTACAATTTTCATGATTTCCAATTTTGTGGAAACAAG
AAGTTGGATGAGGGTGAAGAGTGTGACTGTGGCCCTGCTCAGGAGTGTACTAATCCTTGCTGTGATGC
ACACACATGTGTACTGAAGCCAGGATTTACTTGTGCAGAAGGAGAATGCTGTGAATCTTGTCAGATAA
AAAAAGCAGGGTCCATATGCAGACCGGCGAAAGATGAATGTGATTTTCCTGAGATGTGCACTGGCCAC
TCGCCTGCCTGTCCTAAGGACCAGTTCAGGGTCAATGGATTTCCTTGCAAGAACTCAGAAGGCTACTG
TTTCATGGGGAAATGTCCAACTCGTGAGGATCAGTGCTCTGAACTATTTGATGATGAGGCAATAGAGA
GTCATGATATCTGCTACAAGATGAATACAAAAGGAAATAAATTTGGATACTGCAAAAACAAGGAAAAC
AGATTTCTTCCCTGTGAGGAGAAGGATGTCAGATGTGGAAAGATCTACTGCACTGGAGGGGAGCTTTC
CTCTCTCCTTGGAGAAGACAAGACTTATCACCTTAAGGATCCCCAGAAGAATGCTACTGTCAAATGCA
AAACTATTTTTTTATACCATGATTCTACAGACATTGGCCTGGTGGCGTCAGGAACAAAATGTGGAGAG
GGAATGGTATGCAACAATGGTGAATGTCTAAACATGGAAAAGGTCTATATCTCAACCAATTGCCCCTC
TCAGTGCAATGAAAATCCTGTAGATGGCCACGGACTCCAGTGCCACTGTGAGGAAGGACAGGCACCTG
TAGCCTGTGAAGAAACCTTACATGTTACCAGTATCACCATCTTGGTTGTTGTGCTTGTCCTGGTTATT
GTCGGTATCGGAGTTCTTATACTATTAGTTCGTTACCGAAAATGTATCAAGTTGAAGCAAGTTCAGAG
CCCACCTACAGAAACCCTGGGAGTGGAGAACAAAGGATACTTTGGTGATGAGCAGCAGATAAGGACTG
AGCCAATCCTGCCAGAAATTCATTTCCTAAATAAACCTGCAAGTAAAGATTCAAGAGGAATCGCAGAT
CCCAATCAAAGTGCCAAGTGGTAG

| | | | |
|---|---|---|---|
| NOV22a, CG51983-05 | SEQ ID NO: 290 | 755 aa | MW at 85868.5kD |
| Protein Sequence | | | |

MLPGCIFLMILLIPQVKEKFILGVEGQQLVRPKKLPLIQKRDTGHTHDDDILKTYEEELLYEIKLNRK
TLVLHLLRSREFLGSNYSETFYSMKGEAFTRHPQIMEHCYYKGNILNEKNSVASISTCDGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRMRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCABGECCESCQIKKAGSICRPAKDECDFPEMCTGH
SPACPKDQFRVNGFPCKNSEGYCFMGKCPTREDQCSELFDDEAIESHDICYKMNTKGNKFGYCKNKEN
RFLPCEEKDVRCGKIYCTGGELSSLLGEDKTYHLKDPQKNATVKCKTIFLYHDSTDIGLVASGTKCGE
GMVCNNGECLNMEKVYISTNCPSQCNENPVDGHGLQCHCEEGQAPVACEETLHVTSITILVVVLVLVI
VGIGVLILLVRYRKCIKLKQVQSPPTETLGVENKGYFGDEQQIRTEPILPEIHFLNKPASKDSRGIAD
PNQSAKW

| | | |
|---|---|---|
| NOV22b, CG51983-01 | SEQ ID NO: 291 | 2431 bp |
| DNA Sequence | ORF Start: ATG at 51 | ORF Stop: TAG at 2385 |

GAACTCCTTTTCTCAAGCACTTCTGCTCTCCTCTACCAGAATCACTCAGAATGCTTCCCGGGTGTATA
TTCTTGATGATTTTACTCATTCCTCAGGTTAAAGAAAAGTTCATCCTTGGAGTAGAGGGTCAACAACT
GGTTCGTCCTAAAAAGCTTCCTCTGATACAGAAGCGAGATACTGGACACACCCATGATGATGACATAA
AAACGTATGAAGAAGAATTGTTGTATGAAATAAAACTAAATAGAAAAACCTTAGTCCTTCATCTTCTA
AGATCCAGGAGGGAGTTCCTAGGCTCAAATTACAGTGAAACATTCTACTCCATGAAAGGAGAAGCGTT
CACCAGGCATCCTCAGATCATGGATCATTGTTTTTACCAAGGATCCATAGTACACGAATATGATTCAG
CTGCCAGTATCAGTACGTGTAATGGTCTAAGGGGATTCTTCAGAATAAACGACCAAAGATACCTCATT
GAACCAGTGAAATACTCAGATGAGGGAGAACATTTGGTGTTCAAATATAACCTGAGGGTGCCGTATGG
TGCCAATTATTCCTGTACAGAGCTTAATTTTACCAGAAAAACTGTTCCAGGGGATAATGAATCTGAAG
AAGACTCCAAAATAAAACAGGGCATCCATGATGAAAAGTATGTTGAATTGTTCATTGTTGCTGATGAT
ACTGTGTATCGCAGAAATGGTCATCCTCACAATAAACTAAGGAACCGAATTTGGGGAATGGTCAATTT
TGTCAACATGATTTATAAAACCTTAAACATCCATGTGACGTTGGTTGGCATTGAAATATGGACACATG
AAGATAAAATAGAACTATATTCAAATATAGAAACTACCTTATTGCGTTTTTCATTTTGGCAAGAAAAG
ATCCTTAAAACACGGAAGGATTTTGATCATGTTGTATTACTCAGTGGGAAGTGGCTCTACTCACATGT
GCAAGGAATTTCTTATCCAGGGGGTATGTGCCTGCCCTATTATTCCACCAGTATCATTAAGGATCTTT
TACCTGACACAAACATAATTGCAAACAGAATGGCACATCAACTGGGGCATAACCTTGGGATGCAGCAT
GACGAGTTCCCATGCACCTGTCCTTCAGGAAAATGCGTGATGGACAGTGATGGAAGCATTCCTGCACT
GAAATTCAGTAAATGCAGCCAAAACCAATACCACCAGTACTTGAAGGATTATAAGCCAACATGCATGC
TCAACATTCCATTTCCTTACAATTTTCATGATTTCCAATTTTGTGGAAACAAGAAGTTGGATGAGGGT
GAAGAGTGTGACTGTGGCCCTGCTCAGGAGTGTACTAATCCTTGCTGTGATGCACACACATGTGTACT
GAAGCCAGGATTTACTTGTGCAGAAGGAGAATGCTGTGAATCTTGTCAGATAAAAAAAGCAGGGTCCA
TATGCAGACCGGCGAAAGATGAATGTGATTTTCCTGAGATGTGCACTGGCCACTCGCCTGCCTGTCCT
AAGGACCAGTTCAGGGTCAATGGATTTCCTTGCAAGAACTCAGAAGGCTACTGTTTCATGGGGAAATG
TCCAACTCGTGAGGATCAGTGCTCTGAACTATTTGATGATGAGGCAATAGAGAGTCATGATATCTGCT
ACAAGATGAATACAAAAGGAAATAAATTTGGATACTGCAAAAACAAGGAAAACAGATTTCTTCCCTGT
GAGGAGAAGGATGTCAGATGTGGAAAGATCTACTGCACTGGAGGGGAGCTTTCCTCTCTCCTTGGAGA
AGACAAGACTTATCACCTTAAGGATCCCCAGAAGAATGCTACTGTCAAATGCAAAACTATTTTTTTAT
ACCATGATTCTACAGACATTGGCCTGGTGGCGTCAGGAACAAAATGTGGAGAGGGAATGGTATGCAAC
AATGGTGAATGTCTAAACATGGAAAAGGTCTATATCTCAACCAATTGCCCCTCTCAGTGCAATGAAAA
TCCTGTAGATGGCCACGGACTCCAGTGCCACTGTGAGGAAGGACAGGCACCTGTAGCCTGTGAAGAAA
CCTTACATGTTACCAGTATCACCATCTTGGTTGTTGTGCTTGTCCTGGTTATTGTCGGTATCGGAGTT
CTTATACTATTAGTTCGTTACCGAAAATGTATCAAGTTGAAGCAAGTTCAGAGCCCACCTACAGAAAC
CCTGGGAGTGGAGAACAAAGGATACTTTGGTGATGAGCAGCAGATAAGGACTGAGCCAATCCTGCCAG
AAATTCATTTCCTAAATCAGAGAACTCCAGAATCCTTGGAAAGCCTGCCCACTAGTTTTTCAAGTCCC
CACTACATCACACTGAAACCTGCAAGTAAAGATTCAAGAGGAATCGCAGATCCCAATCAAAGTGCCAA
GTGGTAGGTTACCCTGACAGATAGTACCTCCCTTTTTTATTTTTCAAATGC

| | | | |
|---|---|---|---|
| NOV22b, GG51983-01 | SEQ ID NO: 292 | 778 aa | MW at 88471.2kD |
| Protein Sequence | | | |

MLPGCIFLMILLIPQVKEKFILGVEGQQLVRPKKLPLIQKRDTGHTHDDDIKTYEEELLYEIKLNRKT
LVLHLLRSRREFLGSNYSETFYSMKGEAFTRHPQIMDHCFYQGSIVHEYDSAASISTCNGLRGFFRIN
DQRYLIEPVKYSDEGEHLVFKYNLRVPYGANYSCTELNFTRKTVPGDNESEEDSKIKQGIHDEKYVEL
FIVADDTVYRRNGHPHNKLRNRIWGMVNFVNMIYKTLNIHVTLVGIEIWTHEDKIELYSNIETTLLRF
SFWQEKILKTRKDFDHVVLLSGKWLYSHVQGISYPGGMCLPYYSTSIIKDLLPDTNIIANRMAHQLGH
NLGMQHDEFPCTCPSGKCVMDSDGSIPALKFSKCSQNQYHQYLKDYKPTCMLNIPFPYNFHDFQFCGN
KKLDEGEECDCGPAQECTNPCCDAHTCVLKPGFTCAEGECCESCQIKKAGSICRPAKDECDFPEMCTG
HSPACPKDQFRVNGFPCKNSEGYCFMGKCPTREDQCSELFDDEAIESHDICYKMNTKGNKFGYCKNKE
NRFLPCEEKDVRCGKIYCTGGELSSLLGEDKTYHLKDPQKNATVKCKTIFLYHDSTDIGLVASGTKCG
EGMVCNNGECLNMEKVYISTNCPSQCNENPVDGHGLQCHCEEGQAPVACEETLHVTSITILVVVLVLV
IVGIGVLILLVRYRKCIKLKQVQSPPTETLGVENKGYFGDEQQIRTEPILPEIHFLNQRTPESLESLP
TSFSSPHYITLKPASKDSRGIADPNQSAKW

| | | |
|---|---|---|
| NOV22c, GG51983-02 | SEQ ID NO: 293 | 2583 bp |
| DNA Sequence | ORF Start: ATG at 85 | ORF Stop: TGA at 2347 |

GATCCCTGCAGTGGAAGTGAGGAGGAAGAAAGGTGAACTCCTTTTCTCAAGCACTTCTGCTCTCCTCT
ACCAGAATCACTCAGAATGCTTCCCGGGTGTATATTCTTGATGATTTTACTCATTCCTCAGGTTAAAG
AAAAGTTCATCCTTGGAGTAGAGGGTCAACAACTGGTTCGTCCTAAAAAGCTTCCTCTGATACAGAAG
CGAGATACTGGACACACCCATGATGATGACATACTGAAAACGTATGAAGAAGAATTGTTGTATGAAAT
AAAACTAAATAGAAAAACCTTAGTCCTTCATCTTCTAAGATCCAGGGAGTTCCTAGGCTCAAATTACA
GTGAAACATTCTACTCCATGAAAGGAGGAGCGTTCACCAGGCATCCTCAGATCATGGATCATTGTTTT
TACCAAGGATCCATAGTACACGAATATGATTCAGCTGCCAGTATCAGTACGTGTAATGGTCTAAGGGG
ATTCTTCAGAATAAACGACCAAAGATACCTCATTGAACCAGTGAAATACTCAGATGAGGGAGAACATT
TGGTGTTCAAATATAACCTGAGGGTGCCGTATGGTGCCAATTATTCCTGTACAGAGCTTAATTTTACC
AGAAAAACTGTTCCAGGGGATAATGAATCTGAAGAAGACTCCAAAATAAAAGGCATCCATGATGAAAA
GTATGTTGAATTGTTCATTGTTGCTGATGATACTGTGTATCGCAGAAATGGTCATCCTCACAATAAAC
TAAGGAACCGAATTTGGGGAATGGTCAATTTTGTCAACATGATTTATAAAACCTTAAACATCCATGTG
ACGTTGGTTGGCATTGAAATATGGACACATGAAGATAAAATAGAACTATATTCAAATATAGAAACTAC
CTTATTGCGTTTTTCATTTTGGCAAGAAAAGATCCTTAAAACACGGAAGGATTTTGATCATGTTGTAT
TACTCAGTGGGAAGTGGCTCTACTCACATGTGCAAGGAATTTCTTATCCAGGGGGTATGTGCCTGCCC
TATTATTCCACCAGTATCATTAAGGATCTTTTACCTGACACAAACATAATTGCAAACAGAATGGCACA
TCAACTGGGGCATAACCTTGGGATGCAGCATGACGAGTTCCCATGCACCTGTCCTTCAGGAAAATGCG
TGATGGACAGTGATGGAAGCATTCCTGCACTGAAATTCAGTAAATGCAGCCAAAACCAATACCACCAG
TACTTGAAGGATTATAAGCCAACATGCATGCTCAACATTCCATTTCCTTACAATTTTCATGATTTCCA
ATTTTGTGGAAACAAGAAGTTGGATGAGGGTGAAGAGTGTGACTGTGGCCCTGCTCAGGAGTGTACTA
ATCCTTGCTGTGATGCACACACATGTGTACTGAAGCCAGGATTTACTTGTGCAGAAGGAGAATGCTGT
GAATCTTGTCAGATAAAAAAAGCAGGGTCCATATGCAGACCGGCGAAAGATGAATGTGATTTTCCTGA
GATGTGCACTGGCCACTCGCCTGCCTGTCCTAAGGACCAGTTCAGGGTCAATGGATTTCCTTGCAAGA
ACTCAGAAGGCTACTGTTTCATGGGGAAATGTCCAACTCGTGAGGATCAGTGCTCTGAACTATTTGAT
GATGATGCAATAGAGAGTCATGATATCTGCTACAAGATGAATACAAAAGGAAATAAATTTGGATACTG
CAAAAACAAGGAAAACAGATTTCTTCCCTGTGAGGAGAAAGATGTCAGATGTGGAAAGATCTACTGCA
CTGGAGGGGAGCTTTCCTCTCTCCTTGGAGAAGACAAGACTTATCACCTTAAGGATCCCCAGAAGAAT
GCTACTGTCAAATGCAAAACTATTTTTTTATACCATGATTCTACAGACATTGGCCTGGTGGCGTCAGG
AACAAAATGTGGAGAGGGAATGGTGTGCAACAATGGTGAATGTCTAAACATGGAAAAGGTCTATATCT
CAACCAATTGCCCCTCTCAGTGCAATGAAAATCCTGTGGATGGCCACGGACTCCAGTGCCACTGTGAG
GAAGGACAGGCACCTGTAGCCTGTGAAGAAACCTTACATGTTACCAATATCACCATCTTGGTTGTTGT
GCTTGTCCTGGTTATTGTCGGTATCGGAGTTCTTATACTATTAGTTCGTTACCGAAAATGTATCAAGT
TGAAGCAAGTTCAGAGCCCACCTACAGAAACCCTGGGAGTGGAGAACAAAGGATACTTTGGTGATGAG
CAGCAGATAAGGACTGAGCCAATCCTGCCAGAAATTCATTTCCTAAATAAACCTGCAAGTAAAGATTC
AAGAGGAATCGCAGATCCCAATCAAAGTGCCAAGTGAGCTTGAAGTTGGATATCCAAAATGGCCGTGC
AAGCTTAGGCTGGGGATTCTGGATGCAACGTCTTTACAACCTTACCTAGATATCTGCTACTCACATTT
TTGGTAGTGTTTCAAACGTTCTTTATCCAGACAGACAATGTTTAAGAGAAACAACTTATTTCTGTTAA
TATTTACCGGTAGAATTCACACCCTCTATCATAAACATATGCTGCAGAAAAAAAAAAAAAAAAAAAA

| | | | |
|---|---|---|---|
| NOV22c, CG51983-02 | SEQ ID NO: 294 | 754 aa | MW at 85582.1kD |
| Protein Sequence | | | |

MLPGCIFLMILLIPQVKEKFILGVEGQQLVRPKKLPLIQKRDTGHTHDDDILKTYEEELLYEIKLNRK
TLVLHLLRSREFLGSNYSETFYSMKGGAFTRHPQIMDHCFYQGSIVHEYDSAASISTCNGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCAEGECCESCQIKKAGSICRPAKDECDFPEMCTGH
SPACPKDQFRVNGFPCKNSEGYCFMGKCPTREDQCSELFDDDAIESHDICYKMNTKGNKFGYCKWKEN
RFLPCEEKDVRCGKIYCTGGELSSLLGEDKTYHLKDPQKNATVKCKTIFLYHDSTDIGLVASGTKCGE
GMVCNNGECLNMEKVYISTNCPSQCNENPVDGHGLQCHCEEGQAPVACEETLHVTNITILVVVLVLVI
VGIGVLILLVRYRKCIKLKQVQSPPTETLGVENKGYFGDEQQIRTEPILPEIHFLNKPASKDSRGIAD
PNQSAK

| | | |
|---|---|---|
| NOV22d, GG51983-03 | SEQ ID NO: 295 | 2274 bp |
| DNA Sequence | ORF Start: ATG at 4 | ORF Stop: TAG at 2272 |

AGAATGCTTCCCGGGTGTATATTCTTGATGATTTTACTCATTCCTCAGGTTAAAGAAAAGTTCATCCT
TGGAGTAGAGGGTCAACAACTGGTTCGTCCTAAAAAGCTTCCTCTGATACAGAAGCGAGATACTGGAC
ACACCCATGATGATGACATAAAAAACGTATGAAGAAGAATTGTTGTATGAATAAAACTAAATAGAAAA
ACCTTAGTCCTTCATCTTCTAAGATCCAGGAGGGAGTTCCTAGGCTCAAATTACAGTGAAACATTCTA
CTCCATGAAAGGAGAAGCGTTCACCAGGCATCCTCAGATCATGGATCATTGTTTTTACCAAGGATCCA
TAGTACACGAATATGATTCAGCTGCCAGTATCAGTACGTGTAATGGTCTAAGGGGATTCTTCAGAATA
AACGACCAAAGATACCTCATTGAACCAGTGAAATACTCAGATGAGGGAGAACATTTGGTGTTCAAATA
TAACCTGAGGGTGCCGTATGGTGCCAATTATTCCTGTACAGAGCTTAATTTTACCAGAAAAACTGTTC
CAGGGGATAATGAATCTGAAGAAGACTCCAAAATAAAACAGGGCATCCATGATGAAAAGTATGTTGAA
TTGTTCATTGTTGCTGATGATACTGTGTATCGCAGAAATGGTCATCCTCACAATAAACTAAGGAACCG
AATTTGGGGAATGGTCAATTTTGTCAACATGATTTATAAAACCTTAAACATCCATGTGACGTTGGTTG
GCATTGAAATATGGACACATGAAGATAAAATAGAACTATATTCAAATATAGAAACTACCTTATTGCGT
TTTTCATTTTGGCAAGAAAAGATCCTTAAAACACGGAAGGATTTTGATCATGTTGTATTACTCAGTGG
GAAGTGGCTCTACTCACATGTGCAAGGAATTTCTTATCCAGGGGGTATGTGCCTGCCCTATTATTCCA
CCAGTATCATTAAGGATCTTTTACCTGACACAAACATAATTGCAAACAGAATGGCACATCAACTGGGG
CATAACCTTGGGATGCAGCATGACGAGTTCCCATGCACCTGTCCTTCAGGAAAATGCGTGATGGACAG
TGATGGAAGCATTCCTGCACTGAAATTCAGTAAATGCAGCCAAAACCAATACCACCAGTACTTGAAGG
ATTATAAGCCAACATGCATGCTCAACATTCCATTTCCTTACAATTTTCATGATTTCCAATTTTGTGGA
AACAAGAAGTTGGATGAGGGTGAAGAGTGTGACTGTGGCCCTGCTCAGGAGTGTACTAATCCTTGCTG
TGATGCACACACATGTGTACTGAAGCCAGGATTTACTTGTGCAGAAGGAGAATGCTGTGAATCTTGTC
AGATAAAAAAAGCAGGGTCCATATGCAGACCGGCGAAAGATGAATGTGATTTTCCTGAGATGTGCACT
GGCCACTCGCCTGCCTGTCCTAAGGACCAGTTCAGGGTCAATGGATTTCCTTGCAAGAACTCAGAAGG
CTACTGTTTCATGGGGAAATGTCCAACTCGTGAGGATCAGTGCTCTGAACTATTTGATGATGAGGCAA
TAGAGAGTCATGATATCTGCTACAAGATGAATACAAAAGGAAATAAATTTGGATACTGCAAAAACAAG
GAAAACAGATTTCTTCCCTGTGAGGAGAAGGATGTCAGATGTGGAAAGATCTACTGCACTGGAGGGAA
GCTTTCCTCTCTCCTTGGAGAAGACAAGACTTATCACCTTAAGGATCCCCAGAAGAATGCTACTGTCA
AATGCAAAACTATTTTTTTATACCATGATTCTACAGACATTGGCCTGGTGGCGTCAGGAACAAAATGT
GGAGAGGGAATGGTATGCAACAATGGTGAATGTCTAAACATGGAAAAGGTCTATATCTCAACCAATTG
CCCCTCTCAGTGCAATGAAAATCCTGTAGATGGCCACGGACTCCAGTGCCACTGTGAGGAAGGACAGG
CACCTGTAGCCTGTGAAGAAACCTTACATGTTACCAGTATCACCATCTTGGTTGTTGTGCTTGTCCTG
GTTATTGTCGGTATCGGAGTTCTTATACTATTAGTTCGTTACCGAAAATGTATCAAGTTGAAGCAAGT
TCAGAGCCCACCTACAGAAACCCTGGGAGTGGAGAACAAAGGATACTTTGGTGATGAGCAGCAGATAA
GGACTGAGCCAATCCTGCCAGAAATTCATTTCCTAAATAAACCTGCAAGTAAAGATTCAAGAGGAATC
GCAGATCCCAATCAAAGTGCCAAGTGGTAG

| | | | |
|---|---|---|---|
| NOV22d, CG51983-03 | SEQ ID NO: 296 | 756 aa | MW at 85998.5kD |
| Protein Sequence | | | |

MLPGCIFLMILLIPQVKEKFILGVEGQQLVRPKKLPLIQKRDTGHTHDDDIKTYEEELLYEIKLNRKT
LVLHLLRSRREFLGSNYSETFYSMKGEAFTRHPQIMDHCFYQGSIVHEYDSAASISTCNGLRGFFRIN
DQRYLIEPVKYSDEGEHLVFKYNLRVPYGANYSCTELNFTRKTVPGDNESEEDSKIKQGIHDEKYVEL
FIVADDTVYRRNGHPHNKLRNRIWGMVNFVNMIYKTLNIHVTLVGIEIWTHEDKIELYSNIETTLLRF
SFWQEKILKTRKDFDHVVLLSGKWLYSHVQGISYPGGMCLPYYSTSIIKDLLPDTNIIANRMAHQLGH
NLGMQHDEFPCTCPSGKCVMDSDGSIPALKFSKCSQNQYHQYLKDYKPTCMLNIPFPYNFHDFQFCGN
KKLDEGEECDCGPAQECTNPCCDAHTCVLKPGFTCAEGECCESCQIKKAGSICRPAKDECDFPEMCTG
HSPACPKDQFRVNGFPCKNSEGYCFMGKCPTREDQCSELFDDEAIESHDICYKMNTKGNKFGYCKNKE
NRFLPCEEKDVRCGKIYCTGGELSSLLGEDKTYHLKDPQKNATVKCKTIFLYHDSTDIGLVASGTKCG
EGMVCNNGECLNMEKVYISTNCPSQCNENPVDGHGLQCHCEEGQAPVACEETLHVTSITILVVVLVLV
IVGIGVLILLVRYRKCIKLKQVQSPPTETLGVENKGYFGDEQQIRTEPILPEIHFLNKPASKDSRGIA
DPNQSAKW

| | | |
|---|---|---|
| NOV22e, CG51983-04 | SEQ ID NO: 297 | 2283 bp |
| DNA Sequence | ORF Start: ATG at 12 | ORF Stop: TGA at 2274 |

AATCACTCAGAATGCTTCCCGGGTGTATATTCTTGATGATTTTACTCATTCCTCAGGTTAAAGAAAAG
TTCATCCTTGGAGTAGAGGGTCAACAACTGGTTCGTCCTAAAAAGCTTCCTCTGATACAGAAGCGAGA
TACTGGACACACCCATGATGATGACATACTGAAAACGTATGAAGAAGAATTGTTGTATGAAATAAAAC
TAAATAGAAAAACCTTAGTCCTTCATCTTCTAAGATCCAGGGAGTTCCTAGGCTCAAATTACAGTGAA
ACATTCTACTCCATGAAAGGAGGAGCGTTCACCAGGCATCCTCAGATCATGGATCATTGTTTTTACCA
AGGATCCATAGTACACGAATATGATTCAGCTGCCAGTATCAGTACGTGTAATGGTCTAAGGGGATTCT
TCAGAATAAACGACCAAAGATACCTCATTGAACCAGTGAAATACTCAGATGAGGGAGAACATTTGGTG
TTCAAATATAACCTGAGGGTGCCGTATGGTGCCAATTATTCCTGTACAGAGCTTAATTTTACCAGAAA
AACTGTTCCAGGGGATAATGAATCTGAAGAAGACTCCAAAATAAAAGGCATCCATGATGAAAAGTATG
TTGAATTGTTCATTGTTGCTGATGATACTGTGTATCGCAGAAATGGTCATCCTCACAATAAACTAAGG
AACCGAATTTGGGGAATGGTCAATTTTGTCAACATGATTTATAAAACCTTAAACATCCATGTGACGTT
GGTTGGCATTGAAATATGGACACATGAAGATAAAATAGAACTATATTCAAATATAGAAACTACCTTAT
TGCGTTTTTCATTTTGGCAAGAAAAGATCCTTAAAACACGGAAGGATTTTGATCATGTTGTATTACTC
AGTGGGAAGTGGCTCTACTCACATGTGCAAGGAATTTCTTATCCAGGGGGTATGTGCCTGCCCTATTA
TTCCACCAGTATCATTAAGGATCTTTTACCTGACACAAACATAATTGCAAACAGAATGGCACATCAAC
TGGGGCATAACCTTGGGATGCAGCATGACGAGTTCCCATGCACCTGTCCTTCAGGAAAATGCGTGATG
GACAGTGATGGAAGCATTCCTGCACTGAAATTCAGTAAATGCAGCCAAAACCAATACCACCAGTACTT
GAAGGATTATAAGCCAACATGCATGCTCAACATTCCATTTCCTTACAATTTTCATGATTTCCAATTTT
GTGGAAACAAGAAGTTGGATGAGGGTGAAGAGTGTGACTGTGGCCCTGCTCAGGAGTGTACTAATCCT
TGCTGTGATGCACACACATGTGTACTGAAGCCAGGATTTACTTGTGCAGAAGGAGAATGCTGTGAATC
TTGTCAGATAAAAAAAGCAGGGTCCATATGCAGACCGGCGAAAGATGAATGTGATTTTCCTGAGATGT
GCACTGGCCACTCGCCTGCCTGTCCTAAGGACCAGTTCAGGGTCAATGGATTTCCTTGCAAGAACTCA
GAAGGCTACTGTTTCATGGGGAAATGTCCAACTCGTGAGGATCAGTGCTCTGAACTATTTGATGATGA
TGCAATAGAGAGTCATGATATCTGCTACAAGATGAATACAAAAGGAAATAAATTTGGATACTGCAAAA
ACAAGGAAAACAGATTTCTTCCCTGTGAGGAGAAAGATGTCAGATGTGGAAAGATCTACTGCACTGGA
GGGGAGCTTTCCTCTCTCCTTGGAGAAGACAAGACTTATCACCTTAAGGATCCCCAGAAGAATGCTAC
TGTCAAATGCAAAACTATTTTTTTATACCATGATTCTACAGACATTGGCCTGGTGGCGTCAGGAACAA
AATGTGGAGAGGGAATGGTGTGCAACAATGGTGAATGTCTAAACATGGAAAAGGTCTATATCTCAACC
AATTGCCCCTCTCAGTGCAATGAAAATCCTGTGGATGGCCACGGACTCCAGTGCCACTGTGAGGAAGG
ACAGGCACCTGTAGCCTGTGAAGAAACCTTACATGTTACCAATATCACCATCTTGGTTGTTGTGCTTG
TCCTGGTTATTGTCGGTATCGGAGTTCTTATACTATTAGTTCGTTACCGAAAATGTATCAAGTTGAAG
CAAGTTCAGAGCCCACCTACAGAAACCCTGGGAGTGGAGAACAAAGGATACTTTGGTGATGAGCAGCA
GATAAGGACTGAGCCAATCCTGCCGGAAATTCATTTCCTAAATAAACCTGCAAGTAAAGATTCAAGAG
GAATCGCAGATCCCAATCAAAGTGCCAAGTGAGCTTGAA

| | | | |
|---|---|---|---|
| NOV22e, CG51983-04 | SEQ ID NO: 298 | 754 aa | MW at 85582.1kD |
| Protein Sequence | | | |

MLPGCIFLMILLIPQVKEKFILGVEGQQLVRPKKLPLIQKRDTGHTHDDDILKTYEEELLYEIKLNRK
TLVLHLLRSREFLGSNYSETFYSMKGGAFTRHPQIMDHCFYQGSIVHEYDSAASISTCNGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCAEGECCESCQIKKAGSICRPAKDECDFPEMCTGH
SPACPKDQFRVNGFPCKNSEGYCFMGKCPTREDQCSELFDDDAIESHDICYKMNTKGNKFGYCKNKEN
RFLPCEEKDVRCGKIYCTGGELSSLLGEDKTYHLKDPQKNATVKCKTIFLYHDSTDIGLVASGTKCGE
GMVCNNGECLNNEKVYISTNCPSQCNENPVDGHGLQCHCEEGQAPVACEETLHVTNITILVVVLVLVI
VGIGVLILLVRYRKCIKLKQVQSPPTETLGVENKGYFGDEQQIRTEPILPEIHFLNKPASKDSRGIAD
PNQSAK

| | | |
|---|---|---|
| NOV22f, CG51983-06 | SEQ ID NO: 299 | 2464 bp |
| DNA Sequence | ORF Start: ATG at 4 | ORF Stop: TGA at 2248 |

AGAATGCTTCCCGGGTGTATATTCTTGATGATTTTACTCATTCCTCAGGTTAAAGAAAAGTTCATCCT
TGGAGTAGAGGGTCAACAACTGGTTCGTCCTAAAAAGCTTCCTCTGATACAGAAGCGAGATACTGGAC
ACACCCATGATGATGACATACTGAAAACGTATGAAGAAGAATTGTTGTATGAAATAAAACTAAATAGA
AAAACCTTAGTCCTTCATCTTCTAAGATCCAGGGAGTTCCTAGGCTCAAATTACAGTGAAACATTCTA
CTCCATGAAAGGAGAAGCGTTCACCAGGCATCCTCAGATCATGGATCATTGTTTTTACCAAGGATCCA
TAGTACACGAATATGATTCAGCTGCCAGTATCAGTACGTGTAATGGTCTAAGGGGATTCTTCAGAATA
AACGACCAAAGATACCTCATTGAACCAGTGAAATACTCAGATGAGGGAGAACATTTGGTGTTCAAATA
TAACCTGAGGGTGCCGTATGGTGCCAATTATTCCTGTACAGAGCTTAATTTTACCAGAAAAACTGTTC
CAGGGGATAATGAATCTGAAGAAGACTCCAAAATAAAAGGCATCCATGATGAAAAGTATGTTGAATTG
TTCATTGTTGCTGATGATACTGTGTATCGCAGAAATGGTCATCCTCACAATAAACTAAGGAACCGAAT
TTGGGGAATGGTCAATTTTGTCAACATGATTTATAAAACCTTAAACATCCATGTGACGTTGGTTGGCA
TTGAAATATGGACACATGAAGATAAAATAGAACTATATTCAAATATAGAAACTACCTTATTGCGTTTT
TCATTTTGGCAAGAAAAGATCCTTAAAACACGGAAGGATTTTGATCATGTTGTATTACTCAGTGGGAA
GTGGCTCTACTCACATGTGCAAGGAATTTCTTATCCAGGGGGTATGTGCCTGCCCTATTATTCCACCA
GTATCATTAAGGATCTTTTACCTGACACAAACATAATTGCAAACAGAATGGCACATCAACTGGGGCAT
AACCTTGGGATGCAGCATGACGAGTTCCCATGCACCTGTCCTTCAGGAAAATGCGTGATGGACAGTGA
TGGAAGCATTCCTGCACTGAAATTCAGTAAATGCAGCCAAAACCAATACCACCAGTACTTGAAGGATT
ATAAGCCAACATGCATGCTCAACATTCCATTTCCTTACAATTTTCATGATTTCCAATTTTGTGGAAAC
AAGAAGTTGGATGAGGGTGAAGAGTGTGACTGTGGCCCTGCTCAGGAGTGTACTAATCCTTGCTGTGA
TGCACACACATGTGTACTGAAGCCAGGATTTACTTGTGCAGAAGGAGAATGCTGTGAATCTTGTCAGA
TAAAAAAAGCAGGGTCCATATGCAGACCGGCGAAAGATGAATGTGATTTTCCTGAGATGTGCACTGGC
CACTCGCCTGCCTGTCCTAAGGACCAGTTCAGGGTCAATGGATTTCCTTGCAAGAACTCAGAAGGCTA
CTGTTTCATGGGGAAATGTCCAACTCGTGAGGATCAGTGCTCTGAACTATTTGATGATGAGGCAATAG
AGAGTCATGATATCTGCTACAAGATGAATACAAAAGGAAATAAATTTGGATACTGCAAAAACAAGGAA
AACAGATTTCTTCCCTGTGAGGAGAAAGATGTCAGATGTGGAAAGACCTACTGCACTGGAGGGGAGCT
TTCCTCTCTCCTTGGAGAAGACAAGACTTATCACCTTAAGGATCCCCAGAAGAATGCTACTGTCAAAT
GCAAAACTATTTTTTTATACCATGATTCTACAGACATTGGCCTGGTGGCGTCAGGAACAAAATGTGGA
GAGGGAATGGTGGATGGCCACGGACTCCAGTGCCACTGTGAGGAAGGACAGGCACCTGTAGCCTGTGA
AGAAACCTTACATGTTACCAATATCACCATCTTGGTTGTTGTGCTTGTCCTGGTTATTGTCGGTATCG
GAGTTCTTATACTATTAGTTCGTTACCGAAAATGTATCAAGTTGAAGCAAGTTCAGAGCCCACCTACA
GAAACCCTGGGAGTGGAGAACAAAGGATACTTTGGTGATGAGCAGCAGATAAGGACTGAGCCAATCCT
GCCAGAAATTCATTTCCTAAATAGAACTCCAGAATCCTTGGAAAGCCTGCCCACTAGTTTTTCAAGTC
CCCACTACATCACACTGAAACCTGCAAGTAAAGATTCAAGAGGAATCGCAGATCCCAATCAAAGTGCC
AAGTGAGCTTGAAGTTGGATATCCAAAATGGCCGTGCAAGCTTAGGCTGGGGATTCTGGATGCAACGT
CTTTACAACCTTACCTAGATATCTGCTACTCACATTTTTGGTAGTGTTTCAAACGTTCTTTATCCAGA
CAGACAATGTTTAAGAGAAACAACTTATTTCTGTTAATATTTACCGGTAGAATTCACACCCTCTATCA
TAAACATATGCTGCAG

| | | | |
|---|---|---|---|
| NOV22f, CG51983-06 | SEQ ID NO: 300 | 748 aa | MW at 85014.4kD |
| Protein Sequence | | | |

MLPGCIFLMILLIPQVKEKFILGVEGQQLVRPKKLPLIQKRDTGHTHDDDILKTYEEELLYEIKLNRK
TLVLHLLRSREFLGSNYSETFYSMKGEAFTRHPQIMDHCFYQGSIVHEYDSAASISTCNGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCAEGECCESCQIKKAGSICRPAKDECDFPEMCTGH
SPACPKDQFRVNGFPCKNSEGYCFMGKCPTREDQCSELFDDEAIESHDICYKMNTKGNKFGYCKNKEN
RFLPCEEKDVRCGKTYCTGGELSSLLGEDKTYHLKDPQKNATVKCKTIFLYHDSTDIGLVASGTKCGE
GMVDGHGLQCHCEEGQAPVACEETLHVTNITILVVVLVLVIVGIGVLILLVRYRKCIKLKQVQSPPTE
TLGVENKGYFGDEQQIRTEPILPEIHFLNRTPESLESLPTSFSSPHYITLKPASKDSRGIADPNQSAK

| | | |
|---|---|---|
| NOV22g, SNP13376585 | SEQ ID NO: 301 | 2268 bp |
| of GG51983-05, DNA | ORF Start: ATG at 1 | ORF Stop: TAG at 2266 |
| Sequence | SNP Pos: 119 | SNP Change: A to G |

ATGCTTCCCGGGTGTATATTCTTGATGATTTTACTCATTCCTCAGGTTAAAGAAAAGTTCATCCTTGG
AGTAGAGGGTCAACAACTGGTTCGTCCTAAAAAGCTTCCTCTGATACAGAGGCGAGATACTGGACACA
CCCATGATGATGACATACTGAAAACGTATGAAGAAGAATTGTTGTATGAAATAAAACTAAATAGAAAA
ACCTTAGTCCTTCATCTTCTAAGATCCAGGGAGTTCCTAGGCTCAAATTACAGTGAAACATTCTACTC
CATGAAAGGAGAAGCGTTCACCAGGCATCCTCAGATCATGGAACACTGTTACTATAAAGGAAACATCC
TAAATGAAAAGAATTCTGTTGCCAGCATCAGTACTTGTGACGGGTTGAGGGGATTCTTCAGAATAAAC
GACCAAAGATACCTCATTGAACCAGTGAAATACTCAGATGAGGGAGAACATTTGGTGTTCAAATATAA
CCTGAGGGTGCCGTATGGTGCCAATTATTCCTGTACAGAGCTTAATTTTACCAGAAAAACTGTTCCAG
GGGATAATGAATCTGAAGAAGACTCCAAAATAAAAGGCATCCATGATGAAAAGTATGTTGAATTGTTC
ATTGTTGCTGATGATACTGTGTATCGCAGAAATGGTCATCCTCACAATAAACTAAGGAACCGAATTTG
GGGAATGGTCAATTTTGTCAACATGATTTATAAAACCTTAAACATCCATGTGACGTTGGTTGGCATTG
AAATATGGACACATGAAGATAAAATAGAACTATATTCAAATATAGAAACTACCTTATTGCGTTTTTCA
TTTTGGCAAGAAAAGATCCTTAAAACACGGAAGGATTTTGATCATGTTGTATTACTCAGTGGGAAGTG
GCTCTACTCACATGTGCAAGGAATTTCTTATCCAGGGGGTATGTGCCTGCCCTATTATTCCACCAGTA
TCATTAAGGATCTTTTACCTGACACAAACATAATTGCAAACAGAATGGCACATCAACTGGGGCATAAC
CTTGGGATGCAGCATGACGAGTTCCCATGCACCTGTCCTTCAGGAAAATGCGTGATGGACAGTGATGG
AAGCATTCCTGCACTGAAATTCAGTAAATGCAGCCAAAACCAATACCACCAGTACTTGAAGGATTATA
AGCCAACATGCATGCTCAACATTCCATTTCCTTACAATTTTCATGATTTCCAATTTTGTGGAAACAAG
AAGTTGGATGAGGGTGAAGAGTGTGACTGTGGCCCTGCTCAGGAGTGTACTAATCCTTGCTGTGATGC
ACACACATGTGTACTGAAGCCAGGATTTACTTGTGCAGAAGGAGAATGCTGTGAATCTTGTCAGATAA
AAAAAGCAGGGTCCATATGCAGACCGGCGAAAGATGAATGTGATTTTCCTGAGATGTGCACTGGCCAC
TCGCCTGCCTGTCCTAAGGACCAGTTCAGGGTCAATGGATTTCCTTGCAAGAACTCAGAAGGCTACTG
TTTCATGGGGAAATGTCCAACTCGTGAGGATCAGTGCTCTGAACTATTTGATGATGAGGCAATAGAGA
GTCATGATATCTGCTACAAGATGAATACAAAAGGAAATAAATTTGGATACTGCAAAAACAAGGAAAAC
AGATTTCTTCCCTGTGAGGAGAAGGATGTCAGATGTGGAAAGATCTACTGCACTGGAGGGGAGCTTTC
CTCTCTCCTTGGAGAAGACAAGACTTATCACCTTAAGGATCCCCAGAAGAATGCTACTGTCAAATGCA
AAACTATTTTTTTATACCATGATTCTACAGACATTGGCCTGGTGGCGTCAGGAACAAAATGTGGAGAG
GGAATGGTATGCAACAATGGTGAATGTCTAAACATGGAAAAGGTCTATATCTCAACCAATTGCCCCTC
TCAGTGCAATGAAAATCCTGTAGATGGCCACGGACTCCAGTGCCACTGTGAGGAAGGACAGGCACCTG
TAGCCTGTGAAGAAACCTTACATGTTACCAGTATCACCATCTTGGTTGTTGTGCTTGTCCTGGTTATT
GTCGGTATCGGAGTTCTTATACTATTAGTTCGTTACCGAAAATGTATCAAGTTGAAGCAAGTTCAGAG
CCCACCTACAGAAACCCTGGGAGTGGAGAACAAAGGATACTTTGGTGATGAGCAGCAGATAAGGACTG
AGCCAATCCTGCCAGAAATTCATTTCCTAAATAAACCTGCAAGTAAAGATTCAAGAGGAATCGCAGAT
CCCAATCAAAGTGCCAAGTGGTAG

| | | | |
|---|---|---|---|
| NOV22g, SNP13376585 of | SEQ ID NO: 302 | 755 aa | MW at 85896.5k.D |
| CG51983-05, Protein | SNP Pos: 40 | | SNP Change: Lys to Arg |
| Sequence | | | |

MLPGCIFLMILLIPQVKEKFILGVEGQQLVRPKKLPLIQRRDTGHTHDDDILKTYEEELLYEIKLNRK
TLVLHLLRSREFLGSNYSETFYSMKGEAFTRHPQIMEHCYYKGNILNEKNSVASISTCDGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCABGECCESCQIKKAGSICRPAKDECDFPEMCTGH
SPACPKDQFRVNGFPCKNSEGYCFMGKCPTREDQCSELFDDEAIESHDICYKMNTKGNKFGYCKNKEN
RFLPCEEKDVRCGKIYCTGGELSSLLGEDKTYHLKDPQKNATVKCKTIFLYHDSTDIGLVASGTKCGE
GMVCNNGECLNMEKVYISTNCPSQCNENPVDGHGLQCHCEEGQAPVACEETLHVTSITILVVVLVLVI
VGIGVLILLVRYRKCIKLKQVQSPPTETLGVENKGYFGDEQQIRTEPILPEIHFLNKPASKDSRGIAD
PNQSAKW

| | | |
|---|---|---|
| NOV22h, SNP13376586 of | SEQ ID NO: 303 | 2268 bp |
| CG51983-05, DNA Sequence | ORF Start: ATG at 1 | ORF Stop: TAG at 2266 |
| | SNP Pos: 125 | SNP Change: A to G |

ATGCTTCCCGGGTGTATATTCTTGATGATTTTACTCATTCCTCAGGTTAAAGAAAAGTTCATCCTTGG
AGTAGAGGGTCAACAACTGGTTCGTCCTAAAAAGCTTCCTCTGATACAGAAGCGAGGTACTGGACACA
CCCATGATGATGACATACTGAAAACGTATGAAGAAGAATTGTTGTATGAAATAAAACTAAATAGAAAA
ACCTTAGTCCTTCATCTTCTAAGATCCAGGGAGTTCCTAGGCTCAAATTACAGTGAAACATTCTACTC
CATGAAAGGAGAAGCGTTCACCAGGCATCCTCAGATCATGGAACACTGTTACTATAAAGGAAACATCC
TAAATGAAAAGAATTCTGTTGCCAGCATCAGTACTTGTGACGGGTTGAGGGGATTCTTCAGAATAAAC
GACCAAAGATACCTCATTGAACCAGTGAAATACTCAGATGAGGGAGAACATTTGGTGTTCAAATATAA
CCTGAGGGTGCCGTATGGTGCCAATTATTCCTGTACAGAGCTTAATTTTACCAGAAAAACTGTTCCAG
GGGATAATGAATCTGAAGAAGACTCCAAAATAAAAGGCATCCATGATGAAAAGTATGTTGAATTGTTC
ATTGTTGCTGATGATACTGTGTATCGCAGAAATGGTCATCCTCACAATAAACTAAGGAACCGAATTTG
GGGAATGGTCAATTTTGTCAACATGATTTATAAAACCTTAAACATCCATGTGACGTTGGTTGGCATTG
AAATATGGACACATGAAGATAAAATAGAACTATATTCAAATATAGAAACTACCTTATTGCGTTTTTCA
TTTTGGCAAGAAAAGATCCTTAAAACACGGAAGGATTTTGATCATGTTGTATTACTCAGTGGGAAGTG
GCTCTACTCACATGTGCAAGGAATTTCTTATCCAGGGGGTATGTGCCTGCCCTATTATTCCACCAGTA
TCATTAAGGATCTTTTACCTGACACAAACATAATTGCAAACAGAATGGCACATCAACTGGGGCATAAC
CTTGGGATGCAGCATGACGAGTTCCCATGCACCTGTCCTTCAGGAAAATGCGTGATGGACAGTGATGG
AAGCATTCCTGCACTGAAATTCAGTAAATGCAGCCAAAACCAATACCACCAGTACTTGAAGGATTATA
AGCCAACATGCATGCTCAACATTCCATTTCCTTACAATTTTCATGATTTCCAATTTTGTGGAAACAAG
AAGTTGGATGAGGGTGAAGAGTGTGACTGTGGCCCTGCTCAGGAGTGTACTAATCCTTGCTGTGATGC
ACACACATGTGTACTGAAGCCAGGATTTACTTGTGCAGAAGGAGAATGCTGTGAATCTTGTCAGATAA
AAAAAGCAGGGTCCATATGCAGACCGGCGAAAGATGAATGTGATTTTCCTGAGATGTGCACTGGCCAC
TCGCCTGCCTGTCCTAAGGACCAGTTCAGGGTCAATGGATTTCCTTGCAAGAACTCAGAAGGCTACTG
TTTCATGGGGAAATGTCCAACTCGTGAGGATCAGTGCTCTGAACTATTTGATGATGAGGCAATAGAGA
GTCATGATATCTGCTACAAGATGAATACAAAAGGAAATAAATTTGGATACTGCAAAAACAAGGAAAAC
AGATTTCTTCCCTGTGAGGAGAAGGATGTCAGATGTGGAAAGATCTACTGCACTGGAGGGGAGCTTTC
CTCTCTCCTTGGAGAAGACAAGACTTATCACCTTAAGGATCCCCAGAAGAATGCTACTGTCAAATGCA
AAACTATTTTTTTATACCATGATTCTACAGACATTGGCCTGGTGGCGTCAGGAACAAAATGTGGAGAG
GGAATGGTATGCAACAATGGTGAATGTCTAAACATGGAAAAGGTCTATATCTCAACCAATTGCCCCTC
TCAGTGCAATGAAAATCCTGTAGATGGCCACGGACTCCAGTGCCACTGTGAGGAAGGACAGGCACCTG
TAGCCTGTGAAGAAACCTTACATGTTACCAGTATCACCATCTTGGTTGTTGTGCTTGTCCTGGTTATT
GTCGGTATCGGAGTTCTTATACTATTAGTTCGTTACCGAAAATGTATCAAGTTGAAGCAAGTTCAGAG
CCCACCTACAGAAACCCTGGGAGTGGAGAACAAAGGATACTTTGGTGATGAGCAGCAGATAAGGACTG
AGCCAATCCTGCCAGAAATTCATTTCCTAAATAAACCTGCAAGTAAAGATTCAAGAGGAATCGCAGAT
CCCAATCAAAGTGCCAAGTGGTAG

| | | | |
|---|---|---|---|
| NOV22h, SNP13376586 of | SEQ ID NO: 304 | 755 aa | MW at 85810.5kD |
| CG51983-05, Protein | SNP Pos: 42 | | SNP Change: Asp to Gly |
| Sequence | | | |

MLPGCIFLMILLIPQVKEKFILGVEGQQLVRPKKLPLIQKRGTGHTHDDDILKTYEEELLYEIKLNRK
TLVLHLLRSREFLGSNYSETFYSMKGEAFTRHPQIMEHCYYKGNILNEKNSVASISTCDGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNMIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGMQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCAEGECCESCQIKKAGSICRPAKDECDFPEMCTGH
SPACPKDQFRVNGFPCKNSEGYCFMGKCPTREDQCSELFDDEAIESHDICYKMNTKGNKFGYCKNKEN
RFLPCEEKDVRCGKIYCTGGELSSLLGEDKTYHLKDPQKNATVKCKTIFLYHDSTDIGLVASGTKCGE
GMVCNNGECLNNEKVYISTNCPSQCNENPVDGHGLQCHCEEGQAPVACEETLHVTSITILVVVLVLVI
VGIGVLILLVRYRKCIKLKQVQSPPTETLGVENKGYFGDEQQIRTEPILPEIHFLNKPASKDSRGIAD
PNQSAKW

A ClustalW comparison of the above protein sequences yields the following sequence alignment shown in Table 22B.

TABLE 22B
Comparison of the NOV22 protein sequences.

NOV22a MLPGCIFLMILLIPQVKEKFILGVEGQQLVRPKKLPLIQKRDTGHTHDDDILKTYEEELL
NOV22b MLPGCIFLMILLIPQVKEKFILGVEGOQLVRPKKLPLIQKRDTGHTHDDDIK-TYEEELL
NOV22c MLPGCIFLMILLIPQVKEKFILGVEGQQLVRPKKLPLIQKRDTGHTHDDDILKTYEEELL
NOV22d MLPGCIFLMILLIPOVKEKFILGVEGQQLVRPKKLPLIQKRDTGHTHDDDIK-TYEEELL
NOV22e MLPGCIFLMILLIPQVKEKFILGVEGQQLVRPKKLPLIQKRDTGHTHDDDILKTYEEELL
NOV22f MLPGCIFLMILLIPQVKEKFILGVEGQQLVRPKKLPLIQKRDTGHTHDDDILKTYEEELL
NOV22a YEIKLNRKTLVLHLLRSR-EFLGSNYSETFYSMKGEAFTRHPQIMEHCYYKGNILNEKNS
NOV22b YEIKLNRKTLVLHLLRSRREFLGSNYSETFYSMKGEAFTRHPQIMDHCFYQGSIVHEYDS
NOV22c YEIKLNRKTLVLHLLRSR-EFLGSNYSETFYSMKGGAFTRHPQIMDHCFYQGSIVHEYDS
NOV22d YEIKLNRKTLVLHLLRSRREFLGSNYSETFYSMKGEAFTRHPQIMDHCFYQGSIVHEYDS
NOV22e YEIKLNRKTLVLHLLRSR-EFLGSNYSETFYSMKGGAFTRHPQIMDHCFYQGSIVHEYDS
NOV22f YEIKLNRKTLVLHLLRSR-EFLGSNYSETFYSMKGEAFTRHPQIMDHCFYQGSIVHEYDS
NOV22a VASISTCDGLRGFFRINDQRYLIEPVKYSDEGEHLVFKYNLRVPYGANYSCTELNFTRKT
NOV22b AASISTCNGLRGFFRINDQRYLIEPVKYSDEGEHLVFYNLKRVPYGANYSCTELNFTRKT
NOV22c AASISTCNGLRGFFRINDQRYLIEPVKYSDEGEHLVFYNLKRVPYGANYSCTELNFTRKT
NOV22d AASISTCNGLRGFFRINDQRYLIEPVKYSDEGEHLVFYNLKRVPYGANYSCTELNFTRKT
NOV22e AASISTCNGLRGFFRINDQRYLIEPVKYSDEGEHLVFYNLKRVPYGANYSCTELNFTRKT
NOV22f AASISTCNGLRGFFRINDQRYLIEPVKYSDEGEHLVFYNLKRVPYGANYSCTELNFTRKT
NOV22a VPGDNESEEDSKIKG-IHDEKYVELFIVADDTVYRRNGHPHNKLRNRIWGMVNFVNMIYK
NOV22b VPGDNESEEDSKIKQGIHDEKYVELFIVADDTVYRRNGHPHNKLRNRIWGMVNFVNMIYK
NOV22c VPGDNESEEDSKIKG-IHDEKYVELFIVADDTVYRRNGHPHNKLRNRIWGMVNFVNMIYK
NOV22d VPGDNESEEDSKIKQGIHDEKYVELFIVADDTVYRRNGHPHNKLRNRIWGMVNFVNMIYK
NOV22e VPGDNESEEDSKIKG-IHDEKYVELFIVADDTVYRRNGHPHNKLRNRIWGMVNFVNMIYK
NOV22f VPGDNESEEDSKIKG-IHDEKYVELFIVADDTVYRRNGHPHNKLRNRIWGMVNFVNMIYK
NOV22a TLNIHVTLVGIEIWTHEDKIELYSNIETTLLRFSFWQEKILKTRKDFDHVVLLSGKWLYS
NOV22b TLNIHVTLVGIEIWTHEDKIELYSNIETTLLRFSFWQEKILKTRKDFDHVVLLSGKWLYS
NOV22c TLNIHVTLVGIEIWTHEDKIELYSNIETTLLRFSFWQEKILKTRKDFDHVVLLSGKWLYS
NOV22d TLNIHVTLVGIEIWTHEDKIELYSNIETTLLRFSFWQEKILKTRKDFDHVVLLSGKWLYS
NOV22e TLNIHVTLVGIEIWTHEDKIELYSNIETTLLRFSFWQEKILKTRKDFDHVVLLSGKWLYS
NOV22f TLNIHVTLVGIEIWTHEDKIELYSNIETTLLRFSFWQEKILKTRKDFDHVVLLSGKWLYS
NOV22a HVQGISYPGGMCLPYYSTSIIKDLLPDTNIIANRMAHQLGHNLGMQHDEFPCTCPSGKCV
NOV22b HVQGISYPGGMCLPYYSTSIIKDLLPDTNIIANRMAHQLGHNLGMQHDEFPCTCPSGKCV
NOV22c HVQGISYPGGMCLPYYSTSIIKDLLPDTNIIANRMAHQLGHNLGMQHDEFPCTCPSGKCV
NOV22d HVQGISYPGGMCLPYYSTSIIKDLLPDTNIEANRMAHQLGHNLGMQHDEFPCTCPSGKCV
NOV22e HVQGISYPGGMCLPYYSTSIIKDLLPDTNIIANRMAHQLGHNLGMQHDEFPCTCPSGKCV
NOV22f HVQGISYPGGMCLPYYSTSIIKDLLPDTNIIANRMAHQLGHNLGMQHDEFPCTCPSGKCV
NOV22a MDSDGSIPALKFSKCSQNQYHQYLKDYKPTCMLNIPFPYNFHDFQFCGNKKLDEGEECDC
NOV22b MDSDGSIPALKFSKCSQNQYHQYLKDYKPTCMLNIPFPYNFHDFQFCGNKKLDEGEECDC
NOV22c MDSDGSIPALKFSKCSQNQYHQYLKDYKPTCMLNIPFPYNFHDFQFCGNKKLDEGEECDC
NOV22d MDSDGSIPALKFSKCSQNQYHQYLKDYKPTCMLNIPFPYNFHDFQFCGNKKLDEGEECDC
NOV22e MDSDGSIPALKFSKCSQNQYHQYLKDYKPTCMLNIPFPYNFHDFQFCGNKKLDEGEECDC
NOV22f MDSDGSIPALKFSKCSQNQYHQYLKDYKPTCMLNIPFPYNFHDFQFCGNKKLDEGEECDC
NOV22a GPAQECTNPCCDAHTCVLKPGFTCAEGECCESCQIKKAGSICRPAKDECDFPEMCTGHSP
NOV22b GPAQECTNPCCDAHTCVLKPGFTCAEGECCESCQIKKAGSICRPAKDECDFPEMCTGHSP
NOV22c GPAQECTNPCCDAHTCVLKPGFTCAEGECCESCQIKKAGSICRPAKDECDFPEMCTGHSP
NOV22d GPAQECTNPCCDAHTCVLKPGFTCAEGECCESCQIKKAGSICRPAKDECDFPENCTGHSP
NOV22e GPAQECTNPCCDAHTCVLKPGFTCABGECCESCQIKKAGSICRPAKDECDFPEMCTGHSP
NOV22f GPAQECTNPCCDAHTCVLKPGFTCAEGECCESCQIKKAGSICRPAKDECDFPEMCTGHSP
NOV22a ACPKDQFRVNGFPCKNSEGYCFMGKCPTREDQCSELFDDEAIESHDICYKMNTKGNKFGY
NOV22b ACPKDQFRVNGFPCKNSEGYCFMGKCPTREDQCSELFDDEAIESHDICYKMNTKGNKFGY
NOV22c ACPKDQFRVNGFPCKNSEGYCFMGKCPTREDQCSELFDDDAIESHDICYKMNTKGNKFGY
NOV22d ACPKDQFRVNGFPCKNSEGYCFMGKCPTREDQCSELFDDEAIESHDICYKMNTKGNKFGY
NOV22e ACPKDQFRVNGFPCKNSEGYCFMGKCPTREDQCSELFDDDAIESHDICYKMNTKGNKFGY
NOV22f ACPKDQFRVNGFPCKNSEGYCFMGKCPTREDQCSELFDDEAIESHDICYKMNTKGNKFGY
NOV22a CKNKENRFLPCEEKDVRCGKIYCTGGELSSLLGEDKTYHLKDPQKNATVKCKTIFLYHDS
NOV22b CKNKENRFLPCEEKDVRCGKIYCTGGELSSLLGEDKTYHLKDPQKNATVKCKTIFLYHDS
NOV22c CKNKENRFLPCEEKDVRCGKIYCTGGELSSLLGEDKTYHLKDPQKNATVKCKTIFLYHDS
NOV22d CKNKENRFLPCEEKDVRCGKIYCTGGELSSLLGEDKTYHLKDPQKNATVKCKTIFLYHDS
NOV22e CKNKENRFLPCEEKDVRCGKIYCTGGELSSLLGEDKTYHLKDPQKNATVKCKTIFLYHDS
NOV22f CKNKENRFLPCEEKDVRCGKIYCTGGELSSLLGEDKTYHLKDPQKNATVKCKTIFLYHDS
NOV22a TDIGLVASGTKCGEGMVCNNGECLNMEKVYISTNCPSQGNENPVDGHGLQCHCEEGQAPV
NOV22b TDIGLVASGTKCGEGMVCNNGECLNMEKVYISTNCPSQCNENPVDGHGLQCHCEEGQAPV
NOV22c TDIGLVASGTKCGEGMVCNNGECLNMEKVYISTNCPSQcNENPVDGHGLQCHCEEGQAPV
NOV22d TDIGLVASGTKCGEGMVCNNGECLNMEKVYISTNCPSQCNENPVDGHGLQCHCEEGQAPV
NOV22e TDIGLVASGTKCGEGMVCNNGECLNMEKVYISTNCPSQCNENPVDGHGLQCHCEEGQAPV
NOV22f TDIGLVASGTKCGEGMVD---------------------------GHGLQCHCEEGQAPV
NOV22a ACEETLHVTSITILVVVLVLVIVGIGVLILLVRYRKCIKLKQVQSPPTETLGVENKGYFG
NOV22b ACEETLHVTSITILVVVLVLVIVGIGVLILLVRYRKCIKLKQVQSPPTETLGVENKGYFG
NOV22c ACEETLHVTNITILVVVLVLVIVGIGVLILLVRYRKCIKLKQVQSPPTETLGVENKGYFG
NOV22d ACEETLHVTSITILVVVLVLVIVGIGVLILLVRYRKCIKLKQVQSPPTETLGVENKGYFG
NOV22e ACEETLHVTNITILVVVLVLVIVGIGVLILLVRYRKCIKLKQVQSPPTETLGVENKGYFG
NOV22f ACEETLHVTNITILVVVLVLVIVGIGVLILLVRYRKCIKLKQVQSPPTETLGVENKGYFG
NOV22a DEQQIRTEPILPEIHFLN----------------------KPASKDSRGIADPNQSAKW
NOV22b DEQQIRTEPILPEIHFLNQRTPESLESLPTSFSSPHYITLKPASKDSRGIADPNQSAKW
NOV22c DEQQIRTEPILPEIHFLN----------------------KPASKDSRGIADPNQSAK-
NOV22d DEQQIRTEPILPEIHFLN----------------------KPASKDSRGIADPNQSAKW
NOV22e DEQQIRTEPILPEIHFLN----------------------KPASKDSRGIADPNQSAK-
NOV22f DEQQIRTEPILPEIHFLN-RTPESLESLPTSFSSPHYITLKPASKDSRGIADPNQSAK-
NOV22a (SEQ ID NO: 290)
NOV22b (SEQ ID NO: 292)
NOV22c (SEQ ID NO: 294)
NOV22d (SEQ ID NO: 296)
NOV22e (SEQ ID NO: 298)
NOV22f (SEQ ID NO: 300)

Further analysis of the NOV22a protein yielded the following properties shown in Table 22C.

TABLE 22C
Protein Sequence Properties NOV22a

SignalP analysis: Cleavage site between residues 19 and 20
PSORT II analysis:

| PSG: | a new signal peptide prediction method |
|---|---|
| | N-region: length 0; pos.chg 0; neg.chg 0 |
| | H-region: length 16; peak value 12.97 |
| | PSG score: 8.57 |
| GvH: | von Heijne's method for signal seq. recognition |
| | GvH score (threshold: −2.1): −5.58 |
| | possible cleavage site: between 18 and 19 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 2 | |
| | INTEGRAL Likelihood = −2.18 | Transmembrane 1-17 |
| | INTEGRAL Likelihood = −18.52 | Transmembrane 671-687 |

| | PERIPHERAL Likelihood = 4.35 (at 235) |
|---|---|
| | ALOM score: —18.52 (number of TMSs: 2) |
| MTOP: | Prediction of membrane topology (Hartmann et al.) |
| | Center position for calculation: 8 |
| | Charge difference: 0.0 C( 1.0) - N( 1.0) |
| | N >= C: N-terminal side will be inside |

>>> membrane topology: type 3a
MITDISC: discrimination of mitochondrial targeting seq

| | | | |
|---|---|---|---|
| R content: | 0 | Hyd Moment(75): | 3.80 |
| Hyd Moment(95): | 1.48 | G content: | 1 |
| D/E content: | 1 | S/T content: | 0 |
| Score: −6.33 | | | |

Gavel: prediction of cleavage sites for mitochondrial preseq

cleavage site motif not found

NUCDISC: discrimination of nuclear localization signals

pat4: RPKK (4) at 31
pat7: none
bipartite: none
content of basic residues: 11.5%
NLS Score: −0.22

KDEL: ER retention motif in the C-terminus: none
ER Membrane Retention Signals:

KKXX-like motif in the C-terminus: QSAK
SKL: peroxisomal targeting signal in the C-terminus: none

PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:

Bacterial regulatory proteins, gntR family signature (PS00043):

*** found ***

EEELLYEIKLNRKTLVLHLLRS at 56

NNCN: Reinhardt's method for Cytoplasmic/Nuclear discrimination

Prediction: nuclear
Reliability: 55.5

COIL: Lupas's algorithm to detect coiled-coil regions

total: 0 residues

--------------------------
Final Results (k = 9/23)

```
34.8%: nuclear
30.4%: endoplasmic reticulum
21.7%: mitochondrial
  4.3%: vesicles of secretory system
  4.3%: cytoplasmic
  4.3%: peroxisomal
```

>> prediction for CG51983-05 is nuc (k = 23)

A search of the NOV22a protein against the Geneseq database, a proprietary database that contains sequences published in patents and patent publication, yielded several homologous proteins shown in Table 22D.

TABLE 22D
Geneseq Results for NOV22a

| | | NOV22a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB47567 | Protease PRTS-9 - Homo sapiens, | 1 . . . 754 | 749/776 (96%) | 0.0 |
| | 776 aa. [WO200171004-A2, 27- | 1 . . . 776 | 751/776 (96%) | |
| | SEP-2001] | | | |
| AAU77409 | Human NOV4b protein, | 1 . . . 755 | 754/780 (96%) | 0.0 |
| | homologue of ADAM proteins - | 1 . . . 779 | 754/780 (96%) | |
| | Homo sapiens, 779 aa. | | | |
| | [WO200206329-A2, 24-JAN- | | | |
| | 2002] | | | |
| AAU77408 | Human NOV4a protein, | 1 . . . 755 | 744/779 (95%) | 0.0 |
| | homologue of ADAM proteins - | 1 . . . 778 | 752/779 (96%) | |
| | Homo sapiens, 778 aa. | | | |
| | [WO200206329-A2, 24-JAN- | | | |
| | 2002] | | | |
| AAU16950 | Human novel secreted protein, | 1 . . . 677 | 664/677 (98%) | 0.0 |
| | SEQ ID 191 - Homo sapiens, 695 | 17 . . . 693 | 675/677 (99%) | |
| | aa. [WO200155441-A2, 02-AUG- | | | |
| | 2001] | | | |
| AAB64744 | Gene 16 human secreted protein | 336 . . . 710 | 353/375 (94%) | 0.0 |
| | homologous amino acid sequence | 1 . . . 375 | 364/375 (96%) | |
| | #138 - Macaca fascicularis, 375 | | | |
| | aa. [WO200077237-A1, 21-DEC- | | | |
| | 2000] | | | |

In a BLAST search of public sequence databases, the NOV22a protein was found to have homology to the proteins shown in the BLASTP data in Table 22E.

TABLE 22E
Public BLASTP Results for NOV22a

| | | NOV22a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H2U9 | ADAM 7 precursor (A disintegrin | 1 . . . 754 | 741/754 (98%) | 0.0 |
| | and metalloproteinase domain 7) | 1 . . . 754 | 751/754 (99%) | |
| | (Sperm maturation-related | | | |
| | glycoprotein GP-83) - Homo | | | |
| | sapiens (Human), 754 aa. | | | |
| Q28475 | ADAM 7 precursor (A disintegrin | 1 . . . 754 | 695/776 (89%) | 0.0 |
| | and metalloproteinase domain 7) | 1 . . . 776 | 726/776 (92%) | |
| | (Epididymal apical protein I) (EAP | | | |
| | I) - Macaca fascicularis (Crab | | | |
| | eating macaque) (Cynomolgus | | | |
| | monkey), 776 aa. | | | |
| AAH43207 | Hypothetical protein - Homo | 212 . . . 736 | 524/525 (99%) | 0.0 |
| | sapiens (Human), 561 aa | 29 . . . 553 | 525/525 (99%) | |
| | (fragment). | | | |
| O35227 | ADAM 7 precursor (A disintegrin | 1 . . . 752 | 507/773 (65%) | 0.0 |
| | and metalloproteinase domain 7) - | 1 . . . 771 | 620/773 (79%) | |
| | Mus musculus (Mouse), 788 aa. | | | |
| Q63180 | ADAM 7 precursor (A disintegrin | 1 . . . 752 | 505/773 (65%) | 0.0 |
| | and metalloproteinase domain 7) | 1 . . . 772 | 614/773 (79%) | |
| | (Epididymal apical protein I) (EAP | | | |
| | I) - Rattus norvegicus (Rat), 789 | | | |
| | aa. | | | |

Pam analysis indicates that the NOV22a protein contains the domains shown in the Table 22F.

TABLE 22F
Domain Analysis of NOV22a

| | NOV22a | Identities/ | |
|---|---|---|---|
| | Match | Similarities for | |
| Pfam Domain | Region | the Matched Region | Expect Value |
| Pep_M12B_propep | 73 . . . 188 | 38/119 (32%) | 6.7e−45 |
| | | 100/119 (84%) | |
| Reprolysin | 199 . . . 394 | 88/203 (43%) | 5.5e−104 |
| | | 173/203 (85%) | |
| disintegrin | 411 . . . 486 | 41/76 (54%) | 1.6e−25 |
| | | 53/76 (70%) | |

### Example 23

The NOV23 clone was analyzed, and the nucleotide and encoded polypeptide sequences are shown in Table 23A.

TABLE 23A
NOV23 Sequence Analysis

| | | |
|---|---|---|
| NOV23a, CG53390-02 | SEQ ID NO: 305 | 994 bp |
| DNA Sequence | ORF Start: ATG at 27 | ORF Stop: TGA at 969 |

TGCAGCTAAAGTGCATTGTGTAAAACATGGGGGATGTGAATCAGTCGGTGGCCTCAGACTTCATTCTG
GTGGGCCTCTTCAGTCACTCAGGATCACGCCAGCTCCTCTTCTCCCTGGTGGCTGTCATGTTTGTCAT
AGGCCTTCTGGGCAACACCGTTCTTCTCTTCTTGATCCGTGTGGACTCCCGGCTCCATACACCCATGT
ACTTCCTGCTCAGCCAGCTCTCCCTGTTTGACATTGGCTGTCCCATGGTCACCATCCCCAAGATGGCA
TCAGACTTTCTGCGGGGAGAAGGTGCCACCTCCTATGGAGGTGGTGCAGCTCAAATATTCTTCCTCAC
ACTGATGGGTGTGGCTGAGGGCGTCCTGTTGGTCCTCATGTCTTATGACCGTTATGTTGCTGTGTGCC
AGCCCCTGCAGTATCCTGTACTTATGAGACGCCAGGTATGTCTGCTGATGATGGGCTCCTCCTGGGTG
GTAGGTGTGCTCAACGCCTCCATCCAGACCTCCATCACCCTGCATTTTCCCTACTGTGCCTCCCGTAT
TGTGGATCACTTCTTCTGTGAGGTGCCAGCCCTACTGAAGCTCTCCTGTGCAGATACCTGTGCCTACG
AGATGGCGCTGTCCACCTCAGGGGTGCTGATCCTAATGCTCCCTCTTTCCCTCATCGCCACCTCCTAC
GGCCACGTGTTGCAGGCTGTTCTAAGCATGCGCTCAGAGGAGGCCAGACACAAGGCTGTCACCACCTG
CTCCTCGCACATCACGGTAGTGGGGCTCTTTTATGGTGCCGCCGTGTTCATGTACATGGTGCCTTGCG
CCTACCACAGTCCACAGCAGGATAACGTGGTTTCCCTCTTCTATAGCCTTGTCACCCCTACACTCAAC
CCCCTTATCTACAGTCTGAGGAATCCGGAGGTGTGGATGGCTTTGGTCAAAGTGCTTAGCAGAGCTGG
ACTCAGGCAAATGTGCTGACTACATAGAAACTGCTGGTGAGA

| | | | |
|---|---|---|---|
| NOV23a, CG53390-02 | SEQ ID NO: 306 | 314 aa | MW at 34443.4kD |
| Protein Sequence | | | |

MGDVNQSVASDFILVGLFSHSGSRQLLFSLVAVMFVIGLLGNTVLLFLIRVDSRLHTPMYFLLSQLSL
FDIGCPMVTIPKMASDFLRGEGATSYGGGAAQIFFLTLMGVAEGVLLVLMSYDRYVAVCQPLQYPVLM
RRQVCLLMMGSSWVVGVLNASIQTSITLHFPYCASRIVDHFFCEVPALLKLSCADTCAYEMALSTSGV
LILMLPLSLIATSYGHVLQAVLSMRSEEARHKAVTTCSSHITVVGLFYGAAVFMYMVPCAYHSPQQDN
VVSLFYSLVTPTLNPLIYSLRNPEVWMALVKVLSRAGLRQMC

| | | |
|---|---|---|
| NOV23b, CG53390-01 | SEQ ID NO: 307 | 994 bp |
| DNA Sequence | ORF Start: ATG at 27 | ORF Stop: TGA at 969 |

TGCAGCTAAAGTGCATTGTGTAAAACATGGGGGATGTGAATCAGTCGGTGGCCTCAGACTTCATTCTG
GTGGGCCTCTTCAGTCACTCAGGATCACGCCAGCTCCTCTTCTCCCTGGTGGCTGTCATGTTTGTCAT
AGGCCTTCTGGGCAACACCGTTCTTCTCTTCTTGATCCGTGTGGACTCCCGGCTCCATACACCCATGT
ACTTCCTGCTCAGCCAGCTCTCCCTGTTTGACATTGGCTGTCCCATGGTCACCATCCCCAAGATGGCA
TCAGACTTTCTGCGGGGAGAAGGTGCCACCTCCTATGGAGGTGGTGCAGCTCAAATATTCTTCCTCAC
ACTGATGGGTGTGGCTGAGGGCGTCCTGTTGGTCCTCATGTCTTATGACCGTTATGTTGCTGTGTGCC
AGCCCCTGCAGTATCCTGTACTTATGAGACGCCAGGTATGTCTGCTGATGATGGGCTCCTCCTGGGTG
GTAGGTGTGCTCAACGCCTCCATCCAGACCTCCATCACCCTGCATTTTCCCTACTGTGCCTCCCGTAT
TGTGGATCACTTCTTCTGTGAGGTGCCAGCCCTACTGAAGCTCTCCTGTGCAGATACCTGTGCCTACG
AGATGGCGCTGTCCACCTCAGGGGTGCTGATCCTAATGCTCCCTCTTTCCCTCATCGCCACCTCCTAC
GGCCACGTGTTGCAGGCTGTTCTAAGCATGCGCTCAGAGGAGGCCAGACACAAGGCTGTCACCACCTG
CTCCTCGCACATCACGGTAGTGGGGCTCTTTTATGGTGCCGCCGTGTTCATGTACATGGTGCCTTGCG
CCTACCACAGTCCACAGCAGGATAACGTGGTTTCCCTCTTCTATAGCCTTGTCACCCCTACACTCAAC
CCCCTTATCTACAGTCTGAGGAATCCGGAGGTGTGGATGGCTTTGGTCAAAGTGCTTAGCAGAGCTGG
ACTCAGGCAAATGTGCTGACTACATAGAAACTGCTGGTGAGA

| | | | |
|---|---|---|---|
| NOV23b, CG53390-01 | SEQ ID NO: 308 | 314 aa | MW at 34443.4kD |
| Protein Sequence | | | |

MGDVNQSVASDFILVGLFSHSGSRQLLFSLVAVMFVIGLLGNTVLLFLIRVDSRLHTPMYFLLSQLSL
FDIGCPMVTIPKMASDFLRGEGATSYGGGAAQIFFLTLMGVAEGVLLVLMSYDRYVAVCQPLQYPVLM
RRQVCLLMMGSSWVVGVLNASIQTSITLHFPYCASRIVDHFFCEVPALLKLSCADTCAYEMALSTSGV
LILMLPLSLIATSYGHVLQAVLSMRSEEARHKAVTTCSSHITVVGLFYGAAVFMYMVPCAYHSPQQDN
VVSLFYSLVTPTLNPLIYSLRNPEVWMALVKVLSPAGLRQMC

| | | |
|---|---|---|
| NOV23c, CG53390-03 | SEQ ID NO: 309 | 977 bp |
| DNA Sequence | ORF Start: ATG at 16 | ORF Stop: TGA at 958 |

TGCATTGTGTAAAACATGGGGGATGTGAATCAGTCGGTGGCCTCAGACTTCATTCTGGTGGGCCTCTT
CAGTCACTCAGGATCACGCCAGCTCCTCTTCTCCCTGGTGGCTGTCATGTTTGTCATAGGCCTTCTGG
GCAACACCGTTCTTCTCTTCTTGATCCGTGTGGACTCCCGGCTCCATACACCCATGTACTTCCTGCTC
AGCCAGCTCTCCCTGTTTGACATTGGCTGTCCCATGGTCACCATCCCCAAGATGGCATCAGACTTTCT
GCGGGGAGAAGGTGCCACCTCCTATGGAGGTGGTGCAGCTCAAATATTCTTCCTCACACTGATGGGTG
TGGCTGAGGGCGTCCTGTTGGTCCTCATGTCTTATGACCGTTATGTTGCTGTGTGCCAGCCCCTGCAG
TATCCTGTACTTATGAGACGCCAGGTATGTCTGCTGATGATGGGCTCCTCCTGGGTGGTAGGTGTGCT
CAACGCCTCCATCCAGACCTCCATCACCCTGCATTTTCCCTACTGTGCCTCCCGTATTGTGGATCACT
TCTTCTGTGAGGTGCCAGCCCTACTGAAGCTCTCCTGTGCAGATACCTGTGCCTACGAGATGGCGCTG
TCCACCTCAGGGGTGCTGATCCTAATGCTCCCTCTTTCCGTCATCGCCACCTCCTACGGCCACGTGTT
GCAGGCTGTTCTAAGCATGCGCTCAGAGGAGGCCAGACACAAGGCTGTCACCACCTGCTCCTCGCACA
TCACGGTAGTGGGGCTCTTTTATGGTGCCGCCGTGTTCATGTACATGGTGCCTTGCGCCTACCACAGT
CCACAGCAGGATAACGTGGTTTCCCTCTTCTATAGCCTTGTCACCCCTACACTCAACCCCCTTATCTA
CAGTCTGAGGAATCCGGAGGTGTGGATGGCTTTGGTCAAAGTGCTTAGCAGAGCTGGACTCAGGCAAA
TGTGCTGACTACATAGAAACTGCTG

| | | | |
|---|---|---|---|
| NOV23c, CG53390-03 | SEQ ID NO: 310 | 314 aa | MW at 34443.4kD |
| Protein Sequence | | | |

MGDVNQSVASDFILVGLFSHSGSRQLLFSLVAVMFVIGLLGNTVLLFLIRVDSRLHTPMYFLLSQLSL
FDIGCPMVTIPKMASDFLRGEGATSYGGGAAQIFFLTLMGVAEGVLLVLMSYDRYVAVCQPLQYPVLM
RRQVCLLMMGSSWVVGVLNASIQTSITLHFPYCASRIVDHFFCEVPALLKLSCADTCAYEMALSTSGV
LILMLPLSLIATSYGHVLQAVLSMRSEEARHKAVTTCSSHITVVGLFYGAAVFMYMVPCAYHSPQQDN
VVSLFYSLVTPTLNPLIYSLRNPEVWMALVKVLSRAGLRQMC

A ClustalW comparison of the above protein sequences yields the following sequence alignment shown in Table 23B.

TABLE 23B
Comparison of the NOV23 protein sequences.

NOV23a MGDVNQSVASDFILVGLFSHSGSRQLLFSLVAVMFVIGLLGNTVLLFLIRVDSRLHTPMY
NOV23b MGDVNQSVASDFILVGLFSHSGSRQLLFSLVAVMFVIGLLGNTVLLFLIRVDSRLHTPMY
NOV23c MGDVNQSVASDFILVGLFSHSGSRQLLFSLVAVMFVIGLLGNTVLLFLIRVDSRLHTPMY
NOV23a FLLSQLSLFDIGCPMVTIPKMASDFLRGEGATSYGGGAAQIFFLTLMGVAEGVLLVLMSY
NOV23b FLLSQLSLFDIGCPMVTIPKMASDFLRGEGATSYGGGAAQIFFLTLMGVAEGVLLVLMSY
NOV23c FLLSQLSLFDIGCPMVTIPKMASDFLRGEGATSYGGGAAQIFFLTLMGVAEGVLLVLMSY
NOV23a DRYVAVCQPLQYPVLMRRQVCLLMMGSSWVVGVLNASIQTSITLHFPYCASRIVDHFFCE
NOV23b DRYVAVCQPLQYPVLMRRQVCLLMMGSSWVVGVLNASIQTSITLHFPYCASRIVDHFFCE
NOV23c DRYVAVCQPLQYPVLNRRQVCLLMMGSSWVVGVLNASIQTSITLHFPYCASRIVDHFFCE
NOV23a VPALLKLSCADTCAYEMALSTSGVLILMLPLSLIATSYGHVLQAVLSMRSEEARHKAVTT
NOV23b VPALLKLSCADTCAYEMALSTSGVLILMLPLSLIATSYGHVLQAVLSMRSEEARHKAVTT
NOV23c VPALLKLSCADTCAYEMALSTSGVLILMLPLSLIATSYGHVLQAVLSMRSEEARHKAVTT
NOV23a CSSHITVVGLFYGAAVFMYMVPCAYHSPQQDNVVSLFYSLVTPTLNPLIYSLRNPEVWMA
NOV23b CSSHITVVGLFYGAAVFMYMVPCAYHSPQQDNVVSLFYSLVTPTLNPLIYSLRNPEVWMA
NOV23c CSSHITVVGLFYGAAVFMYMVPCAYHSPQQDNVVSLFYSLVTPTLNPLIYSLRNPEVWMA
NOV23a LVKVLSRAGLRQMC
NOV23b LVKVLSRAGLRQMC
NOV23c LVKVLSRAGLRQMC
NOV23a (SEQ ID NO: 306)
NOV23b (SEQ ID NO: 308)
NOV23c (SEQ ID NO: 310)

Further analysis of the NOV23a protein yielded the following properties shown in Table 23C.

TABLE 23C
Protein Sequence Properties NOV23a

| | |
|---|---|
| SignalP analysis: | Cleavage site between residues 42 and 43 |
| PSORT II analysis: | |

| | |
|---|---|
| PSG: | a new signal peptide prediction method |
| | N-region: length 11; pos.chg 0; neg.chg 2 |
| | H-region: length 12; peak value 0.00 |
| | PSG score: −4.40 |
| GvH: | von Heijne's method for signal seq. recognition |
| | GvH score (threshold: −2.1): −1.30 |
| | possible cleavage site: between 41 and 42 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood = −8.60 | Transmembrane 33-49 |
| | INTEGRAL | Likelihood = −7.17 | Transmembrane 101-117 |
| | INTEGRAL | Likelihood = −1.38 | Transmembrane 140-156 |
| | INTEGRAL | Likelihood = −4.09 | Transmembrane 198-214 |
| | INTEGRAL | Likelihood = −3.77 | Transmembrane 245-261 |

| | |
|---|---|
| PERIPHERAL | Likelihood = 1.38 (at 62) |

| | |
|---|---|
| | ALOM score: −8.60 (number of TMSs: 5) |
| MTOP: | Prediction of membrane topology (Hartmann et al.) |
| | Center position for calculation: 40 |
| | Charge difference: 1.0 C(1.5) − N(0.5) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 0.74 |
| Hyd Moment(95): | 7.13 | G content: | 1 |
| D/E content: | 2 | S/T content: | 2 |

Score: −7.54

Gavel: prediction of cleavage sites for mitochondrial preseq

cleavage site motif not found

NUCDISC: discrimination of nuclear localization signals

pat4: none
pat7: none
bipartite: none
content of basic residues: 5.4%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
11.1%: Golgi
11.1%: vacuolar
11.1%: vesicles of secretory system
11.1%: mitochondrial

>> prediction for CG53390-02 is end (k = 9)

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85207 | G-coupled olfactory receptor #68 - | 1 . . . 314 | 314/314 (100%) | e−179 |
| | Homo sapiens, 314 aa. | 1 . . . 314 | 314/314 (100%) | |
| | [WO200198526-A2, 27-DEC- | | | |
| | 2001] | | | |
| AAU95551 | Human olfactory and pheromone | 1 . . . 314 | 314/314 (100%) | e−179 |
| | G protein-coupled receptor #38 - | 1 . . . 314 | 314/314 (100%) | |
| | Homo sapiens, 314 aa. | | | |
| | [WO200224726-A2, 28-MAR- | | | |
| | 2002] | | | |
| ABJ04025 | Human G-protein coupled | 1 . . . 314 | 314/314 (100%) | e−179 |
| | receptor SEQ ID NO: 116 - | 1 . . . 314 | 314/314 (100%) | |
| | Homo sapiens, 314 aa. | | | |
| | [WO200255558-A2, 18-JUL- | | | |
| | 2002] | | | |
| ABP95895 | Human GPCR polypeptide SEQ | 1 . . . 314 | 314/314 (100%) | e−179 |
| | ID NO 600 - Homo sapiens, 314 | 1 . . . 314 | 314/314 (100%) | |
| | aa. [WO200216548-A2, 28-FEB- | | | |
| | 2002] | | | |
| AAG71902 | Human olfactory receptor | 1 . . . 314 | 314/314 (100%) | e−179 |
| | polypeptide, SEQ ID NO: 1583 - | 1 . . . 314 | 314/314 (100%) | |
| | Homo sapiens, 314 aa. | | | |
| | [WO200127158-A2, 19-APR- | | | |
| | 2001] | | | |

In a BLAST search of public sequence databases, the NOV23a protein was found to have homology to the proteins shown in the BLASTP data in Table 23E.

TABLE 23E
Public BLASTP Results for NOV23a

| | | NOV23a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NG97 | Seven transmembrane helix | 1 . . . 314 | 314/314 (100%) | e−179 |
| | receptor - Homo sapiens | 1 . . . 314 | 314/314 (100%) | |
| | (Human), 314 aa. | | | |
| Q8VFG8 | Olfactory receptor MOR282-1 - | 1 . . . 301 | 225/301 (74%) | e−126 |
| | Mus musculus (Mouse), 308 aa. | 1 . . . 301 | 255/301 (83%) | |
| Q96R25 | Olfactory receptor - Homo | 68 . . . 284 | 217/217 (100%) | e−122 |
| | sapiens (Human), 217 aa | 1 . . . 217 | 217/217 (100%) | |
| | (fragment). | | | |
| Q8VGD8 | Olfactory receptor MOR281-1 - | 5 . . . 312 | 173/308 (56%) | 2e−97 |
| | Mus musculus (Mouse), 315 aa. | 7 . . . 313 | 229/308 (74%) | |
| Q8VGD7 | Olfactory receptor MOR277-1 - | 5 . . . 307 | 170/304 (55%) | 3e−91 |
| | Mus musculus (Mouse), 317 aa. | 6 . . . 308 | 218/304 (70%) | |

PFam analysis indicates that the NOV23a protein contains the domains shown in the Table 23F.

TABLE 23F
Domain Analysis of NOV23a

| | | Identities/ | |
|---|---|---|---|
| | NOV23a Match | Similarities for | |
| Pfam Domain | Region | the Matched Region | Expect Value |
| 7tm_1 | 41 . . . 290 | 46/276 (17%) | 5.5e−25 |
| | | 167/276 (61%) | |

### Example 24

The NOV24 clone was analyzed, and the nucleotide and encoded polypeptide sequences are shown in Table 24A.

TABLE 24A
NOV24 Sequence Analysis

| | | |
|---|---|---|
| NOV24a, CG53482-01 | SEQ ID NO: 311 | 1008 bp |
| DNA Sequence | ORF Start: ATG at 27 | ORF Stop: TAG at 999 |

AGCTGGAGATCTGGAACTTCCACAGCATGGAGCTCTGGAACTACCACAGCATGGAGCTCTGGAACTTC
ACCTTGGGAAGTGGCTTCATTTTGGTGGGGATTCTGAATGACAGTGGGTCTCCTGAACTGCTCTGTGC
TACAATTACAATCCTATACTTGTTGGCCCTGATCAGCAATGGCCTACTGCTCCTGGCTATCACCATGG
AAGCCCGGCTCCACATGCCCATGTACCTCCTGCTTGGGCAGCTCTCTCTCATGGACCTCCTGTTCACA
TCTGTTGTCACTCCCAAGGCCCTTGCGGACTTTCTGCGCAGAGAAAACACCATCTCCTTTGGAGGCTG
TGCCCTTCAGATGTTCCTGGCACTGACAATGGGTGGTGCTGAGGACCTCCTACTGGCCTTCATGGCCT
ATGACAGGTATGTGGCCATTTGTCATCCTCTGACATACATGACCCTCATGAGCTCAAGAGCCTGCTGG
CTCATGGTGGCCACGTCCTGGATCCTGGCATCCCTAAGTGCCCTAATATATACCGTGTATACCATGCA
CTATCCCTTCTGCAGGGCCCAGGAGATCAGGCATCTTCTCTGTGAGATCCCACACTTGCTGAAGTTGG
CCTGTGCTGATACCTCCAGATATGAGCTCATGGTATATGTGATGGGTGTGACCTTCCTGATTCCCTCT
CTTGCTGCTATACTGGCCTCCTATACACAAATTCTACTCACTGTGCTCCATATGCCATCAAATGAGGG
GAGGAAGAAAGCCCTTGTCACCTGCTCTTCCCACCTGACTGTGGTTGGGATGTTCTATGGAGCTGCCA
CATTCATGTATGTCTTGCCCAGTTCCTTCCACAGCACCAGACAAGACAACATCATCTCTGTTTTCTAC
ACAATTGTCACTCCAGCCCTGAATCCACTCATCTACAGCCTGAGGAATAAGGAGGTCATGCGGGCCTT
GAGGAGGGTCCTGGGAAAATACATGCTGCCAGCACACTCCACGCTCTAGGGAAGGA

| | | | |
|---|---|---|---|
| NOV24a, CG53482-01 | SEQ ID NO: 312 | 324 aa | MW at 36344.8kD |
| Protein Sequence | | | |

MELWNYHSMELWNFTLGSGFILVGILNDSGSPELLCATITILYLLALISNGLLLLAITMEARLHMPMY
LLLGQLSLMDLLFTSVVTPKALADFLRRENTISFGGCALQMFLALTMGGAEDLLLAFMAYDRYVAICH
PLTYMTLMSSRACWLMVATSWILASLSALIYTVYTMHYPFCRAQEIRHLLCEIPHLLKLACADTSRYE
LMVYVMGVTFLIPSLAAILASYTQILLTVLHMPSNEGRKKALVTCSSHLTVVGMFYGAATFMYVLPSS
FHSTRQDNIISVFYTIVTPALNPLIYSLRNKEVMRALRRVLGKYMLPAHSTL

| | | |
|---|---|---|
| NOV24b, CG53482-02 | SEQ ID NO: 313 | 1050 bp |
| DNA Sequence | ORF Start: ATG at 72 | ORF Stop: TAG at 1020 |

AAACTAGAGTTCATCTTAGCAPAAATTCATGAAGTATCCATCTTGTTCTAGGTGATGAAAGAAACCAC
AGCATGGAGCTCTGGAACTTCACCTTGGGAAGTGGCTTCATTTTGGTGGGGATTCTGAATGACAGTGG
GTCTCCTGAACTGCTCTGTGCTACAATTACAATCCTATACTTGTTGGCCCTGATCAGCAATGGCCTAC
TGCTCCTGGCTATCACCATGGAAGCCCGGCTCCACATGCCCATGTACCTCCTGCTTGGGCAGCTCTCT
CTCATGGACCTCCTGTTCACATCTGTTGTCACTCCCAAGGCCCTTGCGGACTTTCTGCGCAGAGAAAA
CACCATCTCCTTTGGAGGCTGTGCCCTTCAGATGTTCCTGGCACTGACAATGGGTGGTGCTGAGGACC
TCCTACTGGCCTTCATGGCCTATGACAGGTATGTGGCCATTTGTCATCCTCTGACATACATGACCCTC
ATGAGCTCAAGAGCCTGCTGGCTCATGGTGGCCACGTCCTGGATCCTGGCATCCCTAAGTGCCCTAAT
ATATACCGTGTATACCATGCACTATCCCTTCTGCAGGGCCCAGGAGATCAGGCATCTTCTCTGTGAGA
TCCCACACTTGCTGAAGGTGGCCTGTGCTGATACCTCCAGATATGAGCTCATGGTATATGTGATGGGT
GTGACCTTCCTGATTCCCTCTCTTGCTGCTATACTGGCCTCCTATACACAAATTCTACTCACTGTGCT
CCATATGCCATCAAATGAGGGGAGGAAGAAAGCCCTTGTCACCTGCTCTTCCCACCTGACTGTGGTTG
GGATGTTCTATGGAGCTGCCACATTCATGTATGTCTTGCCCAGTTCCTTCCACAGCACCAGACAAGAC
AACATCATCTCTGTTTTCTACACAATTGTCACTCCAGCCCTGAATCCACTCATCTACAGCCTGAGGAA
TAAGGAGGTCATGCGGGCCTTGAGGAGGGTCCTGGGAAAATACATGCTGCCAGCACACTCCACGCTCT
AGGGAAGGATCATGGCTAGCTTCCAGAATT

| | | | |
|---|---|---|---|
| NOV24b, CG53482-02 | SEQ ID NO: 314 | 316 aa | MW at 35269.6kD |
| Protein Sequence | | | |

MELWNFTLGSGFILVGILNDSGSPELLCATITILYLLALISNGLLLLAITMEARLHMPMYLLLGQLSL
MDLLFTSVVTPKALADFLRRENTISFGGCALQMFLALTMGGAEDLLLAFMAYDRYVAICHPLTYMTLM
SSRACWLMVATSWILASLSALIYTVYTNHYPFCRAQEIRHLLCEIPHLLKVACADTSRYELMVYVMGV
TFLIPSLAAILASYTQILLTVLHMPSNEGRKKALVTCSSHLTVVGMFYGAATFMYVLPSSFHSTRQDN
IISVFYTIVTPALNPLIYSLRNKEVMRALRRVLGKYMLPAHSTL

| | | |
|---|---|---|
| NOV24c, CG53482-03 | SEQ ID NO: 315 | 1010 bp |
| DNA Sequence | ORF Start: at 4 | ORF Stop: TAG at 1000 |

TAGCTGGAGATCTGGAACTTCCACAGCATGGAGCTCTGGAACTACCACAGCATGGAGCTCTGGAACTT
CACCTTGGGAAGTGGCTTCATTTTGGTGGGGATTCTGAATGACAGTGGGTCTCCTGAACTGCTCTGTG
CTACAATTACAATCCTATACTTGTTGGCCCTGATCAGCAATGGCCTACTGCTCCTGGCTATCACCATG
GAAGCCCGGCTCCACATGCCCATGTACCTCCTGCTTGGGCAGCTCTCTCTCATGGACCTCCTGTTCAC
ATCTGTTGTCACTCCCAAGGCCCTTGCGGACTTTCTGCGCAGAGAAAACACCATCTCCTTTGGAGGCT
GTGCCCTTCAGATGTTCCTGGCACTGACAATGGGTGGTGCTGAGGACCTCCTACTGGCCTTCATGGCC
TATGACAGGTATGTGGCCATTTGTCATCCTCTGACATACATGACCCTCATGAGCTCAAGAGCCTGCTG
GCTCATGGTGGCCACGTCCTGGATCCTGGCATCCCTAAGTGCCCTAATATATACCGTGTATACCATGC
ACTATCCCTTCTGCAGGGCCCAGGAGATCAGGCATCTTCTCTGTGAGATCCCACACTTGCTGAAGTTG
GCCTGTGCTGATACCTCCAGATATGAGCTCATGGTATATGTGATGGGTGTGACCTTCCTGATTCCCTC
TCTTGCTGCTATACTGGCCTCCTATACACAAATTCTACTCACTGTGCTCCATATGCCATCAAATGAGG
GGAGGAAGAAAGCCCTTGTCACCTGCTCTTCCCACCTGACTGTGGTTGGGATGTTCTATGGAGCTGCC
ACATTCATGTATGTCTTGCCCAGTTCCTTCCACAGCACCAGACAAGACAACATCATCTCTGTTTTCTA
CACAATTGTCACTCCAGCCCTGAATCCACTCATCTACAGCCTGAGGAATAAGGAGGTCATGCGGGCCT
TGAGGAGGGTCCTGGGAAAATACATGCTGCCAGCACACTCCACGCTCTAGGGAAGGAA

| | | | |
|---|---|---|---|
| NOV24c, CG53482-03 | SEQ ID NO: 316 | 332 aa | MW at 37371.9kD |
| Protein Sequence | | | |

LEIWNFHSMELNNYHSMELWNFTLGSGFILVGILNDSGSPELLCATITILYLLALISNGLLLLAITME
ARLHMPMYLLLGQLSLMDLLFTSVVTPKALADFLRRENTISFGGCALQMFLALTMGGABDLLLAFMAY
DRYVAICHPLTYMTLMSSRACWLMVATSWILASLSALIYTVYTMHYPFCRAQEIRHLLCEIPHLLKLA
CADTSRYELMVYVMGVTFLIPSLAAILASYTQILLTVLHMPSNEGRKKALVTCSSHLTVVGMFYGAAT
FMYVLPSSFHSTRQDNIISVFYTIVTPALNPLIYSLRNKEVMRALRRVLGKYMLPAHSTL

| | | |
|---|---|---|
| NOV24d, SNP13373787 of | SEQ ID NO: 317 | 1008 bp |
| CG53482-01, DNA Sequence | ORF Start: ATG at 27 | ORF Stop: TAG at 999 |
| | SNP Pos: 430 | SNP Change: G to A |

AGCTGGAGATCTGGAACTTCCACAGCATGGAGCTCTGGAACTACCACAGCATGGAGCTCTGGAACTTC
ACCTTGGGAAGTGGCTTCATTTTGGTGGGGATTCTGAATGACAGTGGGTCTCCTGAACTGCTCTGTGC
TACAATTACAATCCTATACTTGTTGGCCCTGATCAGCAATGGCCTACTGCTCCTGGCTATCACCATGG
AAGCCCGGCTCCACATGCCCATGTACCTCCTGCTTGGGCAGCTCTCTCTCATGGACCTCCTGTTCACA
TCTGTTGTCACTCCCAAGGCCCTTGCGGACTTTCTGCGCAGAGAAAACACCATCTCCTTTGGAGGCTG
TGCCCTTCAGATGTTCCTGGCACTGACAATGGGTGGTGCTGAGGACCTCCTACTGGCCTTCATGGCCT
ATGACAGGTATGTGGCCATTTATCATCCTCTGACATACATGACCCTCATGAGCTCAAGAGCCTGCTGG
CTCATGGTGGCCACGTCCTGGATCCTGGCATCCCTAAGTGCCCTAATATATACCGTGTATACCATGCA
CTATCCCTTCTGCAGGGCCCAGGAGATCAGGCATCTTCTCTGTGAGATCCCACACTTGCTGAAGTTGG
CCTGTGCTGATACCTCCAGATATGAGCTCATGGTATATGTGATGGGTGTGACCTTCCTGATTCCCTCT
CTTGCTGCTATACTGGCCTCCTATACACAAATTCTACTCACTGTGCTCCATATGCCATCAAATGAGGG
GAGGAAGAAAGCCCTTGTCACCTGCTCTTCCCACCTGACTGTGGTTGGGATGTTCTATGGAGCTGCCA
CATTCATGTATGTCTTGCCCAGTTCCTTCCACAGCACCAGACAAGACAACATCATCTCTGTTTTCTAC
ACAATTGTCACTCCAGCCCTGAATCCACTCATCTACAGCCTGAGGAATAAGGAGGTCATGCGGGCCTT
GAGGAGGGTCCTGGGAAAATACATGCTGCCAGCACACTCCACGCTCTAGGGAAGGA

| | | | |
|---|---|---|---|
| NOV24d, SNP13373787 of | SEQ ID NO: 318 | 324 aa | MW at 36404.8kD |
| CG53482-01, Protein | SNP Pos: 135 | | SNP Change: Cys to Tyr |
| Sequence | | | |

MELWNYHSMELWNFTLGSGFILVGILNDSGSPELLCATITILYLLALISNGLLLLAITMEARLHMPMY
LLLGQLSLMDLLFTSVVTPKALADFLRRENTISFGGCALQMFLALTMGGAEDLLLAFMAYDRYVAIYH
PLTYMTLMSSRACWLMVATSWILASLSALIYTVYTMHYPFCRAQEIRHLLCEIPHLLKLACADTSRYE
LMVYVMGVTFLIPSLAAILASYTQILLTVLHMPSNEGRKKALVTCSSHLTVVGMFYGAATFMYVLPSS
FHSTRQDNIISVFYTIVTPALNPLIYSLRNKEVMRALRRVLGKYMLPAHSTL

| | | |
|---|---|---|
| NOV24e, SNP13373786 of | SEQ ID NO: 319 | 1008 bp |
| CG53482-01, DNA Sequence | ORF Start: ATG at 27 | ORF Stop: TAG at 999 |
| | SNP Pos: 442 | SNP Change: C to A |

AGCTGGAGATCTGGAACTTCCACAGCATGGAGCTCTGGAACTACCACAGCATGGAGCTCTGGAACTTC
ACCTTGGGAAGTGGCTTCATTTTGGTGGGGATTCTGAATGACAGTGGGTCTCCTGAACTGCTCTGTGC
TACAATTACAATCCTATACTTGTTGGCCCTGATCAGCAATGGCCTACTGCTCCTGGCTATCACCATGG
AAGCCCGGCTCCACATGCCCATGTACCTCCTGCTTGGGCAGCTCTCTCTCATGGACCTCCTGTTCACA
TCTGTTGTCACTCCCAAGGCCCTTGCGGACTTTCTGCGCAGAGAAAACACCATCTCCTTTGGAGGCTG
TGCCCTTCAGATGTTCCTGGCACTGACAATGGGTGGTGCTGAGGACCTCCTACTGGCCTTCATGGCCT
ATGACAGGTATGTGGCCATTTGTCATCCTCTGAAATACATGACCCTCATGAGCTCAAGAGCCTGCTGG
CTCATGGTGGCCACGTCCTGGATCCTGGCATCCCTAAGTGCCCTAATATATACCGTGTATACCATGCA
CTATCCCTTCTGCAGGGCCCAGGAGATCAGGCATCTTCTCTGTGAGATCCCACACTTGCTGAAGTTGG
CCTGTGCTGATACCTCCAGATATGAGCTCATGGTATATGTGATGGGTGTGACCTTCCTGATTCCCTCT
CTTGCTGCTATACTGGCCTCCTATACACAAATTCTACTCACTGTGCTCCATATGCCATCAAATGAGGG
GAGGAAGAAAGCCCTTGTCACCTGCTCTTCCCACCTGACTGTGGTTGGGATGTTCTATGGAGCTGCCA
CATTCATGTATGTCTTGCCCAGTTCCTTCCACAGCACCAGACAAGACAACATCATCTCTGTTTTCTAC
ACAATTGTCACTCCAGCCCTGAATCCACTCATCTACAGCCTGAGGAATAAGGAGGTCATGCGGGCCTT
GAGGAGGGTCCTGGGAAATACATGCTGCCAGCACACTCCACGCTCTAGGGAAGGA

| | | | |
|---|---|---|---|
| NOV24e, SNP13373786 of | SEQ ID NO: 320 | 324 aa | MW at 36371.9kD |
| CG53482-01, Protein | SNP Pos: 139 | | SNP Change: Thr to Lys |
| Sequence | | | |

MELWNYHSMELWNFTLGSGFILVGILNDSGSPELLCATITILYLLALISNGLLLLAITMEARLHMPMY
LLLGQLSLMDLLFTSVVTPKALADFLRRENTISFGGCALQMFLALTMGGAEDLLLAFMAYDRYVAICH
PLKYMTLMSSRACWLMVATSWILASLSALIYTVYTMHYPFCRAQEIRHLLCEIPHLLKLACADTSRYE
LMVYVMGVTFLIPSLAAILASYTQILLTVLHMPSNEGRKKALVTCSSHLTVVGMFYGAATFMYVLPSS
FHSTRQDNIISVFYTIVTPALNPLIYSLRNKEVMRALRRVLGKYMLPAHSTL

| | | |
|---|---|---|
| NOV24f, SNP13373785 of | SEQ ID NO: 321 | 1008 bp |
| CG53482-01, DNA Sequence | ORF Start: ATG at 27 | ORF Stop: TAG at 999 |
| | SNP Pos: 754 | SNP Change: A to G |

AGCTGGAGATCTGGAACTTCCACAGCATGGAGCTCTGGAACTACCACAGCATGGAGCTCTGGAACTTC
ACCTTGGGAAGTGGCTTCATTTTGGTGGGGATTCTGAATGACAGTGGGTCTCCTGAACTGCTCTGTGC
TACAATTACAATCCTATACTTGTTGGCCCTGATCAGCAATGGCCTACTGCTCCTGGCTATCACCATGG
AAGCCCGGCTCCACATGCCCATGTACCTCCTGCTTGGGCAGCTCTCTCTCATGGACCTCCTGTTCACA
TCTGTTGTCACTCCCAAGGCCCTTGCGGACTTTCTGCGCAGAGAAAACACCATCTCCTTTGGAGGCTG
TGCCCTTCAGATGTTCCTGGCACTGACAATGGGTGGTGCTGAGGACCTCCTACTGGCCTTCATGGCCT
ATGACAGGTATGTGGCCATTTGTCATCCTCTGACATACATGACCCTCATGAGCTCAAGAGCCTGCTGG
CTCATGGTGGCCACGTCCTGGATCCTGGCATCCCTAAGTGCCCTAATATATACCGTGTATACCATGCA
CTATCCCTTCTGCAGGGCCCAGGAGATCAGGCATCTTCTCTGTGAGATCCCACACTTGCTGAAGTTGG
CCTGTGCTGATACCTCCAGATATGAGCTCATGGTATATGTGATGGGTGTGACCTTCCTGATTCCCTCT
CTTGCTGCTATACTGGCCTCCTATACACAAATTCTACTCACTGTGCTCCATATGCCATCAAATGAGGG
GAGGAGGAAAGCCCTTGTCACCTGCTCTTCCCACCTGACTGTGGTTGGGATGTTCTATGGAGCTGCCA
CATTCATGTATGTCTTGCCCAGTTCCTTCCACAGCACCAGACAAGACAACATCATCTCTGTTTTCTAC
ACAATTGTCACTCCAGCCCTGAATCCACTCATCTACAGCCTGAGGAATAAGGAGGTCATGCGGGCCTT
GAGGAGGGTCCTGGGAAATACATGCTGCCAGCACACTCCACGCTCTAGGGAAAGGA

| | | | |
|---|---|---|---|
| NOV24f, SNP13373785 of | SEQ ID NO: 322 | 324 aa | MW at 36372.8kD |
| CG53482-01, Protein | SNP Pos: 243 | | SNP Change: Lys to Arg |
| Sequence | | | |

MELWNYHSMELWNFTLGSGFILVGILNDSGSPELLCATITILYLLALISNGLLLLAITMEARLHMPMY
LLLGQLSLMDLLFTSVVTPKALADFLRRENTISFGGCALQMFLALTMGGAEDLLLAFMAYDRYVAICH
PLKYMTLMSSRACWLMVATSWILASLSALIYTVYTMHYPFCRAQEIRHLLCEIPHLLKLACADTSRYE
LMVYVMGVTFLIPSLAAILASYTQILLTVLHMPSNEGRRKALVTCSSHLTVVGMFYGAATFMYVLPSS
FHSTRQDNIISVFYTIVTPALNPLIYSLRNKEVMRALRRVLGKYMLPAHSTL

A ClustalW comparison of the above protein sequences yields the following sequence alignment shown in Table 24B.

TABLE 24B
Comparison of the NOV24 protein sequences.

NOV24a --------MELWNYHSMELWNFTLGSGFILVGILNDSGSPELLCATITILYLLALISNGL
NOV24b ----------------MELWNFTLGSGFILVGILNDSGSPELLCATITILYLLALISNGL
NOV24c LEIWNFHSMELWNYHSMELWNFTLGSGFILVGILNDSGSPELLCATITILYLLALISNGL
NOV24a LLLAITMEARLHMPMYLLLGQLSLMDLLFTSVVTPKALADFLRRENTISFGGCALQMFLA
NOV24b LLLAITMEARLHMPMYLLLGQLSLMDLLFTSVVTPKALADFLRRENTISFGGCALQMFLA
NOV24c LLLAITMEARLHMPMYLLLGQLSLMDLLFTSVVTPKALADFLRRENTISFGGCALQMFLA
NOV24a LTMGGAEDLLLAFMAYDRYVAICHPLTYMTLMSSRACWLMVATSWILASLSALIYTVYTM
NOV24b LTMGGAEDLLLAFMAYDRYVAICHPLTYMTLMSSRACWLMVATSWILASLSALIYTVYTM
NOV24c LTMGGAEDLLLAFMAYDRYVAICHPLTYMTLMSSRACWLMVATSWILASLSALIYTVYTM
NOV24a HYPFCRAQEIRHLLCEIPHLLKLACADTSRYELMVYVMGVTFLIPSLAAILASYTQILLT
NOV24b HYPFCRAQEIRHLLCEIPHLLKVACADTSRYELMVYVMGVTFLIPSLAAILASYTQILLT
NOV24c HYPFCRAQEIRHLLCEIPHLLKLACADTSRYELMVTVMGVTFLIPSLAAILASYTQILLT
NOV24a VLHMPSNEGRKKALVTCSSHLTVVGMFYGAATFMYVLPSSFHSTRQDNIISVFYTIVTPA
NOV24b VLHMPSNEGRKKALVTCSSHLTVVGMFYGAATFMYVLPSSFHSTRQDNIISVFYTIVTPA
NOV24c VLHMPSNEGRKKALVTCSSHLTVVGMFYGAATFMYVLPSSFHSTRQDNIISVFYTIVTPA
NOV24a LNPLIYSLRNKEVMRALRRVLGKYMLPAHSTL
NOV24b LNPLIYSLRNKEVMRALRRVLGKYMLPAHSTL
NOV24c LNPLIYSLRNKEVMRALRRVLGKYMLPAHSTL
NOV24a (SEQ ID NO: 312)
NOV24b (SEQ ID NO: 314)
NOV24c (SEQ ID NO: 316)

Further analysis of the NOV24a protein yielded the following properties shown in Table 24C.

TABLE 24C
Protein Sequence Properties NOV24a

| | |
|---|---|
| SignalP analysis: | Cleavage site between residues 62 and 63 |
| PSORT II analysis: | |

| | |
|---|---|
| PSG: | a new signal peptide prediction method |
| | N-region: length 10; pos.chg 0; neg.chg 2 |
| | H-region: length 17; peak value 0.00 |
| | PSG score: −4.40 |
| GvH: | von Heijne's method for signal seq. recognition |
| | GvH score (threshold: −2.1): −3.29 |
| | possible cleavage site: between 56 and 57 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 6 | | |
| | INTEGRAL | Likelihood = −7.75 | Transmembrane 41-57 |
| | INTEGRAL | Likelihood = −1.65 | Transmembrane 69-85 |
| | INTEGRAL | Likelihood = −2.07 | Transmembrane 150-166 |
| | INTEGRAL | Likelihood = −5.10 | Transmembrane 207-223 |
| | INTEGRAL | Likelihood = −0.80 | Transmembrane 253-269 |
| | INTEGRAL | Likelihood = −0.80 | Transmembrane 281-297 |

| | |
|---|---|
| PERIPHERAL | Likelihood = 0.85 (at 109) |

| | |
|---|---|
| | ALOM score: −7.75 (number of TMSs: 6) |
| MTOP: | Prediction of membrane topology (Hartmann et al.) |
| | Center position for calculation: 48 |
| | Charge difference: 2.5 C(0.5) − N(−2.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 4.76 |
| Hyd Moment(95): | 6.14 | G content: | 0 |
| D/E content: | 2 | S/T content: | 1 |

Score: −6.93

Gavel: prediction of cleavage sites for mitochondrial preseq

cleavage site motif not found

NUCDISC: discrimination of nuclear localization signals

pat4: none
pat7: none
bipartite: none
content of basic residues: 6.2%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG53482-01 is end (k = 9)

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU97928 | Novel odourant receptor NOV7 | 1 . . . 324 | 324/324 (100%) | 0.0 |
| | protein - Unidentified, 324 aa. | 1 . . . 324 | 324/324 (100%) | |
| | [WO200236632-A2, 10-MAY- | | | |
| | 2002] | | | |
| AAU97927 | Novel odourant receptor NOV6 | 1 . . . 324 | 324/324 (100%) | 0.0 |
| | protein - Unidentified, 324 aa. | 1 . . . 324 | 324/324 (100%) | |
| | [WO200236632-A2, 10-MAY- | | | |
| | 2002] | | | |
| AAU07088 | Human odorant receptor (OR) | 1 . . . 324 | 324/324 (100%) | 0.0 |
| | polypeptide #5 - Homo sapiens, | 1 . . . 324 | 324/324 (100%) | |
| | 324 aa. [WO200157215-A2, 09- | | | |
| | AUG-2001] | | | |
| AAU85234 | G-coupled olfactory receptor #95 - | 9 . . . 324 | 315/316 (99%) | e−180 |
| | Homo sapiens, 316 aa. | 1 . . . 316 | 316/316 (99%) | |
| | [WO200198526-A2, 27-DEC- | | | |
| | 2001] | | | |
| AAU95574 | Human olfactory and pheromone | 9 . . . 324 | 315/316 (99%) | e−180 |
| | G protein-coupled receptor #61 - | 1 . . . 316 | 316/316 (99%) | |
| | Homo sapiens, 316 aa. | | | |
| | [WO200224726-A2, 28-MAR- | | | |
| | 2002] | | | |

In a BLAST search of public sequence databases, the NOV24a protein was found to have homology to the proteins shown in the BLASTP data in Table 24E.

TABLE 24E
Public BLASTP Results for NOV24a

| | | NOV24a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD57894 | Sequence 11 from Patent | 1 . . . 324 | 324/324 (100%) | 0.0 |
| | WO0236632 - Homo sapiens | 1 . . . 324 | 324/324 (100%) | |
| | (Human), 324 aa. | | | |
| Q9H205 | Olfactory receptor 2AG1 | 9 . . . 324 | 315/316 (99%) | e−179 |
| | (HT3) - Homo sapiens | 1 . . . 316 | 316/316 (99%) | |
| | (Human), 316 aa. | | | |
| Q8VGU0 | Olfactory receptor MOR283-2 - | 9 . . . 324 | 264/316 (83%) | e−149 |
| | Mus musculus (Mouse), 316 | 1 . . . 316 | 284/316 (89%) | |
| | aa. | | | |
| Q9EPF7 | T2 olfactory receptor - Mus | 9 . . . 324 | 262/316 (82%) | e−149 |
| | musculus (Mouse), 316 aa. | 1 . . . 316 | 284/316 (88%) | |
| Q8VFM5 | Olfactory receptor MOR283-4 - | 9 . . . 323 | 253/315 (80%) | e−145 |
| | Mus musculus (Mouse), 316 | 1 . . . 315 | 280/315 (88%) | |
| | aa. | | | |

PFam analysis indicates that the NOV24a protein contains the domains shown in the Table 24F.

TABLE 24F
Domain Analysis of NOV24a

| | | Identities/ | |
|---|---|---|---|
| | NOV24a Match | Similarities for | |
| Pfam Domain | Region | the Matched Region | Expect Value |
| 7tm_1 | 49 . . . 298 | 62/277 (22%) | 1.3e−33 |
| | | 173/277 (62%) | |

### Example 25

The NOV25 clone was analyzed, and the nucleotide and encoded polypeptide sequences are shown in Table 25A.

TABLE 25A
NOV25 Sequence Analysis

| | | |
|---|---|---|
| NOV25a, CG53530-03 | SEQ ID NO: 323 | 1016 bp |
| DNA Sequence | ORF Start: ATG at 7 | ORF Stop: TAG at 961 |

TACCAGATGAATCCAGCAAATCATTCCCAGGTGGCAGGATTTGTTCTACTGGGGCTCTCTCAGGTTTG
GGAGCTTCGGTTTGTTTTCTTCACTGTTTTCTCTGCTGTGTATTTTATGACTGTAGTGGGAAACCTTC
TTATTGTGGTCATAGTGACCTCCGACCCACACCTGCACACAACCATGTATTTTCTCTTGGGCAATCTT
TCTTTCCTGGACTTTTGCTACTCTTCCATCACAGCACCTAGGATGCTGGTTGACTTGCTCTCAGGCAA
CCCTACCATTTCCTTTGGTGGATGCCTGACTCAACTCTTCTTCTTCCACTTCATTGGAGGCATCAAGA
TCTTCCTGCTGACTGTCATGGCGTATGACCGCTACATTGCCATTTCCCAGCCCCTGCACTACACGCTC
ATTATGAATCAGACTGTCTGTGCACTCCTTATGGCAGCCTCCTGGGTGGGGGGCTTCATCCACTCCAT
AGTACAGATTGCATTGACTATCCAGCTGCCATTCTGTGGGCCTGACAAGCTGGACAACTTTTATTGTG
ATGTGCCTCAGCTGATCAAATTGGCCTGCACAGATACCTTTGTCTTAGAGCTTTTAATGGTGTCTAAC
AATGGCCTGGTGACCCTGATGTGTTTTCTGGTGCTTCTGGGATCGTACACAGCACTGCTAGTCATGCT
CCGAAGCCACTCACGGGAGGGCCGCAGCAAGGCCCTGTCTACCTGTGCCTCTCACATTGCTGTGGTGA
CCTTAATCTTTGTGCCTTGCATCTACGTCTATACAAGGCCTTTTCGGACATTCCCCATGGACAAGGCC
GTCTCTGTGCTATACACAATTGTCACCCCCATGCTGAATCCTGCCATCTATACCCTGAGAAACAAGGA
AGTGATCATGGCCATGAAGAAGCTGTGGAGGAGGAAAAAGGACCCTATTGGTCCCCTGGAGCACAGAC
CCTTACATTAGCAGAGGCAGTGACCTGAGAATCTGAAAGATGCTACAGGGTATTAGCAGAGGCA

| | | | |
|---|---|---|---|
| NOV25a, CG53530-03 | SEQ ID NO: 324 | 318 aa | MW at 35770.3kD |
| Protein Sequence | | | |

MNPANHSQVAGFVLLGLSQVWELRFVFFTVFSAVYFMTVVGNLLIVVIVTSDPHLHTTMYFLLGNLSF
LDFCYSSITAPRMLVDLLSGNPTISFGGCLTQLFFFHFIGGIKIFLLTVMAYDRYIAISQPLHYTLIM
NQTVCALLMAASWVGGFIHSIVQIALTIQLPFCGPDKLDNFYCDVPQLIKLACTDTFVLELLMVSNNG
LVTLMCFLVLLGSYTALLVMLRSHSREGRSKALSTCASHIAVVTLIFVPCIYVYTRPFRTFPMDKAVS
VLYTIVTPHLNPAIYTLRNKEVIMANKKLWRRKKDPIGPLEHRPLH

| | | |
|---|---|---|
| NOV25b, CG53530-01 | SEQ ID NO: 325 | 1077 bp |
| DNA Sequence | ORF Start: ATG at 31 | ORF Stop: TAG at 985 |

CAGGTTCATTGACAAGGTCATACCAACCAGATGAATCCAGCAAATCATTCCCAGGTGGCAGGATTTGT
TCTACTGGGGCTCTCTCAGGTTTGGGAGCTTCGGTTTGTTTTCTTCACTGTTTTCTCTGCTGTGTATT
TTATGACTGTAGTGGGAAACCTTCTTATTGTGGTCATAGTGACCTCCGACCCACACCTGCACACAACC
ATGTATTTTCTCTTGGGCAATCTTTCTTTCCTGGACTTTTGCTACTCTTCCATCACAGCACCTAGGAT
GCTGGTTGACTTGCTCTCAGGCAACCCTACCATTTCCTTTGGTGGATGCCTGACTCAACTCTTCTTCT
TCCACTTCATTGGAGGCATCAAGATCTTCCTGCTGACTGTCATGGCGTATGACCGCTACATTGCCATT
TCCCAGCCCCTGCACTACACGCTCATTATGAATCAGACTGTCTGTGCACTCCTTATGGCAGCCTCCTG
GGTGGGGGGCTTCATCCACTCCATAGTACAGATTGCATTGACTATCCAGCTGCCATTCTGTGGGCCTG
ACAAGCTGGACAACTTTTATTGTGATGTGCCTCAGCTGATCAAATTGGCCTGCACAGATACCTTTGTC
TTAGAGCTTTTAATGGTGTCTAACAATGGCCTGGTGACCCTGATGTGTTTTCTGGTGCTTCTGGGATC
GTACACAGCACTGCTAGTCATGCTCCGAAGCCACTCACGGGAGGGCCGCAGCAAGGCCCTGTCTACCT
GTGCCTCTCACATTGCTGTGGTGACCTTAATCTTTGTGCCTTGCATCTACGTCTATACAAGGCCTTTT
CGGACATTCCCCATGGACAAGGCCGTCTCTGTGCTATACACAATTGTCACCCCCATGCTGAATCCTGC
CATCTATACCCTGAGAAACAAGGAAGTGATCATGGCCATGAAGAAGCTGTGGAGGAGGAAAAAGGACC
CTATTGGTCCCCTGGAGCACAGACCCTTACATTAGCAGAGGCAGTGACCTGAGAATCTGAAAGATGCT
ACAGGGTATTAGCAGAGGCAGTGACCTGAGAATCTGAAAGATGCTACAGGGTATTAG

| | | | |
|---|---|---|---|
| NOV25b, CG53530-01 | SEQ ID NO: 326 | 318 aa | MW at 35770.3kD |
| Protein Sequence | | | |

MNPANHSQVAGFVLLGLSQVWELRFVFFTVFSAVYFMTVVGNLLIVVIVTSDPHLHTTMYFLLGNLSF
LDFCYSSITAPRMLVDLLSGNPTISFGGCLTQLFFFHFIGGIKIFLLTVMAYDRYIAISQPLHYTLIM
NQTVCALLMAASWVGGFIHSIVQIALTIQLPFCGPDKLDMFYCDVPQLIKLACTDTFVLELLMVSNNG
LVTLMCFLVLLGSYTALLVMLRSHSREGRSKALSTCASHIAVVTLIFVPCIYVYTRPFRTFPMDKAVS
VLYTIVTPMLNPAIYTLRNIEVIMAHKKLWRRKKDPIGPLEHRPLH

| | | |
|---|---|---|
| NOV25c,CG53530-02 | SEQ ID NO: 327 | 1077 bp |
| DNA Sequence | ORF Start: ATG at 31 | ORF Stop: TAG at 985 |

CAGGTTCATTGACAAGGTCATACCAACCAGATGAATCCAGCAAATCATTCCCAGGTGGCAGGATTTGT
TCTACTGGGGCTCTCTCAGGTTTGGGAGCTTCGGTTTGTTTTCTTCACTGTTTTCTCTGCTGTGTATT
TTATGACTGTAGTGGGAAACCTTCTTATTGTGGTCATAGTGACCTCCGACCCACACCTGCACACAACC
ATGTATTTTCTCTTGGGCAATCTTTCTTTCCTGGACTTTTGCTACTCTTCCATCACAGCACCTAGGAT
GCTGGTTGACTTGCTCTCAGGCAACCCTACCATTTCCTTTGGTGGATGCCTGACTCAACTCTTCTTCT
TCCACTTCATTGGAGGCATCAAGATCTTCCTGCTGACTGTCATGGCGTATGACCGCTACATTGCCATT
TCCCAGCCCCTGCACTACACGCTCATTATGAATCAGACTGTCTGTGCACTCCTTATGGCAGCCTCCTG
GGTGGGGGGCTTCATCCACTCCATAGTACAGATTGCATTGACTATCCAGCTGCCATTCTGTGGGCCTG
ACAAGCTGGACAACTTTTATTGTGATGTGCCTCAGCTGATCAAATTGGCCTGCACAGATACCTTTGTC
TTAGAGCTTTTAATGGTGTCTAACAATGGCCTGGTGACCCTGATGTGTTTTCTGGTGCTTCTGGGATC
GTACACAGCACTGCTAGTCATGCTCCGAAGCCACTCACGGGAGGGCCGCAGCAAGGCCCTGTCTACCT
GTGCCTCTCACATTGCTGTGGTGACCTTAATCTTTGTGCCTTGCATCTACGTCTATACAAGGCCTTTT
CGGACATTCCCCATGGACAAGGCCGTCTCTGTGCTATACACAATTGTCACCCCCATGCTGAATCCTGC
CATCTATACCCTGAGAAACAAGGAAGTGATCATGGCCATGAAGAAGCTGTGGAGGAGGAAAAAGGACC
CTATTGGTCCCCTGGAGCACAGACCCTTACATTAGCAGAGGCAGTGACCTGAGAATCTGAAAGATGCT
ACAGGGTATTAGCAGAGGCAGTGACCTGAGAATCTGAAAGATGCTACAGGGTATTAG

| | | | |
|---|---|---|---|
| NOV25c, CG53530-02 | SEQ ID NO: 328 | 318 aa | MW at 35770.3kD |
| Protein Sequence | | | |

MNPANHSQVAGFVLLGLSQVWELRFVFFTVFSAVYFMTVVGNLLIVVIVTSDPHLHTTMYFLLGNLSF
LDFCYSSITAPRNLVDLLSGNPTISFGGCLTQLFFFHFIGGIKIFLLTVMAYDRYIAISQPLHYTLIM
NQTVCALLMAASWVGGFIHSIVQIALTIQLPFCGPDKLDNFYCDVPQLIKLACTDTFVLELLMVSNNG
LVTLMCFLVLLGSYTALLVMLRSHSREGRSKALSTCASHIAVVTLIFVPCIYVYTRPFRTFPMDKAVS
VLYTIVTPMLNPAIYTLRNKEVIMAMKKLWRRKKDPIGPLEHRPLH

A ClustalW comparison of the above protein sequences yields the following sequence alignment shown in Table 25B.

TABLE 25B
Comparison of the NOV25 protein sequences.

NOV25a MNPANHSQVAGFVLLGLSQVWELRFVFFTVFSAVYFMTVVGNLLIVVIVTSDPHLHTTMY
NOV25b MNPANHSQVAGFVLLGLSQVWELRFVFFTVFSAVYFMTVVGNLLIVVIVTSDPHLHTTMY
NOV25c MNPANHSQVAGFVLLGLSQVWELRFVFFTVFSAVYFMTVVGNLLIVVIVTSDPHLHTTMY
NOV25a FLLGNLSFLDFCYSSITAPRMLVDLLSGNPTISFGGCLTQLFFFHFIGGIKIFLLTVMAY
NOV25b FLLGNLSFLDFCYSSITAPRMLVDLLSGNPTISFGGCLTQLFFFHFIGGIKIFLLTVMAY
NOV25c FLLGNLSFLDFCYSSITAPRMLVDLLSGNPTISFGGCLTQLFFFHFIGGIKIFLLTVMAY
NOV25a DRYIAISQPLHYTLIMNQTVCALLMAASWVGGFINSIVQIALTIQLPFCGPDKLDNFYCD
NOV25b DRYIAISQPLHYTLIMNQTVCALLMAASWVGGFINSIVQIALTIQLPFCGPDKLDNFYCD
NOV25c DRYIAISQPLHYTLIMNQTVCALLMAASWVGGFINSIVQIALTIQLPFCGPDKLDNFYCD
NOV25a VPQLIKLACTDTFVLELLMVSNNGLVTLMCFLVLLGSYTALLVMLRSHSREGRSKALSTC
NOV25b VPQLIKLACTDTFVLELLMVSNNGLVTLMCFLVLLGSYTALLVMLRSHSREGRSKALSTC
NOV25c VPQLIKLACTDTFVLELLMVSNNGLVTLMCFLVLLGSYTALLVMLRSHSREGRSKALSTC
NOV25a ASHIAVVTLIFVPCIYVYTRPFRTFPMDKAVSVLYTIVTPMLNPAIYTLRNKEVIMAMKK
NOV25b ASHIAVVTLIFVPCIYVYTRPFRTFPMDKAVSVLYTIVTPMLNPAIYTLRNKEVIMAMKK
NOV25c ASHIAVVTLIFVPCIYVYTRPFRTFPMDKAVSVLYTIVTPMLNPAIYTLRNKEVIMAMKK
NOV25a LWRRKKDPIGPLEHRPLH
NOV25b LWRRKKDPIGPLENRPLH
NOV25c LWRRKKDPIGPLEHRPLH
NOV25a (SEQ ID NO: 324)
NOV25b (SEQ ID NO: 326)
NOV25C (SEQ ID NO: 328)

Further analysis of the NOV25a protein yielded the following properties shown in Table 25C.

TABLE 25C
Protein Sequence Properties NOV25a

| | |
|---|---|
| SignalP analysis: | Cleavage site between residues 52 and 53 |
| PSORT II analysis: | |

| | |
|---|---|
| PSG: | a new signal peptide prediction method |
| | N-region: length 0; pos.chg 0; neg.chg 0 |
| | H-region: length 21; peak value 9.57 |
| | PSG score: 5.17 |
| GvH: | von Heijne's method for signal seq. recognition |
| | GvH score (threshold: −2.1): −5.51 |
| | possible cleavage site: between 32 and 33 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 6 | | |
| | INTEGRAL | Likelihood = −8.23 | Transmembrane 33-49 |
| | INTEGRAL | Likelihood = −4.04 | Transmembrane 101-117 |
| | INTEGRAL | Likelihood = −0.90 | Transmembrane 142-158 |
| | INTEGRAL | Likelihood = −0.90 | Transmembrane 184-200 |
| | INTEGRAL | Likelihood = −6.37 | Transmembrane 208-224 |
| | INTEGRAL | Likelihood = −4.51 | Transmembrane 241-257 |

| | |
|---|---|
| PERIPHERAL | Likelihood = 0.79 (at 12) |

| | |
|---|---|
| | ALOM score: −8.23 (number of TMSs: 6) |
| MTOP: | Prediction of membrane topology (Hartmann et al.) |
| | Center position for calculation: 40 |
| | Charge difference: 0.0 C(0.0) − N(0.0) |
| | N >= C: N-terminal side will be inside |

>>> membrane topology: type 3a
MITDISC: discrimination of mitochondrial targeting seq

| | | | |
|---|---|---|---|
| R content: | 0 | Hyd Moment(75): | 1.55 |
| Hyd Moment(95): | 4.07 | G content: | 2 |
| D/E content: | 1 | S/T content: | 2 |

Score: −6.22

Gavel: prediction of cleavage sites for mitochondrial preseq

R-2 motif at 132 DRY|IA

NUCDISC: discrimination of nuclear localization signals

pat4: RRKK (5) at 303
pat7: none
bipartite: none
content of basic residues: 6.9%
NLS Score: −0.16

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

66.7%: endoplasmic reticulum
22.2%: mitochondrial
11.1%: nuclear

>> prediction for CG53530-03 is end (k = 9)

A search of the NOV25a protein against the Geneseq database, a proprietary database that contains sequences published in patents and patent publication, yielded several homologous proteins shown in Table 25D.

TABLE 25D
Geneseq Results for NOV25a

| | | NOV25a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85175 | G-coupled olfactory receptor #36 - | 1 . . . 318 | 318/318 (100%) | 0.0 |
| | Homo sapiens, 318 aa. | 1 . . . 318 | 318/318 (100%) | |
| | [WO200198526-A2, 27-DEC-2001] | | | |
| AAU95525 | Human olfactory and pheromone | 1 . . . 318 | 318/318 (100%) | 0.0 |
| | G protein-coupled receptor #12 - | 1 . . . 318 | 318/318 (100%) | |
| | Homo sapiens, 318 aa. | | | |
| | [WO200224726-A2, 28-MAR-2002] | | | |
| AAU05140 | Human odorant receptor (OR)- | 1 . . . 318 | 318/318 (100%) | 0.0 |
| | like protein, NOV10 - Homo | 1 . . . 318 | 318/318 (100%) | |
| | sapiens, 318 aa. | | | |
| | [WO200151632-A2, 19-JUL-2001] | | | |
| AAU10320 | G-protein coupled receptor | 1 . . . 318 | 318/318 (100%) | 0.0 |
| | (GCREC) #21 - Homo sapiens, | 1 . . . 318 | 318/318 (100%) | |
| | 318 aa. [WO200166742-A2, 13-SEP-2001] | | | |
| AAG71624 | Human olfactory receptor | 1 . . . 315 | 314/315 (99%) | 0.0 |
| | polypeptide, SEQ ID NO: 1305 - | 1 . . . 315 | 314/315 (99%) | |
| | Homo sapiens, 315 aa. | | | |
| | [WO200127158-A2, 19-APR-2001] | | | |

In a BLAST search of public sequence databases, the NOV25a protein was found to have homology to the proteins shown in the BLASTP data in Table 25E.

TABLE 25E
Public BLASTP Results for NOV25a

| | | NOV25a | Identities/ | |
|---|---|---|---|---|
| Protein | | Residues/ | Similarities | |
| Accession | | Match | for the Matched | |
| Number | Protein/Organism/Length | Residues | Portion | Expect Value |
| Q8NGN0 | Seven transmembrane helix | 1 . . . 318 | 318/318 (100%) | 0.0 |
| | receptor - Homo sapiens | 1 . . . 318 | 318/318 (100%) | |
| | (Human), 318 aa. | | | |
| Q8VFN1 | Olfactory receptor MOR239-6 - | 1 . . . 314 | 284/314 (90%) | e−166 |
| | Mus musculus (Mouse), 314 aa. | 1 . . . 314 | 296/314 (93%) | |
| Q8VFU8 | Olfactory receptor MOR239-5 - | 1 . . . 317 | 200/317 (63%) | e−113 |
| | Mus musculus (Mouse), 314 aa. | 1 . . . 313 | 241/317 (75%) | |
| Q8NGE8 | Seven transmembrane helix | 1 . . . 304 | 190/304 (62%) | e−110 |
| | receptor - Homo sapiens | 1 . . . 304 | 236/304 (77%) | |
| | (Human), 314 aa. | | | |
| Q8VF58 | Olfactory receptor MOR240-1 - | 1 . . . 304 | 186/304 (61%) | e−110 |
| | Mus musculus (Mouse), 311 aa. | 1 . . . 304 | 237/304 (77%) | |

PFam analysis indicates that the NOV25a protein contains the domains shown in the Table 25F.

TABLE 25F
Domain Analysis of NOV25a

| | | Identities/ | |
|---|---|---|---|
| | NOV25a Match | Similarities for | |
| Pfam Domain | Region | the Matched Region | Expect Value |
| 7tm_1 | 41 . . . 287 | 57/276 (21%) | 3.3e−28 |
| | | 172/276 (62%) | |

### Example 26

The NOV26 clone was analyzed, and the nucleotide and encoded polypeptide sequences are shown in Table 26A.

TABLE 26A
NOV26 Sequence Analysis

| | | |
|---|---|---|
| NOV26a, CG53563-03 | SEQ ID NO: 329 | 1028 bp |
| DNA Sequence | ORF Start: ATG at 28 | ORF Stop: TAG at 961 |

AGGGAGAGAGACCAAGGGTGAGAAGAAATGTCCAACGCCAGCCTACTGACAGCGTTCATCCTCATGGG
CCTTCCCCATGCCCCAGCGCTGGACGCCCCCCTCTTTGGAGTCTTCCTGGTGGTTTACGTGCTCACTG
TGCTGGGGAACCTCCTCATCCTGCTGGTGATCAGGGTGGATTCTCACCTCCACACCACCATGTACTAC
TTCCTCACCAACCTGTCGTTCATTGACATGTGGTTCTCCACTGTCACGGTGCCCAAATTGCTGATGAC
TTTGGTGTTCCCAAGTGGCAGGGCTATCTCCTTCCACAGCTGCATGGCTCAGCTCTATTTCTTTCACT
TCCTAGGGGGCACCGAGTGTTTCCTCTACAGGGTCATGTCCTGTGATCGCTACCTGGCCATCAGTTAC
CCGCTCAGGTACACCAGCATGATGACTGGGCGCTCGTGTACTCTTCTGGCCACCAGCACTTGGCTCAG
TGGCTCTCTGCACTCTGCTGTCCAGGCCATATTGACTTTCCATTTGCCCTACTGTGGACCCAACTGGA
TCCAGCACTATTTGTGTGATGCACCGCCCATCCTGAAACTGGCCTGTGCAGACACCTCAGCCATAGAG
ACTGTCATTTTTGTGACTGTTGGAATAGTGGCCTCGGGCTGCTTTGTCCTGATAGTGCTGTCCTATGT
GTCCATCGTCTGTTCCATCCTGCGGATCCGCACCTCAGAGGGGAAGCACAGAGCCTTTCAGACCTGTG
CCTCCCACTGTATCGTGGTCCTTTGCTTCTTTGGCCCTGGTCTTTTCATTTACCTGAGGCCAGGCTCC
AGGAAAGCTGTGGATGGAGTTGTGGCCGTTTTCTACACTGTGCTGACGCCCCTTCTCAACCCTGTTGT
GTACACCCTGAGGAACAAGGAGGTGAAGAAAGCTCTGTTGAAGCTGAAAGACAAAGTAGCACATTCTC
AGAGCAAATAGACACTAGGGAAGATTACATATCTTAGCTCTTGTGAATAGTGCTGTGAAAAACATACA
GGGGCAGG

| | | | |
|---|---|---|---|
| NOV26a, GG53563-03 | SEQ ID NO: 330 | 311 aa | MW at 34518.7kD |
| Protein Sequence | | | |

MSNASLLTAFILMGLPHAPALDAPLFGVFLVVYVLTVLGNLLILLVIRVDSHLHTTMYYFLTNLSFID
MWFSTVTVPKLLMTLVFPSGRAISFHSCMAQLYFFHFLGGTECFLYRVMSCDRYLAISYPLRYTSMMT
GRSCTLLATSTWLSGSLHSAVQAILTFHLPYCGPNWIQHYLCDAPPILKLACADTSAIETVIFVTVGI
VASGCFVLIVLSYVSIVCSILRIRTSEGKHRAFQTCASHCIVVLCFFGPGLFIYLRPGSRKAVDGVVA
VFYTVLTPLLNPVVYTLRNKEVKKALLKLKDKVAHSQSK

| | | |
|---|---|---|
| NOV26b, CG53563-01 | SEQ ID NO: 331 | 1038 bp |
| DNA Sequence | ORF Start: ATG at 28 | ORF Stop: TAG at 961 |

AGGGAGAGAGACCAAGGGTGAGAAGAAATGTCCAACGCCAGCCTACTGACAGCGTTCATCCTCATGGG
CCTTCCCCATGCCCCAGCGCTGGACGCCCCCCTCTTTGGAGTCTTCCTGGTGGTTTACGTGCTCACTG
TGCTGGGGAACCTCCTCATCCTGCTGGTGATCAGGGTGGATTCTCACCTCCACACCACCATGTACTAC
TTCCTCACCAACCTGTCGTTCATTGACATGTGGTTCTCCACTGTCACGGTGCCCAAATTGCTGATGAC
TTTGGTGTTCCCAAGTGGCAGGGCTATCTCCTTCCACAGCTGCATGGCTCAGCTCTATTTCTTTCACT
TCCTAGGGGGCACCGAGTGTTTCCTCTACAGGGTCATGTCCTGTGATCGCTACCTGGCCATCAGTTAC
CCGCTCAGGTACACCAGCATGATGACTGGGCGCTCGTGTACTCTTCTGGCCACCAGCACTTGGCTCAG
TGGCTCTCTGCACTCTGCTGTCCAGGCCATATTGACTTTCCATTTGCCCTACTGTGGACCCAACTGGA
TCCAGCACTATTTGTGTGATGCACCGCCCATCCTGAAACTGGCCTGTGCAGACACCTCAGCCATAGAG
ACTGTCATTTTTGTGACTGTTGGAATAGTGGCCTCGGGCTGCTTTGTCCTGATAGTGCTGTCCTATGT
GTCCATCGTCTGTTCCATCCTGCGGATCCGCACCTCAGAGGGGAAGCACAGAGCCTTTCAGACCTGTG
CCTCCCACTGTATCGTGGTCCTTTGCTTCTTTGGCCCTGGTCTTTTCATTTACCTGAGGCCAGGCTCC
AGGAAAGCTGTGGATGGAGTTGTGGCCGTTTTCTACACTGTGCTGACGCCCCTTCTCAACCCTGTTGT
GTACACCCTGAGGAACAAGGAGGTGAAGAAAGCTCTGTTGAAGCTGAAAGACAAAGTAGCACATTCTC
AGAGCAAATAGACACTAGGGAAGATTACATATCTTAGCTCTTGTGAATAGTGCTGTGAAAAACATACA
GGGGCAGGTATCTTTTGG

| | | | |
|---|---|---|---|
| NOV26b, GG53563-01 | SEQ ID NO: 332 | 311 aa | MW at 34518.7kD |
| Protein Sequence | | | |

MSNASLLTAFILMGLPHAPALDAPLFGVFLVVYVLTVLGNLLILLVIRVDSHLHTTMYYFLTNLSFID
MWFSTVTVPKLLMTLVFPSGRAISFHSCMAQLYFFHFLGGTECFLYRVMSCDRYLAISYPLRYTSMMT
GRSCTLLATSTWLSGSLHSAVQAILTFHLPYCGPNWIQHYLCDAPPILKLACADTSAIETVIFVTVGI
VASGCFVLIVLSYVSIVCSILRIRTSEGKHRAFQTCASHCIVVLCFFGPGLFIYLRPGSRKAVDGVVA
VFYTVLTPLLNPVVYTLRNKEVKKALLKLKDKVAHSQSK

| | | |
|---|---|---|
| NOV26c, CG53563-02 | SEQ ID NO: 333 | 1040 bp |
| DNA Sequence | ORF Start: ATG at 36 | ORF Stop: TAG at 969 |

TTTCTGTAAGAACAGAGCCCCATATATGAGAAGAAATGTCCAACGCCACCCTACTGACAGCGTTCATC
CTCACGGGCCTTCCCCATGCCCCAGGGCTGGACGCCCCCCTCTTTGGAATCTTCCTGGTGGTTTACGT
GCTCACTGTGCTGGGGAACCTCCTCATCCTGCTGGTGATCAGGGTGGATTCTCACCTCCACACCCCCA
TGTACTACTTCCTCACCAACCTGTCCTTCATTGACATGTGGTTCTCCACTGTCACGGTGCCCAAAATG
CTGATGACCTTGGTGTCCCCAAGCGGCAGGACTATCTCCTTCCACAGCTGCGTGGCTCAGCTCTATTT
TTTCCACTTCCTGGGGAGCACCGAGTGTTTCCTCTACACAGTCATGTCCTATGATCGCTACCTGGCCA
TCAGTTACCCGCTCAGGTACACCAACATGATGACTGGGCGCTCGTGTGCCCTCCTGGCCACCGGCACT
TGGCTCAGTGGCTCTCTGCACTCTGCTGTCCAGACCATATTGACTTTCCATTTGCCCTACTGTGGACC
CAACCAGATCCAGCACTACTTCTGTGACGCACCGCCCATCCTGAAACTGGCCTGTGCAGACACCTCAG
CCAACGAGATGGTCATCTTTGTGAATATTGGGCTAGTGGCCTCGGGCTGCTTTGTCCTGATAGTGCTG
TCCTATGTGTCCATCGTCTGTTCCATCCTGCGGATCCGCACCTCAGAGGGGAGGCAGAGAGCCTTTCA
GACCTGTGCCTCCCACTGTATCGTGGTCCTTTGCTTCTTTGGCCCTGGTCTTTTCATTTACCTGAGGC
CAGGCTCCAGGGACGCCTTGCATGGGGTTGTGGCCGTTTTCTACACCACGCTGACTCCTCTTTTCAAC
CCTGTTGTGTACACCCTGAGAAACAAGGAGGTAAAGAAAGCTCTGTTGAAGCTGAAAAATGGGTCAGT
ATTTGCTCAGGGTGAATAGTTAAGAAAGGCCATATATGGCTAACTTTTCTTTTTTTATTTGTAATTAA
ATTAAACCTTCAACATAAGC

| | | | |
|---|---|---|---|
| NOV26c, CG53563-02 | SEQ ID NO: 334 | 311 aa | MW at 34516.4kD |
| Protein Sequence | | | |

MSNATLLTAFILTGLPHAPGLDAPLFGIFLVVYVLTVLGNLLILLVIRVDSHLHTPMYYFLTNLSFID
MWFSTVTVPKMLMTLVSPSGRTISFHSCVAQLYFFHFLGSTECFLYTVMSYDRYLAISYPLRYTNMMT
GRSCALLATGTWLSGSLHSAVQTILTFHLPYCGPNQIQHYFCDAPPILKLACADTSANEMVIFVNIGL
VASGCFVLIVLSYVSIVCSILRIRTSEGRHRAFQTCASHCIVVLCFFGPGLFIYLRPGSRDALHGVVA
VFYTTLTPLFNPVVYTLRNKEVKKALLKLKNGSVFAQGE

A ClustalW comparison of the above protein sequences yields the following sequence alignment shown in Table 26B.

TABLE 26B
Comparison of the NOV26 protein sequences.

NOV26a MSNASLLTAFILMGLPHAPALDAPLFGVFLVVYVLTVLGNLLILLVIRVDSHLHTTMYYF
NOV26b MSNASLLTAFILMGLPHAPALDAPLFGVFLVVYVLTVLGNLLILLVIRVDSHLHTTMYYF
NOV26c MSNATLLTAFILTGLPHAPGLDAPLFGIFLVVYVLTVLGNLLILLVIRVDSHLHTTMYYF
NOV26a LTNLSFIDMWFSTVTVPKLLMTLVFPSGRAISFHSCMAQLYFFHFLGGTECFLYRVMSCD
NOV26b LTNLSFIDMWFSTVTVPKLLMTLVFPSGRAISFHSCMAQLYFFHFLGGTECFLYRVMSCD
NOV26c LTNLSFIDMWFSTVTVPKMLMTLVSPSGRTISFHSCVAQLYFFHFLGSTECFLYRVMSYD
NOV26a RYLAISYPLRYTSMMTGRSCTLLATSTWLSGSLHSAVQAILTFHLPYCGPNWIQHYLCDA
NOV26b RYLAISYPLRYTSMMTGRSCTLLATSTWLSGSLHSAVQAILTFHLPYCGPNWIQHYLCDA
NOV26c RYLAISYPLRYTNMMTGRSCALLATGTWLSGSLHSAVQTILTFHLPYCGPNQIQHYFCDA
NOV26a PPILKLACADTSAIETVIFVTVGIVASGCFVLIVLSYVSIVCSILRIRTSEGKHRAFQTC
NOV26b PPILKLACADTSAIETVIFVTVGIVASGCFVLIVLSYVSIVCSILRIRTSEGKHRAFQTC
NOV26c PPILKLACADTSANEMVIFVNIGLVASGCFVLIVLSYVSIVCSILRIRTSEGRHRAFQTC
NOV26a ASHCIVVLCFFGPGLFIYLRPGSRKAVDGVVAVFYTVLTPLLNPVVYTLRNKEVKKALLK
NOV26b ASHCIVVLCFFGPGLFIYLRPGSRKAVDGVVAVFYTVLTPLLNPVVYTLRNKEVKKALLK
NOV26c ASHCIVVLCFFGPGLFIYLRPGSRDALHGVVAVFYTTLTPLFNPVVYTLRNKEVKKALLK
NOV26a LKDKVAHSQSK
NOV26b LKDKVAHSQSK
NOV26c LKNGSVFAQGE
NOV26a (SEQ ID NO: 330)
NOV26b (SEQ ID NO: 332)
NOV26c (SEQ ID NO: 334)

Further analysis of the NOV26a protein yielded the following properties shown in Table 26C.

TABLE 26C
Protein Sequence Properties NOV26a

| | |
|---|---|
| SignalP analysis: | Cleavage site between residues 52 and 53 |
| PSORT II analysis: | |

| | |
|---|---|
| PSG: | a new signal peptide prediction method |
| | N-region: length 0; pos.chg 0; neg.chg 0 |
| | H-region: length 21; peak value 11.19 |
| | PSG score: 6.79 |
| GvH: | von Heijne's method for signal seq. recognition |
| | GvH score (threshold: −2.1): −3.58 |
| | possible cleavage site: between 51 and 52 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 4 | | |
| | INTEGRAL | Likelihood = −10.61 | Transmembrane 31-47 |
| | INTEGRAL | Likelihood = −10.08 | Transmembrane 197-213 |
| | INTEGRAL | Likelihood = −4.46 | Transmembrane 243-259 |
| | INTEGRAL | Likelihood = −2.60 | Transmembrane 270-286 |

| | |
|---|---|
| PERIPHERAL | Likelihood = 0.85 (at 5) |

| | |
|---|---|
| | ALOM score: −10.61 (number of TMSs: 4) |
| MTOP: | Prediction of membrane topology (Hartmann et al.) |
| | Center position for calculation: 38 |
| | Charge difference: 1.5 C(1.0) − N(−0.5) |
| | C > N: C-terminal side will be inside |

>>>Caution: Inconsistent mtop result with signal peptide
>>> membrane topology: type 3b
MITDISC: discrimination of mitochondrial targeting seq

| | | | |
|---|---|---|---|
| R content: | 0 | Hyd Moment(75): | 4.41 |
| Hyd Moment(95): | 3.21 | G content: | 1 |
| D/E content: | 1 | S/T content: | 3 |

Score: −5.14

Gavel: prediction of cleavage sites for mitochondrial preseq

R-2 motif at 99 GRA|IS

NUCDISC: discrimination of nuclear localization signals

pat4: none
pat7: none
bipartite: none
content of basic residues: 7.4%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG53563-03 is end (k = 9)

A search of the NOV26a protein against the Geneseq database, a proprietary database that contains sequences published in patents and patent publication, yielded several homologous proteins shown in Table 26D.

TABLE 26D
Geneseq Results for NOV26a

| | | NOV26a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85273 | G-coupled olfactory receptor | 1 . . . 311 | 311/311 (100%) | 0.0 |
| | #134 - Homo sapiens, 311 aa. | 1 . . . 311 | 311/311 (100%) | |
| | [WO200198526-A2, 27-DEC- | | | |
| | 2001] | | | |
| AAU95627 | Human olfactory and pheromone | 1 . . . 311 | 311/311 (100%) | 0.0 |
| | G protein-coupled receptor #114 - | 1 . . . 311 | 311/311 (100%) | |
| | Homo sapiens, 311 aa. | | | |
| | [WO200224726-A2, 28-MAR- | | | |
| | 2002] | | | |
| ABP95802 | Human GPCR polypeptide SEQ | 1 . . . 311 | 311/311 (100%) | 0.0 |
| | ID NO 414 - Homo sapiens, 311 | 1 . . . 311 | 311/311 (100%) | |
| | aa. [WO200216548-A2, 28-FEB- | | | |
| | 2002] | | | |
| AAG66376 | Partial NOV 15 protein sequence | 1 . . . 311 | 311/311 (100%) | 0.0 |
| | #2 - Unidentified, 311 aa. | 1 . . . 311 | 311/311 (100%) | |
| | [WO200155179-A2, 02-AUG- | | | |
| | 2001] | | | |
| AAG66335 | Human NOV 15 protein sequence - | 1 . . . 311 | 311/311 (100%) | 0.0 |
| | Homo sapiens, 311 aa. | 1 . . . 311 | 311/311 (100%) | |
| | [WO200155179-A2, 02-AUG- | | | |
| | 2001] | | | |

In a BLAST search of public sequence databases, the NOV26a protein was found to have homology to the proteins shown in the BLASTP data in Table 26E.

TABLE 26E
Public BLASTP Results for NOV26a

| | | NOV26a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8NGN5 | Seven transmembrane helix | 1 . . . 311 | 311/311 (100%) | 0.0 |
| | receptor - Homo sapiens | 1 . . . 311 | 311/311 (100%) | |
| | (Human), 311 aa. | | | |
| CAC69628 | Sequence 13 from Patent | 1 . . . 311 | 308/311 (99%) | e−179 |
| | WO0155179 - Homo sapiens | 1 . . . 311 | 308/311 (99%) | |
| | (Human), 311 aa. | | | |
| CAC69627 | Sequence 11 from Patent | 1 . . . 311 | 308/311 (99%) | e−178 |
| | WO0155179 - Homo sapiens | 1 . . . 311 | 308/311 (99%) | |
| | (Human), 311 aa. | | | |
| Q8NGN4 | Seven transmembrane helix | 1 . . . 311 | 274/311 (88%) | e−159 |
| | receptor - Homo sapiens | 1 . . . 311 | 288/311 (92%) | |
| | (Human), 311 aa. | | | |
| Q8NGN6 | Seven transmembrane helix | 1 . . . 311 | 274/311 (88%) | e−159 |
| | receptor - Homo sapiens | 1 . . . 311 | 286/311 (91%) | |
| | (Human), 311 aa. | | | |

PFam analysis indicates that the NOV26a protein contains the domains shown in the Table 26F.

TABLE 26F
Domain Analysis of NOV26a

| | | Identities/ | |
|---|---|---|---|
| | NOV26a Match | Similarities for | |
| Pfam Domain | Region | the Matched Region | Expect Value |
| 7tm_1 | 39 . . . 287 | 52/276 (19%) | 2.4e−26 |
| | | 165/276 (60%) | |

### Example 27

The NOV27 clone was analyzed, and the nucleotide and encoded polypeptide sequences are shown in Table 27A.

TABLE 27A
NOV27 Sequence Analysis

| | | |
|---|---|---|
| NOV27a, GG53719-02 | SEQ ID NO: 335 | 993 bp |
| DNA Sequence | ORF Start: at 256 | ORF Stop: end of sequence |

TATTAGAAGAAGAGTGTCATAGAAGCCAATAACATTTCTGGGCCTGTGAGTGAATTTATCCTCCTGGG
CTTCCCCTGCCTGCTGCAGGGAGACCAAGATCCTCCTCTTTGTGGTCTTCTCCCTCATCTACCTTCTG
ACCCTCATGGGTAACACATCCATCATCTGCGCTGTGTGGTCAAGCCAGAAACTCCACACACCTATGTA
CATCCTCTTGGCTAATTTCTCTTTCCTGGAGATCTGCTGCATTAGTTCTGATGTCCCAAAATGTTGGC
CAATCTCATCTCCCATATCAAGAGCATCTCCTATGCTGGCTGCCTGCTCCAGTTCTTCTACTTCTCCA
TGTGTGCTGCAGAGGGCTATTTTCTGTCTGTGATGTCCTTTGATCGGTTCCTTACCATCTGTCGACCT
TTGCATTATCCCACAGTCATGACTCACCACCTGTGTGTCCGATTAGTGGCCTTCTGCAGGGCAGGTGG
TTTTCTATCCATACTGATGCCTGCAGTGCTTATGTCCCGAGTGCCTTTCTGTGGCCCTAACATCACTG
ACCATTTTTTCTGTAACCTGGGACCATTGCTGGCACTGTCCTGTGCCCCAGTTCCCAAAACTACTCTG
ACTTGTGCTACAGTAAGCTCTCTCATCATCTTCATCACCTTCCTCTACATTCTTGGGTCCCATATCTT
AGTTTTGCGAGCTGTTCTGTGGGTCCCAGCTGGCTCAGGCAGGAACAAAGCTTTCTCTACATGTGCTT
CCCATTTCTTGGTTGTTTCTTTCTTCTATGGCTCAGTCATGGTGATGTATGTGAGTCCAGGCTCCAGG
AGCCGTCCTGGGACACAGAAATTTGTGACATTGTTTTACTGCACAGCAAACCCATTCTTTAATCCCCT
GACCTACAGTCTCTGGAACAAAGATATGACAGATGCCCTTAAAAAAGTGCTGGGAGTGCCATCAAAAG
AAATATCTTGGAACACACTGAAATGATATACATTCTTCTAC

| | | | |
|---|---|---|---|
| NOV27a, GG53719-02 | SEQ ID NO: 336 | 321 aa | MW at 35567.8kD |
| Protein Sequence | | | |

MSVIEANNISGPVSELSSWASPACCRETKILLFVVFSLIYLLTLMGNTSIICAVWSSQKLHTPMYILL
ANFSFLEICCISSDVPMLANLISHIKSISYAGCLLQFFYFSMCAAEGYFLSVMSFDRFLTICRPLHYP
TVMTHHLCVRLVAFCRAGGFLSILMPAVLMSRVPFCGPNITDHFFCNLGPLLALSCAPVPKTTLTCAT
VSSLIIFITFLYILGSHILVLRAVLWVPAGSGRNKAFSTCASHFLVVSFFYGSVMVMYVSPGSRSRPG
TQKFVTLFYCTANPFFNPLTYSLWNKDMTDALKKVLGVPSKEISWNTLK

| | | |
|---|---|---|
| NOV27b, CG53719-01 | SEQ ID NO: 337 | 1012 bp |
| DNA Sequence | ORF Start: ATG at 25 | ORF Stop: TGA at 988 |

TCATTTCCTTCATAGATTAGAAGAATGAGTGTCATAGAAGCCAATAACATTTCTGGGCCTGTGAGTGA
ATTTATCCTCCTGGGCTTCCCTGCCTGCTGCAGGGAGACCAAGATCCTCCTCTTTGTGGTCTTCTCCC
TCATCTACCTTCTGACCCTCATGGGTAACACATCCATCATCTGCGCTGTGTGGTCAAGCCAGAAACTC
CACACACCTATGTACATCCTCTTGGCTAATTTCTCTTTCCTGGAGATCTGCTGCATTAGTTCTGATGT
CCCAATGTTGGCCAATCTCATCTCCCATATCAAGAGCATCTCCTATGCTGGCTGCCTGCTCCAGTTCT
TCTACTTCTCCATGTGTGCTGCAGAAGGCTACTTTCTGTCTGTGATGTCCTTTGATCGGTTCCTTACC
ATCTGTCGACCTTTGCATTATCCCACAGTCATGACTCACCACCTGTGTGTCCGATTAGTGGCCTTCTG
CAGGGCAGGTGGTTTTCTATCCATACTGATGCCTGCAGTGCTTATGTCCCGAGTGCCTTTCTGTGGCC
CTAACATCACTGACCATTTTTTCTGTAACCTGGGACCATTGCTGGCACTGTCCTGTGCCCCAGTTCCC
AAAACTACTCTGACTTGTGCTACAGTAAGCTCTCTCATCATCTTCATCACCTTCCTCTACATTCTTGG
GTCCCATATCTTAGTTTTGCGAGCTGTTCTGTGGGTCCCAGCTGGCTCAGGCAGGAACAAAGCTTTCT
CTACATGTGCTTCCCATTTCTTGGTTGTTTCTTTCTTCTATGGCTCAGTCATGGTGATGTATGTGAGT
CCAGGCTCCAGGAGCCGCCCTGGGACACAGAAATTTGTGACATTGTTTTACTGCACAGCAACCCCATT
CTTTAATCCCCTGACCTACAGTCTCTGGAACAAAGATATGACAGATGCCCTTAAAAAAGTGCTGGGAG
TGCCATCAAAAGAAATATATTGGAACACACTGAAATGATATACATTCTTCTACAATTATT

| | | | |
|---|---|---|---|
| NOV27b, CG53719-01 | SEQ ID NO: 338 | 321 aa | MW at 35690.1kD |
| Protein Sequence | | | |

MSVIEANNISGPVSEFILLGFPACCRETKILLFVVFSLIYLLTLMGNTSIICAVWSSQKLHTPMYILL
ANFSFLEICCISSDVPMLANLISHIKSISYAGCLLQFFYFSMCAAEGYFLSVMSFDRFLTICRPLHYP
TVMTHHLCVRLVAFCRAGGFLSILMPAVLMSRVPFCGPNITDHFFCNLGPLLALSCAPVPKTTLTCAT
VSSLIIFITFLYILGSHILVLRAVLWVPAGSGRNKAFSTCASHFLVVSFFYGSVMVMYVSPGSRSRPG
TQKFVTLFYCTATPFFNPLTYSLWNKDMTDALKKVLGVPSKEIYWNTLK

A ClustalW comparison of the above protein sequences yields the following sequence alignment shown in Table 27B.

TABLE 27B
Comparison of the NOV27 protein sequences.

NOV27a MSVIEANNISGPVSELSSWASPACCRETKILLFVVFSLIYLLTLMGNTSIICAVWSSQKL
NOV27b MSVIEANNISGPVSEFILLGFPACCRETKILLFVVFSLIYLLTLMGNTSIICAVWSSQKL
NOV27a HTPMYILLANFSFLEICCISSDVPMLANLISHIKSISYAGCLLQFFYFSMCAAEGYFLSV
NOV27b HTPMYILLANFSFLEICCISSDVPMLANLISHIKSISYAGCLLQFFYFSMCAAEGYFLSV
NOV27a MSFDRFLTICRPLHYPTVMTHHLCVRLVAFCRAGGFLSILMPAVLMSRVPFCGPNITDHF
NOV27b MSFDRFLTICRPLHYPTVMTHHLCVRLVAFCRAGGFLSILMPAVLMSRVPFCGPNITDHF
NOV27a FCNLGPLLALSCAPVPKTTLTCATVSSLIIFITFLYILGSHILVLRAVLWVPAGSGRNKA
NOV27b FCNLGPLLALSCAPVPKTTLTCATVSSLIIFITFLYILGSHILVLRAVLWVPAGSGRNKA
NOV27a FSTCASHFLVVSFFYGSVMVMYVSPGSRSRPGTQKFVTLFYCTANPFFNPLTYSLWNKDM
NOV27b FSTCASHFLVVSFFYGSVMVMYVSPGSRSRPGTQKFVTLFYCTATPFFNPLTYSLWNKDM
NOV27a TDALKKVLGVPSKEISWNTLK
NOV27b TDALKKVLGVPSKEIYWNTLK
NOV27a (SEQ ID NO: 336)
NOV27b (SEQ ID NO: 338)

Further analysis of the NOV27a protein yielded the following properties shown in Table 27C.

TABLE 27C
Protein Sequence Properties NOV27a

| | |
|---|---|
| SignalP analysis: | Cleavage site between residues 47 and 48 |
| PSORT II analysis: | |

| | |
|---|---|
| PSG: | a new signal peptide prediction method |
| | N-region: length 5; pos.chg 0; neg.chg 1 |
| | H-region: length 9; peak value 0.00 |
| | PSG score: −4.40 |
| GvH: | von Heijne's method for signal seq. recognition |
| | GvH score (threshold: −2.1): −3.31 |
| | possible cleavage site: between 43 and 44 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood = −9.34 | Transmembrane 30-46 |
| | INTEGRAL | Likelihood = −1.28 | Transmembrane 64-80 |
| | INTEGRAL | Likelihood = −0.96 | Transmembrane 156-172 |
| | INTEGRAL | Likelihood = −7.54 | Transmembrane 208-224 |
| | INTEGRAL | Likelihood = −2.50 | Transmembrane 248-264 |

| | |
|---|---|
| PERIPHERAL | Likelihood = 1.48 (at 180) |

| | |
|---|---|
| | ALOM score: −9.34 (number of TMSs: 5) |
| MTOP: | Prediction of membrane topology (Hartmann et al.) |
| | Center position for calculation: 37 |
| | Charge difference: 1.5 C(1.5) − N(0.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 0.86 |
| Hyd Moment(95): | 3.03 | G content: | 1 |
| D/E content: | 2 | S/T content: | 3 |

Score: −7.70

Gavel: prediction of cleavage sites for mitochondrial preseq

cleavage site motif not found

NUCDISC: discrimination of nuclear localization signals

pat4: none
pat7: none
bipartite: none
content of basic residues: 6.5%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG53719-02 is end (k = 9)

A search of the NOV27a protein against the Geneseq database, a proprietary database that contains sequences published in patents and patent publication, yielded several homologous proteins shown in Table 27D.

TABLE 27D
Geneseq Results for NOV27a

| | | NOV27a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU07092 | Human odorant receptor (OR) | 1 . . . 321 | 313/321 (97%) | 0.0 |
| | polypeptide #9 - Homo sapiens, | 1 . . . 321 | 313/321 (97%) | |
| | 321 aa. [WO200157215-A2, 09- | | | |
| | AUG-2001] | | | |
| ABP72226 | Human G-protein coupled | 1 . . . 321 | 314/322 (97%) | 0.0 |
| | receptor GCREC-20 (odorant | 1 . . . 322 | 314/322 (97%) | |
| | receptor) - Homo sapiens, 322 aa. | | | |
| | [WO2003000859-A2, 03-JAN- | | | |
| | 2003] | | | |
| AAG72445 | Human OR-like polypeptide query | 1 . . . 321 | 310/322 (96%) | 0.0 |
| | sequence, SEQ ID NO: 2126 - | 1 . . . 322 | 311/322 (96%) | |
| | Homo sapiens, 322 aa. | | | |
| | [WO200127158-A2, 19-APR- | | | |
| | 2001] | | | |
| AAG72079 | Human olfactory receptor | 1 . . . 321 | 309/322 (95%) | e−180 |
| | polypeptide, SEQ ID NO: 1760 - | 1 . . . 322 | 310/322 (95%) | |
| | Homo sapiens, 322 aa. | | | |
| | [WO200127158-A2, 19-APR- | | | |
| | 2001] | | | |
| ABP72218 | Human G-protein coupled | 29 . . . 321 | 255/295 (86%) | e−149 |
| | receptor GCREC-12 (olfactory | 39 . . . 333 | 270/295 (91%) | |
| | receptor) - Homo sapiens, 333 aa. | | | |
| | [WO2003000859-A2, 03-JAN- | | | |
| | 2003] | | | |

In a BLAST search of public sequence databases, the NOV27a protein was found to have homology to the proteins shown in the BLASTP data in Table 27E.

TABLE 27E
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8VFT6 | Olfactory receptor MOR106-5 - | 6 . . . 309 | 181/308 (58%) | 1e−98 |
| | Mus musculus (Mouse), 312 aa. | 4 . . . 310 | 224/308 (71%) | |
| Q8NGC7 | Seven transmembrane helix | 26 . . . 310 | 169/287 (58%) | 2e−93 |
| | receptor - Homo sapiens | 39 . . . 325 | 212/287 (72%) | |
| | (Human), 330 aa. | | | |
| CAD57956 | Sequence 107 from Patent | 26 . . . 310 | 169/287 (58%) | 8e−93 |
| | WO0250276 - Homo sapiens | 16 . . . 302 | 211/287 (72%) | |
| | (Human), 307 aa. | | | |
| Q8VFT9 | Olfactory receptor MOR106-2 - | 24 . . . 308 | 168/287 (58%) | 2e−90 |
| | Mus musculus (Mouse), 309 aa. | 21 . . . 307 | 210/287 (72%) | |
| Q8VFT8 | Olfactory receptor MOR106-3 - | 24 . . . 308 | 166/287 (57%) | 4e−90 |
| | Mus musculus (Mouse), 311 aa. | 23 . . . 309 | 212/287 (73%) | |

PFam analysis indicates that the NOV27a protein contains the domains shown in the Table 27F.

TABLE 27F
Domain Analysis of NOV27a

| | | Identities/ | |
|---|---|---|---|
| | NOV27a Match | Similarities for | |
| Pfam Domain | Region | the Matched Region | Expect Value |
| 7tm_1 | 46 . . . 293 | 47/276 (17%) | 4.5e−13 |
| | | 166/276 (60%) | |

### Example 28

The NOV28 clone was analyzed, and the nucleotide and encoded polypeptide sequences are shown in Table 28A.

TABLE 28A
NOV28 Sequence Analysis

| | | |
|---|---|---|
| NOV28a, CG53746-04 | SEQ ID NO: 339 | 1604 bp |
| DNA Sequence | ORF Start: ATG at 648 | ORF Stop: TGA at 1581 |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGTTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28a, CG53746-04 | SEQ ID NO: 340 | 311 aa | MW at 34309.4kD |
| Protein Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28b, GG53746-01 | SEQ ID NO: 341 | 984 bp |
| DNA Sequence | ORF Start: ATG at 24 | ORF Stop: TGA at 957 |

AGTTGGCACCATTCCCAATATAGATGGGGACTGGAAATGACACCACTGTGGTAGAGTTTACTCTTTTG
GGGTTATCTGAGGATACTACAGTTTGTGCTATTTTATTTCTTGTGTTTCTAGGAATTTATGTTGTCAC
CTTAATGGGTAATATCAGCATAATTGTATTGATCAGAAGAAGTCATCATCTTCATACACCCATGTACA
TTTTCCTCTGCCATTTGGCCTTTGTAGACATTGGGTACTCCTCATCAGTCACACCTGTCATGCTCATG
AGCTTCCTAAGGAAAGAAACCTCTCTCCCTGTTGCTGGTTGTGTGGCCCAGCTCTGTTCTGTAGTGAC
GTTTGGTACGGCCGAGTGCTTCCTGCTGGCTGCCATGGCCTATGATCGCTATGTGGCCATCTGCTCAC
CCCTGCTCTACTCTACCTGCATGTCCCCTGGAGTCTGCATCATCTTAGTGGGCATGTCCTACCTGGGT
GGATGTGTGAATGCTTGGACATTCATTGGCTGCTTATTAAGACTGTCCTTCTGTGGGCCAAATAAAGT
CAATCACTTTTTCTGTGACTATTCACCACTTTTGAAGCTTGCTTGTTCCCATGATTTTACTTTTGAAA
TAATTCCAGCTATCTCTTCTGGATCTATCATTGTGGCCACTGTGTGTGTCATAGCCATATCCTACATC
TATATCCTCATCACCATCCTGAAGATGCACTCCACCAAGGGCCGCCACAAGGCCTTCTCCACCTGCAC
CTCCCACCTCACTGCAGTCACTCTGTTCTATGGGACCATTACCTTCATTTATGTGATGCCCAAGTCCA
GCTACTCAACTGACCAGAACAAGGTGGTGTCTGTGTTCTACACCGTGGTGATTCCCATGTTGAACCCC
CTGATCTACAGCCTCAGGAACAAGGAGATTAAGGGGGCTCTGAAGAGAGAGCTTAGAATAAAAATATT
TTCTTGATGAAACTAGTTAGTTTGAAGAATCT

| | | | |
|---|---|---|---|
| NOV28b, CG53746-01 | SEQ ID NO: 342 | 311 aa | MW at 34295.4kD |
| Protein Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPVAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28c, CG53746-02 | SEQ ID NO: 343 | 952 bp |
| DNA Sequence | ORF Start: ATG at 7 | ORF Stop: TGA at 940 |

ATATAGATGGGGACTGGAAATGACACCACTGTGGTAGAGTTTACTCTTTTGGGGTTATCCGAGGATAC
TACAGTTTGTGCTATTTTATTTCTTGTGTTTCTAGGAATTTATGTTGTCACCTTAATGGGTAATATCA
GCATAATTGTATTGATCAGAAGAAGTCATCATCTTCATACACCCATGTACATTTTCCTCTGCCATTTG
GCCTTTGTAGACATTGGGTACTCCTCATCAGTCACACCTGTCATGCTCATGAGCTTCCTAAGGAAAGA
AACCTCTCTCCCTGTTGCTGGTTGTGTGGCCCAGCTCTGTTCTGTGGTGACGTTTGGTACGGCCGAGT
GCTTCCTGCTGGCTGCCATGGCCTATGATCGCTATGTGGCCATCTGCTCACCCCTGCTCTACTCTACC
TGCATGTCCCCTGGAGTCTGCATCATCTTAGTGGGCATGTCCTACCTGGGTGGATGTGTGAATGCTTG
GACATTCATTGGCTGCTTATTAAGACTGTCCTTCTGTGGGCCAAATAAAGTCAATCACTTTTTCTGTG
ACTATTCACCACTTTTGAAGCTTGCTTGTTCCCATGATTTTACTTTTGAAATAATTCCAGCTATCTCT
TCTGGATCTATCATTGTGGCCACTGTGTGTGTCATAGCCATATCCTACATCTATATCCTCATCACCAT
CCTGAAGATGCACTCCACCAAGGGCCGCCACAAGGCCTTCTCCACCTGCACCTCCCACCTCACTGCAG
TCACTCTGTTTTATGGGACCATTACCTTCATTTATGTGATGCCCAAGTCCAGCTACTCAACTGACCAG
AACAAGGTGGTGTCTGTGTTCTACACCGTGGTGATTCCCATGTTGAACCCCCTGATCTACAGCCTCAG
GAACAAGGAGATTAAGGGGGCTCTGAAGAGAGAGCTTAGAATAAAAATATTTTCTTGATGAAACTAGT

| | | | |
|---|---|---|---|
| NOV28c, CG53746-02 | SEQ ID NO: 344 | 311 aa | MW at 34295.4kD |
| Protein Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPVAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28d, GG53746-03 | SEQ ID NO: 345 | 964 bp |
| DNA Sequence | ORF Start: ATG at 8 | ORF Stop: TGA at 941 |

TATATAGATGGGGACTGGAAATGACACCACTGTGGTAGAGTTTACTCTTTTGGGGTTATCCGAGGATA
CTACAGTTTGTGCTATTTTATTTCTTGTGTTTCTAGGAATTTATGTTGTCACCTTAATGGGTAATATC
AGCATAATTGTATTGATCAGAAGAAGTCATCATCTTCATACACCCATGTACATTTTCCTCTGCCATTT
GGCCTTTGTAGACATTGGGTAQTCCTCATCAGTCACACCTGTCATGAGCATGAGCTTCCTAAGGAAAG
AAACCTCTCTCCCTGTTGCTGGTTGTGTGGCCCAGCTCTGTTCTGTGGTGACGTTTGGTACGGCCGAG
TGCTTCCTGCTGGCTGCCATGGCCTATGATCGCTATGTGGCCATCTGCTCACCCCTGCTCTACTCTAC
CTGCATGTCCCCTGGAGTCTGCATCATCTTAGTGGGCATGTCCTACCTGGGTGGATGTGTGAATGCTT
GGACATTCATTGGCTGCTTATTAAGACTGTCCTTCTGTGGGCCAAATAAAGTCAATCACTTTTTCTGT
GACTATTCACCACTTTTGAAGCTTGCTTGTTCCCATGATTTTACTTTTGAAATAATTCCAGCTATCTC
TTCTGGATCTATCATTGTGGCCACTGTGTGTGTCATAGCCATATCCTACATCTATATCCTCATCACCA
TCCTGAAGATGCACTCCACCAAGGGCCGCCACAAGGCCTTCTCCACCTGCACCTCCCACCTCACTGCA
GTCACTCTGTTTTATGGGACCATTACCTTCATTTATGTGATGCCCAAGTCCAGCTACTCAACTGACCA
GAACAAGGTGGTGTCTGTGTTCTACACCGTGGTGATTCCCATGTTGAACCCCCTGATCTACAGCCTCA
GGAACAAGGAGATTAAGGGGGCTCTGAAGAGAGAGCTTAGAATAAAAATATTTTCTTGATGAAACTAG
TTAGTTTGAAGA

| | | | |
|---|---|---|---|
| NOV28d, CG53746-03 | SEQ ID NO: 346 | 311 aa | MW at 34295.4kD |
| Protein Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPVAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28e, SNP13373967 of | SEQ ID NO: 347 | 1604 bp |
| CG53746-04, DNA Sequence | ORF Start: ATG at 648 | ORF Stop: TGA at 1581 |
| | SNP Pos: 466 | SNP Change: T to C |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28e, SNP13373967 of | SEQ ID NO: 348 | 311 aa | MW at 34309.4kD |
| CG53746-04, Protein | | | SNP Change: no change |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28f, SNP13373968 of | SEQ ID NO: 349 | 1604 bp |
| CG53746-04, DNA Sequence | ORF Start: ATG at 648 | ORF Stop: TGA at 1581 |
| | SNP Pos: 550 | SNP Change: T to C |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28f, SNP13373968 of | SEQ ID NO: 350 | 311 aa | MW at 34309.4kD |
| CG53746-04, Protein | | | SNP Change: no change |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28g, SNP13382432 of | SEQ ID NO: 351 | 1604 bp |
| CG53746-04, DNA Sequence | ORF Start: ATG at 648 | ORF Stop: TGA at 1581 |
| | SNP Pos: 644 | SNP Change: T to A |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28g, SNP13382432 of | SEQ ID NO: 352 | 311 aa | MW at 34309.4kD |
| CG53746-04, Protein | | | SNP Change: no change |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28h, SNP13373969 of | SEQ ID NO: 353 | 1604 bp |
| CG53746-04, DNA Sequence | ORF Start: ATG at 648 | ORF Stop: TGA at 1581 |
| | SNP Pos: 695 | SNP Change: A to G |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGGTTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28h, SNP13373969 of | SEQ ID NO: 354 | 311 aa | MW at 34309.4kD |
| CG53746-04, Protein | SNP Pos: 16 | | SNP Change: Gly to Gly |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28i, SNP13373970 of | SEQ ID NO: 355 | 1604 bp |
| CG53746-04, DNA Sequence | ORF Start: ATG at 648 | ORF Stop: TGA at 1581 |
| | SNP Pos: 927 | SNP Change: A to G |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTGTTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28i, SNP13373970 of | SEQ ID NO: 356 | 311 aa | MW at 34295.4kD |
| CG53746-04, Protein | SNP Pos: 94 | | SNP Change: Ile to Val |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPVAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28j, SNP13373971 of | SEQ ID NO: 357 | 1604 bp |
| CG53746-04, DNA Sequence | ORF Start: ATG at 648 | ORF Stop: TGA at 1581 |
| | SNP Pos: 959 | SNP Change: A to G |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTGGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28j, SNP13373971 of | SEQ ID NO: 358 | 311 aa | MW at 34309.4kD |
| CG53746-04, Protein | SNP Pos: 104 | | SNP Change: Val to Val |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28k, SNP13373972 of | SEQ ID NO: 359 | 1604 bp |
| CG53746-04, DNA Sequence | ORF Start: ATG at 648 | ORF Stop: TGA at 1581 |
| | SNP Pos: 1058 | SNP Change: C to T |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCTCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACACCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28k, SNP13373972 of | SEQ ID NO: 360 | 311 aa | MW at 34309.4kD |
| CG53746-04, Protein | SNP Pos: 137 | | SNP Change: Ser to Ser |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYTVVIPMLNPLIYSLRNKEIKGALKRELRIKIFS

| | | |
|---|---|---|
| NOV28l, SNP13382433 of | SEQ ID NO: 361 | 1604 bp |
| CG53746-04, DNA Sequence | ORF Start: ATG at 648 | ORF Stop: TGA at 1581 |
| | SNP Pos: 1482 | SNP Change: A to G |

ACTCACTATAGGGCTCGAGCGGCCGCCCGGGCAGGTTGGATATTTGGTGTACTTTTCCTCAATTGTGA
AATCTCTGGGTGGGGACCACAGCTCAGTGTTGAGTTACTGACCTCTTCTTGGTCCTGTGGATTAGCAT
GCAGCTAATCTGTTTGACCTCTGTTTGGAATTCGGAATTCTTAATGACTACACATCTTTGATACAATA
GATGATACCTCAAACATCCTTTTGAACAGCTGTTCTTTTCCATGAGTCTTGGTCTATTCTGACATTTA
TGTCTCCCTTCAATCACGTCTTTGGCCTTAGAAGATTGAGTTACTGGATTCTTTATATATTCTAGTGG
TCATCTCTGAAATGTGCTCAGAGAGCACCTAAATTAACCATCCAATACGAGTTGAGTGTGTTAAGTTA
AAAAAAAAAAAAAGATTTTTCTGAGTATTCCTGACCTTACATCAGTGAACATTTATGCTTTAAAGTCT
TACATAAGATACTGTGTGTGAAAGCATTTTCTTCCCAAATTTACATGAGTGCCTAAATTGTTATACTT
TTTGGTTAAATAGATATTGGAAAAATAAGTGTGCAATTATAGCATTTAATCCCATTATAATATTCATT
TGTTTTTCTTTCAGTTGGCACCATTCCCAATTTAGATGGGGACTGGAAATGACACCACTGTGGTAGAG
TTTACTCTTTTGGGATTATCCGAGGATACTACAGTTTGTGCTATTTTATTTCTTGTGTTTCTAGGAAT
TTATGTTGTCACCTTAATGGGTAATATCAGCATAATTGTATTGATCAGAAGAAGTCATCATCTTCATA
CACCCATGTACATTTTCCTCTGCCATTTGGCCTTTGTAGACATTGGGTACTCCTCATCAGTCACACCT
GTCATGCTCATGAGCTTCCTAAGGAAAGAAACCTCTCTCCCTATTGCTGGTTGTGTGGCCCAGCTCTG
TTCTGTAGTGACGTTTGGTACGGCCGAGTGCTTCCTGCTGGCTGCCATGGCCTATGATCGCTATGTGG
CCATCTGCTCACCCCTGCTCTACTCTACCTGCATGTCCCCTGGAGTCTGCATCATCTTAGTGGGCATG
TCCTACCTGGGTGGATGTGTGAATGCTTGGACATTCATTGGCTGCTTATTAAGACTGTCCTTCTGTGG
GCCAAATAAAGTCAATCACTTTTTCTGTGACTATTCACCACTTTTGAAGCTTGCTTGTTCCCATGATT
TTACTTTTGAAATAATTCCAGCTATCTCTTCTGGATCTATCATTGTGGCCACTGTGTGTGTCATAGCC
ATATCCTACATCTATATCCTCATCACCATCCTGAAGATGCACTCCACCAAGGGCCGCCACAAGGCCTT
CTCCACCTGCACCTCCCACCTCACTGCAGTCACTCTGTTCTATGGGACCATTACCTTCATTTATGTGA
TGCCCAAGTCCAGCTACTCAACTGACCAGAACAAGGTGGTGTCTGTGTTCTACGCCGTGGTGATTCCC
ATGTTGAACCCCCTGATCTACAGCCTCAGGAACAAGGAGATTAAGGGGGCTCTGAAGAGAGAGCTTAG
AATAAAAATATTTTCTTGATGAAACTAGTTAGTTTGAAGA

| | | | |
|---|---|---|---|
| NOV28l, SNP13382433 of | SEQ ID NO: 362 | 311 aa | MW at 34279.4kD |
| CG53746-04, Protein | SNP Pos: 279 | | SNP Change: Thr to Ala |
| Sequence | | | |

MGTGNDTTVVEFTLLGLSEDTTVCAILFLVFLGIYVVTLMGNISIIVLIRRSHHLHTPMYIFLCHLAF
VDIGYSSSVTPVMLMSFLRKETSLPIAGCVAQLCSVVTFGTAECFLLAAMAYDRYVAICSPLLYSTCM
SPGVCIILVGMSYLGGCVNAWTFIGCLLRLSFCGPNKVNHFFCDYSPLLKLACSHDFTFEIIPAISSG
SIIVATVCVIAISYIYILITILKMHSTKGRHKAFSTCTSHLTAVTLFYGTITFIYVMPKSSYSTDQNK
VVSVFYAVVIPMLNPLIYSLRNKEIKGALKRELRIKIFS

A ClustalW comparison of the above protein sequences yields the following sequence alignment shown in Table 28B.

TABLE 28B
Comparison of the NOV28 protein sequences.

NOV28a MGTGNDTTVVEFTLLGLSEDTTVCAILFLVFLGIYVVTLMGNISIIVLIRRSHHLHTPMY
NOV25b MGTGNDTTVVEFTLLGLSEDTTVCAILFLVFLGIYVVTLMGNISIIVLIRRSHHLHTPMY
NOV25c MGTGNDTTVVEFTLLGLSEDTTVCAILFLVFLGIYVVTLMGNISIIVLIRRSHHLHTPMY
NOV25d MGTGNDTTVVEFTLLGLSEDTTVCAILFLVFLGIYVVTLMGNISIIVLIRRSHHLNTPMY
NOV28a IFLCHLAFVDIGYSSSVTPVMLMSFLRKETSLPIAGCVAQLCSVVTFGTAECFLLAAMAY
NOV28b IFLCHLAFVDIGYSSSVTPVMLMSFLRKETSLPVAGCVAQLCSVVTFGTAECFLLAAMAY
NOV28c IFLCHLAFVDIGYSSSVTPVMLMSFLRKETSLPVAGCVAQLCSVVTFGTAECFLLAAMAY
NOV28d IFLCHLAFVDIGYSSSVTPVMLMSFLRKETSLPVAGCVAQLCSVVTFGTAECFLLAAMAY
NOV28a DRYVAICSPLLYSTCMSPGVCIILVGMSYLGGCVNAWTFIGCLLRLSFCGPNKVNHFFCD
NOV28b DRYVAICSPLLYSTcMSPGVCIILVGMSYLGGCVNAWTFIGCLLRLSFCGPNKVNHFFCD
NOV28c DRYVAICSPLLYSTcMSPGVCIILVGMSYLGGCVNAWTFIGCLLRLSFCGPNKVNHFFCD
NOV28d DRYVAICSPLLYSTCMSPGVCIILVGMSYLGGCVNAWTFIGCLLRLSFCGPNKVNHFFCD
NOV28a YSPLLKLACSHDFTFEIIPAISSGSIIVATVCVIAISYIYILITILKMHSTKGRHKAFST
NOV28b YSPLLKLACSHDFTFEIIPAISSGSIIVATVCVIAISYIYILITILKMHSTKGRHKAFST
NOV28c YSPLLKLACSHDFTFEIIPAISSGSIIVATVCVIAISYIYILITILKMHSTKGRHKAFST
NOV28d YSPLLKLACSHDFTFEIIPAISSGSIIVATVCVIAISYIYILITILKMHSTKGRHKAFST
NOV28a CTSHLTAVTLFYGTITFIYVMPKSSYSTDQNKVVSVFYTVVIPMLNPLIYSLRNKEIKGA
NOV28b CTSHLTAVTLFYGTITFIYVMPKSSYSTDQNKVVSVFYTVVIPMLNPLIYSLRNKEIKGA
NOV28c CTSHLTAVTLFYGTITFIYVMPKSSYSTDQNKVVSVFYTVVIPMLNPLIYSLRNKEIKGA
NOV28d CTSHLTAVTLFYGTITFIYVMPKSSYSTDQNKVVSVFYTVVIPMLNPLIYSLRNKEIKGA
NOV28a LKRELRIKIFS
NOV28b LKRELRIKIFS
NOV28c LKRELRIKIFS
NOV28d LKRELRIKIFS
NOV28a (SEQ ID NO: 340)
NOV28b (SEQ ID NO: 342)
NOV28c (SEQ ID NO: 344)
NOV28d (SEQ ID NO: 346)

Further analysis of the NOV28a protein yielded the following properties shown in Table 28C.

TABLE 28C
Protein Sequence Properties NOV28a

| | |
|---|---|
| SignalP analysis: | Cleavage site between residues 42 and 43 |
| PSORT II analysis: | |

| | |
|---|---|
| PSG: | a new signal peptide prediction method |
| | N-region: length 11; pos.chg 0; neg.chg 2 |
| | H-region: length 7; peak value 0.00 |
| | PSG score: −4.40 |
| GvH: | von Heijne's method for signal seq. recognition |
| | GvH score (threshold: −2.1): −5.11 |
| | possible cleavage site: between 38 and 39 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 6 | | |
| | INTEGRAL | Likelihood = −11.46 | Transmembrane 23-39 |
| | INTEGRAL | Likelihood = −1.49 | Transmembrane 101-117 |
| | INTEGRAL | Likelihood = −2.23 | Transmembrane 140-156 |
| | INTEGRAL | Likelihood = −9.82 | Transmembrane 206-222 |
| | INTEGRAL | Likelihood = −0.85 | Transmembrane 245-261 |
| | INTEGRAL | Likelihood = −3.13 | Transmembrane 273-289 |

| | |
|---|---|
| PERIPHERAL | Likelihood = 1.48 (at 61) |

| | |
|---|---|
| | ALOM score: −11.46 (number of TMSs: 6) |
| MTOP: | Prediction of membrane topology (Hartmann et al.) |
| | Center position for calculation: 30 |
| | Charge difference: 7.5 C(3.5) − N(−4.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 5.92 |
| Hyd Moment(95): | 6.18 | G content: | 2 |
| D/E content: | 2 | S/T content: | 3 |

Score: −7.11

Gavel: prediction of cleavage sites for mitochondrial preseq

cleavage site motif not found

NUCDISC: discrimination of nuclear localization signals

pat4: none
pat7: none
bipartite: none
content of basic residues: 6.8%
NLS Score: −0.47

KDEL: ER retention motif in the C-terminus: none
ER Membrane Retention Signals:

KKXX-like motif in the C-terminus: IKIF

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
22.2%: vacuolar
11.1%: Golgi
11.1%: mitochondrial

>> prediction for CG53746-04 is end (k = 9)

A search of the NOV28a protein against the Geneseq database, a proprietary database that contains sequences published in patents and patent publication, yielded several homologous proteins shown in Table 28D.

TABLE 28D
Geneseq Results for NOV28a

| | | NOV28a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66704 | Human GPCR2c polypeptide - | 1 . . . 311 | 311/311 (100%) | e−180 |
| | Homo sapiens, 311 aa. | 1 . . . 311 | 311/311 (100%) | |
| | [WO200160865-A2, 23-AUG- | | | |
| | 2001] | | | |
| AAU95624 | Human olfactory and pheromone | 1 . . . 311 | 310/311 (99%) | e−180 |
| | G protein-coupled receptor #111 - | 1 . . . 311 | 311/311 (99%) | |
| | Homo sapiens, 311 aa. | | | |
| | [WO200224726-A2, 28-MAR- | | | |
| | 2002] | | | |
| AAG66702 | Human GPCR2a polypeptide - | 1 . . . 311 | 310/311 (99%) | e−180 |
| | Homo sapiens, 311 aa. | 1 . . . 311 | 311/311 (99%) | |
| | [WO200160865-A2, 23-AUG- | | | |
| | 2001] | | | |
| AAE25068 | Human G-protein coupled | 1 . . . 311 | 309/311 (99%) | e−180 |
| | receptor (GCREC)-8 protein - | 1 . . . 311 | 311/311 (99%) | |
| | Homo sapiens, 311 aa. | | | |
| | [WO200246230-A2, 13-JUN- | | | |
| | 2002] | | | |
| AAG66703 | Human GPCR2b polypeptide - | 1 . . . 311 | 309/311 (99%) | e−180 |
| | Homo sapiens, 311 aa. | 1 . . . 311 | 310/311 (99%) | |
| | [WO200160865-A2, 23-AUG- | | | |
| | 2001] | | | |

In a BLAST search of public sequence databases, the NOV28a protein was found to have homology to the proteins shown in the BLASTP data in Table 28E.

TABLE 28E
Public BLASTP Results for NOV28a

| | | NOV28a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8WZ94 | Olfactory receptor 5P3 | 1 . . . 311 | 310/311 (99%) | e−180 |
| | (Olfactory receptor-like protein | 1 . . . 311 | 311/311 (99%) | |
| | JCG1) - Homo sapiens (Human), | | | |
| | 311 aa. | | | |
| CAD37573 | Sequence 175 from Patent | 1 . . . 311 | 304/311 (97%) | e−175 |
| | WO0224726 - Homo sapiens | 1 . . . 311 | 307/311 (97%) | |
| | (Human), 311 aa. | | | |
| Q8NGM2 | Seven transmembrane helix | 59 . . . 311 | 252/253 (99%) | e−145 |
| | receptor - Homo sapiens | 1 . . . 253 | 253/253 (99%) | |
| | (Human), 253 aa. | | | |
| Q8VG42 | Olfactory receptor MOR204-6 - | 1 . . . 303 | 240/303 (79%) | e−138 |
| | Mus musculus (Mouse), 310 aa. | 1 . . . 303 | 267/303 (87%) | |
| Q8VEZ0 | Olfactory receptor MOR204-32 - | 1 . . . 303 | 236/303 (77%) | e−136 |
| | Mus musculus (Mouse), 312 aa. | 1 . . . 303 | 266/303 (86%) | |

PFam analysis indicates that the NOV28a protein contains the domains shown in the Table 28F.

TABLE 28F
Domain Analysis of NOV28a

| | NOV28a | Identities/ | |
|---|---|---|---|
| | Match | Similarities | |
| Pfam Domain | Region | for the Matched Region | Expect Value |
| 7tm_1 | 41 . . . 290 | 56/276 (20%) | 2.5e−34 |
| | | 179/276 (65%) | |

### Example 29

The NOV29 clone was analyzed, and the nucleotide and encoded polypeptide sequences are shown in Table 29A.

TABLE 29A
NOV29 Sequence Analysis

| | | |
|---|---|---|
| NOV29a, CG53767-02 | SEQ ID NO: 363 | 1378 bp |
| DNA Sequence | ORF Start: ATG at 473 | ORF Stop: TAA at 1331 |

TGAAATTGGAGAGTCATACAAAATCCAGAATGGAGAAAAGAGAGATAAACAGTGCTTTTGGGGTAAAA
TCCAGGTATAGGTATTTGGGCCCACTCTATGTCATCTATACATATTATCATATTAGAATCATTCTTCT
CTTCACTCAGGCTCCTGACCTTGACCTCTTTCCAGGTGCATTTAACTCCCTTTCCTCAGGTTCTCTAA
GAGTTGCCTGACACTTGCCCAGGTCCCCTGAATAAAATACAGGTAAATCAGTGCATAAGAAGCTTTAT
ACAGTGATCTCAGACTTGTGACCAAGAGCCCCTCTGCACCATTGATAGACAGTGGCCGTGCCCACCAG
GAGGATGGGCAATCACACTGCAGTGAGCCTATTCCTTCTGTGGGGATTTTCCAGTTTTTCAGACCTGC
AGAGTCTACTTTTTGTGGTGATTCTCTTCTACATGTGACCATCCTAGCTGCAAACGTGTCCATAATGG
GGGCCATCAAGCTCAGCCACAACCTTCACACTCCTATGTACTTTTTCCTCTGTGGCCTGTCCTTTTCA
GAAACTTGTACCACTGTGGTAGTAATCCCTCGCATGTTGGTGGACTTTCTATCAGAGAGCAAGACCAT
TTCTCTTCCTGAGTGTGCCACACAGATGTTTTTCTTTCTGGGCTTTGCATCCAACAACTGTTTCATCA
TGGCCGCTATGTCCTACGACCGCTACACGGCCATCCACAACCCACTGCAGTACCACACCCTTATGACA
AGAAAGATCTGCTTGCAGATGATGATGGCTTCTTGGATGGTTGGGTTCCTGTTTTCTCTGTGCATCAT
CGTCACTGTATTCAACTTGTCTCTTTGCGACTTGAACACTATCCAGCACTATTTCTGTGATATCTCAC
CAGTGGTCTCCCTTGCTTGTAATTACACTTTCTATCATGAAATGGCTATTTTTGTGCTCTCTGCCTTT
GTGTTGGTGGGCAGCTGTATTTTAATTATGATTTCCTATGTCTTCATTGTGTTCATAGTCATAAAGAT
GCCCTCTGCAAAGGGGAGGTCTAAGGCCTTCTCAACTTGCTCCTCCCACCTCACTGTTGTGTCCATAC
ACTATGGATTTGCTTGCTTTGTCTATTTGAGGCCCAAGAACAGCAACTCCTTCGATGAAGACATGCCG
ACGGCCATGATATATACAATACTGATGCCTCTGCTTAACCCCATCGTGTACAGTCTGAGAAACAAAGA
AATGCAGATAGCCCTAAGAAAAACACTAGGCAGTGTATTTGGGGTTTTCCCTCAGAAGACAAAAAAAG
AGCCTGAACATTTAAAAAAATTACACAGCATTGATAAATAAAGGTGAGAAAAGCGAAAAAAAAAAAAA
AAAAAAAAAAAAAAAGGG

| | | | |
|---|---|---|---|
| NOV29a, CG53767-02 | SEQ ID NO: 364 | 286 aa | MW at 32538.4kD |
| Protein Sequence | | | |

MGAIKLSHNLHTPMYFFLCGLSFSETCTTVVVIPRMLVDFLSESKTISLPECATQMFFFLGFASNNCF
IMAANSYDRYTAIHNPLQYHTLMTRKICLQMMMASWMVGFLFSLCIIVTVFNLSLCDLNTIQHYFCDI
SPVVSLACNYTFYHEMAIFVLSAFVLVGSCILIMISYVFIVFIVIKMPSAKGRSKAFSTCSSHLTVVS
IHYGFACFVYLRPKNSNSFDEDMPTAMIYTILMPLLNPIVYSLRNKEMQIALRKTLGSVFGVFPQKTK
KEPEHLKKLHSIDK

| | | |
|---|---|---|
| NOV29b, CG53767-01 | SEQ ID NO: 365 | 1028 bp |
| DNA Sequence | ORF Start: ATG at 23 | ORF Stop: TAA at 1007 |

AGTGGCCGTGCCCACCAGGAGGATGGGCAATCACACTGCAGTGAGCCTATTCCTTCTGTGGGGATTTT
CCAGTTTTTCAGACCTGCAGAGTCTACTTTGTGGTGATTCTCTTCTACATGTGACCATCCTAGCTGCA
AACGTGTCCATAATGGGGGCCATCAAGCTCAGCCACAACCTTCACACTCCTATGTACTTTTTCCTCTG
TGGCCTGTCCTTTTCAGAAACTTGTACCACTGTGGTAGTAATCCCTCGCATGTTGGTGGACTTTCTAT
CAGAGAGCAAGACCATTTCTCTTCCTGAGTGTGCCACACAGATGTTTTTCTTTCTGGGCTTTGCATCC
AACAACTGTTTCATCATGGCCGCTATGTCCTACGACCGCTACACGGCCATCCACAACCCACTGCAGTA
CCACACCCTTATGACAAGAAAGATCTGCTTGCAGATGATGATGGCTTCTTGGATGGTTGGGTTCCTGT
TTTCTCTGTGCATCATCGTCACTGTATTCAACTTGTCTCTTTGCGACTTGAACACTATCCAGCACTAT
TTCTGTGATATCTCACCAGTGGTCTCCCTTGCTTGTAATTACACTTTCTATCATGAAATGGCTATTTT
TGTGCTCTCTGCCTTTGTGTTGGTGGGCAGCTGTATTTTAATTATGATTTCCTATGTCTTCATTGTGT
TCATAGTCATAAAGATGCCCTCTGCAAAGGGGAGGTCTAAGGCCTTCTCAACTTGCTCCTCCCACCTC
ACTGTTGTGTCCATACACTATGGATTTGCTTGCTTTGTCTATTTGAGGCCCAAGAACAGCAACTCCTT
CGATGAAGACATGCTGACGGCCATGATATATACAATACTGATGCCTCTGCTTAACCCCATCGTGTACA
GTCTGAGAAACAAAGAAATGCAGATAGCCCTAAGAAAAACACTAGGCAGTGTATTTGGGGTTTTCCCT
CAGAAGACAAAAAAAGAGCCTGAACATTTAAAAAAATTACACAGCATTGATAAATAAAGGTGAGAAAA
GTGGAGTA

| | | | |
|---|---|---|---|
| NOV29b, CG53767-01 | SEQ ID NO: 366 | 328 aa | MW at 37015.5kD |
| Protein Sequence | | | |

MGNHTAVSLFLLWGFSSFSDLQSLLCGDSLLHVTILAANVSIMGAIKLSHNLHTPMYFFLCGLSFSET
CTTVVVIPRMLVDFLSESKTISLPECATQMFFFLGFASNNCFIMAAMSYDRYTAIHNPLQYHTLMTRK
ICLQMMMASWMVGFLFSLCIIVTVFNLSLCDLNTIQHYFCDISPVVSLACNYTFYHEMAIFVLSAFVL
VGSCILIMISYVFIVFIVIKMPSAKGRSKAFSTCSSHLTVVSIHYGFACFVYLRPKNSNSFDEDMLTA
MIYTILMPLLNPIVYSLRNKEMQIALRKTLGSVFGVFPQKTKKEPEHLKKLHSIDK

| | | |
|---|---|---|
| NOV29c, SNP13382437 of | SEQ ID NO: 367 | 1378 bp |
| CG53767-02, DNA Sequence | ORF Start: ATG at 473 | ORF Stop: TAA at 1331 |
| | SNP Pos: 961 | SNP Change: G to A |

TGAAATTGGAGAGTCATACAAAATCCAGAATGGAGAAAAGAGAGATAAACAGTGCTTTTGGGGTAAAA
TCCAGGTATAGGTATTTGGGCCCACTCTATGTCATCTATACATATTATCATATTAGAATCATTCTTCT
CTTCACTCAGGCTCCTGACCTTGACCTCTTTCCAGGTGCATTTAACTCCCTTTCCTCAGGTTCTCTAA
GAGTTGCCTGACACTTGCCCAGGTCCCCTGAATAAAATACAGGTAAATCAGTGCATAAGAAGCTTTAT
ACAGTGATCTCAGACTTGTGACCAAGAGCCCCTCTGCACCATTGATAGACAGTGGCCGTGCCCACCAG
GAGGATGGGCAATCACACTGCAGTGAGCCTATTCCTTCTGTGGGGATTTTCCAGTTTTTCAGACCTGC
AGAGTCTACTTTTTGTGGTGATTCTCTTCTACATGTGACCATCCTAGCTGCAAACGTGTCCATAATGG
GGGCCATCAAGCTCAGCCACAACCTTCACACTCCTATGTACTTTTTCCTCTGTGGCCTGTCCTTTTCA
GAAACTTGTACCACTGTGGTAGTAATCCCTCGCATGTTGGTGGACTTTCTATCAGAGAGCAAGACCAT
TTCTCTTCCTGAGTGTGCCACACAGATGTTTTTCTTTCTGGGCTTTGCATCCAACAACTGTTTCATCA
TGGCCGCTATGTCCTACGACCGCTACACGGCCATCCACAACCCACTGCAGTACCACACCCTTATGACA
AGAAAGATCTGCTTGCAGATGATGATGGCTTCTTGGATGGTTGGGTTCCTGTTTTCTCTGTGCATCAT
CGTCACTGTATTCAACTTGTCTCTTTGCGACTTGAACACTATCCAGCACTATTTCTGTGATATCTCAC
CAGTGGTCTCCCTTGCTTGTAATTACACTTTCTATCATGAAATGGCTATTTTTGTGCTCTCTGCCTTT
GTGTTGGTAGGCAGCTGTATTTTAATTATGATTTCCTATGTCTTCATTGTGTTCATAGTCATAAAGAT
GCCCTCTGCAAAGGGGAGGTCTAAGGCCTTCTCAACTTGCTCCTCCCACCTCACTGTTGTGTCCATAC
ACTATGGATTTGCTTGCTTTGTCTATTTGAGGCCCAAGAACAGCAACTCCTTCGATGAAGACATGCCG
ACGGCCATGATATATACAATACTGATGCCTCTGCTTAACCCCATCGTGTACAGTCTGAGAAACAAAGA
AATGCAGATAGCCCTAAGAAAAACACTAGGCAGTGTATTTGGGGTTTTCCCTCAGAAGACAAAAAAAG
AGCCTGAACATTTAAAAAAATTACACAGCATTGATAAATAAAGGTGAGAAAGCGAAAAAAAAAAAAAA
AAAAAAAAAAAAAAAGGG

| | | | |
|---|---|---|---|
| NOV29c, SNP13382437 of | SEQ ID NO: 368 | 286 aa | MW at 32538.4kD |
| CG53767-02, Protein | SNP Pos: 163 | | SNP Change: Val to Val |
| Sequence | | | |

MGAIKLSHNLHTPMYFFLCGLSFSETCTTVVVIPRNLVDFLSESKTISLPECATQMFFFLGFASNNCF
IMAANSYDRYTAIHNPLQYHTLMTRKICLQMMMASWMVGFLFSLCIIVTVFNLSLCDLNTIQHYFCDI
SPVVSLACNYTFYHEMAIFVLSAFVLVGSCILIMISYVFIVFIVIKMPSAKGRSKAFSTCSSHLTVVS
IHYGFACFVYLRPKNSNSFDEDMPTAMIYTILMPLLNPIVYSLRNKEMQIALRKTLGSVFGVFPQKTK
KEPEHLKKLHSIDK

| | | |
|---|---|---|
| NOV29d, SNP13382436 of | SEQ ID NO: 369 | 1378 bp |
| CG53767-02, DNA Sequence | ORF Start: ATG at 473 | ORF Stop: TAA at 1331 |
| | SNP Pos: 1283 | SNP Change: A to G |

TGAAATTGGAGAGTCATACAAAATCCAGAATGGAGAAAAGAGAGATAAACAGTGCTTTTGGGGTAAAA
TCCAGGTATAGGTATTTGGGCCCACTCTATGTCATCTATACATATTATCATATTAGAATCATTCTTCT
CTTCACTCAGGCTCCTGACCTTGACCTCTTTCCAGGTGCATTTAACTCCCTTTCCTCAGGTTCTCTAA
GAGTTGCCTGACACTTGCCCAGGTCCCCTGAATAAAATACAGGTAAATCAGTGCATAAGAAGCTTTAT
ACAGTGATCTCAGACTTGTGACCAAGAGCCCCTCTGCACCATTGATAGACAGTGGCCGTGCCCACCAG
GAGGATGGGCAATCACACTGCAGTGAGCCTATTCCTTCTGTGGGGATTTTCCAGTTTTTCAGACCTGC
AGAGTCTACTTTTTGTGGTGATTCTCTTCTACATGTGACCATCCTAGCTGCAAACGTGTCCATAATGG
GGGCCATCAAGCTCAGCCACAACCTTCACACTCCTATGTACTTTTTCCTCTGTGGCCTGTCCTTTTCA
GAAACTTGTACCACTGTGGTAGTAATCCCTCGCATGTTGGTGGACTTTCTATCAGAGAGCAAGACCAT
TTCTCTTCCTGAGTGTGCCACACAGATGTTTTTCTTTCTGGGCTTTGCATCCAACAACTGTTTCATCA
TGGCCGCTATGTCCTACGACCGCTACACGGCCATCCACAACCCACTGCAGTACCACACCCTTATGACA
AGAAAGATCTGCTTGCAGATGATGATGGCTTCTTGGATGGTTGGGTTCCTGTTTTCTCTGTGCATCAT
CGTCACTGTATTCAACTTGTCTCTTTGCGACTTGAACACTATCCAGCACTATTTCTGTGATATCTCAC
CAGTGGTCTCCCTTGCTTGTAATTACACTTTCTATCATGAAATGGCTATTTTTGTGCTCTCTGCCTTT
GTGTTGGTGGGCAGCTGTATTTTAATTATGATTTCCTATGTCTTCATTGTGTTCATAGTCATAAAGAT
GCCCTCTGCAAAGGGGAGGTCTAAGGCCTTCTCAACTTGCTCCTCCCACCTCACTGTTGTGTCCATAC
ACTATGGATTTGCTTGCTTTGTCTATTTGAGGCCCAAGAACAGCAACTCCTTCGATGAAGACATGCCG
ACGGCCATGATATATACAATACTGATGCCTCTGCTTAACCCCATCGTGTACAGTCTGAGAAACAAAGA
AATGCAGATAGCCCTAAGAAAAACACTAGGCAGTGTATTTGGGGTTTTCCCTCAGAAGGCAAAAAAAG
AGCCTGAACATTTAAAAAAATTACACAGCATTGATAAATAAAGGTGAGAAAAGCGAAAAAAAAAAAAA
AAAAAAAAAAAAAAAGGG

| | | | |
|---|---|---|---|
| NOV29d, SNP13382436 of | SEQ ID NO: 370 | 286 aa | MW at 32508.4kD |
| CG53767-02, Protein | SNP Pos: 271 | SNP Change: Thr to Ala | |
| Sequence | | | |

MGAIKLSHNLHTPMYFFLCGLSFSETCTTVVVIPRMLVDFLSESKTISLPECATQMFFFLGFASNNCF
IMAANSYDRYTAIHNPLQYHTLMTRKICLQMMMASWMVGFLFSLCIIVTVFNLSLCDLNTIQHYFCDI
SPVVSLACNYTFYHEMAIFVLSAFVLVGSCILIMISYVFIVFIVIKMPSAKGRSKAFSTCSSHLTVVS
IHYGFACFVYLRPKNSNSFDEDMPTAMIYTILMPLLNPIVYSLRNKEMQIALRKTLGSVFGVFPQKAK
KEPEHLKKLHSIDK

A ClustalW comparison of the above protein sequences yields the following sequence alignment shown in Table 29B.

TABLE 29B
Comparison of the NOV29 protein sequences.

NOV29a ------------------------------------------MGAIKLSHNLHTPMYFFL
NOV29b MGNHTAVSLFLLWGFSSFSDLQSLLCGDSLLHVTILAANVSIMGAIKLSHNLHTPMYFFL
NOV29a CGLSFSETCTTVVVIPRNLVDFLSESKTISLPECATQMFFFLGFASNNCFIMAAMSYDRY
NOV29b CGLSFSETCTTVVVIPRMLVDFLSESKTISLPECATQMFFFLGFASNNCFIMAAMSYDRY
NOV29a TAIHNPLQYHTLMTRKICLQMMMASWMVGFLFSLCIIVTVFNLSLCDLNTIQHYFCDISP
NOV29b TAIHNPLQYHTLMTRKICLQMMMASWMVGFLFSLCIIVTVFNLSLCDLNTIQHYFCDISP
NOV29a VVSLACNYTFYHEMAIFVLSAFVLVGSCILIMISYVFIVFIVIKMPSAKGRSKAFSTCSS
NOV29b VVSLACNYTFYMEMAIFVLSAFVLVGSCILIMISYVFIVFIVIKMPSAKGRSKAFSTCSS
NOV29a HLTVVSIHYGFACFVYLRPKNSNSFDEDMPTAMIYTILMPLLNPIVYSLRNKEMQIALRK
NOV29b HLTVVSIHYGFACFVYLRPKNSNSFDEDMLTAMIYTILMPLLNPIVYSLRNKENQIALRK
NOV29a TLGSVFGVFPQKTKKEPEHLKKLHSIDK
NOV29b TLGSVFGVFPQKTKKEPEHLKKLHSIDK
NOV29a (SEQ ID NO: 364)
NOV29b (SEQ ID NO: 366)

Further analysis of the NOV29a protein yielded the following properties shown in Table 29C.

TABLE 29C
Protein Sequence Properties NOV29a

| | |
|---|---|
| SignalP analysis: | Cleavage site between residues 25 and 26 |

PSORT II analysis:
PSG: a new signal peptide prediction method

N-region: length 5; pos.chg 1; neg.chg 0
H-region: length 19; peak value 10.63
PSG score: 6.23

GvH: von Heijne's method for signal seq. recognition

GvH score (threshold: −2.1): −2.51
possible cleavage site: between 29 and 30

>>> Seems to have no N-terminal signal peptide
ALOM: Klein et al's method for TM region allocation

| Init position for calculation: 1 | | |
|---|---|---|
| Tentative number of TMS(s) for the threshold 0.5: 4 | | |
| INTEGRAL | Likelihood = −0.43 | Transmembrane 17-33 |
| INTEGRAL | Likelihood = −7.17 | Transmembrane 105-121 |
| INTEGRAL | Likelihood = −1.33 | Transmembrane 148-164 |
| INTEGRAL | Likelihood = −12.52 | Transmembrane 165-181 |

| | |
|---|---|
| PERIPHERAL | Likelihood = 0.53 (at 231) |

ALOM score: −12.52 (number of TMSs: 4)

MTOP: Prediction of membrane topology (Hartmann et al.)

Center position for calculation: 24
Charge difference: −0.5 C(−1.0) − N(−0.5)
N >= C: N-terminal side will be inside

>>> membrane topology: type 3a
MITDISC: discrimination of mitochondrial targeting seq

| | | | |
|---|---|---|---|
| R content: | 0 | Hyd Moment(75): | 4.10 |
| Hyd Moment(95): | 4.80 | G content: | 2 |
| D/E content: | 1 | S/T content: | 4 |
| Score: −5.17 | | | |

Gavel: prediction of cleavage sites for mitochondrial preseq

R-2 motif at 45 PRM|LV

NUCDISC: discrimination of nuclear localization signals

pat4: none
pat7: PQKTKKE (4) at 268
bipartite: RKTLGSVFGVFPQKTKK at 257
content of basic residues: 7.7%
NLS Score: 0.36

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

55.6%: endoplasmic reticulum
44.4%: mitochondrial

>> prediction for CG53767-02 is end (k = 9)

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP72224 | Human G-protein coupled | 1 . . . 286 | 285/286 (99%) | e−166 |
| | receptor GCREC-18 (olfactory | 44 . . . 329 | 285/286 (99%) | |
| | receptor) - Homo sapiens, 329 aa. | | | |
| | [WO2003000859-A2, 03-JAN- | | | |
| | 2003] | | | |
| ABP95890 | Human GPCR polypeptide SEQ | 1 . . . 286 | 285/286 (99%) | e−166 |
| | ID NO 590 - Homo sapiens, 286 | 1 . . . 286 | 285/286 (99%) | |
| | aa. [WO200216548-A2, 28-FEB- | | | |
| | 2002] | | | |
| AAG66705 | Human GPCR3 polypeptide - | 1 . . . 286 | 285/286 (99%) | e−166 |
| | Homo sapiens, 328 aa. | 43 . . . 328 | 285/286 (99%) | |
| | [WO200160865-A2, 23-AUG- | | | |
| | 2001] | | | |
| AAG71567 | Human olfactory receptor | 1 . . . 271 | 270/271 (99%) | e−156 |
| | polypeptide, SEQ ID NO: 1248 - | 46 . . . 316 | 270/271 (99%) | |
| | Homo sapiens, 316 aa. | | | |
| | [WO200127158-A2, 19-APR- | | | |
| | 2001] | | | |
| ABG59965 | Human DITHP polypeptide #23 - | 1 . . . 258 | 257/258 (99%) | e−149 |
| | Homo sapiens, 328 aa. | 71 . . . 328 | 257/258 (99%) | |
| | [WO200220754-A2, 14-MAR- | | | |
| | 2002] | | | |

In a BLAST search of public sequence databases, the NOV29a protein was found to have homology to the proteins shown in the BLASTP data in Table 29E.

TABLE 29E
Public BLASTP Results for NOV29a

| | | NOV29a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NGM4 | Seven transmembrane helix | 1 . . . 286 | 285/286 (99%) | e−165 |
| | receptor - Homo sapiens | 54 . . . 339 | 285/286 (99%) | |
| | (Human), 339 aa. | | | |
| Q8VF20 | Olfactory receptor MOR267-14 - | 1 . . . 278 | 187/278 (67%) | e−109 |
| | Mus musculus (Mouse), 321 | 44 . . . 321 | 226/278 (81%) | |
| | aa. | | | |
| CAD42418 | Sequence 75 from Patent | 1 . . . 261 | 136/261 (52%) | 3e−75 |
| | WO0212343 - Homo sapiens | 46 . . . 306 | 182/261 (69%) | |
| | (Human), 314 aa. | | | |
| CAD42393 | Sequence 23 from Patent | 1 . . . 261 | 134/261 (51%) | 1e−73 |
| | WO0212343 - Homo sapiens | 44 . . . 304 | 177/261 (67%) | |
| | (Human), 313 aa. | | | |
| Q8NGX3 | Seven transmembrane helix | 1 . . . 261 | 132/261 (50%) | 4e−73 |
| | receptor - Homo sapiens | 47 . . . 306 | 186/261 (70%) | |
| | (Human), 314 aa. | | | |

PFam analysis indicates that the NOV29a protein contains the domains shown in the Table 29F.

TABLE 29F
Domain Analysis of NOV29a

| | NOV29a | Identities/ | |
|---|---|---|---|
| | Match | Similarities | |
| Pfam Domain | Region | for the Matched Region | Expect Value |
| 7tm_1 | 3 . . . 245 | 47/278 (17%) | 3.1e−19 |
| | | 169/278 (61%) | |

### Example 30

The NOV30 clone was analyzed, and the nucleotide and encoded polypeptide sequences are shown in Table 30A.

TABLE 30A
NOV30 Sequence Analysis

| | | |
|---|---|---|
| NOV30a, CG53776-02 | SEQ ID NO: 371 | 1072 bp |
| DNA Sequence | ORF Start: ATG at 12 | ORF Stop: TAG at 1047 |

TTAACATTTTAATGTGCTGTTCTCATTTGGGTTCATTTAGTCAGCAGCTACTTCGTCTCATGAATTCC
CTGAAGGACGGGAATCACACCGCTCTGACGGGGTTCATCCTATTGGGCTTAACAGATGATCCAATCCT
TCGAGTCATCCTCTTCATGATCATCCTATGCATCTACCTGGTAACCATATCTGGTAATCTCAGCATAA
TTATTCTTATCAGAATTTCTTCTCAGCTCCATCATCCTATGTATTTCTTTCTGAGCCACTTGGCTTTT
GCTGACATGGCCTATTCATCTTCTGTCACACCCAACATGCTTGTAAACTTCCTGGTGGAGAGAAATAC
AGTCTCCTACCTTGGATGTGCCATCCAGCTTGGTTCAGCGGCTTTCTTTGCAACAGTCGAATGCGTCT
TTCTGGCTGCCATGGCCTATGACCGCTTTGTGGCAATTTGCAGTCCACTGCTTTATTCAACCAAAATG
TCCACACAAGTCAGTGTCCAGCTACTCTTAGTAGTTTACATAGCTGGTTTTCTCATTGCTGTCTCCTA
TACTACTTCCTTCTATTTTTTACTCTTCTGTGGACCAAATCAAGTCAATCATTTTTTCTGTGATTTCG
CTCCCTTACTTGAACTCTCCTGTTCTGATATCAGTGTCTCCACAGTTGTTCTCTCATTTTCTTCTGGA
TCCATCATTGTGGTCACTGTGTGTGTCATAGCCGTCTGCTACATCTATATCCTCATCACCATCCTGAA
GATGCGCTCCACTGAGGGGCACCACAAGGCCTTCTCCACCTGCACTTCCCACCTCACTGTGGTTACCC
TGTTCTATGGGACCATTACCTTCATTTATGTGATGCCCAATTTTAGCTACTCAACTGACCAGAACAAG
GTGGTGTCTGTGTTGTACACAGTGGTGATTCCCATGTTGAACCCCTTGATCTACAGCCTCAGGAACAA
GGAGATTAAGGGGGCTCTGAAGAGAGAGCTTGTTAGAAAAATACTTTCTCATGATGCTTGTTATTTTA
GTAGAACTTCAAATAATGATATTACATAGAACCCTATCTCTTCTCTTGAGAA

| | | | |
|---|---|---|---|
| NOV30a, CG53776-02 | SEQ ID NO: 372 | 345 aa | MW at 38430.9kD |
| Protein Sequence | | | |

MCCSHLGSFSQQLLRLMNSLKDGNHTALTGFILLGLTDDPILRVILFMIILCIYLVTISGNLSIIILI
RISSQLHHPMYFFLSHLAFADMAYSSSVTPNMLVNFLVERNTVSYLGCAIQLGSAAFFATVECVFLAA
MAYDRFVAICSPLLYSTKMSTQVSVQLLLVVYIAGFLIAVSYTTSFYFLLFCGPNQVNHFFCDFAPLL
ELSCSDISVSTVVLSFSSGSIIVVTVCVIAVCYIYILITILKMRSTEGHHKAFSTCTSHLTVVTLFYG
TITFIYVMPNFSYSTDQNKVVSVLYTVVIPMLNPLIYSLRNKEIKGALKRELVRKILSHDACYFSRTS
NNDIT

| | | |
|---|---|---|
| NOV30b, CG53776-03 | SEQ ID NO: 373 | 1050 bp |
| DNA Sequence | ORF Start: at 207 | ORF Stop: end of sequence |

TTCTCAAGAGAAGAGATAGGGTTCTATGTAATATCATTATTTGAAGTTCTACTAAAATAACAAGCATC
ATGAGAAAGTATTTTTCTAACAAGCTCTCTCTTCAGAGCCCCCTTAATCTCCTTGTTCCTGAGGCTGT
AGATCAGGGGGTTCAACATGGGAATCACCACTGTGTACAACACAGACACCACCTTGTTCTGGTCAGTT
GAGTAGCTAAAATTGGGCATCACATAAATGAAGGTAATGGTCCCATAGAACAGGGTAACCACAGTGAG
GTGGGAAGTGCAGGTGGAGAAGGCCTTGTGGTGCCCCTCAGTGGAGCGCATCTTCAGGATGGTGATGA
GGATATAGATGTAGCAGACGGCTATGACACACACAGTGACCACAATGATGGATCCAGAAGAAAATGAG
AGAACAACTGTGGAGACACTGATATCAGAACAGGAGAGTTCAAGTAAGGGAGCGAAATCACAGAAAAA
ATGATTGACTTGATTTGGTCCACAGAAGAGTAAAAATAGAAGGAAGTAGTATAGGAGACAGCAATGAG
AAAACCAGCTATGTAAACTACTAAGAGTAGCTGGACACTGACTTGTGTGGACATTTTGGTTGAATAAA
GCAGTGGACTGCAAATTGCCACAAAGCGGTCATAGGCCATGGCAGCCAGAAGGACGCATTCGACTGTT
GCAAAGAAAGCCGCTGAACCAAGCTGGATGGCACATCCAAGGTAGGAGACTGTATTTCTCTCCACCAG
GAAGTTTACAAGCATGTTGGGTGTGACAGAAGATGAATAGGCCATGTCAGCAAAAGCCAAGTGGCTCA
GAAAGAAATACATAGGATGATGGAGCTGAGAAGAAATTCTGATAAGAATAATTATGCTGAGATTACCA
GATAGGATGATCATGAAGAGGATGACTCGAAGGATTGGATCATCTGTTAAGCCCAATAGGATGAACCC
CGTCAGAGCGGTGTGATTCCCGTCCTTCAGGGAATTCATGAGACGAAGTAGCTGCTGACTAAATGAAC
CCTAATGAGAACAGCACATTAAAATGTTAA

| | | | |
|---|---|---|---|
| NOV30b, CG53776-03 | SEQ ID NO: 374 | 338 aa | MW at 37640.9kD |
| Protein Sequence | | | |

MCCSHLGSFSQQLLRLMNSLKDGNHTALTGFILLGLTDDPILRVILFMIILCGNLSIIILIRISSQLH
HPMYFFLSHLAFADMAYSSSVTPNMLVNFLVERNTVSYLGCAIQLGSAAFFATVECVFLAAMAYDRFV
AICSPLLYSTKMSTQVSVQLLLVVYIAGFLIAVSYTTSFYFLLFCGPNQVNHFFCDFAPLLELSCSDI
SVSTVVLSFSSGSIIVVTVCVIAVCYIYILITILKMRSTEGHHKAFSTCTSHLTVVTLFYGTITFIYV
MPNFSYSTDQNKVVSVLYTVVIPMLNPLIYSLRNKEIKGALKRELVRKILSHDACYFSRTSNNDIT

| | | |
|---|---|---|
| NOV30c, CG53776-01 | SEQ ID NO: 375 | 1070 bp |
| DNA Sequence | ORF Start: ATG at 27 | ORF Stop: TAG at 1041 |

GAAGCCATCAAATTTATAACATTTTAATGTGCTGTTCTCATTTGGGTTCATTTAGTCAGCAGCTACTT
CGTCTCATGAATTCCCTGAAGGACGGGAATCACACCGCTCTGACGGGGTTCATCCTATTGGGCTTAAC
AGATGATCCAATCCTTCGAGTCATCCTCTTCATGATCATCCTATCTGGTAATCTCAGCATAATTATTC
TTATCAGAATTTCTTCTCAGCTCCATCATCCTATGTATTTCTTTCTGAGCCACTTGGCTTTTGCTGAC
ATGGCCTATTCATCTTCTGTCACACCCAACATGCTTGTAAACTTCCTGGTGGAGAGAAATACAGTCTC
CTACCTTGGATGTGCCATCCAGCTTGGTTCAGCGGCTTTCTTTGCAACAGTCGAATGCGTCCTTCTGG
CTGCCATGGCCTATGACCGCTTTGTGGCAATTTGCAGTCCACTGCTTTATTCAACCAAAATGTCCACA
CAAGTCAGTGTCCAGCTACTCTTAGTAGTTTACATAGCTGGTTTTCTCATTGCTGTCTCCTATACTAC
TTCCTTCTATTTTTTACTCTTCTGTGGACCAAATCAAGTCAATCATTTTTTCTGTGATTTCGCTCCCT
TACTTGAACTCTCCTGTTCTGATATCAGTGTCTCCACAGTTGTTCTCTCATTTTCTTCTGGATCCATC
ATTGTGGTCACTGTGTGTGTCATAGCCGTCTGCTACATCTATATCCTCATCACCATCCTGAAGATGCG
CTCCACTGAGGGGCACCACAAGGCCTTCTCCACCTGCACTTCCCACCTCACTGTGGTTACCCTGTTCT
ATGGGACCATTACCTTCATTTATGTGATGCCCAATTTTAGCTACTCAACTGACCAGAACAAGGTGGTG
TCTGTGTTGTACACAGTGGTGATTCCCATGTTGAACCCCCTGATCTACAGCCTCAGGAACAAGGAGAT
TAAGGGGGCTCTGAAGAGAGAGCTTGTTAGAAAAATACTTTCTCATGATGCTTGTTATTTTAGTAGAA
CTTCAAATAATGATATTACATAGAACCCTATCTCTTCTCTTGAGAATACT

| | | | |
|---|---|---|---|
| NOV30c, CG53776-01 | SEQ ID NO: 376 | 338 aa | MW at 37590.8kD |
| Protein Sequence | | | |

MCCSHLGSFSQQLLRLMNSLKDGNHTALTGFILLGLTDDPILRVILFMIILSGNLSIIILIRISSQLH
HPMYFFLSHLAFADMAYSSSVTPNMLVNFLVERNTVSYLGCAIQLGSAAFFATVECVLLAAMAYDRFV
AICSPLLYSTKMSTQVSVQLLLVVYIAGFLIAVSYTTSFYFLLFCGPNQVNHFFCDFAPLLELSCSDI
SVSTVVLSFSSGSIIVVTVCVIAVCYIYILITILKMRSTEGHHKAFSTCTSHLTVVTLFYGTITFIYV
MPNFSYSTDQNKVVSVLYTVVIPMLNPLIYSLRNKEIKGALKRELVRKILSHDACYFSRTSNNDIT

A ClustalW comparison of the above protein sequences yields the following sequence alignment shown in Table 30B.

TABLE 30B
Comparison of the NOV30 protein sequences.

NOV30a MCCSHLGSFSQQLLRLNNSLKDGNHTALTGFILLGLTDDPILRVILFMIILCIYLVTISG
NOV30b MCCSHLGSFSQQLLRLMNSLKDGNHTALTGFILLGLTDDPILRVILFMIILC-------G
NOV30c MCCSHLGSFSQQLLRLMNSLKDGNHTALTGFILLGLTDDPILRVILFMIILS-------G
NOV30a NLSIIILIRISSQLHHPMYFFLSHLAFADMAYSSSVTPNMLVNFLVERNTVSYLGCAIQL
NOV30b NLSIIILIRISSQLHHPMYFFLSHLAFADMAYSSSVTPNMLVNFLVERNTVSYLGCAIQL
NOV30c NLSIIILIRISSQLHHPMYFFLSHLAFADMAYSSSVTPNMLVNFLVERNTVSYLGCAIQL
NOV30a GSAAFFATVECVFLAAMAYDRFVAICSPLLYSTKMSTQVSVQLLLVVYIAGFLIAVSYTT
NOV30b GSAAFFATVECVFLAAMAYDRFVAICSPLLYSTKMSTQVSVQLLLVVYIAGFLIAVSYTT
NOV30c GSAAFFATVECVLLAAMAYDRFVAICSPLLYSTKMSTQVSVQLLLVVYIAGFLIAVSYTT
NOV30a SFYFLLFCGPNQVNHFFCDFAPLLELSCSDISVSTVVLSFSSGSIIVVTVCVIAVCYIYI
NOV30b SFYFLLFCGPNQVNHFFCDFAPLLELSCSDISVSTVVLSFSSGSIIVVTVCVIAVCYIYI
NOV30c SFYFLLFCGPNQVNHFFCDFAPLLELSCSDISVSTVVLSFSSGSIIVVTVCVIAVCYIYI
NOV30a LITILKMRSTEGHHKAFSTCTSHLTVVTLFYGTITFIYVMPNFSYSTDQNKVVSVLYTVV
NOV30b LITILKMRSTEGHHKAFSTCTSHLTVVTLFYGTITFIYVMPNFSYSTDQNKVVSVLYTVV
NOV30c LITILKMRSTEGHHKAFSTCTSHLTVVTLFYGTITFIYVMPNFSYSTDQNKVVSVLYTVV
NOV30a IPMLNPLIYSLRNKEIKGALKRELVRKILSHDACYFSRTSNNDIT
NOV30b IPMLNPLIYSLRNKEIKGALKRELVRKILSHDACYFSRTSNNDIT
NOV30c IPMLNPLIYSLRNKEIKGALKRELVRKILSHDACYFSRTSNNDIT
NOV30a (SEQ ID NO: 372)
NOV30b (SEQ ID NO: 374)
NOV30c (SEQ ID NO: 376)

Further analysis of the NOV30a protein yielded the following properties shown in Table 30C.

TABLE 30C
Protein Sequence Properties NOV30a

| | |
|---|---|
| SignalP analysis: | Cleavage site between residues 61 and 62 |
| PSORT II analysis: | |

PSG: a new signal peptide prediction method

N-region: length 0; pos.chg 0; neg.chg 0
H-region: length 14; peak value 5.07
PSG score: 0.67

GvH: von Heijne's method for signal seq. recognition

GvH score (threshold: −2.1): −5.40
possible cleavage site: between 57 and 58

>>> Seems to have no N-terminal signal peptide
ALOM: Klein et al's method for TM region allocation

| Init position for calculation: 1 | | |
|---|---|---|
| Tentative number of TMS(s) for the threshold 0.5: 6 | | |
| INTEGRAL | Likelihood = −10.56 | Transmembrane 41-57 |
| INTEGRAL | Likelihood = −2.07 | Transmembrane 122-138 |
| INTEGRAL | Likelihood = −7.70 | Transmembrane 159-175 |
| INTEGRAL | Likelihood = −12.68 | Transmembrane 225-241 |
| INTEGRAL | Likelihood = −2.13 | Transmembrane 264-280 |
| INTEGRAL | Likelihood = −3.66 | Transmembrane 292-308 |

| | |
|---|---|
| PERIPHERAL | Likelihood = 1.91 (at 58) |

ALOM score: −12.68 (number of TMSs: 6)

MTOP: Prediction of membrane topology (Hartmann et al.)

Center position for calculation: 48
Charge difference: 3.0 C(2.0) − N(−1.0)
C > N: C-terminal side will be inside

>>>Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75): | 3.87 |
| Hyd Moment(95): | 1.84 | G content: | 1 |
| D/E content: | 1 | S/T content: | 4 |
| Score: −4.16 | | | |

Gavel: prediction of cleavage sites for mitochondrial preseq

R-2 motif at 25 LRL|MN

NUCDISC: discrimination of nuclear localization signals

pat4: none
pat7: none
bipartite: none
content of basic residues: 5.5%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

44.4%: endoplasmic reticulum
22.2%: vacuolar
11.1%: Golgi
11.1%: vesicles of secretory system
11.1%: mitochondrial

>> prediction for CG53776-02 is end (k = 9)

A search of the NOV30a protein against the Geneseq database, a proprietary database that contains sequences published in patents and patent publication, yielded several homologous proteins shown in Table 30D.

TABLE 30D
Geneseq Results for NOV30a

| | | NOV30a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66706 | Human GPCR4 polypeptide - | 1 . . . 345 | 337/345 (97%) | 0.0 |
| | Homo sapiens, 338 aa. | 1 . . . 338 | 337/345 (97%) | |
| | [WO200160865-A2, 23-AUG- | | | |
| | 2001] | | | |
| ABU11199 | Human G-protein coupled | 17 . . . 345 | 321/329 (97%) | e−180 |
| | receptor GCREC-52 - Homo | 1 . . . 322 | 321/329 (97%) | |
| | sapiens, 322 aa. [WO200279448- | | | |
| | A2, 10-OCT-2002] | | | |
| AAU85166 | G-coupled olfactory receptor #27 - | 17 . . . 345 | 321/329 (97%) | e−180 |
| | Homo sapiens, 322 aa. | 1 . . . 322 | 321/329 (97%) | |
| | [WO200198526-A2, 27-DEC- | | | |
| | 2001] | | | |
| AAU95576 | Human olfactory and pheromone | 17 . . . 345 | 321/329 (97%) | e−180 |
| | G protein-coupled receptor #63 - | 1 . . . 322 | 321/329 (97%) | |
| | Homo sapiens, 322 aa. | | | |
| | [WO200224726-A2, 28-MAR- | | | |
| | 2002] | | | |
| ABP95758 | Human GPCR polypeptide SEQ | 17 . . . 345 | 321/329 (97%) | e−180 |
| | ID NO 326 - Homo sapiens, 322 | 1 . . . 322 | 321/329 (97%) | |
| | aa. [WO200216548-A2, 28-FEB- | | | |
| | 2002] | | | |

In a BLAST search of public sequence databases, the NOV30a protein was found to have homology to the proteins shown in the BLASTP data in Table 30E.

TABLE 30E
Public BLASTP Results for NOV30a

| | | NOV30a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8WZ92 | Olfactory receptor 5P2 | 17 . . . 345 | 321/329 (97%) | e−179 |
| | (Olfactory receptor-like protein | 1 . . . 322 | 321/329 (97%) | |
| | JCG3) - Homo sapiens (Human), | | | |
| | 322 aa. | | | |
| CAD37572 | Sequence 173 from Patent | 17 . . . 345 | 320/329 (97%) | e−178 |
| | WO0224726 - Homo sapiens | 1 . . . 322 | 320/329 (97%) | |
| | (Human), 322 aa. | | | |
| Q8VG09 | Olfactory receptor MOR204-8 - | 17 . . . 330 | 253/314 (80%) | e−145 |
| | Mus musculus (Mouse), 314 aa. | 1 . . . 314 | 279/314 (88%) | |
| Q8VG04 | Olfactory receptor MOR204-13 - | 21 . . . 330 | 246/310 (79%) | e−141 |
| | Mus musculus (Mouse), 314 aa. | 5 . . . 314 | 272/310 (87%) | |
| Q8VFD3 | Olfactory receptor MOR204-16 - | 17 . . . 336 | 239/320 (74%) | e−137 |
| | Mus musculus (Mouse), 321 aa. | 1 . . . 320 | 271/320 (84%) | |

PFam analysis indicates that the NOV30a protein contains the domains shown in the Table 30F.

TABLE 30F
Domain Analysis of NOV30a

| | NOV30a | Identities/ | |
|---|---|---|---|
| | Match | Similarities | |
| Pfam Domain | Region | for the Matched Region | Expect Value |
| DUF40 | 58 . . . 299 | 43/277 (16%) | 0.22 |
| | | 152/277 (55%) | |
| 7tm_1 | 60 . . . 309 | 54/276 (20%) | 9.4e−32 |
| | | 185/276 (67%) | |

### Example 31

The NOV31 clone was analyzed, and the nucleotide and encoded polypeptide sequences are shown in Table 31A.

TABLE 31A
NOV31 Sequence Analysis

| | | |
|---|---|---|
| NOV31a, GG53803-02 | SEQ ID NO: 377 | 1049 bp |
| DNA Sequence | ORF Start: ATG at 54 | ORF Stop: TGA at 996 |

ATATTTTGCTTTGGCAGGAACAATTCTCTTCAACCCTTCCATTAAAAGGAATTATGATGATGGTTTTA
AGGAATCTGAGCATGGAGCCCACCTTTGCCCTTTTAGGTTTCACAGATTACCCAAAGCTTCAGATTCC
TCTCTTCCTTGTGTTTCTGCTCATGTATGTTATCACAGTGGTAGGAAACCTTGGGATGATCATAATAA
TCAAGATTAACCCCAAATTTCACACTCCTATGTACTTTTTCCTTAGTCACCTCTCTTTTGTTGATTTT
TGTTACTCTTCCATTGTCACTCCCAAGCTGCTTGAGAACTTGGTAATGGCAGATAAAAGCATCTTCTA
CTTTAGCTGCATGATGCAGTACTTCCTGTCCTGCACTGCTGTGGTGACAGAGTCTTTCTTGCTGGCAG
TGATGGCCTATGACCGCTTTGTGGCCATCTGCAATCCTCTGCTTTATACAGTGGCCATGTCACAGAGG
CTCTGTGCCCTGCTGGTGGCTGGGTCATATCTCTGGGGCATGTTTGGCCCCTTGGTACTCCTTTGTTA
TGCTCTCCGGTTAAACTTCTCTGGACCTAATGTAATCAACCACTTCTTTTGTGAGTATACTGCTCTCA
TCTCTGTGTCTGGCTCTGATATACTCATCCCCCACCTGCTGCTTTTCAGCTTCGCCACCTTCAATGAG
ATGTGTACACTACTGATCATCCTCACTTCCTATGTTTTCATTTTTGTGACTGTACTAAAAATCCGTTC
TGTTAGTGGGCGCCACAAAGCCTTCTCCACCTGGGCCTCCCACCTGACTGCTATCACCATCTTCCATG
GGACCATCCTTTTCCTTTACTGTGTACCCAACTCCAAAAACTCTCGGCAAACAGTCAAAGTGGCCTCT
GTATTTTACACAGTTGTCAACCCCATGCTGAACCCTCCGATCTACAGCCTAAGGAATAAAGACGTGAA
GGATGCTTTCTGGAAGTTAATACATACACAAGTTCCATTTCACTGAACCAGTCTCAAAAGTTGTTTTC
AATCCAAATGAACAACCCANNNNNNNNNN

| | | | |
|---|---|---|---|
| NOV31a, CG53803-02 | SEQ ID NO: 378 | 314 aa | MW at 35790.4kD |
| Protein Sequence | | | |

MMMVLRNLSMEPTFALLGFTDYPKLQIPLFLVFLLMYVITVVGNLGMIIIIKINPKFHTPMYFFLSHL
SFVDFCYSSIVTPKLLENLVMADKSIFYFSCMMQYFLSCTAVVTESFLLAVMAYDRFVAICNPLLYTV
AMSQRLCALLVAGSYLWGMFGPLVLLCYALRLNFSGPNVINHFFCEYTALISVSGSDILIPHLLLFSF
ATFNEMCTLLIILTSYVFIFVTVLKIRSVSGRHKAFSTWASHLTAITIFHGTILFLYCVPNSKNSRQT
VKVASVFYTVVNPMLNPPIYSLRNKDVKDAFWKLIHTQVPFH

| | | |
|---|---|---|
| NOV31b,GG53803-01 | SEQ ID NO: 379 | 1039 bp |
| DNA Sequence | ORF Start: ATG at 54 | ORF Stop: TGA at 996 |

ATATTTTGCTTTGGCAGGAACAATTCTCTTCAACCCTTCCATTAAAAGGAATTATGATGATGGTTTTA
AGGAATCTGAGCATGGAGCCCACCTTTGCCCTTTTAGGTTTCACAGATTACCCAAAGCTTCAGATTCC
TCTCTTCCTTGTGTTTCTGCTCATGTATGTTATCACAGTGGTAGGAAACCTTGGGATGATCATAATAA
TCAAGATTAACCCCAAATTTCACACTCCTATGTACTTTTTCCTTAGTCACCTCTCTTTTGTTGATTTT
TGTTACTCTTCCATTGTCACTCCCAAGCTGCTTGAGAACTTGGTAATGGCAGATAAAAGCATCTTCTA
CTTTAGCTGCATGATGCAGTACTTCCTGTCCTGCACTGCTGTGGTGACAGAGTCTTTCTTGCTGGCAG
TGATGGCCTATGACCGCTTTGTGGCCATCTGCAATCCTCTGCTTTATACAGTGGCCATGTCACAGAGG
CTCTGTGCCCTGCTGGTGGCTGGGTCATATCTCTGGGGCATGTTTGGCCCCTTGGTACTCCTTTGTTA
TGCTCTCCGGTTAAACTTCTCTGGACCTAATGTAATCAACCACTTCTTTTGTGAGTATACTGCTCTCA
TCTCTGTGTCTGGCTCTGATATACTCATCCCCCACCTGCTGCTTTTCAGCTTCGCCACCTTCAATGAG
ATGTGTACACTACTGATCATCCTCACTTCCTATGTTTTCATTTTTGTGACTGTACTAAAAATCCGTTC
TGTTAGTGGGCGCCACAAAGCCTTCTCCACCTGGGCCTCCCACCTGACTGCTATCACCATCTTCCATG
GGACCATCCTTTTCCTTTACTGTGTACCCAACTCCAAAAACTCTCGGCAAACAGTCAAAGTGGCCTCT
GTATTTTACACAGTTGTCAACCCCATGCTGAACCCTCCGATCTACAGCCTAAGGAATAAAGACGTGAA
GGATGCTTTCTGGAAGTTAATACATACACAAGTTCCATTTCACTGAACCAGTCTCAAAAGTTGTTTTC
AATCCAAATGAACAACCCA

| | | | |
|---|---|---|---|
| NOV31b, CG53803-01 | SEQ ID NO: 380 | 314 aa | MW at 35790.4kD |
| Protein Sequence | | | |

MMMVLRNLSMEPTFALLGFTDYPKLQIPLFLVFLLMYVITVVGNLGMIIIIKINPKFHTPMYFFLSHL
SFVDFCYSSIVTPKLLENLVMADKSIFYFSCMMQYFLSCTAVVTESFLLAVMAYDRFVAICNPLLYTV
AMSQRLCALLVAGSYLWGMFGPLVLLCYALRLNFSGPNVINHFFCEYTALISVSGSDILIPHLLLFSF
ATFNEMCTLLIILTSYVFIFVTVLKIRSVSGRHKAFSTWASHLTAITIFHGTILFLYCVPNSKNSRQT
VKVASVFYTVVNPMLNPPIYSLRNKDVKDAFWKLIHTQVPFH

A ClustalW comparison of the above protein sequences yields the following sequence alignment shown in Table 31B.

TABLE 31B
Comparison of the NOV31 protein sequences.

NOV31a MMMVLRNLSMEPTFALLGFTDYPKLQIPLFLVFLLMYVITVVGNLGMIIIIKINPKFHTP
NOV31b MMMVLRNLSMEPTFALLGFTDYPKLQIPLFLVFLLMYVITVVGNLGMIIIIKINPKFHTP
NOV31a MYFFLSHLSFVDFCYSSIVTPKLLENLVMADKSIFYFSCMMQYFLSCTAVVTESFLLAVM
NOV31b MYFFLSHLSFVDFCYSSIVTPKLLENLVMADKSIFYFSCMMQYFLSCTAVVTESFLLAVM
NOV31a AYDRFVAICNPLLYTVAMSQRLCALLVAGSYLWGMFGPLVLLCYALRLNFSGPNVINHFF
NOV31b AYDRFVAICNPLLYTVAMSQRLCALLVAGSYLWGMFGPLVLLCYALRLNFSGPNVINHFF
NOV31a CEYTALISVSGSDILIPHLLLFSFATFNEMCTLLIILTSYVFIFVTVLKIRSVSGRHKAF
NOV31b CEYTALISVSGSDILIPHLLLFSFATFNEMCTLLIILTSYVFIFVTVLKIRSVSGRHKAF
NOV31a STWASHLTAITIFHGTILFLYCVPNSKNSRQTVKVASVFYTVVNPMLNPPIYSLRNKDVK
NOV31b STWASHLTAITIFHGTILFLYCVPNSKNSRQTVKVASVFYTVVNPMLNPPIYSLRNKDVK
NOV31a DAFWKLIHTQVPFH
NOV31b DAFWKLIHTQVPFH
NOV31a (SEQ ID NO: 378)
NOV31b (SEQ ID NO: 380)

Further analysis of the NOV31a protein yielded the following properties shown in Table 31C.

TABLE 31C
Protein Sequence Properties NOV31a

| | |
|---|---|
| SignalP analysis: | Cleavage site between residues 44 and 45 |

PSORT II analysis:
PSG: a new signal peptide prediction method

N-region: length 11; pos.chg 1; neg.chg 1
H-region: length 9; peak value 9.41
PSG score: 5.01

GvH: von Heijne's method for signal seq. recognition

GvH score (threshold: −2.1): −1.06
possible cleavage site: between 43 and 44

>>> Seems to have a cleavable signal peptide (1 to 43)
ALOM: Klein et al's method for TM region allocation

| Init position for calculation: 44 | | |
|---|---|---|
| Tentative number of TMS(s) for the threshold 0.5: 5 | | |
| INTEGRAL | Likelihood = −2.23 | Transmembrane 104-120 |
| INTEGRAL | Likelihood = −0.64 | Transmembrane 145-161 |
| INTEGRAL | Likelihood = −0.80 | Transmembrane 186-202 |
| INTEGRAL | Likelihood = −8.39 | Transmembrane 212-228 |
| INTEGRAL | Likelihood = −2.71 | Transmembrane 247-263 |

| | |
|---|---|
| PERIPHERAL | Likelihood = 2.07 (at 63) |

ALOM score: −8.39 (number of TMSs: 5)

MTOP: Prediction of membrane topology (Hartmann et al.)

Center position for calculation: 21
Charge difference: 3.0 C(1.0) − N(−2.0)
C > N: C-terminal side will be inside

>>>Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75): | 7.94 |
| Hyd Moment(95): | 8.95 | G content: | 1 |
| D/E content: | 2 | S/T content: | 3 |
| Score: −5.12 | | | |

Gavel: prediction of cleavage sites for mitochondrial preseq

R-2 motif at 16 LRN|LS

NUCDISC: discrimination of nuclear localization signals

pat4: none
pat7: none
bipartite: none
content of basic residues: 6.4%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***

LLVAGSYLWGMFGPLVLLCYAL at 145

none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

44.4%: endoplasmic reticulum
22.2%: vacuolar
22.2%: mitochondrial
11.1%: Golgi

>> prediction for CG53803-02 is end (k = 9)

A search of the NOV31a protein against the Geneseq database, a proprietary database that contains sequences published in patents and patent publication, yielded several homologous proteins shown in Table 31D.

TABLE 31D
Geneseq Results for NOV31a

| | | NOV31a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85141 | G-coupled olfactory receptor #2 - | 1 . . . 314 | 314/314 (100%) | 0.0 |
| | Homo sapiens, 314 aa. | 1 . . . 314 | 314/314 (100%) | |
| | [WO200198526-A2, 27-DEC- | | | |
| | 2001] | | | |
| AAG72434 | Human OR-like polypeptide | 1 . . . 314 | 314/314 (100%) | 0.0 |
| | query sequence, SEQ ID NO: | 1 . . . 314 | 314/314 (100%) | |
| | 2115 - Homo sapiens, 314 aa. | | | |
| | [WO200127158-A2, 19-APR- | | | |
| | 2001]\| | | | |
| AAG71725 | Human olfactory receptor | 1 . . . 314 | 314/314 (100%) | 0.0 |
| | polypeptide, SEQ ID NO: 1406 - | 1 . . . 314 | 314/314 (100%) | |
| | Homo sapiens, 314 aa. | | | |
| | [WO200127158-A2, 19-APR- | | | |
| | 2001] | | | |
| AAE07540 | Human G-protein coupled | 1 . . . 314 | 314/314 (100%) | 0.0 |
| | receptor 2a (GPCR2a) variant - | 1 . . . 314 | 314/314 (100%) | |
| | Homo sapiens, 314 aa. | | | |
| | [WO200159113-A2, 16-AUG- | | | |
| | 2001] | | | |
| AAU24517 | Human olfactory receptor | 1 . . . 314 | 314/314 (100%) | 0.0 |
| | AOLFR2 - Homo sapiens, 314 | 1 . . . 314 | 314/314 (100%) | |
| | aa. [WO200168805-A2, 20-SEP- | | | |
| | 2001] | | | |

In a BLAST search of public sequence databases, the NOV31a protein was found to have homology to the proteins shown in the BLASTP data in Table 31E.

TABLE 31E
Public BLASTP Results for NOV31a

| | | NOV31a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC69325 | Sequence 3 from Patent | 1 . . . 314 | 314/314 (100%) | 0.0 |
| | WO0159113 - Homo sapiens | 1 . . . 314 | 314/314 (100%) | |
| | (Human), 314 aa. | | | |
| CAC69326 | Sequence 5 from Patent | 1 . . . 314 | 313/314 (99%) | 0.0 |
| | WO0159113 - Homo sapiens | 1 . . . 314 | 313/314 (99%) | |
| | (Human), 314 aa. | | | |
| Q8NGL3 | Seven transmembrane helix | 1 . . . 314 | 312/314 (99%) | 0.0 |
| | receptor - Homo sapiens | 1 . . . 314 | 313/314 (99%) | |
| | (Human), 314 aa. | | | |
| CAD37508 | Sequence 39 from Patent | 10 . . . 314 | 305/305 (100%) | e−177 |
| | WO0224726 - Homo sapiens | 1 . . . 305 | 305/305 (100%) | |
| | (Human), 305 aa. | | | |
| Q8VFR4 | Olfactory receptor MOR174-8 - | 7 . . . 313 | 243/307 (79%) | e−139 |
| | Mus musculus (Mouse), 316 | 7 . . . 313 | 267/307 (86%) | |
| | aa. | | | |

PFam analysis indicates that the NOV31a protein contains the domains shown in the Table 31F.

TABLE 31F
Domain Analysis of NOV31a

| | NOV31a | Identities/ | |
|---|---|---|---|
| | Match | Similarities | |
| Pfam Domain | Region | for the Matched Region | Expect Value |
| 7tm_1 | 43 . . . 292 | 58/278 (21%) | 1.4e−24 |
| | | 174/278 (63%) | |

### Example 32

The NOV32 clone was analyzed, and the nucleotide and encoded polypeptide sequences are shown in Table 32A.

TABLE 32A
NOV32 Sequence Analysis

| | | |
|---|---|---|
| NOV32a, CG53989-03 | SEQ ID NO: 381 | 1092 bp |
| DNA Sequence | ORF Start: ATG at 30 | ORF Stop: TAG at 1047 |

CCGGGACAGTCACCTCCAGCAGCATCCTGATGTTCTGGGGACACTGGTGCTGGGGGGTCAGTGGGGAA
GGGCTCCTGGGTCCTCATGACCCTCTCCCTTGGGTGAGCACAAAACACCATGGCACTTTGGGGTCTTG
GAAACAGACTAAAGGGGATGTCAAGTCCTCATTCCTTGGAGCTGCTGACGAGTCCAGAATGGGTCATG
TTTTCTTGCCCCGACCCCAGCACCTCAGGGCAGCGGAAGGTCCAGAGAGAGGTCGGGGACCGGGGCCG
CTCCTTGCATCCTGGGCTTGTGTCTGTTGCCCCCTGGCTGGTGACTTGCACTCTCCTGGAGCTGGTTC
TTGCAGCCGAGGCCGTCACGGGGCTGGGATGTCGCTGCTGCTTCTCTTCGTGGTGTTGACCATTTCTC
AGACCTCCCCCCGCCCCTGCAGGGAGGAGTTGGAGGCTTGCGCGTTTAGAGTGCAGGTGGGGCAGCTG
AGGCTCTATGAGGACGACCAGCGGACGAAGGTGGTTGAGATCGTCCGTCACCCCCAGTACAACGAGAG
CCTGTCTGCCCAGGGCGGTGCGGACATCGCCCTGCTGAAGCTGGAGGCCCCGGTGCCGCTGTCTGAGC
TCATCCACCCGGTCTCGCTCCCGTCTGCCTCCCTGGACGTGCCCTCGGGGAAGACCTGCTGGGTGACC
GGCTGGGGTGTCATTGGACGTGGAGAACTACTGCCCTGGCCCCTCAGCTTGTGGGAGGCGACGGTGAA
GGTCAGGAGCAACGTCCTCTGTAACCAGACCTGTCGCCGCCGCTTTCCTTCCAACCACACTGAGCGGT
TTGAGCGGCTCATCAAGGACGACATGCTGTGTGCCGGGGACGGGAACCACGGCTCCTGGCCAGGCGAC
AACGGGGGCCCCCTCCTGTGCGGGCGGAATTGCACCTGGGTCCAGGTGGAGGTGGTGAGCTGGGGCAA
ATTCTGCGGCCTTCGCGGCTATCCCGGCATGTACACTCGCGTGACGAGCTACGTGTCCTGGATCCGCC
AGTACGTCCCGCCGTTCCCCAGACGCTAGCTGGGGTGCAGTGGGGTCTGCATGATCCAGGAGGGCCCG
TCTT

| | | | |
|---|---|---|---|
| NOV32a, CG53989-03 | SEQ ID NO: 382 | 339 aa | MW at 37349.5kD |
| Protein Sequence | | | |

MFWGHWCWGVSGEGLLGPHDPLPWVSTKHHGTLGSWKQTKGDVKSSFLGAADESRMGHVFLPRPQHLR
AAEGPERGRGPGPLLASWACVCCPLAGDLHSPGAGSCSRGRHGAGMSLLLLFVVLTISQTSPRPCREE
LEACAFRVQVGQLRLYEDDQRTKVVEIVRHPQYNESLSAQGGADIALLKLEAPVPLSELIHPVSLPSA
SLDVPSGKTCWVTGWGVIGRGELLPWPLSLWEATVKVRSNVLCNQTCRRRFPSNHTERFERLIKDDML
CAGDGNHGSWPGDNGGPLLCGRNCTWVQVEVVSWGKFCGLRGYPGMYTRVTSYVSWIRQYVPPFPRR

| | | |
|---|---|---|
| NOV32b, CG53989-04 | SEQ ID NO: 383 | 881 bp |
| DNA Sequence | ORF Start: ATG at 31 | ORF Stop: TAG at 832 |

GCTCTGGGAAGACCCTCGTCCGTCCCCCTCATGAGCCCGGCACGGGGCGTGAGCTGGTGGGCATCACT
GGGGGCTGCGACGTCCCGGCCAGGAGGCACCCCTGGTAGGGAGGAGTTGGAGGCTTGCGCGTTTAGAG
TGCAGGTGGGGCAGCTGAGGCTCTATGAGGACGACCAGCGGACGAAGGTGGTTGAGATCGTCCGTCAC
CCCCAGTACAACGAGAGCCTGTCTGCCCAGGGCGGTGCGGACATCGCCCTGCTGAAGCTGGAGGCCCC
GGTGCCGCTGTCTGAGCTCATCCACCCGGTCTCGCTCCCGTCTGCCTCCCGGGACGTGCCCTCGGGGA
AGACCTGCTGGGCCTGGGCTCCAGACGCGTCCTGGATGGCTTCCTGCCGCTGCCGAGACGGATGGCCA
CAGGCCAGGTGGCTCAGAGCAGCAGGAATGTACTATCTCACGGCTCTGCAGGCGGAACGTCCACACTC
CAGGCGTGGGCAGGAACTACTGCCCTGGCCCCTCAGCTTGTGGGAGGCGACGGTGAAGGTCAGGAGCA
ACGTCCTCTGTAACCAGACCTGTCGCCGCCGCTTTCCTTCCAACCACACTGAGCGGTTTGAGCGGCTC
ATCAAGGACGACATGCTGTGTGCCGGGGACGGGAACCACGGCTCCTGGCCAGGCGACAACGGGGGCCC
CCTCCTGTGCAGGCGGAATTGCACCTGGGTCCAGGTGGAGGTGGTGAGCTGGGGCAAATTCTGCGGCC
TTCGCGGCTATCCCGGCATGTACACTCGCGTGACGAGCTACGTGTCCTGGATCCGCCAGTACGTCCCG
CCGTTCCCCAGACGCTAGCTGGGGTGCAGTGGGGTCTGCATGATCCAGGAGGGCCCGTCTAAGCG

| | | | |
|---|---|---|---|
| NOV32b, CG53989-04 | SEQ ID NO: 384 | 267 aa | MW at 30126.0kD |
| Protein Sequence | | | |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWAWAPDASWMASCRCRDGWPQARWLRAAGM
YYLTALQAERPHSRRGQELLPWPLSLWEATVKVRSNVLCNQTCRRRFPSNHTERFERLIKDDMLCAGD
GNHGSWPGDNGGPLLCRRNCTWVQVEVVSWGKFCGLRGYPGMYTRVTSYVSWIRQYVPPFPRR

| | | |
|---|---|---|
| NOV32c, 306076095 | SEQ ID NO: 385 | 819 bp |
| DNA Sequence | ORF Start: at 1 | ORF Stop: end of sequence |

CGCAAGCTTATGAGCCCGGCACGGGGCGTGAGCTGGTGGGCATCACTGGGGGCTGCGACGTCTCGGCC
AGGAGGCACCCCTGGCAGGGAGGAGTTGGAGGCTTGCGCGTTTAGAGTGCAGGTGGGGCAGCTGAGGT
TCTATGAGGACGACCAGCGGACGAAGGTGGTTGAGATCGTCCGTCACCCCCAGTACAACGAGAGCCTG
TCTGCCCAGGGCGGTGCGGACATCGCCCTGCTGAAGCTGGAGGCCCCGGTGCCGCTGTCTGAGCTCAT
CCACCCGGTCTCGCTCCCGTCTGCCTCCCGGGACGTGCCCTCGGGGAAGACCTGCTGGGCCTGGGCTC
CAGACGCGTCCTGGATGGCTTCCTGCCGCTGCCGAGACGGATGGCCACAGGCCAGGTGGCTCAGAGCA
GCAGGAATGTACTATCTCACGGCTCTGCAGGCGGAACGTCCACACTCCAGGCGTGGGCAGGAACTACT
GCCCTGGCCCCTCAGCTTGTGGGAGGCGACGGTGAAGGTCAGGAGCAACGTCCTCTGTAACCAGACCT
GTCGCCGCCGCTTTCCTTCCAACCACACTGAGCGGTTTGAGCGGCTCATCAAGGACGACATGCTGTGT
GCCGGGGACGGGAACCACGGCTCCTGGCCAGGCGACAACGGGGGCCCCCTCCTGTGCAGGCGGAATTG
CACCTGGGTCCAGGTGGAGGTGGTGAGCTGGGGCAAACTCTGCGGCCTTCGCGGCTATCCCGGCATGT
ACACCCGCGTGACGAGCTACGTGTCCTGGATCCGCCAGTACGTCCCGCCGTTCCCCAGACGCGTCGAC
GCG

| | | | |
|---|---|---|---|
| NOV32c, 306076095 | SEQ ID NO: 386 | 273 aa | MW at 30808.8kD |
| Protein Sequence | | | |

RKLMSPARGVSWWASLGAATSRPGGTPGREELEACAFRVQVGQLRFYEDDQRTKVVEIVRHPQYNESL
SAQGGADIALLKLEAPVPLSELIHPVSLPSASRDVPSGKTCWAWAPDASWMASCRCRDGWPQARWLRA
AGMYYLTALQAERPHSRRGQELLPWPLSLWEATVKVRSNVLCNQTCRRRFPSNHTERFERLIKDDMLC
AGDGNHGSWPGDNGGPLLCRRNCTWVQVEVVSWGKLCGLRGYPGMYTRVTSYVSWIRQYVPPFPRRVD
A

| | | |
|---|---|---|
| NOV32d, CG53989-01 | SEQ ID NO: 387 | 858 bp |
| DNA Sequence | ORF Start: ATG at 1 | ORF Stop: TAG at 856 |

ATGCTGTGGCTACTGCTCCTGACCCTCCCCTGCCTGATGGGCTCTGTGCCCAGGAACCCAGGCGAGGG
CACGGGGCGTGAGCTGGTGGGCATCACTGGGGGCTGCGACGTCTCGGCCAGGAGGCACCCCTGGCAGG
TCAGCCTGAGGTTCTACAGCATGAAGAAGGGTCTGTGGGAGCCCATCTGTGGGGGCTCCCTCATCCAC
CCAGAGTGGGTGCTGACCGCCGCCCACTGCCTTTTGGAGGAGTTGGAGGCTTGCGCGTTTAGAGTGCA
GGTGGGGCAGCTGAGGCTCTATGAGGACGACCAGCGGACGAAGGTGGTTGAGATCGTCCGTCACCCCC
AGTACAACGAGAGCCTGTCTGCCCAGGGCGGTGCGGACATCGCCCTGCTGAAGCTGGAGGCCCCGGTG
CCGCTGTCTGAGCTCATCCACCCGGTCTCGCTCCCGTCTGCCTCCCTGGACGTGCCCTCGGGGAAGAC
CTGCTGGGTGACCGGCTGGGGTGTCATTGGACGTGGAGAACTACTGCCCTGGCCCCTCAGCTTGTGGG
AGGCGACGGTGAAGGTCAGGAGCAACGTCCTCTGTAACCAGACCTGTCGCCGCCGCTTTCCTTCCAAC
CACACTGAGCGGTTTGAGCGGCTCATCAAGGACGACATGCTGTGTGCCGGGGACGGGAACCACGGCTC
CTGGCCAGGCGACAACGGGGGCCCCCTCCTGTGCAGGCGGAATTGCACCTGGGTCCAGGTGGAGGTGG
TGAGCTGGGGCAAACTCTGCGGCCTTCGCGGCTATCCCGGCATGTACACCCGCGTGACGAGCTACGTG
TCCTGGATCCGCCAGTACGTCCCGCCGTTCCCCAGACGCTAG

| | | | |
|---|---|---|---|
| NOV32d, CG53989-01 | SEQ ID NO: 388 | 285 aa | MW at 31927.6kD |
| Protein Sequence | | | |

MLWLLLLTLPCLMGSVPRNPGEGTGRELVGITGGCDVSARRHPWQVSLRFYSMKKGLWEPICGGSLIH
PENVLTAAHCLLEELEACAFRVQVGQLRLYEDDQRTKVVEIVRHPQYNESLSAQGGADIALLKLEAPV
PLSELIHPVSLPSASLDVPSGKTCWVTGWGVIGRGELLPWPLSLWEATVKVRSNVLCNQTCRRRFPSN
HTERFERLIKDDMLCAGDGNHGSWPGDNGGPLLCRRNCTWVQVEVVSWGKLCGLRGYPGMYTRVTSYV
SWIRQYVPPFPRR

| | | |
|---|---|---|
| NOV32e, CG53989-02 | SEQ ID NO: 389 | 660 bp |
| DNA Sequence | ORF Start: at 1 | ORF Stop: end of sequence |

TCACTGGGGGCTGCGACGTCTCGGCCAGGAGGCACCCCTGGCAGGGAGGAGTTGGAGGCTTGCGCGTT
TAGAGTGCAGGTGGGGCAGCTGAGGCTCTATGAGGACGACCAGCGGACGAAGGTGGTTGAGATCGTCC
GTCACCCCCAGTACAACGAGAGCCTGTCTGCCCAGGGCGGTGCGGACATCGCCCTGCTGAAGCTGGAG
GCCCCGGTGCCGCTGTCTGAGCTCATCCACCCGGTCTCGCTCCCGTCTGCCTCCCGGGACGTGCCCTC
GGGGAAGACCTGCTGGGTGACCGGCTGGGGTGTCATTGGACGTGGAGAACTACTGCCCTGGCCCCTCA
GCTTGTGGGAGGCGACGGTGAAGGTCAGGAGCAACGTCCTCTGTAACCAGACCTGTCGCCGCCGCTTT
CCTTCCAACCACACTGAGCGGTTTGAGCGGCTCATCAAGGACGACATGCTGTGTGCCGGGGACGGGAA
CCACGGCTCCTGGCCAGGCGACAACGGGGGCCCCCTCCTGTGCAGGCGGAATTGCACCTGGGTCCAGG
TGGAGGTGGTGAGCTGGGGCAAACTCTGCGGCCTTCGCGGCTATCCCGGCATGTACACCCGCGTGACG
AGCTACGTGTCCTGGATCCGCCAGTACGTCCCGCCGTTCCCCAGACGC

| | | | |
|---|---|---|---|
| NOV32e, CG53989-02 | SEQ ID NO: 390 | 220 aa | MW at 24527.8kD |
| Protein Sequence | | | |

SLGAATSRPGGTPGREELEACAFRVQVGQLRLYEDDQRTKVVEIVRHPQYNESLSAQGGADIALLKLE
APVPLSELIHPVSLPSASRDVPSGKTCWVTGWGVIGRGELLPWPLSLWEATVKVRSNVLCNQTCRRRF
PSNHTERFERLIKDDMLCAGDGNHGSWPGDNGGPLLCRRNCTWVQVEVVSWGKLCGLRGYPGMYTRVT
SYVSWIRQYVPPFPRR

| | | |
|---|---|---|
| NOV32f, CG53989-05 | SEQ ID NO: 391 | 2847 bp |
| DNA Sequence | ORF Start: ATG at 1 | ORF Stop: TAG at 2845 |

ATGGTCAGCAAGGGGGGAGTTGCTGCAGAGCCAGAGCCACACTATTGTGAGGACAGTGAAAGAGGCCC
CAACACCCTCACAGGTCCGGGCAGCCTTCCTAGAGGAGGTGGCATTGAGGTGGGCATGGAGTTTCCGG
GATGCAGCGGTGAAGGGTGCGTGAAGCCCCATGAGGAGGCGGCCCGGGAGGGGGCGGGCAGAGGCAAG
AGGGCTGTGCCGGGACCCAAGCGACGGCAGCAGGGGTCAGCAGAGGGGCCTGCGGCGGGGTGGACGCT
GGAGCAGGAGACCAGGGGAGATGTCTTAGAGGATAAAAATGAGCGGGCAGATGAAGAGATACTCAGGC
TGGCACCAGGGAAAGGCAGGCTCCCAATAGACAGCAAACACCTGAAACCGGTGATCAGCAGCTTCCCG
GTAAGATCTCAGGAGCTGGGCGAGGGGGCTGGAGCAGGCACACTAAGAGGCAAAATGGCAGAGTTTAA
CTGGTCTATGGCCTTCAAGGGACCTGCGGCTGGTCATGAAGAGCGCCTCAACTCTGTGTCCAGCAGGG
CCAAGAAGGGCATTGGCTGGGATGTCGCTGCTGCTTCTCTTCGTGGTGTTGACCATTTCTCAGACCTC
CCCCCGCCCCTGCAGGTCAGGGAGGAGTTGGAGGCTTGCGCGTTTAGAGTGCAGGTGGGGCAGCTGAG
GCTCTATGAGGACGACCAGCGGACGAAGGTGGTTGAGATCGTCCGTCACCCCCAGTACAACGAGAGCC
TGTCTGCCCAGGGCGGTGCGGACATCGCCCTGCTGAAGCTGGAGGCCCCGGTGCCGCTGTCTGAGCTC
ATCCACCCGGTCTCGCTCCCGTCTGCCTCCCTGGACGTGCCCTCGGGGAAGACCTGCTGGGTGACCGG
CTGGGGTGTCATTGGACGTGGAGAACTACTGCCCTGGCCCCTCAGCTTGTGGGAGGCGACGGTGAAGG
TCAGGAGCAACGTCCTCTGTAACCAGACCTGTCGCCGCCGCTTTCCTTCCAACCACACTGAGCGGTTT
GAGCGGCTCATCAAGGACGACATGCTGTGTGCCGGGGACGGGAACCACGGCTCCTGGCCAGGCGACAA
CGGGGGCCCCCTCCTGTGCAGGCGGAATTGCACCTGGGTCCAGGTGGAGGTGGTGAGCTGGGGCAAAC
TCTGCGGCCTTCGCGGCTATCCCGGCATGTACACCCGCGTGACGAGCTACGTGTCCTGGATCCGCCAG
CCATGCCCCTCAGCTCAGACCCCTGCTGTGGTCCGAAGATTTGTGCTCCCCCCAAATCCAGATGTTGA
AGCCCTAACTCCCAGTGTGATGGGATCAGGAGCGCCGCTGCCCCCGGCCCCCGACCTGCAAGAGGCCG
AGGTCCCCATCATGAGGACCCGAGCTTGCGAGAGGATGTATCACAAAGGCCCCACTGCCCACGGCCAG
GTCACCATCATCAAGGCTGCCATGCCGTGTGCAGGGAGGAAGGGGCAGGGTTCCTGCCAGGCCGCTCT
GAGGACGGAGGACCTCACCCCAACCACACCCAACACGGAGGTGTCTCCACGTGCAGACCCCAGGCTGA
GCCAGCCGGAGGACATCTGGCCAGAGTGGGCTTGGCCAGTTGTGGTGGGCACCACCATGCTGCTGCTG
CTGCTGTTCCTGGCTGTCTCCTCCCTGGGGAGCTGTAGCACTGGGAGTCCAGCTCCCGTCCCCGAGAA
TGACCTGGTGGGCATTGTGGGGGGCCACAACACCCCAGGGGAAGTGGTCGTGGCAGTGGGTGCTGACC
GCCGCTCACTGCATTTTCCGGAAGGACACCGACCCGTCCACCTACCGGATTCACACCAGGGATGTGTA
TCTGTACGGGGGCCGGGGGCTGCTGAATGTCAGCCAGATCGTCGTCCACCCAACTACTCTGTCTTCTT
CCTGGGGGCAGACATCGCCCTGCTGAAGCTGGCCACCAGTTCCCTGGAGTTCACTGACAGTGACAACT
GCTGGAACACAGGCTGGGGCATGGTCGGCTTGTTGGATATGCTGCCGCCTCCTTACCGCCCGCAGCAG
GTGAAGGTCCTCACACTGAGCAATGCAGACTGTGAGCGGCAGACCTACGATGCTTTTCCTGGTGCTGG
AGACAGAAAGTTCATCCAGGATGACATGATCTGTGCCGGCCGCACGGGCCGCCGCACCTGGAAGGGTG
ACTCAGGCGGCCCCCTGGTCTGCAAGAAGAAGGGTACCTGGCTCCAGGCGGGAGTAGTGAGCTGGGGA
TTTTACAGTGATCGGCCCAGCATTGGCGTCTACACACGCCCAGAGACCAGCTGGCAGGGTGCCAACCA
TGCAGACGCCCAGAGACCAGCTGGCAGGGTGCCAACCATGCAGAGGCCCAGAGACATGGGCCAGGGCC
AGGAGTGGGTCTGCAGGCCCTTCACCCACGTCACCTGCTACCCGACGGCCATCCCCAGGCCCTTCACC
CATGTCACCTGCTACCTGATGGCTGTCCCCAGCACCCTCACCCACGTCACCTGCTACCCGACGGCCGT
CCCCAGGCCCTTCACCCATGTCACCTGCTACCTGATGGCTGTCCCCAGCACCCTCACCCATCTCACCT
GCTACATGATGGCCGTCCCCAGGCCCTTTACCCACATCACCTGCTACCCAATGGCTGTCCCCAGCACC
CTTACCCACGTCACCTGCCACCCGACGGCCATCCCCAGGCCCTTCACCCACATCACCTGCTACACGAT
GGCCATCCCCAGGCCTTCAACCACGCCACCTGCTACACGACGGCCATCCCCAGCACCCTCACCCACGT
CACCTGCTACACGATGGCCGTCCCCAGGCCCATCACCCATGTCACCTGCTACACGATAG

| | | | |
|---|---|---|---|
| NOV32f, GG53989-05 | SEQ ID NO: 392 | 948 aa | MW at 102839.3kD |
| Protein Sequence | | | |

MVSKGGVAAEPEPHYCEDSERGPNTLTGPGSLPRGGGIEVGMEFPGCSGEGCVKPHEEAAREGAGRGK
RAVPGPKRRQQGSAEGPAAGWTLEQETRGDVLEDKNERADEEILRLAPGKGRLPIDSKHLKPVISSFP
VRSQELGEGAGAGTLRGKMAEFNWSMAFKGPAAGHEERLNSVSSRAKKGIGWDVAAASLRGVDHFSDL
PPPLQVREELEACAFRVQVGQLRLYEDDQRTKVVEIVRHPQYNESLSAQGGADIALLKLEAPVPLSEL
IHPVSLPSASLDVPSGKTCWVTGWGVIGRGELLPWPLSLWEATVKVRSNVLCNQTCRRRFPSNHTERF
ERLIKDDMLCAGDGNHGSWPGDNGGPLLCRRNCTWVQVEVVSWGKLCGLRGYPGMYTRVTSYVSWIRQ
PCPSAQTPAVVRRFVLPPNPDVEALTPSVMGSGAPLPPAPDLQEAEVPIMRTRACERMYHKGPTAHGQ
VTIIKAAMPCAGRKGQGSCQAALRTEDLTPTTPNTEVSPRADPRLSQPEDIWPEWAWPVVVGTTMLLL
LLFLAVSSLGSCSTGSPAPVPENDLVGIVGGHNTPGEVVVAVGADRRSLHFPEGHRPVHLPDSHQGCV
SVRGPGAAECQPDRRPPNYSVFFLGADIALLKLATSSLEFTDSDNCWNTGWGMVGLLDMLPPPYRPQQ
VKVLTLSNADCERQTYDAFPGAGDRKFIQDDMICAGRTGRRTWKGDSGGPLVCKKKGTWLQAGVVSWG
FYSDRPSIGVYTRPETSWQGANHADAQRPAGRVPTMQRPRDMGQGQEWVCRPFTHVTCYPTAIPRPFT
HVTCYLMAVPSTLTHVTCYPTAVPRPFTHVTCYLMAVPSTLTHITCYMMAVPRPFTHITCYPMAVPST
LTHVTCHPTAIPRPFTHITCYTMAIPRPSTTPPATRRPSPAPSPTSPATRWPSPGPSPMSPATR

| | | |
|---|---|---|
| NOV32g, CG53989-06 | SEQ ID NO: 393 | 660 bp |
| DNA Sequence | ORF Start: at 1 | ORF Stop: end of sequence |

TCACTGGGGGCTGCGACGTCTCGGCCAGGAGGCACCCCTGGCAGGGAGGAGTTGGAGGCTTGCGCGTT
TAGAGTGCAGGTGGGGCAGCTGAGGCTCTATGAGGACGACCAGCGGACGAAGGTGGTTGAGATCGTCC
GTCACCCCCAGTACAACGAGAGCCTGTCTGCCCAGGGCGGTGCGGACATCGCCCTGCTGAAGCTGGAG
GCCCCGGTGCCGCTGTCTGAGCTCATCCACCCGGTCTCGCTCCCGTCTGCCTCCCGGGACGTGCCCTC
GGGGAAGACCTGCTGGGTGACCGGCTGGGGTGTCATTGGACGTGGAGAACTACTGCCCTGGCCCCTCA
GCTTGTGGGAGGCGACGGTGAAGGTCAGGAGCAACGTCCTCTGTAACCAGACCTGTCGCCGCCGCTTT
CCTTCCAACCACACTGAGCGGTTTGAGCGGCTCATCAAGGACGACATGCTGTGTGCCGGGGACGGGAA
CCACGGCTCCTGGCCAGGCGACAACGGGGGCCCCCTCCTGTGCAGGCGGAATTGCACCTGGGTCCAGG
TGGAGGTGGTGAGCTGGGGCAAACTCTGCGGCCTTCGCGGCTATCCCGGCATGTACACCCGCGTGACG
AGCTACGTGTCCTGGATCCGCCAGTACGTCCCGCCGTTCCCCAGACGC

| | | | |
|---|---|---|---|
| NOV32g, CG53989-06 | SEQ ID NO: 394 | 220 aa | MW at 24527.8kD |
| Protein Sequence | | | |

SLGAATSRPGGTPGREELEACAFRVQVGQLRLYEDDQRTKVVEIVRHPQYNESLSAQGGADIALLKLE
APVPLSELIHPVSLPSASRDVPSGKTCWVTGWGVIGRGELLPWPLSLWEATVKVRSNVLCNQTCRRRF
PSNHTERFERLIKDDMLCAGDGNHGSWPGDNGGPLLCRRNCTWVQVEVVSWGKLCGLRGYPGMYTRVT
SYVSWIRQYVPPFPRR

| | | |
|---|---|---|
| NOV32h, CG53989-07 | SEQ ID NO: 395 | 672 bp |
| DNA Sequence | ORF Start: at 7 | ORF Stop: at 667 |

AGATCTTCACTGGGGGCTGCGACGTCTCGGCCAGGAGGCACCCCTGGCAGGGAGGAGTTGGAGGCTTG
CGCGTTTAGAGTGCAGGTGGGGCAGCTGAGGCTCTATGAGGACGACCAGCGGACGAAGGTGGTTGAGA
TCGTCCGTCACCCCCAGTACAACGAGAGCCTGTCTGCCCAGGGCGGTGCGGACATCGCCCTGCTGAAG
CTGGAGGCCCCGGTGCCGCTGTCTGAGCTCATCCACCCGGTCTCGCTCCCGTCTGCCTCCCTGGACGT
GCCCTCGGGGAAGACCTGCTGGGTGACCGGCTGGGGTGTCATTGGACGTGGAGAACTACTGCCCTGGC
CCCTCAGCTTGTGGGAGGCGACGGTGAAGGTCAGGAGCAACGTCCTCTGTAACCAGACCTGTCGCCGC
CGCTTTCCTTCCAACCACACTGAGCGGTTTGAGCGGCTCATCAAGGACGACATGCTGTGTGCCGGGGA
CGGGAACCACGGCTCCTGGCCAGGCGACAACGGGGGCCCCCTCCTGTGCAGGCGGAATTGCACCTGGG
TCCAGGTGGAGGTGGTGAGCTGGGGCAAACTCTGCGGCCTTCGCGGCTATCCCGGCATGTACACCCGC
GTGACGAGCTACGTGTCCTGGATCCGCCAGTACGTCCCGCCGTTCCCCAGACGCCTCGAG

| | | | |
|---|---|---|---|
| NOV32h, CG53989-07 | SEQ ID NO: 396 | 220 aa | MW at 24484.8kD |
| Protein Sequence | | | |

SLGAATSRPGGTPGREELEACAFRVQVGQLRLYEDDQRTKVVEIVRHPQYNESLSAQGGADIALLKLE
APVPLSELIHPVSLPSASRDVPSGKTCWVTGWGVIGRGELLPWPLSLWEATVKVRSNVLCNQTCRRRF
PSNHTERFERLIKDDMLCAGDGNHGSWPGDNGGPLLCRRNCTWVQVEVVSWGKLCGLRGYPGMYTRVT
SYVSWIRQYVPPFPRR

| | | |
|---|---|---|
| NOV32i,CG53989-08 | SEQ ID NO: 397 | 1171 bp |
| DNA Sequence | ORF Start: at 187 | ORF Stop: TGA at 805 |

ATGCTGTGGCTACTGCTCCTGACCCTCCCCTGCCTGATGGGCTCTGTGCCCAGGAACCCAGGCGAGTC
CGCCCCACCCAATGCCCCTGCTGCCCAGGACCCCCTCCTTGCCCTGCCCCGGGCTCAGAGTGCCAGCC
CTGGGGTGGGTGGGGACCATCTGATTGCCGGTCTCTCCTGGTGCCCCTGAGCTCTGGGAAGACCCTCG
TCCGTCCCCCTCATGAGCCCGGCACGGGGCGTGAGCTGGTGGGCATCACTGGGGGCTGCGACGTCTCG
GCCAGGAGGCACCCCTGGCAGGGAGGAGTTGGAGGCTTGCGCGTTTAGAGTGCAGGTGGGGCAGCTGA
GGCTCTATGAGGACGACCAGCGGACGAAGGTGGTTGAGATCGTCCGTCACCCCCAGTACAACGAGAGC
CTGTCTGCCCAGGGCGGTGCGGACATCGCCCTGCTGAAGCTGGAGGCCCCGGTGCCGCTGTCTGAGCT
CATCCACCCGGTCTCGCTCCCGTCTGCCTCCCTGGACGTGCCCTCGGGGAAGACCTGCTGGGTGACCG
GCTGGGGTGTCATTGGACGTGGAGGTCAGGAGCAGGACCACTTGGGTGGGATGTGGAGAGATGACCCG
GAATGTCGGTGCAGGCCTGGGCTCCAGACGCGTCCTGGATGGCTTCCTGCCGCTGCCGAGACGGATGG
CCACAGGCCAGGTGGCTCAGAGCAGCAGGAATGTACTATCTCACGGCTCTGCAGGCGGAACGTCCACA
CTCCAGGCGTGGGCAGGAACTACTGCCCTGGCCCCTCAGCTTGTGGGAGGCGACGGTGAAGGTCAGGA
GCAACGTCCTCTGTAACCAGACCTGTCGCCGCCGCTTTCCTTCCAACCACACTGAGCGGTTTGAGCGG
CTCATCAAGGACGACATGCTGTGTGCCGGGGACGGGAACCACGGCTCCTGGCCAGGCGACAACGGGGG
CCCCCTCCTGTGCAGGCGGAATTGCACCTGGGTCCAGGTGGAGGTGGTGAGCTGGGGCAAACTCTGCG
GCCTTCGCGGCTATCCCGGCATGTACACCCGCGTGACGAGCTACGTGTCCTGGATCCGCCAGTACGTC
CCGCCGTTCCCCAGACGCTAGCTGGGGTGCAGTGGGGTCTGCATGATCCAGGAGGGCCCGTCTTCCTT
GTGGACACGCCTGCT

| | | | |
|---|---|---|---|
| NOV32i, CG53989-08 | SEQ ID NO: 398 | 206 aa | MW at 22191.8kD |
| Protein Sequence | | | |

ALGRPSSVPLMSPARGVSWWASLGAATSRPGGTPGREELEACAFRVQVGQLRLYEDDQRTKVVEIVRH
PQYNESLSAQGGADIALLKLEAPVPLSELIHPVSLPSASLDVPSGKTCWVTGWGVIGRGGQEQDHLGG
MWRDDPECRCRPGLQTRPGWLPAAAETDGHRPGGSEQQECTISRLCRRNVHTPGVGRNYCPGPSACGR
RR

| | | |
|---|---|---|
| NOV32j, CG53989-09 | SEQ ID NO: 399 | 843 bp |
| DNA Sequence | ORF Start: ATG at 26 | ORF Stop: TAG at 836 |

GAGGTGGAGGTTGCAGTAAGCCAAGATGGCGCCACTGCACTCTAGCCTGTTTCTGCTGAGCGGGACCA
TGAGCCCAAAAGTGGGCATCACTGGGGGCTGCGACGTCTCGGCCAGGAGGCACCCCTGGCAGGTCAGC
CTGAGGTTCTACAGCATGAAGAAGGGTCTGTGGGAGCCCATCTGTGGGGGCTCCCTCATCCACCCAGA
GTGGGTGCTGACCGCCGCCCACTGCGTCGAGCTTGAGGAGTTGGAGGCTTGCGCGTTTAGAGTGCAGG
TGGGGCAGCTGAGGCTCTATGAGGACGACCAGCGGACGAAGGTGGTTGAGATCGTCCGTCCCCCCCAG
TACAACGAGAGCCTGTCTGCCCAGGGCGGTGCGGACATCGCCCTGCTGAAGCTGGAGGCCCCGGTGCC
GCTGTCTGAGCTCATCCACCCGGTCTCGCTCCCGTCTGCCTCCCTGGACGTGCCCTCGGGGAAGACCT
GCTGGGTGACCGGCTGGGGTGTCATTTGGGGACACGTTTTCCTGCTCCCGCCACCCCACCTCAGGGCA
GCGGAAGGTCCAATCATGAGGACCCGAGCTTGCGAGAGGATGTATCACAAAGGCCCCACTGCCCACGT
CACCATCATCAAGGCTGCCATGCCGTGTGCAGGGGCTGAGCGCCATCTCTCCCCACAGGGCGACAACG
GGGGCCCCCTCCTGTGCAGGCGGAATTGCACCTGGGTCCAGGTGGAGGTGGTGAGCTGGGGCAAACTC
TGCGGCCTTCGCTATCCCGGCATGTACACCCGCGTGACGAGCTACGTGTCCTGGATCCGCCAGTACGT
CCCGCCGTTCCCCAGACGCTAGCTGGG

| | | | |
|---|---|---|---|
| NOV32j, CG53989-09 | SEQ ID NO: 400 | 270 aa | MW at 29993.6kD |
| Protein Sequence | | | |

MAPLHSSLFLLSGTMSPKVGITGGCDVSARRHPWQVSLRFYSMKKGLWEPICGGSLIHPEWVLTAAHC
VELEELEACAFRVQVGQLRLYEDDQRTKVVEIVRHPQYNESLSAQGGADIALLKLEAPVPLSELIHPV
SLPSASLDVPSGKTCWVTGWGVIWGHVFLLPPPHLRAAEGPIMRTRACERMYHKGPTAHVTIIKAAMP
CAGAERHLSPQGDNGGPLLCRRNCTWVQVEVVSWGKLCGLRYPGMYTRVTSYVSWIRQYVPPFPRR

| | | |
|---|---|---|
| NOV32k, CG53989-10 | SEQ ID NO: 401 | 964 bp |
| DNA Sequence | ORF Start: ATG at 67 | ORF Stop: TGA at 655 |

GACCATCTGATTGCCGGTCTCTCCTGGTGCCCCTGAGCTCTGGGAAGACCCTCGTCCGTCCCCCTCAT
GAGCCCGGCACGGGGCGTGAGCTGGTGGGCATCACTGGGGGCTGCGACGTCTCGGCCAGGAGGCACCC
CTGGCAGGGAGGAGTTGGAGGCTTGCGCGTTTAGAGTGCAGGTGGGGCAGCTGAGGCTCTATGAGGAC
GACCAGCGGACGAAGGTGGTTGAGATCGTCCGTCACCCCCAGTACAACGAGAGCCTGTCTGCCCAGGG
CGGTGCGGACATCGCCCTGCTGAAGCTGGAGGCCCCGGTGCCGCTGTCTGAGCTCATCCACCCGGTCT
CGCTCCCGTCTGCCTCCCGGGACGTGCCCTCGGGGAAGACCTGCTGGGTGACCGGCTGGGGTGTCATT
GGACGTGGAGGTCAGGAGCAGGACCACTTGGGTGGGATGTGGAGAGATGACCCGGAATGTCGGTGCAG
GCCTGGGCTCCAGACGCGTCCTGGATGGCTTCCTGCCGCTGCCGAGACGGATGGCCACAGGCCAGGTG
GCTCAGAGCAGCAGGAATGTACTATCTCACGGCTCTGCAGGCGGAACGTCCACACTCCAGGCGTGGGC
AGGAACTACTGCCCTGGCCCCTCAGCTTGTGGGAGGCGACGGTGAAGGTCAGGAGCAACGTCCTCTGT
AACCAGACCTGTCGCCGCCGCATTCCTTCCAACCACACTGAGCGGTTTGAGCGGCTCATCAAGGACGA
CATGCTGTGTGCCGGGGACGGGAACCACGGCTCCTGGCCAGGCGACAACGGGGGCCCCCTCCTGTGCA
GGCGGAATTGCACCTGGGTCCAGGTGGAGGTGGTGAGCTGGGGCAAACTCTGCGGCCTTCGCGGCTAT
CCCGGCATGTACACCCGCGTGACGAGCTACGTGTCCTGGATCCGCCAGTACGTCCCGCCGTTCCCCAG
ACGCTAGCTGGG

| | | | |
|---|---|---|---|
| NOV32k, CG53989-10 | SEQ ID NO: 402 | 196 aa | MW at 21256.7kD |
| Protein Sequence | | | |

MSPARGVSWWASLGAATSRPGGTPGREELEACAFRVQVGQLRLYEDDQRTKVVEIVRHPQYNESLSAQ
GGADIALLKLEAPVPLSELIHPVSLPSASRDVPSGKTCWVTGWGVIGRGGQEQDHLGGMWRDDPECRC
RPGLQTRPGWLPAAAETDGHRPGGSEQQECTISRLCRRNVHTPGVGRNYCPGPSACGRRR

| | | |
|---|---|---|
| NOV321, CG53989-11 | SEQ ID NO: 403 | 948 bp |
| DNA Sequence | ORF Start: ATG at 61 | ORF Stop: TAG at 931 |

TGACCCTCCCCTGCCTGATGGGCTCTGTGCCCAGGAACCCAGGCGAGTCCGCCCCACCCAATGCCCCT
GCTGCCCAGCCGGTCTCTCCTGGTGCCCCTGAGCTCTGGGAAGACCCTCGTCCGTCCCCCTCATGAGC
CCGGCACGGGGCGTGAGCTGGTGGGCATCACTGGGGGCTGCGACGTCTCGGCCAGGAGGCACCCCTGG
CAGGTCAGCCTGAGGTTCTACAGCATGAAGAAGGGTCTGTGGGAGCCCATCTGTGGGGGCTCCCTCAT
CCACCCAGAGTGGGTGCTGACCGCCGCCCACTGCCTTGGGCCTGAGGAGTTGGAGGCTTGCGCGTTTA
GAGTGCAGGTGGGGCAGCTGAGGCTCTATGAGGACGACCAGCGGACGAAGGTGGTTGAGATCGTCCGT
CACCCCCAGTACAACGAGAGCCTGTCTGCCCAGGGCGGTGCGGACATCGCCCTGCTGAAGCTGGAGGC
CCCGGTGCCGCTGTCTGAGCTCATCCACCCGGTCTCGCTCCCGTCTGCCTCCCTGGACGTGCCCTCGG
GGAAGACCTGCTGGGTGACCGGCTGGGGTGTCATTGGACGTGGAGAACTACTGCCCTGGCCCCTCAGC
TTGTGGGAGGCGACGGTGAAGGTCAGGAGCAACGTCCTCTGTAACCAGACCTGTCGCCGCCGCTTTCC
TTCCAACCACACTGAGCGGTTTGAGCGGCTCATCAAGGACGACATGCTGTGTGCCGGGGACGAGCGCC
ATCTCTCCCCACAGGGCGACAACGGGGGCCCCCTCCTGTGCAGGCGGAATTGCACCTGGGTCCAGGTG
GAGGTGGTGAGCTGGGGCAAACTCTGCGGCGTTCGCGGCTATCCCGGCATGTACACCCGCGTGACGAG
CTACGTGTCCTGGATCCGCCAGTACGTCCCGCCGTTCCCCAGACGCTAGCTGGGGTGCAGTGGG

| | | | |
|---|---|---|---|
| NOV32l, CG53989-11 | SEQ ID NO: 404 | 290 aa | MW at 32449.2kD |
| Protein Sequence | | | |

MPLLPSRSLLVPLSSGKTLVRPPHEPGTGRELVGITGGCDVSARRHPWQVSLRFYSMKKGLWEPICGG
SLIHPEWVLTAAHCLGPEELEACAFRVQVGQLRLYEDDQRTKVVEIVRHPQYNESLSAQGGADIALLK
LEAPVPLSELIHPVSLPSASLDVPSGKTCWVTGWGVIGRGELLPWPLSLWEATVKVRSNVLCNQTCRR
RFPSNHTERFERLIKDDMLCAGDERHLSPQGDNGGPLLCRRNCTWVQVEVVSWGKLCGLRGYPGMYTR
VTSYVSWIRQYVPPFPRR

| | | |
|---|---|---|
| NOV32m, CG53989-12 | SEQ ID NO: 405 | 843 bp |
| DNA Sequence | ORF Start: ATG at 11 | ORF Stop: TAG at 836 |

TGAGAGATAAATGGGCTCCCAGAGATGCCAGGGAGGAGGCCCCGGCACGGGGCGTGAGCTGGTGGGCA
TCACTGGGGGCTGCGACGTCTCGGCCAGGAGGCACCCCTGGCAGGTCAGCCTGAGGTTCTACAGCATG
AAGAAGGGTCTGTGGGAGCCCATCTGTGGGGGCTCCCTCATCCACCCAGAGTGGGTGCTGACCGCCGC
CCACTGCCTTGGCAGGGAGGAGTTGGAGGCTTGCGCGTTTAGAGTGCAGGTGGGGCAGCTGAGGCTCT
ATGAGGACGACCAGCGGACGAAGGTGGTTGAGATCGTCCGTCACCCCCAGTACAACGAGAGCCTGTCT
GCCCAGGGCGGTGCGGACATCGCCCTGCTGAAGCTGGAGGCCCCGGTGCCGCTGTCTGAGCTCATCCA
CCCGGTCTCGCTCCCGTCTGCCTCCCGGCCTGGGCTCCAGACGCGTCCTGGATGGCTTCCTGCCGCTG
CCGAGACGGATGGGCAGGAACTACTGCCCTGGCCCCTCAGCTTGTGGGAGGCGACGGTGAAGGTCAGG
AGCAACGTCCTCTGTAACCAGACCTGTCGCCGCCGCTTTCCTTCCAACCACACTGAGCGGTTTGAGCG
GCTCATCAAGGACGACATGCTGTGTGCCGGGGACGGGAACCACGGCTCCTGGCCAGGCGACAACGGGG
GCCCCCTCCTGTGCAGGCGGAATTGCACCTGGGTCCAGGTGGAGGTGGTGAGCTGGGGCAAACTCTGC
GGCCTTCGCGGCTATCCCGGCATGTACACCCGCGTGACGAGCTACGTGTCCTGGATCCGCCAGTACGT
CCCGCCGTTCCCCAGACGCTAGCTGGG

| | | | |
|---|---|---|---|
| NOV32m, CG53989-12 | SEQ ID NO: 406 | 275 aa | MW at 30683.8kD |
| Protein Sequence | | | |

MGSQRCQGGGPGTGRELVGITGGCDVSARRHPWQVSLRFYSMKKGLWEPICGGSLIHPEWVLTAAHCL
GREELEACAFRVQVGQLRLYEDDQRTKVVEIVRHPQYNESLSAQGGADIALLKLEAPVPLSELIHPVS
LPSASRPGLQTRPGWLPAAAETDGQELLPWPLSLWEATVKVRSNVLCNQTCRRRFPSNHTERFERLIK
DDMLCAGDGNHGSWPGDNGGPLLCRRNCTWVQVEVVSWGKLCGLRGYPGMYTRVTSYVSWIRQYVPPF
PRR

| | | |
|---|---|---|
| NOV32n, CG53989-13 | SEQ ID NO: 407 | 870 bp |
| DNA Sequence | ORF Start: ATG at 43 | ORF Stop: TAA at 868 |

ATCTGGCCAGAGTGGGCTTGGCCAGTTGTGGTGGGCACCACCATGCTGCTGCTGCTGCTGTTCCTGGC
TGTCTCCTCCCTGGGGAGCTGTAGCACTGGGAGTCCAGCTCCCGTCCCCGAGAATGACCTGGTGGGCA
TTGTGGGGGGCCACAACACCCAGGGGAAGTGGTCGTGGCAGGTCAGCCTGAGGATCTATAGCTACCAC
TGGGCCTCCTGGGTGCCCATCTGCGGGGGCTCCCTCATCCACCCCCAGTGGGTGCTGACCGCCGCTCA
CTGCATTTTCCGGAAGGACACCGACCCGTCCACCTACCGGATTCACACCAGGGATGTGTATCTGTACG
GGGGCCGGGGGCTGCTGAATGTCAGCCAGATCGTCGTCCACCCCAACTACTCTGTCTTCTTCCTGGGG
GCAGACATCGCCCTGCTGAAGCTGGCCACCAGTGTGAGAACAACAAACACTCTCGCGGCAGTCGCCCT
GCCGTCATTGTCCCTGGAGTTCACTGACAGTGACAACTGCTGGAACACAGGCTGGGGCATGGTCGGCT
TGTTGGATATGCTGCCGCCTCCTTACCGCCCGCAGCAGGTGAAGGTCCTCACACTGAGCAATGCAGAC
TGTGAGCGGCAGACCTACGATGCTTTTCCTGGTGCTGGAGACAGAAAGTTCATCCAGGATGACATGAT
CTGTGCCGGCCGCACGGGCCGCCGCACCTGGAAGGGTGACTCAGGCGGCCCCCTGGTCTGCAAGAAGA
AGGGTACCTGGCTCCAGGCGGGAGTAGTGAGCTGGGGATTTTACAGTGATCGGCCCAGCATTGGCGTC
TACACGTGGGTCCAGACCTATGTGCCCTGGATCCTGCAGCAAATGCACCTCTAA

| | | | |
|---|---|---|---|
| NOV32n, CG53989-13 | SEQ ID NO: 408 | 275 aa | MW at 30467.7kD |
| Protein Sequence | | | |

MLLLLLFLAVSSLGSCSTGSPAPVPENDLVGIVGGHNTQGKWSWQVSLRIYSYHWASWVPICGGSLIH
PQWVLTAAHCIFRKDTDPSTYRIHTRDVYLYGGRGLLNVSQIVVHPNYSVFFLGADIALLKLATSVRT
TNTLAAVALPSLSLEFTDSDNCWNTGWGMVGLLDMLPPPYRPQQVKVLTLSNADCERQTYDAFPGAGD
RKFIQDDMICAGRTGRRTWKGDSGGPLVCKKKGTWLQAGVVSWGFYSDRPSIGVYTWVQTYVPWILQQ
MHL

A ClustalW comparison of the above protein sequences yields the following sequence alignment shown in Table 32B.

TABLE 32B
Comparison of the NOV32 protein sequences.

NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f MVSKGGVAAEPEPHYCEDSERGPNTLTGPGSLPRGGGIEVGMEFPGCSGEGCVKPHEEAA
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ---------------MFWGMWCWGVSGEGLLGPHDPLPWVSTKHHGTLGSWKQTKGDVKS
NOV32b ------------------------------------------------------------
NOV32c ------------------------------------------------------------
NOV32d --------------------------------------------------MLWLLLLTLP
NOV32e ------------------------------------------------------------
NOV32f REGAGRGKRAVPGPKRRQQGSAEGPAAGWTLEQETRGDVLEDKNERADEEILRLAPGKGR
NOV32g ------------------------------------------------------------
NOV32h ------------------------------------------------------------
NOV32i ------------------------------------------------------------
NOV32j -------------------------------------------------------MAPLH
NOV32k ------------------------------------------------------------
NOV32l -----------------------------------------------------MPLLPSR
NOV32m -------------------------------------------------------MGSQR
NOV32n --------------------------------------------------------MLLL
NOV32a SFLGAADESRMGHVFLPRPQHLRAAEGPERGRGPGPLLASWACVCCPLAGDLHSPGAGSC
NOV32b -------------------------MSPARG------VSWWA-----------SLGAATS
NOV32c ----------------------RKLMSPARG------VSWWA-----------SLGAATS
NOV32d CLMGSVPRNPG-------EGTGRELVGITGGCDVSARRHPWQVS---LR--FYSMKKGLW
NOV32e -----------------------------------------------------SLGAATS
NOV32f LPIDSKHLKPVISSFPVRSQELGEGAGAGTLRGKMAEFNWSMAFKGPAAGHEERLNSVSS
NOV32g -----------------------------------------------------SLGAATS
NOV32h -----------------------------------------------------SLGAATS
NOV32i --------------ALGRPS-SVPLMSPARG------VSWWA-----------SLGAATS
NOV32j SSLFLLS------------GTMSPKVGITGGCDVSARRHPWQVS---LR--FYSMKKGLW
NOV32k -------------------------MSPARG------VSWWA-----------SLGAATS
NOV32l SLLVPLSSGKTLVRPPHEPGTGRELVGITGGCDVSARRHPWQVS---LR--FYSMKKGLW
NOV32m CQGG--G-----------PGTGRELVGITGGCDVSARRHPWQVS---LR--FYSMKKGLW
NOV32n LLFLAVSSLGSCSTGSPAPVPENDLVGIVGGHNTQG-KWSWQVSLR-----IYSYHWASW
NOV32a SRGR-HGAGMSLLLLFVVLTISQTSPRPC-REELEACAFRVQVGQLRLYEDDQRTKVVEI
NOV32b RPG---G-----------------TPG---REELEACAFRVQVGQLRLYEDDQRTKVVEI
NOV32c RPG---G-----------------TPG---REELEACAFRVQVGQLRLYEDDQRTKVVEI
NOV32d EPIC-GG-------SLIHPEWVLTAAHCL-LEELEACAFRVQVGQLRLYEDDQRTKVVEI
NOV32e RPG---G-----------------TPG---REELEACAFRVQVGQLRLYEDDQRTKVVEI
NOV32f RAKKGIGWDVAAASLRGVDHFSDLPPPLQVREELEACAFRVQVGQLRLYEDDQRTKVVEI
NOV32g RPG--------------------GTPG---REELEACAFRVQVGQLRLYEDDQRTKVVEI
NOV32h RPG--------------------GTPG---REELEACAFRVQVGQLRLYEDDQRTKVVEI
NOV32i RPG---G-----------------TPG---REELEACAFRVQVGQLRLYEDDQRTKVVEI
NOV32j EPIC-GG-------SLIHPEWVLTAAHCVELEELEACAFRVQVGQLRLYEDDQRTKVVEI
NOV32k RPG---G-----------------TPG---REELEACAFRVQVGQLRLYEDDQRTKVVEI
NOV32l EPIC-GG-------SLIHPEWVLTAAHCLGPEELEACAFRVQVGQLRLYEDDQRTKVVEI
NOV32m EPIC-GG-------SLIHPEWVLTAAHCLGREELEACAFRVQVGQLRLYEDDQRTKVVEI
NOV32n VPIC-GG-------SLIHPQWVLTAAHCIFRKDTDPSTYRIHTRDVYLYGGRGLLNVSQI
NOV32a VRHPQYNESLSAQGGADIALLKLEAPVPLSELIHPVSLPSASLDVPSGKTCWVTG----W
NOV32b VRHPQYNESLSAQGGADIALLKLEAPVPLSELIHPVSLPSASRDVPSGKTCWAWAPDASW
NOV32c VRHPQYNESLSAQGGADIALLKLEAPVPLSELIHPVSLPSASRDVPSGKTCWAWAPDASW
NOV32d VRHPQYNESLSAQGGADIALLKLEAPVPLSELIHPVSLPSASLDVPSGKTCWVTG----W
NOV32e VRHPQYNESLSAQGGADIALLKLEAPVPLSELIHPVSLPSASRDVPSGKTCWVTG----W
NOV32f VRHPQYNESLSAQGGADIALLKLEAPVPLSELIHPVSLPSASLDVPSGKTCWVTG----W
NOV32g VRHPQYNESLSAQGGADIALLKLEAPVPLSELIHPVSLPSASLDVPSGKTCWVTG----W
NOV32h VRHPQYNESLSAQGGADIALLKLEAPVPLSELIHPVSLPSASLDVPSGKTCWVTG----W
NOV32i VRHPQYNESLSAQGGADIALLKLEAPVPLSELIHPVSLPSASLDVPSGKTCWVTG----W
NOV32j VRHPQYNESLSAQGGADIALLKLEAPVPLSELIHPVSLPSASLDVPSGKTCWVTG----W
NOV32k VRHPQYNESLSAQGGADIALLKLEAPVPLSELIHPVSLPSASRDVPSGKTCWVTG----W
NOV32l VRHPQYNESLSAQGGADIALLKLEAPVPLSELIHPVSLPSASLDVPSGKTCWVTG----W
NOV32m VRHPQYNESLSAQGGADIALLKLEAPVPLSELIHPVSLPSASRPGLQTRPGWLPA----A
NOV32n VVHPNYSVFF---LGADIALLKLATSVRTTNTLAAVALPSLSLEFTDSDNCWNTG----W
NOV32a GVIG--R-G--------ELLPWP-----------------------LSLWEATVKVRSNV
NOV32b MASCRCRDGWPQARWLRAAGMYYLTALQAERPHSR-R-GQELLPWPLSLWEATVKVRSNV
NOV32c MASCRCRDGWPQARWLRAAGMYYLTALQAERPHSR-R-GQELLPWPLSLWEATVKVRSNV
NOV32d GVIG----R-------GELLPWP-----------------------LSLWEATVKVRSNV
NOV32e GVIG--R-G--------ELLPWP-----------------------LSLWEATVKVRSNV
NOV32f GVIG---RG--------ELLPWP-----------------------LSLWEATVKVRSNV
NOV32g GVIG--R-G--------ELLPWP-----------------------LSLWEATVKVRSNV
NOV32h GVIG--R-G--------ELLPWP-----------------------LSLWEATVKVRSNV
NOV32i GVIG--RGGQEQD---HLGGMWR------DDPECRCRPGLQTRPGWLPAAAETDGHRPGG
NOV32j GVIW----G-------HVFLLPP-----------------------PHLRAAEGPIMR--
NOV32k GVIG--RGGQEQD---HLGGMWR------DDPECRCRPGLQTRPGWLPAAAETDGHRPGG
NOV32l GVIG----R-------GELLPWP-----------------------LSLWEATVKVRSNV
NOV32m AETD----G-------QELLPWP-----------------------LSLWEATVKVRSNV
NOV32n GMVG----L---------LDMLP-----------------------PPYRPQQVKVLT--
NOV32a LCNQTCRRRFPSNHTERFER-LIKDDMLCAGDGNHGSWPGDNGGPLLCGRNCTWVQVEVV
NOV32b LCNQTCRRRFPSNHTERFER-LIKDDMLCAGDGNHGSWPGDNGGPLLCGRNCTWVQVEVV
NOV32c LCNQTCRRRFPSNHTERFER-LIKDDMLCAGDGNHGSWPGDNGGPLLCGRNCTWVQVEVV
NOV32d LCNQTCRRRFPSNHTERFER-LIKDDMLCAGDGNHGSWPGDNGGPLLCGRNCTWVQVEVV
NOV32e LCNQTCRRRFPSNHTERFER-LIKDDMLCAGDGNHGSWPGDNGGPLLCGRNCTWVQVEVV
NOV32f LCNQTCRRRFPSNHTERFER-LIKDDMLCAGDGNHGSWPGDNGGPLLCGRNCTWVQVEVV
NOV32g LCNQTCRRRFPSNHTERFER-LIKDDMLCAGDGNHGSWPGDNGGPLLCGRNCTWVQVEVV
NOV32h LCNQTCRRRFPSNHTERFER-LIKDDMLCAGDGNHGSWPGDNGGPLLCGRNCTWVQVEVV
NOV32i SEQQECT----------ISR-LCRRNVHTPGVG-RNYCPG----PSACGRRR--------
NOV32j --TRACERMYHKGPTAHVT--IIKAAMPCAGAERHLSPQGDNGGPLLCRRNCTWVQVEVV
NOV32k SEQQECT----------ISR-LCRRNVHTPGVG-RNYCPG----PSACGRRR--------
NOV32l LCNQTCRRRFPSNHTERFER-LIKDDMLCAGDERHLSPQGDNGGPLLCRRNCTWVQVEVV
NOV32m LCNQTCRRRFPSNHTERFER-LIKDDMLCAGDGNHGSWPGDNGGPLLCGRNCTWVQVEVV
NOV32n LSNADCERQTYDAFPGAGDRKFIQDDMICAGRTGRRTWKGDSGGPLVCKKKGTWLQAGVV
NOV32a SWGKFCGLRGYPGMYTRVTSYVSWIRQYVPPFPRR-------------------------
NOV32b SWGKFCGLRGYPGMYTRVTSYVSWIRQYVPPFPRR-------------------------
NOV32c SWGKLCGLRGYPGMYTRVTSYVSWIRQYVPPFPRRVDA----------------------
NOV32d SWGKLCGLRGYPGMYTRVTSYVSWIRQYVPPFPRR-------------------------
NOV32e SWGKLCGLRGYPGMYTRVTSYVSWIRQYVPPFPRR-------------------------
NOV32f SWGKLCGLRGYPGMYTRVTSYVSWIRQPCPSAQTPAVVRRFVLPPNPDVEALTPSVMGSG
NOV32g SWGKLCGLRGYPGMYTRVTSYVSWIRQYVPPFPRR-------------------------
NOV32h SWGKLCGLRGYPGMYTRVTSYVSWIRQYVPPFPRR-------------------------
NOV32i ------------------------------------------------------------
NOV32j SWGKLCGLR-YPGMYTRVTSYVSWIRQYVPPFPRR-------------------------
NOV32k ------------------------------------------------------------
NOV32l SWGKLCGLRGYPGMYTRVTSYVSWIRQYVPPFPRR-------------------------
NOV32m SWGKLCGLRGYPGMYTRVTSYVSWIRQYVPPFPRR-------------------------
NOV32n SWGFYSDRP-SIGVYTWVQTYVPWILQQMHL-----------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f APLPPAPDLQEAEVPIMRTRACERMYHKGPTAHGQVTIIKAANPCAGRKGQGSCQAALRT
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f EDLTPTTPNTEVSPRADPRLSQPEDIWPEWAWPVVVGTTMLLLLLFLAVSSLGSCSTGSP
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f APVPENDLVGIVGGHNTPGEVVVAVGADRRSLHFPEGHRPVHLPDSHQGCVSVRGPGAAE
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f CQPDRRPPNYSVFFLGADIALLKLATSSLEFTDSDNCWNTGWGMVGLLDMLPPPYRPQQV
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f KVLTLSNADCERQTYDAFPGAGDRKFIQDDMICAGRTGRRTWKGDSGGPLVCKKKGTWLQ
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f AGVVSWGFYSDRPSIGVYTRPETSWQGANHADAQRPAGRVPTMQRPRDMGQGQEWVCRPF
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f THVTCYPTAIPRPFTHVTCYLMAVPSTLTHVTCYPTAVPRPFTHVTCYLMAVPSTLTHIT
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ------------------------------------------------------------
NOV32b ------------------------------------------------------------
NOV32c ------------------------------------------------------------
NOV32d ------------------------------------------------------------
NOV32e ------------------------------------------------------------
NOV32f CYMMAVPRPFTHITCYPMAVPSTLTHVTCHPTAIPRPFTHITCYTMAIPRPSTTPPATRR
NOV32g ------------------------------------------------------------
NOV32h ------------------------------------------------------------
NOV32i ------------------------------------------------------------
NOV32j ------------------------------------------------------------
NOV32k ------------------------------------------------------------
NOV32l ------------------------------------------------------------
NOV32m ------------------------------------------------------------
NOV32n ------------------------------------------------------------
NOV32a ---------------------------
NOV32b ---------------------------
NOV32c ---------------------------
NOV32d ---------------------------
NOV32e ---------------------------
NOV32f PSPAPSPTSPATRWPSPGPSPMSPATR
NOV32g ---------------------------
NOV32h ---------------------------
NOV32i ---------------------------
NOV32j ---------------------------
NOV32k ---------------------------
NOV32l ---------------------------
NOV32m ---------------------------
NOV32n ---------------------------
NOV32a (SEQ ID NO: 382)
NOV32b (SEQ ID NO: 384)
NOV32c (SEQ ID NO: 386)
NOV32d (SEQ ID NO: 388)
NOV32e (SEQ ID NO: 390)
NOV32f (SEQ ID NO: 392)
NOV32g (SEQ ID NO: 394)
NOV32h (SEQ ID NO: 396)
NOV32i (SEQ ID NO: 398)
NOV32j (SEQ ID NO: 400)
NOV32k (SEQ ID NO: 402)
NOV32l (SEQ ID NO: 404)
NOV32m (SEQ ID NO: 406)
NOV32n (SEQ ID NO: 408)

Further analysis of the NOV32a protein yielded the following properties shown in Table 32C.

TABLE 32C
Protein Sequence Properties NOV32a

| | |
|---|---|
| SignalP analysis: | Cleavage site between residues 13 and 14 |

PSORT II analysis:
PSG: a new signal peptide prediction method

N-region: length 0; pos.chg 0; neg.chg 0
H-region: length 12; peak value 6.67
PSG score: 2.27

GvH: von Heijne's method for signal seq. recognition

GvH score (threshold: −2.1): −6.63
possible cleavage site: between 34 and 35

>>> Seems to have no N-terminal signal peptide
ALOM: Klein et al's method for TM region allocation

| Init position for calculation: 1 | | |
|---|---|---|
| Tentative number of TMS(s) for the threshold 0.5: 1 | | |
| Number of TMS(s) for threshold 0.5: 1 | | |
| INTEGRAL | Likelihood = −2.76 | Transmembrane 110-126 |

| | |
|---|---|
| PERIPHERAL | Likelihood = 1.80 (at 81) |

ALOM score: −2.76 (number of TMSs: 1)

MTOP: Prediction of membrane topology (Hartmann et al.)

Center position for calculation: 117
Charge difference: −2.0 C(0.0) − N(2.0)
N >= C: N-terminal side will be inside

>>> membrane topology: type 2 (cytoplasmic tail 1 to 110)
MITDISC: discrimination of mitochondrial targeting seq

| | | | |
|---|---|---|---|
| R content: | 0 | Hyd Moment(75): | 4.93 |
| Hyd Moment(95): | 1.50 | G content: | 5 |
| D/E content: | 2 | S/T content: | 1 |
| Score: −9.83 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 10.6%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 15
LL at 82

checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 89

COIL: Lupas's algorithm to detect coiled-coil regions

total: 0 residues

Final Results (k = 9/23):

```
34.8%: cytoplasmic
30.4%: mitochondrial
13.0%: Golgi
  8.7%: endoplasmic reticulum
  4.3%: extracellular, including cell wall
  4.3%: nuclear
  4.3%: vesicles of secretory system
```

>> prediction for CG53989-03 is cyt (k = 23)

A search of the NOV32a protein against the Geneseq database, a proprietary database that contains sequences published in patents and patent publication, yielded several homologous proteins shown in Table 32D.

TABLE 32D
Geneseq Results for NOV32a

| | | NOV32a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE14347 | Human protease PRTS-12 protein - | 130 . . . 339 | 205/210 (97%) | e−123 |
| | Homo sapiens, 262 aa. | 53 . . . 262 | 205/210 (97%) | |
| | [WO200183775-A2, 08-NOV- | | | |
| | 2001] | | | |
| AAE08591 | Human NOV12 protein - Homo | 126 . . . 339 | 205/214 (95%) | e−122 |
| | sapiens, 220 aa. [WO200161009- | 7 . . . 220 | 206/214 (95%) | |
| | A2, 23-AUG-2001] | | | |
| AAE08590 | Human NOV11 protein - Homo | 135 . . . 339 | 203/205 (99%) | e−122 |
| | sapiens, 285 aa. [WO200161009- | 81 . . . 285 | 203/205 (99%) | |
| | A2, 23-AUG-2001] | | | |
| AAU82736 | Amino acid sequence of novel | 9 . . . 334 | 237/368 (64%) | e−118 |
| | human protease #35 - Homo | 46 . . . 411 | 252/368 (68%) | |
| | sapiens, 948 aa. [WO200200860- | | | |
| | A2, 03-JAN-2002] | | | |
| AAE08587 | Human NOV8 protein - Homo | 135 . . . 339 | 198/205 (96%) | e−116 |
| | sapiens, 290 aa. [WO200161009- | 86 . . . 290 | 198/205 (96%) | |
| | A2, 23-AUG-2001] | | | |

In a BLAST search of public sequence databases, the NOV32a protein was found to have homology to the proteins shown in the BLASTP data in Table 32E.

TABLE 32E
Public BLASTP Results for NOV32a

| | | NOV32a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAP21675 | Mast cell protease-11 - Mus | 106 . . . 336 | 129/231 (55%) | 2e−60 |
| | musculus (Mouse), 318 aa. | 60 . . . 284 | 155/231 (66%) | |
| AAA30855 | Mastin precursor - Canis sp, 251 | 98 . . . 337 | 120/240 (50%) | 7e−51 |
| | aa (fragment). | 22 . . . 249 | 142/240 (59%) | |
| P19236 | Mastocytoma protease precursor | 98 . . . 337 | 120/240 (50%) | 7e−51 |
| | (EC 3.4.21.—) - Canis familiaris | 40 . . . 267 | 142/240 (59%) | |
| | (Dog), 269 aa. | | | |
| Q8SQ44 | Tryptase precursor - Sus scrofa | 143 . . . 337 | 106/195 (54%) | 1e−49 |
| | (Pig), 277 aa. | 90 . . . 275 | 128/195 (65%) | |
| Q9XSM1 | Tryptase (EC 3.4.21.59) - Ovis | 109 . . . 337 | 95/229 (41%) | 6e−43 |
| | aries (Sheep), 273 aa. | 54 . . . 273 | 129/229 (55%) | |

PFam analysis indicates that the NOV32a protein contains the domains shown in the Table 32F.

TABLE 32F
Domain Analysis of NOV32a

| | NOV32a | Identities/ | |
|---|---|---|---|
| | Match | Similarities | |
| Pfam Domain | Region | for the Matched Region | Expect Value |
| trypsin | 125 . . . 329 | 80/272 (29%) | 5.6e−32 |
| | | 156/272 (57%) | |

### Example 33

The NOV33 clone was analyzed, and the nucleotide and encoded polypeptide sequences are shown in Table 33A.

TABLE 33A
NOV33 Sequence Analysis

| | | |
|---|---|---|
| NOV33a, CG54203-02 | SEQ ID NO: 409 | 1028 bp |
| DNA Sequence | ORF Start: ATG at 18 | ORF Stop: TGA at 975 |

TGCAGAGGGGATATCACATGGAAAAAGCCAATGAGACCTCCCCTGTGATGGGGTTCGTTCTCCTGGGG
CTCTCTGCCCACCCAGAGCTGGAAAAGACATTCTTCGTGCTCATCCTGATGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33a, CG54203-02 | SEQ ID NO: 410 | 319 aa | MW at 35072.3kD |
| Protein Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIIYSLRNKDVKAAVRRLLRPKGFTQ

| | | |
|---|---|---|
| NOV33b, CG54203-01 | SEQ ID NO: 411 | 1031 bp |
| DNA Sequence | ORF Start: ATG at 22 | ORF Stop: TGA at 979 |

TGATGGCAGAGGGGATATCACATGGAAAAAGCCAATGAGACCTCCCCTGTGATGGGGTTCGTTCTCCT
GAGGCTCTCTGCCCACCCAGAGCTGGAAAAGACATTCTTCGTGCTCATCCTGCTGATGTACCTCGTGA
TCCTGCTGGGCAATGGGGTCCTCATCCTGGTGACCATCCTTGACTCCCGCCTGCACACGCCCATGTAC
TTCTTCCTAGGGAACCTCTCCTTCCTGGACATCTGCTTCACTACCTCCTCAGTCCCACTGGTCCTGGA
CAGCTTTTTGACTCCCCAGGAAACCATCTCCTTCTCAGCCTGTGCTGTGCAGATGGCACTCTCCTTTG
CCATGGCAGGAACAGAGTGCTTGCTCCTGAGCATGATGGCATTTGATCGCTATGTGGCCATCTGCAAC
CCCCTTAGGTACTCCGTGATCATGAGCAAGGCTGCCTACATGCCCATGGCTGCCAGCTCCTGGGCTAT
TGGTGGTGCTGCTTCCGTGGTACACACATCCTTGGCAATTCAGCTGCCCTTCTGTGGAGACAATGTCA
TCAACCACTTCACCTGTGAGATTCTGGCTGTTCTAAAGTTGGCCTGTGCTGACATTTCCATCAATGTG
ATCAGCATGGAGGTGACGAATGTGATCTTCCTAGGAGTCCCGGTTCTGTTCATCTCTTTCTCCTATGT
CTTCATCATCACCACCATCCTGAGGATCCCCTCAGCTGAGGGGAGGAAAAAGGTCTTCTCCACCTGCT
CTGCCCACCTCACCGTGGTGATCGTCTTCTACGGGACCTTATTCTTCATGTATGGGAAGCCTAAGTCT
AAGGACTCCATGGGAGCAGACAAAGAGGATCTTTCAGACAAACTCATCCCCCTTTTCTATGGGGTGGT
GACCCCGATGCTCAACCCCATCATCTATAGCCTGAGGAACAAGGATGTGAAGGCTGCTGTGAGGAGAC
TGCTGAGACCAAAAGGCTTCACTCAGTGATGGTGGAAGGGTCCTCTGTGATTGTCACCCACATGGAAG
TAAGGAATCAC

| | | | |
|---|---|---|---|
| NOV33b, CG54203-01 | SEQ ID NO: 412 | 319 aa | MW at 35171.4kD |
| Protein Sequence | | | |

MEKANETSPVMGFVLLRLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIIYSLRNKDVKAAVRRLLRPKGFTQ

| | | |
|---|---|---|
| NOV33c, SNP13382442 of | SEQ ID NO: 413 | 1028 bp |
| CG54203-02, DNA Sequence | ORF Start: ATG at 18 | ORF Stop: TGA at 975 |
| | SNP Pos: 66 | SNP Change: G to A |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33c, SNP13382442 of | SEQ ID NO: 414 | 319 aa | MW at 35171.4kD |
| CG54203-02, Protein | SNP Pos: 17 | | SNP Change: Gly to Arg |
| Sequence | | | |

MEKANETSPVMGFVLLRLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

| | | |
|---|---|---|
| NOV33d, SNP13373880 of | SEQ ID NO: 415 | 1028 bp |
| CG54203-02, DNA Sequence | ORF Start: ATG at 18 | ORF Stop: TGA at 975 |
| | SNP Pos: 324 | SNP Change: G to A |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGACACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33d, SNP13373880 of | SEQ ID NO: 416 | 319 aa | MW at 35102.3kD |
| CG54203-02, Protein | SNP Pos: 103 | | SNP Change: Ala to Thr |
| Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMTLSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

| | | |
|---|---|---|
| NOV33e, SNP13373881 of | SEQ ID NO: 417 | 1028 bp |
| CG54203-02, DNA Sequence | ORF Start: ATG at 18 | ORF Stop: TGA at 975 |
| | SNP Pos: 429 | SNP Change: A to G |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGGGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33e, SNP13373881 of | SEQ ID NO: 418 | 319 aa | MW at 35042.3kD |
| CG54203-02, Protein | SNP Pos: 138 | | SNP Change: Ser to Gly |
| Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MGKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

| | | |
|---|---|---|
| NOV33f, SNP13382443 of | SEQ ID NO: 419 | 1028 bp |
| CG54203-02, DNA Sequence | ORF Start: ATG at 18 | ORF Stop: TGA at 975 |
| | SNP Pos: 444 | SNP Change: A to G |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACGTGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33f, SNP13382443 of | SEQ ID NO: 420 | 319 aa | MW at 35040.2kD |
| CG54203-02, Protein | SNP Pos: 143 | | SNP Change: Met to Val |
| Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYVPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

| | | |
|---|---|---|
| NOV33g, SNP13373844 of | SEQ ID NO: 421 | 1028 bp |
| CG54203-02, DNA Sequence | ORF Start: ATG at 18 | ORF Stop: TGA at 975 |
| | SNP Pos: 719 | SNP Change: G to A |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGAAGGAAAAAGGTCTTCTCCACCTGCTCTGC
CCACCTCACCGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33g, SNP13373844 of | SEQ ID NO: 422 | 319 aa | MW at 35072.3kD |
| CG54203-02, Protein | SNP Pos: 234 | | SNP Change: Gly to Gly |
| Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

| | | |
|---|---|---|
| NOV33h, SNP13373883 of | SEQ ID NO: 423 | 1028 bp |
| CG54203-02, DNA Sequence | ORF Start: ATG at 18 | ORF Stop: TGA at 975 |
| | SNP Pos: 758 | SNP Change: C to T |

TGCAGAGGGGATATCACATGGAAAAAGCCAATGAGACCTCCCCTGTGATGGGGTTCGTTCTCCTGAGG
CTCTCTGCCCACCCAGAGCTGGAAAAGACATTCTTCGTGCTCATCCTGCTGATGTACCTCGTGATCCT
GCTGGGCAATGGGGTCCTCATCCTGGTGACCATCCTTGACTCCCGCCTGCACACGCCCATGTACTTCT
TCCTAGGGAACCTCTCCTTCCTGGACATCTGCTTCACTACCTCCTCAGTCCCACTGGTCCTGGACAGC
TTTTTGACTCCCCAGGAAACCATCTCCTTCTCAGCCTGTGCTGTGCAGATGGCACTCTCCTTTGCCAT
GGCAGGAACAGAGTGCTTGCTCCTGAGCATGATGGCATTTGATCGCTATGTGGCCATCTGCAACCCCC
TTAGGTACTCCGTGATCATGAGCAAGGCTGCCTACATGCCCATGGCTGCCAGCTCCTGGGCTATTGGT
GGTGCTGCTTCCGTGGTACACACATCCTTGGCAATTCAGCTGCCCTTCTGTGGAGACAATGTCATCAA
CCACTTCACCTGTGAGATTCTGGCTGTTCTAAAGTTGGCCTGTGCTGACATTTCCATCAATGTGATCA
GCATGGAGGTGACGAATGTGATCTTCCTAGGAGTCCCGGTTCTGTTCATCTCTTTCTCCTATGTCTTC
ATCATCACCACCATCCTGAGGATCCCCTCAGCTGAGGGGAGGAAAAAGGTCTTCTCCACCTGCTCTGC
CCACCTCACTGTGGTGATCGTCTTCTACGGGACCTTATTCTTCATGTATGGGAAGCCTAAGTCTAAGG
ACTCCATGGGAGCAGACAAAGAGGATCTTTCAGACAAACTCATCCCCCTTTTCTATGGGGTGGTGACC
CCGATGCTCAACCCCATCATCTATAGCCTGAGGAACAAGGATGTGAAGGCTGCTGTGAGGAGACTGCT
GAGACCAAAAGGCTTCACTCAGTGATGGTGGAAGGGTCCTCTGTGATTGTCACCCACATGGAAGTAAG
GAATCACA

| | | | |
|---|---|---|---|
| NOV33h, SNP13373883 of | SEQ ID NO: 424 | 319 aa | MW at 35072.3kD |
| CG54203-02, Protein | SNP Pos: 247 | | SNP Change: Thr to Thr |
| Sequence | | | |

MEKANETSPVMGFVLLGLSAHPELEKTFFVLILLMYLVILLGNGVLILVTILDSRLHTPMYFFLGNLS
FLDICFTTSSVPLVLDSFLTPQETISFSACAVQMALSFAMAGTECLLLSMMAFDRYVAICNPLRYSVI
MSKAAYMPMAASSWAIGGAASVVHTSLAIQLPFCGDNVINHFTCEILAVLKLACADISINVISMEVTN
VIFLGVPVLFISFSYVFIITTILRIPSAEGRKKVFSTCSAHLTVVIVFYGTLFFMYGKPKSKDSMGAD
KEDLSDKLIPLFYGVVTPMLNPIEYSLRNKDVKAAVRRLLRPKGFTQ

A ClustalW comparison of the above protein sequences yields the following sequence alignment shown in Table 33B.

TABLE 33B
Comparison of the NOV33 protein sequences.

NOV33a MEKANETSPVMGFVLLGLSAHPELEKTFFVLILLMYLVILLGNGVLILVTILDSRLHTPM
NOV33b MEKANETSPVMGFVLLRLSAHPELEKTFFVLILLMYLVILLGNGVLILVTILDSRLHTPM
NOV33a YFFLGNLSFLDICFTTSSVPLVLDSFLTPQETISFSACAVQMALSFAMAGTECLLLSMMA
NOV33b YFFLGNLSFLDICFTTSSVPLVLDSFLTPQETISFSACAVQMALSFAMAGTECLLLSMMA
NOV33a FDRYVAICNPLRYSVIMSKAAYMPMAASSWAIGGAASVVHTSLAIQLPFCGDNVINHFTC
NOV33b FDRYVAICNPLRYSVIMSKAAYMPMAASSWAIGGAASVVHTSLAIOLPFCGDNVINHFTC
NOV33a EILAVLKLACADISINVISMEVTNVIFLGVPVLFISFSYVFIITTILRIPSAEGRKKVFS
NOV33b EILAVLKLACADISINVISMEVTNVIFLGVPVLFISFSYVFIITTILRIPSAEGRKKVFS
NOV33a TCSAHLTVVIVFYGTLFFMYGKPKSKDSMGADKEDLSDKLIPLFYGVVTPMLNPIIYSLR
NOV33b TCSAHLTVVIVFYGTLFFMYGKPKSKDSMGADKEDLSDKLIPLFYGVVTPMLNPIIYSLR
NOV33a NKDVKAAVRRLLRPKGFTQ
NOV33b NKDVKAAVRRLLRPKGFTQ
NOV33a (SEQ ID NO: 410)
NOV33b (SEQ ID NO: 412)

Further analysis of the NOV33a protein yielded the following properties shown in Table 33C.

TABLE 33C
Protein Sequence Properties NOV33a

| | |
|---|---|
| SignalP analysis: | Cleavage site between residues 45 and 46 |

PSORT II analysis:
PSG: a new signal peptide prediction method

N-region: length 6; pos.chg 1; neg.chg 2
H-region: length 16; peak value 0.00
PSG score: −4.40

GvH: von Heijne's method for signal seq. recognition

GvH score (threshold: −2.1): −3.59
possible cleavage site: between 44 and 45

>>> Seems to have no N-terminal signal peptide
ALOM: Klein et al's method for TM region allocation

| Init position for calculation: 1 | | |
|---|---|---|
| Tentative number of TMS(s) for the threshold 0.5: 7 | | |
| INTEGRAL | Likelihood = −9.50 | Transmembrane 31-47 |
| INTEGRAL | Likelihood = −0.22 | Transmembrane 67-83 |
| INTEGRAL | Likelihood = −0.32 | Transmembrane 93-109 |
| INTEGRAL | Likelihood = −3.77 | Transmembrane 182-198 |
| INTEGRAL | Likelihood = −6.95 | Transmembrane 206-222 |
| INTEGRAL | Likelihood = −1.81 | Transmembrane 242-258 |
| INTEGRAL | Likelihood = −0.22 | Transmembrane 280-296 |

| | |
|---|---|
| PERIPHERAL | Likelihood = 2.38 (at 151) |

ALOM score: −9.50 (number of TMSs: 7)

MTOP: Prediction of membrane topology (Hartmann et al.)

Center position for calculation: 38
Charge difference: 1.0 C(0.5) − N(−0.5)
C > N: C-terminal side will be inside

>>> membrane topology: type 3b
MITDISC: discrimination of mitochondrial targeting seq

| | | | |
|---|---|---|---|
| R content: | 0 | Hyd Moment(75): | 6.12 |
| Hyd Moment(95): | 11.03 | G content: | 0 |
| D/E content: | 2 | S/T content: | 0 |
| Score: −6.47 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 7.2%
NLS Score: −0.47

KDEL: ER retention motif in the C-terminus: none
ER Membrane Retention Signals:

KKXX-like motif in the C-terminus: KGFT

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

55.6%: endoplasmic reticulum
11.1%: mitochondrial
11.1%: vacuolar
11.1%: vesicles of secretory system
11.1%: Golgi

>> prediction for CG54203-02 is end (k = 9)

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85170 | G-coupled olfactory receptor #31 - | 1 . . . 319 | 318/319 (99%) | e−179 |
| | Homo sapiens, 319 aa. | 1 . . . 319 | 318/319 (99%) | |
| | [WO200198526-A2, 27-DEC- | | | |
| | 2001] | | | |
| AAU97924 | Novel odourant receptor NOV3 | 1 . . . 319 | 318/319 (99%) | e−179 |
| | protein - Unidentified, 319 aa. | 1 . . . 319 | 318/319 (99%) | |
| | [WO200236632-A2, 10-MAY- | | | |
| | 2002] | | | |
| AAG71989 | Human olfactory receptor | 1 . . . 319 | 318/319 (99%) | e−179 |
| | polypeptide, SEQ ID NO: 1670 - | 1 . . . 319 | 318/319 (99%) | |
| | Homo sapiens, 319 aa. | | | |
| | [WO200127158-A2, 19-APR- | | | |
| | 2001] | | | |
| AAU07096 | Human odorant receptor (OR) | 1 . . . 319 | 318/319 (99%) | e−179 |
| | polypeptide #13 - Homo sapiens, | 1 . . . 319 | 318/319 (99%) | |
| | 319 aa. [WO200157215-A2, 09- | | | |
| | AUG-2001] | | | |
| AAU07086 | Human odorant receptor (OR) | 1 . . . 319 | 318/319 (99%) | e−179 |
| | polypeptide #3 - Homo sapiens, | 1 . . . 319 | 318/319 (99%) | |
| | 319 aa. [WO200157215-A2, 09- | | | |
| | AUG-2001] | | | |

In a BLAST search of public sequence databases, the NOV33a protein was found to have homology to the proteins shown in the BLASTP data in Table 33E.

TABLE 33E
Public BLASTP Results for NOV33a

| | | NOV33a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q9NQN1 | Olfactory receptor 2S2 - Homo | 1 . . . 319 | 318/319 (99%) | e−179 |
| | sapiens (Human), 319 aa. | 1 . . . 319 | 318/319 (99%) | |
| CAC88321 | Sequence 3 from Patent | 1 . . . 317 | 263/317 (82%) | e−151 |
| | WO0164879 - Homo sapiens | 1 . . . 317 | 289/317 (90%) | |
| | (Human), 318 aa. | | | |
| Q9QZ22 | Olfactory receptor | 1 . . . 319 | 263/319 (82%) | e−150 |
| | GA_x5J8B7W5BNN-979337- | 1 . . . 319 | 292/319 (91%) | |
| | 980296 - Mus musculus | | | |
| | (Mouse), 319 aa. | | | |
| CAC88320 | Sequence 1 from Patent | 1 . . . 317 | 263/318 (82%) | e−149 |
| | WO0164879 - Homo sapiens | 1 . . . 318 | 288/318 (89%) | |
| | (Human), 319 aa. | | | |
| Q9QZ19 | Olfactory receptor (Olfactory | 1 . . . 317 | 257/317 (81%) | e−147 |
| | receptor MOR262-5) - Mus | 1 . . . 317 | 283/317 (89%) | |
| | musculus (Mouse), 319 aa. | | | |

PFam analysis indicates that the NOV33a protein contains the domains shown in the Table 33F.

TABLE 33F
Domain Analysis of NOV33a

| | NOV33a | Identities/ | |
|---|---|---|---|
| | Match | Similarities | |
| Pfam Domain | Region | for the Matched Region | Expect Value |
| 7tm_1 | 42 . . . 297 | 57/280 (20%) | 3.7e−35 |
| | | 179/280 (64%) | |

### Example 34

The NOV34 clone was analyzed, and the nucleotide and encoded polypeptide sequences are shown in Table 34A.

TABLE 34A
NOV34 Sequence Analysis

| | | |
|---|---|---|
| NOV34a, CG54212-02 | SEQ ID NO: 425 | 953 bp |
| DNA Sequence | ORF Start: ATG at 11 | ORF Stop: TGA at 947 |

ACTCTGCAGCATGGAGCCGCTCAACAGAACAGAGGTGTCCGAGTTCTTTCTGAAAGGATTTTCTGGCT
ACCCAGCCCTGGAGCATCTGCTCTTCCCTCTGTGCTCAGCCATGTACCTGGTGACCCTCCTGGGGAAC
ACAGCCATCATGGCGGTGAGCGTGCTAGATATCCACCTGCACACGCCTGTGTACTTCTTCCTGGGCAA
CCTCTCTACCCTGGACATCTGCTACACGCCCACCTTTGTGCCTCTGATGCTGGTCCACCTCCTGTCAT
CCCGGAAGACCATCTCCTTTGCTGTCTGTGCCATCCAGATGTGTCTGAGCCTGTCCACGGGCTCCACG
GAGTGCCTGCTACTGGCCATCACGGCCTATGACCGCTACCTGGCCATCTGCCAGCCACTCAGGTACCG
CGTGCTCATGAGCCACCGGCTCTGCGTGCTGCTGATGGGAGCTGCCTGGGTCCTCTGCCTCCTCAAGT
CGGTGACTGAGATGGTCATCTCCATGAGGCTGCCCTTCTGTGGCCACCACGTGGTCAGTCACTTCACC
TGCAAGATCCTGGCAGTGCTGAAGCTGGCATGCGGCAACACGTCGGTCAGCGAAGACTTCCTGCTGGC
GGGCTCCATCCTGCTGCTGCCTGTACCCCTGGCATTCATCTGCCTGTCCTACTTGCTCATCCTGGCCA
CCATCCTGAGGGTGCCCTCGGCCGCCAGGTGCTGCAAAGCCTTCTCCACCTGCTTGGCACACCTGGCT
GTAGTGCTGCTTTTCTACGGCACCATCATCTTCATGTACTTGAAGCCCAAGAGTAAGGAAGCCCACAT
CTCTGATGAGGTCTTCACAGTCCTCTATGCCATGGTCACGACCATGCTGAACCCCACCATCTACAGCC
TGAGGAACAAGGAGGTGAAGGAGGCCGCCAGGAAGGTGTGGGGCAGGAGTCGGGCCTCCAGGTGAGGG
A

| | | | |
|---|---|---|---|
| NOV34a, GG54212-02 | SEQ ID NO: 426 | 312 aa | MW at 34707.2kD |
| Protein Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34b, GG54212-01 | SEQ ID NO: 427 | 1050 bp |
| DNA Sequence | ORF Start: ATG at 59 | ORF Stop: TGA at 995 |

CCCTGTACCCTCTCTCCTTCCATCCCAGCTGTGGACCATCTCTTCAGAACTCTGCAGCATGGAGCCGC
TCAACAGAACAGAGGTGTCCGAGTTCTTTCTGAAAGGATTTTCTGGCTACCCAGCCCTGGAGCATCTG
CTCTTCCCTCTGTGCTCAGCCATGTACCTGGTGACCCTCCTGGGGAACACAGCCATCATGGCGGTGAG
CGTGCTAGATATCCACCTGCACACGCCCGTGTACTTCTTCCTGGGCAACCTCTCTACCCTGGACATCT
GCTACACGCCCACCTTTGTGCCTCTGATGCTGGTCCACCTCCTGTCATCCCGGAAGACCATCTCCTTT
GCTGTCTGTGCCATCCAGATGTGTCTGAGCCTGTCCACGGGCTCCACGGAGTGCCTGCTACTGGCCAT
CACGGCCTATGACCGCTACCTGGCCATCTGCCAGCCACTCAGGTACCACGTGCTCATGAGCCACCGGC
TCTGCGTGCTGCTGATGGGAGCTGCCTGGGTCCTCTGCCTCCTCAAGTCGGTGACTGAGATGGTCATC
TCCATGAGGCTGCCCTTCTGTGGCCACCACGTGGTCAGTCACTTCACCTGCAAGATCCTGGCAGTGCT
GAAGCTGGCATGCGGCAACACGTCGGTCAGCGAAGACTTCCTGCTGGCGGGCTCCATCCTGCTGCTGC
CTGTACCCCTGGCATTCATCTGCCTGTCCTACTTGCTCATCCTGGCCACCATCCTGAGGGTGCCCTCG
GCCGCCAGGTGCTGCAAAGCCTTCTCCACCTGCTTGGCACACCTGGCTGTAGTGCTGCTTTTCTACGG
CACCATCATCTTCATGTACTTGAAGCCCAAGAGTAAGGAAGCCCACATCTCTGATGAGGTCTTCACAG
TCCTCTATGCCATGGTCACGACCATGCTGAACCCCACCATCTACAGCCTGAGGAACAAGGAGGTGAAG
GAGGCCGCCAGGAAGGTGTGGGGCAGGAGTCGGGCCTCCAGGTGAGGGAGGGCGGGGCTCTGTACAGA
CGCAGGTCTCAGGTTAGTAGCTGAGGCCAT

| | | | |
|---|---|---|---|
| NOV34b, CG54212-01 | SEQ ID NO: 428 | 312 aa | MW at 34688.2kD |
| Protein Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34c, CG54212-03 | SEQ ID NO: 429 | 917 bp |
| DNA Sequence | ORF Start: at 3 | ORF Stop: TGA at 861 |

TGCTCTTCCCTCTGTGCTCAGCCATGTACCTGGTGACCCTCCTGGGGAACACAGCCATCATGGCGGTG
AGCGTGCTAGATATCCACCTGCACACGCCCGTGTACTTCTTCCTGGGCAACCTCTCTACCCTGGACAT
CTGCTACACGCCCACCTTTGTGCCTCTGATGCTGGTCCACCTCCTGTCATCCCGGAAGACCATCTCCT
TTGCTGTCTGTGCCATCCAGATGTGTCTGAGCCTGTCCACGGGCTCCACGGAGTGCCTGCTACTGGCC
ATCACGGCCTATGACCGCTACCTGGCCATCTGCCAGCCACTCAGGTACCACGTGCTCATGAGCCACCG
GCTCTGCGTGCTGCTGATGGGAGCTGCCTGGGTCCTCTGCCTCCTCAAGTCGGTGACTGAGATGGTCA
TCTCCATGAGGCTGCCCTTCTGTGGCCACCACGTGGTCAGTCACTTCACCTGCAAGATCCTGGCAGTG
CTGAAGCTGGCATGCGGCAACACGTCGGTCAGCGAAGACTTCCTGCTGGCGGGCTCCATCCTGCTGCT
GCCTGTACCCCTGGCATTCATCTGCCTGTCCTACTTGCTCATCCTGGCCACCATCCTGAGGGTGCCCT
CGGCCGCCAGGTGCTGCAAAGCCTTCTCCACCTGCTTGGCACACCTGGCTGTAGTGCTGCTTTTCTAC
GGCACCATCATCTTCATGTACTTGAAGCCCAAGAGTAAGGAAGCCCACATCTCTGATGAGGTCTTCAC
AGTCCTCTATGCCATGGTCACGACCATGCTGAACCCCACCATCTACAGCCTGAGGAACAAGGAGGTGA
AGGAGGCCGCCAGGAAGGTGTGGGGCAGGAGTCGGGCCTCCAGGTGAGGGAGGGCGGGGCTCTGTACA
GACGCAGGTCTCAGGTTAGTAGCTGAGGCCATC

| | | |
|---|---|---|
| NOV34c, CG54212-03 | SEQ ID NO: 430 | 286 aa MW at 31693.8kD |
| Protein Sequence | | |

LFPLCSAMYLVTLLGNTAIMAVSVLDIHLHTPVYFFLGNLSTLDICYTPTFVPLMLVHLLSSRKTISF
AVCAIQMCLSLSTGSTECLLLAITAYDRYLAICQPLRYHVLMSHRLCVLLMGAAWVLCLLKSVTEMVI
SMRLPFCGHHVVSHFTCKILAVLKLACGNTSVSEDFLLAGSILLLPVPLAFICLSYLLILATILRVPS
AARCCKAFSTCLAHLAVVLLFYGTIIFMYLKPKSKEAHISDEVFTVLYAMVTTMLNPTIYSLRNKEVK
EAARKVWGRSRASR

| | | |
|---|---|---|
| NOV34d, CG54212-04 | SEQ ID NO: 431 | 1025 bp |
| DNA Sequence | ORF Start: ATG at 33 | ORF Stop: TGA at 969 |

AGCTGTGGACCATCTCTTCAGAACTCTGCAGCATGGAGCCGCTCAACAGAACAGAGGTGTCCGAGTTC
TTTCTGAAAGGATTTTCTGGCTACCCAGCCCTGGAGCATCTGCTCTTCCCTCTGTGCTCAGCCATGTA
CCTGGTGACCCTCCTGGGGAACACAGCCATCATGGCGGTGAGCGTGCTAGATATCCACCTGCACACGC
CCGTGTACTTCTTCCTGGGCAACCTCTCTACCCTGGACATCTGCTACACGCCCACCTTTGTGCCTCTG
ATGCTGGTCCACCTCCTGTCATCCCGGAAGACCATCTCCTTTGCTGTCTGTGCCATCCAGATGTGTCT
GAGCCTGTCCACGGGCTCCACGGAGTGCCTGCTACTGGCCATCACGGCCTATGACCGCTACCTGGCCA
TCTGCCAGCCACTCAGGTACCACGTGCTCATGAGCCACCGGCTCTGCGTGCTGCTGATGGGAGCTGCC
TGGGTCCTCTGCCTCCTCAAGTCGGTGACTGAGATGGTCATCTCCATGAGGCTGCCCTTCTGTGGCCA
CCACGTGGTCAGTCACTTCACCTGCAAGATCCTGGCAGTGCTGAAGCTGGCATGCGGCAACACGTCGG
TCAGCGAAGACTTCCTGCTGGCGGGCTCCATCCTGCTGCTGCCTGTACCCCTGGCATTCATCTGCCTG
TCCTACTTGCTCATCCTGGCCACCATCCTGAGGGTGCCCTCGGCCGCCAGGTGCTGCAAAGCCTTCTC
CACCTGCTTGGCACACCTGGCTGTAGTGCTGCTTTTCTACGGCACCATCATCTTCATGTACTTGAAGC
CCAAGAGTAAGGAAGCCCACATCTCTGATGAGGTCTTCACAGTCCTCTATGCCATGGTCACGACCATG
CTGAACCCCACCATCTACAGCCTGAGGAACAAGGAGGTGAAGGAGGCCGCCAGGAAGGTGTGGGGCAG
GAGTCGGGCCTCCAGGTGAGGGAGGGCGGGGCTCTGTACAGACGCAGGTCTCAGGTTAGTAGCTGAGG
CCATC

| | | | |
|---|---|---|---|
| NOV34d, CG54212-04 | SEQ ID NO: 432 | 312 aa | MW at 34688.2kD |
| Protein Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34e, SNP13373981 of | SEQ ID NO: 433 | 953 bp |
| CG54212-02, DNA Sequence | ORF Start: ATG at 11 | ORF Stop: TGA at 947 |
| | SNP Pos: 184 | SNP Change: T to C |

ACTCTGCAGCATGGAGCCGCTCAACAGAACAGAGGTGTCCGAGTTCTTTCTGAAAGGATTTTCTGGCT
ACCCAGCCCTGGAGCATCTGCTCTTCCCTCTGTGCTCAGCCATGTACCTGGTGACCCTCCTGGGGAAC
ACAGCCATCATGGCGGTGAGCGTGCTAGATATCCACCTGCACACGCCCGTGTACTTCTTCCTGGGCAA
CCTCTCTACCCTGGACATCTGCTACACGCCCACCTTTGTGCCTCTGATGCTGGTCCACCTCCTGTCAT
CCCGGAAGACCATCTCCTTTGCTGTCTGTGCCATCCAGATGTGTCTGAGCCTGTCCACGGGCTCCACG
GAGTGCCTGCTACTGGCCATCACGGCCTATGACCGCTACCTGGCCATCTGCCAGCCACTCAGGTACCG
CGTGCTCATGAGCCACCGGCTCTGCGTGCTGCTGATGGGAGCTGCCTGGGTCCTCTGCCTCCTCAAGT
CGGTGACTGAGATGGTCATCTCCATGAGGCTGCCCTTCTGTGGCCACCACGTGGTCAGTCACTTCACC
TGCAAGATCCTGGCAGTGCTGAAGCTGGCATGCGGCAACACGTCGGTCAGCGAAGACTTCCTGCTGGC
GGGCTCCATCCTGCTGCTGCCTGTACCCCTGGCATTCATCTGCCTGTCCTACTTGCTCATCCTGGCCA
CCATCCTGAGGGTGCCCTCGGCCGCCAGGTGCTGCAAAGCCTTCTCCACCTGCTTGGCACACCTGGCT
GTAGTGCTGCTTTTCTACGGCACCATCATCTTCATGTACTTGAAGCCCAAGAGTAAGGAAGCCCACAT
CTCTGATGAGGTCTTCACAGTCCTCTATGCCATGGTCACGACCATGCTGAACCCCACCATCTACAGCC
TGAGGAACAAGGAGGTGAAGGAGGCCGCCAGGAAGGTGTGGGGCAGGAGTCGGGCCTCCAGGTGAGGG
A

| | | | |
|---|---|---|---|
| NOV34e, SNP13373981 of | SEQ ID NO: 434 | 312 aa | MW at 34707.2kD |
| CG54212-02, Protein | SNP Pos: 58 | | SNP Change: Pro to Pro |
| Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34f, SNP13373982 of | SEQ ID NO: 435 | 953 bp |
| CG54212-02, DNA Sequence | ORF Start: ATG at 11 | ORF Stop: TGA at 947 |
| | SNP Pos: 188 | SNP Change: T to C |

ACTCTGCAGCATGGAGCCGCTCAACAGAACAGAGGTGTCCGAGTTCTTTCTGAAAGGATTTTCTGGCT
ACCCAGCCCTGGAGCATCTGCTCTTCCCTCTGTGCTCAGCCATGTACCTGGTGACCCTCCTGGGGAAC
ACAGCCATCATGGCGGTGAGCGTGCTAGATATCCACCTGCACACGCCTGTGCACTTCTTCCTGGGCAA
CCTCTCTACCCTGGACATCTGCTACACGCCCACCTTTGTGCCTCTGATGCTGGTCCACCTCCTGTCAT
CCCGGAAGACCATCTCCTTTGCTGTCTGTGCCATCCAGATGTGTCTGAGCCTGTCCACGGGCTCCACG
GAGTGCCTGCTACTGGCCATCACGGCCTATGACCGCTACCTGGCCATCTGCCAGCCACTCAGGTACCG
CGTGCTCATGAGCCACCGGCTCTGCGTGCTGCTGATGGGAGCTGCCTGGGTCCTCTGCCTCCTCAAGT
CGGTGACTGAGATGGTCATCTCCATGAGGCTGCCCTTCTGTGGCCACCACGTGGTCAGTCACTTCACC
TGCAAGATCCTGGCAGTGCTGAAGCTGGCATGCGGCAACACGTCGGTCAGCGAAGACTTCCTGCTGGC
GGGCTCCATCCTGCTGCTGCCTGTACCCCTGGCATTCATCTGCCTGTCCTACTTGCTCATCCTGGCCA
CCATCCTGAGGGTGCCCTCGGCCGCCAGGTGCTGCAAAGCCTTCTCCACCTGCTTGGCACACCTGGCT
GTAGTGCTGCTTTTCTACGGCACCATCATCTTCATGTACTTGAAGCCCAAGAGTAAGGAAGCCCACAT
CTCTGATGAGGTCTTCACAGTCCTCTATGCCATGGTCACGACCATGCTGAACCCCACCATCTACAGCC
TGAGGAACAAGGAGGTGAAGGAGGCCGCCAGGAAGGTGTGGGGCAGGAGTCGGGCCTCCAGGTGAGGG
A

| | | | |
|---|---|---|---|
| NOV34f, SNP13373982 of | SEQ ID NO: 436 | 312 aa | MW at 34681.2kD |
| CG54212-02, Protein | SNP Pos: 60 | | SNP Change: Tyr to His |
| Sequence | | | |

MEPLNRTEVSEFFLKGFSGYPALEHLLFPLCSAMYLVTLLGNTAIMAVSVLDIHLHTPVHFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34g, SNP13373984 of | SEQ ID NO: 437 | 953 bp |
| CG54212-02, DNA Sequence | ORF Start: ATG at 11 | ORF Stop: TGA at 947 |
| | SNP Pos: 408 | SNP Change: G to A |

ACTCTGCAGCATGGAGCCGCTCAACAGAACAGAGGTGTCCGAGTTCTTTCTGAAAGGATTTTCTGGCT
ACCCAGCCCTGGAGCATCTGCTCTTCCCTCTGTGCTCAGCCATGTACCTGGTGACCCTCCTGGGGAAC
ACAGCCATCATGGCGGTGAGCGTGCTAGATATCCACCTGCACACGCCTGTGTACTTCTTCCTGGGCAA
CCTCTCTACCCTGGACATCTGCTACACGCCCACCTTTGTGCCTCTGATGCTGGTCCACCTCCTGTCAT
CCCGGAAGACCATCTCCTTTGCTGTCTGTGCCATCCAGATGTGTCTGAGCCTGTCCACGGGCTCCACG
GAGTGCCTGCTACTGGCCATCACGGCCTATGACCGCTACCTGGCCATCTGCCAGCCACTCAGGTACCA
CGTGCTCATGAGCCACCGGCTCTGCGTGCTGCTGATGGGAGCTGCCTGGGTCCTCTGCCTCCTCAAGT
CGGTGACTGAGATGGTCATCTCCATGAGGCTGCCCTTCTGTGGCCACCACGTGGTCAGTCACTTCACC
TGCAAGATCCTGGCAGTGCTGAAGCTGGCATGCGGCAACACGTCGGTCAGCGAAGACTTCCTGCTGGC
GGGCTCCATCCTGCTGCTGCCTGTACCCCTGGCATTCATCTGCCTGTCCTACTTGCTCATCCTGGCCA
CCATCCTGAGGGTGCCCTCGGCCGCCAGGTGCTGCAAAGCCTTCTCCACCTGCTTGGCACACCTGGCT
GTAGTGCTGCTTTTCTACGGCACCATCATCTTCATGTACTTGAAGCCCAAGAGTAAGGAAGCCCACAT
CTCTGATGAGGTCTTCACAGTCCTCTATGCCATGGTCACGACCATGCTGAACCCCACCATCTACAGCC
TGAGGAACAAGGAGGTGAAGGAGGCCGCCAGGAAGGTGTGGGGCAGGAGTCGGGCCTCCAGGTGAGGG
A

| | | | |
|---|---|---|---|
| NOV34g, SNP13373984 of | SEQ ID NO: 438 | 312 aa | MW at 34688.2kD |
| CG54212-02, Protein | SNP Pos: 133 | | SNP Change: Arg to His |
| Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYHVLM
SHRLCVLLMGAAWVLCLLKSVTEMVISMRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

| | | |
|---|---|---|
| NOV34h, SNP13373985 of | SEQ ID NO: 439 | 953 bp |
| CG54212-02, DNA Sequence | ORF Start: ATG at 11 | ORF Stop: TGA at 947 |
| | SNP Pos: 501 | SNP Change: T to C |

ACTCTGCAGCATGGAGCCGCTCAACAGAACAGAGGTGTCCGAGTTCTTTCTGAAAGGATTTTCTGGCT
ACCCAGCCCTGGAGCATCTGCTCTTCCCTCTGTGCTCAGCCATGTACCTGGTGACCCTCCTGGGGAAC
ACAGCCATCATGGCGGTGAGCGTGCTAGATATCCACCTGCACACGCCTGTGTACTTCTTCCTGGGCAA
CCTCTCTACCCTGGACATCTGCTACACGCCCACCTTTGTGCCTCTGATGCTGGTCCACCTCCTGTCAT
CCCGGAAGACCATCTCCTTTGCTGTCTGTGCCATCCAGATGTGTCTGAGCCTGTCCACGGGCTCCACG
GAGTGCCTGCTACTGGCCATCACGGCCTATGACCGCTACCTGGCCATCTGCCAGCCACTCAGGTACCG
CGTGCTCATGAGCCACCGGCTCTGCGTGCTGCTGATGGGAGCTGCCTGGGTCCTCTGCCTCCTCAAGT
CGGTGACTGAGATGGTCATCTCCACGAGGCTGCCCTTCTGTGGCCACCACGTGGTCAGTCACTTCACC
TGCAAGATCCTGGCAGTGCTGAAGCTGGCATGCGGCAACACGTCGGTCAGCGAAGACTTCCTGCTGGC
GGGCTCCATCCTGCTGCTGCCTGTACCCCTGGCATTCATCTGCCTGTCCTACTTGCTCATCCTGGCCA
CCATCCTGAGGGTGCCCTCGGCCGCCAGGTGCTGCAAAGCCTTCTCCACCTGCTTGGCACACCTGGCT
GTAGTGCTGCTTTTCTACGGCACCATCATCTTCATGTACTTGAAGCCCAAGAGTAAGGAAGCCCACAT
CTCTGATGAGGTCTTCACAGTCCTCTATGCCATGGTCACGACCATGCTGAACCCCACCATCTACAGCC
TGAGGAACAAGGAGGTGAAGGAGGCCGCCAGGAAGGTGTGGGGCAGGAGTCGGGCCTCCAGGTGAGGG
A

| | | | |
|---|---|---|---|
| NOV34h, SNP13373985 of | SEQ ID NO: 440 | 312 aa | MW at 34677.1kD |
| CG54212-02, Protein | SNP Pos: 164 | | SNP Change: Met to Thr |
| Sequence | | | |

MEPLNRTEVSEFFLKGFSGYPALEHLLFPLCSAMYLVTLLGNTAIMAVSVLDIHLHTPVYFFLGNLST
LDICYTPTFVPLMLVHLLSSRKTISFAVCAIQMCLSLSTGSTECLLLAITAYDRYLAICQPLRYRVLM
SHRLCVLLMGAAWVLCLLKSVTEMVISTRLPFCGHHVVSHFTCKILAVLKLACGNTSVSEDFLLAGSI
LLLPVPLAFICLSYLLILATILRVPSAARCCKAFSTCLAHLAVVLLFYGTIIFMYLKPKSKEAHISDE
VFTVLYAMVTTMLNPTIYSLRNKEVKEAARKVWGRSRASR

A ClustalW comparison of the above protein sequences yields the following sequence alignment shown in Table 34B.

TABLE 34B
Comparison of the NOV34 protein sequences.

NOV34a MEPLNRTEVSEFFLKGFSGYPALEHLLFPLCSAMYLVTLLGNTAIMAVSVLDIHLHTPVY
NOV34b MEPLNRTEVSEFFLKGFSGYPALEHLLFPLCSAMYLVTLLGNTAIMAVSVLDIHLHTPVY
NOV34c --------------------------LFPLCSAMYLVTLLGNTAIMAVSVLDIHLHTPVY
NOV34d MEPLNRTEVSEFFLKGFSGYPALEHLLFPLCSAMYLVTLLGNTAIMAVSVLDIHLHTPVY
NOV34a FFLGNLSTLDICYTPTFVPLMLVHLLSSRKTISFAVCAIQMCLSLSTGSTECLLLAITAY
NOV34b FFLGNLSTLDICYTPTFVPLMLVHLLSSRKTISFAVCAIQMCLSLSTGSTECLLLAITAY
NOV34c FFLGNLSTLDICYTPTFVPLMLVHLLSSRKTISFAVCAIQMCLSLSTGSTECLLLAITAY
NOV34d FFLGNLSTLDICYTPTFVPLMLVHLLSSRKTISFAVCAIQMCLSLSTGSTECLLLAITAY
NOV34a DRYLAICQPLRYRVLMSHRLCVLLMGAAWVLCLLKSVTEMVISMRLPFCGHHVVSHFTCK
NOV34b DRYLAICQPLRYHVLMSHRLCVLLMGAAWVLCLLKSVTEMVISMRLPFCGHHVVSHFTCK
NOV34c DRYLAICQPLRYHVLMSHRLCVLLMGAAWVLCLLKSVTEMVISMRLPFCGHHVVSHFTCK
NOV34d DRYLAICQPLRYHVLMSHRLCVLLMGAAWVLCLLKSVTEMVISMRLPFCGHHVVSHFTCK
NOV34a ILAVLKLACGNTSVSEDFLLAGSILLLPVPLAFICLSYLLILATILRVPSAARCCKAFST
NOV34b ILAVLKLACGNTSVSEDFLLAGSILLLPVPLAFICLSYLLILATILRVPSAARCCKAFST
NOV34c ILAVLKLACGNTSVSEDFLLAGSILLLPVPLAFICLSYLLILATILRVPSAARCCKAFST
NOV34d ILAVLKLACGNTSVSEDFLLAGSILLLPVPLAFICLSYLLILATILRVPSAARCCKAFST
NOV34a CLAHLAVVLLFYGTIIFMYLKPKSKEAHISDEVFTVLYAMVTTMLNPTIYSLRNKEVKEA
NOV34b CLAHLAVVLLFYGTIIFMYLKPKSKEAHISDEVFTVLYAMVTTMLNPTIYSLRNKEVKEA
NOV34c CLAHLAVVLLFYGTIIFMYLKPKSKEAHISDEVFTVLYAMVTTMLNPTIYSLRNKEVKEA
NOV34d CLAHLAVVLLFYGTIIFMYLKPKSKEAHISDEVFTVLYAMVTTMLNPTIYSLRNKEVKEA
NOV34a ARKVWGRSRASR
NOV34b ARKVWGRSRASR
NOV34c ARKVWGRSRASR
NOV34d ARKVWGRSRASR
NOV34a (SEQ ID NO: 426)
NOV34b (SEQ ID NO: 428)
NOV34c (SEQ ID NO: 430)
NOV34d (SEQ ID NO: 432)

Further analysis of the NOV34a protein yielded the following properties shown in Table 34C.

TABLE 34C
Protein Sequence Properties NOV34a

| | |
|---|---|
| SignalP analysis: | Cleavage site between residues 48 and 49 |

PSORT II analysis:
PSG: a new signal peptide prediction method

N-region: length 11; pos.chg 1; neg.chg 3
H-region: length 3; peak value 0.00
PSG score: −4.40

GvH: von Heijne's method for signal seq. recognition

GvH score (threshold: −2.1): −2.43
possible cleavage site: between 33 and 34

>>> Seems to have no N-terminal signal peptide
ALOM: Klein et al's method for TM region allocation

| Init position for calculation: 1 | | |
|---|---|---|
| Tentative number of TMS(s) for the threshold 0.5: 6 | | |
| INTEGRAL | Likelihood = −1.86 | Transmembrane 35-51 |
| INTEGRAL | Likelihood = −0.11 | Transmembrane 92-108 |
| INTEGRAL | Likelihood = −4.88 | Transmembrane 141-157 |
| INTEGRAL | Likelihood = −0.27 | Transmembrane 173-189 |
| INTEGRAL | Likelihood = −7.80 | Transmembrane 204-220 |
| INTEGRAL | Likelihood = −6.26 | Transmembrane 241-257 |

| | |
|---|---|
| PERIPHERAL | Likelihood = 0.79 (at 71) |

ALOM score: −7.80 (number of TMSs: 6)

MTOP: Prediction of membrane topology (Hartmann et al.)

Center position for calculation: 42
Charge difference: 0.5 C(0.0) − N(−0.5)
C > N: C-terminal side will be inside

>>> membrane topology: type 3b
MITDISC: discrimination of mitochondrial targeting seq

| | | | |
|---|---|---|---|
| R content: | 1 | Hyd Moment(75): | 6.33 |
| Hyd Moment(95): | 9.16 | G content: | 0 |
| D/E content: | 2 | S/T content: | 1 |
| Score: −5.44 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 8.3%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

55.6%: endoplasmic reticulum
11.1%: Golgi
11.1%: vacuolar
11.1%: vesicles of secretory system
11.1%: mitochondrial

>> prediction for CG54212-02 is end (k = 9)

A search of the NOV34a protein against the Geneseq database, a proprietary database that contains sequences published in patents and patent publication, yielded several homologous proteins shown in Table 34D.

TABLE 34D
Geneseq Results for NOV34a

| | | NOV34a | Identities/ | |
|---|---|---|---|---|
| | | Residues/ | Similarities | |
| Geneseq | Protein/Organism/Length | Match | the Matched | Expect |
| Identifier | [Patent #, Date] | Residues | Region | Value |
| AAU85168 | G-coupled olfactory receptor #29 - | 1 . . . 312 | 311/312 (99%) | e−178 |
| | Homo sapiens, 312 aa. | 1 . . . 312 | 311/312 (99%) | |
| | [WO200198526-A2, 27-DEC- | | | |
| | 2001] | | | |
| AAU95519 | Human olfactory and pheromone | 1 . . . 312 | 311/312 (99%) | e−178 |
| | G protein-coupled receptor #6 - | 1 . . . 312 | 311/312 (99%) | |
| | Homo sapiens, 312 aa. | | | |
| | [WO200224726-A2, 28-MAR- | | | |
| | 2002] | | | |
| AAU97925 | Novel odourant receptor NOV4 | 1 . . . 312 | 311/312 (99%) | e−178 |
| | protein - Unidentified, 312 aa. | 1 . . . 312 | 311/312 (99%) | |
| | [WO200236632-A2, 10-MAY- | | | |
| | 2002] | | | |
| AAU97923 | Novel odourant receptor NOV2 | 1 . . . 312 | 311/312 (99%) | e−178 |
| | protein - Unidentified, 312 aa. | 1 . . . 312 | 311/312 (99%) | |
| | [WO200236632-A2, 10-MAY- | | | |
| | 2002] | | | |
| AAU97922 | Novel odourant receptor NOV1 | 1 . . . 312 | 311/312 (99%) | e−178 |
| | protein - Unidentified, 312 aa. | 1 . . . 312 | 311/312 (99%) | |
| | [WO200236632-A2, 10-MAY- | | | |
| | 2002] | | | |

In a BLAST search of public sequence databases, the NOV34a protein was found to have homology to the proteins shown in the BLASTP data in Table 34E.

TABLE 34E
Public BLASTP Results for NOV34a

| | | NOV34a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8NGT2 | Seven transmembrane helix | 1 . . . 312 | 311/312 (99%) | e−178 |
| | receptor - Homo sapiens | 1 . . . 312 | 311/312 (99%) | |
| | (Human), 312 aa. | | | |
| CAD57893 | Sequence 9 from Patent | 1 . . . 312 | 310/312 (99%) | e−177 |
| | WO0236632 - Homo sapiens | 1 . . . 312 | 311/312 (99%) | |
| | (Human), 312 aa. | | | |
| Q9QZ18 | Olfactory receptor (Olfactory | 1 . . . 312 | 256/312 (82%) | e−146 |
| | receptor MOR262-4) - Mus | 1 . . . 312 | 275/312 (88%) | |
| | musculus (Mouse), 312 aa. | | | |
| Q96R40 | Olfactory receptor - Homo | 68 . . . 283 | 215/216 (99%) | e−120 |
| | sapiens (Human), 216 aa | 1 . . . 216 | 215/216 (99%) | |
| | (fragment). | | | |
| CAD42439 | Sequence 121 from Patent | 1 . . . 307 | 167/307 (54%) | 5e−94 |
| | WO0212343 - Homo sapiens | 1 . . . 305 | 225/307 (72%) | |
| | (Human), 311 aa. | | | |

PFam analysis indicates that the NOV34a protein contains the domains shown in the Table 34F.

TABLE 34F
Domain Analysis of NOV34a

| | NOV34a | Identities/ | |
|---|---|---|---|
| Pfam | Match | Similarities | |
| Domain | Region | for the Matched Region | Expect Value |
| 7tm_1 | 41 . . . 290 | 63/276 (23%) | 2.8e−34 |
| | | 182/276 (66%) | |

### Example 35

The NOV35 clone was analyzed, and the nucleotide and encoded polypeptide sequences are shown in Table 35A.

TABLE 35A
NOV35 Sequence Analysis

| | | |
|---|---|---|
| NOV35a, CG54236-01 | SEQ ID NO: 441 | 1260 bp |
| DNA Sequence | ORF Start: ATG at 105 | ORF Stop: TAA at 1143 |

TGCTCCCTGTTTCATTAAAACCTAGAGAGATGTAATCAGTAAGCAAGAAGGAAAAAGGGAAATTCACA
AAGTAACTTTTTGTGTCTGTTTCTTTTTAACCCAGCATGGAGAGAAAATTTATGTCCTTGCAACCATC
CATCTCCGTATCAGAAATGGAACCAAATGGCACCTTCAGCAATAACAACAGCAGGAACTGCACAATTG
AAAACTTCAAGAGAGAATTTTTCCCAATTGTATATCTGATAATATTTTTCTGGGGAGTCTTGGGAAAT
GGGTTGTCCATATATGTTTTCCTGCAGCCTTATAAGAAGTCCACATCTGTGAACGTTTTCATGCTAAA
TCTGGCCATTTCAGATCTCCTGTTCATAAGCACGCTTCCCTTCAGGGCTGACTATTATCTTAGAGGCT
CCAATTGGATATTTGGAGACCTGGCCTGCAGGATTATGTCTTATTCCTTGTATGTCAACATGTACAGC
AGTATTTATTTCCTGACCGTGCTGAGTGTTGTGCGTTTCCTGGCAATGGTTCACCCCTTTCGGCTTCT
GCATGTCACCAGCATCAGGAGTGCCTGGATCCTCTGTGGGATCATATGGATCCTTATCATGGCTTCCT
CAATAATGCTCCTGGACAGTGGCTCTGAGCAGAACGGCAGTGTCACATCATGCTTAGAGCTGAATCTC
TATAAAATTGCTAAGCTGCAGACCATGAACTATATTGCCTTGGTGGTGGGCTGCCTGCTGCCATTTTT
CACACTCAGCATCTGTTATCTGCTGATCATTCGGGTTCTGTTAAAAGTGGAGGTCCCAGAATCGGGGC
TGCGGGTTTCTCACAGGAAGGCACTGACCACCATCATCATCACCTTGATCATCTTCTTCTTGTGTTTC
CTGCCCTATCACACACTGAGGACCGTCCACTTGACGACATGGAAAGTGGGTTTATGCAAAGACAGACT
GCATAAAGCTTTGGTTATCACACTGGCCTTGGCAGCAGCCAATGCCTGCTTCAATCCTCTGCTCTATT
ACTTTGCTGGGGAGAATTTTAAGGACAGACTAAAGTCTGCACTCAGAAAAGGCCATCCACAGAAGGCA
AAGACAAAGTGTGTTTTCCCTGTTAGTGTGTGGTTGAGAAAGGAAACAAGAGTATAAGGAGCTCTTAG
ATGAGACCTGTTCTTGTATCCTTGTGTCCATCTTCATTCACTCATAGTCTCCAAATGACTTTGTATTT
ACATCACTCCCAACAAATGTTGATTCTTAATATTTA

| | | | |
|---|---|---|---|
| NOV35a, CG54236-01 | SEQ ID NO: 442 | 346 aa | MW at 39634.7kD |
| Protein Sequence | | | |

MERKFMSLQPSISVSEMEPNGTFSNNNSRNCTIENFKREFFPIVYLIIFFWGVLGNGLSIYVFLQPYK
KSTSVNVFMLNLAISDLLFISTLPFRADYYLRGSNWIFGDLACRIMSYSLYVNMYSSIYFLTVLSVVR
FLAMVHPFRLLHVTSIRSAWILCGIIWILIMASSIMLLDSGSEQNGSVTSCLELNLYKIAKLQTMNYI
ALVVGCLLPFFTLSICYLLIIRVLLKVEVPESGLRVSHRKALTTIIITLIIFFLCFLPYHTLRTVHLT
TWKVGLCKDRLHKALVITLALAAANACFNPLLYYFAGENFKDRLKSALRKGHPQKAKTKCVFPVSVWL
RKETRV

| | | |
|---|---|---|
| NOV35b, CG54236-02 | SEQ ID NO: 443 | 1193 bp |
| DNA Sequence | ORF Start: ATG at 105 | ORF Stop: TAA at 1143 |

TGCTCCCTGTTTCATTAAAACCTAGAGAGATGTAATCAGTAAGCAAGAAGGAAAAAGGGAAATTCACA
AAGTAACTTTTTGTGTCTGTTTCTTTTTAACCCAGCATGGAGAGAAAATTTATGTCCTTGCAACCATC
CATCTCCGTATCAGAAATGGAACCAAATGGCACCTTCAGCAATAACAACAGCAGGAACTGCACAATTG
AAAACTTCAAGAGAGAATTTTTCCCAATTGTATATCTGATAATATTTTTCTGGGGAGTCTTGGGAAAT
GGGTTGTCCATATATGTTTTCCTGCAGCCTTATAAGAAGTCCACATCTGTGAACGTTTTCATGCTAAA
TCTGGCCATTTCAGATCTCCTGTTCATAAGCACGCTTCCCTTCAGGGCTGACTATTATCTTAGAGGCT
CCAATTGGATATTTGGAGACCTGGCCTGCAGGATTATGTCTTATTCCTTGTATGTCAACATGTACAGC
AGTATTTATTTCCTGACCGTGCTGAGTGTTGTGCGTTTCCTGGCAATGGTTCACCCCTTTCGGCTTCT
GCATGTCACCAGCATCAGGAGTGCCTGGATCCTCTGTGGGATCATATGGATCCTTATCATGGCTTCCT
CAATAATGCTCCTGGACAGTGGCTCTGAGCAGAACGGCAGTGTCACATCATGCTTAGAGCTGAATCTC
TATAAAATTGCTAAGCTGCAGACCATGAACTATATTGCCTTGGTGGTGGGCTGCCTGCTGCCATTTTT
CACACTCAGCATCTGTTATCTGCTGATCATTCGGGTTCTGTTAAAAGTGGAGGTCCCAGAATCGGGGC
TGCGGGTTTCTCACAGGAAGGCACTGACCACCATCATCATCACCTTGATCATCTTCTTCTTGTGTTTC
CTGCCCTATCACACACTGAGGACCGTCCACTTGACGACATGGAAAGTGGGTTTATGCAAAGACAGACT
GCATAAAGCTTTGGTTATCACACTGGCCTTGGCAGCAGCCAATGCCTGCTTCAATCCTCTGCTCTATT
ACTTTGCTGGGGAGAATTTTAAGGACAGACTAAAGTCTGCACTCAGAAAAGGCCATCCACAGAAGGCA
AAGACAAAGTGTGTTTTCCCTGTTAGTGTGTGGTTGAGAAAGGAAACAAGAGTATAAGGAGCTCTTAG
ATGAGACCTGTTCTTGTATCCTTGTGTCCATCTTCATTCACTCATAGTCTCCAAATGACTTTGTATTT
ACATCACTCCCAACAAATGTTGATTCTTAATATTTA

| | | | |
|---|---|---|---|
| NOV35b, CG54236-02 | SEQ ID NO: 444 | 346 aa | MW at 39634.7kD |
| Protein Sequence | | | |

MERKFMSLQPSISVSEMEPNGTFSNNNSRNCTIENFKREFFPIVYLIIFFWGVLGNGLSIYVFLQPYK
KSTSVNVFMLNLAISDLLFISTLPFRADYYLRGSNWIFGDLACRIMSYSLYVNMYSSIYFLTVLSVVR
FLAMVHPFRLLHVTSIRSAWILCGIIWILIMASSIMLLDSGSEQNGSVTSCLELNLYKIAKLQTMNYI
ALVVGCLLPFFTLSICYLLIIRVLLKVEVPESGLRVSHRKALTTIIITLIIFFLCFLPYHTLRTVHLT
TWKVGLCKDRLHKALVITLALAAANACFNPLLYYFAGENFKDRLKSALRKGHPQKAKTKCVFPVSVWL
RKETRV

A ClustalW comparison of the above protein sequences yields the following sequence alignment shown in Table 35B.

TABLE 35B
Comparison of the NOV35 protein sequences.

NOV35a MERKFMSLQPSISVSEMEPNGTFSNNNSRNCTIENFKREFFPIVYLIIFFWGVLGNGLSI
NOV35b MERKFMSLQPSISVSEMEPNGTFSNNNSRNCTIENFKREFFPIVYLIIFFWGVLGNGLSI
NOV35a YVFLQPYKKSTSVNVFMLNLAISDLLFISTLPFRADYYLRGSNWIFGDLACRIMSYSLYV
NOV35b YVFLQPYKKSTSVNVFMLNLAISDLLFISTLPFRADYYLRGSNWIFGDLACRIMSYSLYV
NOV35a NMYSSIYFLTVLSVVRFLAMVHPFRLLHVTSIRSAWILCGIIWILIMASSIMLLDSGSEQ
NOV35b NMYSSIYFLTVLSVVRFLAMVHPFRLLHVTSIRSAWILCGIIWILIMASSIMLLDSGSEQ
NOV35a NGSVTSCLELNLYKIAKLQTMNYIALVVGCLLPFFTLSICYLLIIRVLLKVEVPESGLRV
NOV35b NGSVTSCLELNLYKIAKLQTMNYIALVVGCLLPFFTLSICYLLIIRVLLKVEVPESGLRV
NOV35a SHRKALTTIIITLIIFFLCFLPYHTLRTVHLTTWKVGLCKDRLHKALVITLALAAANACF
NOV35b SHRKALTTIIITLIIFFLCFLPYHTLRTVHLTTWKVGLCKDRLHKALVITLALAAANACF
NOV35a NPLLYYFAGENFKDRLKSALRKGHPQKAKTKCVFPVSVWLRKETRV
NOV35b NPLLYYFAGENFKDRLKSALRKGHPQKAKTKCVFPVSVWLRKETRV
NOV35a (SEQ ID NO: 442)
NOV35b (SEQ ID NO: 444)

Further analysis of the NOV35a protein yielded the following properties shown in Table 35C.

TABLE 35C
Protein Sequence Properties NOV35a

| | |
|---|---|
| SignalP analysis: | Cleavage site between residues 60 and 61 |

PSORT II analysis:
PSG: a new signal peptide prediction method

N-region: length 4; pos.chg 2; neg.chg 1
H-region: length 11; peak value 5.18
PSG score: 0.78

GvH: von Heijne's method for signal seq. recognition

GvH score (threshold: −2.1): −0.37
possible cleavage site: between 55 and 56

>>> Seems to have a cleavable signal peptide (1 to 55)
ALOM: Klein et al's method for TM region allocation

| Init position for calculation: 56 | | |
|---|---|---|
| Tentative number of TMS(s) for the threshold 0.5: 6 | | |
| INTEGRAL | Likelihood = −2.81 | Transmembrane 75-91 |
| INTEGRAL | Likelihood = −3.72 | Transmembrane 125-141 |
| INTEGRAL | Likelihood = −8.86 | Transmembrane 157-173 |
| INTEGRAL | Likelihood = −7.75 | Transmembrane 204-220 |
| INTEGRAL | Likelihood = −11.36 | Transmembrane 245-261 |
| INTEGRAL | Likelihood = −1.06 | Transmembrane 287-303 |

| | |
|---|---|
| PERIPHERAL | Likelihood = 2.38 (at 222) |

ALOM score: −11.36 (number of TMSs: 6)

MTOP: Prediction of membrane topology (Hartmann et al.)

Center position for calculation: 27
Charge difference: 2.0 C(1.0) − N(−1.0)
C > N: C-terminal side will be inside

>>>Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75): | 11.72 |
| Hyd Moment(95): | 9.21 | G content: | 0 |
| D/E content: | 2 | S/T content: | 4 |

Score: −3.79

Gavel: prediction of cleavage sites for mitochondrial preseq

R-2 motif at 13 ERK|FM

NUCDISC: discrimination of nuclear localization signals

pat4: none
pat7: none
bipartite: none
content of basic residues: 10.4%
NLS Score: −0.47

KDEL: ER retention motif in the C-terminus: none
ER Membrane Retention Signals:

XXRR-like motif in the C-terminus: ERKF
KKXX-like motif in the C-terminus: KETR

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

66.7%: endoplasmic reticulum
11.1%: Golgi
11.1%: vacuolar
11.1%: cytoplasmic

>> prediction for CG54236-01 is end (k = 9)

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU11923 | Human G-protein coupled | 1 . . . 346 | 346/346 (100%) | 0.0 |
| | receptor HGPRBMY11v1 - | 1 . . . 346 | 346/346 (100%) | |
| | Homo sapiens, 346 aa. | | | |
| | [WO200286123-A2, 31-OCT- | | | |
| | 2002] | | | |
| ABP81707 | Human cysteinyl leukotriene | 1 . . . 346 | 346/346 (100%) | 0.0 |
| | CYSLT2 receptor protein SEQ | 1 . . . 346 | 346/346 (100%) | |
| | ID NO: 589 - Homo sapiens, 346 | | | |
| | aa. [WO200261087-A2, 08- | | | |
| | AUG-2002] | | | |
| ABB05229 | Human LTD4-like G protein- | 1 . . . 346 | 346/346 (100%) | 0.0 |
| | coupled receptor protein SEQ ID | 1 . . . 346 | 346/346 (100%) | |
| | NO: 2 - Homo sapiens, 346 aa. | | | |
| | [WO200194580-A1, 13-DEC- | | | |
| | 2001] | | | |
| AAG77965 | Human G-protein coupled | 1 . . . 346 | 346/346 (100%) | 0.0 |
| | receptor PFI-017* - Homo | 1 . . . 346 | 346/346 (100%) | |
| | sapiens, 346 aa. [US2001039037- | | | |
| | A1, 08-NOV-2001] | | | |
| AAE17231 | Human CysLT2 GPCR (G- | 1 . . . 346 | 346/346 (100%) | 0.0 |
| | protein coupled receptor) - Homo | 1 . . . 346 | 346/346 (100%) | |
| | sapiens, 346 aa. [WO200192302- | | | |
| | A2, 06-DEC-2001] | | | |

In a BLAST search of public sequence databases, the NOV35a protein was found to have homology to the proteins shown in the BLASTP data in Table 35E.

TABLE 35E
Public BLASTP Results for NOV35a

| | | NOV35a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NS75 | Cysteinyl leukotriene receptor 2 | 1 . . . 346 | 346/346 (100%) | 0.0 |
| | (CysLTR2) (PSEC0146) (HG57) | 1 . . . 346 | 346/346 (100%) | |
| | (HPN321) (hGPCR21) - Homo | | | |
| | sapiens (Human), 346 aa. | | | |
| CAC69290 | Sequence 1 from Patent | 1 . . . 346 | 344/346 (99%) | 0.0 |
| | WO0159118 - Homo sapiens | 1 . . . 346 | 345/346 (99%) | |
| | (Human), 346 aa. | | | |
| Q95N03 | Cysteinyl leukotriene receptor 2 | 1 . . . 346 | 275/347 (79%) | e−158 |
| | (CysLTR2) - Sus scrofa (Pig), | 1 . . . 345 | 300/347 (86%) | |
| | 345 aa. | | | |
| Q8R528 | Cysteinyl leukotriene 2 receptor - | 17 . . . 324 | 226/308 (73%) | e−132 |
| | Mus musculus (Mouse), 309 aa. | 1 . . . 308 | 256/308 (82%) | |
| Q920A1 | Cysteinyl leukotriene receptor 2 | 17 . . . 324 | 224/308 (72%) | e−131 |
| | (CysLTR2) - Mus musculus | 1 . . . 308 | 255/308 (82%) | |
| | (Mouse), 309 aa. | | | |

PFam analysis indicates that the NOV35a protein contains the domains shown in the Table 35F.

TABLE 35F
Domain Analysis of NOV35a

| | NOV35a | Identities/ | |
|---|---|---|---|
| | Match | Similarities | |
| Pfam Domain | Region | for the Matched Region | Expect Value |
| 7tm_1 | 55 . . . 305 | 89/277 (32%) | 6.1e−54 |
| | | 185/277 (67%) | |
| TAS2R | 33 . . . 324 | 64/314 (20%) | 0.019 |
| | | 173/314 (55%) | |

### Example 36

The NOV36 clone was analyzed, and the nucleotide and encoded polypeptide sequences are shown in Table 36A.

TABLE 36A
NOV36 Sequence Analysis

| | | |
|---|---|---|
| NOV36a, CG54479-06 | SEQ ID NO: 445 | 2066 bp |
| DNA Sequence | ORF Start: ATG at 22 | ORF Stop: TAG at 2032 |

ACAGGTTTCACAACTTCCCGGATGGGGCTGTGGTGGGTCAAGTGCAGCCTCCAGCCAGCCAGGATGGG
GTGGCTCCCACTCCTGCTGCTTCTGACTCAATGCTTAGGGGTCCCTGGGCAGCGCTCGCCATTGAATG
ACTTCCAAGTGCTCCGGGGCACAGAGCTACAGCACCTGCTACATGCGGTGGTGCCCGGGCCTTGGCAG
GAGGATGTGGCAGATGCTGAAGAGTGTGCTGGTCGCTGTGGGCCCTTAATGGACTGCCGGGCCTTCCA
CTACAACGTGAGCAGCCATGGTTGCCAACTGCTGCCATGGACTCAACACTCGCCCCACACGAGGCTGC
GGCGTTCTGGGCGCTGTGACCTCTTCCAGAAGAAAQACTACGTACGGACCTGCATCATGAACAATGGG
GTTGGGTACCGGGGCACCATGGCCACGACCGTGGGTGGCCTGCCCTGCCAGGCTTGGAGCCACAAGTT
CCCGAATGATCACAAGTACACGCCCACTCTCCGGAATGGCCTGGAAGAGAACTTCTGCCGTAACCCTG
ATGGCGACCCCGGAGGTCCTTGGTGCTACACAACAGACCCTGCTGTGCGCTTCCAGAGCTGCGGCATC
AAATCCTGCCGGGAGGCCGCGTGTGTCTGGTGCAATGGCGAGGAATACCGCGGCGCGGTAGACCGCAC
GGAGTCAGGGCGCGAGTGCCAGCGCTGGGATCTTCAGCACCCGCACCAGCACCCCTTCGAGCCGGGCA
AGTTCCTCGACCAAGGTCTGGACGACAACTATTGCCGGAATCCTGACGGCTCCGAGCGGCCATGGTGC
TACACTACGGATCCGCAGATCGAGCGAGAGTTCTGTGACCTCCCCCGCTGCGGGTCCGAGGCACAGCC
CCGCCAAGAGGCCACAACTGTCAGCTGCTTCCGCGGGAAGGGTGAGGGCTACCGGGGCACAGCCAATA
CCACCACTGCGGGCGTACCTTGCCAGCGTTGGGACGCGCAAATCCCTCATCAGCACCGATTTACGCCA
GAAAAATACGCGTGCAAAGACCTTCGGGAGAACTTCTGCCGGAACCCCGACGGCTCAGAGGCGCCCTG
GTGCTTCACACTGCGGCCCGGCATGCGCGCGGCCTTTTGCTACCAGATCCGGCGTTGTACAGACGACG
TGCGGCCCCAGGACTGCTACCACGGCGCAGGGGAGCAGTACCGCGGCACGGTCAGCAAGACCCGCAAG
GGTGTCCAGTGCCAGCGCTGGTCCGCTGAGACGCCGCACAAGCCGCAGTTCACGTTTACCTCCGAACC
GCATGCACAACTGGAGGAGAACTTCTGCCGGAACCCAGATGGGGATAGCCATGGGCCCTGGTGCTACA
CGATGGACCCAAGGACCCCATTCGACTACTGTGCCCTGCGACGCTGCGCTGATGACCAGCCGCCATCA
ATCCTGGACCCCCCAGACCAGGTGCAGTTTGAGAAGTGTGGCAAGAGGGTGGATCGGCTGGATCAGCG
GCGTTCCAAGCTGCGCGTGGTTGGGGGCCATCCGGGCAACTCACCCTGGACAGTCAGCTTGCGGAATC
GGCAGGGCCAGCATTTCTGCGGGGGGTCTCTAGTGAAGGAGCAGTGGATACTGACTGCCCGGCAGTGC
TTCTCCTCCTGCCATATGCCTCTCACGGGCTATGAGGTATGGTTGGGCACCCTGTTCCAGAACCCACA
GCATGGAGAGCCAAGCCTACAGCGGGTCCCAGTAGCCAAGATGGTGTGTGGGCCCTCAGGCTCCCAGC
TTGTCCTGCTCAAGCTGGAGAGATCTGTGACCCTGAACCAGCGTGTGGCCCTGATCTGCCTGCCCCCT
GAATGGTATGTGGTGCCTCCAGGGACCAAGTGTGAGGGTGACTACGGGGGCCCACTTGCCTGCTTTAC
CCACAACTGCTGGGTCCTGGAAGGAATTATAATCCCCAACCGAGTATGCGCAAGGTCCCGCTGGCCAG
CTGTCTTCACGCGTGTCTCTGTGTTTGTGGACTGGATTCACAAGGTCATGAGACTGGGTTAGGCCCAG
CCTTGATGCCATATGCCTTGGGGAGG

| | | | |
|---|---|---|---|
| NOV36a, CG54479-06 | SEQ ID NO: 446 | 670 aa | MW at 76160.6kD |
| Protein Sequence | | | |

MGLWWVTVQPPARRNGWLPLLLLLTQCLGVPGQRSPLWDFQVLRGTELQHLLHAVVPGPWQEDVADAE
ECAGRCGPLMDCRAFHYNVSSHGCQLLPWTQHSPHTRLRRSGRCDLFQKKDYVRTCIMNNGVGYRGTM
ATTVGGLPCQAWSHKFPMDHKYTPTLRNGLEENFCRNPDGDPGGPWCYTTDPAVRFQSCGIKSCREAA
CVWCNGEEYRGAVDRTESGRECQRWDLQHPHQHPFEPGKFLDQGLDDNYCRNPDGSERPWCYTTDPQI
EREFCDLPRCGSEAQPRQEATTVSCFRGKGEGYRGTANTTTAGVPCQRWDAQIPHQHRFTPEKYACKD
LRENFCRNPDGSEAPWCFTLRPGMRAAFCYQIRRCTDDVRPQDCYHGAGEQYRGTVSKTRKGVQCQRW
SAETPHKPQFTFTSEPHAQLEENFCRNPDGDSHGPWCYTMDPRTPFDYCALRRCADDQPPSILDPPDQ
VQFEKCGKRVDRLDQRRSKLRVVGGHPGNSPWTVSLRNRQGQHFCGGSLVKEQWILTARQCFSSCHMP
LTGYEVWLGTLFQNPQHGEPSLQRVPVAKMVCGPSGSQLVLLKLERSVTLNQRVALICLPPEWYVVPP
GTKCEGDYGGPLACFTHMCWVLEGIIIPNRVCARSRWPAVFTRVSVFVDWIHKVMRLG

| | | |
|---|---|---|
| NOV36b, CG54479-05 | SEQ ID NO: 447 | 1698 bp |
| DNA Sequence | ORF Start: ATG at 1 | ORF Stop: TGA at 1696 |

ATGACTTCTAGGTGCTCCGGGGCACAGAGCTACCTACAAGCGGTGGTGCCCGGGCCTTGGCAGGAGGA
TGTGGCAGATGCTGAAGAGTGTGCTGGTCGCTGTGGGCCCTTAATGGACTGCGCGTTCCACTACAATG
TGAGCAGCCATGGTTGCCAACTGCTGCCATGGACTCAACACTCACCCCACACGAGGCTGCGGCATTCT
GGGCGCTGTGACCTCTTCCAGGAGAAAGACTACATACGGACCTGCATCATGAACAATGGGGTTGGGTA
CCGGGGCACCATGGCCACGACCGTGGGTGGCCTGTCCTGCCAGGCTTGGAGCCACAAGTTCCCGAACG
ATCACCAGTACATGCCCACGCTCCGGAATGGCCTGGAAGAGAACTTCTGCCGTAACCCTGATGGCGAC
CCCGGAGGTCCTTGGTGCCACACAACAGACCCTGCCGTGCGCTTCCAGAGCTGCGGCATCAAATCCTG
CCGGGTGGCCGCGTGTGTCTGGTGCAATGGCGAGGAATACCGCGGCGCGGTAGACCGCACCGAGTCAG
GGCGCGAGTGCCAGCGCTGGGATCTTCAGCACCCGCACCAGCACCCCTTCGAGCCGGGCAAGTTCCTC
GACCAAGGTCTGGACGACAACTATTGCCGGAATCCTGACGGCTCCGAGCGGCCATGGTGCTACACTAC
GGATCCGCAGATCGAGCGAGAATTCTGTGACCTCCCCCGCTGCGGTTCCGAGGCACAGCCCCGCCAAG
AGGCCACAAGTGTCAGCTGCTTCCGCGGGAAGGGTGAGGGCTACCGGGGCACAGCCAATACCACCACC
GCGGGCGTACCTTGCCAGCGTTGGGACGCGCAAATCCCGCATCAGCACCGATTTACGCCAGAAAAATA
CGCGTGCAAGGACCTTCGGGAGAACTTCTGCCGGAACCCCGACGGCTCAGAGGCGCCCTGGTGCTTCA
CACTGCGGCCCGGCATGCGCGTGGGCTTTTGCTACCAGATCCGGCGTTGTACAGACGACGTGCGGCCC
CAGGACTGCTACCACGGCGCGGGGGAGCAGTACCGCGGCACGGTCAGCAAGACCCGCAAGGGTGTCCA
GTGCCAGCGCGGGTCCGCTGAGACGCCGCACAAGCCGCAGTTCACGTTTACCTCCGAACCGCATGCAC
AACTGGAGGAGAACTTCTGCCAGGACCCAGATGGGGATAGCCATGGGCCCTGGTGCTACACGATGGAC
CCAAGGACCCCATTCGACTACTGTGCCCTGCGACGCTGCGCTGATGACCAGCCGCCATCAATCCTGGA
CCCCCCCGACCAGGTGCAGTTTGAGAAGTGTGGCAAGAGGGTGGATCGGCTGGATCAGCGTTGTTCCA
AGCTGCGCGTGGCTGGGGGCCATCCGGGCAACTCACCCTGGACAGTCAGCTTGCGGAATAGGCAGGGC
CAGCATTTCTGCGGGGGGTCTCTAGTGAAGGAGCAGTGGATACTGACTGCCCGGCAGTGCTTCTCCTC
CAGCCATATGCCTCTCACGGGCTATGAGGTATGGTTGGGCACCCTGTTCCAGAACCCACAACATGGAG
AGCCAGGCCTACAGCGGGTCCCAGTAGCCAAGATGCTGTGTGGGCCCTCAGGCTCTCAGCTTGTCCTG
CTCAAGCTGGAGAGGTCTGTGACCCTGAACCAGCGTGTGGCCCTGATCTGCCTGCCGCCTGAATGA

| | | | |
|---|---|---|---|
| NOV36b, CG54479-05 | SEQ ID NO: 448 | 565 aa | MW at 63751.8kD |
| Protein Sequence | | | |

MTSRCSGAQSYLQAVVPGPWQEDVADAEECAGRCGPLMDCAFHYNVSSHGCQLLPWTQHSPHTRLRHS
GRCDLFQEKDYIRTCIMNNGVGYRGTMATTVGGLSCQAWSHKFPNDHQYMPTLRNGLEENFCRNPDGD
PGGPWCHTTDPAVRFQSCGIKSCRVAACVWCNGEEYRGAVDRTESGRECQRWDLQHPHQHPFEPGKFL
DQGLDDNYCRNPDGSERPWCYTTDPQIEREFCDLPRCGSEAQPRQEATSVSCFRGKGEGYRGTANTTT
AGVPCQRWDAQIPHQHRFTPEKYACKDLRENFCRNPDGSEAPWCFTLRPGMRVGFCYQIRRCTDDVRP
QDCYHGAGEQYRGTVSKTRKGVQCQRGSABTPHKPQFTFTSEPHAQLEENFCQDPDGDSHGPWCYTND
PRTPFDYCALRRCADDQPPSILDPPDQVQFEKCGKRVDRLDQRCSKLRVAGGHPGNSPWTVSLRNRQG
QHFCGGSLVKEQWILTARQCFSSSHMPLTGYEVWLGTLFQNPQHGEPGLQRVPVAKMLCGPSGSQLVL
LKLERSVTLNQRVALICLPPE

| | | |
|---|---|---|
| NOV36c, CG54479-01 | SEQ ID NO: 449 | 2200 bp |
| DNA Sequence | ORF Start: ATG at 21 | ORF Stop: TAG at 2157 |

TGCAGCCTCCAGCCAGAAGGATGGGGTGGCTCCCACTCCTGCTGCTTCTGACTCAATGCTTAGGGGTC
CCTGGGCAGCGCTCGCCATTGAATGACTTCGAGGTGCTCCGGGGCACAGAGCTACAGCGGCTGCTACA
AGCGGTGGTGCCCGGGCCTTGGCAGGAGGATGTGGCAGATGCTGAAGAGTGTGCTGGTCGCTGTGGGC
CCTTAATGGACTGCCGGGCGTTCCACTACAATGTGAGCAGCCATGGTTGCCAACTGCTGCCATGGACT
CAACACTCACCCCACACGAGGCTGCGGCATTCTGGGCGCTGTGACCTCTTCCAGGAGAAAGACTACAT
ACGGACCTGCATCATGAACAATGGGGTTGGGTACCGGGGCACCATGGCCACGACCGTGGGTGGCCTGT
CCTGCCAGGCTTGGAGCCACAAGTTCCCGAACGATCACAGGTACATGCCCACGCTCCGGAATGGCCTG
GAAGAGAACTTCTGCCGTAACCCTGATGGCGACCCCGGAGGTCCTTGGTGCCACACAACAGACCCTGC
CGTGCGCTTCCAGAGCTGCGGCATCAAATCCTGCCGGTCTGCCGCGTGTGTCTGGTGCAATGGCGAGG
AATACCGCGGCGCGGTAGACCGCACCGAGTCAGGGCGCGAGTGCCAGCGCTGGGATCTTCAGCACCCG
CACCAGCACCCCTTCGAGCCGGGCAAGTACCCCGACCAAGGTCTGGACGACAACTATTGCCGGAATCC
TGACGGCTCCGAGCGGCCATGGTGCTACACTACGGATCCGCAGATCGAGCGAGAATTCTGTGACCTCC
CCCGCTGCGGTTCCGAGGCACAGCCCCGCCAAGAGGCCACAAGTGTCAGCTGCTTCCGCGGGAAGGGT
GAGGGCTACCGGGGCACAGCCAATACCACCACCGCGGGCGTACCTTGCCAGCGTTGGGACGCGCAAAT
CCCGCATCAGCACCGATTTACGCCAGAAAAATACGCGTGCAAGGACCTTCGGGAGAACTTCTGCCGGA
ACCCCGACGGCTCAGAGGCGCCCTGGTGCTTCACACTGCGGCCCGGCATGCGCGTGGGCTTTTGCTAC
CAGATCCGGCGTTGTACAGACGACGTGCGGCCCCAGGGTTGCTACCACGGCGCGGGGGAGCAGTACCG
CGGCACGGTCAGCAAGACCCGCAAGGGTGTCCAGTGCCAGCGCGCGTCCGCTGAGACGCCGCACAAGC
CGCAGTTTACCTTTACCTCCGAACCGCATGCACAACTGGAGGAGAACTTCTGCCGCGACCCAGATGGG
GATAGCTATGGGCCCTGGTGCTACACGATGGACCCAAGGACCCCATTCGACTACTGTGCCCTGCGACG
CTGCGCTGATGACCAGCCGCCATCAATCCTGGACCCCCCCGACCAGGTGCAGTTTGAGAAGTGTGGCA
AGAGGGTGGATCGGCTGGATCAGCGTTGTTCCAAGCTGCGCGTGGCTGGGGGCCATCCGGGCAACTCA
CCCTGGACAGTCAGCTTGCGGAATAGGCAGGGCCAGCATTTCTGCGGGGGGTCTCTAGTGAAGGAGCA
GTGGATACTGACTGCCCGGCAGTGCTTCTCCTCCAGCCATATGCCTCTCACGGGCTATGAGGTATGGT
TGGGCACCCTGTTCCAGAACCCACAACATGGAGAGCCAGGCCTACAGCGGGTCCCAGTAGCCAAGATG
CTGTGTGGGCCCTCAGGCTCTCAGCTTGTCCTGCTCAAGCTGGAGAGATCTGTGACCCTGAACCAGCG
TGTGGCCCTGATCTGCCTGCCGCCTGAATGGTATGTGGTGCCTCCAGGGACCAAGTGTGAGATTGCAG
GCCGGGGTGAGACCAAAGGTACGGGTAATGACACAGTCCTAAATGTGGCCTTGCTGAATGTCATCTCC
AACCAGGAGTGTAACATCAAGCACCGAGGACATGTGCGGGAGAGCGAGATGTGCACTGAGGGACTGTT
GGCCCCTGTGGGGGCCTGTGAGGGGGGTGACTACGGGGGCCCACTTGCCTGCTTTACCCACAACTGCT
GGGTCCTGGAAGGAATTAGAATCCCCAACCGAGTATGCGCAAGGTCGCGCTGGCCAGCCGTCTTCACA
CGTGTCTCTGTGTTTGTGGACTGGATTCACAAGGTCATGAGACTGGGTTAGGCCCAGCCTTGACGCCA
TATGCTTTGGGGAGGACAAAACTT

| | | | |
|---|---|---|---|
| NOV36c, CG54479-01 | SEQ ID NO: 450 | 712 aa | MW at 80097.8kD |
| Protein Sequence | | | |

MGWLPLLLLLTQCLGVPGQRSPLNDFEVLRGTELQRLLQAVVPGPWQEDVADAEECAGRCGPLMDCRA
FHYNVSSHGCQLLPWTQHSPHTRLRHSGRCDLFQEKDYIRTCIMNNGVGYRGTMATTVGGLSCQAWSH
KFPNDHRYMPTLRNGLEENFCRNPDGDPGGPWCHTTDPAVRFQSCGIKSCRSAACVWCNGEEYRGAVD
RTESGRECQRWDLQHPHQHPFEPGKYPDQGLDDNYCRNPDGSERPWCYTTDPQIEREFCDLPRCGSEA
QPRQEATSVSCFRGKGEGYRGTANTTTAGVPCQRWDAQIPHQHRFTPEKYACKDLRENFCRNPDGSEA
PWCFTLRPGMRVGFCYQIRRCTDDVRPQGCYHGAGEQYRGTVSKTRKGVQCQRASAETPHKPQFTFTS
EPHAQLEENFCRDPDGDSYGPWCYTMDPRTPFDYCALRRCADDQPPSILDPPDQVQFEKCGKRVDRLD
QRCSKLRVAGGHPGNSPWTVSLRNRQGQHFCGGSLVKEQWILTARQCFSSSHMPLTGYEVWLGTLFQN
PQHGEPGLQRVPVAKMLCGPSGSQLVLLKLERSVTLNQRVALICLPPEWYVVPPGTKCEIAGRGETKG
TGNDTVLNVALLNVISNQECNIKHRGHVRESEMCTEGLLAPVGACEGGDYGGPLACFTHNCWVLEGIR
IPNRVCARSRWPAVFTRVSVFVDWIHKVMRLG

| | | |
|---|---|---|
| NOV36d, CG54479-02 | SEQ ID NO: 451 | 1710 bp |
| DNA Sequence | ORF Start: ATG at 1 | ORF Stop: TGA at 1705 |

ATGACTTCCAGGTGCTCCGGGGCACAGAGCTACCTGCTACATGCGGTGGTGCCTGGGCCTTGGCAGGA
GGATGTGGCAGATGCTGAAGAGTGTGCTGGTCGCTGTGGGCCCTTAACGGACTGCTGGGCCTTCCACT
ACAATGTGAGCAGCCATGGTTGCCAACTGCTGCCATGGACTCAACACTCGCCCCACTCAAGGCTGTGG
CATTCTGGGCGCTGTGACCTCTTCCAGAAGAAAGACTACATACGGACCTGCATCATGAACAATGGGGT
TGGGTACCGGGGCACCATGGCCACGACCGTGGGTGGCCTGTCCTGCCAGGCTTGGAGCCACAAGTTCC
CGAATGATCACAAGTACATGCCCACGCTCCGGAATGGCCTGGAAGAGAACTTCTGCCATAACCCTGAT
GGCGACCCCGGAGGTCCTTGGTGCCACACAACAGACCCTGCCGTGCGCTTCCAGAGCTGCGGCATCAA
ATCCTGCCGGGTGGCCGCGTGTGTCTGGTGCAATGGCGAGGAATACCGCGGCGCGGTAGACCGCACCG
AGTCAGGGCGCGAGTGCCAGCGCTGGGATCTTCAGCACCCGCACCAGCACCCCTTCGAGCCGGGCAAG
TACCTCGACCAAGGTCTGGACGACAACTATTGCCGGAATCCTGACGGCTCCGAGCGGCCATGGTGCTA
CACTACGGATCCGCAGATCGAGCGAGAATTCTGTGACCTCCCCCGCTGCGGTTCCGAGGCACAGCCCC
GCCAAGAGGCCACAAGTGTCAGCTGCTTCCGCGGGAAGGGTGAGGGCTACCGGGGCACAGCCAATACC
ACCACCGCGGGCGTACCTTGCCAGCGTTGGGACGCGCAAATCCCGCATCAGCACCGATTTACGCCAGA
AAAATACGCGTGCAAGGACCTTCGGGAGAACTTCTGCCGGAACCCCGACGGCTCAGAGGCGCCCTGGT
GCTTCACACTGCGGCCCGGCATGCGCGTGGGCTTTTGCTACCAGATCCGGCGTTGTACAGACGACGTG
CGGCCCCAGGACTGCTACCACGGCGCGGGGGAGCAGTACCGCGGCACGGTCAGCAAGACCCGCAAGGG
TGTCCAGTGCCAGCGCGCGTCCGCTGAGACGCCGCACAAGCCGCAGTTCACGTTTACCTCCGAACCGC
ATGCACAACTGGAGGAGAACTTCTGCCAGGACCCAGATGGGGATAGCCATGGGCCCTGGTGCTACACG
ATGGACCCAAGGACCCCATTCGACTACTGTGCCCTGCGACGCTGCGCTGATGACCAGCCGCCATCAAT
CCTGGACCCCCCCACAGACCAGGTGCAGTTTGAGAAGTGTGGCAAGAGGGTGGATCGGCTGGATCAGC
GTCGTTCCAAGCTGCGCGTGGCTGGGGGCCATCCGGGCAACTCACCCTGGACAGTCAGCTTGGGGAAT
CGGAGGCAGGGCCAGCATTTCTGCGGGGGGTCTCTAGTGAAGGAGCAGTGGATACTGACTGCCCGGCA
GTGCTTCTCCTCCCATATGCCTCTCACGGGCTATGAGGTATGGTTGGGCACCCTGTTCCAGAACCCAC
AACATGGAGAGCCAGGCCTACAGCGGGTCCCAGTAGCCAAGATGCTGTGTGGGCCCTCAGGCTCCCAG
CTTGTCCTGCTCAAGCTGGAGAGATCTGTGACCCTGAACCAGCGTGTGGCCCTGATCTGCCTGCCGCC
TGAATGATAT

| | | | |
|---|---|---|---|
| NOV36d, CG54479-02 | SEQ ID NO: 452 | 568 aa | MW at 64180.3kD |
| Protein Sequence | | | |

MTSRCSGAQSYLLHAVVPGPWQEDVADAEECAGRCGPLTDCWAFHYNVSSHGCQLLPWTQHSPHSRLW
HSGRCDLFQKKDYIRTCIMNNGVGYRGTMATTVGGLSCQAWSHKFPNDHKYMPTLRNGLEENFCHNPD
GDPGGPWCHTTDPAVRFQSCGIKSCRVAACVWCNGEEYRGAVDRTESGRECQRWDLQHPHQHPFEPGK
YLDQGLDDNYCRNPDGSERPWCYTTDPQIEREFCDLPRCGSEAQPRQEATSVSCFRGKGEGYRGTANT
TTAGVPCQRWDAQIPHQHRFTPEKYACKDLRENFCRNPDGSEAPWCFTLRPGMRVGFCYQIRRCTDDV
RPQDCYHGAGEQYRGTVSKTRKGVQCQRASAETPHKPQFTFTSEPHAQLEENFCQDPDGDSHGPWCYT
MDPRTPFDYCALRRCADDQPPSILDPPTDQVQFEKCGKRVDRLDQRRSKLRVAGGHPGWSPWTVSLGN
RRQGQHFCGGSLVKEQWILTARQCFSSHMPLTGYEVWLGTLFQNPOHGEPGLQRVPVAKMLCGPSGSQ
LVLLKLERSVTLNQRVALICLPPE

| | | |
|---|---|---|
| NOV36e, CG54479-03 | SEQ ID NO: 453 | 1011 bp |
| DNA Sequence | ORF Start: at 7 | ORF Stop: at 1006 |

AAGCTTTGCATCATGAACAATGGGGTTGGGTACCGGGGCACCATGGCCACGACCGTGGGTGGCCTGCC
CTGCCAGGCTTGGAGCCACAAGTTCCCAAATGATCACAAGTACACGCCCACTCTCCGGAATGGCCTGG
AAGAGAACTTCTGCCGTAACCCTGATGGCGACCCCGGAGGTCCTTGGTGCTACACAACAGACCCTGCT
GTGCGCTTCCAGAGCTGCGGCATCGAATCCTGCCGGGAGGCCGCGTGTGTCTGGTGCAATGGCGAGGA
ATACCGCGGCGCGGTAGACCGCACGGAGTCAGGGCGCGAGTGCCAGCGCTGGGATCTTCAGCACCCGC
ACCAGCACCCCTTCGAGCCGGGCAAGTTCCTCGACCAAGGTCTGGACGACAACTATTGCCGGAATCCT
GACGGCTCCGAGCGGCCATGGTGCTACACTACGGATCCGCAGATCGAGCGAGAGTTCTGTGACCTCCC
CCGCTGCGGGTCCGAGGCACAGCCCCGCCAAGAGGCCACAACTGTCAGCTGCTTCCGCGGGAAGGGTG
AGGGCTACCGGGGCACAGCCAATACCACCACTGCGGGCGTACCTTGCCAGCGTTGGGACGCGCAAATC
CCTCATCAGCACCGATTTACGCCAGAAAAATACGCGTGCAAAGACCTTCGGGAGAACTTCTGCCGGAA
CCCCGACGGCTCAGAGGCGCCCTGGTGCTTCACACTGCGGCCCGGCATGCGCGCGGCCTTTTGCTACC
AGATCCGGCGTTGTACAGACGACGTGCGGCCCCAGGGGGAGCAGTACCGCGGCACGGTCAGCAAGACC
CGCAAGGGTGTCCAGTGCCAGCGCTGGTCCGCTGAGACGCCGCACAAGCCGCAGTTCACGTTTACCTC
CGAACCGCATGCACAACTGGAGGAGAACTTCTGCCGGAACCCAGATGGGGATAGCCATGGGCCCTGGT
GCTACACGATGGACCCAAGGACCCCATTCGACTACTGTGCCCTGCGACGCTGCCTCGAG

| | | | |
|---|---|---|---|
| NOV36e, CG54479-03 | SEQ ID NO: 454 | 333 aa | MW at 38129.9kD |
| Protein Sequence | | | |

CIMNNGVGYRGTMATTVGGLPCQAWSHKFPNDHKYTPTLRNGLEENFCRNPDGDPGGPWCYTTDPAVR
FQSCGIESCREAACVWCNGEEYRGAVDRTESGRECQRWDLQHPHQHPFEPGKFLDQGLDDNYCRNPDG
SERPWCYTTDPQIEREFCDLPRCGSEAQPRQEATTVSCFRGKGEGYRGTANTTTAGVPCQRWDAQIPH
QHRFTPEKYACKDLRENFCRNPDGSEAPWCFTLRPGMRAAFCYQIRRCTDDVRPQGEQYRGTVSKTRK
GVQCQRWSAETPHKPQFTFTSEPHAQLEENFCRNPDGDSHGPWCYTMDPRTPFDYCALRRC

| | | |
|---|---|---|
| NOV36f, GG54479-04 | SEQ ID NO: 455 | 1881 bp |
| DNA Sequence | ORF Start: ATG at 76 | ORF Stop: TGA at 1777 |

ACACATTACTGACATGTATGCCCACCTGACCTGCACCCACTCATGCCCACTCTGCAGGGCAGCGCTCG
+E,unc CCATTGAATGACTTCCAGGTGCTCCGGGGCACAGAGCTACCTGCTACATGCGGTGGTGCCTGGGCCTT
GGCAGGAGGATGTGGCAGATGCTGAAGAGTGTGCTGGTCGCTGTGGGCCCTTAACGGACTGCTGGGCC
TTCCACTACAATGTGAGCAGCCATGGTTGCCAACTGCTGCCATGGACTCAACACTCGCCCCACTCAAG
GCTGTGGCATTCTGGGCGCTGTGACCTCTTCCAGAAGAAAGACTACATACGGACCTGCATCATGAACA
ATGGGGTTGGGTACCGGGGCACCATGGCCACGACCGTGGGTGGCCTGTCCTGCCAGGCTTGGAGCCAC
AAGTTCCCGAATGATCACAAGTACATGCCCACGCTCCGGAATGGCCTGGAAGAGAACTTCTGCCATAA
CCCTGATGGCGACCCCGGAGGTCCTTGGTGCCACACAACAGACCCTGCCGTGCGCTTCCAGAGCTGCG
GCATCAAATCCTGCCGGGTGGCCGCGTGTGTCTGGTGCAATGGCGAGGAATACCGCGGCGCGGTAGAC
CGCACCGAGTCAGGGCGCGAGTGCCAGCGCTGGGATCTTCAGCACCCGCACCAGCACCCCTTCGAGCC
GGGCAGGTTCCTCGACCAAGGTCTGGACGACAACTATTGCCGGAATCCTGACGGCTCCGAGCGGCCAT
GGTGCTACACTACGGATCCGCAGATCGAGCGAGAATTCTGTGACCTCCCCCGCTGCGGTTCCGAGGCA
CAGCCCCGCCAAGAGGCCACAAGTGTCAGCTGCTTCCGCGGGAAGGGTGAGGGCTACCGGGGCACAGC
CAATACCACCACCGCGGGCGTACCTTGCCAGCGTTGGGACGCGCAAATCCCGCATCAGCACCGATTTA
CGCCAGAAAAATACGCGTGCAAGGACCTTCGGGAGAACTTCTGCCGGAACCTCGACGGCTCAGAGGCG
CCCTGGTGCTTCACACTGCGGCCCGGCATGCGCGTGGGCTTTTGCTACCAGATCCGGCGTTGTACAGA
CGACGTGCGGCCCCAGGACTGCTACCACGGCGCGGGGGAGCAGTACCGCGGCACGGTCAGCAAGACCC
GCAAGGGTGTCCAGTGCCAGCGCGCGTCCGCTGAGACGCCGCACAAGCCGCAGTTCACGTTTACCTCC
GAACCGCATGCACAACTGGAGGAGAACTTCTGCCAGACCCCAGATGGGGATAGCCATGGGCCCTGGTG
CTACACGATGGACCCAAGGACCCCATTCGACTACTGTGCCCTGCGACGCTGCGCTGATGACCAGCCGC
CATCAATCCTGGACCCCCCCGACCAGGTGCAGTTTGAGAAGTGTGGCAAGAGGGTGGATCGGCTGGAT
CAGCGTCGTTCCAAGCTGCGCGTGGCTGGGGGCCATCCGGGCAACTCACCCTGGACAGTCAGCTTGGG
GAATCGGCAGGGCCAGCATTTCTGCGGGGGGTCTCTAGTGAAGGAGCAGTGGATACTGACTGCCCGGC
AGTGCTTCTCCTCCCAGCATATGCCTCTCACGGGCTATGAGGTATGGTTGGGCACCCTGTTCCAGAAC
CCACAACATGGAGAGCCAGGCCTACAGCGGGTCCCAGTAGCCAAGATGCTGTGTGGGCCCTCAGGCTC
CCAGCTTGTCCTGCTCAAGCTGGAGAGGTCTGTGACCCTGAACCAGCGTGTGGCCCTGATCTGCCTGC
CGCCTGAATGATATGTGGTGCCTCCAGGGACCAAGTGTGAGATTGCAGGCCGGGGTGAGACCAAAGGT
AAGAGCATAGTGCACAGGACTGCTGGTGGCCAGGAGGCCCAGCCC

| | | | |
|---|---|---|---|
| NOV36f, CG54479-04 | SEQ ID NO: 456 | 567 aa | MW at 64065.2kD |
| Protein Sequence | | | |

MTSRCSGAQSYLLHAVVPGPWQEDVADAEECAGRCGPLTDCWAFHYNVSSHGCQLLPWTQHSPHSRLW
HSGRCDLFQKKDYIRTCIMNNGVGYRGTMATTVGGLSCQAWSHKFPNDHKYMPTLRNGLEENFCHNPD
GDPGGPWCHTTDPAVRFQSCGIKSCRVAACVWCNGEEYRGAVDRTESGRECQRWDLQHPHQHPFEPGR
FLDQGLDDNYCRNPDGSERPWCYTTDPQIEREFCDLPRCGSEAQPRQEATSVSCFRGKGEGYRGTANT
TTAGVPCQRWDAQIPHQHRFTPEKYACKDLRENFCRNLDGSEAPWCFTLRPGMRVGFCYQIRRCTDDV
RPQDCYHGAGEQYRGTVSKTRKGVQCQRASAETPHKPQFTFTSEPHAQLEENFCQTPDGDSHGPWCYT
MDPRTPFDYCALRRCADDQPPSILDPPDQVQFEKCGKRVDRLDQRRSKLRVAGGHPGNSPWTVSLGNR
QGQHFCGGSLVKEQWILTARQCFSSQHMPLTGYEVWLGTLFQNPQHGEPGLQRVPVAKMLCGPSGSQL
VLLKLERSVTLNQRVALICLPPE

A ClustalW comparison of the above protein sequences yields the following sequence alignment shown in Table 36B.

TABLE 36B
Comparison of the NOV36 protein sequences.

NOV36a MGLWWVTVQPPARRMGWLPLLLLLTQCLGVPGQRSPLNDFQVLRGTELQHLLHAVVPGPW
NOV36b ----------------------MTSRCSGAQSY------------------LQAVVPGPW
NOV36c --------------MGWLPLLLLLTQCLGVPGQRSPLWDFEVLRGTELQRLLQAVVPGPW
NOV36d ----------------------MTSRCSGAQSYL-----------------LHAVVPGPW
NOV36e ------------------------------------------------------------
NOV36f ----------------------MTSRCSGAQSYL-----------------LHAVVPGPW
NOV36a QEDVADAEECAGRCGPLMDCRAFHYNVSSHGCQLLPWTQHSPHTRLRRSGRCDLFQKKDY
NOV36b QEDVADAEECAGRCGPLMDC-AFHYNVSSHGCQLLPWTQHSPHTRLRHSGRCDLFQEKDY
NOV36c QEDVADAEECAGRCGPLMDCRAFHYNVSSHGCQLLPWTQHSPHTRLRHSGRCDLFQEKDY
NOV36d QEDVADAEECAGRCGPLTDCWAFHYNVSSHGCQLLPWTQHSPHSRLWHSGRCDLFQKKDY
NOV36e ------------------------------------------------------------
NOV36f QEDVADAEECAGRCGPLTDCWAFHYNVSSHGCQLLPWTQHSPHSRLWHSGRCDLFQKKDY
NOV36a VRTCIMNNGVGYRGTMATTVGGLPCQAWSHKFPNDHKYTPTLRNGLEENFCRNPDGDPGG
NOV36b IRTCIMNNGVGYRGTMATTVGGLSCQAWSHKFPNDHQYMPTLRNGLEENFCRNPDGDPGG
NOV36c IRTCIMNNGVGYRGTMATTVGGLSCQAWSHKFPNDHRYMPTLRNGLEENFCRNPDGDPGG
NOV36d IRTCIMNNGVGYRGTMATTVGGLSCQAWSHKFPNDHKYMPTLRNGLEENFCHNPDGDPGG
NOV36e ---CIMNNGVGYRGTMATTVGGLPCQAWSHKFPNDHKYTPTLRNGLEENFCRNPDGDPGG
NOV36f IRTCIMNNGVGYRGTMATTVGGLSCQAWSHKFPNDHKYMPTLRNGLEENFCHNPDGDPGG
NOV36a PWCYTTDPAVRFQSCGIKSCREAACVWCNGEEYRGAVDRTESGRECQRWDLQHPHQHPFE
NOV36b PWCHTTDPAVRFQSCGIKSCRVAACVWCNGEEYRGAVDRTESGRECQRWDLQHPHQHPFE
NOV36c PWCHTTDPAVRFQSCGIKSCRSAACVWCNGEEYRGAVDRTESGRECQRWDLQHPHQHPFE
NOV36d PWCHTTDPAVRFQSCGIKSCRVAACVWCNGEEYRGAVDRTESGRECQRWDLQHPHQHPFE
NOV36e PWCYTTDPAVRFQSCGIESCREAACVWCNGEEYRGAVDRTESGRECQRWDLQHPHQHPFE
NOV36f PWCHTTDPAVRFQSCGIKSCRVAACVWCNGEEYRGAVDRTESGRECQRWDLQHPHQHPFE
NOV36a PGKFLDQGLDDNYCRNPDGSERPWCYTTDPQIEREFCDLPRCGSEAQPRQEATTVSCFRG
NOV36b PGKFLDQGLDDNYCRNPDGSERPWCYTTDPQIEREFCDLPRCGSEAQPRQEATSVSCFRG
NOV36c PGKYPDQGLDDNYCRNPDGSERPWCYTTDPQIEREFCDLPRCGSEAQPRQEATSVSCFRG
NOV36d PGKYLDQGLDDNYCRNPDGSERPWCYTTDPQIEREFCDLPRCGSEAQPRQEATSVSCFRG
NOV36e PGKFLDQGLDDNYCRNPDGSERPWCYTTDPQIEREFCDLPRCGSEAQPRQEATTVSCFRG
NOV36f PGRFLDQGLDDNYCRNPDGSERPWCYTTDPQIEREFCDLPRCGSEAQPRQEATSVSCFRG
NOV36a KGEGYRGTANTTTAGVPCQRWDAQIPHQRRFTPEKYACKDLRENFCRNPDGSEAPWCFTL
NOV36b KGEGYRGTANTTTAGVPCQRWDAQIPHQHRFTPEKYACKDLRENFCRNPDGSEAPWCFTL
NOV36c KGEGYRGTANTTTAGVPCQRWDAQIPHQHRFTPEKYACKDLRENFCRNPDGSEAPWCFTL
NOV36d KGEGYRGTANTTTAGVPCQRWDAQIPHQHRFTPEKYACKDLRENFCRNPDGSEAPWCFTL
NOV36e KGEGYRGTA1TTTAGVPCQRWDAQIPHQHRFTPEKYACKDLRENFCRNPDGSEAPWCFTL
NOV36f KGEGYRGTANTTTAGVPCQRWDAQIPHQHRFTPEKYACKDLRENFCRNLDGSEAPWCFTL
NOV36a RPGMRAAFCYQIRRCTDDVRPQDCYHGAGEQYRGTVSKTRKGVQCQRWSAETPHKPQFTF
NOV36b RPGMRVGFCYQIRRCTDDVRPQDCYHGAGEQYRGTVSKTRKGVQCQRGSABTPHKPQFTF
NOV36c RPGMRVGFCYQIRRCTDDVRPQGCYHGAGEQYRGTVSKTRKGVQCQRASAETPHKPQFTF
NOV36d RPGMRVGFCYQIRRCTDDVRPQDCYHGAGEQYRGTVSKTRKGVQCQRASAETPHKPQFTF
NOV36e RPGMRAAFCYQIRRCTDDVRPQ-----G-EQYRGTVSKTRKGVQCQRWSAETPHKPQFTF
NOV36f RPGMRVGFCYQIRRCTDDVRPQDCYHGAGEQYRGTVSKTRKGVQCQRASAETPHKPQFTF
NOV36a TSEPHAQLEENFCRNPDGDSHGPWCYTMDPRTPFDYCALRRCADDQPPSILDPP-DQVQF
NOV36b TSEPHAQLEENFCQDPDGDSHGPWCYTMDPRTPFDYCALRRCADDQPPSILDPP-DQVQF
NOV36c TSEPHAQLEENFCRDPDGDSYGPWCYTMDPRTPFDYCALRRCADDQPPSILDPP-DQVQF
NOV36d TSEPHAQLEENFCQDPDGDSHGPWCYTMDPRTPFDYCALRRCADDQPPSILDPPTDQVQF
NOV36e TSEPHAQLEENFCRNPDGDSHGPWCYTMDPRTPFDYCALRRC------------------
NOV36f TSEPHAQLEENFCQTPDGDSHGPWCYTMDPRTPFDYCALRRCADDQPPSILDPP-DQVQF
NOV36a EKCGKRVDRLDQRRSKLRVVGGHPGNSPWTVSLRNR-QGQHFCGGSLVKEQWILTARQCF
NOV36b EKCGKRVDRLDORCSKLRVAGGHPGNSPWTVSLRNR-QGQHFCGGSLVKEQWILTARQCF
NOV36c EKCGKRVDRLDQRCSKLRVAGGHPGNSPWTVSLRNR-QGQHFCGGSLVKEQWILTARQCF
NOV36d EKCGKRVDRLDQRRSKLRVAGGHPGNSPWTVSLGNRRQGQHFCGGSLVKEQWILTARQCF
NOV36e ------------------------------------------------------------
NOV36f EKCGKRVDRLDQRRSKLRVAGGHPGNSPWTVSLGNR-QGQHFCGGSLVKEQWILTARQCF
NOV36a SSCHMPLTGYEVWLGTLFQNPQHGEPSLQRVPVAKMVCGPSGSQLVLLKLERSVTLNQRV
NOV36b SSSHMPLTGYEVWLGTLFQNPQHGEPGLQRVPVAKMLCGPSGSQLVLLKLERSVTLNQRV
NOV36c SSSHMPLTGYEVWLGTLFQNPQHGEPGLQRVPVAKMLCGPSGSQLVLLKLERSVTLNQRV
NOV36d SS-HMPLTGYEVWLGTLFQNPQHGEPGLQRVPVAKMLCGPSGSQLVLLKLERSVTLNQRV
NOV36e ------------------------------------------------------------
NOV36f SSQHMPLTGYEVWLGTLFQNPQHGEPGLQRVPVAKMLCGPSGSQLVLLKLERSVTLNQRV
NOV36a ALICLPPEWYVVPPGTKCEG----------------------------------------
NOV36b ALICLPPE----------------------------------------------------
NOV36c ALICLPPEWYVVPPGTKCEIAGRGETKGTGNDTVLNVALLNVISNQECNIKHRGHVRESE
NOV36d ALICLPPE----------------------------------------------------
NOV36e ------------------------------------------------------------
NOV36f ALICLPPE----------------------------------------------------
NOV36a ----------------DYGGPLACFTHNCWVLEGIIIPNRVCARSRWPAVFTRVSVFVDW
NOV36b ------------------------------------------------------------
NOV36c MCTEGLLAPVGACEGGDYGGPLACFTHNCWVLEGIRIPNRVCARSRWPAVFTRVSVFVDW
NOV36d ------------------------------------------------------------
NOV36e ------------------------------------------------------------
NOV36f ------------------------------------------------------------
NOV36a IHKVMRLG
NOV36b --------
NOV36c IHKVMRLG
NOV36d --------
NOV36e --------
NOV36f --------
NOV36a (SEQ ID NO: 446)
NOV36b (SEQ ID NO: 448)
NOV36c (SEQ ID NO: 450)
NOV36d (SEQ ID NO: 452)
NOV36e (SEQ ID NO: 454)
NOV36f (SEQ ID NO: 456)

Further analysis of the NOV36a protein yielded the following properties shown in Table 36C.

TABLE 36C
Protein Sequence Properties NOV36a

| | |
|---|---|
| SignalP analysis: | Cleavage site between residues 30 and 31 |

PSORT II analysis:
PSG: a new signal peptide prediction method

N-region: length 0; pos.chg 0; neg.chg 0
H-region: length 12; peak value 9.09
PSG score: 4.69

GvH: von Heijne's method for signal seq. recognition

GvH score (threshold: −2.1): 6.16
possible cleavage site: between 29 and 30

>>> Seems to have a cleavable signal peptide (1 to 29)
ALOM: Klein et al's method for TM region allocation

Init position for calculation: 30
Tentative number of TMS(s) for the threshold 0.5: 0
number of TMS(s) . . . fixed
PERIPHERAL Likelihood = 3.07 (at 623)
ALOM score: 3.07 (number of TMSs: 0)

MTOP: Prediction of membrane topology (Hartmann et al.)

Center position for calculation: 14
Charge difference: −3.0 C(0.0) − N(3.0)
N >= C: N-terminal side will be inside

MITDISC: discrimination of mitochondrial targeting seq

| | | | |
|---|---|---|---|
| R content: | 3 | Hyd Moment(75): | 1.11 |
| Hyd Moment(95): | 1.26 | G content: | 4 |
| D/E content: | 1 | S/T content: | 3 |

Score: −4.52

Gavel: prediction of cleavage sites for mitochondrial preseq

R-2 motif at 44 QRS|PL

NUCDISC: discrimination of nuclear localization signals

pat4: none
pat7: PHTRLRR (3) at 102
bipartite: none
content of basic residues: 11.8%
NLS Score: −0.22

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 55.5

COIL: Lupas's algorithm to detect coiled-coil regions

total: 0 residues

Final Results (k = 9/23):

66.7%: extracellular, including cell wall
22.2%: mitochondrial
11.1%: vacuolar

>> prediction for CG54479-06 is exc (k = 9)

A search of the NOV36a protein against the Geneseq database, a proprietary database that contains sequences published in patents and patent publication, yielded several homologous proteins shown in Table 36D.

TABLE 36D
Geneseq Results for NOV36a

| | | NOV36a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW14270 | Human growth factor L5/3 - | 15 . . . 670 | 637/711 (89%) | 0.0 |
| | Homo sapiens, 711 aa. | 1 . . . 711 | 641/711 (89%) | |
| | [US5606029-A, 25-FEB-1997] | | | |
| AAR66602 | Human L5/3 tumour suppressor | 15 . . . 670 | 637/711 (89%) | 0.0 |
| | protein - Homo sapiens, 711 aa. | 1 . . . 711 | 641/711 (89%) | |
| | [US5315000-A, 24-MAY-1994] | | | |
| AAY31157 | Human macrophage stimulating | 15 . . . 670 | 639/716 (89%) | 0.0 |
| | protein - Homo sapiens, 711 aa. | 1 . . . 711 | 642/716 (89%) | |
| | [US5948892-A, 07-SEP-1999] | | | |
| ABB82662 | Human hepatocyte growth factor- | 15 . . . 670 | 636/711 (89%) | 0.0 |
| | like protein (HGFL) - Homo | 1 . . . 711 | 640/711 (89%) | |
| | sapiens, 711 aa. [WO200283074- | | | |
| | A2, 24-OCT-2002] | | | |
| AAB84520 | Amino acid sequence of human | 15 . . . 670 | 638/716 (89%) | 0.0 |
| | macrophage stimulating protein | 1 . . . 710 | 641/716 (89%) | |
| | (MSP) - Homo sapiens, 710 aa. | | | |
| | [WO200144294-A2, 21-JUN- | | | |
| | 2001] | | | |

In a BLAST search of public sequence databases, the NOV36a protein was found to have homology to the proteins shown in the BLASTP data in Table 36E.

TABLE 36E
Public BLASTP Results for NOV36a

| | | NOV36a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH48330 | Macrophage stimulating 1 | 15 . . . 670 | 637/711 (89%) | 0.0 |
| | (Hepatocyte growth factor-like) - | 1 . . . 711 | 641/711 (89%) | |
| | Homo sapiens (Human), 711 aa. | | | |
| P26927 | Hepatocyte growth factor-like | 15 . . . 670 | 636/711 (89%) | 0.0 |
| | protein precursor (Macrophage | 1 . . . 711 | 640/711 (89%) | |
| | stimulatory protein) (MSP) | | | |
| | (Macrophage stimulating protein) - | | | |
| | Homo sapiens (Human), 711 aa. | | | |
| CAD48698 | Sequence 61 from Patent | 15 . . . 670 | 612/712 (85%) | 0.0 |
| | WO0229058 - Homo sapiens | 1 . . . 712 | 626/712 (86%) | |
| | (Human), 712 aa. | | | |
| CAD48697 | Sequence 59 from Patent | 15 . . . 670 | 611/712 (85%) | 0.0 |
| | WO0229058 - Homo sapiens | 1 . . . 712 | 625/712 (86%) | |
| | (Human), 712 aa. | | | |
| Q13208 | Hepatocyte growth factor-like | 50 . . . 606 | 533/557 (95%) | 0.0 |
| | protein homolog - Homo sapiens | 11 . . . 567 | 543/557 (96%) | |
| | (Human), 567 aa. | | | |

PFam analysis indicates that the NOV36a protein contains the domains shown in the Table 36F.

TABLE 36F
Domain Analysis of NOV36a

| | NOV36a | Identities/ | |
|---|---|---|---|
| | Match | Similarities | |
| Pfam Domain | Region | for the Matched Region | Expect Value |
| PAN | 32 . . . 120 | 25/110 (23%) | 1.3e−16 |
| | | 68/110 (62%) | |
| kringle | 124 . . . 200 | 46/85 (54%) | 3.3e−45 |
| | | 70/85 (82%) | |
| kringle | 205 . . . 282 | 48/85 (56%) | 2e−48 |
| | | 73/85 (86%) | |
| kringle | 297 . . . 375 | 44/85 (52%) | 3.6e−49 |
| | | 73/85 (86%) | |
| kringle | 384 . . . 462 | 44/85 (52%) | 1.5e−50 |
| | | 76/85 (89%) | |
| trypsin | 498 . . . 663 | 73/263 (28%) | 7.3e−21 |
| | | 126/263 (48%) | |

### Example 37

The NOV37 clone was analyzed, and the nucleotide and encoded polypeptide sequences are shown in Table 37A.

TABLE 37A
NOV37 Sequence Analysis

| | | |
|---|---|---|
| NOV37a, CG54539-02 | SEQ ID NO: 457 | 1567 bp |
| DNA Sequence | ORF Start: ATG at 2 | ORF Stop: TGA at 1523 |

TATGGGGTGTTGGGGTCGGAACCGGGGCCGGCTGCTGTGCATGCTGGCGCTGACCTTCATGTTCATGG
TGCTGGAGGTGGTGGTGAGCCGGGTGACCTCGTCGCTGGCGATGCTCTCCGACTCCTTCCACATGCTG
TCGGACGTGCTGGCGCTGGTGGTGGCGCTGGTGGCCGAGCGCTTCGCCCGGCGGACCCACGCCACCCA
GAAGAACACGTTCGGCTGGATCCGAGCCGAGGTAATGGGGGCTCTGGTGAACGCCATCTTCCTGACTG
GCCTCTGTTTCGCCATCCTGCTGGAGGCCATCGAGCGCTTCATCGAGCCGCACGAGATGCAGCAGCCG
CTGGTGGTCCTTGGGGTCGGCGTGGCCGGGCTGCTGGTCAACGTGCTGGGGCTCTGCCTCTTCCACCA
TCACAGCGGCTTCAGCCAGGACTCCGGCCACGGCCACTCGCACGGGGGTCACGGCTACGGCCACGGCC
TCCCCAAGGGGCCTCGCGTTAAGAGCACCCGCCCCGGGAGCAGCGACATCAACGTGGCCCCGGGCGAG
CAGGGTCCCGACCAGGAGGAGACCAACACCCTGGTGGCCAATACCAGCCAACTCAACGGGCTGAAATT
GGACCCCGCAGACCCAGAAAACCCCAGAAGTGGTGATACAGTGGAAGTACAAGTGAATGGAAATCTTG
TCAGAGAACCTGACCATATGGAACTGGAAGAAGATAGGGCTGGACAACTTAACATGCGTGGAGTTTTT
CTGCATGTCCTTGGAGATGCCTTGGGTTCAGTGATTGTAGTAGTAAATGCCTTAGTCTTTTACTTTTC
TTGGAAAGGTTGTTCTGAAGGGGATTTTTGTGTGAATCCATGTTTCCCTGACCCCTGCAAAGCATTTG
TAGAAATAATTAATAGTACTCATGCATCAGTTTATGAGGCTGGTCCTTGCTGGGTGCTATATTTAGAT
CCAACTCTTTGTGTTGTAATGGTTTGTATACTTCTTTACACAACCTATCCATTACTTAAGGAATCTGC
TCTTATTCTTCTACAAACTGTTCCTAAACAAATTGATATCAGAAATTTGATAAAAGAACTTCGAAATG
TTGAAGGAGTTGAGGAAGTTCATGAATTACATGTTTGGCAACTTGCTGGAAGCAGAATCATTGCCACT
GCTCACATAAAATGTGAAGATCCAACATCATACATGGAGGTGGCTAAAACCATTAAAGACGTTTTTCA
TAATCACGGAATTCACGCTACTACCATTCAGCCTGAATTTGCTAGTGTAGGCTCTAAATCAAGTGTAG
TTCCGTGTGAACTTGCCTGCAGAACCCAGTGTGCTTTGAAGCAATGTTGTGGGACACTACCACAAGCC
CCTTCTGGAAAGGATGCAGAAAAGACCCCAGCAGTTAGCATTTCTTGTTTAGAACTTAGTAACAATCT
AGAGAAGAAGCCCAGGAGGACTAAAGCTGAAAACATCCCTGCTGTTGTGATAGAGATTAAAAACATGC
CAAACAAACAACCTGAATCATCTTTGTGAGTCTTGAAAAAGATGTGATATTTGACTTTTGCTTTAAAC
TGC

| | | | |
|---|---|---|---|
| NOV37a, CG54539-02 | SEQ ID NO: 458 | 507 aa | MW at 55325.3kD |
| Protein Sequence | | | |

MGCWGRNRGRLLCMLALTFMFMVLEVVVSRVTSSLAMLSDSFHMLSDVLALVVALVAERFARRTHATQ
KNTFGWIRAEVMGALVNAIFLTGLCFAILLEAIERFIEPHEMQQPLVVLGVGVAGLLVNVLGLCLFHH
HSGFSQDSGHGHSHGGHGYGHGLPKGPRVKSTRPGSSDINVAPGEQGPDQEETNTLVANTSNSNGLKL
DPADPENPRSGDTVEVQVNGNLVREPDHMELEEDRAGQLNMRGVFLHVLGDALGSVIVVVNALVFYFS
WKGCSEGDFCVNPCFPDPCKAFVEIINSTHASVYEAGPCWVLYLDPTLCVVMVCILLYTTYPLLKESA
LILLQTVPKQIDIRNLIKELRNVEGVEEVHELHVWQLAGSRIIATAHIKCEDPTSYMEVAKTIKDVFH
NHGIHATTIQPEFASVGSKSSVVPCELACRTQCALKQCCGTLPQAPSGKDAEKTPAVSISCLELSNNL
EKKPRRTKAENIPAVVIEIKNMPNKQPESSL

| | | |
|---|---|---|
| NOV37b, CG54539-01 | SEQ ID NO: 459 | 1665 bp |
| DNA Sequence | ORF Start: ATG at 77 | ORF Stop: TGA at 1598 |

CGACCCTCCGCGTCCCGCCAACGCCGCCGCTGCACCAGTCTCCGGGCCGGGCTCGGCGGGCCCCGCAG
CCGCAGCCATGGGGTGTTGGGGTCGGAACCGGGGCCGGCTGCTGTGCATGCTGGCGCTGACCTTCATG
TTCATGGTGCTGGAGGTGGTGGTGAGCCGGGTGACCTCGTCGCTGGCGATGCTCTCCGACTCCTTCCA
CATGCTGTCGGACGTGCTGGCGCTGGTGGTGGCGCTGGTGGCCGAGCGCTTCGCCCGGCGGACCCACG
CCACCCAGAAGAACACGTTCGGCTGGATCCGAGCCGAGGTAATGGGGGCTCTGGTGAACGCCATCTTC
CTGACTGGCCTCTGTTTCGCCATCCTGCTGGAGGCCATCGAGCGCTTCATCGAGCCGCACGAGATGCA
GCAGCCGCTGGTGGTCCTTGGGGTCGGCGTGGCCGGGCTGCTGGTCAACGTGCTGGGGCTCTGCCTCT
TCCACCATCACAGCGGCTTCAGCCAGGACTCCGGCCACGGCCACTCGCACGGGGGTCACGGCCACGGC
CACGGCCTCCCCAAGGGGCCTCGCGTTAAGAGCACCCGCCCCGGGAGCAGCGACATCAACGTGGCCCC
GGGCGAGCAGGGTCCCGACCAGGAGGAGACCAACACCCTGGTGGCCAATACCAGCAACTCCAACGGGC
TGAAATTGGACCCCGCGGACCCAGAAAACCCCAGAAGTGGTGATACAGTGGAAGTACAAGTGAATGGA
AATCTTGTCAGAGAACCTGACCATATGGAACTGGAAGAAGATAGGGCTGGACAACTTAACATGCGTGG
AGTTTTTCTGCATGTCCTTGGAGATGCCTTGGGTTCAGTGATTGTAGTAGTAAATGCCTTAGTCTTTT
ACTTTTCTTGGAAAGGTTGTTCTGAAGGGGATTTTTGTGTGAATCCATGTTTCCCTGACCCCTGCAAA
GCATTTGTAGAAATAATTAATAGTACTCATGCATCAGTTTATGAGGCTGGTCCTTGCTGGGTGCTATA
TTTAGATCCAACTCTTTGTGTTGTAATGGTTTGTATACTTCTTTACACAACCTATCCATTACTTAAGG
AATCTGCTCTTATTCTTCTACAAACTGTTCCTAAACAAATTGATATCAGAAATTTGATAAAAGAACTT
CGAAATGTTGAAGGAGTTGAGGAAGTTCATGAATTACATGTTTGGCAACTTGCTGGAAGCAGAATCAT
TGCCACTGCTCACATAAAATGTGAAGATCCAACATCATACATGGAGGTGGCTAAAACCATTAAAGACG
TTTTTCATAATCACGGAATTCACGCTACTACCATTCAGCCTGAATTTGCTAGTGTAGGCTCTAAATCA
AGTGTAGTTCCGTGTGAACTTGCCTGCAGAACCCAGTGTGCTTTGAAGCAATGTTGTGGGACACTACC
ACAAGCCCCTTCTGGAAAGGATGCAGAAAAGACCCCAGCAGTTAGCATTTCTTGTTTAGAACTTAGTA
ACAATCTAGAGAAGAAGCCCAGGAGGACTAAAGCTGAAAACATCCCTGCTGTTGTGATAGAGATTAAA
AACATGCCAAACAAACAACCTGAATCATCTTTGTGAGTCTTGAAAAAGATGTGATATTTGACTTTTGC
TTTAAACTGCAAGAGGAAAAAGACTCCACTGAA

| | | | |
|---|---|---|---|
| NOV37b, CG54539-01 | SEQ ID NO: 460 | 507 aa | MW at 55299.3kD |
| Protein Sequence | | | |

MGCWGRNRGRLLCMLALTFMFMVLEVVVSRVTSSLAMLSDSFHMLSDVLALVVALVAERFARRTHATQ
KNTFGWIRAEVMGALVNAIFLTGLCFAILLEAIERFIEPHEMQQPLVVLGVGVAGLLVNVLGLCLFHH
HSGFSQDSGHGHSHGGHGHGHGLPKGPRVKSTRPGSSDINVAPGEQGPDQEETNTLVANTSNSNGLKL
DPADPENPRSGDTVEVQVNGNLVREPDHMELEEDRAGQLNMRGVFLHVLGDALGSVIVVVNALVFYFS
WKGCSEGDFCVNPCFPDPCKAFVEIINSTHASVYEAGPCWVLYLDPTLCVVMVCILLYTTYPLLKESA
LILLQTVPKQIDIRNLIKELRNVEGVEEVHELHVWQLAGSRIIATAHIKCEDPTSYMEVAKTIKDVFH
NHGIHATTIQPEFASVGSKSSVVPCELACRTQCALKQCCGTLPQAPSGKDAEKTPAVSISCLELSNNL
EKKPRRTKAENIPAVVIEIKNMPNKQPESSL

| | | |
|---|---|---|
| NOV37c, SNP13382438 of | SEQ ID NO: 461 | 1567 bp |
| CG54539-02, DNA Sequence | ORF Start: ATG at 2 | ORF Stop: TGA at 1523 |
| | SNP Pos: 464 | SNP Change: T to C |

TATGGGGTGTTGGGGTCGGAACCGGGGCCGGCTGCTGTGCATGCTGGCGCTGACCTTCATGTTCATGG
TGCTGGAGGTGGTGGTGAGCCGGGTGACCTCGTCGCTGGCGATGCTCTCCGACTCCTTCCACATGCTG
TCGGACGTGCTGGCGCTGGTGGTGGCGCTGGTGGCCGAGCGCTTCGCCCGGCGGACCCACGCCACCCA
GAAGAACACGTTCGGCTGGATCCGAGCCGAGGTAATGGGGGCTCTGGTGAACGCCATCTTCCTGACTG
GCCTCTGTTTCGCCATCCTGCTGGAGGCCATCGAGCGCTTCATCGAGCCGCACGAGATGCAGCAGCCG
CTGGTGGTCCTTGGGGTCGGCGTGGCCGGGCTGCTGGTCAACGTGCTGGGGCTCTGCCTCTTCCACCA
TCACAGCGGCTTCAGCCAGGACTCCGGCCACGGCCACTCGCACGGGGGTCACGGCCACGGCCACGGCC
TCCCCAAGGGGCCTCGCGTTAAGAGCACCCGCCCCGGGAGCAGCGACATCAACGTGGCCCCGGGCGAG
CAGGGTCCCGACCAGGAGGAGACCAACACCCTGGTGGCCAATACCAGCAACTCCAACGGGCTGAAATT
GGACCCCGCAGACCCAGAAAACCCCAGAAGTGGTGATACAGTGGAAGTACAAGTGAATGGAAATCTTG
TCAGAGAACCTGACCATATGGAACTGGAAGAAGATAGGGCTGGACAACTTAACATGCGTGGAGTTTTT
CTGCATGTCCTTGGAGATGCCTTGGGTTCAGTGATTGTAGTAGTAAATGCCTTAGTCTTTTACTTTTC
TTGGAAAGGTTGTTCTGAAGGGGATTTTTGTGTGAATCCATGTTTCCCTGACCCCTGCAAAGCATTTG
TAGAAATAATTAATAGTACTCATGCATCAGTTTATGAGGCTGGTCCTTGCTGGGTGCTATATTTAGAT
CCAACTCTTTGTGTTGTAATGGTTTGTATACTTCTTTACACAACCTATCCATTACTTAAGGAATCTGC
TCTTATTCTTCTACAAACTGTTCCTAAACAAATTGATATCAGAAATTTGATAAAAGAACTTCGAAATG
TTGAAGGAGTTGAGGAAGTTCATGAATTACATGTTTGGCAACTTGCTGGAAGCAGAATCATTGCCACT
GCTCACATAAAATGTGAAGATCCAACATCATACATGGAGGTGGCTAAAACCATTAAAGACGTTTTTCA
TAATCACGGAATTCACGCTACTACCATTCAGCCTGAATTTGCTAGTGTAGGCTCTAAATCAAGTGTAG
TTCCGTGTGAACTTGCCTGCAGAACCCAGTGTGCTTTGAAGCAATGTTGTGGGACACTACCACAAGCC
CCTTCTGGAAAGGATGCAGAAAAGACCCCAGCAGTTAGCATTTCTTGTTTAGAACTTAGTAACAATCT
AGAGAAGAAGCCCAGGAGGACTAAAGCTGAAAACATCCCTGCTGTTGTGATAGAGATTAAAAACATGC
CAAACAAACAACCTGAATCATCTTTGTGAGTCTTGAAAAAGATGTGATATTTGACTTTTGCTTTAAAC
TGC

| | | | |
|---|---|---|---|
| NOV37c, SNP13382438 of | SEQ ID NO: 462 | 507 aa | MW at 55299.3kD |
| CG54539-02, Protein | SNP Pos: 155 | | SNP Change: Tyr to His |
| Sequence | | | |

MGCWGRNRGRLLCMLALTFMFMVLEVVVSRVTSSLAMLSDSFHMLSDVLALVVALVAERFARRTHATQ
KNTFGWIRAEVMGALVNAIFLTGLCFAILLEAIERFIEPHEMQQPLVVLGVGVAGLLVNVLGLCLFHH
HSGFSQDSGHGHSHGGHGHGHGLPKGPRVKSTRPGSSDINVAPGEQGPDQEETNTLVANTSNSNGLKL
DPADPENPRSGDTVEVQVNGNLVREPDHMELEEDRAGQLNMRGVFLHVLGDALGSVIVVVNALVFYFS
WKGCSEGDFCVNPCFPDPCKAFVEIINSTHASVYEAGPCWVLYLDPTLCVVMVCILLYTTYPLLKESA
LILLQTVPKQIDIRNLIKELRNVEGVEEVHELHVWQLAGSRIIATAHIKCEDPTSYMEVAKTIKDVFH
NHGIHATTIQPEFASVGSKSSVVPCELACRTQCALKQCCGTLPQAPSGKDAEKTPAVSISCLELSNNL
EKKPRRTKAENIPAVVIEIKNNPNKQPESSL

A ClustalW comparison of the above protein sequences yields the following sequence alignment shown in Table 37B.

TABLE 37B
Comparison of the NOV37 protein sequences.

NOV37a MGCWGRNRGRLLCMLALTFMFMVLEVVVSRVTSSLAMLSDSFHMLSDVLALVVALVAERF
NOV37b MGCWGRNRGRLLCMLALTFMFMVLEVVVSRVTSSLAMLSDSFHMLSDVLALVVALVAERF
NOV37a ARRTHATQKNTFGWIRAEVMGALVNAIFLTGLCFAILLEAIERFIEPHEMQQPLVVLGVG
NOV37b ARRTHATQKNTFGWIRAEVMGALVNAIFLTGLCFAILLEAIERFIEPHEMQQPLVVLGVG
NOV37a VAGLLVNVLGLCLFHHHSGFSQDSGHGHSHGGHGYGHGLPKGPRVKSTRPGSSDINVAPG
NOV37b VAGLLVNVLGLCLFHHHSGFSQDSGHGHSHGGHGHGHGLPKGPRVKSTRPGSSDINVAPG
NOV37a EQGPDQEETNTLVAITSNSNGLKLDPADPENPRSGDTVEVQVNGNLVREPDHMELEEDRA
NOV37b EQGPDQEETNTLVANTSNSNGLKLDPADPENPRSGDTVEVQVNGNLVREPDHMELEEDRA
NOV37a GQLNMRGVFLHVLGDALGSVIVVVNALVFYFSWKGCSEGDFCVNPCFPDPCKAFVEIINS
NOV37b GQLNMRGVFLHVLGDALGSVIVVVNALVFYFSWKGCSEGDFCVNPCFPDPCKAFVEIINS
NOV37a THASVYEAGPCWVLYLDPTLCVVMVCILLYTTYPLLKESALILLQTVPKQIDIRNLIKEL
NOV37b THASVYEAGPCWVLYLDPTLCVVMVCILLYTTYPLLKESALILLQTVPKQIDIRNLIKEL
NOV37a RNVEGVEEVHELHVWQLAGSRIIATAHIKCEDPTSYMEVAKTIKDVFHNHGIHATTIQPE
NOV37b RNVEGVEEVHELHVWQLAGSRIIATAHIKCEDPTSYMEVAKTIKDVFHNHGIHATTIQPE
NOV37a FASVGSKSSVVPCELACRTQCALKQCCGTLPQAPSGKDAEKTPAVSISCLELSNNLEKKP
NOV37b FASVGSKSSVVPCELACRTQCALKQCCGTLPQAPSGKDAEKTPAVSISCLELSNNLEKKP
NOV37a RRTKAENIPAVVIEIKNMPNKQPESSL
NOV37b RRTKAENIPAVVIEIKNMPNKQPESSL
NOV37a (SEQ ID NO: 458)
NOV37b (SEQ ID NO: 460)

Further analysis of the NOV37a protein yielded the following properties shown in Table 37C.

TABLE 37C
Protein Sequence Properties NOV37a

| | |
|---|---|
| SignalP analysis: | Cleavage site between residues 30 and 31 |

PSORT II analysis:
PSG: a new signal peptide prediction method

N-region: length 10; pos.chg 3; neg.chg 0
H-region: length 14; peak value 12.34
PSG score: 7.94

GvH: von Heijne's method for signal seq. recognition

GvH score (threshold: −2.1): −2.29
possible cleavage site: between 29 and 30

>>> Seems to have no N-terminal signal peptide
ALOM: Klein et al's method for TM region allocation

| Init position for calculation: 1 | | |
|---|---|---|
| Tentative number of TMS(s) for the threshold 0.5: 6 | | |
| INTEGRAL | Likelihood = −8.76 | Transmembrane 12-28 |
| INTEGRAL | Likelihood = −3.66 | Transmembrane 41-57 |
| INTEGRAL | Likelihood = −7.54 | Transmembrane 82-98 |
| INTEGRAL | Likelihood = −7.43 | Transmembrane 114-130 |
| INTEGRAL | Likelihood = −5.57 | Transmembrane 248-264 |
| INTEGRAL | Likelihood = −7.01 | Transmembrane 313-329 |

| | |
|---|---|
| PERIPHERAL | Likelihood = 6.21 (at 421) |

ALOM score: −8.76 (number of TMSs: 6)

MTOP: Prediction of membrane topology (Hartmann et al.)

Center position for calculation: 19
Charge difference: −4.0 C(0.0) − N(4.0)
N >= C: N-terminal side will be inside

>>> membrane topology: type 3a
MITDISC: discrimination of mitochondrial targeting seq

| | | | |
|---|---|---|---|
| R content: | 3 | Hyd Moment(75): | 5.99 |
| Hyd Moment(95): | 2.66 | G content: | 3 |
| D/E content: | 1 | S/T content: | 1 |

Score: −3.81

Gavel: prediction of cleavage sites for mitochondrial preseq

R-2 motif at 40 SRV|TS

NUCDISC: discrimination of nuclear localization signals

pat4: KKPR (4) at 478
pat4: KPRR (4) at 479
pat7: PRRTKAE (5) at 480
bipartite: none
content of basic residues: 8.3%
NLS Score: 0.46

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

66.7%: endoplasmic reticulum
22.2%: mitochondrial
11.1%: nuclear

>> prediction for CG54539-02 is end (k = 9)

A search of the NOV37a protein against the Geneseq database, a proprietary database that contains sequences published in patents and patent publication, yielded several homologous proteins shown in Table 37D.

TABLE 37D
Geneseq Results for NOV37a

| | | NOV37a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG73033 | Human transporter polypeptide - | 1 . . . 507 | 506/507 (99%) | 0.0 |
| | Homo sapiens, 507 aa. | 1 . . . 507 | 507/507 (99%) | |
| | [US2002106721-A1, 08-AUG- | | | |
| | 2002] | | | |
| ABG73033 | Human transporter polypeptide - | 1 . . . 507 | 506/507 (99%) | 0.0 |
| | Homo sapiens, 507 aa. | 1 . . . 507 | 507/507 (99%) | |
| | [US2002106721-A1, 08-AUG- | | | |
| | 2002] | | | |
| AAG67549 | Amino acid sequence of a human | 1 . . . 507 | 504/507 (99%) | 0.0 |
| | transporter protein - Homo | 1 . . . 507 | 506/507 (99%) | |
| | sapiens, 507 aa. [WO200164878- | | | |
| | A2, 07-SEP-2001] | | | |
| AAE16348 | Human zinc transporter like | 1 . . . 507 | 506/512 (98%) | 0.0 |
| | protein, POLY12 - Homo sapiens, | 1 . . . 512 | 507/512 (98%) | |
| | 512 aa. [WO200185767-A2, 15- | | | |
| | NOV-2001] | | | |
| AAY86241 | Human secreted protein | 14 . . . 501 | 482/488 (98%) | 0.0 |
| | HOABR60, SEQ ID NO: 156 - | 1 . . . 488 | 484/488 (98%) | |
| | Homo sapiens, 490 aa. | | | |
| | [WO9966041-A1, 23-DEC-1999] | | | |

In a BLAST search of public sequence databases, the NOV37a protein was found to have homology to the proteins shown in the BLASTP data in Table 37E.

TABLE 37E
Public BLASTP Results for NOV37a

| | | NOV37a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9Y6M5 | Zinc transporter 1 (ZnT-1) - | 1 . . . 507 | 505/507 (99%) | 0.0 |
| | Homo sapiens (Human), 507 aa. | 1 . . . 507 | 506/507 (99%) | |
| Q62720 | Zinc transporter 1 (ZnT-1) - | 1 . . . 507 | 437/511 (85%) | 0.0 |
| | Rattus norvegicus (Rat), 507 aa. | 1 . . . 507 | 468/511 (91%) | |
| Q60738 | Zinc transporter 1 (ZnT-1) - Mus | 1 . . . 507 | 431/509 (84%) | 0.0 |
| | musculus (Mouse), 503 aa. | 1 . . . 503 | 464/509 (90%) | |
| AAH46675 | Similar to solute carrier family 30 | 4 . . . 500 | 297/508 (58%) | e−156 |
| | (zinc transporter), member 1 - | 2 . . . 493 | 366/508 (71%) | |
| | Xenopus laevis (African clawed | | | |
| | frog), 494 aa. | | | |
| CAD58282 | Sequence 16 from Patent | 1 . . . 485 | 183/503 (36%) | 2e−69 |
| | WO0240656 - Homo sapiens | 1 . . . 454 | 254/503 (50%) | |
| | (Human), 485 aa. | | | |

PFam analysis indicates that the NOV37a protein contains the domains shown in the Table 37F.

TABLE 37F
Domain Analysis of NOV37a

| | NOV37a | Identities/ | |
|---|---|---|---|
| | Match | Similarities | |
| Pfam Domain | Region | for the Matched Region | Expect Value |
| Cation_efflux | 11 . . . 426 | 102/435 (23%) | 4.8e−56 |
| | | 323/435 (74%) | |

### Example 38

The NOV38 clone was analyzed, and the nucleotide and encoded polypeptide sequences are shown in Table 38A.

TABLE 38A
NOV38 Sequence Analysis

| | | |
|---|---|---|
| NOV38a, CG54683-05 | SEQ ID NO: 463 | 1416 bp |
| DNA Sequence | ORF Start: ATG at 4 | ORF Stop: TGA at 1402 |

GAGATGGTCCTGGCTTTCCAGTTAGTCTCCTTCACCTACATCTGGATCATATTGAAACCAAATGTTTG
TGCTGCTTCTAACATCAAGATGACACACCAGCGGTGCTCCTCTTCAATGAAACAAACCCAAGAAACTA
GAATGAAGAAAGATGACAGTACCAAAGCGCGGCCTCAGAAATATGAGCAACTTCTCCATATAGAGGAC
AACGATTTCGCAATGAGACCTGGATTTGGAGGGTCTCCAGTGCCAGTAGGTATAGATGTCCATGTTGA
AAGCATTGACAGCATTTCAGAGACTAACATGGTAGACTTTACAATGACTTTTTATCTCAGGCATTACT
GGAAAGACGAGAGGCTCTCCTTTCCTAGCACAGCAAACAAAAGCATGACATTTGATCATAGATTGACC
AGAAAGATCTGGGTGCCTGATATCTTTTTTGTCCACTCTAAAAGATCCTTCATCCATGATACAACTAT
GGAGAATATCATGCTGCGCGTACACCCTGATGGAAACGTCCTCCTAAGTCTCAGGATAACGGTTTCGG
CCATGTGCTTTATGGATTTCAGCAGGTTTCCTCTTGACACTCAAAATTGTTCTCTTGAACTGGAAAGC
GCCTACAATGAGGATGACCTAATGCTATACTGGAAACACGGAAACAAGTCCTTAAATACTGAAGAACA
TATGTCCCTTTCTCAGTTCTTCATTGAAGACTTCAGTGCATCTAGTGGATTAGCTTTCTATAGCAGCA
CAGGTTGGTACAATAGGCTTTTCATCAACTTTGTGCTAAGGAGGCATGTTTTCTTCTTTGTGCTGCAA
ACCTATTTCCCAGCCATATTGATGGTGATGCTTTCATGGGTTTCATTTTGGATTGACCGAAGAGCTGT
TCCTGCAAGAGTTTCCCTGGGTGGAATCACCACAGTGCTGACCATGTCCACAATCATCACTGCTGTGA
GCGCCTCCATGCCCCAGGTGTCCTACCTCAAGGCTGTGGATGTGTACCTGTGGGTCAGCTCCCTCTTT
GTGTTCCTGTCAGTCATTGAGTATGCAGCTGTGAACTACCTCACCACAGTGGAAGAGCGGAAACAATT
CAAGAAGACAGGAAAGATTTCTAGGATGTACAATATTGATGCAGTTCAAGCTATGGCCTTTGATGGTT
GTTACCATGACAGCGAGATTGACATGGACCAGACTTCCCTCTCTCTAAACTCAGAAGACTTCATGAGA
AGAAAATCGATATGCAGCCCCAGCACCGATTCATCTCGGATAAAGAGAAGAAAATCCCTAGGAGGACA
TGTTGGTAGAATCATTCTGGAAAACAACCATGTCATTGACACCTATTCTAGGATTNTATTCCCCATTG
TGTATATCTTATTTAATTTGTTTTACTGGGGTGTATATGTATGAAGGGGAATTTCA

| | | | |
|---|---|---|---|
| NOV38a, CG54683-05 | SEQ ID NO: 464 | 466 aa | MW at 53919.5kD |
| Protein Sequence | | | |

MVLAFQLVSFTYIWIILKPNVCAASNIKMTHQRCSSSMKQTQETRMKKDDSTKARPQKYEQLLHIEDN
DFAMRPGFGGSPVPVGIDVHVESIDSISETNMVDFTMTFYLRHYWKDERLSFPSTANKSMTFDHRLTR
KIWVPDIFFVHSKRSFIHDTTMENINLRVHPDGNVLLSLRITVSAMCFMDFSRFPLDTQNCSLELESA
YNEDDLMLYWKHGNKSLNTEEHMSLSQFFIEDFSASSGLAFYSSTGWYNRLFINFVLRRHVFFFVLQT
YFPAILMVMLSWVSFWIDRRAVPARVSLGGITTVLTMSTIITAVSASMPQVSYLKAVDVYLWVSSLFV
FLSVIEYAAVNYLTTVEERKQFKKTGKISRMYNIDAVQAMAFDGCYHDSEIDMDQTSLSLNSEDFMRR
KSICSPSTDSSRIKRRKSLGGHVGRIILENNHVIDTYSRIXFPIVYILFNLFYWGVYV

| | | |
|---|---|---|
| NOV38b, CG54683-01 | SEQ ID NO: 465 | 1875 bp |
| DNA Sequence | ORF Start: ATG at 10 | ORF Stop: TGA at 1411 |

TTGGAAGAGATGGTCCTGGCTTTCCAGTTAGTCTCCTTCACCTACATCTGGATCATATTGAAACCAAA
TGTTTGTGCTGCTTCTAACATCAAGATGACACACCAGCGGTGCTCCTCTTCAATGAAACAAACCTGGA
TGCAAGAAACTAGAATGAAGAAAGATGACAGTACCAAAGCGCGGCCTCAGAAATATGAGCAACTTCTC
CATATAGAGGACAACGATTTCGCAATGAGACCTGGATTTGGAGGTTCTCCAGTGCCAGTAGGTATAGA
TGTCCATGTTGAAAGCATTGACAGCATTTCAGAGACTAACATGGACTTTACAATGACTTTTTATCTCA
GGCATTACTGGAAAGACGAGAGGCTCTCCTTTCCTAGCACAGCAAACAAAAGCATGACATTTGATCAT
AGACACTTGCGGTATTCGTTATTCATCAGAAGGCTGTATCTGTTATACTGCCAGAGGTCTTTCTTCTC
ACCCTCATCCATACTTCCCTCATCTCCAGACATCCATGCACCTGGTACATCTAAAAGCAGTTTGTCTG
ATAGCCTTGTATGTATATCTGAAAAAAACTTGCCAGGACACAGTAAAAACACACCTCTTGCAATGTCA
GATGTAGCCTACAATGAGGATGACCTAATGCTATACTGGAAACACGGAAACAAGTCCTTAAATACTGA
AGAACATATGTCCCTTTCTCAGTTCTTCATTGAAGACTTCAGTGCATCTAGTGGATTAGCTTTCTATA
GCAGCACAGGTACAGCATTTTACATGGGTGATTCATCAGCATTTATTGGACATCTACTGTTTTTGATC
TGGAGTTCCAGGAAAAGACCAGGTTTAGAGATGTTGGGTTTGGGAATTCTCAGAATCTGGGTAATAAC
TAGAGCCATGGATAAGAAAATGGAAATGGGAATCACCACAGTGCTGACCATGTCCACAATCATCACTG
CTGTGAGCGCCTCCATGCCCCAGGTGTCCTACCTCAAGGCTGTGGATGTGTACCTGTGGGTCAGCTCC
CTCTTTGTGTTCCTGTCAGTCATTGAGTATGCAGCTGTGAACTACCTCACCACAGTGGAAGAGCGGAA
ACAATTCAAAAAAAGTTTTTCAAAGATTTCTAGGATGTACAATATTGATGCAGTTCAAGCTATGGCCT
TTGATGGTTGTTACCATGACAGCGAGATTGACATGGACCAGACTTCCCTCTCTCTAAACTCAGAAGAC
TTCATGAGAAGAAAATCGATATGCAGCCCCAGCACCGATTCATCTCGGATAAAGAGAAGAAAATCCCT
AGGAGGACATGTTGGTAGAATCATTCTGGAAAACAACCATGTCATTGACACCTATTCTAGGATTTTAT
TCCCCATTGTGTATATCTTTTTTAATTTGTTTTACTGGGGTGTATATGTATGAAGGGGAATTTCAAAT
GTATACAACTTTAAAGCCAGATGATGTTTAAAAACAAAACTCTTGAATATGAGTTGGATAGTCCTAGA
TGGAACTGGGAAAGAGCAAGTCACCTCTCCTGCCCTAATGAAAATTTGAAAGCTGTCTGATTTACATC
TAAGAAAGAGTTTAGGTCCTAGAAAAGTTTGACTCCATAAATAAGAGTCATAGGCATGTGTATTATGG
GAAAAACAGTTTTCCATTGGGAAGGGCTTTATAACTACTTCATCTGAACCCTCCTTCTTTCTTAATGA
AATGTTCTTTATTTAACTAGGGAAGAAAGCTGGACTATAACAATAATTCAAAGATATTTTGTTTCTTA
GTGCCAGCCAAGTGCCTGGTTATCTACCAGAGCTCAACCGTCCTAGGCAAGAACATCCACATAGAGGT
GGTATCATCCACACTCACACAGCTGAGAATCCTATGAAG

| | | | |
|---|---|---|---|
| NOV38b, CG54683-01 | SEQ ID NO: 466 | 467 aa | MW at 53522.9kD |
| Protein Sequence | | | |

MVLAFQLVSFTYIWIILKPNVCAASNIKMTHQRCSSSMKQTWMQETRMKKDDSTKARPQKYEQLLHIE
DNDFAMRPGFGGSPVPVGIDVHVESIDSISETNMDFTMTFYLRHYWKDERLSFPSTANKSMTFDHRHL
RYSLFIRRLYLLYCQRSFFSPSSILPSSPDIHAPGTSKSSLSDSLVCISEKNLPGHSKNTPLAMSDVA
YNEDDLMLYWKHGNKSLNTEEHMSLSQFFIEDFSASSGLAFYSSTGTAFYMGDSSAFIGHLLFLIWSS
RKRPGLEMLGLGILRIWVITRAMDKKMEMGITTVLTMSTIITAVSASMPQVSYLKAVDVYLWVSSLFV
FLSVIEYAAVNYLTTVEERKQFKKSFSKISRMYNIDAVQAMAFDGCYHDSEIDMDQTSLSLNSEDFMR
RKSICSPSTDSSRIKRRKSLGGHVGRIILENNHVIDTYSRILFPIVYIFFNLFYWGVYV

| | | |
|---|---|---|
| NOV38c, GG54683-02 | SEQ ID NO: 467 | 1444 bp |
| DNA Sequence | ORF Start: ATG at 21 | ORF Stop: TGA at 1425 |

GTTTTTTTGTTTTGGAAGAGATGGTCCTGGCTTTCCAGTTAGTCTCCTTCACCTACATCTGGATCATA
TTGAAACCAAATGTTTGTGCTGCTTCTAACATCAAGATGACACACCAGCGGTGCTCCTCTTCAATGAA
ACAAACCTGCAAACAAGAAACTAGAATGAAGAAAGATGACAGTACCAAAGCGCGGCCTCAGAAATATG
AGCAACTTCTCCATATAGAGGACAACGATTTCGCAATGAGACCTGGATTTGGAGGGTCTCCAGTGCCA
GTAGGTATAGATGCCCATGTTGAAAGCATTGACAGCATTTCAGAGACTAACATGGACTTTACAATGAC
TTTTTATCTCAGGCATTACTGGAAAGACGAGAGGCTCTCCTTTCCTAGCACAGCAAACAAAAGCATGA
CATTTGATCATAGATTGACCAGAAAGATCTGGGTGCCTGATATCTTTTTTGTCCACTCTAAAAGATCC
TTCATCCATGATACAACTATGGAGAATATCATGCTGCGCGTACACCCTGATGGAAACGTCCTCCTAAG
TCTCAGGATAACGGTTTCGGCCATGTGCTTTATGGATTTCAGCAGGTTTCCTCTTGACGACACTCAAA
ATTGTTCTCTTGAACTGGAAAGCTGTGCCTACAATGAGGATGACCTAATGCTATACTGGAAACACGGA
AACAAGTCCTTAAATACTGAAGAACATATGTCCCTTTCTCAGTTCTTCATTGAAGACTTCAGTGCATC
TAGTGGATTAGCTTTCTATAGCAGCACAGGTTGGTACAATAGGCTTTTCATCAACTTTGTGCTAAGGA
GGCATGTTTTCTTCTTTGTGCTGCAAACCTATTTCCCAGCCATATTGATGGTGATGCTTTCATGGGTT
TCATTTTGGATTGACCGAAGAGCTGTTCCTGCAAGAGTTTCCCTGGGTATCACCACAGTGCTGACCAT
GTCCACAATCATCACTGCTGTGAGCGCCTCCATGCCCCAGGTGTCCTACCTCAAGGCTGTGGATGTGT
ACCTGTGGGTCAGCTCCCTCTTTGTGTTCCTGTCAGTCATTGAGTATGCAGCTGTGAACTACCTCACC
ACAGTGGAAGAGCGGAAACAATTCAAGAAGACAGGAAAGGTATCTAGGATGTACAATATTGATGCAGT
TCAAGCTATGGCCTTTGATGGTTGTTACCATGACAGCGAGATTGACATGGACCAGACTTCCCTCTCTC
TAAACTCAGAAGACTTCATGAGAAGAAAATCGATATGCAGCCCCAGCACCGATTCATCTCGGATAAAG
AGAAGAAAATCCCTAGGAGGACATGTTGGTAGAATCATTCTGGAAAACAACCATGTCATTGACACCTA
TTCTAGGATTTTATTCCCCATTGTGTATATTTTATTTAATTTGTTTTACTGGGGTGTATATGTATGAA
GGGGAATTTCAAATGT

| | | | |
|---|---|---|---|
| NOV38c, CG54683-02 | SEQ ID NO: 468 | 468 aa | MW at 54283.9kD |
| Protein Sequence | | | |

MVLAFQLVSFTYIWIILKPNVCAASNIKMTHQRCSSSMKQTCKQETRMKKDDSTKARPQKYEQLLHIE
DNDFAMRPGFGGSPVPVGIDAHVESIDSISETNMDFTMTFYLRHYWKDERLSFPSTANKSMTFDHRLT
RKIWVPDIFFVHSKRSFIHDTTMENIMLRVHPDGNVLLSLRITVSAMCFMDFSRFPLDDTQNCSLELE
SCAYNEDDLMLYWKHGNKSLNTEEHMSLSQFFIEDFSASSGLAFYSSTGWYNRLFINFVLRRHVFFFV
LQTYFPAILMVMLSWVSFWIDRRAVPARVSLGITTVLTMSTIITAVSASMPQVSYLKAVDVYLWVSSL
FVFLSVIEYAAVNYLTTVEERKQFKKTGKVSRMYNIDAVQAMAFDGCYHDSEIDMDQTSLSLNSEDFM
RRKSICSPSTDSSRIKRRKSLGGHVGRIILENNHVIDTYSRILFPIVYILFNLFYWGVYV

| | | |
|---|---|---|
| NOV38d, CG54683-03 | SEQ ID NO: 469 | 1438 bp |
| DNA Sequence | ORF Start: ATG at 21 | ORF Stop: TGA at 1419 |

GTTTTTTTGTTTTGGAAGAGATGGTCCTGGCTTTCCAGTTAGTCTCCTTCACCTACATCTGGATCATA
TTGAAACCAAATGTTTGTGCTGCTTCTAACATCAAGATGACACACCAGCGGTGCTCCTCTTCAATGAA
ACAAACCTGCAAACAAGAAACTAGAATGAAGAAAGATGACAGTACCAAAGCGCGGCCTCAGAAATATG
AGCAACTTCTCCATATAGAGGACAACGATTTCGCAATGAGACCTGGATTTGGAGGGTCTCCAGTGCCA
GTAGGTATAGATGTCCATGTTGAAAGCATTGACAGCATTTCAGAGACTAACATGGACTTTACAATGAC
TTTTTATCTCAGGCATTACTGGAAAGACGAGAGGCTCTCCTTTCCTAGCACAGCAAACAAAAGCATGA
CATTTGATCATAGATTGACCAGAAAGATCTGGGTGCCTGATATCTTTTTTGTCCACTCTAAAAGATCC
TTCATCCATGATACAACTATGGAGAATATCATGCTGCGCGTACACCCTGATGGAAACGTCCTCCTAAG
TCTCAGGATAACGGTTTCGGCCATGTGCTTTATGGATTTCAGCAGGTTTCCTCTGACTCAAAATTGTT
CTCTTGAACTGGAAAGCTGTGCCTACAATGAGGATGACCTAATGCTATACTGGAAACACGGAAACAAG
TCCTTAAATACTGAAGAACATATGTCCCTTTCTCAGTTCTTCATTGAAGACTTCAGTGCATCTAGTGG
ATTAGCTTTCTATAGCAGCACAGGTTGGTACAATAGGCTTTTCATCAACTTTGTGCTAAGGAGGCATG
TTTTCTTCTTTGTGCTGCAAACCTATTTCCCAGCCATATTGATGGTGATGCTTTCATGGGTTTCATTT
TGGATTGACCGAAGAGCTGTTCCTGCAAGAGTTTCCCTGGGTATCACCACAGTGCTGACCATGTCCAC
AATCATCACTGCTGTGAGCGCCTCCATGCCCCAGGTGTCCTACCTCAAGGCTGTGGATGTGTACCTGT
GGGTCAGCTCCCTCTTTGTGTTCCTGTCAGTCATTGAGTATGCAGCTGTGAACTACCTCACCACAGTG
GAAGAGCGGAAACAATTCAAGAAGACAGGAAAGGTATCTAGGATGTACAATATTGATGCAGTTCAAGC
TATGGCCTTTGATGGTTGTTACCATGACAGCGAGATTGACATGGACCAGACTTCCCTCTCTCTAAACT
CAGAAGACTTCATGAGAAGAAAATCGATATGCAGCCCCAGCACCGATTCATCTCGGATAAAGAGAAGA
AAATCCCTAGGAGGACATGTTGGTAGAATCATTCTGGAAAACAACCATGTCATTGACACCTATTCTAG
GATTTTATTCCCCATTGTGTATATTTTATTTAATTTGTTTTACTGGGGTGTATATGTATGAAGGGGAA
TTTCAAATGT

| | | | |
|---|---|---|---|
| NOV38d, GG54683-03 | SEQ ID NO: 470 | 466 aa | MW at 54081.8kD |
| Protein Sequence | | | |

MVLAFQLVSFTYIWIILKPNVCAASNIKMTHQRCSSSMKQTCKQETRMKKDDSTKARPQKYEQLLHIE
DNDFAMRPGFGGSPVPVGIDVHVESIDSISETNMDFTMTFYLRHYWKDERLSFPSTANKSMTFDHRLT
RKIWVPDIFFVHSKRSFIHDTThENIMLRVHPDGNVLLSLRITVSAMCFMDFSRFPLTQNCSLELESC
AYNEDDLMLYWKHGNKSLNTEEHMSLSQFFIEDFSASSGLAFYSSTGWYNRLFINFVLRRHVFFFVLQ
TYFPAILMVMLSWVSFWIDRRAVPARVSLGITTVLTMSTIITAVSASMPQVSYLKAVDVYLWVSSLFV
FLSVIEYAAVNYLTTVEERKQFKKTGKVSRMYNIDAVQAMAFDGCYHDSEIDMDQTSLSLNSEDFMRR
KSICSPSTDSSRIKRRKSLGGHVGRIILENNHVIDTYSRILFPIVYILFNLFYWGVYV

| | | |
|---|---|---|
| NOV38e, CG54683-04 | SEQ ID NO: 471 | 1799 bp |
| DNA Sequence | ORF Start: ATG at 71 | ORF Stop: TGA at 1445 |

AAGAAGAAACTGTGATCACAGTATTGGTTGCGTTCACCTGCATCCTTTCTGTTTTTTTGTTTTGGAAG
AGATGGTCCTGGCTTTCCAGTTAGTCTCCTTCACCTACATCTGGATCATATTGGTTTGTGCTGCTTCT
AACATCAAGATGACACACCAGCGGTGCTCCTCTTCAATGAAACAAACCGTAAGATGCTCAATGAAGAA
AGATGACAGTACCAAAGCGCGGCCTCAGAAATATGAGCAACTTCTCCATATAGAGGACAACGATITCG
CAATGAGACCTGGATTTGGAGGTTCTCCAGTGCCAGTAGGTATAGATGTCCATGTTGAAAGCATTGAC
AGCATTTCAGAGACTAACATGGACTTTACAATGACTTTTTATCTCAGGCATTACTGGAAAGACGAGAG
GCTCTCCTTTCCTAGCACAGCAAACAAAAGCATGACATTTGATCATAGAAAGAGTATCCCCCGCCCTG
AACACTTGCGGTATTCGTTATTCATCAGAAGGCTGTATCTGTTATACTGCCAGAGGTCTTTCTTCTCA
CCCTCATCCATACTTCCCTCATCTCCAGACATCCATGCACCTGGTACATCTAAAAGCAGTTTGTCTGA
TAGCCTTGTATGTATATCTGAAAAAAACTTGCCAGGACACAGTAAAAACACACCTCTTGCAATGGCCT
ACAATGAGGATGACCTAATGCTATACTGGAAACACGGAAACAAGTCCTTAAATACTGAAGAACATATG
TCCCTTTCTCAGTTCTTCATTGAAGACTTCAGTGCATCTAGTGGATTAGCTTTCTATAGCAGCACAGG
TACAGCATTTTACATGGGTGATTCATCAGCATTTATTGGACATCTACTGTTTTTAAATAGACATTTAC
ATTTCTTCATCATAAATTTTGAAATTACTCAAATATTGATGATTGGAATCACCACAGTGCTGACCATG
TCCACAATCATCACTGCTGTGAGCGCCTCCATGCCCCAGGTGTCCTACCTCAAGGCTGTGGATGTGTA
CCTGTGGGTCAGCTCCCTCTTTGTGTTCCTGTCAGTCATTGAGTATGCAGCTGTGAACTACCTCACCA
CAGTGGAAGAGCGGAAACAATTCAAGAAGACAGGAAAGGTACAGATTTCTAGGATGTACAATATTGAT
GCAGTTCAAGCTATGGCCTTTGATGGTTGTTACCATGACAGCGAGATTGACATGGACCAGACTTCCCT
CTCTCTAAACTCAGAAGACTTCATGAGAAGAAAATCGATATGCAGCCCCAGCACCGATTCATCTCGGA
TAAAGAGAAGAAAATCCCTAGGAGGACATGTTGGTAGAATCATTCTGGAAAACAACCATGTCATTGAC
ACCTATTCTAGGATTTTATTCCCCATTGTGTATATCCCATTGTGTATATCTTTATTTAATTTGTTTTA
CTGGGGTGTATATGTATGAAGGGGAATTTCAAATGTATACAACTTTAAAGCCAGATGATGTTTAAAAA
CAAAACTCTTGAATATGAGTTGGAATTGAAGACTTCAGTGCATCTAGTGGATTAGCTTTCTATAGCAG
CACAGGTACAGCATTTTACATGGGTGATTCATCAGCATTTATTGGACATCTACTGTTTTACTTTTGGT
CTTTGATGATGGTGATGTACAGATGGGTTGGAATCACCACAGTGCTGACCATGTCCACAATCATCACT
GCTGTGAGCGCCTCCATGCCCCAGGTGTCCTACCTCAAGGCTGTGGATGTGTACCTGTGGGTCAGCTC
CCTCTTTGTGTTCCTGTCAGTCATTGAGTAT

| | | | |
|---|---|---|---|
| NOV38e, GG54683-04 | SEQ ID NO: 472 | 458 aa | MW at 52330.5kD |
| Protein Sequence | | | |

MVLAFQLVSFTYIWIILVCAASNIKMTHQRCSSSMKQTVRCSMKKDDSTKARPQKYEQLLHIEDNDFA
MRPGFGGSPVPVGIDVHVESIDSISETNMDFTMTFYLRHYWKDERLSFPSTANKSMTFDHRKSIPRPE
HLRYSLFIRRLYLLYCQRSFFSPSSILPSSPDIHAPGTSKSSLSDSLVCISEKNLPGHSKNTPLAMAY
NEDDLMLYWKHGNKSLNTEEHMSLSQFFIEDFSASSGLAFYSSTGTAFYMGDSSAFIGHLLFLNRHLH
FFIINFEITQILMIGITTVLTMSTIITAVSASMPQVSYLKAVDVYLWVSSLFVFLSVIEYAAVNYLTT
VEERKQFKKTGKVQISRMYNIDAVQAMAFDGCYHDSEIDMDQTSLSLNSEDFMRRKSICSPSTDSSRI
KRRKSLGGHVGRIILENNHVIDTYSRILFPIVYIPLCISLFNLFYWGVYV

A ClustalW comparison of the above protein sequences yields the following sequence alignment shown in Table 38B.

TABLE 38B
Comparison of the NOV38 protein sequences.

NOV38a MVLAFQLVSFTYIWIILKPNVCAASNIKMTHQRCSSSMKQT--QETRMKKDDSTKARPQK
NOV38b MVLAFQLVSFTYIWIILKPNVCAASNIKMTHQRCSSSMKQTWMQETRMKKDDSTKARPQK
NOV38c MVLAFQLVSFTYIWIILKPNVCAASNIKMTHQRCSSSMKQTCKQETRMKKDDSTKARPQK
NOV38d MVLAFQLVSFTYIWIILKPNVCAASNIKMTHQRCSSSMKQTCKQETRMKKDDSTKARPQK
NOV38e MVLAFQLVSFTYIWIIL---VCAASNIKMTHQRCSSSMKQTVRCS--MKKDDSTKARPQK
NOV38a YEQLLHIEDNDFANRPGFGGSPVPVGIDVHVESIDSISETNNVDFTMTFYLRHYWKDERL
NOV38b YEQLLHIEDNDFANRPGFGGSPVPVGIDVHVESIDSISETN-MDFTMTFYLRHYWKDERL
NOV38c YEQLLHIEDNDFANRPGFGGSPVPVGIDAHVESIDSISETN-MDFTMTFYLRHYWKDERL
NOV38d YEQLLHIEDNDFANRPGFGGSPVPVGIDVHVESIDSISETN-MDFTMTFYLRHYWKDERL
NOV38e YEQLLHIEDNDFAMRPGFGGSPVPVGIDVHVESIDSISETN-MDFTMTFYLRHYWKDERL
NOV38a SFPSTANKSMTFDHR--------LTRKIWVPDIFFVHSKRSFIHDTTMENIMLRVHPDGN
NOV38b SFPSTANKSMTFDHR-------HLRYSLFIRRLYLLYCQRSFFSPSSILPSSPDIHAPG-
NOV38c SFPSTANKSMTFDHR--------LTRKIWVPDIFFVHSKRSFIHDTTMENIMLRVHPDGN
NOV38d SFPSTANKSMTFDHR--------LTRKIWVPDIFFVHSKRSFIHDTTMENIMLRVHPDGN
NOV38e SFPSTANKSMTFDHRKSIPRPEHLRYSLFIRRLYLLYCQRSFFSPSSILPSSPDIHAPG-
NOV38a VLLSLRITVSAMCFMDFSRFPLD-TQNCSLELES-AYNEDDLMLYWKHGNKSLNTEEHMS
NOV38b --TSKSSLSDSLVCISEKNLPG-HSKNTPLAMSDVAYNEDDLMLYWKHGNKSLNTEEHMS
NOV38c VLLSLRITVSANCFMDFSRFPLDDTQNCSLELESCAYNEDDLMLYWKHGNKSLNTEEHMS
NOV38d VLLSLRITVSANCFMDFSRFPL--TQNCSLELESCAYNEDDLMLYWKHGNKSLNTEEHMS
NOV38e --TSKSSLSDSLVCISEKNLPG-HSKNTPLAM---AYNEDDLMLYWKHGNKSLNTEEHNS
NOV38a LSQFFIEDFSASSGLAFYSSTGWYNRLFINFVLRRHVFFFVLQT-YFPAILMVMLSWVSF
NOV38b LSQFFIEDFSASSGLAFYSSTGTAFYMGDSSAFIGHLLFLIWSSRKRPGLEMLGLGILRI
NOV38c LSQFFIEDFSASSGLAFYSSTGWYNRLFINFVLRRHVFFFVLQT-YFPAILMVMLSWVSF
NOV38d LSQFFIEDFSASSGLAFYSSTGWYNRLFINFVLRRHVFFFVLQT-YFPAILMVMLSWVSF
NOV38e LSQFFIEDFSASSGLAFYSSTGTAFYMGDSSAFIGHLLFLN-----R---------HLHF
NOV38a WIDRRAVPARVSLGGITTVLTNSTIITAVSASMPQVSYLKAVDVYLWVSSLFVFLSVIEY
NOV38b WVITRAMDKKMEMG-ITTVLTMSTIITAVSASMPQVSYLKAVDVYLWVSSLFVFLSVIEY
NOV38c WIDRRAVPARVSLG-ITTVLTMSTIITAVSASMPQVSYLKAVDVYLWVSSLFVFLSVIEY
NOV38d WIDRRAVPARVSLG-ITTVLTMSTIITAVSASMPQVSYLKAVDVYLWVSSLFVFLSVIEY
NOV38e FIINFEITQILMIG-ITTVLTMSTIITAVSASMPQVSYLKAVDVYLWVSSLFVFLSVIEY
NOV38a AAVNYLTTVEERKQFKKT--GKISRMYNIDAVQAMAFDGCYHDSEIDMDQTSLSLNSEDF
NOV38b AAVNYLTTVEERKQFKKS-FSKISRMYNIDAVQAMAFDGCYHDSEIDMDQTSLSLNSEDF
NOV38c AAVNYLTTVEERKQFKKT--GKVSRNYNIDAVQAMAFDGCYHDSEIDMDQTSLSLNSEDF
NOV38d AAVNYLTTVEERKQFKKT--GKVSRMYNIDAVQAMAFDGCYHDSEIDMDQTSLSLNSEDF
NOV38e AAVNYLTTVEERKQFKKTGKVQISRNYNIDAVQAMAFDGCYHDSEIDMDQTSLSLNSEDF
NOV38a MRRKSICSPSTDSSRIKRRKSLGGHVGRIILENNHVIDTYSRIXFPIVYI-----LFNLF
NOV38b MRRKSICSPSTDSSRIKRRKSLGGHVGRIILENNHVIDTYSRILFPIVYI-----FFNLF
NOV38c MRRKSICSPSTDSSRIKRRKSLGGHVGRIILENNHVIDTYSRILFPIVYI-----LFNLF
NOV38d MRRKSICSPSTDSSRIKRRKSLGGHVGRIILENNHVIDTYSRILFPIVYI-----LFNLF
NOV38e MRRKSICSPSTDSSRIKRRKSLGGHVGRIILENNHVIDTYSRILFPIVYIPLCISLFNLF
NOV38a YWGVYV
NOV38b YWGVYV
NOV38c YWGVYV
NOV38d YWGVYV
NOV38e YWGVYV
NOV38a (SEQ ID NO: 464)
NOV38b (SEQ ID NO: 466)
NOV38c (SEQ ID NO: 468)
NOV38d (SEQ ID NO: 470)
NOV38e (SEQ ID NO: 472)

Further analysis of the NOV38a protein yielded the following properties shown in Table 38C.

TABLE 38C
Protein Sequence Properties NOV38a

| | |
|---|---|
| SignalP analysis: | Cleavage site between residues 25 and 26 |

PSORT II analysis:
PSG: a new signal peptide prediction method

N-region: length 0; pos.chg 0; neg.chg 0
H-region: length 17; peak value 9.53
PSG score: 5.12

GvH: von Heijne's method for signal seq. recognition

GvH score (threshold: −2.1) −7.09
possible cleavage site: between 24 and 25

>>> Seems to have no N-terminal signal peptide
ALOM: Klein et al's method for TM region allocation

| Init position for calculation: 1 | | |
|---|---|---|
| Tentative number of TMS(s) for the threshold 0.5: 6 | | |
| INTEGRAL | Likelihood = −4.51 | Transmembrane 1-17 |
| INTEGRAL | Likelihood = −0.43 | Transmembrane 171-187 |
| INTEGRAL | Likelihood = −3.98 | Transmembrane 265-281 |
| INTEGRAL | Likelihood = −0.64 | Transmembrane 300-316 |
| INTEGRAL | Likelihood = −3.98 | Transmembrane 329-345 |
| INTEGRAL | Likelihood = −0.85 | Transmembrane 448-464 |

| | |
|---|---|
| PERIPHERAL | Likelihood = 7.48 (at 229) |

ALOM score: −4.51 (number of TMSs: 6)

MTOP: Prediction of membrane topology (Hartmann et al.)

Center position for calculation: 8
Charge difference: 1.5 C(2.5) − N(1.0)
C > N: C-terminal side will be inside

>>>Caution: Inconsistent mtop result with signal peptide
>>> membrane topology: type 3b
MITDISC: discrimination of mitochondrial targeting seq

| | | | |
|---|---|---|---|
| R content: | 1 | Hyd Moment(75): | 2.39 |
| Hyd Moment(95): | 3.90 | G content: | 0 |
| D/E content: | 1 | S/T content: | 8 |

Score: −2.44

Gavel: prediction of cleavage sites for mitochondrial preseq

R-2 motif at 43 QRC|SS

NUCDISC: discrimination of nuclear localization signals

pat4: KRRK (5) at 422
pat7: none
bipartite: RRKSICSPSTDSSRIKR at 407
bipartite: RKSICSPSTDSSRIKRR at 408
content of basic residues: 10.3%
NLS Score: 0.99

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

55.6%: endoplasmic reticulum
11.1%: vacuolar
11.1%: Golgi
11.1%: vesicles of secretory system
11.1%: mitochondrial

>> prediction for CG54683-05 is end (k = 9)

A search of the NOV38a protein against the Geneseq database, a proprietary database that contains sequences published in patents and patent publication, yielded several homologous proteins shown in Table 38D.

TABLE 38D
Geneseq Results for NOV38a

| | | NOV38a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE21956 | Human transporter protein - Homo | 1 . . . 466 | 463/469 (98%) | 0.0 |
| | sapiens, 467 aa. [US2002028773- | 1 . . . 467 | 463/469 (98%) | |
| | A1, 07-MAR-2002] | | | |
| AAU04467 | Human gamma-amino butyric | 1 . . . 466 | 463/469 (98%) | 0.0 |
| | acid (GABA) receptor protein #1 - | 1 . . . 467 | 463/469 (98%) | |
| | Homo sapiens, 467 aa. | | | |
| | [WO200153489-A1, 26-JUL- | | | |
| | 2001] | | | |
| ABU12089 | Novel human gamma | 1 . . . 466 | 461/470 (98%) | 0.0 |
| | aminobutyric acid receptor-like | 1 . . . 468 | 462/470 (98%) | |
| | protein #2 - Homo sapiens, 468 | | | |
| | aa. [US2002123612-A1, 05-SEP- | | | |
| | 2002] | | | |
| AAG68256 | Human POLY3 protein sequence | 1 . . . 466 | 461/470 (98%) | 0.0 |
| | SEQ ID NO: 6 - Homo sapiens, | 1 . . . 468 | 462/470 (98%) | |
| | 468 aa. [WO200179294-A2, 25- | | | |
| | OCT-2001] | | | |
| ABU12090 | Novel human gamma | 1 . . . 466 | 461/469 (98%) | 0.0 |
| | aminobutyric acid receptor-like | 1 . . . 466 | 462/469 (98%) | |
| | protein #2 - Homo sapiens, 466 | | | |
| | aa. [US2002123612-A1, 05-SEP- | | | |
| | 2002] | | | |

In a BLAST search of public sequence databases, the NOV38a protein was found to have homology to the proteins shown in the BLASTP data in Table 38E.

TABLE 38E
Public BLASTP Results for NOV38a

| | | NOV38a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P50573 | Gamma-aminobutyric-acid | 1 . . . 466 | 392/470 (83%) | 0.0 |
| | receptor rho-3 subunit precursor | 1 . . . 464 | 417/470 (88%) | |
| | (GABA(A) receptor) - Rattus | | | |
| | norvegicus (Rat), 464 aa. | | | |
| Q9YGQ2 | Gamma-aminobutyric-acid | 1 . . . 465 | 300/478 (62%) | e−161 |
| | receptor rho-3 subunit - Morone | 4 . . . 469 | 369/478 (76%) | |
| | americana (White perch), 470 aa. | | | |
| P50572 | Gamma-aminobutyric-acid | 39 . . . 465 | 278/428 (64%) | e−155 |
| | receptor rho-1 subunit precursor | 50 . . . 473 | 326/428 (75%) | |
| | (GABA(A) receptor) - Rattus | | | |
| | norvegicus (Rat), 474 aa. | | | |
| P56475 | Gamma-aminobutyric-acid | 39 . . . 465 | 278/428 (64%) | e−154 |
| | receptor rho-1 subunit precursor | 50 . . . 473 | 326/428 (75%) | |
| | (GABA(A) receptor) - Mus | | | |
| | musculus (Mouse), 474 aa. | | | |
| P24046 | Gamma-aminobutyric-acid | 39 . . . 465 | 276/428 (64%) | e−154 |
| | receptor rho-1 subunit precursor | 49 . . . 472 | 325/428 (75%) | |
| | (GABA(A) receptor) - Homo | | | |
| | sapiens (Human), 473 aa. | | | |

PFam analysis indicates that the NOV38a protein contains the domains shown in the Table 38F.

TABLE 38F
Domain Analysis of NOV38a

| | NOV38a | Identities/ | |
|---|---|---|---|
| | Match | Similarities | Expect |
| Pfam Domain | Region | for the Matched Region | Value |
| Neur_chan_LBD | 58 . . . 265 | 74/253 (29%) | 1.6e−65 |
| | | 176/253 (70%) | |
| Neur_chan_memb | 272 . . . 462 | 45/292 (15%) | 9e−37 |
| | | 145/292 (50%) | |

### Example 39

The NOV39 clone was analyzed, and the nucleotide and encoded polypeptide sequences are shown in Table 39A.

TABLE 39A
NOV39 Sequence Analysis

| | | |
|---|---|---|
| NOV39a, CG54692-06 | SEQ ID NO: 473 | 1125 bp |
| DNA Sequence | ORF Start: ATG at 1 | ORF Stop: TGA at 1111 |

ATGGAGGCCGCTAGCCTTTCAGTGGCCACCGCCGGCGTTGCCCTTGCCCTGGGACCCGAGACCAGCAG
CGGGACCCCAAGCCCGAGAGGGATACTCGGTTCGACCCCGAGCGGCGCCGTCCTGCCGGGCCGAGGGC
CGCCCTTCTCTGTCTTCACGGTCCTGGTGGTGACGCTGCTAGTGCTGCTGATCGCTGCCACTTTCCTG
TGGAACCTGCTGGTTCCGGTCACCATCCCGCGGGTCCGTGCCTTCCACCGCGTGCCGCATAACTTGGT
GGCCTCGACGGCCGTCTCGGACGAACTAGTGGCAGCGCTGGCGATGCCACCGAGCCTGGCGAGTGAGC
TGTCGACCGGGCGACGTCGGCTGCTGGGCCGGAGCCTGTGCCACGTGTGGATCTCCTTCGACGCCCTG
TGCTGCCCCGCCGGCCTCGGGAACGTGGCGGCCATCGCCCTGGGCCGCGACGGGGCCATCACACGGCA
CCTGCAGCACACGCTGCGCACCCGCAGCCGCGCCTCGTTGCTCATGATCGCGCTCGCCCGGGTGCCGT
CGGCGCTCATCGCCCTCGCGCCGCTGCTCTTTGGCCGGGGCGAGGTGTGCGACGCTCGGCTCCAGCGC
TGCCAGGTGAGCCGGGAACCCTCCTATGCCGCCTTCTCCACCCGCGGCGCCTTCCACCTGCCGCTTGG
CGTGGTGCCGTTTGTCTACCGGAAGATCTACGAGGCGGCCAAGTTTCGTTTCGGCCGCCGCCGGAGAG
CTGTGCTGCCGTTGCCGGCCACCATGCAGGTGAAGGAAGCACCTGATGAGGCTGAAGTGGTGTTCACG
GCACATTGCAAAGCAACGGTGTCCTTCCAGGTGAGCGGGGACTCCTGGCGGGAGCAGAAGGAGAGGCG
AGCAGCCATGATGGTGGGAATTCTGATTGGCGTGTTTGTGCTGTGCTGGATCCCCTTCTTCCTGACGG
AACTCATCAGCCCACTCTGTGCCTGCAGCCTGCCCCCCATCTGGAAAAGCATATTTCTGTGGCTTGGC
TACTCCAATTCTTTCTTCAACCCCCTGATTTACACAGCTTTTAACAAGAACTACAACAATGCCTTCAA
GAGCCTCTTTACTAAGCAGAGATGAACACAGGGGTTA

| | | | |
|---|---|---|---|
| NOV39a, CG54692-06 | SEQ ID NO: 474 | 370 aa | MW at 40243.7kD |
| Protein Sequence | | | |

MEAASLSVATAGVALALGPETSSGTPSPRGILGSTPSGAVLPGRGPPFSVFTVLVVTLLVLLIAATFL
WNLLVPVTIPRVRAFHRVPHNLVASTAVSDELVAALAMPPSLASELSTGRRRLLGRSLCHVWISFDAL
CCPAGLGNVAAIALGRDGAITRHLQHTLRTRSRASLLMIALARVPSALIALAPLLFGRGEVCDARLQR
CQVSREPSYAAFSTRGAFHLPLGVVPFVYRKIYEAAKFRFGRRRRAVLPLPATMQVKEAPDEAEVVFT
AHCKATVSFQVSGDSWREQKERRAANMVGILIGVFVLCWIPFFLTELISPLCACSLPPIWKSIFLWLG
YSNSFFNPLIYTAFNKNYNNAFKSLFTKQR

| | | |
|---|---|---|
| NOV39b, CG54692-01 | SEQ ID NO: 475 | 1150 bp |
| DNA Sequence | ORF Start: ATG at 24 | ORF Stop: TGA at 1134 |

CTGGAGCTGCGATCCCAAGCGCCATGGAGGCCGCTAGCCTTTCAGTGGCCACCGCCGGCGTTGCCCTT
GCCCTGGGACCCGAGACCAGCAGCGGGACCCCAAGCCCGAGAGGGATACTCGGTTCGACCCCGAGCGG
CGCCGTCCTGCCGGGCCGAGGGCCGCCCTTCTCTGTCTTCACGGTCCTGGTGGTGACGCTGCTAGTGC
TGCTGATCGCTGCCACTTTCCTGTGGAACCTGCTGGTTCCGGTCACCATCCCGCGGGTCCGTGCCTTC
CACCGCGTGCCGCATAACTTGGTGGCCTCGACGGCCGTCTCGGACGAACTAGTGGCAGCGCTGGCGAT
GCCACCGAGCCTGGCGAGTGAGCTGTCGACCGGGCGACGTCGGCTGCTGGGCCGGAGCCTGTGCCACG
TGTGGATCTCCTTCGACGCCCTGTGCTGCCCCGCCGGCCTCGGGAACGTGGCGGCCATCGCCCTGGGC
CGCGACGGGGCCATCACACGGCACCTGCAGCACACGCTGCGCACCCGCAGCCGCGCCTCGTTGCTCAT
GATCGCGCTCGCCCGGGTGCCGTCGGCGCTCATCGCCCTCGCGCCGCTGCTCTTTGGCCGGGGCGAGG
TGTGCGACGCTCGGCTCCAGCGCTGCCAGGTGAGCCGGGAACCCTCCTATGCCGCCTTCTCCACCCGC
GGCGCCTTCCACCTGCCGCTTGGCGTGGTGCCGTTTGTCTACCGGAAGATCTACGAGGCGGCCAAGTT
TCGTTTCGGCCGCCGCCGGAGAGCTGTGCTGCCGTTGCCGGCCACCTCCAAGGTAAAGGAAGCACCTG
ATGAGGCTGAAGTGGTGTTCACGGCACATTGCAAAGCAACGGTGTCCTTCCAGGTGAGCGGGGACTCC
TGGCGGGAGCAGAAGGAGAGGCGAGCAGCCATGATGGTGGGAATTCTGATTGGCGTGTTTGTGCTGTG
CTGGATCCCCTTCTTCCTGACGGAACTCATCAGCCCACTCTGTGCCTGCAGCCTGCCCCCCATCTGGA
AAAGCATATTTCTGTGGCTTGGCTACTCCAATTCTTTCTTCAACCCCCTGATTTACACAGCTTTTAAC
AAGAACTACAACAATGCCTTCAAGAGCCTCTTTACTAAGCAGAGATGAACACAGGGGTTAGA

| | | | |
|---|---|---|---|
| NOV39b, CG54692-01 | SEQ ID NO: 476 | 370 aa | MW at 40199.7kD |
| Protein Sequence | | | |

MEAASLSVATAGVALALGPETSSGTPSPRGILGSTPSGAVLPGRGPPFSVFTVLVVTLLVLLIAATFL
WNLLVPVTIPRVRAFHRVPHNLVASTAVSDELVAALAMPPSLASELSTGRRRLLGRSLCHVWISFDAL
CCPAGLGNVAAIALGRDGAITRHLQHTLRTRSRASLLMIALARVPSALIALAPLLFGRGEVCDARLQR
CQVSREPSYAAFSTRGAFHLPLGVVPFVYRKIYEAAKFRFGRRRRAVLPLPATMQVKEAPDEAEVVFT
AHCKATVSFQVSGDSWREQKERRAANMVGILIGVFVLCWIPFFLTELISPLCACSLPPIWKSIFLWLG
YSNSFFNPLIYTAFNKNYNNAFKSLFTKQR

| | | |
|---|---|---|
| NOV39c, CG54692-02 | SEQ ID NO: 477 | 1150 bp |
| DNA Sequence | ORF Start: ATG at 24 | ORF Stop: TGA at 1134 |

CTGGAGCTGCGATCCCAAGCGCCATGGAGGCCGCTAGCCTTTCAGTGGCCACCGCCGGCGTTGCCCTT
GCCCTGGGACCCGAGACCAGCAGCGGGACCCCAAGCCCGAGAGGGATACTCGGTTCGACCCCGAGCGG
CGCCGTCCTGCCGGGCCGAGGGCCGCCCTTCTCTGTCTTCACGGTCCTGGTGGTGACGCTGCTAGTGC
TGCTGATCGCTGCCACTTTCCTGTGGAACCTGCTGGTTCCGGTCACCATCCCGCGGGTCCGTGCCTTC
CACCGCGTGCCGCATAACTTGGTGGCCTCGACGGCCGTCTCGGACGAACTAGTGGCAGCGCTGGCGAT
GCCACCGAGCCTGGCGAGTGAGCTGTCGACCGGGCGACGTCGGCTGCTGGGCCGGAGCCTGTGCCACG
TGTGGATCTCCTTCGACGCCCTGTGCTGCCCCGCCGGCCTCGGGAACGTGGCGGCCATCGCCCTGGGC
CGCGACGGGGCCATCACACGGCACCTGCAGCACACGCTGCGCACCCGCAGCCGCGCCTCGTTGCTCAT
GATCGCGCTCGCCCGGGTGCCGTCGGCGCTCATCGCCCTCGCGCCGCTGCTCTTTGGCCGGGGCGAGG
TGTGCGACGCTCGGCTCCAGCGCTGCCAGGTGAGCCGGGAACCCTCCTATGCCGCCTTCTCCACCCGC
GGCGCCTTCCACCTGCCGCTTGGCGTGGTGCCGTTTGTCTACCGGAAGATCTACGAGGCGGCCAAGTT
TCGTTTCGGCCGCCGCCGGAGAGCTGTGCTGCCGTTGCCGGCCACCTCCAAGGTAAAGGAAGCACCTG
ATGAGGCTGAAGTGGTGTTCACGGCACATTGCAAAGCAACGGTGTCCTTCCAGGTGAGCGGGGACTCC
TGGCGGGAGCAGAAGGAGAGGCGAGCAGCCATGATGGTGGGAATTCTGATTGGCGTGTTTGTGCTGTG
CTGGATCCCCTTCTTCCTGACGGAACTCATCAGCCCACTCTGTGCCTGCAGCCTGCCCCCCATCTGGA
AAAGCATATTTCTGTGGCTTGGCTACTCCAATTCTTTCTTCAACCCCCTGATTTACACAGCTTTTAAC
AAGAACTACAACAATGCCTTCAAGAGCCTCTTTACTAAGCAGAGATGAACACAGGGGTTAGA

| | | | |
|---|---|---|---|
| NOV39c, CG54692-02 | SEQ ID NO: 478 | 370 aa | MW at 40215.7kD |
| Protein Sequence | | | |

MEAASLSVATAGVALALGPETSSGTPSPRGILGSTPSGAVLPGRGPPFSVFTVLVVTLLVLLIAATFL
WNLLVPVTIPRVRAFHRVPHNLVASTAVSDELVAALAMPPSLASELSTGRRRLLGRSLCHVWISFDAL
CCPAGLGNVAAIALGRDGAITRHLQHTLRTRSRASLLMIALARVPSALIALAPLLFGRGEVCDARLQR
CQVSREPSYAAFSTRGAFHLPLGVVPFVYRKIYEAAKFRFGRRRRAVLPLPATMQVKEAPDEAEVVFT
AHCKATVSFQVSGDSWREQKERRAANMVGILIGVFVLCWIPFFLTELISPLCACSLPPIWKSIFLWLG
YSNSFFNPLIYTAFNKNYNNAFKSLFTKQR

| | | |
|---|---|---|
| NOV39d, GG54692-03 | SEQ ID NO: 479 | 1127 bp |
| DNA Sequence | ORF Start: ATG at 1 | ORF Stop: TGA at 1117 |

ATGGAGGCCGCTAGCCTTTCAGTGGCCACCGCCGGCGTTGCCCTTGCCCTGGGACCCGAGACCAGCAG
CGGGACCCCAAGCCCGAGAGGGATACTCGGTTCGACCCCGAGCGGCGCCGTCCTGCCGGGCCGAGGGC
CGCCCTTCTCTGTCTTCACGGTCCTGGTGGTGACGCTGCTAGTGCTGCTGATCGCCGCCACTTTCCTG
TGGAACCTGCTGGTTCCGGTCACCATCCCGCGGGTCCGTGCCTTCCACCGCGTGCCGCATAACTTGGT
GGCCTCGACGGCCGTCTCGGACGAACTAGTGGCAGCGCTGGCGATGCCACCGAGCCTGGCGAGTGAGC
TGTCGACCGGGCGACGTCGGCTGCTGGGCCGGAGCCTGTGCCACGTGTGGATCTCCTTCCACGGCCCA
CGGCTGTGCTGCCCCGCCGGCCTCGGGAACGTGGCGGCCATCGCCCTGGGCCGCGACGGGGCCATCAC
ACGGCACCTGCAGCACACGCTGCGCACCCGCAGCCGCGCCTCGTTGCTCATGATCGCGCTCACCCGGG
TGCCGTCGGCGCTCATCGCCCTCGCGCCGCTGCTCTTTGGCCGGGGCGAGGTGTGCGACGCTCGGCTC
CAGCGCTGCCAGGTGAGCCGGGAACCCTCCTATGCCGCCTTCTCCACCCGCGGCGCCTTCCACCTGCC
GCTTGGCGTGGTGCCGTTTGTCTACCGGAAGATCTACGAGGCGGCCAAGTTTCGTTTCGGCCGCCGCC
GGAGAGCTGTGCTGCCGTTGCCGGCCACCATGCAGGTGAAGGAAGCACCTGATGAGGCTGAAGTGGTG
TTCACGGCACATTGCAAAGCAACGGTGTCCTTCCAGGTGAGCGGGGACTCCTGGCGGGAGCAGAAGGA
GAGGCGAGCAGCCATGATGGTGGGAATTCTGATTGGCGTGTTTGTGCTGTGCTGGATCCCCTTCTTCC
TGACGGAACTCATCAGCCCACTCTGTGCCTGCAGCCTGCCCCCCATCTGGAAAAGCATATTTCTGTGG
CTTGGCTACTCCAATTCTTTCTTCAACCCCCTGATTTACACAGCTTTTAACAAGAACTACAACAATGC
CTTCAAGAGCCTCTTTACTAAGCAGAGATGAACACAGGG

| | | | |
|---|---|---|---|
| NOV39d, CG54692-03 | SEQ ID NO: 480 | 372 aa | MW at 40535.1kD |
| Protein Sequence | | | |

MEAASLSVATAGVALALGPETSSGTPSPRGILGSTPSGAVLPGRGPPFSVFTVLVVTLLVLLIAATFL
WNLLVPVTIPRVRAFHRVPHNLVASTAVSDELVAALAMPPSLASELSTGRRRLLGRSLCHVWISFHGP
RLCCPAGLGNVAAIALGRDGAITRHLQHTLRTRSRASLLMIALTRVPSALIALAPLLFGRGEVCDARL
QRCQVSREPSYAAFSTRGAFHLPLGVVPFVYRKIYEAAKFRFGRRRRAVLPLPATMQVKEAPDEAEVV
FTAHCKATVSFQVSGDSWREQKERRAAMMVGILIGVFVLCWIPFFLTELISPLCACSLPPIWKSIFLW
LGYSNSFFNPLIYTAFNKNYNNAFKSLFTKQR

| | | |
|---|---|---|
| NOV39e, GG54692-04 | SEQ ID NO: 481 | 1155 bp |
| DNA Sequence | ORF Start: ATG at 5 | ORF Stop: TGA at 1145 |

CGCCATGGAGGCCGCTAGCCTTTCAGTGGCCACCGCCGGCGTTGCCCTTGCCCCCGAGACCAGCAGCC
CGGCGTTGCCCTTGCCCTGGGACCCGAGACCAGCAGCAGGACCCGGGACCCCAAGCCCGAGAGGGATA
CTCGGTTCGACCCCGAGCGGCGCCGTCCTGCCGGGCCGAGGGCCGCCCTTCTCTGTCTTCACGGTCCT
GGTGGTGACGCTGCTAGTGCTGCTGATCGCTGCCACTTTCCTGTGGAACCTGCTGGTTCCGGTCACCA
TCCCGCGGGTCCGTGCCTTCCACCGCGTGCCGCATAACTTGGTGGCCTCGACGGCCGTCTCGGACGAA
CTAGTGGCAGCGCTGGCGATGCCACCGAGCCTGGCGAGTGAGCTGTCGACCGGGCGACGTCGGCTGCT
GGGCCGCCACGTGTGGATCTCCTTCGACGCCCTGTGCTGCCCCGCCGGCCTCGGGAACGTGGCGGCCA
TCGCCCTGGGCCGCGACGGGGCCATCACACGGCACCTGCAGCACACGCTGCGCACCCGCAGCCGCGCC
TCGTTGCTCATGATCGCGCTCGCCCGGGTGCCGTCGGCGCTCATCGCCCTCGCGCCGCTGCTCTTTGG
CCGGGGCGAGGTGTGCGACGCTCGGCTCCAGCGCTGCCAGGTGAGCCGGGAACCCTCCTATGCCGCCT
TCTCCACCCGCGGCGCCTTCCACCTGCCGCTTGGCGTGGTGCCGTTTGTCTACCGGAAGATCTACGAG
GCGGCCAAGTTTCGTTTCGGCCGCCGCCGGAGAGCTGTGCTGCCGTTGCCGGCCACCATGCAGGTGAA
GGAAGCACCTGATGAGGCTGAAGTGGTGTTCACGGCACATTGCAAAGCAACGGTGTCCTTCCAGGTGA
GCGGGGACTCCTGGCGGGAGCAGAAGGAGAGGCGAGCAGCCATGATGGTGGGAATTCTGATTGGCGTG
TTTGTGCTGTGCTGGATCCCCTTCTTCCTGACGGAACTCATCAGCCCACTCTGTGCCTGCAGCCTGCC
CCCCATCTGGAAAAGCATATTTCTGTGGCTTGGCTACTCCAATTCTTTCTTCAACCCCCTGATTTACA
CAGCTTTTAACAAGAACTACAACAATGCCTTCAAGAGCCTCTTTACTAAGCAGAGATGAACACAGGG

| | | | |
|---|---|---|---|
| NOV39e, CG54692-04 | SEQ ID NO: 482 | 380 aa | MW at 41306.9kD |
| Protein Sequence | | | |

MEAASLSVATAGVALAPETSSPALPLPWDPRPAAGPGTPSPRGILGSTPSGAVLPGRGPPFSVFTVLV
VTLLVLLIAATFLWNLLVPVTIPRVRAFHRVPHNLVASTAVSDELVAALANPPSLASELSTGRRRLLG
RHVWISFDALCCPAGLGNVAAIALGRDGAITRHLQHTLRTRSRASLLMIALARVPSALIALAPLLFGR
GEVCDARLQRCQVSREPSYAAFSTRGAFHLPLGVVPFVYRKIYEAAKFRFGRRRRAVLPLPATMQVKE
APDEAEVVFTAHCKATVSFQVSGDSWREQKERRAANMVGILIGVFVLCWIPFFLTELISPLCACSLPP
IWKSIFLWLGYSNSFFNPLIYTAFNKNYNNAFKSLFTKQR

| | | |
|---|---|---|
| NOV39f, GG54692-05 | SEQ ID NO: 483 | 1152 bp |
| DNA Sequence | ORF Start: ATG at 5 | ORF Stop: TGA at 1142 |

CGCCATGGAGGCCGCTAGCCTTTCAGTGGCCACCGCCGGCGTTGCCCTTGCCCTGGGACCCGAGACCA
GCAGCGGACCCGGGACCCCAAGCCCGAGAGGGATACTCGGTTCGACCCCGAGCGGCGCCGTCCTGCCG
GGCCGAGGGCCGCCCTTCTCTGTCTTCACGGTCCTGGTGGTGACGCTGCTAGTGCTGCTGATCGCTGC
CACTTTCCTGTGGAACCTGCTGGTTCCGGTCACCATCCCGCGGGTCCGTGCCTTCCACCGCGTGCCGC
ATAACTTGGTGGCCTCGACGGCCGTCTCGGACGAACTAGTGGCAGCGCTGGCGATGCCACCGAGCCTG
GCGAGTGAGCTGTCGACCGGGCGACGTCGGCTGCTGGGCCGGAGCCTGTGCCACGTGTGGATCTCCTT
CGACGCCGGAGCCTGTCTGTGCTGCCCCGCCGGCCTCGGGAACGTGGCGGCCATCGCCCTGGGCCGCG
ACGGGGCCATCACACGGCACCTGCAGCACACGCTGCGCACCCGCAGCCGCGCCTCGTTGCTCATGATC
GCGCTCGCCCGGGTGCCGTCGGCGCTCATCGCCCTCGCGCCGCTGCTCTTTGGCCGGGGCGAGGTGTG
CGACGCTCGGCTCCAGCGCTGCCAGGTGAGCCGGGAACCCTCCTATGCCGCCTTCTCCACCCGCGGCG
CCTTCCACCTGCCGCTTGGCGTGGTGCCGTTTGTCTACCGGAAGATCTACGAGGCGGCCAAGTTTCGT
TTCGGCCGCCGCCGGAGAGCTGTGCTGCCGTTGCCGGCCACCATGCAGGTGAGGTCCAAGGTAAAGGA
AGCACCTGATGAGGCTGAAGTGGTGTTCACGGCACATTGCAAAGCAACGGTGTCCTTCCAGGTGAGCG
GGGACTCCTGGCGGGAGCAGAAGGAGAGGCGAGCAGCCATGATGGTGGGAATTCTGATTGGCGTGTTT
GTGCTGTGCTGGATCCCCTTCTTCCTGACGGAACTCATCAGCCCACTCTGTGCCTGCAGCCTGCCCCC
CATCTGGAAAAGCATATTTCTGTGGCTTGGCTACTCCAATTCTTTCTTCAACCCCCTGATTTACACAG
CTTTTAACAAGAACTACAACAATGCCTTCAAGAGCCTCTTTACTAAGCAGAGATGAACACAGGG

| | | | |
|---|---|---|---|
| NOV39f, CG54692-05 | SEQ ID NO: 484 | 379 aa | MW at 41099.7kD |
| Protein Sequence | | | |

MEAASLSVATAGVALALGPETSSGPGTPSPRGILGSTPSGAVLPGRGPPFSVFTVLVVTLLVLLIAAT
FLWNLLVPVTIPRVRAFHRVPHNLVASTAVSDELVAALAMPPSLASELSTGRRRLLGRSLCHVWISFD
AGACLCCPAGLGNVAAIALGRDGAITRHLQHTLRTRSRASLLMIALARVPSALIALAPLLFGRGEVCD
ARLQRCQVSREPSYAAFSTRGAFHLPLGVVPFVYRKIYEAAKFRFGRRRRAVLPLPATMQVRSKVKEA
PDEAEVVFTAHCKATVSFOVSGDSWREQKERRAAMMVGILIGVFVLCWIPFFLTELISPLCACSLPPI
WKSIFLWLGYSNSFFNPLIYTAFNKNYNNAFKSLFTKQR

A ClustalW comparison of the above protein sequences yields the following sequence alignment shown in Table 39B.

TABLE 39B
Comparison of the NOV39 protein sequences.

NOV39a MEAASLSVATAGVALA-----------LGPETSSG--TPSPRGILGSTPSGAVLPGRGPP
NOV39b MEAASLSVATAGVALA-----------LGPETSSG--TPSPRGILGSTPSGAVLPGRGPP
NOV39c MEAASLSVATAGVALA-----------LGPETSSG--TPSPRGILGSTPSGAVLPGRGPP
NOV39d MEAASLSVATAGVALA-----------LGPETSSG--TPSPRGILGSTPSGAVLPGRGPP
NOV39e MEAASLSVATAGVAIAPETSSPALPLPWDPRPAAGPGTPSPRGILGSTPSGAVLPGRGPP
NOV39f MEAASLSVATAGVALA-----------LGPETSSGPGTPSPRGILGSTPSGAVLPGRGPP
NOV39a FSVFTVLVVTLLVLLIAATFLWNLLVPVTIPRVRAFHRVPHNLVASTAVSDELVAALAMP
NOV39b FSVFTVLVVTLLVLLIAATFLWNLLVPVTIPRVRAFHRVPHNLVASTAVSDELVAALAMP
NOV39c FSVFTVLVVTLLVLLIAATFLWNLLVPVTIPRVRAFHRVPHNLVASTAVSDELVAALAMP
NOV39d FSVFTVLVVTLLVLLIAATFLWNLLVPVTIPRVRAFHRVPHNLVASTAVSDELVAALAMP
NOV39e FSVFTVLVVTLLVLLIAATFLWNLLVPVTIPRVRAFHRVPHNLVASTAVSDELVAALANP
NOV39f FSVFTVLVVTLLVLLIAATFLWNLLVPVTIPRVRAFHRVPHNLVASTAVSDELVAALAMP
NOV39a PSLASELSTGRRRLLGRSLCHVWISFDA---LCCPAGLGNVAAIALGRDGAITRHLQHTL
NOV39b PSLASELSTGRRRLLGRSLCHVWISFDA---LCCPAGLGNVAAIALGRDGAITRHLQHTL
NOV39c PSLASELSTGRRRLLGRSLCHVWISFDA---LCCPAGLGNVAAIALGRDGAITRHLQHTL
NOV39d PSLASELSTGRRRLLGRSLCHVWISFHG-PRLCCPAGLGNVAAIALGRDGAITRHLQHTL
NOV39e PSLASELSTGRRRLLGR---HVWISFDA---LCCPAGLGNVAAIALGRDGAITRHLQHTL
NOV39f PSLASELSTGRRRLLGRSLCHVWISFDAGACLCCPAGLGNVAAIALGRDGAITRHLQHTL
NOV39a RTRSRASLLMIALARVPSALIALAPLLFGRGEVCDARLQRCQVSREPSYAAFSTRGAFHL
NOV39b RTRSRASLLMIALARVPSALIALAPLLFGRGEVCDARLQRCQVSREPSYAAFSTRGAFHL
NOV39c RTRSRASLLMIALARVPSALIALAPLLFGRGEVCDARLQRCQVSREPSYAAFSTRGAFHL
NOV39d RTRSRASLLMIALTRVPSALIALAPLLFGRGEVCDARLQRCQVSREPSYAAFSTRGAFHL
NOV39e RTRSRASLLMIALARVPSALIALAPLLFGRGEVCDARLQRCQVSREPSYAAFSTRGAFHL
NOV39f RTRSRASLLMIALARVPSALIALAPLLFGRGEVCDARLQRCQVSREPSYAAFSTRGAFHL
NOV39a PLGVVPFVYRKIYEAAKFRFGRRRRAVLPLPAT----MQVKEAPDEAEVVFTAHCKATVS
NOV39b PLGVVPFVYRKIYEAAKFRFGRRRRAVLPLPAT----SKVKEAPDEAEVVFTAHCKATVS
NOV39c PLGVAPFVYRKIYEAAKFRFGRRRRAVLPLPAT----MQVKEAPDEAEVVFTAHCKATVS
NOV39d PLGVVPFVYRKIYEAAKFRFGRRRRAVLPLPAT----MQVKEAPDEAEVVFTAHCKATVS
NOV39e PLGVVPFVYRKIYEAAKFRFGRRRRAVLPLPAT----MQVKEAPDEAEVVFTAHCKATVS
NOV39f PLGVVPFVYRKIYEAAKFRFGRRRRAVLPLPATMQVRSKVKEAPDEAEVVFTAHCKATVS
NOV39a FQVSGDSWREQKERRAANMVGILIGVFVLCWIPFFLTELISPLCACSLPPIWKSIFLWLG
NOV39b FQVSGDSWREQKERRAAMMVGILIGVFVLCWIPFFLTELISPLCACSLPPIWKSIFLWLG
NOV39c FQVSGDSWREQKERRAAMMVGILIGVFVLCWIPFFLTELISPLCACSLPPIWKSIFLWLG
NOV39d FQVSGDSWREQKERRAAMMVGILIGVFVLCWIPFFLTELISPLCACSLPPIWKSIFLWLG
NOV39e FQVSGDSWREQKERRAANMVGILIGVFVLCWIPFFLTELISPLCACSLPPIWKSIFLWLG
NOV39f FQVSGDSWREQKERRAAMMVGILIGVFVLCWIPFFLTELISPLCACSLPPIWKSIFLWLG
NOV39a YSNSFFNPLIYTAFNKNYNNAFKSLFTKQR
NOV39b YSNSFFNPLIYTAFNKNYNNAFKSLFTKQR
NOV39c YSNSFFNPLIYTAFNKNYNNAFKSLFTKQR
NOV39d YSNSFFNPLIYTAFNKNYNNAFKSLFTKQR
NOV39e YSNSFFNPLIYTAFNKNYNNAFKSLFTKQR
NOV39f YSNSFFNPLIYTAFNKNYNNAFKSLFTKQR
NOV39a (SEQ ID NO: 474)
NOV39b (SEQ ID NO: 476)
NOV39c (SEQ ID NO: 478)
NOV39d (SEQ ID NO: 480)
NOV39e (SEQ ID NO: 482)
NOV39f (SEQ ID NO: 484)

Further analysis of the NOV39a protein yielded the following properties shown in Table 39C.

TABLE 39C
Protein Sequence Properties NOV39a

| | |
|---|---|
| SignalP analysis: | Cleavage site between residues 25 and 26 |

PSORT II analysis:
PSG: a new signal peptide prediction method

N-region: length 2; pos.chg 0; neg.chg 1
H-region: length 17; peak value 0.00
PSG score: −4.40

GvH: von Heijne's method for signal seq. recognition

GvH score (threshold: −2.1): −4.24
possible cleavage site: between 23 and 24

>>> Seems to have no N-terminal signal peptide
ALOM: Klein et al's method for TM region allocation

| Init position for calculation: 1 | | |
|---|---|---|
| Tentative number of TMS(s) for the threshold 0.5: 5 | | |
| INTEGRAL | Likelihood = 0.32 | Transmembrane 1-17 |
| INTEGRAL | Likelihood = −12.10 | Transmembrane 48-64 |
| INTEGRAL | Likelihood = −0.48 | Transmembrane 135-151 |
| INTEGRAL | Likelihood = −4.94 | Transmembrane 172-188 |
| INTEGRAL | Likelihood = −9.66 | Transmembrane 300-316 |

| | |
|---|---|
| PERIPHERAL | Likelihood = 0.69 (at 90) |

ALOM score: −12.10 (number of TMSs: 5)

MTOP: Prediction of membrane topology (Hartmann et al.)

Center position for calculation: 8
Charge difference: 0.0 C(0.0) − N(0.0)
N >= C: N-terminal side will be inside

>>> membrane topology: type 3a
MITDISC: discrimination of mitochondrial targeting seq

| | | | |
|---|---|---|---|
| R content: | 0 | Hyd Moment(75): | 3.56 |
| Hyd Moment(95): | 5.35 | G content: | 2 |
| D/E content: | 2 | S/T content: | 3 |

Score: −7.53

Gavel: prediction of cleavage sites for mitochondrial preseq

R-2 motif at 95 HRV|PH

NUCDISC: discrimination of nuclear localization signals

pat4: RRRR (5) at 246
pat7: none
bipartite: RKIYEAAKFRFGRRRRA at 234
content of basic residues: 10.5%
NLS Score: 0.50

KDEL: ER retention motif in the C-terminus: none
ER Membrane Retention Signals:

KKXX-like motif in the C-terminus: FTKQ

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

66.7%: endoplasmic reticulum
22.2%: mitochondrial
11.1%: nuclear

>> prediction for CG54692-06 is end (k = 9)

A search of the NOV39a protein against the Geneseq database, a proprietary database that contains sequences published in patents and patent publication, yielded several homologous proteins shown in Table 39D.

TABLE 39D
Geneseq Results for NOV39a

| | | NOV39a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE18654 | Human G-protein coupled | 1 . . . 370 | 370/370 (100%) | 0.0 |
| | receptor (GCREC-15) - Homo | 1 . . . 370 | 370/370 (100%) | |
| | sapiens, 370 aa. [WO200210387- | | | |
| | A2, 07-FEB-2002] | | | |
| AAM47212 | Human NOV5b protein - Homo | 1 . . . 370 | 369/370 (99%) | 0.0 |
| | sapiens, 370 aa. [WO200174851- | 1 . . . 370 | 369/370 (99%) | |
| | A2, 11-OCT-2001] | | | |
| AAM47211 | Human NOV5a protein - Homo | 1 . . . 370 | 368/370 (99%) | 0.0 |
| | sapiens, 370 aa. [WO200174851- | 1 . . . 370 | 369/370 (99%) | |
| | A2, 11-OCT-2001] | | | |
| AAE15638 | Human G-protein coupled | 1 . . . 370 | 367/372 (98%) | 0.0 |
| | receptor-8 (GCREC-8) protein - | 1 . . . 372 | 367/372 (98%) | |
| | Homo sapiens, 372 aa. | | | |
| | [WO200198351-A2, 27-DEC- | | | |
| | 2001] | | | |
| ABB78809 | Human NOV5 protein sequence | 1 . . . 370 | 370/379 (97%) | 0.0 |
| | SEQ ID NO: 16 - Homo sapiens, | 1 . . . 379 | 370/379 (97%) | |
| | 379 aa. [WO200230974-A2, 18- | | | |
| | APR-2002] | | | |

In a BLAST search of public sequence databases, the NOV39a protein was found to have homology to the proteins shown in the BLASTP data in Table 39E.

TABLE 39E
Public BLASTP Results for NOV39a

| | | NOV39a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD13088 | Sequence 11 from Patent | 1 . . . 370 | 369/370 (99%) | 0.0 |
| | WO0174851 - Homo sapiens | 1 . . . 370 | 369/370 (99%) | |
| | (Human), 370 aa. | | | |
| CAD13087 | Sequence 9 from Patent | 1 . . . 370 | 368/370 (99%) | 0.0 |
| | WO0174851 - Homo sapiens | 1 . . . 370 | 369/370 (99%) | |
| | (Human), 370 aa. | | | |
| P35365 | 5-hydroxytryptamine 5B receptor | 1 . . . 370 | 296/370 (80%) | e−165 |
| | (5-HT-5B) (Serotonin receptor) | 1 . . . 370 | 316/370 (85%) | |
| | (MR22) - Rattus norvegicus | | | |
| | (Rat), 370 aa. | | | |
| P31387 | 5-hydroxytryptamine 5B receptor | 1 . . . 370 | 298/370 (80%) | e−165 |
| | (5-HT-5B) (Serotonin receptor) - | 1 . . . 370 | 317/370 (85%) | |
| | Mus musculus (Mouse), 370 aa. | | | |
| S38744 | serotonin receptor 5B - rat, 369 | 1 . . . 370 | 296/370 (80%) | e−164 |
| | aa. | 1 . . . 369 | 316/370 (85%) | |

PFam analysis indicates that the NOV39a protein contains the domains shown in the Table 39F.

TABLE 39F
Domain Analysis of NOV39a

| | NOV39a | Identities/ | |
|---|---|---|---|
| | Match | Similarities | |
| Pfam Domain | Region | for the Matched Region | Expect Value |
| 7tm_1 | 69 . . . 351 | 75/314 (24%) | 1.2e−43 |
| | | 197/314 (63%) | |

### Example 40

The NOV40 clone was analyzed, and the nucleotide and encoded polypeptide sequences are shown in Table 40A.

TABLE 40A
NOV40 Sequence Analysis

| | | |
|---|---|---|
| NOV40a, CG55069-01 | SEQ ID NO: 485 | 8657 bp |
| DNA Sequence | ORF Start: ATG at 151 | ORF Stop: TAA at 8326 |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGTATCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40a, CG55069-01 | SEQ ID NO: 486 | 2725 aa | MW at 303959.6kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIANHLFGLNWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCINGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNQPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQANGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNNTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGALALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40b, CG55069-04 | SEQ ID NO: 487 | 1783 bp |
| DNA Sequence | ORF Start: at 7 | ORF Stop: at 778 |

AAGCTTTGTCCCCGAAATTGCCATGGAAATGGAGAATGCGTTTCTGGAACTTGCCATTGTTTTCCAGG
ATTTCTGGGTCCGGATTGTTCAAGAGCCGCCTGTCCAGTGTTATGTAGTGGCAACGGGCAGTACTCCA
AGGGCCGCTGCCTGTGTTTCAGCGGCTGGAAGGGCACCGAGTGTGATGTGCCGACTACCCAGTGTATT
GACCCACAGTGTGGGGGTCGTGGGATTTGTATCATGGGCTCCTGTGCTTGCAACTCAGGATACAAAGG
AGAAAGTTGTGAAGAAGCTGACTGTATAGACCCTGGGTGTTCTAATCATGGTGTGTGTATCCACGGGG
AATGTCACTGCAGTCCAGGATGGGGAGGTAGCAATTGTGAAATACTGAAGACCATGTGTCCAGACCAG
TGCTCCGGCCACGGAACGTATCTTCAAGAAAGTGGCTCCTGCACGTGTGACCCTAACTGGACTGGCCC
AGACTGCTCAAACGAAATATGTTCTGTGGACTGTGGCTCACACGGCGTTTGCATGGGGGGGACGTGTC
GCTGTGAAGAAGGCTGGACGGGCCCAGCCTGTAATCAGAGAGCCTGCCACCCCCGCTGTGCCGAGCAC
GGGACCTGCAAGGATGGCAAGTGTGAATGCAGCCAGGGCTGGAATGGAGAGCACTGCACTATCGAGGG
TTGTCCTGGTCTGTGCAACAGCAATGGAAGATGTACCCTGGACCAAAATGGCTGGCATTGTGTGTGCC
AGCCTGGATGGAGAGGAGCAGGCTGTGACGTCGAC

| | | | |
|---|---|---|---|
| NOV40b, CG55069-04 | SEQ ID NO: 488 | 257 aa | MW at 26866.7kD |
| Protein Sequence | | | |

CPRNCHGNGECVSGTCHCFPGFLGPDCSRAACPVLCSGNGQYSKGRCLCFSGWKGTECDVPTTQCIDP
QCGGRGICIMGSCACNSGYKGESCEEADCIDPGCSNHGVCIHGECHCSPGWGGSNCEILKTMCPDQCS
GHGTYLQESGSCTCDPNWTGPDCSNEICSVDCGSHGVCMGGTCRCEEGWTGPACNQRACHPRCAEHGT
CKDGKCECSQGWNGEHCTIEGCPGLCNSNGRCTLDQNGWHCVCQPGWRGAGCD

| | | |
|---|---|---|
| NOV40c, 248993047 | SEQ ID NO: 489 | 2448 bp |
| DNA Sequence | ORF Start: at 1 | ORF Stop: end of sequence |

GGTACCAACTGGCAGCTACAGCAGACTGAAAATGACACATTTGAGAATGGAAAAGTGAATTCTGATAC
CATGCCAACAAACACTGTGTCATTACCTTCTGGAGACAATGGAAAATTAGGTGGATTTACGCAAGAAA
ATAACACCATAGATTCCGGAGAACTTGATATTGGCCGAAGAGCAATTCAAGAGATTCCTCCCGGGATC
TTCTGGAGATCACAGCTCTTCATTGATCAGCCACAGTTTCTTAAATTCAATATCTCTCTTCAGAAGGA
TGCATTGATTGGAGTATATGGCCGGAAAGGCTTACCGCCTTCCCATACTCAGTATGACTTCGTGGAGC
TCCTGGATGGCAGCAGGCTGATTGCCAGAGAGCAGCGGAGCCTGCTTGAGACGGAGAGAGCCGGGCGG
CAGGCGAGATCCGTCAGCCTTCATGAGGCCGGCTTTATCCAGTACTTGGATTCTGGAATCTGGCATCT
GGCTTTTTATAATGATGGGAAAAATGCAGAGCAGGTGTCTTTTAATACCATTGTTATAGAGTCTGTGG
TGGAATGTCCCCGAAATTGCCATGGAAATGGAGAATGCGTTTCTGGAACTTGCCATTGTTTTCCAGGA
TTTCTGGGTCCGGATTGTTCAAGAGCCGCCTGTCCAGTGTTATGTAGTGGCAACGGGCAGTACTCCAA
GGGCCGCTGCCTGTGTTTCAGCGGCTGGAAGGGCACCGAGTGTGATGTGCCGACTACCCAGTGTATTG
ACCCACAGTGTGGGGGTCGTGGGATTTGTATCATGGGCTCTTGTGCTTGCAACTCAGGATACAAAGGA
GAAAGTTGTGAAGAAGCTGACTGTATAGACCCTGGGTGTTCTAATCATGGTGTGTGTATCCACGGGGA
ATGTCACTGCAGTCCAGGATGGGGAGGTAGCAATTGTGAAATACTGAAGACCATGTGTCCAGACCAGT
GCTCCGGCCACGGAACGTATCTTCAAGAAAGTGGCTCCTGCACGTGTGACCCTAACTGGACTGGCCCA
GACTGCTCAAACGAAATATGTTCTGTGGACTGTGGCTCACACGGCGTTTGCATGGGGGGGACGTGTCG
CTGTGAAGAAGGCTGGACGGGCCCAGCCTGTAATCAGAGAGCCTGCCACCCCCGCTGTGCCGAGCACG
GGACCTGCAAGGATGGCAAGTGTGAATGCAGCCAGGGCTGGAATGGAGAGCACTGCACTATCGAGGGT
TGTCCTGGTCTGTCCAACAGCAATGGAAGATGTACCCTGGACCAAAATGGCTGGCATTGTGTGTGCCA
GCCTGGATGGAGAGGAGCAGGCTGTGACGTAGCCATGGAGACTCTTTGCACAGATAGCAAGGACAATG
AAGGAGATGGACTCATTGACTGCATGGATCCCGATTGCTGCCTACAGAGTTCCTGCCAGAATCAGCCC
TATTGTCGGGGACTGCCGGATCCTCAGGACATCATTAGCCAAAGCCTTCAATCGCCTTCTCAGCAAGC
TGCCAAATCCTTTTATGATCGAATCAGTTTCCTTATAGGATCTGATAGCACCCATGTTATACCTGGAG
AAAGTCCTTTCAATAAGAGCCTTGCATCTGTCATCAGAGGCCAAGTACTGACTGCTGATGGAACTCCA
CTTATTGGAGTAAATGTCTCGTTTTTCCATTACCCAGAATATGGATATACTATTACCCGCCAGGACGG
AATGTTTGACTTGGTGGCAAATGGTGGGGCCTCTCTAACTTTGGTATTTGAACGATCCCCATTCCTCA
CTCAGTATCATACTGTGTGGATTCCATGGAATGTCTTTTATGTGATGGATACCCTAGTCATGAAGAAA
GAAGAGAATGACATTCCCAGCTGTGATCTGAGTGGATTCGTGAGGCCAAATCCCATCATTGTGTCATC
ACCTTTATCCACCTTTTTCAGATCTTCTCCTGAAGACAGTCCCATCATTCCCGAAACACAGGTACTCC
ACGAGGAAACTACAATTCCAGGAACAGATTTGAAACTCTCCTACTTGAGTTCCAGAGCTGCAGGGTAT
AAGTCAGTTCTCAAGATCACCATGACCCAGTCTATTATTCCATTTAATTTAATGAAGGTTCATCTTAT
GGTAGCTGTAGTAGGAAGACTCTTCCAAAAGTGGTTTCCTGCCTCACCAAACTTGGCCTATACTTTCA
TATGGGATAAAACAGATGCATATAATCAGAAAGTCTATGGTCTATCTGAAGCTGTTGTGTCAGTTGGA
TATGAGTATGAGTCGTGTTTGGACCTGACTcTGTGGGAAAAGAGGACTGCCATTCTGCAGGGCTATGA
ATTGGATGCGTCCAACATGGGTGGCTGGACATTAGATAAACATCACGTGCTGGATGTACAGAACGGTA
TACTGTACAAGGGAAACGGGGAAAACCAGTTCATCTCCCAGCAGCCTCCAGTCGTGAGTAGCGTCGAC

| | | | |
|---|---|---|---|
| NOV40c, 248993047 | SEQ ID NO: 490 | 816 aa | MW at 89174.1kD |
| Protein Sequence | | | |

GTNWQLQQTENDTFENGKVNSDTMPTNTVSLPSGDNGKLGGFTQENNTIDSGELDIGRRAIQEIPPGI
FWRSQLFIDQPQFLKFNISLQKDALIGVYGRKGLPPSHTQYDFVELLDGSRLIAREQRSLLETERAGR
QARSVSLHEAGFIQYLDSGIWHLAFYNDGKNAEQVSFNTIVIESVVECPRNCHGNGECVSGTCHCFPG
FLGPDCSRAACPVLCSGNGQYSKGRCLCFSGWKGTECDVPTTQCIDPQCGGRGICIMGSCACNSGYKG
ESCEEADCIDPGCSNHGVCIHGECHCSPGWGGSNCEILKTMCPDQCSGHGTYLQESGSCTCDPNWTGP
DCSNEICSVDCGSHGVCMGGTCRCEEGWTGPACNQRACHPRCAEHGTCKDGKCECSQGWNGEHCTIEG
CPGLSNSNGRCTLDQNGWHCVCQPGWRGAGCDVAMETLCTDSKDNEGDGLIDCMDPDCCLQSSCQNQP
YCRGLPDPQDIISQSLQSPSQQAAKSFYDRISFLIGSDSTHVIPGESPFNKSLASVIRGQVLTADGTP
LIGVNVSFFHYPEYGYTITRQDGMFDLVANGGASLTLVFERSPFLTQYHTVWIPWNVFYVMDTLVMKK
EENDIPSCDLSGFVRPNPIIVSSPLSTFFRSSPEDSPIIPETQVLHEETTIPGTDLKLSYLSSRAAGY
KSVLKITMTQSIIPFNLMKVHLMVAVVGRLFQKWFPASPNLAYTFIWDKTDAYNQKVYGLSEAVVSVG
YEYESCLDLTLWEKRTAILQGYELDASNMGGWTLDKHHVLDVQNGILYKGNGENQFISQQPPVVSSVD

| | | |
|---|---|---|
| NOV40d, 262802488 | SEQ ID NO: 491 | 2519 bp |
| DNA Sequence | ORF Start: at 1 | ORF Stop: TAG at 2515 |

GGTACCAACTGGCAGCTACAGCAGACTGAAAATGACGCATTTGAGAATGGAAAAGTGAATTCTGATAC
CATGCCAACAAACACTGTGTCATTACCTTCTGGAGACAATGGAAAATTAGGTGGATTTACGCAAGAAA
ATAACACCATAGATTCCGGAGAACTTGATATTGGCCGAAGAGCAATTCAAGAGATTCCTCCCGGGATC
TTCTGGAGATCACAGCTCTTCATTGATCAGCCACAGTTTCTTAAATTCAATATCTCTCTTCAGAAGGA
TGCATTGATTGGAGTATATGGCCGGAAAGGCTTACCGCCTTCCCATACTCAGTATGACTTCGTGGAGC
TCCTGGATGGCAGCAGGCTGATTGCCAGAGAGCAGCGGAGCCTGCTTGAGACGGAGAGAGCCGGGCGG
CAGGCGAGATCCGTCAGCCTTCATGAGGCCGGCTTTATCCAGTACTTGGATTCTGGAATCTGGCATCT
GGCTTTTTATAATGATGGGAAAAATGCAGAGCAGGTGTCTTTTAATACCATTGTTATAGAGTCTGTGG
TGGAATGTCCCCGAAATTGCCATGGAAATGGAGAATGCGTTTCTGGAACTTGCCATTGTTTTCCAGGA
TTTCTGGGTCCGGATTGTTCAAGAGCCGCCTGTCCAGTGTTATGTAGTGGCAACGGGCAGTACTCCAA
GGGCCGCTGCCTGTGTTTCAGCGGCTGGAAGGGCACCGAGTGTGATGTGCCGACTACCCAGTGTATTG
ACCCACAGTGTGGGGGTCGTGGGATTTGTATCATGGGCTCCTGTGCTTGCAACTCAGGATACAAAGGA
GAAAGTTGTGAAGAAGCTGACTGTATAGACCCTGGGTGTTCTAATCATGGTGTGTGTATCCACGGGGA
ATGTCACTGCAGTCCAGGATGGGGAGGTAGCAATTGTGAAATACTGAAGACCATGTGTCCAGACCAGT
GCTCCGGCCACGGAACGTATCTTCAAGAAAGTGGCTCCTGCACGTGTGACCCTAACTGGACTGGCCCA
GACTGCTCAAACGAAATATGTTCTGTGGACTGTGGCTCACACGGCGTTTGCATGGGGGGGACGTGCCG
CTGTGAAGAAGGCTGGACGGGCCCAGCCTGTAATCAGAGAGCCTGCCACCCCCGCTGTGCCGAGCACG
GGACCTGCAAGGATGGCAAGTGTGAATGCAGCCAGGGCTGGAATGGAGAGCACTGCACTATCGCTCAC
TATTTGGATAAGATAGTTAAAGACAAGATAGGATATAAAGAGGGTTGTCCTGGTCTGTGCAACAGCAA
TGGAAGATGTACCCTGGACCAAAATGGCTGGCATTGTGCGTGCCAGCCTGGATGGAGAGGAGCAGGCT
GTGACGTAGCCATGGAGACTCTTTGCACAGATAGCAAGGACAATGAAGGAGATGGACTCATTGACTGC
ATGGATCCCGATTGCTGCCTACAGAGTTCCTGCCAGAATCAGCCCTATTGTCGGGGACTGCCGGATCC
TCAGGGCATCATTAGCCAAAGCCTTCAATCGCCTTCTCAGCAAGCTGCCAAATCCTTTTATGATCGAA
TCAGTTTCCTTATAGGATCTGATAGCACCCATGTTATACCTGGAGAAAGTCCTTTCAATAAGAGCCTT
GCATCTGTCATCAGAGGCCAAGTACTGACTGCTGATGGAACTCCACTTATTGGAGTAAATGTCTCGTT
TTTCCATTACCCAGAATATGGATATACTATTACCCGCCAGGACGGAATGTTTGACTTGGTGGCAAATG
GTGGGGCCTCTCTAACTTTGGTATTTGAACGATCCCCATTCCTCACTCAGTATCATACTGTGTGGATT
CCATGGAATGTCTTTTATGTGATGGATACCCTAGTCATGAAGAAAGAAGAGAATGACATTCCCAGCTG
TGATCTGAGTGGATTCGTGAGGCCAAATCCCATCATTGTGTCATCACCTTTATCCACCTTTTTCAGAT
CTTCTCCTGAAGACAGTCCCATCATTCCCGAAACACAGGTACTCCACGAGGAAACTACAATTCCAGGA
ACAGATTTGAAACTCTCCTACTTGAGTTCCAGAGCTGCAGGGTATAAGTCAGTTCTCAAGATCACCAT
GACCCAGTCTATTATTCCATTTAATTTAATGAAGGTTCATCTTATGGTAGCTGTAGTAGGAAGACTCT
TCCAAAAGTGGTTTCCTGCCTCACCAAACTTGGCCTATACTTTCATATGGGATAAAACAGATGCATAT
AATCAGAAAGTCTATGGTCTATCTGAAGCTGTTGTGTCAGTTGGATATGAGTATGAGTCGTGTTTGGA
CCTGACTCTGTGGGAAAAGAGGACTGCCATTCTGCAGGGCTATGAATTGGATGCGTCCAACATGGGTG
GCTGGACATTAGATAAACATCACGTGCTGGATGTACAGAACGGTATACTGTACAAGGGAAACGGGGAA
AACCAGTTCATCTCCCAGCAGCCTCCAGTCGTGAGTAGCGTCGACCCAGCAGCCTCCAGTCGTGAGTA
GCG

| | | | |
|---|---|---|---|
| NOV40d, 262802488 | SEQ ID NO: 492 | 838 aa | MW at 91545.9kD |
| Protein Sequence | | | |

GTNWQLQQTENDAFENGKVNSDTMPTNTVSLPSGDNGKLGGFTQENNTIDSGELDIGRRAIQEIPPGI
FWRSQLFIDQPQFLKFNISLQKDALIGVYGRKGLPPSNTQYDFVELLDGSRLIAREQRSLLETERAGR
QARSVSLHEAGFIQYLDSGIWHLAFYNDGKNAEQVSFNTIvIESVVECPRNCHGNGECVSGTCHCFPG
FLGPDCSRAACPVLCSGNGQYSKGRCLCFSGWKGTECDVPTTQCIDPQCGGRGICIMGSCACNSGYKG
ESCEEADCIDPGCSNHGVCINGECHCSPGWGGSNCEILKTMCPDQCSGHGTYLQESGSCTCDPNWTGP
DCSNEICSVDCGSHGVCMGGTCRCEEGWTGPACNQRACHPRCAEHGTCKDGKCECSQGWNGEHCTIAH
YLDKIVKDKIGYKEGCPGLCNSNGRCTLDQNGWHCACQPGWRGAGCDVAMETLCTDSKDNEGDGLIDC
MDPDCCLQSSCQNQPYCRGLPDPQGIISQSLQSPSQQAAKSFYDRISFLIGSDSTHVIPGESPFNKSL
ASVIRGQVLTADGTPLIGVNVSFFHYPEYGYTITRQDGMFDLVANGGASLTLVFERSPFLTQYHTVWI
PWNVFYVMDTLVMKKEENDIPSCDLSGFVRPNPIIVSSPLSTFFRSSPEDSPIIPETQVLHEETTIPG
TDLKLSYLSSRAAGYKSVLKITMTQSIIPFNLMKVHLMVAVVGRLFQKWFPASPNLAYTFIWDKTDAY
NQKVYGLSEAVVSVGYEYESCLDLTLWEKRTAILQGYELDASNNGGWTLDKMHVLDVQNGILYKGNGE
NQFISQQPPVVSSVDPAASSRE

| | | |
|---|---|---|
| NOV40e, 248993606 | SEQ ID NO: 493 | 2536 bp |
| DNA Sequence | ORF Start: at 1 | ORF Stop: at 2536 |

GGTACCAACTGGCAGCTACAGCAGACTGAAAATGACACATTTGAGAATGGAAAAGTGAATTCTGATAC
CATGCCAACAAACACTGTGTCATTACCTTCTGGAGACAATGGAAAATTAGGTGGATTTACGCAAGAAA
ATAACACCATAGATTCCGGAGAACTTGATATTGGCCGAAGAGCAATTCAAGAGATTCCTCCCGGGATC
TTCTGGAGATCACAGCTCTTCATTGATCAGCCACAGTTTCTTAAATTCAATATCTCTCTTCAGAAGGA
TGCATTGATTGGAGTATATGGCCGGAAAGGCTTACCGCCTTCCCATACTCAGTATGACTTCGTGGAGC
TCCTGGATGGCAGCAGGCTGATTGCCAGAGAGCAGCGGAGCCTGCTTGAGACGGAGAGAGCCGGGCGG
CAGGCGAGATCCGTCAGCCTTCATGAGGCCGGCTTTATCCAGTACTTGGATTCTGGAATCTGGCATCT
GGCTTTTTATAATGATGGGAAAAATGCAGAGCAGGTGTCTTTTAATACCATTGTTATAGAGTCTGTGG
TGGAATGTCCCCGAAATTGCCATGGAAATGGAGAATGCGTTTCTGGAACTTGCCATTGTTTTCCAGGA
TTTCTGGGTCCGGATTGTTCAAGAGCCGCCTGTCCAGTGTTATGTAGTGGCAACGGGCAGTACTCCAA
GGGCCGCTGCCTGTGTTTCAGCGGCTGGAAGGGCACCGAGTGTGATGTGCCGACTACCCAGTGTATTG
ACCCACAGTGTGGGGGTCGTGGGATTTGTATCATGGGCTCTTGTGCTTGCAACTCAGGATACAAAGGA
GAAAGTTGTGAAGAAGCTGACTGTATAGACCCTGGGTGTTCTAATCATGGTGTGTGTATCCACGGGGA
ATGTCACTGCAGTCCAGGATGGGGAGGTAGCAATTGTGAAATACTGAAGACCATGTGTCCAGACCAGT
GCTCCGGCCACGGAACGTATCTTCAAGAAAGTGGCTCCTGCACGTGTGACCCTAACTGGACTGGCCCA
GACTGCTCAAACGAAATATGTTCTGTGGACTGTGGCTCACACGGCGTTTGCATGGGGGGGACGTGTCG
CTGTGAAGAAGGCTGGACGGGCCCAGCCTGTAATCAGAGAGCCTGCCACCCCCGCTGTGCCGAGCACG
GGACCTGCAAGGATGGCAAGTGTGAATGCAGCCAGGGCTGGAATGGAGAGCACTGCACTATCGCTCAC
TATTTGGATAAGATAGTTAAAGAGGGTTGTCCTGGTCTGTGCAACAGCAATGGAAGATGTACCCTGGA
CCAAAATGGCTGGCATTGTGTGTGCCAGCCTGGATGGAGAGGAGCAGGCTGTGACGTAGCCATGGAGA
CTCTTTGCACAGATAGTAAGGACAATGAAGGAGATGGACTCATTGACTGCATGGATCCCGATTGCTGC
CTACAGAGTTCCTGCCAGAATCAGCCCTATTGTCGGGGACTGCCGGATCCTCAGGACATCATTAGCCA
AAGCCTTCAATCGCCTTCTCAGCAAGCTGCCAAATCCTTTTATGATCGAATCAGTTTCCTTATAGGAT
CTGATAGCACCCATGTTATACCTGGAGAAAGTCCTTTCAATAAGAGCCTTGCATCTGTCATCAGAGGC
CAAGTACTGACTGCTGATGGAACTCCACTTATTGGAGTAAATGTCTCGTTTTTCCATTACCCAGAATA
TGGATATACTATTACCCGCCAGGACGGAATGTTTGACTTGGTGGCAAATGGTGGGGCCTCTCTAACTT
TGGTATTTGAACGATCCCCATTCCTCACTCAGTATCATACTGTGTGGATTCCATGGAATGTCTTTTAT
GTGATGGATACCCTAGTCATGAAGAAAGAAGAGAATGACATTCCCAGCTGTGATCTGAGTGGATTCGT
GAGGCCAAATCCCATCATTGTGTCATCACCTTTATCCACCTTTTTCAGATCTTCTCCTGAAGACAGTC
CCATCATTCCCGAAACACAGGTACTCCACGAGGAAACTACAATTCCAGGAACAGATTTGAAACTCTCC
TACTTGAGTTCCAGAGCTGCAGGGTATAAGTCAGTTCTCAAGATCACCATGACCCAGTCTATTATTCC
ATTTAATTTAATGAAGGTTCATCTTATGGTAGCTGTAGTAGGAAGACTCTTCCAAAAGTGGTTTCCTG
CCTCACCAAACTTGGCCTATACTTTCATATGGGATAAAACAGATGCATATAATCAGAAAGTCTATGGT
CTATCTGAAGCTGTTGTGTCAGTTGGATATGAGTATGAGTCGTGTTTGGACCTGACTCTGTGGGAAAA
GAGGACTGCCATTCTGCAGGGCTATGAATTGGATGCGTCCAACATGGGTGGCTGGACATTAGATAAAC
ATCACGTGCTGGATGTACAGAACGGTATACTGTACAAGGGAAACGGGGAAAACCAGTTCATCTCCCAG
CAGCCTCCAGTCGTGAGTAGCGTCGACGCTACACACGACTGGAGGCTGCTGGTCATTTTCAGCTCTGC
AGTAGCTGCCAGTTGGTACC

| | | | |
|---|---|---|---|
| NOV40e, 248993606 | SEQ ID NO: 494 | 845 aa | MW at 92534.0kD |
| Protein Sequence | | | |

GTNWQLQQTENDTFENGKVNSDTNPTNTVSLPSGDNGKIGGFTQENNTIDSGELDIGRRAIQEIPPGI
FWRSQLFIDQPQFLKFNISLQKDALIGVYGRKGLPPSHTQYDFVELLDGSRLIAREQRSLLETERAGR
QARSVSLHEAGFIQYLDSGIWHLAFYNDGKNAEQVSFNTIVIESVVECPRNCHGNGECVSGTCHCFPG
FLGPDCSRAACPVLCSGNGQYSKGRCLCFSGWKGTECDVPTTQCIDPQCGGRGICIMGSCACNSGYKG
ESCEEADCIDPGCSNHGVCIHGECHCSPGWGGSNCEILKTMCPDQCSGHGTYLQESGSCTCDPNWTGP
DCSNEICSVDCGSHGVCMGGTCRCEEGWTGPACNQRACHPRCAEHGTCKDGKCECSQGWNGEHCTIAH
YLDKIVKEGCPGLCNSNGRCTLDQNGWHCVCQPGWRGAGCDVAMETLCTDSKDNEGDGLIDCMDPDCC
LQSSCQNQPYCRGLPDPQDIISQSLQSPSQQAAKSFYDRISFLIGSDSTHVIPGESPFNKSLASVIRG
QVLTADGTPLIGVNVSFFHYPEYGYTITRQDGNFDLVANGGASLTLVFERSPFLTQYHTVWIPWNVFY
VMDTLVMKKEENDIPSCDLSGFVRPNPIIVSSPLSTFFRSSPEDSPIIPETQVLHEETTIPGTDLKLS
YLSSRAAGYKSVLKITMTQSIIPFNLMKVHLMVAVVGRLFQKWFPASPNLAYTFIWDKTDAYNQKVYG
LSEAVVSVGYEYESCLDLTLWEKRTAILQGYELDASNMGGWTLDKHHVLDVQNGILYKGNGENQFISQ
QPPVVSSVDATHDWRLLVIFSSAVAASWY

| | | |
|---|---|---|
| NOV40f, 314411758 | SEQ ID NO: 495 | 2500 bp |
| DNA Sequence | ORF Start: at 2 | ORF Stop: end of sequence |

CACCAAGCTTAACTGGCAGCTACAGCAGACTGAAAATGACGCATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGGCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGGCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCATTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGGGCACCGAGTGTGATGTGCCGACTACCCAGTGT
ATTGACCCACAGTGTGGGGGTCGTGGGATTTGTATCATGGGCTCCTGTGCTTGCAACTCAGGATACAA
AGGAGAAAGTTGTGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGTGCTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GCCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGACAAGATAGGATATAAAGAGGGTTGTCCTGGTCTGTGCAACA
GCAATGGAAGATGTACCCTGGACCAAAATGGCTGGCATTGTGCGTGCCAGCCTGGATGGAGAGGAGCA
GGCTGTGACGTAGCCATGGAGACTCTTTGCACAGATAGCAAGGACAATGAAGGAGATGGACTCATTGA
CTGCATGGATCCCGATTGCTGCCTACAGAGTTCCTGCCAGAATCAGCCCTATTGTCGGGGACTGCCGG
ATCCTCAGGGCATCATTAGCCAAAGCCTTCAATCGCCTTCTCAGCAAGCTGCCAAATCCTTTTATGAT
CGAATCAGTTTCCTTATAGGATCTGATAGCACCCATGTTATACCTGGAGAAAGTCCTTTCAATAAGAG
CCTTGCATCTGTCATCAGAGGCCAAGTACTGACTGCTGATGGAACTCCACTTATTGGAGTAAATGTCT
CGTTTTTCCATTACCCAGAATATGGATATACTATTACCCGCCAGGACGGAATGTTTGACTTGGTGGCA
AATGGTGGGGCCTCTCTAACTTTGGTATTTGAACGATCCCCATTCCTCACTCAGTATCATACTGTGTG
GATTCCATGGAATGTCTTTTATGTGATGGATACCCTAGTCATGAAGAAAGAAGAGAATGACATTCCCA
GCTGTGATCTGAGTGGATTCGTGAGGCCAAATCCCATCATTGTGTCATCACCTTTATCCACCTTTTTC
AGATCTTCTCCTGAAGACAGTCCCATCATTCCCGAAACACAGGTACTCCACGAGGAAACTACAATTCC
AGGAACAGATTTGAAACTCTCCTACTTGAGTTCCAGAGCTGCAGGGTATAAGTCAGTTCTCAAGATCA
CCATGACCCAGTCTATTATTCCATTTAATTTAATGAAGGTTCATCTTATGGTAGCTGTAGTAGGAAGA
CTCTTCCAAAAGTGGTTTCCTGCCTCACCAAACTTGGCCTATACTTTCATATGGGATAAAACAGATGC
ATATAATCAGAAAGTCTATGGTCTATCTGAAGCTGTTGTGTCAGTTGGATATGAGTATGAGTCGTGTT
TGGACCTGACTCTGTGGGAAAAGAGGACTGCCATTCTGCAGGGCTATGAATTGGATGCGTCCAACATG
GGTGGCTGGACATTAGATAAACATCACGTGCTGGATGTACAGAACGGTATACTGTACAAGGGAAACGG
GGAAAACCAGTTCATCTCCCAGCAGCCTCCAGTCGTGAGTAGCCTCGAGGGC

| | | | |
|---|---|---|---|
| NOV40f, 314411758 | SEQ ID NO: 496 | 833 aa | MW at 91116.5kD |
| Protein Sequence | | | |

TKLNWQLQQTENDAFENGKVNSDTMPTNTVSLPSGDNGKLGGFTQENNTIDSGELDIGRRAIQEIPPG
IFWRSQLFIDQPQFLKFNISLQKDALIGVYGRKGLPPSHTQYDFVELLDGSRLIAREQRSLLETERAG
RQARSVSLHEAGFIQYLDSGIWHLAFYNDGKNAEQVSFNTIVIESVVECPRNCHGNGECVSGTCHCFP
GFLGPDCSRAACPVLCSGNGQYSKGRCLCFSGWKGTECDVPTTQCIDPQCGGRGICIMGSCACNSGYK
GESCEEADCIDPGCSNHGVCIHGECHCSPGWGGSNCEILKTMCPDQCSGHGTYLQESGSCTCDPNWTG
PDCSNEICSVDCGSHGVCMGGTCRCEEGWTGPACNQRACHPRCAEHGTCKDGKCECSQGWNGEHCTIA
HYLDKIVKDKIGYKEGCPGLCNSNGRCTLDQNGWHCACQPGWRGAGCDVANETLCTDSKDNEGDGLID
CMDPDCCLQSSCQNQPYCRGLPDPQGIISQSLQSPSQQAAKSFYDRISFLIGSDSTHVIPGESPFNKS
LASVIRGQVLTADGTPLIGVNVSFFHYPEYGYTITRQDGMFDLVANGGASLTLVFERSPFLTQYHTVW
IPWNVFYVMDTLVMKKEENDIPSCDLSGFVRPNPIIVSSPLSTFFRSSPEDSPIIPETQVLHEETTIP
GTDLKLSYLSSRAAGYKSVLKITMTQSIIPFNLMKVHLMVAVVGRLFQKWFPASPNLAYTFIWDKTDA
YNQKVYGLSEAVVSVGYEYESCLDLTLWEKRTAILQGYELDASNNGGWTLDKHHVLDVQNGILYKGNG
ENQFISQQPPVVSSLEG

| | | |
|---|---|---|
| NOV40g, 319067006 | SEQ ID NO: 497 | 730 bp |
| DNA Sequence | ORF Start: at 2 | ORF Stop: end of sequence |

CACCTCGCGAACAGAACCTTCTTACGAACTTGTGAAGAGTCAGCAGTGGGATGATATACCGCCCATCT
TCGGAGTCCAGCAGCAAGTGGCGCGGCAGGCCAAGGCCTTCCTGTCGCTGGGGAAGATGGCCGAGGTG
CAGGTGAGCCGGCGCCGGGCCGGCGGCGCGCAGTCCTGGCTGTGGTTCGCCACGGTCAAGTCGCTGAT
CGGCAAGGGCGTCATGCTGGCCGTCAGCCAGGGCCGCGTGCAGACCAACGTGCTCAACATCGCCAACG
AGGACTGCATCAAGGTGGCGGCCGTGCTCAACAACGCCTTCTACCTGGAGAACCTGCACTTCACCATC
GAGGGCAAGGACACGCACTACTTCATCAAGACCACCACGCCCGAGAGCGACCTGGGCACGCTGCGGTT
GACCAGCGGCCGCAAGGCGCTGGAGAACGGCATCAACGTGACGGTGTCGCAGTCCACCACGGTGGTGA
ACGGCAGGACGCGCAGGTTCGCGGACGTGGAGATGCAGTTCGGCGCGCTGGCGCTGCACGTGCGCTAC
GGCATGACCCTGGACGAGGAGAAGGCGCGCATCCTGGAGCAGGCGCAGCAGCGCGTGCGCGACGGCAA
GGTGCAGGGCTACGACGGGTACTACGTACTCTCGGTGGAGCAGTACCCCGAGCTGGCCGACAGCGCCA
ACAACATCCAGTTCCTGCGGCAGAGCGAGATCGGCAGGAGGGTCGACGGC

| | | | |
|---|---|---|---|
| NOV40g, 319067006 | SEQ ID NO: 498 | 243 aa | MW at 27175.5kD |
| Protein Sequence | | | |

TSRTEPSYELVKSQQWDDIPPIFGVQQQVARQAKAFLSLGKMAEVQVSRRRAGGAQSWLWFATVKSLI
GKGVMLAVSQGRVQTNVLNIANEDCIKVAAVLNNAFYLENLHFTIEGKDTHYFIKTTTPESDLGTLRL
TSGRKALENGINVTVSQSTTVVNGRTRRFADVEMQFGALALHVRYGMTLDEEKARILEQAQQRVRDGK
VQGYDGYYVLSVEQYPELADSANNIQFLRQSEIGRRVDG

| | | |
|---|---|---|
| NOV40h, 319506086 | SEQ ID NO: 499 | 1178 bp |
| DNA Sequence | ORF Start: at 78 | ORF Stop: end of sequence |

CACCTCGCGACCAATGAGACTTGAACCCTGAGCCTAAGTTGTCACCAGCAGGACTGATGTGCACACAG
AAGGAATGAAGTATGGATGTGAAAGAACGCAGGCCTTACTGCTCCCTGACCAAGAGCAGACGAGAGAA
GGAACGGCGCTACACAAATTCCTCCGCAGACAATGAGGAGTGCCGGGTACCCACACAGAAGTCCTACA
GTTCCAGCGAGACATTGAAAGCTTTTGATCATGATTCCTCGCGGCTGCTTTACGGCAACAGAGTGAAG
GATTTGGTTCACAGAGAAGCAGACGAGTTCACTAGACAAGAGCAACCTGCAAGCAATCAAGGCCAGTC
TACCCTGCAGCCCTTGCCGCCTTCCCATAAGCAGCACTCTGCACAGCATCATCCATCCATCACTTCTC
TCAACAGAAACTCCCTGACCAATAGAAGGAACCAGAGTCCGGCCCCGCCGGCTGCTTTGCCCGCCGAG
CTGCAAACCACACCCGAGTCCGTCCAGCTGCAGGACAGCTGGGTCCTTGGCAGTAATGTACCACTGGA
AAGCAGGCATTTCCTATTCAAAACAGGAACAGGTACAACGCCACTGTTCAGTACTGCAACCCCAGGAT
ACACAATGGCATCTGGCTCTGTTTATTCACCACCTACTCGGCCACTACCTAGAAACACCCTATCAAGA
AGTGCTTTTAAATTCAAGAAGTCTTCAAAGTACTGTAGCTGGAAATGCACTGCACTGTGTGCCGTAGG
GGTCTCGGTGCTCCTGGCAATACTCCTGTCTTATTTTATAGCAATGCATCTCTTTGOCCTCAACTGGC
AGCTACAGCAGACTGAAAATGACACATTTGAGAATGGAAAAGTGAATTCTGATACCATGCCAACAAAC
ACTGTGTCATTACCTTCTGGAGACAATGGAAAATTAGGTGGATTTACGCAAGAAAATAACACCATAGA
TTCCGGAGAACTTGATATTGGCCGAAGAGCAATTCAAGAGATTCCTCCCGGGATCTTCTGGAGATCAC
AGCTCTTCATTGATCAGCCACAGTTTCTTAAATTCAATATCTCTCTTCAGAAGGATGCATTGATTGGA
GTATATGGCCGGAAAGGCTTACCGCCTTCCCATACTCAGTATGACTTCGTGGAGCTCCTGGATGGCAG
CAGGCTGATTGCCCTCGAGGGC

| | | | |
|---|---|---|---|
| NOV40h, 319506086 | SEQ ID NO: 500 | 367 aa | MW at 40968.5kD |
| Protein Sequence | | | |

SMDVKERRPYCSLTKSRREKERRYTNSSADNEECRVPTQKSYSSSETLKAFDHDSSRLLYGNRVKDLV
HREADEFTRQEQPASNQGQSTLQPLPPSHKQHSAQHHPSITSLNRNSLTNRRNQSPAPPAALPAELQT
TPESVQLQDSWVLGSNVPLESRHFLFKTGTGTTPLFSTATPGYTMASGSVYSPPTRPLPRNTLSRSAF
KFKKSSKYCSWKCTALCAVGVSVLLAILLSYFIAMHLFGLNWQLQQTENDTFENGKVNSDTMPTNTVS
LPSGDNGKLGGFTQENNTIDSGELDIGRRAIQEIPPGIFWRSQLFIDQPQFLKFNISLQKDALIGVYG
RKGLPPSHTQYDFVELLDGSRLIALEG

| | | |
|---|---|---|
| NOV40i, CG55069-03 | SEQ ID NO: 501 | 8473 bp |
| DNA Sequence | ORF Start: ATG at 258 | ORF Stop: TAA at 8142 |

TTGACAGAAAAAGGCAGTAAACGGGGAATCTCTTTTTTTGAATAAAGAAGAAGAAGAAATAAAGTACC
TGTCATCTTGACAAGTGGCGGAGCGGAGGAGTCAAGGATTATAAATGATCACAGCCAGGTCCAGCTCG
CCCCGTGATTGGGCTCTCCCGCGATCTGCACCGGGGGAAGCGCATGAGAGGCCAATGAGACTTGAACC
CTGAGCCTAAGTTGTCACCAGCAGGACTGATGTGCACACAGAAGGAATGAAGTATGGATGTGAAAGAA
CGCAGGCCTTACTGCTCCCTGACCAAGAGCAGACGAGAGAAGGAACGGCGCTACACAAATTCCTCCGC
AGACAATGAGGAGTGCCGGGTACCCACACAGAAGTCCTACAGTTCCAGCGAGACATTGAAAGCTTTTG
ATCATGATTCCTCGCGGCTGCTTTACGGCAACAGAGTGAAGGATTTGGTTCACAGAGAAGCAGACGAG
TTCACTAGACAAGAGCAACCTGCAAGCAATCAAGGCCAGTCTACCCTGCAGCCCTTGCCGCCTTCCCA
TAAGCAGCACTCTGCACAGCATCATCCATCCATCACTTCTCTCAACAGAAACTCCCTGACCAATAGAA
GGAACCAGAGTCCGGCCCCGCCGGCTGCTTTGCCCGCCGAGCTGCAAACCACACCCGAGTCCGTCCAG
CTGCAGGACAGCTGGGTCCTTGGCAGTAATGTACCACTGGAAAGCAGGCATTTCCTATTCAAAACAGG
AACAGGTACAACGCCACTGTTCAGTACTGCAACCCCAGGATACACAATGGCATCTGGCTCTGTTTATT
CACCACCTACTCGGCCACTACCTAGAAACACCCTATCAAGAAGTGCTTTTAAATTCAAGAAGTCTTCA
AAGTACTGTAGCTGGAAATGCACTGCACTGTGTGCCGTAGGGGTCTCGGTGCTCCTGGCAATACTCCT
GTCTTATTTTATAGCAATGCATCTCTTTGGCCTCAACTGGCAGCTACAGCAGACTGAAAATGACACAT
TTGAGAATGGAAAAGTGAATTCTGATACCATGCCAACAAACACTGTGTCATTACCTTCTGGAGACAAT
GGAAAATTAGGTGGATTTACGCAAGAAAATAACACCATAGATTCCGGAGAACTTGATATTGGCCGAAG
AGCAATTCAAGAGATTCCTCCCGGGATCTTCTGGAGATCACAGCTCTTCATTGATCAGCCACAGTTTC
TTAAATTCAATATCTCTCTTCAGAAGGATGCATTGATTGGAGTATATGGCCGGAAAGGCTTACCGCCT
TCCCATACTCAGTATGACTTCGTGGAGCTCCTGGATGGCAGCAGGCTGATTGCCAGAGAGCAGCGGAG
CCTGCTTGAGACGGAGAGAGCCGGGCGGCAGGCGAGATCCGTCAGCCTTCATGAGGCCGGCTTTATCC
AGTACTTGGATTCTGGAATCTGGCATCTGGCTTTTTATAATGATGGGAAAAATGCAGAGCAGGTGTCT
TTTAATACCATTGTTATAGAGTCTGTGGTGGAATGTCCCCGAAATTGCCATGGAAATGGAGAATGCGT
TTCTGGAACTTGCCATTGTTTTCCAGGATTTCTGGGTCCGGATTGTTCAAGAGCCGCCTGTCCAGTGT
TATGTAGTGGCAACGGGCAGTACTCCAAGGGCCGCTGCCTGTGTTTCAGCGGCTGGAAGGGCACCGAG
TGTGATGTGCCGACTACCCAGTGTATTGACCCACAGTGTGGGGGTCGTGGGATTTGTATCATGGGCTC
TTGTGCTTGCAACTCAGGATACAAAGGAAAAAGTTGTGAAGAAGCTGACTGTATAGACCCTGGGTGTT
CTAATCATGGTGTGTGTATCCACGGGGAATGTCACTGCAGTCCAGGATGGGGAGGTAGCAATTGTGAA
ATACTGAAGACCATGTGTCCAGACCAGTGCTCCGGCCACGGAACGTATCTTCAAGAAAGTGGCTCCTG
CACGTGTGACCCTAACTGGACTGGCCCAGACTGCTCAAACGAAATATGTTCTGTGGACTGTGGCTCAC
ACGGCGTTTGCATGGGGGGGACGTGTCGCTGTGAAGAAGGCTGGACGGGCCCAGCCTGTAATCAGAGA
GCCTGCCACCCCCGCTGTGCCGAGCACGGGACCTGCAAGGATGGCAAGTGTGAATGCAGCCAGGGCTG
GAATGGAGAGCACTGCACTATCGCTCACTATTTGGATAAGATAGTTAAAGACAAGATAGGATATAAAG
AGGGTTGTCCTGGTCTGTGCAACAGCAATGGAAGATGTACCCTGGACCAAAATGGCGGACATTGTGTG
TGCCAGCCTGGATGGAGAGGAGCAGGCTGTGACGTAGCCATGGAGACTCTTTGCACAGATAGCAAGGA
CAATGAAGGGGATGGACTCATTGACTGCATGGATCCCGATTGCTGCCTACAGAGTTCCTGCCAGAATC
AGCCCTATTGTCGGGGACTGCCGGATCCTCAGGACATCATTAGCCAAAGCCTTCAATCGCCTTCTCAG
CAAGCTGCCAAATCCTTTTATGATCGAATCAGTTTCCTTATAGGATCTGATAGCACCCATGTTATACC
TGGAGAAAGTCCTTTCAATAAGAGCCTTGCATCTGTCATCAGAGGCCAAGTACTGACTGCTGATGGAA
CTCCACTTATTGGAGTAAATGTCTCGTTTTTCCATTACCCAGAATATGGATATACTATTACCCGCCAG
GACGGAATGTTTGACTTGGTGGCAAATGGTGGGGCCTCTCTAACTTTGGTATTTGAACGATCCCCATT
CCTCACTCAGTATCATACTGTGTGGATTCCATGGAATGTCTTTTATGTGATGGATACCCTAGTCATGG
AGAAAGAAGAGAATGACATTCCCAGCTGTGATCTGAGTGGATTCGTGAGGCCAAATCCCATCATTGTG
TCATCACCTTTATCCACCTTTTTCAGATCTTCTCCTGAAGACAGTCCCATCATTCCCGAAACACAGGT
ACTCCACGAGGAAACTACAATTCCAGGAACAGATTTGAAACTCTCCTACTTGAGTTCCAGAGCTGCAG
GGTATAAGTCAGTTCTCAAGATCACCATGACCCAGTCTATTATTCCATTTAATTTAATGAAGGTTCAT
CTTATGGTAGCTGTAGTAGGAAGACTCTTCCAAAAGTGGTTTCCTGCCTCACCAAACTTGGCCTATAC
TTTCATATGGGATAAAACAGATGCATATAATCAGAAAGTCTATGGTCTATCTGAAGCTGTTGTGTCAG
TTGGATATGAGTATGAGTCGTGTTTGGACCTGACTCTGTGGGAAAAGAGGACTGCCATTCTGCAGGGC
TATGAATTGGATGCGTCCAACATGGGTGGCTGGACATTAGATAAACATCACGTGCTGGATGTACAGAA
CGGTATACTGTACAAGGGAAACGGGGAAAACCAGTTCATCTCCCAGCAGCCTCCAGTCGTGAGTAGCA
TCATGGGCAATGGGCGAAGGCGCAGCATTTCCTGCCCCAGTTGCAATGGTCAAGCTGATGGTAACAAG
TTACTGGCCCCAGTGGCGCTAGCTTGTGGGATCGATGGCAGTCTGTACGTAGGCGATTTCAACTACGT
GCGGCGGATATTCCCTTCTGGAAATGTAACAAGTGTCTTAGAACTAAGAAATAAAGATTTTAGACATA
GCAGCAACCCAGCTCATAGATACTACCTTGCAACGGATCCAGTCACGGGAGATCTGTACGTTTCTGAC
ACAAACACCCGCAGAATTTATCGCCCAAAGTCACTTACGGGGGCAAAAGACTTGACTAAAAATGCAGA
AGTCGTCGCAGGGACAGGGGAGCAATGCCTTCCGTTTGACGAGGCGAGATGTGGGGATGGAGGGAAGG
CCGTGGAAGCCACACTCATGAGTCCCAAAGGAATGGCAGTTGATAAGAATGGATTAATCTACTTTGTT
GATGGAACCATGATTAGGAAAGTTGACCAAAATGGAATCATATCAACTCTTCTGGGCTCTAACGATTT
GACTTCAGCCAGACCTTTAACTTGTGACACCAGCATGCACATCAGCCAGGTACGTCTGGAATGGCCCA
CTGACCTAGCCATTAACCCTATGGATAACTCCATTTATGTCCTGGATAATAATGTAGTTTTACAGATC
ACTGAAAATCGTCAAGTTCGCATTGCTGCTGGACGGCCCATGCACTGTCAGGTTCCCGGAGTGGAATA
TCCTGTGGGGAAGCACGCGGTGCAGACAACACTGGAATCAGCCACTGCCATTGCTGTGTCCTACAGTG
GGGTCCTGTACATTACTGAAACTGATGAGAAGAAAATTAACCGGATAAGGCAGGTCACAACAGATGGA
GAAATCTCCTTAGTGGCCGGAATACCTTCAGAGTGTGACTGCAAAAATGATGCCAACTGTGACTGTTA
CCAGAGTGGAGATGGCTACGCCAAGGATGCCAAACTCAGTGCCCCATCCTCCCTGGCTGCTTCTCCAG
ATGGTACACTGTATATTGCAGATCTAGGGAATATCCGGATCCGGGCTGTGTCAAAGAATAAGCCTTTA
CTTAACTCTATGAACTTCTATGAAGTTGCGTCTCCAACTGATCAAGAACTCTACATCTTTGACATCAA
TGGTACTCACCAATATACTGTAAAGTTTAGTGCTGGTGATTACCTTTACAATTTTAGCTACAGCAATG
ACAATGATATTACTGCTGTGACAGACAGCAATGGCAACACCCTTAGAATTAGACGGGACCCAAATCGC
ATGCCAGTTCGAGTGGTGTCTCCTGATAACCAAGTGATATGGTTGACAATAGGAACAAATGGATGTTT
GAAAGGCATGACTGCTCAAGGACTGGAATTAGTTTTGTTTACTTACCATGGCAATAGTGGCCTTTTAG
CCACTAAAAGTGATGAAACTGGATGGACAACGTTTTTTGACTATGACAGTGAAGGTCGTCTGACAAAT
GTTACGTTTCCAACTGGAGTGGTCACAAACCTGCATGGGGACATGGACAAGGCTATCACAGTGGACAT
TGAGTCATCTAGCCGAGAAGAAGATGTCAGCATCACTTCAAATCTGTCCTCGATCGATTCTTTCTACA
CCATGGTTCAAGATCAGTTAAGAAACAGCTACCAGATTGGTTATGACGGCTCCCTCAGAATTATCTAC
GCCAGTGGCCTGGACTCACACTACCAAACAGAGCCGCACGTTCTGGCTGGCACCGCTAATCCGACGGT
TGCCAAAAGAAACATGACTTTGCCTGGCGAGAACGGTCAAAACTTGGTGGAATGGAGATTCCGAAAAG
AGCAAGCCCAAGGGAAAGTCAATGTCTTTGGCCGCAAGCTCAGGGTTAATGGCAGAAACCTCCTTTCA
GTTGACTTTGATCGAACAACAAAGACAGAAAAGATCTATGACGACCACCGTAAATTTCTACTGAGGAT
CGCCTACGACACGTCTGGGCACCCGACTCTCTGGCTGCCAAGCAGCAAGCTGATGGCCGTCAATGTCA
CCTATTCATCCACAGGTCAAATTGCCAGCATCCAGCGAGGCACCACTAGCGAGAAAGTAGATTATGAC
GGACAGGGGAGGATCGTGTCTCGGGTCTTTGCTGATGGTAAAACATGGAGTTACACATATTTAGAAAA
GTCCATGGTTCTTCTGCTTCATAGCCAGCGGCAGTACATCTTCGAATACGATATGTGGGACCGCCTGT
CTGCCATCACCATGCCCAGTGTGGCTCGCCACACCATGCAGACCATCCGATCCATTGGCTACTACCGC
AACATATACAACCCCCCGGAAAGCAACGCCTCCATCATCACGGACTACAACGAGGAAGGGCTGCTTCT
ACAAACAGCTTTCTTGGGTACAAGTCGGAGGGTCTTATTCAAATACAGAAGGCAGACTAGGCTCTCAG
AAATTTTATATGATAGCACAAGAGTCAGTTTTACCTATGATGAAACAGCAGGAGTCCTAAAGACAGTA
AACCTCCAGAGTGATGGTTTTATTTGCACCATTAGATACAGGCAAATTGGTCCCCTGATTGACAGGCA
GATTTTCCGCTTTAGTGAAGATGGGATGGTAAATGCAAGATTTGACTATAGCTATGACAACAGCTTTC
GAGTGACCAGCATGCAGGGTGTGATCAATGAAACGCCACTGCCTATTGATCTGTATCAGTTTGATGAC
ATTTCTGGCAAAGTTGAGCAGTTTGGAAAGTTTGGAGTTATATATTATGATATTAACCAGATCATTTC
TACAGCTGTAATGACCTATACGAAGCACTTTGATGCTCATGGCCGTATCAAGGAGATTCAATATGAGA
TATTCAGGTCGCTCATGTACTGGATTACAATTCAGTATGATAACATGGGTCGGGTAACCAAGAGAGAG
ATTAAAATAGGGCCCTTTGCCAACACCACCAAATATGCTTATGAATATGATGTTGATGGACAGCTCCA
AACAGTTTACCTCAATGAAAAGATAATGTGGCGGTACAACTACGATCTGAATGGAAACCTCCATTTAC
TGAACCCAAGTAACAGTGCGCGTCTGACACCCCTTCGCTATGACCTGCGAGACAGAATCACTCGACTG
GGTGATGTTCAATATCGGTTGGATGAAGATGGTTTCCTACGTCAAAGGGGCACGGAAATCTTTGAATA
TAGCTCCAAGGGGCTTCTAACTCGAGTTTACAGTAAAGGCAGTGGCTGGACAGTGATCTACCGTTATG
ACGGCCTGGGAAGGCGTGTTTCTAGCAAAACCAGTCTAGGACAGCACCTGCAGTTTTTTTATGCTGAC
TTAACTTATCCCACTAGGATTACTCATGTCTACAACCATTCGAGTTCAGAAATTACCTCCCTGTATTA
TGATCTCCAAGGACATCTTTTTGCCATGGAAATCAGCAGTGGGGATGAATTCTATATTGCATCGGATA
ACACAGGGACACCACTGGCTGTGTTCAGTAGCAATGGGCTTATGCTGAAACAGATTCAGTACACTGCA
TATGGGGAAATCTATTTTGACTCTAATATTGACTTTCAACTGGTAATTGGATTTCATGGTGGCCTGTA
TGACCCACTCACCAAATTAATCCACTTTGGAGAAAGAGATTATGACATTTTGGCAGGACGGTGGACAA
CACCTGACATAGAAATCTGGAAAAGAATTGGGAAGGACCCAGCTCCTTTTAACTTGTACATGTTTAGG
AATAACAACCCTGCAAGCAAAATCCATGACGTGAAAGATTACATCACAGATGTTAACAGCTGGCTGGT
GACATTTGGTTTCCATCTGCACAATGCTATTCCTGGATTCCCTGTTCCCAAATTTGATTTAACAGAAC
CTTCTTACGAACTTGTGAAGAGTCAGCAGTGGGATGATATACCGCCCATCTTCGGAGTCCAGCAGCAA
GTGGCGCGGCAGGCCAAGGCCTTCCTGTCGCTGGGGAAGATGGCCGAGGTGCAGGTGAGCCGGCGCCG
GGCCGGCGGCGCGCAGTCCTGGCTGTGGTTCGCCACGGTCAAGTCGCTGATCGGCAAGGGCGTCATGC
TGGCCGTCAGCCAGGGCCGCGTGCAGACCAACGTGCTCAACATCGCCAACGAGGACTGCATCAAGGTG
GCGGCCGTGCTCAACAACGCCTTCTACCTGGAGAACCTGCACTTCACCATCGAGGGCAAGGACACGCA
CTACTTCATCAAGACCACCACGCCCGAGAGCGACCTGGGCACGCTGCGGTTGACCAGCGGCCGCAAGG
CGCTGGAGAACGGCATCAACGTGACGGTGTCGCAGTCCACCACGGTGGTGAACGGCAGGACGCGCAGG
TTCGCGGACGTGGAGATGCAGTTCGGCGCGCTGGCGCTGCACGTGCGCTACGGCATGACCCTGGACGA
GGAGAAGGCGCGCATCCTGGAGCAGGCGCGGCAGCGCGCGCTCGCCCGGGCCTGGGCGCGCGAGCAGC
AGCGCGTGCGCGACGGCGAGGAGGGCGCGCGCCTCTGGACGGAGGGCGAGAAGCGGCAGCTGCTGAGC
GCCGGCAAGGTGCAGGGCTACGACGGGTACTACGTACTCTCGGTGGAGCAGTACCCCGAGCTGGCCGA
CAGCGCCAACAACATCCAGTTCCTGCGGCAGAGCGAGATCGGCAGGAGGTAACGCCCGGGCCGCGCCC
GCCGAGCCGCTCACGCCCTGCCCACATTGTCCTGTGGCACAACCCGAGTGGGACTCTCCAACGCCCAA
GAGCCTTCCTCCCGGGGGAATGAGACTGCTGTTACGACCCACACCCACACCGCGAAAACAAGGACCGC
TTTTTTCCGAATGACCTTAAAGGTGATCGGCTTTAACGAATATGTTTACATATGCATAGCGCTGCACT
CAGTCGGACTGAACGTAGCCAGAGGAAAAAAAAATCATCAAGGACAAAGGCCTCGACCTGTTGCGCTG
GGCCGTCTGTTCCTTCTAGGCACTGTATTTAACTAACTTTA

| | | | |
|---|---|---|---|
| NOV40i, CG55069-03 | SEQ ID NO: 502 | 2628 aa | MW at 293503.3kD |
| Protein Sequence | | | |

MDVKERRPYCSLTKSRREKERRYTNSSADNEECRVPTQKSYSSSETLKAFDHDSSRLLYGNRVKDLVH
READEFTRQEQPASNQGQSTLQPLPPSHKQHSAQHHPSITSLNRNSLTNRRNQSPAPPAALPAELQTT
PESVQLQDSWVLGSNVPLESRHFLFKTGTGTTPLFSTATPGYTMASGSVYSPPTRPLPRNTLSRSAFK
FKKSSKYCSWKCTALCAVGVSVLLAILLSYFIANHLFGLNWQLQQTENDTFENGKVNSDTMPTNTVSL
PSGDNGKLGGFTQENNTIDSGELDIGRRAIQEIPPGIFWRSQLFIDQPQFLKFNISLQKDALIGVYGR
KGLPPSHTQYDFVELLDGSRLIAREQRSLLETERAGRQARSVSLHEAGFIQYLDSGIWHLAFYNDGKM
AEQVSFNTIVIESVVECPRNCHGNGECVSGTCHCFPGFLGPDCSRAACPVLCSGNGQYSKGRCLCFSG
WKGTECDVPTTQCIDPQCGGRGICIMGSCACNSGYKGKSCEEADCIDPGCSNHGVCIHGECHCSPGWG
GSNCEILKTMCPDQCSGHGTYLQESGSCTCDPNWTGPDCSNEICSVDCGSHGVCMGGTCRCEEGWTGP
ACNQRACHPRCAEHGTCKDGKCECSQGWNGEHCTIAHYLDKIVKDKIGYKEGCPGLCNSNGRCTLDQN
GGHCVCQPGWRGAGCDVANETLCTDSKDNEGDGLIDCMDPDCCLQSSCQNQPYCRGLPDPQDIISQSL
QSPSQQAAKSFYDRISFLIGSDSTHVIPGESPFNKSLASVIRGQVLTADGTPLIGVNVSFFHYPEYGY
TITRQDGMFDLVANGGASLTLVFERSPFLTQYHTVWIPWNVFYVMDTLVMEKEENDIPSCDLSGFVRP
NPIIVSSPLSTFFRSSPEDSPIIPETQVLHEETTIPGTDLKLSYLSSRAAGYKSVLKITMTQSIIPFN
LMKVHLMVAVVGRLFQKWFPASPNLAYTFIWDKTDAYNQKVYGLSEAVVSVGYEYESCLDLTLWEKRT
AILQGYELDASNNGGWTLDKHHVLDVQNGILYKGNGENQFISQQPPVVSSIMGNGRRRSISCPSCNGQ
ADGNKLLAPVALACGIDGSLYVGDFNYVRRIFPSGNVTSVLELRNKDFRHSSNPAHRYYLATDPVTGD
LYVSDTNTRRIYRPKSLTGAKDLTKNAEVVAGTGEQCLPFDEARCGDGGKAVEATLMSPKGMAVDKNG
LIYFVDGTMIRKVDQNGIISTLLGSNDLTSARPLTCDTSMHISQVRLEWPTDLAINPMDNSIYVLDNN
VVLQITENRQVRIAAGRPMHCQVPGVEYPVGKHAVQTTLESATAIAVSYSGVLYITETDEKKINRIRQ
VTTDGEISLVAGIPSECDCKNDANCDCYQSGDGYAKDAKLSAPSSLAASPDGTLYIADLGNIRIRAVS
KNKPLLNSMNFYEVASPTDQELYIFDINGTHQYTVSLVTGDYLYNFSYSNDNDITAVTDSNGNTLRIR
RDPNRMPVRVVSPDNQVIWLTIGTNGCLKGMTAQGLELVLFTYHGNSGLLATKSDETGWTTFFDYDSE
GRLTNVTFPTGVVTNLHGDMDKAITVDIESSSREEDVSITSNLSSIDSFYTMVQDQLRNSYQIGYDGS
LRIIYASGLDSHYQTEPHVLAGTANPTVAKRNMTLPGENGQNLVEWRFRKEQAQGKVNVFGRKLRVNG
RNLLSVDFDRTTKTEKIYDDHRKFLLRIAYDTSGHPTLWLPSSKLMAVNVTYSSTGQIASIQRGTTSE
KVDYDGQGRIVSRVFADGKTWSYTYLEKSMVLLLHSQRQYIFEYDMWDRLSAITMPSVARHTMQTIRS
IGYYRNIYNPPESNASIITDYNEEGLLLQTAFLGTSRRVLFKYRRQTRLSEILYDSTRVSFTYDETAG
VLKTVNLQSDGFICTIRYRQIGPLIDRQIFRFSEDGMVNARFDYSYDNSFRVTSMQGVINETPLPIDL
YQFDDISGKVEQFGKFGVIYYDINQIISTAVMTYTKHFDAHGRIKEIQYEIFRSLMYWITIQYDNMGR
VTKREIKIGPFANTTKYAYEYDVDGQLQTVYLNEKIMWRYNYDLNGNLHLLNPSMSARLTPLRYDLRD
RITRLGDVQYRLDEDGFLRQRGTEIFEYSSKGLLTRVYSKGSGWTVIYRYDGLGRRVSSKTSLGQHLQ
FFYADLTYPTRITHVYNHSSSEITSLYYDLQGHLFAMEISSGDEFYIASDNTGTPLAVFSSNGLMLKQ
IQYTAYGEIYFDSNIDFQLVIGFHGGLYDPLTKLIHFGERDYDILAGRWTTPDIEIWKRIGKDPAPFN
LYMFRNNNPASKIHDVKDYITDVNSWLVTFGFHLHNAIPGFPVPKFDLTEPSYELVKSQQWDDIPPIF
GVQQQVARQAKAFLSLGKMAEVQVSRRRAGGAQSWLWFATVKSLIGKGVMLAVSQGRVQTNVLNIANE
DCIKVAAVLNWAFYLENLHFTIEGKDTHYFIKTTTPESDLGTLRLTSGRKALENGINVTVSQSTTVVN
GRTRRFADVEMQFGALALHVRYGMTLDEEKARILEQARQRALARAWAREQQRVRDGEEGARLWTEGEK
RQLLSAGKVQGYDGYYVLSVEQYPELADSANNIQFLRQSEIGRR

| | | |
|---|---|---|
| NOV40j, 219937039 | SEQ ID NO: 503 | 1854 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGAAATAAAGATTTTAGACATAGCAGCAACCCAGCTCATAGATACTACCTTGCAACGGAT
CCAGTCACGGGAGATCTGTACGTTTCTGACACAAACACCCGCAGAATTTATCGCCCAAAGTCACTTAC
GGGGGCAAAAGACTTGACTAAAAATGCAGAAGTCGTCGCAGGGACAGGGGAGCAATGCCTTCCGTTTG
ACGAGGCGAGATGTGGGGATGGAGGGAAGGCCGTGGAAGCCACACTCATGAGTCCCAAAGGAATGGCA
GTTGATAAGAATGGATTAATCTACTTTGTTGATGGAACCATGATTAGGAAAGTTGACCAAAATGGAAT
CATATCAACTCTTCTGGGTTCTAACGATTTGACTTCAGCCAGACCTTTAACTTGTGACACCAGCATGC
ACATCAGCCAGGTACGTCTGGAATGGCCCACTGACCTAGCCATTAACCCTATGGATAACTCCATTTAT
GTCCTGGATAATGTCGAC

| | | | |
|---|---|---|---|
| NOV40j, 219937039 | SEQ ID NO: 504 | 618 aa | MW at 68016.2kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVANGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPNLAYTFIWDKTDAYNQKVYGLSEAVVSVGYEYESCLDLTL
WEKRTAILQGYELDASNMGGWTLDKMHVLDVQNGILYKGNGENQFISQQPPVVSSIMGNGRRRSISCP
SCNGQADGNKLLAPVALACGIDGSLYVGDFNYVRRIFPSGNVTSVLELRNKDFRHSSNPAHRYYLATD
PVTGDLYVSDTNTRRIYRPKSLTGAKDLTKNAEVVAGTGEQCLPFDEARCGDGGKAVEATLMSPKGMA
VDKNGLIYFVDGTMIRKVDQNGIISTLLGSNDLTSARPLTCDTSMHISQVRLEWPTDLAINPMDNSIY
VLDNVD

| | | |
|---|---|---|
| NOV40k, 219937046 | SEQ ID NO: 505 | 1854 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGTTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAGTGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGCGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGAAATAAAGATTTTAGACATAGCAGCAACCCAGCTCATAGATACTACCTTGCAACGGAT
CCAGTCACGGGAGATCTGTACGTTTCTGACACAAACACCCGCAGAATTTATCGCCCAAAGTCACTTAC
GGGGGCAAAAGACTCGACTAAAAATGCAGAAGTCGTCGCAGGGACAGGGGAGCAATGCCTTCCGTTTG
ACGAGGCGAGATGTGGGGATGGAGGGAAGGCCGTGGAAGCCACACTCATGAGTCCCAAAGGAATGGCA
GTTGATAAGAATGGATTAATCTACTTTGTTGATGGAACCATGATTAGGAAAGTTGACCAAAATGGAAT
CATATCAACTCTTCTGGGTTCTAACGATTTGACTTCAGCCAGACCTTTAACTTGTGACACCAGCATGC
ACATCAGCCAGGTACGTCTGGAATGGCCCACTGACCTAGCCATTAACCCTATGGATAACTCCATTTAT
GTCCTGGATAATGTCGAC

| | | | |
|---|---|---|---|
| NOV40k, 219937046 | SEQ ID NO: 506 | 618 aa | MW at 67935.1kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVASGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPNLAYTFIWDKTDAYNQKVYGLSEAVVSVGYEYESCLDLTL
WEKRTAILQGYELDASNMGGWTLDKHHALDVQNGILYKGNGENQFISQQPPVVSSINGNGRRRSISCP
SCNGQADGNKLLAPVALACGIDGSLYVGDFNYVRRIFPSGNVTSVLELRNKDFRHSSNPAHRYYLATD
PVTGDLYVSDTNTRRIYRPKSLTGAKDSTKNAEVVAGTGEQCLPFDEARCGDGGKAVEATLMSPKGMA
VDKNGLIYFVDGTMIRKVDQNGIISTLLGSNDLTSARPLTCDTSMHISQVRLEWPTDLAINPMDNSIY
VLDNVD

| | | |
|---|---|---|
| NOV40l, 219937583 | SEQ ID NO: 507 | 1834 bp |
| DNA Sequence | ORF Start: at 2 | ORF Stop: end of sequence |

TAAGCTTGACCAAAATGGCGGACATTGTGTGTGCCAGCCTGGATGGAGAGGAGCAGGCTGTGACGTAG
CCATGGAGACTCTTTGCACAGATAGCAAGGACAATGAAGGAGATGGACTCATTGACTGCATGGATCCC
GATTGCTGCCTACAGAGTTCCTGCCAGAATCAGCCCTATTGTCGGGGACTGCCGGATCCTCAGGACAT
CATTAGCCAAAGCCTTCAATCGCCTTCTCAGCAAGCTGCCAAATCCTTTTATGATCGAATCAGTTTCC
TTATAGGATCTGATAGCACCCATGTTATACCTGGAGAAAGTCCTTTCAATAAGAGCCTTGCATCTGTC
ATCAGAGGCCAAGTACTGACTGCTGATGGAACTCCACTTATTGGAGTAAATGTCTCGTTTTTCCATTA
CCCAGAATATGGATATACTATTACCCGCCAGGACGGAATGTTTGACTTGGTGGCAAATGGTGGGGCCT
CTCTAACTTTGGTATTTGAACGATCCCCATTCCTCACTCAGTATCATACTGTGTGGATTCCATGGAAT
GTCTTTTATGTGATGGATACCCTAGTCATGAAGAAAGAAGAGAATGACATTCCCAGCTGTGATCTGAG
TGGATTCGTGAGGCCAAATCCCATCATTGTGTCATCACCTTTATCCACCTTTTTCAGATCTTCTCCTG
AAGACAGTCCCATCATTCCCGAAACACAGGTACTCCACGAGGAAACTACAATTCCAGGAACAGATTTG
AAACTCTCCTACTTGAGTTCCAGAGCTGCAGGGTATAAGTCAGTTCTCAAGATCACCATGACCCAGTC
TATTATTCCATTTAATTTAATGAAGGTTCATCTTATGGTAGCTGTAGTAGGAAGACTCTTCCAAAAGT
GGTTTCCTGCCTCACCAAACTTGGCCTATACTTTCATATGGGATAAAACAGATGCATATAATCAGAAA
GTCTATGGTCTATCTGAAGCTGTTGTGTCAGTTGGATATGAGTATGAGTCGTGTTTGGACCTGACTCT
GTGGGAAAAGAGGACTGCCATTCTGCAGGGCTATGAATTGGATGCGTCCAACATGGGTGGCTGGACAT
TAGATAAACATCACGTGCTGGATGTACAGAACGGTATACTGTACAAGGGAAACGGGGAAAACCAGTTC
ATCTCCCAGCAGCCTCCAGTCGTGAGTAGCATCATGGGCAATGGGCGAAGGCGCAGCATTTCCTGCCC
CAGTTGCAATGGTCAAGCTGATGGTAACAAGTTACTGGCCCCAGTGGCGCTAGCTTGTGGGATCGATG
GCAGTCTGTACGTAGGCGATTTCAACTATGTGCGGCGGATATTCCCTTCTGGAAATGTAACAAGTGTC
TTAGAACTAAGCAGCAACCCAGCTCATAGATACTACCTTGCAACGGATCCAGTCACGGGAGATCTGTA
CGTTTCTGACACAAACACCCGCAGAATTTATCGCCCAAAGTCACTTACGGGGGCAAAAGACTTGACTA
AAAATGCAGAAGTCGTCGCAGGGACAGGGGAGCAATGCCTTCCGTTTGACGAGGCGAGATGTGGGGAT
GGAGGGAAGGCCGTGGAAGCCACACTCATGAGTCCCAAAGGAATGGCAGTTGATAAGAATGGATTAAT
CTACTTTGTTGATGGAACCATGATTAGGAAAGTTGACCAAAATGGAATCATATCAACTCTTCTGGGTT
CTAACGATTTGACTTCAGCCAGACCTTTAACTTGTGACACCAGCATGCACATCAGCCAGGTACGTCTG
GAATGGCCCACTGACCTAGCCATTAACCCTATGGATAACTCCATTTATGTCCTGGATAATGTCGAC

| | | | |
|---|---|---|---|
| NOV40l, 219937583 | SEQ ID NO: 508 | 611 aa | MW at 67062.2kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVASGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPNLAYTFIWDKTDAYNQKVYGLSEAVVSVGYEYESCLDLTL
WEKRTAILQGYELDASNMGGWTLDKHHALDVQNGILYKGNGENQFISQQPPVVSSINGNGRRRSISCP
SCNGQADGNKLLAPVALACGIDGSLYVGDFNYVRRIFPSGNVTSVLELRNKDFRHSSNPAHRYYLATD
PVTGDLYVSDTNTRRIYRPKSLTGAKDSTKNAEVVAGTGEQCLPFDEARCGDGGKAVEATLMSPKGMA
VDKNGLIYFVDGTMIRKVDQNGIISTLLGSNDLTSARPLTCDTSMHISQVRLEWPTDLAINPMDNSIY
VLDNVD

| | | |
|---|---|---|
| NOV40m, 219937000 | SEQ ID NO: 509 | 1833 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTTTAACTTGTGACACCAGCATGCACATCAGCCAGGTACGTCTGG
AATGGCCCACTGACCTAGCCATTAACCCTATGGATAACTCCATTTATGTCCTGGATAATGTCGAC

| | | | |
|---|---|---|---|
| NOV40m, 219937000 | SEQ ID NO: 510 | 611 aa | MW at 67062.2kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVANGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPMLAYTFIWDKTDAYNQKVYGLSEAVVSVGYEYESCLDLTL
WEKRTAILQGYELDASNMGGWTLDKHHVLDVQNGILYKGNGENQFISQQPPVVSSIMGNGRRRSISCP
SCNGQADGNKLLAPVAIACGIDGSLYVGDFNYVRRIFPSGNVTSVLELSSNPAHRYYLATDPVTGDLY
VSDTNTRRIYRPKSLTGAKDLTKNAEVVAGTGEQCLPFDEARCGDGGKAVEATLMSPKGMAVDKNGLI
YFVDGTMIRKVDQNGIISTLLGSNDLTSARPLTCDTSMHISQVRLEWPTDLAINPMDNSIYVLDNVD

| | | |
|---|---|---|
| NOV40n, 219937005 | SEQ ID NO: 511 | 1833 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTTTAACTTGTGACACCAGCATGCACATCAGCCAGGTACGTCTGG
AATGGCCCACTGACCTAGCCATTAACCCTATGGATAACTCCATTTATGTCCTGGATAATGTCGAC

| | | | |
|---|---|---|---|
| NOV40n, 219937005 | SEQ ID NO: 512 | 611 aa | MW at 67062.2kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVANGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPMLAYTFIWDKTDAYNQKVYGLSEAVVSVGYEYESCLDLTL
WEKRTAILQGYELDASNMGGWTLDKHHVLDVQNGILYKGNGENQFISQQPPVVSSIMGNGRRRSISCP
SCNGQADGNKLLAPVAIACGIDGSLYVGDFNYVRRIFPSGNVTSVLELSSNPAHRYYLATDPVTGDLY
VSDTNTRRIYRPKSLTGAKDLTKNAEVVAGTGEQCLPFDEARCGDGGKAVEATLMSPKGMAVDKNGLI
YFVDGTMIRKVDQNGIISTLLGSNDLTSARPLTCDTSMHISQVRLEWPTDLAINPMDNSIYVLDNVD

| | | |
|---|---|---|
| NOV40o, 219937013 | SEQ ID NO: 513 | 1833 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTTTAACTTGTGACACCAGCATGCACATCAGCCAGGTACGTCTGG
AATGGCCCACTGACCTAGCCATTAACCCTATGGATAACTCCATTTATGTCCTGGATAATGTCGAC

| | | | |
|---|---|---|---|
| NOV40o, 219937013 | SEQ ID NO: 514 | 611 aa | MW at 67034.1kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVANGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPMLAYTFIWDKTDAYNQKVYGLSEAVVSVGYEYESCLDLTL
WEKRTAILQGYELDASNMGGWTLDKHHVLDVQNGILYKGNGENQFISQQPPVVSSIMGNGRRRSISCP
SCNGQADGNKLLAPVAIACGIDGSLYVGDFNYVRRIFPSGNVTSVLELSSNPAHRYYLATDPVTGDLY
VSDTNTRRIYRPKSLTGAKDLTKNAEVVAGTGEQCLPFDEARCGDGGKAVEATLMSPKGMAVDKNGLI
YFVDGTMIRKVDQNGIISTLLGSNDLTSARPLTCDTSMHISQVRLEWPTDLAINPMDNSIYVLDNVD

| | | |
|---|---|---|
| NOV40p,219937063 | SEQ ID NO: 515 | 1833 bp |
| DNA Sequence | ORF Start: at 1 | ORF Stop: end of sequence |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTTTAACTTGTGACACCAGCATGCACATCAGCCAGGTACGTCTGG
AATGGCCCACTGACCTAGCCATTAACCCTATGGATAACTCCATTTATGTCCTGGATAATGTCGAC

| | | | |
|---|---|---|---|
| NOV40p, 219937063 | SEQ ID NO: 516 | 611 aa | MW at 67026.1kD |
| Protein Sequence | | | |

KLDQNGGHCVCQPGWRGAGCDVAMETLCTDSKDNEGDGLIDCMDPDCCLQSSCQNQPYCRGLPDPQDI
ISQSLQSPSQQAAKSFYDRISFLIGSDSTHVIPGESPFNKSLASVIRGQVLTADGTPLIGVNVSFFHY
PEYGYTITRQDGMFDLVANGGASLTLVFERSPFLTQYHTVWIPWNVFYVMDTLVMKKEENDIPSCDLS
GFVRPNPIIVSSPLSTFFRSSPEDSPIIPETQVLHEETTIPGTDLKLSYLSSRAAGYKSVLKITMTQS
IIPFNLMKVHLMVAVVGRLFQKWFPASPMLAYTFIWDKTDAYNQKVYGLSEAVVSVGYEYESCLDLTL
WEKRTAILQGYELDASNMGGWTLDKHHVLDVQNGILYKGNGENQFISQQPPVVSSIMGNGRRRSISCP
SCNGQADGNKLLAPVAIACGIDGSLYVGDFNYVRRIFPSGNVTSVLELSSNPAHRYYLATDPVTGDLY
VSDTNTRRIYRPKSLTGAKDLTKNAEVVAGTGEQCLPFDEARCGDGGKAVEATLMSPKGMAVDKNGLI
YFVDGTMIRKVDQNGIISTLLGSNDLTSARPLTCDTSMHISQVRLEWPTDLAINPMDNSIYVLDNVD

| | | |
|---|---|---|
| NOV40q, GG55069-09 | SEQ ID NO: 517 | 2536 bp |
| DNA Sequence | ORF Start: at 7 | ORF Stop: at 2470 |

GGTACCAACTGGCAGCTACAGCAGACTGAAAATGACACATTTGAGAATGGAAAAGTGAATTCTGATAC
CATGCCAACAAACACTGTGTCATTACCTTCTGGAGACAATGGAAAATTAGGTGGATTTACGCAAGAAA
ATAACACCATAGATTCCGGAGAACTTGATATTGGCCGAAGAGCAATTCAAGAGATTCCTCCCGGGATC
TTCTGGAGATCACAGCTCTTCATTGATCAGCCACAGTTTCTTAAATTCAATATCTCTCTTCAGAAGGA
TGCATTGATTGGAGTATATGGCCGGAAAGGCTTACCGCCTTCCCATACTCAGTATGACTTCGTGGAGC
TCCTGGATGGCAGCAGGCTGATTGCCAGAGAGCAGCGGAGCCTGCTTGAGACGGAGAGAGCCGGGCGG
CAGGCGAGATCCGTCAGCCTTCATGAGGCCGGCTTTATCCAGTACTTGGATTCTGGAATCTGGCATCT
GGCTTTTTATAATGATGGGAAAAATGCAGAGCAGGTGTCTTTTAATACCATTGTTATAGAGTCTGTGG
TGGAATGTCCCCGAAATTGCCATGGAAATGGAGAATGCGTTTCTGGAACTTGCCATTGTTTTCCAGGA
TTTCTGGGTCCGGATTGTTCAAGAGCCGCCTGTCCAGTGTTATGTAGTGGCAACGGGCAGTACTCCAA
GGGCCGCTGCCTGTGTTTCAGCGGCTGGAAGGGCACCGAGTGTGATGTGCCGACTACCCAGTGTATTG
ACCCACAGTGTGGGGGTCGTGGGATTTGTATCATGGGCTCTTGTGCTTGCAACTCAGGATACAAAGGA
GAAAGTTGTGAAGAAGCTGACTGTATAGACCCTGGGTGTTCTAATCATGGTGTGTGTATCCACGGGGA
ATGTCACTGCAGTCCAGGATGGGGAGGTAGCAATTGTGAAATACTGAAGACCATGTGTCCAGACCAGT
GCTCCGGCCACGGAACGTATCTTCAAGAAAGTGGCTCCTGCACGTGTGACCCTAACTGGACTGGCCCA
GACTGCTCAAACGAAATATGTTCTGTGGACTGTGGCTCACACGGCGTTTGCATGGGGGGGACGTGTCG
CTGTGAAGAAGGCTGGACGGGCCCAGCCTGTAATCAGAGAGCCTGCCACCCCCGCTGTGCCGAGCACG
GGACCTGCAAGGATGGCAAGTGTGAATGCAGCCAGGGCTGGAATGGAGAGCACTGCACTATCGCTCAC
TATTTGGATAAGATAGTTAAAGAGGGTTGTCCTGGTCTGTGCAACAGCAATGGAAGATGTACCCTGGA
CCAAAATGGCTGGCATTGTGTGTGCCAGCCTGGATGGAGAGGAGCAGGCTGTGACGTAGCCATGGAGA
CTCTTTGCACAGATAGTAAGGACAATGAAGGAGATGGACTCATTGACTGCATGGATCCCGATTGCTGC
CTACAGAGTTCCTGCCAGAATCAGCCCTATTGTCGGGGACTGCCGGATCCTCAGGACATCATTAGCCA
AAGCCTTCAATCGCCTTCTCAGCAAGCTGCCAAATCCTTTTATGATCGAATCAGTTTCCTTATAGGAT
CTGATAGCACCCATGTTATACCTGGAGAAAGTCCTTTCAATAAGAGCCTTGCATCTGTCATCAGAGGC
CAAGTACTGACTGCTGATGGAACTCCACTTATTGGAGTAAATGTCTCGTTTTTCCATTACCCAGAATA
TGGATATACTATTACCCGCCAGGACGGAATGTTTGACTTGGTGGCAAATGGTGGGGCCTCTCTAACTT
TGGTATTTGAACGATCCCCATTCCTCACTCAGTATCATACTGTGTGGATTCCATGGAATGTCTTTTAT
GTGATGGATACCCTAGTCATGAAGAAAGAAGAGAATGACATTCCCAGCTGTGATCTGAGTGGATTCGT
GAGGCCAAATCCCATCATTGTGTCATCACCTTTATCCACCTTTTTCAGATCTTCTCCTGAAGACAGTC
CCATCATTCCCGAAACACAGGTACTCCACGAGGAAACTACAATTCCAGGAACAGATTTGAAACTCTCC
TACTTGAGTTCCAGAGCTGCAGGGTATAAGTCAGTTCTCAAGATCACCATGACCCAGTCTATTATTCC
ATTTAATTTAATGAAGGTTCATCTTATGGTAGCTGTAGTAGGAAGACTCTTCCAAAAGTGGTTTCCTG
CCTCACCAAACTTGGCCTATACTTTCATATGGGATAAAACAGATGCATATAATCAGAAAGTCTATGGT
CTATCTGAAGCTGTTGTGTCAGTTGGATATGAGTATGAGTCGTGTTTGGACCTGACTCTGTGGGAAAA
GAGGACTGCCATTCTGCAGGGCTATGAATTGGATGCGTCCAACATGGGTGGCTGGACATTAGATAAAC
ATCACGTGCTGGATGTACAGAACGGTATACTGTACAAGGGAAACGGGGAAAACCAGTTCATCTCCCAG
CAGCCTCCAGTCGTGAGTAGCGTCGACGCTACACACGACTGGAGGCTGCTGGTCATTTTCAGCTCTGC
AGTAGCTGCCAGTTGGTACC

| | | | |
|---|---|---|---|
| NOV40q, CG55069-09 | SEQ ID NO: 518 | 821 aa | MW at 89886.1kD |
| Protein Sequence | | | |

NWQLQQTENDTFENGKVNSDTMPTNTVSLPSGDNGKLGGFTQENNTIDSGELDIGRRAIQEIPPGIFW
RSQLFIDQPQFLKFNISLQKDALIGVYGRKGLPPSHTQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACNSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKEGCPGLCNSNGRCTLDQNGWHCVCQPGWRGAGCDVAMETLCTDSKDNEGDGLIDCMDPDCCLQ
SSCQNQPYCRGLPDPQDIISQSLQSPSQQAAKSFYDRISFLIGSDSTHVIPGESPFNKSLASVIRGQV
LTADGTPLIGVNVSFFHYPEYGYTITRQDGMFDLVANGGASLTLVFERSPFLTQYHTVWIPWNVFYVM
DTLVMKKEENDIPSCDLSGFVRPNPIIVSSPLSTFFRSSPEDSPIIPETQVLHEETTIPGTDLKLSYL
SSRAAGYKSVLKITMTQSIIPFNLMKVHLMVAVVGRLFQKWFPASPNLAYTFIWDKTDAYNQKVYGLS
EAVVSVGYEYESCLDLTLWEKRTAILQGYELDASNMGGWTLDKHHVLDVQNGILYKGNGENQFISQOP
PVVSS

| | | |
|---|---|---|
| NOV40r, 309327410 | SEQ ID NO: 519 | 2482 bp |
| DNA Sequence | ORF Start: at 2 | ORF Stop: end of sequence |

CACCTCGCGAAACTGGCAGCTACAGCAGACTGAAAATGACACATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGGCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGGCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCATTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGGGCACCGAGTGTGATGTGCCGACTACCCAGTGT
ATTGACCCACAGTGTGGGGGTCGTGGGATTTGTATCATGGGCTCTTGTGCTTGCAACTCAGGATACAA
AGGAGAAAGTTGTGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGTGCTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GTCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGAGGGTTGTCCTGGTCTGTGCAACAGCAATGGAAGATGTACCC
TGGACCAAAATGGCTGGCATTGTGTGTGCCAGCCTGGATGGAGAGGAGCAGGCTGTGACGTAGCCATG
GAGACTCTTTGCACAGATAGTAAGGACAATGAAGGAGATGGACTCATTGACTGCATGGATCCCGATTG
CTGCCTACAGAGTTCCTGCCAGAATCAGCCCTATTGTCGGGGACTGCCGGATCCTCAGGACATCATTA
GCCAAAGCCTTCAATCGCCTTCTCAGCAAGCTGCCAAATCCTTTTATGATCGAATCAGTTTCCTTATA
GGATCTGATAGCACCCATGTTATACCTGGAGAAAGTCCTTTCAATAAGAGCCTTGCATCTGTCATCAG
AGGCCAAGTACTGACTGCTGATGGAACTCCACTTATTGGAGTAAATGTCTCGTTTTTCCATTACCCAG
AATATGGATATACTATTACCCGCCAGGACGGAATGTTTGACTTGGTGGCAAATGGTGGGGCCTCTCTA
ACTTTGGTATTTGAACGATCCCCATTCCTCACTCAGTATCATACTGTGTGGATTCCATGGAATGTCTT
TTATGTGATGGATACCCTAGTCATGAAGAAAGAAGAGAATGACATTCCCAGCTGTGATCTGAGTGGAT
TCGTGAGGCCAAATCCCATCATTGTGTCATCACCTTTATCCACCTTTTTCAGATCTTCTCCTGAAGAC
AGTCCCATCATTCCCGAAACACAGGTACTCCACGAGGAAACTACAATTCCAGGAACAGATTTGAAACT
CTCCTACTTGAGTTCCAGAGCTGCAGGGTATAAGTCAGTTCTCAAGATCACCATGACCCAGTCTATTA
TTCCATTTAATTTAATGAAGGTTCATCTTATGGTAGCTGTAGTAGGAAGACTCTTCCAAAAGTGGTTT
CCTGCCTCACCAAACTTGGCCTATACTTTCATATGGGATAAAACAGATGCATATAATCAGAAAGTCTA
TGGTCTATCTGAAGCTGTTGTGTCAGTTGGATATGAGTATGAGTCGTGTTTGGACCTGACTCTGTGGG
AAAAGAGGACTGCCATTCTGCAGGGCTATGAATTGGATGCGTCCAACATGGGTGGCTGGACATTAGAT
AAACATCACGTGCTGGATGTACAGAACGGTATACTGTACAAGGGAAACGGGGAAAACCAGTTCATCTC
CCAGCAGCCTCCAGTCGTGAGTAGCCTCGAGGGC

| | | | |
|---|---|---|---|
| NOV40r, 309327410 | SEQ ID NO: 520 | 827 aa | MW at 90529.8kD |
| Protein Sequence | | | |

TSRNWQLQQTENDTFENGKVNSDTMPTNTVSLPSGDNGKIGGFTQENNTIDSGELDIGRRAIQEIPPG
IFWRSQLFIDQPQFLKFNISLQKDALIGVYGRKGLPPSHTQYDFVELLDGSRLIAREQRSLLETERAG
RQARSVSLHEAGFIQYLDSGIWHLAFYNDGKNAEQVSFNTIVIESVVECPRNCHGNGECVSGTCHCFP
GFLGPDCSRAACPVLCSGNGQYSKGRCLCFSGWKGTECDVPTTQCIDPQCGGRGICIMGSCACNSGYK
GESCEEADCIDPGCSNHGVCIHGECHCSPGWGGSNCEILKTMCPDQCSGHGTYLQESGSCTCDPNWTG
PDCSNEICSVDCGSHGVCMGGTCRCEEGWTGPACNQRACHPRCAEHGTCKDGKCECSQGWNGEHCTIA
HYLDKIVKEGCPGLCNSNGRCTLDQNGWHCVCQPGWRGAGCDVAMETLCTDSKDNEGDGLIDCMDPDC
CLQSSCQNQPYCRGLPDPQDIISQSLQSPSQQAAKSFYDRISFLIGSDSTHVIPGESPFNKSLASVIR
GQVLTADGTPLIGVNVSFFHYPEYGYTITRQDGMFDLVANGGASLTLVFERSPFLTQYHTVWIPWNVF
YVMDTLVMKKEENDIPSCDLSGFVRPNPIIVSSPLSTFFRSSPEDSPIIPETQVLHEETTIPGTDLKL
SYLSSRAAGYKSVLKITMTQSIIPFNLMKVHLMVAVVGRLFQKWFPASPNLAYTFIWDKTDAYNQKVY
GLSEAVVSVGYEYESCLDLTLWEKRTAILQGYELDASNMGGWTLDKHHVLDVQNGILYKGNGENQFIS
QQPPVVSSLEG

| | | |
|---|---|---|
| NOV40s, CG55069-02 | SEQ ID NO: 521 | 8645 bp |
| DNA Sequence | ORF Start: ATG at 151 | ORF Stop: TAA at 8314 |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAAGGCTTACCGCCTTCCCATACTCAGTATGACTTCGTGGAGCTCCT
GGATGGCAGCAGGCTGATTGCCAGAGAGCAGCGGAGCCTGCTTGAGACGGAGAGAGCCGGGCGGCAGG
CGAGATCCGTCAGCCTTCATGAGGCCGGCTTTATCCAGTACTTGGATTCTGGAATCTGGCATCTGGCT
TTTTATAATGATGGGAAAAATGCAGAGCAGGTGTCTTTTAATACCATTGTTATAGAGTCTGTGGTGGA
ATGTCCCCGAAATTGCCATGGAAATGGAGAATGCGTTTCTGGAACTTGCCATTGTTTTCCAGGATTTC
TGGGTCCGGATTGTTCAAGAGCCGCCTGTCCAGTGTTATGTAGTGGCAACGGGCAGTACTCCAAGGGC
CGCTGCCTGTGTTTCAGCGGCTGGAAGGGCACCGAGTGTGATGTGCCGACTACCCAGTGTATTGACCC
ACAGTGTGGGGGTCGTGGGATTTGTATCATGGGCTCCTGTGCTTGCAGCTCAGGATACAAAGGAGAAA
GTTGTGAAGAAGCTGACTGTATAGACCCTGGGTGTTCTAATCATGGTGTGTGTATCCACGGGGAATGT
CACTGCAGTCCAGGATGGGGAGGTAGCAATTGTGAAATACTGAAGACCATGTGTCCAGACCAGTGCTC
CGGCCACGGAACGTATCTTCAAGAAAGTGGCTCCTGCACGTGTGACCCTAACTGGACTGGCCCAGACT
GCTCAAACGAAATATGTTCTGTGGACTGTGGCTCACACGGCGTTTGCATGGGGGGGACGTGTCGCTGT
GAAGAAGGCTGGACGGGCCCAGCCTGTAATCAGAGAGCCTGCCACCCCCGCTGTGCCGAGCACGGGAC
CTGCAAGGATGGCAAGTGTGAATGCAGCCAGGGCTGGAATGGAGAGCACTGCACTATCGCTCACTATT
TGGATAAGATAGTTAAAGACAAGATAGGATATAAAGAGGGTTGTCCTGGTCTGTGCAACAGCAATGGA
AGATGTACCCTGGACCAAAATGGCGGACATTGTGTGTGCCAGCCTGGATGGAGAGGAGCAGGCTGTGA
CGTAGCCATGGAGACTCTTTGCACAGATAGCAAGGACAATGAAGGGGATGGACTCATTGACTGCATGG
ATCCCGATTGCTGCCTACAGAGTTCCTGCCAGAATCAGCCCTATTGTCGGGGACTGCCGGATCCTCAG
GACATCATTAGCCAAAGCCTTCAATCGCCTTCTCAGCAAGCTGCCAAATCCTTTTATGATCGAATCAG
TTTCCTTATAGGATCTGATAGCACCCATGTTATACCTGGAGAAAGTCCTTTCAATAAGAGCCTTGCAT
CTGTCATCAGAGGCCAAGTACTGACTGCTGATGGAACTCCACTTATTGGAGTAAATGTCTCGTTTTTC
CATTACCCAGAATATGGATATACTATTACCCGCCAGGACGGAATGTTTGACTTGGTGGCAAATGGTGG
GGCCTCTCTAACTTTGGTATTTGAACGATCCCCATTCCTCACTCAGTATCATACTGTGTGGATTCCAT
GGAATGTCTTTTATGTGATGGATACCCTAGTCATGGAGAAAGAAGAGAATGACATTCCCAGCTGTGAT
CTGAGTGGATTCGTGAGGCCAAATCCCATCATTGTGTCATCACCTTTATCCACCTTTTTCAGATCTTC
TCCTGAAGACAGTCCCATCATTCCCGAAACACAGGTACTCCACGAGGAAACTACAATTCCAGGAACAG
ATTTGAAACTCTCCTACTTGAGTTCCAGAGCTGCAGGGTATAAGTCAGTTCTCAAGATCACCATGACC
CAGTCTATTATTCCATTTAATTTAATGAAGGTTCATCTTATGGTAGCTGTAGTAGGAAGACTCTTCCA
AAAGTGGTTTCCTGCCTCACCAAACTTGGCCTATACTTTCATATGGGATAAAACAGATGCATATAATC
AGAAAGTCTATGGTCTATCTGAAGCTGTTGTGTCAGTTGGATATGAGTATGAGTCGTGTTTGGACCTG
ACTCTGTGGGAAAAGAGGACTGCCATTCTGCAGGGCTATGAATTGGATGCGTCCAACATGGGTGGCTG
GACATTAGATAAACATCACGTGCTGGATGTACAGAACGGTATACTGTACAAGGGAAACGGGGAAAACC
AGTTCATCTCCCAGCAGCCTCCAGTCGTGAGTAGCATCATGGGCAATGGGCGAAGGCGCAGCATTTCC
TGCCCCAGTTGCAATGGTCAAGCTGATGGTAACAAGTTACTGGCCCCAGTGGCGCTAGCTTGTGGGAT
CGATGGCAGTCTGTACGTAGGCGATTTCAACTACGTGCGGCGGATATTCCCTTCTGGAAATGTAACAA
GTGTCTTAGAACTAAGAAATAAAGATTTTAGACATAGCAGCAACCCAGCTCATAGATACTACCTTGCA
ACGGATCCAGTCACGGGAGATCTGTACGTTTCTGACACAAACACCCGCAGAATTTATCGCCCAAAGTC
ACTTACGGGGGCAAAAGACTTGACTAAAAATGCAGAAGTCGTCGCAGGGACAGGGGAGCAATGCCTTC
CGTTTGACGAGGCGAGATGTGGGGATGGAGGGAAGGCCGTGGAAGCCACACTCATGAGTCCCAAAGGA
ATGGCAGTTGATAAGAATGGATTAATCTACTTTGTTGATGGAACCATGATTAGGAAAGTTGACCAAAA
TGGAATCATATCAACTCTTCTGGGCTCTAACGATTTGACTTCAGCCAGACCTTTAACTTGTGACACCA
GCATGCACATCAGCCAGGTACGTCTGGAATGGCCCACTGACCTAGCCATTAACCCTATGGATAACTCC
ATTTATGTCCTGGATAATAATGTAGTTTTACAGATCACTGAAAATCGTCAAGTTCGCATTGCTGCTGG
ACGGCCCATGCACTGTCAGGTTCCCGGAGTGGAATATCCTGTGGGGAAGCACGCGGTGCAGACAACAC
TGGAATCAGCCACTGCCATTGCTGTGTCCTACAGTGGGGTCCTGTACATTACTGAAACTGATGAGAAG
AAAATTAACCGGATAAGGCAGGTCACAACAGATGGAGAAATCTCCTTAGTGGCCGGAATACCTTCAGA
GTGTGACTGCAAAAATGATGCCAACTGTGACTGTTACCAGAGTGGAGATGGCTACGCCAAGGATGCCA
AACTCAGTGCCCCATCCTCCCTGGCTGCTTCTCCAGATGGTACACTGTATATTGCAGATCTAGGGAAT
ATCCGGATCCGGGCTGTGTCAAAGAATAAGCCTTTACTTAACTCTATGAACTTCTATGAAGTTGCGTC
TCCAACTGATCAAGAACTCTACATCTTTGACATCAATGGTACTCACCAATATACTGTAAGTTTAGTCA
CTGGTGATTACCTTTACAATTTTAGCTACAGCAATGACAATGATATTACTGCTGTGACAGACAGCAAT
GGCAACACCCTTAGAATTAGACGGGACCCAAATCGCATGCCAGTTCGAGTGGTGTCTCCTGATAACCA
AGTGATATGGTTGACAATAGGAACAAATGGATGTTTGAAAGGCATGACTGCTCAAGGACTGGAATTAG
TTTTGTTTACTTACCATGGCAATAGTGGCCTTTTAGCCACTAAAAGTGATGAAACTGGATGGACAACG
TTTTTTGACTATGACAGTGAAGGTCGTCTGACAAATGTTACGTTTCCAACTGGAGTGGTCACAAACCT
GCATGGGGACATGGACAAGGCTATCACAGTGGACATTGAGTCATCTAGCCGAGAAGAAGATGTCAGCA
TCACTTCAAATCTGTCCTCGATCGATTCTTTCTACACCATGGTTCAAGATCAGTTAAGAAACAGCTAC
CAGATTGGTTATGACGGCTCCCTCAGAATTATCTACGCCAGTGGCCTGGACTCACACTACCAAACAGA
GCCGCACGTTCTGGCTGGCACCGCTAATCCGACGGTTGCCAAAAGAAACATGACTTTGCCTGGCGAGA
ACGGTCAAAACTTGGTGGAATGGAGATTCCGAAAAGAGCAAGCCCAAGGGAAAGTCAATGTCTTTGGC
CGCAAGCTCAGGGTTAATGGCAGAAACCTCCTTTCAGTTGACTTTGATCGAACAACAAAGACAGAAAA
GATCTATGACGACCACCGTAAATTTCTACTGAGGATCGCCTACGACACGTCTGGGCACCCGACTCTCT
GGCTGCCAAGCAGCAAGCTGATGGCCGTCAATGTCACCTATTCATCCACAGGTCAAATTGCCAGCATC
CAGCGAGGCACCACTAGCGAGAAAGTAGATTATGACGGACAGGGGAGGATCGTGTCTCGGGTCTTTGC
TGATGGTAAAACATGGAGTTACACATATTTAGAAAAGTCCATGGTTCTTCTGCTTCATAGCCAGCGGC
AGTACATCTTCGAATACGATATGTGGGACCGCCTGTCTGCCATCACCATGCCCAGTGTGGCTCGCCAC
ACCATGCAGACCATCCGATCCATTGGCTACTACCGCAACATATACAACCCCCCGGAAAGCAACGCCTC
CATCATCACGGACTACAACGAGGAAGGGCTGCTTCTACAAACAGCTTTCTTGGGTACAAGTCGGAGGG
TCTTATTCAAATACAGAAGGCAGACTAGGCTCTCAGAAATTTTATATGATAGCACAAGAGTCAGTTTT
ACCTATGATGAAACAGCAGGAGTCCTAAAGACAGTAAACCTCCAGAGTGATGGTTTTATTTGCACCAT
TAGATACAGGCAAATTGGTCCCCTGATTGACAGGCAGATTTTCCGCTTTAGTGAAGATGGGATGGTAA
ATGCAAGATTTGACTATAGCTATGACAACAGCTTTCGAGTGACCAGCATGCAGGGTGTGATCAATGAA
ACGCCACTGCCTATTGATCTGTATCAGTTTGATGACATTTCTGGCAAAGTTGAGCAGTTTGGAAAGTT
TGGAGTTATATATTATGATATTAACCAGATCATTTCTACAGCTGTAATGACCTATACGAAGCACTTTG
ATGCTCATGGCCGTATCAAGGAGATTCAATATGAGATATTCAGGTCGCTCATGTACTGGATTACAATT
CAGTATGATAACATGGGTCGGGTAACCAAGAGAGAGATTAAAATAGGGCCCTTTGCCAACACCACCAA
ATATGCTTATGAATATGATGTTGATGGACAGCTCCAAACAGTTTACCTCAATGAAAAGATAATGTGGC
GGTACAACTACGATCTGAATGGAAACCTCCATTTACTGAACCCAAGTAACAGTGCGCGTCTGACACCC
CTTCGCTATGACCTGCGAGACAGAATCACTCGACTGGGTGATGTTCAATATCGGTTGGATGAAGATGG
TTTCCTACGTCAAAGGGGCACGGAAATCTTTGAATATAGCTCCAAGGGGCTTCTAACTCGAGTTTACA
GTAAAGGCAGTGGCTGGACAGTGATCTACCGTTATGACGGCCTGGGAAGGCGTGTTTCTAGCAAAACC
AGTCTAGGACAGCACCTGCAGTTTTTTTATGCTGACTTAACTTATCCCACTAGGATTACTCATGTCTA
CAACCATTCGAGTTCAGAAATTACCTCCCTGTATTATGATCTCCAAGGACATCTTTTTGCCATGGAAA
TCAGCAGTGGGGATGAATTCTATATTGCATCGGATAACACAGGGACACCACTGGCTGTGTTCAGTAGC
AATGGGCTTATGCTGAAACAGATTCAGTACACTGCATATGGGGAAATCTATTTTGACTCTAATATTGA
CTTTCAACTGGTAATTGGATTTCATGGTGGCCTGTATGACCCACTCACCAAATTAATCCACTTTGGAG
AAAGAGATTATGACATTTTGGCAGGACGGTGGACAACACCTGACATAGAAATCTGGAAAAGAATTGGG
AAGGACCCAGCTCCTTTTAACTTGTACATGTTTAGGAATAACAACCCTGCAAGCAAAATCCATGACGT
GAAAGATTACATCACAGATGTTAACAGCTGGCTGGTGACATTTGGTTTCCATCTGCACAATGCTATTC
CTGGATTCCCTGTTCCCAAATTTGATTTAACAGAACCTTCTTACGAACTTGTGAAGAGTCAGCAGTGG
GATGATATACCGCCCATCTTCGGAGTCCAGCAGCAAGTGGCGCGGCAGGCCAAGGCCTTCCTGTCGCT
GGGGAAGATGGCCGAGGTGCAGGTGAGCCGGCGCCGGGCCGGCGGCGCGCAGTCCTGGCTGTGGTTCG
CCACGGTCAAGTCGCTGATCGGCAAGGGCGTCATGCTGGCCGTCAGCCAGGGCCGCGTGCAGACCAAC
GTGCTCAACATCGCCAACGAGGACTGCATCAAGGTGGCGGCCGTGCTCAACAACGCCTTCTACCTGGA
GAACCTGCACTTCACCATCGAGGGCAAGGACACGCACTACTTCATCAAGACCACCACGCCCGAGAGCG
ACCTGGGCACGCTGCGGTTGACCAGCGGCCGCAAGGCGCTGGAGAACGGCATCAACGTGACGGTGTCG
CAGTCCACCACGGTGGTGAACGGCAGGACGCGCAGGTTCGCGGACGTGGAGATGCAGTTCGGCGCGCT
GGCGCTGCACGTGCGCTACGGCATGACCCTGGACGAGGAGAAGGCGCGCATCCTGGAGCAGGCGCGGC
AGCGCGCGCTCGCCCGGGCCTGGGCGCGCGAGCAGCAGCGCGTGCGCGACGGCGAGGAGGGCGCGCGC
CTCTGGACGGAGGGCGAGAAGCGGCAGCTGCTGAGCGCCGGCAAGGTGCAGGGCTACGACGGGTACTA
CGTACTCTCGGTGGAGCAGTACCCCGAGCTGGCCGACAGCGCCAACAACATCCAGTTCCTGCGGCAGA
GCGAGATCGGCAGGAGGTAACGCCCGGGCCGCGCCCGCCGAGCCGCTCACGCCCTGCCCACATTGTCC
TGTGGCACAACCCGAGTGGGACTCTCCAACGCCCAAGAGCCTTCCTCCCGGGGGAATGAGACTGCTGT
TACGACCCACACCCACACCGCGAAAACAAGGACCGCTTTTTTCCGAATGACCTTAAAGGTGATCGGCT
TTAACGAATATGTTTACATATGCATAGCGCTGCACTCAGTCGGACTGAACGTAGCCAGAGGAAAAAAA
AATCATCAAGGACAAAGGCCTCGACCTGTTGCGCTGGGCCGTCTGTTCCTTCTAGGCACTGTATTTAA
CTAACTTTA

| | | | |
|---|---|---|---|
| NOV40s, CG55069-02 | SEQ ID NO: 522 | 2721 aa | MW at 303489.1kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHNPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLNWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKGLPPSHTQYDFVELLDGSRLIAREQRSLLETERAGRQARSVS
LHEAGFIQYLDSGIWHLAFYNDGKNAEQVSFNTIVIESVVECPRNCHGNGECVSGTCHCFPGFLGPDC
SRAACPVLCSGNGQYSKGRCLCFSGWKGTECDVPTTQCIDPQCGGRGICIMGSCACSSGYKGESCEEA
DCIDPGCSNHGVCIHGECHCSPGWGGSNCEILKTMCPDQCSGHGTYLQESGSCTCDPNWTGPDCSNEI
CSVDCGSHGVCMGGTCRCEEGWTGPACNQRACHPRCAEHGTCKDGKCECSQGWNGEHCTIAHYLDKIV
KDKIGYKEGCPGLCNSNGRCTLDQNGGHCVCQPGWRGAGCDVAMETLCTDSKDNEGDGLIDCMDPDCC
LQSSCQNQPYCRGLPDPQDIISQSLQSPSQQAAKSFYDRISFLIGSDSTHVIPGESPFNKSLASVIRG
QVLTADGTPLIGVNVSFFHYPEYGYTITRQDGMFDLVANGGASLTLVFERSPFLTQYHTVWIPWNVFY
VMDTLVMEKEENDIPSCDLSGFVRPNPIIVSSPLSTFFRSSPEDSPIIPETQVLHEETTIPGTDLKLS
YLSSRAAGYKSVLKITMTQSIIPFNLMKVHLMVAVVGRLFQKWFPASPNLAYTFIWDKTDAYNQKVYG
LSEAVVSVGYEYESCLDLTLWEKRTAILQGYELDASNNGGWTLDKHHVLDVQNGILYKGNGENQFISQ
QPPVVSSIMGNGRRRSISCPSCNGQADGNKLLAPVALACGIDGSLYVGDFNYVRRIFPSGNVTSVLEL
RNKDFRHSSNPAHRYYLATDPVTGDLYVSDTNTRRIYRPKSLTGAKDLTKNAEVVAGTGEQCLPFDEA
RCGDGGKAVEATLMSPKGMAVDKNGLIYFVDGTMIRKVDQNGIISTLLGSNDLTSARPLTCDTSMHIS
QVRLEWPTDLAINPMDNSIYVLDNNVVLQITENRQVRIAAGRPMHCQVPGVEYPVGKHAVQTTLESAT
AIAVSYSGVLYITETDEKKINRIRQVTTDGEISLVAGIPSECDCKNDANCDCYQSGDGYAKDAKLSAP
SSLAASPDGTLYIADLGNIRIRAVSKNKPLLNSMNFYEVASPTDQELYIFDINGTHQYTVSLVTGDYL
YNFSYSNDNDITAVTDSNGNTLRIRRDPNRMPVRVVSPDNQVIWLTIGTNGCLKGMTAQGLELVLFTY
HGNSGLLATKSDETGWTTFFDYDSEGRLTNVTFPTGVVTNLHGDMDKAITVDIESSSREEDVSITSNL
SSIDSFYTMVQDQLRNSYQIGYDGSLRIIYASGLDSHYQTEPHVLAGTANPTVAKRNMTLPGENGQNL
VEWRFRKEQAQGKVNVFGRKLRVNGRNLLSVDFDRTTKTEKIYDDHRKFLLRIAYDTSGHPTLWLPSS
KLMAVNVTYSSTGQIASIQRGTTSEKVDYDGQGRIVSRVFADGKTWSYTYLEKSMVLLLHSQRQYIFE
YDMWDRLSAITMPSVARHTMQTIRSIGYYRNIYNPPESNASIITDYNEEGLLLQTAFLGTSRRVLFKY
RRQTRLSEILYDSTRVSFTYDETAGVLKTVNLQSDGFICTIRYRQIGPLIDRQIFRFSEDGMVNARFD
YSYDNSFRVTSMQGVINETPLPIDLYQFDDISGKVEQFGKFGVIYYDINQIISTAVMTYTKHFDAHGR
IKEIQYEIFRSLMYWITIQYDNMGRVTKREIKIGPFANTTKYAYEYDVDGQLQTVYLNEKIMWRYNYD
LNGNLHLLNPSNSARLTPLRYDLRDRITRLGDVQYRLDEDGFLRQRGTEIFEYSSKGLLTRVYSKGSG
WTVIYRYDGLGRRVSSKTSLGQHLQFFYADLTYPTRITHVYNHSSSEITSLYYDLQGHLFAMEISSGD
EFYIASDNTGTPLAVFSSNGLMLKQIQYTAYGEIYFDSNIDFQLVIGFHGGLYDPLTKLIHFGERDYD
ILAGRWTTPDIEIWKRIGKDPAPFNLYMFRNNNPASKIHDVKDYITDVNSWLVTFGFHLHNAIPGFPV
PKFDLTEPSYELVKSQQWDDIPPIFGVQQQVARQAKAFLSLGKMAEVQVSRRRAGGAQSWLWFATVKS
LIGKGVMLAVSQGRVQTNVLNIANEDCIKVAAVLNNAFYLENLHFTIEGKDTHYFIKTTTPESDLGTL
RLTSGRKALENGINVTVSQSTTVVNGRTRRFADVEMQFGALALHVRYGMTLDEEKARILEQARQRALA
RAWAREQQRVRDGEEGARLWTEGEKRQLLSAGKVQGYDGYYVLSVEQYPELADSANNIQFLRQSEIGR
R

| | | |
|---|---|---|
| NOV40t, CG55069-05 | SEQ ID NO: 523 | 810 bp |
| DNA Sequence | ORF Start: at 7 | ORF Stop: at 805 |

AAGCTTTGTCCCCGAAATTGCCATGGAAATGGAGAATGCGTTTCTGGAACTTGCCATTGTTTTCCAGG
ATTTCTGGGTCCGGATTGTTCAAGAGCCGCCTGTCCAGTGTTATGTAGTGGCAACGGGCAGTACTCCA
AGGGCCGCTGCCTGTGTTTCAGCGGCTGGAAGGGCACCGAGTGTGATGTGCCGACTACCCAGTGTATT
GACCCACAGTGTGGGGGTCGTGGGATTTGTATCATGGGCTCTTGTGCTTGCAACTCAGGATACAAAGG
AGAAAGTTGTGAAGAAGCTGACTGTATAGACCCTGGGTGTTCTAATCATGGTGTGTGTATCCACGGGG
AATGTCACTGCAGTCCAGGATGGGGAGGTAGCAATTGTGAAATACTGAAGACCATGTGTCCAGACCAG
TGCTCCGGCCACGGAACGTATCTTCAAGAAAGTGGCTCCTGCACGTGTGACCCTAACTGGACTGGCCC
AGACTGCTCAAACGAAATATGTTCTGTGGACTGTGGCTCACACGGCGTTTGCATGGGGGGGACGTGTC
GCTGTGAAGAAGGCTGGACGGGCCCAGCCTGTAATCAGAGAGCCTGCCACCCCCGCTGTGCCGAGCAC
GGGACCTGCAAGGATGGCAAGTGTGAATGCAGCCAGGGCTGGAATGGAGAGCACTGCACTATCGCTCA
CTATTTGGATAAGATAGTTAAAGAGGGTTGTCCTGGTCTGTGCAACAGCAATGGAAGATGTACCCTGG
ACCAAAATGGCTGGCATTGTGTGTGCCAGCCTGGATGGAGAGGAGCAGGCTGTGACGTCGAC

| | | | |
|---|---|---|---|
| NOV40t, GG55069-05 | SEQ ID NO: 524 | 266 aa | MW at 27935.0kD |
| Protein Sequence | | | |

CPRNCHGNGECVSGTCHCFPGFLGPDCSRAACPVLCSGNGQYSKGRCLCFSGWKGTECDVPTTQCIDP
QCGGRGICIMGSCACNSGYKGESCEEADCIDPGCSNHGVCIHGECHCSPGWGGSNCEILKTMCPDQCS
GHGTYLQESGSCTCDPNWTGPDCSNEICSVDCGSHGVCMGGTCRCEEGWTGPACNQRACHPRCAEHGT
CKDGKCECSQGWNGEHCTIAHYLDKIVKEGCPGLCNSNGRCTLDQNGWHCVCQPGWRGAGCD

| | | |
|---|---|---|
| NOV40u, CG55069-06 | SEQ ID NO: 525 | 8204 bp |
| DNA Sequence | ORF Start: ATG at 4 | ORF Stop: TAA at 8179 |

AGTATGGATGTGAAAGAACGCAGGCCTTACTGCTCCCTGACCAAGAGCAGACGAGAGAAGGAACGGCG
CTACACAAATTCCTCCGCAGACAATGAGGAGTGCCGGGTACCCACACAGAAGTCCTACAGTTCCAGCG
AGACATTGAAAGCTTTTGATCATGATTCCTCGCGGCTGCTTTACGGCAACAGAGTGAAGGATTTGGTT
CACAGAGAAGCAGACGAGTTCACTAGACAAGGACAGAATTTTACCCTAAGGCAGTTAGGAGTTTGTGA
ACCAGCAACTCGAAGAGGACTGGCATTTTGTGCGGAAATGGGGCTCCCTCACAGAGGTTACTCTATCA
GTGCAGGGTCAGATGCTGATACTGAAAATGAAGCAGTGATGTCCCCAGAGCATGCCATGAGACTTTGG
GGCAGGGGGGTCAAATCAGGCCGCAGCTCCTGCCTGTCAAGTCGGTCCAACTCAGCCCTCACCCTGAC
AGATACGGAGCACGAAAACAAGTCCGACAGTGAGAATGAGCAACCTGCAAGCAATCAAGGCCAGTCTA
CCCTGCAGCCCTTGCCGCCTTCCCATAAGCAGCACTCTGCACAGCATCATCCATCCATCACTTCTCTC
AACAGAAACTCCCTGACCAATAGAAGGAACCAGAGTCCGGCCCCGCCGGCTGCTTTGCCCGCCGAGCT
GCAAACCACACCCGAGTCCGTCCAGCTGCAGGACAGCTGGGTCCTTGGCAGTAATGTACCACTGGAAA
GCAGGCATTTCCTATTCAAAACAGGAACAGGTACAACGCCACTGTTCAGTACTGCAACCCCAGGATAC
ACAATGGCATCTGGCTCTGTTTATTCACCACCTACTCGGCCACTACCTAGAAACACCCTATCAAGAAG
TGCTTTTAAATTCAAGAAGTCTTCAAAGTACTGTAGCTGGAAATGCACTGCACTGTGTGCCGTAGGGG
TCTCGGTGCTCCTGGCAATACTCCTGTCTTATTTTATAGCAATGCATCTCTTTGGCCTCAACTGGCAG
CTACAGCAGACTGAAAATGACACATTTGAGAATGGAAAAGTGAATTCTGATACCATGCCAACAAACAC
TGTGTCATTACCTTCTGGAGACAATGGAAAATTAGGTGGATTTACGCAAGAAAATAACACCATAGATT
CCGGAGAACTTGATATTGGCCGAAGAGCAATTCAAGAGATTCCTCCCGGGATCTTCTGGAGATCACAG
CTCTTCATTGATCAGCCACAGTTTCTTAAATTCAATATCTCTCTTCAGAAGGATGCATTGATTGGAGT
ATATGGCCGGAAGAAGTTACCGCCTTCCCATACTCAGTCCTCCCCCCAGTATGACTTCGTGGAGCTCC
TGGATGGCAGCAGGCTGATTGCCAGAGAGCAGCGGAGCCTGCTTGAGACGGAGAGAGCCGGGCGGCAG
GCGAGATCCGTCAGCCTTCATGAGGCCGGCTTTATCCAGTACTTGGATTCTGGAATCTGGCATCTGGC
TTTTTATAATGATGGGAAAAATGCAGAGCAGGTGTCTTTTAATACCATTGTTATAGAGTCTGTGGTGG
AATGTCCCCGAAATTGCCATGGAAATGGAGAATGCGTTTCTGGAACTTGCCATTGTTTTCCAGGATTT
CTGGGTCCGGATTGTTCAAGAGCCGCCTGTCCAGTGTTATGTAGTGGCAACGGGCAGTACTCCAAGGG
CCGCTGCCTGTGTTTCAGCGGCTGGAAGGGCACCGAGTGTGATGTGCCGACTACCCAGTGTATTGACC
CACAGTGTGGGGGTCGTGGGATTTGTATCATGGGCTCCTGTGCTTGCAGCTCAGGATACAAAGGAGAA
AGTTGTGAAGAAGCTGACTGTATAGACCCTGGGTGTTCTAATCATGGTGTGTGTATCCACGGGGAATG
TCACTGCAGTCCAGGATGGGGAGGTAGCAATTGTGAAATACTGAAGACCATGTGTCCAGACCAGTGCT
CCGGCCACGGAACGTATCTTCAAGAAAGTGGCTCCTGCACGTGTGACCCTAACTGGACTGGCCCAGAC
TGCTCAAACGAAATATGTTCTGTGGACTGTGGCTCACACGGCGTTTGCATGGGGGGGACGTGTCGCTG
TGAAGAAGGCTGGACGGGCCCAGCCTGTAATCAGAGAGCCTGCCACCCCCGCTGTGCCGAGCACGGGA
CCTGCAAGGATGGCAAGTGTGAATGCAGCCAGGGCTGGAATGGAGAGCACTGCACTATCGCTCACTAT
TTGGATAAGATAGTTAAAGACAAGATAGGATATAAAGAGGGTTGTCCTGGTCTGTGCAACAGCAATGG
AAGATGTACCCTGGACCAAAATGGCGGACATTGTGTGTGCCAGCCTGGATGGAGAGGAGCAGGCTGTG
ACGTAGCCATGGAGACTCTTTGCACAGATAGCAAGGACAATGAAGGGGATGGACTCATTGACTGCATG
GATCCCGATTGCTGCCTACAGAGTTCCTGCCAGAATCAGCCCTATTGTCGGGGACTGCCGGATCCTCA
GGACATCATTAGCCAAAGCCTTCAATCGCCTTCTCAGCAAGCTGCCAAATCCTTTTATGATCGAATCA
GTTTCCTTATAGGATCTGATAGCACCCATGTTATACCTGGAGAAAGTCCTTTCAATAAGAGCCTTGCA
TCTGTCATCAGAGGCCAAGTACTGACTGCTGATGGAACTCCACTTATTGGAGTAAATGTCTCGTTTTT
CCATTACCCAGAATATGGATATACTATTACCCGCCAGGACGGAATGTTTGACTTGGTGGCAAATGGTG
GGGCCTCTCTAACTTTGGTATTTGAACGATCCCCATTCCTCACTCAGTATCATACTGTGTGGATTCCA
TGGAATGTCTTTTATGTGATGGATACCCTAGTCATGGAGAAAGAAGAGAATGACATTCCCAGCTGTGA
TCTGAGTGGATTCGTGAGGCCAAATCCCATCATTGTGTCATCACCTTTATCCACCTTTTTCAGATCTT
CTCCTGAAGACAGTCCCATCATTCCCGAAACACAGGTACTCCACGAGGAAACTACAATTCCAGGAACA
GATTTGAAACTCTCCTACTTGAGTTCCAGAGCTGCAGGGTATAAGTCAGTTCTCAAGATCACCATGAC
CCAGTCTATTATTCCATTTAATTTAATGAAGGTTCATCTTATGGTAGCTGTAGTAGGAAGACTCTTCC
AAAAGTGGTTTCCTGCCTCACCAAACTTGGCCTATACTTTCATATGGGATAAAACAGATGCATATAAT
CAGAAAGTCTATGGTCTATCTGAAGCTGTTGTGTCAGTTGGATATGAGTATGAGTCGTGTTTGGACCT
GACTCTGTGGGAAAAGAGGACTGCCATTCTGCAGGGCTATGAATTGGATGCGTCCAACATGGGTGGCT
GGACATTAGATAAACATCACGTGCTGGATGTACAGAACGGTATACTGTACAAGGGAAACGGGGAAAAC
CAGTTCATCTCCCAGCAGCCTCCAGTCGTGAGTAGCATCATGGGCAATGGGCGAAGGCGCAGCATTTC
CTGCCCCAGTTGCAATGGTCAAGCTGATGGTAACAAGTTACTGGCCCCAGTGGCGCTAGCTTGTGGGA
TCGATGGCAGTCTGTACGTAGGCGATTTCAACTACGTGCGGCGGATATTCCCTTCTGGAAATGTAACA
AGTGTCTTAGAACTAAGAAATAAAGATTTTAGACATAGCAGCAACCCAGCTCATAGATACTACCTTGC
AACGGATCCAGTCACGGGAGATCTGTACGTTTCTGACACAAACACCCGCAGAATTTATCGCCCAAAGT
CACTTACGGGGGCAAAAGACTTGACTAAAAATGCAGAAGTCGTCGCAGGGACAGGGGAGCAATGCCTT
CCGTTTGACGAGGCGAGATGTGGGGATGGAGGGAAGGCCGTGGAAGCCACACTCATGAGTCCCAAAGG
AATGGCAGTTGATAAGAATGGATTAATCTACTTTGTTGATGGAACCATGATTAGGAAAGTTGACCAAA
ATGGAATCATATCAACTCTTCTGGGCTCTAACGATTTGACTTCAGCCAGACCTTTAACTTGTGACACC
AGCATGCACATCAGCCAGGTACGTCTGGAATGGCCCACTGACCTAGCCATTAACCCTATGGATAACTC
CATTTATGTCCTGGATAATAATGTAGTTTTACAGATCACTGAAAATCGTCAAGTTCGCATTGCTGCTG
GACGGCCCATGCACTGTCAGGTTCCCGGAGTGGAATATCCTGTGGGGAAGCACGCGGTGCAGACAACA
CTGGAATCAGCCACTGCCATTGCTGTGTCCTACAGTGGGGTCCTGTACATTACTGAAACTGATGAGAA
GAAAATTAACCGGATAAGGCAGGTCACAACAGATGGAGAAATCTCCTTAGTGGCCGGAATACCTTCAG
AGTGTGACTGCAAAAATGATGCCAACTGTGACTGTTACCAGAGTGGAGATGGCTACGCCAAGGATGCC
AAACTCAGTGCCCCATCCTCCCTGGCTGCTTCTCCAGATGGTACACTGTATATTGCAGATCTAGGGAA
TATCCGGATCCGGGCTGTGTCAAAGAATAAGCCTTTACTTAACTCTATGAACTTCTATGAAGTTGCGT
CTCCAACTGATCAAGAACTCTACATCTTTGACATCAATGGTACTCACCAATATACTGTAAGTTTAGTC
ACTGGTGATTACCTTTACAATTTTAGCTACAGCAATGACAATGATATTACTGCTGTGACAGACAGCAA
TGGCAACACCCTTAGAATTAGACGGGACCCAAATCGCATGCCAGTTCGAGTGGTGTCTCCTGATAACC
AAGTGATATGGTTGACAATAGGAACAAATGGATGTTTGAAAGGCATGACTGCTCAAGGACTGGAATTA
GTTTTGTTTACTTACCATGGCAATAGTGGCCTTTTAGCCACTAAAAGTGATGAAACTGGATGGACAAC
GTTTTTTGACTATGACAGTGAAGGTCGTCTGACAAATGTTACGTTTCCAACTGGAGTGGTCACAAACC
TGCATGGGGACATGGACAAGGCTATCACAGTGGACATTGAGTCATCTAGCCGAGAAGAAGATGTCAGC
ATCACTTCAAATCTGTCCTCGATCGATTCTTTCTACACCATGGTTCAAGATCAGTTAAGAAACAGCTA
CCAGATTGGTTATGACGGCTCCCTCAGAATTATCTACGCCAGTGGCCTGGACTCACACTACCAAACAG
AGCCGCACGTTCTGGCTGGCACCGCTAATCCGACGGTTGCCAAAAGAAACATGACTTTGCCTGGCGAG
AACGGTCAAAACTTGGTGGAATGGAGATTCCGAAAAGAGCAAGCCCAAGGGAAAGTCAATGTCTTTGG
CCGCAAGCTCAGGGTTAATGGCAGAAACCTCCTTTCAGTTGACTTTGATCGAACAACAAAGACAGAAA
AGATCTATGACGACCACCGTAAATTTCTACTGAGGATCGCCTACGACACGTCTGGGCACCCGACTCTC
TGGCTGCCAAGCAGCAAGCTGATGGCCGTCAATGTCACCTATTCATCCACAGGTCAAATTGCCAGCAT
CCAGCGAGGCACCACTAGCGAGAAAGTAGATTATGACGGACAGGGGAGGATCGTGTCTCGGGTCTTTG
CTGATGGTAAAACATGGAGTTACACATATTTAGAAAAGTCCATGGTTCTTCTGCTTCATAGCCAGCGG
CAGTACATCTTCGAATACGATATGTGGGACCGCCTGTCTGCCATCACCATGCCCAGTGTGGCTCGCCA
CACCATGCAGACCATCCGATCCATTGGCTACTACCGCAACATATACAACCCCCCGGAAAGCAACGCCT
CCATCATCACGGACTACAACGAGGAAGGGCTGCTTCTACAAACAGCTTTCTTGGGTACAAGTCGGAGG
GTCTTATTCAAATACAGAAGGCAGACTAGGCTCTCAGAAATTTTATATGATAGCACAAGAGTCAGTTT
TACCTATGATGAAACAGCAGGAGTCCTAAAGACAGTAAACCTCCAGAGTGATGGTTTTATTTGCACCA
TTAGATACAGGCAAATTGGTCCCCTGATTGACAGGCAGATTTTCCGCTTTAGTGAAGATGGGATGGTA
AATGCAAGATTTGACTATAGCTATGACAACAGCTTTCGAGTGACCAGCATGCAGGGTGTGATCAATGA
AACGCCACTGCCTATTGATCTGTATCAGTTTGATGACATTTCTGGCAAAGTTGAGCAGTTTGGAAAGT
TTGGAGTTATATATTATGATATTAACCAGATCATTTCTACAGCTGTAATGACCTATACGAAGCACTTT
GATGCTCATGGCCGTATCAAGGAGATTCAATATGAGATATTCAGGTCGCTCATGTACTGGATTACAAT
TCAGTATGATAACATGGGTCGGGTAACCAAGAGAGAGATTAAAATAGGGCCCTTTGCCAACACCACCA
AATATGCTTATGAATATGATGTTGATGGACAGCTCCAAACAGTTTACCTCAATGAAAAGATAATGTGG
CGGTACAACTACGATCTGAATGGAAACCTCCATTTACTGAACCCAAGTAACAGTGCGCGTCTGACACC
CCTTCGCTATGACCTGCGAGACAGAATCACTCGACTGGGTGATGTTCAATATCGGTTGGATGAAGATG
GTTTCCTACGTCAAAGGGGCACGGAAATCTTTGAATATAGCTCCAAGGGGCTTCTAACTCGAGTTTAC
AGTAAAGGCAGTGGCTGGACAGTGATCTACCGTTATGACGGCCTGGGAAGGCGTGTTTCTAGCAAAAC
CAGTCTAGGACAGCACCTGCAGTTTTTTTATGCTGACTTAACTTATCCCACTAGGATTACTCATGTCT
ACAACCATTCGAGTTCAGAAATTACCTCCCTGTATTATGATCTCCAAGGACATCTTTTTGCCATGGAA
ATCAGCAGTGGGGATGAATTCTATATTGCATCGGATAACACAGGGACACCACTGGCTGTGTTCAGTAG
CAATGGGCTTATGCTGAAACAGATTCAGTACACTGCATATGGGGAAATCTATTTTGACTCTAATATTG
ACTTTCAACTGGTAATTGGATTTCATGGTGGCCTGTATGACCCACTCACCAAATTAATCCACTTTGGA
GAAAGAGATTATGACATTTTGGCAGGACGGTGGACAACACCTGACATAGAAATCTGGAAAAGAATTGG
GAAGGACCCAGCTCCTTTTAACTTGTACATGTTTAGGAATAACAACCCTGCAAGCAAAATCCATGACG
TGAAAGATTACATCACAGATGTTAACAGCTGGCTGGTGACATTTGGTTTCCATCTGCACAATGCTATT
CCTGGATTCCCTGTTCCCAAATTTGATTTAACAGAACCTTCTTACGAACTTGTGAAGAGTCAGCAGTG
GGATGATATACCGCCCATCTTCGGAGTCCAGCAGCAAGTGGCGCGGCAGGCCAAGGCCTTCCTGTCGC
TGGGGAAGATGGCCGAGGTGCAGGTGAGCCGGCGCCGGGCCGGCGGCGCGCAGTCCTGGCTGTGGTTC
GCCACGGTCAAGTCGCTGATCGGCAAGGGCGTCATGCTGGCCGTCAGCCAGGGCCGCGTGCAGACCAA
CGTGCTCAACATCGCCAACGAGGACTGCATCAAGGTGGCGGCCGTGCTCAACAACGCCTTCTACCTGG
AGAACCTGCACTTCACCATCGAGGGCAAGGACACGCACTACTTCATCAAGACCACCACGCCCGAGAGC
GACCTGGGCACGCTGCGGTTGACCAGCGGCCGCAAGGCGCTGGAGAACGGCATCAACGTGACGGTGTC
GCAGTCCACCACGGTGGTGAACGGCAGGACGCGCAGGTTCGCGGACGTGGAGATGCAGTTCGGCGCGC
TGGCGCTGCACGTGCGCTACGGCATGACCCTGGACGAGGAGAAGGCGCGCATCCTGGAGCAGGCGCGG
CAGCGCGCGCTCGCCCGGGCCTGGGCGCGCGAGCAGCAGCGCGTGCGCGACGGCGAGGAGGGCGCGCG
CCTCTGGACGGAGGGCGAGAAGCGGCAGCTGCTGAGCGCCGGCAAGGTGCAGGGCTACGACGGGTACT
ACGTACTCTCGGTGGAGCAGTACCCCGAGCTGGCCGACAGCGCCAACAACATCCAGTTCCTGCGGCAG
AGCGAGATCGGCAGGAGGTAACGCCCGGGCCGCGCCCGCCGAGC

| | | | |
|---|---|---|---|
| NOV40u, CG55069-06 | SEQ ID NO: 526 | 2725 aa | MW at 303959.6kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNOGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIANHLFGLNWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFThDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNQPYCRGLPDPQDIISOSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRNPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNNTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTNQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIOYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGALALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40v, CG55069-07 | SEQ ID NO: 527 | 1833 bp |
| DNA Sequence | ORF Start: at 7 | ORF Stop: at 1828 |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATTTGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTTTTTCAGATCTTCTCCTGA
AGACAGTCCCATCATTCCCGAAACACAGGTACTCCACGAGGAAACTACAATTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTTTAACTTGTGACACCAGCATGCACATCAGCCAGGTACGTCTGG
AATGGCCCACTGACCTAGCCATTAACCCTATGGATAACTCCATTTATGTCCTGGATAATGTCGAC

| | | | |
|---|---|---|---|
| NOV40v, GG55069-07 | SEQ ID NO: 528 | 607 aa | MW at 66606.6kD |
| Protein Sequence | | | |

DQNGGHCVCQPGWRGAGCDVAMETLCTDSKDNEGDGLIDCMDPDCCLQSSCQNQPYCRGLPDPQDIIS
QSLQSPSQQAAKSFYDRISFLIGSDSTHVIPGESPFNKSLASVIRGOVLTADGTPLIGVNVSFFHYPE
YGYTITRQDGMFDLVANGGASLTLVFERSPFLTQYHTVWIPWNVFYVMDTLVMKKEENDIPSCDLSGF
VRPNPIIVSSPLSTFFRSSPEDSPIIPETQVLHEETTIPGTDLKLSYLSSRAAGYKSVLKITMTQSII
PFNLMKVHLMVAVVGRLFQKWFPASPNLAYTFIWDKTDAYNQKVYGLSEAVVSVGYEYESCLDLTLWE
KRTAILQGYELDASNMGGWTLDKHHVLDVQNGILYKGNGENQFISQQPPVVSSIMGNGRRRSISCPSC
NGQADGNKLLAPVALACGIDGSLYVGDFNYVRRIFPSGNVTSVLELSSNPAHRYYLATDPVTGDLYVS
DTNTRRIYRPKSLTGAKDLTKNAEVVAGTGEQCLPFDEARCGDGGKAVEATLMSPKGMAVDKNGLIYF
VDGTMIRKVDQNGIISTLLGSNDLTSARPLTCDTSMHISQVRLEWPTDLAINPMDNSIYVLDN

| | | |
|---|---|---|
| NOV40w, CG55069-08 | SEQ ID NO: 529 | 8487 bp |
| DNA Sequence | ORF Start: ATG at 299 | ORF Stop: TAA at 8138 |

ACTCACTATAGGGCTCGAGCGGCCGCCCGGGCAGGTCCCATTTGACAGAAAAAGGCAGTAAACGGGGA
ATCTCTTTTTTTGAATAAAGAAGAAGAAGAAATAAAGTACCTGTCATCTTGACAAGTGGCGGAGCGGA
GGAGTCAAGGATTATAAATGATCACAGCCAGGTCCAGCTCGCCCCGTGATTGGGCTCTCCCGCGATCT
GCACCGGGGGAAGCGCATGAGAGGCCAATGAGACTTGAACCCTGAGCCTAAGTTGTCACCAGCAGGAC
TGATGTGCACACAGAAGGAATGAAGTATGGATGTGAAAGAACGCAGGCCTTACTGCTCCCTGACCAAG
AGCAGACGAGAGAAGGAACGGCGCTACACAAATTCCTCCGCAGACAATGAGGAGTGCCGGGTACCCAC
ACAGAAGTCCTACAGTTCCAGCGAGACATTGAAAGCTTTTGATCATGATTCCTCGCGGCTGCTTTACG
GCAACAGAGTGAAGGATTTGGTTCACAGAGAAGCAGACGAGTTCACTAGACAAGAGCAACCTGCAAGC
AATCAAGGCCAGTCTACCCTGCAGCCCTTGCCGCCTTCCCATAAGCAGCACTCTGCACAGCATCATCC
ATCCATCACTTCTCTCAACAGAAACTCCCTGACCAATAGAAGGAACCAGAGTCCGGCCCCGCCGGCTG
CTTTGCCCGCCGAGCTGCAAACCACACCCGAGTCCGTCCAGCTGCAGGACAGCTGGGTCCTTGGCAGT
AATGTACCACTGGAAAGCAGGCATTTCCTATTCAAAACAGGAACAGGTACAACGCCACTGTTCAGTAC
TGCAACCCCAGGATACACAATGGCATCTGGCTCTGTTTATTCACCACCTACTCGGCCACTACCTAGAA
ACACCCTATCAAGAAGTGCTTTTAAATTCAAGAAGTCTTCAAAGTACTGTAGCTGGAAATGCACTGCA
CTGTGTGCCGTAGGGGTCTCGGTGCTCCTGGCAATACTCCTGTCTTATTTTATAGCAATGCATCTCTT
TGGCCTCAACTGGCAGCTACAGCAGACTGAAAATGACACATTTGAGAATGGAAAAGTGAATTCTGATA
CCATGCCAACAAACACTGTGTCATTACCTTCTGGAGACAATGGAAAATTAGGTGGATTTACGCAAGAA
AATAACACCATAGATTCCGGAGAACTTGATATTGGCCGAAGAGCAATTCAAGAGATTCCTCCCGGGAT
CTTCTGGAGATCACAGCTCTTCATTGATCAGCCACAGTTTCTTAAATTCAATATCTCTCTTCAGAAGG
ATGCATTGATTGGAGTATATGGCCGGAAAGGCTTACCGCCTTCCCATACTCAGTATGACTTCGTGGAG
CTCCTGGATGGCAGCAGGCTGATTGCCAGAGAGCAGCGGAGCCTGCTTGAGACGGAGAGAGCCGGGCG
GCAGGCGAGATCCGTCAGCCTTCATGAGGCCGGCTTTATCCAGTACTTGGATTCTGGAATCTGGCATC
TGGCTTTTTATAATGATGGGAAAAATGCAGAGCAGGTGTCTTTTAATACCATTGTTATAGAGTCTGTG
GTGGAATGTCCCCGAAATTGCCATGGAAATGGAGAATGCGTTTCTGGAACTTGCCATTGTTTTCCAGG
ATTTCTGGGTCCGGATTGTTCAAGAGCCGCCTGTCCAGTGTTATGTAGTGGCAACGGGCAGTACTCCA
AGGGCCGCTGCCTGTGTTTCAGCGGCTGGAAGGGCACCGAGTGTGATGTGCCGACTACCCAGTGTATT
GACCCACAGTGTGGGGGTCGTGGGATTTGTATCATGGGCTCCTGTGCTTGCAACTCAGGATACAAAGG
AGAAAGTTGTGAAGAAGCTGACTGTATAGACCCTGGGTGTTCTAATCATGGTGTGTGTATCCACGGGG
AATGTCACTGCAGTCCAGGATGGGGAGGTAGCAATTGTGAAATACTGAAGACCATGTGTCCAGACCAG
TGCTCCGGCCACGGAACGTATCTTCAAGAAAGTGGCTCCTGCACGTGTGACCCTAACTGGACTGGCCC
AGACTGCTCAAACGAAATATGTTCTGTGGACTGTGGCTCACACGGCGTTTGCATGGGGGGGACGTGTC
GCTGTGAAGAAGGCTGGACGGGCCCAACCTGTAATCAGAGAGCCTGCCACCCCCGCTGTGCCGAGCAC
GGGACCTGCAAGGATGGCAAGTGTGAATGCAGCCATGGCTGGAATGGAGAGCACTGCACTATCGAGGG
TTGTCCTGGTCTGTGCAACAGCAATGGAAGATGTACCCTGGACCAAAATGGCTGGCATTGTGTGTGCC
AGCCTGGATGGAGAGGAGCAGGCTGTGACGTAGCCATGGAGACTCTTTGCACAGATAGCAAGGACAAT
GAAGGAGATGGACTCATTGACTGCATGGATCCCGATTGCTGCCTACAGAGTTCCTGCCAGAATCAGCC
CTATTGTCGGGGACTGCCGGATCCTCAGGACATCATTAGCCAAAGCCTTCAATCGCCTTCTCAGCAAG
CTGCCAAATCCTTTTATGATCGAATCAGTTTCCTTATAGGATCTGATAGCACCCATGTTATACCTGGA
GAAAGTCCTTTCAATAAGAGCCTTGCATCTGTCATCAGAGGCCAAGTACTGACTGCTGATGGAACTCC
ACTTATTGGAGTAAATGTCTCGTTTTTCCATTACCCAGAATATGGATATACTATTACCCGCCAGGACG
GAATGTTTGACTTGGTGGCAAATGGTGGGGCCTCTCTAACTTTGGTATTTGAACGATCCCCATTCCTC
ACTCAGTATCATACTGTGTGGATTCCATGGAATGTCTTTTATGTGATGGATACCCTAGTCATGGAGAA
AGAAGAGAATGACATTCCCAGCTGTGATCTGAGTGGATTCGTGAGGCCAAATCCCATCATTGTGTCAT
CACCTTTATCCACCTTTTTCAGATCTTCTCCTGAAGACAGTCCCATCATTCCCGAAACACAGGTACTC
CACGAGGAAACTACAATTCCAGGAACAGATTTGAAACTCTCCTACTTGAGTTCCAGAGCTGCAGGGTA
TAAGTCAGTTCTCAAGATCACCATGACCCAGTCTATTATTCCATTTAATTTAATGAAGGTTCATCTTA
TGGTAGCTGTAGTAGGAAGACTCTTCCAAAAGTGGTTTCCTGCCTCACCAAACTTGGCCTATACTTTC
ATATGGGATAAAACAGATGCATATAATCAGAAAGTCTATGGTCTATCTGAAGCTGTTGTGTCAGTTGG
ATATGAGTATGAGTCGTGTTTGGACCTGACTCTGTGGGAAAAGAGGACTGCCATTCTGCAGGGCTATG
AATTGGATGCGTCCAACATGGGTGGCTGGACATTAGATAAACATCACGTGCTGGATGTACAGAACGGT
ATACTGTACAAGGGAAACGGGGAAAACCAGTTCATCTCCCAGCAGCCTCCAGTCGTGAGTAGCATCAT
GGGCAATGGGCGAAGGCGCAGCATTTCCTGCCCCAGTTGCAATGGTCAAGCTGATGGTAACAAGTTAC
TGGCCCCAGTGGCGCTAGCTTGTGGGATCGATGGCAGTCTGTACGTAGGCGATTTCAACTACGTGCGG
CGGATATTCCCTTCTGGAAATGTAACAAGTGTCTTAGAACTAAGAAATAAAGATTTTAGACATAGCAG
CAACCCAGCTCATAGATACTACCTTGCAACGGATCCAGTCACGGGAGATCTGTACGTTTCTGACACAA
ACACCCGCAGAATTTATCGCCCAAAGTCACTTACGGGGGCAAAAGACTTGACTAAAAATGCAGAAGTC
GTCGCAGGGACAGGGGAGCAATGCCTTCCGTTTGACGAGGCGAGATGTGGGGATGGAGGGAAGGCCGT
GGAAGCCACACTCATGAGTCCCAAAGGAATGGCAGTTGATAAGAATGGATTAATCTACTTTGTTGATG
GAACCATGATTAGGAAAGTTGACCAAAATGGAATCATATCAACTCTTCTGGGCTCTAACGATTTGACT
TCAGCCAGACCTTTAACTTGTGACACCAGCATGCACATCAGCCAGGTACGTCTGGAATGGCCCACTGA
CCTAGCCATTAACCCTATGGATAACTCCATTTATGTCCTGGATAATAATGTAGTTTTACAGATCACTG
AAAATCGTCAAGTTCGCATTGCTGCTGGACGGCCCATGCACTGTCAGGTTCCCGGAGTGGAATATCCT
GTGGGGAAGCACGCGGTGCAGACAACACTGGAATCAGCCACTGCCATTGCTGTGTCCTACAGTGGGGT
CCTGTACATTACTGAAACTGATGAGAAGAAAATTAACCGGATAAGGCAGGTCACAACAGATGGAGAAA
TCTCCTTAGTGGCCGGAATACCTTCAGAGTGTGACTGCAAAAATGATGCCAACTGTGACTGTTACCAG
AGTGGAGATGGCTACGCCAAGGATGCCAAACTCAGTGCCCCATCCTCCCTGGCTGCTTCTCCAGATGG
TACACTGTATATTGCAGATCTAGGGAATATCCGGATCCGGGCTGTGTCAAAGAATAAGCCTTTACTTA
ACTCTATGAACTTCTATGAAGTTGCGTCTCCAACTGATCAAGAACTCTACATCTTTGACATCAATGGT
ACTCACCAATATACTGTAAGTTTAGTCACTGGTGATTACCTTTACAATTTTAGCTACAGCAATGACAA
TGATATTACTGCTGTGACAGACAGCAATGGCAACACCCTTAGAATTAGACGGGACCCAAATCGCATGC
CAGTTCGAGTGGTGTCTCCTGATAACCAAGTGATATGGTTGACAATAGGAACAAATGGATGTTTGAAA
GGCATGACTGCTCAAGGACTGGAATTAGTTTTGTTTACTTACCATGGCAATAGTGGCCTTTTAGCCAC
TAAAAGTGATGAAACTGGATGGACAACGTTTTTTGACTATGACAGTGAAGGTCGTCTGACAAATGTTA
CGTTTCCAACTGGAGTGGTCACAAACCTGCATGGGGACATGGACAAGGCTATCACAGTGGACATTGAG
TCATCTAGCCGAGAAGAAGATGTCAGCATCACTTCAAATCTGTCCTCGATCGATTCTTTCTACACCAT
GGTTCAAGATCAGTTAAGAAACAGCTACCAGATTGGTTATGACGGCTCCCTCAGAATTATCTACGCCA
GTGGCCTGGACTCACACTACCAAACAGAGCCGCACGTTCTGGCTGGCACCGCTAATCCGACGGTTGCC
AAAAGAAACATGACTTTGCCTGGCGAGAACGGTCAAAACTTGGTGGAATGGAGATTCCGAAAAGAGCA
AGCCCAAGGGAAAGTCAATGTCTTTGGCCGCAAGCTCAGGGTTAATGGCAGAAACCTCCTTTCAGTTG
ACTTTGATCGAACAACAAAGACAGAAAAGATCTATGACGACCACCGTAAATTTCTACTGAGGATCGCC
TACGACACGTCTGGGCACCCGACTCTCTGGCTGCCAAGCAGCAAGCTGATGGCCGTCAATGTCACCTA
TTCATCCACAGGTCAAATTGCCAGCATCCAGCGAGGCACCACTAGCGAGAAAGTAGATTATGACGGAC
AGGGGAGGATCGTGTCTCGGGTCTTTGCTGATGGTAAAACATGGAGTTACACATATTTAGAAAAGTCC
ATGGTTCTTCTGCTTCATAGCCAGCGGCAGTACATCTTCGAATACGATATGTGGGACCGCCTGTCTGC
CATCACCATGCCCAGTGTGGCTCGCCACACCATGCAGACCATCCGATCCATTGGCTACTACCGCAACA
TATACAACCCCCCGGAAAGCAACGCCTCCATCATCACGGACTACAACGAGGAAGGGCTGCTTCTACAA
ACAGCTTTCTTGGGTACAAGTCGGAGGGTCTTATTCAAATACAGAAGGCAGACTAGGCTCTCAGAAAT
TTTATATGATAGCACAAGAGTCAGTTTTACCTATGATAGAACAGCAGGAGTCCTAAAGACAGTAAACC
TCCAGAGTGATGGTTTTATTTGCACCATTAGATACAGGCAAATTGGTCCCCTGATTGACAGGCAGATT
TTCCGCTTTAGTGAAGATGGGATGGTAAATGCAAGATTTGACTATAGCTATGACAACAGCTTTCGAGT
GACCAGCATGCAGGGTGTGATCAATGAAACGCCACTGCCTATTGATCTGTATCAGTTTGATGACATTT
CTGGCAAAGTTGAGCAGTTTGGAAAGTTTGGAGTTATATATTATGATATTAACCAGATCATTTCTACA
GCTGTAATGACCTATACGAAGCACTTTGATGCTCATGGCCGTATCAAGGAGATTCAATATGAGATATT
CAGGTCGCTCATGTACTGGATTACAATTCAGTATGATAACATGGGTCGGGTAACCAAGAGAGAGATTA
AAATAGGGCCCTTTGCCAACACCACCAAATATGCTTATGAATATGATGTTGATGGACAGCTCCAAACA
GTTTACCTCAATGAAAAGATAATGTGGCGGTACAACTACGATCTGAATGGAAACCTCCATTTACTGAA
CCCAAGTAACAGTGCGCGTCTGACACCCCTTCGCTATGACCTGCGAGACAGAATCACTCGACTGGGTG
ATGTTCAATATCGGTTGGATGAAGATGGTTTCCTACGTCAAAGGGGCACGGAAATCTTTGAATATAGC
TCCAAGGGGCTTCTAACTCGAGTTTACAGTAAAGGCAGTGGCTGGACAGTGATCTACCGTTATGACGG
CCTGGGAAGGCGTGTTTCTAGCAAAACCAGTCTAGGACAGCACCTGCAGTTTTTTTATGCTGACTTAA
CTTATCCCACTAGGATTACTCATGTCTACAACCATTCGAGTTCAGAAATTACCTCCCTGTATTATGAT
CTCCAAGGACATCTTTTTGCCATGGAAATCAGCAGTGGGGATGAATTCTATATTGCATCGGATAACAC
AGGGACACCACTGGCTGTGTTCAGTAGCAATGGGCTTATGCTGAAACAGATTCAGTACACTGCATATG
GGGAAATCTATTTTGACTCTAATATTGACTTTCAACTGGTAATTGGATTTCATGGTGGCCTGTATGAC
CCACTCACCAAATTAATCCACTTTGGAGAAAGAGATTATGACATTTTGGCAGGACGGTGGACAACACC
TGACATAGAAATCTGGAAAAGAATTGGGAAGGACCCAGCTCCTTTTAACTTGTACATGTTTAGGAATA
ACAACCCTGCAAGCAAAATCCATGACGTGAAAGATTACATCACAGATGTTAACAGCTGGCTGGTGACA
TTTGGTTTCCATCTGCACAATGCTATTCCTGGATTCCCTGTTCCCAAATTTGATTTAACAGAACCTTC
TTACGAACTTGTGAAGAGTCAGCAGTGGGATGATATACCGCCCATCTTCGGAGTCCAGCAGCAAGTGG
CGCGGCAGGCCAAGGCCTTCCTGTCGCTGGGGAAGATGGCCGAGGTGCAGGTGAGCCGGCGCCGGGCC
GGCGGCGCGCAGTCCTGGCTGTGGTTCGCCACGGTCAAGTCGCTGATCGGCAAGGGCGTCATGCTGGC
CGTCAGCCAGGGCCGCGTGCAGACCAACGTGCTCAACATCGCCAACGAGGACTGCATCAAGGTGGCGG
CCGTGCTCAACAACGCCTTCTACCTGGAGAACCTGCACTTCACCATCGAGGGCAAGGACACGCACTAC
TTCATCAAGACCACCACGCCCGAGAGCGACCTGGGCACGCTGCGGTTGACCAGCGGCCGCAAGGCGCT
GGAGAACGGCATCAACGTGACGGTGTCGCAGTCCACCACGGTGGTGAACGGCAGGACGCGCAGGTTCG
CGGACGTGGAGATGCAGTTCGGCGCGCTGGCGCTGCACGTGCGCTACGGCATGACCCTGGACGAGGAG
AAGGCGCGCATCCTGGAGCAGGCGCGGCAGCGCGCGCTCGCCCGGGCCTGGGCGCGCGAGCAGCAGCG
CGTGCGCGACGGCGAGGAGGGCGCGCGCCTCTGGACGGAGGGCGAGAAGCGGCAGCTGCTGAGCGCCG
GCAAGGTGCAGGGCTACGACGGGTACTACGTACTCTCGGTGGAGCAGTACCCCGAGCTGGCCGACAGC
GCCAACAACATCCAGTTCCTGCGGCAGAGCGAGATCGGCAGGAGGTAACGCCCGGGCCGCGCCCGCCG
AGCCGCTCACGCCCTGCCCACATTGTCCTGTGGCACAACCCGAGTGGGACTCTCCAACGCCCAAGAGC
CTTCCTCCCGGGGGAATGAGACTGCTGTTACGACCCACACCCACACCGCGAAAACAAGGACCGCTTTT
TTCCGAATGACCTTAAAGGTGATCGGCTTTAACGAATATGTTTACATATGCATAGCGCTGCACTCAGT
CGGACTGAACGTAGCCAGAGGAAAAAAAAATCATCAAGGACAAAGGCCTCGACCTGTTGCGCTGGGCC
GTCTGTTCCTTCTAGGCACTGTATTTAACTAACTTTAAAAAAAAAAAAAAAAAAG

| | | | |
|---|---|---|---|
| NOV40w, CG55069-08 | SEQ ID NO: 530 | 2613 aa | MW at 291899.4kD |
| Protein Sequence | | | |

MDVKERRPYCSLTKSRREKERRYTNSSADNEECRVPTQKSYSSSETLKAFDHDSSRLLYGNRVKDLVH
READEFTRQEQPASNQGQSTLQPLPPSHKQHSAQHHPSITSLNRNSLTNRRNQSPAPPAALPAELQTT
PESVQLQDSWVLGSNVPLESRHFLFKTGTGTTPLFSTATPGYTMASGSVYSPPTRPLPRNTLSRSAFK
FKKSSKYCSWKCTALCAVGVSVLLAILLSYFIAMHLFGLNWQLQQTENDTFENGKVNSDTMPTNTVSL
PSGDNGKLGGFTQENNTIDSGELDIGRRAIQEIPPGIFWRSQLFIDQPQFLKFNISLQKDALIGVYGR
KGLPPSHTQYDFVELLDGSRLIAREQRSLLETERAGRQARSVSLHEAGFIQYLDSGIWHLAFYNDGKN
AEQVSFNTIVIESVVECPRNCHGNGECVSGTCHCFPGFLGPDCSRAACPVLCSGNGQYSKGRCLCFSG
WKGTECDVPTTQCIDPQCGGRGICIMGSCACNSGYKGESCEEADCIDPGCSNHGVCIHGECHCSPGWG
GSNCEILKTMCPDQCSGHGTYLQESGSCTCDPNWTGPDCSNEICSVDCGSHGVCMGGTCRCEEGWTGP
TCNQRACHPRCAEHGTCKDGKCECSHGWNGEHCTIEGCPGLCNSNGRCTLDQNGWHCVCQPGWRGAGC
DVAMETLCTDSKDNEGDGLIDCMDPDCCLQSSCQNQPYCRGLPDPQDIISQSLQSPSQQAAKSFYDRI
SFLIGSDSTHVIPGESPFNKSLASVIRGQVLTADGTPLIGVNVSFFHYPEYGYTITRQDGMFDLVANG
GASLTLVFERSPFLTQYHTVWIPWNVFYVMDTLVMEKEENDIPSCDLSGFVRPNPIIVSSPLSTFFRS
SPEDSPIIPETQVLHEETTIPGTDLKLSYLSSRAAGYKSVLKITMTQSIIPFNLMKVHLMVAVVGRLF
QKWFPASPNLAYTFIWDKTDAYNQKVYGLSEAVVSVGYEYESCLDLTLWEKRTAILQGYELDASNMGG
WTLDKHHVLDVQNGILYKGNGENQFISQQPPVVSSIMGNGRRRSISCPSCNGQADGNKLLAPVALACG
IDGSLYVGDFNYVRRIFPSGNVTSVLELRNKDFRHSSNPAHRYYLATDPVTGDLYVSDTNTRRIYRPK
SLTGAKDLTKNAEVVAGTGEQCLPFDEARCGDGGKAVEATLMSPKGMAVDKNGLIYFVDGTMIRKVDQ
NGIISTLLGSNDLTSARPLTCDTSMHISQVRLEWPTDLAINPMDNSIYVLDNNVVLQITENRQVRIAA
GRPMHCQVPGVEYPVGKHAVQTTLESATAIAVSYSGVLYITETDEKKINRIRQVTTDGEISLVAGIPS
ECDCKNDANCDCYQSGDGYAKDAKLSAPSSLAASPDGTLYIADLGNIRIRAVSKNKPLLNSMNFYEVA
SPTDQELYIFDINGTHQYTVSLVTGDYLYNFSYSNDNDITAVTDSNGNTLRIRRDPNRMPVRVVSPDN
QVIWLTIGTNGCLKGMTAQGLELVLFTYHGNSGLLATKSDETGWTTFFDYDSEGRLTNVTFPTGVVTN
LHGDMDKAITVDIESSSREEDVSITSNLSSIDSFYTMVQDQLRNSYQIGYDGSLRIIYASGLDSHYQT
EPHVLAGTANPTVAKRNNTLPGENGQNLVEWRFRKEQAQGKVNVFGRKLRVNGRNLLSVDFDRTTKTE
KIYDDHRKFLLRIAYDTSGHPTLWLPSSKLMAVNVTYSSTGQIASIQRGTTSEKVDYDGQGRIVSRVF
ADGKTWSYTYLEKSMVLLLHSQRQYIFEYDMWDRLSAITMPSVARHTMQTIRSIGYYRNIYNPPESNA
SIITDYNEEGLLLQTAFLGTSRRVLFKYRRQTRLSEILYDSTRVSFTYDETAGVLKTVNLQSDGFICT
IRYRQIGPLIDRQIFRFSEDGMVNARFDYSYDNSFRVTSMQGVINETPLPIDLYQFDDISGKVEQFGK
FGVIYYDINQIISTAVMTYTKHFDAHGRIKEIQYEIFRSLMYWITIQYDNMGRVTKREIKIGPFANTT
KYAYEYDVDGQLQTVYLNEKINWRYNYDLNGNLHLLNPSNSARLTPLRYDLRDRITRLGDVQYRLDED
GFLRQRGTEIFEYSSKGLLTRVYSKGSGWTVIYRYDGLGRRVSSKTSLGQHLQFFYADLTYPTRITHV
YNHSSSEITSLYYDLQGHLFANEISSGDEFYIASDNTGTPLAVFSSNGLMLKQIQYTAYGEIYFDSNI
DFQLVIGFHGGLYDPLTKLIHFGERDYDILAGRWTTPDIEIWKRIGKDPAPFNLYMFRNNNPASKIHD
VKDYITDVNSWLVTFGFHLHNAIPGFPVPKFDLTEPSYELVKSQQWDDIPPIFGVQQQVARQAKAFLS
LGKMAEVQVSRRRAGGAQSWLWFATVKSLIGKGVMLAVSQGRVQTNVLMIANEDCIKVAAVLNNAFYL
ENLHFTIEGKDTHYFIKTTTPESDLGTLRLTSGRKALENGINVTVSQSTTVVNGRTRRFADVEMQFGA
LALHVRYGMTLDEEKARILEQARQRALARAWAREQQRVRDGEEGARLWTEGEKRQLLSAGKVQGYDGY
YVLSVEQYPELADSANNIQFLRQSEIGRR

| | | |
|---|---|---|
| NOV40x, CG55069-10 | SEQ ID NO: 531 | 2519 bp |
| DNA Sequence | ORF Start: at 7 | ORF Stop: at 2488 |

GGTACCAACTGGCAGCTACAGCAGACTGAAAATGACGCATTTGAGAATGGAAAAGTGAATTCTGATAC
CATGCCAACAAACACTGTGTCATTACCTTCTGGAGACAATGGAAAATTAGGTGGATTTACGCAAGAAA
ATAACACCATAGATTCCGGAGAACTTGATATTGGCCGAAGAGCAATTCAAGAGATTCCTCCCGGGATC
TTCTGGAGATCACAGCTCTTCATTGATCAGCCACAGTTTCTTAAATTCAATATCTCTCTTCAGAAGGA
TGCATTGATTGGAGTATATGGCCGGAAAGGCTTACCGCCTTCCCATACTCAGTATGACTTCGTGGAGC
TCCTGGATGGCAGCAGGCTGATTGCCAGAGAGCAGCGGAGCCTGCTTGAGACGGAGAGAGCCGGGCGG
CAGGCGAGATCCGTCAGCCTTCATGAGGCCGGCTTTATCCAGTACTTGGATTCTGGAATCTGGCATCT
GGCTTTTTATAATGATGGGAAAAATGCAGAGCAGGTGTCTTTTAATACCATTGTTATAGAGTCTGTGG
TGGAATGTCCCCGAAATTGCCATGGAAATGGAGAATGCGTTTCTGGAACTTGCCATTGTTTTCCAGGA
TTTCTGGGTCCGGATTGTTCAAGAGCCGCCTGTCCAGTGTTATGTAGTGGCAACGGGCAGTACTCCAA
GGGCCGCTGCCTGTGTTTCAGCGGCTGGAAGGGCACCGAGTGTGATGTGCCGACTACCCAGTGTATTG
ACCCACAGTGTGGGGGTCGTGGGATTTGTATCATGGGCTCCTGTGCTTGCAACTCAGGATACAAAGGA
GAAAGTTGTGAAGAAGCTGACTGTATAGACCCTGGGTGTTCTAATCATGGTGTGTGTATCCACGGGGA
ATGTCACTGCAGTCCAGGATGGGGAGGTAGCAATTGTGAAATACTGAAGACCATGTGTCCAGACCAGT
GCTCCGGCCACGGAACGTATCTTCAAGAAAGTGGCTCCTGCACGTGTGACCCTAACTGGACTGGCCCA
GACTGCTCAAACGAAATATGTTCTGTGGACTGTGGCTCACACGGCGTTTGCATGGGGGGGACGTGCCG
CTGTGAAGAAGGCTGGACGGGCCCAGCCTGTAATCAGAGAGCCTGCCACCCCCGCTGTGCCGAGCACG
GGACCTGCAAGGATGGCAAGTGTGAATGCAGCCAGGGCTGGAATGGAGAGCACTGCACTATCGCTCAC
TATTTGGATAAGATAGTTAAAGACAAGATAGGATATAAAGAGGGTTGTCCTGGTCTGTGCAACAGCAA
TGGAAGATGTACCCTGGACCAAAATGGCTGGCATTGTGCGTGCCAGCCTGGATGGAGAGGAGCAGGCT
GTGACGTAGCCATGGAGACTCTTTGCACAGATAGCAAGGACAATGAAGGAGATGGACTCATTGACTGC
ATGGATCCCGATTGCTGCCTACAGAGTTCCTGCCAGAATCAGCCCTATTGTCGGGGACTGCCGGATCC
TCAGGGCATCATTAGCCAAAGCCTTCAATCGCCTTCTCAGCAAGCTGCCAAATCCTTTTATGATCGAA
TCAGTTTCCTTATAGGATCTGATAGCACCCATGTTATACCTGGAGAAAGTCCTTTCAATAAGAGCCTT
GCATCTGTCATCAGAGGCCAAGTACTGACTGCTGATGGAACTCCACTTATTGGAGTAAATGTCTCGTT
TTTCCATTACCCAGAATATGGATATACTATTACCCGCCAGGACGGAATGTTTGACTTGGTGGCAAATG
GTGGGGCCTCTCTAACTTTGGTATTTGAACGATCCCCATTCCTCACTCAGTATCATACTGTGTGGATT
CCATGGAATGTCTTTTATGTGATGGATACCCTAGTCATGAAGAAAGAAGAGAATGACATTCCCAGCTG
TGATCTGAGTGGATTCGTGAGGCCAAATCCCATCATTGTGTCATCACCTTTATCCACCTTTTTCAGAT
CTTCTCCTGAAGACAGTCCCATCATTCCCGAAACACAGGTACTCCACGAGGAAACTACAATTCCAGGA
ACAGATTTGAAACTCTCCTACTTGAGTTCCAGAGCTGCAGGGTATAAGTCAGTTCTCAAGATCACCAT
GACCCAGTCTATTATTCCATTTAATTTAATGAAGGTTCATCTTATGGTAGCTGTAGTAGGAAGACTCT
TCCAAAAGTGGTTTCCTGCCTCACCAAACTTGGCCTATACTTTCATATGGGATAAAACAGATGCATAT
AATCAGAAAGTCTATGGTCTATCTGAAGCTGTTGTGTCAGTTGGATATGAGTATGAGTCGTGTTTGGA
CCTGACTCTGTGGGAAAAGAGGACTGCCATTCTGCAGGGCTATGAATTGGATGCGTCCAACATGGGTG
GCTGGACATTAGATAAACATCACGTGCTGGATGTACAGAACGGTATACTGTACAAGGGAAACGGGGAA
AACCAGTTCATCTCCCAGCAGCCTCCAGTCGTGAGTAGCGTCGACCCAGCAGCCTCCAGTCGTGAGTA
GCG

| | | | |
|---|---|---|---|
| NOV40x, GG55069-10 | SEQ ID NO: 532 | 827 aa | MW at 90474.8kD |
| Protein Sequence | | | |

NWQLQQTENDAFENGKVNSDTMPTNTVSLPSGDNGKLGGFTQENNTIDSGELDIGRRAIQEIPPGIFW
RSQLFIDQPQFLKFNISLQKDALIGVYGRKGLPPSHTQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACNSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGWHCACQPGWRGAGCDVAMETLCTDSKDNEGDGLIDCMD
PDCCLQSSCQNQPYCRGLPDPQGIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMKKEENDIPSCDLSGFVRPNPIIVSSPLSTFFRSSPEDSPIIPETQVLHEETTIPGTD
LKLSYLSSPAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQMGILYKGNGENQ
FISQQPPVVSS

| | | |
|---|---|---|
| NOV40y, CG55069-11 | SEQ ID NO: 533 | 2482 bp |
| DNA Sequence | ORF Start: at 11 | ORF Stop: at 2474 |

CACCTCGCGAAACTGGCAGCTACAGCAGACTGAAAATGACACATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGGCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGGCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCATTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGGGCACCGAGTGTGATGTGCCGACTACCCAGTGT
ATTGACCCACAGTGTGGGGGTCGTGGGATTTGTATCATGGGCTCTTGTGCTTGCAACTCAGGATACAA
AGGAGAAAGTTGTGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGTGCTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GTCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGAGGGTTGTCCTGGTCTGTGCAACAGCAATGGAAGATGTACCC
TGGACCAAAATGGCTGGCATTGTGTGTGCCAGCCTGGATGGAGAGGAGCAGGCTGTGACGTAGCCATG
GAGACTCTTTGCACAGATAGTAAGGACAATGAAGGAGATGGACTCATTGACTGCATGGATCCCGATTG
CTGCCTACAGAGTTCCTGCCAGAATCAGCCCTATTGTCGGGGACTGCCGGATCCTCAGGACATCATTA
GCCAAAGCCTTCAATCGCCTTCTCAGCAAGCTGCCAAATCCTTTTATGATCGAATCAGTTTCCTTATA
GGATCTGATAGCACCCATGTTATACCTGGAGAAAGTCCTTTCAATAAGAGCCTTGCATCTGTCATCAG
AGGCCAAGTACTGACTGCTGATGGAACTCCACTTATTGGAGTAAATGTCTCGTTTTTCCATTACCCAG
AATATGGATATACTATTACCCGCCAGGACGGAATGTTTGACTTGGTGGCAAATGGTGGGGCCTCTCTA
ACTTTGGTATTTGAACGATCCCCATTCCTCACTCAGTATCATACTGTGTGGATTCCATGGAATGTCTT
TTATGTGATGGATACCCTAGTCATGAAGAAAGAAGAGAATGACATTCCCAGCTGTGATCTGAGTGGAT
TCGTGAGGCCAAATCCCATCATTGTGTCATCACCTTTATCCACCTTTTTCAGATCTTCTCCTGAAGAC
AGTCCCATCATTCCCGAAACACAGGTACTCCACGAGGAAACTACAATTCCAGGAACAGATTTGAAACT
CTCCTACTTGAGTTCCAGAGCTGCAGGGTATAAGTCAGTTCTCAAGATCACCATGACCCAGTCTATTA
TTCCATTTAATTTAATGAAGGTTCATCTTATGGTAGCTGTAGTAGGAAGACTCTTCCAAAAGTGGTTT
CCTGCCTCACCAAACTTGGCCTATACTTTCATATGGGATAAAACAGATGCATATAATCAGAAAGTCTA
TGGTCTATCTGAAGCTGTTGTGTCAGTTGGATATGAGTATGAGTCGTGTTTGGACCTGACTCTGTGGG
AAAAGAGGACTGCCATTCTGCAGGGCTATGAATTGGATGCGTCCAACATGGGTGGCTGGACATTAGAT
AAACATCACGTGCTGGATGTACAGAACGGTATACTGTACAAGGGAAACGGGGAAAACCAGTTCATCTC
CCAGCAGCCTCCAGTCGTGAGTAGCCTCGAGGGC

| | | | |
|---|---|---|---|
| NOV40y, CG55069-11 | SEQ ID NO: 534 | 821 aa | MW at 89886.1kD |
| Protein Sequence | | | |

NWQLQQTENDTFENGKVNSDTMPTNTVSLPSGDNGKLGGFTQENNTIDSGELDIGRRAIQEIPPGIFW
RSQLFIDQPQFLKFNISLQKDALIGVYGRKGLPPSHTQYDFVELLDGSRLIAREQRSLLETERAGRQA
RSVSLHEAGFIQYLDSGIWHLAFYNDGKNAEQVSFNTIVIESVVECPRNCHGMGECVSGTCHCFPGFL
GPDCSRAACPVLCSGNGQYSKGRCLCFSGWKGTECDVPTTQCIDPQCGGRGICIMGSCACNSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKEGCPGLCNSNGRCTLDQNGWHCVCQPGWRGAGCDVAMETLCTDSKDNEGDGLIDCMDPDCCLQ
SSCQNQPYCRGLPDPQDIISQSLQSPSQQAAKSFYDRISFLIGSDSTHVIPGESPFNKSLASVIRGQV
LTADGTPLIGVNVSFFHYPEYGYTITRQDGMFDLVANGGASLTLVFERSPFLTQYHTVWIPWNVFYVM
DTLVMKKEENDIPSCDLSGFVRPNPIIVSSPLSTFFRSSPEDSPIIPETQVLHEETTIPGTDLKLSYL
SSRAAGYKSVLKITMTQSIIPFNLMKVHLMVAVVGRLFQKWFPASPNLAYTFIWDKTDAYNQKVYGLS
EAVVSVGYEYESCLDLTLWEKRTAILQGYELDASNMGGWTLDKHHVLDVQNGILYKGNGENQFISQQP
PVVSS

| | | |
|---|---|---|
| NOV40z, CG55069-12 | SEQ ID NO: 535 | 2500 bp |
| DNA Sequence | ORF Start: at 11 | ORF Stop: at 2492 |

CACCAAGCTTAACTGGCAGCTACAGCAGACTGAAAATGACGCATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGGCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGGCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCATTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGGGCACCGAGTGTGATGTGCCGACTACCCAGTGT
ATTGACCCACAGTGTGGGGGTCGTGGGATTTGTATCATGGGCTCCTGTGCTTGCAACTCAGGATACAA
AGGAGAAAGTTGTGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGTGCTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GCCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGACAAGATAGGATATAAAGAGGGTTGTCCTGGTCTGTGCAACA
GCAATGGAAGATGTACCCTGGACCAAAATGGCTGGCATTGTGCGTGCCAGCCTGGATGGAGAGGAGCA
GGCTGTGACGTAGCCATGGAGACTCTTTGCACAGATAGCAAGGACAATGAAGGAGATGGACTCATTGA
CTGCATGGATCCCGATTGCTGCCTACAGAGTTCCTGCCAGAATCAGCCCTATTGTCGGGGACTGCCGG
ATCCTCAGGGCATCATTAGCCAAAGCCTTCAATCGCCTTCTCAGCAAGCTGCCAAATCCTTTTATGAT
CGAATCAGTTTCCTTATAGGATCTGATAGCACCCATGTTATACCTGGAGAAAGTCCTTTCAATAAGAG
CCTTGCATCTGTCATCAGAGGCCAAGTACTGACTGCTGATGGAACTCCACTTATTGGAGTAAATGTCT
CGTTTTTCCATTACCCAGAATATGGATATACTATTACCCGCCAGGACGGAATGTTTGACTTGGTGGCA
AATGGTGGGGCCTCTCTAACTTTGGTATTTGAACGATCCCCATTCCTCACTCAGTATCATACTGTGTG
GATTCCATGGAATGTCTTTTATGTGATGGATACCCTAGTCATGAAGAAAGAAGAGAATGACATTCCCA
GCTGTGATCTGAGTGGATTCGTGAGGCCAAATCCCATCATTGTGTCATCACCTTTATCCACCTTTTTC
AGATCTTCTCCTGAAGACAGTCCCATCATTCCCGAAACACAGGTACTCCACGAGGAAACTACAATTCC
AGGAACAGATTTGAAACTCTCCTACTTGAGTTCCAGAGCTGCAGGGTATAAGTCAGTTCTCAAGATCA
CCATGACCCAGTCTATTATTCCATTTAATTTAATGAAGGTTCATCTTATGGTAGCTGTAGTAGGAAGA
CTCTTCCAAAAGTGGTTTCCTGCCTCACCAAACTTGGCCTATACTTTCATATGGGATAAAACAGATGC
ATATAATCAGAAAGTCTATGGTCTATCTGAAGCTGTTGTGTCAGTTGGATATGAGTATGAGTCGTGTT
TGGACCTGACTCTGTGGGAAAAGAGGACTGCCATTCTGCAGGGCTATGAATTGGATGCGTCCAACATG
GGTGGCTGGACATTAGATAAACATCACGTGCTGGATGTACAGAACGGTATACTGTACAAGGGAAACGG
GGAAAACCAGTTCATCTCCCAGCAGCCTCCAGTCGTGAGTAGCCTCGAGGGC

| | | | |
|---|---|---|---|
| NOV40z, CG55069-12 | SEQ ID NO: 536 | 827 aa | MW at 90474.8kD |
| Protein Sequence | | | |

NWQLQQTENDAFENGKVNSDTMPTNTVSLPSGDNGKLGGFTQENNTIDSGELDIGRRAIQEIPPGIFW
RSQLFIDQPQFLKFNISLQKDALIGVYGRKGLPPSHTQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACNSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGWHCACQPGWRGAGCDVAMETLCTDSKDNEGDGLIDCMD
PDCCLQSSCQNQPYCRGLPDPQGIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMKKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSS

| | | |
|---|---|---|
| NOV40aa, CG55069-13 | SEQ ID NO: 537 | 2541 bp |
| DNA Sequence | ORF Start: ATG at 16 | ORF Stop: TAG at 2530 |

CACCGCGGCCGCACCATGCTGCCCGGTTTGGCACTGCTCCTGCTGGCCGCCTGGACGGCTCGGGCGAA
CTGGCAGCTACAGCAGACTGAAAATGACACATTTGAGAATGGAAAAGTGAATTCTGATACCATGCCAA
CAAACACTGTGTCATTACCTTCTGGAGACAATGGAAAATTAGGTGGATTTACGCAAGAAAATAACACC
ATAGATTCCGGAGAACTTGATATTGGCCGAAGAGCAATTCAAGAGATTCCTCCCGGGATCTTCTGGAG
ATCACAGCTCTTCATTGATCAGCCACAGTTTCTTAAATTCAATATCTCTCTTCAGAAGGATGCATTGA
TTGGAGTATATGGCCQGAAAGGCTTACCGCCTTCCCATACTCAGTATGACTTCGTGGAGCTCCTGGAT
GGCAGCAGGCTGATTGCCAGAGAGCAGCGGAGCCTGCTTGAGACGGAGAGAGCCGGGCGGCAGGCGAG
ATCCGTCAGCCTTCATGAGGCCGGCTCTATCCAGTACTTGGATTCTGGAATCTGGCATCTGGCTTTTT
ATAATGATGGGAAAAATGCAGAGCAGGTGTCTTTTAATACCATTGTTATAGAGTCTGTGGTGGAATGT
CCCCGAAATTGCCATGGAAATGGAGAATGCGTTTCTGGAACTTGCCATTGTTTTCCAGGATTTCTGGG
TCCGGATTGTTCAAGAGCCGCCTGTCCAGTGTTATGTAGTGGCAACGGGCAGTACTCCAAGGGCCGCT
GCCTGTGTTTCAGCGGCTGGAAGGGCACCGAGTGTGATGTGCCGACTACCCAGTGTATTGACCCACAG
TGTGGGGGTCGTGGGATTTGTATCATGGGCTCTTGTGCTTGCAACTCAGGATACAAAGGAGAAAGTTG
TGAAGAAGCTGACTGTATAGACCCTGGGTGTTCTAATCATGGTGTGTGTATCCACGGGGAATGTCACT
GCAGTCCAGGATGGGGAGGTAGCAATTGTGAAATACTGAAGACCATGTGTCCAGACCAGTGCTCCGGC
CACGGAACGTATCTTCAAGAAAGTGGCTCCTGCACGTGTGACCCTAACTGGACTGGCCCAGACTGCTC
AAACGAAATATGTTCTGTGGACTGTGGCTCACACGGCGTTTGCATGGGGGGGACGTGTCGCTGTGAAG
AAGGCTGGACGGGCCCAGCCTGTAATCAGAGAGCCTGCCACCCCCGCTGTGCCGAGCACGGGACCTGC
AAGGATGGCAAGTGTGAATGCAGCCAGGGCTGGAATGGAGAGCACTGCACTATCGCTCACTATTTGGA
TAAGATAGTTAAAGAGGGTTGTCCTGGTCTGTGCAACAGCAATGGAAGATGTACCCTGGACCAAAATG
GCTGGCATTGTGTGTGCCAGCCTGGATGGAGAGGAGCAGGCTGTGACGTAGCCATGGAGACTCTTTGC
ACAGATAGTAAGGACAATGAAGGAGATGGACTCATTGACTGCATGGATCCCGATTGCTGCCTACAGAG
TTCCTGCCAGAATCAGCCCTATTGTCGGGGACTGCCGGATCCTCAGGACATCATTAGCCAAAGCCTTC
AATCGCCTTCTCAGCAAGCTGCCAAATCCTTTTATGATCGAATCAGTTTCCTTATAGGATCTGATAGC
ACCCATGTTATACCTGGAGAAAGTCCTTTCAATAAGAGCCTTGCATCTGTCATCAGAGGCCAAGTACT
GACTGCTGATGGAACTCCACTTATTGGAGTAAATGTCTCGTTTTTCCATTACCCAGAATATGGATATA
CTATTACCCGCCAGGACGGAATGTTTGACTTGGTGGCAAATGGTGGGGCCTCTCTAACTTTGGTATTT
GAACGATCCCCATTCCTCACTCAGTATCATACTGTGTGGATTCCATGGAATGTCTTTTATGTGATGGA
TACCCTAGTCATGAAGAAAGAAGAGAATGACATTCCCAGCTGTGATCTGAGTGGATTCGTGAGGCCAA
ATCCCATCATTGTGTCATCACCTTTATCCACCTTTTTCAGATCTTCTCCTGAAGACAGTCCCATCATT
CCCGAAACACAGGTACTCCACGAGGAAACTACAATTCCAGGAACAGATTTGAAACTCTCCTACTTGAG
TTCCAGAGCTGCAGGGTATAAGTCAGTTCTCAAGATCACCATGACCCAGTCTATTATTCCATTTAATT
TAATGAAGGTTCATCTTATGGTAGCTGTAGTAGGAAGACTCTTCCAAAAGTGGTTTCCTGCCTCACCA
AACTTGGCCTATACTTTCATATGGGATAAAACAGATGCATATAATCAGAAAGTCTATGGTCTATCTGA
AGCTGTTGTGTCAGTTGGATATGAGTATGAGTCGTGTTTGGACCTGACTCTGTGGGAAAAGAGGACTG
CCATTCTGCAGGGCTATGAATTGGATGCGTCCAACATGGGTGGCTGGACATTAGATAAACATCACGTG
CTGGATGTACAGAACGGTATACTGTACAAGGGAAACGGGGAAAACCAGTTCATCTCCCAGCAGCCTCC
AGTCGTGAGTAGCTAGGTCGACGGC

| | | | |
|---|---|---|---|
| NOV40aa, GG55069-13 | SEQ ID NO: 538 | 838 aa | MW at 91589.1kD |
| Protein Sequence | | | |

MLPGLALLLLAAWTARANWQLQQTENDTFENGKVNSDTMPTNTVSLPSGDNGKLGGFTQENNTIDSGE
LDIGRRAIQEIPPGIFWRSQLFIDQPQFLKFNISLQKDALIGVYGRKGLPPSHTQYDFVELLDGSRLI
AREQRSLLETERAGRQARSVSLHEAGSIQYLDSGIWHLAFYNDGKNAEQVSFNTIVIESVVECPRNCH
GNGECVSGTCHCFPGFLGPDCSRAACPVLCSGMGQYSKGRCLCFSGWKGTECDVPTTQCIDPQCGGRG
ICIMGSCACNSGYKGESCEEADCIDPGCSNHGVCIHGECHCSPGWGGSNCEILKTMCPDQCSGHGTYL
QESGSCTCDPNWTGPDCSNEICSVDCGSHGVCMGGTCRCEEGWTGPACNQRACHPRCAEHGTCKDGKC
ECSQGWNGEHCTIAHYLDKIVKEGCPGLCNSNGRCTLDQNGWHCVCQPGWRGAGCDVAMETLCTDSKD
NEGDGLIDCMDPDCCLQSSCQNQPYCRGLPDPQDIISQSLQSPSQQAAKSFYDRISFLIGSDSTHVIP
GESPFNKSLASVIRGQVLTADGTPLIGVNVSFFHYPEYGYTITRQDGMFDLVANGGASLTLVFERSPF
LTQYHTVWIPWNVFYVMDTLVMKKEENDIPSCDLSGFVRPNPIIVSSPLSTFFRSSPEDSPIIPETQV
LHEETTIPGTDLKLSYLSSRAAGYKSVLKITMTQSIIPFNLMKVHLMVAVVGRLFQKWFPASPNLAYT
FIWDKTDAYNQKVYGLSEAVVSVGYEYESCLDLTLWEKRTAILQGYELDASNMGGWTLDKHHVLDVQN
GILYKGNGENQFISQQPPVVSS

| | | |
|---|---|---|
| NOV40ab, CG55069-14 | SEQ ID NO: 539 | 1755 bp |
| DNA Sequence | ORF Start: ATG at 890 | ORF Stop: TAA at 1532 |

GAGAAAGGAGATTAAAAATAACCTCTGGATATTCCTCTCATGTGATCTTTATTCTGGATGAAGCATTA
GGACAGCTAATAGCCGTGTGTCACTGTGTGATTTCTTCCCTAAGACTAAGGACCCATCATTTTAGTGC
AACCTTCTTCATTTAAATGGAGAGTTGTAATTGCCAATGCTCACAGCTACTCCTGCTCCGGCAATTTG
CTGCCAGAAGTGTGTTTTCCTTTTTAAAAGGCAGTAAATTCAAGATGTTGTGGTGGATGTAGATTTTT
GCTGCAAGGAAATAACAGCTGGTGATGGAATTTCATTCTTTTGACTTCTAGATTGCCTGTGAAGAGCT
GCTTCCTCGGAAGAGCACCCTAAGGCTGGGTGGCCACTATCCTTTGCCTTGGCAGAGCCAGCCAGAAG
GCCTAGGCACAACCCGCTGTGTTTGCTGACAGCCAACCTACCCTGGAGTTCCGGAGCGGCTTCCTAGG
AAGACTGGGGAGCGGTAGAAAAATGGCTCTGCTGAGATGAGCTCTTAATTAATGCACTGAGAGCCTGC
AAGTCCCACCTCTCAACAGGAATGATTGACGTCCAAGGATACATAAATTACACTAACTGAGCTCTGCC
TCTATATAAGCTTTCCACATCCAACTCATCAGAGAAGCTAGGCTTGTACCATAACCAATACCCCTGCT
TGGCAACTCTAATGAGCAAACTGCCGCAAAATTGAGAGAGAACACACCTTTTTGATTTCCTGCTCTTC
TAAGACACAGTGATTTAGAATTTCTGTTCAAGCAAGAGAACTAAAGACTTCTTTAAAGAAGAGAAGAG
AGGCCAATGAGACTTGAACCCTGAGCCTAAGTTGTCACCAGCAGGACTGATGTGCACACAGAAGGAAT
GAAGTATGGATGTGAAAGAACGCAGGCCTTACTGCTCCCTGACCAAGAGCAGACGAGAGAAGGAACGG
CGCTACACAAATTCCTCCGCAGACAATGAGGAGTGCCGGGTACCCACACACAACTCCTACAGTTCCAG
CGAGACATTGAAAGCTTTTGATCATGATTCCTCGCGGCTGCTTTACGGCAACAGAGTGAAGGATTTGG
TTCACAGAGAAGCAGACGAGTTCACTAGACAAGGACAGAATTTTACCCTAAGGCAGTTAGGAGTTTGT
GAACCAGCAACTCGAAGAGGACTGGCATTTTGTGCGGAAATGGGGCTCCCTCACAGAGGTTACTCTAT
CAGTGCAGGGTCAGATGCTGATACTGAAAATGAAGCAGTGATGTCCCCAGAGCATGCCATGAGACTTT
GGGGCAGGGGGTTCAAATCAGGCCGCAGCTCCTGCCTGTCAAGTCGGTCCAACTCAGCCCTCACCCTG
ACAGATACGGAGCACGAAAACAAGTCCGACAGTGAGAATGGAGGGTCAAGCAGTTGGTTCGGTTTTCA
TTGGAATTTTTATGTGAGTAAAGCTTCCTGTTTGCTGCGCTTGCCTAGGATTTTCTTATCCCACAACT
ACAATGTGAACAAAGAGATGAGAGAGAAATTATGCTAATGCATTTTGGTGGATCAAATGAGTGTTTCA
TGAGACAACTCAAATTTTTGTTAGCTATATGGTGTTGGAATATAATTTCAAAGACAACTAAGCCCTAA
AATAGGAGATTTATTTAAAACATAACTTTTCCTTGAATGAAAGGATGTTTTTGTTCTTTCTCTGACAA
ATATGATTTGAGAATAAAAGACCTGCCCGGGCAGCCGCTCGAGCCCTATAGTGAG

| | | | |
|---|---|---|---|
| NOV40ab, CG55069-14 | SEQ ID NO: 540 | 214 aa | MW at 24449.8kD |
| Protein Sequence | | | |

MDVKERRPYCSLTKSRREEKRRYTNSSADNEECRVPTHNSYSSSETLKAFDHDSSRLLYGNRVKDLVH
READEFTRQGQNFTLRQLGVCEPATRRGLAFCAEMGLPHRGYSISAGSDADTENEAVMSPERAMRLWG
RGFKSGRSSCLSSRSNSALTLTDTEHENKSDSENGGSSSWFGFHWNFYVSKASCLLRLPRIFLSHNYN
VNKEMREKLC

| | | |
|---|---|---|
| NOV40ac, CG55069-15 | SEQ ID NO: 541 | 768 bp |
| DNA Sequence | ORF Start: ATG at 65 | ORF Stop: TAA at 707 |

AGACTTGAACCCTGAGCCTAAGTTGTCACCAGCAGGACTGATGTGCACACAGAAGGAATGAAGTATGG
ATGTGAAAGAACGCAGGCCTTACTGCTCCCTGACCAAGAGCAGACGAGAGAAGGAACGGCGCTACACA
AATTCCTCCGCAGACAATGAGGAGTGCCGGGTACCCACACAGAAGTCCTACAGTTCCAGCGAGACATT
GAAAGCTTTTGATCATGATTCCTCGCGGCTGCTTTACGGCAACAGAGTGAAGGATTTGGTTCACAGAG
AAGCAGACGAGTTCACTAGACAAGGACAGAATTTTACCCTAAGGCAGTTAGGAGTTTGTGAACCAGCA
ACTCGAAGAGGACTGGCATTTTGTGCGGAAATGGGGCTCCCTCACAGAGGTTACTCTATCAGTGCAGG
GTCAGATGCTGATACTGAAAATGAAGCAGTGATGTCCCCAGAGCATGCCATGAGACTTTGGGGCAGGG
GGGTCAAATCAGGCCGCAGCTCCTGCCTGTCAAGTCGGTCCAACTCAGCCCTCACCCTGACAGATACG
GAGCACGAAAACAAGTCCGACAGTGAGAATGGAGGGTCAAGCAGTTGGTTCGGTTTTCATTGGAATTT
TTATGTGGGTAAAGCTTCCTGTTTGCTGCGCTTGCCTAGGATTTTCTTATCCCACAACTACAATGTGA
ACAAAGAGATGAGAGAGAAATTATGCTAATGCATTTTGGTGGATCAATGCTAATGCATTTTGGTGGAT
CAATGCTAATGCATTTTGGT

| | | | |
|---|---|---|---|
| NOV40ac, CG55069-15 | SEQ ID NO: 542 | 214 aa | MW at 24376.8kD |
| Protein Sequence | | | |

MDVKERRPYCSLTKSRREEKRRYTNSSADNEECRVPTHNSYSSSETLKAFDHDSSRLLYGNRVKDLVH
READEFTRQGQNFTLRQLGVCEPATRRGLAFCAEMGLPHRGYSISAGSDADTENEAVMSPERAMRLWG
RGFKSGRSSCLSSRSNSALTLTDTEHENKSDSENGGSSSWFGFHWNFYVSKASCLLRLPRIFLSHNYN
VNKEMREKLC

| | | |
|---|---|---|
| NOV40ad, SNP13374479 of | SEQ ID NO: 543 | 8657 bp |
| CG55069-01, DNA Sequence | ORF Start: ATG at 151 | ORF Stop: TAA at 8326 |
| | SNP Pos: 465 | SNP Change: C to T |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTTCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGTATCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCAGGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40ad, SNP13374479 of | SEQ ID NO: 544 | 2725 aa | MW at 303959.6kD |
| CG55069-01, Protein | SNP Pos: 105 | | SNP Change: Leu to Leu |
| Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLMWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNOPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGAIALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40ae, SNP13382453 of | SEQ ID NO: 545 | 8657 bp |
| CG55069-01, DNA Sequence | ORF Start: ATG at 151 | ORF Stop: TAA at 8326 |
| | SNP Pos: 973 | SNP Change: T to C |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCACCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGThTCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40ae, SNP13382453 of | SEQ ID NO: 546 | 2725 aa | MW at 303969.7kD |
| CG55069-01, Protein | SNP Pos: 275 | | SNP Change: Ser to Pro |
| Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MAPGSVYSPPTRPLPRNTLSRSAFKFKKSSKYCSWKCTALCAVGVSVLLAILLSYFIAMHLFGLMWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNOPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGAIALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40af, SNP13382454 of | SEQ ID NO: 547 | 8657 bp |
| CG55069-01, DNA Sequence | ORF Start: ATG at 151 | ORF Stop: TAA at 8326 |
| | SNP Pos: 1953 | SNP Change: C to T |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCTTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGThTCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40af, SNP13382454 of | SEQ ID NO: 548 | 2725 aa | MW at 303959.6kD |
| CG55069-01, Protein | SNP Pos: 601 | | SNP Change: Ser to Ser |
| Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLMWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNOPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGAIALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40ag, SNP13382455 of | SEQ ID NO: 549 | 8657 bp |
| CG55069-01, DNA Sequence | ORF Start: ATG at 151 | ORF Stop: TAA at 8326 |
| | SNP Pos: 2559 | SNP Change: C to T |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGThTCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGTAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40ag, SNP13382455 of | SEQ ID NO: 550 | 2725 aa | MW at 303959.6kD |
| CG55069-01, Protein | SNP Pos: 803 | | SNP Change: Ser to Ser |
| Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLMWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNOPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGAIALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| NOV40ah, SNP13378354 of | SEQ ID NO: 551 | 8657 bp |
| CG55069-01, DNA Sequence | ORF Start: ATG at 151 | ORF Stop: TAA at 8326 |
| | SNP Pos: 7770 | SNP Change: C to T |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGATTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGThTCTTCAAGAAAGTGGCTCCTGCACGTGTGACCCTAACTGGA
CTGGCCCAGACTGCTCAAACGAAATATGTTCTGTGGACTGTGGCTCACACGGCGTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTTATACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTAGCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTCTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTCTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTTAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCCTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| NOV40ah, SNP13378354 of | SEQ ID NO: 552 | 2725 aa | MW at 303959.6kD |
| CG55069-01, Protein | SNP Pos: 2540 | | SNP Change: Leu to Leu |
| Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLMWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVIESVVECPRNCHGNGECVSGTCHCFPGFL
GPDCSRAACPVLCSGNGQYSKGRCLCFSGWKGTECDVPTTQCIDPQCGGRGICIMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNOPYCRGLPDPQDIISQSLQSPSQQAAKSFYDRISFLIGSDSTHVIPGESPFNKSLAS
VIRGQVLTADGTPLIGVNVSFFHYPEYGYTITRQDGMFDLVANGGASLTLVFERSPFLTQYHTVWIPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSIIPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYITETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWTTFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTTSEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAIP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGAIALHVRYGMTLDEEKARILEQARQ
RALARAWAREQQRVRDGEEGARLWTEGEKRQLLSAGKVQGYDGYYVLSVEQYPELADSANNIQFLRQS
EIGRR

A ClustalW comparison of the above protein sequences yields the following sequence alignment shown in Table 40B.

TABLE 40B
Comparison of the NOV40 protein sequences.

| | |
|---|---|
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | MDVKERRPYCSLTKSRREKERRYTNSSADNEECRVPTQKSYSSSETLKAFDHDSSRLLYG |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | NRVKDLVHREADEFTRQEQPASNQGQSTLQPLPPSHKQHSAQHHPSITSLNRNSLTNRRN |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | QSPAPPAALPAELQTTPESVQLQDSNVLGSNVPLESRHFLFKTGTGTTPLFSTATPGYTN |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | ASGSVYSPPTRPLPRNTLSRSAFKFKKSSKYCSWKCTALCAVGVSVLLAILLSYFIAMHL |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | FGLNWQLQQTENDTFENGKVNSDTMPTNTVSLPSGDNGKLGGFTQENNTIDSGELDIGRR |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | AIQEIPPGIFWRSQLFIDQPQFLKFNISLQKDALIGVYGRKGLPPSHTQYDFVELLDGSR |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40g | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | LIAREQRSLLETERAGRQARSVSLHEAGFIQYLDSGIWHLAFYNDGKNAEQVSFNTIVIE |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | SVVECPRNCHGNGECVSGTCHCFPGFLGPDCSRAACPVLCSGNGQYSKGRCLCFSGWKGT |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | ECDVPTTQCIDPQCGGRGICIMGSCACNSGYKGESCEEADCIDPGCSNHGVCIHGECHCS |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | PGWGGSNCEILKTMCPDQCSGHGTYLQESGSCTCDPNWTGPDCSNEICSVDCGSHGVCMG |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | GTCRCEEGWTGPTCNQRACHPRCAEHGTCKDGKCECSHGWNGEHCTIEGCPGLCNSNGRC |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | TLDQNGWHCVCQPGWRGAGCDVANETLCTDSKDNEGDGLIDCMDPDCCLQSSCQNQPYCR |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | GLPDPQDIISQSLQSPSQQAAKSFYDRISFLIGSDSTHVIPGESPFNKSLASVIRGQVLT |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | ADGTPLIGVNVSFFNYPEYGYTITRQDGMFDLVANGGASLTLVFERSPFLTQYHTVWIPW |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | NVFYVMDTLVMEKEENDIPSCDLSGFVRPNPIIVSSPLSTFFRSSPEDSPIIPETQVLHE |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | ETTIPGTDLKLSYLSSRAAGYKSVLKITMTQSIIPFNLMKVHLMVAVVGRLFQKWFPASP |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | NLAYTFIWDKTDAYNQKVYGLSEAVVSVGYEYESCLDLTLWEKRTAILQGYELDASNMGG |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | WTLDKHHVLDVQNGILYKGNGEMQFISQQPPVVSSIMGNGRRRSISCPSCNGQADGNKLL |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | APVALACGIDGSLYVGDFNYVRRIFPSGNVTSVLELRNKDFRHSSNPAHRYYLATDPVTG |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | DLYVSDTNTRRIYRPKSLTGAKDLTKNAEVVAGTGEQCLPFDEARCGDGGKAVEATLMSP |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | KGMAVDKNGLIYFVDGTMIRKVDQNGIISTLLGSNDLTSARPLTCDTSMHISQVRLEWPT |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | DLAINPMDNSIYVLDNNVVLQITENRQVRIAAGRPMHCQVPGVEYPVGKHAVQTTLESAT |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | AIAVSYSGVLYITETDEKKINRIRQVTTDGEISLVAGIPSECDCKHDANCDCYQSGDGYA |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | KDAKLSAPSSLAASPDGTLYIADLGNIRIRAVSKNKPLLNSMNFYEVASPTDQELYIFDI |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | NGTHQYTVSLVTGDYLYNFSYSNDNDITAVTDSNGNTLRIRRDPNRMPVRVVSPDNQVIW |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ------------------------------------------------------------ |
| NOV40v | ------------------------------------------------------------ |
| NOV40w | LTIGTNGCLKGMTAQGLELVLFTYHGNSGLLATKSDETGWTTFFDYDSEGRLTNVTFPTG |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ---------MDVKERRPYCSLTKSRREKBRRYTNSSADNEECRVPTQKSYSSSETLKAFD |
| NOV40v | ------------------------------------------------------------ |
| NOV40w | VVTNLHGDMDKAITVDIESSSREEDVSITSNLSSIDSFYTMVQDQLRNSYQIGYDGSLRI |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | HDSSRLLYGNRVKDLVHREADEFTRQGQNFTLRQLGVCEPATRRGLAFCAENGLPHRGYS |
| NOV40v | ------------------------------------------------------------ |
| NOV40w | IYASGLDSHYQTEPHVLAGTANPTVAKRNMTLPGENGQNLVEWRFRKEQAQGKVNVFGRK |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40 k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | ISAGSDADTENEAVMSPEHAMRLWGRGVKSGRSSCLSSRSNSALTLTDTEHENKSDSENE |
| NOV40v | ------------------------------------------------------------ |
| NOV40w | LRVNGRNLLSVDFDRTTKTEKIYDDHRKFLLRIAYDTSGHPTLWLPSSKLMAVNVTYSST |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | QPASNQGQSTLQPLPPSHKQHSAQHHPSITSLNRNSLTNRRNQSPAPPAALPAELQTTPE |
| NOV40v | ------------------------------------------------------------ |
| NOV40w | GQIASIQRGTTSEKVDYDGQGRIVSRVFADGKTWSYTYLEKSMVLLLHSQRQYIFEYDMW |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | --------------------MDVKERRPYCSLTKSRREKERRYTNSSADNEECRVPTQK |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | SVQLQDSWVLGSNVPLESRHFLFKTGTGTTPLFSTATPGYTMASGSVYSPPTRPLPRNTL |
| NOV40v | ------------------------------------------------------------ |
| NOV40w | DRLSAITNPSVARHTNQTIRSIGYYRNIYNPPESNASIITDYNEEGLLLQTAFLGTSRRV |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | SYSSSETLKAFDHDSSRLLYGNRVKDLVHREADEFTRQGQNFTLRQLGVCEPATRRGLAF |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------MDVKER |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | SRSAFKFKKSSKYCSWKCTALCAVGVSVLLAILLSYFIAMHLFGLNWQLQQTENDTFENG |
| NOV40v | ------------------------------------------------------------ |
| NOV40w | LFKYRRQTRLSEILYDSTRVSFTYDETAGVLKTVNLQSDGFICTIRYRQIGPLIDRQIFR |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | CAEMGLPHRGYSISAGSDADTENEAVMSPEHANRLWGRGVKSGRSSCLSSRSNSALTLTD |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | RPYCSLTKSRREKERRYTNSSADNEECRVPTQKSYSSSETLKAFDHDSSRLLYGNRVKDL |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | KVNSDTMPTNTVSLPSGDNGKLGGFTQENNTIDSGELDIGRRAIQEIPPGIFWRSQLFID |
| NOV40v | ------------------------------------------------------------ |
| NOV40w | FSEDGMVNARFDYSYDNSFRVTSMQGVINETPLPIDLYQFDDISGKVEQFGKFGVIYYDI |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | TEHENKSDSENEQPASNQGQSTLQPLPPSHKQHSAQHHPSITSLNRNSLTNRRNQSPAPP |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | VHREADEFTRQEQPASNQGQSTLQPLPPSHKQHSAQHHPSITSLNRNSLTNRRNQSPAPP |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | QPQFLKFNISLQKDALIGVYGRKKLPPSHTQSSPQYDFVELLDGSRLIAREQRSLLETER |
| NOV40v | ------------------------------------------------------------ |
| NOV40w | NQIISTAVMTYTKHFDAHGRIKEIQYEIFRSLMYWITIQYDNMGRVTKREIKIGPFANTT |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | AALPAELQTTPESVQLQDSWVLGSNVPLESRHFLFKTGTGTTPLFSTATPGYTMASGSVY |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | AALPAELQTTPESVQLQDSWVLGSNVPLESRHFLFKTGTGTTPLFSTATPGYTMASGSVY |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ------------------------------------------------------------ |
| NOV40t | ------------------------------------------------------------ |
| NOV40u | AGRQARSVSLHEAGFIQYLDSGIWHLAFYNDGKNAEQVSFNTIVIESVVECPRNCHGNGE |
| NOV40v | ------------------------------------------------------------ |
| NOV40w | KYAYEYDVDGQLQTVYLNEKIMWRYNYDLNGNLHLLNPSNSARLTPLRYDLRDRITRLGD |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | SPPTRPLPRNTLSRSAFKFKKSSKYCSWKCTALCAVGVSVLLAILLSYFIAMHLFGLNWQ |
| NOV40b | ------------------------------------------------------------ |
| NOV40c | -------------------------------------------------------GTNWQ |
| NOV40d | -------------------------------------------------------GTNWQ |
| NOV40e | -------------------------------------------------------GTNWQ |
| NOV40f | ------------------------------------------------------TKLNWQ |
| NOV40g | ------------------------------------------------------------ |
| NOV40h | ------------------------------------------------------------ |
| NOV40i | SPPTRPLPRNTLSRSAFKFKKSSKYCSWKCTALCAVGVSVLLAILLSYFIAMHLFGLNWQ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ---------------------------------------------------------NWQ |
| NOV40r | ------------------------------------------------------TSRNWQ |
| NOV40s | ------------------------------------------------------------ |
| NOV40t | ------------------------------------------------------------ |
| NOV40u | CVSGTCHCFPGFLGPDCSRAACPVLCSGNGQYSKGRCLCFSGWKGTECDVPTTQCIDPQC |
| NOV40v | ------------------------------------------------------------ |
| NOV40w | VQYRLDEDGFLRQRGTEIFEYSSKGLLTRVYSKGSGWTVIYRYDGLGRRVSSKTSLGQHL |
| NOV40x | ---------------------------------------------------------NWQ |
| NOV40y | ---------------------------------------------------------NWQ |
| NOV40z | ---------------------------------------------------------NWQ |
| NOV40aa | ----------------------------------------MLPGLALLLLAAWTARANWQ |
| NOV40ab | ------------------------------------------------------------ |
| NOV40ac | ------------------------------------------------------------ |
| NOV40a | LQQTENDTFENGKVNSDTMPTNTVSLPSGDNGKLGGFTQENNTIDSGELDIGRRAIQEIP |
| NOV40b | ------------------------------------------------------------ |
| NOV40c | LQQTENDTFENGKVNSDTMPTNTVSLPSGDNGKLGGFTQENNTIDSGELDIGRRAIQEIP |
| NOV40d | LQQTENDAFENGKVNSDTMPTNTVSLPSGDNGKLGGFTQENNTIDSGELDIGRRAIQEIP |
| NOV40e | LQQTENDTFENGKVNSDTMPTNTVSLPSGDNGKLGGFTQENNTIDSGELDIGRRAIQEIP |
| NOV40f | LQQTENDAFENGKVNSDTMPTNTVSLPSGDNGKLGGFTQENNTIDSGELDIGRRAIQEIP |
| NOV40g | ------------------------------------------------------------ |
| NOV40h | ------------------------------------------------------------ |
| NOV40i | LQQTENDTFENGKVNSDTMPTNTVSLPSGDNGKLGGFTQENNTIDSGELDIGRRAIQEIP |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | LQQTENDTFENGKVNSDTMPTNTVSLPSGDNGKLGGFTQENNTIDSGELDIGRRAIQEIP |
| NOV40r | LQQTENDTFENGKVNSDTMPTNTVSLPSGDNGKLGGFTQENNTIDSGELDIGRRAIQEIP |
| NOV40s | ------------------------------------------------------------ |
| NOV40t | ------------------------------------------------------------ |
| NOV40u | GGRGICTMGSCACSSGYKGESCEEADCIDPGCSNHGVCIHGECHCSPGWGGSNCEILKTM |
| NOV40v | ------------------------------------------------------------ |
| NOV40w | QFFYADLTYPTRITHVYNHSSSEITSLYYDLQGHLFANEISSGDEFYIASDNTGTPLAVF |
| NOV40x | LQQTENDAFENGKVNSDTMPTNTVSLPSGDNGKLGGFTQENNTIDSGELDIGRRAIQEIP |
| NOV40y | LQQTENDTFENGKVNSDTMPTNTVSLPSGDNGKLGGFTQENNTIDSGELDIGRRAIQEIP |
| NOV40z | LQQTENDAFENGKVNSDTMPTNTVSLPSGDNGKLGGFTQENNTIDSGELDIGRRAIQEIP |
| NOV40aa | LQQTENDTFENGKVNSDTMPTNTVSLPSGDNGKLGGFTQENNTIDSGELDIGRRAIQEIP |
| NOV40ab | ------------------------------------------------------------ |
| NOV40ac | ------------------------------------------------------------ |
| NOV40a | PGIFWRSQLFIDQPQFLKFNISLQKDALIGVYGRKKLPPSHTQSSPQYDFVELLDGSRLI |
| NOV40b | ------------------------------------------------------------ |
| NOV40c | PGIFWRSQLFIDQPQFLKFNISLQKDALIGVYGRKGLPPSHT----QYDFVELLDGSRLI |
| NOV40d | PGIFWRSQLFIDQPQFLKFNISLQKDALIGVYGRKGLPPSHT----QYDFVELLDGSRLI |
| NOV40e | PGIFWRSQLFIDQPQFLKFNISLQKDALIGVYGRKGLPPSHT----QYDFVELLDGSRLI |
| NOV40f | PGIFWRSQLFIDQPQFLKFNISLQKDALIGVYGRKGLPPSHT----QYDFVELLDGSRLI |
| NOV40g | ------------------------------------------------------------ |
| NOV40h | ------------------------------------------------------------ |
| NOV40i | PGIFWRSQLFIDQPQFLKFNISLQKDALIGVYGRKGLPPSNT----QYDFVELLDGSRLI |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | PGIFWRSQLFIDQPQFLKFNISLQKDALIGVYGRKGLPPSHT----QYDFVELLDGSRLI |
| NOV40r | PGIFWRSQLFIDQPQFLKFNISLQKDALIGVYGRKGLPPSHT----QYDFVELLDGSRLI |
| NOV40s | ------------------------------------------------------------ |
| NOV40t | ------------------------------------------------------------ |
| NOV40u | CPDQCSGHGTYLQESGSCTCDPNWTGPDCSNEICSVDCGSHG----VCMGGTCRCEEGWT |
| NOV40v | ------------------------------------------------------------ |
| NOV40w | SSNGLMLKQIQYTAYGEIYFDSNIDFQLVIGFHGGLYDPLTKLIHFGERDYDILAGRWTT |
| NOV40x | PGIFWRSQLFIDQPQFLKFNISLQKDALIGVYGRKGLPPSHT----QYDFVELLDGSRLI |
| NOV40y | PGIFWRSQLFIDQPQFLKFNISLQKDALIGVYGRKGLPPSHT----QYDFVELLDGSRLI |
| NOV40z | PGIFWRSQLFIDQPQFLKFNISLQKDALIGVYGRKGLPPSHT----QYDFVELLDGSRLI |
| NOV40aa | PGIFWRSQLFIDQPQFLKFNISLQKDALIGVYGRKGLPPSHT----QYDFVELLDGSRLI |
| NOV40ab | ------------------------------------------------------------ |
| NOV40ac | ------------------------------------------------------------ |
| NOV40a | AREQRSLLETERAGRQARSVSLHEAGFIQYLDSGIWHLAFYNDGKNAEQVSFNTIVIESV |
| NOV40b | ------------------------------------------------------------ |
| NOV40c | AREQRSLLETERAGRQARSVSLHEAGFIQYLDSGIWHLAFYNDGKNAEQVSFNTIVIESV |
| NOV40d | AREQRSLLETERAGRQARSVSLHEAGFIQYLDSGIWHLAFYNDGKNAEQVSFNTIVIESV |
| NOV40e | AREQRSLLETERAGRQARSVSLHEAGFIQYLDSGIWHLAFYNDGKNAEQVSFNTIVIESV |
| NOV40f | AREQRSLLETERAGRQARSVSLHEAGFIQYLDSGIWHLAFYNDGKNAEQVSFNTIVIESV |
| NOV40g | ------------------------------------------------------------ |
| NOV40h | ------------------------------------------------------------ |
| NOV40i | AREQRSLLETERAGRQARSVSLHEAGFIQYLDSGIWHLAFYNDGKNAEQVSFNTIVIESV |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | AREQRSLLETERAGRQARSVSLHEAGFIQYLDSGIWHLAFYNDGKNAEQVSFNTIVIESV |
| NOV40r | AREQRSLLETERAGRQARSVSLHEAGFIQYLDSGIWHLAFYNDGKNAEQVSFNTIVIESV |
| NOV40s | ------------------------------------------------------------ |
| NOV40t | ------------------------------------------------------------ |
| NOV40u | GPACNQRACHPRCAEHGTCKDGKCECSQGWNGEHCTIAHYLDKIVKDKIGYKEGCPGLCN |
| NOV40v | ------------------------------------------------------------ |
| NOV40w | PDIEIWKRIGKDPAPFNLYMFRNNNPASKIHDVKDYITDVNSWLVTFGFHLHNAIPGFPV |
| NOV40x | AREQRSLLETERAGRQARSVSLHEAGFIQYLDSGIWHLAFYNDGKNAEQVSFNTIVIESV |
| NOV40y | AREQRSLLETERAGRQARSVSLHEAGFIQYLDSGIWHLAFYNDGKNAEQVSFNTIVIESV |
| NOV40z | AREQRSLLETERAGRQARSVSLHEAGFIQYLDSGIWHLAFYNDGKNAEQVSFNTIVIESV |
| NOV40aa | AREQRSLLETERAGRQARSVSLHEAGFIQYLDSGIWHLAFYNDGKNAEQVSFNTIVIESV |
| NOV40ab | ------------------------------------------------------------ |
| NOV40ac | ------------------------------------------------------------ |
| NOV40a | VECPRNCHGNGECVSGTCHCFPGFLGPDCSRAACPVLCSGNGQYSKGRCLCFSGWKGTEC |
| NOV40b | --CPRNCHGNGECVSGTCHCFPGFLGPDCSRAACPVLCSGNGQYSKGRCLCFSGWKGTEC |
| NOV40c | VECPRNCHGNGECVSGTCHCFPGFLGPDCSRAACPVLCSGNGQYSKGRCLCFSGWKGTEC |
| NOV40d | VECPRNCHGNGECVSGTCHCFPGFLGPDCSRAACPVLCSGNGQYSKGRCLCFSGWKGTEC |
| NOV40e | VECPRNCHGNGECVSGTCHCFPGFLGPDCSRAACPVLCSGNGQYSKGRCLCFSGWKGTEC |
| NOV40f | VECPRNCHGNGECVSGTCHCFPGFLGPDCSRAACPVLCSGNGQYSKGRCLCFSGWKGTEC |
| NOV40g | --TSRTEPSYELVKSQQWDDIPPIFGVQQQVARQAKAFLSLG---KMAEVQVSRRRAGGA |
| NOV40h | --------------------------SMDVKERRPYCSLTKSRREKERRYTNSSADNEEC |
| NOV40i | VECPRNCHGNGECVSGTCHCFPGFLGPDCSRAACPVLCSGNGQYSKGRCLCFSGWKGTEC |
| NOV40j | -----KLDQNG-----------------------------------GHCVCQPGWRGAGC |
| NOV40k | -----KLDQNG-----------------------------------GHCVCQPGWRGAGC |
| NOV40l | -----KLDQNG-----------------------------------GHCVCQPGWRGAGC |
| NOV40m | -----KLDQNG-----------------------------------GHCVCQPGWRGAGC |
| NOV40n | -----KLDQNG-----------------------------------GHCVCQPGWRGAGC |
| NOV40o | -----KLDQNG-----------------------------------GHCVCQPGWRGAGC |
| NOV40p | -----KLDQNG-----------------------------------GHCVCQPGWRGAGC |
| NOV40q | VECPRNCHGNGECVSGTCHCFPGFLGPDCSRAACPVLCSGNGQYSKGRCLCFSGWKGTEC |
| NOV40r | VECPRNCHGNGECVSGTCHCFPGFLGPDCSRAACPVLCSGNGQYSKGRCLCFSGWKGTEC |
| NOV40s | ---------------------------MDVKERRPYCSLTKSRREKERRYTNSSADNEEC |
| NOV40t | --CPRNCHGNGECVSGTCHCFPGFLGPDCSRAACPVLCSGNGQYSKGRCLCFSGWKGTEC |
| NOV40u | SNGRCTLDQNG-----------------------------------GHCVCQPGWRGAGC |
| NOV40v | -------DQNG-----------------------------------GHCVCQPGWRGAGC |
| NOV40w | PKFDLTEPSYELVKSQQWDDIPPIFGVQQQVARQAKAFLSLG---KMAEVQVSRRRAGGA |
| NOV40x | VECPRNCHGNGECVSGTCHCFPGFLGPDCSRAACPVLCSGNGQYSKGRCLCFSGWKGTEC |
| NOV40y | VECPRNCHGNGECVSGTCHCFPGFLGPDCSRAACPVLCSGNGQYSKGRCLCFSGWKGTEC |
| NOV40z | VECPRNCHGNGECVSGTCHCFPGFLGPDCSRAACPVLCSGNGQYSKGRCLCFSGWKGTEC |
| NOV40aa | VECPRNCHGNGECVSGTCHCFPGFLGPDCSRAACPVLCSGNGQYSKGRCLCFSGWKGTEC |
| NOV40ab | ---------------------------MDVKERRPYCSLTKSRREKERRYTNSSADNEEC |
| NOV40ac | ---------------------------MDVKERRPYCSLTKSRREKERRYTNSSADNEEC |
| NOV40a | DVPTTQCIDPQCGGRGICINGSCACSSGYKGESCEEADCIDPGCSNHGVCIHGECHCSPG |
| NOV40b | DVPTTQCIDPQCGGRGICIMGSCACNSGYKGESCEEADCIDPGCSNHGVCIHGECHCSPG |
| NOV40c | DVPTTQCIDPQCGGRGICIMGSCACNSGYKGESCEEADCIDPGCSNHGVCIHGECHCSPG |
| NOV40d | DVPTTQCIDPQCGGRGICIMGSCACNSGYKGESCEEADCIDPGCSNHGVCIHGECHCSPG |
| NOV40e | DVPTTQCIDPQCGGRGICIMGSCACNSGYKGESCEEADCIDPGCSNHGVCIHGECHCSPG |
| NOV40f | DVPTTQCIDPQCGGRGICIMGSCACNSGYKGESCEEADCIDPGCSNHGVCIHGECHCSPG |
| NOV40g | QSWLWFATVKSLIGKGVMLAVSQG-RVQTNVLNIANEDCIKVAAVLNNAFYLENLHFTIE |
| NOV40h | RVPTQKSYSSSE----------------TLKAFDHDSSRLLYGNRVKDLVHREADEFTRQ |
| NOV40i | DVPTTQCIDPQCGGRGICIMGSCACNSGYKGKSCEEADCIDPGCSNHGVCIHGECHCSPG |
| NOV40j | DVAMETLCTDSK-----------------DNEGDGLIDCMDPDCCLQSSCQN-QPYCRGL |
| NOV40k | DVAMETLCTDSK-----------------DNEGDGLIDCMDPDCCLQSSCQN-QPYCRGL |
| NOV40l | DVAMETLCTDSK-----------------DNEGDGLIDCMDPDCCLQSSCQN-QPYCRGL |
| NOV40m | DVANETLCTDSK-----------------DNEGDGLIDCMDPDCCLQSSCQN-QPYCRGL |
| NOV40n | DVAMETLCTDSK-----------------DNEGDGLIDCMDPDCCLQSSCQN-QPYCRGL |
| NOV40o | DVAMETLCTDSK-----------------DNEGDGLIDCMDPDCCLQSSCQN-QPYCRGL |
| NOV40p | DVAMETLCTDSK-----------------DNEGDGLIDCMDPDCCLQSSCQN-QSYCRGL |
| NOV40q | DVPTTQCIDPQCGGRGICIMGSCACNSGYKGESCEEADCIDPGCSNHGVCIHGECHCSPG |
| NOV40r | DVPTTQCIDPQCGGRGICIMGSCACNSGYKGESCEEADCIDPGCSNHGVCIHGECHCSPG |
| NOV40s | RVPTQKSYSSSE----------------TLKAFDHDSSRLLYGNRVKDLVHREADEFTRQ |
| NOV40t | DVPTTQCIDPQCGGRGICIMGSCACNSGYKGESCEEADCIDPGCSNHGVCIHGECHCSPG |
| NOV40u | DVAMETLCTDSK-----------------DNEGDGLIDCMDPDCCLQSSCQN-QPYCRGL |
| NOV40v | DVAMETLCTDSK-----------------DNEGDGLIDCMDPDCCLQSSCQN-QPYCRGL |
| NOV40w | QSWLWFATVKSLIGKGVMLAVSQG-RVQTNVLNIANEDCIKVAAVLNNAFYLENLHFTIE |
| NOV40x | DVPTTQCIDPQCGGRGICIMGSCACNSGYKGESCEEADCIDPGCSNHGVCIHGECHCSPG |
| NOV40y | DVPTTQCIDPQCGGRGICIMGSCACNSGYKGESCEEADCIDPGCSNHGVCIHGECHCSPG |
| NOV40z | DVPTTQCIDPQCGGRGICIMGSCACNSGYKGESCEEADCIDPGCSNHGVCIHGECHCSPG |
| NOV40aa | DVPTTQCIDPQCGGRGICIMGSCACNSGYKGESCEEADCIDPGCSNHGVCIHGECHCSPG |
| NOV40ab | RVPTHNSYSSSE----------------TLKAFDHDSSRLLYGNRVKDLVHREADEFTRQ |
| NOV40ac | RVPTQKSYSSSE----------------TLKAFDHDSSRLLYGNRVKDLVHREADEFTRQ |
| NOV40a | WGGSNCEILKTMCPDQCSGHGTYLQESGSCTCDPNWTGPDCSNEICSVDCGSHGVCMGGT |
| NOV40b | WGGSNCEILKTMCPDQCSGHGTYLQESGSCTCDPNWTGPDCSNEICSVDCGSHGVCMGGT |
| NOV40c | WGGSNCEILKTMCPDQCSGHGTYLQESGSCTCDPNWTGPDCSNEICSVDCGSHGVCMGGT |
| NOV40d | WGGSNCEILKTMCPDQCSGHGTYLQESGSCTCDPNWTGPDCSNEICSVDCGSHGVCMGGT |
| NOV40e | WGGSNCEILKTMCPDQCSGHGTYLQESGSCTCDPNWTGPDCSNEICSVDCGSHGVCMGGT |
| NOV40f | WGGSNCEILKTMCPDQCSGHGTYLQESGSCTCDPNWTGPDCSNEICSVDCGSHGVCMGGT |
| NOV40g | -GKDTHYFIKTTTPESDLGTLRLTSGRKALENGINVTVSQSTTVVNGRTRRFADVEMQFG |
| NOV40h | EQPASNQGQSTLQPLPPSHKQHSAQHHPSITSLNRNSLTNRRNQSPAPPAALPAELQTTP |
| NOV40i | WGGSNCEILKTMCPDQCSGHGTYLQESGSCTCDPNWTGPDCSNEICSVDCGSHGVCMGGT |
| NOV40j | PDPQDIISQSLQSPSQQAAKSFYDRISFLIGSDSTHVIPGES-----PFNKSLASVIRGQ |
| NOV40k | PDPQDIISQSLQSPSQQAAKSFYDRISFLIGSDSTHVIPGES-----PFNKSLASVIRGQ |
| NOV40l | PDPQDIISQSLQSPSQQAAKSFYDRISFLIGSDSTHVIPGES-----PFNKSLASVIRGQ |
| NOV40m | PDPQDIISQSLQSPSQQAAKSFYDRISFLIGSDSTHVIPGES-----PFNKSLASVIRGQ |
| NOV40n | PDPQDIISQSLQSPSQQAAKSFYDRISFLIGSDSTHVIPGES-----PFNKSLASVIRGQ |
| NOV40o | PDPQDIISQSLQSPSQQAAKSFYDRISFLIGSDSTHVIPGES-----PFNKSLASVIRGQ |
| NOV40p | PDPQDIISQSLQSPSQQAAKSFYDRISFLIGSDSTHVIPGES-----PFNKSLASVIRGQ |
| NOV40q | WGGSNCEILKThCPDQCSGHGTYLQESGSCTCDPNWTGPDCSNEICSVDCGSHGVCMGGT |
| NOV40r | WGGSNCEILKTMCPDQCSGHGTYLQESGSCTCDPNWTGPDCSNEICSVDCGSHGVCMGGT |
| NOV40s | GQNFTLRQLGVCEPATRRGLAFCAEMGLPHRGYSISAGSDADTENEAVMSPEHAMRLWGR |
| NOV40t | WGGSNCEILKTMCPDQCSGHGTYLQESGSCTCDPNWTGPDCSNEICSVDCGSHGVCMGGT |
| NOV40u | PDPQDIISQSLQSPSQQAAKSFYDRISFLIGSDSTHVIPGES-----PFNKSLASVIRGQ |
| NOV40v | PDPQDIISQSLQSPSQQAAKSFYDRISFLIGSDSTHVIPGES-----PFNKSLASVIRGQ |
| NOV40w | -GKDTHYFIKTTTPESDLGTLRLTSGRKALENGINVTVSQSTTVVNGRTRRFADVEMQFG |
| NOV40x | WGGSNCEILKTMCPDQCSGHGTYLQESGSCTCDPNWTGPDCSNEICSVDCGSHGVCMGGT |
| NOV40y | WGGSNCEILKTMCPDQCSGHGTYLQESGSCTCDPNWTGPDCSNEICSVDCGSHGVCMGGT |
| NOV40z | WGGSNCEILKTMCPDQCSGHGTYLQESGSCTCDPNWTGPDCSNEICSVDCGSHGVCMGGT |
| NOV40aa | WGGSNCEILKTMCPDQCSGHGTYLQESGSCTCDPNWTGPDCSNEICSVDCGSHGVCMGGT |
| NOV40ab | GQNFTLRQLGVCEPATRRGLAFCAEMGLPHRGYSISAGSDADTENEAVMSPEHAMRLWGR |
| NOV40ac | GONFTLRQLGVCEPATRRGLAFCAEMGLPHRGYSISAGSDADTENEAVMSPEHAMRLWGR |
| NOV40a | CRCEEGWTGPACNORACHPRCAEHGTCKDGKCECSQGWNGEHCTIAHYLDKIVKDKIGYK |
| NOV40b | CRCEEGWTGPACNQRACHPRCAEHGTCKDGKCECSQGWNGEHCTI--------------- |
| NOV40c | CRCEEGWTGPACNQRACHPRCAEHGTCKDGKCECSQGWNGEHCTI--------------- |
| NOV40d | CRCEEGWTGPACNQRACHPRCAEHGTCKDGKCECSQGWNGEHCTIAHYLDKIVKDKIGYK |
| NOV40e | CRCEEGWTGPACNQRACHPRCAEHGTCKDGKCECSQGWNGEHCTIAHYLDKIVK------ |
| NOV40f | CRCEEGWTGPACNQRACHPRCAEHGTCKDGKCECSQGWNGEHCTIAHYLDKIVKDKIGYK |
| NOV40g | ALALHVRYGMTLDEEKARILEQA------------QQRVRDG------------------ |
| NOV40h | ESVQLQDSWVLG-SNVPLESRHFLFKTGTGTTP--------------------------- |
| NOV40i | CRCEEGWTGPACNQRACHPRCAEHGTCKDGKCECSQGWNGEHCTIAHYLDKIVKDKIGYK |
| NOV40j | VLTADGTPLIGVNVSFFHYPEYGYTITRQ------------------------------- |
| NOV40k | VLTADGTPLIGVNVSFFHYPEYGYTITRQ------------------------------- |
| NOV40l | VLTADGTPLIGVNVSFFHYPEYGYTITRQ------------------------------- |
| NOV40m | VLTADGTPLIGVNVSFFHYPEYGYTITRQ------------------------------- |
| NOV40n | VLTADGTPLIGVNVSFFHYPEYGYTITRQ------------------------------- |
| NOV40o | VLTADGTPLIGVNVSFFHYPEYGYTITRQ------------------------------- |
| NOV40p | VLTADGTPLIGVNVSFFHYPEYGYTITRQ------------------------------- |
| NOV40q | CRCEEGWTGPACNQRACHPRCAEHGTCKDGKCECSQGWNGEHCTIAHYLDKIVK------ |
| NOV40r | CRCEEGWTGPACNQRACHPRCAEHGTCKDGKCECSQGWNGEHCTIAHYLKIVK------- |
| NOV40s | GVKSGRSSCLSSRSNSALTLTDTEHENKSDSENEQPASNQGQSTLQPLPPSHKQHSAQHH |
| NOV40t | CRCEEGWTGPACNQRACHPRCAEHGTCKDGKCECSQGWNGEHCTIAHYLDKIVK------ |
| NOV40u | VLTADGTPLIGVNVSFFHYPEYGYTITRQ------------------------------- |
| NOV40v | VLTADGTPLIGVNVSFFHYPEYGYTITRQ------------------------------- |
| NOV40w | ALALHVRYGMTLDEEKARILEQARQRALARAWAREQQRVRDGEEGARLWTEGEKRQL--- |
| NOV40x | CRCEEGWTGPACNQRACHPRCAEHGTCKDGKCECSQGWNGEHCTIAHYLDKIVKDKIGYK |
| NOV40y | CRCEEGWTGPACNQRACHPRCAEHGTCKDGKCECSQGWNGEHCTIAHYLDKIVK------ |
| NOV40z | CRCEEGWTGPACNQRACHPRCAEHGTCKDGKCECSQGWNGEHCTIAHYLDKIVKDKIGYK |
| NOV40aa | CRCEEGWTGPACNQRACHPRCABHGTCKDGKCECSQGWNGEHCTIAHYLDKIVK------ |
| NOV40ab | GFKSGRSSCLSSRSNSALTLTDTEHENKSDSEN--------------------------- |
| NOV40ac | GVKSGRSSCLSSRSNSALTLTDTEHENKSDSEN--------------------------- |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | PSITSLNRNSLTNRRNQSPAPPAALPAELQTTPESVQLQDSWVLGSNVPLESRHFLFKTG |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | --EGCPGLCNSNGRCTLDQNGGHCVCQPGWRGAGCDVAMETLCTDSKDNEGDGLIDCMDP |
| NOV40b | --EGCPGLCNSNGRCTLDQNGWHCVCQPGWRGAGCD------------------------ |
| NOV40c | --EGCPGLSNSNGRCTLDQNGWHCVCQPGWRGAGCDVAMETLCTDSKDNEGDGLIDCMDP |
| NOV40d | --EGCPGLCNSNGRCTLDQNGWHCACQPGWRGAGCDVAMETLCTDSKDNEGDGLIDCMDP |
| NOV40e | --EGCPGLCNSNGRCTLDQNGWHCVCQPGWRGAGCDVAMETLCTDSKDNEGDGLIDCMDP |
| NOV40f | --EGCPGLCNSNGRCTLDQNGWHCACQPGWRGAGCDVAMETLCTDSKDNEGDGLIDCMDP |
| NOV40g | ------KVQGYDGYYVLSVEQYPELADSANNIQFLRQSEIGRRVDG-------------- |
| NOV40h | -----LFSTATPGYTMASGSVYSPPTRPLPRNTLSRSAFKFKKS-SKYCSWKCTALCAVG |
| NOV40i | --EGCPGLCNSNGRCTLDQNGGHCVCQPGWRGAGCDVAMETLCTDSKDNEGDGLIDCMDP |
| NOV40j | -----------DGMFDLVANGGASLTLVFERSP--------------------------- |
| NOV40k | -----------DGMFDLVASGGASLTLVFERSP--------------------------- |
| NOV40l | -----------DGMFDLVANGGASLTLVFERSP--------------------------- |
| NOV40m | -----------DGMFDLVANGGASLTLVFERSP--------------------------- |
| NOV40n | -----------DGMFDLVANGGASLTLVFERSP--------------------------- |
| NOV40o | -----------DGMFDLVANGGASLTLVFERSP--------------------------- |
| NOV40p | -----------DGMFDLVANGGASLTLVFERSP--------------------------- |
| NOV40q | --EGCPGLCNSNGRCTLDQNGWHCVCQPGWRGAGCDVAMETLCTDSKDNEGDGLIDCMDP |
| NOV40r | --EGCPGLCNSNGRCTLDQNGWHCVCQPGWRGAGCDVAMETLCTDSKDNEGDGLIDCMDP |
| NOV40s | TGTTPLFSTATPGYTMASGSVYSPPTRPLPRNTLSRSAFKFKKS-SKYCSWKCTALCAVG |
| NOV40t | --EGCPGLCNSNGRCTLDQNGWHCVCQPGWRGAGCD------------------------ |
| NOV40u | -----------DGMFDLVANGGASLTLVFERSP--------------------------- |
| NOV40v | -----------DGMFDLVANGGASLTLVFERSP--------------------------- |
| NOV40w | --LSAGKQGYDGYYVLSVEQYPELADSANNIQFLRQSEIGRR------------------ |
| NOV40x | --EGCPGLCNSNGRCTLDQNGWHCACQPGWRGAGCDVAMETLCTDSKDNEGDGLIDCMDP |
| NOV40y | --EGCPGLCNSNGRCTLDQNGWHCVCQPGWRGAGCDVAMETLCTDSKDNEGDGLIDCMDP |
| NOV40z | --EGCPGLCNSNGRCTLDQNGWHCACQPGWRGAGCDVAMETLCTDSKDNEGDGLIDCMDP |
| NOV40aa | --EGCPGLCNSNGRCTLDQNGWHCVCQPGWRGAGCDVANETLCTDSKDNEGDGLIDCMDP |
| NOV40ab | -----GGSSSWFGFHWNFYVSKASCLLRLPRIFLSHNYNVNKEMREKLC----------- |
| NOV40ac | -----GGSSSWFGFHWNFYVGKASCLLRLPRIFLSHNYNVNKEMREKLC----------- |
| NOV40a | DCCLQSSCQNQPYCRGLPDPQDIISOSLQSPSQQAAKSFYDRISFLIGSDSTHVIPGESP |
| NOV40b | ------------------------------------------------------------ |
| NOV40c | DCCLQSSCQNQPYCRGLPDPQDIISQSLQSPSQQAAKSFYDRISFLIGSDSTHVIPGESP |
| NOV40d | DCCLQSSCQNQPYCRGLPDPQGIISQSLQSPSQQAAKSFYDRISFLIGSDSTHVIPGESP |
| NOV40e | DCCLQSSCQNQPYCRGLPDPODIISQSLQSPSQQAAKSFYDRISFLIGSDSTHVIPGESP |
| NOV40f | DCCLQSSCQNQPYCRGLPDPQGIISQSLQSPSQQAAKSFYDRISFLIGSDSTHVIPGESP |
| NOV40g | ------------------------------------------------------------ |
| NOV40h | VSVLLAILLSYFIAMHLFGLNWQLQQTENDTFENGKVNSDTMPTNTVSLPSGDNGKLGGF |
| NOV40i | DCCLQSSCQNQPYCRGLPDPQDIISQSLQSPSQQAAKSFYDRISFLIGSDSTHVIPGESP |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | DCCLQSSCQNQPYCRGLPDPQDIISOSLQSPSQQAAKSFYDRISFLIGSDSTHVIPGESP |
| NOV40r | DCCLQSSCQNQPYCRGLPDPQDIISQSLQSPSQQAAKSFYDRISFLIGSDSTHVIPGESP |
| NOV40s | VSVLLAILLSYFIAMHLFGLNWQLQQTENDTFENGKVNSDTMPTNTVSLPSGDNGKLGGF |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | DCCLQSSCQNQPYCRGLPDPQGIISQSLQSPSQQAAKSFYDRISFLIGSDSTHVIPGESP |
| NOV40y | DCCLQSSCQNQPYCRGLPDPQDIISQSLQSPSQQAAKSFYDRISFLIGSDSTHVIPGESP |
| NOV40z | DCCLQSSCQNQPYCRGLPDPQGIISQSLQSPSQQAAKSFYDRISFLIGSDSTHVIPGESP |
| NOV40aa | DCCLQSSCQNQPYCRGLPDPQDIISQSLQSPSQOAAKSFYDRISFLIGSDSTHVIPGESP |
| NOV40ab | ------------------------------------------------------------ |
| NOV40ac | ------------------------------------------------------------ |
| NOV40a | FNKSLASVIRGQVLTADGTPLIGVNVSFFHYPEYGYTITRQDGMFDLVANGGASLTLVFE |
| NOV40b | ------------------------------------------------------------ |
| NOV40c | FNKSLASVIRGQVLTADGTPLIGVNVSFFHYPEYGYTITRQDGMFDLVANGGASLTLVFE |
| NOV40d | FNKSLASVIRGQVLTADGTPLIGVNVSFFHYPEYGYTITRQDGMFDLVANGGASLTLVFE |
| NOV40e | FNKSLASVIRGQVLTADGTPLIGVNVSFFHYPEYGYTITRQDGMFDLVANGGASLTLVFE |
| NOV40f | FNKSLASVIRGQVLTADGTPLIGVNVSFFHYPEYGYTITRQDGMFDLVANGGASLTLVFE |
| NOV40g | ------------------------------------------------------------ |
| NOV40h | TQENNTIDSGELDIGRRAIQEIPPGIFWRSQLFIDQPQFLKFNISLQKDALIGVYGRKGL |
| NOV40i | FNKSLASVIRGQVLTADGTPLIGVNVSFFHYPEYGYTITRQDGMFDLVANGGASLTLVFE |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | FNKSLASVIRGQVLTADGTPLIGVNVSFFHYPEYGYTITRQDGMFDLVANGGASLTLVFE |
| NOV40r | FNKSLASVIRGQVLTADGTPLIGVNVSFFHYPEYGYTITRQDGMFDLVANGGASLTLVFE |
| NOV40s | TQENNTIDSGELDIGRRAIQEIPPGIFWRSQLFIDQPQFLKFNISLQKDALIGVYGRKGL |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | FNKSLASVIRGQVLTADGTPLIGVNVSFFHYPEYGYTITRQDGMFDLVANGGASLTLVFE |
| NOV40y | FNKSLASVIRGQVLTADGTPLIGVNVSFFHYPEYGYTITRQDGMFDLVANGGASLTLVFE |
| NOV40z | FNKSLASVIRGQVLTADGTPLIGVNVSFFHYPEYGYTITRQDGMFDLVANGGASLTLVFE |
| NOV40aa | FNKSLASVIRGQVLTADGTPLIGVNVSFFHYPEYGYTITRQDGMFDLVANGGASLTLVFE |
| NOV40ab | ------------------------------------------------------------ |
| NOV40ac | ------------------------------------------------------------ |
| NOV40a | RSPFLTQYHTVWIPWNVFYVMDTLVMEKEENDIPSCDLSGFVRPNPIIVSSPLSTFFRSS |
| NOV40b | ------------------------------------------------------------ |
| NOV40c | RSPFLTQYHTVWIPWNVFYVMDTLVMKKEENDIPSCDLSGFVRPNPIIVSSPLSTFFRSS |
| NOV40d | RSPFLTQYHTVWIPWNVFYVMDTLVMKKEENDIPSCDLSGFVRPHPIIVSSPLSTFFRSS |
| NOV40e | RSPFLTQYHTVWIPWNVFYVMDTLVMKKEENDIPSCDLSGFVRPNPIIVSSPLSTFFRSS |
| NOV40f | RSPFLTQYHTVWIPWNVFYVMDTLVMKKEENDIPSCDLSGFVRPNPIIVSSPLSTFFRSS |
| NOV40g | ------------------------------------------------------------ |
| NOV40h | PPSHTQYDFVELLDGSRLIALEG------------------------------------- |
| NOV40i | RSPFLTQYHTVWIPWNVFYVMDTLVMEKEENDIPSCDLSGFVRPNPIIVSSPLSTFFRSS |
| NOV40j | ---FLTQYHTVWIPWNVFYVMDTLVMKKEENDIPSCDLSGFVRPNPIIVSSPLSTFFRSS |
| NOV40k | ---FLTQYHTVWIPWNVFYVMDTLVMKKEENDIPSCDLSGFVRPNPIIVSSPLSTFFRSS |
| NOV40l | ---FLTQYHTVWIPWNVFYVMDTLVMKKEENDIPSCDLSGFVRPNPIIVSSPLSTFFRSS |
| NOV40m | ---FLTQYHTVWIPWNVFYVMDTLVMKKEENDIPSCDLSGFVRPNPIIVSSPLSTFFRSS |
| NOV40n | ---FLTQYHTVWIPWNVFYVMDTLVMKKEENDIPSCDLSGFVRPNPIIVSSPLSTFFRSS |
| NOV40o | ---FLTQYHTVWIPWNVFYVMDTLVMKKEENDIPSCDLSGFVRPNPIIVSSPLSTFFRSS |
| NOV40p | ---FLTQYHTVWIPWNVFYVMDTLVMKKEENDIPSCDLSGFVRPNPIIVSSPLSTFFRSS |
| NOV40q | RSPFLTQYHTVWIPWNVFYVMDTLVMKKEENDIPSCDLSGFVRPNPIIVSSPLSTFFRSS |
| NOV40r | RSPFLTQYHTVWIPWNVFYVMDTLVMKKEENDIPSCDLSGFVRPNPIIVSSPLSTFFRSS |
| NOV40s | PPSHTQYDFVELLDGSRLIAREQRSLLETERAGRQARSVSLHEAGFIQYLDSGIWHLAFY |
| NOV40t | ------------------------------------------------------------ |
| NOV40u | ---FLTQYHTVWIPWNVFYVMDTLVMBKEENDIPSCDLSGFVRPNPIIVSSPLSTFFRSS |
| NOV40v | ---FLTQYHTVWIPWNVFYVNDTLVMKKEENDIPSCDLSGFVRPNPIIVSSPLSTFFRSS |
| NOV40w | ------------------------------------------------------------ |
| NOV40x | RSPFLTQYHTVWIPWNVFYVMDTLVMKKEENDIPSCDLSGFVRPNPIIVSSPLSTFFRSS |
| NOV40y | RSPFLTQYHTVWIPWNVFYVMDTLVMKKEENDIPSCDLSGFVRPNPIIVSSPLSTFFRSS |
| NOV40z | RSPFLTQYHTVWIPWNVFYVMDTLVMKKEENDIPSCDLSGFVRPNPIIVSSPLSTFFRSS |
| NOV40aa | RSPFLTQYHTVWIPWNVFYVMDTLVMKKEENDIPSCDLSGFVRPNPIIVSSPLSTFFRSS |
| NOV40ab | ------------------------------------------------------------ |
| NOV40ac | ------------------------------------------------------------ |
| NOV40a | PEDSPIIPETQVLHEETTIPGTDLKLSYLSSRAAGYKSVLKITMTQSIIPFNLMKVHLNV |
| NOV40b | ------------------------------------------------------------ |
| NOV40c | PEDSPIIPETQVLHEETTIPGTDLKLSYLSSRAAGYKSVLKITMTQSIIPFNLMKVHLMV |
| NOV40d | PEDSPIIPETQVLHEETTIPGTDLKLSYLSSRAAGYKSVLKITMTQSIIPFNLMKVHLMV |
| NOV40e | PEDSPIIPETQVLHEETTIPGTDLKLSYLSSRAAGYKSVLKITMTQSIIPFNLMKVHLMV |
| NOV40f | PEDSPIIPETQVLHEETTIPGTDLKLSYLSSRAAGYKSVLKITMTQSIIPFNLMKVHLMV |
| NOV40g | ------------------------------------------------------------ |
| NOV40h | ------------------------------------------------------------ |
| NOV40i | PEDSPIIPETQVLHEETTIPGTDLKLSYLSSRAAGYKSVLKITMTQSIIPFNLMKVHLMV |
| NOV40j | PEDSPIIPETQVLHEETTIPGTDLKLSYLSSRAAGYKSVLKITMTQSIIPFNLMKVHLMV |
| NOV40k | PEDSPIIPETQVLHEETTIPGTDLKLSYLSSRAAGYKSVLKITMTQSIIPFNLMKVHLMV |
| NOV40l | PEDSPIIPETQVLHEETTIPGTDLKLSYLSSRAAGYKSVLKITMTQSIIPFNLMKVHLMV |
| NOV40m | PEDSPIIPETQVLHEETTIPGTDLKLSYLSSRAAGYKSVLKITMTQSIIPFNLMKVHLMV |
| NOV40n | PEDSPIIPETQVLHEETTIPGTDLKLSYLSSRAAGYKSVLKITMTQSIIPFNLMKVHLMV |
| NOV40o | PEDSPIIPETQVLHEETTIPGTDLKLSYLSSRAAGYKSVLKITMTQSIIPFNLMKVHLMV |
| NOV40p | PEDSPIIPETQVLHEETTIPGTDLKLSYLSSRAAGYKSVLKITMTQSIIPFNLMKVHLMV |
| NOV40q | PEDSPIIPETQVLHEETTIPGTDLKLSYLSSRAAGYKSVLKITMTQSIIPFNLMKVHLMV |
| NOV40r | PEDSPIIPETQVLHEETTIPGTDLKLSYLSSRAAGYKSVLKITMTQSIIPFNLMKVHLMV |
| NOV40s | NDGKNAEQVSFNTIVIESVVECPRNCHGNGECVSGTCHCFPGFLGPDCSRAACPVLCSGN |
| NOV40t | ------------------------------------------------------------ |
| NOV40u | PEDSPIIPETQVLHEETTIPGTDLKLSYLSSRAAGYKSVLKITMTQSIIPFNLMKVHLMV |
| NOV40v | PEDSPIIPETQVLHEETTIPGTDLKLSYLSSRAAGYKSVLKITMTQSIIPFNLMKVHLMV |
| NOV40w | ------------------------------------------------------------ |
| NOV40x | PEDSPIIPETQVLHEETTIPGTDLKLSYLSSRAAGYKSVLKITMTQSIIPFNLMKVHLMV |
| NOV40y | PEDSPIIPETQVLHEETTIPGTDLKLSYLSSRAAGYKSVLKITMTQSIIPFNLMKVHLMV |
| NOV40z | PEDSPIIPETQVLHEETTIPGTDLKLSYLSSRAAGYKSVLKITMTQSIIPFNLMKVHLMV |
| NOV40aa | PEDSPIIPETQVLHEETTIPGTDLKLSYLSSRAAGYKSVLKITMTQSIIPFNLMKVHLMV |
| NOV40ab | ------------------------------------------------------------ |
| NOV40ac | ------------------------------------------------------------ |
| NOV40a | AVVGRLFQKWFPASPNLAYTFIWDKTDAYNQKVYGLSEAVVSVGYEYESCLDLTLWEKRT |
| NOV40b | ------------------------------------------------------------ |
| NOV40c | AVVGRLFQKWFPASPNLAYTFIWDKTDAYNQKVYGLSEAVVSVGYEYESCLDLTLWEKRT |
| NOV40d | AVVGRLFQKWFPASPNLAYTFIWDKTDAYNQKVYGLSEAVVSVGYEYESCLDLTLWEKRT |
| NOV40e | AVVGRLFQKWFPASPNLAYTFIWDKTDAYNQKVYGLSEAVVSVGYEYESCLDLTLWEKRT |
| NOV40f | AVVGRLFQKWFPASPNLAYTFIWDKTDAYNQKVYGLSEAVVSVGYEYESCLDLTLWEKRT |
| NOV40g | ------------------------------------------------------------ |
| NOV40h | ------------------------------------------------------------ |
| NOV40i | AVVGRLFQKWFPASPNLAYTFIWDKTDAYNQKVYGLSEAVVSVGYEYESCLDLTLWEKRT |
| NOV40j | AVVGRLFQKWFPASPNLAYTFIWDKTDAYNQKVYGLSEAVVSVGYEYESCLDLTLWEKRT |
| NOV40k | AVVGRLFQKWFPASPNLAYTFIWDKTDAYNQKVYGLSEAVVSVGYEYESCLDLTLWEKRT |
| NOV40l | AVVGRLFQKWFPASPNLAYTFIWDKTDAYNQKVYGLSEAVVSVGYEYESCLDLTLWEKRT |
| NOV40m | AVVGRLFQKWFPASPNLAYTFIWDKTDAYNQKVYGLSEAVVSVGYEYESCLDLTLWEKRT |
| NOV40n | AVVGRLFQKWFPASPNLAYTFIWDKTDAYNQKVYGLSEAVVSVGYEYESCLDLTLWEKRT |
| NOV40o | AVVGRLFQKWFPASPNLAYTFIWDKTDAYNQKVYGLSEAVVSVGYEYESCLDLTLWEKRT |
| NOV40p | AVVGRLFQKWFPASPNLAYTFIWDKTDAYNQKVYGLSEAVVSVGYEYESCLDLTLWEKRT |
| NOV40q | AVVGRLFQKWFPASPNLAYTFIWDKTDAYNQKVYGLSEAVVSVGYEYESCLDLTLWEKRT |
| NOV40r | AVVGRLFQKWFPASPNLAYTFIWDKTDAYNQKVYGLSEAVVSVGYEYESCLDLTLWEKRT |
| NOV40s | GQYSKGRCLCFSGWKGTECDVPTWQCIDPQCGGRGICIMGSCACSSGYKGESCEEADCID |
| NOV40t | ------------------------------------------------------------ |
| NOV40u | AVVGRLFQKWFPASPNLAYTFIWDKTDAYNQKVYGLSEAVVSVGYEYESCLDLTLWEKRT |
| NOV40v | AVVGRLFQKWFPASPNLAYTFIWDKTDAYNQKVYGLSEAVVSVGYEYESCLDLTLWEKRT |
| NOV40w | ------------------------------------------------------------ |
| NOV40x | AVVGRLFQKWFPASPNLAYTFIWDKTDAYNQKVYGLSEAVVSVGYEYESCLDLTLWEKRT |
| NOV40y | AVVGRLFQKWFPASPNLAYTFIWDKTDAYNQKVYGLSEAVVSVGYEYESCLDLTLWEKRT |
| NOV40z | AVVGRLFQKWFPASPNLAYTFIWDKTDAYNQKVYGLSEAVVSVGYEYESCLDLTLWEKRT |
| NOV40aa | AVVGRLFQKWFPASPNLAYTFIWDKTDAYNQKVYGLSEAVVSVGYEYESCLDLTLWEKRT |
| NOV40ab | ------------------------------------------------------------ |
| NOV40ac | ------------------------------------------------------------ |
| NOV40a | AILQGYELDASNMGGWTLDKHHVLDVQNGILYKGNGENQFISQQPPVVSSIMGNGRRRSI |
| NOV40b | ------------------------------------------------------------ |
| NOV40c | AILQGYELDASNMGGWTLDKHHVLDVQNGILYKGNGENQFISQQPPVVSSVD-------- |
| NOV40d | AILQGYELDASNMGGWTLDKHHVLDVQNGILYKGNGENQFISQQPPVVSSVDPAASSRE- |
| NOV40e | AILQGYELDASNMGGWTLDKHHVLDVQNGILYKGNGENQFISQQPPVVSSVDATHDWRLL |
| NOV40f | AILQGYELDASNMGGWTLDKHHVLDVQNGILYKGNGENQFISQQPPVVSSLEG------- |
| NOV40g | ------------------------------------------------------------ |
| NOV40h | ------------------------------------------------------------ |
| NOV40i | AILQGYELDASNMGGWTLDKHHVLDVQNGILYKGNGENQFISQQPPVVSSIMGNGRRRSI |
| NOV40j | AILQGYELDASNMGGWTLDKHHVLDVQNGILYKGNGENQFISQQPPVVSSIMGNGRRRSI |
| NOV40k | AILQGYELDASNMGGWTLDKHHALDVQNGILYKGNGENQFISQQPPVVSSIMGNGRRRSI |
| NOV40l | AILQGYELDASNMGGWTLDKHHALDVQNGILYKGNGENQFISQQPPVVSSIMGNGRRRSI |
| NOV40m | AILQGYELDASNNGGWTLDKHHVLDVQNGILYKGNGENQFISQQPPVVSSIMGNGRRRSI |
| NOV40n | AILQGYELDASNMGGWTLDKHHVLDVQNGILYKGNGENQFISQQPPVVSSIMGNGRRRSI |
| NOV40o | AILQGYELDASNMGGWTLDKHHVLDVQNGILYKGNGENQFISQQPPVVSSIMGNGRRRSI |
| NOV40p | AILQGYELDASNMGGWTLDKHHVLDVQNGILYKGNGENQFISQQPPVVSSIMGNGRRRSI |
| NOV40q | AILQGYELDASNMGGWTLDKHHVLDVQNGILYKGNGENQFISQQPPVVSS---------- |
| NOV40r | AILQGYELDASNMGGWTLDKHHVLDVQNGILYKGNGENQFISQQPPVVSSLEG------- |
| NOV40s | PGCSNHGVCIHGECHCSPGWGGSNCEILKTMCPDQCSGHGTYLQESGSCTCDPNWTGPDC |
| NOV40t | ------------------------------------------------------------ |
| NOV40u | AILQGYELDASNMGGWTLDKHHVLDVQNGILYKGNGENQFISQQPPVVSSIMGNGRRRSI |
| NOV40v | AILQGYELDASNMGGWTLDKHHVLDVQNGILYKGNGENQFISQQPPVVSSIMGNGRRRSI |
| NOV40w | ------------------------------------------------------------ |
| NOV40x | AILQGYELDASNMGGWTLDKHHVLDVQNGILYKGNGENQFISQQPPVVSS---------- |
| NOV40y | AILQGYELDASNMGGWTLDKHHVLDVQNGILYKGNGENQFISQQPPVVSS---------- |
| NOV40z | AILQGYELDASNMGGWTLDKHHVLDVQNGILYKGNGENQFISQQPPVVSS---------- |
| NOV40aa | AILQGYELDASNMGGWTLDKHHVLDVQNGILYKGNGENQFISQQPPVVSS---------- |
| NOV40ab | ------------------------------------------------------------ |
| NOV40ac | ------------------------------------------------------------ |
| NOV40a | SCPSCNGQADGNKLLAPVALACGIDGSLYVGDFNYVRRIFPSGNVTSVLELRNKDFRHSS |
| NOV40b | ------------------------------------------------------------ |
| NOV40c | ------------------------------------------------------------ |
| NOV40d | ------------------------------------------------------------ |
| NOV40e | VIFSSAVAASWY------------------------------------------------ |
| NOV40f | ------------------------------------------------------------ |
| NOV40g | ------------------------------------------------------------ |
| NOV40h | ------------------------------------------------------------ |
| NOV40i | SCPSCNGQADGNKLLAPVALACGIDGSLYVGDFNYVRRIFPSGNVTSVLELRNKDFRHSS |
| NOV40j | SCPSCNGQADGNKLLAPVALACGIDGSLYVGDFNYVRRIFPSGNVTSVLELRNKDFRHSS |
| NOV40k | SCPSCNGQADGNKLLAPVALACGIDGSLYVGDFNYVRRIFPSGNVTSVLELRNKDFRHSS |
| NOV40l | SCPSCNGQADGNKLLAPVALACGIDGSLYVGDFNYVRRIFPSGNVTSVLELS-------S |
| NOV40m | SCPSCNGQADGNKLLAPVALACGIDGSLYVGDFNYVRRIFPSGNVTSVLELS-------S |
| NOV40n | SCPSCNGQADGNKLLAPVALACGIDGSLYVGDFNYVRRIFPSGNVTSVLELS-------S |
| NOV40o | SCPSCNGQADGNKLLAPVALACGIDGSLYVGDFNYVRRIFPSGNVTSVLELS-------S |
| NOV40p | SCPSCNGQADGNKLLAPVALACGIDGSLHVGDFNYVRRIFPSGNVTSVLELS-------S |
| NOV40q | ------------------------------------------------------------ |
| NOV40r | ------------------------------------------------------------ |
| NOV40s | SNEICSVDCGSHGVCMGGTCRCEEGWTGPACNQRACHPRCAEHGTCKDGKCECSQGWNGE |
| NOV40t | ------------------------------------------------------------ |
| NOV40u | SCPSCNGQADGNKLLAPVALACGIDGSLYVGDFNYVRRIFPSGNVTSVLELRNKDFRHSS |
| NOV40v | SCPSCNGQADGNKLLAPVALACGIDGSLYVGDFNYVRRIFPSGNVTSVLELS-------S |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | NPAHRYYLATDPVTGDLYVSDTNTRRIYRPKSLTGAKDLTKNAEVVAGTGEQCLPFDEAR |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | NPAHRYYLATDPVTGDLYVSDTNTRRIYRPKSLTGAKDLTKNAEVVAGTGEQCLPFDEAR |
| NOV40j | NPAHRYYLATDPVTGDLYVSDTNTRRIYRPKSLTGAKDLTKNAEVVAGTGEQCLPFDEAR |
| NOV40k | NPAHRYYLATDPVTGDLYVSDTNTRRIYRPKSLTGAKDLTKNAEVVAGTGEQCLPFDEAR |
| NOV40l | NPAHRYYLATDPVTGDLYVSDTNTRRIYRPKSLTGAKDLTKNAEVVAGTGEQCLPFDEAR |
| NOV40m | NPAHRYYLATDPVTGDLYVSDTNTRRIYRPKSLTGAKDLTKNAEVVAGTGEQCLPFDEAR |
| NOV40n | NPAHRYYLATDPVTGDLYVSDTNTRRIYRPKSLTGAKDLTKNAEVVAGTGEQCLPFDEAR |
| NOV40o | NPAHRYYLATDPVTGDLYVSDTNTRRIYRPKSLTGAKDLTKNAEVVAGTGEQCLPFDEAR |
| NOV40p | NPAHRYYLATDPVTGDLYVSDTNTRRIYRPKSLTGAKDLTKNAEVVAGTGEQCLPFDEAR |
| NOV40q | ------------------------------------------------------------ |
| NOV40r | ------------------------------------------------------------ |
| NOV40s | HCTIAHYLDKIVKDKIGYKEGCPGLCNSNGRCTLDQNGGHCVCQPGWRGAGCDVAMETLC |
| NOV40t | ------------------------------------------------------------ |
| NOV40u | NPAHRYYLATDPVTGDLYVSDTNTRRIYRPKSLTGAKDLTKNAEVVAGTGEQCLPFDEAR |
| NOV40v | NPAHRYYIATDPVTGDLYVSDTNTRRIYRPKSLTGAKDLTKNAEVVAGTGEQCLPFDEAR |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | CGDGGKAVEATLMSPKGMAVDKNGLIYFVDGTMIRKVDQNGIISTLLGSNDLTSARPLTC |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | CGDGGKAVEATLMSPKGMAVDKNGLIYFVDGTMIRKVDQNGIISTLLGSNDLTSARPLTC |
| NOV40j | CGDGGKAVEATLMSPKGMAVDKNGLIYFVDGTMIRKVDQNGIISTLLGSNDLTSARPLTC |
| NOV40k | CGDGGKAVEATLMSPKGMAVDKNGLIYFVDGTMIRKVDQNGIISTLLGSNDLTSARPLTC |
| NOV40l | CGDGGKAVEATLMSPKGMAVDKNGLIYFVDGTMIRKVDQNGIISTLLGSNDLTSARPLTC |
| NOV40m | CGDGGKAVEATLMSPKGMAVDKNGLIYFVDGTMIRKVDQNGIISTLLGSNDLTSARPLTC |
| NOV40n | CGDGGKAVEATLMSPKGMAVDKNGLIYFVDGTMIRKVDQNGIISTLLGSNDLTSARPLTC |
| NOV40o | CGDGGKAAEATLMSPKGMAVDKNGLIYFVDGTMIRKVDQNGIISTLLGSNDLTSARPLTC |
| NOV40p | CGDGGKAVEATLMSPKGMAVDKNGLIYFVDGTMIRKVDQNGIISTLLGSNDLTSARPLTC |
| NOV40q | ------------------------------------------------------------ |
| NOV40r | ------------------------------------------------------------ |
| NOV40s | TDSKDNEGDGLIDCMDPDCCLQSSCQNQPYCRGLPDPQDIISQSLQSPSQQAAKSFYDRI |
| NOV40t | ------------------------------------------------------------ |
| NOV40u | CGDGGKAVEATLMSPKGMAVDKNGLIYFVDGTMIRKVDQNGIISTLLGSNDLTSARPLTC |
| NOV40v | CGDGGKAVEATLMSPKGMAVDKNGLIYFVDGTMIRKVDQNGIISTLLGSNDLTSARPLTC |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | DTSMHISQVRLEWPTDLAINPMDNSIYYLDNNVVLQITENRQVRIAAGRPMHCQVPGVEY |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | DTSMHISQVRLEWPTDLAINPMDNSIYYLDNNVVLQITENRQVRIAAGRPMHCQVPGVEY |
| NOV40j | DTSMHISQVRLEWPTDLAINPMDNSIYVLDNVD--------------------------- |
| NOV40k | DTSMHISQVRLEWPTDLAINPMDNSIYVLDNVD--------------------------- |
| NOV40l | DTSMHISQVRLEWPTDLAINPMDNSIYVLDNVD--------------------------- |
| NOV40m | DTSMHISQVRLEWPTDLAINPMDNSIYVLDNVD--------------------------- |
| NOV40n | DTSMHISQVRLEWPTDLAINPMDNSIYVLDNVD--------------------------- |
| NOV40o | DTSMHISQVRLEWPTDLAINPMDNSIYVLDNVD--------------------------- |
| NOV40p | DTSMHISQVRLEWPTDLAINPMDNSIYVLDNVD--------------------------- |
| NOV40q | ------------------------------------------------------------ |
| NOV40r | ------------------------------------------------------------ |
| NOV40s | SFLIGSDSTHVIPGESPFNKSLASVIRGQVLTADGTPLIGVNVSFFHYPEYGYTITRQDG |
| NOV40t | ------------------------------------------------------------ |
| NOV40u | DTSMHISQVRLEWPTDLAINPMDNSIYYLDNNVVLQITENRQVRIAAGRPMHCQVPGVEY |
| NOV40v | DTSMHISQVRLEWPTDLAINPMDNSIYVLDN----------------------------- |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | PVGKHAVQTTLESATAIAVSYSGVLYITETDEKKINRIRQVTTDGEISLVAGIPSECDCK |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | PVGKHAVQTTLESATAIAVSYSGVLYITETDEKKINRIRQVTTDGEISLVAGIPSECDCK |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV4Qq | ------------------------------------------------------------ |
| NOV40r | ------------------------------------------------------------ |
| NOV40s | MFDLVANGGASLTLVFERSPFLTQYHTVWIPWNVFYVMDTLVMEKEENDIPSCDLSGFVR |
| NOV40t | ------------------------------------------------------------ |
| NOV40u | PVGKHAVQTTLESATAIAVSYSGVLYITETDEKKINRIRQVTTDGEISLVAGIPSECDCK |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | NDANCDCYQSGDGYAKDAKLSAPSSLAASPDGTLYIADLGNIRIRAVSKNKPLLNSMNFY |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | NDANCDCYQSGDGYAKDAKLSAPSSLAASPDGTLYIADLGNIRIRAVSKNKPLLNSMNFY |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | PNPIIVSSPLSTFFRSSPEDSPIIPETQVLHEETTIPGTDLKLSYLSSRAAGYKSVLKIT |
| NOV40t | ------------------------------------------------------------ |
| NOV40u | NDANCDCYQSGDGYAKDAKLSAPSSLAASPDGTLYIADLGNIRIRAVSKNKPLLNSMNFY |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | EVASPTDQELYIFDINGTHQYTVSLVTGDYLYNFSYSNDNDITAVTDSNGNTLRIRRDPN |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | EVASPTDQELYIFDINGTHQYTVSLVTGDYLYNFSYSNDNDITAVTDSNGNTLRIRRDPN |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | MTQSIIPFNLMKVHLMVAVVGRLFQKWFPASPNLAYTFIWDKTDAYNQKVYGLSEAVVSV |
| NOV40t | ------------------------------------------------------------ |
| NOV40u | EVASPTDQELYIFDINGTHQYTVSLVTGDYLYNFSYSNDNDITAVTDSNGNTLRIRRDPN |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | RMPVRVVSPDNQVIWLTIGTNGCLKGMTAQGLELVLFTYHGNSGLLATKSDETGWTTFFD |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | RMPVRVVSPDNQVIWLTIGTNGCLKGMTAQGLELVLFTYHGNSGLLATKSDETGWTTFFD |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | GYEYESCLDLTLWEKRTAILQGYELDASNMGGWTLDKHHVLDVQNGILYKGNGENQFISQ |
| NOV40t | ------------------------------------------------------------ |
| NOV40u | RMPVRVVSPDNQVIWLTIGTNGCLKGMTAQGLELVLFTYHGNSGLLATKSDETGWTTFFD |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | YDSEGRLTNVTFPTGVVTNLHGDMDKAITVDIESSSREEDVSITSNLSSIDSFYTMVQDQ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | YDSEGRLTNVTFPTGVVTNLHGDMDKAITVDIESSSREEDVSITSNLSSIDSFYTMVQDQ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | QPPVVSSIMGNGRRRSISCPSCNGQADGNKLLAPVALACGIDGSLYVGDFNYVRRIFPSG |
| NOV40t | ------------------------------------------------------------ |
| NOV40u | YDSEGRLTNVTFPTGVVTNLHGDMDKAITVDIESSSREEDVSITSNLSSIDSFYTMVQDQ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | LRNSYQIGYDGSLRIIYASGLDSHYQTEPHVLAGTANPTVAKRNMTLPGENGQNLVEWRF |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | LRNSYQIGYDGSLRIIYASGLDSHYQTEPHVLAGTANPTVAKRNMTLPGENGQNLVEWRF |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | NVTSVLELRNKDFRHSSNPAHRYYLATDPVTGDLYVSDTNTRRIYRPKSLTGAKDLTKNA |
| NOV40t | ------------------------------------------------------------ |
| NOV40u | LRNSYQIGYDGSLRIIYASGLDSHYQTEPHVLAGTANPTVAKRNMTLPGENGQNLVEWRF |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | RKEQAQGKVNVFGRKLRVNGRNLLSVDFDRTTKTEKIYDDHRKFLLRIAYDTSGHPTLWL |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | RKEQAQGKVNVFGRKLRVNGRNLLSVDFDRTTKTEKIYDDHRKFLLRIAYDTSGHPTLWL |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV400 | ------------------------------------------------------------ |
| NOV40p | ------------------------------------------------------------ |
| NOV40q | ------------------------------------------------------------ |
| NOV40r | ------------------------------------------------------------ |
| NOV40s | EVVAGTGEQCLPFDEARCGDGGKAVEATLMSPKGMAVDKNGLIYFVDGTMIRKVDQNGII |
| NOV40t | ------------------------------------------------------------ |
| NOV40u | RKEQAQGKVNVFGRKLRVNGRNLLSVDFDRTTKTEKIYDDHRKFLLRIAYDTSGHPTLWL |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | PSSKLMAVNVTYSSTGQIASIQRGTTSEKVDYDGQGRIVSRVFADGKTWSYTYLEKSMVL |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | PSSKLMAVNVTYSSTGQIASIQRGTTSEKVDYDGQGRIVSRVFADGKTWSYTYLEKSMVL |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | STLLGSNDLTSARPLTCDTSMHISQVRLEWPTDLAINPMDNSIYVLDNNVVLQITENRQV |
| NOV40t | ------------------------------------------------------------ |
| NOV40u | PSSKLMAVNVTYSSTGQIASIQRGTTSEKVDYDGQGRIVSRVFADGKTWSYTYLEKSMVL |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | LLHSQRQYIFEYDMWDRLSAITMPSVARHTMQTIRSIGYYRNIYNPPESNASIITDYNEE |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | LLHSQRQYIFEYDMWDRLSAITMPSVARHTNQTIRSIGYYRNIYNPPESNASIITDYNEE |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | RIAAGRPMHCQVPGVEYPVGKHAVQTTLESATAIAVSYSGVLYITETDEKKINRIRQVTT |
| NOV40t | ------------------------------------------------------------ |
| NOV40u | LLHSQRQYIFEYDMWDRLSAITMPSVARHTMQTIRSIGYYRNIYNPPESNASIITDYNEE |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | GLLLQTAFLGTSRRVLFKYRRQTRLSEILYDSTRVSFTYDETAGVLKTVNLQSDGFICTI |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | GLLLQTAFLGTSRRVLFKYRRQTRLSEILYDSTRVSFTYDETAGVLKTVNLQSDGFICTI |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | DGEISLVAGIPSECDCKNDANCDCYQSGDGYAKDAKLSAPSSLAASPDGTLYIADLGNIR |
| NOV40t | ------------------------------------------------------------ |
| NOV40u | GLLLQTAFLGTSRRVLFKYRRQTRLSEILYDSTRVSFTYDETAGVLKTVNLQSDGFICTI |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | RYRQIGPLIDRQIFRFSEDGMVNARFDYSYDNSFRVTSMQGVINETPLPIDLYQFDDISG |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | RYRQIGPLIDRQIFRFSEDGMVNARFDYSYDNSFRVTSMQGVINETPLPIDLYQFDDISG |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | IRAVSKNKPLLNSMNFYEVASPTDQELYIFDINGTHQYTVSLVTGDYLYNFSYSNDNDIT |
| NOV40t | ------------------------------------------------------------ |
| NOV40u | RYRQIGPLIDRQIFRFSEDGMVNARFDYSYDNSFRVTSMQGVINETPLPIDLYQFDDISG |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | KVEQFGKFGVIYYDINQIISTAVMTYTKHFDAHGRIKEIQYEIFRSLMYWITIQYDNMGR |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | KVEQFGKFGVIYYDINQIISTAVMTYTKHFDAHGRIKEIQYEIFRSLMYWITIQYDNMGR |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | AVTDSNGNTLRIRRDPNRMPVRVVSPDNQVIWLTIGTNGCLKGMTAQGLELVLFTYHGNS |
| NOV40t | ------------------------------------------------------------ |
| NOV40u | KVEQFGKFGVIYYDINQIISTAVMTYTKHFDAHGRIKEIQYEIFRSLMYWITIQYDNMGR |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | VTKREIKIGPFANTTKYAYEYDVDGQLQTVYLNEKIMWRYNYDLNGNLHLLNPSNSARLT |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | VTKREIKIGPFANTTKYAYEYDVDGQLQTVYLNEKIMWRYNYDLNGNLHLLNPSNSARLT |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | GLLATKSDETGWTTFFDYDSEGRLTNVTFPTGVVTNLHGDMDKAITVDIESSSREEDVSI |
| NOV40t | ------------------------------------------------------------ |
| NOV40u | VTKREIKIGPFANTTKYAYEYDVDGQLQTVYLNEKIMWRYNYDLNGNLHLLNPSNSARLT |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | PLRYDLRDRITRLGDVQYRLDEDGFLRQRGTEIFEYSSKGLLTRVYSKGSGWTVIYRYDG |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | PLRYDLRDRITRLGDVQYRLDEDGFLRQRGTEIFEYSSKGLLTRVYSKGSGWTVIYRYDG |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | TSNLSSIDSFYTMVQDQLRNSYQIGYDGSLRIIYASGLDSHYQTEPHVLAGTANPTVAKR |
| NOV40t | ------------------------------------------------------------ |
| NOV40u | PLRYDLRDRITRLGDVQYRLDEDGFLRQRGTEIFEYSSKGLLTRVYSKGSGWTVIYRYDG |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | LGRRVSSKTSLGQHLQFFYADLTYPTRITHVYNHSSSEITSLYYDLQGHLFAMEISSGDE |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | LGRRVSSKTSLGQHLQFFYADLTYPTRITHVYNHSSSEITSLYYDLQGHLFAMEISSGDE |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | NMTLPGENGQNLVEWRFRKEQAQGKVNVFGRKLRVNGRNLLSVDFDRTTKTEKIYDDHRK |
| NOV40t | ------------------------------------------------------------ |
| NOV40u | LGRRVSSKTSLGQHLQFFYADLTYPTRITHVYNHSSSEITSLYYDLQGHLFAMEISSGDE |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | FYIASDNTGTPLAVFSSNGLMLKQIQYTAYGEIYFDSNIDFQLVIGFHGGLYDPLTKLIH |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | FYIASDNTGTPLAVFSSNGLMLKQIQYTAYGEIYFDSNIDFQLVIGFHGGLYDPLTKLIH |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | FLLRIAYDTSGHPTLWLPSSKLMAVNVTYSSTGQIASIQRGTTSEKVDYDGQGRIVSRVF |
| NOV40t | ------------------------------------------------------------ |
| NOV40u | FYIASDNTGTPLAVFSSNGLMLKQIQYTAYGEIYFDSNIDFQLVIGFHGGLYDPLTKLIH |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | FGERDYDILAGRWTTPDIEIWKRIGKDPAPFNLYMFRNNNPASKIHDVKDYITDVNSWLV |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | FGERDYDILAGRWTTPDIEIWKRIGKDPAPFNLYMFRNNNPASKIHDVKDYITDVNSWLV |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | ADGKTWSYTYLEKSMVLLLHSQRQYIFEYDMWDRLSAITMPSVARHTMQTIRSIGYYRNI |
| NOV40t | ------------------------------------------------------------ |
| NOV40u | FGERDYDILAGRWTTPDIEIWKRIGKDPAPFNLYMFRNNNPASKIHDVKDYITDVNSWLV |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | TFGFHLHNAIPGFPVPKFDLTEPSYELVKSQQWDDIPPIFGVQQQVARQAKAFLSLGKMA |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | TFGFHLHNAIPGFPVPKFDLTEPSYELVKSQQWDDIPPIFGVQQQVARQAKAFLSLGKMA |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | YNPPESNASIITDYNEEGLLLQTAFLGTSRRVLFKYRRQTRLSEILYDSTRVSFTYDETA |
| NOV40t | ------------------------------------------------------------ |
| NOV40u | TFGFHLHNAIPGFPVPKFDLTEPSYELVKSQQWDDIPPIFGVQQQVARQAKAFLSLGKMA |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | EVQVSRRRAGGAQSWLWFATVKSLIGKGVMLAVSQGRVQTNVLNIANEDCIKVAAVLNNA |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | EVQVSRRRAGGAQSWLWFATVKSLIGKGVMLAVSQGRVQTNVLNIANEDCIKVAAVLNNA |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | GVLKTVNLQSDGFICTIRYRQIGPLIDRQIFRFSEDGMVNARFDYSYDNSFRVTSMQGVI |
| NOV40t | ------------------------------------------------------------ |
| NOV40u | EVQVSRRRAGGAQSWLWFATVKSLIGKGVMLAVSQGRVQTNVLNIANEDCIKVAAVLNNA |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | FYLENLHFTIEGKDTHYFIKTTTPESDLGTLRLTSGRKALENGINVTVSQSTTVVNGRTR |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | FYLENLHFTIEGKDTHYFIKTTTPESDLGTLRLTSGRKALENGINVTVSQSTTVVNGRTR |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | NETPLPIDLYQFDDISGKVEQFGKFGVIYYDINQIISTAVMTYTKHFDAHGRIKEIQYEI |
| NOV40t | ------------------------------------------------------------ |
| NOV40u | FYLENLHFTIEGKDTHYFIKTTTPESDLGTLRLTSGRKALENGINVTVSQSTTVVNGRTR |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | RFADVEMQFGALALHVRYGMTLDEEKARILEQARQRALARAWAREQQRVRDGEEGARLWT |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | RFADVEMQFGALALHVRYGMTLDEEKARILEQARQRALARAWAREQQRVRDGEEGARLWT |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | FRSLMYWITIQYDNNGRVTKREIKIGPFANTTKYAYEYDVDGQLQTVYLNEKIMWRYNYD |
| NOV40t | ------------------------------------------------------------ |
| NOV40u | RFADVEMQFGALALHVRYGMTLDEEKARILEQARQRALARAWAREQQRVRDGEEGARLWT |
| NOV40V | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | EGEKRQLLSAGKVQGYDGYYVLSVEQYPELADSANNIQFLRQSEIGRR------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | EGEKRQLLSAGKVQGYDGYYVLSVEQYPELADSANNIQFLRQSEIGRR------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | LNGNLHLLNPSNSARLTPLRYDLRDRITRLGDVQYRLDEDGFLRQRGTEIFEYSSKGLLT |
| NOV40t | ------------------------------------------------------------ |
| NOV40u | EGEKRQLLSAGKVQGYDGYYVLSVEQYPELADSANNIQFLRQSEIGRR------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | RVYSKGSGWTVIYRYDGLGRRVSSKTSLGQHLQFFYADLTYPTRITHVYNHSSSEITSLY |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | YDLQGHLFAMEISSGDEFYIASDNTGTPLAVFSSNGLMLKQIQYTAYGEIYFDSNIDFQL |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | VIGFHGGLYDPLTKLIHFGERDYDILAGRWTTPDIEIWKRIGKDPAPFNLYMFRNNNPAS |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | KIHDVKDYITDVNSWLVTFGFHLHNAIPGFPVPKFDLTEPSYELVKSQQWDDIPPIFGVQ |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | QQVARQAKAFLSLGKMAEVQVSRRRAGGAQSWLWFATVKSLIGKGVMLAVSQGRVQTNVL |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | NIANEDCIKVAAVLNNAFYLENLHFTIEGKDTHYFIKTTTPESDLGTLRLTSGRKALENG |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | INVTVSQSTTVVNGRTRRFADVEMQFGALALHVRYGMTLDEEKARILEQARQRALARAWA |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ------------------------------------------------------------ |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40g | ------------------------------------------------------------ |
| NOV40h | ------------------------------------------------------------ |
| NOV40i | ------------------------------------------------------------ |
| NOV40j | ------------------------------------------------------------ |
| NOV40k | ------------------------------------------------------------ |
| NOV40l | ------------------------------------------------------------ |
| NOV40m | ------------------------------------------------------------ |
| NOV40n | ------------------------------------------------------------ |
| NOV40o | ------------------------------------------------------------ |
| NOV40p | ------------------------------------------------------------ |
| NOV40q | ------------------------------------------------------------ |
| NOV40r | ------------------------------------------------------------ |
| NOV40s | REQQRVRDGEEGARLWTEGEKRQLLSAGKVQGYDGYYVLSVEQYPELADSANNIQFLRQS |
| NOV40t | ------------------------------------------------------------ |
| NOV40u | ------------------------------------------------------------ |
| NOV40v | ------------------------------------------------------------ |
| NOV40w | ------------------------------------------------------------ |
| NOV40x | ------------------------------------------------------------ |
| NOV40y | ------------------------------------------------------------ |
| NOV40z | ------------------------------------------------------------ |
| NOV40aa | ------------------------------------------------------------ |
| NOV40ab | ------------------------------------------------------------ |
| NOV40ac | ------------------------------------------------------------ |
| NOV40a | ----- |
| NOV40b | ----- |
| NOV40c | ----- |
| NOV40d | ----- |
| NOV40e | ----- |
| NOV40f | ----- |
| NOV40g | ----- |
| NOV40h | ----- |
| NOV40i | ----- |
| NOV40j | ----- |
| NOV40k | ----- |
| NOV40l | ----- |
| NOV40m | ----- |
| NOV40n | ----- |
| NOV40o | ----- |
| NOV40p | ----- |
| NOV40q | ----- |
| NOV40r | ----- |
| NOV40s | EIGRR |
| NOV40t | ----- |
| NOV40u | ----- |
| NOV40v | ----- |
| NOV40w | ----- |
| NOV40x | ----- |
| NOV40y | ----- |
| NOV40z | ----- |
| NOV40aa | ----- |
| NOV40ab | ----- |
| NOV40ac | ----- |
| NOV40a | (SEQ ID NO: 486) |
| NOV40b | (SEQ ID NO: 488) |
| NOV40c | (SEQ ID NO: 490) |
| NOV40d | (SEQ ID NO: 492) |
| NOV40e | (SEQ ID NO: 494) |
| NOV40f | (SEQ ID NO: 496) |
| NOV40g | (SEQ ID NO: 498) |
| NOV40h | (SEQ ID NO: 500) |
| NOV40i | (SEQ ID NO: 502) |
| NOV40j | (SEQ ID NO: 504) |
| NOV40k | (SEQ ID NO: 506) |
| NOV40l | (SEQ ID NO: 508) |
| NOV40m | (SEQ ID NO: 510) |
| NOV40n | (SEQ ID NO: 512) |
| NOV40o | (SEQ ID NO: 514) |
| NOV40p | (SEQ ID NO: 516) |
| NOV40q | (SEQ ID NO: 518) |
| NOV40r | (SEQ ID NO: 520) |
| NOV40s | (SEQ ID NO: 522) |
| NOV40t | (SEQ ID NO: 524) |
| NOV40u | (SEQ ID NO: 526) |
| NOV40v | (SEQ ID NO: 528) |
| NOV40w | (SEQ ID NO: 530) |
| NOV40x | (SEQ ID NO: 532) |
| NOV40y | (SEQ ID NO: 534) |
| NOV40z | (SEQ ID NO: 536) |
| NOV40aa | (SEQ ID NO: 538) |
| NOV40ab | (SEQ ID NO: 540) |
| NOV40ac | (SEQ ID NO: 542) |

Further analysis of the NOV40a protein yielded the following properties shown in Table 40C.

TABLE 40C
Protein Sequence Properties NOV40a

| | |
|---|---|
| SignalP analysis: | No Known Signal Sequence Indicated |

PSORT II analysis:
PSG: a new signal peptide prediction method

N-region: length 7; pos.chg 3; neg.chg 2
H-region: length 6; peak value −1.30
PSG score: −5.70

GvH: von Heijne's method for signal seq. recognition

GvH score (threshold: −2.1): −13.53
possible cleavage site: between 60 and 61

>>> Seems to have no N-terminal signal peptide
ALOM: Klein et al's method for TM region allocation

| Init position for calculation: 1 | | |
|---|---|---|
| Tentative number of TMS(s) for the threshold 0.5: 3 | | |
| Number of TMS(s) for threshold 0.5: 1 | | |
| INTEGRAL | Likelihood = −9.39 | Transmembrane 309-325 |

| | |
|---|---|
| PERIPHERAL | Likelihood = 2.54 (at 2515) |

ALOM score: −9.39 (number of TMSs: 1)

MTOP: Prediction of membrane topology (Hartmann et al.)

Center position for calculation: 316
Charge difference: −6.5 C(−1.5) − N(5.0)
N >= C: N-terminal side will be inside

>>> membrane topology: type 2 (cytoplasmic tail 1 to 309)
MITDISC: discrimination of mitochondrial targeting seq

| | | | |
|---|---|---|---|
| R content: | 0 | Hyd Moment(75): | 3.03 |
| Hyd Moment(95): | 4.83 | G content: | 0 |
| D/E content: | 2 | S/T content: | 0 |

Score: −7.62

Gavel: prediction of cleavage sites for mitochondrial preseq

cleavage site motif not found

NUCDISC: discrimination of nuclear localization signals

pat4: none
pat7: none
bipartite: RRPYCSLTKSRREKERR at 6
bipartite: RKEQAQGKVNVFGRKLR at 1778
content of basic residues: 9.9%
NLS Score: 0.51

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 57

checking 63 PROSITE DNA binding motifs:

Leucine zipper pattern (PS00029): *** found ***

LTPLRYDLRDRITRLGDVQYRL at 2196

none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 55.5

COIL: Lupas's algorithm to detect coiled-coil regions

total: 0 residues

Final Results (k = 9/23):

```
34.8%: nuclear
26.1%: mitochondrial
13.0%: Golgi
  8.7%: cytoplasmic
  4.3%: plasma membrane
  4.3%: vesicles of secretory system
  4.3%: extracellular, including cell wall
  4.3%: peroxisomal
```

>> prediction for CG55069-01 is nuc (k = 23)

A search of the NOV40a protein against the Geneseq database, a proprietary database that contains sequences published in patents and patent publication, yielded several homologous proteins shown in Table 40D.

TABLE 40D
Geneseq Results for NOV40a

| | | NOV40a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP53586 | Human NOV15a protein SEQ | 1 . . . 2725 | 2725/2725 (100%) | 0.0 |
| | ID NO: 36 - Homo sapiens, | 1 . . . 2725 | 2725/2725 (100%) | |
| | 2725 aa. [WO200262999-A2, | | | |
| | 15-AUG-2002] | | | |
| ABP53587 | Human NOV15b protein SEQ | 1 . . . 2725 | 2720/2725 (99%) | 0.0 |
| | ID NO: 38 - Homo sapiens, | 1 . . . 2721 | 2720/2725 (99%) | |
| | 2721 aa. [WO200262999-A2, | | | |
| | 15-AUG-2002] | | | |
| ABP53588 | Human NOV15c protein SEQ | 148 . . . 2725 | 2553/2579 (98%) | 0.0 |
| | ID NO: 40 - Homo sapiens, | 54 . . . 2628 | 2558/2579 (98%) | |
| | 2628 aa. [WO200262999-A2, | | | |
| | 15-AUG-2002] | | | |
| ABP53589 | Human NOV15d protein SEQ | 148 . . . 2725 | 2536/2579 (98%) | 0.0 |
| | ID NO: 42 - Homo sapiens, | 54 . . . 2613 | 2540/2579 (98%) | |
| | 2613 aa. [WO200262999-A2, | | | |
| | 15-AUG-2002] | | | |
| ABB98401 | Human NOV1, a TEN-M4 like | 1 . . . 2725 | 1917/2813 (68%) | 0.0 |
| | protein - Homo sapiens, 2794 | 1 . . . 2794 | 2260/2813 (80%) | |
| | aa. [WO200255704-A2, 18- | | | |
| | JUL-2002] | | | |

In a BLAST search of public sequence databases, the NOV40a protein was found to have homology to the proteins shown in the BLASTP data in Table 40E.

TABLE 40E
Public BLASTP Results for NOV40a

| | | NOV40a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9WTS6 | Ten-m3 - Mus musculus | 1 . . . 2725 | 2663/2725 (97%) | 0.0 |
| | (Mouse), 2715 aa. | 1 . . . 2715 | 2696/2725 (98%) | |
| Q9JLC1 | ODZ3 - Mus musculus | 354 . . . 2725 | 2308/2372 (97%) | 0.0 |
| | (Mouse), 2346 aa (fragment). | 1 . . . 2346 | 2334/2372 (98%) | |
| Q9W7R4 | Ten-m3 - Brachydanio rerio | 143 . . . 2725 | 2116/2598 (81%) | 0.0 |
| | (Zebrafish) (Danio rerio), 2590 | 27 . . . 2590 | 2352/2598 (90%) | |
| | aa. | | | |
| Q9WTS7 | Ten-m4 - Mus musculus | 1 . . . 2725 | 1867/2790 (66%) | 0.0 |
| | (Mouse), 2771 aa. | 1 . . . 2771 | 2227/2790 (78%) | |
| Q9DER5 | Teneurin-2 - Gallus gallus | 173 . . . 2725 | 1777/2561 (69%) | 0.0 |
| | (Chicken), 2802 aa (fragment). | 267 . . . 2802 | 2138/2561 (83%) | |

PFam analysis indicates that the NOV40a protein contains the domains shown in the Table 40F.

TABLE 40F
Domain Analysis of NOV40a

| Pfam | NOV40a | Identities/Similarities | Expect |
|---|---|---|---|
| Domain | Match Region | for the Matched Region | Value |
| EGF | 522 . . . 548 | 12/47 (26%) | 0.98 |
| | | 24/47 (51%) | |
| EGF | 586 . . . 613 | 10/47 (21%) | 0.43 |
| | | 19/47 (40%) | |
| EGF | 618 . . . 645 | 12/47 (26%) | 0.093 |
| | | 22/47 (47%) | |
| EGF | 652 . . . 680 | 12/47 (26%) | 0.87 |
| | | 22/47 (47%) | |
| EGF | 716 . . . 742 | 12/47 (26%) | 0.024 |
| | | 23/47 (49%) | |
| EGF | 762 . . . 792 | 11/47 (23%) | 0.66 |
| | | 21/47 (45%) | |
| NHL | 1497 . . . 1524 | 10/29 (34%) | 0.043 |
| | | 22/29 (76%) | |

### Example 41

The NOV41 clone was analyzed, and the nucleotide and encoded polypeptide sequences are shown in Table 41A.

TABLE 41A

NOV41 Sequence Analysis

| | | |
|---|---|---|
| NOV41a, CG55343-03 | SEQ ID NO: 553 | 943 bp |
| DNA Sequence | ORF Start: ATG at 2 | ORF Stop: TAA at 941 |

TATGAATTGGGTAAATGACAGCATCATACAGGAGTTTATTCTGCTGGGTTTCTCAGATCGACCTTGGC
TGGAGTTTCCACTCCTTGTGGTCTTCTTGATTTCTTACACTGTGACCATCTTTGGCAATCTGACCATT
ATTCTAGTGTCACGCCTGGACACCAAACTTCATACCCCCATGTATTTTTTTCTTACCAATCTATCACT
CCTGGATCTTTGTTACACCACATGTACAGTCCCACAAATGCTAGTAAATTTATGCAGCATCAGGAAAG
TAATCAGTTATCGTGGCTGTGTAGCCCAGCTTTTCATATTTCTGGCCTTGGGGGCTACTGAATATCTT
CTCCTGGCCGTCATGTCCTTTGATTGGTTTGTAGCTATTTGTCGGCCTCTCCATTACTCAGTTATCAT
GCACCAGAGACTCTGCCTCCAGTTGGCAGCTGCATCCTGGGTTACTGGTTTTAGTAACTCAGTGTGGT
TGTCTACCCTGACTCTCCAGCTGCCACTCTGTGACCCCTATGTGATAGATCACTTTCTCTGTGAAGTC
CCTGCACTGCTCAAGTTATCTTGTGTTGAGACAACAGCAAATGAGGCTGAACTATTCCTTGTCAGTGA
GCTCTTCCATCTAATACCCCTGACACTCATCCTTATATCATATGCTTTTATTGTCCGAGCAGTATTGA
GGATACAGTCTGCTGAAGGTCGACAAAAAGCATTTGGGACATGTGGTTCCCATCTAATTGTGGTGTCT
CTTTTTTATAGTACAGCCGTCTCTGTGTACCTGCAACCACCTTCGCCCAGCTCCAAGGACCAAGGAAA
GATGGTTTCTCTCTTCTATGGAATCATTGCACCCATGCTGAATCCCCTTATATATACACTTAGGAACA
AGGAGGTAAAGGAAGGCTTTAAAAGGTTGGTTGCAAGAGTCTTCTTAATCAAGAAATAA

| | | | |
|---|---|---|---|
| NOV41a, CG55343-03 | | | |
| Protein Sequence | SEQ ID NO: 554 | 313 aa | MW at 35443.7kD |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSAEGRQKAFGTCGSHLIVVSLFYSTAVSVYLQPPSPSSKDQGK
MVSLFYGIIAPMLNPLIYTLRNKEVKEGFKRLVARVFLIKK

| | | |
|---|---|---|
| NOV41b, 260568382 | SEQ ID NO: 555 | 946 bp |
| DNA Sequence | ORF Start: at 3 | ORF Stop: TAG at 936 |

ATTGGGTAAATGACAGCATCATACAGGAGTTTATTCTGCTGGGTTTCTCAGATCGACCTTGGCTGGAG
TTTCCACTCCTTGTGGTCTTCTTGATTTCTTACACTGTGACCATCTTTGGCAATCTGACCATTATTCT
AGTGTCACGCCTGGACACCAAACTTCATACCCCCATGTATTTTTTTCTTACCAATCTATCACTCCTGG
ATCTTTGTTACACCACATGTACAGTCCCACAAATGCTAGTAAATTTATGCAGCATCAGGAAAGTAATC
AGTTATCGTGGCTGTGTAGCCCAGCTTTTCATATTTCTGGCCTTGGGGGCTACTGAATATCTTCTCCT
GGCCGTCATGTCCTTTGATAGGTTTGTAGCTATTTGTCGGCCTCTCCATTACTCAGTTATCATGCACC
AGAGACTCTGCCTCCAGTTGGCAGCTGCATCCTGGGTTACTGGTTTTAGTAACTCAGTGTGGTTGTCT
ACCCTGACTCTCCAGCTGCCACTCTGTGACCCCTATGTGATAGATCACTTTCTCTGTGAAGTCCCTGC
ACTGCTCAAGTTATCTTGTGTTGAGACAACAGCAAATGAGGCTGAACTATTCCTTGTCAGTGAGCTCT
TCCATCTAATACCCCTGACACTCATCCTTATATCATATGCTTTTATTGTCCGAGCAGTATTGAGGATA
CAGTCTGCTGAAGGTCGACAAAAAGCATTTGGGACATGTGGTTCCCATCTAATTGTGGTGTCTCTTTT
TTATAGTACAGCCGTCTCTGTGTACCTGCAACCACCTTCGCCCAGCTCCAAGGACCAAGGAAAGATGG
TTTCTCTCTTCTATGGAATCATTGCACCCATGCTGAATCCCCTTATATATACACTTAGGAACAAGGAG
GTAAAGGAAGGCTTTAAAAGGTTGGTTGCAAGAGTCTTCTTAATCAAGAAATAGGGATTCGG

| | | | |
|---|---|---|---|
| NOV41b, 260568382 | | | |
| Protein Sequence | SEQ ID NO: 556 | 311 aa | MW at 35168.4kD |

WVNDSIIQEFILLGFSDRPVVLEFPLLVVFLISYTVTIFGNTIILVSRLDTKLHTPMYFFLTNLSLLD
LCYTTCTVPQMLVNLCSIRKVISYRGCVAQLFIFLALGATEYLLLAVMSFDRFVAICRPLHYSVIMHQ
RLCLQLAAASWVTGFSNSVWLSTLTLQLPLCDPYVIDHFLCEVPALLKLSCVETTANEAELFLVSELF
HLIPLTLILISYAFIVRAVLRIQSAEGRQKAFGTCGSHLIVVSLFYSTAVSVYLQPPSPSSKDQGKMV
SLFYGIIAPMLNPLIYTLRNKEVKEGFKRLVARVFLIKK

| | | |
|---|---|---|
| NOV41c, 314361391 | SEQ ID NO: 557 | 960 bp |
| DNA Sequence | ORF Start: at 1 | ORF Stop: end of sequence |

CGCGGATCCACCATGAATTGGGTAAATGACAGCATCATACAGGAGTTTATTTTGCTGGGTTTCTCAGA
TCGACCTTGGCTGGAGTTTCCACTCCTTGTGGTCTTCTTGATTTCTTACACTGTGACCATCTTTGGCA
ATCTGACCATTATTCTAGTGTCACGCCTGGACACCAAACTTCATACCCCCATGTATTTTTTTCTTACC
AATCTATCACTCCTGGATCTTTGTTACACCACATGTACAGTCCCACAAATGCTAGTAAATTTATGCAG
CATCAGGAAAGTAATCAGTTATCGTGGCTGTGTAGCCCAGCTTTTCATATTTCTGGCCAAGGGGGCTA
CTGAATATCTTCTCCTGGCCGTCATGTCCTTTGATAGGTTTGTAGCTATTTGTCGGCCTCTCCATTAC
TCAGTTATCATGCACCAGAGACTCTGCCTCCAGTTGGCAGCTGCATCCTGGGTTACTGGTTTTAGTAA
CTCAGTGTGGTTGTCTACCCTGACTCTCCAGCTGCCACTCTGTGACCCCTATGTGATAGATCACTTTC
TCTGTGAAGTCCCTGCACTGCTCAAGTTATCTTGTGTTGAGACAACAGCAAATGAGGCTGAACTATTC
CTTGTCAGTGAGCTCTTCCATCTAATACCCCTGACACTCATCCTTATATCATATGCTTTTATTGTCCG
AGCAGTATTGAGGATACAGTCTGCTGAAGGTCGACAAAAAGCATTTGGGACATGTGGTTCCCATCTAA
TTGTGGTGTCTCTTTTTTATAGTACAGCCGTCTCTGTGTACCTGCAACCACCTTCGCCCAGCTCCAAG
GACCAAGGAAAGATGGTTTCTCTCTTCTATGGAATCATTGCACCCATGCTGAATCCCCTTATATATAC
ACTTAGGAACAAGGAGGTAAGGAAGGCTTTAAAAGGTTGGTTGCAAGAGTCTTCTTAATCAAGAAAC
TCGAGGCG

| | | | |
|---|---|---|---|
| NOV41c, 314361391 | | | |
| Protein Sequence | SEQ ID NO: 558 | 320 aa | MW at 36128.5kD |

RGSTMNWVNDSIIQEFILLGFSDRPWLEFPLLVVFLISYTVTIFGNLTIILVSRLDTKLHTPMYFFLT
NLSLLDLCYTTCTVPQMLVNLCSIRKVISYRGCVAQLFIFLALGATEYLLLAVMSFDRFVAICRPLHY
SVIMHQRLCLQLAAASWVTGFSNSVWLSTLTLQLPLCDPYVIDHFLCEVPALLKLSCVETTANEAELF
LVSELFHLIPLTLILISYAFIVRAVLRIQSAEGRQKAFGTCGSHLIVVSLFYSTAVSVYLQPPSPSSK
DQGKMVSLFYGIIAPMLNPLIYTLRNKEVKEGFKRLVARVFLIKKLEA

| | | |
|---|---|---|
| NOV41d, 317286137 | SEQ ID NO: 559 | 321 bp |
| DNA Sequence | ORF Start: at 1 | ORF Stop: end of sequence |

CGCGGATCCACCATGAATTGGGTAAATGACAGCATCATACAGGAGTTTATTCTGCTGGGTTTCTCAGA
TCGACCTTGGCTGGAGTTTCCACTCCTTGTGGTCTTCTTGATTTCTTACACTGTGACCATCTTTGGCA
ATCTGACCATTATTCTAGTGTCACGCCTGGACACCAAACTTCATACCCCCATGTATTTTTTTCTTACC
AATCTATCACTCCTGGATCTTTGTTACACCACATGTACAGTCCCACAAATGCTAGTAAATTTATGCAG
CATCAGGAAAGTAATCAGTTATCGTGGCTGTGTAGCCCAGCTCGAGGCG

| | | | |
|---|---|---|---|
| NOV41d, 317286137 | | | |
| Protein Sequence | SEQ ID NO: 560 | 107 aa | MW at 12270.4kD |

RGSTMNWVNDSIIQEFILLGFSDRPWLEFPLLVVFLISYTVTIFGNLTIILVSRLDTKLHTPMYFFLT
NLSLLDLCYTTCTVPQMLVNLCSIRKVISYRGCVAQLEA

| | | |
|---|---|---|
| NOV41e, CG55343-01 | SEQ ID NO: 561 | 943 bp |
| DNA Sequence | ORF Start: ATG at 2 | ORF Stop: TAA at 941 |

TATGAATTGGGTAAATGACAGCATCATACAGGAGTTTATTCTGCTGGGTTTCTCAGATCGACCTTGGC
TGGAGTTTCCACTCCTTGTGGTCTTCTTGATTTCTTACACTGTGACCATCTTTGGCAATCTGACCATT
ATTCTAGTGTCACGCCTGGACACCAAACTTCATACCCCCATGTATTTTTTTCTTACCAATCTATCACT
CCTGGATCTTTGTTACACCACATGTACAGTCCCACAAATGCTAGTAAATTTATGCAGCATCAGGAAAG
TAATCAGTTATCGTGGCTGTGTAGCCCAGCTTTTCATATTTCTGGCCTTGGGGGCTACTGAATATCTT
CTCCTGGCCGTCATGTCCTTTGATTGGTTTGTAGCTATTTGTCGGCCTCTCCATTACTCAGTTATCAT
GCACCAGAGACTCTGCCTCCAGTTGGCAGCTGCATCCTGGGTTACTGGTTTTAGTAACTCAGTGTGGT
TGTCTACCCTGACTCTCCAGCTGCCACTCTGTGACCCCTATGTGATAGATCACTTTCTCTGTGAAGTC
CCTGCACTGCTCAAGTTATCTTGTGTTGAGACAACAGCAAATGAGGCTGAACTATTCCTTGTCAGTGA
GCTCTTCCATCTAATACCCCTGACACTCATCCTTATATCATATGCTTTTATTGTCCGAGCAGTATTGA
GGATACAGTCTGCTGAAGGTCGACAAAAAGCATTTGGGACATGTGGTTCCCATCTAATTGTGGTGTCT
CTTTTTTATAGTACAGCCGTCTCTGTGTACCTGCAACCACCTTCGCCCAGCTCCAAGGACCCGGAAAA
GATGGTTTCTCTCTTCTATGGAATCATTGCACCCATGCTGAATCCCCTTATATATACACTTAGGAACA
AGGAGGTAAAGGAAGGCTTTAAAAGGTTGGTTGCAAGAGTCTTCTTAATCAAGAAATAA

| | | | |
|---|---|---|---|
| NOV41e, CG55343-01 | | | |
| Protein Sequence | SEQ ID NO: 562 | 313 aa | MW at 35443.7kD |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWSTLTLQLPLCDPYVIADHFLCEVPALLKLSCVETTANEAELFLVSE
LFHLIPLTLILISYAFIVRAVLRIQSABGRQKAFGTCGSHLIVVSLFYSTAVSVYLQPPSPSSKDQGK
MVSLFYGIIAPMLNPLIYTLRNKEVKBGFKRLVARVFLIKK

| | | |
|---|---|---|
| NOV41f, CG55343-02 | SEQ ID NO: 563 | 933 bp |
| DNA Sequence | ORF Start: ATG at 15 | ORF Stop: TAA at 954 |

TTGAAACATTAATCATGAATTGGGTAAATGACAGCATCATACAGGAGTTTATTCTGCTGGGTTTCTCA
GATCGACCTTGGCTGGAGTTTCCACTCCTTGTGGTCTTCTTGATTTCTTACACTGTGACCATCTTTGG
CAATCTGACCATTATTCTAGTGTCACGCCTGGACACCAACTTCATACCCCCAATGTATTTTTTTCTTA
CCAATCTATCACTCCTGGATCTTTGTTACACCACATGTACAGTCCCACAAATGCTAGTAAATTTATGC
AGCATCAGGAAAGTAATCAGTTATCGTGGCTGTGTAGCCCAGCTTTTCATATTTCTGGCCTTGGGGGC
TACTGAATATCTTCTCCTGGCCGTCATGTCCTTTGATAGGTTTGTAGCTATTTGTCGGCCTCTCCATT
ACTCAGTTATCATGCACCAGAGACTCTGCCTCCAGTTGGCAGCTGCATCCTGGGTTACTGGTTTTAGT
AACTCAGTGTGGTTGTCTACCCTGACTCTCCAGCTGCCACTCTGTGACCCCTATGTGATAGATCACTT
TCTCTGTGAAGTCCCTGCACTGCTCAAGTTATCTTGTGTTGAGACAACAGCAAATGAGGCTGAACTAT
TCCTTGATCAGTGAGCTCTTCCATCTAATACCCCTGACACTCATCCTTATATGTATGCTTTTATTGTC
CGAGCAGTATTGAGGATACAGTCTGCTGAAGGTCGACAAAAAGCATTTGGGACATGTGGTTCCCATCT
AATTGTGGTGTCTCTTTTTTATAGTACAGCCGTCTCTGTGTACCTGCAACCACCTTCGCCCAGCTCCA
AGGACCAAGGAAAGATGGTTTCTCTCTTCTATGGAATCATTGCACCCATGCTGAATCCCCTTATATAT
ACACTTAGGAACAAGGAGGTAAAGGAAGGCTTTAAAAGGTTGGTTGCAAGAGTCTTCTTAATCAAGAA
ATAAGAAATATGCAAATGATAAGCTTTGCTAAAGACAAAAT

| | | | |
|---|---|---|---|
| NOV41f, CG55343-02 | | | |
| Protein Sequence | SEQ ID NO: 564 | 313 aa | MW at 35413.7kD |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDRFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSAEGRQKAFGTCGSHLIVVSLFYSTAVSVYLQPPSPSSKDQGK
MVSLFYGIIAPMLNPLIYTLRNKEVKEGFKRLVARVFLIKK

| | | |
|---|---|---|
| NOV41g, CG55343-04 | SEQ ID NO: 565 | 60 bp |
| DNA Sequence | ORF Start: at 1 | ORF Stop: end of sequence |

GACAGCATCATACAGGAGTTTATTCTGCTGGGTTTCTCAGATCGACCTTGGCTGGAGTTT

| | | | |
|---|---|---|---|
| NOV41g, CG55343-04 | | | |
| Protein Sequence | SEQ ID NO: 566 | 20 aa | MW at 2425.7kD |

DSIIQEFILLGFSDRPWLEF

| | | |
|---|---|---|
| NOV41h, CG55343-05 | SEQ ID NO: 567 | 60 bp |
| DNA Sequence | ORF Start: at 1 | ORF Stop: end of sequence |

GAAGTCCCTGCACTGCTCAAGTTATCTTGTGTTGAGACAACAGCAAATGAGGCTGAACTA

| | | | |
|---|---|---|---|
| NOV41h, GG55343-05 | | | |
| Protein Sequence | SEQ ID NO: 567 | 20 aa | MW at 2130.4kD |

EVPALLKLSCVETTANEAEL

| | | |
|---|---|---|
| NOV41i, CG55343-06 | SEQ ID NO: 569 | 946 bp |
| DNA Sequence | ORF Start: at 3 | ORF Stop: TAG at 936 |

ATTGGGTAAATGACAGCATCATACAGGAGTTTATTCTGCTGGGTTTCTCAGATCGACCTTGGCTGGAG
TTTCCACTCCTTGTGGTCTTCTTGATTTCTTACACTGTGACCATCTTTGGCAATCTGACCATTATTCT
AGTGTCACGCCTGGACACCAAACTTCATACCCCCATGTATTTTTTTCTTACCAATCTATCACTCCTGG
ATCTTTGTTACACCACATGTACAGTCCCACAAATGCTAGTAAATTTATGCAGCATCAGGAAAGTAATC
AGTTATCGTGGCTGTGTAGCCCAGCTTTTCATATTTCTGCCTTGGGGGCTACTGAATATACTTCTCCT
GGCCGTCATGTCCTTTGATAGGTTTGTAGCTATTTGTCGGCCTCTCCATTACTCAGTTATCATGCACC
AGAGACTCTGCCTCCAGTTGGCAGCTGCATCCTGGGTTACTGGTTTTAGTAACTCAGTGTGGTTGTCT
ACCCTGACTCTCCAGCTGCCACTCTGTGACCCCTATGTGATAGATCACTTTCTCTGTGAAGTCCCTGC
ACTGCTCAAGTTATCTTGTGTTGAGACAACAGCAAATGAGGCTGAACTATTCCTTGTCAGTGAGCTCT
TCCATCTAATACCCCTGACACTCATCCTTATATCATATGCTTTTATTGTCCGAGCAGTATTGAGGATA
CAGTCTGCTGAAGGTCGACAAAAAGCATTTGGGACATGTGGTTCCCATCTAATTGTGGTGTCTCTTTT
TTATAGTACAGCCGTCTCTGTGTACCTGCAACCACCTTCGCCCAGCTCCAAGGACCAAGGAAAGATGG
TTTCTCTCTTCTATGGAATCATTGCACCCATGCTGAATCCCCTTATATATACACTTAGGAACAAGGAG
GTAAAGGAAGGCTTTAAAAGGTTGGTTGCAAGAGTCTTCTTAATCAAGAAATAGGGATTCGG

| | | | |
|---|---|---|---|
| NOV41i, CG55343-06 | | | |
| Protein Sequence | SEQ ID NO: 570 | 311 aa | MW at 35168.4kD |

WVNDSIIQEFILLGFSDRPWLEFPLLVVFLISYTVTIFGNLTIILVSRLDTKLHTPMYFFLTNLSLLD
LCYTTCTVPQMLVNLCSIRKVISYRGCVAQLFIFLALGATEYLLLAVMSFDRFVAICRPLHYSVIMHQ
RLCLQLAAASWVTGFSNSVWLSTLTLQLPLCDPYVIDHFLCEVPALLKLSCVETTANEAELFLVSELF
HLIPLTLILISYAFIVRAVLRIQSABGRQKAFGTCGSHLIVVSLFYSTAVSVYLQPPSPSSKDQGKMV
SLFYGIIAPMLNPLIYTLRNKEVKEGFKRLVARVFLIKK

| | | |
|---|---|---|
| NOV41j, | | |
| SNP13373740 of | SEQ ID NO: 571 | 943 bp |
| CG55343-03, | ORF Start: ATG at 2 | ORF Stop: TAA at 941 |
| DNA Sequence | SNP Pos: 568 | SNP Change: T to C |

TATGAATTGGGTAAATGACAGCATCATACAGGAGTTTATTCTGCTGGGTTTCTCAGATCGACCTTGGC
TGGAGTTTCCACTCCTTGTGGTCTTCTTGATTTCTTACACTGTGACCATCTTTGGCAATCTGACCATT
ATTCTAGTGTCACGCCTGGACACCAAACTTCATACCCCCATGTATTTTTTTCTTACCAATCTATCACT
CCTGGATCTTTGTTACACCACATGTACAGTCCCACAAATGCTAGTAAATTTATGCAGCATCAGGAAAG
TAATCAGTTATCGTGGCTGTGTAGCCCAGCTTTTCATATTTCTGGCCTTGGGGGCTACTGAATATCTT
CTCCTGGCCGTCATGTCCTTTGATTGGTTTGTAGCTATTTGTCGGCCTCTCCATTACTCAGTTATCAT
GCACCAGAGACTCTGCCTCCAGTTGGCAGCTGCATCCTGGGTTACTGGTTTTAGTAACTCAGTGTGGT
TGTCTACCCTGACTCTCCAGCTGCCACTCTGTGACCCCTATGTGATAGATCACTTTCTCTGTGAAGTC
CCTGCACTGCTCAAGTTATCTTGCGTTGAGACAACAGCAAATGAGGCTGAACTATTCCTTGTCAGTGA
GCTCTTCCATCTAATACCCCTGACACTCATCCTTATATCATATGCTTTTATTGTCCGAGCAGTATTGA
GGATACAGTCTGCTGAAGGTCGACAAAAAGCATTTGGGACATGTGGTTCCCATCTAATTGTGGTGTCT
CTTTTTTATAGTACAGCCGTCTCTGTGTACCTGCAACCACCTTCGCCCAGCTCCAAGGACCAAGGAAA
GATGGTTTCTCTCTTCTATGGAATCATTGCACCCATGCTGAATCCCCTTATATATACACTTAGGAACA
AGGAGGTAAAGGAAGGCTTTAAAAGGTTGGTTGCAAGAGTCTTCTTAATCAAGAAATAA

| | | | |
|---|---|---|---|
| NOV41j, | | | |
| SNP13373740 of | | | |
| CG55343-03, | SEQ ID NO: 572 | | MW at 35443.7kD |
| Protein Sequence | SNP Pos: 189 | 313 aa | SNP Change: Cys to Cys |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSAEGRQKAFGTCGSHLIVVSLFYSTAVSVYLQPPSPSSKDQGK
MVSLFYGIIAPMLNPLIYTLRNKEVKEGFKRLVARVFLIKK

| | | |
|---|---|---|
| NOV41k, | | |
| SNP13376425 of | SEQ ID NO: 573 | 943 bp |
| CG55343-03, | ORF Start: ATG at 2 | ORF Stop: TAA at 941 |
| DNA Sequence | SNP Pos: 758 | SNP Change: A to G |

TATGAATTGGGTAAATGACAGCATCATACAGGAGTTTATTCTGCTGGGTTTCTCAGATCGACCTTGGC
TGGAGTTTCCACTCCTTGTGGTCTTCTTGATTTCTTACACTGTGACCATCTTTGGCAATCTGACCATT
ATTCTAGTGTCACGCCTGGACACCAAACTTCATACCCCCATGTATTTTTTTCTTACCAATCTATCACT
CCTGGATCTTTGTTACACCACATGTACAGTCCCACAAATGCTAGTAAATTTATGCAGCATCAGGAAAG
TAATCAGTTATCGTGGCTGTGTAGCCCAGCTTTTCATATTTCTGGCCTTGGGGGCTACTGAATATCTT
CTCCTGGCCGTCATGTCCTTTGATTGGTTTGTAGCTATTTGTCGGCCTCTCCATTACTCAGTTATCAT
GCACCAGAGACTCTGCCTCCAGTTGGCAGCTGCATCCTGGGTTACTGGTTTTAGTAACTCAGTGTGGT
TGTCTACCCTGACTCTCCAGCTGCCACTCTGTGACCCCTATGTGATAGATCACTTTCTCTGTGAAGTC
CCTGCACTGCTCAAGTTATCTTGTGTTGAGACAACAGCAAATGAGGCTGAACTATTCCTTGTCAGTGA
GCTCTTCCATCTAATACCCCTGACACTCATCCTTATATCATATGCTTTTATTGTCCGAGCAGTATTGA
GGATACAGTCTGCTGAAGGTCGACAAAAAGCATTTGGGACATGTGGTTCCCATCTAATTGTGGTGTCT
CTTTTTTATGGTACAGCCGTCTCTGTGTACCTGCAACCACCTTCGCCCAGCTCCAAGGACCAAGGAAA
GATGGTTTCTCTCTTCTATGGAATCATTGCACCCATGCTGAATCCCCTTATATATACACTTAGGAACA
AGGAGGTAAAGGAAGGCTTTAAAAGGTTGGTTGCAAGAGTCTTCTTAATCAAGAAATAA

| | | | |
|---|---|---|---|
| NOV41k, | | | |
| SNP13376425 of | | | |
| CG55343-03, | SEQ ID NO: 574 | | MW at 35413.7kD |
| Protein Sequence | SNP Pos: 253 | 313 aa | SNP Change: Set to Gly |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSAEGRQKAFGTCGSHLIVVSLFYGTAVSVYLQPPSPSSKDQGK
MVSLFYGIIAPMLNPLIYTLRNKEVKEGFKRLVARVFLIKK

| | | |
|---|---|---|
| NOV41l, | | |
| SNP13376424 of | SEQ ID NO: 575 | 943 bp |
| CG55343-03, | ORF Start: ATG at 2 | ORF Stop: TAA at 941 |
| DNA Sequence | SNP Pos: 810 | SNP Change: A to G |

TATGAATTGGGTAAATGACAGCATCATACAGGAGTTTATTCTGCTGGGTTTCTCAGATCGACCTTGGC
TGGAGTTTCCACTCCTTGTGGTCTTCTTGATTTCTTACACTGTGACCATCTTTGGCAATCTGACCATT
ATTCTAGTGTCACGCCTGGACACCAAACTTCATACCCCCATGTATTTTTTTCTTACCAATCTATCACT
CCTGGATCTTTGTTACACCACATGTACAGTCCCACAAATGCTAGTAAATTTATGCAGCATCAGGAAAG
TAATCAGTTATCGTGGCTGTGTAGCCCAGCTTTTCATATTTCTGGCCTTGGGGGCTACTGAATATCTT
CTCCTGGCCGTCATGTCCTTTGATTGGTTTGTAGCTATTTGTCGGCCTCTCCATTACTCAGTTATCAT
GCACCAGAGACTCTGCCTCCAGTTGGCAGCTGCATCCTGGGTTACTGGTTTTAGTAACTCAGTGTGGT
TGTCTACCCTGACTCTCCAGCTGCCACTCTGTGACCCCTATGTGATAGATCACTTTCTCTGTGAAGTC
CCTGCACTGCTCAAGTTATCTTGTGTTGAGACAACAGCAAATGAGGCTGAACTATTCCTTGTCAGTGA
GCTCTTCCATCTAATACCCCTGACACTCATCCTTATATCATATGCTTTTATTGTCCGAGCAGTATTGA
GGATACAGTCTGCTGAAGGTCGACAAAAAGCATTTGGGACATGTGGTTCCCATCTAATTGTGGTGTCT
CTTTTTTATAGTACAGCCGTCTCTGTGTACCTGCAACCACCTTCGCCCAGCTCCAAGGACCGAGGAAA
GATGGTTTCTCTCTTCTATGGAATCATTGCACCCATGCTGAATCCCCTTATATATACACTTAGGAACA
AGGAGGTAAAGGAAGGCTTTAAAAGGTTGGTTGCAAGAGTCTTCTTAATCAAGAAATAA

| | | | |
|---|---|---|---|
| NOV41l, | | | |
| SNP13376424 of | | | |
| CG55343-03, | SEQ ID NO: 576 | | MW at 35471.8kD |
| Protein Sequence | SNP Pos: 270 | 313 aa | SNP Change: Gln to Arg |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSABGRQKAFGTCGSHLIVVSLFYSTAVSVYLQPPSPSSKDRGK
MVSLFYGIIAPMLNPLIYTLRNKEVKEGFKRLVARVFLIKK

| | | |
|---|---|---|
| NOV41m, | | |
| SNP13376423 of | SEQ ID NO: 577 | 943 bp |
| CG55343-03, | ORF Start: ATG at 2 | ORF Stop: TAA at 941 |
| DNA Sequence | SNP Pos: 908 | SNP Change: A to G |
| TATGAATTGGGTAAATGACAGCATCATACAGGAGTTTATTCTGCTGGGTTTCTCAGATCGACCTTGGC | | |
| TGGAGTTTCCACTCCTTGTGGTCTTCTTGATTTCTTACACTGTGACCATCTTTGGCAATCTGACCATT | | |
| ATTCTAGTGTCACGCCTGGACACCAAACTTCATACCCCCATGTATTTTTTTCTTACCAATCTATCACT | | |
| CCTGGATCTTTGTTACACCACATGTACAGTCCCACAAATGCTAGTAAATTTATGCAGCATCAGGAAAG | | |
| TAATCAGTTATCGTGGCTGTGTAGCCCAGCTTTTCATATTTCTGGCCTTGGGGGCTACTGAATATCTT | | |
| CTCCTGGCCGTCATGTCCTTTGATTGGTTTGTAGCTATTTGTCGGCCTCTCCATTACTCAGTTATCAT | | |
| GCACCAGAGACTCTGCCTCCAGTTGGCAGCTGCATCCTGGGTTACTGGTTTTAGTAACTCAGTGTGGT | | |
| TGTCTACCCTGACTCTCCAGCTGCCACTCTGTGACCCCTATGTGATAGATCACTTTCTCTGTGAAGTC | | |
| CCTGCACTGCTCAAGTTATCTTGTGTTGAGACAACAGCAAATGAGGCTGAACTATTCCTTGTCAGTGA | | |
| GCTCTTCCATCTAATACCCCTGACACTCATCCTTATATCATATGCTTTTATTGTCCGAGCAGTATTGA | | |
| GGATACAGTCTGCTGAAGGTCGACAAAAAGCATTTGGGACATGTGGTTCCCATCTAATTGTGGTGTCT | | |
| CTTTTTTATAGTACAGCCGTCTCTGTGTACCTGCAACCACCTTCGCCCAGCTCCAAGGACCAAGGAAA | | |
| GATGGTTTCTCTCTTCTATGGAATCATTGCACCCATGCTGAATCCCCTTATATATACACTTAGGAACA | | |
| AGGAGGTAAAGGAAGGCTTTAAAGGGTTGGTTGCAAGAGTCTTCTTAATCAAGAAATAA | | |

| | | | |
|---|---|---|---|
| NOV41m, | | | |
| SNP13376423 of | | | |
| CG55343-03, | SEQ ID NO: 578 | | MW at 35344.6kD |
| Protein Sequence | SNP Pos: 303 | 313 aa | SNP Change: Arg to Gly |

MNWVNDSIIQEFILLGFSDRPWLEFPLLVVFLISYTVTIFGNLTIILVSRLDTKLHTPMYFFLTNLSL
LDLCYTTCTVPQMLVNLCSIRKVISYRGCVAQLFIFLALGATEYLLLAVMSFDWFVAICRPLHYSVIM
HQRLCLQLAAASWVTGFSNSVWLSTLTLQLPLCDPYVIDHFLCEVPALLKLSCVETTANEAELFLVSE
LFHLIPLTLILISYAFIVRAVLRIQSAEGRQKAFGTCGSHLIVVSLFYSTAVSVYLQPPSPSSKDQGK
MVSLFYGIIAPMLNPLIYTLRNKEVKEGFKGLVARVFLIKK

A ClustalW comparison of the above protein sequences yields the following sequence alignment shown in Table 41B.

TABLE 41B

Comparison of the NOV41 protein sequences.

| | |
|---|---|
| NOV41a | ----MNWVNDSIIQEFILLGFSDRPWLEFPLLVVFLISYTVTIFGNLTIILVSRLDTKLH |
| NOV41b | ------WVNDSIIQEFILLGFSDRPWLEFPLLVVFLISYTVTIFGNLTIILVSRLDTKLH |
| NOV41c | RGSTMNWVNDSIIQEFILLGFSDRPWLEFPLLVVFLISYTVTIFGNLTIILVSRLDTKLH |
| NOV41d | RGSTMNWVNDSIIQEFILLGFSDRPWLEFPLLVVFLISYTVTIFGNLTIILVSRLDTKLH |
| NOV41e | ----MNWVNDSIIQEFILLGFSDRPWLEFPLLVVFLISYTVTIFGNLTIILVSRLDTKLH |
| NOV41f | ----MNWVNDSIIQEFILLGFSDRPWLEFPLLVVFLISYTVTIFGNLTIILVSRLDTKLH |
| NOV41g | ---------DSIIQEFILLGFSDRPWLEF------------------------------- |
| NOV41h | ------------------------------------------------------------ |
| NOV41i | ------WVNDSIIQEFILLGFSDRPWLEFPLLVVFLISYTVTIFGNLTIILVSRLDTKLH |
| NOV41a | TPMYFFLTNLSLLDLCYTTCTVPQMLVNLCSIRKVISYRGCVAQLFIFLALGATEYLLLA |
| NOV41b | TPMYFFLTNLSLLDLCYTTCTVPQMLVNLCSIRKVISYRGCVAQLFIFLALGATEYLLLA |
| NOV41c | TPMYFFLTNLSLLDLCYTTCTVPQMLVNLCSIRKVISYRGCVAQLFIFLALGATEYLLLA |
| NOV41d | TPMYFFLTNLSLLDLCYTTCTVPQMLVNLCSIRKVISYRGCVAQLEA------------- |
| NOV41e | TPMYFFLTNLSLLDLCYTTCTVPQMLVNLCSIRKVISYRGCVAQLFIFLALGATEYLLLA |
| NOV41f | TPMYFFLTNLSLLDLCYTTCTVPQMLVNLCSIRKVISYRGCVAQLFIFLALGATEYLLLA |
| NOV41g | ------------------------------------------------------------ |
| NOV41h | ------------------------------------------------------------ |
| NOV41i | TPMYFFLTNLSLLDLCYTTCTVPQMLVNLCSIRKVISYRGCVAQLFIFLALGATEYLLLA |
| NOV41a | VMSFDWFVAICRPLHYSVIMHQRLCLQLAAASWVTGFSNSVWLSTLTLQLPLCDPYVIDH |
| NOV41b | VMSFDRFVAICRPLHYSVIMHQRLCLQLAAASWVTGFSNSVWLSTLTLQLPLCDPYVIDH |
| NOV41c | VMSFDRFVAICRPLHYSVIMHQRLCLQLAAASWVTGFSNSVWLSTLTLQLPLCDPYVIDH |
| NOV41d | ------------------------------------------------------------ |
| NOV41e | VMSFDWFVAICRPLHYSVIMHQRLCLQLAAASWVTGFSNSVWLSTLTLQLPLCDPYVIDH |
| NOV41f | VMSFDRFVAICRPLHYSVIMHQRLCLQLAAASWVTGFSNSVWLSTLTLQLPLCDPYVIDH |
| NOV41g | ------------------------------------------------------------ |
| NOV41h | ------------------------------------------------------------ |
| NOV41i | VMSFDRFVAICRPLHYSVIMHQRLCLQLAAASWVTGFSNSVWLSTLTLQLPLCDPYVIDH |
| NOV41a | FLCEVPALLKLSCVETTANEAELFLVSELFHLIPLTLILISYAFIVRAVLRIQSAEGRQK |
| NOV41b | FLCEVPALLKLSCVETTANEAELFLVSELFHLIPLTLILISYAFIVRAVLRIQSAEGRQK |
| NOV41c | FLCEVPALLKLSCVETTANEAELFLVSELFHLIPLTLILISYAFIVRAVLRIQSAEGRQK |
| NOV41d | ------------------------------------------------------------ |
| NOV41e | FLCEVPALLKLSCVETTANEAELFLVSELFHLIPLTLILISYAFIVRAVLRIQSAEGRQK |
| NOV41f | FLCEVPALLKLSCVETTANEAELFLVSELFHLIPLTLILISYAFIVRAVLRIQSAEGRQK |
| NOV41g | ------------------------------------------------------------ |
| NOV41h | ---EVPALLKLSCVETTANEAEL------------------------------------- |
| NOV41i | FLCEVPALLKLSCVETTANEAELFLVSELFHLIPLTLILISYAFIVRAVLRIQSAEGRQK |
| NOV41a | AFGTCGSHLIVVSLFYSTAVSVYLQPPSPSSKDQGKMVSLFYGIIAPMLNPLIYTLRNKE |
| NOV41b | AFGTCGSHLIVVSLFYSTAVSVYLQPPSPSSKDQGKMVSLFYGIIAPMLNPLIYTLRNKE |
| NOV41c | AFGTCGSHLIVVSLFYSTAVSVYLQPPSPSSKDQGKMVSLFYGIIAPMLNPLIYTLRNKE |
| NOV41d | ------------------------------------------------------------ |
| NOV41e | AFGTCGSHLIVVSLFYSTAVSVYLQPPSPSSKDQGKMVSLFYGIIAPMLNPLIYTLRNKE |
| NOV41f | AFGTCGSHLIVVSLFYSTAVSVYLQPPSPSSKDQGKMVSLFYGIIAPMLNPLIYTLRNKE |
| NOV41g | ------------------------------------------------------------ |
| NOV41h | ------------------------------------------------------------ |
| NOV41i | AFGTCGSHLIVVSLFYSTAVSVYLQPPSPSSKDQGKMVSLFYGIIAPMLNPLIYTLRNKE |
| NOV41a | VKEGFKRLVARVFLIKK--- |
| NOV41b | VKEGFKRLVARVFLIKK--- |
| NOV41c | VKEGFKRLVARVFLIKKLEA |
| NOV41d | -------------------- |
| NOV41e | VKEGFKRLVARVFLIKK--- |
| NOV41f | VKEGFKRLVARVFLIKK--- |
| NOV41g | -------------------- |
| NOV41h | -------------------- |
| NOV41i | VKEGFKRLVARVFLIKK--- |
| NOV41a | (SEQ ID NO: 554) |
| NOV41b | (SEQ ID NO: 556) |
| NOV41c | (SEQ ID NO: 558) |
| NOV41d | (SEQ ID NO: 560) |
| NOV41e | (SEQ ID NO: 562) |
| NOV41f | (SEQ ID NO: 564) |
| NOV41g | (SEQ ID NO: 566) |
| NOV41h | (SEQ ID NO: 568) |
| NOV41i | (SEQ ID NO: 570) |

Further analysis of the NOV41a protein yielded the following properties shown in Table 41C.

TABLE 41C
Protein Sequence Properties NOV41a

| | |
|---|---|
| SignalP analysis: | Cleavage site between residues 39 and 40 |
| PSORT II analysis: | |

| PSG: | a new signal peptide prediction method | | | |
|---|---|---|---|---|
| | N-region: | length 11; | pos.chg 0; | neg.chg 2 |
| | H-region: | length 7; | peak value | 0.00 |
| | PSG score: | −4.40 | | |

| | |
|---|---|
| GvH: | von Heijne's method for signal seq. recognition |
| | GvH score (threshold: −2.1): −4.39 |
| | possible cleavage site: between 38 and 39 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood =−4.78 | Transmembrane 25-41 |
| | INTEGRAL | Likelihood =−5.15 | Transmembrane 101-117 |
| | INTEGRAL | Likelihood =−5.47 | Transmembrane 206-222 |
| | INTEGRAL | Likelihood =−0.80 | Transmembrane 244-260 |
| | INTEGRAL | Likelihood =−0.75 | Transmembrane 273-289 |

| | |
|---|---|
| PERIPHERAL | Likelihood = 3.02 (at 173) |

| | ALOM score: −5.47 (number of TMSs: 5) |
|---|---|
| MTOP: | Prediction of membrane topology (Hartmann et al.) |
| | Center position for calculation: 32 |
| | Charge difference: 3.5 C(1.5) − N(−2.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 8.26 |
| Hyd Moment(95): | 10.83 | G content: | 0 |
| D/E content: | 2 | S/T content: | 1 |
| Score: −5.89 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 7.3%
NLS Score: −0.47

KDEL: ER retention motif in the C-terminus: none
ER Membrane Retention Signals:

KKXX-like motif in the C-terminus: FLIK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
22.2%: vacuolar
11.1%: Golgi
11.1%: mitochondrial

>> prediction for CG55343-03 is end (k = 9)

A search of the NOV41a protein against the Geneseq database, a proprietary database that contains sequences published in patents and patent publication, yielded several homologous proteins shown in Table 41D.

TABLE 41D
Geneseq Results for NOV41a

| | | NOV41a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE09961 | G-protein coupled-receptor | 1 . . . 313 | 313/313 (100%) | e−180 |
| | (GPCR)NOV1b protein - | 1 . . . 313 | 313/313 (100%) | |
| | Unidentified, 313 aa. | | | |
| | [WO200166746-A2, 13-SEP- | | | |
| | 2001] | | | |
| AAU95518 | Human olfactory and pheromone | 1 . . . 313 | 312/313 (99%) | e−178 |
| | G protein-coupled receptor #5 - | 1 . . . 313 | 312/313 (99%) | |
| | Homo sapiens, 313 aa. | | | |
| | [WO200224726-A2, 28-MAR- | | | |
| | 2002] | | | |
| ABP95746 | Human GPCR polypeptide SEQ | 1 . . . 313 | 312/313 (99%) | e−178 |
| | ID NO 302 - Homo sapiens, 313 | 1 . . . 313 | 312/313 (99%) | |
| | aa. [WO200216548-A2, 28-FEB- | | | |
| | 2002] | | | |
| AAE09960 | G-protein coupled-receptor | 1 . . . 313 | 312/313 (99%) | e−178 |
| | (GPCR) NOV1a protein - | 1 . . . 313 | 312/313 (99%) | |
| | Unidentified, 313 aa. | | | |
| | [WO200166746-A2, 13-SEP- | | | |
| | 2001] | | | |
| AAG71842 | Human olfactory receptor | 1 . . . 313 | 312/313 (99%) | e−178 |
| | polypeptide, SEQ ID NO: 1523 - | 1 . . . 313 | 312/313 (99%) | |
| | Homo sapiens, 313 aa. | | | |
| | [WO200127158-A2, 19-APR- | | | |
| | 2001] | | | |

In a BLAST search of public sequence databases, the NOV41a protein was found to have homology to the proteins shown in the BLASTP data in Table 41E.

TABLE 41E
Public BLASTP Results for NOV41a

| | | NOV41a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P58173 | Olfactory receptor 2B6 (Hs6M1- | 1 . . . 313 | 312/313 (99%) | e−178 |
| | 32) (Olfactory receptor 6-31) | 1 . . . 313 | 312/313 (99%) | |
| | (OR6-31) - Homo sapiens | | | |
| | (Human), 313 aa. | | | |
| Q8VFH1 | Olfactory receptor MOR256-11 - | 1 . . . 312 | 254/312 (81%) | e−145 |
| | Mus musculus (Mouse), 313 aa. | 1 . . . 312 | 273/312 (87%) | |
| Q9GZK3 | Olfactory receptor 2B2 (Olfactory | 1 . . . 310 | 252/310 (81%) | e−143 |
| | receptor 6-1) (OR6-1) (Hs6M1- | 1 . . . 310 | 277/310 (89%) | |
| | 10) - Homo sapiens (Human), 357 aa. | | | |
| Q8VFH2 | Olfactory receptor MOR256-10 - | 1 . . . 313 | 247/313 (78%) | e−140 |
| | Mus musculus (Mouse), 313 aa. | 1 . . . 313 | 275/313 (86%) | |
| Q63394 | OL1 receptor - Rattus norvegicus | 1 . . . 313 | 244/313 (77%) | e−139 |
| | (Rat), 313 aa. | 1 . . . 313 | 273/313 (86%) | |

PFam analysis indicates that the NOV41a protein contains the domains shown in the Table 41F.

TABLE 41F
Domain Analysis of NOV41a

| Pfam | NOV41a | Identities/Similarities | Expect |
|---|---|---|---|
| Domain | Match Region | for the Matched Region | Value |
| 7tm_1 | 41 . . . 290 | 56/276 (20%) | 1.3e−33 |
| | | 176/276 (64%) | |
| TAS2R | 99 . . . 308 | 51/309 (17%) | 0.27 |
| | | 126/309 (41%) | |

### Example 42

The NOV42 clone was analyzed, and the nucleotide and encoded polypeptide sequences are shown in Table 42A.

TABLE 42A

NOV42 Sequence Analysis

| | | |
|---|---|---|
| NOV42a, CG55358-04 | SEQ ID NO: 579 | 947 bp |
| DNA Sequence | ORF Start: ATG at 2 | ORF Stop: TAG at 938 |

TATGGAAAACGATAATACAAGTTCTTTCGAAGGCTTCATCCTGGTGGGCTTCTCTGATCGTCCCCACC
TAGAGCTGATCGTCTTTGTGGTTGTCCTCATCTTTTATCTGCTGACTCTTCTTGGCAACATGACCATT
GTCTTGCTTTCAGCTCTGGATTCCCGGCTGCACACACCAATGTATTTCTTTTTGGCAAACCTCTCATT
CCTGGACATGTGTTTCACCACAGGTTCCATCCCTCAGATGCTCTACAACCTTTGGGGTCCAGATAAGA
CCATCAGCTATGTGGGTTGTGCCATCCAGCTGTACTTTGTCCTGGCCCTGGGAGGGGTGGAGTGTGTC
CTCCTGGCTGTCATGGCATATGACCGCTATGCTGCAGTCTGCAAACCCCTGCACTACACCATCATCAT
GCACCCACGTCTCTGTGGACAGCTGGCTTCAGTGGCATGGCTGAGTGGCTTTGGCAATTCTCTCATAA
TGGCACCCCAGACATTGATGCTACCCCGCTGTGGGCACAGACGAGTTGACCACTTTCTCTGTGAGATG
CCAGCACTAATTGGTATGGCCTGTGTAGACACCATGATGCTTGAGGCACTGGCTTTTGCCCTGGCAAT
CTTTATCATCCTGGCACCACTCATCCTCATTCTCATTTCTTATGGTTACGTTGGAGGAACAGTGCTTA
GGATCAAGTCAGCTGCTGGGCGAAAGAAAGCCTTCAACACTTGCAGCTCGCATCTAATTGTTGTCTCT
CTCTTCTATGGTACAATCATATACATGTACCTCCAGCCAGCAAATACTTATTCCCAGGACCAGGGCAA
GTTTCTTACCCTTTTCTACACAATTGTCACTCCCAGTGTTAACCCCTTGATCTATACACTAAGAAACA
AAGATGTTAAAGAGGCCATGAAGAAGGTGCTAGGGAAGGGGAGTGCAGAAATATAGTAAGGGA

| | | | |
|---|---|---|---|
| NOV42a, | | | |
| CG55358-04 | | | |
| Protein Sequence | SEQ ID NO: 580 | 312 aa | MW at 34640.9kD |

MENDNTSSFEGFILVGFSDRPHLELIVFVVVLIFYLLTLLGNMTIVLLSALDSRLHTPMYFFLANLSF
LDMCFTTGSIPQMLYNLWGPDKTISYVGCAIQLYFVLALGGVECVLLAVMAYDRYAAVCKPLHYTIIM
HPRLCGQLASVAWLSGFGNSLIMAPQTLMLPRCGHRRVDHFLCEMPALIGMACVDTMMLEALAFALAI
FIVLAPLILILISYGYVGGTVLRIKSAAGRKKAFNTCSSHLIVVSLFYGTIIYMYLQPANTYSODQGK
FLTLFYTIVTPSVNPLIYTLRNKDVKEAMKKVLGKGSAEI

| | | |
|---|---|---|
| NOV42b, CG55358-03 | SEQ ID NO: 581 | 932 bp |
| DNA Sequence | ORF Start: at 3 | ORF Stop: TAG at 924 |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGCTATGTG
GGTTGTGCCATCCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42b, CG55358-03 | | | |
| Protein Sequence | SEQ ID NO: 582 | 307 aa | MW at 34051.3kD |
| TSSFEGFILVGFSDRPHLELIVFVVVLIFYLLTLLGNNTIVLLSALDSRLHTPMYFFLANLSFLDMCF | | | |
| TTGSIPQMLYNLWGPDKTISYVGCAIQLYFVLALGGVECVLLAVMAYDRYAAVCKPLHYTIIMHPRLC | | | |
| GQLASVAWLSGFGNSLIMAPQTLMLPRCGHRRVDHFLCEMPALIGMACVDTMMLEALAFALAIFIILA | | | |
| PLILILISYGYVGGTVLRIKSAAGRKKAFNTCSSHLIVVSLFYGTIIYMYLQPANTYSQDQGKFLTLF | | | |
| YTIVTPSVNPLIYTLRNKDVKEANKKVLGKGSAEI | | | |

| | | |
|---|---|---|
| NOV42c, 317863291 | SEQ ID NO: 583 | 693 bp |
| DNA Sequence | ORF Start: at 1 | ORF Stop: end of sequence |

CGCGGATCCACCATGTACAAGTTCTTTCGAGGCTTCATCCTGGTGGGCTTCTCTGATCGTCCCCACCT
AGAGCTGATCGTCTTTGTGGTTGTCCTCATCTTTTATCTGCTGACTCTTCTTGGCAACATGACCATTG
TCTTGCTTTCAGCTCTGGATTCCCGGCTGCACACACCAATGTATTTCTTTTTGGCAAACCTCTCATTC
CTGGACATGTGTTTCACCACAGGTTCCATCCCTCAGATGCTCTACAACCTTTGGGGTCCAGATAAGAC
CATCAGCTATGTGGGTTGTGCCATCCAGCTGTACTTTGTCCTGCCCTGGGAGGGGTGGAGTGTAGTCC
TCCTGGCTGTCATGGCATATGACCGCTATGCTGCAGTCTGCAAACCCCTGCACTACACCATCATCATG
CACCCACGTCTCTGTGGACAGCTGGTTTCAGTGGCATGGCTGAGTGGCTTTGGCAATTCTCTCATAAT
GGCACCCCAGACATTGATGCTACCCCGCTGTGGGCACAGACGAGTTGACCACTTTCTCTGTGAGATGC
CAGCACTAATTGGTATGGCCTGTGTAGACACCATGATGCTTGAGGCACTGGCTTTTGCCCTGGCAATC
TTTATCATCCTGGCACCACTCATCCTCATTCTCATTTCTTATGGTTACGTTGGAGGAACAGTGCTTAG
GATCCTCGAGGCG

| | | | |
|---|---|---|---|
| NOV42c, 317863291 | | | |
| Protein Sequence | SEQ ID NO: 584 | 231 aa | MW at 25750.8kD |

RGSTMYKFFRGFILVGFSDRPHLELIVFVVVLIFYLLTLLGNMTIVLLSALDSRLHTPMYFFLANSLF
LDMCFTTGSIPQMLYNLWGPDKTISYVGCAIQLYFVALGGVECVLLAVMAYDRYAAVCKPLHYTAIIM
HPRLCGQLVSVAWLSGFGNSLIMAPQTLMLPRCGHRRVDHFLCEMPALIGMACVDTMMLEALAFALAI
FlILAPLILILISYGYVGGTVLRILEA

| | | |
|---|---|---|
| NOV42d, 317863328 | SEQ ID NO: 585 | 609 bp |
| DNA Sequence | ORF Start: at 1 | ORF Stop: end of sequence |

CGCGGATCCCTCATCTTTTATCTGCTGACTCTTCTTGGCAACATGACCATTGTCTTGCTTTCAGCTCT
GGATTCCCGGCTGCACACACCAATGTATTTCTTTTTGGCAAACCTCTCATTCCTGGACATGTGTTTCA
CCACAGGTTCCATCCCTCAGATGCTCTACAACCTTTGGGGTCCAGATAAGACCATCAGCTATGTGGGT
TGTGCCATCCAGCTGTACTTTGTCCTGGCCCTGGGAGGGGTGGAGTGTGTCCTCCTGGCTGTCATGGC
ATATGACCGCTATGCTGCAGTCTGCAAACCCCTGCACTACACCATCATCATGCACCCACGTCTCTGTG
GACAGCTGGTTTCAGTGGCATGGCTGAGTGGCTTTGGCAATTCTCTCATAATGGCACCCCAGACATTG
ATGCTACCCCGCTGTGGGGCACAGACGAGTTGACCACTTTCTCTGTGAGATGCAGCACTAATTGGTAT
GGCCTGTGTAGACACCATGATGCTTGAGGCACTGGCTTTTGCCCTGGCAATCTTTATCATCCTGGCAC
CACTCATCCTCATTCTCATTTCTTATGGTTACGTTGGAGGAACAGTGCTTAGGATCCTCGAGGCG

| | | | |
|---|---|---|---|
| NOV42d, 317863328 | | | |
| Protein Sequence | SEQ ID NO: 586 | 203 aa | MW at 22437.8kD |

RGSLIFYLLTLLGNMTIVLLSALDSRLHTPMYFFLANLSFLDMCFTTGSIPQMLYNLWGPDKTISYVG
CAIQLYFVLALGGVECVLLAVMAYDRYAAVCKPLHYTIIMHPRLCGQLVSVAWLSGFGNSLIMAPQTL
MLPRCGHRRVDHFLCEMPALIGMACVDTMMLEALAFALAIFIILAPLILILISYGYVGGTVLRILEA

| | | |
|---|---|---|
| NOV42e, 317863350 | SEQ ID NO: 587 | 433 bp |
| DNA Sequence | ORF Start: at 1 | ORF Stop: at 433 |

CGCGGATCCGCCATCCAGCTGTACTTTGTCCTGGCCCTGGGAGGGGTGGAGTGTGTCCTCCTGGCTGT
CATGGCATATGACCGCTATGCTGCAGTCTGCAAACCCCTGCACTACACCATCATCATGCACCCACGTC
TCTGTGGACAGCTGGTTTCATGGCATGGCTGAGTGGCTTTGGCAATTCTCTCATAATGGCACCCCAAG
ACATTGATGCTACCCCGCTGTGGGCACAGACGAGTTGACCACTTTCTCTGTGAGATGCCAGCACTAAT
TGGTATGGCCTGTGTAGACACCATGATGCTTGAGGCACTGGCTTTTGCCCTGGCAATCTTTATCATCC
TGGCACCACTCATCCTCATTCTCATTTCTTATGGTTACGTTGGAGGAACAGTGCTTAGGATCCTCGAG
GCGAAGGGCGAATTCCAGCACACTG

| | | | |
|---|---|---|---|
| NOV42e, 317863350 | | | |
| Protein Sequence | SEQ ID NO: 588 | 144 aa | MW at 15783.9kD |

FGSAIQLYFVLALGGVECVLLAVMAYDRYAAVCKPLHYTIIMHPRLCGQLVSVAWLSGFGNSLIMAPQ
TLMLPRCGHRRVDHFLCEMPALIGMACVDTMMLEALAFALAIFIILAPLILILISYGYVGGTVARILE
AKGEFQHT

| | | |
|---|---|---|
| NOV42f, 271624076 | SEQ ID NO: 589 | 953 bp |
| DNA Sequence | ORF Start: at 1 | ORF Stop: TAG at 934 |

GAAAACGATAATACAAGTTCTTTCGAAGGCTTCATCCTGGTGGGCTTCTCTGATCGTCCCCACCTAGA
GCTGATCGTCTTTGTGGTTGTCCTCATCTTTTATCTGCTGACTCTTCTTGGCAACATGACCATTGTCT
TGCTTTCAGCTCTGGATTCCCGGCTGCACACACCAATGTATTTCTTTTTGGCAAACCTCTCATTCCTG
GACTGTGTTTCACCACAGGTTCCATCCCTCAGATGCTCTACAACCTTTGGGGTCCAGATAAGGACCAT
CAGCTATGTGGGTTGTGCCATCCAGCTGTACTTTGTCCTGGCCCTGGGAGGGGTGGAGTGTGTCCTCC
TGGCTGTCATGGCATATGACCGCTATGCTGCAGTCTGCAAACCCCTGCACTACACCATCATCATGCAC
CCACGTCTCTGTGGACAGCTGGCTTCAGTGGCATGGCTGAGTGGCTTTGGCAATTCTCTCATAATGGC
ACCCCAGACATTGATGCTACCCCGCTGTGGGCACAGACGAGTTGACCACTTTCTCTGTGAGATGCAAG
CACTAATTGGTATGGCCTGTGTAGACACCATGATGCTTGAGGCACTGGCTTTTGCCCTGGCAATCTTT
ATCATCCTGGCACCACTCATCCTCATTCTCATTTCTTATGGTTACGTTGGAGGAACAGTGCTTAGGAT
CAAGTCAGCTGCTGGGCGAAAGAAAGCCTTCAACACTTGCAGCTCGCATCTTTTTGTTGTCTCTCTCT
TCTATGGTACAATCATATACATGTACCTCCAGCCAGCAAATACTTATTCCCAGGACCAGGGCAAGTTT
CTTACCCTTTTCTACACAATCGTCACTCCCAGTGTTAACCCCCTGATCTATACACTAAGAAACAAAGA
TGTTAAAGAGGCCATGAAGAAGGTGCTAGGGAAGGGGAGTGCAGAAATATAGGAATTCGGAAGGGCGA
A

| | | | |
|---|---|---|---|
| NOV42f, 271624076 | | | |
| Protein Sequence | SEQ ID NO: 590 | 311 aa | MW at 34523.7kD |

ENDNTSSFEGFILVGFSDRPHLELIVFVVVLIFYLLTLLGNMTIVLLSALDSRLHTPMYFFLANLSFL
DMCFTTGSIPQMLYNLWGPDKTISYVGCAIQLYFVLALGGVECVLLAVMAYDRYAAVCKPLHYTIIMH
PRLCGQLASVAWLSFGNSLIMAPQTLMLPRCGHRRVDHFLCEMPALIGMACVDTMMLEALAFAALAIF
IILAPLILILISYGYVGGTVLRIKSAAGRKKAFNTCSSHLIVVSLFYGTIIYMYLQPANTYSQDQGKF
LTLFYTIVTPSVNPLIYTLRNKDVKEAMKKVLGKGSAEI

| | | |
|---|---|---|
| NOV42g, CG55358-01 | SEQ ID NO: 591 | 945 bp |
| DNA Sequence | ORF Start: ATG at 1 | ORF Stop: TAG at 937 |

ATGGAAAACGATAATACAAGTTCTTTCGAAGGCTTCATCCTGGTGGGCTTCTCTGATCGTCCCCACCT
AGAGCTGATCGTCTTTGTGGTTGTCCTCATCTTTTATCTGCTGACTCTTCTTGGCAACATGACCATTG
TCTTGCTTTCAGCTCTGGATTCCCGGCTGCACACACCAATGTATTTCTTTTTGGCAAACCTCTCATTC
CTGGACATGTGTTTCACCACAGGTTCCATCCCTCAGATGCTCTACAACCTTTGGGGTCCAGATAAGAC
CATCAGCTATGTGGGTTGTGCCATCCAGCTGTACTTTGTCCTGGCCCTGGGAGGGGTGGAGTGTGTCC
TCCTGGCTGTCATGGCATATGACCGCTATGCTGCAGTCTGCAAACCCCTGCACTACACCATCATCATG
CACCCACGTCTCTGTGGACAGCTGGCTTCAGTGGCATGGCTGAGTGGCTTTGGCAATTCTCTCATAAT
GGCACCCCAGACATTGATGCTACCCCGCTGTGGGCACAGACGAGTTGACCACTTTCTCTGTGAGATGC
CAGCACTAATTGGTATGGCCTGTGTAGACACCATGATGCTTGAGGCACTGGCTTTTGCCCTGGCAATC
TTTATCATCCTGGCACCACTCATCCTCATTCTCATTTCTTATGGTTACGTTGGAGGAACAGTGCTTAG
GATCAAGTCAGCTGCTGGGCGAAAGAAAGCCTTCAACACTTGCAGCTCGCATCTAATTGTTGTCTCTC
TCTTCTATGGTACAATCATATACATGTACCTCCAGCCAGCAAATACTTATTCCCAGGACCAGGGCAAG
TTTCTTACCCTTTTCTACACAATTGTCACTCCCAGTGTTAACCCCCTGATCTATACACTAAGAAACAA
AGATGTTAAAGAGGCCATGAAGAAGGTGCTAGGGAAGGGGAGTGCAGAAATATAGTAAGGG

| | | | |
|---|---|---|---|
| NOV42g, CG55358-01 | | | |
| Protein Sequence | SEQ ID NO: 592 | 312 aa | MW at 34654.9kD |

MENDNTSSFEGFILVGFSDRPHLELIVFVVVLIFYLLTLLGNMTIVLLSALDSRLHTPMYFFLANLSF
LDMCFTTGSIPQMLYNLWGPDKTISYVGCAIQLYFVLALGGVECVLLAVMAYDRYAAVCKPLHYTIIM
HPRLCGQLASVAWLSGFGNSLIMAPQTLMLPRCGHRRVDHFLCEMPALIGMACVDTMMLEALAFALAI
FIILAPLILILISYGYVGGTVLRIKSAAGRKKAFNTCSSHLIVVSLFYGTIIYNYLQPANTYSQDQGK
FLTLFYTIVTPSVNPLIYTLRNKDVKEAMKKVLGKGSAEI

| | | |
|---|---|---|
| NOV42h, CG55358-02 | SEQ ID NO: 593 | 947 bp |
| DNA Sequence | ORF Start: ATG at 3 | ORF Stop: TAG at 939 |

GTATGGAAAACGATAATACAAGTTCTTTCGAAGGCTTCATCCTGGTGGGCTTCTCTGATCGTCCCCAC
CTAGAGCTGATCGTCTTTGTGGTTGTCCTCATCTTTTATCTGCTGACTCTTCTTGGCAACATGACCAT
TGTCTTGCTTTCAGCTCTGGATTCCCGGCTGCACACACCAATGTATTTCTTTTTGGCAAACCTCTCAT
TCCTGGACATGTGTTTCACCACAGGTTCCATCCCTCAGATGCTCTACAACCTTTGGGGTCCAGATAAG
ACCATCAGCTATGTGGGTTGTGCCATCCAGCTGTACTTTGTCCTGGCCCTGGGAGGGGTGGAGTGTGT
CCTCCTGGCTGTCATGGCATATGACCGCTATGCTGCAGTCTGCAAACCCCTGCACTACACCATCATCA
TGCACCCACGTCTCTGTGGACAGCTGGCTTCAGTGGCATGGCTGAGTGGCTTTGGCAATTCTCTCATA
ATGGCACCCCAGACATTGATGCTACCCCGCTGTGGGCACAGACGAGTTGACCACTTTCTCTGTGAGAT
GCCAGCACTAATTGGTATGGCCTGTGTAGACACCATGATGCTTGAGGCACTGGCTTTTGCCCTGGCAA
TCTTTATCATCCTGGCACCACTCATCCTCATTCTCATTTCTTATGGTTACGTTGGAGGAACAGTGCTT
AGGATCAAGTCAGCTGCTGGGCGAAAGAAAGCCTTCAACACTTGCAGCTCGCATCTAATTGTTGTCTC
TCTCTTCTATGGTACAATCATATACATGTACCTCCAGCCAGCAAATACTTATTCCCAGGACCAGGGCA
AGTTTCTTACCCTTTTCTACACAATTGTCACTCCCAGTGTTAACCCCCTGATCTATACACTAAGAAAC
AAAGATGTTAAAGAGGCCATGAAGAAGGTGCTAGGGAAGGGGAGTGCAGAAATATAGTAAGGG

| | | | |
|---|---|---|---|
| NOV42h, CG55358-02 | | | |
| Protein Sequence | SEQ ID NO: 594 | 312 aa | MW at 34654.9kD |

MENDNTSSFEGFILVGFSDRPHLELIVFVVVLIFYLLTLLGNMTIVLLSALDSRLHTPMYFFLANLSF
LDMCFTTGSIPQMLYNLWGPDKTISYVGCAIQLYFVLALGGVECVLLAVMAYDRYAAVCKPLHYTIIM
HPRLCGQLASVAWLSGFGNSLIMAPQTLMLPRCGHRRVDHFLCEMPALIGMACVDTMMLEALAFALAI
FlILAPLILILISYGYVGGTVLRIKSAAGRKKAFNTCSSHLIVVSLFYGTIIYMYLQPANTYSQDQGK
FLTLFYTIVTPSVNPLIYTLRNKDVKEANKKVLGKGSAEI

| | | |
|---|---|---|
| NOV42i, CG55358-05 | SEQ ID NO: 595 | 63 bp |
| DNA Sequence | ORF Start: ATG at 1 | ORF Stop: end of sequence |

ATGGAAAACGATAATACAAGTTCTTTCGAAGGCTTCATCCTGGTGGGCTTCTCTGATCGTCCC

| | | | |
|---|---|---|---|
| NOV42i, CG55358-05 | | | |
| Protein Sequence | SEQ ID NO: 596 | 21 aa | MW at 2362.5kD |

MENDNTSSFEGFILVGFSDRP

| | | |
|---|---|---|
| NOV42j, GG55358-06 | SEQ ID NO: 597 | 60 bp |
| DNA Sequence | ORF Start: at 1 | ORF Stop: end of sequence |

ACATTGATGCTACCCCGCTGTGGGCACAGACGAGTTGACCACTTTCTCTGTGAGATGCCA

| | | | |
|---|---|---|---|
| NOV42j, CG55358-06 | | | |
| Protein Sequence | SEQ ID NO: 598 | 20 aa | MW at 2411.9kD |

TLMLPRCGHRRVDHFLCEMP

| | | |
|---|---|---|
| NOV42k, GG55358-07 | SEQ ID NO: 599 | 953 bp |
| DNA Sequence | ORF Start: at 1 | ORF Stop: TAG at 934 |

GAAAACGATAATACAAGTTCTTTCGAAGGCTTCATCCTGGTGGGCTTCTCTGATCGTCCCCACCTAGA
GCTGATCGTCTTTGTGGTTGTCCTCATCTTTTATCTGCTGACTCTTCTTGGCAACATGACCATTGTCT
TGCTTTCAGCTCTGGATTCCCGGCTGCACACACCAATGTATTTCTTTTTGGCAAACCTCTCATTCCTG
GACATGTGTTTCACCACAGGTTCCATCCCTCAGATGCTCTACAACCTTTGGGGTCCAGATAAGACCAT
CAGCTATGTGGGTTGTGCCATCCAGCTGTACTTTGTCCTGGCCCTGGGGAGGGTGGAGTGTGTCCTCC
TGGCTGTCATGGCATATGACCGCTATGCTGCAGTCTGCCAAACCCCTGCACTACACCATCATCATGGC
CCACGTCTCTGTGGACAGCTGGCTTCAGTGGCATGGCTGAGTGGCTTTGGCAATTCTCTCATAATGGC
ACCCCAGACATTGATGCTACCCCGCTGTGGGCACAGACGAGTTGACCACTTTCTCTGTGAGATGCCAG
CACTAATTGGTATGGCCTGTGTAGACACCATGATGCTTGAGGCACTGGCTTTTGCCCTGGCAATCTTT
ATCATCCTGGCACCACTCATCCTCATTCTCATTTCTTATGGTTACGTTGGAGGAACAGTGCTTAGGAT
CAAGTCAGCTGTGGGCGAAAGAAAGCCTTCAACACTTGCAGTCTCGCATCTAATTGTTGTCTCTCTCT
TCTATGGTACAATCATATACATGTACCTCCAGCCAGCAAATACTTATTCCCAGGACCAGGGCAAGTTT
CTTACCCTTTTCTACACAATCGTCACTCCCAGTGTTAACCCCCTGATCTATACACTAAGAAACAAAGA
TGTTAAAGAGGCCATGAAGAAGGTGCTAGGGAAGGGGAGTGCAGAAATATAGGAATTCGGAAGGGCGA
A

| | | | |
|---|---|---|---|
| NOV42k, CG55358-07 | | | |
| Protein Sequence | SEQ ID NO: 600 | 311 aa | MW at 34523.7kD |

ENDNTSSFEGFILVGFSDRPHLELIVFVVVLIFYLLTLLGNMTIVLLSALDSRLHTPMYFFLANLSFL
DMCFTTGSIPQMLYNLWGPDKTISYVGCAIQLYFVLALGGVECVLLAVMAYDRYAAVCKPLHYTIIMH
PRLCGQLASVAWLSGFGNSLIMAPQTLMLPRCGHRRVDHFLCEMPALIGMACVDTMMLEALAFALAIF
IILAPLILILISYGYVGGTVLRIKSAAGRKKAFNTCSSHLIVVSLFYGTIIYMYLQPANTYSQDQGKF
LTLFYTIVTPSVNPLIYTLRNKDVKEAMKKVLGKGSAEI

| | | |
|---|---|---|
| NOV42l, | | +TL,26 |
| SNP13375122 of | SEQ ID NO: 601 | 932 bp |
| CG55358-03, | ORF Start: at 3 | ORF Stop: TAG at 924 |
| DNA Sequence | SNP Pos: 64 | SNP Change: T to C |

ATACAAGTTCTTTCGAAGGCTTCATCCTGGTGGGCTTCTCTGATCGTCCCCACCTAGAGCTGACCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
GGTTGTGCCATCCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42L, | | | |
| SNP13375122 of | | | |
| CG55358-03, | SEQ ID NO: 602 | | MW at 34039.2kD |
| Protein Sequence | SNP Pos: 21 | 307 aa | SNP Change: Ile to Thr |

TSSFEGFILVGFSDRPHLELTee VFVVVLIFYLLTLLGNMTIVLLSALDSRLHTPMYFFLANLSFLDMCF
TTGSIPQMLYNLWGPDKTISYVGCAIQLYFVLALGGVECVLLAVMAYDRYAAVCKPLHYTIIMHPRLC
GQLASVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTIIYMYLQPANTYSQDQGKFLTLF

| | | |
|---|---|---|
| NOV42m | | |
| CG55358-03, | ORF Start: at 3 | ORF Stop: TAG at 924 |
| DNA Sequence | SNP Pos: 124 | SNP Change: T to C |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGCCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGAAATGTG
GGTTGTGCCATCCAGCTGTACTTTGTCCTGGCCCTGGGAGGGGTGGAGTGTGTCCTCCTAACTGTCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTAA
TATGGCCTGTGTAGACACCATGATGCTTGAGGCACTGGCTTTTGCCCTGGGGTCTTTATCATCCTTAA
CACCACTCATCCTCATTCTCATTTCTTATGGTTACTTGGAGGAACAGTGCTTAGGATCAAAGTCAGCT
GCTGGGCGAAAGAAAGCCTTCAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42m, | | | |
| SNP13375 123 of | | | |
| CG55358-03, | SEQ ID NO: 604 | | MW at 34023.2kD |
| Protein Sequence | SNP Pos: 41 | 307 aa | SNP Change: Val to Ala |

TSSFEGFILVGFSDRPHLELIVFVVVLIFYLLTLLGNMTIAee LLSALDSRLHTPMYFFLANLSFLDMCF
TTGSIPQMLYNLWGPDKTISYVGCAIQLYFVLALGGVECVLLAVMAYDRYAAVCKPLHYTIINHPRLC
GQLASVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTIIYMYLQPANTYSQDQGKFLTLF
YTIVTPSVNPLIYTLRNKDVKEANKKVLGKGSAEI

| | | |
|---|---|---|
| NOV42n, | | |
| SNP13382494 of | SEQ ID NO: 605 | 932 bp |
| CG55358-03, | ORF Start: at 3 | ORF Stop: TAG at 924 |
| DNA Sequence | SNP Pos: 254 | SNP Change: T to C |

ATACAAGTTCTTTCGAAGGCTTCATCCTGGTGGGCTTCTCTGATCGTCCCCACCTAAGGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGACee AAGACCATCAGCTATGTG
GGTTGTGCCATCCAGCTGTACTTTGTCCTGGCCCTGGGAGGGGTAAAGTGTGTCCTCCTAACTGTCAT
GGCATATGACCGCTATGCTGCAGTCTGCAAACCCCTGCACTACACAATCATCATGCACCAACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTACCCCCTGATCTATACACTAAGAAAACAAAGATGTTAAACAG
GCCATGAAGAAGGTGCTAGGGAAGGGGAGTGCAGAAATATAGTAAGGG

| | | | |
|---|---|---|---|
| NOV42n, | | | |
| SNP13382494 of | | | |
| CG55358-03, | SEQ ID NO: 606 | | MW at 34051.3kD |
| Protein Sequence | SNP Pos: 84 | 307 aa | SNP Change: Asp to Asp |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCAIQLYFVLALGGVECVLLAVMAYDRYAAVCKPLHYTIINHPRLC
GQLASVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTIIYMYLQPANTYSQDQGKFLTLF
YTIVTPSVNPLIYTLRNKDVKEAMKKVLGKGSAEI

| | | |
|---|---|---|
| NOV42o, | | |
| SN1P13375124 of | SEQ ID NO: 607 | 932 bp |
| GG55358-03, | ORF Start: at 3 | ORF Stop: TAG at 924 |
| DNA Sequence | SNP Pos: 283 | SNP Change: T to C |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCAAAGTCTTGCTTTCAGC
TCTGGATTCCCGGCTGCACACACCAATGTATTTCTTTTTGGCAAACCTCTCATTCCTGGAAATGTGTT
TCACCACAGGTTCCATCCCTCAGATGCTCTACAACCTTTGGGGTCCAGATAAGACCATAAGCTATGTG
GGTTGTGCCACee CCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCAAATGGCAACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAAGAAAGCCTTCAACACTTGCAGTCTCGCATCTAATTGTTGTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42o, | | | |
| SNP13375124 of | | | |
| CG55358-03, | SEQ ID NO: 608 | | MW at 34039.2kD |
| Protein Sequence | SNP Pos: 94 | 307 aa | SNP Change: Ile to Thr |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCATQLYFVLALGGVECVLLMTMAYDRYAAVCKPLHYTIIMHPRLC
GQLASVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTIIYMYLQPANTYSQDQGKFLTLF
YTIVTPSVNPLIYTLRNKDVKEANKKVLGKGSAEI

| | | |
|---|---|---|
| NOV42p, | | |
| SNP13375125 of | SEQ ID NO: 609 | 932 bp |
| CG55358-03, | ORF Start: at 3 | ORF Stop: TAG at 924 |
| DNA Sequence | SNP Pos: 336 | SNP Change: G to A |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGCTATGTG
GGTTGTGCCATCCAGCTGTACTTTGTCCTGGCCCTGGGAGGGGTGGAGTGTGTCCTCCTGGCTATCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCCTGGCTTTTGCCCTGGCAATCTTTATCATCCTTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42p, | | | |
| SNP13375125 of | | | |
| CG55358-03, | SEQ ID NO: 610 | | MW at 34065.3kD |
| Protein Sequence | SNP Pos: 112 | 307 aa | SNP Change: Val to Ile |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCAIQLYFVLALGGVECVLLAIMAYDRYAAVCKPLHYTIIMHPRLC
GQLASVAWLSGFGNSLIMAPQTLMLPRCGHRVDHFLCEMPALIGMACVDTMMLEALAAFALAIFIILA
PLILILISYGYVGGTVLRIKSAAGRKKAFNTCSSHLIVVSLFYGTIIYMYLQPANTYSQDQGKFLTLF
YTIVTPSVNPLIYTLRNKDVKEANKKVLGKGSAEI

| | | |
|---|---|---|
| NOV42q, | | |
| SNP13376426 of | SEQ ID NO: 611 | 932 bp |
| CG55358-03, | ORF Start: at 3 | ORF Stop: TAG at 924 |
| DNA Sequence | SNP Pos: 415 | SNP Change: A to G |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGCTATGTG
GGTTGTGCCATCCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACGGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTATTGTTGTCTCTCTCTTCTAATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCTGATCTATACACTAAGAAACAAAGATGTTAAAAGAG
GCCATGAAGAAGGTGCTAGGGAAGGGGAGTGCAGAAATATAGTAAGGG

| | | | |
|---|---|---|---|
| NOV42q, | | | |
| SNP13376426 of | | | |
| CG55358-03, | SEQ ID NO: 612 | | MW at 34079.3kD |
| Protein Sequence | SNP Pos: 138 | 307 aa | SNP Change: Gln to Arg |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCAIQLYFVLALGGVECVLLAVMAYDRYAAVCKPLHYTIIMHPRLC
GRLASVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTIIYMYLQPANTYSQDQGKFLTLF
YTIVTPSVNPLIYTLRNKDVKEAMKKVLGKGSAEI

| | | |
|---|---|---|
| NOV42r, | | |
| SNP13376427 of | SEQ ID NO: 613 | 932 bp |
| CG55358-03, | ORF Start: at 3 | ORF Stop: TAG at 924 |
| DNA Sequence | SNP Pos: 421 | SNP Change: C to T |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGCTATGTG
GGTTGTGCCATCCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACGGCTGGTTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTTCAACACTTGCAGCTCGCATCTATTGTTGTCTCTCTCTTCTAATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCTGATCTATACACTAAGAAACAAAGATGTTAAAAGAG
GCCATGAAGAAGGTGCTAGGGAAGGGGAGTGCAGAAATATAGTAAGGG

| | | | |
|---|---|---|---|
| NOV42r, | | | |
| SNP13376427 of | | | |
| CG55358-03, | SEQ ID NO: 614 | | MW at 34079.3kD |
| Protein Sequence | SNP Pos: 140 | 307 aa | SNP Change: Ala to Val |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCAIQLYFVLALGGVBCVLIAVMAYDRYAAVCKPLHYTIIMHPRLC
GQLVSVAWLSGFGNSLIMAPQTLMLPRCGHRRVDHFLCEMPALIGMACVDTMMLEALAFALAIFIILA
PLILILISYGYVGGTVLRIKSAAGRKKAFNTCSSHLIVVSLFYGTITYMYLQPANTYSQDQGKFLTLF
YTIVTPSVNPLIYTLRNKDVKEANKKVLGKGSAEI

| | | |
|---|---|---|
| NOV42s, | | |
| SNP13375126 of | SEQ ID NO: 615 | 932 bp |
| CG55358-03, | ORF Start: at 3 | ORF Stop: TAG at 924 |
| DNA Sequence | SNP Pos: 700 | SNP Change: T to C |

ATACAAGTTCTTTCGAAGGCTTCATCCTGGTGGGCTTCTCTGATCGTCCCCACCTAGAGCTGATCGTC
TTTGTGGTTGTCCTCATCTTTTATCTGCTGACTCTTCTTGGCAACATGACCATTGTCTTGCTTTCAGC
TCTGGATTCCCGGCTGCACACACCAATGTATTTCTTTTTGGCAAACCTCTCATTCCTGGACATGTGTT
TCACCACAGGTTCCATCCCTCAGATGCTCTACAACCTTTGGGGTCCAGATAAGACCATCAGCTATGTG
GGTTGTGCCATCCAGCTGTACTTTGTCCTGGCCCTGGGAGGGGTGGAGTGTGTCCTCCTGGCTGTCAT
GGCATATGACCGCTATGCTGCAGTCTGCAAACCCCTGCACTACACCATCATCATGCACCCACGTCTCT
GTGGACAGCTGGCTTCAGTGGCATGGCTGAGTGGCTTTGGCAATTCTCTCATAATGGCACCCCAGACA
TTGATGCTACCCCGCTGTGGGCACAGACGAGTTGACCACTTTCTCTGTGAGATGCCAGCACTAATTGG
TATGGCCTGTGTAGACACCATGATGCTTGAGGCACTGGCTTTTGCCCTGGCAATCTTTATCATCCTGG
CACCACTCATCCTCATTCTCATTTCTTATGGTTACGTTGGAGGAACAGTGCTTAGGATCAAGTCAGCT
GCTGGGCGAAAGAAAGCCTCee CAACACTTGCAGCTCGCATCTAATTGTTGTCTCTCTCTTCTATGGTAC
AATTATATACATGTACCTCCAGCCAGCAAATACTTATTCCCAGGACCAGGGCAAGTTTCTTACCCTTT
TCTACACAATTGTCACTCCCAGTGTTAACCCCCTGATCTATACACTAAGAAACAAAGATGTTAAAGAG
GCCATGAAGAAGGTGCTAGGGAAGGGGAGTGCAGAAATATAGTAAGGG

| | | | |
|---|---|---|---|
| NOV42s, | | | |
| SNP13375126 of | | | |
| CG55358-03, | SEQ ID NO: 616 | | MW at 33991.2kD |
| Protein Sequence | SNP Pos: 233 | 307 aa SNP Change: Phe to Ser | |

TSSFEGFILVGFSDRPHLELIVFVVVLIFYLLTLLGNMTIVLLSALDSRLHTPMYFFLANLSFLDMCF
TTGSIPQMLYNLWGPDKTISYVGCAIQLYFVLALGGVECVLLAVMAYDRYAAVCKPLHYTIIMHPRLC
GQLASVAWLSGFGNSLIMAPQTLMLPRCGHRRVDHFLCEMPALIGMACVDTMMLEALAFALAIFIILA
PLILILISYGYVGGTVLRIKSAAGRKKASNTCSSHLIVVSLFYGTIIYMYLQPANTYSQDQGKFLTLF
YTIVTPSVNPLIYTLRNKDVKEAMKKVLGKGSAEI

A ClustalW comparison of the above protein sequences yields the following sequence alignment shown in Table 42B.

TABLE 42B
Comparison of the NOV42 protein sequences.

| | |
|---|---|
| NOV42a | MENDNTSSFEGFILVGFSDRPHLELIVFVVVLIFYLLTLLGNMTIVLLSALDSRLHTPMY |
| NOV42b | -----TSSFEGFILVGFSDRPHLELIVFVVVLIFYLLTLLGNMTIVLLSALDSRLHTPMY |
| NOV42c | RGSTMYKFFRGFILVGFSDRPHLELIVFVVVLIFYLLTLLGNMTIVLLSALDSRLHTPMY |
| NOV42d | ----------------------------RGSLIFYLLTLLGNMTIVLLSALDSRLHTPMY |
| NOV42e | ------------------------------------------------------------ |
| NOV42f | -ENDNTSSFEGFILVGFSDRPHLELIVFVVVLIFYLLTLLGNMTIVLLSALDSRLHTPMY |
| NOV42g | MENDNTSSFEGFILVGFSDRPHLELIVFVVVLIFYLLTLLGNMTIVLLSALDSRLHTPMY |
| NOV42h | MENDNTSSFEGFILVGFSDRPHLELIVFVVVLIFYLLTLLGNMTIVLLSALDSRLHTPMY |
| NOV42i | ------------------------------------------------------------ |
| NOV42j | ------------------------------------------------------------ |
| NOV42k | -ENDNTSSFEGFILVGFSDRPHLELIVFVVVLIFYLLTLLGNMTIVLLSALDSRLHTPMY |
| NOV42a | FFLANLSFLDMCFTTGSIPQMLYNLWGPDKTISYVGCAIQLYFVLALGGVECVLLAVMAY |
| NOV42b | FFLANLSFLDMCFTTGSIPQMLYNLWGPDKTISYVGCAIQLYFVLALGGVECVLLAVMAY |
| NOV42c | FFLANLSFLDMCFTTGSIPQMLYNLWGPDKTISYVGCAIQLYFVLALGGVECVLLAVMAY |
| NOV42d | FFLANLSFLDMCFTTGSIPQMLYNLWGPDKTISYVGCAIQLYFVLALGGVECVLLAVMAY |
| NOV42e | ----------------------------------RGSAIQLYFVLALGGVECVLLAVMAY |
| NOV42f | FFLANLSFLDMCFTTGSIPQMLYNLWGPDKTISYVGCAIQLYFVLALGGVECVLLAVMAY |
| NOV42g | FFLANLSFLDMCFTTGSIPQMLYNLWGPDKTISYVGCAIQLYFVLALGGVECVLLAVMAY |
| NOV42h | FFLANLSFLDMCFTTGSIPQMLYNLWGPDKTISYVGCAIQLYFVLALGGVECVLLAVMAY |
| NOV42i | ------------------------------------------------------------ |
| NOV42j | ------------------------------------------------------------ |
| NOV42k | FFLANLSFLDMCFTTGSIPQMLYNLWGPDKTISYVGCAIQLYFVLALGGVECVLLAVMAY |
| NOV42a | DRYAAVCKPLHYTIIMHPRLCGQLASVAWLSGFGNSLIMAPQTLMLPRCGHRRVDHFLCE |
| NOV42b | DRYAAVCKPLHYTIIMHPRLCGQLASVAWLSGFGNSLIMAPQTLMLPRCGHRRVDHFLCE |
| NOV42c | DRYAAVCKPLHYTIIMHPRLCGQLVSVAWLSGFGNSLIMAPQTLMLPRCGHRRVDHFLCE |
| NOV42d | DRYAAVCKPLHYTIIMHPRLCGQLVSVAWLSGFGNSLIMAPQTLMLPRCGHRRVDHFLCE |
| NOV42e | DRYAAVCKPLHYTIIMHPRLCGQLVSVAWLSGFGNSLIMAPQTLMLPRCGHRRVDHFLCE |
| NOV42f | DRYAAVCKPLHYTIIMHPRLCGQLASVAWLSGFGNSLIMAPQTLMLPRCGHRRVDHFLCE |
| NOV42g | DRYAAVCKPLHYTIIMHPRLCGQLASVAWLSGFGNSLIMAPQTLMLPRCGHRRVDHFLCE |
| NOV42h | DRYAAVCKPLHYTIIMHPRLCGQLASVAWLSGFGNSLIMAPQTLMLPRCGHRRVDHFLCE |
| NOV42i | --------------------------------MENDNTSSFEGFILVGFSDRP------- |
| NOV42j | ------------------------------------------TLMLPRCGHRRVDHFLCE |
| NOV42k | DRYAAVCKPLHYTIIMHPRLCGQLASVAWLSGFGNSLIMAPQTLMLPRCGHRRVDHFLCE |
| NOV42a | MPALIGMACVDTMMLEALAFALAIFIVLAPLILILISYGYVGGTVLRIKSAAGRKKAFNT |
| NOV42b | MPALIGMACVDTMMLEALAFALAIFIILAPLILILISYGYVGGTVLRIKSAAGRKKAFNT |
| NOV42c | MPALIGMACVDTMMLEALAFALAIFIILAPLILILISYGYVGGTVLRILEA--------- |
| NOV42d | MPALIGMACVDTMMLEALAFALAIFIILAPLILILISYGYVGGTVLRILEA--------- |
| NOV42e | MPALIGMACVDTMMLEALAFALAIFIILAPLILILISYGYVGGTVLRILEAKGEFQHT-- |
| NOV42f | MPALIGMACVDTMMLEALAFALAIFIILAPLILILISYGYVGGTVLRIKSAAGRKKAFNT |
| NOV42g | MPALIGMACVDTMMLEALAFALAIFIILAPLILILISYGYVGGTVLRIKSAAGRKKAFNT |
| NOV42h | MPALIGMACVDTMMLEALAFALAIFIILAPLILILISYGYVGGTVLRIKSAAGRKKAFNT |
| NOV42i | ------------------------------------------------------------ |
| NOV42j | MP---------------------------------------------------------- |
| NOV42k | MPALIGMACVDTMMLEALAFALAIFIILAPLILILISYGYVGGTVLRIKSAAGRKKAFNT |
| NOV42a | CSSHLIVVSLFYGTIIYMYLQPANTYSQDQGKFLTLFYTIVTPSVNPLIYTLRNKDVKEA |
| NOV42b | CSSHLIVVSLFYGTIIYMYLQPANTYSQDQGKFLTLFYTIVTPSVNPLIYTLRNKDVKEA |
| NOV42c | ------------------------------------------------------------ |
| NOV42d | ------------------------------------------------------------ |
| NOV42e | ------------------------------------------------------------ |
| NOV42f | CSSHLIVVSLFYGTIIYMYLQPANTYSQDQGKFLTLFYTIVTPSVNPLIYTLRNKDVKEA |
| NOV42g | CSSHLIVVSLFYGTIIYMYLQPANTYSQDQGKFLTLFYTIVTPSVNPLIYTLRNKDVKEA |
| NOV42h | CSSHLIVVSLFYGTIIYMYLQPANTYSQDQGKFLTLFYTIVTPSVNPLIYTLRNKDVKEA |
| NOV42i | ------------------------------------------------------------ |
| NOV42j | ------------------------------------------------------------ |
| NOV42k | CSSHLIVVSLFYGTIIYMYLQPANTYSQDQGKFLTLFYTIVTPSVNPLIYTLRNKDVKEA |
| NOV42a | MKKVLGKGSAEI |
| NOV42b | MKKVLGKGSAEI |
| NOV42c | ------------ |
| NOV42d | ------------ |
| NOV42e | ------------ |
| NOV42f | MKKVLGKGSAEI |
| NOV42g | MKKVLGKGSAEI |
| NOV42h | MKKVLGKGSAEI |
| NOV42i | ------------ |
| NOV42j | ------------ |
| NOV42k | MKKVLGKGSAEI |
| NOV42a | (SEQ ID NO: 580) |
| NOV42b | (SEQ ID NO: 582) |
| NOV42c | (SEQ ID NO: 584) |
| NOV42d | (SEQ ID NO: 586) |
| NOV42e | (SEQ ID NO: 588) |
| NOV42f | (SEQ ID NO: 590) |
| NOV42g | (SEQ ID NO: 592) |
| NOV42h | (SEQ ID NO: 594) |
| NOV42i | (SEQ ID NO: 596) |
| NOV42j | (SEQ ID NO: 598) |
| NOV42k | (SEQ ID NO: 600) |

Further analysis of the NOV42a protein yielded the following properties shown in Table 42C.

TABLE 42C
Protein Sequence Properties NOV42a

| | |
|---|---|
| SignalP analysis: | Cleavage site between residues 42 and 43 |
| PSORT II analysis: | |

| PSG: | a new signal peptide prediction method | | | |
|---|---|---|---|---|
| | N-region: | length 10; | pos.chg 0; | neg.chg 3 |
| | H-region: | length 8; | peak value | 0.00 |

| | PSG score: −4.40 |
|---|---|
| GvH: | von Heijne's method for signal seq. recognition |
| | GvH score (threshold: −2.1): −2.80 |
| | possible cleavage site: between 41 and 42 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood =−13.21 | Transmembrane 25-41 |
| | INTEGRAL | Likelihood = −7.11 | Transmembrane 103-119 |
| | INTEGRAL | Likelihood = −0.27 | Transmembrane 183-199 |
| | INTEGRAL | Likelihood =−14.97 | Transmembrane 200-216 |
| | INTEGRAL | Likelihood = −1.12 | Transmembrane 244-260 |

| | |
|---|---|
| PERIPHERAL | Likelihood = 1.80 (at 273) |

| | ALOM score: −14.97 (number of TMSs: 5) |
|---|---|
| MTOP: | Prediction of membrane topology (Hartmann et al.) |
| | Center position for calculation: 32 |
| | Charge difference: 2.0 C(0.5) − N(−1.5) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 6.56 |
| Hyd Moment(95): | 3.47 | G content: | 0 |
| D/E content: | 2 | S/T content: | 0 |
| Score: −7.29 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 6.7%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
22.2%: vacuolar
11.1%: vesicles of secretory system
11.1%: Golgi

>> prediction for CG55358-04 is end (k = 9)

A search of the NOV42a protein against the Geneseq database, a proprietary database that contains sequences published in patents and patent publication, yielded several homologous proteins shown in Table 42D.

TABLE 42D
Geneseq Results for NOV42a

| | | NOV42a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE10685 | G-protein coupled receptor 4a | 1 . . . 312 | 311/312 (99%) | e−180 |
| | (GPCR4a) - Unidentified, 312 aa. | 1 . . . 312 | 312/312 (99%) | |
| | [WO200164879-A2, 07-SEP- | | | |
| | 2001] | | | |
| AAG72062 | Human olfactory receptor | 1 . . . 312 | 310/312 (99%) | e−179 |
| | polypeptide, SEQ ID NO: 1743 - | 3 . . . 314 | 312/312 (99%) | |
| | Homo sapiens, 334 aa. | | | |
| | [WO200127158-A2, 19-APR- | | | |
| | 2001] | | | |
| AAE10686 | G-protein coupled receptor 4b | 6 . . . 312 | 306/307 (99%) | e−177 |
| | (GPCR4b) - Unidentified, 307 aa. | 1 . . . 307 | 307/307 (99%) | |
| | [WO200164879-A2, 07-SEP- | | | |
| | 2001] | | | |
| ABP95745 | Human GPCR polypeptide SEQ | 43 . . . 312 | 269/270 (99%) | e−155 |
| | ID NO 300 - Homo sapiens, 270 | 1 . . . 270 | 270/270 (99%) | |
| | aa. [WO200216548-A2, 28-FEB- | | | |
| | 2002] | | | |
| AAG71998 | Human olfactory receptor | 43 . . . 312 | 269/270 (99%) | e−155 |
| | polypeptide, SEQ ID NO: 1679 - | 1 . . . 270 | 270/270 (99%) | |
| | Homo sapiens, 270 aa. | | | |
| | [WO200127158-A2, 19-APR- | | | |
| | 2001] | | | |

In a BLAST search of public sequence databases, the NOV42a protein was found to have homology to the proteins shown in the BLASTP data in Table 42E.

TABLE 42E
Public BLASTP Results for NOV42a

| | | NOV42a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC88325 | Sequence 11 from Patent | 1 . . . 312 | 311/312 (99%) | e−179 |
| | WO0164879 - Homo sapiens | 1 . . . 312 | 312/312 (99%) | |
| | (Human), 312 aa. | | | |
| Q8NHA6 | Seven transmembrane helix | 12 . . . 312 | 300/301 (99%) | e−173 |
| | receptor - Homo sapiens | 18 . . . 318 | 301/301 (99%) | |
| | (Human), 318 aa. | | | |
| Q8VFH0 | Olfactory receptor MOR256-12 - | 1 . . . 312 | 295/312 (94%) | e−172 |
| | Mus musculus (Mouse), 317 | 6 . . . 317 | 304/312 (96%) | |
| | aa. | | | |
| Q8VG14 | Olfactory receptor MOR256-8 - | 1 . . . 307 | 212/307 (69%) | e−125 |
| | Mus musculus (Mouse), 308 aa. | 1 . . . 307 | 251/307 (81%) | |
| Q8VFG9 | Olfactory receptor MOR256-13 - | 5 . . . 305 | 205/301 (68%) | e−120 |
| | Mus musculus (Mouse), 314 | 11 . . . 311 | 247/301 (81%) | |
| | aa. | | | |

PFam analysis indicates that the NOV42a protein contains the domains shown in the Table 42F.

TABLE 42F
Domain Analysis of NOV42a

| Pfam | NOV42a | Identities/Similarities | Expect |
|---|---|---|---|
| Domain | Match Region | for the Matched Region | Value |
| 7tm_1 | 41 . . . 290 | 57/276 (21%) | 3.2e−41 |
| | | 177/276 (64%) | |

### Example 43

The NOV43 clone was analyzed, and the nucleotide and encoded polypeptide sequences are shown in Table 43A.

TABLE 43A

NOV43 Sequence Analysis

| | | |
|---|---|---|
| NOV43a, CG55604-04 | SEQ ID NO: 617 | 968 bp |
| DNA Sequence | ORF Start: ATG at 8 | ORF Stop: TGA at 950 |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATAT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATGGAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43a, CG55604-04 | | | |
| Protein Sequence | SEQ ID NO: 618 | 314 aa | MW at 35728.1kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43b, GG55604-01 | SEQ ID NO: 619 | 840 bp |
| DNA Sequence | ORF Start: ATG at 42 | ORF Stop: TGA at 810 |

TCATCATTCTCATCTTCCTGGATTCTCGCCTTCACACTCCCATGTATTTTTTTTCTTAGAAACTCTCC
TTTGCAGATCTCTGTTTCTCTACTAGCATTGTCCCTCAAGTGTTGGTTCACTTCTTGGTAAAGAGGAA
AACCATTTCTTTTTATGGGTGTATGACACAGATAATTGTCTTTCTTCTGGTTGGGTGTACAGAGTGTG
CGCTGCTGGCAGTGATGTCCTATGACCGGTATGTGGCTGTCTGCAAGCCCCTGTACTACTCTACCATC
ATGACACAACGGGTGTGTCTCTGGCTGTCCTTCAGGTCCTGGGCCAGTGGGGCACTAGTGTCTTTAGT
AGATACCAGCTTTACTTTCCATCTTCCCTACTGGGGACAGAATATAATCAATCACTACTTTTGTGAAC
CTCCTGCCCTCCTGAAGCTGGCTTCCATAGACACTTACAGCACAGAAATGGCCATCTTTTCAATGGGC
GTGGTAATCCTCCTGGCCCCTGTCTCCCTGATTCTTGGTTCTTATTGGAATATTATCTCCACTGTTAT
CCAGATGCAGTCTGGGGAAGGGAGACTCAAGGCTTTTTCCACCTGTGGCTCCCATCTTATTGTTGTTG
TCCTCTTCTATGGGTCAGGAATATTCACCTACATGCGACCAAACTCCAAGACTACAAAAGAACTGGAT
AAAATGATATCTGTGTTCTATACAGCGGTGACTCCAATGTTGAACCCCATAATTTATAGCTTGAGGAA
CAAAGATGTCAAAGGGGCTCTCAGGAAACTAGTTGGGAGAAAGTGCTTCTCTCATAGGCAGTGACCTC
TGAGTCTGACTTTTAGAGCTATGG

| | | | |
|---|---|---|---|
| NOV43b, CG55604-01 | | | |
| Protein Sequence | SEQ ID NO: 620 | 256 aa | MW at 29023.1kD |

MYFFLRNLSFADLCFSTSIVPQVLVHFLVKRTISFYGCMTQIIVFLLVGCTECALLAVMSYDRYAVAV
CKPLYYSTIMTQRVCLWLSFRSWASGALVSLVDTSFTFHLPWGQNIINHYFCEPPALLKLASIADTYS
TEMAIFSMGVVILLAPVSILGSYWNIISTVIQMQSGEGRLKAFSTCGSHLIVVVLFYGSGIFATYMRP
NSKTTKELDKMISVFYTAVTPMLNPIIYSLRNKDVKGALRKLVGRKCFSHRQ

| | | |
|---|---|---|
| NOV43c, GG55604-02 | SEQ ID NO: 621 | 944 bp |
| DNA Sequence | ORF Start: ATG at 31 | ORF Stop: TGA at 973 |

TGCCAAACAGGTAAACAGGCAAAAATATCAATGGGAGAAGAAAACCAAACCTTTGTGTCCAAGTTTAT
CTTCCTGGGTCTTTCACAGGACTTGCAGACCCAGATCCTGCTATTTATCCTTTTCCTCATCATTTATC
TGCTGACCGTGCTTGGAAACCAGCTCATCATCATTCTCATCTTCCTGGATTCTCGCCTTCACACTCCC
ATGTATTTTTTTCTTAGAAATATCTCCTTTGCAGATCTCTGTTTCTCTACTAGCATTGTCCCTCAAGT
GTTGGTTCACTTCTTGGTAAAGAGGAAAACCATTTCTTTTTATGGGTGTATGACACAGATAATTGTCT
TTCTTCTGGTTGGGTGTACAGAGTGTGCGCTGCTGGCAGTGATGTCCTATGACCGGTATGTGGCTGTC
TGCAAGCCCCTGTACTACTCTACCATCATGACACAACGGGTGTGTCTCTGGCTGTCCTTCAGGTCCTG
GGCCAGTGGGGCACTAGTGTCTTTAGTAGATACCAGCTTTACTTTCCATCTTCCCTACTGGGGACAGA
ATATAATCAATCACTACTTTTGTGAACCTCCTGCCCTCCTGAAGCTGGCTTCCATAGACACTTACAGC
ACAGAAATGGCCATCTTTTCAATGGGCGTGGTAATCCTCCTGGCCCCTGTCTCCCTGATTCTTGGTTC
TTATTGGAATATTATCTCCACTGTTATCCAGATGCAGTCTGGGGAAGGGAGACTCAAGGCTTTTTCCA
CCTGTGGCTGGGATCTTATTGTTGTTGTCCTCTTCTATGGGTCAGGAATATTCACCTACATGCGACCA
AACTCCAAGACTACAAAAGAACTGGATAAAATGATATCTGTGTTCTATACAGCGGTGACTCCAATGTT
GAACCCCATAATTTATAGCTTGAGGAACAAAGATGTCAAAGGGGCTCTCAGGAAACTAGTTGGGAGAA
AGTGCTTCTCTCATAGGCATGACCTCTGAGTCTGACTTTTA

| | | | |
|---|---|---|---|
| NOV43c, GG55604-02 | | | |
| Protein Sequence | SEQ ID NO: 622 | 314 aa | MW at 35714.lkD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43d, CG55604-03 | SEQ ID NO: 623 | 960 bp |
| DNA Sequence | ORF Start: ATG at 1 | ORF Stop: TGA at 943 |

ATGGGAGAAGAAAACCAAACCTTTGTGTCCAAGTTTATCTTCCTGGGTCTTTCACAGGACTTGCAGAC
CCAGATCCTGCTATTTATCCTTTTCCTCATCATTTTATCTGTGACCGTGCTTGGAAACCAGCTCATCA
TCATTCTCATCTTCCTGGATTCTCGCCTTCACACTCCCATGTATTTTTTTCTTAGAAATATCTCCTTT
GCAGATCTCTGTTTCTCTACTAGCATTGTCCCTCAAGTGTTGGTTCACTTCTTGGTAAAGAGGAAAAC
CATTTCTTTTTATGGGTGTATGACACAGATAATTGTCTTTCTTCTGGTTGGGTGTACAGAGTGTGCGC
TGCTGGCAGTGATGTCCTATGACCGGTATGTGGCTGTCTGCAAGCCCCTGTACTACTCTACCATCATG
ACACAACGGGTGTGTCTCTGGCTGTCCTTCAGGTCCTGGGCCAGTGGGGCACTAGTGTCTTTAGTAGA
TACCAGCTTTACTTTCCATCTTCCCTACTGGGGACAGAATATAATCAATCACTACTTTTGTGAACCTC
CTGCCCTCCTGAAGCTGGCTTCCATAGACACTTACAGCACAGAAATGGCCATCTTTTCAATGGGCGTG
GTAATCCTCCTGGCCCCTATCTCCCTGATTCTTGGTTCTTATTGGAATATTATCTCCACTGTTATCCA
GATGCAGTCTGGGGAAGGGAGACTCAAGGCTTTTTCCACCTGTGGCTCCCATCTTATTGTTGTTGTCC
TCTTCTATGGGTCAGGAATATTCACCTACATGCGACCAAACTCCAAGACTACAAAAGAACTGGATAAA
ATGATATCTGTGTTCTATACAGCGGTGACTCCAATGTTGAACCCCATAATTTATAGCTTGAGGAACAA
AGATGTCAAAGGGGCTCTCAGGAAACTAGTTGGGAGAAAGTGCTTCTCTCATAGGCAGTGACCTCTGA
GTCTGACT

| | | | |
|---|---|---|---|
| NOV43d, CG55604-03 | | | |
| Protein Sequence | SEQ ID NO: 624 | 314 aa | MW at 35728.1kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43e, GG55604-05 | SEQ ID NO: 625 | 995 bp |
| DNA Sequence | ORF Start: ATG at 31 | ORF Stop: TGA at 973 |

TGCCAAACAGGTAAACAGGCAAAAATATCAATGGGAGAAGAAAACCAAACCTTTGTGTCCAAGTTTAT
CTTCCTGGGTCTTTCACAGGACTTGCAGACCCAGATCCTGCTATTTATCCTTTTCCTCATCATTTATC
TGCTGACCGTGCTTGGAAACCAGCTCATCATCATTCTCATCTTCCTGGATTCTCGCCTTCACACTCCC
ATGTATTTTTTTCTTAGAAATCTCTCCTTTGCAGATCTCTGTTTCTCTACTAGCATTGTCCCTCAAGT
GTTGGTTCACTTCTTGGTAAAGAGGAAAACCATTTCTTTTTATGGGTGTATGACACAGATAATTGTCT
TTCTTCTGGTTGGGTGTACAGAGTGTGCGCTGCTGGCCGTGATGTCCTATGACCGGTATGTGGCTGTC
TGCAAGCCCCTGTACTACTCTACCATCATGACACAACGGGTGTGTCTCTGGCTGTCCTTCAGGTCCTG
GGCCAGTGGGGCACTAGTGTCTTTAGTAGATACCAGCTTTACTTTCCATCTTCCCTACTGGGGACAGA
ATATAATCAATCACTACTTTTGTGAACCTCCTGCCCTCCTGAAGCTGGCTTCCATAGACACTTACAGC
ACAGAAATGGCCATCTTTTCAATGGGCGTGGTAATCCTCCTGGCCCCTATCTCCCTGATTCTTGGTTC
TTATTGGAATATTATCTCCACTGTTATCCAGATGCAGTCTGGGGAAGGGAGACTCAAGGCTTTTTCCA
CCTGTGGCTCCCATCTTATTGTTGTTGTCCTCTTCTATGGGTCAGGAATATTCACCTACATGCGACCA
AACTCCAAGACTACAAAAGAACTGGATAAAATGATATCTGTGTTCTATACAGCGGTGACTCCAATGTT
GAACCCCATAATTTATAGCTTGAGGAACAAAGATGTCAAAGGGGCTCTCAGGAAACTAGTTGGGAGAA
AGTGCTTCTCTCATAGGCAGTGACCTCTGAGTCTGACTTTTAA

| | | | |
|---|---|---|---|
| NOV43e, CG55604-05 | | | |
| Protein Sequence | SEQ ID NO: 626 | 314 aa | MW at 35728.1kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43f, CG55604-06 | SEQ ID NO: 627 | 946 bp |
| DNA Sequence | ORF Start: ATG at 2 | ORF Stop: TGA at 944 |

TATGGGAGAAGAAAACCAAACCTTTGTGTCCAAGTTTATCTTCCTGGGTCTTTCACAGGACTTGCAGA
CCCAGATCCTGCTATTTATCCTTTTCCTCATCATTTATCTGCTGACCGTGCTTGGAAACCAGCTCATC
ATCATTCTCATCTTCCTGGATTCTCGCCTTCACACTCCCATGTATTTTTTTCTTAGAAATATCTCCTT
TGCAGATCTCTGTTTTCTCTACTAGCATTGTCCCTCAAGTGTTGGTTCACTTCTTGGTAAAGAGGAAA
CCATTTCTTTTTATGGGTGTATGACACAGATAATTGTCTTTCTTCTGGTTGGGTGTACAGAGTGTGCG
CTGCTGGCATTGATGTCCTATGACCGGTATGTGGCTGTCTGCAAGCCCCTGTACTACTCTACCATCAT
GACACAACGGGTGTGTCTCTGGCTGTCCTTCAGGTCCTCGGCCAGTGGGGCACTAGTGTCTTTAGTAG
ATACCAGCTTTACTTTCCATCTTCCCTACTGGGGACAGAATATAATCAATCACTACTTTTGTGAACCT
CCTGCCCTCCTGAAGCTGGCTTCCATAGACACTTACAGCACAGAAATGGCCATCTTTTCAATGGGCGT
GGTAATCCTCCTGGCCCCTGTCTCCCTGATTCTTGGTTCTTATTGGAATATTATCTCCACTGTTATCC
AGATGCAGTCTGGGGAAGGGAGACTCAAGGCTTTTTCCACCTGTGGCTCCCATCTTATTGTTGTTGTC
CTCTTCTATGGGTCAGGAATATTCACCTACATGCGACCAAACTCCAAGACTACAAAAGAACTGGATAA
AATGATATCTGTGTTCTATACAGCGGTGACTCCAATGTTGAACCCCATAATTTATAGCTTGAGGAACA
AAGATGTCAAAGGGGTCTCAGGAAACTAGTTGGGAGAAAGTGCTTCTCTCATAGGCAGTGA

| | | | |
|---|---|---|---|
| NOV43f, CG55604-06 | | | |
| Protein Sequence | SEQ ID NO: 628 | 314 aa | MW at 35614.9kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43g, CG55604-07 | SEQ ID NO: 629 | 962 bp |
| DNA Sequence | ORF Start: ATG at 2 | ORF Stop: TGA at 944 |

TATGGGAGAAGAAAACCAAACCTTTGTGTCCAAGTTTATCTTCCTGGGTCTTTCACAGGACTTGCAGA
CCCAGATCCTGCTATTTATCCTTTTCCTCATCATTTATCTGCTGACCGTGCTTGGAAACCAGCTCATC
ATCATTCTCATCTTCCTGGATTCTCGCCTTCACACTCCCATGTATTTTTTTCTTAGAAATATCTCCTT
TGCAGATCTCTGTTTTCTCTACTAGCATTGTCCCTCAAGTGTTGGTTCACTTCTTGGTAAAGAGGAAA
CCATTTCTTTTTATGGGTGTATGACACAGATAATTGTCTTTCTTCTGGTTGGGTGTACAGAGTGTGCG
CTGCTGGCATTGATGTCCTATGACCGGTATGTGGCTGTCTGCAAGCCCCTGTACTACTCTACCATCAT
GACACAACGGGTGTGTCTCTGGCTGTCCTTCAGGTCCTCGGCCAGTGGGGCACTAGTGTCTTTAGTAG
ATACCAGCTTTACTTTCCATCTTCCCTACTGGGGACAGAATATAATCAATCACTACTTTTGTGAACCT
CCTGCCCTCCTGAAGCTGGCTTCCATAGACACTTACAGCACAGAAATGGCCATCTTTTCAATGGGCGT
GGTAATCCTCCTGGCCCCTGTCTCCCTGATTCTTGGTTCTTATTGGAATATTATCTCCACTGTTATCC
AGATGCAGTCTGGGGAAGGGAGACTCAAGGCTTTTTCCACCTGTGGCTCCCATCTTATTGTTGTTGTC
CTCTTCTATGGGTCAGGAATATTCACCTACATGCGACCAAACTCCAAGACTACAAAAGAACTGGATAA
AATGATATCTGTGTTCTATACAGCGGTGACTCCAATGTTGAACCCCATAATTTATAGCTTGAGGAACA
AAGATGTCAAAGGGGCTCTCAGGAAACTAGTTGGGAGAAAGTGCTTCTCTCATAGGCAGTGACCTCTG
AGTCTGACTA

| | | | |
|---|---|---|---|
| NOV43g, CG55604-07 | | | |
| Protein Sequence | SEQ ID NO: 630 | 314 aa | MW at 35728.1kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43h, | | |
| SNP13019742 of | SEQ ID NO: 631 | 968 bp |
| CG55604-04, | ORF Start: ATG at 8 | ORF Stop: TGA at 950 |
| DNA Sequence | SNP Pos: 203 | SNP Change: A to C |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43h, | | | |
| SNP13019742 of | | | |
| CG55604-04, | SEQ ID NO: 632 | | |
| Protein Sequence | SNP Pos: 66 | 314 aa | MW at 35728.1kD |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43i, | | |
| SNP13373776 of | SEQ ID NO: 633 | 968 bp |
| CG55604-04, | ORF Start: ATG at 8 | ORF Stop: TGA at 950 |
| DNA Sequence | SNP Pos: 355 | SNP Change: A to C |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43i, | | | |
| SNP13373776 of | | | |
| CG55604-04, | SEQ ID NO: 634 | | MW at 35728.1kD |
| Protein Sequence | SNP Pos: 116 | 314 aa | SNP Change: Ala to Ala |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43j, | | |
| SNP13373777 of | SEQ ID NO: 635 | 968 bp |
| CG55604-04, | ORF Start: ATG at 8 | ORF Stop: TGA at 950 |
| DNA Sequence | SNP Pos: 372 | SNP Change: G to A |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCAGGTTGTGGCTGTCTGCAAGCCCCTGTACTACTCTAC
CATCATGACACAACGGGTGTGTCTCTGGCTGTCCTTCAGGTCCTGGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43j, | | | |
| SNP13373777 of | | | |
| CG55604-04, | SEQ ID NO: 636 | | MW at 35700.0kD |
| Protein Sequence | SNP Pos: 122 | 314 aa | SNP Change: Arg to Gln |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDQYVAVCKPLYYSTIM
TQRVCLWLSFRSWASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43k, | | |
| SNP13373778 of | SEQ ID NO: 637 | 968 bp |
| CG55604-04, | ORF Start: ATG at 8 | ORF Stop: TGA at 950 |
| DNA Sequence | SNP Pos: 422 | SNP Change: C to T |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAATGGGTGTGTCTCTGGCTGTCCTTCAGGTCCTGGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43k, | | | |
| SNP13373778 of | | | |
| CG55604-04, | SEQ ID NO: 638 | | MW at 35758.1kD |
| Protein Sequence | SNP Pos: 139 | 314 aa | SNP Change: Arg to Trp |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQWVCLWLSFRSWASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43l, | | |
| SNP13373838 of | SEQ ID NO: 639 | 968 bp |
| CG55604-04, | ORF Start: ATG at 8 | ORF Stop: TGA at 950 |
| DNA Sequence | SNP Pos: 453 | SNP Change: G to C |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTCGGCCAGTGGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43l, | | | |
| SNP13373838 of | | | |
| CG55604-04, | SEQ ID NO: 640 | | MW at 35628.9kD |
| Protein Sequence | SNP Pos: 149 | 314 aa | SNP Change: Trp to Ser |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSSASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43m, | | |
| SNP13373837 of | SEQ ID NO: 641 | 968 bp |
| CG55604-04, | ORF Start: ATG at 8 | ORF Stop: TGA at 950 |
| DNA Sequence | SNP Pos: 461 | SNP Change: G to A |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACAGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43m, | | | |
| SNP13373837 of | | | |
| CG55604-04, | SEQ ID NO: 642 | | MW at 35827.2kD |
| Protein Sequence | SNP Pos: 152 | 314 aa | SNP Change: Gly to Arg |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASRALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43n, | | |
| SN1P13373836 of | SEQ ID NO: 643 | 968 bp |
| CG55604-04, | ORF Start: ATG at 8 | ORF Stop: TGA at 950 |
| DNA Sequence | SNP Pos: 587 | SNP Change: A to G |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43n, | | | |
| SNP13373836 of | | | |
| CG55604-04, | SEQ ID NO: 644 | | MW at 35698.1kD |
| Protein Sequence | SNP Pos: 194 | 314 aa | SNP Change: Ser to Gly |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYGTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43o, | | |
| SNP13373780 of | SEQ ID NO: 645 | 968 bp |
| CG55604-04, | ORF Start: ATG at 8 | ORF Stop: TGA at 950 |
| DNA Sequence | SNP Pos: 615 | SNP Change: G to A |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGACGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43o, | | | |
| SNP13373780 of | | | |
| CG55604-04, | SEQ ID NO: 646 | | MW at 35786.1kD |
| Protein Sequence | SNP Pos: 203 | 314 aa | SNP Change: Gly to Asp |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMDV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43p, | | |
| SNP13373781 of | SEQ ID NO: 647 | 968 bp |
| CG55604-04, | ORF Start: ATG at 8 | ORF Stop: TGA at 950 |
| DNA Sequence | SNP Pos: 638 | SNP Change: A to G |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTGTCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43p, | | | |
| SNP13373781 of | | | |
| CG55604-04, | SEQ ID NO: 648 | | MW at 35714.1kD |
| Protein Sequence | SNP Pos: 211 | 314 aa | SNP Change: Ile to Val |

MGEENQTFVSKFIFLGLSQDLQTQILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPVSLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43q, | | |
| SNP13373782 of | SEQ ID NO: 649 | 968 bp |
| CG55604-04, | ORF Start: ATG at 8 | ORF Stop: TGA at 950 |
| DNA Sequence | SNP Pos: 679 | SNP Change: T to C |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACCG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43q, | | | |
| SNP13373782 of | | | |
| CG55604-04, | SEQ ID NO: 650 | | MW at 35728.1kD |
| Protein Sequence | SNP Pos: 224 | 314 aa | SNP Change: Thr to Thr |

MGEENQTFVSKFIFLGLSQDLQTQILLFILFLIIYLLTVLGNOLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGONIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRQ

| | | |
|---|---|---|
| NOV43r, | | |
| SNP13373783 of | SEQ ID NO: 651 | 1968 bp |
| CG55604-04, | ORF Start: ATG at 8 | ORF Stop: TGA at 950 |
| DNA Sequence | SNP Pos: 844 | SNP Change: A to G |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACGGCGGTGACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43r, | | | |
| SNP13373783 of | | | |
| CG55604-04, | SEQ ID NO: 652 | | MW at 35728.1kD |
| Protein Sequence | SNP Pos: 279 | 314 aa | SNP Change: Thr to Thr |

MGEENQTFVSKFIFLGLSQDLQTQILLFILFLIIYLLTVLGNOLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRO

| | | |
|---|---|---|
| NOV43s, | | |
| SNP13373833 of | SEQ ID NO: 653 | 968 bp |
| CG55604-04, | ORF Start: ATG at 8 | ORF Stop: TGA at 950 |
| DNA Sequence | SNP Pos: 850 | SNP Change: G to A |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTAACTCCAATGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43s, | | | |
| SNP13373833 of | | | |
| CG55604-04, | SEQ ID NO: 654 | | MW at 35728.1kD |
| Protein Sequence | SNP Pos: 281 | 314 aa | SNP Change: Val to Val |

MGEENQTFVSKFIFLGLSQDLQTQILLFILFLIIYLLTVLGNOLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPYWGQNIINHYFCEPPALLKLASIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPMLNPIIYSLRNKDVKGALRKLVGRKCFSHRO

| | | |
|---|---|---|
| NOV43t, | | |
| SNP13373784 of | SEQ ID NO: 655 | 968 bp |
| CG55604-04, | ORF Start: ATG at 8 | ORF Stop: TGA at 950 |
| DNA Sequence | SNP Pos: 857 | SNP Change: A to G |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCAGTGTTGAACCCCATAATTTATAGCTTGA
GGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43t, | | | |
| SNP13373784 of | | | |
| CG55604-04, | SEQ ID NO: 656 | | MW at 35696.0kD |
| Protein Sequence | SNP Pos: 284 | 314 aa | SNP Change: Met to Val |

MGEENQTFVSKFIFLGLSQDLQTOILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPWGQNIINHYFCEPPALLKLASAIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPVLNPIIYSLRNKDVKGALRKLVGRKCFSHRQ

| | | |
|---|---|---|
| NOV43u, | | |
| SNP13373832 of | SEQ ID NO: 657 | 968 bp |
| CG55604-04, | ORF Start: ATG at 8 | ORF Stop: TGA at 950 |
| DNA Sequence | SNP Pos: 885 | SNP Change: G to A |

TATATCAATGGGAGAAGAAAACCAAACCTTTGTGTCCAAGTTTATCTTCCTGGGTCTTTCACAGGACT
TGCAGACCCAGATCCTGCTATTTATCCTTTTCCTCATCATTTATCTGCTGACCGTGCTTGGAAACCAG
CTCATCATCATTCTCATCTTCCTGGATTCTCGCCTTCACACTCCCATGTATTTTTTTCTTAGAAATCT
CTCCTTTGCAGATCTCTGTTTCTCTACTAGCATTGTCCCTCAAGTGTTGGTTCACTTCTTGGTAAAGA
GGAAAACCATTTCTTTTTATGGGTGTATGACACAGATAATTGTCTTTCTTCTGGTTGGGTGTACAGAG
TGTGCGCTGCTGGCAGTGATGTCCTATGACCGGTATGTGGCTGTCTGCAAGCCCCTGTACTACTCTAC
CATCATGACACAACGGGTGTGTCTCTGGCTGTCCTTCAGGTCCTGGGCCAGTAGGGCACTAGTGTCTT
TAGTAGATACCAGCTTTACTTTCCATCTTCCCTACTGGGGACAGAATATAATCAATCACTACTTTTGT
GAACCTCCTGCCCTCCTGAAGCTGGCTTCCATAGACACTTACGGCACAGAAATGGCCATCTTTTCAAT
GGGCGTGGTAATCCTCCTGGCCCCTATCTCCCTGATTCTTGGTTCTTATTGGAATATTATCTCCACTG
TTATCCAGATGCAGTCTGGGGAAGGGAGACTCAAGGCTTTTTCCACCTGTGGCTCCCATCTTATTGTT
GTTGTCCTCTTCTATGGGTCAGGAATATTCACCTACATGCGACCAAACTCCAAGACTACAAAAGAACT
GGATAAAATGATATCTGTGTTCTATACAGCGGTGACTCCCGTGTTGAACCCCATAATTTATAGCTTGA
AGAACAAAGATGTCAAAGGGGCTCTCAGGAAACTAGTTGGGAGAAAGTGCTTCTCTCATAGGCAGTGA
CCTCTGAGTCTGACTA

| | | | |
|---|---|---|---|
| NOV43u, | | | |
| SNP13373832 of | | | |
| CG55604-04, | SEQ ID NO: 658 | | MW at 35700.1kD |
| Protein Sequence | SNP Pos: 293 | 314 aa | SNP Change: Arg to Lys |

MGEENQTFVSKFIFLGLSQDLQTOILLFILFLIIYLLTVLGNQLIIILIFLDSRLHTPMYFFLRNISF
ADLCFSTSIVPQVLVHFLVKRKTISFYGCMTQIIVFLLVGCTECALLAVMSYDRYVAVCKPLYYSTIM
TQRVCLWLSFRSWASGALVSLVDTSFTFHLPWGQNIINHYFCEPPALLKLASAIDTYSTEMAIFSMGV
VILLAPISLILGSYWNIISTVIQMQSGEGRLKAFSTCGSHLIVVVLFYGSGIFTYMRPNSKTTKELDK
MISVFYTAVTPVLNPIIYSLKNKDVKGALRKLVGRKCFSHRQ

A ClustalW comparison of the above protein sequences yields the following sequence alignment shown in Table 43B.

TABLE 43B

Comparison of the NOV43 protein sequences.

| | |
|---|---|
| NOV43a | MGEENQTFVSKFIFLGLSQDLQTQILLFILFLIIYLLTVLGNQLIIILIFLDSRLHTPMY |
| NOV43b | ------------------------------------------------------------ |
| NOV43c | MGEENQTFVSKFIFLGLSQDLQTQILLFILFLIIYLLTVLGNQLIIILIFLDSRLHTPMY |
| NOV43d | MGEENQTFVSKFIFLGLSQDLQTQILLFILFLIIYLLTVLGNQLIIILIFLDSRLHTPMY |
| NOV43e | MGEENQTFVSKFIFLGLSQDLQTQILLFILFLIIYLLTVLGNQLIIILIFLDSRLHTPMY |
| NOV43f | MGEENQTFVSKFIFLGLSQDLQTQILLFILFLIIYLLTVLGNQLIIILIFLDSRLHTPMY |
| NOV43g | MGEENQTFVSKFIFLGLSQDLQTQILLFILFLIIYLLTVLGNQLIIILIFLDSRLHTPMY |
| NOV43a | FFLRNISFADLCFSTSIVPQVLVHFLVKRKTISFYGCMTQIIVFLLVGCTECALLAVMSY |
| NOV43b | FFLRNISFADLCFSTSIVPQVLVHFLVKRKTISFYGCMTQIIVFLLVGCTECALLAVMSY |
| NOV43c | FFLRNISFADLCFSTSIVPQVLVHFLVKRKTISFYGCMTQIIVFLLVGCTECALLAVMSY |
| NOV43d | FFLRNISFADLCFSTSIVPQVLVHFLVKRKTISFYGCMTQIIVFLLVGCTECALLAVMSY |
| NOV43e | FFLRNISFADLCFSTSIVPQVLVHFLVKRKTISFYGCMTQIIVFLLVGCTECALLAVMSY |
| NOV43f | FFLRNISFADLCFSTSIVPQVLVHFLVKRKTISFYGCMTQIIVFLLVGCTECALLAVMSY |
| NOV43g | FFLRNISFADLCFSTSIVPQVLVHFLVKRKTISFYGCMTQIIVFLLVGCTECALLAVMSY |
| NOV43a | DRYVAVCKPLYYSTIMTQRVCLWLSFRSWASGALVSLVDTSFTFHLPYWGQNIINHYFCE |
| NOV43b | DRYVAVCKPLYYSTIMTQRVCLWLSFRSWASGALVSLVDTSFTFHLPYWGQNIINHYFCE |
| NOV43c | DRYVAVCKPLYYSTIMTQRVCLWLSFRSWASGALVSLVDTSFTFHLPYWGQNIINHYFCE |
| NOV43d | DRYVAVCKPLYYSTIMTQRVCLWLSFRSWASGALVSLVDTSFTFHLPYWGQNIINHYFCE |
| NOV43e | DRYVAVCKPLYYSTIMTQRVCLWLSFRSWASGALVSLVDTSFTFHLPYWGQNIINHYFCE |
| NOV43f | DRYVAVCKPLYYSTIMTQRVCLWLSFRSWASGALVSLVDTSFTFHLPYWGQNIINHYFCE |
| NOV43g | DRYVAVCKPLYYSTIMTQRVCLWLSFRSWASGALVSLVDTSFTFHLPYWGQNIINHYFCE |
| NOV43a | PPALLKLASIDTYSTEMAIFSMGVVILLAPVSLILGSYWNIISTVIQMQSGEGRLKAFST |
| NOV43b | PPALLKLASIDTYSTEMAIFSMGVVILLAPVSLILGSYWNIISTVIQMQSGEGRLKAFST |
| NOV43c | PPALLKLASIDTYSTEMAIFSMGVVILLAPVSLILGSYWNIISTVIQMQSGEGRLKAFST |
| NOV43d | PPALLKLASIDTYSTEMAIFSMGVVILLAPVSLILGSYWNIISTVIQMQSGEGRLKAFST |
| NOV43e | PPALLKLASIDTYSTEMAIFSMGVVILLAPVSLILGSYWNIISTVIQMQSGEGRLKAFST |
| NOV43f | PPALLKLASIDTYSTEMAIFSMGVVILLAPVSLILGSYWNIISTVIQMQSGEGRLKAFST |
| NOV43g | PPALLKLASIDTYSTEMAIFSMGVVILLAPVSLILGSYWNIISTVIQMQSGEGRLKAFST |
| NOV43a | CGSHLIVVVLFYGSGIFTYMRPNSKTTKELDKMISVFYTAVTPMLNPIIYSLRNKDVKGA |
| NOV43b | CGSHLIVVVLFYGSGIFTYMRPNSKTTKELDKMISVFYTAVTPMLNPIIYSLRNKDVKGA |
| NOV43c | CGSHLIVVVLFYGSGIFTYMRPNSKTTKELDKMISVFYTAVTPMLNPIIYSLRNKDVKGA |
| NOV43d | CGSHLIVVVLFYGSGIFTYMRPNSKTTKELDKMISVFYTAVTPMLNPIIYSLRNKDVKGA |
| NOV43e | CGSHLIVVVLFYGSGIFTYMRPNSKTTKELDKMISVFYTAVTPMLNPIIYSLRNKDVKGA |
| NOV43f | CGSHLIVVVLFYGSGIFTYMRPNSKTTKELDKMISVFYTAVTPMLNPIIYSLRNKDVKGA |
| NOV43g | CGSHLIVVVLFYGSGIFTYMRPNSKTTKELDKMISVFYTAVTPMLNPIIYSLRNKDVKGA |
| NOV43a | LRKLVGRKCFSHRQ |
| NOV43b | LRKLVGRKCFSHRQ |
| NOV43c | LRKLVGRKCFSHRQ |
| NOV43d | LRKLVGRKCFSHRQ |
| NOV43e | LRKLVGRKCFSHRQ |
| NOV43f | LRKLVGRKCFSHRQ |
| NOV43g | LRKLVGRKCFSHRQ |
| NOV43a | (SEQ ID NO: 618) |
| NOV43b | (SEQ ID NO: 620) |
| NOV43c | (SEQ ID NO: 622) |
| NOV43d | (SEQ ID NO: 624) |
| NOV43e | (SEQ ID NO: 626) |
| NOV43f | (SEQ ID NO: 628) |
| NOV43g | (SEQ ID NO: 630) |

Further analysis of the NOV43a protein yielded the following properties shown in Table 43C.

TABLE 43C
Protein Sequence Properties NOV43a

| | |
|---|---|
| SignalP analysis: | Cleavage site between residues 42 and 43 |
| PSORT II analysis: | |

| PSG: | a new signal peptide prediction method | | | |
|---|---|---|---|---|
| | N-region: | length 11; | pos.chg 1; | neg.chg 2 |
| | H-region: | length 8; | peak value | 0.00 |

| | PSG score: −4.40 |
|---|---|
| GvH: | von Heijne's method for signal seq. recognition |
| | GvH score (threshold: −2.1): 0.24 |
| | possible cleavage site: between 41 and 42 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood =−13.32 | Transmembrane 25-41 |
| | INTEGRAL | Likelihood = −1.75 | Transmembrane 71-87 |
| | INTEGRAL | Likelihood = −8.86 | Transmembrane 101-117 |
| | INTEGRAL | Likelihood = −9.34 | Transmembrane 199-215 |
| | INTEGRAL | Likelihood = −1.65 | Transmembrane 241-257 |

| | |
|---|---|
| PERIPHERAL | Likelihood = 1.59 (at 273) |

| | ALOM score: −13.32 (number of TMSs: 5) |
|---|---|
| MTOP: | Prediction of membrane topology (Hartmann et al.) |
| | Center position for calculation: 32 |
| | Charge difference: 1.5 C(1.5) − N(0.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 8.77 |
| Hyd Moment(95): | 7.26 | G content: | 1 |
| D/E content: | 2 | S/T content: | 0 |
| Score: −7.02 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 8.0%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LFLIIYLLTVLGNQLIIILIFL at 30

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG55604-04 is end (k = 9)

A search of the NOV43a protein against the Geneseq database, a proprietary database that contains sequences published in patents and patent publication, yielded several homologous proteins shown in Table 43D.

TABLE 43D
Geneseq Results for NOV43a

| | | NOV43a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66338 | Human NOV 18 protein | 1 . . . 314 | 314/314 (100%) | 0.0 |
| | sequence - Homo sapiens, 314 aa. | 1 . . . 314 | 314/314 (100%) | |
| | [WO200155179-A2, 02- | | | |
| | AUG-2001] | | | |
| AAG66331 | Human NOV 11 protein | 1 . . . 314 | 314/314 (100%) | 0.0 |
| | sequence - Homo sapiens, 314 | 1 . . . 314 | 314/314 (100%) | |
| | aa. [WO200155179-A2, 02- | | | |
| | AUG-2001] | | | |
| AAG66337 | Human NOV 17 protein | 1 . . . 314 | 313/314 (99%) | 0.0 |
| | sequence - Homo sapiens, 314 | 1 . . . 314 | 314/314 (99%) | |
| | aa. [WO200155179-A2, 02- | | | |
| | AUG-2001] | | | |
| AAG66333 | Human NOV 13 protein | 1 . . . 314 | 313/314 (99%) | 0.0 |
| | sequence - Homo sapiens, 314 | 1 . . . 314 | 314/314 (99%) | |
| | aa. [WO200155179-A2, 02- | | | |
| | AUG-2001] | | | |
| AAG66332 | Human NOV 12 protein | 1 . . . 314 | 313/314 (99%) | 0.0 |
| | sequence - Homo sapiens, 314 | 1 . . . 314 | 314/314 (99%) | |
| | aa. [WO200155179-A2, 02- | | | |
| | AUG-2001] | | | |

In a BLAST search of public sequence databases, the NOV43a protein was found to have homology to the proteins shown in the BLASTP data in Table 43E.

TABLE 43E
Public BLASTP Results for NOV43a

| | | NOV43a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC69632 | Sequence 21 from Patent | 1 . . . 314 | 314/314 (100%) | e−180 |
| | WO0155179 - Homo sapiens | 1 . . . 314 | 314/314 (100%) | |
| | (Human), 314 aa. | | | |
| CAC69634 | Sequence 25 from Patent | 1 . . . 314 | 313/314 (99%) | e−180 |
| | WO0155179 - Homo sapiens | 1 . . . 314 | 314/314 (99%) | |
| | (Human), 314 aa. | | | |
| CAC69637 | Sequence 31 from Patent | 1 . . . 314 | 313/314 (99%) | e−180 |
| | WO0155179 - Homo sapiens | 1 . . . 314 | 314/314 (99%) | |
| | (Human), 314 aa. | | | |
| Q8NGH3 | Seven transmembrane helix | 1 . . . 314 | 312/314 (99%) | e−180 |
| | receptor - Homo sapiens | 1 . . . 314 | 314/314 (99%) | |
| | (Human), 314 aa. | | | |
| Q9EP67 | B5 olfactory receptor - | 1 . . . 304 | 253/304 (83%) | e−144 |
| | Mus musculus (Mouse), 307 aa. | 1 . . . 304 | 275/304 (90%) | |

PFam analysis indicates that the NOV43a protein contains the domains shown in the Table 43F.

TABLE 43F
Domain Analysis of NOV43a

| Pfam | NOV43a | Identities/Similarities | Expect |
|---|---|---|---|
| Domain | Match Region | for the Matched Region | Value |
| 7tm_1 | 41 . . . 290 | 58/277 (21%) | 8.4e−36 |
| | | 179/277 (65%) | |

### Example 44

The NOV44 clone was analyzed, and the nucleotide and encoded polypeptide sequences are shown in Table 44A.

TABLE 44A

NOV44 Sequence Analysis

| | | |
|---|---|---|
| NOV44a, CG55752-07 | SEQ ID NO: 659 | 1998 bp |
| DNA Sequence | ORF Start: ATG at 1 | ORF Stop: TGA at 1969 |

ATGGGCATTTATGGTTCAGTACCTTATCTCCTGGCCCACAAACTGGGCAGAACTATAGGTATTTTCTG
GCTGAATGCCTCGGAAACACTGGTGGAGATCAATACAGAGCCTGCAGTAGAGTACACACTGACCCAGA
TGGGCCCAGTTGCTGCTAAACAAAAGGTCAGATCTCGCACTCATGTGCACTGGATGTCAGAGAGTGGC
ATCATTGATGTTTTTCTGCTGACAGGACCTACACCTTCTGATGTCTTCAAACAGTACTCACACCTTAC
AGGTACACAAGCCATGCCCCCTCTTTTCTCTTTGGGATACCACCAGTGCCGCTGGAACTATGAAGATG
AGCAGGATGTAAAAGCAGTGGATGCAGGGTTTGATGAGCATGACATTCCTTATGATGCCATGTGGCTG
GACATAGAGCACACTGAGGGCAAGAGGTACTTCACCTGGGACAAAAACAGATTCCCAAACCCCAAGAG
GATGCAAGAGCTGCTCAGGAGCAAAAAGCGTAAGCTTGTGGTCATCAGTGATCCCCACATCAAGATTG
ATCCTGACTACTCAGTATATGTGAAGGCCAAAGATCAGGGCTTCTTTGTGAAGAATCGAGGAAGGGAA
GACTTTGAAGGGGTGTGTTGGCCAGGTCTCTCCTCTTACCTGGATTTCACCAATCCCAAGGTCAGAGA
GTGGTATTCAAGTCTTTTTGCTTTCCCTGTTTATCAGGGATCTACGGACATCCTCTTCCTTTGGAATG
ACATGAATGAGCCTTCTGTCTTTAGAGGGCCAGAGCAAACCATGCAGAAGAATGCCATTCATCATGGC
AATTGGGAGCACAGAGAGCTCCACAACATCTACGGTTTTTATCATCAAATGGCTACTGCAGAAGGACT
GATAAAACGATCTAAAGGGAAGGAGAGACCCTTTGTTCTTACACGTTCTTTCTTTGCTGGATCACAAA
AGTATGGTGGTGCCGTGTGGACAGGCGACAACACAGCAGAATGGAGCAACTTGAAAATTTCTATCCCA
ATGTTACTCACTCTCAGCATTACTGGGATCTCTTTTTGCGGAGCTGACATAGGCGGGTTCATTGGGAA
TCCAGAGACAGAGCTGCTAGTGCGTTGGTACCAGGCTGGAGCCTACCAGCCCTTCTTCCGTGGCCATG
CCACCATGAACACCAAGCGACGAGAGCCCTGGCTCTTTGGGGAGGAACACACCCGACTCATCCGAGAA
GCCATCAGAGAGCGCTATGGCCTCCTGCCATATTGGTATTCTCTGTTCTACCATGCACACGTGGCTTC
CCAACCTGTCATGAGGCCTCTGTGGGTAGAGTTCCCTGATGAACTAAAGACTTTTGATATGGAAGATG
AATACATGCTGGGTAGTGCATTATTGGTTCATCCAGTCACAGAACCAAAAGCCACCACAGTTGATGTG
TTTCTTCCAGGATCAAATGAGGTATGGTATGACTATAAGACATTTGCTCATTGGGAAGGAGGGTGTAC
TGTAAGATCCCAGTAGCCTTGGACACTATTCCAGTGTTTCAGCGAGGTGGAAGTGTGATACCCAATAA
AGACAACTGTAGGAAAATCCACAGGCTGGATGACTGAATCCTCCTATGGACTCCGGGTTGCTCTAAGC
ACTAAGGGTTCTTCATGGGTGAGTTATATCTTGATGATGGCCATTCATTCCAATACCTCACACCAGAA
GCAATTTTTGCACAGGAAGTTTTCATTCTGTTCCAGTGTTCTGATCAATTCCAGTTAAGCTGACCAGA
GGGGTCATTATCCCAGCAAGTGTGTGGTGGAGAAGATCTTGGTCTTAGGCTTCAGGAAGGAGCCATCT
TCTGTGACTACCCACTCATCTGGTGATGGTAAAGATCAGCCTGTGGCTTTTACGTATTGTGCCAAAAC
ATCCATCCTGAGCCTGGAGAAGCTCTCACTCAACATTGCCACTGACTGGGAGGTCCGCATAATATGAC
AAAGAACTGCCCCTGGTGATGTGAGC

| | | | |
|---|---|---|---|
| NOV44a, GG55752-07 | | | |
| Protein Sequence | SEQ ID NO: 660 | 656 aa | MW at 74971.6kD |

MGIYGSVPYLLAHKLGRTIGIFWLNASETLVEINTEPAVEYTLTQMGPVAAKQKVRSRTHVHWMSESG
IIDVFLLTGPTPSDVFKQYSHLTGTQANPPLFSLGYHQCRWNYEDEQDVKAVDAGFDEHDIPYDAMWL
DIEHTEGKRYFTWDKMRFPNPKRNQELLRSKXRKLVVISDPHIKIDPDYSVYVKAKDQGFFVKNQEGE
DFEGVCWPGLSSYLDFTNPKVREWYSSLFAFPVYQGSTDILFLWNDMNEPSVFRGPEQTMQKNAIHHG
NWEHRELHNIYGFYHQMATAEGLIKRSKGKERPFVLTRSFFAGSQKYGGAVWTGDNTAEWSNLKISIP
MLLTLSITGISFCGADIGGFIGNPETELLVRWYQAGAYQPFFRGHATMNTKRREPWLFGEEHTRLIRE
AIRERYGLLPYWYSLFYHAHVASQPVMRPLWVEFPDELKTFDMEDEYMLGSALLVHPVTEPKATTVDV
FLPGSNEVWYDYKTFAHWEGGCTVKIPVALDTIPVFQRGGSVIPIKTTVGKSTGWMTESSYGLRVALS
TKGSSVGELYLDDGHSFQYLHQKQFLHRKFSFCSSVLINSSFADQRGHYPSKCVVEKILVLGFRKEPS
SVTTHSSGDGKDQPVAFTYCAKTSILSLEKLSLNIATDWEVRII

| | | |
|---|---|---|
| NOV44b, CG55752-06 | SEQ ID NO: 661 | 2001 bp |
| DNA Sequence | ORF Start: ATG at 1 | ORF Stop: TGA at 1972 |

ATGGGCATTTATGGTTCAGTACCTTATCTCCTGGCCCACAAACTGGGCAGAACTATAGGTATTTTCTG
GCTGAATGCCTCGGAAACACTGGTGGAGATCAATACAGAGCCTGCAGTAGAGTACACACTGACCCAGA
TGGGCCCAGTTGCTGCTAAACAAAAGGTCAGATCTCGCACTCATGTGCACTGGATGTCAGAGAGTGGC
ATCATTGATGTTTTTCTGCTGACAGGACCTACACCTTCTGATGTCTTCAAACAGTACTCACACCTTAC
AGGTACACAAGCCATGCCCCCTCTTTTCTCTTTGGGATACCACCAGTGCCGCTGGAACTATGAAGATG
AGCAGGATGTAAAAGCAGTGGATGCAGGGTTTGATGAGCATGACATTCCTTATGATGCCATGTGGCTG
GACATAGAGCACACTGAGGGCAAGAGGTACTTCACCTGGGACAAAAACAGATTCCCAAACCCCAAGAG
GATGCAAGAGCTGCTCAGGAGCAAAAAGCGTAAGGTACTTGTGGTCATCAGTGATCCCCACATCAAGA
TTGATCCTGACTACTCAGTATATGTGAAGGCCAAAGATCAGGGCTTCTTTGTGAAGAATCAGGAAGGG
GAAGACTTTGAAGGGGTGTGTTGGCCAGGTTCTCCTCTTACCTGGATTTCACCAATCCCAAGGTACAG
AGAGTGGTATTCAAGTCTTTTTGCTTTCCCTGTTTATCAGGGATCTACGGACATCCTCTTCCTTTGGA
ATGACATGAATGAGCCTTCTGTCTTTAGAGGGCCAGAGCAAACCATGCAGAAGAATGCCATTCATCAT
GGCAATTGGGAGCACAGAGAGCTCCACAACATCTACGGTTTTTATCATCAAATGGCTACTGCAGAAGG
ACTGATAAAACGATCTAAAGGGAAGGAGAGACCCTTTGTTCTTACACGTTCTTTCTTTGCTGGATCAC
AAAAGTATGGTGCCGTGTGGACAGGCGACAACACAGCAGAATGGAGCAACTTGAAAATTTCTATCCCA
ATGTTACTCACTCTCAGCATTACTGGGATCTCTTTTTGCGGAGCTGACATAGGCGGGTTCATTGGGAA
TCCAGAGACAGAGCTGCTAGTGCGTTGGTACCAGGCTGGAGCCTACCAGCCCTTCTTCCGTGGCCATG
CCACCATGAACACCAAGCGACGAGAGCCCTGGCTCTTTGGGGAGGAACACACCCGACTCATCCGAGAA
GCCATCAGAGAGCGCTATGGCCTCCTGCCATATTGGATTCTCTGTTCTACCATGCACACGTGGCATTC
CCAACCTGTCATGAGGAGGCCTCTGTGGGTAGAGTTCCCTGATGAACTAAAGACTTTTGATATGGAAG
ATGAATACATGCTGGGTTTAGGGAGTGCATTATTGGTTCATCCAGTCACAGAACCAAAAGCCACCACA
GTTGATGTGTTTCTTTCCAGGATCAAATGAGGTATGGTATGACTATAAGACATTGCTCATTGGGAAGG
AGGGTGTACTGTAAAGATCCCAGTAGCCTTGGACACTATTCCAGTGTTTCAGCGAGGTGGAAGTGTGA
TACCAATAAAGACAACTGTAGGAAAATCCACAGGCTGGATGACTGAATCCTCCTATGGACTCCGGGTT
GCTCTAAGCACTGGTTCTTCAGTGGGTGAGTTATATCTTGATGATGGCCATTCATTCCAATACCTCCA
CCAGAAGCAATTTTTGCACAGGAAGTTTTCATTCTGTTCCAGTGTTCTGATCAATTCCAGTTTTGCTG
ACCAGAGGGGTCATTATCCCAGCAAGTGTGTGGTGGAGAAGATCTTGGTCTTAGGCTTCAGGAAGGAG
CCATCTTCTGTGACTACCCACTCATCTGATGGTAAAGATCAGCCTGTGGCTTTTACGTATTGTGCCAA
AACATCCATCCTGAGCCTGGAGAAGCTCTCACTCAACATTGCCACTGACTGGGAGGTCCGCATCATAT
GACAAAGAACTGCCCCTGGTGATGTGAGC

| | | | |
|---|---|---|---|
| NOV44b, CG55752-06 | | | |
| Protein Sequence | SEQ ID NO: 662 | 657 aa | MW at 75166.9kD |

MGIYGSVPYLLAHKLGRTIGIFWLNASETLVEINTEPAVETLTQMGPVAAKQKVRSRTHVHWAMSESG
IIDVFLLTGPTPSDVFKQYSHLTGIQAMPPLFSLGYHQCRWNYEDEQDVKAVDAGFDEHDIPYDAMWL
DIEHTEGKRYFTWDKNRFPNPKRMQELLRSKKRKVLVVISDPHIKIDPDYSVYKVAKDQGFFVKNQEG
EDFEGVCWPGLSSYLDFTNPKVREWYSSLFAFPVYQGSTDILFLWNDMNEPSVRGPEQTMQKNAAIHH
GNWEHRELHNIYGFYHQMATAEGLIKRSKGKERPFVLTRSFFAGSQKYGAVWTGDNTAEWSNLKISIP
MLLTLSITGISFCGADIGGFIGNPETELLVRWYQAGAYQPFFRGHATMNTKRREPWLFGEEHTRLIRE
AIRERYGLLPYWYSLFYHAHVASQPVMRRPLWVEFPDELKTFDMEDEYMLGLGSALLVHPVTEPKATT
VDVFLPGSNEVWYDYKTFAHWEGGCTVKIPVALDTIPVFQRGGSVIPIKTTVGKSTGWMTESSYGLRV
ALSTGSSVGELYDDGHSFQYLHQKQFLHRKFSFCSSVLINSSFADQRGHYPSKCVVEAKILVLGFRKE
PSSVTTHSSDGKDQPVAFTYCAKTSILSLEKSLNIATDWEVRII

| | | |
|---|---|---|
| NOV44c, CG55752-01 | SEQ ID NO: 663 | 3025 bp |
| DNA Sequence | ORF Start: ATG at 28 | ORF Stop: TGA at 2929 |

ACAGTGCCTGGGGGTCAGGCTTCCGCATGCGGGCTGCAGTTGCTGGCATTGCCTTCCGCAGGGAGGCG
TCAGAAACAGTGGCTTTCCAAGAAGTCCACCTATCAGGCATTATTGGATTCAGTCACAACAGATAAAG
ACAGCACCAGGTTCCAAATCATCAATGAAGCAAGTAAGGTGAGGCGTCAGAAACAGTGGCTTTCCAAG
AAGTCCACCTATCAGGCATTATTGGATTCAGTCACAACAGATGAAGACAGCACCAGGTTCCAAATCAT
CAATGAAGCAAGTAAGGTGCCTCTCCTGGCTGAAATTTATGGTATAGAAGGAAACATTTTCAGGCTTA
AAATTAATGAAGAGACTCCTCTAAAACCCAGATTTGAAGTTCCGGATGTCCTCACAAGCAAGCCAAGC
ACTGTAAGGATTTCATGCTCTGGGGACACAGGCAGTCTGATATTGGCAGATGGAAAAGGAGACCTGAA
GTGCCATATCACAGCAAACCCATTCAAGGTAGACTTGGTGTCTGAAGAAGAGGTTGTGATTAGCATAA
ATTCCCTGGGCCAATTATACTTTGAGCATGGCAGGGCCCCTAGGGTCTCTTTCTCGGATAAGGTTAAT
CTCACGCTTGGTAGCATATGGGATAAGATCAAGAACCTTTTCTCTAGGCAAGGATCAAAAGACCCAGC
TGAGGGCGATGGGGCCCAGCCTGAGGAAACACCCAGGGATGGCGACAAGCCAGAGGAGACTCAGGGGA
AGGCAGAGAAAGATGAGCCAGGAGCCTGGGAGGAGACATTCAAAACTCACTCTGACAGCAAGCCGTAT
GGCCCTTCTTCTATTGGTTTGGATTTCTCCTTGCATGGATTTGAGCATCTTTATGGGATCCCACAACA
TGCAGAATCACACCAACTTAAAAATACTGGGGATGGAGATGCTTACCGTCTTTATAACCTGGATGTCT
ATGGATACCAAATATATGATAAATGGGCATTTATGGTTCAGTACCTTATCTCCTGGCCCAACAAACTG
GGCAGAACTATAGGTATTTTCTGGCTGAATGCCTCGGAACACTGGTGGAGATCAATACAGAGCACTGC
AGGGATAGTCATCTTTGGTCCTGTCTCTTTGATTTATCAAAGCCAGGGAGATACACCTCTAACAACTC
ATGTGCACTGGATGTCAGAGAGTGGCATCATTGATGTTTTTCTGCTGACAGGACCTACACCTTCTGAT
GTCTTCAAACAGTACTCACACCTTACAGGTACACAAGCCATGCCCCCTCTTTTCTCTTTGGGATACCA
CCAGTGCCGTCGGAACTATGAAGATGAGCAGGATGTAAAAGCAGTGGATGCAGGGTTTGATGAGCATG
ACATTCCTTATGATGCCATGTGGCTGGACATAGAGCACACTGAGGGCAAGAGGTACTTCACCTGGGAC
AAAAACAGATTCCCAAACCCCAAGAGGATGCAAGAGCTGCTCAGGAGCAAAAAGCGTAAGCTTGTGGT
CATCAGTGATCCCCACATCAAGATTGAACCTGACTACTCAGTATATGTGAAGGCCAAAGATCAGGGCT
TCTTTGTGAAGAATCAGGAAGGGGAAGACTTTGAAGGGGTGTGTTGGCCAGGTATGAAATCATACCTG
GATTCACCAATCCCAAGGTCAGAGAGTGGTATTCAAGTATGTTCAGTTCCAATTGTGAATGGATCTAC
GGACATCCTCTTCCTTTGGAATGACATGAATGAGCCTTCTGTCTTTAGAGGGCCAGAGCAAACCATGC
AGAAGAATGCCATTCATCATGGCAATTGGGAGCAGAGAGAGCTCCACAACATCTACGGTTTTTATATG
GCTACTGCAGAAGGACTGTAAAACGATCTAAAGGGAAGGAGAGACCCTTTGTTCTTAACACGTTCTTT
CTTTGCTGGATCACAAAAGTATGGTGCCGTGTGGACAGGCGACAACACAGCAGAATGGAGCAACTTGA
AAATTTCTATCCCAATGTTACTCACTCTCAGCATTACTGGGATCTCTTTTTGCGGAGCTGACATAGGC
GGGTTCATTGGGAATCCAGAGAGCAGAGCTGCTAGTGCGGTGGTACCAGGCTGGGACCTACGCACCTT
CTTCCGTGGCCATGCCACCATGAACACCAAGCGCGAGAGCCCTGGCTCTTTGGGGAGGAACACAACCC
GACTCATCCGAGAAGCCATCAGAGAGCGCTATGGCCTCCTGCCATATTGGTATTCTCTCTTCTACCAT
GCACACGTGGCTTCCCAACCTGTCATGAGGCCTCTGTGGGTAGAGTTCCCTGATGAACTAAAGACTTT
TGATATGGAAGATGAATACATGTTAGGGAGTGCATTATTGGTTCATCCAGTCACAGAACCAAAAGCCA
CCACAGTTGATGTGTTTCTTCCAGGATCAAATGAGGTAGTCTGGTATGACTATAAGACATTTGCTCAT
TGGGAAGGAGGGTGTACTGTAAAGATCCCAGTACTGTTACAGATTCCAGTGTTTCAGCGAGGTGGAAG
GGGTTGCTCTAAGCACTCTCCAGGGTTCTTCAGTGGGTGAGTTATATCTTGATGATGGCCATTCATTC
CAATACCTCCACCAGAAGCAATTTTTGCACAGGAAGTTTTCATTCTGTTCCAGTGTTCTGGTGGCCTC
CTCTCCAGTATCTCAAGGACACTTACATACCCCACTCAGCATGACAAAAGCCCTGCTTTTCACTGTAT
CGTCTCCAGCCAGCGTGAAAATGCGGCTTCACTACAGCCCAGAGAAAAGGGCCAGGTTTAGTCATTGT
GCCAAAACATCCATCCTGAGCCTGGAGAAGCTCTCACTCAACATTGCCACTGACTGGGAGGTCCGCAT
CATATGACAAAGAACTGCCCCTGGTGATGTGAGCAGGGACCTGCCTGCCCCTTTCAACCTTTCCCCTC
ACCTTTTTTGAGATTTTTGCTGCAATCTGTTTG

| | | | |
|---|---|---|---|
| NOV44c, CG55752-01 | | | |
| Protein Sequence | SEQ ID NO: 664 | 967 aa | MW at 109801.3kD |

MRAAVAGIAFRRRRQKQWLSKKSTYQALLDSVTTDEDSTRFQIINEASKVRRQKQWLSKKSTYQALLD
SVTTDEDSTRFQIINEASKVPLLAEIYGIEGNIFRLKINEETPLKPRFEVPDVLTSKPSTVRISCSGD
TGSLILADGKGDLKCHITANPFKVDLVSEEEVVISINSLGQLYFEHGRAPRVSFSDKVNLTLGSIWDK
IKNLFSRQGSKDPAEGDGAQPEETPRDDKPEETQGKAEKDEPGAWEETFKTHSDSKPYGPSSAIGLDF
SLHGFEHLYGIPQHAESHQLKNTGDGDAYRLYNLDVYGYQIYDKMGIYGSVPYLLAHKLGRTIGIFWL
NASETLVEINTEPAGIVIFGPVSLIYQSQGDTPLTTHVHWMSESGIIDVFLLTGPTPSDVFKQYSHLT
GTQAMPPLFSLGYHQCRWNYEDEQDVKAVDAGFDEHDIPYDAMWLDIEHTEGKRYFTWDKNRFPNPKR
MQELLRSKKRKLVVISDPHIKIEPDYSVYVKAKDQGFFVKNQEGEDFEGDVCWPGMKSYLFTNPKVRE
WYSSMFSSNCDGSTDILFLWNDMNEPSVFRGPEQTMQKNAIHHGNWEHRELHNIYGFYMATEAGLIKR
SKGKERPFVLTRSFFAGSQKYGAVWTGDNTAEWSNLKISIPMLLTLSITGISFCGADIGGFIGNPETE
LLVRWYQAGAYQPFFRGHATMNTKRREPWLFGEEHTRLIREAIRERYGLLPYWYSLFYHAHVASQPVM
RPLWVEFPDELKTFDMEDEYMLGSALLVHPVTEPKATTVDVFLPGSNEVVWYDYKTFAHWEGGCTVKI
PVLLQIPVFQRGGSVIPIKTTVGKSTGWMTESSYGLRVALSTLQGSSVGELYLDDGHSFQYLHQKQFL
HRKFSFCSSVLVASSPVSQGHLHTPLSMTKALLFTVSSPASVKMRLHYSPEKRARFSHCAATSISSLE
KLSNIATDWEVRII

| | | |
|---|---|---|
| NOV44d, CG55752-02 | SEQ ID NO: 665 | 4483 bp |
| DNA Sequence | ORF Start: ATG at 204 | ORF Stop: TGA at 2946 |

AACGCTAGTTTGGGCCTGAAAAATTCCAGGAGCAAGAGTCAAGATTTGTCACTCCATGAGAATCTGGA
GGGGACTCCCTTCCCAGAAACTTGACGATGAAGTACTGGTTGTAATTTTAGAAAGACACCCAATCGGC
TTTTTTAAAAGATCGCCCAGGGCCCTTGTCCTGAGAGCTGGGAGCTGGTCGGAGTGACAGAGAAGCCA
TGGAAGCAGCAGTGAAAGAGGAAATAAGTGTTGAAGATGAAGCTGTAGATAAAAACATTTTCAGAGAC
TGTAACAAGATCGCATTTTACAGGCGTCAGAAACAGTGGCTTTCCAAGAAGTCCACCTATCAGGCATT
ATTGGATTCAGTCACAACAGATGAAGACAGCACCAGGTTCCAAATCATCAATGAAGCAAGTAAGGTTC
CTCTCCTGGCTGAAATTTATGGTATAGAAGGAAACATTTTCAGGCTTAAAATTAACGAAGAGACTCCT
CTAAAACCCAGATTTGAAGTTCCGGATGTCCTCACAAGCAAGCCAAGCACTGTAAGGCTGATTTCATG
CTCTGGGGACACAGGCAGTCTGATATTGGCAGATGGAAAAGGAGACCTGAAGTGCCATATCACAGCAA
ACCCATTCAAGGTAGACTTGGTGTCTGAAGAAGAGGTTGTGATTAGCATAAATTCCCTGGGCCAATTA
TACTTTGAGCATCTACAGATTCTTCACAAACAAAGAGCTGCTAAAGAAAATGAGGAGGAGACATCAGT
GGACACCTCTCAGGAAAATCAAGAAGATCTGGGCCTGTGGGAAGAGAAATTTGGAAAATTTGTGGATA
TCAAAGCTAATGGCCCTCTTCTATTGGTTTGGATTTCTCCTTAGCATGGATTTGAGCATCTTTATGGG
ATCCCACAACATGCAGAATCACACCAACTTAAAAATACTGGTGATGGAGATGCTTACCGTCTTTATAA
CCTGGATGTCTATGGATACCAAATATATGATAAAATGGGCATTTATGTTCAGTACCTTATCTCCTAGG
CCCACAAACTGGGCAGAACTATAGGTATTTTCTGGCTGAATGCCTCGGAAACACTGGTGGAGATCAAT
ACAGAGCCTGCAGTAGAGTACACACTGACCCAGATGGGCCCAGTTGCTGCTAAACAAAAGGTCAGATC
TCGCACTCATGTGCACTGGATGTCAGAGAGTGGCATCATTGATGTTTTTCTGCTGACATTACCTACAC
CTTCTGATGCTTCAAACAGTACTCACACCTTACAGGCACACAAGCCATGCCCCCTCTTTTTCTCTTTG
GGATACCACCAGTGCCGCTGGAACTATGAAGATGAGCAGGATGTAAAAGCAGTGGATGCAGGGTTTGA
TGAGCATGACATTCCTTATGATGCCATGTGGCTGGACATAGAGCACACTGAGGGCAAGAGGTACTTCA
CCTGGGACAAAAACAGTTCCCAAACCCCAAGAGGATGCAAGAGCTGCTCAGGAGCAAAAAAGCGTAAG
CTTGTGGTCATCAGTGAATCCCCACATCAAGATTGATCCTGACTACTCAGTATATGTGAAGGCCAAAG
CAGGGATCTACGGACATCCTCTTCCTTTGGAATGACATGAATGAGCCTTCTGTCTTTAGAGGGCCAGA
GCAAACCATGCAGAAGAATGCCATTCATCATGGCAATTGGGAGCACAGAGAGCTCCACAACATCTACG
GTTTTTATCATCAAATGGCTACTGCAGAAGGACTGATAAAACGATCTAAAGGGAAGGAGAGACCCTTT
GTTCTTACACGTTCTTTCTTTGCTGGATCACAAAAGTATGGTGCCGTGTGGACAGGCGACAACACAGC
AGAATGGAGCAACTTGAAAATTTCTATCCCAATGTTACTCACTCTCAGCATTACTGGGATCTCTTTTT
GCGGAGCTGACATAGGCGGGTTCATTGGGAATCCAGAGACAGAGCTGCTAGTGCGTTGGTACCAGGCT
GGAGCCTACCAGCCCTTCTTCCGTGGCCATGCACCATGAACACCAAGCGACGAGAGCCCTGAGCTCTT
TGGGGAGGAACACACCCGACTCATCCGAGAACCATCAGAGAGCGCTATGGCCTCCTGCCATAATTGGT
ATTCTCTGTTCTACCATGCACACGTGGCTTCCCAACCTGTCATGAGGCCTCTGTGGGTAGAGTTCCCT
GATGAACTAAAGACTTTTGATATGGAAGATGAATACATGCTGGGGAGTGCATTATTGGTTCATCCAGT
CACAGAACCAAAAGCCACCACAGTTGATGTGTTTCTTCCAGGATCAAATGAGGTCTGTATGCACTATA
AGACATTTGCTCATTGGGAAGGAGGGTGTACTGTAAAGATCCCAGTAGCCTTGGACACTATTCCAGTG
TTTCAGCGAGGTGGAAGTGTGATACCAATAAAGACAACTGTAGGAAAATCCACAGGCTGGATGACTGA
ATCCTCCTATGGACTCCGGGTTGCTCTAAGCACTAAGGGTTCTTCAGTGGGTGAGTTATATCTTGATG
ATGGCCATTCATTCCAATACCTCCACCAGAAGCAATTTTTGCACAGGAAGTTTTCATTCTGTTCCAGT
GTTCTGATCAATAGTTTTGCTGACCAGAGGGGTCATTATCCCAGCAAGTGTGTGGTGGAGAAGATCTT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GACTGGGAGGTCCGCATCATATGACAAAGAACTGCCCCTGGTGATGTGAGCAGGGACCTGCCTGCCCC
TTTCAACCTTTCCCCTCACCTTTTTTGAGATTTTTGCTGCAATCTGTTTGCCTTCCCTGAATCAAAAT
AATCTTTCATTCGTCACCATTATACTAATGAACAATAGATTATCATGTTCAAAATTTCAGATTTTACA
TGTTAAGATGTACTAACAATATTCCTTGTATCAAACATCTCCTTTTCTCCCTGATACATAGCCCTGAG
ACATTTATAGCGTTCAGGAGTCTTCTATTGCTTCCATTCCTTCAGCAGGGCTGCGTGGGTCTGTTTTA
ACGTGGGCCAAGCCTACCTGGGCAGCCCATTTGCCAGGGCTTGCCTCAGGCCATGCAGCATTGGCGCT
CTGGCTGCAGCAGCTGAGTTGCTCAAGGCCAGTGTCCAAGTGGACAGCAGCCTCTGGTACTCCCCCCA
GTTATCTTCCACCCACATGGACTGGGCAGAGCAGCCCTCTTCTGTGTGCACTGCATACGTGCAGCCCG
TGGGAGTTATTCTCCCCTAGAGATCGACTTGGCAGCACGAAGGATTCTTTTCTCTTTCATGCTTCTCA
GGCTCAATAGTTTCTAATTAATCTTAAAATCCATGTCTTTTACATTGTTTTTTTAATTAAGTGCTGTT
TACTAACCAATAATATTTATAACATGAGTAAGCTATAATTAATAACAATGAAATAAAAATACCATGTA
CCCACCACTGGACTTCAGAAGTAGAACTCATGACTGGGCTAGGATGAGGCAAGGGAGAACCCTGGCCT
TGGGCACAAAATGTAAGGGATGCCAAAAAAATACAGTAATCAAAGTAAGTAATATTTCAATCCAATAT
GGATTAGTATTACTGAGTTTCCTTTTGTCCCAGGCTTTAAATATGGCTTGGCATGGGGCAGAACATTA
CAACATACCAGTCGTGTCATGGTGCCCAAGGCTCCACAGACCTCAGTGGCTCCCTGCTGCCTGCCACA
GCATCTGTTTTAGCAGCCTCGACTCCTCAGCACTCCTCAGCACACACCTCTTCTTATCAGGCTTCCTC
CACTTAGCAACTTGCTAACGGCCACCTCTGTGCCTTCTGATCCCTGGGCGCCAATATCCTCCTGCCCT
TACCATCCTTCCAGGCCCAACTTAAATCCCATTTCCCATGAAGCCTAAGCTGAACACCCCTACCAGAT
CCCATACCATTAGCAGTGATTTTGCCTTCCCCGTAATGCTGTCCCACTTATAACTGTAAGCTCTACTT
GCTGTGATTAGTTAGCTGGTGGATTTAATTGATTAAAAAAATTACGATTGAATGTAAAAAAAAA

| | | | |
|---|---|---|---|
| NOV44d, CG55752-02 | | | |
| Protein Sequence | SEQ ID NO: 666 | 914 aa | MW at 104319.2kD |

MEAAVKEEISVEDEAVDKNIFRDCNKIAFYRRQKQWLSKKSTYQALLDSVTTDEDSTRFQIINEASKV
PLLAEIYGIEGNIFRLKINEETPLKPRFEVPDVLTSKPSTVRLISCDGDTGSLILADGKGDLKCHITA
NPFKVDLVSEEEVVISINSLGQLYFEHLQILHKQRAAKENEEETSVDTSQENQEDLGLWEEKFGKFVD
IKANGPSSIGLDFSLHGFEHLYGIPQHAESHQLKNTGDGDAYRLYNLDVYGYQIYDKMGIYGSVPYLL
AHKLGRTIGIFWLNASETLVEINTEPAVEYTLTQMGPVAAKQKVRSRTHVHWMSESGIIDVFLLTGPT
PSDVFKQYSHLTGTQAMPPLFSLGYHQCRWNYEDEQDVKAVDAGFDEHDIPYDAMWLDIEHEEGKRYF
TWDKNRFPNPKRIVIQELLRSKKRKLWISDPHIKIDPDYSVVKAKDQGFFVKNQEGEDFEGVCWPGLS
SYLDFTNPKVREWYSSLFAFPVYQGSTDILFLWNDMNEPSVFRGPEQTMQKNAIHHGNWEHRILHNIY
GFYHQMATAEGLIKRSKGKERPFVLTRSFFAGSQKYGAVWTGDNTAEWSNLKISIPMLLTLSITGISP
CGADIGGFIGNPETELLVRWYQAGAYQPFFRGHATMNTKRREPWLFGEEHTRLIREAIRERYGLLPYW
YSLFYHAHVASQPVMRPLWVEFPDELKTFDMEDEYMLGSALLVHPVTEPKATTVDVFLPGSNEVWYDY
KTFAHWEGGCTVKIPVALDTIPVFQRGGSVIPIKTTVGKSTGWMTESSYGLRVALSTKGSSVGELYLD
DGHSFQYLHQKQFLHRKFSFCSSVLINSFADQRGHYPSKCVVEKILVLGFRKEPSSVTTHSSDGKDQP
VAFTYCAKTSILSLEKLSLNIATDWEVRII

| | | |
|---|---|---|
| NOV44e, CG55752-03 | SEQ ID NO: 667 | 3015 bp |
| DNA Sequence | ORF Start: ATG at 204 | ORF Stop: TGA at 2946 |

AACGCTAGTTTGGGCCTGAAAAATTCCAGGAGCAAGAGTCAAGATTTGTCACTCCATGAGAATCTGGA
GGGACTCCCTTCCCAGAAACTTGACGATGAAGTACTGGTTGTAATTTTAGAAAGACACCCAATCAGGC
TTTTTTAAAAGATCGCCCAGGGCCCTTGTCCTGAGAGCTGGGAGCTGGTCGGAGTGACAGAGAAGCCA
TGGAAGCAGCAGTGAAAGAGGAAATAAGTGTTGAAGATGAAGCTGTAGATAAAAACATTTTCAGAGAC
TGTAACAAGATCGCATTTTACAGGCGTCAGAAACAGTGGCTTTCCAAGAAGTCCACCTATCAGGCATT
ATTGGATTCAGTCACAACAGATGAAGACAGCACCAGGTTCCAAATCATCAATGAAGCAAGTAAGGTTC
CTCTCCTGGCTGAAATTTATGGTATAGAAGGAAACATTTTCAGGCTTAAAATTAACGAAGAGACTCCT
CTAAAACCCAGATTTGAAGTTCCGGATGTCCTCACAAGCAAGCCAAGCACTGTAAGGCTGATTTCATG
CTCTGGGGACACAGGCAGTCTGATATTGGCAGATGGAAAAGGAGACCTGAAGTGCCATATCACAGCAA
ACCCATTCAAGGTAGACTTGGTGTCTGAAGAAGAGGTTGTGATTAGCATAAATTCCCTGGGAAAATTA
GTTCTTACACGTTCTTTCTTTGCTGGATCACAAAAGTATGGTGCCGTGTGGACAGGCGACAACACAGC
AGAATGGAGCAACTTGAAAATTTCTATCCCAATGTTACTCACTCTCAGCATTACTGGGATCTCTTTTT
GCGGAGCTGACATAGGCGGGTTCATTGGGAATCCAGAGACAGAGCTGCTAGTGCGTTGGTACCAGGCT
GGAGCCTACCAGCCCTTCTTCCGTGGCCATGCACCATGAACACCAAGCGACGAGAGCCCTGAGCTCTT
TGGGGAGGAACACACCCGACTCATCCGAGAACCATCAGAGAGCGCTATGGCCTCCTGCCATAATTGGT
ATTCTCTGTTCTACCATGCACACGTGGCTTCCCAACCTGTCATGAGGCCTCTGTGGGTAGAGTTCCCT
GATGAACTAAAGACTTTTGATATGGAAGATGAATACATGCTGGGGAGTGCATTATTGGTTCATCCAGT
CACAGAACCAAAAGCCACCACAGTTGATGTGTTTCTTCCAGGATCAAATGAGGTCTGTATGCACTATA
AGACATTTGCTCATTGGGAAGGAGGGTGTACTGTAAAGATCCCAGTAGCCTTGGACACTATTCCAGTG
TTTCAGCGAGGTGGAAGTGTGATACCAATAAAGACAACTGTAGGAAAATCCACAGGCTGGATGACTGA
ATCCTCCTATGGACTCCGGGTTGCTCTAAGCACTAAGGGTTCTTCAGTGGGTGAGTTATATCTTGATG
ATGGCCATTCATTCCAATACCTCCACCAGAAGCAATTTTTGCACAGGAAGTTTTCATTCTGTTCCAGT
GTTCTGATCAATAGTTTTGCTGACCAGAGGGGTCATTATCCCAGCAAGTGTGTGGTGGAGAAGATCTT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GTTCTTACACGTTCTTTCTTTGCTGGATCACAAAAGTATGGTGCCGTGTGGACAGGCGACAACACAGC
AGAATGGAGCAACTTGAAAATTTCTATCCCAATGTTACTCACTCTCAGCATTACTGGGATCTCTTTTT
GCGGAGCTGACATAGGCGGGTTCATTGGGAATCCAGAGACAGAGCTGCTAGTGCGTTGGTACCAGGCT
GGAGCCTACCAGCCCTTCTTCCGTGGCCATGCACCATGAACACCAAGCGACGAGAGCCCTGAGCTCTT
TGGGGAGGAACACACCCGACTCATCCGAGAACCATCAGAGAGCGCTATGGCCTCCTGCCATAATTGGT
ATTCTCTGTTCTACCATGCACACGTGGCTTCCCAACCTGTCATGAGGCCTCTGTGGGTAGAGTTCCCT
GATGAACTAAAGACTTTTGATATGGAAGATGAATACATGCTGGGGAGTGCATTATTGGTTCATCCAGT
CACAGAACCAAAAGCCACCACAGTTGATGTGTTTCTTCCAGGATCAAATGAGGTCTGTATGCACTATA
AGACATTTGCTCATTGGGAAGGAGGGTGTACTGTAAAGATCCCAGTAGCCTTGGACACTATTCCAGTG
TTTCAGCGAGGTGGAAGTGTGATACCAATAAAGACAACTGTAGGAAAATCCACAGGCTGGATGACTGA
ATCCTCCTATGGACTCCGGGTTGCTCTAAGCACTAAGGGTTCTTCAGTGGGTGAGTTATATCTTGATG
ATGGCCATTCATTCCAATACCTCCACCAGAAGCAATTTTTGCACAGGAAGTTTTCATTCTGTTCCAGT
GTTCTGATCAATAGTTTTGCTGACCAGAGGGGTCATTATCCCAGCAAGTGTGTGGTGGAGAAGATCTT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GTTCTGATCAATAGTTTTGCTGACCAGAGGGGTCACTATCCAGCAAGTGTGGGTGGAGAAAGATCTAT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GACTGGGAGGTCCGCATCATATGACAAAGAACTGCCCCTGGTGATGTGAGAGGGACCATGCCTGCCCC
TTTCAACCTTTCCCCTCACCTTT

| | | | |
|---|---|---|---|
| NOV44e, CG55752-03 | | | |
| Protein Sequence | SEQ ID NO: 668 | 914 aa | MW at 104206.0kD |

MEAAVKEEISVEDEAVDKNIFRDCNKIAFYRRQKQWLSKKSTYQALLDSVTTDEDSTRFQIINEASKV
PLLAEIYGIEGNIFRLKINEETPLKPRFEVPDVLTSKPSTVRLISCDGDTGSLILADGKGDLKCHITA
NPFKVDLVSEEEVVISINSLGQLYFEHLQILHKQRAAKENEEETSVDTSQENQEDLGLWEEKFGKFVD
IKANGPSSIGLDFSLHGFEHLYGIPQHAESHQLKNTGDGDAYRLYNLDVYGYQIYDKMGIYGSVPYLL
AHKLGRTIGIFWLNASETLVEINTEPAVEYTLTQMGPVAAKQKVRSRTHVHWMSESGIIDVFLLTGPT
PSDVFKQYSHLTGTQAMPPLFSLGYHQCRWNYEDEQDVKAVDAGFDEHDIPYDAMWLDIEHEEGKRYF
TWDKNRFPNPKRIVIQELLRSKKRKLWISDPHIKIDPDYSVVKAKDQGFFVKNQEGEDFEGVCWPGLS
SYLDFTNPKVREWYSSLFAFPVYQGSTDILFLWNDMNEPSVFRGPEQTMQKNAIHHGNWEHRILHNIY
GFYHQMATAEGLIKRSKGKERPFVLTRSFFAGSQKYGAVWTGDNTAEWSNLKISIPMLLTLSITGISP
CGADIGGFIGNPETELLVRWYQAGAYQPFFRGHATMNTKRREPWLFGEEHTRLIREAIRERYGLLPYW
YSLFYHAHVASQPVMRPLWVEFPDELKTFDMEDEYMLGSALLVHPVTEPKATTVDVFLPGSNEVWYDY
KTFAHWEGGCTVKIPVALDTIPVFQRGGSVIPIKTTVGKSTGWMTESSYGLRVALSTKGSSVGELYLD
DGHSFQYLHQKQFLHRKFSFCSSVLINSFADQRGHYPSKCVVEKILVLGFRKEPSSVTTHSSDGKDQP
VAFTYCAKTSILSLEKLSLNIATDWEVRII

| | | |
|---|---|---|
| NOV44f, CG55752-04 | SEQ ID NO: 669 | 3102 bp |
| DNA Sequence | ORF Start: ATG at 103 | ORF Stop: TGA at 2839 |

TACTGGTTGTAATTTTAGAAAGACACCCAATCGGCTTTTTTAAAAGATCGCCCAGGGCCCTTGTCCTG
AGAGCTGGGAGCTGGTCGGAGTGACAGAGAAGCCATGGAAGCAGCAGTGAAAGAGGAAATAAGTGTTG
AAGATGAAGCTGTAGATAAAAACATTTTCAGAGACTGTAACAAGATCGCATTTTACAGGCGTCAGAAA
CAGTGGCTTTCCAAGAAGTCCACCTATCGGGCATTATTGGATTCAGTCACAACAGATGAAGACAGCAC
CAGGTTCCAAATCATCAATGAAGCAAGTAAGGTTCCTCTCCTGGCTGAAATTTATGGTATAGAAGGAA
AGACATTTGCTCATTGGGAAGGAGGGTGTACTGTAAAGATCCCAGTAGCCTTGGACACTATTCCAGTG
TTTCAGCGAGGTGGAAGTGTGATACCAATAAAGACAACTGTAGGAAAATCCACAGGCTGGATGACTGA
ATCCTCCTATGGACTCCGGGTTGCTCTAAGCACTAAGGGTTCTTCAGTGGGTGAGTTATATCTTGATG
ATGGCCATTCATTCCAATACCTCCACCAGAAGCAATTTTTGCACAGGAAGTTTTCATTCTGTTCCAGT
GTTCTGATCAATAGTTTTGCTGACCAGAGGGGTCATTATCCCAGCAAGTGTGTGGTGGAGAAGATCTT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GTTCTTACACGTTCTTTCTTTGCTGGATCACAAAAGTATGGTGCCGTGTGGACAGGCGACAACACAGC
AGAATGGAGCAACTTGAAAATTTCTATCCCAATGTTACTCACTCTCAGCATTACTGGGATCTCTTTTT
GCGGAGCTGACATAGGCGGGTTCATTGGGAATCCAGAGACAGAGCTGCTAGTGCGTTGGTACCAGGCT
GGAGCCTACCAGCCCTTCTTCCGTGGCCATGCACCATGAACACCAAGCGACGAGAGCCCTGAGCTCTT
TGGGGAGGAACACACCCGACTCATCCGAGAACCATCAGAGAGCGCTATGGCCTCCTGCCATAATTGGT
ATTCTCTGTTCTACCATGCACACGTGGCTTCCCAACCTGTCATGAGGCCTCTGTGGGTAGAGTTCCCT
GATGAACTAAAGACTTTTGATATGGAAGATGAATACATGCTGGGGAGTGCATTATTGGTTCATCCAGT
CACAGAACCAAAAGCCACCACAGTTGATGTGTTTCTTCCAGGATCAAATGAGGTCTGTATGCACTATA
AGACATTTGCTCATTGGGAAGGAGGGTGTACTGTAAAGATCCCAGTAGCCTTGGACACTATTCCAGTG
TTTCAGCGAGGTGGAAGTGTGATACCAATAAAGACAACTGTAGGAAAATCCACAGGCTGGATGACTGA
ATCCTCCTATGGACTCCGGGTTGCTCTAAGCACTAAGGGTTCTTCAGTGGGTGAGTTATATCTTGATG
ATGGCCATTCATTCCAATACCTCCACCAGAAGCAATTTTTGCACAGGAAGTTTTCATTCTGTTCCAGT
GTTCTGATCAATAGTTTTGCTGACCAGAGGGGTCATTATCCCAGCAAGTGTGTGGTGGAGAAGATCTT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
GTTCTGATCAATAGTTTTGCTGACCAGAGGGGTCACTATCCAGCAAGTGTGGGTGGAGAAAGATCTAT
GGTCTTAGGCTTCAGGAAGGAGCCATCTTCTGTGACTACCCACTCATCTGATGGTAAAGATCAGCCTG
TGGCTTTTACGTATTGTGCCAAAACATCCATCCTGAGCCTGGAGAAGCTCTCACTCAACATTGCCACT
CTCTGGGGACACAGGCAGTCTGATATTGGCAGATGGAAAAGGAGACCTGAAGTGCCATATCACAGCAA
ACCCATTCAAGGTAGACTTGGTGTCTGAAGAAGAGGTTGTGATTAGCATAAATTCCCTGGGAAAATTA
GTTCTTACACGTTCTTTCTTTGCTGGATCACAAAAGTATGGTGCCGTGTGGACAGGCGACAACACAGC
AGAATGGAGCAACTTGAAAATTTCTATCCCAATGTTACTCACTCTCAGCATTACTGGGATCTCTTTTT
GCGGAGCTGACATAGGCGGGTTCATTGGGAATCCAGAGACAGAGCTGCTAGTGCGTTGGTACCAGGCT
GGAGCCTACCAGCCCTTCTTCCGTGGCCATGCACCATGAACACCAAGCGACGAGAGCCCTGAGCTCTT
TTCTTCATGGGTGAGTTATATCTTGATGATGGCCATTCATTCCAATACCTCCACCAGAAGCAAATTTT
TGCACAGGAAGTTTTCATTCTGTTCAGTGGCTGATCAATAGTTTTGCTGACCAGAGGAAGGTCATTAT
CCCAGCAAGTGTGTGGTGGAGAAGATCTTGGTCTTAGGCTTCAGGAAGGAGCCATCTTCTGTGACTAC
CCACTCATCTGATGGTAAAGATCAGCCTGTGGCTTTTACGTATTGTGCCAAAACATCCATCCTGAGCC
TGGAGAAGCTCTCACTCAACATTGCCACTGACTGGGAGGTCCGCATCATATGACAAAGAACTGCCCCT
GGTGATGTGAGCAGGGACCTGCCTGCCCCTTTCAACCTTTCCCCTCACCTTTTTTGAGATTTTTGCTG
CAATCTGTTTGTCTTCCCTGAATCAAAATAATCTTTCATTCGTCACCATTATACTAATGAACAATAGA
TTTCATGTTTCAAAATTTCAGATTTTACATGTTAAGATGTACTAACAATATTCCTTGTATCAAACATC
TCCTTTTCTCCCTGATACATAGCCCTGAGACATTATAGCGTC

| | | | |
|---|---|---|---|
| dNOV44f, CG55752-04 | | | |
| Protein Sequence | SEQ ID NO: 670 | 912 aa | MW at 104189.1kD |

MEAAVKEEISVEDEAVDKNIFRDCNKIAFYRRQKQWLSKKSTYRALLDSVTTDEDSTRFQIINEASKV
PLLAEIYGIEGNIFRLKINEETPLKPRFEVPDVLTSKPSTVRLISCSGDTGSLILADGKGDLKCHITA
NPFKVDLVSEEEVVISINSLGQLYFEHLQILHKQRAAKENEEETSVDTSQENQEDLGLWEEKFGKFVD
IKANGPSSIGLDFSLHGFEHLYGIPQHAESHQLKNTGDAYRLYNLDVYGYQIYDIGGIYGSVPYLLAH
KLGRTIGIFWLNASETLVEINTEPAVEYTLTQMGPVAAKQKVRSRTHVHWMSESGIIDVFLLTGPTPS
DVFKQYSHLTGTAQANPPLFSLGHQCRWNYEDEQDVKAVDAGFDEHDIPYDAMWLDIEHTEGKRYFTW
DKNRFPNPKRMQELLRSKKRKLVVISDPHIKIEPDYSVYVKAKDQGFFVKNQEGEDFEGVCWPGLSSY
LDFThPKVREWYSSLFAFPVYQGSTDILFLWNDMNEPSVFRGPEQTMQKNAIHHGNWEHRELHNIYGF
YHQMATAEGLIKRSKGKERPFVLTRSFFAGSQKYGAVWTGDNTAEWSNLKISIPMLLTLSITGISFCG
ADIGGFIGNPETELLVRWYQAGAYQPFFRGHATMNTKRREPWLFGEEHTRLIREAIRERYGLLPYWYS
LFYHAHVASQPVMRPLWVEFPDELKTFDMEDEYMLGSALLVHPVTEPKATTVDVFLPGSNEVWYDYKT
FAHWEGGCTVKIPVALDTIPVFQRGGSVIPIKTTVGKSTGWMTESSYGLRVALSTQGSSVGELYLDDG
HSFQYLHQKQFLHRKFSFCSSVLINSFADQRGHYPSKCVVEKILVLGFRKEPSSVTTHSSDGKDQPVA
FTYCAKTSILSLEKLSLNIATDWEVRII

| | | |
|---|---|---|
| NOV44g, CG55752-05 | SEQ ID NO: 671 | 2951 bp |
| DNA Sequence | ORF Start: ATG at 210 | ORF Stop: TGA at 2688 |

TACTGGTTGTAATTTTAGAAAGACACCCAATCGGCTTTTTTAAAAGATCGCCCAGGGCCCTTGTCCTG
AGAGCTGGGAGCTGGTCGGAGTGACAGAGAAGCCATGGAAGCAGCAGTGAAAGAGGAAATAAGTGTTG
AAGATGAAGCTGTAGATAAAAACATTTTCAGAGACTGTAACAAGATCGCATTTTACAGGAAGAAAAGC
GTATTATGTTAATTCTGAACAGGCGTCAGAAACAGTGGCTTTCCAAGAAGTCCACCTATCGGGCATTA
TTGGATTCAGTCACAACAGATGAAGACAGCACCAGGTTCCAAATCATCAATGAAGCAAGTAAGGTTCC
TCTCCTGGCTGAAATTTATGGTATAGAAGGAAACATTTTCAGGCTTAAAATTAACGAAGAGACTCCTC
TAAAACCCAGATTTGAAGTTCCGGATGTCCTCACAAGCAAGCCAAGCACTGTAAGAGCTGCTAAAGAA
AATGAGGAGGAGACATCAGTGGACACCTCTCAGGAAAATCAAGAAGATCTGGGCCTGTGGGAAGAGAA
ATTTGGAAAATTTGTGGATATCACAGCTAATGGCCCTTCTTCTATTGGTTTGGATTTCTCCTTGCATG
GATTTGAGCATCTTTATGGGATCCCACAACATGCAGAATCACACCAACTTAAAAATACTGGAGATGCT
TACCGTCTTTATAACCTGGATGTCTATGGATACCAAATATATGATAAAATGGGCATTTATGGTTCAGT
ACCTTATCTCCTGGCCCACAAACTGGGCAGAACTATAGGTATTTTCTGGCTGAATGCCTCGGAAACAC
TGGTGGAGATCAATACAGAGCCTGCAGTAGAGTACACACTGACCCAGATGGGCCCAGTTGCTGCTAAA
CAAAAGGTCAGATCTCGCACTCATGTGCACTGGATGTCAGAGAGTGGCATCATTGATGTTTTTCTGCT
GACAGGACCTACACCTTCTGATGTCTTCAAACAGTACTCACACCTTACAGGTACGCAAGCCATGCCCC
CTCTTTTCTCTTTGGGATACCACCAGTGCCGCTGGAACTATGAAGATGAGCAGGATGTAAAAGCAGTG
GATGCAGGGTTTGATGAGCATGACATTCCTTATGATGCCATGTGGCTGGACATAGAGCACACTGAGGG
CAAGAGGTACTTCACCTGGGACAAAAACAGATTCCCAAACCCCAAGAGGATGCAAGAGCTGCTCAGGA
GCAAAAAGCGTAAGCTTGTGGTCATCAGTGATCCCCACATCAAGATTGAACCTGACTACTCAGTATAT
GTGAAGGCCAAAGATCAGGGCTTCTTTGTGAAGAATCAGGAAGGGGAAGACTTTGAAGGGGTGTGTTG
GCCAGGTCTCTCCTCTTACCTGGATTTCACCAATCCCAAGGTCAGAGAGTGGTATTCAAGTCTTTTTG
CTTTCCCTGTTTATCAGGGATCTACGGACATCCTCTTCCTTTGGAATGACATGAATGAGCCTTCTGTC
TTTAGAGGGCCAGAGCAAACCATGCAGAAGAATGCCATTCATCATGGCAATTGGGAGCACAGAGAGCT
CCACAACATCTACGGTTTTTATCATCAAATGGCTACTGCAGAAGGACTGATAAAACGATCTAAAGGGA
AGGAGAGACCCTTTGTTCTTACACGTTCTTTCTTTGCTGGATCACAAAAGTATGGTGCCGTGTGGACA
GGCGACAACACAGCAGAATGGAGCAACTTGAAAATTTCTATCCCAATGTTACTCACTCTCAGCATTAC
TGGGATCTCTTTTTGCGGAGCTGACATAGGCGGGTTCATTGGGAATCCAGAGACAGAGCTGCTAGTGC
GTTGGTACCAGGCTGGAGCCTACCAGCCCTTCTTCCGTGGCCATGCCACCATGAACACCAAGCGACGA
GAGCCCTGGCTCTTTGGGGAGGAACACACCCGACTCATCCGAGAAGCCATCAGAGAGCGCTATGGCCT
CCTGCCATATTGGTATTCTCTGTTCTACCATGCACACGTGGCTTCCCAACCTGTCATGAGGCCTCTGT
GGGTAGAGTTCCCTGATGAACTAAAGACTTTTGATATGGAAGATGAATACATGTTAGGGAGTGCATTA
TTGGTTCATCCAGTCACAGAACCAAAAGCCACCACAGTTGATGTGTTTCTTCCAGGATCAAATGAGGT
ATGGTATGACTATAAGACATTTGCTCATTGGGAAGGAGGGTGTACTGTAAAGATCCCAGTAGCCTTGG
ACACTATTCCAGTGTTTCAGCGAGGTGGAAGTGTGATACCAATAAAGACAACTGTAGGAAAATCCACA
GGCTGGATGACTGAATCCTCCTATGGACTCCGGGTTGCTCTAAGCACTCAGGGTTCTTCAGTGGGTGA
GTTATATCTTGATGATGGCCATTCATTCCAATACCTCCACCAGAAGCAATTTTTGCACAGGAAGTTTT
CATTCTGTTCCAGTGTTCTGATCAATAGTTTTGCTGACCAGAGGGGTCATTATCCCAGCAAGTGTGTG
GTGGAGAAGATCTTGGTCTTAGGCTTCAGGAAGGAGCCATCTTCTGTGACTACCCACTCATCTGATGG
TAAAGATCAGCCTGTGGCTTTTACGTATTGTGCCAAAACATCCATCCTGAGCCTGGAGAAGCTCTCAC
TCAACATTGCCACTGACTGGGAGGTCCGCATCATATGACAAAGAACTGCCCCTGGTGATGTGAGCAGG
GACCTGCCTGCCCCTTTCAACGTTTCCCCTCACCTTTTTTGAGATTTTTGCTGCAATCTGTTTGTCTT
CCCTGAATCAAAATAATCTTTCATTCGTCACCATTATACTAATGAACAATAGATTTCATGTTTCAAAA
TTTCAGATTTTACATGTTAAGATGTACTAACAATATTCCTTGTATCAAACATCTCCTTTTCTCCCTGA
TACATAGCCCTGAGACATTATAGCGTC

| | | | |
|---|---|---|---|
| NOV44g, CG55752-05 | | | |
| Protein Sequence | SEQ ID NO: 672 | 826 aa | MW at 94610.4kD |

MLILNRRQKQWLSKKSTYRALLDSVTTDEDSTRFQIINEASKVPLLAEIYGIEGNIFRLKINEETPLK
PRFEVPDVLTSKPSTVRAAKENEEETSVDTSQENQEDLGLWEEKFGKFVDITANGPSSIGLDFSLHGF
EHLYGIPQHESHQLKNTGDAYRLYNLDVYGYQIYDKMGAIYGSVPYLLANKLGRTIGIFWLNASETLV
EINTEPAVEYTLTQMGPVAAKQKVRSRTHVHWMSESGIIDVFLLTGPTPSDVFKQYSHLTGTQANPPL
FSLGYHQCRWNYEDEQDVKAVDAGFDEHDIPYDAMWLDIEHTEGKRYFTWDKNRFPNPKRMQELLRSK
KRKLVVISDPHIKIEPDYSVYVKAKDQGFFVKNQEGEDFEGVCWPGLSSYLDFTNPKVREWYSSLFAF
PVYQGSTDILFLWNDMNEPSVFRGPEQTMQKNAIHHGNWEHRELHNIYGFYHQMATAEGLIKRSKGKE
RPFVLTRSFFAGSQKYGAVWTGDNTAEWSNLKISIPMLLTLSITGISFCGADIGGFIGNPETELLVRW
YQAGAYQPFFRGHATMNTKRREPWLFGEEHTRILREAIRERYGLLPYWYSLFYHAHVASQPVMRPLWV
EFPDELKTFDMEDEYMLGSALLVHPVTEPKATTVDVFLPGSNEVWYDYKTFAHWEGGCTVKIPVALDT
IPVFQRGGSVIPIKTTVGKSTGWMTESSYGLRVALSTQGSSVGELYLDDGHSFQYLHQKQFLHRKFSF
CSSVLINSFADQRGHYPSKCVVEKILVLGFRKEPSSVTTHSSDGKDQPVAFTYCAKTSILSLEKLSLN
IATDWEVRII

| | | |
|---|---|---|
| NOV44h, | | |
| SNP13379656 of | SEQ ID NO: 673 | 2001 bp |
| CG55752-06, | ORF Start: ATG at 1 | ORF Stop: TGA at 1972 |
| DNA Sequence | SNP Pos: 617 | SNP Change: A to G |

ATGGGCATTTATGGTTCAGTACCTTATCTCCTGGCCCACAAACTGGGCAGAACTATAGGTATTTTCTG
GCTGAATGCCTCGGAAACACTGGTGGAGATCAATACAGAGCCTGCAGTAGAGTACACACTGACCCAGA
TGGGCCCAGTTGCTGCTAAACAAAAGGTCAGATCTCGCACTCATGTGCACTGGATGTCAGAGAGTGGC
ATCATTGATGTTTTTCTGCTGACAGGACCTACACCTTCTGATGTCTTCAAACAGTACTCACACCTTAC
AGGTATTCAAGCCATGCCCCCTCTTTTCTCTTTGGGATACCACCAGTGCCGCTGGAACTATGAAGATG
AGCAGGATGTAAAAGCAGTGGATGCAGGGTTTGATGAGCATGACATTCCTTATGATGCCATGTGGCTG
GACATAGAGCACACTGAGGGCAAGAGGTACTTCACCTGGGACAAAAACAGATTCCCAAACCCCAAGAG
GATGCAAGAGCTGCTCAGGAGCAAAAAGCGTAAGGTACTTGTGGTCATCAGTGATCCCCACATCAAGA
TTGATCCTGACTACTCAGTATATGTGAAGGCCAAAGATCAGGGCTTCTTTGTGAAGAATCAGGAAGGG
GAAGGCTTTGAAGGGGTGTGTTGGCCAGGTCTCTCCTCTTACCTGGATTTCACCAATCCCAAGGTCAG
AGAGTGGTATTCAAGTCTTTTTGCTTTCCCTGTTTATCAGGGATCTACGGACATCCTCTTCCTTTGGA
ATGACATGAATGAGCCTTCTGTCTTTAGAGGGCCAGAGCAAACCATGCAGAAGAATGCCATTCATCAT
GGCAATTGGGAGCACAGAGAGCTCCACAACATCTACGGTTTTTATCATCAAATGGCTACTGCAGAAGG
ACTGATAAAACGATCTAAAGGGAAGGAGAGACCCTTTGTTCTTACACGTTCTTTCTTTGCTGGATCAC
AAAAGTATGGTGCCGTGTGGACAGGCGACAACACAGCAGAATGGAGCAACTTGAAAATTTCTATCCCA
ATGTTACTCACTCTCAGCATTACTGGGATCTCTTTTTGCGGAGCTGACATAGGCGGGTTCATTGGGAA
TCCAGAGACAGAGCTGCTAGTGCGTTGGTACCAGGCTGGAGCCTACcAGCCCTTCTTCCGTGGCCATG
CCACCATGAACACCAAGCGACGAGAGCCCTGGCTCTTTGGGGAGGAACACACCCGACTCATCCGAGAA
GCCATCAGAGAGCGCTATGGCCTCCTGCCATATTGGTATTCTCTGTTCTACCATGCACACGTGGCTTC
CCAACCTGTCATGAGGAGGCCTCTGTGGGTAGAGTTCCCTGATGAACTAAAGACTTTTGATATGGAAG
ATGAATACATGCTGGGTTTAGGGAGTGCATTATTGGTTCATCCAGTCACAGAACCAAAAGCCACCACA
GTTGATGTGTTTCTTCCAGGATCAAATGAGGTATGGTATGACTATAAGACATTTGCTCATTGGGAAGG
AGGGTGTACTGTAAAGATCCCAGTAGCCTTGGACACTATTCCAGTGTTTCAGCGAGGTGGAAGTGTGA
TACCAATAAAGACAACTGTAGGAAAATCCACAGGCTGGATGACTGAATCCTCCTATGGACTCCGGGTT
GCTCTAAGCACTGGTTCTTCAGTGGGTGAGTTATATCTTGATGATGGCCATTCATTCCAATACCTCCA
CCAGAAGCAATTTTTGCACAGGAAGTTTTCATTCTGTTCCAGTGTTCTGATCAATTCCAGTITTGCTG
ACCAGAGGGGTCATTATCCCAGCAAGTGTGTGGTGGAGAAGATCTTGGTCTTAGGCTTCAGGAAGGAG
CCATCTTCTGTGACTACCCACTCATCTGATGGTAAAGATCAGCCTGTGGCTTTTACGTATTGTGCCAA
AACATCCATCCTGAGCCTGGAGAAGCTCTCACTCAACATTGCCACTGACTGGGAGGTCCGCATCATAT
GACAAAGAACTGCCCCTGGTGATGTGAGC

| | | | |
|---|---|---|---|
| NOV44h, | | | |
| SNP13379656 of | | | |
| CG55752-06, | SEQ ID NO: 674 | | MW at 75108.9kD |
| Protein Sequence | SNP Pos: 206 | 657 aa | SNP Change: Asp to Gly |

MGIYGSVPYLLAHKLGRTIGIFWLNASETLVEINTEPAVEYTLTQMGPVAAKQKVRSRTHVHWMSESG
IIDVFLLTGPTPSDVFKQYSHLTGIQAMPPLFSLGYHQCRWNYEDEQDVKAVDAGFDEHDIPYDAMWL
DIEHTEGKRYFTWDKNRFPNPKRMQELLRSKKRKVLVVISDPHIKIDPDYSVYVKAKDQGFFVKNQEG
EGFEGVCWPGLSSYLDFTNPKVREWYSSLFAFPVYQGSTDILFLWNDMNEPSVFRGPEQTMQKNAIHH
GNWEHRELHNIYGFYHQMATAEGLIKRSKGKAAERPAFVLTRSQFFAGSQKGTGDNTAEWSNLKISIP
MLLTLSITGISFCGADIGGFIGNPETELLVRWYQAGAYQPFFRGHATMNTKRREPWLFGEEHTRLIRE
AIRERYGLLPYWYSLFYHAHVASQPVMRRPLWVEFPDELKTFDMEDEYMLGLGSALLVHPVTEPKATT
VDVFLPGSNEVWYDYKTFAHWEGGCTVKIPVALDTIPVFQRGGSVIPIKTTVGKSTGWMTESSYGLRV
ALSTGSSVGELYLDDGHSFQYLHQKQFLHRKFSFCSSVLINSSFADQRGHYPSKCVVEKILVLGFRKE
PSSVTTHSSDGKDQPVAFTYCAKTSILSLEKLSLNIATDWEVRII

| | | |
|---|---|---|
| NOV44i, | | |
| SNP13379655 of | SEQ ID NO: 675 | 2001 bp |
| CG55752-06, | ORF Start: ATG at 1 | ORF Stop: TGA at 1972 |
| DNA Sequence | SNP Pos: 1763 | SNP Change: T to C |

| | |
|---|---|
| ATGGGCATTTATGGTTCAGTACCTTATCTCCTGGCCCACAAACTGGGCAGAACTATAGGTATTTTCTG | |
| GCTGAATGCCTCGGAAACACTGGTGGAGATCAATACAGAGCCTGCAGTAGAGTACACACTGACCCAGA | |
| TGGGCCCAGTTGCTGCTAAACAAAAGGTCAGATCTCGCACTCATGTGCACTGGATGTCAGAGAGTGGC | |
| ATCATTGATGTTTTTCTGCTGACAGGACCTACACCTTCTGATGTCTTCAAACAGTACTCACACCTTAC | |
| AGGATTCAAGCCATGCCCCTCTTTTCTCTTTTGGGATACCACCAGTGCCGCTGGAACTATGAAAGATG | |
| AGCAGGATGTAAAAGCAGTGGATGCAGGGTTTGATGAGCATGACATTCCTTATGATGCCATGTAACTG | |
| GACATAGAGCACACTGAGGGCAAGAGGTACTTCACCTGGGACAAAAACAGATTCCCAAACCCCAAGAG | |
| GATGCAAGAGCTGCTCAGGAGCAAAAAGCGTAAGGTACTTGTGGTCATCAGTGATCCCCACATCAAGA | |
| TTGATCCTGACTACTCAGTATATGTGAAGGCCAAAGATCAGGGCTTCTTTGTGAAGAATCAGGAAGGG | |
| GAAGACTTTGAAGGGGTGTGTTGGCCAGGTCTCTCCTCTTACCTGGATTTCACCAATCCCAAGGTCAG | |
| ATGACATGAATGAGCCTTCTGTCTTTAGAGGGCCAGAGCAAACCATGCAGAAGAATGCCATTCATCAT | |
| GGCAATTGGGAGCACAGAGAGCTCCACAACATCTACGGTTTTTATCATCAAATGGCTACTGCAGAAGG | |
| ACTGATAAAACGATCTAAAGGGAAGGAGAGACCCTTTGTTCTTACACGTTCTTTCTTTGCTGGATCAC | |
| AAAAGTATGGTGCCGTGTGGACAGGCGACAACACAGCAGAATGGAGCAACTTGAAAATTTCTATCCCA | |
| ATGTTACTCACTCTCAGCATTACTGGGATCTCTTTTTGCGGAGCTGACATAGGCGGGTTCATTGGGAA | |
| TCCAGAGACAGAGCTGCTAGTGCGTTGGTACCAGGCTGGAGCCTACcAGCCCTTCTTCCGTGGCCATG | |
| CCACCATGAACACCAAGCGACGAGAGCCCTGGCTCTTTGGGGAGGAACACACCCGACTCATCCGAGAA | |
| GCCATCAGAGAGCGCTATGGCCTCCTGCCATATTGGTATTCTCTGTTCTACCATGCACACGTGGCTTC | |
| CCAACCTGTCATGAGGAGGCCTCTGTGGGTAGAGTTCCCTGATGAACTAAAGACTTTTGATATGGAAG | |
| ATGAATACATGCTGGGTTTAGGGAGTGCATTATTGGTTCATCCAGTCACAGAACCAAAAGCCACCACA | |
| GTTGATGTGTTTCTTCCAGGATCAAATGAGGTATGGTATGACTATAAGACATTTGCTCATTGGGAAGG | |
| AGGGTGTACTGTAAAGATCCCAGTAGCCTTGGACACTATTCCAGTGTTTCAGCGAGGTGGAAGTGTGA | |
| TACCAATAAAGACAACTGTAGGAAAATCCACAGGCTGGATGACTGAATCCTCCTATGGACTCCGGGTT | |
| GCTCTAAGCACTGGTTCTTCAGTGGGTGAGTTATATCTTGATGATGGCCATTCATTCCAATACCTCCA | |
| CCAGAAGCAATTTTTGCACAGGAAGTTTTCATTCTGTTCCAGTGTTCTGATCAATTCCAGTTTTGCTG | |
| ACCAGAGGGGTCATTATCCCAGCAAGTGTGTGGTGGAGAAGATCTTGGTCTTAGGCTTCAGTCTGGAG | 44j |
| CCATCTTCTGTGACTACCCACTCATCTGATGGTAAAGATCAGCCTGTGGCTTTTACGTATTGTGCCAA | |
| CCATCTTCTGTGACTACCCACTCATCTGATGGTAAAGATCAGCCTGTGGCTTTTACGTATTGTGCCAA | |
| AACATCCATCCTGAGCCTGGAGAAGCTCTCACTCAACATTGCCACTGACTGGGAGGTCCGCATCATAT | |
| GACAAAGAACTGCCCCTGGTGATGTGAGC | |

| | | | |
|---|---|---|---|
| NOV44i, | | | |
| SNP13379655 of | | | |
| CG55752-06, | SEQ ID NO: 676 | | MW at 75106.8kD |
| Protein Sequence | SNP Pos: 588 | 657 aa | SNP Change: Phe to Ser |

MGIYGSVPYLLAHKLGRTIGIFWLNASETLVEINTEPAVEYTLTQMGPVAAKQKVRSRTHVHWMSESG
IIDVFLLTGPTPSDVFKQYSHLTGIQAMPPLFSLGYHQCRWNYEDEQDVKAVDAGFDEHDIPYDAMWL
DIEHTEGKRYFTWDKNRFPNPKRMQELLRSKKRKVLVVISDPHIKIDPDYSVYVKAKDQGFFVKNQEG
EDFEGVCWPGLSSYLDFTNPKVREWYSSLFAFPVYQGSTDILFLWNDMNEPSVFRGPEQTMQKNAIHH
GNWEHRELHNIYGFYHQMATAEGLIKRSKGKERPFVLTRSFFAGSQKYGAVWTGDNTAEWSNLKISIP
MLLTLSITGISFCGAIGGFIGNPETELLVRWYQAGAYQPFFRGHATMNTKRREPWLFGEAEHTRLIRE
AIRERYGLLPYWYSLFYHAHVASQPVMRRPLWVEFPDELKTFDMEDEYMLGLGSALLVHPVTEPKATT
VDVFLPGSNEVWYDYKTFAHWEGGCTVKIPVALDTIPVFQRGGSVIPIKTTVGKSTGWMTESSYGLRV
ALSTGSSVGELYLDDGHSFQYLHQKQFLHRKFSFCSSVLINSSSADQRGHYPSKCVVEKILVLGFRKE
PSSVTTHSSDGKDQPVAFTYCAKTSILSLEKLSLNIATDWEVRII

| | | |
|---|---|---|
| NOV44j, | | |
| SNP13379654 of | SEQ ID NO: 677 | 2001 bp |
| CG55752-06, | ORF Start: ATG at 1 | ORF Stop: TGA at 1972 |
| DNA Sequence | SNP Pos: 1772 | SNP Change: A to G |

ATGGGCATTTATGGTTCAGTACCTTATCTCCTGGCCCACAAACTGGGCAGAACTATAGGTATTTTCTG
GCTGAATGCCTCGGAAACACTGGTGGAGATCAATACAGAGCCTGCATAGAGTACACACTAGACCCAGA
ATGACATGAATGAGCCTTCTGTCTTTAGAGGGCCAGAGCAAACCATGCAGAAGAATGCCATTCATCAT
GGCAATTGGGAGCACAGAGAGCTCCACAACATCTACGGTTTTTATCATCAAATGGCTACTGCAGAAGG
ACTGATAAAACGATCTAAAGGGAAGGAGAGACCCTTTGTTCTTACACGTTCTTTCTTTGCTGGATCAC
AAAAGTATGGTGCCGTGTGGACAGGCGACAACACAGCAGAATGGAGCAACTTGAAAATTTCTATCCCA
ATGTTACTCACTCTCAGCATTACTGGGATCTCTTTTTGCGGAGCTGACATAGGCGGGTTCATTGGGAA
TCCAGAGACAGAGCTGCTAGTGCGTTGGTACCAGGCTGGAGCCTACcAGCCCTTCTTCCGTGGCCATG
CCACCATGAACACCAAGCGACGAGAGCCCTGGCTCTTTGGGGAGGAACACACCCGACTCATCCGAGAA
GCCATCAGAGAGCGCTATGGCCTCCTGCCATATTGGTATTCTCTGTTCTACCATGCACACGTGGCTTC
CCAACCTGTCATGAGGAGGCCTCTGTGGGTAGAGTTCCCTGATGAACTAAAGACTTTTGATATGGAAG
ATGAATACATGCTGGGTTTAGGGAGTGCATTATTGGTTCATCCAGTCACAGAACCAAAAGCCACCACA
GTTGATGTGTTTCTTCCAGGATCAAATGAGGTATGGTATGACTATAAGACATTTGCTCATTGGGAAGG
AGGGTGTACTGTAAAGATCCCAGTAGCCTTGGACACTATTCCAGTGTTTCAGCGAGGTGGAAGTGTGA
TACCAATAAAGACAACTGTAGGAAAATCCACAGGCTGGATGACTGAATCCTCCTATGGACTCCGGGTT
GCTCTAAGCACTGGTTCTTCAGTGGGTGAGTTATATCTTGATGATGGCCATTCATTCCAATACCTCCA
CCAGAAGCAATTTTTGCACAGGAAGTTTTCATTCTGTTCCAGTGTTCTGATCAATTCCAGTTTTGCTG
ACCAGAGGGGTCATTATCCCAGCAAGTGTGTGGTGGAGAAGATCTTGGTCTTAGGCTTCAGGAAGGAG
CCATCTTCTGTGACTACCCACTCATCTGATGGTAAAGATCAGCCTGTGGCTTTTACGTATTGTGCCAA
TGGGCCCAGTTGCTGCTAAACAAAAGGTCAGATCTCGCACTCATGTGCACTGGATGTCAGAGAGTGGC
ATCATTGATGTTTTTCTGCTGACAGGACCTACACCTTCTGATGTCTTCAAACAGTACTCACACCTTAC
AGGTATTCAAGCCATGCCCCCTCTTTTCTCTTTGGGATACCACCAGTGCCGCTGGAACTATGAAGATG
AGCAGGATGTAAAAGCAGTGGATGCAGGGTTTGATGAGCATGACATTCCTTATGATGCCATGTGGCTG
GACATAGAGCACACTGAGGGCAAGAGGTACTTCACCTGGGACAAAAACAGATTCCCAAACCCCAAGAG
GATGCAAGAGCTGCTCAGGAGCAAAAAGCGTAAGGTACTTGTGGTCATCAGTGATCCCCACATCAAGA
ACCGGAGGGGTCATTATCCCAGCAAGTGTGTGGTGGAGAAGATCTTGGTCTTAGGCTTCAGGAAGGAG
CCATCTTCTGTGACTACCCACTCATCTGATGGTAAAGATCAGCCTGTGGCTTTTACGTATTGTGCCAA
AACATCCATCCTGAGCCTGGAGAAGCTCTCACTCAACATTGCCACTGACTGGGAGGTCCGCATCATAT
GACAAAGAACTGCCCCTGGTGATGTGAGC

| | | | |
|---|---|---|---|
| NOV44j, | | | |
| SNP13379654 of | | | |
| CG55752-06, | SEQ ID NO: 678 | | MW at 75194.9kD |
| Protein Sequence | SNP Pos: 591 | 657 aa | SNP Change: Gln to Arg |

MGIYGSVPYLLAHKLGRTIGIFWLNASETLVEINTEPAVEYTLTQMGPVAAKQKVRSRTHVHWMSESG
IIDVFLLTGPTPSDVFKQYSHLTGIQAMPPLFSLGYHQCRWNYEDEQDVKAVDAGFDEHDIPYDAMWL
DIEHTEGKRYFTWDKNRFPNPKRMQELLRSKKRKVLVVISDPHIKIDPDYSVYVKAKDQGFFVKNQEG
EGFEGVCWPGLSSYLDFTNPKVREWYSSLFAFPVYQGSTDILFLWNDMNEPSVFRGPEQTMQKNAIHH
GNWEHRELHNIYGFYHQMATAEGLIKRSKGKAAERPAFVLTRSQFFAGSQKGTGDNTAEWSNLKISIP
MLLTLSITGISFCGADIGGFIGNPETELLVRWYQAGAYQPFFRGHATMNTKRREPWLFGEEHTRLIRE
AIRERYGLLPYWYSLFYHAHVASQPVMRRPLWVEFPDELKTFDMEDEYMLGLGSALLVHPVTEPKATT
VDVFLPGSNEVWYDYKTFAHWEGGCTVKIPVALDTIPVFQRGGSVIPIKTTVGKSTGWMTESSYGLRV
ALSTGSSVGELYLDDGHSFQYLHQKQFLHRKFSFCSSVLINSSFADRRGHYPSKCVVEKILVLGFRKE
PSSVTTHSSDGKDQPVAFTYCAKTSILSLEKLSLNIATDWEVRII

TABLE 44B

Comparison of the NOV44 protein sequences.

| | |
|---|---|
| NOV44a | ------------------------------------------------------------ |
| NOV44b | ------------------------------------------------------------ |
| NOV44c | MRAAVAGIAFRRRRQKQWLSKKSTYQALLDSVTTDEDSTRFQIINEASKVRRQKQWLSKK |
| NOV44d | MEAAVKEEIS------------------VEDEAVDKN--IFRDCNKIAFYRRQKQWLSKK |
| NOV44e | MEAAVKEEIS------------------VEDEAVDKN--IFRDCNKIAFYRRQKQWLSKK |
| NOV44f | MEAAVKEEIS------------------VEDEAVDKN--IFRDCNKIAFYRRQKQWLSKK |
| NOV44g | ---------------------------------------------MLILNRRQKQWLSKK |
| NOV44a | ------------------------------------------------------------ |
| NOV44b | ------------------------------------------------------------ |
| NOV44c | STYQALLDSVTTDEDSTRFQIINEASKVPLLAEIYGIEGNIFRLKINEETPLKPRFEVPD |
| NOV44d | STYQALLDSVTTDEDSTRFQIINEASKVPLLAEIYGIEGNIFRLKINEETPLKPRFEVPD |
| NOV44e | STYQALLDSVTTDEDSTRFQIINEASKVPLLAEIYGIEGNIFRLKINEETPLKPRFEVPD |
| NOV44f | STYRALLDSVTTDEDSTRFQIINEASKVPLLAEIYGIEGNIFRLKINEETPLKPRFEVPD |
| NOV44g | STYRALLDSVTTDEDSTRFQIINEASKVPLLAEIYGIEGNIFRLKINEETPLKPRFEVPD |
| NOV44a | ------------------------------------------------------------ |
| NOV44b | ------------------------------------------------------------ |
| NOV44c | VLTSKPSTVR-ISCSGDTGSLILADGKGDLKCHITANPFKVDLVSEEEVVISINSLGQLY |
| NOV44d | VLTSKPSTVRLISCSGDTGSLILADGKGDLKCHITANPFKVDLVSEEEVVISINSLGQLY |
| NOV44e | VLTSKPSTVRLISCSGDTGSLILADGKGDLKCHITANPFKVDLVSEEEVVISINSLGQLY |
| NOV44f | VLTSKPSTVRLISCSGDTGSLILADGKGDLKCHITANPFKVDLVSEEEVVISINSLGQLY |
| NOV44g | VLTSKP------------------------------------------------------ |
| NOV44a | ------------------------------------------------------------ |
| NOV44b | ------------------------------------------------------------ |
| NOV44c | FEHGRAPRVSFSDKVNLTLGSIWDKIKNLFSRQGSKDPAEGDGAQPEETPRDGDKPEETQ |
| NOV44d | FEH----------------------LQILHKQRAAKENEE-------ETSVDTS--QENQ |
| NOV44e | FEH----------------------LQILHKQRAAKENEE-------ETSVDTS--QENQ |
| NOV44f | FEH----------------------LQILHKQRAAKENEE-------ETSVDTS--QENQ |
| NOV44g | ---------------------------S--TVRAAKENEE-------ETSVDTS--QENQ |
| NOV44a | ------------------------------------------------------------ |
| NOV44b | ------------------------------------------------------------ |
| NOV44c | GKAEKDEPGAWEETFKTHSDSKPYGPSSIGLDFSLHGFEHLYGIPQHAESHQLKNTGDGD |
| NOV44d | -----EDLGLWEEKFGKFVDIKANGPSSIGLDFSLHGFEHLYGIPQHAESHQLKNTGDGD |
| NOV44e | -----EDLGLWEEKFGKFVDIKANGPSSIGLDFSLHGFEHLYGIPQHAESHQLKNTGDGD |
| NOV44f | -----EDLGLWEEKFGKFVDIKANGPSSIGLDFSLHGFEHLYGIPQHAESHQLKNTG--D |
| NOV44g | -----EDLGLWEEKFGKFVDITANGPSSIGLDFSLHGFEHLYGIPQHAESHQLKNTG--D |
| NOV44a | -----------------MGIYGSVPYLLAHKLGRTIGIFWLNASETLVEINTEPAVEYTL |
| NOV44b | -----------------MGIYGSVPYLLAHKLGRTIGIFWLNASETLVEINTEPAVEYTL |
| NOV44c | AYRLYNLDVYGYQIYDKMGIYGSVPYLLAHKLGRTIGIFWLNASETLVEINTEPAGIVIF |
| NOV44d | AYRLYNLDVYGYQIYDKMGIYGSVPYLLAHKIGRTIGIFWLNASETLVEINTEPAVEYTL |
| NOV44e | AYRLYNLDVYGYQIYDKMGIYGSVPYLLAHKIGRTIGIFWLNASETLVEINTEPAVEYTL |
| NOV44f | AYRLYNLDVYGYQIYDKMGIYGSVPYLLAHKLGRTIGIFWLNASETLVEINTEPAVEYTL |
| NOV44g | AYRLYNLDVYGYQIYDKMGIYGSVPYLLAHKLGRTIGIFWLNASETLVEINTEPAVEYTL |
| NOV44a | TQMGPVAAKQK-VRSRTHVHWMSESGIIDVFLLTGPTPSDVFKQYSHLTGTQAMPPLFSL |
| NOV44b | TQMGPVAAKQK-VRSRTHVHWMSESGIIDVFLLTGPTPSDVFKQYSHLTGIQAMPPLFSL |
| NOV44c | GPVSLIYQSQGDTPLTTHVHWMSESGIIDVFLLTGPTPSDVFKQYSHLTGTQANPPLFSL |
| NOV44d | TQMGPVAAKQK-VRSRTHVHWMSESGIIDVFLLTGPTPSDVFKQYSHLTGTQANPPLFSL |
| NOV44e | TQMGPVAAKQK-VGSRTHVHWMSESGIIDVFLLTGPTPSDVFKQYSHLTGTQANPPLFSL |
| NOV44f | TQMGPVAAKQK-VRSRTHVHWMSESGIIDVFLLTGPTPSDVFKQYSHLTGTQANPPLFSL |
| NOV44g | TQMGPVAAKQK-VRSRTHVHWMSESGIIDVFLLTGPTPSDVFKQYSHLTGTQANPPLFSL |
| NOV44a | GYHQCRNNYEDEQDVKAVDAGFDEHDIPYDANWLDIEHTEGKRYFTWDKNRFPNPKRMQE |
| NOV44b | GYHQCRWNYEDEQDVKAVDAGFDEHDIPYDAMWLDIEHTEGKRYFTWDKNRFPNPKRMQE |
| NOV44c | GYHQCRWNYEDEQDVKAVDAGFDEHDIPYDAIWLDIEHTEGKRYFTWDKNRFPNPKRMQE |
| NOV44d | GYHQCRWNYEDEODVKAVDAGFDEHDIPYDAMWLDIEHTEGKRYFTWDKNRFPNPKRMQE |
| NOV44e | GYHQCRWNYEDEQDVKAVDAGFDEHDIPYDAMWLDIEHTEGKRYFTWDKNRFPNPKRMQE |
| NOV44f | GYHQCRWNYEDEQDVKAVDAGFDEHDIPYDANWLDIEHTEGKRYFTWDKNRFPNPKRMOE |
| NOV44g | GYHQCRWNYEDEQDVKAVDAGFDEHDIPYDAMWLDIEHTEGKRYFTWDKNRFPNPKRMQE |
| NOV44a | LLRSKKR-KLVVISDPHIKIDPDYSVYVKAKDQGFFVKNQEGEDFEGVCWPGLSSYLDFT |
| NOV44b | LLRSKKRKVLVVISDPHIKIDPDYSVYVKAKDQGFFVKNQEGEDFEGVCWPGLSSYLDFT |
| NOV44c | LLRSKKR-KLVVISDPHIKIEPDYSVYVKAKDOGFFVKNQEGEDFEGVCWPGMKSYLDFT |
| NOV44d | LLRSKKR-KLVVISDPHIKIDPDYSVYVKAKDQGFFVKNQEGEDFEGVCWPGLSSYLDFT |
| NOV44e | LLRSKKR-KLVVISDPHIKIDPDYSVYVKAKDQGFFVKNQEGEDFEGVCWPGLSSYLDFT |
| NOV44f | LLRSKKR-KLVVISDPHIKIEPDYSVYVKAKDQGFFVKNQEGEDFEGVCWPGLSSYLDFT |
| NOV44g | LLRSKKR-KLVVISDPHIKIEPDYSVYVKAKDQGFFVKNQEGEDFEGVCWPGLSSThDFT |
| NOV44a | NPKVREWYSSLFAFPVYQGSTDILFLWNDMNEPSVFRGPEQTMQKNAIHHGNWEHRELHN |
| NOV44b | NPKVREWYSSLFAFPVYQGSTDILFLWNDMNEPSVFRGPEQTMQKNAIHHGNWEHRELHN |
| NOV44c | NPKVREWYSSLFAFPVYQGSTDILFLWNDMNEPSVFRGPEQTMQKNAIHHGNWEHRELHN |
| NOV44d | NPKVREWYSSLFAFPVYQGSTDILFLWNDMNEPSVFRGPEQTMQKNAIHHGNWEHRELHN |
| NOV44e | NPKVREWYSSLFAFPVYQGSTDILFLWNDMNEPSVFRGPEQTMQKNAIHHGNWEHRELHN |
| NOV44f | NPKVREWYSSLFAFPVYQGSTDILFLWNDMNEPSVFRGPEQTMQKNAIHHGNWEHRELHN |
| NOV44g | NPKVREWYSSLFAFPVYQGSTDILFLWNDMNEPSVFRGPEQTMQKNAIHHGNWEHRELHN |
| NOV44a | IYGFYHQMATAEGLIKRSKGKERPFVLTRSFFAGSQKYGGAVWTGDNTAEWSNLKISIPM |
| NOV44b | IYGFYHQMATAEGLIKRSKGKERPFVLTRSFFAGSQKYG-AVWTGDNTAEWSNLKISIPM |
| NOV44c | IYGFY--MATAEGLIKRSKGKERPFVLTRSFFAGSQKYG-AVWTGDNTAEWSNLKISIPM |
| NOV44d | IYGFYHQMATAEGLIKRSKGKERPFVLTRSFFAGSQKYG-AVWTGDNTAEWSNLKISIPM |
| NOV44e | IYGFYHQMATAEGLIKRSKGKERPFVLTRSFFAGSQKYG-AVWTGDNTAEWSNLKISIPM |
| NOV44f | IYGFYHQMATAEGLIKRSKGKERPFVLTRSFFAGSQKYG-AVWTGDNTAEWSNLKISIPM |
| NOV44g | IYGFYHQMATAEGLIKRSKGKERPFVLTRSFFAGSQKYG-AVWTGDNTAEWSNLKISIPM |
| NOV44a | LLTLSITGISFCGADIGGFIGNPETELLVRWYQAGAYQPFFRGHATMNTKRREPWLFGEE |
| NOV44b | LLTLSITGISFCGADIGGFIGNPETELLVRWYQAGAYQPFFRGHATMNTKRREPWLFGEE |
| NOV44c | LLTLSITGISFCGADIGGFIGNPETELLVRWYQAGAYQPFFRGHATMNTKRREPWLFGEE |
| NOV44d | LLTLSITGISFCGADIGGFIGNPETELLVRWYQAGAYQPFFRGHATMNTKRREPWLFGEE |
| NOV44e | LLTLSITGISFCGADIGGFIGNPETELLVRWYQAGAYQPFFRGHATMNTKRREPWLFGEE |
| NOV44f | LLTLSITGISFCGADIGGFIGNPETELLVRWYQAGAYQPFFRGHATMNTKRREPWLFGEE |
| NOV44g | LLTLSITGISFCGADIGGFIGNPETELLVRWYQAGAYQPFFRGHATMNTKRREPWLFGEE |
| NOV44a | HTRLIREAIRERYGLLPYWYSLFYHAHVASQPVMR-PLWVEFPDELKTFDMEDEYMLG-- |
| NOV44b | HTRLIREAIRERYGLLPYWYSLFYHAHVASQPVMRRPLWVEFPDELKTFDMEDEYMLGLG |
| NOV44c | HTRLIREAIRERYGLLPYWYSLFYHAHVASQPVMR-PLWVEFPDELKTFDMEDEYMLG-- |
| NOV44d | HTRLIREAIRERYGLLPYWYSLFYHAHVASQPVMR-PLWVEFPDELKTFDMEDEYMLG-- |
| NOV44e | HTRLIREAIRERYGLLPYWYSLFYHAHVASQPVMR-PLWVEFPDELKTFDMEDEYMLG-- |
| NOV44f | HTRLIREAIRERYGLLPYWYSLFYHAHVASQPVMR-PLWVEFPDELKTFDMEDEYMLG-- |
| NOV44g | HTRLIREAIRERYGLLPYWYSLFYHAHVASQPVMR-PLWVEFPDELKTFDMEDEYMLG-- |
| NOV44a | SALLVHPVTEPKATTVDVFLPGSNE-VWYDYKTFAHWEGGCTVKIPVALDTIPVFQRGGS |
| NOV44b | SALLVHPVTEPKATTVDVFLPGSNE-VWYDYKTFAHWEGGCTVKIPVALDTIPVFQRGGS |
| NOV44c | SALLVHPVTEPKATTVDVFLPGSNEVVWYDYKTFAHWEGGCTVKIPVLLQ-IPVFQRGGS |
| NOV44d | SALLVHPVTEPKATTVDVFLPGSNE-VWYDYKTFAHWEGGCTVKIPVALDTIPVFQRGGS |
| NOV44e | SALLVHPVTEPKATTVDVFLPGSNE-VWYDYKTFAHWEGGCTVKIPVALDTIPVFQRGGS |
| NOV44f | SALLVHPVTEPKATTVDVFLPGSNE-VWYDYKTFAHWEGGCTVKIPVALDTIPVFQRGGS |
| NOV44g | SALLVHPVTEPKATTVDVFLPGSNE-VWYDYKTFAHWEGGCTVKIPVALDTIPVFQRGGS |
| NOV44a | VIPIKTTVGKSTGWMTESSYGLRVALSTK-GSSVGELYLDDGHSFQYLHQKQFLHRKFSF |
| NOV44b | VIPIKTTVGKSTGWMTESSYGLRVALST--GSSVGELYLDDGHSFQYLHQKQFLHRKFSF |
| NOV44c | VIPIKTTVGKSTGWMTESSYGLRVALSTKQGSSVGELYLDDGHSFQYLHQKQFLHRKFSF |
| NOV44d | VIPIKTTVGKSTGWMTESSYGLRVALSTK-GSSVGELYLDDGHSFQYLHQKQFLHRKFSF |
| NOV44e | VIPIKTTVGKSTGWMTESSYGLRVALSTK-GSSVGELYLDDGHSFQYLHQKQFLHRKFSF |
| NOV44f | VIPIKTTVGKSTGWMTESSYGLRVALSTQ-GSSVGELYLDDGHSFQYLHQKQFLHRKFSF |
| NOV44g | VIPIKTTVGKSTGWMTESSYGLRVALSTQ-GSSVGELYLDDGHSFQYLHQKQFLHRKFSF |
| NOV44a | CSSVLINSSFADQRGHYPSKCVVEKILVLGFRKEPSSVTTHSSGDGKDQPVAFTYCAKTS |
| NOV44b | CSSVLINSSFADQRGHYPSKCVVEKILVLGFRKEPSSVTTHSS-DGKDQPVAFTYCAKTS |
| NOV44c | CSSVLI-SSPVSQGHLHTPLSMTKALLLFTVSS-PASVTTHSS-DGKDQPVAFTYCAKTS |
| NOV44d | CSSVLI-SSFADQRGHYPSKCVVEKILVLGFRKEPSSVTTHSS-DGKDQPVAFTYCAKTS |
| NOV44e | CSSVLI-SSFADQRGHYPSKCVVEKILVLGFRKEPSSVTTHSS-DGKDQPVAFTYCAKTS |
| NOV44f | CSSVLI-SSFADQRGHYPSKCVVEKILVLGFRKEPSSVTTHSS-DGKDQPVAFTYCAKTS |
| NOV44g | CSSVLI-SSFADQRGHYPSKCVVEKILVLGFRKEPSSVTTHSS-DGKDQPVAFTYCAKTS |
| NOV44a | ILSLEKLSLNIATDWEVRII |
| NOV44b | ILSLEKLSLNIATDWEVRII |
| NOV44c | ILSLEKLSLNIATDWEVRII |
| NOV44d | ILSLEKLSLNIATDWEVRII |
| NOV44e | ILSLEKLSLNIATDWEVRII |
| NOV44f | ILSLEKLSLNIATDWEVRII |
| NOV44g | ILSLEKLSLNIATDWEVRII |
| NOV44a | (SEQ ID NO: 660) |
| NOV44b | (SEQ ID NO: 662) |
| NOV44c | (SEQ ID NO: 664) |
| NOV44d | (SEQ ID NO: 666) |
| NOV44e | (SEQ ID NO: 668) |
| NOV44f | (SEQ ID NO: 670) |
| NOV44g | (SEQ ID NO: 672) |

Further analysis of the NOV44a protein yielded the following properties shown in Table 44C.

TABLE 44C
Protein Sequence Properties NOV44a

| | |
|---|---|
| SignalP analysis: | No Known Signal Sequence Indicated |
| PSORT II analysis: | |

| PSG: | a new signal peptide prediction method | | | |
|---|---|---|---|---|
| | N-region: | length 0; | pos.chg 0; | neg.chg 0 |
| | H-region: | length 13; | peak value | 5.66 |

| | PSG score: 1.26 |
|---|---|
| GvH: | von Heijne's method for signal seq. recognition |
| | GvH score (threshold: −2.1): −7.02 |
| | possible cleavage site: between 51 and 52 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = −2.07 | Transmembrane 337-353 |

| | |
|---|---|
| PERIPHERAL | Likelihood = 3.82 (at 496) |

| | ALOM score: −2.07 (number of TMSs: 1) |
|---|---|
| MTOP: | Prediction of membrane topology (Hartmann et al.) |
| | Center position for calculation: 344 |
| | Charge difference: −2.0 C(−3.0) − N(−1.0) |
| | N >= C: N-terminal side will be inside |

>>> membrane topology: type 2 (cytoplasmic tail 1 to 337)
MITDISC: discrimination of mitochondrial targeting seq

| | | | |
|---|---|---|---|
| R content: | 1 | Hyd Moment (75): | 2.91 |
| Hyd Moment(95): | 0.96 | G content: | 4 |
| D/E content: | 1 | S/T content: | 3 |
| Score: −6.13 | | | |

Gavel: prediction of cleavage sites for mitochondrial preseq

R-2 motif at 27 GRT|IG

NUCDISC: discrimination of nuclear localization signals

pat4: KKRK (5) at 167
pat7: none
bipartite: none
content of basic residues: 10.4%
NLS Score: −0.16

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: MGIYGSV
Prenylation motif: none
memYQRL: transport motif from cell surface to Golgi: none
Tyrosines in the tail: too long tail
Dileucine motif in the tail: found

LL at 10
LL at 74
LL at 163

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

```
30.4%: cytoplasmic
30.4%: mitochondrial
17.4%: Golgi
  8.7%: endoplasmic reticulum
  4.3%: extracellular, including cell wall
  4.3%: nuclear
  4.3%: vesicles of secretory system
```

>> prediction for CG55752-07 is cyt (k = 23)

A search of the NOV44a protein against the Geneseq database, a proprietary database that contains sequences published in patents and patent publication, yielded several homologous proteins shown in Table 44D.

TABLE 44D
Geneseq Results for NOV44a

| | | NOV44a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG69611 | Human NOV11b protein - Homo | 1 . . . 656 | 653/656 (99%) | 0.0 |
| | sapiens, 914 aa. [WO200250277- | 262 . . . 914 | 653/656 (99%) | |
| | A2, 27-JUN-2002] | | | |
| ABG69613 | Human NOV11d protein - Homo | 1 . . . 656 | 651/656 (99%) | 0.0 |
| | sapiens, 912 aa. [WO200250277- | 260 . . . 912 | 653/656 (99%) | |
| | A2, 27-JUN-2002] | | | |
| ABG69612 | Human NOV11c protein - Homo | 1 . . . 656 | 651/656 (99%) | 0.0 |
| | sapiens, 914 aa. [WO200250277- | 262 . . . 914 | 652/656 (99%) | |
| | A2, 27-JUN-2002] | | | |
| AAG79779 | Carbohydrate-associated protein | 1 . . . 652 | 648/652 (99%) | 0.0 |
| | (CHOP)-1 - Homo sapiens, 912 | 262 . . . 910 | 648/652 (99%) | |
| | aa. [WO200297060-A2, 05-DEC- | | | |
| | 2002] | | | |
| ABP52437 | Human carbohydrate metabolism | 1 . . . 656 | 634/656 (96%) | 0.0 |
| | enzyme CME-2 protein SEQ ID | 262 . . . 914 | 636/656 (96%) | |
| | NO: 2 - Homo sapiens, 914 aa. | | | |
| | [WO200261059-A2, 08-AUG- | | | |
| | 2002] | | | |

In a BLAST search of public sequence databases, the NOV44a protein was found to have homology to the proteins shown in the BLASTP data in Table 44E.

TABLE 44E
Public BLASTP Results for NOV44a

| | | NOV44a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IWZ0 | Glucosidase - Homo sapiens | 1 . . . 656 | 653/656 (99%) | 0.0 |
| | (Human), 769 aa (fragment). | 117 . . . 769 | 653/656 (99%) | |
| Q8TET4 | FLJ00088 protein - Homo | 1 . . . 656 | 653/656 (99%) | 0.0 |
| | sapiens (Human), 925 aa | 273 . . . 925 | 653/656 (99%) | |
| | (fragment). | | | |
| Q8IZM5 | Neutral alpha glucosidase C | 1 . . . 656 | 653/656 (99%) | 0.0 |
| | type 2 - Homo sapiens | 262 . . . 914 | 653/656 (99%) | |
| | (Human), 914 aa. | | | |
| AAN74755 | Neutral alpha glucosidase C - | 1 . . . 656 | 652/656 (99%) | 0.0 |
| | synthetic construct, 914 aa. | 262 . . . 914 | 653/656 (99%) | |
| Q8IZM4 | Neutral alpha-glucosidase C | 1 . . . 656 | 652/656 (99%) | 0.0 |
| | type 3 - Homo sapiens (Human), | 262 . . . 914 | 653/656 (99%) | |
| | 914 aa. | | | |

PFam analysis indicates that the NOV44a protein contains the domains shown in the Table 44F.

TABLE 44F
Domain Analysis of NOV44a

| | NOV44a | Identities/Similarities | Expect |
|---|---|---|---|
| Pfam Domain | Match Region | for the Matched Region | Value |
| Glyco_hydro_31 | 1 . . . 616 | 259/856 (30%) | 3.4e−235 |
| | | 517/856 (60%) | |

### Example 45

The NOV45 clone was analyzed, and the nucleotide and encoded polypeptide sequences are shown in Table 45A.

TABLE 45A

NOV45 Sequence Analysis

| | | |
|---|---|---|
| NOV45a, CG55778-03 | SEQ ID NO: 679 | 1752 bp |
| DNA Sequence | ORF Start: ATG at 23 | ORF Stop: TAA at 653 |

GGCGGGGCGGCCGGCGGCGGCCATGGGAGATATCCCAGCCGTGGGCCTCAGCTCCTGGAAGCAGGCTT
CTCCAGGTAAAGTGACCGAGGCAGTGAAAGAGGCCATTGACGCAGGGTACCGGCACTTCGACTGTGCT
TACTTTTACCACAATGAGAGGGAGGTTGGAGCAGGGATCCGTTGCAAGATCAAGGAAGGCGCTGTAAG
ACGGGAGGATCTGTTCATTGCCACTAAGCTGTGGTGCACCTGCCATAAGAAGTCCTTGGTGGAAACAG
CATGCAGAAAGAGTCTCAAGGCCTTGAAGCTGAACTATTTGGACCTCTACCTCATACACTGGCCCATG
GGTTTCAAGCCTCCTCATCCAGAATGGATCATGAGCTGCAGTGAACTTTCCTTCTGCCTCTCACATCC
TCGAGTGCAGGACTTGCCTCTGGACGAGAGCAACATGGTTATTCCCAGTGACACGGACTTCCTGGACA
CGTGGGAGATTTTGATCCGATTTCAAATCCAGAGGAATGTGATAGTGATCCCCGGATCTATCACCCCA
AGTCACATTAAAGAGAATATCCAGGTGTTTGATTTTGAATTAACACAGCACGATATGGATAACATCCT
CAGCCTAAACAAGAATCTCCGACTGGCCATGTTCCCCATGTAAATATGGCTCCTTCTTTTTAAAACAG
AGGGAAGAATATACAGATTGAATGATTGGTGTCTGAATAGAACTAAAAATCACAAAGACTATCCTTTC
CACA

| | | | |
|---|---|---|---|
| NOV45a, GG55778-03 | | | |
| Protein Sequence | SEQ ID NO: 680 | 210 aa | MW at 24136.7kD |

MGDIPAVGLSSWKQASPGKVTEAVKEAIDAGYRHFDCAYFYHNEREVGAGIRCKIKEGAVRREDLFIA
TKLWCTCHKKSLVETACRKSLKALKLNYLDLYLIHWPMGFKPPHPEWIMSCSELSFCLSHPRVQDLPL
DESNNVIPSDTDFLDTWEILIRFQIQRNVIVIPGSITPSHIKENIQVFDFELTQHDMDNILSLNKNLR
LAMFPM

| | | |
|---|---|---|
| NOV45b, CG55778-06 | SEQ ID NO: 681 | 569 bp |
| DNA Sequence | ORF Start: ATG at 1 | ORF Stop: TGA at 550 |

ATGGGAGATATCCCAGCCGTGGGCCTCAGCTCCTGGAAGGCTTCTCCAGGAAAAGTGACCGAGGCAGT
GAAAGAGGCCATTGACGCAGGGTACCGGCACTTCGACTGTGCTTACTTTTACCACAATGAGAGGGAGG
TTGGAGCAGGGATCCGTTGCAAGATCAAGGAAGGCGCTGTAAGACGGGAGGATCTGTTCATTGCCACT
AAGCCTCCTCATCCAGAATGGATCATGAGCTGCAGTGAACTTTCCTTCTGCCTCTCACATCCTCGAGT
GCAGGACTTGCCTCTGGACGAGAGCAACATGGTTATTCCCAGTGACACGGACTTCCTGGACACGTGGG
AGATTTTGATCCGATTTCAAATCCAGAGGAATGTGATAGTGATCCCCGGATCTATCACCCCAAGTCAC
ATTAAAGAGAATATCCAGGTGTTTGATTTTGAATTAACACAGCACGATATGGATAACATCCACAGCCT
AAACAGGAATCTCCGACTGGCCATGTTCCCCATAACTAAAAATCACAAAGACTATCCTTTCCACATAG
AATACTGAGGACGCTTCCCCTTCCT

| | | | |
|---|---|---|---|
| NOV45b, CG55778-06 | | | |
| Protein Sequence | SEQ ID NO: 682 | 183 aa | MW at 21057.81kD |

MGDIPAVGLSSWKASPGKVTEAVKEAIDAGYRHFDCAYFYHNEREVGAGIRCKIKEGAVRREDLFIAT
KPPHPEWIMSCSELSFCLSHPRVQDLPLDESNMVIPSDTDFLDTWEILIRFQIQRNVIVIPGSITPSH
IKENIQVFDFELTQHDMDNILSLNRNLRLAMFPITKNHKDYPFHIEY

| | | |
|---|---|---|
| NOV45c, 275480984 | SEQ ID NO: 683 | 655 bp |
| DNA Sequence | ORF Start: at 2 | ORF Stop: end of sequence |

CACCAGATCTCCCACCATGGGAGATATCCCAGCCGTGGGCCTCAGCTCCTGGAAGCAGGCTTCTCCAG
GGAAAGTGACCGAGGCAGTGAAAGAGGCCATTGACGCAGGGTACCGGCACTTCGACTGTGCTTACTTT
TACCACAATGAGAGGGAGGTTGGAGCAGGGATCCGTTGCAAGATCAAGGAAGGCGCTGTAAGACGGGA
GGATCTGTTCATTGCCACTAAGCTGTGGTGCACCTGCCATAAGAAGTCCTTGGTGGAAACAGCATGCA
GAAAGAGTCTCAAGGCCTTGAAGCTGAACTATTTGGACCTCTACCTCATACACTGGCCCATGGGTTTC
AAGCCTCCTCATCCAGAATGGATCATGAGCTGCAGTGAACTTTCCTTCTGCCTCTCACATCCTCGAGT
GCAGGACTTGCCTCTGGACGAGAGCAACGTGGTTATTCCCAGTGACACGGACTTCCTGGACACGTGGG
AGATTTTGATCCGATTTCAAATCCAGAGGAATGTGATAGTGATCCCCGGATCTATCACCCCAAGTCAC
ATTAAAGAGAATATCCAGGTGTTTGATTTTGAATTAACACAGCACGATATGGATAACATCCTCAGCCT
AAACAGGAACCTCCGACTGGCCATGTTCCCCATGGTCGACGGC

| | | | |
|---|---|---|---|
| NOV45c, 275480984 | | | |
| Protein Sequence | SEQ ID NO: 684 | 218 aa | MW at 24946.5kD |

TRSPTMGDIPAVGLSSWKQASPGKVTEAVKEAIDAGYRHFDCAYFYHNEREVGAGIRCKIKEGAVRRE
DLFIATKLWCTCHKKSLVETACRKSLKALKLNYLDLYLIHWPMGFKPPHPEWIMSCSELSFCLSHPRV
QDLPLDESNVVIPSDTDFLDTWEILIRFQIQRNVIVIPGSITPSHIKENIQVFDFELTQHDMDNILSL
NRNLRLAMFPMVDG

| | | |
|---|---|---|
| NOV45d, CG55778-01 | SEQ ID NO: 685 | 1956 bp |
| DNA Sequence | ORF Start: ATG at 31 | ORF Stop: TGA at 937 |

GGCGGGGCGGCGGGGCGGCCGGCGGCGGCCATGGGAGATATCCCAGCCGTGGGCCTCAGCTCCTGGAA
GCAGGCTTCTCCAGGGAAAGTGACCGAGGCAGTGAAAGAGGCCATTGACGCAGGGTACCGGCACTTCG
ACTGTGCTTACTTTTACCACAATGAGAGGGAGGTTGGAGCAGGGATCCGTTGCAAGATCAAGGAAGGC
GCTGTAAGACGGGAGGATCTGTTCATTGCCACTAAGCTGTGGTGCACCTGCCATAAGAAGTCCTTGGT
GGAAACAGCATGCAGAAAGAGTCTCAAGGCCTTGAAGCTGAACTATTTGGACCTCTACCTCATACACT
GGCCCATGGGTTTCAAGCCTCGAGTGCAGGACTTGCCTCTGGACGAGAGCAACATGGTTATTCCCAGT
GACACGGACTTCCTGGACACGTGGGAGGCCATGGAGGACCTGGTGATCACCGGGCTGGTGAAGAACAT
CGGGGTGTCAAACTTCAACCATGAACAGCTTGAGAGGCTTTTGAATAAGCCTGGGTTGAGGTTCAAGC
CACTAACCAACCAGATTGAGTGCCACCCATATCTTACTCAGAAGAATCTGATCAGTTTTTGCCAATCC
AGAGATGTGTCCGTGACTGCTTACCGTCCTCTTGGTGGCTCTAGTGAGGGGGTTGACCTGATAGACAA
CCCTGTGATCAAGAGGATTGCAAAGGAGCACGGCAAGTCTCCTGCTCAGATTTTGATCCGATTTCAAA
TCCAGAGGAATGTGATAGTGATCCCCGGATCTATCACCCCAAGTCACATTAAAGAGAATATCCAGGTG
TTTGATTTTGAATTAACACAGCACGATATGGATAACATCCTCAGCCTAAACAGGAATCTCCGACTGGC
CATGTTCCCCAGAACTAAAAATCACAAAGACTATCCTTTCCACATAGAATACTGAGGACGCTTCCCCT
TCCT

| | | | |
|---|---|---|---|
| NOV45d, CG55778-01 | | | |
| Protein Sequence | SEQ ID NO: 686 | 302 aa | MW at 34561.5kD |

MGDIPAVGLSSWKQASPGKVTEAVKEAIDAGYRHFDCAYFYHNEREVGAGIRCKIKEGAVRREDLFIA
TKLWCTCHKKSLVETACRKSLKALKLNYLDLYLIHWPMGFKPRVQDLPLDESNMVIPSDTDFLDTWEA
MEDLVITGLVKNIGVSNFNHEQLERLLNKPGLRFKPLTNQIECHPYLTQKNLISFCQSRDVSVTAYRP
LGGSSEGVDLIDNPVIKRIAKEHGKSPAQILIRFQIQRNVIVIPGSITPSHIKENIQVFDFELTQHDM
DNILSLNRNLRLANFPRTKNHKDYPFHIEY

| | | |
|---|---|---|
| NOV45e, CG55778-02 | SEQ ID NO: 687 | 875 bp |
| DNA Sequence | ORF Start: ATG at 23 | ORF Stop: TAA at 776 |

GGCGGGGCGGCCGGCGGCGGCCATGGGAGATATCCCAGCCGTGGGCCTCAGCTCCTGGAAGCAGGCTT
CTCCAGGAAAAGTGACCGAGGCAGTGAAAGAGGCCATTGACGCAGGGTACCGGCACTTCGACTGTGCT
TACTTTTACCACAATGAGAGGGAGGTTGGAGCAGGGATCCGTTGCAAGATCAAGGAAGGCGCTGTAAG
ACGGGAGGATCTGTTCATTGCCACTAAGCTGTGGTGCACCTGCCATAAGAAGTCCTTGGTGGAAACAG
CATGCAGAAAGGGTCTCAAGGCCTTGAAGCTGAACTATTTGGACCTCTACCTCATACACTGGCCCATG
GGTTTCAAGCCTCCTCATCCAGAATGGATCATGAGCTGCAGTGAACTTTCCTTCTGCCTCTCACATCC
TCGAGTGCAGGACTTGCCTCTGGACGAGAGCAACATGGTTATTCCCAGTGACACGGACTTCCTGGACA
CGTGGGAGGCCATGGAGGACCTGGTGATCACCGGGCTGGTGAAGAACATCGGGGTGTCAAACTTCAAC
CATGAACAGCTTGAGAGGCTTTTGAATAAGCCTGGGTTGAGGTTCAAGCCACTAACCAACCAGATTTT
GATCCGATTTCAAATCCAGAGGAATGTGATAGTGATCCCCGGATCTATCACCCCAAGTCACATTAAAG
AGAATATCCAGGTGTTTGATTTTGAATTAACACAGCACGATATGGATAACATCCTCAGCCTAAACAGG
AATCTCCGACTGGCCATGTTCCCCATGTAAATATGGCTCCTTCTTTTTAAAACAGAGGGAAGAATATA
CAGATTGAATGATTGGTGTCTGAATAGAACTAAAAATCACAAAGACTATCCTTTCCACA

| | | | |
|---|---|---|---|
| NOV45e, GG55778-02 | | | |
| Protein Sequence | SEQ ID NO: 688 | 251 aa | MW at 28765.1kD |

MGDIPAVGLSSWKQASPGKVTEAVKEAIDAGYRHFDCAYFYHNEREVGAGIRCKIKEGAVRREDLFIA
TKLWCTCHKKSLVETACRKGLKALKLNYLDLYLIHWPMGFKPPHPEWIMSCSELSFCLSHPRVQDLPL
DESNMVIPSDTDFLDTWEAMEDLVITGLVKNIGVSNFNHEQLERLLNKPGLRFKPLThQILIRFQIQR
NVIVIPGSITPSHIKENIQVFDFELTQHDMDNILSLNRNLRLAMFPM

| | | |
|---|---|---|
| NOV45f, GG55778-04 | SEQ ID NO: 689 | 785 bp |
| DNA Sequence | ORF Start: ATG at 31 | ORF Stop: TGA at 766 |

GGCGGGGCGGCGGGGCGGCCGGCGGCGGCCATGGGAGATATCCCAGCCGTGGGCCTCAGCTCCTGGAA
GCAGGCTTCTCCAGGGAAAGTGACCGAGGCAGTGAAAGAGGCCATTGACGCAGGGTACCGGCACTTCG
ACTGTGCTTACTTTTACCACAATGAGAGGGAGGTTGGAGCAGGGATCCGTTGCAAGATCAAGGAAGGC
GCTGTAAGACGGGAGGATCTGTTCATTGCCACTAAGCTGTGGTGCACCTGCCATAAGAAGTCCTTGGT
GGAAACAGCATGCAGAAAGAGTCTCAAGGCCTTGAAGCTGAACTATTTGGACCTCTACCTCATACACT
GGCCCATGGGTTTCAAGCCTCGAGTGCAGGACTTGCCTCTGGACGAGAGCAACATGGTTATTCCCAGT
GACACGGACTTCCTGGACACGTGGGAGGCCATGGAGGACCTGGTGATCACCGGGCTGGTGAAGAACAT
CGGGGTGTCAAACTTCAACCATGAACAGCTTGAGAGGCTTTTGAATAAGCCTGGGTTGAGGTTCAAGC
CACTAACCAACCAGATTTTGATCCGATTTCAAATCCAGAGGAATGTGATAGTGATCCCCGGATCTATC
ACCCCAAGTCACATTAAAGAGAATATCCAGGTGTTTGATTTTGAATTAACACAGCACGATATGGATAA
CATCCTCAGCCTAAACAGGAATCTCCGACTGGCCATGTTCCCCAGAACTAAAAATCACAAAGACTATC
CTTTCCACATAGAATACTGAGGACGCTTCCCCTTCCT

| | | | |
|---|---|---|---|
| NOV45f, CG55778-04 | | | |
| Protein Sequence | SEQ ID NO: 690 | 245 aa | MW at 28311.4kD |

MGDIPAVGLSSWKQASPGKVTEAVKEAIDAGYRHFDCAYFYHNEREVGAGIRCKIKEGAVRREDLFIA
TKLWCTCHKKSLVETACRKSLKALKLNYLDLYLIHWPMGFKPRVQDLPLDESNNVIPSDTDFLDTWEA
MEDLVITGLVKNIGVSNFNHEQLERLLNKPGLRFKPLTNQILIRFQIQRNVIVIPGSITPSHIKENIQ
VFDFELTQHDMDNILSLNRNLRLANFPRTKNHKDYPFHIEY

| | | |
|---|---|---|
| NOV45g, CG55778-05 | SEQ ID NO: 691 | 937 bp |
| DNA Sequence | ORF Start: ATG at 31 | ORF Stop: TAA at 838 |

GGCGGGGCGGCGGGGCGGCCGGCGGCGGCCATGGGAGATATCCCAGCCGTGGGCCTCAGCTCCTGGAA
GCAGGCTTCTCCAGGGAAAGTGACCGAGGCAGTGAAAGAGGCCATTGACGCAGGGTACCGGCACTTCG
ACTGTGCTTACTTTTACCACAATGAGAGGGAGGTTGGAGCAGGGATCCGTTGCAAGATCAAGGAAGGC
GCTGTAAGACGGGAGGATCTGTTCATTGCCACTAAGCCTCCTCATCCAGAATGGATCATGAGCTGCAG
TGAACTTTCCTTCTGCCTCTCACATCCTCGAGTGCAGGACTTGCCTCTGGACGAGAGCAACATGGTTA
TTCCCAGTGACACGGACTTCCTGGACACGTGGGAGGCCATGGAGGACCTGGTGATCACCGGGCTGGTG
AAGAACATCGGGGTGTCAAACTTCAACCATGAACAGCTTGAGAGGCTTTTGAATAAGCCTGGGTTGAG
GTTCAAGCCACTAACCAACCAGATTGAGTGCCACCCATATCTTACTCAGAAGAATCTGATCAGTTTTT
GCCAATCCAGAGATGTGTCCGTGACTGCTTACCGTCCTCTTGGTGGCTCGTGTGAGGGGGTTGACCTG
ATAGACAACCCTGTGATCAAGAGGATTGCAAAGGAGCACGGCAAGTCTCCTGCTCAGATTTTGATCCG
ATTTCAAATCCAGAGGAATGTGATAGTGATCCCCGGATCTATCACCCCAAGTCACATTAAAGAGAATA
TCCAGGTGTTTGATTTTGAATTAACACAGCACGATATGGATAACATCCTCAGCCTAAACAGGAATCTC
CGACTGGCCATGTTCCCCATGTAAATATGGCTCCTTCTTTTTAAAACAGAGGGAAGAATATACAGATT
GAATGATTGGTGTCTGAATAGAACTAAAAATCACAAAGACTATCCTTTCCACA

| | | | |
|---|---|---|---|
| NOV45g, CG55778-05 | | | |
| Protein Sequence | SEQ ID NO: 692 | 269 aa | MW at 30426.6kD |

MGDIPAVGLSSWKQASPGKVTEAVKEAIDAGYRHFDCAYFYHNEREVGAGIRCKIKEGAVRREDLFIA
TKPPHPEWIMSCSELSFCLSHPRVQDLPLDESNMVIPSDTDFLDTWEANEDLVITGLVKNIGVSNFNH
EQLERLLNKPGLRFKPLTNQIECHPYLTQKNLISFCQSRDVSVTAYRPLGGSCEGVDLIDNPVIKRIA
KEHGKSPAQILIRFQIQRNVIVIPGSITPSHIKENIQVFDFELTQHDMDNILSLNRNLRLANFPM

| | | |
|---|---|---|
| NOV45h, CG55778-07 | SEQ ID NO: 693 | 1527 bp |
| DNA Sequence | ORF Start: ATG at 21 | ORF Stop: TGA at 981 |

CGGGGCGGCCGGCGGCGGCCATGGGAGATATCCCAGCCGTGGGCCTCAGCTCCTGGAAGGCTTCTCCA
GGGAAAGTGACCGAGGCAGTGAAAGAGGCCATTGACGCAGGGTACCGGCACTTCGACTGTGCTTACTT
TTACCACAATGAGAGGGAGGTTGGAGCAGGGATCCGTTGCAAGATCAAGGAAGGCGCTGTAAGACGGG
AGGATCTGTTCATTGCCACTAAGCTGTGGTGCACCTGCCATAAGAAGTCCTTGGTGGAAACAGCATGC
AGAAAGAGTCTCAAGGCCTTGAAGCTGAACTATTTGGACCTCTACCTCATACACTGGCCCATGGGTTT
CAAGCCTCCTCATCCAGAATGGATCATGAGCTGCAGTGAACTTTCCTTCTGCCTCTCACATCCTCGAG
TGCAGGACTTGCCTCTGGACGAGAGCAACATGGTTATTCCCAGTGACACGGACTTCCTGGACACGTGG
GAGGCCATGGAGGACCTGGTGATCACCGGGCTGGTGAAGAACATCGGGGTGTCAAACTTCAACCATGA
ACAGCTTGAGAGGCTTTTGAATAAGCCTGGGTTGAGGTTCAAGCCACTAACCAACCAGATTGAGTGCC
ACCCATATCTTACTCAGAAGAATCTGATCAGTTTTTGCCAATCCAGAGATGTGTCCGTGACTGCTTAC
CGTCCTCTTGGTGGCTCGTGTGAGGGGGTTGACCTGATAGACAACCCTGTGATCAAGAGGATTGCAAA
GGAGCACGGCAAGTCTCCTGCTCAGATTTTGATCCGATTTCAAATCCAGAGGAATGTGATAGTGATCC
CCGGATCTATCACCCCAAGTCACATTAAAGAGAATATCCAGGTGTTTGATTTTGAATTAACACAGCAC
GATATGGATAACATCCTCAGCCTAAACAGGAATCTCCGACTGGCCATGTTCCCCATAACTAAAAATCA
CAATGACTATCCTTTCCACATAGAATACTGAGGACGCTTCCCCTTCCTTGTTTCTGCTCAGCCCAGAT
GCACAGACACTATTGGCAATGTTGACCCTCCTCTGTCATCACAGCGCCAGGGCAGCTGTGCCTGGGAC
AGGAGCCACACAGTCAGAGGGGGATGTAAGAGCCACCTTCTCTGACAAATCTGGAGAATTGAGTGTGT
TCTAAGTGAAGGCAATGGGGTTTCTCCAAGACAGCCTGTGTGGCCTCTACTCTGAACAAATACACTGA
TGAGTCATCAGTGAAATTTGCCTTCACATTTTAAGAAAACTTTATCTTATGGAGTTATTTAAGCCATC
TACAGAGCTGAGGAAACAGTGTAATGTGTCTCTGCCCCATTGCGCAGCTCCACCCATTGTGCCCCAGG
CCAGCCCGCGTCACCTACACTTCCTTCTGTGCCCTGCCAGTGACCCCCAGGTTATTCTAAAGCAGAGT
CCTTCCCTTCCCCCAGTGAGAAGGAAAATGGGATAAGTCTGGGACACTGTTTCAGTTCAATAAAGAGG
CTTTTTTCTTCCTTAAAAAAAAAAAAAAAAA

| | | | |
|---|---|---|---|
| NOV45h, CG55778-07 | | | |
| Protein Sequence | SEQ ID NO: 694 | 320 aa | MW at 36574.8kD |

MGDIPAVGLSSWKASPGKVTEAVKEAIDAGYRHFDCAYFYHNEREVGAGIRCKIKEGAVRREDLFIAT
KLWCTCHKKSLVETACRKSLKALKLNYLDLYLIHWPMGFKPPHPEWIMSCSELSFCLSHPRVQDLPLD
ESNMVIPSDTDFLDTWEAMEDLVITGLVKNIGVSNFNHEQLERLLNKPGLRFKPLTNQIECHPYLTQK
NLISFCQSRDVSVTAYRPLGGSCEGVDLIDNPVIKRIAKEHGKSPAQILIRFQIQRNVIVIPGSITPS
HIKENIQVFDFELTQHDMDNILSLNRNLRLANFPITKNHNDYPFHIEY

| | | |
|---|---|---|
| NOV45i, CG55778-08 | SEQ ID NO: 695 | 1680 bp |
| DNA Sequence | ORF Start: ATG at 101 | ORF Stop: TAA at 1022 |

GGCACGAGGACGGCGGGTCGCCAGCGCCTCAGTAGCTCGCGCGGTGCCTGTCGGTAGTCGCGTGCGGG
GCGGCGGGGCGGCGGGGCGGCCGGCGGCGGCCATGGGAGATATCCCAGCCGTGGGCCTCAGCTCCTGG
AAGGCTTCTCCAGGGAAAGTGACCGAGGCAGTGAAAGAGGCCATTGACGCAGGGTACCGGCACTTCGA
CTGTGCTTACTTTTACCACAATGAGAGGGAGGTTGGAGCAGGGATCCGTTGCAAGATCAAGGAAGGCG
CTGTAAGACGGGAGGATCTGTTCATTGCCACTAAGCTGTGGTGCACGTGCCATAAGAAGTCCTTGGTG
GAAACAGCATGCAGAAAGAGTCTCAAGGCCTTGAAGCTGAACTATTTGGACCTCTACCTCATACACTG
GCCCATGGGTTTCAAGCCTCCTCATCCAGAATGGATCATGAGCTGCAGTGAACTTTCCTTCTGCCTCT
CACATCCTCGAGTGCAGGACTTGCCTCTGGACGAGAGCAACATGGTTATTCCCAGTGACACGGACTTC
CTGGACACGTGGGAGGCCATGGAGGACCTGGTGATCACCGGGCTGGTGAAGAACATCGGGGTGTCAAA
CTTCAACCATGAACAGCTTGAGAGGCTTTTGAATAAGCCTGGGTTGAGGTTCAAGCCACTAACCAACC
AGATTGAGTGCCACCCATATCTTACTCAGAAGAATCTGATCAGTTTTTGCCAATCCAGAGATGTGTCC
GTGACTGCTTACCGTCCTCTTGGTGGCTCGTGTGAGGGGGTTGACCTGATAGACAACCCTGTGATCAA
GAGGATTGCAAAGGAGCACGGCAAGTCTCCTGCTCAGATTTTGATCCGATTTCAAATCCAGAGGAATG
TGATAGTGATCCCCGGATCTATCACCCCAAGTCACATTAAAGAGAATATCCAGGTGTTTGATTTTGAA
TTAACACAGCACGATATGGATAACATCCTCAGCCTAAACAGGAATCTCCGACTGGCCATGTTCCCCAT
GTAAATATGGCTCCTTCTTTTTAAAACAGAGGGAAGAATATACAGATTGAATGATTGGTGTCTGAATA
GAACTAAAAATCACAAAGACTATCCTTTCCACATAGAATACTGAGGACGCTTCCCCTTCCTTGTTTCT
GCTCAGCCCAGATGCACAGACACTATTGGCAATGTTGACCCTCCTCTGTCATCACAGCGCCAGGGCAG
CTGTGCCTGGGACAGGAGCCACACAGTCAGAGGGGGATGTAAGAGCCACCTTCTCTGACAAATCTGGA
GAATTGAGTGTGTTCTAAGTGAAGGCAATGGGGTTTCTCCAAGACAGCCTGTGTGGCCTCTACTCTGA
ACAAATACACTGATGAGTCATCAGTGAAATTTGCCTTCACATTTTAAGAAAACTTTATCTTATGGAGT
TATTTAAGCCATCTACAGAGCTGAGGAAACAGTGTAATGTGTCTCTGCCCCATTGCGCAGCTCCACCC
ATTGTGCCCCAGGCCAGCCCGCGTCACCTACACTTCCTTCTGTGCCCTGCCAGTGACCCCCAGGTTAT
TCTAAAGCAGAGTCCTTCCCTTCCCCCAGTGAGAAGGAAAATGGGATAAGTCTGGGACACTGTTTCAG
TTCAATAAGAGGCTTTTTTCTTCCTTAAAAAAAAAAAAAAAAAAAAAA

| | | | |
|---|---|---|---|
| NOV45i, CG55778-08 | | | |
| Protein Sequence | SEQ ID NO: 696 | 307 aa | MW at 34933.0kD |

MGDIPAVGLSSWKASPGKVTEAVKEAIDAGYRHFDCAYFYHNEREVGAGIRCKIKEGAVRREDLFIAT
KLWCTCHKKSLVETACRKSLKALKLNYLDLYLIHWPMGFKPPHPEWIMSCSELSFCLSHPRVQDLPLD
ESNMVIPSDTDFLDTWEAMEDLVITGLVKNIGVSNFNHEQLERLLNKPGLRFKPLTNQIECHPYLTQK
LISFCQSRDVSVTAYRPLGGSCEGVDLIDNPAVIKRIAKEHGKSPAQILIRFQIQRNVIVIPGSITPS
HIKENIQVFDFELTQHDMDNILSLNRNLRLAMFPM

| | | |
|---|---|---|
| NOV45j, | | |
| SNP13375813 of | SEQ ID NO: 697 | 752 bp |
| CG55778-03, | ORF Start: ATG at 23 | ORF Stop: TAA at 653 |
| DNA Sequence SNP Pos: 624 | SNP Change: A to G | |

GGCGGGGCGGCCGGCGGCGGCCATGGGAGATATCCCAGCCGTGGGCCTCAGCTCCTGGAAGCAGGCTT
CTCCAGGTAAAGTGACCGAGGCAGTGAAAGAGGCCATTGACGCAGGGTACCGGCACTTCGACTGTGCT
TACTTTTACCACAATGAGAGGGAGGTTGGAGCAGGGATCCGTTGCAAGATCAAGGAAGGCGCTGTAAG
ACGGGAGGATCTGTTCATTGCCACTAAGCTGTGGTGCACCTGCCATAAGAAGTCCTTGGTGGAAACAG
CATGCAGAAAGAGTCTCAAGGCCTTGAAGCTGAACTATTTGGACCTCTACCTCATACACTGGCCCATG
GGTTTCAAGCCTCCTCATCCAGAATGGATCATGAGCTGCAGTGAACTTTCCTTCTGCCTCTCACATCC
TCGAGTGCAGGACTTGCCTCTGGACGAGAGCAACATGGTTATTCCCAGTGACACGGACTTCCTGGACA
CGTGGGAGATTTTGATCCGATTTCAAATCCAGAGGAATGTGATAGTGATCCCCGGATCTATCACCCCA
AGTCACATTAAAGAGAATATCCAGGTGTTTGATTTTGAATTAACACAGCACGATATGGATAACATCCT
CAGCCTAAACAGGAATCTCCGACTGGCCATGTTCCCCATGTAAATATGGCTCCTTCTTTTTAAAACAG
AGGGAAGAATATACAGATTGAATGATTGGTGTCTGAATAGAACTAAAAATCACAAAGACTATCCTTTC
CACA

| | | | |
|---|---|---|---|
| NOV45j, | | | |
| SNP13375813 of | | | |
| CG55778-03, | SEQ ID NO: 698 | | MW at24164.7kD |
| Protein Sequence SNP Pos: 201 | 210 aa | SNP Change: Lys to Arg | |

MGDIPAVGLSSWKQASPGKVTEAVKEAIDAGYRHFDCAYFYHNEREVGAGIRCKIKEGAVRREDLFIA
TKLWCTCHKKSLVETACRKSLKALKLNYLDLYLIHWPMGFKPPHPEWIMSCSELSFCLSHPRVQDLPL
DESNMVIPSDTDFLDTWEILIRFQIQRNVIVIPGSITPSHIKENIQVFDFELTQHDMDNILSLNRNLR
LAMFPM

A ClustalW comparison of the above protein sequences yields the following sequence alignment shown in Table 45B.

TABLE 45B

Comparison of the NOV45 protein sequences.

| | |
|---|---|
| NOV45a | -----MGDIPAVGLSSWKQASPGKVTEAVKEAIDAGYRHFDCAYFYHNEREVGAGIRCKI |
| NOV45b | -----MGDIPAVGLSSWKAS-PGKVTEAVKEAIDAGYRHFDCAYFYHNEREVGAGIRCKI |
| NOV45c | TRSPTMGDIPAVGLSSWKQASPGKVTEAVKEAIDAGYRHFDCAYFYHNEREVGAGIRCKI |
| NOV45d | -----MGDIPAVGLSSWKQASPGKVTEAVKEAIDAGYRHFDCAYFYHNEREVGAGIRCKI |
| NOV45e | -----MGDIPAVGLSSWKQASPGKVTEAVKEAIDAGYRHFDCAYFYHNEREVGAGIRCKI |
| NOV45f | -----MGDIPAVGLSSWKQASPGKVTEAVKEAIDAGYRHFDCAYFYHNEREVGAGIRCKI |
| NOV45g | -----MGDIPAVGLSSWKQASPGKVTEAVKEAIDAGYRJHFDCAYFYHNERVGAGIRCKI |
| NOV45h | -----MGDIPAVGLSSWKAS-PGKVTEAVKEAIDAGYRHFDCAYFYHNEREVGAGIRCKI |
| NOV45i | -----MGDIPAVGLSSWKAS-PGKVTEAVKEAIDAGYRHFDCAYFYHNEREVGAGIRCKI |
| NOV45a | KEGAVRREDLFIATKLWCTCHKKSLVETACRKSLKALKLNYLDLYLIHWPMGFKPPHPEW |
| NOV45b | KEGAVRREDLFIAT---------------------------------------KPPHPEW |
| NOV45c | KEGAVRREDLFIATKLWCTCHKKSLVETACRKSLKALKLNYLDLYLIHWPMGFKPPHPEW |
| NOV45d | KEGAVRREDLFIATKLWCTCHKKSLVETACR-------------------KSLKALKLNY |
| NOV45e | KEGAVRREDLFIATKLWCTCHKKSLVETACRKGLKALKLNYLDLYLIHWPMGFKPPHPEW |
| NOV45f | KEGAVRREDLFIATKLWCTCHKKSLVETACR-------------------KSLKALKLNY |
| NOV45g | KEGAVRREDLFIAT---------------------------------------KPPHPEW |
| NOV45h | KEGAVRREDLFIATKLWCTCHKKSLVETACRKSLKALKLNYLDLYLIHWPMGFKPPHPEW |
| NOV45i | KEGAVRREDLFIATKLWCTCHKKSLVETACRKSLKALKLNYLDLYLIHWPMGFKPPHPEW |
| NOV45a | IMSCSELSFCLSHPRVQDLPLDESNMVIPSDTDFLDTWE--------------------- |
| NOV45b | IMSCSELSFCLSHPRVQDLPLDESNNVIPSDTDFLDTWE--------------------- |
| NOV45c | IMSCSELSFCLSHPRVQDLPLDESNVVIPSDTDFLDTWE--------------------- |
| NOV45d | LDLYLIHWPMGFKPRVQDLPLDESNMVIPSDTDFLDTWEAMEDLVITGLVKNIGVSNFNH |
| NOV45e | IMSCSELSFCLSHPRVQDLPLDESNMVIPSDTDFLDTWEAMEDLVITGLVKNIGVSNFNH |
| NOV45f | LDLYLIHWPMGFKPRVQDLPLDESNNVIPSDTDFLDTWEAMEDLVITGLVKNIGVSNFNH |
| NOV45g | IMSCSELSFCLSHPRVQDLPLDESNMVIPSDTDFLDTWEANEDLVITGLVKNIGVSNFNH |
| NOV45h | IMSCSELSFCLSHPRVQDLPLDESNNVIPSDTDFLDTWEAMEDLVITGLVKNIGVSNFNH |
| NOV45i | IMSCSELSFCLSHPRVQDLPLDESNNVIPSDTDFLDTWEAMEDLVITGLVKNIGVSNFNH |
| NOV45a | ------------------------------------------------------------ |
| NOV45b | ---------------------------------------------------------IL- |
| NOV45c | ------------------------------------------------------------ |
| NOV45d | EQLERLLNKPGLRFKPLTNQIECHPYLTQKNLISFCQSRDVSVTAYRPLGGSSEGVDLID |
| NOV45e | EQLERLLNKPGLRFKPLTN----------------------------------------- |
| NOV45f | EQLERLLNKPGLRFKPLT------------------------------------------ |
| NOV45g | EQLERLLNKPGLRFKPLTNQIECHPYLTQKNLISFCQSRDVSVTAYRPLGGSCEGVDLID |
| NOV45h | EQLERLLNKPGLRFKPLTNQIECHPYLTQKNLISFCQSRDVSVTAYRPLGGSCEGVDLID |
| NOV45i | EQLERLLNKPGLRFKPLTNQIECHPYLTQKNLISFCQSRDVSVTAYRPLGGSCEGVDLID |
| NOV45a | -----------------ILIRFQIQRNVIVIPGSITPSHIKENIQVFDFELTQHDMDNIL |
| NOV45b | ---------------I--LIRFQIQRNVIVIPGSITPSHIKENIQVFDFELTQHDMDNIL |
| NOV45c | -----------------ILIRFQIQRNVIVIPGSITPSHIKENIQVFDFELTQHDMDNIL |
| NOV45d | NPVIKRIAKEHGKSPAQILIRFQIQRNVIVIPGSITPSHIKENIQVFDFELTQHDMDNIL |
| NOV45e | ----------------QILIRFQIQRNVIVIPGSITPSHIKENIQVFDFELTQHDMDNIL |
| NOV45f | --------N-------QILIRFQIQRNVIVIPGSITPSHIKENIQVFDFELTQHDMDNIL |
| NOV45g | NPVIKRIAKEHGKSPAQILIRFQIQRNVIVIPGSITPSHIKENIQVFDFELTQHDMDNIL |
| NOV45h | NPVIKRIAKEHGKSPAQILIRFQIQRNVIVIPGSITPSHIKENIQVFDFELTQHDMDNIL |
| NOV45i | NPVIKRIAKEHGKSPAQILIRFQIQRNVIVIPGSITPSHIKENIQVFDFELTQHDMDNIL |
| NOV45a | SLNKNLRLAMFPM------------- |
| NOV45b | SLNRNLRLAMFPITKNHKDYPFHIEY |
| NOV45c | SLNRNLRLANFPMVDG---------- |
| NOV45d | SLNRNLRLAMFPRTKNHKDYPFHIEY |
| NOV45e | SLNRNLRLANFPM------------- |
| NOV45f | SLNRNLRLAMFPRTKNHKDYPFHIEY |
| NOV45g | SLNRNLRLAMFPM------------- |
| NOV45h | SLNRNLRLAMFPITKNHNDYPFHIEY |
| NOV45i | SLNRNLRLAMFPM------------- |
| NOV45a | (SEQ ID NO: 680) |
| NOV45b | (SEQ ID NO: 682) |
| NOV45C | (SEQ ID NO: 684) |
| NOV45d | (SEQ ID NO: 686) |
| NOV4Se | (SEQ ID NO: 688) |
| NOV45f | (SEQ ID NO: 690) |
| NOV45g | (SEQ ID NO: 692) |
| NOV45h | (SEQ ID NO: 694) |
| NOV45i | (SEQ ID NO: 696) |

Further analysis of the NOV45a protein yielded the following properties shown in Table 45C.

TABLE 45C
Protein Sequence Properties NOV45a

| | |
|---|---|
| SignalP analysis: | No Known Signal Sequence Indicated |
| PSORT II analysis: | |

| PSG: | a new signal peptide prediction method | | | |
|---|---|---|---|---|
| | N-region: | length 3; | pos.chg 0; | neg.chg 1 |
| | H-region: | length 9; | peak value | 0.00 |

| | PSG score: −4.40 |
|---|---|
| GvH: | von Heijne's method for signal seq. recognition |
| | GvH score (threshold: −2.1): −6.56 |
| | possible cleavage site: between 18 and 19 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 6.21 (at 155) |
| | ALOM score: 6.21 (number of TMSs: 0) |

MITDISC: discrimination of mitochondrial targeting seq

| | | | |
|---|---|---|---|
| R content: | 0 | Hyd Moment(75): | 5.33 |
| Hyd Moment(95): | 5.59 | G content: | 3 |
| D/E content: | 2 | S/T content: | 4 |
| Score: −7.44 | | | |

| | |
|---|---|
| Gavel: | prediction of cleavage sites for mitochondrial preseq |
| | cleavage site motif not found |

NUCDISC: discrimination of nuclear localization signals

pat4: none
pat7: none
bipartite: none
content of basic residues: 11.4%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

| | Prediction: cytoplasmic |
|---|---|
| | Reliability: 89 |
| COIL: | Lupas's algorithm to detect coiled-coil regions |
| | total: 0 residues |

Final Results (k = 9/23):

52.2%: cytoplasmic
34.8%: nuclear
13.0%: mitochondrial

>> prediction for CG55778-03 is cyt (k = 23)

A search of the NOV45a protein against the Geneseq database, a proprietary database that contains sequences published in patents and patent publication, yielded several homologous proteins shown in Table 45D.

TABLE 45D
Geneseq Results for NOV45a

| | | NOV45a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP53592 | Human NOV16c protein SEQ | 1 . . . 210 | 210/210 (100%) | e−124 |
| | ID NO: 48 - Homo sapiens, 210 | 1 . . . 210 | 210/210 (100%) | |
| | aa. [WO200262999-A2, 15- | | | |
| | AUG-2002] | | | |
| ABP53591 | Human NOV16b protein SEQ | 1 . . . 210 | 208/251 (82%) | e−117 |
| | ID NO: 46 - Homo sapiens, 251 | 1 . . . 251 | 209/251 (82%) | |
| | aa. [WO200262999-A2, 15- | | | |
| | AUG-2002] | | | |
| AAM79279 | Human protein SEQ ID NO | 1 . . . 210 | 205/251 (81%) | e−114 |
| | 1941 - Homo sapiens, 263 aa. | 1 . . . 250 | 207/251 (81%) | |
| | [WO200157190-A2, 09-AUG- | | | |
| | 2001] | | | |
| ABU11795 | Human MDDT polypeptide SEQ | 1 . . . 156 | 152/156 (97%) | 5e−89 |
| | ID 742 - Homo sapiens, 215 aa. | 31 . . . 185 | 154/156 (98%) | |
| | [WO200279449-A2, 10-OCT- | | | |
| | 2002] | | | |
| AAM80263 | Human protein SEQ ID NO | 1 . . . 156 | 151/156 (96%) | 3e−88 |
| | 3909 - Homo sapiens, 264 aa. | 11 . . . 165 | 152/156 (96%) | |
| | [WO200157190-A2, 09-AUG- | | | |
| | 2001] | | | |

In a BLAST search of public sequence databases, the NOV45a protein was found to have homology to the proteins shown in the BLASTP data in Table 45E.

TABLE 45E
Public BLASTP Results for NOV45a

| | | NOV45a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAC54567 | Aldo-keto reductase related | 1 . . . 210 | 207/210 (98%) | e−121 |
| | protein 3 - Homo sapiens | 1 . . . 209 | 209/210 (98%) | |
| | (Human), 222 aa. | | | |
| BAC54565 | Aldo-keto reductase related | 1 . . . 210 | 208/251 (82%) | e−115 |
| | protein 1 - Homo sapiens | 1 . . . 250 | 209/251 (82%) | |
| | (Human), 250 aa. | | | |
| BAC54566 | Aldo-keto reductase related | 1 . . . 210 | 207/251 (82%) | e−115 |
| | protein 2 - Homo sapiens | 1 . . . 250 | 209/251 (82%) | |
| | (Human), 263 aa. | | | |
| Q9BU71 | Similar to aldo-keto reductase - | 1 . . . 156 | 153/156 (98%) | 6e−89 |
| | Homo sapiens (Human), 307 aa. | 1 . . . 155 | 154/156 (98%) | |
| Q96JD6 | Aldo-keto reductase loopADR - | 1 . . . 156 | 153/156 (98%) | 6e−89 |
| | Homo sapiens (Human), 320 aa. | 1 . . . 155 | 154/156 (98%) | |

PFam analysis indicates that the NOV45a protein contains the domains shown in the Table 45F.

TABLE 45F
Domain Analysis of NOV45a

| | NOV45a | Identities/Similarities | Expect |
|---|---|---|---|
| Pfam Domain | Match Region | for the Matched Region | Value |
| aldo_ket_red | 2 . . . 203 | 87/369 (24%) | 8.2e−37 |
| | | 157/369 (43%) | |

### Example 46

The NOV46 clone was analyzed, and the nucleotide and encoded polypeptide sequences are shown in Table 46A.

TABLE 46A

NOV46 Sequence Analysis

| | | |
|---|---|---|
| NOV46a, CG55794-03 | SEQ ID NO: 699 | 1222 bp |
| DNA Sequence | ORF Start: ATG at 41 | ORF Stop: TAA at 1163 |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGCTGGGATATGATAGATCCTTAGCCCAACACAGACAAGAGATT
GTGGACAAGTCAGTGAGTCCATGGAGCCTGGAGACGTATTCCTATAACATATACCACCCCATGGGAGA
GATCTATGAGTGGATGAGAGAGATCAGTGAGAAGTACAAGGAAGTGGTGACACAGCAATTCCTAGGAG
TGACCTATGAGACCCACCCCATATATTATCTGAAGATCAGCCAACCATCTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAGTATTGGTGCCTCTAGAAACTGCCAAGATCAAACATTCTGTGGGACAGGGCCAGTGTCTGAACCAG
AGACTAAAGCTGTTGCCAGCTTCATAGAGAGCAAGAAGGATGATATTTTGTGCTTCCTGACCATGCAC
TCTTATGGGCAGTTAATTCTCACACCTTACGGCTACACCAAAAATAAATCAAGTAACCACCCAGAAAT
GATTCAAGTTGGACAGAAGGCAGCAAATGCATTGAAAGCAAAGTATGGAACCAATTATAGAGTTGGAT
CGAGTGCAGATATTTTATATGCCTCATCAGGGTCTTCAAGAGATTGGGCCCGAGACATTGGGATTCCC
TTCTCATATACGTTTGAGCTGAGGGACAGTGGAACATATGGGTTTGTTCTGCCAGAAGCTCAGATCCA
GCCCACCTGTGAGGAGACCATGGAGGCTGTGCTGTCAGTCCTGGATGATGTGTATGCGAAACACTGGC
ACTCGGACAGTGCTGGAAGGGTGACATCTGCCACTATGCTGCTGGGCCTGCTGGTGTCCTGCATGTCT
CTTCTCTAAGTGCATTCTGCCCAGGCCTGCTCAACCCCAGTGGCATGAGTGTGGCTTGGAGGAACG

| | | | |
|---|---|---|---|
| NOV46a, CG55794-03 | | | |
| Protein Sequence | SEQ ID NO: 700 | 1374 aa | MW at 42580.0kD |

MKPLLETLYLLGMLVPGGLGYDRSLAQHRQEIVDKSVSPWSLETYSYNIYHPMGEIYEWNREISEKYK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPVSEPETKAVASFIESKKDDILCFLTMHSYGQLILTPYGYTKNKSSNHPEMIQVGQKAANALKA
KYGTNYRVGSSADILYASSGSSRDWARDIGIPFSYTFELRLSGTYGFVLPEAQIQPTCEETMEAVLSV
LDDVYAKHWHSDSAGRVTSATMLLGLLVSCMSLL

| | | |
|---|---|---|
| NOV46b, 210223559 | SEQ ID NO: 701 | 1579 bp |
| DNA Sequence | ORF Start: at 67 | ORf Stop: TAA at 1396 |

CCAATTGACAAGCTTTTGATTTTAACGACTTTTAACGACAACTTGAGAAGATCAAAAAACAACTAATT
ATTCGAAACGATGAGATTTCCTTCAATTTTTACTGCTGTTTTATTCGCAGCATCCTCCGCATTAGCTG
CTCCAGTCAACACTACAACAGAAGATGAAACGGCACAAATTCCGGCTGAAGCTGTCATCGGTTACTCA
GATTTAGAAGGGGATTTCGATGTTGCTGTTTTGCCATTTTCCAACAGCACAAATAACGGGTTATTGTT
TATAAATACTACTATTGCCAGCATTGCTGCTAAAGAAGAAGGGGTATCTCTCGAGAAAAGATATGATA
GATCCTTAGCCCAACACAGACAAGAGATTGTGGACAAGTCAGTGAGTCCATGGAGCCTGGAGACGTAT
TCCTATAACATATACCACCCCATGGGAGAGATCTATGAGTGGATGAGAGAGATCAGTGAGAAGTACAA
GGAAGTGGTGACACAGCATTTCCTAGGAGTGACCTATGAGACCCACCCCATGTATTATCTGAAGATCA
GCCAACCATCTGGTAATCCCAAGAAAATCATTTGGATGGACTGTGGAATTCACGCCAGAGAATGGATT
GCTCCTGCTTTTTGCCAATGGTTCGTCAAAGAAATTCTACAAAACCATAAAGACAACTCAAGTATACG
CAAGCTTCTTAGGAACCTGGACTTCTATGTCCTTCCAGTTCTTAACATAGATGGTTATATCTACACTT
GGACAACTGATCGTCTTTGGAGGAAATCCCGTTCACCCCATAATAATGGCACATGTTTTGGGACGGAT
CTCAATCGAAATTTCAATGCATCTTGGTGTAGTATTGGTGCCTCTAGAAACTGCCAAGATCAAACATT
CTGTGGGACAGGGCCAGTGTCTGAACCAGAGACTAAAGCTGTTGCCAGCTTCATAGAGAGCAAGAAGG
ATGATATTTTGTGCTTCCTGACCATGCACTCTTATGGGCAGTTAATTCTCACACCTTACGGCTACACC
AAAAATAAATCAAGTAACCACCCAGAAATGATTCAAGTTGGACAGAAGGCAGCAAATGCATTGAAAGC
AAAGTATGGAACCAATTATAGAGTTGGATCGAGTGCAGATATTTTATATGCcTCATCAGGGTCTTCAA
GAGATTGGGCCCGAGACATTGGGATTCCCTTCTCATATACGTTTGAGCTGAGGGACAGTGGAACGTAT
GGGTTTGTTCTGCCAGAAGCTCAGATCCAGCCCACCTGTGAGGAGACCATGGAGGCTGTGCTGTCAGT
CCTGGATGATGTGTATGCGAAACACTGGCACTCGGACAGTGCTGGAAGGGTGACATCTGCCACTATGC
TGCTGGGCCTGCTGGTGTCCTGCATGTCTCTTCTCTAAGCGGCCGCCAGCTTTCTAGAACAAAAACTC
ATCTCAGAAGAGGATCTGAATAGCGCCGTCGACCATCATCATCATCATCATTGAGTTTGTAGCCTTAG
ACATGACTGTTCCTCAGTTCAAGTTGGGCACTTACGAGAAGACCGGTCTTGCTAGATTCTAATCAAGA
GGATGTCAGAATGCC

| | | | |
|---|---|---|---|
| NOV46b, 210223559 | | | |
| Protein Sequence | SEQ ID NO: 702 | 443 aa | MW at 49842.7kD |

LFETMRFPSIFTAVLFAASSALAAPVNTTTEDETAQIPAEAVIGYSDLEGDFDVAVLPFSNSTNNGLL
FINTTIASIAAKEEGVSLEKRYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGPFSYTFELRDSGTYGFVLPEAQIQPTCEAETMEAVLS
VLDDVYAKHWHSDSAGRVTSATMLLGLLVSCMSLL

| | | |
|---|---|---|
| NOV46c, 210223626 | SEQ ID NO: 703 | 1607 bp |
| DNA Sequence | ORF Start: at 74 | ORF Stop: TAA at 1421 |

ACTGGTTCCAATTGACAAGCTTTTGATTTTAACGACTTTTAACGACAACTTGAGAAGATCAAAAAACA
ACTAATTATTCGAAACGATGAGATTTCCTTCAATTTTTACTGCTGTTTTATTCGCAGCATCCTCCGCA
TTAGCTGCTCCAGTCAACACTACAACAGAAGATGAAACGGCACAAATTCCGGCTGAAGCTGTCATCGG
TTACTCAGATTTAGAAGGGGATTTCGATGTTGCTGTTTTGCCATTTTCCAACAGCACAAATAACGGGT
TATTGTTTATAAATACTACTATTGCCAGCATTGCTGCTAAAGAAGAAGGGGTATCTCTCGAGAAAAGA
TATGATAGATCCTTAGCCCAACACAGACAAGAGATTGTGGACAAGTCAGTGAGTCCATGGAGCCTGGA
GACGTATTCCTATAACATATACCACCCCATGGGAGAGATCTATGAGTGGATGAGAGAGATCAGTGAGA
AGTACAAGGAAGTGGTGACACAGCATTTCCTAGGAGTGACCTATGAGACCAACCCCATGTATTATCTG
AAGATCAGCCAACCATCTGGTAATCCCAAGAAAATCATTTGGATGGACTGTGGAATTCACGCCAGAGA
ATGGATTGCTCCTGCTTTTTGCCAATGGTTCGTCAAAGAAATTCTACAAAACCATAAAGACAACTCAA
GTATACGCAAGCTTCTTAGGAACCTGGACTTCTATGTCCTTCCAGTTCTTAACATAGATGGTTATATC
TACACTTGGACAACTGATCGTCTTTGGAGGAAATCCCGTTCACCCCATAATAATGGCACATGTTTTGG
GACGGATCTCAATCGAAATTTCAATGCATCTTGGTGTAGTATTGGTGCCTCTAGAAACTGCCAAGATC
AAACATTCTGTGGGACAGGGCCAGTGTCTGAACCAGAGACTAAAGCTGTTGCCAGCTTCATAACAGGG
CTACACCAAAAATAAATCAAGTAACCACCCAGAAATGATTCAAGTTGGACAGAAGGCAGCAAATGCAT
TGAAAGCAAAGTATGGAACCAATTATAGAGTTGGATCGAGTGCAGATATTTTATATGCCTCATCAGGG
TCTTCAAGAGATTGGGCCCGAGACATTGGGATTCCCTTCTCATATACGTTTGAGCTGAGGGACAGTGG
TCTTCAAGAGATTGGGCCCGAGACATTGGGATTCCCTTCTCATATACGTTTGAGCTGAGGGAAAGTGG
AACGTATGGGTTTGTTCTGCCAGAAGCTCAGATCCAGCCCACCTGTGAGGAGACCATGGAGGCTGTGC
TGTCAGTCCTGGATGATGTGTATGCGAAACACTGGCACTCGGACAGTGCTGGAAGGGTGACATCTGCC
ACTATGCTGCTGGGCCTGCTGGTGTCCTGCATGTCTCTTCTCCACCATCACCACCATCACTAAGCGGC
CGCCAGCTTTCTAGAACAAAAACTCATCTCAGAAGAGGATCTGAATAGCGCCGTCGACCATCATCATC
ATCATCATTGAGTTTGTAGCCTTAGACATGACTGTTCCTCAGTTCAAGTTGGGCACTTACGAGAAGAC
CGGTCTTGCTAGCATTCTAATCAAGAGGATGTCAGAATGCCATT

| | | | |
|---|---|---|---|
| NOV46c, 210223626 | | | |
| Protein Sequence | SEQ ID NO: 704 | 449 aa | MW at 50665.6kD |

LFETMRFPSIFTAVLFAASSALAAPVNTTTEDETAQIPAEAVIGYSDLEGDFDVAVLPFSNSTNNGLL
FINTTIASIAAKEEGVSLEKRYDRSLAQHRQEIVDKSVSPWSLETYSYNIYHPMGEIYENMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLLHHHHHH

| | | |
|---|---|---|
| NOV46d, 171095097 | SEQ ID NO: 705 | 1148 bp |
| DNA Sequence | ORF Start: at 2 | ORF Stop: TAA at 1127 |

CACCATGAAGCCTCTGCTTGAAACCCTTTATCTTTTGGGGATGCTGGTTCCTGGAGGGCTGGGATATG
ATAGATCCTTAGCCCAACACAGACAAGAGATTGTGGACAAGTCAGTGAGTCCATGGAGCCTGGAGACG
TATTCCTATAACATATACCACCCCATGGGAGAGATCTATGAGTGGATGAGAGAGATCAGTGAGAAGTA
CAAGGAAGTGGTGACACAGCATTTCCTAGGAGTGACCTATGAGACCCACCCCATGTATTATCTGAAGA
TCAGCCACCATCTGGTAATCCCAAGAAAATCATTTGGATGGACTGTGGAATTCACGCCAGAAGAATGG
ATTGCTCCTGCTTTTTGCCAATGGTTCGTCAAAGAAATTCTACAAAACCATAAAGACAACTCAAGTAT
ACGCAAGCTTCTTAGGAACCTGGACTTCTATGTCCTTCCAGTTCTTAACATAGATGGTTATATCTACA
CTTGGACAACTGATCGTCTTTGGAGGAAATCCCGTTCACCCCATAATAATGGCACATGTTTTGGGACG
GATCTCAATCGAAATTTCAATGCATCTTGGTGTAGTATTGGTGCCTCTAGAAACTGCCAAGATCAAAC
ATTCTGTGGGACAGGGCCAGTGTCTGAACCAGAGACTAAAGCTGTTGCCAGCTTCATAGAGAGCAAGA
AGGATGATATTTTGTGCTTCCTGACCATGCACTCTTATGGGCAGTTAATTCTCACACCTTACGGCTAC
ACCAAAAATAAATCAAGTAACCACCCAGAAATGATTCAAGTTGGACAGAAGGCAGCAAATGCATTGAA
AGCAAAGTATGGAACCAATTATAGAGTTGGATCGAGTGCAGATATTTTATATGCCTCATCAGGGTCTT
CAAGAGATTGGGCCCGAGACATTGGGATTCCCTTCTCATATACGTTTGAGCTGAGGGACAGTGGAACG
TATGGGTTTGTTCTGCCAGAAGCTCAGATCCAGCCCACCTGTGAGGAGACCATGGAGGCTGTGCTGTC
AGTCCTGGATGATGTGTATGCGAAACACTGGCACTCGGACAGTGCTGGAAGGGTGACATCTGCCACTA
TGCTGCTGGGCCTGCTGGTGTCCTGCATGTCTCTTCTCTAAGCGGCCGCTCGAGTCTAGA

| | | | |
|---|---|---|---|
| NOV46d, 171095097 | | | |
| Protein Sequence | SEQ ID NO: 706 | 375 aa | MW at 42630.1kD |

TMKPLLETLYLLGMLVPGGLGYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLL

| | | |
|---|---|---|
| NOV46e, 183852229 | SEQ ID NO: 707 | 1148 bp |
| DNA Sequence | ORF Start: at 3 | ORF Stop: TAA at 1146 |

CCACCATGAAGCCTCTGCTTGAAACCCTTTATCTTTTGGGGATGCTGGTTCCTGGAGGGCTGGGATAT
GATAGATCCTTAGCCCAACACAGACAAGAGATTGTGGACAAGTCAGTGAGTCCATGGAGCCTGGAGAC
GTATTCCTATAACATATACCACCCCATGGGAGAGATCTATGAGTGGATGAGAGAGATCAGTGAGAAGT
ACAAGGAAGTGGTGACACAGCATTTCCTAGGAGTGACCTATGAGACCCACCCCATGTATTATCTGAAG
ATCAGCCAACCATCTGGTAATCCCAAGAAAATCATTTGGATGGACTGTGGAATTCACGCCAGAGAATG
GATTGCTCCTGCTTTTTGCCAATGGTTCGTCAAAGAAATTCTACAAAACCATAAAGACAACTCAAGTA
TACGCAAGCTTCTTAGGAACCTGGACTTCTATGTCCTTCCAGTTCTTAACATAGATGGTTATATCTAC
ACTTGGACAACTGATCGTCTTTGGAGGAAATCCCGTTCACCCCATAATAATGGCACATGTTTTGGGAC
GGATCTCAATCGAAATTTCAATGCATCTIGGTGTAGTATTGGTGCCTCTAGAAACTGCCAAGATCAAA
CATTCTGTGGGACAGGGCCAGTGTCTGAACCAGAGACTAAAGCTGTTGCCAGCTTCATAGAGAGCAAG
AAGGATGATATTTTGTGCTTCCTGACCATGCACTCTTATGGGCAGTTAATTCTCACACCTTACGGCTA
CACCAAAAATAAATCAAGTAACCACCCAGAAATGATTCAAGTTGGACAGAAGGCAGCAAATGCATTGA
AAGCAAAGTATGGAACCAATTATAGAGTTGGATCGAGTGCAGATATTTTATATGCCTCATCAGGGTCT
TCAAGAGATTGGGCCCGAGACATTGGGATTCCCTTCTCATATACGTTTGAGCTGAGGGACAGTGGAAC
GTATGGGTTTGTTCTGCCAGAAGCTCAGATCCAGCCCACCTGTGAGGAGACCATGGAGGCTGTGCTGT
CAGTCCTGGATGATGTGTATGCGAAACACTGGCACTCGGACAGTGCTGGAAGGGTGACATCTGCCACT
ATGCTGCTGGGCCTGCTGGTGTCCTGCATGTCTCTTCTCCACCATCACCACCATCACTAA

| | | | |
|---|---|---|---|
| NOV46e, 183852229 | | | |
| Protein Sequence | SEQ ID NO: 708 | 381 aa | MW at 43452.9kD |

TMKPLLETLYLLGMLVPGGLGYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCOWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSnHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLLHHHHHH

| | | |
|---|---|---|
| NOV46f, 183852264 | SEQ ID NO: 709 | 1151 bp |
| DNA Sequence | ORF Start: at 3 | ORF Stop: TAA at 1149 |

CCACCATGGGCCACCATCACCACCATCACAAGCCTCTGCTTGAAACCCTTTATCTTTTGGGGATGCTG
GTTCCTGGAGGGCTGGGATATGATAGATCCTTAGCCCAACACAGACAAGAGATTGTGGACAAGTCAGT
GAGTCCATGGAGCCTGGAGACGTATTCCTATAACATATACCACCCCATGGGAGAGATCTATGAGTGGA
TGAGAGAGATCAGTGAGAAGTACAAGGAAGTGGTGACACAGCATTTCCTAGGAGTGACCTATGAGACC
CACCCCATGTATTATCTGAAGATCAGCCAACCATCTGGTAATCCCAAGAAAATCATTTGGATGGACTG
TGGAATTCACGCCAGAGAATGGATTGCTCCTGCTTTTTGCCAATGGTTCGTCAAAGAAATTCTACAAA
ACCATAAAGACAACTCAAGTATACGCAAGCTTCTTAGGAACCTGGACTTCTATGTCCTTCCAGTTCTT
AACATAGATGGTTATATCTACACTTGGACAACTGATCGTCTTTGGAGGAAATCCCGTTCACCCCATAA
TAATGGCACATGTTTTGGGACGGATCTCAATCGAAATTTCAATGCATCTTGGTGTAGTATTGGTGCCT
CTAGAAACTGCCAAGATCAAACATTCTGTGGGACAGGGCCAGTGTCTGAACCAGAGACTAAAGCTGTT
GCCAGCTTCATAGAGAGCAAGAAGGATGATATTTTGTGCTTCCTGACCATGCACTCTTATGGGCAGTT
AATTCTCACACCTTACGGCTACACCAAAAATAAATCAAGTAACCACCCAGAAATGATTCAAGTTGGAC
AGAAGGCAGCAAATGCATTGAAAGCAAAGTATGGAACCAATTATAGAGTTGGATCGAGTGCAGATATT
TTATATGCCTCATCAGGGTCTTCAAGAGATTGGGCCCGAGACATTGGGATTCCCTTCTCATATACGTT
TGAGCTGAGGGACAGTGGAACGTATGGGTTTGTTCTGCCAGAAGCTCAGATCCAGCCCACCTGTGAGG
AGACCATGGAGGCTGTGCTGTCAGTCCTGGATGATGTGTATGCGAAACACTGGCACTCGGACAGTGCT
GGAAGGGTGACATCTGCCACTATGCTGCTGGGCCTGCTGGTGTCCTGCATGTCTCTTCTCTAA

| | | | |
|---|---|---|---|
| NOV46f, 183852264 | | | |
| Protein Sequence | SEQ ID NO: 710 | 382 aa | MW at 43510.0kD |

TMGHHHHHHKPLLETLYLLGMLVPGGLGYDRSLAQHRQEIVDKSVSPWSLETYSYNIYHPMGEIYEWM
REISEKYKEVVTQHFLGVTYETHPMYYLKISQPSGNPKKIIWMDCGIHAREWIAPAFCQWFVKEILQN
HKDNSSIRKLLRNLDFYVLPVLNIDGYIYTWTTDRLWRKSRSPHNNGTCFGTDLNRNFNASWCSIGAS
RNCQDQTFCGTGPVSEPETKAVASFIESKKDDILCFLTMHSYGQLILTPYGYTKNKSSNHPEMIQVGQ
KAANALKAKYGTNYRVGSSANILYASSGSSRNWARDIGIPFSYTFELRDSGTYGFVLPEAQIQPTCEE
TMEAVLSVLDDVYAKHWHSDSAGRVTSATMLLGLLVSCMSLL

| | | |
|---|---|---|
| NOV46g, 183852410 | SEQ ID NO: 711 | 1162 bp |
| DNA Sequence | ORF Start: at 2 | ORF Stop: TAA at 1148 |

CACCATGGGCCACCATCACCACCATCACAAGCCTCTGCTTGAAACCCTTTATCTTTTGGGGATGCTGG
TTCCTGGAGGGCTGGGATATGATAGATCCTTAGCCCAACACAGACAAGAGATTGTGGACAAGTCAGTG
AGTCCATGGAGCCTGGAGACGTATTCCTATAACATATACCACCCCATGGGAGAGATCTATGAGTGGAT
GAGAGAGATCAGTGAGAAGTACAAGGAAGTGGTGACACAGCATTTCCTAGGAGTGACCTATGAGACCC
ACCCCATGTATTATCTGAAGATCAGCCAACCATCTGGTAATCCCAAGAAAATCATTTGGATGGACTGT
GGAATTCACGCCAGAGAATGGATTGCTCCTGCTTTTTGCCAATGGTTCGTCAAAGAAATTCTACAAAA
CCATAAAGACAACTCAAGTATACGCAAGCTTCTTAGGAACCTGGACTTCTATGTCCTTCCAGTTCTTA
ACATAGATGGTTATATCTACACTTGGACAACTGATCGTCTTTGGAGGAAATCCCGTTCACCCCATAAT
AATGGCACATGTTTTGGGACGGATCTCAATCGAAATTTCAATGCATCTTGGTGTAGTATTGGTGCCTC
TAGAAACTGCCAAGATCAAACATTCTGTGGGACAGGGCCAGTGTCTGAACCAGAGACTAAAGCTGTTG
CCAGCTTCATAGAGAGCAAGAAGGATGATATTTTGTGCTTCCTGACCATGCACTCTTATGGGCAGTTA
ATTCTCACACCTTACGGCTACACCAAAAATAAATCAAGTAACCACCCAGAAATGATTCAAGTTGGACA
GAAGGCAGCAAATGCATTGAAAGCAAAGTATGGAACCAATTATAGAGTTGGATCGAGTGCAGATATTT
TATATGCCTCATCAGGGTCTTCAAGAGATTGGGCCCGAGACATTGGGATTCCCTTCTCATATACGTTT
GAGCTGAGGGACAGTGGAACGTATGGGTTTGTTCTGCCAGAAGCTCAGATCCAGCCCACCTGTGAGGA
GACCATGGAGGCTGTGCTGTCAGTCCTGGATGATGTGTATGCGAAACACTGGCACTCGGACAGTGCTG
GAAGGGTGACATCTGCCACTATGCTGCTGGGCCTGCTGGTGTCCTGCATGTCTCTTCTCTAAGCGGCC
GCTTTC

| | | | |
|---|---|---|---|
| NOV46g, 183852410 | | | |
| Protein Sequence | SEQ ID NO: 712 | 382 aa | MW at 43510.0kD |

TMGHHHHHHKPLLETLYLLGMLVPGGLGYDRSLAQHRQEEVDKSVSPWSLETYSYNIYHPMGEIYEWN
REISEKYKEVVTQHFLGVTYETHPMYYLKISQPSGNPKKIIWMDCGIHAREWIAPAFCQWFVKEILQN
HKDNSSIRKLLRNLDFYVLPVLNIDGYIYTWTTDRLWRKSRSPHNNGTCFGTDLNRNFNASWCSIGAS
RNCQDQTFCGTGPVSEPETKAVASFIESKKDDILCFLTMHSYGQLILTPYGYTKNKSSNHPEMIQVGQ
KAANALKAKYGTNYRVGSSADILYASSGSSRDWARDIGIPFSYTFELRDSGTYGFVLPEAQIQPTCEE
TMEAVLSVLDDVYAKHWHSDSAGRVTSATMLLGLLVSCMSLL

| | | |
|---|---|---|
| NOV46h, 183523337 | SEQ ID NO: 713 | 1162 bp |
| DNA Sequence | ORF Start: at 2 | ORF Stop: TAA at 1148 |

CACCATGGGCCACCATCACCACCATCACAAGCCTCTGCTTGAAACCCTTTATCTTTTGGGGATGCTGG
TTCCTGGAGGGCTGGGATATGATAGATCCTTAGCCCAACACAGACAAGAGATTGTGGACAAGTCAGTG
AGTCCATGGAGCCTGGAGACGTATTCCTATAACATATACCACCCCATGGGAGAGATCTATGAGTGGAT
GAGAGAGATCAGTGAGAAGTACAAGGAAGTGGTGACACAGCATTTCCTAGGAGTGACCTATGAGACCC
ACCCCATGTATTATCTGAAGATCAGCCAACCATCTGGTAATCCCAAGAAAATCATTTGGATGGACTGT
GGAATTCACGCCAGAGAATGGATTGCTCCTGCTTTTTGCCAATGGTTCGTCAAAGAAATTCTACAAAA
CCATAAAGACAACTCAAGTATACGCAAGCTTCTTAGGAACCTGGACTTCTATGTCCTTCCAGTTCTTA
ACATAGATGGTTATATCTACACTTGGACAACTGATCGTCTTTGGAGGAAATCCCGTTCACCCCATAAT
AATGGCACATGTTTTGGGACGGATCTCAATCGAAATTTCAATGCATCTTGGTGTAGTATTGGTGCCTC
TAGAAACTGCCAAGATCAAACATTCTGTGGGACAGGGCCAGTGTCTGAACCAGAGACTAAAGCTGTTG
CCAGCTTCATAGAGAGCAAGAAGGATGATATTTTGTGCTTCCTGACCATGCACTCTTATGGGCAGTTA
ATTCTCACACCTTACGGCTACACCAAAAATAAATCAAGTAACCACCCAGAAATGATTCAAGTTGGACA
GAAGGCAGCAAATGCATTGAAAGCAAAGTATGGAACCAATTATAGAGTTGGATCGAGTGCAGATATTT
TATATGCCTCATCAGGGTCTTCAAGAGATTGGGCCCGAGACATTGGGATTCCCTTCTCATATACGTTT
GAGCTGAGGGACAGTGGAACGTATGGGTTTGTTCTGCCAGAAGCTCAGATCCAGCCCACCTGTGAGGA
GACCATGGAGGCTGTGCTGTCAGTCCTGGATGATGTGTATGCGAAACACTGGCACTCGGACAGTGCTG
GAAGGGTGACATCTGCCACTATGCTGCTGGGCCTGCTGGTGTCCTGCATGTCTCTTCTCTAAGCGGCC
GCTTTC

| | | | |
|---|---|---|---|
| NOV46h, 183523337 | | | |
| Protein Sequence | SEQ ID NO: 714 | 382 aa | MW at 43510.0kD |

TMGHHHHHHKPLLETLYLLGMLVPGGLGYDRSLAQHRQEIVDKSVSPWSLETYSYNIYHPMGEIYEWM
REISEKYKEVVTQHFLGVTYETHPMYYLKISQPSGNPKKIIWMDCGIRAREWIAPAFCQWFVKEILQN
HKDNSSIRKLLRNLDFYVLPVLNIDGYIYTWTTDRLWRKSRSPHNNGTCFGTDLNRNFNASWCSIGAS
RNCQDQTFCGTGPVSEPETKAVASFIESKKDDILCFLTMHSYGQLILTPYGYTKNKSSNHPEMIQVGQ
KAANALKAKYGTNYRVGSSADILYASSGSSRDWARDIGIPFSYTFELRDSGTYGFVLPEAQIQPTCEE
TMEAVLSVLDDVYAKHWHSDSAGRVTSATMLLGLLVSCMSLL

| | | |
|---|---|---|
| NOV46i, CG55794-01 | SEQ ID NO: 715 | 1196 bp |
| DNA Sequence | ORF Start: ATG at 16 | ORF Stop: TAA at 1138 |

TTACTGTGTGGCAGAATGAAGCCTCTGCTTGAAACCCTTTATCTTTTGGGGATGCTGGTTCCTGGAGG
GCTGGGATATGATAGATCCTTAGCCCAACACAGACAAGAGATTGTGGACAAGTCAGTGAGTCCATGGA
GCCTGGAGACGTATTCCTATAACATATACCACCCCATGGGAGAGATCAATGAGTGGATGAGAGAGATC
AGTGAGAAGTACAAGGAAGTGGTGACACAGCATTTCCTAGGAGTGACCTATGAGACCCACCCCATATA
TTATCTGAAGATCAGCCAACCATCTGGTAATCCCAAGAAAATCATTTGGATGGGCTGTGGAATTCACG
GTTTTGGGACGGATCTCAATCGAAATTTCAATGCATCTTGGTGTAGTATTGGTGCCTCTAGAAACTGC
CAAGATCAAACATTCTGTGGGACAGGGCCAGTGTCTGAACCAGAGACTAAAGCTGTTGCCAGCTTCAT
AGAGAGCAAGAAGGATGATATTTTGTGCTTCCTGACCATGCACTCTTATGGGCAGTTAATTCTCACAC
GTTTTGGGACGGATCTCAATCGAAATTTCAATGCATCTTGGTGTAGTATTGGTGCCTCTAGAAACTGC
CAAGATCAAACATTCTGTGGGACAGGGCCAGTGTCTGAACCAGAGACTAAAGCTGTTGCCAGCTTCAT
AGAGAGCAAGAAGGATGATATTTTGTGCTTCCTGACCATGCACTCTTATGGGGCAGTTAATTCTCACA
CTTACGGCTACACCAAAAATAAATCAAGTAACCACCCAGAAATGATTCAAGTTGGACAGAAGGCAGCA
AATGCATTGAAAGCAAAGTATGGAACCAATTATAGAGTTGGATCGAGTGCAGATATTTTATATGCCTC
ATCAGGGTCTTCAAGAGATTGGGCCCGAGACATTGGGATTCCCTTCTCATATACGTTTGAGCTGAGGG
ACAGTGGAACATATGGGTTTGTTCTGCCAGAAGCTCAGATCCAGCCCACCTGTGAGGAGACCATGGAG
GCTGTGCTGTCAGTCCTGGATGATGTGTATGCGAAACACTGGCACTCGGACAGTGCTGGAAGGGTGAC
ATCTGCCACTATGCTGCTGGGCCTGCTGGTGTCCTGCATGTCTCTTCTCTAAGTGCATCCTGCCCAGG
CCTGCTCAACCCCAGTGGCATGAGTGTGGCTGGAGGAACG

| | | | |
|---|---|---|---|
| NOV46i, GG55794-01 | | | |
| Protein Sequence | SEQ ID NO: 716 | 374 aa | MW at 42472.9kD |

MKPLLETLYLLGMLVPGGLGYDRSLAQHRQEIVDKSVSPWSLETYSYNIYHPMGEINEWMREISEKYK
EVVTQHFLGVTYETHPIYYLKISQPSGNPKKIIWMGCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPVSEPETKAVASFIESKKDDILCFLTMHSYGQLILTPYGYTKNKSSNHPEMIQVGQKAANLAKA
KYGTNYRVGSSADILYASSGSSRDWARDIGIPFSYTFELRDSGTYGFVLPEAQIQPTCEETMEAVLSV
LDDVYAKHWHSDSAGRVTSATMLLGLLVSCMSLL

| | | |
|---|---|---|
| NOV46j, CG55794-02 | SEQ ID NO: 717 | 1008 bp |
| DNA Sequence | ORF Start: at 1 | ORF Stop: end of sequence |

TATGATAGATCCTTAGCCCAACACAGACAAGAGATTGTGGACAAGTCAGTGAGTCCATGGAGCCTGGA
GACGTATTCCTATAACATATACCACCCCATGGGAGAGATCTATGAGTGGATGAGAGAGATCAGTGAGA
AGTACAAGGAAGTGGTGACACAGCATTTCCTAGGAGTGACCTATGAGACCCACCCCATGTATTATCTG
AAGATCAGCCAACCATCTGGTAATCCCAAGAAAATCATTTGGATGGACTGTGGAATTCACGCCAGAGA
ATGGATTGCTCCTGCTTTTTGCCAATGGTTCGTCAAAGAAATTCTACAAAACCATAAAGACAACTCAA
GTATACGCAAGCTCCTTAGGAACCTGGACTTCTATGTCCTTCCAGTTCTTAACATAGATGGTTATATC
TACACTTGGACAACTGATCGTCTTTGGAGGAAATCCCGTTCACCCCATAATAATGGCACATGTTTTGG
GACGGATCTCAATCGAAATTTCAATGCATCTTGGTGTAGTATTGGTGCCTCTAGAAACTGCCAAGATC
AAACATTCTGTGGGACAGGGCCAGTGTCTGAACCAGAGACTAAAGCTGTTGCCAGCTTCATAGAGAGC
AAGAAGGATGATATTTTGTGCTTCCTGACCATGCACTCTTATGGGCAGTTAATTCTCACACCTTACGG
CTACACCAAAAATAAATCAAGTAACCACCCAGAAATGATTCAAGTTGGACAGAAGGCAGCAAATGCAT
TGAAAGCAAAGTATGGAACCAATTATAGAGTTGGATCGAGTGCAGATATTTTATATGCCTCATCAGGG
TCTTCAAGAGATTGGGCCCGAGACATTGGGATTCCCTTCTCATATACGTTTGAGCTGAGGGACAGTGG
AACATATGGGTTTGTTCTGCCAGAAGCTCAGATCCAGCCCACCTGTGAGGAGACCATGGAGGCTGTGC
TGTCAGTCCTGGATGATGTGTATGCGAAACACTGGCACTCGGACAGTGCTGGAAGG

| | | | |
|---|---|---|---|
| NOV46j, CG55794-02 | | | |
| Protein Sequence | SEQ ID NO: 718 | 336 aa | MW at 38597.1kD |

YDRSLAQHROEIVDKSVSPWSLETYSYNIYHPMGEIYEWMREISEKYKBVVTQHFLGVTYETHPMYYL
KISQPSGNPKKIIWMDCGIHAREWIAPAFCQWFVKEILQNHKDNSSIRKLLRNLDFYVLPVLNIDGYI
YTWTTDRLWRKSRSPHNNGTCFGTDLNRNFNASWCSIGASRNCQDQTFCGTGPVSEPETKAVASFIES
KKDDILCFLTMHSYGQLILTPYGYTKNKSSNHPEMIQVGQKAANALKAKYGTNYRVGSSADILYASSG
SSRDWARDIGIPFSYTFELRDSGTYGFTLPEAQIQPTCEETMEAVLSVLDDVYAKHWHSDSAGR

| | | |
|---|---|---|
| NOV46k, CG55794-04 | SEQ ID NO: 719 | 1579 bp |
| DNA Sequence | ORF Start: at 334 | ORF Stop: TAA at 1396 |

CCAATTGACAAGCTTTTGATTTTAACGACTTTTAACGACAACTTGAGAAGATCAAAAAACAACTAATT
ATTCGAAACGATGAGATTTCCTTCAATTTTTACTGCTGTTTTATTCGCAGCATCCTCCGCATTAGCTG
CTCCAGTCAACACTACAACAGAAGATGAAACGGCACAAATTCCGGCTGAAGCTGTCATCGGTTACTCA
GATTTAGAAGGGGATTTCGATGTTGCTGTTTTGCCATTTTCCAACAGCACAAATAACGGGTTATTGTT
TATAAATACTACTATTGCCAGCATTGCTGCTAAAGAAGAAGGGGTATCTCTCGAGAAAAGATATGATA
GATCCTTAGCCCAACACAGACAAGAGATTGTGGACAAGTCAGTGAGTCCATAAAGCCTGGAGACGTAT
TCCTATAACATATACCACCCCATGGGAGAGATCTATGAGTGGATGAGAGAGATCAGTGAGAAGTACAA
GGAAGTGGTGACACAGCATTTCCTAGGAGTGACCTATGAGACCCACCCCATGTATTATCTGAAGATAA
GCCAACCATCTGGTAATCCCAAGAAAATCATTTGGATGGACTGTGGAATTCACGCCAGAGAATGGATT
GCTCCTGCTTTTTGCCAATGGTTCGTCAAAGAAATTCTACAAAACCATAAAGACAACTCAAGTATACG
CAAGCTTCTTAGGAACCTGGACTTCTATGTCCTTCCAGTTCTTAACATAGATGGTTATATCTACACAA
GGACAACTGATCGTCTTTGGAGGAAATCCCGTTCACCCCATAATAATGGCACATGTTTTGGGACGGAT
CTCAATCGAAATTTCAATGCATCTTGGTGTAGTATTGGTGCCTCTAGAAACTGCCAAGATCAAACATT
CTGTGGGACAGGGCCAGTGTCTGAACCAGAGACTAAAGCTGTTGCCAGCTTCATAGAGAGCAAGAAGG
ATGATATTTTGTGCTTCCTGACCATGCACTCTTATGGGCAGTTAATTCTCACACCTTACGGAAACACC
AAAAATAAATCAAGTAACCACCCAGAAATGATTCAAGTTGGACAGAAGGCAGCAAATGCATTGAAAGC
AAAGTATGGAACCAATTATAGAGTTGGATCGAGTGCAGATATTTTATATGCCTCATCAGGGTCTTCAA
GAGATTGGGCCCGAGACATTGGGATTCCCTTCTCATATACGTTTGAGCTGAGGGACAGTGGAACGTAT
GGGTTTGTTCTGCCAGAAGCTCAGATCCAGCCCACCTGTGAGGAGACCATGGAGGCTGTGCTGTAAGT
CCTGGATGATGTGTATGCGAAACACTGGCACTCGGACAGTGCTGGAAGGGTGACATCTGCCACTATGC
TGCTGGGCCTGCTGGTGTCCTGCATGTCTCTTCTCTAAGCGGCCGCCAGCTTTCTAGAACAAAAACTC
ATCTCAGAAGAGGATCTGAATAGCGCCGTCGACCATCATCATCATCATCATTGAGTTTGTAGCCTTAG
ACATGACTGTTCCTCAGTTCAAGTTGGGCACTTACGAGAAGACCGGTCTTGCTAGATTCTAATCAAGA
GGATGTCAGAATGCC

| | | | |
|---|---|---|---|
| NOV46k, CG55794-04 | | | |
| Protein Sequence | SEQ ID NO: 720 | 354 aa | MW at 40431.3kD |

YDRSLAQHRQEIVDKSVSPWSLETYSYNIYHPMGEIYEWMREISEKYKEVVTQHFLGVTYETHPMYYL
KISQPSGNPKKIIWMDCGIHAREWIAPAFCQWFVKEILQNHKDNSSIRKLLRNLDFYVLPVLNIDGYI
YTWTTDRLWRKSRSPHNNGTCFGTDLNRNFNASWCSIGASRNCQDQTFCGTGPVSEPETKAVASFIES
KKDDILCFLTMHSYGQLILTPYGYTKNKSSNHPEMIQVGQKAANALKAKYGTNYRVGSSADILYASSG
SSRDWARDIGIPFSYTFELRDSGTYGFVLPEAQIQPTCEETMEAVLSVLDDVYAKWHSDSAGRAVTSA
TMLLGLLVSCMSLL

| | | |
|---|---|---|
| NOV461, CG55794-05 | SEQ ID NO: 721 | 1607 bp |
| DNA Sequence | ORF Start: at 341 | ORF Stop: at 1403 |

ACTGGTTCCAATTGACAAGCTTTTGATTTTAACGACTTTTAACGACAACTTGAGAAGATCAAAAAACA
ACTAATTATTCGAAACGATGAGATTTCCTTCAATTTTTACTGCTGTTTTATTCGCAGCATCCTCCGCA
TTAGCTGCTCCAGTCAACACTACAACAGAAGATGAAACGGCACAAATTCCGGCTGAAGCTGTCATCGG
TTACTCAGATTTAGAAGGGGATTTCGATGTTGCTGTTTTGCCATTTTCCAACAGCACAAATAACGGGT
TATTGTTTATAAATACTACTATTGCCAGCATTGCTGCTAAAGAAGAAGGGGTATCTCTCGAGAAAAGA
TATGATAGATCCTTAGCCCAACACAGACAAGAGATTGTGGACAAGTCAGTGAGTCCATGGAGCCTGGA
GACGTATTCCTATAACATATACCACCCCATGGGAGAGATCTATGAGTGGATGAGAGAGATCAGTGAGA
AGTACAAGGAAGTGGTGACACAGCATTTCCTAGGAGTGACCTATGAGACCCACCCCATGTATTATCTG
AAGATCAGCCAACCATCTGGTAATCCCAAGAAAATCATTTGGATGGACTGTGGAATTCACGCCAGAGA
ATGGATTGCTCCTGCTTTTTGCCAATGGTTCGTCAAAGAAATTCTACAAAACCATAAAGACAACTCAA
GTATACGCAAGCTTCTTAGGAACCTGGACTTCTATGTCCTTCCAGTTCTTAACATAGATGGTTATATC
TACACTTGGACAACTGATCGTCTTTGGAGGAAATCCCGTTCACCCCATAATAATGGCACATGTTTTGG
GACGGATCTCAATCGAAATTTCAATGCATCTTGGTGTAGTATTGGTGCCTCTAGAAACTGCCAAGATC
AAACATTCTGTGGGACAGGGCCAGTGTCTGAACCAGAGACTAAAGCTGTTGCCAGCTTCATAGAGAGC
AAGAAGGATGATATTTTGTGCTTCCTGACCATGCACTCTTATGGGCAGTTAATTCTCACACCTTACGG
CTACACCAAAAATAAATCAAGTAACCACCCAGAAATGATTCAAGTTGGACAGAAGGCAGCAAATGCAT
TGAAAGCAAAGTATGGAACCAATTATAGAGTTGGATCGAGTGCAGATATTTTATATGCCTCATCAGGG
TCTTCAAGAGATTGGGCCCGAGACATTGGGATTCCCTTCTCATATACGTTTGAGCTGAGGGACAGTGG
AACGTATGGGTTTGTTCTGCCAGAAGCTCAGATCCAGCCCACCTGTGAGGAGACCATGGAGGCTGTGC
TGTCAGTCCTGGATGATGTGTATGCGAAACACTGGCACTCGGACAGTGCTGGAAGGGTGACATCTGCC
ACTATGCTGCTGGGCCTGCTGGTGTCCTGCATGTCTCTTCTCCACCATCACCACCATCACTAAGCGGC
CGCCAGCTTTCTAGAACAAAAACTCATCTCAGAAGAGGATCTGAATAGCGCCGTCGACCATCATCATC
ATCATCATTGAGTTTGTAGCCTTAGACATGACTGTTCCTCAGTTCAAGTTGGGCACTTACGAGAAGAC
CGGTCTTGCTAGATTCTAATCAAGAGGATGTCAGAATGCCATT

| | | | |
|---|---|---|---|
| NOV461, CG55794-05 | | | |
| Protein Sequence | SEQ ID NO: 722 | 354 aa | MW at 40431.3kD |

YDRSLAQHRQEIVDKSVSPWSLETYSYNIYHPMGEIYEWMREISEKYKEVVTQHFLGVTYETHPMYYL
KISQPSGNPKKIIWMDCGIHAREWIAPAFCQWFVKEILQNHKDNSSIRKLLRNLDFYVLPVLNIDGYI
YTWTTDRLWRKSRSPHNNGTCFGTDLNRNFNASWCSIGASRNCQDQTFCGTGPVSEPETKAVASFIES
KKDDILCFLTMHSYGQLILTPYGYTKNKSSNHPEMIQVGQKAANALKAKYGTNYRVGSSADILYASSG
SSRDWARDIGIPFSYTFELRDSGTYGFVLPEAQIQPTCEETMEAVLSVLDDVYAKHWHSDSAGRVTSA
TMLLGLLVSCMSLL

| | | |
|---|---|---|
| NOV46m, GG55794-06 | SEQ ID NO: 723 | 1977 bp |
| DNA Sequence | ORF Start: ATG at 41 | ORF Stop: TAG at 671 |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGCTGGGATATGATAGATCCTTAGCCCAACACAGACAAGAGATT
GTGGACAAGTCAGTGAGTCCATGGAGCCTGGAAACGTATTCCTATAACATATACCACCCCATGGGAGA
GATCTATGAGTGGATGAGAGAGATCAGTGAGAAGTACAAGGAAGTGGTGACACAGCATTTCCTAGGAG
TGACCTATGAGACCCACCCATATATTATCTGAAGATCAGCCAACCATACTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAATTCAAGTTGGACAGAAGGCAGCAAATGCATTGAAAGCAAAGTATGGAACCAATTATAGAGTTGGA
TCGAGTGCAGATATTTTATATGCCTCATCAGGGTCTTCAAGAGATTGGGCCCGAGACATTGGGATTCC
CTTCTCATATACGTTTGAGCTGAGGGACAGTGGAACATATGGGTTTGTTCTGCCAGAAGCTCAGATCC
AGCCCACCTGTGAGGAGACCATGGAGGCTGTGCTGTCAGTCCTGGATGATGTGTATGCCGAACACTGG
CACTCGGACAGTGCTGGAAGGGTGACATCTGCCACTATGCTGTGGGCCTGTCTGGTGTCCTGCATGTC
TCTTCTCTAAGTGCATCCTGCCCAG

| | | | |
|---|---|---|---|
| NOV46m, CG55794-06 | | | |
| Protein Sequence | SEQ ID NO: 724 | 210 aa | MW at 24848.2kD |

MKPLLETLYLLGMLVPGGLGYDRSLAQHRQEIVDKSVSPWSLETYSYNIYHPMGEIYEWMREISEKYK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCNSSWTEGSKCIES
KVWNQL

| | | |
|---|---|---|
| NOV46n, CG55794-07 | SEQ ID NO: 725 | 1378 bp |
| DNA Sequence | ORF Start: ATG at 259 | ORF Stop: TAA at 1225 |

ACTCACTATAGGGCTCGAGCGGCCGCCCGGGCAGGTGAAAATACATCAGCATGTGGGAAAGAGCAACG
TTGATCGTCTTCACGGAAAGGCTGAGGACCCTGCGCCTACCACATGTTGGCCAGGGTGAGCAAGCAGT
GAAAGAGAAAACACTTTTTTCAAAAAGCCAACTGATCCTTAGCCCAACACAGACAAGAGATTGTGGAC
AAGTCAGTGAGTCCATGGAGCCTGGAGACGTATTCCTATAACATATACCACCCCATGGGAGAGATCTA
TGAGTGGATGAGAGAGATCAGTGAGAAGTACAAGGAAGTGGTGACACAGCATTTCCTAGGAGTGACCT
ATGAGACCCACCCCATATATTATCTGAAGATCAGCCAACCATCTGGTAATCCCAAGAAAATCATTTGG
ATGGACTGTGGAATTCACGCCAGAGAATGGATTGCTCCTGCTTTTTGCCAATGGTTCGTCAAAGAAAT
TCTACAAAACCATAAAGACAACTCAAGGATACGCAAGCTCCTTAGGAACCTGGACTTCTATGTCCTTC
CAGTTCTTAACATAGATGGTTATATCTACACTTGGACAACTGATCGTCTTTGGAGGAAATCCCGTTCA
CCCCATAATAATGGCACATGTTTTGGGACGGATCTCAATCGAAATTTCAATGCTTCTTGGTGTAGTAT
TGGTGCCTCTAGAAACTGCCAAGATCAAACATTCTGTGGGACAGGGCCAGTGTCTGAACCAGAGACTA
AAGCTGTTGCCAGCTTCATAGAGAGCAAGAAGGATGATATTTTGTGCTTCCTGACCATGCACTCTTAT
GGGCAGTTAATTCTCACACCTTACGGCTACACCAAAAATAAATCAAGTAACCACCCAGAAATGATTCA
AGTTGGACAGAAGGCAGCAAATGCATTGAAAGCAAAGTATGGAACCAATTATAGAGTTGGATCGAGTG
CAGATATTTTATATGCCTCATCAGGGTCTTCAAGAGATTGGGCCCGAGACATTGGGATTCCCTTCTCA
TATACGTTTGAGCTGAGGGACAGTGGAACATATGGGTTTGTTCTGCCAGAAGCTCAGATCCAGCCCAC
CTGTGAGGAGACCATGGAGGCTGTGCTGTCAGTCCTGGATGATGTGTATGCGAAACACTGGCACTCGG
ACAGTGCTGGAAGGGTGACATCTGCCACTATGCTGCTGGGCCTGCTGGTGTCCTGCATGTCTCTTCTC
TAAGTGCATTCTGCCCAGGCCTGCTCAACCCCAGTGGCATGAGTGTGGCTGGAGGAACGGTGTGTTAT
GGTTGTAAAGAAACCAAATAATTTAACTAAAAATACTTCCTATTTCAATAAAAAAAAAAAAAAAAAAA
AAAAAAAAAAAAA

| | | | |
|---|---|---|---|
| NOV46n, CG55794-07 | | | |
| Protein Sequence | SEQ ID NO: 726 | 1322 aa | MW at 36617.2kD |

MGEIYEWMREISEKYKEVVTQHFLGVTYETHPIYYLKISQPSGMPKKIIWMDCGIHAREWIAPAFCQW
FVKEILQNHKDNSRIRKLLRNLDFYVLPVLNIDGYIYTWTTDRLWRKSRSPHNNGTCFGTDLNRNFNA
SWCSIGASRNCQDQTFCGTGPVSEPETKAVASFIESKKDDILCFLTMHSYGQLILTPYGYTKNKSSNH
PEMIQVGQKAANALKAKYGTNYRVGSSADILYASSGSSRDWARDIGIPFSYTFELRDSGTYGFVLPEA
QIQPTCEETMEAVLSVLDDVYAKHWIISDSAGRVRSATMLLGLLVSCMSLL

| | | |
|---|---|---|
| NOV46o, CG55794-08 | SEQ ID NO: 727 | 128 bp |
| DNA Sequence | ORF Start: at 1 | ORF Stop: TAA at 1126 |

ACCATGAAGCCTCTGCTTGAAACCCTTTATCTTTTGGGGATGCTGGTTCCTGGAGGGCTGGGATATGA
TAGATCCTTAGCCCAACACAGACAAGAGATTGTGGACAAGTCAGTGAGTCCATGGAGCCTGGAGACGT
ATTCCTATAACATATACCACCCCATGGGAGAGATCTATGAGTGGATGAGAGAGATCAGTGAGAAGTAC
AAGGAAGTGGTGACACAGCATTTCCTAGGAGTGACCTATGAGACCCACCCCATGTATTATCTGAAGAT
CAGCCAACCATCTGGTAATCCCAAGAAAATCATTTGGATGGACTGTGGAATTCACGCCAGAGAATGGA
TTGCTCCTGCTTTTTGCCAATGGTTCGTCAAAGAAATTCTACAAAACCATAAAGACAACTCAAGTATA
CGCAAGCTTCTTAGGAACCTGGACTTCTATGTCCTTCCAGTTCTTAACATAGATGGTTATATCTACAC
TTGGACAACTGATCGTCTTTGGAGGAAATCCCGTTCACCCCATAATAATGGCACATGTTTTGGGACGG
ATCTCAATCGAAATTTCAATGCATCTTGGTGTAGTATTGGTGCCTCTAGAAACTGCCAAGATCAAACA
TTCTGTGGGACAGGGCCAGTGTCTGAACCAGAGACTAAAGCTGTTGCCAGCTTCATAGAGAGCAAGAA
GGATGATATTTTGTGCTTCCTGACCATGCACTCTTATGGGCAGTTAATTCTCACACCTTACGGCTACA
CCAAAAATAATCAAGTAACCACCCAGAAATGATTCAAAGTTGGACAGAAGGCAGCAAATGCATTGAAA
GCAAAGTATGGAACCAATTATAGAGTTGGATCGAGTGCAGATATTTTATATGCCTCATCAGGGTCTTC
AAGAGATTGGGCCCGAGACATTGGGATTCCCTTCTCATATACGTTTGAGCTGAGGGACAGTGGAACGT
ATGGGTTTGTTCTGCCAGAAGCTCAGATCCAGCCCACCTGTGAGGAGACCATGGAGGCTGTGCTGTCA
GTCCTGGATGATGTGTATGCGAAACACTGGCACTCGGACAGTGCTGGAAGGGTGACATCTGCCACTAT
GCTGCTGGGCCTGCTGGTGTCCTGCATGTCTCTTCTCTAA

| | | | |
|---|---|---|---|
| NOV46o, CG55794-08 | | | |
| Protein Sequence | SEQ ID NO: 728 | 375 aa | MW at 42630.1kD |

TMKPLLETLYLLGMLVPGGLGYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLL

| | | |
|---|---|---|
| NOV46p, CG55794-09 | SEQ ID NO: 729 | 1151 bp |
| DNA Sequence | ORF Start: at 3 | ORF Stop: TAA at 1149 |

CCACCATGGGCCACCATCACCACCATCACAAGCCTCTGCTTGAAACCCTTTATCTTTTGGGGATGCTG
GTTCCTGGAGGGCTGGGATATGATAGATCCTTAGCCCAACACAGACAAGAGATTGTGGACAAGTCAGT
GAGTCCATGGAGCCTGGAGACGTATTCCTATAACATATACCACCCCATGGGAGAGATCTATGAGTGGA
TGAGAGAGATCAGTGAGAAGTACAAGGAAGTGGTGACACAGCATTTCCTAGGAGTGACCTATGAGACC
CACCCCATGTATTATCTGAAGATCAGCCAACCATCTGGTAATCCCAAGAAAATCATTTGGATGGACTG
TGGAATTCACGCCAGAGAATGGATTGCTCCTGCTTTTTGCCAATGGTTCGTCAAAGAAATTCTACAAA
ACCATAAAGACAACTCAAGTATACGCAAGCTTCTTAGGAACCTGGACTTCTATGTCCTTCCAGTTCTT
AACATAGATGGTTATATCTACACTTGGACAACTGATCGTCTTTGGAGGAAATCCCGTTCACCCCATAA
TAATGGCACATGTTTTGGGACGGATCTCAATCGAAATTTCAATGCATCTTGGTGTAGTATTGGTGCCT
CTAGAAACTGCCAAGATCAAACATTCTGTGGGACAGGGCCAGTGTCTGAACCAGAGACTAAAGCTGTT
GCCAGCTTCATAGAGAGCAAGAAGGATGATATTTTGTGCTTCCTGACCATGCACTCTTATGGGCAGTT
AATTCTCACACCTTACGGCTACACCAAAAATAAATCAAGTAACCACCCAGAAATGATTCAAGTTGGAC
AGAAGGCAGCAAATGCATTGAAAGCAAAGTATGGAACCAATTATAGAGTTGGATCGAGTGCAGATATT
TTATATGCCTCATCAGGGTCTTCAAGAGATTGGGCCCGAGACATTGGGATTCCCTTCTCATATACGTT
TGAGCTGAGGGACAGTGGAACGTATGGGTTTGTTCTGCCAGAAGCTCAGATCCAGCCCACCTGTGAGG
AGACCATGGAGGCTGTGCTGTCAGTCCTGGATGATGTGTATGCGAAACACTGGCACTCGGACAGTGCT
GGAAGGGTGACATCTGCCACTATGCTGCTGGGCCTGCTGGTGTCCTGCATGTCTCTTCTCTAA

| | | | |
|---|---|---|---|
| NOV46p, CG55794-09 | | | |
| Protein Sequence | SEQ ID NO: 730 | 382 aa | MW at 43510.0kD |

TMGHHHHHHKPLLETLYLLGMLVPGGLGYDRSLAQHRQEIVDKSVSPWSLETYSYNIYHPMGEIYEWM
REISEKYKEVVTQHFLGVTYETHPMYYLKISQPSGNPKKIIWMDCGIHAREWIAPAFCQWFVKEILQN
HKDNSSIRKLLRNLDFYVLPVLNIDGYIYTWTTDRLWRKSRSPHNNGTCFGTDLNRNFNASWCSIGAS
RNCQDQTFCGTGPVSEPETKAVASFIESKKDDILCFLTMHSYGQLILTPYGYTKNKSSNHPEMIQVGQ
KAANALKAKYGTNYRVGSSADILYASSGSSRDWARDIGIPFSYTFELRDSGTYGFVLPEAQIQPTCEE
TMEAVLSVLDDVYAKHWHSDSAGRVTSATMLLGLLVSCMSLL

| | | |
|---|---|---|
| NOV46q, CG55794-10 | SEQ ID NO: 731 | 1148 bp |
| DNA Sequence | ORF Start: at 3 | ORF Stop: TAA at 1146 |

CCACCATGAAGCCTCTGCTTGAAACCCTTTATCTTTTGGGGATGCTGGTTCCTGGAGGGCTGGGATAT
GATAGATCCTTAGCCCAACACAGACAAGAGATTGTGGACAAGTCAGTGAGTCCATGGAGCCTGGAGAC
GTATTCCTATAACATATACCACCCCATGGGAGAGATCTATGAGTGGATGAGAGAGATCAGTGAGAAGT
ACAAGGAAGTGGTGACACAGCATTTCCTAGGAGTGACCTATGAGACCCACCCCATGTATTATCTGAAG
ATCAGCCAACCATCTGGTAATCCCAAGAAAATCATTTGGATGGACTGTGGAATTCACGCCAGAGAATG
GATTGCTCCTGCTTTTTGCCAATGGTTCGTCAAAGAAATTCTACAAAACCATAAAGACAACTCAAGTA
TACGCIAGCTTCTTAGGAACCTGGACTTCTATGTCCTTCCAGTTCTTAACATAGATGGTTATATCTAC
ACTTGGACAACTGATCGTCTTTGGAGGAAATCCCGTTCACCCCATAATAATGGCACATGTTTTGGGAC
GGATCTCAATCGAAATTTCAATGCATCTTGGTGTAGTATTGGTGCCTCTAGAAACTGCCAAGATCAAA
CATTCTGTGGGACAGGGCCAGTGTCTGAACCAGAGACTAAAGCTGTTGCCAGCTTCATAGAGAGCAAG
AAGGATGATATTTTGTGCTTCCTGACCATGCACTCTTATGGGCAGTTAATTCTCACACCTTACGGCTA
CACCAAAAATAAATCAAGTAACCACCCAGAAATGATTCAAGTTGGACAGAAGGCAGCAAATGCATTGA
AAGCAAAGTATGGAACCAATTATAGAGTTGGATCGAGTGCAGATATTTTATATGCCTCATCAGGGTCT
TCAAGAGATTGGGCCCGAGACATTGGGATTCCCTTCTCATATACGTTTGAGCTGAGGGACAGTGGAAC
GTATGGGTTTGTTCTGCCAGAAGCTCAGATCCAGCCCACCTGTGAGGAGACCATGGAGGCTGTGCTGT
CAGTCCTGGATGATGTGTATGCGAAACACTGGCACTCGGACAGTGCTGGAAGGGTGACATCTGCCACT
ATGCTGCTGGGCCTGCTGGTGTCCTGCATGTCTCTTCTCCACCATCACCACCATCACTAA

| | | | |
|---|---|---|---|
| NOV46q, CG55794-10 | | | |
| Protein Sequence | SEQ ID NO: 732 | 381 aa | MW at 43452.9kD |

TMKPLLETLYLLGMLVPGGLGYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLLHHHHHH

| | | |
|---|---|---|
| NOV46r,CG55794-11 | SEQ ID NO: 733 | 1146 bp |
| DNA Sequence | ORF Start: at 1 | ORF Stop: TAA at 1144 |

ACCATGGTAAGCGCTATTGTTTTATATGTGCTTTTGGCGGCGGCGGCGCATTCTGCCTTTGCGTATGA
TAGATCCTTAGCCCAACACAGACAAGAGATTGTGGACAAGTCAGTGAGTCCATGGAGCCTGGAGACGT
ATTCCTATAACATATACCACCCCATGGGAGAGATCTATGAGTGGATGAGAGAGATCAGTGAGAAGTAC
AAGGAAGTGGTGACACAGCATTTCCTAGGAGTGACCTATGAGACCCACCCCATGTATTATCTGAAGAT
CAGCCAACCATCTGGTAATCCCAAGAAAATCATTTGGATGGACTGTGGAATTCACGCCAGAGAATGGA
TTGCTCCTGCTTTTTGCCAATGGTTCGTCAAAGAAATTCTACAAAACCATAAAGACAACTCAAGTATA
CGCAAGCTTCTTAGGAACCTGGACTTCTATGTCCTTCCAGTTCTTAACATAGATGGTTATATCTACAC
TTGGACAACTGATCGTCTTTGGAGGAAATCCCGTTCACCCCATAATAATGGCACATGTTTTGGGACGG
ATCTCAATCGAAATTTCAATGCATCTTGGTGTAGTATTGGTGCCTCTAGAAACTGCCAAGATCAAACA
TTCTGTGGGACAGGGCCAGTGTCTGAACCAGAGACTAAAGCTGTTGCCAGCTTCATAGAGAGCAAGAA
GGATGATATTTTGTGCTTCCTGACCATGCACTCTTATGGGCAGTTAATTCTCACACCTTACGGCTACA
CCAAAAATAAATCAAGTAACCACCCAGAAATGATTCAAGTTGGACAGAAGGCAGCAAATGCATTGAAA
GCAAAGTATGGAACCAATTATAGAGTTGGATCGAGTGCAGATATTTTATATGCCTCATCAGGGTCTTC
AAGAGATTGGGCCCGAGACATTGGGATTCCCTTCTCATATACGTTTGAGCTGAGGGACAGTGGAACGT
ATGGGTTTGTTCTGCCAGAAGCTCAGATCCAGCCCACCTGTGAGGAGACCATGGAGGCTGTGCTGTCA
GTCCTGGATGATGTGTATGCGAAACACTGGCACTCGGACAGTGCTGGAAGGGTGACATCTGCCACTAT
GCTGCTGGGCCTGCTGGTGTCCTGCATGTCTCTTCTCCACCATCACCACCATCACTAA

| | | | |
|---|---|---|---|
| NOV46r, CG55794-11 | | | |
| Protein Sequence | SEQ ID NO: 734 | 381 aa | MW at 43355.7kD |

TMVSAIVLYVLLAAAAHSAFAYDRSLAQHRQEIVDKSVSPWSLETYSYNIYHPMGEIYEWMREISEKY
KEVVTQHFLGVTYETHPMYYLKISQPSGNPKKIIWMDCGIHAREWIAPAFCQWFVKEILQNHKDNSSI
RKLLRNLDFYVLPVLNIDGYIYTWTTDRLWRKSRSPHNNGTCFGTDLNRNFNASWCSIGASRNCQDQT
FCGTGPVSEPETKAVASFIESKKDDILCFLTMHSYGQLILTPYGYTKNKSSNHPEMIQVGQKAANALK
AKYGTNYRVGSSADILYASSGSSRDWARDIGIPFSYTFELRDSGTYGFVLPEAQIQPTCEETMEAVLS
VLDDVYAKHWHSDSAGRVTSATMLLGLLVSCMSLLHHHHHH

| | | |
|---|---|---|
| NOV46s, CG55794-12 | SEQ ID NO: 735 | 1161 bp |
| DNA Sequence | ORF Start: at 1 | ORF Stop: TAA at 1159 |

ATTATTATACCTCCCACCATCGGGCGCGGATCCACCATGGTAAGCGCTATTGTTTTATATGTGCTTTT
GGCGGCGGCGGCGCATTCTGCCTTTGCGTATGATAGATCCTTAGCCCAACACAGACAAGAGATTGTGG
ACAAGTCAGTGAGTCCATGGAGCCTGGAGACGTATTCCTATAACATATACCACCCCATGGGAGAGATC
TATGAGTGGATGAGAGAGATCAGTGAGAAGTACAAGGAAGTGGTGACACAGCATTTCCTAGGAGTGAC
CTATGAGACCCACCCCATGTATTATCTGAAGATCAGCCAACCATCTGGTAATCCCAAGAAAATCATTT
GGATGGACTGTGGAATTCACGCCAGAGAATGGATTGCTCCTGCTTTTTGCCAATGGTTCGTCAAAGAA
ATTCTACAAAACCATAAAGACAACTCAAGTATACGCAAGCTTCTTAGGAACCTGGACTTCTATGTCCT
TCCAGTTCTTAACATAGATGGTTATATCTACACTTGGACAACTGATCGTCTTTGGAGGAAATCCCGTT
CACCCCATAATAATGGCACATGTTTTGGGACGGATCTCAATCGAAATTTCAATGCATCTTGGTGTAGT
ATTGGTGCCTCTAGAAACTGCCAAGATCAAACATTCTGTGGGACAGGGCCAGTGTCTGAACCAGAGAC
TAAAGCTGTTGCCAGCTTCATAGAGAGCAAGAAGGATGATATTTTGTGCTTCCTGACCATGCACTCTT
ATGGGCAGTTAATTCTCACACCTTACGGCTACACCAAAAATAAATCAAGTAACCACCCAGAAATGATT
CAAGTTGGACAGAAGGCAGCAAATGCATTGAAAGCAAAGTATGGAACCAATTATAGAGTTGGATCGAG
TGCAGATATTTTATATGCCTCATCAGGGTCTTCAAGAGATTGGGCCCGAGACATTGGGATTCCCTTCT
CATATACGTTTGAGCTGAGGGACAGTGGAACGTATGGGTTTGTTCTGCCAGAAGCTCAGATCCAGCCC
ACCTGTGAGGAGACCATGGAGGCTGTGGAACGTATGGGTTTGTTCTGCCAGAAGCTCAGATCCAGCCC
GGACAGTGCTGGAAGGGTGACATCTGCCACTATGCTGCTGGGCCTGCTGGTGTCCTGCATGTCTCTTC
TCTAA

| | | | |
|---|---|---|---|
| NOV46s, CG55794-12 | | | |
| Protein Sequence | SEQ ID NO: 736 | 386 aa | MW at 43638.1kD |

IIIPPTIGRGSTMVSAIVLYVLLAAAAHSAFAYDRSLAQHRQEIVDKSVSPWSLETYSYNIYHPMGEI
YEWMRBISEKYKEVVTQHFLGVTYETHPMYYLKISQPSGNPKKIIWMDCGIHAREWIAPAFCQWFVKE
ILQNHKDNSSIRKLLRNLDFYVLPVLNIDGYIYTWTTDRLWRKSRSPHNNGTCFGTDLNRNFNASWCS
IGASRNCQDQTFCGTGPVSEPETKAVASFIESKKDDILCFLTMHSYGQLILTPYGYTKNKSSNHPEMI
QVGQKAANALKAKYGTNYRVGSSADILYASSGSSRDWARDIGIPFSYTFELRDSGTYGFVLPEAQIQP
TCEETMEAVLSVLDDVYAKHWHSDSAGRVTSATMLLGLLVSCMSLL

| | | |
|---|---|---|
| NOV46t, CG55794-13 | SEQ ID NO: 737 | 1212 bp |
| DNA Sequence | ORF Start: at 1 | ORF Stop: TAA at 1210 |

CGCGGATCCACCATGGTAAGCGCTATTGTTTTATATGTGCTTTTGGCGGCGGCGGCGCATTCTGCCTT
TGCGAAGCCTCTGCTTGAAACCCTTTATCTTTTGGGGATGCTGGTTCCTGGAGGGCTGGGATATGATA
GATCCTTAGCCCAACACAGACAAGAGATTGTGGACAAGTCAGTGAGTCCATGGAGCCTGGAGACGTAT
TCCTATAACATATACCACCCCATGGGAGAGATCTATGAGTGGATGAGAGAGATCAGTGAGAAGTACAA
GGAAGTGGTGACACAGCATTTCCTAGGAGTGACCTATGAGACCCACCCCATGTATTATCTGAAGATCA
GCCAACCATCTGGTAATCCCAAGAAAATCATTTGGATGGACTGTGGAATTCACGCCAGAGAATGGATT
GCTCCTGCTTTTTGCCAATGGTTCGTCAAAGAAATTCTACAAAACCATAAAGACAACTCAAGTATACG
CAAGCTTCTTAGGAACCTGGACTTCTATGTCCTTCCAGTTCTTAACATAGATGGTTATATCTACACTT
GGACAACTGATCGTCTTTGGAGGAAATCCCGTTCACCCCATAATAATGGCACATGTTTTGGGACGGAT
CTCAATCGAAATTTCAATGCATCTTGGTGTAGTATTGGTGCCTCTAGAAACTGCCAAGATCAAACATT
CTGTGGGACAGGGCCAGTGTCTGAACCAGAGACTAAAGCTGTTGCCAGCTTCATAGAGAGCAAGAAGG
ATGATATTTTGTGCTTCCTGACCATGCACTCTTATGGGCAGTTAATTCTCACACCTTACGGCTACACC
AAAAATAAATCAAGTAACCACCCAGAAATGATTCAAGTTGGACAGAAGGCAGCAAATGCATTGAAAGC
AAAGTATGGAACCAATTATAGAGTTGGATCGAGTGCAGATATTTTATATGCCTCATCAGGGTCTTCAA
GAGATTGGGCCCGAGACATTGGGATTCCCTTCTCATATACGTTTGAGCTGAGGGACAGTGGAACGTAT
GGGTTTGTTCTGCCAGAAGCTCAGATCCAGCCCACCTGTGAGGAGACCATGGAGGCTGTGCTGTCAGT
CCTGGATGATGTGTATGCGAAACACTGGCACTCGGACAGTGCTGGAAGGGTGACATCTGCCACTATGC
TGCTGGGCCTGCTGGTGTCCTGCATGTCTCTTCTCCACCATCACCACCATCACTAA

| | | | |
|---|---|---|---|
| NOV46t, CG55794-13 | | | |
| Protein Sequence | SEQ ID NO: 738 | 403 aa | MW at 45622.4kD |

RGSTMVSAIVLYVLLAAAAHSAFAKPLLETLYLLGMLVPGGLGYDRSLAQHRQEIVDKSVSPWSLETY
SYNIYHPMGEIYEWMREISEKYKEVVTQHFLGVTYETHPMYYLKISQPSGNPKKIIWMDCGIHAREWI
APAFCQWFVKEILQNHKDNSSIRKLLRNLDFYVLPVINIDGYIYTWTTDRLWRKSRSPHNNGTCFGTD
LNRNFNASWCSIGASRNCQDQTFCGTGPVSEPETKAVASFIESKKDDILCFLTMHSYGQLILTPYGYT
KNKSSNHPEMIQVGQKAANALKAKYGTNYRVGSSADILYASSGSSRDWARDIGIPFSYTFELRDSGTY
GFVLPEAQIQPTCEETMEAVLSVLDDVYAKHWHSDSAGRVTSATMLLGLLVSCMSLLHHHHHH

| | | |
|---|---|---|
| NOV46u, | | |
| SNP13375362 of | SEQ ID NO: 739 | 1222 bp |
| CG55794-03, | ORF Start: ATG at 41 | ORF Stop: TAA at 1163 |
| DNA Sequence SNP Pos: 240 | SNP Change: A to G | |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGCTGGGATATGATAGATCCTTAGCCCAACACAGACAAGAGATT
GTGGACAAGTCAGTGAGTCCATGGAGCCTGGAGACGTATTCCTATAACATATACCACCCCATGGGAGA
GATCTATGAGTGGATGAGAGAGATCAGTGAGAAGTGCAAGGAAGTGGTGACACAGCATTTCCTAGGAG
TGACCTATGAGACCCACCCCATATATTATCTGAAGATCAGCCAACCATCTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAGTATTGGTGCCTCTAGAAACTGCCAAGATCAAACATTCTGTGGGACAGGGCCAGTGTCTGAACCAG
TAGACTAAGCTGTTGCCAGCTTCATAGAGAGCAAGAAGGATGATATTTTGTGCTTCCTGACCATGCAC
TCTTATGGGCAGTTAATTCTCACACCTTACGGCTACACCAAAAATAAATCAAGTAACCACCCAGAAAT
GATTCAAGTTGGACAGAAGGCAGCAAATGCATTGAAAGCAAAGTATGGAACCAATTATAGAGTTGGAT
CGAGTGCAGATATTTTATATGCCTCATCAGGGTCTTCAAGAGATTGGGCCCGAGACATTGGGATTCCC
TTCTCATATACGTTTGAGCTGAGGGACAGTGGAACATATGGGTTTGTTCTGCCAGAAGCTCAGATCCA
GCCCACCTGTGAGGAGACCATGGAGGCTGTGCTGTCAGTCCTGGATGATGTGTATGCGAAACACTGGC
ACTCGGACAGTGCTGGAAGGGTGACATCTGCCACTATGCTGCTGGGCCTGCTGGTGTCCTGCATGTCT
CTTCTCTAAGTGCATTCTGCCCAGGCCTGCTCAACCCCAGTGGCATGAGTGTGGCTTGGAGGAACG

| | | | |
|---|---|---|---|
| NOV46u, | | | |
| SNP13375362 of | | | |
| CG55794-03, | SEQ ID NO: 740 | | MW at 42520.0kD |
| Protein Sequence | SNP Pos: 67 | 374 aa | SNP Change: Tyr to Cys |

MKPLLETLYLLGMLVPGGLGYDRSLAQHRQEIVDKSVSPWSLETYSYNIYHPMGEIYEWMREISEKCK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPVSEPETKAVASFIESKKDDILCFLTMHSYGQLILTPYGYTKNKSSNHPEMIQVGQKAANALKA
KYGTNYRVGSSADILYASSGSSRDWARDIGIPFSYTFELRDSGTYGFVLPEAQIQPTCEETMEAVLSV
LDDVYAKHWHSDSAGRVTSATMLLGLLVSCMSLL

| | | |
|---|---|---|
| NOV46v, | | |
| SNP13379598 of | SEQ ID NO: 741 | 1222 bp |
| CG55794-03, | ORF Start: ATG at 41 | ORF Stop: TAA at 1163 |
| DNA Sequence | SNP Pos: 988 | SNP Change: A to G |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGCTGGGATATGATAGATCCTTAGCCCAACACAGACAAGAGATT
GTGGACAAGTCAGTGAGTCCATGGAGCCTGGAGACGTATTCCTATAACATATACCACCCCATGGGAGA
GATCTATGAGTGGATGAGAGAGATCAGTGAGAAGTACAAGGAAGTGGTGACACAGCATTTCCTAGGAG
TGACCTATGAGACCCACCCCATATATTATCTGAAGATCAGCCAACCATCTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAGTATTGGTGCCTCTAGAAACTGCCAAGATCAAACATTCTGTGGGACAGGGCCAGTGTCTGAACCAG
AGACTAAAGCTGTTGCCAGCTTCATAGAGAGCAAGAAGGATGATATTTTGTGCTTCCTGACCATGCAC
TCTTATGGGCAGTTAATTCTCACACCTTACGGCTACACCAAAAATAAATCAAGTAACCACCCAGAAAT
GATTCAAGTTGGACAGAAGGCAGCAAATGCATTGAAAGCAAAGTATGGAACCAATTATAGAGTTGGAT
CGAGTGCAGATATTTTATATGCCTCATCAGGGTCTTCAAGAGATTGGGCCCGAGACATTGGGATTCCC
TTCTCATATACGTTTGAGCTGAGGGACAGTGGAACGTATGGGTTTGTTCTGCCAGAAGCTCAGATCCA
GCCCACCTGTGAGGAGACCATGGAGGCTGTGCTGTCAGTCCTGGATGATGTGTATGCGAAACACTGGC
ACTCGGACAGTGCTGGAAGGGTGACATCTGCCACTATGCTGCTGGGCCTGCTGGTGTCCTGCATGTCT
CTTCTCTAAGTGCATTCTGCCCAGGCCTGCTCAACCCCAGTGGCATGAGTGTGGCTTGGAGGAACG

| | | | |
|---|---|---|---|
| NOV46v, | | | |
| SNP13379598 of | | | |
| CG55794-03, | SEQ ID NO: 742 | | MW at 42580.OkD |
| Protein Sequence | SNP Pos: 316 | 374 aa | SNP Change: Thr to Thr |

MKPLLETLYLLGMLVPGGLGYDRSLAQHRQEIVDKSVSPWSLETYSYNIYHPMGEIYEWMREISEKYK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPVSEPETKAVASFIESKKDDILCFLTMHSYGQLILTPYGYTKNKSSNHPEMIQVGQKAANALKA
KYGTNYRVGSSADILYASSGSSRDWARDIGIPFSYTFELRDSGTYGFVLPEAQIQPTCEETMEAVLSV
LDDVYAKHWHSDSAGRVTSATMLLGLLVSCNSLL

| | | |
|---|---|---|
| NOV46w, | | |
| SNP13375066 of | SEQ ID NO: 743 | 1222 bp |
| CG55794-03, | ORF Start: ATG at 41 | ORF Stop: TAA at 1163 |
| DNA Sequence | SNP Pos: 1152 | SNP Change: T to C |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGCTGGGATATGATAGATCCTTAGCCCAACACAGACAAGAGATT
GTGGACAAGTCAGTGAGTCCATGGAGCCTGGAGACGTATTCCTATAACATATACCACCCCATGGGAGA
GATCTATGAGTGGATGAGAGAGATCAGTGAGAAGTACAAGGAAGTGGTGACACAGCATTTCCTAGGAG
TGACCTATGAGACCCACCCCATATATTATCTGAAGATCAGCCAACCATCTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAGTATTGGTGCCTCTAGAAACTGCCAAGATCAAACATTCTGTGGGACAGGGCCAGTGTCTGAACCAG
AGACTAAAGCTGTTGCCAGCTTCATAGAGAGCAAGAAGGATGATATTTTGTGCTTCCTGACCATGCAC
TCTTATGGGCAGTTAATTCTCACACCTTACGGCTACACCAAAAATAAATCAAGTAACCACCCAGAAAT
GATTCAAGTTGGACAGAAGGCAGCAAATGCATTGAAAGCAAAGTATGGAACCAATTATAGAGTTGGAT
CGAGTGCAGATATTTTATATGCCTCATCAGGGTCTTCAAGAGATTGGGCCCGAGACATTGGGATTCCC
TTCTCATATACGTTTGAGCTGAGGGACAGTGGGAACATATGGGTTTGTTCTGCCAGAGCTCAGATCCA
GCCCACCTGTGAGGAGACCATGGAGGCTGTGCTGTCAGTCCTGGATGATGTGTATGCGAAACACTGGC
ACTCGGACAGTGCTGGAAGGGTGACATCTGCCACTATGCTGCTGGGCCTGCTGGTGTCCTGCACGTCT
CTTCTCTAAGTGCATTCTGCCCAGGCCTGCTCAACCCCAGTGGCATGAGTGTGGCTTGGAGGAACG

| | | | |
|---|---|---|---|
| NOV46w, | | | |
| SNP13375066 of | | | |
| CG55794-03, | SEQ ID NO: 744 | | MW at 42549.9kD |
| Protein Sequence | SNP Pos: 371 | 374 aa | SNP Change: Met to Thr |

MKPLLETLYLLGMLVPGGLGYDRSLAQHRQEIVDKSVSPWSLETYSYNIYHPMGEIYEWMREISEKYK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPAVSEPETKAVASFIESKKDDILCFLTHSYGQLILTPYGYTKNKSSNHPEMIQVGQKAANALKA
KYGTNYRVGSSADILYASSGSSRDWARDIGIPFSYTFELRDSGTYGFVLPEAQIQPTCEETMEAVLSV
LDDVYAKHWHSDSAGRVTSATMLLGLLVSCTSLL

| | | |
|---|---|---|
| NOV46x, | | |
| SNP13375067 of | SEQ ID NO: 745 | 1222 bp |
| CG55794-03, | ORF Start: ATG at 41 | ORF Stop: TAA at 1163 |
| DNA Sequence | SNP Pos: 1161 | SNP Change: T to C |

CCAGAGAGGCCCAGAATTTTCTAACTTACTGTGTGGCAGAATGAAGCCTCTGCTTGAAACCCTTTATC
TTTTGGGGATGCTGGTTCCTGGAGGGTGGGATATGATAGAATCCTTAGCCCAACACAGACAAGAGATT
GTGGACAAGTCAGTGAGTCCATGGAGCCTGGAGACGTATTCCTATAACATATACCACCCCATGGGAGA
GATCTATGAGTGGATGAGAGAGATCAGTGAGAAGTACAAGGAAGTGGTGACACAGCATTTCCTAGGAG
TGACCTATGAGACCCACCCCATATATTATCTGAAGATCAGCCAACCATCTGGTAATCCCAAGAAAATC
ATTTGGATGGACTGTGGAATTCACGCCAGAGAATGGATTGCTCCTGCTTTTTGCCAATGGTTCGTCAA
AGAAATTCTACAAAACCATAAAGACAACTCAAGGATACGCAAGCTCCTTAGGAACCTGGACTTCTATG
TCCTTCCAGTTCTTAACATAGATGGTTATATCTACACTTGGACAACTGATCGTCTTTGGAGGAAATCC
CGTTCACCCCATAATAATGGCACATGTTTTGGGACGGATCTCAATCGAAATTTCAATGCTTCTTGGTG
TAGTATTGGTGCCTCTAGAAACTGCCAAGATCAAACATTCTGTGGGACAGGGCCAGTGTCTGAACCAG
AGACTAAAGCTGTTGCCAGCTTCATAGAGAGCAAGAAGGATGATATTTTGTGCTTCCTGACCATGCAC
TCTTATGGGCAGTTAATTCTCACACCTTACGGCTACACCAAAAATAAATCAAGTAACCACCCAGAAAT
GATTCAAGTTGGACAGAAGGCAGCAAATGCATTGAAAGCAAAGTATGGAACCAATTATAGAGTTGGAT
CGAGTGCAGATATTTTATATGCCTCATCAGGGTCTTCAAGAGATTGGGCCCGAGACATTGGGATTCCC
TTCTCATATACGTTTGAGCTGAGGGACAGTGGAACATATGGGTTTGTTCTGCCAGAAGCTCAGATCCA
GCCCACCTGTGAGGAGACCATGGAGGCTGTGCTGTCAGTCCTGGATGATGTGTATGCGAAACACTGGC
ACTCGGACAGTGCTGGAAGGGTGACATCTGCCACTATGCTGCTGGGCCTGCTGGTGTCCTGCATGTCT
CTTCCCTAAGTGCCTGCCCAGGCCTGCTCAACCCCAGTGGCATGAGTGTGGCTTGGAGGAACG

| | | | |
|---|---|---|---|
| NOV46x, | | | |
| SNP13375067 of | | | |
| CG55794-03, | SEQ ID NO: 746 | | MW at 42564.0kD |
| Protein Sequence | SNP Pos: 374 | 374 aa | SNP Change: Leu to Pro |

MKPLLETLYLLGMLVPGGLGYDRSLAQHRQEIVDKSVSPWSLETYSYNIYHPMGEIYEWMREISEKYK
EVVTQHFLGVTYETHPIYYLKISQPSGNPKKIIWMDCGIHAREWIAPAFCQWFVKEILQNHKDNSRIR
KLLRNLDFYVLPVLNIDGYIYTWTTDRLWRKSRSPHNNGTCFGTDLNRNFNASWCSIGASRNCQDQTF
CGTGPVSEPETKAVASFIESKKDDILCFLTMHSYGQLILTPYGYTKNKSSNHPEMIQVGQKAANALKA
KYGTNYRVGSSADILYASSGSSRDWARDIGIPFSYTFELRDSGTYGFVLPEAQIQPTCEETMEAVLSV
LDDVYAKHWHSDSAGRVTSATMLLGLLVSCMSLP

A ClustalW comparison of the above protein sequences yields the following sequence alignment shown in Table 46B.

TABLE 46B

Comparison of the NOV46 protein sequences.

| | |
|---|---|
| NOV46a | ------------------------------------------------------------ |
| NOV46b | LFETMRFPSIFTAVLFAASSALAAPVNTTTEDETAQIPAEAVIGYSDLEGDFDVAVLPFS |
| NOV46c | LFETMRFPSIFTAVLFAASSALAAPVNTTTEDETAQIPAEAVIGYSDLEGDFDVAVLPFS |
| NOV46d | ------------------------------------------------------------ |
| NOV4Ee | ------------------------------------------------------------ |
| NOV46f | ----------------------------------------------------------TM |
| NOV46g | ----------------------------------------------------------TM |
| NOV46h | ------------------------------------------------------------ |
| NOV46i | ------------------------------------------------------------ |
| NOV46j | ------------------------------------------------------------ |
| NOV46k | ------------------------------------------------------------ |
| NOV46l | ------------------------------------------------------------ |
| NOV46m | ------------------------------------------------------------ |
| NOV46n | ------------------------------------------------------------ |
| NOV46o | ------------------------------------------------------------ |
| NOV46p | ----------------------------------------------------------TM |
| NOV46q | ------------------------------------------------------------ |
| NOV46r | ------------------------------------------------------------ |
| NOV46s | -----------------------------------------------------IIIPPTI |
| NOV46t | ------------------------------------------------------------ |
| NOV46a | ------MKPLLETLYLLGMLVPG------------------GLGYDRSLAQHRQEIVDKS |
| NOV46b | NSTNNGLLFINTTIASIAAKEEGVSL---------------EKRYDRSLAQHRQEIVDKS |
| NOV46c | NSTNNGLLFINTTIASIAAKEEGVSL---------------EKRYDRSLAQHRQEIVDKS |
| NOV46d | ----TMKPLLETLYLLGMLVPGG-------------------LGYDRSLAQHRQEIVDKS |
| NOV46e | ----TMKPLLETLYLLGMLVPGG-------------------LGYDRSLAQHRQEIVDKS |
| NOV46f | GHHHHHHKPLLETLYLLGMLVPG------------------GLGYDRSLAQHRQEIVDKS |
| NOV46g | GHHHHHHKPLLETLYLLGMLVPG------------------GLGYDRSLAQHRQEIVDKS |
| NOV46h | GHHHHHHKPLLETLYLLGMLVPG------------------GLGYDRSLAQHROEIVDKS |
| NOV46i | ------MKPLLETLYLLGMLVPG------------------GLGYDRSLAQHRQEIVDKS |
| NOV46j | --------------------------------------------YDRSLAQHRQEIVDKS |
| NOV46k | --------------------------------------------YDRSLAQHRQEIVDKS |
| NOV46l | --------------------------------------------YDRSLAQHRQEIVDKS |
| NOV46m | ------MKPLLETLYLLGMLVPG------------------GLGYDRSLAQHRQEIVDKS |
| NOV46n | ------------------------------------------------------------ |
| NOV46o | ----TMKPLLETLYLLGMLVPGG-------------------LGYDRSLAQHRQEIVDKS |
| NOV46p | GHHHHHHKPLLETLYLLGMLVPG------------------GLGYDRSLAQHRQEIVDKS |
| NOV46q | ----TMKPLLETLYLLGMLVPGG-------------------LGYDRSLAQHRQEIVDKS |
| NOV46r | ----TMVSAIVLYVLLAAAAHS-------------------AFAYDRSLAQHRQEIVDKS |
| NOV46s | GRGSTMVSAIVLYVLLAAAAHS-------------------AFAYDRSLAQHRQEIVDKS |
| NOV46t | -RGSTMVSAIVLYVLLAAAAHSAFAKPLLETLYLLGMLVPGGLGYDRSLAQHRQEIVDKS |
| NOV46a | VSPWSLETYSYNIYHPMGEIYEWMREISEKYKEVVTQHFLGVTYETHPIYYLKISQPSGN |
| NOV46b | VSPWSLETYSYNIYMPMGEIYEWMREISEKYKEVVTQHFLGVTYETHPMYYLKISQPSGN |
| NOV46c | VSPWSLETYSYNIYHPMGEIYEWMREISEKYKEVVTQHFLGVTYETHPMYYLKISQPSGN |
| NOV46d | VSPWSLETYSYNIYHPMGEIYEWMREISEKYKEVVTQHFLGVTYETHPMYYLKISQPSGN |
| NOV46e | VSPWSLETYSYNIYHPMGEIYEWMREISEKYKEVVTQHFLGVTYETHPMYYLKISQPSGN |
| NOV46f | VSPWSLETYSYNIYHPMGEIYEWMREISEKYKEVVTQHFLGVTYETHPMYYLKISQPSGN |
| NOV46g | VSPWSLETYSYNIYHPMGEIYEWMREISEKYKEVVTQHFLGVTYETHPMYYLKISQPSGN |
| NOV46h | VSPWSLETYSYNIYHPMGEIYEWMREISEKYKEVVTQHFLGVTYETHPMYYLKISQPSGN |
| NOV46i | VSPWSLETYSYNIYHPMGEINEWMREISEKYKEVVTQHFLGVTYETHPMYYLKISQPSGN |
| NOV46j | VSPWSLETYSYNIYHPMGEIYEWMREISEKYKEVVTQHFLGVTYETHPMYYLKISQPSGN |
| NOV46k | VSPWSLETYSYNIYHPMGEIYEWMREISEKYKEVVTQHFLGVTYETHPMYYLKISQPSGN |
| NOV46l | VSPWSLETYSYNIYHPMGEIYEWMREISEKYKEVVTQHFLGVTYETHPMYYLKISQPSGN |
| NOV46m | VSPWSLETYSYNIYHPMGEIYEWMREISEKYKEVVTQHFLGVTYETHPIYYLKISQPSGN |
| NOV46n | ----------------MGEIYEWMREISEKYKEVVTQHFLGVTYEThPIYYLKISQPSGN |
| NOV46o | VSPWSLETYSYNIYHPMGEIYEWMREISEKYKEVVTQHFLGVTYETHPMYYLKISQPSGN |
| NOV46p | VSPWSLETYSYNIYHPMGEIYEWMREISEKYKEVVTQHFLGVTYETHPMYYLKISQPSGN |
| NOV46q | VSPWSLETYSYNIYHPMGEIYEWMREISEKYKEVVTQHFLGVTYETHPMYYLKISQPSGN |
| NOV46r | VSPWSLETYSYNIYHPMGEIYEWMREISEKYKEVVTQHFLGVTYETHPMYYLKISQPSGN |
| NOV46s | VSPWSLETYSYNIYHPMGEIYEWMREISEKYKEVVTQHFLGVTYETHPMYYLKISQPSGN |
| NOV46t | VSPWSLETYSYNIYHPMGEIYEWMREISEKYKEVVTQHFLGVTYETHPMYYLKISQPSGN |
| NOV46a | PKKIIWMDCGIHAREWIAPAFCQWFVKEILQNHKDNSRIRKLLRNLDFYVLPVLNIDGYI |
| NOV46b | PKKIIWMDCGIHAREWIAPAFCQWFVKEILQNHKDNSRIRKLLRNLDFYVLPVLNIDGYI |
| NOV46c | PKKIIWMDCGIHAREWIAPAFCQWFVKEILQNHKDNSRIRKLLRNLDFYVLPVLNIDGYI |
| NOV46d | PKKIIWMDCGIHAREWIAPAFCQWFVKEILQNHKDNSRIRKLLRNLDFYVLPVLNIDGYI |
| NOV46e | PKKIIWMDCGIHAREWIAPAFCQWFVKEILQNHKDNSRIRKLLRNLDFYVLPVLNIDGYI |
| NOV46f | PKKIIWMDCGIHAREWIAPAFCQWFVKEILQNHKDNSRIRKLLRNLDFYVLPVLNIDGYI |
| NOV46g | PKKIIWMDCGIHAREWIAPAFCQWFVKEILQNHKDNSRIRKLLRNLDFYVLPVLNIDGYI |
| NOV46h | PKKIIWMDCGIHAREWIAPAFCQWFVKEILQNHKDNSRIRKLLRNLDFYVLPVLNIDGYI |
| NOV46i | PKKIIWMDCGIHAREWIAPAFCQWFVKEILQNHKDNSRIRKLLRNLDFYVLPVLNIDGYI |
| NOV46j | PKKIIWMDCGIHAREWIAPAFCQWFVKEILQNHKDNSRIRKLLRNLDFYVLPVLNIDGYI |
| NOV46k | PKKIIWMDCGIHAREWIAPAFCQWFVKEILQNHKDNSRIRKLLRNLDFYVLPVLNIDGYI |
| NOV46l | PKKIIWMDCGIHAREWIAPAFCQWFVKEILQNHKDNSRIRKLLRNLDFYVLPVLNIDGYI |
| NOV46m | PKKIIWMDCGIHAREWIAPAFCQWFVKEILQNHKDNSRIRKLLRNLDFYVLPVLNIDGYI |
| NOV46n | PKKIIWMDCGIHAREWIAPAFCQWFVKEILQNHKDNSRIRKLLRNLDFYVLPVLNIDGYI |
| NOV46o | PKKIIWMDCGIHAREWIAPAFCQWFVKEILQNHKDNSRIRKLLRNLDFYVLPVLNIDGYI |
| NOV46p | PKKIIWMDCGIHAREWIAPAFCQWFVKEILQNHKDNSRIRKLLRNLDFYVLPVLNIDGYI |
| NOV46q | PKKIIWMDCGIHAREWIAPAFCQWFVKEILQNHKDNSRIRKLLRNLDFYVLPVLNIDGYI |
| NOV46r | PKKIIWMDCGIHAREWIAPAFCQWFVKEILQNHKDNSRIRKLLRNLDFYVLPVLNIDGYI |
| NOV46s | PKKIIWMDCGIHAREWIAPAFCQWFVKEILQNHKDNSRIRKLLRNLDFYVLPVLNIDGYI |
| NOV46t | PKKIIWMDCGIHAREWIAPAFCQWFVKEILQNHKDNSRIRKLLRNLDFYVLPVLNIDGYI |
| NOV46a | YTWTTDRLWRKSRSPHNNGTCFGTDLNRNFNASWCSIGASRNCQDQTFCGTGPVSEPETK |
| NOV46b | YTWTTDRLWRKSRSPHNNGTCFGTDLNRNFNASWCSIGASRNCQDQTFCGTGPVSEPETK |
| NOV46c | YTWTTDRLWRKSRSPHNNGTCFGTDLNRNFNASWCSIGASRNCQDQTFCGTGPVSEPETK |
| NOV46d | YTWTTDRLWRKSRSPHNNGTCFGTDLNRNFNASWCSIGASRNCQDQTFCGTGPVSEPETK |
| NOV46e | YTWTTDRLWRKSRSPHNNGTCFGTDLNRNFNASWCSIGASRNCQDQTFCGTGPVSEPETK |
| NOV46f | YTWTTDRLWRKSRSPHNNGTCFGTDLNRNFNASWCSIGASRNCQDQTFCGTGPVSEPETK |
| NOV46g | YTWTTDRLWRKSRSPHNNGTCFGTDLNRNFNASWCSIGASRNCQDQTFCGTGPVSEPETK |
| NOV46h | YTWTTDRLWRKSRSPHNNGTCFGTDLNRNFNASWCSIGASRNCQDQTFCGTGPVSEPETK |
| NOV46i | YTWTTDRLWRKSRSPHNNGTCFGTDLNRNFNASWCSIGASRNCQDQTFCGTGPVSEPETK |
| NOV46j | YTWTTDRLWRKSRSPHNNGTCFGTDLNRNFNASWCSIGASRNCQDQTFCGTGPVSEPETK |
| NOV46k | YTWTTDRLWRKSRSPHNNGTCFGTDLNRNFNASWCSIGASRNCQDQTFCGTGPVSEPETK |
| NOV46l | YTWTTDRLWRKSRSPHNNGTCFGTDLNRNFNASWCSIGASRNCQDQTFCGTGPVSEPETK |
| NOV46m | YTWTTDRLWRKSRSPHNNGTCFGTDLNRNFNASWCNSSWTEGSKCIESKVWNQL------ |
| NOV46n | YTWTTDRLWRKSRSPHNNGTCFGTDLNRNFNASWCSIGASRNCQDQTFCGTGPVSEPETK |
| NOV46o | YTWTTDRLWRKSRSPHNNGTCFGTDLNRNFNASWCSIGASRNCQDQTFCGTGPVSEPETK |
| NOV46p | YTWTTDRLWRKSRSPHNNGTCFGTDLNRNFNASWCSIGASRNCQDQTFCGTGPVSEPETK |
| NOV46q | YTWTTDRLWRKSRSPHNNGTCFGTDLNRNFNASWCSIGASRNCQDQTFCGTGPVSEPETK |
| NOV46r | YTWTTDRLWRKSRSPHNNGTCFGTDLNRNFNASWCSIGASRNCQDQTFCGTGPVSEPETK |
| NOV46s | YTWTTDRLWRKSRSPHNNGTCFGTDLNRNFNASWCSIGASRNCQDQTFCGTGPVSEPETK |
| NOV46t | YTWTTDRLWRKSRSPHNNGTCFGTDLNRNFNASWCSIGASRNCQDQTFCGTGPVSEPETK |
| NOV46a | AVASFIESKKDDILCFLTMHSYGQLILTPYGYTKNKSSNHPEMIQVGQKAANALKAKYGT |
| NOV46b | AVASFIESKKDDILCFLTMHSYGQLILTPYGYTKNKSSNHPEMIQVGQKAANALKAKYGT |
| NOV46c | AVASFIESKKDDILCFLTMHSYGQLILTPYGYTKNKSSNHPEMIQVGQKAANALKAKYGT |
| NOV46d | AVASFIESKKDDILCFLTMHSYGQLILTPYGYTKNKSSNHPEMIQVGQKAANALKAKYGT |
| NOV46e | AVASFIESKKDDILCFLTMHSYGQLILTPYGYTKNKSSNHPEMIQVGQKAANALKAKYGT |
| NOV46f | AVASFIESKKDDILCFLTMHSYGQLILTPYGYTKNKSSNHPEMIQVGQKAANALKAKYGT |
| NOV46g | AVASFIESKKDDILCFLTMHSYGQLILTPYGYTKNKSSNHPEMIQVGQKAANALKAKYGT |
| NOV46h | AVASFIESKKDDILCFLTMHSYGQLILTPYGYTKNKSSNHPEMIQVGQKAANALKAKYGT |
| NOV46i | AVASFIESKKDDILCFLTMHSYGQLILTPYGYTKNKSSNHPEMIQVGQKAANALKAKYGT |
| NOV46j | AVASFIESKKDDILCFLTMHSYGQLILTPYGYTKNKSSNHPEMIQVGQKAANALKAKYGT |
| NOV46k | AVASFIESKKDDILCFLTMHSYGQLILTPYGYTKNKSSNHPEMIQVGQKAANALKAKYGT |
| NOV46l | AVASFIESKKDDILCFLTMHSYGQLILTPYGYTKNKSSNHPEMIQVGQKAANALKAKYGT |
| NOV46m | ------------------------------------------------------------ |
| NOV46n | AVASFIESKKDDILCFLTMHSYGQLILTPYGYTKNKSSNHPEMIQVGQKAANALKAKYGT |
| NOV46o | AVASFIESKKDDILCFLTMHSYGQLILTPYGYTKNKSSNHPEMIQVGQKAANALKAKYGT |
| NOV46p | AVASFIESKKDDILCFLTMHSYGQLILTPYGYTKNKSSNHPEMIQVGQKAANALKAKYGT |
| NOV46q | AVASFIESKKDDILCFLTMHSYGQLILTPYGYTKNKSSNHPEMIQVGQKAANALKAKYGT |
| NOV46r | AVASFIESKKDDILCFLTMHSYGQLILTPYGYTKNKSSNHPEMIQVGQKAANALKAKYGT |
| NOV46s | AVASFIESKKDDILCFLTMHSYGQLILTPYGYTKNKSSNHPEMIQVGQKAANALKAKYGT |
| NOV46t | AVASFIESKKDDILCFLTMHSYGQLILTPYGYTKNKSSNHPEMIQVGQKAANALKAKYGT |
| NOV46a | NYRVGSSADILYASSGSSRDWARDIGIPFSYTFELRDSGTYGFVLPEAQIQPTCEETMEA |
| NOV46b | NYRVGSSADILYASSGSSRDWARDIGIPFSYTFELRDSGTYGFVLPEAQIQPTCEETMEA |
| NOV46c | NYRVGSSADILYASSGSSRDWARDIGIPFSYTFELRDSGTYGFVLPEAQIQPTCEETMEA |
| NOV46d | NYRVGSSADILYASSGSSRDWARDIGIPFSYTFELRDSGTYGFVLPEAQIQPTCEETMEA |
| NOV46e | NYRVGSSADILYASSGSSRDWARDIGIPFSYTFELRDSGTYGFVLPEAQIQPTCEETMEA |
| NOV46f | NYRVGSSADILYASSGSSRDWARDIGIPFSYTFELRDSGTYGFVLPEAQIQPTCEETMEA |
| NOV46g | NYRVGSSADILYASSGSSRDWARDIGIPFSYTFELRDSGTYGFVLPEAQIQPTCEETMEA |
| NOV46h | NYRVGSSADILYASSGSSRDWARDIGIPFSYTFELRDSGTYGFVLPEAQIQPTCEETMEA |
| NOV46i | NYRVGSSADILYASSGSSRDWARDIGIPFSYTFELRDSGTYGFVLPEAQIQPTCEETMEA |
| NOV46j | NYRVGSSADILYASSGSSRDWARDIGIPFSYTFELRDSGTYGFVLPEAQIQPTCEETMEA |
| NOV46k | NYRVGSSADILYASSGSSRDWARDIGIPFSYTFELRDSGTYGFVLPEAQIQPTCEETMEA |
| NOV46l | NYRVGSSADILYASSGSSRDWARDIGIPFSYTFELRDSGTYGFVLPEAQIQPTCEETMEA |
| NOV46m | ------------------------------------------------------------ |
| NOV46n | NYRVGSSADILYASSGSSRDWARDIGIPFSYTFELRDSGTYGFVLPEAQIQPTCEETMEA |
| NOV4Go | NYRVGSSADILYASSGSSRDWARDIGIPFSYTFELRDSGTYGFVLPEAQIQPTCEETMEA |
| NOV46p | NYRVGSSADILYASSGSSRDWARDIGIPFSYTFELRDSGTYGFVLPEAQIQPTCEETMEA |
| NOV46q | NYRVGSSADILYASSGSSRDWARDIGIPFSYTFELRDSGTYGFVLPEAQIQPTCEETMEA |
| NOV46r | NYRVGSSADILYASSGSSRDWARDIGIPFSYTFELRDSGTYGFVLPEAQIQPTCEETMEA |
| NOV46s | NYRVGSSADILYASSGSSRDWARDIGIPFSYTFELRDSGTYGFVLPEAQIQPTCEETMEA |
| NOV46t | NYRVGSSADILYASSGSSRDWARDIGIPFSYTFELRDSGTYGFVLPEAQIQPTCEETMEA |
| NOV46a | VLSVLDDVYAKHWHSDSAGRVTSATMLLGLLVSCMSLL------ |
| NOV46b | VLSVLDDVYAKHWHSDSAGRVTSATMLLGLLVSCMSLL------ |
| NOV46c | VLSVLDDVYAKHWHSDSAGRVTSATMLLGLLVSCMSLLHHHHHH |
| NOV46d | VLSVLDDVYAKHWHSDSAGRVTSATMLLGLLVSCMSLL------ |
| NOV46e | VLSVLDDVYAKHWHSDSAGRVTSATMLLGLLVSCMSLLHHHHHH |
| NOV46f | VLSVLDDVYAKHWHSDSAGRVTSATMLLGLLVSCMSLL------ |
| NOV46g | VLSVLDDVYAKHWHSDSAGRVTSATMLLGLLVSCMSLL------ |
| NOV46h | VLSVLDDVYAKHWHSDSAGRVTSATMLLGLLVSCMSLL------ |
| NOV46i | VLSVLDDVYAKHWHSDSAGRVTSATMLLGLLVSCMSLL------ |
| NOV46j | VLSVLDDVYAKHWHSDSAGR------------------------ |
| NOV46k | VLSVLDDVYAKHWHSDSAGRVTSATMLLGLLVSCMSLL------ |
| NOV46l | VLSVLDDVYAKHWHSDSAGRVTSATMLLGLLVSCMSLL------ |
| NOV46m | -------------------------------------------- |
| NOV46n | VLSVLDDVYAKHWHSDSAGRVTSATMLLGLLVSCMSLL------ |
| NOV46o | VLSVLDDVYAKHWHSDSAGRVTSATMLLGLLVSCMSLL------ |
| NOV46p | VLSVLDDVYAKHWHSDSAGRVTSATMLLGLLVSCMSLL------ |
| NOV46q | VLSVLDDVYAKHWHSDSAGRVTSATMLLGLLVSCMSLLHHHHHH |
| NOV46r | VLSVLDDVYAKHWHSDSAGRVTSATMLLGLLVSCMSLLHHHHHH |
| NOV46s | VLSVLDDVYAKHWHSDSAGRVTSATMLLGLLVSCMSLL------ |
| NOV46t | VLSVLDDVYAKHWHSDSAGRVTSATMLLGLLVSCMSLLHHHHHH |
| NOV46a | (SEQ ID NO: 700) |
| NOV46b | (SEQ ID NO: 702) |
| NOV46c | (SEQ ID NO: 704) |
| NOV46d | (SEQ ID NO: 706) |
| NOV46e | (SEQ ID NO: 708) |
| NOV46f | (SEQ ID NO: 710) |
| NOV46g | (SEQ ID NO: 712) |
| NOV46h | (SEQ ID NO: 714) |
| NOV46i | (SEQ ID NO: 716) |
| NOV46j | (SEQ ID NO: 718) |
| NOV46k | (SEQ ID NO: 720) |
| NOV46l | (SEQ ID NO: 722) |
| NOV46m | (SEQ ID NO: 724) |
| NOV46n | (SEQ ID NO: 726) |
| NOV46o | (SEQ ID NO: 728) |
| NOV46p | (SEQ ID NO: 730) |
| NOV46q | (SEQ ID NO: 732) |
| NOV46r | (SEQ ID NO: 734) |
| NOV46s | (SEQ ID NO: 736) |
| NOV46t | (SEQ ID NO: 738) |

Further analysis of the NOV46a protein yielded the following properties shown in Table 46C.

TABLE 46C
Protein Sequence Properties NOV46a

| | |
|---|---|
| SignalP analysis: | Cleavage site between residues 21 and 22 |
| PSORT II analysis: | |

| PSG: | a new signal peptide prediction method | | | |
|---|---|---|---|---|
| | N-region: | length 6; | pos.chg 1; | neg.chg 1 |
| | H-region: | length 15; | peak value | 9.15 |

| | PSG score: 4.75 |
|---|---|
| GvH: | von Heijne's method for signal seq. recognition |
| | GvH score (threshold: −2.1): −0.52 |
| | possible cleavage site: between 20 and 21 |

>>> Seems to have a cleavable signal peptide (1 to 20)

| ALOM: | Klein et al's method for TM region allocation | | |
|---|---|---|---|
| | Init position for calculation: 21 | | |
| | Tentative number of TMS(s) for the threshold 0.5: 1 | | |
| | Number of TMS(s) for threshold 0.5: 1 | | |
| | INTEGRAL | Likelihood = −2.34 | Transmembrane 357-373 |

| | |
|---|---|
| PERIPHERAL | Likelihood = 4.08 (at 229) |

ALOM score: −2.34 (number of TMSs: 1)

MTOP: Prediction of membrane topology (Hartmann et al.)

Center position for calculation: 10
Charge difference: −0.5 C(0.5) − N(1.0)
N >= C: N-terminal side will be inside

>>> membrane topology: type 1a (cytoplasmic tail 374 to 374)
>>> Seems to be GPI anchored
MITDISC: discrimination of mitochondrial targeting seq

| | | | |
|---|---|---|---|
| R content: | 0 | Hyd Moment(75): | 8.63 |
| Hyd Moment(95): | 12.49 | G content: | 4 |
| D/E content: | 2 | S/T content: | 1 |
| Score: −7.55 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 9.9%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

55.6%: extracellular, including cell wall
22.2%: endoplasmic reticulum
11.1%: Golgi
11.1%: plasma membrane

>> prediction for CG55794-03 is exc (k = 9)

A search of the NOV46a protein against the Geneseq database, a proprietary database that contains sequences published in patents and patent publication, yielded several homologous proteins shown in Table 46D.

TABLE 46D
Geneseq Results for NOV46a

| | | NOV46a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66547 | Human secreted | 1 . . . 374 | 374/374 (100%) | 0.0 |
| | metallocarboxypeptidase-like | 1 . . . 374 | 374/374 (100%) | |
| | polypeptide - Homo sapiens, 374 | | | |
| | aa. [WO200157265-A1, 09- | | | |
| | AUG-2001] | | | |
| AAG66565 | Human secreted | 1 . . . 374 | 373/374 (99%) | 0.0 |
| | metallocarboxypeptidase-like | 1 . . . 374 | 374/374 (99%) | |
| | variant polypeptide - Homo | | | |
| | sapiens, 374 aa. [WO200157265- | | | |
| | A1, 09-AUG-2001] | | | |
| AAU82703 | Amino acid sequence of novel | 1 . . . 374 | 372/374 (99%) | 0.0 |
| | human protease #2 - Homo | 1 . . . 374 | 373/374 (99%) | |
| | sapiens, 374 aa. [WO200200860- | | | |
| | A2, 03-JAN-2002] | | | |
| AAB74682 | Human protease and protease | 13 . . . 374 | 360/362 (99%) | 0.0 |
| | inhibitor PPIM-15 - Homo | 1 . . . 362 | 361/362 (99%) | |
| | sapiens, 362 aa. [WO200110903- | | | |
| | A2, 15-FEB-2001] | | | |
| AAG66560 | Human secreted | 21 . . . 374 | 354/354 (100%) | 0.0 |
| | metallocarboxypeptidase-like | 1 . . . 354 | 354/354 (100%) | |
| | polypeptide - Homo sapiens, 354 | | | |
| | aa. [WO200157265-A1, 09- | | | |
| | AUG-2001] | | | |

In a BLAST search of public sequence databases, the NOV46a protein was found to have homology to the proteins shown in the BLASTP data in Table 46E.

TABLE 46E
Public BLASTP Results for NOV46a

| | | NOV46a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IVL8 | Zn-carboxypeptidase - Homo | 1 . . . 374 | 372/374 (99%) | 0.0 |
| | sapiens (Human), 374 aa. | 1 . . . 374 | 373/374 (99%) | |
| Q9PUF2 | Carboxypeptidase homolog - | 13 . . . 337 | 152/326 (46%) | 7e−87 |
| | Bothrops jararaca (Jararaca), 416 | 82 . . . 405 | 219/326 (66%) | |
| | aa. | | | |
| Q8AXN3 | Carboxypeptidase B - | 41 . . . 337 | 146/297 (49%) | 8e−86 |
| | Paralichthys olivaceus (Flounder), | 100 . . . 396 | 203/297 (68%) | |
| | 408 aa (fragment). | | | |
| CAA03381 | SEQUENCE 85 FROM PATENT | 45 . . . 352 | 148/312 (47%) | 1e−85 |
| | WO9620011 - unidentified, 349 | 26 . . . 336 | 204/312 (64%) | |
| | aa. | | | |
| Q9JHH6 | Carboxypeptidase R (Thrombin- | 30 . . . 350 | 145/323 (44%) | 2e−85 |
| | activatable fibrinolysis inhibitor) | 103 . . . 417 | 210/323 (64%) | |
| | (1110032P04Rik protein) - Mus | | | |
| | musculus (Mouse), 422 aa. | | | |

PFam analysis indicates that the NOV46a protein contains the domains shown in the Table 46F.

TABLE 46F
Domain Analysis of NOV46a

| | | Identities/ | |
|---|---|---|---|
| | NOV46a | Similarities for | |
| Pfam Domain | Match Region | the Matched Region | Expect Value |
| Zn_carbOpept | 48..249 | 82/215 (38%) | 1.2e-74 |
| | | 168/215 (78%) | |

### Example 47

The NOV47 clone was analyzed, and the nucleotide and encoded polypeptide sequences are shown in Table 47A.

TABLE 47A

NOV47 Sequence Analysis
NOV47a,

| | | |
|---|---|---|
| CG55806-04 | SEQ ID NO: 747 | 1300 bp |
| DNA Sequence | ORF Start: ATG at 20 | ORF Stop: TGA at 1274 |

TGACTGTATCGCCGGATTCATGCAGCGCGTGAACATGATCATGGCAGAATCACCAGGCCTCATCACCA
TCTGCCTTTTAGGATATCTACTCAGTGCTGAATGTACAGTTTTTCTTGATCATGAAAACGCCAACAAA
ATTCTGAATCGGCCAAAGAGGTATAATTCAGGTAAATTGGAGGAGTTTGTTCATGGGAACCTTGAGAG
AGAATGTATGGAAGAAAAGTGCAGTTTTGAAGAAGCACGAGAAGTTTTTGAAAACACTGAAAGAACAA
CTGAATTTTGGAAGCAGTATGTTGATGGAGATCAGTGTGAGTCCAATCCATGTTTAAATGGCGGCAGT
TGCAAGGATGTCATTAATTCCTATGAATGTTGGTGTCCCTTTGGATTTGAAGGAAAGAACTGTGAATT
AGTGCCATTTCCATGTGGAAGAGTTTCTGTTTCACAAACTTCTAAGCTCACCCGTGCTGAGACTGTTT
TTCCTGATGTGGACTATGTAAATTCTACTGAAGCTGAAACCATTTTGGATAACATCACTCAAAGCACC
CAATCATTTAATGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAAATGGATTGTAA
CTGCTGCCCACTGTGTTGAAACTGGTGTTAAAATTACAGTTGTCGCAGGTGAACATAATATTGAGGAG
ACAGAACATACAGAGCAAAAGCGAAATGTGATTCGAATTATTCCTCACCACAACTACAATGCAGCTAT
TAATAAGTACAACCATGACATTGCCCTTCTGGAACTGGACGAACCCTTAGTGCTAAACAGCTACGTTA
CACCTATTTGCATTGCTGACAAGGAATACACGAACATCTTCCTCAAATTTGGATCTGGCTATGTAAGT
GGCTGGGGAAGAGTCTTCCACAAAGGGAGATCAGCTTTAGTTCTTCAGTACCTTAGAGTTCCACTTGT
TGACCGAGCCACATGTCTTGATCTACAAAGTTCACCATCTATAACAACATGTTCTGTGCTAGGCTTCC
ATGAAGGAGGTAGAGATTCATGTCAAGGAGATAGTGGGGGACCCCATGTTACTGAAGTGGAAGGGACC
AGTTTCTTAACTGGAATTATTAGCTGGGGTGAAGAGTGTGCAATGAAAGGCAAATATGGAATATATAC
CAAGGTATCCCGGTATGTCAACTGGATTAAGGAAAAAACAAAGCTCACTTGACTGCAGCCAAGCTAAT
TCCGGAAG

| | | | |
|---|---|---|---|
| NOV47a, | | | |
| GG55806-04 | | | |
| Protein Sequence | SEQ ID NO: 748 | 418 aa | MW at 47031.8kD |

MQRVNMIMAESPGLITICLLGYLLSAECTVFLDHENANKILNRPKRYNSGKLEEFVHGNLERECMEEK
CSFEEAREVFENTERTTEFWKQYVDGDQCESNPCLNGGSCKDVINSYECWCPFGFEGKNCELVPFPCG
RVSVSQTSKLTRAETVFPDVDYVNSTEAETILDNITQSTQSFNDFTRVVGGEDAKPGQFPWQVVLNGK
VDAFCGGSIVNEKWIVTAAHCVETGVKITVVAGEHNIEETEHTEQKRIVIRIIPHNNYNAAINKYNHD
IALLELDEPLVLNSYVTPICIADKEYLIIFLKFGSGYVSGWGRVFHKGRSALVLQYLRVPLVDRATCL
RSTKFTIYNNMFCAGFHEGGRDSCQGDSGGPHVTEVEGTSFLTGIISWGEECAMKGKYGIYTKVSRYV
NWIKEKTKLT

| | | |
|---|---|---|
| NOV47b, | | |
| CG55806-01 | SEQ ID NO: 749 | 1438 bp |
| DNA Sequence | ORF Start: ATG at 2 | ORF Stop: TAA at 1184 |

TATGCAGCGCGTGAACATGATCATGGCAGAATCACCAGGCCTCATCACCATCTGCCTTTTAGGATATC
TACTCAGTGCTGAATGTACAGTTTTTCTTGATCATGAAAACGCCAACAAAATTCTGAATCGGCCAAAG
AGGTATAATTCAGGTAAATTGGAAGAGTTTGTTCAAGGGGAACCTTGAGAGAGAATGTCTGGAGGAAA
GTGTAGTTTTGAAGAAGCACGAGAAGTTTTTGAAAACACTGAAAGAACAACTGAATTTTGGAAGCAGT
ATGTTGATGGAGATCAGTGTGAGTCCAATCCATGTTTAAATGGCGGCAGTTGCAAGGATGACATTAAT
TCCTATGAATGTTGGTGTCCCTTTGGATTTGAAGGAAAGAACTGTGAATTAGATGTGGACTATGTAAA
TTCTACTGAAGCTGAAACCATTTTGGATAACATCACTCAAAGCACCCAATCATTTAATGACTTCACTC
GGGTTGTTGGTGGAGAAGATGCCAAACCAGGTCAATTCCCTTGGCAGGTTGTTTTGAATGGTAAAGTT
GATGCATTCTGTGGAGGCTCTATCGTTAATGAAAAATGGATTGTAACTGCTGCCCACTGTGTTGAAAC
TGGTGTTAAAATTACAGTTGTCGCAGGTGAACATAATATTGAGGAGACAGAACATACAGAGCAAAAGC
GAAATGTGATTCGAATTATTCCTCACCACAACTACAATGCAGCTATTAATAAGTACAACCATGACATT
GCCCTTCTGGAACTGGACGAACCCTTAGTGCTAAACAGCTACGTTACACCTATTTGCATTGCTGACAA
GGAATACACGAACATCTTCCTCAAATTTGGATCTGGCTATGTAAGTGGCTGGGGAAGAGTCTTCCACA
AAGGGAGATCAGCTTTAGTTCTTCAGTACCTTAGAGTTCCACTTGTTGACCGAGCCACATGTCTTCGA
TCTACAAAGTTCACCATCTATAACAACATGTTCTGTGCTGGCTTCCATGAAGGAGGTAGAGATTCATG
TCAAGGAGATAGTGGGGGACCCCATGTTACTGAAGTGGAAGGGACCAGTTTCTTAACTGGAATTATTA
GCTGGGGTGAAGAGTGTGCAATGAAAGGCAAATATGGAATATATACCAAGGTATCCCGGTATGTCAAC
TGGATTAAGGAAAAAACAAAGCTCACTTAATGAAAGATGGATTTCCAAGGTTAATTCATTGGAATTGA
AAATTAACAGGGCCTCTCACTAACTAATCACTTTCCCATCTTTTGTTAGATTTGAATATATACATTCT
ATGATCATTGCTTTTTCTCTTTACAGGGGAGAATTTCATATTTTACCTGAGCAAATTGATTAGAAAAT
GGAACCACTAGAGGAATATAATGTGTTAGGAAATTACAGTCATTTCTAAGGGCCCAGCCTTGACAAAT
TGTGAGTAAA

| | | | |
|---|---|---|---|
| NOV47B, CG55806-01 | | | |
| Protein Sequence | SEQ ID NO: 750 | 394 aa | MW at 44431.7kD |

MQRVNMIMAESPGLITICLLGYLLSAECTVFLDHENANKILNRPKRYNSGKLEEFVQGNLERECLEEK
CSFEEAREVFENTERTTEFWKQYVDGDQCESNPCLNGGSCKDDINSYECWCPFGFEGKNCELDVDYVN
STEAETILDNITQSTQSFNDFTRVVGGEDAKPGQFPWQVVLNGKVDAFCGGSIVNEKWIVTAAHCVET
GVKITVVAGEHNIEETEHTEQKRNVIRIIPHHNYNAAINKYNHDIALLELDEPLVLNSYVTPICIADK
EYTNIFLKFGSGYVSGWGRVFHKGRSALVLQYLRVPLVDRATCLRSTKFTIYNNMFCAGFHEGGRDSC
QGDSGGPHVTEVEGTSFLTGIISWGEECAMKGKYGIYTKVSRYVNWIKEKTKLT

| | | |
|---|---|---|
| NOV47c, CG55806-02 | SEQ ID NO: 751 | 2807 bp |
| DNA Sequence | ORF Start: ATG at 20 | ORF Stop: TAA at 1403 |

CAATCTGCTAGCAAAGGTTATGCAGCGCGTGAACATGATCATGGCAGAATCACCAGGCCTGATCACCA
TCTGCCTTTTAGGATATCTACTCAGTGCTGAATGTACAGTTTTTCTTGATCATGAAAACGCCAACAAA
ATTCTGAATCGGCCAAAGAGGTATAATTCAGGTAAATTGGAAGAGTTTGTTCAAGGGAACCTTGAGAG
AGAATGTATGGAAGAAAAGTGTAGTTTTGAAGAAGCACGAGAAGTTTTTGAAAACACTGAAAGAACAA
CTGAATTTTGGAAGCAGTATGTTGATGGAGATCAGTGTGAGTCCAATCCATGTTTAAATGGCGGCAGT
TGCAAGGATGACATTAATTCCTATGAATGTTGGTGTCCCTTTGGATTTGAAGGAAAGAACTGTGAATT
AGATGTAACATGTAACATTAAGAATGGCAGATGAGCAGTTTTGTAAAAATAGTGCTGATAACACAAGG
TGGTTTGCTCCTGTACTGAGGGATATCGACTTGCAGAAAACCAGAAGTCCTGTGAACCAGCAGTGCCA
TTTCCATGTGGAAGAGTTTCTGTTTCACAAACTTCTAAGCTCACCCGTGCTGAGACTGTTTTTCCTGA
TGTGGACTATGTAAATTCTACTGAAGCTGAAACCATTTTGGATAACATCACTCAAAGCACCCAATCAT
TTAATGACTTCACTCGGGTTGTTGGTGGAGAAGATGCCAAACCAGGTCAATTCCCTTGGCAGGTTGTT
TTGAATGGTAAAGTTGATGCATTCTGTGGAGGCTCTATCGTTAATGAAAAATGGATTGTAACTGCTGC
CCACTGTGTTGAAACTGGTGTTAAAATTACAGTTGTCGCAGGTGAACATAATATTGAGGAGACAGAAC
ATACAGAGCAAAGCGAAATGTGATTCGAATTATTCCTCACCACAACTACAATGCAGCTAATTAATAAG
TACAACCATGCATTGCCCTTCTGGAACTGGACGAACCCTTAGTGCTAAACAGCTACGTTACAACCTAT
TTGCATTGCTGACAAGGAATACACGAACATCTTCCTCAAATTTGGATCTGGCTATGTAAGTGGCTGGG
GAAGAGTCTTCCACAAAGGGAGATCAGCTTTAGTTCTTCAGTACCTTAGAGTTCCACTTGTTGACCGA
GCCACATGTCTTCGATCTACAAAGTTCACCATCTATAACAACATGTTCTGTGCTGGCTTCCATGAAGG
AGGTAGAGATTCATGTCAAGGAGATAGTGGGGGACCCCATGTTACTGAAGTGGAAGGGACCAGTTTCT
TAACTGGAATTATTAGCTGGGGTGAAGAGTGTGCAATGAAAGGCAAATATGGAATATATACCAAGGTA
TCCCGGTATGTCAACTGGATTAAGGAAAAAACAAAGCTCACTTAATGAAAGATGGATTTCCAAGGTTA
ATTCATTGGAATTGAAAATTAACAGGGCCTCTCACTAACTAATCACTTTCCCATCTTTTGTTAGATTT
GAATATATACATTCTATGATCATTGCTTTTTCTCTTTACAGGGGAGAATTTCATATTTTACCTGAGCA
AATTGATTAGAAAATGGAACCACTAGAGGAATATAATGTGTTAGGAAATTACAGTCATTTCTGAAGCC
CAGCCCTTGACAAAATTGTGAAGTTAAATTCTCCACTCTGTCCATCAGATACTATGGTTCTCCACTAT
GGCAACTAACTCACTCAATTTTCCCTCCTTAGCAGCATTCCATCTTCCCGATCTTCTTTGCTTCTCCA
ACCAAAACATCATGTTTATTAGTTCTGTATACAGTACAGGATCTTTGGTCTACTCTATCACGAGAAGG
CTCAGTACCACACTCATGAAGAAAGAACACAGGAGTAGCTGAGAGGCTAAAACTCATCAAAAACACTA
CTCCTTTTCCTCTACCCTATTCCTCAATCTTTTACCTTTTCCAAATTCCCAATTCCCCAAATCAGTTT
TTCTCTTTCTTACTCCCTCTCTCCCTTTTACCCTCCATGGTCGTTAAAGGAGAGATGGGGAGCATCAT
TCTGTTATACTTCTGTACACAGTTATACATGTCTATCAAACCCAGACTTGCTTCCATAGTGGAGACTT
GCTTTTCAGAACATAGGGATGAAGTAAGGTGCCTGAAAAGTTTGGGGGAAAAGTTTCTTTCAGAGAGT
TAAGTTATTTTATATATATAATATATATATAAAATATATAATATACAATATAAATATATAGTGTGTGT
GTGTATGCGTGTGTGTAGACACACACGCATACACACATATAATGGAAGCAATAAGCCATTCTTAAGAG
CTTGTATGGTTATGCAGGTCTGACTAGGCATGATTTCACGAAGGCAAGATTGGCATATCAGTTGTAAC
TAAAAAAGCTGACATTGACCCAGACATATTGTACTCTTTCTAAAAATAATAATAATAATGCTAACAGA
AAGAAGAGAACCGTTCGTTTGCAATCTACAGCTAGTAGAGACTTGAGGAAGAATTCAACAGTGTGTCT
TCAGCAGTGTTCAGAGCCAAGCAAGAAGTTGAAGTTGCCTAGACCAGAGGACATAAGTATCATGTCTC
CTTTAACTAGCATACCCCGAAGTGGAGAAGGATGCAGCAGGCTCAAAGGCATAAGTCATTCCAATCAG
CCAACTAAGTTGTCCTTTTCTGGTTTCGTGTTCACCATGGAACATTTTGATTATAGTTAATCCTTCTA
TCTTGAATCTTCTAGAGAGTTGCTGACCAACTGACGTATGTTTCCCTTTGTGAATTAATAAACTGGTG
TTCTGGTTCAAAAAAAAAA

| | | | |
|---|---|---|---|
| NOV47c, CG55806-02 | | | |
| Protein Sequence | SEQ ID NO: 752 | 461 aa | MW at 51778.0kD |

MQRVNMIMAESPGLITICLLGYLLSAECTVFLDHENANKILNRPKRYNSGKLEEFVQGNLERECMEEK
CSFEEAREVFENTERTTEFWKQYVDGDQCESNPCLNGGSCKDDINSYECWCPFGFEGKNCELDVTCNI
KNGRCEQFCKNSADNKVVCSCTEGYRLAENQKSCEPAVPFPCGRVSVSQTSKLTRAETVFPDVDYVNS
TEAETILDNITQSTQSFNDFTRVVGGEDAKPGQFPWQVVLNGKVDAFCGGSIVNEKWIVTAAHCVETG
VKITVVAGEHNIEETEHTEQKRNVIRIIPHHNYNAAINKYNHDIALLELDEPLVLNSYVTPICIADKE
YTNIFLKFGSGYVSGWGRVFHKGRSALVLQYLRVPLVDRATCLRSTKFTIYNNMFCAGFHEGGRDSCQ
GDSGGPHVTEVEGTSFLTGIISWGEECAMKGKYGIYTKVSRYVNWIKEKTKLT

| | | |
|---|---|---|
| NOV47d, CG55806-03 | SEQ ID NO: 753 | 1612 bp |
| DNA Sequence | ORF Start: ATG at 22 | ORF Stop: TAA at 1405 |

TTGACTGTATCGCCGGAATTCATGCAGCGCGTGAACATGATCATGGCAGAATCACCAGGCCTCATCAC
CATCTGCCTTTTAGGATATCTACTCAGTGCTGAATGTACAGTTTTTCTTGATCATGAAAACGCCAACA
AAATTCTGAATCGGCCAAAGAGGTATAATTCAGGTAAATTGGAAGAGTTTGTTCAAGGGAACCTTGAG
AGAGAATGTATGGAAGAAAAGTGTAGTTTTGAAGAAGCACGAGAAGTTTTTGAAAACACTGAAAGAAC
AACTGAATTTTGGAAGCAGTATGTTGATGGAGATCAGTGTGAGTCCAATCCATGTTTAAATGGCGGCA
GTTGCAAGGATGACATTAATTCCTATGAATGTTGGTGTCCCTTTGGATTTGAAGGAAAGAACTGTGAA
TTAGATGTAACATGTAACATTAAGAATGGCAGATGCGAGCAGTTTTGTAAAAATAGTGCTGATAACAA
GGTGGTTTGCTCCTGTACTGAGGGATATCGACTTGCAGAAAACCAGAAGTCCTGTGAACCAGCAGTGC
CATTTCCATGTGGAAGAGTTTCTGTTTCACAAACTTCTAAGCTCACCCGTGCTGAGACTGTTTTTCCT
GATGTGGACTATGTAAATTCTACTGAAGCTGAAACCATTTTGGATAACATCACTCAAAGCACCCAATC
ATTTAATGACTTCACTCGGGTTGTTGGTGGAGAAGATGCCAAACCAGGTCAATTCCCTTGGCAGGTTG
TTTTGAATGGTAAAGTTGATGCATTCTGTGGAGGCTCTATCGTTAATGAAAAATGGATTGTAACTGCT
GCCCACTGTGTTGAAACTGGTGTTAAAATTACAGTTGTCGCAGGTGAACATAATATTGAGGAGACAGA
ACATACAGAGCAAAAGCGAAATGTGATTCGAATTATTCCTCACCACAACTACAATGCAGCTATTAATA
AGTACAACCATGACATTGCCCTTCTGGAACTGGACGAACCCTTAGTGCTAAACAGCTACGTTACACCT
ATTTGCATTGCTGACAAGGAATACACGAACATCTTCCTCAAATTTGGATCTGGCTATGTAAGTGGCTG
GGGAAGAGTCTTCCACAAAGGGAGATCAGCTTTAGTTCTTCAGTACCTTAGAGTTCCACTTGTTGACC
GAGCCACATGTCTTCGATCTACAAAGTTCACCATCTATAACAACATGTTCTGTGCTGGCTTCCATGAA
GGAGGTAGAGATTCATGTCAAGGAGATAGTGGGGGACCCCATGTTACTGAAGTGGAAGGGACCAGTTT
CTTAACTGGAATTATTAGCTGGGGTGAAGAGTGTGCAATGAAAGGCAAATATGGAATATATACCAAGG
TATCCCGGTATGTCAACTGGATTAAGGAAAAAACAAAGCTCACTTAATGAAAGATGGATTTCCAAGGT
TAATTCATTGAAATTGAAAATTAATAGGGCCTCTCACTAACTAATCACTTTCCCATCTTTTGTTAGAT
TTGAATATATACATTCTATGATCATTGCTTTTTCTCTTTACAGGGGAGAATTTCATATTTTACCTGAG
CAAATTGATTAGAAAATGGAACCACTAGAGGAATATAATGTGTTAGGA

| | | | |
|---|---|---|---|
| NOV47d, CG55806-03 | | | |
| Protein Sequence | SEQ ID NO: 754 | 461 aa | MW at 51778.0kD |

MQRVNNIMAESPGLITICLLGYLLSAECTVFLDHENANKILNRPKRYNSGKLEEFVQGNLERECMEEK
CSFEEAREVFENTERTTEFWKQYVDGDQCESNPCLNGGSCKDDINSYECWCPFGFEGKNCELDVTCNI
KNGRCEQFCKNSADNKVVCSCTEGYRLAENQKSCEPAVPFPCGRVSVSQTSKLTRAETVFPDVDYVNS
TEAETILDNITQSTQSFNDFTRVVGGEDAKPGQFPWQVVLNGKVDAFCGGSIVNEKWIVTAAHCVETG
VKITVVAGEHNIEETEHTEQKRNVIRIIPHHNYNAAINKYNHDIALLELDEPLVLNSYVTPICIADKE
YTNIFLKFGSGYVSGWGRVFHKGRSALVLQYLRVPLVDRATCLRSTKFTIYNNMFCAGFHEGGRDSCQ
GDSGGPHVTEVEGTSFLTGIISWGEECAMKGKYGIYTKVSRYVNWIKEKTKLT

| | | |
|---|---|---|
| NOV47e, | | |
| SNP13382503 of | SEQ ID NO: 755 | 1300 bp |
| CG55806-04, | ORF Start: ATG at 20 | ORF Stop: TGA at 1274 |
| DNA Sequence | SNP Pos: 470 | SNP Change: A to G |

TGACTGTATCGCCGGATTCATGCAGCGCGTGAACATGATCATGGCAGAATCACCAGGCCTCATCACCA
TCTGCCTTTTAGGATATCTACTCAGTGCTGAATGTACAGTTTTTCTTGATCATGAAAACGCCAACAAA
ATTCTGAATCGGCCAAAGAGGTATAATTCAGGTAAATTGGAGGAGTTTGTTCATGGGAACCTTGAGAG
AGAATGTATGGAAGAAAAGTGCAGTTTTGAAGAAGCACGAGAAGTTTTTGAAAACACTGAAAGAACAA
CTGAATTTTGGAAGCAGTATGTTGATGGAGATCAGTGTGAGTCCAATCCATGTTTAAATGGCGGCAGT
TGCAAGGATGTCATTAATTCCTATGAATGTTGGTGTCCCTTTGGATTTGAAGGAAAGAACTGTGAATT
AGTGCCATTTCCATGTGGAAGAGTTTCTGTTTCACAAACTTCTAAGCTCACCCGTGCTGAGGCTGTTT
TTCCTGATGTGGACTATGTAAATTCTACTGAAGCTGAAACCATTTTGGATAACATCACTCAAAGCACC
CAATCATTTAATGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAAATGGATTGTAA
CTGCTGCCCACTGTGTTGAAACTGGTGTTAAAATTACAGTTGTCGCAGGTGAACATAATATTGAGGAG
ACAGAACATACAGAGCAAAAGCGAAATGTGATTCGAATTATTCCTCACCACAACTACAATGCAGCTAT
TAATAAGTACAACCATGACATTGCCCTTCTGGAACTGGACGAACCCTTAGTGCTAAACAGCTACGTTA
CACCTATTTGCATTGCTGACAAGGAATACACGAACATCTTCCTCAAATTTGGATCTGGCTATGTAAGT
GGCTGGGGAAGAGTCTTCCACAAAGGGAGATCAGCTTTAGTTCTTCAGTACCTTAGAGTTCCACTTGT
TGACCGAGCCACATGTCTTCGATCTACAAAGTTCACCATCTATAACAACATGTTCTGTGCTGGCTTCC
ATGAAGGAGGTAGAGATTCATGTCAAGGAGATAGTGGGGGACCCCATGTTACTGAAGTGGAAGGGACC
AGTTTCTTAACTGGAATTATTAGCTGGGGTGAAGAGTGTGCAATGAAAGGCAAATATGGAATATATAC
CAAGGTATCCCGGTATGTCAACTGGATTAAGGAAAAAACAAAGCTCACTTGACTGCAGCCAAGCTAAT
TCCGGAAG

| | | | |
|---|---|---|---|
| NOV47e, | | | |
| SNP13382503 of | | | |
| CG55806-04, | SEQ ID NO: 756 | | MW at 47001.8kD |
| Protein Sequence | SNP Pos: 151 | 418 aa | SNP Change: Thr to Ala |

MQRVNMIMAESPGLITICLLGYLLSAECTVFLDHENANKILNRAPKRYNSGKLEEFVGNLERECMEEK
CSFEEAREVFENTERTTEFWKQYVDGDCESNPCLNGGSCKDVINSAYECWCPFGFEGKNCELVPFPCG
RVSVSQTSKLTRAEAVFPDVDYVNSTAEETILDNITQSTQSFNDFTRVVGGEDAKPGQFPWQVVLNGK
VDAFCGGSIVNEKWIVTAAHCVETGVKITVVAGEHNIEETEHTEQKRNVIRIIPHHNYNAAINKYNHD
IALLELDEPLVLNSYVTPICIADKEYTNIFLKFGSGYVSGWGRVFHKGRSALVLQYLRVPLVDRATCL
RSTKFTIYNNMFCAGFHEGGRDSCQGDSGGPHVTEVEGTSFLTGIISWGEECAMKGKYGIYTKVSRYV
NWIKEKTKLT

| | | |
|---|---|---|
| NOV47f, | | |
| SNP13382492 of | SEQ ID NO: 757 | 1300 bp |
| CG55806-04, | ORF Start: at 2 | ORF Stop: at 1253 |
| DNA Sequence | SNP Pos: 673 | SNP Change: G to A |

TGACTGTATCGCCGGATTCATGCAGCGCGTGAACATGATCATGGCAGAATCACCAGGCCTCATCACCA
TCTGCCTTTTAGGATATCTACTCAGTGCTGAATGTACAGTTTTTCTTGATCATGAAAACGCCAACAAA
ATTCTGAATCGGCCAAAGAGGTATAATTCAGGTAAATTGGAGGAGTTTGTTCATGGGAACCTTGAGAG
AGAATGTATGGAAGAAAAGTGCAGTTTTGAAGAAGCACGAGAAGTTTTTGAAAACCTGAAAGAAACAA
CTGAATTTTGGAAGCAGTATGTTGATGGAGATCAGTGGAGTCCAATCCATGTTTAAATAGGCGGCAGT
TGCAAGGATGTCATTAATTCCTATGAATGTTGGTGTCCCTTTGGATTTGAAGGAAAGAACTGTGAATT
AGTGCCATTTCCATGTGGAAGAGTTTCTGTTTCACAAACTTCTAAGCTCACCCGTGCTGAGACTGTTT
TTCCTGATGTGGACTATGTAAATTCTACTGAAGCTGAAACCATTTTGGATAACATCACTCAAAGCACC
CAATCATTTAATGACTTCACTCGGGTTGTTGGTGGAGAAGATGCCAAACCAGGTCAATTCCCTTGGCA
GGTTGTTTTGAATGGTAAAGTTGATGCATTCTGTGGAGGCTCTATCGTTAATGAAAAATGAATTGTAA
CTGCTGCCCACTGTGTTGAAACTGGTGTTAAAATTACAGTTGTCGCAGGTGAACATAATATTGAGGAG
ACAGAACATACAGAGCAAAAGCGAAATGTGATTCGAATTATTCCTCACCACAACTACAATGCAGCTAT
TAATAAGTACAACCATGACATTGCCCTTCTGGAACTGGACGAACCCTTAGTGCTAAACAGTAACGTTA
CACCTATTTGCATTGCTGACAAGGAATACACGAACATCTTCCTCAAATTTGGATCTGGCTATGTAAGT
GGCTGGGGAAGAGTCTTCCACAAAGGGAGATCAGCTTTAGTTCTTCAGTACCTTAGAGTTCCACTTGT
TGACCGAGCCACATGTCTTCGATCTACAAAGTTCACCATCTATAACAACATGTTCTGTGCTGGCTTCC
ATGAAGGAGGTAGAGATTCATGTCAAGGAGATAGTGGGGGACCCCATGTTACTGAAGTGGAAGGGACC
AGTTTCTTAACTGGAATTATTAGCTGGGGTGAAGAGTGTGCAATGAAAGGCAAATATGGAATATATAC
CAAGGTATCCCGGTATGTCAACTGGATTAAGGAAAAAACAAAGCTCACTTGACTGCAGCCAAGCTAAT
TCCGGAAG

| | | | |
|---|---|---|---|
| NOV47f, | | | |
| SNP13382492 of | | | |
| CG55806-04, | SEQ ID NO: 758 | | MW at46845.6kD |
| Protein Sequence | SNP Pos: 218 | 1417 aa | SNP Change: Trp to End |

MQRVNMIMAESPGLITICLLGYLLSAECTVFLDHENANKILNRPKRYNSGKLEEFVHGNLERECMEEK
CSFEEAREVFENTERTTEFWKQYVDGDQCESNPCLNGGSCKDVINSYECWCPFGFEGKNCELVPFPCG
RVSVSQTSKLTRAETVFPDVDYVNSTEAETILDNITQSTQSFNDFTRVVGGEDAKPGQFPWQVVLNGK
VDAFCGGSIVNEKIVTAAHCVETGVKITVVAGEHNIEETEHTEQKRNVIRIIPHHNYNAAINKYNHDI
ALLELDEPLVLNSYVTPICIADKEYTNIFLKFGSGYVSGWGRVFHKGRSALVLQYLRVPLVDRATCLR
STKFTIYNNMFCAGFHEGGRDSCQGDSGGPHVTEVEGTSFLTGIISWGEECAMKGKYGIYTKVSRYVN
WIKEKTKLT

A ClustalW comparison of the above protein sequences yields the following sequence alignment shown in Table 47B.

TABLE 47B

Comparison of the NOV47 protein sequences.

| | |
|---|---|
| NOV47a | MQRVNMIMAESPGLITICLLGYLLSAECTVFLDHENANKILNRPKRYNSGKLEEFVHGNL |
| NOV47b | MQRVNNIMAESPGLITICLLGYLLSABCTVFLDHENANKILNRPKRYNSGKLEEFVQGNL |
| NOV47c | MQRVNMIMABSPGLITICLLGYLLSAECTVFLDHENANKILNRPKRYNSGKLEEFVQGNL |
| NOV47d | MQRVNNIMABSPGLITICLLGYLLSAECTVFLDHENANKILNRPKRYNSGKLEEFVQGNL |
| NOV47a | ERECMEEKCSFEEAREVFENTERTTEFWKQYVDGDQCESNPCLNGGSCKDVINSYECWCP |
| NOV47b | ERECLEEKCSFEEAREVFENTERTTEFWKQYVDGDQCESNPCLNGGSCKDDINSYECWCP |
| NOV47c | ERECMEEKCSFEEAREVFENTERTTEFWKOYVDGDQCESNPCLNGGSCKDDINSYECWCP |
| NOV47d | ERECMEEKCSFEEAREVFENTERTTEFWKQYVDGDQCESNPCLNGGSCKDDINSYECWCP |
| NOV47a | FGFEGKNCELVP-----------------------------------------FP--CGR |
| NOV47b | FGFEGKNCELDV-----------------------------------------D------ |
| NOV47c | FGFEGKNCELDVTCNIKNGRCEQFCKNSADNKVVCSCTEGYRLAENQKSCEPAVPFPCGR |
| NOV47d | FGFEGKNCELDVTCNIKNGRCEQFCKNSADNKVVCSCTEGYRLABNQKSCEPAVPFPCGR |
| NOV47a | VSVSQTSKLTRAETVFPDVDYVNSTEAETILDNITQSTQSFNDFTRVVGGEDAKPGQFPW |
| NOV47b | --------------------YVNSTEAETILDNITQSTQSFNDFTRVVGGEDAKPGQFPW |
| NOV47c | VSVSQTSKLTRAETVFPDVDYVNSTEAETILDNITQSTQSFNDFTRVVGGEDAKPGQFPW |
| NOV47d | VSVSQTSKLTRAETVFPDVDYVNSTEAETILDNITQSTQSFNDFTRVVGGEDAKPGQFPW |
| NOV47a | QVVLNGKVDAFCGGSIVNEKWIVTAAHCVETGVKITVVAGEHNIEETEHTEQKRNVIRII |
| NOV47b | QVVLNGKVDAFCGGSIVNEKWIVTAAHCVETGVKITVVAGEHNIEETEHTEQKRNVIRII |
| NOV47c | QVVLNGKVDAFCGGSIVNEKWIVTAAHCVETGVKITVVAGEHNIEETEHTEQKRNVIRII |
| NOV47d | QVVLNGKVDAFCGGSIVNEKWIVTAAHCVETGVKITVVAGEHNIEETEHTEQKRNVIRII |
| NOV47a | PHHNYNAAINKYNHDIALLELDEPLVLNSYVTPICIADKEYTNIFLKFGSGYVSGWGRVF |
| NOV47b | PHHNYNAAINKYNHDIALLELDEPLVLNSYVTPICIADKEYTNIFLKFGSGYVSGWGRVF |
| NOV47c | PHHNYNAAINKYNHDIALLELDEPLVIMSYVTPICIADKEYTNIFLKFGSGYVSGWGRVF |
| NOV47d | PHHNYNAAINKYNHDIALLELDEPLVLNSYVTPICIADKEYTNIFLKFGSGYVSGWGRVF |
| NOV47a | HKGRSALVLQYLRVPLVDRATCLRSTKFTIYNNMFCAGFHEGGRDSCQGDSGGPHVTEVE |
| NOV47b | HKGRSALVLQYLRVPLVDRATCLRSTKFTIYNNMFCAGFHEGGRDSCQGDSGGPHVTEVE |
| NOV47c | HKGRSALVLQYLRVPLVDRATCLRSTKFTIYNNMFCAGFHEGGRDSCQGDSGGPHVTEVE |
| NOV47d | HKGRSALVLQYLRVPLVDRATCLRSTKFTIYNNMFCAGFHEGGRDSCQGDSGGPHVTEVE |
| NOV47a | GTSFLTGIISWGEECAMKGKYGIYTKVSRYVNWIKEKTKLT |
| NOV47b | GTSFLTGIISWGEECAMKGKYGIYTKVSRYVNWIKEKTKLT |
| NOV47c | GTSFLTGIISWGEECAMKGKYGIYTKVSRYVNWIKEKTKLT |
| NOV47d | GTSFLTGIISWGEECAMKGKYGIYTKVSRYVNWIKEKTKLT |
| NOV47a | (SEQ ID NO: 748) |
| NOV47b | (SEQ ID NO: 750) |
| NOV47c | (SEQ ID NO: 752) |
| NOV47d | (SEQ ID NO: 754) |

Further analysis of the NOV47a protein yielded the following properties shown in Table 47C.

TABLE 47C
Protein Sequence Properties NOV47a

| | |
|---|---|
| SignalP analysis: | Cleavage site between residues 26 and 27 |
| PSORT II analysis: | |

| | |
|---|---|
| PSG: | a new signal peptide prediction method |
| | N-region: length 10; pos.chg 1; neg.chg 1 |
| | H-region: length 16; peak value 9.84 |
| | PSG score: 5.44 |
| GvH: | von Heijne's method for signal seq. recognition |
| | GvH score (threshold: −2.1) : −2.46 |
| | possible cleavage site: between 25 and 26 |

>>> Seems to have no N-terminal signal peptide

| ALOM: | Klein et al's method for TM region allocation |
|---|---|
| | Init position for calculation: 1 |
| | Tentative number of TMS(s) for the threshold 0.5: 1 |
| | Number of TMS(s) for threshold 0.5: 1 |
| | INTEGRAL Likelihood = −2.50 Transmembrane 14-30 |
| | PERIPHERAL Likelihood = 2.07 (at 219) |
| | ALOM score: −2.50 (number of TMSs: 1) |
| MTOP: | Prediction of membrane topology (Hartmann et al.) |
| | Center position for calculation: 21 |
| | Charge difference: −2.5 C(−1.5) - N( 1.0) |
| | N >= C: N-terminal side will be inside |

>>> membrane topology: type 2 (cytoplasmic tail 1 to 14)

| MITDISC: | discrimination of mitochondrial targeting seq | | | |
|---|---|---|---|---|
| | R content: | 1 | Hyd Moment(75): | 4.83 |
| | Hyd Moment(95): | 6.44 | G content: | 2 |
| | D/E content: | 2 | S/T content: | 3 |
| | Score: −6.32 | | | |

Gavel: prediction of cleavage sites for mitochondrial preseq

R-2 motif at 13 QRV|NM

NUCDISC: discrimination of nuclear localization signals

| | pat4: RPKR (4) at 43 |
|---|---|
| | pat7: none |
| | bipartite: none |
| | content of basic residues: 10.0% |
| | NLS Score: −0.22 |
| KDEL: | ER retention motif in the C-terminus: none |

ER Membrane Retention Signals:

XXRR-like motif in the N-terminus: QRVN
KKXX-like motif in the C-terminus: KTKL

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | Prediction: cytoplasmic |
| | Reliability: 94.1 |

```
COIL: Lupas's algorithm to detect coiled-coil regions
  58 N 0.51
  59 L 0.51
  60 E 0.51
  61 R 0.51
  62 E 0.51
  63 C 0.51
  64 M 0.51
  65 E 0.51
  66 E 0.51
  67 K 0.51
  68 C 0.51
  69 S 0.51
  70 F 0.51
  71 E 0.51
  72 E 0.51
  73 A 0.51
  74 R 0.51
  75 E 0.51
  76 V 0.51
  77 F 0.51
  78 E 0.51
  79 N 0.51
  80 T 0.51
  81 E 0.51
  82 R 0.51
  83 T 0.51
  84 T 0.51
  85 E 0.51
```

total: 28 residues

-------------------------
Final Results (k = 9/23):

```
34.8%: mitochondrial
30.4%: cytoplasmic
  8.7%: Golgi
  8.7%: nuclear
  4.3%: vacuolar
  4.3%: extracellular, including cell wall
  4.3%: vesicles of secretory system
  4.3%: endoplasmic reticulum
```

>> prediction for CG55806-04 is mit (k = 23)

A search of the NOV47a protein against the Geneseq database, a proprietary database that contains sequences published in patents and patent publication, yielded several homologous proteins shown in Table 47D.

TABLE 47D
Geneseq Results for NOV47a

| | | NOV47a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB99529 | Amino acid sequence of human | 1..418 | 416/461 (90%) | 0.0 |
| | Factor IX - Homo sapiens, 461 | 1..461 | 416/461 (90%) | |
| | aa. [W020028609]-A2, 31-Oct.- | | | |
| | 2002] | | | |
| ABB81908 | Protein relating to the invention | 1..418 | 416/461 (90%) | 0.0 |
| | #1 - Unidentified, 461 aa. | 1..461 | 416/461 (90%) | |
| | [RU2181147-C2, 10-Apr.-2002] | | | |
| AAY97295 | Human clotting factor IX - Homo | 1..418 | 416/461 (90%) | 0.0 |
| | sapiens, 461 aa. [W0200049147- | 1..461 | 416/461 (90%) | |
| | A1, 24-Aug.-2000] | | | |
| AAO21524 | Protein of human factor IX - | 1..418 | 408/462 (88%) | 0.0 |
| | Homo sapiens, 461 aa. | 1..461 | 409/462 (88%) | |
| | [WO200240544-A2, 23-May- | | | |
| | 2002] | | | |
| ABP53571 | Human NOV3 protein SEQ ID | 1..418 | 391/418 (93%) | 0.0 |
| | NO:6 - Homo sapiens, 394 aa. | 1..394 | 392/418 (93%) | |
| | [WO200262999-A2, 15-Aug.- | | | |
| | 2002] | | | |

In a BLAST search of public sequence databases, the NOV47a protein was found to have homology to the proteins shown in the BLASTP data in Table 47E.

TABLE 47E
Public BLASTP Results for NOV47a

| | | NOV47a | Identities/ | |
|---|---|---|---|---|
| Protein | | Residues/ | Similarities for | |
| Accession | Protein/ | Match | the Matched | Expect |
| Number | /Organism/Length | Residues | Portion | Value |
| CAA00205 | FACTOR IX - | 1..418 | 416/461 (90%) | 0.0 |
| | Homo sapiens | 1..461 | 416/461 (90%) | |
| | (Human), 461 aa. | | | |
| Q95ND7 | Coagulation factor | 1..418 | 415/461 (90%) | 0.0 |
| | XI - Pan | | | |
| | troglodytes | 1..461 | 416/461 (90%) | |
| | (Chimpanzee), | | | |
| | 461 aa. | | | |
| P00740 | Coagulation factor | 1..418 | 415/461 (90%) | 0.0 |
| | IX precursor | 1..461 | 415/461 (90%) | |
| | (EC 3.4.21.22) | | | |
| | (Christmas factor) - | | | |
| | Homo sapiens | | | |
| | (Human), 461 aa. | | | |
| Q14316 | F9 (Coagulation | 6..418 | 411/456 (90%) | 0.0 |
| | factor IX (Plasma | 1..456 | 411/456 (90%) | |
| | THROMBO- | | | |
| | PLASTIC | | | |
| | component, | | | |
| | christmas | | | |
| | disease, | | | |
| | HAEMOPHILIA | | | |
| | B)) | | | |
| | (Factor IX) - | | | |
| | Homo sapiens | | | |
| | (Human), 456 aa. | | | |
| CAA01607 | FACTOR IX | 6..418 | 408/456 (89%) | 0.0 |
| | PROTEIN - Homo | 1..456 | 408/456 (89%) | |
| | sapiens (Human), | | | |
| | 456 aa. | | | |

PFam analysis indicates that the NOV47a protein contains the domains shown in the Table 47F.

TABLE 47F
Domain Analysis of NOV47a

| | | Identities/ | |
|---|---|---|---|
| | NOV47a | Similarities for | |
| Pfam Domain | Match Region | the Matched Region | Expect Value |
| gla | 52..93 | 26/42 (62%) | 1.1e-18 |
| | | 40/42 (95%) | |
| EGF | 97..128 | 16/47 (34%) | 1.9e-06 |
| | | 25/47 (53%) | |
| trypsin | 184..411 | 109/263 (41%) | 4.4e-92 |
| | | 197/263 (75%) | |

### Example 48

The NOV48 clone was analyzed, and the nucleotide and encoded polypeptide sequences are shown in Table 48A.

TABLE 48A

NOV48 Sequence Analysis

| | | |
|---|---|---|
| NOV48a, CG55828-02 | SEQ ID NO: 759 | 1439 bp |
| DNA Sequence | ORF Start: ATG at 35 | ORF Stop: TGA at 1406 |

TCATCCACAGCCATCATATAAAGGTTTTGGCATCATGTTTGGGAAGAAAAAGAAAAAGATTGAAATAT
CTGGCCCGTCCAACTTTGAACACAGGGTTCATACTGGGTTTGATCCACAAGAGCAGAAGTTTACCGGC
CTTCCCCAGCAGTGGCACAGCCTGTTAGCAGATACGGCCAACAGGCCAAAGCCTATGGTGGACCCTTC
ATGCATCACACCCATCCAGCTGGCTCCTATGAAGGTGGATTACGATCGAGCACAGATGGTCCTCAGCC
CTCCACTGTCAGGGTCTGACACCTACCCCAGGGGCCCTGCCAAACTACCTCAAAGTCAAAGCAAATCG
GGCTATTCCTCAAGCAGTCACCAGTACCCGTCTGGGTACCACAAAGCCACCTTGTACCATCACCCCTC
CCTGCAGAGCAGTTCGCAGTACATCTCCACGGCTTCCTACCTGAGCTCCCTCAGCCTCTCATCCAGCA
CCTACCCGCCGCCCAGCTGGGGCTCCTCCTCCGACCAGCAGCCCTCCAGGGTGTCCCATGAACAGTTT
CGGGCGGCCCTGCAGCTGGTGGTCAGCCCAGGAGACCCCAGGGAATACTTGGCCAACTTTATCAAAAT
CGGGGAAGGCTCAACCGGCATCGTATGCATCGCCACCGAGAAACACACAGGGAAACAAGTTGCAGTGA
AGAAAATGGACCTCCGGAAGCAACAGAGACGAGAACTGCTTTTCAATGAGGTCGTGATCATGCGGGAT
TACCACCATGACAATGTGGTTGACATGTACAGCAGCTACCTTGTCGGCGATGAGCTCTGGGTGGTCAT
GGAGTTTCTAGAAGGTGGTGCCTTGACAGACATTGTGACTCACACCAGAATGAATGAAGAACAGATAG
CTACTGTCTGCCTGTCAGTTCTGAGAGCTCTCTCCTACCTTCATAACCAAGGAGTGATTCACAGGGAC
ATAAAAAGTGACTCCATTCTCCTGACAAGCGATGGCCGGATAAAGTTGTCTGATTTTGGTTTCTGTGC
TCAAGTTTCCAAAGAGGTGCCGAAGAGGAAATCATTGGTTGGCACTCCCTACTGGATGGCCCCTGAGG
TGATTTCTAGGCTACCTTATGGGACAGAGGTGGACATCTGGTCCCTCGGGATCATGGTGATAGAAATG
ATTGATGGCGAGCCCCCCTACTTCAATGAGCCTCCCCTCCAGGCGATGCGGAGGATCCGGGACAGTTT
ACCTCCAAGAGTGAAGGACCTACACAAGGTTTCTTCAGTGCTCCGGGGATTCCTAGACTTGATGTTGG
TGAGGGAGCCCTCTCAGAGAGCAACAGCCCAGGAACTCCTCGGACATCCATTCTTAAAACTAGCAGGT
CCACCGTCTTGCATCGTCCCCCTCATGAGACAATACAGGCATCACTGAGCAGAGGATTCGTGTAGGTG
GCAAAGCTAGA

| | | | |
|---|---|---|---|
| NOV48a, | | | |
| CG55828-02 | | | |
| Protein Sequence | SEQ ID NO: 760 | 457 aa | MW at 51352.3kD |

MFGKKKKKIEISGPSNFEHRVHTGFDPQEQKFTGLPQOWHSLLADTANRPKPMVDPSCITPIQLAPMK
VDYDRAQMVLSPPLSGSDTYPRGPAKLPQSQSKSGYSSSSHQYPSGYHKATLYHHPSLQSSSQYISTA
SYLSSLSLSSSTYPPPSWGSSSDQQPSRVSHEQFRAALQLVVSPGDPREYLANFIKIGEGSTGIVCIA
TEKHTGKQVAVKKMDLRKQQRRELLFNEVVIMRDYHHDNVVDMYSSYLVGDELWVVMEFLEGGALTDI
VTHTRNNEEQIATVCLSVLRALSYLHNQGVIHRDIKSDSILLTSDGRIKLSDFGFCAQVSKEVPKRKS
LVGTPYWMAPEVISRLPYGTEVDIWSLGIMVIEMIDGEPPYFNEPPLQAMRRIRDSLPPRVKDLHKVS
SVLRGFLDLMLVREPSQRATAQELLGHPFLKLAGPPSCIVPLMRQYRHH

| | | |
|---|---|---|
| NOV48b, CG55828-01 | SEQ ID NO: 761 | 4796 bp |
| DNA Sequence | ORF Start: ATG at 549 | ORF Stop: TGA at 1833 |

NCGAAAACCCGGAGCAGCTGCGTACGCTCATGGACAGTCCTCCGAGGGGCGAAGCCGGGCAGCTGGGC
ATGCTCAGTAGCTGGGGGAGGTTTGGGTGGAGAGTAGAAAGCTGTGGCTCTGCCTCTCATCCCCTCCC
GCTGGCCCCCGCCCCCCTTGCCCCTACCCAGCCAGTAGTAGTTCCCCAGCGTGCGCCCGGGGAGACCG
GGAACATGGCGCTGGGAGCGCTGTAGCAGCTGAGAAGGGGCTGAGGCACCGCCGCTTCGCTGACAGCC
GGCCACCAGATGTTCATGCATTCTAGAGAAAGTGGAAAACTTAGAAGCCTAATTAATGACTGTCTTCT
GGACCTCTGAGACCATGTTTCTAGTGTTTTCCGTGGAATATTATCAGAAATACACTGTGGTGAAATGC
TTCCACCTCTTGCTAAAATGAACACTGAGGAAAAATGAAGAAGACTGACAAGCACCAGCGAAAAGTTG
CAGAATAGAAATAGCCACACTCCTCTGGAGTCTTTAATTCATCCACAGCCATCATATAAAGGTTTTGG
CATCATGTTTGGGAAGAAAAAGAAAAAGATTGAAATATCTGGCCCGTCCAACTTTGAACACAGGGTTC
ATACTGGGTTTGATCCACAAGAGCAGAAGTTTACCGGCCTTCCCCAGCAGTGGCACAGCCTGTTAGCA
GATACGGCCAACAGGCCAAAGCCTATGGTGGACCCTTCATGCATCACACCCATCCAGCTGGCTCCTAT
GAAGACAATCGTTAGAGGAAACAAACCCTGCAAGGAAACCTCCATCAACGGCCTGCTAGAGGATTTTG
ACAACATCTCGGTGACTCGCTCCAACTCCCTAAGGAAAGAAAGCCCACCCACCCCAGATCAGGGAGCC
TCCAGCCACGGTCCAGGCCACGCGGAAGAAAATGGCTTCATCACCTTCTCCCAGTATTCCAGCGAATC
CGATACTACTGCTGACTACACGACCGAAAAGTACAGGGAGAAGAGTCTCTATGGAGATGATCTGGATC
CGTATTATAGAGGCAGCCACGCAGCCAAGCAAAATGGGCACGTAATGAAAATGAAGCACGGGGAGGCC
TACTATTCTGAGGTGAAGCCTTTGAAATCCGATTTTGCCAGATTTTCTGCCGATTATCACTCACATTT
GGACTCACTGAGCAAACCAAGTGAATACAGTGACCTCAAGTGGGAGTATCAGAGAGCCTCGAGTAGCT
CCCCTCTGGATTATTCATTCCAATTCACACCTTCTAGAACTGCAGGGCCAGCGGGTGCTCCAAAGGAG
AGCCTGGCGTACAGTGAAAGTGAATGGGGACCCAGCCTGGATGACTATGACAGGAGGCCAAAGTCTTC
GTACCTGAATCAGACAAGCCCTCAGCCCACCATGCGGCAGAGGTCCAGGTCAGGCTCGGGACTCCAGG
AACCGATGATGCCATTTGGAGCAAGTGCATTTAAAACCCATCCCCAAGGACACTCCTACAACTCCTAC
ACCTACCCTCGCTTGTCCGAGCCCACAATGTGCATTCCAAAGGTGGATTACGATCCAGAACAGATGGT
CCTCAGCCCTCCACTGTCAGGGTCTGACACCTACCCCAGGGGCCCTGCCAAACTACCTCAAAGTCAAA
GCAAATCGGGCTATTCCTCAAGCAGTCACCAGTACCCGTCTGGGTACCACAAAGCCACCTTGTACCAT
CACCCCTCCCTGCAGAGCAGTTCGCAGTACATCTCCACGGCTTCCTACCTGAGCTCCCTAAGCCTCTC
ATCCAGCATACCCGCCGCCCAGCTGGGGCTCCTCCTCCGACCAGCAGCCCTCCAGGGTGTCCCATGAA
CAGTTTCGGGCGGCCCTGCAGCTGGTGGTCAGCCCAGGAGACCCCAGGGAATACTTGGCCAACTTTAT
CAAAATCGGGGAAGGCTCAACCGGCATCGTATGCATCGCCACCGAGAAACACACAGGGAAACAAGTTG
CAGTGAAGAAAATGGACCTCCGGAAGCAACAGAGACGAGAACTGCTTTTCAATGAGGTCGTGATCATG
CGGGATTACCACCATGACAATGTGGTTGACATGTACAGCAGCTACCTTGTCGGCGATGAGCTCTGGGT
GGTCATGGAGTTTCTAGAAGGTGGTGCCTTGACAGACATTGTGACTCACACCAGAATGAATGAAGAAC
AGATAGCTACTGTCTGCCTGTCAGTTCTGAGAGCTCTGTCCTACCTTCATAACCAAGGAGTGATTCAC
AGGGACATAAAAAGTGACTCCATCCTCCTGACAAGCGATGGCCGGATAAAGTTGTCTGATTTTGGTTT
CTGTGCTCAAGTTTCCAAAGAGGTGCCGAAGAGGAAATCATTGGTTGGCACTCCCTACTGGATGGCCC
CTGAGGTGATTrCTAGGCTACCTTATGGGACAGAGGTGGACATCTGGTCCCTCGGGATCATGGTGATA
GAAATGATTGATGGCGAGCCCCCCTACTTCAATGAGCCTCCCCTCCAGGCGATGCGGAGGATCCGGGA
CAGTTTACCTCCAAGAGTGAAGGACCTACACAAGGTTTCTTCAGTGCTCCGGGGATTCCTAGACTTGA
TGTTGGTGAGGGAGCCCTCTCAGAGAGCAACAGCCCAGGAACTCCTCGGACATCCATTCTTAAAACTA
GCAGGTCCACCGTCTTGCATCGTCCCCCTCATGAGACAATACAGGCATCACTGAGCAGAGGATTCGTG
TAGGTGGCAAAGCTAGATGAGGACATGAGAATAATTCAGGAGAACAAAAGGAAACACAGAACATGCAA
AAGGCCTGTGCATTCTAGACCAGCCAATTGGTGGGACAGCGTGATGACCGGCAGGGTTCAACAGACCA
GGGCATCTTCTTGTGTCTTAAACAGGCATCTCTCCACTGACAGCCGGTGTGGTCACTTGGAGAACGGC
TTTAATAAGTCATTATTATATTTTTCAGCCCTTCATCCAGCAAATCAGAAGGACTCAGTACAAACTCC
GTTATGATATATCCTAGCCACATGCAGGGTAACATGTAGGATTTTCTATATTGAAAGAATACTTTTCT
GGCAAAAAAAAAAAAAAAAAGAAAGAAAGGAAAACAAAAAGCACTTTTTTCTTAATGGTAGCAGTATA
ATGTATTTTGCAACGAATTTGTAATTTTTCTGTACGATAGTTTTGATAATTTATAGTACTTTGATGTC
ATGTAGCCATTGTATCAGTTGAAGTAATACTTGTTTACTAGAGGAGTTTGAACAAAGCCTTTCCTACT
TTTTTATCCCTTTAAGAGAACCAATGATTCTTTAGGAACTTTGAATACTGAATGACTCTCAATCACCG
TCAGCTTTAGTAAAATCTCTTTCTTATCCTAACAAGTGTCTTATTTGGTGGAAGAAGAATTAAGAGTG
ATGGTGATGGTGTGCACGTTTCATTAATCCAACCAAAAATAATGAAATAAAATTTGAGCCACAGTATA
CCACTCCTTGGGATAAAGTTAAATATTTTTAAAGATCACATTTTCCATGAACGCCTCTAGTAGCAAAC
CATTCTTTTGCACACCACAATGTTTCCCTCAGTGCCCTTTCTCAAATGGGTACAATGTTCCCTTGTGG
CACAGTATTAATCGCTATTGTTCATGTATCGTGCTGGAAGTCTGAACTGACTCTAGAGGATGAATTAG
CAAGAGGGTATTTTACCAGGTATGATCTGACTTCAGTTGTGCCCATGTTATAATGTGTTTCCGACATA
CATTTGTCACCATTTTTAAACTCTGTATGCTAGCACACCAAACTCTTGTCTATAATTTACCTTTGTAC
CACAGTATTAATCGCTATTGTTCATGTATCGTGCTGGAAGTCTGAACTGACTCTAGAGGATGAATTAG
CAAGAGGGTATTTTACCAGGTATGATCTGACTTCAGTTGTGCCCATGTTATAATGTGTTTCCGACATA
GGAGAGTCGTGCTGCTGTCTAGATCTTCTTGAATGTTGATAAAAATGAATGACTACTACAATACATTT
TGTGTTGCTTGTTGGATGAATTTGCATGTTAACTGTAGGCCAATATAGATTTGCCTTTAAAACTCTGG
AAGAGCTACATAGTCATCATTAGTTTCTATTAATTATGCATCAGACAAAAGCCATTTGTTACCAAACT
GGGAAAACAGAGGCTTTTCTTAACTATTTCACATACTGTAACAAATATGAATTTAAATTTGTGATAGC
GCTCTGGTTGCTCTAAGCATAATTAAGAATTTTTGTAATTAATAGGTTGCTAATTATTTATCACTGCT
AAAAAGGAAAAAAGGCATAAAATGACCTTCTACTGATTAGATTTTCAGTTTTCTTTCAAACTGGAAAT
GCCTCCATAAATATGATCTATGATTTTGCTTCATAAAACAGCAAATCAATGTTTTATGTAAAATATTA
AAGCATTAATATAAATATGTGAGAATAAAAACAATCTAAATCCAGAAAATGGCAGTCCTAAATGTTCA
TGAGACAGATTGTATTAATTTAACCAGGACTATGTAGAAGTAGAAAGAAAAGAAAAAGAAAATCTTTT
TTAAACCAGAATAAACATTAAAAACTATTGCAGAAAATAGTGGATTTTGGATTCCAAACATTTTCGAC
AGTGTAATGGAAATTTTTCTGTAATTTTCTTACCATCGGGTATTTTTTAAAGTATTCATTGAGTTTAC
CAAAAGTTACTGTAGCTTAAAAGGTTTTGTGAGCACTAACTATTGGCAGAAACTGCATTTGCAAATAA
AAATAAATGTTTGCCTTTTAAAAAAAAAAAAAAAAA

| | | | |
|---|---|---|---|
| NOV48b, CG55828-01 | | | |
| Protein Sequence | SEQ ID NO: 762 | 428 aa | MW at 47439.0kD |

MFGKKKKKIEISGPSNFEHRVHTGFDPQEQKFTGLPQQWHSLLADTANRPKPMVDPSCITPIQLAPMK
TIVRGNKPCKETSINGLLEDFDNISVTRSNSLRKESPPTPDQGASSHGPGHAEENGFITFSQYSSESD
TTADYTTEKYREKSLYGDDLDPYYRGSHAAKQNGHVMKMKHGEAYYSEVKPLKSDFARFSADYHSHLD
SLSKPSEYSDLKWEYQRASSSSPLDYSFQFTPSRTAGTSGCSKESLAYSESEWGPSLDDYDRRPKSSY
LNQTSPQPTMRQRSRSGSGLQEPMMPFGASAFICIHPQGHSYNSYTYPRLSEPLCIPKVDYDPAQHVL
SPPLSGSDTYPRGPAKLPQSOSKSGYSSSSHQYPSGYHKATLYHHPSLQSSSQYISTASYLSSLSLSS
SIPAAQLGLLLRPAALQGVP

| | | |
|---|---|---|
| NOV48c, | | |
| SNP13379517 of | SEQ ID NO: 763 | 1439 bp |
| CG55828-02, | ORF Start: ATG at 35 | ORF Stop: TGA at 1406 |
| DNA Sequence | SNP Pos: 149 | SNP Change: T to C |

TCATCCACAGCCATCATATAAAGGTTTTGGCATCATGTTTGGGAAGAAAAAGAAAAAGATTGAAATAT
CTGGCCCGTCCAACTTTGAACACAGGGTTCATACTGGGTTTGATCCACAAGAGCAGAAGTTTACCGGC
CTTCCCCAGCAGCGGCACAGCCTGTTAGCAGATACGGCCAACAGGCCAAAGCCTATGGTGGACCCTTC
ATGCATCACACCCATCCAGCTGGCTCCTATGAAGGTGGATTACGATCGAGCACAGATGGTCCTCAGCC
CTCCACTGTCAGGGTCTGACACCTACCCCAGGGGCCCTGCCAAACTACCTCAAAGTCAAAGCAAATCG
GGCTATTCCTCAAGCAGTCACCAGTACCCGTCTGGGTACCACAAAGCCACCTTGTACCATCACCCCTC
CCTGCAGAGCAGTTCGCAGTACATCTCCACGGCTTCCTACCTGAGCTCCCTCAGCCTCTCATCCAGCA
CCTACCCGCCGCCCAGCTGGGGCTCCTCCTCCGACCAGCAGCCCTCCAGGGTGTCCCATGAACAGTTT
CGGGCGGCCCTGCAGCTGGTGGTCAGCCCAGGAGACCCCAGGGAATACTTGGCCAACTTTATCAAAAT
CGGGGAAGGCTCAACCGGCATCGTATGCATCGCCACCGAGAAACACACAGGGAAACAAGTTGCAGTGA
AGAAAATGGACCTCCGGAAGCAACAGAGACGAGAACTGCTTTTCAATGAGGTCGTGATCATGCGGGAT
TACCACCATGACAATGTGGTTGACATGTACAGCAGCTACCTTGTCGGCGATGAGCTCTGGGTGGTCAT
GGAGTTTCTAGAAGGTGGTGCCTTGACAGACATTGTGACTCACACCAGAATGAATGAAGAACAGATAG
CTACTGTCTGCCTGTCAGTTCTGAGAGCTCTCTCCTACCTTCATAACCAAGGAGTGATTCACAGGGAC
ATAAAAAGTGACTCCATTCTCCTGACAAGCGATGGCCGGATAAAGTTGTCTGATTTTGGTTTCTGTGC
TCAAGTTTCCAAAGAGGTGCCGAAGAGGAAATCATTGGTTGGCACTCCCTACTGGATGGCCCCTGAGG
TGATTTCTAGGCTACCTTATGGGACAGAGGTGGACATCTGGTCCCTCGGGATCATGGTGATAGAAATG
ATTGATGGCGAGCCCCCCTACTTCAATGAGCCTCCCCTCCAGGCGATGCGGAGGATCCGGGACAGTTT
ACCTCCAAGAGTGAAGGACCTACACAAGGTTTCTTCAGTGCTCCGGGGATTCCTAGACTTGATGTTGG
TGAGGGAGCCCTCTCAGAGAGCAACAGCCCAGGAACTCCTCGGACATCCATTCTTAAAACTAGCAGGT
CCACCGTCTTGCATCGTCCCCCTCATGAGACAATACAGGCATCACTGAGCAGAGGATTCGTGTAGGTG
GCAAAGCTAGA

| | | | |
|---|---|---|---|
| NOV48c, | | | |
| SNP13379517 of | | | |
| CG55828-02, | SEQ ID NO: 764 | | MW at 51322.3kD |
| Protein Sequence | SNP Pos: 39 | 457 aa | SNP Change: Trp to Arg |

MFGKKKKKIEISGPSNFEHRVHTGFDPQEQKFTGLPQQRHSLLADTANRPKPMVDPSCITPIQLAPMK
VDYDRAQMVLSPPLSGSDTYPRGPAKLPQSQSKSGYSSSSHQYPSGYHKATLYHHPSLQSSSQYISTA
SYLSSLSLSSSTYPPPSWGSSSDQQPSRVSHEQFRAALQLVVSPGDPREYLANFIKIGEGSTGIVCIA
TEKHTGKQVAVKKMDLRKQQRRELLFNEVVIMRDYHHDNVVDMYSSYLVGDELWVVMEFLEGGALTDI
VTHTRMNEEQIATVCLSVLRALSYLHNQGVUIRDIKSDSILLTSDGRIKLSDFGFCAQVSKEVPKRKS
LVGTPYWMAPEVISRLPYGTEVDIWSLGIMVIENIDGEPPYFNEPPLQAMRRIRDSLPPRVKDLHKVS
SVLRGFLDLMLVREPSQRATAQELLGHPFLKLAGPPSCIVPLMRQYRHH

| | | |
|---|---|---|
| NOV48d, | | |
| SNP13376535 of | SEQ ID NO: 765 | 1439 bp |
| CG55828-02, | ORF Start: ATG at 35 | ORF Stop: TGA at 1406 |
| DNA Sequence | SNP Pos: 222 | SNP Change: A to G |

TCATCCACAGCCATCATATAAAGGTTTTGGCATCATGTTTGGGAAGAAAAAGAAAAAGATTGAAATAT
CTGGCCCGTCCAACTTTGAACACAGGGTTCATACTGGGTTTGATCCACAAGAGCAGAAGTTTACCGGC
CTTCCCCAGCAGTGGCACAGCCTGTTAGCAGATACGGCCAACAGGCCAAAGCCTATGGTGGACCCTTC
ATGCATCACACCCATCCGGCTGGCTCCTATGAAGGTGGATTACGATCGAGCACAGATGGTCCTCAGCC
CTCCACTGTCAGGGTCTGACACCTACCCCAGGGGCCCTGCCAAACTACCTCAAAGTCAAAGCAAATCG
GGCTATTCCTCAAGCAGTCACCAGTACCCGTCTGGGTACCACAAAGCCACCTTGTACCATCACCCCTC
CCTGCAGAGCAGTTCGCAGTACATCTCCACGGCTTCCTACCTGAGCTCCCTCAGCCTCTCATCCAGCA
CCTACCCGCCGCCCAGCTGGGGCTCCTCCTCCGACCAGCAGCCCTCCAGGGTGTCCCATGAACAGTTT
CGGGCGGCCCTGCAGCTGGTGGTCAGCCCAGGAGACCCCAGGGAATACTTGGCCAACTTTATCAAAAT
CGGGGAAGGCTCAACCGGCATCGTATGCATCGCCACCGAGAAACACACAGGGAAACAAGTTGCAGTGA
AGAAAATGGACCTCCGGAAGCAACAGAGACGAGAACTGCTTTTCAATGAGGTCGTGATCATGCGGGAT
TACCACCATGACAATGTGGTTGACATGTACAGCAGCTACCTTGTCGGCGATGAGCTCTGGGTGGTCAT
GGAGTTTCTAGAAGGTGGTGCCTTGACAGACATTGTGACTCACACCAGAATGAATGAAGAACAGATAG
CTACTGTCTGCCTGTCAGTTCTGAGAGCTCTCTCCTACCTTCATAACCAAGGAGTGATTCACAGGGAC
ATAAAAAGTGACTCCATTCTCCTGACAAGCGATGGCCGGATAAAGTTGTCTGATTTTGGTTTCTGTGC
TCAAGTTTCCAAAGAGGTGCCGAAGAGGAAATCATTGGTTGGCACTCCCTACTGGATGGCCCCTGAGG
TGATTTCTAGGCTACCTTATGGGACAGAGGTGGACATCTGGTCCCTCGGGATCATGGTGATAGAAATG
ATTGATGGCGAGCCCCCCTACTTCAATGAGCCTCCCCTCCAGGCGATGCGGAGGATCCGGGACAGTTT
ACCTCCAAGAGTGAAGGACCTACACAAGGTTTCTTCAGTGCTCCGGGGATTCCTAGACTTGATGTTGG
TGAGGGAGCCCTCTCAGAGAGCAACAGCCCAGGAACTCCTCGGACATCCATTCTTAAAACTAGCAGGT
CCACCGTCTTGCATCGTCCCCCTCATGAGACAATACAGGCATCACTGAGCAGAGGATTCGTGTAGGTG
GCAAAGCTAGA

| | | | |
|---|---|---|---|
| NOV48d, | | | |
| SNP13376535 of | | | |
| CG55828-02, | SEQ ID NO: 766 | | MW at 51380.3kD |
| Protein Sequence | SNP Pos: 63 | 457 aa | SNP Change: Gln to Arg |

MFGKKKKKIEISGPSNFEHRVHTGFDPQEOKFTGLPQQWHSLLADTANRPKPMVDPSCITPIRLAPMK
VDYDRAQMVLSPPLSGSDTYPRGPAKLPQSQSKSGYSSSSHQYPSGYHKATLYHHPSLQSSSQYISTA
SYLSSLSLSSSTYPPPSWGSSSDQQPSRVSHEQFRAALQLVVSPGDPREYLANFIKIGEGSTGIVCIA
TEKHTGQVAVKKMDLRKQQRRELLFNEVVIMRDYHHDNVVDMYSSYLVGDELWVVMEFLEGGAALTDI
VTHTRNNEEQIATVCLSVLRALSYLHNQGVIHRDIKSDSILLTSDGRIKLSDFGFCAQVSKEVPKRKS
LVGTPYWMAPEVISRLPYGTEVDIWSLGIMVIEMIDGEPPYFNEPPLQANRRIRDSLPPRVKDLHKVS
SVLRGFLDLMLVREPSQRATAQELLGHPFLKLAGPPSCIVPLMRQYRHH

| | | |
|---|---|---|
| NOV48e, | | |
| SNP13382500 of | SEQ ID NO: 767 | 1439 bp |
| CG55828-02, | ORF Start: ATG at 35 | ORF Stop: TGA at 1406 |
| DNA Sequence | SNP Pos: 970 | SNP Change: T to C |

TCATCCACAGCCATCATATAAAGGTTTTGGCATCATGTTTGGGAAGAAAAAGAAAAAGATTGAAATAT
CTGGCCCGTCCAACTTTGAACACAGGGTTCATACTGGGTTTGATCCACAAGAGCAGAAGTTTACCGGC
CTTCCCCAGCAGTGGCACAGCCTGTTAGCAGATACGGCCAACAGGCCAAAGCCTATGGTGGACCCTTC
ATGCATCACACCCATCCAGCTGGCTCCTATGAAGGTGGATTACGATCGAGCACAGATGGTCCTCAGCC
CTCCACTGTCAGGGTCTGACACCTACCCCAGGGGCCCTGCCAAACTACCTCAAAGTCAAAGCAAATCG
GGCTATTCCTCAAGCAGTCACCAGTACCCGTCTGGGTACCACAAAGCCACCTTGTACCATCACCCCTC
CCTGCAGAGCAGTTCGCAGTACATCTCCACGGCTTCCTACCTGAGCTCCCTCAGCCTCTCATCCAGCA
CCTACCCGCCGCCCAGCTGGGGCTCCTCCTCCGACCAGCAGCCCTCCAGGGTGTCCCATGAACAGTTT
CGGGCGGCCCTGCAGCTGGTGGTCAGCCCAGGAGACCCCAGGGAATACTTGGCCAACTTTATCAAAAT
CGGGGAAGGCTCAACCGGCATCGTATGCATCGCCACCGAGAAACACACACCCAACAAGTTGCAAGTGA
AGAAAATGGACCTCCGGAAGCAACAGAGACGAGAACTGCTTTTCAATGAGGTCGTGATCATGCGGGAT
TACCACCATGACAATGTGGTTGACATGTACAGCAGCTACCTTGTCGGCGATGAGCTCTGGGTGGTCAT
GGAGTTTCTAGAAGGTGGTGCCTTGACAGACATTGTGACTCACACCAGAATGAATGAAGAACAGATAG
CTACTGTCTGCCTGTCAGTTCTGAGAGCTCTCTCCTACCTTCATAACCAAGGAGTGATTCACAGGGAC
ATAAAAAGTGACTCCATCCTCCTGACAAGCGATGGCCGGATAAAGTTGTCTGATTTTGGTTTCTGTGC
TCAAGTTTCCAAAGAGGTGCCGAAGAGGAAATCATTGGTTGGCACTCCCTACTGGATGGCCCCTGAGG
TGATTTCTAGGCTACCTTATGGGACAGAGGTGGACATCTGGTCCCTCGGGATCATGGTGATAGAAATG
ATTGATGGCGAGCCCCCCTACTTCAATGAGCCTCCCCTCCAGGCGATGCGGAGGATCCGGGACAGTTT
ACCTCCAAGAGTGAAGGACCTACACAAGGTTTCTTCAGTGCTCCGGGGATTCCTAGACTTGATGTTGG
TGAGGGAGCCCTCTCAGAGAGCAACAGCCCAGGAACTCCTCGGACATCCATTCTTAAAACTAGCAGGT
CCACCGTCTTGCATCGTCCCCCTCATGAGACAATACAGGCATCACTGAGCAGAGGATTCGTGTAGGTG
GCAAAGCTAGA

| | | | |
|---|---|---|---|
| NOV48e, | | | |
| SNP13382500 of | | | |
| CG55828-02, | SEQ ID NO: 768 | | MW at 51352.3kD |
| Protein Sequence | SNP Pos: 312 | 457 aa | SNP Change: Ile to Ile |

MFGKKKKKIEISGPSNFEHRVHTGFDPQEQKFTGLPQQWHSLLADTANRPKPMVDPSCITPIQLAPMK
VDYDRAQMVLSPPLSGSDTYPRGPAKLPQSQSKSGYSSSSHQYPSGYHKATLYHHPSLQSSSQYISTA
SYLSSLSLSSSTYPPPSWGSSSDQQPSRVSHEQFRAALQLVVSPGDPREYLANFIKIGEGSTGIVCIA
TEKHTGKQVAVKKMDLRKQQRRELLFNEVVIMRDYHHDNVVDMYSSYLVGDELWVVMEFLEGGALTDI
VTHTRNNEEQIATVCLSVLRALSYLHNQGVIHRDIKSDSILLTSDGRIKLSDFGFCAQVSKEVPKRKS
LVGTPYWMAPEVISRLPYGTEVDIWSLGIMVIEMIDGEPPYFNEPPLQANRRIRDSLPPRVKDLHKVS
SVLRGFLDLMLVREPSQRATAQELLGHPFLKLAGPPSCIVPLMRQYRHH

| | | |
|---|---|---|
| NOV48f, | | |
| SN1P13375705 of | SEQ ID NO: 769 | 1439 bp |
| CG55828-02, | ORF Start: ATG at 35 | ORF Stop: TGA at 1406 |
| DNA Sequence | SNP Pos: 1375 | SNP Change: C to T |

TCATCCACAGCCATCATATAAAGGTTTTGGCATCATGTTTGGGAAGAAAAAGAAAAAGATTGAAATAT
CTGGCCCGTCCAACTTTGAACACAGGGTTCATACTGGGTTTGATCCACAAGAGCAGAAGTTTACCGGC
CTTCCCCAGCAGTGGCACAGCCTGTTAGCAGATACGGCCAACAGGCCAAAGCCTATGGTGGACCCTTC
ATGCATCACACCCATCCAGCTGGCTCCTATGAAGGTGGATTACGATCGAGCACAGATGGTCCTCAGCC
CTCCACTGTCAGGGTCTGACACCTACCCCAGGGGCCCTGCCAAACTACCTCAAAGTCAAAGCAAATCG
GGCTATTCCTCAAGCAGTCACCAGTACCCGTCTGGGTACCACAAAGCCACCTTGTACCATCACCCCTC
CCTGCAGAGCAGTTCGCAGTACATCTCCACGGCTTCCTACCTGAGCTCCCTCAGCCTCTCATCCAGCA
CCTACCCGCCGCCCAGCTGGGGCTCCTCCTCCGACCAGCAGCCCTCCAGGGTGTCCCATGAACAGTTT
CGGGCGGCCCTGCAGCTGGTGGTCAGCCCAGGAGACCCCAGGGAATACTTGGCCAACTTTATCAAAAT
CGGGGAAGGCTCAACCGGCATCGTATGCATCGCCACCGAGAAACACACAGGGAAACAAGTTGCAGTGA
AGAAAATGGACCTCCGGAAGCAACAGAGACGAGAACTGCTTTTCAATGAGGTCGTGATCATGCGGGAT
TACCACCATGACAATGTGGTTGACATGTACAGCAGCTACCTTGTCGGCGATGAGCTCTGGGTGGTCAT
GGAGTTTCTAGAAGGTGGTGCCTTGACAGACATTGTGACTCACACCAGAATGAATGAAGAACAGATAG
CTACTGTCTGCCTGTCAGTTCTGAGAGCTCTCTCCTACCTTCATAACCAAGGAGTGATTCACAGGGAC
ATAAAAAGTGACTCCATTCTCCTGACAAGCGATGGCCGGATAAAGTTGTCTGATTTTGGTTTCTGTGC
TCAAGTTTCCAAAGAGGTGCCGAAGAGGAAATCATTGGTTGGCACTCCCTACTGGATGGCCCCTGAGG
TGATTTCTAGGCTACCTTATGGGACAGAGGTGGACATCTGGTCCCTCGGGATCATGGTGATAGAAATG
ATTGATGGCGAGCCCCCCTACTTCAATGAGCCTCCCCTCCAGGCGATGCGGAGGATCCGGGACAGTTT
CCTCCAAAGAGTGAAGGACCTACACAAGGTTTCTTCAGTGCTCCGGGGATTCCTAGACTTGATGTTGG
TGAGGGAGCCCTCTCAGAGAGCAACAGCCCAGGAACTCCTCGGACATCCATTCTTAAAACTAGCAGGT
CCACCGTCTTGCATTGTCCCCCTCATGAGACAATACAGGCATCACTGAGCAGAGGATTCGTGTAGGTG
GCAAAGCTAGA

| | | | |
|---|---|---|---|
| NOV48f, | | | |
| SNP13375705 of | | | |
| CG55828-02, | SEQ ID NO: 770 | | MW at 51352.3kD |
| Protein Sequence | SNP Pos: 447 | 457 aa | SNP Change: Ile to Ile |

MFGKKKKKIEISGPSNFEHRVHTGFDPQEQKFTGLPQQWHSLLADTANRPKPMVDPSCITPIQLAPMK
VDYDRAQMVLSPPLSGSDTYPRGPAKLPQSQSKSGYSSSSHQYPSGYHKATLYHHPSLQSSSQYISTA
SYLSSLSLSSSTYPPPSWGSSSDQQPSRVSHEQFRAALQLVVSPGDPREYLANFIKIGEGSTGIVCIA
TEKHTGKQVAVKKMDLRKQQRRELLFNEVVIMRDYHHDNVVDMYSSYLVGDELWVVMEFLEGGALTDI
VTHTRMNEEQIATVCLSVLRALSYLHNQGVIHRDIKSDSILLTSDGRIKLSDFGFCAQVSKEVPKRKS
LVGTPYWMAPEVISRLPYGTEVDIWSLGIMVIEMIDGEPPYFNEPPLQANRRIRDSLPPRVKDLHKVS
SVLRGFLDLMILVREPSQRATAQELLGHPFLKLAGPPSCIVPLMRQYRHH

A ClustalW comparison of the above protein sequences yields the following sequence alignment shown in Table 48B.

TABLE 48B

Comparison of the NOV48 protein sequences.

| | |
|---|---|
| NOV48a | MFGKKKKKIEISGPSNFEHRVHTGFDPQEQKFTGLPQQWHSLLADTANRPKPMVDPSCIT |
| NOV48b | MFGKKKKKIEISGPSNFEHRVHTGFDPQEQKFTGLPQQWHSLLADTANRPKPMVDPSCIT |
| NOV48a | PIQLAPMKVDYDRAQMVLSPPLSGSDTYPRGPAKLPQSQSKSGYSSSSHQYPSGYHKATL |
| NOV48b | PIQLAPMKTIVRGNKPCKETSING-LLEDFDNISVTRSNSLRKESPPTPDQGASSHGPGH |
| NOV48a | YHHPSLQSSSQYISTASYLSSLSLSSSTYPPPSWGSSSDQQPSRVSHEQFRAALQLVVSP |
| NOV48b | AEENGFITFSQYSSESD--TTADYTTEKYREKSLYGDDLDPYYRGSHAAKQNGHVMKMKH |
| NOV48a | GDPREYLANFIKIGEGSTGIVCIATEKHTGKQVAVKKMDLRKQQRRELLFNEVVIMRDYH |
| NOV48b | G-----EAYYSEVKPLKSDFARFSADYHSHLDSLSKPSEYS-DLKWEYQRASSSSPLDYS |
| NOV48a | HDNVVDMYS-SYLVGDELWVVMEFLEGGALTDIVTHTRMNEEQIATVCLSVLRALSYLHN |
| NOV48b | FQFTPSRTAGTSGCSKESLAYSESEWGPSLDDYDRRPKSS-------------YLNQTSP |
| NOV48a | QGVIHRDIKSDSILLTSDGRIKLSDFGFCAQVSKEVPKRKSLVGTPYWMAPEVISRLPYG |
| NOV48b | QPTMRQRSRS------GSG-LQEPMMPFGASAFKTHPQGHSYNSYTYPRLSEPTMCIP-- |
| NOV48a | TEVDIWSLGIMVIEMIDGEPPYFNEPPLQAMRRIRDSLPPRVKDLHKVSSVLRGFLDLML |
| NOV48b | -KVDYDPAQMVLSPPLSGSDTYPRGP--AKLPQSQSKSGYSSSSHQYPSGYHKATLYHHP |
| NOV48a | VREPS-QRATAQELLGHPFLKLAGPPSCIVPLMRQYRHH--- |
| NOV48b | SLQSSSQYISTASYLSSLSLSSSIPAAQLGLLLRPAALQGVP |
| NOV48a | (SEQ ID NO: 760) |
| NOV48b | (SEQ ID NO: 762) |

Further analysis of the NOV48a protein yielded the following properties shown in Table 48C.

TABLE 48C
Protein Sequence Properties NOV48a

| | |
|---|---|
| SignalP analysis: | No Known Signal Sequence Indicated |
| PSORT II analysis: | |

| PSG: | a new signal peptide prediction method |
|---|---|
| | N-region: length 10; pos.chg 5; neg.chg 1 |
| | H-region: length 7; peak value −2.19 |
| | PSG score: −6.59 |
| GvH: | von Heijne's method for signal seq. recognition |
| | GvH score (threshold: −2.1): −7.22 |
| | possible cleavage site: between 44 and 45 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) .. fixed |
| | PERIPHERAL Likelihood = 2.54 (at 257) |
| | ALOM score: 2.54 (number of TMSs: 0) |

MITDISC: discrimination of mitochondrial targeting seq

| | | | |
|---|---|---|---|
| R content: | 0 | Hyd Moment (75): | 3.66 |
| Hyd Moment(95): | 6.70 | G content: | 2 |
| D/E content: | 2 | S/T content: | 2 |
| Score: −7.66 | | | |

Gavel: prediction of cleavage sites for mitochondrial preseq

cleavage site motif not found

NUCDISC: discrimination of nuclear localization signals

pat4: KKKK (5) at 4
pat4: KKKK (5) at 5
pat4: PKRK (4) at 336
pat7: PKRKSLV (5) at 336
bipartite: none
content of basic residues: 11.2%
NLS Score: 0.84

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 76.7

COIL: Lupas's algorithm to detect coiled-coil regions

total: 0 residues

--------------------------
Final Results (k = 9/23):

```
78.3%: nuclear
13.0%: cytoplasmic
  4.3%: mitochondrial
  4.3%: peroxisomal
```

>> prediction for CG55828-02 is nuc (k = 23)

A search of the NOV48a protein against the Geneseq database, a proprietary database that contains sequences published in patents and patent publication, yielded several homologous proteins shown in Table 48D.

TABLE 48D
Geneseq Results for NOV48a

| | | NOV48a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB65705 | Novel protein kinase, SEQ ID | 56..457 | 393/402 (97%) | 0.0 |
| | NO: 234 - Homo sapiens, 719 aa. | 319..719 | 393/402 (97%) | |
| | [WO200073469-A2, 07-Dec.- | | | |
| | 2000] | | | |
| AAM38963 | Human polypeptide SEQ ID NO | 56..457 | 392/402 (97%) | 0.0 |
| | 2108 - Homo sapiens, 719 aa. | 319..719 | 393/402 (97%) | |
| | [WO200153312-A1, 26-Jul.- | | | |
| | 2001] | | | |
| AAE02187 | Human p2l-activatedkinase 5 | 56..457 | 391/402 (97%) | 0.0 |
| | (PAK5) protein - Homo sapiens, | 319..719 | 392/402 (97%) | |
| | 719 aa. [WO200136602-A2, 25- | | | |
| | May-2001] | | | |
| AAG67825 | Human P21-active kinase 60 | 56..457 | 355/402 (88%) | 0.0 |
| | protein - Homo sapiens, 547 aa. | 147..547 | 358/402 (88%) | |
| | [CN1298009-A, 06-Jun.-2001] | | | |
| ABG19308 | Novel human diagnostic protein | 150..455 | 253/306 (82%) | e-146 |
| | #19299 -Homo sapiens, 620 aa. | 313..618 | 276/306(89%) | |
| | [WO200175067-A2, 11-Oct.- | | | |
| | 2001] | | | |

In a BLAST search of public sequence databases, the NOV48a protein was found to have homology to the proteins shown in the BLASTP data in Table 48E.

TABLE 48E
Public BLASTP Results for NOV48a

| | | NOV48a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9P286 | Serine/threonine-protein kinase | 56..457 | 393/402 (97%) | 0.0 |
| | PAK 7 (EC 2.7.1.-) (p21-activated | 319..719 | 393/402 (97%) | |
| | kinase 7) (PAK-7) (PAK-5) - | | | |
| | Homo sapiens (Human), 719 aa. | | | |
| Q8TB93 | P21(CDKN1A)-activated kinase | 56..457 | 392/402 (97%) | 0.0 |
| | 7 - Homo sapiens (Human), | 319..719 | 392/402 (97%) | |
| | 719 aa. | | | |
| Q8C015 | Serine/threonine-protein kinase | 56..457 | 382/402 (95%) | 0.0 |
| | PAK 5 - Mus musculus (Mouse), | 319..719 | 389/402 (96%) | |
| | 719 aa. | | | |
| Q8BVB0 | Serine/threonine-protein kinase | 56..457 | 381/402 (94%) | 0.0 |
| | PAK 5 - Mus musculus (Mouse), | 319..719 | 388/402 (95%) | |
| | 719 aa. | | | |
| Q9ULS8 | Hypothetical protein KIAA1142 - | 1..455 | 317/460 (68%) | e-174 |
| | Homo sapiens (Human), 467 aa | 30..465 | 359/460 (77%) | |
| | (fragment). | | | |

PFam analysis indicates that the NOV48a protein contains the domains shown in the Table 48F.

TABLE 48F
Domain Analysis of NOV48a

| | | Identities/ | |
|---|---|---|---|
| Pfam | | Similarities | Expect |
| Domain | NOV48a Match Region | for the Matched Region | Value |
| PBD | 11..69 | 22/64(34%) | 1.3e-10 |
| | | 44/64 (69%) | |
| pkinase | 187..438 | 91/297 (31%) | 7.8e-83 |
| | | 200/297 (67%) | |

### Example 49

The NOV49 clone was analyzed, and the nucleotide and encoded polypeptide sequences are shown in Table 49A.

TABLE 49A

NOV49 Sequence Analysis

| | | |
|---|---|---|
| NOV49a, CG55980-02 | SEQ ID NO: 771 | 972 bp |
| DNA Sequence | ORF Start: ATG at 18 | ORF Stop: TAA at 954 |

CTCATATCTCCCTCATTATGTCTGTTCTCAATAACTCCGAAGTCAAGCTTTTCCTTCTGATTGGGATC
CCAGGACTGGAACATGCCCACATTTGGTTCTCCATCCCCATTTGCCTCATGTACCTGCTTGCCATCAT
GGGCAACTGCACCATTCTCTTTATTATAAAGACAGAGCCCTCGCTTCATGAGCCCATGTATTATTTCC
TTGCCATGTTGGCTGTCTCTGACATGGGCCTGTCCCTCTCCTCCCTTCCTACCATGTTGAGGGTCTTC
TTGTTCAATGCCATGGGAATTTCACCTAATGCCTGCTTTGCTCAAGAATTCTTCATTCATGGATTCAC
TGTCATGGAATCCTCAGTACTTCTAATTATGTCTTTGGACCGCTTTCTTGCCATTCACAATCCCTTAA
GATACAGTTCTATCCTCACTAGCAACAGGGTTGCTAAAATGGGACTTATTTTAGCCATTAGGAGCATT
CTCTTAGTGATTCCATTTCCCTTCACCTTAAGGAGATTAAAATATTGTCAAAAGAATCTTCTTTCTCA
CTCATACTGTCTTCATCAGGATACCATGAAGCTGGCCTGCTCTGACAACAAGACCAATGTCATCTATG
GCTTCTTCATTGCTCTCTGTACTATGCTGGACTTGGCACTGATTGTTTTGTCTTATGTGCTGATCTTG
AAGACTATACTCAGCATTGCATCTTTGGCAGAGAGGCTTAAGGCCCTAAATACCTGTGTCTCCCACAT
CTGTGCTGTGCTCACCTTCTATGTGCCCATCATCACCCTGGCTGCCATGCATCACTTTGCCAAGCACA
AAAGCCCTCTTGTTGTGATCCTTATTGCAGATATGTTCTTGTTGGTGCCGCCCCTTATGAACCCCATT
GTGTACTGTGTAAAGACTCGACAAATCTGGGAGAAGATCTTGGGGAAGTTGCTTAATGTATGTGGGAG
ATAAGAACTTGAACAATTAG

| | | | |
|---|---|---|---|
| NOV49a, CG55980-02 | | | |
| Protein Sequence | SEQ ID NO: 772 | 312 aa | MW at 35131.1kD |

MSVLNNSEVKLFLLIGIPGLEHAHIWFSIPICLMYLLAIMGNCTILFIIKTEPSLHEPMYYFLAMLAV
SDMGLSLSSLPTMLRVFLFNAMGISPNACFAQEFFIHGFTVMESSVLLIMSLDRFLAIHNPLRYSSIL
TSNRVAXMGLILAIRSILLVIPFPFTLRRLKYCQKNLLSHSYCLHQDTMKLACSDNKTNVIYGFFIAL
CTMLDLALIVLSYVLILKTILSIASLAERLKALNTCVSHICAVITFYVPIITLAAMHHFAKHKSPLVV
ILIADMFLLVPPLNNPIVYCVKTRQIWEKILGKLLNVCGR

| | | |
|---|---|---|
| NOV49b, CG55980-01 | SEQ ID NO: 773 | 972 bp |
| DNA Sequence | ORF Start: ATG at 18 | ORF Stop: TAA at 954 |

CTCATATCTCCCTCATTATGTCTGTTCTCAATAACTCCGAAGTCAAGCTTTTCCTTCTGATTGGGATC
CCAGGACTGGAACATGCCCACATTTGGTTCTCCATCCCCATTTGCCTCATGTACCTGCTTGCCATCAT
GGGCAACTGCACCATTCTCTTTATTATAAAGACAGAGCCCTCGCTTCATGAGCCCATGTATTATTTCC
TTGCCATGTTGGCTGTCTCTGACATGGGCCTGTCCCTCTCCTCCCTTCCTACCATGTTGAGGGTCTTC
TGTCATGGAATCCTCAGTACTTCTAATTATGTCTTTGGACCGCTTTCTTGCCATTCACAATCCCTTAA
TGTCATGGAATCCTCAGTACTTCTAATTATGTCTTTGGACCGCTTTCTTGCCATTCACAATCCCTTAA
GATACAGTTCTATCCTCACTAGCAACAGGGTTGCTAAAATGGGACTTATTTTAGCCATTAGGAGCATT
CTCTTAGTGATTCCATTTCCCTTCACCTTAAGGAGATTAAAATATTGTCAAAAGAATCTTCTTTCTCA
CTCATACTGTCTTCATCAGGATACCATGAAGCTGGCCTGCTCTGACAACAAGACCAATGTAATCTATG
GCTTcTTCATTGCTCTCTGTACTATGCTGGACTTGGCACTGATTGTTTTGTCTTATGTGCTGATCTTG
AAGACTATACTCAGCATTGCATCTTTGGCAGAGAGGCTTAAGGCCCTAAATACCTGTGTCTCCCACAT
CTGTGCTGTGCTCACCTTCTATGTGCCCATCATCACCCTGGCTGCCATGCATCACTTTGCCAAGCACA
AAAGCCCTCTTGTTGTGATCCTTATTGCAGATATGTTCTTGTTGGTGCCGCCCCTTATGAACCCCATT
GTGTACTGTGTAAAGACTCGACAAATCTGGGAGAAGATCTTGGGGAAGTTGCTTAATGTATGTGGGAG
ATAAGAACTTGAACAATTAG

| | | | |
|---|---|---|---|
| NOV49b, GG55980-01 | | | |
| Protein Sequence | SEQ ID NO: 774 | 312 aa | MW at 35131.1kD |

MSVLNNSEVKLFLLIGIPGLEHAHIWFSIPICLMYLLAIMGNCTILFIIKTEPSLHEPMYYFLAMLAV
SDMGLSLSSLPTMLRVFLFNANGISPNACFAQEFFIHGFTVMESSVLLIMSLDRFLAIHNPLRYSSIL
TSNRVAXMGLILAIRSILLVIPFPFTLRRLKYCQKNLLSHSYCLHQDTMKLACSDNKTNVIYGFFIAI
CTMLDLALIVLSYVLILKTILSIASLAERLKALNTCVSHICAVLTFYVPIITLAAMHHFAKHKSPLVV
ILIADMFLLVPPLMNPIVYCVKTRQIWEKILGKLLNVCGR

A ClustalW comparison of the above protein sequences yields the following sequence alignment shown in Table 49B.

TABLE 49B

Comparison of the NOV49 protein sequences.

| | |
|---|---|
| NOV49a | MSVLNNSEVKLFLLIGIPGLEHAHIWFSIPICLMYLLAIMGNCTILFIIKTEPSLHEPMY |
| NOV49b | MSVLNNSEVKLFLLIGIPGLEHAHIWFSIPICLMYLLAIMGNCTILFIIKTEPSLHEPMY |
| NOV49a | YFLAMLAVSDMGLSLSSLPTMLRVFLFNAMGISPNACFAQEFFIHGFTVMESSVLLIMSL |
| NOV49b | YFLAMLAVSDMGLSLSSLPTMLRVFLFNAMGISPNACFAQEFFIHGFTVMESSVLLIMSL |
| NOV49a | DRFLAIHNPLRYSSILTSNRVAKMGLILAIRSILLVIPFPFTLRRLKYCQKNLLSHSYCL |
| NOV49b | DRFLAIHNPLRYSSILTSNRVAKMGLILAIRSILLVIPFPFTLRRLKYCQKNLLSHSYCL |
| NOV49a | HQDTMKLACSDNKTNVIYGFFIALCTMLDLALIVLSYVLILKTILSIASLAERLKALNTC |
| NOV49b | HQDTMKLACSDNKTNVIYGFFIALCTMLDLALIVLSYVLILKTILSIASLAERLKALNTC |
| NOV49a | VSHICAVLTFYVPIITLAAMHHFAKHKSPLVVILIADMFLLVPPLMNPIVYCVKTRQIWE |
| NOV49b | VSHICAVLTFYVPIITLAAMHHFAKHKSPLVVILIADMFLLVPPLMNPIVYCVKTRQIWE |
| NOV49a | KILGKLLNVCGR |
| NOV49b | KILGKLLNVCGR |
| NOV49a | (SEQ ID NO: 772) |
| NOV49b | (SEQ ID NO: 774) |

Further analysis of the NOV49a protein yielded the following properties shown in Table 49C.

TABLE 49C
Protein Sequence Properties NOV49a

| | |
|---|---|
| SignalP analysis: | Cleavage site between residues 42 and 43 |

PSORT II analysis:

| PSG: | a new signal peptide prediction method |
|---|---|
| | N-region: length 10; pos.chg 1; neg.chg 1 |
| | H-region: length 10; peak value 10.00 |
| | PSG score: 5.60 |
| GvH: | von Heijne's method for signal seq. recognition |
| | GvH score (threshold: −2.1): −3.28 |
| | possible cleavage site: between 23 and 24 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 8 | |
| | INTEGRAL Likelihood = −6.00 | Transmembrane 33-49 |
| | INTEGRAL Likelihood = 0.32 | Transmembrane 62-78 |
| | INTEGRAL Likelihood = −0.48 | Transmembrane 102-118 |
| | INTEGRAL Likelihood = −5.63 | Transmembrane 141-157 |
| | INTEGRAL Likelihood = −7.59 | Transmembrane 199-215 |
| | INTEGRAL Likelihood = 0.42 | Transmembrane 218-234 |
| | INTEGRAL Likelihood = −5.79 | Transmembrane 244-260 |
| | INTEGRAL Likelihood = −8.17 | Transmembrane 270-286 |

| | PERIPHERAL Likelihood = 2.12 (at 11) |
|---|---|
| | ALOM score: −8.17 (number of TMSs: 8) |
| MTOP: | Prediction of membrane topology (Hartmann et al.) |
| | Center position for calculation: 40 |
| | Charge difference: −1.5 C(−0.5) - N( 1.0) |
| | N >= C: N-terminal side will be inside |
| | >>> membrane topology: type 3a |

MITDISC: discrimination of mitochondrial targeting seq

| | | | |
|---|---|---|---|
| R content: | 0 | Hyd Moment (75): | 0.68 |
| Hyd Moment (95): | 3.43 | G content: | 2 |
| D/E content: | 2 | S/T content: | 2 |

Score: −8.46

Gavel: prediction of cleavage sites for mitochondrial preseq

cleavage site motif not found

NUCDISC: discrimination of nuclear localization signals

pat4: none
pat7: none
bipartite: none
content of basic residues: 7.7%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LKYCQKNLLSHSYCLHQDTMKL at 166

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

--------------------------
Final Results (k = 9/23):

66.7%: endoplasmic reticulum
22.2%: mitochondrial
11.1%: vesicles of secretory system

>> prediction for CG55980-02 is end (k = 9)

A search of the NOV49a protein against the Geneseq database, a proprietary database that contains sequences published in patents and patent publication, yielded several homologous proteins shown in Table 49D.

TABLE 49D
Geneseq Results for NOV49a

| | | NOV49a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU85302 | G-coupled olfactory | 1..312 | 312/312 (100%) | e-178 |
| | receptor #163 - Homo | 1..312 | 312/312 (100%) | |
| | sapiens, 312 aa. | | | |
| | [WO200198526-A2, 27- | | | |
| | Dec.-2001] | | | |
| AAU95741 | Human olfactory and | 1..312 | 312/312 (100%) | e-178 |
| | pheromone Gprotein- | 1..312 | 312/312 (100%) | |
| | coupled receptor #228 - | | | |
| | Homo sapiens, 312 aa. | | | |
| | [WO200224726-A2, 28- | | | |
| | Mar.-2002] | | | |
| AAB71167 | Human GPCRX protein | 1..312 | 312/312 (100%) | e-178 |
| | SEQ ID 10 - Homo | 1..312 | 312/312 (100%) | |
| | sapiens, 312 aa. | | | |
| | [WO200250275-A2, 27- | | | |
| | Jun.-2002] | | | |
| AAB71166 | Human GPGRX protein | 1..312 | 312/312 (100%) | e-178 |
| | SEQ ID 8 - Homo sapiens, | 1..312 | 312/312 (100%) | |
| | 312 aa. [WO200250275- | | | |
| | A2, 27-Jun.-2002] | | | |
| AAB71344 | Human GCREC-23 | 1..312 | 312/312 (100%) | e-178 |
| | INCYTE ID 7472087GD1 | 1..312 | 312/312 (100%) | |
| | SEQ ID 23 - Homo | | | |
| | sapiens, 312 aa. | | | |
| | [WO200263004-A2, 15- | | | |
| | Aug.-2002] | | | |

In a BLAST search of public sequence databases, the NOV49a protein was found to have homology to the proteins shown in the BLASTP data in Table 49E.

TABLE 49E
Public BLASTP Results for NOV49a

| | | NOV49a | Identities/ | |
|---|---|---|---|---|
| Protein | | Residues/ | Similarities for | Ex- |
| Accession | Protein/ | Match | the Matched | pect |
| Number | /Organism/Length | Residues | Portion | Value |
| Q8NH64 | Seven trans- | 1..312 | 312/312 (100%) | e-178 |
| | membrane helix | 1..312 | 312/312 (100%) | |
| | receptor - Homo | | | |
| | sapiens (Human), | | | |
| | 312 aa. | | | |
| Q8VH16 | Olfactory receptor | 1..306 | 242/308 (78%) | e-141 |
| | MOR8-1 - | 8..315 | 272/308 (87%) | |
| | Mus musculus | | | |
| | (Mouse), 318 | | | |
| | aa. | | | |
| Q8VH12 | Olfactory receptor | 3..306 | 233/306 (76%) | e-134 |
| | MOR8-3 - | 2..307 | 262/306 (85%) | |
| | Mus musculus | | | |
| | (Mouse), 312 | | | |
| | aa. | | | |
| GAD37707 | Sequence 449 from | 1..306 | 222/308 (72%) | e-130 |
| | Patent | 1..308 | 268/308 (86%) | |
| | WO0224726 - | | | |
| | Homo sapiens | | | |
| | (Human), 313 aa. | | | |
| Q8NGJ6 | Seven trans- | 1..306 | 221/308 (71%) | e-129 |
| | membrane helix | 1..308 | 267/308 (85%) | |
| | receptor - | | | |
| | Homo sapiens | | | |
| | (Human), 313 aa. | | | |

PFam analysis indicates that the NOV49a protein contains the domains shown in the Table 49F.

TABLE 49F
Domain Analysis of NOV49a

| | NOV49a | Identities/ | |
|---|---|---|---|
| | Match | Similarities | |
| Pfam Domain | Region | for the Matched Region | Expect Value |
| 7tm_1 | 41..291 | 57/277 (21%) | 7.4e-13 |
| | | 169/277 (61%) | |

### Example 50

The NOV50 clone was analyzed, and the nucleotide and encoded polypeptide sequences are shown in Table 50A.

TABLE 50A

NOV50 Sequence Analysis

| | | |
|---|---|---|
| NOV50a, CG55988-03 | SEQ ID NO: 775 | 1630 bp |
| DNA Sequence | ORF Start: ATG at 1 | ORF Stop: TGA at 1528 |

ATGGGGTCCCGCCACTTCGAGGGGATTTATGACCACGTGGGGCACTTCGGCAGATTCCAGAGAGTCCT
CTATTTCATATGTGCCTTCCAGAACATCTCTTGTGGTATTCACTACTTGGCTTCTGTGTTCATGGGAG
TCACCCCTCATCATGTCTGCAGGCCCCCAGGCAATGTGAGTCAGGTTGTTTTCCATAATCACTCTAAT
TGGAGTTTGGAGGACACCGGGGCCCTGTTGTCTTCAGGCCAGAAAGATTATGTTACGGTGCAGTTGCA
GAATGGTGAGATCTGGGAGCTCTCAAGGTGTAGCAGGAATAAGAGGGAGAACACATCGAGTTTGGGCT
ATGAATACACTGGCAGTAAGAAAGAGTTTCCTTGTGTGGATGGCTACATATATGACCAGAACACATGG
AAAAGCACTGCGGTGACCCAGTGGAACCTGGTCTGTGACCGAAAATGGCTTGCAATGCTGATCCAGCC
CCTATTTATGTTTGGAGTCCTACTGGGATCGGTGACTTTTGGCTACTTTTCTGAGGCTAAGGAACGCC
GGGTGGTCTTGTGGGCCACAAGCAGTAGCATGTTTTTGTTTGGAATAGCAGCGGCGTTTGCAGTTGAT
TATTACACCTTCATGGCTGCTCGCTTTTTTCTTGCCATGGTTGCAAGTGGCTATCTTGTGGTGGGGTT
TGTCTATGTGATGGAATTCATTGGCATGAAGTCTCGGAAATGGGCGTCTGTCCATTTGCATTCCTTTT
TTGCAGTTGGAACCCTGCTGGTGGCTTTGACAGGATACTTGGTCAGGACCTGGTGGCTTTACCAGATG
ATCCTCTCCACAGTGACTGTCCCCTTTATCCTGTGCTGTTGGGTGCTCCCAGAGACACCTTTTTGGCT
TCTCTCAGAGGGACGATATGAAGAAGCACAAAAAATAGTTGACATCATGGCCAAGTGGAACAGGGCAA
GCTCCTGTAAACTGTCAGAACTTTATCACTGGACCTACAAGGTCCTGTTAGTAATAGCCCCACTGAAT
GTTCAGAAGCACAACCTATCATATCTGTTTTATAACTGGAGCATTACGAAAAGGACACTTACCGTTTG
GCTAATCTGGTTCACTGGAAGTTTGGGATTCTACTCGTTTTCCTTGAATTCTGTTAACTTAGGAGGCA
ATGAATACTTAAACCTCTTCCTCCTGGGTGTAGTGGAAATTCCCGCCTACACCTTCGTGTGCATCGCC
ACGGACAAGGTCGGGAGGAGAACAGTCCTGGCCTACTAACTTTTCTGCAGTGCACTGGCCTGTGGTGT
CGTTATGGTGATCCCCCAGATCGCTGGCTGTGGGAAGCGGCAGCATGGTGTGTCGCCTGGCCAGCATC
CTGGCGCCGTTCTCTGTGGACCTCAGCAGCATTTGGATCTTCATACCACAGTTGTTTGTTGGGACTAT
GGCCCTCCTGAGTGGAGTGTTAACACTAAAGCTTCCAGAAACCCTTGGGAAACGGCTAGCAACTACTT
GGGAGGAGGCTGCAAAACTGGAGTCAGAGAATGAAAGCAAGTCAAGCAAATTACTTCTCACAACTAAT
AATAGTGGGCTGGAAAAAACGGAAGCGATTACCCCCAGGGATTCTGGTCTTGGTGAATAAATGTGC

| | | | |
|---|---|---|---|
| NOV50a, CG55988-03 | | | |
| Protein Sequence | SEQ ID NO: 776 | 509 aa | MW at 57300.6kD |

MGSRHFEGIYDHVGHPGRPQRVLYFICAFQNISCGIHYLASVFMGVTPHHVCRPPGNVSQVVFHNHSN
WSLEDTGALLSSGQKDYVTVQLQNGEIWELSRCSRNKRENTSSLGYEYTGSKKEFPCVDGYIYDQNTW
KSTAVTQWNLVCDRKWLAMLIQPLFMFGVLLGSVTFGYFSDRLGRRVVLWATSSSMFLFGIAAAFAVD
YYTFMAARFFLAMVASGYLVVGFVYVMEFIGMKSRTWASVHLHSFFAVGTLLVALTGYLVRTWWLYQM
ILSTVTVPFILCCWVLPETPFWLLSEGRYEEAQKIVDIMAKWNRASSCKLSELLSLDLQGPVSNSPTE
VQKHNLSYLFYNWSITKRTLTVWLIWFTGSLGFYSFSLNSVNLGGNEYLNLFLLGVVEIPAYTFVCIA
TDKVGRRTVLAYSLFCSALACGVVMVIPQIAGCGKRQHGVSPGQHPGAVLCGPQQHLDLHTTVVCWDY
GPPEWSVNTKASRNPWETASNYLGGGCKTGVRE

| | | |
|---|---|---|
| NOV50b, GG55988-04 | SEQ ID NO: 777 | 1599 bp |
| DNA Sequence | ORF Start: ATG at 1 | ORF Stop: TAA at 1546 |

ATGGGGTCCCGCCACTTCGAGGGGATTTATGACCACGTGGGGCACTTCGGCAGATTCCAGAGAGTCCT
CTATTTCATATGTGCCTTCCAGAACATCTCTTGTGGTATTCACTACTTGGCTTCTGTGTTCATGGGAG
TCACCCCTCATCATGTCTGCAGGCCCCCAGGCAATGTGAGTCAGGTTGTTTTCCATAATCACTCTAAT
TGGAGTTTGGAGGACACCGGGGCCCTGTTGTCTTCAGGCCAGAAAGATTATGTTACGGTGCAGTTGCA
GAATGGTGAGATCTGGGAGCTCTCAAGGTGTAGCAGGAATAAGAGGGAGAACACATCGAGTTTGGGCT
ATGAATACACTGGCAGTAAGAAAGAGTTTCCTTGTGTGGATGGCTACATATATGACCAGAACACATGG
AAAAGCACTGCGGTGACCCAGTGGAACCTGGTCTGTGACCGAAAATGGCTTGCAATGCTGATCCAGCC
CCTATTTATGTTTGGAGTCCTACTGGGATCGGTGACTTTTGGCTACTTTTCTGACAGGCTAGGACGCC
GGGTGGTCTTGTGGGCCACAAGCAGTAGCATGTTTTTGTTTGGAATAGCAGCGGCGTTTGCAGTTGAT
TATTACACCTTCATGGCTGCTCGCTTTTTTCTTGCCATGGTTGCAAGTGGCTATCTTGTGGTGGGGTT
TGTCTATGTGATGGAATTCATTGGCATGAAGTCTCGGACATGGGCGTCTGTCCATTTGCATTCCTTTT
TTGCAGTTGGAACCCTGCTGGTGGCTTTGACAGGATACTTGGTCAGGACCTGGTGGCTTTACCAGATG
ATCCTCTCCACAGTGACTGTCCCCTTTATCCTGTGCTGTTGGGTGCTCCCAGAGACACCTTTTTGGCT
TCTCTCAGAGGGACGATATGAAGAAGCACAAAAAATAGTTGACATCATGGCCAAGTGGAACAGGGCAA
GCTCCTGTAAACTGTCAGAACTTTTATCACTGGACCTACAAGGTCCTGTTAGTAATAGCCCCACTGAA
GTTCAGAACACAACCTATCATATCTGTTTTATAACTGGAGCATTACGAAAAGGACACTTAACCGTTTG
GCTAATCTGGTTCACTGGAAGTTTGGGATTCTACTCGTTTTCCTTGAATTCTGTTAACTTAGGAGGCA
ATGAATACTTAAACCTCTTCCTCCTGGGTGTAGTGGAAATTCCCGCCTACACCTTCGTGTGCATCGCC
ACGGACAAGGTCGGGAGGAGAACAGTCCTGGCCTACTCTCTTTTCTGCAGTGCACTGGCCTGTGGTGT
CGTTATGGTGATCCCCCAGAAACATTATATTTTGGGTGTGGTGACAGCTATGGTTGGAAAATTTGCCA
TCGGGGCAGCATTTGGTCTCATTTATCTTTATACAGCTGAGCTGTATCCAACCATTGTAAGATCGCTG
GCTGTGGGAAGCGGCAGCATGGTGTGTCGCCTGGCCAGCATCCTGGCGCCGTTCTCTGTGGACCTCAG
CAGCATTTGGATCTTCATACCACAGCTCCTAGGACAGCACCTTCAGGAGTAATAATGCAGGCAGTCGC
TGGCAATTAAGGAGGCGCTCCATGGTGGGTCATGA

| | | | |
|---|---|---|---|
| NOV50b, CG55988-04 | | | |
| Protein Sequence | SEQ ID NO: 778 | 515 aa | MW at 58030.0kD |

MGSRHFEGIYDHVGHFGRFQRVLYFICAFQNISCGIHYLASVFMGVTPHHVCRPPGNVSQVVFHNGSN
WSLEDTGALLSSGQKDYVTVQLQNGEIWELSRCSRNKRENTSSLGYEYTGSKKEFPCVDGYIYDQNTW
KSTAVTQWNLVCDRKWLAMLIQPLFMFGVLLGSVTFGYFSDRLGRRVVLWATSSSMFLFGIAAAFAVD
YYTFMAARFFLAMVASGYLVVGFVYVMEFIGMKSRTWASVHLHSFFAVGTLLVALTGYLVRTWWLYQM
ILSTVTVPFILCCWVLPETPFWLLSEGRYEEAQKIVDIMAKWNRASSCKLSELLSLDLQGPVSNSPTE
VQKHNLSYLFYNWSITKRTLTVWLIWFTGSLGFYSFSLNSVNLGGNEYLNLFLLGVVEIPAYTFVCIA
TDKVGRRTVLAYSLFCSALACGVVMVIPQKHYILGVVTAMVGKFAIGAAFGLEYLETAELYTIVARSL
AVGSGSMVCRLASILAPFSVDLSSIWIFIPQLLGQHLQE

| | | |
|---|---|---|
| NOY50c, CG55988-01 | SEQ ID NO: 779 | 2069 bp |
| DNA Sequence | ORF Start: ATG at 279 | ORF Stop: TAA at 1881 |

GCTTCTAGGCCTTCTCAGTAGATGGAGCTAAGTAATATATGTATATATACTAACCCACAGATATAAAT
ATGCTATAATTATTTCTATATTTATCCATTCGTGTATATGTTAAGATAAACATGATGGAGACCCATTC
AAATTTGCTTATGTTCTTTTTCAGCCTATAGACCAGATATAATAATTAGCTTTTCTTCTCTTGCAGAT
TCCAGAGAGTCCTCTATTTCATATGTGCCTTCCAGAACATCTCTTGTGGTATTCACTACTTGGCTTCT
GTGTTCATGGGAGTCACCCCTCATCATGTCTGCAGGCCCCCAGGCAATGTGAGTCAGGTTGTTTTCCA
TAATCACTCTAATTGGAGTTTGGAGGACACCGGGGCCCTGTTGTCTTCAGGCCAGAAAGATTATGTTA
CGGTGCAGTTGCAGAATGGTGAGATCTGGGAGCTCTCAAGGTGTAGCAGGAATAAGAGGGAGAACACA
TCGAGTTTGGGCTATGAATACACTGGCAGTAAGAAAGAGTTTCCTTGTGTGGATGGCTACATATATGA
CCAGAACACATGGAAAAGCACTGCGGTGACCCAGTGGAACCTGGTCTGTGACCGAAATGAGCTTGCAA
TGCTGATCCAGCCCCTATTTATGTTTGGAGTGCCTACTGGGATCGGTGACTTTTGGCTACTTTCTGAC
AGGCTAGGACGCCGGGTGGTCTTGTGGGCCACAAGCAGTAGCATGTTTTTGTTTGGAATAGCAGCGGC
GTTTGCAGTTGATTATTACACCTTCATGGCTGCTCGCTTTTTTCTTGCCATGGTTGCAAGTGGCTATC
TTGTGGTGGGGTTTTGTCTATGTGATGGAATTCATTGGCATGAAGTCTCGGACATGGGCTCTGTCCAT
TTGCATTCCTTTTTTGCAGTTGGAACCCTGCTGGTGGCTTTGACAGGATACTTGGTCAGGACCTGGTG
GCTTTACCAGATGATCCTCTCCACAGTGACTGTCCCCTTTATCCTGTGCTGTTGGGTGCTCCCAGAGA
CACCTTTTTGGCTTCTCTCAGAGGGACGATATGAAGAAGCACAAAAAATAGTTGACATCATGGCCAAG
TGGAACAGGGCAAGCTCCTGTAAACTGTCAGAACTTTTATCACTGGACCTACAAGGTCCTGTTAGTAA
TAGCCCCACTGAAGTTCAGAAGCACAACCTATCATATCTGTTTTATAACTGGAGCATTACGAAAAGGA
CACTTACCGTTTGGCTAATCTGGTTCACTGGAAGTTTGGGATTCTACTCGTTTTCCTTGAATTCTGTT
AACTTAGGAGGCAATGAATACTTAAACCTCTTCCTCCTGGGTGTAGTGGAAATTCCCGCCTACACCTT
CGTGTGCATCGCCATGGACAAGGTCGGGAGGAGAACAGTCCTGGCCTACTCTCTTTTCTGCAGTGCAC
TGGCCTGTGGTGTCGTTATGGTGATCCCCCAGAAACATTATATTTTGGGTGTGGTGACAGCTATGGTT
GGAAAATTTGCCATCGGGGCAGCATTTGGCCTCATTTATCTTTATACAGCTGAGCTGTATCCAACCAT
TGTAAGATCGCTGGCTGTGGGAACGGCAGCATGGTGTGTCGCCTGGCCAGCATCCTGGCAGCCGTTCT
CTGTGGACCTCAGCAGCATTTGGATCTTCATACCACAGTTGTTTGTTGGGACTATGGCCCTCCTGAGT
GGAGTGTTAACACTAAAGCTTCCAGAAACCCTTGGGAAACGGCTAGCAACTACTTGGGAGGAGGCTGC
AAAACTGGAGTCAGAGAATGAAAGCAAGTCAAGCAAATTACTTCTCACAACTAATAATAGTGGGCTGG
AAAAAACGGAAGCGATTACCCCCAGGGATTCTGGTCTTGGTGAATAAATGTGCCATGCCTGGTGTCTA
GCACCTGAAATATTATTTACCCCTAATGCCTTTGTATTAGAGGAATCTTATTCTCATCTCCATATGTT
GTTTGTATGTCTTTTTAATAAATTTTGTAAGAAAATTTTAAAGCAAATATGTTATAAAAGAAATAAAA
ACTAAGATGAAAATTCTCAGTTTTAAAAA

| | | | |
|---|---|---|---|
| NOV50c, GG55988-01 | | | |
| Protein Sequence | SEQ ID NO: 780 | 534 aa | MW at 59606.7kD |

MGVTPHHVCRPPGNVSQVVGHNHSNWSLEDTGALLSSGQKDYVTVQLQNGEIWELSRCSRNKRENTSS
LGYEYTGSKKEFPCVDGYIYDQNTWKSTAVTQWNLVCDRKWLAMLIQPLFMFGVLLGSVTFGYFSDRL
GRRVVLWATSSSMFLFGIAAAFAVDYYTFMAARFFLAMVASGYLVVGFVYVMEFIGMKSRTWASVHLH
RASSCKLSELLSLDLQGPVSNSPTEVQKHNLSYLFYNWSITKRTLTVWLIWFTGSLGFYSFSLNSVML
GGNEYLNLFLLGVVEIPAYTFVCIAMDKVGRRTVLAYSLFCSALACGVVMVIPQKHYILGVVTANVGK
FAIGAAGFLIYLYTAELYPTIVRSLAVGSGSMVCRLASILAPFSVDLSSIWIFIPQLFVGTMALLSGV
LTLKLPETLGKRLATTWEEAAKLESENESKSSKLLLTTNNSGLEKTEAITPRDSGLGE

| | | |
|---|---|---|
| NOV50d, GG55988-02 | SEQ ID NO: 781 | 1666 bp |
| DNA Sequence | ORF Start: ATG at 76 | ORF Stop: TAA at 1654 |

TTCCAGAGAGTCCTCTATTTCATATGTGCCTTCCAGAACATCTCTTGTGGTATTCACTACTTGGCTTC
TGTGTTCATGGGAGTCACCCCTCATCATGTCTGCAGGCCCCCAGGCAATGTGAGTCAGGTTGTTTTCC
ATAATCACTCTAATTGGAGTTTGGAGGACACCGGGGCCCTGTTGTCTTCAGGCCAGAAAGATTATGTT
ACGGTGCAGTTGCAGAATGGTGAGATCTGGGAGCTCTCAAGGTGCAGATGGAATAAGAGGGAGAACAC
ATCGAGTTTGGGCTATGAATACACTGGCAGTAAGAAAGAGTTTCCTTGTGTGGATGGCTACATATATG
ACCAGAACACATGGAAAAGCACTGCGGTGACCCAGTGGAACCTGGTCTGTGACCGAAAATCGGTTGCA
ATGCTGATCCAGCCCCTATTTATGTTTGGAGTCCTACTGGGATGCGGTGACTTTTGGCTACTTTCTGA
CAGGCTTTTTTGCCTATATGTGATTTGCAATGGGTCAGACTCCTCAATAGTTATAAATGTGAACCTTG
AATATAAATCCCTATTATTTGTTTTTCAGGTTGCAAGTGGCTATCTTGTGGTGGGGTTTGTCTATGTG
ATGGAATTCATTGGCATGAAGTCTCGGACATGGGCGTCTGTCCATTTGCATTCCTTTTTGCAAGTTGG
AACCCTGCTGGTGGCTTTGACAGGATACTTGGTCAGGACCTGGTGGCTTTACCAGATGATCCTCTCCA
CAGTGACTGTCCCCTTTATCCTGTGCTGTTGGGTGCTTCCCAGAGACACCTTTTGGCTTCTCTGCAGA
GGACGATATGAAGAAGCACAAAAAATAGTTGACATCATGGCCAAGTGGAACAGGGCAAGCTCCTGTAA
ACTGTCAGAACTTTTATCACTGGACCTACAAGGTCCTGTTAGTAATAGCCCACTGAAGTTCAGAAGCA
ACAACCTATCATATCTGTTTTATAACTGGAGCATTACGAAAAGGACACTTACCGTTTGGCTAATCTGG
TTCACTGGAAGTTTGGGATTCTACTCGTTTTCCTTGAATTCTGTTAACTTAGGAGGCAATGAATACTT
AAACCTCTTCCTCACAGGTGTAGTGGAAATTCCCGCCTACACCTTCGTGTGCATCGCCATGGACAAGG
TCGGGAGGAGAACAGTCCTGGCCTACTCTCTTTTCTGCAGTGCACTGGCCTGTGGTGTCGTTATGGTG
ATCCCCCAGGTGAGTTATCTTCTGGGTGTGGTGACAGCTATGGTTGGAAAATTTGCCATCGGGGCAGC
ATTTGGCCTCATTTATCTTTATACAGCTGAGCTGTATCCAACCATTGTAAGGTCGCTGGCTGTGGGAA
GCGGCAGCATGGTGTGTCGCCTGGCCAGCATCCTGGCGCCGTTCTCTGTGGACCTCAGCAGCATTTGG
ATCTTCATACCACAGTTGTTTGTTGGGACTATGGCCCTCCTGAGTGGAGTGTTAACACTAAAGCTTCC
AGAAACCCTTGGGAAACGGCTAGCAACTACTTGGGACCAGGCTGCAAAACTGGAGTCAGAGAATGAAA
GCAAGTCAAGCAAATTACTTCTCACAACTAATAATAGTGGGCTGGAAAAAACGGAAGCGATTACCCCC
AGGGATTCTGGTCTTGGTGAATAAATGTGCCATG

| | | | |
|---|---|---|---|
| NOV50d, CG55988-02 | | | |
| Protein Sequence | SEQ ID NO: 782 | 526 aa | MW at 58820.8kD |

MGVTPHHVCRPPGNVSQVVFHNHSNWSLEDTGALLSSGQKDYVTVQLQNGEIWELSRCSRNKRENTSS
LGYEYTGSKKEFPCVDGYIYDQNTWSTAVTQWNLVCDRKWLAMLIQPLFMFGVLLGSVTFGYAFSDRL
FCLYVICNGVRLLNSYKCDLEYKSLLFVFQVASGYLVVGFVYVMEFIGMKSRTWASVHLHSFFAVGTL
LVALTGYLVRTWWLYQMILSTVTVPFILCCWVLPETPFWLLSEGRYEEAQKIVDIMAKWNRASSCKLS
ELLSLDLQGPVSNSPTEVQKHNLSYLFYNWSITKRTLTVWLIWFTGSLGYSFSLNSVNLGGNEYALNL
FLTGVVEIPAYTFVCIAMDKVGRRTVLAYSLFCSALACGVVMVIPQVSYLLGVVTAMVGKFAIGAAFG
LIYLYTAELYPTIVRSLAVGSGSMVCRLASILAPFSVDLSSIWIFIPQLFVGTMALLSGVLTLKLPET
LGKRLATTWEEAAKLESENESKSSKLLLTTNNSGLEKTEAITPRDSGLGE

A ClustalW comparison of the above protein sequences yields the following sequence alignment shown in Table 50B.

TABLE 50B

Comparison of the NOV50 protein sequences.

| | |
|---|---|
| NOV50a | MGSRHFEGIYDHVGHFGRFQRVLYFICAFQNISCGIHYLASVFMGVTPHHVCRPPGNVSQ |
| NOV50b | MGSRHFEGIYDHVGHFGRFQRVLYFICAFQNISCGIHYLASVFMGVTPHHVCRPPGNVSQ |
| NOV50c | -------------------------------------------MGVTPHHVCRPPGNVSQ |
| NOV50d | -------------------------------------------MGVTPHHVCRPPGNVSQ |
| NOV50a | VVFHNHSNWSLEDTGALLSSGQKDYVTVQLQNGEIWELSRCSRNKRENTSSLGYEYTGSK |
| NOV50b | VVFHNHSNWSLEDTGALLSSGQKDYVTVQLQNGEIWELSRCSRNKRENTSSLGYEYTGSK |
| NOV50c | VVFHNHSNWSLEDTGALLSSGQKDYVTVQLQNGEIWELSRCSRNKRENTSSLGYEYTGSK |
| NOV50d | VVFHNHSNWSLEDTGALLSSGQKDYVTVQLQNGEIWELSRCSRNKRENTSSLGYEYTGSK |
| NOV50a | KEFPCVDGYIYDQNTWKSTAVTQWNLVCDRKWLAMLIQPLFMFGVLLGSVTFGYFSDRLG |
| NOV50b | KEFPCVDGYIYDQNTWKSTAVTQWNLVCDRKWLAMLIQPLFMFGVLLGSVTFGYFSDRLG |
| NOV50c | KEFPCVDGYIYDQNTWKSTAVTQWNLVCDRKWLAMLIQPLFMFGVLLGSVTFGYFSDRLG |
| NOV50d | KEFPCVDGYIYDQNTWKSTAVTQWNLVCDRKWLANLIQPLFMFGVLLGSVTFGYFSDRLF |
| NOV50a | RRVVLWATSSSMFLFGIAAAFAVDYYTFMAARFFLAMVASGYLVVGFVYVMEFIGMKSRT |
| NOV50b | RRVVLWATSSSMFLFGIAAAFAVDYYTFMAARFFLAMVASGYLVVGFVYVMEFIGMKSRT |
| NOV50c | RRVVLWATSSSMFLFGIAAAFAVDYYTFMAARFFLANVASGYLVVGFVYVMEFIGMKSRT |
| NOV50d | CLYVICN--------GVRLLNSYKCDLEYKSLLFVFQVASGYLVVGFVYVMEFIGMKSRT |
| NOV50a | WASVHLHSFFAVGTLLVALTGYLVRTWWLYQMILSTVTVPFILCCWVLPETPFWLLSEGR |
| NOV50b | WASVHLHSFFAVGTLLVALTGYLVRTWWLYQMILSTVTVPFILCCWVLPETPFWLLSEGR |
| NOV50c | WASVHLHSFFAVGTLLVALTGYLVRTWWLYQMILSTVTVPFILCCWVLPETPFWLLSEGR |
| NOV50d | WASVHLHSFFAVGTLLVALTGYLVRTWWLYQMILSTVTVPFILCCWVLPETPFWLLSEGR |
| NOV50a | YEEAQKIVDIMAKWNRASSCKLSELLSLDLQGPVSNSPTEVQKHNLSYLFYNWSITKRTL |
| NOV50b | YEEAQKIVDIMAKWNRASSCKLSELLSLDLQGPVSNSPTEVQKHNLSYLFYNWSITKRTL |
| NOV50c | YEEAQKIVDIMAKWNRASSCKLSELLSLDLQGPVSNSPTEVQKHNLSYLFYNWSITKRTL |
| NOV50d | YEEAQKIVDIMAKWNRASSCKLSELLSLDLQGPVSNSPTEVQKHNLSYLFYNWSITKRTL |
| NOV50a | TVWLIWFTGSLGFYSFSLNSVNLGGNEYLNLFLLGVVEIPAYTFVCIATDKVGRRTVLAY |
| NOV50b | TVWLIWFTGSLGFYSFSLNSVNLGGNEYLNLFLLGVVEIPAYTFVCIATDKVGRRTVLAY |
| NOV50c | TVWLIWFTGSLGFYSFSLNSVNLGGNEYLNLFLLGVVEIPAYTFVCIAMDKVGRRTVLAY |
| NOV50d | TVWLIWFTGSLGFYSFSLNSVNLGGNEYLNLFLTGVVEIPAYTFVCIAMDKVGRRTVLAY |
| NOV50a | SLFCSALACGVVMVIPQI--------AGCGKRQHGVSPG--------QHP--------G- |
| NOV50b | SLFCSALACGVVMVIPQKHYILGVVTAMVGKFAIGAAFGLIYLYTAELYPTIVRSLAVGS |
| NOV50c | SLFCSALACGVVMVIPQKHYILGVVTAMVGKFAIGAAFGLIYLYTAELYPTIVRSLAVGS |
| NOV50d | SLFCSALACGVVMVIPQVSYLLGVVTAMVGKFAIGAAFGLIYLYTAELYPTIVRSLAVGS |
| NOV50a | -AVLCGPQQHLDLHTTVVC--WDYGP------------------PE---------WSVNT |
| NOV50b | GSMVCRLASILAPFSVDLSSIWIFIPQLLGQHLQE------------------------- |
| NOV50c | GSMVCRLASILAPFSVDLSSIWIFIPQLFVGTMALLSGVLTLKLPETLGKRLATTWEEAA |
| NOV50d | GSMVCRLASILAPFSVDLSSIWIFIPQLFVGTMALLSGVLTLKLPETLGKRLATTWEEAA |
| NOV50a | KASRNPWETASNYLGGGCKTGVRE------------- |
| NOV50b | ------------------------------------- |
| NOV50c | KLESENESKSSKLLLTTNNSGLEKTEAITPRDSGLGE |
| NOV50d | KLESENESKSSKLLLTTNNSGLEKTEAITPRDSGLGE |
| NOV50a | (SEQ ID NO: 776) |
| NOV50b | (SEQ ID NO: 778) |
| NOV50c | (SEQ ID NO: 780) |
| NOV50d | (SEQ ID NO: 782) |

Further analysis of the NOV50a protein yielded the following properties shown in Table 50C.

TABLE 50C
Protein Sequence Properties NOV50a

| | |
|---|---|
| SignalP analysis: | No Known Signal Sequence Indicated |

PSORT II analysis:

| | |
|---|---|
| PSG: | a new signal peptide prediction method |

N-region: length 11; pos.chg 1; neg.chg 2
H-region: length 6; peak value 0.00
PSG score: −4.40

| | |
|---|---|
| GvH: | von Heijne's method for signal seq. recognition |
| | GvH score (threshold: −2.1): −1.51 |
| | possible cleavage site: between 34 and 35 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 7 | |
| | INTEGRAL Likelihood =−3.03 | Transmembrane 154-170 |
| | INTEGRAL Likelihood =−1.12 | Transmembrane 183-199 |
| | INTEGRAL Likelihood =−4.57 | Transmembrane 214-230 |
| | INTEGRAL Likelihood =−3.08 | Transmembrane 248-264 |
| | INTEGRAL Likelihood =−5.89 | Transmembrane 272-288 |
| | INTEGRAL Likelihood =−4.51 | Transmembrane 391-407 |
| | INTEGRAL Likelihood =−5.10 | Transmembrane 419-435 |

| | PERIPHERAL Likelihood = 2.81 (at 360) |
|---|---|
| | ALOM score: −5.89 (number of TMSs: 7) |
| MTOP: | Prediction of membrane topology (Hartmann et al.) |
| | Center position for calculation: 161 |
| | Charge difference: 0.0 C( 2.0) - N( 2.0) |
| | N >= C: N-terminal side will be inside |

>>> membrane topology: type 3a
MITDISC: discrimination of mitochondrial targeting seq

| | | | |
|---|---|---|---|
| R content: | 1 | Hyd Moment (75): | 7.38 |
| Hyd Moment (95): | 9.73 | G content: | 2 |
| D/E content: | 2 | S/T content: | 1 |
| Score: −6.19 | | | |

Gavel: prediction of cleavage sites for mitochondrial preseq

R-2 motif at 14 SRH|FE

NUCDISC: discrimination of nuclear localization signals

pat4: none
pat7: none
bipartite: none
content of basic residues: 7.5%
NLS Score: −0.47

KDEL: ER retention motif in the C-terminus: none
ER Membrane Retention Signals:

XXRR-like motif in the N-terminus: GSRH

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylatiOn pattern : none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LVCDRKWLAMLIQPLFMFGVLL at 146

none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

--------------------------
Final Results (k = 9/23):

77.8%: endoplasmic reticulum
22.2%: mitochondrial

>> prediction for CG55988-03 is end (k = 9)

A search of the NOV50a protein against the Geneseq database, a proprietary database that contains sequences published in patents and patent publication, yielded several homologous proteins shown in Table 50D.

TABLE 50D
Geneseq Results for NOV50a

| | | NOV50a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP74100 | Human TRICH SEQ ID NO 5 - | 1..437 | 436/437 (99%) | 0.0 |
| | Homo sapiens, 577 aa. | 1..437 | 436/437 (99%) | |
| | [WO200246415-A2, 13-Jun.- | | | |
| | 2002] | | | |
| AAM00930 | Human bone marrow protein, | 1..437 | 436/437 (99%) | 0.0 |
| | SEQ ID NO: 406 - Homo sapiens, | 7..443 | 436/437 (99%) | |
| | 584 aa. [WO200153453-A2, 26- | | | |
| | Jul.-2001] | | | |
| AAM78367 | Human protein SEQ ID NO 1029 | 1..437 | 436/437 (99%) | 0.0 |
| | - Homo sapiens, 577 aa. | 1..437 | 43 6/437 (99%) | |
| | [WO200157190-A2, 09-Aug.- | | | |
| | 2001] | | | |
| AAM79351 | Human protein SEQ ID NO 2997 | 1..437 | 432/438 (98%) | 0.0 |
| | - Homo sapiens, 585 aa. | 7..444 | 432/438 (98%) | |
| | [WO200157190-A2, 09-Aug.- | | | |
| | 2001] | | | |
| AAB43038 | Human OREX 0RiF2802 | 18..437 | 419/420 (99%) | 0.0 |
| | polypeptide sequence SEQ ID | 1..420 | 419/420 (99%) | |
| | NO:5604 - Homo sapiens, 560 aa. | | | |
| | [WO200058473-A2, 05-Oct.- | | | |
| | 2000] | | | |

In a BLAST search of public sequence databases, the NOV50a protein was found to have homology to the proteins shown in the BLASTP data in Table 50E.

TABLE 50E
Public BLASTP Results for NOV50a

| | | NOV50a | Identities/ | |
|---|---|---|---|---|
| Protein | | Residues/ | Similarities for | Ex- |
| Accession | Protein/ | Match | the Matched | pect |
| Number | /Organism/Length | Residues | Portion | Value |
| Q96RU0 | Organic cation | 1..437 | 437/437 (100%) | 0.0 |
| | transporter | 1..437 | 437/437 (100%) | |
| | OKB1 - Homo | | | |
| | sapiens (Human), | | | |
| | 577 aa. | | | |
| AAH47565 | Organic cation | 1..437 | 436/437 (99%) | 0.0 |
| | transporter OKB1 - | 1..437 | 436/437 (99%) | |
| | Homo sapiens | | | |
| | (Human), 577 aa. | | | |
| Q8IZD5 | Putative | 1..437 | 436/437 (99%) | 0.0 |
| | transmembrane | 1..437 | 436/437 (99%) | |
| | transporter FLIPT | | | |
| | 2 - Homo | | | |
| | sapiens (Human), | | | |
| | 577 aa. | | | |
| 014567 | WUGSC: | 18..437 | 419/420 (99%) | 0.0 |
| | RG331P03.1 | | | |
| | protein - | | | |
| | Homo sapiens | 1..420 | 419/420 (99%) | |
| | (Human), 456 aa | | | |
| | (fragment). | | | |
| QSIUG8 | Carnitine transporter | 18..437 | 387/420 (92%) | 0.0 |
| | 2 - Homo | 16..403 | 387/420 (92%) | |
| | sapiens (Human), | | | |
| | 543 aa. | | | |

PFam analysis indicates that the NOV50a protein contains the domains shown in the Table 50F.

TABLE 50F
Domain Analysis of NOV50a

| | | Identities/ | |
|---|---|---|---|
| | NOV50a | Similarities for | |
| Pfam Domain | Match Region | the Matched Region | Expect Value |
| sugar_tr | 120..499 | 67/493 (14%) | 7.8e-05 |
| | | 242/493 (49%) | |

### Example 51

The NOV51 clone was analyzed, and the nucleotide and encoded polypeptide sequences are shown in Table 51A.

TABLE 51A
NOV51 Sequence Analysis

| | | |
|---|---|---|
| NOV51a, CG56071-01 | SEQ ID NO: 783 | 3092 bp |

| | | |
|---|---|---|
| DNA Sequence | ORF Start: ATG at 1 | ORF Stop: TAG at 3073 |

ATGGAGCCCTCCAGAGCGCTTCTCGGCTGCCTAGCGAGCGCCGCCGCTGCCGCCCCGCCGGGGGAGGA
TGGAGCAGGGGCCGGGGCCGAGGAGGAGGAGGAGGAGGAGGAGGAGGCGGCGGCGGCGGTGGGCCCCG
GGGAGCTGGGCTGCGACGCGCCGCTGCCCTACTGGACGGCCGTGTTCGAGTACGAGGCGGCGGGCGAG
GACGAGCTGACCCTGCGGCTGGGCGACGTGGTGGAGGTGCTGTCCAAGGACTCGCAGGTGTCCGGCGA
CGAGGGCTGGTGGACCGGGCAGCTGAACCAGCGGGTGGGCATCTTCCCCAGCAACTACGTGACCCCGC
GCAGCGCCTTCTCCAGCCGCTGCCAGCCCGGCGGCGAAATTGATTTTGCGGAGCTCACCTTGGAAGAG
ATTATTGGCATCGGGGGCTTTGGGAAGGTCTATCGTGCTTTCTGGATAGGGGATGAGGTTGCTGTGAA
AGCAGCTCGCCACGACCCTGATGAGGACATCAGCCAGACCATAGAGAATGTTCGCCAAGAGGCCAAGC
TCTTCGCCATGCTGAAGCACCCCAACATCATTGCCCTAAGAGGGGTATGTCTGAAGGAGCCCAACCTC
TGCTTGGTCATGGAGTTTGCTCGTGGAGGACCTTTGAATAGAGTGTTATCTGGGAAAAGGATTCCCCC
AGACATCCTGGTGAATTGGGCTGTGCAGATTGCCAGAGGGATGAACTACTTACATGATGAGGCAATTG
TTCCCATCATCCACCGCGACCTTAAGTCCAGCAACGTATTGATCCTCCAGAAGGTGGAGAATGGAGAC
CTGAGCAACAAGATTCTGAAGATCACTGATTTTGGCCTGGCTCGGGAATGGCACCGAACCACCAAGAT
GAGTGCGGCAGGGACGTATGCTTGGATGGCACCCGAAGTCATCCGGGCCTCCATGTTTTCCAAAGGCA
GTGATGTGTGGAGCTATGGGGTGCTACTTTGGGAGTTGCTGACTGGTGAGGTGCCCTTTCGAGGCATT
GATGGCTTAGCAGTCGCTTATGGAGTGGCCATGAACAAACTCGCCCTTCCTATTCCTTCTACGTGCCC
AGAACCTTTTGCCAAACTCATGGAAGACTGCTGGAATCCTGATCCCCACTCACGACCATCTTTCACGA
ATATCCTGGACCAGCTAACCACCATAGAGGAGTCTGGTTTCTTTGAAATGCCCAAGGACTCCTTCCAC
TGCCTGCAGGACAACTGGAAACACGAGATTCAGGAGATGTTTGACCAACTCAGGGCCAAAGAAAAGGA
ACTTCGCACCTGGGAGGAGGAGCTGACGCGGGCTGCACTGCAGCAGAAGAACCAGGAGGAACTGCTGC
GGCGTCGGGAGCAGGAGCTGGCCGAGCGGGAGATTGACATCCTGGAACGGGAGCTCAACATCATCATC
CACCAGCTGTGCCAGGAGAAGCCCCGGGTGAAGAAACGCAAGGGCAAGTTCAGGAAGAGCCGGCTGAA
GCTCAAGGATGGCAACCGCATCAGCCTCCCTTCTGGTTTCCAGCACAAGTTCACGGTGCAGGCCTCCC
CTACCATGGATAAAAGGAAGAGTCTTATCAACAGCCGCTCCAGTCCTCCTGCAAGCCCCACCATCATT
CCTCGCCTTCGAGCCATCCAGTGTGAGACTGTTTCCCAAATTAGCTGGGGCCAGAACACACAGGGGCA
CCTGTCCGAAAGCAGCAAAACCTGGGGCAGGAGCTCAGTCGTCCCAAAGGAGGAAGGGGAGGAGGAGG
AGAAGAGGGCCCCAAAGAAGAAGGGACGGACGTGGGGGCCAGGGACGCTTGGTCAGAAGGAGCTTGCC
TCGGGAGATGAACTCAAGTCCCTGGTAGATGGATATAAGCAGTGGTCGTCCAGTGCCCCCAACCTGGT
GAAGGGCCCAAGGAGTACCCCGGCCCTGCCAGGGTTCACCAGCCTTATGGAGATGGAGGATGAGGACA
GTGAAGGCCCAGGGAGTGGAGAGAGTCGCCTACAGCATTCACCCAGCCAGTCCTACCTCTGTATCCCA
TTCCCTCGTGGAGAGCCCACCCCAGTCAACTCGGCCACGAGTACCCCTCAGCTGACGCCAACCAACAG
CCTCAAGCGGGGCGGTGCCCACCACCGCCGCTGCGAGGTGGCTCTGCTCGGCTGTGGGGCTGTTCTGG
CAGCCACAGGCCTAGGGTTTGACTTGCTGGAAGCTGGCAAGTGCCAGCTGCTTCCCCTGGAGGAGCCT
GAGCCACCAGCCCGGGAGGAGAAGAAAAGACGGGAGGGTCTTTTTCAGAGGTCCAGCCGTCCTCGTCG
GAGCACCAGCCCCCCATCCCGAAAGCTTTTCAAGAAGGAGGAGCCCATGCTGTTGCTAGGAGACCCCT
CTGCCTCCCTGACGCTGCTCTCCCTCTCCTCCATCTCCGAGTGCAACTCCACACGCTCCCTGCTGCAG
TCCGACAGCGATGAAATTGTCGTGTATGAGATGCCAGTCAGCCCAGTCGAGGCCCCTCCCCTGAGTCC
ATGTACCCACAACCCCCTGGTCAATGTCCGAGTAGAGCGCTTCAAACGAGATCCTAACCAATCTCTGA
CTCCCACCCATGTCACCCTCACCACCCCCTCGCAGCCCAGCAGTCACCGGCGGACTCCTTCTGATGGG
GCCCTTAAGCCAGAGACTCTCCTAGCCAGCAGGAGCCCCAGTCCCAGCCGAGACCCAGGTGAATTCCC
CCGTCTCCCTGACCCCAATGTGGTCTTCCCCCCAACCCCAAGGCGCTGGAACACTCAGCAGGACTCTA
CCTTGGAGAGACCCAAGACTCTGGAGTTTCTGCCTCGGCCGCGTCCTTCTGCCAACCGGCAACGGCTG
GACCCTTGGTGGTTTGTGTCCCCCAGCCATGCCCGCAGCACCTCCCCAGCCAACAGCTCCAGCACAGA
GACGCCCGGGCCGCTGCCCCCGACTGAGCGGACGCTCCTGGACCTGGATGCAGAGGGGCAGAGTCAGG
ACAGCACCGTGCCGCTGTGCAGAGCGGAACTGAACACACACAGGCCTGCCCCTTATGAGATCCAGCAG
GAGTTCTGGTCTTAGCACGAAAAGGATTGGGG

NOV51a, CG56071-01

| | | | |
|---|---|---|---|
| Protein Sequence | SEQ ID NO: 784 | 1024 aa | MW at 113682.1kD |

MEPSRALLGCLASAAAAAPPGEDGAGAGAEEEEEEEEEAAAAVGPGELGCDAPLPYWTAVFEYEAAGE
DELTLRLGDVVEVLSKDSQVSGDEGWWTGQLNQRVGIFPSNYVTPRSAFSSRCQPGGEIDFAELTLEE
IIGIGGFGKVYRAFWIGDEVAVKAARHDPDEDISQTIENVRQEAKLFAMLKHPNIIALRGVCLKEPNL
CLVMEFARGGPLNRVLSGKRIPPDILVNWAVQIARGMNYLHDEAIVPIIHRDLKSSNVLILQKVENGD
LSNKILKITDFGLAREWHRTTKMSAAGTYAWMAPEVIRASMFSKGSDVWSYGVLLWELLTGEVPFRGI
DGLAVAYGVANNKLALPIPSTCPEPFAKLMEDCWNPDPHSRPSFTNILDQLTTIEESGFFEMPKDSFH
CLQDNWKHEIQEMFDQLRAKEKELRTWEEELTRAALQQKNQEELLRRREQELAEREIDILERELNIII
HQLCQEKPRVKKRKGKFRKSRLKLKDGNRISLPSGFQHKFTVQASPTMDKRKSLINSRSSPPASPTII
PRLRAIQCETVSQISWGQNTQGMLSESSKTWGRSSVVPKEEGEEEEKRAPKKKGRTWGPGTLGQKELA
SGDELKSLVDGYKQWSSSAPNLVKGPRSTPALPGFTSLMEMEDEDSEGPGSGESRLQHSPSQSYLCIP
FPRGEPTPVNSATSTPQLTPTNSLKRGGAHHRRCEVALLGCGAVLAATGLGFDLLEAGKCQLLPLEEP
EPPAREEKKRREGLFQRSSRPRRSTSPPSRKLFKKEEPMLLLGDPSASLTLLSLSSISECNSTRSLLQ
SDSDEIVVYEMPVSPVEAPPLSPCTHNPLVNVRVERFKRDPNQSLTPTHVTLTTPSQPSSHRRTPSDG
ALKPETLLASRSPSPSRDPGEFPRLPDPNVVFPPTPRRWNTQQDSTLERPKTLEFLPRPRPSANRQRL
DPWWFVSPSHARSTSPANSSSTETPGPLPPTERTLLDLDAEGOSQDSTVPLCRAELNTHRPAPYEIQQ
EFWS

| | | |
|---|---|---|
| NOV51b, 274082270 | SEQ ID NO: 785 | 807 bp |

| | | |
|---|---|---|
| DNA Sequence | ORF Start: at 1 | ORF Stop: end of sequence |

ACCGGATCCCTCACCTTGGAAGAGATTATTGGCATCGGGGGCTTTGGGAAGGTCTATCGTGCTTTCTG
GATAGGGGATGAGGTTGCTGTGAAAGCAGCTCGCCACGACCCTGATGAGGACATCAGCCAGACCATAG
AGAATGTTCGCCAAGAGGCCAAGCTCTTCGCCATGCTGAAGCACCCCAACATCATTGCCCTAAGAGGG
GTATGTCTGAAGGAGCCCAACCTCTGCTTGGTCATGGAGTTTGCTCGTGGAGGACCTTTGAATAGAGT
GTTATCTGGGAAAAGGATTCCCCCAGACATCCTGGTGAATTGGGCTGTGCAGATTGCCAGAGGGATGA
ACTACTTACATGATGAGGCAATTGTTCCCATCATCCACCGCGACCTTAAGTCCAGCAACATATTGATC
CTCCAGAAGGTGGAGAATGGAGACCTGAGCAACAAGATTCTGAAGATCACTGATTTTGGCCTGGCTCG
GGAATGGCACCGAACCACCAAGATGAGTGCGGCAGGGACGTATGCTTGGATGGCACCCGAAGTCATCC
GGGCCTCCATGTTTTCCAAAGGCAGTGATGTGTGGAGCTATGGGGTGCTACTTTGGGAGTTGCTGACT
GGTGAGGTGCCCTTTCGAGGCATTGATGGCTTAGCAGTCGCTTATGGAGTGGCCATGAACAAACTCGC
CCTTCCTATTCCTTCTACGTGCCCAGAACCTTTTGCCAAACTCATGGAAGACTGCTGGAATCCTGATC
CCCACTCACGACCATCTTTCACGAATATCCTGGACCAGCTAACCACCTACTCGAGGGC

NOV51b, 274082270

| | | | |
|---|---|---|---|
| Protein Sequence | SEQ ID NO: 786 | 269 aa | MW at 29861.3kD |

TGSLTLEEIIGIGGFGKVYRAFWIGDEVAVKAARHDPDEDISQTIENVRQEAKLFAMLKHPNIIALRG
VCLKEPNLCLVMEFARGGPLNRVLSGKRIPPDILVNWAVQIARGMNYLHDEAIVPIIHRDLKSSNILI
LQKVENGDLSNKILKITDFGLAREWHRTTKMSAAGTYAWMAPEVIRASMFSKGSDVWSYGVLLWELLT
GEVPFRGIDGLAVAYGVAMNKLALPIPSTCPEPFAKLMEDCWNPDPHSRPSFTNILDQLTTILEG

| | |
|---|---|
| SEQ ID NO: 787 | 3092 bp |

| | | |
|---|---|---|
| NOV51c, SNP13376041 of | ORF Start: ATG at 1 | ORF Stop: TAG at 3073 |

| | | |
|---|---|---|
| CG56071-01, DNA Sequence | SNP Pos: 2712 | SNP Change: T to C |

ATGGAGCCCTCCAGAGCGCTTCTCGGCTGCCTAGCGAGCGCCGCCGCTGCCGCCCCGCCGGAAGAGGA
TGGAGCAGGGGCCGGGGCCGAGGAGGAGGAGGAGGAGGAGGAGGAGGCGGCGGCGGCGGTGGGCCCCG
GGGAGCTGGGCTGCGACGCGCCGCTGCCCTACTGGACGGCCGTGTTCGAGTACGAGGCGGCGGGCGAG
GACGAGCTGACCCTGCGGCTGGGCGACGTGGTGGAGGTGCTGTCCAAGGACTCGCAGGTGTCCGGCGA
CGAGGGCTGGTGGACCGGGCAGCTGAACCAGCGGGTGGGCATCTTCCCCAGCAACTACGTGACCCCGC
GCAGCGCCTTCTCCAGCCGCTGCCAGCCCGGCGGCGAAATTGATTTTGCGGAGCTCACCTTGGAAGAG
ATTATTGGCATCGGGGGCTTTGGGAAGGTCTATCGTGCTTTCTGGATAGGGGATGAGGTTGCTGTGAA
AGCAGCTCGCCACGACCCTGATGAGGACATCAGCCAGACCATAGAGAATGTTCGCCAAGAGGCCAAGC
TCTTCGCCATGCTGAAGCACCCCAACATCATTGCCCTAAGAGGGGTATGTCTGAAGGAGCCCAACCTC
TGCTTGGTCATGGAGTTTGCTCGTGGAGGACCTTTGAATAGAGTGTTATCTGGGAAAAGGATTCCCCC
AGACATCCTGGTGAATTGGGCTGTGCAGATTGCCAGAGGGATGAACTACTTACATGATGAGGCAATTG
TTCCCATCATCCACCGCGACCTTAAGTCCAGCAACGTATTGATCCTCCAGAAGGTGGAGAATGGAGAC
CTGAGCAACAAGATTCTGAAGATCACTGATTTTGGCCTGGCTCGGGAATGGCACCGAACCACCAAGAT
GAGTGCGGCAGGGACGTATGCTTGGATGGCACCCGAAGTCATCCGGGCCTCCATGTTTTCCAAAGGCA
GTGATGTGTGGAGCTATGGGGTGCTACTTTGGGAGTTGCTGACTGGTGAGGTGCCCTTTCGAGGCATT
GATGGCTTAGCAGTCGCTTATGGAGTGGCCATGAACAAACTCGCCCTTCCTATTCCTTCTACGTGCCC
AGAACCTTTTGCCAAACTCATGGAAGACTGCTGGAATCCTGATCCCCACTCACGACCATCTTTCACGA
ATATCCTGGACCAGCTAACCACCATAGAGGAGTCTGGTTTCTTTGAAATGCCCAAGGACTCCTTCCAC
TGCCTGCAGGACAACTGGAAACACGAGATTCAGGAGATGTTTGACCAACTCAGGGCCAAAGAAAAGGA
ACTTCGCACCTGGGAGGAGGAGCTGACGCGGGCTGCACTGCAGCAGAAGAACCAGGAGGAACTGCTGC
GGCGTCGGGAGCAGGAGCTGGCCGAGCGGGAGATTGACATCCTGGAACGGGAGCTCAACATCATCATC
CACCAGCTGTGCCAGGAGAAGCCCCGGGTGAAGAAACGCAAGGGCAAGTTCAGGAAGAGCCGGCTGAA
GCTCAAGGATGGCAACCGCATCAGCCTCCCTTCTGGTTTCCAGCACAAGTTCACGGTGCAGGCCTCCC
CTACCATGGATAAAAGGAAGAGTCTTATCAACAGCCGCTCCAGTCCTCCTGCAAGCCCCACCATCATT
CCTCGCCTTCGAGCCATCCAGTGTGAGACTGTTTCCCAAATTAGCTGGGGCCAGAACACACAGGGGCA
CCTGTCCGAAAGCAGCAAAACCTGGGGCAGGAGCTCAGTCGTCCCAAAGGAGGAAGGGGAGGAGGAGG
AGAAGAGGGCCCCAAAGAAGAAGGGACGGACGTGGGGGCCAGGGACGCTTGGTCAGAAGGAGCTTGCC
TCGGGAGATGAACTCAAGTCCCTGGTAGATGGATATAAGCAGTGGTCGTCCAGTGCCCCCAACCTGGT
GAAGGGCCCAAGGAGTACCCCGGCCCTGCCAGGGTTCACCAGCCTTATGGAGATGGAGGATGAGGACA
GTGAAGGCCCAGGGAGTGGAGAGAGTCGCCTACAGCATTCACCCAGCCAGTCCTACCTCTGTATCCCA
TTCCCTCGTGGAGAGCCCACCCCAGTCAACTCGGCCACGAGTACCCCTCAGCTGACGCCAACCAACAG
CCTCAAGCGGGGCGGTGCCCACCACCGCCGCTGCGAGGTGGCTCTGCTCGGCTGTGGGGCTGTTCTGG
CAGCCACAGGCCTAGGGTTTGACTTGCTGGAAGCTGGCAAGTGCCAGCTGCTTCCCCTGGAGGAGCCT
GAGCCACCAGCCCGGGAGGAGAAGAAAAGACGGGAGGGTCTTTTTCAGAGGTCCAGCCGTCCTCGTCG
GAGCACCAGCCCCCCATCCCGAAAGCTTTTCAAGAAGGAGGAGCCCATGCTGTTGCTAGGAGACCCCT
CTGCCTCCCTGACGCTGCTCTCCCTCTCCTCCATCTCCGAGTGCAACTCCACACGCTCCCTGCTGCAG
TCCGACAGCGATGAAATTGTCGTGTATGAGATGCCAGTCAGCCCAGTCGAGGCCCCTCCCCTGAGTCC
ATGTACCCACAACCCCCTGGTCAATGTCCGAGTAGAGCGCTTCAAACGAGATCCTAACCAATCTCTGA
CTCCCACCCATGTCACCCTCACCACCCCCTCGCAGCCCAGCAGTCACCGGCGGACTCCTGCTGATGGG
GCCCTTAAGCCAGAGACTCTCCTAGCCAGCAGGAGCCCCAGTCCCAGCCGAGACCCAGGCGAATTCCC
CCGTCTCCCTGACCCCAATGTGGTCTTCCCCCCAACCCCAAGGCGCTGGAACACTCAGCAGGACTCTA
CCTTGGAGAGACCCAAGACTCTGGAGTTTCTGCCTCGGCCGCGTCCTTCTGCCAACCGGCAACGGCTG
GACCCTTGGTGGTTTGTGTCCCCCAGCCATGCCCGCAGCACCTCCCCAGCCAACAGCTCCAGCACAGA
GACGCCCGGGCCGCTGCCCCCGACTGAGCGGACGCTCCTGGACCTGGATGCAGAGGGGCAGAGTCAGG
ACAGCACCGTGCCGCTGTGCAGAGCGGAACTGAACACACACAGGCCTGCCCCTTATGAGATCCAGCAG
GAGTTCTGGTCTTAGCACGAAAAGGATTGGGG

| | | |
|---|---|---|
| NOV51c, SNP13376041 of | SEQ ID NO: 788 | MW at 113682.1kD |

| | | | |
|---|---|---|---|
| CG56071-01, Protein Sequence | SNP Pos: 904 | 1024 aa | SNP Change: Gly to Gly |

MEPSRALLGCLASAAAAAPPGEDGAGAGAEEEEEEEEEAAAAVGPGELGCDAPLPYWTAVFEYEAAGE
DELTLRLGDVVEVLSKDSQVSGDEGWWTGQLNQRVGIFPSNYVTPRSAFSSRCQPGGEIDFAELTLEE
IIGIGGFGKVYRAFWIGDEVAVKAARHDPDEDISQTIENVRQEAKLFAMLKHPNIIALRGVCLKEPNL
CLVMEFARGGPLNRVLSGKRIPPDILVNWAVQIARGMNYLHDEAIVPIIHRDLKSSNVLILQKVENGD
LSNKILKITDFGLAREWHRTTKMSAAGTYAWMAPEVIRASMFSKGSDVWSYGVLLWELLTGEVPFRGI
DGLAVAYGVANNKLALPIPSTCPEPFAKLMEDCWNPDPHSRPSFTNILDQLTTIEESGFFEMPKDSFH
CLQDNWKHEIQEMFDQLRAKEKELRTWEEELTRAALQOKNQEELLRRREQELAEREIDILERELNIII
HQLCQEKPRVKKRKGKFRKSRLKLKDGNRISLPSGFQHKFTVQASPTMDKRKSLINSRSSPPASPTII
PRLRAIQCETVSQISWGQNTQGHLSESSKTWGRSSVVPKEEGEEEEKRAPKKKGRTWGPGTLGQKELA
SGDELKSLVDGYKQWSSSAPNLVKGPRSTPALPGFTSLMEMEDEDSEGPGSGESRLQHSPSQSYLCIP
FPRGEPTPVNSATSTPQLTPTNSLKRGGAHHRRCEVALLGCGAVLAATGLGFDLLEAGKCQLLPLEEP
EPPAREEKKRREGLFQRSSRPRRSTSPPSRKLFKKEEPMLLLGDPSASLTLLSLSSISECNSTRSLLQ
SDSDEIVVYEMPVSPVEAPPLSPCTHNPLVNVRVERFKRDPNQSLTPTHVTLTTPSQPSSHRRTPSDG
ALKPETLLASRSPSPSRDPGEFPRLPDPNVVFPPTPRRWNTQQDSTLERPKTLEFLPRPRPSANRQRL
DPWWFVSPSHARSTSPANSSSTETPGPLPPTERTLLDLDAEGQSQDSTVPLCRAELNTHRPAPYEIQQ
EFWS

A ClustalW comparison of the above protein sequences yields the following sequence alignment shown in Table 51B.

TABLE 51B
Comparison of the NOV51 protein sequences.

| | |
|---|---|
| NOV51a | MEPSRALLGCLASAAAAAPPGEDGAGAGAEEEEEEEEEAAAAVGPGELGCDAPLPYWTAV |
| NOV51b | ------------------------------------------------------------ |
| NOV51a | FEYEAAGEDELTLRLGDVVEVLSKDSQVSGDEGWWTGQLNQRVGIFPSNYVTPRSAFSSR |
| NOV51b | ------------------------------------------------------------ |
| NOV51a | CQPGGEIDFAELTLEEIIGIGGFGKVYRAFWIGDEVAVKAARHDPDEDISQTIENVRQEA |
| NOV51b | --------TGSLTLEEIIGIGGFGKVYRAFWIGDEVAVKAARHDPDEDISQTIENVRQEA |
| NOV51a | KLFANLKHPNIIALRGVCLKEPNLCLVMEFARGGPLNRVLSGKRIPPDILVNWAVQIARG |
| NOV51b | KLFANLKHPNIIALRGVCLKEPNLCLVMEFARGGPLNRVLSGKRIPPDILVNWAVQIARG |
| NOV51a | MNYLHDEAIVPIIHRDLKSSNVLILQKVENGDLSNKILKITDFGLAREWHRTTKMSAAGT |
| NOV51b | MNYLHDEAIVPIIHRDLKSSNILILQKVENGDLSNKILKITDFGLAREWHRTTKMSAAGT |
| NOV51a | YAWMAPEVIRASMFSKGSDVWSYGVLLWELLTGEVPFRGIDGLAVAYGVAMNKLALPIPS |
| NOV51b | YAWMAPEVIRASMFSKGSDVWSYGVLLWELLTGEVPFRGIDGLaVAYGVAMNKLALPIPS |
| NOV51a | TCPEPFAKLMEDCWNPDPHSRPSFTNILDQLTTIEESGFFEMPKDSFHCLQDNWKHEIQE |
| NOV51b | TCPEPFAKLMEDCWNPDPHSRPSFTNILDQLTTILEG----------------------- |
| NOV51a | MFDQLRAKEKELRTWEEELTRAALQQKNQEELLRRREQELAEREIDILERELNIIIHQLC |
| NOV51b | ------------------------------------------------------------ |
| NOV51a | QEKPRVKKRKGKFRKSRLKLKDGNRISLPSGFQHKFTVQASPTMDKRKSLINSRSSPPAS |
| NOV51b | ------------------------------------------------------------ |
| NOV51a | PTIIPRLRAIQCETVSQISWGQNTQGHLSESSKTWGRSSVVPKEEGEEEEKRAPKKKGRT |
| NOV51b | ------------------------------------------------------------ |
| NOV51a | WGPGTLGQKELASGDELKSLVDGYKQWSSSAPNLVKGPRSTPALPGFTSLMEMEDEDSEG |
| NOV51b | ------------------------------------------------------------ |
| NOV51a | PGSGESRLQHSPSQSYLCIPFPRGEPTPVNSATSTPQLTPTNSLKRGGAHHRRCEVALLG |
| NOV51b | ------------------------------------------------------------ |
| NOV51a | CGAVLAATGLGFDLLEAGKCQLLPLEEPEPPAREEKKRREGLFQRSSRPRRSTSPPSRKL |
| NOV51b | ------------------------------------------------------------ |
| NOV51a | FKKEEPMLLLGDPSASLTLLSLSSISECNSTRSLLQSDSDEIVVYEMPVSPVEAPPLSPC |
| NOV51b | ------------------------------------------------------------ |
| NOV51a | THNPLVNRVERFKRDPNQSLTPTHVTLTTPSQPSSHRRTPSDGALKPETLLASSRSPSPS |
| NOV51b | ------------------------------------------------------------ |
| NOV51a | RDPGEFPRLPDPNVVFPPTPRRWNTQQDSTLERPKTLEFLPRPRPSANRQRLDPWWFVSP |
| NOV51b | ------------------------------------------------------------ |
| NOV51a | SHARSTSPANSSSTETPGPLPPTERTLLDLDAEGQSQDSTVPLCRAELNTHRPAPYEIQQ |
| NOV51b | ------------------------------------------------------------ |
| NOV51a | EFWS |
| NOV51b | ---- |
| NOV51a | (SEQ ID NO: 784) |
| NOV51b | (SEQ ID NO: 786) |

Further analysis of the NOV51a protein yielded the following properties shown in Table 51C.

TABLE 51C
Protein Sequence Properties NOV51a

| | |
|---|---|
| SignalP analysis: | Cleavage site between residues 18 and 19 |

PSORT II analysis:

| | |
|---|---|
| PSG: | a new signal peptide prediction method |
| | N-region: length 5; pos.chg 1; neg.chg 1 |
| | H-region: length 16; peak value 7.95 |
| | PSG score: 3.55 |
| GvH: | von.Heijne's method for signal seq. recognition |
| | GvH score (threshold: −2.1): 0.45 |
| | possible cleavage site: between 17 and 18 |

>>> Seems to have a cleavable signal peptide (1 to 17)

| | |
|---|---|
| ALOM: | Klein et al's method for TM region allocation |
| | Init position for calculation: 18 |
| | Tentative number of TMS(s) for the threshold 0.5: 1 |
| | Number of TMS(s) for threshold 0.5: 1 |
| | INTEGRAL Likelihood = −3.66 Transmembrane 716 - 732 |
| | PERIPHERAL Likelihood = 2.44 (at 342) |
| | ALOM score: −3.66 (number of TMSs: 1) |
| MTOP: | Prediction of membrane topology (Hartmann et al.) |
| | Center position for calculation: 8 |
| | Charge difference: −10.0 C(−9.0) - N( 1.0) |
| | N >= C: N-terminal side will be inside |

>>>membrane topology: type 1a (cytoplasmic tail 733 to 1024)
MITDISC: discrimination of mitochondrial targeting seq

| | | | |
|---|---|---|---|
| R content: | 1 | Hyd Moment (75): | 4.60 |
| Hyd Moment (95): | 9.12 | G content: | 2 |
| D/E content: | 2 | S/T content: | 2 |
| Score: −6.34 | | | |

Gavel: prediction of cleavage sites for mitochondrial preseq

R-2 motif at 15 SRA|LL

NUCDISC: discrimination of nuclear localization signals

pat4: KKRK (5) at 487
pat4: PKKK (4) at 594
pat4: KKRR (5) at 756
pat4: RPRR (4) at 768
pat7: PRVKKRK (5) at 484
pat7: PTMDKRK (3) at 522
pat7: PKKKGRT (5) at 594
bipartite: KKRREGLFQRSSRPRRS at 756
content of basic residues: 12.1%
NLS Score: 2.27

KDEL: ER retention motif in the C-terminus: none
ER Membrane Retention Signals:

XXRR-like motif in the N-terminus: EPSR

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 734
LL at 742
LL at 788
LL at 789
LL at 799
LL at 814
LL at 891

checking 63 PROSITE DNA binding motifs: none
checking 71 PROSITE ribosomal protein motifs: none
checking 33 PROSITE prokaryotic DNA binding motifs: none
NNCN: Reinhardt≡s method for Cytoplasmic/Nuclear discrimination

Prediction: nuclear
Reliability: 89

```
COIL: Lupas's algorithm to detect coiled-coil regions
  409 L 0.51
  410 Q 0.87
  411 D 0.99
  412 N 0.99
  413 W 0.99
  414 K 1.00
  415 H 1.00
  416 E 1.00
  417 I 1.00
  418 Q 1.00
  419 E 1.00
  420 M 1.00
  421 F 1.00
  422 D 1.00
  423 Q 1.00
  424 L 1.00
  425 R 1.00
  426 A 1.00
  427 K 1.00
  428 E 1.00
  429 K 1.00
  430 E 1.00
  431 L 1.00
  432 R 1.00
  433 T 1.00
  434 W 1.00
  435 E 1.00
  436 E 1.00
  437 E 1.00
  438 L 1.00
  439 T 1.00
  440 R 1.00
  441 A 1.00
  442 A 0.97
  443 L 0.97
  444 0 0.96
  445 Q 0.96
  446 K 0.96
  447 N 0.96
  448 0 0.96
  449 E 0.96
  450 E 0.96
  451 L 0.81
  452 L 0.81
  453 R 0.81
  454 R 0.81
  455 R 0.71
  456 E 0.71
  457 0 0.71
  458 E 0.71
  459 L 0.71
  460 A 0.71
  461 E 0.71
  462 R 0.64
  463 E 0.64
  464 I 0.64
  465 D 0.64
  466 I 0.64
  467 L 0.64
  468 E 0.64
  469 R 0.64
  470 E 0.64
  471 L 0.64
  472 N 0.64
  473 I 0.64
  474 I 0.64
  475 I 0.64
  476 H 0.64
  477 0 0.64
  478 L 0.64
  479 C 0.64
  480 Q 0.64
  481 E 0.64
  482 K 0.64
```

total: 74 residues

--------------------------
Final Results (k = 9/23):

44.4%: extracellular, including cell wall
22.2%: Golgi
22.2%: endoplasmic reticulum
11.1%: plasma membrane

>> prediction for CG56071-01 is exc (k = 9)

A search of the NOV51a protein against the Geneseq database, a proprietary database that contains sequences published in patents and patent publication, yielded several homologous proteins shown in Table 51D.

TABLE 51a
Geneseq Results for NOV51a

| | | NOV51a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB98408 | Human NOV7, a mixed lineage | 1..1024 | 1024/1024 (100%) | 0.0 |
| | kinase 2-like protein - Homo | 1..1024 | 1024/1024 (100%) | |
| | sapiens, 1024 aa. | | | |
| | [WO200255704-A2, 18-Jul.- | | | |
| | 2002] | | | |
| AAE21717 | Human PKIN-12 protein- | 1..1024 | 1003/1111 (90%) | 0.0 |
| | Homo sapiens, 1097 aa. | 1..1097 | 1006/1111(90%) | |
| | [WO200218557-A2, 07-Mar.- | | | |
| | 2002] | | | |
| AAE11775 | Human kinase (PKJN)-9 protein | 1..1024 | 994/1072 (92%) | 0.0 |
| | - Homo sapiens, 1046 aa. | 1..1046 | 997/1072 (92%) | |
| | [WO200181555-A2, 01-Nov.- | | | |
| | 2001] | | | |
| ABP61000 | Novel human protein. SEQ ID | 24..1024 | 540/1061 (50%) | 0.0 |
| | 87 - Homo sapiens, 1021 aa. | 19..1021 | 682/1061 (63%) | |
| | [WO200250105-A1, 27-Jun.- | | | |
| | 2002] | | | |
| ABB80923 | Novel human protein (NHP) | 24.. 1024 | 537/1076 (49%) | 0.0 |
| | kinase - Homo sapiens, 1036 aa. | 19..1036 | 684/1076 (62%) | |
| | [WO200255685-A2, 18-Jul.- | | | |
| | 2002] | | | |

In a BLAST search of public sequence databases, the NOV51a protein was found to have homology to the proteins shown in the BLASTP data in Table 51E.

TABLE 51E
Public BLASTP Results for NOV51a

| | | NOV51a | Identities/ | |
|---|---|---|---|---|
| Protein | | Residues/ | Similarities for | Ex- |
| Accession | Protein/ | Match | the Matched | pect |
| Number | /Organism/Length | Residues | Portion | Value |
| Q9H2N5 | Mixed lineage kinase | 53..1024 | 952/1080 (88%) | 0.0 |
| | MLK1 - Homo sapiens | 1..1066 | 955/1080 (88%) | |
| | (Human), 1066 aa | | | |
| | (fragment). | | | |
| Q8K2L2 | Similar to mitogen- | 341..1024 | 634/746 (84%) | 0.0 |
| | activated protein | 1..732 | 649/746 (86%) | |
| | kinase kinase 9 - | | | |
| | Mus musculus | | | |
| | (Mouse), 732 aa | | | |
| | (fragment). | | | |
| Q8BIG8 | Mixed lineage kinase | 1..616 | 572/628 (91%) | 0.0 |
| | MLKI homolog - Mus | 1..607 | 581/628 (92%) | |
| | musculus (Mouse), | | | |
| | 608 aa. | | | |
| Q02779 | Mitogen-activated | 35..1006 | 579/988 (58%) | 0.0 |
| | protein kinase | 2..950 | 699/988 (70%) | |
| | kinase kinase 10 | | | |
| | (EC 2.7.1.37) (Mixed | | | |
| | lineage kinase 2) | | | |
| | (Protein kinase | | | |
| | MST) - Homo sapiens | | | |
| | (Human), 954 aa. | | | |
| Q8WWN1 | Mixed lineage | 24..1024 | 537/1076 (49%) | 0.0 |
| | kinase 4beta - | 19..1036 | 680/1076 (62%) | |
| | Homo sapiens | | | |
| | (Human), 1036 aa. | | | |

PFam analysis indicates that the NOV51a protein contains the domains shown in the Table 51F.

TABLE 51F
Domain Analysis of NOV51a

| | | Identities/ | |
|---|---|---|---|
| | NOV51a | Similarities for | |
| Pfam Domain | Match Region | the Matched Region | Expect Value |
| SH3 | 55..114 | 26/63 (41%) | 4.7e-15 |
| | | 50/63 (79%) | |
| pkinase | 132..393 | 100/307 (33%) | 7e-95 |
| | | 216/307 (70%) | |

### Example 52

The NOV52 clone was analyzed, and the nucleotide and encoded polypeptide sequences are shown in Table 52A.

TABLE 52A

| NOV52 Sequence Analysis | | |
|---|---|---|
| NOV52a, CG56142-01 | SEQ ID NO: 789 | 866 bp |

| | | |
|---|---|---|
| DNA Sequence | ORF Start: ATG at 19 | ORF Stop: TGA at 820 |

CCCAGCCTTGAAGACAGAATGAGAGGGGTTTCCTGTCTCCAGGTCCTGCTCCTTCTGGTGCTGGCCTG
CGGGCAGCCCCGCATGTCCAGTCGGATCGTTGGGGGCCGGGATGGCCGGGACGGAGAGTGGCCGTGGC
AGGCGAGCATCCAGCATCGTGGGGCACACGTGTGCGGGGGGTCGCTCATCGCCCCCCAGTGGGTGCTG
ACAGCGGCGCACTGCTTCCCCAGGGCACTGCCAGCTGAGTACCGCGTGCGCCTGGGGGCGCTGCGTCT
GGGCTCCACCTCGCCCCGCACGCTCTCGGTGCCCGTGCGACGGGTGCCGCTGCCCCCGGACTACTCCG
AGGACGGGGCCCGCGGCGACCTGGCACTGCTGCAGCTGCGTCGCCCGGTGCCCCTGAGCGCTCGCGTC
CAACCCGTCTGCCTGCCCGTGCCCGGCGCCCGCCCGCCGCCCGGCACACCATGCCGGGTCACCGGCTG
GGGCAGCCTCCGCCCAGGAGTGCCCCTCCCAGAGTGGCGACCGCTACAAGGAGTAAGGGTGCCGCTGC
TGGACTCGCGCACCTGCGACGGCCTCTACCACGTGGGCGCGGACGTGCCCCAGGCTGAGCGCATTGTG
CTGCCTGGGAGTCTGTGTGCCGGCTACCCCCAGGGCCACAAGGACGCCTGCCAGGTGTGCACCCAGCC
TCCCCAGCCTCCGGAGTCCCCTCCCTGTGCCCAGCACCCTCCCTCCCTGAACTCCAGGACCCAGGACA
TCCCAACTCAGGCTCAGGATCCTGGCCTCCAACCTAGAGGCACCACGCCAGGGGTCTGGAACCCTGAG
AACTGAAGTCCTGGGAGGGCTGGGACTTAGGCTCCTCTTTCTCCTGCAGG

NOV52a, CG56142-01

| | | | |
|---|---|---|---|
| Protein Sequence | SEQ ID NO: 790 | 267 aa | MW at 28699.8kD |

MRGVSCLQVLLLLVLACGQPRNSSRIVGGRDGRDGEWPWQASEQHRGAHVCGGSLIAPQWVLTAAHCF
PRALPAEYRVRLGALRLGSTSPRTLSVPVRRVLLPPDYSEDGARGDLALLQLRRPVPLSARVQPVCLP
VPGARPPPGTPCRVTGWGSLRPGVPLPEWRPLQGVRVPLLDSRTCDGLYHVGADVPQAERIVLPGSLC
AGYPQGHKDACQVCTQPPQPPESPPCAQHPPSLNSRTQDIPTQAQDPGLQPRGTTPGVWNPEN

| | | |
|---|---|---|
| NOV52b, CG56142-04 | SEQ ID NO: 791 | 638 bp |

| | | |
|---|---|---|
| DNA Sequence | ORF Start: ATG at 14 | ORF Stop: TGA at 578 |

CCTTGAAGACAGAATGAGAGGGGTTTCCTGTCTCCAGGTCCTGCTCCTTCTGGTGCTGGGAGCTGCTG
GGACTCAGGGAAGGAAGTCTGCAGCCTGCGGGCAGCCCCGCATGTCCAGTCGGATCGTTGGGGGCCGG
GATGGCCGGGACGGAGAGTGGCCGTGGCAGGCGAGCATCCAGCATCGTGGGGCACACGTGTGCGGGGG
GTCGCTCATCGCCCCCCAGTGGGTGCTGACAGCGGCGCACTGCTTCCCCAGTGCCCCTCCAGAGTGGC
GACCGCTACAAGGAGTAAGGGTGCCGCTGCTGGACTCGCGCACCTGCGACGGCCTCTACCACGTGGGC
GCGGACGTGCCCCAGGCTGAGCGCATTGTGCTGCCTGGGAGTCTGTGTGCCGGCTACCCCCAGGGCCA
CAAGGACGCCTGCCAGGTGTGCACCCAGCCTCCCCAGCCTCCGGAGTCCCCTCCCTGTGCCCAGCACC
CTCCCTCCCTGAACTCCAGGACCCAGGACATCCCAACTCAGGCTCAGGATCCTGGCCTCCAACCTAGA
GGCACCACGCCAGGGGTCTGGAACCCTGAGAACTGAAGTCCTGGGAGGGCTGGGACTTAGGCTCCTCT
TTCTCCTGGAGGGTGATTCTGGGGGA

NOV52b, CG56142-04

| | | | |
|---|---|---|---|
| Protein Sequence | SEQ ID NO: 792 | 188 aa | MW at 19994.5kD |

MRGVSCLQVLLLLVLGAAGTQGRKSAACGQPRMSSRIVGGRDGRDGEWPWQASIQHRGAHVCGGSLIA
PQWVLTAAHCFPSAPPEWRPLQGVRVPLLDSRTCDGLYHVGADVPQAERIVLPGSLCAGYPQGHKDAC
QVCTQPPQPPESPPCAQHPPSLNSRTQDIPTQAQDPGLQPRGTTPGVWNPEN

| | | |
|---|---|---|
| NOV52c, 276873337 | SEQ ID NO: 793 | 1165 bp |

| | | |
|---|---|---|
| DNA Sequence | ORF Start: at 606 | ORF Stop: TGA at 948 |

CACCAAGCTTTGGGACTACGAGACGCCCAAGGTGATCGTGGTGAGGAACCGGCGCCTGGGGGTCCTGT
ACCGCGCCGTGCAGCTGCTCATCCTGCTCTACTTCGTGTGGTACGTATTCATCGTGCAGAAAAGCTAC
CAGGAGAGCGAGACGGGCCCCGAGAGCTCCATCATCACCAAGGTCAAGGGGATCACCACGTCCGAGCA
CAAAGTGTGGGACGTGGAGGAGTACGTGAAGCCCCCCGAGGGGGGCAGCGTGTTCAGCATCATCACCA
GGGTCGAGGCCACCCACTCCCAGACCCAGGGAACCTGCCCCGAGAGCATAAGGGTCCACAACGCCACC
TGCCTCTCCGACGCCGACTGCGTGGCTGGGGAGCTGGACATGCTGGGAAACGGCCTGAGGACCGGGCG
CTGTGTGCCCTATTACCAGGGGCCCTCCAAGACCTGCGAGGTGTTCGGCTGGTGCCCGGTGGAAGATG
GGGCCTCTGTCAGCGAATTTCTGGGTACGATGGCCCCAAATTTCACCATCCTCATCAAGAACAGCATC
CACTACCCCAAATTCCACTTCTCCAAGGGCAACATCGCCGACCGCACAGACGGGTACCTGAAGCGCTG
CACGTTCCACGAGGCCTCCGACCTCTACTGCCCCATCTTCAAGCTGGGCTTTATCGTGGAGAAGGCTG
GGGAGAGCTTCACAGAGCTCGCACACAAGGGTGGTGTCATCGGGGTCATTATCAACTGGGACTGTGAC
CTGGACCTGCCTGCATCGGAGTGCAACCCCAAGTACTCCTTCCGGAGGCTTGACCCCAAGCACGTGCC
TGCCTCGTCAGGCTACAACTTCAGGTTTGCCAAATACTACAAGATCAATGGCACCACCACCCGCACGC
TCATCAAGGCCTACGGGATCCACATTGACGTCATTGTGCATGGACAGGTCGGGAAGTTCAGCCTGATT
CCCACCATTATTAATCTGGCCACAGCTCTGACTTCCGTCGGGGTGGTAAGGAACCCTCTCTGGGGTCC
CAGCGGGTGCGGGGGGTCCACCAGGCCCTTACACACCGGTCTCTGCTGGCCCCAGGGCTCCTTCCTGT
GCGACTGGATCTTGCTAACATTCATGAACAAAAACAAGGTCTACAGCCATAAGAAATTTGACAAGGTG
GTCGACGGC

NOV52c, 276873337

| | | | |
|---|---|---|---|
| Protein Sequence | SEQ ID NO: 794 | 114 aa | MW at 11702.8kD |

SAARSTRPPTSTAPSSSWALSWRRLGRASQSSHTRVVSSGSLSTGTVTWTCLHRSATPSTPSGGLTPS
TCLPRQATTSGLPNTTRSMAPPPARSSRPTGSTLTSLCMDRSGSSA

| | | |
|---|---|---|
| NOV52d, 276863970 | SEQ ID NO: 795 | 1164 bp |

| | | |
|---|---|---|
| DNA Sequence | ORF Start: at 605 | ORF Stop: TGA at 947 |

CACAAGCTTTGGGACTACGAGACGCCCAAGGTGATCGTGGTGAGGAACCGGCGCCTGGGGGTCCTGTA
CCGCGCCGTGCAGCTGCTCATCCTGCTCTACTTCGTGTGGTACGTATTCATCGTGCAGAAAAGCTACC
AGGAGAGCGAGACGGGCCCCGAGAGCTCCATCATCACCAAGGTCAAGGGGATCACCACGTCCGAGCAC
AAAGTGTGGGACGTGGAGGAGTACGTGAAGCCCCCCGAGGGGGGCAGCGTGTTCAGCATCATCACAGG
GGTCGAGGCCACCCACTCCCAGACCCAGGGAACCTGCCCCGAGAGCATAAGGGTCCACAACGCCACCT
GCCTCTCCGACGCCGACTGCGTGGCTGGGGAGCTGGACATGCTGGGAAACGGCCTGAGGACCGGGCGC
TGTGTGCCCTATTACCAGGGGCCCTCCAAGACCTGCGAGGTGTTCGGCTGGTGCCCGGTGGAAGATGG
GGCCTCTGTCAGCCAATTTCTGGGTACGATGGCCCCAAATTTCACCATCCTCATCAAGAACAGCATCC
ACTACCCCAAATTCCACTTCTCCAAGGGCAACATCGCCGACCGCACAGACGGGTACCTGAAGCGCTGC
ACGTTCCACGAGGCCTCCGACCTCTACTGCCCCATCTTCAAGCTGGGCTTTATCGTGGAGAAGGCTGG
GGAGAGCTTCACAGAGCTCGCACACAAGGGTGGTGTCATCGGGGTCATTATCAACTGGGACTGTGACC
TGGACCTGCCTGCATCGGAGTGCAACCCCAAGTACTCCTTCCGGAGGCTTGACCCCAAGCACGTGCCT
GCCTCGTCAGGCTACAACTTCAGGTTTGCCAAATACTACAAGATCAATGGCACCACCACCCGCACGCT
CATCAAGGCCTACGGGATCCGCATTGACGTCATTGTGCATGGACAGGTCGGGAAGTTCAGCCTGATTC
CCACCATTATTAATCTGGCCACAGCTCTGACTTCCGTCGGGGTGGTAAGGAACCCTCTCTGGGGTCCC
AGCGGGTGCGGGGGGTCCACCAGGCCCTTACACACCGGTCTCTGCTGGCCCCAGGGCTCCTTCCTGTG
CGACTGGATCTTGCTAACATTCATGAACAAAAACAAGGTCTACAGCCATAAGAAATTTGACAAGGTGG
TCGACGGC

NOV52d, 276863970

| | | | |
|---|---|---|---|
| Protein Sequence | SEQ ID NO: 796 | 114 aa | MW at 11672.8kD |

SARSTRPPTSTAPSSSWALSWRRLGRASQSSHTRWSSGSLSTGTVTWTCLHRSATPSTPSGGLTPS
TCLPRQATTSGLPNTTRSMAPPPARSSRPTGSALTSLCMDRSGSSA

| | | |
|---|---|---|
| NOV52e, 276863992 | SEQ ID NO: 797 | 1087 bp |
| DNA Sequence | ORF Start: at 606 | ORF Stop: TGA at 948 |

CACCAAGCTTTGGGACTACGAGACGCCCAAGGTGATCGTGGTGAGGAACCGGCGCCTGGGGGTCCTGT
ACCGCGCCGTGCAGCTGCTCATCCTGCTCTACTTCGTGTGGTACGTATTCATCGTGCAGAAAAGCTAC
CAGGAGAGCGAGACGGGCCCCGAGAGCTCCATCATCACCAAGGTCAAGGGGATCACCACGTCCGAGCA
CAAAGTGTGGGACGTGGAGGAGTACGTGAAGCCCCCCGAGGGGGGCAGCGTGTTCAGCATCATCACCA
GGGTCGAGGCCACCCACTCCCAGACCCAGGGAACCTGCCCCGAGAGCATAAGGGTCCACAACGCCACC
TGCCTCTCCGACGCCGACTGCGTGGCTGGGGAGCTGGACATGCTGGGAAACGGCCTGAGGACCGGGCG
CTGTGTGCCCTATTACCAGGGGCCCTCCAAGACCTGCGAGGTGTTCGGCTGGTGCCCGGTGGAAGATG
GGGCCTCTGTCAGCCAATTTCTGGGTACGATGGCCCCAAATTTCACCATCCTCATCAAGAACAGCATC
CACTACCCCAAATTCCACTTCTCCAAGGGCAACATCGCCGACCGCACAGACGGGTACCTGAAGCGCTG
CACGTTCCACGAGGCCTCCGACCTCTACTGCCCCATCTTCAAGCTGGGCTTTATCGTGGAGAAGGCTG
GGGAGAGCTTCACAGAGCTCGCACAAGAGGGTGGTGTCATCGGGGTCATTATCAACTGGGACTGTGAC
CTGGACCTGCCTGCATCGGAGTGCAACCCCAAGTACTCCTTCCGGAGGCTTGACCCCAAGCACGTGCC
TGCCTCGTCAGGCTACAACTTCAGGTTTGCCAAATACTACAAGATCAATGGCACCACCACCCGCACGC
TCATCAAGGCCTACGGGATCCGCATTGACGTCATTGTGCATGGACAGGCCGGGAAGTTCAGCCTGATT
CCCACCATTATTAATCTGGCCACAGCTCTGACTTCCGTCGGGGTGGGCTCCTTCCTGTGCGACTGGAT
CTTGCTAACATTCATGAACAAAAACAAGGTCTACAGCCATAAGAAATTTGACAAGGTGGTCGACGGC

NOV52e, 276863992

| | | | |
|---|---|---|---|
| Protein Sequence | SEQ ID NO: 798 | 114 aa | MW at 11682.8kD |

SAARSTRPPTSTAPSSSWALSWRRLGRASQSSHTRVVSSGSLSTGTVTWTCLHRSATPSTPSGGLTPS
TCLPRQATTSGLPNTTRSMAPPPARSSRPTGSALTSLCMDRPGSSA

| | | |
|---|---|---|
| NOV52f, 2768733301 | SEQ ID NO: 799 | 1087 bp |

| | | |
|---|---|---|
| DNA Sequence | ORF Start: at 606 | ORF Stop: TGA at 948 |

CACCAAGCTTTGGGACTACGAGACGCCCAAGGTGATCGTGGTGAGGAACCGGCGCCTGGGGGTCCTGT
ACCGCGCCGTGCAGCTGCTCATCCTGCTCTACTTCGTGTGGTACGTATTCATCGTGCAGAAAAGCTAC
CAGGAGAGCGAGACGGGCCCCGAGAGCTCCATCATCACCAAGGTCAAGGGGATCACCACGTCCGAGCA
CAAAGTGTGGGACGTGGAGGAGTACGTGAAGCCCCCCGAGGGGGGCAGCGTGTTCAGCATCATCACCA
GGGTCGAGGCCACCCACTCCCAGACCCAGGGAACCTGCCCCGAGAGCATAAGGGTCCACAACGCCACC
TGCCTCTCCGACGCCGACTGCGTGGCTGGGGAGCTGGACATGCTGGGAAACGGCCTGAGGACCGGGCG
CTGTGTGCCCTATTACCAGGGGCCCTCCAAGACCTGCGAGGTGTTCGGCTGGTGCCCGGTGGAAGATG
GGGCCTCTGTCAGCCAATTTCTGGGTACGATGGCCCCAAATTTCACCATCCTCATCAAGAACAGCATC
CACTACCCCAAATTCCACTTCTCCAAGGGCAACATCGCCGACCGCACAGACGGGTACCTGAAGCGCTG
CACGTTCCACGAGGCCTCCGACCTCTACTGCCCCATCTTCAAGCTGGGCTTTATCGTGGAGAAGGCTG
GGGAGAGCTTCACAGAGCTCGCACACAAGGGTGGTGTCATCGGGGTCATTATCAACTGGGACTGTGAC
CTGGACCTGCCTGCATCGGAGTGCAACCCCAAGTACTCCTTCCGGAGGCTTGACCCCAAGCACGTGCC
TGCCTCGTCAGGCTACAACTTCAGGTTTGCCAAATACTACAAGATCAATGGCACCACCACCCGCACGC
TCATCAAGGCCTACGGGATCCGCATTGACGTCATTGTGCATGGACAGGCCGGGAAGTTCATCCTGATT
CCCACCATTATTAATCTGGCCACAGCTCTGACTTCCGTCGGGGTGGGCTCCTTCCTGTGCGACTGGAT
CTTGCTAACATTCATGAACAAAAACAAGGTCTACAGCCATAAGAAATTTGACAAGGTGGTCGACGGC

NOV52f, 276873330

| | | | |
|---|---|---|---|
| Protein Sequence | SEQ ID NO: 800 | 114 aa | MW at 11698.8kD |

SAARSTRPPTSTAPSSSWALSWRRLGRASQSSHTRVVSSGSLSTGTVTWTCLHRSATPSTPSGGLTPS
TCLPRQATTSGLPNTTRSMAPPPARSSRPTGSALTSLCMDRPGSSS

| | | |
|---|---|---|
| NOV52g, CG56142-02 | SEQ ID NO: 801 | 1020 bp |

| | | |
|---|---|---|
| DNA Sequence | ORF Start: ATG at 91 | ORF Stop: TAA at 931 |

AGGACTCTCCTCTCTTCTCCCTGCTGGCTCCAGACCAGAGTCCAAGCCCTAGGCAGTGCCACCCTTAC
CCAGCCCAGCCTTGAAGACAGAATGAGAGGGGTTTCCTGTCTCCAGGTCCTGCTCCTTCTGGTGCTGG
GAGCTGCTGGGACTCAGGGAAGGAAGTCTGCAGCCTGCGGGCAGCCCCGCATGTCCAGTCGGATCGTT
GGGGGCCGGGATGGCCGGGACGGAGAGTGGCCGTGGCAGGCGAGCATCCAGCATCGTGGGGCACACGT
GTGCGGGGGGTCGCTCATCGCCCCCCAGTGGGTGCTGACAGCGGCGCACTGCTTCCCCAGGAGGGCAC
TGCCAGCTGAGTACCGCGTGCGCCTGGGGGCGCTGCGTCTGGGCTCCACCTCGCCCCGCACGCTCTCG
GTGCCCGTGCGACGGGTGCTGCTGCCCCCGGACTACTCCGAGGACGGGGCCCGCGGCGACCTGGCACT
GCTGCAGCTGCGTCGCCCGGTGCCCCTGAGCGCTCGCGTCCAACCCGTCTGCCTGCCCGTGCCCGGCG
CCCGCCCGCCGCCCGGCACACCATGCCGGGTCACCGGCTGGGGCAGCCTCCGCCCAGGAGTGCCCCTC
CCAGAGTGGCGACCGCTACAAGGAGTAAGGGTGCCGCTGCTGGACTCGCGCACCTGCGACGGCCTCTA
CCACGTGGGCGCGGACGTGCCCCAGGCTGAGCGCATTGTGCTGCCTGGGAGTCTGTGTGCCGGCTACC
CCCAGGGCCACAAGGACGCCTGCCAGGGTGATTCTGGGGGACCTCTGACCTGCCTGCAGTCTGGGAGC
TGGGTCCTGGTGGGCGTGGTGAGCTGGGGCAAGGGTTGTGCCCTGCCCAACCGTCCAGGGGTCTACAC
CAGTGTGGCCACATATAGCCCCTGGATTCAGGCTCGCGTCAGCTTCTAATGCTAGCCGGTGAGGCTGA
CCTGGAGCCAGCTGCTGGGGTCCCTCAGCCTCCTGGTTCATCCAGGCACCTGCCTATACCCCACATCC

NOV52g, CG56142-02

| | | | |
|---|---|---|---|
| Protein Sequence | SEQ ID NO: 802 | 280 aa | MW at 29786.2kD |

MRGVSCLQVLLLLVLGAAGTQGRKSAACGQPRMSSRIVGGRDGRDGEWPWQASIQHRGAHVCGGSLIA
PQWVLTAAHCFPRRALPAEYRVRLGALRLGSTSPRTLSVPVRRVLLPPDYSEDGARGDLALLQLRRPV
PLSARVQPVCLPVPGARPPPGTPCRVTGWGSLRPGVPLPEWRPLQGVRVPLLDSRTCDGLYHVGADVP
QAERIVLPGSLCAGYPQGHKDACQGDSGGPLTCLQSGSWVLVGVVSWGKGCALPNRPGVYTSVATYSP
WIQARVSF

| | | |
|---|---|---|
| NOV52h, CG56142-03 | SEQ ID NO: 803 | 1606 bp |

| | | |
|---|---|---|
| DNA Sequence | ORF Start: ATG at 69 | ORF Stop: TGA at 921 |

CCACGCGTCCGACCAGAGTCCAAGCCCTAGGCAGTGCCACCCTTACCCAGCCCAGCCTTGAAGACAGA
ATGAGAGGGGTTTCCTGTCTCCAGGTCCTGCTCCTTCTGGTGCTGGGAGCTGCTGGGACTCAGGGAAG
GAAGTCTGCAGCCTGCGGGCAGCCCCGCATGTCCAGTCGGATCGTTGGGGGCCGGGATGGCCGGGACG
GAGAGTGGCCGTGGCAGGCGAGCATCCAGCATCCTGGGGCACACGTGTGCGGGGGGTCGCTCATCGCC
CCCCAGTGGGTGCTGACAGCGGCGCACTGCTTCCCCAGGAGGGCACTGCCAGCTGAGTACCGCGTGCG
CCTGGGGGCGCTGCGTCTGGGCTCCACCTCGCCCCGCACGCTCTCGGTGCCCGTGCGACGGGTGCTGC
TGCCCCCGGACTACTCCGAGGACGGGGCCCGCGGCGACCTGGCACTGCTGCAGCTGCGTCGCCCGGTG
CCCCTGAGCGCTCGCGTCCAACCCGTCTGCCTGCCCGTGCCCGGCGCCCGCCCGCCGCCCGGCACACC
ATGCCGGGTCACCGGCTGGGGCAGCCTCCGCCCAGGAGTGCCCCTCCCAGAGTGGCGACCGCTACAAG
GAGTAAGGGTGCCGCTGCTGGACTCGCGCACCTGCGACGGCCTCTACCACGTGGGCGCGGACGTGCCC
CAGGCTGAGCGCATTGTGCTGCCTGGGAGTCTGTGTGCCGGCTACCCCCAGGGCCACAAGGACGCCTG
CCAGGGTGATTCTGGGGGACCTCTGACCTGCCTGCAGTCTGGGAGCTGGGTCCTGGTGGGCGTGGTGA
GCTGGGGCAAGGGTTGTGCCCTGCCCAACCGTCCAGGGGTCTACACCAGTGTGGCCACATATAGCCCC
TGGATTCAGGCTCGCGTCACTTCTAATGCTAGCCGGTGAGGCTGACCTGGAGCCAGCTGCTGGGGTCC
CTCAGCCTCCTGGTTCATCCAGGCACCTGCCTATACCCCACATCCCTTCTGCCTCGAGGCCAAGATGC
CTAAAAAAGCTAAAGGCCACCCCACCCCCCACCCACCACCTTCTGGCTCCTCTCCTCTTTGGGGATCA
CCAGCTCTGACTCCACCAACCCTCATCCAGGAATCTGCCATGAGTCCCAGGGAGTCACACTCCCCACT
CCCTTCCTGGCTTGTATTTACTTTTCTTGGCCCTGGCCAGGGCTGGGCGCAAGGCACGCAGTGATGGG
CAAACGAATTGCTGCCCATCTGGCCTGTGTGCCCATCTTTTTCTGGAGAAAGTCAGATTCACAGCATG
ACAGAGATTTGACACCAGGGAGATCCTCCATAGCTGGCTTTGAGGACACGGGGACCACAGCCATGAGC
GGCCTCTAAGAGCTGAGAGACAGCCGGCAGGGAATCGGAACCCTCAGACCCACAGCCGCAAGGCACTG
GATTCTGGCAGCACCCTGAAGGAGCTGGGAAGTAAGTTCTTCCCCAGCCTCCAGATAAGAGCCCCGCC
GGCCAATCCCTTCATTTCAACCTAAAGAGACCCTAAGCAGAGAACCTAGCTGAGCCACTCCTGACCTA
CAAAGTTGTGACTTAATAAATGTGTGCTTTAAGCTGCCAAAA

NOV52h, CG56142-03

| | | | |
|---|---|---|---|
| Protein Sequence | SEQ ID NO: 804 | 284 aa | MW at 30109.5kD |

MRGVSCLQVLLLLVLGAAGTQGRKSAACGQPRMSSRIVGGRDGRDGEWPWQASIQHPGAHVCGGSLIA
PQWVLTAAHCFPRRALPAEYRVRLGALRLGSTSPRTLSVPVRRVLLPPDYSEDGARGDLALLQLRRPV
PLSARVQPVCLPVPGARPPPGTPCRVTGWGSLRPGVPLPEWRPLQGVRVPLLDSRTCDGLYHVGADVP
QAERIVLPGSLCAGYPQGHKDACQGDSGGPLTCLQSGSWVLVGVVSWGKGCALPNRPGVYTSVATYSP
WIQARVTSNASR

| | | |
|---|---|---|
| NOV52i, CG56142-05 | SEQ ID NO: 805 | 762 bp |

| | | |
|---|---|---|
| DNA Sequence | ORF Start: at 7 | ORF Stop: at 757 |

AGATCTCAGCCCCGCATGTCCAGTCGGATCGTTGGGGGCCGGGATGGCCGGGACGGAGAGTGGCCGTG
GCAGGCGAGCATCCAGCATCGTGGGGCACACGTGTGCGGGGGGTCGCTCATCGCCCCCCAGTGGGTGC
TGACAGCGGCGCACTGCTTCCCCAGGAGGGCACTGCCAGCTGAGTACCGCGTGCGCCTGGGGGCGCTG
CGTCTGGGCTCCACCTCGCCCCGCACGCTCTCGGTGCCCGTGCGACGGGTGCTGCTGCCCCCGGACTA
CTCCGAGGACGGGGCCCGCGGCGACCTGGCACTGCTGCAGCTGCGTCGCCCGGTGCCCCTGAGCGCTC
GCGTCCAACCCGTCTGCCTGCCCGTGCCCGCCGCCCGCCCGCCGCCCGGCACACCATGCCGGGTCACC
GGCTGGGGCAGCCTCCGCCCAGGAGTGCCCCTCCCAGAGGGGCGACCGCTACAAGGAGTAAGGGTGCC
GCTGCTGGACTCGCGCACCTGCGACGGCCTCTACCACGTGGGCGCGGACGTGCCCCAGGCTGAGCGCA
TTGTGCTGCCTGGGAGTCTGTGTGCCGGCTACCCCCAGGTCCACAAGGACGCCTGCCAGGTGTGCACC
CAGCCTCCCCAGCCTCCGGAGTCCCCTCCCTGTGCCCAGCTCCCTCCCTCCCTGAACTCCAGGACCCA
GGACATCCCAACTCAGGCTCAGGATCCTGGCCTCCAACCTAGAGGCACCACGCCAGGGGTCTGGAACC
CTGAGAACCTCGAG

NOV52i, CG56142-05

| | | | |
|---|---|---|---|
| Protein Sequence | SEQ ID NO: 806 | 250 aa | MW at 26888.5kD |

QPRMSSRIVGGRDGRDGEWPWQASIQHRGAHVCGGSLIAPQWVLTAAHCFPRRALPAEYRVRLGALRL
GSTSPRTLSVPVRRVLLPPDYSEDGARGDLALLQLRRPVPLSARVQPVCLPVPAARPPPGTPCRVTGW
GSLRPGVPLPEGRPLQGVRVPLLDSRTCDGLYHVGADVPQAERIVLPGSLCAGYPQVHKDACQVCTQP
PQPPESPPCAQLPPSLNSRTQDIPTQAQDPGLQPRGTTPGVWNPEN

| | | |
|---|---|---|
| NOV52j, CG56142-06 | SEQ ID NO: 807 | 762 bp |

| | | |
|---|---|---|
| DNA Sequence | ORF Start: at 7 | ORF Stop: at 757 |

AGATCTCAGCCCCGCATGTCCAGTCGGATCGTTGGGGGCCGGGATGGCCGGGACGGAGAGTGGCCGTG
GCAGGCGAGCATCCAGCATCGTGGGGCACACGTGTGCGGGGGGTCGCTCATCGCCCCCCAGTGGGTGC
TGACAGCGGCGCACTGCTTCCCCAGGAGGGCACTGCCAGCTGAGTACCGCGTGCGCCTGGGGGCGCTG
CGTCTGGGCTCCACCTCGCCCCGCACGCTCTCGGTGCCCGTGCGACGGGTGCTGCTGCCCCCGGACTA
CTCCGAGGACGGGGCCCGCGGCGACCTGGCACTGCTGCAGCTGCGTCGCCCGGTGCCCCTGAGCGCTC
GCGTCCAACCCGTCTGCCTGCCCGTGCCCGGCGCCCGCCCGCCGCCCGGCACACCATGCCGGGTCACC
GGCTGGGGCAGCCTCCGCCCAGGAGTGCCCCTCCCAGAGTGGCGACCGCTACAAGGAGTAAGGGTGCC
GCTGCTGGACTCGCGCACCTGCGACGGCCTCTACCACGTGGGCGCGGACGTGCCCCAGGCTGAGCGCA
TTGTGCTGCCTGGGAGTCTGTGTGCCGGCTACCCCCAGGGCCACAAGGACGCCTGCCAGGTGTGCACC
CAGCCTCCCCAGCCTCCGGAGTCCCCTCCCTGTGCCCAGCTCCCTCCCTCCCTGAACTCCAGGACCCA
GGACATCCCAACTCAGGCTCAGGATCCTGGCCTCCAACCTAGAGGCACCACGCCAGGGGTCTGGAACC
CTGAGAACCTCGAG

NOV52j, CG56142-06

| | | | |
|---|---|---|---|
| Protein Sequence | SEQ ID NO: 808 | 250 aa | MW at 26961.6kD |

QPRMSSRIVGGRDGRDGEWPWQASIQHRGAHVCGGSLIAPQWVLTAAHCFPRRALPAEYRVRLGALRL
GSTSPRTLSVPVRRVLLPPDYSEDGARGDLALLQLRRPVPLSARVQPVCLPVPGARPPPGTPCRVTGW
GSLRPGVPLPEWRPLQGVRVPLLDSRTCDGLYHVGADVPQAERIVLPGSLCAGYPQGHKDACQVCTQP
PQPPESPPCAQLPPSLNSRTQDIPTQAQDPGLQPRGTTPGVWNPEN

A ClustalW comparison of the above protein sequences yields the following sequence alignment shown in Table 52B.

TABLE 52B
Comparison of the NOV52 protein sequences.

| | |
|---|---|
| NOV52a | MRGVSCLQVLLLLVL-----------ACGQPRMSSRIVGGRDGRDGEWPWQASIQHRGAH |
| NOV52b | MRGVSCLQVLLLLVLGAAGTQGRKSAACGQPRMSSRIVGGRDGRDGEWPWQASIQHRGAH |
| NOV52c | ------------------------------------------------------------ |
| NOV52d | ------------------------------------------------------------ |
| NOV52e | ------------------------------------------------------------ |
| NOV52f | ------------------------------------------------------------ |
| NOV52g | MRGVSCLQVLLLLVLGAAGTQGRKSAACGQPRMSSRIVGGRDGRDGEWPWQASIQHRGAH |
| NOV52h | MRGVSCLQVLLLLVLGAAGTQGRKSAACGQPRMSSRIVGGRDGRDGEWPWQASIQHRGAH |
| NOV52i | -----------------------------QPRMSSRIVGGRDGRDGEWPWQASIQHRGAH |
| NOV52j | -----------------------------QPRMSSRIVGGRDGRDGEWPWQASIQHRGAH |
| NOV52a | VCGGSLIAPQWVLTAAHCFPR-ALPAEYRVRLGALRLGSTSPRTLSVPVRRVLLPPDYSE |
| NOV52b | VCGGSLIAPQWVLTAAHCFPS--------------------------------------- |
| NOV52c | ------------------------------------------------------------ |
| NOV52d | ------------------------------------------------------------ |
| NOV52e | ------------------------------------------------------------ |
| NOV52f | ------------------------------------------------------------ |
| NOV52g | VCGGSLIAPQWVLTAAHCFPRRALPAEYRVRLGALRLGSTSPRTLSVPVRRVLLPPDYSE |
| NOV52h | VCGGSLIAPQWVLTAAHCFPRRALPAEYRVRLGALRLGSTSPRTLSVPVRRVLLPPDYSE |
| NOV52i | VCGGSLIAPQWVLTAAHCFPRRALPAEYRVRLGALRLGSTSPRTLSVPVRRVLLPPDYSE |
| NOV52j | VCGGSLIAPQWVLTAAHCFPRRALPAEYRVRLGALRLGSTSPRTLSVPVRRVLLPPDYSE |
| NOV52a | DGARGDLALLQLRRPVPLSARVQPVCLPVPGARPPPGTPCRVTGWGSLRPGVPLPEWRPL |
| NOV52b | ---------------------------------------AP-------------PEWRPL |
| NOV52c | --------------------------SAARSTRPPTST-APSSSWA--------LSWRRL |
| NOV52d | --------------------------SAARSTRPPTST-APSSSWA--------LSWRRL |
| NOV52e | --------------------------SAARSTRPPTST-APSSSWA--------LSWRRL |
| NOV52f | --------------------------SAARSTRPPTST-APSSSWA--------LSWRRL |
| NOV52g | DGARGDLALLQLRRPVPLSARVQPVCLPVPGARPPPGTPCRVTGWGSLRPGVPLPEWRPL |
| NOV52h | DGARGDLALLQLRRPVPLSARVQPVCLPVPGARPPPGTPCRVTGWGSLRPGVPLPEWRPL |
| NOV52i | DGARGDLALLQLRRPVPLSARVQPVCLPVPGARPPPGTPCRVTGWGSLRPGVPLPEWRPL |
| NOV52j | DGARGDLALLQLRRPVPLSARVQPVCLPVPGARPPPGTPCRVTGWGSLRPGVPLPEWRPL |
| NOV52a | QGVRVPLLDSRTCDGLYHVG-ADVPQAERIVLPGSLCAGYPQGHKDACQVCTQ-PPQPPE |
| NOV52b | QGVRVPLLDSRTCDGLYHVG-ADVPQAERIVLPGSLCAGYPQGHKDACQVCTQ-PPQPPE |
| NOV52c | GRASQSSHTRVVSSGSLSTGTVTWTCLHRSATPSTPSGGLTPSTCLPRQATTSGLPNTTR |
| NOV52d | GRASQSSHTRVVSSGSLSTGTVTWTCLHRSATPSTPSGGLTPSTCLPRQATTSGLPNTTR |
| NOV52e | GRASQSSHTRVVSSGSLSTGTVTWTCLHRSATPSTPSGGLTPSTCLPRQATTSGLPNTTR |
| NOV52f | GRASQSSHTRVVSSGSLSTGTVTWTCLHRSATPSTPSGGLTPSTCLPRQATTSGLPNTTR |
| NOV52g | QGVRVPLLDSRTCDGLYHVG-ADVPQAERIVLPGSLCAGYPQGHKDACQGDSGGPLTCLQ |
| NOV52h | QGVRVPLLDSRTCDGLYHVG-ADVPQAERIVLPGSLCAGYPQGHKDACQGDSGGPLTCLQ |
| NOV52i | QGVRVPLLDSRTCDGLYHVG-ADVPQAERIVLPGSLCAGYPQVHKDACQVCTQ-PPQPPE |
| NOV52j | QGVRVPLLDSRTCDGLYHVG-ADVPQAERIVLPGSLCAGYPQGHKDACQVCTQ-PPQPPE |
| NOV52a | SPPCAQHPPSLNSRTQDIPTQAQDPGLQPRGTTPGVWNPEN------- |
| NOV52b | SPPCAQHPPSLNSRTQDIPTQAQDPGLQPRGTTPGVWNPEN------- |
| NOV52c | SM--APP-PARSSRPTGSTLTSL-CMDRSGSSA--------------- |
| NOV52d | SM--APP-PARSSRPTGSTLTSL-CMDRSGSSA--------------- |
| NOV52e | SM--APP-PARSSRPTGSTLTSL-CMDRSGSSA--------------- |
| NOV52f | SM--APP-PARSSRPTGSTLTSL-CMDRSGSSA--------------- |
| NOV52g | SG--SWVLVGVVSWGKGCALPNR-PGVYTSVATYSPWIQARVSF---- |
| NOV52h | SG--SWVLVGVVSWGKGCALPNR-PGVYTSVATYSPWIQARVTSNASR |
| NOV52i | SPPCAQLPPSLNSRTQDIPTQAQDPGLQPRGTTPGVWNPEN------- |
| NOV52j | SPPCAQLPPSLNSRTQDIPTQAQDPGLQPRGTTPGVWNPEN------- |
| NOV52a | (SEQ ID NO: 790) |
| NOV52b | (SEQ ID NO: 792) |
| NOV52c | (SEQ ID NO: 794) |
| NOV52d | (SEQ ID NO: 796) |
| NOV52e | (SEQ ID NO: 798) |
| NOV52f | (SEQ ID NO: 800) |
| NOV52g | (SEQ ID NO: 802) |
| NOV52h | (SEQ ID NO: 804) |
| NOV52i | (SEQ ID NO: 806) |
| NOV52j | (SEQ ID NO: 808) |

Further analysis of the NOV52a protein yielded the following properties shown in Table 52C.

TABLE 52C
Protein Sequence Properties NOV52a

| | |
|---|---|
| SignalP analysis: | Cleavage site between residues 19 and 20 |
| PSORT II analysis: | |

| | |
|---|---|
| PSG: | a new signal peptide prediction method |
| | N-region: length 2; pos.chg 1; neg.chg 0 |
| | H-region: length 18; peak value 10.88 |
| | PSG score: 6.48 |
| GvH: | von Heijne's method for signal seq. recognition |
| | GvH score (threshold: −2.1): 1.06 |
| | possible cleavage site: between 18 and 19 |

>>> Seems to have a cleavable signal peptide (1 to 18)

| | |
|---|---|
| ALOM: | Klein et al's method for TM region allocation |
| | Init position for calculation: 19 |
| | Tentative number of TMS(s) for the threshold 0.5: 0 |
| | number of TMS(s) .. fixed |
| | PERIPHERAL Likelihood = 5.30 (at 48) |
| | ALOM score: 5.30 (number of TMSs: 0) |
| MTOP: | Prediction of membrane topology (Hartmann et al.) |
| | Center position for calculation: 9 |
| | Charge difference: 0.0 C( 2.0) - N( 2.0) |
| | N >= C: N-terminal side will be inside |

MITDISC: discrimination of mitochondrial targeting seq

| | | | |
|---|---|---|---|
| R content: | 4 | Hyd Moment (75): | 6.32 |
| Hyd Moment (95): | 5.75 | G content: | 4 |
| D/E content: | 1 | S/T content: | 3 |
| Score: −2.36 | | | |

Gavel: prediction of cleavage sites for mitochondrial preseq

R-2 motif at 35 SRI|VG

NUCDISC: discrimination of nuclear localization signals

pat4: none
pat7: none
bipartite: none
content of basic residues: 10.1%
NLS Score: −0.47

KDEL: ER retention motif in the C-terminus: none
ER Membrane Retention Signals:

XXRR-like motif in the N-terminus: RGVS

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 76.7

COIL: Lupas's algorithm to detect coiled-coil regions

total: 0 residues

--------------------------
Final Results (k = 9/23):

55.6%: extracellular, including cell wall
33.3%: mitochondrial
11.1%: nuclear

>> prediction for CG56142-01 is exc (k = 9)

A search of the NOV52a protein against the Geneseq database, a proprietary database that contains sequences published in patents and patent publication, yielded several homologous proteins shown in Table 52D.

TABLE 50D
Geneseq Results for NOV52a

| | | NOV50a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU74742 | Human protease PRTS-2 protein | 1..267 | 267/279 (95%) | e-159 |
| | sequence - Homo sapiens, 365 aa. | 87..365 | 267/279 (95%) | |
| | [WO200 1 98468-A2, 27-Dec.- | | | |
| | 2001] | | | |
| ABP61012 | Novel human protein. | 1..267 | 267/279 (95%) | e-159 |
| | SEQ ID 99 - | 1..279 | 267/279 (95%) | |
| | Homo sapiens, 279 aa. | | | |
| | [WO200250 105-Al, 27-Jun.- | | | |
| | 2002] | | | |
| AAE00290 | Human plasminogen-like protein | 1..267 | 267/279 (95%) | e- 159 |
| | from clone HEOQBO8 - Homo | 1..279 | 267/279 (95%) | |
| | sapiens, 279 aa. [WO200124815- | | | |
| | Al, 12-Apr.-2001] | | | |
| ABB07286 | Human prostasin-like serine | 1..216 | 216/217 (99%) | e-127 |
| | protease - Homo sapiens, 272 aa. | 1..217 | 216/217 (99%) | |
| | [WO2001 98466-A2, 27-Dec.- | | | |
| | 2001] | | | |
| ABP61010 | Novel human protein. | 1..216 | 216/228 (94%) | e-125 |
| | SEQ ID 97 - | 1..228 | 216/228 (94%) | |
| | Homo sapiens, 280 aa. | | | |
| | [WO200250105-A1, 27-Jun.- | | | |
| | 2002] | | | |

In a BLAST search of public sequence databases, the NOV52a protein was found to have homology to the proteins shown in the BLASTP data in Table 52E.

TABLE 52E
Public BLASTP Results for NOV52a

| | | NOV52a | Identities/ | |
|---|---|---|---|---|
| Protein | | Residues/ | Similarities for | Ex- |
| Accession | Protein/ | Match | the Matched | pect |
| Number | Organism/Length | Residues | Portion | Value |
| Q8N171 | Similar to protease, | 1..216 | 216/228 (94%) | e-124 |
| | serine, 8 | 48..275 | 216/228 (94%) | |
| | (Prostasin) - | | | |
| | Homo sapiens | | | |
| | (Human), 327 aa | | | |
| | (fragment). | | | |
| Q8NF86 | Serine protease | 1..216 | 215/228 (94%) | e-123 |
| | EOS - Homo sapiens | 1..228 | 215/228 (94%) | |
| | (Human), 284 aa. | | | |
| AAP20885 | Tryptase-6 - | 1..216 | 175/225 (77%) | 3e-98 |
| | Mus musculus | 1..225 | 186/225 (81%) | |
| | (Mouse), 277 aa. | | | |
| Q9ER10 | Brain-specific serine | 17..215 | 91/202 (45%) | 1e-46 |
| | protease 4 precursor | 41..238 | 125/202 (61%) | |
| | (EG 3.4.21.-) | | | |
| | (BSSP-4) - | | | |
| | Mus musculus | | | |
| | (Mouse), 306 aa. | | | |
| Q9ES87 | Prostasin precursor | 16..216 | 93/202 (46%) | 1e-46 |
| | (EC 3.4.21.-) - | 36..235 | 125/202 (61%) | |
| | Rattus norvegicus | | | |
| | (Rat), 342 aa. | | | |

PFam analysis indicates that the NOV52a protein contains the domains shown in the Table 52F.

TABLE 52F
Domain Analysis of NOV52a

| | | Identities/ | |
|---|---|---|---|
| | NOV52a | Similarities | Expect |
| Pfam Domain | Match Region | for the Matched Region | Value |
| trypsin | 26 . . . 241 | 83/269 (31%) | 1e−38 |
| | | 165/269 (61%) | |

### Example 53

The NOV53 clone was analyzed, and the nucleotide and encoded polypeptide sequences are shown in Table 53A.

TABLE 53A
NOV53 Sequence Analysis

| | | |
|---|---|---|
| NOV53a, CG56144-01 | SEQ ID NO: 809 | 1151 bp |

| | | |
|---|---|---|
| DNA Sequence | ORF Start: ATG at 90 | ORF Stop: TAG at 1056 |

TAATCTTTGCAGGTGGGATAGCACAGGTTGAACTCTAATCATATATACTGTAGAAGGTATATATAGAA
GGTGAAGAAGCCCTGTAAAAAATGACAAGGAGATTTCCAGGAGCCATGCTTCCCTCTAATATCACCTC
AACACATCCAGCTGTCTTTTTGTTGGTAGGAATTCCTGGTTTGGAACACCTGCATGCCTGGATCTCCA
TCCCCTTCTGCTTTGCTTATACTCTGGCCCTGCTAGGCAACTGTACCCTTCTCTTCATTATCCAGGCT
GATGCAGCCCTCCATGAACCCATGTACCTCTTTCTGGCCATGTTGGCAACCATTGACTTGGTTCTTTC
TTCTACAACGCTGCCCAAAATGCTTGCCATATTCTGGTTCAGGGATCAGGAGATCAACTTCTTTGCCT
GTCTGGTCCAGATGTTCTTCCTTCACTCCTTCTCCATCATGGAGTCAGCAGTGCTGCTGGCCATGGCC
TTTGACCGCTATGTGGCCATCTGCAAGCCATTGCACTACACGACGGTCCTGACTGGGTCCCTCATCAC
CAAGATTGGCATGGCTGCTGTGGCCCGGGCTGTGACACTAATGACTCCACTCCCCTTCCTGCTCAGAC
GCTTCCACTACTGCCGAGGCCCAGTGATTGCCCATTGCTACTGTGAACACATGGCTGTGGTAAGGCTG
GCGTGTGGGGACACTAGCTTCAACAATATCTATGGCATTGCTGTGGCCATGTTTAGTGTGGTGTTGGA
CCTGCTCTTTGTTATCCTGTCTTATGTCTTCATCCTTCAGGCAGTTCTCCAGCTTGCCTCTCAGGAGG
CCCGCTACAAAGCATTTGGGACATGTGTGTCTCACATAGGTGCCATCCTGTCCACCTACACTCCAGTA
GTCATCTCTTCAGTCATGCACCGTGTAGCCCGCCATGCTGCCCCTCGTGTCCACATACTCCTTGCTAT
TTTCTATCTCCTTTTCCCACCCATGGTCAATCCTATCATATATGGAGTCAAGACCAAGCAGATTCGTG
AGTATGTGCTCAGTCTATTCCAGAGAAAGAACATGTAGATGGATAGTTCTCTTTTTTTATCCCACTTG
CCAAGTAATGAGAATGCTGGATTGGGGTTGAGGGGAAAAATCTAAATAGGAAAATTGCAGAGT

NOV53a, CG56144-01

| | | | |
|---|---|---|---|
| Protein Sequence | SEQ ID NO: 810 | 322 aa | MW at 36121.9kD |

MTRRFPGAMLPSNITSTHPAVFLLVGIPGLEHLHAWISIPFCFAYTLALLGNCTLLFIIQADAALHEP
MYLFLAMLATIDLVLSSTTLPKMLAIFWFRDQEINFFACLVQHFFLHSFSIMESAVLLAMAFDRYVAI
CKPLHYTTVLTGSLITKIGMAAVARAVTLMTPLPFLLRRFHYCRGPVIAHCYCEHMAVVRLACGDTSF
NNIYGIAVAMFSVVLDLLFVILSYVFILQAVLQLASQEARYKAFGTCVSHIGAILSTYTPVVISSVMH
RVARHAAPRVHILLAIFYLLFPPMVNPIIYGVKTKQIREYVLSLFQRKNM

| | | |
|---|---|---|
| NOV53b, 170645965 | SEQ ID NO: 811 | 981 bp |

| | | |
|---|---|---|
| DNA Sequence | ORF Start: at 1 | ORF Stop: end of sequence |

GGATCCACCATGACAAGGAGATTTCCAGGAGCCATGCTTCCCTCTAATATCACCTCAACACATCCAGC
TGTCTTTTTGTTGGTAGGAATTCCTGGTTTGGAACACCTGCATGCCTGGATCTCCATCCCCTTCTGCT
TTGCTTATACTCTGGCCCTGCTAGGCAACTGTACCCTTCTCTTCATTATCCAGGCTGATGCAGCCCTC
CATGAACCCATGTACCTCTTTCTGGCCATGTTGGCAACCATTGACTTGGTTCTTTCTTCTACAACGCT
GCCCAAAATGCTTGCCATATTCTGGTTCAGGGATCAGGAGATCAACTTCTTTGCCTGTCTGGTCCAGA
TGTTCTTCCTTCACTCCTTCTCCATCATGGAGTCAGCAGTGCTGCTGGCCATGGCCTTTGACCGCTAT
GTGGCCATCTGCAAGCCATTGCACTACACGACGGTCCTGACTGGGTCCCTCATCACCAAGATTGGCAT
GGCTGCTGTGGCCCGGGCTGTGACACTAATGACTCCACTCCCCTTCCTGCTCAGACGCTTCCACTACT
GCCGAGGCCCAGTGATTGCCCATTGCTACTGTGAACACATGGCTGTGGTAAGGCTGGCGTGTGGGGAC
ACTAGCTTCAACAATATCTATGGCATTGCTGTGGCCATGTTTATTGTGGTGTTGGACCTGCTCTTTGT
TATCCTGTCTTATGTCTTCATCCTTCAGGCAGTTCTCCAGCTTGCCTCTCAGGAGGCCCGCTACAAGG
CATTTGGGACATGTGTGTCTCACATAGGTGCCATCCTGTCCACCTACACTCCAGTAGTCATCTCTTCA
GTCATGCACCGTGTAGCCCGCCATGCTGCCCCTCGTGTCCACATACTCCTTGCTATTTTCTATCTCCT
TTTCCCACCCATGGTCAATCCTATCATATATGGAGTCAAGACCAAGCAGATTCGTGAGTATGTGCTCA
GTCTATTCCAGAGAAAGAACATGCTCGAG

NOV53b, 170645965

| | | | |
|---|---|---|---|
| Protein Sequence | SEQ ID NO: 812 | 327 aa | MW at 36635.5kD |

GSTMTRRFPGAMLPSNITSTHPAVFLLVGIPGLEHLHAWISIPFCFAYTLALLGNCTLLFIIQADAAL
HEPMYLFLAMLATIDLVLSSTTLPKMLAIFWFRDQEINFFACLVQMFFLHSFSIMESAVLLAMAFDRY
VAICKPLHYTTVLTGSLITKIGMAAVARAVTLMTPLPFLLRRFHYCRGPVIAHCYCEHMAVVRLACGD
TSFNNIYGIAVAMFIVVLDLLFVILSYVFILQAVLQLASQEARYKAFGTCVSHIGAILSTYTPVVISS
VMHRVARHAAPRVHILLAIFYLLFPPMVNPIIYGVKTKQIREYVLSLFQRKNMLE

| | | |
|---|---|---|
| NOV53c, 168869277 | SEQ ID NO: 813 | 981 bp |

| | | |
|---|---|---|
| DNA Sequence | ORF Start: at 1 | ORF Stop: end of sequence |

GGATCCACCATGACAAGGAGATTTCCAGGAGCCATGCTTCCCTCTAATATCACCTCAACACATCCAGC
TGTCTTTTTGTTGGTAGGAATTCCTGGTTTGGAACACCTGCATGCCTGGATCTCCATCCCCTTCTGCT
TTGCTTATACTCTGGCCCTGCTAGGCAACTGTACCCTTCTCTTCATTATCCAGGCTGATGCAGCCCTC
CATGAACCCATGTACCTCTTTCTGGCCATGTTGGCAACCATTGACTTGGTTCTTTCTTCTACAACGCT
GCCCAAAATGCTTGCCATATTCTGGTTCAGGGATCAGGAGATCAACTTCTTTGCCTGTCTGGTCCAGA
TGTTCTTCCTTCACTCCTTCTCCATCATGGAGTCAGCAGTGCTGCTGGCCATGGCCTTTGACCGCTAT
GTGGCCATCTGCAAGCCATTGCACTACACGACGGTCCTGACTGGGTCCCTCATCACCAAGATTGGCAT
GGCTGCTGTGGCCCGGGCTGTGACACTAATGACTCCACTCCCCTTCCTGCTCAGACGCTTCCACTACT
GCCGAGGCCCAGTGATTGCCCATTGCTACTGTGAACACATGGCTGTGGTAAGGCTGGCGTGTGGGGAC
ACTAGCTTCAACAATATCTATGGCATTGCTGTGGCCATGTTTATTGTGGTGTTGGACCTGCTCTTTGT
TATCCTGTCTTATGTCTTCATCCTTCAGGCAGTTCTCCAGCTTGCCTCTCAGGAGGCCCGCTACAAGG
CATTTGGGACATGTGCGTCTCACATAGGTGCCATCCTGTCCACCTACACTCCAGTAGTCATCTCTTCA
GTCATGCACCGTGTAGCCCGCCATGCTGCCCCTCGTGTCCACATACTCCTTGCTATTTTCTATCTCCT
TTTCCCACCCATGGTCAATCCTATCATATATGGAGTCAAGACCAAGCAGATTCGTGAGTATGTGCTCA
GTCTATTCCAGAGAAAGAACATGCTCGAG

NOV53c, 168869277

| | | | |
|---|---|---|---|
| Protein Sequence | SEQ ID NO: 814 | 327 aa | MW at 36607.4kD |

GSTMTRRFPGAMLPSNITSTHPAVFLLVGIPGLEHLHAWISIPFCFAYTLALLGNCTLLFIIQADAAL
HEPMYLFLAMLATIDLVLSSTTLPKMLAIFWFRDQEINFFACLVQMFFLHSFSIMESAVLLAMAFDRY
VAICKPLHYTTVLTGSLITKIGMAAVARAVTLMTPLPFLLRRFHYCRGPVIAHCYCEHMAVVRLACGD
TSFNNIYGIAVAMFIVVLDLLFVILSYVFILQAVLQLASQEARYKAFGTCASHIGAILSTYTPVVISS
VMHRVARHAAPRVHILLAIFYLLFPPMVNPIIYGVKTKQIREYVLSLFQRKNNLE

| | | |
|---|---|---|
| NOV53d, 170645981 | SEQ ID NO: 815 | 981 bp |

| | | |
|---|---|---|
| DNA Sequence | ORF Start: at 1 | ORF Stop: end of sequence |

GGATCCACCAAGACAAGGAGATTTCCAGGAGCCATGCTTCCCTCTAATATCACCTCAACACATCCAGC
TGTCTTTTTGTTGGTAGGAATTCCTGGTTTGGAACACCTGGATGCCTGGATCTCCATCCCCTTCTGCT
TTGCTTATACTCTGGCCCTGCTAGGCAACTGTACCCTTCTCTTCATTATCCAGGCTGATGCAGCCCTC
CATGAACCCATGTACCTCTTTCTGGCCATGTTGGCAACCATTGACTTGGTTCTTTCTTCTACAACGCT
GCCCAAAATGCTTGCCATATTCTGGTTCAGGGATCAGGAGATCAACTTCTTTGCCTGTCTGGTCCAGA
TGTTCTTCCTTCACTCCTTCTCCATCATGGAGTCAGCAGTGCTGCTGGCCATGGCCTTTGACCGCTAT
GTGGCCATCTGCAAGCCATTGCACTACACGACGGTCCTGACTGGGTCCCTCATCACCAAGATTGGCAT
GGCTGCTGTGGCCCGGGCTGTGACACTAATGACTCCACTCCCCTTCCTGCTCAGACGCTTCCACTACT
GCCGAGACCCAGTGATTGCCCATTGCTACTGTGAACACATGGCTGTGGTAAGGCTGGCGTGTGGGGAC
ACTAGCTTCAACAATATCTATGGCATTGCTGTGGCCATGTTTATTGTGGTGTTGGACCTGCTCTTTGT
TATCCTGTCTTATGTCTTCATCCTTCAGGCAGTTCTCCAGCTTGCCTCTCAGGAGGCCCGCTACAAGG
CATTTGGGACATGTGTGTCTCACATAGGTACCATCCTGTCCACCTACACTCCAGTAGTCATCTCTTCA
GTCATGCACCGTGTAGCCCGCCATGCTGCCCCTCGTGTCCACATACTCCTTGCTATTTTCTATCTCCT
TTTCCCACCCATGGTCAATCCTATCATATATGGAGTCAAGACCAAGCAGATTCGTGAGTATGTGCTCA
GTCTATTCCAGAGAAAGAACATGCTCGAG

NOV53d, 170645981

| | | | |
|---|---|---|---|
| Protein Sequence | SEQ ID NO: 816 | 327 aa | MW at 36720.5kD |

GSTKTRRFPGAMLPSNITSTHPAVFLLVGIPGLEHLHAWISIPFCFAYTLALLGNCTLLFIIQADAAL
HEPMYLFLAMLATIDLVLSSTTLPKMLAIFWFRDQEINFFACLVQMFFLHSFSIMESAVLLAMAFDRY
VAICKPLHYTTVLTGSLITKIGMAAVARAVTLMTPLPFLLRRFHYCRDPVIAHCYCEHMAVVRLACGD
TSFNNIYGIAVAMFIVVLDLLFVILSYVFILQAVLQLASQEARYKAFGTCVSHIGTILSTYTPVVISS
VMHRVARHAAPRVHILLAIFYLLFPPMVNPIIYGVKTKQIREYVLSLFQRKNMLE

| | | |
|---|---|---|
| NOV53e, 168869262 | SEQ ID NO: 817 | 981 bp |

| | | |
|---|---|---|
| DNA Sequence | ORF Start: at 1 | ORF Stop: end of sequence |

GGATCCACCATGACAAGGAGATTTCCAGGAGCCATGCTTCCCTCTAATATCACCTCAACACATCCAGC
TGTCTTTTTGTTGGTAGGAATTCCTGGTTTGGAACACCTGCATGCCTGGATCTCCATCCCCTTCTGCT
TTGCTTATACTCTGGCCCTGCTAGGCAACTGTACCCTTCTCTTCATTATCCGGGCTGATGCAGCCCTC
CATGAACCCATGTACCTCTTTCTGGCCATGTTGGCAACCATTGACTTGGTTCTTTCTTCTACAACGCT
GCCCAAAATGCTTGCCATATTCTGGTTCAGGGATCAGGAGATCAACTTCTTTGCCTGTCTGGTCCAGA
TGTTCTTCCTTCACTCCTTCTCCATCATGGAGTCAGCAGTGCTGCTGGCCATGGCCTTTGACCGCTAT
GTGGCCATCTGCAAGCCATTGCACTACACGACGGTCCTGACTGGGTCCCTCATCACCAAGATTGGCAT
GGCTGCTGTGGCCTGGGCTGTGACACTAATGACTCCACTCCCCTTCCTGCTCAGACGCTTCCACTACT
GCCGAGGCCCAGTGATTGCCCATTGCTACTGTGAACACATGGCTGTGGTAAGGCTGGCGTGTGGGGAC
ACTAGCTTCAACAATATCTATGGCATTGCTGTGGCCATGTTTATTGTGGTGTTGGACCTGCTCTTTGT
TATCCTGTCTTATGTCTTCATCCTTCAGGCAGTTCTCCAGCTTGCCTCTCAGGAGGCCCGCTACAAGG
CATTTGGGACATGTGTGTCTCACATAGGTGCCATCCTGTCCACCTACACTCCAGTAGTCATCTCTTCA
GTCATGCACCGTGTAGCCCGCCATGCTGCCCCTCGTGTCCACATACTCCTTGCTATTTTCTATCTCCT
TTTCCCACCCATGGTCAATCCTATCATATATGGAGTCAAGACCAAGCAGATTCGTGAGTATGTGCTCA
GTCTATTCCAGAGAAAGAACATGCTCGAG

NOV53e, 168869262

| | | | |
|---|---|---|---|
| Protein Sequence | SEQ ID NO: 818 | 327 aa | MW at 36693.5kD |

GSTHTRRFPGAMLPSNITSTHPAVFLLVGIPGLEHLHAWISIPFCFAYTLALLGMCTLLFIIRADAAL
HEPMYLFLAMLATIDLVLSSTTLPKMLAIFWFRDQEINFFACLVQMFFLHSFSIMESAVLLAMAFDRY
VAICKPLHYTTVLTGSLITKIGMAAVAWAVTLMTPLPFLLRRFHYCRGPVIAHCYCEHMAVVRLACGD
TSFNNIYGIAVAMFIVVLDLLFVILSYVFILQAVLQLASQEARYKAFGTCVSHIGAILSTYTPVVISS
VMHRVARHAAPRVHILLAIFYLLFPPMVNPIIYGVKTKQIREYVLSLFQRKNMLE

| | | |
|---|---|---|
| NOV53f, 168869254 | SEQ ID NO:819 | 981 bp |

| | | |
|---|---|---|
| DNA Sequence | ORF Start: at 1 | ORF Stop: end of sequence |

GGATCCACCATGACAAGGAGATTTCCAGGAGCCATGCTTCCCTCTAATATCACCTCAACACATCCAGC
TGTCTTTTTGTTGGTAGGAATTCCTGGTTTGGAACACCTGCATGCCTGGATCTCCATCCCCCTCTGCT
TTGCTTATACTCTGGCCCTGCTAGGCAACTGTACCCTTCTCTTCATTATCCAGGCTGATGCAGCCCTC
CATGAACCCATGTACCTCTTTCTGGCCATGTTGGCAACCATTGACTTGGTTCTTTCTTCTACAACGCT
GCCCAAAATGCTTGCCATATTCTGGTTCAGGGATCAGGAGATCAACTTCTTTGCCTGTCTGGTCCAGA
TGTTCTTCCTTCACTCCTTCTCCATCATGGAGTCAGCAGTGCTGCTGGCCATGGCCTTTGACCGCTAT
GTGGCCATCTGCAAGCCATTGCACTACACGACGGTCCTGACTGGGTCCCTCATCACCAAGATTGGCAT
GGCTGCTGTGGCCCGGGCTGTGACACTAATGACTCCACTCCCCTTCCTGCTCAGACGCTTCCACTACT
GCCGAGGCCCAGTGATTGCCCATTGCTACTGTGAACACATGGCTGTGGTAAGGCTGGCGTGTGGGGAC
ACTAGCTTCAACAATATCTATGGCATTGCTGTGGCCATGTTTATTGTGGTGTTGGACCTGCTCTTTGT
TATCCTGTCTTATGTCTTCATCCTTCAGGCAGTTCTCCAGCTTGCCTCTCAGGAGGCCCGCTACAAGG
CATTTGGGACATGTGTGTCTCACATAGGTGCCATCCTGTCCACCTACACTCCAGTAGTCATCTCTTCA
GTCATGCACCGTGTAGCCCGCCATGCTGCCCCTCGTGTCCACATACTCCTTGCTATTTTCTATCTCCT
TTTCCCACCCATGGTCAATCCTATCATATATGGAGTCAAGACCAAGCAGATTCGTGAGTATGTGCTCA
GTCTATTCCAGAGAAAGAACATGCTCGAG

NOV53f, 168869254

| | | | |
|---|---|---|---|
| Protein Sequence | SEQ ID NO: 820 | 327 aa | MW at 36601.4kD |

GSTMTRRFPGANLPSNITSTHPAVFLLVGIPGLEHLHAWISIPLCFAYTLALLGNCTLLFIIQADAAL
HEPMYLFLAMLATIDLVLSSTTLPKMLAIFWFRDQEINFFACLVQMFFLHSFSIMESAVLLAMAFDRY
VAICKPLHYTTVLTGSLITKIGMAAVARAVTLMTPLPFLLRRFHYCRGPVIAHCYCEHMAVVRLACGD
TSFNNIYGIAVANFIVVLDLLFVILSYVFILQAVLQLASQEARYKAFGTCVSHIGAILSTYTPVVISS
VMHRVARHAAPRVHILLAIFYLLFPPMVNPIIYGVKTKQIREYVLSLFQRKNMLE

| | | |
|---|---|---|
| NOV53g, CG56144-02 | SEQ ID NO: 821 | 966 bp |

| | | |
|---|---|---|
| DNA Sequence | ORF Start: ATG at 1 | ORF Stop: end of sequence |

ATGACAAGGAGATTTCCAGGAGCCATGCTTCCCTCTAATATCACCTCAACACATCCAGCTGTCTTTTT
GTTGGTAGGAATTCCTGGTTTGGAACACCTGCATGCCTGGATCTCCATCCCCTTCTGCTTTGCTTATA
CTCTGGCCCTGCTAGGCAACTGTACCCTTCTCTTCATTATCCAGGCTGATGCAGCCCTCCATGAACCC
ATGTACCTCTTTCTGGCCATGTTGGCAACCATTGACTTGGTTCTTTCTTCTACAACGCTGCCCAAAAT
GCTTGCCATATTCTGGTTCAGGGATCAGGAGATCAACTTCTTTGCCTGTCTGGTCCAGATGTTCTTCC
TTCACTCCTTCTCCATCATGGAGTCAGCAGTGCTGCTGGCCATGGCCTTTGACCGCTATGTGGCCATC
TGCAAGCCATTGCACTACACGACGGTCCTGACTGGGTCCCTCATCACCAAGATTGGCATGGCTGCTGT
GGCCCGGGCTGTGACACTAATGACTCCACTCCCCTTCCTGCTCAGACGCTTCCACTACTGCCGAGGCC
CAGTGATTGCCCATTGCTACTGTGAACACATGGCTGTGGTAAGGCTGGCGTGTGGGGACACTAGCTTC
AACAATATCTATGGCATTGCTGTGGCCATGTTTATTGTGGTGTTGGACCTGCTCTTTGTTATCCTGTC
TTATGTCTTCATCCTTCAGGCAGTTCTCCAGCTTGCCTCTCAGGAGGCCCGCTACAAGGCATTTGGGA
CATGTGTGTCTCACATAGGTGCCATCCTGTCCACCTACACTCCAGTAGTCATCTCTTCAGTCATGCAC
CGTGTAGCCCGCCATGCTGCCCCTCGTGTCCACATACTTCTTGCTATTTTCTATCTCCTTTTCCCACC
CATGGTCAATCCTATCATATATGGAGTCAAGACCAAGCAGATTCGTGAGTATGTGCTCAGTCTATTCC
AGAGAAAGAACATG

NOV53g, CG56144-02

| | | | |
|---|---|---|---|
| Protein Sequence | SEQ ID NO: 822 | 322 aa | MW at 36148.0kD |

MTRRFPGAMLPSNITSTHPAVFLLVGIPGLEHLHAWISIPFCFAYTLALLGNCTLLFIIQADAALHEP
MYLFLAMLATIDLVLSSTTLPKMLAIFWFRDQEINFFACLVQMFFLHSFSIMESAVLLAMAFDRYVAI
CKPLHYTTVLTGSLITKIGMAAVARAVTLMTPLPFLLRRFHYCRGPVIAHCYCEHMAVVRLACGDTSF
NNIYGIAVAMFIVVLDLLFVILSYVFILQAVLQLASQEARYKAFGTCVSHIGAILSTYTPVVISSVMH
RVARHAAPRVHILLAIFYLLFPPMVNPIIYGVKTKQIREYVLSLFQRKNM

| | | |
|---|---|---|
| NOV53h, CG56144-03 | SEQ ID NO: 823 | 777 bp |

| | | |
|---|---|---|
| DNA Sequence | ORF Start: at 1 | ORF Stop: end of sequence |

GCCCTCCATGAACCCATGTACCTCTTTCTGGCCATGTTGGCAACCATTGACTTGGTTCTTTCTTCTAC
AACGCTGCCCAAAATGCTTGCCATATTCTGGTTCAGGGATCAGGAGATCAACTTCTTTGCCTGTCTGG
TCCAGATGTTCTTCCTTCACTCCTTCTCCATCATGGAGTCAGCAGTGCTGCTGGCCATGGCCTTTGAC
CGCTATGTGGCCATCTGCAAGCCATTGCACTACACGACGGTCCTGACTGGGTCCCTCATCACCAAGAT
TGGCATGGCTGCTGTGGCCTGGGCTGTGACACTAATGACTCCACTCCCCTTCCTGCTCAGACGCTTCC
ACTACTGCCGAGGCCCAGTGATTGCCCATTGCTACTGTGAACACATGGCTGTGGTAAGGCTGGCGTGT
GGGGACACTAGCTTCAACAATATCTATGGCATTGCTGTGGCCATGTCTATTGTGGTGTTGGACCTGCT
CTTTGTTATCCTGTCTTATGTCTTCATCCTTCAGGCAGTTCTCCAGCTTGCCTCTCAGGAGGCCCGCT
ACAAGGCATTTGGGACATGTGTGTCTCACATAGGTGCCATCCTGTCCACCTACACTCCAGTAGTCATC
TCTTCAGTCATGCACCGTGTAGCCCGCCATGCTGCCCCTCGTGTCCACATACTCCTTGCTATTTTCTA
TCTCCTTTTCCCACCCATGGTCAATCCTATCATATATGGAGTCAAGACCAAGCAGATTCGTGAGTATG
TGCTCAGTCTATTCCAGAGAAAGAACATG

NOV53h, CG56144-03

| | | | |
|---|---|---|---|
| Protein Sequence | SEQ ID NO: 824 | 259 aa | MW at 29252.8kD |

ALHEPMYLFLAMLATIDLVLSSTTLPKMLAIFWFRDQEINFFACLVQMFFLHSFSIMESAVLLAMAFD
RYVAICKPLHYTTVLTGSLITKIGMAAVAWAVTLMTPLPFLLRRFHYCRGPVIAHCYCEHMAVVRLAC
GDTSFNNIYGIAVAMSIVVLDLLFVILSYVFILQAVLQLASQEARYKAFGTCVSHIGAILSTYTPVVI
SSVMHRVARHAAPRVHILLAIFYLLFPPMVNPIIYGVKTKQIREYVLSLFQRKNM

| | | |
|---|---|---|
| NOV53i, CG56144-04 | SEQ ID NO: 825 | 777 bp |

| | | |
|---|---|---|
| DNA Sequence | ORF Start: at 1 | ORF Stop: end of sequence |

GCCCTCCATGAACCCATGTACCTCTTTCTGGCCATGTTGGCAACCATTGACTTGGTTCTTTCTTCTAC
AACGCTGCCCAAAATGCTTGCCATATTCTGGTTCAGGGATCAGGAGATCAACTTCTTTGCCTGTCTGG
TCCAGATGTTCTTCCTTCACTCCTTCTCCATCATGGAGTCAGCAGTGCTGCTGGCCATGGCCTTTGAC
CGCTATGTGGCCATCTGCAAGCCATTGCACTACACGACGGTCCTGACTGGGTCCCTCATCACCAAGAT
TGGCATGGCTGCTGTGGCCTGGGCTGTGACACTAATGACTCCACTCCCCTTCCTGCTCAGACGCTTCC
ACTACTGCCGAGGCCCAGTGATTGCCCATTGCTACTGTGAACACATGGCTGTGGTAAGGCTGGCGTGT
GGGGACACTAGCTTCAACAATATCTATGGCATTGCTGTGGCCATGTCTATTGTGGTGTTGGACCTGCT
CTTTGTTATCCTGTCTTATGTCTTCATCCTTCAGGCAGTTCTCCAGCTTGCCTCTCAGGAGGCCCGCT
ACAAGGCATTTGGGACATGTGTGTCTCACATAGGTGCCATCCTGTCCACCTACACTCCAGTAGTCATC
TCTTCAGTCATGCACCGTGTAGCCCGCCATGCTGCCCCTCGTGTCCACATACTCCTTGCTATTTTCTA
TCTCCTTTTCCCACCCATGGTCAATCCTATCATATATGGAGTCAAGACCAAGCAGATTCGTGAGTATG
TGCTCAGTCTATTCCAGAGAAAGAACATG

NOV53i, CG56144-04

| | | | |
|---|---|---|---|
| Protein Sequence | SEQ ID NO: 826 | 259 aa | MW at 29252.8kD |

ALHEPMYLFLAMLATIDLVLSSTTLPKMLAIFWFRDQEINFFACLVQMFFLHSFSIMESAVLLAMAFD
RYVAICKPLHYTTVLTGSLITKIGMAAVAWAVTLMTPLPFLLRRFHYCRGPVIAHCYCEHMAVVRLAC
GDTSFNNIYGIAVAMSIVVLDLLFVILSYVFILQAVLQLASQEARYKAFGTCVSHIGAILSTYTPVVI
SSVMHRVARHAAPRVHILLAIFYLLFPPMVNPIIYGVKTKQIREYVLSLFQRKNM

| | | |
|---|---|---|
| NOV53j, CG56144-05 | SEQ ID NO: 827 | 789 bp |

| | | |
|---|---|---|
| DNA Sequence | ORF Start: at 7 | ORF Stop: at 784 |

GGATCCGCCCTCCATGAACCCATGTACCTCTTTCTGGCCATGTTGGCAACCATTGACTTGGTTCTTTC
TTCTACAACGCTGCCCAAAATGCTTGCCATATTCTGGTTCAGGGATCAGGAGATCAACTTCTTTGCCT
GTCTGGTCCAGATGTTCTTCCTTCACTCCTTCTCCATCATGGAGTCAGCAGTGCTGCTGGCCATGGCC
TTTGACCGCTATGTGGCCATCTGCAAGCCATTGCACTACACGACGGTCCTGACTGGGTCCCTCATCAC
CAAGATTGGCATGGCTGCTGTGGCCCGGGCTGTGACACTAATGACTCCACTCCCCTTCCTGCTCAGAC
GCTTCCACTACTGCCGAGGCCCAGTGATTGCCCATTGCTACTGTGAACACATGGCTGTGGTAAGGCTG
GCGTGTGGGGACACTAGCTTCAACAATATCTATGGCATTGCTGTGGCCATGTTTATTGTGGTGTTGGA
CCTGCTCTTTGTTATCCTGTCTTATGTCTTCATCCTTCAGGCAGTTCTCCAGCTTGCCTCTCAGGAGG
CCCGCTACAAGGCATTTGGGACATGTGTGTCTCACATAGGTGCCATCCTGTCCACCTACACTCCAGTA
GTCATCTCTTCAGTCATGCACCGTGTAGCCCGCCATGCTGCCCCTCGTGTCCACATACTCCTTGCTAT
TTTCTATCTCCTTTTCCCACCCATGGTCAATCCTATCATATATGGAGTCAAGACCAAGCAGATTCGTG
AGTATGTGCTCAGTCTATTCCAGAGAAAGAACATGCTCGAG

NOV53j, CG56144-05

| | | | |
|---|---|---|---|
| Protein Sequence | SEQ ID NO: 828 | 259 aa | MW at 29282.9kD |

ALHEPMYLFLAMLATIDLVLSSTTLPKNLAIFWFRDQEINFFACLVQMFFLHSFSIMESAVLLAMAFD
RYVAICKPLHYTTVLTGSLITKIGMAAVARAVTLMTPLPFLLRRFHYCRGPVIAHCYCEHMAVVRLAC
GDTSFNNIYGIAVAMFIVVLDLLFVILSYVFILQAVLQLASQEARYKAFGTCVSHIGAILSTYTPVVI
SSVMHRVARHAAPRVHILLAIFYLLFPPMVNPIEYGVKTKQIREYVLSLFQRKNM

| | | |
|---|---|---|
| NOV53k, CG56144-06 | SEQ ID NO: 829 | 789 bp |

| | | |
|---|---|---|
| DNA Sequence | ORF Start: at 7 | ORF Stop: at 784 |

GGATCCGCCCTCCATGAACCCATGTACCTCTTTCTGGCCATGTTGGCAACCATTGACTTGGTTCTTTC
TTCTACAACGCTGCCCAAAATGCTTGCCATATTCTGGTTCAGGGATCAGGAGATCAACTTCTTTGCCT
GTCTGGTCCAGATGTTCTTCCTTCACTCCTTCTCCATCATGGAGTCAGCAGTGCTGCTGGCCATGGCC
TTTGACCGCTATGTGGCCATCTGCAAGCCATTGCACTACACGACGGTCCTGACTGGGTCCCTCATCAC
CAAGATTGGCATGGCTGCTGTGGCCCGGGCTGTGACACTAATGACTCCACTCCCCTTCCTGCTCAGAC
GCTTCCACTACTGCCGAGGCCCAGTGATTGCCCATTGCTACTGTGAACACATGGCTGTGGTAAGGCTG
GCGTGTGGGGACACTAGCTTCAACAATATCTATGGCATTGCTGTGGCCATGTTTATTGTGGTGTTGGA
CCTGGTCTTTGTTATCCTGTCTTATGTCTTCATCCTTCAGGCAGTTCTCCAGCTTGCCTCTCAGGAGG
CCCGCTACAAGGCATTTGGGACATGTGTGTCTCACATAGGTGCCATCCTGTCCACCTACACTCCAGTA
GTCATCTCTTCAGTCATGCACCGTGTAGCCCGCCATGCTGCCCCTCGTGTCCACATACTCCTTGCTAT
TTTCTATCTCCTTTTCCCACCCATGGTCAATCCTATCATATATGGAGTCAAGACCAAGCAGATTCGTG
AGTATGTGCTCAGTCTATTCCAGAGAAAGAACATGCTCGAG

NOV53k, CG56144-06

| | | | |
|---|---|---|---|
| Protein Sequence | SEQ ID NO: 830 | 259 aa | MW at 29282.91W |

ALHEPMYLFLAMLATIDLVLSSTTLPKMLAIFWFRDQEINFFACLVQMFFLHSFSIMESAVLLAMAFD
RYVAICKPLHYTTVLTGSLITKIGMAAVARAVTLMTPLPFLLRRFHYCRGPVIAHCYCEHMAVVRLAC
GDTSFNNIYGIAVAMFIVVLDLLFVILSYVFILQAVLQLASQEARYKAFGTCVSHIGAILSTYTPVVI
SSVNHRVARHAAPRVHILLAIFYLLFPPMVNPIIYGVKTKQIREYVLSLFQRKNM

A ClustalW comparison of the above protein sequences yields the following sequence alignment shown in Table 53B.

TABLE 53B
Comparison of the NOV53 protein sequences.

| | |
|---|---|
| NOV53a | ---MTRRFPGAMLPSNITSTHPAVFLLVGIPGLEHLHAWISIPFCFAYTLALLGNCTLLF |
| NOV53b | GSTMTRRFPGAMLPSNITSTHPAVFLLVGIPGLEHLHAWISIPFCFAYTLALLGNCTLLF |
| NOV53c | GSTMTRRFPGANLPSNITSTHPAVFLLVGIPGLEHLHAWISIPFCFAYTLALLGNCTLLF |
| NOV53d | GSTKTRRFPGAMLPSNITSTHPAVFLLVGIPGLEHLHAWISIPFCFAYTLALLGNCTLLF |
| NOV53e | GSTMTRRFPGAMLPSNITSTHPAVFLLVGIPGLEHLHAWISIPFCFAYTLALLGNCTLLF |
| NOV53f | GSTMTRRFPGAMLPSNITSTHPAVFLLVGIPGLENLHAWISIPLCFAYTLALLGNCTLLF |
| NOV53g | ---MTRRFPGAMLPSNITSTHPAVFLLVGIPGLEHLHAWISIPFCFAYTLALLGNCTLLF |
| NOV53h | ------------------------------------------------------------ |
| NOV53i | ------------------------------------------------------------ |
| NOV53j | ------------------------------------------------------------ |
| NOV53k | ------------------------------------------------------------ |
| NOV53a | IIQADAALHEPMYLFLAMLATIDLVLSSTTLPKMLAIFWFRDQEINFFACLVQMFFLHSF |
| NOV53b | IIQADAALHEPMYLFLAMLATIDLVLSSTTLPKMLAIFWFRDQEINFFACLVQMFFLHSF |
| NOV53c | IIQADAALHEPMYLFLAMLATIDLVLSSTTLPKMLAIFWFRDQEINFFACLVQMFFLHSF |
| NOV53d | IIQADAALHEPMYLFLAMLATIDLVLSSTTLPKMLAIFWFRDQEINFFACLVQMFFLHSF |
| NOV53e | IIRAnAALHEPMYLFLAMLATIDLVLSSTTLPKMLAIFWFRDQEINFFACLVQMFFLHSF |
| NOV53f | IIQADAALHEPMYLFLAMLATIDLVLSSTTLPKMLAIFWFRDQEINFFACLVQMFFLHSF |
| NOV53g | IIQADAALHEPMYLFLANLATIDLVLSSTTLPKMLAIFWFRDQEINFFACLVQMFFLHSF |
| NOV53h | ------ALHEPMYLFLAMLATIDLVLSSTTLPKMLAIFWFRDQEINFFACLVQMFFLHSF |
| NOV53i | ------ALHEPMYLFLAMLATIDLVLSSTTLPKMLAIFWFRDQEINFFACLVQMFFLHSF |
| NOV53j | ------ALHEPMYLFLAMLATIDLVLSSTTLPKMLAIFWFRDQEINFFACLVQMFFLHSF |
| NOV53k | ------ALHEPMYLFLAMLATIDLVLSSTTLPKMLAIFWFRDQEINFFACLVQMFFLHSF |
| NOV53a | SIMESAVLLAMAFDRYVAICKPLHYTTVLTGSLITKIGMAAVARAVTLMTPLPFLLRRFH |
| NOV53b | SIMESAVLLAMAFDRYVAICKPLHYTTVLTGSLITKIGMAAVARAVTLMTPLPFLLRRFH |
| NOV53c | SIMESAVLLAMAFDRYVAICKPLHYTTVLTGSLITKIGMAAVARAVTLMTPLPFLLRRFH |
| NOV53d | SIMESAVLLAMAFDRYVAICKPLHYTTVLTGSLITKIGMAAVARAVTLMTPLPFLLRRFH |
| NOV53e | SIMESAVLLAMAFDRYVAICKPLHYTTVLTGSLITKIGMAAVAWAVTLMTPLPFLLRRFH |
| NOV53f | SIMESAVLLAMAFDRYVAICKPLHYTTVLTGSLITKIGMAAVARAVTLMTPLPFLLRRFH |
| NOV53g | SIMESAVLLAMAFDRYVAICKPLHYTTVLTGSLITKIGMAAVARAVTLMTPLPFLLRRFH |
| NOV53h | SINESAVLLAMAFDRYVAICKPLHYTTVLTGSLITKIGMAAVAWAVTLMTPLPFLLRRFH |
| NOV53i | SIMESAVLLAMAFDRYVAICKPLHYTTVLTGSLITKIGMAAVAWAVTLMTPLPFLLRRFH |
| NOV53j | SIMESAVLLAMAFDRYVAICKPLHYTTVLTGSLITKIGMAAVARAVTLMTPLPFLLRRFH |
| NOV53k | SIMESAVLLAMAFDRYVAICKPLHYTTVLTGSLITKIGMAAVARAVTLMTPLPFLLRRFH |
| NOV53a | YCRGPVIAHCYCEHMAVVRLACGDTSFNNIYGIAVAMFSVVLDLLFVILSYVFILQAVLQ |
| NOV53b | YCRGPVIAHCYCEHMAVVRLACGDTSFNNIYGIAVAMFIVVLDLLFVILSYVFILQAVLQ |
| NOV53c | YCRGPVIAHCYCEHMAVVRLACGDTSFNNIYGIAVAMFIVVLDLLFVILSYVFILQAVLQ |
| NOV53d | YCRDPVIAHCYCEHMAVVRLACGDTSFNNIYGIAVAMFIVVLDLLFVILSYVFILQAVLQ |
| NOV53e | YCRGPVIAHCYCEHMAVVRLACGDTSFNNIYGIAVAMFIVVLDLLFVILSYVFILQAVLQ |
| NOV53f | YCRGPVIAHCYCEHMAVVRLACGDTSFNNIYGIAVAMFIVVLDLLFVILSYVFILQAVLQ |
| NOV53g | YCRGPVIAHCYCEHMAVVRLACGDTSFNNIYGIAVAMFIVVLDLLFVILSYVFILQAVLQ |
| NOV53h | YCRGPVIAHCYCEHMAVVRLACGDTSFNNIYGIAVAMSIVVLDLLFVILSYVFILQAVLQ |
| NOV53i | YCRGPVIAHCYCEHMAVVRLACGDTSFNNIYGIAVAMSIVVLDLLFVILSYVFILQAVLQ |
| NOV53j | YCRGPVIAHCYCEHMAVVRLACGDTSFNNIYGIAVAMFIVVLDLLFVILSYVFILQAVLQ |
| NOV53k | YCRGPVIAHCYCEHMAVVRLACGDTSFNNIYGIAVAMFIVVLDLLFVILSYVFILQAVLQ |
| NOV53a | LASQEARYKAFGTCVSHIGAILSTYTPVVISSVMHRVARHAAPRVHILLAIFYLLFPPMV |
| NOV53b | LASQEARYKAFGTCVSHIGAILSTYTPVVISSVMHRVARHAAPRVHILLAIFYLLFPPMV |
| NOV53c | LASQEARYKAFGTCASHIGAILSTYTPVVISSVNHRVARHAAPRVHILLAIFYLLFPPMV |
| NOV53d | LASQEARYKAFGTCVSHIGTILSTYTPVVISSVMHRVARHAAPRVHILLAIFYLLFPPMV |
| NOV53e | LASQEARYKAFGTCVSHIGAILSTYTPVVISSVMHRVARHAAPRVHILLAIFYLLFPPMV |
| NOV53f | LASQEARYKAFGTCVSHIGAILSTYTPVVISSVMHRVARHAAPRVHILLAIFYLLFPPMV |
| NOV53g | LASQEARYKAFGTCVSHIGAILSTYTPVVISSVMHRVARHAAPRVHILLAIFYLLFPPMV |
| NOV53h | LASQEARYKAFGTCVSHIGAILSTYTPVVISSVMHRVARHAAPRVHILLAIFYLLFPPMV |
| NOV53i | LASQEARYKAFGTCVSHIGAILSTYTPVVISSVNHRVARHAAPRVHILLAIFYLLFPPMV |
| NOV53j | LASQEARYKAFGTCVSHIGAILSTYTPVVISSVMHRVARHAAPRVHILLAIFYLLFPPMV |
| NOV53k | LASQEARYKAFGTCVSHIGAILSTYTPVVISSVNHRVARHAAPRVHILLAIFYLLFPPMV |
| NOV53a | NPIIYGVKTKQIREYVLSLFQRKNM-- |
| NOV53b | NPIIYGVKTKQIREYVLSLFQRKNMLE |
| NOV53c | NPIIYGVKTKQIREYVLSLFQRKNMLE |
| NOV53d | NPIIYGVKTKQIREYVLSLFQRKNMLE |
| NOV53e | NPIIYGVKTKQIREYVLSLFQRKNMLE |
| NOV53f | NPIIYGVKTKQIREYVLSLFQRKNMLE |
| NOV53g | NPIIYGVKTKQIREYVLSLFQRKNM-- |
| NOV53h | NPIIYGVKTKQIREYVLSLFQRKNM-- |
| NOV53i | NPIIYGVKTKQIREYVLSLFQRKNM-- |
| NOV53j | NPIIYGVKTKQIREYVLSLFQRKNM-- |
| NOV53k | NPIIYGVKTKQIREYVLSLFQRKNM-- |
| NOV53a | (SEQ ID NO: 810) |
| NOV53b | (SEQ ID NO: 812) |
| NOV53c | (SEQ ID NO: 814) |
| NOV53d | (SEQ ID NO: 816) |
| NOV53e | (SEQ ID NO: 818) |
| NOV53f | (SEQ ID NO: 820) |
| NOV53g | (SEQ ID NO: 822) |
| NOV53h | (SEQ ID NO: 824) |
| NOV53i | (SEQ ID NO: 826) |
| NOV53j | (SEQ ID NO: 828) |
| NOV53k | (SEQ ID NO: 830) |

Further analysis of the NOV53a protein yielded the following properties shown in Table 53C.

TABLE 53C
Protein Sequence Properties NOV53a

| | |
|---|---|
| SignalP analysis: | Cleavage site between residues 64 and 65 |
| PSORT II analysis: | |

| | |
|---|---|
| PSG: | a new signal peptide prediction method |
| | N-region: length 4; pos.chg 2; neg.chg 0 |
| | H-region: length 26; peak value 10.58 |
| | PSG score: 6.18 |
| GvH: | von Heijne's method for signal seq. recognition |
| | GvH score (threshold: −2.1): −3.34 |
| | possible cleavage site: between 61 and 62 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 7 | | |
| | INTEGRAL | Likelihood = −3.29 | Transmembrane 43-59 |
| | INTEGRAL | Likelihood = −2.07 | Transmembrane 69-85 |
| | INTEGRAL | Likelihood = −1.70 | Transmembrane 104-120 |
| | INTEGRAL | Likelihood = 0.26 | Transmembrane 157-173 |
| | INTEGRAL | Likelihood = −11.09 | Transmembrane 210-226 |
| | INTEGRAL | Likelihood = 0.21 | Transmembrane 255-271 |
| | INTEGRAL | Likelihood = −3.03 | Transmembrane 284-300 |

| | |
|---|---|
| PERIPHERAL | Likelihood = 1.54 (at 21) |

| | ALOM score: −11.09 (number of TMSs: 7) |
|---|---|
| MTOP: | Prediction of membrane topology (Hartmann et al.) |
| | Center position for calculation: 50 |
| | Charge difference: −1.5 C(−1.5) − N(0.0) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 12.17 |
| Hyd Moment(95): | 11.23 | G content: | 3 |
| D/E content: | 1 | S/T content: | 5 |
| Score: −1.65 | | | |

Gavel: prediction of cleavage sites for mitochondrial preseq

R-2 motif at 14 RRF|PG

NUCDISC: discrimination of nuclear localization signals

pat4: none
pat7: none
bipartite: none
content of basic residues: 6.8%
NLS Score: −0.47

KDEL: ER retention motif in the C-terminus: none
ER Membrane Retention Signals:

XXRR-like motif in the N-terminus: TRRF
KKXX-like motif in the C-terminus: QRKN

SKL: peroxisomal targeting signal in the C-terminus: none
PTS2: 2nd peroxisomal targeting signal: none
VAC: possible vacuolar targeting motif: found

TLPK at 87

RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

77.8%: endoplasmic reticulum
11.1%: nuclear
11.1%: mitochondrial

>> prediction for CG56144-01 is end (k = 9)

A search of the NOV53a protein against the Geneseq database, a proprietary database that contains sequences published in patents and patent publication, yielded several homologous proteins shown in Table 53D.

TABLE 53D
Geneseq Results for NOV53a

| | | NOV53a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE11904 | Human G-protein coupled | 1 . . . 322 | 318/322 (98%) | 0.0 |
| | receptor 10b (GPCR10b) protein - | 1 . . . 322 | 321/322 (98%) | |
| | Homo sapiens, 322 aa. | | | |
| | [WO200181378-A2, 01-NOV- | | | |
| | 2001] | | | |
| AAE11903 | Human G-protein coupled | 1 . . . 322 | 321/322 (99%) | 0.0 |
| | receptor 10a (GPCR10a) protein - | 1 . . . 321 | 321/322 (99%) | |
| | Homo sapiens, 321 aa. | | | |
| | [WO200181378-A2, 01-NOV- | | | |
| | 2001] | | | |
| AAU85178 | G-coupled olfactory receptor #39 - | 9 . . . 322 | 314/314 (100%) | e−180 |
| | Homo sapiens, 314 aa. | 1 . . . 314 | 314/314 (100%) | |
| | [WO200198526-A2, 27-DEC- | | | |
| | 2001] | | | |
| ABP95675 | Human GPCR polypeptide SEQ | 9 . . . 322 | 314/314 (100%) | e−180 |
| | ID NO 160 - Homo sapiens, 314 | 1 . . . 314 | 314/314 (100%) | |
| | aa. [WO200216548-A2, 28-FEB- | | | |
| | 2002] | | | |
| AAU24558 | Human olfactory receptor | 9 . . . 322 | 314/314 (100%) | e−180 |
| | AOLFR45 - Homo sapiens, 314 | 1 . . . 314 | 314/314 (100%) | |
| | aa. [WO200168805-A2, 20-SEP- | | | |
| | 2001] | | | |

In a BLAST search of public sequence databases, the NOV53a protein was found to have homology to the proteins shown in the BLASTP data in Table 53E.

TABLE 53E
Public BLASTP Results for NOV53a

| | | NOV53a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NGK4 | Seven transmembrane helix | 9 . . . 322 | 311/314 (99%) | e−178 |
| | receptor - Homo sapiens | 1 . . . 314 | 312/314 (99%) | |
| | (Human), 314 aa. | | | |
| Q8NGK3 | Seven transmembrane helix | 9 . . . 322 | 283/314 (90%) | e−161 |
| | receptor - Homo sapiens | 1 . . . 314 | 290/314 (92%) | |
| | (Human), 314 aa. | | | |
| Q8VH04 | Olfactory receptor MOR28-1 - | 9 . . . 322 | 273/314 (86%) | e−157 |
| | Mus musculus (Mouse), 317 aa. | 1 . . . 314 | 291/314 (91%) | |
| CAD20423 | Sequence 1 from Patent | 9 . . . 316 | 276/308 (89%) | e−154 |
| | WO0179295 Homo sapiens | 1 . . . 307 | 287/308 (92%) | |
| | (Human), 316 aa. | | | |
| Q8VG23 | Olfactory receptor MOR29-1 - | 9 . . . 319 | 172/311 (55%) | 3e−96 |
| | Mus musculus (Mouse), 315 aa. | 1 . . . 310 | 224/311 (71%) | |

PFam analysis indicates that the NOV53a protein contains the domains shown in the Table 53F.

TABLE 53F
Domain Analysis of NOV53a

| | | Identities/ | |
|---|---|---|---|
| | | Similarities | |
| Pfam | NOV53a | for the Matched | Expect |
| Domain | Match Region | Region | Value |
| 7tm_1 | 51 . . . 302 | 50/277 (18%) | 4.5e−13 |
| | | 163/277 (59%) | |

### Example 54

The NOV54 clone was analyzed, and the nucleotide and encoded polypeptide sequences are shown in Table 54A.

TABLE 54A
NOV54 Sequence Analysis

| | | |
|---|---|---|
| NOV54a, CG56146-02 | SEQ ID NO: 831 | 807 bp |

| | | |
|---|---|---|
| DNA Sequence | ORF Start: ATG at 1 | ORF Stop: end of sequence |

ATGATTCTGGACCATCGTCTCCACATGGCAATGTACTTTTTCCTCCGACATTTGTCCTTCTTAGACCT
GTGTCTCATTTCTGACACAGTCCCCAAATCCATCCTCAACTCTGTCGCCTCCACTGACTCCATCTCCT
TCCTGGGGTGTGTGTTGCAGCTCTTCTTGGTGGTACTGCTGGCTGGATCAGAGATTGGCATCCTTACT
GCCATGTCCTATGACCGCTATGCTGCCATCTGCTGCCCCCTACACTGTGAGGCTGTCATGAGCAGAGG
GCTCTGTGTCCAGTTGATGGCTCTGTCCTGGCTCAACAGAGGGGCCTTGGGACTCTTGTACACAGCTG
GAACATTCTCTCTGAATTTTTATGGCTCTGATGAGCTACATCAGTTCTTCTGCGATGTCCCTGCCCTA
CTAAAGCTCACTTGTTCTAAAGAACATGCCATCATTAGTGTCAGTGTGGCCATTGGGGTCTGTTATGC
ATTTTCATGTTTAGTTTGCATTGTAGTTTCCTATGTGTACATTTTCTCTGCTGTGTTAAGGATATCAC
AGAGACAGAGACAATCCAAAGCCTTTTCCAACTGTGTGCCTCACCTCATTGTTGTCACTGTGTTTCTT
GTAACAGGTGCTGTTGCTTATTTAAAGCCAGGGTCTGATGCACCTTCTATTCTAGACTTGCTGGTGTC
TGTGTTCTATTCTGTCGCACCTCCAACCTTGAACCCTGTTATCTACTGTCTGAAGAACAAGGACATTA
AATCCGCTCTGAGTAAAGTCCTGTGGAATGTTAGAAGCAGTGGGGTAATGAAAGATGAC

NOV54a, CG56146-02

| | | | |
|---|---|---|---|
| Protein Sequence | SEQ ID NO: 832 | 269 aa | MW at 29515.5kD |

MILDHRLHMAHYFFLRHLSFLDLCLISDTVPKSILNSVASTDSISFLGCVLQLFLVVLLAGSEIGILT
AMSYDRYAAICCPLHCEAVMSRGLCVQLMALSWLNRGALGLLYTAGTFSLNFYGSDELHQFFCDVPAL
LKLTCSKEHAIISVSVAIGVCYAFSCLVCIVVSYVYIFSAVLRISQRQRQSKAFSNCVPHLIVVTVFL
VTGAVAYLKPGSDAPSILDLLVSVFYSVAPPTLNPVIYCLKKNDIKSALSKVLNNVRSSGVMKDD

| | | |
|---|---|---|
| NOV54b, CG56146-01 | SEQ ID NO: 833 | 995 bp |

| | | |
|---|---|---|
| DNA Sequence | ORF Start: ATG at 21 | ORF Stop: TAA at 966 |

GGCGCTTATAATTTTGAACTATGACCAATCAGACACAGATGATGGAATTCTTGCTTGTGAGATTTACT
GAGAATTGGGTGCTCCTGAGGCTGCATGCTTTGCTCTTCTCACTGATCTACCTCACGGCTGTGCTGAT
GAATTTAGTCATCATTCTCCTCATGATTCTGGACCATCGTCTCCACATGGCAATGTACTTTTTCCTCC
GACATTTGTCCTTCTTAGACCTGTGTCTCATTTCTGCCACAGTCCCCAAATCCATCCTCAACTCTGTC
GCCTCCACTGACTCCATCTCCTTCCTGGGGTGTGTGTTGCAGCTCTTCTTGGTGGTACTGCTGGCTGG
ATCAGAGATTGGCATCCTTACTGCCATGTCCTATGACCGCTATGCTGCCATCTGCTGCCCCCTACACT
GTGAGGCTGTCATGAGCAGAGGGCTCTGTGTCCAGTTGATGGCTCTGTCCTGGCTCAACAGAGGGGCC
TTGGGACTCTTGTACACAGCTGGAACATTCTCTCTGAATTTTTATGGCTCTGATGAGCTACATCAGTT
CTTCTGCGATGTCCCTGCCCTACTAAAGCTCACTTGTTCTAAAGAACATGCCATCATTAGTGTCAGTG
TGGCCATTGGGGTCTGTTATGCATTTTCATGTTTAGTTTGCATTGTAGTTTCCTATGTGTACATTTTC
TCTGCTGTGTTAAGGATATCACAGAGACAGAGACAATCCAAAGCCTTTTCCAACTGTGTGCCTCACCT
CATTGTTGTCACTGTGTTTCTTGTAACAGGTGCTGTTGCTTATTTAAAGCCAGGGTCTGATGCACCTT
CTATTCTAGACTTGCTGGTGTCTGTGTTCTATTCTGTCGCACCTCCAACCTTGAACCCTGTTATCTAC
TGTCTGAAGAACAAGGACATTAAATCCGCTCTGAGTAAAGTCCTGTGGAATGTTAGAAGCAGTGGGGT
AATGAAAGATGACTAAAGTTGAAGATGGGAAGTACTTTTTTTG

NOV54b, CG56146-01

| | | | |
|---|---|---|---|
| Protein Sequence | SEQ ID NO: 834 | 315 aa | MW at 34934.2kD |

MTNQTQMMEFLLVRFTENWVLLRLHALLFSLIYLTAVLMNLVIILLMILDHRLHMAMYFFLRHLSFLD
LCLISATVPKSILNSVASTDSISFLGCVLQLFLVVLLAGSEIGILTANSYDRYAAICCPLHCEAVMSR
GLCVQLMALSWLNRGALGLLYTAGTFSLNFYGSDELHQFFCDVPALLKLTCSKEHAIISVSVAIGVCY
AFSCLVCIVVSYVYIFSAVLRISQRQRQSKAFSNCVPHLIVVTVFLVTGAVAYLKPGSDAPSILDLLV
SVFYSVAPPTLNPVIYCLKNKDIKSALSKVLWNVRSSGVMKDD

| | | |
|---|---|---|
| NOV54c, 170646057 | SEQ ID NO: 835 | 819 bp |

| | | |
|---|---|---|
| DNA Sequence | ORF Start: at 1 | ORF Stop: end of sequence |

GGATCCATGATTCTGGACCATCGTCTCCACATGGCAATGTACTTTTTCCTCCGACATTTGTCCTTCTT
AGACCTGTGTCTCATTTCTGCCACAGTCCCCAAATCCATCCTCAACTCTGTCGCCTCCACTGACTCCA
TCTCCTTCCTGGGGTGTGTGTTGCAGCTCTTCTTGGTGGTACTGCTGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTCTGAATTTTTATGGCTCTGATGAGCTACATCAGTTCTTCTGCGATGTCCCT
GCCCTACTAAAGCTCACTTGTTCTAAAGAACATGCCATCATTAGTGTCAGTGTGGCCATTGGGGTCTG
TTATGCATTTTCATGTTTAGTTTGCATTGTAGTTTCCTATGTGTACATTTTCTCTGCTGTGTTAAGGA
TATCACAGAGACAGAGACAATCCAAAGCCTTTTCCAACTGTGTGCCTCACCTCATTGTTGTCACTGTG
TTTCTTGTAACAGGTGCTGTTGCTTATTTAAAGCCAGGGTCTGATACACCTTCTATTCTAGACTTGCT
GGTGTCTGTGTTCTATTCTGTCGCACCTCCAACCTTGAACCCTGTTATCTACTGTCTGAAGAACAAGG
ACATTAAATCCGCTCTGAGTAAAGTCCTGTGGAATGTTAGAAGCAGTGGGGTAATGAAAGATGACCTC
GAG

NOV54c, 170646057

| | | | |
|---|---|---|---|
| Protein Sequence | SEQ ID NO: 836 | 273 aa | MW at 29888.0kD |

GSMILDHRLHMAMYFFLRHLSFLDLCLISATVPKSILNSVASTDSISFLGCVLQLFLVVLLAGSEIGI
LTAMSYDRYAAICCPLHCEAVMSRGLCVQLMALSWLNRGALGLLYTAGTFSLNFYGSDELHQFFCDVP
ALLKLTCSKEHAIISVSVAIGVCYAFSCLVCIVVSYVYIFSAVLRISQRQRQSKAFSNCVPHLIVVTV
FLVTGAVAYLKPGSDTPSILDLLVSVFYSVAPPTLNPVIYCLKNKDIKSALSKVLWNVRSSGVMKDDL
E

| | | |
|---|---|---|
| NOV54d, 170646049 | SEQ ID NO: 837 | 819 bp |

| | | |
|---|---|---|
| DNA Sequence | ORF Start: at 1 | ORF Stop: end of sequence |

GGATCCATGATTCTGGACCATCGTCTCCACATGGCAATGTACTTTTTCCTCCGACATTTGTCCTTCTT
AGACCTGTGTCTCATTTCTGCCGCAGTCCCCAAATCCATCCTCAACTCTGTCGCCTCCACTGACTCCA
TCTCCTTCCTGGGGTGTGTGTTGCAGCTCTTCTTGGTGGTACTGCTGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTCTGAATTTTTATGGCTCTGATGAGCTACATCAGTTCTTCTGCGATGTCCCT
GCCCTACTAAAGCTCACTTGTTCTAAAGAACATGCCATCATTAGTGTCAGTGTGGCCATTGGGGTCTG
TTATGCATTTTCATGTTTAGTTTGCATTGTAGTTTCCTATGTGTACATTTTCTCTGCTGTGTTAAGGA
TATCACAGAGACAGAGACAATCCAAAGCCTTTTCCAACTGTGTGCCTCACCTCATTGTTGTCACTGTG
TTTCTTGTAACAGGTGCTGTTGCTTATTTAAAGCCAGGGTCTGATGCACCTTCTATTCTAGACTTGCT
GGTGTCTGTGTTCTATTCTGTCGCACCTCCAACCTTGAACCCTGTTATCTACTGTCTGAAGAACAAGG
ACATTAAATCCGCTCTGAGTAAAGTCCTGTGGAATGTTAGAAGCAGTGGGGTAATGAAAGATGACCTC
GAG

NOV54d, 170646049

| | | | |
|---|---|---|---|
| Protein Sequence | SEQ ID NO: 838 | 273 aa | MW at 29827.9kD |

GSMILDHRLHMAMYFFLRHLSFLDLCLISAAVPKSILNSVASTDSISFLGCVLQLFLVVLLAGSEIGI
LTAMSYDRYAAICCPLHCEAVMSRGLCVQLMALSWLNRGALGLLYTAGTFSLNFYGSDELHQFFCDVP
ALLKLTCSKEHAIISVSVAIGVCYAFSCLVCIVVSYVYIFSAVLRISQRQRQSKAFSNCVPHLIVVTV
FLVTGAVAYLKPGSDAPSILDLLVSVFYSVAPPTLNPVIYCLKNKDIKSALSKVLWNVRSSGVMKDDL
E

| | | |
|---|---|---|
| NOV54e, 170646053 | SEQ ID NO: 839 | 819 bp |

| | | |
|---|---|---|
| DNA Sequence | ORF Start: at 1 | ORF Stop: end of sequence |

GGATCCATGATTCTGGACCATCGTCTCCACATGGCAATGTACTTTTTCCTCCGACATTTGTCCTTCTT
AGACCTGTGTCTCATTTCTGCCACAGTCCCCAAATCCATCCTCAACTCTGTCGCCTCCACTGACTCCA
TCTCCTTCCTGGGGTGTGTGTTGCAGCTCTTCTTGGTGGTACTGCCGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTCTGAATTTTTATGGCTCTGATGAGCTACATCAGTTCTTCTGCGATGTCCCT
GCCCTACTAAAGCTCACTTGTTCTAAAGAACATGCCATCATTAGTGTCAGTGGGGCCATTGGGGTCTG
TTATGCATTTTCATGTTTAGTTTGCATTGTAGTTTCCTATGTGTACATTTTCTCTGCTGTGTTAAGGA
TATCACAGAGACAGAGACAATCCAAAGCCTTTTCCAACTGTGTGCCTCACCTCATTGTTGTCACTGTG
TTTCTTGTAACAGGTGCTGTTGCTTATTTAAAGCCAGGGTCTGATGCACCTTCTATTCTAGACTTGCT
GGTGTCTGTGTTCTATTCTGTCGCACCTCCAACCTTGAACCCTGTTATCTACTGTCTGAAGAACAAGG
ACATTAAATCCGCTCTGAGTAAAGTCCTGTGGAATGTTAGAAGCAGTGGGGTAATGAAAGATGACCTC
GAG

NOV54e, 170646053

| | | | |
|---|---|---|---|
| Protein Sequence | SEQ ID NO: 840 | 273 aa | MW at 29799.8kD |

GSMILDHRLHMAMYFFLRHLSFLDLCLISATVPKSILNSVASTDSISFLGCVLQLFLVVLPAGSEIGI
LTAMSYDRYAAICCPLHCEAVMSRGLCVQLMALSWLNRGALGLLYTAGTFSLNFYGSDELHQFFCDVP
ALLKLTCSKEHAIISVSGAIGVCYAFSCLVCIVVSYVYIFSAVLRISQRQRQSKAFSNCVPHLIVVTV
FLVTGAVAYLKPGSDAPSILDLLVSVFYSVAPPTLNPVIYCLKNKDIKSALSKVLWNVRSSGVMKDDL
E

| | | |
|---|---|---|
| NOV54f, 174307717 | SEQ ID NO: 841 | 960 bp |

| | | |
|---|---|---|
| DNA Sequence | ORF Start: at 1 | ORF Stop: end of sequence |

GGATCCACCATGACCAATCAGACACAGATGATGGAATTCTTGCTTGTGAGATTTACTGAGAATTGGGT
GCTCCTGAGGCTGCATGCTTTGCTCTTCTCACTGATCTACCTCACGGCTGTGCTGATGAATTTAGTCA
TCATTCTCCTCATGATTCTGGACCATCGTCTCCACATGGCAATGTACTTTTTCCTCCGACATTTGTCC
TTCTTAGACCTGTGTCTCATTTCTGCCACAGTCCCGAAATCCATCCTCAACTCTGTCGCCTCCACTGA
CTCCATCTCCTTCCTGGGGTGTGTGTTGCAGCTCTTCTTGGTGGTACTGCTGGCTGGATCAGAGATTG
GCATCCCTACTGCCATGTCCTATGACCGCTATGCTGCCATCTGCTGCCCCCTACACTGTAAGGCTGTC
ATGAGCAGAGGGCTCTGTGTCCAGTTGATGGCTCTGTCCTGGCTCAACAGAGGGGCCTTGGGACTCTT
GTACACAGCTGGAACATTCTCTCTGAATTTTTATGGCTCTGATGAGCTACATCAGTTCTTCTGCGATG
TCCCTGCCCTACTAAAGCTCACTTGTTCTAAAGAACATGCCATCATTAGTGTCAGTGTGGCCATTGGG
GTCTGTTATGCATTTTCATGTTTAGTTTGCATTGTAGTTTCCTATGTGTACATTTTCTCTGCTGTGTT
AAGGATATCACAGAGACAGAGACAATCCAAAGCCTTTTCCAACTGTGTGCCTCACCTCATTGTTGTCA
CTGTGTTTCTTGTAACAGGTGCTGTTGCTTATTTAAAGCCAGGGTCTGATGCACCTTCTATTCTAGAC
TTGCTGGTGTCTGTGTTCTATTCTGTCGCACCTCCAACCTTGAACCCTGTTATCTACTGTCTGAAGAA
CAAGGACATTAAATCCGCTCTGAGTAAAGTCCTGTGGAATGTTAGAAGCAGTGGGGTAATGAAAGATG
ACCTCGAG

NOV54f, 174307717

| | | | |
|---|---|---|---|
| Protein Sequence | SEQ ID NO: 842 | 320 aa | MW at 35404.7kD |

GSTMTNQTQMMEFLLVRFTENWVLLRLHALLFSLIYLTAVLNNLVIILLMILDHRLHMAMYFFLRHLS
FLDLCLISATVPKSILNSVASTDSISFLGCVLQLFLVVLLAGSEIGIPTANSYDRYAAICCPLHCKAV
MSRGLCVQLMALSWLNRGALGLLYTAGTFSLNFYGSDELHQFFCDVPALLKLTCSKEHAIISVSVAIG
VCYAFSCLVCIVVSYVYIFSAVLRISQRQRQSKAFSNCVPHLIVVTVFLVTGAVAYLKPGSDAPSILD
LLVSVFYSVAPPTLNPVIYCLKNKDIKSALSKVLWNVRSSGVMKDDLE

| | | |
|---|---|---|
| NOV54g, 168869383 | SEQ ID NO: 843 | 820 bp |

| | | |
|---|---|---|
| DNA Sequence | ORF Start: at 296 | ORF Stop: end of sequence |

GGATCCATGATTCTGGACCATCGTCTCCACATGGCAATGTACTTTTTCCTCCGACATTTGTCCTTCTT
AGACCTGTGTCTCATTTCTGCCACAGTCCCCAAATCCATCCTCAACTCTGTCGCCTCCACTGACTCCA
TCTCCTTCCTGGGGTGTGTGTTGCAGCTCTTCTTGGTGGTACTGCTGGCTGGATCAGAGATTGGCATC
CTTACTGCCATGTCCTATGACCGCTATGCTGCCATCTGCTGCCCCCTACACTGTGAGGCTGTCATGAG
CAGAGGGCTCTGTGTCCAGTTGATGGCTCTGTCCTGGCTCAACAGAGGGGCCTTGGGACTCTTGTACA
CAGCTGGAACATTCTCTTCTGAATTTTTATGGCTCTGATGAGCTACATCAGTTCTTCTGCGATGTCCC
TGCCCTACTAAAGCTCACTTGTTCTAAAGAACATGCCATCATTAGTGTCAGTGTGGCCATTGGGGTCT
GTTATGCATTTTCATGTTTAGTTTGCATTGTAGTTTCCTATGTGTACATTTTCTCTGCTGTGTTAAGG
ATATCACAGAGACAGAGACAATCCAAAGCCTTTTCCAACTGTGTGCCTCACCTCATTGTTGTCACTGT
GTTTCTTGTAACAGGTGCTGTTGCTTATTTAAAGCCAGGGTCTGATGCACCTTCTATTCTAGACTTGC
TGGTGTCTGTGTTCTATTCTGTCGCACCTCCAACCTTGAACCCTGTTATCTACTGTCTGAAGAACAAG
GACATTAAATCCGCTCTGAGTAAAGTCCTGTGGAATGTTAGAAGCAGTGGGGTAATGAAAGATGACCT
CGAG

NOV54g, 168869383

| | | | |
|---|---|---|---|
| Protein Sequence | SEQ ID NO: 844 | 175 aa | MW at 19239.2kD |

WLCPGSTEGPWDSCTQLEHSLLNFYGSDELHQFFCDVPALLKLTCSKEHAIISVSVAIGVCYAFSCLV
CIVVSYVYIFSAVLRISQRQRQSKAFSNCVPHLIVVTVFLVTGAVAYLKPGSDAPSILDLLVSVFYSV
APPTLNPVIYCLKNKDIKSALSKVLWNVRSSGVMKDDLE

| | | |
|---|---|---|
| NOV54h, CG56146-03 | SEQ ID NO: 845 | 945 bp |

| | | |
|---|---|---|
| DNA Sequence | ORF Start: ATG at 1 | ORF Stop: end of sequence |

ATGACCAATCAGACACAGATGATGGAATTCTTGCTTGTGAGATTTACTGAGAATTGGGTGCTCCTGAG
GCTGCATGCTTTGCTCTTCTCACTGATCTACCTCACGGCTGTGCTGATGAATTTAGTCATCATTCTCC
TCATGATTCTGGACCATCGTCTCCACATGGCAATGTACTTTTTCCTCCGACATTTGTCCTTCTTAGAC
CTGTGTCTCATTTCTGCCACAGTCCCCAAATCCATCCTCAACTCTGTCGCCTCCACTGACTCCATCTC
CTTCCTGGGGTGTGTGTTGCAGCTCTTCTTGGTGGTACTGCTGGCTGGATCAGAGATTGGCATCCTTA
CTGCCATGTCCTATGACCGCTATGCTGCCATCTGCTGCCCCCTACACTGTGAGGCTGTCATGAGCAGA
GGGCTCTGTGTCCAGTTGATGGCTCTGTCCTGGCTCAACAGAGGGGCCTTGGGACTCTTGTACACAGC
TGGAACATTCTCTCTGAATTTTTATGGCTCTGATGAGCTACATCAGTTCTTCTGCGATGTCCCTGCCC
TACTAAAGCTCACTTGTTCTAAAGAACATGCCATCATTAGTGTCAGTGTGGCCATTGGGGTCTGTTAT
GCATTTTCATGTTTAGTTTGCATTGTAGTTTCCTATGTGTACATTTTCTCTGCTGTGTTAAGGATATC
ACAGAGACAGAGACAATCCAAAGCCTTTTCCAACTGTGTGCCTCACCTCATTGTTGTCACTGTGTTTC
TTGTAACAGGTGCTGTTGCTTATTTAAAGCCAGGGTCTGATGCACCTTCTATTCTAGACTTGCTGGTG
TCTGTGTTCTATTCTGTCGCACCTCCAACCTTGAACCCTGTTATCTACTGTCTGAAGAACAAGGACAT
TAAATCCGCTCTGAGTAAAGTCCTGTGGAATGTTAGAAGCAGTGGGGTAATGAAAGATGAC

NOV54h, CG56146-03

| | | | |
|---|---|---|---|
| Protein Sequence | SEQ ID NO: 846 | 315 aa | MW at 34934.2kD |

MTNQTQMNEFLLVRFTENWVLLRLHALLFSLIYLTAVLMNLVIILLMILDHRLHMAMYFFLRHLSFLD
LCLISATVPKSILNSVASTDSISFLGCVLQLFLVVLLAGSEIGILTAMSYDRYAAICCPLHCEAVMSR
GLCVQLMALSWLNRGALGLLYTAGTFSLNFYGSDELHQFFCDVPALLKLTCSKEHAIISVSVAIGVCY
AFSCLVCIVVSYVYIFSAVLRISQRQRQSKAFSNCVPHLIVVTVFLVTGAVAYLKPGSDAPSILDLLV
SVFYSVAPPTLNPVIYCLKNKDIKSALSKVLWNVRSSGVMKDD

| | | |
|---|---|---|
| NOV54i, 262939640 | SEQ ID NO: 847 | 952 bp |

| | | |
|---|---|---|
| DNA Sequence | ORF Start: at 3 | ORF Stop: TAG at 942 |

CCAATCAGACACAGATGATGGAATTCTTGCTTGTGAGATTTACTGAGAATTGGGTGCTCCTGAGGCTG
CATGCTTTGCTCTTCTCACTGATCTACCTCACGGCTGTGCTGATGAATTTAGTCATCATTCTCCTCAT
GATTCTGGACCATCGTCTCCACATGGCAATGTACTTTTTCCTCCGACATTTGTCCTTCTTAGACCTGT
GTCTCATTTCTGCCACAGTCCCCAAATCCATCCTCAACTCTGTCGCCTCCACTGACTCCATCTCCTTC
CTGGGGTGTGTGTTGCAGCTCTTCTTGGTGGTACTGCTGGCTGGATCAGAGATTGGCATCCTTACTGC
CATGTCCTATGACCGCTATGCTGCCATCTGCTGCCCCCTACACTGTGAGGCTGTCATGAGCAGAGGGC
TCTGTGTCCAGTTGATGGCTCTGTCCTGGCTCAACAGAGGGGCCTTGGGACTCTTGTACACAGCTGGA
ACATTCTCTCTGAATTTTTATGGCTCTGATGAGCTACATCAGTTCTTCTGCGATGTCCCTGCCCTACT
AAAGCTCACTTGTTCTAAAGAACATGCCATCATTAGTGTCAGTGTGGCCATTGGGGTCTGTTATGCAT
TTTCATGTTTAGTTTGCATTGTAGTTTCCTATGTGTACATTTTCTCTGCTGTGTTAAGGATATCACAG
AGACAGAGACAATCCAAAGCCTTTTCCAACTGTGTGCCTCACCTCATTGTTGTCACTGTGTTTCTTGT
AACAGGTGCTGTTGCTTATTTAAAGCCAGGGTCTGATGCACCTTCTATTCTAGACTTGCTGGTGTCTG
TGTTCTATTCTGTCGCACCTCCAACCTTGAACCCTGTTATCTACTGTCTGAAGAACAAGGACATTAAA
TCCGCTCTGAGTAAAGTCCTGTGGAATGTTAGAAGCAGTGGGGTAATGAAAGATGACTAGCAATTGGG

NOV54i, 262939640

| | | | |
|---|---|---|---|
| Protein Sequence | SEQ ID NO: 848 | 313 aa | MW at 34701.9kD |

NQTQMMEFLLVRFTENWVLLRLHALLFSLIYLTAVLMNLVIILLMILDHRLHMAMYFFLRHLSFLDLC
LISATVPKSILNSVASTDSISFLGCVLQLFLVVLLAGSEIGILTAMSYDRYAAICCPLHCEAVMSRGL
CVQLMALSWLNRGALGLLYTAGTFSLNFYGSDELHQFFCDVPAlLKLTCSKEHAIISVSVAIGVCYAF
SCLVCIVVSYVYIFSAVIRISQRQRQSKAFSNCVPHLIVVTVFLVTGAVAYLKPGSDAPSILDLLVSV
FYSVAPPTLNPVIYCLKNKDIKSALSKVLWNVRSSGVMKDD

| | | |
|---|---|---|
| NOV54j, CG56146-04 | SEQ ID NO: 849 | 60 bp |

| | | |
|---|---|---|
| DNA Sequence | ORF Start: ATG at 1 | ORF Stop: end of sequence |

ATGATGGAATTCTTGCTTGTGAGATTTACTGAGAATTGGGTGCTCCTGAGGCTGCATGCT

NOV54j, CG56146-04

| | | | |
|---|---|---|---|
| Protein Sequence | SEQ ID NO: 850 | 20 aa | MW at 2519.0kD |

MMEFLLVRFTENWVLLRLHA

| | | |
|---|---|---|
| NOV54k, CG56146-05 | SEQ ID NO: 851 | 60 bp |

| | | |
|---|---|---|
| DNA Sequence | ORF Start: at 1 | ORF Stop: end of sequence |

GATGTCCCTGCCCTACTAAAGCTCACTTGTTCTAAAGAACATGCCATCATTAGTGTCAGT

| | | |
|---|---|---|
| NOV54l, CG561146-06 | SEQ ID NO: 853 | 952 bp |

| | | |
|---|---|---|
| DNA Sequence | ORF Start: at 3 | ORF Stop: TAG at 942 |

CCAATCAGACACAGATGATGGAATTCTTGCTTGTGAGATTTACTGAGAATTGGGTGCTCCTGAGGCTG
CATGCTTTGCTCTTCTCACTGATCTACCTCACGGCTGTGCTGATGAATTTAGTCATCATTCTCCTCAT
GATTCTGGACCATCGTCTCCACATGGCAATGTACTTTTTCCTCCGACATTTGTCCTTCTTAGACCTGT
GTCTCATTTCTGCCACAGTCCCCAAATCCATCCTCAACTCTGTCGCCTCCACTGACTCCATCTCCTTC
CTGGGGTGTGTGTTGCAGCTCTTCTTGGTGGTACTGCTGGCTGGATCAGAGATTGGCATCCTTACTGC
CATGTCCTATGACCGCTATGCTGCCATCTGCTGCCCCCTACACTGTGAGGCTGTCATGAGCAGAGGGC
TCTGTGTCCAGTTGATGGCTCTGTCCTGGCTCAACAGAGGGGCCTTGGGACTCTTGTACACAGCTGGA
ACATTCTCTCTGAATTTTTATGGCTCTGATGAGCTACATCAGTTCTTCTGCGATGTCCCTGCCCTACT
AAAGCTCACTTGTTCTAAAGAACATGCCATCATTAGTGTCAGTGTGGCCATTGGGGTCTGTTATGCAT
TTTCATGTTTAGTTTGCATTGTAGTTTCCTATGTGTACATTTTCTCTGCTGTGTTAAGGATATCACAG
AGACAGAGACAATCCAAAGCCTTTTCCAACTGTGTGCCTCACCTCATTGTTGTCACTGTGTTTCTTGT
AACAGGTGCTGTTGCTTATTTAAAGCCAGGGTCTGATGCACCTTCTATTCTAGACTTGCTGGTGTCTG
TGTTCTATTCTGTCGCACCTCCAACCTTGAACCCTGTTATCTACTGTCTGAAGAACAAGGACATTAAA
TCCGCTCTGAGTAAAGTCCTGTGGAATGTTAGAAGCAGTGGGGTAATGAAAGATGACTAGCAATTGGG

NOV54l, CG56146-06

| | | | |
|---|---|---|---|
| Protein Sequence | SEQ ID NO: 854 | 313 aa | MW at 34701.9kD |

NQTQMMEFLLVRFTENWVLLRLHALLFSLIYLTAVLMNLVIILLMILDHRLHMAMYFFLRHLSFLDLC
LISATVPKSILNSVASTDSISFLGCVLQLFLVVLLAGSEIGILTANSYDRYAAICCPLHCEAVMSRGL
CVQLMALSWLNRGALGLLYTAGTFSLNFYGSDELHQFFCDVPALLKLTCSKEHAIISVSVAIGVCYAF
SCLVCIVVSYVYIFSAVLRISQRQRQSKAFSNCVPHLIVVTVFLVTGAVAYLKPGSDAPSILDLLVSV
FYSVAPPTLNPVIYCLKNKDIKSALSKVLWNVRSSGVMKDD

A ClustalW comparison of the above protein sequences yields the following sequence alignment shown in Table 54B.

TABLE 54B
Comparison of the NOV54 protein sequences.

| | |
|---|---|
| NOV54a | -------------------------------------------------MILDHRLHMAM |
| NOV54b | ---MTNQTQMMEFLLVRFTENWVLLRLHALLFSLIYLTAVLMNLVIILLMILDHRLHMAM |
| NOV54c | -----------------------------------------------GSMILDHRLHMAM |
| NOV54d | -----------------------------------------------GSMILDHRLHMAM |
| NOV54e | -----------------------------------------------GSMILDHRLHMAM |
| NOV54f | GSTMTNQTQMMEFLLVRFTENWVLLRLHALLFSLIYLTAVLMNLVIILLMILDHRLHMAM |
| NOV54g | ------------------------------------------------------------ |
| NOV54h | ---MTNQTQMMEFLLVRFTENWVLLRLHALLFSLIYLTAVLMNLVIILLMILDHRLHMAM |
| NOV54i | -----NQTQMMEFLLVRFTENWVLLRLHALLFSLIYLTAVLMNLVIILLMILDHRLHMAM |
| NOV54j | ------------------------------------------------------------ |
| NOV54k | ------------------------------------------------------------ |
| NOV54l | -----NQTQMMEFLLVRFTENWVLLRLHALLFSLIYLTAVLMNLVIILLMILDHRLHMAM |
| NOV54a | YFFLRHLSFLDLCLISDTVPKSILNSVASTDSISFLGCVLQLFLVVLLAGSEIGILTAMS |
| NOV54b | YFFLRHLSFLDLCLISATVPKSILNSVASTDSISFLGCVLQLFLVVLLAGSEIGILTAMS |
| NOV54c | YFFLRHLSFLDLCLISATVPKSILNSVASTDSISFLGCVLQLFLVVLLAGSEIGILTAMS |
| NOV54d | YFFLRHLSFLDLCLISAAVPKSILNSVASTDSISFLGCVLQLFLVVLLAGSEIGILTAMS |
| NOV54e | YFFLRHLSFLDLCLISATVPKSILNSVASTDSISFLGCVLQLFLVVLPAGSEIGILTAMS |
| NOV54f | YFFLRHLSFLDLCLISATVPKSILNSVASTDSISFLGCVLQLFLVVLLAGSEIGIPTAMS |
| NOV54g | ------------------------------------------------------------ |
| NOV54h | YFFLRHLSFLDLCLISATVPKSILNSVASTDSISFLGCVLQLFLVVLLAGSEIGILTAMS |
| NOV54i | YFFLRHLSFLDLCLISATVPKSILNSVASTDSISFLGCVLQLFLVVLLAGSEIGILTAMS |
| NOV54j | ------------------------------------------------------------ |
| NOV54k | ------------------------------------------------------------ |
| NOV54l | YFFLRHLSFLDLCLISATVPKSILNSVASTDSISFLGCVLQLFLVVLLAGSEIGILTAMS |
| NOV54a | YDRYAAICCPLHCEAVMSRGLCVQLMALSWLNRGALGLLYTAGTFSLNFYGSDELHQFFC |
| NOV54b | YDRYAAICCPLHCEAVMSRGLCVQLMALSWLNRGALGLLYTAGTFSLNFYGSDELHQFFC |
| NOV54c | YDRYAAICCPLHCEAVMSRGLCVQLMALSWLNRGALGLLYTAGTFSLNFYGSDELHQFFC |
| NOV54d | YDRYAAICCPLHCEAVMSRGLCVQLMALSWLNRGALGLLYTAGTFSLNFYGSDELHQFFC |
| NOV54e | YDRYAAICCPLHCEAVMSRGLCVQLMALSWLNRGALGLLYTAGTFSLNFYGSDELHQFFC |
| NOV54f | YDRYAAICCPLHCKAVMSRGLCVQLMALSWLNRGALGLLYTAGTFSLNFYGSDELHQFFC |
| NOV54g | ------WLCPGSTEGPWDS--CTQLEHS-----------------LLNFYGSDELHQFFC |
| NOV54h | YDRYAAICCPLHCEAVMSRGLCVQLMALSWLNRGALGLLYTAGTFSLNFYGSDELHQFFC |
| NOV54i | YDRYAAICCPLHCEAVMSRGLCVQLMALSWLNRGALGLLYTAGTFSLNFYGSDELHQFFC |
| NOV54j | --------------------------------------------------MMEFLLVRFT |
| NOV54k | ------------------------------------------------------------ |
| NOV54l | YDRYAAICCPLHCEAVMSRGLCVQLMALSWLNRGALGLLYTAGTFSLNFYGSDELHQFFC |
| NOV54a | DVPALLKLTCSKEHAIISVSVAIGVCYAFSCLVCIVVSYVYIFSAVLRISQRQRQSKAFS |
| NOV54b | DVPALLKLTCSKEHAIISVSVAIGVCYAFSCLVCIVVSYVYIFSAVLRISQRQRQSKAFS |
| NOV54c | DVPALLKLTCSKEHAIISVSVAIGVCYAFSCLVCIVVSYVYIFSAVLRISQRQRQSKAFS |
| NOV54d | DVPALLKLTCSKEHAIISVSVAIGVCYAFSCLVCIVVSYVYIFSAVLRISQRQRQSKAFS |
| NOV54e | DVPALLKLTCSKEHAIISVSGAIGVCYAFSCLVCIVVSYVYIFSAVLRISQRQRQSKAFS |
| NOV54f | DVPALLKLTCSKEHAIISVSVAIGVCYAFSCLVCIVVSYVYIFSAVLRISQRQRQSKAFS |
| NOV54g | DVPALLKLTCSKEHAIISVSVAIGVCYAFSCLVCIVVSYVYIFSAVLRISQRQRQSKAFS |
| NOV54h | DVPALLKLTCSKEHAIISVSVAIGVCYAFSCLVCIVVSYVYIFSAVLRISQRQRQSKAFS |
| NOV54i | DVPALLKLTCSKEHAIISVSVAIGVCYAFSCLVCIVVSYVYIFSAVLRISQRQRQSKAFS |
| NOV54j | ENWVLLRLHA-------------------------------------------------- |
| NOV54k | DVPALLKLTCSKEHAIISVS---------------------------------------- |
| NOV54l | DVPALLKLTCSKEHAIISVSVAIGVCYAFSCLVCIVVSYVYIFSAVLRISQRQRQSKAFS |
| NOV54a | NCVPHLIVVTVFLVTGAVAYLKPGSDAPSILDLLVSVFYSVAPPTLNPVIYCLKNKDIKS |
| NOV54b | NCVPHLIVVTVFLVTGAVAYLKPGSDAPSILDLLVSVFYSVAPPTLNPVIYCLKNKDIKS |
| NOV54c | NCVPHLIVVTVFLVTGAVAYLKPGSDTPSILDLLVSVFYSVAPPTLNPVIYCLKNKDIKS |
| NOV54d | NCVPHLIVVTVFLVTGAVAYLKPGSDAPSILDLLVSVFYSVAPPTLNPVIYCLKNKDIKS |
| NOV54e | NCVPHLIVVTVFLVTGAVAYLKPGSDAPSILDLLVSVFYSVAPPTLNPVIYCLKNKDIKS |
| NOV54f | NCVPHLIVVTVFLVTGAVAYLKPGSDAPSILDLLVSVFYSVAPPTLNPVIYCLKNKDIKS |
| NOV54g | NCVPHLIVVTVFLVTGAVAYLKPGSDAPSILDLLVSVFYSVAPPTLNPVIYCLKNKDIKS |
| NOV54h | NCVPHLIVVTVFLVTGAVAYLKPGSDAPSILDLLVSVFYSVAPPTLNPVIYCLKNKDIKS |
| NOV54i | NCVPHLIVVTVFLVTGAVAYLKPGSDAPSILDLLVSVFYSVAPPTLNPVIYCLKNKDIKS |
| NOV54j | ------------------------------------------------------------ |
| NOV54k | ------------------------------------------------------------ |
| NOV54l | NCVPHLIVVTVFLVTGAVAYLKPGSDAPSILDLLVSVFYSVAPPTLNPVIYCLKNKDIKS |
| NOV54a | ALSKVLWNVRSSGVMKDD-- |
| NOV54b | ALSKVLWNVRSSGVMKDD-- |
| NOV54c | ALSKVLWNVRSSGVMKDDLE |
| NOV54d | ALSKVLWNVRSSGVMKDDLE |
| NOV54e | ALSKVLWNVRSSGVMKDDLE |
| NOV54f | ALSKVLWNVRSSGVMKDDLE |
| NOV54g | ALSKVLWNVRSSGVMKDDLE |
| NOV54h | ALSKVLWNVRSSGVMKDD-- |
| NOV54i | ALSKVLWNVRSSGVMKDD-- |
| NOV54j | -------------------- |
| NOV54k | -------------------- |
| NOV54l | ALSKVLWNVRSSGVMKDD-- |
| NOV54a | (SEQ ID NO: 832) |
| NOV54b | (SEQ ID NO: 834) |
| NOV54c | (SEQ ID NO: 836) |
| NOV54d | (SEQ ID NO: 838) |
| NOV54e | (SEQ ID NO: 840) |
| NOV54f | (SEQ ID NO: 842) |
| NOV54g | (SEQ ID NO: 844) |
| NOV54h | (SEQ ID NO: 846) |
| NOV54i | (SEQ ID NO: 848) |
| NOV54j | (SEQ ID NO: 850) |
| NOV54k | (SEQ ID NO: 852) |
| NOV54l | (SEQ ID NO: 854) |

Further analysis of the NOV54a protein yielded the following properties shown in Table 54C.

TABLE 54C
Protein Sequence Properties NOV54a

| | |
|---|---|
| SignalP analysis: | Cleavage site between residues 70 and 71 |
| PSORT II analysis: | |

| | |
|---|---|
| PSG: | a new signal peptide prediction method |
| | N-region: length 6; pos.chg 1; neg.chg 1 |
| | H-region: length 9; peak value 7.79 |
| | PSG score: 3.39 |
| GvH: | von Heijne's method for signal seq. recognition |
| | GvH score (threshold: −2.1): −2.72 |
| | possible cleavage site: between 59 and 60 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 4 | | |
| | INTEGRAL | Likelihood = −9.66 | Transmembrane 44-60 |
| | INTEGRAL | Likelihood = −9.71 | Transmembrane 152-168 |
| | INTEGRAL | Likelihood = −5.79 | Transmembrane 193-209 |
| | INTEGRAL | Likelihood = 0.16 | Transmembrane 221-237 |

| | |
|---|---|
| PERIPHERAL | Likelihood = 1.11 (at 10) |

| | ALOM score: −9.71 (number of TMSs: 4) |
|---|---|
| MTOP: | Prediction of membrane topology (Hartmann et al.) |
| | Center position for calculation: 51 |
| | Charge difference: 0.0 C(−1.0) − N(−1.0) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 6.56 |
| Hyd Moment(95): | 1.99 | G content: | 0 |
| D/E content: | 2 | S/T content: | 1 |
| Score: −5.34 | | | |

Gavel: prediction of cleavage sites for mitochondrial preseq

R-2 motif at 26 LRH|LS

NUCDISC: discrimination of nuclear localization signals

pat4: none
pat7: none
bipartite: none
content of basic residues: 7.1%
NLS Score: −0.47

KDEL: ER retention motif in the C-terminus: none
ER Membrane Retention Signals:

KKXX-like motif in the C-terminus: VMKD

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

66.7%: endoplasmic reticulum
33.3%: mitochondrial

>> prediction for CG56146-02 is end (k = 9)

A search of the NOV54a protein against the Geneseq database, a proprietary database that contains sequences published in patents and patent publication, yielded several homologous proteins shown in Table 54D.

TABLE 54D
Geneseq Results for NOV54a

| | | NOV54a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU11100 | Human novel G protein-coupled | 1 . . . 269 | 268/269 (99%) | e−152 |
| | receptor, NOV7 - Homo sapiens, | 47 . . . 315 | 268/269 (99%) | |
| | 315 aa. [WO200177177-A2, 18- | | | |
| | OCT-2001] | | | |
| AAU85251 | G-coupled olfactory receptor #112 - | 1 . . . 269 | 268/269 (99%) | e−152 |
| | Homo sapiens, 315 aa. | 47 . . . 315 | 268/269 (99%) | |
| | [WO200198526-A2, 27-DEC- | | | |
| | 2001] | | | |
| AAU95586 | Human olfactory and pheromone | 1 . . . 269 | 268/269 (99%) | e−152 |
| | G protein-coupled receptor #73 - | 47 . . . 315 | 268/269 (99%) | |
| | Homo sapiens, 315 aa. | | | |
| | [WO200224726-A2, 28-MAR- | | | |
| | 2002] | | | |
| AAU24631 | Human olfactory receptor | 1 . . . 269 | 268/269 (99%) | e−152 |
| | AOLFR125 - Homo sapiens, 315 | 47 . . . 315 | 268/269 (99%) | |
| | aa. [WO200168805-A2, 20-SEP- | | | |
| | 2001] | | | |
| ABP95851 | Human GPCR polypeptide SEQ | 1 . . . 267 | 266/267 (99%) | e−151 |
| | ID NO 512 - Homo sapiens, 314 | 47 . . . 313 | 266/267 (99%) | |
| | aa. [WO200216548-A2, 28-FEB- | | | |
| | 2002] | | | |

In a BLAST search of public sequence databases, the NOV54a protein was found to have homology to the proteins shown in the BLASTP data in Table 54E.

TABLE 54E
Public BLASTP Results for NOV54a

| | | NOV54a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8NGZ2 | Seven transmembrane helix | 1 . . . 269 | 268/269 (99%) | e−152 |
| | receptor - Homo sapiens | 47 . . . 315 | 268/269 (99%) | |
| | (Human), 315 aa. | | | |
| Q96R53 | Olfactory receptor - Homo | 20 . . . 236 | 216/217 (99%) | e−120 |
| | sapiens (Human), 217 aa | 1 . . . 217 | 216/217 (99%) | |
| | (fragment). | | | |
| CAD35484 | Sequence 23 from Patent | 3 . . . 257 | 134/255 (52%) | 6e−77 |
| | WO0208289 - Homo sapiens | 49 . . . 303 | 181/255 (70%) | |
| | (Human), 314 aa. | | | |
| Q8NHC5 | Seven transmembrane helix | 3 . . . 257 | 138/259 (53%) | 2e−76 |
| | receptor - Homo sapiens | 49 . . . 302 | 192/259 (73%) | |
| | (Human), 309 aa. | | | |
| CAD35483 | Sequence 21 from Patent | 3 . . . 257 | 134/255 (52%) | 3e−76 |
| | WO0208289 - Homo sapiens | 49 . . . 303 | 180/255 (70%) | |
| | (Human), 314 aa. | | | |

PFam analysis indicates that the NOV54a protein contains the domains shown in the Table 54F.

TABLE 54F
Domain Analysis of NOV54a

| | | Identities/ | |
|---|---|---|---|
| | | Similarities | |
| Pfam | NOV54a | for the Matched | Expect |
| Domain | Match Region | Region | Value |
| 7tm_1 | 1 . . . 242 | 53/277 (19%) | 8.5e−07 |
| | | 162/277 (58%) | |

### Example 55

The NOV55 clone was analyzed, and the nucleotide and encoded polypeptide sequences are shown in Table 55A.

TABLE 55A
NOV55 Sequence Analysis

| | | |
|---|---|---|
| NOV55a, CG56258-04 | SEQ ID NO: 855 | 2828 bp |

| | | |
|---|---|---|
| DNA Sequence | ORF Start: ATG at 63 | ORF Stop: TAA at 2826 |

GTCTCTGGCCTATCAGGAGGACAACTGGTGCTGCAATAGAAGCCAGTGGCTAAGTCTCGTGTATGGCG
TGGTTAAGGTTGCAGCCTCTCACCTCTGCCTTCCTCCATTTTGGGCTGGTTACCTTTGTGCTCTTCCT
GAATGGTCTTCGAGCAGAGGCTGGTGGCTCAGGGGACGTGCCAAGCACAGGGCAGAACAATGAGTCCT
GTTCAGGGTCATCGGACTGCAAGGAGGGTGTCATCCTGCCAATCTGGTACCCGGAGAACCCTTCCCTT
GGGGACAAGATTGCCAGGGTCATTGTCTATTTTGTGGCCCTGATATACATGTTCCTTGGGGTGTCCAT
CATTGCTGACCGCTTCATGGCATCTATTGAAGTCATCACCTCTCAAGAGAGGGAGGTGACAATTAAGA
AACCCAATGGAGAAACCAGCACAACCACTATTCGGGTCTGGAATGAAACTGTCTCCAACCTGACCCTT
ATGGCCCTGGGTTCCTCTGCTCCTGAGATACTCCTCTCTTTAATTGAGGTGTGTGGTCATGGGTTCAT
TGCTGGTGATCTGGGACCTTCTACCATTGTAGGGAGTGCAGCCTTCAACATGTTCATCATCATTGGCA
TCTGTGTCTACGTGATCCCAGACGGAGAGACTCGCAAGATCAAACATCTACGAGTCTTCTTCATCACC
GCTGCTTGGAGTATCTTTGCCTACATCTGGCTCTATATGATTCTGGCAGTCTTCTCCCCTGGTGTGGT
CCAGGTTTGGGAAGGCCTCCTCACTCTCTTCTTCTTTCCAGTGTGTGTCCTTCTGGCCTGGGTGGCAG
ATAAACGACTGCTCTTCTACAAATACATGCACAAAAAGTACCGCACAGACAAACACCGAGGAATTATC
ATAGAGACAGAGGGTGACCACCCTAAGGGCATTGAGATGGATGGGAAAATGATGAATTCCCATTTTCT
GATGGGAACCTGGTGCCCCTGGAAGGGAAGGAAGTGGATGAGTCCCGCAGAGAGATGATCCGGAATTC
TCAAGGATCTGAAGCAAAAACACCCAGAGAAGGACTTAGATCAGCTGGTGGAGATGGCCAATTACTAT
GCTCTTTCCCACCAACAGAAGAGCCGCGCCTTCTACCGTATCCAAGCCACTCGTATGATGACTGGTGC
AGGCAATATCCTGAAGAAACATGCAGCAGAACAAGCCAAGAAGGCCTCCAGCATGAGCGAGGTGCACA
CCGATGAGCCTGAGGACTTTATTTCCAAGGTCTTCTTTGACCCATGTTCTTACCAGTGCCTGGAGAAC
TGTGGGGCTGTACTCCTGACAGTGGTGAGGAAAGGGGGAGACATGTCAAAGACCATGTATGTGGACTA
CAAAACAGAGGATGGTTCTGCCAATGCAGGGGCTGACTATGAGTTCACAGAGGGCACGGTGGTTCTGA
AGCCAGGAGAGACCCAGAAGGAGTTCTCCGTGGGCATAATTGATGACGACATTTTTGAGGAGGATGAA
CACTTCTTTGTAAGGTTGAGCAATGTCCGCATAGAGGAGGAGCAGCCAGAGGAGGGGATGCCTCCAGC
AATATTCAACAGTCTTCCCTTGCCTCGGGCTGTCCTAGCCTCCCCTTGTGTGGCCACAGTTACCATCT
TGGATGATGACCATGCAGGCATCTTCACTTTTGAATGTGATACTATTCATGTCAGTGAGAGTATTGGT
GTTATGGAGGTCAAGGTTCTGCGGACATCAGGTGCCCGGGGTACAGTCATCGTCCCCTTTAGGACAGT
AGAAGGGACAGCCAAGGGTGGCGGTGAGGACTTTGAAGACACATATGGGGAGTTGGAATTCAAGAATG
ATGAAACTGTGAAAACCATAAGGGTTAAAATAGTAGATGAGGAGGAATACGAAAGGCAAGAGAATTTC
TTCATTGCCCTTGGTGAACCGAAATGGATGGAACGTGGAATATCAGATGTGACAGACAGGAAGCTGAC
TATGGAAGAAGAGGAGGCCAAGAGGATAGCAGAGATGGGAAAGCCAGTATTGGGTGAACACCCCAAAC
TAGAACTCATCATTGAAGAGTCCTATGAGTTCAAGACTACGGTGGACAAACTGATCAAGAAGACAAAC
CTGGCCTTGGTTGTGGGGACCCATTCCTGGAGGGACCAGTTCATGGAGGCCATCACCGTCAGTGCAGC
AGGGGATGAGGATGAGGATGAATCCGGGGAGGAGAGGCTGCCCTCCTGCTTTGACTACGTCATGCACT
TCCTGACTGTCTTCTGGAAGGTGCTGTTTGCCTGTGTGCCCCCCACAGAGTACTGCCACGGCTGGGCC
TGCTTCGCCGTCTCCATCCTCATCATTGGCATGCTCACCGCCATCATTGGGGACCTGGCCTCGCACTT
CGGCTGCACCATTGGTCTCAAAGATTCAGTCACAGCTGTTGTTTTCGTGGCATTTGGCACCTCTGTCC
CAGATACGTTTGCCAGCAAAGCTGCTGCCCTCCAGGATGTATATGCAGACGCCTCCATTGGCAACGTG
ACGGGCAGCAACGCCGTCAATGTCTTCCTGGGCATCGGCCTGGCCTGGTCCGTGGCCGCCATCTACTG
GGCTCTGCAGGGACAGGAGTTCCACGTGTCGGCCGGCACACTGGCCTTCTCCGTCACCCTCTTCACCA
TCTTTGCATTTGTCTGCATCAGCGTGCTCTTGTACCGAAGGCGGCCGCACCTGGGAGGGGAGCTTGGT
GGCCCCCGTGGCTGCAAGCTCGCCACAACATGGCTCTTTGTGAGCCTGTGGCTCCTCTACATACTCTT
TGCCACACTAGAGGCCTATTGCTACATCAAGGGGTTCTAA

NOV55a, CG56258-04

| | | | |
|---|---|---|---|
| Protein Sequence | SEQ ID NO: 856 | 921 aa | MW at 1024 13.7kD |

MAWLRLQPLTSAFLHFGLVTFVLFLNGLRAEAGGSGDVPSTGQNNESCSGSSDCKEGVILPIWYPENP
SLGDKIARVIVYFVALIYMFLGVSIIADRFMASIEVITSQEREVTIKKPNGETSTTTIRVWNETVSNL
TLMALGSSAPEILLSLIEVCGHGFIAGDLGPSTIVGSAAFNMFIIIGICVYVIPDGETRKIKHLRVFF
ITAAWSIFAYIWLYMILAVFSPGVVQVWEGLLTLFFFPVCVLLAWVADKRLLFYKYMHKKYRTDKHRG
IIIETEGDHPKGIEMDGKMMNSHFLDGNLVPLEGKEVDESRREMIRILKDLKQKHPEKDLDQLVEMAN
YYALSHQQKSRAFYRIQATRMMTGAGNILKKHAAEQAKKASSMSEVHTDEPEDFISKVFFDPCSYQCL
ENCGAVLLTVVRKGGDMSKTMYVDYKTEDGSANAGADYEFTEGTVVLKPGETQKEFSVGIIDDDIFEE
DEHFFVRLSNVRIEEEQPEEGMPPAIFNSLPLPRAVLASPCVATVTILDDDHAGIFTFECDTIHVSES
IGVMEVKVLRTSGARGTVIVPFRTVEGTAKGGGEDFEDTYGELEFKNDETVKTIRVKIVDEEEYERQE
NFFIALGEPKWMERGISDVTDRKLTMEEEEAKRIAEMGKPVLGEHPKLEVIIEESYEFKTTVDKLIKK
TNLALVVGTHSWRDQFMEAITVSAAGDEDEDESGEERLPSCFDYVMHFLTVFWKVLFACVPPTEYCHG
WACFAVSILIIGMLTAIIGDLASHFGCTIGLKDSVTAVVFVAFGTSVPDTFASKAAALQDVYADASIG
NVTGSNAVNVFLGIGLAWSVAAIYWALQGQEFHVSAGTLAFSVTLFTIFAFVCISVLLYRRRPHLGGE
LGGPRGCKLATTWLFVSLWLLYILFATLEAYCYIKGF

| | | |
|---|---|---|
| NOV55b, CG56258-02 | SEQ ID NO: 857 | 2840 bp |

| | | |
|---|---|---|
| DNA Sequence | ORF Start: ATG at 63 | ORF Stop: TAA at 2838 |

GTCTCTGGCCTATCAGGAGGACAACTGGTGCTGCAATAGAAGCCAGTGGCTAAGTCTCGTGTATGGCG
TGGTTAAGGTTGCAGCCTCTCACCTCTGCCTTCCTCCATTTTGGGCTGGTTACCTTTGTGCTCTTCCT
GAATGGTCTTCGAGCAGAGGCTGGTGGCTCAGGGGACGTGCCAAGCACAGGGCAGAACAATGAGTCCT
GTTCAGGGTCATCGGACTGCAAGGAGGGTGTCATCCTGCCAATCTGGTACCCGGAGAACCCTTCCCTT
GGGGACAAGATTGCCAGGGTCATTGTCTATTTTGTGGCCCTGATATACATGTTCCTTGGGGTGTCCAT
CATTGCTGACCGCTTCATGGCATCTATTGAAGTCATCACCTCTCAAGAGAGGGAGGTGACAATTAAGA
AACCCAATGGAGAAACCAGCACAACCACTATTCGGGTCTGGAATGAAACTGTCTCCAACCTGACCCTT
ATGGCCCTGGGTTCCTCTGCTCCTGAGATACTCCTCTCTTTAATTGAGGTGTGTGGTCATGGGTTCAT
TGCTGGTGATCTGGGACCTTCTACCATTGTAGGGAGTGCAGCCTTCAACATGTTCATCATCATTGGCA
TCTGTGTCTACGTGATCCCAGACGGAGAGACTCGCAAGATCAAGCATCTACGAGTCTTCTTCATCACC
GCTGCTTGGAGTATCTTTGCCTACATCTGGCTCTATATGATTCTGGCAGTCTTCTCCCCTGGTGTGGT
CCAGGTTTGGGAAGGCCTCACTGCTCTCTTCTTCTTTCCAGTGTGTGTCCTTCTGGCCTGGGTGGCAG
ATAAACGACTGCTCTTCTACAAATACATGCACAAAAAGTACCGCACAGACAAACACCGAGGAATTATC
ATAGAGACAGAGGGTGACCACCCTAAGGGCATTGAGATGGGAAAATGGGTGATGAATTCCCATTTTCT
AGATGGGAACCTGGTGCCCCTGGAGGGAAGGGAAGTGGATGAGTCCCGCAGAGAGATGATCCGGATTC
TCAAGGATCTGAAGCAAAAACACCCAGAGAAGGACTTAGATCAGCTGGTGGAGATGGCCAATTACTAT
GCTCTTTCCCACCAACAGAAGAGCCGTGCCTTCTACCGTATCCAAGCCACTCGTATGATGACTGGTGC
AGGCAATATCCTGAAGAAACATGCAGCAGAACAAGCCAAGAAGGCCTCCAGCATGAGCGAGGTGCACA
CCGATGAGCCTGAGGACTTTATTTCCAAGGTCTTCTTTGACCCATGTTCTTACCAGTGCCTGGAGAAC
TGTGGGGCTGTACTCCTGACAGTGGTGAGGAAAGGGGGAGACATGTCAAAGACCATGTATGTGGACTA
CAAAACAGAGGATGGTTCTGCCAATGCAGGGGCTGACTATGAGTTCACAGAGGGCACGGTGGTTCTGA
AGCCAGGAGAGACCCAGAAGGAGTTCTCCGTGGGCATAATTGATGACGACATTTTTGAGGAGGATGAA
CACTTCTTTGTAAGGTTGAGCAATGTCCGCATAGAGGAGGAGCAGCCAGAGGAGGGGATGCCTCCAGC
AATATTCAACAGTCTTCCCTTGCCTCGGGCTGTCCTAGCCTCCCCTTGTGTGGCCACAGTTACCATCT
TGGATGATGACCATGCAGGCATCTTCACTTTTGAATGTGATACTATTCATGTCAGTGAGAGTATTGGT
GTTATGGAGGTCAAGGTTCTGCGGACATCAGGTGCCCGGGGTACAGTCATCGTCCCCTTTAGGACAGT
AGAAGGGACAGCCAAGGGTGGCGGTGAGGACTTTGAAGACACATATGGGGAGTTGGAATTCAAGAATG
ATGAAACTGTCAAAACAATTCACATCAAGGTAATTGATGATGAGGCATATGAGAAAAACAAGAATTAC
TTCATTGAGATGATGGGCCCCCGCATGGTGGATATGAGTTTTCAGAAAGCGCTCCTGTTATCTCCAGA
CAGGAAGCTGACTATGGAAGAAGAGGAGGCCAAGAGGATAGCAGAGATGGGAAAGCCAGTATTGGGTG
AACACCCCAAACTAGAAGTCATCATTGAAGAGTCCTATGAGTTCAAGACTACGGTGGACAAACTGATC
AAGAAGACAAACCTGGCCTTGGTTGTGGGGACCCATTCCTGGAGGGACCAGTTCATGGAGGCCATCAC
CGTCAGTGCAGCAGGGGATGAGGATGAGGATGAATCCGGGGAGGAGAGGCTGCCCTCCTGCTTTGACT
ACGTCATGCACTTCCTGACTGTCTTCTGGAAGGTGCTGTTTGCCTGTGTGCCCCCCACAGAGTACTGC
CACGGCTGGGCCTGCTTCGCCGTCTCCATCCTCATCATTGGCATGCTCACCGCCATCATTGGGGACCT
GGCCTCGCACTTCGGCTGCACCATTGGTCTCAAAGATTCAGTCACAGCTGTTGTTTTCGTGGCATTTG
GCACCTCTGTCCCAGATACGTTTGCCAGCAAAGCTGCTGCCCTCCAGGATGTATATGCAGACGCCTCC
ATTGGCAACGTGACGGGCAGCAACGCCGTCAATGTCTTCCTGGGCATCGGCCTGGCCTGGTCCGTGGC
CGCCATCTACTGGGCTCTGCAGGGACAGGAGTTCCACGTGTCGGCCGGCACACTGGCCTTCTCCGTCA
CCCTCTTCACCATCTTTGCATTTGTCTGCATCAGCGTGCTCTTGTACCGAAGGCGGCCGCACCTGGGA
GGGGAGCTTGGTGGCCCCCGTGGCTGCAAGCTCGCCACAACATGGCTCTTTGTGAGCCTGTGGCTCCT
CTACATACTCTTTGCCACACTAGAGGCCTATTGCTACATCAAGGGGTTCTAA

NOV55b, CG56258-02

| | | | |
|---|---|---|---|
| Protein Sequence | SEQ ID NO: 858 | 925 aa | MW at 102802.3kD |

MAWLRLQPLTSAFLHFGLVTFVLFLNGLRAEAGGSGDVPSTGQNNESCSGSSDCKEGVILPIWYPENP
SLGDKIARVIVYFVALIYMFLGVSIIADRFMASIEVITSQEREVTIKKPNGETSTTTIRVWNETVSNL
TLMALGSSAPEILLSLIEVCGHGFIAGDLGPSTIVGSAAFNMFIIIGICVYVIPDGETRKIKHLRVFF
ITAAWSIFAYIWLYMILAVFSPGVVQVWEGLLTLFFFPVCVLLAWVADKRLLFYKYMHKKYRTDKHRG
IIIETEGDHPKGIEMDGKMMNSHFLDGNLVPLEGKEVDESRREMIRILKDLKQKHPEKDLDQLVEMAN
YYALSHQQKSRAFYRIQATRMMTGAGNILKKHAAEQAKKASSMSEVHTDEPEDFISKVFFDPCSYQCL
ENCGAVLLTVVRKGGDMSKTMYVDYKTEDGSANAGADYEFTEGTVVLKPGETQKEFSVGIIDDDIFEE
DEHFFVRLSNVRIEEEQPEEGMPPAIFNSLPLPRAVLASPCVATVTILDDDHAGIFTFECDTIHVSES
IGVMEVKVLRTSGARGTVIVPFRTVEGTAKGGGEDFEDTYGELEFKNDETVKTIHIKVIDDEAYEKNK
NYFIEMMGPRMVDMSFQKALLLSPDRKLTMEEEEAKRIAEMGKPVLGEHPKLEVIIEESYEFKTTVDK
LIKKTNLALVVGTHSWRDQFMEAITVSAAGDEDEDESGEERLPSCFDYVMHFLTVFWKVLFACVPPTE
YCHGWACFAVSILIIGMLTAIIGDLASHFGCTIGLKDSVTAVVFVAFGTSVPDTFASKAAALQDVYAD
ASIGNVTGSNAVNVFLGIGLAWSVAAIYWALQGQEFHVSAGTLAFSVTLFTIFAFVCISVLLYRRRPH
LGGELGGPRGCKLATTWLFVSLWLLYILFATLEAYCYIKGF

| | | |
|---|---|---|
| NOV55c, 258076220 | SEQ ID NO: 859 | 2778 bp |

| | | |
|---|---|---|
| DNA Sequence | ORF Start: at 1 | ORF Stop: end of sequence |

GGATCCACCATGGCGTGGTTAAGGTTGCAGCCTCTCACCTCTGCCTTCCTCCATTTTGGGCTGGTTAC
CTTTGTGCTCTTCCTGAATGGTCTTCGAGCAGAGGCTGGTGGCTCAGGGGACGTGCCAAGCACAGGGC
AGAACAATGAGTCCTGTTCAGGGTCATCGGACTGCAAGGAGGGTGTCATCCTGCCAATCTGGTACCCG
GAGAACCCTTCCCTTGGGGACAAGATTGCCAGGGTCATTGTCTATTTTGTGGCCCTGATATACATGTT
CCTTGGGGTGTCCATCATTGCTGACCGCTTCATGGCATCTATTGAAGTCATCACCTCTCAAGAGAGGG
AGGTGACAATTAAGAAACCCAATGGAGAAACCAGCACAACCACTATTCGGGTCTGGAATGAAACTGTC
TCCAACCTGACCCTTATGGCCCTGGGTTCCTCTGCTCCTGAGATACTCCTCTCTTTAATTGAGGTGTG
TGGTCATGGGTTCATTGCTGGTGATCTGGGACCTTCTACCATTGTAGGGAGTGCAGCCTTCAACATGT
TCATCATCATTGGCATCTGTGTCTACGTGATCCCAGACGGAGAGACTCGCAAGATCAAGCATCTACGA
GTCTTCTTCATCACCGCTGCTTGGAGTATCTTTGCCTACATCTGGCTCTATATGATTCTGGCAGTCTT
CTCCCCTGGTGTGGTCCAGGTTTGGGAAGGCCTCCTCACTCTCTTCTTCTTTCCAGTGTGTGTCCTTC
TGGCCTGGGTGGCAGATAAACGACTGCTCTTCTACAAATACATGCACAAAAAGTACCGCACAGACAAA
CACCGAGGAATTATCATAGAGACAGAGGGTGACCACCCTAAGGGCATTGAGATGGATGGGAAAATGAT
GAATTCCCATTTTCTAGATGGGAACCTGGTGCCCCTGGAAGGGAAGGAAGTGGATGAGTCCCGCAGAG
AGATGATCCGGATTCTCAAGGATCTGAAGCAAAAACACCCAGAGAAGGACTTAGATCAGCTGGTGGAG
ATGGCCAATTACTATGCTCTTTCCCACCAACAGAAGAGCCGCGCCTTCTACCGTATCCAAGCCACTCG
TATGATGACTGGTGCAGGCAATATCCTGAAGAAACATGCAGCAGAACAAGCCAAGAAGGCCTCCAGCA
TGAGCGAGGTGCACACCGATGAGCCTGAGGACTTTATTTCCAAGGTCTTCTTTGACCCATGTTCTTAC
CAGTGCCTGGAGAACTGTGGGGCTGTACTCCTGACAGTGGTGAGGAAAGGGGGAGACATGTCAAAGAC
CATGTATGTGGACTACAAAACAGAGGATGGTTCTGCCAATGCAGGGGCTGACTATGAGTTCACAGAGG
GCACGGTGGTTCTGAAGCCAGGAGAGACCCAGAAGGAGTTCTCCGTGGGCATAATTGATGACGACATT
TTTGAGGAGGATGAACACTTCTTTGTAAGGTTGAGCAATGTCCGCATAGAGGAGGAGCAGCCAGAGGA
GGGGATGCCTCCAGCAATATTCAACAGTCTTCCCTTGCCTCGGGCTGTCCTAGCCTCCCCTTGTGTGG
CCACAGTTACCATCTTGGATGATGACCATGCAGGCATCTTCACTTTTGAATGTGATACTATTCATGTC
AGTGAGAGTATTGGTGTTATGGAGGTCAAGGTTCTGCGGACATCAGGTGCCCGGGGTACAGTCATCGT
CCCCTTTAGGACAGTAGAAGGGACAGCCAAGGGTGGCGGTGAGGACTTTGAAGACACATATGGGGAGT
TGGAATTCAAGAATGATGAAACTGTGAAAACCATAAGGGTTAAAATAGTAGATGAGGAGGAATACGAA
AGGCAAGAGAATTTCTTCATTGCCCTTGGTGAACCGAAATGGATGGAACGTGGAATATCAGATGTGAC
AGACAGGAAGCTGACTATGGAAGAAGAGGAGGCCAAGAGGATAGCAGAGATGGGAAAGCCAGTATTGG
GTGAACACCCCAAACTAGAAGTCATCATTGAAGAGTCCTATGAGTTCAAGACTACGGTGGACAAACTG
ATCAAGAAGACAAACCTGGCCTTGGTTGTGGGGACCCATTCCTGGAGGGACCAGTTCATGGAGGCCAT
CACCGTCAGTGCAGCAGGGGATGAGGATGAGGATGAATCCGGGGAGGAGAGGCTGCCCTCCTGCTTTG
ACTACGTCATGCACTTCCTGACTGTCTTCTGGAAGGTGCTGTTTGCCTGTGTGCCCCCCACAGAGTAC
TGCCACGGCTGGGCCTGCTTCGCCGTCTCCATCCTCATCATTGGCATGCTCACCGCCATCATTGGGGA
CCTGGCCTCGCACTTCGGCTGCACCATTGGTCTCAAAGATTCAGTCACAGCTGTTGTTTTCGTGGCAT
TTGGCACCTCTGTCCCAGATACGTTTGCCAGCAAAGCTGCTGCCCTCCAGGATGTATATGCAGACGCC
TCCATTGGCAACGTGACGGGCAGCAACGCCGTCAATGTCTTCCTGGGCATCGGCCTGGCCTGGTCCGT
GGCCGCCATCTACTGGGCTCTGCAGGGACAGGAGTTCCACGTGTCGGCCGGCACACTGGCCTTCTCCG
TCACCCTCTTCACCATCTTTGCATTTGTCTGCATCAGCGTGCTCTTGTACCGAAGGCGGCCGCACCTG
GGAGGGGAGCTTGGTGGCCCCCGTGGCTGCAAGCTCGCCACAACATGGCTCTTTGTGAGCCTGTGGCT
CCTCTACATACTCTTTGCCACACTAGAGGCCTATTGCTACATCAAGGGGTTCCTCGAG

NOV55c, 258076220

| | | | |
|---|---|---|---|
| Protein Sequence | SEQ ID NO: 860 | 926 aa | MW at 102901.2kD |

GSTMAWLRLQPLTSAFLHFGLVTFVLFLNGLRAEAGGSGDVPSTGQNNESCSGSSDCKEGVILPIWYP
ENPSLGDKIARVIVYFVALIYMFLGVSIIADRFMASIEVITSQEREVTIKKPNGETSTTTIRVWNETV
SNLTLMALGSSAPEILLSLIEVCGHGFIAGDLGPSTIVGSAAFNMFIIIGICVYVIPDGETRKIKHLR
VFFITAAWSIFAYIWLYMILAVFSPGVVQVWEGLLTLFFFPVCVLLAWVADKRLLFYKRMHKKYRTDK
HRGIIIETEGDHPKGIEMDGKMMNSHFLDGNLVPLEGKEVDESRREMIRILKDLKQKHPEKDLDQLVE
MANYYALSHQQKSRAFYRIQATRMMTGAGNILKKHAAEQAKKASSMSEVHTDEPEDFISKVFFDPCSY
QCLENCGAVLLTVVRKGGDMSKTMYVDYKTEDGSANAGADYEFTEGTVVLKPGETQKEFSVGIIDDDI
FEEDEHFFVRLSNVRIEEEQPEEGMPPAIFNSLPLPRAVLASPCVATVTILDDDHAGIFTFECDTIHV
SESIGVMEVKVLRTSGARGTVIVPFRTVEGTAKGGGEDFEDTYGELEFKNDETVKTIRVKIVDEEEYE
RQENFFIALGEPKWMERGISDVTDRKLTMEEEEAKRIAEMGKPVLGEHPKLEVIIEESYEFKTTVDKL
IKKTNLALVVGTHSWRDQFMEAITVSAAGDEDEDESGEERLPSCFDYVMHFLTVFWKVLFACVPPTEY
CHGWACFAVSILIIGMLTAIIGDLASHFGCTIGLKDSVTAVVFVAFGTSVPDTFASKAAALQDVYADA
SIGNVTGSNAVNVFLGIGLAWSVAAIYWALQGQEFHVSAGTLAFSVTLFTIFAFVCISVLLYRRRPHL
GGELGGPRGCKLATTWLFVSLWLLYILFATLEAYCYIKGFLE

| | | |
|---|---|---|
| NOV55d, 248057963 | SEQ ID NO: 861 | 2685 bp |

| | | |
|---|---|---|
| DNA Sequence | ORF Start: at 1 | ORF Stop: end of sequence |

GGATCCGAGGCTGGTGGCTCAGGGGACGTGCCAAGCACAGGGCAGAACAATGAGTCCTGTTCAGGGTC
ATCGGACTGCAAGGAGGGTGTCATCCTGCCAATCTGGTACCCGGAGAACCCTTCCCTTGGGGACAAGA
TTGCCAGGGTCATTGTCTATTTTGTGGCCCTGATATACATGTTCCTTGGGGTGTCCATCATTGCTGAC
CGCTTCATGGCATCTATTGAGTCATCACCTCTCAAGAGAGGGAGGTGACAATTAAGAAACCCAATTGG
AGAAACCAGCACAACCACTATTCGGGTCTGGAATGAAACTGTCTCCAACCTGACCCTTATGGCCCTGG
GTTCCTCTGCTCCTGAGATACTCCTCTCTTTAATTGAGGTGTGTGGTCATGGGTTCATTGCTGGTGAT
CTGGGACCTTCTACCATTGTAGGGAGTGCAGCCTTCAACATGTTCATCATCATTGGCATCTGTGTCTA
CGTGATCCCAGACGGAGAGACTCGCAAGATCAAACATCTACGAGTCTTCTTCATCACCGCTGCTTGGA
GTATCTTTGCCTACATCTGGCTCTATATGATTCTGGCAGTCTTCTCCCCTGGTGTGGTCCAGGTTTGG
GAAGGCCTCCTCACTCTCTTCTTCTTTCCAGTGTGTGTCCTTCTGGCCTGGGTGGCAGATAAACGACT
GCTCTTCTACAAATACATGCACAAAAAGTACCGCACAGACAAACACCGAGGAATTATCATAGAGACAG
AGGGTGACCACCCTAAGGGCATTGAGATGGATGGGAAAATGATGAATTCCCATTTTCTAGATGGGAAC
CTGGTGCCCCTGGAAGGGAAGGAAGTGGATGAGTCCCGCAGAGAGATGATCCGGATTCTCAAGGATCT
GAAGCAAAAACACCCAGAGAAGGACTTAGATCAGCTGGTGGAGATGGCCAATTACTATGCTCTTTCCC
ACCAACAGAAGAGCCGCGCCTTCTACCGTATCCAAGCCACTCGTATGATGACTGGTGCAGGCAATATC
CTGAAGAAACATGCAGCAGAACAAGCCAAGAAGGCCTCCAGCATGAGCGAGGTGCACACCGATGAGCC
TGAGGACTTTATTTCCAAGGTCTTCTTTGACCCATGTTCTTACCAGTGCCTGGAGAACTGTGGGGCTG
TACTCCTGACAGTGGTGAGGAAAGGGGGAGACATGTCAAAGACCATGTATGTGGACTACAAAACAGAG
GATGGTTCTGCCAATGCAGGGGCTGACTATGAGTTCACAGAGGGCACGGTGGTTCTGAAGCCAGGAGA
GACCCAGAAGGAGTTCTCCGTGGGCATAATTGATGACGACATTTTTGAGGAGGATGAACACTTCTTTG
TAAGGTTGAGCAATGTCCGCATAGAGGAGGAGCAGCCAGAGGAGGGGATGCCTCCAGCAATATTCAAC
AGTCTTCCCTTGCCTCGGGCTGTCCTAGCCTCCCCTTGTGTGGCCACAGTTACCATCTTGGATGATGA
CCATGCAGGCATCTTCACTTTTGAATGTGATACTATTCATGTCAGTGAGAGTATTGGTGTTATGGAGG
TCAAGGTTCTGCGGACATCAGGTGCCCGGGGTACAGTCATCGTCCCCTTTAGGACAGTAGAAGGGACA
GCCAAGGGTGGCGGTGAGGACTTTGAAGACACATATGGGGAGTTGGAATTCAAGAATGATGAAACTGT
GAAAACCATAAGGGTTAAAATAGTAGATGAGGAGGAATACGAAAGGCAAGAGAATTTCTTCATTGCCC
TTGGTGAACCGAAATGGATGGAACGTGGAATATCAGATGTGACAGACAGGAAGCTGACTATGGAAGAA
GAGGAGGCCAAGAGGATAGCAGAGATGGGAAAGCCAGTATTGGGTGAACACCCCAAACTAGAAGTCAT
CATTGAAGAGTCCTATGAGTTCAAGACTACGGTGGACAAACTGATCAAGAAGACAAACCTGGCCTTGG
TTGTGGGGACCCATTCCTGGAGGGACCAGTTCATGGAGGCCATCACCGTCAGTGCAGCAGGGGATGAG
GATGAGGATGAATCCGGGGAGGAGAGGCTGCCCTCCTGCTTTGACTACGTCATGCACTTCCTGACTGT
CTTCTGGAAGGTGCTGTTTGCCTGTGTGCCCCCCACAGAGTACTGCCACGGCTGGGCCTGCTTCGCCG
TCTCCATCCTCATCATTGGCATGCTCACCGCCATCATTGGGGACCTGGCCTCGCACTTCGGCTGCACC
ATTGGTCTCAAAGATTCAGTCACAGCTGTTGTTTTCGTGGCATTTGGCACCTCTGTCCCAGATACGTT
TGCCAGCAAAGCTGCTGCCCTCCAGGATGTATATGCAGACGCCTCCATTGGCAACGTGACGGGCAGCA
ACGCCGTCAATGTCTTCCTGGGCATCGGCCTGGCCTGGTCCGTGGCCGCCATCTACTGGGCTCTGCAG
GGACAGGAGTTCCACGTGTCGGCCGGCACACTGGCCTTCTCCGTCACCCTCTTCACCATCTTTGCATT
TGTCTGCATCAGCGTGCTCTTGTACCGAAGGCGGCCGCACCTGGGAGGGGAGCTTGGTGGCCCCCGTG
GCTGCAAGCTCGCCACAACATGGCTCTTTGTGAGCCTGTGGCTCCTCTACATACTCTTTGCCACACTA
GAGGCCTATTGCTACATCAAGGGGTTCCTCGAG

NOV55d, 248057963

| | | | |
|---|---|---|---|
| Protein Sequence | SEQ ID NO: 862 | 895 aa | MW at 99385.0kD |

GSEAGGSGDVPSTGQNNESCSGSSDCKEGVILPIWYPENPSLGDKIARVIVYFVALIYMFLGVSIIAD
RFMASIEVITSQEREVTIKKPNGETSTTTIRVWNETVSNLTLMALGSSAPEILLSLIEVCGHGFIAGD
LGPSTIVGSAAFNNFIIIGICVYVIPDGETRKIKHLRVFFITAAWSIFAYIWLYMILAVFSPGVVQVW
EGLLTLFFFPVCVLLAWVADKRLLFYKYMHKKYRTDKHRGIIIETEGDHPKGIEMDGKMMNSHFLDGN
LVPLEGKEVDESRREMIRILKDLKQKHPEKDLDQLVEMANYYALSHQQKSRAFYRIQATRNMTGAGNI
LKKHAAEQAKKASSMSEVNTDEPEDFISKVFFDPCSYQCLENCGAVLLTVVRKGGDMSKTMYVDYKTE
DGSANAGADYEFTEGTVVLKPGETQKEFSVGIIDDDIFEEDEHFFVRLSNVRIEEEQPEEGMPPAIFN
SLPLPRAVLASPCVATVTILDDDHAGIFTFECDTIHVSESIGVMEVKVLRTSGARGTVIVPFRTVEGT
AKGGGEDFEDTYGELEFKNDETVKTIRVKIVDEEEYERQENFFIALGEPKWMERGISDVTDRKLTMEE
EEAKRIAEMGKPVLGEHPKLEVIIEESYEFKTTVDKLIKKTNLALVVGTHSWRDQFMEAITVSAAGDE
DEDESGEERLPSCFDYVMHFLTVFWKVLFACVPPTEYCHGWACFAVSILIIGMLTAIIGDLASHFGCT
IGLKDSVTAVVFVAFGTSVPDTFASKAAALQDVYADASIGNVTGSNAVNVFLGIGLAWSVAAIYWALQ
GQEFHVSAGTLAFSVTLFTIFAFVCISVLLYRRRPHLGGELGGPRGCKLATTWLFVSLWLLYILFATL
EAYCYIKGFLE

| | | |
|---|---|---|
| NOV55e, CG56258-01 | SEQ ID NO: 863 | 2813 bp |

| | | |
|---|---|---|
| DNA Sequence | ORF Start: ATG at 9 | ORF Stop: TAG at 2793 |

TCTCGTGTATGGCGTGGTTAAGGTTGCAGCCTCTCACCTCTGCCTTCCTCCATTTTGGGCTGGTTACC
TTTGTGCTCTTCCTGAATGGTCTTCGAGCAGAGGCTGGTGGCTCAGGGGACGTGCCAAGCACAGGGCA
GAACAATGAGTCCTGTTCAGGGTCATCGGACTGCAAGGAGGGTGTCATCCTGCCAATCTGGTACCCGG
AGAACCCTTCCCTTGGGGACAAGATTGCCAGGGTCATTGTCTATTTTGTGGCCCTGATATACATGTTC
CTTGGGGTGTCCATCATTGCTGACCGCTTCATGGCATCTATTGAAGTCATCACCTCTCAAGAGAGGGA
GGTGACAATTAAGAAACCCAATGGAGAAACCAGCACAACCACTATTCGGGTCTGGAATGAAACTGTCT
CCAACCTGACCCTTATGGCCCTGGGTTCCTCTGCTCCTGAGATACTCCTCTCTTTAATTGAGGTGTGT
GGTCATGGGTTCATTGCTGGTGATCTGGGACCTTCTACCATTGTAGGGAGTGCAGCCTTCAACATGTT
CATCATCATTGGCATCTGTGTCTACGTGATCCCAGACGGAGAGACTCGCAAGATCAAGCATCTACGAG
TCTTCTTCATCACCGCTGCTTGGAGTATCTTTGCCTACATCTGGCTCTATATGATTCTGGCAGTCTTC
CCCCCTGGTGTGGTCCAGGTTTGGGAAGGCCTCCTCACTCTCTTCTTCTTTCCAGTGTGTGTCCTTCT
GGCCTGGGTGGCAGATAAACGACTGCTCTTCTACAAATACATGCACAAAAAGTACCGCACAGACAAAC
ACCGAGGAATTATCATAGAGACAGAGGGTGACCACCCTAAGGGCATTGAGATGGATGGGAAAATGATG
AATTCCCATTTTCTAGATGGGAACCTGGTGCCCCTGGAAGGGAAGGAAGTGGATGAGTCCCGCAGAGA
GATGATCCGGATTCTCAAGGATCTGAAGCAAAAACACCCAGAGAAGGACTTAGATCAGCTGGTGGAGA
TGGCCAATTACTATGCTCTTTCCCACCAACAGAAGAGCCGTGCCTTCTACCGTATCCAAGCCACTCGT
ATGATGACTGGTGCAGGCAATATCCTGAAGAAACATGCAGCAGAACAAGCCAAGAAGGCCTCCAGCAT
GAGCGAGGTGCACACCGATGAGCCTGAGGACTTTATTTCCAAGGTCTTCTTTGACCCATGTTCTTACC
AGTGCCTGGAGAACTGTGGGGCTGTACTCCTGACAGTGGTGAGGAAAGGGGGAGACATGTCAAAGACC
ATGTATGTGGACTACAAAACAGAGGATGGTTCTGCCAATGCAGGGGCTGACTATGAGTTCACAGAGGG
CACGGTGGTTCTGAAGCCAGGAGAGACCCAGAAGGAGTTCTCCGTGGGCATAATTGATGACGACATTT
TTGAGGAGGATGAACACTTCTTTGTAAGGTTGAGCAATGTCCGCATAGAGGAGGAGCAGCCAGAGGAG
GGGATGCCTCCAGCAATATTCAACAGTCTTCCCTTGCCTCGGGCTGTCCTAGCCTCCCCTTGTGTGGC
CACAGTTACCATCTTGGATGATGACCATGCAGGCATCTTCACTTTTGAATGTGATACTATTCATGTCA
GTGAGAGTATTGGTGTTATGGAGGTCAAGGTTCTGCGGACATCAGGTGCCCGGGGTACAGTCATCGTC
CCCTTTAGGACAGTAGAAGGGACAGCCAAGGGTGGCGGTGAGGACTTTGAAGACACATATGGGGAGTT
GGAATTCAAGAATGATGAAACTGTGAAAACTCTTCAGGTGAAGATAGTTGATGACGAGGAATATGAGA
AAAAGGATAATTTCTTCATTGAGCTGGGCCAGCCCCAGTGGCTTAAGCGAGGGATTTCAGCTCTGCTA
CTCAATCAAGGGGATGGGGACAGGAAGCTAACAGCCGAGGAGGAGGAGGCTCGGAGGATAGCAGAGAT
GGGCAAGCCAGTTCTTGGGGAGAACTGCCGGCTGGAGGTCATCATCGAGGAGTCATATGATTTTAAGA
ACACGGTGGATAAACTCATCAAGAAAACGAACTTGGCCTTGGTAATTGGGACCCATTCATGGAGGGAG
CAGTTTTTAGAGGCAATTACGGTGAGCGCAGGGGACGAGGAGGAGGAGGAGGACGGGTCCCGGGAGGA
GCGGCTGCCGTCGTGCTTTGACTACGTGATGCACTTCCTGACGGTGTTCTGGAAGGTGCTCTTCGCCT
GTGTGCCCCCCACCGAGTACTGCCACGGCTGGGCCTGCTTTGGTGTCTCCATCCTGGTCATCGGCCTG
CTCACCGCCCTCATTGGGGACCTCGCCTCCCACTTCGGCTGCACCGTTGGCCTCAAGGACTCTGTCAA
TGCTGTTGTCTTCGTTGCCCTGGGCACCTCCATCCCTGACACGTTCGCCAGCAAGGTGGCGGCGCTGC
AGGACCAGTGCGCCGACGCGTCCATCGGCAACGTGACCGGCTCCAACGCGGTGAACGTGTTCCTTGGC
CTGGGCGTCGCCTGGTCTGTGGCCGCCGTGTACTGGGCGGTGCAGGGCCGCCCCTTCGAGGTGCGCAC
TGGCACGCTGGCCTTCTCCGTCACGCTCTTCACCGTCTTCGCCTTCGTGGGCATTGCCGTGCTGCTGT
ACCGGCGCCGGCCGCACATCGGCGGCGAGCTGGGCGGCCCGCGCGGACCCAAGCTCGCCACCACCGCG
CTCTTCCTGGGCCTCTGGCTCCTGTACATCCTCTTCGCCAGCCTGGAGGCGTACTGCCACATCCGGGG
CTTCTAGGGCCTCGCGCAGAGACTC

NOV55e, CG56258-01

| | | | |
|---|---|---|---|
| Protein Sequence | SEQ ID NO: 864 | 928 aa | MW at 102900.1kD |

MAWLRLQPLTSAFLHFGLVTFVLFLNGLRAEAGGSGDVPSTGQNNESCSGSSDCKEGVILPIWYPENP
SLGDKIARVIVYFVALIYMFLGVSIIADRFMASIEVITSQEREVTIKKPNGETSTTTIRVWNETVSNL
TLMALGSSAPEILLSLIEVCGHGFIAGDLGPSTIVGSAAFNNFIIIGICVYVIPDGETRKIKHLRVFF
ITAAWSIFAYIWLYMILAVFSPGVVQVWEGLLTLFFFPVCVLLAWVADKRLLFYKYMHKKYRTDKHRG
IIIETEGDHPKGIEMDGKMMNSHFLDGNLVPLEGKEVDESRREMIRILKDLKQKHPEKDLDQLVEMAN
YYALSHQQKSRAFYRIQATRMMTGAGNILKKHAAEQAKKASSMSEVHTDEPEDFISKVFFDPCSYQCL
ENCGAVLLTVVRKGGDMSKTMYVDYKTEDGSANAGADYEFTEGTVVLKPGETQKEFSVGIIDDDIFEE
DEHFFVRLSNVRIEEEQPEEGMPPAIFNSLPLPRAVLASPCVATVTILDDDHAGIFTFECDTIHVSES
IGVMEVKVLRTSGARGTVIVPFRTVEGTAKGGGEDFEDTYGELEFKNDETVKTLQVKIVDDEEYEKKD
NFFIELGQPQWLKRGISALLLNQGDGDRKLTAEEEEARRIAEMGKPVLGENCRLEVIIEESYDFKNTV
DKLIKKTNLALVIGTHSWREQFLEAITVSAGDEEEEEDGSREERLPSCFDYVMHFLTVFWKVLFACVP
PTEYCHGWACFGVSILVIGLLTALIGDLASHFGCTVGLKDSVNAVVFVALGTSIPDTFASKVAALQDQ
CADASIGNVTGSNAVNVFLGLGVAWSVAAVYWAVQGRPFEVRTGTLAFSVTLFTVFAFVGIAVLLYRR
RPHIGGELGGPRGPKLATTALFLGLWLLYILFASLEAYCHIRGF

| | | |
|---|---|---|
| NOV55f, CG56258-03 | SEQ ID NO: 865 | 2685 bp |

| | | |
|---|---|---|
| DNA Sequence | ORF Start: at 7 | ORF Stop: at 2680 |

GGATCCGAGGCTGGTGGCTCAGGGGACGTGCCAAGCACAGGGCAGAACAATGAGTCCTGTTCAGGGTC
ATCGGACTGCAAGGAGGGTGTCATCCTGCCAATCTGGTACCCGGAGAACCCTTCCCTTGGGGACAAGA
TTGCCAGGGTCATTGTCTATTTTGTGGCCCTGATATACATGTTCCTTGGGGTGTCCATCATTGCTGAC
CGCTTCATGGCATCTATTGAAGTCATCACCTCTCAAGAGAGGGAGGTGACAATTAAGAAACCCAATGG
AGAAACCAGCACAACCACTATTCGGGTCTGGAATGAAACTGTCTCCAACCTGACCCTTATGGCCCTGG
GTTCCTCTGCTCCTGAGATACTCCTCTCTTTAATTGAGGTGTGTGGTCATGGGTTCATTGCTGGTGAT
CTGGGACCTTCTACCATTGTAGGGAGTGCAGCCTTCAACATGTTCATCATCATTGGCATCTGTGTCTA
CGTGATCCCAGACGGAGAGACTCGCAAGATCAAACATCTACGAGTCTTCTTCATCACCGCTGCTTGGA
GTATCTTTGCCTACATCTGGCTCTATATGATTCTGGCAGTCTTCTCCCCTGGTGTGGTCCAGGTTTGG
GAAGGCCTCCTCACTCTCTTCTTCTTTCCAGTGTGTGTCCTTCTGGCCTGGGTGGCAGATAAACGACT
GCTCTTCTACAAATACATGCACAAAAAGTACCGCACAGACAAACACCGAGGAATTATCATAGAGACAG
AGGGTGACCACCCTAAGGGCATTGAGATGGATGGGAAAATGATGAATTCCCATTTTCTAGATGGGAAC
CTGGTGCCCCTGGAAGGGAAGGAAGTGGATGAGTCCCGCAGAGAGATGATCCGGATTCTCAAGGATCT
GAAGCAAAAACACCCAGAGAAGGACTTAGATCAGCTGGTGGAGATGGCCAATTACTATGCTCTTTCCC
ACCAACAGAAGAGCCGCGCCTTCTACCGTATCCAAGCCACTCGTATGATGACTGGTGCAGGCAATATC
CTGAAGAAACATGCAGCAGAACAAGCCAAGAAGGCCTCCAGCATGAGCGAGGTGCACACCGATGAGCC
TGAGGACTTTATTTCCAAGGTCTTCTTTGACCCATGTTCTTACCAGTGCCTGGAGAACTGTGGGGCTG
TACTCCTGACAGTGGTGAGGAAAGGGGGAGACATGTCAAAGACCATGTATGTGGACTACAAAACAGAG
GATGGTTCTGCCAATGCAGGGGCTGACTATGAGTTCACAGAGGGCACGGTGGTTCTGAAGCCAGGAGA
GACCCAGAAGGAGTTCTCCGTGGGCATAATTGATGACGACATTTTTGAGGAGGATGAACACTTCTTTG
TAAGGTTGAGCAATGTCCGCATAGAGGAGGAGCAGCCAGAGGAGGGGATGCCTCCAGCAATATTCAAC
AGTCTTCCCTTGCCTCGGGCTGTCCTAGCCTCCCCTTGTGTGGCCACAGTTACCATCTTGGATGATGA
CCATGCAGGCATCTTCACTTTTGAATGTGATACTATTCATGTCAGTGAGAGTATTGGTGTTATGGAGG
TCAAGGTTCTGCGGACATCAGGTGCCCGGGGTACAGTCATCGTCCCCTTTAGGACAGTAGAAGGGACA
GCCAAGGGTGGCGGTGAGGACTTTGAAGACACATATGGGGAGTTGGAATTCAAGAATGATGAAACTGT
GAAAACCATAAGGGTTAAAATAGTAGATGAGGAGGAATACGAAAGGCAAGAGAATTTCTTCATTGCCC
TTGGTGAACCGAAATGGATGGAACGTGGAATATCAGATGTGACAGACAGGAAGCTGACTATGGAAGAA
GAGGAGGCCAAGAGGATAGCAGAGATGGGAAAGCCAGTATTGGGTGAACACCCCAAACTAGAAGTCAT
CATTGAAGAGTCCTATGAGTTCAAGACTACGGTGGACAAACTGATCAAGAAGACAAACCTGGCCTTGG
TTGTGGGGACCCATTCCTGGAGGGACCAGTTCATGGAGGCCATCACCGTCAGTGCAGCAGGGGATGAG
GATGAGGATGAATCCGGGGAGGAGAGGCTGCCCTCCTGCTTTGACTACGTCATGCACTTCCTGACTGT
CTTCTGGAAGGTGCTGTTTGCCTGTGTGCCCCCCACAGAGTACTGCCACGGCTGGGCCTGCTTCGCCG
TCTCCATCCTCATCATTGGCATGCTCACCGCCATCATTGGGGACCTGGCCTCGCACTTCGGCTGCACC
ATTGGTCTCAAAGATTCAGTCACAGCTGTTGTTTTCGTGGCATTTGGCACCTCTGTCCCAGATACGTT
TGCCAGCAAAGCTGCTGCCCTCCAGGATGTATATGCAGACGCCTCCATTGGCAACGTGACGGGCAGCA
ACGCCGTCAATGTCTTCCTGGGCATCGGCCTGGCCTGGTCCGTGGCCGCCATCTACTGGGCTCTGCAG
GGACAGGAGTTCCACGTGTCGGCCGGCACACTGGCCTTCTCCGTCACCCTCTTCACCATCTTTGCATT
TGTCTGCATCAGCGTGCTCTTGTACCGAAGGCGGCCGCACCTGGGAGGGGAGCTTGGTGGCCCCCGTG
GCTGCAAGCTCGCCACAACATGGCTCTTTGTGAGCCTGTGGCTCCTCTACATACTCTTTGCCACACTA
GAGGCCTATTGCTACATCAAGGGGTTCCTCGAG

NOV55f, CG56258-03

| | | | |
|---|---|---|---|
| Protein Sequence | SEQ ID NO: 866 | 891 aa | MW at 98998.6kD |

EAGGSGDVPSTGQNNESCSGSSDCKEGVILPIWYPENPSLGDKIARVIVYFVALIYMFLGVSIIADRF
MASIEVITSQEREVTIKKPNGETSTTTIRVWNETVSNLTLMALGSSAPEILLSLIEVCGHGFIAGDLG
PSTIVGSAAFNMFIIIGICVYVIPDGETRKIKHLRVFFITAAWSIFAYIWLYMILAVFSPGVVQVWEG
LLTLFFFPVCVLLAWVADKRLLFYKYMHKKYRTDKHRGIIIETEGDHPKGIEMDGKMMNSHFLDGNLV
PLEGKEVDESRREMIRILKDLKQKHPEKDLDQLVEMANYYALSHQQKSRAFYRIQATRNMTGAGNILK
KHAAEQAKKASSMSEVHTDEPEDFISKVFFDPCSYQCLENCGAVLLTVVRKGGDMSKTMYVDYKTEDG
SANAGADYEFTEGTVVLKPGETQKEFSVGIIDDDIFEEDEHFFVRLSNVRIEEEQPEEGMPPAIFNSL
PLPRAVLASPCVATVTILDDDHAGIFTFECDTIHVSESIGVMEVKVLRTSGARGTVIVPFRTVEGTAK
GGGEDFEDTYGELEFKNDETVKTIRVKIVDEEEYERQENFFIALGEPKWMERGISDVTDRKLTMEEEE
AKRIAEMGKPVLGEHPKLEVIIEESYEFKTTVDKLIKKTNLALVVGTHSWRDQFMEAITVSAAGDEDE
DESGEERLPSCFDYVMHFLTVFWKVLFACVPPTEYCHGWACFAVSILlIGMLTAIIGDLASHFGCTIG
LKDSVTAVVFVAFGTSVPDTFASKAAALQDVYADASIGNVTGSNAVNVFLGIGLAWSVAAIYWALQGQ
EFHVSAGTLAFSVTLFTIFAFVCISVLLYRRRPHLGGELGGPRGCKLATTWLFVSLWLLYILFATLEA
YCYIKGF

| | | |
|---|---|---|
| NOV55g, CG56258-05 | SEQ ID NO: 867 | 2778 bp |

| | | |
|---|---|---|
| DNA Sequence | ORF Start: ATG at 10 | ORF Stop: at 2773 |

GGATCCACCATGGCGTGGTTAAGGTTGCAGCCTCTCACCTCTGCCTTCCTCCATTTTGGGCTGGTTAC
CTTTGTGCTCTTCCTGAATGGTCTTCGAGCAGAGGCTGGTGGCTCAGGGGACGTGCCAAGCACAGGGC
AGAACAATGAGTCCTGTTCAGGGTCATCGGACTGCAAGGAGGGTGTCATCCTGCCAATCTGGTACCCG
GAGAACCCTTCCCTTGGGGACAAGATTGCCAGGGTCATTGTCTATTTTGTGGCCCTGATATACATGTT
CCTTGGGGTGTCCATCATTGCTGACCGCTTCATGGCATCTATTGAAGTCATCACCTCTCAAGAGAGGG
AGGTGACAATTAAGAAACCCAATGGAGAAACCAGCACAACCACTATTCGGGTCTGGAATGAAACTGTC
TCCAACCTGACCCTTATGGCCCTGGGTTCCTCTGCTCCTGAGATACTCCTCTCTTTAATTGAGGTGTG
TGGTCATGGGTTCATTGCTGGTGATCTGGGACCTTCTACCATTGTAGGGAGTGCAGCCTTCAACATGT
TCATCATCATTGGCATCTGTGTCTACGTGATCCCAGACGGAGAGACTCGCAAGATCAAGCATCTACGA
GTCTTCTTCATCACCGCTGCTTGGAGTATCTTTGCCTACATCTGGCTCTATATGATTCTGGCAGTCTT
CTCCCCTGGTGTGGTCCAGGTTTGGGAAGGCCTCCTCACTCTCTTCTTCTTTCCAGTGTGTGTCCTTC
TGGCCTGGGTGGCAGATAAACGACTGCTCTTCTACAAATACATGCACAAAAAGTACCGCACAGACAAA
CACCGAGGAATTATCATAGAGACAGAGGGTGACCACCCTAAGGGCATTGAGATGGATGGGAAAATGAT
GAATTCCCATTTTCTAGATGGGAACCTGGTGCCCCTGGAAGGGAAGGAAGTGGATGAGTCCCGCAGAG
AGATGATCCGGATTCTCAAGGATCTGAAGCAAAAACACCCAGAGAAGGACTTAGATCAGCTGGTGGAG
ATGGCCAATTACTATGCTCTTTCCCACCAACAGAAGAGCCGCGCCTTCTACCGTATCCAAGCCACTCG
TATGATGACTGGTGCAGGCAATATCCTGAAGAAACATGCAGCAGAACAAGCCAAGAAGGCCTCCAGCA
TGAGCGAGGTGCACACCGATGAGCCTGAGGACTTTATTTCCAAGGTCTTCTTTGACCCATGTTCTTAC
CAGTGCCTGGAGAACTGTGGGGCTGTACTCCTGACAGTGGTGAGGAAAGGGGGAGACATGTCAAAGAC
CATGTATGTGGACTACAAAACAGAGGATGGTTCTGCCAATGCAGGGGCTGACTATGAGTTCACAGAGG
GCACGGTGGTTCTGAAGCCAGGAGAGACCCAGAAGGAGTTCTCCGTGGGCATAATTGATGACGACATT
TTTGAGGAGGATGAACACTTCTTTGTAAGGTTGAGCAATGTCCGCATAGAGGAGGAGCAGCCAGAGGA
GGGGATGCCTCCAGCAATATTCAACAGTCTTCCCTTGCCTCGGGCTGTCCTAGCCTCCCCTTGTGTGG
CCACAGTTACCATCTTGGATGATGACCATGCAGGCATCTTCACTTTTGAATGTGATACTATTCATGTC
AGTGAGAGTATTGGTGTTATGGAGGTCAAGGTTCTGCGGACATCAGGTGCCCGGGGTACAGTCATCGT
CCCCTTTAGGACAGTAGAAGGGACAGCCAAGGGTGGCGGTGAGGACTTTGAAGACACATATGGGGAGT
TGGAATTCAAGAATGATGAAACTGTGAAAACCATAAGGGTTAAAATAGTAGATGAGGAGGAATACGAA
AGGCAAGAGAATTTCTTCATTGCCCTTGGTGAACCGAAATGGATGGAACGTGGAATATCAGATGTGAC
AGACAGGAAGCTGACTATGGAAGAAGAGGAGGCCAAGAGGATAGCAGAGATGGGAAAGCCAGTATTGG
GTGAACACCCCAAACTAGAAGTCATCATTGAAGAGTCCTATGAGTTCAAGACTACGGTGGACAAACTG
ATCAAGAAGACAAACCTGGCCTTGGTTGTGGGGACCCATTCCTGGAGGGACCAGTTCATGGAGGCCAT
CACCGTCAGTGCAGCAGGGGATGAGGATGAGGATGAATCCGGGGAGGAGAGGCTGCCCTCCTGCTTTG
ACTACGTCATGCACTTCCTGACTGTCTTCTGGAAGGTGCTGTTTGCCTGTGTGCCCCCCACAGAGTAC
TGCCACGGCTGGGCCTGCTTCGCCGTCTCCATCCTCATCATTGGCATGCTCACCGCCATCATTGGGGA
CCTGGCCTCGCACTTCGGCTGCACCATTGGTCTCAAAGATTCAGTCACAGCTGTTGTTTTCGTGGCAT
TTGGCACCTCTGTCCCAGATACGTTTGCCAGCAAAGCTGCTGCCCTCCAGGATGTATATGCAGACGCC
TCCATTGGCAACGTGACGGGCAGCAACGCCGTCAATGTCTTCCTGGGCATCGGCCTGGCCTGGTCCGT
GGCCGCCATCTACTGGGCTCTGCAGGGACAGGAGTTCCACGTGTCGGCCGGCACACTGGCCTTCTCCG
TCACCCTCTTCACCATCTTTGCATTTGTCTGCATCAGCGTGCTCTTGTACCGAAGGCGGCCGCACCTG
GGAGGGGAGCTTGGTGGCCCCCGTGGCTGCAAGCTCGCCACAACATGGCTCTTTGTGAGCCTGTGGCT
CCTCTACATACTCTTTGCCACACTAGAGGCCTATTGCTACATCAAGGGGTTCCTCGAG

NOV55g, CG56258-05

| | | | |
|---|---|---|---|
| Protein Sequence | SEQ ID NO: 868 | 921 aa | MW at 102413.7kD |

MAWLRLQPLTSAFLHFGLVTFVLFLNGLRAEAGGSGDVPSTGQNNESCSGSSDCKEGVILPIWYPENP
SLGDKIARVIVYFVALIYMFLGVSIIADRFMASIEVITSOEREVTIKKPNGETSTTTIRVWNETVSNL
TLMALGSSAPEILLSLIEVCGHGFIAGDLGPSTIVGSAAFNMFIIIGICVYVIPDGETRKIKHLRVFF
ITAAWSIFAYIWLYMILAVFSPGVVQVWEGLLTLFFFPVCVLIAWVADKRLLFYKYMHKKYRTDKHRG
IIIETEGDHPKGIEMDGKMMNSHFLDGNLVPLEGKEVDESRREMIRILKDLKQKHPEKDLDQLVEMAN
YYALSHQQKSRAFYRIQATRMMTGAGNILKKHAAEQAKKASSMSEVHTDEPEDFISKVFFDPCSYQCL
ENCGAVLLTVVRKGGDMSKTMYVDYKTEDGSANAGADYEFTEGTVVLKPGETQKEFSVGIIDDDIFEE
DEHFFVRLSNVRIEEEQPEEGMPPAIFNSLPLPRAVLASPCVATVTILDDDHAGIFTFECDTIHVSES
IGVMEVKVLRTSGARGTVIVPFRTVEGTAKGGGEDFEDTYGELEFKNDETVKTIRVKIVDEEEYERQE
NFFIALGEPKWMERGISDVTDRKLTMEEEEAKRIAEMGKPVLGEHPKLEVIIEESYEFKTTVDKLIKK
TNLALVVGTHSWRDQFMEAITVSAAGDEDEDESGEERLPSCFDYVMHFLTVFWKVLFACVPPTEYCHG
WACFAVSILIIGMLTAIIGDLASHFGCTIGLKDSVTAVVFVAFGTSVPDTFASKAAALQDVYADASIG
NVTGSNAVNVFLGIGLAWSVAAIYWALQGQEFHVSAGTLAFSVTLFTIFAFVCISVLLYRRRPHLGGE
LGGPRGCKLATTWLFVSLWLLYILFATLEAYCYIKGF

| | | |
|---|---|---|
| NOV55h, CG56258-06 | SEQ ID NO: 869 | 2813 bp |

| | | |
|---|---|---|
| DNA Sequence | ORF Start: ATG at 9 | ORF Stop: TAG at 2793 |

TCTCGTGTATGGCGTGGTTAAGGTTGCAGCCTCTCACCTCTGCCTTCCTCCATTTTGGGCTGGTTACC
TTTGTGCTCTTCCTGAATGGTCTTCGAGCAGAGGCTGGTGGCTCAGGGGACGTGCCAAGCACAGGGCA
GAACAATGAGTCCTGTTCAGGGTCATCGGACTGCAAGGAGGGTGTCATCCTGCCAATCTGGTACCCGG
AGAACCCTTCCCTTGGGGACAAGATTGCCAGGGTCATTGTCTATTTTGTGGCCCTGATATACATGTTC
CTTGGGGTGTCCATCATTGCTGACCGCTTCATGGCATCTATTGAAGTCATCACCTCTCAAGAGAGGGA
GGTGACAATTAAGAAACCCAATGGAGAAACCAGCACAACCACTATTCGGGTCTGGAATGAAACTGTCT
CCAACCTGACCCTTATGGCCCTGGGTTCCTCTGCTCCTGAGATACTCCTCTCTTTAATTGAGGTGTGT
GGTCATGGGTTCATTGCTGGTGATCTGGGACCTTCTACCATTGTAGGGAGTGCAGCCTTCAACATGTT
CATCATCATTGGCATCTGTGTCTACGTGATCCCAGACGGAGAGACTCGCAAGATCAAGCATCTACGAG
TCTTCTTCATCACCGCTGCTTGGAGTATCTTTGCCTACATCTGGCTCTATATGATTCTGGCAGTCTTC
TCCCCTGGTGTGGTCCAGGTTTGGGAAGGCCTCCTCACTCTCTTCTTCTTTCCAGTGTGTGTCCTTCT
GGCCTGGGTGGCAGATAAACGACTGCTCTTCTACAAATACATGCACAAAAAGTACCGCACAGACAAAC
ACCGAGGAATTATCATAGAGACAGAGGGTGACCACCCTAAGGGCATTGAGATGGATGGGAAAATGATG
AATTCCCATTTTCTAGATGGGAACCTGGTGCCCCTGGAAGGGAAGGAAGTGGATGAGTCCCGCAGAGA
GATGATCCGGATTCTCAAGGATCTGAAGCAAAAACACCCAGAGAAGGACTTAGATCAGCTGGTGGAGA
TGGCCAATTACTATGCTCTTTCCCACCAACAGAAGAGCCGTGCCTTCTACCGTATCCAAGCCACTCGT
ATGATGACTGGTGCAGGCAATATCCTGAAGAAACATGCAGCAGAACAAGCCAAGAAGGCCTCCAGCAT
GAGCGAGGTGCACACCGATGAGCCTGAGGACTTTATTTCCAAGGTCTTCTTTGACCCATGTTCTTACC
AGTGCCTGGAGAACTGTGGGGCTGTACTCCTGACAGTGGTGAGGAAAGGGGGAGACATGTCAAAGACC
ATGTATGTGGACTACAAAACAGAGGATGGTTCTGCCAATGCAGGGGCTGACTATGAGTTCACAGAGGG
CACGGTGGTTCTGAAGCCAGGAGAGACCCAGAAGGAGTTCTCCGTGGGCATAATTGATGACGACATTT
TTGAGGAGGATGAACACTTCTTTGTAAGGTTGAGCAATGTCCGCATAGAGGAGGAGCAGCCAGAGGAG
GGGATGCCTCCAGCAATATTCAACAGTCTTCCCTTGCCTCGGGCTGTCCTAGCCTCCCCTTGTGTGGC
CACAGTTACCATCTTGGATGATGACCATGCAGGCATCTTCACTTTTGAATGTGATACTATTCATGTCA
GTGAGAGTATTGGTGTTATGGAGGTCAAGGTTCTGCGGACATCAGGTGCCCGGGGTACAGTCATCGTC
CCCTTTAGGACAGTAGAAGGGACAGCCAAGGGTGGCGGTGAGGACTTTGAAGACACATATGGGGAGTT
GGAATTCAAGAATGATGAAACTGTGAAAACTCTTCAGGTGAAGATAGTTGATGACGAGGAATATGAGA
AAAAGGATAATTTCTTCATTGAGCTGGGCCAGCCCCAGTGGCTTAAGCGAGGGATTTCAGCTCTGCTA
CTCAATCAAGGGGATGGGGACAGGAAGCTAACAGCCGAGGAGGAGGAGGCTCGGAGGATAGCAGAGAT
GGGCAAGCCAGTTCTTGGGGAGAACTGCCGGCTGGAGGTCATCATCGAGGAGTCATATGATTTTAAGA
ACACGGTGGATAAACTCATCAAGAAAACGAACTTGGCCTTGGTAATTGGGACCCATTCATGGAGGGAG
CAGTTTTTAGAGGCAATTACGGTGAGCGCAGGGGACGAGGAGGAGGAGGAGGACGGGTCCCGGGAGGA
GCGGCTGCCGTCGTGCTTTGACTACGTGATGCACTTCCTGACGGTGTTCTGGAAGGTGCTCTTCGCCT
GTGTGCCCCCCACCGAGTACTGCCACGGCTGGGCCTGCTTTGGTGTCTCCATCCTGGTCATCGGCCTG
CTCACCGCCCTCATTGGGGACCTCGCCTCCCACTTCGGCTGCACCGTTGGCCTCAAGGACTCTGTCAA
TGCTGTTGTCTTCGTTGCCCTGGGCACCTCCATCCCTGACACGTTCGCCAGCAAGGTGGCGGCGCTGC
AGGACCAGTGCGCCGACGCGTCCATCGGCAACGTGACCGGCTCCAACGCGGTGAACGTGTTCCTTGGC
CTGGGCGTCGCCTGGTCTGTGGCCGCCGTGTACTGGGCGGTGCAGGGCCGCCCCTTCGAGGTGCGCAC
TGGCACGCTGGCCTTCTCCGTCACGCTCTTCACCGTCTTCGCCTTCGTGGGCATTGCCGTGCTGCTGT
ACCGGCGCCGGCCGCACATCGGCGGCGAGCTGGGCGGCCCGCGCGGACCCAAGCTCGCCACCACCGCG
CTCTTCCTGGGCCTCTGGCTCCTGTACATCCTCTTCGCCAGCCTGGAGGCGTACTGCCACATCCGGGG
CTTCTAGGGCCTCGCGCAGAGACTC

NOV55h, CG56258-06

| | | | |
|---|---|---|---|
| Protein Sequence | SEQ ID NO: 870 | 928 aa | MW at 102900.1kD |

MAWLRLQPLTSAFLHFGLVTFVLFLNGLRAEAGGSGDVPSTGQNNESCSGSSDCKEGVILPIWYPENP
SLGKDIARVIVYFVALIYMFLGVSIIADRFMASIEVITSQEREVTIKKPNGETSTTTIRVWNETVSNL
TLMALGSSAPEILLSLIEVCGHGFIAGDLGPSTIVGSAAFNMFIIIGICVYVIPDGETRKIKHLRVFF
ITAAWSIFAYIWLYMILAVFSPGVVQVWEGLLTLFFFPVCVLLAWVADKRLLFYKYMHKKYRTDKHRG
IIIETEGDHPKGIEMDGKMMNSHFLDGNLVPLEGKEVDESRREMIRILKDLKQKHPEKDLDQLVEMAN
YYALSHQQKSRAFYRIQATRMMTGAGNILKKHAAEQAKKASSMSEVHTDEPEDFISKVFFDPCSYQCL
ENCGAVLLTVVRKGGDMSKTMYVDYKTEDGSANAGADYEFTEGYVVLKPGETQKEFSVGIIDDDIFEE
DEHFFVRLSNVRIEEEQPEEGMPPAIFNSLPLPRAVLASPCVATVTILDDDHAGIFTFECDTIHVSES
IGVMEVKVLRTSGARGTVIVPFRTVEGTAKGGGEDFEDTYGELEFKNDETVKTLQVKIVDDEEYEKKD
NFFIELGQPQWLKRGISALLLNQGDGDRKLTAEEEEARRIAEMGKPVLGENCRLEVIIEESYDFKNTV
DKLIKKTNLALVIGTHSWREQFLEAITVSAGDEEEEEDGSREERLPSCFDYVMHFLTVFWKVLFACVP
PTEYCHGWACFGVSILVIGLLTALIGDLASHFGCTVGLKDSVNAVVFVALGTSIPDTFASKVAALQDQ
CADASIGNVTGSNAVNVFLGLGVAWSVAAVYWAVQGRPFEVRTGTLAFSVTLFTVFAFVGIAVLLYRR
RPHIGGELGGPRGPKLATTALFLGLWLLYILFASLEAYCHIRGF

A ClustalW comparison of the above protein sequences yields the following sequence alignment shown in Table 55B.

TABLE 55B
Comparison of the NOV55 protein sequences.

| | |
|---|---|
| NOV55a | ---MAWLRLQPLTSAFLHFGLVTFVLFNGLRAEAGGSGDVPSTGQNNESCSGSSDCKEG |
| NOV55b | ---MAWLRLQPLTSAFLHFGLVTFVLFNGLRAEAGGSGDVPSTGQNNESCSGSSDCKEG |
| NOV55c | GSTMAWLRLQPLTSAFLHFGLVTFVLFNGLRAEAGGSGDVPSTGQNNESCSGSSDCKEG |
| NOV55d | -------------------------------GSEAGGSGDVPSTGQNNESCSGSSDCKEG |
| NOV55e | ---MAWLRLQPLTSAFLHFGLVTFVLFNGLRAEAGGSGDVPSTGQNNESCSGSSDCKEG |
| NOV55f | ---------------------------------EAGGSGDVPSTGQNNESCSGSSDCKEG |
| NOV55g | ---MAWLRLQPLTSAFLHFGLVTFVLFNGLRAEAGGSGDVPSTGQNNESCSGSSDCKEG |
| NOV55h | ---MAWLRLQPLTSAFLHFGLVTFVLFNGLRAEAGGSGDVPSTGQNNESCSGSSDCKEG |
| NOV55a | VILPIWYPENPSLGDKIARVIVYFVALIYMFLGVSIIADRFMASIEVITSQEREVTIKKP |
| NOV55b | VILPIWYPENPSLGDKIARVIVYFVALIYMFLGVSIIADRFMASIEVITSQEREVTIKKP |
| NOV55c | VILPIWYPENPSLGDKIARVIVYFVALIYMFLGVSIIADRFMASIEVITSQEREVTIKKP |
| NOV55d | VILPIWYPENPSLGDKIARVIVYFVALIYMFLGVSIIADRFMASIEVITSQEREVTIKKP |
| NOV55e | VILPIWYPENPSLGDKIARVIVYFVALIYMFLGVSIIADRFMASIEVITSQEREVTIKKP |
| NOV55f | VILPIWYPENPSLGDKIARVIVYFVALIYMFLGVSIIADRFMASIEVITSQEREVTIKKP |
| NOV55g | VILPIWYPENPSLGDKIARVIVYFVALIYMFLGVSIIADRFMASIEVITSQEREVTIKKP |
| NOV55h | VILPIWYPENPSLGDKIARVIVYFVALIYMFLGVSIIADRFMASIEVITSQEREVTIKKP |
| NOV55a | NGETSTTTIRVWNETVSNLTLMALGSSAPEILLSLIEVCGHGFIAGDLGPSTIVGSAAFN |
| NOV55b | NGETSTTTIRVWNETVSNLTLMALGSSAPEILLSLIEVCGHGFIAGDLGPSTIVGSAAFN |
| NOV55c | NGETSTTTIRVWNETVSNLTLMALGSSAPEILLSLIEVCGHGFIAGDLGPSTIVGSAAFN |
| NOV55d | NGETSTTTIRVWNETVSNLTLMALGSSAPEILLSLIEVCGHGFIAGDLGPSTIVGSAAFN |
| NOV55e | NGETSTTTIRVWNETVSNLTLMALGSSAPEILLSLIEVCGHGFIAGDLGPSTIVGSAAFN |
| NOV55f | NGETSTTTIRVWNETVSNLTLMALGSSAPEILLSLIEVCGHGFIAGDLGPSTIVGSAAFN |
| NOV55g | NGETSTTTIRVWNETVSNLTLMALGSSAPEILLSLIEVCGNGFIAGDLGPSTIVGSAAFN |
| NOV55h | NGETSTTTIRVWNETVSNLTLMALGSSAPEILLSLIEVCGHGFIAGDLGPSTIVGSAAFN |
| NOV55a | MFIIIGICVYVIPDGETRKIKHLRVFFITAAWSIFAYIWLYMILAVFSPGVVQVWEGLLT |
| NOV55b | MFIIIGICVYVIPDGETRKIKHLRVFFITAAWSIFAYIWLYMILAVFSPGVVQVWEGLLT |
| NOV55c | MFIIIGICVYVIPDGETRKIKHLRVFFITAAWSIFAYIWLYMILAVFSPGVVQVWEGLLT |
| NOV55d | MFIIIGICVYVIPDGETRKIKHLRVFFITAAWSIFAYIWLYMILAVFSPGVVQVWEGLLT |
| NOV55e | MFIIIGICVYVIPDGETRKIKHLRVFFITAAWSIFAYIWLYMILAVFSPGVVQVWEGLLT |
| NOV55f | MFIIIGICVYVIPDGETRKIKHLRVFFITAAWSIFAYIWLYMILAVFSPGVVQVWEGLLT |
| NOV55g | MFIIIGICVYVIPDGETRKIKHLRVFFITAAWSIFAYIWLYMILAVFSPGVVQVWEGLLT |
| NOV55h | MFIIIGICVYVIPDGETRKIKHLRVFFITAAWSIFAYIWLYMILAVFSPGVVQVWEGLLT |
| NOV55a | LFFFPVCVLLAWVADKRLLFYKYMHKKYRTDKHRGIIIETEGDHPKGIEMDGKMMNSHFL |
| NOV55b | LFFFPVCVLLAWVADKRLLFYKYMHKKYRTDKHRGIIIETEGDHPKGIEMDGKMMNSHFL |
| NOV55c | LFFFPVCVLLAWVADKRLLFYKYMHKKYRTDKHRGIIIETEGDHPKGIEMDGKMMNSHFL |
| NOV55d | LFFFPVCVLLAWVADKRLLFYKYMHKKYRTDKHRGIIIETEGDHPKGIEMDGKMMNSHFL |
| NOV55e | LFFFPVCVLLAWVADKRLLFYKYMHKKYRTDKHRGIIIETEGDHPKGIEMDGKMMNSHFL |
| NOV55f | LFFFPVCVLLAWVADKRLLFYKYMHKKYRTDKHRGIIIETEGDHPKGIEMDGKMMNSHFL |
| NOV55g | LFFFPVCVLLAWVADKRLLFYKYMHKKYRTDKHRGIIIETEGDHPKGIEMDGKMMNSHFL |
| NOV55h | LFFFPVCVLLAWVADKRLLFYKYMHKKYRTDKHRGIIIETEGDHPKGIEMDGKMMNSHFL |
| NOV55a | DGNLVPLEGKEVDESRREMIRILKDLKQKHPEKDLDQLVEMANYYALSHQQKSRAFYRIQ |
| NOV55b | DGNLVPLEGKEVDESRREMIRILKDLKQKHPEKDLDQLVEMANYYALSHQQKSRAFYRIQ |
| NOV55c | DGNLVPLEGKEVDESRREMIRILKDLKQKHPEKDLDQLVEMANYYALSHQQKSRAFYRIQ |
| NOV55d | DGNLVPLEGKEVDESRREMIRILKDLKQKHPEKDLDQLVEMANYYALSHQQKSRAFYRIQ |
| NOV55e | DGNLVPLEGKEVDESRREMIRILKDLKQKHPEKDLDQLVEMANYYALSHQQKSRAFYRIQ |
| NOV55f | DGNLVPLEGKEVDESRREMIRILKDLKQKHPEKDLDQLVEMANYYALSHQQKSRAFYRIQ |
| NOV55g | DGNLVPLEGKEVDESRREMIRILKDLKQKHPEKDLDQLVEMANYYALSHQQKSRAFYRIQ |
| NOV55h | DGNLVPLEGKEVDESRREMIRILKDLKQKHPEKDLDQLVEMANYYALSHQQKSRAFYRIQ |
| NOV55a | ATRMMTGAGNILKKHAAEQAKKASSMSEVHTDEPEDFISKVFFDPCSYQCLENCGAVLLT |
| NOV55b | ATRMMTGAGNILKKHAAEQAKKASSMSEVHTDEPEDFISKVFFDPCSYQCLENCGAVLLT |
| NOV55c | ATRMMTGAGNILKKHAAEQAKKASSMSEVHTDEPEDFISKVFFDPCSYQCLENCGAVLLT |
| NOV55d | ATRMMTGAGNILKKHAAEQAKKASSMSEVHTDEPEDFISKVFFDPCSYQCLENCGAVLLT |
| NOV55e | ATRMMTGAGNILKKHAAEQAKKASSMSEVHTDEPEDFISKVFFDPCSYQCLENCGAVLLT |
| NOV55f | ATRMMTGAGNILKKHAAEQAKKASSMSEVHTDEPEDFISKVFFDPCSYQCLENCGAVLLT |
| NOV55g | ATRMMTGAGNILKKHAAEQAKKASSMSEVHTDEPEDFISKVFFDPCSYQCLENCGAVLLT |
| NOV55h | ATRMMTGAGNILKKHAAEQAKKASSMSEVHTDEPEDFISKVFFDPCSYQCLENCGAVLLT |
| NOV55a | VVRKGGDMSKTMYVDYKTEDGSANAGADYEFTEGTVVLKPGETQKEFSVGIIDDDIFEED |
| NOV55b | VVRKGGDMSKTMYVDYKTEDGSANAGADYEFTEGTVVLKPGETQKEFSVGIIDDDIFEED |
| NOV55c | VVRKGGDMSKTMYVDYKTEDGSANAGADYEFTEGTVVLKPGETQKEFSVGIIDDDIFEED |
| NOV55d | VVRKGGDMSKTMYVDYKTEDGSANAGADYEFTEGTVVLKPGETQKEFSVGIIDDDIFEED |
| NOV55e | VVRKGGDMSKTMYVDYKTEDGSANAGADYEFTEGTVVLKPGETQKEFSVGIIDDDIFEED |
| NOV55f | VVRKGGDMSKTMYVDYKTEDGSANAGADYEFTEGTVVLKPGETQKEFSVGIIDDDIFEED |
| NOV55g | VVRKGGDMSKTMYVDYKTEDGSANAGADYEFTEGTVVLKPGETQKEFSVGIIDDDIFEED |
| NOV55h | VVRKGGDMSKTMYVDYKTEDGSANAGADYEFTEGTVVLKPGETQKEFSVGIIDDDIFEED |
| NOV55a | EHFFVRLSNVRIEEEQPEEGMPPAIFNSLPLPRAVLASPCVATVTILDDDHAGIFTFECD |
| NOV55b | EHFFVRLSNVRIEEEQPEEGMPPAIFNSLPLPRAVLASPCVATVTILDDDHAGIFTFECD |
| NOV55c | EHFFVRLSNVRIEEEQPEEGMPPAIFNSLPLPRAVLASPCVATVTILDDDHAGIFTFECD |
| NOV55d | EHFFVRLSNVRIEEEQPEEGMPPAIFNSLPLPRAVLASPCVATVTILDDDHAGIFTFECD |
| NOV55e | EHFFVRLSNVRIEEEQPEEGMPPAIFNSLPLPRAVLASPCVATVTILDDDHAGIFTFECD |
| NOV55f | EHFFVRLSNVRIEEEQPEEGMPPAIFNSLPLPRAVLASPCVATVTILDDDHAGIFTFECD |
| NOV55g | EHFFVRLSNVRIEEEQPEEGMPPAIFNSLPLPRAVLASPCVATVTILDDDHAGIFTFECD |
| NOV55h | EHFFVRLSNVRIEEEQPEEGMPPAIFNSLPLPRAVLASPCVATVTILDDDHAGIFTFECD |
| NOV55a | TIHVSESIGVMEVKVLRTSGARGTVIVPFRTVEGTAKGGGEDFEDTYGELEFKNDETVKT |
| NOV55b | TIHVSESIGVMEVKVLRTSGARGTVIVPFRTVEGTAKGGGEDFEDTYGELEFKNDETVKT |
| NOV55c | TIHVSESIGVMEVKVLRTSGARGTVIVPFRTVEGTAKGGGEDFEDTYGELEFKNDETVKT |
| NOV55d | TIHVSESIGVMEVKVLRTSGARGTVIVPFRTVEGTAKGGGEDFEDTYGELEFKNDETVKT |
| NOV55e | TIHVSESIGVMEVKVLRTSGARGTVIVPFRTVEGTAKGGGEDFEDTYGELEFKNDETVKT |
| NOV55f | TIHVSESIGVMEVKVLRTSGARGTVIVPFRTVEGTAKGGGEDFEDTYGELEFKNDETVKT |
| NOV55g | TIHVSESIGVMEVKVLRTSGARGTVIVPFRTVEGTAKGGGEDFEDTYGELEFKNDETVKT |
| NOV55h | TIHVSESIGVMEVKVLRTSGARGTVIVPFRTVEGTAKGGGEDFEDTYGELEFKNDETVKT |
| NOV55a | IRVKIVDEEEYERQENFFIALGEP----KWMERGISDVT---DRKLTMEEEEAKRIAEMG |
| NOV55b | IHIKVIDDEAYEKNKNYFIEMMGPRMVDMSFQKALLLSP---DRKLTMEEEEAKRIAEMG |
| NOV55c | IRVKIVDEEEYERQENFFIALGEP----KWMERGISDVT---DRKLTMEEEEAKRIAEMG |
| NOV55d | IRVKIVDEEEYERQENFFIALGEP----KWMERGISDVT---DRKLTMEEEEAKRIAEMG |
| NOV55e | LQVKIVDDEEYEKKDNFFIELGQPQ-WLKRGISALLLNQGDGDRKLTAEEEEARRIAEMG |
| NOV55f | IRVKIVDEEEYERQENFFIALGEP----KWMERGISDVT---DRKLTMEEEEAKRIAEMG |
| NOV55g | IRVKIVDEEEYERQENFFIALGEP----KWMERGISDVT---DRKLTMEEEEAKRIAEMG |
| NOV55h | LQVKIVDDEEYEKKDNFFIELGQPQ-WLKRGISALLLNQGDGDRKLTAEEEEARRIAEMG |
| NOV55a | KPVLGEHPKLEVIIEESYEFKTTVDKLIKKTNLALVVGTHSWRDQFMEAITVSAAGDEDE |
| NOV55b | KPVLGEHPKLEVIIEESYEFKTTVDKLIKKTNLALVVGTHSWRDQFMEAITVSAAGDEDE |
| NOV55c | KPVLGEHPKLEVIIEESYEFKTTVDKLIKKTNLALVVGTHSWRDQFMEAITVSAAGDEDE |
| NOV55d | KPVLGEHPKLEVIIEESYEFKTTVDKLIKKTNLALVVGTHSWRDQFMEAITVSAAGDEDE |
| NOV55e | KPVLGENCKLEVIIEESYDFKNTVDKLIKKTNLALVIGTHSWREQFLEAITVSAG-DEEE |
| NOV55f | KPVLGEHPKLEVIIEESYEFKTTVDKLIKKTNLALVVGTHSWRDQFMEAITVSAAGDEDE |
| NOV55g | KPVLGEHPKLEVIIEESYEFKTTVDKLIKKTNLALVVGTHSWRDQFMEAITVSAAGDEDE |
| NOV55h | KPVLGENCKLEVIIEESYDFKNTVDKLIKKTNLALVIGTHSWREQFLEAITVSAG-DEEE |
| NOV55a | DESG--EERLPSCFDYVMHFLTVFWKVLFACVPPTEYCHGWACFAVSILIIGMLTAIIGD |
| NOV55b | DESG--EERLPSCFDYVMHFLTVFWKVLFACVPPTEYCHGWACFAVSILIIGMLTAIIGD |
| NOV55c | DESG--EERLPSCFDYVMHFLTVFWKVLFACVPPTEYCHGWACFAVSILIIGMLTAIIGD |
| NOV55d | DESG--EERLPSCFDYVMHFLTVFWKVLFACVPPTEYCHGWACFAVSILIIGMLTAIIGD |
| NOV55e | EEDGSREERLPSCFDYVMHFLTVFWKVLFACVPPTEYCHGWACFAVSILIIGMLTAIIGD |
| NOV55f | DESG--EERLPSCFDYVMHFLTVFWKVLFACVPPTEYCHGWACFAVSILIIGMLTAIIGD |
| NOV55g | DESG--EERLPSCFDYVMHFLTVFWKVLFACVPPTEYCHGWACFAVSILIIGMLTAIIGD |
| NOV55h | EEDGSREERLPSCFDYVMHFLTVFWKVLFACVPPTEYCHGWACFAVSILIIGMLTAIIGD |
| NOV55a | LASHFGCTIGLKDSVTAVVFVAFGTSVPDTFASKAAALQDVYADASIGNVTGSNAVNVFL |
| NOV55b | LASHFGCTIGLKDSVTAVVFVAFGTSVPDTFASKAAALQDVYADASIGNVTGSNAVNVFL |
| NOV55c | LASHFGCTIGLKDSVTAVVFVAFGTSVPDTFASKAAALQDVYADASIGNVTGSNAVNVFL |
| NOV55d | LASHFGCTIGLKDSVTAVVFVAFGTSVPDTFASKAAALQDVYADASIGNVTGSNAVNVFL |
| NOV55e | LASHFGCTVGLKDSVNAVVFVALGTSIPDTFASKVAALQDQCADASIGNVTGSNAVNVFL |
| NOV55f | LASHFGCTIGLKDSVTAVVFVAFGTSVPDTFASKAAALQDVYADASIGNVTGSNAVNVFL |
| NOV55g | LASHFGCTIGLKDSVTAVVFVAFGTSVPDTFASKAAALQDVYADASIGNVTGSNAVNVFL |
| NOV55h | LASHFGCTVGLKDSVNAVVFVALGTSIPDTFASKVAALQDQCADASIGNVTGSNAVNVFL |
| NOV55a | GIGLAWSVAAIYWALQGQEFHVSAGTLAFSVTLFTIFAFVCICVLLYRRRPHLGGELGGP |
| NOV55b | GIGLAWSVAAIYWALQGQEFHVSAGTLAFSVTLFTIFAFVCICVLLYRRRPHLGGELGGP |
| NOV55c | GIGLAWSVAAIYWALQGQEFHVSAGTLAFSVTLFTIFAFVCICVLLYRRRPHLGGELGGP |
| NOV55d | GIGLAWSVAAIYWALQGQEFEVSAGTLAFSVTLFTIFAFVCICVLLYRRRPHLGGELGGP |
| NOV55e | GLGVAWSVAAVYWALQGRPFHVSARTTAFSVTLFTVFAFVGIAVLLYRRRPHIGGELGGP |
| NOV55f | GIGLAWSVAAIYWALQGQEFHVSAGTLAFSVTLFTIFAFVCICVLLYRRRPHLGGELGGP |
| NOV55g | GIGLAWSVAAIYWALQGQEFHVSAGTLAFSVTLFTIFAFVCICVLLYRRRPHLGGELGGP |
| NOV55h | GLGVAWSVAAVYWALQGRPFEVSARTTAFSVTLFTVFAFVGIAVLLYRRRPHIGGELGGP |
| NOV55a | RGCKLATTWLFVSLWLLYILFATLEAYCYIKGF-- |
| NOV55b | RGCKLATTWLFVSLWLLYILFATLEAYCYIKGF-- |
| NOV55c | RGCKLATTWLFVSLWLLYILFATLEAYCYIKGFLE |
| NOV55d | RGCKLATTWLFVSLWLLYILFATLEAYCYIKGFLE |
| NOV55e | RGPKLATTALFLGLWLLYILFASLEAYCHIRGF-- |
| NOV55f | RGCKLATTWLFVSLWLLYILFATLEAYCYIKGF-- |
| NOV55g | RGCKLATTWLFVSLWLLYILFATLEAYCYIKGF-- |
| NOV55h | RGPKLATTALFLGLWLLYILFASLEAYCHIRGF-- |
| NOV55a | (SEQ ID NO: 856) |
| NOV55b | (SEQ ID NO: 858) |
| NOV55C | (SEQ ID NO: 860) |
| NOV55d | (SEQ ID NO: 862) |
| NOV55e | (SEQ ID NO: 864) |
| NOV55f | (SEQ ID NO: 866) |
| NOV55g | (SEQ ID NO: 868) |
| NOV55h | (SEQ ID NO: 870) |

Further analysis of the NOV55a protein yielded the following properties shown in Table 55C.

TABLE 55C
Protein Sequence Properties NOV55a

| | |
|---|---|
| SignalP analysis: | Cleavage site between residues 31 and 32 |
| PSORT II analysis: | |

| | |
|---|---|
| PSG: | a new signal peptide prediction method |
| | N-region: length 5; pos.chg 1; neg.chg 0 |
| | H-region: length 23; peak value 11.61 |
| | PSG score: 7.21 |
| GvH: | von Heijne's method for signal seq. recognition |
| | GvH score (threshold: −2.1): 0.67 |
| | possible cleavage site: between 30 and 31 |

>>> Seems to have a cleavable signal peptide (1 to 30)
ALOM: Klein et al's method for TM region allocation

| Init position for calculation: 31 | | |
|---|---|---|
| Tentative number of TMS(s) for the threshold 0.5: 11 | | |
| INTEGRAL | Likelihood = −8.92 | Transmembrane 78-94 |
| INTEGRAL | Likelihood = −6.64 | Transmembrane 170-186 |
| INTEGRAL | Likelihood = −3.24 | Transmembrane 208-224 |
| INTEGRAL | Likelihood = −8.49 | Transmembrane 235-251 |
| INTEGRAL | Likelihood = 0.21 | Transmembrane 508-524 |
| INTEGRAL | Likelihood = −1.12 | Transmembrane 724-740 |
| INTEGRAL | Likelihood = −9.61 | Transmembrane 750-766 |
| INTEGRAL | Likelihood = 0.05 | Transmembrane 775-791 |
| INTEGRAL | Likelihood = −3.35 | Transmembrane 823-839 |
| INTEGRAL | Likelihood = −8.39 | Transmembrane 858-874 |
| INTEGRAL | Likelihood = −3.56 | Transmembrane 893-909 |

| | |
|---|---|
| PERIPHERAL | Likelihood = 1.06 (at 137) |

| | ALOM score: −9.61 (number of TMSs: 11) |
|---|---|
| MTOP: | Prediction of membrane topology (Hartmann et al.) |
| | Center position for calculation: 15 |
| | Charge difference: −3.5 C(−1.0) − N(2.5) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 4.35 |
| Hyd Moment(95): | 3.78 | G content: | 2 |
| D/E content: | 1 | S/T content: | 3 |
| Score: −3.73 | | | |

Gavel: prediction of cleavage sites for mitochondrial preseq

R-2 motif at 39 LRA|EA

NUCDISC: discrimination of nuclear localization signals

pat4: RRRP (4) at 876
pat7: none
bipartite: none
content of basic residues: 9.0%
NLS Score: −0.22

KDEL: ER retention motif in the C-terminus: none
ER Membrane Retention Signals:

XXRR-like motif in the N-terminus: AWLR
KKXX-like motif in the C-terminus: YIKG

SKL: peroxisomal targeting signal in the C-terminus: none
PTS2: 2nd peroxisomal targeting signal: none
VAC: possible vacuolar targeting motif: found

ILPI at 59

RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

Final Results (k = 9/23):

66.7%: endoplasmic reticulum
11.1%: nuclear
11.1%: vesicles of secretory system
11.1%: mitochondrial

>> prediction for CG56258-04 is end (k = 9)

A search of the NOV55a protein against the Geneseq database, a proprietary database that contains sequences published in patents and patent publication, yielded several homologous proteins shown in Table 55D.

TABLE 55D
Geneseq Results for NOV55a

| | | NOV55a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB83246 | Human transporter protein - | 1 . . . 921 | 921/921 (100%) | 0.0 |
| | Homo sapiens, 921 aa. | 1 . . . 921 | 921/921 (100%) | |
| | [WO200233086-A2, 25-APR- | | | |
| | 2002] | | | |
| ABB81913 | Human ion exchanger protein #1 - | 1 . . . 921 | 921/921 (100%) | 0.0 |
| | Homo sapiens, 921 aa. | 1 . . . 921 | 921/921 (100%) | |
| | [WO200259316-A2, 01-AUG- | | | |
| | 2002] | | | |
| ABP74104 | Human TRICH SEQ ID NO 9 - | 1 . . . 921 | 921/921 (100%) | 0.0 |
| | Homo sapiens, 921 aa. | 1 . . . 921 | 921/921 (100%) | |
| | [WO200246415-A2, 13-JUN- | | | |
| | 2002] | | | |
| ABB81915 | Human ion exchanger protein #1 | 1 . . . 921 | 920/921 (99%) | 0.0 |
| | Asp/Gly mutant - Homo sapiens, | 1 . . . 921 | 920/921 (99%) | |
| | 921 aa. [WO200259316-A2, 01- | | | |
| | AUG-2002] | | | |
| ABB81916 | Human ion exchanger protein #1 | 1 . . . 921 | 921/922 (99%) | 0.0 |
| | Ala mutant - Homo sapiens, 922 | 1 . . . 922 | 921/922 (99%) | |
| | aa. [WO200259316-A2, 01- | | | |
| | AUG-2002] | | | |

In a BLAST search of public sequence databases, the NOV55a protein was found to have homology to the proteins shown in the BLASTP data in Table 55E.

TABLE 55E
Public BLASTP Results for NOV55a

| | | NOV55a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IUF0 | Na+/Ca2+ exchanger isoform 3 | 1 . . . 921 | 921/921 (100%) | 0.0 |
| | splice variant 2 - Homo sapiens | 1 . . . 921 | 921/921 (100%) | |
| | (Human), 921 aa. | | | |
| CAD12716 | Sequence 1 from Patent | 1 . . . 921 | 921/927 (99%) | 0.0 |
| | WO0183744 - Homo sapiens | 1 . . . 927 | 921/927 (99%) | |
| | (Human), 927 aa (fragment). | | | |
| Q8NFI7 | Na+/Ca2+ exchanger isoform 3 - | 1 . . . 921 | 921/927 (99%) | 0.0 |
| | Homo sapiens (Human), 927 aa. | 1 . . . 927 | 921/927 (99%) | |
| Q96QG1 | Sodium/calcium exchanger | 1 . . . 921 | 918/924 (99%) | 0.0 |
| | SCL8A3 - Homo sapiens | 1 . . . 924 | 919/924 (99%) | |
| | (Human), 924 aa. | | | |
| P70549 | Sodium/calcium exchanger 3 | 1 . . . 921 | 897/927 (96%) | 0.0 |
| | precursor (Na(+)/Ca(2+)- | 1 . . . 927 | 911/927 (97%) | |
| | exchange protein 3) - Rattus | | | |
| | norvegicus (Rat), 927 aa. | | | |

PFam analysis indicates that the NOV55a protein contains the domains shown in the Table 55F.

TABLE 55F
Domain Analysis of NOV55a

| | | Identities/ | |
|---|---|---|---|
| Pfam | NOV55a | Similarities | |
| Domain | Match Region | for the Matched Region | Expect Value |
| Na_Ca_Ex | 89 . . . 248 | 41/181 (23%) | 1.6e−33 |
| | | 134/181 (74%) | |
| Calx-beta | 386 . . . 485 | 50/106 (47%) | 1.4e−45 |
| | | 88/106 (83%) | |
| Calx-beta | 519 . . . 619 | 51/106 (48%) | 3.5e−46 |
| | | 95/106 (90%) | |
| Na_Ca_Ex | 757 . . . 910 | 44/178 (25%) | 8.7e−31 |
| | | 128/178 (72%) | |

### Example 56

The NOV56 clone was analyzed, and the nucleotide and encoded polypeptide sequences are shown in Table 56A.

TABLE 56A
NOV56 Sequence Analysis

| | | |
|---|---|---|
| NOV56a, CG56262-01 | SEQ ID NO: 871 | 1551 bp |

| | | |
|---|---|---|
| DNA Sequence | ORF Start: ATG at 108 | ORF Stop: TGA at 1512 |

GCGGCCGCGGGAGCTGACCCTGCGGGGTCCCGGGGGGGGAGGGGGAGCCGCGAAGCCCCCACTGACGC
CGCCGCTGCCGGGCCTCCCCTCCCCCCCGGGCGGGCGCCATGCGGGGGAGCCCGGGCGACGCGGAGCG
GCGGCAGCGCTGGGGTCGCCTGTTCGAGGAGCTGGACAGTAACAAGGATGGCCGCGTGGACGTGCACG
AGTTGCGCCAGGGGCTGGCCAGGCTGGGCGGGGGCAACCCAGACCCCGGCGCCCAACAGGGTATCTCC
TCTGAGGGTGATGCTGACCCAGATGGCGGGCTCGACCTGGAGGAATTTTCCCGCTATCTGCAGGAGCG
GGAACAGCGTCTGCTGCTCATGTTTCACAGTCTTGACCGGAACCAGGATGGTCACATTGATGTCTCTG
AGATCCAACAGAGTTTCCGAGCTCTGGGCATTTCCATCTCGCTGGAGCAGGCTGAGAAAATTTTGCAC
AGCATGGACCGAGACGGCACAATGACCATTGACTGGCAAGAATGGCGCGACCACTTCCTGTTGCATTC
GCTGGAAAATGTGGAGGACGTGCTGTATTTCTGGAAGCATTCCACGGTCCTGGACATTGGCGAGTGCC
TGACAGTGCCGGACGAGTTCTCAAAGCAAGAGAAGCTGACGGGCATGTGGTGGAAACAGCTGGTGGCC
GGCGCAGTGGCAGGTGCCGTGTCACGGACAGGCACGGCCCCTCTGGACCGCCTCAAGGTCTTCATTCA
GGTCCATGCCTCAAAGACCAACCGGCTGAACATCCTTGGGGGGCTTCGAAGCATGGTCCTTGAGGGAG
GCATCCGCTGCCTGTGGCGCGGCAATGGTATTAATGTACTCAAGATTGCCCCCGAGTCAGCTATCAAG
TTCATGGCCTATGAACAGGTGAGGAGGGCCATCCTGGGGCAGCAGGAGACACTGCATGTGCAGGAGCG
CTTCGTGGCTGGCTCCCTGGCTGGTGCCACAGCCCAAACCATCATTTACCCTATGGAGGTGCTGAAGA
CGCGGCTGACCTTGCGCCGGACGGGCCAGTATAAGGGGCTGCTGGACTGCGCCAGGCGTATCCTGGAG
AGGGAGGGGCCCCGTGCCTTCTACCGCGGCTACCTCCCCAACGTGCTGGGCATCATCCCCTATGCGGG
CATCGACCTGGCCGTCTACGAGGTCCTGAAGAACTGGTGGCTTCAGCAGTACAGCCACGACTCGGCAG
ACCCAGGCATCCTCGTGCTCCTGGCCTGCGGTACCATATCCAGCACCTGCGGCCAGATAGCCAGTTAC
CCGCTGGCCCTGGTCCGGACCCGCATGCAGGCACAAGCCTCCATCGAGGGTGGCCCCCAGCTGTCCAT
GCTGGGTCTGCTACGTCACATCCTGTCCCAGGAGGGCATGCGGGGCCTCTACCGGGGGATCGCCCCCA
ACTTCATGAAGGTTATTCCAGCTGTGAGCATCTCCTATGTGGTCTACGAGAACATGAAGCAGGCCTTG
GGGGTCACGTCCAGGTGAGGGACCCGGAGCCCGTCCCCCCAATCCCTCACCCCCC

NOV56a, CG56262-01

| | | | |
|---|---|---|---|
| Protein Sequence | SEQ ID NO: 872 | 468 aa | MW at 52387.5kD |

MRGSPGDAERRQRWGRLFEELDSNKDGRVDVHELRQGLARLGGGNPDPGAQQGISSEGDADPDGGLDL
EEFSRYLQEREQRLLLMFHSLDRNQDGHIDVSEIQQSFRALGISISLEQAEKILHSMDRDGTMTIDWQ
EWRDHFLLHSLENVEDVLYFWKHSTVLDIGECLTVPDEFSKQEKLTGMWWKQLVAGAVAGAVSTRGTA
PLDRLKVFIQVHASKTNRLNILGGLRSMVLEGGIRCLWRGNGINVLKIAPESAIKFMAYEQVRRAILG
QQETLHVQERFVAGSLAGATAQTIIYPMEVLKTRLTLRRTGQYKGLLDCARRILEREGPRAFTRGYLP
NVLGIIPYAGIDLAVYEVLKNWWLQQYSHDSADPGILVLLACGTISSTCGQIASYPLALVRTRMQAQA
SIEGGPQLSMLGLLRHILSQEGMRGLYRGIAPNFMKVIPAVSISYVVYENMKQALGVTSR

| | | |
|---|---|---|
| NOV56b, 266120550 | SEQ ID NO: 873 | 1426 bp |

| | | |
|---|---|---|
| DNA Sequence | ORF Start: at 2 | ORF Stop: end of sequence |

CACCGGATCCACCATGCGGGGGAGCCCGGGCGACGCGGAGCGGCGGCAGCGCTGGGGTCGCCTGTTCG
AGGAGCTGGACAGTAACAAGGATGGCCGCGTGGACGTGCACGAGTTGCGCCAGGGGCTGGCCAGGCTG
GGCGGGGGCAACCCAGACCCCGGCGCCCAACAGGGTATCTCCTCTGAGGGTGATGCTGACCCAGATGG
CGGGCTCGACCTGGAGGAATTTTCCCGCTATCTGCAGGAGCGGGAACAGCGTCTGCTGCTCATGTTTC
ACAGTCTTGACCGGAACCAGGATGGTCACATTGATGTCTCTGAGATCCAACAGAGTTTCCGAGCTCTG
GGCATTTCCATCTCGCTGGAGCAGGCTGAGAAAATTTTGCACAGCATGGACCGAGACGGCACAATGAC
CATTGACTGGCAAGAATGGCGCGACCACTTCCTGTTGCATTCGCTGGAAAATGTGGAGGACGTGCTGT
ATTTCTGGAAGCATTCCACGGTCCTGGACATTGGCGAGTGCCTGACAGTGCCGGACGAGTTCTCAAAG
CAAGAGAAGCTGACGGGCATGTGGTGGAAACAGCTGGTGGCCGGCGCAGTGGCAGGTGCCGTGTCACG
GACAGGCACGGCCCCTCTGGACCGCCTCAAGGTCTTCATGCAGGTCCATGCCTCAAAGACCAACCGGC
TGAACATCCTTGGGGGGCTTCGAAGCATGGTCCTTGAGGGAGGCATCCGCTCCCTGTGGCGCGGCAAT
GGTATTAATGTACTCAAGATTGCCCCCGAGTCAGCTATCAAGTTCATGGCCTATGAACAGATCAAGAG
GGCCATCCTGGGGCAGCAGGAGACACTGCATGTGCAGGAGCGCTTCGTGGCTGGCTCCCTGGCTGGTG
CCACAGCCCAAACCATCATTTACCCTATGGAGGTGCTGAAGACGCGGCTGACCTTGCGCCGGACGGGC
CAGTATAAGGGGCTGCTGGACTGCGCCAGGCGTATCCTGGAGAGGGAGGGGCCCCGTGCCTTCTACCG
CGGCTACCTCCCCAACGTGCTGGGCATCATCCCCTATGCGGGCATCGACCTGGCCGTCTACGAGACTC
TGAAGAACTGGTGGCTTCAGCAGTACAGCCACGACTCGGCAGACCCAGGCATCCTCGTGCTCCTGGCC
TGCGGTACCATATCCAGCACCTGCGGCCAGATAGCCAGTTACCCGCTGGCCCTGGTCCGGACCCGCAT
GCAGGCACAAGCCTCCATCGAGGGTGGCCCCCAGCTGTCCATGCTGGGTCTGCTACGTCACATCCTGT
CCCAGGAGGGCATGCGGGGCCTCTACCGGGGGATCGCCCCCAACTTCATGAAGGTTATTCCAGCTGTG
AGCATCTCCTATGTGGTCTACGAGAACATGAAGCAGGCCTTGGGGGTCACGTCCAGGCTCGAGGGC

NOV56b, 266120550

| | | | |
|---|---|---|---|
| Protein Sequence | SEQ ID NO: 874 | 475 aa | MW at 53023.1kD |

TGSTMRGSPGDAERRQRWGRLFEELDSNKDGRVDVHELRQGLARLGGGNPDPGAQQGISSEGDADPDG
GLDLEEFSRYLQEREQRLLLMFHSLDRNQDGHIDVSEIQQSFRALGISISLEQAEKILHSMDRDGTMT
IDWQEWRDHFLLHSLENVEDVLYFWKHSTVLDIGECLTVPDEFSKQEKLTGMWWKQLVAGAVAGAVSR
TGTAPLDRLKVFMQVHASKTNRLNILGGLRSMVLEGGIRSLWRGNGINVLKIAPESAIKFMAYEQIKR
AILGQQETLHVQERFVAGSLAGATAQTIIYPMEVLKTRLTLRRTGQYKGLLDCARRILEREGPRAFYR
GYLPNVLGIIPYAGIDLAVYETLKNWWLQQYSHDSADPGILVLLACGTISSTCGQIASYPLALVRTRM
QAQASIEGGPQLSMLGLLRHILSQEGMRGLYRGIAPNFMKVIPAVSISYVVYENMKQALGVTSRLEG

A ClustalW comparison of the above protein sequences yields the following sequence alignment shown in Table 56B.

TABLE 56B
Comparison of the NOV56 protein sequences.

| | |
|---|---|
| NOV56a | ----MRGSPGDAERRQRWGRLFEELDSNKDGRVDVHELRQGLARLGGGNPDPGAQQGISS |
| NOV56b | TGSTMRGSPGDAERRQRWGRLFEELDSNKDGRVDVHELRQGLARLGGGNPDPGAQQGISS |
| NOV56a | EGDADPDGGLDLEEFSRYLQEREQRLLLMFHSLDRNQDGHIDVSEIQQSFRALGISISLE |
| NOV56b | EGDADPDGGLDLEEFSRYLQEREQRLLLMFHSLDRNQDGHIDVSEIQQSFRALGISISLE |
| NOV56a | QAEKILHSMDRDGTMTIDWQEWRDHFLLHSLENVEDVLYFWKHSTVLDIGECLTVPDEFS |
| NOV56b | QAEKILHSMDRDGTMTIDWQEWRDHFLLHSLENVEDVLYFWKHSTVLDIGECLTVPDEFS |
| NOV56a | KQEKLTGNWWKQLVAGAVAGAVSRTGTAPLDRLKVFIQVHASKTNRLNILGGLRSMVLEG |
| NOV56b | KQEKLTGNWWKQLVAGAVAGAVSRTGTAPLDRLKVFIQVHASKTNRLNILGGLRSMVLEG |
| NOV56a | GIRCLWRGNGINVLKIAPESAIKFMAYEQVRRAILGQQETLHVQERFVAGSLAGATAQTI |
| NOV56b | GIRSLWRGNGINVLKIAPESAIKFMAYEQIKRAILGQQETLHVQERFVAGSLAGATAQTI |
| NOV56a | IYPMEVLKTRLTLRRTGQYKGLLDCARRILEREGPRAFYRGYLPNVLGIIPYAGIDLAVY |
| NOV56b | IYPMEVLKTRLTLRRTGQYKGLLDCARRILEREGPRAFYRGYLPNVLGIIPYAGIDLAVY |
| NOV56a | EVLKNWWLQQYSHDSADPGILVLLACGTISSTCGQIASYPLALVRTRMQAQASIEGGPQL |
| NOV56b | ETLKNWWLQQYSHDSADPGILVLLACGTISSTCGQIASYPLALVRTRMQAQASIEGGPQL |
| NOV56a | SMLGLLRHILSQEGMRGLYRGIAPNFMKVIPAVSISYVVYENMKQALGVTSR--- |
| NOV56b | SMLGLLRHILSQEGMRGLYRGIAPNFMKVIPAVSISYVVYENMKQALGVTSRLEG |
| NOV56a | (SEQ ID NO: 872) |
| NOV56b | (SEQ ID NO: 874) |

Further analysis of the NOV56a protein yielded the following properties shown in Table 56C.

TABLE 56C
Protein Sequence Properties NOV56a

| | |
|---|---|
| SignalP analysis: | No Known Signal Sequence Indicated |
| PSORT II analysis: | |

| | |
|---|---|
| PSG: | a new signal peptide prediction method |
| | N-region: length 11; pos.chg 3; neg.chg 2 |
| | H-region: length 1; peak value −14.40 |
| | PSG score: −18.80 |
| GvH: | von Heijne's method for signal seq. recognition |
| | GvH score (threshold: −2.1): −7.60 |
| | possible cleavage site: between 46 and 47 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 1 |
| | Number of TMS(s) for threshold 0.5: 0 |
| | PERIPHERAL Likelihood = 0.85 (at 342) |
| | ALOM score: −0.90 (number of TMSs: 0) |

MITDISC: discrimination of mitochondrial targeting seq

| | | | |
|---|---|---|---|
| R content: | 1 | Hyd Moment(75): | 10.59 |
| Hyd Moment(95): | 7.77 | G content: | 2 |
| D/E content: | 2 | S/T content: | 1 |
| Score: −5.97 | | | |

Gavel: prediction of cleavage sites for mitochondrial preseq

R-2 motif at 12 MRG|SP

NUCDISC: discrimination of nuclear localization signals

pat4: none
pat7: none
bipartite: none
content of basic residues: 11.3%
NLS Score: −0.47

KDEL: ER retention motif in the C-terminus: none
ER Membrane Retention Signals:

XXRR-like motif in the N-terminus: RGSP

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***

LEEFSRYLQEREQRLLLMFHSL at 68

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

```
52.2%: cytoplasmic
30.4%: nuclear
  8.7%: mitochondrial
  4.3%: vacuolar
  4.3%: vesicles of secretory system
```

>> prediction for CG56262-01 is cyt (k = 23)

A search of the NOV56a protein against the Geneseq database, a proprietary database that contains sequences published in patents and patent publication, yielded several homologous proteins shown in Table 56D.

TABLE 56D
Geneseq Results for NOV56a

| | | NOV56a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE22927 | Human transporter and ion | 1 . . . 468 | 463/468 (98%) | 0.0 |
| | channel (TRICH) 26 - Homo | 1 . . . 468 | 466/468 (98%) | |
| | sapiens, 468 aa. [WO200222684- | | | |
| | A2, 21-MAR-2002] | | | |
| AAU27869 | Human contig polypeptide | 1 . . . 468 | 463/468 (98%) | 0.0 |
| | sequence #22 - Homo sapiens, 509 | 39 . . . 506 | 466/468 (98%) | |
| | aa. [WO200164834-A2, 07-SEP- | | | |
| | 2001] | | | |
| AAU27697 | Human full-length polypeptide | 1 . . . 468 | 463/468 (98%) | 0.0 |
| | sequence #22 - Homo sapiens, 471 | 1 . . . 468 | 466/468 (98%) | |
| | aa. [WO200164834-A2, 07-SEP- | | | |
| | 2001] | | | |
| ABG22637 | Novel human diagnostic protein | 1 . . . 468 | 442/470 (94%) | 0.0 |
| | #22628 - Homo sapiens, 508 aa. | 39 . . . 508 | 450/470 (95%) | |
| | [WO200175067-A2, 11-OCT- | | | |
| | 2001] | | | |
| ABG30434 | Human protein sequence #2 used | 1 . . . 409 | 403/409 (98%) | 0.0 |
| | for determining sequence of | 1 . . . 409 | 406/409 (98%) | |
| | unknown gene - Homo sapiens, | | | |
| | 456 aa. [JP2002176980-A, 25- | | | |
| | JUN-2002] | | | |

In a BLAST search of public sequence databases, the NOV56a protein was found to have homology to the proteins shown in the BLASTP data in Table 56E.

TABLE 56E
Public BLASTP Results for NOV56a

| | | NOV56a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD55563 | Putative calcium binding | 1 . . . 407 | 402/407 (98%) | 0.0 |
| | transporter - Homo sapiens | 1 . . . 407 | 405/407 (98%) | |
| | (Human), 438 aa. | | | |
| Q96NQ4 | Hypothetical protein FLJ30339 - | 85 . . . 468 | 379/384 (98%) | 0.0 |
| | Homo sapiens (Human), 384 aa. | 1 . . . 384 | 382/384 (98%) | |
| CAD20531 | Sequence 1 from Patent | 1 . . . 407 | 374/407 (91%) | 0.0 |
| | WO0174854 - Homo sapiens | 1 . . . 406 | 388/407 (94%) | |
| | (Human), 460 aa (fragment). | | | |
| AAH43834 | Similar to hypothetical protein | 6 . . . 468 | 302/463 (65%) | e−177 |
| | MGC36388 - Xenopus laevis | 53 . . . 514 | 368/463 (79%) | |
| | (African clawed frog), 514 aa. | | | |
| Q8BHG0 | Weakly similar to peroxisomal | 6 . . . 468 | 305/463 (65%) | e−176 |
| | CA-dependent solute carrier - | 41 . . . 502 | 366/463 (78%) | |
| | Mus musculus (Mouse), 502 aa. | | | |

PFam analysis indicates that the NOV56a protein contains the domains shown in the Table 56F.

TABLE 56F
Domain Analysis of NOV56a

| | | Identities/ | |
|---|---|---|---|
| | | Similarities | |
| Pfam | NOV56a | for the Matched | Expect |
| Domain | Match Region | Region | Value |
| efhand | 13 . . . 41 | 8/29 (28%) | 0.00022 |
| | | 26/29 (90%) | |
| efhand | 81 . . . 109 | 8/29 (28%) | 0.012 |
| | | 21/29 (72%) | |
| mito_carr | 184 . . . 276 | 38/124 (31%) | 2.1e−24 |
| | | 78/124 (63%) | |
| mito_carr | 278 . . . 369 | 38/124 (31%) | 3.4e−32 |
| | | 81/124 (65%) | |
| mito_carr | 375 . . . 468 | 31/124 (25%) | 2.4e−23 |
| | | 76/124 (61%) | |

### Example 57

The NOV57 clone was analyzed, and the nucleotide and encoded polypeptide sequences are shown in Table 57A.

TABLE 57A
NOV57 Sequence Analysis

| | | |
|---|---|---|
| NOV57a, CG56315-01 | SEQ ID NO: 875 | 728 bp |

| | | |
|---|---|---|
| DNA Sequence | ORF Start: ATG at 28 | ORF Stop: TGA at 697 |

AGTCTTGCCTTCTTTTGAGCCTAAGTCATGAGTTGGATGTTCCTCAGAGATCTCCTGAGTGGAGTAAA
TAAATACTCCACTGGGACTGGATGGATTTGGCTGGCTGTCGTGTTTGTCTTCCGTTTGCTGGTCTACA
TGGTGGCAGCAGAGCACGTGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATGGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTCTTGGTCATCACCTCATGCTTGTGTATTGTGTTGAATTTC
ATTGAACTGAGTTTTTTGGTTCTCAAGTGCTTTATTAAGTGCTGTCTCCAAAAATATTTAAAAAAACC
TCAAGTCCTCAGTGTGTGAGTGCCACAGCCTCAGATATGTTGAATGTG

NOV57a, CG56315-01

| | | | |
|---|---|---|---|
| Protein Sequence | SEQ ID NO: 876 | 223 aa | MW at 25859.7kD |

MSWMFLRDLLSGVNKYSTGTGWIWLAVVFVFRLLVYMVAAEHVWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CFIKCCLQKYLKKPQVLSV

| | | |
|---|---|---|
| NOV57b, 247679382 | SEQ ID NO: 877 | 700 bp |

| | | |
|---|---|---|
| DNA Sequence | ORF Start: at 2 | ORF Stop: end of sequence |

AGGCTCCGCGGCCGCCCCCTTCACCGGATCCATGAGTTGGATGTTCCTCAGAGATCTCCTGAGTGGAG
TAAATAAATACTCCACTGGGATTGGATGGATTTGGCTGGCTGTCGTGTTTGTCTTCCGTTTGCTGGTC
TACATGGTGGCAGCAGAGCACGTGTGGAAAGATGAGCAGAAAGAGTTTGAGTGCAACAGTAGACAGCC
CGGTTGCAAAAATGTGTGTTTTGATGACTTCTTCCCCATTTCCCAAGTCAGACTTTGGGCCGTACAAC
TGATAATGGTCTCCACACCTTCACTTCTGGTGGTTTTACATGTAGCCTATCATGAGGGTAGAGAGAAA
AGGCACAGAAAGAAACTCTATGTCAGCCCAGGTACAATGGATGGGGGCCTATGGTACGCTTATCTTAT
CAGCCTCATTGTTAAAACTGGTTTTGAAATTGGCTTCCTTGTTTTATTTTATAAGCTATATGATGGCT
TTAGTGTTCCCTACCTTATAAAGTGTGATTTGAAGCCTTGTCCCAACACTGTGGACTGCTTCATCTCC
AAACCCACTGAGAAGACGATCTTCATCCTCTTCTTGGTCATCACCTCATGCTTGCGTATTGTGTTGAA
TTTCATTGAACTGAGTTTTTTGCTCGAGGGCAAGGGTGGGCGCGCCGACCCAGCTTTCTTGTACAAAA
GTTGGCATTTTATAAGAAAA

NOV57b, 247679382

| | | | |
|---|---|---|---|
| Protein Sequence | SEQ ID NO: 878 | 233 aa | MW at 26752.3kD |

GSAAAPFTGSMSWMFLRDLLSGVNKYSTGIGWIWLAVVFVFRLLVYMVAAEHVWKDEQKEFECNSRQP
GCKNVCFDDFFPISQVRLWAVQLIMVSTPSLLVVLHVAYHEGREKRHRKKLYVSPGTMDGGLWYAYLI
SLIVKTGFEIGFLVLFYKLYDGFSVPYLIKCDLKPCPNTVDCFISKPTEKTIFILFLVITSCLRIVLN
FIELSFLLEGKGGRADPAFLYKSWHFIRK

| | | |
|---|---|---|
| NOV57c, 247678321 | SEQ ID NO: 879 | 724 bp |

| | | |
|---|---|---|
| DNA Sequence | ORF Start: at 2 | ORF Stop: end of sequence |

AGGCTCCGCGGCCGCCCCCTTCACCGGATCCATGAGTTGGATGTTCCTCAGAGATCTCCTGAGTGGAG
TAAATAAATACTCCACTGGGATTGGATGGATTTGGCTGGCTGTCGTGTTTGTCTTCCGTTTGCTGGTC
TACATGGTGGCAGCAGAGCACGTGTGGAAAGATGAGCAGAAAGAGTTTGAGTGCAACAGTAGACAGCC
CGGTTGCAAAAATGTGTGTTTTGATGACTTCTTCCCCATTTCCCAAGTCAGACTTTGGGCCTTACAAC
TGATAATGGTCTCCACACCTTCACTTCTGGTGGTTTTACATGTAGCCTATCATGAGGGTAGAGAGAAA
AGGCACAGAAAGAAACTCTATGTCAGCCCAGGTACAATGGATGGGGGCCTATGGTACGCTTATCTTAT
CAGCCTCATTGTTAAAACTGGTTTTGAAATTGGCTTCCTTGTTTTATTTTATAAGCTATATGATGGCT
TTAGTGTTCCCTACCTTATAAAGTGTGATTTGAAGCCTTGTCCCAACACTGTGGACTGCTTCATCTCC
AAACCCACTGAGAAGACGATCTTCATCCTCTTCTTGGTCATCACCTCATGCTTGCGTATTGTGTTGAA
TTCCATTGAACTGAGTTTTTTGGTTCTCAAGTGCCTTATTAAGTGCTGTCTCCAAAAATATTTAAAAA
AACCTCAAGTCCTCAGTGTGCTCGAGGGCAAGGGTGGGCGCGCC

NOV57c, 247678321

| | | | |
|---|---|---|---|
| Protein Sequence | SEQ ID NO: 880 | 241 aa | MW at 27446.4kD |

GSAAAPFTGSMSWMFLRDLLSGVMKYSTGIGWIWLAVVFVFRLLVYMVAAEHVWKDEQKEFEGNSRQP
GCKNVCFDDFFPISQVRLWALQLIMVSTPSLLVVLHVAYHEGREKRHRKKLYVSPGTMDGGLWYAYLI
SLIVKTGFEIGFLVLFYKLYDGFSVPYLIKCDLKPCPNTVDCFISKPTEKTIFILFLVITSCLRIVLN
SIELSFLVLKCLIKCCLQKYLKKPQVLSVLEGKGGRA

| | | |
|---|---|---|
| NOV57d, 247679418 | SEQ ID NO: 881 | 538 bp |

| | | |
|---|---|---|
| DNA Sequence | ORF Start: at 2 | ORF Stop: end of sequence |

AGGCTCCGCGGCCGCCCCCTTCACCGGATCCGAGCACGTGTGGAAAGATGAGCAGAAAGAGTTTGAGT
GCAACAGTAGACAGCCCGGTTGCAAAAATGTGTGTTTTGATGACTTCTTCCCCATTTCCCAAGTCAGA
CTTTGGGCCTTACAACTGATAATGGTCTCCACACCTTCACTTCTGGTGGTTTTACATGTAGCCTATCA
TGAGGGTAGAGAGAAAAGGCACAGAAAGAAACTCTATGTCAGCCCAGGTACAATGGATGGGGGCCTAT
GGTACGCTTATCTTATCAGCCTCATTGTTAAAACTGGTTTTGAAATTGGCTTCCTTGTTTTATTTTA2
AAGCTATATGATGGCTTTAGTGTTCCCTACCTTATAAAGTGTGATTTGAAGCCTTGTCCCAACACTGT
GGACTGCTTCATCTCCAAACCCACTGAGAAGACGATCTTCATCCTCTTCTTGGTCATCACCTCATGCT
TGCGTATTGTGTTGAATTTCATTGAACTGAGTTTTTTGCTCGAGGGCAAGGGTGGGCGCGCC

NOV57d, 247679418

| | | | |
|---|---|---|---|
| Protein Sequence | SEQ ID NO: 882 | 179 aa | MW at 20339.7kD |

GSAAAPFTGSEHVWKDEQKEFECNSRQPGCIQNCFDDFFPISQVRLWALQLIMVSTPSLLVVLHVAYH
EGREKRHRKKLYVSPGTMDGGLWYAYLISLIVKTGFEIGFLVLFYKLYDGFSVPYLIKCDLKPCPNTV
DCFISKPTEKTIFILFLVITSCLRIVLNFIELSFLLEGKGGRA

| | | |
|---|---|---|
| NOV57e, 247679395 | SEQ ID NO: 883 | 658 bp |

| | | |
|---|---|---|
| DNA Sequence | ORF Start: at 2 | ORF Stop: end of sequence |

AGGCTCCGCGGCCGCCCCCTTCACCGGATCCATGAGTTGGATGTTCCTCAGAGATCTCCTGAGTGGAG
TAAATAAATACTCCACTGGGATTGGATGGATTTGGCTGGCTGTCGTGTTTGTCTTCCGTTTGCTGGTC
TACATGGTGGCAGCAGAGCACGTGTGGAAAGATGAGCAGAAAGAGTTTGAGTGCAACAGTAGACAGCC
CGGTTGCAAAAATGTGTGTTTTGATGACTTCTTCCCCATTTCCCAAGTCAGACTTTGGGCCTTACAAC
TGATAATGGTCTCCACACCTTCACTTCTGGTGGTTTTACATGTAGCCTATCATGAGGGTAGAGAGAAA
AGGCACAGAAAGAAACTCTATGTCAGCCCAGGTACAATGGATGGGGGCCTATGGTACGCTTATCTTAT
CAGCCTCATTGTTAAAACTGGTTTTGAAATTGGCTTCCTTGTTTTATTTTATAAGCTATATGATGGCT
TTAGTGTTCCCTACCTTATAAAGTGTGATTTGAAGCCTTGTCCCAACACTGTGGACTGCTTCATCTCC
AAACCCACTGAGAAGACGATCTTCATCCTCTTCTTGGTCATCACCTCATGCTTGCGTATTGTGTTGAA
TTTCATTGAACTGAGTTTTTTGCTCGAGGGCAAGGGTGGGCGCGCC

NOV57e, 247679395

| | | | |
|---|---|---|---|
| Protein Sequence | SEQ ID NO: 884 | 219 aa | MW at 24976.2kD |

GSAAAPFTGSMSWMFLRDLLSGVNKYSTGIGWIWLAVVFVFRLLVYMVAAEHVWKDEQKEFECNSRQP
GCKNVCFDDFFPISQVRLWALQLIMVSTPSLLVVLHVAYHEGREKRHRKKLYVSPGTMDGGLWYAYLI
SLIVKTGFEIGFLVLFYKLYDGFSVPYLIKCDLKPCPNTVDCFISKPTEKTIFILFLVITSCLRIVLN
FIELSFLLEGKGGRA

| | | |
|---|---|---|
| NOV57f, 247679328 | SEQ ID NO: 885 | 604 bp |

| | | |
|---|---|---|
| DNA Sequence | ORF Start: at 2 | ORF Stop: end of sequence |

AGGCTCCGCGGCCGCCCCCTTCACCGGATCCGAGCACGTGTGGAAAGATGAGCAGAAAGAGTTTGAGT
GCAACAGTAGACAGCCCGGTTGCAAAAATGTGTGTTTTGATGACTTCTTCCCCATTTCCCAAGTCAGA
CTTTGGGCCTTACAACTGATAATGGTCTCCACACCTTCACTTCTGGTGGTTTTACATGTAGCCTATCA
TGAGGGTAGAGAGAAAAGGCACAGAAAGAAACTCTATGTCAGCCCAGGTACAATGGATGGGGGCCTAT
GGTACGCTTATCTTATCAGCCTCATTGTTAAAACTGGTTTTGAAATTGGCTTCCTTGTTTTATTTTAT
AAGCTATATGATGGCTTTAGTGTTCCCTACCTTATAAAGTGTGATTTGAAGCCTTGTCCCAACACTGT
GGACTGCTTCATCTCCAAACCCACTGAGAAGACGATCTTCATCCTCTTCTTGGTCATCACCTCATGCT
TGCGTATTGTGTTGAATTCCATTGAACTGAGTTTTTTGGTTCTCAAGTGCCTTATTAAGTGCTGTCTC
CAAAAATATTTAAAAAAACCTCAAGTCCTCAGTGTGCTCGAGGGCAAGGGTGGGCGCGCC

NOV57f, 247679328

| | | | |
|---|---|---|---|
| Protein Sequence | SEQ ID NO: 886 | 201 aa | MW at 22809.8kD |

GSAAAPFTGSEHVWKDEQKEFECNSRQPGCKNVCFDDFFPISQVRLWALQLIMVSTPSLLVVLHVAYH
EGREKRHRKKLYVSPGTMDGGLWYAYLISLIVKTGFEIGFLVLFYKLYDGFSVPYLIKCDLKPCPNTV
DCFISKPTEKTIFILFLVITSCLRIVLNSIELSFLVLKCLIKCCLQKYLKKPQVLSVLEGKGGHA

| | | |
|---|---|---|
| NOV57g, CG56315-02 | SEQ ID NO: 887 | 727 bp |

| | | |
|---|---|---|
| DNA Sequence | ORF Start: ATG at 27 | ORF Stop: TGA at 696 |

AGTCTTGCTTCTTTTGAGCCTAAGTCATGAGTTGGATGTTCCTCAGAGATCTCCTGAGTGGAGTAAAT
AAATACTCCACTGGGATTGGATGGATTTGGCTGGCTGTCGTGTTTGTCTTCCGTTTGCTGGTCTACAT
GGTGGCAGCAGAGCACGTGTGGAAAGATGAGCAGAAAGAGTTTGAGTGCAACAGTAGACAGCCCGGTT
GCAAAAATGTGTGTTTTGATGACTTCTTCCCCATTTCCCAAGTCAGACTTTGGGCCTTACAACTGATA
ATGGTCTCCACACCTTCACTTCTGGTGGTTTTACATGTAGCCTATCATGAGGGTAGAGAGAAAAGGCA
CAGAAAGAAACTCTATGTCAGCCCAGGTACAATGGATGGGGGCCTATGGTACGCTTATCTTATCAGCC
TCATTGTTAAAACTGGTTTTGAAATTGGCTTCCTTGTTTTATTTTATAAGCTATATGATGGCTTTAGT
GTTCCCTACCTTATAAAGTGTGATTTGAAGCCTTGTCCCAACACTGTGGACTGCTTCATCTCCAAACC
CACTGAGAAGACGATCTTCATCCTCTTCTTGGTCATCACCTCATGCTTGTGTATTGTGTTGAATTTCA
TTGAACTGAGTTTTTTGGTTCTCAAGTGCTTTATTAAGTGCTGTCTCCAAAAATATTTAAAAAAACCT
CAAGTCCTCAGTGTGTGAGTGCCACAGCCTCAGATATGTTGAATGTG

NOV57g, CG56315-02

| | | | |
|---|---|---|---|
| Protein Sequence | SEQ ID NO: 888 | 223 aa | MW at 25871.7kD |

MSWMFLRDLLSGVNKYSTGIGWIWLAVVFVFRLLVYMVAAEHVWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CFIKCCLQKYLKKPQVLSV

| | | |
|---|---|---|
| NOV57h, CG56315-03 | SEQ ID NO: 889 | 24 bp |

| | | |
|---|---|---|
| DNA Sequence | ORF Start: at 1 | ORF Stop: end of sequence |

TTTGAGCAAAACAGAAGACAGCCC

NOV57h, CG56315-03

| | | | |
|---|---|---|---|
| Protein Sequence | SEQ ID NO: 890 | 8 aa | MW at 1074.2kD |

FEQNRRQP

| | | |
|---|---|---|
| NOV57i, CG56315-04 | SEQ ID NO: 891 | 24 bp |

| | | |
|---|---|---|
| DNA Sequence | ORF Start: at 1 | ORF Stop: end of sequence |

TTTGAGTGCAACAGGAGACAGCCC

NOV57i, CG56315-04

| | | | |
|---|---|---|---|
| Protein Sequence | SEQ ID NO: 892 | 8 aa | MW at 1049.2kD |

FECNRRQP

| | | |
|---|---|---|
| NOV57j, CG56315-05 | SEQ ID NO: 893 | 24 bp |

| | | |
|---|---|---|
| DNA Sequence | ORF Start: at 1 | ORF Stop: end of sequence |

TTTGAGCAAAACAGTAGACAGCCC

NOV57j, CG56315-05

| | | | |
|---|---|---|---|
| Protein Sequence | SEQ ID NO: 894 | 8 aa | MW at 1005.1kD |

FEQNSRQP

| | | |
|---|---|---|
| NOV57k, CG56315-06 | SEQ ID NO: 895 | 24 bp |

| | | |
|---|---|---|
| DNA Sequence | ORF Start: at 1 | ORF Stop: end of sequence |

TTTGAGTGCAACAGTAGACAGCCC

NOV57k, CG56315-06

| | | | |
|---|---|---|---|
| Protein Sequence | SEQ ID NO: 896 | 8 aa | MW at 980.1kD |

FECNSRQP

| | | |
|---|---|---|
| NOV57l, CG56315-07 | SEQ ID NO: 897 | 24 bp |

| | | |
|---|---|---|
| DNA Sequence | ORF Start: at 1 | ORF Stop: end of sequence |

TTTGAGCAAAACAGTAGACAGGCC

NOV57l, CG56315-07

| | | | |
|---|---|---|---|
| Protein Sequence | SEQ ID NO: 898 | 8 aa | MW at 979.0kD |

FEQNSRQA

| | | |
|---|---|---|
| NOV57m, CG56315-08 | SEQ ID NO: 899 | 24 bp |

| | | |
|---|---|---|
| DNA Sequence | ORF Start: at 1 | ORF Stop: end of sequence |

TTTGAGTGCAACAGTAGACAGGCC

NOV57m, CG56315-08

| | | | |
|---|---|---|---|
| Protein Sequence | SEQ ID NO: 900 | 8 aa | MW at 954.0kD |

FECNSRQA

| | |
|---|---|
| SEQ ID NO: 901 | 728 bp |

| | | |
|---|---|---|
| NOV57n, SNP13381650 of | ORF Start: ATG at 28 | ORF Stop: TGA at 697 |

| | | |
|---|---|---|
| CG56315-01, DNA Sequence | SNP Pos: 86 | SNP Change: C to T |

AGTCTTGCCTTCTTTTGAGCCTAAGTCATGAGTTGGATGTTCCTCAGAGATCTCCTGAGTGGAGTAAA
TAAATACTCCACTGGGATTGGATGGATTTGGCTGGCTGTCGTGTTTGTCTTCCGTTTGCTGGTCTACA
TGGTGGCAGCAGAGCACGTGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATGGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTcTTGGTCATCACCTCATGCTTGTGTATTGTGTTGAATTTC
ATTGAACTGAGTTTTTTGGTTCTCAAGTGCTTTATTAAGTGCTGTCTCCAAAAATATTTAAAAAAACC
TCAAGTCCTCAGTGTGTGAGTGCCACAGCCTCAGATATGTTGAATGTG

| | | |
|---|---|---|
| NOV57n, SNP13381650 of | SEQ ID NO: 902 | MW at 25871.7kD |

| | | | |
|---|---|---|---|
| CG56315-01, Protein Sequence | SNP Pos: 20 | 223 aa | SNP Change: Thr to Ile |

MSWMFLRDLLSGVNKYSTGIGWIWLAVVFVFRLLVYMVAAEHVWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CFIKCCLQKYLKKPQVLSV

| | |
|---|---|
| SEQ ID NO: 903 | 728 bp |

| | | |
|---|---|---|
| NOV57o, SNP13381651 of | ORF Start: ATG at 28 | ORF Stop: TGA at 697 |

| | | |
|---|---|---|
| CG56315-01, DNA Sequence | SNP Pos: 154 | SNP Change: G to A |

AGTCTTGCCTTCTTTTGAGCCTAAGTCATGAGTTGGATGTTCCTCAGAGATCTCCTGAGTGGAGTAAA
TAAATACTCCACTGGGACTGGATGGATTTGGCTGGCTGTCGTGTTTGTCTTCCGTTTGCTGGTCTACA
TGGTGGCAGCAGAGCACATGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATGGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTCTTGGTCATCACCTCATGCTTGTGTATTGTGTTGAATTTC
ATTGAACTGAGTTTTTTGGTTCTCAAGTGCTTTATTAAGTGCTGTCTCCAAAAATATTTAAAAAAACC
TCAAGTCCTCAGTGTGTGAGTGCCACAGCCTCAGATATGTTGAATGTG

| | | |
|---|---|---|
| NOV57o, SNP13381651 of | SEQ ID NO: 904 | MW at 25891.7kD |

| | | | |
|---|---|---|---|
| CG56315-01, Protein Sequence | SNP Pos: 43 | 223 aa | SNP Change: Val to Met |

MSWMFLRDLLSGVNKYSTGTGWIWLAVVFVFRLLVYMVAAEHMWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CFIKCCLQKYLKKPQVLSV

| | |
|---|---|
| SEQ ID NO: 905 | 728 bp |

| | | |
|---|---|---|
| NOV57p, SNP13381652 of | ORF Start: ATG at 28 | ORF Stop: TGA at 697 |

| | | |
|---|---|---|
| CG56315-01, DNA Sequence | SNP Pos: 276 | SNP Change: G to A |

AGTCTTGCCTTCTTTTGAGCCTAAGTCATGAGTTGGATGTTCCTCAGAGATCTCCTGAGTGGAGTAAA
TAAATACTCCACTGGGACTGGATGGATTTGGCTGGCTGTCGTGTTTGTCTTCCGTTTGCTGGTCTACA
TGGTGGCAGCAGAGCACGTGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATAGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTCTTGGTCATCACcTCATGCTTGTGTATTGTGTTGAATTTC
ATTGAACTGAGTTTTTTGGTTCTCAAGTGCTTTATTAAGTGCTGTCTCCAAAAATATTTAAAAAAACC
TCAAGTCCTCAGTGTGTGAGTGCCACAGCCTCAGATATGTTGAATGTG

| | | |
|---|---|---|
| NOV57p, SNP13381652 of | SEQ ID NO: 906 | MW at 25841.6kD |

| | | | |
|---|---|---|---|
| CG56315-01, Protein Sequence | SNP Pos: 83 | 223 aa | SNP Change: Met to Ile |

MSWMFLRDLLSGVNKYSTGTGWIWLAVVFVFRLLVYMVAAEHMWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CFIKCCLQKYLKKPQVLSV

| | |
|---|---|
| SEQ ID NO: 907 | 728 bp |

| | | |
|---|---|---|
| NOV57q, SNP13381653 of | ORF Start: ATG at 28 | ORF Stop: TGA at 697 |

| | | |
|---|---|---|
| CG56315-01, DNA Sequence | SNP Pos: 557 | SNP Change: C to T |

AGTCTTGCCTTCTTTTGAGCCTAAGTCATGAGTTGGATGTTCCTCAGAGATCTCCTGAGTGGAGTAAA
TAAATACTCCACTGGGACTGGATGGATTTGGCTGGCTGTCGTGTTTGTCTTCCGTTTGCTGGTCTACA
TGGTGGCAGCAGAGCACGTGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATAGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTCTTGGTCATCACcTCATGCTTGTGTATTGTGTTGAATTTC
ATTGAACTGAGTTTTTTGGTTCTCAAGTGCTTTATTAAGTGCTGTCTCCAAAAATATTTAAAAAAACC
TCAAGTCCTCAGTGTGTGAGTGCCACAGCCTCAGATATGTTGAATGTG

| | | |
|---|---|---|
| NOV57q, SNP13381653 of | SEQ ID NO: 908 | MW at 25889.8kD |

| | | | |
|---|---|---|---|
| CG56315-01, Protein Sequence | SNP Pos: 177 | 223 aa | SNP Change: Thr to Met |

MSWMFLRDLLSGVNKYSTGTGWIWLAVVFVFRLLVYMVAAEHMWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKMIFILFLVITSCLCIVLNFIELSFLVLK
CFIKCCLQKYLKKPQVLSV

| | |
|---|---|
| SEQ ID NO: 909 | 728 bp |

| | | |
|---|---|---|
| NOV57r, SNP13381654 of | ORF Start: ATG at 28 | ORF Stop: TGA at 697 |

| | | |
|---|---|---|
| CG56315-01, DNA Sequence | SNP Pos: 643 | SNP Change: T to C |

AGTCTTGCCTTCTTTTGAGCCTAAGTCATGAGTTGGATGTTCCTCAGAGATCTCCTGAGTGGAGTAAA
TAAATACTCCACTGGGACTGGATGGATTTGGCTGGCTGTCGTGTTTGTCTTCCGTTTGCTGGTCTACA
TGGTGGCAGCAGAGCACGTGTGGAAAGATGAGCAGAAAGAGTTTGAGTGCAACAGTAGACAGCCCGGT
TGCAAAAATGTGTGTTTTGATGACTTCTTCCCCATTTCCCAAGTCAGACTTTGGGCCTTACAACTGAT
AATAGTCTCCACACCTTCACTTCTGGTGGTTTTACATGTAGCCTATCATGAGGGTAGAGAGAAAAGGC
ACAGAAAGAAACTCTATGTCAGCCCAGGTACAATGGATGGGGGCCTATGGTACGCTTATCTTATCAGC
CTCATTGTTAAAACTGGTTTTGAAATTGGCTTCCTTGTTTTATTTTATAAGCTATATGATGGCTTTAG
TGTTCCCTACCTTATAAAGTGTGATTTGAAGCCTTGTCCCAACACTGTGGACTGCTTCATCTCCAAAC
CCACTGAGAAGACGATCTTCATCCTCTTCTTGGTCATCACCTCATGCTTGTGTATTGTGTTGAATTTC
ATTGAACTGAGTTTTTTGGTTCTCAAGTGCCTTATTAAGTGCTGTCTCCAAAAATATTTAAAAAAACC
TCAAGTCCTCAGTGTGTGAGTGCCACAGCCTCAGATATGTTGAATGTG

| | | |
|---|---|---|
| NOV57r, SNP13381654 of | SEQ ID NO: 910 | MW at 25825.6kD |

| | | | |
|---|---|---|---|
| CG56315-01, Protein Sequence | SNP Pos: 206 | 223 aa | SNP Change: Phe to Leu |

MSWMFLRDLLSGVNKYSTGTGWIWLAVVFVFRLLVYMVAAEHMWKDEQKEFECNSRQPGCKNVCFDDF
FPISQVRLWALQLIMVSTPSLLVVLHVAYHEGREKRHRKKLYVSPGTMDGGLWYAYLISLIVKTGFEI
GFLVLFYKLYDGFSVPYLIKCDLKPCPNTVDCFISKPTEKTIFILFLVITSCLCIVLNFIELSFLVLK
CLIKCCLQKYLKKPQVLSV

A ClustalW comparison of the above protein sequences yields the following sequence alignment shown in Table 57B.

TABLE 57B
Comparison of the NOV57 protein sequences.

| | |
|---|---|
| NOV57a | ----------MSWMFLRDLLGVNKYSTGTGWIWLAVVFVFRLLVYMVAAEHVWKDEQKE |
| NOV57b | GSAAAPFTGSMSWMFLRDLLGVNKYSTGTGWIWLAVVFVFRLLVYMVAAEHVWKDEQKE |
| NOV57c | GSAAAPFTGSMSWMFLRDLLGVNKYSTGTGWIWLAVVFVFRLLVYMVAAEHVWKDEQKE |
| NOV57d | GSAAAPFTG---------------------------------------SEHVWKDEQKE |
| NOV57e | GSAAAPFTGSMSWMFLRDLLGVNKYSTGTGWIWLAVVFVFRLLVYMVAAEHVWKDEQKE |
| NOV57f | GSAAAPFTG---------------------------------------SEHVWKDEQKE |
| NOV57g | ----------MSWMFLRDLLGVNKYSTGTGWIWLAVVFVFRLLVYMVAAEHVWKDEQKE |
| NOV57h | ------------------------------------------------------------ |
| NOV57i | ------------------------------------------------------------ |
| NOV57j | ------------------------------------------------------------ |
| NOV57k | ------------------------------------------------------------ |
| NOV57l | ------------------------------------------------------------ |
| NOV57m | ------------------------------------------------------------ |
| NOV57a | FECNSRQPGCKNVCFDDFFPISQVRLWALQLIMVSTPSLLVVLHVAYHEGREKRHRKKLY |
| NOV57b | FECNSRQPGCKNVCFDDFFPISQVRLWALQLIMVSTPSLLVVLHVAYHEGREKRHRKKLY |
| NOV57c | FECNSRQPGCKNVCFDDFFPISQVRLWALQLIMVSTPSLLVVLHVAYHEGREKRHRKKLY |
| NOV57d | FECNSRQPGCKNVCFDDFFPISQVRLWALQLIMVSTPSLLVVLHVAYHEGREKRHRKKLY |
| NOV57e | FECNSRQPGCKNVCFDDFFPISQVRLWALQLIMVSTPSLLVVLHVAYHEGREKRHRKKLY |
| NOV57f | FECNSRQPGCKNVCFDDFFPISQVRLWALQLIMVSTPSLLVVLHVAYHEGREKRHRKKLY |
| NOV57g | FECNSRQPGCKNVCFDDFFPISQVRLWALQLIMVSTPSLLVVLHVAYHEGREKRHRKKLY |
| NOV57h | FEQNRRQP---------------------------------------------------- |
| NOV57i | FECNRRQP---------------------------------------------------- |
| NOV57j | FEQNSRQP---------------------------------------------------- |
| NOV57k | FECNSRQP---------------------------------------------------- |
| NOV57l | FEQNSRQA---------------------------------------------------- |
| NOV57m | FECNSRQA---------------------------------------------------- |
| NOV57a | VSPGTMDGGLWYAYLISLIVKTGFEIGFLVLFYKLYDGFSVPYLIKCDLKPCPNTVDCFI |
| NOV57b | VSPGTMDGGLWYAYLISLIVKTGFEIGFLVLFYKLYDGFSVPYLIKCDLKPCPNTVDCFI |
| NOV57c | VSPGTMDGGLWYAYLISLIVKTGFEIGFLVLFYKLYDGFSVPYLIKCDLKPCPNTVDCFI |
| NOV57d | VSPGTMDGGLWYAYLISLIVKTGFEIGFLVLFYKLYDGFSVPYLIKCDLKPCPNTVDCFI |
| NOV57e | VSPGTMDGGLWYAYLISLIVKTGFEIGFLVLFYKLYDGFSVPYLIKCDLKPCPNTVDCFI |
| NOV57f | VSPGTMDGGLWYAYLISLIVKTGFEIGFLVLFYKLYDGFSVPYLIKCDLKPCPNTVDCFI |
| NOV57g | VSPGTMDGGLWYAYLISLIVKTGFEIGFLVLFYKLYDGFSVPYLIKCDLKPCPNTVDCFI |
| NOV57h | ------------------------------------------------------------ |
| NOV57i | ------------------------------------------------------------ |
| NOV57j | ------------------------------------------------------------ |
| NOV57k | ------------------------------------------------------------ |
| NOV57l | ------------------------------------------------------------ |
| NOV57m | ------------------------------------------------------------ |
| NOV57a | SKPTEKTIFILFLVITSCLCIVLNFIELSFLVLKCFIKCCLQKYLKKPQVLSV------- |
| NOV57b | SKPTEKTIFILFLVITSCLRIVLNFIELSFLLEGKGGRADPAFLYKSWHFIRK------- |
| NOV57c | SKPTEKTIFILFLVITSCLRIVLNSIELSFLVLKCLIKCCLQKYLKKPQVLSVLEGKGGR |
| NOV57d | SKPTEKTIFILFLVITSCLRIVLNFIELSFLLEGKGGRA--------------------- |
| NOV57e | SKPTEKTIFILFLVITSCLRIVLNFIELSFLLEGKGGRA--------------------- |
| NOV57f | SKPTEKTIFILFLVITSCLRIVLNSIELSFLVLKCLIKCCLQKYLKKPQVLSVLEGKGGR |
| NOV57g | SKPTEKTIFILFLVITSCLCIVLNFIELSFLVLKCFIKCCLQKYLKKPQVLSV------- |
| NOV57h | ------------------------------------------------------------ |
| NOV57i | ------------------------------------------------------------ |
| NOV57j | ------------------------------------------------------------ |
| NOV57k | ------------------------------------------------------------ |
| NOV57l | ------------------------------------------------------------ |
| NOV57m | ------------------------------------------------------------ |
| NOV57a | - |
| NOV57b | - |
| NOV57c | A |
| NOV57d | - |
| NOV57e | - |
| NOV57f | A |
| NOV57g | - |
| NOV57h | - |
| NOV57i | - |
| NOV57j | - |
| NOV57k | - |
| NOV57l | - |
| NOV57m | - |
| NOV57a | (SEQ ID NO: 876) |
| NOV57b | (SEQ ID NO: 878) |
| NOV57c | (SEQ ID NO: 880) |
| NOV57d | (SEQ ID NO: 882) |
| NOV57e | (SEQ ID NO: 884) |
| NOV57f | (SEQ ID NO: 886) |
| NOV57g | (SEQ ID NO: 888) |
| NOV57h | (SEQ ID NO: 890) |
| NOV57i | (SEQ ID NO: 892) |
| NOV57j | (SEQ ID NO: 894) |
| NOV57k | (SEQ ID NO: 896) |
| NOV57l | (SEQ ID NO: 898) |
| NOV57m | (SEQ ID NO: 900) |

Further analysis of the NOV57a protein yielded the following properties shown in Table 57C.

TABLE 57C
Protein Sequence Properties NOV57a

| | |
|---|---|
| SignalP analysis: | Cleavage site between residues 41 and 42 |
| PSORT II analysis: | |

| | |
|---|---|
| PSG: | a new signal peptide prediction method |
| | N-region: length 8; pos.chg 1; neg.chg 1 |
| | H-region: length 6; peak value −2.35 |
| | PSG score: −6.75 |
| GvH: | von Heijne's method for signal seq. recognition |
| | GvH score (threshold: −2.1): −4.71 |
| | possible cleavage site: between 40 and 41 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 5 | | |
| | INTEGRAL | Likelihood = −7.64 | Transmembrane 23-39 |
| | INTEGRAL | Likelihood = −2.60 | Transmembrane 81-97 |
| | INTEGRAL | Likelihood = −3.24 | Transmembrane 125-141 |
| | INTEGRAL | Likelihood = −12.26 | Transmembrane 177-193 |
| | INTEGRAL | Likelihood = −2.81 | Transmembrane 195-211 |

| | |
|---|---|
| PERIPHERAL | Likelihood = 6.36 (at 63) |

| | ALOM score: −12.26 (number of TMSs: 5) |
|---|---|
| MTOP: | Prediction of membrane topology (Hartmann et al.) |
| | Center position for calculation: 30 |
| | Charge difference: −1.5 C(0.5) − N(2.0) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 11.62 |
| Hyd Moment(95): | 9.22 | G content: | 3 |
| D/E content: | 2 | S/T content: | 5 |
| Score: −4.01 | | | |

Gavel: prediction of cleavage sites for mitochondrial preseq

R-2 motif at 42 FRL|LV

NUCDISC: discrimination of nuclear localization signals

pat4: KRHR (3) at 103
pat4: RHRK (3) at 104
pat4: HRKK (3) at 105
pat7: none
bipartite: none
content of basic residues: 11.2%
NLS Score: 0.09

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
33.3%: mitochondrial
11.1%: nuclear

>> prediction for CG56315-01 is end (k = 9)

A search of the NOV57a protein against the Geneseq database, a proprietary database that contains sequences published in patents and patent publication, yielded several homologous proteins shown in Table 57D.

TABLE 57D
Geneseq Results for NOV57a

| | | NOV57a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE26418 | Human transmembrane protein | 1 . . . 223 | 221/223 (99%) | e−131 |
| | (TMP)-4 protein - Homo sapiens, | 1 . . . 223 | 221/223 (99%) | |
| | 223 aa. [WO200234783-A2, 02- | | | |
| | MAY-2002] | | | |
| ABU56673 | Lung cancer-associated | 1 . . . 209 | 123/216 (56%) | 6e−73 |
| | polypeptide #266 - Unidentified, | 1 . . . 216 | 161/216 (73%) | |
| | 273 aa. [WO200286443-A2, 31- | | | |
| | OCT-2002] | | | |
| ABU56449 | Lung cancer-associated | 1 . . . 209 | 123/216 (56%) | 6e−73 |
| | polypeptide #42 - Unidentified, | 1 . . . 216 | 161/216 (73%) | |
| | 273 aa. [WO200286443-A2, 31- | | | |
| | OCT-2002] | | | |
| AAY36192 | Human secreted protein #64 - | 1 . . . 209 | 123/216 (56%) | 6e−73 |
| | Homo sapiens, 273 aa. | 1 . . . 216 | 161/216 (73%) | |
| | [WO9925825-A2, 27-MAY- | | | |
| | 1999] | | | |
| AAY36145 | Human secreted protein #17 - | 1 . . . 209 | 123/216 (56%) | 6e−73 |
| | Homo sapiens, 273 aa. | 1 . . . 216 | 161/216 (73%) | |
| | [WO9925825-A2, 27-MAY- | | | |
| | 1999] | | | |

In a BLAST search of public sequence databases, the NOV57a protein was found to have homology to the proteins shown in the BLASTP data in Table 57E.

TABLE 57E
Public BLASTP Results for NOV57a

| | | NOV57a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96KP0 | Connexin25 - Homo sapiens | 1 . . . 223 | 222/223 (99%) | e−131 |
| | (Human), 223 aa. | 1 . . . 223 | 223/223 (99%) | |
| Q91XA4 | Similar to gap junction membrane | 1 . . . 209 | 123/214 (57%) | 6e−73 |
| | channel protein beta 5 (Gap | 1 . . . 214 | 166/214 (77%) | |
| | junction protein) (Connexin) - | | | |
| | Mus musculus (Mouse), 271 aa. | | | |
| Q02739 | Gap junction beta-5 protein | 1 . . . 209 | 123/214 (57%) | 6e−73 |
| | (Connexin 31.1) (Cx31.1) - Mus | 1 . . . 214 | 166/214 (77%) | |
| | musculus (Mouse), 271 aa. | | | |
| O95377 | Gap junction beta-5 protein | 1 . . . 209 | 123/216 (56%) | 2e−72 |
| | (Connexin 31.1) (Cx31.1) - Homo | 1 . . . 216 | 161/216 (73%) | |
| | sapiens (Human), 273 aa. | | | |
| P28232 | Gap junction beta-5 protein | 1 . . . 213 | 122/218 (55%) | 4e−72 |
| | (Connexin 31.1) (Cx31.1) - Rattus | 1 . . . 218 | 167/218 (75%) | |
| | norvegicus (Rat), 271 aa. | | | |

PFam analysis indicates that the NOV57a protein contains the domains shown in the Table 57F.

TABLE 57F
Domain Analysis of NOV57a

| | | Identities/ | |
|---|---|---|---|
| | | Similarities | |
| Pfam | NOV57a | for the Matched | Expect |
| Domain | Match Region | Region | Value |
| connexin | 2 . . . 201 | 109/247 (44%) | 2.2e−111 |
| | | 166/247 (67%) | |

### Example 58

The NOV58 clone was analyzed, and the nucleotide and encoded polypeptide sequences are shown in Table 58A.

TABLE 58A
NOV58 Sequence Analysis

| | | |
|---|---|---|
| NOV58a, CG56398-01 | SEQ ID NO: 911 | 2105 bp |

| | | |
|---|---|---|
| DNA Sequence | ORF Start: ATG at 31 | ORF Stop: TAG at 2056 |

CCTCAGGATCCAGAGGTCTCGTTCAGGACCATGGAGAGCGGCACCAGCAGCCCTCAGCCTCCACAGTT
AGATCCCCTGGATGCGTTTCCCCAGAAGGGCTTGGAGCCTGGGGACATCGCGGTGCTAGTTCTGTACT
TCCTCTTTGTCCTGGCTGTTGGACTATGGTCCACAGTGAAGACCAAAAGAGACACAGTGAAAGGCTAC
TTCCTGGCTGGAGGGGACATGGTGTGGTGGCCAGTGGGTGCATCCTTGTTTGCCAGCAATGTTGGAAG
TGGACATTTCATTGGCCTGGCAGGGTCAGGTGCTGCTACGGGCATTTCTGTATCAGCTTATGAACTTA
ATGGCTTGTTTTCTGTGCTGATGTTGGCCTGGATCTTCCTACCCATCTACATTGCTGGTCAGGTGACC
ACGATGCCAGAATACCTACGGAAGCGCTTCGGTGGCATCAGAATCCCCATCATCCTGGCTGTACTCTA
CCTATTTATCTACATCTTCACCAAGATCTCGGTAGACATGTATGCAGGTGCCATCTTCATCCAGCAGT
CTTTGCACCTGGATCTGTACCTGGCCATAGTTGGGCTACTGGCCATCACTGCTGTATACACGGTTGCT
GGTGGCCTGGCTGCTGTGATCTACACGGATGCCCTGCAGACGCTGATCATGCTTATAGGAGCGCTCAC
CTTGATGGGCTACAGTTTCGCCGCGGTTGGTGGGATGGAAGGACTGAAGGAGAAGTACTTCTTGGCCC
TGGCTAGCAACCGGAGTGAGAACAGCAGCTGCGGGCTGCCCCGGGAAGATGCCTTCCATATTTTCCGA
GATCCGCTGACATCTGATCTCCCGTGGCCGGGGGTCCTATTTGGAATGTCCATCCCATCCCTCTGGTA
CTGGTGCACGGATCAGGTAATTGTCCAGCGGACTCTGGCTGCCAAGAACCTGTCCCATGCCAAAGGAG
GTGCTCTGATGGCTGCATACCTGAAGGTGCTGCCCCTCTTCATAATGGTGTTCCCTGGGATGGTCAGC
CGCATCCTCTTCCCAGATCAAGTGGCCTGTGCAGATCCAGAGATCTGCCAGAAGATCTGCAGCAACCC
CTCAGGCTGTTCGGACATCGCGTATCCCAAACTCGTGCTGGAACTCCTGCCCACAGGTCTCCGTGGGC
TGATGATGGCTGTGATGGTGGCGGCTCTCATGTCCTCCCTCACCTCCATCTTTAACAGTGCCAGCACC
ATCTTCACCATGGACCTCTGGAATCACCTCCGGCCTCGGGCATCTGAGAAGGAGCTCATGATTGTGGG
CAGGGTGTTTGTGCTGCTGCTGGTCCTGGTCTCCATCCTCTGGATCCCTGTGGTCCAGGCCAGCCAGG
GCGGCCAGCTCTTCATCTATATCCAGTCCATCAGCTCCTACCTGCAGCCGCCTGTGGCGGTGGTCTTC
ATCATGGGATGTTTCTGGAAGAGGACCAATGAAAAGGGTGCCTTCTGGGGCCTGATCTCGGGCCTGCT
CCTGGGCTTGGTTAGGCTGGTCCTGGACTTTATTTACGTGCAGCCTCGATGCGACCAGCCAGATGAGC
GCCCGGTCCTGGTGAAGAGCATTCACTACCTCTACTTCTCCATGATCCTGTCCACGGTCACCCTCATC
ACTGTCTCCACCGTGAGCTGGTTCACAGAGCCACCCTCCAAGGAGATGGTCAGCCACCTGACCTGGTT
TACTCGTCACGACCCCGTGGTCCAGAAGGAACAAGCACCACCAGCAGCTCCCTTGTCTCTTACCCTCT
CTCAGAACGGGATGCCAGAGGCCAGCAGCAGCAGCAGCGTCCAGTTCGAGATGGTTCAAGAAAACACG
TCTAAAACCCACAGCGGTGACATGACCCCAAAGCAGTCCAAAGTGGTGAAGGCCATCCTGTGGCTCTG
TGGAATACAGGAGAAGGGCAAGGAAGAGCTCCCGGCCAGAGCAGAAGCCATCATAGTTTCCCTGGAAG
AAAACCCCTTGGTGAAGACCCTCCTGGACGTCAACCTCATTTTCTGCGTGAGCTGCGCCATCTTTATC
TGGGGCTATTTTGCTTAGTGTGGGGTGAACCCAGGGGTCCAAACTCTGTTTCTCTTCAGTGCTCC

NOV58a, CG56398-01

| | | | |
|---|---|---|---|
| Protein Sequence | SEQ ID NO: 912 | 675 aa | MW at 73989.3kD |

MESGTSSPQPPQLDPLDAFPQKGLEPGDIAVLVLYFLFVLAVGLWSTVKTKRDTVKGYFLAGGDMVWW
PVGASLFASNVGSGHFIGLAGSGAATGISVSAYELNGLFSVLMLAWIFLPIYIAGQVTTMPEYLRKRF
GGIRIPIILAVLYLFIYIFTKISVDMYAGAIFIQQSLHLDLYLAIVGLLAITAVYTVAGGLAAVIYRD
ALQTLIMLIGALTLMGYSFAAVGGMEGLKEKYFLALASNRSENSSCGLPREDAFHIFRDPLTSDLPWP
GVLFGMSIPSLWYWCTDQVIVQRTIAAKNLSHAKGGALMAAYLKVLPLFIMVFPGMVSRILFPDQVAC
ADPEICQKICSNPSGCSDIAYPKLVLELLPTGLRGLMMAVMVAALMSSLTSIFNSASTIFTMDLWNHL
RPRASEKELMIVGRVFVLLLVLVSILWIPVVQASQGGQLFIYIQSISSYLQPPVAVVFIMGCFWKRTN
EKGAFWGLISGLLLGLVRLVLDFIYVQPRCDQPDERPVLVKSIHYLYFSMILSTVTLITVSTVSWFTE
PPSKEMVSHLTWFTRHDPVVQKEQAPPAAPLSLTLSQNGMPEASSSSSVQFEMVQENTSKTHSGDMTP
KQSKVVKAILWLCGIQEKGKEELPARAEAIIVSLEENPLVKTLLDVNLIFCVSCAIFIWGYFA

| | | |
|---|---|---|
| NOV58b, 265726152 | SEQ ID NO: 913 | 1309 bp |

| | | |
|---|---|---|
| DNA Sequence | ORF Start: at 2 | ORF Stop: end of sequence |

CACCGGATCCTACTTCCTGGCTGGAGGGGACATGGTGTGGTGGCCAGTGGGTGCATCCTTGTTTGCCA
GCAATGTTGGAAGTGGACATTTCATTGGCCTGGCAGGGTCAGGTGCTGCTACGGGCATTTCTGTATCA
GCTTATGAACTTAATGGCTTGTTTTCTGTGCTGATGTTGGCCTGGATCTTCCTACCCATCTACATTGC
TGGTCAGGTCACCACGATGCCAGAATACCTACGGAAGCGCTTCGGTGGCATCAGAATCCCCATCATCC
TGGCTGTACTCTACCTATTTATCTACATCTTCACCAAGATCTCGGTAGACATGTATGCAGGTGCCATC
TTCATCCAGCAGTCTTTGCACCTGGATCTGTACCTGGCCATAGTTGGGCTACTGGCCATCACTGCTGT
ATACACGGTTGCTGGTGGCCTGGCTGCTGTGATCTACACGGATGCCCTGCAGACGCTGATCATGCTTA
TAGGAGCGCTCACCTTGATGGGCTACAGTTTCGCCGCGGTTGGTGGGATGGAAGGACTGAAGGAGAAG
TACTTCTTGGCCCTGGCTAGCAACCGGAGTGAGAACAGCAGCTGCGGGCTGCCCCGGGAAGATGCCTT
CCATATTTTCCGAGATCCGCTGACATCTGATCTCCCGTGGCCGGGGGTCCTATTTGGAATGTCCATCC
CATCCCTCTGGTACTGGTGCACGGATCAGGTGATTGTCCAGCGGACTCTGGCTGCCAAGAACCTGTCC
CATGCCAAAGGAGGTGCTCTGATGGCTGCATACCTGAAGGTGCTGCCCCTCTTCATAATGGTGTTCCC
TGGGATGGTCAGCCGCATCCTCTTCCCAGATCAAGTGGCCTGTGCAGATCCAGAGATCTGCCAGAAGA
TCTGCAGCAACCCCTCAGGCTGTTCGGACATCGCGTATCCCAAACTCGTGCTGGAACTCCTGCCCACA
GGGCTCCGTGGGCTGATGATGGCTGTGATGGTGGCGGCTCTCATGTCCTCCCTCACCTCCATCTTTAA
CAGTGCCAGCACCATCTTCACCATGGACCTCTGGAATCACCTCCGGCCTCGGGCATCTGAGAAGGAGC
TCATGATTGTGGGCAGGGTGTTTGTGCTGCTGCTGGTCCTGGTCTCCATCCTCTGGATCCCTGTGGTC
CAGGCCAGCCAGGGCGGCCAGCTCTTCATCTATATCCAGTCCATCAGCTCCTACCTGCAGCCGCCTGT
GGCGGTGGTCTTCATCATGGGATGTTTCTGGAAGAGGACCAATGAAAAGGGTGCCTTCTGGGGCCTGA
TCTCGGGCCTCGAGGGC

NOV58b, 265726152

| | | | |
|---|---|---|---|
| Protein Sequence | SEQ ID NO: 914 | 436 aa | MW at 47358.5kD |

TGSYFLAGGDMVWWPVGASLFASNVGSGHFIGLAGSGAATGISVSAYELNGLFSVLMLAWIFLPIYIA
GQVTTMPEYLRKRFGGIRIPIILAVLYLFIYIFTKISVDMYAGAIFIQQSLHLDLYLAIVGLLAITAV
YTVAGGLAAVIYTDALQTLIMLIGALTLMGYSFAAVGGMEGLKEKYFLALASNRSENSSCGLPREDAF
HIFRDPLTSDLPWPGVLFGMSIPSLWYWCTDQVIVQRTLAAKNLSHAKGGALMAAYLKVLPLFIMVFP
GMVSRILFPDQVACADPEICQKICSNPSGCSDIAYPKLVLELLPTGLRGLMMAVMVAALMSSLTSIFN
SASTIFTMDLWNHLRPRASEKELMIVGRVFVLLLVLVSILWIPVVQASQGGQLFIYIQSISSYLQPPV
AVVFIMGCFWKRTNEKGAFWGLISGLEG

| | | |
|---|---|---|
| NOV58c, 267254040 | SEQ ID NO: 915 | 1912 bp |

| | | |
|---|---|---|
| DNA Sequence | ORF Start: at 2 | ORF Stop: end of sequence |

AGAATTCGCCTTCACCGGATCCAAAAGAGACACAGTGAAAGGCTACTTCCTGGCTGGAGGGGACATGG
TGTGGTGGCCAGTGGGTGCATCCTTGTTTGCCAGCAATGTTGGAAGTGGACATTTCATTGGCCTGGCA
GGGTCAGGTGCTGCTACGGGCATTTCTGTATCAGCTTATGAACTTAATGGCTTGTTTTCTGTGCTGAT
GTTGGCCTGGATCTTCCTACCCATCTACATTGCTGGTCAGGTCACCACGATGCCAGAATACCTACGGA
AGCGCTTCGGTGGCATCAGAATCCCCATCATCCTGGCTGTACTCTACCTATTTATCTACATCTTCACC
AAGATCTCGGTAGACATGTATGCAGGTGCCATCTTCATCCAGCAGTCTTTGCACCTGGATCTGTACCT
GGCCATAGTTGGGCTACTGGCCATCACTGCTGTATACACGGTTGCTGGTGGCCTGGCTGGTGTGATCT
ACACGGATGCCCTGCAGACGCTGATCATGCTTATAGGAGCGCTCACCTTGATGGGCTACAGTTTCGCC
GCGGTTGGTGGGATGGAAGGACTGAAGGAGAAGTACTTCTTGGCCCTGGCTAGCAACCGGAGTGAGAA
CAGCAGCTGCGGGCTGCCCCGGGAAGATGCCTTCCATATTTTCCGAGATCCGCTGACATCTGATCTCC
CGTGGCCGGGGGTCCTATTTGGAATGTCCATCCCATCCCTCTGGTACTGGTGCACGGATCAGGTGATT
GTCCAGCGGACTCTGGCTGCCAAGAACCTGTCCCATGCCAAAGGAGGTGCTCTGATGGCTGCATACCT
GAAGGTGCTGCCCCTCTTCATAATGGTGTTCCCTGGGATGGTCAGCCGCATCCTCTTCCCAGATCAAG
TGGCCTGTGCAGATCCAGAGATCTGCCAGAAGATCTGCAGCAACCCCTCAGGCTGTTCGGACATCGCG
TATCCCAAACTCGTGCTGGAACTCCTGCCCACAGGGCTCCGTGGGCTGATGATGGCTGTGATGGTGGC
GGCTCTCATGTCCTCCCTCACCTCCATCTTTAACAGTGCCAGCACCATCTTCACCATGGACCTCTGGA
ATCACCTCCGGCCTCGGGCATCTGAGAAGGAGCTCATGATTGTGGGCAGGGTGTTTGTGCTGCTGCTG
GTCCTGGTCTCCATCCTCTGGATCCCTGTGGTCCAGGCCAGCCAGGGCGGCCAGCTCTTCATCTATAT
CCAGTCCATCAGCTCCTACCTGCAGCCGCCTGTGGCGGTGGTCTTCATCATGGGATGTTTCTGGAAGA
GGACCAATGAAAAGGGTGCCTTCTGGGGCCTGATCTCGGGCCTGCTCCTGGGCTTGGTTAGGCTGGTC
CTGGACTTTATTTACGTGCAGCCTCGATGCGACCAGCCAGATGAGCGCCCGGTCCTGGTGAAGAGCAT
TCACTACCTCTACTTCTCCATGATCCTGTCCACGGTCACCCTCATCACTGTCTCCACCGTGAGCTGGT
TCACAGAGCCACCCTCCAAGGAGATGGTCAGCCACCTGACCTGGTTTACTCGTCACGACCCCGTGGTC
CAGAAGGAACAAGCACCACCAGCAGCTCCCTTGTCTCTTACCCTCTCTCAGAACGGGATGCCAGAGGC
CAGCAGCAGCAGCAGCGTCCAGTTCGAGATGGTTCAAGAAAACACGTCTAAAACCCACAGCTGTGACA
TGACCCCAAAGCAGTCCAAAGTGGTGAAGGCCATCCTGTGGCTCTGTGGAATACAGGAGAAGGGCAAG
GAAGAGCTCCCGGCCAGAGCAGAAGCCATCATAGTTTCCCTGGAAGAAAACCCCTTGGTGAAGACCCT
CCTGGACGTCAACCTCATTTTCTGCGTGAGCTGCGCCATCTTTATCTGGGGCTATTTTGCTCTCGAGG
GCAGGGCG

NOV58c, 267254040

| | | | |
|---|---|---|---|
| Protein Sequence | SEQ ID NO: 916 | 637 aa | MW at 69945.6kD |

EFAFTGSKRDTVKGYFLAGGDMVWWPVGASLFASNVGSGHFIGLAGSGAATGISVSAYELNGLFSVLM
LAWIFLPIYIAGQVTTMPEYLRKRFGGIRIPIILAVLYLFIYIFTKISVDMYAGAIFIQQSLHLDLYL
AIVGLLAITAVYTVAGGLAAVIYTDALQTLIMLIGALTLMGYSFAAVGGMEGLKEKYFLALASNRSEN
SSCGLPREDAFHIFRDPLTSDLPWPGVLFGMSIPSLWYWCTDQVIVQRTLAAKNLSHAKGGALMAAYL
KVLPLFIMVFPGMVSRILFPDQVACADPEICQKICSNPSGCSDIAYPKLVLELLPTGLRGLMMAVMVA
ALMSSLTSIFNSASTIFTMDLWNHLRPRASEKELMIVGRVFVLLLVLVSIILWIPWQASQGGQLFIYI
QSISSYLQPPVAVVFIMGCFWKRTNEKGAFWGLISGLLLGLVRLVLDFIYVQPRCDQPDERPVLVKSI
HYLYFSMILSTVTLITVSTVSWFTEPPSKEMVSHLTWFTRHDPVVQKEQAPPAAPLSLTLSQNGMPEA
SSSSSVQFEMVQENTSKTHSCDMTPKQSKVVKAILWLCGIQEKGKEELPARAEAIIVSLEENPLVKTL
LDVNLIFCVSCAIFIWGYFALEGRA

| | |
|---|---|
| SEQ ID NO: 917 | 2105 bp |

| | | |
|---|---|---|
| NOV58d, SNP13379242 of | ORF Start: ATG at 31 | ORF Stop: TAG at 2056 |

| | | |
|---|---|---|
| CG56398-01, DNA Sequence | SNP Pos: 804 | SNP Change: C to G |

CCTCAGGATCCAGAGGTCTCGTTCAGGACCATGGAGAGCGGCACCAGCAGCCCTCAGCCTCCACAGTT
AGATCCCCTGGATGCGTTTCCCCAGAAGGGCTTGGAGCCTGGGGACATCGCGGTGCTAGTTCTGTACT
TCCTCTTTGTCCTGGCTGTTGGACTATGGTCCACAGTGAAGACCAAAAGAGACACAGTGAAAGGCTAC
TTCCTGGCTGGAGGGGACATGGTGTGGTGGCCAGTGGGTGCATCCTTGTTTGCCAGCAATGTTGGAAG
TGGACATTTCATTGGCCTGGCAGGGTCAGGTGCTGCTACGGGCATTTCTGTATCAGCTTATGAACTTA
ATGGCTTGTTTTCTGTGCTGATGTTGGCCTGGATCTTCCTACCCATCTACATTGCTGGTCAGGTGACC
ACGATGCCAGAATACCTACGGAAGCGCTTCGGTGGCATCAGAATCCCCATCATCCTGGCTGTACTCTA
CCTATTTATCTACATCTTCACCAAGATCTCGGTAGACATGTATGCAGGTGCCATCTTCATCCAGCAGT
CTTTGCACCTGGATCTGTACCTGGCCATAGTTGGGCTACTGGCCATCACTGCTGTATACACGGTTGCT
GGTGGCCTGGCTGCTGTGATCTACACGGATGCCCTGCAGACGCTGATCATGCTTATAGGAGCGCTCAC
CTTGATGGGCTACAGTTTCGCCGCGGTTGGTGGGATGGAAGGACTGAAGGAGAAGTACTTCTTGGCCC
TGGCTAGCAACCGGAGTGAGAACAGCAGCTGCGGGCTGCCCCGGGAAGATGCCTTGCATATTTTCCGA
GATCCGCTGACATCTGATCTCCCGTGGCCGGGGGTCCTATTTGGAATGTCCATCCCATCCCTCTGGTA
CTGGTGCACGGATCAGGTAATTGTCCAGCGGACTCTGGCTGCCAAGAACCTGTCCCATGCCAAAGGAG
GTGCTCTGATGGCTGCATACCTGAAGGTGCTGCCCCTCTTCATAATGGTGTTCCCTGGGATGGTCAGC
CGCATCCTCTTCCCAGATCAAGTGGCCTGTGCAGATCCAGAGATCTGCCAGAAGATCTGCAGCAACCC
CTCAGGCTGTTCGGACATCGCGTATCCCAAACTCGTGCTGGAACTCCTGCCCACAGGTCTCCGTGGGC
TGATGATGGCTGTGATGGTGGCGGCTCTCATGTCCTCCCTCACCTCCATCTTTAACAGTGCCAGCACC
ATCTTCACCATGGACCTCTGGAATCACCTCCGGCCTCGGGCATCTGAGAAGGAGCTCATGATTGTGGG
CAGGGTGTTTGTGCTGCTGCTGGTCCTGGTCTCCATCCTCTGGATCCCTGTGGTCCAGGCCAGCCAGG
GCGGCCAGCTCTTCATCTATATCCAGTCCATCAGCTCCTACCTGCAGCCGCCTGTGGCGGTGGTCTTC
ATCATGGGATGTTTCTGGAAGAGGACCAATGAAAAGGGTGCCTTCTGGGGCCTGATCTCGGGCCTGCT
CCTGGGCTTGGTTAGGCTGGTCCTGGACTTTATTTACGTGCAGCCTCGATGCGACCAGCCAGATGAGC
GCCCGGTCCTGGTGAAGAGCATTCACTACCTCTACTTCTCCATGATCCTGTCCACGGTCACCCTCATC
ACTGTCTCCACCGTGAGCTGGTTCACAGAGCCACCCTCCAAGGAGATGGTCAGCCACCTGACCTGGTT
TACTCGTCACGACCCCGTGGTCCAGAAGGAACAAGCACCACCAGCAGCTCCCTTGTCTCTTACCCTCT
CTCAGAACGGGATGCCAGAGGCCAGCAGCAGCAGCAGCGTCCAGTTCGAGATGGTTCAAGAAAACACG
TCTAAAACCCACAGCGGTGACATGACCCCAAAGCAGTCCAAAGTGGTGAAGGCCATCCTGTGGCTCTG
TGGAATACAGGAGAAGGGCAAGGAAGAGCTCCCGGCCAGAGCAGAAGCCATCATAGTTTCCCTGGAAG
AAAACCCCTTGGTGAAGACCCTCCTGGACGTCAACCTCATTTTCTGCGTGAGCTGCGCCATCTTTATC
TGGGGCTATTTTGCTTAGTGTGGGGTGAACCCAGGGGTCCAAACTCTGTTTCTCTTCAGTGCTCC

| | | |
|---|---|---|
| NOV58d, SNP13379242 of | SEQ ID NO: 918 | MW at 73955.2kD |

| | | | |
|---|---|---|---|
| CG56398-01, Protein Sequence | SNP Pos: 258 | 675 aa | SNP Change: Phe to Leu |

MESGTSSPQPPQLDPLDAFPQKGLEPGDIAVLVLYFLFVLAVGLWSTVKTKRDTVKGYFLAGGDMVWW
PVGASLFASNVGSGHFIGLAGSGAATGISVSAYELNGLFSVLMLAWIFLPIYIAGQVTTMPEYLRKRF
GGIRIPIILAVLYLFIYIFTKISVDMYAGAIFIQQSLHLDLYLAIVGLLAITAVYTVAGGLAAVIYTD
ALQTLIMLIGALTLMGYSFAAVGGMEGLKEKYFLALASNRSENSSCGLPREDALHIFRDPLTSDLPWP
GVLFGMSIPSLWYWCTDQVIVQRTLAAKNLSHAKGGALMAAYLKVLPLFIMVFPGMVSRILFPDQVAC
ADPEICQKICSNPSGCSDIAYPKLVLELLPTGLRGLMMAVMVAALMSSLTSIFNSASTIFTMDLWNHL
RPRASEKELMIVGRVFVLLLVLVSILWIPVVQASQGGQLFIYIQSISSYLQPPVAVVFIMGCFWKRTN
EKGAFWGLISGLLLGLVRLVLDFIYVQPRCDQPDERPVLVKSIHYLYFSMILSTVTLITVSTVSWFTE
PPSKENVSHLTWFTRHDPVVQKEQAPPAAPLSLTLSQNGMPEASSSSSVQFEMVQENTSKTHSGDMTP
KQSKVVKAILWLCGIQEKGKEELPARAEAIIVSLEENPLVKTLLDVNLIFCVSCAIFIWGYFA

A ClustalW comparison of the above protein sequences yields the following sequence alignment shown in Table 58B.

TABLE 58B
Comparison of the NOV58 protein sequences.

| | |
|---|---|
| NOV58a | MESGTSSPQPPQLDPLDAFPQKGLEPGDIAVLVLYFLFVLAVGLWSTVKTKRDTVKGYFL |
| NOV58b | ------------------------------------------------------TGSYFL |
| NOV58c | -------------------------------------------EFAFTGSKRDTVKGYFL |
| NOV58a | AGGDMVWWPVGASLFASNVGSGHFIGLAGSGAATGISVSAYELNGLFSVLNLAWIFLPIY |
| NOV58b | AGGDMVWWPVGASLFASNVGSGHFIGLAGSGAATGISVSAYELNGLFSVLHLAWIFLPIY |
| NOV58c | AGGDMVWWPVGASLFASNVGSGHFIGLAGSGAATGISVSAYELNGLFSVLMLAWIFLPIY |
| NOV58a | IAGQVTTMPEYLRKRFGGIRIPIILAVLYLFIYIFTKISVDMYAGAIFIQQSLHLDLYLA |
| NOV58b | IAGQVTTMPEYLRKRFGGIRIPIILAVLYLFIYIFTKISVDMYAGAIFIQQSLHLDLYLA |
| NOV58c | IAGQVTTMPEYLRKRFGGIRIPIILAVLYLFIYIFTKISVDMYAGAIFIQQSLHLDLYLA |
| NOV58a | IVGLLAITAVYTVAGGLAAVIYTDALQTLIMLIGALTLMGYSFAAVGGMEGLKEKYFLAL |
| NOV58b | IVGLLAITAVYTVAGGLAAVIYTDALQTLIMLIGALTLMGYSFAAVGGMEGLKEKYFLAL |
| NOV58c | IVGLLAITAVYTVAGGLAAVIYTDALQTLIMLIGALTLMGYSFAAVGGMEGLKEKYFLAL |
| NOV58a | ASNRSENSSCGLPREDAFHIFRDPLTSDLPWPGVLFGMSIPSLWYWCTDQVIVQRTLAAK |
| NOV58b | ASNRSENSSCGLPREDAFHIFRDPLTSDLPWPGVLFGMSIPSLWYWCTDQVIVQRTLAAK |
| NOV58c | ASNRSENSSCGLPREDAFHIFRDPLTSDLPWPGVLFGMSIPSLWYWCTDQVIVQRTLAAK |
| NOV58a | NLSHAKGGALMAAYLKVLPLFIMVFPGMVSRILFPDQVACADPEICQKICSNPSGCSDIA |
| NOV58b | NLSHAKGGALMAAYLKVLPLFIMVFPGMVSRILFPDQVACADPEICQKICSNPSGCSDIA |
| NOV58c | NLSHAKGGALMAAYLKVLPLFIMVFPGMVSRILFPDQVACADPEICQKICSNPSGCSDIA |
| NOV58a | YPKLVLELLPTGLRGLMMAVMVAALMSSLTSIFNSASTIFTMDLWNHLRPRASEKELMIV |
| NOV58b | YPKLVLELLPTGLRGLMMAVMVAALMSSLTSIFNSASTIFTMDLWNHLRPRASEKELMIV |
| NOV58c | YPKLVLELLPTGLRGLMMAVMVAALMSSLTSIFNSASTIFTMDLWNHLRPRASEKELMIV |
| NOV58a | GRVFVLLLVLVSILWIPVVQASQGGQLFIYIQSISSYLQPPVAVVFIMGCFWKRTNEKGA |
| NOV58b | GRVFVLLLVLVSILWIPVVQASQGGQLFIYIQSISSYLQPPVAVVFIMGCFWKRTNEKGA |
| NOV58c | GRVFVLLLVLVSILWIPVVQASQGGQLFIYIQSISSYLQPPVAVVFIMGCFWKRTNEKGA |
| NOV58a | FWGLISGLLLGLVRLVLDFIYVQPRCDQPDERPVLVKSIHYLYFSMILSTVTLITVSTVS |
| NOV58b | FWGLISGLEG-------------------------------------------------- |
| NOV58c | FWGLISGLLLGLVRLVLDFIYVQPRCDQPDERPVLVKSIHYLYFSMILSTVTLITVSTVS |
| NOV58a | WFTEPPSKEMVSHLTWFTRHDPVVQKEQAPPAAPLSLTLSQNGMPEASSSSSVQFEMVQE |
| NOV58b | ------------------------------------------------------------ |
| NOV58c | WFTEPPSKEMVSHLTWFTRHDPVVQKEQAPPAAPLSLTLSQNGMPEASSSSSVQFEMVQE |
| NOV58a | NTSKTHSGDMTPKQSKVVKAILWLCGIQEKGKEELPARAEAIIVSLEENPLVKTLLDVNL |
| NOV58b | ------------------------------------------------------------ |
| NOV58c | NTSKTHSCDMTPKQSKVVKAILWLCGIQEKGKEELPARAEAIIVSLEENPLVKTLLDVNL |
| NOV58a | IFCVSCAIFIWGYFA----- |
| NOV58b | -------------------- |
| NOV58c | IFCVSCAIFIWGYFALEGRA |
| NOV58a | (SEQ ID NO: 912) |
| NOV58b | (SEQ ID NO: 914) |
| NOV58c | (SEQ ID NO: 916) |

Further analysis of the NOV58a protein yielded the following properties shown in Table 58C.

TABLE 58C
Protein Sequence Properties NOV58a

| | |
|---|---|
| SignalP analysis: | Cleavage site between residues 51 and 52 |
| PSORT II analysis: | |

| | |
|---|---|
| PSG: | a new signal peptide prediction method |
| | N-region: length 2; pos.chg 0; neg.chg 1 |
| | H-region: length 11; peak value 0.00 |
| | PSG score: −4.40 |
| GvH: | von Heijne's method for signal seq. recognition |
| | GvH score (threshold: −2.1): −0.94 |
| | possible cleavage site: between 41 and 42 |

>>> Seems to have no N-terminal signal peptide
ALOM: Klein et al's method for TM region allocation

| Init position for calculation: 1 | | |
|---|---|---|
| Tentative number of TMS(s) for the threshold 0.5: 11 | | |
| INTEGRAL | Likelihood = −10.61 | Transmembrane 29-45 |
| INTEGRAL | Likelihood = −6.64 | Transmembrane 106-122 |
| INTEGRAL | Likelihood = −7.64 | Transmembrane 139-155 |
| INTEGRAL | Likelihood = −5.79 | Transmembrane 177-193 |
| INTEGRAL | Likelihood = −3.24 | Transmembrane 210-226 |
| INTEGRAL | Likelihood = −4.88 | Transmembrane 309-325 |
| INTEGRAL | Likelihood = −3.88 | Transmembrane 376-392 |
| INTEGRAL | Likelihood = −13.69 | Transmembrane 423-439 |
| INTEGRAL | Likelihood = −5.47 | Transmembrane 484-500 |
| INTEGRAL | Likelihood = −0.53 | Transmembrane 524-540 |
| INTEGRAL | Likelihood = −5.47 | Transmembrane 654-670 |

| | |
|---|---|
| PERIPHERAL | Likelihood = 1.32 (at 454) |

| | ALOM score: −13.69 (number of TMSs: 11) |
|---|---|
| MTOP: | Prediction of membrane topology (Hartmann et al.) |
| | Center position for calculation: 36 |
| | Charge difference: 6.0 C(3.0) − N(−3.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 4.59 |
| Hyd Moment(95): | 4.89 | G content: | 1 |
| D/E content: | 2 | S/T content: | 4 |
| Score: −6.67 | | | |

Gavel: prediction of cleavage sites for mitochondrial preseq

cleavage site motif not found

NUCDISC: discrimination of nuclear localization signals

pat4: none
pat7: PEYLRKR (3) at 129
bipartite: none
content of basic residues: 6.7%
NLS Score: −0.22

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

66.7%: endoplasmic reticulum
11.1%: vacuolar
11.1%: mitochondrial
11.1%: Golgi

>> prediction for CG56398-01 is end (k = 9)

A search of the NOV58a protein against the Geneseq database, a proprietary database that contains sequences published in patents and patent publication, yielded several homologous proteins shown in Table 58D.

TABLE 58D
Geneseq Results for NOV58a

| | | NOV58a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB80599 | Human sbg1007026SGLT protein - | 1 . . . 675 | 674/675 (99%) | 0.0 |
| | Homo sapiens, 675 aa. | 1 . . . 675 | 674/675 (99%) | |
| | [WO200222802-A1, 21-MAR- | | | |
| | 2002] | | | |
| AAB60093 | Human transport protein TPPT-13 - | 1 . . . 675 | 674/675 (99%) | 0.0 |
| | Homo sapiens, 675 aa. | 1 . . . 675 | 674/675 (99%) | |
| | [WO200078953-A2, 28-DEC- | | | |
| | 2000] | | | |
| AAB85102 | Novel human transporter protein | 1 . . . 675 | 673/675 (99%) | 0.0 |
| | (NHP) - Homo sapiens, 675 aa. | 1 . . . 675 | 673/675 (99%) | |
| | [WO200142469-A1, 14-JUN- | | | |
| | 2001] | | | |
| ABP69833 | Human polypeptide SEQ ID NO | 1 . . . 675 | 672/675 (99%) | 0.0 |
| | 1880 - Homo sapiens, 675 aa. | 1 . . . 675 | 672/675 (99%) | |
| | [WO200270539-A2, 12-SEP- | | | |
| | 2002] | | | |
| AAU77134 | Human sodium-sugar symporter | 1 . . . 675 | 671/675 (99%) | 0.0 |
| | 32620 polypeptide - Homo | 1 . . . 675 | 672/675 (99%) | |
| | sapiens, 675 aa. [WO200216582- | | | |
| | A2, 28-FEB-2002] | | | |

In a BLAST search of public sequence databases, the NOV58a protein was found to have homology to the proteins shown in the BLASTP data in Table 58E.

TABLE 58E
Public BLASTP Results for NOV58a

| | | NOV58a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q8WWX8 | Sodium/glucose cotransporter | 1 . . . 675 | 674/675 (99%) | 0.0 |
| | KST1 - Homo sapiens (Human), | 1 . . . 675 | 674/675 (99%) | |
| | 675 aa. | | | |
| CAC83728 | Sodium-dependent glucose | 1 . . . 675 | 673/675 (99%) | 0.0 |
| | transporter - Homo sapiens | 1 . . . 675 | 673/675 (99%) | |
| | (Human), 675 aa. | | | |
| Q96PP5 | Putative sodium-coupled | 1 . . . 675 | 672/675 (99%) | 0.0 |
| | cotransporter RKST1 - Homo | 1 . . . 675 | 672/675 (99%) | |
| | sapiens (Human), 675 aa. | | | |
| CAD43585 | Sequence 1 from Patent | 1 . . . 675 | 671/675 (99%) | 0.0 |
| | WO0216582 - Homo sapiens | 1 . . . 675 | 672/675 (99%) | |
| | (Human), 675 aa. | | | |
| Q8K0E3 | Similar to putative sodium- | 1 . . . 675 | 583/675 (86%) | 0.0 |
| | coupled cotransporter RKST1 - | 1 . . . 673 | 627/675 (92%) | |
| | Mus musculus (Mouse), 673 aa. | | | |

PFam analysis indicates that the NOV58a protein contains the domains shown in the Table 58F.

TABLE 58F
Domain Analysis of NOV58a

| | NOV58a | Identities/ | |
|---|---|---|---|
| | Match | Similarities | |
| Pfam Domain | Region | for the Matched Region | Expect Value |
| SSF | 58 . . . 487 | 199/447 (45%) | 1.9e−199 |
| | | 370/447 (83%) | |

### Example 59

The NOV59 clone was analyzed, and the nucleotide and encoded polypeptide sequences are shown in Table 59A.

TABLE 59A
NOV59 Sequence Analysis

| | | |
|---|---|---|
| NOV59a, CG56605-03 | SEQ ID NO: 919 | 557 bp |

| | | |
|---|---|---|
| DNA Sequence | ORF Start: ATG at 26 | ORF Stop: TAG at 524 |

ATGGTGGCAGAGTCTACATAGAACTATGCTTCGTGGTGTTCTGGGGAAAACCTTTCGACTTGTTGGCT
ATACTATTCAATATGGCTGTATAGCTCATTGTGCTTTTGAATACGTTGGTGGTGTTGTCATGTGTTCT
GGACCATCAATGGAGCCTACAATTCAAAATTCAGATATTGTCTTTGCAGAAAATCTTAGTCGACATTT
TTATGGTATCCAAAGAGGTGACATTGTGATTGCAAAAAGCCCAAGTGATCCAAAATCAAATATTTGTA
AAAGAGTAATTGGTTTGGAAGGAGACAAAATCCTCACCACTAGTCCATCAGATTTCTTTAAAAGCCAT
AGTTATGTGCCAATGGGTCATGTTTGGTTAGAAGGTGACAATCTACAGAATTCTACAGATTCCAGGTG
CTATGGACCTATTCCATATGGACTAATAAGAGGACGAATCTTCTTTAAGATTTGGCCTCTGAGTGATT
TTGGATTTTTACGTGCCAGCCCTAATGGCCACAGATTTTCTGATGATTAGTAAGCATTTATTCTTTTG
ACTTGATTATTGT

NOV59a, CG56605-03

| | | | |
|---|---|---|---|
| Protein Sequence | SEQ ID NO: 920 | 166 aa | MW at 18504.0kD |

MLRGVLGKTFRLVGYTIQYGCIAHCAFEYVGGVVMCSGPSMETPIQNSDIVFAENLSRHFYGIQRGDI
VIAKSPSDPKSNICKRVIGLEGDKILTTSPSDFFKSHSYVPMGHVWLEGDNLQNSTDSRCYGPIPYGL
IRGRIFFKIWPLSDFGFLRASPNGHRFSDD

| | | |
|---|---|---|
| NOV59b, CG56605-01 | SEQ ID NO: 921 | 513 bp |

| | | |
|---|---|---|
| DNA Sequence | ORF Start: ATG at 16 | ORF Stop: TAA at 445 |

AGTCTACATAGAACTATGCTTCGTGGTGCTCTGGGGAAACCTTCTGACCTTGTTGGCTATGCTATTCA
GTATGGCTGTATAGCTCACTGTGCTTCTGAATACGTTGGTGGTGTTGTCATGTGTTCTGGACCATCAA
TGGAGCCTACAATTCAAAATTCAGATACTGTCTTTGCACAAAATCTTAGTCGACATTTTGATAGTATC
CAAAGAGGTGACATTGTGATTGCAAAAAGCCCAAGTGATCCAACATCAAATATTTGTAAAAGAGTAAC
TGGTTTGGAAGGAGACAAAATCCTCACCACTAGTCCATCAGATTTCTTTAAAAGCTACAGTTATGTCC
CAGTGGGTCATGTTTGGTTAGAAGGTGATAATCTACAGAATTCTACAGATTCCAGTTACTATGGACCT
ATTCCATATGAACTAATAAGAGGACGAATCTTCTTTTAAGATTCGGCCTCTGAGTGATTTTGGATTTT
TATGTGCCAGCCTTAATGGCCACAGATTTTCTGATGA

NOV59b, CG56605-01

| | | | |
|---|---|---|---|
| Protein Sequence | SEQ ID NO: 922 | 143 aa | MW at 15622.4kD |

MLRGAIGKPSDLVGYAIQYGCIAHCASEYVGGVVMCSGPSMEPTIQNSDTVFAQNLSRHFDSIQRGDI
VIAKSPSDPTSNICKRVTGLEGDKILTTSPSDFFKSYSYVPVGHVWLEGDNLQNSTDSSYYGPIPYEL
IRGRIFF

| | | |
|---|---|---|
| NOV59c, CG56605-02 | SEQ ID NO: 923 | 507 bp |

| | | |
|---|---|---|
| DNA Sequence | ORF Start: ATG at 11 | ORF Stop: at 506 |

ACATAGAACTATGCTTCGTGGTGCTCTGGGGAAAACCTTTCGACTTGTTGGCTATACTATTCAATATG
GCTGTATAGCTCATTGTGCTTTTGAATACGTTGGTGGTGTTGTCATGTGTTCTGGACCATCAATGGAG
CCTACAATTCAAAATTCAGATATTGTCTTTGCAGAAAATCTTAGTCGACATTTTTATGGTATCCAAAG
AGGTGACATTGTGATTGCAAAAAGCCCAAGTGATCCAAAATCAAATATTTGTAAAAGAGTAATTGGTT
TGGAAGGAGACAAAATCCTCACCACTAGTCCATCAGATTTCTTTAAAAGCCATAGTTATGTGCCAATG
GGTCATGTTTGGTTAGAAGGTGACAATCTACAGAATTCTACAGATTCCAGGTGCTATGGACCTATTCC
ATATGGACTAATAAGAGGACGAATCTTCTTTAAGATTTGGCCTCTGAGTGATTTTGGATTTTTACGTG
CCAGCCTTAATGGCCACAGATTTTCTGATGA

NOV59c, CG56605-02

| | | | |
|---|---|---|---|
| Protein Sequence | SEQ ID NO: 924 | 165 aa | MW at 18376.9kD |

MLRGALGKTFRLVGYTIQYGCIAHCAFEYVGGVVMCSGPSMEPTIQNSDIVFAENLSRHFYGIQRGDI
VIAKSPSDPKSNICKRVIGLEGDKILTTSPSDFFKSHSYVPMGHVWLEGDNLQNSTDSRCYGPIPYGL
IRGRIFFKIWPLSDFGFLRASLNGHRFSD

A ClustalW comparison of the above protein sequences yields the following sequence alignment shown in Table 59B.

TABLE 59B
Comparison of the NOV59 protein sequences.

| | |
|---|---|
| NOV59a | MLRGVLGKTFRLVGYTIQYGCIAHCAFEYVGGVVMCSGPSMEPTIQNSDIVFAENLSRHF |
| NOV59b | MLRGALGKPSDLVGYAIQYGCIAHCASEYVGGVVMCSGPSMEPTIQNSDTVFAQNLSRHF |
| NOV59c | MLRGVLGKTFRLVGYTIQYGCIAHCAFEYVGGVVMCSGPSMEPTIQNSDIVFAENLSRHF |
| NOV59a | YGIQRGDIVIAKSPSDPKSNICKRVIGLEGDKILTTSPSDFFKSHSYVPMGHVWLEGDNL |
| NOV59b | DSIQRGDIVIAKSPSDPTSNICKRVTGLEGDKILTTSPSDFFKSYSYVPVGHVWLEGDNL |
| NOV59c | YGIQRGDIVIAKSPSDPKSNICKRVIGLEGDKILTTSPSDFFKSHSYVPMGHVWLEGDNL |
| NOV59a | QNSTDSRCYGPIPYGLIRGRIFFKIWPLSDFGFLRASPNGHRFSDD |
| NOV59b | QNSTDSSYYGPIPYELIRGRIFF----------------------- |
| NOV59c | QNSTDSRCYGPIPYGLIRGRIFFKIWPLSDFGFLRASLNGHRFSD- |
| NOV59a | (SEQ ID NO: 920) |
| NOV59b | (SEQ ID NO: 922) |
| NOV59c | (SEQ ID NO: 924) |

Further analysis of the NOV59a protein yielded the following properties shown in Table 59C.

TABLE 59C
Protein Sequence Properties NOV59a

| | |
|---|---|
| SignalP analysis: | No Known Signal Sequence Indicated |

```
PSORT II analysis:
PSG: a new signal peptide prediction method
    N-region: length 11; pos.chg 3; neg.chg 0
    H-region: length 16; peak value 6.29
    PSG score: 1.89
GvH: von Heijne's method for signal seq. recognition
    GvH score (threshold: −2.1): −7.69
    possible cleavage site: between 32 and 33
>>> Seems to have no N-terminal signal peptide
ALOM: Klein et al's method for TM region allocation
    Init position for calculation: 1
    Tentative number of TMS(s) for the threshold 0.5: 0
    number of TMS(s) .. fixed
    PERIPHERAL Likelihood = 1.70 (at 20)
    ALOM score: 1.70 (number of TMSs: 0)
MTOP: Prediction of membrane topology (Hartmann et al.)
    Center position for calculation: 6
    Charge difference: 0.0 C(2.0) − N(2.0)
    N >= C: N-terminal side will be inside
MITDISC: discrimination of mitochondrial targeting seq
```

| | | | |
|---|---|---|---|
| R content: | 2 | Hyd Moment(75): | 14.36 |
| Hyd Moment(95): | 9.31 | G content: | 4 |
| D/E content: | 1 | S/T content: | 2 |
| Score: −2.96 | | | |

```
Gavel: prediction of cleavage sites for mitochondrial preseq
    R-2 motif at 21 FRL|VG
NUCDISC: discrimination of nuclear localization signals
    pat4: none
    pat7: none
    bipartite: none
    content of basic residues: 10.2%
    NLS Score: −0.47
KDEL: ER retention motif in the C-terminus: none
ER Membrane Retention Signals:
    XXRR-like motif in the N-terminus: LRGV
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 89
COIL: Lupas's algorithm to detect coiled-coil regions
    total: 0 residues
Final Results (k = 9/23):
    43.5%: cytoplasmic
    34.8%: mitochondrial
      8.7%: nuclear
      8.7%: peroxisomal
      4.3%: plasma membrane
>> prediction for CG56605-03 is cyt (k = 23)
```

A search of the NOV59a protein against the Geneseq database, a proprietary database that contains sequences published in patents and patent publication, yielded several homologous proteins shown in Table 59D.

TABLE 59D
Geneseq Results for NOV59a

| | | NOV59a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG70899 | Human S26 signal peptidase | 1 . . . 166 | 166/166 (100%) | 2e−97 |
| | family member 33449 - Homo | 1 . . . 166 | 166/166 (100%) | |
| | sapiens, 166 aa. [US2002115630- | | | |
| | A1, 22-AUG-2002] | | | |
| AAB47563 | Protease PRTS-5 - Homo sapiens, | 1 . . . 166 | 166/166 (100%) | 2e−97 |
| | 166 aa. [WO200171004-A2, 27- | 1 . . . 166 | 166/166 (100%) | |
| | SEP-2001] | | | |
| ABG08149 | Novel human diagnostic protein | 1 . . . 166 | 166/166 (100%) | 2e−97 |
| | #8140 - Homo sapiens, 166 aa. | 1 . . . 166 | 166/166 (100%) | |
| | [WO200175067-A2, 11-OCT- | | | |
| | 2001] | | | |
| ABB64326 | Drosophila melanogaster | 5 . . . 150 | 76/160 (47%) | 2e−38 |
| | polypeptide SEQ ID NO 19770 - | 3 . . . 162 | 100/160 (62%) | |
| | Drosophila melanogaster, 166 aa. | | | |
| | [WO200171042-A2, 27-SEP- | | | |
| | 2001] | | | |
| AAB74688 | Human protease and protease | 108 . . . 166 | 58/59 (98%) | 6e−30 |
| | inhibitor PPIM-21 - Homo | 36 . . . 94 | 58/59 (98%) | |
| | sapiens, 94 aa. [WO200110903- | | | |
| | A2, 15-FEB-2001] | | | |

In a BLAST search of public sequence databases, the NOV59a protein was found to have homology to the proteins shown in the BLASTP data in Table 59E.

TABLE 59E
Public BLASTP Results for NOV59a

| | | NOV59a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96LU5 | Hypothetical protein FLJ25059 - | 1 . . . 166 | 166/166 (100%) | 5e−97 |
| | Homo sapiens (Human), 166 aa. | 1 . . . 166 | 166/166 (100%) | |
| Q9CQU8 | 1500034J20Rik protein | 1 . . . 166 | 156/166 (93%) | 1e−89 |
| | (2610528O17Rik protein) | 1 . . . 166 | 157/166 (93%) | |
| | (RIKEN cDNA 1500034J20 gene) | | | |
| | (Unknown EST) - Mus musculus | | | |
| | (Mouse), 166 aa. | | | |
| Q96SH9 | DJ1137O17.1 (Similar to putative | 1 . . . 144 | 144/144 (100%) | 2e−82 |
| | mitochondrial inner membrane | 1 . . . 144 | 144/144 (100%) | |
| | protease subnunit 2) - Homo | | | |
| | sapiens (Human), 144 aa | | | |
| | (fragment). | | | |
| Q9VXR8 | CG9240 protein - Drosophila | 5 . . . 150 | 76/160 (47%) | 5e−38 |
| | melanogaster (Fruit fly), 166 aa. | 3 . . . 162 | 100/160 (62%) | |
| Q8SZ24 | RE22928p - Drosophila | 5 . . . 150 | 76/160 (47%) | 1e−37 |
| | melanogaster (Fruit fly), 166 aa. | 3 . . . 162 | 99/160 (61%) | |

PFam analysis indicates that the NOV59a protein contains the domains shown in the Table 59F.

TABLE 59F
Domain Analysis of NOV59a

| | NOV59a | Identities/ | |
|---|---|---|---|
| | Match | Similarities | |
| Pfam Domain | Region | for the Matched Region | Expect Value |
| Peptidase_S26 | 1 . . . 152 | 37/221 (17%) | 9.1e−07 |
| | | 104/221 (47%) | |

### Example 60

The NOV60 clone was analyzed, and the nucleotide and encoded polypeptide sequences are shown in Table 60A.

TABLE 60A
NOV60 Sequence Analysis

| | | |
|---|---|---|
| NOV60a, CG56645-03 | SEQ ID NO: 925 | 1862 bp |

| | | |
|---|---|---|
| DNA Sequence | ORF Start: ATG at 35 | ORF Stop: TGA at 1829 |

CGGGCTCATACCTAGTGCCTGCGGCAGGACAGCCATGGCCGCCAACTCCACCAGCGACCTCCACACTC
CCGGGACGCAGCTGAGCGTGGCTGACATCATCGTCATCACTGTGTATTTTGCTCTGAACGTGGCCGTG
GGCATATGGTCCTCTTGTCGGGCCAGTAGGAACACGGTGAATGGCTACTTCCTGGCAGGCCGGGACAT
GACGTGGTGGCCGATTGGAGCCTCCCTCTTCGCCAGCAGCGAGGGCTCTGGCCTCTTCATTGGACTGG
CGGGCTCAGGCGCGGCAGGAGGTCTGGCCGTGGCAGGCTTCGAGTGGAATGCCACGTACGTGCTGCTG
GCACTGGCATGGGTGTTCGTGCCCATCTACATCTCCTCAGAGATCGTCACCTTACCTGAGTACATTCA
GAAGCGCTACGGGGGCCAGCGGATCCGCATGTACCTGTCTGTCCTGTCCCTGCTACTGTCTGTCTTCA
CCAAGATATCGCTGGACCTGTACGCGGGGGCTCTGTTTGTGCACATCTGCCTGGGCTGGAACTTCTAC
CTCTCCACCATCCTCACGCTCGGCATCACAGCCCTGTACACCATCGCAGGGGGCCTGGCTGCTGTAAT
CTACACGGACGCCCTGCAGACGCTCATCATGGTGGTGGGGGCTGTCATCCTGACAATCAAAGCTTTTG
ACCAGATCGGTGGTTACGGGCAGCTGGAGGCAGCCTACGCCCAGGCCATTCCCTCCAGGACCATTGCC
AACACCACCTGCCACCTGCCACGTACAGACGCCATGCACATGTTTCGAGACCCCCACACAGGGGACCT
GCCGTGGACCGGGATGACCTTTGGCCTGACCATCATGGCCACCTGGTACTGGTGCACCGACCAGGTCA
TCGTGCAGCGATCACTGTCAGCCCGGGACCTGAACCATGCCAAGGCGGGCTCCATCCTGGCCAGCTAC
CTCAAGATGCTCCCCATGGGCCTGATCATCATGCCGGGCATGATCAGCCGCGCATTGTTCCCAGGTGC
TCATGTCTATGAGGAGAGACACCAAGTGTCCGTCTCTCGAACAGATGATGTGGGCTGCGTGGTGCCGT
CCGAGTGCCTGCGGGCCTGCGGGGCCGAGGTCGGCTGCTCCAACATCGCCTACCCCAAGCTGGTCATG
GAACTGATGCCCATCGGTCTGCGGGGGCTGATGATCGCAGTGATGCTGGCGGCGCTCATGTCGTCGCT
GACCTCCATCTTCAACAGCAGCAGCACCCTCTTCACTATGGACATCTGGAGGCGGCTGCGTCCCCGCT
CCGGCGAGCGGGAGCTCCTGCTGGTGGGACGGCTGGTCATAGTGGCACTCATCGGCGTGAGTGTGGCC
TGGATCCCCGTCCTGCAGGACTCCAACAGCGGGCAACTCTTCATCTACATGCAGTCAGTGACCAGCTC
CCTGGCCCCACCAGTGACTGCAGTCTTTGTCCTGGGCCTGATAGCAGGGCTGGTGGTGGGGGCCACGA
GGCTGGTCCTGGAATTCCTGAACCCAGCCCCACCGTGCGGAGAGCCAGACACGCGGCCAGCCGTCCTG
GGGAGCATCCACTACCTGCACTTCGCTGTCGCCCTCTTTGCACTCAGTGGTGCTGTTGTGGTGGCTGG
AAGCCTGCTGACCCCACCCCCACAGAGTGTCCAGATTGAGAACCTTACCTGGTGGAACCTGGCTCAGG
ATGTGCCCTTGGGAACTAAAGCAGGTGATGGCCAAACACCCCAGAAACACGCCTTCTGGGCCCGTGTC
TGTGGCTTCAATGCCATCCTCCTCATGTGTGTCAACATATTCTTTTATGCCTACTTCGCCTGACACTG
CCATCCTGGACAGAAAGGCAGGAGCT

NOV60a, CG56645-03

| | | | |
|---|---|---|---|
| Protein Sequence | SEQ ID NO: 926 | 598 aa | MW at 64485.7kD |

MAANSTSDLHTPGTQLSVADIIVITVYFALNVAVGIWSSCRASRNTVNGYFLAGRDMTWWPIGASLFA
SSEGSGLFIGLAGSGAAGGLAVAGFEWNATYVLLALAWVFVPIYISSEIVTLPEYIQKRYGGQRIRNY
LSVLSLLLSVFTKISLDLYAGALFVHICLGWNFYLSTILTLGITALYTIAGGLAAVIYTDALQTLIMV
VGAVILTIKAFDQIGGYGQLEAAYAQAIPSRTIANTTCHLPRTDAMHMFRDPHTGDLPWTGMTFGLTI
MATWYWCTDQVIVQRSLSARDLNHAKAGSILASYLKMLPMGLIIMPGMISRALFPGAHVYEERHQVSV
SRTDDVGCVVPSECLRACGAEVGCSNIAYPKLVMELMPIGLRGLMIAVMLAALMSSLTSIFNSSSTLF
TMDIWRRLRPRSGERELLLVGRLVIVALIGVSVAWIPVLQDSNSGQLFIYMQSVTSSLAPPVTAVFVL
GLIAGLVVGATRLVLEFLNPAPPCGEPDTRPAVLGSIHYLHFAVALFALSGAVVVAGSLLTPPPQSVQ
IENLTWWNLAQDVPLGTKAGDGQTPQKHAFWARVCGFNAILLMCVNIFFYAYFA

| | | |
|---|---|---|
| NOV60b, CG56645-04 | SEQ ID NO: 927 | 1801 bp |

| | | |
|---|---|---|
| DNA Sequence | ORF Start: ATG at 35 | ORF Stop: TGA at 1790 |

CGGGCTCATACCTAGTGCCTGCGGCAGGACAGCCATGGCCGCCAACTCCACCAGCGACCTCCACACTC
CCGGGACGCAGCTGAGCGTGGCTGACATCATCGTCATCACTGTGTATTTTGCTCTGAACGTGGCCGTG
GGCATATGGTCCTCTTGTCGGGCCAGTAGGAACACGGTGAATGGCTACTTCCTGGCAGGCCGGGACAT
GACGTGGTGGCCGGATTGGAGCCTCCCTCTTCGCCAGCGCGAGGGCTCTGGCCTCTTCATTGGACTGG
CGGGCTCAGGCGCGGCAGGAGGTCTGGCCGTGGCAGGCTTCGAGTGGAATGCCACGTACGTGCTGCTG
GCACTGGCATGGGTGTTCGTGCCCATCTACATCTCCTCAGAGATCGTCACCTTACCTGAGTACATTCA
GAAGCGCTACGGGGGCCAGCGGATCCGCATGTACCTGTCTGTCCTGTCCCTGCTACTGTCTGTCTTCA
CCAAGATATCGCTGGACCTGTACGCGGGGGCTCTGTTTGTGCACATCTGCCTGGGCTGGAACTTCTAC
CTCTCCACCATCCTCACGCTCGGCATCACAGCCCTGTACACCATCGCAGCTTTTGACCAGATCGGTGG
TTACGGGCAGCTGGAGGCAGCCTACGCCCAGGCCATTCCCTCCAGGACCATTGCCAACACCACCTGCC
AGCTGCCACGTACAGACGCCATGCACATGTTTCGAGACCCCCACACAGGGGACCTGCCGTGGACCGGG
ATGACCTTTGGCCTGACCATCATGGCCACCTGGTACTGGTGCACCGACCAGGTCATCGTGGAGGGATC
ACTGTCAGCCCGGGACCTGAACCATGCCAAGGGGGGCTCCATCCTGGGCAGCTACCTCAAGATGCTCC
CCATGGGCCTGATCATCATGCCGGGCATGATCAGCCGCGCATTGTTCCCAGGTGCTCATGTCTATGAG
GAGAGACACCAAGTGTCCGTCTCTCGAACAGATGATGTGGGCTGCGTGGTGCCGTCCGAGTGCCTGCG
GGCCTGCGGGGCCGAGGTCGGCTGCTCCAACATCGCCTACCCCAAGCTGGTCATGGAACTGATGCCCA
TCGGTCTGCGGGGGCTGATGATCGCAGTGATGCTGGCGGCGCTCATGTCGTCGCTGACCTCCATCTTC
AACAGCAGCAGCACCCTCTTCACTATGGACATCTGGAGGCGGCTGCGTCCCCGCTCCGGCGAGCGGGA
GCTCCTGCTGGTGGGACGGCTGGTCATAGTGGCACTCATCGGCGTGAGTGTGGCCTGGATCCCCGTCC
TGCAGGACTCCAACAGCGGGCAACTCTTCATCTACATGCAGTCAGTGACCAGCTCCCTGGCCCCACCA
GTGACTGCAGTCTTTGTCCTGGGCGTCTTCTGGCGACGTGCCAACGAGCAGGGGGCCTTCTGGGGCCT
GATAGCAGGGCTGGTGGTGGGGGCCACGAGGCTGGTCCTGGAATTCCTGAACCCGGCCCCACCGTGCG
GAGAGCCAGACACGCGGCCAGCCGTCCTGGGGAGCATCCACTACCTGCACTTCGCTGTCGCCCTCTTT
GCACTCAGTGGTGCTGTTGTGGTGGCTGGAAGCCTGCTGACCCCACCCCCACAGAGTGTCCAGATTGA
GAACCTTACCTGGTGGACCCTGGCTCAGGATGTGCCCTTGGGAACTAAAGCAGGTGATGGCCAAACAC
CCCAGAAACACGCCTTCTGGGCCCGTGTCTGTGGCTTCAATGCCATCCTCCTCATGTGTGTCAACATA
TTCTTTTATGCCTACTTCGCCTGACACTGCCAT

NOV60b, CG56645-04

| | | | |
|---|---|---|---|
| Protein Sequence | SEQ ID NO: 928 | 585 aa | MW at 63317.1kD |

MAANSTSDLHTPGTQLSVADIIVITVYFALNVAVGIWSSCRASRNTVNGYFLAGRDMTWWPIGASLFA
SSEGSGLFIGLAGSGAAGGLAVAGFEWNATYVLLALANVFVPIYISSEIVTLPEYIQKRYGGQRIRMY
LSVLSLLLSVFTKISLDLYAGALFVHICLGWNFYLSTILTLGITALYTIAAFDQIGGYGOLEAAYAQA
IPSRTIANTTCQLPRTDAMHMFRDPHTGDLPWTGMTFGLTIMATWYWCTDQVIVEGSLSARDLNHAKG
GSILGSYLKMLPMGLIIMPGMISRALFPGAHVYEERHQVSVSRTDDVGCVVPSECLRACGAEVGCSNI
AYPKLVMELMPIGLRGLMIAVMLAALMSSLTSIFNSSSTLFTMDIWRRLRPRSGERELLLVGRLVIVA
LIGVSVAWIPVLQDSNSGQLFIYMQSVTSSLAPPVTAVFVLGVFWRRANEQGAFWGLIAGLVVGATRL
VLEFLNPAPPCGEPDTRPAVLGSIHYLHFAVALFALSGAVVVAGSLLTPPPQSVQIENLTWWTLAQDV
PLGTKAGDGQTPQKHAFWARVCGFNAILLMCVNIFFYAYFA

| | | |
|---|---|---|
| NOV60c, CG56645-01 | SEQ ID NO: 929 | 1914 bp |

| | | |
|---|---|---|
| DNA Sequence | ORF Start: ATG at 51 | ORF Stop: TGA at 1839 |

TTGCCCCTCAGTCCCTCGGGCTCATACCTAGTGCCTGCGGCAGGACAGCCATGGCCGCCAACTCCACC
AGCGACCTCCACACTCCCGGGACGCAGCTGAGCGTGGCTGACATCATCGTCATCACTGTGTATTTTGC
TCTGAACGTGGCCGTGGGCATATGGTCCTCTTGTCGGGCCAGTAGGAACACGGTGAATGGCTACTTCC
TGGCAGGCCGGGACATGACGTGGTGGCCGATTGGAGCCTCCCTCTTCGCCAGCAGCGAGGGCTCTGGC
CTCTTCATTGGACTGGCGGGCTCAGGCGCGGCAGGAGGTCTGGCCGTGGCAGGCTTCGAGTGGAATGC
CACGTACGTGCTGCTGGCACTGGCATGGGTGTTCGTGCCCATCTACATCTCCTCAGAGATCGTCACCT
TACCTGAGTACATTCAGAAGCGCTACGGGGGCCAGCGGATCCGCATGTACCTGTCTGTCCTGTCCCTG
CTACTGTCTGTCTTCACCAAGATATCGCTGGACCTGTACGCGGGGGCTCTGTTTGTGCACATCTGCCT
GGGCTGGAACTTCTACCTCTCCACCATCCTCACGCTCGGCATCACAGCCCTGTACACCATCGCAGGGG
GCCTGGCTGCTGTAATCTACACGGACGCCCTGCAGACGCTCATCATGGTGGTGGGGGCTGTCATCCTG
ACAATCAAAGCTTTTGACCAGATCGGTGGTTACGGGCAGCTGGAGGCAGCCTACGCCCAGGCCATTCC
CTCCAGGACCATTGCCAACACCACCTGCCACCTGCCACGTACAGACGCCATGCACATGTTTCGAGACC
CCCACACAGGGGACCTGCCGTGGACCGGGATGACCTTTGGCCTGACCATCATGGCCACCTGGTACTGG
TGCACCGACCAGGTGATCGTGCAGCGATCACTGTCAGCCCGGGACCTGAACCATGCCAAGGCGGGCTC
CATCCTGGCCAGCTACCTCAAGATGCTCCCCATGGGCCTGATCATAATGCCGGGCATGATCAGCCGCG
CATTGTTCCCAGATGATGTGGGCTGCGTGGTGCCGTCCGAGTGCCTGCGGGCCTGCGGGGCCGAGGTC
GGCTGCTCCAACATCGCCTACCCCAAGCTGGTCATGGAACTGATGCCCATCGGTCTGCGGGGGCTGAT
GATCGCAGTGATGCTGGCGGCGCTCATGTCGTCGCTGACCTCCATCTTCAACAGCAGCAGCACCCTCT
TCACTATGGACATCTGGAGGCGGCTGCGTCCCCGCTCCGGCGAGCGGGAGCTCCTGCTGGTGGGACGG
CTGGTCATAGTGGCACTCATCGGCGTGAGTGTGGCCTGGATCCCCGTCCTGCAGGACTCCAACAGCGG
GCAACTCTTCATCTACATGCAGTCAGTGACCAGCTCCCTGGCCCCACCAGTGACTGCAGTCTTTGTCC
TGGGCGTCTTCTGGCGACGTGCCAACGAGCAGGGGGCCTTCTGGGGCCTGATAGCAGGGCTGGTGGTG
GGGGCCACGAGGCTGGTCCTGGAATTCCTGAACCCAGCCCCACCGTGCGGAGAGCCAGACACGCGGCC
AGCCGTCCTGGGGAGCATCCACTACCTGCACTTCGCTGTCGCCCTCTTTGCACTCAGTGGTGCTGTTG
TGGTGGCTGGAAGCCTGCTGACCCCACCCCCACAGAGTGTCCAGATTGAGAACCTTACCTGGTGGACC
CTGGCTCAGGATGTGCCCTTGGGAACTAAAGCAGGTGATGGCCAAACACCCCAGAAACACGCCTTCTG
GGCCCGTGTCTGTGGCTTCAATGCCATCCTCCTCATGTGTGTCAACATATTCTTTTATGCCTACTTCG
CCTGACACTGCCATCCTGGACAGAAAGGCAGGAGCTCTGAGTCCTCAGGTCCACCCATTTCCCTCATG
GGGATCCCGA

NOV60c, CG56645-01

| | | | |
|---|---|---|---|
| Protein Sequence | SEQ ID NO: 930 | 596 aa | MW at 64341.6kD |

MAANSTSDLHTPGTQLSVADIIVITVYFALNVAVGIWSSCRASRNTVNGYFLAGRDMTWWPIGASLFA
SSEGSGLFIGLAGSGAAGGLAVAGFEWNATYVLLALAWVFVPIYISSEIVTLPEYIQKRYGGQRIRNY
LSVLSLLLSVFTKISLDLYAGALFVHICLGWNFYLSTILTLGITAIYTIAGGLAAVIYTDALQTLIMV
VGAVILTIKAFDQIGGYGQLEAAYAQAIPSRTIANTTCHLPRTDAMHMFRDPHTGDLPWTGMTFGLTI
MATWYWCTDQVIVQRSLSARDLNHAKAGSILASYLKMLPMGLIIMPGMISRALFPDDVGCVVPSECLR
ACGAEVGCSNIAYPKLVMELMPIGLRGLMIAVMLAALMSSLTSIFNSSSTLFTMDIWRRLRPRSGERE
LLLVGRLVIVALIGVSVAWIPVLQDSNSGQLFIYMQSVTSSLAPPVTAVFVLGVFWRRANEQGAFWGL
IAGLVVGATRLVLEFLNPAPPCGEPDTRPAVLGSIHYLHFAVALFALSGAVVVAGSLLTPPPQSVQIE
NLTWWTLAQDVPLGTKAGDGQTPQKIIAFWARVCGFNAILLMCVNIFFYAYFA

| | | |
|---|---|---|
| NOV60d, CG56645-02 | SEQ ID NO: 931 | 1912 bp |

| | | |
|---|---|---|
| DNA Sequence | ORF Start: ATG at 35 | ORF Stop: TGA at 1871 |

CGGGCTCATACCTAGTGCCTGCGGCAGGACAGCCATGGCCGCCAACTCCACCAGCGACCTCCACACTC
CCGGGACGCAGCTGAGCGTGGCTGACATCATCGTCATCACTGTGTATTTTGCTCTGAACGTGGCCGTG
GGCATATGGTCCTCTTGTCGGGCCAGTAGGAACACGGTGAATGGCTACTTCCTGGCAGGCCGGGACAT
GACGTGGTGGCCGATTGGAGCCTCCCTCTTCGCCAGCAGCGAGGGCTCTGGCCTCTTCATTGGACTGC
CGGGCTCAGGCGCGGCAGGAGGTCTGGCCGTGGCAGGCTTCGAGTGGAATGCCACGTACGTGCTGCTG
GCACTGGCATGGGTGTTCGTGCCCATCTACATCTCCTCAGAGATCGTCACCTTACCTGAGTACATTCA
GAAGCGCTACGGGGGCCAGCGGATCCGCATGTACCTGTCTGTCCTGTCCCTGCTACTGTCTGTCTTCA
CCAAGATATCGCTGGACCTGTACGCGGGGGCTCTGTTTGTGCACATCTGCCTGGGCTGGAACTTCTAC
CTCTCCACCATCCTCACGCTCGGCATCACAGCCCTGTACACCATCGCAGGGGGCCTGGCTGCTGTAAT
CTACACGGACGCCCTGCAGACGCTCATCATGGTGGTGGGGGCTGTCATCCTGACAATCAAAGCTTTTG
ACCAGATCGGTGGTTACGGGCAGCTGGAGGCAGCCTACGCCCAGGCCATTCCCTCCAGGACCATTGCC
AACACCACCTGCCACCTGCCACGTACAGACGCCATGCACATGTTTCGAGACCCCCACACAGGGGACCT
GCCGTGGACCGGGATGACCTTTGGCCTGACCATCATGGCCACCTGGTACTGGTGCACCGACCAGGTCA
TCGTGCAGCGATCACTGTCAGCCCGGGACCTGAACCATGCCAAGGCGGGCTCCATCCTGGCCAGCTAC
CTCAAGATGCTCCCCATGGGCCTGATCATCATGCCGGGCATGATCAGCCGCGCATTGTTCCCAGGTGC
TCATGTCTATGAGGAGAGACACCAAGTGTCCGTCTCTCGAACAGATGATGTGGGCTGCGTGGTGCCGT
CCGAGTGCCTGCGGGCCTGCGGGGCCGAGGTCGGCTGCTCCAACATCGCCTACCCCAAGCTGGTCATG
GAACTGATGCCCATCGGTCTGCGGGGGCTGATGATCGCAGTGATGCTGGCGGCGCTCATGTCGTCGCT
GACCTCCATCTTCAACAGCAGCAGCACCCTCTTCACTATGGACATCTGGAGGCGGCTGCGTCCCCGCT
CCGGCGAGCGGGAGCTCCTGCTGGTGGGACGGCTGGTCATAGTGGCACTCATCGGCGTGAGTGTGGCC
TGGATCCCCGTCCTGCAGGACTCCAACAGCGGGCAACTCTTCATCTACATGCAGTCAGTGACCAGCTC
CCTGGCCCCACCAGTGACTGCAGTCTTTGTCCTGGGCGTCTTCTGGCGACGTGCCAACGAGCAGGGGG
CCTTCTGGGGCCTGATAGCAGGGCTGGTGGTGGGGGCCACGAGGCTGGTCCTGGAATTCCTGAACCCA
GCCCCACCGTGCGGAGAGCCAGACACGCGGCCAGCCGTCCTGGGGAGCATCCACTACCTGCACTTCGC
TGTCGCCCTCTTTGCACTCAGTGGTGCTGTTGTGGTGGCTGGAAGCCTGCTGACCCCACCCCCACAGA
GTGTCCAGATTGAGAACCTTACCTGGTGGACCCTGGCTCAGGATGTGCCCTTGGGAACTAAAGCAGGT
GATGGCCAAACACTCCAGAAACACGCCTTCTGGGCCCGTGTCTGTGGCTTCAATGCCATCCTCCTCAT
GTGTGTCAACATATTCTTTTATGCCTACTTCGCCTGACACTGCCATCCTGGACAGAAAGGCAGGAGCT
CTGAGTCC

NOV60d, CG56645-02

| | | | |
|---|---|---|---|
| Protein Sequence | SEQ ID NO: 932 | 612 aa | MW at 66194.7kD |

MAANSTSDLHTPGTQLSVADIIVITVYFALNVAVGIWSSCRASRNTVNGYFLAGRDMTWWPIGASLFA
SSEGSGLFIGLAGSGAAGGLAVAGFEWNATYVLLAIAWVFVPIYISSEIVTLPEYIQKRYGGQRIRMY
LSVLSLLLSVFTKISLDLYAGALFVHICLGWNFYLSTILTLGITALYTIAGGLAAVIYTDALQTLIMV
VGAVILTIKAFDQIGGYGQLEAAYAQAIPSRTIANTTCHLPRTDAMHMFRDPHTGDLPWTGMTFGLTI
MATWYWCTDQVIVQRSLSARDLNHAKAGSSILASYLKMLPNGLIIMPGMISRALFPGANVYEERHQVSV
SRTDDVGCVVPSECLRACGAEVGCSNIAYPKLVMELMPIGLRGLMIAVMLAALMSSLTSIFNSSSTLF
TMDIWRRLRPRSGERELLLVGRLVIVALIGVSVAWIPVLQDSNSGQLFIYMQSVTSSLAPPVTAVFVL
GVFWRRANEQGAFWGLIAGLVVGATRLVLEFLNPAPPCGEPDTRPAVLGSIHYLHFAVALFALSGAVV
VAGSLLTPPPQSVQIENLTWWTLAQDVPLGTKAGDGQTLQKHAFWARVCGFNAILLMCVMIFFYAYFA

A ClustalW comparison of the above protein sequences yields the following sequence alignment shown in Table 60B.

TABLE 60B
Comparison of the NOV60 protein sequences.

| | |
|---|---|
| NOV60a | MAANSTSDLHTPGTQLSVADIIVITVYFALNVAVGIWSSCRASRNTVNGYFLAGRDMTWW |
| NOV60b | MAANSTSDLHTPGTQLSVADIIVITVYFALNVAVGIWSSCRASRNTVNGYFLAGRDMTWW |
| NOV60c | MAANSTSDLHTPGTQLSVADIIVITVYFALNVAVGIWSSCRASRNTVNGYFLAGRDMTWW |
| NOV60d | MAANSTSDLHTPGTQLSVADIIVITVYFALNVAVGIWSSCRASRNTVNGYFLAGRDMTWW |
| NOV60a | PIGASLFASSEGSGLFIGLAGSGAAGGLAVAGFEWNATYVLLALAWVFVPIYISSEIVTL |
| NOV60b | PIGASLFASSEGSGLFIGLAGSGAAGGLAVAGFEWNATYVLLALAWVFVPIYISSEIVTL |
| NOV60c | PIGASLFASSEGSGLFIGLAGSGAAGGLAVAGFEWNATYVLLALAWVFVPIYISSEIVTL |
| NOV60d | PIGASLFASSEGSGLFIGLAGSGAAGGLAVAGFEWNATYVLLALAWVFVPIYISSEIVTL |
| NOV60a | PEYIQKRYGGQRIRMYLSVLSLLLSVFTKISLDLYAGALFVHICLGWNFYLSTILTLGIT |
| NOV60b | PEYIQKRYGGQRIRMYLSVLSLLLSVFTKISLDLYAGALFVHICLGWNFYLSTILTLGIT |
| NOV60c | PEYIQKRYGGQRIRMYLSVLSLLLSVFTKISLDLYAGALFVHICLGWNFYLSTILTLGIT |
| NOV60d | PEYIQKRYGGQRIRMYLSVLSLLLSVFTKISLDLYAGALFVHICLGWNFYLSTILTLGIT |
| NOV60a | ALYTIAGGLAAVIYTDALQTLIMVVGAVILTIKAFDQIGGYGQLEAAYAQAIPSRTIANT |
| NOV60b | ALYTIA---------------------------AFDQIGGYGQLEAAYAQAIPSRTIANT |
| NOV60c | ALYTIAGGLAAVIYTDALQTLIMVVGAVILTIKAFDQIGGYGQLEAAYAQAIPSRTIANT |
| NOV60d | ALYTIAGGLAAVIYTDALQTLIMVVGAVILTIKAFDQIGGYGQLEAAYAQAIPSRTIANT |
| NOV60a | TCHLPRTDAMHMFRDPHTGDLPWTGMTFGLTIMATWYWCTDQVIVQRSLSARDLNHAKAG |
| NOV60b | TCQLPRTDAMHMFRDPHTGDLPWTGMTFGLTIMATWYWCTDQVIVEGSLSARDLNHAKAG |
| NOV60c | TCHLPRTDAMHMFRDPHTGDLPWTGMTFGLTIMATWYWCTDQVIVQRSLSARDLNHAKAG |
| NOV60d | TCHLPRTDAMHMFRDPHTGDLPWTGMTFGLTIMATWYWCTDQVIVQRSLSARDLNHAKAG |
| NOV60a | SILASYLKMLPMGLIIMPGMISRALFPGAHVYEERHQVSVSRTDDVGCVVPSECLRACGA |
| NOV60b | SILASYLKMLPMGLIIMPGMISRALFPGAHVYEERHQVSVSRTDDVGCVVPSECLRACGA |
| NOV60c | SILASYLKMLPMGLIIMPGMISRALFP----------------DDVGCVVPSECLRACGA |
| NOV60d | SILASYLKMLPMGLIIMPGMISRALFPGAHVYEERHQVSVSRTDDVGCVVPSECLRACGA |
| NOV60a | EVGCSNIAYPKLVMELMPIGLRGLMIAVMLAALMSSLTSIFNSSSTLFTMDIWRRLRPRS |
| NOV60b | EVGCSNIAYPKLVMELMPIGLRGLMIAVMLAALMSSLTSIFNSSSTLFTMDIWRRLRPRS |
| NOV60c | EVGCSNIAYPKLVMELMPIGLRGLMIAVMLAALMSSLTSIFNSSSTLFTMDIWRRLRPRS |
| NOV60d | EVGCSNIAYPKLVMELMPIGLRGLMIAVMLAALMSSLTSIFNSSSTLFTMDIWRRLRPRS |
| NOV60a | GERELLLVGRLVIVALIGVSVAWIPVLQDSNSGQLFIYMQSVTSSLAPPVTAVFVL---- |
| NOV60b | GERELLLVGRLVIVALIGVSVAWIPVLQDSNSGQLFIYMQSVTSSLAPPVTAVFVLGVFW |
| NOV60c | GERELLLVGRLVIVALIGVSVAWIPVLQDSNSGQLFIYMQSVTSSLAPPVTAVFVLGVFW |
| NOV60d | GERELLLVGRLVIVALIGVSVAWIPVLQDSNSGQLFIYMQSVTSSLAPPVTAVFVLGVFW |
| NOV60a | ----------GLIAGLVVGATRLVLEFLNPAPPCGEPDTRPAVLGSIHYLHFAVALFALS |
| NOV60b | RRANEQGAFWGLIAGLVVGATRLVLEFLNPAPPCGEPDTRPAVLGSIHYLHFAVALFALS |
| NOV60c | RRANEQGAFWGLIAGLVVGATRLVLEFLNPAPPCGEPDTRPAVLGSIHYLHFAVALFALS |
| NOV60d | RRANEQGAFWGLIAGLVVGATRLVLEFLNPAPPCGEPDTRPAVLGSIHYLHFAVALFALS |
| NOV60a | GAVVVAGSLLTPPPQSVQIENLTWWTLAQDVPLGTKAGDGQTPQKHAFWARVCGFNAILL |
| NOV60b | GAVVVAGSLLTPPPQSVQIENLTWWTLAQDVPLGTKAGDGQTPQKHAFWARVCGFNAILL |
| NOV60c | GAVVVAGSLLTPPPQSVQIENLTWWTLAQDVPLGTKAGDGQTPQKHAFWARVCGFNAILL |
| NOV60d | GAVVVAGSLLTPPPQSVQIENLTWWTLAQDVPLGTKAGDGQTPQKHAFWARVCGFNAILL |
| NOV60a | MCVNIFFYAYFA |
| NOV60b | MCVNIFFYAYFA |
| NOV60c | MCVNIFFYAYFA |
| NOV60d | MCVNIFFYAYFA |
| NOV60a | (SEQ ID NO: 926) |
| NOV60b | (SEQ ID NO: 928) |
| NOV60c | (SEQ ID NO: 930) |
| NOV60d | (SEQ ID NO: 932) |

Further analysis of the NOV60a protein yielded the following properties shown in Table 60C.

TABLE 60C
Protein Sequence Properties NOV60a

| | |
|---|---|
| SignalP analysis: | Cleavage site between residues 43 and 44 |

```
PSORT II analysis:
PSG: a new signal peptide prediction method
    N-region: length 8; pos.chg 0; neg.chg 1
    H-region: length 11; peak value 0.00
    PSG score: −4.40
GvH: von Heijne's method for signal seq. recognition
    GvH score (threshold: −2.1): −2.19
    possible cleavage site: between 42 and 43
>>> Seems to have no N-terminal signal peptide
    ALOM: Klein et al's method for TM region allocation
    Init position for calculation: 1
    Tentative number of TMS(s) for the threshold 0.5: 12
```

| | | |
|---|---|---|
| INTEGRAL | Likelihood = −6.00 | Transmembrane 21-37 |
| INTEGRAL | Likelihood = −1.17 | Transmembrane 75-91 |
| INTEGRAL | Likelihood = −4.51 | Transmembrane 97-113 |
| INTEGRAL | Likelihood = −1.33 | Transmembrane 136-152 |
| INTEGRAL | Likelihood = −2.92 | Transmembrane 177-193 |
| INTEGRAL | Likelihood = −3.88 | Transmembrane 196-212 |
| INTEGRAL | Likelihood = −0.27 | Transmembrane 301-317 |
| INTEGRAL | Likelihood = −5.04 | Transmembrane 384-400 |
| INTEGRAL | Likelihood = −10.03 | Transmembrane 431-447 |
| INTEGRAL | Likelihood = −8.65 | Transmembrane 470-486 |
| INTEGRAL | Likelihood = −6.74 | Transmembrane 520-536 |
| INTEGRAL | Likelihood = −3.45 | Transmembrane 578-594 |

| | |
|---|---|
| PERIPHERAL | Likelihood = 0.63 (at 159) |

```
    ALOM score: −10.03 (number of TMSs: 12)
MTOP: Prediction of membrane topology (Hartmann et al.)
    Center position for calculation: 28
    Charge difference: 4.5 C(3.0) − N(−1.5)
    C > N: C-terminal side will be inside
>>> membrane topology: type 3b
MITDISC: discrimination of mitochondrial targeting seq
```

| | | | |
|---|---|---|---|
| R content: | 0 | Hyd Moment(75): | 8.68 |
| Hyd Moment(95): | 8.07 | G content: | 1 |
| D/E content: | 2 | S/T content: | 6 |
| Score: −5.13 | | | |

```
Gavel: prediction of cleavage sites for mitochondrial preseq
    cleavage site motif not found
NUCDISC: discrimination of nuclear localization signals
    pat4: none
    pat7: none
    bipartite: none
    content of basic residues: 5.5%
    NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 94.1
COIL: Lupas's algorithm to detect coiled-coil regions
    total: 0 residues
Final Results (k = 9/23):
    77.8%: endoplasmic reticulum
    11.1%: mitochondrial
    11.1%: vacuolar
>> prediction for CG56645-03 is end (k = 9)
```

A search of the NOV60a protein against the Geneseq database, a proprietary database that contains sequences published in patents and patent publication, yielded several homologous proteins shown in Table 60D.

TABLE 60D
Geneseq Results for NOV60a

| | | NOV60a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB99496 | Amino acid sequence of | 1 . . . 598 | 597/612 (97%) | 0.0 |
| | sodium/glucose cotransporter | 48 . . . 659 | 597/612 (97%) | |
| | Fbh68723pat - Homo sapiens, 664 | | | |
| | aa. [WO200283857-A2, 24-OCT- | | | |
| | 2002] | | | |
| ABB99497 | Amino acid sequence of | 1 . . . 598 | 581/612 (94%) | 0.0 |
| | sodium/glucose cotransporter | 48 . . . 643 | 581/612 (94%) | |
| | h68723 - Homo sapiens, 643 aa. | | | |
| | [WO200283857-A2, 24-OCT- | | | |
| | 2002] | | | |
| AAO14199 | Human transporter and ion | 1 . . . 598 | 581/612 (94%) | 0.0 |
| | channel TRICH-16 - Homo | 1 . . . 596 | 581/612 (94%) | |
| | sapiens, 596 aa. [WO200204520- | | | |
| | A2, 17-JAN-2002] | | | |
| ABB80588 | Human sbg1020829SGLT protein - | 1 . . . 598 | 581/612 (94%) | 0.0 |
| | Homo sapiens, 596 aa. | 1 . . . 596 | 581/612 (94%) | |
| | [WO200222802-A1, 21-MAR- | | | |
| | 2002] | | | |
| ABG31594 | Human transporter protein - Homo | 1 . . . 598 | 581/612 (94%) | 0.0 |
| | sapiens, 596 aa. [US2002081678- | 1 . . . 596 | 581/612 (94%) | |
| | A1, 27-JUN-2002] | | | |

In a BLAST search of public sequence databases, the NOV60a protein was found to have homology to the proteins shown in the BLASTP data in Table 60E.

TABLE 60E
Public BLASTP Results for NOV60a

| | | NOV60a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC51188 | Sequence 144 from Patent | 1 . . . 598 | 581/612 (94%) | 0.0 |
| | WO0149728 - Homo sapiens | 1 . . . 596 | 581/612 (94%) | |
| | (Human), 596 aa. | | | |
| Q28610 | Na+/glucose cotransporter- | 1 . . . 598 | 518/612 (84%) | 0.0 |
| | related protein - Oryctolagus | 2 . . . 597 | 544/612 (88%) | |
| | cuniculus (Rabbit), 597 aa. | | | |
| Q96LQ1 | Hypothetical protein FLJ25217 - | 157 . . . 598 | 441/456 (96%) | 0.0 |
| | Homo sapiens (Human), 517 aa. | 62 . . . 517 | 441/456 (96%) | |
| Q8BZW1 | Weakly similar to NA+-glucose | 6 . . . 551 | 339/560 (60%) | 0.0 |
| | cotransporter type 1 - Mus | 19 . . . 562 | 425/560 (75%) | |
| | musculus (Mouse), 685 aa. | | | |
| Q8BGU9 | Weakly similar to NA+-glucose | 6 . . . 551 | 338/560 (60%) | 0.0 |
| | cotransporter type 1 - Mus | 19 . . . 562 | 424/560 (75%) | |
| | musculus (Mouse), 685 aa. | | | |

PFam analysis indicates that the NOV60a protein contains the domains shown in the Table 60F.

TABLE 60F
Domain Analysis of NOV60a

| | NOV60a | Identities/ | |
|---|---|---|---|
| | Match | Similarities | |
| Pfam Domain | Region | for the Matched Region | Expect Value |
| SSF | 50 . . . 485 | 187/463 (40%) | 1.3e−167 |
| | | 362/463 (78%) | |

### Example 61

The NOV61 clone was analyzed, and the nucleotide and encoded polypeptide sequences are shown in Table 61A.

TABLE 61A
NOV61 Sequence Analysis

| | | |
|---|---|---|
| NOV61a, CG56667-01 | SEQ ID NO: 933 | 1027 bp |

| | | |
|---|---|---|
| DNA Sequence | ORF Start: ATG at 1 | ORF Stop: TAG at 940 |

ATGATCTGCTCAGCTATCAACCTACACTTACTACTGGCAGTTAAGATGATTCACCCTGTCTGGATTCT
TTGCAGTCATCACTCTCGATCAGCATCTTCACACACCCATGTACTTCTTCCTGAAGAACCTCTCCGTT
TTGGATCTGTGCTACATCTCAGTCACTGTGCCTAAATCCATCCGTAACTCCCTGACTCGCAGAAGCTC
CATCTCTTATCTTGGCTGTGTGGCTCAAGTCTATTTTTTCTCTGCCTTTGCATCTGCTGAGCTGGCCT
TCCTTACTGTCATGTCTTATGACCGCTATGTTGCCATTTGCCACCCCCTCCAATACAGAGCCGTGATG
ACATCAGGAGGGTGCTATCAGATGGCAGTCACCACCTGGCTAAGCTGCTTTTCCTACGCAGCCGTCCA
CACTGGCAACATGTTTCGGGAGCACGTTTGCAGATCCAGTGTGATCCACCAGTTCTTCCGTGACATCC
CTCATGTGTTGGCCCTGGTTTCCTGTGAGGTTTTCTTTGTAGAGTTTTTGACCCTGGCCCTGAGCTCA
TGCTTGGTTCTGGGATGCTTTATTCTCATGATGATCTCCTATTTCCAAATCTTCTCAACGGTGCTCAG
AATCCCTTCAGGACAGAGTCGAGCAAAAGCCTTCTCCACCTGCTCCCCCCAGCTCATTGTCATCATGC
TCTTTCTTACCACAGGGCTCTTTGCTGCCTTAGGACCAATTGCAAAAGCTCTGTCCATTCAGGATTTA
GTGATTGCTCTGACATACACAGTTTTGCCTCCCTTCCTCAATCCCATCATATATAGTCTTAGGAATAA
GGAGATTAAAACAGCCATGTGGAGACTCTTTGTGAAGATATATTTTCTGCAAAAGTAGAACATCCTGG
TCTTTACTATAGAAGATCTGCAACAAAACCCCAAAAAAGCATAAATACTTTATGACAAAAAAAGATGA
AAAAATT

NOV61a, CG56667-01

| | | | |
|---|---|---|---|
| Protein Sequence | SEQ ID NO: 934 | 313 aa | MW at 35319.9kD |

MICSAINLHLLLAVKMIHPVWILAPREQGLFLLIYLAVLVGNLLIIAVITLDQHLHTPMYFFLKNLSV
LDLCYISVTVPKSIRNSLTRRSSISYLGCVAQVYFFSAFASAELAFLTVMSYDRYVAICHPLQYRAVM
TSGGCYQMAVTTWLSCFSYAAVHTGNMFREHVCRSSVIHQFFRDIPHVLALVSCEVFFVEFLTLALSS
CLVLGCFILMMISYFQIFSTVLRIPSGQSRAKAFSTCSPQLIVIMLFLTTGLFAALGPIAKALSIQDL
VIALTYTVLPPFLNPIIYSLRNKEIKTANWRLFVKIYFLQK

| | | |
|---|---|---|
| NOV61b, 268952113 | SEQ ID NO: 935 | 817 bp |

| | | |
|---|---|---|
| DNA Sequence | ORF Start: at 2 | ORF Stop: end of sequence |

CACCGGATCCGTCATCACTCTCGATCAGCATCTTCACACACCCATGTACTTCTTCCTGAAGAACCTCT
CTGTTTTGGATCTGTGCTACATCTCAGTCACTGTGCCTAAATCCATCCGTAACTCCCTGACTCGCAGA
AGCTCCATCTCTTATCTTGGCTGTGTGGCTCAAGTCTATTTTTTCTCTGCCTTTGCATCTGCTGAGCT
GGCCTTCCTTACTGTCATGTCTTATGACCGCTATGTTGCCATTTGCCACCCCCTCCAATACAGAGCCG
TGATGACATCAGGAGGGTGCTATCAGATGGCAGTCACCACCTGGCTAAGCTGCTTTTCCTACGCAGCC
GTCCACACTGGCAACATGTTTCGGGAGCACGTTTGCAGATCCAATGTGATCCACCAGTTCTTCCGTGA
CATCCCTCATGTGTTGGCCCTGGTTTCCTGTGAGGTTTTCTTTGTAGAGTTTTTGACCCTGGCCCTGA
GCTCATGCTTGGTTCTGGGATGCTTTATTCTCATGATGATCTCCTATTTCCAAATCTTCTCAACGGTG
CTCAGAATCCCTTCAGGACAGAGTCGAGCAAAAGCCTTCTCCACCTGCTCCCCCCAGCTCATTGTCAT
CATGCTCTTTCTTACCACAGGGCTCTTTGCTGCCTTAGGACCAATTGCAAAAGCTCTGTCCATTCAGG
ATTTAGTGATTGCTCTGACATACACAGTTTTGCCTCCCTTCCTCAATCCCATCATATATAGTCTTAGG
AAATAAGGAGATTAAACAGCCATGTGGAGACTCTTTGTGAAGATATATTTTCTGCAAAAGCTCGAGGG
C

NOV61b, 268952113

| | | | |
|---|---|---|---|
| Protein Sequence | SEQ ID NO: 936 | 272 aa | MW at 30688.0kD |

TGSVITLDQHLHTPNYFFLKNLSVLDLCYISVTVPKSIRNSLTRRSSISYLGCVAQVYFFSAFASAEL
AFLTVMSYDRYVAICHPLQYRAVMTSGGCYQMAVTTWLSCFSYAAVHTGNMFREHVCRSNVIHQFFRD
IPHVLALVSCEVFFVEFLTLALSSCLVLGCFILMMISYFQIFSTVLRIPSGQSRAKAFSTCSPQLIVI
MLFLTTGLFAALGPIAKALSIQDLVIALTYTVLPPFLNPIIYSLRNKEIKTAMWRLFVKIYFLQKLEG

A ClustalW comparison of the above protein sequences yields the following sequence alignment shown in Table 61B.

TABLE 61B
Comparison of the NOV61 protein sequences.

| | |
|---|---|
| NOV61a | MICSAINLHLLLAVKMIHPVWILAPREQGLFLLIYLAVLVGNLLIIAVITLDQHLHTPMY |
| NOV61b | --------------------------------------------TGSVITLDQHLHTPMY |
| NOV61a | FFLKNLSVLDLCYISVTVPKSIRNSLTRRSSISYLGCVAQVYFFSAFASAELAFLTVMSY |
| NOV61b | FFLKNLSVLDLCYISVTVPKSIRNSLTRRSSISYLGCVAQVYFFSAFASAELAFLTVMSY |
| NOV61a | DRYVAICHPLQYRAVMTSGGCYQMAVTTWLSCFSYAAVHTGNMFREHVCRSSVIHQFFRD |
| NOV61b | DRYVAICHPLQYRAVMTSGGCYQMAVTTWLSCFSYAAVHTGNMFREHVCRSNVIHQFFRD |
| NOV61a | IPHVLALVSCEVFFVEFLTLALSSCLVLGCFILMMISYFQIFSTVLRIPSGQSRAKAFST |
| NOV61b | IPHVLALVSCEVFFVEFLTLALSSCLVLGCFILMMISYFQIFSTVLRIPSGQSRAKAFST |
| NOV61a | CSPQLIVIMLFLTTGLFAALGPIAKALSIQDLVIALTYTVLPPFLNPIIYSLRNKEIKTA |
| NOV61b | CSPQLIVIMLFLTTGLFAALGPIAKALSIQDLVIALTYTVLPPFLNPIIYSLRNKEIKTA |
| NOV61a | MWRLFVKIYFLQK--- |
| NOV61b | MWRLFVKIYFLQKLEG |
| NOV61a | (SEQ ID NO: 934) |
| NOV61b | (SEQ ID NO: 936) |

Further analysis of the NOV61a protein yielded the following properties shown in Table 61C.

TABLE 61C
Protein Sequence Properties NOV61a

| | |
|---|---|
| SignalP analysis: | Cleavage site between residues 48 and 49 |
| PSORT II analysis: | |

```
PSG: a new signal peptide prediction method
    N-region: length 0; pos.chg 0; neg.chg 0
    H-region: length 14; peak value 6.18
    PSG score: 1.78
GvH: von Heijne's method for signal seq. recognition
    GvH score (threshold: −2.1): −3.68
    possible cleavage site: between 41 and 42
>>> Seems to have no N-terminal signal peptide
ALOM: Klein et al's method for TM region allocation
    Init position for calculation: 1
    Tentative number of TMS(s) for the threshold 0.5: 7
```

| | | |
|---|---|---|
| INTEGRAL | Likelihood = −2.39 | Transmembrane 1-17 |
| INTEGRAL | Likelihood = −11.20 | Transmembrane 33-49 |
| INTEGRAL | Likelihood = −0.22 | Transmembrane 101-117 |
| INTEGRAL | Likelihood = −1.22 | Transmembrane 183-199 |
| INTEGRAL | Likelihood = −8.86 | Transmembrane 200-216 |
| INTEGRAL | Likelihood = −7.54 | Transmembrane 245-261 |
| INTEGRAL | Likelihood = −0.00 | Transmembrane 273-289 |

| | |
|---|---|
| PERIPHERAL | Likelihood = 2.01 (at 62) |

```
    ALOM score: −11.20 (number of TMSs: 7)
MTOP: Prediction of membrane topology (Hartmann et al.)
    Center position for calculation: 8
    Charge difference: 0.5 C(2.0) − N(1.5)
    C > N: C-terminal side will be inside
>>>Caution: Inconsistent mtop result with signal peptide
>>> membrane topology: type 3b
MITDISC: discrimination of mitochondrial targeting seq
```

| | | | |
|---|---|---|---|
| R content: | 1 | Hyd Moment(75): | 1.74 |
| Hyd Moment(95): | 4.81 | G content: | 0 |
| D/E content: | 1 | S/T content: | 1 |
| Score: −4.54 | | | |

```
Gavel: prediction of cleavage sites for mitochondrial preseq
    R-2 motif at 99 RRS|SI
NUCDISC: discrimination of nuclear localization signals
    pat4: none
    pat7: none
    bipartite: none
    content of basic residues: 7.0%
    NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 94.1
COIL: Lupas's algorithm to detect coiled-coil regions
    total: 0 residues
Final Results (k = 9/23):
    55.6%: endoplasmic reticulum
    11.1%: mitochondrial
    11.1%: vacuolar
    11.1%: vesicles of secretory system
    11.1%: Golgi
>> prediction for CG56667-01 is end (k = 9)
```

A search of the NOV61a protein against the Geneseq database, a proprietary database that contains sequences published in patents and patent publication, yielded several homologous proteins shown in Table 61D.

TABLE 61D
Geneseq Results for NOV61a

| | | NOV61a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG72316 | Human olfactory receptor | 47 . . . 219 | 149/173 (86%) | 2e−80 |
| | polypeptide, SEQ ID NO: 1997 - | 1 . . . 173 | 151/173 (87%) | |
| | Homo sapiens, 177 aa. | | | |
| | [WO200127158-A2, 19-APR- | | | |
| | 2001] | | | |
| ABB77320 | Human G-protein coupled | 30 . . . 304 | 148/275 (53%) | 8e−78 |
| | receptor SEQ ID NO 4 - Homo | 28 . . . 301 | 189/275 (67%) | |
| | sapiens, 309 aa. [WO200198323- | | | |
| | A2, 27-DEC-2001] | | | |
| AAU95571 | Human olfactory and pheromone | 30 . . . 304 | 148/275 (53%) | 8e−78 |
| | G protein-coupled receptor #58 - | 28 . . . 301 | 189/275 (67%) | |
| | Homo sapiens, 309 aa. | | | |
| | [WO200224726-A2, 28-MAR- | | | |
| | 2002] | | | |
| ABP51618 | Human G protein coupled receptor | 30 . . . 304 | 149/275 (54%) | 8e−78 |
| | SEQ ID NO: 118 - Homo sapiens, | 28 . . . 300 | 191/275 (69%) | |
| | 308 aa. [WO200250276-A2, 27- | | | |
| | JUN-2002] | | | |
| ABG76784 | Human G-protein coupled | 30 . . . 304 | 148/275 (53%) | 8e−78 |
| | receptor (GPCR) protein #18 - | 28 . . . 301 | 189/275 (67%) | |
| | Homo sapiens, 309 aa. | | | |
| | [WO200259313-A2, 01-AUG- | | | |
| | 2002] | | | |

In a BLAST search of public sequence databases, the NOV61a protein was found to have homology to the proteins shown in the BLASTP data in Table 61E.

TABLE 61E
Public BLASTP Results for NOV61a

| | | NOV61a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NHC8 | Seven transmembrane helix | 20 . . . 307 | 164/291 (56%) | 3e−81 |
| | receptor - Homo sapiens | 18 . . . 308 | 200/291 (68%) | |
| | (Human), 309 aa. | | | |
| Q8VF67 | Olfactory receptor MOR218-3 - | 30 . . . 305 | 146/276 (52%) | 1e−78 |
| | Mus musculus (Mouse), 315 aa. | 32 . . . 307 | 182/276 (65%) | |
| CAD57961 | Sequence 117 from Patent | 30 . . . 304 | 149/275 (54%) | 2e−77 |
| | WO0250276 - Homo sapiens | 28 . . . 300 | 191/275 (69%) | |
| | (Human), 308 aa. | | | |
| Q8NHC5 | Seven transmembrane helix | 30 . . . 304 | 148/275 (53%) | 2e−77 |
| | receptor - Homo sapiens | 28 . . . 301 | 189/275 (67%) | |
| | (Human), 309 aa. | | | |
| Q8VEU5 | Olfactory receptor MOR218-8 - | 30 . . . 313 | 143/284 (50%) | 4e−75 |
| | Mus musculus (Mouse), 320 aa. | 27 . . . 310 | 185/284 (64%) | |

PFam analysis indicates that the NOV61a protein contains the domains shown in the Table 61F.

TABLE 61F
Domain Analysis of NOV61a

| | NOV61a | Identities/ | |
|---|---|---|---|
| | Match | Similarities | |
| Pfam Domain | Region | for the Matched Region | Expect Value |
| 7tm_1 | 41 . . . 290 | 53/278 (19%) | 3.9e−31 |
| | | 174/278 (63%) | |

### Example 62

The NOV62 clone was analyzed, and the nucleotide and encoded polypeptide sequences are shown in Table 62A.

TABLE 62A
NOV62 Sequence Analysis

| | | |
|---|---|---|
| NOV62a, CG56868-01 | SEQ ID NO: 937 | 2422 bp |

| | | |
|---|---|---|
| DNA Sequence | ORF Start: ATG at 51 | ORF Stop: TAG at 2382 |

GAACTCCTTTTCTCAAGCACTTCTGCTCTCCTCTACCAGAATCACTCAGAATGCTTCCCGGGTGTATA
TTCTTGATGATTTTACTCATTCCTCAGGTTAAAGAAAAGTTCATCCTTGGAGTAGAGGGTCAACAACT
GGTTCGTCCTAAAAAGCTTCCTCTGATACAGAAGCGAGATACTGGACACACCCATGATGATGACATAC
TGAAAACGTATGAAGAAGAATTGTTGTATGAAATAAAACTAAATAGAAAAACCTTAGTCCTTCATCTT
CTAAGATCCAGGGAGTTCCTAGGCTCAAATTACAGTGAAACATTCTACTCCATGAAAGGAGAAGCGTT
CACCAGGCATCCTCAGATCATGGAACACTGTTACTATAAAGGAAACATCCTAAATGAAAAGAATTCTG
TTGCCAGCATCAGTACTTGTGACGGGTTGAGGGGATTCTTCAGAATAAACGACCAAAGATACCTCATT
GAACCAGTGAAATACTCAGATGAGGGAGAACATTTGGTGTTCAAATATAACCTGAGGGTGCCGTATGG
TGCCAATTATTCCTGTACAGAGCTTAATTTTACCAGAAAAACTGTTCCAGGGGATAATGAATCTGAAG
AAGACTCCAAAATAAAAGGCATCCATGATGAAAAGTATGTTGAATTGTTCATTGTTGCTGATGATACT
GTGTATCGCAGAAATGGTCATCCTCACAATAAACTAAGGAACCGAATTTGGGGAATGGTCAATTTTGT
CAACATGATTTATAAAACCTTAAACATCCATGTGACGTTGGTTGGCATTGAAATATGGACACATGAAG
ATAAAATAGAACTATATTCAAATATAGAAACTACCTTATTGCGTTTTTCATTTTGGCAAGAAAAGATC
CTTAAAACACGGAAGGATTTTGATCATGTTGTATTACTCAGTGGGAAGTGGCTCTACTCACATGTGCA
AGGAATTTCTTATCCAGGGGGTATGTGCCTGCCCTATTATTCCACCAGTATCATTAAGGATCTTTTAC
CTGACACAAACATAATTGCAAACAGAATGGCACATCAACTGGGGCATAACCTTGGGATGCAGCATGAC
GAGTTCCCATGCACCTGTCCTTCAGGAAAATGCGTGATGGACAGTGATGGAAGCATTCCTGCACTGAA
ATTCAGTAAATGCAGCCAAAACCAATACCACCAGTACTTGAAGGATTATAAGCCAACATGCATGCTCA
ACATTCCATTTCCTTACAATTTTCATGATTTCCAATTrTGTGGAAACAAGAAGTTGGATGAGGGTGAA
GAGTGTGACTGTGGCCCTGCTCAGGAGTGTACTAATCCTTGCTGTGATGCACACACATGTGTACTGAA
GCCAGGATTTACTTGTGCAGAAGGAGAATGCTGTGAATCTTGTCAGATAAAAAAAGCAGGGTCCATAT
GCAGACCGGCGAAAGATGAATGTGATTTTCCTGAGATGTGCACTGGCCACTCGCCTGCCTGTCCTAAG
GACCAGTTCAGGGTCAATGGATTTCCTTGCAAGAACTCAGAAGGCTACTGTTTCATGGGGAAATGTCC
AACTCGTGAGGATCAGTGCTCTGAACTATTTGATGATGAGGCAATAGAGAGTCATGATATCTGCTACA
AGATGAATACAAAAGGAAATAAATTTGGATACTGCAAAAACAAGGAAAACAGATTTCTTCCCTGTGAG
GAGAAGGATGTCAGATGTGGAAAGATCTACTGCACTGGAGGGGAGCTTTCCTCTCTCCTTGGAGAAGA
CAAGACTTATCACCTTAAGGATCCCCAGAAGAATGCTACTGTCAAATGCAAAACTATTTTTTTATACC
ATGATTCTACAGACATTGGCCTGGTGGCGTCAGGAACAAAATGTGGAGAGGGAATGGTGTGCAACAAT
GGTGAATGTCTAAACATGGAAAAGGTCTATATCTCAACCAATTGCCCCTCTCAGTGCAATGAAAATCC
TGTGGATGGCCACGGACTCCAGTGCCACTGTGAGGAAGGACAGGCACCTGTAGCCTGTGAAGAAACCT
TACATGTTACCAGTATCACCATCTTGGTTGTTGTGCTTGTCCTGGTTATTGTCGGTATCGGAGTTCTT
ATACTATTAGTTCGTTACCGAAAATGTATCAAGTTGAAGCAAGTTCAGAGCCCACCTACAGAAACCCT
GGGAGTGGAGAACAAAGGATACTTTGGTGATGAGCAGCAGATAAGGACTGAGCCAATCCTGCCAGAAA
TTCATTTCCTAAATCAGAGAACTCCAGAATCCTTGGAAAGCCTGCCCACTAGTTTTTCAAGTCCCCAC
TACATCACACTGAAACCTGCAAGTAAAGATTCAAGAGGAATCGCAGATCCCAATCAAAGTGCCAAGTG
GTAGGTTACCCTGACAGATAGTACCTCCCTTTTTTATTTTTC

NOV62a, CG56868-01

| | | | |
|---|---|---|---|
| Protein Sequence | SEQ ID NO: 938 | 777 aa | MW at 88341.2kD |

MLPGCIFLMILLIPQVKEKFILGVEGQQLVRPKKLPLIQKRDTGHTHDDDILKTYEEELLYEIKLNRK
TLVLNLLRSREFLGSNYSETFYSMKGEAFTRHPQIMEHCYYKGNILNEKNSVASISTCDGLRGFFRIN
DQRYLIEPVKYSDEGEHLVFKYNLRVPYGANYSCTELNFTRKTVPGDNESEEDSKIKGIHDEKYVELF
IVADDTVYRRNGHPHNKLRNRIWGMVNFVNNIYKTLNIHVTLVGIEIWTHEDKIELYSNIETTLLRFS
FWQEKILKTRKDFDHVVLLSGKWLYSHVQGISYPGGMCLPYYSTSIIKDLLPDTNIIANRMAHQLGHN
LGNQHDEFPCTCPSGKCVMDSDGSIPALKFSKCSQNQYHQYLKDYKPTCMLNIPFPYNFHDFQFCGNK
KLDEGEECDCGPAQECTNPCCDAHTCVLKPGFTCAEGECCESCQIKKAGSICRPAKDECDFPEMCTGH
SPACPKDQFRVNGFPCKNSEGYCFMGKCPTREDQCSELFDDEAIESHDICYKMNTKGNKFGYCKNKEN
RFLPCEEKDVRCGKIYCTGGELSSLLGEDKTYHLKDPQKNATVKCKTIFLYHDSTDIGLVASGTKCGE
GMVCNNGECLNMEKVYISTNCPSQCNENPVDGHGLQCHCEEGQAPVACEETLHVTSITILVVVLVLVI
VGIGVLILLVRYRKCIKLKQVQSPPTETLGVENKGYFGDEQQIRTEPILPEIHFLNQRTPESLESLPT
SFSSPHYITLKPASKDSRGIADPNQSAKW

| | | |
|---|---|---|
| NOV62b, 276580332 | SEQ ID NO: 939 | 247 bp |

| | | |
|---|---|---|
| DNA Sequence | ORF Start: at 2 | ORF Stop: end of sequence |

CACCGGTACCGATGAGGGTGAAGAGTGTGACTGTGGCCCTGCTCAGGAGTGTACTAATCCTTGCTGTG
ATGCACACACATGTGTACTGAAGCCAGGATTTACTTGTGCAGAAGGAGAATGCTGTGAATCTTGTCAG
ATAAAAAAAGCAGGGTCCATATGCAGACCGGCGAAAGATGAATGTGATTTTCCTGAGATGTGCACTGG
CCACTCGCCTGCCTGTCCTAAGGACCAGTTCAGGGTCGACGGC

NOV62b, 276580332

| | | | |
|---|---|---|---|
| Protein Sequence | SEQ ID NO: 940 | 82 aa | MW at 8696.6kD |

TGTDEGEECDCGPAQECTNPCCDAHTCVLKPGFTCAEGECCESCQIKKAGSICRPAKDECDFPEMCTG
HSPACPKDQFRVDG

A ClustalW comparison of the above protein sequences yields the following sequence alignment shown in Table 62B.

TABLE 62B
Comparison of the NOV62 protein sequences.

| | |
|---|---|
| NOV62a | MLPGCIFLNILLIPQVKEKFILGVEGQQLVRPKKLPLIQKRDTGHTHDDDILKTYEEELL |
| NOV62b | ------------------------------------------------------------ |
| NOV62a | YEIKLNRKTLVLHLLRSREFLGSNYSETFYSMKGEAFTRHPQIMEHCYYKGNILNEKNSV |
| NOV62b | ------------------------------------------------------------ |
| NOV62a | ASISTCDGLRGFFRINDQRYLIEPVKYSDEGEHLVFKYNLRVPYGANYSCTELNFTRKTV |
| NOV62b | ------------------------------------------------------------ |
| NOV62a | PGDNESEEDSKIKGIHDEKYVELFIVADDTVYRRNGHPHNKLRNRIWGMVNFVNMIYKTL |
| NOV62b | ------------------------------------------------------------ |
| NOV62a | NIHVTLVGIEIWTHEDKIELYSNIETTLLRFSFWQEKILKTRKDFDHVVLLSGKWLYSHV |
| NOV62b | ------------------------------------------------------------ |
| NOV62a | QGISYPGGMCLPYYSTSIIKDLLPDTNIIANRMAHQLGHNLGMQHDEFPCTCPSGKCVMD |
| NOV62b | ------------------------------------------------------------ |
| NOV62a | SDGSIPALKFSKCSQNQYHQYLKDYKPTCMLNIPFPYNFHDFQFCGNKKLDEGEECDCGP |
| NOV62b | -----------------------------------------------TGTDEGEECDCGP |
| NOV62a | AQECTNPCCDAHTCVLKPGFTCAEGECCESCQIKKAGSICRPAKDECDFPEMCTGHSPAC |
| NOV62b | AQECTNPCCDAHTCVLKPGFTCAEGECCESCQIKKAGSICRPAKDECDFPEMCTGHSPAC |
| NOV62a | PKDQFRVNGFPCKNSEGYCFMGKCPTREDQCSELFDDEAIESHDICYKMNTKGNKFGYCK |
| NOV62b | PKDQFRVDG--------------------------------------------------- |
| NOV62a | NKENRFLPCEEKDVRCGKIYCTGGELSSLLGEDKTYHLKDPQKNATVKCKTIFLYHDSTD |
| NOV62b | ------------------------------------------------------------ |
| NOV62a | IGLVASGTKCGEGMVCNNGECLNNEKVYISTNCPSQCNENPVDGHGLQCHCEEGQAPVAC |
| NOV62b | ------------------------------------------------------------ |
| NOV62a | EETLHVTSITILVVVLVLVIVGIGVLILLVRYRKCIKLKQVQSPPTETLGVENKGYFGDE |
| NOV62b | ------------------------------------------------------------ |
| NOV62a | QQIRTEPILPEIHFLNQRTPESLESLPTSFSSPHYITLKPASKDSRGIADPNQSAKW |
| NOV62b | --------------------------------------------------------- |
| NOV62a | (SEQ ID NO: 938) |
| NOV62b | (SEQ ID NO: 940) |

Further analysis of the NOV62a protein yielded the following properties shown in Table 62C.

TABLE 62C
Protein Sequence Properties NOV62a

| | |
|---|---|
| SignalP analysis: | Cleavage site between residues 19 and 20 |
| PSORT II analysis: | |

```
PSG: a new signal peptide prediction method
    N-region: length 0; pos.chg 0; neg.chg 0
    H-region: length 16; peak value 12.97
PSG sc ore: 8.57
GvH: von Heijne's method for signal seq. recognition
    GvH score (threshold: −2.1): −5.58
    possible cleavage site: between 18 and 19
>>> Seems to have no N-terminal signal peptide
ALOM: Klein et al's method for TM region allocation
    Init position for calculation: 1
    Tentative number of TMS(s) for the threshold 0.5: 2
```

| | | |
|---|---|---|
| INTEGRAL | Likelihood = −2.18 | Transmembrane 1-17 |
| INTEGRAL | Likelihood = −18.52 | Transmembrane 671-687 |

| | |
|---|---|
| PERIPHERAL | Likelihood = 4.35 (at 235) |

```
    ALOM score: −18.52 (number of TMSs: 2)
MTOP: Prediction of membrane topology (Hartmann et al.)
    Center position for calculation: 8
    Charge difference: 0.0 C(1.0) − N(1.0)
    N >= C: N-terminal side will be inside
>>> membrane topology: type 3a
MITDISC: discrimination of mitochondrial targeting seq
```

| | | | |
|---|---|---|---|
| R content: | 0 | Hyd Moment(75): | 3.80 |
| Hyd Moment(95): | 1.48 | G content: | 1 |
| D/E content: | 1 | S/T content: | 0 |
| Score: −6.33 | | | |

```
Gavel: prediction of cleavage sites for mitochondrial preseq
    cleavage site motif not found
NUCDISC: discrimination of nuclear localization signals
    pat4: RPKK (4) at 31
    pat7: none
    bipartite: none
    content of basic residues: 11.3%
    NLS Score: −0.22
KDEL: ER retention motif in the C-terminus: none
ER Membrane Retention Signals:
    KKXX-like motif in the C-terminus: QSAK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
    Bacterial regulatory proteins, gntR family signature (PS00043):
***found***
        EEELLYEIKLNRKTLVLHLLRS at 56
NNCN: Reinhardt's method for Cytoplasmic/Nuclear discrimination
    Prediction: nuclear
    Reliability: 55.5
COIL: Lupas's algorithm to detect coiled-coil regions
    total: 0 residues
Final Results (k = 9/23):
    34.8%: nuclear
    30.4%: endoplasmic reticulum
    21.7%: mitochondrial
      4.3%: vesicles of secretory system
      4.3%: cytoplasmic
      4.3%: peroxisomal
>> prediction for CG56868-01 is nuc (k = 23)
```

A search of the NOV62a protein against the Geneseq database, a proprietary database that contains sequences published in patents and patent publication, yielded several homologous proteins shown in Table 62D.

TABLE 62D
Geneseq Results for NOV62a

| | | NOV62a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB47567 | Protease PRTS-9 - Homo sapiens, | 1 . . . 776 | 771/776 (99%) | 0.0 |
| | 776 aa. [WO200171004-A2, 27- | 1 . . . 776 | 773/776 (99%) | |
| | SEP-2001] | | | |
| AAU77409 | Human NOV4b protein, | 1 . . . 777 | 776/780 (99%) | 0.0 |
| | homologue of ADAM proteins - | 1 . . . 779 | 776/780 (99%) | |
| | Homo sapiens, 779 aa. | | | |
| | [WO200206329-A2, 24-JAN- | | | |
| | 2002] | | | |
| AAU77408 | Human NOV4a protein, | 1 . . . 777 | 766/779 (98%) | 0.0 |
| | homologue of ADAM proteins - | 1 . . . 778 | 774/779 (99%) | |
| | Homo sapiens, 778 aa. | | | |
| | [WO200206329-A2, 24-JAN- | | | |
| | 2002] | | | |
| AAU16950 | Human novel secreted protein, | 1 . . . 677 | 664/677 (98%) | 0.0 |
| | SEQ ID 191 - Homo sapiens, 695 | 17 . . . 693 | 675/677 (99%) | |
| | aa. [WO200155441-A2, 02-AUG- | | | |
| | 2001] | | | |
| AAB64744 | Gene 16 human secreted protein | 336 . . . 710 | 353/375 (94%) | 0.0 |
| | homologous amino acid sequence | 1 . . . 375 | 364/375 (96%) | |
| | #138 - Macaca fascicularis, 375 | | | |
| | aa. [WO200077237-A1, 21-DEC- | | | |
| | 2000] | | | |

In a BLAST search of public sequence databases, the NOV62a protein was found to have homology to the proteins shown in the BLASTP data in Table 62E.

TABLE 62E
Public BLASTP Results for NOV62a

| | | NOV62a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H2U9 | ADAM 7 precursor (A disintegrin | 1 . . . 776 | 741/776 (95%) | 0.0 |
| | and metalloproteinase domain 7) | 1 . . . 754 | 751/776 (96%) | |
| | (Sperm maturation-related | | | |
| | glycoprotein GP-83) - Homo | | | |
| | sapiens (Human), 754 aa. | | | |
| Q28475 | ADAM 7 precursor (A disintegrin | 1 . . . 776 | 717/776 (92%) | 0.0 |
| | and metalloproteinase domain 7) | 1 . . . 776 | 748/776 (95%) | |
| | (Epididymal apical protein I) (EAP | | | |
| | I) - Macaca fascicularis (Crab | | | |
| | eating macaque) (Cynomolgus | | | |
| | monkey), 776 aa. | | | |
| O35227 | ADAM 7 precursor (A disintegrin | 1 . . . 774 | 523/774 (67%) | 0.0 |
| | and metalloproteinase domain 7) - | 1 . . . 771 | 638/774 (81%) | |
| | Mus musculus (Mouse), 788 aa. | | | |
| AAH43207 | Hypothetical protein - Homo | 212 . . . 736 | 524/525 (99%) | 0.0 |
| | sapiens (Human), 561 aa | 29 . . . 553 | 525/525 (99%) | |
| | (fragment). | | | |
| Q63180 | ADAM 7 precursor (A disintegrin | 1 . . . 774 | 520/774 (67%) | 0.0 |
| | and metalloproteinase domain 7) | 1 . . . 772 | 632/774 (81%) | |
| | (Epididymal apical protein I) (EAP | | | |
| | I) - Rattus norvegicus (Rat), 789 | | | |
| | aa. | | | |

PFam analysis indicates that the NOV62a protein contains the domains shown in the Table 62F.

TABLE 62F
Domain Analysis of NOV62a

| | NOV62a | Identities/ | |
|---|---|---|---|
| | Match | Similarities | Expect |
| Pfam Domain | Region | for the Matched Region | Value |
| Pep_M12B_propep | 73 . . . 188 | 38/119 (32%) | 6.7e−45 |
| | | 100/119 (84%) | |
| Reprolysin | 199 . . . 394 | 88/203 (43%) | 5.5e−104 |
| | | 173/203 (85%) | |
| disintegrin | 411 . . . 486 | 41/76 (54%) | 1.6e−25 |
| | | 53/76 (70%) | |

### Example 63

The NOV63 clone was analyzed, and the nucleotide and encoded polypeptide sequences are shown in Table 63A.

TABLE 63A
NOV63 Sequence Analysis

| | | |
|---|---|---|
| NOV63a, CG56870-01 | SEQ ID NO: 941 | 1268 bp |

| | | |
|---|---|---|
| DNA Sequence | ORF Start: ATG at 71 | ORF Stop: TAA at 1196 |

ACTTCTTTCTTTTCTGTTTCAGAGTTACTGATTTATTCTTGAGATTCCTCTACTCTCGTTATCTGACC
TCATGGATGAACTTCAGGATGTTCAGCTCACAGAGATCAAACCACTTCTAAATGATAAGAATGGTACA
AGAAACTTCCAGGACTTTGACTGTCAGGAACATGATATAGAAACAACTCATGGTGTGGTCGACGTCAC
TATAAGAGGCTTACCCAAAGGAAACAGACCAGTTATACTAACATATCATGACATTGGCCTCAACCGTA
AATCCTGTTTCAATGCATTCTTTAACTTTGAGGATATGCAAGAGATCACCCAGCACTTTGCTGTCTGT
CATGTGGATGCCCCAGGCCAGCAGGAAGGTGCACCCTCTTTCCCAACAGGGTATCAGTACCCCACAAT
GGATGAGCTGGCTGAAATGCTGCCTCCTGTTCTTACCCACCTAAGCCTGAAAAGCATCATTGGAATTG
GAGTTGGAGCTGGAGCTTACATCCTCAGCAGATTTGCACTCAACCATCCAGAGCTTGTGGAAGGCCTT
GTGCTCATTAATGTTGACCCTTGCGCTAAAGGCTGGATTGACTGGGCAGCTTCCAAACTCTCTGGCCT
GACAACCAATGTTGTGGACATTATTTTGGCTCATCACTTTGGGCAGGAAGAGTTACAGGCCAACCTGG
ACCTGATCCAAACCTACAGAATGCATATTGCCCAAGACATCAACCAAGACAACCTGCAGCTCTTCTTG
AATTCCTACAATGGGCGCAGAGACCTGGAGATCGAAAGACCCATACTGGGCCAAAATGATAACAAATC
AAAAACATTAAAGTGTTCTACTTTACTGGTGGTAGGGGACAATTCGCCTGCAGTTGAGGCTGTGGTCG
AATGCAATTCCCGCCTGAACCCTATAAATACAACTTTGCTAAAGATGGCGGACTGTGGGGGACTGCCC
CAGGTAGTTCAGCCTGGGAAGCTCACCGAGGCCTTCAAGTACTTTTTGCAGGGAATGGGCTACGTCCC
GTCTGCCAGCATGACTCGGCTCGCCCGATCACGAACCCACTCAACCTCGAGTAGCCTCGGCTCTGGAG
AAAGTCCCTTCAGCCGGTCTGTCACCAGCAATCAGTCAGATGGAACTCAAGAATCCTGTGAGTCCCCT
GATGTCCTGGACAGACACCAGACCATGGAGGTGTCCTGCTAAGCAGATGCTCCTCCCCTGGACCATTG
CAAGTCCATCCTTCAAATGACCACTCCATAATATAACATTTCAT

NOV63a, CG56870-01

| | | | |
|---|---|---|---|
| Protein Sequence | SEQ ID NO: 942 | 375 aa | MW at 41413.3kD |

MDELQDVQLTEIKPLLNDKNGTRNFQDFDCQEHDIETTHGVVHVTIRGLPKGNRPVILTYHDIGLNRK
SCFNAFFNFEDMQEITQHFAVCHVDAPGQQEGAPSFPTGYQYPTMDELAEMLPPVLTHLSLKSIIGIG
VGAGAYILSRFALNHPELVEGLVLINVDPCAKGWIDWAASKLSGLTTNVVDIILAHHFGQEELQANLD
LIQTYRMHIAQDINQDNLQLFLNSYNGRRDLEIERPILGQNDNKSKTLKCSTLLVVGDNSPAVEAVVE
CNSRLNPINTTLLKMADCGGLPQVVQPGKLTEAFKYFLQGMGYVPSASMTRLARSRTHSTSSSLGSGE
SPFSRSVTSNQSDGTQESCESPDVLDRHQTMEVSC

| | | |
|---|---|---|
| NOV63b, CG56870-06 | SEQ ID NO: 943 | 1041 bp |

| | | |
|---|---|---|
| DNA Sequence | ORF Start: ATG at 120 | ORF Stop: TAA at 969 |

ACTTCTTTCTTTTCTGTTTCAGAGTTACTGATTTATTCTTGAGATTCCTCTACTCTCGTTATCTGACC
TCATGGATGAACTTCAGGATGTTCAGCTCACAGAGATCAAACCACTTCTAAATGATAAGAATGGTACA
AGAAACTTCCAGGACTTTGACTGTCAGGTATCAGTACCCCACAATGGATGAGCTGGCTGAAATGCTGC
CTCCTGTTCTTACCCACCTAAGCCTGAAAAGCATCATTGGAATTGGAGTTGGAGCTGGAGCTTACATC
CTCAGCAGATTTGCACTCAACCATCCAGAGCTTGTGGAAGGCCTTGTGCTCATTAATGTTGACCCTTG
CGCTAAAGGCTGGATTGACTGGGCAGCTTCCAAACTCTCTGGCCTGACAACCAATGTTGTGGACATTA
TTTTGGCTCATCACTTTGGGCAGGAAGAGTTACAGGCCAACCTGGACCTGATCCAAACCTACAGAATG
CATATTGCCCAAGACATCAACCAAGACAACCTGCAGCTCTTCTTGAATTCCTACAATGGGCGCAGAGA
CCTGGAGATCGAAAGACCCATACTGGGCCAAAATGATAACAAATCAAAAACATTAAAGTGTTCTACTT
TACTGGTGGTAGGGGACAATTCGCCTGCAGTTGAGGCTGTGGTCGAATGCAATTCCCGCCTGAACCCT
ATAAATACAACTTTGCTAAAGATGGCGGACTGTGGGGGACTGCCCCAGGTAGTTCAGCCTGGGAAGCT
CACCGAGGCCTTCAAGTACTTTTTGCAGGGAATGGGCTACGTCCCGTCTGCCAGCATGACTCGGCTCG
CCCGATCACGAACCCACTCAACCTCGAGTAGCCTCGGCTCTGGAGAAAGTCCCTTCAGCCGGTCTGTC
ACCAGCAATCAGTCAGATGGAACTCAAGAATCCTGTGAGTCCCCTGATGTCCTGGACAGACACCAGAC
CATGGAGGTGTCCTGCTAAGCAGATGCTCCTCCCCTGGACCATTGCAAGTCCATCCTTCAAATGACCA
CTCCATAATATAACATTTCAT

NOV63b, CG56870-06

| | | | |
|---|---|---|---|
| Protein Sequence | SEQ ID NO: 944 | 283 aa | MW at 31109.1kD |

MIRMVQETSRTLTVRYQYPTMDELAEMLPPVLTHLSLKSIIGIGVGAGAYILSRFALNHPELVEGLVL
INVDPCAKGWIDWAASKLSGLTTNVVDIILAHHFGQEELQANLDLIQTYRMHIAQDINQDNLQLFLNS
YNGRRDLEIERPILGQNDNKSKTLKCSTLLVVGDNSPAVEAVVECNSRLNPINTTLLKMADCGGLPQV
VQPGKLTEAFKYFLQGMGYVPSASMTRLARSRTHSTSSSLGSGESPFSRSVTSNQSDGTQESCESPDV
LDRHQTMEVSC

| | | |
|---|---|---|
| NOV63c, 276585681 | SEQ ID NO: 945 | 994 bp |

| | | |
|---|---|---|
| DNA Sequence | ORF Start: at 2 | ORF Stop: end of sequence |

CACCAAGCTTACAAGAAACTTCCAGGACTTTGACTGTCAGGAACATGATATAGAAACAACTCATGGTG
TGGTCCACGTCACTATAAGAGGCTTACCCAAAGGAAACAGACCAGTTATACTAACATATCATGACATT
GGCCTCAACCATAAATCCTGTTTCAATGCATTCTTTAACTTTGAGGATATGCAAGAGATCACCCAGCA
CTTTGCTGTCTGTCATGTGGATGCCCCAGGCCAGCAGGAAGGTGCACCCTCTTTCCCAACAGGGTATC
AGTACCCCACAATGGATGAGCTGGCTGAAGTGCTGCCTCCTGTTCTTACCCACCTAAGCCTGAAAAGC
ATCATTGGAATTGGAGTTGGAGCTGGAGCTTACATCCTCAGCAGATTTGCACTCAACCATCCAGAGCT
TGTGGAAGGCCTTGTGCTCATTAATGTTGACCCTTGCGCTAAAGGCTGGATTGACTGGGCAGCTTCCA
AACTCTCTGGCCTGACAACCAATGTTGTGGACATTATTTTGGCTCATCACTTTGGGCAGGAAGAGTTA
CAGGCCAACCTGGACCTGATCCAAACCTACAGAATGCATATTGCCCAAGACATCAACCAAGACAACCT
GCAGCTCTTCTTGAATTCCTACAATGGACGCAGAGACCTGGAGATCGAAAGACCCATACTGGGCCAAA
ATGATAACAAATCAAAAACATTAAAGTGTTCTACTTTACTGGTGGTAGGGGACAATTCGCCTGCAGTT
GAGGCTGTGGTCGAATGCAATTCCCGCCTGAACCCTATAAATACAACTTTGCTAAAGATGGCGGACTG
TGGGGGACTGCCCCAGGTAGTTCAGCCTGGGAAGCTCACCGAGGCCTTCAAGTACTTTTTGCAGGGAA
TGGGCTACATACCATCTGCCAGCATGACTCGGCTCGCCCGATCACGAACCCACTCAACCTCGAGTAGC
CTCGGCTCTGGAGAAAGTCCCTTCAGCCGGTCTGTCGACGGC

NOV63c, 276585681

| | | | |
|---|---|---|---|
| Protein Sequence | SEQ ID NO: 946 | 331 aa | MW at 36428.9kD |

TKLTRNFQDFDCQEHDIETTHGVVHVTIRGLPKGNRPVILTYHDIGLNHKSCFNAFFNFEDMQEITQH
FAVCHVDAPGQQEGAPSFPTGYQYPTNDELAEVLPPVLTHLSLKSIIGIGVGAGAYILSRFALNHPEL
VEGLVLINVDPCAKGWIDWAASKLSGLTTNVVDIILAHHFGQEELQANLDLIQTYRMHIAQDINQDNL
QLFLNSYNGRRDLEIERPILGQNDNKSKTLKCSTLLVVGDNSPAVEAVVECNSRLNPINTTLLKMADC
GGLPQVVQPGKLTEAFKYFLQGMGYIPSASMTRLARSRTHSTSSSLGSGESPFSRSVDG

| | | |
|---|---|---|
| NOV63d, CG56870-02 | SEQ ID NO: 947 | 1175 bp |

| | | |
|---|---|---|
| DNA Sequence | ORF Start: ATG at 16 | ORF Stop: TAA at 1141 |

TCGTTATCTGACCTCATGGATGAACTTCAGGATGTTCAGCTCACAGAGATCAAACCACTTCTAAATGA
TAAGAATGGTACAAGAAACTTCCAGGACTTTGACTGTCAGGAACATGATATAGAAACAACTCATGGTG
TGGTCCACGTCACTATAAGAGGCTTACCCAAAGGAAACAGACCAGTTATACTAACATATCATGACATT
GGCCTCAACCATAAATCCTGTTTCAATGCATTCTTTAACTTTGAGGATATGCAAGAGATCACCCAGCA
CTTTGCTGTCTGTCATGTGGATGCCCCAGGCCAGCAGGAAGGTGCACCCTCTTTCCCAACAGGGTATC
AGTACCCCACAATGGATGAGCTGGCTGAAGTGCTGCCTCCTGTTCTTACCCACCTAAGCCTGAAAAGC
ATCATTGGAATTGGAGTTGGAGCTGGAGCTTACATCCTCAGCAGATTTGCACTCAACCATCCAGAGCT
TGTGGAAGGCCTTGTGCTCATTAATGTTGACCCTTGCGCTAAAGGCTGGATTGACTGGGCAGCTTCCA
AACTCTCTGGCCTGACAACCAATGTTGTGGACATTATTTTGGCTCATCACTTTGGGCAGGAAGAGTTA
CAGGCCAACCTGGACCTGATCCAAACCTACAGAATGCATATTGCCCAAGACATCAACCAAGACAACCT
GCAGCTCTTCTTGAATTCCTACAATGGACGCAGAGACCTGGAGATCGAAAGACCCATACTGGGCCAAA
ATGATAACAAATCAAAAACATTAAAGTGTTCTACTTTACTGGTGGTAGGGGACAATTCGCCTGCAGTT
GAGGCTGTGGTCGAATGCAATTCCCGCCTGAACCCTATAAATACAACTTTGCTAAAGATGGCGGACTG
TGGGGGACTGCCCCAGGTAGTTCAGCCTGGGAAGCTCACCGAGGCCTTCAAGTACTTTTTGCAGGGAA
TGGGCTACATACCATCTGCCAGCATGACTCGGCTCGCCCGATCACGAACCCACTCAACCTCGAGTAGC
CTCGGCTCTGGAGAAAGTCCCTTCAGCCGGTCTGTCACCAGCAATCAGTCACATGGAACTCAAGAATC
CTGTGAGTCCCCTGATGTCCTGGACAGACACCAGACCATGGAGGTGTCCTGCTAAGCAGATGCTCCTC
CCCTGGACCATTGCAAGTC

NOV63d, CG56870-02

| | | | |
|---|---|---|---|
| Protein Sequence | SEQ ID NO: 948 | 375 aa | MW at 41376.2kD |

MDELQDVQLTEIKPLLNDKNGTRNFQDFDCQEHDIETTHGVVHVTIRGLPKGNRPVILTYHDIGLNHK
SCFNAFFNFEDMQEITQHFAVCHVDAPGQQEGAPSFPTGYQYPTMDELAEVLPPVLTHLSLKSIIGIG
VGAGAYILSRFALNHPELVEGLVLINVDPCAKGWIDWAASKLSGLTTNVVDIILAHHFGQEELQANLD
LIQTYRNHIAQDINQDNLQLFLNSYNGRRNLEIERPILGQNDNKSKTLKCSTLLVVGDNSPAVEAVVE
CNSRLNPINTTLLKMADCGGLPQVVQPGKLTEAFKYFLQGMGYIPSASMTRLARSRTHSTSSSLGSGE
SPFSRSVTSNQSDGTQESCESPDVLDRHQTMEVSC

| | | |
|---|---|---|
| NOV63e, CG56870-03 | SEQ ID NO: 949 | 1232 bp |

| | | |
|---|---|---|
| DNA Sequence | ORF Start: ATG at 71 | ORF Stop: TAA at 1160 |

ACTTCTTTCTTTTCTGTTTCAGAGTTACTGATTTATTCCTGAGATTCCTCTACTCTCGTTATCTGACC
TCATGGATGAACTTCAGGATGTTCAGCTCACAGAGATCAAACCACTTCTAAATGATAAGGAACATGAT
ATAGAAACAACTCATGGTGTGGTCCACGTCACTATAAGAGGCTTACCCAAAGGAAACAGACCAGTTAT
ACTAACATATCATGACATTGGCCTCAACCATAAATCCTGTTTCAATGCATTCTTTAACTTTGAGGATA
TGCAAGAGATCACCCAGCACTTTGCTGTCTGTCATGTGGATGCCCCAGGCCAGCAGGAAGGTGCACCC
TCTTTCCCAACAGGGTATCAGTACCCCACAATGGATGAGCTGGCTGAAATGCTGCCTCCTGTTCTTAC
CCACCTAAGCCTGAAAAGCATCATTGGAATTGGAGTTGGAGCTGGAGCTTACATCCTCAGCAGATTTG
CACTCAACCATCCAGAGCTTGTGGAAGGCCTTGTGCTCATTAATGTTGACCCTTGCGCTAAAGGCTGG
ATTGACTGGGCAGCTTCCAAACTCTCTGGCCTGACAACCAATGTTGTGGACATTATTTTGGCTCATCA
CTTTGGGCAGGAAGAGTTACAGGCCAACCTGGACCTGATCCAAACCTACAGAATGCATATTGCCCAAG
ACATCAACCAAGACAACCTGCAGCTCTTCTTGAATTCCTACAATGGGCGCAGAGACCTGGAGATCGAA
AGACCCATACTGGGCCAAAATGATAACAAATCAAAAACATTAAAGTGTTCTACTTTACTGGTGGTAGG
GGACAATTCGCCTGCAGTTGAGGCTGTGGTCGAATGCAATTCCCGCCTGAACCCTATAAATACAACTT
TGCTAAAGATGGCGGACTGTGGGAAACTGCCCCAGGTAGTTCAGCCTGGGAAGCTCACCGAGGCCTTC
AAGTACTTTTTGCAGGGAATGGGCTACGTCCCGTCTGCCAGCATGACTCGGCTCGCCCGATCACGAAC
CCACTCAACCTCGAGTAGCCTCGGCTCTGGAGAAAGTCCCTTCAGCCGGTCTGTCACCAGCAATCAGT
CAGATGGAACTCAAGAATCCTGTGAGTCCCCTGATGTCCTGGACAGACACCAGACCATGGAGGTGTCC
TGCTAAGCAGATGCTCCTCCCCTGGACCATTGCAAGTCCATCCTTCAAATGACCACTCCATAATATAA
CATTTCAT

NOV63e, CG56870-03

| | | | |
|---|---|---|---|
| Protein Sequence | SEQ ID NO: 950 | 363 aa | MW at 39967.8kD |

MDELQDVQLTEIKPLLNDKEHDIETTHGVVHVTIRGLPKGNRPVILTYHDIGLNHKSCFNAFFNFEDM
QEITQHFAVCHVDAPGQQEGAPSFPTGYQYPTMDELAEMLPPVLTHLSLKSIIGIGVGAGAYILSRFA
LNHPELVEGLVLINVDPCAKGWIDWAASKLSGLTTNVVDIILAHHFGQEELQANLDLIQTYRMHIAQD
INQDNLQLFLNSYNGRRDLEIERPILGQNDNKSKTLKCSTLLVVGDNSPAVEAVVECNSRLNPINTTL
LKMADCGGLPQVVQPGKLTEAFKYFLQGMGYVPSASMTRLATSRTHSTSSSLGSGESPFSRSVTSNQS
DGTQESCESPDVLDRHQTMEVSC

| | | |
|---|---|---|
| NOV63f, CG56870-04 | SEQ ID NO: 951 | 1220 bp |

| | | |
|---|---|---|
| DNA Sequence | ORF Start: ATG at 71 | ORF Stop: TAA at 1148 |

ACTTCTTTCTTTTCTGTTTCAGAGTTACTGATTTATTCTTGAGATTCCTCTACTCTCGTTATCTGACC
TCATGGATGAACTTCAGGATGTTCAGCTCACAGAGATCAAACCACTTCTAAATGATAAGAATGGTACA
AGAAACTTCCAGGACTTTGACTGTCAGGAACATGATATAGAAACAACTCATGGTGTGGTCCACGTCAC
TATAAGAGGCTTACCCAAAGGAAACAGACCAGTTATACTAACATATCATGACATTGGCCTCAACCGTA
AATCCTGTTTCAATGCATTCTTTAACTTTGAGGATATGCAAGAGATCACCCAGCACTTTGCTGTCTGT
CATGTGGATGCCCCAGGCCAGCAGGAAGGTGCACCCTCTTTCCCAACAGGGTATCAGTACCCCACAAT
GGATGAGCTGGCTGAAATGCTGCCTCCTGTTCTTACCCACCTAAGCCTGAAAAGCATCATTGGAATTG
GAGTTGGAGCTGGAGCTTACATCCTCAGCAGATTTGCACTCAACCATCCAGAGCTTGTGGAAGGCCTT
GTGCTCATTAATGTTGACCCTTGCGCTAAAGGCTGGATTGACTGGGCAGCTTCCAAACTCTCTGGCCT
GACAACCAATGTTGTGGACATTATTTTGGCTCATCACTTTGGGCAGGAAGAGTTACAGGCCAACCTGG
ACCTGATCCAAACCTACAGAATGCATATTGCCCAAGACATCAACCAAGACAACCTGCAGCTCTTCTTG
AATTCCTACAATGGACGCAGAGACCTGGAGATCGAAAGACCCATACTGGGCCAAAATGATAACAAATC
AAAAACATTAAAGTGTTCTACTTTACTGGTGGTAGGGGACAATTCGCCTGCAGTTGAGGCTGTGATGG
CGGACTGTGGGGGACTGCCCCAGGTAGTTCAGCCTGGGAAGTTCACCGAGGCCTTCAAGTACTTTTTG
CAGGGAATGGGCTACACACCATCTGCCAGCATGACTCGGCTCGCCCGATCACGAACCCACTCAACCTC
GAGTAGCCTCGGCTCTGGAGAAAGTCCCTTCAGCCGGTCTGTCACCAGCAATCAGTCAGATGGAACTC
AAGAATCCTGTGAGTCCCCTGATGTCCTGGACAGACACCAGACCATGGAGGTGTCCTGCTAAGCAGAT
GCTCCTCCCCTGGACCATTGCAAGTCCATCCTTCAAATGACCACTCCATAATATAACATTTCAT

NOV63f, CG56870-04

| | | | |
|---|---|---|---|
| Protein Sequence | SEQ ID NO: 952 | 359 aa | MW at 39652.2kD |

MDELQDVQLTEIKPLLNDKNGTRNFQDFDCQEHDIETTHGVVHVTIRGLPKGNRPVILTYHDIGLNRK
SCFNAFFNFEDMQEITQHFAVCHVDAPGQQEGAPSFPTGYQYPTMDELAEMLPPVLTHLSLKSIIGIG
VGAGAYILSRFALNHPELVEGLVLINVDPCAKGWIDWAASKLSGLTTNVVDIILAHHFGQEELQANLD
LIQTYRNHIAQDINQDNLQLFLNSYNGRRDLEIERPILGQNDNKSKTLKCSTLLVVGDNSPAVEAVMA
DCGGLPQVVQPGKFTEAFKYFLQGMGYTPSASMTRLARSRTHSTSSSLGSGESPFSRSVTSNQSDGTQ
ESCESPDVLDRHQTMEVSC

| | | |
|---|---|---|
| NOV63g, CG56870-05 | SEQ ID NO: 953 | 1970 bp |

| | | |
|---|---|---|
| DNA Sequence | ORF Start: ATG at 1 | ORF Stop: TAA at 898 |

ATGGATGAACTTCAGGATGTTCAGCTCACAGAGATCAAACCACTTCTAAATGATAAGAATGGTACAAG
AAACTTCCAGGACTTTGACTGTCAGTATCAGTACCCCACAATGGATGAGCTGGCTGAAATGCTGCCTC
CTGTTCTTACCCACCTAAGCCTGAAAAGCATCATTGGAATTGGAGTTGGAGCTGGAGCTTACATCCTC
AGCAGATTTGCACTCAACCATCCAGAGCTTGTGGAAGGCCTTGTGCTCATTAATGTTGACCCTTGCGC
TAAAGGCTGGATTGACTGGGCAGCTTCCAAACTCTCTGGCCTGACAACCAATGTTGTGGACATTATTT
TGGCTCATCACTTTGGGCAGGAAGAGTTACAGGCCAACCTGGACCTGATCCAAACCTACAGAATGCAT
ATTGCCCAAGACATCAACCAAGACAACCTGCAGCTCTTCTTGAATTCCTACAATGGGCGCAGAGACCT
GGAGATCGAAAGACCCATACTGGGCCAAAATGATAACAAATCAAAAACATTAAAGTGTTCTACTTTAC
TGGTGGTAGGGGACAATTCGCCTGCAGTTGAGGCTGTGGTCGAATGCAATTCCCGCCTGAACCCTATA
AATACAACTTTGCTAAAGATGGCGGACTGTGGGGGACTGCCCCAGGTAGTTCAGCCTGGGAAGCTCAC
CGAGGCCTTCAAGTACTTTTTGCAGGGAATGGGCTACGTCCCGTCTGCCAGCATGACTCGGCTCGCCC
GATCACGAACCCACTCAACCTCGAGTAGCCTCGGCTCTGGAGAAAGTCCCTTCAGCCGGTCTGTCACC
AGCAATCAGTCAGATGGAACTCAAGAATCCTGTGAGTCCCCTGATGTCCTGGACAGACACCAGACCAT
GGAGGTGTCCTGCTAAGCAGATGCTCCTCCCCTGGACCATTGCAAGTCCATCCTTCAAATGACCACTC
CATAATATAACATTTCAT

NOV63g, CG56870-05

| | | | |
|---|---|---|---|
| Protein Sequence | SEQ ID NO: 954 | 299 aa | MW at 32956.9kD |

MDELQDVQLTEIKPLLNDKNGTRNFQDFDCQYQYPTMDELAEMLPPVLTHLSLKSIIGIGVGAGAYIL
SRFALNHPELVEGLVLINVDPCAKGWIDWAASKLSGLTTNVVDIILAHHFGQEELQANLDLIQTYRMH
IAQDINQDNLQLFLNSYNGRRDLEIERPILGQNDNKSKTLKCSTLLVVGDNSPAVEAVVECNSRLNPI
NTTLLKMADCGGLPQVVQPGKLTEAFKYFLQGMGYVPSASMTRLARSRTHSTSSSLGSGESPFSRSVT
SNQSDGTQESCESPDVLDRHQTMEVSC

A ClustalW comparison of the above protein sequences yields the following sequence alignment shown in Table 63B.

TABLE 63B
Comparison of the NOV63 protein sequences.

| | |
|---|---|
| NOV63a | MDELQDVQLTEIKPLLNDKNGTRNFQDFDCQEHDIETTHGVVHVTIRGLPKGNRPVILTY |
| NOV63b | ------------------------------------------------------------ |
| NOV63c | ------------------TKLTRNFQDFDCQEHDIETTHGVVHVTIRGLPKGNRPVILTY |
| NOV63d | MDELQDVQLTEIKPLLNDKNGTRNFQDFDCQEHDIETTHGVVHVTIRGLPKGNRPVILTY |
| NOV63e | ------------MDELQDVQLTEIKPLLNDKEHDIETTHGVVHVTIRGLPKGNRPVILTY |
| NOV63f | MDELQDVQLTEIKPLLNDKNGTRNFQDFDCQEHDIETTHGVVHVTIRGLPKGNRPVILTY |
| NOV63g | ------------------------------------------------------------ |
| NOV63a | HDIGLNRKSCFNAFFNFEDMQEITQHFAVCHVDAPGQQEGAPSFPTGYQYPTMDELAEML |
| NOV63b | ---------------------------MIRMVQETS-----RTLTVRYQYPTMDELAEML |
| NOV63c | HDIGLNHKSCFNAFFNFEDMQEITQHFAVCHVDAPGQQEGAPSFPTGYQYPTMDELAEVL |
| NOV63d | HDIGLNHKSCFNAFFNFEDMQEITQHFAVCHVDAPGQQEGAPSFPTGYQYPTMDELAEVL |
| NOV63e | HDIGLNHKSCFNAFFNFEDMQEITQHFAVCHVDAPGQQEGAPSFPTGYQYPTMDELAEVL |
| NOV63f | HDIGLNRKSCFNAFFNFEDMQEITQHFAVCHVDAPGQQEGAPSFPTGYQYPTMDELAEVL |
| NOV63g | ----------------MDELQDVQLTEIKPLLNDKNGTRNFQDFDCQYQYPTMDELAEVL |
| NOV63a | PPVLTHLSLKSIIGIGVGAGAYILSRFALNHPELVEGLVLINVDPCAKGWIDWAASKLSG |
| NOV63b | PPVLTHLSLKSIIGIGVGAGAYILSRFALNHPELVEGLVLINVDPCAKGWIDWAASKLSG |
| NOV63c | PPVLTHLSLKSIIGIGVGAGAYILSRFALNHPELVEGLVLINVDPCAKGWIDWAASKLSG |
| NOV63d | PPVLTHLSLKSIIGIGVGAGAYILSRFALNHPELVEGLVLINVDPCAKGWIDWAASKLSG |
| NOV63e | PPVLTHLSLKSIIGIGVGAGAYILSRFALNHPELVEGLVLINVDPCAKGWIDWAASKLSG |
| NOV63f | PPVLTHLSLKSIIGIGVGAGAYILSRFALNHPELVEGLVLINVDPCAKGWIDWAASKLSG |
| NOV63g | PPVLTHLSLKSIIGIGVGAGAYILSRFALNHPELVEGLVLINVDPCAKGWIDWAASKLSG |
| NOV63a | LTTNVVDIILAHHFGQEELQANLDLIQTYRMHIAQDINQDNLQLFLNSYNGRRDLEIERP |
| NOV63b | LTTNVVDIILAHHFGQEELQANLDLIQTYRMHIAQDINQDNLQLFLNSYNGRRDLEIERP |
| NOV63c | LTTNVVDIILAHHFGQEELQANLDLIQTYRMHIAQDINQDNLQLFLNSYNGRRDLEIERP |
| NOV63d | LTTNVVDIILAHHFGQEELQANLDLIQTYRMHIAQDINQDNLQLFLNSYNGRRDLEIERP |
| NOV63e | LTTNVVDIILAHHFGQEELQANLDLIQTYRMHIAQDINQDNLQLFLNSYNGRRDLEIERP |
| NOV63f | LTTNVVDIILAHHFGQEELQANLDLIQTYRMHIAQDINQDNLQLFLNSYNGRRDLEIERP |
| NOV63g | LTTNVVDIILAHHFGQEELQANLDLIQTYRMHIAQDINQDNLQLFLNSYNGRRDLEIERP |
| NOV63a | ILGQNDNKSKTLKCSTLLVVGDNSPAVEAVVECNSRLNPINTTLLKMADCGGLPQVVQPG |
| NOV63b | ILGQNDNKSKTLKCSTLLVVGDNSPAVEAVVECNSRLNPINTTLLKMADCGGLPQVVQPG |
| NOV63c | ILGQNDNKSKTLKCSTLLVVGDNSPAVEAVVECNSRLNPINTTLLKMADCGGLPQVVQPG |
| NOV63d | ILGQNDNKSKTLKCSTLLVVGDNSPAVEAVVECNSRLNPINTTLLKMADCGGLPQVVQPG |
| NOV63e | ILGQNDNKSKTLKCSTLLVVGDNSPAVEAVVECNSRLNPINTTLLKMADCGGLPQVVQPG |
| NOV63f | ILGQNDNKSKTLKCSTLLVVGDNSPAVEAV----------------MADCGGLPQVVQPG |
| NOV63g | ILGQNDNKSKTLKCSTLLVVGDNSPAVEAVVECNSRLNPINTTLLKMADCGGLPQVVQPG |
| NOV63a | KLTEAFKYFLQGMGYVPSASMTRLARSRTHSTSSSLGSGESPFSRSVTSNQSDGTQESCE |
| NOV63b | KLTEAFKYFLQGMGYVPSASMTRLARSRTHSTSSSLGSGESPFSRSVTSNQSDGTQESCE |
| NOV63c | KLTEAFKYFLQGMGYVPSASMTRLARSRTHSTSSSLGSGESPFSRSVDG----------- |
| NOV63d | KLTEAFKYFLQGMGYVPSASMTRLARSRTHSTSSSLGSGESPFSRSVTSNQSDGTQESCE |
| NOV63e | KLTEAFKYFLQGMGYVPSASMTRLARSRTHSTSSSLGSGESPFSRSVTSNQSDGTQESCE |
| NOV63f | KLTEAFKYFLQGMGYVPSASMTRLARSRTHSTSSSLGSGESPFSRSVTSNQSDGTQESCE |
| NOV63g | KLTEAFKYFLQGMGYVPSASMTRLARSRTHSTSSSLGSGESPFSRSVTSNQSDGTQESCE |
| NOV63a | SPDVLDRHQTMEVSC |
| NOV63b | SPDVLDRHQTMEVSC |
| NOV63c | --------------- |
| NOV63d | SPDVLDRHQTMEVSC |
| NOV63e | SPDVLDRHQTMEVSC |
| NOV63f | SPDVLDRHQTMEVSC |
| NOV63g | SPDVLDRHQTMEVSC |
| NOV63a | (SEQ ID NO: 942) |
| NOV63b | (SEQ ID NO: 944) |
| NOV63c | (SEQ ID NO: 946) |
| NOV63d | (SEQ ID NO: 948) |
| NOV63e | (SEQ ID NO: 950) |
| NOV63f | (SEQ ID NO: 952) |
| NOV63g | (SEQ ID NO: 954) |

Further analysis of the NOV63a protein yielded the following properties shown in Table 63C.

TABLE 63C
Protein Sequence Properties NOV63a

| | |
|---|---|
| SignalP analysis: | No Known Signal Sequence Indicated |
| PSORT II analysis: | |

```
PSG: a new signal peptide prediction method
    N-region: length 11; pos.chg 0; neg.chg 4
    H-region: length 1; peak value 0.00
    PSG score: −4.40
GvH: von Heijne's method for signal seq. recognition
    GvH score (threshold: −2.1): −14.03
    possible cleavage site: between 48 and 49
>>> Seems to have no N-terminal signal peptide
ALOM: Klein et al's method for TM region allocation
    Init position for calculation: 1
    Tentative number of TMS(s) for the threshold 0.5: 1
    Number of TMS(s) for threshold 0.5: 0
    PERIPHERAL Likelihood = 4.67 (at 174)
    ALOM score:  0.47 (number of TMSs: 0)
MITDISC: discrimination of mitochondrial targeting seq
```

| | | | |
|---|---|---|---|
| R content: | 0 | Hyd Moment(75): | 8.01 |
| Hyd Moment(95): | 7.59 | G content: | 0 |
| D/E content: | 2 | S/T content: | 0 |
| Score: −6.61 | | | |

```
Gavel: prediction of cleavage sites for mitochondrial preseq
    cleavage site motif not found
NUCDISC: discrimination of nuclear localization signals
    pat4: none
    pat7: none
    bipartite: none
    content of basic residues: 7.5%
    NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: cytoplasmic
    Reliability: 55.5
COIL: Lupas's algorithm to detect coiled-coil regions
    total: 0 residues
Final Results (k = 9/23):
    39.1%: cytoplasmic
    39.1%: nuclear
    13.0%: mitochondrial
      4.3%: Golgi
      4.3%: endoplasmic reticulum
>> prediction for CG56870-01 is cyt (k = 23)
```

A search of the NOV63a protein against the Geneseq database, a proprietary database that contains sequences published in patents and patent publication, yielded several homologous proteins shown in Table 63D.

TABLE 63D
Geneseq Results for NOV63a

| | | NOV63a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB94494 | Human protein sequence SEQ ID | 1 . . . 375 | 360/375 (96%) | 0.0 |
| | NO: 15186 - Homo sapiens, 363 | 1 . . . 363 | 361/375 (96%) | |
| | aa. [EP1074617-A2, 07-FEB- | | | |
| | 2001] | | | |
| AAG64392 | Human reducing agent and | 1 . . . 375 | 360/375 (96%) | 0.0 |
| | tunicamycin-responsive protein | 1 . . . 363 | 361/375 (96%) | |
| | 40 - Homo sapiens, 363 aa. | | | |
| | [WO200155375-A1, 02-AUG- | | | |
| | 2001] | | | |
| AAM94019 | Human stomach cancer expressed | 1 . . . 375 | 360/375 (96%) | 0.0 |
| | polypeptide SEQ ID NO 108 - | 1 . . . 363 | 361/375 (96%) | |
| | Homo sapiens, 363 aa. | | | |
| | [WO200109317-A1, 08-FEB- | | | |
| | 2001] | | | |
| ABG10563 | Novel human diagnostic protein | 68 . . . 374 | 283/323 (87%) | e−155 |
| | #10554 - Homo sapiens, 382 aa. | 1 . . . 307 | 287/323 (88%) | |
| | [WO200175067-A2, 11-OCT- | | | |
| | 2001] | | | |
| AAU31598 | Novel human secreted protein | 68 . . . 374 | 282/323 (87%) | e−154 |
| | #2089 - Homo sapiens, 395 aa. | 1 . . . 307 | 286/323 (88%) | |
| | [WO200179449-A2, 25-OCT- | | | |
| | 2001] | | | |

In a BLAST search of public sequence databases, the NOV63a protein was found to have homology to the proteins shown in the BLASTP data in Table 63E.

TABLE 63E
Public BLASTP Results for NOV63a

| | | NOV63a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UGV2 | NDRG3 protein - Homo sapiens | 1 . . . 375 | 373/375 (99%) | 0.0 |
| | (Human), 375 aa. | 1 . . . 375 | 374/375 (99%) | |
| Q9QYF9 | NDRG3 protein (Ndr3 protein) - | 1 . . . 375 | 358/375 (95%) | 0.0 |
| | Mus musculus (Mouse), 375 aa. | 1 . . . 375 | 368/375 (97%) | |
| Q8VCV2 | Similar to N-myc downstream | 1 . . . 375 | 359/388 (92%) | 0.0 |
| | regulated 3 - Mus musculus | 1 . . . 388 | 368/388 (94%) | |
| | (Mouse), 388 aa. | | | |
| Q96SM2 | Hypothetical protein FLJ14759 - | 1 . . . 375 | 360/375 (96%) | 0.0 |
| | Homo sapiens (Human), 363 aa. | 1 . . . 363 | 361/375 (96%) | |
| AAH44139 | Similar to N-myc downstream | 1 . . . 375 | 290/375 (77%) | e−169 |
| | regulated 3 - Brachydanio rerio | 2 . . . 371 | 324/375 (86%) | |
| | (Zebrafish) (Danio rerio), 371 aa. | | | |

PFam analysis indicates that the NOV63a protein contains the domains shown in the Table 63F.

TABLE 63F
Domain Analysis of NOV63a

| | NOV63a | Identities/ | |
|---|---|---|---|
| | Match | Similarities | |
| Pfam Domain | Region | for the Matched Region | Expect Value |
| abhydrolase | 87 . . . 307 | 46/237 (19%) | 0.0014 |
| | | 142/237 (60%) | |
| Ndr | 32 . . . 317 | 181/301 (60%) | 5.6e−186 |
| | | 275/301 (91%) | |

### Example 64

The NOV64 clone was analyzed, and the nucleotide and encoded polypeptide sequences are shown in Table 64A.

TABLE 64A
NOV64 Sequence Analysis

| | | |
|---|---|---|
| NOV64a, CG57109-01 | SEQ ID NO: 955 | 2536 bp |

| | | |
|---|---|---|
| DNA Sequence | ORF Start: ATG at 150 | ORF Stop: TAG at 2094 |

GGGACACTGACATGGACTGAAGGAGTAGAAAAGAAGCCTTGGGCTCTCCCAGATGGAAGAATGACCGT
GTGAGGAAACTGTTTAACCTCAAGGGCAGGGAAATCAGGAGCGTCTCTGATTTCTTCAGGGAAGGGGA
TGCTTTCATAGCTATGGGCAAAGAACCACTGACACTGAAGAGCATTCAGGTGGCTGTAGAAGAACTGT
ACCCCAACAAAGCCCGGGCCCTGACACTGGCCCAGCACAGCCGTGCCCCTTCTCCAAGGCTGAGGAGC
AGGCTGTTTAGCAAGGCTCTGAAAGGAGACCACCGCTGTGGGGAGACCGAGACCCCCAAGAGCTGCAG
CGAAGTTGCAGGATGCAAGGCAGCCATGAGGCACCAGGGGAAGATCCCCGAGGAGCTTTCACTAGATG
ACAGAGCGAGGACCCAGAAGAAGTGGGGGAGGGGGAAATGGGAGCCAGAACCCAGTAGCAAGCCCCCC
ACAGAGCGAGGACCCAGAAGAAGTGGGGGAGGGGGAAATGGGAGCCAGAACCCAGTAGCAAGCCCCCC
CTCGGGTGAAATTATCAGATGCGAGAAGTGCAAGAGAGAGAGGGAGCTTCAGCAGAGCCTGGAGCGTG
AGAGGCTTTCTCTGGGGACCAGTGAGCTGGATATGGGGAAGGGCCCAATGTATGATGTGGAGAAGCTG
GTGAGGACCAGAAGCTGCAGGAGGTCTCCCGAGGCAAATCCTGCAAGTGGGGAGGAAGGGTGGAAGGG
TGACAGCCACAGGAGCAGCCCCAGGAATCCCACTCAAGAGCTGAGGAGACCCAGCAAGAGCATGGACA
AGAAAGAGGACAGAGGCCCAGAGGATCAAGAAAGCCATGCTCAGGGAGCAGCCAAGGCCAAGAAGGAC
CTTGTGGAAGTTCTTCCTGTCACAGAGGAGGGGCTGAGGGAGGTGAAGAAGGACACCAGGCCCATGAG
CAGGAGCAAACATGGTGGCTGGCTCCTGAGAGAGCACCAGGCGGGCTTTGAGAAGCTCCGCAGGACCC
GAGGAGAAGAGAAGGAGGCAGAGAAGGAGAAAAAGCCATGTATGTCTGGAGGCAGAAGGATGACTCTC
AGAGATGACCAACCTGCAAAGCTAGAAAAGGAGCCCAAGACGAGGCCAGAAGAGAACAAGCCAGAGCG
GCCCAGCGGTCGGAAGCCACGGCCCATGGGCATCATTGCCGCCAATGTGGAAAAGCATTATGAGACTG
GCCGGGTCATTGGGGATGGGAACTTTGCTGTCGTGAAGGAGTGCAGACACCGCGAGACCAGGCAGGCC
TATGCGATGAAGATCATTGACAAGTCCAGACTCAAGGGCAAGGAGGACATGGTGGACAGTGAGATCTT
GATCATCCAGAGCCTCTCTCACCCCAACATCGTGAAATTGCATGAAGTCTACGAAACAGACATGGAAA
TCTACCTGATCCTGGAGTACGTGCAGGGAGGAGACCTTTTTGACGCCATCATAGAAAGTGTGAAGTTC
CCGGAGCCCGATGCTGCCCTCATGATCATGGACTTATGCAAAGCCCTCGTCCACATGCACGACAAGAG
CATTGTCCACCGGGACCTCAAGCCGGAAAACCTTTTGGTTCAGCGAAATGAGGACAAATCTACTACCT
TGAAATTGGCTGATTTTGGACTTGCAAAGCATGTGGTGAGACCTATATTTACTGTGTGTGGGACCCCA
ACTTACGTAGCTCCCGAAATTCTTTCTGAGAAAGGTTATGGACTGGAGGTGGACATGTGGGCGGCTGG
CGTGATCCTCTATATCCTGCTGTGTGGCTTTCCCCCATTCCGCAGCCCTGAGAGGGACCAGGACGAGC
TCTTTAACATCATCCAGCTGGGCCACTTTGAGTTCCTCCCCCCTTACTGGGACAATATCTCTGATGCT
GCTAAAGATCTGGTGAGCCGGTTGCTGGTGGTAGACCCCAAAAAGCGCTACACAGCTCATCAGGTTCT
TCAGCACCCCTGGATCGAAACAGCTGGCAAGACCAATACAGTGAAACGACAGAAGCAGGTGTCCCCCA
GCAGCGAGGGTCACTTCCGGAGCCAGCACAAGAGGGTTGTGGAGCAGGTATCATAGTCACCACCTTGG
GAATCTGTCCAGCCCCCAGTTCTGCTCAAGGACAGAGAAAAGGATAGAAGTTTGAGAGAAAAACAATG
AAAGAGGCTTCTTCACATAATTGGTGAATCAGAGGGAGAGACACTGAGTATATTTTAAAGCATATTAA
AAAAATTAAGTCAATGTTAAATGTCACAACATATTTTTAGATTTGTATATTTAAAGCCTTTAATACAT
TTTTGGGGGGTAAGCATTGTCATCAGTGAGGAATTTTGGTAATAATGATGTGTTTTGCTTCCCCTTTG
TAACCAAGTTTATTCTGTACTACAGGAGTGGTGCTTACCAGGGTCTAAACTCCCCCTGTGAGATTAAT
AAGGTGCATTGTGGTCTTTCTGTGTTAATAAAATGTGCTCTGAATAACAGAAAAAAAAAAAAAAAAAA
AAAAAAAAAAAAAAAAAAGG

+TL, NOV64a, CG57109-01

| | | | |
|---|---|---|---|
| Protein Sequence | SEQ ID NO: 956 | 648 aa | MW at 73813.6kD |

MGKEPLTLKSIQVAVEELYPNKARALTLAQHSRAPSPRLRSRLFSKALKGDHRCGETETPKSCSEVAG
CKAAMRHQGKIPEELSLDDRARTQKKWGRGKWEPEPSSKPPREATLEERHARGEKHLGVEIEKTSGEI
IRCEKCKRERELQQSLERERLSLGTSELDMGKGPMYDVEKLVRTRSCRRSPEANPASGEEGWKGDSHR
SSPRNPTQELRRPSKSMDKKEDRGPEDQESHAQGAAKAKKDLVEVLPVTEEGLREVKKDTRPMSRSKH
GGWLLREHQAGFEKLRRTRGEEKEAEKEKKPCMSGGRRMTLRDDQPAKLEKEPKTRPEENKPERPSGR
KPRPMGIIAANVEKHYETGRVIGDGNFAVVKECRHRETRQAYAMKIIDKSRLKGKEDMVDSEILIIQS
LSHPNIVKLHEVYETDMEIYLILEYVQGGDLFDAIIESVKFPEPDAALMIMDLCKALVHMHDKSIVHR
DLKPENLLVQRNEDKSTTLKLADFGLAKHVVRPIFTVCGTPTYVAPEILSEKGYGLEVDMWAAGVILY
ILLCGFPPFRSPERDQDELFNIIQLGHFEFLPPYWDNISDAAKDLVSRLLVVDPKKRYTAHQVLQHPW
IETAGKTNTVKRQKQVSPSSEGHFRSQHKRVVEQVS

| | | |
|---|---|---|
| NOV64b, 260457400 | SEQ ID NO: 957 | 793 bp |

| | | |
|---|---|---|
| DNA Sequence | ORF Start: at 2 | ORF Stop: end of sequence |

CACCGGATCCTATGAGACTGGCCGGGTCATTGGGGATGGGAACTTTGCTGTCGTGAAGGAGTGCAGAC
ACCGCGAGACCAGGCAGGCCTATGCGATGAAGATCATTGACAAGTCCAGACTCAAGGGCAAGGAGGAC
ATGGTGGACAGTGAGATCTTGATCATCCAGAGCCTCTCTCACCCCAACATCGTGAAATTGCATGAAGT
CTACGAAACAGACATGGAAATCTACCTGATCCTGGAGTACGTGCAGGGAGGAGACCTTTTTGACGCCA
TCATAGAAAGTGTGAAGTTCCCGGAGCCCGATGCTGCCCTCATGATCATGGACTTATGCAAAGCCCTC
GTCCACATGCACGACAAGAGCATTGTCCACCGGGACCTCAAGCCGGAAAACCTTTTGGTTCAGCGAAA
TGAGGACAAATCTACTACCTTGAAATTGGCTGATTTTGGACTTGCAAAGCATGTGGTGAGACCTATAT
TTACTGTGTGTGGGACCCCAACTTACGTAGCTCCCGAAATTCTTTCTGAGAAAGGTTATGGACTGGAG
GTGGACATGTGGGCTGCTGGCGTGATCCTCTATATCCTGCTGTGTGGCTTTCCCCCATTCCGCAGCCC
TGAGAGGGACCAGGACGAGCTCTTTAACATCATCCAGCTGGGCCACTTTGAGTTCCTCCCCCCTTACT
GGGACAATATCTCTGATGCTGCTAAAGATCTGGTGAGCCGGTTGCTGGTGGTAGACCCCAAAAAGCGC
TACACAGCTCATCAGGTTCTTCAGCACCCCTGGATCCTCGAGGGC

NOV64b, 260457400

| | | | |
|---|---|---|---|
| Protein Sequence | SEQ ID NO: 958 | 264 aa | MW at 30106.4kD |

TGSYETGRVIGDGNFAVVKECRHRETRQAYAMKIIDKSRLKGKEDMVDSEILIIQSLSHPNIVKLHEV
YETDMEIYLILEYVQGGDLFDAIIESVKFPEPDAALMIMDLCKALVHMHDKSIVHRDLKPENLLVQRN
EDKSTTLKLADFGLAKHVVRPIFTVCGTPTYVAPEILSEKGYGLEVDMWAAGVILYILLCGFPPFRSP
ERDQDELFNIIQLGHFEFLPPYWDNISDAAKDLVSRLLVVDPKKRYTAHQVLQHPWILEG

| | | |
|---|---|---|
| NOV64c, 260457409 | SEQ ID NO: 959 | 625 bp |

| | | |
|---|---|---|
| DNA Sequence | ORF Start: at 2 | ORF Stop: end of sequence |

CACCGGATCCTATGAGACTGGCCGGGTCATTGGGGATGGGAACTTTGCTGTCGTGAAGGAGTGCAGAC
ACCGCGAGACCAGGCAGGCCTATGCGATGAAGATCATTGACAAGTCCAGACTCAAGGGCAAGGAGGAC
ATGGTGGACAGTGAGATCTTGATCATCCAGAGCCTCTCTCACCCCAACATCGTGAAATTGCATGAAGT
CTACGAAACAGACATGGAAATCTACCTGATCCTGGAGTACGTGCAGGGAGGAGACCTTTTTGACGCCA
TCATAGAAAGTGTGAAGTTCCCGGAGCCCGATGCTGCCCTCATGATCATGGACTTATGCAAAGCCCTC
GTCCACATGCACGACAAGAGCATTGTCCACCGGGACCTCAAGCCGGAAAACCTTTTGGTTCAGCGAAA
TGAGGACAAATCTACTACCTTGAAATTGGCTGATTTTGGACTTGCAAAGCATGTGGTGAGACCTATAT
TTACTGTGTGTGGGACCCCAACTTACGTAGCTCCCGAAATTCTTTCTGAGAAAGCTGCTAAAGATCTG
GTGAGCCGGTTGCTGGTGGTAGACCCCAAAAAGCGCTACACAGCTCATCAGGTTCTTCAGCACCCCTG
GATCCTCGAGGGC

NOV64c, 260457409

| | | | |
|---|---|---|---|
| Protein Sequence | SEQ ID NO: 960 | 208 aa | MW at 23605.1kD |

TGSYETGRVIGDGNFAVVKECRHRETRQAYAMKIIDKSRLKGKEDMVDSEILIIQSLSHPNIVKLHEV
YETDMEIYLILEYVQGGDLFDAIIESVKFPEPDAALMIMDLCKALVHMHDKSIVHRDLKPENLLVQRN
EDKSTTLKLADFGLAKHVVRPIFTVCGTPTYVAPEILSEKAAKDLVSRLLVVDPKKRYTAHQVLQHPW
ILEG

| | | |
|---|---|---|
| NOV64d, CG57109-05 | SEQ ID NO: 961 | 2133 bp |

| | | |
|---|---|---|
| DNA Sequence | ORF Start: ATG at 90 | ORF Stop: TAG at 2034 |

TTGACCGTGTGAGGAAACTGTTTAACCTCAAGGGCAGGGAAATCAGGAGCGTCTCTGATTTCTTCAGG
GAAGGGGATGCTTTCATAGCTATGGGCAAAGAACCACTGACACTGAAGAGCATTCAGGTGGCTGTAGA
AGAACTGTACCCCAACAAAGCCCGGGCCCTGACACTGGCCCAGCACAGCCGTGCCCCTTCTCCAAGGC
TGAGGAGCAGGCTGTTTAGCAAGGCTCTGAAAGGAGACCACCGCTGTGGGGAGACCGAGACCCCCAAG
AGCTGCAGCGAAGTTGCAGGATGCAAGGCAGCCATGAGGCACCAGGGGAAGATCCCCGAGGAGCTTTC
ACTAGATGACAGAGCGAGGACCCAGAAGAAGTGGGGGAGGGGGAAATGGGAGCCAGAACCCAGTAGCA
AGCCCCCCAGGGAAGCCACTCTGGAAGAGAGGCACGCAAGGGGAGAGAAGCATCTTGGGGTGGAGATT
GAAAAGACCTCGGGTGAAATTATCAGATGCGAGAAGTGCAAGAGAGAGAGGGAGCTCCAGCAGAGCCT
GGAGCGTGAGAGGCTTTCTCTGGGGACCAGTGAGCTGGATATGGGGAAGGGCCCAATGTATGATGTGG
AGAAGCTGGTGAGGACCAGAAGCTGCAGGAGGTCTCCCGAGGCAAATCCTGCAAGTGGGGAGGAAGGG
TGGAAGGGTGACAGCCACAGGAGCAGCCCCAGGAATCCCACTCAAGAGCTGAGGAGACCCAGCAAGAG
CATGGACAAGAAAGAGGACAGAGGCCCAGAGGATCAAGAAAGCCATGCTCAGGGAGCGGCCAAGGCCA
AGAAGGACCTTGTGGAAGTTCTTCCTGTCACAGAGGAGGGGCTGAGAGAGGTGAAGAAGGACACCAGG
CCCATGAGCAGGAGCAAACATGGTGGCTGGCTCCTGAGAGAGCACCAGGCGGGCTTTGAGAAGCTCCG
CAGGACCCGAGGAGAAGAGAAGGAGGCAGAGAAGGAGAAAAAGCCATGTATGTCTGGAGGCAGAAGGA
TGACTCTCAGAGACGACCAACCTGCAAAGCTAGAAAAGGAGCCCAAGACGAGGCCAGAAGAGAACAAG
CCAGAGCGGCCCAGCGGTCGGAAGCCACGGCCCATGGGCATCATTGCCGCCAATGTGGAAAAGCATTA
TGAGACTGGCCGGGTCATTGGGGATGGGAACTTTGCTGTCGTGAAGGAGTGCAGACACCGCGAGACCA
GGCAGGCCTATGCGATGAAGATCATTGACAAGTCCAGACTCAAGGGCAAGGAGGACATGGTGGACAGT
GAGATCTTGATCATCCAGAGCCTCTCTCACCCCAACATCGTGAAATTGCATGAAGTCTACGAAACAGA
CATGGAAATCTACCTGATCCTGGAGTACGTGCAGGGAGGAGACCTTTTTGACGCCATCATAGAAAGTG
TGAAGTTCCCGGAGCCCGATGCTGCCCTCATGATCATGGACTTATGCAAAGCCCTCGTCCACATGCAC
GACAAGAGCATTGTCCACCGGGACCTCAAGCCGGAAAACCTTTTGGTTCAGCGAAATGAGGACAAATC
TACTACCTTGAAATTGGCTGATTTTGGACTTGCAAAGCATGTGGTGAGACCTATATTTACTGTGTGTG
GGACCCCAACTTACGTAGCTCCCGAAATTCTTTCTGAGAAAGGTTATGGACTGGAGGTGGACATGTGG
GCTGCTGGCGTGATCCTCTATATCCTGCTGTGTGGCTTTCCCCCATTCCGCAGCCCTGAGAGGGACCA
GGACGAGCTCTTTAACATCATCCAGCTGGGCCACTTTGAGTTCCTCCCCCCTTACTGGGACAATATCT
CTGATGCTGCTAAAGATCTGGTGAGCCGGTTGCTGGTGGTAGACCCCAAAAAGCGCTACACAGCTCAT
CAGGTTCTTCAGCACCCCTGGATCGAAACAGCTGGCAAGACCAATACAGTGAAACGACAGAAGCAGGT
GTCCCCCAGCAGCGAGGGTCACTTCCGGAGCCAGCACAAGAGGGTTGTGGAGCAGGTATCATAGTCAC
CACCTTGGGAATCTGTCCAGCCCCCAGTTCTGCTCAAGGACAGAGAAAAGGATAGAAGTTTGAGAGAA
AAACAATGAAAGAGGCTTCTTCACA
NOV64d, CG57109-05

| | | | |
|---|---|---|---|
| Protein Sequence | SEQ ID NO: 962 | 648 aa | MW at 73813.6kD |

MGKEPLTLKSIQVAVEELYPNKARALTLAQHSRAPSPRLRSRLFSKALKGDHRCGETETPKSCSEVAG
CKAAMRHQGKIPEELSLDDRARTQKKWGRGKWEPEPSSKPPREATLEERHARGEKHLGVEIEKISGEI
IRCEKCKRERELQQSLERERLSLGTSELDMGKGPMYDVEKLVRTRSCRRSPEANPASGEEGWKGDSHR
SSPRNPTQELRRPSKSMDKKEDRGPEDQESHAQGAAKAKKDLVEVLPVTEEGLREVKKDTRPMSRSKH
GGWLLREHQAGFEKLRRTRGEEKEAEKEKKPCMSGGRRMTLRDDQPAKLEKEPKTRPEENKPERPSGR
KPRPMGIIAANVEKHYETGRVIGDGNFAVVKECRHRETRQAYAMKIIDKSRLKGKEDMVDSEILIIQS
LSHPNIVKLHEVYETDMEIYLILEYVQGGDLFDAIIESVKFPEPDAALMIMDLCKALVHMHDKSIVHR
DLKPENLLVQRNEDKSTTLKLADFGLAKHVVRPIFTVCGTPTYVAPEILSEKGYGLEVDMWAAGVILY
ILLCGFPPFRSPERDQDELFMIIQLGHFEFLPPYWDNISDAAKDLVSRLLVVDPKKRYTAHQVLQHPW
IETAGKTNTVKRQKQVSPSSEGHFRSQHKRVVEQVS

| | | |
|---|---|---|
| NOV64e, 267253965 | SEQ ID NO: 963 | 1966 bp |

| | | |
|---|---|---|
| DNA Sequence | ORF Start: at 2 | ORF Stop: end of sequence |

CACCGGATCCACCATGGGCAAAGAACCACTGACACTGAAGAGCATTCAGGTGGCTGTAGAAGAACTGT
ACCCCAACAAAGCCCGGGCCCTGACACTGGCCCAGCACAGCCGTGCCCCTTCTCCAAGGCTGAGGAGC
AGGCTGTTTAGCAAGGCTCTGAAAGGAGACCACCGCTGTGGGGAGACCGAGACCCCCAAGAGCTGCAG
CGAAGTTGCAGGATGCAAGGCAGCCATGAGGCACCAGGGGAAGATCCCCGAGGAGCTTTCACTAGATG
ACAGAGCGAGGACCCAGAAGAAGTGGGGGAGGGGGAAATGGGAGCCAGAACCCAGTAGCAAGCCCCCC
AGGGAAGCCACTCTGGAAGAGAGGCACGCAAGGGGAGAGAAGCATCTTGGGGTGGAGATTGAAAAGAC
CTCGGGTGAAATTATCAGATGCGAGAAGTGCAAGAGAGAGAGGGAGCTCCAGCAGAGCCTGGAGCGTG
AGAGGCTTTCTCTGGGGACCAGTGAGCTGGATATGGGGAAGGGCCCAATGTATGATGTGGAGAAGCTG
GTGAGGACCAGAAGCTGCAGGAGGTCTCCCGAGGCAAATCCTGCAAGTGGGGAGGAAGGGTGGAAGGG
TGACAGCCACAGGAGCAGCCCCAGGAATCCCACTCAAGAGCTGAGGAGACCCAGCAAGAGCATGGACA
AGAAAGAGGACAGAGGCCCAGAGGATCAAGAAAGCCATGCTCAGGGAGCGGCCAAGGCCAAGAAGGAC
CTTGTGGAAGTTCTTCCTGTCACAGAGGAGGGGCTGAGAGAGGTGAAGAAGGACACCAGGCCCATGAG
CAGGAGCAAACATGGTGGCTGGCTCCTGAGAGAGCACCAGGCGGGCTTTGAGAAGCTCCGCAGGACCC
GAGGAGAAGAGAAGGAGGCAGAGAAGGAGAAAAAGCCATGTATGTCTGGAGGCAGAAGGATGACTCTC
AGAGACGACCAACCTGCAAAGCTAGAAAAGGAGCCCAAGACGAGGCCAGAAGAGAACAAGCCAGAGCG
GCCCAGCGGTCGGAAGCCACGGCCCATGGGCATCATTGCCGCCAATGTGGAAAAGCATTATGAGACTG
GCCGGGTCATTGGGGATGGGAACTTTGCTGTCGTGAAGGAGTGCAGACACCGCGAGACCAGGCAGGCC
TATGCGATGAAGATCATTGACAAGTCCAGACTCAAGGGCAAGGAGGACATGGTGGACAGTGAGATCTT
GATCATCCAGAGCCTCTCTCACCCCAACATCGTGAAATTGCATGAAGTCTACGAAACAGACATGGAAA
TCTACCTGATCCTGGAGTACGTGCAGGGAGGAGACCTTTTTGACGCCATCATAGAAAGTGTGAAGTTC
CCGGAGCCCGATGCTGCCCTCATGATCATGGACTTATGCAAAGCCCTCGTCCACATGCACGACAAGAG
CATTGTCCACCGGGACCTCAAGCCGGAAAACCTTTTGGTTCAGCGAAATGAGGACAAATCTACTACCT
TGAAATTGGCTGATTTTGGACTTGCAAAGCATGTGGTGAGACCTATATTTACTGTGTGTGGGACCCCA
ACTTACGTAGCTCCCGAAATTCTTTCTGAGAAAGGTTATGGACTGGAGGTGGACATGTGGGCTGCTGG
CGTGATCCTCTATATCCTGCTGTGTGGCTTTCCCCCATTCCGCAGCCCTGAGAGGGACCAGGACGAGC
TCTTTAACATCATCCAGCTGGGCCACTTTGAGTTCCTCCCCCCTTACTGGGACAATATCTCTGATGCT
GCTAAAGATCTGGTGAGCCGGTTGCTGGTGGTAGACCCCAAAAAGCGCTACACAGCTCATCAGGTTCT
TCAGCACCCCTGGATCGAAACAGCTGGCAAGACCAATACAGTGAAACGACAGAAGCAGGTGTCCCCCA
GCAGCGAGGGTCACTTCCGGAGCCAGCACAAGAGGGTTGTGGAGCAGGTATCACTCGAGGGC

NOV64e, 267253965

| | | | |
|---|---|---|---|
| Protein Sequence | SEQ ID NO: 964 | 655 aa | MW at 74459.2kD |

TGSTMGKEPLTLKSIQVAVEELYPNKARALTLAQHSRAPSPRLRSRLFSKALKGDHRCGETETPKSCS
EVAGCKAAMRHQGKIPEELSLDDRARTQKKWGRGKWEPEPSSKPPREATLEERHARGEKHLGVEIEKT
SGEIIRCEKCKRERELQQSLERERLSLGTSELDMGKGPMYDVEKLVRTRSCRRSPEANPASGEEGWKG
DSHRSSPRNPTQELRRPSKSMDKKEDRGPEDQESHAQGAAKAKKDLVEVLPVTEEGLREVKKDTRPMS
RSKHGGWLLREHQAGFEKLRRTRGEEKEAEKEKKPCMSGGRRMTLRDDQPAKLEKEPKTRPEENKPER
PSGRKPRPMGIIAANVEKHYETGRVIGDGNFAVVKECRHRETRQAYANKIIDKSRLKGKEDMVDSEIL
IIQSLSHPNIVKLHEVYETDMEIYLILEYVQGGDLFDAIIESVKFPEPDAALMIMDLCKALVHMHDKS
IVHRDLKPENLLVQRNEDKSTTLKLADFGLAKHVVRPIFTVCGTPTYVAPEILSEKGYGLEVDMWAAG
VILYILLCGFPPFRSPERDQDELFNIIQLGHFEFLPPYWDNISDAAKDLVSRLLVVDPKKRYTAHQVL
QHPWIETAGKTNTVKRQKQVSPSSEGHFRSQHKRVVEQVSLEG

| | | |
|---|---|---|
| NOV64f, 267254000 | SEQ ID NO: 965 | 1621 bp |

| | | |
|---|---|---|
| DNA Sequence | ORF Start: at 2 | ORF Stop: end of sequence |

CACCGGATCCACCATGGGCAAAGAACCACTGACACTGAAGAGCATTCAGGTGGCTGTAGAAGAACTGT
ACCCCAACAAAGCCCGGGCCCTGACACTGGCCCAGCACAGCCGTGCCCCTTCTCCAAGGCTGAGGAGC
AGGCTGTTTAGCAAGGCTCTGAAAGGAGACCACCGCTGTGGGGAGACCGAGACCCCCAAGAGCTGCAG
CGAAGTTGCAGGATGCAAGGCAGCCATGAGGCACCAGGGGAAGATCCCCGAGGAGCTTTCACTAGATG
ACAGAGCGAGGACCCAGAAGAAGTGGGGGAGGGGGAAATGGGAGCCAGAACCCAGTAGCAAGCCCCCC
AGGGAAGCCACTCTGGAAGAGAGGCACGCAAGGGGAGAGAAGCATCTTGGGGTGGAGATTGAAAAGAC
CTCGGGTGAAATTATCAGATGCGAGAAGTGCAAGAGAGAGAGGGAGCTCCAGCAGAGCCTGGAGCGTG
AGAGGCTTTCTCTGGGGACCAGTGAGCTGGATATGGGGAAGGGCCCAATGTATGATGTGGAGAAGCTG
GTGAGGACCAGAAGCTGCAGGAGGTCTCCCGAGGCAAATCCTGCAAGTGGGGAGGAAGGGTGGAAGGG
TGACAGCCACAGGAGCAGCCCCAGGAATCCCACTCAAGAGCTGAGGAGACCCAGCAAGAGCATGGACA
AGAAAGAGGACAGAGGCCCAGAGGATCAAGAAAGCCATGCTCAGGGAGCGGCCAAGGCCAAGAAGGAC
CTTGTGGAAGTTCTTCCTGTCACAGAGGAGGGGCTGAGAGAGGTGAAGAAGGACACCAGGCCCATGAG
CAGGAGCAAACATGGTGGCTGGCTCCTGAGAGAGCACCAGGCGGGCTTTGAGAAGCTCCGCAGGACCC
GAGGAGAAGAGAAGGAGGCAGAGAAGGAGAAAAAGCCATGTATGTCTGGAGGCAGAAGGATGACTCTC
AGAGACGACCAACCTGCAAAGCTAGAAAAGGAGCCCAAGACGAGGCCAGAAGAGAACAAGCCAGAGCG
GCCCAGCGGTCGGAAGCCACGGCCCATGGGCATCATTGCCGCCAATGTGGAAAAGCATTATGAGACTG
GCCGGGTCATTGGGGATGGGAACTTTGCTGTCGTGAAGGAGTGCAGACACCGCGAGACCAGGCAGGCC
TATGCGATGAAGATCATTGACAAGTCCAGACTCAAGGGCAAGGAGGACATGGTGGACAGTGAGATCTT
GATCATCCAGAGCCTCTCTCACCCCAACATCGTGAAATTGCATGAAGTCTACGAAACAGACATGGAAA
TCTACCTGATCCTGGAGTACGTGCAGGGAGGAGACCTTTTTGACGCCATCATAGAAAGTGTGAAGTTC
CCGGAGCCCGATGCTGCCCTCATGATCATGGACTTATGCAAAGCCCTCGTCCACATGCACGACAAGAG
CATTGTCCACCGGGACCTCAAGCCGGAAAACCTTTTGGTTCAGCGAAATGAGGACAAATCTACTACCT
TGAAATTGGCTGATTTTGGACTTGCAAAGCATGTGGTGAGACCTATATTTACTGTGTGTGGGACCCCA
ACTTACGTAGCTCCCGAAATTCTTTCTGAGAAAGGTTATGGACTGGAGCTCGAGGGC

NOV64f, 267254000

| | | | |
|---|---|---|---|
| Protein Sequence | SEQ ID NO: 966 | 540 aa | MW at 61179.2kD |

TGSTMGKEPLTLKSIQVAVEELYPNKARALTLAQHSRAPSPRLRSRLFSKALKGDHRCGETETPKSCS
EVAGCKAAMRHQGKIPEELSLDDRARTQKKWGRGKWEPEPSSKPPREATLEERHARGEKHLGVEIEKT
SGEIIRCEKCKRERELQQSLERERLSLGTSELDMGKGPMYDVEKLVRTRSCRRSPEANPASGEEGWKC
DSHRSSPRNPTQELRRPSKSMDKKEDRGPEDQESHAQGAAKAKKDLVEVLPVTEEGLREVKKDTRPMS
RSKHGGWLLREHQAGFEKLRRTRGEEKEAEKEKKPCMSGGRRMTLRDDQPAKLEKEPKTRPEENKPER
PSGRKPRPMGIIAANVEKHYETGRVIGDGNFAVVKECRHRETRQAYANKIIDKSRLKGKEDMVDSEIL
IIQSLSHPNIVKLHEVYETDMEIYLILEYVQGGDLFDAIIESVKFPEPDAALMIMDLCKALVHMHDKS
IVHRDLKPENLLVQRNEDKSTTLKLADFGLAKHVVRPIFTVCGTPTYVAPEILSEKGYGLELEG

| | | |
|---|---|---|
| NOV64g, 267253987 | SEQ ID NO: 967 | 793 bp |

| | | |
|---|---|---|
| DNA Sequence | ORF Start: at 1 | ORF Stop: at 793 |

ACCGGATCCTATGAGACTGGCCGGGTCATTGGGGATGGGAACTTTGCTGTCGTGAAGGAGTGCAGACA
CCGCGAGACCAGGCAGGCCTATGCGATGAAGATCATTGACAAGTCCAGACTCAAGGGCAAGGAGGACA
TGGTGGACAGTGAGATCTTGATCATCCAGAGCCTCTCTCACCCCAACATCGTGAAATTGCATGAAGTC
TACGAAACAGACATGGAAATCTACCTGATCCTGGAGTACGTGCAGGGAGGAGACCTTTTTGACGCCAT
CATAGAAAGTGTGAAGTTCCCGGAGCCCGATGCTGCCCTCATGATCATGGACTTATGCAAAGCCCTCG
TCCACATGCACGACAAGAGCATTGTCCACCGGGACCTCAAGCCGGAAAACCTTTTGGTTCAGCGAAAT
GAGGACAAATCTACTACCTTGAAATTGGCTGATTTTGGACTTGCAAAGCATGTGGTGAGACCTATATT
TACTGTGTGTGGGACCCCAACTTACGTAGCTCCCGAAATTCTTTCTGAGAAAGGTTATGGACTGGAGG
TGGACATGTGGGCTGCTGGCGTGATCCTCTATATCCTGCTGTGTGGCTTTCCCCCATTCCGCAGCCCT
GAGAGGGACCAGGACGAGCTCTTTAACATCATCCAGCTGGGCCACTTTGAGTTCCTCCCCCCTTACTG
GGACAATATCTCTGATGCTGCTAAAGATCTGGTGAGCCGGTTGCTGGTGGTAGACCCCAAAAAGCGCT
ACACAGCTCATCAGGTTCTTCAGCACCCCTGGATCCTCGAGGGCC

NOV64g, 267253987

| | | | |
|---|---|---|---|
| Protein Sequence | SEQ ID NO: 968 | 264 aa | MW at 30106.4kD |

TGSYETGRVIGDGNFAVVKECRHRETRQAYANKIIDKSRLKGKEDMVDSEILIIQSLSHPNIVKLHEV
YETDMEIYLILEYVQGGDLFDAIIESVKFPEPDAALMIMDLCKALVHMHDKSIVHRDLKPENLLVQRN
EDKSTTLKLADFGLAKHVVRPIFTVCGTPTYVAPEILSEKGYGLEVDMWAAGVILYILLCGFPPFRSP
ERDQDELFNIIQLGHFEFLPPYWDNISDAAKDLVSRLLVVDPKKRYTAHQVLQHPWILEG

| | | |
|---|---|---|
| NOV64h, CG57109-02 | SEQ ID NO: 969 | 2808 bp |

| | | |
|---|---|---|
| DNA Sequence | ORF Start: ATG at 151 | ORF Stop: TAG at 2659 |

TTTACAGAGTCAGGCCTCACCGTGAGAGGGCTCCTGTATTAGTCCCTTTTCATGCTGCTTATAGAGAC
ATACCTGAGACTGGGCAATTTGCAGAGAAAGGTTTTCTTGGACTTACAGTTAGTTCCACGTGGCTGGG
GAAGCCTCACAATCATGGCGGAAGGCAAGGAAGGGCAAGTCCCATCTTACATGGATGGCAGCAGGCAA
AGAGAGAATGAGGAAGATGCAAAAGCGGAAACCCCTGATGTAACCATCAGATCTTATGAGATTTATTC
ACTACCATGGAACAGACAGCAAGGCGTATGTGACCATTCTCTAGAATATTTAAGCTCGAGAATCTCAG
AGCGGAAGCTGCAAGGCTCCTGGCTGCCTGCCAGCCGAGGGAATCTGGAGAAACCATTCCTGGGGCCG
CGTGGCCCCGTCGTGCCCTTGTTCTGCCCTCGGAATGGCCTTCACTCAGCACATCCTGAGAACAGCCC
TCTGAAGCCCAGGGTCGTGACCGTAGTGAAGCTGGGTGGGCAGCGCCCCCGAAAGATCACTCTGCTCC
TCAACAGGCGATCAGTGCAGACGTTCGAGCAGCTCTTAGCTGACATCTCAGAAGCCTTGGGCTCTCCC
AGATGGAAGAATGACCGTGTGAGGAAACTGTTTAACCTCAAGGGCAGGGAAATCAGGAGCGTCTCTGA
TTTCTTCAGGGAAGGGGATGCTTTCATAGCTATGGGCAAAGAACCACTGACACTGAAGAGCATTCAGG
TGGCTGTAGAAGAACTGTACCCCAACAAAGCCCGGGCCCTGACACTGGCCCAGCACAGCCGTGCCCCT
TCTCCAAGGCTGAGGAGCAGGCTGTTTAGCAAGGCTCTGAAAGGAGACCACCGCTGTGGGGAGACCGA
GACCCCCAAGAGCTGCAGCGAAGTTGCAGGATGCAAGGCAGCCATGAGGCACCAGGGGAAGATCCCCG
AGGAGCTTTCACTAGATGACAGAGCGAGGACCCAGAAGAAGTGGGGGAGGGGGAAATGGGAGCCAGAA
CCCAGTAGCAAGCCCCCCAGGGAAGCCACTCTGGAAGAGAGGCACGCAAGGGGAGAGAAGCATCTTGG
GGTGGAGATTGAAAAGACCTCGGGTGAAATTATCAGATGCGAGAAGTGCAAGAGAGAGAGGGAGCTCC
AGCAGAGCCTGGAGCGTGAGAGGCTTTCTCTGGGGACCAGTGAGCTGGATATGGGGAAGGGCCCAATG
TATGATGTGGAGAAGCTGGTGAGGACCAGAAGCTGCAGGAGGTCTCCCGAGGCAAATCCTGCAAGTGG
GGAGGAAGGGTGGAAGGGTGACAGCCACAGGAGCAGCCCCAGGAATCCCACTCAAGAGCTGAGGAGAC
CCAGCAAGAGCATGGACAAGAAAGAGGACAGAGGCCCAGAGGATCAAGAAAGCCATGCTCAGGGAGCA
GCCAAGGCCAAGAAGGACCTTGTGGAAGTTCTTCCTGTCACAGAGGAGGGGCTGAGGGAGGTGAAGAA
GGACACCAGGCCCATGAGCAGGAGCAAACATGGTGGCTGGCTCCTGAGAGAGCACCAGGCGGGCTTTG
AGAAGCTCCGCAGGACCCGAGGAGAAGAGAAGGAGGCAGAGAAGGAGAAAAAGCCATGTATGTCTGGA
GGCAGAAGGATGACTCTCAGAGATGACCAACCTGCAAAGCTAGAAAAGGAGCCCAAGACGAGGCCAGA
AGAGAACAAGCCAGAGCGGCCCAGCGGTCGGAAGCCACGGCCCATGGGCATCATTGCCGCCAATGTGG
AAAAGCATTATGAGACTGGCCGGGTCATTGGGGATGGGAACTTTGCTGTCGTGAAGGAGTGCAGACAC
CGCGAGACCAGGCAGGCCTATGCGATGAAGATCATTGACAAGTCCAGACTCAAGGGCAAGGAGGACAT
GGTGGACAGTGAGATCTTGATCATCCAGAGCCTCTCTCACCCCAACATCGTGAAATTGCATGAAGTCT
ACGAAACAGACATGGAAATCTACCTGATCCTGGAGTACGTGCAGGGAGGAGACCTTTTTGACGCCATC
ATAGAAAGTGTGAAGTTCCCGGAGCCCGATGCTGCCCTCATGATCATGGACTTATGCAAAGCCCTCGT
CCACATGCACGACAAGAGCATTGTCCACCGGGACCTCAAGCCGGAAAACCTTTTGGTTCAGCGAAATG
AGGACAAATCTACTACCTTGAAATTGGCTGATTTTGGACTTGCAAAGCATGTGGTGAGACCTATATTT
ACTGTGTGTGGGACCCCAACTTACGTAGCTCCCGAAATTCTTTCTGAGAAAGGTTATGGACTGGAGGT
GGACATGTGGGCTGCTGGCGTGATCCTCTATATCCTGCTGTGTGGCTTTCCCCCATTCCGCAGCCCTG
AGAGGGACCAGGACGAGCTCTTTAACATCATCCAGCTGGGCCACTTTGAGTTCCTCCCCCCTTACTGG
GACAATATCTCTGATACAGCTGCTAAAGATCTGGTGAGCCGGTTGCTGGTGGTAGACCCCAAAAAGCG
CTACACAGCTCATCAGGTTCTTCAGCACCCCTGGATCGAAACAGCTGGCAAGACCAATACAGTGAAAC
GACAGAAGCAGGTGTCCCCCAGCAGCGAGGGTCACTTCCGGAGCCAGCACAAGAGGGTTGTGGAGCAG
GTATCATAGTCACCACCTTGGGAATCTGTCCAGCCCCCAGTTCTGCTCAAGGACAGAGAAAAGGATAG
AAGTTTGAGAGAAAAACAATGAAAGAGGCTTCTTCACATAATTGGTGAATCAGAGGGAGAGACACTGA
GTATATTTTAAAGCATATTA

NOV64h, CG57109-02

| | | | |
|---|---|---|---|
| Protein Sequence | SEQ ID NO: 970 | 836 aa | MW at 95152.6kD |

MAEGKEGQVPSYMDGSRQRENEEDAKAETPDVTIRSYEIYSLPWNRQQGVCDHSLEYLSSRISERKLQ
GSWLPASRGNLEKPFLGPRGPVVPLFCPRNGLHSAHPENSPLKPRVVTVVKLGGQRPRKITLLLNRRS
VQTFEQLLADISEALGSPRWKNDRVRKLFNLKGREIRSVSDFFREGDAFIAMGKEPLTLKSIQVAVEE
LYPNKARALTLAQHSRAPSPRLRSRLFSKALKGDHRCGETETPKSCSEVAGCKAAMRHQGKIPEELSL
DDRARTQKKWGRGKWEPEPSSKPPREATLEERHARGEKHLGVEIEKTSGEIIRCEKCKRERELQQSLE
RERLSLGTSELDMGKGPMYDVEKLVRTRSCRRSPEANPASGEEGWKGDSHRSSPRNPTQELRRPSKSM
DKKEDRGPEDQESHAQGAAKAKKDLVEVLPVTEEGLREVKKDTRPMSRSKHGGWLLREHQAGFEKLRR
TRGEEKEAEKEKKPCMSGGRRNTLRDDQPAKLEKEPKTRPEENKPERPSGRKPRPMGIIAANVEKHYE
THRVIGDGNFAVVKECRHRETRQAYAMKIIDKSRLKGKEDMVDSEILIIQSLSHPNIVKLHEVYETDM
EIYLILEYVQGGDLFDAIIESVKFPEPDAALMIMDLCKALVHMDDKSIVHRDLKPENLLVQRNEDKST
TLKLADFGLAKHVVRPIFTVCGTPTYVAPEILSEKGYGLEVDMWAAGVILYILLCGFPPFRSPERDQD
ELFNIIQLGHFEFLPPYWDNISDTAAKDLVSRLLVVDPKKRYTAHQVLQHPWIETAGKTNTVKRQKQV
SPSSEGHFRSQHKRVVEQVS

| | | |
|---|---|---|
| NOV64i, CG57109-03 | SEQ ID NO: 971 | 3016 bp |

| | | |
|---|---|---|
| DNA Sequence | ORF Start: ATG at 52 | ORF Stop: TAG at 2617 |

TTTACAGAGTCAGGCCTCACCGTGAGAGGGCTCCTGTATTAGTCCCTTTTCATGCTGCTTATAGAGAC
ATACCTGAGACTGGGCAATTTGCAGAGAAAGGTTTTCTTGGACTTACAGTTAGTTCCACGTGGCTGGG
GAAGCCTCACAATCATGGCGGAAGGCAAGGAAGGGCAAGTCCCATCTTACATGGATGGCAGCAGGCAA
AGAGAGAATGAGGAAGATGCAAAAGCGGAAACCCCTGATGTAACCATCAGATCTTATGAGATTTATTC
ACTACCATGGAACAGACAGCAAGGCGTATGTGACCATTCTCTAGAATATTTAAGCTCGAGAATCTCAG
AGCGGAAGCTGCAAGGCTCCTGGCTGCCTGCCAGCCGAGGGAATCTGGAGAAACCATTCCTGGGGCCG
CGTGGCCCCGTCGTGCCCTTGTTCTGCCCTCGGAATGGCCTTCACTCAGCACATCCTGAGAACAGCCC
TCTGAAGCCCAGGGTCGTGACCGTAGTGAAGCTGGGTGGGCAGCGCCCCCGAAAGATCACTCTGCTCC
TCAACAGGCGATCAGTGCAGACGTTCGAGCAGCTCTTAGCTGACATCTCAGAAGCCTTGGGCTCTCCC
AGATGGAAGAATGACCGTGTGAGGAAACTGTTTAACCTCAAGGGCAGGGAAATCAGGAGCGTCTCTGA
TTTCTTCAGGGAAGGGGATGCTTTCATAGCTATGGGCAAAGAACCACTGACACTGAAGAGCATTCAGG
TGGCTGTAGAAGAACTGTACCCCAACAAAGCCCGGGCCCTGACACTGGCCCAGCACAGCCGTGCCCCT
TCTCCAAGGCTGAGGAGCAGGCTGTTTAGCAAGGCTCTGAAAGGAGACCACCGCTGTGGGGAGACCGA
GACCCCCAAGAGCTGCAGCGAAGTTGCAGGATGCAAGGCAGCTATGAGGCACCAGGGGAAGATCCCCG
AGGAGCTTTCACTAGATGACAGAGCGAGGACCCAGAAGAAGTGGGGGAGGGGGAAATGGGAGCCAGAA
CCCAGTAGCAAGCCCCCCAGGGAAGCCACTCTGGAAGAGAGGCACGCAAGGGGAGAGAAGCATCTTGG
GGTGGAGATTGAAAAGACCTCGGGTGAAATTATCAGATGCGAGAAGTGCAAGAGAGAGAGGGAGCTTC
AGCAGAGCCTGGAGCGTGAGAGGCTTTCTCTGGGGACCAGTGAGCTGGATATGGGGAAGGGCCCAATG
TATGATGTGGAGAAGCTGGTGAGGACCAGAAGCTGCAGGAGGTCTCCCGAGGCAAATCCTGCAAGTGG
GGAGGAAGGGTGGAAGGGTGACAGCCACAGGAGCAGCCCCAGGAATCCCACTCAAGAGCTGAGGAGAC
CCAGCAAGAGCATGGACAAGAAAGAGGACAGAGGCCCAGAGGATCAAGAAAGCCATGCTCAGGGAGCA
GCCAAGGCCAAGAAGGACCTTGTGGAAGTTCTTCCTGTCACAGAGGAGGGGCTGAGGGAGGTGAAGAA
GGACACCAGGCCCATGAGCAGGAGCAAACATGGTGGCTGGCTCCTGAGAGAGCACCAGGCGGGCTTTG
AGAAGCTCCGCAGGACCCGAGGAGAAGAGAAGGAGGCAGAGAAGGAGAAAAAGCCATGTATGTCTGGA
GGCAGAAGGATGACTCTCAGAGATGACCAACCTGCAAAGCTAGAAAAGGAGCCCAAGACGAGGCCAGA
AGAGAACAAGCCAGAGCGGCCCAGCGGTCGGAAGCCACGGCCCATGGGCATCATTGCCGCCAATGTGG
AAAAGCATTATGAGACTGGCCGGGTCATTGGGGATGGGAACTTTGCTGTCGTGAAGGAGTGCAGACAC
CGCGAGACCAGGCAGGCCTATGCGATGAAGATCATTGACAAGTCCAGACTCAAGGGCAAGGAGGACAT
GGTGGACAGTGAGATCTTGCATGAAGTCTACGAAACAGACATGGAAATCTACCTGATCCTGGAGTACG
TGCAGGGAGGAGACCTTTTTGACGCCATCATAGAAAGTGTGAAGTTCCCGGAGCCCGATGCTGCCCTC
ATGATCATGGACTTATGCAAAGCCCTCGTCCACATGCACGACAAGAGCATTGTCCACCGGGACCTCAA
GCCGGAAAACCTTTTGGTTCAGCGAAATGAGGACAAATCTACTACCTTGAAATTGGCTGATTTTGGAC
TTGCAAAGCATGTGGTGAGACCTATATTTACTGTGTGTGGGACCCCAACTTACGTAGCTCCCGAAATT
CTTTCTGAGAAAGGTTATGGACTGGAGGTGGACATGTGGGCTGCTGGCGTGATCCTCTATATCCTGCT
GTGTGGCTTTCCCCCATTCCGCAGCCCTGAGAGGGACCAGGACGAGCTCTTTAACATCATCCAGCTGG
GCCACTTTGAGTTCCTCCCCCCTTACTGGGACAATATCTCTGATGCTGCTAAAGATCTGGTGAGCCGG
TTGCTGGTGGTAGACCCCAAAAAGCGCTACACAGCTCATCAGGTTCTTCAGCACCCCTGGATCGAAAC
AGCTGGCAAGACCAATACAGTGAAACGACAGAAGCAGGTGTCCCCCAGCAGCGAGGGTCACTTCCGGA
GCCAGCACAAGAGGGTTGTGGAGCAGGTATCATAGTCACCACCTTGGGAATCTGTCCAGCCCCCAGTT
CTGCTCAAGGACAGAGAAAAGGATAGAAGTTTGAGAGAAAAACAATGAAAGAGGCTTCTTCACATAAT
TGGTGAATCAGAGGGAGAGACACTGAGTATATTTTAAAGCATATTAAAAAAATTAAGTCAATGTTAAA
TGTCACAACATATTTTTAGATTTGTATATTTAAAGCCTTTAATACATTTTTGGGGGGTAAGCATTGTC
ATCAGTGAGGAATTTTGGTAATAATGATGTGTTTTGCTTCCCCTTTGTAACCAAGTTTATTCTGTACT
ACAGGAGTGGTGCTTACCAGGGTCTAAACTCCCCCTGTGAGATTAATAAGGTGCACTGTGGTCTTTCT
GTGTTAATAAAATGTGCTCTGAAT

NOV64i, CG57109-03

| | | | |
|---|---|---|---|
| Protein Sequence | SEQ ID NO: 972 | 855 aa | MW at 97447.3kD |

MLLIETYLRLGNLQRKVFLDLQLVPRGWGSLTIMAEGKEGQVPSYMDGSRQRENEEDAKAETPDVTIR
SYEIYSLPWNRQQGVCDHSLEYLSSRISERKLQGSWLPASRGNLEKPFLGPRGPVVPLFCPRNGLHSA
HPENSPLKPRVVTVVKLGGQRPRKITLLLNRRSVQTFEQLLADISEALGSPRWKNDRVRKLFNLKGRE
IRSVSDFFREGDAFIAMGKEPLTLKSIQVAVEELYPNKARALTLAQHSRAPSPRLRSRLFSKALKGDH
RCGETETPKSCSEVAGCKAAMRHQGKIPEELSLDDRARTQKKWGRGKWEPEPSSKPPREATLEERHAR
GEKHLGVEIEKTSGEIIRCEKCKRERELQQSLERERLSLGTSELDMGKGPMYDVEKLVRTRSCRRSPE
ANPASGEEGWKGDSHRSSPRNPTQELRRPSKSMDKKEDRGPEDQESHAQGAAKAKKDLVEVLPVTEEG
LREVKKDTRPMSRSKHGGWLLREHQAGFEKLRRTRGEEKEAEKEKKPCMSGGRRNTLRDDQPAKLEKE
PKTRPEENKPERPSGRKPRPMGIIAANVEKHYETGRVIGDGNFAVVKECRHRETRQAYAMKIIDKSRL
KGKEDMVDSEILHEVYETDMEIYLILEYVQGGDLFDAIIESVKFPEPDAALMIMDLCKALVHMHDKSI
VHRDLKPENLLVQRNEDKSTTLKLADFGLAKHVVRPIFTVCGTPTYVAPEILSEKGYGLEVDMWAAGV
ILYILLCGFPPFRSPERDQDELFNIIQLGHFEFLPPYWDNISDAAKDLVSRLLVVDPKKRYTAHQVLQ
HPWIETAGKTNTVKRQKQVSPSSEGHFRSQHKRVVEQVS

| | | |
|---|---|---|
| NOV64j, CG57109-04 | SEQ ID NO: 973 | 2433 bp |

| | | |
|---|---|---|
| DNA Sequence | ORF Start: ATG at 52 | ORF Stop: TAG at 2284 |

TTTACAGAGTCAGGCCTCACCGTGAGAGGGCTCCTGTATTAGTCCCTTTTCATGCTGCTTATAGAGAC
ATACCTGAGACTGGGCAATTTGCAGAGAAAGGTTTTCTTGGACTTACAGTTAGTTCCACGTGGCTGGG
GAAGCCTCACAATCATGGCGGAAGGCAAGGAAGGGCAAGTCCCATCTTACATGGATGGCAGCAGGCAA
AGAGAGAATGAGGAAGATGCAAAAGCGGAAACCCCTGATGTAACCATCAGATCTTATGAGATTTATTC
ACTACCATGGAACAGACAGCAAGGCGTATGTGACCATTCTCTAGAATATTTAAGCTCGAGAATCTCAG
AGCGGAAGCTGCAAGGCTCCTGGCTGCCTGCCAGCCGAGGGAATCTGGAGAAACCATTCCTGGGGCCG
CGTGGCCCCGTCGTGCCCTTGTTCTGCCCTCGGAATGGCCTTCACTCAGCACATCCTGAGAACAGCCC
TCTGAAGCCCAGGGTCGTGACCGTAGTGAAGCTGGGTGGGCAGCGCCCCCGAAAGATCACTCTGCTCC
TCAACAGGCGATCAGTGCAGACGTTCGAGCAGCTCTTAGCTGACATCTCAGAAGCCTTGGGCTCTCCC
AGATGGAAGAATGACCGTGTGAGGAAACTGTTTAACCTCAAGGGCAGGGAAATCAGGAGCGTCTCTGA
TTTCTTCAGGGAAGGGGATGCTTTCATAGCTATGGGCAAAGAACCACTGACACTGAAGAGCATTCAGG
TGGCTGTAGAAGAACTGTACCCCAACAAAGCCCGGGCCCTGACACTGGCCCAGCACAGCCGTGCCCCT
TCTCCAAGGCTGAGGAGCAGGCTGTTTAGCAAGGCTCTGAAAGGAGACCACCGCTGTGGGGAGACCGA
GACCCCCAAGAGCTGCAGCGAAGTTGCAGGATGCAAGGCAGCCATGAGGCACCAGGGGAAGATCCCCG
AGGAGCTTTCACTAGATGACAGAGCGAGGACCCAGAAGAAGTGGGGGAGGGGGAAATGGGAGCCAGAA
CCCAGTAGCAAGCCCCCCAGGGAAGCCACTCTGGAAGAGAGGCACGCAAGGGGAGAGAAGCATCTTGG
GGTGGAGATTGAAAAGACCTCGGGTGAAATTATCAGATGCGAGAAGTGCAAGAGAGAGAGGGAGCTCC
AGCAGAGCCTGGAGCGTGAGAGGCTTTCTCTGGGGACCAGTGAGCTGGATATGGGGAAGGGCCCAATG
TATGATGTGGAGAAAAAGCCATGTATGTCTGGAGGCAGAAGGATGACTCTCAGAGATGACCAACCTGC
AAAGCTAGAAAAGGAGCCCAAGACGAGGCCAGAAGAGAACAAGCCAGAGCGGCCCAGCGGTCGGAAGC
CACGGCCCATGGGCATCATTGCCGCCAATGTGGAAAAGCATTATGAGACTGGCCGGGTCATTGGGGAT
GGGAACTTTGCTGTCGTGAAGGAGTGCAGACACCGCGAGACCAGGCAGGCCTATGCGATGAAGATCAT
TGACAAGTCCAGACTCAAGGGCAAGGAGGACATGGTGGACAGTGAGATCTTGATCATCCAGAGCCTCT
CTCACCCCAACATCGTGAAATTGCATGAAGTTTACGAAACAGACATGGAAATCTACCTGATCCTGGAG
TACGTGCAGGGAGGAGACCTTTTTGACGCCATCATAGAAAGTGTGAAGTTCCCGGAGCCCGATGCTGC
CCTCATGATCATGGACTTATGCAAAGCCCTCGTCCACATGCACGACAAGAGCATTGTCCACCGGGACC
TCAAGCCGGAAAACCTTTTGGTTCAGCGAAATGAGGACAAATCTACTACCTTGAAATTGGCTGATTTT
GGACTTGCAAAGCATGTGGTGAGACCTATATTTACTGTGTGTGGGACCCCAACTTACGTAGCTCCCGA
AATTCTTTCTGAGAAAGGTTATGGACTGGAGGTGGACATGTGGGCTGCTGGCGTGATCCTCTATATCC
TGCTGTGTGGCTTTCCCCCATTCCGCAGCCCTGAGAGGGACCAGGACGAGCTCTTTAACATCATCCAG
CTGGGCCACTTTGAGTTCCTCCCCCCTTACTGGGACAATATCTCTGATACAGCTGCTAAAGATCTGGT
GAGCCGGTTGCTGGTGGTAGACCCCAAAAAGCGCTACACAGCTCATCAGGTTCTTCAGCACCCCTGGA
TCGAAACAGCTGGCAAGACCAATACAGTGAAACGACAGAAGCAGGTGTCCCCCAGCAGCGAGGGTCAC
TTCCGGAGCCAGCACAAGAGGGTTGTGGAGCAGGTATCATAGTCACCACCTTGGGAATCTGTCCAGCC
CCCAGTTCTGCTCAAGGACAGAGAAAAGGATAGAAGTTTGAGAGAAAAACAATGAAAGAGGCTTCTTC
ACATAATTGGTGAATCAGAGGGAGAGACACTGAGTATATTTTAAAGCATATTA

NOV64j, CG57109-04

| | | | |
|---|---|---|---|
| Protein Sequence | SEQ ID NO: 974 | 744 aa | MW at 84729.4kD |

MLLIETYLRLGNLQRKVFLDLQLVPRGWGSLTIMAEGKEGQVPSYMDGSRQRENEEDAKAETPDVTIR
SYEIYSLPWNRQQGVCDHSLEYLSSRISERKLQGSWLPASRGNLEKPFLGPRGPVVPLFCPRNGLHSA
HPENSPLKPRVVTVVKLGGQRPRKITLLINRRSVQTFEQLLADISEALGSPRWKNDRVRKLFNLKGRE
IRSVSDFFREGDAFIAMGKEPLTLKSIQVAVEELYPNKARALTLAQHSRAPSPRLRSRLFSKALKGDH
RCGETETPKSCSEVAGCKAAMRHQGKIPEELSLDDRARTQKKWGRGKWEPEPSSKPPREATLEERHAR
GEKHLGVEIEKTSGEIIRCEKCKRERELQQSLERERLSLGTSELDMGKGPMYDVEKKPCMSGGRRMTL
RDDQPAKLEKEPKTRPEENKPERPSGRKPRPMGIEAANVEKHYETGRVIGDGNFAVVKECRHRETRQA
YAMKIIDKSRLKGKEDMVDSEILIIQSLSHPNIVKLHEVYETDMEIYLILEYVQGGDLFDAIIESVKF
PEPDAALMIMDLCKALVHMHDKSIVHRDLKPENLLVQRNEDKSTTLKLADFGLAKHVVRPIFTVCGTP
TYVAPEILSEKGYGLEVDMWAAGVILYILLCGFPPFRSPERDQDELFNIIQLGHFEFLPPYWDNISDT
AAKDLVSRLLVVDPKKRYTAHQVLQHPWIETAGKTNTVKRQKQVSPSSEGHFRSQHKRVVEQVS

| | | |
|---|---|---|
| NOV64k, CG57109-06 | SEQ ID NO: 975 | 2720 bp |

| | | |
|---|---|---|
| DNA Sequence | ORF Start: ATG at 149 | ORF Stop: TGA at 1826 |

GGACACTGACATGGACTGAAGGAGTAGAAAAGAAGCCTTGGGCTCTCCCAGATGGAAGAATGACCGTG
TGAGGAAACTGTTTAACCTCAAGGGCAGGGAAATCAGGAGCGTCTCTGATTTCTTCAGGGAAGGGGAT
GCTTTCATAGCTATGGGCAAAGAACCACTGACACTGAAGAGCATTCAGGTGGCTGTAGAAGAACTGTA
CCCCAACAAAGCCCGGGCCCTGACACTGGCCCAGCACAGCCGTGCCCCTTCTCCAAGGCTGAGGAGCA
GGCTGTTTAGCAAGGCTCTGAAAGGAGACCACCGCTGTGGGGAGACCGAGACCCCCAAGAGCTGCAGC
GAAGTTGCAGGATGCAAGGCAGCTATGAGGCACCAGGGGAAGATCCCCGAGGAGCTTTCACTAGATGA
CAGAGCGAGGACCCAGAAGAAGTGGGGGAGGGGGAAATGGGAGCCAGAACCCAGTAGCAAGCCCCCCA
GGGAAGCCACTCTGGAAGAGAGGCACGCAAGGGGAGAGAAGCATCTTGGGGTGGAGATTGAAAAGACC
TCGGGTGAAATTATCAGATGCGAGAAGTGCAAGAGAGAGAGGGAGCTTCAGCAGAGCCTGGAGCGTGA
GAGGCTTTCTCTGGGGACCAGTGAGCTGGATATGGGGAAGGGCCCAATGTATGATGTGGAGAAGCTGG
TGAGGACCAGAAGCTGCAGGAGGTCTCCCGAGGCAAATCCTGCAAGTGGGGAGGAAGGGTGGAAGGGT
GACAGCCACAGGAGCAGCCCCAGGAATCCCACTCAAGAGCTGAGGAGACCCAGCAAGAGCATGGACAA
GAAAGAGGACAGAGGCCCAGAGGATCAAGAAAGCCATGCTCAGGGAGCAGCCAAGGCCAAGAAGGACC
TTGTGGAAGTTCTTCCTGTCACAGAGGAGGGGCTGAGGGAGGTGAAGAAGGACACCAGGCCCATGAGC
AGGAGCAAACATGGTGGCTGGCTCCTGAGAGAGCACCAGGCGGGCTTTGAGAAGCTCCGCAGGACCCG
AGGAGAAGAGAAGGAGGCAGAGAAGGAGAAAAAGCCATGTATGTCTGGAGGCAGAAGGATGACTCTCA
GAGATGACCAACCTGCAAAGCTAGAAAAGGAGCCCAAGACGAGGCCAGAAGAGAACAAGCCAGAGCGG
CCCAGCGGTCGGAAGCCACGGCCCATGGGCATCATTGCCGCCAATGTGGAAAAGCATTATGAGACTGG
CCGGGTCATTGGGGATGGGAACTTTGCTGTCGTGAAGGAGTGCAGACACCGCGAGACCAGGCAGGCCT
ATGCGATGAAGATCATTGACAAGTCCAGACTCAAGGGCAAGGAGGACATGGTGGACAGTGAGATCTTG
CATGAAGTCTACGAAACAGACATGGAAATCTACCTGATCCTGGAGTACGTGCAGGGAGGAGACCTTTT
TGACGCCATCATAGAAAGTGTGAAGTTCCCGGAGCCCGATGCTGCCCTCATGATCATGGACTTATGCA
AAGCCCTCGTCCACATGCACGACAAGAGCATTGTCCACCGGGACCTCAAGCCGGAAAACCTTTTGGTT
CAGCGAAATGAGGACAAATCTACTACCTTGAAATTGGCTGATTTTGGACTTGCAAAGCATGTGGTGAG
ACCTATATTTACTGTGTGTGGGACCCCAACTTACGTAGCTCCCGAAATTCTTTCTGAGAAAGGTAAGT
GTTACACATCGATCTGTGGGACTCTAGTTCCCTTACTAACAAATGTTTCATTTGTCATATTTACTAGT
TTTCAATATGGAATAAATCTCAGAGAACTGACACTTAGGCTTGGATTTGGACTTCAATGAAAATATTT
GAAGTAGGCTTGACCAAAGCATGAGAGCTTTCTCCTCATTAGGGCTGCCCTTGTTACAGTCAATGGAT
CAGTGTGTGTGCATGTGTGTGTGTGTGTGTGTGTTTATTGTGTTTAGGCAGGACAGTGAGATGAAAGA
TGATGGAGAATTGGGTGGGGAACTCAAGCAAGCAAGGCTACTTGACCCAAGGCTATCTCTAATAGGAG
AGAATTGAAGCAGTCCTTATGGTACTTGGTTTAAAAATTTCTTCACCAACCTTGCATTTAAAGGAAAA
GGATCCCATTTTCCTCCATGAACTCTATGAATATTTATTACCTACTTGTATATTATGCAAGGATCCAA
TGAGCTGTTTTAGTGACAAACTTTCTAAAACATTTAAAAAGGAAATAATAGTTATGATATGGCTCTAA
ATATATGAATGACTATTTGACTACTGTGGCACTCCAGGAGAAACCAATTTACCCACCATTGGTAAGAT
GGGAAGACTCTCATTGGGTTGCAGGGTTGGTGACAGGGAGAAGGGATGGACGGATAGTTTCCCAGCAG
CAGAAGCATCAGGATAATTAAGATGAGGAGATGGCCAGGGATGGTGGCTCATGCCTGTAATCCCAGCT
CTTTGGGAGGCTGAGGCAGGTGGATCACCTGAGGTCAGGCGTTTGAAACCATCCTGGCCAACATGGTG
AAACCCTGTCTGTACTAAAACTACAAAAAAATTAGCTGGGCGTGGTGGCACATGCCTGTAATCTGAGC
TACTCGGGAGGCTGAGGCAGGAGAATTGCTTGAACCGGGGAGGTGGAGTTTGCAGTGAGCCAAGATCG
TGCCATTGCACTCCAGCCTGGGCAACAAGAGTGAAACTTCATCTCAAAAAAAAAAAAAAAAAAAAAAC

NOV64k, CG57109-06

| | | | |
|---|---|---|---|
| Protein Sequence | SEQ ID NO: 976 | 559 aa | MW at 63436.9kD |

MGKEPLTLKSIQVAVEELYPNKARALTLAQHSRAPSPRLRSRLFSKALKGDHRCGETETPKSCSEVAG
CKAAMRHQGKIPEELSLDDRARTQKKWGRGKWEPEPSSKPPREATLEERHARGEKHLGVEIEKTSGEI
IRCEKCKRERELQQSLERERLSLGTSELDMGKGPMYDVEKLVRTRSCRRSPEANPASGEEGWKGDSHR
SSPRNPTQELRRPSKSMDKKEDRGPEDQESHAQGAAKAKKDLVEVLPVTEEGLREVKKDTRPMSRSKH
GGWLLREHQAGFEKLRRTRGEEKEAEKEKKPCMSGGRRMTLRDDQPAKLEKEPKTRPEENKPERPSGR
KPRPMGIIAANVEKHYETGRVIGDGNFAVVKECRHRETRQAYAMKIIDKSRLKGKEDMVDSEILHEVY
ETDMEIYLILEYVQGGDLFDAIEESVKFPEPDAALMIMDLCKALVHMHDKSIVHRDLKPENLLVQRNE
DKSTTLKLADFGLAKHVVRPIFTVCGTPTYVAPEILSEKGKCYTSICGTLVPLLTNVSFVIFTSFQYG
INLRELTLRLGFGLQ

A ClustalW comparison of the above protein sequences yields the following sequence alignment shown in Table 64B.

TABLE 64B
Comparison of the NOV64 protein sequences.

| | |
|---|---|
| NOV64a | ------------------------------------------------------------ |
| NOV64b | ------------------------------------------------------------ |
| NOV64c | ------------------------------------------------------------ |
| NOV64d | ------------------------------------------------------------ |
| NOV64e | ------------------------------------------------------------ |
| NOV64f | ------------------------------------------------------------ |
| NOV64g | ------------------------------------------------------------ |
| NOV64h | ---------------------------------MAEGKEGQVPSYMDGSRQRENEEDAKA |
| NOV64i | MLLIETYLRLGNLQRKVFLDLQLVPRGWGSLTIMAEGKEGQVPSYMDGSRQRENEEDAKA |
| NOV64j | MLLIETYLRLGNLQRKVFLDLQLVPRGWGSLTIMAEGKEGQVPSYMDGSRQRENEEDAKA |
| NOV64k | ------------------------------------------------------------ |
| NOV64a | ------------------------------------------------------------ |
| NOV64b | ------------------------------------------------------------ |
| NOV64c | ------------------------------------------------------------ |
| NOV64d | ------------------------------------------------------------ |
| NOV64e | ------------------------------------------------------------ |
| NOV64f | ------------------------------------------------------------ |
| NOV64g | ------------------------------------------------------------ |
| NOV64h | ETPDVTIRSYEIYSLPWNRQQGVCDHSLEYLSSRISERKLQGSWLPASRGNLEKPFLGPR |
| NOV64i | ETPDVTIRSYEIYSLPWNRQQGVCDHSLEYLSSRISERKLQGSWLPASRGNLEKPFLGPR |
| NOV64j | ETPDVTIRSYEIYSLPWNRQQGVCDHSLEYLSSRISERKLQGSWLPASRGNLEKPFLGPR |
| NOV64k | ------------------------------------------------------------ |
| NOV64a | ------------------------------------------------------------ |
| NOV64b | ------------------------------------------------------------ |
| NOV64c | ------------------------------------------------------------ |
| NOV64d | ------------------------------------------------------------ |
| NOV64e | ------------------------------------------------------------ |
| NOV64f | ------------------------------------------------------------ |
| NOV64g | ------------------------------------------------------------ |
| NOV64h | GPVVPLFCPRNGLHSAHPENSPLKPRVVTVVKLGGQRPRKITLLLNRRSVQTFEQLLADI |
| NOV64i | GPVVPLFCPRNGLHSAHPENSPLKPRVVTVVKLGGQRPRKITLLLNRRSVQTFEQLLADI |
| NOV64j | GPVVPLFCPRNGLHSAHPENSPLKPRVVTVVKLGGQRPRKITLLLNRRSVQTFEQLLADI |
| NOV64k | ------------------------------------------------------------ |
| NOV64a | ----------------------------------------MGKEPLTLKSIQVAVEELYP |
| NOV64b | ------------------------------------------------------------ |
| NOV64c | ------------------------------------------------------------ |
| NOV64d | ----------------------------------------MGKEPLTLKSIQVAVEELYP |
| NOV64e | ------------------------------------TGSTMGKEPLTLKSIQVAVEELYP |
| NOV64f | ------------------------------------TGSTMGKEPLTLKSIQVAVEELYP |
| NOV64g | ------------------------------------------------------------ |
| NOV64h | SEALGSPRWKNDRVRKLFNLKGREIRSVSDFFREGDAFIAMGKEPLTLKSIQVAVEELYP |
| NOV64i | SEALGSPRWKNDRVRKLFNLKGREIRSVSDFFREGDAFIAMGKEPLTLKSIQVAVEELYP |
| NOV64j | SEALGSPRWKNDRVRKLFNLKGREIRSVSDFFREGDAFIAMGKEPLTLKSIQVAVEELYP |
| NOV64k | ----------------------------------------MGKEPLTLKSIQVAVEELYP |
| NOV64a | NKARALTLAQHSRAPSPRLRSRLFSKALKGDHRCGETETPKSCSEVAGCKAAMRHQGKIP |
| NOV64b | ------------------------------------------------------------ |
| NOV64c | ------------------------------------------------------------ |
| NOV64d | NKARALTLAQHSRAPSPRLRSRLFSKALKGDNRCGETETPKSCSEVAGCKAAMRHQGKIP |
| NOV64e | NKARALTLAQHSRAPSPRLRSRLFSKALKGDHRCGETETPKSCSEVAGCKAANRHQGKIP |
| NOV64f | NKARALTLAQHSRAPSPRLRSRLFSKALKGDHRCGETETPKSCSEVAGCKAAMRHQGKIP |
| NOV64g | ------------------------------------------------------------ |
| NOV64h | NKARALTLAQHSRAPSPRLRSRLFSKALKGDHRCGETETPKSCSEVAGCKAAMRHQGKIP |
| NOV64i | NKARALTLAQHSRAPSPRLRSRLFSKALKGDHRCGETETPKSCSEVAGCKAANRHQGKIP |
| NOV64j | NKARALTLAQHSRAPSPRLRSRLFSKALKGDHRCGETETPKSCSEVAGCKAANRHQGKIP |
| NOV64k | NKARALTLAQHSRAPSPRLRSRLFSKALKGDHRCGETETPKSCSEVAGCKAANRHQGKIP |
| NOV64a | EELSLDDRARTQKKWGRGKWEPEPSSKPPREATLEERHARGEKHLGVEIEKTSGEIIRCE |
| NOV64b | ------------------------------------------------------------ |
| NOV64c | ------------------------------------------------------------ |
| NOV64d | EELSLDDRARTQKKWGRGKWEPEPSSKPPREATLEERHARGEKHLGVEIEKTSGEIIRCE |
| NOV64e | EELSLDDRARTQKKWGRGKWEPEPSSKPPREATLEERHARGEKHLGVEIEKTSGEIIRCE |
| NOV64f | EELSLDDRARTQKKWGRGKWEPEPSSKPPREATLEERHARGEKHLGVEIEKTSGEIIRCE |
| NOV64g | ------------------------------------------------------------ |
| NOV64h | EELSLDDRARTQKKWGRGKWEPEPSSKPPREATLEERHARGEKHLGVEIEKTSGEIIRCE |
| NOV64i | EELSLDDRARTQKKWGRGKWEPEPSSKPPREATLEERHARGEKHLGVEIEKTSGEIIRCE |
| NOV64j | EELSLDDRARTQKKWGRGKWEPEPSSKPPREATLEERHARGEKHLGVEIEKTSGEIIRCE |
| NOV64k | EELSLDDRARTQKKWGRGKWEPEPSSKPPREATLEERHARGEKHLGVEIEKTSGEIIRCE |
| NOV64a | KCKRERELQQSLERERLSLGTSELDMGKGPMYDVEKLVRTRSCRRSPEANPASGEEGWKG |
| NOV64b | ------------------------------------------------------------ |
| NOV64c | ------------------------------------------------------------ |
| NOV64d | KCKRERELQQSLERERLSLGTSELDMGKGPMYDVEKLVRTRSCRRSPEANPASGEEGWKG |
| NOV64e | KCKRERELQQSLERERLSLGTSELDMGKGPMYDVEKLVRTRSCRRSPEANPASGEEGWKG |
| NOV64f | KCKRERELQQSLERERLSLGTSELDNGKGPMYDVEKLVRTRSCRRSPEANPASGEEGWKG |
| NOV64g | ------------------------------------------------------------ |
| NOV64h | KCKRERELQQSLERERLSLGTSELDNGKGPMYDVEKLVRTRSCRRSPEANPASGEEGWKG |
| NOV64i | KCKRERELQQSLERERLSLGTSELDMGKGPMYDVEKLVRTRSCRRSPEANPASGEEGWKG |
| NOV64j | KCKRERELQQSLERERLSLGTSELDMGKGPMYDVE------------------------- |
| NOV64k | KCKRERELQQSLERERLSLGTSELDMGKGPMYDVEKLVRTRSCRRSPEANPASGEEGWKG |
| NOV64a | DSHRSSPRNPTQELRRPSKSMDKKEDRGPEDQESHAQGAAKAKKDLVEVLPVTEEGLREV |
| NOV64b | ------------------------------------------------------------ |
| NOV64c | ------------------------------------------------------------ |
| NOV64d | DSHRSSPRNPTQELRRPSKSMDKKEDRGPEDQESHAQGAAKAKKDLVEVLPVTEEGLREV |
| NOV64e | DSHRSSPRNPTQELRRPSKSMDKKEDRGPEDQESHAQGAAKAKKDLVEVLPVTEEGLREV |
| NOV64f | DSHRSSPRNPTQELRRPSKSMDKKEDRGPEDQESHAQGAAKAKKDLVEVLPVTEEGLREV |
| NOV64g | ------------------------------------------------------------ |
| NOV64h | DSHRSSPRNPTQELRRPSKSMDKKEDRGPEDQESHAQGAAKAKKDLVEVLPVTEEGLREV |
| NOV64i | DSHRSSPRNPTQELRRPSKSMDKKEDRGPEDQESHAQGAAKAKKDLVEVLPVTEEGLREV |
| NOV64j | ------------------------------------------------------------ |
| NOV64k | DSHRSSPRNPTQELRRPSKSMDKKEDRGPEDQESHAQGAAKAKKDLVEVLPVTEEGLREV |
| NOV64a | KKDTRPMSRSKHGGWLLREHQAGFEKLRRTRGEEKEAEKEKKPCMSGGRRNTLRDDQPAK |
| NOV64b | ------------------------------------------------------------ |
| NOV64c | ------------------------------------------------------------ |
| NOV64d | KKDTRPMSRSKHGGWLLREHQAGFEKLRRTRGEEKEAEKEKKPCMSGGRRMTLRDDQPAK |
| NOV64e | KKDTRPMSRSKHGGWLLREHQAGFEKLRRTRGEEKEAEKEKKPCMSGGRRMTLRDDQPAK |
| NOV64f | KKDTRPMSRSKHGGWLLREHQAGFEKLRRTRGEEKEAEKEKKPCMSGGRRMTLRDDQPAK |
| NOV64g | ------------------------------------------------------------ |
| NOV64h | KKDTRPMSRSKHGGWLLREHQAGFEKLRRTRGEEKEAEKEKKPCMSGGRRMTLRDDQPAK |
| NOV64i | KKDTRPMSRSKHGGWLLREHQAGFEKLRRTRGEEKEAEKEKKPCMSGGRRMTLRDDQPAK |
| NOV64j | ----------------------------------------KKPCMSGGRRMTLRDDQPAK |
| NOV64k | KKDTRPMSRSKHGGWLLREHQAGFEKLRRTRGEEKEAEKEKKPCMSGGRRNTLRDDQPAK |
| NOV64a | LEKEPKTRPEENKPERPSGRKPRPMGIIAANVEKHYETGRVIGDGNFAVVKECRHRETRQ |
| NOV64b | --------------------------------TGSYETGRVIGDGNFAVVKECRHRETRQ |
| NOV64c | --------------------------------TGSYETGRVIGDGNFAVVKECRHRETRQ |
| NOV64d | LEKEPKTRPEENKPERPSGRKPRPMGIIAANVEKHYETGRVIGDGNFAVVKECRHRETRQ |
| NOV64e | LEKEPKTRPEENKPERPSGRKPRPMGIIAANVEKHYETGRVIGDGNFAVVKECRHRETRQ |
| NOV64f | LEKEPKTRPEENKPERPSGRKPRPMGIIAANVEKHYETGRVIGDGNFAVVKECRHRETRQ |
| NOV64g | --------------------------------TGSYETGRVIGDGNFAVVKECRHRETRQ |
| NOV64h | LEKEPKTRPEENKPERPSGRKPRPMGIIAANVEKHYETGRVIGDGNFAWKECRHRETRQ |
| NOV64i | LEKEPKTRPEENKPERPSGRKPRPMGIIAANVEKHYETGRVIGDGNFAVVKECRHRETRQ |
| NOV64j | LEKEPKTRPEENKPERPSGRKPRPMGIIAANVEKHYETGRVIGDGNFAVVKECRHRETRQ |
| NOV64k | LEKEPKTRPEENKPERPSGRKPRPMGIIAANVEKHYETGRVIGDGNFAVVKECRHRETRQ |
| NOV64a | AYAMKIIDKSRLKGKEDMVDSEILIIQSLSHPNIVKLHEVYETDMEIYLILEYVQGGDLF |
| NOV64b | AYANKIIDKSRLKGKEDMVDSEILIIQSLSHPNIVKLHEVYETDMEIYLILEYVQGGDLF |
| NOV64c | AYAMKIIDKSRLKGKEDMVDSEILIIQSLSHPNIVKLHEVYETDMEIYLILEYVQGGDLF |
| NOV64d | AYAMKIIDKSRLKGKEDMVDSEILIIQSLSHPNIVKLHEVYETDMEIYLILEYVQGGDLF |
| NOV64e | AYAMKIIDKSRLKGKEDMVDSEILIIQSLSHPNIVKLHEVYETDMEIYLILEYVQGGDLF |
| NOV64f | AYAMKIIDKSRLKGKEDMVDSEILIIQSLSHPNIVKLHEVYETDMEIYLILEYVQGGDLF |
| NOV64g | AYAMKIIDKSRLKGKEDMVDSEILIIQSLSHPNIVKLHEVYETDMEIYLILEYVQGGDLF |
| NOV64h | AYAMKIIDKSRLKGKEDMVDSEILIIQSLSHPNIVKLHEVYETDMEIYLILEYVQGGDLF |
| NOV64i | AYAMKIIDKSRLKGKEDMVDSEI-------------LHEVYETDMEIYLILEYVQGGDLF |
| NOV64j | AYAMKIIDKSRLKGKEDMVDSEILIIQSLSHPNIVKLHEVYETDMEIYLILEYVQGGDLF |
| NOV64k | AYANKIIDKSRLKGKEDMVDSEIL-------------HEVYETDMEIYLILEYVQGGDLF |
| NOV64a | DAIIESVKFPEPDAALMIMDLCKALVHMHDKSIVHRDLKPENLLVQRNEDKSTTLKLADF |
| NOV64b | DAIIESVKFPEPDAALMIMDLCKALVHMHDKSIVHRDLKPENLLVQRNEDKSTTLKLADF |
| NOV64c | DAIIESVKFPEPDAALMIMDLCKALVHMHDKSIVHRDLKPENLLVQRNEDKSTTLKLADF |
| NOV64d | DAIIESVKFPEPDAALMIMDLCKALVHMHDKSIVHRDLKPENLLVQRNEDKSTTLKLADF |
| NOV64e | DAIIESVKFPEPDAALMIMDLCKALVHMHDKSIVHRDLKPENLLVQRNEDKSTTLKLADF |
| NOV64f | DAIIESVKFPEPDAALMIMDLCKALVHMHDKSIVHRDLKPENLLVQRNEDKSTTLKLADF |
| NOV64g | DAIIESVKFPEPDAALMIMDLCKALVHMHDKSIVHRDLKPENLLVQRNEDKSTTLKLADF |
| WOV64h | DAIIESVKFPEPDAALMIMDLCKALVHMHDKSIVHRDLKPENLLVQRNEDKSTTLKLADF |
| NOV64i | DAIIESVKFPEPDAALMIMDLCKALVHMHDKSIVHRDLKPENLLVQRNEDKSTTLKLADF |
| NOV64j | DAIIESVKFPEPDAALMIMDLCKALVHMHDKSIVHRDLKPENLLVQRNEDKSTTLKLADF |
| NOV64k | DAIIESVKFPEPDAALMIMDLCKALVHMHDKSIVHRDLKPENLLVQRNEDKSTTLKLADF |
| NOV64a | GLAKHVVRPIFTVCGTPTYVAPEILSEKGYGLEVDMWAAGVILYILLCGFPPFRSPERDQ |
| NOV64b | GLAKHVVRPIFTVCGTPTYVAPEILSEKGYGLEVDMWAAGVILYILLCGFPPFRSPERDQ |
| NOV64c | GLAKHVVRPIFTVCGTPTYVAPEILSEK-------------------------------- |
| NOV64d | GLAKHVVRPIFTVCGTPTYVAPEILSEKGYGLEVDMWAAGVILYILLCGFPPFRSPERDQ |
| NOV64e | GLAKHVVRPIFTVCGTPTYVAPEILSEKGYGLEVDMWAAGVILYILLCGFPPFRSPERDQ |
| NOV64f | GLAKHVVRPIFTVCGTPTYVAPEILSEKG------------------------------- |
| NOV64g | GLAKHVVRPIFTVCGTPTYVAPEILSEKGYGLEVDMWAAGVILYILLCGFPPFRSPERDQ |
| NOV64h | GLAKHVVRPIFTVCGTPTYVAPEILSEKGYGLEVDMWAAGVILYILLCGFPPFRSPERDQ |
| NOV64i | GLAKHVVRPIFTVCGTPTYVAPEILSEKGYGLEVDMWAAGVILYILLCGFPPFRSPERDQ |
| NOV64j | GLAKHVVRPIFTVCGTPTYVAPEILSEKGYGLEVDMWAAGVILYILLCGFPPFRSPERDQ |
| NOV64a | DELFNIIQLGHFEFLPPYWDNISD-AAKDLVSRLLVVDPKKRYTAHQVLQHPWIETAGKT |
| NOV64b | DELFNIIQLGHFEFLPPYWDNISD-AAKDLVSRLLVVDPKKRYTAHQVLQHPWILEG--- |
| NOV64c | ---------------------AAK----DLVSRLLVVDPKKRYTAHQVLQHPWILEG--- |
| NOV64d | DELFNIIQLGHFEFLPPYWDNISD-AAKDLVSRLLVVDPKKRYTAHQVLQHPWIETAGKT |
| NOV64e | DELFNIIQLGHFEFLPPYWDNISD-AAKDLVSRLLVVDPKKRYTAHQVLQHPWIETAGKT |
| NOV64f | -------------------------------YGLELEG---------------------- |
| NOV64g | DELFNIIQLGHFEFLPPYWDNISD-AAKDLVSRLLVVDPKKRYTAHQVLQHPWILEG--- |
| NOV64h | DELFNIIQLGHFEFLPPYWDNISDTAAKDLVSRLLVVDPKKRYTAHQVLQHPWIETAGKT |
| NOV64i | DELFNIIQLGHFEFLPPYWDNISD-AAKDLVSRLLVVDPKKRYTAHQVLQHPWIETAGKT |
| NOV64j | DELFNIIQLGHFEFLPPYWDNISDTAAKDLVSRLLVVDPKKRYTAHQVLQHPWIETAGKT |
| NOV64k | --------T-----LVPLLTNVSFVIFTSFQYGINLRELTLRLGFGLQ------------ |
| NOV64a | NTVKRQKQVSPSSEGHFRSQHKRVVEQVS--- |
| NOV64b | -------------------------------- |
| NOV64c | -------------------------------- |
| NOV64d | NTVKRQKQVSPSSEGHFRSQHKRVVEQVS--- |
| NOV64e | NTVKRQKQVSPSSEGHFRSQHKRVVEQVSLEG |
| NOV64f | -------------------------------- |
| NOV64g | -------------------------------- |
| NOV64h | NTVKRQKQVSPSSEGHFRSQHKRVVEQVS--- |
| NOV64i | NTVKRQKQVSPSSEGHFRSQHKRVVEQVS--- |
| NOV64j | NTVKRQKQVSPSSEGHFRSQHKRVVEQVS--- |
| NOV64k | -------------------------------- |
| NOV64a | (SEQ ID NO: 956) |
| NOV64b | (SEQ ID NO: 958) |
| NOV64c | (SEQ ID NO: 960) |
| NOV64d | (SEQ ID NO: 962) |
| NOV64e | (SEQ ID NO: 964) |
| NOV64f | (SEQ ID NO: 966) |
| NOV64g | (SEQ ID NO: 968) |
| NOV64h | (SEQ ID NO: 970) |
| NOV64i | (SEQ ID NO: 972) |
| NOV64j | (SEQ ID NO: 974) |
| NOV64k | (SEQ ID NO: 976) |

Further analysis of the NOV64a protein yielded the following properties shown in Table 64C.

TABLE 64C
Protein Sequence Properties NOV64a

| | |
|---|---|
| SignalP analysis: | No Known Signal Sequence Indicated |
| PSORT II analysis: | |

```
PSG: a new signal peptide prediction method
    N-region: length 9; pos.chg 2; neg.chg 1
    H-region: length 6; peak value −5.22
    PSG score: −9.62
GvH: von Heijne's method for signal seq. recognition
    GvH score (threshold: −2.1): −7.85
    possible cleavage site: between 34 and 35
>>> Seems to have no N-terminal signal peptide
ALOM: Klein et al's method for TM region allocation
    Init position for calculation: 1
    Tentative number of TMS(s) for the threshold 0.5: 1
    Number of TMS(s) for threshold 0.5: 1
```

| | | |
|---|---|---|
| INTEGRAL | Likelihood = −3.29 | Transmembrane 534-550 |

| | |
|---|---|
| PERIPHERAL | Likelihood = 4.72 (at 452) |

```
    ALOM score: −3.29 (number of TMSs: 1)
MTOP: Prediction of membrane topology (Hartmann et al.)
    Center position for calculation: 541
    Charge difference: 1.0 C(−2.0) − N(−3.0)
    C > N: C-terminal side will be inside
>>> Single TMS is located near the C-terminus
>>> membrane topology: type Nt (cytoplasmic tail 1 to 533)
MITDISC: discrimination of mitochondrial targeting seq
```

| | | | |
|---|---|---|---|
| R content: | 0 | Hyd Moment(75): | 9.28 |
| Hyd Moment(95): | 7.07 | G content: | 1 |
| D/E content: | 2 | S/T content: | 2 |
| Score: −6.37 | | | |

```
Gavel: prediction of cleavage sites for mitochondrial preseq
    R-10 motif at 52 SRL FS
NUCDISC: discrimination of nuclear localization signals
    pat4: RKPR (4) at 340
    pat4: PKKR (4) at 598
    pat7: PSGRKPR (3) at 337
    pat7: PKKRYTA (5) at 598
    bipartite: none
    content of basic residues: 17.9%
    NLS Score: 0.72
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
    type 1: none
    type 2: none
NMYR: N-myristoylation pattern: none
Prenylation motif: none
memYQRL: transport motif from cell surface to Golgi: none
Tyrosines in the tail: too long tail
Dileucine motif in the tail: found
    LL at 276
    LL at 483
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
    Prediction: nuclear
    Reliability: 89
COIL: Lupas's algorithm to detect coiled-coil regions
    total: 0 residues
Final Results (k = 9/23):
    30.4%: nuclear
    26.1%: cytoplasmic
    13.0%: Golgi
      8.7%: mitochondrial
      8.7%: vesicles of secretory system
      8.7%: endoplasmic reticulum
      4.3%: peroxisomal
>> prediction for CG57109-01 is nuc (k = 23)
```

A search of the NOV64a protein against the Geneseq database, a proprietary database that contains sequences published in patents and patent publication, yielded several homologous proteins shown in Table 64D.

TABLE 64D
Geneseq Results for NOV64a

| | | NOV64a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE24133 | Human kinase (PKIN)-4 protein - | 1 . . . 648 | 648/648 (100%) | 0.0 |
| | Homo sapiens, 835 aa. | 188 . . . 835 | 648/648 (100%) | |
| | [WO200233099-A2, 25-APR- | | | |
| | 2002] | | | |
| ABB82471 | Human serine/threonine protein | 1 . . . 648 | 648/648 (100%) | 0.0 |
| | kinase - Homo sapiens, 835 aa. | 188 . . . 835 | 648/648 (100%) | |
| | [WO200270678-A2, 12-SEP- | | | |
| | 2002] | | | |
| AAO15419 | Novel human kinase protein 2 - | 1 . . . 648 | 647/648 (99%) | 0.0 |
| | Homo sapiens, 817 aa. | 170 . . . 817 | 648/648 (99%) | |
| | [WO200242438-A2, 30-MAY- | | | |
| | 2002] | | | |
| ABB82474 | Human serine/threonine protein | 1 . . . 484 | 484/484 (100%) | 0.0 |
| | kinase fragment - Homo sapiens, | 188 . . . 671 | 484/484 (100%) | |
| | 751 aa. [WO200270678-A2, 12- | | | |
| | SEP-2002] | | | |
| AAY42696 | Rat serine-threonine protein | 1 . . . 648 | 495/649 (76%) | 0.0 |
| | kinase PK80 sequence - Rattus | 98 . . . 733 | 535/649 (82%) | |
| | norvegicus, 733 aa. | | | |
| | [WO9950395-A1, 07-OCT- | | | |
| | 1999] | | | |

In a BLAST search of public sequence databases, the NOV64a protein was found to have homology to the proteins shown in the BLASTP data in Table 64E.

TABLE 64E
Public BLASTP Results for NOV64a

| | | NOV64a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9C098 | Hypothetical protein KIAA1765 - | 41 . . . 648 | 608/608 (100%) | 0.0 |
| | Homo sapiens (Human), 608 aa | 1 . . . 608 | 608/608 (100%) | |
| | (fragment). | | | |
| Q8BWQ5 | Hypothetical eukaryotic protein | 1 . . . 632 | 467/633 (73%) | 0.0 |
| | kinase containing protein - Mus | 1 . . . 619 | 510/633 (79%) | |
| | musculus (Mouse), 619 aa. | | | |
| O15075 | Serine/threonine-protein kinase | 348 . . . 641 | 155/294 (52%) | 3e−87 |
| | DCAMKL1 (EC 2.7.1.-) | 382 . . . 674 | 212/294 (71%) | |
| | (Doublecortin-like and CAM | | | |
| | kinase-like 1) - Homo sapiens | | | |
| | (Human), 740 aa. | | | |
| Q8BQN2 | Double cortin and | 348 . . . 641 | 154/294 (52%) | 1e−86 |
| | calcium/calmodulin-dependent | 75 . . . 367 | 212/294 (71%) | |
| | protein kinase-like 1 - Mus | | | |
| | musculus (Mouse), 452 aa. | | | |
| Q8CCN4 | Double cortin and | 348 . . . 641 | 154/294 (52%) | 1e−86 |
| | calcium/calmodulin-dependent | 91 . . . 383 | 212/294 (71%) | |
| | protein kinase-like 1 - Mus | | | |
| | musculus (Mouse), 449 aa. | | | |

PFam analysis indicates that the NOV64a protein contains the domains shown in the Table 64F.

TABLE 64F
Domain Analysis of NOV64a

| | | Identities/ | |
|---|---|---|---|
| | NOV64a | Similarities | |
| | Match | for the | |
| Pfam Domain | Region | Matched Region | Expect Value |
| pkinase | 356 . . . 613 | 109/301 (36%) | 8.1e-102 |
| | | 220/301 (73%) | |

### Example 65

The NOV65 clone was analyzed, and the nucleotide and encoded polypeptide sequences are shown in Table 65A.

TABLE 65A
NOV65 Sequence Analysis

| | | |
|---|---|---|
| NOV65a, CG57399-04 | SEQ ID NO: 977 | 947 bp |

| | | |
|---|---|---|
| DNA Sequence | ORF Start: ATG at 7 | ORF Stop: TGA at 937 |

GACCTGATGAAGAATGACACGAGGATACACTTTCAGGAAGACTGGAAGATAATAACCCTGTTTATAGG
CGGCAATGACCTCTGTGATTTCTGCAATGATCTGGTCCACTATTCTCCCCAGAACTTCACAGACAACA
TTGGAAAGGCCCTGGACATCCTCCATGCTGAGGTTCCTCGGGCATTTGTGAACCTGGTGACGGTGCTT
GAGATCGTCAACCTGAGGGAGCTGTACCAGGAGAAAAAAGTCTACTGCCCAAGGATGATCCTCAGGTC
TCTGTGTCCCTGTGTCCTGAAGTTTGATGATAACTCAACAGAACTTGCTACCCTCATCGAATTCAACA
AGAAGTTTCAGGAGAAGACCCACCAACTGATTGAGAGTGGGCGATATGACACAAGGGAAGATTTTACT
GTGGTTGTGCGGCCGTTCTTTGAAAACGTGGACATGCCAAAGACCTCGGAAGGATTGCCTGACAACTC
TTTCTTCGCTCCTGACTGTTTCCACTTCAGCAGCAAGTCTCACTCCCGAGCAGCCAGTGCTCTCTGGA
ACAATATGCTGGAGCCTGTTGGCCAGAAGACGACTCGTCATAAGTTTGAAAACAAGATCAATATCACA
TGTCCGAACCAGGTCCAGCCGTTTCTGAGGACCTACAAGAACAGCATGCAGGGTCATGGGACCTGGCT
GCCATGCAGGGACAGAGCCCCTTCTGCCTTGCACCCTACCTCAGTGCATGCCCTGAGACCTGCAGACA
TCCAAGTTGTGGCTGCTCTGGGGGATTCTCTGACCGCTGGCAATGGAATTGGCTCCAAACCAGACGAC
CTCCCCGATGTCACCACACAGTATCGGGGACTGTCATACAGAGAAAGTAAACCAGGGTTCTTATCAGA
CTCCTGGGTCAGCAAATCCAACAGGAAATGCACCAGAAAAGCACCAAATCCCTGAATCTACAC

| | |
|---|---|
| NOV65a, CG57399-04 | +TL,14 |

| | | | |
|---|---|---|---|
| Protein Sequence | SEQ ID NO: 978 | 310 aa | MW at 35268.7kD |

MKNDTRIHFQEDWKIITLFIGGNDLCDFCNDLVHYSPQNFTDNIGKALDILHAEVPRAFVNLVTVLEI
VNLRELYQEKKVYCPRMILRSLCPCVLKFDDNSTELATLIEFNKKFQEKTHQLIESGRYDTREDFTVV
VRPFFENVDMPKTSEGLPDNSFFAPDCFHFSSKSHSRAASALWNNMLEPVGQKTTRHKFENKINITCP
NQVQPFLRTYKNSMQGHGTWLPCRDRAPSALHPTSVHALRPADIQVVAALGDSLTAGNGIGSKPDDLP
DVTTQYRGLSYRESKPGFLSDSWVSKSNRKCTRKAPNP

| | | |
|---|---|---|
| NOV65b, CG57399-01 | SEQ ID NO: 979 | 4268 bp |

| | | |
|---|---|---|
| DNA Sequence | ORF Start: ATG at 1 | ORF Stop: TAG at 4258 |

ATGACCTGGGACACAGCTCTCTGGACCTCAGTTTTTCTGATTGGGCTCCTTCCTACCCTTGGTTTCGC
TAATTGCATCCTCCAGACTTCTGGTAAAATGTGTACTTTAAGAGGTAGATACCCCCAGCCCCCACAAC
CACCTCTCTGCTTGTCTCCCCTAGTCCACCAGCTCCGACCAGCAGACATCAAAGTGGTGGCCGCCCTG
GGTAATGATGAAACCTTCCAGGAAAGTGGTGCAGGGCAGCTAAGTGAGCCTGACCCCAGGCAGTGGTC
CTGGCCACAGGCCTGCTTGCCTGGGGTAAAAAAGGAAATGCAAGATGTGGTAGGTGAGAGAACGCCGA
GCCGTCGCCGCAGCCTCCGCCGCCGAGAAGCCCTTGTTCCCGCTGCTGGGAAGGAGAGTCTGTGCCGA
CAAGATATTTTCATTTCCTTGTTGGAAATTATCAAGCATTTTCCTCCCTCCCCTCAGGACATCAACCT
GGAGAAAGACTGGAAGCTGGTCACACTCTTCATTGGGGTCAACGACTTGTGTCATTACTGTCCACTTG
TTCAGGGCCCCGTTATAGACCTGGGTGGGATGGATACCCTCCACTCCCTGCAGCTCCCAAGGGCTTTC
GTCAACGTGGTGGAGGTCATGGAGCTGGCTAGCCTGTACCAGGGCCAAGGCGGGAAATGTGCCATGCT
GGCAGCTCAGGAAGCCTGGAACAGCCTCCTGGCCTCCAGCAGGTACAGTGAGCAGGAGTCCTTCACCG
TGGTTTTCCAGCCTTTCTTCTATGAGACCACCCCATCTGACCCCCGACTCCAGGATTCTACCACGCTG
GCCTGGCATCTCTGGAATAGGATGATGGAGCCAGCAGGAGAGAAAGATGAGCCATTGAGTGTAAAACA
CGGGAGGCCAATGAAGTGTCCCTCTCAGGAGAGCCCCTATCTGTTCAGCTACAGAAACAGCAACTACC
TGACCAGACTGCAGAAACCCCAAGACAAGCTTGTAAGAGAAGGAGCGGAAATCAGATGTCCTGACAAA
GACCCCTCCGATACGGTTCCCACCTCAGTTCATAGGCTGAAGCCGGCTGACATCAACGTAATTGGAGC
CCTGGGTGACTCTCTCACGGCAGGCAATGGGGCCGGGTCCACACCTGGGAACGTCTTGGACGTCTTGA
CTCAGTACCGAGGCCTGTCCTGGAGCGTCGGCGGAGATGAGAACATCGGCACCGTTACCACCCTGGCA
GACATCCTCCGGGAATTCAACCCTTCCCTGAAGGGCTTCTCTGTTGGCACTGGGAAAGAAACCAGTCC
TAATGCCTTCTTAAACCAGGCTGTGGCAGGAGGCCGAGCTGAGCAGGCCAGGAGGCTGGTGGACCTGA
TGAAGAATGACACGAGGATACACTTTCAGGAAGACTGGAAGATAATAACCCTGTTTATAGGCGGCAAT
GACCTCTGTGATTTCTGCAATGATCTGGTACACTATTCTCCCCAGAACTTCACAGACAACATTGGAAA
GGCCCTGGACATCCTCCATGCTGAGTCTCAGGTTCCTCGGGCATTTGTGAACCTGGTGACGGTGCTTG
AGATCGTCAACCTGAGGGAGCTGTACCAGGAGAAAAAAGTCTACTGCCCAAGGATGATCCTCAGGTCA
CTGTGTCCCTGTGTCCTGAAGTTTGATGATAACTCAACAGAACTTGCTACCCTCATCGAATTCAACAA
GAAGTTTCAGGAGAAGACCCACCAACTGATTGAGAGTGGGCGATATGACACAAGGGAAGATTTTACTG
TGGTTGTGCAGCCGTTCTTTGAAAACGTGGACATGCCAAAGACCCAGGAAGGATTGCCTGACAACTCT
TTCTTCGCTCCTGACTGTTTCCACTTCAGCAGCAAGTCTCACTCCCGAGCAGCCAGTGCTCTCTGGAA
CAATATGCTGGAGCCTGTTGGCCAGAAGACGACTCGTCATAAGTTTGAAAACAAGATCAATATCACAT
GTCCGTCACAGGTCCAGCCGTTTCTGAGGACCTACAAGAACAGCATGCAGGGTCATGGGACCTGGCTG
CCATGCAGGGACAGAGCCCCTTCTGCCTTGCACCCTACCTCAGTGCATGCCCTGAGACCTGCAGACAT
CCAAGTTGTGGCTGCTCTGGGGGATTCTCTGACCGCTGGCAATGGAATTGGCTCCAAACCAGACGACC
TCCCCGATGTCACCACACAGTATCGGGGACTGTCATACAGTGCAGGAGGGGACGGCTCCCTGGAGAAT
GTGACCACCTTACCTAGTTCTATCCTTCGGGAGTTTAACAGAAACCTCACAGGCTACGCCGTGGGCAC
GGGTGATGCCAATGACACGAATGCATTCCTCAATCAAGCTGTTCCCGGAGCAAAGGCTAGGGATCTTA
TGAGCCAAGTCCAAACTCTGATGCAGAAGATGAAAGATGATCATAGAGTAAATTTCCATGAAGACTGG
AAGGTCATCACAGTGCTGATCGGAGGCAGCGATTTATGTGACTACTGCACAGATTCGAATCTGTATTC
TGCAGCCAACTTTGTTCACCATCTCCGCAATGCCTTGGACGTCCTGCATAGAGAGGTGCCCAGAGTCC
TGGTCAACCTCGTGGACTTCCTGAACCCCACTATCATGCGGCAGGTGTTCCTGGGAAACCCAGACAAG
TGCCCAGTGCAGCAGGCCAGCGTTTTGTGTAACTGCGTTCTGACCCTGCGGGAGAACTCCCAAGAGCT
AGCCAGGCTGGAGGCCTTCAGCCGAGCCTACCAGAGCAGCATGCGCGAGCTGGTGGGGTCAGGCCGCT
ATGACACGCAGGAGGACTTCTCTGTGGTGCTGCAGCCCTTCTTCCAGAACATCCAGCTCCCTGTCCTG
CAGGATGGGCTCCCAGATACGTCCTTCTTTGCCCCAGACTGCATCCACCCAAATCAGAAATTCCACTC
CCAGCTGGCCAGAGCCCTTTGGACCAATATGCTTGAACCACTTGGAAGCAAAACAGAGACCCTGGACC
TGAGAGCAGAGATGCCCATCACCTGTCCCACTCAGAATGAGCCCTTCCTGAGAACCCCTCGGAATAGT
AACTACACGTACCCCATCAAGCCAGCCATTGAGAACTGGGGCAGTGACTTCCTGTGTACAGAGTGGAA
GGCTTCCAATAGTGTTCCAACCTCTGTCCACCAGCTCCGACCAGCAGACATCAAAGTGGTGGCCGCCC
TGGGTGACTCTCTGACTACAGCAGTGGGAGCTCGACCAAACAACTCCAGTGACCTACCCACATCTTGG
AGGGGACTCTCTTGGAGCATTGGAGGGGATGGGAACTTGGAGACTCACACCACACTGCCCAGTATTCT
GAAGAAGTTCAACCCTTACCTCCTTGGCTTCTCTACCAGCACCTGGGAGGGGACAGCAGGACTAAATG
TGGCAGCGGAAGGGGCCAGAGCTAGGAGGGACATGCCAGCCCAGGCCTGGGACCTGGTAGAGCGAATG
AAAAACAGCCCCATACACTTTCAGGAAGACTGGAAGATAATAACCCTGTTTATAGGCGGCAATGACCT
CTGTGATTTCTGCAATGATCTGGTAGGTGAATATGTTCAGCACATCCAACAGGCCCTGGACATCCTCT
CTGAGGAGCTCCCAAGGGCTTTCGTCAACGTGGTGGAGGTCATGGAGCTGGCTAGCCTGTACCAGGGC
CAAGGCGGGAAATGTGCCATGCTGGCAGCTCAGAACAACTGCACTTGCCTCAGACACTCGCAAAGCTC
CCTGGAGAAGCAAGAACTGAAGAAAGTGAACTGGAACCTCCAGCATGGCATCTCCAGTTTCTCCTACT
GGCACCAATACACACAGCGTGAGGACTTTGCGGTTGTGGTGCAGCCTTTCTTCCAAAACACACTCACC
CCACTGAACAGAGGGGACACTGACCTCACCTTCTTCTCCGAGGACTGTTTTCACTTCTCAGACCGCGG
GCATGCCGAGATGGCCATCGCACTCTGGAACAACATGCTGGAACCAGTGGGCCGCAAGACTACCTCCA
ACAACTTCACCCACAGCCGAGCCAAACTCAAGTGCCCCTCTCCTGTGAGTCCTTACCTCTACACCCTG
CGGAACAGCCGATTGCTCCCAGACCAGGCTGAAGAAGCCCCCGAGGTGCTCTACTGGGCTGTCCCAGT
GGCAGCGGGAGTCGGCCTTGTGGTGGGCATCATCGGGACAGTGGTCTGGAGGTGCAGGAGAGGTGGCC
GGAGGGAAGATCCTCCAATGAGCCTGCGCACTGTGGCCCTCTAGGCCCGGGG

NOV65b, CG57399-01

| | | | |
|---|---|---|---|
| Protein Sequence | SEQ ID NO: 980 | 1419 aa | MW at 158435.1kD |

MTWDTALWTSVFLIGLLPTLGFANCILQTSGKMCTLRGRYPQPPQPPLCLSPLVHQLRPADIKVVAAL
GNDETFQESGAGQLSEPDPRQWSWPQACLPGVKKEMQDVVGERTPSRRRSLRRREALVPAAGKESLCR
QDIFISLLEIIKHFPPSPQDINLEKDWKLVTLFIGVNDLCHYCPLVQGPVIDLGGMDTLHSLQLPRAF
VNVVEVMELASLYQGQGGKCAMLAAQEAWNSLLASSRYSEQESFTVVFQPFFYETTPSDPRLQDSTTL
AWHLWNRMMEPAGEKDEPLSVKHGRPMKCPSQESPYLFSYRNSNYLTRLQKPQDKLVREGAEIRCPDK
DPSDTVPTSVHRLKPADINVIGALGDSLTAGNGAGSTPGNVLDVLTQYRGLSWSVGGDENIGTVTTLA
DILREFNPSLKGFSVGTGKETSPNAFLNQAVAGGRAEQARRLVDLMKNDTRIHFQEDWKIITLFIGGN
DLCDFCNDLVHYSPQNFTDNIGKALDILHAESQVPRAFVNLVTVLEIVNLRELYQEKKVYCPRMILRS
LCPCVLKFDDNSTELATLIEFNKKFQEKTHQLIESGRYDTREDFTVVVQPFFENVDMPKTQEGLPDNS
FFAPDCFHFSSKSHSRAASALWNNNLEPVGQKTTRHKFENKINITCPSQVQPFLRTYKNSMQGHGTWL
PCRDRAPSALHPTSVHALRPADIQVVAALGDSLTAGNGIGSKPDDLPDVTTQYRGLSYSAGGDGSLEN
VTTLPSSILREFNRNLTGYAVGTGDANDTNAFLNQAVPGAKARDLMSQVQTLMQKMKDDHRVNFHEDW
KVITVLIGGSDLCDYCTDSNLYSAANFVHHLRNALDVLHREVPRVLVNLVDFLNPTIMRQVFLGNPDK
CPVQQASVLCNCVLTLRENSQELARLEAFSRAYQSSMRELVGSGRYDTQEDFSVVLQPFFQNIQLPVL
QDGLPDTSFFAPDCIHPNQKFHSQLARALWTNMLEPLGSKTETLDLRAEMPITCPTQNEPFLRTPRNS
NYTYPIKPAIENWGSDFLCTEWKASNSVPTSVHQLRPADIKVVAALGDSLTTAVGARPNNSSDLPTSW
RGLSWSIGGDGNLETHTTLPSILKKFNPYLLGFSTSTWEGTAGLNVAAEGARARRDMPAQAWDLVERM
KNSPIHFQEDWKIITLFIGGNDLCDFCNDLVGEYVQHIQQALDILSEELPRAFVNVVEVMELASLYQG
QGGKCAMLAAQNNCTCLRHSQSSLEKQELKKVNWNLQHGISSFSYWHQYTQREDFAVVVQPFFQNTLT
PLNRGDTDLTFFSEDCFHFSDRGHAEMAIALWNNMLEPVGRKTTSNNFTHSRAKLKCPSPVSPYLYTL
RNSRLLPDQAEEAPEVLYWAVPVAAGVGLVVGIIGTVVWRCRRGGRREDPPMSLRTVAL

| | | |
|---|---|---|
| NOV65c, CG57399-02 | SEQ ID NO: 981 | 1624 bp |

| | | |
|---|---|---|
| DNA Sequence | ORF Start: ATG at 311 | ORF Stop: TGA at 1241 |

GCCGGCTGACATCAATGTAATTGGAGCCCTGGGTGACTCTCTCACGGCAGGCAATGGGGCCGGGTCCA
CACCTGGGAACGTCTTGGACGTCTTGACTCAGTACCGAGGCCTGTCCTGGAGCGTCGGCGGAGATGAG
AACATCGGCACCGTTACCACCCTGGCGAACATCCTCCGGGAATTCAACCCTTCCCTGAAGGGCTTCTC
TGTTGGCACTGGGAAAGAAACCAGTCCTAATGCCTTCTTAAACCAGGCTGTGGCAGGAGGCCGAGCTG
AGGATCTACCTGTCCAGGCCAGGAGGCTGGTGGACCTGATGAAGAATGACACGAGGATACACTTTCAG
GAAGACTGGAAGATAATAACCCTGTTTATAGGCGGCAATGACCTCTGTGATTTcTGCAATGATCTGGT
CCACTATTCTCCCCAGAACTTCACAGACAACATTGGAAAGGCCCTGGACATCCTCCATGCTGAGGTTC
CTCGGGCATTTGTGAACCTGGTGACGGTGCTTGAGATCGTCAACCTGAGGGAGCTGTACCAGGAGAAA
AAAGTCTACTGCCCAAGGATGATCCTCAGGTCTCTGTGTCCCTGTGTCCTGAAGTTTGATGATAACTC
AACAGAACTTGCTACCCTCATCGAATTCAACAAGAAGTTTCAGGAGAAGACCCACCAACTGATTGAGA
GTGGGCGATATGACACAAGGGAAGATTTTACTGTGGTTGTGCAGCCGTTCTTTGAAAACGTGGACATG
CCAAAGACCTCGGAAGGATTGCCTGACAACTCTTTCTTCGCTCCTGACTGTTTCCACTTCAGCAGCAA
GTCTCACTCCCGAGCAGCCAGTGCTCTCTGGAACAATATGCTGGAGCCTGTTGGCCAGAAGACGACTC
GTCATAAGTTTGAAAACAAGATCAATATCACATGTCCGAACCAGGTCCAGCCGTTTCTGAGGACCTAC
AAGAACAGCATGCAGGGTCATGGGACCTGGCTGCCATGCAGGGACAGAGCCCCTTCTGCCTTGCACCC
TACCTCAGTGCATGCCCTGAGACCTGCAGACATCCAAGTTGTGGCTGCTCTGGGGGATTCTCTGACCG
CTGGCAATGGAATTGGCTCCAAACCAGACGACCTCCCCGATGTCACCACACAGTATCGGGGACTGTCA
TACAGAGAAAGTAAACCAGGGTTCTTATCAGACTCCTGGGTCAGCAAATCCAACAGGAAATGCACCAG
AAAAGCACCAAATCCCTGAATCTTCACCTCCCCGCTTGCATGTATACGTGTACACGTGGTGTTCCTAC
GTCTCTGTTTACTGTCTTTATGTGTTTATTCATGTTGTCTTGTAGTCACACAGCTGCCTTTACATATA
TGTACACATCTGCACAGAAAACCTCTGAAACCCATCGCACACTTCGAGAGGCCATAACCAAGACACAA
TCACAATCAGCCATGTCTTGAAAGATTAGCAATTCGACAAGAGGAAAGGGTGAGAAAGGGCATCCCGA
ACACGGAAGTGGAGAAGCTCAGGGTGTGTCAGGCGAGCGGTTGCGTGTAGATATTCTCAAGTTTCTTT
CTCTCCTAATAAAGTTCTCATTCCTGTAGGCTTCAAAGTAAGTGGCGAGTAGCTCAGAAT

NOV65c, CG57399-02

| | | | |
|---|---|---|---|
| Protein Sequence | SEQ ID NO: 982 | 310 aa | MW at 35240.6kD |

MKNDTRIHFQEDWKIITLFIGGNDLCDFCNDLVHYSPQNFTDNIGKALDILHAEVPRAFVNLVTVLEI
VNLRELYQEKKVYCPRMILRSLCPCVLKFDDNSTELATLIEFNKKFQEKTHQLIESGRYDTREDFTVV
VQPFFENVDMPKTSEGLPDNSFFAPDCFHFSSKSHSRAASALWNNMLEPVGQKTTRHKFEMKINITCP
NQVQPFLRTYKNSMQGHGTWLPCRDRAPSALHPTSVHALRPADIQVVAALGDSLTAGNGIGSKPDDLP
DVTTQYRGLSYRESKPGFLSDSWVSKSNRKCTRKAPNP

| | | |
|---|---|---|
| NOV65d, CG57399-03 | SEQ ID NO: 983 | 4425 bp |

| | | |
|---|---|---|
| DNA Sequence | ORF Start: ATG at 16 | ORF Stop: TAG at 4285 |

CTGGAGCATTCTGGCATGGGGCTGCGGCCAGGCATTTTCCTCCTGGAGCTGCTGCTGCTTCTGGGGCA
AGGTACCCCTCAGATCCATACCTCTCCTAGAAAGAGTACATTGGAAGGGCAGCTATGGCCAGAGACAG
TTCACTCTCTGAAGCCTTCTGATATTAAATTTGTGGCAGCCATTGGCAATCTGGAAATTGTGCCAGAC
CCAGGGACGGGCGATCTGGAGAAGCAAGACGAAAGGCCACAGCAGGTGTGCATGGGAGTGATGACAGT
CCTTTCAGACATCATCAGATATTTCAGTCCTTCTGTTCCAATGCCTGTGTGCCACACTGGAAAGAGAG
TCATACCCCACGATGGTGCTGAGGACTTGTGGATTCAGGCTCAAGAACTGGTGAGAAACATGAAAGAG
AACCAACTTGACTTTCAATTTGACTGGAAGCTCATCAATGTGTTCTTCAGTAATGCAAGCCAGTGTTA
CCTGTGCCCCTCTGCTCAACAGAATGGGCTTGCGGCGGGCGGCGTGGATGAGCTGATGGGGGTGCTGG
ACTACCTGCAGCAGGAGGTGCCCAGAGCATTTGTAAACCTGGTGGACCTCTCTGAGGTTGCAGAGGTC
TCTCGTCAGTATCACGGCACTTGGCTCAGCCCTGCACCAGAGCCCTGTAATTGCTCAGAGGAGACCAC
CCGGCTGGCCAAGGTGGTGATGCAGTGGTCTTATCAGGAAGCCTGGAACAGCCTCCTGGCCTCCAGCA
GGTACAGTGAGCAGGAGTCCTTCACCGTGGTTTTCCAGCCTTTCTTCTATGAGACCACCCCATCTGAC
CCCCGACTCCAGGATTCTACCACGCTGGCCTGGCATCTCTGGAATAGGATGATGGAGCCAGCAGGAGA
GAAAGATGAGCCATTGAGTGTAAAACACGGGAGGCCAATGAAGTGTCCCTCTCAGGAGAGCCCCTATC
TGTTCAGCTACAGAAACAGCAACTACCTGACCAGACTGCAGAAACCCCAAGACAAGCTTGAGGTAAGA
GAAGGAGCGGAAATCAGATGTCCTGACAAAGACCCCTCCGATACGGTTCCCACCTCAGTTCATAGGCT
GAAGCCGGCTGACATCAACGTAATTGGAGCCCTGGGTGACTCTCTCACGGCAGGCAATGGGGCCGGGT
CCACACCTGGGAACGTCTTGGACGTCTTGACTCAGTACCGAGGCCTGTCCTGGAGCGTCGGCGGAGAT
GAGAACATCGGCACCGTTACCACCCTGGCGGACATCCTCCGGGAATTCAACCCTTCCCTGAAGGGCTT
CTCTGTTGGCACTGGGAAAGAAACCAGTCCTAATGCCTTCTTAAACCAGGCTGTGGCAGGAGGCCGAG
CTGAGCAGGCCAGGAGGCTGGTGGACCTGATGAAGAATGACACGAGGATACACTTTCAGGAAGACTGG
AAGATAATAACCCTGTTTATAGGCGGCAATGACCTCTGTGATTTCTGCAATGATCTGGTACACTATTC
TCCCCAGAACTTCACAGACAACATTGGAAAGGCCCTGGACATCCTCCATGCTGAGGTTCCTCGGGCAT
TTGTGAACCTGGTGACGGTGCTTGAGATCGTCAACCTGAGGGAGCTGTACCAGGAGAAAAAAGTCTAC
TGCCCAAGGATGATCCTCAGGTCACTGTGTCCCTGTGTCCTGAAGTTTGATGATAACTCAACAGAACT
TGCTACCCTCATCGAATTCAACAAGAAGTTTCAGGAGAAGACCCACCAACTGATTGAGAGTGGGCGAT
ATGACACAAGGGAAGATTTTACTGTGGTTGTGCAGCCGTTCTTTGAAAACGTGGACATGCCAAAGACC
CAGGAAGGATTGCCTGACAACTCTTTCTTCGCTCCTGACTGTTTCCACTTCAGCAGCAAGTCTCACTC
CCGAGCAGCCAGTGCTCTCTGGAACAATATGCTGGAGCCTGTTGGCCAGAAGACGACTCGTCATAAGT
TTGAAAACAAGATCAATATCACATGTCCGAACCAGGTAGAGTGGCCGTTTCTGAGGACCTACAAGAAC
AGCATGCAGGGTCATGGGACCTGGCTGCCATGCAGGGACAGAGCCCCTTCTGCCTTGCACCCTACCTC
AGTGCATGCCCTGAGACCTGCAGACATCCAAGTTGTGGCTGCTCTGGGGGATTCTCTGACCGCTGGCA
ATGGAATTGGCTCCAAACCAGACGACCTCCCCGATGTCACCACACAGTATCGGGGACTGTCATACAGT
GCAGGAGGGGACGGCTCCCTGGAGAATGTGACCACCTTACCTGATATCCTTCGGGAGTTTAACAGAAA
CCTCACAGGCTACGCCGTGGGCACGGGTGATGCCAATGACACGAATGCATTCCTCAATCAAGCTGTTC
CCGGAGCAAAGGCTAGGGATCTTATGAGCCAAGTCCAAACTCTGATGCAGAAGATGAAAGATGATCAT
AGAGTAAATTTCCATGAAGACTGGAAGGTCATCACAGTGCTGATCGGAGGCAGCGATTTATGTGACTA
CTGCACAGATTCGAATCTGTATTCTGCAGCCAACTTTGTTCACCATCTCCGCAATGCCTTGGACGTCC
TGCATAGAGAGGTGCCCAGAGTCCTGGTCAACCTCGTGGACTTCCTGAACCCCACTATCATGCGGCAG
GTGTTCCTGGGAAACCCAGACAAGTGCCCAGTGCAGCAGGCCAGCGTTTTGTGTAACTGCGTTCTGAC
CCTGCGGGAGAACTCCCAAGAGCTAGCCAGGCTGGAGGCCTTCAGCCGAGCCTACCAGAGCAGCATGC
GCGAGCTGGTGGGGTCAGGCCGCTATGACACGCAGGAGGACTTCTCTGTGGTGCTGCAGCCCTTCTTC
CAGAACATCCAGCTCCCTGTCCTGCAGGATGGGCTCCCAGATACGTCCTTCTTTGCCCCAGACTGCAT
CCACCCAAATCAGAAATTCCACTCCCAGCTGGCCAGAGCCCTTTGGACCAATATGCTTGAACCACTTG
GAAGCAAAACAGAGACCCTGGACCTGAGAGCAGAGATGCCCATCACCTGTCCCACTCAGAATGAGCCC
TTCCTGAGAACCCCTCGGAATAGTAACTACACGTACCCCATCAAGCCAGCCATTGAGAACTGGGGCAG
TGACTTCCTGTGTACAGAGTGGAAGGCTTCCAATAGTGTTCCAACCTCTGTCCACCAGCTCCGACCAG
CAGACATCAAAGTGGTGGCCGCCCTGGGTGACTCTCTGACTGTGGCAGTGGGAGCTCGACCAAACAAC
TCCAGTGACCTACCCACATCTTGGAGGGGACTCTCTTGGAGCATTGGAGGGGATGGGAACTTGGAGAC
TCACACCACACTGCCCGACATTCTGAAGAAGTTCAACCCTTACCTCCTTGGCTTCTCTACCAGCACCT
GGGAGGGGACAGCAGGACTAAATGTGGCAGCGGAAGGGGCCAGAGCTAGGGACATGCCAGCCCAGGCC
TGGGACCTGGTAGAGCGAATGAAAAACAGCCCCCAGGACATCAACCTGGAGAAAGACTGGAAGCTGGT
CACACTCTTCATTGGGGTCAACGACTTGTGTCATTACTGTGAGAATCCGGTAGGCGAATATGTTCAGC
ACATCCAACAGGCCCTGGACATCCTCTCTGAGGAGCTCCCAAGGGCTTTCGTCAACGTGGTGGAGGTC
ATGGAGCTGGCTAGCCTGTACCAGGGCCAAGGCGGGAAATGTGCCATGCTGGCAGCTCAGAACAACTG
CACTTGCCTCAGACACTCGCAAAGCTCCCTGGAGAAGCAAGAACTGAAGAAAGTGAACTGGAACCTCC
AGCATGGCATCTCCAGTTTCTCCTACTGGCACCAATACACACAGCGTGAGGACTTTGCGGTTGTGGTG
CAGCCTTTCTTCCAAAACACACTCACCCCACTGAACAGAGGGGACACTGACCTCACCTTCTTCTCCGA
GGACTGTTTTCACTTCTCAGACCGCGGGCATGCCGAGATGGCCATCGCACTCTGGAACAACATGCTGG
AACCAGTGGGCCGCAAGACTACCTCCAACAACTTCACCCACAGCCGAGCCAAACTCAAGTGCCCCTCT
CCTGAGAGCCCTTACCTCTACACCCTGCGGAACAGCCGATTGCTCCCAGACCAGGCTGAAGAAGCCCC
CGAGGTGCTCTACTGGGCTGTCCCAGTGGCAGCGGGAGTCGGCCTTGTGGTGGGCATCATCGGGACAG
TGGTCTGGAGGTGCAGGAGAGGTGGCCGGAGGGAAGATCCTCCAATGAGCCTGCGCACTGTGGCCCTC
TAGGCCCGGGGGTGGGTCCTCACCCTAAACTCCCTATAGCCACTCTCTTCACCGCCCTCTGCCCCAGC
CACTCCCGGCCACCAGGACATGCTTCAATGCCTGGTGCCATAGGAAGCCCAGGGGACAGTCACAACTT
CTTGG

NOV65d, CG57399-03

| | | | |
|---|---|---|---|
| Protein Sequence | SEQ ID NO: 984 | 1423 aa | MW at 159352.7kD |

MGLRPGIFLLELLLLLGQGTPQIHTSPRKSTLEGQLWPETVHSLKPSDIKFVAAIGNLEIVPDPGTGD
LEKQDERPQQVCMGVMTVLSDIIRYFSPSVPMPVCHTGKRVIPHDGAEDLWIQAQELVRNMKENQLDF
QFDWKLINVFFSNASQCYLCPSAQQNGLAAGGVDELNGVLDYLQQEVPRAFVNLVDLSEVAEVSRQYH
GTWLSPAPEPCNCSEETTRLAKVVMQWSYQEAWNSLLASSRYSEQESFTVVFQPFFYETTPSDPRLQD
STTLAWHLWNRMMEPAGEKDEPLSVKHGRPMKCPSQESPYLFSYRNSNYLTRLQKPQDKLEVREGAEI
RCPDKDPSDTVPTSVHRLKPADINVIGALGDSLTAGNGAGSTPGNVLDVLTQYRGLSWSVGGDENIGT
VTTLADILREFNPSLKGFSVGTGKETSPNAFLNQAVAGGRAEQARRLVDLMKNDTRIHFQEDWKIITL
FIGGNDLCDFCNDLVHYSPQNFTDNIGKALDILHAEVPRAFVNLVTVLEIVNLRELYQEKKVYCPRMI
LRSLCPCVLKFDDNSTELATLIEFNKKFQEKTEQLIESGRYDTREDFTVVVQPFFENVDMPKTQEGLP
DNSFFAPDCFHFSSKSHSRAASALWNNMLEPVGQKTTRHKFENKINITCPNQVEWPFLRTYKNSMQGH
GTWLPCRDRAPSALHPTSVHALRPADIQVVAALGDSLTAGNGIGSKPDDLPDVTTQYRGLSYSAGGDG
SLENVTTLPDILREFNRNLTGYAVGTGDANDTNAFLNQAVPGAKARDLMSQVQTLMQKMKDDHRVNFH
EDWKVITVLIGGSDLCDYCTDSNLYSAANFVHHLRNALDVLHREVPRVLVNLVDFLNPTIMRQVFLGN
PDKCPVQQASVLCNCVLTLRENSQELARLEAFSRAYQSSMRELVGSGRYDTQEDFSVVLQPFFQNIQL
PVLQDGLPDTSFFAPDCIHPNQKFHSQLARALWTNNLEPLGSKTETLDLRAEMPITCPTQMEPFLRTP
RNSNYTYPIKPAIENWGSDFLCTEWKASNSVPTSVHQLRPADIKVVAALGDSLTVAVGARPNNSSDLP
TSWRGLSWSIGGDGNLETHTTLPDILKKFNPYLLGFSTSTWEGTAGLNVAAEGARARDMPAQAWDLVE
RMKNSPQDINLEKDWKLVTLFIGVNDLCHYCENPVGEYVQHIQQALDILSEELPRAFVNVVEVMELAS
LYQGQGGKCANLAAQNNCTCLRHSQSSLEKQELKKVNWNLQHGISSFSYWHQYTQREDFAVVVQPFFQ
NTLTPLNRGDTDLTFFSEDCFHFSDRGHAEMAIALWNNMLEPVGRKTTSNNFTHSRAKLKCPSPESPY
LYTLRNSRLLPDQAEEAPEVLYWAVPVAAGVGLVVGIIGTVVWRCRRGGRREDPPMSLRTVAL

A ClustalW comparison of the above protein sequences yields the following sequence alignment shown in Table 65B.

TABLE 65B
Comparison of the NOV65 protein sequences.

| | |
|---|---|
| NOV65a | ------------------------------------------------------------ |
| NOV65b | MTWDTALWTSVFLIGLLPTLGFANCILQTSGKMCTLRGRYPQPPQPPLCLSPLVHQLRPA |
| NOV65c | ------------------------------------------------------------ |
| NOV65d | ----MGLRPGIFLLELLLLLGQGTPQIHTSPRKSTLEGQLWP---------ETVHSLKPS |
| NOV65a | ------------------------------------------------------------ |
| NOV65b | DIKVVAALGNDETFQESGAGQLSEPDPRQWSWPQACLPGVKKEMQDVVGERTPSRRRSLR |
| NOV65c | ------------------------------------------------------------ |
| NOV65d | DIKFVAAIGNLEIVPDPGTGDLEKQDER----PQQVCMGVMTVLSDIIRYFSPSVPMPVC |
| NOV65a | ------------------------------------------------------------ |
| NOV65b | RR-EALVPAAGKESLCRQDIFISLLEIIKHFPPSPQDINLEKDWKLVTLFIGVNDLCHYC |
| NOV65c | ------------------------------------------------------------ |
| NOV65d | HTGKRVIPHDG-----AEDLWIQAQELVRNMKEN--QLDFQFDWKLINVFFSNASQCYLC |
| NOV65a | ------------------------------------------------------------ |
| NOV65b | PLVQGPVIDLGGMDTLH-----S-LQLPRAFVNVVEVMELASLYQGQGG----------- |
| NOV65c | ------------------------------------------------------------ |
| NOV65d | PSAQQNGLAAGGVDELMGVLDYLQQEVPRAFVNLVDLSEVAEVSRQYHGTWLSPAPEPCN |
| NOV65a | ------------------------------------------------------------ |
| NOV65b | -------K---CAMLAAQEAWNSLLASSRYSEQESFTVVFQPFFYETTPSDPRLQDSTTL |
| NOV65c | ------------------------------------------------------------ |
| NOV65d | CSEETTRLAKVVMQWSYQEAWNSLLASSRYSEQESFTVVFQPFFYETTPSDPRLQDSTTL |
| NOV65a | ------------------------------------------------------------ |
| NOV65b | AWHLWNRMMEPAGEKDEPLSVKHGRPMKCPSQESPYLFSYRNSNYLTRLQKPQDK-LVRE |
| NOV65c | ------------------------------------------------------------ |
| NOV65d | AWHLWNRMMEPAGEKDEPLSVKHGRPMKCPSQESPYLFSYRNSNYLTRLQKPQDKLEVRE |
| NOV65a | ------------------------------------------------------------ |
| NOV65b | GAEIRCPDKDPSDTVPTSVHRLKPADINVIGALGDSLTAGNGAGSTPGNVLDVLTQYRGL |
| NOV65c | ------------------------------------------------------------ |
| NOV65d | GAEIRCPDKDPSDTVPTSVHRLKPADINVIGALGDSLTAGNGAGSTPGNVLDVLTQYRGL |
| NOV65a | ------------------------------------------------------------ |
| NOV65b | SWSVGGDENIGTVTTLADILREFNPSLKGFSVGTGKETSPNAFLNQAVAGGRAEQARRLV |
| NOV65c | ------------------------------------------------------------ |
| NOV65d | SWSVGGDENIGTVTTLADILREFNPSLKGFSVGTGKETSPNAFLNQAVAGGRAEQARRLV |
| NOV65a | --MKNDTRIHFQEDWKIITLFIGGNDLCDFCNDLVHYSPQNFTDNIGKALDILHAE--VP |
| NOV65b | DLMKNDTRIHFQEDWKIITLFIGGNDLCDFCNDLVHYSPQNFTDNIGKALDILHAESQVP |
| NOV65c | --MKNDTRIHFQEDWKIITLFIGGNDLCDFCNDLVHYSPQNFTDNIGKALDILHAE--VP |
| NOV65d | DLMKNDTRIHFQEDWKIITLFIGGNDLCDFCNDLVHYSPQNFTDNIGKALDILHAE--VP |
| NOV65a | RAFVNLVTVLEIVNLRELYQEKKVYCPRMILRSLCPCVLKFDDNSTELATLIEFNKKFQE |
| NOV65b | RAFVNLVTVLEIVNLRELYQEKKVYCPRMILRSLCPCVLKFDDNSTELATLIEFNKKFQE |
| NOV65c | RAFVNLVTVLEIVNLRELYQEKKVYCPRMILRSLCPCVLKFDDNSTELATLIEFNKKFQE |
| NOV65d | RAFVNLVTVLEIVNLRELYQEKKVYCPRNILRSLCPCVLKFDDNSTELATLIEFNKKFQE |
| NOV65a | KTHQLIESGRYDTREDFTVVVRPFFENVDMPKTSEGLPDNSFFAPDCFHFSSKSHSRAAS |
| NOV65b | KTHQLIESGRYDTREDFTVVVQPFFENVDMPKTQEGLPDNSFFAPDCFHFSSKSHSRAAS |
| NOV65c | KTHQLIESGRYDTREDFTVVVQPFFENVDMPKTSEGLPDNSFFAPDCFHFSSKSHSRAAS |
| NOV65d | KTHQLIESGRYDTREDFTVVVQPFFENVDMPKTQEGLPDNSFFAPDCFHFSSKSHSRAAS |
| NOV65a | ALWNNMLEPVGQKTTRHKFENKINITCPNQVQP-FLRTYKNSMQGHGTWLPCRDRAPSAL |
| NOV65b | ALWNNMLEPVGQKTTRHKFENKINITCPSQVQP-FLRTYKNSMQGHGTWLPCRDRAPSAL |
| NOV65c | ALWNNMLEPVGQKTTRHKFENKINITCPNQVQP-FLRTYKNSMQGHGTWLPCRDRAPSAL |
| NOV65d | ALWNNMLEPVGQKTTRHKFENKINITCPNQVEWPFLRTYKNSMQGHGTWLPCRDRAPSAL |
| NOV65a | HPTSVHALRPADIQVVAALGDSLTAGNGIGSKPDDLPDVTTQYRGLSYRESKPG------ |
| NOV65b | HPTSVHALRPADIQVVAALGDSLTAGNGIGSKPDDLPDVTTQYRGLSYSAGGDGSLENVT |
| NOV65c | HPTSVHALRPADIQVVAALGDSLTAGNGIGSKPDDLPDVTTQYRGLSYRESKPG------ |
| NOV65d | HPTSVHALRPADIQVVAALGDSLTAGNGIGSKPDDLPDVTTQYRGLSYSAGGDGSLENV- |
| NOV65a | FLSDSWVSKSNRKCTRKAPNP--------------------------------------- |
| NOV65b | TLPSSILREFNRNLTGYAVGTGDANDTNAFLNQAVPGAKARDLMSQVQTLMQKMKDDHRV |
| NOV65c | FLSDSWVSKSNRKCTRKAPNP--------------------------------------- |
| NOV65d | TTLPDILREFNRLTGYAVGTGDANDTNAFLNQAVPGAKARDLMSQVQTLMQKMKDDHRV |
| NOV65a | ------------------------------------------------------------ |
| NOV65b | NFHEDWKVITVLIGGSDLCDYCTDSNLYSAANFVHHLRNALDVLHREVPRVLVNLVDFLN |
| NOV65c | ------------------------------------------------------------ |
| NOV65d | NFHEDWKVITVLIGGSDLCDYCTDSNLYSAANFVHHLRNALDVLHREVPRVLVNLVDFLN |
| NOV65a | ------------------------------------------------------------ |
| NOV65b | PTIMRQVFLGNPDKCPVQQASVLCNCVLTLRENSQELARLEAFSRAYQSSMRELVGSGRY |
| NOV65c | ------------------------------------------------------------ |
| NOV65d | PTIMRQVFLGNPDKCPVQQASVLCNCVLTLRENSQELARLEAFSRAYQSSMRELVGSGRY |
| NOV65a | ------------------------------------------------------------ |
| NOV65b | DTQEDFSVVLQPFFQNIQLPVLQDGLPDTSFFAPDCIHPNQKFHSQLARALWTNMLEPLG |
| NOV65c | ------------------------------------------------------------ |
| NOV65d | DTQEDFSVVLQPFFQNIQLPVLQDGLPDTSFFAPDCIHPNQKFHSQLARALWTNMLEPLG |
| NOV65a | ------------------------------------------------------------ |
| NOV65b | SKTETLDLRAEMPITCPTQNEPFLRTPRNSNYTYPIKPAIENWGSDFLCTEWKASNSVPT |
| NOV65c | ------------------------------------------------------------ |
| NOV65d | SKTETLDLRAEMPITCPTQNEPFLRTPRNSNYTYPIKPAIENWGSDFLCTEWKASNSVPT |
| NOV65a | ------------------------------------------------------------ |
| NOV65b | SVHQLRPADIKVVAALGDSLTTAVGARPNNSSDLPTSWRGLSWSIGGDGNLETHTTLPSI |
| NOV65c | ------------------------------------------------------------ |
| NOV65d | SVHQLRPADIKVVAALGDSLTVAVGARPNNSSDLPTSWRGLSWSIGGDGNLETHTTLPDI |
| NOV65a | ------------------------------------------------------------ |
| NOV65b | LKKFNPYLLGFSTSTWEGTAGLNVAAEGARARRDMPAQAWDLVERMKNSP--IHFQEDWK |
| NOV65c | ------------------------------------------------------------ |
| NOV65d | LKKFNPYLLGFSTSTWEGTAGLNVAAEGARAR-DMPAQAWDLVERMKNSPQDINLEKDWK |
| NOV65a | ------------------------------------------------------------ |
| NOV65b | IITLFIGGNDLCDFCNDLVGEYVQHIQQALDILSEELPRAFVNVVEVMELASLYQGQGGK |
| NOV65c | ------------------------------------------------------------ |
| NOV65d | LVTLFIGVNDLCHYCENPVGEYVQHIQQALDILSEELPRAFVNVVEVMELASLYQGQGGK |
| NOV65a | ------------------------------------------------------------ |
| NOV65b | CAMLAAQNNCTCLRHSQSSLEKQELKKVNWNLQHGISSFSYWHQYTQREDFAVVVQPFFQ |
| NOV65c | ------------------------------------------------------------ |
| NOV65d | CAMLAAQNNCTCLRHSQSSLEKQELKKVNWNLQHGISSFSYWHQYTQREDFAVVVQPFFQ |
| NOV65a | ------------------------------------------------------------ |
| NOV65b | NTLTPLNRGDTDLTFFSEDCFHFSDRGHAEMAIALWNNMLEPVGRKTTSNNFTHSRAKLK |
| NOV65c | ------------------------------------------------------------ |
| NOV65d | NTLTPLNRGDTDLTFFSEDCFHFSDRGHAEMAIALWNNMLEPVGRKTTSNNFTHSRAKLK |
| NOV65a | ------------------------------------------------------------ |
| NOV65b | CPSPVSPYLYTLRNSRLLPDQAEEAPEVLYWAVPVAAGVGLVVGIIGTVVWRCRRGGRRE |
| NOV65c | ------------------------------------------------------------ |
| NOV65d | CPSPESPYLYTLRNSRLLPDQAEEAPEVLYWAVPVAAGVGLVVGIIGTVVWRCRRGGRRE |
| NOV65a | ----------- |
| NOV65b | DPPMSLRTVAL |
| NOV65c | ----------- |
| NOV65d | DPPMSLRTVAL |
| NOV65a | (SEQ ID NO: 978) |
| NOV65b | (SEQ ID NO: 980) |
| NOV65c | (SEQ ID NO: 982) |
| NOV65d | (SEQ ID NO: 984) |

Further analysis of the NOV65a protein yielded the following properties shown in Table 65C.

TABLE 65C
Protein Sequence Properties NOV65a

| | |
|---|---|
| SignalP analysis: | No Known Signal Sequence Indicated |
| PSORT II analysis: | |

| PSG: | a new signal peptide prediction method |
|---|---|
| | N-region: length 11; pos.chg 2; neg.chg 2 |
| | H-region: length 0; peak value −1.15 |
| | PSG score: −5.55 |
| GvH: | von Heijne's method for signal seq. recognition |
| | GvH score (threshold: −2.1): −10.77 |
| | possible cleavage site: between 28 and 29 |

>>> Seems to have no N-terminal signal peptide
ALOM: Klein et al's method for TM region allocation

Init position for calculation: 1
Tentative number of TMS(s) for the threshold 0.5: 0
number of TMS(s) . . . fixed
PERIPHERAL Likelihood = 1.43 (at 55)
ALOM score: 1.43 (number of TMSs: 0)

MITDISC: discrimination of mitochondrial targeting seq

| | | | |
|---|---|---|---|
| R content: | 1 | Hyd Moment (75): | 3.74 |
| Hyd Moment (95): | 6.40 | G content: | 0 |
| D/E content: | 2 | S/T content: | 1 |

Score: −6.13

Gavel: prediction of cleavage sites for mitochondrial preseq

cleavage site motif not found

NUCDISC: discrimination of nuclear localization signals

pat4: none
pat7: none
bipartite: none
content of basic residues: 12.3%
NLS Score: −0.47

KDEL: ER retention motif in the C-terminus: none
ER Membrane Retention Signals:

KKXX-like motif in the C-terminus: KAPN

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | Prediction: nuclear |
| | Reliability: 70.6 |
| COIL: | Lupas's algorithm to detect coiled-coil regions |
| | total: 0 residues |

--------------------------
Final Results (k = 9/23):

```
87.0%: nuclear
  8.7%: mitochondrial
  4.3%: peroxisomal
```

>> prediction for CG57399-04 is nuc (k = 23)

A search of the NOV65a protein against the Geneseq database, a proprietary database that contains sequences published in patents and patent publication, yielded several homologous proteins shown in Table 65D.

TABLE 65D
Geneseq Results for NOV65a

| | | NOV65a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB09556 | Human lipase NHL (Val 1318 | 1 . . . 293 | 284/293 (96%) | e-170 |
| | variant) - Homo sapiens, 1458 aa. | 491 . . . 783 | 286/293 (96%) | |
| | [WO200259328-A1, 01-Aug.- | | | |
| | 2002] | | | |
| ABB09555 | Human lipase NHL (Ala 1318 | 1 . . . 293 | 284/293 (96%) | e-170 |
| | variant) - Homo sapiens, 1458 aa. | 491 . . . 783 | 286/293 (96%) | |
| | [WO200259328-A1, 01-Aug.- | | | |
| | 2002] | | | |
| AAE22860 | Human phospholipase-like | 1 . . . 293 | 273/298 (91%) | e-160 |
| | enzyme - Homo sapiens, 1216 aa. | 247 . . . 544 | 279/298 (93%) | |
| | [WO200231 161-A2, 18-Apr.- | | | |
| | 2002] | | | |
| AAW30751 | Rat phospholipase-Bllipase - | 1 . . . 293 | 203/293 (69%) | e-118 |
| | Rattus rattus, 1450 aa. | 495 . . . 787 | 236/293 (80%) | |
| | [JP09248190-A, 22-Sep.-1997] | | | |
| ABP53556 | Human phospholipase protein | 1 . . . 217 | 92/218 (42%) | 8e-44 |
| | SEQ ID NO:2 - Homo sapiens, | 203 . . . 416 | 130/218 (59%) | |
| | 472 aa. [WO200262977-A2, 15- | | | |
| | Aug.-2002] | | | |

In a BLAST search of public sequence databases, the NOV65a protein was found to have homology to the proteins shown in the BLASTP data in Table 65E.

TABLE 65E
Public BLASTP Results for NOV65a

| | | NOV65a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96DP9 | Hypothetical protein FLJ30866 - | 1 . . . 259 | 258/259 (99%) | e-154 |
| | Homo sapiens (Human), 270 aa. | 1 . . . 259 | 259/259 (99%) | |
| Q05017 | Phospholipase ADRAB-B | 1 . . . 293 | 238/293 (81%) | e-140 |
| | precursor (EC 3.1.-.-)- | 491 . . . 783 | 260/293 (88%) | |
| | Oryctolagus cuniculus (Rabbit), | | | |
| | 1458 aa. | | | |
| 070320 | Phospholipase B - Cavia | 1 . . . 291 | 221/291 (75%) | e-131 |
| | porcellus (Guinea pig), 1463 aa. | 490 . . . 780 | 254/291 (86%) | |
| 054728 | Phospholipase B - Rattus | 1 . . . 293 | 203/293 (69%) | e-117 |
| | norvegicus (Rat), 1450 aa. | 495 . . . 787 | 236/293 (80%) | |
| Q8IUP7 | Similar to phospholipase B - | 1 . . . 217 | 92/218 (42%) | 2e-43 |
| | Homo sapiens (Human), 423 aa. | 154 . . . 367 | 130/218 (59%) | |

PFam analysis indicates that the NOV65a protein contains the domains shown in the Table 65F.

TABLE 65F
Domain Analysis of NOV65a

| | NOV65a | Identities/ | |
|---|---|---|---|
| | Match | Similarities | |
| Pfam Domain | Region | for the Matched Region | Expect Value |
| Lipase_GDSL | 1 . . . 178 | 39/370 (11%) | 1.3e-10 |
| | | 141/370 (38%) | |

### Example 66

The NOV66 clone was analyzed, and the nucleotide and encoded polypeptide sequences are shown in Table 66A.

TABLE 66A
NOV66 Sequence Analysis

| | | |
|---|---|---|
| NOV66a, CG57562-02 | SEQ ID NO: 985 | 3517 bp |

| | | |
|---|---|---|
| DNA Sequence | ORF Start: ATG at 5 | ORF Stop: TGA at 3506 |

AAAGATGGCGGCAGCGGCGGCGGTGGGCAACGCGGTGCCCTGCGGGGCCCGGCCTTGCGGGGTCCGGC
CTGACGGGCAGCCCAAGCCCGGGCCGCAGCCGCGCGCGCTCCTTGCCGCCGGGCCGGCGCTCATAGCG
AACGGTGACGAGCTGGTGGCTGCCGTGTGGCCGTACCGGCGGTTGGCGCTGTTGCGGCGCCTCACGGT
GCTGCCATTCGCCGGGCTGCTTTACCCGGCCTGGTTGGGTGCCGCAGCCGCTGGCTGCTGGGGCTGGG
GCAGCAGTTGGGTGCAGATCCCCGAAGCTGCGCTGCTCGTGCTTGCCACCATCTGCCTCGCGCACGCG
CTCACTGTCCTCTCGGGGCATTGGTCTGTGCACGCGCATTGCGCGCTCACCTGCACCCCGGAGTACGA
CCCCAGCAAAGCGACCTTTGTGAAGGTGGTGCCAACCCCCAACAATGGCTCCACGGAGCTCGTGGCCC
TGCACCGCAATGAGGGCGAAGACGGGCTTGAGGTGCTGTCCTTCGAATTCCAGAAGATCAAGTATTCC
TACGATGCCCTGGAGAAGAAGCAGTTTCTCCCCGTGGCCTTTCCTGTGGGAAACGCCTTCTCATACTA
TCAGAGCAACAGAGGCTTCCAGGAAGACTCAGAGATCCGAGCAGCTGAGAAGAAATTTGGGAGCAACA
AGGCCGAGATGGTGGTGCCTGACTTCTCGGAGCTTTTCAAGGAGAGAGCCACAGCCCCCTTCTTTGTA
TTTCAGGTGTTCTGTGTGGGGCTCTGGTGCCTGGATGAGTACTGGTACTACAGCGTCTTTACGCTATC
CATGCTGGTGGCGTTCGAGGCCTCGCTGGTGCAGCAGCAGATGCGGAACATGTCGGAGATCCGGAAGA
TGGGCAACAAGCCCCACATGATCCAGGTCTACCGAAGCCGCAAGTGGAGGCCCATTGCCAGTGATGAG
ATCGTACCAGGGGACATCGTCTCCATCGGCCGCTCCCCACAGGAGAACCTGGTGCCATGTGACGTGCT
TCTGCTGCGAGGCCGCTGCATCGTAGACGAGGCCATGCTCACGGGGGAGTCCGTGCCACAGATGAAGG
AGCCCATCGAAGACCTCAGCCCAGACCGGGTGCTGGACCTCCAGGCTGATTCCCGGCTGCACGTCATC
TTCGGGGGCACCAAGGTGGTGCAGCACATCCCCCCACAGAAAGCCACCACGGGCCTGAAGCCGGTTGA
CAGCGGGTGCGTGGCCTACGTCCTGCGGACCGGATTCAACACATCCCAGGGCAAGCTGCTGCGCACCA
TCCTCTTCGGGGTCAAGAGGGTGACTGCGAACAACCTGGAGACCTTCATCTTCATCCTCTTCCTCCTG
GTGTTTGCCATCGCTGCAGCTGCCTATGTATGGATTGAAGGTACCAAGGACCCCAGCCGGAACCGCTA
CAAGCTGTTTCTGGAGTGCACCCTGATCCTCACCTCGGTCGTGCCTCCTGAGCTGCCCATCGAGCTGT
CCCTGGCCGTCAACACCTCCCTCATCGCCCTGGCCAAGCTCTACATGTACTGCACAGAGCCCTTCCGG
ATCCCCTTTGCTGGCAAGGTCGAGGTGTGCTGCTTTGACAAGACGGGGACGTTGACCAGTGACAGCCT
GGTGGTGCGCGGTGTGGCCGGGCTGAGAGACGGGAAGGAGGTGACCCCAGTGTCCAGCATCCCTGTAG
AAACACACCGGGCCCTGGCCTCGTGCCACTCGCTCATGCAGCTGGACGACGGCACCCTCGTGGGTGAC
CCTCTAGAGAAGGCCATGCTGACGGCCGTGGACTGGACGCTGACCAAAGATGAGAAAGTATTCCCCCG
AAGTATTAAAACTCAGGGGCTGAAAATTCACCAGCGCTTTCATTTTGCCAGTGCCCTGAAGCGAATGT
CCGTGCTTGCCTCGTATGAGAAGCTGGGCTCCACCGACCTTTGTTACATCGCGGCCGTGAAGGGGGCC
CCCGAAACTCTGCACTCCATGGCCCGGGAGGTCAAGCGGGAGGCCCTGGAGTGCAGCCTCAAGTTCGT
CGGCTTCATTGTGGTCTCCTGCCCGCTCAAGGCTGACTCGAAGGCCGTGATCCGGGAGATCCAGAATG
CGTCCCACCGGGTGTTCATGATCACGGGAGACAACCCGCTCACTGCATGCCACGTGGCCCAGGAGCTG
CACTTCATTGAAAAGGCCCACACGCTGATCCTGCAGCCTCCCTCCGAGAAAGGCCGGCAGTGCGAGTG
GCGCTCCATTGACGGCAGCATCGTGCTGCCCCTGGCCCGGGGCTCCCCAAAGGCACTGGCCCTGGAGT
ACGCACTGTGCCTCACAGGCGACGGCTTGGCCCACCTGCAGGCCACCGACCCCCAGCAGCTGCTCCGC
CTCATCCCCCATGTGCAGGTGTTCGCCCGTGTGGCTCCCAAGCAGAAGGAGTTTGTCATCACCAGCCT
GAAGGAGCTGGGCTACGTGACCCTCATGTGTGGGGATGGCACCAACGACGTGGGCGCCCTGAAGCATG
CTGACGTGGGTGTGGCGCTCTTGGCCAATGCCCCTGAGCGGGTTGTCGAGCGGCGACGGCGGCCCCGG
GACAGCCCAACCCTGAGCAACAGTGGCATCAGAGCCACCTCCAGGACAGCCAAGCAGCGGTCGGGGCT
CCCTCCCTCCGAGGAGCAGCCAACCTCCCAGAGGGACCGCCTGAGCCAGGTGCTGCGAGACCTCGAGG
ACGAGAGTACGCCCATTGTGAAACTGGGGGATGCCAGCATCGCAGCACCCTTCACCTCCAAGCTCTCA
TCCATCCAGTGCATCTGCCACGTGATCAAGCAGGGCCGCTGCACGCTGGTGACCACGCTACAGATGTT
CAAGATCCTGGCGCTCAATGCCCTCATCCTGGCCTACAGCCAGAGCGTCCTCTACCTGGAGGGAGTCA
AGTTCAGTGACTTCCAGGCCACCCTACAGGGGCTGCTGCTGGCCGGCTGCTTCCTCTTCATCTCCCGT
TCCAAGCCCCTCAAGACCCTCTCCCGAGAACGGCCCCTGCCCAACATCTTCAACCTGTACACCATCCT
CACCGTCATGCTCCAGTTCTTTGTGCACTTCCTGAGCCTTGTCTACCTGTACCGTGAGGCCCAGGCCC
GGAGCCCCGAGAAGCAGGAGCAGTTCGTGGACTTGTACAAGGAGTTTGAGCCAAGCCTGGTCAACAGC
ACCGTCTACATCATGGCCATGGCCATGCAGATGGCCACCTTCGCCATCAATTACAAAGGCCCGCCCTT
CATGGAGAGCCTGCCCGAGAACAAGCCCCTGGTGTGGAGTCTGGCAGTTTCACTCCTGGCCATCATTG
GCCTGCTCCTCGGCTCCTCGCCCGACTTCAACAGCCAGTTTGGCCTCGTGGACATCCCTGTGGAGTTC
AAGCTGGTCATTGCCCAGGTCCTGCTCCTGGACTTCTGCCTGGCGCTCCTGGCCGACCGCGTCCTGCA
GTTCTTCCTGGGGACCCCGAAGCTGAAAGTGCCTTCCTGAGATGGCAGT

NOV66a, CG57562-02

| | | | |
|---|---|---|---|
| Protein Sequence | SEQ ID NO: 986 | 1167 aa | MW at 128749.4kD |

MAAAAAVGNAVPCGARPCGVRPDGQPKPGPQPRALLAAGPALIANGDELVAAVWPYRRLALLRRLTVL
PFAGLLYPAWLGAAAAGCWGWGSSWVQIPEAALLVLATICLAHALTVLSGHWSVHAHCALTCTPEYDP
SKATFVKVVPTPNNGSTELVALHRNEGEDGLEVLSFEFQKIKYSYDALEKKQFLPVAFPVGNAFSYYQ
SNRGFQEDSEIRAAEKKFGSNKAEMVVPDFSELFKERATAPFFVFQVFCVGLWCLDEYWYYSVFTLSM
LVAFEASLVQQQMRNMSEIRKMGNKPHMIQVYRSRKWRPIASDEIVPGDIVSIGRSPQENLVPCDVLL
LRGRCIVDEAMLTGESVPQMKEPIEDLSPDRVLDLQADSRLHVIFGGTKVVQHIPPQKATTGLKPVDS
GCVAYVLRTGFNTSQGKLLRTILFGVKRVTANNLETFIFILFLLVFAIAAAAYVWIEGTKDPSRNRYK
LFLECTLILTSVVPPELPIELSLAVNTSLIALAKLYMYCTEPFRIPFAGKVEVCCFDKTGTLTSDSLV
VRGVAGLRDGKEVTPVSSIPVETHRALASCHSLMQLDDGTLVGDPLEKAMLTAVDWTLTKDEKVFPRS
IKTQGLKIHQRFHFASALKRNSVLASYEKLGSTDLCYIAAVKGAPETLHSMAREVKREALECSLKFVG
FIVVSCPLKADSKAVIREIQNASHRVFMITGDNPLTACHVAQELHFIEKAHTLILQPPSEKGRQCEWR
SIDGSIVLPLARGSPKALALEYALCLTGDGLAHLQATDPQQLLRLIPHVQVFARVAPKQKEFVITSLK
ELGYVTLMCGDGTNDVGALKHADVGVALLANAPERVVERRRRPRDSPTLSNSGIRATSRTAKQRSGLP
PSEEQPTSQRDRLSQVLRDLEDESTPIVKLGDASIAAPFTSKLSSIQCICHVIKQGRCTLVTTLQMFK
ILALNALILAYSQSVLYLEGVKFSDFQATLQGLLLAGCFLFISRSKPLKTLSRERPLPNIFNLYTILT
VHLQFFVHFLSLVYLYREAQARSPEKQEQFVDLYKEFEPSLVNSTVYIMAMAMQMATFAINYKGPPFM
ESLPENKPLVWSLAVSLLAIIGLLLGSSPDFNSQFGLVDIPVEFKLVIAQVLLLDFCLALLADRVLQF
FLGTPKLKVPS

| | | |
|---|---|---|
| NOV66b, CG57562-01 | SEQ ID NO: 987 | 3904 bp |

| | | |
|---|---|---|
| DNA Sequence | ORF Start: ATG at 68 | ORF Stop: TGA at 3680 |

TTACCGGAAGTAAAACTTCGGAAGTGAGGCGTTCCTCTGCCCGGAAGTGAGCGCGGCGCTAGGAAAGA
TGGCGGCAGCGGCGGCGGTGGGCAACGCGGTGCCCTGCGGGGCCCGGCCTTGCGGGGTCCGGCCTGAC
GGGCAGCCCAAGCCCGGGCGCAGCCGGCGCGCGCTCCTTGCCGCCGGGCCGGCGCTCATAGCGAACGG
TGACGAGCTGGTGGCTGCCGTGTGGCCGTACCGGCGGTTGGCGCTGTTGCGGCGCCTCACGGTGCTGC
CATTCGCCGGGCTGCTTTACCCGGCCTGGTTGGGTGCCGCAGCCGCTGGCTGCTGGGGCTGGGGCAGC
AGTTGGGTGCAGATCCCCGAAGCTGCGCTGCTCGTGCTTGCCACCATCTGCCTCGCGCACGCGCTCAC
TGTCCTCTCGGGGCATTGGTCTGTGCACGCGCATTGCGCGCTCACCTGCACCCCGGAGTACGACCCCA
GCAAAGCGACCTTTGTGAAGGTGGTGCCAACCCCCAACAATGGCTCCACGGAGCTCGTGGCCCTGCAC
CGCAATGAGGGCGAAGACGGGCTTGAGGTGCTGTCCTTCGAATTCCAGAAGATCAAGTATTCCTACGA
TGCCCTGGAGAAGAAGCAGTTTCTCCCCGTGGCCTTTCCTGTGGGAAACGCCTTCTCATACTATCAGA
GCAACAGAGGCTTCCAGGAAGACTCAGAGATCCGAGCAGCTGAGAAGAAATTTGGGAGCAACAAGGCC
GAGATGGTGGTGCCTGACTTCTCGGAGCTTTTCAAGGAGAGAGCCACAGCCCCCTTCTTTGTATTTCA
GGTGTTCTGTGTGGGGCTCTGGTGCCTGGATGAGTACTGGTACTACAGCGTCTTTACGCTATCCATGC
TGGTGGCGTTCGAGGCCTCGCTGGTGCAGCAGCAGATGCGGAACATGTCGGAGATCCGGAAGATGGGC
AACAAGCCCCACATGATCCAGGTCTACCGAAGCCGCAAGTGGAGGCCCATTGCCAGTGATGAGATCGT
ACCAGGGGACATCGTCTCCATCGGCCGCTCCCCACAGGAGAACCTGGTGCCATGTGACGTGCTTCTGC
TGCGAGGCCGCTGCATCGTAGACGAGGCCATGCTCACGGGGGAGTCCGTGCCACAGATGAAGGAGCCC
ATCGAAGACCTCAGCCCAGACCGGGTGCTGGACCTCCAGGCTGATTCCCGGCTGCACGTCATCTTCGG
GGGCACCAAGGTGGTGCAGCACATCCCCCCACAGAAAGCCACCACGGGCCTGAAGCCGGTTGACAGCG
GGTGCGTGGCCTACGTCCTGCGGACCGGATTCAACACATCCCAGGGCAAGCTGCTGCGCACCATCCTC
TTCGGGGTCAAGAGGGTGACTGCGAACAACCTGGAGACCTTCATCTTCATCCTCTTCCTCCTGGTGTT
TGCCATCGCTGCAGCTGCCTATGTATGGATTGAAGGTACCAAGGACCCCAGCCGGAACCGCTACAAGC
TGTTTCTGGAGTGCACCCTGATCCTCACCTCGGTCGTGCCTCCTGAGCTGCCCATCGAGCTGTCCCTG
GCCGTCAACACCTCCCTCATCGCCCTGGCCAAGCTCTACATGTACTGCACAGAGCCCTTCCGGATCCC
CTTTGCTGGCAAGGTCGAGGTGTGCTGCTTTGACAAGACGGGGACGTTGACCAGTGACAGCCTGGTGG
TGCGCGGTGTGGCCGGGCTGAGAGACGGGAAGGAGGTGACCCCAGTGTCCAGCATCCCTGTAGAAACA
CACCGGGCCCTGGCCTCGTGCCACTCGCTCATGCAGCTGGACGACGGCACCCTCGTGGGTGACCCTCT
AGAGAAGGCCATGCTGACGGCCGTGGACTGGACGCTGACCAAAGATGAGAAAGTATTCCCCCGAAGTA
TTAAAACTCAGGGGCTGAAAATTCACCAGCGCTTTCATTTTGCCAGTGCCCTGAAGCGAATGTCCGTG
CTTGCCTCGTATGAGAAGCTGGGCTCCACCGACCTCTGCTACATCGCGGCCGTGAAGGGGGCCCCCGA
AACTCTGCACTCCATGTTCTCCCAGTGCCCGCCCGACTACCACCACATCCACACCGAGATCTCCCGGG
AAGGAGCCCGCGTCCTGGCGCTGGGGTACAAGGAGCTGGGACACCTCACTCACCAGCAGGCCCGGGAG
GTCAAGCGGGAGGCCCTGGAGTGCAGCCTCAAGTTCGTCGGCTTCATTGTGGTCTCCTGCCCGCTCAA
GGCTGACTCCAAGGCCGTGATCCGGGAGATCCAGAATGCGTCCCACCGGGTGGTCATGATCACGGGAG
ACAACCCGCTCACTGCATGCCACGTGGCCCAGGAGCTGCACTTCATTGAAAAGGCCCACACGCTGATC
CTGCAGCCTCCCTCCGAGAAAGGCCGGCAGTGCGAGTGGCGCTCCATTGACGGCAGCATCGTGCTGCC
CCTGGCCCGGGGCTCCCCAAAGGCACTGGCCCTGGAGTACGCACTGTGCCTCACAGGCGACGGCTTGG
CCCACCTGCAGGCCACCGACCCCCAGCAGCTGCTCCGCCTCATCCCCCATGTGCAGGTGTTCGCCCGT
GTGGCTCCCAAGCAGAAGGAGTTTGTCATCACCAGCCTGAAGGAGCTGGGCTACGTGACCCTCATGTG
TGGGGATGGCACCAACGACGTGGGCGCCCTGAAGCATGCTGACGTGGGTGTGGCGCTCTTGGCCAATG
CCCCTGAGCGGGTTGTCGAGCGGCGACGGCGGCCCCGGGACAGCCCAACCCTGAGCAACAGTGGCATC
AGAGCCACCTCCAGGACAGCCAAGCAGCGGTCGGGGCTCCCTCCCTCCGAGGAGCAGCCAACCTCCCA
GAGGGACCGCCTGAGCCAGGTGCTGCGAGACCTCGAGGACGAGAGTACGCCCATTGTGAAACTGGGGG
ATGCCAGCATCGCAGCACCCTTCACCTCCAAGCTCTCATCCATCCAGTGCATCTGCCACGTGATCAAG
CAGGGCCGCTGCACGCTGGTGACCACGCTACAGATGTTCAAGATCCTGGCGCTCAATGCCCTCATCCT
GGCCTACAGCCAGAGCGTCCTCTACCTGGAGGGAGTCAAGTTCAGTGACTTCCAGGCCACCCTACAGG
GGCTGCTGCTGGCCGGCTGCTTCCTCTTCATCTCCCGTTCCAAGCCCCTCAAGACCCTCTCCCGAGAA
CGGCCCCTGCCCAACATCTTCAACCTGTACACCATCCTCACCGTCATGCTCCAGTTCTTTGTGCACTT
CCTGAGCCTTGTCTACCTGTACCGTGAGGCCCAGGCCCGGAGCCCCGAGAAGCAGGAGCAGTTCGTGG
ACTTGTACAAGGAGTTTGAGCCAAGCCTGGTCAACAGCACCGTCTACATCATGGCCATGGCCATGCAG
ATGGCCACCTTCGCCATCAATTACAAAGGCCCGCCCTTCATGGAGAGCCTGCCCGAGAACAAGCCCCT
GGTGTGGAGTCTGGCAGTTTCACTCCTGGCCATCATTGGCCTGCTCCTCGGCTCCTCGCCCGACTTCA
ACAGCCAGTTTGGCCTCGTGGACATCCCTGTGGAGTTCAAGCTGGTCATTGCCCAGGTCCTGCTCCTG
GACTTCTGCCTGGCGCTCCTGGCCGACCGCGTCCTGCAGTTCTTCCTGGGGACCCCGAAGCTGAAAGT
GCCTTCCTGAGATGGCAGTGCTGGTACCCACTGCCCACCCTGGCTGCCGCTGGGCGGGAACCCCAACA
GGGCCCCGGGAGGGAACCCTGCCCCCAACCCCCCACAGCAAGGCTGTACAGTCTCGCCCTTGGAAGAC
TGAGCTGGGACCCCCACAGCCATCCGCTGGCTTGGCCAGCAGAACCAGCCCCAAGCCAGCACCTTTGG
TAAATAAAGCAGCATCTGAGATTTTAAA

NOV66b, CG57562-01

| | | | |
|---|---|---|---|
| Protein Sequence | SEQ ID NO: 988 | 1204 aa | MW at 133030.2kD |

MAAAAAVGNAVPCGARPCGVRPDGQPKPGRSRRALLAAGPALIANGDELVAAVWPYRRLALLRRLTVL
PFAGLLYPAWLGAAAAGCWGWGSSWVQIPEAALLVLATICLAHALTVLSGHWSVHAHCALTCTPEYDP
SKATFVKVVPTPNNGSTELVALHRNEGEDGLEVLSFEFQKIKYSYDALEKKQFLPVAFPVGNAFSYYQ
SNRGFQEDSEIRAAEKKFGSNKAEMVVPDFSELFKERATAPFFVFQVFCVGLWCLDEYWYYSVFTLSM
LVAFEASLVQQQMRNMSEIRKMGNKPHMIQVYRSRKWRPIASDEIVPGDIVSIGRSPQENLVPCDVLL
LRGRCIVDEAMLTGESVPQMKEPIEDLSPDRVLDLQADSRLHVIFGGTKVVQHIPPQKATTGLKPVDS
GCVAYVLRTGFNTSQGKLLRTILFGVKRVTANNLETFIFILFLLVFAIAAAAYVWIEGTKDPSRNRYK
LFLECTLILTSVVPPELPIELSLAVNTSLIALAKLYMYCTEPFRIPFAGKVEVCCFDKTGTLTSDSLV
VRGVAGLRDGKEVTPVSSIPVETHRALASCHSLMQLDDGTLVGDPLEKAMLTAVDWTLTKDEKVFPRS
IKTQGLKIHQRFHFASALKRMSVLASYEKLGSTDLCYIAAVKGAPETLHSMFSQCPPDYHHIHTEISR
EGARVLALGYKELGHLTHQQAREVKREALECSLKFVGFIVVSCPLKADSKAVIREIQNASHRVVMITG
DNPLTACHVAQELHFIEKAHTLILQPPSEKGRQCEWRSIDGSIVLPLARGSPKALALEYALCLTGDGL
AHLQATDPQQLLRLIPHVQVFARVAPKQKEFVITSLKELGYVTLMCGDGTNDVGALKHADVGVALLAN
APERVVERRRRPRDSPTLSNSGIRATSRTAKQRSGLPPSEEQPTSQRDRLSQVLRDLEDESTPIVKLG
DASIAAPFTSKLSSIQCICHVIKQGRCTLVTTLQMFKILALNALILAYSQSVLYLEGVKFSDFQATLQ
GLLLAGCFLFISRSKPLKTLSRERPLPNIFNLYTILTVMLQFFVHFLSLVYLYREAQARSPEKQEQFV
DLYKEFEPSLVNSTVYIMAMAMQMATFAINYKGPPFMESLPENKPLVWSLAVSLLAIIGLLLGSSPDF
NSQFGLVDIPVEFKLVIAQVLLLDFCLALLADRVLQFFLGTPKLKVPS

| | |
|---|---|
| SEQ ID NO: 989 | 3517 bp |

| | | |
|---|---|---|
| NOV66c, SNP13380762 of | ORF Start: ATG at 5 | ORF Stop: TGA at 3506 |

| | | |
|---|---|---|
| CG57562-02, DNA Sequence | SNP Pos: 756 | SNP Change: T to C |

AAAGATGGCGGCAGCGGCGGCGGTGGGCAACGCGGTGCCCTGCGGGGCCCGGCCTTGCGGGGTCCGGC
CTGACGGGCAGCCCAAGCCCGGGCCGCAGCCGCGCGCGCTCCTTGCCGCCGGGCCGGCGCTCATAGCG
AACGGTGACGAGCTGGTGGCTGCCGTGTGGCCGTACCGGCGGTTGGCGCTGTTGCGGCGCCTCACGGT
GCTGCCATTCGCCGGGCTGCTTTACCCGGCCTGGTTGGGTGCCGCAGCCGCTGGCTGCTGGGGCTGGG
GCAGCAGTTGGGTGCAGATCCCCGAAGCTGCGCTGCTCGTGCTTGCCACCATCTGCCTCGCGCACGCG
CTCACTGTCCTCTCGGGGCATTGGTCTGTGCACGCGCATTGCGCGCTCACCTGCACCCCGGAGTACGA
CCCCAGCAAAGCGACCTTTGTGAAGGTGGTGCCAACCCCCAACAATGGCTCCACGGAGCTCGTGGCCC
TGCACCGCAATGAGGGCGAAGACGGGCTTGAGGTGCTGTCCTTCGAATTCCAGAAGATCAAGTATTCC
TACGATGCCCTGGAGAAGAAGCAGTTTCTCCCCGTGGCCTTTCCTGTGGGAAACGCCTTCTCATACTA
TCAGAGCAACAGAGGCTTCCAGGAAGACTCAGAGATCCGAGCAGCTGAGAAGAAATTTGGGAGCAACA
AGGCCGAGATGGTGGTGCCTGACTTCTCGGAGCTTTTCAAGGAGAGAGCCACAGCCCCCTTCTTTGTA
TTTCAGGCGTTCTGTGTGGGGCTCTGGTGCCTGGATGAGTACTGGTACTACAGCGTCTTTACGCTATC
CATGCTGGTGGCGTTCGAGGCCTCGCTGGTGCAGCAGCAGATGCGGAACATGTCGGAGATCCGGAAGA
TGGGCAACAAGCCCCACATGATCCAGGTCTACCGAAGCCGCAAGTGGAGGCCCATTGCCAGTGATGAG
ATCGTACCAGGGGACATCGTCTCCATCGGCCGCTCCCCACAGGAGAACCTGGTGCCATGTGACGTGCT
TCTGCTGCGAGGCCGCTGCATCGTAGACGAGGCCATGCTCACGGGGGAGTCCGTGCCACAGATGAAGG
AGCCCATCGAAGACCTCAGCCCAGACCGGGTGCTGGACCTCCAGGCTGATTCCCGGCTGCACGTCATC
TTCGGGGGCACCAAGGTGGTGCAGCACATCCCCCCACAGAAAGCCACCACGGGCCTGAAGCCGGTTGA
CAGCGGGTGCGTGGCCTACGTCCTGCGGACCGGATTCAACACATCCCAGGGCAAGCTGCTGCGCACCA
TCCTCTTCGGGGTCAAGAGGGTGACTGCGAACAACCTGGAGACCTTCATCTTCATCCTCTTCCTCCTG
GTGTTTGCCATCGCTGCAGCTGCCTATGTATGGATTGAAGGTACCAAGGACCCCAGCCGGAACCGCTA
CAAGCTGTTTCTGGAGTGCACCCTGATCCTCACCTCGGTCGTGCCTCCTGAGCTGCCCATCGAGCTGT
CCCTGGCCGTCAACACCTCCCTCATCGCCCTGGCCAAGCTCTACATGTACTGCACAGAGCCCTTCCGG
ATCCCCTTTGCTGGCAAGGTCGAGGTGTGCTGCTTTGACAAGACGGGGACGTTGACCAGTGACAGCCT
GGTGGTGCGCGGTGTGGCCGGGCTGAGAGACGGGAAGGAGGTGACCCCAGTGTCCAGCATCCCTGTAG
AAACACACCGGGCCCTGGCCTCGTGCCACTCGCTCATGCAGCTGGACGACGGCACCCTCGTGGGTGAC
CCTCTAGAGAAGGCCATGCTGACGGCCGTGGACTGGACGCTGACCAAAGATGAGAAAGTATTCCCCCG
AAGTATTAAAACTCAGGGGCTGAAAATTCACCAGCGCTTTCATTTTGCCAGTGCCCTGAAGCGAATGT
CCGTGCTTGCCTCGTATGAGAAGCTGGGCTCCACCGACCTTTGTTACATCGCGGCCGTGAAGGGGGCC
CCCGAAACTCTGCACTCCATGGCCCGGGAGGTCAAGCGGGAGGCCCTGGAGTGCAGCCTCAAGTTCGT
CGGCTTCATTGTGGTCTCCTGCCCGCTCAAGGCTGACTCGAAGGCCGTGATCCGGGAGATCCAGAATG
CGTCCCACCGGGTGTTCATGATCACGGGAGACAACCCGCTCACTGCATGCCACGTGGCCCAGGAGCTG
CACTTCATTGAAAAGGCCCACACGCTGATCCTGCAGCCTCCCTCCGAGAAAGGCCGGCAGTGCGAGTG
GCGCTCCATTGACGGCAGCATCGTGCTGCCCCTGGCCCGGGGCTCCCCAAAGGCACTGGCCCTGGAGT
ACGCACTGTGCCTCACAGGCGACGGCTTGGCCCACCTGCAGGCCACCGACCCCCAGCAGCTGCTCCGC
CTCATCCCCCATGTGCAGGTGTTCGCCCGTGTGGCTCCCAAGCAGAAGGAGTTTGTCATCACCAGCCT
GAAGGAGCTGGGCTACGTGACCCTCATGTGTGGGGATGGCACCAACGACGTGGGCGCCCTGAAGCATG
CTGACGTGGGTGTGGCGCTCTTGGCCAATGCCCCTGAGCGGGTTGTCGAGCGGCGACGGCGGCCCCGG
GACAGCCCAACCCTGAGCAACAGTGGCATCAGAGCCACCTCCAGGACAGCCAAGCAGCGGTCGGGGCT
CCCTCCCTCCGAGGAGCAGCCAACCTCCCAGAGGGACCGCCTGAGCCAGGTGCTGCGAGACCTCGAGG
ACGAGAGTACGCCCATTGTGAAACTGGGGGATGCCAGCATCGCAGCACCCTTCACCTCCAAGCTCTCA
TCCATCCAGTGCATCTGCCACGTGATCAAGCAGGGCCGCTGCACGCTGGTGACCACGCTACAGATGTT
CAAGATCCTGGCGCTCAATGCCCTCATCCTGGCCTACAGCCAGAGCGTCCTCTACCTGGAGGGAGTCA
AGTTCAGTGACTTCCAGGCCACCCTACAGGGGCTGCTGCTGGCCGGCTGCTTCCTCTTCATCTCCCGT
TCCAAGCCCCTCAAGACCCTCTCCCGAGAACGGCCCCTGCCCAACATCTTCAACCTGTACACCATCCT
CACCGTCATGCTCCAGTTCTTTGTGCACTTCCTGAGCCTTGTCTACCTGTACCGTGAGGCCCAGGCCC
GGAGCCCCGAGAAGCAGGAGCAGTTCGTGGACTTGTACAAGGAGTTTGAGCCAAGCCTGGTCAACAGC
ACCGTCTACATCATGGCCATGGCCATGCAGATGGCCACCTTCGCCATCAATTACAAAGGCCCGCCCTT
CATGGAGAGCCTGCCCGAGAACAAGCCCCTGGTGTGGAGTCTGGCAGTTTCACTCCTGGCCATCATTG
GCCTGCTCCTCGGCTCCTCGCCCGACTTCAACAGCCAGTTTGGCCTCGTGGACATCCCTGTGGAGTTC
AAGCTGGTCATTGCCCAGGTCCTGCTCCTGGACTTCTGCCTGGCGCTCCTGGCCGACCGCGTCCTGCA
GTTCTTCCTGGGGACCCCGAAGCTGAAAGTGCCTTCCTGAGATGGCAGT

| | | |
|---|---|---|
| NOV66c, SNP13380762 of | SEQ ID NO: 990 | MW at 128721.4kD |

| | | | |
|---|---|---|---|
| CG57562-02, Protein Sequence | SNP Pos: 251 | 1167 aa | SNP Change: Val to Ala |

MAAAAAVGNAVPCGARPCGVRPDGQPKPGPQPRALLAAGPALIANGDELVAAVWPYRRLALLRRLTVL
PFAGLLYPAWLGAAAAGCWGWGSSWVQIPEAALLVLATICLAHALTVLSGHWSVHAHCALTCTPEYDP
SKATFVKVVPTPNNGSTELVALHRNEGEDGLEVLSFEFQKIKYSYDALEKKQFLPVAFPVGNAFSYYQ
SNRGFQEDSEIRAAEKKFGSNKAEMVVPDFSELFKERATAPFFVFQAFCVGLWCLDEYWYYSVFTLSM
LVAFEASLVQQQMRNMSEIRKMGNKPHMIQVYRSRKWRPIASDEIVPGDIVSIGRSPQENLVPCDVLL
LRGRCIVDEANLTGESVPQMKEPIEDLSPDRVLDLQADSRLHVIFGGTKVVQHIPPQKATTGLKPVDS
GCVAYVLRTGFNTSQGKLLRTILFGVKRVTANNLETFIFILFLLVFAIAAAAYVWIEGTKDPSRNRYK
LFLECTLILTSVVPPELPIELSLAVNTSLIALAKLYMYCTEPFRIPFAGKVEVCCFDKTGTLTSDSLV
VRGVAGLRDGKEVTPVSSIPVETHRALASCHSLMQLDDGTLVGDPLEKAMLTAVDWTLTKDEKVFPRS
IKTQGLKIHQRFHFASALKRMSVLASYEKLGSTDLCYIAAVKGAPETLHSMAREVKREALECSLKFVG
FIVVSCPLKADSKAVIREIQNASHRVFMITGDNPLTACHVAQELHFIEKAHTLILQPPSEKGRQCEWR
SIDGSIVLPLARGSPKALALEYALCLTGDGLAHLQATDPQQLLRLIPHVQVFARVAPKQKEFVITSLK
ELGYVTLMCGDGTNDVGALKHADVGVALLANAPERVVERRRRPRDSPTLSNSGIRATSRTAKQRSGLP
PSEEQPTSQRDRLSQVLRDLEDESTPIVKLGDASIAAPFTSKLSSIQCICHVIKQGRCTLVTTLQMFK
ILALNALILAYSQSVLYLEGVKFSDFQATLQGLLLAGCFLFISRSKPLKTLSRERPLPNIFNLYTILT
VMLQFFVHFLSLVYLYREAQARSPEKQEQFVDLYKEFEPSLVNSTVYIMAMANQMATFAINYKGPPFM
ESLPENKPLVWSLAVSLLAIIGLLLGSSPDFNSQFGLVDIPVEFKLVIAQVLLLDFCLALLADRVLQF
FLGTPKLKVPS

| | |
|---|---|
| SEQ ID NO: 991 | 3517 bp |

| | | |
|---|---|---|
| NOV66d, SNP13380787 of | ORF Start: ATG at 5 | ORF Stop: TGA at 3506 |

| | | |
|---|---|---|
| CG57562-02, DNA Sequence | SNP Pos: 2732 | SNP Change: C to A |

AAAGATGGCGGCAGCGGCGGCGGTGGGCAACGCGGTGCCCTGCGGGGCCCGGCCTTGCGGGGTCCGGC
CTGACGGGCAGCCCAAGCCCGGGCCGCAGCCGCGCGCGCTCCTTGCCGCCGGGCCGGCGCTCATAGCG
AACGGTGACGAGCTGGTGGCTGCCGTGTGGCCGTACCGGCGGTTGGCGCTGTTGCGGCGCCTCACGGT
GCTGCCATTCGCCGGGCTGCTTTACCCGGCCTGGTTGGGTGCCGCAGCCGCTGGCTGCTGGGGCTGGG
GCAGCAGTTGGGTGCAGATCCCCGAAGCTGCGCTGCTCGTGCTTGCCACCATCTGCCTCGCGCACGCG
CTCACTGTCCTCTCGGGGCATTGGTCTGTGCACGCGCATTGCGCGCTCACCTGCACCCCGGAGTACGA
CCCCAGCAAAGCGACCTTTGTGAAGGTGGTGCCAACCCCCAACAATGGCTCCACGGAGCTCGTGGCCC
TGCACCGCAATGAGGGCGAAGACGGGCTTGAGGTGCTGTCCTTCGAATTCCAGAAGATCAAGTATTCC
TACGATGCCCTGGAGAAGAAGCAGTTTCTCCCCGTGGCCTTTCCTGTGGGAAACGCCTTCTCATACTA
TCAGAGCAACAGAGGCTTCCAGGAAGACTCAGAGATCCGAGCAGCTGAGAAGAAATTTGGGAGCAACA
AGGCCGAGATGGTGGTGCCTGACTTCTCGGAGCTTTTCAAGGAGAGAGCCACAGCCCCCTTCTTTGTA
TTTCAGGTGTTCTGTGTGGGGCTCTGGTGCCTGGATGAGTACTGGTACTACAGCGTCTTTACGCTATC
CATGCTGGTGGCGTTCGAGGCCTCGCTGGTGCAGCAGCAGATGCGGAACATGTCGGAGATCCGGAAGA
TGGGCAACAAGCCCCACATGATCCAGGTCTACCGAAGCCGCAAGTGGAGGCCCATTGCCAGTGATGAG
ATCGTACCAGGGGACATCGTCTCCATCGGCCGCTCCCCACAGGAGAACCTGGTGCCATGTGACGTGCT
TCTGCTGCGAGGCCGCTGCATCGTAGACGAGGCCATGCTCACGGGGGAGTCCGTGCCACAGATGAAGG
AGCCCATCGAAGACCTCAGCCCAGACCGGGTGCTGGACCTCCAGGCTGATTCCCGGCTGCACGTCATC
TTCGGGGGCACCAAGGTGGTGCAGCACATCCCCCCACAGAAAGCCACCACGGGCCTGAAGCCGGTTGA
CAGCGGGTGCGTGGCCTACGTCCTGCGGACCGGATTCAACACATCCCAGGGCAAGCTGCTGCGCACCA
TCCTCTTCGGGGTCAAGAGGGTGACTGCGAACAACCTGGAGACCTTCATCTTCATCCTCTTCCTCCTG
GTGTTTGCCATCGCTGCAGCTGCCTATGTATGGATTGAAGGTACCAAGGACCCCAGCCGGAACCGCTA
CAAGCTGTTTCTGGAGTGCACCCTGATCCTCACCTCGGTCGTGCCTCCTGAGCTGCCCATCGAGCTGT
CCCTGGCCGTCAACACCTCCCTCATCGCCCTGGCCAAGCTCTACATGTACTGCACAGAGCCCTTCCGG
ATCCCCTTTGCTGGCAAGGTCGAGGTGTGCTGCTTTGACAAGACGGGGACGTTGACCAGTGACAGCCT
GGTGGTGCGCGGTGTGGCCGGGCTGAGAGACGGGAAGGAGGTGACCCCAGTGTCCAGCATCCCTGTAG
AAACACACCGGGCCCTGGCCTCGTGCCACTCGCTCATGCAGCTGGACGACGGCACCCTCGTGGGTGAC
CCTCTAGAGAAGGCCATGCTGACGGCCGTGGACTGGACGCTGACCAAAGATGAGAAAGTATTCCCCCG
AAGTATTAAAACTCAGGGGCTGAAAATTCACCAGCGCTTTCATTTTGCCAGTGCCCTGAAGCGAATGT
CCGTGCTTGCCTCGTATGAGAAGCTGGGCTCCACCGACCTTTGTTACATCGCGGCCGTGAAGGGGGCC
CCCGAAACTCTGCACTCCATGGCCCGGGAGGTCAAGCGGGAGGCCCTGGAGTGCAGCCTCAAGTTCGT
CGGCTTCATTGTGGTCTCCTGCCCGCTCAAGGCTGACTCGAAGGCCGTGATCCGGGAGATCCAGAATG
CGTCCCACCGGGTGTTCATGATCACGGGAGACAACCCGCTCACTGCATGCCACGTGGCCCAGGAGCTG
CACTTCATTGAAAAGGCCCACACGCTGATCCTGCAGCCTCCCTCCGAGAAAGGCCGGCAGTGCGAGTG
GCGCTCCATTGACGGCAGCATCGTGCTGCCCCTGGCCCGGGGCTCCCCAAAGGCACTGGCCCTGGAGT
ACGCACTGTGCCTCACAGGCGACGGCTTGGCCCACCTGCAGGCCACCGACCCCCAGCAGCTGCTCCGC
CTCATCCCCCATGTGCAGGTGTTCGCCCGTGTGGCTCCCAAGCAGAAGGAGTTTGTCATCACCAGCCT
GAAGGAGCTGGGCTACGTGACCCTCATGTGTGGGGATGGCACCAACGACGTGGGCGCCCTGAAGCATG
CTGACGTGGGTGTGGCGCTCTTGGCCAATGCCCCTGAGCGGGTTGTCGAGCGGCGACGGCGGCCCCGG
GACAGCCCAACCCTGAGCAACAGTGGCATCAGAGCCACCTCCAGGACAGCCAAGCAGCGGTCGGGGCT
CCCTCCCTCCGAGGAGCAGCCAACCTCCCAGAGGGACCGCCTGAGCCAGGTGCTGCGAGACCTCGAGG
ACGAGAGTACGACCATTGTGAAACTGGGGGATGCCAGCATCGCAGCACCCTTCACCTCCAAGCTCTCA
TCCATCCAGTGCATCTGCCACGTGATCAAGCAGGGCCGCTGCACGCTGGTGACCACGCTACAGATGTT
CAAGATCCTGGCGCTCAATGCCCTCATCCTGGCCTACAGCCAGAGCGTCCTCTACCTGGAGGGAGTCA
AGTTCAGTGACTTCCAGGCCACCCTACAGGGGCTGCTGCTGGCCGGCTGCTTCCTCTTCATCTCCCGT
TCCAAGCCCCTCAAGACCCTCTCCCGAGAACGGCCCCTGCCCAACATCTTCAACCTGTACACCATCCT
CACCGTCATGCTCCAGTTCTTTGTGCACTTCCTGAGCCTTGTCTACCTGTACCGTGAGGCCCAGGCCC
GGAGCCCCGAGAAGCAGGAGCAGTTCGTGGACTTGTACAAGGAGTTTGAGCCAAGCCTGGTCAACAGC
ACCGTCTACATCATGGCCATGGCCATGCAGATGGCCACCTTCGCCATCAATTACAAAGGCCCGCCCTT
CATGGAGAGCCTGCCCGAGAACAAGCCCCTGGTGTGGAGTCTGGCAGTTTCACTCCTGGCCATCATTG
GCCTGCTCCTCGGCTCCTCGCCCGACTTCAACAGCCAGTTTGGCCTCGTGGACATCCCTGTGGAGTTC
AAGCTGGTCATTGCCCAGGTCCTGCTCCTGGACTTCTGCCTGGCGCTCCTGGCCGACCGCGTCCTGCA
GTTCTTCCTGGGGACCCCGAAGCTGAAAGTGCCTTCCTGAGATGGCAGT

| | | |
|---|---|---|
| NOV66d, SNP13380787 of | SEQ ID NO: 992 | MW at 128753.4kD |

| | | | |
|---|---|---|---|
| CG57562-02, Protein Sequence | SNP Pos: 910 | 1167 aa | SNP Change: Pro to Thr |

MAAAAAVGNAVPCGARPCGVRPDGQPKPGPQPRALLAAGPALIANGDELVAAVWPYRRLALLRRLTVL
PFAGLLYPAWLGAAAAGCWGWGSSWVQIPEAALLVLATICLAHALTVLSGHWSVHAHCALTCTPEYDP
SKATFVKVVPTPNNGSTELVALHRNEGEDGLEVLSFEFQKIKYSYDALEKKQFLPVAFPVGNAFSYYQ
SNRGFQEDSEERAAEKKFGSNKAEMVVPDFSELFKERATAPFFVFQVFCVGLWCLDEYWYYSVFTLSM
LVAFEASLVQQQMRNMSEIRKMGNKPHMIQVYRSRKWRPIASDEIVPGDIVSIGRSPQENLVPCDVLL
LRGRCIVDEAMLTGESVPQMKEPIEDLSPDRVLDLQADSRLHVIFGGTKVVQHIPPQKATTGLKPVDS
GCVAYVLRTGFNTSQGKLLRTILFGVKRVTANNLETFIFILFLLVFAIAAAAYVWIEGTKDPSRNRYK
LFLECTLILTSVVPPELPIELSLAVNTSLIALAKLYMYCTEPFRIPFAGKVEVCCFDKTGTLTSDSLV
VRGVAGLRDGKEVTPVSSIPVETHRALASCHSLMQLDDGTLVGDPLEKAMLTAVDWTLTKDEKVFPRS
IKTQGLKIHQRFHFASALKRMSVLASYEKLGSTDLCYIAAVKGAPETLHSMAREVKREALECSLKFVG
FIVVSCPLKADSKAVIREIQNASHRVFMITGDNPLTACHVAQELHFIEKAHTLILQPPSEKGRQCEWR
SIDGSIVLPLARGSPKALALEYALCLTGDGLAHLQATDPQQLLRLIPHVQVFARVAPKQKEFVITSLK
ELGYVTLMCGDGTNDVGALKHADVGVALLANAPERVVERRRRPRDSPTLSNSGIRATSRTAKQRSGLP
PSEEQPTSQRDRLSQVLRDLEDESTTIVKLGDASIAAPFTSKLSSIQCICHVIKQGRCTLVTTLQMFK
ILALNALILAYSQSVLYLEGVKFSDFQATLQGLLLAGCFLFISRSKPLKTLSRERPLPNIFNLYTILT
VMLQFFVHFLSLVYLYREAQARSPEKQEQFVDLYKEFEPSLVNSTVYIMAMANQMATFAINYKGPPFM
ESLPENKPLVWSLAVSLLAIIGLLLGSSPDFNSQFGLVDIPVEFKLVIAQVLLLDFCLALLADRVLQF
FLGTPKLKVPS

A ClustalW comparison of the above protein sequences yields the following sequence alignment shown in Table 66B.

TABLE 66B
Comparison of the NOV66 protein sequences.

| | |
|---|---|
| NOV66a | MAAAAAVGNAVPCGARPCGVRPDGQPKPGPQPRALLAAGPALIANGDELVAAVWPYRRLA |
| NOV66b | MAAAAAVGNAVPCGARPCGVRPDGQPKPGRSRRALLAAGPALIANGDELVAAVWPYRRLA |
| NOV66a | LLRRLTVLPFAGLLYPAWLGAAAAGCWGWGSSWVQIPEAALLVLATICLAHALTVLSGHW |
| NOV66b | LLRRLTVLPFAGLLYPAWLGAAAAGCWGWGSSWVQIPEAALLVLATICLAHALTVLSGHW |
| NOV66a | SVHAHCALTCTPEYDPSKATFVKVVPTPNNGSTELVALHRNEGEDGLEVLSFEFQKIKYS |
| NOV66b | SVRAHCALTCTPEYDPSKATFVKVVPTPNNGSTELVALHRNEGEDGLEVLSFEFQKIKYS |
| NOV66a | YDALEKKQFLPVAFPVGNAFSYYQSNRGFQEDSEIRAAEKKFGSNKAEMVVPDFSELFKE |
| NOV66b | YDALEKKQFLPVAFPVGNAFSYYQSNRGFQEDSEIRAAEKKFGSNKAEMVVPDFSELFKE |
| NOV66a | RATAPFFVFQVFCVGLWCLDEYWYYSVFTLSMLVAFEASLVQQQMRNMSEIRKMGNKPHM |
| NOV66b | RATAPFFVFQVFCVGLWCLDEYWYYSVFTLSMLVAFEASLVQQQMRNMSEIRKMGNKPHM |
| NOV66a | IQVYRSRKWRPIASDEIVPGDIVSIGRSPQENLVPCDVLLLRGRCIVDEANLTGESVPQM |
| NOV66b | IQVYRSRKWRPIASDEIVPGDIVSIGRSPQENLVPCDVLLLRGRCIVDEAMLTGESVPQM |
| NOV66a | KEPIEDLSPDRVLDLQADSRLHVIFGGTKVVQHIPPQKATTGLKPVDSGCVAYVLRTGFN |
| NOV66b | KEPIEDLSPDRVLDLQADSRLHVIFGGTKVVQHIPPQKATTGLKPVDSGCVAYVLRTGFN |
| NOV66a | TSQGKLLRTILFGVKRVTANNLETFIFILFLLVFAIAAAAYVWIEGTKDPSRNRYKLFLE |
| NOV66b | TSQGKLLRTILFGVKRVTANNLETFIFILFLLVFAIAAAAYVWIEGTKDPSRNRYKLFLE |
| NOV66a | CTLILTSVVPPELPIELSLAVNTSLIALAKLYMYCTEPFRIPFAGKVEVCCFDKTGTLTS |
| NOV66b | CTLILTSVVPPELPIELSLAVNTSLIALAKLYMYCTEPFRIPFAGKVEVCCFDKTGTLTS |
| NOV66a | DSLVVRGVAGLRDGKEVTPVSSIPVETHRALASCHSLMQLDDGTLVGDPLEKAMLTAVDW |
| NOV66b | DSLVVRGVAGLRDGKEVTPVSSIPVETHRALASCHSLMQLDDGTLVGDPLEKAMLTAVDW |
| NOV66a | TLTKDEKVFPRSIKTQGLKIHQRFHFASALKRMSVLASYEKLGSTDLCYIAAVKGAPETL |
| NOV66b | TLTKDEKVFPRSIKTQGLKIHQRFHFASALKRMSVLASYEKLGSTDLCYIAAVKGAPETL |
| NOV66a | HSM-------------------------------------AREVKREALECSLKFVGFIV |
| NOV66b | HSMFSQCPPDYHHIHTEISREGARVLALGYKELGHLTHQQAREVKREALECSLKFVGFIV |
| NOV66a | VSCPLKADSKAVIREIQNASHRVFMITGDNPLTACHVAQELHFIEKAHTLILQPPSEKGR |
| NOV66b | VSCPLKADSKAVIREIQNASHRVVMITGDNPLTACHVAQELHFIEKAHTLILQPPSEKGR |
| NOV66a | QCEWRSIDGSIVLPLARGSPKALALEYALCLTGDGLAHLQATDPQQLLRLIPHVQVFARV |
| NOV66b | QCEWRSIDGSIVLPLARGSPKALALEYALCLTGDGLAHLQATDPQQLLRLIPHVQVFARV |
| NOV66a | APKQKEFVITSLKELGYVTLMCGDGTNDVGALKHADVGVALLANAPERVVERRRRPRDSP |
| NOV66b | APKQKEFVITSLKELGYVTLMCGDGTNDVGALKHADVGVALLANAPERVVERRRRPRDSP |
| NOV66a | TLSNSGIRATSRTAKQRSGLPPSEEQPTSQRDRLSQVLRDLEDESTPIVKLGDASIAAPF |
| NOV66b | TLSNSGIRATSRTAKQRSGLPPSEEQPTSQRDRLSQVLRDLEDESTPIVKLGDASIAAPF |
| NOV66a | TSKLSSIQCICHVIKQGRCTLVTTLQMFKILALNALILAYSQSVLYLEGVKFSDFQATLQ |
| NOV66b | TSKLSSIQCICHVIKQGRCTLVTTLQMFKILAINALILAYSQSVLYLEGVKFSDFQATLQ |
| NOV66a | GLLLAGCFLFISRSKPLKTLSRERPLPNIFNLYTILTVMLQFFVHFLSLVYLYREAQARS |
| NOV66b | GLLLAGCFLFISRSKPLKTLSRERPLPNIFNLYTILTVMLQFFVHFLSLVYLYREAQARS |
| NOV66a | PEKQEQFVDLYKEFEPSLVNSTVYIMAMANQMATFAINYKGPPFMESLPENKPLVWSLAV |
| NOV66b | PEKQEQFVDLYKEFEPSLVNSTVYIMAMANQMATFAINYKGPPFMESLPENKPLVWSLAV |
| NOV66a | SLLAIIGLLLGSSPDFNSQFGLVDIPVEFKLVIAQVLLLDFCLALLADRVLQFFLGTPKL |
| NOV66b | SLLAIIGLLLGSSPDFNSQFGLVDIPVEFKLVIAQVLLLDFCLALLADRVLQFFLGTPKL |
| NOV66a | KVPS |
| NOV66b | KVPS |
| NOV66a | (SEQ ID NO: 986) |
| NOV66b | (SEQ ID NO: 988) |

Further analysis of the NOV66a protein yielded the following properties shown in Table 66C.

TABLE 66C
Protein Sequence Properties NOV66a

| | |
|---|---|
| SignalP analysis: | Cleavage site between residues 16 and 17 |
| PSORT II analysis: | |

| | |
|---|---|
| PSG: | a new signal peptide prediction method |
| | N-region: length 0; pos.chg 0; neg.chg 0 |
| | H-region: length 15; peak value 7.85 |
| | PSG score: 3.45 |
| GvH: | von Heijne's method for signal seq. recognition |
| | GvH score (threshold: −2.1): −4.35 |
| | possible cleavage site: between 46 and 47 |

>>> Seems to have no N-terminal signal peptide

| ALOM: | Klein et al's method for TM region allocation |
|---|---|
| | Init position for calculation: 1 |
| | Tentative number of TMS(s) for the threshold 0.5: 11 |
| | INTEGRAL Likelihood = −7.38 Transmembrane 100-116 |
| | INTEGRAL Likelihood = −2.23 Transmembrane 243-259 |
| | INTEGRAL Likelihood = −0.16 Transmembrane 265-281 |
| | INTEGRAL Likelihood = −11.94 Transmembrane 446-462 |
| | INTEGRAL Likelihood = 0.10 Transmembrane 493-509 |
| | INTEGRAL Likelihood = −0.64 Transmembrane 672-688 |
| | INTEGRAL Likelihood = −0.69 Transmembrane 944-960 |
| | INTEGRAL Likelihood = −1.38 Transmembrane 978-994 |
| | INTEGRAL Likelihood = −3.61 Transmembrane 1017-1033 |
| | INTEGRAL Likelihood = −9.45 Transmembrane 1097-1113 |
| | INTEGRAL Likelihood = −8.65 Transmembrane 1134-1150 |
| | PERIPHERAL Likelihood = 1.01 (at 67) |
| | ALOM score: −11.94 (number of TMSs: 11) |
| MTOP: | Prediction of membrane topology (Hartmann et al.) |
| | Center position for calculation: 107 |
| | Charge difference: 3.0 C( 2.0) - N(−l.0) |
| | C > N: C-terminal side will be inside |

>>> Caution: Inconsistent mtop result with signal peptide
>>> membrane topology: type 3b
MITDISC: discrimination of mitochondrial targeting seq

| | | | |
|---|---|---|---|
| R content: | 2 | Hyd Moment (75): | 2.14 |
| Hyd Moment (95): | 0.67 | G content: | 3 |
| D/E content: | 1 | S/T content: | 0 |
| Score: −5.79 | | | |

Gavel: prediction of cleavage sites for mitochondrial preseq

R-2 motif at 43 PRA|LL

NUCDISC: discrimination of nuclear localization signals

pat4: RRRR (5) at 855
pat4: RRRP (4) at 856
pat4: RRPR (4) at 857
pat7: none
bipartite: RKMGNKPHMIQVYRSRK at 292
content of basic residues: 10.4%
NLS Score: 0.83

KDEL: ER retention motif in the C-terminus: none
ER Membrane Retention Signals:

KKXX-like motif in the C-terminus: LKVP
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

--------------------------
Final Results (k = 9/23):

77.8%: endoplasmic reticulum
11.1%: mitochondrial
11.1%: vacuolar

>> prediction for CG57562-02 is end (k = 9)

A search of the NOV66a protein against the Geneseq database, a proprietary database that contains sequences published in patents and patent publication, yielded several homologous proteins shown in Table 66D.

TABLE 66D
Geneseq Results for NOV66a

| | | NOV66a | Identities/ | |
|---|---|---|---|---|
| | | Residues/ | Similarities for | Ex- |
| Geneseq | Protein/Organism/Length | Match | the Matched | pect |
| Identifier | [Patent #, Date] | Residues | Region | Value |
| AAU91186 | Human HEAT-3 polypeptide - | 1 . . . 1167 | 1165/1204 (96%) | 0.0 |
| | Homo sapiens, 1204 aa. | 1 . . . 1204 | 1165/1204 (96%) | |
| | [WO200216591-A2, 28-Feb.- | | | |
| | 2002] | | | |
| AAE16780 | Human transporter and ion | 1 . . . 1167 | 1159/1204 (96%) | 0.0 |
| | channel-17 (TRIGH-17) protein - | 1 . . . 1197 | 1159/1204 (96%) | |
| | Homo sapiens, 1197 aa. | | | |
| | [WO200192304-A2, 06-Dec.- | | | |
| | 2001] | | | |
| ABG30096 | Novel human diagnostic protein | 328 . . . 1167 | 829/915 (90%) | 0.0 |
| | #30087 - Homo sapiens, 914 aa. | 7 . . . 914 | 830/915 (90%) | |
| | [WO200175067-A2, 11-Oct.- | | | |
| | 2001] | | | |
| AAB42279 | Human ORFX ORF2043 | 418 . . . 1167 | 734/789 (93%) | 0.0 |
| | polypeptide sequence SEQ ID | 1 . . . 789 | 737/789 (93%) | |
| | NO:4086 - Homo sapiens, 789 | | | |
| | aa. [WO200058473-A2, 05- | | | |
| | Oct.-2000] | | | |
| AAM93412 | Human polypeptide, SEQ ID | 633 . . . 1167 | 530/572 (92%) | 0.0 |
| | NO: 3024 - Homo sapiens, 572 | 1 . . . 572 | 530/572 (92%) | |
| | aa. [EP1130094-A2, 05-Sep.- | | | |
| | 2001] | | | |

In a BLAST search of public sequence databases, the NOV66a protein was found to have homology to the proteins shown in the BLASTP data in Table 66E.

TABLE 66E
Public BLASTP Results for NOV66a

| | | NOV66a | Identities/ | |
|---|---|---|---|---|
| Protein | | Residues/ | Similarities for | Ex- |
| Accession | | Match | the Matched | pect |
| Number | Protein/Organism/Length | Residues | Portion | Value |
| Q9HD20 | Probable cation-transporting | 1..1167 | 1166/1204 (96%) | 0.0 |
| | ATPase 2 (EC 3.6.3.-) (CGI- | 1 . . . 1204 | 1166/1204 (96%) | |
| | 152) - Homo sapiens (Human), | | | |
| | 1204 aa. | | | |
| CAD29018 | Sequence 8 from Patent | 1 . . . 1167 | 1165/1204 (96%) | 0.0 |
| | WO0216591 - Homo sapiens | 1 . . . 1204 | 1165/1204 (96%) | |
| | (Human), 1204 aa. | | | |
| Q9EPE9 | Probable cation-transporting | 5 . . . 1167 | 1103/1200 (91%) | 0.0 |
| | ATPase 2 (EC 3.6.3.-) (CATP) - | 2 . . . 1200 | 1127/1200 (93%) | |
| | Mus musculus (Mouse), 1200 | | | |
| | aa. | | | |
| Q9VKJ6 | BCDNA:GH06032 protein - | 98 . . . 1164 | 594/1123 (52%) | 0.0 |
| | Drosophila melanogaster (Fruit | 105 . . . 1219 | 770/1123 (67%) | |
| | fly), 1225 aa. | | | |
| Q8NC73 | Hypothetical protein FLJ90439 - | 633 . . . 1167 | 530/572 (92%) | 0.0 |
| | Homo sapiens (Human), 572 | 1 . . . 572 | 530/572 (92%) | |
| | aa. | | | |

PFam analysis indicates that the NOV66a protein contains the domains shown in the Table 66F.

TABLE 66F
Domain Analysis of NOV66a

| | | Identities/ | |
|---|---|---|---|
| | NOV66a | Similarities | |
| | Match | for the | |
| | Region | Matched Region | Expect Value |
| E1-E2_ATPase | 266..511 | 62/284 (22%) | 7.4e-05 |
| | | 166/284 (58%) | |
| Hydrolase | 527 . . . 848 | 35/327 (11%) | 2.8e-06 |
| | | 199/327 (61%) | |

### Example 67

The NOV67 clone was analyzed, and the nucleotide and encoded polypeptide sequences are shown in Table 67A.

TABLE 67A
NOV67 Sequence Analysis

| | | |
|---|---|---|
| NOV67a, CG57758-03 | SEQ ID NO: 993 | 3147 bp |

| | | |
|---|---|---|
| DNA Sequence | ORF Start: ATG at 2 | ORF Stop: TAG at 1706 |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGTCTCGCTCCATCGGCCTCAATCCGCTGTACATCATGCTGCCCTGTACCCTGAGTGCCT
CCTTTGCCTTCATGTTGCCTGTGGCCACCCCTCCAAATGCCATCGTGTTCACCTATGGGCACCTCAAG
GTTGCTGACATGGTGAAAACAGGAGTCATAATGAACATAATTGGAGTCTTCTGTGTGTTTTTGGCTGT
CAACACCTGGGGACGGGCCATATTTGACTTGGATCATTTCCCTGACTGGGCTAATGTGACACATATTG
AGACTTAGGAAGAGCCACAAGACCACACACACAGCCCTTACCCTCCTCAGGACTACCGAACCTTCTGG
CACACCTTGTACAGAGTTTTGGGGTTCACACCCCAAAATGACCCAACGATGTCCACACACCACCAAAA
CCCAGCCAATGGGCCACCTCTTCCTCCAAGCCCAGATGCAGAGATGGTCATGGGCAGCTGGAGGGTAG
GCTCAGAAATGAAGGGAACCCCTCAGTGGGCTGCTGGACCCATCTTTCCCAAGCCTTGCCATTATCTC
TGTGAGGGAGGCCAGGTAGCCGAGGGATCAGGATGCAGGCTGCTGTACCCGCTCTGCCTCAAGCATCC
CCCACACAGGGCTCTGGTTTTCACTCGCTTCGTCCTAGATAGTTTAAATGGGAATCGGATCCCCTGGT
TGAGAGCTAAGACAACCACCTACCAGTGCCCATGTCCCTTCCAGCTCACCTTGAGCAGCCTCAGATCA
TCTCTGTCACTCTGGAAGGGACACCCCAGCCAGGGACGGAATGCCTGGTCTTGAGCAACCTCCCACTG
CTGGAGTGCGAGTGGGAATCAGAGCCTCCTGAAGCCTCTGGGAACTCCTCCTGTGGCCACCACCAAAG
GATGAGGAATCTGAGTTGCCAACTTCAGGACGACACCTGGCTTGCCACCCACAGTGCACCACAGGCCA
ACCTACGCCCTTCATCACTTGGTTCTGTTTTAATCGACTGGCCCCCTGTCCCACCTCTCCAGTGAGCC
TCCTTCAACTCCTTGGTCCCCTGTTGTCTGGGTCAACATTTGCCGAGACGCCTTGGCTGGCACCCTCT
GGGGTCCCCCTTTTCTCCCAGGCAGGTCATCTTTTCTGGGAGATGCTTCCCCTGCCATCCCCAAATAG
CTAGGATCACACTCCAAGTATGGGCAGTGATGGCGCTCTGGGGGCCACAGTGGGCTATCTAGGCCCTC
CCTCACCTGAGGCCCAGAGTGGACACAGCTGTTAATTTCCACTGGCTATGCCACTTCAGAGTCTTTCA
TGCCAGCGTTTGAGCTCCTCTGGGTAAAATCTTCCCTTTGTTGACTGGCCTTCACAGCCATGGCTGGT
GACAACAGAGGATCGTTGAGATTGAGCAGCGCTTGGTGATCTCTCAGCAAACAACCCCTGCCCGTGGG
CCAATCTACTTGAAGTTACTCGGACAAAGACCCCAAAGTGGGGCAACAACTCCAGAGAGGCTGTGGGA
ATCTTCAGAAGCCCCCCTGTAAGAGACAGACATGAGAGACAAGCATCTTCTTTCCCCCGCAAGTCCAT
TTTATTTCCTTCTTGTGCTGCTCTGGAAGAGAGGCAGTAGCAAAGAGATGAGCTCCTGGATGGCATTT
TCCAGGGCAGGAGAAAGTATGAGAGCCTCAGGAAACCCCATCAAGGACCGAGTATGTGTCTGGTTCCT
TTGGTGGTTGGCTTCTGGC

NOV67a, CG57758-03

| | | | |
|---|---|---|---|
| Protein Sequence | SEQ ID NO: 994 | 568 aa | MW at 63061.4kD |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLELVDKGKAKELPGSQVIFEGPTLGQOEDQERKRL
CKANTLCICYAASIGGTATLTGTGPNVVLLGQNNELFPDSKDLVNFASWFAFAFPNMLVNLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMSRSIGLNPLYIMLPCTLSASFAFMLPVATPPNAIVFTYGHLKVADMVKTGVIMNIIGVFCVFLAV
NTWGRAIFDLDHFPDWANVTHIET

| | | |
|---|---|---|
| NOV66b, 308537854 | SEQ ID NO: 995 | 1729 bp |

| | | |
|---|---|---|
| DNA Sequence | ORF Start: at 2 | ORF Stop: end of sequence |

CACCAGATCTCCCACCATGGCCTCGGCGCTGAGCTATGTCTCCAAGTTCAAGTCCTTCGTGATCTTGT
TCGTCACCCCGCTCCTGCTGCTGCCACTCGTCATTCTGATGCCCGCCAAGTTTGTCAGGTGTGCCTAC
GTCATCATCCTCATGGCCATTTACTGGTGCACAGAAGTCATCCCTCTGGCTGTCACCTCTCTCATGCC
TGTCTTGCTTTTCCCACTCTTCCAGATTCTGGACTCCAGGCAGGTGTGTGTCCAGTACATGAAGGACA
CCAACATGCTGTTCCTGGGCGGCCTCATCGTGGCCGTGGCTGTGGAGCGCTGGAACCTGCACAAGAGG
ATCGCCCTGCGCACGCTCCTCTGGGTGGGGGCCAAGCCTGCACGGCTGATGCTGGGCTTCATGGGCGT
CACAGCCCTCCTGTCCATGTGGATCAGTAACACGGCAACCACGGCCATGATGGTGCCCATCGTGGAGG
CCATATTGCAGCAGATGGAAGCCACAAGCGCAGCCACCGAGGCCGGCCTGGAGCTGGTGGACAAGGGC
AAGGCCAAGGAGCTGCCAGGGAGTCAAGTGATTTTTGAAGGCCCCACTCTGGGGCAGCAGGAAGACCA
AGAGCGGAAGAGGTTGTGTAAGGCCATGACCCTGTGCATCTGCTACGCGGCCAGCATCGGGGGCACCG
CCACCCTGACCGGGACGGGACCCAACGTGGTGCTCCTGGGCCAGATGAACGAGTTGTTTCCTGACAGC
AAGGACCTCGTGAACTTTGCTTCCTGGTTTGCATTTGCCTTTCCCAACATGCTGGTGATGCTGCTGTT
CGCCTGGCTGTGGCTCCAGTTTGTTTACATGAGATTCAATTTTAAAAAGTCCTGGGGCTGCGGGCTAG
AGAGCAAGAAAAACGAGAAGGCTGCCCTCAAGGTGCTGCAGGAGGAGTACCGGAAGTTGGGGCCCTTG
TCCTTCGCGGAGATCAACGTGCTGATCTGCTTCTTCCTGCTGGTCATCCTGTGGTTCTCCCGAGACCC
CGGCTTCATGCCCGGCTGGCTGACTGTTGCCTGGGTGGAGGGTGAGACAAAGTATGTCTCCGATGCCA
CTGTGGCCATCTTTGTGGCCACCCTGCTATTCATTGTGCCTTCACAGAAGCCCAAGTTTAACTTCCGC
AGCCAGACTGAGGAAGAAAGGAAAACTCCATTTTATCCCCCTCCCCTGCTGGATTGGAAGGTAACCCA
GGAGAAAGTGCCCTGGGGCATCGTGCTGCTACTAGGGGGCGGATTTGCTCTGGCTAAAGGATCCGAGG
CCTCGGGGCTGTCCGTGTGGATGGGGAAGCAGATGGAGCCCTTGCACGCAGTGCCCCCGGCAGCCATC
ACCTTGATCTTGTCCTTGCTCGTTGCCGTGTTCACTGAGTGCACAAGCAACGTGGCCACCACCACCTT
GTTCCTGCCCATCTTTGCCTCCATGTCTCGCTCCATCGGCCTCAATCCGCTGTACATCATGCTGCCCT
GTACCCTGAGTGCCTCCTTTGCCTTCATGTTGCCTGTGGCCACCCCTCCAAATGCCATCGTGTTCACC
TATGGGCACCTCAAGGTTGCTGACATGGTGAAAACAGGAGTCATAATGAACATAATTGGAGTCTTCTG
TGTGTTTTTGGCTGTCAACACCTGGGGACGGGCCATATTTGACTTGGATCATTTCCCTGACTGGGCTA
ATGTGACACATATTGAGACTCTCGAGGGC

NOV67b, 308537854

| | | | |
|---|---|---|---|
| Protein Sequence | SEQ ID NO: 996 | 576 aa | MW at 63903.4kD |

TRSPTMASALSYVSKFKSFVILFVTPLLLLPLVILMPAKFVRCAYVIILMAIYWCTEVIPLAVTSLMP
VLLFPLFQILDSRQVCVQYMKDTNMLFLGGLIVAVAVERWNLHKRIALRTLLWVGAKPARLMLGFMGV
TALLSMWISNTATTANNVPIVEAILQQMEATSAATEAGLELVDKGKAKELPGSQVIFEGPTLGQQEDQ
ERKRLCKAMTLCICYAASIGGTATLTGTGPNVVLGQMNELFPDSKDLVNFASWFAFAFPPNMLVMLLF
AWLWLQFVYNRFNFKKSWGCGLESKKNEKAALKVLQEEYRKLGPLSFAEINVLICFFLLVILWFSRDP
GFMPGWLTVAWVEGETKYVSDATVAIFVATLLFIVPSQKPKFNFRSQTEEERKTPFYPPPLLDWKVTQ
EKVPWGIVLLLGGGFALAKGSEASGLSVWMGKQMEPLHAVPPAAITLILSLLVAVFTECTSNVATTTL
FLPIFASMSRSIGLNPLYIMLPCTLSASFAFMLPVATPPNAIVFTYGHLKVADMVKTGVIMNIIGVFC
VFLAVNTWGRAIFDLDHFPDWANVTHIETLEG

| | | |
|---|---|---|
| NOV67c, CG57758-01 | SEQ ID NO: 997 | 1790 bp |

| | | |
|---|---|---|
| DNA Sequence | ORF Start: ATG at 16 | ORF Stop: TAG at 1720 |

TCTCCCTCCCGCGCGATGGCCTCGGCGCTGAGCTATGTCTCCAAGTTCAAGTCCTTCGTGATCTTGTT
CGTCACCCCGCTCCTGCTGCTGCCACTCGTCATTCTGATGCCCGCCAAGGTCAGTTGTGCCTACGTCA
TCATCCTCATGGCCATTTACTGGTGCACAGAAGTCATCCCTCTGGCTGTCACCTCTCTCATGCCTGTC
TTGCTTTTCCCACTCTTCCAGATTCTGGACTCCAGGCAGGTGTGTGTCCAGTACATGAAGGACACCAA
CATGCTGTTCCTGGGCGGCCTCATCGTGGCCGTGGCTGTGGAGCGCTGGAACCTGCACAAGAGGATCG
CCCTGCGCACGCTCCTCTGGGTGGGGGCCAAGCCTGCACGGCTGATGCTGGGCTTCATGGGCGTCACA
GCCCTCCTGTCCATGTGGATCAGTAACACGGCAACCACGGCCATGATGGTGCCCATCGTGGAGGCCAT
ATTGCAGCAGATGGAAGCCACAAGCGCAGCCACCGAGGCCGGCCTGGAGCTGGTGGACAAGGGCAAGG
CCAAGGAGCTGCCAGGGAGTCAAGTGATTTTTGAAGGCCCCACTCTGGGGCAGCAGGAAGACCAAGAG
CGGAAGAGGTTGTGTAAGGCCATGACCCTGTGCATCTGCTACGCGGCCAGCATCGGGGGCACCGCCAC
CCTGACCGGGACGGGACCCAACGTGGTGCTCCTGGGCCAGATGAACGAGTTGTTTCCTGACAGCAAGG
ACCTCGTGAACTTTGCTTCCTGGTTTGCATTTGCCTTTCCCAACATGCTGGTGATGCTGCTGTTCGCC
TGGCTGTGGCTCCAGTTTGTTTACATGTTCTCCAGTTTTAAAAAGTCCTGGGGCTGCGGGCTAGAGAG
CAAGAAAAACGAGAAGGCTGCCCTCAAGGTGCTGCAGGAGGAGTACCGGAAGCTGGGGCCCTTGTCCT
TCGCGGAGATCAACGTGCTGATCTGCTTCTTCCTGCTGGTCATCCTGTGGTTCTCCCGAGACCCCGGC
TTCATGCCCGGCTGGCTGACTGTTGCCTGGGTGGAGGGTGAGACAAAGTATGTCTCCGATGCCACTGT
GGCCATCTTTGTGGCCACCCTGCTATTCATTGTGCCTTCACAGAAGCCCAAGTTTAACTTCCGCAGCC
AGACTGAGGAAGGTAAGTCTCCTGTTCTGATCGCCCCCCCTCCCCTGCTGGATTGGAAGGTAACCCAG
GAGAAAGTGCCCTGGGGCATCGTGCTGCTACTAGGGGGCGGATTTGCTCTGGCTAAAGGATCCGAGGC
CTCGGGGCTGTCCGTGTGGATGGGGAAGCAGATGGAGCCCTTGCACGCAGTGCCCCCGGCAGCCATCA
CCTTGATCTTGTCCTTGCTCGTTGCCGTGTTCACTGAGTGCACAAGCAACGTGGCCACCACCACCTTG
TTCCTGCCCATCTTTGCCTCCATGTCTCGCTCCATCGGCCTCAATCCGCTGTACATCATGCTGCCCTG
TACCCTGAGTGCCTCCTTTGCCTTCATGTTGCCTGTGGCCACCCCTCCAAATGCCATCGTGTTCACCT
ATGGGCACCTCAAGGTTGCTGACATGGTGAAAACAGGAGTCATAATGAACATAATTGGAGTCTTCTGT
GTGTTTTTGGCTGTCAACACCTGGGGACGGGCCATATTTGACTTGGATCATTTCCCTGACTGGGCTAA
TGTGACACATATTGAGACTTAGGAAGAGCCACAAGACCACACACACAGCCCTTACCCTCCTCAGGACT
ACCGAACCTTCTGGCACACCTT

NOV67c, CG57758-01

| | | | |
|---|---|---|---|
| Protein Sequence | SEQ ID NO: 998 | 568 aa | MW at 62592.9kD |

MASALSYVSKFKSFVILFVTPLLLLPLVILMPAKVSCAYVIILMAIYWCTEVIPLAVTSLMPVLLFPL
FQILDSRQVCVQYMKDTNMLFLGGLIVAVAVERWNLHKRIALRTLLWVGAKPARLMLGFMGVTALLSM
WISNTATTANMVPIVEAILQQMEATSAATEAGLELVDKGKAKELPGSQVIFEGPTLGQQEDQERKRLC
KAMTLCICYAASIGGTATLTGTGPNVVLLGQMNELFPDSKDLVNFASWFAFAFPNNLVMLLFAWLWLQ
FVYMFSSFKKSWGCGLESKKNEKAALKVLQEEYRKLGPLSFAEINVLICFFLLVILWFSRDPGFMPGW
LTVAWVEGETKYVSDATVAIFVATLLFIVPSQKPKFNFRSQTEEGKSPVLIAPPPLLDWKVTQEKVPW
GIVLLLGGGFALAKGSEASGLSVWMGKQMEPLHAVPPAAITLILSLLVAVFTECTSNVATTTLFLPIF
ASMSRSIGLNPLYIMLPCTLSASFAFMLPVATPPNAIVFTYGHLKVADMVKTGVIMNIIGVFCVFLAV
NTWGRAIFDLDHFPDWANVTHIET

| | | |
|---|---|---|
| NOV67d, CG57758-02 | SEQ ID NO: 999 | 1899 bp |

| | | |
|---|---|---|
| DNA Sequence | ORF Start: ATG at 31 | ORF Stop: TAG at 1879 |

CGTCTCGCCCGCCAGTCTCCCTCCCGCGCGATGGCCTCGGCGCTGAGCTATGTCTCCAAGTTCAAGTC
CTTCGTGATCTTGTTCGTCACCCCGCTCCTGCTGCTGCCACTCGTCATTCTGATGCCCGCCAAGGTCA
GTTGCTGTGCCTACGTCATCATCCTCATGGCCATTTACTGGTGCACAGAAGTCATCCCTCTGGCTGTC
ACCTCTCTCATGCCTGTCTTGCTTTTCCCACTCTTCCAGATTCTGGACTCCAGGCAGGTGTGTGTCCA
GTACATGAAGGACACCAACATGCTGTTCCTGGGCGGCCTCATCGTGGCCGTGGCTGTGGAGCGCTGGA
ACCTGCACAAGAGGATCGCCCTGCGCACGCTCCTCTGGGTGGGGGCCAAGCCTGCACGGCTGATGCTG
GGCTTCATGGGCGTCACAGCCCTCCTGTCCATGTGGATCAGTAACACGGCAACCACGGCCATGATGGT
GCCCATCGTGGAGGCCATATTGCAGCAGATGGAAGCCACAAGCGCAGCCACCGAGGCCGGCCTGGAGG
GACAAGGTACCACAATAAACAACCTGAATGCACTGGAGGATGATACAGTGAAAGCAGTACTAGGAGGA
AAGTGTGTAGCTATAATAAGCACTTACGTCAAAAAAGTAGAAAAACTTCAAATAAACAATCTAATGAC
ACCTCTTAAAAAACTAGAAAAGCAAGAGCAACAGGACCTAGGGCCTGGCATCAGGCCTCAGGACTCTG
CCCAGTGCCAGGAAGACCAAGAGCGGAAGAGGTTGTGTAAGGCCATGACCCTGTGCATCTGCTACGCG
GCCAGCATCGGGGGCACCGCCACCCTGACCGGGACGGGACCCAACGTGGTGCTCCTGGGCCAGATGAA
CGAGTTGTTTCCTGACAGCAAGGACCTCGTGAACTTTGCTTCCTGGTTTGCATTTGCCTTTCCCAACA
TGCTGGTGATGCTGCTGTTCGCCTGGCTGTGGCTCCAGTTTGTTTACATGTTCTCCAGTTTTAAAAAG
TCCTGGGGCTGCGGGCTAGAGAGCAAGAAAAACGAGAAGGCTGCCCTCAAGGTGCTGCAGGAGGAGTA
CCGGAAGCTGGGGCCCTTGTCCTTCGCGGAGATCAACGTGCTGATCTGCTTCTTCCTGCTGGTCATCC
TGTGGTTCTCCCGAGACCCCGGCTTCATGCCCGGCTGGCTGACTGTTGCCTGGGTGGAGGGTGAGACA
AAGTCAGTCTCCGATGCCACTGTGGCCATCTTTGTGGCCACCCTGCTATTCATTGTGCCTTCACAGAA
GCCCAAGTTTAACTTCCGCAGCCAGACTGAGGAAGGTAAGTCTCCTGTTCTGATCGCCCCCCCTCCCC
TGCTGGATTGGAAGGTAACCCAGGAGAAAGTGCCCTGGGGCATCGTGCTGCTACTAGGGGGCGGATTT
GCTCTGGCTAAAGGATCCGAGGCCTCGGGGCTGTCCGTGTGGATGGGGAAGCAGATGGAGCCCTTGCA
CGCAGTGCCCCCGGCAGCCATCACCTTGATCTTGTCCTTGCTCGTTGCCGTGTTCACTGAGTGCACAA
GCAACGTGGCCACCACCACCTTGTTCCTGCCCATCTTTGCCTCCATGTCTCGCTCCATCGGCCTCAAT
CCGCTGTACATCATGCTGCCCTGTACCCTGAGTGCCTCCTTTGCCTTCATGTTGCCTGTGGCCACCCC
TCCAAATGCCATCGTGTTCACCTATGGGCACCTCAAGGTTGCTGACATGGTAAAAACAGGAGTCATAA
TGAACATAATTGGAGTCTTCTGTGTGTTTTTGGCTGTCAACACCTGGGGACGGGCCATATTTGACTTG
GATCATTTCCCTGACTGGGCTAATGTGACACATATTGAGACTTAGGAAGAGCCACAAGACCAC

NOV67d, CG57758-02

| | | | |
|---|---|---|---|
| Protein Sequence | SEQ ID NO: 1000 | 616 aa | MW at 67816.9kD |

MASALSYVSKFKSFVILFVTPLLLLPLVILMPAKVSCCAYVIILMAIYWCTEVIPLAVTSLMPVLLFP
LFQILDSRQVCVQYMKDTNMLFLGGLIVAVAVERWNLHKRIALRTLLWVGAKPARIMLGFMGVTALLS
MWISNTATTANMVPIVEAILQQMEATSAATEAGLEGQGTTINNLNALEDDTVKAVLGGKCVAIISTYV
KKVEKLQINNLMTPLKKLEKQEQQDLGPGIRPQDSAQCQEDQERKRLCKAMTLCICYAASIGGTATLT
GTGPNVVLLGQMNELFPDSKDLVNFASWFAFAFPNMLVMLLFAWLWLQFVYMFSSFKKSWGCGLESKK
NEKAALKVLQEEYRKLGPLSFAEINVLICFFLLVILWFSRDPGFMPGWLTVAWVEGETKSVSDATVAI
FVATLLFIVPSQKPKFNFRSQTEEGKSPVLIAPPPLLDWKVTQEKVPWGIVLLLGGGFALAKGSEASG
LSVWMGKQMEPLHAVPPAAITLILSLLVAVFTECTSNVATTTLFLPIFASMSRSIGLNPLYIMLPCTL
SASFAFMLPVATPPNAIVFTYGHLKVADMVKTGVIMNIIGVFCVFLAVNTWGRAIFDLDHFPDWANVT
HIET

| | | |
|---|---|---|
| NOV67e, CG57758-04 | SEQ ID NO: 1001 | 1606 bp |

| | | |
|---|---|---|
| DNA Sequence | ORF Start: ATG at 2 | ORF Stop: TAG at 1568 |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGGTGAAAACAGGAGTCATAATGAACATAATTGGAGTCTTCTGTGTGTTTTTGGCTGTCA
ACACCTGGGGACGGGCCATATTTGACTTGGATCATTTCCCTGACTGGGCTAATGTGACACATATTGAG
ACTTAGGAAGAGCCACAAGACCACACACATAGCCCTTACCCT

NOV67e, CG57758-04

| | | | |
|---|---|---|---|
| Protein Sequence | SEQ ID NO: 1002 | 522 aa | MW at 58109.6kD |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLELVDKGKAKELPGSQVIFEGPTLGQQEDQERKRL
CKANTLCICYAASIGGTATLTGTGPNVVLLGQMNELFPDSKDLVNFASWFAFAFPNMLVMLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMVKTGVIMNIIGVFCVFLAVNTWGRAIFDLDHFPDWANVTHIET

| | | |
|---|---|---|
| NOV67f, CG57758-05 | SEQ ID NO: 1003 | 1781 bp |

| | | |
|---|---|---|
| DNA Sequence | ORF Start: ATG at 2 | ORF Stop: TGA at 1550 |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGAATCACGTCCCCAAGAGCTTCTGTGTTCTGTACGGTGATGTTGCAGTGCTGTCTTTCC
GCAGTCTCGCTCCATCGGCCTCAATCCGCTGTACATCATGCTGCCCTGTACCCTGAGTGCCTCCTTTG
CCTTCATGTTGCCTGTGGCCACCCCTCCAAATGCCATCGTGTTCACCTATGGGCACCTCAAGGTTGCT
GACATGGTGAAAACAGGAGTCATAATGAACATAATTGGAGTCTTCTGTGTGTTTTTGGCTGTCAACAC
CTGGGGACGGGCCATATTTGACTTGGATCATTTCCCTGACTGGGCTAATGTGACACATATTGAGACTT
AGGAAGAGCCACA

NOV67f, CG57758-05

| | | | |
|---|---|---|---|
| Protein Sequence | SEQ ID NO: 1004 | 516 aa | MW at 57173.5kD |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTANMVPIVEAILQQMEATSAATEAGLELVDKGKAKELPGSQVIFEGPTLGQQEDQERKRL
CKAMTLCICYAASIGGTATLTGTGPNVVLLGQMNELFPDSKDLVNFASWFAFAFPNMLVMLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMNHVPKSFCVLYGDVAVLSFRSLAPSASIRCTSCCPVP

A ClustalW comparison of the above protein sequences yields the following sequence alignment shown in Table 67B.

TABLE 67B
Comparison of the NOV67 protein sequences.

| | |
|---|---|
| NOV67a | -----MASALSYVSKFKSFVILFVTPLLLLPLVILMPAKFVRCAYVIILMAIYWCTEVIP |
| NOV67b | TRSPTMASALSYVSKFKSFVILFVTPLLLLPLVILMPAKFVRCAYVIILMAIYWCTEVIP |
| NOV67c | -----MASALSYVSKFKSFVILFVTPLLLLPLVILMPAKVS-CAYVIILMAIYWCTEVIP |
| NOV67d | -----MASALSYVSKFKSFVILFVTPLLLLPLVILMPAKVSCCAYVIILMAIYWCTEVIP |
| NOV67e | -----MASALSYVSKFKSFVILFVTPLLLLPLVILMPAKFVRCAYVIILMAIYWCTEVIP |
| NOV67f | -----MASALSYVSKFKSFVILFVTPLLLLPLVILMPAKFVRCAYVIILMAIYWCTEVIP |
| NOV67a | LAVTSLMPVLLFPLFQILDSRQVCVQYMKDTNMLFLGGLIVAVAVERWNLHKRIALRTLL |
| NOV67b | LAVTSLMPVLLFPLFQILDSRQVCVQYNKDTNMLFLGGLIVAVAVERWNLHKRIALRTLL |
| NOV67c | LAVTSLMPVLLFPLFQILDSRQVCVQYMKDTNMLFLGGLIVAVAVERWNLHKRIALRTLL |
| NOV67d | LAVTSLMPVLLFPLFQILDSRQVCVQYMKDTNMLFLGGLIVAVAVERWNLHKRIALRTLL |
| NOV67e | LAVTSLMPVLLFPLFQILDSRQVCVQYMKDTNMLFLGGLIVAVAVERWNLHKRIALRTLL |
| NOV67f | LAVTSLMPVLLFPLFQILDSRQVCVQYMKDTNMLFLGGLIVAVAVERWNLHKRIALRTLL |
| NOV67a | WVGAKPARLMLGFMGVTALLSMWISNTATTAMMVPIVEAILQQMEATSAATEAGLELVDK |
| NOV67b | WVGAKPARLMLGFMGVTALLSMWISNTATTAMMVPIVEAILQQMEATSAATEAGLELVDK |
| NOV67c | WVGAKPARLMLGFMGVTALLSMWISNTATTAMMVPIVEAILQQMEATSAATEAGLELVDK |
| NOV67d | WVGAKPARLMLGFMGVTALLSMWISNTATTAMMVPIVEAILQQMEATSAATEAGLEGQGT |
| NOV67e | WVGAKPARLMLGFMGVTALLSMWISNTATTAMMVPIVEAILQQMEATSAATEAGLELVDK |
| NOV67f | WVGAKPARLMLGFMGVTALLSMWISNTATTAMMVPIVEAILQQMEATSAATEAGLELVDK |
| NOV67a | GK-----------------------------------------AKELPGSQVIFEGPTLG |
| NOV67b | GK-----------------------------------------AKELPGSQVIFEGPTLG |
| NOV67c | GK-----------------------------------------AKELPGSQVIFEGPTLG |
| NOV67d | TINNLNALEDDTVKAVLGGKCVAIISTYVKKVEKLQINNLMTPLKKLEKQEQQDLGPGIR |
| NOV67e | GK-----------------------------------------AKELPGSQVIFEGPTLG |
| NOV67f | GK-----------------------------------------AKELPGSQVIFEGPTLG |
| NOV67a | -Q-----QEDQERKRLCKAMTLCICYAASIGGTATLTGTGPNVVLLGQMNELFPDSKDLV |
| NOV67b | -Q-----QEDQERKRLCKAMTLCICYAASIGGTATLTGTGPNVVLLGQMNELFPDSKDLV |
| NOV67c | -Q-----QEDQERKRLCKAMTLCICYAASIGGTATLTGTGPNVVLLGQMNELFPDSKDLV |
| NOV67d | PQDSAQCQEDQERKRLCKAMTLCICYAASIGGTATLTGTGPNVVLLGQMNELFPDSKDLV |
| NOV67e | -Q-----QEDQERKRLCKANTLCICYAASIGGTATLTGTGPNVVLLGQMNELFPDSKDLV |
| NOV67f | -Q-----QEDQERKRLCKAMTLCICYAASIGGTATLTGTGPNVVLLGQMNELFPDSKDLV |
| NOV67a | NFASWFAFAFPNMLVMLLFAWLWLQFVYMRFNFKKSWGCGLESKKNEKAALKVLQEEYRK |
| NOV67b | NFASWFAFAFPNMLVMLLFAWLWLQFVYMRFNFKKSWGCGLESKKNEKAALKVLQEEYRK |
| NOV67c | NFASWFAFAFPNMLVMLLFAWLWLQFVYMFSSFKKSWGCGLESKKNEKAALKVLQEEYRK |
| NOV67d | NFASWFAFAFPNMLVMLLFAWLWLQFVYMFSSFKKSWGCGLESKKNEKAALKVLQEEYRK |
| NOV67e | NFASWFAFAFPNMLVMLLFAWLWLQFVYMRFNFKKSWGCGLESKKNEKAALKVLQEEYRK |
| NOV67f | NFASWFAFAFPNMLVMLLFAWLWLQFVYMRFNFKKSWGCGLESKKNEKAALKVLQEEYRK |
| NOV67a | LGPLSFAEINVLICFFLLVILWFSRDPGFNPGWLTVAWVEGETKYVSDATVAIFVATLLF |
| NOV67b | LGPLSFAEINVLICFFLLVILWFSRDPGFMPGWLTVAWVEGETKYVSDATVAIFVATLLF |
| NOV67c | LGPLSFAEINVLICFFLLVILWFSRDPGFMPGWLTVAWVEGETKYVSDATVAIFVATLLF |
| NOV67d | LGPLSFAEINVLICFFLLVILWFSRDPGFMPGWLTVAWVEGETKSVSDATVAIFVATLLF |
| NOV67e | LGPLSFAEINVLICFFLLVILWFSRDPGFMPGWLTVAWVEGETKYVSDATVAIFVATLLF |
| NOV67f | LGPLSFAEINVLICFFLLVILWFSRDPGFMPGWLTVAWVEGETKYVSDATVAIFVATLLF |
| NOV67a | IVPSQKPKFNFRSQTEEERK-TPFYPPPLLDWKVTQEKVPWGIVLLLGGGFALAKGSEAS |
| NOV67b | IVPSQKPKFNFRSQTEEERK-TPFYPPPLLDWKVTQEKVPWGIVLLLGGGFALAKGSEAS |
| NOV67c | IVPSQKPKFNFRSQTEEGKSPVLIAPPPLLDWKVTQEKVPWGIVLLLGGGFALAKGSEAS |
| NOV67d | IVPSQKPKFNFRSQTEEGKSPVLIAPPPLLDWKVTQEKVPWGIVLLLGGGFALAKGSEAS |
| NOV67e | IVPSQKPKFNFRSQTEEERK-TPFYPPPLLDWKVTQEKVPWGIVLLLGGGFALAKGSEAS |
| NOV67f | IVPSQKPKFNFRSQTEEERK-TPFYPPPLLDWKVTQEKVPWGIVLLLGGGFALAKGSEAS |
| NOV67a | GLSVWMGKQMEPLHAVPPAAITLILSLLVAVFTECTSNVATTTLFLPIFASMSR---SIG |
| NOV67b | GLSVWMGKQMEPLHAVPPAAITLILSLLVAVFTECTSNVATTTLFLPIFASMSR---SIG |
| NOV67c | GLSVWMGKQMEPLHAVPPAAITLILSLLVAVFTECTSNVATTTLFLPIFASMSR---SIG |
| NOV67d | GLSVWMGKQMEPLHAVPPAAITLILSLLVAVFTECTSNVATTTLFLPIFASMSR---SIG |
| NOV67e | GLSVWMGKQMEPLHAVPPAAITLILSLLVAVFTECTSNVATTTLFLPIFASMVKTGVIMN |
| NOV67f | GLSVWMGKQMEPLHAVPPAAITLILSLLVAVFTECTSNVATTTLFLPIFASMN------H |
| NOV67a | LNPLYIMLPCTLSASFAFMLPVATPPNAIVFTYGHLKVADMVKTGVIMNIIGVFCVFLAV |
| NOV67b | LNPLYIMLPCTLSASFAFMLPVATPPNAIVFTYGHLKVADMVKTGVIMNIIGVFCVFLAV |
| NOV67c | LNPLYIMLPCTLSASFAFMLPVATPPNAIVFTYGHLKVADMVKTGVIMNIIGVFCVFLAV |
| NOV67d | LNPLYIMLPCTLSASFAFMLPVATPPNAIVFTYGHLKVADMVKTGVIMNIIGVFCVFLAV |
| NOV67e | IIGVFCVFLAVNTWGRAIFDLDHFPDWANVTHIET------------------------- |
| NOV67f | VPKSFCVLYGD----VAVLSFRSLAPSASIRCTSCCPVP--------------------- |
| NOV67a | NTWGRAIFDLDHFPDWANVTHIET--- |
| NOV67b | NTWGRAIFDLDHFPDWANVTHIETLEG |
| NOV67c | NTWGRAIFDLDHFPDWANVTHIET--- |
| NOV67d | NTWGRAIFDLDHFPDWANVTHIET--- |
| NOV67e | --------------------------- |
| NOV67f | --------------------------- |
| NOV67a | (SEQ ID NO: 994) |
| NOV67b | (SEQ ID NO: 996) |
| NOV67c | (SEQ ID NO: 998) |
| NOV67d | (SEQ ID NO: 1000) |
| NOV67e | (SEQ ID NO: 1002) |
| NOV67f | (SEQ ID NO: 1004) |

Further analysis of the NOV67a protein yielded the following properties shown in Table 67C.

TABLE 67C
Protein Sequence Properties NOV67a

| | |
|---|---|
| SignalP analysis: | Cleavage site between residues 39 and 40 |

PSORT II analysis:

| PSG: | a new signal peptide prediction method |
|---|---|
| | N-region: length 10; pos.chg 1; neg.chg 0 |
| | H-region: length 1; peak value 5.97 |
| | PSG score: 1.57 |
| GvH: | von Heijne's method for signal seq. recognition |
| | GvH score (threshold: −2.1): −2.36 |
| | possible cleavage site: between 30 and 31 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 11 | |
| | INTEGRAL Likelihood = −12.21 | Transmembrane 14-30 |
| | INTEGRAL Likelihood = −1.81 | Transmembrane 35-51 |
| | INTEGRAL Likelihood = −4.99 | Transmembrane 53-69 |
| | INTEGRAL Likelihood = −1.75 | Transmembrane 124-140 |
| | INTEGRAL Likelihood = −3.72 | Transmembrane 261-277 |
| | INTEGRAL Likelihood = −8.01 | Transmembrane 312-328 |
| | INTEGRAL Likelihood = −6.26 | Transmembrane 354-370 |
| | INTEGRAL Likelihood = 0.21 | Transmembrane 405-421 |
| | INTEGRAL Likelihood = −7.11 | Transmembrane 443-459 |
| | INTEGRAL Likelihood = −1.70 | Transmembrane 490-506 |
| | INTEGRAL Likelihood = −7.43 | Transmembrane 528-544 |

| | PERIPHERAL Likelihood = 0.74 (at 89) |
|---|---|
| | ALOMscore: −12.21 (number of TMSs: 11) |
| MTOP: | Prediction of membrane topology (Hartmann et al.) |
| | Center position for calculation: 21 |
| | Charge difference: −1.0 C( 2.0) - N( 3.0) |
| | N >= C: N-terminal side will be inside |
| | >>> membrane topology: type 3a |

MITDISC: discrimination of mitochondrial targeting seq

| | | | |
|---|---|---|---|
| R content: | 1 | Hyd Moment (75): | 1.18 |
| Hyd Moment (95): | 3.95 | G content: | 0 |
| D/E content: | 1 | S/T content: | 6 |
| Score: −3.21 | | | |

Gavel: prediction of cleavage sites for mitochondrial preseq

R-2 motif at 47 VRC|AY

NUCDISC: discrimination of nuclear localization signals

pat4: none
pat7: none
bipartite: none
content of basic residues: 7.7%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 94.1

COIL: Lupas's algorithm to detect coiled-coil regions

total: 0 residues

--------------------------
Final Results (k = 9/23):

66.7%: endoplasmic reticulum
22.2%: mitochondrial
11.1%: nuclear

>> prediction for CG57758-03 is end (k = 9)

A search of the NOV67a protein against the Geneseq database, a proprietary database that contains sequences published in patents and patent publication, yielded several homologous proteins shown in Table 67D.

TABLE 67D
Geneseq Results for NOV67a

| | | NOV67a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU79946 | Human transporter protein | 1 . . . 568 | 566/568 (99%) | 0.0 |
| | sequence - Homo sapiens, 568 aa. | 1 . . . 568 | 566/568 (99%) | |
| | [US2002019028-A1, 14-Feb.- | | | |
| | 2002] | | | |
| AAE21181 | Human TRICH-25 protein - Homo | 1 . . . 568 | 531/576 (92%) | 0.0 |
| | sapiens, 539 aa. [WO200212340- | 1 . . . 539 | 531/576 (92%) | |
| | A2, 14-Feb.-2002] | | | |
| ABB82951 | Human SLC13A related protein | 1 . . . 561 | 317/579 (54%) | 0.0 |
| | (GenBank Identifier No. | 1 . . . 572 | 423/579 (72%) | |
| | GI#4506979) - Homo sapiens, 592 | | | |
| | aa. [WO200298468-A1, 12-Dec.- | | | |
| | 2002] | | | |
| ABB82950 | Human SLC13A related protein | 1 . . . 561 | 317/579 (54%) | 0.0 |
| | (GenBank Identifier No. | 1 . . . 572 | 423/579 (72%) | |
| | GI#2499523) - Homo sapiens, 592 | | | |
| | aa. [WO200298468-A1, 12-Dec.- | | | |
| | 2002] | | | |
| ABB82952 | Human SLC13A related protein | 1 . . . 563 | 284/591 (48%) | e-156 |
| | (GenBank Identifier No. | 4 . . . 582 | 387/591 (65%) | |
| | GI#13653602) - Homo sapiens, | | | |
| | 602 aa. [WO200298468-A1, 12- | | | |
| | Dec.-2002] | | | |

In a BLAST search of public sequence databases, the NOV67a protein was found to have homology to the proteins shown in the BLASTP data in Table 67E.

TABLE 67E
Public BLASTP Results for NOV67a

| | | NOV67a | Identities/ | |
|---|---|---|---|---|
| Protein | | Residues/ | Similarities for | Ex- |
| Accession | | Match | the Matched | pect |
| Number | Protein/Organism/Length | Residues | Portion | Value |
| AAN86530 | Na+−coupled citrate transporter | 1 . . . 568 | 568/568 (100%) | 0.0 |
| | protein - Homo sapiens (Human), | 1 . . . 568 | 568/568 (100%) | |
| | 568 aa. | | | |
| Q8CJ44 | Sodium-coupled citrate | 1 . . . 568 | 442/572 (77%) | 0.0 |
| | transporter - Rattus norvegicus | 1 . . . 572 | 502/572 (87%) | |
| | (Rat), 572 aa. | | | |
| AAH44437 | Similar to solute carrier family | 5 . . . 564 | 331/598 (55%) | 0.0 |
| | 13, member 2 - Brachydanio rerio | 10 . . . 605 | 437/598 (72%) | |
| | (Zebrafish) (Danio rerio), 613 aa. | | | |
| AA027449 | Sodium dicarboxylate co- | 11 . . . 563 | 318/582 (54%) | 0.0 |
| | transporter - Didelphis | 11 . . . 587 | 422/582 (71%) | |
| | marsupialis virginiana (North | | | |
| | American opossum), 605 aa. | | | |
| Q13183 | Solute carrier family 13, member | 1 . . . 561 | 317/579 (54%) | 0.0 |
| | 2 (Renal sodium/dicarboxylate | 1 . . . 572 | 423/579 (72%) | |
| | cotransporter) | | | |
| | (Na(+)/dicarboxylate | | | |
| | cotransporter 1) (NaDC-1) - | | | |
| | Homo sapiens (Human), 592 aa. | | | |

PFam analysis indicates that the NOV67a protein contains the domains shown in the Table 67F.

TABLE 67F
Domain Analysis of NOV67a

| | | Identities/ | |
|---|---|---|---|
| | NOV67a | Similarities | |
| | Match | for the | |
| Pfam Domain | Region | Matched Region | Expect Value |
| DcuC | 30 . . . 548 | 88/593 (15%) | 0.73 |
| | | 324/593 (55%) | |
| Na_sulph_symp | 6 . . . 554 | 159/603 (26%) | 2e-143 |
| | | 423/603 (70%) | |

### Example 68

The NOV68 clone was analyzed, and the nucleotide and encoded polypeptide sequences are shown in Table 68A.

TABLE 68A
NOV68 Sequence Analysis

| | | |
|---|---|---|
| NOV68a, CG58504-01 | SEQ ID NO: 1005 | 5115 bp |

| | | |
|---|---|---|
| DNA Sequence | ORF Start: ATG at 327 | ORF Stop: TAA at 5106 |

GAATTCCGGGAGCGGGCGGGCTGCGAGGCCGCGGGGCATGCGGGAGGCGGAGGGGTGGGACCGGGTGG
CTGCGCCCATTCCACACCCGCCGAAAGCGGACACTGTCAGCTGAATCACTCCCCTTTTAGGAGGAGGG
AGGGGGAAAAGGTGTCTAGCTAATTTCTGCTTAAAAAAGCACAGGAGATCGCGGGTCAGCTTTGCAGT
CGCTGCCTTCTCGCGCCTGACCATGCACCCCTGCATCTTCCTGCTGGGCACAGGCGAGCGCTTTATTT
CTGGAGCTGAGGGCTAAAACTTTTTTCACTTTTCTTCTCCTCAACATCTGAATCATGCCATGTGCCCA
GAGGAGCTGGCTTGCAAACCTTTCCGTGGTGGCTCAGCTCCTTAACTTTGGGGCGCTTTGCTATGGGA
GACAGCCTCAGCCAGGCCCGGTTCGCTTCCCGGACAGGAGGCAAGAGCATTTTATCAAGGGCCTGCCA
GAATACCACGTGGTGGGTCCAGTCCGAGTAGATGCCAGTGGGCATTTTTTGTCATATGGCTTGCACTA
TCCCATCACGAGCAGCAGGAGGAAGAGAGATTTGGATGGCTCAGAGGACTGGGTGTACTACAGAATTT
CTCACGAGGAGAAGGACCTGTTTTTTAACTTGACGGTCAATCAAGGATTTCTTTCCAATAGCTACATC
ATGGAGAAGAGATATGGGAACCTCTCCCATGTTAAGATGATGGCTTCCTCTGCCCCCCTCTGCCATCT
CAGTGGCACGGTTCTACAGCAGGGCACCAGAGTTGGGACGGCAGCCCTCAGTGCCTGCCATGGACTGA
CTGGATTTTTCCAACTACCACATGGAGACTTTTTCATTGAACCCGTGAAGAAGCATCCACTGGTTGAG
GGAGGGTACCACCCGCACATCGTTTACAGGAGGCAGAAAGTTCCAGAAACCAAGGAGCCAACCTGTGG
ATTAAAGGACAGTGTTAACATCTCCCAGAAGCAAGAGCTATGGCGGGAGAAGTGGGAGAGGCACAACT
TGCCAAGCAGAAGCCTCTCTCGGCGTTCCATCAGCAAGGAGAGATGGGTGGAGACACTGGTGGTGGCC
GACACAAAGATGATTGAATACCATGGGAGTGAGAATGTGGAGTCCTACATCCTCACCATCATGAACAT
GGTCACTGGGTTGTTCCATAACCCAAGCATTGGCAATGCAATTCACATTGTTGTGGTTCGGCTCATTC
TACTCGAAGAAGAAGAGCAAGGACTGAAAATAGTTCACCATGCAGAAAAGACACTGTCTAGCTTCTGC
AAGTGGCAGAAGAGTATCAATCCCAAGAGTGACCTCAATCCTGTTCATCACGACGTGGCTGTCCTTCT
CACCAGAAAGGACATCTGTGCTGGTTTCAATCGCCCCTGCGAGACCCTGGGCCTGTCTCACCTTTCAG
GAATGTGTCAGCCTCACCGCAGTTGTAACATCAATGAAGATTCGGGACTCCCTCTGGCTTTCACAATT
GCCCATGAGCTAGGACACAGCTTCGGCATCCAGCATGATGGGAAAGAAAATGACTGTGAGCCTGTGGG
CAGACATCCGTACATCATGTCCCGCCAGCTCCAGTACGATCCCACTCCGCTGACATGGTCCAAGTGCA
GCGAGGAGTACATCACCCGCTTCTTGGACCGAGGCTGGGGGTTCTGTCTTGATGACATACCTAAAAAG
AAAGGCTTGAAGTCCAAGGTCATTGCCCCCGGAGTGATCTATGATGTTCACCACCAGTGCCAGCTACA
ATATGGACCCAATGCTACCTTCTGCCAGGAAGTAGAAAACGTCTGCCAGACACTGTGGTGCTCCGTGA
AGGGCTTTTGTCGCTCTAAGCTGGACGCTGCTGCAGATGGAACTCAATGTGGTGAGAAGAAGTGGTGT
ATGGCAGGCAAGTGCATCACAGTGGGGAAGAAACCAGAGAGCATTCCTGGAGGCTGGGGCCGCTGGTC
ACCCTGGTCCCACTGTTCCAGGACCTGTGGGGCTGGAGTCCAGAGCGCAGAGAGGCTCTGCAACAACC
CCGAGCCAAAGTTTGGAGGGAAATATTGCACTGGAGAAAGAAAACGCTATCGCTTGTGCAACGTCCAC
CCCTGTCGCTCAGAGGCACCAACATTTCGGCAGATGCAGTGCAGTGAATTTGACACTGTTCCCTACAA
GAATGAACTCTACCACTGGTTTCCCATTTTTAACCCAGCACATCCTTGTGAGCTCTACTGCCGACCCA
TAGATGGCCAGTTTTCTGAGAAAATGCTGGATGCTGTCATTGATGGTACCCCTTGCTTTGAAGGCGGC
AACAGCAGAAATGTCTGTATTAATGGCATATGTAAGATGGTTGGCTGTGACTATGAGATCGATTCCAA
TGCCACCGAGGATCGCTGCGGTGTGTGCCTGGGAGATGGCTCTTCCTGCCAGACTGTGAGAAAGATGT
TTAAGCAGAAGGAAGGATCTGGTTATGTTGACATTGGGCTCATTCCAAAAGGAGCAAGGGACATAAGA
GTGATGGAAATTGAGGGAGCTGGAAACTTCCTGGCCATCAGGAGTGAAGATCCTGAAAAATATTACCT
GAATGGAGGGTTTATTATCCAGTGGAACGGGAACTATAAGCTGGCAGGGACTGTCTTTCAGTATGACA
GGAAAGGAGACCTGGAAAAGCTGATGGCCACAGGTCCCACCAATGAGTCTGTGTGGATCCAGCTTCTA
TTCCAGGTGACTAACCCTGGCATCAAGTATGAGTACACAATCCAGAAAGATGGCCTTGACAATGATGT
TGAGCAGATGTACTTCTGGCAGTACGGCCACTGGACAGAGTGCAGTGTGACCTGCGGGACAGGTATCC
GCCGCCAAACTGCCCATTGCATAAAGAAGGGCCGCGGGATGGTGAAAGCTACATTCTGTGACCCAGAA
ACACAGCCCAATGGGAGACAGAAGAAGTGCCATGAAAAGGCTTGTCCACCCAGGTGGTGGGCAGGGGA
GTGGGAAGCATGCTCGGCGACATGCGGGCCCCACGGGGAGAAGAAGCGAACCGTGCTGTGCATCCAGA
CCATGGTCTCTGACGAGCAGGCTCTCCCGCCCACAGACTGCCAGCACCTGCTGAAGCCCAAGACCCTC
CTTTCCTGCAACAGAGACATCCTGTGCCCCTCGGACTGGACAGTGGGCAACTGGAGTGAGTGTTCTGT
TTCCTGTGGTGGTGGAGTGCGGATTCGCAGTGTCACATGTGCCAAGAACCATGATGAACCTTGCGATG
TGACAAGGAAACCCAACAGCCGAGCTCTGTGTGGCCTCCAGCAATGCCCTTCTAGCCGGAGAGTTCTG
AAACCAAACAAAGGCACTATTTCCAATGGAAAAAACCCACCAACACTAAAGCCCGTCCCTCCACCTAC
ATCCAGGCCCAGAATGCTGACCACACCCACAGGGCCTGAGTCTATGAGCACAAGCACTCCAGCAATCA
GCAGCCCTAGTCCTACCACAGCCTCCAAAGAAGGAdACCTGGGTGGGAAACAGTGGCAAGATAGCTCA
ACCCAACCTGAGCTGAGCTCTCGCTATCTCATTTCCACTGGAAGCACTTCCCAGCCCATCCTCACTTC
CCAATCCTTGAGCATTCAGCCAAGTGAGGAAAATGTTTCCAGTTCAGATACTGGTCCTACCTCGGAGG
GAGGCCTTGTAGCTACAACAACAAGTGGTTCTGGCTTGTCATCTTCCCGCAACCCTATCACTTGGCCT
GTGACTCCATTTTACAATACCTTGACCAAAGGTCCAGAAATGGAGATTCACAGTGGCTCAGGGGAAGA
AAGAGAACAGCCTGAGGACAAAGATGAAAGCAATCCTGTAATATGGACCAAGATCAGAGTACCTGGAA
ATGACGCTCCAGTGGAAAGTACAGAAATGCCACTTGCACCTCCACTAACACCAGATCTCAGCAGGGAG
TCCTGGTGGCCACCCTTCAGCACAGTAATGGAAGGACTGCTCCCCAGCCAAAGGCCCACTACTTCCGA
AACTGGGACACCCAGAGTTGAGGGGATGGTTACTGAAAAGCCAGCCAACACTCTGCTCCCTCTGGGAG
GAGACCACCAGCCAGAACCCTCAGGAAAGACGGCAAACCGTAACCACCTGAAACTTCCAAACAACATG
AACCAAACAAAAAGTTCTGAACCAGTCCTGACTGAGGAGGATGCAACAAGTCTGATTACTGAGGGCTT
TTTGCTAAATGCCTCCAATTACAAGCAGCTCACAAACGGCCACGGCTCTGCACACTGGATCGTCGGAA
ACTGGAGCGAGTGCTCCACCACATGTGGCCTGGGGGCCTACTGGAAAAGGGTGGAGTGCACCACCCAG
ATGGATTCTGACTGTGCGGCCATCCAGAGACCTGACCCTGCAAAAAGATGCCACCTCCGTCCCTGTGC
TGGCTGGAAAGTGGGAAACTGGAGCAAGTGCTCCAGAAACTGCAGTGGGGGCTTCAAGATACGCGAGA
TTCAGTGCGTGGACAGCCGGGACCACCGGAACCTGAGGCCATTTCACTGCCAGTTCCTGGCCGGCATT
CCTCCCCCATTGAGCATGAGCTGTAACCCGGAGCCCTGTGAGGCGTGGCAGGTGGAGCCTTGGAGCCA
GTGCTCCAGGTCCTGTGGAGGTGGAGTTCAGGAGAGAGGAGTGTTCTGTCCAGGAGGCCTCTGTGATT
GGACAAAAAGACCCACATCCACCATGTCTTGCAATGAGCACCTGTGCTGTCACTGGGCCACTGGGAAC
TGGGACCTGTGTTCCACTTCCTGTGGAGGTGGCTTTCAGAAGAGGATTGTCCAATGTGTGCCCTCAGA
GGGCAATAAAACTGAAGACCAAGACCAATGTCTATGTGATCACAAACCCAGACCTCCAGAATTCAAAA
AATGCAACCAGCAGGCCTGCAAGAAAAGTGCCGATTTACTTTGCACTAAGGACAAACTGTCAGCCAGT
TTCTGCCAGACACTGAAAGCCATGAAGAAATGTTCTGTGCCCACCGTGAGGGCTGAGTGCTGCTTCTC
GTGTCCCCAGACACACATCACACACACCCAAAGGCAAAGAAGGCAACGGTTGCTCCAAAAGTCAAAAG
AACTCTAAGCCCAAA

NOV68a, CG58504-01

| | | | |
|---|---|---|---|
| Protein Sequence | SEQ ID NO: 1006 | 1593 aa | MW at 177543.9kD |

MPCAQRSWLANLSVVAQLLNFGALCYGRQPQPGPVRFPDRRQEHFIKGLPEYHVVGPVRVDASGHFLS
YGLHYPITSSRRKRDLDGSEDWWIYRISHEEKDLFFNLTVNQGFLSNSYIMEKRYGNLSHVKMMASSA
PLCHLSGTVLQQGTRVGTAALSACHGLTGFFQLPHGDFFIEPVKKHPLVEGGYHPHIVYRRQKVPETK
EPTCGLKDSVNISQKQELWREKWERHNLPSRSLSRRSISKERWVETLVVADTKMIEYHGSENVESYIL
TIMNMVTGLFHNPSIGNAIHIVVVRLILLEEEEQGLKIVHHAEKTLSSFCKWQKSINPKSDLNPVHHD
VAVLLTRKDICAGFNRPCETLGLSHLSGMCQPHRSCNINEDSGLPLAFTIAHELGHSFGIQHDGKEND
CEPVGRHPYIMSRQLQYDPTPLTWSKCSEEYITRFLDRGWGFCLDDIPKKKGLKSKVIAPGVIYDVHH
QCQLQYGPNATFCQEVENVCQTLWCSVKGFCRSKLDAAADGTQCGEKKWCMAGKCITVGKKPESIPGG
WGRWSPWSHCSRTCGAGVQSAERLCNNPEPKFGGKYCTGERKRYRLCNVHPCRSEAPTFRQMQCSEFD
TVPYKNELYHWFPIFNPAHPCELYCRPIDGQFSEKMLDAVIDGTPCFEGGNSRNVCINGICKMVGCDY
EIDSNATEDRCGVCLGDGSSCQTVRKMFKQKEGSGYVDIGLIPKGARDIRVMEIEGAGNFLAIRSEDP
EKYYLNGGFIIQWNGNYKLAGTVFQYDRKGDLEKLMATGPTNESVWIQLLFQVTNPGIKYEYTIQKDG
LDNDVEQMYFWQYGHWTECSVTCGTGIRRQTAHCIKKGRGMVKATFCDPETQPNGRQKKCHEKACPPR
WWAGEWEACSATCGPHGEKKRTVLCIQTMVSDEQALPPTDCQHLLKPKTLLSCNRDILCPSDWTVGNW
SECSVSCGGGVRIRSVTCAKNHDEPCDVTRKPNSRALCGLQQCPSSRRVLKPNKGTISNGKNPPTLKP
VPPPTSRPRMLTTPTGPESMSTSTPAISSPSPTTASKEGDLGGKQWQDSSTQPELSSRYLISTGSTSQ
PILTSQSLSIQPSEENVSSSDTGPTSEGGLVATTTSGSGLSSSRNPITWPVTPFYNTLTKGPEMEIHS
GSGEEREQPEDKDESNPVIWTKIRVPGNDAPVESTEMPLAPPLTPDLSRESWWPPFSTVMEGLLPSQR
PTTSETGTPRVEGMVTEKPANTLLPLGGDHQPEPSGKTANRNHLKLPNNMNQTKSSEPVLTEEDATSL
ITEGFLLNASNYKQLTNGHGSAHWIVGNWSECTTCGLAGAYWKRVECTTQMDSDCAAIQRPDPAKRCH
LRPCAGWKVGNWSKCSRNCSGGFKIREIQCVDSRDHRNLRPFHCQFLAGIPPPLSMSCNPEPCEAWQV
EPWSQCSRSCGGGVQERGVFCPGGLCDWTKRPTSTMSCNEHLCCHWATGNWDLCSTSCGGGFQKRIVQ
CVPSEGNKTEDQDQCLCDHKPRPPEFKKCNQQACKKSADLLCTKDKLSASFCQTLKAMKKCSVPTVRA
ECCFSCPQTHITHTQRQRRQRLLQKSKEL

| | | |
|---|---|---|
| NOV68b, 169648376 | SEQ ID NO: 1007 | 1068 bp |

| | | |
|---|---|---|
| DNA Sequence | ORF Start: at 1 | ORF Stop: end of sequence |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCTCAGTGGCACGGTTCTACAGC
AGGGCACCAGAGTTGGGATGGCAGCCCTCAGTGCCTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAACTTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTGCATCCTCACCATCATGAACATGGTCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68b, 169648376

| | | | |
|---|---|---|---|
| Protein Sequence | SEQ ID NO: 1008 | 356 aa | MW at 40336.9kD |

KLNLTVNQGFLSNSYIMEKRYGNLSHVKMMASSAPLCHLSGTVLQQGTRVGMAALSACHGLTGFFQLP
HGDFFIEPVKKHPLVEGGYHPHIVYRRQKVPETKEPTCGLKDSVNISQKQELWREKWERHNLPSRSLS
RRSISKERWVETLVVADTKMIEYHGSENVESCILTIMNMVTGLFHNPSIGNAIHIVVVRLILLEEEEQ
GLKIVHHAEKTLSSFCKWQKSINPKSDLNPVHHDVAVLLTRKDICAGFNRPCETLGLSHLSGMCQPHR
SCNINEDSGLPLAFTIAHELGHSFGIQHDGKENDCEPVGRHPYIMSRQLQYDPTPLTWSKCSEEYITR
FLDRGWGFCLDDIPLE

| | | |
|---|---|---|
| NOV68c, 169648388 | SEQ ID NO: 1009 | 1068 bp |

| | | |
|---|---|---|
| DNA Sequence | ORF Start: at 1 | ORF Stop: end of sequence |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCTCAGTGGCACGGTTCTACAGC
AGGGCACCAGAGTTGGGACGGCAGCCCTCAGTGCCTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAACTTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTACATCCTCACCATCATGAACATGATCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68c, 169648388

| | | | |
|---|---|---|---|
| Protein Sequence | SEQ ID NO: 1010 | 356 aa | MW at 40380.8kD |

KLNLTVNQGFLSNSYIMEKRYGNLSHVKMMASSAPLCHLSGTVLQQGTRVGTAALSACHGLTGFFQLP
HGDFFIEPVKKHPLVEGGYHPHIVYRRQKVPETKEPTCGLKDSVNISQKQELWREKWERHNLPSRSLS
RRSISKERWVETLVVADTKMIEYHGSENVESYILTIMNMITGLFHNPSIGNAIHIVVVRLILLEEEEQ
GLKIVHHAEKTLSSFCKWQKSINPKSDLNPVHHDVAVLLTRKDICAGFNRPCETLGLSHLSGMCQPHR
SCNINEDSGLPLAFTIAHELGHSFGIQHDGKENDCEPVGRHPYIMSRQLQYDPTPLTWSKCSEEYITR
FLDRGWGFCLDDIPLE

| | | |
|---|---|---|
| NOV68d, 169648365 | SEQ ID NO: 1011 | 1068 bp |

| | | |
|---|---|---|
| DNA Sequence | ORF Start: at 1 | ORF Stop: end of sequence |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCICAGTGGCACGGTTCTACAGC
AGGGCACCAGAGTTGGGACGGCAGCCCTCAGTGCCTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAAATTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTACATCCTCACCATCATGAACATGGTCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68d, 169648365

| | | | |
|---|---|---|---|
| Protein Sequence | SEQ ID NO: 1012 | 356 aa | MW at 40366.8kD |

KLNLTVNQGFLSNSYIMEKRYGNLSHVKMMASSAPLCHLSGTVLQQGTRVGTAALSACHGLTGFFQLP
HGDFFIEPVKKHPLVEGGYHPHIVYRRQKVPETKEPTCGLKDSVNISQKQELWREKWERHNLPSRSLS
RRSISKERWVETLVVADTKMIEYHGSENVESYILTINNMVTGLFHNPSIGNAIHIVVVRLILLEEEEQ
GLKIVHHAEKTLSSFCKWQKSINPKSDLNPVHHDVAVLLTRKDICAGFNRPCETLGLSHLSGMCQPHR
SCNINEDSGLPLAFTIAHELGHSFGIQHDGKENDCEPVGRHPYIMSRQLQYDPTPLTWSKCSEEYITR
FLDRGWGFCLDDIPLE

| | | |
|---|---|---|
| NOV68e, 284068250 | SEQ ID NO: 1013 | 4777 bp |

| | | |
|---|---|---|
| DNA Sequence | ORF Start: at 1 | ORF Stop: TGA at 4729 |

AGATCTTATGGGAGACAGCCTCAGCCAGGCCCGGTTCGCTTCCCGGACAGGAGGCAAGAGCATTTTAT
CAAGGGCCTGCCAGAATACCACGTGGTGGGTCCAGTCCGAGTAGATGCCAGTGGGCATTTTTTGTCAT
ATGGCTTGCACTATCCCATCACGAGCAGCAGGAGGAAGAGAGATTTGGATGGCTCAGAGGACTGGGTG
TACTACAGAATTTCTCACGAGGAGAAGGACCTGTTTTTTAACTTGACGGTCAATCAAGGATTTCTTTC
CAATAGCTACATCATGGAGAAGAGATATGGGAACCTCTCCCATGTTAAGATGATGGCTTCTTCTGCCC
CCCTCTGCCATCTCAATGGCACGGTTCTACAGCAGGGCACCAGAGTTGGGACGGCAGCCCTCAGTGCC
TGCCATGGACTGACTGGATTTTTCCAACTACCACATGGAGACTTTTTCATTGAACCCGTGAAGAAGCA
TCCACTGGTTGAGGGAGGGTACCACCCGCACATCGTTTACAGGAGGCAGAAAGTTCCAGAAACCAAGG
AGCCAACCTGTGGATTAAAGGACAGTGTTAACATCTCCCAGAAGCAAGAGCTATGGCGGGAGAAGTGG
GAGAGGCACAACTTGCCAAGCAGAAGCCTCTCTCGGCGTTCCATCAGCAAGGAGAGATGGGTGGAGAC
ACTGGTGGTGGCCGACACAAAGATGATTGAATACCATGGGAGTGAGAATGTGGAGTCCTACATCCTCA
CCATCATGAACATGGTCACTGGGTTGTTCCATAACCCAAGCATTGGCAATGCAATTCACATTGTTGTG
GTTCGGCTCATTCTACTCGAAGAAGAAGAGCAAGGACTGAAAATAGTTCACCATGCAGAAAAGACACT
GTCTAGCTTCTGCAAGTGGCAGAAGAGTATCAATCCCAAGAGTGACCTCAATCCTGTTCATCACGACG
TGGCTGTCCTTCTCACCAGAAAGGACATCTGTGCTGGTTTCAATCGCCCGTGCGAGACCCTGGGCCTG
TCTCACCTTTCAGGAATGTGTCAGCCTCACCGCAGTTGTAACATCAATGAAGATTCGGGACTCCCTCT
GGCTTTCACAATTGCCCATGAGCTAGGACACAGCTTCGGCATCCAGCATGATGGGAAAGAAAATGACT
GTGAGCCTGTGGGCAGACATCCGTACATCATGTCCCGCCAGCTCCAGTACGATCCCACTCCGCTGACA
TGGTCCAAGTGCAGCGAGGAGTACATCACCCGCTTCTTGGACCGAGGCTGGGGGTTCTGTCTTGATGA
CATACCTAAAAAGAAAGGCTTGAAGTCCAAGGTCATTGCCCCCGGAGTGATCTATGATGTTCACCACC
AGTGCCAGCTACAATATGGACCCAATGCTACCCTCTGCCAGGAAGTAGAAAACGTCTGCCAGACACTG
TGGTGCTCCGTGAAGGGCTTTTGTCGCTCTAAGCTGGACGCTGCTGCAGATGGAACTCAATGTGGTGA
GAAGAAGTGGTGTATGGCAGGCAAGTGCATCACAGTGGGGAAGAAACCAGAGAGCATTCCTGGAGGCT
GGGGCCGCTGGTCACCCTGGTCCCACTGTTCCAGGACCTGTGGGGCTGGAGTCCAGAGCGCAGAGAGG
CTCTGCAACAACCCCGAGCCAAAGTTTGGAGGGAAATATTGCACTGGAGAAAGAAAACGCTATCGCTT
GTGCAACGTCCACCCCTGTCGCTCAGAGGCACCAACATTTCGGCAGATGCAGTGCAGTGAATTTGACA
CTGTTCCCTACAAGAATGAACTCTACCACTGGTTTCCCATTTTTAACCCAGCACATCCTTGTGAGCTC
TACTGCCGACCCATAGATGGCCAGTTTTCTGAGAAAATGCTGGATGCTGTCATTGATGGTACCCCTTG
CTTTGAAGGCGGCAACAGCAGAAATGTCTGTATTAATGGCATATGTAAGATGGTTGGCTGTGACTATG
AGATCGATTCCAATGCCACCGAGGATCGCTGCGGTGTGTGCCTGGGAGATGGCTCTTCCTGCCAGACT
GTGAGAAAGATGTTTAAGCAGAAGGAAGGATCTGGTTATGTTGACATTGGGCTCATTCCAAAAGGAGC
AAGGGACATAAGAGTGATGGAAATTGAGGGAGCTGGAAACTTCCTGGCCATCAGGAGTGAAGATCCTG
AAAAATATTACCTGAATGGAGGGTTTATTATCCAGTGGAACGGGAACTATAAGCTGGCAGGGACTGTC
TTTCAGTATGACAGGAAAGGAGACCTGGAAAAGCTGATGGCCACAGGTCCCACCAATGAGTCTGTGTG
GATCCAGCTTCTATTCCAGGTGACTAACCCTGGCATCAAGTATGAGTACACAATCCAGAAAGATGGCC
TTGACAATGATGTTGAGCAGCAGATGTACTTCTGGCAGTACGGCCACTGGACAGAGTGCAGTGTGACC
TGCGGGACAGGTATCCGCCGCCAAACTGCCCATTGCATAAAGAAGGGCCGCGGGATGGTGAAAGCTAC
ATTCTGTGACCCAGAAACACAGCCCAATGGGAGACAGAAGAAGTGCCATGAAAAGGCTTGTCCACCCA
GGTGGTGGGCAGGGGAGTGGGAAGCATGCTCGGCGACATGCGGGCCCCACGGGGAGAAGAAGCGAACC
GTGCTGTGCATCCAGACCATGGTCTCTGACGAGCAGGCTCTCCCGCCCACAGACTGCCAGCACCTGCT
GAAGCCCAAGACCCTCCTTTCCTGCAACAGAGACATCCTGTGCCCCTCGGACTGGACAGTGGGCAACT
GGAGTGAGTGTTCTGTTTCCTGTGGTGGTGGAGTGCGGATTCGCAGTGTCACATGTGCCAAGAACCAT
GATGAACCTTGCGATGTGACAAGGAAACCCAACAGCCGAGCTCTGTGTGGCCTCCAGCAATGCCCTTC
TAGCCGGAGAGTTCTGAAACCAAACAAAGGCACTATTTCCAATGGAAAAAACCCACCAACACTAAAGC
CCGTCCCTCCACCTACATCCAGGCCCAGAATGCTGACCACACCCACAGGGCCTGAGTCTATGAGCACA
AGCACTCCAGCAATCAGCAGCCCTAGTCCTACCACAGCCTCCAAAGAAGGAGACCTGGGTGGGAAACA
GTGGCAAGATAGCTCAACCCAACCTGAGCTGAGCTCTCGCTATCTCATTTCCACTGGAAGCACTTCCC
AGCCCATCCTCACTTCCCAATCCTTGAGCATTCAGCCAAGTGAGGAAAATGTTTCCAGTTCAGATACT
GGTCCTACCTCGGAGGGAGGCCTTGTAGCTACAACAACAAGTGGTTCTGGCTTGTCATCTTCCCGCAA
CCCTATCACTTGGCCTGTGACTCCATTTTACAATACCTTGACCAAAGGTCCAGAAATGGAGATTCACA
GTGGCTCAGGGGAAGAAAGAGAACAGCCTGAGGACAAAGATGAAAGCAATCCTGTAATATGGACCAAG
ATCAGAGTACCTGGAAATGACGCTCCAGTGGAAAGTACAGAAATGCCACTTGCACCTCCACTAACACC
AGATCTCAGCAGGGAGTCCTGGTGGCCACCCTTCAGCACAGTAATGGAAGGACTGCTCCCCAGCCAAA
GGCCCACTACTTCCGAAACTGGGACACCCAGAGTTGAGGGGATGGTTACTGAAAAGCCAGCCAACACT
CTGCTCCCTCTGGGAGGAGACCACCAGCCAGAACCCTCAGGAAAGACGGCAAACCGTAACCACCTGAA
ACTTCCAAACAACATGAACCAAACAAAAAGTTCTGAACCAGTCCTGACTGAGGAGGATGCAACAAGTC
TGATTACTGAGGGCTTTTTGCTAAATGCCTCCAATTACAAGCAGCTCACAAACGGCCACGGCTCTGCA
CACTGGATCGTCGGAAACTGGAGCGAGTGCTCCACCACATGTGGCCTGGGGGCCTACTGGAGAAGGGT
GGAGTGCAGCACCCAGATGGATTCTGACTGTGCGGCCATCCAGAGACCTGACCCTGCAAAAAGATGCC
ACCTCCGTCCCTGTGCTGGCTGGAAAGTGGGAAACTGGAGCAAGTGCTCCAGAAACTGCAGTGGGGGC
TTCAAGATACGCGAGATTCAGTGCGTGGACAGCCGGGACCACCGGAACCTGAGGCCATTTCACTGCCA
GTTCCTGGCCGGCATTCGTCCCCCATTGAGCATGAGCTGTAACCCGGAGCCCTGTGAGGCGTGGCAGG
TGGAGCCTTGGAGCCAGTGCTCCAGGTCCTGTGGAGGTGGAGTTCAGGAGAGAGGAGTGTTCTGTCCA
GGAGGCCTCTGTGATTGGACAAAAAGACCCACATCCACCATGTCTTGCAATGAGCACCTGTGCTGTCA
CTGGGCCACTGGGAACTGGGACCTGTGTTCCACTTCCTGTGGAGGTGGCTTTCAGAAGAGGACTGTCC
AATGTGTGCCCTCAGAGGGCAATAAAACTGAAGACCAAGACCAATGTCTATGTGATCACAAACCCAGA
CCTCCAGAATTCAAAAAATGCAACCAGCAGGCCTGCAAGAAAAGTGCCGATTTACTTTGCACTAAGGA
CAAACTGTCAGCCAGTTTCTGCCAGACACTGAAAGCCATGAAGAAATGTTCTGTGCCCACCGTGAGGG
CTGAGTGCTGCTTCTCGTGTCCCCAGACACACATCACACACACCCAAAGGCAAAGAAGGCAACGGTTG
CTCCAAAAGTCAAAAGAACTCCTCGAGGAGTTCTTTTGACTTTTGGAGCAACCGGGCCTGGCTGAGGC
TGTCTCCCATAAGATCT

NOV68e, 284068250

| | | | |
|---|---|---|---|
| Protein Sequence | SEQ ID NO: 1014 | 1576 aa | MW at 175887.8kD |

RSYGRQPQPGPVRFPDRRQEHFIKGLPEYHVVGPVRVDASGHFLSYGLHYPITSSRRKRDLDGSEDWV
YYRISHEEKDLFFNLTVNQGFLSNSYIMEKRYGNLSHVKNMASSAPLCHLNGTVLQQGTRVGTAALSA
CHGLTGFFQLPHGDFFIEPVKKHPLVEGGYHPHIVYRRQKVPETKEPTCGLKDSVNISQKQELWREKW
ERHNLPSRSLSRRSISKERWVETLVVADTKMIEYHGSENVESYILTINNMVTGLFHNPSIGNAIHIVV
VRLILLEEEEQGLKIVHHAEKTLSSFCKWQKSINPKSDLNPVHHDVAVLLTRKDICAGFNRPCETLGL
SHLSGMCQPHRSCNINEDSGLPLAFTIAHELGHSFGIQHDGKENDCEPVGRHPYIMSRQLQYDPTPLT
WSKCSEEYITRFLDRGWGFCLDDIPKKKGLKSKVIAPGVIYDVHHQCQLQYGPNATLCQEVENVCQTL
WCSVKGFCRSKLDAAADGTQCGEKKWCMAGKCITVGKKPESIPGGWGRWSPWSHCSRTCGAGVQSAER
LCNNPEPKFGGKYCTGERKRYRLCNVHPCRSEAPTFRQMQCSEFDTVPYKNELYHWFPIFNPAHPCEL
YCRPIDGQFSEKMLDAVIDGTPCFEGGNSRNVCINGICKMVGCDYEIDSNATEDRCGVCLGDGSSCQT
VRKMFKQKEGSGYVDIGLIPKGARDIRVMEIEGAGNFLAIRSEDPEKYYLNGGFIIQWNGNYKLAGTV
FQYDRKGDLEKLMATGPTNESVWIQLLFQVTNPGIKYEYTIQKDGLDNDVEQQMYFWQYGHWTECSVT
CGTGIRRQTAHCIKKGRGMVKATFCDPETQPNGRQKKCHEKACPPRWWAGEWEACSATCGPHGEKKRT
VLCIQTMVSDEQALPPTDCQHLLKPKTLLSCNRDILCPSDWTVGNWSECSVSCGGGVRIRSVTCAKNH
DEPCDVTRKPNSRALCGLQQCPSSRRVLKPNKGTISNGKNPPTLKPVPPPTSRPRMLTTPTGPESMST
STPAISSPSPTTASKEGDLGGKQWQDSSTQPELSSRYLISTGSTSQPILTSQSLSIQPSEENVSSSDT
GPTSEGGLVATTTSGSGLSSSRNPITWPVTPFYNTLTKGPEMEIHSGSGEEREQPEDKDESNPVIWTK
IRVPGNDAPVESTEMPLAPPLTPDLSRESWWPPFSTVMEGLLPSQRPTTSETGTPRVEGMVTEKPANT
LLPLGGDHQPEPSGKTANRNHLKLPNNMNQTKSSEPVLTEEDATSLITEGFLLNASNYKQLTNGHGSA
HWIVGNWSECSTTCGLGAYWRRVECSTQMDSDCAAIQRPDPAKRCHLRPCAGWKVGNWSKCSRNCSGG
FKIREIQCVDSRDHRNLRPFHCQFLAGIPPPLSMSCNPEPCEAWQVEPWSQCSRSCGGGVQERGVFCP
GGLCDWTKRPTSTMSCNEHLCCHWATGNWDLCSTSCGGGFQKRTVQCVPSEGNKTEDQDQCLCDHKPR
PPEFKKCNQQACKKSADLLCTKDKLSASFCQTLKAMKKCSVPTVRAECCFSCPQTHITHTQRQRRQRL
LQKSKELLEEFF

| | | |
|---|---|---|
| NOV68f, 305867866 | SEQ ID NO: 1015 | 4777 bp |

| | | |
|---|---|---|
| DNA Sequence | ORF Start: at 1 | ORF Stop: TGA at 4729 |

AGATCTTATGGGAGACAGCCTCAGCCAGGCCCGGTTCGCTTCCCGGACAGGAGGCAAGAGCATTTTAT
CAAGGGCCTGCCAGAATACCACGTGGTGGGTCCAGTCCGAGTAGATGCCAGTGGGCATTTTTTGTCAT
ATGGCTTGCACTATCCCATCACGAGCAGCAGGAGGAAGAGAGATTTGGATGGCTCAGAGGACTGGGTG
TACTACAGAATTTCTCACGAGGAGAAGGACCTGTTTTTTAACTTGACGGTCAATCAAGGATTTCTTTC
CAATAGCTACATCATGGAGAAGAGATATGGGAACCTCTCCCATGTTAAGATGATGGCTTCCTCTGCCC
CCCTCTGCCATCTCAGTGGCACGGTTCTACAGCAGGGCACCAGAGTTGGGACGGCAGCCCTCAGTGCC
TGCCATGGACTGACTGGATTTTTCCAACTACCACATGGAGACTTTTTCATTGAACCCGTGAAGAAGCA
TCCACTGGTTGAGGGAGGGTACCACCCGCACATCGTTTACAGGAGGCAGAAAGTTCCAGAAACCAAGG
AGCCAACCTGTGGATTAAAGGACAGTGTTAACATCTCCCAGAAGCAAGAGCTATGGCGGGAGAAGTGG
GAGAGGCACAACTTGCCAAGCAGAAGCCTCTCTCGGCGTTCCATCAGCAAGGAGAGATGGGTGGAGAC
ACTGGTGGTGGCCGACACAAAGATGATTGAATACCATGGGAGTGAGAATGTGGAGTCCTACATCCTCA
CCATCATGAACATGGTCACTGGGTTGTTCCATAACCCAAGCATTGGCAATGCAATTCACATTGTTGTG
GTTCGGCTCATTCTACTCGAAGAAGAAGAGCAAGGACTGAAAATAGTTCACCATGCAGAAAAGACACT
GTCTAGCTTCTGCAAGTGGCAGAAGAGTATCAATCCCAAGAGTGACCTCAATCCTGTTCATCACGACG
TGGCTGTCCTTCTCACTAGAAAGGACATCTGTGCTGGTTTCAATCGCCCCTGCGAGACCCTGGGCCTG
TCTCACCTTTCAGGAATGTGTCAGCCTCACCGCAGTTGTAACATCAATGAAGATTCGGGACTCCCTCT
GGCTTTCACAATTGCCCATGAGCTAGGACACAGCTTCGGCATCCAGCATGATGGGAAAGAAAATGACT
GTGAGCCTGTGGGCAGACATCCGTACATCATGTCCCGCCAGCTCCAGTACGATCCCACTCCGCTGACA
TGGTCCAAGTGCAGCGAGGAGTACATCACCCGCTTCTTGGACCGAGGCTGGGGGTTCTGTCTTGATGA
CATACCTAAAAAGAAAGGCTTGAAGTCCAAGGTCATTGCCCCCGGAGTGATCTATGATGTTCACCACC
AGTGCCAGCTACAATATGGACCCAATGCTACCTTCTGCCAGGAAGTAGAAAACGTCTGCCAGACACTG
TGGTGCTCCGTGAAGGGCTTTTGTCGCTCTAAGCTGGACGCTGCTGCAGATGGAACTCAATGTGGTGA
GAAGAAGTGGTGTATGGCAGGCAAGTGCATCACAGTGGGGAAGAAACCAGAGAGCATTCCTGGAGGCT
GGGGCCGCTGGTCACCCTGGTCCCACTGTTCCAGGACCTGTGGGGCTGGAGTCCAGAGCGCAGAGAGG
CTCTGCAACAACCCCGAGCCAAAGTTTGGAGGGAAATATTGCACTGGAGAAAGAAAACGCTATCGCTT
GTGCAACGTCCACCCCTGTCGCTCAGAGGCACCAACATTTCGGCAGATGCAGTGCAGTGAATTTGACA
CTGTTCCCTACAAGAATGAACTCTACCACTGGTTTCCCATTTTTAACCCAGCACATCCTTGTGAGCTC
TACTGCCGACCCATAGATGGCCAGTTTTCTGAGAAAATGCTGGATGCTGTCATTGATGGTACCCCTTG
CTTTGAAGGCGGCAACAGCAGAAATGTCTGTATTAATGGCATATGTAAGATGGTTGGCTGTGACTATG
AGATCGATTCCAATGCCACCGAGGATCGCTGCGGTGTGTGCCTGGGAGATGGCTCTTCCTGCCAGACT
GTGAGAAAGATGTTTAAGCAGAAGGAAGGATCTGGTTATGTTGACATTGGGCTCATTCCAAAAGGAGC
AAGGGACATAAGAGTGATGGAAATTGAGGGAGCTGGAAACTTCCTGGCCATCAGGAGTGAAGATCCTG
AAAAATATTACCTGAATGGAGGGTTTATTATCCAGTGGAACGGGAACTATAAGCTGGCAGGGACTGTC
TTTCAGTATGACAGGAAAGGAGACCTGGAAAAGCTGATGGCCACAGGTCCCACCAATGAGTCTGTGTG
GATCCAGCTTCTATTCCAGGTGACTAACCCTGGCATCAAGTATGAGTACACAATCCAGAAAGATGGCC
TTGACAATGATGTTGAGCAGCAGATGTACTTCTGGCAGTACGGCCACTGGACAGAGTGCAGTGTGACC
TGCGGGACAGGTATCCGCCGCCAAACTGCCCATTGCATAAAGAAGGGCCGCGGGATGGTGAAAGCTAC
ATTCTGTGACCCAGAAACACAGCCCAATGGGAGACAGAAGAAGTGCCATGAAAAGGCTTGTCCACCCA
GGTGGTGGGCAGGGGAGTGGGAAGCATGCTCGGCGACATGCGGGCCCCACGGGGAGAAGAAGCGAACC
GTGCTGTGCATCCAGACCATGGTCTCTGACGAGCAGGCTCTCCCGCCCACAGACTGCCAGCACCTGCT
GAAGCCCAAGACCCTCCTTTCCTGCAACAGAGACATCCTGTGCCCCTCGGACTGGACAGTGGGCAACT
GGAGTGAGTGTTCTGTTTCCTGTGGTGGTGGAGTGCGGATTCGCAGTGTCACATGTGCCAAGAACCAT
GATGAACCTTGCGATGTGACAAGGAAACCCAACAGCCGAGCTCTGTGTGGCCTCCAGCAATGCCCTTC
TAGCCGGAGAGTTCTGAAACCAAACAAAGGCACTATTTCCAATGGAAAAAACCCACCAACACTAAAGC
CCGTCCCTCCACCTACATCCAGGCCCAGAATGCTGACCACACCCACAGGGCCTGAGTCTATGAGCACA
AGCACTCCAGCAATCAGCAGCCCTAGTCCTACCACAGCCTCCAAAGAAGGAGACCTGGGTGGGAAACA
GTGGCAAGATAGCTCAACCCAACCTGAGCTGAGCTCTCGCTATCTCATTTCCACTGGAAGCACTTCCC
AGCCCATCCTCACTTCCCAATCCTTGAGCATTCAGCCAAGTGAGGAAAATGTTTCCAGTTCAGATACT
GGTCCTACCTCGGAGGGAGGCCTTGTAGCTACAACAACAAGTGGTTCTGGCTTGTCATCTTCCCGCAA
CCCTATCACTTGGCCTGTGACTCCATTTTACAATACCTTGACCAAAGGTCCAGAAATGGAGATTCACA
GTGGCTCAGGGGAAGAAAGAGAACAGCCTGAGGACAAAGATGAAAGCAATCCTGTAATATGGACCAAG
ATCAGAGTACCTGGAAATGACGCTCCAGTGGAAAGTACAGAAATGCCACTTGCACCTCCACTAACACC
AGATCTCAGCAGGGAGTCCTGGTGGCCACCCTTCAGCACAGTAATGGAAGGACTGCTCCCCAGCCAAA
GGCCCACTACTTCCGAAACTGGGACACCCAGAGTTGAGGGGATGGTTACTGAAAAGCCAGCCAACACT
CTGCTCCCTCTGGGAGGAGACCACCAGCCAGAACCCTCAGGAAAGACGGCAAACCGTAACCACCTGAA
ACTTCCAAACAACATGAACCAAACAAAAAGTTCTGAACCAGTCCTGACTGAGGAGGATGCAACAAGTC
TGATTACTGAGGGCTTTTTGCTAAATGCCTCCAATTACAAGCAGCTCACAAACGGCCACGGCTCTGCA
CACTGGATCGTCGGAAACTGGAGCGAGTGCTCCACCACATGTGGCCTGGGGGCCTACTGGAGAAGGGT
GGAGTGCAGCACCCAGATGGATTCTGACTGTGCGGCCATCCAGAGACCTGACCCTGCAAAAAGATGCC
ACCTCCGTCCCTGTGCTGGCTGGAAAGTGGGAAACTGGAGCAAGTGCTCCAGAAACTGCAGTGGGGGC
TTCAAGATACGCGAGATTCAGTGCGTGGACAGCCGGGACCACCGGAACCTGAGGCCATTTCACTGCCA
GTTCCTGGCCGGCATTCCTCCCCCATTGAGCATGAGCTGTAACCCGGAGCCCTGTGAGGCGTGGCAGG
TGGAGCCTTGGAGCCAGTGCTCCAGGTCCTGTGGAGGTGGAGTTCAGGAGAGAGGAGTGTTCTGTCCA
GGAGGCCTCTGTGATTGGACAAAAAGACCCACATCCACCATGTCTTGCAATGAGCACCTGTGCTGTCA
CTGGGCCACTGGGAACTGGGACCTGTGTTCCACTTCCTGTGGAGGTGGCTTTCAGAAGAGGACTGTCC
AATGTGTGCCCTCAGAGGGCAATAAAACTGAAGACCAAGACCAATGTCTATGTGATCACAAACCCAGA
CCTCCAGAATTCAAAAAATGCAACCAGCAGGCCTGCAAGAAAAGTGCCGATTTACTTTGCACTAAGGA
CAAACTGTCAGCCAGTTTCTGCCAGACACTGAAAGCCATGAAGAAATGTTCTGTGCCCACCGTGAGGG
CTGAGTGCTGCTTCTCGTGTCCCCAGACACACATCACACACACCCAAAGGCAAAGAAGGCAACGGTTG
CTCCAAAAGTCAAAAGGACTCCTCGAGGAGTTCTTTTGACTTTTGGAGCAACCGGGCCTGGCTGAGGC
TGTCTCCCATAAGATCT

NOV68f, 305867866

| | | | |
|---|---|---|---|
| Protein Sequence | SEQ ID NO: 1016 | 1576 aa | MW at 175822.7kD |

RSYGRQPQPGPVRFPDRRQEHFIKGLPEYHVVGPVRVDASGHFLSYGLHYPITSSRRKRDLDGSEDWV
YYRISHEEKDLFFNLTVNQGFLSNSYIMEKRYGNLSHVKMMASSAPLCHLSGTVLQQGTRVGTAALSA
CHGLTGFFQLPHGDFFIEPVKKHPLVEGGYHPHIVYRRQKVPETKEPTCGLKDSVNISQKQELWREKW
ERHNLPSRSLSRRSISKERWVETLVVADTKMIEYHGSENVESYILTIMNMVTGLFHNPSIGNAIHIVV
VRLILLEEEEQGLKIVHHAEKTLSSFCKWQKSINPKSDLNPVHHDVAVLLTRKDICAGFNRPCETLGL
SHLSGMCQPHRSCNINEDSGLPLAFTIAHELGHSFGIQHDGKENDCEPVGRHPYIMSRQLQYDPTPLT
WSKCSEEYITRFLDRGWGFCLDDIPKKKGLKSKVIAPGVIYDVHHQCQLQYGPNATFCQEVENVCQTL
WCSVKGFCRSKLDAAADGTQCGEKKWCMAGKCITVGKKPESIPGGWGRWSPWSHCSRTCGAGVQSAER
LCNNPEPKFGGKYCTGERKRYRLCNVHPCRSEAPTFRQMQCSEFDTVPYKNELYHWFPIFNPAHPCEL
YCRPIDGQFSEKMLDAVIDGTPCFEGGNSRNVCINGICKMVGCDYEIDSNATEDRCGVCLGDGSSCQT
VRKMFKQKEGSGYVDIGLIPKGARDIRVMEIEGAGNFLAIRSEDPEKYYLNGGFIIQWNGNYKLAGTV
FQYDRKGDLEKLMATGPTNESVWIQLLFQVTNPGIKYEYTIQKDGLDNDVEQQMYFWQYGHWTECSVT
CGTGIRRQTAHCIKKGRGNVKATFCDPETQPNGRQKKCHEKACPPRWWAGEWEACSATCGPHGEKKRT
VLCIQTMVSDEQALPPTDCQHLLKPKTLLSCNRDILCPSDWTVGNWSECSVSCGGGVRIRSVTCAKNH
DEPCDVTRKPNSRALCGLQQCPSSRRVLKPNKGTISNGKNPPTLKPVPPPTSRPRMLTTPTGPESMST
STPAISSPSPTTASKEGDLGGKQWQDSSTQPELSSRYLISTGSTSQPILTSQSLSIQPSEENVSSSDT
GPTSEGGLVATTTSGSGLSSSRNPITWPVTPFYNTLTKGPEMEIHSGSGEEREQPEDKDESNPVIWTK
IRVPGNDAPVESTEMPLAPPLTPDLSRESWWPPFSTVMEGLLPSQRPTTSETGTPRVEGMVTEKPAWI
LLPLGGDHQPEPSGKTANRNHLKLPNNMNQTKSSEPVLTEEDATSLITEGFLLNASNYKQLTNGHGSA
HWIVGNWSECSTTCGLGAYWRRVECSTQMDSDCAAIQRPDPAKRCHLRPCAGWKVGNWSKCSRNCSGG
FKIREIQCVDSRDHRNLRPFHCQFLAGIPPPLSMSCNPEPCEAWQVEPWSQCSRSCGGGVQERGVFCP
GGLCDWTKRPTSTMSCNEHLCCHWATGNWDLCSTSCGGGFQKRTVQCVPSEGNKTEDQDQCLCDHKPR
PPEFKKCNQQACKKSADLLCTKDKLSASFCQTLKAMKKCSVPTVRAECCFSCPQTHITHTQRQRRQRL
LQKSKGLLEEFF

| | | |
|---|---|---|
| NOV68g, 318176397 | SEQ ID NO: 1017 | 3174 bp |

| | | |
|---|---|---|
| DNA Sequence | ORF Start: at 1 | ORF Stop: end of sequence |

CTGGAATGCGCCCTTAGATCTGGCCGCTGGTCACCCTGGTCCCACTGTTCCAGGACCTGTGGGGCTGG
AGTCCAGAGCGCAGAGAGGCTCTGCAACAACCCCGAGCCAAAGTTTGGAGGGAAATATTGCACTGGAG
AAAGAAAACGCTATCGCTTGTGCAACGTCCACCCCTGTCGCTCAGAGGCACCAACATTTCGGCAGATG
CAGTGCAGTGAATTTGACACTGTTCCCTACAAGAATGAACTCTACCACTGGTTTCCCATTTTTAACCC
AGCACATCCTTGTGAGCTCTACTGCCGACCCATAGATGGCCAGTTTTCTGAGAAAATGCTGGATGCTG
TCATTGATGGTACCCCTTGCTTTGAAGGCGGCAACAGCAGAAATGTCTGTATTAATGGCATATGTAAG
ATGGTTGGCTGTGACTATGAGATCGATTCCAATGCCACCGAGGATCGCTGCGGTGTGTGCCTGGGAGA
TGGCTCTTCCTGCCAGACTGCGAGAAAGATGTTTAAGCAGAAGGAAGGATCTGGTTATGTTGACATTG
GGCTCATTCCAAAAGGAGCAAGGGACATAAGAGTGATGGAAATTGAGGGAGCTGGAAACTTCCTGGCC
ATCAGGAGTGAAGATCCTGAAAAATATTACCTGAATGGAGGGTTTATTATCCAGTGGAACGGGAACTA
TAAGCTGGCAGGGACTGTCTTTCAGTATGACAGGAAAGGAGACCTGGAAAAGCTGATGGCCACAGGTC
CCACCAATGAGTCTGTGTGGATCCAGCTTCTATTCCAGGTGACTAACCCTGGCATCAAGTATGAGTAC
ACAATCCAGAAAGATGGCCTTGACAATGATGTTGAGCAGCAGATGTACTTCTGGCAGTACGGCCACTG
GACAGAGTGCAGTGTGACCTGCGGGACAGGTATCCGCCGCCAAACTGCCCATTGCATAAAGAAGGGCC
GCGGGATGGTGAAAGCTACATTCTGTGACCCAGAAACACAGCCCAATGGGAGACAGAAGAAGTGCCAT
GAAAAGGCTTGTCCACCCAGGTGGTGGGCAGGGGAGTGGGAAGCATGCTCGGCGACATGCGGGCCCCA
CGGGGAGAAGAAGCGAACCGTGcTGTGCATCCAGACCATGGTCTCTGACGAGCAGGCTCTCCCGCCCA
CAGACTGCCAGCACCTGCTGAAGCCCAAGACCCTCCTTTCCTGCAACAGAGACATCCTGTGCCCCTCG
GACTGGACAGTGGGCAACTGGAGTGAGTGTTCTGTTTCCTGTGGTGGTGGAGTGCGGATTCGCAGTGT
CACATGTGCCAAGAACCATGATGAACCTTGCGATGTGACAAGGAAACCCAACAGCCGAGCTCTGTGTG
GCCTCCAGCAATGCCCTTCTAGCCGGAGAGTTCTGAAACCAAACAAAGGCACTATTTCCAATGGAAAA
AACCCACCAACACTAAAGCCCGTCCCTCCACCTACATCCAGGCCCAGAATGCTGACCACACCCACAGG
GCCTGAGTCTATGAGCACAAGCACTCCGGCAATCAGCAGCCCTAGTCCTACCACAGCCTCCAAAGAAG
GAGACCTGGGTGGGAAACAGTGGCAAGATAGCTCAACCCAACCTGAGCTGAGCTCTCGCTATCTCATT
TCCACTGGAAGCACTTCCCAGCCCATCCTCACTTCCCAATCCTTGAGCATTCAGCCAAGTGAGGAAAA
TGTTTCCAGTTCAGATACTGGTCCTACCTCGGAGGGAGGCCTTGTAGCTACAACAACAAGTGGTTCTG
GCTTGTCATCTTCCCGCAACCCTATCACTTGGCCTGTGACTCCATTTTACAATACCTTGACCAAAGGT
CCAGAAATGGAGATTCACAGTGGCTCAGGGGAAGAAAGAGAACAGCCTGAGGACAAAGATGAAAGCAA
TCCTGTAATATGGACCAAGATCAGAGTACCTGGAAATGACGCTCCAGTGGAAAGTACAGAAATGCCAC
TTGCACCTCCACTAACACCAGATCTCAGCAGGGAGTCCTGGTGGCCACCCTTCAGCACAGTAATGGAA
GGACTGCTCCCCAGCCAAAGGCCCACTACTTCCGAAACTGGGACACCCAGAGTTGAGGGGATGGTTAC
TGAAAAGCCAGCCAACACTCTGCTCCCTCTGGGAGGAGACCACCAGCCAGAACCCTCAGGAAAGACGG
CAAACCGTAACCACCTGAAACTTCCAAACAACATGAACCAAACAAAAAGTTCTGAACCAGTCCTGACT
GAGGAGGATGCAACAAGTCTGATTACTGAGGGCTTTTTGCTAAATGCCTCCAATTACAAGCAGCTCAC
AAACGGCTACGGCTCTGCACACTGGATCGTCGGAAACTGGAGCGAGTGCTCCACCACATGTGGCCTGG
GGGCCTACTGGAGAAGGGTGGAGTGCAGCACCCAGATGGATTCTGACTGTGCGGCCATCCAGAGACCT
GACCCTGCAAAAAGATGCCACCTCCGTCCCTGTGCTGGCTGGAAAGTGGGAAACTGGAGCAAGTGCTC
CAGAAACTGCAGTGGGGGCTTCAAGATACGCGAGATTCAGTGCGTGGACAGCCGGGACCACCGGAACC
TGAGGCCATTTCACTGCCAGTTCCTGGCCGGCATTCCTCCCCCATTGAGCATGAGCTGTAACCCGGAG
CCCTGTGAGGCGTGGCAGGTGGAGCCTTGGAGCCAGTGCTCCAGGTCCTGTGGAGGTGGAGTTCAGGA
GAGAGGAGTGTTCTGTCCAGGAGGCCTCTGTGATTGGACAAAAAGACCCACATCCACCATGTCTTGCA
ATGAGCACCTGTGCTGTCACTGGGCCACTGGGAACTGGGACCTGTGTTCCACTTCCTGTGGAGGCGGC
TTTCAGAAGAGGACTGTCCAATGTGTGCCCTCAGAGGGCAATAAAACTGAAGACCAAGACCAATGTCT
ATGTGATCACAAACCCAGACCTCCAGAATTCAAAAAATGCAACCAGCAGGCCTGCAAGAAAAGTGCCG
ATTTACTTTGCACTAAGGACAAACTGTCAGCCAGTTTCTGCCAGACACTGAAAGCCATGAAGAAATGT
TCTGTGCCCACCGTGAGGGCTGAGTGCTGCTTCTCGTGTCCCCAGACACACATCACACACACCCAAAG
GCAAAGAAGGCAACGGTTGCTCCAAAAGTCAAAAGAACTCCTCGAG

NOV68g, 318176397

| | | | |
|---|---|---|---|
| Protein Sequence | SEQ ID NO: 1018 | 1058 aa | MW at 117062.0kD |

LECALRSGRWSPWSHCSRTCGAGVQSAERLCNNPEPKFGGKYCTGERKRYRLCNVHPCRSEAPTFRQM
QCSEFDTVPYKNELYHWFPIFNPAHPCELYCRPIDGQFSEKMLDAVIDGTPCFEGGNSRNVCINGICK
MVGCDYEIDSNATEDRCGVCLGDGSSCQTARKMFKQKEGSGYVDIGLIPKGARDIRVMEIEGAGNFLA
IRSEDPEKYYLNGGFIIQWNGNYKLAGTVFQYDRKGDLEKLMATGPTNESVWIQLLFQVTNPGIKYEY
TIQKDGLDNDVEQQMYFWQYGHWTECSVTCGTGIRRQTAHCIKKGRGMVKATFCDPETQPNGRQKKCH
EKACPPRWWAGEWEACSATCGPHGEKKRTVLCIQTMVSDEQALPPTDCQHLLKPKTLLSCNRDILCPS
DWTVGNWSECSVSCGGGVRIRSVTCAKNHDEPCDVTRKPNSRALCGLQQCPSSRRVLKPNKGTISNGK
NPPTLKPVPPPTSRPRMLTTPTGPESMSTSTPAISSPSPTTASKEGDLGGKQWODSSTQPELSSRYLI
STGSTSQPILTSQSLSIQPSEENVSSSDTGPTSEGGLVATTTSGSGLSSSRNPITWPVTPFYNTLTKG
PEMEIHSGSGEEREOPEDKDESNPVIWTKIRVPGNDAPVESTEMPLAPPLTPDLSRESWWPPFSTVME
GLLPSQRPTTSETGTPRVEGMVTEKPANTLLPLGGDHQPEPSGKTANRNHLKLPNNMNQTKSSEPVLT
EEDATSLITEGFLLNASNYKQLTNGYGSAHWIVGNWSECSTTCGLGAYWRRVECSTQMDSDCAAIQRP
DPAKRCHLRPCAGWKVGNWSKCSRNCSGGFKIREIQCVDSRDHRNLRPFHCQFLAGIPPPLSMSCNPE
PCEAWQVEPWSQCSRSCGGGVQERGVFCPGGLCDWTKRPTSTMSCNEHLCCHWATGNWDLCSTSCGGG
FQKRTVQCVPSEGNKTEDQDQCLCDHKPRPPEFKKCNQQACKKSADLLCTKDKLSASFCQTLKANKKC
SVPTVRAECCFSCPQTHITHTQRQRRQRLLQKSKELLE

| | | |
|---|---|---|
| NOV68h, CG58504-02 | SEQ ID NO: 1019 | 1068 bp |

| | | |
|---|---|---|
| DNA Sequence | ORF Start: at 7 | ORF Stop: at 1063 |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCTCAGTGGCACGGTTCTACAGC
AGGGCACCAGAGTTGGGATGGCAGCCCTCAGTGCCTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAACTTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTGCATCCTCACCATCATGAACATGGTCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68h, CG58504-02

| | | | |
|---|---|---|---|
| Protein Sequence | SEQ ID NO: 1020 | 352 aa | MW at 39853.3kD |

NLTVNQGFLSNSYIMEKRYGNLSHVKMMASSAPLCHLSGTVLQQGTRVGMAALSACHGLTGFFQLPHG
DFFIEPVKKHPLVEGGYHPHIVYRRQKVPETKEPTCGLKDSVNISQKQELWREKWERHNLPSRSLSRR
SISKERWVETLVVADTKMIEYHGSENVESCILTIMNMVTGLFHNPSIGNAIHIVVVRLILLEEEEQGL
KIVHHAEKTLSSFCKWQKSINPKSDLNPVHHDVAVLLTRKDICAGFNRPCETLGLSHLSGMCQPHRSC
NINEDSGLPLAFTIAHELGHSFGIQHDGKENDCEPVGRHPYIMSRQLQYDPTPLTWSKCSEEYITRFL
DRGWGFCLDDIP

| | | |
|---|---|---|
| NOV68i, CG58504-03 | SEQ ID NO: 1021 | 1068 bp |

| | | |
|---|---|---|
| DNA Sequence | ORF Start: at 7 | ORF Stop: at 1063 |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCTCAGTGGCACGGTTCTACAGC
AGGGCACCAGAGTTGGGACGGCAGCCCTCAGTGCcTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAACTTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTACATCCTCACCATCATGAACATGATCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68i, CG58504-03

| | | | |
|---|---|---|---|
| Protein Sequence | SEQ ID NO: 1022 | 352 aa | MW at 39897.2kD |

NLTVNQGFLSNSYIMEKRYGNLSHVKMMASSAPLCHLSGTVLQQGTRVGTAALSACHGLTGFFQLPHG
DFFIEPVKKHPLVEGGYHPHIVYRRQKVPETKEPTCGLKDSVNISQKQELWREKWERHNLPSRSLSRR
SISKERWVETLVVADTKMIEYHGSENVESYILTIMNNITGLFHNPSIGNAIHIVVVRLILLEEEEQGL
KIVHHAEKTLSSFCKWQKSINPKSDLNPVHHDVAVLLTRKDICAGFNRPCETLGLSHLSGMCQPHRSC
NINEDSGLPLAFTIAHELGHSFGIQHDGKENDCEPVGRHPYIMSRQLQYDPTPLTWSKCSEEYITRFL
DRGWGFCLDDIP

| | | |
|---|---|---|
| NOV68j, CG58504-04 | SEQ ID NO: 1023 | 252 bp |

| | | |
|---|---|---|
| DNA Sequence | ORF Start: at 7 | ORF Stop: at 247 |

AAGCTTCACCAGTGCCAGCTACAATATGGACCCAATGCTACCTTCTGCCAGGAAGTAGAAAACGTCTG
CCAGACACTGTGGTGCTCCGTGAAGGGCTTTTGTCGCTCTAAGCTGGACGCTGCTGCAGATGGAACTC
AATGTGGTGAGAAGAAGTGGTGTATGGCAGGCAAGTGCATCACAGTGGGGAAGAAACCAGAGAGCATT
CCTGGAGGCTGCGGCCGCTGGTCACCCTGGTCCCACTGTTCCCTCGAG

NOV68j, CG58504-04

| | | | |
|---|---|---|---|
| Protein Sequence | SEQ ID NO: 1024 | 80 aa | MW at 8757.0kD |

HQCQLQYGPNATFCQEVENVCQTLWCSVKGFCRSKLDAAADGTQCGEKKWCMAGKCITVGKKPESIPG
GCGRWSPWSHCS

| | | |
|---|---|---|
| NOV68k, CG58504-05 | SEQ ID NO: 1025 | 4777 bp |

| | | |
|---|---|---|
| DNA Sequence | ORF Start: at 7 | ORF Stop: TGA at 4729 |

AGATCTTATGGGAGACAGCCTCAGCCAGGCCCGGTTCGCTTCCCGGACAGGAGGCAAGAGCATTTTAT
CAAGGGCCTGCCAGAATACCACGTGGTGGGTCCAGTCCGAGTAGATGCCAGTGGGCATTTTTTGTCAT
ATGGCTTGCACTATCCCATCACGAGCAGCAGGAGGAAGAGAGATTTGGATGGCTCAGAGGACTGGGTG
TACTACAGAATTTCTCACGAGGAGAAGGACCTGTTTTTTAACTTGACGGTCAATCAAGGATTTCTTTC
CAATAGCTACATCATGGAGAAGAGATATGGGAACCTCTCCCATGTTAAGATGATGGCTTCCTCTGCCC
CCCTCTGCCATCTCAGTGGCACGGTTCTACAGCAGGGCACCAGAGTTGGGACGGCAGCCCTCAGTGCC
TGCCATGGACTGACTGGATTTTTCCAACTACCACATGGAGACTTTTTCATTGAACCCGTGAAGAAGCA
TCCAcTGGTTGAGGGAGGGTACCACCCGCACATCGTTTACAGGAGGCAGAAAGTTCCAGAAACCAAGG
AGCCAACCTGTGGATTAAAGGACAGTGTTAACATCTCCCAGAAGCAAGAGCTATGGCGGGAGAAGTGG
GAGAGGCACAACTTGCCAAGCAGAAGCCTCTCTCGGCGTTCCATCAGCAAGGAGAGATGGGTGGAGAC
ACTGGTGGTGGCCGACACAAAGATGATTGAATACCATGGGAGTGAGAATGTGGAGTCCTACATCCTCA
CCATCATGAACATGGTCACTGGGTTGTTCCATAACCCAAGCATTGGCAATGCAATTCACATTGTTGTG
GTTCGGCTCATTCTACTCGAAGAAGAAGAGCAAGGACTGAAAATAGTTCACCATGCAGAAAAGACACT
GTCTAGCTTCTGCAAGTGGCAGAAGAGTATCAATCCCAAGAGTGACCTCAATCCTGTTCATCACGACG
TGGCTGTCCTTCTCACTAGAAAGGACATCTGTGCTGGTTTCAATCGCCCCTGCGAGACCCTGGGCCTG
TCTCACCTTTCAGGAATGTGTCAGCCTCACCGCAGTTGTAACATCAATGAAGATTCGGGACTCCCTCT
GGCTTTCACAATTGCCCATGAGCTAGGACACAGCTTCGGCATCCAGCATGATGGGAAAGAAAATGACT
GTGAGCCTGTGGGCAGACATCCGTACATCATGTCCCGCCAGCTCCAGTACGATCCCACTCCGCTGACA
TGGTCCAAGTGCAGCGAGGAGTACATCACCCGCTTCTTGGACCGAGGCTGGGGGTTCTGTCTTGATGA
CATACCTAAAAAGAAAGGCTTGAAGTCCAAGGTCATTGCCCCCGGAGTGATCTATGATGTTCACCACC
AGTGCCAGCTACAATATGGACCCAATGCTACCTTCTGCCAGGAAGTAGAAAACGTCTGCCAGACACTG
TGGTGCTCCGTGAAGGGCTTTTGTCGCTCTAAGCTGGACGCTGCTGCAGATGGAACTCAATGTGGTGA
GAAGAAGTGGTGTATGGCAGGCAAGTGCATCACAGTGGGGAAGAAACCAGAGAGCATTCCTGGAGGCT
GGGGCCGCTGGTCACCCTGGTCCCACTGTTCCAGGACCTGTGGGGCTGGAGTCCAGAGCGCAGAGAGG
CTCTGCAACAACCCCGAGCCAAAGTTTGGAGGGAAATATTGCACTGGAGAAAGAAAACGCTATCGCTT
GTGCAACGTCCACCCCTGTCGCTCAGAGGCACCAACATTTCGGCAGATGCAGTGCAGTGAATTTGACA
CTGTTCCCTACAAGAATGAACTCTACCACTGGTTTCCCATTTTTAACCCAGCACATCCTTGTGAGCTC
TACTGCCGACCCATAGATGGCCAGTTTTCTGAGAAAATGCTGGATGCTGTCATTGATGGTACCCCTTG
CTTTGAAGGCGGCAACAGCAGAAATGTCTGTATTAATGGCATATGTAAGATGGTTGGCTGTGACTATG
AGATCGATTCCAATGCCACCGAGGATCGCTGCGGTGTGTGCCTGGGAGATGGCTCTTCCTGCCAGACT
GTGAGAAAGATGTTTAAGCAGAAGGAAGGATCTGGTTATGTTGACATTGGGCTCATTCCAAAAGGAGC
AAGGGACATAAGAGTGATGGAAATTGAGGGAGCTGGAAACTTCCTGGCCATCAGGAGTGAAGATCCTG
AAAAATATTACCTGAATGGAGGGTTTATTATCCAGTGGAACGGGAACTATAAGCTGGCAGGGACTGTC
TTTCAGTATGACAGGAAAGGAGACCTGGAAAAGCTGATGGCCACAGGTCCCACCAATGAGTCTGTGTG
GATCCAGCTTCTATTCCAGGTGACTAACCCTGGCATCAAGTATGAGTACACAATCCAGAAAGATGGCC
TTGACAATGATGTTGAGCAGCAGATGTACTTCTGGCAGTACGGCCACTGGACAGAGTGCAGTGTGACC
TGCGGGACAGGTATCCGCCGCCAAACTGCCCATTGCATAAAGAAGGGCCGCGGGATGGTGAAAGCTAC
ATTCTGTGACCCAGAAACACAGCCCAATGGGAGACAGAAGAAGTGCCATGAAAAGGCTTGTCCACCCA
GGTGGTGGGCAGGGGAGTGGGAAGCATGCTCGGCGACATGCGGGCCCCACGGGGAGAAGAAGCGAACC
GTGCTGTGCATCCAGACCATGGTCTCTGACGAGCAGGCTCTCCCGCCCACAGACTGCCAGCACCTGCT
GAAGCCCAAGACCCTCCTTTCCTGCAACAGAGACATCCTGTGCCCCTCGGACTGGACAGTGGGCAACT
GGAGTGAGTGTTCTGTTTCCTGTGGTGGTGGAGTGCGGATTCGCAGTGTCACATGTGCCAAGAACCAT
GATGAACCTTGCGATGTGACAAGGAAACCCAACAGCCGAGCTCTGTGTGGCCTCCAGCAATGCCCTTC
TAGCCGGAGAGTTCTGAAACCAAACAAAGGCACTATTTCCAATGGAAAAAACCCACCAACACTAAAGC
CCGTCCCTCCACCTACATCCAGGCCCAGAATGCTGACCACACCCACAGGGCCTGAGTCTATGAGCACA
AGCACTCCAGCAATCAGCAGCCCTAGTCCTACCACAGCCTCCAAAGAAGGAGACCTGGGTGGGAAACA
GTGGCAAGATAGCTCAACCCAACCTGAGCTGAGCTCTCGCTATCTCATTTCCACTGGAAGCACTTCCC
AGCCCATCCTCACTTCCCAATCCTTGAGCATTCAGCCAAGTGAGGAAAATGTTTCCAGTTCAGATACT
GGTCCTACCTCGGAGGGAGGCCTTGTAGCTACAACAACAAGTGGTTCTGGCTTGTCATCTTCCCGCAA
CCCTATCACTTGGCCTGTGACTCCATTTTACAATACCTTGACCAAAGGTCCAGAAATGGAGATTCACA
GTGGCTCAGGGGAAGAAAGAGAACAGCCTGAGGACAAAGATGAAAGCAATCCTGTAATATGGACCAAG
ATCAGAGTACCTGGAAATGACGCTCCAGTGGAAAGTACAGAAATGCCACTTGCACCTCCACTAACACC
AGATCTCAGCAGGGAGTCCTGGTGGCCACCCTTCAGCACAGTAATGGAAGGACTGCTCCCCAGCCAAA
GGCCCACTACTTCCGAAACTGGGACACCCAGAGTTGAGGGGATGGTTACTGAAAAGCCAGCCAACACT
CTGCTCCCTCTGGGAGGAGACCACCAGCCAGAACCCTCAGGAAAGACGGCAAACCGTAACCACCTGAA
ACTTCCAAACAACATGAACCAAACAAAAAGTTCTGAACCAGTCCTGACTGAGGAGGATGCAACAAGTC
TGATTACTGAGGGCTTTTTGCTAAATGCCTCCAATTACAAGCAGCTCACAAACGGCCACGGCTCTGCA
CACTGGATCGTCGGAAACTGGAGCGAGTGCTCCACCACATGTGGCCTGGGGGCCTACTGGAGAAGGGT
GGAGTGCAGCACCCAGATGGATTCTGACTGTGCGGCCATCCAGAGACCTGACCCTGCAAAAAGATGCC
ACCTCCGTCCCTGTGCTGGCTGGAAAGTGGGAAACTGGAGCAAGTGCTCCAGAAACTGCAGTGGGGGC
TTCAAGATACGCGAGATTCAGTGCGTGGACAGCCGGGACCACCGGAACCTGAGGCCATTTCACTGCCA
GTTCCTGGCCGGCATTCCTCCCCCATTGAGCATGAGCTGTAACCCGGAGCCCTGTGAGGCGTGGCAGG
TGGAGCCTTGGAGCCAGTGCTCCAGGTCCTGTGGAGGTGGAGTTCAGGAGAGAGGAGTGTTCTGTCCA
GGAGGCCTCTGTGATTGGACAAAAAGACCCACATCCACCATGTCTTGCAATGAGCACCTGTGCTGTCA
CTGGGCCACTGGGAACTGGGACCTGTGTTCCACTTCCTGTGGAGGTGGCTTTCAGAAGAGGACTGTCC
AATGTGTGCCCTCAGAGGGCAATAAAACTGAAGACCAAGACCAATGTCTATGTGATCACAAACCCAGA
CCTCCAGAATTCAAAAAATGCAACCAGCAGGCCTGCAAGAAAAGTGCCGATTTACTTTGCACTAAGGA
CAAACTGTCAGCCAGTTTCTGCCAGACACTGAAAGCCATGAAGAAATGTTCTGTGCCCACCGTGAGGG
CTGAGTGCTGCTTCTCGTGTCCCCAGACACACATCACACACACCCAAAGGCAAAGAAGGCAACGGTTG
CTCCAAAAGTCAAAAGGACTCCTCGAGGAGTTCTTTTGACTTTTGGAGCAACCGGGCCTGGCTGAGGC
TGTCTCCCATAAGATCT

NOV68k, CG58504-05

| | | | |
|---|---|---|---|
| Protein Sequence | SEQ ID NO: 1026 | 1574 aa | MW at 175579.4kD |

YGRQPQPGPVRFPDRRQEHFIKGLPEYHVVGPVRVDASGHFLSYGLHYPITSSRRKRDLDGSEDWVYY
RISHEEKDLFFNLTVNQGFLSNSYIMEKRYGNLSHVKMMASSAPLCHLSGTVLQQGTRVGTAALSACH
GLTGFFQLPHGDFFIEPVKKHPLVEGGYHPHIVYRRQKVPETKEPTCGLKDSVNISQKQELWREKWER
HNLPSRSLSRRSISKERWVETLVVADTKMIEYHGSENVESYILTIMNMVTGLFHNPSIGNAIHIVVVR
LILLEEEEQGLKIVHHAEKTLSSFCKWQKSINPKSDLNPVHHDVAVLLTRKDICAGFNRPCETLGLSH
LSGMCQPHRSCNINEDSGLPLAFTIAHELGHSFGIQHDGKENDCEPVGRHPYIMSRQLQYDPTPLTWS
KCSEEYITRFLDRGWGFCLDDIPKKKGLKSKVIAPGVIYDVHHQCQLQYGPNATFCQEVENVCQTLWC
SVKGFCRSKLDAAADGTQCGEKKWCMAGKCITVGKKPESIPGGWGRWSPWSHCSRTCGAGVOSAERLC
NNPEPKFGGKYCTGERKRYRLCNVHPCRSEAPTFRQMQCSEFDTVPYKNELYHWFPIFNPAHPCELYC
RPIDGQFSEKMLDAVIDGTPCFEGGNSRNVCINGICKMVGCDYEIDSNATEDRCGVCLGDGSSCQTVR
KMFKQKEGSGYVDIGLIPKGARDIRVMEIEGAGNFLAIRSEDPEKYYLNGGFIIQWNGNYKLAGTVFQ
YDRKGDLEKLMATGPTNESVWIQLLFQVTNPGIKYEYTIQKDGLDNDVEQQMYFWQYGHWTECSVTCG
TGIRRQTAHCIKKGRGMVKATFCDPETQPNGRQKKCHEKACPPRWWAGEWEACSATCGPHGEKKRTVL
CIQTMVSDEQALPPTDCQHLLKPKTLLSCNRDILCPSDWTVGNWSECSVSCGGGVRIRSVTCAKNHDE
PCDVTRKPNSRALCGLQQCPSSRRVLKPNKGTISNGKNPPTLKPVPPPTSRPRMLTTPTGPESMSTST
PAISSPSPTTASKEGDLGGKQWQDSSTQPELSSRYLISTGSTSQPILTSQSLSIQPSEENVSSSDTGP
TSEGGLVATTTSGSGLSSSRNPITWPVTPFYNTLTKGPEMEIHSGSGEEREQPEDKDESNPVIWTKIR
VPGNDAPVESTEMPLAPPLTPDLSRESWWPPFSTVMEGLLPSQRPTTSETGTPRVEGMVTEKPANTLL
PLGGDHQPEPSGKTANRNHLKLPNNNNQTKSSEPVLTEEDATSLITEGFLLNASNYKQLTNGHGSAHW
IVGNWSECSTTCGLGAYWRRVECSTQMDSDCAAIQRPDPAKRCHLRPCAGWKVGNWSKCSRNCSGGFK
IREIQCVDSRDHRNLRPFHCQFLAGIPPPLSMSCNPEPCEAWQVEPWSQCSRSCGGGVQERGVFCPGG
LCDWTKRPTSTMSCNEHLCCHWATGNWDLCSTSCGGGFQKRTVQCVPSEGNKTEDQDQCLCDHKPRPP
EFKKCNQQACKKSADLLCTKDKLSASFCQTLKAMKKCSVPTVRAECCFSCPQTHITHTQRQRRQRLLQ
KSKGLLEEFF

| | | |
|---|---|---|
| NOV68l, CG58504-06 | SEQ ID NO: 1027 | 1068 bp |

| | | |
|---|---|---|
| DNA Sequence | ORF Start: at 1 | ORF Stop: end of sequence |

AAGCTTAACTTGACGGTCAATCAAGGATTTCTTTCCAATAGCTACATCATGGAGAAGAGATATGGGAA
CCTCTCCCATGTTAAGATGATGGCTTCCTCTGCCCCCCTCTGCCATCTCAGTGGCACGGTTCTACAGC
AGGGCACCAGAGTTGGGACGGCAGCCCTCAGTGCCTGCCATGGACTGACTGGATTTTTCCAACTACCA
CATGGAGACTTTTTCATTGAACCCGTGAAGAAGCATCCACTGGTTGAGGGAGGGTACCACCCGCACAT
CGTTTACAGGAGGCAGAAAGTTCCAGAAACCAAGGAGCCAACCTGTGGATTAAAGGACAGTGTTAACA
TCTCCCAGAAGCAAGAGCTATGGCGGGAGAAGTGGGAGAGGCACAACTTGCCAAGCAGAAGCCTCTCT
CGGCGTTCCATCAGCAAGGAGAGATGGGTGGAGACACTGGTGGTGGCCGACACAAAGATGATTGAATA
CCATGGGAGTGAGAATGTGGAGTCCTACATCCTCACCATCATGAACATGGTCACTGGGTTGTTCCATA
ACCCAAGCATTGGCAATGCAATTCACATTGTTGTGGTTCGGCTCATTCTACTCGAAGAAGAAGAGCAA
GGACTGAAAATAGTTCACCATGCAGAAAAGACACTGTCTAGCTTCTGCAAGTGGCAGAAGAGTATCAA
TCCCAAGAGTGACCTCAATCCTGTTCATCACGACGTGGCTGTCCTTCTCACCAGAAAGGACATCTGTG
CTGGTTTCAATCGCCCCTGCGAGACCCTGGGCCTGTCTCACCTTTCAGGAATGTGTCAGCCTCACCGC
AGTTGTAACATCAATGAAGATTCGGGACTCCCTCTGGCTTTCACAATTGCCCATGAGCTAGGACACAG
CTTCGGCATCCAGCATGATGGGAAAGAAAATGACTGTGAGCCTGTGGGCAGACATCCGTACATCATGT
CCCGCCAGCTCCAGTACGATCCCACTCCGCTGACATGGTCCAAGTGCAGCGAGGAGTACATCACCCGC
TTCTTGGACCGAGGCTGGGGGTTCTGTCTTGATGACATACCTCTCGAG

NOV68l, CG58504-06

| | | | |
|---|---|---|---|
| Protein Sequence | SEQ ID NO: 1028 | 356 aa | MW at 40366.8kD |

KLNLTVNQGFLSNSYIMEKRYGNLSHVKNMASSAPLCHLSGTVLQOGTRVGTAALSACHGLTGFFQLP
HGDFFIEPVKKHPLVEGGYHPHIVYRRQKVPETKEPTCGLKDSVNISQKQELWREKWERHNLPSRSLS
RRSISKERWVETLVVADTKMIEYHGSENVESYILTIMNNVTGLFHNPSIGNAIHIVVVRLILLEEEEQ
GLKIVHHAEKTLSSFCKWQKSINPKSDLNPVHHDVAVLLTRKDICAGFNRPCETLGLSHLSGMCQPHR
SCNINEDSGLPLAFTIAHELGHSFGIQHDGKENDCEPVGRHPYIMSRQLQYDPTPLTWSKCSEEYITR
FLDRGWGFCLDDIPLE

| | | |
|---|---|---|
| NOV68m, CG58504-07 | SEQ ID NO: 1029 | 252 bp |

| | | |
|---|---|---|
| DNA Sequence | ORF Start: at 1 | ORF Stop: end of sequence |

AAGCTTCACCAGTGCCAGCTACAATATGGACCCAATGCTACCTTCTGCCAGGAAGTAGAAAACGTCTG
CCAGACACTGTGGTGCTCCGTGAAGGGCTTTTGTCGCTCTAAGCTGGACGCTGCTGCAGATGGAACTC
AATGTGGTGAGAAGAAGTGGTGTATGGCAGGCAAGTGCATCACAGTGGGGAAGAAACCAGAGAGCATT
CCTGGAGGCTGGGGCCGCTGGTCACCCTGGTCCCACTGTTCCCTCGAG

NOV68m, CG58504-07

| | | | |
|---|---|---|---|
| Protein Sequence | SEQ ID NO: 1030 | 84 aa | MW at 9323.7kD |

KLHQCQLQYGPNATFCQEVENVCQTLWCSVKGFCRSKLDAAADGTQCGEKKWCMAGKCITVGKKPESI
PGGWGRWSPWSHCSLE

A ClustalW comparison of the above protein sequences yields the following sequence alignment shown in Table 68B.

TABLE 68B
Comparison of the NOV68 protein sequences.

| | |
|---|---|
| NOV68a | MPCAQRSWLANLSVVAQLLNFGALCYGRQPQPGPVRFPDRRQEHFIKGLPEYHVVGPVRV |
| NOV68b | ------------------------------------------------------------ |
| NOV68c | ------------------------------------------------------------ |
| NOV68d | ------------------------------------------------------------ |
| NOV68e | ------------------------------------------------------------ |
| NOV68f | ------------------------------------------------------------ |
| NOV68g | ------------------------------------------------------------ |
| NOVS8h | ------------------------------------------------------------ |
| NOV68i | ------------------------------------------------------------ |
| NOV68j | ------------------------------------------------------------ |
| NOV68k | ------------------------------------------------------------ |
| NOV68l | ------------------------------------------------------------ |
| NOV68m | ------------------------------------------------------------ |
| NOV68a | DASGHFLSYGLHYPITSSRRKRDLDGSEDWVYYRISHEEKDLFFNLTVNQGFLSNSYIME |
| NOV68b | ------------------------------------------------------------ |
| NOV68c | ------------------------------------------------------------ |
| NOV68d | ------------------------------------------------------------ |
| NOV68e | ------------------------------------------------------------ |
| NOV68f | ------------------------------------------------------------ |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | ------------------------------------------------------------ |
| NOV68l | ------------------------------------------------------------ |
| NOV68m | ------------------------------------------------------------ |
| NOV68a | KRYGNLSHVKMMASSAPLCHLSGTVLQQGTRVGTAALSACHGLTGFFQLPHGDFFIEPVK |
| NOV68b | ------------------------------------------------------------ |
| NOV68c | ------------------------------------------------------------ |
| NOV68d | ------------------------------------------------------------ |
| NOV68e | ------------------------------------------------------------ |
| NOV68f | ------------------------------------------------------------ |
| NOV68g | --------------------------------LECALRSGRWSPWSHCSRTCGAGVQSAE |
| NOV68h | ------------------------------------------------------------ |
| NOV68i | ------------------------------------------------------------ |
| NOV68j | ------------------------------------------------------------ |
| NOV68k | ------------------------------------------------------------ |
| NOV68l | ------------------------------------------------------------ |
| NOV68m | ------------------------------------------------------------ |
| NOV68a | KHPLVEGGYHPHIVYRRQKVPETKEPTCGLKDSVNISQKQELWREKWERHNLPSRSLSRR |
| NOV68b | ------------------------------------------------------------ |
| NOV68c | ------------------------------------------------------------ |
| NOV68d | ------------------------------------------------------------ |
| NOV68e | ------------------------------------------------------------ |
| NOV68f | ------------------------------------------------------------ |
| NOV68g | RLCNNPEPKFGGKYCTGERKRYRLCNVHPCRSEAPTFRQMQCSEFDTVPYKNELYHWFPI |
| NOV68h | ------------------------------------------------------------ |
| NOV68i | ------------------------------------------------------------ |
| NOV68j | ------------------------------------------------------------ |
| NOV68k | ------------------------------------------------------------ |
| NOV68l | ------------------------------------------------------------ |
| NOV68m | ------------------------------------------------------------ |
| NOV68a | SISKERWVETLVVADTKMIEYHGSENVESYILTIMNMVTGLFHNPSIGNAIHIVVVRLIL |
| NOV68b | ------------------------------------------------------------ |
| NOV68c | ------------------------------------------------------------ |
| NOV68d | ------------------------------------------------------------ |
| NOV68e | ------------------------------------------------------------ |
| NOV68f | ------------------------------------------------------------ |
| NOV68g | FNPAHPCELYCRPIDGQFSEKMLDAVIDGTPCFEGGNSRNVCINGICKMVGCDYEIDSNA |
| NOV68h | ------------------------------------------------------------ |
| NOV68i | ------------------------------------------------------------ |
| NOV68j | ------------------------------------------------------------ |
| NOV68k | ------------------------------------------------------------ |
| NOV68l | ------------------------------------------------------------ |
| NOV68m | ------------------------------------------------------------ |
| NOV68a | LEEEEQGLKIVHHAEKTLSSFCKWQKSINPKSDLNPVHHDVAVLLTRKDICAGFNRPCET |
| NOV68b | ------------------------------------------------------------ |
| NOV68c | ------------------------------------------------------------ |
| NOV68d | ------------------------------------------------------------ |
| NOV68e | -----------------------------------------RSYGRQPQPGPVRFPDRRQ |
| NOV68f | -----------------------------------------RSYGRQPQPGPVRFPDRRQ |
| NOV68g | TEDRCGVCLGDGSSCQTARKMFKQKEGSGYVDIGLIPKGARDIRVHEIEGAGNFLAIRSE |
| NOV68h | ------------------------------------------------------------ |
| NOV68i | ------------------------------------------------------------ |
| NOV68j | ------------------------------------------------------------ |
| NOV68k | -------------------------------------------YGRQPQPGPVRFPDRRQ |
| NOV68l | ------------------------------------------------------------ |
| NOV68m | ------------------------------------------------------------ |
| NOV68a | LGLSHLSGMCQPHRSCNINEDSGLPLAFTIAHELGHSFGIQHDGKENDCEPVGRHPYIMS |
| NOV68b | ------------------------------------------------------------ |
| NOV68c | ------------------------------------------------------------ |
| NOV68d | ------------------------------------------------------------ |
| NOV68e | EHFIKGLPEYHVVGPVRVDASGHFLSYGLHYPITSSRRKRDLDGSEDWVYYRISHEEKDL |
| NOV68f | ENFIKGLPEYHVVGPVRVDASGHFLSYGLHYPITSSRRKRDLDGSEDWVYYRISHEEKDL |
| NOV68g | DPEKYYLNGGFIIQWNGNYKLAGTVFQYDRKGDLEKLMATGPTNESVWIQLLFQVTNPGI |
| NOV68h | ------------------------------------------------------------ |
| NOV68i | ------------------------------------------------------------ |
| NOV68j | ------------------------------------------------------------ |
| NOV68k | EHFIKGLPEYHVVGPVRVDASGHFLSYGLHYPITSSRRKRDLDGSEDWVYYRISHEEKDL |
| NOV68l | ------------------------------------------------------------ |
| NOV68m | ------------------------------------------------------------ |
| NOV68a | RQLQYDPTPLTWSKCSEEYITRFLDRGWGFCLDDIPKKKGLKSKVIAPGVIYDVHHQCQL |
| NOV68b | KLNLTVNQGFLSNSYIMEKRYGNLSHVKMMASSAPLCHLSGTVLQQGTRVGMAALSACHG |
| NOV68c | KLNLTVNQGFLSNSYIMEKRYGNLSHVKMMASSAPLCHLSGTVLQQGTRVGTAALSACHG |
| NOV68d | KLNLTVNQGFLSNSYIMEKRYGNLSHVKMMASSAPLCHLSGTVLQQGTRVGTAALSACHG |
| NOV68e | FFNLTVNQGFLSNSYIMEKRYGNLSHVKMMASSAPLCHLNGTVLQQGTRVGTAALSACHG |
| NOV68f | FFNLTVNQGFLSNSYIMEKRYGNLSHVKMMASSAPLCHLSGTVLQQGTRVGTAALSACHG |
| NOV68g | KYEYTIQKDGLDNDVEQQMYFWQYGHWTECSVTCGTGIRRQTAHCIKKGRGMVKATFCDP |
| NOV68h | --NLTVNQGFLSNSYIMEKRYGNLSHVKMMASSAPLCHLSGTVLQQGTRVGMAALSACHG |
| NOV68i | --NLTVNQGFLSNSYIMEKRYGNLSHVKMMASSAPLCHLSGTVLQQGTRVGTAALSACHG |
| NOV68j | -------------------------------------------------------HQCQL |
| NOV68k | FFNLTVNQGFLSNSYIMEKRYGNLSHVKMMASSAPLCHLSGTVLQQGTRVGTAALSACHG |
| NOV68l | KLNLTVNQGFLSNSYIMEKRYGNLSHVKMMASSAPLCHLSGTVLQQGTRVGTAALSACHG |
| NOV68m | -----------------------------------------------------KLHQCQL |
| NOV68a | QYGPNATFCQEVENVCQTLWCSVKGFCRSKLDAAA------------------------- |
| NOV68b | LTGFFQLPHGDFFIEPVKKHPLVEGGYHPHIVYRR------------------------- |
| NOV68c | LTGFFQLPHGDFFIEPVKKHPLVEGGYHPHIVYRR------------------------- |
| NOV68d | LTGFFQLPHGDFFIEPVKKHPLVEGGYHPHIVYRR------------------------- |
| NOV68e | LTGFFQLPHGDFFIEPVKKHPLVEGGYHPHIVYRR------------------------- |
| NOV68f | LTGFFQLPHGDFFIEPVKKHPLVEGGYHPHIVYRR------------------------- |
| NOV68g | ETQPNGRQKKCHEKACPPRWWAGEWEACSATCGPHGEKKRTVLCIQTMVSDEQALPPTDC |
| NOV68h | LTGFFQLPHGDFFIEPVKKHPLVEGGYHPHIVYRR------------------------- |
| NOV68i | LTGFFQLPHGDFFIEPVKKHPLVEGGYHPHIVYRR------------------------- |
| NOV68j | QYGPNATFCQEVENVCQTLWCSVKGFCRSKLDAAA------------------------- |
| NOV68k | LTGFFQLPHGDFFIEPVKKHPLVEGGYHPHIVYRR------------------------- |
| NOV68l | LTGFFQLPHGDFFIEPVKKHPLVEGGYHPHIVYRR------------------------- |
| NOV68m | QYGPNATFCQEVENVCQTLWCSVKGFCRSKLDAAA------------------------- |
| NOV68a | --------------------------------------------------DGTQCGEKK- |
| NOV68b | --------------------------------------------------QKVPETKEP- |
| NOV68c | --------------------------------------------------QKVPETKEP- |
| NOV68d | --------------------------------------------------QKVPETKEP- |
| NOV68e | --------------------------------------------------QKVPETKEP- |
| NOV68f | --------------------------------------------------QKVPETKEP- |
| NOV68g | QHLLKPKTLLSCNRDILCPSDWTVGNWSECSVSCGGGVRIRSVTCAKNHDEPCDVTRKPN |
| NOV68h | --------------------------------------------------QKVPETKEP- |
| NOV68i | --------------------------------------------------QKVPETKEP- |
| NOV68j | --------------------------------------------------DGTQCGEKK- |
| NOV68k | --------------------------------------------------QKVPETKEP- |
| NOV68l | --------------------------------------------------QKVPETKEP- |
| NOV68m | --------------------------------------------------DGTQCGEKK- |
| NOV68a | ---WCMAGKC-------------------------------------------------- |
| NOV68b | ---TCGLKDS-------------------------------------------------- |
| NOV68c | ---TCGLKDS-------------------------------------------------- |
| NOV68d | ---TCGLKDS-------------------------------------------------- |
| NOV68e | ---TCGLKDS-------------------------------------------------- |
| NOV68f | ---TCGLKDS-------------------------------------------------- |
| NOV68g | SRALCGLQQCPSSRRVLKPNKGTISNGKNPPTLKPVPPPTSRPRMLTTPTGPESMSTSTP |
| NOV68h | ---TCGLKDS-------------------------------------------------- |
| NOV68i | ---TCGLKDS-------------------------------------------------- |
| NOV68j | ---WCMAGKC-------------------------------------------------- |
| NOV68k | ---TCGLKDS-------------------------------------------------- |
| NOV68l | ---TCGLKDS-------------------------------------------------- |
| NOV68m | ---WCMAGKC-------------------------------------------------- |
| NOV68a | -------ITVGKKPESIPGGWGRWSPWSHCSRTCGAGVQSAERLCNNPEPKFGGKYCT-- |
| NOV68b | -------VNISQKQELWREKWERHNLPSRSLSRRSISKERWVETLVVADTKMIEYHGS-- |
| NOV68c | -------VNISQKQELWREKWERHNLPSRSLSRRSISKERWVETLVVADTKMIEYHGS-- |
| NOV68d | -------VNISQKQELWREKWERHNLPSRSLSRRSISKERWVETLVVADTKMIEYHGS-- |
| NOV68e | -------VNISQKQELWREKWERHNLPSRSLSRRSISKERWVETLVVADTKMIEYHGS-- |
| NOV68f | -------VNISQKQELWREKWERHNLPSRSLSRRSISKERWVETLVVADTKMIEYHGS-- |
| NOV68g | AISSPSPTTASKEGDLGGKQWQDSSTQPELSSRYLISTGSTSQPILTSQSLSIQPSEENV |
| NOV68h | -------VNISQKQELWREKWERHNLPSRSLSRRSISKERWVETLVVADTKMIEYHGS-- |
| NOV68i | -------VNISQKQELWREKWERHNLPSRSLSRRSISKERWVETLVVADTKMIEYHGS-- |
| NOV68j | -------ITVGKKPESIPGGCGRWSPWSHCS----------------------------- |
| NOV68k | -------VNISQKQELWREKWERHNLPSRSLSRRSISKERWVETLVVADTKMIEYHGS-- |
| NOV68l | -------VNISQKQELWREKWERHNLPSRSLSRRSISKERWVETLVVADTKMIEYHGS-- |
| NOV68m | -------ITVGKKPESIPGGWGRWSPWSHCSLE--------------------------- |
| NOV68a | --------------------GERKRYRLCNVHPCRSEAPTFRQMQCSEFDTVPYKNELYH |
| NOV68b | --------------------ENVESCILTIMNMVTGLFHNPSIGNAIHIVVVRLILLEEE |
| NOV68c | --------------------ENVESYILTIMNMVTGLFHNPSIGNAIHIVVVRLILLEEE |
| NOV68d | --------------------ENVESYILTIMNMVTGLFHNPSIGNAIHIVVVRLILLEEE |
| NOV68e | --------------------ENVESYILTIMNMVTGLFHNPSIGNAIHIVVVRLILLEEE |
| NOV68f | --------------------ENVESYILTIMNMVTGLFHNPSIGNAIHIVVVRLILLEEE |
| NOV68g | SSSDTGPTSEGGLVATTTSGSGLSSSRNPITWPVTPFYNTLTKGPEMEIHSGSGEEREQP |
| NOV68h | --------------------ENVESCILTIMNMVTGLFMNPSIGNAIHIVVVRLILLEEE |
| NOV68i | --------------------ENVESYILTIMNMITGLFHNPSIGNAIHIVVVRLILLEEE |
| NOV68j | ------------------------------------------------------------ |
| NOV68k | --------------------ENVESYILTIMNMVTGLFHNPSIGNAIHIVVVRLILLEEE |
| NOV68l | --------------------ENVESYILTIMNNVTGLFHNPSIGNAIHIVVVRLILLEEE |
| NOV68m | ------------------------------------------------------------ |
| NOV68a | WFP--------------------------------------------------------- |
| NOV68b | EQG--------------------------------------------------------- |
| NOV68c | EQG--------------------------------------------------------- |
| NOV68d | EQG--------------------------------------------------------- |
| NOV68e | EQG--------------------------------------------------------- |
| NOV68f | EQG--------------------------------------------------------- |
| NOV68g | EDKDESNPVIWTKIRVPGNDAPVESTEMPLAPPLTPDLSRESWWPPFSTVMEGLLPSQRP |
| NOV68h | EQG--------------------------------------------------------- |
| NOV68i | EQG--------------------------------------------------------- |
| NOV68j | ------------------------------------------------------------ |
| NOV68k | EQG--------------------------------------------------------- |
| NOV68l | EQG--------------------------------------------------------- |
| NOV68m | ------------------------------------------------------------ |
| NOV68a | ------------------------------------------IFNPAHPCELYCR----- |
| NOV68b | ------------------------------------------LKIVHHAEKTLSS----- |
| NOV68c | ------------------------------------------LKIVNHAEKTLSS----- |
| NOV68d | ------------------------------------------LKIVHHAEKTLSS----- |
| NOV68e | ------------------------------------------LKIVHHAEKTLSS----- |
| NOV68f | ------------------------------------------LKIVHHAEKTLSS----- |
| NOV68g | TTSETGTPRVEGMVTEKPANTLLPLGGDHQPEPSGKTANRNHLKLPNNMNQTKSSEPVLT |
| NOV68h | ------------------------------------------LKIVHHAEKTLSS----- |
| NOV68i | ------------------------------------------LKIVHHAEKTLSS----- |
| NOV68j | ------------------------------------------------------------ |
| NOV68k | ------------------------------------------LKIVHHAEKTLSS----- |
| NOV68l | ------------------------------------------LKIVHHAEKTLSS----- |
| NOV68m | ------------------------------------------------------------ |
| NOV68a | ----------------------------------------------------------PIDGQFSEKMLD |
| NOV68b | ----------------------------------------------------------FCKWQKSINPKS |
| NOV68c | ----------------------------------------------------------FCKWQKSINPKS |
| NOV68d | ----------------------------------------------------------FCKWQKSINPKS |
| NOV68e | ----------------------------------------------------------FCKWQKSINPKS |
| NOV68f | ----------------------------------------------------------FCKWQKSINPKS |
| NOV68g | EEDATSLITEGFLLNASNYKQLTNGYGSAHWIVGNWSECSTTCGLGAYWRRVECSTQMDS |
| NOV68h | ----------------------------------------------------------FCKWQKSINPKS |
| NOV68i | ----------------------------------------------------------FCKWQKSINPKS |
| NOV68j | ------------------------------------------------------------ |
| NOV68k | ----------------------------------------------------------FCKWQKSINPKS |
| NOV68l | ----------------------------------------------------------FCKWQKSINPKS |
| NOV68m | ------------------------------------------------------------ |
| NOV68a | AVIDGTPCFEGGNSRNVCINGICKMVGCDYEIDSNATEDRCGVCLGDG-SSCQTVRKMFK |
| NOV68b | DLNPVHHDVAVLLTRKDICAGFNRPCETLGLSHLSGMCQPHRSCNINE-DSGLPLAFTIA |
| NOV68c | DLNPVHHDVAVLLTRKDICAGFNRPCETLGLSHLSGMCQPHRSCNINE-DSGLPLAFTIA |
| NOV68d | DLNPVHHDVAVLLTRKDICAGFNRPCETLGLSHLSGNCQPHRSCNINE-DSGLPLAFTIA |
| NOV68e | DLNPVHHDVAVLLTRKDICAGFNRPCETLGLSHLSGMCQPHRSCNINE-DSGLPLAFTIA |
| NOV68f | DLNPVHHDVAVLLTRKDICAGFNRPCETLGLSHLSGMCQPHRSCNINE-DSGLPLAFTIA |
| NOV68g | DCAAIQRPDPAKRCHLRPCAGWKVGNWSKCSRNCSGGFKIREIQCVDSRDHRNLRPFHCQ |
| NOV68h | DLNPVHHDVAVLLTRKDICAGFNRPCETLGLSHLSGMCQPHRSCNINE-DSGLPLAFTIA |
| NOV68i | DLNPVHHDVAVLLTRKDICAGFNRPCETLGLSHLSGMCQPHRSCNINE-DSGLPLAFTIA |
| NOV68j | ------------------------------------------------------------ |
| NOV68k | DLNPVHHDVAVLLTRKDICAGFNRPCETLGLSHLSGMCQPHRSCNINE-DSGLPLAFTIA |
| NOV68l | DLNPVHHDVAVLLTRKDICAGFNRPCETLGLSHLSGMCQPHRSCNINE-DSGLPLAFTIA |
| NOV68m | ------------------------------------------------------------ |
| NOV68a | QKEGSGYVDIGLIPKGARDIRVMEIEGAGNFLAIRSE-----------------DPEKYY |
| NOV68b | HELGHSFGIQHDGKENDCEPVGRHPYIMSRQLQYDPT-----------------PLTWSK |
| NOV68c | HELGHSFGIQHDGKENDCEPVGRHPYIMSRQLQYDPT-----------------PLTWSK |
| NOV68d | HELGHSFGIQHDGKENDCEPVGRHPYIMSRQLQYDPT-----------------PLTWSK |
| NOV68e | HELGHSFGIQHDGKENDCEPVGRHPYIMSRQLQYDPT-----------------PLTWSK |
| NOV68f | HELGHSFGIQHDGKENDCEPVGRHPYIMSRQLQYDPT-----------------PLTWSK |
| NOV68g | FLAGIPPPLSMSCNPEPCEAWQVEPWSQCSRSCGGGVOERGVFCPGGLCDWTKRPTSThS |
| NOV68h | HELGHSFGIQHDGKENDCEPVGRHPYIMSRQLQYDPT-----------------PLTWSK |
| NOV68i | HELGHSFGIQHDGKENDCEPVGRHPYIMSRQLQYDPT-----------------PLTWSK |
| NOV68j | ------------------------------------------------------------ |
| NOV68k | HELGHSFGIQHDGKENDCEPVGRHPYIMSRQLQYDPT-----------------PLTWSK |
| NOV68l | HELGHSFGIQHDGKENDCEPVGRHPYIMSRQLQYDPT-----------------PLTWSK |
| NOV68m | ------------------------------------------------------------ |
| NOV68a | LNGGFIIQWNGNYKLAGTVFQYDRKGDLEKLMATGPTNESVWIQLLFQVTNPGIKYEYTI |
| NOV68b | CSEEYITRFLDRGWGFCLDDIPLE------------------------------------ |
| NOV68c | CSEEYITRFLDRGWGFCLDDIPLE------------------------------------ |
| NOV68d | CSEEYITRFLDRGWGFCLDDIPLE------------------------------------ |
| NOV68e | CSEEYITRFLDRGWGFCLDDIPKKKGLKSKVIAPGVIYDVHHQCQLQYGPNATLCQEVEN |
| NOV68f | CSEEYITRFLDRGWGFCLDDIPKKKGLKSKVIAPGVIYDVHHQCQLQYGPNATLCQEVEN |
| NOV68g | CNEHLCCHWATGNWDLCSTSCGGGFQKRTVQCVPSEGNKTEDQDQCLCDHKPRPPEFKKC |
| NOV68h | CSEEYITRFLDRGWGFCLDDIP-------------------------------------- |
| NOV68i | CSEEYITRFLDRGWGFCLDDIP-------------------------------------- |
| NOV68j | ------------------------------------------------------------ |
| NOV68k | CSEEYITRFLDRGWGFCLDDIPKKKGLKSKVIAPGVIYDVHHQCQLQYGPNATFCQEVEN |
| NOV68l | CSEEYITRFLDRGWGFCLDDIPLE------------------------------------ |
| NOV68m | ------------------------------------------------------------ |
| NOV68a | QKDGLDNDVEQMYFWQYGHWTECSVTCGTGIRRQTAHCIKKGRGMVKATFCDPETQPNGR |
| NOV68b | ------------------------------------------------------------ |
| NOV68c | ------------------------------------------------------------ |
| NOV68d | ------------------------------------------------------------ |
| NOV68e | VCQTLWCSVKGFCRSKLDAAADGTQCGEKKWCMAGKCITVGKKPESIPGGWGRWSPWSHC |
| NOV68f | VCQTLWCSVKGFCRSKLDAAADGTQCGEKKWCMAGKCITVGKKPESIPGGWGRWSPWSHC |
| NOV68g | NQQACKKSADLLCTKDKLSASFCQTLKAMKKCSVPTVRAECCFSCPQTHITHTQRQRRQR |
| NOV68h | ------------------------------------------------------------ |
| NOV68i | ------------------------------------------------------------ |
| NOV68j | ------------------------------------------------------------ |
| NOV68k | VCQTLWCSVKGFCRSKLDAAADGTQCGEKKWCMAGKCITVGKKPESIPGGWGRWSPWSHC |
| NOV68l | ------------------------------------------------------------ |
| NOV68m | ------------------------------------------------------------ |
| NOV68a | QKKCHEKACPPRWWAGEWEACSATCGPHGEKKRTVLCIQTMVSDEQALPPTDCQHLLKPK |
| NOV68b | ------------------------------------------------------------ |
| NOV68c | ------------------------------------------------------------ |
| NOV68d | ------------------------------------------------------------ |
| NOV68e | SRTCGAGVQSAERLCNNPEPKFGGKYCTGERKRYRLCNVHPCRSEAPTFRQMQCSEFDTV |
| NOV68f | SRTCGAGVQSAERLCNNPEPKFGGKYCTGERKRYRLCNVHPCRSEAPTFRQMQCSEFDTV |
| NOV68g | LLQKSKELLE-------------------------------------------------- |
| NOV68h | ------------------------------------------------------------ |
| NOV68i | ------------------------------------------------------------ |
| NOV68j | ------------------------------------------------------------ |
| NOV68k | SRTCGAGVQSAERLCNNPEPKFGGKYCTGERKRYRLCNVHPCRSEAPTFRQMQCSEFDTV |
| NOV68l | ------------------------------------------------------------ |
| NOV68m | ------------------------------------------------------------ |
| NOV68a | TLLSCNRDILCPSDWTVGNWSECSVSCGGGVRIRSVTCAKNHDEPCDVTRKPNSRALCGL |
| NOV68b | ------------------------------------------------------------ |
| NOV68c | ------------------------------------------------------------ |
| NOV68d | ------------------------------------------------------------ |
| NOV68e | PYKNELYHWFPIFNPAHPCELYCRPIDGQFSEKMLDAVIDGTPCFEGGNSRNVCINGICK |
| NOV68f | PYKNELYHWFPIFNPAHPCELYCRPIDGQFSEKMLDAVIDGTPCFEGGNSRNVCINGICK |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | PYKNELYHWFPIFNPAHPCELYCRPIDGQFSEKMLDAVIDGTPCFEGGNSRNVCINGICK |
| NOV68l | ------------------------------------------------------------ |
| NOV68m | ------------------------------------------------------------ |
| NOV68a | QQCPSSRRVLKPNKGTISNGKNPPTLKPVPPPTSRPRMLTTPTGPESMSTSTPAISSPSP |
| NOV68b | ------------------------------------------------------------ |
| NOV68c | ------------------------------------------------------------ |
| NOV68d | ------------------------------------------------------------ |
| NOV68e | MVGCDYEIDSNATEDRCGVCLGDGSSCQTVRKMFKQKEGSGYVDIGLIPKGARDIRVMEI |
| NOV68f | MVGCDYEIDSNATEDRCGVCLGDGSSCQTVRKMFKQKEGSGYVDIGLIPKGARDIRVMEI |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | MVGCDYEIDSNATEDRCGVCLGDGSSCQTVRKMFKQKEGSGYVDIGLIPKGARDIRVMEI |
| NOV68l | ------------------------------------------------------------ |
| NOV68m | ------------------------------------------------------------ |
| NOV68a | TTASKEGDLGGKQWQDSSTQPELSSRYLISTGSTSQPILTSQSLSIQPSEENVSSSDTGP |
| NOV68b | ------------------------------------------------------------ |
| NOV68c | ------------------------------------------------------------ |
| NOV68d | ------------------------------------------------------------ |
| NOV68e | EGAGNFLAIRSEDPEKYYLNGGFIIQWNGNYKLAGTVFQYDRKGDLEKLMATGPTNESVW |
| NOV68f | EGAGNFLAIRSEDPEKYYLNGGFIIQWNGNYKLAGTVFQYDRKGDLEKLMATGPTNESVW |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | EGAGNFLAIRSEDPEKYYLNGGFIIQWNGNYKLAGTVFQYDRKGDLEKLMATGPTNESVW |
| NOV68l | ------------------------------------------------------------ |
| NOV68m | ------------------------------------------------------------ |
| NOV68a | TSEGGLVATTTSGSGLSSSRNPITWPVTPFYNTLTKGPEMEIHSGSGEEREQPEDKDESN |
| NOV68b | ------------------------------------------------------------ |
| NOV68c | ------------------------------------------------------------ |
| NOV68d | ------------------------------------------------------------ |
| NOV68e | IQLLFQVTNPGIKYEYTIQKDGLDNDVEQQMYFWQYGHWTECSVTCGTGIRRQTAHCIKK |
| NOV68f | IQLLFQVTNPGIKYEYTIQKDGLDNDVEQQMYFWQYGHWTECSVTCGTGIRRQTAHCIKK |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | IQLLFQVTNPGIKYEYTIQKDGLDNDVEQQMYFWQYGHWTECSVTCGTGIRRQTAHCIKK |
| NOV68l | ------------------------------------------------------------ |
| NOV68m | ------------------------------------------------------------ |
| NOV68a | PVIWTKIRVPGNDAPVESTEMPLAPPLTPDLSRESWWPPFSTVMEGLLPSQRPTTSETGT |
| NOV68b | ------------------------------------------------------------ |
| NOV68c | ------------------------------------------------------------ |
| NOV68d | ------------------------------------------------------------ |
| NOV68e | GRGMVKATFCDPETQPNGRQKKCHEKACPPRWWAGEWEACSATCGPHGEKKRTVLCIQTM |
| NOV68f | GRGMVKATFCDPETQPNGRQKKCHEKACPPRWWAGEWEACSATCGPHGEKKRTVLCIQTM |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | GRGMVKATFCDPETQPNGRQKKCHEKACPPRWWAGEWEACSATCGPHGEKKRTVLCIQTM |
| NOV68l | ------------------------------------------------------------ |
| NOV68m | ------------------------------------------------------------ |
| NOV68a | PRVEGMVTEKPANTLLPLGGDHQPEPSGKTANRNHLKLPNNMNQTKSSEPVLTEEDATSL |
| NOV68b | ------------------------------------------------------------ |
| NOV68c | ------------------------------------------------------------ |
| NOV68d | ------------------------------------------------------------ |
| NOV68e | VSDEQALPPTDCQHLLKPKTLLSCNRDILCPSDWTVGNWSECSVSCGGGVRIRSVTCAKN |
| NOV68f | VSDEQALPPTDCQHLLKPKTLLSCNRDILCPSDWTVGNWSECSVSCGGGVRIRSVTCAKN |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | VSDEQALPPTDCQHLLKPKTLLSCNRDILCPSDWTVGNWSECSVSCGGGVRIRSVTCAKN |
| NOV68l | ------------------------------------------------------------ |
| NOV68m | ------------------------------------------------------------ |
| NOV68a | ITEGFLLNASNYKQLTNGHGSAHWIVGNWSECSTTCGLGAYWKRVECTTQMDSDCAAIQR |
| NOV68b | ------------------------------------------------------------ |
| NOV68c | ------------------------------------------------------------ |
| NOV68d | ------------------------------------------------------------ |
| NOV68e | HDEPCDVTRKPNSRALCGLQQCPSSRRVLKPNKGTISNGKNPPTLKPVPPPTSRPRMLTT |
| NOV68f | HDEPCDVTRKPNSRALCGLQQCPSSRRVLKPNKGTISNGKNPPTLKPVPPPTSRPRMLTT |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | HDEPCDVTRKPNSRALCGLQQCPSSRRVLKPNKGTISNGKNPPTLKPVPPPTSRPRMLTT |
| NOV68l | ------------------------------------------------------------ |
| NOV68m | ------------------------------------------------------------ |
| NOV68a | PDPAKRCHLRPCAGWKVGNWSKCSRNCSGGFKIREIQCVDSRDHRNLRPFHCQFLAGIPP |
| NOV68b | ------------------------------------------------------------ |
| NOV68c | ------------------------------------------------------------ |
| NOV68d | ------------------------------------------------------------ |
| NOV68e | PTGPESMSTSTPAISSPSPTTASKEGDLGGKQWQDSSTQPELSSRTLISTGSTSQPILTS |
| NOV68f | PTGPESMSTSTPAISSPSPTTASKEGDLGGKQWQDSSTQPELSSRYLISTGSTSQPILTS |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | PTGPESMSTSTPAISSPSPTTASKEGDLGGKQWQDSSTQPELSSRYLISTGSTSQPILTS |
| NOV68l | ------------------------------------------------------------ |
| NOV68m | ------------------------------------------------------------ |
| NOV68a | PLSMSCNPEPCEAWQVEPWSQCSRSCGGGVQERGVFCPGGLCDWTKRPTSTMSCNEHLCC |
| NOV68b | ------------------------------------------------------------ |
| NOV68c | ------------------------------------------------------------ |
| NOV68d | ------------------------------------------------------------ |
| NOV68e | QSLSIQPSEENVSSSDTGPTSEGGLVATTTSGSGLSSSRNPITWPVTPFYNTLTKGPEME |
| NOV68f | QSLSIQPSEENVSSSDTGPTSEGGLVATTTSGSGLSSSRNPITWPVTPFYNTLTKGPEME |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | QSLSIQPSEENVSSSDTGPTSEGGLVATTTSGSGLSSSRNPITWPVTPFYNTLTKGPEME |
| NOV68l | ------------------------------------------------------------ |
| NOV68m | ------------------------------------------------------------ |
| NOV68a | HWATGNWDLCSTSCGGGFQKRIVQCVPSEGNKTEDQDQCLCDHKPRPPEFKKCNQQACKK |
| NOV68b | ------------------------------------------------------------ |
| NOV68c | ------------------------------------------------------------ |
| NOV68d | ------------------------------------------------------------ |
| NOV68e | IHSGSGEEREQPEDKDESNPVIWTKIRVPGNDAPVESTEMPLAPPLTPDLSRESWWPPFS |
| NOV68f | IHSGSGEEREQPEDKDESNPVIWTKIRVPGNDAPVESTEMPLAPPLTPDLSRESWWPPFS |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | IHSGSGEEREQPEDKDESNPVIWTKIRVPGNDAPVESTEMPLAPPLTPDLSRESWWPPFS |
| NOV68l | ------------------------------------------------------------ |
| NOV68m | ------------------------------------------------------------ |
| NOV68a | SADLLCTKDKLSASFCQTLKAMKKCSVPTVRAECCFSCPQTHITHTQRQRRQRLLQKSKE |
| NOV68b | ------------------------------------------------------------ |
| NOV68c | ------------------------------------------------------------ |
| NOV68d | ------------------------------------------------------------ |
| NOV68e | TVMEGLLPSQRPTTSETGTPRVEGMVTEKPANTLLPLGGDHQPEPSGKTANRNHLKLPNN |
| NOV68f | TVMEGLLPSQRPTTSETGTPRVEGMVTEKPANTLLPLGGDHQPEPSGKTANRNHLKLPNN |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | TVMEGLLPSQRPTTSETGTPRVEGMVTEKPANTLLPLGGDHQPEPSGKTANRNHLKLPNN |
| NOV68l | ------------------------------------------------------------ |
| NOV68m | ------------------------------------------------------------ |
| NOV68a | L----------------------------------------------------------- |
| NOV68b | ------------------------------------------------------------ |
| NOV68c | ------------------------------------------------------------ |
| NOV68d | ------------------------------------------------------------ |
| NOV68e | MNQTKSSEPVLTEEDATSLITEGFLLNASNYKQLTNGHGSAHWIVGNWSECSTTCGLGAY |
| NOV68f | MNQTKSSEPVLTEEDATSLITEGFLLNASNYKQLTNGHGSAHWIVGNWSECSTTCGLGAY |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | MNQTKSSEPVLTEEDATSLITEGFLLNASNYKQLTNGHGSAHWIVGNWSECSTTCGLGAY |
| NOV68l | ------------------------------------------------------------ |
| NOV68m | ------------------------------------------------------------ |
| NOV68a | ------------------------------------------------------------ |
| NOV68b | ------------------------------------------------------------ |
| NOV68c | ------------------------------------------------------------ |
| NOV68d | ------------------------------------------------------------ |
| NOV68e | WRRVECSTQMDSDCAAIQRPDPAKRCHLRPCAGWKVGNWSKCSRNCSGGFKIREIQCVDS |
| NOV68f | WRRVECSTQMDSDCAAIQRPDPAKRCHLRPCAGWKVGNWSKCSRNCSGGFKIREIQCVDS |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | WRRVECSTQMDSDCAAIQRPDPAKRCHLRPCAGWKVGNWSKCSRNCSGGFKIREIQCVDS |
| NOV68l | ------------------------------------------------------------ |
| NOV68m | ------------------------------------------------------------ |
| NOV68a | ------------------------------------------------------------ |
| NOV68b | ------------------------------------------------------------ |
| NOV68c | ------------------------------------------------------------ |
| NOV68d | ------------------------------------------------------------ |
| NOV68e | RDHRNLRPFHCQFLAGIPPPLSMSCNPEPCEAWQVEPWSQCSRSCGGGVQERGVFCPGGL |
| NOV68f | RDHRNLRPFHCQFLAGIPPPLSMSCNPEPCEAWQVEPWSQCSRSCGGGVQERGVFCPGGL |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | RDHRNLRPFHCQFLAGIPPPLSMSCNPEPCEAWQVEPWSQCSRSCGGGVQERGVFCPGGL |
| NOV68l | ------------------------------------------------------------ |
| NOV68m | ------------------------------------------------------------ |
| NOV68a | ------------------------------------------------------------ |
| NOV68b | ------------------------------------------------------------ |
| NOV68c | ------------------------------------------------------------ |
| NOV68d | ------------------------------------------------------------ |
| NOV68e | CDWTKRPTSTMSCNEHLCCHWATGNWDLCSTSCGGGFQKRTVQCVPSEGNKTEDQDQCLC |
| NOV68f | CDWTKRPTSTMSCNEHLCCHWATGNWDLCSTSCGGGFQKRTVQCVPSEGNKTEDQDQCLC |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | CDWTKRPTSTMSCNEHLCCHWATGNWDLCSTSCGGGFQKRTVQCVPSEGNKTEDQDQCLC |
| NOV68l | ------------------------------------------------------------ |
| NOV68m | ------------------------------------------------------------ |
| NOV68a | ------------------------------------------------------------ |
| NOV68b | ------------------------------------------------------------ |
| NOV68c | ------------------------------------------------------------ |
| NOV68d | ------------------------------------------------------------ |
| NOV68e | DHKPRPPEFKKCNQQACKKSADLLCTKDKLSASFCQTLKAMKKCSVPTVRAECCFSCPQT |
| NOV68f | DHKPRPPEFKKCNQQACKKSADLLCTKDKLSASFCQTLKAMKKCSVPTVRAECCFSCPQT |
| NOV68g | ------------------------------------------------------------ |
| NOV68h | ------------------------------------------------------------ |
| NOV68i | ------------------------------------------------------------ |
| NOV68j | ------------------------------------------------------------ |
| NOV68k | DHKPRPPEFKKCNQQACKKSADLLCTKDKLSASFCQTLKAMKKCSVPTVRAECCFSCPQT |
| NOV68l | ------------------------------------------------------------ |
| NOV68m | ------------------------------------------------------------ |
| NOV68a | ------------------------- |
| NOV68b | ------------------------- |
| NOV68c | ------------------------- |
| NOV68d | ------------------------- |
| NOV68e | HITHTQRQRRQRLLQKSKELLEEFF |
| NOV68f | HITHTQRQRRQRLLQKSKGLLEEFF |
| NOV68g | ------------------------- |
| NOV68h | ------------------------- |
| NOV68i | ------------------------- |
| NOV68j | ------------------------- |
| NOV68k | HITHTQRQRRQRLLQKSKGLLEEFF |
| NOV68l | ------------------------- |
| NOV68m | ------------------------- |
| NOV68a | (SEQ ID NO: 1006) |
| NOV68b | (SEQ ID NO: 1008) |
| NOV68c | (SEQ ID NO: 1010) |
| NOV68d | (SEQ ID NO: 1012) |
| NOV68e | (SEQ ID NO: 1014) |
| NOV68f | (SEQ ID NO: 1016) |
| NOV68g | (SEQ ID NO: 1018) |
| NOV68h | (SEQ ID NO: 1020) |
| NOV68i | (SEQ ID NO: 1022) |
| NOV68j | (SEQ ID NO: 1024) |
| NOV68k | (SEQ ID NO: 1026) |
| NOV68l | (SEQ ID NO: 1028) |
| NOV68m | (SEQ ID NO: 1030) |

Further analysis of the NOV68a protein yielded the following properties shown in Table 68C.

TABLE 68C
Protein Sequence Properties NOV68a

| | |
|---|---|
| SignalP analysis: | Cleavage site between residues 26 and 27 |

PSORT II analysis:

| | |
|---|---|
| PSG: | a new signal peptide prediction method |
| | N-region: length 6; pos.chg 1; neg.chg 0 |
| | H-region: length 21; peak value 6.23 |
| | PSG score: 1.83 |
| GvH: | von Heijne's method for signal seq. recognition |
| | GvH score (threshold: −2.1): −3.11 |
| | possible cleavage site: between 27 and 28 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 2 |
| | Number of TMS(s) for threshold 0.5: 0 |
| | PERIPHERAL Likelihood = 4.98 (at 267) |
| | ALOM score: −1.54 (number of TMSs: 0) |
| MTOP: | Prediction of membrane topology (Hartmann et al.) |
| | Center position for calculation: 6 |
| | Charge difference: 1.0 C( 3.0) - N( 2.0) |
| | C > N: C-terminal side will be inside |

>>>Caution: Inconsistent mtop result with signal peptide
MITDISC: discrimination of mitochondrial targeting seq

| | | | |
|---|---|---|---|
| R content: | 3 | Hyd Moment (75): | 8.02 |
| Hyd Moment (95): | 8.85 | G content: | 3 |
| D/E content: | 1 | S/T content: | 2 |
| Score: −2.50 | | | |

Gavel: prediction of cleavage sites for mitochondrial preseq

R-2 motif at 46 VRF|PD

NUCDISC: discrimination of nuclear localization signals

pat4: RRKR (5) at 79
pat4: PKKK (4) at 456
pat7: PKKKGLK (5) at 456
pat7: PNGRQKK (3) at 869
pat7: PHGEKKR (3) at 899
bipartite: none
content of basic residues: 11.5%
NLS Score: 1.04

KDEL: ER retention motif in the C-terminus: none
ER Membrane Retention Signals:

KKXX-like motif in the C-terminus: KSKE

SKL: peroxisomal targeting signal in the C-terminus: none
PTS2: 2nd peroxisomal targeting signal: none
VAC: possible vacuolar targeting motif: found

KLPN at 1269

RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: nuclear
Reliability: 89

COIL: Lupas's algorithm to detect coiled-coil regions

total: 0 residues

--------------------------
Final Results (k = 9/23):

65.2%: nuclear
21.7%: mitochondrial
13.0%: cytoplasmic

>> prediction for CG58504-01 is nuc (k = 23)

A search of the NOV68a protein against the Geneseq database, a proprietary database that contains sequences published in patents and patent publication, yielded several homologous proteins shown in Table 68D.

TABLE 68D
Geneseq Results for NOV68a

| | | NOV68a | Identities/ | |
|---|---|---|---|---|
| | | Residues/ | Similarities for | Ex- |
| Geneseq | Protein/Organism/Length | Match | the Matched | pect |
| Identifier | [Patent #, Date] | Residues | Region | Value |
| AAB74944 | Human ADAM type metal | 24 . . . 1574 | 733/1718 (42%) | 0.0 |
| | protease MDTS1 protein SEQ ID | 21 . . . 1673 | 941/1718 (54%) | |
| | NO:1 - Homo sapiens, 1686 aa. | | | |
| | [JP2001008687-A, 16-Jan.-2001] | | | |
| AAE00913 | Human 27875 ADAM-TS | 24 . . . 1574 | 731/1718 (42%) | 0.0 |
| | protein, alternative version - | 21 . . . 1673 | 939/1718 (54%) | |
| | Homo sapiens, 1686 aa. | | | |
| | [WO200131034-A1, 03-May- | | | |
| | 2001] | | | |
| AAE00934 | Human 27875 ADAM-TS | 24 . . . 1574 | 731/1718 (42%) | 0.0 |
| | disintegrin and metalloproteinase) - | 21 . . . 1673 | 939/1718 (54%) | |
| | Homo sapiens, 1686 aa. | | | |
| | [WO200131034-A1, 03-May- | | | |
| | 2001] | | | |
| AAB86949 | Human metalloprotease MPTS- | 24 . . . 1574 | 731/1718 (42%) | 0.0 |
| | 19 protein - Homo sapiens, 1690 | 25 . . . 1677 | 938/1718 (54%) | |
| | aa. [DE10107360-A1, 06-Sep.- | | | |
| | 2001] | | | |
| AAB72283 | Human ADAMTS-7 amino acid | 24 . . . 903 | 483/935 (51%) | 0.0 |
| | sequence - Homo sapiens, 997 aa. | 21 . . . 936 | 609/935 (64%) | |
| | [WO200111074-A2, 15-Feb.- | | | |
| | 2001] | | | |

In a BLAST search of public sequence databases, the NOV68a protein was found to have homology to the proteins shown in the BLASTP data in Table 68E.

TABLE 68E
Public BLASTP Results for NOV68a

| | | NOV68a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P58397 | ADAMTS-12 precursor (EC | 1 . . . 1593 | 1593/1593 (100%) | 0.0 |
| | 3.4.24.-) (A disintegrin and | 1 . . . 1593 | 1593/1593 (100%) | |
| | metalloproteinase with | | | |
| | thrombospondin motifs 12) | | | |
| | (ADAM-TS 12) (ADAM-TS 12) - | | | |
| | Homo sapiens (Human), 1593 | | | |
| | aa. | | | |
| CAD60967 | Metalloprotease disintegrin 12 | 1 . . . 1588 | 1283/1600 (80%) | 0.0 |
| | protein - Mus musculus | 1 . . . 1595 | 1399/1600 (87%) | |
| | (Mouse), 1600 aa. | | | |
| Q8BKYI | ADAMTS-12 precursor - Mus | 1 . . . 1004 | 889/1009 (88%) | 0.0 |
| | musculus (Mouse), 1009 aa | 1 . . . 1009 | 939/1009 (92%) | |
| | (fragment). | | | |
| CAC38921 | Sequence 2 from Patent | 24 . . . 1574 | 731/1718 (42%) | 0.0 |
| | W0013 1034 - Homo sapiens | 21 . . . 1673 | 939/1718 (54%) | |
| | (Human), 1686 aa. | | | |
| Q9UKP4 | ADAMTS-7 precursor (EC | 24 . . . 903 | 485/935 (51%) | 0.0 |
| | 3.4.24.-) (A disintegrin and | 21 . . . 936 | 611/935 (64%) | |
| | metalloproteinase with | | | |
| | thrombospondin motifs 7) | | | |
| | (ADAM-TS 7) (ADAM-TS7) - | | | |
| | Homo sapiens (Human), 997 aa. | | | |

PFam analysis indicates that the NOV68a protein contains the domains shown in the Table 68F.

TABLE 68F
Domain Analysis of NOV68a

| | | Identities/ | |
|---|---|---|---|
| | | Similarities | |
| Pfam Domain | NOV68a Match Region | for the Matched Region | Expect Value |
| Pep_M12B_propep | 105 . . . 222 | 27/128(21%) | 9.2e-05 |
| | | 80/128 (62%) | |
| Reprolysin | 246 . . . 456 | 66/224 (29%) | 1.3e-15 |
| | | 149/224 (67%) | |
| tsp_1 | 546 . . . 596 | 23/53 (43%) | 2.2e-13 |
| | | 38/53 (72%) | |
| tsp_1 | 827 . . . 881 | 15/64 (23%) | 0.054 |
| | | 39/64 (61%) | |
| tsp_1 | 945 . . . 995 | 17/58(29%) | 7.1e-05 |
| | | 39/58 (67%) | |
| tsp_1 | 1314 . . . 1364 | 13/56 (23%) | 0.036 |
| | | 29/56 (52%) | |
| tsp_1 | 1426 . . . 1471 | 14/54 (26%) | 0.055 |
| | | 32/54 (59%) | |
| tsp_1 | 1474 . . . 1530 | 14/63 (22%) | 0.0045 |
| | | 40/63 (63%) | |

### Example 69

The NOV69 clone was analyzed, and the nucleotide and encoded polypeptide sequences are shown in Table 69A.

TABLE 69A
NOV69 Sequence Analysis

| | | |
|---|---|---|
| NOV69a, CG58510-01 | SEQ ID NO: 1031 | 933 bp |

| | | |
|---|---|---|
| DNA Sequence | ORF Start: ATG at 8 | ORF Stop: at 932 |

GGATTAAATGGAAAACGATCACAATCATAATCATACACATGGCGCGAACAAAAAGACTTTATTAATTA
GTTTTATTATTATTACAAGTTACATGATTGTAGAAGGGTTAGGTGGTTTCTTTACTAACAGTCTTGCG
TTAATTTCAGATGCTGGTCATATGTTAAGTGATTCTATTTCTTTAGGTATTGCTTTAATTGCATTTAC
TTTAGGAGCGAAGCAAGCTAATACAAATAAAACTTTTGGCTACAAAAGGTTTGAAATACTAGCAGCTG
TACTTAATGGTATTACTTTGATGTTAATAGCTATCTATATTTTTTATGAAGCTATTGAGAGATTTAAA
AACCCCCCTGAGGTAGCTTCTACAGGCATGTTAATTATCGCCTTGGTAGGCTTGTTTATTAATATTAT
TGTGGCTTGGATAATGCTGCGCGGGAGCGATGTAGAAGAAAACTTAAATATGCGTGGAGCATATTTGC
ATGTAATAAGCGACATGCTTGGATCTATAGGTGCAGTTATAGCAGCCCTTCTCATTATATTTTTTAGA
TGGGGGTGGGCGGATCCTTTAGCAAGTGTGATTGTAGCAATTTTAGTACTACGTAGCGGCTTTTATGT
AACAAAATCAAGTCTTCATGTATTAATGGAGGGAGCACCAAGCAATATAAATACAAAAGACATTATTA
AAACTATTAAAAAATTCAAAGAAGTTAAAAATATTCATGACTTTCATGTTTGGTCAGTAACTAGCGGA
TTAAACGCGTTATCTTGTCATATTGTTGTAGAAGATACAATGACCATTACTGAAAATGAGTTTTTACT
TAAACGTATAGAACATGAATTCAATCATCAAAATATTCAACATGTCACAATACAGACTGAAACTTCTA
ACAATAATCATAGTGAAAAATTGTTTTGTATCGTGAAAGAAGAAGACAG

NOV69a, CG58510-01

| | | | |
|---|---|---|---|
| Protein Sequence | SEQ ID NO: 1032 | 308 aa | MW at 34326.6kD |

MENDHNHNHTHGANKKTLLISFIIITSYMIVEGLGGFFTNSLALISDAGHMLSDSISLGIALIAFTLG
AKQANTNKTFGYKRFEILAAVLNGITLMLIAIYIFYEAIERFKNPPEVASTGMLIIALVGLFINIIVA
WIMLRGSDVEENLNMRGAYLHVISDMLGSIGAVIAALLIIFFRWGWADPLASVIVAILVLRSGFYVTK
SSLHVLMEGAPSNINTKDIIKTIKKFKEVKNIHDFHVWSVTSGLNALSCHIVVEDTMTITENEFLLKR
IEHEFNHQNIQHVTIQTETSNNNHSEKLFCIVKEED

Further analysis of the NOV69a protein yielded the following properties shown in Table 69B.

TABLE 69B
Protein Sequence Properties NOV69a

| | |
|---|---|
| SignalP analysis: | Cleavage site between residues 34 and 35 |
| PSORT II analysis: | |

| PSG: | a new signal peptide prediction method | |
|---|---|---|
| | N-region: length 4; pos.chg 0; neg.chg 2 | |
| | H-region: length 10; peak value 0.00 | |
| | PSG score: −4.40 | |
| GvH: | von Heijne's method for signal seq. recognition | |
| | GvH score (threshold: −2.1): −6.24 | |
| | possible cleavage site: between 43 and 44 | |
| | >>> Seems to have no N-terminal signal peptide | |
| ALOM: | Klein et al's method for TM region allocation | |
| | Init position for calculation: 1 | |
| | Tentative number of TMS(s) for the threshold 0.5: 6 | |
| | INTEGRAL Likelihood = −4.46 | Transmembrane 18-34 |
| | INTEGRAL Likelihood = −1.65 | Transmembrane 51-67 |
| | INTEGRAL Likelihood = −6.32 | Transmembrane 85-101 |
| | INTEGRAL Likelihood = −11.41 | Transmembrane 122-138 |
| | INTEGRAL Likelihood = −9.55 | Transmembrane 162-178 |
| | INTEGRAL Likelihood = −4.73 | Transmembrane 186-202 |

| | PERIPHERAL Likelihood = 2.54 (at 241) |
|---|---|
| | ALOM score: −11.41 (number of TMSs: 6) |
| MTOP: | Prediction of membrane topology (Hartmann et al.) |
| | Center position for calculation: 25 |
| | Charge difference: −3.0 C(−1.0) - N( 2.0) |
| | N >= C: N-terminal side will be inside |

>>> membrane topology: type 3a
MITDISC: discrimination of mitochondrial targeting seq

| | | | |
|---|---|---|---|
| R content: | 0 | Hyd Moment (75): | 6.56 |
| Hyd Moment (95): | 3.47 | G content: | 0 |
| D/E content: | 2 | S/T content: | 0 |
| Score: −7.29 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 7.5%
NLS Score: −0.47

KDEL: ER retention motif in the C-terminus: none
ER Membrane Retention Signals:

KKXX-like motif in the C-terminus: VKEE

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination

Prediction.: cytoplasmic
Reliability: 94.1

COIL: Lupas's algorithm to detect coiled-coil regions

total: 0 residues

--------------------------
Final Results (k = 9/23):

66.7%: endoplasmic reticulum
22.2%: mitochondrial
11.1%: vesicles of secretory system

>> prediction for CG58510-01 is end (k = 9)

A search of the NOV69a protein against the Geneseq database, a proprietary database that contains sequences published in patents and patent publication, yielded several homologous proteins shown in Table 69C.

TABLE 69C
Geneseq Results for NOV69a

| | | NOV69a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB48454 | Listeria monocytogenes protein | 5 . . . 296 | 163/294 (55%) | 2e-91 |
| | #1158 - Listeria monocytogenes, | 11 . . . 299 | 225/294 (76%) | |
| | 303 aa. [WO200177335-A2, 18- | | | |
| | Oct.-2001] | | | |
| ABP39345 | Staphylococcus epidermidis ORF | 3 . . . 302 | 157/300 (52%) | 3e-91 |
| | amino acid sequence SEQ ID | 35 . . . 334 | 220/300 (73%) | |
| | NO:4190 - Staphylococcus | | | |
| | epidermidis, 342 aa. [US6380370- | | | |
| | B1, 30-Apr.-2002] | | | |
| ABP40773 | Staphylococcus epidermidis ORF | 1 . . . 299 | 112/305 (36%) | 4e-56 |
| | amino acid sequence SEQ ID | 47 . . . 351 | 188/305 (60%) | |
| | NO:5618 - Staphylococcus | | | |
| | epidermidis, 359 aa. [US6380370- | | | |
| | B1, 30-Apr.-2002] | | | |
| AAU61849 | Propionibacterium acnes | 6 . . . 298 | 108/299 (36%) | 8e-52 |
| | immunogenic protein #22745 - | 10 . . . 306 | 185/299 (61%) | |
| | Propionibacterium acnes, 310 aa. | | | |
| | [WO200181581-A2, 01-Nov.- | | | |
| | 2001] | | | |
| AAB76797 | Corynebacterium glutamicum | 3 . . . 300 | 107/302 (35%) | 2e-51 |
| | MCT protein SEQ ID NO:576 - | 18 . . . 317 | 180/302 (59%) | |
| | Corynebacterium glutamicum, 318 | | | |
| | aa. [WO200100805-A2, 04-Jan.- | | | |
| | 2001] | | | |

In a BLAST search of public sequence databases, the NOV69a protein was found to have homology to the proteins shown in the BLASTP data in Table 69D.

TABLE 69D
Public BLASTP Results for NOV69a

| | | NOV69a | Identities/ | |
|---|---|---|---|---|
| Protein | | Residues/ | Similarities for | Ex- |
| Accession | | Match | the Matched | pect |
| Number | Protein/Organism/Length | Residues | Portion | Value |
| O07084 | Cation transport protein YRDO - | 7 . . . 307 | 206/301 (68%) | e-122 |
| | Bacillus subtilis, 311 aa. | 3 . . . 303 | 253/301 (83%) | |
| P71023 | CzcD - Bacillus subtilis, 295 aa | 21 . . . 307 | 194/287 (67%) | e-115 |
| | (fragment). | 1 . . . 287 | 242/287 (83%) | |
| Q8NVF2 | CzrB protein - Staphylococcus | 3 . . . 302 | 161/300 (53%) | 2e-95 |
| | aureus (strain MW2), 326 aa. | 8 . . . 307 | 225/300 (74%) | |
| Q99SB4 | CzrB protein (Cation-efflux | 3 . . . 302 | 161/300 (53%) | 2e-95 |
| | system membrane protein | 7 . . . 306 | 225/300 (74%) | |
| | homolog) - Staphylococcus aureus | | | |
| | (strain Mu50 / ATCC 700699), | | | |
| | and, 325 aa. | | | |
| Q9ZNF5 | CzrB protein - Staphylococcus | 3 . . . 302 | 161/300 (53%) | 2e-95 |
| | aureus, 325 aa. | 7 . . . 306 | 225/300 (74%) | |

PFam analysis indicates that the NOV69a protein contains the domains shown in the Table 69E.

TABLE 69E
Domain Analysis of NOV69a

| | | Identities/ | |
|---|---|---|---|
| | NOV69a | Similarities | |
| | Match | for the | |
| Pfam Domain | Region | Matched Region | Expect Value |
| Cation_efflux | 18 . . . 296 | 102/303 (34%) | 1.5e-106 |
| | | 245/303 (81%) | |

### Example 70

The NOV70 clone was analyzed, and the nucleotide and encoded polypeptide sequences are shown in Table 70A.

TABLE 70A
NOV70 Sequence Analysis

| | | |
|---|---|---|
| NOV70a, CG59309-01 | SEQ ID NO: 1033 | 1375 bp |

| | | |
|---|---|---|
| DNA Sequence | ORF Start: ATG at 96 | ORF Stop: TAA at 1362 |

GGGACGCCGGACGCCGTCCGGACATTCGGCGCGCTTGCCACGATCTTGGACGGGTCTCGGGCCTCGAC
CTTTGAATTCCCCGCTCCGGCTCCAAGATGTCAGCAACGCTGATCCTGGAGCCCCCAGGCCGCTGCTG
CTGGAACGAGCCGGTGCGCATTGCCGTGCGCGGCCTGGCCCCGGAGCAGCGGGTTACGCTGCGCGCGT
CCCTGCGCGACGAGAAGGGCGCGCTCTTCCGGGCCCACGCGCGCTACTGCGCCGACGCCCGCGGCGAG
CTGGACCTGGAGCGCGCACCCGCGCTGGGCGGCAGCTTCGCGGGACTCGAGCCCATGGGGCTGCTCTG
GGCCCTGGAACCCGAGAAGCCTTTTTGGCGCTTCCTGAAGCGGGACGTACAGATTCCTTTTGTCGTGG
AGTTGGAGGTGCTGGACGGCCACGACCCCGAGCCTGGACGGCTGCTGTGCCAGGCGCAGCACGAGCGC
CACTTCCTCCCGCCAGGGGTGCGGCGCCAGTCGGTGCGAGCGGGCCGGGTGCGCGCCACGCTCTTCCT
GCCGCCAGGTGAGCCTGGACCCTTCCCAGGGATCATTGACATCTTTGGTATTGGAGGGGGCCTCTTGG
AATATCGAGCCAGCCTCCTTGCTGGCCATGGCTTTGCCACGTTGGCTCTAGCTTATTATAACTTTGAA
GATCTCCCCAATAACATGGACAACATATCCCTGGAGTACTTCGAAGAAGCCGTATGCTACATGCTTCA
ACATCCCCAGGTTAAAGGCCCAGGCATTGGGCTTTTGGGCATTTCTCTAGGAGCTGATATTTGTCTCT
CAATGGCCTCATTCTTGAAGAATGTCTCAGCCACAGTTTCCATCAATGGATCTGGGATCAGTGGGAAC
ACAGCCATCAACTATAAGCACAGTAGCATTCCACCATTGGGCTATGACCTGAGGAGAATCAAGGTAGC
TTTCTCAGGCCTCGTGGACATTGTGGATATAAGGAATGCTCTCGTAGGAGGGTACAAGAACCCCAGCA
TGATTCCAATAGAGAAGGCCCAGGGGCCCATCCTGCTCATTGTTGGTCAGGATGACCATAACTGGAGA
AGTGAGTTGTATGCCCAAACAGTCTCTGAACGGTTACAGGCCCATGGAAAGGAAAAACCCCAGATCAT
CTGTTACCCTGGGACTGGGCATTACATCGAGCCTCCTTACTTCCCCCTGTGCCCAGCTTCCCTTCACA
GATTACTGAACAAACATGTTATATGGGGTGGGGAGCCCAGGGCTCATTCTAAGGCCCAGGAAGATGCC
TGGAAGCAAATTCTAGCCTTCTTCTGCAAACACCTGGGAGGTACCCAGAAAACAGCTGTCCCTAAATT
GTAATGCATTTGTCT

NOV70a, CG59309-01

| | | | |
|---|---|---|---|
| Protein Sequence | SEQ ID NO: 1034 | 422 aa | MW at 46455.1kD |

MSATLILEPPGRCCWNEPVRIAVRGLAPEQRVTLRASLRDEKGALFRAHARYCADARGELDLERAPAL
GGSFAGLEPMGLLWALEPEKPFWRFLKRDVQIPFVVELEVLDGHDPEPGRLLCQAQHERHFLPPGVRR
QSVRAGRVRATLFLPPGEPGPFPGIIDIFGIGGGLLEYRASLLAGHGFATLALAYYNFEDLPNNMDNI
SLEYFEEAVCYMLQHPQVKGPGIGLLGISLGADICLSMASFLKNVSATVSINGSGISGNTAINYKHSS
IPPLGYDLRRIKVAFSGLVDIVDIRNALVGGYKNPSMIPIEKAQGPILLIVGQDDHNWRSELYAQTVS
ERLQAHGKEKPQIICYPGTGHYIEPPYFPLCPASLHRLLNKHVIWGGEPRAHSKAOEDAWKQILAFFC
KHLGGTQKTAVPKL

| | | |
|---|---|---|
| NOV70b, 278901386 | SEQ ID NO: 1035 | 1285 bp |

| | | |
|---|---|---|
| DNA Sequence | ORF Start: at 2 | ORF Stop: end of sequence |

CACCGGATCCACCATGTCAGCAACGCTGATCCTGGAGCCCCCAGGCCGCTGCTGCTGGAACGAGCCGG
TGCGCATTGCCGTGCGCGGCCTGGCCCCGGAGCAGCGGGTTACGCTGCGCGCGTCCCTGCGCGACGAG
AAGGGCGCGCTCTTCCGGGCCCACGCGCGCTACTGCGCCGACGCCTGCGGCGAGCTGGACCTGGAGCG
CGCACCCGCGCTGGGCGGCAGCTTCGCGGGACTCGAGCCCATGGGGCTGCTCTGGGCCCTGGAACCCG
AGAAGCCTTTTTGGCGCTTCCTGAAGCGGGACGTACAGATTCCTTTTGTCGTGGAGTTGGAGGTGCTG
GACGGCCACGACCCCGAGCCTGGACGGCTGCTGTGCCAGGCGCAGCACGAGCGCCACTTCCTCCCGCC
AGGGGTGCGGCGCCAGTCGGTGCGAGCGGGCCGGGTGCGCGCCACGCTCTTCCTGCCGCCAGGACCTG
GACCCTTCCCAGGGATCATTGACATCTTTGGTATTGGAGGGGGCCTCTTGGAATATCGAGCCAGCCTC
CTTGCTGGCCATGGCTTTGCCACGTTGGCTCTAGCTTATTATAACTTTGAAGATCTCCCCAATAACAT
GGACAACATATCCCTGGAGTACTTCGAAGAAGCCGTATGCTACATGCTTCAACATCCCCAGGTAAAAG
GCCCAGGCATTGGGCTTTTGGGCATTTCTCTAGGAGCTGATATTTGTCTCTCAATGGCCTCATTCTTG
AAGAATGTCTCAGCCACAGTTTCCATCAATGGATCTGGGATCAGTGGGAACACAGCCATCAACTATAA
GCACAGTAGCATTCCACCATTGGGCTATGACCTGAGGAGAATCAAGGTAGCTTTCTCAGGCCTCGTGG
ACATCGTGGATATAAGGAATGCTCTCGTAGGAGGGTACAAGAACCCCAGCATGATTCCAATAGAGAAG
GCCCAGGGGCCCATCCTGCTCATTGTTGGTCAGGATGACCATAACTGGAGAAGTGAGTTGTATGCCCA
AACAGTCTCTGAACGGTTACAGGCCCATGGAAAGGAAAAACCCCAGATCATCTGTTACCCTGGGACTG
GGCATTACATCGAGCCTCCTTACTTCCCCCTGTGCCCAGCTTCCCTTCACAGATTACTGAACAAACAT
GTTATATGGGGTGGGGAGCCCAGGGCTCATTCTAAGGCCCAGGAAGATGCCTGGAAGCAAATTCTAGC
CTTCTTCTGCAAACACCTGGGAGGTACCCAGAAAACAGCTGTCCCTAAATTGGTCGACGGC

NOV70b, 278901386

| | | | |
|---|---|---|---|
| Protein Sequence | SEQ ID NO: 1036 | 428 aa | MW at 46890.5kD |

TGSTMSATLILEPPGRCCWNEPVRIAVRGLAPEQRVTLRASLRDEKGALFRAHARYCADACGELDLER
APALGGSFAGLEPMGLLWALEPEKPFWRFLKRDVQIPFVVELEVLDGHDPEPGRLLCQAQHERHFLPP
GVRRQSVRAGRVRATLFLPPGPGPFPGIIDIFGIGGGLLEYRASLLAGHGFATLALAYYNFEDLPNNM
DNISLEYFEEAVCYMLQHPQVKGPGIGLLGISLGADICLSMASFLKNVSATVSINGSGISGNTAINYK
HSSIPPLGYDLRRIKVAFSGLVDIVDIRNALVGGYKNPSMIPIEKAQGPILLIVGQDDHNWRSELYAQ
TVSERLQAHGKEKPQIICYPGTGHYIEPPYFPLCPASLHRLLNKNVIWGGEPRAHSKAQEDAWKQILA
FFCKHLGGTQKTAVPKLVDG

A ClustalW comparison of the above protein sequences yields the following sequence alignment shown in Table 70B.

TABLE 70B
Comparison of the NOV70 protein sequences.

| | |
|---|---|
| NOV70a | ----MSATLILEPPGRCCWNEPVRIAVRGLAPEQRVTLRASLRDEKGALFRAHARYCADA |
| NOV70b | TGSTMSATLILEPPGRCCWNEPVRIAVRGLAPEQRVTLRASLRDEKGALFRAHARYCADA |
| NOV7ca | RGELDLERAPALGGSFAGLEPMGLLWALEPEKPFWRFLKRDVQIPFVVELEVLDGHDPEP |
| NOV70b | CGELDLERAPALGGSFAGLEPMGLLWALEPEKPFWRFLKRDVQIPFVVELEVLDGHDPEP |
| NOV70a | GRLLCQAQHERHFLPPGVRRQSVRAGRVRATLFLPPGEPGPFPGIIDIFGIGGGLLEYRA |
| NOV70b | GRLLCQAQHERHFLPPGVRRQSVRAGRVRATLFLPPG-PGPFPGIIDIFGIGGGLLEYRA |
| NOV70a | SLLAGHGFATLALAYYNFEDLPNNNDNISLEYFEEAVCYMLQHPQVKGPGIGLLGISLGA |
| NOV70b | SLLAGHGFATLALAYYNFEDLPNNMDNISLEYFEEAVCYMLQHPQVKGPGIGLLGISLGA |
| NOV70a | DICLSMASFLKNVSATVSINGSGISGNTAINYKHSSIPPLGYDLRRIKVAFSGLVDIVDI |
| NOV70b | DICLSMASFLKNVSATVSINGSGISGNTAINYKHSSIPPLGYDLRRIKVAFSGLVDIVDI |
| NOV70a | RNALVGGYKNPSMIPIEKAQGPILLIVGQDDHNWRSELYAQTVSERLQAHGKEKPQIICY |
| NOV70b | RNALVGGYKNPSMIPIEKAQGPILLIVGQDDHNWRSELYAQTVSERLQAHGKEKPQIICY |
| NOV70a | PGTGHYIEPPYFPLCPASLHRLLNKHVIWGGEPRAHSKAQEDAWKQILAFFCKHLGGTQK |
| NOV70b | PGTGHYIEPPYFPLCPASLHRLLNKHVIWGGEPRAHSKAQEDAWKQILAFFCKHLGGTQK |
| NOV70a | TAVPKL--- |
| NOV70b | TAVPKLVDG |
| NOV70a | (SEQ ID NO: 1034) |
| NOV70b | (SEQ ID NO: 1036) |

Further analysis of the NOV70a protein yielded the following properties shown in Table 70C.

TABLE 70C
Protein Sequence Properties NOV7Oa

| | |
|---|---|
| SignalP analysis: | No Known Signal Sequence Indicated |
| PSORT II analysis: | |

| PSG: | a new signal peptide prediction method |
|---|---|
| | N-region: length 8; pos..chg 0; neg.chg 1 |
| | H-region: length 3; peak value 0.00 |
| | PSG score: −4.40 |
| GvH: | von Heijne's method for signal seq. recognition |
| | GvH score (threshold: −2.1): −7.81 |
| | possible cleavage site: between 13 and 14 |

>>> Seems to have no N-terminal signal peptide

| ALOM: | Klein et al≡s method for TM region allocation |
|---|---|
| | Init position for calculation: 1 |
| | Tentative number of TMS(s) for the threshold 0.5: 1 |
| | Number of TMS(s) for threshold 0.5: 0 |
| | PERIPHERAL Likelihood = 1.75 (at 285) |
| | ALOM score: −1.86 (number of TMSs: 0) |

MITDISC: discrimination of mitochondrial targeting seq

| | | | |
|---|---|---|---|
| R content: | 1 | Hyd Moment (75): | 7.13 |
| Hyd Moment (95): | 4.74 | G content: | 1 |
| D/E content: | 2 | S/T content: | 2 |
| Score: −6.03 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 10.4%
NLS Score: −0.47

KDEL: ER retention motif in the C-terminus: none
ER Membrane Retention Signals:

KXXX-like motif in the C-terminus: AVPK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

| | type 1: none |
|---|---|
| | type 2: none |
| NMYR: | N-myristoylation pattern : none |

Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| NNCN: | Reinhardt's method for Cytoplasmic/Nuclear discrimination |
|---|---|
| | prediction: cytoplasmic |
| | Reliability: 94.1 |
| COIL: | Lupas's algorithm to detect coiled-coil regions |
| | total: 0 residues |

--------------------------
Final Results (k = 9/23):

```
65.2%: cytoplasmic
13.0%: nuclear
  8.7%: mitochondrial
  8.7%: peroxisomal
  4.3%: plasma membrane
```

>> prediction for CG59309-01 is cyt (k = 23)

A search of the NOV70a protein against the Geneseq database, a proprietary database that contains sequences published in patents and patent publication, yielded several homologous proteins shown in Table 70D.

TABLE 70D
Geneseq Results for NOV70a

| | | NOV70a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE25382 | Human NZMS-6 protein - Homo | 1 . . . 422 | 421/422 (99%) | 0.0 |
| | sapiens, 421 aa. [WO200246385- | 1 . . . 421 | 421/422 (99%) | |
| | A2, 13-Jun.-2002] | | | |
| AAU76350 | Human Acyl-CoA thioesterase | 1 . . . 422 | 420/422 (99%) | 0.0 |
| | 56939 - Homo sapiens, 421 aa. | 1 . . . 421 | 420/422 (99%) | |
| | [WO200208274-A2, 31-Jan.- | | | |
| | 2002] | | | |
| AAM41490 | Human polypeptide SEQ ID NO | 1 . . . 422 | 296/422 (70%) | e-178 |
| | 6421 - Homo sapiens, 494 aa. | 74 . . . 494 | 341/422 (80%) | |
| | [WO2001533 12-A1, 26-Jul.- | | | |
| | 2001] | | | |
| AAM39704 | Human polypeptide SEQ ID NO | 1 . . . 422 | 296/422 (70%) | e-178 |
| | 2849 - Homo sapiens, 483 aa. | 63 . . . 483 | 341/422 (80%) | |
| | [WO2001 53312-A1, 26-Jul.- | | | |
| | 2001] | | | |
| AAY71112 | Human Hydrolase protein- 10 | 1 . . . 422 | 296/422 (70%) | e- 178 |
| | (HYDRL-10) - Homo sapiens, | 63 . . . 483 | 341/422 (80%) | |
| | 483 aa. [WO200028045-A2, 18- | | | |
| | May-2000] | | | |

In a BLAST search of public sequence databases, the NOV70a protein was found to have homology to the proteins shown in the BLASTP data in Table 70E.

TABLE 70E
Public BLASTP Results for NOV70a

| | | NOV70a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD35497 | Sequence 1 from Patent | 1 . . . 422 | 420/422 (99%) | 0.0 |
| | WO0208274 - Homo sapiens | 1 . . . 421 | 420/422 (99%) | |
| | (Human), 421 aa. | | | |
| Q8N9L9 | Hypothetical protein FLJ36904 - | 1 . . . 422 | 417/422 (98%) | 0.0 |
| | Homo sapiens (Human), 421 aa. | 1 . . . 421 | 417/422 (98%) | |
| CAD62354 | Human full-length cDNA clone | 70 . . . 422 | 352/353 (99%) | 0.0 |
| | CSODIO29YHO6 of Placenta of | 1 . . . 352 | 352/353 (99%) | |
| | Homo sapiens (human) - Homo | | | |
| | sapiens (Human), 352 aa | | | |
| | (fragment). | | | |
| Q8BWN8 | Peroxisomal long chain acyl-CoA | 1 . . . 422 | 313/422 (74%) | 0.0 |
| | thioesterase IB - Mus musculus | 1 . . . 421 | 363/422 (85%) | |
| | (Mouse), 421 aa. | | | |
| Q8BL20 | Peroxisomal long chain acyl-CoA | 1 . . . 422 | 313/422 (74%) | 0.0 |
| | thioesterase IB - Mus musculus | 1 . . . 421 | 362/422 (85%) | |
| | (Mouse), 421 aa. | | | |

PFam analysis indicates that the NOV70a protein contains the domains shown in the Table 70F.

TABLE 70F
Domain Analysis of NOV70a

| | NOV70a | Identities/Similarities | Expect |
|---|---|---|---|
| Pfam Domain | Match Region | for the Matched Region | Value |
| Bile_Hydr_Trans | 3 . . . 152 | 84/157 (54%) | 1.9e−82 |
| | | 140/157 (89%) | |
| DLH | 144 . . . 411 | 65/309 (21%) | 0.33 |
| | | 170/309 (55%) | |

### Example 71

The NOV71 clone was analyzed, and the nucleotide and encoded polypeptide sequences are shown in Table 71A.

TABLE 71A
NOV71 Sequence Analysis

| | | |
|---|---|---|
| NOV71a, CG59490-01 | SEQ ID NO: 1037 | 660 bp |

| | | |
|---|---|---|
| DNA Sequence | ORF Start: at 1 | ORF Stop: end of sequence |

TCACTGGGGGCTGCGACGTCTCGGCCAGGAGGCACCCCTGGCAGGGAGGAGTTGGAGGCTTGCGCGTT
TAGAGTGCAGGTGGGGCAGCTGAGGCTCTATGAGGACGACCAGCGGACGAAGGTGGTTGAGATCGTCC
GTCACCCCCAGTACAACGAGAGCCTGTCTGCCCAGGGCGGTGCGGACATCGCCCTGCTGAAGCTGGAG
GCCCCGGTGCCGCTGTCTGAGCTCATCCACCCGGTCTCGCTCCCGTCTGCCTCCCGGGACGTGCCCTC
GGGGAAGACCTGCTGGGTGACCGGCTGGGGTGTCATTGGACGTGGAGAACTACTGCCCTGGCCCCTCA
GCTTGTGGGAGGCGACGGTGAAGGTCAGGAGCAACGTCCTCTGTAACCAGACCTGTCGCCGCCGCTTT
CCTTCCAACCACACTGAGCGGTTTGAGCGGCTCATCAAGGACGACATGCTGTGTGCCGGGGACGGGAA
CCACGGCTCCTGGCCAGGCGACAACGGGGGCCCCCTCCTGTGCAGGCGGAATTGCACCTGGGTCCAGG
TGGAGGTGGTGAGCTGGGGCAAACTCTGCGGCCTTCGCGGCTATCCCGGCATGTACACCCGCGTGACG
AGCTACGTGTCCTGGATCCGCCAGTACGTCCCGCCGTTCCCCAGACGC

NOV71a, CG59490-01

| | | | |
|---|---|---|---|
| Protein Sequence | SEQ ID NO: 1038 | 220 aa | MW at 24527.8kD |

SLGAATSRPGGTPGREELEACAFRVQVGQLRLYEDDQRTKVVEIVRHPQYNESLSAQGGADIALLKLE
APVPLSELIHPVSLPSASRDVPSGKTCWVTGWGVIGRGELLPWPLSLWEATVKVRSNVLCNQTCRRRF
PSNHTERFERLIKDDMLCAGDGNHGSWPGDNGGPLLCRRNOTWVQVEVVSWGKLCGLRGYPGMYTRVT
SYVSWIRQYVPPFPRR

| | | |
|---|---|---|
| NOV71b, 207639512 | SEQ ID NO: 1039 | 672 bp |

| | | |
|---|---|---|
| DNA Sequence | ORF Start: at 1 | ORF Stop: end of sequence |

AGATCTTCACTGGGGGCTGCGACGTCTCGGCCAGGAGGCACCCCTGGCAGGGAGGAGTTGGAGGCTTG
CGCGTTTAGAGTGCAGGTGGGGCAGCTGAGGCTCTATGAGGACGACCAGCGGACGAAGGTGGTTGAGA
TCGTCCGTCACCCCCAGTACAACGAGAGCCTGTCTGCCCAGGGCGGTGCGGACATCGCCCTGCTGAAG
CTGGAGGCCCCGGTGCCGCTGTCTGAGCTCATCCACCCGGTCTCGCTCCCGTCTGCCTCCCTGGACGT
GCCCTCGGGGAAGACCTGCTGGGTGACCGGCTGGGGTGTCATTGGACGTGGAGAACTACTGCCCTGGC
CCCTCAGCTTGTGGGAGGCGACGGTGAAGGTCAGGAGCAGCGTCCTCTGTAACCAGACCTGTCGCCGC
CGCTTTCCTTCCAACCACACTGAGCGGTTTGAGCGGCTCATCAAGGACGACATGCTGTGTGCCGGGGA
CGGGAACCACGGCTCCTGGCCAGGCGACAACGGGGGCCCCCTCCTGTGCAGGCGGAATTGCACCTGGG
TCCAGGTGGAGGTGGTGAGCTGGGGCAAACTCTGCGGCCTTCGCGGCTATCCCGGCATGTACACCCGC
GTGACGAGCTACGTGTCCTGGATCCGCCAGTACGTCCCGCCGTTCCCCAGACGCCTCGAG

NOV71b, 207639512

| | | | |
|---|---|---|---|
| Protein Sequence | SEQ ID NO: 1040 | 224 aa | MW at 24943.3kD |

RSSLGAATSRPGGTPGREELEACAFRVQVGQLRLYEDDQRTKVVEIVRHPQYNESLSAQGGADIALLK
LEAPVPLSELIHPVSLPSASLDVPSGKTCWVTGWGVIGRGELLPWPLSLWEATVKVRSSVLCNQTCRR
RFPSNHTERFERLIKDDMLCAGDGNHGSWPGDNGGPLLCRRNCTWVQVEVVSWGKLCGLRGYPGMYTR
VTSYVSWIRQYVPPFPRRLE

| | | |
|---|---|---|
| NOV71c, 207639476 | SEQ ID NO: 1041 | 672 bp |

| | | |
|---|---|---|
| DNA Sequence | ORF Start: at 1 | ORF Stop: end of sequence |

AGATCTTCACTGGGGGCTGCGACGTCTCGGCCAGGAGGCACCCCTGGCAGGGAGGAGTTGGAGGCTTG
CGCGTTTAGAGTGCAGGTGGGGCAGCTGAGGCTCTATGAGGACGACCAGCGGACGAAGGTGGTTGAGA
TCGTCCGTCACCCCCAGTACAACGAGAGCCTGTCTGCCCAGGGCGGTGCGGACATCGCCCTGCTGAAG
CTGGAGGCCCCGGTGCCGCTGTCTGAGCTCATCCACCCGGTCTCGCTCCCGTCTGCCTCCCTGGACGT
GCCCTCGGGGAAGACCTGCTGGGTGACCGGCTGGGGTGTCATTGGACGTGGAGAACTACTGCCCTGGC
CCCTCAGCTTGTGGGAGGCGACGGTGAAGGTCAGGAGCAACGTCCTCTGTAACCAGACCTGTCGCCGC
CGCTTTCCTTCCAACCACACTGAGCGGTTTGAGCGGCTCATCAAGGACGACATGCTGTGTGCCGGGGA
CGGGAACCACGGCTCCTGGCCAGGCGACAACGGGGGCCCCCTCCTGTGCAGGCGGAATTGCACCTGGG
TCCAGGTGGAGGTGGTGAGCTGGGGCAAACTCTGCGGCCTTCGCGGCTATCCCGGCATGTACACCCGC
GTGACGAGCTACGTGTCCTGGATCCGCCAGTACGTCCCGCCGTTCCCCAGACGCCTCGAG

NOV71c, 207639476

| | | | |
|---|---|---|---|
| Protein Sequence | SEQ ID NO: 1042 | 224 aa | MW at 24970.3kD |

RSSLGAATSRPGGTPGREELEACAFRVQVGQLRLYEDDQRTKVVEIVRHPQYNESLSAQGGADIALLK
LEAPVPLSELIHPVSLPSASLDVPSGKTCWVTGWGVIGRGELLPWPLSLWEATVKVRSNVLCNQTCRR
RFPSNHTERFERLIKDDMLCAGDGNHGSWPGDNGGPLLCRRNCTWVQVEVVSWGKLCGLRGYPGMYTR
VTSYVSWIRQYVPPFPRRLE

| | | |
|---|---|---|
| NOV71d, 207639523 | SEQ ID NO: 1043 | 672 bp |

| | | |
|---|---|---|
| DNA Sequence | ORF Start: at 1 | ORF Stop: end of sequence |

AGATCTTCACTGGGGGCTGCGACGTCTCGGCCAGGAGGCACCCCTGGCAGGGAGGAGTTGGAGGCTTG
CGCGTTTAGAGTGCAGGTGGGGCAGCTGAGGCTCTATGAGGACGACCAGCGGACGAAGGTGGTTGAGA
TCGTCCGTCACCCCCAGTACAACGAGAGCCTGTCTGCCCAGGCCGGTGCGGACATCGCCCTGCTGAAG
CTGGAGGCCCCGGTGCCGCTGTTGAGCTCATCCACCCGGTCTCGTCTCCCGTCTGCCTCCCTGGACGT
GCCCTCGGGGAAGACCTGCTGGGTGACCGGCTGGGGTGTCATTGGACGTGGAGAACTACTGCCCTGGC
CCCTCAGCTTGTGGGAGGCGACGGTGAAGGTCAGGAGCAACGTCCTCTGTAACCAGACCTGTCGCCGC
CGCTTTCCTTCCAACCACACTGAGCGGTTTGAGCGGCTCATCAAGGACGACATGCTGTGTGCCGGGGA
CGGGAACCACGGCTCCTGGCCAGGCGACAACGGGGGCCCCCTCCTGTGCAGGCGGAATTGCACCTGGG
TCCAGGTGGAGGTGGTGAGCTGGGGCAAACTCTGCGGCCTTCGCGGCTATCCCGGCATGTACACCCGC
GTGACGAGCTACGTGTCCTGGATCCGCCAGTACGTCCCGCCGTTCCCCAGACGCCTCGAG

NOV71d, 207639523

| | | | |
|---|---|---|---|
| Protein Sequence | SEQ ID NO: 1044 | 224 aa | MW at 24984.3kD |

RSSLGAATSRPGGTPGREELEACAFRVQVGQLRLYEDDQRTKVVEIVRHPQYNESLSAQAGADIALLK
LEAPVPLSELIHPVSLPSASLDVPSGKTCWVTGWGVIGRGELLPWPLSLWEATVKVRSNVLCNQTCRR
RFPSNHTERFERLIKDDMLCAGDGNHGSWPGDNGGPLLCRRNCTWVQVEVVSWGKLCGLRGYPGMYTR
VTSYVSWIRQYVPPFPRRLE

| | | |
|---|---|---|
| NOV71e, CG59490-02 | SEQ ID NO: 1045 | 672 bp |

| | | |
|---|---|---|
| DNA Sequence | ORF Start: at 7 | ORF Stop: at 667 |

AGATCTTCACTGGGGGCTGCGACGTCTCGGCCAGGAGGCACCCCTGGCAGGGAGGAGTTGGAGGCTTG
CGCGTTTAGAGTGCAGGTGGGGCAGCTGAGGCTCTATGAGGACGACCAGCGGACGAAGGTGGTTGAGA
TCGTCCGTCACCCCCAGTACAACGAGAGCCTGTCTGCCCAGGGCGGTGCGGACATCGCCCTGCTGAAG
CTGGAGGCCCCGGTGCCGCTGTCTGAGCTCATCCACCCGGTCTCGCTCCCGTCTGCCTCCCTGGACGT
GCCCTCGGGGAAGACCTGCTGGGTGACCGGCTGGGGTGTCATTGGACGTGGAGAACTACTGCCCTGGC
CCCTCAGCTTGTGGGAGGCGACGGTGAAGGTCAGGAGCAACGTCCTCTGTAACCAGACCTGTCGCCGC
CGCTTTCCTTCCAACCACACTGAGCGGTTTGAGCGGCTCATCAAGGACGACATGCTGTGTGCCGGGGA
CGGGAACCACGGCTCCTGGCCAGGCGACAACGGGGGCCCCCTCCTGTGCAGGCGGAATTGCACCTGGG
TCCAGGTGGAGGTGGTGAGCTGGGGCAAACTCTGCGGCTATCGCGGCTATCCCGGCATGTACACCCGC
GTGACGAGCTACGTGTCCTGGATCCGCCAGTACGTCCCGCCGTTCCCCAGACGCCTCGAG

NOV71e, CG59490-02

| | | | |
|---|---|---|---|
| Protein Sequence | SEQ ID NO: 1046 | 220 aa | MW at 24484.8kD |

SLGAATSRPGGTPGREELEACAFRVQVGQLRLYEDDQRTKVVEIVRHPQYNESLSAQGGADIALLKLE
APVPLSELIHPVSLPSASLDVPSGKTCWVTGWGVIGRGELLPWPLSLWEATVKVRSNVLCNQTCRRRF
PSNHTERFERLIKDDMLCAGDGNHGSWPGDNGGPLLCRRNCTWVQVEVVSWGKLCGLRGYPGMYTRVT
SYVSWIRQYVPPFPRR

A ClustalW comparison of the above protein sequences yields the following sequence alignment shown in Table 71B.

TABLE 71B
Comparison of the NOV71 protein sequences.

| | |
|---|---|
| NOV71a | --SLGAATSRPGGTPGREELEACAFRVQVGQLRLYEDDQRTKVVEIVRHPQYNESLSAQG |
| NOV71b | RSSLGAATSRPGGTPGREELEACAFRVQVGQLRLYEDDQRTKVVEIVRHPQYNESLSAQG |
| NOV71c | RSSLGAATSRPGGTPGREELEACAFRVQVGQLRLYEDDQRTKVVEIVRHPQYNESLSAQG |
| NOV71d | RSSLGAATSRPGGTPGREELEACAFRVQVGQLRLYEDDQRTKVVEIVRHPQYNESLSAQG |
| NOV71e | --SLGAATSRPGGTPGREELEACAFRVQVGQLRLYEDDQRTKVVEIVRHPQYNESLSAQG |
| NOV71a | GADIALLKLEAPVPLSELIHPVSLPSASRDVPSGKTCWVTGWGVIGRGELLPWPLSLWEA |
| NOV71b | GADIALLKLEAPVPLSELIHPVSLPSASRDVPSGKTCWVTGWGVIGRGELLPWPLSLWEA |
| NOV71c | GADIALLKLEAPVPLSELIHPVSLPSASRDVPSGKTCWVTGWGVIGRGELLPWPLSLWEA |
| NOV71d | GADIALLKLEAPVPLSELIHPVSLPSASRDVPSGKTCWVTGWGVIGRGELLPWPLSLWEA |
| NOV71e | GADIALLKLEAPVPLSELIHPVSLPSASRDVPSGKTCWVTGWGVIGRGELLPWPLSLWEA |
| NOV71a | TVKVRSNVLCNQTCRRRFPSNHTERFERLIKDDMLCAGDGNHGSWPGDNGGPLLCRRNCT |
| NOV71b | TVKVRSNVLCNQTCRRRFPSNHTERFERLIKDDMLCAGDGNHGSWPGDNGGPLLCRRNCT |
| NOV71c | TVKVRSNVLCNQTCRRRFPSNHTERFERLIKDDMLCAGDGNHGSWPGDNGGPLLCRRNCT |
| NOV71d | TVKVRSNVLCNQTCRRRFPSNHTERFERLIKDDMLCAGDGNHGSWPGDNGGPLLCRRNCT |
| NOV71e | TVKVRSNVLCNQTCRRRFPSNHTERFERLIKDDMLCAGDGNHGSWPGDNGGPLLCRRNCT |
| NOV71a | WVQVEVVSWGKLCGLRGYPGMYTRVTSYVSWIRQYVPPFPRR-- |
| NOV71b | WVQVEVVSWGKLCGLRGYPGMYTRVTSYVSWIRQYVPPFPRRLE |
| NOV71c | WVQVEVVSWGKLCGLRGYPGMYTRVTSYVSWIRQYVPPFPRRLE |
| NOV71d | WVQVEVVSWGKLCGLRGYPGMYTRVTSYVSWIRQYVPPFPRRLE |
| NOV71e | WVQVEVVSWGKLCGLRGYPGMYTRVTSYVSWIRQYVPPFPRR-- |
| NOV71a | (SEQ ID NO: 1038) |
| NOV71b | (SEQ ID NO: 1040) |
| NOV71c | (SEQ ID NO: 1042) |
| NOV71d | (SEQ ID NO: 1044) |
| NOV71e | (SEQ ID NO: 1046) |

Further analysis of the NOV71a protein yielded the following properties shown in Table 71C.

TABLE 71C
Protein Sequence Properties NOV71a

| | |
|---|---|
| SignalP analysis: | No Known Signal Sequence Indicated |

PSORT II analysis:

| PSG: | a new signal peptide prediction method | | | |
|---|---|---|---|---|
| | N-region: | length 8; | pos.chg 1; | neg.chg 0 |
| | H-region: | length 6; | peak value | −8.72 |
| | PSG score: | −13.12 | | |

| | |
|---|---|
| GvH: | von Heijne's method for signal seq. recognition |
| | GvH score (threshold: −2.1): −7.58 |
| | possible cleavage site: between 14 and 15 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 4.88 (at 62) |
| | ALOM score: 4.88 (number of TMSs: 0) |

MITDISC: discrimination of mitochondrial targeting seq

| | | | |
|---|---|---|---|
| R content: | 2 | Hyd Moment (75): | 6.94 |
| Hyd Moment (95): | 7.43 | G content: | 4 |
| D/E content: | 1 | S/T content: | 4 |
| Score: −3.60 | | | |

Gavel: prediction of cleavage sites for mitochondrial preseq

R-2 motif at 18 SRP|GG

NUCDISC: discrimination of nuclear localization signals

pat4: none
pat7: none
bipartite: none
content of basic residues: 12.3%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
  39.1%: cytoplasmic
  30.4%: mitochondrial
  17.4%: nuclear
    8.7%: vesicles of secretory system
    4.3%: vacuolar
```

>> prediction for CG59490-01 is cyt (k = 23)

A search of the NOV71a protein against the Geneseq database, a proprietary database that contains sequences published in patents and patent publication, yielded several homologous proteins shown in Table 71D.

TABLE 71D
Geneseq Results for NOV71a

| | | NOV71a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE08591 | Human NOV12 protein - Homo | 1 . . . 220 | 220/220 (100%) | e−133 |
| | sapiens, 220 aa. [WO200161009- | 1 . . . 220 | 220/220 (100%) | |
| | A2, 23-AUG-2001] | | | |
| AAE14347 | Human protease PRTS-12 | 15 . . . 220 | 205/206 (99%) | e−123 |
| | protein - Homo sapiens, 262 aa. | 57 . . . 262 | 205/206 (99%) | |
| | [WO200183775-A2, 08-NOV- | | | |
| | 2001] | | | |
| AAE08590 | Human NOV11 protein - Homo | 16 . . . 220 | 204/205 (99%) | e−123 |
| | sapiens, 285 aa. [WO200161009- | 81 . . . 285 | 204/205 (99%) | |
| | A2, 23-AUG-2001] | | | |
| AAU82736 | Amino acid sequence of novel | 15 . . . 215 | 198/201 (98%) | e−118 |
| | human protease #35 - Homo | 211 . . . 411 | 198/201 (98%) | |
| | sapiens, 948 aa. [WO200200860- | | | |
| | A2, 03-JAN-2002] | | | |
| AAE08587 | Human NOV8 protein - Homo | 14 . . . 220 | 200/207 (96%) | e−118 |
| | sapiens, 290 aa. [WO200161009- | 84 . . . 290 | 200/207 (96%) | |
| | A2, 23-AUG-2001] | | | |

In a BLAST search of public sequence databases, the NOV71a protein was found to have homology to the proteins shown in the BLASTP data in Table 71E.

TABLE 71E
Public BLASTP Results for NOV71a

| | | NOV71a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAP21675 | Mast cell protease-11 - Mus | 16 . . . 217 | 118/202 (58%) | 2e−59 |
| | musculus (Mouse), 318 aa. | 86 . . . 284 | 142/202 (69%) | |
| AAA30855 | Mastin precursor - Canis sp, 251 | 16 . . . 218 | 110/203 (54%) | 9e−51 |
| | aa (fragment). | 53 . . . 249 | 127/203 (62%) | |
| P19236 | Mastocytoma protease precursor | 16 . . . 218 | 110/203 (54%) | 9e−51 |
| | (EC 3.4.21.—) - Canis | 71 . . . 267 | 127/203 (62%) | |
| | familiaris (Dog), 269 aa. | | | |
| Q8SQ44 | Tryptase precursor - Sus scrofa | 24 . . . 218 | 105/195 (53%) | 7e−49 |
| | (Pig), 277 aa. | 90 . . . 275 | 127/195 (64%) | |
| Q9XSM1 | Tryptase (EC 3.4.21.59) - Ovis | 14 . . . 218 | 89/205 (43%) | 1e−43 |
| | aries (Sheep), 273 aa. | 75 . . . 273 | 120/205 (58%) | |

PFam analysis indicates that the NOV71a protein contains the domains shown in the Table 71F.

TABLE 71F
Domain Analysis of NOV71a

| Pfam | NOV71a | Identities/Similarities | Expect |
|---|---|---|---|
| Domain | Match Region | for the Matched Region | Value |
| trypsin | 8 . . . 210 | 81/272 (30%) | 1.9e−32 |
| | | 152/272 (56%) | |

### Example 72

The NOV72 clone was analyzed, and the nucleotide and encoded polypeptide sequences are shown in Table 72A.

TABLE 72A
NOV72 Sequence Analysis

| | | |
|---|---|---|
| NOV72a, CG59693-01 | SEQ ID NO: 1047 | 972 bp |

| | | |
|---|---|---|
| DNA Sequence | ORF Start: ATG at 1 | ORF Stop: TAA at 970 |

ATGGATTCGAAATATCAGTGTGTGAAGCTGAATGATGGTCACTTCATGCCTGTCCTGGGATTTGGCAC
CTATGCGCCTGCAGAGGTTCCTAAAAGTAAAGCTTTAGAGGCCACCAAATTGGCAATTGAAGCTGGCT
TCCGCCATATTGATTCTGCTCATTTATACAATAATGAGGAGCAGGTTGGACTGGCCATCCGAAGCAAG
ATTGCAGATGGCAGTGTGAAGAGAGAAGACATATTCTACACTTCAAAGCTTTGGTGCAATTCCCATCG
ACCAGAGTTGGTCCGACCAGCCTTGGAAAGGTCACTGAAAAATCTTCAATTGGATTATGTTGACCTCT
ACCTTATTCATTTTCCAGTGTCTGTAAAGCCAGGTGAGGAAGTGATCCCAAAAGATGAAAATGGAAAA
ATACTATTTGACACAGTGGATCTCTGTGCCACGTGGGAGGCCGTGGAGAAGTGTAAAGATGCAGGATT
GGCCAAGTCCATCGGGGTGTCCAACTTCAACCGCAGGCAGCTGGAGATGATCCTCAACAAGCCAGGGC
TCAAGTACAAGCCTGTCTGCAACCAGGTGGAATGTCATCCTTACTTCAACCAGAGAAAACTGCTGGAT
TTCTGCAAGTCAAAAGACATTGTTCTGGTTGCCTATAGTGCTCTGGGATCCCACCGAGAAGAACCATG
GGTGGACCCGAACTCCCCGGTGCTCTTGGAGGACCCAGTCCTTTGTGCCTTGGCAAAAAAGCACAAGC
GAACCCCAGCCCTGATTGCCCTGCGCTACCAGCTACAGCGTGGGGTTGTGGTCCTGGCCAAGAGCTAC
AATGAGCAGCGCATCAGACAGAACGTGCAGGTGTTTGAATTCCAGTTGACTTCAGAGGAGATGAAAGC
CATAGATGGCCTAAACAGAAATGTGCGATATTTGACCCTTGATATTTTTGCTGGCCCCCCTAATTATC
CATTTTCTGATGAATATTAA

NOV72a, CG59693-01

| | | | |
|---|---|---|---|
| Protein Sequence | SEQ ID NO: 1048 | 323 aa | MW at 36787.9kD |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV72b, CG59693-03 | SEQ ID NO: 1049 | 972 bp |

| | | |
|---|---|---|
| DNA Sequence | ORF Start: ATG at 1 | ORF Stop: TAA at 970 |

ATGGATTCGAAATATCAGTGTGTGAAGCTGAATGATGGTCACTTCATGCCTGTCCTGGGATTTGGCAC
CTATGCGCCTGCAGAGGTTCCTAAAAGTAAAGCTTTAGAGGCCACCAAATTGGCAATTGAAGCTGGCT
TCCGCCATATTGATTCTGCTCATTTATACAATAATGAGGAGCAGGTTGGACTGGCCATCCGAAGCAAG
ATTGCAGATGGCAGTGTGAAGAGAGAAGACATATTCTACACTTCAAAGCTTTGGTGCAATTCCCATCG
ACCAGAGTTGGTCCGACCAGCCTTGGAAAGGTCACTGAAAAATCTTCAATTGGATTATGTTGACCTCT
ACCTTATTCATTTTCCAGTGTCTGTAAAGCCAGGTGAGGAAGTGATCCCAAAAGATGAAAATGGAAAA
ATACTATTTGACACAGTGGATCTCTGTGCCACGTGGGAGGCCGTGGAGAAGTGTAAAGATGCAGGATT
GGCCAAGTCCATCGGGGTGTCCAACTTCAACCGCAGGCAGCTGGAGATGATCCTCAACAAGCCAGGGC
TCAAGTACAAGCCTGTCTGCAACCAGGTGGAATGTCATCCTTACTTCAACCAGAGAAAACTGCTGGAT
TTCTGCAAGTCAAAAGACATTGTTCTGGTTGCCTATAGTGCTCTGGGATCCCACCGAGAAGAACCATG
GGTGGACCCGAACTCCCCGGTGCTCTTGGAGGACCCAGTCCTTTGTGCCTTGGCAAAAAAGCACAAGC
GAACCCCAGCCCTGATTGCCCTGCGCTACCAGCTACAGCGTGGGGTTGTGGTCCTGGCCAAGAGATAC
AATGAGCAGCGCATCAGACAGAACGTGCAGGTGTTTGAATTCCAGTTGACTTCAGAGGAGATGAAAGC
CATAGATGGCCTAAACAGAAATGTGCGATATTTGACCCTTGATATTTTTGCTGGCCCCCCTAATTATC
CATTTTCTGATGAATATTAA

NOV72b, CG59693-03

| | | | |
|---|---|---|---|
| Protein Sequence | SEQ ID NO: 1050 | 323 aa | MW at 36857.0kD |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKRY
NEQRIRQNVQVFEFQLTSEEMKAIDGLNRNVRYLTLDIFAGPPNYPFSDEY

| | | |
|---|---|---|
| NOV72c, 277637252 | SEQ ID NO: 1051 | 1001 bp |

| | | |
|---|---|---|
| DNA Sequence | ORF Start: at 8 | ORF Stop: TAA at 989 |

CATCTAGGCCACCATGGCCATGGATTCGAAATATCAGTGTGTGAAGCTGAATGATGGTCACTTCATGC
CTGTCCTGGGATTTGGCACCTATGCGCCTGCAGAGGTTCCTAAAAGTAAAGCTTTAGAGGCCACCAAA
TTGGCAATTGAAGCTGGCTTCCGCCATATTGATTCTGCTCATTTATACAATAATGAGGAGCAGGTTGG
ACTGGCCATCCGAAGCAAGATTGCAGATGGCAGTGTGAAGAGAGAAGACATATTCTACACTTCAAAGC
TTTGGTGCAATTCCCATCGACCAGAGTTGGTCCGACCAGCCTTGGAAAGGTCACTGAAAAATCTTCAA
TTGGATTATGTTGACCTCTACCTTATTCATTTTCCAGTGTCTGTAAAGCCAGGTGAGGAAGTGATCCC
AAAAGATGAAAATGGAAAAATACTATTTGACACAGTGGATCTCTGTGCCACGTGGGAGGCCGTGGAGA
AGTGTAAAGATGCAGGATTGGCCAAGTCCATCGGGGTGTCCAACTTCAACCGCAGGCAGCTGGAGATG
ATCCTCAACAAGCCAGGGCTCAAGTACAAGCCTGTCTGCAACCAGGTGGAATGTCATCCTTACTTCAA
CCAGAGAAAAGTGCTGGATTTCTGCAAGTCAAAAGACATTGTTCTGGTTGCCTATAGTGCTCTGGGAT
CCCACCGAGAAGAACCATGGGTGGACCCGAACTCCCCGGTGCTCTTGGAGGACCCAGTCCTTTGTGCC
TTGGCAAAAAGCACAAGCGAACCCCAGCCCTGATTGCCCTGCGCTACCCAGCTACAGCGTGGGGTTGT
GGTCCTGGCCAAGAGCTACAATGAGCAGCGCATCAGACAGAACGTGCAGGTGTTTGAATTCCAGTTGA
CTTCAGAGGAGATGAAAGCCATAGATGGCCTAAACAGAAATGTGCGATATTTGACCCTTGATATTTTT
GCTGGCCCCCCTAATTATCCATTTTCTGATGAATATTAAACGCGTGATC

NOV72c, 277637252

| | | | |
|---|---|---|---|
| Protein Sequence | SEQ ID NO: 1052 | 327 aa | MW at 37162.4kD |

ATMAMDSKYQCVKLNDGHFMPVLGFGTYAPAEVPKSKALEATKLAIEAGFRHIDSAHLYNNEEQVGLA
IRSKIADGSVKREDIFYTSKLWCNSHRPELVRPALERSLKNLQLDYVDLYLIHFPVSVKPGEEVIPKD
ENGKILFDTVDLCATWEAVEKCKDAGLAKSIGVSNFNRRQLEMILNKPGLKYKPVCNQVECHPYFNQR
KLLDFCKSKDIVLVAYSALGSHREEPWVDPNSPVLLEDPVLCALAKKHKRTPALIALRYQLQRGVVVL
AKSYNEQRIRQNVQVFEFQLTSEEMKAIDGLNRNVRYLTLDIFAGPPNYPFSDEY

| | | |
|---|---|---|
| NOV72d, CG59693-02 | SEQ ID NO: 1053 | 983 bp |

| | | |
|---|---|---|
| DNA Sequence | ORF Start: ATG at 30 | ORF Stop: TAA at 981 |

ATGGATTCGATATCAGTGTGTGAAGCTGAATGATGGTCACTTCGTGCCTGTCCTGGGATTTGGCACCT
ATGCGCCTGCAGAGGTTACTCCCCCAGGTTCCTAAAAGTAAAGCTTTAGAGGCCACCAAATTGGCAAT
TGAAGCTGGCTTCCGCCATATTGATTCTGCTCATTTATACAATAATGAGGAGCAGGTTGGACTGGCCA
TCCGAAGCAAGATTGCAGATGGCAGTGTGAAGAGAGAAGACATATTCTACACTTCAAAGCTTTGGTGC
AATTCCCATCGACCAGAGTTGGTCCGACCAGCCTTGGAAAGGTCACTGAAAAATCTTCAATTGGATTA
TGTTGACCTCTACCTTATTCATTTTCCAGTGTCTGTAAAGCCAGGTGAGGAAGTGATCCCAAAAGATG
AAGTGGAAAAATACTATTTGACACAGTGGATCTCTGTGCCACGTGCCAAGGCCGTGGAGAAGTGTAAA
GATGCAGGATTGGCCAAGTCCATCGGGGTGTCCAACTTCAACCGCAGGCAGCTGGAGATGATCCTCAA
CAAGCCAGGGCTCAAGTACAAGCCTGTCTGCAACCAGGTGGAATGTCATCCTTACTTCAACCAGAGAA
AACTGCTGGATTTCTGCAAGTCAAAAGACATTGTTCTGGTTGCCTATAGTGCTCTGGGATCCCACCGA
GAAGAACCATGGGTGGACCCGAACTCCCCGGTGCTCTTGGAGGACCCAGTCCTTTGTGCCTTGGCAAA
AAAGCACAAGCGAACCCCAGCCCTGGTTGCCCTGCGCTACCAGCTACAGCGTGGGGTTGTGGTCCTGG
CCAAGAGCTACAATGAGCAGCGCATCAGACAGAACGTGCAGGTGTTTGAATTCCAGTTGACTTCAGAG
GAGATGAAAGCCATAGATGGCCTAAACAGAAATGTGCGATATTTGACCCTTGATATTTTTGCTGGCCC
CCCTAATTATCCATTTTCTGATGAATATTAA

NOV72d, CG59693-02

| | | | |
|---|---|---|---|
| Protein Sequence | SEQ ID NO: 1054 | 317 aa | MW at 36217.5kD |

MMVTSCLSWDLAPMRLQRLLPQVPKSKALEATKLAIEAGRFHIDSAHLYNNEEQVGLAIRSKIADGSV
KREDIFYTSKLWCNSHRPELVRPALERSLKNLQLDYVDLYLIHFPVSVKPGEEVIPKDESGKILFDTV
DLCATWEAVEKCKDAGLAKSIGVSNFNRRQLEMILNKPGIKYKPVCNQVECHPYFNQRKLLDFCKSKD
IVLVAYSALGSHREEPWVDPNSPVLLEDPVLCALAKKHKRTPALVALRYQLQRGVVVIAKSYNEQRIR
QNVQVFEFQLTSEEMKAIDGLNRNVRYLTLDIFAGPPNYPFSDEY

| | | |
|---|---|---|
| NOV72e, CG59693-04 | SEQ ID NO: 1055 | 994 bp |

| | | |
|---|---|---|
| DNA Sequence | ORF Start: ATG at 16 | ORF Stop: at 979 |

GCCAGATCTCCCACCATGGATTCGAAATATCAGTGTGTGAAGCTGAATGATGGTCACTTCATGCCTGT
CCTGGGATTTGGCACCTATGCGCCTGCAGAGGTTCCTAAAAGTAAAGCTTTAGAGGCCACCAAATTGG
CAATTGAAGCTGGCTTCCGCCATATTGATTCTGCTCATTTATACAATAATGAGGAGCAGGTTGGACTG
GCCATCCGAAGCAAGATTGCAGATGGCAGTGTGAAGAGAGAAGACATATTCTACACTTCAAAGCTTTG
GTGCAATTCCCATCGACCAGAGTTGGTCCGACCAGCCTTGGAAAGGTCACTGAAAAATCTTCAATTGG
ATTATGTTGACCTCTACCTTATTCATTTTCCAGTGTCTGTAAAGCCAGGTGAGGAAGTGATCCCAAAA
GATGAAAATGGAAAAATACTATTTGACACAGTGGATCTCTGTGCCACGTGGGAGGCCGTGGAGAAGTG
TAAAGATGCAGGATTGGCCAAGTCCATCGGGGTGTCCAACTTCAACCGCAGGCAGCTGGAGATGATCC
TCAACAAGCCAGGGCTCAAGTACAAGCCTGTCTGCAACCAGGTGGAATGTCATCCTTACTTCAACCAG
AGAAAACTGCTGGATTTCTGCAAGTCAAAAGACATTGTTCTGGTTGCCTATAGTGCTCTGGGATCCCA
CCGAGAAGAACCATGGGTGGACCCGAACTCCCCGGTGCTCTTGGAGGACCCAGTCCTTTGTGCCTTGG
CAAAAAAGCACAAGCGAACCCCAGCCCTGATTGCCCTGCGCTACCAGCTACAGCGTGGGGTTGTGGTC
CTGGCCAAGAGCTACAATGAGCAGCGCATCAGACAGAACGTGCAGGTGTTTGAATTCCAGTTGACTTC
AGAGGAGATGAAAGCCATAGATGGCCTAAACAGAAATGTGCGATATTTGACCCTTGATATTTTTGCTG
GCCCCCCTAATTATCCATTTTCTGATGAATATGTCGAGGGTG

NOV72e, CG59693-04

| | | | |
|---|---|---|---|
| Protein Sequence | SEQ ID NO: 1056 | 321 aa | MW at 36495.6kD |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVIVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSD

| | | |
|---|---|---|
| NOV72f, CG59693-05 | SEQ ID NO: 1057 | 1219 bp |

| | | |
|---|---|---|
| DNA Sequence | ORF Start: ATG at 24 | ORF Stop: TAA at 993 |

TGCTAACCAGGCCAGTGACAGAAATGGATTCGAAATACCAGTGTGTGAAGCTGAATGATGGTCACTTC
ATGCCTGTCCTGGGATTTGGCACCTATGCGCCTGCAGAGGTTCCTAAAAGTAAAGCTCTAGAGGCCGT
CAAATTGGCAATAGAAGCCGGGTTCCACCATATTGATTCTGCACATGTTTACAATAATGAGGAGCAGG
TTGGACTGGCCATCCGAAGCAAGATTGCAGATGGCAGTGTGAAGAGAGAAGACATATTCTACACTTCA
AAGCTTTGGAGCAATTCCCATCGACCAGAGTTGGTCCGACCAGCCTTGGAAAGGTCACTGAAAAATCT
TCAATTGGACTATGTTGACCTCTATCTTATTCATTTTCCAGTGTCTGTAAAGCCAGGTGAGGAAGTGA
TCCCAAAAGATGAAAATGGAAAAATACTATTTGACACAGTGGATCTCTGTGCCACGTGGGAGGCCATG
GAGAAGTGTAAAGATGCAGGATTGGCCAAGTCCATCGGGGTGTCCAACTTCAACCACAGGCTGCTGGA
GATGATCCTCAACAAGCCAGGGCTCAAGTACAAGCCTGTCTGCAACCAGGTGGAATGTCATCCTTACT
TCAACCAGAGAAAACTGCTGGATTTCTGCAAGTCAAAAGACATTGTTCTGGTTGCCTATAGTGCTCTG
GGATCCCATCGAGAAGAACCATGGGTGGACCCGAACTCCCCGGTGCTCTTGGAGGACCCAGTCCTTTG
TGCCTTGGCAAAAAAGCACAAGCGAACCCCAGCCCTGATTGCCCTGCGCTACCAGCTGCAGCGTGGGG
TTGTGGTCCTGGCCAAGAGCTACAATGAGCAGCGCATCAGACAGAACGTGCAGGTGTTTGAATTCCAG
TTGACTTCAGAGGAGATGAAAGCCATAGATGGCCTAAACAGAAATGTGCGATATTTGACCCTTGATAT
TTTTGCTGGCCCCCCTAATTATCCATTTTCTGATGAATATTAACATGGAGGGCATTGCATGAGGTCTG
CCAGAAGGCCCTGCGTGTGGATGGTGACACAGAGGATGGCTCTATGCTGGTGACTGGACACATCGCCT
CTGGTTAAATCTCTCCTGCTTGGCGACTTCAGTAAGCTACAGCTAAGCCCATCGGCCGGAAAAGAAAG
ACAATAATTTTGTTTTTTCATTTTGAAAAAATTAAATGCTCTCTCCTAAAGATTCTTCACCTA

NOV72f, CG59693-05

| | | | |
|---|---|---|---|
| Protein Sequence | SEQ ID NO: 1058 | 323 aa | MW at 36734.9kD |

MDSKYQCVKLNDGHFNPVLGFGTYAPAEVPKSKALEAVKLAIEAGFHHIDSAHVYNNEEQVGLAIRSK
IADGSVKREDIFYTSKLWSNSHRPELVRPALERSLKNLQLDYVDLYLIHFPVSVKPGEEVIPKDENGK
ILFDTVDLCATWEAMEKCKDAGLAKSIGVSNFNHRLLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV72g, CG59693-06 | SEQ ID NO: 1059 | 1001 bp |

| | | |
|---|---|---|
| DNA Sequence | ORF Start: at 11 | ORF Stop: at 983 |

CATCTAGGCCACCATGGCCATGGATTCGAAATATCAGTGTGTGAAGCTGAATGATGGTCACTTCATGC
CTGTCCTGGGATTTGGCACCTATGCGCCTGCAGAGGTTCCTAAAAGTAAAGCTTTAGAGGCCACCAAA
TTGGCAATTGAAGCTGGCTTCCGCCATATTGATTCTGCTCATTTATACAATAATGAGGAGCAGGTTGG
ACTGGCCATCCGAAGCAAGATTGCAGATGGCAGTGTGAAGAGAGAAGACATATTCTACACTTCAAAGC
TTTGGTGCAATTCCCATCGACCAGAGTTGGTCCGACCAGCCTTGGAAAGGTCACTGAAAAATCTTCAA
TTGGATTATGTTGACCTCTACCTTATTCATTTTCCAGTGTCTGTAAAGCCAGGTGAGGAAGTGATCCC
AAAAGATGAAAATGGAAAAATACTATTTGACACAGTGGATCTCTGTGCCACGTGGGAGGCCGTGGAGA
AGTGTAAAGATGCAGGATTGGCCAAGTCCATCGGGGTGTCCAACTTCAACCGCAGGCAGCTGGAGATG
ATCCTCAACAAGCCAGGGCTCAAGTACAAGCCTGTCTGCAACCAGGTGGAATGTCATCCTTACTTCAA
CCAGAGAAAACTGCTGGATTTCTGCAAGTCAAAAGACATTGTTCTGGTTGCCTATAGTGCTCTGGGAT
CCCACCGAGAAGAACCATGGGTGGACCCGAACTCCCCGGTGCTCTTGGAGGACCCAGTCCTTTGTGCC
TTGGCAAAAAAGCACAAGCGAACCCCAGCCCTGATTGCCCTGCGCTACCAGCTACAGCGTGGGGTTGT
GGTCCTGGCCAAGAGCTACAATGAGCAGCGCATCAGACAGAACGTGCAGGTGTTTGAATTCCAGTTGA
CTTCAGAGGAGATGAAAGCCATAGATGGCCTAAACAGAAATGTGCGATATTTGACCCTTGATATTTTT
GCTGGCCCCCCTAATTATCCATTTTCTGATGAATATTAAACGCGTGATC

NOV72g, CG59693-06

| | | | |
|---|---|---|---|
| Protein Sequence | SEQ ID NO: 1060 | 324 aa | MW at 36799.0kD |

TMAMDSKYQCVKLNDGHFMPVLGFGTYAPAEVPKSKALEATKLAIEAGFRHIDSAHLYNNEEQVGLAI
RSKIADGSVKREDIFYTSKLWCNSHRPELVRPALERSLKNLQLDYVDLYLIHFPVSVKPGEEVIPKDE
NGKILFDTVDLCATWEAVEKCKDAGLAKSIGVSNFNRRQLEMILNKPGLKYKPVCNQVECHPYFNQRK
LLDFCKSKDIVLVAYSALGSHREEPWVDPNSPVLLEDPVLCALAKKHKRTPALIALRYQLQRGVVVLA
KSYNEQRIRQNVQVFEFQLTSEEMKAIDGLNRNVRYLTLDIFAGPPNYPFSD

| | | |
|---|---|---|
| NOV72h, CG59693-07 | SEQ ID NO: 1061 | 1012 bp |

| | | |
|---|---|---|
| DNA Sequence | ORF Start: at 1 | ORF Stop: at 1012 |

GCCGGTACCACCATGGGCCACCATCACCACCATCACGATTCGAAATATCAGTGTGTGAAGCTGAATGA
TGGTCACTTCATGCCTGTCCTGGGATTTGGCACCTATGCGCCTGCAGAGGTTCCTAAAAGTAAAGCTT
TAGAGGCCACCAAATTGGCAATTGAAGCTGGCTTCCGCCATATTGATTCTGCTCATTTATACAATAAT
GAGGAGCAGGTTGGACTGGCCATCCGAAGCAAGATTGCAGATGGCAGTGTGAAGAGAGAAGACATATT
CTACACTTCAAAGCTTTGGTGCAATTCCCATCGACCAGAGTTGGTCCGACCAGCCTTGGAAAGGTCAC
TGAAAAATCTTCAATTGGATTATGTTGACCTCTACCTTATTCATTTTCCAGTGTCTGTAAAGCCAGGT
GAGGAAGTGATCCCAAAAGATGAAAATGGAAAAATACTATTTGACACAGTGGATCTCTGTGCCACGTG
GGAGGCCGTGGAGAAGTGTAAAGATGCAGGATTGGCCAAGTCCATCGGGGTGTCCAACTTCAACCGCA
GGCAGCTGGAGATGATCCTCAACAAGCCAGGGCTCAAGTACAAGCCTGTCTGCAACCAGGTGGAATGT
CATCCTTACTTCAACCAGAGAAAACTGCTGGATTTCTGCAAGTCAAAAGACATTGTTCTGGTTGCCTA
TAGTGCTCTGGGATCCCACCGAGAAGAACCATGGGTGGACCCGAACTCCCCGGTGCTCTTGGAGGACC
CAGTCCTTTGTGCCTTGGCAAAAAAGCACAAGCGAACCCCAGCCCTGATTGCCCTGCGCTACCAGCTA
CAGCGTGGGGTTGTGGTCCTGGCCAAGAGCTACAATGAGCAGCGCATCAGACAGAACGTGCAGGTGTT
TGAATTCCAGTTGACTTCAGAGGAGATGAAAGCCATAGATGGCCTAAACAGAAATGTGCGATATTTGA
CCCTTGATATTTTTGCTGGCCCCCCTAATTATCCATTTTCTGATGAATATCTCGAGGGTG

NOV72h, CG59693-07

| | | | |
|---|---|---|---|
| Protein Sequence | SEQ ID NO: 1062 | 337 aa | MW at 38297.5kD |

AGTTMGHHHHHHDSKYQCVKLNDGHFMPVLGFGTYAPAEVPKSKALEATKLAIEAGFRHIDSAHLYNN
EEQVGLAIRSKIADGSVKREDIFYTSKLWCNSHRPELVRPALERSLKNLQLDYVDLYLIHFPVSVKPG
EEVIPKDENGKILFDTVDLCATWEAVEKCKDAGLAKSIGVSNFNRRQLEMILNKPGLKYKPVCNQVEC
HPYFNQRKLLDFCKSKDIVLVAYSALGSHREEPWVDPNSPVLLEDPVLCALAKKHKRTPALIALRYQL
QRGVVVLAKSYNEQRIRQNVQVFEFQLTSEEMKAIDGLNRNVRYLTLDIFAGPPNYPFSDEYLEG

| | | |
|---|---|---|
| NOV72i, CG59693-08 | SEQ ID NO: 1063 | 1225 bp |

| | | |
|---|---|---|
| DNA Sequence | ORF Start: ATG at 24 | ORF Stop: TAA at 993 |

TGCTAACCAGGCCAGTGACAGAAATGGATTCGAAATACCAGTGTGTGAAGCTGAATGATGGTCACTTC
ATGCCTGTCCTGGGATTTGGCACCTATGCGCCTGCAGAGGTTCCTAAAAGTAAAGCTCTAGAGGCCGT
CAAATTGGCAATAGAAGCCGGGTTCCACCATATTGATTCTGCACATGTTTACAATAATGAGGAGCAGG
TTGGACTGGCCATCCGAAGCAAGATTGCAGATGGCAGTGTGAAGAGAGAAGACATATTCTACACTTCA
AAGCTTTGGAGCAATTCCCATCGACCAGAGTTGGTCCGACCAGCCTTGGAAAGGTCACTGAAAAATCT
TCAATTGGACTATGTTGACCTCTATCTTATTCATTTTCCAGTGTCTGTAAAGCCAGGTGAGGAAGTGA
TCCCAAAAGATGAAAATGGAAAAATACTATTTGACACAGTGGATCTCTGTGCCACGTGGGAGGCCATG
GAGAAGTGTAAAGATGCAGGATTGGCCAAGTCCATCGGGGTGTCCAACTTCAACCACAGGCTGCTGGA
GATGATCCTCAACAAGCCAGGGCTCAAGTACAAGCCTGTCTGCAACCAGGTGGAATGTCATCCTTACT
TCAACCAGAGAAAACTGCTGGATTTCTGCAAGTCAAAAGACATTGTTCTGGTTGCCTATAGTGCTCTG
GGATCCCATCGAGAAGAACCATGGGTGGACCCGAACTCCCCGGTGCTCTTGGAGGACCCAGTCCTTTG
TGCCTTGGCAAAAAAGCACAAGCGAACCCCAGCCCTGATTGCCCTGCGCTACCAGCTGCAGCGTGGGG
TTGTGGTCCTGGCCAAGAGCTACAATGAGCAGCGCATCAGACAGAACGTGCAGGTGTTTGAATTCCAG
TTGACTTCAGAGGAGATGAAAGCCATAGATGGCCTAAACAGAAATGTGCGATATTTGACCCTTGATAT
TTTTGCTGGCCCCCCTAATTATCCATTTTCTGATGAATATTAACATGGAGGGCATTGCATGAGGTCTG
CCAGAAGGCCCTGCGTGTGGATGGTGACACAGAGGATGGCTCTATGCTGGTGACTGGACACATCGCCT
CTGGTTAAATCTCTCCTGCTTGGCGACTTCAGTAAGCTACAGCTAAGCCCATCGGCCGGAAAAGAAAG
ACAATAATTTTGTTTTTTCATTTTGAAAAAATTAAATGCTCTCTCCTAAAGATTCTTCACCTAAAAAA
A

NOV72i, CG59693-08

| | | | |
|---|---|---|---|
| Protein Sequence | SEQ ID NO: 1064 | 323 aa | MW at 36734.9kD |

MDSKYQCVKLNDGHFMPVLGFGTYAPAEVPKSKALEAVKLAIEAGFHHIDSAHVYNNEEQVGLAIRSK
IADGSVKREDIFYTSKLWSNSHRPELVRPALERSLKNLQLDYVDLTLIHFPVSVKPGEEVIPKDENGK
ILFDTVDLCATWEAMEKCKDAGLAKSIGVSNFNHRLLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV72j, CG59693-09 | SEQ ID NO: 1065 | 996 bp |

| | | |
|---|---|---|
| DNA Sequence | ORF Start: ATG at 16 | ORF Stop: TAG at 985 |

CACCGCGGCCGCACCATGGATTCGAAATATCAGTGTGTGAAGCTGAATGATGGTCACTTCATGCCTGT
CCTGGGATTTGGCACCTATGCGCCTGCAGAGGTTCCTAAAAGTAAAGCTTTAGAGGCCACCAAATTGG
CAATTGAAGCTGGCTTCCGCCATATTGATTCTGCTCATTTATACAATAATGAGGAGCAGGTTGGACTG
GCCATCCGAAGCAAGATTGCAGATGGCAGTGTGAAGAGAGAAGACATATTCTACACTTCAAAGCTTTG
GTGCAATTCCCATCGACCAGAGTTGGTCCGACCAGCCTTGGAAAGGTCACTGAAAAATCTTCAATTGG
ATTATGTTGACCTCTACCTTATTCATTTTCCAGTGTCTGTAAAGCCAGGTGAGGAAGTGATCCCAAAA
GATGAAAATGGAAAAATACTATTTGACACAGTGGATCTCTGTGCCACGTGGGAGGCCGTGGAGAAGTG
TAAAGATGCAGGATTGGCCAAGTCCATCGGGGTGTCCAACTTCAACCGCAGGCAGCTGGAGATGATCC
TCAACAAGCCAGGGCTCAAGTACAAGCCTGTCTGCAACCAGGTGGAATGTCATCCTTACTTCAACCAG
AGAAAACTGCTGGATTTCTGCAAGTCAAAAGACATTGTTCTGGTTGCCTATAGTGCTCTGGGATCCCA
CCGAGAAGAACCATGGGTGGACCCGAACTCCCCGGTGCTCTTGGAGGACCCAGTCCTTTGTGCCTTGG
CAAAAAAGCACAAGCGAACCCCAGCCCTGATTGCCCTGCGCTACCAGCTACAGCGTGGGGTTGTGGTC
CTGGCCAAGAGCTACAATGAGCAGCGCATCAGACAGAACGTGCAGGTGTTTGAATTCCAGTTGACTTC
AGAGGAGATGAAAGCCATAGATGGCCTAAACAGAAATGTGCGATATTTGACCCTTGATATTTTTGCTG
GCCCCCCTAATTATCCATTTTCTGATGAATATTAGGTCGACGGC

NOV72j, CG59693-09

| | | | |
|---|---|---|---|
| Protein Sequence | SEQ ID NO: 1066 | 323 aa | MW at 36787.9kD |

MDSKYQCVKLNDGHFMPVLGFGTYAPAEVPKSKALEATKLAIEAGFRHIDSAHLYNEEQVGLAIRSK
IADGSVKREDIFYTSKLWNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

A ClustalW comparison of the above protein sequences yields the following sequence alignment shown in Table 72B.

TABLE 72B
Comparison of the NOV72 protein sequences.

| | |
|---|---|
| NOV72a | -----------MDSKYQCVKLNDGHFNPVLGFGTYAPAEVPKSKALEATKLAIEAGFRHI |
| NOV72b | -----------MDSKYQCVKLNDGHFMPVLGFGTYAPAEVPKSKALEATKLAIEAGFRHI |
| NOV72c | -------ATMAMDSKYQCVKLNDGHFMPVLGFGTYAPAEVPKSKALEATKLAIEAGFRHI |
| NOV72d | ----------------MMVTSCLSWDLAPMRLQRLLP-QVPKSKALEATKLAIEAGFRHI |
| NOV72e | -----------MDSKYQCVKLNDGHFNPVLGFGTYAPAEVPKSKALEATKLAIEAGFRHI |
| NOV72f | -----------MDSKYQCVKLNDGHFMPVLGFGTYAPAEVPKSKALEATKLAIEAGFHHI |
| NOV72g | --------TMAMDSKYQCVKLNDGHFMPVLGFGTYAPAEVPKSKALEATKLAIEAGFRHI |
| NOV72h | AGTTMGHHHHHHDSKYQCVKLNDGHFMPVLGFGTYAPAEVPKSKALEATKLAIEAGFRHI |
| NOV72i | -----------MDSKYQCVKLNDGHFMPVLGFGTYAPAEVPKSKALEAVKLAIEAGFHHI |
| NOV72j | -----------MDSKYQCVKLNDGHFMPVLGFGTYAPAEVPKSKALEATKLAIEAGFRHI |
| NOV72a | DSAHLYNNEEQVGLAIRSKIADGSVKREDIFYTSKLWCNSHRPELVRPALERSLKNLQLD |
| NOV72b | DSAHLYNNEEQVGLAIRSKIADGSVKREDIFYTSKLWCNSHRPELVRPALERSLKNLQLD |
| NOV72c | DSAHLYNNEEQVGLAIRSKIADGSVKREDIFYTSKLWCNSHRPELVRPALERSLKNLQLD |
| NOV72d | DSAHLYNNEEQVGLAIRSKIADGSVKREDIFYTSKLWCNSHRPELVRPALERSLKNLQLD |
| NOV72e | DSAHLYNNEEQVGLAIRSKIADGSVKREDIFYTSKLWCNSHRPELVRPALERSLKNLQLD |
| NOV72f | DSAHLYNNEEQVGLAIRSKIADGSVKREDIFYTSKLWCNSHRPELVRPALERSLKNLQLD |
| NOV72g | DSAHLYNNEEQVGLAIRSKIADGSVKREDIFYTSKLWCNSHRPELVRPALERSLKNLQLD |
| NOV72h | DSAHLYNNEEQVGLAIRSKIADGSVKREDIFYTSKLWCNSHRPELVRPALERSLKNLQLD |
| NOV72i | DSAHVYNNEEQVGLAIRSKIADGSVKREDIFYTSKLWCNSHRPELVRPALERSLKNLQLD |
| NOV72j | DSAHLYNNEEQVGLAIRSKIADGSVKREDIFYTSKLWCNSHRPELVRPALERSLKNLQLD |
| NOV72a | YVDLYLIHFPVSVKPGEEVIPKDENGKILFDTVDLCATWEAVEKCKDAGLAKSIGVSNFN |
| NOV72b | YVDLYLIHFPVSVKPGEEVIPKDENGKILFDTVDLCATWEAVEKCKDAGLAKSIGVSNFN |
| NOV72c | YVDLYLIHFPVSVKPGEEVIPKDENGKILFDTVDLCATWEAVEKCKDAGLAKSIGVSNFN |
| NOV72d | YVDLYLIHFPVSVKPGEEVIPKDENGKILFDTVDLCATWEAVEKCKDAGLAKSIGVSNFN |
| NOV72e | YVDLYLIHFPVSVKPGEEVIPKDENGKILFDTVDLCATWEAVEKCKDAGLAKSIGVSNFN |
| NOV72f | YVDLYLIHFPVSVKPGEEVIPKDENGKILFDTVDLCATWEAMEKCKDAGLAKSIGVSNFN |
| NOV72g | YVDLYLIHFPVSVKPGEEVIPKDENGKILFDTVDLCATWEAVEKCKDAGLAKSIGVSNFN |
| NOV72h | YVDLYLIHFPVSVKPGEEVIPKDENGKILFDTVDLCATWEAVEKCKDAGLAKSIGVSNFN |
| NOV72i | YVDLYLIHFPVSVKPGEEVIPKDENGKILFDTVDLCATWEAMEKCKDAGLAKSIGVSNFN |
| NOV72j | YVDLYLIHFPVSVKPGEEVIPKDENGKILFDTVDLCATWEAVEKCKDAGLAKSIGVSNFN |
| NOV72a | RRQLEMILNKPGLKYKPVCNQVECHPYFNQRKLLDFCKSKDIVLVAYSALGSHREEPWVD |
| NOV72b | RRQLEMILNKPGLKYKPVCNQVECHPYFNQRKLLDFCKSKDIVLVAYSALGSHREEPWVD |
| NOV72c | RRQLEMILNKPGLKYKPVCNQVECHPYFNQRKLLDFCKSKDIVLVAYSALGSHREEPWVD |
| NOV72d | RRQLEMILNKPGLKYKPVCNQVECHPYFNQRKLLDFCKSKDIVLVAYSALGSHREEPWVD |
| NOV72e | RRQLEMILNKPGLKYKPVCNQVECHPYFNQRKLLDFCKSKDIVLVAYSALGSHREEPWVD |
| NOV72f | HRLLEMILNKPGLKYKPVCNQVECHPYFNQRKLLDFCKSKDIVLVAYSALGSHREEPWVD |
| NOV72g | RRQLEMILNKPGLKYKPVCNQVECHPYFNQRKLLDFCKSKDIVLVAYSALGSHREEPWVD |
| NOV72h | RRQLEMILNKPGLKYKPVCNQVECHPYFNQRKLLDFCKSKDIVLVAYSALGSHREEPWVD |
| NOV72i | HRLLEMILNKPGLKYKPVCNQVECHPYFNQRKLLDFCKSKDIVLVAYSALGSHREEPWVD |
| NOV72j | RRQLEMILNKPGLKYKPVCNQVECHPYFNQRKLLDFCKSKDIVLVAYSALGSHREEPWVD |
| NOV72a | PNSPVLLEDPVLCALADDHKRTPALIALRYQLQRGVVVLAKSYNEQRIRQNVQVFEFQLT |
| NOV72b | PNSPVLLEDPVLCALADDHKRTPALIALRYQLQRGVVVLAKRYNEQRIRQNVQVFEFQLT |
| NOV72c | PNSPVLLEDPVLCALADDHKRTPALIALRYQLQRGVVVLAKSYNEQRIRQNVQVFEFQLT |
| NOV72d | PNSPVLLEDPVLCALADDHKRTPALIALRYQLQRGVVVLAKSYNEQRIRQNVQVFEFQLT |
| NOV72e | PNSPVLLEDPVLCALADDHKRTPALIALRYQLQRGVVVLAKSYNEQRIRQNVQVFEFQLT |
| NOV72f | PNSPVLLEDPVLCALADDHKRTPALIALRYQLQRGVVVLAKSYNEQRIRQNVQVFEFQLT |
| NOV72g | PNSPVLLEDPVLCALADDHKRTPALIALRYQLQRGVVVLAKSYNEQRIRQNVQVFEFQLT |
| NOV72h | PNSPVLLEDPVLCALADDHKRTPALIALRYQLQRGVVVLAKSYNEQRIRQNVQVFEFQLT |
| NOV72i | PNSPVLLEDPVLCALADDHKRTPALIALRYQLQRGVVVLAKSYNEQRIRQNVQVFEFQLT |
| NOV72j | PNSPVLLEDPVLCALADDHKRTPALIALRYQLQRGVVVLAKSYNEQRIRQNVQVFEFQLT |
| NOV72a | SEEMKAIDGLNRNVRYLTLDIFAGPPNYPFSDEY--- |
| NOV72b | SEEMKAIDGLNRNVRYLTLDIFAGPPNYPFSDEY--- |
| NOV72c | SEEMKAIDGLNRNVRYLTLDIFAGPPNYPFSDEY--- |
| NOV72d | SEEMKAIDGLNRNVRYLTLDIFAGPPNYPFSDEY--- |
| NOV72e | SEEMKAIDGLNRNVRYLTLDIFAGPPNYPFSD----- |
| NOV72f | SEEMKAIDGLNRNVRYLTLDIFAGPPNYPFSDEY--- |
| NOV72g | SEEMKAIDGLNRNVRYLTLDIFAGPPNYPFSD----- |
| NOV72h | SEEMKAIDGLNRNVRYLTLDIFAGPPNYPFSDEYLEG |
| NOV72i | SEEMKAIDGLNRNVRYLTLDIFAGPPNYPFSDEY--- |
| NOV72j | SEEMKAIDGLNRNVRYLTLDIFAGPPNYPFSDEY--- |
| NOV72a | (SEQ ID NO: 1048) |
| NOV72b | (SEQ ID NO: 1050) |
| NOV72c | (SEQ ID NO: 1052) |
| NOV72d | (SEQ ID NO: 1054) |
| NOV72e | (SEQ ID NO: 1056) |
| NOV72f | (SEQ ID NO: 1058) |
| NOV72g | (SEQ ID NO: 1060) |
| NOV72h | (SEQ ID NO: 1062) |
| NOV72i | (SEQ ID NO: 1064) |
| NOV72j | (SEQ ID NO: 1066) |

Further analysis of the NOV72a protein yielded the following properties shown in Table 72C.

TABLE 72C
Protein Sequence Properties NOV72a

| | |
|---|---|
| SignalP analysis: | No Known Signal Sequence Indicated |

PSORT II analysis:

| PSG: | a new signal peptide prediction method | | | |
|---|---|---|---|---|
| | N-region: | length 9; | pos.chg 2; | neg.chg 1 |
| | H-region: | length 2; | peak value | −3.30 |
| | PSG score: | −7.70 | | |

| | |
|---|---|
| GvH: | von Heijne's method for signal seq. recognition |
| | GvH score (threshold: −2.1): −5.32 |
| | possible cleavage site: between 27 and 28 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 3.66 (at 253) |
| | ALOM score: 3.66 (number of TMSs: 0) |

MITDISC: discrimination of mitochondrial targeting seq

| | | | |
|---|---|---|---|
| R content: | 0 | Hyd Moment(75): | 0.10 |
| Hyd Moment(95): | 4.27 | G content: | 0 |
| D/E content: | 2 | S/T content: | 1 |
| Score: −7.79 | | | |

Gavel: prediction of cleavage sites for mitochondrial preseq

cleavage site motif not found

NUCDISC: discrimination of nuclear localization signals

pat4: KKHK (3) at 246
pat4: KHKR (3) at 247
pat7: none
bipartite: none
content of basic residues: 13.3%
NLS Score: −0.10

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

```
  34.8%: cytoplasmic
  30.4%: mitochondrial
  30.4%: nuclear
    4.3%: vacuolar
```

>> prediction for CG59693-01 is cyt (k = 23)

A search of the NOV72a protein against the Geneseq database, a proprietary database that contains sequences published in patents and patent publication, yielded several homologous proteins shown in Table 72D.

TABLE 72D
Geneseq Results for NOV72a

| | | NOV72a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB43444 | Human cancer associated protein | 1 . . . 323 | 318/323 (98%) | 0.0 |
| | sequence SEQ ID NO: 889 - Homo | 14 . . . 336 | 318/323 (98%) | |
| | sapiens, 336 aa. [WO200055350- | | | |
| | A1, 21-SEP-2000] | | | |
| AAU85559 | Clone #59314 (L1426P) of lung | 1 . . . 323 | 311/323 (96%) | 0.0 |
| | tumour protein - Homo sapiens, | 1 . . . 323 | 316/323 (97%) | |
| | 323 aa. [WO200204514-A2, 17- | | | |
| | JAN-2002] | | | |
| ABB75050 | Human lung tumour L773P | 29 . . . 323 | 288/295 (97%) | e−168 |
| | recombinant protein sequence | 77 . . . 371 | 290/295 (97%) | |
| | SEQ ID NO: 433 - Homo sapiens, | | | |
| | 371 aa. [WO200200174-A2, 03- | | | |
| | JAN-2002] | | | |
| ABB74958 | Human lung tumour L773P | 29 . . . 323 | 288/295 (97%) | e−168 |
| | protein sequence SEQ ID NO: 172 - | 70 . . . 364 | 290/295 (97%) | |
| | Homo sapiens, 364 aa. | | | |
| | [WO200200174-A2, 03-JAN- | | | |
| | 2002] | | | |
| AAU85520 | L773P lung tumour protein - | 29 . . . 323 | 288/295 (97%) | e−168 |
| | Homo sapiens, 364 aa. | 70 . . . 364 | 290/295 (97%) | |
| | [WO200204514-A2, 17-JAN- | | | |
| | 2002] | | | |

In a BLAST search of public sequence databases, the NOV72a protein was found to have homology to the proteins shown in the BLASTP data in Table 72E.

TABLE 72E
Public BLASTP Results for NOV72a

| | | NOV72a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q04828 | Aldo-keto reductase family 1 | 1 . . . 323 | 323/323 (100%) | 0.0 |
| | member C1 (EC 1.1.1.—) (Trans-1,2- | 1 . . . 323 | 323/323 (100%) | |
| | dihydrobenzene-1,2-diol | | | |
| | dehydrogenase) (EC 1.3.1.20) (High- | | | |
| | affinity hepatic bile acid-binding | | | |
| | protein) (HBAB) (Chlordecone | | | |
| | reductase homolog HAKRC) | | | |
| | (Dihydrodiol dehydrogenase 2) | | | |
| | (DD2) (20 alpha-hydroxysteroid | | | |
| | dehydrogenase) - Homo sapiens | | | |
| | (Human), 323 aa. | | | |
| P52895 | Aldo-keto reductase family 1 | 1 . . . 323 | 316/323 (97%) | 0.0 |
| | member C2 (EC 1.1.1.—) (Trans-1,2- | 1 . . . 323 | 318/323 (97%) | |
| | dihydrobenzene-1,2-diol | | | |
| | dehydrogenase) (EC 1.3.1.20) | | | |
| | (Chlordecone reductase homolog | | | |
| | HAKRD) (Dihydrodiol | | | |
| | dehydrogenase/bile acid-binding | | | |
| | protein) (DD/BABP) (Dihydrodiol | | | |
| | dehydrogenase 2) (DD2) - Homo | | | |
| | sapiens (Human), 323 aa. | | | |
| I73676 | chlordecone reductase homolog | 1 . . . 323 | 313/323 (96%) | 0.0 |
| | (clone HAKRd) - human, 323 aa. | 1 . . . 323 | 317/323 (97%) | |
| I73675 | chlordecone reductase homolog | 4 . . . 323 | 312/320 (97%) | 0.0 |
| | (clone HAKRc) - human, 320 aa | 1 . . . 320 | 313/320 (97%) | |
| | (fragment). | | | |
| Q95JH6 | 3(20)alpha- | 1 . . . 323 | 304/323 (94%) | 0.0 |
| | hydroxysteroid/dihydrodiol/indanol | 1 . . . 323 | 319/323 (98%) | |
| | dehydrogenase (EC 1.1.1.112) - | | | |
| | Macaca fuscata (Japanese macaque), | | | |
| | 323 aa. | | | |

PFam analysis indicates that the NOV72a protein contains the domains shown in the Table 72F.

TABLE 72F
Domain Analysis of NOV72a

| | NOV72a | Identities/Similarities | Expect |
|---|---|---|---|
| Pfam Domain | Match Region | for the Matched Region | Value |
| aldo_ket_red | 10 . . . 303 | 164/369 (44%) | 3.4e−156 |
| | | 274/369 (74%) | |

### Example 73

The NOV73 clone was analyzed, and the nucleotide and encoded polypeptide sequences are shown in Table 73A.

TABLE 73A
NOV73 Sequence Analysis

| | | |
|---|---|---|
| NOV73a, CG59839-02 | SEQ ID NO: 1067 | 4052 bp |

| | | |
|---|---|---|
| DNA Sequence | ORF Start: ATG at 786 | ORF Stop: TAG at 4023 |

CTTCTGCCTCAGCCTCCCGAGTAGCTGGGATGGGACTACAGATGCACGCCACCACACCTGGCAATTTT
TTTTAGTTTTGTGGAGACAAGGTCCCATGTGAGCCACTGTGCCCCACCTTTTAAAGAAATTACCATAA
GGAAGCAACTGTCTTTAGCAAATAGAAGATCATAAACATCCTTCTGGAGATCTTGAGATGTTGACTGC
GGTTTATAGGGCAATGAAAATCTGCCTACCTGAGTGGGCAATGAGACGGGAGTGCCACCTGTGATCCT
TCCAGGGAGCAATTCTTTCTGGTGCCAGGAGCCCAAAGGAAACTCAGTCAACAAGCTACAGATGGGCT
TGCTCCGAGTTTGTTCTAGAAGTCAGAACTGCCAGCTCTGAAAATAGGTCACAAGTGACAGGCCGTCC
CTGGGCTGCTCACAGGAAGTAGCTGCAAAGGTCAGGGAAAGTTTTTAACGACTATACAGGCCAGTGAA
TGACAGGCTTTGAGGAGCTTCTCTTCTTGGCATCTGAAACAGCATACTTTCTGACTACCGCTGTGTGC
TGTGAGGGGCTGAGGATCCAGACGTGGCTGAGGAAGGTCCCGGCTCATGAGGAGGTGGAAGCCTGGGT
GCTTAAACAGATAATCCAGAGTTTGGATGTGAGGTGGTGAATCACTCTGGCTTCATTTGGTTATACTG
GTCAAGAGTTTGGCTCTGGAGCAACGTGTCTGGGTTTGAATCCTGGCTGTGCTTTTGGTGATCCTCCA
TTGTTTTGAAGCACAAGACCTTTAAACCACTGTAGGTATGGACAAGGAAGAAAGGAAGATCATCAATC
AGGGTCAAGAAGATGAAATGGAGATTTATGGTTACAATTTGAGTCGCTGGAAGCTTGCCATAGTTTCT
TTAGGAGTGATTTGCACTGGTGGGTTTCTCCTCCTCCTCCTCTATTGGATGCCTGAGTGGCGGGTGAA
AGCGACCTGTGTCAGAGCTGCAATTAAAGACTGTGACGTAGTGCTGCTGAGGACTACTGATGAATTCA
AAATGTGGTTTTGTGCAAAAATTCGCGTTCTTTCTTTGGAAACTCACCCAATTTCAAGTCCAAAATCT
ATGTCTAATAAGCTTTCAAATGGCCATGCAGTTTGTTTAACTGAGAATCCCACTGGAGAAAATAGGCA
CGGGATCAGTAAATATTCACAGGCTGAATCACAACAGATTCGTTATTTCACCCATCATAGTGTAAAAT
ATTTCTGGAATGATACCATTCACAATTTTGATTTCTTAAAGGGACTGGATGAAGGTGTTTCTTGTACG
TCAATTTATGAAAAGCATAGTGCAGGACTGACAAAGGGGATGCATGCCTACAGAAAACTGCTTTATGG
AGTAAATGAAATTGCTGTAAAAGTGCCTTCTGTTTTTAAGCTTCTAATTAAAGAGGTTCTCAACCCAT
TTTACATTTTCCAGCTGTTCAGTGTTATACTGTGGAGCACTGATGAATACTATTACTATGCTCTAGCT
ATTGTGGTTATGTCCATAGTATCAATCTGTAGCTCACTGTATTCCATTAGAAAGCAATATGTTATGTT
GCATGACATGGTGGCAACTCATAGTACTGTAAGAGTTTCAGTTTGTAGAGTAAATGAAGAAATAGAAG
AAATCTTTTCTACTGACCTTGTGCCAGGAGATGTCATGGTCATTCCATTAAATGGGACAATAATGCCT
TGCGATGCTGTACTTATTAATGGTACCTGCATTGTAAATGAAAGCATGTTAACAGGAGAAAGTGTTCC
AGTGACAAAGACTAACTTGCCAAATCCTTCGGTGGATGTGAAAGGAATAGGAGATGAATTATATAATC
CAGAAACACATAAACGACATACTTTGTTTTGTGGGACAACTGTTATTCAGACTCGTTTCTACACCGGA
GAACTCGTCAAAGCCATAGTTGTTAGAACAGGATTTAGTACTTCCAAAGGACAGCTTGTTCGTTCCAT
ATTGTATCCCAAACCAACTGATTTTAAACTCTACAGAGATGCCTACTTGTTTCTACTATGTCTTGTGG
CAGTTGCTGGCATTGGGTTTATCTACACTATTATTAATAGCATTTTAAATGAGGTACAAGTTGGGGTC
ATAATTATCGAGTCTCTTGATATTATCACAATTACTGTGCCCCCTGCACTTCCTGCTGCAATGACTGC
TGGTATTGTGTATGCTCAGAGAAGACTGAAAAAAATCGGTATTTTCTGTATCAGTCCTCAAAGAATAA
ATATTTGTGGACAGCTCAATCTTGTTTGCTTTGACAAGACTGGAACTCTAACTGAAGATGGTTTAAAT
CTTTGGGGGATTCAACGAGTGGAAAATGCACGGTTTCTTTCACCAGAAGAAAATGTGTGCAATGAGAT
GTTGGTAAAATCCCAGTTTGTTGCTTGTATGGCTACTTGTCATTCACTTACAAAAATTGAAGGAGTGC
TCTCTGGTGATCCACTTGATCTGAAAATGTTTGAGGCTATTGGATGGATTCTGGAAGAAGCAACTGAA
GAAGAAACAGCACTTCATAATCGAATTATGCCCACAGTGGTTCGTCCTCCCAAACAACTGCTTCCTGA
ATCTACCCCTGCAGGAAACCAAGAAATGGCTACTTATGAGATAGGAATTGTTCGCCAGTTCCCATTTT
CTTCTGCTTTGCAACGTATGAGTGTGGTTGCCAGGGTGCTGGGGGATAGGAAAATGGACGCCTACATG
AAAGGAGCGCCCGAGGCCATTGCCGGTCTCTGTAAACCTGAAACAGTTCCTGTCGATTTTCAAAACGT
TTTGGAAGACTTCACTAAACAGGGCTTCCGTGTGATTGCTCTTGCACACAGAAAATTGGAGTCAAAAC
TGACATGGCATAAAGTACAGAATATTAGCAGGGATGCAATTGAGAACAACATGGATTTTATGGGATTA
ATTATAATGCAGAACAAATTAAAGCAAGAAACCCCTGCAGTACTTGAAGATTTGCATAAAGCCAACAT
TCGCACCGTCATGGTCACAGGTGACAGTATGTTGACTGCTGTCTCTGTGGCCAGAGATTGTGGAATGA
TTCTACCTCAGGATAAAGTGATTATTGCTGAAGCATTACCTCCAAAGGATGGGAAAGTTGCCAAAATA
AATTGGCATTATGCAGACTCCCTCACGCAGTGCAGTCATCCATCAGCAATTGACCCAGAGGCTATTCC
GGTTAAATTGGTCCATGATAGCTTAGAGGATCTTCAAATGACTCGTTATCATTTTGCAATGAATGGAA
AATCATTCTCAGTGATACTGGAGCATTTTCAAGACCTTGTTCCTAAGTTGATGTTGCATGGCACCGTG
TTTGCCCGTATGGCACCTGATCAGAAGACACAGTTGATAGAAGCATTGCAAAATGTTGAGTATTTTGT
TGGGATGTGTGGTGATGGCGCAAATGATTGTGGTGCTTTGAAGAGGGCACACGGAGGCATTTCCTTAT
CGGAGCTCGAAGCTTCAGTGGCATCTCCCTTTACCTCTAAGACTCCTAGTATTTCCTGTGTGCCAAAC
CTTATCAGGGAAGGCCGTGCTGCTTTAATAACTTCCTTCTGTGTGTTTAAATTCATGGCATTGTACAG
CATTATCCAGTACTTCAGTGTTACTCTGCTGTATTCTATCTTAAGTAACCTAGGAGACTTCCAGTTTC
TCTTCATTGATCTGGCAATCATTTTGGTAGTGGTATTTACAGTGAGTTTAAATCCTGCCTGGAAAGAA
CTTGTGGCACAAAGACCACCTTCGGGTCTTATATCTGGGGCCCTTCTCTTCTCCGTTTTGTCTCAGAT
TATCATCTGCATTGGATTTCAATCTTTGGGTTTTTTTTGGGTCAAACAGCAACCTTGGTATGAAGTGT
GGCATCCAAAATCAGAGGCTTGTAATACAACAGGAAGCGGGTTTTGGAATTCTTCACACGTAGACAAT
GAAACCGAACTTGATGAAATAATATACAAAATTATGAAAATACCACAGTGTTTTTTATTTCCAGTTTT
CAGTACCTCATAGTGGCAATTGCCTTTTCAAAAGGAAAAC

NOV73a, CG59839-02

| | | | |
|---|---|---|---|
| Protein Sequence | SEQ ID NO: 1068 | 1079 aa | MW at 120773.2kD |

MDKEERKIINQGQEDEMEIYGYNLSRWKLAIVSLGVICTGGFLLLLLYWMPEWRVKATCVRAAIKDCD
VVLLRTTDEFKMWFCAKIRVLSLETHPISSPKSMSNKLSNGHAVCLTENPTGENRHGISKYSQAESQQ
IRYFTHHSVKYFWNDTIHNFDFLKGLDEGVSCTSIYEKHSAGLTKGMHAYRKLLYGVNEIAVKVPSVF
KLLIKEVLNPFYIFQLFSVILWSTDEYYYYALAIVVMSIVSIVSSLYSIRKQYVMLHDMVATHSTVRV
SVCRVNEEIEEIFSTDLVPGDVMVIPLNGTIMPCDAVLINGTCIVNESMLTGESVPVTKTNLPNPSVD
VKGIGDELYNPETHKRHTLFCGTTVIQTRFYTGELVKAIVVRTGFSTSKGQLVRSILYPKPTDFKLYR
DAYLFLLCLVAVAGIGFIYTIINSILNEVQVGVIIIESLDIITITVPPALPAAMTAGIVYAQRRLKKI
GIFCISPQRINICGOLNLVCFDKTGTLTEDGLNLWGIQRVENARFLSPEENVCNEMLVKSQFVACMAT
CHSLTKIEGVLSGDPLDLKMFEAIGWILEEATEEETALHNRIMPTVVRPPKQLLPESTPAGNQEMATY
EIGIVRQFPFSSALQRMSVVARVLGDRKMDAYMKGAPEAIAGLCKPETVPVDFQNVLEDFTKQGFRVI
ALAHRKLESKLTWHKVQNISRDAIENNMDFMGLIIMQNKLKQETPAVLEDLHKANIRTVMVTGDSMLT
AVSVARDCGMILPQDKVIIAEALPPKDGKVAKINWHYADSLTQCSHPSAIDPEAIPVKLVHDSLEDLQ
MTRYHFAMNGKSFSVILEHFQDLVPKLMLHGTVFARMAPDQKTQLIEALQNVEYFVGMCGDGANDCGA
LKRAHGGISLSELEASVASPFTSKTPSISCVPNLIREGRAALITSFCVFKFMALYSIIQYFSVTLLYS
ILSNLGDFQFLFIDLAIILVVVFTVSLNPAWKELVAQRPPSGLISGALLFSVLSQIIICIGFQSLGFF
WVKQQPWYEVWHPKSEACNTTGSGFWNSSHVDNETELDEIIYKIMKIPQCFLFPVFSTS

| | | |
|---|---|---|
| NOV73b, CG59839-01 | SEQ ID NO: 1069 | 2649 bp |

| | | |
|---|---|---|
| DNA Sequence | ORF Start: ATG at 183 | ORF Stop: TAG at 2055 |

CAACTGATTTTAAACTCTACAGAGATGCCTACTTGTTTCTACTATGTCTTGTGGCAGTTGCTGGCATT
GGGTTTATCTACACTATTATTAATAGCATTTTAAATGAGGTACAAGTTGGGGTCATAATTATCGAGTC
TCTTGATATTATCACAATTACTGTGCCCCCTGCACTTCCTGCTGCAATGACTGCTGGTATTGTGTATG
CTCAGAGAAGACTGAAAAAAATCGGTATTTTCTGTATCAGTCCTCAAAGAATAAATATTTGTGGACAG
CTCAATCTTGTTTGCTTTGACAAGACTGGAACTCTAACTGAAGATGGTTTAGATCTTTGGGGGATTCA
ACGAGTGGAAAATGCACGATTTCTTTCACCAGAAGAAAATGTGTGCAATGAGATGTTGGTAAAATCCC
AGTTTGTTGCTTGTATGGCTACTTGTCATTCACTTACAAAAATTGAAGGAGTGCTCTCTGGTGATCCA
CTTGATCTGAAAATGTTTGAGGCTATTGGATGGATTCTGGAAGAAGCAACTGAAGAAGAAACAGCACT
TCATAATCGAATTATGCCCACAGTGGTTCGTCCTCCCAAACAACTGCTTCCTGAATCTACCCCTGCAG
GAAACCAAGAAATGGAGCTGTTTGAACTTCCAGCTACTTATGAGATAGGAATTGTTCGCCAGTTCCCA
TTTTCTTCTGCTTTGCAACGTATGAGTGTGGTTGCCAGGGTGCTGGGGGATAGGAAAATGGACGCCTA
CATGAAAGGAGCGCCCGAGGCCATTGCCGGTCTCTGTAAACCTGAAACAGTTCCTGTCGATTTTCAAA
ACGTTTTGGAAGACTTCACTAAACAGGGCTTCCGTGTGATTGCTCTTGCACACAGAAAATTGGAGTCA
AAACTGACATGGCATAAAGTACAGAATATTAGCAGAGATGCAATTGAGAACAACATGGATTTTATGGG
ATTAATTATAATGCAGAACAAATTAAAGCAAGAAACCCCTGCAGTACTTGAAGATTTGCATAAAGCCA
ACATTCGCACCGTCATGGTCACAGGTGACAGTATGTTGACTGCTGTCTCTGTGGCCAGAGATTGTGGA
ATGATTCTACCTCAGGATAAAGTGATTATTGCTGAAGCATTACCTCCAAAGGATGGGAAAGTTGCCAA
AATAAATTGGCATTATGCAGACTCCCTCACGCAGTGCAGTCATCCATCAGCAATTGACCCAGAGGCTA
TTCCGGTTAAATTGGTCCATGATAGCTTAGAGGATCTTCAAATGACTCGTTATCATTTTGCAATGAAT
GGAAAATCATTCTCAGTGATACTGGAGCATTTTCAAGACCTTGTTCCTAAGTTGATGTTGCATGGCAC
CGTGTTTGCCCGTATGGCACCTGATCAGAAGACACAGTTGATAGAAGCATTGCAAAATGTTGATTATT
TTGTTGGGATGTGTGGTGATGGCGCAAATGATTGTGGTGCTTTGAAGAGGGCACACGGAGGCATTTCC
TTATCGGAGCTCGAAGCTTCAGTGGCATCTCCCTTTACCTCTAAGACTCCTAGTATTTCCTGTGTGCC
AAACCTTATCAGGGAAGGCCGTGCTGCTTTAATAACTTCCTTCTGTGTGTTTAAATTCATGGCATTGT
ACAGCATTATCCAGTACTTCAGTGTTACTCTGCTGTATTCTATCTTAAGTAACCTAGGAGACTTCCAG
TTTCTCTTCATTGATCTGGCAATCATTTTGGTAGTGGTATTTACAATGAGTTTAAATCCTGCCTGGAA
AGAACTTGTGGCACAAAGACCACCTTCGGGTCTTATATCTGGGGCCCTTCTCTTCTCCGTTTTGTCTC
AGATTATCATCTGCATTGGATTTCAATCTTTGGGTTTTTTTTGGGTCAAACAGCAACCTTGGTATGAA
GTGTGGCATCCAAAATCAGATGCTTGTAATACAACAGGAAGCGGGTTTTGGAATTCTTCACACGTAGA
CAATGAAACCGAACTTGATGAACTAATATACAAAATTATGAAAATACCACAGTGTTTTTTATTTCCAG
TTTTCAGTACCTCATAGTGGCAATTGCCTTTTCAAAAGGAAAACCCTTCAGGCAACCTTGCTACAAAA
ATTATTTTTTTGTTTTTTCTGTGATTTTTTTATATATTTTTATATTATTCATCATGTTGTATCCAGTT
GCCTCTGTTGACCAGGTTCTTCAGATAGTGTGTGTACCATATCAGTGGCGTGTAACTATGCTCATCAT
TGTTCTTGTCAATGCCTTTGTGTCTATCACAGTGGAGGAGTCAGTGGATCGGTGGGGAAAATGCTGCT
TACCCTGGGCCCTGGGCTGTAGAAAGAAGACACCAAAGGCAAAGTACATGTATCTGGCGCAGGAGCTC
TTGGTTGATCCAGAATGGCCACCAAAACCTCAGACAACCACAGAAGCTAAAGCTTTAGTTAAGGAGAA
TGGATCATGTCAAATCATCACCATAACATAGCAGTGAATCAGTCTCAGTGGTATTGCTGATAGCAGTA
TTCAGGAATATGTGATTTTAGGAGTTTCTGATCCTGTGTGTCAGAATGGCACTAGTTCAGTTTATGTC
CCTTCTGATATAGTAGCTTATTTGACAGCTTTGCTCTTCCTTAAAATAAAAAAAAAAAAAAAAAA

NOV73b, CG59839-01

| | | | |
|---|---|---|---|
| Protein Sequence | SEQ ID NO: 1070 | 624 aa | MW at 69590.1kD |

MTAGIVYAQRRLKKIGIFCISPQRINICGQLNLVCFDKTGTLTEDGLDLWGIQRVENARFLSPEENVC
NEMLVKSQFVACMATCHSLTKIEGVLSGDPLDLKMFEAIGWILEEATEEETALHNRIMPTVVRPPKQL
LPESTPAGNQEMELFELPATYEIGIVRQFPFSSALQRMSVVARVLGDRKMDAYMKGAPEAIAGLCKPE
TVPVDFQNVLEDFTKQGFRVIALAHRKLESKLTWHKVQNISRDAIENNMDFMGLIIMQNKLKQETPAV
LEDLHKANIRTVMVTGDSMLTAVSVARDCGMILPQDKVIIAEALPPKDGKVAKINWHYADSLTQCSHP
SAIDPEAIPVKLVHDSLEDLQMTRYHFAMNGKSFSVILEHFQDLVPKLMLHGTVFARMAPDQKTGLIE
ALQNVDYFVGMCGDGANDCGALKRAHGGISLSELEASVASPFTSKTPSISCVPNLIREGRAALITSFC
VFKFMALYSIIQYFSVTLLYSILSNLGDFQFLFIDLAIILVVVFTMSLNPAWKELVAQRPPSGLISGA
LLFSVLSQIIICIGFQSLGFFWVKQQPWYEVWHPKSDACNTTGSGFWNSSHVDNETELDELIYKIMKI
PQCFLFPVFSTS

A ClustalW comparison of the above protein sequences yields the following sequence alignment shown in Table 73B.

TABLE 73B
Comparison of the NOV73 protein sequences.

| | |
|---|---|
| NOV73a | MDKEERKIINQGQEDEMEIYGYNLSRWKLAIVSLGVICTGGFLLLLLYWMPEWRVKATCV |
| NOV73b | ------------------------------------------------------------ |
| NOV73a | RAAIKDCDVVLLRTTDEFKMWFCAKIRVLSLETHPISSPKSMSNKLSNGHAVCLTENPTG |
| NOV73b | ------------------------------------------------------------ |
| NOV73a | ENRHGISKYSQAESQQIRYFTHHSVKYFWNDTIHNFDFLKGLDEGVSCTSIYEKHSAGLT |
| NOV73b | ------------------------------------------------------------ |
| NOV73a | KGMHAYRKLLYGVNEIAVKVPSVFKLLIKEVLNPFYIFQLFSVILWSTDEYYYYALAIVV |
| NOV73b | ------------------------------------------------------------ |
| NOV73a | MSIVSIVSSLYSIRKQYVMLHDMVATHSTVRVSVCRVNEEIEEIFSTDLVPGDVMVIPLN |
| NOV73b | ------------------------------------------------------------ |
| NOV73a | GTIMPCDAVLINGTCIVNESMLTGESVPVTKTNLPNPSVDVKGIGDELYNPETHKRHTLF |
| NOV73b | ------------------------------------------------------------ |
| NOV73a | CGTTVIQTRFYTGELVKAIVVRTGFSTSKGQLVRSILYPKPTDFKLYRDAYLFLLCLVAV |
| NOV73b | ------------------------------------------------------------ |
| NOV73a | AGIGFIYTIINSILNEVQVGVIIIESLDIITITVPPALPAAMTAGIVYAQRRLKKIGIFC |
| NOV73b | -----------------------------------------MTAGIVYAQRRLKKIGIFC |
| NOV73a | ISPQRINICGQLNLVCFDKTGTLTEDGLNLWGIQRVENARFLSPEENVCNEMLVKSQFVA |
| NOV73b | ISPQRINICGQLNLVCFDKTGTLTEDGLDLWGIQRVENARFLSPEENVCNEMLVKSQFVA |
| NOV73a | CMATCHSLTKIEGVLSGDPLDLKMFEAIGWILEEATEEETALHNRIMPTVVRPPKQLLPE |
| NOV73b | CMATCHSLTKIEGVLSGDPLDLKMFEAIGWILEEATEEETALHNRIMPTVVRPPKQLLPE |
| NOV73a | STPAGNQ------EMATYEIGIVRQFPFSSALQRMSVVARVLGDRKMDAYMKGAPEAIAG |
| NOV73b | STPAGNQEMELFELPATYEIGIVRQFPFSSALQRMSVVARVLGDRKMDAYMKGAPEAIAG |
| NOV73a | LCKPETVPVDFQNVLEDFTKQGFRVIALAHRKLESKLTWHKVQNISRDAIENNMDFMGLI |
| NOV73b | LCKPETVPVDFQNVLEDFTKQGFRVIALAHRKLESKLTWHKVQNISRDAIENNMDFMGLI |
| NOV73a | IMQNKLKQETPAVLEDLHKANIRTVMVTGDSMLTAVSVARDCGMILPQDKVIIAEALPPK |
| NOV73b | IMQNKLKQETPAVLEDLHKANIRTVMVTGDSMLTAVSVARDCGMILPQDKVIIAEALPPK |
| NOV73a | DGKVAKINWHYADSLTQCSHPSAIDPEAIPVKLVHDSLEDLQMTRYHFANNGKSFSVILE |
| NOV73b | DGKVAKINWHYADSLTQCSHPSAIDPEAIPVKLVHDSLEDLQMTRYHFANNGKSFSVILE |
| NOV73a | HFQDLVPKLMLHGTVFARMAPDQKTQLIEALQNVDYFVGMCGDGANDCGALKRAHGGISL |
| NOV73b | HFQDLVPKLMLHGTVFARMAPDQKTQLIEALQNVDYFVGMCGDGANDCGALKRAHGGISL |
| NOV73a | SELEASVASPFTSKTPSISCVPNLIREGRAALITSFCVFKFMALYSIIQYFSVTLLYSIL |
| NOV73b | SELEASVASPFTSKTPSISCVPNLIREGRAALITSFCVFKFMALYSIIQYFSVTLLYSIL |
| NOV73a | SNLGDFQFLFIDLAIILVVVFTVSLNPAWKELVAQRPPSGLISGALLFSVLSQIIICIGF |
| NOV73b | SNLGDFQFLFIDLAIILVVVFTMSLNPAWKELVAQRPPSGLISGALLFSVLSQIIICIGF |
| NOV73a | QSLGFFWVKQQPWYEVWHPKSEACNTTGSGFWNSSHVDNETELDEIIYKIMKIPQCFLFP |
| NOV73b | QSLGFFWVKQQPWYEVWHPKSDACNTTGSGFNNSSHVDNETELDELIYKIMKIPQCFLFP |
| NOV73a | VFSTS |
| NOV73b | VFSTS |
| NOV73a | (SEQ ID NO: 1068) |
| NOV73b | (SEQ ID NO: 1070) |

Further analysis of the NOV73a protein yielded the following properties shown in Table 73C.

TABLE 73C
Protein Sequence Properties NOV73a

| | |
|---|---|
| SignalP analysis: | Cleavage site between residues 58 and 59 |

PSORT II analysis:

| PSG: | a new signal peptide prediction method | | | |
|---|---|---|---|---|
| | N-region: | length 7; | pos.chg 3; | neg.chg 3 |
| | H-region: | length 6; | peak value | −12.14 |
| | PSG score: | −16.54 | | |

| | |
|---|---|
| GvH: | von Heijne's method for signal seq. recognition |
| | GvH score (threshold: −2.1): −4.30 |
| | possible cleavage site: between 41 and 42 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 8 | |
| | INTEGRAL | Likelihood = −8.44 Transmembrane 31-47 |
| | INTEGRAL | Likelihood = −6.37 Transmembrane 234-250 |
| | INTEGRAL | Likelihood = 0.32 Transmembrane 295-311 |
| | INTEGRAL | Likelihood = −9.08 Transmembrane 410-426 |
| | INTEGRAL | Likelihood = −2.81 Transmembrane 439-455 |
| | INTEGRAL | Likelihood = −2.92 Transmembrane 926-942 |
| | INTEGRAL | Likelihood =−11.15 Transmembrane 963-979 |
| | INTEGRAL | Likelihood = −6.37 Transmembrane 996-1012 |
| | PERIPHERAL | Likelihood = 1.11 (at 211) |
| | ALOM score: | −11.15 (number of TMSs: 8) |

| | |
|---|---|
| MTOP: | Prediction of membrane topology (Hartmann et al.) |
| | Center position for calculation: 38 |
| | Charge difference: 4.0 C(2.0) − N(−2.0) |
| | C > N: C-terminal side will be inside |

>>> membrane topology: type 3b
MITDISC: discrimination of mitochondrial targeting seq

| | | | |
|---|---|---|---|
| R content: | 0 | Hyd Moment(75): | 8.50 |
| Hyd Moment(95): | 5.62 | G content: | 0 |
| D/E content: | 2 | S/T content: | 0 |
| Score: −6.77 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 9.1%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

```
  55.6%: endoplasmic reticulum
  11.1%: mitochondrial
  11.1%: vacuolar
  11.1%: vesicles of secretory system
  11.1%: Golgi
```

>> prediction for CG59839-02 is end (k = 9)

A search of the NOV73a protein against the Geneseq database, a proprietary database that contains sequences published in patents and patent publication, yielded several homologous proteins shown in Table 73D.

TABLE 73D
Geneseq Results for NOV73a

| | | NOV73a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU91185 | Human HEAT-2 polypeptide - | 1 . . . 1059 | 1044/1065 (98%) | 0.0 |
| | Homo sapiens, 1256 aa. | 1 . . . 1065 | 1053/1065 (98%) | |
| | [WO200216591-A2, 28-FEB- | | | |
| | 2002] | | | |
| AAO14197 | Human transporter and ion | 1 . . . 1059 | 1039/1065 (97%) | 0.0 |
| | channel TRICH-14 - Homo | 1 . . . 1065 | 1049/1065 (97%) | |
| | sapiens, 1256 aa. | | | |
| | [WO200204520-A2, 17-JAN- | | | |
| | 2002] | | | |
| ABU11063 | Human protein NOV27 - Homo | 183 . . . 1059 | 784/883 (88%) | 0.0 |
| | sapiens, 973 aa. | 1 . . . 812 | 792/883 (88%) | |
| | [WO200281629-A2, 17-OCT- | | | |
| | 2002] | | | |
| ABP69451 | Human polypeptide SEQ ID NO | 462 . . . 1059 | 593/604 (98%) | 0.0 |
| | 1498 - Homo sapiens, 765 aa. | 1 . . . 604 | 598/604 (98%) | |
| | [WO200270539-A2, 12-SEP- | | | |
| | 2002] | | | |
| AAB40996 | Human ORFX ORF760 | 509 . . . 1059 | 547/557 (98%) | 0.0 |
| | polypeptide sequence SEQ ID | 2 . . . 558 | 551/557 (98%) | |
| | NO: 1520 - Homo sapiens, 692 | | | |
| | aa. [WO200058473-A2, 05- | | | |
| | OCT-2000] | | | |

In a BLAST search of public sequence databases, the NOV73a protein was found to have homology to the proteins shown in the BLASTP data in Table 73E.

TABLE 73E
Public BLASTP Results for NOV73a

| | | NOV73a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9H7F0 | Probable cation- | 1 . . . 1059 | 1045/1065 (98%) | 0.0 |
| | transporting ATPase 3 (EC | 1 . . . 1065 | 1054/1065 (98%) | |
| | 3.6.3.—) (ATPase family | | | |
| | homolog up-regulated in | | | |
| | senescence cells 1) - Homo | | | |
| | sapiens (Human), 1130 aa. | | | |
| CAD29017 | Sequence 5 from Patent | 1 . . . 1059 | 1044/1065 (98%) | 0.0 |
| | WO0216591 - Homo | 1 . . . 1065 | 1053/1065 (98%) | |
| | sapiens (Human), 1256 aa. | | | |
| Q96KS1 | Hypothetical protein - | 280 . . . 954 | 655/681 (96%) | 0.0 |
| | Homo sapiens (Human), | 3 . . . 680 | 662/681 (97%) | |
| | 701 aa. | | | |
| Q95JN5 | Hypothetical 56.9 kDa | 1 . . . 492 | 492/492 (100%) | 0.0 |
| | protein - Macaca | 1 . . . 492 | 492/492 (100%) | |
| | fascicularis (Crab eating | | | |
| | macaque) (Cynomolgus | | | |
| | monkey), 504 aa | | | |
| | (fragment). | | | |
| Q95JN5 | Probable cation- | 1 . . . 492 | 492/492 (100%) | 0.0 |
| | transporting ATPase 3 (EC | 1 . . . 492 | 492/492 (100%) | |
| | 3.6.3.—) (ATPase family | | | |
| | homolog up-regulated in | | | |
| | senescence cells 1) - | | | |
| | Macaca fascicularis (Crab | | | |
| | eating macaque) | | | |
| | (Cynomolgus monkey), | | | |
| | 492 aa (fragment). | | | |

PFam analysis indicates that the NOV73a protein contains the domains shown in the Table 73F.

TABLE 73F
Domain Analysis of NOV73a

| | | Identities/ | |
|---|---|---|---|
| | NOV73a | Similarities | Expect |
| Pfam Domain | Match Region | for the Matched Region | Value |
| Cation_ATPase_N | 160 . . . 227 | 15/87 (17%) | 0.041 |
| | | 41/87 (47%) | |
| E1-E2_ATPase | 239 . . . 488 | 58/267 (22%) | 4.3e−07 |
| | | 162/267 (61%) | |
| Hydrolase | 492 . . . 898 | 40/417 (10%) | 0.0073 |
| | | 243/417 (58%) | |

### Example 74

The NOV74 clone was analyzed, and the nucleotide and encoded polypeptide sequences are shown in Table 74A.

TABLE 74A
NOV74 Sequence Analysis

| | | |
|---|---|---|
| NOV74a, CG90866-04 | SEQ ID NO: 1071 | 2937 bp |

| | | |
|---|---|---|
| DNA Sequence | ORF Start: ATG at 1 | ORF Stop: TAA at 2935 |

ATGCAGCCCCTGGACTTTAGTTCAGGGGGAAGTGACCCCAACATCAGCCTCTCAGAAAAGATCCGAGA
TCAGCTTGTTGTTGGACAGCTGATTCCAGACTGCTATGTAGAACTTGAAAAAATCATTTTATCGGAGC
GTAAAAATGTGCCAATTGAATTTCCCGTAATTGACCGGAAACGATTATTACAACTAGTGAGAGAAAAT
CAGCTGCAGTTAGATGAAAATGAGCTTCCTCACGCAGTTCACTTTCTAAATGAATCAGGAGTCCTTCT
TCATTTTCAAGACCCAGCACTGCAGTTAAGTGACTTGTACTTTGTGGAACCCAAGTGGCTTTGTAAAA
TCATGGCACAGGATGTTAGCAGCATTTTTGGCCTTTATATTCGAGACATTTTGACAGTGAAAGTGGAA
GGTTGTCCAAAACACCCTAAGGGCATTATTTCGCGTAGAGATGTGGAAAAATTTCTTTCAAAAAAAAG
GAAATTTCCAAAGAACTACATGTCACAGTATTTTAAGCTCCTAGAAAAATTCCAGATTGCTTTGCCAA
TAGGAGAAGAATATTTGCTGGTTCCAAGCATTTTGTCTGACCACAGGCCTGTGATAGAGCTTCCCCAT
TGTGAGAACTCTGAAATTATCATCCGACTATATGAAATGCCTTATTTTCCAATGGGATTTTGGTCAAG
ATTAATCAATCGATTACTTGAGATTTCACCTTACATGCTTTCAGGGAGAGGCTGTATTCTTTTGGGCC
AAGTTGTGGACCACATTGATTCTCTCATGGAAGAATGGTTTCCTGGGTTGCTGGAGATTGATATTTGT
GGTGAAGGAGAAACTCTGTTGAAGAAATGGGCATTATATAGTTTTAATGATGGTGAAGAACATCAAAA
AATCTTACTTGATGACTTGATGAAGAAAGCAGAGGAAGGAGATCTCTTAGTAAATCCAGATCAACCAA
GGCTCACCATTCCAATATCTCAGATTGCCCCTGACTTGATTTTGGCTGACCCGCCTAGAAATATTATG
TTGAATAATGATGAGTTGGAATTTGAACAAGCTCCAGAGTTTCTCCTAGACTGTTTTGTGTGTATTCA
CTTATATCCATCAAGTGACTACATTTCAAGGCACTATATGAGAACCATAAATATTGTACAAACAGGAT
TTGCTAAATGTCGGTGGAGAGTAACAGTCCACGGGGCTGATCATGGTGATGGCAGTTTTGGATCAGTT
TACCGAGCAGCCTATGAAGGAGAAGAAGTGGCTGTGAAGATTTTTAATAAACATACATCACTCAGGCT
GTTAAGACAAGAGCTTGTGGTGCTTTGCCACCTCCACCACCCCAGTTTGATATCTTTGCTGGCAGCTG
GGATTCGTCCCCGGATGTTGGTGATGGAGTTAGCCTCCAAGGGTTCCTTGGATCGCCTGCTTCAGCAG
GACAAAGCCAGCCTCACTAGAACCCTACAGCACAGGATTGCACTCCACGTAGCTGATGGTTTGAGATA
CCTCCACTCAGCCATGATTATATACCGAGACCTGAAACCCCACAATGTGCTGCTTTTCACACTGTATC
CCAATGCTGCCATCATTGCAAAGATTGCTGACTACGGCATTGCTCAGTACTGCTGTAGAATGGGGATA
AAAACATCAGAGGGCACACCAGGTTTTCGTGCACCTGAAGTTGCCAGAGGAAATGTCATTTATAACCA
ACAGGCTGATGTTTATTCATTTGGTTTACTACTCTATGACATTTTGACAACTGGAGGTAGAATAGTAG
AGGGTTTGAAGTTTCCAAATGAGTTTGATGAATTAGAAATACAAGGAAAATTACCTGATCCAGTTAAA
GAATATGGTTGTGCCCCATGGCCTATGGTTGAGAAATTAATTAAACAGTGTTTGAAAGAAAATCCTCA
AGAAAGGCCTACTTCTGCCCAGGTATTCGACATTTTGAATTCAGCTGAATTAGTCTGTCTGACGAGAC
GCATTTTATTACCTAAAAACGTAATTGTTGAATGCATGGTTGCTACACATCACAACAGCAGGAATGCA
AGCATTTGGCTGGGCTGTGGGCACACCGACAGAGGACAGCTCTCATTTCTTGACTTAAATACTGAAGG
ATACACTTCTGAGAGCAAACAAAAAAATTTTCTTTTGGTTGGAACCGCTGATGGCAAGTTAGCAATTT
TTGAAGATAAGACTGTTAAGCTTAAAGGAGCTGCTCCTTTGAAGATACTAAATATAGGAAATGTCAGT
ACTCCATTGATGTGTTTGAGTGAATCCACAAATTCAACGGAAAGAAATGTAATGTGGGGAGGATGTGG
CACAAAGATTTTCTCCTTTTCTAATGATTTCACCATTCAGAAACTCATTGAGACAAGAACAAGCCAAC
TGTTTTCTTATGCAGCTTTCAGTGATTCCAACATCATAACAGTGGTGGTAGACACTGCTCTCTATATT
GCTAAGCAAAATAGCCCTGTTGTGGAAGTGTGGGATAAGAAAACTGAAAAACTCTGTGGACTAATAGA
CTGCGTGCACTTTTTAAGGGAGGTAATGGTAAAAGAAAACAAGGAATCAAAACACAAAATGTCTTATT
CTGGGAGAGTGAAAACCCTCTGCCTTCAGAAGAACACTGCTCTTTGGATAGGAACTGGAGGAGGCCAT
ATTTTACTCCTGGATCTTTCAACTCGTCGACTTATACGTGTAATTTACAACTTTTGTAATTCGGTCAG
AGTCATGATGACAGCACAGCTAGGCAGCCTTAAAAATGTCATGCTGGTATTGGGCTACAACCGGAAAA
ATACTGAAGGTACACAAAAGCAGAAGAGATACAATCTTGCTTGACCGTTTGGGACATCCAATCTTCCA
CATGAAGTGCAAAATTTAGAAAAACACATTGAAGTGAGAAAAGAATTAGCTGAAAAAATGAGACGAAC
ATCTGTTGAGTAA

NOV74a, CG90866-04

| | | | |
|---|---|---|---|
| Protein Sequence | SEQ ID NO: 1072 | 978 aa | MW at 111173.8kD |

MQPLDFSSGGSDPNISLSEKIRDQLVVGQLIPDCYVELEKIILSERKNVPIEFPVIDRKRLLQLVREN
QLQLDENELPHAVHFLNESGVLLHFQDPALQLSDLYFVEPKWLCKIMAQDVSSIFGLYIRDILTVKVE
GCPKHPKGIISRRDVEKFLSKKRKFPKNYMSQYFKLLEKFQIALPIGEEYLLVPSSLSDHRPVIELPH
CENSEIIIRLYEMPYFPMGFWSRLINRLLEISPYMLSGRGCILLGQVVDHIDSLMEEWFPGLLEIDIC
GEGETLLKKWALYSFNDGEEHQKILLDDLMKKAEEGDLLVNPDQPRLTIPISQIAPDLILADPPRNIM
LNNDELEFEQAPEFLLDCFVCIHLYPSSDYISRHYMRTINIVQTGFAKCRWRVTVHGADHGDGSFGSV
YRAAYEGEEVAVKIFNKHTSLRLLRQELVVLCHLHHPSLISLLAAGIRPRNLVMELASKGSLDRLLQQ
DKASLTRTLQHRIALHVADGLRYLHSAMIIYRDLKPHNVLLFTLYPNAAIIAKIADYGIAQYCCRMGI
KTSEGTPGFRAPEVARGNVIYNQQADVYSFGLLLYDILTTGGRIVEGLKFPNEFDELEIQGKLPDPVK
EYGCAPWPMVEKLIKQCLKENPQERPTSAQVFDILNSAELVCLTRRILLPKNVIVECMVATHHNSRNA
SIWLGCGHTDRGQLSFLDLNTEGYTSESKQKNFLLVGTADGKIAIFEDKTVKLKGAAPLKILNIGNVS
TPLMCLSESTNSTERNVMWGGCGTKIFSFSNDFTIQKLIETRTSQLFSYAAFSDSNIITVVVDTALYI
AKQNSPVVEVWDKKTEKLCGLIDCVHFLREVMVKENKESKHKMSYSGRVKTLCLQKNTALWIGTGGGH
ILLLDLSTRRLIRVIYNFCNSVRVMMTAQLGSLKNVMLVLGYNRKNTEGTQKQKEIQSCLTVWDINLP
HEVQNLEKHIEVRKELAEKMRRTSVE

| | | |
|---|---|---|
| NOV74b, CG90866-03 | SEQ ID NO: 1073 | 2687 bp |

| | | |
|---|---|---|
| DNA Sequence | ORF Start: ATG at 108 | ORF Stop: TAA at 2658 |

ACGCAGTTCACTTTCTAAATGAATCAGGAGTCCTTCTTCATTTTCAAGACCCAGCACTGCAGTTAAGT
GACTTGTACTTTGTGGAACCCAAGTGGCTTTGTAAAATCATGGCACAGATTTTGACAGTGAAAGTGGA
AGGTTGTCCAAAACACCCTAAGGGCATTATTTCGCGTAGAGATGTGGAAAAATTTCTTTCAAAAAAAA
GGAAATTTCCAAAGAACTACATGTCACAGTATTTTAAGCTCCTAGAAAAATTCCAGATTGCTTTGCCA
ATAGGAGAAGAATATTTGCTGGTTCCAAGCAGTTTGTCTGACCACAGGCCTGTGATAGAGCTTCCCCA
TTGTGAGAACTCTGAAATTATCATCCGACTATATGAAATGCCTTATTTTCCAATGGGATTTTGGTCAA
GATTAATCAATCGATTACTTGAGATTTCACCTTACATGCTTTCAGGGAGAGAACGAGCACTTCGCCCA
AACAGAATGTATTGGCGACAAGGCATTTACTTAAATTGGTCTCCTGAAGCTTATTGTCTGGTAGGATC
TGAAGTCTTAGACAATCATCCAGAGAGTTTCTTAAAAATTACAGTTCTTCTTCTTGTAGAAAACTGTA
TTCTTTTGGGCCAAGTTGTGGACCACATTGATTCTCTCATGGAAGAATGGTTTCCTGGGTTGCTGGAG
ATTGATATTTGTGGTGAAGGAGAAACTCTGTTGAAGAAATGGGCATTATATAGTTTTAATGATGGCGA
AGAACATCAAAAAATCTTACTTGATGACTTGATGAAGAAAGCAGAGGAAGGAGATCTCTTAGTAAATC
CAGATCAACCAAGGCTCACCATTCCAATATCTCAGATTGCCCCTGACTTGATTTTGGCTGACCTGCCT
AGAAATATTATGTTGAATAATGATGAGTTGGAATTTGAACAAGCTCCAGAGTTTCTCCTAGGTGATGG
CAGTTTTGGATCAGTTTACCGAGCAGCCTATGAAGGAGAAGAAGTGGCTGTGAAGATTTTTAATAAAC
ATACATCACTCAGGCTGTTAAGACAAGAGCTTGTGGTGCTTTGCCACCTCCACCACCCCAGTTTGATA
TCTTTGCTGGCAGCTGGGATTCGTCCCCGGATGTTGGTGATGGAGTTAGCCTCCAAGGGTTCCTTGGA
TCGCCTGCTTCAGCAGGACAAAGCCAGCCTCACTAGAACCCTACAGCACAGGATTGCACTCCACGTAG
CTGATGGTTTGAGATACCTCCACTCAGCCATGATTATATACCGAGACCTGAAACCCCACAATGTGCTG
CTTTTCACACTGTATCCCAATGCTGCCATCATTGCAAAGATTGCTGACTACGGCATTGCTCAGTACTG
CTGTAGAATGGGGATAAAAACATCAGAGGGCACACCAGGTTTTCGTGCACCTGAAGTTGCCAGAGGAA
ATGTCATTTATAACCAACAGGCTGATGTTTATTCATTTGGTTTACTACTCTATGACATTTTGACAACT
GGAGGTAGAATAGTAGAGGGTTTGAAGTTTCCAAATGAGTTTGATGAATTAGAAATACAAGGAAAATT
ACCTGATCCAGTTAAAGAATATGGTTGTGCCCCATGGCCTATGGTTGAGAAATTAATTAAACAGTGTT
TGAAAGAAAATCCTCAAGAAAGGCCTACTTCTGCCCAGGTATTCGACATTTTGAATTCAGCTGAATTA
GTCTGTCTGACGAGACGCATTTTATTACCTAAAAACGTAATTGTTGAATGCATGGTTGCTACACATCA
CAACAGCAGGAATGCAAGCATTTGGCTGGGCTGTGGGCACACCGACAGAGGACAGCTCTCATTTCTTG
ACTTAAATACTGAAGGATACACTTCTGAGGAAGTTGCTGATAGTAGAATATTGTGCTTAGCCTTGGTG
CATCTTCCTGTTGAAAAGGAAAGCTGGATTGTGTCTGGGACACAGTCTGGTACTCTCCTGGTCATCAA
TACCGAAGATGGGAAAAAGAGACATACCCTAGAAAAGATGACTGATTCTGTCACTTGTTTGTATTGCA
ATTCCTTTTCCAAGCAAAGCAAACAAAAAAATTTTCTTTTGGTTGGAACCGCTGATGGCAAGTTAGCA
ATTTTTGAAGATAAGACTGTTAAGCTTAAAGGAGCTGCTCCTTTGAAGATACTAAATATAGGAAATGT
CAGTACTCCATTGATGTGTTTGAGTGAATCCACAAATTCAACGGAAAGAAATGTAATGTGGGGAGGAT
GTGGCACAAAGATTTTCTCCTTTTCTAATGATTTCACCATTCAGAAACTCATTGAGACAAGAACAAGC
CAACTGTTTTCTTATGCAGCTTTCAGTGATTCCAACATCATAACAGTGGTGGTAGACACTGCTCTCTA
TATTGCTAAGCAAAATAGCCCTGTTGTGGAAGTGTGGGATAAGAAAACTGAAAAACTCTGTGGACTAA
TAGACTGCGTGCACTTTTTAAGCCTTAAAAATGTCATGCTGGTATTGGGCTACAACCGGAAAAATACT
GAAGGTACACAAAAGCAGAAAGAGATACAATCTTGCTTGACCGTTTGGGACATCAATCTTCCACATGA
AGTGCAAAATTTAGAAAAACACATTGAAGTGAGAAAAGAATTAGCTGAAAAAATGAGACGAACATCTG
TTGAGTAAGAGAGAAATAGGAATTGTCTTTGGATA

NOV74b, CG90866-03

| | | | |
|---|---|---|---|
| Protein Sequence | SEQ ID NO: 1074 | 850 aa | MW at 96332.5kD |

MAQILTVKVEGCPKHPKGIISRRDVEKFLSKKRKFPKNYMSQYFKLLEKFQIALPIGEEYLLVPSSLS
DHRPVIELPHCENSEIIIRLYEMPYFPMGFWSRLINRLLEISPYMLSGRERALRPNRMYWRQGIYLNW
SPEAYCLVGSEVLDNHPESFLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDLILADLPRNIMLNNDELEFE
QAPEFLLGDGSFGSVYRAAYEGEEVAVKIFNKHTSLRLLRQELVVLCHLHHPSLISLLAAGIRPRMLV
MELASKGSLDRLLQQDKASLTRTLQHRIALHVALGLRYLHSAMIIYRDLKPHNVLLFTLYPNAAIIAK
IADYGIAQYCCRMGIKTSEGTPGFRAPEVARGNVIYNQQADVYSFGLLLYDILTTGGRIVEGLKFPNE
FDELEIQGKLPDPVKEYGCAPWPMVEKLIKQCLKENPQERPTSAQVFDILNSAELVCLTRRILLPKNV
IVECMVATHHNSRNASIWLGCGHTDRGQLSFLDLNTEGYTSEEVADSRILCLALVHLPVEKESWIVSG
TQSGTLLVINTEDGKKRHTLEKMTDSVTCLYCNSFSKQSKQKNFLLVGTADGKLAIFEDKTVKLKGAA
PLKILNIGNVSTPLMCLSESTNSTERNVMWGGCGTKIFSFSNDFTIQKLIETRTSQLFSYAAFSDSNI
ITVVVDTALYIAKQNSPVVEVWDKKTEKICGLIDCVHFLSLKNVMLVLGYNRKNTEGTQKQKEIQSCL
TVWDINLPHEVQNLEKMIEVRKELAEKMRRTSVE

| | | |
|---|---|---|
| NOV74c, CG90866-01 | SEQ ID NO: 1075 | 3052 bp |

| | | |
|---|---|---|
| DNA Sequence | ORF Start: ATG at 108 | ORF Stop: TAA at 2853 |

ACGCAGTTCACTTTCTAAATGAATCAGGAGTCCTTCTTCATTTTCAAGACCCAGCACTGCAGTTAAGT
GACTTGTACTTTGTGGAACCCAAGTGGCTTTGTAAAATCATGGCACAGATTTTGACAGTGAAAGTGGA
AGGTTGTCCAAAACACCCTAAGGGCATTATTTCGCGTAGAGATGTGGAAAAATTTCTTTCAAAAAAAA
GGAAATTTCCAAAGAACTACATGTCACAGTATTTTAAGCTCCTAGAAAAATTCCAGATTGCTTTGCCA
ATAGGAGAAGAATATTTGCTGGTTCCAAGCAGTTTGTCTGACCACAGGCCTGTGATAGAGCTTCCCCA
TTGTGAGAACTCTGAAATTATCATCCGACTATATGAAATGCCTTATTTTCCAATGGGATTTTGGTCAA
GATTAATCAATCGATTACTTGAGATTTCACCTTACATGCTTTCAGGGAGAGAACGAGCACTTCGCCCA
AACAGAATGTATTGGCGACAAGGCATTTACTTAAATTGGTCTCCTGAAGCTTATTGTCTGGTAGGATC
TGAAGTCTTAGACAATCATCCAGAGAGTTTCTTAAAAATTACAGTTCTTCTTCTTGTAGAAAACTGTA
TTCTTTTGGGCCAAGTTGTGGACCACATTGATTCTCTCATGGAAGAATGGTTTCCTGGGTTGCTGGAG
ATTGATATTTGTGGTGAAGGAGAAACTCTGTTGAAGAAATGGGCATTATATAGTTTTAATGATGGCGA
AGAACATCAAAAAATCTTACTTGATGACTTGATGAAGAAAGCAGAGGAAGGAGATCTCTTAGTAAATC
CAGATCAACCAAGGCTCACCATTCCAATATCTCAGATTGCCCCTGACTTGATTTTGGCTGACCTGCCT
AGAAATATTATGTTGAATAATGATGAGTTGGAATTTGAACAAGCTCCAGAGTTTCTCCTAGGTGATGG
CAGTTTTGGATCAGTTTACCGAGCAGCCTATGAAGGAGAAGAAGTGGCTGTGAAGATTTTTAATAAAC
ATACATCACTCAGGCTGTTAAGACAAGAGCTTGTGGTGCTTTGCCACCTCCACCACCCCAGTTTGATA
TCTTTGCTGGCAGCTGGGATTCGTCCCCGGATGTTGGTGATGGAGTTAGCCTCCAAGGGTTCCTTGGA
TCGCCTGCTTCAGCAGGACAAAGCCAGCCTCACTAGAACCCTACAGCACAGGATTGCACTCCACGTAG
CTGATGGTTTGAGATACCTCCACTCAGCCATGATTATATACCGAGACCTGAAACCCCACAATGTGCTG
CTTTTCACACTGTATCCCAATGCTGCCATCATTGCAAAGATTGCTGACTACGGCATTGCTCAGTACTG
CTGTAGAATGGGGATAAAAACATCAGAGGGCACACCAGGTTTTCGTGCACCTGAAGTTGCCAGAGGAA
ATGTCATTTATAACCAACAGGCTGATGTTTATTCATTTGGTTTACTACTCTATGACATTTTGACAACT
GGAGGTAGAATAGTAGAGGGTTTGAAGTTTCCAAATGAGTTTGATGAATTAGAAATACAAGGAAAATT
ACCTGATCCAGTTAAAGAATATGGTTGTGCCCCATGGCCTATGGTTGAGAAATTAATTAAACAGTGTT
TGAAAGAAAATCCTCAAGAAAGGCCTACTTCTGCCCAGGTATTCGACATTTTGAATTCAGCTGAATTA
TCAGCTGAATTAGTCTGTCTGACGAGACGCATTTTATTACCTAAAAACGTAATTGTTGAATGCATGGT
TGCTACACATCACAACAGCAGGAATGCAAGCATTTGGCTGGGCTGTGGGCACACCGACAGAGGACAGC
TCTCATTTCTTGACTTAAATACTGAAGGATACACTTCTGAGGAAGTTGCTGATAGTAGAATATTGTGC
TTAGCCTTGGTGCATCTTCCTGTTGAAAAGGAAAGCTGGATTGTGTCTGGGACACAGTCTGGTACTCT
ACGCAGTTCACTTTCTAAATGAATCAGGAGTCCTTCTTCATTTTCAAGACCCAGCACTGCAGTTAAGT
GACTTGTACTTTGTGGAACCCAAGTGGCTTTGTAAAATCATGGCACAGATTTTGACAGTGAAAGTGGA
AGGTTGTCCAAAACACCCTAAGGGCATTATTTCGCGTAGAGATGTGGAAAAATTTCTTTCAAAAAAAA
GGAAATTTCCAAAGAACTACATGTCACAGTATTTTAAGCTCCTAGAAAAATTCCAGATTGCTTTGCCA
ATAGGAGAAGAATATTTGCTGGTTCCAAGCAGTTTGTCTGACCACAGGCCTGTGATAGAGCTTCCCCA
TTGTGAGAACTCTGAAATTATCATCCGACTATATGAAATGCCTTATTTTCCAATGGGATTTTGGTCAA
GATTAATCAATCGATTACTTGAGATTTCACCTTACATGCTTTCAGGGAGAGAACGAGCACTTCGCCCA
AACAGAATGTATTGGCGACAAGGCATTTACTTAAATTGGTCTCCTGAAGCTTATTGTCTGGTAGGATC
TGAAGTCTTAGACAATCATCCAGAGAGTTTCTTAAAAATTACAGTTCTTCTTCTTGTAGAAAACTGTA
TTCTTTTGGGCCAAGTTGTGGACCACATTGATTCTCTCATGGAAGAATGGTTTCCTGGGTTGCTGGAG
ATTGATATTTGTGGTGAAGGAGAAACTCTGTTGAAGAAATGGGCATTATATAGTTTTAATGATGGCGA
AGAACATCAAAAAATCTTACTTGATGACTTGATGAAGAAAGCAGAGGAAGGAGATCTCTTAGTAAATC
CAGATCAACCAAGGCTCACCATTCCAATATCTCAGATTGCCCCTGACTTGATTTTGGCTGACCTGCCT
AGAAATATTATGTTGAATAATGATGAGTTGGAATTTGAACAAGCTCCAGAGTTTCTCCTAGGTGATGG
CAGTTTTGGATCAGTTTACCGAGCAGCCTATGAAGGAGAAGAAGTGGCTGTGAAGATTTTTAATAAAC
ATACATCACTCAGGCTGTTAAGACAAGAGCTTGTGGTGCTTTGCCACCTCCACCACCCCAGTTTGATA
TCTTTGCTGGCAGCTGGGATTCGTCCCCGGATGTTGGTGATGGAGTTAGCCTCCAAGGGTTCCTTGGA
TCGCCTGCTTCAGCAGGACAAAGCCAGCCTCACTAGAACCCTACAGCACAGGATTGCACTCCACGTAG
CTGATGGTTTGAGATACCTCCACTCAGCCATGATTATATACCGAGACCTGAAACCCCACAATGTGCTG
CTTTTCACACTGTATCCCAATGCTGCCATCATTGCAAAGATTGCTGACTACGGCATTGCTCAGTACTG
CTGTAGAATGGGGATAAAAACATCAGAGGGCACACCAGGTTTTCGTGCACCTGAAGTTGCCAGAGGAA
ATGTCATTTATAACCAACAGGCTGATGTTTATTCATTTGGTTTACTACTCTATGACATTTTGACAACT
GGAGGTAGAATAGTAGAGGGTTTGAAGTTTCCAAATGAGTTTGATGAATTAGAAATACAAGGAAAATT
ACCTGATCCAGTTAAAGAATATGGTTGTGCCCCATGGCCTATGGTTGAGAAATTAATTAAACAGTGTT
TGAAAGAAAATCCTCAAGAAAGGCCTACTTCTGCCCAGGTATTCGACATTTTGAATTCAGCTGAATTA
CCTGGTCATCAATACCGAAGATGGGAAAAAGAGACATACCCTAGAAAAGATGACTGATTCTGTCACTT
GTTTGTATTGCAATTCCTTTTCCAAGCAAAGCAAACAAAAAAATTTTCTTTTGGTTGGAACCGCTGAT
GGCAAGTTAGCAATTTTTGAAGATAAGACTGTTAAGCTTAAAGGAGCTGCTCCTTTGAAGATACTAAA
TATAGGAAATGTCAGTACTCCATTGATGTGTTTGAGTGAATCCACAAATTCAACGGAAAGAAATGTAA
TGTGGGGAGGATGTGGCACAAAGATTTTCTCCTTTTCTAATGATTTCACCATTCAGAAACTCATTGAG
ACAAGAACAAGCCAACTGTTTTCTTATGCAGCTTTCAGTGATTCCAACATCATAACAGTGGTGGTAGA
CACTGCTCTCTATATTGCTAAGCAAAATAGCCCTGTTGTGGAAGTGTGGGATAAGAAAACTGAAAAAC
TCTGTGGACTAATAGACTGCGTGCACTTTTTAAGGTTAGTAAAACCAAATAGAAAAAAATTATCTAAC
CTTATGATGTCTTTGGCTTTACATCCTATATGTTTAAAATCAAAGTTAAGATGCAGTTCATCCAAAGG
AAGATCCCATATTTTGCTTCGTGTAATTTACAACTTTTGTAATTCGGTCAGAGTCATGATGACAGCAC
AGCTAGGCGGAAGCCTTAAAAATGTCATGCTGGTATTGGGCTACAACCGGAAAAATACTGAAGGTACA
CAAAAGCAGAAAGAGATACAATCTTGCTTGACCGTTTGGGACATCAATCTTCCACATGAAGTGCAAAA
TTTAGAAAAACACATTGAAGTGAGAAAAGAATTAGCTGAAAAAATGAGACGAACATCTGTTGAGTAAG
AGAGAAATAGGAATTGTCTTTGGATAGGAAAATTATTCTCTCCTCTTGTAAATATTTATTTTAAAAAT
GTTCACATGGAAAGGGTACTCACATTTTTTGAAATAGCTCGTGTGTATGAAGGAATGTTATTATTTTT
AATTTAAATATATGTAAAAATACTTACCAGTAAATGTGTATTTTAAAGAACTATTTAAAA

NOV74c, CG90866-01

| | | | |
|---|---|---|---|
| Protein Sequence | SEQ ID NO: 1076 | 915 aa | MW at 103676.4kD |

MAQILTVKVEGCPKHPKGIISRRDVEKFLSKKRKFPKNYMSQYFKLLEKFQIALPIGEEYLLVPSSLS
DHRPVIELPHCENSEIIIRLYEMPYFPMGFWSRLINRLLEISPYMLSGRERALRPNRMYWRQGIYLNW
SPEAYCLVGSEVLDNHPESFLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDLILADLPRNIMLNNDELEFE
QAPEFLLGDGSFGSVYRAAYEGEEVAVKIFNKHTSLRLLRQELVVLCHLHHPSLISLLAAGIRPRMLV
MELASKGSLDRLLQQDKASLTRTLQHRIALHVADGLRYLHSAMIIYRDLKPHNVLLFTLYPNAAIIAK
IADYGIAQYCCRMGIKTSEGTPGFRAPEVARGNVIYNQQADVYSFGLLLYDILTTGGRIVEGLKFPNE
FDELEIQGKLPDPVKEYGCAPWPMVEKLIKQCLKENPQERPTSAQVFSQVFDILNSAELVCLTRRILL
PKNVIVECMVATHHNSRNASIWLGCGHTDRGQLSFLDLNTEGYTSEEVADSRILCLALVHLPVEKESW
IVSGTQSGTLLVINTEDGKKRHTLEKMTDSVTCLYCNSFSKQSKQKNFLLVGTADGKLAIFEDKTVKL
KGAAPLKILNIGNVSTPLMCLSESTNSTERNVMWGGCGTKIFSFSNDFTIQKLIETRTSQLFSYAAFS
DSNIITVVVDTALYIAKQNSPVVEVWDKTEKLCGLIDCVHFLRLVKPNRKKLSNLMMSLALHPICKLK
SKLRCSSSKGRSHILLRVIYNFCNSVRVMMTAQLGGSLKNVMLVLGYNRKNTEGTQKQKEIQSCLTVW
DINLPHEVQNLEKHIEVRKELAEKMRTSVE

| | | |
|---|---|---|
| NOV74d, CG90866-02 | SEQ ID NO: 1077 | 3040 bp |

| | | |
|---|---|---|
| DNA Sequence | ORF Start: ATG at 108 | ORF Stop: TAA at 2841 |

ACGCAGTTCACTTTCTAAATGAATCAGGAGTCCTTCTTCATTTTCAAGACCCAGCACTGCAGTTAAGT
GACTTGTACTTTGTGGAACCCAAGTGGCTTTGTAAAATCATGGCACAGATTTTGACAGTGAAAGTGGA
AGGTTGTCCAAAACACCCTAAGGGCATTATTTCGCGTAGAGATGTGGAAAAATTTCTTTCAAAAAAAA
GGAAATTTCCAAAGAACTACATGTCACAGTATTTTAAGCTCCTAGAAAAATTCCAGATTGCTTTGCCA
ATAGGAGAAGAATATTTGCTGGTTCCAAGCAGTTTGTCTGACCACAGGCCTGTGATAGAGCTTCCCCA
TTGTGAGAACTCTGAAATTATCATCCGACTATATGAAATGCCTTATTTTCCAATGGGATTTTGGTCAA
GATTAATCAATCGATTACTTGAGATTTCACCTTACATGCTTTCAGGGAGAGAACGAGCACTTCGCCCA
AACAGAATGTATTGGCGACAAGGCATTTACTTAAATTGGTCTCCTGAAGCTTATTGTCTGGTAGGATC
TGAAGTCTTAGACAATCATCCAGAGAGTTTCTTAAAAATTACAGTTCTTCTTCTTGTAGAAAACTGTA
TTCTTTTGGGCCAAGTTGTGGACCACATTGATTCTCTCATGGAAGAATGGTTTCCTGGGTTGCTGGAG
ATTGATATTTGTGGTGAAGGAGAAACTCTGTTGAAGAAATGGGCATTATATAGTTTTAATGATGGCGA
AGAACATCAAAAAATCTTACTTGATGACTTGATGAAGAAAGCAGAGGAAGGAGATCTCTTAGTAAATC
CAGATCAACCAAGGCTCACCATTCCAATATCTCAGATTGCCCCTGACTTGATTTTGGCTGACCTGCCT
AGAAATATTATGTTGAATAATGATGAGTTGGAATTTGAACAAGCTCCAGAGTTTCTCCTAGGTGATGG
CAGTTTTGGATCAGTTTACCGAGCAGCCTATGAAGGAGAAGAAGTGGCTGTGAAGATTTTTAATAAAC
ATACATCACTCAGGCTGTTAAGACAAGAGCTTGTGGTGCTTTGCCACCTCCACCACCCCAGTTTGATA
TCTTTGCTGGCAGCTGGGATTCGTCCCCGGATGTTGGTGATGGAGTTAGCCTCCAAGGGTTCCTTGGA
TCGCCTGCTTCAGCAGGACAAAGCCAGCCTCACTAGAACCCTACAGCACAGGATTGCACTCCACGTAG
CTGATGGTTTGAGATACCTCCACTCAGCCATGATTATATACCGAGACCTGAAACCCCACAATGTGCTG
CTTTTCACACTGTATCCCAATGCTGCCATCATTGCAAAGATTGCTGACTACGGCATTGCTCAGTACTG
CTGTAGAATGGGGATAAAAACATCAGAGGGCACACCAGGTTTTCGTGCACCTGAAGTTGCCAGAGGAA
ATGTCATTTATAACCAACAGGCTGATGTTTATTCATTTGGTTTACTACTCTATGACATTTTGACAACT
GGAGGTAGAATAGTAGAGGGTTTGAAGTTTCCAAATGAGTTTGATGAATTAGAAATACAAGGAAAATT
ACCTGATCCAGTTAAAGAATATGGTTGTGCCCCATGGCCTATGGTTGAGAAATTAATTAAACAGTGTT
TGAAAGAAAATCCTCAAGAAAGGCCTACTTCTGCCCAGGTATTCGACATTTTGAATTCAGCTGAATTA
GTCTGTCTGACGAGACGCATTTTATTACCTAAAAACGTAATTGTTGAATGCATGGTTGCTACACATCA
CAACAGCAGGAATGCAAGCATTTGGCTGGGCTGTGGGCACACCGACAGAGGACAGCTCTCATTTCTTG
ACTTAAATACTGAAGGATACACTTCTGAGGAAGTTGCTGATAGTAGAATATTGTGCTTAGCCTTGGTG
CATCTTCCTGTTGAAAAGGAAAGCTGGATTGTGTCTGGGACACAGTCTGGTACTCTCCTGGTCATCAA
TACCGAAGATGGGAAAAAGAGACATACCCTAGAAAAGATGACTGATTCTGTCACTTGTTTGTATTGCA
ATTCCTTTTCCAAGCAAAGCAAACAAAAAAATTTTCTTTTGGTTGGAACCGCTGATGGCAAGTTAGCA
ATTTTTGAAGATAAGACTGTTAAGCTTAAAGGAGCTGCTCCTTTGAAGATACTAAATATAGGAAATGT
CAGTACTCCATTGATGTGTTTGAGTGAATCCACAAATTCAACGGAAAGAAATGTAATGTGGGGAGGAT
GTGGCACAAAGATTTTCTCCTTTTCTAATGATTTCACCATTCAGAAACTCATTGAGACAAGAACAAGC
CAACTGTTTTCTTATGCAGCTTTCAGTGATTCCAACATCATAACAGTGGTGGTAGACACTGCTCTCTA
TATTGCTAAGCAAAATAGCCCTGTTGTGGAAGTGTGGGATAAGAAAACTGAAAAACTCTGTGGACTAA
TAGACTGCGTGCACTTTTTAAGCCTTAAAAATGTCATGCTGGTATTGGGCTACAACCGGAAAAATACT
TTGGCTTTACATCCTATATGTTTAAAATCAAAGTTAAGATGCAGTTCATCCAAAGGAAGATCCCATAT
TTTGCTTCGTGTAATTTACAACTTTTGTAATTCGGTCAGAGTCATGATGACAGCACAGCTAGGCGGAA
GCCTTAAAAATGTCATGCTGGTATTGGGCTACAACCGGAAAAATACTGAAGGTACACAAAAGCAGAAA
GAGATACAATCTTGCTTGACCGTTTGGGACATCAATCTTCCACATGAAGTGCAAAATTTAGAAAAACA
CATTGAAGTGAGAAAAGAATTAGCTGAAAAAATGAGACGAACATCTGTTGAGTAAGAGAGAAATAGGA
ATTGTCTTTGGATAGGAAAATTATTCTCTCCTCTTGTAAATATTTATTTTAAAAATGTTCACATGGAA
AGGGTACTCACATTTTTTGAAATAGCTCGTGTGTATGAAGGAATGTTATTATTTTTAATTTAAATATA
TGTAAAAATACTTACCAGTAAATGTGTATTTTAAAGAACTATTTAAAA

NOV74d, CG90866-02

| | | | |
|---|---|---|---|
| Protein Sequence | SEQ ID NO: 1078 | 911 aa | MW at 103214.9kD |

MAQILTVKVEGCPKHPKGIISRRDVEKFLSKKRKFPKNYMSQYFKLLEKFQIALPIGEEYLLVPSSLS
DHRPVIELPHCENSEIIIRLYEMPYFPMGFWSRLINRLLEISPYMLSGRERALRPNRMYWRQGIYLNW
SPEAYCLVGSEVLDNHPESFLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDLILADLPRNIMLNNDELEFE
QAPEFLLGDGSFGSVYRAAYEGEEVAVKIFNKHTSLRLLRQELVVLCHLHHPSLISLLAAGIRPRMLV
MELASKGSLDRLLQQDKASLTRTLQHRIALHVALGLRYLHSAMIIYRDLKPHNVLLFTLYPNAAIIAK
IADYGIAQYCCRMGIKTSEGTPGFRAPEVARGNVIYNQQADVYSFGLLLYDILTTGGRIVEGLKFPNE
FDELEIQGKLPDPVKEYGCAPWPMVEKLIKQCLKENPQERPTSAQVFDILNSAELVCLTRRILLPKNV
IVECMVATHHNSRNASIWLGCGHTDRGQLSFLDLNTEGYTSEEVADSRILCLALVHLPVEKESWIVSG
TQSGTLLVINTEDGKKRHTLEKMTDSVTCLYCNSFSKQSKQKNFLLVGTADGKLAIFEDKTVKLKGAA
PLKILNIGNVSTPLMCLSESTNSTERNVMWGGCGTKIFSFSNDFTIQKLIETRTSQLFSYAAFSDSNI
ITVVVDTALYIAKQNSPVVEVWDKKTEKLCGLIDCVHFLRLVKPNRKKLSNLMMSLALHPICLKSKLR
CSSSKGRSHILLRVIYNFCNSVRVMMTAQLGGSLKNVMLVLGYNRKNTEGTQKQKEIQSCLTVWDINL
PHEVQNLEKHIEVRKELAEKMRRTSVE

| | | |
|---|---|---|
| NOV74e, CG90866-05 | SEQ ID NO: 1079 | 2955 bp |

| | | |
|---|---|---|
| DNA Sequence | ORF Start: ATG at 81 | ORF Stop: TAA at 2844 |

GAGTCCTTCTTCATTTTCAAGACCCAGCACTGCAGTTAAGTGACTTGTACTTTGTGGAACCCAAGTGG
CTTTGTAAAATCATGGCACAGATTTTGACAGTGAAAGTGGAAGGTTGTCCAAAACACCCTAAGGGAAT
TATTTCGCGTAGAGATGTGGAAAAATTTCTTTCAAAAAAAAGGAAATTTCCAAAGAACTACATGTCAC
AGTATTTTAAGCTCCTAGAAAAATTCCAGATTGCTTTGCCAATAGGAGAAGAATATTTGCTGGTTCCA
AGCAGTTTGTCTGACCACAGGCCTGTGATAGAGCTTCCCCATTGTGAGAACTCTGAAATTATCATCCG
AGCAGTTTGTCTGACCACAGGCCTGTGATAGAGCTTCCCCATTGTGAGAACTCTGAAATTATCATCCG
ACTATATGAAATGCCTTATTTTCCAATGGGATTTTGGTCAAGATTAATCAATCGATTACTTGAGATTT
CACCTTACATGCTTTCAGGGAGAGAACGAGCACTTCGCCCAAACAGAATGTATTGGCGACAAGGCATT
TACTTAAATTGGTCTCCTGAAGCTTATTGTCTGGTAGGATCTGAAGTCTTAGACAATCATCCAGAGAG
TTTCTTAAAAATTACAGTTCCTTCTTGTAGAAAAGGCTGTATTCTTTTGGGCCAAGTTGTGGACCACA
TTGATTCTCTCATGGAAGAATGGTTTCCTGGGTTGCTGGAGATTGATATTTGTGGTGAAGGAGAAACT
CTGTTGAAGAAATGGGCATTATATAGTTTTAATGATGGTGAAGAACATCAAAAAATCTTACTTGATGA
CTTGATGAAGAAAGCAGAGGAAGGAGATCTCTTAGTAAATCCAGATCAACCAAGGCTCACCATTCCAA
TATCTCAGATTGCCCCTGACTTGATTTTGGCTGACCTGCCTAGAAATATTATGTTGAATAATGATGAG
TTGGAATTTGAACAAGCTCCAGAGTTTCTCCTAGGTGATGGCAGTTTTGGATCAGTTTACCGAGCAGC
CTATGAAGGAGAAGAAGTGGCTGTGAAGATTTTTAATAAACATACATCACTCAGGCTGTTAAGACAAG
AGCTTGTGGTGCTTTGCCACCTCCACCACCCCAGTTTGATATCTTTGCTGGCAGCTGGGATTCGTCCC
CGGATGTTGGTGATGGAGTTAGCCTCCAAGGGTTCCTTGGATCGCCTGCTTCAGCAGGACAAAGCCAG
CCTCACTAGAACCCTACAGCACAGGATTGCACTCCACGTAGCTGATGGTTTGAGATACCTCCACTCAG
CCATGATTATATACCGAGACCTGAAACCCCACAATGTGCGGCTTTTCACACTGTATCCCAATGCTGCC
ATCATTGCAAAGATTGCTGACTACGGCATTGCTCAGTACTGCTGTAGAATGGGGATAAAAACATCAGA
GGGCACACCAGGGTTTCGTGCACCTGAAGTTGCCAGAGGAAATGTCATTTATAACCAACAGGCTGATG
TTTATTCATTTGGTTTACTACTCTATGACATTTTGACAACTGGAGGTAGAATAGTAGAGGGTTTGAAG
TTTCCAAATGAGTTTGATGAATTAGAAATACAAGGAAAATTACCTGATCCAGTTAAAGAATATGGTTG
TGCCCCATGGCCTATGGTTGAGAAATTAATTAAACAGTGTTTGAAAGAAAATCCTCAAGAAAGGCCTA
CTTCTGCCCAGGTCTTTGACATTTTGAATTCAGCTGATTAGTCTGTCTGACGAGACGCATTTTTATTA
CCTAAAAACGTAATTGTTGAATGCATGGTTGCTACACATCACAACAGCAGGAATGCAAGCATTTGGCT
GGGCTGTGGGCACACCGACAGAGGACAGCTCTCATTTCTTGACTTAAATACTGAAGGATACACTTCTG
AGGAAGTTGCTGATAGTAGAATATTGTGCTTAGCCTTGGTGCATCTTCCTGTTGAAAAGGAAAGCTGG
ATTGTGTCTGGGACACAGTCTGGTACTCTCCTGGTCATCAATACCGAAGATGGGAAAAAGAGACATAC
CCTAGAAAAGATGACTGATTCTGTCACTTGTTTGTATTGCAATTCCTTTTCCAAGCAAAGCAAACAAA
AAAATTTTCTTTTGGTTGGAACCGCTGATGGCAAGTTAGCAATTTTTGAAGATAAGACTGTTAAGCTT
AAAGGAGCTGCTCCTTTGAAGATACTAAATATAGGAATGTCAGGTACTCCATTGATGTGTTTGAGTGA
ATCCACAAATTCAACGGAAAGAAATGTAATGTGGGGAGGATGTGGCACAAAGATTTTCTCCTTTTCTA
ATGATTTCACCATTCAGAAACTCATTGAGACAAGAACAAGCCAACTGTTTTCTTATGCAGCTTTCAGT
GATTCCAACATCATAACAGTGGTGGTAGACACTGCTCTCTATATTGCTAAGCAAAATAGCCCTGTTGT
GGAAGTGTGGGATAAGAAAACTGAAAAACTCTGTGGGCTAATAGACTGCGTGCACTTTTTAAGGGAGG
TAACGGTAAAAGAAAACAAGGAATCAAAACACAAAATGTCTTATTCTGGGAGAGTGAAAACCCTCTGC
CTTCAGAAGAACACTGCTCTTTGGATAGGAACTGGAGGAGGCCATATTTTACTCCTGGATCTTTCAAC
TCGTCGACTTATACGTGTAATTTACAACTTTTGTAATTCGGTCAGAGTCATGATGACAGCACAGCTAG
GAAGCCTTAAAAATGTCATGCTGGTATTGGGCTACAACCGGAAAAATACTGAAGGTACACAAAAGCAG
AAAGAGATACAATCTTGCTTGACCGTTTGGGACATCAATCTTCCACATGAAGTGCAAAATTTAGAAAA
ACACATTGAAGTGAGAAAAGAATTAGCTGAAAAAATGAGACGAACATCTGTTGAGTAAGAGAGAAATA
GGAATTGTCTTTGGATAGGAAAATTATTCTCTCCTCTTGTAAATATTTATTTTAAAAATGTTCACATG
GAAAGGGTACTCACATTTTTAAGGGCGAATC

NOV74e, CG90866-05

| | | | |
|---|---|---|---|
| Protein Sequence | SEQ ID NO: 1080 | 921 aa | MW at 104423.0kD |

MAQILTVKVEGCPKHPKGIISRRDVEKFLSKKRKFPKNYMSQYFKLLEKFQIALPIGEEYLLVPSSLS
DHRPVIELPHCENSEIIIRLYEMPYFPMGFWSRLINRLLEISPYMLSGRERALRPNRMYWRQGIYLNW
SPEAYCLVGSEVLDNHPESFLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDLILADLPRNIMLNNDELEFE
QAPEFLLGDGSFGSVYRAAYEGEEVAVKIFNKHTSLRLLRQELVVLCHLHHPSLISLLAAGIRPRMLV
MELASKGSLDRLLQQDKASLTRTLQHRIALHVALGLRYLHSAMIIYRDLKPHNVLLFTLYPNAAIIAK
IADYGIAQYCCRMGIKTSEGTPGFRAPEVARGNVIYNQQADVYSFGLLLYDILTTGGRIVEGLKFPNE
FDELEIQGKLPDPVKEYGCAPWPMVEKLIKQCLKENPQERPTSAQVFDILNSAELVCLTRRILLPKNV
IVECMVATHHNSRNASIWLGCGHTDRGQLSFLDLNTEGYTSEEVADSRILCLALVHLPVEKESWIVSG
TQSGTLLVINTEDGKKRHTLEKMTDSVTCLYCNSFSKQSKQKNFLLVGTADGKLAIFEDKTVKLKGAA
PLKILNIGNVSTPLMCLSESTNSTERNVMWGGCGTKIFSFSNDFTIQKLIETRTSQLFSYAAFSDSNI
ITVVVDTALYIAKQNSPVVEVWDKKTEKICGLIDCVHFLSLKNVMLVLGYNRKNTEGTQKQKEIQSCL
TALWIGTGGGHILLLDLSTRRLIRVIYNFCNSVRVMMTAQLGSLKNVMLVLGYNRKNTEGTQKQKEIQ
SCLTVWDINLPHEVQNLEKMIEVRKELAEKMRRTSVE

A ClustalW comparison of the above protein sequences yields the following sequence alignment shown in Table 74B.

TABLE 74B
Comparison of the NOV74 protein sequences.

| | |
|---|---|
| NOV74a | MQPLDFSSGGSDPNISLSEKIRDQLVVGQLIPDCYVELEKIILSERKNVPIEFPVIDRKR |
| NOV74b | ------------------------------------------------------------ |
| NOV74c | ------------------------------------------------------------ |
| NOV74d | ------------------------------------------------------------ |
| NOV74e | ------------------------------------------------------------ |
| NOV74a | LLQLVRENQLQLDENELPHAVHFLNESGVLLHFQDPALQLSKLYFVEPKWLCKIMAQDVS |
| NOV74b | ------------------------------------------------------MAQ--- |
| NOV74c | ------------------------------------------------------MAQ--- |
| NOV74d | ------------------------------------------------------MAQ--- |
| NOV74e | ------------------------------------------------------MAQ--- |
| NOV74a | SIFGLYIRDILTVKVEGCPKHPKGIISRRDVEKFLSKKRKFPKNYMSQYFKLLEKFQIAL |
| NOV74b | ---------ILTVKVEGCPKHPKGIISRRDVEKFLSKKRKFPKMYMSQYFKLLEKFQIAL |
| NOV74c | ---------ILTVKVEGCPKHPKGIISRRDVEKFLSKKRKFPKMYMSQYFKLLEKFQIAL |
| NOV74d | ---------ILTVKVEGCPKHPKGIISRRDVEKFLSKKRKFPKMYMSQYFKLLEKFQIAL |
| NOV74e | ---------ILTVKVEGCPKHPKGIISRRDVEKFLSKKRKFPKMYMSQYFKLLEKFQIAL |
| NOV74a | PIGEEYLLVPSSLSDHRPVIELPHCENSEIIIRLYEMPYFPMGFWSRLINRLLEISPYML |
| NOV74b | PIGEEYLLVPSSLSDHRPVIELPHCENSEIIIRLYEMPYFPMGFWSRLINRLLEISPYML |
| NOV74c | PIGEEYLLVPSSLSDHRPVIELPHCENSEIIIRLYEMPYFPMGFWSRLINRLLEISPYML |
| NOV74d | PIGEEYLLVPSSLSDHRPVIELPHCENSEIIIRLYEMPYFPMGFWSRIINRLLEISPYML |
| NOV74e | PIGEEYLLVPSSLSDHRPVIELPHCENSEIIIRLYEMPYFPMGFWSRIINRLLEISPYML |
| NOV74a | SG--R-----------------------------------------------GCILLGQV |
| NOV74b | SGRERALRPNRMYWRQGIYLNWSPEAYCLVGSEVLDNHPESFLKITVPSCRKGCILLGQV |
| NOV74c | SGRERALRPNRMYWRQGIYLNWSPEAYCLVGSEVLDNHPESFLKITVPSCRKGCILLGQV |
| NOV74d | SGRERALRPNRNYWRQGIYLNWSPEAYCLVGSEVLDNHPESFLKITVPSCRKGCILLGQV |
| NOV74e | SGRERALRPNRMYWRQGIYLNWSPEAYCLVGSEVLDNHPESFLKITVPSCRKGCILLGQV |
| NOV74a | VDHIDSLMEEWFPGLLEIDICGEGETLLKKWALYSFNDGEEHQKILLDDLMKKAEEGDLL |
| NOV74b | VDHIDSLMEEWFPGLLEIDICGEGETLLKKWALYSFNDGEEHQKILLDDLMKKAEEGDLL |
| NOV74c | VDHIDSLMEEWFPGLLEIDICGEGETLLKKWALYSFNDGEEHQKILLDDLMKKAEEGDLL |
| NOV74d | VDHIDSLMEEWFPGLLEIDICGEGETLLKKWALYSFNDGEEHQKILLDDLMKKAEEGDLL |
| NOV74e | VDHIDSLMEEWFPGLLEIDICGEGETLLKKWALYSFNDGEEHQKILLDDLMKKAEEGDLL |
| NOV74a | VNPDQPRLTIPISQIAPDLILADPPRNIMLNNDELEFEQAPEFLLDCFVCIHLYPSSDYI |
| NOV74b | VNPDQPRLTIPISQIAPDLILADLPRNIMLNNDELEFEQAPEFLL--------------- |
| NOV74c | VNPDQPRLTIPISQIAPDLILADLPRNIMLNNDELEFEQAPEFLL--------------- |
| NOV74d | VNPDQPRLTIPISQIAPDLILADLPRNIMLNNDELEFEQAPEFLL--------------- |
| NOV74e | VNPDQPRITIPISQIAPDLILADLPRNIMLNNDELEFEQAPEFLL--------------- |
| NOV74a | SRHYMRTINIVQTGFAKCRWRVTVHGADHGDGSFGSVYRAAYEGEEVAVKIFNKHTSLRL |
| NOV74b | -----------------------------GDGSFGSVYRAAYEGEEVAVKIFNKHTSLRL |
| NOV74c | -----------------------------GDGSFGSVYRAAYEGEEVAVKIFNKHTSLRL |
| NOV74d | -----------------------------GDGSFGSVYRAAYEGEEVAVKIFNKHTSLRL |
| NOV74e | -----------------------------GDGSFGSVYRAAYEGEEVAVKIFNKHTSLRL |
| NOV74a | LRQELVVLCHLHHPSLISLLAAGIRPRMLVMELASKGSLDRLLQQDKASLTRTLQHRIAL |
| NOV74b | LRQELVVLCHLHHPSLISLLAAGIRPRMLVMELASKGSLDRLLQQDKASLTRTLQHRIAL |
| NOV74c | LRQELVVLCHLHHPSLISLLAAGIRPRMLVMELASKGSLDRLLQQDKASLTRTLQHRIAL |
| NOV74d | LRQELVVLCHLHHPSLISLLAAGIRPRMLVMELASKGSLDRLLQQDKASLTRTLQHRIAL |
| NOV74e | LRQELVVLCHLHHPSLISLLAAGIRPRMLVMELASKGSLDRLLQQDKASLTRTLQHRIAL |
| NOV74a | HVADGLRYLHSAMIIYRDLKPHNVLLFTLYPNAAIIAKIADYGIAQYCCRMGIKTSEGTP |
| NOV74b | HVADGLRYLHSAMIIYRDLKPHNVLLFTLYPNAAIIAKIADYGIAQYCCRMGIKTSEGTP |
| NOV74c | HVADGLRYLHSANIIYRDLKPHNVLLFTLYPNAAIIAKIADYGIAQYCCRMGIKTSEGTP |
| NOV74d | HVADGLRYLHSANIIYRDLKPHNVLLFTLYPNAAIIAKIADYGIAQYCCRMGIKTSEGTP |
| NOV74e | HVADGLRYLHSAMIIYRDLKPHNVRLFTLYPNAAIIAKIADYGIAQYCCRNGIKTSEGTP |
| NOV74a | GFRAPEVARGNVIYNQQADVYSFGLLLYDILTTGGRIVEGLKFPNEFDELEIQGKLPDPV |
| NOV74b | GFRAPEVARGNVIYNQQADVYSFGLLLYDILTTGGRIVEGLKFPNEFDELEIQGKLPDPV |
| NOV74c | GFRAPEVARGNVIYNQQADVYSFGLLLYDILTTGGRIVEGLKFPNEFDELEIQGKLPDPV |
| NOV74d | GFRAPEVARGNVIYNQQADVYSFGLLLYDILTTGGRIVEGLKFPNEFDELEIQGKLPDPV |
| NOV74e | GFRAPEVARGNVIYNQQADVYSFGLLLYDILTTGGRIVEGLKFPNEFDELEIQGKLPDPV |
| NOV74a | KEYGCAPWPMVEKLIKQCLKENPQERPTSAQVF----DILNSAELVCLTRRILLPKNVIV |
| NOV74b | KEYGCAPWPMVEKLIKQCLKENPQERPTSAQVF----DILNSAELVCLTRRILLPKNVIV |
| NOV74c | KEYGCAPWPMVEKLIKQCLKENPQERPTSAQVFSQVFDILNSAELVCLTRRILLPKNVIV |
| NOV74d | KEYGCAPWPMVEKLIKQCLKENPQERPTSAQVF----DILNSAELVCLTRRILLPKNVIV |
| NOV74e | KEYGCAPWPMVEKLIKQCLKENPQERPTSAQVF----DILNSAELVCLTRRILLPKNVIV |
| NOV74a | ECMVATHHNSRNASIWLGCGHTDRGQLSFLDLNTEGYTSE-------------------- |
| NOV74b | ECMVATHHNSRNASIWLGCGHTDRGQLSFLDLNTEGYTSEEVADSRILCLALVHLPVEKE |
| NOV74c | ECMVATHHNSRNASIWLGCGHTDRGQLSFLDLNTEGYTSEEVADSRILCLALVHLPVEKE |
| NOV74d | ECMVATHHNSRNASIWLGCGHTDRGQLSFLDLNTEGYTSEEVADSRILCLALVHLPVEKE |
| NOV74e | ECMVATHHNSRNASIWLGCGHTDRGQLSFLDLNTEGYTSEEVADSRILCLALVHLPVEKE |
| NOV74a | --------------------------------------------SKQKNFLLVGTADGKL |
| NOV74b | SWIVSGTQSGTLLVINTEDGKKRHTLEKMTDSVTCLYCNSFSKQSKQKNFLLVGTADGKL |
| NOV74c | SWIVSGTQSGTLLVINTEDGKKRHTLEKMTDSVTCLYCNSFSKQSKQKNFLLVGTADGKL |
| NOV74d | SWIVSGTQSGTLLVINTEDGKKRHTLEKMTDSVTCLYCNSFSKQSKQKNFLLVGTADGKL |
| NOV74e | SWIVSGTQSGTLLVINTEDGKKRHTLEKMTDSVTCLYCNSFSKQSKQKNFLLVGTADGKL |
| NOV74a | AIFEDKTVKLKGAAPLKILNIGNVSTPLMCLSESTNSTERNVMWGGCGTKIFSFSNDFTI |
| NOV74b | AIFEDKTVKLKGAAPLKILNIGNVSTPLMCLSESTNSTERNVMWGGCGTKIFSFSNDFTI |
| NOV74c | AIFEDKTVKLKGAAPLKILNIGNVSTPLMCLSESTNSTERNVMWGGCGTKIFSFSNDFTI |
| NOV74d | AIFEDKTVKLKGAAPLKILNIGNVSTPLMCLSESTNSTERNVMWGGCGTKIFSFSNDFTI |
| NOV74e | AIFEDKTVKLKGAAPLKILNIGNVSTPLMCLSESTNSTERNVMWGGCGTKIFSFSNDFTI |
| NOV74a | QKLIETRTSQLFSYAAFSDSNIITVVVDTALYIAKQNSPVVEVWDKKTEKLCGLIDCVHF |
| NOV74b | QKLIETRTSQLFSYAAFSDSNIITVVVDTALYIAKQNSPVVEVWDKKTEKLCGLIDCVHF |
| NOV74c | QKLIETRTSQLFSYAAFSDSNIITVVVDTALYIAKQNSPVVEVWDKKTEKLCGLIDCVHF |
| NOV74d | QKLIETRTSQLFSYAAFSDSNIITVVVDTALYIAKQNSPVVEVWDKKTEKLCGLIDCVHF |
| NOV74e | QKLIETRTSQLFSYAAFSDSNIITVVVDTALYIAKQNSPVVEVWDKKTEKLCGLIDCVHF |
| NOV74a | LREVMVKENKESKHKMSYSGRVKTLCLQKNTALWIGTGGGHILLLDLSTRRLIRVIYNFC |
| NOV74b | L----------S------------------------------------------------ |
| NOV74c | LRLVKPNRKKLSNLMMSLA--LHPICLKSKLRCSSSKGRSHILL---------RVIYNFC |
| NOV74d | LRLVKPNRKKLSNLMMSLA--LHPICLKSKLRCSSSKGRSHILL---------RVIYNFC |
| NOV74e | LREVMVKENKESKHKMSYSGRVKTLCLQKNTALWIGTGGGHILLLDLSTRRLIRVIYNFC |
| NOV74a | NSVRVMMTAQLG-SLKNVMLVLGYNRKNTEGTQKQKEIQSCLTVWDINLPHEVQNLEKHI |
| NOV74b | --------------LKNVMLVLGYNRKNTEGTQKQKEIQSCLTVWDINLPHEVQNLEKHI |
| NOV74c | NSVRVMMTAQLGGSLKNVMLVLGYNRKNTEGTQKQKEIQSCLTVWDINLPHEVQNLEKHI |
| NOV74d | NSVRVMMTAQLGGSLKNVMLVLGYNRKNTEGTQKQKEIQSCLTVWDINLPHEVQNLEKHI |
| NOV74e | NSVRVMMTAQLG-SLKNVMLVLGYNRKNTEGTQKQKEIQSCLTVWDINLPHEVQNLEKHI |
| NOV74a | EVRKELAEKMRRTSVE |
| NOV74b | EVRKELAEKMRRTSVE |
| NOV74c | EVRKELAEKMRRTSVE |
| NOV74d | EVRKELAEKMRRTSVE |
| NOV74e | EVRKELAEKMRRTSVE |
| NOV74a | (SEQ ID NO: 1072) |
| NOV74b | (SEQ ID NO: 1074) |
| NOV74c | (SEQ ID NO: 1076) |
| NOV74d | (SEQ ID NO: 1078) |
| NOV74e | (SEQ ID NO: 1080) |

Further analysis of the NOV74a protein yielded the following properties shown in Table 74C.

TABLE 74C
Protein Sequence Properties NOV74a

| | |
|---|---|
| SignalP analysis: | No Known Signal Sequence Indicated |

PSORT II analysis:

| PSG: | a new signal peptide prediction method | | | |
|---|---|---|---|---|
| | N-region: | length 5; | pos.chg 0; | neg.chg 1 |
| | H-region: | length 6; | peak value | 0.00 |
| | PSG score: | −4.40 | | |

| | |
|---|---|
| GvH: | von Heijne's method for signal seq. recognition |
| | GvH score (threshold: −2.1): −11.05 |
| | possible cleavage site: between 13 and 14 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 2 |
| | Number of TMS(s) for threshold 0.5: 0 |
| | PERIPHERAL Likelihood = 1.96 (at 794) |
| | ALOM score: −0.64 (number of TMSs: 0) |

MITDISC: discrimination of mitochondrial targeting seq

| | | | |
|---|---|---|---|
| R content: | 0 | Hyd Moment (75): | 1.98 |
| Hyd Moment(95): | 4.87 | G content: | 2 |
| D/E content: | 2 | S/T content: | 3 |
| Score: −7.81 | | | |

Gavel: prediction of cleavage sites for mitochondrial preseq

cleavage site motif not found

NUCDISC: discrimination of nuclear localization signals

pat4: KKRK (5) at 157
pat7: PVIDRKR (3) at 54
bipartite: RKNVPIEFPVIDRKRLL at 46
content of basic residues: 11.6%
NLS Score: 0.59

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): ***found***
LQLDENELPHAVHFLNESGVLL at 70
Regulator of chromosome condensation (RCC1) signature 2

(PS00626): ***found***

IGTGGGHILLL at 878

checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
  52.2%: cytoplasmic
  39.1%: nuclear
    8.7%: mitochondrial
```

>> prediction for CG90866-04 is cyt (k = 23)

A search of the NOV74a protein against the Geneseq database, a proprietary database that contains sequences published in patents and patent publication, yielded several homologous proteins shown in Table 74D.

TABLE 74D
Geneseq Results for NOV74a

| | | NOV74a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU03554 | Human protein kinase #54 | 1 . . . 845 | 835/909 (91%) | 0.0 |
| | Homo sapiens, 909 aa. | 1 . . . 909 | 839/909 (91%) | |
| | [WO200138503-A2, 31-MAY- | | | |
| | 2001] | | | |
| ABU11054 | Human protein NOV19b - Homo | 130 . . . 978 | 754/963 (78%) | 0.0 |
| | sapiens, 911 aa. [WO200281629- | 4 . . . 911 | 765/963 (79%) | |
| | A2, 17-OCT-2002] | | | |
| ABU11053 | Human protein NOV19a - Homo | 130 . . . 978 | 754/967 (77%) | 0.0 |
| | sapiens, 915 aa. [WO200281629- | 4 . . . 915 | 765/967 (78%) | |
| | A2, 17-OCT-2002] | | | |
| AAE16259 | Human kinase PKIN-5 protein - | 401 . . . 978 | 567/642 (88%) | 0.0 |
| | Homo sapiens, 656 aa. | 15 . . . 656 | 571/642 (88%) | |
| | [WO200196547-A2, 20-DEC- | | | |
| | 2001] | | | |
| AAU11287 | Human transducin polypeptide | 670 . . . 978 | 298/373 (79%) | e−162 |
| | 41 - Homo sapiens, 373 aa. | 1 . . . 373 | 302/373 (80%) | |
| | [CN1306988-A, 08-AUG-2001] | | | |

In a BLAST search of public sequence databases, the NOV74a protein was found to have homology to the proteins shown in the BLASTP data in Table 74E.

TABLE 74E
Public BLASTP Results for NOV74a

| | | NOV74a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8K062 | Similar to RIKEN cDNA | 208 . . . 978 | 642/884 (72%) | 0.0 |
| | 4921513O20 gene - Mus | 1 . . . 840 | 694/884 (77%) | |
| | musculus (Mouse), 840 aa | | | |
| | (fragment). | | | |
| Q9CQG8 | 4921513O20Rik protein - Mus | 499 . . . 978 | 401/545 (73%) | 0.0 |
| | musculus (Mouse), 561 aa. | 18 . . . 561 | 441/545 (80%) | |
| Q8NCX9 | Hypothetical protein - Homo | 643 . . . 978 | 326/400 (81%) | e−178 |
| | sapiens (Human), 400 aa | 1 . . . 400 | 330/400 (82%) | |
| | (fragment). | | | |
| Q8CI84 | Hypothetical protein - Mus | 669 . . . 978 | 239/329 (72%) | e−133 |
| | musculus (Mouse), 327 aa | 1 . . . 327 | 269/329 (81%) | |
| | (fragment). | | | |
| Q8BZJ6 | Hypothetical serine/threonine | 14 . . . 224 | 183/211 (86%) | 1e−99 |
| | protein kinase containing protein - | 345 . . . 543 | 190/211 (89%) | |
| | Mus musculus (Mouse), 546 aa. | | | |

PFam analysis indicates that the NOV74a protein contains the domains shown in the Table 74F.

TABLE 74F
Domain Analysis of NOV74a

| | NOV74a | Identities/Similarities | Expect |
|---|---|---|---|
| Pfam Domain | Match Region | for the Matched Region | Value |
| pkinase | 394 . . . 644 | 90/302 (30%) | 1.4e−33 |
| | | 169/302 (56%) | |

### Example 75

The NOV75 clone was analyzed, and the nucleotide and encoded polypeptide sequences are shown in Table 75A.

TABLE 75A
NOV75 Sequence Analysis

| | | |
|---|---|---|
| NOV75a, CG91708-02 | SEQ ID NO: 1081 | 1580 bp |

| | | |
|---|---|---|
| DNA Sequence | ORF Start: ATG at 51 | ORF Stop: TGA at 1482 |

CAAGACAGCAAGGCATAGAGACAACATAGAGCTAAGTAAAGCCAGTGGAAATGAAGAGTCTTCCAATC
CTACTGTTGCTGTGCGTGGCAGTTTGCTCAGCCTATCCATTGGATGGAGCTGCAAGGGGTGAGGACAC
CAGCATGAACCTTGTTCAGAAATATCTAGAAAACTACTACGACCTCGAAAAAGATGTGAAACAGTTTG
TTAGGAGAAAGGACAGTGGTCCTGTTGTTAAAAAAATCCGAGAAATGCAGAAGTTCCTTGGATTGGAG
GTGACGGGGAAGCTGGACTCCGACACTCTGGAGGTGATGCGCAAGCCCATGTGTGGAGTTCCTGACGT
TGGTCACTTCAGAACCTTTCCTGGCATCCCGAAGTGGAGGAAAACCCACCTTACATACAGGATTGTGA
ATTATACACCAGATTTGCCAAAAGATGCTGTTGATTCTGCTGTTGAGAAAGCTCTGAAAGTCTGGGAA
GAGGTGACTCCACTCACATTCTCCAGGCTGTATGAAGGAGAGACTGATATAATGATCTCTTTTGCAGT
TAGAGAACATGGAGACTTTTACCCTTTTGATGGACCTGGAAATGTTTTGGCCCATGCCTATGCCCCTG
GGCCAGGGATTAATGGAGATGCCCACTTTGATGATGATGAACAATGGACAAAGGATACAACAGGGACC
AATTTATTTCTCGTTGCTGCTCATGAAATTGGCCACTCCCTGGGTCTCTTTCACTCAGCCAACACTGA
AGCTTTGATGTACCCACTCTATCACTCACTCACAGACCTGACTCGGTTCCGCCTGTCTCAAGATGATA
TAAATGGCATTCAGTCCCTCTATGGACCTCCCCCTGACTCCCCTGAGACCCCCCTGGTACCCACGGAA
CCTGTCCCTCCAGAACCTGGGACGCCAGCCAACTGTGATCCTGCTTTGTCCTTTGATGCTGTCAGCAC
TCTGAGGGGAGAAATCCTGATCTTTAAAGACAGGCACTTTTGGCGCAAATCCCTCAGGAAGCTTGAAC
CTGAATTGCATTTGATCTCTTCATTTTGGCCATCTCTTCCTTCAGGCGTGGATGCCGCATATGAAGTT
ACTAGCAAGGACCTCGTTTTCATTTTTAAAGGAAATCAATTCTGGGCCATCAGAGGAAATGAGGTACG
AGCTGGATACCCAAGAGGCATCCACACCCTAGGTTTCCCTCCAACCGTGAGGAAAATCGATGCAGCCA
TTTCTGATAAGGAAAAGAACAAAACATATTTCTTTGTAGAGGACAAATACTGGAGATTTGATGAGAAG
AGAAATTCCATGGAGCCAGGCTTTCCCAAGCAAATAGCTGAAGACTTTCCAGGGATTGACTCAAAGAT
TGATGCTGTTTTTGAAGAATTTGGGTTCTTTTATTTCTTTACTGGATCTTCACAGTTGGAGTTTGACC
CAAATGCAAAGAAAGTGACACACACTTTGAAGAGTAACAGCTGGCTTAATTGTTGAAAGAGATATGTA
GAAGGCACAATATGGGCACTTTAAATGAAGCTAATAATTCTTCACCTAAGTCTCTGTGAATTGAAATG
TTCGTTTTCTCCTGCT

NOV75a, CG91708-02

| | | | |
|---|---|---|---|
| Protein Sequence | SEQ ID NO: 1082 | 477 aa | MW at 53982.7kD |

MKSLPILLLLCVAVCSAYPLDGAARGEDTSMNLVQKYLENYYDLEKDVKQFVRRKDSGPVVKKIREMQ
KFLGLEVTGKLDSDTLEVMRKPMCGVPDVGHFRTFPGIPKWRKTHLTYRIVNYTPDLPKDAVDSAVEK
ALKVWEEVTPLTFSRLYEGETDIMISFAVREHGDFYPFDGPGNVLAHAYAPGPGINGDAHFDDDEQWT
KDTTGTNLFLVAAHEIGHSLGLFHSARTEALMYPLYHSLTDLTRFRLSQDDINGIQSLYGPPPDSPET
PLVPTEPVPPEPGTPANCDPALSFDAVSTLRGEILIFKDRHFWRKSLRKLEPELHLISSFWPSLPSGV
DAAYEVTSKDLVFIFKGNQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFDPNAKKVTHTLKSNSWLN
C

| | | |
|---|---|---|
| NOV75b, 262751856 | SEQ ID NO: 1083 | 1446 bp |

| | | |
|---|---|---|
| DNA Sequence | ORF Start: at 1 | ORF Stop: end of sequence |

GGATCCACCATGAAGAGTCTTCCAATCCTACTGTTGCTGTGCGTGGCAGTTTGCTCAGCCTATCCATT
GGATGGAGCTGCAAGGGGTGAGGACACCAGCATGAACCTTGTTCAGAAATATCTAGAAAACTACTACG
ACCTCGAAAAAGATGTGAAACAGTTTGTTAGGAGAAAGGACAGTGGTCCTGTTGTTAAAAAAATCCGA
GAAATGCAGAAGTTCCTTGGATTGGAGGTGACGGGGAAGCTGGACTCCGACACTCTGGAGGTGATGCG
CAAGCCCAGGTGTGGAGTTCCTGACGTTGGTCACTTCAGAACCTTTCCTGGCATCCCGAAGTGGAGGA
AAACCCACCTTACATACAGGATTGTGAATTATACACCAGATTTGCCAAAAGATGCTGTTGATTCTGCT
GTTGAGAAAGCTCTGAAAGTCTGGGAAGAGGTGACTCCACTCACATTCTCCAGGCTGTATGAAGGAGA
GGCTGATATAATGATCTCTTTTGCAGTTAGAGAACATGGAGACTTTTACCCTTTTGATGGACCTGGAA
ATGTTTTGGCCCATGCCTATGCCCCTGGGCCAGGGATTAATGGAGATGCCCACTTTGATGATGATGAA
CAATGGACAAAGGATACAACAGGGACCAATTTATTTCTCGTTGCTGCTCATGAAATTGGCCACTCCCT
GGGTCTCTTTCACTCAGCCAACACTGAAGCTTTGATGTACCCACTCTATCACTCACTCACAGACCTGA
CTCGGTTCCGCCTGTCTCAAGATGATATAAATGGCATTCAGTCCCTCTATGGACCTCCCCCTGACTCC
CCTGAGACCCCCCTGGTACCCACGGAACCTGTCCCTCCAGAACCTGGGACGCCAGCCAACTGTGATCC
TGCTTTGTCCTTTGATGCTGTCAGCACTCTGAGGGGAGAAATCCTGATCTTTAAAGACAGGCACTTTT
GGCGCAAATCCCTCAGGAAGCTTGAACCTGAATTGCATTTGATCTCTTCATTTTGGCCATCTCTTCCT
TCAGGCGTGGATGCCGCATATGAAGTTACTAGCAAGGACCTCGTTTTCATTTTTAAAGGAAATCAATT
CTGGGCCATCAGAGGAAATGAGGTACGAGCTGGATACCCAAGAGGCATCCACACCCTAGGTTTCCCTC
CAACCGTGAGGAAAATCGATGCAGCCATTTCTGATAAGGAAAAGAACAAAACATATTTCTTTGTAGAG
GACAAATACTGGAGATTTGATGAGAAGAGAAATTCCATGGAGCCAGGCTTTCCCAAGCAAATAGCTGA
AGACTTTCCAGGGATTGACTCAAAGATTGATGCTGTTTTTGAAGAATTTGGGTTCTTTTATTTCTTTA
CTGGATCTTCACAGTTGGAGTTTGACCCAAATGCAAAGAAAGTGACACACACTTTGAAGAGTAACAGC
TGGCTTAATTGTCTCGAG

NOV75b, 262751856

| | | | |
|---|---|---|---|
| Protein Sequence | SEQ ID NO: 1084 | 482 aa | MW at 54465.2kD |

GSTMKSLPILLLLCVAVCSAYPLDGAARGEDTSMNLVQKYLENYYDLEKDVKQFVRRKDSGPVVKKIR
EMQKFLGLEVTGKLDSDTLEVMRKPRCGVPDVGHFRTFPGIPKWRKTHLTYRIVNYTPDLPKDAVDSA
VEKALKVWEEVTPLTFSRLYEGEADIMISFAVREHGDFYPFDGPGNVLAHAYAPGPGINGDAHFDDDE
QWTKDTTGTNLFLVAAHEIGHSLGLFHSANTEALMYPLYHSLTDLTRFRLSQDDINGIQSLYGPPPDS
PETPLVPTEPVPPEPGTPAMCDPALSFDAVSTLRGEILIFKDRHFWRKSLRKLEPELHLISSFWPSLP
SGVDAAYEVTSKDLVFIFKGNQFWAIRGNEVRAGYPRGIHTLGFPPTVRKIDAAISDKEKNKTYFFVE
DKYWRFDEKRNSMEPGFPKQIAEDFPGIDSKIDAVFEEFGFFYFFTGSSQLEFDPNAKKVTHTLKSNS
WLNCLE

| | | |
|---|---|---|
| NOV75c, CG91708-01 | SEQ ID NO: 1085 | 1821 bp |

| | | |
|---|---|---|
| DNA Sequence | ORF Start: ATG at 64 | ORF Stop: TGA at 1495 |

ACAAGGAGGCAGGCAAGACAGCAAGGCATAGAGACAACATAGAGCTAAGTAAAGCCAGTGGAAATGAA
GAGTCTTCCAATCCTACTGTTGCTGTGCGTGGCAGTTTGCTCAGCCTATCCATTGGATGGAGCTGCAA
GGGGTGAGGACACCAGCATGAACCTTGTTCAGAAATATCTAGAAAACTACTACGACCTCAAAAAAGAT
GTGAAACAGTTTGTTAGGAGAAAGGACAGTGGTCCTGTTGTTAAAAAAATCCGAGAAATGCAGAAGTT
CCTTGGATTGGAGGTGACGGGGAAGCTGGACTCCGACACTCTGGAGGTGATGCGCAAGCCCAGGTGTG
GAGTTCCTGATGTTGGTCACTTCAGAACCTTTCCTGGCATCCCGAAGTGGAGGAAAACCCACCTTACA
TACAGGATTGTGAATTATACACCAGATTTGCCAAAAGATGCTGTTGATTCTGCTGTTGAGAAAGCTCT
GAAAGTCTGGGAAGAGGTGACTCCACTCACATTCTCCAGGCTGTATGAAGGAGAGGCTGATATAATGA
TCTCTTTTGCAGTTAGAGAACATGGAGACTTTTACCCTTTTGATGGACCTGGAAATGTTTTGGCCCAT
GCCTATGCCCCTGGGCCAGGGATTAATGGAGATGCCCACTTTGATGATGATGAACAATGGACAAAGGA
TACAACAGGGACCAATTTATTTCTCGTTGCTGCTCATGAAATTGGCCACTCCCTGGGTCTCTTTCACT
CAGCCAACACTGAAGCTTTGATGTACCCACTCTATCACTCACTCACAGACCTGACTCGGTTCCGCCTG
TCTCAAGATGATATAAATGGCATTCAGTCCCTCTATGGACCTCCCCCTGACTCCCCTGAGACCCCCCT
GGTACCCACGGAACCTGTCCCTCCAGAACCTGGGACGCCAGCCAACTGTGATCCTGCTTTGTCCTTTG
ATGCTGTCAGCACTCTGAGGGGAGAAATCCTGATCTTTAAAGACAGGCACTTTTGGCGCAAATCCCTC
AGGAAGCTTGAACCTGAATTGCATTTGATCTCTTCATTTTGGCCATCTCTTCCTTCAGGCGTGGATGC
CGCATATGAAGTTACTAGCAAGGACCTCGTTTTCATTTTTAAAGGAAATCAATTCTGGGCCATCAGAG
GAAATGAGGTACGAGCTGGATACCCAAGAGGCATCCACACCCTAGGTTTCCCTCCAACCGTGAGGAAA
ATCGATGCAGCCATTTCTGATAAGGAAAAGAACAAAACATATrTCTTTGTAGAGGACAAATACTGGAG
ATTTGATGAGAAGAGAAATTCCATGGAGCCAGGCTTTCCCAAGCAAATAGCTGAAGACTTTCCAGGGA
TTGACTCAAAGATTGATGCTGTTTTTGAAGAATTTGGGTTCTTTTATTTCTTTACTGGATCTTCACAG
TTGGAGTTTGACCCAAATGCAAAGAAAGTGACACACACTTTGAAGAGTAACAGCTGGCTTAATTGTTG
AAAGAGATATGTAGAAGGCACAATATGGGCACTTTAAATGAAGCTAATAATTCTTCACCTAAGTCTCT
GTGAATTGAAATGTTCGTTTTCTCCTGCCTGTGCTGTGACTCGAGTCACACTCAAGGGAACTTGAGCG
TGAATCTGTATCTTGCCGGTCATTTTTATGTTATTACAGGGCATTCAAATGGGCTGCTGCTTAGCTTG
CACCTTGTCACATAGAGTGATCTTTCCCAAGAGAAGGGGAAGCACTCGTGTGCAACAGACAAGTGACT
GTATCTGTGTAGACTATTTGCTTATTTAATAAAGACGATTTGTCAGTTGTTTT

NOV75c, CG91708-01

| | | | |
|---|---|---|---|
| Protein Sequence | SEQ ID NO: 1086 | 477 aa | MW at 53976.7kD |

MKSLPILLLLCVAVCSAYPLDGAARGEDTSMNLVQKYLENYYDLEKDVKQFVRRKDSGPVVKKIREMQ
KFLGLEVTGKLDSDTLEVMRKPMCGVPDVGHFRTFPGIPKWRKTHLTYRIVNYTPDLPKDAVDSAVEK
ALKVWEEVTPLTFSRLYEGETDIMISFAVREHGDFYPFDGPGNVLAHAYAPGPGINGDAHFDDDEQWT
KDTTGTNLFLVAAHEIGHSLGLFHSARTEALMYPLYHSLTDLTRFRLSQDDINGIQSLYGPPPDSPET
PLVPTEPVPPEPGTPANCDPALSFDAVSTLRGEILIFKDRHFWRKSLRKLEPELHLISSFWPSLPSGV
DAAYEVTSKDLVFIFKGNQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFDPNAKKVTHTLKSNSWLN
C

| | | |
|---|---|---|
| NOV75d, CG91708-03 | SEQ ID NO: 1087 | 1446 bp |

| | | |
|---|---|---|
| DNA Sequence | ORF Start: ATG at 10 | ORF Stop: at 1441 |

GGATCCACCATGAAGAGTCTTCCAATCCTACTGTTGCTGTGCGTGGCAGTTTGCTCAGCCTATCCATT
GGATGGAGCTGCAAGGGGTGAGGACACCAGCATGAACCTTGTTCAGAAATATCTAGAAAACTACTACG
ACCTCGAAAAAGATGTGAAACAGTTTGTTAGGAGAAAGGACAGTGGTCCTGTTGTTAAAAAAATCCGA
GAAATGCAGAAGTTCCTTGGATTGGAGGTGACGGGGAAGCTGGACTCCGACACTCTGGAGGTGATGCG
CAAGCCCAGGTGTGGAGTTCCTGACGTTGGTCACTTCAGAACCTTTCCTGGCATCCCGAAGTGGAGGA
AAACCCACCTTACATACAGGATTGTGAATTATACACCAGATTTGCCAAAAGATGCTGTTGATTCTGCT
GTTGAGAAAGCTCTGAAAGTCTGGGAAGAGGTGACTCCACTCACATTCTCCAGGCTGTATGAAGGAGA
GGCTGATATAATGATCTCTTTTGCAGTTAGAGAACATGGAGACTTTTACCCTTTTGATGGACCTGGAA
ATGTTTTGGCCCATGCCTATGCCCCTGGGCCAGGGATTAATGGAGATGCCCACTTTGATGATGATGAA
CAATGGACAAAGGATACAACAGGGACCAATTTATTTCTCGTTGCTGCTCATGAAATTGGCCACTCCCT
GGGTCTCTTTCACTCAGCCAACACTGAAGCTTTGATGTACCCACTCTATCACTCACTCACAGACCTGA
CTCGGTTCCGCCTGTCTCAAGATGATATAAATGGCATTCAGTCCCTCTATGGACCTCCCCCTGACTCC
CCTGAGACCCCCCTGGTACCCACGGAACCTGTCCCTCCAGAACCTGGGACGCCAGCCAACTGTGATCC
TGCTTTGTCCTTTGATGCTGTCAGCACTCTGAGGGGAGAAATCCTGATCTTTAAAGACAGGCACTTTT
GGCGCAAATCCCTCAGGAAGCTTGAACCTGAATTGCATTTGATCTCTTCATTTTGGCCATCTCTTCCT
TCAGGCGTGGATGCCGCATATGAAGTTACTAGCAAGGACCTCGTTTTCATTTTTAAAGGAAATCAATT
CTGGGCCATCAGAGGAAATGAGGTACGAGCTGGATACCCAAGAGGCATCCACACCCTAGGTTTCCCTC
CAACCGTGAGGAAAATCGATGCAGCCATTTCTGATAAGGAAAAGAACAAAACATATTTCTTTGTAGAG
GACAAATACTGGAGATTTGATGAGAAGAGAAATTCCATGGAGCCAGGCTTTCCCAAGCAAATAGCTGA
AGACTTTCCAGGGATTGACTCAAAGATTGATGCTGTTTTTGAAGAATTTGGGTTCTTTTATTTCTTTA
CTGGATCTTCACAGTTGGAGTTTGACCCAAATGCAAAGAAAGTGACACACACTTTGAAGAGTAACAGC
TGGCTTAATTGTCTCGAG

NOV75d, CG91708-03

| | | | |
|---|---|---|---|
| Protein Sequence | SEQ ID NO: 1088 | 477 aa | MW at 53977.6kD |

MKSLPILLLLCVAVCSAYPLDGAARGEDTSMNLVQKYLENYYDLEKDVKQFVRRKDSGPVVKKIREMQ
KFLGLEVTGKLDSDTLEVMRKPRCGVPDVGHFRTFPGIPKWRKTHLTYRIVNYTPDLPKDAVDSAVEK
ALKVWEEVTPLTFSRLYEGEADIMISFAVREHGDFYPFDGPGNVLAHAYAPGPGINGDAHFDDDEQWT
KDTTGTNLFLVAAHEIGHSLGLFHSANTEALMYPLYHSLTDLTRFRLSQDDINGIQSLYGPPPDSPET
PLVPTEPVPPEPGTPANCDPALSFDAVSTLRGEILIFKDRHFWRKSLRKLEPELHLISSFWPSLPSGV
DAAYEVTSKDLVFIFKGNQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFDPNAKKVTHTLKSNSWLN
C

| | | |
|---|---|---|
| NOV75e, CG91708-04 | SEQ ID NO: 1089 | 1446 bp |

| | | |
|---|---|---|
| DNA Sequence | ORF Start: ATG at 10 | ORF Stop: at 1441 |

GGATCCACCATGAAGAGTCTTCCAATCCTACTGTTGCTGTGCGTGGCAGTTTGCTCAGCCTATCCATT
GGATGGAGCTGCAAGGGGTGAGGACACCAGCATGAACCTTGTTCAGAAATATCTAGAAAACTACTACG
ACCTCAAAAAAGATGTGAAACAGTTTGTTAGGAGAAAGGACAGTGGTCCTGTTGTTAAAAAAATCCGA
GAAATGCAGAAGTTCCTTGGATTGGAGGTGACGGGGAAGCTGGACTCCGACACTCTGGAGGTGATGCG
CAAGCCCAGGTGTGGAGTTCCTGACGTTGGTCACTTCAGAACCTTTCCTGGCATCCCGAAGTGGAGGA
AAACCCACCTTACATACAGGATTGTGAATTATACACCAGATTTGCCAAAAGATGCTGTTGATTCTGCT
GTTGAGAAAGCTCTGAAAGTCTGGGAAGAGGTGACTCCACTCACATTCTCCAGGCTGTATGAAGGAGA
GGCTGATATAATGATCTCTTTTGCAGTTAGAGAACATGGAGACTTTTACCCTTTTGATGGACCTGGAA
ATGTTTTGGCCCATGCCTATGCCCCTGGGCCAGGGATTAATGGAGATGCCCACTTTGATGATGATGAA
CAATGGACAAAGGATACAACAGGGACCAATTTATTTCTCGTTGCTGCTCATGAAATTGGCCACTCCCT
GGGTCTCTTTCACTCAGCCAACACTGAAGCTTTGATGTACCCACTCTATCACTCACTCACAGACCTGA
CTCGGTTCCGCCTGTCTCAAGATGATATAAATGGCATTCAGTCCCTCTATGGACCTCCCCCTGACTCC
CCTGAGACCCCCCTGGTACCCACGGAACCTGTCCCTCCAGAACCTGGGACGCCAGCCAACTGTGATCC
TGCTTTGTCCTTTGATGCTGTCAGCACTCTGAGGGGAGAAATCCTGATCTTTAAAGACAGGCACTTTT
GGCGCAAATCCCTCAGGAAGCTTGAACCTGAATTGCATTTGATCTCTTCATTCTGGCCATCTCTTCCT
TCAGGCGTGGATGCCGCATATGAAGTTACTAGCAAGGACCTCGTTTTCATTTTTAAAGGAAATCAATT
CTGGGCCATCAGAGGAAATGAGGTACGAGCTGGATACCCAAGAGGCATCCACACCCTAGGTTTCCCTC
CAACCGTGAGGAAAATCGATGCAGCCATTTCTGATAAGGAAAAGAACAAAACATATTTCTTTGTAGAG
GACAAATACTGGAGATTTGATGAGAAGAGAAATTCCATGGAGCCAGGCTTTCCCAAGCAAATAGCTGA
AGACTTTCCAGGGATTGACTCAAAGATTGATGCTGTTTTTGAAGAATTTGGGTTCTTTTATTTCTTTA
CTGGATCTTCACAGTTGGAGTTTGACCCAAATGCAAAGAAAGTGACACACACTTTGAAGAGTAACAGC
TGGCTTAATTGTCTCGAG

NOV75e, CG91708-04

| | | | |
|---|---|---|---|
| Protein Sequence | SEQ ID NO: 1090 | 477 aa | MW at 53976.7kD |

MKSLPILLLLCVAVCSAYPLDGAARGEDTSMNLVQKYLENYYDLKKDVKQFVRRKDSGPVVKKIREMQ
KFLGLEVTGKLDSDTLEVMRKPRCGVPDVGHFRTFPGIPKWRKTHLTYRIVNYTPDLPKDAVDSAVEK
ALKVWEEVTPLTFSRLYEGEADIMISFAVREHGDFYPFDGPGNVLAHAYAPGPGINGDAHFDDDEQWT
KDTTGTNLFLVAAHEIGHSLGLFHSANTEALMYPLYHSLTDLTRFRLSQDDINGIQSLYGPPPDSPET
PLVPTEPVPPEPGTPANCDPALSFDAVSTLRGEILIFKDRHFWRKSLRKLEPELHLISSFWPSLPSGV
DAAYEVTSKDLVFIFKGNQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFDPNAKKVTHTLKSNSWLN
C

| | |
|---|---|
| SEQ ID NO: 1091 | 1580 bp |

| | | |
|---|---|---|
| NOV75f, SNP13380740 of | ORF Start: ATG at 51 | ORF Stop: TGA at 1482 |

| | | |
|---|---|---|
| CG91708-02, DNA Sequence | SNP Pos: 183 | SNP Change: G to A |

CAAGACAGCAAGGCATAGAGACAACATAGAGCTAAGTAAAGCCAGTGGAAATGAAGAGTCTTCCAATC
CTACTGTTGCTGTGCGTGGCAGTTTGCTCAGCCTATCCATTGGATGGAGCTGCAAGGGGTGAGGACAC
CAGCATGAACCTTGTTCAGAAATATCTAGAAAACTACTACGACCTCAAAAAAGATGTGAAACAGTTTG
TTAGGAGAAAGGACAGTGGTCCTGTTGTTAAAAAAATCCGAGAAATGCAGAAGTTCCTTGGATTGGAG
GTGACGGGGAAGCTGGACTCCGACACTCTGGAGGTGATGCGCAAGCCCATGTGTGGAGTTCCTGACGT
TGGTCACTTCAGAACCTTTCCTGGCATCCCGAAGTGGAGGAAAACCCACCTTACATACAGGATTGTGA
ATTATACACCAGATTTGCCAAAAGATGCTGTTGATTCTGCTGTTGAGAAAGCTCTGAAAGTCTGGGAA
GAGGTGACTCCACTCACATTCTCCAGGCTGTATGAAGGAGAGACTGATATAATGATCTCTTTTGCAGT
TAGAGAACATGGAGACTTTTACCCTTTTGATGGACCTGGAAATGTTTTGGCCCATGCCTATGCCCCTG
GGCCAGGGATTAATGGAGATGCCCACTTTGATGATGATGAACAATGGACAAAGGATACAACAGGGACC
AATTTATTTCTCGTTGCTGCTCATGAAATTGGCCACTCCCTGGGTCTCTITCACTCAGCCAACACTGA
AGCTTTGATGTACCCACTCTATCACTCACTCACAGACCTGACTCGGTTCCGCCTGTCTCAAGATGATA
TAAATGGCATTCAGTCCCTCTATGGACCTCCCCCTGACTCCCCTGAGACCCCCCTGGTACCCACGGAA
CCTGTCCCTCCAGAACCTGGGACGCCAGCCAACTGTGATCCTGCTTTGTCCTTTGATGCTGTCAGCAC
TCTGAGGGGAGAAATCCTGATCTTTAAAGACAGGCACTTTTGGCGCAAATCCCTCAGGAAGCTTGAAC
CTGAATTGCATTTGATCTCTTCATTTTGGCCATCTCTTCCTTCAGGCGTGGATGCCGCATATGAAGTT
ACTAGCAAGGACCTCGTTTTCATTTTTAAAGGAAATCAATTCTGGGCCATCAGAGGAAATGAGGTACG
AGCTGGATACCCAAGAGGCATCCACACCCTAGGTTTCCCTCCAACCGTGAGGAAAATCGATGCAGCCA
TTTCTGATAAGGAAAAGAACAAAACATATTTCTTTGTAGAGGACAAATACTGGAGATTTGATGAGAAG
AGAAATTCCATGGAGCCAGGCTTTCCCAAGCAAATAGCTGAAGACTTTCCAGGGATTGACTCAAAGAT
TGATGCTGTTTTTGAAGAATTTGGGTTCTTTTATTTCTTTACTGGATCTTCACAGTTGGAGTTTGACC
CAAATGCAAAGAAAGTGACACACACTTTGAAGAGTAACAGCTGGCTTAATTGTTGAAAGAGATATGTA
GAAGGCACAATATGGGCACTTTAAATGAAGCTAATAATTCTTCACCTAAGTCTCTGTGAATTGAAATG
TTCGTTTTCTCCTGCT

| | | |
|---|---|---|
| NOV75f, SNP13380740 of | SEQ ID NO: 1092 | MW at 53981.8kD |

| | | | |
|---|---|---|---|
| CG91708-02, Protein Sequence | SNP Pos: 45 | 477 aa | SNP Change: Glu to Lys |

MKSLPILLLLCVAVCSAYPLDGAARGEDTSMNLVQKYLENYYDLKKDVKQFVRRKDSGPVVKKIREMQ
KFLGLEVTGKLDSDTLEVMRKPMCGVPDVGHFRTFPGIPKWRKTHLTYRIVNYTPDLPKDAVDSAVEK
ALKVWEEVTPLTFSRLYEGETDIMISFAVREHGDFYPFDGPGNVLAHAYAPGPGINGDAHFDDDEQWT
KDTTGTNLFLVAAHEIGHSLGLFHSANTEALMYPLYHSLTDLTRFRLSQDDINGIQSLYGPPPDSPET
PLVPTEPVPPEPGTPANCDPALSFDAVSTLRGEILIFKDRHFWRKSLRKLEPELHLISSFWPSLPSGV
DAAYEVTSKDLVFIFKGNQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFDPNAKKVTHTLKSNSWLN
C

A ClustalW comparison of the above protein sequences yields the following sequence alignment shown in Table 75B.

TABLE 75B
Comparison of the NOV75 protein sequences.

| | |
|---|---|
| NOV75a | ---MKSLPILLLLCVAVCSAYPLDGAARGEDTSMNLVQKYLENYYDLEKDVKQFVRRKDS |
| NOV75b | GSTMKSLPILLLLCVAVCSAYPLDGAARGEDTSMNLVQKYLENYYDLEKDVKQFVRRKDS |
| NOV75c | ---MKSLPILLLLCVAVCSAYPLDGAARGEDTSMNLVQKYLENYYDLKKDVKQFVRRKDS |
| NOV75d | ---MKSLPILLLLCVAVCSAYPLDGAARGEDTSMNLVQKYLENYYDLEKDVKQFVRRKDS |
| NOV75e | ---MKSLPILLLLCVAVCSAYPLDGAARGEDTSMNLVQKYLENYYDLKKDVKQFVRRKDS |
| NOV75a | GPVVKKIREMQKFLGLEVTGKLDSDTLEVMRKPMCGVPDVGHFRTFPGIPKWRKTHLTYR |
| NOV75b | GPVVKKIREMQKFLGLEVTGKLDSDTLEVMRKPRCGVPDVGHFRTFPGIPKWRKTHLTYR |
| NOV75c | GPVVKKIREMQKFLGLEVTGKLDSDTLEVMRKPRCGVPDVGHFRTFPGIPKWRKTHLTYR |
| NOV75d | GPVVKKIREMQKFLGLEVTGKLDSDTLEVMRKPRCGVPDVGHFRTFPGIPKWRKTHLTYR |
| NOV75e | GPVVKKIREMQKFLGLEVTGKLDSDTLEVMRKPRCGVPDVGHFRTFPGIPKWRKTHLTYR |
| NOV75a | IVNYTPDLPKDAVDSAVEKALKVWEEVTPLTFSRLYEGETDIMISFAVREHGDFYPFDGP |
| NOV75b | IVNYTPDLPKDAVDSAVEKALKVWEEVTPLTFSRLYEGEADIMISFAVREHGDFYPFDGP |
| NOV75c | IVNYTPDLPKDAVDSAVEKALKVWEEVTPLTFSRLYEGEADIMISFAVREHGDFYPFDGP |
| NOV75d | IVNYTPDLPKDAVDSAVEKALKVWEEVTPLTFSRLYEGEADIMISFAVREHGDFYPFDGP |
| NOV75e | IVNYTPDLPKDAVDSAVEKALKVWEEVTPLTFSRLYEGEADIMISFAVREHGDFYPFDGP |
| NOV75a | GNVLAHAYAPGPGINGDAHFDDDEQWTKDTTGTNLFLVAAHEIGHSLGLFHSANTEALMY |
| NOV75b | GNVLAHAYAPGPGINGDAHFDDDEQWTKDTTGTNLFLVAAHEIGHSLGLFHSANTEALMY |
| NOV75c | GNVLAHAYAPGPGINGDAHFDDDEQWTKDTTGTNLFLVAAHEIGHSLGLFHSANTEALMY |
| NOV75d | GNVLAHAYAPGPGINGDAHFDDDEQWTKDTTGTNLFLVAAHEIGHSLGLFHSANTEALMY |
| NOV75e | GNVLAHAYAPGPGINGDAHFDDDEQWTKDTTGTNLFLVAAHEIGHSLGLFHSANTEALMY |
| NOV75a | PLYHSLTDLTRFRLSQDDINGIQSLYGPPPDSPETPLVPTEPVPPEPGTPANCDPALSFD |
| NOV75b | PLYHSLTDLTRFRLSQDDINGIQSLYGPPPDSPETPLVPTEPVPPEPGTPANCDPALSFD |
| NOV75c | PLYHSLTDLTRFRLSQDDINGIQSLYGPPPDSPETPLVPTEPVPPEPGTPANCDPALSFD |
| NOV75d | PLYHSLTDLTRFRLSQDDINGIQSLYGPPPDSPETPLVPTEPVPPEPGTPANCDPALSFD |
| NOV75e | PLYHSLTDLTRFRLSQDDINGIQSLYGPPPDSPETPLVPTEPVPPEPGTPANCDPALSFD |
| NOV75a | AVSTLRGEILIFKDRHFWRKSLRKLEPELHLISSFWPSLPSGVDAAYEVTSKDLVFIFKG |
| NOV75b | AVSTLRGEILIFKDRHFWRKSLRKLEPELHLISSFWPSLPSGVDAAYEVTSKDLVFIFKG |
| NOV75c | AVSTLRGEILIFKDRHFWRKSLRKLEPELHLISSFWPSLPSGVDAAYEVTSKDLVFIFKG |
| NOV75d | AVSTLRGEILIFKDRHFWRKSLRKLEPELHLISSFWPSLPSGVDAAYEVTSKDLVFIFKG |
| NOV75e | AVSTLRGEILIFKDRHFWRKSLRKLEPELHLISSFWPSLPSGVDAAYEVTSKDLVFIFKG |
| NOV75a | NQFWAIRGNEVRAGYPRGIHTLGFPPTVRKIDAAISDKEKNKTYFFVEDKYWRFDEKRNS |
| NOV75b | NQFWAIRGNEVRAGYPRGIHTLGFPPTVRKIDAAISDKEKNKTYFFVEDKYWRFDEKRNS |
| NOV75c | NQFWAIRGNEVRAGYPRGIHTLGFPPTVRKIDAAISDKEKNKTYFFVEDKYWRFDEKRNS |
| NOV75d | NQFWAIRGNEVRAGYPRGIHTLGFPPTVRKIDAAISDKEKNKTYFFVEDKYWRFDEKRNS |
| NOV75e | NQFWAIRGNEVRAGYPRGIHTLGFPPTVRKIDAAISDKEKNKTYFFVEDKYWRFDEKRNS |
| NOV75a | MEPGFPKQIAEDFPGIDSKIDAVFEEFGFFYFFTGSSQLEFDPNAKKVTHTLKSNSWLNC |
| NOV75b | MEPGFPKQIAEDFPGIDSKIDAVFEEFGFFYFFTGSSQLEFDPNAKKVTHTLKSNSWLNC |
| NOV75c | MEPGFPKQIAEDFPGIDSKIDAVFEEFGFFYFFTGSSQLEFDPNAKKVTHTLKSNSWLNC |
| NOV75d | MEPGFPKQIAEDFPGIDSKIDAVFEEFGFFYFFTGSSQLEFDPNAKKVTHTLKSNSWLNC |
| NOV75e | MEPGFPKQIAEDFPGIDSKIDAVFEEFGFFYFFTGSSQLEFDPNAKKVTHTLKSNSWLNC |
| NOV75a | -- |
| NOV75b | LE |
| NOV75c | -- |
| NOV75d | -- |
| NOV75e | -- |
| NOV75a | (SEQ ID NO: 1082) |
| NOV75b | (SEQ ID NO: 1084) |
| NOV75c | (SEQ ID NO: 1086) |
| NOV75d | (SEQ ID NO: 1088) |
| NOV75e | (SEQ ID NO: 1090) |

Further analysis of the NOV75a protein yielded the following properties shown in Table 75C.

TABLE 75C
Protein Sequence Properties NOV75a

| | |
|---|---|
| SignalP analysis: | Cleavage site between residues 18 and 19 |

PSORT II analysis:

| PSG: | a new signal peptide prediction method | | | |
|---|---|---|---|---|
| | N-region: | length 2; | pos.chg 1; | neg.chg 0 |

| | |
|---|---|
| H-region: | length 18; peak value 10.73 |

| | PSG score: 6.33 |
|---|---|
| GvH: | von Heijne's method for signal seq. recognition |
| | GvH score (threshold: −2.1): 3.45 |
| | possible cleavage site: between 17 and 18 |

>>> Seems to have a cleavable signal peptide (1 to 17)

| | |
|---|---|
| ALOM: | Klein et al's method for TM region allocation |
| | Init position for calculation: 18 |
| | Tentative number of TMS(s) for the threshold 0.5: 0 |
| | number of TMS(s) . . . fixed |
| | PERIPHERAL Likelihood = 3.87 (at 293) |
| | ALOM score: 3.87 (number of TMSs: 0) |
| MTOP: | Prediction of membrane topology (Hartmann et al.) |
| | Center position for calculation: 8 |
| | Charge difference: −4.0 C(−2.0) − N(2.0) |
| | N >= C: N-terminal side will be inside |

MITDISC: discrimination of mitochondrial targeting seq

| | | | |
|---|---|---|---|
| R content: | 0 | Hyd Moment (75): | 3.04 |
| Hyd Moment (95): | 6.62 | G content: | 0 |
| D/E content: | 1 | S/T content: | 2 |
| Score: −4.76 | | | |

Gavel: prediction of cleavage sites for mitochondrial preseq

cleavage site motif not found

NUCDISC: discrimination of nuclear localization signals

pat4: none
pat7: PVVKKIR (3) at 59
pat7: PKWRKTH (5) at 107
bipartite: none
content of basic residues: 11.3%
NLS Score: 0.22

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

```
  33.3%: extracellular, including cell wall
  22.2%: mitochondrial
  22.2%: endoplasmic reticulum
  11.1%: Golgi
  11.1%: vacuolar
```

>> prediction for CG91708-02 is exc (k = 9)

A search of the NOV75a protein against the Geneseq database, a proprietary database that contains sequences published in patents and patent publication, yielded several homologous proteins shown in Table 75D.

TABLE 75D
Geneseq Results for NOV75a

| | | NOV75a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB04752 | Human MMP3 protein SEQ ID | 1 . . . 477 | 475/477 (99%) | 0.0 |
| | NO: 3 - Homo sapiens, 477 aa. | 1 . . . 477 | 475/477 (99%) | |
| | [WO200179238-A2, 25-OCT- | | | |
| | 2001] | | | |
| AAB84608 | Amino acid sequence of matrix | 1 . . . 477 | 475/477 (99%) | 0.0 |
| | metalloproteinase-3 stromelysin 1 - | 1 . . . 477 | 475/477 (99%) | |
| | Homo sapiens, 477 aa. | | | |
| | [WO200149309-A2, 12-JUL- | | | |
| | 2001] | | | |
| AAO20482 | Prostromelysin protein - Homo | 1 . . . 477 | 475/477 (99%) | 0.0 |
| | sapiens, 477 aa. [US6284513-B1, | 1 . . . 477 | 475/477 (99%) | |
| | 04-SEP-2001] | | | |
| ABP54461 | Matrix metalloproteinase 3 amino | 1 . . . 477 | 474/477 (99%) | 0.0 |
| | acid sequence SEQ ID NO: 2919 - | 1 . . . 477 | 475/477 (99%) | |
| | Homo sapiens, 477 aa. | | | |
| | [WO200278516-A2, 10-OCT- | | | |
| | 2002] | | | |
| ABU03473 | Angiogenesis-associated human | 1 . . . 477 | 474/477 (99%) | 0.0 |
| | protein sequence #18 - Homo | 1 . . . 477 | 475/477 (99%) | |
| | sapiens, 477 aa. [WO200279492- | | | |
| | A2, 10-OCT-2002] | | | |

In a BLAST search of public sequence databases, the NOV75a protein was found to have homology to the proteins shown in the BLASTP data in Table 75E.

TABLE 75E
Public BLASTP Results for NOV75a

| | | NOV75a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P08254 | Stromelysin-1 precursor (EC | 1 . . . 477 | 474/477 (99%) | 0.0 |
| | 3.4.24.17) (Matrix | 1 . . . 477 | 475/477 (99%) | |
| | metalloproteinase-3) (MMP-3) | | | |
| | (Transin-1) (SL-1) - Homo sapiens | | | |
| | (Human), 477 aa. | | | |
| P28863 | Stromelysin-1 precursor (EC | 1 . . . 477 | 401/478 (83%) | 0.0 |
| | 3.4.24.17) (Matrix | 1 . . . 478 | 433/478 (89%) | |
| | metalloproteinase-3) (MMP-3) | | | |
| | (Transin-1) (SL-1) - Oryctolagus | | | |
| | cuniculus (Rabbit), 478 aa. | | | |
| Q28397 | Stromelysin-1 precursor (EC | 1 . . . 477 | 386/477 (80%) | 0.0 |
| | 3.4.24.17) (Matrix | 1 . . . 477 | 427/477 (88%) | |
| | metalloproteinase-3) (MMP-3) - | | | |
| | Equus caballus (Horse), 477 aa. | | | |
| P09238 | Stromelysin-2 precursor (EC | 1 . . . 477 | 372/477 (77%) | 0.0 |
| | 3.4.24.22) (Matrix | 1 . . . 476 | 418/477 (86%) | |
| | metalloproteinase-10) (MMP-10) | | | |
| | (Transin-2) (SL-2) - Homo sapiens | | | |
| | (Human), 476 aa. | | | |
| Q922W6 | Matrix metalloproteinase 3 - Mus | 1 . . . 477 | 366/477 (76%) | 0.0 |
| | musculus (Mouse), 479 aa. | 3 . . . 479 | 413/477 (85%) | |

PFam analysis indicates that the NOV75a protein contains the domains shown in the Table 75F.

TABLE 75F
Domain Analysis of NOV75a

| | NOV75a | Identities/Similarities | Expect |
|---|---|---|---|
| Pfam Domain | Match Region | for the Matched Region | Value |
| Peptidase_M10_N | 18 . . . 96 | 44/84 (52%) | 1.2e−42 |
| | | 75/84 (89%) | |
| Peptidase_M10 | 102 . . . 208 | 78/107 (73%) | 1.6e−80 |
| | | 105/107 (98%) | |
| Astacin | 107 . . . 267 | 37/230 (16%) | 0.17 |
| | | 106/230 (46%) | |
| hemopexin | 296 . . . 338 | 16/50 (32%) | 5.1e−12 |
| | | 37/50 (74%) | |
| hemopexin | 340 . . . 383 | 16/50 (32%) | 5.6e−13 |
| | | 39/50 (78%) | |
| hemopexin | 388 . . . 435 | 25/50 (50%) | 6.6e−19 |
| | | 41/50 (82%) | |
| hemopexin | 437 . . . 477 | 17/50 (34%) | 1.5e−09 |
| | | 33/50 (66%) | |

### Example 76

The NOV76 clone was analyzed, and the nucleotide and encoded polypeptide sequences are shown in Table 76A.

TABLE 76A
NOV76 Sequence Analysis

| | | |
|---|---|---|
| NOV76a, CG92078-02 | SEQ ID NO: 1093 | 1983 bp |

| | | |
|---|---|---|
| DNA Sequence | ORF Start: ATG at 6 | ORF Stop: TGA at 1836 |

GCAGCATGAGCCGATCACCCCTCAATCCCAGCCAACTCCGATCAGTGGGCTCCCAGGATGCCCTGGCC
CCCTTGCCTCCACCTGCTCCCCAGAATCCCTCCACCCACTCTTGGGACCCTTTGTGTGGATCTCTGCC
TTGGGGCCTCAGCTGTCTTCTGGCTCTGCAGCATGTCTTGGTCATGGCTTCTCTGCTCTGTGTCTCCC
ACCTGCTCCTGCTTTGCAGTCTCTCCCCAGGAGGACTCTCTTACTCCCCTTCTCAGCTCCTGGCCTCC
AGCTTCTTTTCATGTGGTATGTCTACCATCCTGCAAACTTGGATGGGCAGCAGGCTGCCTCTTGTCCA
GGCTCCATCCTTAGAGTTCCTTATCCCTGCTCTGGTGCTGACCAGCCAGAAGCTACCCCGGGCCATCC
AGACACCTGGAAACTCCTCCCTCATGCTGCACCTTTGTAGGGGACCTAGCTGCCATGGCCTGGGGCAC
TGGAACACTTCTCTCCAGGAGGTGTCCGGGGCAGTGGTAGTATCTGGGCTGCTGCAGGGCATGATGGG
GCTGCTGGGGAGTCCCGGCCACGTGTTCCCCCACTGTGGGCCCCTGGTGCTGGCTCCCAGCCTGGTTG
TGGCAGGGCTCTCTGCCCACAGGGAGGTAGCCCAGTTCTGCTTCACACACTGGGGGTTGGCCTTGCTG
GTTATCCTGCTCATGGTGGTCTGTTCTCAGCACCTGGGCTCCTGCCAGTTTCATGTGTGCCCCTGGAG
GCGAGCTTCAACGTCATCAACTCACACTCCTCTCCCTGTCTTCCGGCTCCTTTCGGTGCTGATCCCAG
TGGCCTGTGTGTGGATTGTTTCTGCCTTTGTGGGATTCAGTGTTATCCCCCAGGAACTGTCTGCCCCC
ACCAAGGCACCATGGATTTGGCTGCCTCACCCAGGTGAGTGGAATTGGCCTTTGCTGACGCCCAGAGC
TCTGGCTGCAGGCATCTCCATGGCCTTGGCAGCCTCCACCAGTTCCCTGGGCTGCTATGCCCTGTGTG
GCCGGCTGCTGCATTTGCCTCCCCCACCTCCACATGCCTGCAGTCGAGGGCTGAGCCTGGAGGGGCTG
GGCAGTGTGCTGGCCGGGCTGCTGGGAAGCCCCATGGGCACTGCATCCAGCTTCCCCAGCGTGGGCAA
AGTGGGTCTTATCCAGGCTGGATCTCAGCAAGTGGCTCACTTAGTGGGGCTACTCTGCGTGGGGCTTG
GACTCTCCCCCAGGTTGGCTCAGCTCCTCACCACCATCCCACTGCCTGTTGTTGGTGGGGTGCTGGGC
GTGACCCAGGCTGTGGTTTTGTCTGCTGGATTCTCCAGCTTCTACCTGGCTGACATAGACTCTGGGCG
AAATATCTTCATTGTGGGCTTCTCCATCTTCATGGCCTTGCTGCTGCCAAGATGGTTTCGGGAAGCCC
CAGTCCTGTTCAGCACAGGCTGGAGCCCCTTGGATGTATTACTGCACTCACTGCTGACACAGCCCATC
TTCCTGGCTGGACTCTCAGGCTTCCTACTAGAGAACACGATTCCTGGCACACAGCTTGAGCGAGGCCT
AGGTCAAGGGCTACCATCTCCTTTCACTGCCCAAGAGGCTCGAATGCCTCAGAAGCCCAGGGAGAAGG
CTGCTCAAGTGTACAGACTTCCTTTCCCCATCCAAAACCTCTGTCCCTGCATCCCCCAGCCTCTCCAC
TGCCTCTGCCCACTGCCTGAAGACCCTGGGGATGAGGAAGGAGGCTCCTCTGAGCCAGAAGAGATGGC
AGACTTGCTGCCTGGCTCAGGGGAGCCATGCCCTGAATCTAGCAGAGAAGGGTTTAGGTCCCAGAAAT
GACCAGAACGCCTACTTCTGCCCTGGTTAATTTAGCCCTAACTCTCATCTGCTGGAGAGTCAGCTCCC
AAACTGTTCTTTCTTGTAGGCAGAGGATATGTGTGTGTGTATTACATGGGACTGTCTAGAGGTTCCAT
TTCCCAATAGG

NOV76a, CG92078-02

| | | | |
|---|---|---|---|
| Protein Sequence | SEQ ID NO: 1094 | 610 aa | MW at 64502.7kD |

MSRSPLNPSQLRSVGSQDALAPLPPPAPQNPSTHSWDPLCGSLPWGLSCLLALQHVLVMASLLCVSHL
LLLCSLSPGGLSYSPSQLLASSFFSCGMSTILQTWNGSRLPLVQAPSLEFLIPALVLTSQKLPRAIQT
PGNSSLMLHLCRGPSCHGLGHWNTSLQEVSGAVVVSGLLQGMMGLLGSPGHVFPHCGPLVLAPSLVVA
GLSAHREVAQFCFTHWGLALLVILLMVVCSQHLGSCQFHVCPWRRASTSSTHTPLPVFRLLSVLIPVA
CVWIVSAFVGFSVIPQELSAPTKAPWIWLPHPGEWNWPLLTPRALAAGISMALAASTSSLGCYALCGR
LLHLPPPPPHACSRGLSLEGLGSVLAGLLGSPMGTASSFPSVGKVGLIQAGSQQVAHLVGLLCVGLGL
SPRLAQLLTTIPLPVVGGVLGVTQAVVLSAGFSSFYLADIDSGRNIFIVGFSIFMALLLPRWFREAPV
LFSTGWSPLDVLLHSLLTQPIFLAGLSGFLLENTIPGTQLERGLGQGLPSPFTAQEARMPQKPREKAA
QVYRLPFPIQNLCPCIPQPLHCLCPLPEDPGDEEGGSSEPEEMADLLPGSGEPCPESSREGFRSQK

| | | |
|---|---|---|
| NOV76b, CG92078-01 | SEQ ID NO: 1095 | 2014 bp |

| | | |
|---|---|---|
| DNA Sequence | ORF Start: ATG at 6 | ORF Stop: TAG at 1992 |

GCAGCATGAGCCGATCACCCCTCAATCCCAGCCAACTCCGATCAGTGGGCTCCCAGGATGCCCTGGCC
CCCTTGCCTCCACCTGCTCCCCAGAATCCCTCCACCCACTCTTGGGACCCTTTGTGTGGATCTCTGCC
TTGGGGCCTCAGCTGTCTTCTGGCTCTGCAGCATGTCTTGGTCATGGCTTCTCTGCTCTGTGTCTCCC
ACCTGCTCCTGCTTTGCAGTCTCTCCCCAGGAGGACTCTCTTACTCCCCTTCTCAGCTCCTGGCCTCC
AGCTTCTTTTCATGTGGTATGTCTACCATCCTGCAAACTTGGATGGGCAGCAGGAGGCTGCCTCTTGT
CCAGGCTCCATCCTTAGAGTTCCTTATCCCTGCTCTGGTGCTGACCAGCCAGAAGCTACCCCGGGCCA
TCCAGACACCTGGAAACGCCTCCCTCATGCTGCACCTTTGTAGGGGACCTAGCTGCCATGGCCTGGGG
CACTGGAACACTTCTCTCCAGGAGGTGGTGGTAGTATCTGGGCTGCTGCAGGGCATGATGGGGCTGCT
GGGGAGTCCCGGCCACGTGTTCCCCCACTGTGGGCCCCTGGTGCTGGCTCCCAGCCTGGTTGTGGCAG
GGCTCTCTGCCTTTCCCCAAGAGGGAGTTTTGCTCCTGTCACCCAGCCTGGAGTGCAATGGCATGATC
TCGGCTCACCACTGGGGAGAAGCCAGCCAGCTCTGCCTCCTGGCCCTGGAGTGCTCACTCCATCCCCT
ACCTTTTGGCTTCTGTCTACCCCTGCAAGGCTGGCTCAGAAGGTTCTGGGGGAGGAGTTCTTTTCTCA
GTCTCGCCCCTCAGGTGCTGATCCCAGTGGCCTGTGTGTGGATTGTTTCTGCCTTTGTGGGATTCAGT
GTTATCCCCCAGGAACTGTCTGCCCCCACCAAGGCACCATGGATTTGGCTGCCTCACCCAGGTGTGTG
GAATTGGCCTTTGCTGACGCCCAGAGCTCTGGCTGCAGGCATCTCCATGGCCTTGGCAGCCTCCACCA
GTTCCCTGGGCTGCTATGCCCTGTGTGGCCGGCTGCTGCATTTGCCTCCCCCACCTCCACATGCCTGC
AGTCGAGGGCTGAGCCTGGAGGGGCTGGGCAGTGTGCTGGCCGGGCTGCTGGGAAGCCCCATGGGCAC
TGCATCCAGCTTCCCCAACGTGGGCAAAGTGGGTCTTATCCAGCAGGCTGGATCTCAGCAAGTGGCTC
ACTTAGTGGGGCTACTCTGCGTGGGGCTTGGACTCTCCCCCAGGTTGGCTCAGCTCCTCACCACCATC
CCACTGCCTGTTGTTGGTGGTGGGGTGCTGGGGGTGACCCAGGCTGTGGTTTTGTCTGCTGGATTCTC
CAGCTTCTACCTGGCTGACATAGACTCTGGGCGAAATATCTTCATTGTGGGCTTCTCCATCTTCATGG
CCTTGCTGCTGCCAAGATGGTTTCGGGAAGCCCCAGTCCTGTTCAGCACAGGTCACTCACTGCTGATG
GAGCCCCTTGGATGTATTACTGCACAGCCCATCTTCCTGGCTGGACTCTCAGGCTTCCTACTAGAGAA
CACGATTCGGGGCACACAGCTTGAGCGAGGCCTAGGTCAAGGGCTACCATCTCCTTTCACTGCCCAAG
AGGCTCGAATGCCTCAGAAGCCCAGGGAGAAGGCTGCTCAAGTGTACAGACTTCCTTTCCCCATCCAA
AACCTCTGTCCCTGCATCCCCCAGCCTCTCCACTGCCTCTGCCCACTGCCTGAAGACCCTGGGGATGA
GGAAGGAGGCTCCTCTGAGCAGCAAGAGATGGCAGACTTGCTGCGTGGCTCAGGGGAGCATGCCCTGA
ATCTAGCAGAGAAGGGTTTAGGTCCAGAAATGACCAGAACGCGTACTTCTGCCCTGGTTAATTTAGCC
CTAACTCTCATCTGCTGGAGAGTCAGCTCCCAAACTGTTCTTTCTTGTAGGCAGAGGATATGTGTGTG
TGTATTACATGGGACTGTCTAGAGGTTCCATTTCCCAATAGG

NOV76b, CG92078-01

| | | | |
|---|---|---|---|
| Protein Sequence | SEQ ID NO: 1096 | 662 aa | MW at 70138.5kD |

MSRSPLNPSQLRSVGSQDALAPLPPPAPQNPSTHSWDPLCGSLPWGLSCLLALQHVLVMASLLCVSHL
LLLCSLSPGGLSYSPSQLLASSFFSCGMSTILQTWMGSRRLPLVQAPSLEFLIPALVLTSQKLPRAIQ
TPGNASLMLHLCRGPSCHGLGHWNTSLQEVVVVSGLLQGMMGLLGSPGHVFPHCGPLVLAPSLVVAGL
SAFPQEGVLLLSPSLECNGMISAHHWGEASQLCLLALECSLHPLPFGFCLPLQGWLRRFWGRSSFLSL
APQVLIPVACVWIVSAFVGFSVIPQELSAPTKAPWIWLPHPGVWNWPLLTPRALAAGISMAIAASTSS
LGCYALCGRLLHLPPPPPHACSRGLSLEGLGSVLAGLLGSPMGTASSFPNVGKVGLIQQAGSQQVAHL
VGLLCVGLGLSPRLAQLLTTIPLPVVGGGVLGVTQAVVLSAGFSSFYLADIDSGRNIFlVGFSIFMAL
LLPRWFREAPVLFSTGHSLLMEPLGCITAQPIFLAGLSGFLLENTIRGTQLERGLGQGLPSPFTAQEA
RMPQKPREKAAQVYRLPFPIQNLCPCIPQPLHCLCPLPEDPGDEEGGSSEQQEMADLLRGSGEHALNL
AEKGLGPEMTRTRTSALVNLALTLICWRVSSQTVLSCRQRICVCVLHGTV

A ClustalW comparison of the above protein sequences yields the following sequence alignment shown in Table 76B.

TABLE 76B
Comparison of the NOV76 protein sequences.

| | |
|---|---|
| NOV76a | MSRSPLNPSQLRSVGSQDALAPLPPPAPQNPSTHSWDPLCGSLPWGLSCLLALQHVLVMA |
| NOV76b | MSRSPLNPSQLRSVGSQDALAPLPPPAPQNPSTHSWDPLCGSLPWGLSCLLALQHVLVMA |
| NOV76a | SLLCVSHLLLLCSLSPGGLSYSPSQLLASSFFSCGMSTILQTWMGSRRLPLVQAPSLEFL |
| NOV76b | SLLCVSHLLLLCSLSPGGLSYSPSQLLASSFFSCGMSTILQTWMGSRRLPLVQAPSLEFL |
| NOV76a | IPALVLTSQKLPRAIQTPGNSSLMLHLCRGPSCHGLGNWNTSLQEVSGAVVVSGLLQGMM |
| NOV76b | IPALVLTSQKLPRAIQTPGNASLMLHLCRGPSCHGLGHWNTSLQEV---VVVSGLLQGMM |
| NOV76a | GLLGSPGHVFPHCGPLVLAPSLVVAGLSAHREVAQFCFT------------HWGLALLVI |
| NOV76b | GLLGSPGHVFPHCGPLVLAPSLVVAGLSAFPQEGVLLLSPSLECNGMISAHHWGEASQLC |
| NOV76a | LLMVVCSQH---LGSCQFHVCPWRRASTSSTHTPLPVFRLLSVLIPVACVWIVSAFVGFS |
| NOV76b | LLALECSLHPLPFGFCLPLQGWLRRFWGRSSFLSLAP----QVLIPVACVWIVSAFVGFS |
| NOV76a | VIPQELSAPTKAPWIWLPHPGEWNWPLLTPRALAAGISMALAASTSSLGCYALCGRLLHL |
| NOV76b | VIPQELSAPTKAPWIWLPHPGVWNWPLLTPRALAAGISMALAASTSSLGCYALCGRLLHL |
| NOV76a | PPPPPHACSRGLSLEGLGSVLAGLLGSPMGTASSFPSVGKVGLIQAG-SQQVAHLVGLLC |
| NOV76b | PPPPPHACSRGLSLEGLGSVLAGLLGSPMGTASSFPNVGKVGLIQQAGSQQVAHLVGLLC |
| NOV76a | VGLGLSPRLAQLLTTIPLPVVGG-VLGVTQAVVLSAGFSSFYLADIDSGRNIFIVGFSIF |
| NOV76b | VGLGLSPRLAQLLTTIPLPVVGGGVLGVTQAVVLSAGFSSFYLADIDSGRNIFIVGFSIF |
| NOV76a | MALLLPRWFREAPVLFSTGWSPLDVLLHSLLTQPIFLAGLSGFLLENTIPGTQLERGLGQ |
| NOV76b | MALLLPRWFREAPVLFSTGHSLLMEPLGCITAQPIFLAGLSGFLLENTIRGTQLERGLGQ |
| NOV76a | GLPSPFTAQEARMPQKPREKAAQVYRLPFPIQNLCPCIPQPLHCLCPLPEDPGDEEGGSS |
| NOV76b | GLPSPFTAQEARMPQKPREKAAQVYRLPFPIQNLCPCIPQPLHCLCPLPEDPGDEEGGSS |
| NOV76a | EPEEMADLLPGSGEPCPESSREGFRSQK-------------------------------- |
| NOV76b | EQQEMADLLRGSGEHALNLAEKGLGPEMTRTRTSALVNLALTLICWRVSSQTVLSCRQRI |
| NOV76a | --------- |
| NOV76b | CVCVLHGTV |
| NOV76a | (SEQ ID NO: 1094) |
| NOV76b | (SEQ ID NO: 1096) |

Further analysis of the NOV76a protein yielded the following properties shown in Table 76C.

TABLE 76C
Protein Sequence Properties NOV76a

| | |
|---|---|
| SignalP analysis: | Cleavage site between residues 17 and 18 |

PSORT II analysis:

| | |
|---|---|
| PSG: | a new signal peptide prediction method |
| | N-region: length 3; pos.chg 1; neg.chg 0 |
| | H-region: length 8; peak value −1.31 |
| | PSG score: −5.71 |
| GvH: | von Heijne's method for signal seq. recognition |
| | GvH score (threshold: −2.1): −4.74 |
| | possible cleavage site: between 48 and 49 |

>>> Seems to have no N-terminal signal peptide.

| ALOM: | Klein et al's method for TM region allocation | | |
|---|---|---|---|
| | Init position for calculation: 1 | | |
| | Tentative number of TMS(s) for the threshold 0.5: 9 | | |
| | INTEGRAL | Likelihood = −8.60 | Transmembrane 56-72 |
| | INTEGRAL | Likelihood = 0.16 | Transmembrane 110-126 |
| | INTEGRAL | Likelihood = −0.11 | Transmembrane 167-183 |
| | INTEGRAL | Likelihood = −1.70 | Transmembrane 192-208 |
| | INTEGRAL | Likelihood = −6.90 | Transmembrane 216-232 |
| | INTEGRAL | Likelihood = −8.23 | Transmembrane 265-281 |
| | INTEGRAL | Likelihood = −1.97 | Transmembrane 421-437 |
| | INTEGRAL | Likelihood = −3.24 | Transmembrane 454-470 |
| | INTEGRAL | Likelihood = 0.21 | Transmembrane 491-507 |

| | |
|---|---|
| PERIPHERAL | Likelihood = 0.69 (at 394) |

| | ALOM score: −8.60 (number of TMSs: 9) |
|---|---|
| MTOP: | Prediction of membrane topology (Hartmann et al.) |
| | Center position for calculation: 63 |
| | Charge difference: 0.0 C(0.5) − N(0.5) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 10.11 |
| Hyd Moment(95): | 10.29 | G content: | 1 |
| D/E content: | 1 | S/T content: | 5 |
| Score: −1.10 | | | |

Gavel: prediction of cleavage sites for mitochondrial preseq

R-2 motif at 421 PRL|AQ

NUCDISC: discrimination of nuclear localization signals

pat4: none
pat7: none
bipartite: none
content of basic residues: 4.6%
NLS Score: −0.47

KDEL: ER retention motif in the C-terminus: none
ER Membrane Retention Signals:

XXRR-like motif in the N-terminus: SRSP

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***

LDVLLHSLLTQPIFLAGLSGFL at 485

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

77.8%: endoplasmic reticulum
11.1%: nuclear
11.1%: mitochondrial

>> prediction for CG92078-02 is end (k = 9)

A search of the NOV76a protein against the Geneseq database, a proprietary database that contains sequences published in patents and patent publication, yielded several homologous proteins shown in Table 76D.

TABLE 76D
Geneseq Results for NOV76a

| | | NOV76a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE22904 | Human transporter and ion channel | 1 . . . 610 | 609/610 (99%) | 0.0 |
| | (TRICH) 3 - Homo sapiens, 610 | 1 . . . 610 | 610/610 (99%) | |
| | aa. [WO200222684-A2, 21-MAR- | | | |
| | 2002] | | | |
| ABB77088 | Human transporter protein related | 1 . . . 610 | 609/610 (99%) | 0.0 |
| | to permease subfamily - Homo | 1 . . . 610 | 610/610 (99%) | |
| | sapiens, 610 aa. [US2002028915- | | | |
| | A1, 07-MAR-2002] | | | |
| AAE29905 | Human transporter and ion channel | 1 . . . 610 | 607/618 (98%) | 0.0 |
| | (TRICH) protein #5 - Homo | 1 . . . 618 | 609/618 (98%) | |
| | sapiens, 618 aa. [WO200277237- | | | |
| | A2, 03-OCT-2002] | | | |
| AAW73924 | Nucleobase permease Yspl1 - Mus | 1 . . . 609 | 476/613 (77%) | 0.0 |
| | sp, 611 aa. [US5858707-A, 12- | 1 . . . 611 | 519/613 (84%) | |
| | JAN-1999] | | | |
| ABG70340 | Human MDDT protein Incyte ID | 1 . . . 610 | 390/611 (63%) | 0.0 |
| | No: LI:230711.5.orf2:2001JAN12 - | 12 . . . 519 | 413/611 (66%) | |
| | Homo sapiens, 519 aa. | | | |
| | [WO200255738-A2, 18-JUL- | | | |
| | 2002] | | | |

In a BLAST search of public sequence databases, the NOV76a protein was found to have homology to the proteins shown in the BLASTP data in Table 76E.

TABLE 76E
Public BLASTP Results for NOV76a

| | | NOV76a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q60850 | Yolk SAC permease-like YSPL-1 | 1 . . . 609 | 476/613 (77%) | 0.0 |
| | form 1 (Yolk SAC permease-like | 1 . . . 611 | 519/613 (84%) | |
| | YSPL-1 form 4) (Yolk SAC | | | |
| | permease-like YSPL-1 form 3) | | | |
| | (Yolk SAC permease-like YSPL-1 | | | |
| | form 2) - Mus musculus (Mouse), | | | |
| | 611 aa. | | | |
| CAC51146 | Sequence 22 from Patent | 1 . . . 227 | 225/227 (99%) | e−130 |
| | WO0149728 - Homo sapiens | 1 . . . 227 | 226/227 (99%) | |
| | (Human), 243 aa. | | | |
| AAH30243 | Similar to sodium-coupled | 1 . . . 225 | 225/225 (100%) | e−130 |
| | ascorbic acid transporter 2 - Homo | 1 . . . 225 | 225/225 (100%) | |
| | sapiens (Human), 492 aa. | | | |
| Q96NA6 | Hypothetical protein FLJ31168 - | 1 . . . 227 | 194/227 (85%) | e−107 |
| | Homo sapiens (Human), 212 aa. | 1 . . . 196 | 195/227 (85%) | |
| Q9WTW8 | Solute carrier family 23, member | 44 . . . 520 | 163/496 (32%) | 3e−76 |
| | 2 (Sodium-dependent vitamin C | 42 . . . 523 | 271/496 (53%) | |
| | transporter 2) (Na(+)/L-ascorbic | | | |
| | acid transporter 2) - Rattus | | | |
| | norvegicus (Rat), 592 aa. | | | |

PFam analysis indicates that the NOV76a protein contains the domains shown in the Table 76F.

TABLE 76F
Domain Analysis of NOV76a

| | | Identities/ | |
|---|---|---|---|
| | | Similarities | |
| Pfam Domain | NOV76a Match Region | for the Matched Region | Expect Value |
| xan_ur_permease | 46 . . . 473 | 116/461 (25%) | 5e−87 |
| | | 336/461 (73%) | |

### Example 77

The NOV77 clone was analyzed, and the nucleotide and encoded polypeptide sequences are shown in Table 77A.

TABLE 77A
NOV77 Sequence Analysis

| | | |
|---|---|---|
| NOV77a, CG93669-04 | SEQ ID NO: 1097 | 1542 bp |
| DNA Sequence | ORF Start: ATG at 1 | ORF Stop: at 1468 |

ATGGATGACTACATGGTCCTGAGAATGATTGGGGAGGGCTCCTTCGGCAGAGCTCTTTTGGTTCAGCA
TGAAAGCAGTAATCAGATGTTTGCCATGAAAGAAATAAGGCTTCCCAAGTCTAATACACAGAATTCTA
GGAAGGAGGCTGTTCTTTTAGCCAAAATGAAACACCCTAATATTGTTGCCTTCAAAGAATCATTTGAA
GCTGAAGGACACTTGTATATTGTGATGGAATACTGTGATGGAGGGGATCTAATGCAAAAGATTAAACA
GCAGAAAGGAAAGTTATTTCCTGAAGACATACTTAATTGGTTTACCCAAATGTGCCTTGGAGTAAATC
ACATTCACAAGAAACGTGTGCTACACAGAGATATCAAGTCCAAGAATATCTTCCTCACTCAGAATGGA
AAAGTGAAATTGGGAGACTTTGGATCTGCCCGTCTTCTCTCCAGTCCGATGGCATTTGCTTGTACCTA
TGTGGGAACTCCTTATTATGTGCCTCCAGAAATTTGGGAAAACCTGCCTTATAACAATAAAAGTGACA
TCTGGTCCTTGGGTTGCATCCTGTATGAACTCTGTACCCTTAAGCATCCATTTCAGGCAAATAGTTGG
AAAAATCTTATCCTCAAAGTATGTCAAGGGTGCATCAGTCCACTGCCGTCTCATTACTCCTATGAACT
TCAGTTCCTAGTCAAGCAGATGTTTAAAAGGAATCCCTCACATCGCCCCTCGGCTACAACGCTTCTCT
CTCGAGGCATCGTAGCTCGGCTTGTCCAGAAGTGCTTACCCCCCCAGATCATCATGGAATATGGTGAG
GAAGTATTAGAAGAAATAAAAAATTCGAAGCATAACACACCAAGAAAAAAATCTCTTTTAAAGCAAGA
GGAAGAACAAGATAGAAAGGGTAGCCATACTGATTTGGAAAGCATTAATGAAAATTTAGTTGAAAGTG
CATTGAGAAGAGTAAACAGAAAATCAGGTAATAAGTCAGTCCATCTGAGGAAAGCCAGTTCACCAAAT
CTTCATAGACGACAGTGGGAGAAAAATGTACCCAATACAGCTCTTACAGCTTTGGAAAATGCATCCAT
ACTCACCTCCAGTTTAACAGCAGAGGACGATAGAGGTGGTGGTTCTGTAATAAAGTACAGCAAAAATA
CTACTCGTAAGCAGTGGCTCAAAGAGACCCCTGACACTTTGTTGAACATCCTTAAGAATGCTGATCTC
AGCTTGGCTTTTCAAACATACACAATATATAGACCAGGTTCAGAAGGGTTCTTGAAAGGCCCCCTGTC
TGAAGAAACAGAAGCATCGGACAGTGTTGATGGAGGTCACGATTCTGTCATTTTGGATCCAGAGCGAC
TTGAGCCTGGGCTAGATGAGGAGGACACGGACTTTGAGGAGGAAGATGACAACCCCGACTGGGTGTCA
GAGCTGAAGAAGCGAGCTGGATGGCAAGGCCTGTGCGACAGATAATGCCTGAGGAAATGTTCCTGAGT
CACGCTGAGGAGAGGCTTCACTCAGGAGTTCATGCTGAGATGATCA

| | | | |
|---|---|---|---|
| NOV77a, CG93669-04 | SEQ ID NO: 1098 | 489 aa | MW at 55700.9kD |
| Protein Sequence | | | |

MDDYMVLRMIGEGSFGRALLVQHESSNQMFANKEIRLPKSNTQNSRKEAVLLAKMKHPNIVAFKESFE
AEGHLYIVMEYCDGGDLMQKIKQQKGKLFPEDILNWFTQMCLGVNHIHKKRVLHRDIKSKNIFLTQNG
KVKLGDFGSARLLSSPMAFACTYVGTPYYVPPEIWENLPYNNKSDIWSLGCILYELCTLKHPFQANSW
KNLILKVCQGCISPLPSHYSYELQFLVKQMFKRNPSHRPSATTLLSRGIVARLVQKCLPPQIIMEYGE
EVLEEIKNSKHNTPRKKSLLKQEEEQDRKGSHTDLESINENLVESALRRVNRKSGNKSVHLRKASSPN
LHRRQWEKNVPNTALTALENASILTSSLTAEDDRGGGSVIKYSKNTTRKQWLKETPDTLLNILKNADL
SLAFQTYTIYRPGSEGFLKGPLSEETEASDSVDGGHDSVILDPERLEPGLDEEDTDFEEEDDNPDWVS
ELKKRAGWQGLCD

| | | |
|---|---|---|
| NOV77b, CG93669-01 | SEQ ID NO: 1099 | 2257 bp |
| DNA Sequence | ORF Start: ATG at 246 | ORF Stop: TAA at 1764 |

CCGCAAGTCCCTCGCCGCCTTGGGGTCTGGGCGCGCGGTCCGTGGGGGTCAGCAGGGCGAGCGGCTTT
TCCAGGAGAAAGGGCCCTCACGGGTGAGCGGGGCGACTGGGCTCCCCCGCGGTGCAGTTGCCCCGCGC
GACCGGCCCCGGCTTCAACGGATTCTTCTCGCTCGCTGCCCGGAAAGAACCATTTGGGAGAGCCCATG
GTGACTGCGTGAGTGGAGCCCAGCTGTGTGGATGCCCCAGCATGGATGACTACATGGTCCTGAGAATG
ATTGGGGAGGGCTCCTTCGGCAGAGCTCTTTTGGTTCAGCATGAAAGCAGTAATCAGATGTTTGCCAT
GAAAGAAATAAGGCTTCCCAAGGTCACTACTAATACACAGAATTCTAGGAAGGAGGCTGTTCTTTTAG
CCAAAATGAAACACCCTAATATTGTTGCCTTCAAAGAATCATTTGAAGCTGAAGGACACTTGTATATT
GTGATGGAATACTGTGATGGAGGGGATCTAATGCAAAAGATTAAACAGCAGAAAGGAAAGTTATTTCC
TGAAGACCAGATACTTAATTGGTTTACCCAAATGTGCCTTGGAGTAAATCACATTCACAAGAAACGTG
TGCTACACAGAGATATCAAGTCCCAGAATATCTTCCTCACTCAGAATGGAAAAGTGAAATTGGGAGAC
TTTGGATCTGCCCGTCTTCTCTCCAATCCGATGGCATTTGCTTGTACCTATGTGGGAACTCCTTATTA
TGTGCCTCCAGAAATTTGGGAAAACCTGCCTTATAACAATAAAAGTGACATCTGGTCCTTGGGTTGCA
TCCTGTATGAACTCTGTACCCTTAAGCATCCATTTCAGGCAAATAGTTGGAAAAATCTTATCCTCAAA
GTATGTCAAGGGTGCATCAGTCCACTGCCGTCTCATTACTCCTATGAACTTCAGTTCCTAGTCAAGCA
GATGTTTAAAAGGAATCCCTCACATCGCCCCTCGGCTACAACGCTTCTCTCTCGAGGCATCGTAGCTC
GGCTTGTCCAGAAGTGCTTACCCCCCGAGATCATCATGGAATATGGTGAGGAAGTATTAGAAGAAATA
AAAAATTCGAAGCATAACACACCAAGAAAAAAAACAAACCCCAGCAGAATCAGGATAGCTTTGGGAAA
TGAAGCAAGCACAGTGCAAGAGGAAGAACAAGATAGAAAGGGTAGCCATACTGATTTGGAAAGCATTA
ATGAAAATTTAGTTGAAAGTGCATTGAGAAGAGTAAACAGAGAAGAAAAAGGTAATAAGTCAGTCCAT
CTGAGGAAAGCCAGTTCACCAAATCTTCATAGACGACAGTGGGAGAAAAATGTACCCAATACAGCTCT
TACAGCTTTGGAAAATGCATCCATACTCACCTCCAGTTTAACAGCAGAGGACGATAGAGGTGGTTCTG
TAATAAAGTACAGCAAAAATACTACTCGTAAGCAGTGGCTCAAAGAGACCCCTGACACTTTGTTGAAC
ATCCTTAAGAATGCTGATCTCAGCTTGGCTTTTCAAACATACACAATATATAGACCAGGTTCAGAAGG
GTTCTTGAAAGGCCCCCTGTCTGAAGAAACAGAAGCATCGGACAGTGTTGATGGAGGTCACGATTCTG
TCATTTTGGATCCAGAGCGACTTGAGCCTGGGCTAGATGAGGAGGACACGGACTTTGAGGAGGAAGAT
GACAACCCCGACTGGGTGTCAGAGCTGAAGAAGCGAGCTGGATGGCAAGGCCTGTGCGACAGATAATG
CCTGAGGAAATGTTCCTGAGTCACGCTGAGGAGAGCCTTCACTCAGGAGTTCATGCTGAGATGATCAT
GAGTTCATGCGACGTATATTTTCCTTTGGAAACAGAATGAAGCAGAGGAAACTCTTAATACTTAAAAT
CGTTCTTGATTAGTATCGTGAGTTTGAAAAGTCTAGAACTCCTGTAAGTTTTTGAACTCAAGGGAGAA
GGTATAGTGGAATGAGTGTGAGCATCGGGCTTTGCAGTCCCATAGAACAGAAATGGGATGCTAGCGTG
CCACTACCTACTTGTGTGATTGTGGGAAATTACTTAACCTCTTCAAGCCCCAATTTCCTCAACCATAA
AATGAAGATAATAATGCCTACCTCAGAGGGATGCTGACCACAGACCTTTATAGCAGCCCGTATGATAT
TATTCACATTATGATATGTGTTTATTATTATGTGACTCTTTTTACATTTCCTAAAGGTTTGAGAATTA
AATATATTTAATT

| | | | |
|---|---|---|---|
| NOV77b, CG93669-01 | SEQ ID NO: 1100 | 506 aa | MW at 57681.0kD |
| Protein Sequence | | | |

MDDYMVLRMIGEGSFGRALLVQHESSNQMFA4KEIRLPKVTTNTQMSRKEAVLLAKMKHPNIVAFKES
FEAEGHLYIVMEYCDGGDLMQKIKQQKGKLFPEDQILNWFTQMCLGVNHIHKKRVLHRDIKSQNIFLT
QNGKVKLGDFGSAPLLSNPMAFACTYVGTPYYVPPEIWENLPYNNKSDIWSLGCILYELCTLKHPFQA
NSWKNLILKVCQGCISPLPSHYSYELQFLVKQMFKRNPSHRPSATTLLSRGIVARLVQKCLPPEIIME
YGEEVLEEIKNSKHNTPRKKTNPSRIRIALGNEASTVQEEEQDRKGSHTDLESINENLVESALRRVNR
EEKGNKSVHLRKASSPNLHRRQWEKNVPNTALTALENASILTSSLTAEDDRGGSVIKYSKNTTRKQWL
KETPDTLLNILKNADLSLAFQTYTIYRPGSEGFLKGPLSEETEASDSVDGGHDSVILDPERLEPGLDE
EDTDFEEEDDNPDWVSELKKRAGWQGLCDR

| | | |
|---|---|---|
| NOV77c,CG93669-02 | SEQ ID NO: 1101 | 1781 bp |
| DNA Sequence | ORF Start: ATG at 246 | ORF Stop: TAA at 1713 |

CCGCAAGTCCCTCGCCGCCTTGGGGTCTGGGCGCGCGGTCCGTGGGGGTCAGCAGGGCGAGCGGCTTT
TCCAGGAGAAAGGGCCCTCACGGGTGAGCGGGGCGACTGGGCTCCCCCGCGGTGCAGTTGCCCCGCGC
GACCGGCCCCGGCTTCAACGGATTCTTCTCGCTCGCTGCCCGGAAAGAACCATTTGGGAGAGCCCATG
GTGACTGCGTGAGTGGAGCCCAGCTGTGTGGATGCCCCAGCATGGATGACTACATGGTCCTGAGAATG
ATTGGGGAGGGCTCCTTCGGCAGAGCTCTTTTGGTTCAGCATGAAAGCAGTAATCAGATGTTTGCCAT
GAAAGAAATAAGGCTTCCCAAGGTCACTACTAATACACAGAATTCTAGGAAGGAGGCTGTTCTTTTAG
CCAAAATGAAACACCCTAATATTGTTGCCTTCAAAGAATCATTTGAAGCTGAAGGACACTTGTATATT
GTGATGGAATACTGTGATGGAGGGGATCTAATGCAAAAGATTAAACAGCAGAAAGGAAAGTTATTTCC
TGAAGACCAGATACTTAATTGGTTTACCCAAATGTGCCTTGGAGTAAATCACATTCACAAGAAACGTG
TGCTACACAGAGATATCAAGTCCCAGAATATCTTCCTCACTCAGAATGGAAAAGTGAAATTGGGAGAC
TTTGGATCTGCCCGTCTTCTCTCCAATCCGATGGCATTTGCTTGTACCTATGTGGGAACTCCTTATTA
TGTGCCTCCAGAAATTTGGGAAAACCTGCCTTATAACAATAAAAGTGACATCTGGTCCTTGGGTTGCA
TCCTGTATGAACTCTGTACCCTTAAGCATCCATTTCAGGCAAATAGTTGGAAAAATCTTATCCTCAAA
GTATGTCAAGGGTGCATCAGTCCACTGCCGTCTCATTACTCCTATGAACTTCAGTTCCTAGTCAAGCA
GATGTTTAAAAGGAATCCCTCACATCGCCCCTCGGCTACAACGCTTCTCTCTCGAGGCATCGTAGCTC
GGCTTGTCCAGAAGTGCTTACCCCCCGAGATCATCATGGAATATGGTGAGGAAGTATTAGAAGAAATA
AAAAATTCGAAGCATAACACACCAAGAAAAAAACAAGAGGAAGAACAAGATAGAAAGGGTAGCCATAC
TGATTTGGAAAGCATTAATGAAAATTTAGTTGAAAGTGCATTGAGAAGAGTAAACAGAGAAGAAAAAG
GTAATAAGTCAGTCCATCTGAGGAAAGCCAGTTCACCAAATCTTCATAGACGACAGTGGGAGAAAAAT
GTACCCAATACAGCTCTTACAGCTTTGGAAAATGCATCCATACTCACCTCCAGTTTAACAGCAGAGGA
CGATAGAGGTGGTTCTGTAATAAAGTACAGCAAAAATACTACTCGTAAGCAGTGGCTCAAAGAGACCC
CTGACACTTTGTTGAACATCCTTAAGAATGCTGATCTCAGCTTGGCTTTTCAAACATACACAATATAT
AGACCAGGTTCAGAAGGGTTCTTGAAAGGCCCCCTGTCTGAAGAAACAGAAGCATCGGACAGTGTTGA
TGGAGGTCACGATTCTGTCATTTTGGATCCAGAGCGACTTGAGCCTGGGCTAGATGAGGAGGACACGG
ACTTTGAGGAGGAAGATGACAACCCCGACTGGGTGTCAGAGCTGAAGAAGCGAGCTGGATGGCAAGGC
CTGTGCGACAGATAATGCCTGAGGAAATGTTCCTGAGTCACGCTGAGGAGAGGCTTCACTCTAGGAGT
TCATGCTGAGATG

| | | | |
|---|---|---|---|
| NOV77c, CG93669-02 | SEQ ID NO: 1102 | 489 aa | MW at 55900.0kD |
| Protein Sequence | | | |

MDDYMVLRMIGEGSFGRALLVQHESSNQMFANKEIRLPKVTTNTQNSRKEAVLLAKMKHPNIVAFKES
FEAEGHLYIVMEYCDGGDLMQKIKQQKGKLFPEDQILNWFTQMCLGVNHIHKKRVLHRDIKSQNIFLT
QNGKVKLGDFGSARLLSNPMAFACTYVGTPYYVPPEIWENLPYNNKSDIWSLGCILYELCTLKHPFQA
NSWKNLILKVCQGCISPLPSHYSYELQFLVKQNFKRNPSHRPSATTLLSRGIVARLVQKCLPPEIIME
YGEEVLEEIKNSKHNTPRKKQEEEQDRKGSHTDLESINENLVESALRRVNREEKGNKSVHLRKASSPN
LHRRQWEKNVPNTALTALENASILTSSLTAEDDRGGSVIKYSKNTTRKQWLKETPDTLLNILKNADLS
LAFQTYTIYRPGSEGFLKGPLSEETEASDSVDGGHDSVILDPERLEPGLDEEDTDFEEEDDNPDWVSE
LKKRAGWQGLCDR

| | | |
|---|---|---|
| NOV77d, GG93669-03 | SEQ ID NO: 1103 | 1588 bp |
| DNA Sequence | ORF Start: ATG at 246 | ORF Stop: TAA at 1521 |

CCGCAAGTCCCTCGCCGCCTTGGGGTCTGGGCGCGCGGTCCGTGGGGGTCAGCAGGGCGAGCGGCTTT
TCCAGGAGAAAGGGCCCTCACGGGTGAGCGGGGCGACTGGGCTCCCCCGCGGTGCAGTTGCCCCGCGC
GACCGGCCCCGGCTTCAACGGATTCTTCTCGCTCGCTGCCCGGAAAGAACCATTTGGGAGAGCCCATG
GTGACTGCGTGAGTGGAGCCCAGCTGTGTGGATGCCCCAGCATGGATGACTACATGGTCCTGAGAATG
ATTGGGGAGGGCTCCTTCGGCAGAGCTCTTTTGGTTCAGCATGAAAGCAGTAATCAGATGTTTGCCAT
GAAAGAAATAAGGCTTCCCAAGGTCACTACTAATACACAGAATTCTAGGAAGGAGGCTGTTCTTTTAG
CCAAAATGAAACACCCTAATATTGTTGCCTTCAAAGAATCATTTGAAGCTGAAGGACACTTGTATATT
GTGATGGAATACTGTGATGGAGGGGATCTAATGCAAAAGATTAAACAGCAGAAAGGAAAGTTATTTCC
TGAAGACCAGATACTTAATTGGTTTACCCAAATGTGCCTTGGAGTAAATCACATTCACAAGAAACGTG
TGCTACACAGAGATATCAAGTCCCAGAATATCTTCCTCACTCAGAATGGAAAAGTGAAATTGGGAGAC
TTTGGATCTGCCCGTCTCCTCTCCAATCCGATGGCATTTGCTTGTACCTATGTGGGAACTCCTTATTA
TGTGCCTCCAGAAATTTGGGAAAACCTGCCTTATAACAATAAAAGTGACATCTGGTCCTTGGGTTGCA
TCCTGTATGAACTCTGTACCCTTAAGCATCCATTTCAGGCAAATAGTTGGAAAAATCTTATCCTCAAA
GTATGTCAAGGGTGCATCAGTCCACTGCCGTCTCATTACTCCTATGAACTTCAGTTCCTAGTCAAGCA
GATGTTTAAAAGGAATCCCTCACATCGCCCCTCGGCTACAGCGCTTCTCTCTCGAGGCATCGTAGCTC
GGCTTGTCCAGAAGTGCTTACCCCCCGAGATCATCATGGAATATGGTGAGGAAGTATTAGAAGAAATA
AAAAATTCGAAGCATAACACACCAAGAAAAAAACAAGAGGAAGAACAAGATAGAAAGGGTAGCCATAC
TGATTTGGAAAGCATTAATGAAAATTTAGTTGAAAGTGCATTGAGAAGAGTAAACAGAGAAGAAAAAG
GTAATAAGTCAGTCCATCTGAGGAAAGCCAGTTCACCAAATCTTCATAGACGACAGTGGGAGAAAAAT
GTACCCAATACAGCTCTTACAGCTTTGGAAAATGCATCCATACTCACCTCCAGTTTAACAGCAGAGGA
CGATAGAGGTTCAGAAGGGTTCTTGAAAGGCCCCCTGTCTGAAGAAACAGAAGCATCGGACAGTGTTG
AGGAGGACACGGACTTTGAGGAGGAAGATGACAACCCCGACTGGGTGTCAGAGCTGAAGAAGCGAGCT
GGATGGCAAGGCCTGTGCGACAGATAATGCCTGAGGAAATGTACCTGAGTCACGCTGAGGAGAGGCTT
CACTCAGGAGTTCATGCTGAGATG

| | | | |
|---|---|---|---|
| NOV77d, CG93669-03 | SEQ ID NO: 1104 | 425 aa | MW at 48684.0kD |
| Protein Sequence | | | |

MDDYMVLRMIGEGSFGRALLVQHESSNQMFAMKEIRLPKVTTNTQNSRKEAVLLAKMKHPNIVAFKES
FEAEGHLYIVMEYCDGGDLMQKIKQQKGKLFPEDQILNWFTQMCLGVNHIHKKRVLHRDIKSQNIFLT
QNGKVKLGDFGSARLLSNPMAFACTYVGTPYYVPPEIWENLPYNNKSDIWSLGCILYELCTLKHPFQA
NSWKNLILKVCQGCISPLPSHYSYELQFLVKQMFKRNPSHRPSATALLSRGIVARLVQKCLPPEIIME
YGEEVLEEIKNSKHNTPRKKQEEEQDRKGSHTDLESINENLVESALRRVNREEKGNKSVHLRKASSPN
LHRRQWEKNVPNTALTALENASILTSSLTAEDDRGSEGFLKGPLSEETEASDSVEEDTDFEEEDDNPD
WVSELKKRAGWQGLCDR

| | | |
|---|---|---|
| NOV77e, SNP13376464 | SEQ ID NO: 1105 | 1542 bp |
| of CG93669-04, | ORF Start: ATG at 1 | ORF Stop: at 1468 |
| DNA Sequence | SNP Pos: 94 | SNP Change: A to G |

ATGGATGACTACATGGTCCTGAGAATGATTGGGGAGGGCTCCTTCGGCAGAGCTCTTTTGGTTCAGCA
TGAAAGCAGTAATCAGATGTTTGCCGTGAAAGAAATAAGGCTTCCCAAGTCTAATACACAGAATTCTA
GGAAGGAGGCTGTTCTTTTAGCCAAAATGAAACACCCTAATATTGTTGCCTTCAAAGAATCATTTGAA
GCTGAAGGACACTTGTATATTGTGATGGAATACTGTGATGGAGGGGATCTAATGCAAAAGATTAAACA
GCAGAAAGGAAAGTTATTTCCTGAAGACATACTTAATTGGTTTACCCAAATGTGCCTTGGAGTAAATC
ACATTCACAAGAAACGTGTGCTACACAGAGATATCAAGTCCAAGAATATCTTCCTCACTCAGAATGGA
AAAGTGAAATTGGGAGACTTTGGATCTGCCCGTCTTCTCTCCAGTCCGATGGCATTTGCTTGTACCTA
TGTGGGAACTCCTTATTATGTGCCTCCAGAAATTTGGGAAAACCTGCCTTATAACAATAAAAGTGACA
TCTGGTCCTTGGGTTGCATCCTGTATGAACTCTGTACCCTTAAGCATCCATTTCAGGCAAATAGTTGG
AAAAATCTTATCCTCAAAGTATGTCAAGGGTGCATCAGTCCACTGCCGTCTCATTACTCCTATGAACT
TCAGTTCCTAGTCAAGCAGATGTTTAAAAGGAATCCCTCACATCGCCCCTCGGCTACAACGCTTCTCT
CTCGAGGCATCGTAGCTCGGCTTGTCCAGAAGTGCTTACCCCCCCAGATCATCATGGAATATGGTGAG
GAAGTATTAGAAGAAATAAAAAATTCGAAGCATAACACACCAAGAAAAAAATCTCTTTTAAAGCAAGA
GGAAGAACAAGATAGAAAGGGTAGCCATACTGATTTGGAAAGCATTAATGAAAATTTAGTTGAAAGTG
CATTGAGAAGAGTAAACAGAAAATCAGGTAATAAGTCAGTCCATCTGAGGAAAGCCAGTTCACCAAAT
CTTCATAGACGACAGTGGGAGAAAAATGTACCCAATACAGCTCTTACAGCTTTGGAAAATGCATCCAT
ACTCACCTCCAGTTTAACAGCAGAGGACGATAGAGGTGGTGGTTCTGTAATAAAGTACAGCAAAAATA
CTACTCGTAAGCAGTGGCTCAAAGAGACCCCTGACACTTTGTTGAACATCCTTAAGAATGCTGATCTC
AGCTTGGCTTTTCAAACATACACAATATATAGACCAGGTTCAGAAGGGTTCTTGAAAGGCCCCCTGTC
TGAAGAAACAGAAGCATCGGACAGTGTTGATGGAGGTCACGATTCTGTCATTTTGGATCCAGAGCGAC
TTGAGCCTGGGCTAGATGAGGAGGACACGGACTTTGAGGAGGAAGATGACAACCCCGACTGGGTGTCA
GAGCTGAAGAAGCGAGCTGGATGGCAAGGCCTGTGCGACAGATAATGCCTGAGGAAATGTTCCTGAGT
CACGCTGAGGAGAGGCTTCACTCAGGAGTTCATGCTGAGATGATCA

| | | | |
|---|---|---|---|
| NOV77e, SNP13376464 | SEQ ID NO: 1106 | 489 aa | MW at 55668.8kD |
| of CG93669-04, | SNP Change: 32 | | SNP Change: Met to Val |
| Protein Sequence | | | |

MDDYMVLRMIGEGSFGRALLVQHESSNQMFAVKEIRLPKSNTQNSRKEAVLLAKMKHPNIVAFKESFE
AEGHLYIVMEYCDGGDLMOKIKQQKGKLFPEDILNWFTQMCLGVNHIHKKRVLHRDIKSKNIFLTQNG
KVKLGDFGSARLLSSPMAFACTYVGTPYYVPPEIWENLPYNNKSDIWSLGCILYELCTLKHPFQANSW
KNLILKVCQGCISPLPSHYSYELQFLVKQMFKRNPSHRPSATTLLSRGIVARLVQKCLPPQIIMEYGE
EVLEEIKNSKHNTPRKKSLLKQEEEQDRKGSRTDLESINENLVESALRRVNRKSGNKSVHLRKASSPN
LHRRQWEKNVPNTALTALENASILTSSLTAEDDRGGGSVIKYSKNTTRKQWLKETPDTLLNILKNADL
SLAFQTYTIYRPGSEGFLKGPLSEETEASDSVDGGHDSVILDPERLEPGLDEEDTDFEEEDDNPDWVS
ELKKRAGWQGLCD

| | | |
|---|---|---|
| NOV77f, SNP13376462 | SEQ ID NO: 1107 | 1542 bp |
| of CG93669-04, | ORF Start: ATG at 1 | ORF Stop: at 1468 |
| DNA Sequence | SNP Pos: 284 | SNP Change: A to G |

ATGGATGACTACATGGTCCTGAGAATGATTGGGGAGGGCTCCTTCGGCAGAGCTCTTTTGGTTCAGCA
TGAAAGCAGTAATCAGATGTTTGCCATGAAAGAAATAAGGCTTCCCAAGTCTAATACACAGAATTCTA
GGAAGGAGGCTGTTCTTTTAGCCAAAATGAAACACCCTAATATTGTTGCCTTCAAAGAATCATTTGAA
GCTGAAGGACACTTGTATATTGTGATGGAATACTGTGATGGAGGGGATCTAATGCAAAAGATTAAACA
GCAGAAAGGAAGGTTATTTCCTGAAGACATACTTAATTGGTTTACCCAAATGTGCCTTGGAGTAAATC
ACATTCACAAGAAACGTGTGCTACACAGAGATATCAAGTCCAAGAATATCTTCCTCACTCAGAATGGA
AAAGTGAAATTGGGAGACTTTGGATCTGCCCGTCTTCTCTCCAGTCCGATGGCATTTGCTTGTACCTA
TGTGGGAACTCCTTATTATGTGCCTCCAGAAATTTGGGAAAACCTGCCTTATAACAATAAAAGTGACA
TCTGGTCCTTGGGTTGCATCCTGTATGAACTCTGTACCCTTAAGCATCCATTTCAGGCAAATAGTTGG
AAAAATCTTATCCTCAAAGTATGTCAAGGGTGCATCAGTCCACTGCCGTCTCATTACTCCTATGAACT
TCAGTTCCTAGTCAAGCAGATGTTTAAAAGGAATCCCTCACATCGCCCCTCGGCTACAACGCTTCTCT
CTCGAGGCATCGTAGCTCGGCTTGTCCAGAAGTGCTTACCCCCCCAGATCATCATGGAATATGGTGAG
GAAGTATTAGAAGAAATAAAAAATTCGAAGCATAACACACCAAGAAAAAAATCTCTTTTAAAGCAAGA
GGAAGAACAAGATAGAAAGGGTAGCCATACTGATTTGGAAAGCATTAATGAAAATTTAGTTGAAAGTG
CATTGAGAAGAGTAAACAGAAAATCAGGTAATAAGTCAGTCCATCTGAGGAAAGCCAGTTCACCAAAT
CTTCATAGACGACAGTGGGAGAAAAATGTACCCAATACAGCTCTTACAGCTTTGGAAAATGCATCCAT
ACTCACCTCCAGTTTAACAGCAGAGGACGATAGAGGTGGTGGTTCTGTAATAAAGTACAGCAAAAATA
CTACTCGTAAGCAGTGGCTCAAAGAGACCCCTGACACTTTGTTGAACATCCTTAAGAATGCTGATCTC
AGCTTGGCTTTTCAAACATACACAATATATAGACCAGGTTCAGAAGGGTTCTTGAAAGGCCCCCTGTC
TGAAGAAACAGAAGCATCGGACAGTGTTGATGGAGGTCACGATTCTGTCATTTTGGATCCAGAGCGAC
TTGAGCCTGGGCTAGATGAGGAGGACACGGACTTTGAGGAGGAAGATGACAACCCCGACTGGGTGTCA
GAGCTGAAGAAGCGAGCTGGATGGCAAGGCCTGTGCGACAGATAATGCCTGAGGAAATGTTCCTGAGT
CACGCTGAGGAGAGGCTTCACTCAGGAGTTCATGCTGAGATGATCA

| | | | |
|---|---|---|---|
| NOV77f, SNP13376462 | SEQ ID NO: 1108 | 489 aa | MW at 55728.9kD |
| of CG93669-04, | SNP Pos: 95 | | SNP Change: Lys to Arg |
| Protein Sequence | | | |

MDDYMVLRMIGEGSFGRALLVQHESSNQMFANKEIRLPKSNTQNSRKEAVLLAKMKHPNIVAFKESFE
AEGHLYIVMEYCDGGDLMQKIKQQKGRLFPEDILNWFTQMCLGVNHIHKKRVLHRDIKSKNIFLTQNG
KVKLGDFGSARLLSSPMAFACTYVGTPYYVPPEIWENLPYNNKSDIWSLGCILYELCTLKHPFQANSW
KNLILKVCQGCISPLPSHYSYELQFLVKQMFKRNPSHRPSATTLLSRGIVARLVQKCLPPQIIMEYGE
EVLEEIKNSKHNTPRKKSLLKQEEEQDRKGSHTDLESINENLVESALRRVNRKSGNKSVHLRKASSPN
LHRRQWEKNVPNTALTALENASILTSSLTAEDDRGGGSVIKYSKNTTRKQWLKETPDTLLNILKNADL
SLAFQTYTIYRPGSEGFLKGPLSEETEASDSVDGGHDSVILDPERLEPGLDEEDTDFEEEDDNPDWVS
ELKKRAGWQGLCD

| | | |
|---|---|---|
| NOV77g, SNP13382521 | SEQ ID NO: 1109 | 1542 bp |
| of CG93669-04, | ORF Start: ATG at 1 | ORF Stop: at 1468 |
| DNA Sequence | SNP Pos: 1511 | SNP Change: G to C |

ATGGATGACTACATGGTCCTGAGAATGATTGGGGAGGGCTCCTTCGGCAGAGCTCTTTTGGTTCAGCA
TGAAAGCAGTAATCAGATGTTTGCCATGAAAGAAATAAGGCTTCCCAAGTCTAATACACAGAATTCTA
GGAAGGAGGCTGTTCTTTTAGCCAAAATGAAACACCCTAATATTGTTGCCTTCAAAGAATCATTTGAA
GCTGAAGGACACTTGTATATTGTGATGGAATACTGTGATGGAGGGGATCTAATGCAAAAGATTAAACA
GCAGAAAGGAAAGTTATTTCCTGAAGACATACTTAATTGGTTTACCCAAATGTGCCTTGGAGTAAATC
ACATTCACAAGAAACGTGTGCTACACAGAGATATCAAGTCCAAGAATATCTTCCTCACTCAGAATGGA
AAAGTGAAATTGGGAGACTTTGGATCTGCCCGTCTTCTCTCCAGTCCGATGGCATTTGCTTGTACCTA
TGTGGGAACTCCTTATTATGTGCCTCCAGAAATTTGGGAAAACCTGCCTTATAACAATAAAAGTGACA
TCTGGTCCTTGGGTTGCATCCTGTATGAACTCTGTACCCTTAAGCATCCATTTCAGGCAAATAGTTGG
AAAAATCTTATCCTCAAAGTATGTCAAGGGTGCATCAGTCCACTGCCGTCTCATTACTCCTATGAACT
TCAGTTCCTAGTCAAGCAGATGTTTAAAAGGAATCCCTCACATCGCCCCTCGGCTACAACGCTTCTCT
CTCGAGGCATCGTAGCTCGGCTTGTCCAGAAGTGCTTACCCCCCCAGATCATCATGGAATATGGTGAG
GAAGTATTAGAAGAAATAAAAAATTCGAAGCATAACACACCAAGAAAAAAATCTCTTTTAAAGCAAGA
GGAAGAACAAGATAGAAAGGGTAGCCATACTGATTTGGAAAGCATTAATGAAAATTTAGTTGAAAGTG
CATTGAGAAGAGTAAACAGAAAATCAGGTAATAAGTCAGTCCATCTGAGGAAAGCCAGTTCACCAAAT
CTTCATAGACGACAGTGGGAGAAAAATGTACCCAATACAGCTCTTACAGCTTTGGAAAATGCATCCAT
ACTCACCTCCAGTTTAACAGCAGAGGACGATAGAGGTGGTGGTTCTGTAATAAAGTACAGCAAAAATA
CTACTCGTAAGCAGTGGCTCAAAGAGACCCCTGACACTTTGTTGAACATCCTTAAGAATGCTGATCTC
AGCTTGGCTTTTCAAACATACACAATATATAGACCAGGTTCAGAAGGGTTCTTGAAAGGCCCCCTGTC
TGAAGAAACAGAAGCATCGGACAGTGTTGATGGAGGTCACGATTCTGTCATTTTGGATCCAGAGCGAC
TTGAGCCTGGGCTAGATGAGGAGGACACGGACTTTGAGGAGGAAGATGACAACCCCGACTGGGTGTCA
GAGCTGAAGAAGCGAGCTGGATGGCAAGGCCTGTGCGACAGATAATGCCTGAGGAAATGTTCCTGAGT
CACGCTGAGGAGAGCCTTCACTCAGGAGTTCATGCTGAGATGATCA

| | | | |
|---|---|---|---|
| NOV77g, SNP13382521 | SEQ ID NO: 1110 | 489 aa | MW at 55700.9kD |
| of CG93669-04, | | | SNP Change: no change |
| Protein Sequence | | | |

MDDYMVLRNIGEGSFGRALLVQHESSNQMFAMKEIRLPKSNTQNSRKEAVLLAKMKHPNIVAFKESFE
AEGHLYIVMEYCDGGDLMQKIKQQKGKLFPEDILNWFTQMCLGVNHIHKKRVLHRDIKSKNIFLTQNG
KVKLGDFGSARLLSSPMAFACTYVGTPYYVPPEIWENLPYNNCSDIWSLGCILYELCTLKHPFQANSW
KNLILKVCQGCISPLPSHYSYELQFLVKQMFKRNPSHRPSATTLLSRGIVARLVQKCLPPQIIMEYGE
EVLEEIKNSKHNTPRKKSLLKQEEEQDRKGSHTDLESINENLVESALRRVNRKSGNKSVHLRKASSPN
LHRRQWEKNVPNTALTALENASILTSSLTAEDDRGGGSVIKYSKNTTRKQWLKETPDTLLNILKNADL
SLAFQTYTIYRPGSEGFLKGPLSEETEASDSVDGGHDSVILDPERLEPGLDEEDTDFEEEDDNPDWVS
ELKKRAGWQGLCD

A ClustalW comparison of the above protein sequences yields the following sequence alignment shown in Table 77B.

TABLE 77B
Comparison of the NOV77 protein sequences.

| | |
|---|---|
| NOV77a | MDDYMVLRMIGEGSFGRALLVQHESSNQMFAMKEIRLPKS--NTQNSRKEAVLLAKMKHP |
| NOV77b | MDDYMVLRMIGEGSFGRALLVQHESSNQMFAMKEIRLPKVTTNTQNSRKEAVLLAKMKMP |
| NOV77c | MDDYMVLRMIGEGSFGRALLVQHESSNQMFAMKEIRLPKVTTNTQNSRKEAVLLAKMKHP |
| NOV77d | MDDYMVLRMIGEGSFGRALLVOHESSNQMFAMKEIRLPKVTTNTQNSRKEAVLLAKMKHP |
| NOV77a | NIVAFKESFEAEGHLYIVMEYCDGGDLMQKIKQQKGKIFPED-ILNWFTQMCLGVNHIHK |
| NOV77b | NIVAFKESFEAEGHLYIVMEYCDGGDLMQKIKQQKGKIFPEDQILNWFTOMCLGVNHINK |
| NOV77c | NIVAFKESFEAEGHLYIVMEYCDGGDLMQKIKQQKGKLFPEDQILNWFTQMCLGVNHIHK |
| NOV77d | NIVAFKESFEAEGHLYIVMEYCDGGDLMQKIKQQKGKLFPEDQILNWFTQMCLGVNHIHK |
| NOV77a | KRVLHRDIKSKNIFLTQNGKVKLGDFGSARLLSSPMAFACTYVGTPYYVPPEIWENLPYN |
| NOV77b | KRVLHRDIKSQNIFLTQNGKVKLGDFGSARLLSNPMAFACTYVGTPYYVPPEIWENLPYN |
| NOV77c | KRVLHRDIKSQNIFLTQNGKVKLGDFGSARLLSNPMAFACTYVGTPYYVPPEIWENLPYN |
| NOV77d | KRVLHRDIKSQNIFLTQNGKVKLGDFGSARLLSNPMAFACTYVGTPYYVPPEIWENLPYN |
| NOV77a | NKSDIWSLGCILYELCTLKHPFQANSWKNLILKVCQGCISPLPSHYSYELQFLVKQMFKR |
| NOV77b | NKSDIWSLGCILYELCTLKHPFQANSWKNLILKVCQGCISPLPSHYSYELQFLVKQMFKR |
| NOV77c | NKSDIWSLGCILYELCTLKHPFQANSWKNLILKVCQGCISPLPSHYSYELQFLVKQMFKR |
| NOV77d | NKSDIWSLGCILYELCTLKHPFQANSWKNLILKVCQGCISPLPSHYSYELQFLVKQMFKR |
| NOV77a | NPSHRPSATTLLSRGIVARLVQKCLPPEIIMEYGEEVLEEIKNSKHNTPRKKSLLK---- |
| NOV77b | NPSHRPSATTLLSRGIVARLVQKCLPPEIIMEYGEEVLEEIKNSKHNTPRKKTNPSRIRI |
| NOV77c | NPSHRPSATTLLSRGIVARLVQKCLPPEIIMEYGEEVLEEIKNSKHNTPRKKQ------- |
| NOV77d | NPSHRPSATTLLSRGIVARLVQKCLPPEIIMEYGEEVLEEIKNSKHNTPRKKQ------- |
| NOV77a | ---------QEEEQDRKGSHTDLESINENLVESALRRVNRKS-GNKSVHLRKASSPNLHR |
| NOV77b | ALGNEASTVQEEEQDRKGSHTDLESINENLVESALRRVNREEKGNKSVHLRKASSPNLHR |
| NOV77c | ----------EEEQDRKGSHTDLESINENLVESALRRVNREEKGNKSVHLRKASSPNLHR |
| NOV77d | ----------EEEQDRKGSHTDLESINENLVESALRRVNREEKGNKSVHLRKASSPNLHR |
| NOV77a | RQWEKNVPNTALTALENASILTSSLTAEDDRGGGSVIKYSKNTTRKQWLKETPDTLLNIL |
| NOV77b | RQWEKNVPNTALTALENASILTSSLTAEDDRGG-SVIKYSKNTTRKQWLKETPDTLLNIL |
| NOV77c | RQWEKNVPNTALTALENASILTSSLTABDDRGG-SVIKYSKNTTRKQWLKETPDTLLNIL |
| NOV77d | RQWEKNVPNTALTALENASILTSSLTAEDDRG---------------------------- |
| NOV77a | KNADLSLAFQTYTIYRPGSEGFLKGPLSEETEASDSVDGGHDSVILDPERLEPGLDEEDT |
| NOV77b | KNADLSLAFQTYTIYRPGSEGFLKGPLSEETEASDSVDGGHDSVILDPERLEPGLDEEDT |
| NOV77c | KNADLSLAFQTYTIYRPGSEGFLKGPLSEETEASDSVDGGHDSVILDPERLEPGLDEEDT |
| NOV77d | ------------------SEGFLKGPLSEETEASDSVE-------------------EDT |
| NOV77a | DFEEEDDNPDWVSELKKRAGWQGLCD- |
| NOV77b | DFEEEDDNPDWVSELKKRAGWQGLCDR |
| NOV77c | DFEEEDDNPDWVSELKKRAGWQGLCDR |
| NOV77d | DFEEEDDNPDWVSELKKRAGWQGLCDR |
| NOV77a | (SEQ ID NO: 1098) |
| NOV77b | (SEQ ID NO: 1100) |
| NOV77c | (SEQ ID NO: 1102) |
| NOV77d | (SEQ ID NO: 1104) |

Further analysis of the NOV77a protein yielded the following properties shown in Table 77C.

TABLE 77C
Protein Sequence Properties NOV77a

| | |
|---|---|
| SignalP analysis: | No Known Signal Sequence Indicated |

PSORT II analysis:

| | |
|---|---|
| PSG: | a new signal peptide prediction method |
| | N-region: length 8; pos.chg 1; neg.chg 2 |
| | H-region: length 3; peak value 0.00 |
| | PSG score: −4.40 |
| GvH: | von Heijne's method for signal seq. recognition |
| | GvH score (threshold: −2.1): −8.46 |
| | possible cleavage site: between 16 and 17 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 4.61 (at 5) |
| | ALOM score: 4.61 (number of TMSs: 0) |

MITDISC: discrimination of mitochondrial targeting seq

| | | | |
|---|---|---|---|
| R content: | 0 | Hyd Moment(75): | 8.48 |
| Hyd Moment(95): | 7.98 | G content: | 0 |
| D/E content: | 2 | S/T content: | 0 |

| | Score: −6.50 |
|---|---|
| Gavel: | prediction of cleavage sites for mitochondrial preseq |
| | cleavage site motif not found |

NUCDISC: discrimination of nuclear localization signals

pat4: HKKR (3) at 116
pat4: PRKK (4) at 286
pat7: PRKKSLL (5) at 286
bipartite: none
content of basic residues: 13.3%
NLS Score: 0.40

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 76.7 |
| COIL: | Lupas's algorithm to detect coiled-coil regions |
| | total: 0 residues |

Final Results (k = 9/23):

```
69.6%: nuclear
21.7%: cytoplasmic
  4.3%: mitochondrial
  4.3%: peroxisomal
```

>> prediction for CG93669-04 is nuc (k = 23)

A search of the NOV77a protein against the Geneseq database, a proprietary database that contains sequences published in patents and patent publication, yielded several homologous proteins shown in Table 77D.

TABLE 77D
Geneseq Results for NOV77a

| | | NOV77a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP60668 | Human serine/threonine protein | 1 . . . 489 | 480/506 (94%) | 0.0 |
| | kinase 55.66 - Homo sapiens, 506 | 1 . . . 505 | 484/506 (94%) | |
| | aa. [CN1360042-A, 24-JUL- | | | |
| | 2002] | | | |
| AAM78344 | Human protein SEQ ID NO 1006 - | 1 . . . 489 | 480/506 (94%) | 0.0 |
| | Homo sapiens, 506 aa. | 1 . . . 505 | 484/506 (94%) | |
| | [WO200157190-A2, 09-AUG- | | | |
| | 2001] | | | |
| ABB97224 | Novel human protein SEQ ID | 1 . . . 489 | 479/506 (94%) | 0.0 |
| | NO: 492 - Homo sapiens, 527 aa. | 22 . . . 526 | 483/506 (94%) | |
| | [WO200222660-A2, 21-MAR- | | | |
| | 2002] | | | |
| AAM79328 | Human protein SEQ ID NO 2974 - | 1 . . . 489 | 479/506 (94%) | 0.0 |
| | Homo sapiens, 527 aa. | 22 . . . 526 | 483/506 (94%) | |
| | [WO200157190-A2, 09-AUG- | | | |
| | 2001] | | | |
| AAE24136 | Human kinase (PKIN)-7 protein - | 1 . . . 489 | 479/506 (94%) | 0.0 |
| | Homo sapiens, 506 aa. | 1 . . . 505 | 483/506 (94%) | |
| | [WO200233099-A2, 25-APR- | | | |
| | 2002] | | | |

In a BLAST search of public sequence databases, the NOV77a protein was found to have homology to the proteins shown in the BLASTP data in Table 77E.

TABLE 77E
Public BLASTP Results for NOV77a

| | | NOV77a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8J023 | NIMA-related protein kinase 3 - | 1 . . . 489 | 479/493 (97%) | 0.0 |
| | Homo sapiens (Human), 489 aa. | 1 . . . 488 | 482/493 (97%) | |
| P51956 | Serine/threonine-protein kinase | 1 . . . 489 | 480/506 (94%) | 0.0 |
| | NEK3 (EC 2.7.1.37) (NimA- | 1 . . . 505 | 484/506 (94%) | |
| | related protein kinase 3) (HSPK | | | |
| | 36) - Homo sapiens (Human), 506 | | | |
| | aa. | | | |
| CAD34740 | Sequence 48 from Patent | 1 . . . 489 | 479/506 (94%) | 0.0 |
| | WO0222660 - Homo sapiens | 22 . . . 526 | 483/506 (94%) | |
| | (Human), 527 aa. | | | |
| Q9R0A5 | Serine/threonine-protein kinase | 1 . . . 484 | 368/501 (73%) | 0.0 |
| | NEK3 (EC 2.7.1.37) (NimA- | 1 . . . 498 | 413/501 (81%) | |
| | related protein kinase 3) - Mus | | | |
| | musculus (Mouse), 511 aa. | | | |
| Q99K72 | Similar to NIMA (never in mitosis | 1 . . . 484 | 367/499 (73%) | 0.0 |
| | gene a)-related expressed kinase 3 - | 1 . . . 496 | 411/499 (81%) | |
| | Mus musculus (Mouse), 509 aa. | | | |

PFam analysis indicates that the NOV77a protein contains the domains shown in the Table 77F.

TABLE 77F
Domain Analysis of NOV77a

| | | Identities/ | |
|---|---|---|---|
| | NOV77a Match | Similarities | Expect |
| Pfam Domain | Region | for the Matched Region | Value |
| pkinase | 4 . . . 254 | 97/297 (33%) | 5.4e−87 |
| | | 207/297 (70%) | |

### Example 78

The NOV78 clone was analyzed, and the nucleotide and encoded polypeptide sequences are shown in Table 78A.

TABLE 78A
NOV78 Sequence Analysis

| | | |
|---|---|---|
| NOV78a, CG94235-01 | SEQ ID NO: 1111 | 2856 bp |
| DNA Sequence | ORF Start: ATG at 28 | ORF Stop: TAG at 1294 |

GGGCGGCGCGGGGTCTGCGCTGGGGCCATGGCTCCGCCGCGCCGCTTCGTCCTGGAGCTTCCCGACTG
CACCCTGGCTCACTTCGCCCTAGGCGCCGACGCCCCCGGCGACGCAGACGCCCCCGACCCCCGCCTGG
CGGCGCTGCTGGGGCCCCCGGAGCGCAGCTACTCGCTGTGCGTGCCCGTGACCCCGGACGCCGGCTGC
GGGGCCCGGGTCCGGGCGGCGCGGCTGCACCAGCGCCTGCTGCACCAGCTGCGCCGCGGCCCCTTCCA
GCGGTGCCAGCTGCTCAGGCTGCTCTGCTACTGCCCGGGCGGCCAGGCCGGCGGCGCACAGCAAGGCT
TCCTGCTGCGCGACCCCCTGGATGACCCTGACACCCGGCAAGCGCTGCTCGAGCTGCTGGGCGCCTGT
CAGGAGGCACCACGCCCGCACTTGGGCGAGTTCGAGGCCGACCCGCGCGGCCAGCTGTGGCAGCGCCT
CTGGGAGGTGCAAGACGGCAGGCGGCTGCAGGTGGGCTGCGCACAGGTCGTGCCCGTCCCGGAGCCCC
CGCTGCACCCGGTGGTGCCAGACTTGCCCAGTTCCGTGGTCTTCCCGGACCGGGAAGCCGCCCGGGCC
GTTTTGGAGGAGTGTACCTCCTTTATTCCTGAAGCCCGGGCAGTGCTTGACCTGGTCGACCAGTGCCC
AAAACAGATCCAGAAAGGAAAGTTCCAGGTTGTTGCCATCGAAGGACTGGATGCCACGGGTGGTAAAA
CCACGGTGACCCAGTCAGTGGCAGATTCACTTAAGGCTGTCCTCTTAAAGTCACCACCCTCTTGCATT
GGCCAGTGGAGGAAGATCTTTGATGATGAACCAACTATCATTAGAAGAGCTTTTTACTCTTTGGGCAA
TTATATTGTGGCCTCCGAAATAGCTAAAGAATCTGCCAAATCTCCTGTGATTGTAGACAGGCACAGCA
CGGCCACCTATGCCATAGCCACTGAGGTGAGTGGGGGTCTCCAGCACCTGCCCCCAGCCCATCACCCT
GTGTACCAGTGGCCAGAGGACCTGCTCAAACCTGACCTTATCCTGCTGCTCACTGTGAGTCCTGAGGA
GAGGTTGCAGAGGCTGCAGGGCCGGGGCATGGAGAAGACCAGGGAAGAAGCAGAACTTGAGGCCAACA
GTGTGTTTCGTCAAAAGGTAGAAATGTCCTACCAGCGGATGGAGAATCCTGGCTGCCATGTGGTTGAT
GCCAGCCCCTCCAGAGAAAAGGTCCTGCAGACGGTATTAAGCCTAATCCAGAATAGTTTTAGTGAACC
GTAGTTACTCTGGCCAGGTGCCACGTCTAACTAGATTAGATGTTGTTTGAAACATCTACATCCACCAT
TTGTTATGCAGTGTTCCCAAATTTCTGTTCTACAAGCATGTTGTGTGGCAGAAAACTGGAGACCAGGC
ATCTTAATTTTACTTCAGCCATCGTACCCTCTTCTGACTGATGGACCCGTCATCACAAAGGTCCCTCT
CATCATGTTCCAGTGAGAGGCCAGCGATTGCTTTCTTCCTGGCATAGTAAACATTTTCTTGGAACATA
TGTTTCACTTAATCACTACCAAATATCTGGAAGACCTGTCTTACTCAGACAGCACCAGGTGTACAGAA
GCAGCAGACAAGATCTTCCAGATCAGCAGGGAGACCCCGGAGCCTCTGCTTCTCCTACACTGGCATGC
TGATGAGATCGTGACATGCCCACATTGGCTTCTTCCACATCTGGTTGCACTCGTCATGATGGGCTCGC
TGCATCTCCCTCAGTCCCAAATTCTAGAGCCAAGTGTTCCTGCAGAGGCTGTCTATGTGTCCTGGCTG
CCCAAGGACACTCCTGCAGAGCCATTTTTGGGTAAGGAACACTTACAAAGAAGGCATTGATCTTGTGT
CTGAGGCTCAGAGCCCTTTTGATAGGCTTCTGAGTCATATATAAAGACATTCAAGCCAAGATGCTCCA
ACTGCAAATATACCAACCTTCTCTGAATTATATTTTGCTTATTTATATTTCTTTTCTTTTTTTCTAAA
GTATGGCTCTGAATAGAATGCACATTTTCCATTGAACTGGATGCATTTCATTTAGCCAATCCAGTAAT
TTATTTATATTAATCTATACATAATATGTTTCCTCAGCATAGGAGCTATGATTCATTAATTAAAAGTG
GAGTCAAAACGCTAAATGCAATGTTTGTTGTGTATTTTCATTACACAAACTTAATTTGTCTTGTTAAA
TAAGTACAGTGGATCTTGGAGTGGGATTTCTTGGTAAATTATCTTGCACTTGAATGTCTCATGATTAC
ATATGAAATCGCTTTGACATATCTTTAGACAGAAAAAAGTAGCTGAGTGAGGGGGAAATTATAGAGCT
GTGTGACTTTAGGGAGTAGGTTGAACCAGGTGATTACCTAAAATTCCTTCCAGTTCAAAGGCAGATAA
ATCTGTAAATTATTTTATCCTATCTACCATTTCTTAAGAAGACATTACTCCAAAATAATTAAATTTAA
GGCTTTATCAGGTCTGCATATAGAATCTTAAATTCTAATAAAGTTTCATGTTAATGTCATAGGATTTT
TAAAAGAGCTATAGGTAATTTCTATATAATATGTGTATATTAAAATGTAATTGATTTCAGTTGAAAGT
ATTTTAAAGCTGATAAATAGCATTAGGGTTCTTTGCAATGTGGTATCTAGCTGTATTATTGGTTTTAT
TTACTTTAAACATTTTGAAAAGCTTATACTGGCAGCCTAGAAAAACAAACAATTAATGTATCTTTATG
TCCCTGGCACATGAATAAACTTTGCTGTGGTTTACTAATCTAAAAAAAAAAAAAAAAGGGCGGCCGCT

| | | | |
|---|---|---|---|
| NOV78a, CG94235-01 | SEQ ID NO: 1112 | 422 aa | MW at 46476.6kD |
| Protein Sequence | | | |

MAPPRRFVLELPDCTLAHFALGADAPGDADAPDPRLAALLGPPERSYSLCVPVTPDAGCGARVRAARL
HQRLLHQLRRGPFQRCQLLRLLCYCPGGQAGGAQQGFLLRDPLDDPDTRQALLELLGACQEAPRPHLG
EFEADPRGQLWQRLWEVQDGRRLQVGCAQVVPVPEPPLHPVVPDLPSSVVFPDREAARAVLEECTSFI
PEARAVLDLVDQCPKQIQKGKFQVVAIEGLDATGGKTTVTQSVADSLKAVLLKSPPSCIGQWRKIFDD
EPTIIRRAFYSLGNYIVASEIAKESAKSPVIVDRHSTATYAIATEVSGGLQHLPPAHHPVYQWPEDLL
KPDLILLLTVSPEERLQRLQGRGMEKTREEAELEANSVFRQKVEMSYQRMENPGCHVVDASPSREKVL
QTVLSLIQNSFSEP

| | | |
|---|---|---|
| NOV78b, 254647864 | SEQ ID NO: 1113 | 601 bp |
| DNA Sequence | ORF Start: at 2 | ORF Stop: |
| | | end of sequence |

CACCGGATCCATCGAAGGACTGGATGCCACGGGTGGTAAAACCACGGTGACCCAGTCAGTGGCAGATT
CACTTAAGGCTGTCCTCTTAAAGTCACCACCCTCTTGCATTGGCCAGTGGAGGAAGATCTTTGATGAT
GAACCAACTATCATTAGAAGAGCTTTTTACTCTTTGGGCAATTATATTGTGGCCTCCGAAATAGCTAA
AGAATCTGCCAAATCTCCTGTGATTGTAGACAGGTACTGGCACAGCACGGCCACCTATGCCATAGCCA
CTGAGGTGAGTGGGGGTCTCCAGCACCTGCCCCCAGCCCATCACCCTGTGTACCAGTGGCCAGAGGAC
CTGCTCAAACCTGACCTTATCCTGCTGCTCACTGTGAGTCCTGAGGAGAGGTTGCAGAGGCTGCAGGG
CCGGGGCATGGAGAAGACCAGGGAAGAAGCAGAACTTGAGGCCAACAGTGTGTTTCGTCAAAAGGTAG
AAATGTCCTACCAGCGGATGGAGAATCCTGGCTGCCATGTGGTTGATGCCAGCCCCTCCAGAGAAAAG
GTCCTGCAGACGGTATTAAGCCTAATCCAGAATAGTTTTAGTGAACCGGGTACCGGC

| | | | |
|---|---|---|---|
| NOV78b, 254647864 | SEQ ID NO: 1114 | 200 aa | MW at 22132.8kD |
| Protein Sequence | | | |

TGSIEGLDATGGKTTVTQSVADSLKAVLLKSPPSCIGQWRKIFDDEPTIIRRAFYSLGNYIVASEIAK
ESAKSPVIVDRYWHSTATYAIATEVSGGLQHLPPAHHPVYQWPEDLLKPDLILLLTVSPEERLQRLQG
RGMEKTREEABLEANSVFRQKVEMSYQRMENPGCHVVDASPSREKVLQTVLSLIQNSFSEPGTG

| | | |
|---|---|---|
| NOV78c, 254347797 | SEQ ID NO: 1115 | 601 bp |
| DNA Sequence | ORF Start: at 2 | ORF Stop: |
| | | end of sequence |

CACCGGATCCATCGAAGGACTGGATGCCACGGGTGGTAAAACCACGGTGACCCAGTCAGTGGCAGATT
CACTTAAGGCTGTCCTCTTAAAGTCACCACCCTCTTGCATTGGCCAGTGGAGGAAGATCTTTGATGAT
GAACCAACTATCATTAGAAGAGCTTTTTACTCTTTGGGCAATTATATTGTGGCCTCCGAAATAGCTAA
AGAATCTGCCAAATCTCCTGTGATTGTAGACAGGTACTGGCACAGCACGGCCACCTATGCCATAGCCA
CTGAGGTGAGTGGGGGTCTCCAGCACCTGCCCCCAGCCCATCACCCTGTGTACCAGTGGCCAGAGGAC
CTGCTCAAACCTGACCTTATCCTGCTGCTCACTGTGAGTCCTGAGGAGAGGTTGCAGAGGCTGCAGGG
CCGGGGCATGGAGAAGACCAGGGAAGAAGCAGAACTTGAGGCCAACAGTGTGTTTCGTCAAAAGGTAG
AAATGTCCTACCAGCGGATGGAGAATCCTGGCTGCCATGTGGTTGATGCCAGCCCCTCCAGAGAAAAG
GTCCTGCAGACGGTATTAAGCCTAATCCAGAATAGTTTTAGTGAACCGGGTACCGGC

| | | | |
|---|---|---|---|
| NOV78c, 254347797 | SEQ ID NO: 1116 | 200 aa | MW at 22132.8kD |
| Protein Sequence | | | |

TGSIEGLDATGGKTTVTQSVADSLKAVLLKSPPSCIGQWRKIFDDEPTIIRRAFYSLGNYIVASEIAK
ESAKSPVIVDRYNHSTATYAIATEVSGGLQHLPPAHHPVYQWPEDLLKPDLILLLTVSPEERLQRLQG
RGMEKTREEAELEANSVFRQKVEMSYQRNENPGCHVVDASPSREKVLQTVLSLIQNSFSEPGTG

| | | |
|---|---|---|
| NOV78d, CG94235-02 | SEQ ID NO: 1117 | 2331 bp |
| DNA Sequence | ORF Start: ATG at 16 | ORF Stop: TAG at 769 |

GTCTGCGCTGGGGCCATGGCTCCGCCGCGCCGCTTCGTCCTGGAGCTTCCTGACTGCACCCTGGCTCA
CTTCGCCCTAGGCGCCGTTTTGGAGGAGTGTACCTCCTTTATTCCTGAAGCCCGGGCAGTGCTTGACC
TGGTCGACCAGTGCCCAAAACAGATCCAGAAAGGAAAGTTCCAGGTTGTTGCCATCGAAGGACTGGAT
GCCACGGGTAAAACCACGGTGACCCAGTCAGCGGCAGATTCACTTAAGGCTGTCCTCTTAAAGTCACC
ACCCTCTTGCATTGGCCAGTGGAGGAAGATCTTTGATGATGAACCAACTATCATTAGAAGAGCTTTTT
ACTCTTTGGGCAATTATATTGTGGCCTCCGAAATAGCTAAAGAATCTGCCAAATCTCCTGTGATTGTA
GACAGGTACTGGCACAGCACGGCCACCTATGCCATAGCCACTGAGGTGAGTGGGGGTCTCCAGCACCT
GCCCCCAGCCCATCACCCTGTGTACCAGTGGCCAGAGGACCTGCTCAAACCTGACCTTATCCTGCTGC
TCACTGTGAGTCCTGAGGAGAGGTTGCAGAGGCTGCAGGGCCGGGGCATGGAGAAGACCAGGGAAGAA
GCAGAACTTGAGGCCAACAGTGTGTTTCGTCAAAAGGTAGAAATGTCCTACCAGCGGATGGAGAATCC
TGGCTGCCATGTGGTTGATGCCAGCCCCTCCAGAGAAAAGGTCCTGCAGACGGTATTAAGCCTAATCC
AGAATAGTTTTAGTGAACCGTAGTTACTCTGGCCAGGTGCCACGTCTAACTAGATTAGATGTTGTTTG
AAACATCTACATCCACCATTTGTTATGCAGTGTTCCCAAATTTCTGTTCTACAAGCATGTTGTGTGGC
AGAAAACTGGAGACCAGGCATCTTAAGTTTACTTCAGCCATCGTACCCTCTTCTGACTGATGGACCCG
TCATCACAAAGGTCCCTCTCATCATGTTCCAGTGAGAGGCCAGCGATTGCTTTCTTCCTGGCATAGTA
AACATTTTCTTGGAACATATGTTTCACTTAATCACTACCAAATATCTGGAAGACCTGTCTTACTCAGA
CAGCACCAGGTGTACAGAAGCAGCAGACAAGATCTTCCAGATCAGCAGGGAGACCCCGGAGCCTCTGC
TTCTCCTACACTGGCATGCTGATGAGATCGTGACATGCCCACATTGGCTTCTTCCACATCTGGTTGCA
CTCGTCATGATGGGCTCGCTGCATCTCCCTCAGTCCCAAATTCTAGAGCCAAGTGTTCCTGCAGAGGC
TGTCTATGTGTCCTGGCTGCCCAAGGACACTCCTGCAGAGCCATTTTTGGGTAAGGAACACTTACAAA
GAAGGCATTGATCTTGTGTCTGAGGCTCAGAGCCCTTTTGATAGGCTTCTGAGTCATATATAAAGACA
TTCAAGCCAAGATGCTCCAACTGCAAATATACCAACCTTCTCTGAATTATATTTTGCTTATTTATATT
TCTTTTCTTTTTTTCTAAAGTATGGCTCTGAATAGAATGCACATTTTCCATTGAACTGGATGCATTTC
ATTTAGCCAATCCAGTAATTTATTTATATTAATCTATACATAATATGTTTCCTCAGCATAGGAGCTAT
GATTCATTAATTAAAAGTGGAGTCAAAACGCTAAATGCAATGTTTGTTGTGTATTTTCATTACACAAA
CTTAATTTGTCTTGTTAAATAAGTACAGTGGATCTTGGAGTGGGATTTCTTGGTAAATTATCTTGCAC
TTGAATGTCTCATGATTACATATGAAATCGCTTTGACATATCTTTAGACAGAAAAAAGTAGCTGAGTG
AGGGGGAAATTATAGAGCTGTGTGACTTTAGGGAGTAGGTTGAACCAGGTGATTACCTAAAATTCCTT
CCAGTTCAAAGGCAGATAAATCTGTAAATTATTTTATCCTATCTACCATTTCTTAAGAAGACATTACT
CCAAAATAATTAAATTTAAGGCTTTATCAGGTCTGCATATAGAATCTTAAATTCTAATAAAGTTTCAT
GTTAATGTCATAGGATTTTTAAAAGAGCTATAGGTAATTTCTGTATAATATGTGTATATTAAAATGTA
ATTGATTTCAGTTGAAAGTATTTTAAAGCTGATAAATAGCATTAGGGTTCTTTGCAATGTGGTATCTA
GCTGTATTATTGGTTTTATTTACTTTAAACATTTTGAAAAGCTTATACTGGCAGCCTAGAAAAACAAA
CAATTAATGTATCTTTATGTCCCTGGCACATGAATAAACTTTGCTGTGGTTTACTAAAAAAAAAAAAA
AAAAAAAAGGGCGGCCGCT

| | | | |
|---|---|---|---|
| NOV78d, CG94235-02 | SEQ ID NO: 1118 | 251 aa | MW at 27980.8kD |
| Protein Sequence | | | |

MAPPRRFVLELPDCTLAHFALGAVLEECTSFIPEARAVLDLVDQCPKQIQKGKFQVVAIEGLDATGKT
TVTQSAADSLKAVLLKSPPSCIGQWRKIFDDEPTIIRRAFYSLGNYIVASEIAKESAKSPVIVDRYWH
STATYAIATEVSGGLQHLPPAHHPVYQWPEDLLKPDLILLLTVSPEERLQRLQGRGMEKTREEAELEA
NSVFRQKVEMSYQRMENPGCHVVDASPSREKVLQTVLSLIQNSFSEP

A ClustalW comparison of the above protein sequences yields the following sequence alignment shown in Table 78B.

TABLE 78B
Comparison of the NOV78 protein sequences.

| | |
|---|---|
| NOV78a | MAPPRRFVLELPDCTLAHFALGADAPGDADAPDPRLAALLGPPERSYSLCVPVTPDAGCG |
| NOV78b | ------------------------------------------------------------ |
| NOV78c | ------------------------------------------------------------ |
| NOV78d | ------------------------------------------------------------ |
| NOV78a | ARVRAARLHQRLLHQLRRGPFQRCQLLRLLCYCPGGQAGGAQQGFLLRDPLDDPDTRQAL |
| NOV78b | ------------------------------------------------------------ |
| NOV78c | ------------------------------------------------------------ |
| NOV78d | ------------------------------------------------------------ |
| NOV78a | LELLGACQEAPRPHLGEFEADPRGQLWQRLWEVQDGRRLQVGCAQVVPVPEPPLHPVVPD |
| NOV78b | ------------------------------------------------------------ |
| NOV78c | ------------------------------------------------------------ |
| NOV78d | --------------------------------------------------MAPPRRFVLE |
| NOV78a | LPSSVVFPDREAARAVLEECTSFIPEARAVLDLVDQCPKQIQKGKFQVVAIEGLDATGGK |
| NOV78b | -----------------------------------------------TGSIEGLDATGGK |
| NOV78c | -----------------------------------------------TGSIEGLDATGGK |
| NOV78d | LPDCTLA--HFALGAVLEECTSFIPEARAVLDLVDQCPKQIQKGKFQVVAIEGLDATG-K |
| NOV78a | TTVTQSVADSLKAVLLKSPPSCIGQWRKIFDDEPTIIRRAFYSLGNYIVASEIAKESAKS |
| NOV78b | TTVTQSVADSLKAVLLKSPPSCIGQWRKIFDDEPTIIRRAFYSLGNYIVASEIAKESAKS |
| NOV78c | TTVTQSVADSLKAVLLKSPPSCIGQWRKIFDDEPTIIRRAFYSLGNYIVASEIAKESAKS |
| NOV78d | TTVTQSAADSLKAVLLKSPPSCIGQWRKIFDDEPTIIRRAFYSLGNYIVASEIAKESAKS |
| NOV78a | PVIVDR--HSTATYAIATEVSGGLQHLPPAHHPVYQWPEDLLKPDLILLLTVSPEERLQR |
| NOV78b | PVIVDRYWHSTATYAIATEVSGGLQHLPPAHHPVYQWPEDLLKPDLILLLTVSPEERLQR |
| NOV78c | PVIVDRYWHSTATYAIATEVSGGLQHLPPAHHPVYQWPEDLLKPDLILLLTVSPEERLQR |
| NOV78d | PVIVDRYWHSTATYAIATEVSGGLQHLPPAHHPVYQWPEDLLKPDLILLLTVSPEERLQR |
| NOV78a | LQGRGMEKTREEAELEANSVFRQKVEMSYQRMENPGCHVVDASPSREKVLQTVLSLIQNS |
| NOV78b | LQGRGMEKTREEAELEANSVFRQKVEMSYQRMENPGCHVVDASPSREKVLQTVLSLIQNS |
| NOV78c | LQGRGMEKTREEAELEANSVFRQKVEMSYQRMENPGCHVVDASPSREKVLQTVLSLIQNS |
| NOV78d | LQGRGMEKTREEAELEANSVFRQKVEMSYQRMENPGCHVVDASPSREKVLQTVLSLIQNS |
| NOV78a | FSEP--- |
| NOV78b | FSEPGTG |
| NOV78c | FSEPGTG |
| NOV78d | FSEP--- |
| NOV78a | (SEQ ID NO: 1112) |
| NOV78b | (SEQ ID NO: 1114) |
| NOV78c | (SEQ ID NO: 1116) |
| NOV78d | (SEQ ID NO: 1118) |

Further analysis of the NOV78a protein yielded the following properties shown in Table 78C.

TABLE 78C
Protein Sequence Properties NOV78a

| | |
|---|---|
| SignalP analysis: | No Known Signal Sequence Indicated |

PSORT II analysis:

| | |
|---|---|
| PSG: | a new signal peptide prediction method |
| | N-region: length 10; pos.chg 2; neg.chg 1 |
| | H-region: length 2; peak value −1.05 |
| | PSG score: −5.45 |
| GvH: | von Heijne's method for signal seq. recognition |
| | GvH score (threshold: −2.1): −5.20 |
| | possible cleavage site: between 22 and 23 |

>>> Seems to have no N-terminal signal peptide

| | |
|---|---|
| ALOM: | Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 3.66 (at 7) |
| | ALOM score: 3.66 (number of TMSs: 0) |

MITDISC: discrimination of mitochondrial targeting seq

| | | | |
|---|---|---|---|
| R content: | 2 | Hyd Moment(75): | 11.11 |
| Hyd Moment(95): | 9.49 | G content: | 0 |
| D/E content: | 2 | S/T content: | 0 |

Score: −4.13

Gavel: prediction of cleavage sites for mitochondrial preseq

R-3 motif at 48 ERSY|S

NUCDISC: discrimination of nuclear localization signals

pat4: none
pat7: none
bipartite: none
content of basic residues: 10.9%
NLS Score: −0.47

KDEL: ER retention motif in the C-terminus: none
ER Membrane Retention Signals:

XXRR-like motif in the N-terminus: APPR

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

47.8%: cytoplasmic
26.1%: mitochondrial
17.4%: nuclear
8.7%: peroxisomal

>> prediction for CG94235-01 is cyt (k = 23)

A search of the NOV78a protein against the Geneseq database, a proprietary database that contains sequences published in patents and patent publication, yielded several homologous proteins shown in Table 78D.

TABLE 78D
Geneseq Results for NOV78a

| | | NOV78a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB57297 | Mouse ischaemic condition related | 199 . . . 384 | 131/193 (67%) | 3e−64 |
| | protein sequence SEQ ID NO: 834 - | 157 . . . 344 | 150/193 (76%) | |
| | Mus musculus, 431 aa. | | | |
| | [WO200188188-A2, 22-NOV- | | | |
| | 2001] | | | |
| AAB72201 | E. coli thymidylate kinase amino | 229 . . . 421 | 51/212 (24%) | 0.062 |
| | acid sequence - Escherichia coli, | 6 . . . 210 | 88/212 (41%) | |
| | 213 aa. [WO200111025-A2, 15- | | | |
| | FEB-2001] | | | |
| AAU34536 | E. coli cellular proliferation | 229 . . . 421 | 51/212 (24%) | 0.062 |
| | protein #117 - Escherichia coli, | 6 . . . 210 | 88/212 (41%) | |
| | 213 aa. [WO200170955-A2, 27- | | | |
| | SEP-2001] | | | |
| AAY28786 | E. coli thymidylate kinase-1 - | 229 . . . 421 | 51/212 (24%) | 0.062 |
| | Escherichia coli, 213 aa. | 6 . . . 210 | 88/212 (41%) | |
| | [WO9941404-A2, 19-AUG-1999] | | | |
| AAY23912 | Amino acid sequence of a heat | 183 . . . 277 | 26/97 (26%) | 0.71 |
| | shock protein - Mycobacterium | 199 . . . 293 | 49/97 (49%) | |
| | leprae, 537 aa. [WO9935270-A1, | | | |
| | 15-JUL-1999] | | | |

In a BLAST search of public sequence databases, the NOV78a protein was found to have homology to the proteins shown in the BLASTP data in Table 78E.

TABLE 78E
Public BLASTP Results for NOV78a

| | | NOV78a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9DC34 | 1200004E04Rik protein - Mus | 42 . . . 418 | 292/379 (77%) | e−168 |
| | musculus (Mouse), 395 aa. | 16 . . . 393 | 328/379 (86%) | |
| Q62316 | Thymidylate kinase homologue - | 199 . . . 384 | 131/193 (67%) | 1e−63 |
| | Mus musculus (Mouse), 431 aa. | 157 . . . 344 | 150/193 (76%) | |
| Q96AL8 | Hypothetical protein - Homo | 365 . . . 422 | 58/58 (100%) | 4e−25 |
| | sapiens (Human), 58 aa | 1 . . . 58 | 58/58 (100%) | |
| | (fragment). | | | |
| O60970 | TKRP1 - Leishmania major, 274 | 199 . . . 418 | 73/220 (33%) | 1e−21 |
| | aa. | 44 . . . 254 | 108/220 (48%) | |
| Q8P3Y6 | Thymidylate kinase (EC 2.7.4.9) | 228 . . . 415 | 53/204 (25%) | 2e−04 |
| | (dTMP kinase) - Xanthomonas | 11 . . . 196 | 89/204 (42%) | |
| | campestris (pv. campestris), 227 | | | |
| | aa. | | | |

PFam analysis indicates that the NOV78a protein contains the domains shown in the Table 78F.

TABLE 78F
Domain Analysis of NOV78a

| | NOV78a | Identities/ | |
|---|---|---|---|
| | Match | Similarities for | |
| Pfam Domain | Region | the Matched Region | Expect Value |
| Thymidylate_kin | 231 . . . 411 | 54/210 (26%) | 2.2e−07 |
| | | 131/210 (62%) | |

### Example 79

The NOV79 clone was analyzed, and the nucleotide and encoded polypeptide sequences are shown in Table 79A.

TABLE 79A
NOV79 Sequence Analysis

| | | |
|---|---|---|
| NOV79a, CG95175-01 | SEQ ID NO: 1119 | 3117 bp |
| DNA Sequence | ORF Start: ATG at 21 | ORF Stop: TGA at 3078 |

CCTCCCCAGTAGCTGGGACTATGGGAGCGTGCCACCATGCCTGGTTAATTTTTGTATTTTTAGTAGAG
ATGGGGTTTCACCATGTTGGCCAGGCTTGTCTTCCCCTCTCCTTAGTTATCCTCCTGGATTCCAAAGC
CTCCCAGGCCGAGCTGGGCTGGACTGCACTGCCAAGTAATGGGTGGGAGGAGATCAGCGGCGTGGATG
AACACGACCGTCCCATCCGCACGTACCAAGTGTGCAATGTGCTGGAGCCCAACCAGGACAACTGGCTG
CAGACTGGCTGGATAAGCCGTGGCCGCGGGCAGCGCATCTTCGTGGAACTGCAGTTCACACTCCGTGA
CTGCAGCAGCATCCCTGGCGCCGCGGGTACCTGCAAGGAGACCTTCAACGTCTACTACCTGGAAACTG
AGGCCGACCTGGGCCGTGGGCGTCCCCGCCTAGGCGGCAAAATCGACACGATCGCGGCGGACGAGAGC
TTCACGCAGGGCGACCTGGGTGAGCGCAAGATGAAGCTGAACACAGAGGTGCGCGAGATCGGACCGCT
CAGCCGGCGGGGTTTCCACCTGGCCTTTCAGGACGTGGGCGCATGCGTGGCGCTTGTCTCGGTGCGCG
TCTACTACAAGCAGTGCCGCGCCACCGTGCGGGGCCTGGCCACGTTCCCAGCCACCGCAGCCGAGAGC
GCCTTCTCCACACTGGTGGAAGTGGCCGGAACGTGCGTGGCGCACTCGGAAGGGGAGCCTGGCAGCCC
CCCACGCATGCACTGCGGCGCCGACGGCGAGTGGCTGGTGCCTGTGGGCCGCTGCAGCTGCAGCGCGG
GATTCCAGGAGCGTGGTGACTTCTGCGAAGGTATCTGTCCCCCAGGGTTTTACAAGGTGTCCCCGCGG
CGGCCCCTCTGCTCACCGTGCCCAGAGCACAGCCGGGCCCTGGAAAACGCCTCCACCTTCTGCGTGTG
CCAGGACAGCTATGCGCGCTCACCCACCGACCCGCCCTCGGCTTCCTGCACCCGTCCGCCGTCGGCGC
CGCGGGACCTGCAGTACAGCCTGAGCCGCTCGCCGCTGGTGCTGCGACTGCGCTGGCTGCCGCCGGCC
GACTCGGGAGGCCGCTCGGACGTCACCTACTCGCTGCTGTGCCTGCGCTGCGGCCGCGAGGGCCCGGC
GGGCGCCTGCGAGGGGCCGCGCGTGGCCTTCCTACCGCGCCAGGCAGGGCTGCGGGAGCGAGCCGCCA
CGCTGCTGCACCTGCGGCCCGGCGCGCGCTACACCGTGCGCGTGGCCGCGCTCAACGGCGTCTCGGGC
CCGGCGGCCGCCGCGGGAACCACCTACGCGCAGGTCACCGTCTCCACCGGGCCCGGGGGTAAGGCCGT
CCGCGCCCCCCACCCCGAGGCCACCGCGCCTGCCGCCCCTGCGCCCTCTTGGGGCCGCCCCGTCGGTC
CTGCGGGATCAGCGCCCTGGGAGGAGGATGAGATCCGCAGGGACCGAGTGGAACCCCAGAGCGTGTCC
CTGTCGTGGCGGGAGCCCATCCCTGCCGGAGCCCCTGGGGCCAATGACACGGAGTACGAGATCCGATA
CTACGAGAAGGTGAGTGCGCAGAGTGAGCAGACTTACTCCATGGTGAAGACAGGGGCGCCCACAGTCA
CCGTGATTTTCCTCCCAGCTGCCTCAGGGTCCAGGGACCAGAGCCCCGCCATTGTCGTCACCGTAGTG
ACCATCTCGGCCCTCCTCGTCCTGGGCTCCGTGATGAGTGTGCTGGCCATTTGGAGGAGGAGGCCCTG
CAGCTATGGCAAAGGAGGAGGGGATGCCCATGATGAAGAGGAGCTGTATTTCCACTGTGAGTTGGCTG
GGAAAGTCCCAACACGTCGCACATTCCTGGACCCCCAGAGCTGTGGGGACCTGCTGCAGGCTGTGCAT
CTGTTCGCCAAGGAACTGGATGCGAAAAGCGTCACGCTGGAGAGGAGCCTTGGAGGAGGCAAGCTGGG
CGGGCGGTTTGGGGAGCTGTGCTGTGGCTGCTTGCAGCTCCCCGGTCGCCAGGAGCTGCTCGTAGCCG
TGCACATGCTGAGGGACAGCGCCTCCGACTCACAGAGGCTCGGCTTCCTGGCCGAGGCCCTCACGCTG
GGCCAGTTTGACCATAGCCACATCGTGCGGCTGGAGGGCGTTGTTACCCGAGGTAGGGGAAGCACCTT
GATGATTGTCACCGAGTACATGAGCCATGGGGCCCTGGACGGCTTCCTCAGGCAGCGGCACGAGGGGC
AGCTGGTGGCTGGGCAACTGATGGGGTTGCTGCCTGGGCTGGCATCAGCCATGAAGTATCTGTCAGAG
ATGGGCTACGTTCACCGGGGCCTGGCAGCTCGCCATGTGCTGGTCAGCAGCGACCTTGTCTGCAAGAT
CTCTGGCTTCGGGCGGGGCCCCCGGGACCGATCAGAGGCTGTCTACACCACTATGGTGAGGCTACAGA
GTGGCCGGAGCCCAGCGCTATGGGCCGCTCCCGAGACACTTCAGTTTGGCCACTTCAGCTCTGCCAGT
GACGTGTGGAGCTTCGGCATCATCATGTGGGAGGTGATGGCCTTTGGGGAGCGGCCTTACTGGGACAT
GTCTGGCCAAGACGTGGTGATCAAGGCTGTGGAGGATGGCTTCCGGCTGCCACCCCCCAGGAACTGTC
CTAACCTTCTGCACCGACTAATGCTCGACTGCTGGCAGAAGGACCCAGGTGAGCGGCCCAGGTTCTCC
CAGATCCACAGCATCCTGAGCAAGATGGTGCAGGACCCAGAGCCCCCCAAGTGTGCCCTGACTACCTG
TCCCAGGCCTCTGACCCGCAGGCCTCCCACTCCACTAGCCGACCGTGCCTTCTCCACCTTCCCCTCCT
TTGGCTCTGTGGGCGCGTGGCTGGAGGCCCTGGACCTGTGCCGCTACAAGGACAGCTTCGCGGCTGCT
GGCTATGGGAGCCTGGAGGCCGTGGCCGAGATGACTAGCCAGGACCTGGTGAGCCTAGGCATCTCTTT
GGCTGAACATCGAGAGGCCCTCCTCAGCGGGATCAGCGCCCTGCAGGCACGAGTGCTCCAGCTGCAGG
GCCAGGGGGTGCAGGTGTGAGTGGACCCCATTCTTCCAAGGCAGGACTCCGGTGGGG

| | | | |
|---|---|---|---|
| NOV79a, CG95175-01 | SEQ ID NO: 1120 | 1019 aa | MW at 110412.5kD |
| Protein Sequence | | | |

MGACHHAWLIFVFLVEMGFHHVGQACLPLSLVILLDSKASQAELGWTALPSNGWEEISGVDEHDRPIR
TYQVCNVLEPNQDNWLQTGWISRGRGQRIFVELQFTLRDCSSIPGAAGTCKETFNVYYLETEADLGRG
RPRLGGKIDTIAADESFTQGDLGERKMKLNTEVREIGPLSRRGFHLAFQDVGACVALVSVRVYYKQCR
ATVRGLATFPATAAESAFSTLVEVAGTCVAHSEGEPGSPPRMHCGADGEWLVPVGRCSCSAGFQERGD
FCEGICPPGFYKVSPRRPLCSPCPEHSRALENASTFCVCQDSYARSPTDPPSASCTRPPSAPRDLQYS
LSRSPLVLRLRWLPPADSGGRSDVTYSLLCLRCGREGPAGACEGPRVAFLPRQAGLRERAATLLHLRP
GARYTVRVAALNGVSGPAAAAGTTYAQVTVSTGPGGKAVRAPHPEATAPAAPAPSWGRPVGPAGSAPW
EEDEIRRDRVEPQSVSLSWREPIPAGAPGANDTEYEIRYYEKVSAQSEQTYSMVKTGAPTVTVIFLPA
ASGSRDQSPAIVVTVVTISALLVLGSVMSVLAIWRRRPCSYGKGGGDAHDEEELYFHCELAGKVPTRR
TFLDPQSCGDLLQAVHLFAKELDAKSVTLERSLGGGKLGGRFGELCCGCLQLPGRQELLVAVHMLRDS
ASDSQRLGFLABALTLGQFDHSHIVRLEGVVTRGRGSTLMIVTEYMSHGALDGFLRQRHEGQLVAGQL
MGLLPGLASAMKYLSEMGYVHRGLAARHVLVSSDLVCKISGFGRGPRDRSEAVYTTMVRLQSGRSPAL
WAAPETLQFGHFSSASDVWSFGIIMWEVMAFGERPYWDMSGQDVVIKAVEDGFRLPPPRNCPMLLHRL
MLDCWQKDPGERPRFSQIHSILSKMVODPEPPKCALTTCPRPLTRRPPTPLADRAFSTFPSFGSVGAW
LEALDLCRYKDSFAAAGYGSLEAVAEMTSQDLVSLGISLAEHREALLSGISALQARVLQLQGQGVQV

| | | |
|---|---|---|
| NOV79b, 275697118 | SEQ ID NO: 1121 | 547 bp |
| DNA Sequence | ORF Start: at 2 | ORF Stop: |
| | | end of sequence |

CACCGGATCCGTTATCCTCCTGGATTCCAAAGCCTCCCAGGCCGAGCTGGGCTGGACTGCACTGCCAA
GTAATGGGTGGGAGGAGATCAGCGGCGTGGATGAACACGACCGTCCCATCCGCACGTACCAAGTGTGC
AATGTGCTGGAGCCCAACCAGGACAACTGGCTGCAGACTGGCTGGATAAGCCGTGGCCGCGGGCAGCG
CATCTTCGTGGAACTGCAGTTCACACTCCGTGACTGCAGCAGCATCCCTGGCGCCGCGGGTACCTGCA
AGGAGACCTTCAACGTCTACTACCTGGAAACTGAGGCCGACCTGGGCCGTGGGCGTCCCCGCCTAGGC
GGCAGCCGGCCCCGCAAAATCGACACGATCGCGGCGGACGAGAGCTTCACGCAGGGCGACCTGGGTGA
GCGCAAGATGAAGCTGAACACAGAGGTGCGCGAGATCGGACCGCTCAGCCGGCGGGGTTTCCACCTGG
CCTTTCAGGACGTGGGCGCATGCGTGGCGCTTGTCTCGGTGCGCGTCTACTACAAGCAGTGCGTCGAC
GGC

| | | | |
|---|---|---|---|
| NOV79b, 275697118 | SEQ ID NO: 1122 | 182 aa | MW at 20253.6kD |
| Protein Sequence | | | |

TGSVILLDSKASQAELGWTALPSNGWEEISGVDEHDRPIRTYQVCNVLEPNQDNWLQTGWISRGRGQR
IFVELQFTLRDCSSIPGAAGTCKETFNVYYLETEADLGRGRPRLGGSRPRKIDTIAADESFTQGDLGE
RKMKLNTEVREIGPLSRRGFHLAFQDVGACVALVSVRVYYKQCVDG

| | | |
|---|---|---|
| NOV79c, 275697150 | SEQ ID NO: 1123 | 694 bp |
| DNA Sequence | ORF Start: at 2 | ORF Stop: |
| | | end of sequence |

CACCGGATCCCTCGTAGCCGTGCACATGCTGAGGGACAGCGCCTCCGACTCACAGAGGCTCGGCTTCC
TGGCCGAGGCCCTCACGCTGGGCCAGTTTGACCATAGCCACATCGTGCGGCTGGAGGGCGTTGTTACC
CGAGGAAGCACCTTGATGATTGTCACCGAGTACATGAGCCATGGGGCCCTGGACGGCTTCCTCAGGCG
GCACGAGGGGCAGCTGGTGGCTGGGCAACTGATGGGGTTGCTGCCTGGGCTGGCATCAGCCATGAAGT
ATCTGTCAGAGATGGGCTACGTTCACCGGGGCCTGGCAGCTCGCCATGTGCTGGTCAGCAGCGACCTT
GTCTGCAAGATCTCTGGCTTCGGGCGGGGCCCCCGGGACCGATCAGAGGCTGTCTACACCACTATGAG
TGGCCGGAGCCCAGCGCTATGGGCCGCTCCCGAGACACTTCAGTTTGGCCACTTCAGCTCTGCCAGTG
ACGTGTGGAGCTTCGGCATCATCATGTGGGAGGTGATGGCCTTTGGGGAGCGGCCTTACTGGGACATG
TCTGGCCAAGACGTGATCAAGGCTGTGGAGGATGGCTTCCGGCTGCCACCCCCCAGGAACTGTCCTAA
CCTTCTGCACCGACTAATGCTCGACTGCTGGCAGAAGGACCCAGGTGAGCGGCCCAGGTTCTCCCAGA
TCCACGTCGACGGC

| | | | |
|---|---|---|---|
| NOV79c, 275697150 | SEQ ID NO: 1124 | 231 aa | MW at25557.0kD |
| Protein Sequence | | | |

TGSLVAVHNLRDSASDSQRLGFLAEALTLGQFDHSHIVRLEGVVTRGSTLMIVTEYMSHGALDGFLRR
HEGQLVAGQLMGLLPGLASAMKYLSEMGYVHRGLAARHVLVSSDLVCKISGFGRGPRDRSEAVYTTMS
GRSPALWAAPETLQFGHFSSASDVWSFGIIMWEVMAFGERPYWDMSGQDVIKAVEDGFRLPPPRNCPN
LLHRLMLDCWQKDPGERPRFSQIHVDG

A ClustalW comparison of the above protein sequences yields the following sequence alignment shown in Table 79B.

TABLE 79B
Comparison of the NOV79 protein sequences.

| | |
|---|---|
| NOV79a | MGACHHAWLIFVFLVEMGFHHVGQACLPLSLVILLDSKASQAELGWTALPSNGWEEISGV |
| NOV79b | ----------------------------TSGVILLDSKASQAELGWTALPSNGWEEISGV |
| NOV79c | ------------------------------------------------------------ |
| NOV79a | DEHDRPIRTYQVCNVLEPNQDNWLQTGWISRGRGQRIFVELQFTLRDCSSIPGAAGTCKE |
| NOV79b | DEHDRPIRTYQVCNVLEPNQDNWLQTGWISRGRGQRIFVELQFTLRDCSSIPGAAGTCKE |
| NOV79c | ------------------------------------------------------------ |
| NOV79a | TFNVYYLETEADLGRGRPRLGG----KIDTIAADESFTQGDLGERKMKLNTEVREIGPLS |
| NOV79b | TFNVYYLETEADLGRGRPRLGGSRPRKIDTIAADESFTQGDLGERKMKLNTEVREIGPLS |
| NOV79c | ------------------------------------------------------------ |
| NOV79a | RRGFHLAFQDVGACVALVSVRVYYKQCRATVRGLATFPATAAESAFSTLVEVAGTCVAHS |
| NOV79b | RRGFHLAFQDVGACVALVSVRVYYKQCVDG------------------------------ |
| NOV79c | ------------------------------------------------------------ |
| NOV79a | EGEPGSPPRMHCGADGEWLVPVGRCSCSAGFQERGDFCEGICPPGFYKVSPRRPLCSPCP |
| NOV79b | ------------------------------------------------------------ |
| NOV79c | ------------------------------------------------------------ |
| NOV79a | EHSRALENASTFCVCQDSYARSPTDPPSASCTRPPSAPRDLQYSLSRSPLVLRLRWLPPA |
| NOV79b | ------------------------------------------------------------ |
| NOV79c | ------------------------------------------------------------ |
| NOV79a | DSGGRSDVTYSLLCLRCGREGPAGACEGPRVAFLPRQAGLRERAATLLHLRPGARYTVRV |
| NOV79b | ------------------------------------------------------------ |
| NOV79c | ------------------------------------------------------------ |
| NOV79a | AALNGVSGPAAAAGTTYAQVTVSTGPGGKAVRAPHPEATAPAAPAPSWGRPVGPAGSAPW |
| NOV79b | ------------------------------------------------------------ |
| NOV79c | ------------------------------------------------------------ |
| NOV79a | EEDEIRRDRVEPQSVSLSWREPIPAGAPGANDTEYEIRYYEKVSAQSEQTYSMVKTGAPT |
| NOV79b | ------------------------------------------------------------ |
| NOV79c | ------------------------------------------------------------ |
| NOV79a | VTVIFLPAASGSRDQSPAIVVTVVTISALLVLGSVMSVLAIWRRRPCSYGKGGGDAHDEE |
| NOV79b | ------------------------------------------------------------ |
| NOV79c | ------------------------------------------------------------ |
| NOV79a | ELYFHCELAGKVPTRRTFLDPQSCGDLLQAVHLFAKELDAKSVTLERSLGGGKLGGRFGE |
| NOV79b | ------------------------------------------------------------ |
| NOV79c | ------------------------------------------------------------ |
| NOV79a | LCCGCLQLPGRQELLVAVHMLRDSASDSQRLGFLAEALTLGQFDHSHIVRLEGVVTRGRG |
| NOV79b | ------------------------------------------------------------ |
| NOV79c | -----------TGSLVAVHMLRDSASDSQRLGFLAEALTLGQFDHSHIVRLEGVVTRGRG |
| NOV79a | STLMIVTEYMSHGALDGFLRQRHEGQLVAGQLMGLLPGLASAMKYLSEMGYVHRGLAARH |
| NOV79b | ------------------------------------------------------------ |
| NOV79c | STLMIVTEYMSHGALDGFLRR-HEGQLVAGQLMGLLPGLASAMKYLSEMGYVHRGLAARH |
| NOV79a | VLVSSDLVCKISGFGRGPRDRSEAVYTTMVRLQSGRSPALWAAPETLQFGHFSSASDVWS |
| NOV79b | ------------------------------------------------------------ |
| NOV79c | VLVSSDLVCKISGFGRGPRDRSEAVYTTMS----GRSPALWAAPETLQFGHFSSASDVWS |
| NOV79a | FGIIMWEVMAFGERPYWDMSGQDVVIKAVEDGFRLPPPRNCPNLLHRLMLDCWQKDPGER |
| NOV79b | ------------------------------------------------------------ |
| NOV79c | FGIIMWEVMAFGERPYWDMSGQ-DVIKAVEDGFRLPPPRNCPNLLHRLMLDCWQKDPGER |
| NOV79a | PRFSQIHSILSKMVQDPEPPKCALTTCPRPLTRRPPTPLADRAFSTFPSFGSVAGWLEAL |
| NOV79b | ------------------------------------------------------------ |
| NOV79c | PRFSQIHVDG |
| NOV79a | DLCRYKDSFAAAGYGSLEAVAEMTSQDLVSLGISLAEHREALLSGISALQARVLQLQGQG |
| NOV79b | ------------------------------------------------------------ |
| NOV79c | ------------------------------------------------------------ |
| NOV79a | VQV |
| NOV79b | --- |
| NOV79c | --- |
| NOV79a | (SEQ ID NO: 1120) |
| NOV79b | (SEQ ID NO: 1122) |
| NOV79c | (SEQ ID NO: 1124) |

Further analysis of the NOV79a protein yielded the following properties shown in Table 79C.

TABLE 79C
Protein Sequence Properties NOV79a

| | |
|---|---|
| SignalP analysis: | Cleavage site between residues 43 and 44 |

PSORT II analysis:

| | |
|---|---|
| PSG: | a new signal peptide prediction method |
| | N-region: length 0; pos.chg 0; neg.chg 0 |
| | H-region: length 15; peak value 12.14 |
| | PSG score: 7.74 |
| GvH: | von Heijne's method for signal seq. recognition |
| | GvH score (threshold: −2.1): −2.44 |
| | possible cleavage site: between 41 and 42 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 3 | |
| | Number of TMS(s) for threshold 0.5: 1 | |
| | INTEGRAL | Likelihood = −9.08 Transmembrane 555-571 |
| | PERIPHERAL | Likelihood = 0.85 (at 743) |

| | ALOM score: −9.08 (number of TMSs: 1) |
|---|---|
| MTOP: | Prediction of membrane topology (Hartmann et al.) |
| | Center position for calculation: 562 |
| | Charge difference: 3.5 C(3.5) − N(0.0) |
| | C > N: C-terminal side will be inside |

>>>Caution: Inconsistent mtop result with signal peptide
>>>membrane topology: type 1b (cytoplasmic tail 555 to 1019)
MITDISC: discrimination of mitochondrial targeting seq

| | | | |
|---|---|---|---|
| R content: | 0 | Hyd Moment(75): | 3.60 |
| Hyd Moment(95): | 3.84 | G content: | 1 |
| D/E content: | 1 | S/T content: | 0 |

Score: −6.09

Gavel: prediction of cleavage sites for mitochondrial preseq

cleavage site motif not found

NUCDISC: discrimination of nuclear localization signals

pat4: RRRP (4) at 579
pat7: none
bipartite: none
content of basic residues: 10.1%
NLS Score: −0.22

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 565
LL at 623
LL at 670
LL at 751
LL at 880
LL at 998

checking 63 PROSITE DNA binding motifs:

Leucine zipper pattern (PS00029): *** found ***

LGGRFGELCCGCLQLPGRQELL at 650
LVSLGISLAEHREALLSGISAL at 984

none
checking 71 PROSITE ribosomal protein motifs: none
checking 33 PROSITE prokaryotic DNA binding motifs: none
NNCN: Reinhardt's method for Cytoplasmic/Nuclear discrimination

Prediction: cytoplasmic
Reliability: 89

COIL: Lupas's algorithm to detect coiled-coil regions

total: 0 residues

Final Results (k = 9/23):

```
34.8%: nuclear
21.7%: mitochondrial
21.7%: cytoplasmic
  8.7%: vesicles of secretory system
  4.3%: vacuolar
  4.3%: peroxisomal
  4.3%: endoplasmic reticulum
```

>> prediction for CG95175-01 is nuc (k = 23)

A search of the NOV79a protein against the Geneseq database, a proprietary database that contains sequences published in patents and patent publication, yielded several homologous proteins shown in Table 79D.

TABLE 79D
Geneseq Results for NOV79a

| | | NOV79a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM47209 | Human NOV3 protein - Homo | 7 . . . 1019 | 933/1054 (88%) | 0.0 |
| | sapiens, 1000 aa. | 9 . . . 1000 | 936/1054 (88%) | |
| | [WO200174851-A2, 11-OCT- | | | |
| | 2001] | | | |
| AAU76874 | Human EphA full length kinase - | 33 . . . 1019 | 925/1027 (90%) | 0.0 |
| | Homo sapiens, 974 aa. | 1 . . . 974 | 926/1027 (90%) | |
| | [WO200208253-A2, 31-JAN- | | | |
| | 2002] | | | |
| ABB98843 | Human NEPHA - Homo | 24 . . . 1019 | 931/1046 (89%) | 0.0 |
| | sapiens, 1008 aa. | 18 . . . 1008 | 932/1046 (89%) | |
| | [WO200283735-A1, 24-OCT- | | | |
| | 2002] | | | |
| AAE19158 | Human kinase polypeptide | 24 . . . 1019 | 913/1048 (87%) | 0.0 |
| | (PKIN-16) - Homo sapiens, | 18 . . . 1009 | 916/1048 (87%) | |
| | 1009 aa. [WO200208399-A2, | | | |
| | 31-JAN-2002] | | | |
| AAU03553 | Human protein kinase #53 - | 24 . . . 1019 | 913/1048 (87%) | 0.0 |
| | Homo sapiens, 1009 aa. | 18 . . . 1009 | 916/1048 (87%) | |
| | [WO200138503-A2, 31-MAY- | | | |
| | 2001] | | | |

In a BLAST search of public sequence databases, the NOV79a protein was found to have homology to the proteins shown in the BLASTP data in Table 79E.

TABLE 79E
Public BLASTP Results for NOV79a

| | | NOV79a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD13085 | Sequence 5 from Patent | 7 . . . 1019 | 933/1054 (88%) | 0.0 |
| | WO0174851 - Homo sapiens | 9 . . . 1000 | 936/1054 (88%) | |
| | (Human), 1000 aa. | | | |
| CAD23752 | Sequence 1 from Patent | 33 . . . 1019 | 925/1027 (90%) | 0.0 |
| | WO0208253 - Homo sapiens | 1 . . . 974 | 926/1027 (90%) | |
| | (Human), 974 aa (fragment). | | | |
| CAD23753 | Sequence 3 from Patent | 33 . . . 1019 | 930/1071 (86%) | 0.0 |
| | WO0208253 - Homo sapiens | 1 . . . 1022 | 931/1071 (86%) | |
| | (Human), 1022 aa (fragment). | | | |
| CAD23754 | Sequence 5 from Patent | 33 . . . 653 | 575/661 (86%) | 0.0 |
| | WO0208253 - Homo sapiens | 1 . . . 623 | 576/661 (86%) | |
| | (Human), 647 aa (fragment). | | | |
| CAD23755 | Sequence 7 from Patent | 33 . . . 653 | 580/705 (82%) | 0.0 |
| | WO0208253 - Homo sapiens | 1 . . . 671 | 581/705 (82%) | |
| | (Human), 695 aa (fragment). | | | |

PFam analysis indicates that the NOV79a protein contains the domains shown in the Table 79F.

TABLE 79F
Domain Analysis of NOV79a

| | | Identities/ | |
|---|---|---|---|
| | NOV79a Match | Similarities for | |
| Pfam Domain | Region | the Matched Region | Expect Value |
| EPH_lbd | 32 . . . 203 | 115/177 (65%) | 2.6e−119 |
| | | 156/177 (88%) | |
| fn3 | 331 . . . 426 | 25/98 (26%) | 2e−06 |
| | | 66/98 (67%) | |
| pkinase | 639 . . . 906 | 78/309 (25%) | 1.7e−37 |
| | | 186/309 (60%) | |
| SAM | 942 . . . 1006 | 27/68 (40%) | 2.9e−15 |
| | | 50/68 (74%) | |

### Example 80

The NOV80 clone was analyzed, and the nucleotide and encoded polypeptide sequences are shown in Table 80A.

TABLE 80A
NOV80 Sequence Analysis

| | | |
|---|---|---|
| NOV80a, CG99638-01 | SEQ ID NO: 1125 | 2131 bp |
| DNA Sequence | ORF Start: ATG at 93 | ORF Stop: TGA at 2088 |

CTAAATGAAGAGCGCTTGGGACCTGAACAACCAGCAGCGATACCCAGGTACAAAGGACCTCCAGACCA
GAGCCAGCCAGCAGCAAAAAGAGCATGGAGCTGAGGAGTACAGCAGCCCCCAGAGCTGAGGGCTACAG
CAACGTGGGCTTCCAGAATGAAGAAAACTTTCTTGAGAACGAGAACACATCAGGAAACAACTCAATAA
GAAGCAGAGCTGTGCAAAGCAGGGAGCACACAAACACCAAACAGGATGAAGAACAGGTCACAGTTGAG
CAGGATTCTCCAAGAAACAGAGAACACATGGAGGATGATGATGAGGAGATGCAACAAAAAGGGTGTTT
GGAAAGGAGGTATGACACAGTATGTGGTTTCTGTAGGAAACACAAAACAACTCTTCGGCACATCATCT
GGGGCATTTTATTAGCAGGTTATCTGGTTATGGTGATTTCGGCCTGTGTGCTGAACTTTCACAGAGCC
CTTCCTCTTTTTGTGATCACCGTGGCTGCCATCTTCTTTGTTGTCTGGGATCACCTGATGGCCAAATA
CGAACATCGAATTGATGAGATGCTGTCTCCTGGCAGAAGGCTTCTAAACAGCCATTGGTTCTGGCTGA
AGTGGGTGATCTGGAGCTCCCTGGTCCTAGCAGTTATTTTCTGGTTGGCCTTTGACACTGCCAAATTG
GGTCAACAGCAGCTGGTGTCCTTCGGTGGGCTCATAATGTACATTGTCCTGTTATTTCTATTTTCCAA
GTACCCAACCAGAGTTTACTGGAGACCTGTCTTATGGGGAATCGGGCTACAGTTTCTTCTTGGGCTCT
TGATTCTAAGGACTGACCCTGGATTTATAGCTTTTGATTGGTTGGGCAGACTAGTTCAGGTCCTGCCG
ATCGTGGTTTTCTTCAGCACTGTGATGTCCATGCTGTACTACCCTGGACTGATGCAGTGGATTATTAG
AAAGGTTGGATGGATCATGCTAGTTACTACGGGATCATCTCCTATTGAATCTGTAGTTGCTTCTGGGC
ATATATTTGTTGGACAAACGGAGTCTCCACTGCTGGTCCGACCATATTTACCTTACATCACCAAGTCT
GAACTCCACGCCATCATGACCGCCGGGTTCTCTACCATTGCTAGGTGCGTGCTAGGTGCATACATTTC
TTTTGGGGTTCCATCCTCCCACTTGTTAACAGCGTCAGTTATGTCAGCACCTGCGTCATTGGCTGCTG
CTAAACTCTTTTGGCCTGAGACAGAAAAACCTAAAATAACCCTCAAGAATGCCATGAAAATGGAAAGT
GGTGATTCAGGGAATCTTCTAGAAGCTGCAACACAGGGAGCATCCTCCTCCATCTCCCTGGTGGCCAA
CATCGCTGTGAATCTGATTGCCTTCCTGGCCCTGCTGTCTTTTATGAATTCAGCCCTGTCCTGGTTTG
GAAACATGTTTGACTACCCACAGCTGAGTTTTGAGCTAATCTGCTCCTACATCTTCATGCCCTTTTCC
TTCATGATGGGAGTGGAATGGCAGGACAGCTTTATGGTTGCCAGACTCATAGGTTATAAGACCTTCTT
CAATGAATTTGTGGCTTATGAGCACCTCTCAAAATGGATCCACTTGAGGAAAGAAGGTGGACCCAAAT
TTGTAAACGGTGTGCAGCAATATATATCAATTCGTTCTGAGATAATCGCCACTTACGCTCTCTGTGGT
TTTGCCAATATCGGGTCCCTAGGAATCGTGATCGGCGGACTCACATCCATGGCTCCTTCCAGAAAGCG
TGATATCGCCTCGGGGGCAGTGAGAGCTCTGATTGCGGGGACCGTGGCCTGCTTCATGACAGCCTGCA
TCGCAGGCATACTCTCCAGCACTCCTGTGGACATCAACTGCCATCACGTTTTAGAGAATGCCTTCAAC
TCCACTTTCCCTGGAAACACAACCAAGGTGATAGCTTGTTGCCAAAGTCTGTTGAGCAGCACTGTTGC
CAAGGGTCCTGGTGAAGTCATCCCAGGAGGAAACCACAGTCTGTATTCTTTGAAGGGCTGCTGCACAT
TGTTGAATCCATCGACCTTTAACTGCAATGGGATCTCTAATACATTTTGAGGTCAGCCACTTCTCCAG
TGGAACTCTGAAGTACAGATGCT

| | | | |
|---|---|---|---|
| NOV80a, CG99638-01 | SEQ ID NO: 1126 | 665 aa | MW at 73873.1kD |
| Protein Sequence | | | |

MELRSTAAPRABGYSNVGFQNEENFLENBNTSGNNSIRSRAVQSREHTNTKQDEEQVTVEQDSPRNRE
HMEDDDEEMQQKGCLERRYDTVCGFCRKHKTTLRHIIWGILLAGYLVMVISACVLNFHRALPLFVITV
AAIFFVVWDHLMAKYEHRIDEMLSPGRRLLNSHWFWLKWVIWSSLVLAVIFWLAFDTAKLGQQQLVSF
GGLIMYIVLLFLFSKYPTRVYWRPVLWGIGLQFLLGLLILRTDPGFIAFDWLGRLVQVLPIVVFFSTV
MSMLYYPGLMQWIIRKVGWIMLVTTGSSPIESVVASGHIFVGQTESPLLVRPYLPYITKSELHAIMTA
GFSTIAGSVLGAYISFGVPSSHLLTASVMSAPASLAAAKLFWPETEKPKITLKNAMKMESGDSGNLLE
AATQGASSSISLVANIAVNLIAFLALLSFMNSALSWFGNNFDYPQLSFELICSYIFMPFSFMMGVEWQ
DSFMVARLIGYKTFFNEFVAYEHLSKWIHLRKEGGPKFVNGVQQYISIRSEIIATYALCGFANIGSLG
IVIGGLTSMAPSRKRDIASGAVRALIAGTVACFMTACIAGILSSTPVDINCHHVLENAFNSTFPGNTT
KVIACCQSLLSSTVAKGPGEVIPGGNHSLYSLKGCCTLLNPSTFNCNGISNTF

Further analysis of the NOV80a protein yielded the following properties shown in Table 80B.

TABLE 80B
Protein Sequence Properties NOV80a

| | |
|---|---|
| SignalP analysis: | No Known Signal Sequence Indicated |

PSORT II analysis:

| | |
|---|---|
| PSG: | a new signal peptide prediction method |
| | N-region: length 10; pos.chg 2; neg.chg 1 |
| | H-region: length 1; peak value −3.60 |
| | PSG score: −8.00 |
| GvH: | von Heijne's method for signal seq. recognition |
| | GvH score (threshold: −2.1): −9.75 |
| | possible cleavage site: between 13 and 14 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 9 | | |
| | INTEGRAL | Likelihood = −7.96 | Transmembrane 107-123 |
| | INTEGRAL | Likelihood = −9.08 | Transmembrane 128-144 |
| | INTEGRAL | Likelihood = −5.84 | Transmembrane 175-191 |
| | INTEGRAL | Likelihood = −8.97 | Transmembrane 201-217 |
| | INTEGRAL | Likelihood = −4.51 | Transmembrane 228-244 |
| | INTEGRAL | Likelihood = −4.35 | Transmembrane 259-275 |
| | INTEGRAL | Likelihood = −6.58 | Transmembrane 418-434 |
| | INTEGRAL | Likelihood = −1.59 | Transmembrane 534-550 |
| | INTEGRAL | Likelihood = −4.94 | Transmembrane 569-585 |

| | |
|---|---|
| PERIPHERAL | Likelihood = 0.85 (at 454) |
| ALOM score: | −9.08 (number of TMSs: 9) |

| | |
|---|---|
| MTOP: | Prediction of membrane topology (Hartmann et al.) |
| | Center position for calculation: 114 |
| | Charge difference: −3.5 C(1.5) − N(5.0) |
| | N >= C: N-terminal side will be inside |

>>> membrane topology: type 3a
MITDISC: discrimination of mitochondrial targeting seq

| | | | |
|---|---|---|---|
| R content: | 2 | Hyd Moment(75): | 4.42 |
| Hyd Moment(95): | 1.72 | G content: | 0 |
| D/E content: | 2 | S/T content: | 2 |

Score: −5.36

Gavel: prediction of cleavage sites for mitochondrial preseq

cleavage site motif not found

NUCDISC: discrimination of nuclear localization signals

pat4: RKHK (3) at 95
pat7: PSRKRDI (5) at 555
bipartite: none
content of basic residues: 7.5%
NLS Score: 0.15

KDEL: ER retention motif in the C-terminus: none
ER Membrane Retention Signals:

XXRR-like motif in the N-terminus: ELRS

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |

COIL: Lupas's algorithm to detect coiled-coil regions

total: 0 residues

Final Results (k = 9/23):

77.8%: endoplasmic reticulum
11.1%: vesicles of secretory system
11.1%: nuclear

>> prediction for CG99638-01 is end (k = 9)

A search of the NOV80a protein against the Geneseq database, a proprietary database that contains sequences published in patents and patent publication, yielded several homologous proteins shown in Table 80C.

TABLE 80C
Geneseq Results for NOV80a

| | | NOV80a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE21150 | Human 52991 protein - Homo | 1 . . . 665 | 661/665 (99%) | 0.0 |
| | sapiens, 665 aa. [WO200218439- | 1 . . . 665 | 662/665 (99%) | |
| | A2, 07-MAR-2002] | | | |
| ABP69062 | Human polypeptide SEQ ID NO | 1 . . . 665 | 662/691 (95%) | 0.0 |
| | 1109 - Homo sapiens, 691 aa. | 1 . . . 691 | 663/691 (95%) | |
| | [WO200270539-A2, 12-SEP- | | | |
| | 2002] | | | |
| AAE22915 | Human transporter and ion | 1 . . . 665 | 661/691 (95%) | 0.0 |
| | channel (TRICH) 14 - Homo | 1 . . . 691 | 662/691 (95%) | |
| | sapiens, 691 aa. [WO200222684- | | | |
| | A2, 21-MAR-2002] | | | |
| AAW49106 | Rat jejunal concentrative | 93 . . . 652 | 261/588 (44%) | e−139 |
| | nucleoside transporter 1 (CNT1) - | 74 . . . 643 | 372/588 (62%) | |
| | Rattus sp, 648 aa. [WO9835990- | | | |
| | A1, 20-AUG-1998] | | | |
| AAW70260 | Human concentrative nucleoside | 93 . . . 652 | 261/589 (44%) | e−138 |
| | transporter 1 hCNT1c - Homo | 74 . . . 643 | 369/589 (62%) | |
| | sapiens, 649 aa. [WO9835990- | | | |
| | A1, 20-AUG-1998] | | | |

In a BLAST search of public sequence databases, the NOV80a protein was found to have homology to the proteins shown in the BLASTP data in Table 80D.

TABLE 80D
Public BLASTP Results for NOV80a

| | | NOV80a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD42381 | Sequence 1 from Patent | 1 . . . 665 | 661/665 (99%) | 0.0 |
| | WO0218439 - Homo sapiens | 1 . . . 665 | 662/665 (99%) | |
| | (Human), 665 aa. | | | |
| Q9HAS3 | Concentrative Na+-nucleoside | 1 . . . 665 | 662/691 (95%) | 0.0 |
| | cotransporter hCNT3 - Homo | 1 . . . 691 | 663/691 (95%) | |
| | sapiens (Human), 691 aa. | | | |
| Q9ERH8 | Concentrative Na+-nucleoside | 1 . . . 663 | 514/689 (74%) | 0.0 |
| | cotransporter mCNT3 - Mus | 19 . . . 701 | 584/689 (84%) | |
| | musculus (Mouse), 703 aa. | | | |
| Q8BWE2 | Solute carrier family 28 - Mus | 1 . . . 663 | 513/689 (74%) | 0.0 |
| | musculus (Mouse), 703 aa. | 19 . . . 701 | 583/689 (84%) | |
| Q91VD7 | Solute carrier family 28 (Sodium- | 1 . . . 663 | 511/689 (74%) | 0.0 |
| | coupled nucleoside transporter), | 19 . . . 701 | 584/689 (84%) | |
| | member 3 (Concentrative Na+- | | | |
| | nucleoside cotransporter) - Mus | | | |
| | musculus (Mouse), 703 aa. | | | |

PFam analysis indicates that the NOV80a protein contains the domains shown in the Table 80E.

TABLE 80E
Domain Analysis of NOV80a

| | | Identities/ | |
|---|---|---|---|
| | NOV80a | Similarities for | |
| Pfam Domain | Match Region | the Matched Region | Expect Value |
| NrfD | 97 . . . 357 | 67/387 (17%) | 0.23 |
| | | 179/387 (46%) | |
| Nucleoside_tra2 | 198 . . . 587 | 175/427 (41%) | 1.1e−157 |
| | | 312/427 (73%) | |

### Example 81

The NOV81 clone was analyzed, and the nucleotide and encoded polypeptide sequences are shown in Table 81A.

TABLE 81A
NOV81 Sequence Analysis

| | | |
|---|---|---|
| NOV81a, CG99650-01 | SEQ ID NO: 1127 | 1258 bp |
| DNA Sequence | ORF Start: ATG at 83 | ORF Stop: TGA at 1160 |

TGTTACCTGGAGACCCTCTGAGCTCTCACCTGCTACTTCIGCCGCTGCTTCTGCACAGAGCCCGGGCG
AGGACCCCTCCAGGATGCAGGTCCCGAACAGCACCGGCCCGGACAACGCGACGCTGCAGATGCTGCGG
AACCCGGCGATCGCGGTGGCCCTGCCCGTGGTGTACTCGCTGGTGGCGGCGGTCAGCATCCCGGGCAA
CCTCTTCTCTCTGTGGGTGCTGTGCCGGCGCATGGGGCCCAGATCCCCGTCGGTCATCTTCATGATCA
ACCTGAGCGTCACGGACCTGATGCTGGCCAGCGTGTTGCCTTTCCAAATCTACTACCATTGCAACCGC
CACCACTGGGTATTCGGGGTGCTGCTTTGCAACGTGGTGACCGTGGCCTTTTACGCAAACATGTATTC
CAGCATCCTCACCATGACCTGTATCAGCGTGGAGCGCTTCCTGGGGGTCCTGTACCCGCTCAGCTCCA
AGCGCTGGCGCCGCCGTCGTTACGCGGTGGCCGCGTGTGCAGGGACCTGGCTGCTGCTCCTGACCGCC
CTGTCCCCGCTGGCGCGCACCGATCTCACCTACCCGGTGCACGCCCTGGGCATCATCACCTGCTTCGA
CGTCCTCAAGTGGACGATGCTCCCCAGCGTGGCCATGTGGGCCGTGTTCCTCTTCACCATCTTCATCC
TGCTGTTCCTCATCCCGTTCGTGATCACCGTGGCTTGTTACACGGCCACCATCCTCAAGCTGTTGCGC
ACGGAGGAGGCGCACGGCCGGGAGCAGCGGAGGCGCGCGGTGGGCCTGGCCGCGGTGGTCTTGCTGGC
CTTTGTCACCTGCTTCGCCCCCAACAACTTCGTGCTCCTGGCGCACATCGTGAGCCGCCTGTTCTACG
GCAAGAGCTACTACCACGTGTACAAAGTCACGCTGTGTCTCAGCTGCCTCAACAACTGTCTGGACCCG
TTTGTTTATTACTTTGCGTCCCGGGAATTCCAGCTGCGCCTGCGGGAATATTTGGGCTGCCGCCGGGT
GCCCAGAGACACCCTGGACACGCGCCGCGAGAGCCTCTTCTCCGCCAGGACCACGTCCGTGCGCTCCG
AGGCCGGTGCGCACCCTGAAGGGATGGAGGGAGCCACCAGGCCCGGCCTCCAGAGGCAGGAGAGTGTG
TTCTGAGTCCCGGGGGCGCAGCTTGGAGAGCCGGGGGCGCAGCTTGGAGATCCAGGGGCGCATGGAGA
GGCCACGGTGCCAGAGGTTCAGGGAGAACAGCTG

| | | | |
|---|---|---|---|
| NOV81a, CG99650-01 | SEQ ID NO: 1128 | 359 aa | MW at 40620.4kD |
| Protein Sequence | | | |

MQVPNSTGPDNATLQMLRNPAIAVALPVVYSLVAAVSIPGNLFSLWVLCRRNGPRSPSVIFMINLSVT
DLMLASVLPFQIYYHCNRHHWVFGVLLCNVVTVAFYANMYSSILTMTCISVERFLGVLYPLSSKRWRR
RRYAVAACAGTWLLLLTALSPLARTDLTYPVHALGIITCFDVLKWTMLPSVAMWAVFLFTIFILLFLI
PFVITVACYTATILKLLRTEEAHGREQRRRAVGLAAVVLLAFVTCFAPNNFVLLAHIVSRLFYGKSYY
HVYKVTLCLSCLNNCLDPFVYYFASREFQLRLREYLGCRRVPRDTLDTRRESLFSARTTSVRSEAGAH
PEGMEGATRPGLQRQESVF

| | | |
|---|---|---|
| NOV81b, SNP13382525 | SEQ ID NO: 1129 | 1258 bp |
| of CG99650-01, | ORF Start: ATG at 83 | ORF Stop: TGA at 1160 |
| DNA Sequence | SNP Pos: 910 | SNP Change: A to G |

TGTTACCTGGAGACCCTCTGAGCTCTCACCTGCTACTTCTGCCGCTGCTTCTGCACAGAGCCCGGGCG
AGGACCCCTCCAGGATGCAGGTCCCGAACAGCACCGGCCCGGACAACGCGACGCTGCAGATGCTGCGG
AACCCGGCGATCGCGGTGGCCCTGCCCGTGGTGTACTCGCTGGTGGCGGCGGTCAGCATCCCGGGCAA
CCTCTTCTCTCTGTGGGTGCTGTGCCGGCGCATGGGGCCCAGATCCCCGTCGGTCATCTTCATGATCA
ACCTGAGCGTCACGGACCTGATGCTGGCCAGCGTGTTGCCTTTCCAAATCTACTACCATTGCAACCGC
CACCACTGGGTATTCGGGGTGCTGCTTTGCAACGTGGTGACCGTGGCCTTTTACGCAAACATGTATTC
CAGCATCCTCACCATGACCTGTATCAGCGTGGAGCGCTTCCTGGGGGTCCTGTACCCGCTCAGCTCCA
AGCGCTGGCGCCGCCGTCGTTACGCGGTGGCCGCGTGTGCAGGGACCTGGCTGCTGCTCCTGACCGCC
CTGTCCCCGCTGGCGCGCACCGATCTCACCTACCCGGTGCACGCCCTGGGCATCATCACCTGCTTCGA
CGTCCTCAAGTGGACGATGCTCCCCAGCGTGGCCATGTGGGCCGTGTTCCTCTTCACCATCTTCATCC
TGCTGTTCCTCATCCCGTTCGTGATCACCGTGGCTTGTTACACGGCCACCATCCTCAAGCTGTTGCGC
ACGGAGGAGGCGCACGGCCGGGAGCAGCGGAGGCGCGCGGTGGGCCTGGCCGCGGTGGTCTTGCTGGC
CTTTGTCACCTGCTTCGCCCCCAACAACTTCGTGCTCCTGGCGCACATCGTGAGCCGCCTGTTCTACG
GCAAGAGCTACTACCACGTGTACAAGGTCACGCTGTGTCTCAGCTGCCTCAACAACTGTCTGGACCCG
TTTGTTTATTACTTTGCGTCCCGGGAATTCCAGCTGCGCCTGCGGGAATATTTGGGCTGCCGCCGGGT
GCCCAGAGACACCCTGGACACGCGCCGCGAGAGCCTCTTCTCCGCCAGGACCACGTCCGTGCGCTCCG
AGGCCGGTGCGCACCCTGAAGGGATGGAGGGAGCCACCAGGCCCGGCCTCCAGAGGCAGGAGAGTGTG
TTCTGAGTCCCGGGGGCGCAGCTTGGAGAGCCGGGGGCGCAGCTTGGAGATCCAGGGGCGCATGGAGA
GGCCACGGTGCCAGAGGTTCAGGGAGAACAGCTG

| | | | |
|---|---|---|---|
| NOV81b, SNP13382525 | SEQ ID NO: 1130 | 359 aa | MW at 40620.4kD |
| of CG99650-01, | SNP Pos: 276 | | SNP Change: Lys to Lys |
| Protein Sequence | | | |

MQVPNSTGPDNATLQMLRNPAIAVALPVVYSLVAAVSIPGNLFSLWVLCRRMGPRSPSVIFMINLSVT
DLMLASVLPFQIYYHCNRHHWVFGVLLCNVVTVAFYANNYSSILTMTCISVERFLGVLYPLSSKRWRR
RRYAVAACAGTWLLLLTALSPLARTDLTYPVHALGIITCFDVLKWTMLPSVAMWAVFLFTIFILLFLI
PFVITVACYTATILKLLRTEEAHGREQRRRAVGLAAVVLLAFVTCFAPNNFVLLAHIVSRLFYGKSYY
HVYKVTLCLSCLNNCLDPFVYYFASREFQLRLREYLGCRRVPRDTLDTRRESLFSARTTSVRSEAGAH
PEGMEGATRPGLQRQESVF

| | | |
|---|---|---|
| NOV81c, SNP13382526 | SEQ ID NO: 1131 | 1258 bp |
| of CG99650-01, | ORF Start: ATG at 83 | ORF Stop: TGA at 1160 |
| DNA Sequence | SNP Pos: 911 | SNP Change: G to C |

TGTTACCTGGAGACCCTCTGAGCTCTCACCTGCTACTTCTGCCGCTGCTTCTGCACAGAGCCCGGGCG
AGGACCCCTCCAGGATGCAGGTCCCGAACAGCACCGGCCCGGACAACGCGACGCTGCAGATGCTGCGG
AACCCGGCGATCGCGGTGGCCCTGCCCGTGGTGTACTCGCTGGTGGCGGCGGTCAGCATCCCGGGCAA
CCTCTTCTCTCTGTGGGTGCTGTGCCGGCGCATGGGGCCCAGATCCCCGTCGGTCATCTTCATGATCA
ACCTGAGCGTCACGGACCTGATGCTGGCCAGCGTGTTGCCTTTCCAAATCTACTACCATTGCAACCGC
CACCACTGGGTATTCGGGGTGCTGCTTTGCAACGTGGTGACCGTGGCCTTTTACGCAAACATGTATTC
CAGCATCCTCACCATGACCTGTATCAGCGTGGAGCGCTTCCTGGGGGTCCTGTACCCGCTCAGCTCCA
AGCGCTGGCGCCGCCGTCGTTACGCGGTGGCCGCGTGTGCAGGGACCTGGCTGCTGCTCCTGACCGCC
CTGTCCCCGCTGGCGCGCACCGATCTCACCTACCCGGTGCACGCCCTGGGCATCATCACCTGCTTCGA
CGTCCTCAAGTGGACGATGCTCCCCAGCGTGGCCATGTGGGCCGTGTTCCTCTTCACCATCTTCATCC
TGCTGTTCCTCATCCCGTTCGTGATCACCGTGGCTTGTTACACGGCCACCATCCTCAAGCTGTTGCGC
ACGGAGGAGGCGCACGGCCGGGAGCAGCGGAGGCGCGCGGTGGGCCTGGCCGCGGTGGTCTTGCTGGC
CTTTGTCACCTGCTTCGCCCCCAACAACTTCGTGCTCCTGGCGCACATCGTGAGCCGCCTGTTCTACG
GCAAGAGCTACTACCACGTGTACAAACTCACGCTGTGTCTCAGCTGCCTCAACAACTGTCTGGACCCG
TTTGTTTATTACTTTGCGTCCCGGGAATTCCAGCTGCGCCTGCGGGAATATTTGGGCTGCCGCCGGGT
GCCCAGAGACACCCTGGACACGCGCCGCGAGAGCCTCTTCTCCGCCAGGACCACGTCCGTGCGCTCCG
AGGCCGGTGCGCACCCTGAAGGGATGGAGGGAGCCACCAGGCCCGGCCTCCAGAGGCAGGAGAGTGTG
TTCTGAGTCCCGGGGGCGCAGCTTGGAGAGCCGGGGGCGCAGCTTGGAGATCCAGGGGCGCATGGAGA
GGCCACGGTGCCAGAGGTTCAGGGAGAACAGCTG

| | | | |
|---|---|---|---|
| NOV81c, SNP13382526 | SEQ ID NO: 1132 | 359 aa | MW at 40634.5kD |
| of CG99650-01, | SNP Pos: 277 | | SNP Change: Val to Leu |
| Protein Sequence | | | |

MQVPNSTGPDNATLQMLRNPAIAVALPVVYSLVAAVSIPGNLFSLWVLCRRNGPRSPSVIFMINLSVT
DLMLASVLPFQIYYHCNRHHWVFGVLLCNVVTVAFYANMYSSILTMTCISVBRFLGVLYPLSSKRWRR
RRYAVAACAGTWLLLLTALSPLARTDLTYPVHALGIITCFDVLKWTMLPSVAMWAVFLFTIFILLFLI
PFVITVACYTATILKLLRTEEAHGREQRRRAVGLAAVVLLAFVTCFAPNNFVLLAHIVSRLFYGKSYY
HVYKLTLCLSCLNNCLDPFVYYFASREFQLRLREYLGCRRVPRDTLDTRRESLFSARTTSVRSEAGAH
PEGMEGATRPGLQRQESVF

A ClustalW comparison of the above protein sequences yields the following sequence alignment shown in Table 81B.

TABLE 81B
Comparison of the NOV81 protein sequences.

| | |
|---|---|
| NOV79a | MGACHHAWLIFVFLVEMGFHHVGQACLPLSLVILLDSKASQAELGWTALPSNGWEEISGV |
| NOV79b | ----------------------------TGSVILLDSKASQAELGWTALPSNGWEEISGV |
| NOV79c | ------------------------------------------------------------ |
| NOV79a | DEHDRPIRTYQVCNVLEPNQDNWLQTGWISRGRGQRIFVELQFTLRDCSSIPGAAGTCKE |
| NOV79b | DEHDRPIRTYQVCNVLEPNQDNWLQTGWISRGRGQRIFVELQFTLRDCSSIPGAAGTCKE |
| NOV79c | ------------------------------------------------------------ |
| NOV79a | TFNVYYLETEADLGRGRPRLGG----KIDTIAADESFTQGDLGERKMKLNTEVREIGPLS |
| NOV79b | TFNVYYLETEADLGRGRPRLGGSRPRKIDTIAADESFTQGDLGERKMKLNTEVREIGPLS |
| NOV79c | ------------------------------------------------------------ |
| NOV79a | RRGFHLAFQDVGACVALVSVRVYYKQCRATVRGLATFPATAAESAFSTLVEVAGTCVAHS |
| NOV79b | RRGFHLAFQDVGACVALVSVRVYYKQCVDG------------------------------ |
| NOV79c | ------------------------------------------------------------ |
| NOV79a | EGEPGSPPRMHCGADGEWLVPVGRCSCSAGFQERGDFCEGICPPGFYKVSPRRPLCSPCP |
| NOV79b | ------------------------------------------------------------ |
| NOV79c | ------------------------------------------------------------ |
| NOV79a | EHSRALENASTFCVCQDSYARSPTDPPSASCTRPPSAPRDLQYSLSRSPLVLRLRWLPPA |
| NOV79b | ------------------------------------------------------------ |
| NOV79c | ------------------------------------------------------------ |
| NOV79a | DSGGRSDVTYSLLCLRCGREGPAGACEGPRVAFLPRQAGLRERAATLLHLRPGARYTVRV |
| NOV79b | ------------------------------------------------------------ |
| NOV79c | ------------------------------------------------------------ |
| NOV79a | AALNGVSGPAAAAGTTYAQVTVSTGPGGKAVRAPHPEATAPAAPAPSWGRPVGPAGSAPW |
| NOV79b | ------------------------------------------------------------ |
| NOV79c | ------------------------------------------------------------ |
| NOV79a | EEDEIRRDRVEPQSVSLSWREPIPAGAPGANDTEYEIRYYEKVSAQSEQTYSMVKTGAPT |
| NOV79b | ------------------------------------------------------------ |
| NOV79c | ------------------------------------------------------------ |
| NOV79a | VTVIFLPAASGSRDQSPAIVVTVVTISALLVLGSVMSVLAIWRRRPCSYGKGGGDAHDEE |
| NOV79b | ------------------------------------------------------------ |
| NOV79c | ------------------------------------------------------------ |
| NOV79a | ELYFHCELAGKVPTRRTFLDPQSCGDLLQAVHLFAKELDAKSVTLERSLGGGKLGGRFGE |
| NOV79b | ------------------------------------------------------------ |
| NOV79c | ------------------------------------------------------------ |
| NOV79a | LCCGCLQLPGRQELLVAVHMLRDSASDSQRLGFLAEALTLGQFDHSHIVRLEGVVTRGRG |
| NOV79b | ------------------------------------------------------------ |
| NOV79c | -----------TGSLVAVHMLRDSASDSQRLGFLAEALTLGQFDHSHIVRLEGVVTRG-- |
| NOV79a | STLMIVTEYMSHGALDGFLRQRHEGQLVAGQLMGLLPGLASAMKYLSEMGYVHRGLAARH |
| NOV79b | ------------------------------------------------------------ |
| NOV79c | STLMIVTEYMSHGALDGFLRR-HEGQLVAGQLMGLLPGLASAMKYLSEMGYVHRGLAARH |
| NOV79a | VLVSSDLVCKISGFGRGPRDRSEAVYTTMVRLQSGRSPALWAAPETLQFGHFSSASDVWS |
| NOV79b | ------------------------------------------------------------ |
| NOV79c | VLVSSDLVCKISGFGRGPRDRSEAVYTTMS----GRSPALWAAPETLQFGHFSSASDVWS |
| NOV79a | FGIIMWEVMAFGERPYWDMSGQDVVIKAVEDGFRLPPPRNCPNLLHRLMLDCWQKDPGER |
| NOV79b | ------------------------------------------------------------ |
| NOV79c | FGIIMWEVMAFGERPYWDMSGQ-DVIKAVEDGFRLPPPRNCPNLLHRLMLDCWQKDPGER |
| NOV79a | PRFSQIHSILSKMVQDPEPPKCALTTCPRPLTRRPPTPLADRAFSTFPSFGSVGAWLEAL |
| NOV79b | ------------------------------------------------------------ |
| NOV79c | PRFSQIHVDG-------------------------------------------------- |
| NOV79a | DLCRYKDSFAAAGYGSLEAVAEMTSQDLVSLGISLAEHREALLSGISALQARVLQLQGQG |
| NOV79b | ------------------------------------------------------------ |
| NOV79c | ------------------------------------------------------------ |
| NOV79a | VQV |
| NOV79b | --- |
| NOV79c | --- |
| NOV81a | (SEQ ID NO: 1128) |

Further analysis of the NOV81a protein yielded the following properties shown in Table 81C.

TABLE 81C
Protein Sequence Properties NOV81a

| | |
|---|---|
| SignalP analysis: | Cleavage site between residues 38 and 39 |

PSORT II analysis:

| | |
|---|---|
| PSG: | a new signal peptide prediction method |
| | N-region: length 10; pos.chg 0; neg.chg 1 |
| | H-region: length 7; peak value 0.00 |
| | PSG score: −4.40 |
| GvH: | von Heijne's method for signal seq. recognition |
| | GvH score (threshold: −2.1): −4.98 |
| | possible cleavage site: between 49 and 50 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 6 | | |
| | INTEGRAL | Likelihood = −7.11 | Transmembrane 22-38 |
| | INTEGRAL | Likelihood = −2.76 | Transmembrane 59-75 |
| | INTEGRAL | Likelihood = −3.50 | Transmembrane 89-105 |
| | INTEGRAL | Likelihood = −2.28 | Transmembrane 140-156 |
| | INTEGRAL | Likelihood = −14.01 | Transmembrane 192-208 |
| | INTEGRAL | Likelihood = −9.18 | Transmembrane 235-251 |

| | |
|---|---|
| PERIPHERAL | Likelihood = 1.22 (at 110) |

| | ALOM score: −14.01 (number of TMSs: 6) |
|---|---|
| MTOP: | Prediction of membrane topology (Hartmann et al.) |
| | Center position for calculation: 29 |
| | Charge difference: 3.0 C(3.0) − N(0.0) |
| | C > N: C-terminal side will be inside |

>>> membrane topology: type 3b
MITDISC: discrimination of mitochondrial targeting seq

| | | | |
|---|---|---|---|
| R content: | 4 | Hyd Moment(75): | 2.17 |
| Hyd Moment(95): | 4.19 | G content: | 3 |
| D/E content: | 2 | S/T content: | 10 |

| | Score: −2.58 |
|---|---|
| Gavel: | prediction of cleavage sites for mitochondrial preseq |
| | R-2 motif at 232 LRT\|EE |

NUCDISC: discrimination of nuclear localization signals

pat4: RRRR (5) at 135
pat7: none
bipartite: none
content of basic residues: 9.7%
NLS Score: −0.16

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: vacuolar
11.1%: Golgi
11.1%: vesicles of secretory system
11.1%: mitochondrial

>> prediction for CG99650-01 is end (k = 9)

A search of the NOV81a protein against the Geneseq database, a proprietary database that contains sequences published in patents and patent publication, yielded several homologous proteins shown in Table 81D.

TABLE 81D
Geneseq Results for NOV81a

| | | NOV81a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB82503 | Human TGR341 polypeptide - | 1 . . . 359 | 358/359 (99%) | 0.0 |
| | Homo sapiens, 359 aa. | 1 . . . 359 | 359/359 (99%) | |
| | [WO200277001-A2, 03-OCT- | | | |
| | 2002] | | | |
| ABG73502 | Human G protein coupled | 1 . . . 359 | 358/359 (99%) | 0.0 |
| | receptor HGPRBMY1 - Homo | 1 . . . 359 | 359/359 (99%) | |
| | sapiens, 359 aa. [WO200268591- | | | |
| | A2, 06-SEP-2002] | | | |
| ABP81677 | Human LS160435 receptor | 1 . . . 359 | 358/359 (99%) | 0.0 |
| | protein SEQ ID NO: 530 - Homo | 1 . . . 359 | 359/359 (99%) | |
| | sapiens, 359 aa. [WO200261087- | | | |
| | A2, 08-AUG-2002] | | | |
| ABG93784 | Human G protein-coupled | 1 . . . 359 | 358/359 (99%) | 0.0 |
| | receptor protein, nGPCR-5- | 1 . . . 359 | 359/359 (99%) | |
| | Homo sapiens, 359 aa. | | | |
| | [WO200264789-A1, 22-AUG- | | | |
| | 2002] | | | |
| AAB62285 | Human G-protein coupled | 1 . . . 359 | 358/359 (99%) | 0.0 |
| | receptor, PAUL - Homo sapiens, | 1 . . . 359 | 359/359 (99%) | |
| | 359 aa. [WO200125280-A1, 12- | | | |
| | APR-2001] | | | |

In a BLAST search of public sequence databases, the NOV81a protein was found to have homology to the proteins shown in the BLASTP data in Table 81E.

TABLE 81E
Public BLASTP Results for NOV81a

| | | NOV81a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH43610 | Hypothetical protein - Homo | 1 . . . 359 | 358/359 (99%) | 0.0 |
| | sapiens (Human), 359 aa. | 1 . . . 359 | 359/359 (99%) | |
| Q8NH25 | Seven transmembrane helix | 16 . . . 218 | 200/203 (98%) | e−112 |
| | receptor - Homo sapiens | 1 . . . 203 | 200/203 (98%) | |
| | (Human), 258 aa. | | | |
| P47749 | Proteinase activated receptor 1 | 15 . . . 313 | 111/300 (37%) | 4e−54 |
| | precursor (PAR-1) (Thrombin | 93 . . . 386 | 172/300 (57%) | |
| | receptor) - Xenopus laevis | | | |
| | (African clawed frog), 420 aa. | | | |
| A37912 | thrombin receptor precursor - | 22 . . . 311 | 106/292 (36%) | 5e−53 |
| | human, 425 aa. | 101 . . . 389 | 167/292 (56%) | |
| P56488 | Proteinase activated receptor 1 | 22 . . . 311 | 106/292 (36%) | 9e−53 |
| | precursor (PAR-1) (Thrombin | 101 . . . 389 | 166/292 (56%) | |
| | receptor) - Papio hamadryas | | | |
| | (Hamadryas baboon), 425 aa. | | | |

PFam analysis indicates that the NOV81a protein contains the domains shown in the Table 81F.

TABLE 81F
Domain Analysis of NOV81a

| | | Identities/ | |
|---|---|---|---|
| | NOV81a Match | Similarities for | |
| Pfam Domain | Region | the Matched Region | Expect Value |
| Acyl_transf_3 | 9 . . . 274 | 44/468 (9%) | 0.76 |
| | | 178/468 (38%) | |
| 7tm_1 | 40 . . . 293 | 76/276 (28%) | 5.3e−50 |
| | | 193/276 (70%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below: cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—hole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. CT values below 28 indicate high expression, CT values between 28 and 32 indicate moderate expression, and CT values between 32 and 35 indicate low expression. CT values above 35 reflect levels of expression that are too low to be reliably measured.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:
ca.=carcinoma,
*=established from metastasis,
met=metastasis,
s cell var=small cell variant,
non-s=non-sm=non-small,
squam=squamous,
pl. eff=pl effusion=pleural effusion,
glio=glioma,
astro=astrocytoma, and
neuro=neuroblastoma.
   General_Screening_Panel_V1.4, V1.5, V1.6 and 1.7

The plates for Panels 1.4, 1.5, 1.6 and 1.7 include 2 control wells (genomic DNA control and chemistry control) and 88 to 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, 1.6 and 1.7 are widely available through the American Type Culture Collection (ATCC), a repository for cultured cell lines, and were cultured using the conditions recommended by the ATCC. The normal tissues found on Panels 1.4, 1.5, 1.6 and 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3, and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel V 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following-tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel V 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

ARDAIS Prostate V 1.0

The plates for ARDAIS prostate 1.0 generally include 2 control wells and 68 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

ARDAIS Kidney V 1.0

The plates for ARDAIS kidney 1.0 generally include 2 control wells and 44 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

Panel 3D and 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1, 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24,48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 4M non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes 6 (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL-4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-lanti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:
AI=Autoimmunity
Syn=Synovial
Normal=No apparent disease
Rep22/Rep20=individual patients
RA=Rheumatoid arthritis
Backus=From Backus Hospital
OA=Osteoarthritis
(SS) (BA) (MF)=Individual patients
Adj=Adjacent tissue
Match control=adjacent tissues
−M=Male
−F=Female
COPD=Chronic obstructive pulmonary disease
   AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1beta). These treatments include: IL-1β (10 ng/ml), IL-1β, +TNF-α (50 ng/ml), IL-1β+Oncostatin (50 ng/ml) and PMA (100 ng/ml). The SW1353 cells were obtained from the ATCC (American Type Culture Collection) and were all cultured under standard recommended conditions. The SW1353 cells were plated at 3×10⁵ cells/ml (in DMEM medium-10% FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:
Patient 2 Diabetic Hispanic, overweight, not on insulin
Patient 7-9 Nondiabetic Caucasian and obese (BMI>30)
Patient 10 Diabetic Hispanic, overweight, on insulin
Patient 11 Nondiabetic African American and overweight
Patient 12 Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | |
|---|---|---|
| Donor 2 and 3 U | Mesenchymal | Undifferentiated Adipose |
| | Stem cells | |
| Donor 2 and 3 AM | Adipose | AdiposeMidway Differentiated |
| Donor 2 and 3 AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:
GO Adipose=Greater Omentum Adipose
SK=Skeletal Muscle
UT=Uterus
PL=Placenta
AD=Adipose Differentiated
AM=Adipose Midway Differentiated.
U=Undifferentiated Stem Cells
   Human Metabolic RTQ-PCR Panel

The plates for the Human Metabolic RTQ-PCR Panel include two control wells (genomic DNA control and chemistry control) and 211 cDNAs isolated from human tissues and cell lines with an emphasis on metabolic diseases. This panel is useful for establishing the tissue and cellular expression profiles for genes believed to play a role in the etiology and pathogenesis of obesity and/or diabetes and to confirm differential expression of such genes derived from other methods.

Metabolic tissues were obtained from patients enrolled in the CuraGen Gestational Diabetes study and from autopsy tissues from Type II diabetics and age, sex and race-matched control patients. One or more of the following were used to characterize the patients: body mass index [BMI=wt (kg)/ht (m²)], serum glucose, HgbA1c. Cell lines used in this panel are widely available through the American Type Culture Collection (ATCC), a repository for cultured cell lines. RNA from human Pancreatic Islets was also obtained.

In the Gestational Diabetes study, subjects are young (18-40 years), otherwise healthy women with and without gestational diabetes undergoing routine (elective) Caesarian section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted, and then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus), and subcutaneous adipose. Patient descriptions are as follows:
Patient 7 Non-diabetic Caucasian and obese
Patient 8 Non-diabetic Caucasian and obese
Patient 12 Diabetic Caucasian with unknown BMI and on insulin
Patient 13 Diabetic Caucasian, overweight, not on insulin
Patient 15 Diabetic Caucasian, obese, not on insulin
Patient 17 Diabetic Caucasian, normal weight, not on insulin
Patient 18 Diabetic Hispanic, obese, not on insulin
Patient 19 Non-diabetic Caucasian and normal weight
Patient 20 Diabetic Caucasian, overweight, and on insulin
Patient 21 Non-diabetic Caucasian and overweight
Patient 22 Diabetic Caucasian, normal weight, on insulin
Patient 23 Non-diabetic Caucasian and overweight
Patient 25 Diabetic Caucasian, normal weight, not on insulin
Patient 26 Diabetic Caucasian, obese, on insulin
Patient 27 Diabetic Caucasian, obese, on insulin

Total RNA was isolated from metabolic tissues of 12 Type II diabetic patients and 12 matched control patients included hypothalamus, liver, pancreas, small intestine, psoas muscle, diaphragm muscle, visceral adipose, and subcutaneous adipose. The diabetics and non-diabetics were matched for age, sex, ethnicity, and BMI where possible.

The panel also contains pancreatic islets from a 22 year old male patient (with a BMI of 35) obtained from the Diabetes Research Institute at the University of Miami School of Medicine. Islet tissue was processed to total RNA at CuraGen.

Cell lines used in this panel are widely available through the American Type Culture Collection (ATCC), a repository for cultured cell lines, and were cultured at an outside facility. The RNA was extracted at CuraGen according to CuraGen protocols. All samples were then processed at CuraGen to produce single stranded cDNA.

In the labels used to identify tissues in the Human Metabolic panel, the following abbreviations are used:
Pl=placenta
Go=greater omentum
Sk=skeletal muscle
Ut=uterus
CC=Caucasian
HI=Hispanic
AA=African American
AS=Asian
Diab=Type H diabetic
Norm=Non-diabetic
Overwt=Overweight; med BMI
Obese=Hi BMI
Low BM=20-25
Med BM=26-30
Hi BMI=Greater than 30
M=Male
#=Patient identifier
Vis.=Visceral
SubQ=Subcutaneous
   Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:
PSP=Progressive supranuclear palsy
Sub Nigra=Substantia nigra
Glob Palladus=Globus palladus
Temp Pole=Temporal pole
Cing Gyr=Cingulate gyrus
BA 4=Brodman Area 4
   Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:
AD=Alzheimer's disease brain; patient was demented and showed AD-like pathology upon autopsy
Control=Control brains; patient not demented, showing no neuropathology
Control (Path)=Control brains; pateint not demented but showing sever AD-like pathology
SupTemporal Ctx=Superior Temporal Cortex
Inf Temporal Ctx=Inferior Temporal Cortex

The expression of the gene was analyzed after normalization using scaling factor. The scaling factor is calculated from the Grand mean of CT values for a panel and the Well mean which is specific to the tissue. The Grand mean is the average CT value for all wells across all runs. For example, if a panel has 50 samples and has had 100 probe/primer sets run on it, the grand mean would be the average of these 5000 CT scores. The well mean is tissue-specific. On the above described panel there would be 50 different well means, each taking the average of the 100 CT values generated for each sample on the panel from the 100 probe/primer sets.

The asumption is that across a large number of genes, all samples should have the same CT value. If a well is lower than the average across a large number of genes, it is “scaled up” by that difference or the “scaling factor”.
Scaling Factor=Grand mean−Well mean
The new CT value for the well is:
Scaled CT value=Raw CT+Scaling Factor.
Statistical Analysis of CNS_Neurodegeneration_V1.0 Data

All data were analyzed by analysis of covariance (ANCOVA). As a covariate, the average CT value (or number of rounds of PCR until signal from the well was detected) was calculated for 1000 PCR runs on different genes. This number is therefore an estimate of total cDNA quantity and quality for each sample. When RTQ PCR is run for a given gene, CT values are therefore compared to these average values to correct for differences in well loading or original RNA quality. Stats were run on data from the temporal cortex, as this regions shows sever neurodegeneration in the mid to late stages of the disease, and because the largest number of samples were available for this region giving the most statistical power. Covariates for each well corresponding to Temporal Cortex samples are listed below. The well numbers (10-25) are listed under “Order” in the table of CT values given for each gene run. For this analysis, Controls and Control (Path) cases were grouped together as the intention was to find genes associated with dementia as opposed to amyloid deposition.

| | | |
|---|---|---|
| 10 | AD1 | 33.014 |
| 11 | AD2 | 32.309 |
| 12 | AD3 | 34.195 |
| 13 | AD4 | 32.689 |
| 14 | AD5 Inf | 30.829 |
| 15 | AD5 Sup | 31.519 |
| 16 | AD6 Inf | 31.517 |
| 17 | AD6 Sup | 31.415 |
| 18 | Con1 | 34.236 |
| 19 | Con2 | 32.352 |
| 20 | Con3 | 33.215 |
| 21 | Con4 | 33.661 |
| 22 | Con5 (Path) | 31.685 |
| 23 | Con6 (Path) | 32.187 |
| 24 | Con7 (Path) | 34.427 |
| 25 | Con8 (Path) | 32.238 |

Panel CNS_Neurodegeneration_V2.0

The plates for Panel CNS_Neurodegeneration_V2.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains sixteen brains from Alzheimer's disease (AD) patients, and twenty-nine brains from “Normal controls” who showed no evidence of dementia prior to death. The twenty-nine normal control brains are divided into two categories: Fourteen controls with no dementia and no Alzheimer's like pathology (Controls) and fifteen controls with no dementia but evidence of severe Alzheimer's like pathology, (specifically senile plaque load rated as level 3 on a scale of 0-3; 0=no evidence of plaques, 3=severe AD senile plaque load). Tissue from the temporal cotex (Broddmann Area 21) was selected for all samples from the Harvard Brain Tissue Resource Center; from the two sample from the Human Brain and Spinal Fluid Resource Center (samples 1 and 2) tissue from the inferior and superior temporal cortex was used; each sample on the panel represents a pool of inferior and superior temporal cortex from an individual patient. The temporal cortex was chosen as it shows a loss of neurons in the intermediate stages of the disease. Selection of a region which is affected in the early stages of Alzheimer's disease (e.g., hippocampus or entorhinal cortex) could potentially result in the examination of gene expression after vulnerable neurons are lost, and missing genes involved in the actual neurodegeneration process.

In the labels employed to identify tissues in the CNS_Neurodegeneration_V2.0 panel, the following abbreviations are used:
AD=Alzheimer's disease brain; patient was demented and showed AD-like pathology upon autopsy
Control=Control brains; patient not demented, showing no neuropathology
AH3=Control brains; pateint not demented but showing sever AD-like pathology
Inf & Sup Temp Ctx Pool=Pool of inferior and superior temporal cortex for a given individual

A. CG101340-01: Putative G Protein-Coupled Receptor 92

Expression of full-length physical clone CG101340-01 was assessed using the primer-probe set Gpcr41, described in Table AA. Results of the RTQ-PCR runs are shown in Tables AB and AC.

TABLE AA
Probe Name Gpcr41

| | | | Start | SEQ ID |
|---|---|---|---|---|
| Primers | Sequences | Length | Position | No |
| Forward | 5′-ggtggtgagcgtgtacatgtg-3′ | 21 | 240 | 1133 |
| Probe | TET-5′-cagcgacctgctcttcaccctctcg-3′- | 25 | 273 | 1134 |
| | TAMRA | | | |
| Reverse | 5′-gtaggagagacgaacgggca-3′ | 20 | 299 | 1135 |

TABLE AB
Panel 1.3D
Column A - Rel. Exp.(%) Gpcr41, Run 158144249

| Tissue Name | A | Tissue Name | A |
|---|---|---|---|
| Liver adenocarcinoma | 4.6 | Kidney (fetal) | 0.7 |
| Pancreas | 0.9 | Renal ca. 786-0 | 0.3 |
| Pancreatic ca. CAPAN 2 | 21.2 | Renal ca. A498 | 0.4 |
| Adrenal gland | 8.1 | Renal ca. RXF 393 | 0.7 |
| Thyroid | 13.0 | Renal ca. ACHN | 0.0 |
| Salivary gland | 6.2 | Renal ca. UO-31 | 0.0 |
| Pituitary gland | 5.5 | Renal ca. TK-10 | 0.0 |
| Brain (fetal) | 3.4 | Liver | 1.0 |
| Brain (whole) | 14.9 | Liver (fetal) | 8.4 |
| Brain (amygdala) | 39.2 | Liver ca. (hepatoblast) HepG2 | 3.6 |
| Brain (cerebellum) | 6.7 | Lung | 10.2 |
| Brain (hippocampus) | 100.0 | Lung (fetal) | 2.2 |
| Brain (substantia nigra) | 13.7 | Lung ca. (small cell) LX-1 | 21.2 |
| Brain (thalamus) | 21.0 | Lung ca. (small cell) NCI-H69 | 0.4 |
| Cerebral Cortex | 22.8 | Lung ca. (s. cell var.) SHP-77 | 0.5 |
| Spinal cord | 22.4 | Lung ca. (large cell)NCI-H460 | 0.0 |
| glio/astro U87-MG | 0.0 | Lung ca. (non-sm. cell) A549 | 1.5 |
| glio/astro U-118-MG | 0.4 | Lung ca. (non-s. cell) NCI-H23 | 0.3 |
| astrocytoma SW 1783 | 0.8 | Lung ca. (non-s. cell) HOP-62 | 3.4 |
| neuro*; met SK-N-AS | 1.0 | Lung ca. (non-s. cl) NCI-H522 | 0.0 |
| astrocytoma SF-539 | 2.9 | Lung ca. (squam.) SW 900 | 0.0 |
| astrocytoma SNB-75 | 0.5 | Lung ca. (squam.) NCI-H596 | 0.0 |
| glioma SNB-19 | 0.0 | Mammary gland | 7.2 |
| glioma U251 | 0.0 | Breast ca.* (pl. ef) MCF-7 | 0.4 |
| glioma SF-295 | 1.5 | Breast ca.* (pl. ef) | 11.8 |
| | | MDA-MB-231 | |
| Heart (Fetal) | 45.1 | Breast ca.* (pl. ef) T47D | 0.0 |
| Heart | 4.2 | Breast ca. BT-549 | 2.5 |
| Skeletal muscle (Fetal) | 36.6 | Breast ca. MDA-N | 0.5 |
| Skeletal muscle | 1.5 | Ovary | 29.5 |
| Bone marrow | 7.7 | Ovarian ca. OVCAR-3 | 0.3 |
| Thymus | 10.2 | Ovarian ca. OVCAR-4 | 0.0 |
| Spleen | 50.0 | Ovarian ca. OVCAR-5 | 12.6 |
| Lymph node | 17.9 | Ovarian ca. OVCAR-8 | 6.9 |
| Colorectal | 95.9 | Ovarian ca. IGROV-1 | 0.5 |
| Stomach | 18.6 | Ovarian ca. (ascites) SK-OV-3 | 0.4 |
| Small intestine | 25.5 | Uterus | 2.6 |
| Colon ca. SW480 | 21.9 | Placenta | 17.0 |
| Colon ca.* SW620 | 9.9 | Prostate | 1.9 |
| (SW480 met) | | | |
| Colon ca. HT29 | 1.7 | Prostate ca.* (bone met) PC-3 | 1.6 |
| Colon ca. HCT-116 | 9.9 | Testis | 15.3 |
| Colon ca. CaCo-2 | 1.0 | Melanoma Hs688(A).T | 0.0 |
| CC Well to Mod Diff | 72.7 | Melanoma* (met) Hs688(B).T | 0.0 |
| (ODO3866) | | | |
| Colon ca. HCC-2998 | 31.2 | Melanoma UACC-62 | 0.0 |
| Gastric ca. (liver met) | 51.4 | Melanoma M14 | 0.0 |
| NCI-N87 | | | |
| Bladder | 10.8 | Melanoma LOX IMVI | 1.4 |
| Trachea | 20.0 | Melanoma* (met) SK-MEL-5 | 1.2 |
| Kidney | 1.1 | Adipose | 16.7 |

TABLE AC
Panel 4D
Column A - Rel. Exp. (%) Gpcr41, Run 142878269
Column B - Rel. Exp. (%) Gpcr41, Run 144060624

| Tissue Name | A | B | Tissue Name | A | B |
|---|---|---|---|---|---|
| Secondary Th1 act | 19.6 | 72.7 | HUVEC IL-1beta | 0.0 | 0.0 |
| Secondary Th2 act | 20.2 | 76.8 | HUVEC IFN gamma | 0.0 | 1.8 |
| Secondary Tr1 act | 20.7 | 39.2 | HUVEC TNF alpha + IFN gamma | 0.0 | 0.0 |
| Secondary Th1 rest | 23.0 | 22.5 | HUVEC TNF alpha +IL4 | 0.0 | 1.3 |
| Secondary Th2 rest | 44.4 | 31.0 | HUVEC IL-11 | 0.6 | 0.2 |
| Secondary Tr1 rest | 20.9 | 18.8 | Lung Microvascular EC none | 0.7 | 1.7 |
| Primary Th1 act | 14.9 | 8.7 | Lung Microvascular EC TNF alpha + | 0.2 | 1.1 |
| | | | IL-1beta | | |
| Primary Th2 act | 33.7 | 17.4 | Microvascular Dermal EC none | 0.0 | 0.0 |
| Primary Tr1 act | 25.7 | 12.2 | Microsvasular Dermal EC TNFalpha | 0.0 | 0.0 |
| | | | + IL-1beta | | |
| Primary Th1 rest | 89.5 | 36.6 | Bronchial epithelium TNFalpha + | 25.5 | 18.4 |
| | | | IL1beta | | |
| Primary Th2 rest | 56.3 | 19.5 | Small airway epithelium none | 8.3 | 6.3 |
| Primary Tr1 rest | 64.2 | 35.8 | Small airway epithelium TNFalpha + | 71.7 | 29.9 |
| | | | IL-1beta | | |
| CD45RA CD4 | 4.6 | 18.6 | Coronery artery SMG rest | 0.0 | 0.0 |
| lymphocyte act | | | | | |
| CD45RO CD4 | 18.2 | 57.4 | Coronery artery SMC TNFalpha + | 0.0 | 0.0 |
| lymphocyte act | | | IL-1beta | | |
| CD8 lymphocyte act | 20.3 | 48.3 | Astrocytes rest | 0.2 | 0.0 |
| Secondary CD8 | 13.9 | 18.0 | Astrocytes TNFalpha + IL-1beta | 0.7 | 1.0 |
| lymphocyte rest | | | | | |
| Secondary CD8 | 17.7 | 14.2 | KU-812 (Basophil) rest | 18.3 | 34.2 |
| lymphocyte act | | | | | |
| CD4 lymphocyte none | 7.4 | 6.3 | KU-812 (Basophil) PMA/ionomycin | 68.3 | 71.2 |
| 2ry Th1/Th2/Tr1 anti- | 59.5 | 49.3 | CCD1106 (Keratinocytes) none | 17.2 | 21.9 |
| CD95 CH11 | | | | | |
| LAK cells rest | 23.5 | 10.2 | 93580 CCD1106 (Keratinocytes) | 100.0 | 43.5 |
| | | | TNFa and IFNg | | |
| LAK cells IL-2 | 30.4 | 20.4 | Liver cirrhosis | 2.7 | 2.8 |
| LAK cells IL-2 + IL-12 | 24.8 | 15.7 | Lupus kidney | 3.0 | 2.1 |
| LAK cells IL-2 + IFN | 45.1 | 16.3 | NCI-H292 none | 21.3 | 16.4 |
| gamma | | | | | |
| LAK cells IL-2 + IL-18 | 23.2 | 12.2 | NCI-H292 IL-4 | 16.2 | 15.3 |
| LAK cells PMA/ | 2.1 | 11.9 | NCI-H292 IL-9 | 21.3 | 9.5 |
| ionomycin | | | | | |
| NK Cells IL-2 rest | 31.0 | 100.0 | NCI-H292 IL-13 | 14.8 | 69.7 |
| Two Way MLR 3 day | 21.9 | 42.3 | NCI-H292 IFN gamma | 9.6 | 36.9 |
| Two Way MLR 5 day | 14.2 | 12.6 | HPAEC none | 0.3 | 2.0 |
| Two Way MLR 7 day | 14.8 | 9.6 | HPAEC TNF alpha + IL-1 beta | 0.0 | 0.5 |
| PBMC rest | 10.2 | 9.1 | Lung fibroblast none | 0.0 | 0.0 |
| PBMC PWM | 34.6 | 19.8 | Lung fibroblast TNF alpha + IL-1 | 0.0 | 0.0 |
| | | | beta | | |
| PBMC PHA-L | 45.1 | 32.1 | Lung fibroblast IL-4 | 0.5 | 0.0 |
| Ramos (B cell) none | 18.0 | 18.8 | Lung fibroblast IL-9 | 0.0 | 0.0 |
| Ramos (B cell) | 37.9 | 11.0 | Lung fibroblast IL-13 | 0.3 | 0.0 |
| ionomycin | | | | | |
| B lymphocytes PWM | 51.8 | 14.0 | Lung fibroblast IFN gamma | 0.1 | 0.6 |
| B lymphocytes CD40L | 32.3 | 20.2 | Dermal fibroblast CCD1070 rest | 0.4 | 0.0 |
| and IL-4 | | | | | |
| EOL-1 dbcAMP | 7.3 | 18.6 | Dermal fibroblast CCD1070 TNF | 30.8 | 13.8 |
| | | | alpha | | |
| EOL-1 dbcAMP | 0.2 | 2.5 | Dermal fibroblast CCD1070 IL-1 | 0.0 | 0.0 |
| PMA/ionomycin | | | beta | | |
| Dendritic cells none | 2.0 | 4.8 | Dermal fibroblast IFN gamma | 0.0 | 0.0 |
| Dendritic cells LPS | 0.4 | 0.0 | Dermal fibroblast IL-4 | 0.2 | 0.0 |
| Dendritic cells anti-CD40 | 0.3 | 0.6 | IBD Colitis 2 | 4.0 | 5.5 |
| Monocytes rest | 0.8 | 0.0 | IBD Crohn's | 2.0 | 4.5 |
| Monocytes LPS | 2.1 | 1.5 | Colon | 22.5 | 20.2 |
| Macrophages rest | 10.4 | 5.2 | Lung | 5.8 | 5.7 |
| Macrophages LPS | 0.5 | 1.8 | Thymus | 2.5 | 2.0 |
| HUVEC none | 0.0 | 0.0 | Kidney | 7.7 | 7.0 |
| HUVEC starved | 0.0 | 0.0 | | | |

Panel 1.3D Summary: Gpcr41 Expression of the CG101340-01 gene was highest in hippocampus (CT=29.1) and occured at moderate to low levels throughout the brain. This gene encodes a putative GPCR. Several neurotransmitter receptors are GPCRs, including the dopamine receptor family, the serotonin receptor family, the GABAB receptor, muscarinic acetylcholine receptors, and others; thus this GPCR represents novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therapeutic modulation of this gene or its protein product is beneficial in the treatment of one or more of these diseases, as is stimulation and/or blockade of the receptor coded for by the gene.

This gene was also moderately expressed in a number of other normal tissues including colon, ovary and spleen. Expression of this gene was higher in normal cells than in cancer cell lines and CG101340-01 expression was downregulated in ovarian and brain cancer cell lines. Expression of this gene or its protein product is useful as a marker to distinguish normal tissue from ovarian or brain tumors.

Among tissues with metabolic or endocrine function, this gene was expressed at low levels in adipose, adrenal gland, thyroid, pituitary gland, skeletal muscle, heart, and liver. Therapeutic modulation of the activity of this gene or its protein product is useful in the treatment of endocrine/metabolically related diseases, such as obesity and diabetes.

In addition, expression of the CG101340-01 gene was upregulated in fetal skeletal muscle and heart when compared to adult tissues. The overexpression of this gene in fetal tissue shows that the protein product enhances skeletal muscle or heart growth and development in the fetus and also acts in a regenerative capacity in the adult. Therapeutic modulation of this gene or its protein product is useful in the treatment of muscular dystrophies or heart disease.

Panel 4D Summary: Gpcr41 Expression of the CG101340-01 gene was upregulated in several tissues and cell types after activation, including lymphocytes, keratinocytes, basophils, small airway epithelium and T cells. The GPCR encoded by this gene functions in the inflammatory process by promoting leukocyte extravasation or initiating a signaling cascade that results in the release of immunomodulatory products such as cytokines. Antibody or small molecule therapeutics designed against the protein encoded by this gene are useful for the reduction or inhibition of inflammation due to psoriasis, delayed type hypersensitivity, asthma, or emphysema.

B. CG101396-01: Glutamate Receptor Delta-1

Expression of gene CG101396-01 was assessed using the primer-probe set Ag4211, described in Table BA. Results of the RTQ-PCR runs are shown in Tables BB, BC and BD.

TABLE BA
Probe Name Ag4211

| | | | Start | SEQ ID |
|---|---|---|---|---|
| Primers | Sequences | Length | Position | No |
| Forward | 5′-aggacagttcgaatccctatgt-3′ | 22 | 1239 | 1136 |
| Probe | TET-5′-ccagtttgaaatccttggcactacct-3′- | 26 | 1261 | 1137 |
| | TAMRA | | | |
| Reverse | 5′-gcatgtctttgccaaaagtct3′ | 21 | 1293 | 1138 |

TABLE BB
General_screening_panel_v1.4
Column A - Rel. Exp.(%) Ag4211, Run 221252466

| Tissue Name | A | Tissue Name | A |
|---|---|---|---|
| Adipose | 4.6 | Renal ca. TK-10 | 0.0 |
| Melanoma* Hs688(A).T | 2.2 | Bladder | 6.2 |
| Melanoma* Hs688(B).T | 1.8 | Gastric ca. (liver met.) NCI-N87 | 0.0 |
| Melanoma* M14 | 0.0 | Gastric ca. KATO III | 0.1 |
| Melanoma* LOXIMVI | 0.0 | Colon ca. SW-948 | 0.0 |
| Melanoma* SK-MEL-5 | 18.6 | Colon ca. SW480 | 14.2 |
| Squamous cell carcinoma | 0.2 | Colon ca.* (SW480 met) SW620 | 0.0 |
| SCC-4 | | | |
| Testis Pool | 5.2 | Colon ca. HT29 | 0.0 |
| Prostate ca.* (bone met) | 0.2 | Colon ca. HCT-116 | 19.2 |
| PC-3 | | | |
| Prostate Pool | 3.2 | Colon ca. CaCo-2 | 58.6 |
| Placenta | 6.0 | Colon cancer tissue | 2.6 |
| Uterus Pool | 6.0 | Colon ca. SW1116 | 0.0 |
| Ovarian ca. OVCAR-3 | 3.3 | Colon ca. Colo-205 | 0.0 |
| Ovarian ca. SK-OV-3 | 97.3 | Colon ca. SW-48 | 0.0 |
| Ovarian ca. OVCAR-4 | 0.1 | Colon Pool | 4.6 |
| Ovarian ca. OVCAR-5 | 10.6 | Small Intestine Pool | 6.9 |
| Ovarian ca. IGROV-1 | 16.6 | Stomach Pool | 7.2 |
| Ovarian ca. OVCAR-8 | 3.2 | Bone Marrow Pool | 7.9 |
| Ovary | 2.9 | Fetal Heart | 15.6 |
| Breast ca. MCF-7 | 0.2 | Heart Pool | 5.3 |
| Breast ca. MDA-MB-231 | 0.0 | Lymph Node Pool | 20.6 |
| Breast ca. BT 549 | 1.0 | Fetal Skeletal Muscle | 5.8 |
| Breast ca. T47D | 15.0 | Skeletal Muscle Pool | 2.5 |
| Breast ca. MDA-N | 0.1 | Spleen Pool | 0.0 |
| Breast Pool | 3.8 | Thymus Pool | 6.8 |
| Trachea | 3.3 | CNS cancer (glio/astro) | 0.1 |
| | | U87-MG | |
| Lung | 1.2 | CNS cancer (glio/astro) | 0.3 |
| | | U-118-MG | |
| Fetal Lung | 52.5 | CNS cancer (neuro; met) | 2.5 |
| | | SK-N-AS | |
| Lung ca. NCI-N417 | 1.0 | CNS cancer (astro) SF-539 | 0.0 |
| Lung ca. LX-1 | 0.2 | CNS cancer (astro) SNB-75 | 9.7 |
| Lung ca. NCI-H146 | 14.3 | CNS cancer (glio) SNB-19 | 17.6 |
| Lung ca. SHP-77 | 0.0 | CNS cancer (glio) SF-295 | 3.1 |
| Lung ca. A549 | 2.0 | Brain (Amygdala) Pool | 63.3 |
| Lung ca. NCI-H526 | 0.5 | Brain (cerebellum) | 60.7 |
| Lung ca. NCI-H23 | 21.8 | Brain (fetal) | 100.0 |
| Lung ca. NCI-H460 | 1.0 | Brain (Hippocampus) Pool | 52.1 |
| Lung ca. HOP-62 | 0.0 | Cerebral Cortex Pool | 83.5 |
| Lung ca. NCI-H522 | 6.0 | Brain (Substantia nigra) Pool | 74.7 |
| Liver | 0.1 | Brain (Thalamus) Pool | 90.8 |
| Fetal Liver | 0.6 | Brain (whole) | 77.9 |
| Liver ca. HepG2 | 0.0 | Spinal Cord Pool | 64.6 |
| Kidney Pool | 21.5 | Adrenal Gland | 13.7 |
| Fetal Kidney | 4.3 | Pituitary gland Pool | 1.5 |
| Renal ca. 786-0 | 0.0 | Salivary Gland | 1.0 |
| Renal ca. A498 | 14.7 | Thyroid (female) | 26.1 |
| Renal ca. ACHN | 0.1 | Pancreatic ca. CAPAN2 | 26.1 |
| Renal ca. UO-31 | 0.0 | Pancreas Pool | 9.9 |

TABLE BC
Panel 4.1D
Column A - Rel. Exp.(%) Ag4211, Run 174261196

| Tissue Name | A | Tissue Name | A |
|---|---|---|---|
| Secondary Th1 act | 0.0 | HUVEC IL-1 beta | 0.0 |
| Secondary Th2 act | 1.1 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 2.5 |
| Secondary Th2 rest | 0.0 | HUVEC IL-11 | 0.0 |
| Secondary Tr1 rest | 0.0 | Lung Microvascular EC none | 58.6 |
| Primary Th1 act | 2.5 | Lung Microvascular EC TNFalpha + IL- | 41.2 |
| | | 1 beta | |
| Primary Th2 act | 0.0 | Microvascular Dermal EC none | 8.1 |
| Primary Tr1 act | 0.0 | Microsvasular Dermal EC TNFalpha + IL- | 0.0 |
| | | 1 beta | |
| Primary Th1 rest | 0.0 | Bronchial epithelium TNFalpha + IL1 beta | 0.0 |
| Primary Th2 rest | 0.0 | Small airway epithelium none | 1.7 |
| Primary Tr1 rest | 0.0 | Small airway epithelium TNFalpha + IL- | 0.0 |
| | | 1 beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL-1 beta | 0.0 |
| CD8 lymphocyte act | 0.0 | Astrocytes rest | 9.2 |
| Secondary CD8 lymphocyte rest | 0.0 | Astrocytes TNFalpha + IL-1 beta | 6.6 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 1.3 |
| CH11 | | | |
| LAK cells rest | 0.0 | CCD1106 (Keratinocytes) TNFalpha + IL- | 0.0 |
| | | 1 beta | |
| LAK cells IL-2 | 0.0 | Liver cirrhosis | 3.2 |
| LAK cells IL-2 + IL-12 | 1.4 | NCI-H292 none | 0.0 |
| LAK cells IL-2 + IFN gamma | 1.4 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 2.7 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 beta | 2.0 |
| Two Way MLR 7 day | 0.0 | Lung fibroblast none | 4.2 |
| PBMC rest | 0.0 | Lung fibroblast TNF alpha + IL-1 beta | 0.0 |
| PBMC PWM | 0.0 | Lung fibroblast IL-4 | 1.6 |
| PBMC PHA-L | 0.0 | Lung fibroblast IL-9 | 4.6 |
| Ramos (B cell) none | 29.1 | Lung fibroblast IL-13 | 2.5 |
| Ramos (B cell) ionomycin | 14.4 | Lung fibroblast IFN gamma | 1.4 |
| B lymphocytes PWM | 0.0 | Dermal fibroblast CCD1070 rest | 5.3 |
| B lymphocytes CD40L and IL-4 | 0.0 | Dermal fibroblast CCD1070 TNF alpha | 2.7 |
| EOL-1 dbcAMP | 5.0 | Dermal fibroblast CCD1070 IL-1 beta | 0.0 |
| EOL-1 dbcAMP | 4.2 | Dermal fibroblast IFN gamma | 1.3 |
| PMA/ionomycin | | | |
| Dendritic cells none | 0.0 | Dermal fibroblast IL-4 | 0.0 |
| Dendritic cells LPS | 0.0 | Dermal Fibroblasts rest | 4.4 |
| Dendritic cells anti-CD40 | 1.0 | Neutrophils TNFa + LPS | 0.0 |
| Monocytes rest | 0.0 | Neutrophils rest | 2.6 |
| Monocytes LPS | 0.0 | Colon | 9.5 |
| Macrophages rest | 0.8 | Lung | 77.4 |
| Macrophages LPS | 0.0 | Thymus | 18.7 |
| HUVEC none | 0.0 | Kidney | 100.0 |
| HUVEC starved | 0.0 | | |

TABLE BD
general oncology screening panel_v_2.4
Column A - Rel. Exp.(%) Ag4211, Run 268624930

| Tissue Name | A | Tissue Name | A |
|---|---|---|---|
| Colon cancer 1 | 21.0 | Bladder NAT 2 | 0.0 |
| Colon NAT 1 | 7.5 | Bladder NAT 3 | 0.0 |
| Colon cancer 2 | 4.2 | Bladder NAT 4 | 4.9 |
| Colon NAT 2 | 6.9 | Prostate adenocarcinoma 1 | 40.1 |
| Colon cancer 3 | 8.2 | Prostate adenocarcinoma 2 | 6.1 |
| Colon NAT 3 | 17.0 | Prostate adenocarcinoma 3 | 13.7 |
| Colon malignant cancer 4 | 20.9 | Prostate adenocarcinoma 4 | 5.7 |
| Colon NAT 4 | 6.1 | Prostate NAT 5 | 0.7 |
| Lung cancer 1 | 5.3 | Prostate adenocarcinoma 6 | 9.2 |
| Lung NAT 1 | 4.1 | Prostate adenocarcinoma 7 | 5.6 |
| Lung cancer 2 | 20.7 | Prostate adenocarcinoma 8 | 0.8 |
| Lung NAT 2 | 6.9 | Prostate adenocarcinoma 9 | 18.3 |
| Squamous cell carcinoma 3 | 9.9 | Prostate NAT 10 | 1.6 |
| Lung NAT 3 | 0.0 | Kidney cancer 1 | 27.2 |
| Metastatic melanoma 1 | 46.7 | Kidney NAT 1 | 20.9 |
| Melanoma 2 | 2.4 | Kidney cancer 2 | 100.0 |
| Melanoma 3 | 8.1 | Kidney NAT 2 | 9.8 |
| Metastatic melanoma 4 | 37.9 | Kidney cancer 3 | 12.1 |
| Metastatic melanoma 5 | 94.0 | Kidney NAT 3 | 1.4 |
| Bladder cancer 1 | 0.7 | Kidney cancer 4 | 21.6 |
| Bladder NAT 1 | 0.0 | Kidney NAT 4 | 7.5 |
| Bladder cancer 2 | 0.0 | | |

General_screening_panel_v1.4 Summary: Ag4211 Highest expression of the CG101396-01 gene was seen in the fetal brain (CT=29). This gene was expressed at moderate levels in all regions of the CNS examined. This gene encodes a protein that is homologous to the delta2 glutamate receptor, which is expressed in the cerebellum. This receptor is involved in motor learning and coordination, and synapse plasticity. Based on the prominent expression of this gene product in the CNS, therapeutic modulation of the expression or function of this gene product is useful for the treatment of CNS disorders involving memory deficits, including Alzheimer's disease and aging as well as for motor impairments and learning following stroke-related brain damage.

Among tissues with metabolic function, this gene was expressed at moderate to low levels in pituitary, adipose, adrenal gland, pancreas, thyroid, and adult and fetal skeletal muscle and heart. This widespread expression among these tissues shows that this gene product plays a role in normal neuroendocrine and metabolic function and that disregulated expression of this gene contributes to neuroendocrine disorders or metabolic diseases, such as obesity and diabetes.

In addition, moderate levels of expression were seen in a cluster of samples derived from ovarian, colon, melanoma and lung cancer cell lines. Thus, expression of this gene is useful as a marker to detect the presence of these cancers. Therapeutic modulation of the expression or function of this gene or gene product is effective in the treatment of ovarian, colon, melanoma and lung cancers.

This gene was also expressed at much higher levels in fetal lung tissue (CT=30) when compared to expression in the adult counterpart (CT=35.5). Expression of this gene is useful as a marker to differentiate between the fetal and adult source of this tissue.

Panel 4.1D Summary: Ag4211 Highest expression of this gene was seen in the kidney (CT=30.8). Moderate levels of expression were also seen in the lung and untreated lung microvascular endothelial cells. Low but significant levels of expression were seen in untreated and treated astrocytes and Ramos B cells and activated lung microvascular endothelial cells. Expression in astrocytes was in agreement with the prominent CNS expression seen in Panel 1.4. This expression demonstrates that this gene product is involved in the homeostasis of the lung and kidney. Therapeutic modulation of the expression of this protein is useful for restoring or maintaining function in these organs during inflammation.

general oncology screening panel_v_{—}2.4 Summary: Ag4211 Expression of the CG101396-01 gene was detected in a kidney cancer sample (CT=31). Moderate to low expression of this gene was detected in melanoma and prostate cancers. Expression of this gene or its protein product is useful as a marker to detect the presence of these cancers. Therapeutic modulation of the expression or function of this gene product is effective in the treatment of melanoma, kidney and lung cancers.

C. CG102348-01: C1r-Like Proteinase Precursor

Expression of gene CG102348-01 was assessed using the primer-probe set Ag650, described in Table CA. Results of the RTQ-PCR runs are shown in Table CB.

TABLE GA
Probe Name Ag650

| | | | Start | SEQ ID |
|---|---|---|---|---|
| Primers | Sequences | Length | Position | No |
| Forward | 5′-gttttcattgcattgcatttct-3′ | 22 | 1883 | 1139 |
| Probe | TET-5′-cattcctaagaccctttagttgaccttca- | 29 | 1909 | 1140 |
| | 3′-TAMRA | | | |
| Reverse | 5′-atcttggagctgcagaatagct-3′ | 22 | 1946 | 1141 |

TABLE CB
Panel 1.1
Column A - Rel. Exp.(%) Ag650, Run 109485832

| Tissue Name | A | Tissue Name | A |
|---|---|---|---|
| Adrenal gland | 39.2 | Renal ca. UO-31 | 8.8 |
| Bladder | 42.6 | Renal ca. RXF 393 | 2.2 |
| Brain (amygdala) | 0.7 | Liver | 76.8 |
| Brain (cerebellum) | 2.1 | Liver (fetal) | 17.4 |
| Brain (hippocampus) | 2.8 | Liver ca. (hepatoblast) HepG2 | 4.0 |
| Brain (substantia nigra) | 6.0 | Lung | 9.0 |
| Brain (thalamus) | 2.3 | Lung (fetal) | 19.9 |
| Cerebral Cortex | 4.0 | Lung ca. (non-s. cell) HOP-62 | 100.0 |
| Brain (fetal) | 2.9 | Lung ca. (large cell) | 11.3 |
| | | NCI-H460 | |
| Brain (whole) | 2.4 | Lung ca. (non-s. cell) | 23.0 |
| | | NCI-H23 | |
| glio/astro U-118-MG | 5.4 | Lung ca. (non-s. cl) | 56.3 |
| | | NCI-H522 | |
| astrocytoma SF-539 | 11.6 | Lung ca. (non-sm. cell) A549 | 22.5 |
| astrocytoma SNB-75 | 2.9 | Lung ca. (s. cell var.) SHP-77 | 0.5 |
| astrocytoma SW1783 | 1.5 | Lung ca. (small cell) LX-1 | 14.3 |
| glioma U251 | 6.9 | Lung ca. (small cell) | 10.3 |
| | | NCI-H69 | |
| glioma SF-295 | 19.5 | Lung ca. (squam.) SW 900 | 10.0 |
| glioma SNB-19 | 5.4 | Lung ca. (squam.) NCI-H596 | 5.5 |
| glio/astro U87-MG | 6.3 | Lymph node | 11.1 |
| neuro*; met SK-N-AS | 11.9 | Spleen | 6.6 |
| Mammary gland | 18.0 | Thymus | 2.8 |
| Breast ca. BT-549 | 1.4 | Ovary | 13.6 |
| Breast ca. MDA-N | 6.6 | Ovarian ca. IGROV-1 | 5.5 |
| Breast ca.* (pl. ef) T47D | 3.5 | Ovarian ca. OVCAR-3 | 12.7 |
| Breast ca.* (pl. ef) MCF-7 | 0.0 | Ovarian ca. OVCAR-4 | 3.2 |
| Breast ca.* (pl. ef) | 0.9 | Ovarian ca. OVCAR-5 | 19.3 |
| MDA-MB-231 | | | |
| Small intestine | 17.2 | Ovarian ca. OVCAR-8 | 8.0 |
| Colorectal | 3.3 | Ovarian ca. (ascites) | 6.3 |
| | | SK-OV-3 | |
| Colon ca. HT29 | 6.6 | Pancreas | 72.7 |
| Colon ca. CaCo-2 | 14.8 | Pancreatic ca. CAPAN 2 | 2.8 |
| Colon ca. HCT-15 | 4.7 | Pituitary gland | 14.7 |
| Colon ca. HCT-116 | 2.6 | Placenta | 5.7 |
| Colon ca. HCC-2998 | 11.7 | Prostate | 12.1 |
| Colon ca. SW480 | 4.3 | Prostate ca.* (bone met) PC-3 | 5.7 |
| Colon ca.* SW620 | 6.4 | Salivary gland | 49.3 |
| (SW480 met) | | | |
| Stomach | 10.4 | Trachea | 12.1 |
| Gastric ca. (liver met) | 27.7 | Spinal cord | 5.1 |
| NCI-N87 | | | |
| Heart | 17.4 | Testis | 2.2 |
| Skeletal muscle (Fetal) | 5.2 | Thyroid | 11.5 |
| Skeletal muscle | 11.3 | Uterus | 15.6 |
| Endothelial cells | 12.1 | Melanoma M14 | 2.1 |
| Heart (Fetal) | 1.2 | Melanoma LOX IMVI | 0.3 |
| Kidney | 20.9 | Melanoma UACC-62 | 4.4 |
| Kidney (fetal) | 11.6 | Melanoma SK-MEL-28 | 9.3 |
| Renal ca. 786-0 | 10.8 | Melanoma* (met) SK-MEL-5 | 1.6 |
| Renal ca. A498 | 9.0 | Melanoma Hs688(A).T | 1.6 |
| Renal ca. ACHN | 8.1 | Melanoma* (met) Hs688(B).T | 4.6 |
| Renal ca. TK-10 | 11.5 | | |

Panel 1.1 Summary: Ag650 Highest expression of this gene was detected in a lung cancer HOP-62 cell line (CT=22). High levels of expression of this gene were also seen in cluster of cancer cell lines derived from pancreatic, gastric, colon, lung, liver, renal, breast, ovarian, prostate, melanoma and brain cancers. Epression of this gene is useful as a marker to detect the presence of these cancers. Therapeutic modulation of the expression or function of this gene is effective in the treatment of pancreatic, gastric, colon, lung, liver, renal, breast, ovarian, prostate, squamous cell carcinoma, melanoma and brain cancers.

Among tissues with metabolic or endocrine function, this gene was expressed at high levels in pancreas, adrenal gland, thyroid, pituitary gland, skeletal muscle, heart, liver and the gastrointestinal tract. Therapeutic modulation of the activity of this gene or its protein product is useful in the treatment of endocrine/metabolically related diseases, such as obesity and diabetes.

In addition, this gene was expressed at high levels in all regions of the central nervous system examined, including amygdala, hippocampus, substantia nigra, thalamus, cerebellum, cerebral cortex, and spinal cord. Therapeutic modulation of this gene product is useful in the treatment of central nervous system disorders such as Alzheimer's disease, Parkinson's disease, epilepsy, multiple sclerosis, schizophrenia and depression.

D. CG125860-02: Transmembrane Protease, Serine 5

Expression of gene CG125860-02 was assessed using the primer-probe set Ag1674, described in Table DA. Results of the RTQ-PCR runs are shown in Tables DC, DD, DE and DF.

TABLE DA
Probe Name Ag1674

| | | | Start | SEQ ID |
|---|---|---|---|---|
| Primers | Sequences | Length | Position | No |
| Forward | 5′-ctcactcaccacaagggagtaa-3′ | 22 | 612 | 1142 |
| Probe | TET-5′-tgacatcaaactcaacagttcccagga- | 27 | 641 | 1143 |
| | 3′-TAMRA | | | |
| Reverse | 5′-gtctaggagagagctgagcaaa-3′ | 22 | 669 | 1144 |

TABLE DB
Ardais Prostate 1.0
Column A - Rel. Exp.(%) Ag1674, Run 370302285

| Tissue Name | A |
|---|---|
| 151135 Prostate NAT(B87) | 31.2 |
| 151143 Prostate NAT(B8A) | 27.7 |
| 153669 Prostate NAT(D5E) | 26.4 |
| 153677 Prostate NAT(D66) | 12.8 |
| 153685 Prostate NAT(D6E) | 14.7 |
| 145905 Prostate NAT(A0C) | 4.0 |
| 153670 Prostate NAT(D5F) | 18.0 |
| 153678 Prostate NAT(D67) | 24.0 |
| 153686 Prostate NAT(D6F) | 6.1 |
| 145906 Prostate NAT(A09) | 28.7 |
| 151129 Prostate NAT(B93) | 44.8 |
| 151137 Prostate NAT(B86) | 42.3 |
| 153671 Prostate NAT(D60) | 18.0 |
| 151145 Prostate NAT(B91) | 30.8 |
| 153679 Prostate NAT(D68) | 12.9 |
| 153687 Prostate NAT(D70) | 31.4 |
| 153672 Prostate NAT(D61) | 23.8 |
| 153680 Prostate NAT(D69) | 35.1 |
| 151131 Prostate NAT(B85) | 7.4 |
| 153673 Prostate NAT(D62) | 12.0 |
| 153681 Prostate NAT(D6A) | 11.5 |
| 145910 Prostate NAT(9C3) | 27.4 |
| 153674 Prostate NAT(D63) | 28.7 |
| 153682 Prostate NAT(D6B) | 14.4 |
| 151133 Prostate NAT(B94) | 31.9 |
| 153675 Prostate NAT(D64) | 20.6 |
| 153683 Prostate NAT(D6C) | 31.9 |
| 153668 Prostate NAT(D5D) | 39.5 |
| 153676 Prostate NAT(D65) | 18.7 |
| 153684 Prostate NAT(D6D) | 30.4 |
| 145904 Prostate cancer(9E2) | 40.3 |
| 149776 Prostate cancer(AD5) | 100.0 |
| 153653 Prostate cancer(D4E) | 26.1 |
| 153661 Prostate cancer(D56) | 48.3 |
| 151128 Prostate cancer(B8C) | 47.3 |
| 151136 Prostate cancer(B8B) | 86.5 |
| 151144 Prostate cancer(B8F) | 25.3 |
| 153654 Prostate cancer(D4F) | 45.7 |
| 153662 Prostate cancer(D57) | 16.0 |
| 153655 Prostate cancer(D50) | 44.8 |
| 145907 Prostate cancer(A0A) | 18.3 |
| 153663 Prostate cancer(D58) | 20.7 |
| 151130 Prostate cancer(B90) | 54.7 |
| 153648 Prostate cancer(D49) | 24.7 |
| 153656 Prostate cancer(D51) | 11.1 |
| 153664 Prostate cancer(D59) | 26.2 |
| 155799 Prostate cancer(EA8) | 14.8 |
| 145909 Prostate cancer(9E7) | 18.6 |
| 153649 Prostate cancer(D4A) | 16.8 |
| 153657 Prostate cancer(D52) | 28.7 |
| 153665 Prostate cancer(D5A) | 25.7 |
| 151132 Prostate cancer(B88) | 60.7 |
| 153650 Prostate cancer(D4B) | 14.9 |
| 153658 Prostate cancer(D53) | 23.8 |
| 153666 Prostate cancer(D5B) | 22.5 |
| 153651 Prostate cancer(D4C) | 22.4 |
| 153659 Prostate cancer(D54) | 41.8 |
| 153667 Prostate cancer(D5C) | 43.5 |
| 151134 Prostate cancer(B92) | 51.1 |
| 151142 Prostate cancer(B89) | 33.7 |
| 153652 Prostate cancer(D4D) | 36.3 |
| 153660 Prostate cancer(D55) | 18.3 |
| 149773 Prostate NAT(AD8) | 0.0 |
| 149774 Prostate cancer(AD7) | 34.9 |
| 151139 Prostate NAT(B8E) | 35.8 |
| 151138 Prostate cancer(B8D) | 30.4 |
| 115141 Prostate NAT(B96) | 24.5 |
| 151140 Prostate cancer(B95) | 24.0 |

TABLE DC
Panel 1.3D
Column A - Rel. Exp.(%) Ag1674, Run 158189117

| Tissue Name | A |
|---|---|
| Liver adenocarcinoma | 2.7 |
| Pancreas | 0.7 |
| Pancreatic ca. CAPAN 2 | 0.4 |
| Adrenal gland | 1.3 |
| Thyroid | 0.2 |
| Salivary gland | 5.4 |
| Pituitary gland | 3.4 |
| Brain (fetal) | 3.4 |
| Brain (whole) | 7.1 |
| Brain (amygdala) | 28.1 |
| Brain (cerebellum) | 3.6 |
| Brain (hippocampus) | 100.0 |
| Brain (substantia nigra) | 6.9 |
| Brain (thalamus) | 20.0 |
| Cerebral Cortex | 9.7 |
| Spinal cord | 21.9 |
| glio/astro U87-MG | 0.6 |
| glio/astro U-118-MG | 4.1 |
| astrocytoma SW1783 | 0.8 |
| neuro*; met SK-N-AS | 3.6 |
| astrocytoma SF-539 | 1.6 |
| astrocytoma SNB-75 | 3.1 |
| glioma SNB-19 | 19.6 |
| glioma U251 | 1.9 |
| glioma SF-295 | 1.9 |
| Heart (Fetal) | 0.7 |
| Heart | 0.4 |
| Skeletal muscle (Fetal) | 40.3 |
| Skeletal muscle | 0.8 |
| Bone marrow | 0.7 |
| Thymus | 1.4 |
| Spleen | 2.4 |
| Lymph node | 0.9 |
| Colorectal | 1.0 |
| Stomach | 1.8 |
| Small intestine | 1.6 |
| Colon ca. SW480 | 0.6 |
| Colon ca.* SW620 (SW480 met) | 11.8 |
| Colon ca. HT29 | 0.4 |
| Colon ca. HCT-116 | 1.7 |
| Colon ca. CaCo-2 | 1.3 |
| CC Well to Mod Diff (ODO3866) | 1.6 |
| Colon ca. HCC-2998 | 2.1 |
| Gastric ca. (liver met) NCI-N87 | 1.7 |
| Bladder | 0.5 |
| Trachea | 7.4 |
| Kidney | 0.4 |
| Kidney (fetal) | 1.6 |
| Renal ca. 786-0 | 0.4 |
| Renal ca. A498 | 2.6 |
| Renal ca. RXF 393 | 0.3 |
| Renal ca. ACHN | 0.0 |
| Renal ca. UO-31 | 0.3 |
| Renal ca. TK-10 | 0.2 |
| Liver | 0.1 |
| Liver (fetal) | 0.8 |
| Liver ca. (hepatoblast) HepG2 | 1.1 |
| Lung | 2.9 |
| Lung (fetal) | 3.1 |
| Lung ca. (small cell) LX-1 | 5.2 |
| Lung ca. (small cell) NCI-H69 | 4.5 |
| Lung ca. (s. cell var.) SHP-77 | 0.7 |
| Lung ca. (large cell)NCI-H460 | 0.3 |
| Lung ca. (non-sm. cell) A549 | 0.9 |
| Lung ca. (non-s. cell) NCI-H23 | 0.8 |
| Lung ca. (non-s. cell) HOP-62 | 0.7 |
| Lung ca. (non-s. cl) NCI-H522 | 0.2 |
| Lung ca. (squam.) SW 900 | 0.4 |
| Lung ca. (squam.) NCI-H596 | 0.0 |
| Mammary gland | 2.9 |
| Breast ca.* (pl. ef) MCF-7 | 0.9 |
| Breast ca.* (pl. ef) MDA-MB-231 | 2.8 |
| Breast ca.* (pl. ef) T47D | 0.9 |
| Breast ca. BT-549 | 4.5 |
| Breast ca. MDA-N | 9.7 |
| Ovary | 0.5 |
| Ovarian ca. OVCAR-3 | 0.3 |
| Ovarian ca. OVCAR-4 | 1.0 |
| Ovarian ca. OVCAR-5 | 0.9 |
| Ovarian ca. OVCAR-8 | 3.4 |
| Ovarian ca. IGROV-l | 0.7 |
| Ovarian ca. (ascites) SK-OV-3 | 3.2 |
| Uterus | 1.3 |
| Placenta | 0.2 |
| Prostate | 0.9 |
| Prostate ca.* (bone met) PC-3 | 0.5 |
| Testis | 6.2 |
| Melanoma Hs688(A).T | 0.0 |
| Melanoma* (met) Hs688(B).T | 0.0 |
| Melanoma UACC-62 | 2.8 |
| Melanoma M14 | 0.7 |
| Melanoma LOX IMVI | 0.4 |
| Melanoma* (met) SK-MEL-5 | 0.4 |
| Adipose | 0.1 |

TABLE DD
Panel 2D
Column A - Rel. Exp.(%) Ag1674, Run 158191134

| Tissue Name | A | Tissue Name | A |
|---|---|---|---|
| Normal Colon | 7.8 | Kidney Margin 8120608 | 0.3 |
| CC Well to Mod Diff (ODO3866) | 1.9 | Kidney Cancer 8120613 | 5.4 |
| CC Margin (ODO3866) | 1.1 | Kidney Margin 8120614 | 1.6 |
| CC Gr.2 rectosigmoid (ODO3868) | 2.9 | Kidney Cancer 9010320 | 3.1 |
| CC Margin (ODO3868) | 0.4 | Kidney Margin 9010321 | 4.2 |
| CC Mod Diff (ODO3920) | 46.7 | Normal Uterus | 1.4 |
| CC Margin (ODO3920) | 2.7 | Uterine Cancer 064011 | 13.3 |
| CC Gr.2 ascend colon (ODO3921) | 24.8 | Normal Thyroid | 1.9 |
| CC Margin (ODO3921) | 1.2 | Thyroid Cancer | 1.0 |
| CC from Partial Hepatectomy | 10.0 | Thyroid Cancer A302152 | 1.3 |
| (ODO4309) Mets | | | |
| Liver Margin (ODO4309) | 2.4 | Thyroid Margin A302153 | 4.8 |
| Colon mets to lung (OD04451-01) | 50.3 | Normal Breast | 10.4 |
| Lung Margin (OD04451-02) | 2.1 | Breast Cancer | 8.4 |
| Normal Prostate 6546-1 | 1.0 | Breast Cancer (OD04590-01) | 6.0 |
| Prostate Cancer (OD04410) | 2.3 | Breast Cancer Mets (0D04590-03) | 6.2 |
| Prostate Margin (OD04410) | 3.3 | Breast Cancer Metastasis | 20.9 |
| Prostate Cancer (OD04720-01) | 11.4 | Breast Cancer | 4.7 |
| Prostate Margin (OD04720-02) | 6.5 | Breast Cancer | 6.2 |
| Normal Lung | 6.0 | Breast Cancer 9100266 | 3.7 |
| Lung Met to Muscle (ODO4286) | 0.7 | Breast Margin 9100265 | 3.3 |
| Muscle Margin (ODO4286) | 6.4 | Breast Cancer A209073 | 5.7 |
| Lung Malignant Cancer (OD03126) | 3.1 | Breast Margin A209073 | 6.7 |
| Lung Margin (OD03126) | 3.3 | Normal Liver | 1.3 |
| Lung Cancer (OD04404) | 1.5 | Liver Cancer | 3.1 |
| Lung Margin (OD04404) | 3.2 | Liver Cancer 1025 | 1.5 |
| Lung Cancer (OD04565) | 1.1 | Liver Cancer 1026 | 0.8 |
| Lung Margin (OD04565) | 2.3 | Liver Cancer 6004-T | 0.4 |
| Lung Cancer (OD04237-01) | 100.0 | Liver Tissue 6004-N | 2.3 |
| Lung Margin (OD04237-02) | 2.9 | Liver Cancer 6005-T | 0.0 |
| Ocular Mel Met to Liver (ODO4310) | 7.9 | Liver Tissue 6005-N | 0.3 |
| Liver Margin (OD04310) | 0.4 | Normal Bladder | 5.4 |
| Melanoma Metastasis | 47.3 | Bladder Cancer | 5.0 |
| Lung Margin (OD04321) | 3.5 | Bladder Cancer | 2.0 |
| Normal Kidney | 10.2 | Bladder Cancer (OD04718-01) | 1.7 |
| Kidney Ca, Nuclear grade 2 (OD04338) | 4.7 | Bladder Normal Adjacent | 2.4 |
| | | (OD04718-03) | |
| Kidney Margin (OD04338) | 4.9 | Normal Ovary | 0.4 |
| Kidney Ca Nuclear grade 1/2 (OD04339) | 3.5 | Ovarian Cancer | 5.6 |
| Kidney Margin (OD04339) | 1.6 | Ovarian Cancer (OD04768-07) | 2.8 |
| Kidney Ca, Clear cell type (OD04340) | 4.2 | Ovary Margin (OD04768-08) | 2.1 |
| Kidney Margin (OD04340) | 5.0 | Normal Stomach | 3.2 |
| Kidney Ca, Nuclear grade 3 (OD04348) | 2.4 | Gastric Cancer 9060358 | 2.6 |
| Kidney Margin (OD04348) | 3.2 | Stomach Margin 9060359 | 2.4 |
| Kidney Cancer (OD04622-01) | 1.4 | Gastric Cancer 9060395 | 0.5 |
| Kidney Margin (OD04622-03) | 0.7 | Stomach Margin 9060394 | 1.3 |
| Kidney Cancer (OD04450-01) | 0.2 | Gastric Cancer 9060397 | 3.0 |
| Kidney Margin (OD04450-03) | 2.0 | Stomach Margin 9060396 | 0.0 |
| Kidney Cancer 8120607 | 0.4 | Gastric Cancer 064005 | 6.6 |

TABLE DE
Panel 4D
Column A - Rel. Exp.(%) Ag1674, Run 158191436

| Tissue Name | A | Tissue Name | A |
|---|---|---|---|
| Secondary Th1 act | 18.4 | HUVEC IL-1 beta | 4.2 |
| Secondary Th2 act | 22.7 | HUVEC IFN gamma | 34.2 |
| Secondary Tr1 act | 33.2 | HUVEC TNF alpha + IFN gamma | 22.1 |
| Secondary Th1 rest | 10.5 | HUVEC TNF alpha + IL4 | 14.9 |
| Secondary Th2 rest | 6.9 | HUVEC IL-11 | 6.8 |
| Secondary Tr1 rest | 24.8 | Lung Microvascular EC none | 26.2 |
| Primary Th1 act | 28.1 | Lung Microvascular EC TNFalpha + IL- | 22.7 |
| | | 1 beta | |
| Primary Th2 act | 21.6 | Microvascular Dermal EC none | 33.7 |
| Primary Tr1 act | 29.5 | Microsvasular Dermal EC TNFalpha + IL- | 21.3 |
| | | 1 beta | |
| Primary Th1 rest | 77.4 | Bronchial epithelium TNFalpha + IL1 beta | 31.4 |
| Primary Th2 rest | 31.9 | Small airway epithelium none | 5.9 |
| Primary Tr1 rest | 22.2 | Small airway epithelium TNFalpha + IL- | 40.3 |
| | | 1 beta | |
| CD45RA CD4 lymphocyte act | 22.4 | Coronery artery SMC rest | 10.5 |
| CD45RO CD4 lymphocyte act | 57.8 | Coronery artery SMC TNFalpha + IL-1 beta | 5.4 |
| CD8 lymphocyte act | 20.2 | Astrocytes rest | 22.1 |
| Secondary CD8 lymphocyte rest | 31.2 | Astrocytes TNFalpha + IL-1 beta | 6.9 |
| Secondary CD8 lymphocyte act | 10.7 | KU-812 (Basophil) rest | 48.3 |
| CD4 lymphocyte none | 17.0 | KU-812 (Basophil) PMA/ionomycin | 79.6 |
| 2ry Th1/Th2/Tr1 anti-CD95 | 40.6 | CCD1106 (Keratinocytes) none | 14.7 |
| CH11 | | | |
| LAK cells rest | 26.1 | CCD1106 (Keratinocytes) TNFalpha + IL- | 16.8 |
| | | 1 beta | |
| LAK cells IL-2 | 22.2 | Liver cirrhosis | 20.6 |
| LAK cells IL-2 + IL-12 | 17.1 | Lupus kidney | 4.1 |
| LAK cells IL-2 + IFN gamma | 16.0 | NCI-H292 none | 75.3 |
| LAK cells IL-2 + IL-18 | 23.5 | NCI-H292 IL-4 | 70.2 |
| LAK cells PMA/ionomycin | 7.2 | NCI-H292 IL-9 | 27.9 |
| NK Cells IL-2 rest | 19.9 | NCI-H292 IL-13 | 40.3 |
| Two Way MLR 3 day | 35.1 | NCI-H292 IFN gamma | 33.7 |
| Two Way MLR 5 day | 5.8 | HPAEC none | 12.1 |
| Two Way MLR 7 day | 11.0 | HPAEC TNF alpha + IL-1 beta | 19.9 |
| PBMC rest | 10.7 | Lung fibroblast none | 9.3 |
| PBMC PWM | 57.4 | Lung fibroblast TNF alpha + IL-1 beta | 15.8 |
| PBMC PHA-L | 18.3 | Lung fibroblast IL-4 | 20.3 |
| Ramos (B cell) none | 14.4 | Lung fibroblast IL-9 | 14.8 |
| Ramos (B cell) ionomycin | 11.9 | Lung fibroblast IL-13 | 18.3 |
| B lymphocytes PWM | 35.4 | Lung fibroblast IFN gamma | 16.0 |
| B lymphocytes CD40L and IL-4 | 29.3 | Dermal fibroblast CCD1070 rest | 21.0 |
| EOL-1 dbcAMP | 39.8 | Dermal fibroblast CCD1070 TNF alpha | 38.7 |
| EOL-1 dbcAMP | 39.0 | Dermal fibroblast CCD1070 IL-1 beta | 9.3 |
| PMA/ionomycin | | | |
| Dendritic cells none | 6.3 | Dermal fibroblast IFN gamma | 17.4 |
| Dendritic cells LPS | 7.4 | Dermal fibroblast IL-4 | 35.4 |
| Dendritic cells anti-CD40 | 6.8 | IBD Colitis 2 | 9.2 |
| Monocytes rest | 13.9 | IBD Crohn's | 1.2 |
| Monocytes LPS | 8.4 | Colon | 35.4 |
| Macrophages rest | 37.6 | Lung | 18.9 |
| Macrophages LPS | 2.3 | Thymus | 43.5 |
| HUVEC none | 15.2 | Kidney | 100.0 |
| HUVEC starved | 35.8 | | |

Ardais Prostate 1.0 Summary: Ag1674 Expression of the CG125860-02 gene was highest in a prostate cancer sample (CT=29.3). This gene was expressed at moderate levels in the majority of samples on this panel, with no apparent disregulation in prostate cancer.

Panel 1.3D Summary: Ag1674 Expression of this gene was highest in the hippocampus (CT=29.3). In addition, this gene was expressed at moderate levels in all other regions of the central nervous system examined, including amygdala, substantia nigra, thalamus, cerebellum, cerebral cortex, and spinal cord. Expression of this gene or its protein product is useful as a marker for brain tissue. Therapeutic modulation of the activity of this gene or its protein product plays a role in central nervous system disorders such as Alzheimer's disease, Parkinson's disease, epilepsy, multiple sclerosis, schizophrenia and depression.

Expression of the CG125860-02 gene was also upregulated in fetal skeletal muscle compared to adult skeletal muscle. The relative overexpression of this gene in fetal tissue demonstrated that the protein product enhances skeletal muscle growth or development in the fetus and also acts in a regenerative capacity in the adult. Therapeutic modulation of this gene or its protein product is useful in the treatment of muscle degenerative diseases, such as muscular dystrophy.

Panel 2D Summary: Ag1674 Expression of this gene was highest in a lung cancer sample (CT=29.1) and was significantly downregulated (about 50-fold) in the normal adjacent lung tissue. The CG125860-02 gene was also overexpressed in 4 colon cancer samples when compared to the appropriate normal matched colon tissue. Therefore, the CG125860-02 gene or protein expression levels are useful as a marker for lung or colon cancer. Gene, protein, antibody or small molecule therapeutics targeting this gene or its protein product are useful in the treatment of lung or colon cancer. Expression of this gene was higher in a number of metastatic tumor samples on this panel, showing that it plays a role in metastasis and is useful as a marker of disease prognosis.

Panel 4D Summary: Ag1674 Expression of this gene was highest in normal kidney (CT=31.8). This gene was expressed at low but ubiquitous levels in the majority of the samples on this panel.

E. CG50235-04: Tolloid-Like 2

Expression of gene CG50235-04 was assessed using the primer-probe set Ag4737, described in Table EA. Results of the RTQ-PCR runs are shown in Tables EB and EC.

TABLE EB
General_screening_panel_v1.4
Column A - Rel. Exp.(%) Ag4737, Run 222904895

| Tissue Name | A | Tissue Name | A |
|---|---|---|---|
| Adipose | 0.0 | Renal ca. TK-10 | 1.1 |
| Melanoma* Hs688(A).T | 4.3 | Bladder | 6.8 |
| Melanoma* Hs688(B).T | 1.3 | Gastric ca. (liver met.) NCI-N87 | 5.2 |
| Melanoma* M14 | 0.0 | Gastric ca. KATO III | 2.0 |
| Melanoma* LOXIMVI | 0.0 | Colon ca. SW-948 | 0.0 |
| Melanoma* SK-MEL-5 | 1.0 | Colon ca. SW480 | 9.9 |
| Squamous cell carcinoma | 7.3 | Colon ca.* (SW480 met) SW620 | 0.3 |
| SCC-4 | | | |
| Testis Pool | 0.9 | Colon ca. HT29 | 0.0 |
| Prostate ca.* (bone met) | 0.5 | Colon ca. HCT-116 | 8.6 |
| PC-3 | | | |
| Prostate Pool | 5.6 | Colon ca. CaCo-2 | 0.5 |
| Placenta | 2.0 | Colon cancer tissue | 0.5 |
| Uterus Pool | 0.8 | Colon ca. SW1116 | 16.2 |
| Ovarian ca. OVCAR-3 | 9.1 | Colon ca. Colo-205 | 1.1 |
| Ovarian ca. SK-OV-3 | 67.8 | Colon ca. SW-48 | 0.0 |
| Ovarian ca. OVCAR-4 | 88.3 | Colon Pool | 0.0 |
| Ovarian ca. OVCAR-5 | 0.0 | Small Intestine Pool | 6.9 |
| Ovarian ca. IGROV-1 | 6.6 | Stomach Pool | 1.2 |
| Ovarian ca. OVCAR-8 | 8.7 | Bone Marrow Pool | 0.0 |
| Ovary | 3.3 | Fetal Heart | 26.1 |
| Breast ca. MCF-7 | 0.8 | Heart Pool | 21.9 |
| Breast ca. MDA-MB-231 | 1.1 | Lymph Node Pool | 1.6 |
| Breast ca. BT 549 | 0.0 | Fetal Skeletal Muscle | 2.7 |
| Breast ca. T47D | 3.1 | Skeletal Muscle Pool | 17.3 |
| Breast ca. MDA-N | 0.0 | Spleen Pool | 2.4 |
| Breast Pool | 0.3 | Thymus Pool | 1.4 |
| Trachea | 5.5 | CNS cancer (glio/astro) | 0.0 |
| | | U87-MG | |
| Lung | 0.9 | CNS cancer (glio/astro) | 0.0 |
| | | U-118-MG | |
| Fetal Lung | 0.5 | CNS cancer (neuro; met) | 2.9 |
| | | SK-N-AS | |
| Lung ca. NCI-N417 | 0.0 | CNS cancer (astro) SF-539 | 0.5 |
| Lung ca. LX-1 | 0.5 | CNS cancer (astro) SNB-75 | 1.4 |
| Lung ca. NCI-H146 | 0.9 | CNS cancer (glio) SNB-19 | 5.3 |
| Lung ca. SHP-77 | 44.4 | CNS cancer (glio) SF-295 | 0.0 |
| Lung ca. A549 | 0.9 | Brain (Amygdala) Pool | 7.8 |
| Lung ca. NCI-H526 | 54.7 | Brain (cerebellum) | 3.6 |
| Lung ca. NCI-H23 | 8.8 | Brain (fetal) | 1.1 |
| Lung ca. NCI-H460 | 0.3 | Brain (Hippocampus) Pool | 7.2 |
| Lung ca. HOP-62 | 0.0 | Cerebral Cortex Pool | 5.6 |
| Lung ca. NCI-H522 | 8.0 | Brain (Substantia nigra) Pool | 19.2 |
| Liver | 0.5 | Brain (Thalamus) Pool | 18.0 |
| Fetal Liver | 2.0 | Brain (whole) | 6.3 |
| Liver ca. HepG2 | 1.0 | Spinal Cord Pool | 42.0 |
| Kidney Pool | 1.4 | Adrenal Gland | 0.0 |
| Fetal Kidney | 1.2 | Pituitary gland Pool | 7.8 |
| Renal ca. 786-0 | 100.0 | Salivary Gland | 7.4 |
| Renal ca. A498 | 17.0 | Thyroid (female) | 1.8 |
| Renal ca. ACHN | 45.1 | Pancreatic ca. CAPAN2 | 0.0 |
| Renal ca. UO-31 | 82.9 | Pancreas Pool | 2.8 |

TABLE EC
Panel 4.1D
Column A - Rel. Exp.(%) Ag4737, Run 204154022

| Tissue Name | A | Tissue Name | A |
|---|---|---|---|
| Secondary Th1 act | 0.0 | HUVEC IL-1 beta | 0.0 |
| Secondary Th2 act | 0.0 | HUVEC IFN gamma | 3.3 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1 beta | |
| Primary Th2 act | 2.5 | Microvascular Dermal EC none | 0.0 |
| Primary Tr1 act | 0.0 | Microsvasular Dermal EC TNFalpha + IL- | 0.0 |
| | | 1 beta | |
| Primary Th1 rest | 0.0 | Bronchial epithelium TNFalpha + IL1 beta | 39.8 |
| Primary Th2 rest | 0.0 | Small airway epithelium none | 43.2 |
| Primary Tr1 rest | 0.0 | Small airway epithelium TNFalpha + IL- | 12.3 |
| | | 1 beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL-1 beta | 0.0 |
| CD8 lymphocyte act | 0.0 | Astrocytes rest | 73.7 |
| Secondary CD8 lymphocyte rest | 0.0 | Astrocytes TNFalpha + IL-1 beta | 100.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 2.7 |
| 2ry Th1/Th2/Tr1 anti-CD95 | 0.0 | CCD1106 (Keratinocytes) none | 29.5 |
| CH11 | | | |
| LAK cells rest | 0.0 | CCD1106 (Keratinocytes) TNFalpha + IL- | 5.6 |
| | | 1 beta | |
| LAK cells IL-2 | 0.0 | Liver cirrhosis | 0.0 |
| LAK cells IL-2 + IL-12 | 0.0 | NCI-H292 none | 12.5 |
| LAK cells IL-2 + IFN gamma | 0.0 | NCI-H292 IL-4 | 0.0 |
| LAK cells IL-2 + IL-18 | 0.0 | NCI-H292 IL-9 | 11.3 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 beta | 2.9 |
| Two Way MLR 7 day | 0.0 | Lung fibroblast none | 0.0 |
| PBMC rest | 0.0 | Lung fibroblast TNF alpha + IL-1 beta | 0.0 |
| PBMC PWM | 3.6 | Lung fibroblast IL-4 | 0.0 |
| PBMC PHA-L | 0.0 | Lung fibroblast IL-9 | 4.9 |
| Ramos (B cell) none | 3.0 | Lung fibroblast IL-13 | 0.0 |
| Ramos (B cell) ionomycin | 2.4 | Lung fibroblast IFN gamma | 3.4 |
| B lymphocytes PWM | 0.0 | Dermal fibroblast CCD1070 rest | 0.0 |
| B lymphocytes CD40L and IL-4 | 3.1 | Dermal fibroblast CCD1070 TNF alpha | 0.0 |
| EOL-1 dbcAMP | 0.0 | Dermal fibroblast CCD1070 IL-1 beta | 0.0 |
| EOL-1 dbcAMP | 0.0 | Dermal fibroblast IFN gamma | 0.0 |
| PMA/ionomycin | | | |
| Dendritic cells none | 2.7 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 4.1 |
| Macrophages rest | 0.8 | Lung | 0.0 |
| Macrophages LPS | 0.0 | Thymus | 2.5 |
| HUVEC none | 0.0 | Kidney | 2.7 |
| HUVEC starved | 0.0 | | |

General_screening_panel_v1.4 Summary: Ag4737 Highest expression of this gene was seen in a sample derived from a renal cancer cell line (CT=29.9). This gene showed specific expression restricted to cell lines derived from renal cancer, ovarian cancer and lung cancer. The expression of this gene is useful as marker to detect these cancers. Modulation of this gene, encoded protein and use of small molecule drugs or antibodies is useful in the treatment of renal, ovarian or lung cancer.

This gene was also moderately expressed in several metabolic tissues including adult and fetal heart, pituitary, and skeletal muscle. Thus, this gene is important for the pathogenesis, diagnosis and/or treatment of metabolic diseases, including obesity.

This gene was expressed at low levels in the CNS, except in the spinal cord where expression levels were moderate. Thus, modulation of this gene is useful in treating spinal cord related disorders including spinal cord trauma or spinocerebellar ataxia.

Panel 4.1D Summary: Ag4737 This gene was expressed at moderate levels in TNF-alpha and IL-1 beta treated and resting astrocytes (CTs=32). It was also expressed at a low level in small airway epithelium, and keratinocytes. Expression of this gene was down regulated in both cell types upon treatment with the inflammatory cytokines TNF-alpha and IL-1 beta. This gene encodes for a tolloid like 2 protein, a BMP-1-related proteinase which was shown to play a role in extracellular matrix biosynthesis (Uzel M I, J Biol Chem 276(25):22537-43). Therefore, modulation of this gene, encoded protein and/or use of antibodies or small molecule drug targeting this gene or gene product is useful to reduce or eliminate the symptoms of inflammatory reactions that occur in multiple sclerosis, and also in chronic obstructive pulmonary disease, asthma, or emphysema, and in inflammatory skin diseases.

F. CG50249-01: Voltage-Gated Potassium Channel Protein KV3.2 (KSHIIIA)

Expression of gene CG50249-01 was assessed using the primer-probe set Ag2503, described in Table FA. Results of the RTQ-PCR runs are shown in Tables FB, FC, FD, FE, FF and FG.

TABLE FA
Probe Name Ag2503

| | | | Start | SEQ ID |
|---|---|---|---|---|
| Primers | Sequences | Length | Position | No |
| Forward | 5′-gaggctctctccagtaacatca-3′ | 22 | 1851 | 1148 |
| Probe | TET-5′-actctccttgtcctctgaggcgctct-3′- | 26 | 1880 | 1149 |
| | TAMRA | | | |
| Reverse | 5′-gcagtttggttgtttggtttac-3′ | 22 | 1929 | 1150 |

TABLE FB
Ardais Breast 1.0
Column A - Rel. Exp.(%) Ag2503, Run 399643618

| Tissue Name | A | Tissue Name | A |
|---|---|---|---|
| 111297 Breast cancer metastasis (9369)* | 0.0 | 153636 Breast cancer (D3D) | 0.0 |
| 108830 Breast cancer metastasis (OD06855)* | 0.0 | 164668 Breast cancer (6314) | 0.0 |
| 97764 Breast cancer node metastasis (OD06083) | 0.0 | 164677 Breast cancer (5272) | 0.3 |
| 97739 Breast cancer (CHTN20676) | 0.0 | 164685 Breast cancer (0170) | 0.0 |
| 145848 Breast cancer (9B6) | 0.2 | 98857 Breast cancer (OD06397-12) | 0.0 |
| 145859 Breast cancer (9EC) | 0.0 | 153628 Breast cancer (D35) | 0.0 |
| 153632 Breast cancer (D39) | 0.0 | 153637 Breast cancer (D3E) | 0.5 |
| 153643 Breast cancer (D44) | 0.1 | 164669 Breast cancer (6992) | 0.0 |
| 164672 Breast cancer (7464) | 0.0 | 164678 Breast cancer (5297) | 0.2 |
| 164681 Breast cancer (5787) | 97.3 | 164686 Breast cancer (0732) | 0.0 |
| 97748 Breast cancer (CHTN20931) | 0.8 | 145857 Breast cancer (9F0) | 0.0 |
| 145850 Breast cancer (9C7) | 0.0 | 153630 Breast cancer (D37) | 0.1 |
| 149844 Breast cancer (24178) | 0.0 | 153638 Breast cancer (D3F) | 27.4 |
| 153633 Breast cancer (D3A) | 0.1 | 164670 Breast cancer (7078) | 0.0 |
| 153644 Breast cancer (D45) | 0.3 | 164679 Breast cancer (5486) | 0.0 |
| 164673 Breast cancer (8452) | 0.6 | 164687 Breast cancer (5881) | 0.0 |
| 164682 Breast cancer (6342)* | 0.1 | 145846 Breast cancer (9B7) | 0.1 |
| 97751 Breast cancer (CHTN21053) | 0.0 | 145858 Breast cancer (9B4) | 0.0 |
| 116417 Breast cancer (3367)* | 0.0 | 153631 Breast cancer (D38) | 0.0 |
| 145852 Breast cancer (A1A) | 0.0 | 153639 Breast cancer (D40) | 100.0 |
| 151097 Breast cancer (CHTN24298) | 0.0 | 164671 Breast cancer (7082) | 0.0 |
| 153634 Breast cancer (D3B) | 0.0 | 164680 Breast cancer (5705) | 0.0 |
| 155797 Breast cancer (EA6) | 0.0 | 164688 Breast cancer (7222) | 0.0 |
| 164674 Breast cancer (8811) | 0.2 | 111288 Breast NAT (3367) | 0.0 |
| 164683 Breast cancer (6470) | 0.0 | 111302 Breast NAT (6314) | 0.4 |
| 97763 Breast cancer (OD06083) | 0.1 | 105687 Breast cancer 1B | 0.0 |
| 116418 Breast cancer (3378)* | 0.0 | 105688 Breast NAT 1A | 0.0 |
| 145853 Breast cancer (9F3) | 0.0 | 105689 Breast cancer 2B | 0.0 |
| 153432 Breast cancer (CHTN 24652) | 0.2 | 105690 Breast NAT 2A | 0.0 |
| 153635 Breast cancer (D3C) | 0.0 | 111289 Breast cancer 3B* | 0.0 |
| 164667 Breast cancer (5785) | 0.0 | 111290 Breast NAT 3A* | 0.0 |
| 164676 Breast cancer (5070) | 1.4 | 116424 Breast cancer 4B* | 0.0 |
| 164684 Breast cancer (6509) | 0.2 | 116425 Breast NAT 4A | 0.0 |
| 116421 Breast cancer (6314) | 0.0 | 108847 Breast cancer | 0.0 |
| 145854 Breast cancer (9B8) | 0.0 | 105694 Breast NAT | 0.0 |
| 153627 Breast cancer (D34) | 0.0 | | |

TABLE FC
Ardais Panel v.1.0
Column A - Rel. Exp.(%) Ag2503, Run 263526557

| Tissue Name | A | Tissue Name | A |
|---|---|---|---|
| 136799 Lung cancer(362) | 0.0 | 136787 lung cancer(356) | 0.0 |
| 136800 LungNAT(363) | 1.1 | 136788 lung NAT(357) | 0.2 |
| 136813 Lung cancer(372) | 0.1 | 136804 Lung cancer(369) | 100.0 |
| 136814 Lung NAT(373) | 0.0 | 136805 Lung NAT(36A) | 0.0 |
| 136815 Lung cancer(374) | 0.1 | 136806 Lung cancer(36B) | 0.0 |
| 136816 Lung NAT(375) | 0.1 | 136807 Lung NAT(36C) | 0.0 |
| 136791 Lung cancer(35A) | 0.2 | 136789 lung cancer(358) | 0.2 |
| 136795 Lung cancer(35E) | 0.1 | 136802 Lung cancer(365) | 0.1 |
| 136797 Lung cancer(360) | 0.1 | 136803 Lung cancer(368) | 0.0 |
| 136794 lung NAT(35D) | 0.2 | 136811 Lung cancer(370) | 0.0 |
| 136818 Lung NAT(377) | 10.0 | 136810 Lung NAT(36F) | 0.2 |

TABLE FD
Ardais Prostate 1.0
Column A - Rel. Exp.(%) Ag2503, Run 320416115

| Tissue Name | A | Tissue Name | A |
|---|---|---|---|
| 151135 Prostate NAT(B87) | 0.8 | 151128 Prostate cancer(B8C) | 0.3 |
| 151143 Prostate NAT(B8A) | 2.5 | 151136 Prostate cancer(B8B) | 100.0 |
| 153669 Prostate NAT(D5E) | 2.3 | 151144 Prostate cancer(B8F) | 0.1 |
| 153677 Prostate NAT(D66) | 2.5 | 153654 Prostate cancer(D4F) | 10.2 |
| 153685 Prostate NAT(D6E) | 1.7 | 153662 Prostate cancer(D57) | 2.9 |
| 145905 Prostate NAT(A0C) | 8.6 | 153655 Prostate cancer(D50) | 8.8 |
| 153670 Prostate NAT(D5F) | 1.2 | 145907 Prostate cancer(A0A) | 1.0 |
| 153678 Prostate NAT(D67) | 4.2 | 153663 Prostate cancer(D58) | 1.0 |
| 153686 Prostate NAT(D6F) | 1.2 | 151130 Prostate cancer(B90) | 27.7 |
| 145906 Prostate NAT(A09) | 6.0 | 153648 Prostate cancer(D49) | 0.2 |
| 151129 Prostate NAT(B93) | 4.1 | 153656 Prostate cancer(D51) | 8.8 |
| 151137 Prostate NAT(B86) | 2.1 | 153664 Prostate cancer(D59) | 0.1 |
| 153671 Prostate NAT(D60) | 0.7 | 155799 Prostate cancer(EA8) | 0.1 |
| 151145 Prostate NAT(B91) | 1.4 | 145909 Prostate cancer(9E7) | 0.7 |
| 153679 Prostate NAT(D68) | 5.9 | 153649 Prostate cancer(D4A) | 11.0 |
| 153687 Prostate NAT(D70) | 4.1 | 153657 Prostate cancer(D52) | 36.3 |
| 153672 Prostate NAT(D61) | 1.0 | 153665 Prostate cancer(D5A) | 0.3 |
| 153680 Prostate NAT(D69) | 6.1 | 151132 Prostate cancer(B88) | 41.5 |
| 151131 Prostate NAT(B85) | 3.5 | 153650 Prostate cancer(D4B) | 3.1 |
| 153673 Prostate NAT(D62) | 6.9 | 153658 Prostate cancer(D53) | 12.3 |
| 153681 Prostate NAT(D6A) | 0.8 | 153666 Prostate cancer(D5B) | 26.8 |
| 145910 Prostate NAT(9C3) | 7.5 | 153651 Prostate cancer(D4C) | 0.5 |
| 153674 Prostate NAT(D63) | 5.3 | 153659 Prostate cancer(D54) | 25.0 |
| 153682 Prostate NAT(D6B) | 0.1 | 153667 Prostate cancer(D5C) | 3.5 |
| 151133 Prostate NAT(B94) | 4.0 | 151134 Prostate cancer(B92) | 4.7 |
| 153675 Prostate NAT(D64) | 0.2 | 151142 Prostate cancer(B89) | 0.2 |
| 153683 Prostate NAT(D6C) | 0.5 | 153652 Prostate cancer(D4D) | 1.7 |
| 153668 Prostate NAT(D5D) | 1.5 | 153660 Prostate cancer(D55) | 0.1 |
| 153676 Prostate NAT(D65) | 0.9 | 149773 Prostate NAT(AD8) | 2.3 |
| 153684 Prostate NAT(D6D) | 9.3 | 149774 Prostate cancer(AD7) | 2.8 |
| 145904 Prostate cancer(9E2) | 0.0 | 151139 Prostate NAT(B8E) | 4.3 |
| 149776 Prostate cancer(AD5) | 2.0 | 151138 Prostate cancer(B8D) | 0.1 |
| 153653 Prostate cancer(D4E) | 1.0 | 115141 Prostate NAT(B96) | 4.2 |
| 153661 Prostate cancer(D56) | 1.7 | 151140 Prostate cancer(B95) | 1.2 |

TABLE FE
General_screening_panel_v1.4
Column A - Rel. Exp.(%) Ag2503, Run 208015585
Column B - Rel. Exp.(%) Ag2503, Run 212142287

| Tissue Name | A | B | Tissue Name | A | B |
|---|---|---|---|---|---|
| Adipose | 0.0 | 0.0 | Renal ca. TK-10 | 0.0 | 0.0 |
| Melanoma* Hs688(A).T | 0.0 | 0.0 | Bladder | 0.1 | 0.1 |
| Melanoma* Hs688(B).T | 0.0 | 0.0 | Gastric ca. (liver met.) NCI-N87 | 0.1 | 0.1 |
| Melanoma* M14 | 0.0 | 0.0 | Gastric ca. KATO III | 0.0 | 0.0 |
| Melanoma* LOXIMVI | 0.0 | 0.1 | Colon ca. SW-948 | 0.0 | 0.0 |
| Melanoma* SK-MEL-5 | 0.0 | 0.1 | Colon ca. SW480 | 0.0 | 0.1 |
| Squamous cell carcinoma SCC-4 | 0.0 | 0.0 | Colon ca.* (SW480 met) SW620 | 0.0 | 0.0 |
| Testis Pool | 0.2 | 0.3 | Colon ca. HT29 | 0.1 | 0.1 |
| Prostate ca.* (bone met) PC-3 | 0.0 | 0.0 | Colon ca. HCT-116 | 0.0 | 0.0 |
| Prostate Pool | 6.4 | 7.8 | Colon ca. CaCo-2 | 0.0 | 0.0 |
| Placenta | 0.0 | 0.0 | Colon cancer tissue | 0.1 | 0.2 |
| Uterus Pool | 0.0 | 0.0 | Colon ca. SW1116 | 0.0 | 0.0 |
| Ovarian ca. OVCAR-3 | 0.0 | 0.0 | Colon ca. Colo-205 | 0.0 | 0.0 |
| Ovarian ca. SK-OV-3 | 0.0 | 0.1 | Colon ca. SW-48 | 0.0 | 0.0 |
| Ovarian ca. OVCAR-4 | 0.0 | 0.0 | Colon Pool | 0.2 | 0.1 |
| Ovarian ca. OVCAR-5 | 8.4 | 7.2 | Small Intestine Pool | 0.2 | 0.4 |
| Ovarian ca. IGROV-1 | 0.0 | 0.0 | Stomach Pool | 0.2 | 0.0 |
| Ovarian ca. OVCAR-8 | 0.0 | 0.0 | Bone Marrow Pool | 0.0 | 0.0 |
| Ovary | 0.0 | 0.1 | Fetal Heart | 0.0 | 0.0 |
| Breast ca. MCF-7 | 0.0 | 0.2 | Heart Pool | 0.0 | 0.1 |
| Breast ca. MDA-MB-231 | 0.0 | 0.0 | Lymph Node Pool | 0.1 | 0.1 |
| Breast ca. BT 549 | 0.0 | 0.0 | Fetal Skeletal Muscle | 0.1 | 0.0 |
| Breast ca. T47D | 8.1 | 15.4 | Skeletal Muscle Pool | 0.0 | 0.1 |
| Breast ca. MDA-N | 0.0 | 0.0 | Spleen Pool | 0.0 | 0.0 |
| Breast Pool | 0.9 | 0.5 | Thymus Pool | 0.4 | 0.7 |
| Trachea | 0.2 | 0.4 | CNS cancer (glio/astro) U87-MG | 0.0 | 0.0 |
| Lung | 0.0 | 0.0 | CNS cancer (glio/astro) U-118-MG | 0.1 | 0.1 |
| Fetal Lung | 0.0 | 0.1 | CNS cancer (neuro; met) SK-N-AS | 0.0 | 0.0 |
| Lung ca. NCI-N417 | 0.0 | 0.0 | CNS cancer (astro) SF-539 | 0.0 | 0.0 |
| Lung ca. LX-1 | 0.0 | 0.0 | CNS cancer (astro) SNB-75 | 0.0 | 0.0 |
| Lung ca. NCI-H146 | 1.8 | 1.8 | CNS cancer (glio) SNB-19 | 0.0 | 0.0 |
| Lung ca. SHP-77 | 0.5 | 0.5 | CNS cancer (glio) SF-295 | 0.0 | 0.0 |
| Lung ca. A549 | 0.0 | 0.0 | Brain (Amygdala) Pool | 55.9 | 49.7 |
| Lung ca. NCI-H526 | 0.0 | 0.0 | Brain (cerebellum) | 1.1 | 1.1 |
| Lung ca. NCI-H23 | 0.0 | 0.9 | Brain (fetal) | 25.9 | 38.4 |
| Lung ca. NCI-H460 | 2.0 | 0.1 | Brain (Hippocampus) Pool | 31.0 | 35.8 |
| Lung ca. HOP-62 | 0.1 | 0.0 | Cerebral Cortex Pool | 100.0 | 80.7 |
| Lung ca. NCI-H522 | 0.0 | 0.0 | Brain (Substantia nigra) Pool | 64.2 | 64.6 |
| Liver | 0.1 | 0.0 | Brain (Thalamus) Pool | 97.3 | 100.0 |
| Fetal Liver | 0.0 | 0.3 | Brain (whole) | 66.9 | 65.5 |
| Liver ca. HepG2 | 0.0 | 0.0 | Spinal Cord Pool | 6.4 | 5.3 |
| Kidney Pool | 0.0 | 0.1 | Adrenal Gland | 0.0 | 0.0 |
| Fetal Kidney | 1.1 | 2.2 | Pituitary gland Pool | 6.6 | 5.5 |
| Renal ca. 786-0 | 0.0 | 0.0 | Salivary Gland | 0.2 | 0.1 |
| Renal ca. A498 | 0.0 | 0.0 | Thyroid (female) | 0.0 | 0.0 |
| Renal ca. ACHN | 0.4 | 0.0 | Pancreatic ca. CAPAN2 | 0.0 | 0.1 |
| Renal ca. UO-31 | 0.0 | 0.0 | Pancreas Pool | 0.2 | 0.7 |

TABLE FF
Panel 2D
Column A - Rel. Exp.(%) Ag2503, Run 160838287
Column B - Rel. Exp.(%) Ag2503, Run 164993346

| Tissue Name | A | B | Tissue Name | A | B |
|---|---|---|---|---|---|
| Normal Colon | 2.7 | 2.3 | Kidney Margin 8120608 | 0.0 | 0.0 |
| CC Well to Mod Diff (ODO3866) | 0.1 | 0.2 | Kidney Cancer 8120613 | 0.0 | 0.0 |
| CC Margin (ODO3866) | 0.1 | 0.3 | Kidney Margin 8120614 | 0.0 | 0.1 |
| CC Gr.2 rectosigmoid (ODO3868) | 0.1 | 0.0 | Kidney Cancer 9010320 | 0.0 | 0.1 |
| CC Margin (ODO3868) | 0.0 | 0.1 | Kidney Margin 9010321 | 0.0 | 0.0 |
| CC Mod Diff (ODO3920) | 0.1 | 0.1 | Normal Uterus | 0.0 | 0.0 |
| CC Margin (ODO3920) | 0.0 | 0.1 | Uterine Cancer 064011 | 0.0 | 0.0 |
| CC Gr.2 ascend colon (ODO3921) | 0.1 | 0.2 | Normal Thyroid | 0.0 | 0.1 |
| CC Margin (ODO3921) | 0.2 | 0.0 | Thyroid Cancer | 0.0 | 0.0 |
| CC from Partial Hepatectomy | 0.2 | 0.1 | Thyroid Cancer A302152 | 0.0 | 0.0 |
| (ODO4309) Mets | | | | | |
| Liver Margin (ODO4309) | 0.1 | 0.1 | Thyroid Margin A302153 | 0.0 | 0.0 |
| Colon mets to lung (ODO4451-01) | 0.0 | 0.0 | Normal Breast | 3.1 | 2.4 |
| Lung Margin (ODO4451-02) | 0.0 | 0.0 | Breast Cancer | 0.0 | 0.0 |
| Normal Prostate 6546-1 | 2.8 | 15.9 | Breast Cancer (ODO4590-01) | 100.0 | 71.7 |
| Prostate Cancer (ODO4410) | 22.4 | 30.1 | Breast Cancer Mets | 39.8 | 33.9 |
| | | | (ODO4590-03) | | |
| Prostate Margin (ODO4410) | 9.7 | 9.9 | Breast Cancer Metastasis | 4.5 | 7.1 |
| Prostate Cancer (ODO4720-01) | 6.0 | 8.5 | Breast Cancer | 0.4 | 0.0 |
| Prostate Margin (ODO4720-02) | 3.9 | 3.7 | Breast Cancer | 1.2 | 0.9 |
| Normal Lung | 0.0 | 0.0 | Breast Cancer 9100266 | 93.3 | 100.0 |
| Lung Met to Muscle (ODO4286) | 0.0 | 0.0 | Breast Margin 9100265 | 28.7 | 33.0 |
| Muscle Margin (ODO4286) | 0.0 | 0.0 | Breast Cancer A209073 | 2.9 | 3.4 |
| Lung Malignant Cancer | 0.0 | 0.1 | Breast Margin A209073 | 5.9 | 7.0 |
| (ODO3126) | | | | | |
| Lung Margin (ODO3126) | 0.0 | 0.0 | Normal Liver | 0.4 | 0.4 |
| Lung Cancer (ODO4404) | 0.0 | 0.0 | Liver Cancer | 0.0 | 0.0 |
| Lung Margin (ODO4404) | 0.0 | 0.0 | Liver Cancer 1025 | 0.0 | 0.1 |
| Lung Cancer (ODO4565) | 0.0 | 0.0 | Liver Cancer 1026 | 0.0 | 0.0 |
| Lung Margin (ODO4565) | 0.1 | 0.0 | Liver Cancer 6004-T | 0.1 | 0.0 |
| Lung Cancer (ODO4237-01) | 0.0 | 0.1 | Liver Tissue 6004-N | 0.7 | 0.4 |
| Lung Margin (ODO4237-02) | 0.1 | 0.0 | Liver Cancer 6005-T | 0.0 | 0.0 |
| Ocular Mel Met to Liver | 0.0 | 0.0 | Liver Tissue 6005-N | 0.2 | 0.0 |
| (ODO4310) | | | | | |
| Liver Margin (ODO4310) | 0.0 | 0.1 | Normal Bladder | 0.0 | 0.2 |
| Melanoma Metastasis | 0.0 | 0.0 | Bladder Cancer | 0.1 | 0.0 |
| Lung Margin (ODO4321) | 0.0 | 0.0 | Bladder Cancer | 0.2 | 0.4 |
| Normal Kidney | 0.0 | 0.0 | Bladder Cancer (ODO4718- | 0.0 | 0.0 |
| | | | 01) | | |
| Kidney Ca, Nuclear grade 2 | 0.1 | 0.3 | Bladder Normal Adjacent | 0.0 | 0.0 |
| (ODO4338) | | | (ODO4718-03) | | |
| Kidney Margin (ODO4338) | 0.1 | 0.0 | Normal Ovary | 0.0 | 0.0 |
| Kidney Ca Nuclear grade 1/2 | 0.1 | 0.3 | Ovarian Cancer | 0.0 | 0.0 |
| (ODO4339) | | | | | |
| Kidney Margin (ODO4339) | 0.0 | 0.0 | Ovarian Cancer (ODO4768- | 0.0 | 0.0 |
| | | | 07) | | |
| Kidney Ca, Clear cell type | 0.0 | 0.1 | Ovary Margin (ODO4768-08) | 0.0 | 0.0 |
| (ODO4340) | | | | | |
| Kidney Margin (ODO4340) | 0.0 | 0.0 | Normal Stomach | 0.0 | 0.1 |
| Kidney Ca, Nuclear grade 3 | 0.0 | 0.0 | Gastric Cancer 9060358 | 0.0 | 0.0 |
| (ODO4348) | | | | | |
| Kidney Margin (ODO4348) | 0.0 | 0.0 | Stomach Margin 9060359 | 0.0 | 0.2 |
| Kidney Cancer (ODO4622-01) | 0.0 | 0.0 | Gastric Cancer 9060395 | 0.0 | 0.0 |
| Kidney Margin (ODO4622-03) | 0.0 | 0.1 | Stomach Margin 9060394 | 0.0 | 0.0 |
| Kidney Cancer (ODO4450-01) | 0.0 | 0.0 | Gastric Cancer 9060397 | 0.0 | 0.3 |
| Kidney Margin (ODO4450-03) | 0.0 | 0.0 | Stomach Margin 9060396 | 0.0 | 0.0 |
| Kidney Cancer 8120607 | 0.0 | 0.0 | Gastric Cancer 064005 | 0.1 | 0.7 |

Table FG
Panel 3D
Column A - Rel. Exp.(%) Ag2503, Run 164629451
Column B - Rel. Exp.(%) Ag2503, Run 182113494

| Tissue Name | A | B | Tissue Name | A | B |
|---|---|---|---|---|---|
| 94905 Daoy | 0.0 | 0.0 | 94954 Ca Ski Cervical | 0.0 | 17.9 |
| Medulloblastoma/Cerebellum | | | epidermoid carcinoma | | |
| | | | (metastasis | | |
| 94906 TE671 | 0.0 | 0.0 | 94955 ES-2 Ovarian clear cell | 0.0 | 0.0 |
| Medulloblastoni/Cerebellum | | | carcinoma | | |
| 94907 D283 Med | 7.7 | 12.2 | 94957 Ramos Stimulated with | 0.0 | 0.0 |
| Medulloblastoma/Cerebellum | | | PMA/ionomycin 6h | | |
| 94908 PFSK-1 Primitive | 10.5 | 57.0 | 94958 Ramos Stimulated with | 0.0 | 0.0 |
| Neuroectodermal/Cerebellum | | | PMA/ionomycin 14h | | |
| 94909 XF-498 CNS | 0.0 | 0.0 | 94962 MEG-01 Chronic | 5.9 | 12.3 |
| | | | myelogenous leukemia | | |
| | | | (megokaryoblast) | | |
| 94910 SNB-78 CNS/glioma | 0.0 | 0.0 | 94963 Raji Burkitt's lymphoma | 0.0 | 0.0 |
| 94911 SF-268 CNS/glioblastoma | 0.0 | 0.0 | 94964 Daudi Burkitt's | 0.0 | 0.0 |
| | | | lymphoma | | |
| 94912 T98G Glioblastoma | 0.0 | 0.0 | 94965 U266 B-cell | 0.0 | 0.0 |
| | | | plasmacytoma/myeloma | | |
| 96776 SK-N-SH Neuroblastoma | 0.0 | 7.9 | 94968 CA46 Burkitt's | 0.0 | 0.0 |
| (metastasis) | | | lymphoma | | |
| 94913 SF-295 CNS/glioblastoma | 0.0 | 0.0 | 94970 RL non-Hodgkin's B- | 0.0 | 0.0 |
| | | | cell lymphoma | | |
| 94914 Cerebellum | 24.5 | 63.3 | 94972 JM1 pre-B-cell | 0.0 | 0.0 |
| | | | lymphoma/leukemia | | |
| 96777 Cerebellum | 8.5 | 12.9 | 94973 Jurkat T cell leukemia | 0.0 | 0.0 |
| 94916 NCI-H292 | 0.0 | 0.0 | 94974 TF-1 Erythroleukemia | 2.0 | 0.0 |
| Mucoepidermoid lung carcinoma | | | | | |
| 94917 DMS-114 Small cell lung | 4.1 | 0.0 | 94975 HUT 78 T-cell | 2.7 | 0.0 |
| cancer | | | lymphoma | | |
| 94918 DMS-79 Small cell lung | 1.9 | 0.0 | 94977 U937 Histiocytic | 0.0 | 0.0 |
| cancer/neuroendocrine | | | lymphoma | | |
| 94919 NCI-H146 Small cell lung | 100.0 | 100.0 | 94980 KU-812 Myelogenous | 0.0 | 0.0 |
| cancer/neuroendocrine | | | leukemia | | |
| 94920 NCI-H526 Small cell lung | 0.0 | 0.0 | 769-P-Clear cell renal | 0.0 | 0.0 |
| cancer/neuroendocrine | | | carcinoma | | |
| 94921 NCI-N417 Small cell lung | 0.0 | 6.3 | 94983 Caki-2 Clear cell renal | 0.0 | 0.0 |
| cancer/neuroendocrine | | | carcinoma | | |
| 94923 NCI-H82 Small cell lung | 0.0 | 1.7 | 94984 SW 839 Clear cell renal | 0.0 | 0.0 |
| cancer/neuroendocrine | | | carcinoma | | |
| 94924 NCI-H157 Squamous cell | 0.0 | 0.0 | 94986 G401 Wilms' tumor | 0.0 | 0.0 |
| lung cancer (metastasis) | | | | | |
| 94925 NCI-H1155 Large cell lung | 30.6 | 23.7 | 94987 Hs766T Pancreatic | 0.0 | 0.0 |
| cancer/neuroendocrine | | | carcinoma (LN metastasis) | | |
| 94926 NCI-H1299 Large cell lung | 0.0 | 0.0 | 94988 CAPAN-1 Pancreatic | 0.0 | 0.0 |
| cancer/neuroendocrine | | | adenocarcinoma (liver | | |
| | | | metastasis) | | |
| 94927 NCI-H727 Lung carcinoid | 0.0 | 0.0 | 94989 SU86.86 Pancreatic | 0.0 | 0.0 |
| | | | carcinoma (liver metastasis) | | |
| 94928 NCI-UMC-11 Lung | 13.3 | 6.4 | 94990 BxPC-3 Pancreatic | 0.0 | 0.0 |
| carcinoid | | | adenocarcinoma | | |
| 94929 LX-1 Small cell lung | 0.0 | 57 | 94991 HPAC Pancreatic | 0.0 | 0.0 |
| cancer | | | adenocarcinoma | | |
| 94930 Colo-205 Colon cancer | 0.0 | 0.0 | 94992 MIA PaCa-2 Pancreatic | 0.0 | 0.0 |
| | | | carcinoma | | |
| 94931 KM12 Colon cancer | 1.2 | 0.0 | 94993 CFPAC-1 Pancreatic | 3.8 | 16.0 |
| | | | ductal adenocarcinoma | | |
| 94932 KM20L2 Colon cancer | 0.0 | 0.0 | 94994 PANG-1 Pancreatic | 0.0 | 0.0 |
| | | | epithelioid ductal carcinoma | | |
| 94933 NCI-H716 Colon cancer | 2.8 | 0.0 | 94996 T24 Bladder carcinma | 1.9 | 0.0 |
| | | | (transitional cell | | |
| 94935 SW-48 Colon | 0.0 | 0.0 | 5637- Bladder carcinoma | 0.0 | 0.0 |
| adenocarcinoma | | | | | |
| 94936 SW1116 Colon | 0.0 | 0.0 | 94998 HT-1197 Bladder | 0.0 | 0.0 |
| adenocarcinoma | | | carcinoma | | |
| 94937 LS 174T Colon | 0.0 | 0.0 | 94999 UM-UC-3 Bladder | 0.0 | 0.0 |
| adenocarcinoma | | | carcinma (transitional cell) | | |
| 94938 SW-948 Colon | 0.0 | 0.0 | 95000 A204 | 0.0 | 0.0 |
| adenocarcinoma | | | Rhabdomyosarcoma | | |
| 94939 SW-480 Colon | 0.0 | 0.0 | 95001 HT-1080 Fibrosarcoma | 0.0 | 0.0 |
| adenocarcinoma | | | | | |
| 94940 NCI-SNU-5 Gastric | 0.0 | 0.0 | 95002 MG-63 Osteosarcoma | 0.0 | 0.0 |
| carcinoma | | | (bone) | | |
| KATO III- Gastric carcinoma | 5.5 | 15.3 | 95003 SK-LMS-1 | 1.3 | 0.0 |
| | | | Leiomyosarcoma (vulva) | | |
| 94943 NCI-SNU-16 Gastric | 0.0 | 0.0 | 95004 SJRH30 | 2.5 | 0.0 |
| carcinoma | | | Rhabdomyosarcoma (met to | | |
| | | | bone marrow) | | |
| 94944 NCI-SNU-1 Gastric | 0.0 | 0.0 | 95005 A431 Epidermoid | 0.0 | 0.0 |
| carcinoma | | | carcinoma | | |
| 94946 RF-1 Gastric | 0.0 | 0.0 | 95007 WM266-4 Melanoma | 1.6 | 0.0 |
| adenocarcinoma | | | | | |
| 94947 RF-48 Gastric | 0.0 | 0.0 | DU 145- Prostate carcinoma | 0.0 | 0.0 |
| adenocarcinoma | | | (brain metastasis) | | |
| 96778 MKN-45 Gastric carcinoma | 0.0 | 0.0 | 95012 MDA-MB-468 Breast | 0.0 | 0.0 |
| | | | adenocarcinoma | | |
| 94949 NCI-N87 Gastric | 0.0 | 0.0 | SCC-4- Squamous cell | 0.0 | 0.0 |
| carcinoma | | | carcinoma of tongue | | |
| 94951 OVCAR-5 Ovarian | 3.1 | 0.0 | SCC-9- Squamous cell | 0.0 | 0.0 |
| carcinoma | | | carcinoma of tongue | | |
| 94952 RL95-2 Uterine carcinoma | 0.0 | 0.0 | SCC-15 Squamous cell | 0.0 | 0.0 |
| | | | carcinoma of tongue | | |
| 94953 HelaS3 Cervical | 0.0 | 0.0 | 95017 CAL 27 Squamous cell | 0.0 | 0.0 |
| adenocarcinoma | | | carcinoma of tongue | | |

Ardais Breast1.0 Summary: Ag2503 Highest expression of this gene was seen in three breast cancer samples (CTs=23.9-25). Modulation of this gene, encoded protein and/or use of antibodies or small molecule drug targeting this gene or gene product is of use in the treatment of breast cancers.

Ardais Panel v.1.0 Summary: Ag2503 Highest expression of this gene was seen in a lung cancer (369) sample (CT=25). Modulation of this gene, encoded protein and/or use of antibodies or small molecule drug targeting this gene or gene product is of use in the treatment of lung cancers.

Ardais Prostate 1.0 Summary: Ag2503 Highest expression of this gene was seen in a prostate cancer (B8B) sample (CT=21). This gene showed relatively higher expression in the prostate cancer samples compared to the other samples on this panel. Modulation of this gene, encoded protein and/or use of antibodies or small molecule drug targeting this gene or gene product is of use in the treatment of prostate cancers.

General_screening_panel_v1.4 Summary: Ag2503 Highest expression of this gene was detected in the thalamus and the cerebral cortex (CTs=25). This gene showed brain preferential expression, with high expression in hippocampus, cortex, amygdala, substantia nigra and thalamus. These regions are susceptible to the neurodegeneration associated with Alzheimer's Disease, Parkinson's disease, Huntington's disease and other pathological neurodegenerative conditions. This gene encodes a protein that is homologous to a potassium channel. Potassium channels play a role in neurodegenerative diseases, including Alzheimer's Disease (Chi X Neurosci Lett 2000 Aug. 18; 290(1):9-12; Yu SP Neurobiol Dis 1998 August; 5(2):81-8). Therefore, modulation of this gene or its protein product is useful to reduce the neuronal degeneration in patients with Alzheimer's Disease and other neurodegenerative diseases. Defective potassium channels are known to cause several CNS disorders, including epilepsy and episodic ataxia with myokymia. Therefore, modulation of this gene and/or expressed protein is useful as a treatment for the symptoms produced by ataxia and epilepsy.

Moderate to low expression was also seen in normal prostate and in cell lines derived from breast, lung, and ovarian cancer. Thus, expression of this gene is useful as a diagnostic marker to detect the presence of these cancers. Use of antibodies or small molecule drug targeting this gene or gene product is useful in the treatment of these cancers.

This gene showed significantly higher levels of expression in the fetal kidney (CTs=30-31) relative to the adult kidney (CTs=35-36). The higher levels of expression in the fetal kidney demonstrate that this gene product is involved in the development of this organ. Modulation of this gene and/or encoded protein is useful in the treatment of kidney related diseases such as lupus erythematus and glomerulonephritis.

Among tissues with metabolic function, the expression of this potassium channel homolog was highest in the pituitary gland. Potassium channels are involved in regulation of secretion in pituitary cells and their modulation by use of small molecule inhibitors or antibodies is important to modulate specific secretory activities in the pituitary.

Panel 2D Summary: Ag2503 The highest level of expression was seen in a breast cancer sample (CTs=25-27). Higher expression of this gene was seen in breast and prostate cancer samples compared to the corresponding normal adjacent tissue. Expression of this gene is useful as a diagnostic marker of these cancers and use of antibodies or small molecule drug targeting this gene or gene product is useful in the treatment of these cancers.

Panel 3D Summary: Ag2503 The highest level of expression was seen in a lung cancer cell line (NCI-H146) (CTs=30-33.4). Low expression of this gene was observed in normal cerebellum and cell lines derived from lung cancer, and medulloblastoma. The expression of this gene is useful as a diagnostic marker for lung cancer and use of antibodies, protein therapeutics or small molecule drug is beneficial in the treatment of lung cancer.

G. CG50307-03: Steroid Dehydrogenase

Expression of full-length physical clone CG50307-03 was assessed using the primer-probe sets Ag2248 and Ag2548, described in Tables GA and GB. Results of the RTQ-PCR runs are shown in Tables GC, GD, GE, GF and GG.

TABLE GA
Probe Name Ag2248

| | | | Start | SEQ ID |
|---|---|---|---|---|
| Primers | Sequences | Length | Position | No |
| Forward | 5′-agcctacgctgaagagttagc-3′ | 21 | 804 | 1151 |
| Probe | TET-5′-aagccgaggtctcaatataatcctga-3′- | 26 | 778 | 1152 |
| | TAMRA | | | |
| Reverse | 5′-acctgcaacttctcctcgtt-3′ | 20 | 750 | 1153 |

TABLE GC
Panel 1.3D
Column A - Rel. Exp.(%) Ag2248, Run 159035206
Column B - Rel. Exp.(%) Ag2548, Run 162292266

| Tissue Name | A | B | Tissue Name | A | B |
|---|---|---|---|---|---|
| Liver adenocarcinoma | 8.7 | 25.5 | Kidney (fetal) | 5.0 | 14.1 |
| Pancreas | 1.1 | 1.7 | Renal Ca. 786-0 | 5.3 | 7.5 |
| Pancreatic ca. CAPAN-2 | 2.2 | 6.3 | Renal Ca. A498 | 10.2 | 9.0 |
| Adrenal gland | 6.6 | 6.0 | Renal Ca. RXF 393 | 1.5 | 9.0 |
| Thyroid | 9.0 | 19.3 | Renal ca. ACHN | 1.7 | 12.8 |
| Salivary gland | 2.9 | 3.7 | Renal ca. UO-31 | 5.4 | 15.1 |
| Pituitary gland | 19.8 | 16.2 | Renal Ca. TK-10 | 1.5 | 6.7 |
| Brain (fetal) | 28.3 | 14.3 | Liver | 1.3 | 0.4 |
| Brain (whole) | 22.7 | 25.2 | Liver (fetal) | 3.1 | 2.3 |
| Brain (amygdala) | 24.7 | 24.3 | Liver ca. (hepatoblast) HepG2 | 8.1 | 18.7 |
| Brain (cerebellum) | 11.6 | 14.6 | Lung | 10.2 | 7.4 |
| Brain (hippocampus) | 100.0 | 45.1 | Lung (fetal) | 9.5 | 12.5 |
| Brain (substantia nigra) | 5.1 | 7.2 | Lung Ca. (small cell) LX-1 | 11.0 | 16.0 |
| Brain (thalamus) | 19.2 | 25.2 | Lung Ca. (small cell) NCI-H69 | 7.5 | 6.3 |
| Cerebral Cortex | 44.8 | 100.0 | Lung Ca. (s.cell var.) SHP-77 | 42.9 | 73.7 |
| Spinal cord | 4.8 | 14.9 | Lung Ca. (large cell) NCI-H460 | 2.7 | 10.1 |
| glio/astro U87-MG | 11.7 | 42.3 | Lung Ca. (non-sm. cell) A549 | 1.8 | 4.1 |
| glio/astro U-118-MG | 20.7 | 12.0 | Lung Ca. (non-s.cell) NCI-H23 | 11.7 | 28.5 |
| astrocytoma SW1783 | 8.1 | 38.2 | Lung Ca. (non-s.cell) HOP-62 | 5.3 | 24.0 |
| neuro*; met SK-N-AS | 14.2 | 6.5 | Lung Ca. (non-s.cl) NCI-H522 | 5.0 | 15.1 |
| astrocytoma SF-539 | 3.9 | 15.2 | Lung Ca. (squam.) SW 900 | 3.4 | 12.6 |
| astrocytoma SNB-75 | 8.8 | 11.3 | Lung Ca. (squam.) NCI-H596 | 1.5 | 1.9 |
| glioma SNB-19 | 4.1 | 20.0 | Mammary gland | 7.5 | 9.6 |
| glioma U251 | 2.5 | 5.8 | Breast ca.* (pl.ef) MCF-7 | 25.3 | 88.9 |
| glioma SF-295 | 3.4 | 24.0 | Breast ca.* (pl.ef) MDA-MB- | 21.8 | 6.4 |
| | | | 231 | | |
| Heart (Fetal) | 9.7 | 35.1 | Breast ca.* (pl.ef) T47D | 13.6 | 29.3 |
| Heart | 3.6 | 11.4 | Breast ca. BT-549 | 22.1 | 7.4 |
| Skeletal muscle (Fetal) | 8.2 | 44.1 | Breast Ca. MDA-N | 5.7 | 11.1 |
| Skeletal muscle | 5.6 | 47.6 | Ovary | 5.4 | 26.6 |
| Bone marrow | 3.2 | 1.7 | Ovarian Ca. OVCAR-3 | 2.5 | 4.8 |
| Thymus | 3.5 | 40.6 | Ovarian Ca. OVCAR-4 | 0.6 | 3.6 |
| Spleen | 5.4 | 10.9 | Ovarian Ca. OVCAR-5 | 3.8 | 13.1 |
| Lymph node | 2.8 | 4.4 | Ovarian Ca. OVCAR-8 | 5.9 | 21.2 |
| Colorectal | 1.9 | 9.4 | Ovarian Ca. IGROV-1 | 1.4 | 3.1 |
| Stomach | 2.2 | 2.7 | Ovarian Ca. (ascites) SK-OV-3 | 5.3 | 13.1 |
| Small intestine | 5.0 | 7.3 | Uterus | 3.9 | 6.0 |
| Colon Ca. SW480 | 6.0 | 12.6 | Placenta | 5.2 | 8.8 |
| Colon Ca.* SW620 (SW480 | 4.7 | 11.1 | Prostate | 2.0 | 6.7 |
| met) | | | | | |
| Colon Ca. HT29 | 2.6 | 7.1 | Prostate Ca.* (bone met) PC-3 | 5.4 | 9.7 |
| Colon Ca. HCT-116 | 9.5 | 22.4 | Testis | 7.7 | 24.8 |
| Colon Ca. CaCo-2 | 6.7 | 18.0 | Melanoma Hs688(A).T | 3.3 | 7.7 |
| CC Well to Mod Duff | 4.8 | 13.2 | Melanoma* (met) Hs688(B).T | 1.2 | 6.9 |
| (ODO3866) | | | | | |
| Colon Ca. HCC-2998 | 17.2 | 10.2 | Melanoma UACC-62 | 1.5 | 5.6 |
| Gastric ca. (liver met) NCI-N87 | 10.8 | 14.7 | Melanoma M14 | 4.3 | 8.1 |
| Bladder | 2.6 | 11.0 | Melanoma LOX IMVI | 4.8 | 2.9 |
| Trachea | 6.4 | 13.8 | Melanoma* (met) SK-MEL-5 | 6.9 | 10.4 |
| Kidney | 1.7 | 14.1 | Adipose | 2.3 | 6.0 |

TABLE GD
Panel 2D
Column A - Rel. Exp.(%) Ag2248, Run 159035545
Column B - Rel. Exp.(%) Ag2548, Run 162326203

| Tissue Name | A | B | Tissue Name | A | B |
|---|---|---|---|---|---|
| Normal Colon | 49.3 | 39.5 | Kidney Margin 8120608 | 4.7 | 6.3 |
| CC Well to Mod Diff (ODO3866) | 14.2 | 10.7 | Kidney Cancer 8120613 | 7.1 | 14.0 |
| CC Margin (ODO3866) | 10.7 | 8.9 | Kidney Margin 8120614 | 8.8 | 10.7 |
| CC Gr.2 rectosigmoid (ODO3868) | 6.5 | 5.9 | Kidney Cancer 9010320 | 10.9 | 13.7 |
| CC Margin (ODO3868) | 5.8 | 6.9 | Kidney Margin 9010321 | 9.7 | 18.4 |
| CC Mod Diff (ODO3920) | 38.4 | 21.5 | Normal Uterus | 6.5 | 8.0 |
| CC Margin (ODO3920) | 14.5 | 9.5 | Uterine Cancer 064011 | 42.9 | 24.1 |
| CC Gr.2 ascend colon (ODO3921) | 25.5 | 15.8 | Normal Thyroid | 40.3 | 31.0 |
| CC Margin (ODO3921) | 7.7 | 5.9 | Thyroid Cancer | 21.0 | 21.0 |
| CC from Partial Hepatectomy | 32.5 | 28.5 | Thyroid Cancer A302152 | 21.9 | 18.4 |
| (ODO4309) Mets | | | | | |
| Liver Margin (ODO4309) | 12.2 | 9.0 | Thyroid Margin A302153 | 37.9 | 39.0 |
| Colon mets to lung (ODO4451-01) | 15.6 | 8.5 | Normal Breast | 18.9 | 23.8 |
| Lung Margin (ODO4451-02) | 12.6 | 9.2 | Breast Cancer | 14.2 | 20.2 |
| Normal Prostate 6546-1 | 6.6 | 57.4 | Breast Cancer (ODO4590-01) | 100.0 | 100.0 |
| Prostate Cancer (ODO4410) | 40.3 | 30.1 | Breast Cancer Mets | 87.1 | 90.1 |
| | | | (ODO4590-03) | | |
| Prostate Margin (ODO4410) | 27.0 | 21.8 | Breast Cancer Metastasis | 37.6 | 37.4 |
| Prostate Cancer (ODO4720-01) | 28.5 | 18.3 | Breast Cancer | 14.6 | 14.1 |
| Prostate Margin (ODO4720-02) | 35.8 | 25.0 | Breast Cancer | 27.4 | 28.9 |
| Normal Lung | 56.6 | 39.0 | Breast Cancer 9100266 | 46.7 | 41.5 |
| Lung Met to Muscle (ODO4286) | 33.4 | 22.7 | Breast Margin 9100265 | 15.5 | 16.7 |
| Muscle Margin (ODO4286) | 22.1 | 12.3 | Breast Cancer A209073 | 42.3 | 42.9 |
| Lung Malignant Cancer | 33.4 | 27.0 | Breast Margin A209073 | 21.3 | 17.2 |
| (ODO3126) | | | | | |
| Lung Margin (ODO3126) | 27.5 | 21.9 | Normal Liver | 5.4 | 4.6 |
| Lung Cancer (ODO4404) | 13.3 | 14.9 | Liver Cancer | 3.8 | 3.0 |
| Lung Margin (ODO4404) | 12.0 | 11.6 | Liver Cancer 1025 | 4.2 | 2.3 |
| Lung Cancer (ODO4565) | 14.1 | 14.3 | Liver Cancer 1026 | 3.0 | 1.3 |
| Lung Margin (ODO4565) | 6.9 | 11.0 | Liver Cancer 6004-T | 3.6 | 1.6 |
| Lung Cancer (ODO4237-01) | 95.9 | 82.4 | Liver Tissue 6004-N | 11.7 | 9.0 |
| Lung Margin (ODO4237-02) | 15.5 | 13.7 | Liver Cancer 6005-T | 2.2 | 2.9 |
| Ocular Mel Met to Liver | 27.4 | 19.9 | Liver Tissue 6005-N | 4.4 | 3.8 |
| (ODO4310) | | | | | |
| Liver Margin (ODO4310) | 5.1 | 3.4 | Normal Bladder | 26.6 | 16.0 |
| Melanoma Metastasis | 24.8 | 18.8 | Bladder Cancer | 5.0 | 3.0 |
| Lung Margin (ODO4321) | 23.8 | 20.0 | Bladder Cancer | 17.1 | 8.8 |
| Normal Kidney | 40.1 | 48.0 | Bladder Cancer (ODO4718- | 22.2 | 15.5 |
| | | | 01) | | |
| Kidney Ca, Nuclear grade 2 | 30.6 | 41.8 | Bladder Normal Adjacent | 21.2 | 15.6 |
| (ODO4338) | | | (ODO4718-03) | | |
| Kidney Margin (ODO4338) | 16.4 | 15.9 | Normal Ovary | 12.6 | 8.8 |
| Kidney Ca Nuclear grade 1/2 | 11.3 | 15.8 | Ovarian Cancer | 21.6 | 16.5 |
| (ODO4339) | | | | | |
| Kidney Margin (ODO4339) | 19.6 | 24.5 | Ovarian Cancer (ODO4768- | 40.1 | 33.9 |
| | | | 07) | | |
| Kidney Ca, Clear cell type | 20.4 | 30.8 | Ovary Margin (ODO4768-08) | 11.3 | 4.0 |
| (ODO4340) | | | | | |
| Kidney Margin (ODO4340) | 18.4 | 13.9 | Normal Stomach | 12.2 | 8.8 |
| Kidney Ca, Nuclear grade 3 | 13.3 | 6.1 | Gastric Cancer 9060358 | 5.0 | 3.0 |
| (ODO4348) | | | | | |
| Kidney Margin (ODO4348) | 21.2 | 19.3 | Stomach Margin 9060359 | 16.0 | 11.0 |
| Kidney Cancer (ODO4622-01) | 19.3 | 19.2 | Gastric Cancer 9060395 | 16.3 | 12.6 |
| Kidney Margin (ODO4622-03) | 4.4 | 5.3 | Stomach Margin 9060394 | 13.9 | 10.6 |
| Kidney Cancer (ODO4450-01) | 23.8 | 27.0 | Gastric Cancer 9060397 | 24.0 | 12.1 |
| Kidney Margin (ODO4450-03) | 15.2 | 20.0 | Stomach Margin 9060396 | 6.4 | 7.1 |
| Kidney Cancer 8120607 | 5.6 | 4.2 | Gastric Cancer 064005 | 37.1 | 20.3 |

TABLE GE
Panel 3D
Column A - Rel. Exp.(%) Ag2548, Run 164886193

| Tissue Name | A | Tissue Name | A |
|---|---|---|---|
| 94905 Daoy | 8.7 | 94954 Ca Ski Cervical epidermoid | 10.6 |
| Medulloblastoma/Cerebellum | | carcinoma (metastasis | |
| 94906 TE671 | 10.7 | 94955 ES-2 Ovarian clear cell | 11.3 |
| Medulloblastom/Cerebellum | | carcinoma | |
| 94907 D283 Med | 40.6 | 94957 Ramos Stimulated with | 2.0 |
| Medulloblastoma/Cerebellum | | PMA/ionomycin 6 h | |
| 94908 PFSK-l Primitive | 9.0 | 94958 Ramos Stimulated with | 8.8 |
| Neuroectodermal/Cerebellum | | PMA/ionomycin 14 h | |
| 94909 XF-498 CNS | 9.3 | 94962 MEG-01 Chronic | 11.5 |
| | | myelogenous leukemia | |
| | | (megokaryoblast) | |
| 94910 SNB-78 CNS/glioma | 12.9 | 94963 Raji Burkitt's lymphoma | 4.5 |
| 94911 SF-268 CNS/glioblastoma | 9.4 | 94964 Daudi Burkitt's lymphoma | 12.0 |
| 94912 T98G Glioblastoma | 13.7 | 94965 U266 B-cell | 28.1 |
| | | plasmacytoma/myeloma | |
| 96776 SK-N-SH Neuroblastoma | 14.9 | 94968 CA46 Burkitt's lymphoma | 9.2 |
| (metastasis) | | | |
| 94913 SF-295 CNS/glioblastoma | 9.9 | 94970 RL non-Hodgkin's B-cell | 2.2 |
| | | lymphoma | |
| 94914 Cerebellum | 21.5 | 94972 JM1 pre-B-cell | 6.3 |
| | | lymphoma/leukemia | |
| 96777 Cerebellum | 6.0 | 94973 Jurkat T cell leukemia | 18.7 |
| 94916 NCI-H292 Mucoepidermoid | 25.7 | 94974 TF-l Erythroleukemia | 9.7 |
| lung carcinoma | | | |
| 94917 DMS-114 Small cell lung | 16.3 | 94975 HUT 78 T-cell lymphoma | 17.1 |
| cancer | | | |
| 94918 DMS-79 Small cell lung | 100.0 | 94977 U937 Histiocytic lymphoma | 11.2 |
| cancer/neuroendocrine | | | |
| 94919 NCI-H146 Small cell lung | 20.9 | 94980 KU-812 Myelogenous | 5.3 |
| cancer/neuroendocrine | | leukemia | |
| 94920 NCI-H526 Small cell lung | 36.6 | 769-P-Clear cell renal carcinoma | 6.2 |
| cancer/neuroendocrine | | | |
| 94921 NCI-N417 Small cell lung | 9.7 | 94983 Caki-2 Clear cell renal | 8.1 |
| cancer/neuroendocrine | | carcinoma | |
| 94923 NCI-H82 Small cell lung | 14.2 | 94984 SW 839 Clear cell renal | 2.9 |
| cancer/neuroendocrine | | carcinoma | |
| 94924 NCI-H157 Squamous cell lung | 19.6 | 94986 G401 Wilms' tumor | 8.8 |
| cancer (metastasis) | | | |
| 94925 NCI-H1155 Large cell lung | 34.6 | 94987 Hs766T Pancreatic carcinoma | 13.3 |
| cancer/neuroendocrine | | (LN metastasis) | |
| 94926 NCI-H1299 Large cell lung | 19.9 | 94988 CAPAN-1 Pancreatic | 7.7 |
| cancer/neuroendocrine | | adenocarcinoma (liver metastasis) | |
| 94927 NCI-H727 Lung carcinoid | 14.2 | 94989 SU86.86 Pancreatic carcinoma | 10.0 |
| | | (liver metastasis) | |
| 94928 NCI-UMC-11 Lung carcinoid | 12.6 | 94990 BxPC-3 Pancreatic | 4.3 |
| | | adenocarcinoma | |
| 94929 LX-1 Small cell lung cancer | 20.0 | 94991 HPAC Pancreatic | 6.6 |
| | | adenocarcinoma | |
| 94930 Colo-205 Colon cancer | 15.8 | 94992 MIA PaCa-2 Pancreatic | 4.6 |
| | | carcinoma | |
| 94931 KM12 Colon cancer | 9.3 | 94993 CFPAC-1 Pancreatic ductal | 19.5 |
| | | adenocarcinoma | |
| 94932 KM20L2 Colon cancer | 3.0 | 94994 PANC-1 Pancreatic epithelioid | 9.5 |
| | | ductal carcinoma | |
| 94933 NCI-H716 Colon cancer | 19.1 | 94996 T24 Bladder carcinma | 9.9 |
| | | (transitional cell | |
| 94935 SW-48 Colon adenocarcinoma | 7.9 | 5637- Bladder carcinoma | 4.7 |
| 94936 SW1116 Colon | 7.4 | 94998 HT-1197 Bladder carcinoma | 6.1 |
| adenocarcinoma | | | |
| 94937 LS 174T Colon | 4.6 | 94999 UM-UC-3 Bladder carcimia | 2.8 |
| adenocarcinoma | | (transitional cell) | |
| 94938 SW-948 Colon | 1.1 | 95000 A204 Rhabdomyosarcoma | 3.4 |
| adenocarcinoma | | | |
| 94939 SW-480 Colon | 2.7 | 95001 HT-1080 Fibrosarcoma | 10.7 |
| adenocarcinoma | | | |
| 94940 NCI-SNU-5 Gastric carcinoma | 9.3 | 95002 MG-63 Osteosarcoma (bone) | 1.3 |
| KATO III-Gastric carcinoma | 24.0 | 95003 SK-LMS-1 Leiomyosarcoma | 9.5 |
| | | (vulva) | |
| 94943 NCI-SNU-16 Gastric | 9.5 | 95004 SJRH30 Rhabdomyosarcoma | 10.2 |
| carcinoma | | (met to bone marrow) | |
| 94944 NCI-SNU-1 Gastric carcinoma | 12.2 | 95005 A431 Epidermoid carcinoma | 5.0 |
| 94946 RF-1 Gastric adenocarcinoma | 5.1 | 95007 WM266-4 Melanoma | 10.5 |
| 94947 RF-48 Gastric adenocarcinoma | 8.1 | DU 145-Prostate carcinoma (brain | 0.0 |
| | | metastasis) | |
| 96778 MKN-45 Gastric carcinoma | 5.3 | 95012 MDA-MB-468 Breast | 20.7 |
| | | adenocarcinoma | |
| 94949 NCI-N87 Gastric carcinoma | 7.4 | SCC-4-Squamous cell carcinoma of | 0.0 |
| | | tongue | |
| 94951 OVCAR-5 Ovarian carcinoma | 2.7 | SCC-9-Squamous cell carcinoma of | 0.0 |
| | | tongue | |
| 94952 RL95-2 Uterine carcinoma | 3.8 | SCC-15-Squamous cell carcinoma of | 0.0 |
| | | tongue | |
| 94953 HelaS3 Cervical | 10.7 | 95017 CAL 27 Squamous cell | 5.5 |
| adenocarcinoma | | carcinoma of tongue | |

TABLE GF
Panel 4D
Column A - Rel. Exp.(%) Ag2248, Run 159034717

| Tissue Name | A | Tissue Name | A |
|---|---|---|---|
| Secondary Th1 act | 27.2 | HUVEC IL-1 beta | 8.4 |
| Secondary Th2 act | 33.4 | HUVEC IFN gamma | 14.4 |
| Secondary Tr1 act | 37.4 | HUVEC TNF alpha + IFN gamma | 7.4 |
| Secondary Th1 rest | 11.7 | HUVEC TNF alpha + IL4 | 6.6 |
| Secondary Th2 rest | 10.4 | HUVEC IL-11 | 11.8 |
| Secondary Tr1 rest | 12.5 | Lung Microvascular EC none | 9.9 |
| Primary Th1 act | 28.5 | Lung Microvascular EC TNFalpha + IL- | 18.2 |
| | | 1 beta | |
| Primary Th2 act | 29.3 | Microvascular Dermal EC none | 28.9 |
| Primary Tr1 act | 29.3 | Microsvasular Dermal EC TNFalpha + IL- | 20.2 |
| | | 1 beta | |
| Primary Th1 rest | 62.4 | Bronchial epithelium TNFalpha + IL1 beta | 20.7 |
| Primary Th2 rest | 39.8 | Small airway epithelium none | 6.9 |
| Primary Tr1 rest | 15.3 | Small airway epithelium TNFalpha + IL- | 40.3 |
| | | 1 beta | |
| CD45RA CD4 lymphocyte act | 18.6 | Coronery artery SMC rest | 15.4 |
| CD45RO CD4 lymphocyte act | 20.9 | Coronery artery SMC TNFalpha + IL-1 beta | 6.8 |
| CD8 lymphocyte act | 14.7 | Astrocytes rest | 20.0 |
| Secondary CD8 lymphocyte rest | 11.9 | Astrocytes TNFalpha + IL-1 beta | 15.8 |
| Secondary CD8 lymphocyte act | 19.9 | KU-812 (Basophil) rest | 8.1 |
| CD4 lymphocyte none | 8.2 | KU-812 (Basophil) PMA/ionomycin | 20.2 |
| 2ry Th1/Th2/Tr1 anti-CD95 | 17.4 | CCD1106 (Keratinocytes) none | 11.5 |
| CH11 | | | |
| LAK cells rest | 19.2 | CCD1106 (Keratinocytes) TNFalpha + IL- | 5.3 |
| | | 1 beta | |
| LAK cells IL-2 | 18.2 | Liver cirrhosis | 2.4 |
| LAK cells IL-2 + IL-12 | 11.0 | Lupus kidney | 1.8 |
| LAK cells IL-2 + IFN gamma | 19.5 | NCI-H292 none | 39.5 |
| LAK cells IL-2 + IL-18 | 17.7 | NCI-H292 IL-4 | 38.2 |
| LAK cells PMA/ionomycin | 3.6 | NCI-H292 IL-9 | 40.1 |
| NK Cells IL-2 rest | 11.0 | NCI-H292 IL-13 | 18.2 |
| Two Way MLR 3 day | 19.2 | NCI-H292 IFN gamma | 14.7 |
| Two Way MLR 5 day | 8.9 | HPAEC none | 19.2 |
| Two Way MLR 7 day | 6.7 | HPAEC TNF alpha + IL-1 beta | 28.5 |
| PBMC rest | 5.8 | Lung fibroblast none | 17.4 |
| PBMC PWM | 40.6 | Lung fibroblast TNF alpha + IL-1 beta | 17.1 |
| PBMC PHA-L | 25.9 | Lung fibroblast IL-4 | 30.4 |
| Ramos (B cell) none | 26.6 | Lung fibroblast IL-9 | 20.2 |
| Ramos (B cell) ionomycin | 100.0 | Lung fibroblast IL-13 | 16.3 |
| B lymphocytes PWM | 35.6 | Lung fibroblast LFN gamma | 28.1 |
| B lymphocytes CD40L and IL-4 | 29.7 | Dermal fibroblast CCD1070 rest | 32.3 |
| EOL-1 dbcAMP | 10.5 | Dermal fibroblast CCD1070 TNF alpha | 57.0 |
| EOL-1 dbcAMP | 7.5 | Dermal fibroblast CCD1070 IL-1 beta | 15.3 |
| PMA/ionomycin | | | |
| Dendritic cells none | 11.6 | Dermal fibroblast IFN gamma | 12.1 |
| Dendritic cells LPS | 7.7 | Dermal fibroblast IL-4 | 20.3 |
| Dendritic cells anti-CD40 | 9.6 | IBD Colitis 2 | 2.3 |
| Monocytes rest | 12.6 | IBD Crohn's | 2.6 |
| Monocytes LPS | 21.0 | Colon | 11.5 |
| Macrophages rest | 24.5 | Lung | 16.2 |
| Macrophages LPS | 14.8 | Thymus | 38.7 |
| HUVEC none | 25.9 | Kidney | 71.2 |
| HUVEC starved | 40.9 | | |

TABLE GG
Panel 5 Islet
Column A - Rel. Exp.(%) Ag2248, Run 233070521

| Tissue Name | A | Tissue Name | A |
|---|---|---|---|
| 97457 Patient-02go adipose | 23.7 | 94709 Donor 2 AM - A adipose | 10.5 |
| 97476 Patient-07sk skeletal muscle | 12.3 | 94710 Donor 2 AM - B adipose | 5.0 |
| 97477 Patient-07ut uterus | 18.9 | 94711 Donor 2 AM - C adipose | 5.3 |
| 97478 Patient-07pl placenta | 35.6 | 94712 Donor 2 AD - A adipose | 14.5 |
| 99167 Bayer Patient 1 | 25.0 | 94713 Donor 2 AD - B adipose | 25.2 |
| 97482 Patient-08ut uterus | 23.2 | 94714 Donor 2 AD - C adipose | 18.7 |
| 97483 Patient-08pl placenta | 25.5 | 94742 Donor 3 U - A Mesenchymal | 7.4 |
| | | Stem Cells | |
| 97486 Patient-09sk skeletal muscle | 0.7 | 94743 Donor 3 U - B Mesenchymal | 11.2 |
| | | Stem Cells | |
| 97487 Patient-09ut uterus | 23.0 | 94730 Donor 3 AM - A adipose | 14.6 |
| 97488 Patient-09pl placenta | 15.3 | 94731 Donor 3 AM - B adipose | 4.5 |
| 97492 Patient-10ut uterus | 15.1 | 94732 Donor 3 AM - C adipose | 10.5 |
| 97493 Patient-10pl placenta | 52.9 | 94733 Donor 3 AD - A adipose | 40.1 |
| 97495 Patient-11go adipose | 9.1 | 94734 Donor 3 AD - B adipose | 16.0 |
| 97496 Patient-11sk skeletal muscle | 10.2 | 94735 Donor 3 AD - C adipose | 14.0 |
| 97497 Patient-11ut uterus | 21.8 | 77138 Liver HepG2untreated | 100.0 |
| 97498 Patient-11pl placenta | 36.3 | 73556 Heart Cardiac stromal cells | 9.5 |
| | | (primary) | |
| 97500 Patient-12go adipose | 21.0 | 81735 Small Intestine | 8.7 |
| 97501 Patient-12sk skeletal muscle | 35.4 | 72409 Kidney Proximal Convoluted | 16.7 |
| | | Tubule | |
| 97502 Patient-12ut uterus | 15.5 | 82685 Small intestine Duodenum | 5.1 |
| 97503 Patient-12p1 placenta | 9.5 | 90650 Adrenal Adrenocortical | 18.0 |
| | | adenoma | |
| 94721 Donor 2 U - A Mesenchymal | 8.3 | 72410 Kidney HRCE | 42.0 |
| Stem Cells | | | |
| 94722 Donor 2 U - B Mesenchymal | 8.3 | 72411 Kidney HRE | 27.0 |
| Stem Cells | | | |
| 94723 Donor 2 U - C Mesenchymal | 20.4 | 73139 Uterus Uterine smooth muscle | 41.5 |
| Stem Cells | | cells | |

Panel 1.3D Summary: Ag2248/Ag2548 Highest expression of this gene was detected seen in regions of the brain (CTs=28-29).

This gene encodes a protein that is homologous to steroid dehydrogenase. Steroid treatment is used in a number of clinical conditions including Alzheimer's disease (estrogen), treatment of symptoms associated with menopause (estrogen), multiple sclerosis (glucocorticoids), and spinal cord injury (methylprednisolone). Treatment with an antagonst of this gene product, or reduction of the levels of this gene product is useful for the inhibition of steroid degredation and for lowering the necessary amount given for a therapeutic effect, thus reducing peripheral side effects.

This gene was moderately expressed in a variety of metabolic tissues including pancreas, adrenal, thyroid, pituitary, adult and fetal heart, adult and fetal skeletal muscle, fetal liver, and adipose. This gene product is a small molecule drug target for the treatment of metabolic disease, including obesity and Types 1 and 2 diabetes.

The ubiquitous expression of this gene in this panel also showed that the protein encoded by this gene plays a role in cell survival and proliferation for a majority of cell types. There are significant levels of expression in the lung cancer cell line SHP-77. Expression of this gene is of use as a diagnostic marker for lung cancer. Modulation of the gene product is useful in the treatment of lung cancer

Panel 2D Summary: Ag2248/Ag2548 The highest level of expression was seen in a breast cancer sample (CTs=27-29). In addition, this gene was overexpressed in ovarian, gastric, breast, uterine, lung and colon cancers relative to the normal adjacent tissues from these patients. The expression of this gene is of use as a diagnostic marker for the presence of these cancers. Therapeutic inhibition of the activity of this gene product is effective in the treatment of these cancers.

Panel 3D Summary: Ag2548 This gene was expressed at a low to moderate level in most of the cells and tissues used in this panel, with highest expression in the small cell lung cancer cell line DMS-79 (CT=27.79). This ubiquitous expression showed that the gene product plays a role in cell survival and proliferation for a majority of cell types except cell lines derived from tongue squamous cell carcinoma.

Panel 4D Summary: Ag2248 This gene encodes a steroid dehydrogenase-like protein and was expressed at moderate levels (CT=28-32) in numerous immune cell types and tissues. Small molecule antagonists that block the function of the steroid dehydrogenase-like protein encoded by this gene are useful as therapeutics that reduce or eliminate the symptoms of patients suffering from autoimmune and inflammatory diseases such as asthma, allergies, inflammatory bowel disease, lupus erythematosus, or rheumatoid arthritis.

Panel 5 Islet Summary: Ag2248 The expression of this novel steroid dehydrogenase-like gene was highest in the liver HepG2 cell line, (CT=32.1). Lower but still significant levels of expression were seen in several placenta samples, uterine smooth muscle, adipose samples, differentiated mesenchymal stem cells, and kidney and skeletal muscle from a diabetic patient. Expression in liver cells and placenta showed that the role of this novel steroid dehydrogenase is similar to the role of other steroid dehydrogenases which are involved in steroid and bile acid metabolism. Very low expression of this gene was also seen in a human pancreatic islet sample. Therefore, small molecule therapeutics against this gene product are effective in disorders in which expression of this gene is dysregulated.

H. CG50315-01: Olfactory Receptor

Expression of full-length physical clone CG50315-01 was assessed using the primer-probe sets Ag1665 and Ag2542, described in Tables HA and HB. Results of the RTQ-PCR runs are shown in Tables HC, HD, and HE.

TABLE HA
Probe Name Ag1665

| | | | Start | SEQ ID |
|---|---|---|---|---|
| Primers | Sequences | Length | Position | No |
| Forward | 5′-atcttctttggcaattttgtga-3′ | 22 | 332 | 1157 |
| Probe | TET-5′-aatgcctccgtcatcctgatttcct-3′- | 25 | 299 | 1158 |
| | TAMRA | | | |
| Reverse | 5′-atggtcttgatgatgagcagat-3′ | 22 | 277 | 1159 |

TABLE HB
Probe Name Ag2542

| | | | Start | SEQ ID |
|---|---|---|---|---|
| Primers | Sequences | Length | Position | No |
| Forward | 5′-ggaatgtggggatgattatgtt-3′ | 22 | 808 | 1160 |
| Probe | TET-5′-tccaagtagatgtcaaactctacacccca- | 29 | 777 | 1161 |
| | 3′-TAMRA | | | |
| Reverse | 5′-gaggtggctcaggaagaagtac-3′ | 22 | 753 | 1162 |

TABLE HC
PGI1.0
Column A - Rel. Exp.(%) Ag2542, Run 395549579

| Tissue Name | A | Tissue Name | A |
|---|---|---|---|
| 162191 Normal Lung 1 (IBS) | 0.2 | 162185 Emphysema Lung 12 (Ardais) | 2.4 |
| 160468 MD lung | 1.9 | 162184 Emphysema Lung 13 (Ardais) | 1.5 |
| 156629 MD Lung 13 | 2.7 | 162183 Emphysema Lung 14 (Ardais) | 2.0 |
| 162570 Normal Lung 4 (Aastrand) | 1.6 | 162188 Emphysema Lung 15 (Genomic | 41.2 |
| | | Collaborative) | |
| 162571 Normal Lung 3 (Aastrand) | 0.5 | 162177 NAT UC Colon 1(Ardais) | 0.0 |
| 162187 Fibrosis Lung 2 (Genomic | 100.0 | 162176 UC Colon 1(Ardais) | 0.5 |
| Collaborative) | | | |
| 151281 Fibrosis lung 11(Ardais) | 3.0 | 162179 NAT UC Colon 2(Ardais) | 1.6 |
| 162186 Fibrosis Lung 1 (Genomic | 34.6 | 162178 UC Colon 2(Ardais) | 0.0 |
| Collaborative) | | | |
| 162190 Asthma Lung 4 (Genomic | 70.7 | 162181 NAT UC Colon 3(Ardais) | 1.7 |
| Collaborative) | | | |
| 160467 Asthma Lung 13 (MD) | 1.5 | 162180 UC Colon 3(Ardais) | 0.0 |
| 137027 Emphysema Lung 1 | 0.0 | 162182 NAT UC Colon 4 (Ardais) | 1.2 |
| (Ardais) | | | |
| 137028 Emphysema Lung 2 | 0.0 | 137042 UC Colon 1108 | 1.3 |
| (Ardais) | | | |
| 137040 Emphysema Lung 3 | 1.7 | 137029 UC Colon 8215 | 1.1 |
| (Ardais) | | | |
| 137041 Emphysema Lung 4 | 5.4 | 137031 UC Colon 8217 | 1.6 |
| (Ardais) | | | |
| 137043 Emphysema Lung 5 | 6.0 | 137036 UC Colon 1137 | 0.0 |
| (Ardais) | | | |
| 142817 Emphysema Lung 6 | 3.5 | 137038 UC Colon 1491 | 2.4 |
| (Ardais) | | | |
| 142818 Emphysema Lung 7 | 6.3 | 137039 UC Colon 1546 | 2.8 |
| (Ardais) | | | |
| 142819 Emphysema Lung 8 | 4.6 | 162593 Crohn's 47751 (NDRI) | 0.0 |
| (Ardais) | | | |
| 142820 Emphysema Lung 9 | 3.9 | 162594 NAT Crohn's 47751 (NDRI) | 0.0 |
| (Ardais) | | | |
| 142821 Emphysema Lung 10 | 6.5 | | |
| (Ardais) | | | |

TABLE HD
Panel 2D
Column A - Rel. Exp.(%) Ag2542, Run 165910338

| Tissue Name | A | Tissue Name | A |
|---|---|---|---|
| Normal Colon | 0.0 | Kidney Margin 8120608 | 5.5 |
| CC Well to Mod Diff (ODO3866) | 5.1 | Kidney Cancer 8120613 | 0.0 |
| CC Margin (ODO3866) | 0.0 | Kidney Margin 8120614 | 5.9 |
| CC Gr.2 rectosigmoid (ODO3868) | 0.0 | Kidney Cancer 9010320 | 0.0 |
| CC Margin (ODO3868) | 0.0 | Kidney Margin 9010321 | 5.1 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon (ODO3921) | 0.0 | Normal Thyroid | 0.0 |
| CC Margin (ODO3921) | 9.9 | Thyroid Cancer | 8.1 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 0.0 | Normal Breast | 0.0 |
| Lung Margin (OD04451-02) | 4.0 | Breast Cancer | 0.0 |
| Normal Prostate 6546-1 | 0.0 | Breast Cancer (OD04590-01) | 0.0 |
| Prostate Cancer (OD04410) | 0.0 | Breast Cancer Mets (0D04590-03) | 9.3 |
| Prostate Margin (OD04410) | 0.0 | Breast Cancer Metastasis | 4.8 |
| Prostate Cancer (OD04720-01) | 0.0 | Breast Cancer | 0.0 |
| Prostate Margin (OD04720-02) | 0.0 | Breast Cancer | 0.0 |
| Normal Lung | 0.0 | Breast Cancer 9100266 | 0.0 |
| Lung Met to Muscle (ODO4286) | 5.1 | Breast Margin 9100265 | 0.0 |
| Muscle Margin (ODO4286) | 0.0 | Breast Cancer A209073 | 3.1 |
| Lung Malignant Cancer (OD03126) | 0.0 | Breast Margin A209073 | 0.0 |
| Lung Margin (OD03126) | 0.0 | Normal Liver | 0.0 |
| Lung Cancer (OD04404) | 22.8 | Liver Cancer | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 7.7 |
| Lung Margin (OD04237-02) | 0.0 | Liver Cancer 6005-T | 0.0 |
| Ocular Mel Met to Liver (ODO4310) | 7.9 | Liver Tissue 6005-N | 0.0 |
| Liver Margin (OD04310) | 0.0 | Normal Bladder | 15.7 |
| Melanoma Metastasis | 0.0 | Bladder Cancer | 0.0 |
| Lung Margin (OD04321) | 0.0 | Bladder Cancer | 12.6 |
| Normal Kidney | 100.0 | Bladder Cancer (OD04718-01) | 0.0 |
| Kidney Ca, Nuclear grade 2 (OD04338) | 0.0 | Bladder Normal Adjacent | 0.0 |
| | | (OD04718-03) | |
| Kidney Margin (OD04338) | 0.0 | Normal Ovary | 4.8 |
| Kidney Ca Nuclear grade 1/2 (OD04339) | 16.6 | Ovarian Cancer | 0.0 |
| Kidney Margin (OD04339) | 37.6 | Ovarian Cancer (OD04768-07) | 0.0 |
| Kidney Ca, Clear cell type (OD04340) | 11.2 | Ovary Margin (OD04768-08) | 0.0 |
| Kidney Margin (OD04340) | 4.8 | Normal Stomach | 0.0 |
| Kidney Ca, Nuclear grade 3 (OD04348) | 0.0 | Gastric Cancer 9060358 | 0.0 |
| Kidney Margin (OD04348) | 3.9 | Stomach Margin 9060359 | 0.0 |
| Kidney Cancer (OD04622-01) | 0.0 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 0.0 |
| Kidney Margin (OD04450-03) | 11.2 | Stomach Margin 9060396 | 0.0 |
| Kidney Cancer 8120607 | 0.0 | Gastric Cancer 064005 | 8.7 |

TABLE HE
Panel 4D
Column A - Rel. Exp.(%) Ag1665, Run 165974950
Column B - Rel. Exp.(%) Ag1665, Run 165974996
Column C - Rel. Exp.(%) Ag2542, Run 164392485
Column D - Rel. Exp.(%) Ag2542, Run 165871865

| Tissue Name | A | B | C | D |
|---|---|---|---|---|
| Secondary Th1 act | 0.0 | 0.0 | 0.0 | 0.0 |
| Secondary Th2 act | 0.0 | 0.0 | 0.0 | 0.0 |
| Secondary Tr1 act | 3.5 | 4.2 | 0.0 | 0.0 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.0 | 0.0 | 0.0 |
| Primary Tr1 act | 0.0 | 7.3 | 0.0 | 0.0 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 8.4 | 0.0 | 0.0 |
| CD45RA CD4 lymphocyte act | 0.0 | 14.3 | 0.0 | 0.0 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 5.4 | 0.0 | 0.0 |
| Secondary CD8 lymphocyte act | 0.0 | 13.3 | 0.0 | 0.0 |
| CD4 lymphocyte none | 0.0 | 0.0 | 0.0 | 0.0 |
| 2ry Th1/Th2/Tr1 anti-CD95 CH11 | 0.0 | 9.0 | 0.0 | 0.0 |
| LAK cells rest | 0.0 | 0.0 | 0.0 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.0 |
| NK Cells IL-2 rest | 0.0 | 0.0 | 0.0 | 0.0 |
| Two Way MLR 3 day | 0.0 | 7.3 | 0.0 | 0.0 |
| Two Way MLR 5 day | 0.0 | 20.2 | 0.0 | 0.0 |
| Two Way MLR 7 day | 0.0 | 0.0 | 0.0 | 0.0 |
| PBMC rest | 0.0 | 0.0 | 0.1 | 0.0 |
| PBMC PWM | 0.0 | 0.0 | 0.0 | 0.0 |
| PBMC PHA-L | 3.1 | 0.0 | 0.0 | 0.0 |
| Ramos (B cell) none | 0.0 | 6.5 | 0.0 | 0.0 |
| Ramos (B cell) ionomycin | 0.0 | 49.3 | 0.0 | 0.0 |
| B lymphocytes PWM | 0.0 | 0.0 | 0.0 | 6.3 |
| B lymphocytes CD40L and IL-4 | 0.0 | 0.0 | 0.0 | 0.0 |
| EOL-1 dbcAMP | 0.0 | 0.0 | 0.0 | 0.0 |
| EOL-1 dbcAMP PMA/ionomycin | 0.0 | 0.0 | 0.0 | 0.0 |
| Dendritic cells none | 0.0 | 7.5 | 0.0 | 0.0 |
| Dendritic cells LPS | 0.0 | 0.0 | 0.0 | 0.0 |
| Dendritic cells anti-CD40 | 0.0 | 0.0 | 0.0 | 0.0 |
| Monocytes rest | 0.0 | 0.0 | 0.0 | 0.0 |
| Monocytes LPS | 0.0 | 0.0 | 0.0 | 0.0 |
| Macrophages rest | 0.0 | 14.4 | 0.0 | 0.0 |
| Macrophages LPS | 0.0 | 12.6 | 0.0 | 0.0 |
| HUVEC none | 0.0 | 0.0 | 7.6 | 0.0 |
| HUVEC starved | 0.0 | 0.0 | 0.0 | 0.0 |
| HUVEC IL-1 beta | 0.0 | 0.0 | 0.0 | 10.4 |
| HUVEC IFN gamma | 0.0 | 0.0 | 0.0 | 0.0 |
| HUVEC TNF alpha + IFN gamma | 0.0 | 0.0 | 0.0 | 0.0 |
| HUVEC TNF alpha + IL4 | 0.0 | 0.0 | 0.0 | 0.0 |
| HUVEC IL-11 | 0.0 | 0.0 | 0.0 | 0.0 |
| Lung Microvascular EC none | 0.0 | 0.0 | 0.0 | 1.5 |
| Lung Microvascular EC TNFalpha + | 0.0 | 0.0 | 0.0 | 0.0 |
| IL-1 beta | | | | |
| Microvascular Dermal EC none | 0.0 | 10.5 | 0.0 | 0.0 |
| Microsvasular Dermal EC TNFalpha + | 0.0 | 0.0 | 0.0 | 0.0 |
| IL-1 beta | | | | |
| Bronchial epithelium TNFalpha + IL1 beta | 3.5 | 0.0 | 0.0 | 23.0 |
| Small airway epithelium none | 2.9 | 4.4 | 100.0 | 0.0 |
| Small airway epithelium TNFalpha + | 6.2 | 9.2 | 0.1 | 25.2 |
| IL-1 beta | | | | |
| Coronery artery SMC rest | 0.0 | 20.4 | 0.0 | 0.0 |
| Coronery artery SMC TNFalpha + IL-1 beta | 2.9 | 8.2 | 0.1 | 13.0 |
| Astrocytes rest | 0.0 | 0.0 | 0.0 | 0.0 |
| Astrocytes TNFalpha + IL-1 beta | 0.0 | 0.0 | 0.0 | 0.0 |
| KU-812 (Basophil) rest | 0.0 | 0.0 | 0.0 | 0.0 |
| KU-812 (Basophil) PMA/ionomycin | 0.0 | 0.0 | 0.0 | 0.0 |
| CCD1106 (Keratinocytes) none | 0.0 | 0.0 | 0.0 | 0.0 |
| CCD1106 (Keratinocytes) TNFalpha + | 7.1 | 0.0 | 0.0 | 7.8 |
| IL-1 beta | | | | |
| Liver cirrhosis | 100.0 | 100.0 | 0.2 | 100.0 |
| Lupus kidney | 3.0 | 0.0 | 0.0 | 4.0 |
| NCI-H292 none | 0.0 | 0.0 | 0.0 | 0.0 |
| NCI-H292 IL-4 | 0.0 | 4.8 | 0.0 | 0.0 |
| NCI-H292 IL-9 | 0.0 | 0.0 | 0.0 | 5.7 |
| NCI-H292 IL-13 | 0.0 | 0.0 | 0.0 | 0.0 |
| NCI-H292 IFN gamma | 0.0 | 0.0 | 0.0 | 0.0 |
| HPAEC none | 0.0 | 0.0 | 0.0 | 0.0 |
| HPAEC TNF alpha + IL-1 beta | 0.0 | 0.0 | 0.0 | 0.0 |
| Lung fibroblast none | 0.0 | 0.0 | 0.0 | 0.0 |
| Lung fibroblast TNF alpha + IL-1 beta | 2.1 | 0.0 | 0.0 | 0.0 |
| Lung fibroblast IL-4 | 0.0 | 0.0 | 0.0 | 0.0 |
| Lung fibroblast IL-9 | 0.0 | 0.0 | 0.0 | 4.3 |
| Lung fibroblast IL-13 | 0.0 | 0.0 | 0.0 | 0.0 |
| Lung fibroblast IFN gamma | 0.0 | 0.0 | 0.0 | 0.0 |
| Dermal fibroblast CCD1070 rest | 0.0 | 0.0 | 0.0 | 6.2 |
| Dermal fibroblast CCD1070 TNF alpha | 0.0 | 0.0 | 0.0 | 0.0 |
| Dermal fibroblast CCD1070 IL-1 beta | 0.0 | 0.0 | 0.0 | 0.0 |
| Dermal fibroblast IFN gamma | 0.0 | 0.0 | 0.0 | 0.0 |
| Dermal fibroblast IL-4 | 0.0 | 0.0 | 0.0 | 0.0 |
| IBD Colitis 2 | 20.2 | 38.7 | 0.0 | 16.8 |
| IBD Crohn's | 4.0 | 16.8 | 0.0 | 0.0 |
| Colon | 2.8 | 12.9 | 0.0 | 10.4 |
| Lung | 3.8 | 0.0 | 0.0 | 4.7 |
| Thymus | 20.6 | 35.6 | 0.2 | 27.0 |
| Kidney | 0.0 | 0.0 | 0.0 | 0.0 |

PGI1.0 Summary: Ag2542 Expression of this gene was highest in a lung fibrosis sample (CT=31). Expression of this gene was also significantly upregulated in another lung fibrosis sample as well as an asthmatic lung sample; more modest overexpression was seen in lung samples from patients with emphysema. Thus, gene or protein levels are useful for the detection of lung diseases such as lung fibrosis, emphysema and asthma. Furthermore, therapeutic modulation of the activity of this gene or its protein product is useful in the treatment of lung fibrosis, emphysema or asthma.

Panel 2D Summary: Ag1665 The expression of this gene was low in the samples on Panel 2D. The highest expression was associated with a sample of normal kidney (CT=32.9). In addition, there was a cluster of expression associated with normal kidney tissue when compared to malignant kidney tissue. Thus, the loss of expression of this gene was associated with kidney cancer, and as such, therapeutic application of the protein or its replacement by gene therapy is of use in the treatment of kidney cancer.

Panel 4D Summary: Ag1665/Ag2542 Mmoderate expression of this gene was seen in one IBD colitis sample, with lower expression in a second colitis sample in 3 (of 4 possible) experiments. In addition, low expression was detected in liver cirrhosis (CT=32.7) and thymus (CT=35) in 3 (of 4 possible) determinations. The function of the GPCR encoded by this gene is important in the disease processes in both inflammatory bowel disease and in liver cirrhosis. Therefore, blocking antibodies or small molecule antagonists targeted to this GPCR are useful as therapeutics in colitis and in cirrhosis.

I. CG50341-01: GPCR

Expression of gene CG50341-01 was assessed using the primer-probe set Ag1201, described in Table IA. Results of the RTQ-PCR runs are shown in Tables IB, IC and ID.

TABLE IA
Probe Name Ag1201

| | | | Start | SEQ ID |
|---|---|---|---|---|
| Primers | Sequences | Length | Position | No |
| Forward | 5′-agagacaatccaaagccttttc-3′ | 22 | 709 | 1163 |
| Probe | TET-5′-caactgtgtgcctcacctcattgttg-3′- | 26 | 731 | 1164 |
| | TAMRA | | | |
| Reverse | 5′-agaccctggctttaaataagca-3′ | 22 | 785 | 1165 |

TABLE IB
Panel 1.3D
Column A - Rel. Exp.(%) Ag1201, Run 147490505
Column B - Rel. Exp.(%) Ag1201, Run 152628232
Column C - Rel. Exp.(%) Ag1201, Run 165528215

| Tissue Name | A | B | C | Tissue Name | A | B | C |
|---|---|---|---|---|---|---|---|
| Liver adenocarcinoma | 0.0 | 13.7 | 0.0 | Kidney (fetal) | 15.7 | 19.6 | 0.0 |
| Pancreas | 0.0 | 0.0 | 0.0 | Renal Ca. 786-0 | 0.0 | 0.0 | 0.0 |
| Pancreatic ca. CAPAN 2 | 0.0 | 0.0 | 66.9 | Renal ca. A498 | 0.0 | 0.0 | 0.0 |
| Adrenal gland | 0.0 | 0.0 | 0.0 | Renal ca. RXF 393 | 0.0 | 0.0 | 0.0 |
| Thyroid | 0.0 | 26.2 | 0.0 | Renal ca. ACHN | 0.0 | 0.0 | 0.0 |
| Salivary gland | 0.0 | 14.7 | 0.0 | Renal ca. UO-31 | 0.0 | 0.0 | 0.0 |
| Pituitary gland | 7.5 | 0.0 | 0.0 | Renal ca. TK-10 | 0.0 | 0.0 | 0.0 |
| Brain (fetal) | 0.0 | 0.0 | 0.0 | Liver | 0.0 | 0.0 | 0.0 |
| Brain (whole) | 3.3 | 0.0 | 0.0 | Liver (fetal) | 0.0 | 0.0 | 0.0 |
| Brain (amygdala) | 0.0 | 0.0 | 0.0 | Liver ca. (hepatoblast) | 0.0 | 0.0 | 0.0 |
| | | | | HepG2 | | | |
| Brain (cerebellum) | 0.0 | 0.0 | 21.0 | Lung | 9.2 | 0.0 | 0.0 |
| Brain (hippocampus) | 0.0 | 0.0 | 0.0 | Lung (fetal) | 0.0 | 0.0 | 11.4 |
| Brain (substantia nigra) | 0.0 | 0.0 | 0.0 | Lung ca. (small cell) LX- | 0.0 | 0.0 | 0.0 |
| | | | | 1 | | | |
| Brain (thalamus) | 0.0 | 0.0 | 0.0 | Lung ca. (small cell) NCI- | 0.0 | 0.0 | 0.0 |
| Cerebral Cortex | 0.0 | 0.0 | 0.0 | Lung ca. (s.cell var.) | 0.0 | 0.0 | 0.0 |
| | | | | SHP-77 | | | |
| Spinal cord | 8.3 | 0.0 | 21.2 | Lung ca. (large cell) NCI- | 0.0 | 0.0 | 0.0 |
| | | | | H460 | | | |
| glio/astro U87-MG | 0.0 | 0.0 | 0.0 | Lung ca. (non-sm. cell) | 1.5 | 0.0 | 0.0 |
| | | | | A549 | | | |
| glio/astro U-118-MG | 0.0 | 0.0 | 0.0 | Lung Ca. (non-s.cell) | 6.2 | 94.0 | 0.0 |
| | | | | NCI-H23 | | | |
| astrocytoma SW1783 | 0.0 | 0.0 | 0.0 | Lung Ca. (non-s.cell) | 20.9 | 27.0 | 17.9 |
| | | | | HOP-62 | | | |
| neuro*; met SK-N-AS | 0.0 | 0.0 | 0.0 | Lung Ca. (non-s.cl) NCI- | 0.0 | 0.0 | 0.0 |
| | | | | H522 | | | |
| astrocytoma SF-539 | 0.0 | 15.6 | 0.0 | Lung ca. (squam.) SW | 0.0 | 0.0 | 0.0 |
| | | | | 900 | | | |
| astrocytoma SNB-75 | 0.0 | 0.0 | 0.0 | Lung Ca. (squam.) NCI- | 0.0 | 0.0 | 0.0 |
| | | | | H596 | | | |
| glioma SNB-19 | 0.0 | 12.0 | 0.0 | Mammary gland | 15.6 | 12.9 | 0.0 |
| glioma U251 | 0.0 | 0.0 | 27.0 | Breast ca.* (pl.ef) MCF-7 | 0.0 | 0.0 | 0.0 |
| glioma SF-295 | 0.0 | 0.0 | 0.0 | Breast ca.* (pl.ef) MDA- | 0.0 | 0.0 | 0.0 |
| | | | | MB-231 | | | |
| Heart (Fetal) | 0.0 | 0.0 | 0.0 | Breast ca.* (pl.ef) T47D | 0.0 | 0.0 | 0.0 |
| Heart | 7.7 | 0.0 | 0.0 | Breast Ca. BT-549 | 6.7 | 0.0 | 0.0 |
| Skeletal muscle (Fetal) | 11.2 | 10.7 | 0.0 | Breast Ca. MDA-N | 0.0 | 0.0 | 0.0 |
| Skeletal muscle | 0.0 | 0.0 | 0.0 | Ovary | 0.0 | 0.0 | 0.0 |
| Bone marrow | 0.0 | 20.7 | 15.5 | Ovarian ca. OVCAR-3 | 6.2 | 0.0 | 0.0 |
| Thymus | 8.6 | 0.0 | 0.0 | Ovarian ca. OVCAR-4 | 0.0 | 0.0 | 0.0 |
| Spleen | 11.3 | 28.1 | 14.0 | Ovarian ca. OVCAR-5 | 0.0 | 0.0 | 0.0 |
| Lymph node | 7.4 | 0.0 | 17.3 | Ovarian ca. OVCAR-8 | 33.7 | 83.5 | 28.1 |
| Colorectal | 10.8 | 0.0 | 0.0 | Ovarian ca. IGROV-1 | 0.0 | 15.3 | 0.0 |
| Stomach | 5.9 | 12.4 | 33.9 | Ovarian ca. (ascites) SK- | 0.0 | 0.0 | 0.0 |
| | | | | OV-3 | | | |
| Small intestine | 0.0 | 11.7 | 0.0 | Uterus | 0.0 | 0.0 | 0.0 |
| Colon ca. SW480 | 0.0 | 0.0 | 0.0 | Placenta | 0.0 | 0.0 | 0.0 |
| Colon ca.* SW620 (SW480 | 0.0 | 0.0 | 0.0 | Prostate | 0.0 | 22.2 | 30.1 |
| met) | | | | | | | |
| Colon ca. HT29 | 0.0 | 0.0 | 0.0 | Prostate ca.* (bone met) | 14.4 | 19.5 | 85.3 |
| | | | | PC-3 | | | |
| Colon ca. HCT-116 | 0.0 | 0.0 | 0.0 | Testis | 100.0 | 100.0 | 100.0 |
| Colon ca. CaCo-2 | 0.0 | 0.0 | 0.0 | Melanoma Hs688(A).T | 0.0 | 0.0 | 0.0 |
| CC Well to Mod Duff | 0.0 | 0.0 | 0.0 | Melanoma* (met) | 6.9 | 0.0 | 0.0 |
| (ODO3866) | | | | Hs688(B).T | | | |
| Colon ca. HCC-2998 | 0.0 | 0.0 | 0.0 | Melanoma UACC-62 | 0.0 | 0.0 | 0.0 |
| Gastric ca. (liver met) NCI- | 0.0 | 0.0 | 0.0 | Melanoma M14 | 0.0 | 0.0 | 0.0 |
| N87 | | | | | | | |
| Bladder | 0.0 | 0.0 | 0.0 | Melanoma LOX IMVI | 0.0 | 0.0 | 11.3 |
| Trachea | 71.2 | 0.0 | 0.0 | Melanoma* (met) SK- | 0.0 | 0.0 | 0.0 |
| | | | | MEL-5 | | | |
| Kidney | 0.0 | 0.0 | 0.0 | Adipose | 0.0 | 12.0 | 0.0 |

TABLE IC
Panel 2D
Column A - Rel. Exp.(%) Ag1201, Run 147490530
Column B - Rel. Exp.(%) Ag1201, Run 148032505

| Tissue Name | A | B | Tissue Name | A | B |
|---|---|---|---|---|---|
| Normal Colon | 14.2 | 12.9 | Kidney Margin 8120608 | 0.0 | 0.0 |
| CC Well to Mod Diff (ODO3866) | 10.1 | 14.6 | Kidney Cancer 8120613 | 0.0 | 4.0 |
| CC Margin (ODO3866) | 0.0 | 10.7 | Kidney Margin 8120614 | 0.0 | 0.0 |
| CC Gr.2 rectosigmoid (ODO3868) | 0.0 | 4.5 | Kidney Cancer 9010320 | 0.0 | 0.0 |
| CC Margin (ODO3868) | 0.0 | 4.9 | Kidney Margin 9010321 | 0.0 | 0.0 |
| CC Mod Diff (ODO3920) | 0.1 | 5.4 | Normal Uterus | 0.0 | 3.6 |
| CC Margin (ODO3920) | 13.4 | 5.6 | Uterine Cancer 064011 | 66.4 | 54.0 |
| CC Gr.2 ascend colon (ODO3921) | 4.0 | 3.3 | Normal Thyroid | 9.5 | 0.0 |
| CC Margin (ODO3921) | 5.1 | 18.7 | Thyroid Cancer | 0.0 | 3.4 |
| CC from Partial Hepatectomy | 0.0 | 0.0 | Thyroid Cancer A302152 | 11.9 | 33.0 |
| (ODO4309) Mets | | | | | |
| Liver Margin (ODO4309) | 0.0 | 0.0 | Thyroid Margin A302153 | 28.5 | 39.5 |
| Colon mets to lung (ODO4451-01) | 4.3 | 0.0 | Normal Breast | 18.2 | 28.3 |
| Lung Margin (ODO4451-02) | 5.0 | 0.0 | Breast Cancer | 42.3 | 65.5 |
| Normal Prostate 6546-1 | 0.0 | 12.8 | Breast Cancer (ODO4590-01) | 0.0 | 0.0 |
| Prostate Cancer (ODO4410) | 100.0 | 100.0 | Breast Cancer Mets | 0.0 | 0.0 |
| | | | (ODO4590-03) | | |
| Prostate Margin (ODO4410) | 8.0 | 9.6 | Breast Cancer Metastasis | 31.6 | 35.6 |
| Prostate Cancer (ODO4720-01) | 50.0 | 64.2 | Breast Cancer | 18.4 | 28.7 |
| Prostate Margin (ODO4720-02) | 47.0 | 29.7 | Breast Cancer | 37.6 | 35.8 |
| Normal Lung | 2.8 | 10.6 | Breast Cancer 9100266 | 31.4 | 36.9 |
| Lung Met to Muscle (ODO4286) | 3.9 | 11.1 | Breast Margin 9100265 | 14.5 | 48.3 |
| Muscle Margin (ODO4286) | 0.0 | 0.0 | Breast Cancer A209073 | 51.8 | 68.3 |
| Lung Malignant Cancer | 0.0 | 0.0 | Breast Margin A209073 | 41.5 | 22.7 |
| (ODO3126) | | | | | |
| Lung Margin (ODO3126) | 0.0 | 5.2 | Normal Liver | 13.5 | 0.0 |
| Lung Cancer (ODO4404) | 28.5 | 6.0 | Liver Cancer | 0.0 | 0.0 |
| Lung Margin (ODO4404) | 5.4 | 19.2 | Liver Cancer 1025 | 4.7 | 5.0 |
| Lung Cancer (ODO4565) | 0.0 | 0.0 | Liver Cancer 1026 | 0.0 | 0.0 |
| Lung Margin (ODO4565) | 1.8 | 0.0 | Liver Cancer 6004-T | 0.0 | 3.8 |
| Lung Cancer (ODO4237-01) | 0.0 | 0.0 | Liver Tissue 6004-N | 3.6 | 0.0 |
| Lung Margin (ODO4237-02) | 6.2 | 4.9 | Liver Cancer 6005-T | 0.0 | 0.0 |
| Ocular Mel Met to Liver | 0.0 | 10.4 | Liver Tissue 6005-N | 0.0 | 0.0 |
| (ODO4310) | | | | | |
| Liver Margin (ODO4310) | 2.5 | 3.5 | Normal Bladder | 9.8 | 11.2 |
| Melanoma Metastasis | 0.0 | 0.0 | Bladder Cancer | 0.0 | 0.0 |
| Lung Margin (ODO4321) | 0.0 | 0.0 | Bladder Cancer | 5.5 | 24.1 |
| Normal Kidney | 6.3 | 11.7 | Bladder Cancer (ODO4718- | 0.0 | 0.0 |
| | | | 01) | | |
| Kidney Ca, Nuclear grade 2 | 4.7 | 13.1 | Bladder Normal Adjacent | 5.0 | 13.6 |
| (ODO4338) | | | (ODO4718-03) | | |
| Kidney Margin (ODO4338) | 29.5 | 94.6 | Normal Ovary | 20.6 | 0.0 |
| Kidney Ca Nuclear grade 1/2 | 29.5 | 94.6 | Ovarian Cancer | 92.7 | 99.3 |
| (ODO4339) | | | | | |
| Kidney Margin (ODO4339) | 0.0 | 17.1 | Ovarian Cancer (ODO4768- | 0.0 | 9.5 |
| | | | 07) | | |
| Kidney Ca, Clear cell type | 3.6 | 0.0 | Ovary Margin (ODO4768-08) | 0.0 | 5.3 |
| (ODO4340) | | | | | |
| Kidney Margin (ODO4340) | 0.0 | 0.0 | Normal Stomach | 0.0 | 0.0 |
| Kidney Ca, Nuclear grade 3 | 7.9 | 5.1 | Gastric Cancer 9060358 | 0.0 | 0.0 |
| (ODO4348) | | | | | |
| Kidney Margin (ODO4348) | 7.4 | 0.0 | Stomach Margin 9060359 | 10.4 | 0.0 |
| Kidney Cancer (ODO4622-01) | 0.0 | 0.0 | Gastric Cancer 9060395 | 2.6 | 0.0 |
| Kidney Margin (ODO4622-03) | 0.0 | 0.1 | Stomach Margin 9060394 | 4.3 | 0.0 |
| Kidney Cancer (ODO4450-01) | 0.0 | 0.0 | Gastric Cancer 9060397 | 0.0 | 0.0 |
| Kidney Margin (ODO4450-03) | 0.0 | 0.0 | Stomach Margin 9060396 | 0.0 | 0.0 |
| Kidney Cancer 8120607 | 0.0 | 0.0 | Gastric Cancer 064005 | 4.8 | 0.7 |

TABLE ID
Panel 4D
Column A - Rel. Exp. (%) Ag1201, Run 140237534
Column B - Rel. Exp. (%) Ag1201, Run 144180636

| Tissue Name | A | B | Tissue Name | A | B |
|---|---|---|---|---|---|
| Secondary Th1 act | 0.0 | 0.0 | HUVEC IL-1beta | 0.0 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + IFN gamma | 0.0 | 0.0 |
| Secondary Th1 rest | 0.0 | 0.0 | HUVEC TNF alpha +IL4 | 0.0 | 0.0 |
| Secondary Th2 rest | 0.0 | 0.0 | HUVEC IL-11 | 0.0 | 0.0 |
| Secondary Tr1 rest | 0.0 | 0.0 | Lung Microvascular EC none | 0.0 | 0.0 |
| Primary Th1 act | 0.0 | 0.0 | Lung Microvascular EC TNF alpha + | 0.0 | 0.0 |
| | | | IL-1beta | | |
| Primary Th2 act | 0.0 | 0.0 | Microvascular Dermal EC none | 0.0 | 0.0 |
| Primary Tr1 act | 0.0 | 0.0 | Microsvasular Dermal EC TNFalpha | 0.0 | 0.0 |
| | | | + IL-1beta | | |
| Primary Th1 rest | 0.0 | 0.0 | Bronchial epithelium TNFalpha + | 0.0 | 0.0 |
| | | | IL1beta | | |
| Primary Th2 rest | 0.0 | 0.0 | Small airway epithelium none | 0.0 | 0.0 |
| Primary Tr1 rest | 0.0 | 0.0 | Small airway epithelium TNFalpha + | 0.0 | 0.0 |
| | | | IL-1beta | | |
| CD45RA CD4 | 0.0 | 0.0 | Coronery artery SMG rest | 0.0 | 0.0 |
| lymphocyte act | | | | | |
| CD45RO CD4 | 0.0 | 0.0 | Coronery artery SMC TNFalpha + | 0.0 | 0.0 |
| lymphocyte act | | | IL-1beta | | |
| CD8 lymphocyte act | 2.3 | 0.0 | Astrocytes rest | 0.0 | 0.0 |
| Secondary CD8 | 0.0 | 0.0 | Astrocytes TNFalpha + IL-1beta | 4.3 | 0.0 |
| lymphocyte rest | | | | | |
| Secondary CD8 | 0.0 | 0.0 | KU-812 (Basophil) rest | 7.9 | 29.1 |
| lymphocyte act | | | | | |
| CD4 lymphocyte none | 0.0 | 0.0 | KU-812 (Basophil) PMA/ionomycin | 100.0 | 100.0 |
| 2ry Th1/Th2/Tr1 anti- | 0.0 | 0.0 | CCD1106 (Keratinocytes) none | 0.0 | 0.0 |
| CD95 CH11 | | | | | |
| LAK cells rest | 3.3 | 4.4 | 93580 CCD1106 (Keratinocytes) | 0.0 | 0.0 |
| | | | TNFa and IFNg | | |
| LAK cells IL-2 | 0.0 | 0.0 | Liver cirrhosis | 11.3 | 15.2 |
| LAK cells IL-2 + IL-12 | 0.0 | 0.0 | Lupus kidney | 0.0 | 0.0 |
| LAK cells IL-2 + IFN | 0.0 | 0.0 | NCI-H292 none | 0.0 | 0.0 |
| gamma | | | | | |
| LAK cells IL-2 + IL-18 | 0.0 | 0.0 | NCI-H292 IL-4 | 0.0 | 0.0 |
| LAK cells PMA/ | 0.0 | 0.0 | NCI-H292 IL-9 | 0.0 | 0.0 |
| ionomycin | | | | | |
| NK Cells IL-2 rest | 0.0 | 0.0 | NCI-H292 IL-13 | 0.0 | 0.0 |
| Two Way MLR 3 day | 3.4 | 0.0 | NCI-H292 IFN gamma | 0.0 | 0.0 |
| Two Way MLR 5 day | 8.3 | 0.0 | HPAEC none | 0.0 | 0.0 |
| Two Way MLR 7 day | 0.0 | 0.0 | HPAEC TNF alpha + IL-1 beta | 0.0 | 0.0 |
| PBMC rest | 0.0 | 0.0 | Lung fibroblast none | 0.0 | 0.0 |
| PBMC PWM | 0.0 | 0.0 | Lung fibroblast TNF alpha + IL-1 | 0.0 | 0.0 |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL-13 | 0.0 | 0.0 |
| ionomycin | | | | | |
| B lymphocytes PWM | 0.0 | 0.0 | Lung fibroblast IFN gamma | 0.0 | 0.0 |
| B lymphocytes CD40L | 0.0 | 0.0 | Dermal fibroblast CCD1070 rest | 0.0 | 0.0 |
| and IL-4 | | | | | |
| EOL-1 dbcAMP | 0.0 | 0.0 | Dermal fibroblast CCD1070 TNF | 0.0 | 0.0 |
| | | | alpha | | |
| EOL-1 dbcAMP | 0.0 | 0.0 | Dermal fibroblast CCD1070 IL-1 | 0.0 | 0.0 |
| PMA/ionomycin | | | beta | | |
| Dendritic cells none | 0.0 | 4.5 | Dermal fibroblast IFN gamma | 0.0 | 0.0 |
| Dendritic cells LPS | 0.0 | 0.0 | Dermal fibroblast IL-4 | 0.0 | 0.0 |
| Dendritic cells anti-CD40 | 7.9 | 4.7 | IBD Colitis 2 | 5.3 | 0.0 |
| Monocytes rest | 0.0 | 0.0 | IBD Crohn's | 0.0 | 0.0 |
| Monocytes LPS | 0.0 | 0.0 | Colon | 0.0 | 0.0 |
| Macrophages rest | 3.7 | 13.4 | Lung | 3.1 | 0.0 |
| Macrophages LPS | 0.0 | 4.2 | Thymus | 7.4 | 12.0 |
| HUVEC none | 0.0 | 0.0 | Kidney | 3.9 | 3.6 |
| HUVEC starved | 0.0 | 0.0 | | | |

Panel 1.3D Summary: Ag1201 Tissue expression of this was detected at a low level in many tissues. The highest expression was seen in testis. Expression of this gene or its protein product is useful as a marker for male germ cells and has therapeutic applications in fertility disorders as a potential target.

Panel 2D Summary: Ag1201 This gene was overexpressed in tumors derived from tissues responsive to steroid hormones—ovarian, uterine and prostate cancers as shown by panel 2D. It is therefore a marker for cells, especially tumor cells responsive to steroid hormones. Expression of this gene or its protein product are used to differentiate hormone-responsive and non-hormone responsive tumors, that are known to lead to different clinical outcomes. Being a GPCR, the protein is useful to screen candidate therapeutics for molecules able to modulate tumor growth, preferably small molecule therapeutic and human monoclonal antibodies.

Panel 4D Summary: Ag1201 The pattern of expression in panel 4 showed that Ag1201 has a potential role in inflammation, since this gene was expressed in activated basophils. Basophils are one of the key cell mediators of inflammation during asthma and allergy (Oliver J, Immunopharmacology 2000 Jul. 25; 48(3):269-81). This molecule is important in allowing these cells to extravasate into the site of inflammation and/or in the activation of these cells. Antibody therapeutics to Ag1201 are useful for the inhibition of nasal and lung inflammation due to basophil activation and effectively reduce or eliminate symptoms of asthma, emphysema, and allergic rhinitis.

J. CG50365-01: Carbonate Dehydratase

Expression of gene CG50365-01 was assessed using the primer-probe sets Ag2575 and Ag2644, described in Tables JA and JB. Results of the RTQ-PCR runs are shown in Tables JC, JD, JE and JF.

TABLE JA
Probe Name Ag2575

| | | | Start | SEQ ID |
|---|---|---|---|---|
| Primers | Sequences | Length | Position | No |
| Forward | 5′-tcagcaatctccaattgagatt-3′ | 22 | 96 | 1166 |
| Probe | TET-5′-tgaaatatgactcttccctccgacca-3′- | 26 | 131 | 1167 |
| | TAMRA | | | |
| Reverse | 5′-ttttagctgagcttgggtcata-3′ | 22 | 169 | 1168 |

TABLE JB
Probe Name Ag2644

| | | | Start | SEQ ID |
|---|---|---|---|---|
| Primers | Sequences | Length | Position | No |
| Forward | 5′-tcagcaatctccaattgagatt-3′ | 22 | 96 | 1169 |
| Probe | TET-5′-tgaaatatgactcttccctccgacca-3′- | 26 | 131 | 1170 |
| | TAMRA | | | |
| Reverse | 5′-ttttagctgagcttgggtcata-3′ | 22 | 169 | 1171 |

TABLE JC
Panel 1.3D
Column A - Rel. Exp.(%) Ag2575, Run 162430827
Column B - Rel. Exp.(%) Ag2575, Run 162431039

| Tissue Name | A | B | Tissue Name | A | B |
|---|---|---|---|---|---|
| Liver adenocarcinoma | 0.4 | 0.4 | Kidney (fetal) | 6.0 | 6.0 |
| Pancreas | 0.4 | 0.4 | Renal Ca. 786-0 | 13.5 | 13.5 |
| Pancreatic ca. CAPAN-2 | 4.0 | 4.0 | Renal Ca. A498 | 11.2 | 11.2 |
| Adrenal gland | 1.5 | 1.5 | Renal Ca. RXF 393 | 1.0 | 1.0 |
| Thyroid | 5.4 | 5.4 | Renal ca. ACHN | 2.9 | 2.9 |
| Salivaxy gland | 0.9 | 0.9 | Renal ca. UO-31 | 5.8 | 5.8 |
| Pituitary gland | 4.8 | 4.8 | Renal Ca. TK-10 | 6.9 | 6.9 |
| Brain (fetal) | 1.2 | 1.2 | Liver | 0.9 | 0.9 |
| Brain (whole) | 1.2 | 1.2 | Liver (fetal) | 3.7 | 3.7 |
| Brain (amygdala) | 3.1 | 3.1 | Liver ca. (hepatoblast) HepG2 | 10.0 | 10.0 |
| Brain (cerebellum) | 5.6 | 5.6 | Lung | 6.3 | 6.3 |
| Brain (hippocampus) | 5.4 | 5.4 | Lung (fetal) | 6.7 | 6.7 |
| Brain (substantia nigra) | 0.3 | 0.3 | Lung Ca. (small cell) LX-1 | 0.0 | 0.0 |
| Brain (thalamus) | 1.7 | 1.7 | Lung Ca. (small cell) NCI-H69 | 6.7 | 6.7 |
| Cerebral Cortex | 21.2 | 21.2 | Lung Ca. (s.cell var.) SHP-77 | 21.2 | 21.2 |
| Spinal cord | 2.8 | 2.8 | Lung Ca. (large cell) NCI-H460 | 4.4 | 4.4 |
| glio/astro U87-MG | 25.9 | 25.9 | Lung Ca. (non-sm. cell) A549 | 2.7 | 2.7 |
| glio/astro U-118-MG | 9.5 | 9.5 | Lung Ca. (non-s.cell) NCI-H23 | 3.0 | 3.0 |
| astrocytoma SW1783 | 17.4 | 17.4 | Lung Ca. (non-s.cell) HOP-62 | 4.8 | 4.8 |
| neuro*; met SK-N-AS | 0.0 | 0.0 | Lung Ca. (non-s.cl) NCI-H522 | 0.4 | 0.4 |
| astrocytoma SF-539 | 12.9 | 12.9 | Lung Ca. (squam.) SW 900 | 3.4 | 3.4 |
| astrocytoma SNB-75 | 2.8 | 2.8 | Lung Ca. (squam.) NCI-H596 | 2.7 | 2.7 |
| glioma SNB-19 | 27.0 | 27.0 | Mammary gland | 4.0 | 4.0 |
| glioma U251 | 4.6 | 4.6 | Breast ca.* (pl.ef) MCF-7 | 0.0 | 0.0 |
| glioma SF-295 | 5.2 | 5.2 | Breast ca.* (pl.ef) MDA-MB- | 7.5 | 7.5 |
| | | | 231 | | |
| Heart (Fetal) | 2.1 | 2.1 | Breast ca.* (pl.ef) T47D | 0.7 | 0.7 |
| Heart | 3.1 | 3.1 | Breast ca. BT-549 | 0.5 | 0.5 |
| Skeletal muscle (Fetal) | 3.6 | 3.6 | Breast Ca. MDA-N | 8.2 | 8.2 |
| Skeletal muscle | 0.0 | 0.0 | Ovary | 2.7 | 2.7 |
| Bone marrow | 0.8 | 0.8 | Ovarian Ca. OVCAR-3 | 5.9 | 5.9 |
| Thymus | 6.8 | 6.8 | Ovarian Ca. OVCAR-4 | 0.5 | 0.5 |
| Spleen | 1.6 | 1.6 | Ovarian Ca. OVCAR-5 | 35.1 | 35.1 |
| Lymph node | 1.4 | 1.4 | Ovarian Ca. OVCAR-8 | 12.8 | 12.8 |
| Colorectal | 5.7 | 5.7 | Ovarian Ca. IGROV-1 | 0.0 | 0.0 |
| Stomach | 2.6 | 2.6 | Ovarian Ca. (ascites) SK-OV-3 | 2.0 | 2.0 |
| Small intestine | 8.8 | 8.8 | Uterus | 1.4 | 1.4 |
| Colon Ca. SW480 | 0.0 | 0.0 | Placenta | 0.0 | 0.0 |
| Colon Ca.* SW620 (SW480 | 0.0 | 0.0 | Prostate | 0.8 | 0.8 |
| met) | | | | | |
| Colon Ca. HT29 | 8.4 | 8.4 | Prostate Ca.* (bone met) PC-3 | 7.0 | 7.0 |
| Colon Ca. HCT-116 | 5.7 | 5.7 | Testis | 2.2 | 2.2 |
| Colon Ca. CaCo-2 | 84.1 | 84.1 | Melanoma Hs688(A).T | 2.3 | 2.3 |
| CC Well to Mod Duff | 40.3 | 40.3 | Melanoma* (met) Hs688(B).T | 2.8 | 2.8 |
| (ODO3866) | | | | | |
| Colon Ca. HCC-2998 | 14.7 | 14.7 | Melanoma UACC-62 | 1.5 | 1.5 |
| Gastric ca. (liver met) NCI-N87 | 100.0 | 100.0 | Melanoma M14 | 4.3 | 4.3 |
| Bladder | 11.2 | 11.2 | Melanoma LOX IMVI | 7.6 | 7.6 |
| Trachea | 9.9 | 9.9 | Melanoma* (met) SK-MEL-5 | 13.0 | 13.0 |
| Kidney | 8.9 | 8.9 | Adipose | 11.0 | 11.0 |

TABLE JD
Panel 2D
Column A - Rel. Exp.(%) Ag2644, Run 162423326

| Tissue Name | A | Tissue Name | A |
|---|---|---|---|
| Normal Colon | 36.1 | Kidney Margin 8120608 | 2.9 |
| CC Well to Mod Diff (ODO3866) | 27.9 | Kidney Cancer 8120613 | 4.0 |
| CC Margin (ODO3866) | 11.1 | Kidney Margin 8120614 | 3.7 |
| CC Gr.2 rectosigmoid (ODO3868) | 19.1 | Kidney Cancer 9010320 | 10.2 |
| CC Margin (ODO3868) | 2.2 | Kidney Margin 9010321 | 8.0 |
| CC Mod Diff (ODO3920) | 21.0 | Normal Uterus | 0.0 |
| CC Margin (ODO3920) | 18.3 | Uterine Cancer 064011 | 12.2 |
| CC Gr.2 ascend colon (ODO3921) | 37.9 | Normal Thyroid | 12.8 |
| CC Margin (ODO3921) | 7.8 | Thyroid Cancer | 53.2 |
| CC from Partial Hepatectomy | 74.7 | Thyroid Cancer A302152 | 33.7 |
| (ODO4309) Mets | | | |
| Liver Margin (ODO4309) | 15.7 | Thyroid Margin A302153 | 13.6 |
| Colon mets to lung (OD04451-01) | 5.0 | Normal Breast | 27.7 |
| Lung Margin (OD04451-02) | 8.2 | Breast Cancer | 1.7 |
| Normal Prostate 6546-1 | 21.5 | Breast Cancer (OD04590-01) | 2.7 |
| Prostate Cancer (OD04410) | 10.7 | Breast Cancer Mets (0D04590-03) | 2.8 |
| Prostate Margin (OD04410) | 4.0 | Breast Cancer Metastasis | 35.1 |
| Prostate Cancer (OD04720-01) | 8.7 | Breast Cancer | 13.2 |
| Prostate Margin (OD04720-02) | 12.1 | Breast Cancer | 7.0 |
| Normal Lung | 24.8 | Breast Cancer 9100266 | 2.9 |
| Lung Met to Muscle (ODO4286) | 15.4 | Breast Margin 9100265 | 4.8 |
| Muscle Margin (ODO4286) | 1.5 | Breast Cancer A209073 | 18.6 |
| Lung Malignant Cancer (OD03126) | 7.9 | Breast Margin A209073 | 14.4 |
| Lung Margin (OD03126) | 22.4 | Normal Liver | 6.2 |
| Lung Cancer (OD04404) | 3.8 | Liver Cancer | 1.8 |
| Lung Margin (OD04404) | 10.6 | Liver Cancer 1025 | 3.4 |
| Lung Cancer (OD04565) | 2.2 | Liver Cancer 1026 | 3.6 |
| Lung Margin (OD04565) | 5.5 | Liver Cancer 6004-T | 4.8 |
| Lung Cancer (OD04237-01) | 14.7 | Liver Tissue 6004-N | 1.8 |
| Lung Margin (OD04237-02) | 18.0 | Liver Cancer 6005-T | 3.3 |
| Ocular Mel Met to Liver (ODO4310) | 1.0 | Liver Tissue 6005-N | 2.1 |
| Liver Margin (OD04310) | 5.4 | Normal Bladder | 10.7 |
| Melanoma Metastasis | 4.3 | Bladder Cancer | 1.3 |
| Lung Margin (OD04321) | 18.7 | Bladder Cancer | 4.6 |
| Normal Kidney | 35.4 | Bladder Cancer (OD04718-01) | 7.2 |
| Kidney Ca, Nuclear grade 2 (OD04338) | 26.6 | Bladder Normal Adjacent | 10.2 |
| | | (OD04718-03) | |
| Kidney Margin (OD04338) | 14.6 | Normal Ovary | 2.0 |
| Kidney Ca Nuclear grade 1/2 (OD04339) | 23.7 | Ovarian Cancer | 23.3 |
| Kidney Margin (OD04339) | 30.4 | Ovarian Cancer (OD04768-07) | 48.3 |
| Kidney Ca, Clear cell type (OD04340) | 19.3 | Ovary Margin (OD04768-08) | 2.7 |
| Kidney Margin (OD04340) | 22.7 | Normal Stomach | 21.0 |
| Kidney Ca, Nuclear grade 3 (OD04348) | 1.4 | Gastric Cancer 9060358 | 3.8 |
| Kidney Margin (OD04348) | 20.3 | Stomach Margin 9060359 | 15.8 |
| Kidney Cancer (OD04622-01) | 13.9 | Gastric Cancer 9060395 | 17.0 |
| Kidney Margin (OD04622-03) | 2.7 | Stomach Margin 9060394 | 15.8 |
| Kidney Cancer (OD04450-01) | 16.6 | Gastric Cancer 9060397 | 49.0 |
| Kidney Margin (OD04450-03) | 17.8 | Stomach Margin 9060396 | 12.2 |
| Kidney Cancer 8120607 | 2.9 | Gastric Cancer 064005 | 100.0 |

TABLE JE
Panel 3D
Column A - Rel. Exp.(%) Ag2644, Run 164886194

| Tissue Name | A | Tissue Name | A |
|---|---|---|---|
| 94905 Daoy | 5.5 | 94954 Ca Ski Cervical epidermoid | 16.5 |
| Medulloblastoma/Cerebellum | | carcinoma (metastasis | |
| 94906 TE671 | 0.0 | 94955 ES-2 Ovarian clear cell | 24.7 |
| Medulloblastom/Cerebellum | | carcinoma | |
| 94907 D283 Med | 15.3 | 94957 Ramos Stimulated with | 0.0 |
| Medulloblastoma/Cerebellum | | PMA/ionomycin 6 h | |
| 94908 PFSK-1 Primitive | 1.6 | 94958 Ramos Stimulated with | 2.2 |
| Neuroectodermal/Cerebellum | | PMA/ionomycin 14 h | |
| 94909 XF-498 CNS | 4.9 | 94962 MEG-01 Chronic | 30.8 |
| | | myelogenous leukemia | |
| | | (megokaryoblast) | |
| 94910 SNB-78 CNS/glioma | 8.8 | 94963 Raji Burkitt's lymphoma | 3.4 |
| 94911 SF-268 CNS/glioblastoma | 1.7 | 94964 Daudi Burkitt's lymphoma | 3.6 |
| 94912 T98G Glioblastoma | 0.0 | 94965 U266 B-cell | 5.9 |
| | | plasmacytoma/myeloma | |
| 96776 SK-N-SH Neuroblastoma | 0.0 | 94968 CA46 Burkitt's lymphoma | 3.2 |
| (metastasis) | | | |
| 94913 SF-295 CNS/glioblastoma | 10.3 | 94970 RL non-Hodgkin's B-cell | 0.0 |
| | | lymphoma | |
| 94914 Cerebellum | 8.1 | 94972 JM1 pre-B-cell | 0.0 |
| | | lymphoma/leukemia | |
| 96777 Cerebellum | 2.0 | 94973 Jurkat T cell leukemia | 3.3 |
| 94916 NCI-H292 Mucoepidermoid | 22.2 | 94974 TF-1 Erythroleukemia | 28.3 |
| lung carcinoma | | | |
| 94917 DMS-114 Small cell lung | 1.1 | 94975 HUT 78 T-cell lymphoma | 1.8 |
| cancer | | | |
| 94918 DMS-79 Small cell lung | 100.0 | 94977 U937 Histiocytic lymphoma | 4.4 |
| cancer/neuroendocrine | | | |
| 94919 NCI-H146 Small cell lung | 4.9 | 94980 KU-812 Myelogenous | 2.2 |
| cancer/neuroendocrine | | leukemia | |
| 94920 NCI-H526 Small cell lung | 6.9 | 769-P-Clear cell renal carcinoma | 3.8 |
| cancer/neuroendocrine | | | |
| 94921 NCI-N417 Small cell lung | 6.2 | 94983 Caki-2 Clear cell renal | 1.8 |
| cancer/neuroendocrine | | carcinoma | |
| 94923 NCI-H82 Small cell lung | 0.0 | 94984 SW 839 Clear cell renal | 4.1 |
| cancer/neuroendocrine | | carcinoma | |
| 94924 NCI-H157 Squamous cell lung | 30.6 | 94986 G401 Wilms' tumor | 0.0 |
| cancer (metastasis) | | | |
| 94925 NCI-H1155 Large cell lung | 14.9 | 94987 Hs766T Pancreatic carcinoma | 26.2 |
| cancer/neuroendocrine | | (LN metastasis) | |
| 94926 NCI-H1299 Large cell lung | 33.9 | 94988 CAPAN-1 Pancreatic | 14.4 |
| cancer/neuroendocrine | | adenocarcinoma (liver metastasis) | |
| 94927 NCI-H727 Lung carcinoid | 1.2 | 94989 SU86.86 Pancreatic carcinoma | 39.8 |
| | | (liver metastasis) | |
| 94928 NCI-UMC-11 Lung carcinoid | 4.1 | 94990 BxPC-3 Pancreatic | 3.3 |
| | | adenocarcinoma | |
| 94929 LX-1 Small cell lung cancer | 0.0 | 94991 HPAC Pancreatic | 1.4 |
| | | adenocarcinoma | |
| 94930 Colo-205 Colon cancer | 1.4 | 94992 MIA PaCa-2 Pancreatic | 2.1 |
| | | carcinoma | |
| 94931 KM12 Colon cancer | 31.9 | 94993 CFPAC-1 Pancreatic ductal | 32.8 |
| | | adenocarcinoma | |
| 94932 KM20L2 Colon cancer | 15.1 | 94994 PANC-1 Pancreatic epithelioid | 22.4 |
| | | ductal carcinoma | |
| 94933 NCI-H716 Colon cancer | 17.1 | 94996 T24 Bladder carcinma | 2.8 |
| | | (transitional cell | |
| 94935 SW-48 Colon adenocarcinoma | 28.5 | 5637-Bladder carcinoma | 11.4 |
| 94936 SW1116 Colon | 13.5 | 94998 HT-1197 Bladder carcinoma | 0.0 |
| adenocarcinoma | | | |
| 94937 LS 174T Colon | 56.3 | 94999 UM-UC-3 Bladder carcinma | 1.9 |
| adenocarcinoma | | (transitional cell) | |
| 94938 SW-948 Colon | 2.9 | 95000 A204 Rhabdomyosarcoma | 0.0 |
| adenocarcinoma | | | |
| 94939 SW-480 Colon | 10.9 | 95001 HT-1080 Fibrosarcoma | 4.5 |
| adenocarcinoma | | | |
| 94940 NCI-SNU-5 Gastric carcinoma | 0.0 | 95002 MG-63 Osteosarcoma (bone) | 2.2 |
| KATO III-Gastric carcinoma | 59.0 | 95003 SK-LMS-1 Leiomyosarcoma | 13.3 |
| | | (vulva) | |
| 94943 NCI-SNU-16 Gastric | 29.5 | 95004 SJRH30 Rhabdomyosarcoma | 5.6 |
| carcinoma | | (met to bone marrow) | |
| 94944 NCI-SNU-1 Gastric carcinoma | 15.8 | 95005 A431 Epidermoid carcinoma | 0.0 |
| 94946 RE-1 Gastric adenocarcinoma | 4.8 | 95007 WM266-4 Melanoma | 7.4 |
| 94947 RF-48 Gastric adenocarcinoma | 6.8 | DU 145-Prostate carcinoma (brain | 0.0 |
| | | metastasis) | |
| 96778 MKN-45 Gastric carcinoma | 1.8 | 95012 MDA-MB-468 Breast | 0.0 |
| | | adenocarcinoma | |
| 94949 NCI-N87 Gastric carcinoma | 24.7 | SCC-4-Squamous cell carcinoma of | 0.0 |
| | | tongue | |
| 94951 OVCAR-5 Ovarian carcinoma | 19.9 | SCC-9-Squamous cell carcinoma of | 0.0 |
| | | tongue | |
| 94952 RL95-2 Uterine carcinoma | 0.0 | SCC-15-Squamous cell carcinoma of | 0.0 |
| | | tongue | |
| 94953 HelaS3 Cervical | 0.0 | 95017 CAL 27 Squamous cell | 4.0 |
| adenocarcinoma | | carcinoma of tongue | |

TABLE JF
Panel 4D
Column A - Rel. Exp.(%) Ag2644, Run 158664089

| Tissue Name | A | Tissue Name | A |
|---|---|---|---|
| Secondary Th1 act | 0.5 | HUVEC IL-1beta | 3.3 |
| Secondary Th2 act | 0.4 | HUVEC IFN gamma | 14.4 |
| Secondary Tr1 act | 0.4 | HUVEC TNF alpha + IFN gamma | 27.7 |
| Secondary Th1 rest | 0.9 | HUVEC TNF alpha + IL4 | 12.2 |
| Secondary Th2 rest | 0.7 | HUVEC IL-11 | 1.0 |
| Secondary Tr1 rest | 0.9 | Lung Microvascular EC none | 1.1 |
| Primary Th1 act | 0.6 | Lung Microvascular EC TNF alpha + IL- | 6.2 |
| | | 1beta | |
| Primary Th2 act | 1.6 | Microvascular Dermal EC none | 1.9 |
| Primary Tr1 act | 1.4 | Microsvasular Dermal EC TNF alpha + IL- | 2.0 |
| | | 1beta | |
| Primary Th1 rest | 4.6 | Bronchial epithelium TNF alpha + IL1beta | 3.3 |
| Primary Th2 rest | 1.0 | Small airway epithelium none | 2.4 |
| Primary Tr1 rest | 1.8 | Small airway epithelium TNF alpha + IL- | 33.9 |
| | | 1beta | |
| CD45RA CD4 lymphocyte act | 1.9 | Coronery artery SMC rest | 6.1 |
| CD45RO CD4 lymphocyte act | 2.4 | Coronery artery SMC TNF alpha + IL-1beta | 2.4 |
| CD8 lymphocyte act | 1.0 | Astrocytes rest | 3.9 |
| Secondary CD8 lymphocyte rest | 1.4 | Astrocytes TNF alpha + IL-1beta | 2.1 |
| Secondary CD8 lymphocyte act | 0.8 | KU-812 (Basophil) rest | 1.3 |
| CD4 lymphocyte none | 0.8 | KU-812 (Basophil) PMA/ionomycin | 17.8 |
| 2ry Th1/Th2/Tr1 anti-CD95 | 0.6 | CCD1106 (Keratinocytes) none | 9.2 |
| CH11 | | | |
| LAK cells rest | 2.0 | CCD1106 (Keratinocytes) TNF alpha + IL- | 6.0 |
| | | 1beta | |
| LAK cells IL-2 | 5.4 | Liver cirrhosis | 2.1 |
| LAK cells IL-2 + IL-12 | 0.8 | Lupus kidney | 4.2 |
| LAK cells IL-2 + IFN gamma | 3.5 | NCI-H292 none | 35.8 |
| LAK cells IL-2 + IL-18 | 6.6 | NCI-H292 IL-4 | 52.9 |
| LAK cells PMA/ionomycin | 7.4 | NCI-H292 IL-9 | 45.1 |
| NK Cells IL-2 rest | 4.4 | NCI-H292 IL-13 | 25.7 |
| Two Way MLR 3 day | 3.4 | NCI-H292 IFN gamma | 42.9 |
| Two Way MLR 5 day | 1.4 | HPAEC none | 5.2 |
| Two Way MLR 7 day | 0.8 | HPAEC TNF alpha + IL-1beta | 16.3 |
| PBMC rest | 2.6 | Lung fibroblast none | 6.3 |
| PBMC PWM | 14.5 | Lung fibroblast TNF alpha + IL-1beta | 3.3 |
| PBMC PHA-L | 6.2 | Lung fibroblast IL-4 | 25.3 |
| Ramos (B cell) none | 4.9 | Lung fibroblast IL-9 | 7.6 |
| Ramos (B cell) ionomycin | 10.7 | Lung fibroblast IL-13 | 11.3 |
| B lymphocytes PWM | 9.1 | Lung fibroblast IFN gamma | 75.8 |
| B lymphocytes CD40L and IL-4 | 4.6 | Dermal fibroblast CCD1070 rest | 9.2 |
| EOL-1 dbcAMP | 0.0 | Dermal fibroblast CCD1070 TNF alpha | 11.0 |
| EOL-1 dbcAMP | 1.5 | Dermal fibroblast CCD1070 IL-1beta | 4.3 |
| PMA/ionomycin | | | |
| Dendritic cells none | 1.4 | Dermal fibroblast IFN gamma | 17.8 |
| Dendritic cells LPS | 1.1 | Dermal fibroblast IL-4 | 10.0 |
| Dendritic cells anti-CD40 | 0.9 | IBD Colitis 2 | 2.5 |
| Monocytes rest | 5.3 | IBD Crohn's | 15.8 |
| Monocytes LPS | 10.5 | Colon | 100.0 |
| Macrophages rest | 0.9 | Lung | 14.1 |
| Macrophages LPS | 1.1 | Thymus | 55.5 |
| HUVEC none | 7.3 | Kidney | 17.6 |
| HUVEC starved | 4.2 | | |

Panel 1.3D Summary: Ag2575 The expression of the CG50365-01 gene highest in a sample derived from a gastric cancer cell line (NCI-H87)(CTs=31). In addition, there was substantial expression in several colon cancer cell lines, ovarian cancer cell lines and brain cancer cell lines. Thus, the expression of this gene is useful as a marker to distinguish NCI-H87 cells from other samples in the panel. Therapeutic modulation of this gene, through the use of small molecule drugs, antibodies or protein therapeutics is of benefit in the treatment of colon cancer, brain cancer or ovarian cancer.

In addition, this gene was expressed at low levels in the cerebral cortex. Carbonate dehydratase plays an important role in modulating excitatory synaptic transmission in brain. (Parkkila S. Proc Natl Acad Sci USA 2001 Feb. 13; 98(4): 1918-23) Therefore, this molecule is of use in the treatment of schizophrenia, epilepsy, Alzheimer's disease, bipolar disorder, depression, or any clinical condition associated with impaired or altered neurotransmission.

Panel 2D Summary: Ag2644 The expression of the CG50365-01 gene was highest in a sample derived from a gastric cancer. In addition there was substantial expression associated with other gastric cancers, when compared to their adjacent normal tissues, as well as expression associated with ovarian cancer, breast cancer, thyroid cancer and colon cancer. This expression conformed with expression in Panel 1.3D. Expression of this is useful as a marker to distinguish this gastric cancer sample from other samples in the panel. Moreover, therapeutic modulation of this gene, through the use of small molecule drugs, antibodies or protein therapeutics is of benefit in the treatment of colon cancer, breast cancer, ovarian cancer, gastric cancer or thyroid cancer.

Panel 3D Summary: Ag2644 The expression of the CG50365-01 gene was highest in a sample derived from a lung cancer cell line (DMS-79). In addition there was expression associated with a colon cancer cell line, a gastric cancer cell line and a pancreatic cancer cell line. Thus, the expression of this gene is useful as a marker to distinguish DMS-79 cells from other samples in the panel. Moreover, therapeutic modulation of this gene, through the use of small molecule drugs, antibodies or protein therapeutics is of benefit in the treatment of colon cancer, pancreatic cancer, gastric cancer or lung cancer.

Panel 4D Summary: Ag2644 The CG50365-01 transcript was expressed in lung fibroblasts treated with gamma interferon, NCI-H292 cells regardless of treatment, activated basophil cell line, and gamma interferon treated HUVECs. It was also expressed in normal colon and thymus. The regulation of the transcript expression in fibroblasts and HUVECs showed that the protein encoded by this transcript contributes to the inflammatory changes due to gamma interferon. Therefore, therapies designed with the protein encoded by this transcript are important for the treatment of emphysema, psoriasis, arthritis and IBD.

K. CG50367-01: adam13

Expression of gene CG50367-01 was assessed using the primer-probe set Ag2425, described in Table KA. Results of the RTQ-PCR runs are shown in Tables KB, KC, KD and KE.

TABLE KA
Probe Name Ag2425

| | | | Start | SEQ ID |
|---|---|---|---|---|
| Primers | Sequences | Length | Position | No |
| Forward | 5′-ggctcctgctgaccatattc-3′ | 20 | 2342 | 1172 |
| Probe | TET-5′-catttaccctccaccatttctcccag-3′- | 26 | 2366 | 1173 |
| | TAMRA | | | |
| Reverse | 5′-gctgggctcatgagagttct-3′ | 20 | 2398 | 1174 |

TABLE KB
Ardais Prostate 1.0
Column A - Rel. Exp.(%) Ag2425, Run 321632641

| Tissue Name | A | Tissue Name | A |
|---|---|---|---|
| 151135 Prostate NAT(B87) | 22.8 | 151128 Prostate cancer(B8C) | 8.2 |
| 151143 Prostate NAT(B8A) | 28.5 | 151136 Prostate cancer(B8B) | 1.3 |
| 153669 Prostate NAT(D5E) | 18.8 | 151144 Prostate cancer(B8F) | 8.1 |
| 153677 Prostate NAT(D66) | 8.1 | 153654 Prostate cancer(D4F) | 3.5 |
| 153685 Prostate NAT(D6E) | 14.0 | 153662 Prostate cancer(D57) | 0.9 |
| 145905 Prostate NAT(A0C) | 14.8 | 153655 Prostate cancer(D50) | 27.2 |
| 153670 Prostate NAT(D5F) | 3.3 | 145907 Prostate cancer(A0A) | 13.0 |
| 153678 Prostate NAT(D67) | 12.2 | 153663 Prostate cancer(D58) | 100.0 |
| 153686 Prostate NAT(D6F) | 1.8 | 151130 Prostate cancer(B90) | 9.1 |
| 145906 Prostate NAT(A09) | 9.1 | 153648 Prostate cancer(D49) | 9.9 |
| 151129 Prostate NAT(B93) | 60.3 | 153656 Prostate cancer(D51) | 9.8 |
| 151137 Prostate NAT(B86) | 18.7 | 153664 Prostate cancer(D59) | 1.8 |
| 153671 Prostate NAT(D60) | 4.4 | 155799 Prostate cancer(EA8) | 6.2 |
| 151145 Prostate NAT(B91) | 26.8 | 145909 Prostate cancer(9E7) | 4.9 |
| 153679 Prostate NAT(D68) | 19.2 | 153649 Prostate cancer(D4A) | 2.4 |
| 153687 Prostate NAT(D70) | 10.2 | 153657 Prostate cancer(D52) | 1.3 |
| 153672 Prostate NAT(D61) | 8.1 | 153665 Prostate cancer(D5A) | 3.9 |
| 153680 Prostate NAT(D69) | 23.7 | 151132 Prostate cancer(B88) | 3.2 |
| 151131 Prostate NAT(B85) | 5.2 | 153650 Prostate cancer(D4B) | 1.8 |
| 153673 Prostate NAT(D62) | 8.2 | 153658 Prostate cancer(D53) | 1.8 |
| 153681 Prostate | 7.4 | 153666 Prostate cancer(D5B) | 0.4 |
| NAT(D6A) | | | |
| 145910 Prostate NAT(9C3) | 18.8 | 153651 Prostate cancer(D4C) | 2.4 |
| 153674 Prostate NAT(D63) | 24.0 | 153659 Prostate cancer(D54) | 7.2 |
| 153682 Prostate NAT(D6B) | 6.4 | 153667 Prostate cancer(D5C) | 7.6 |
| 151133 Prostate NAT(B94) | 7.6 | 151134 Prostate cancer(B92) | 1.4 |
| 153675 Prostate NAT(D64) | 3.6 | 151142 Prostate cancer(B89) | 5.5 |
| 153683 Prostate NAT(D6C) | 5.1 | 153652 Prostate cancer(D4D) | 7.1 |
| 153668 Prostate | 4.7 | 153660 Prostate cancer(D55) | 0.9 |
| NAT(D5D) | | | |
| 153676 Prostate NAT(D65) | 1.2 | 149773 Prostate NAT(AD8) | 0.0 |
| 153684 Prostate | 6.9 | 149774 Prostate cancer(AD7) | 6.5 |
| NAT(D6D) | | | |
| 145904 Prostate | 21.2 | 151139 Prostate NAT(B8E) | 15.1 |
| cancer(9E2) | | | |
| 149776 Prostate | 4.5 | 151138 Prostate cancer(B8D) | 2.2 |
| cancer(AD5) | | | |
| 153653 Prostate | 2.7 | 151141 Prostate NAT(B96) | 6.8 |
| cancer(D4E) | | | |
| 153661 Prostate | 6.2 | 151140 Prostate cancer(B95) | 4.7 |
| cancer(D56) | | | |

TABLE KC
Panel 1.3D
Column A - Rel. Exp.(%) Ag2425, Run 155561580

| Tissue Name | A | Tissue Name | A |
|---|---|---|---|
| Liver adenocarcinoma | 0.0 | Kidney (fetal) | 3.9 |
| Pancreas | 1.8 | Renal ca. 786-0 | 0.0 |
| Pancreatic ca. | 0.0 | Renal ca. A498 | 0.0 |
| CAPAN 2 | | | |
| Adrenal gland | 0.9 | Renal ca. RXF 393 | 0.0 |
| Thyroid | 2.7 | Renal ca. ACHN | 1.6 |
| Salivary gland | 1.1 | Renal ca. UO-31 | 0.0 |
| Pituitary gland | 0.5 | Renal ca. TK-10 | 0.0 |
| Brain (fetal) | 4.6 | Liver | 0.0 |
| Brain (whole) | 2.3 | Liver (fetal) | 1.2 |
| Brain (amygdala) | 4.2 | Liver ca. (hepatoblast) HepG2 | 0.0 |
| Brain (cerebellum) | 0.0 | Lung | 2.8 |
| Brain (hippocampus) | 25.3 | Lung (fetal) | 17.9 |
| Brain | 2.4 | Lung ca. (small cell) LX-1 | 0.0 |
| (substantia nigra) | | | |
| Brain (thalamus) | 9.4 | Lung ca. (small cell) NCI-H69 | 0.0 |
| Cerebral Cortex | 1.5 | Lung ca. (s. cell var.) SHP-77 | 1.0 |
| Spinal cord | 3.9 | Lung ca. (large cell)NCI-H460 | 0.0 |
| glio/astro U87-MG | 0.0 | Lung ca. (non-sm. cell) A549 | 1.7 |
| glio/astro U-118-MG | 1.1 | Lung ca. (non-s. cell) NCI-H23 | 1.8 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.8 | Mammary gland | 13.5 |
| glioma U251 | 0.0 | Breast ca.* (pl.ef) MCF-7 | 0.0 |
| glioma SF-295 | 0.0 | Breast ca.* (pl.ef) MDA-MB-231 | 0.0 |
| Heart (Fetal) | 3.4 | Breast ca.* (pl.ef) T47D | 0.0 |
| Heart | 1.1 | Breast ca. BT-549 | 1.6 |
| Skeletal muscle (Fetal) | 100.0 | Breast ca. MDA-N | 0.0 |
| Skeletal muscle | 0.9 | Ovary | 1.9 |
| Bone marrow | 3.3 | Ovarian ca. OVCAR-3 | 0.0 |
| Thymus | 4.1 | Ovarian ca. OVCAR-4 | 0.0 |
| Spleen | 2.7 | Ovarian ca. OVCAR-5 | 0.0 |
| Lymph node | 4.6 | Ovarian ca. OVCAR-8 | 0.0 |
| Colorectal | 5.9 | Ovarian ca. IGROV-1 | 0.0 |
| Stomach | 7.3 | Ovarian ca. (ascites) SK-OV-3 | 0.0 |
| Small intestine | 18.4 | Uterus | 37.4 |
| Colon ca. SW480 | 0.0 | Placenta | 1.8 |
| Colon ca.* SW620 | 0.0 | Prostate | 8.8 |
| (SW480 met) | | | |
| Colon ca. HT29 | 0.0 | Prostate ca.* (bone met) PC-3 | 0.0 |
| Colon ca. HCT-116 | 0.0 | Testis | 7.5 |
| Colon ca. CaCo-2 | 0.0 | Melanoma Hs688(A).T | 5.0 |
| CC Well to Mod Diff | 0.0 | Melanoma* (met) Hs688(B).T | 3.3 |
| (ODO3866) | | | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 15.8 | Melanoma* (met) SK-MEL-5 | 0.0 |
| Kidney | 1.8 | Adipose | 1.6 |

TABLE KD
Panel 2D
Column A - Rel. Exp.(%) Ag2425, Run 155562155

| Tissue Name | A | Tissue Name | A |
|---|---|---|---|
| Normal Colon | 100.0 | Kidney Margin 8120608 | 0.0 |
| CC Well to Mod Diff (ODO3866) | 5.6 | Kidney Cancer 8120613 | 0.0 |
| CC Margin (ODO3866) | 4.5 | Kidney Margin 8120614 | 2.1 |
| CC Gr.2 rectosigmoid (ODO3868) | 20.7 | Kidney Cancer 9010320 | 2.2 |
| CC Margin (ODO3868) | 21.9 | Kidney Margin 9010321 | 6.1 |
| CC Mod Diff (ODO3920) | 6.7 | Normal Uterus | 49.3 |
| CC Margin (ODO3920) | 61.6 | Uterine Cancer 064011 | 92.7 |
| CC Gr.2 ascend colon (ODO3921) | 1.0 | Normal Thyroid | 18.3 |
| CC Margin (ODO3921) | 6.2 | Thyroid Cancer | 0.0 |
| CC from Partial Hepatectomy | 0.0 | Thyroid Cancer A302152 | 2.8 |
| (ODO4309) Mets | | | |
| Liver Margin (ODO4309) | 0.0 | Thyroid Margin A302153 | 20.4 |
| Colon mets to lung (OD04451-01) | 0.0 | Normal Breast | 53.2 |
| Lung Margin (OD04451-02) | 0.0 | Breast Cancer | 0.0 |
| Normal Prostate 6546-1 | 66.4 | Breast Cancer (OD04590-01) | 2.6 |
| Prostate Cancer (OD04410) | 25.9 | Breast Cancer Mets (OD04590-03) | 11.5 |
| Prostate Margin (OD04410) | 72.7 | Breast Cancer Metastasis | 0.0 |
| Prostate Cancer (OD04720-01) | 45.4 | Breast Cancer | 24.7 |
| Prostate Margin (OD04720-02) | 33.7 | Breast Cancer | 51.8 |
| Normal Lung | 84.1 | Breast Cancer 9100266 | 3.1 |
| Lung Met to Muscle (ODO4286) | 9.4 | Breast Margin 9100265 | 12.9 |
| Muscle Margin (ODO4286) | 0.0 | Breast Cancer A209073 | 17.3 |
| Lung Malignant Cancer (OD03126) | 0.0 | Breast Margin A209073 | 99.3 |
| Lung Margin (OD03126) | 10.6 | Normal Liver | 0.0 |
| Lung Cancer (OD04404) | 0.0 | Liver Cancer | 4.0 |
| Lung Margin (OD04404) | 0.0 | Liver Cancer 1025 | 4.9 |
| Lung Cancer (OD04565) | 13.7 | Liver Cancer 1026 | 0.0 |
| Lung Margin (OD04565) | 10.6 | Liver Cancer 6004-T | 0.0 |
| Lung Cancer (OD04237-01) | 0.0 | Liver Tissue 6004-N | 8.5 |
| Lung Margin (OD04237-02) | 0.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 11.1 |
| Melanoma Metastasis | 0.0 | Bladder Cancer | 3.1 |
| Lung Margin (OD04321) | 2.9 | Bladder Cancer | 14.6 |
| Normal Kidney | 9.7 | Bladder Cancer (OD04718-01) | 2.4 |
| Kidney Ca, Nuclear grade 2 (OD04338) | 0.0 | Bladder Normal Adjacent | 10.6 |
| | | (OD04718-03) | |
| Kidney Margin (OD04338) | 6.0 | Normal Ovary | 0.0 |
| Kidney Ca Nuclear grade ½ (OD04339) | 0.0 | Ovarian Cancer | 0.0 |
| Kidney Margin (OD04339) | 0.0 | Ovarian Cancer (OD04768-07) | 3.2 |
| Kidney Ca, Clear cell type (OD04340) | 0.0 | Ovary Margin (OD04768-08) | 6.0 |
| Kidney Margin (OD04340) | 4.0 | Normal Stomach | 12.7 |
| Kidney Ca, Nuclear grade 3 (OD04348) | 0.0 | Gastric Cancer 9060358 | 9.9 |
| Kidney Margin (OD04348) | 6.7 | Stomach Margin 9060359 | 0.0 |
| Kidney Cancer (OD04622-01) | 0.0 | Gastric Cancer 9060395 | 39.2 |
| Kidney Margin (OD04622-03) | 7.4 | Stomach Margin 9060394 | 26.4 |
| Kidney Cancer (OD04450-01) | 0.0 | Gastric Cancer 9060397 | 6.2 |
| Kidney Margin (OD04450-03) | 0.0 | Stomach Margin 9060396 | 3.3 |
| Kidney Cancer 8120607 | 0.0 | Gastric Cancer 064005 | 25.3 |

TABLE KE
Panel 4D
Column A - Rel. Exp.(%) Ag2425, Run 155562267

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 0.0 | Microvascular Dermal EC none | 0.0 |
| Primary Tr1 act | 1.3 | Microsvasular Dermal EC TNF alpha + IL- | 0.0 |
| | | 1beta | |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL- | 0.0 |
| | | 1beta | |
| CD45RA CD4 lymphocyte act | 1.8 | Coronery artery SMC rest | 0.0 |
| CD45RO CD4 lymphocyte act | 1.4 | Coronery artery SMC TNF alpha + IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 1.3 | KU-812 (Basophil) PMA/ionomycin | 0.0 |
| 2ry Th1/Th2/Tr1 anti-CD95 | 3.9 | CCD1106 (Keratinocytes) none | 0.0 |
| CH11 | | | |
| LAK cells rest | 0.0 | CCD1106 (Keratinocytes) TNF alpha + IL- | 0.0 |
| | | 1beta | |
| LAK cells IL-2 | 0.0 | Liver cirrhosis | 4.9 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 13.0 |
| PBMC PWM | 0.0 | Lung fibroblast TNF alpha + IL-1beta | 2.7 |
| PBMC PHA-L | 0.0 | Lung fibroblast IL-4 | 3.4 |
| Ramos (B cell) none | 0.0 | Lung fibroblast IL-9 | 10.7 |
| Ramos (B cell) ionomycin | 0.0 | Lung fibroblast IL-13 | 5.9 |
| B lymphocytes PWM | 0.0 | Lung fibroblast IFN gamma | 3.3 |
| B lymphocytes CD40L and IL-4 | 0.0 | Dermal fibroblast CCD1070 rest | 23.7 |
| EOL-1 dbcAMP | 0.0 | Dermal fibroblast CCD1070 TNF alpha | 5.6 |
| EOL-1 dbcAMP | 0.0 | Dermal fibroblast CCD1070 IL-1beta | 12.5 |
| PMA/ionomycin | | | |
| Dendritic cells none | 0.0 | Dermal fibroblast IFN gamma | 64.6 |
| Dendritic cells LPS | 0.0 | Dermal fibroblast IL-4 | 100.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 28.3 |
| Macrophages rest | 0.0 | Lung | 21.6 |
| Macrophages LPS | 0.0 | Thymus | 0.0 |
| HUVEC none | 0.0 | Kidney | 0.0 |
| HUVEC starved | 0.0 | | |

Ardais Prostate 1.0 Summary: Ag2425 Expression of the CG50367-01 gene was highest in a prostate cancer sample (CT=28.3), with expression of this gene slightly downregulated in most of the prostate cancer samples.

Panel 1.3D Summary: Ag2425 Highest expression of the CG50367-01 gene was seen in fetal skeletal muscle (CT=31.1). This gene was highly expressed in fetal skeletal muscle when compared to expression in adult skeletal muscle (CT=40). Thus expression of this gene is useful as a marker differentiate between fetal and adult skeletal muscle. Furthermore, the higher levels of expression in the fetal source of the tissue show that the protein encoded by this gene is involved in the development of the skeletal muscle in the fetus. Therapeutic modulation of the expression or function of this gene is useful for restoring muscle mass or function to weak or dystrophic muscle in the adult.

This gene was expressed at a very low level in all the cancer cell lines used in this panel. The absence of exprssion of this gene in the cancer cell lines showed that modulation of the function of the gene product through the use of peptides, polypeptides, chimeric molecules or small molecule drugs, aree useful in the therapy of cancer.

This gene is a cell-surface metalloprotease expressed at low levels in the hippocampus. It is useful in the treatment of diseases in which the hippocampus is involved, such as Alzheimer's disease, Parkinson's disease, schizophrenia, bipolar disorder, or temporal lobe epilepsy.

Panel 2D Summary: Ag2425 The CG50367-01 gene was expressed at low levels in this panel, with highest expression in the colon (CT=32.2). Moderately higher levels of expression were seen in normal breast, uterine and thyroid tissues compared to the adjacent cancers. Expression of this gene is useful as a marker to identify normal tissue from cancerous tissue in these organs. Therapeutic modulation of the activity of the product of this gene, through the use of peptides, polypeptides, chimeric molecules or small molecule drugs, is useful in the therapy of these cancers.

Panel 4D Summary: Ag2425 The CG50367-01 transcript was most highly expressed in dermal fibroblast upon treatment with either Il-4 or IFN gamma (CTs=31-32) and at lower levels in resting dermal fibroblasts. This transcript was also expressed in lung fibroblasts and normal lung and thymus. This transcript encodes for a ADAM like protein, a member of membrane-anchored glycoproteins that play a role in diverse cellular processes from cell-cell interaction to shedding of cell surface proteases. The expression of this transcript in dermal and lung fibroblasts showed that the protein encoded by this transcript is involved in diseases associated with fibrosis or fibroplasia. Modulation of the expression or the function of this molecule is useful for the treatment of psoriasis, chronic obstructive pulmonary diseases and potentially for osteoarthritis and rheumatoid arthritis.

L. CG50718-02 and CG50718-06: Glomerular Mesangial Cell Receptor Protein-Tyrosine Phosphatase Precursor,

Expression of gene CG50718-02 and variant CG50718-06 was assessed using the primer-probe sets Ag1555 and Ag2315, described in Tables LA and LB. Results of the RTQ-PCR runs are shown in Tables LC, LD, LE, and LF.

TABLE LA
Probe Name Ag1555

| | | | Start | SEQ ID |
|---|---|---|---|---|
| Primers | Sequences | Length | Position | No |
| Forward | 5′-gaagtgaaagaatgtgcatggt-3′ | 22 | 6707 | 1175 |
| Probe | TET-5′-caccagtgcattctggatctcttatca- | 27 | 6757 | 1176 |
| | 3′-TAMRA | | | |
| Reverse | 5′-tgggctgattacttcccttatt-3′ | 22 | 6784 | 1177 |

TABLE LB
Probe Name Ag2315

| | | | Start | SEQ ID |
|---|---|---|---|---|
| Primers | Sequences | Length | Position | No |
| Forward | 5′-agatgagtcagtgccgttagc-3′ | 21 | 3711 | 1178 |
| Probe | TET-5′-cctccacaaaatttgactttaatcaactg- | 29 | 3733 1179 | |
| | 3′-TAMRA | | | |
| Reverse | 5′-tccatttcagccatacaaagtc-3′ | 22 | 3769 | 1180 |

TABLE LC
Panel 1.3D
Column A - Rel. Exp.(%) Ag1555, Run 146380268
Column B - Rel. Exp.(%) Ag1555, Run 147775028

| Tissue Name | A | B | Tissue Name | A | B |
|---|---|---|---|---|---|
| Liver adenocarcinoma | 0.0 | 0.0 | Kidney (fetal) | 33.9 | 37.6 |
| Pancreas | 5.8 | 1.6 | Renal Ca. 786-0 | 0.0 | 0.0 |
| Pancreatic ca. CAPAN 2 | 0.0 | 0.0 | Renal Ca. A498 | 0.0 | 0.0 |
| Adrenal gland | 0.0 | 1.9 | Renal Ca. RXF 393 | 0.0 | 0.0 |
| Thyroid | 8.3 | 24.7 | Renal ca. ACHN | 0.0 | 0.0 |
| Salivary gland | 1.0 | 0.0 | Renal ca. UO-31 | 0.0 | 1.4 |
| Pituitary gland | 0.0 | 0.0 | Renal Ca. TK-10 | 0.0 | 0.0 |
| Brain (fetal) | 0.6 | 0.0 | Liver | 0.0 | 0.0 |
| Brain (whole) | 1.3 | 1.6 | Liver (fetal) | 0.0 | 3.6 |
| Brain (amygdala) | 3.4 | 4.0 | Liver ca. (hepatoblast) HepG2 | 0.0 | 0.0 |
| Brain (cerebellum) | 0.0 | 0.0 | Lung | 51.1 | 52.5 |
| Brain (hippocampus) | 1.2 | 0.6 | Lung (fetal) | 100.0 | 100.0 |
| Brain (substantia nigra) | 0.0 | 0.0 | Lung Ca. (small cell) LX-1 | 0.0 | 0.0 |
| Brain (thalamus) | 3.2 | 1.3 | Lung Ca. (small cell) NCI-H69 | 2.4 | 0.0 |
| Cerebral Cortex | 0.0 | 0.0 | Lung Ca. (s.cell var.) SHP-77 | 0.0 | 0.0 |
| Spinal cord | 1.1 | 0.0 | Lung Ca. (large cell) NCI-H460 | 0.0 | 0.0 |
| glio/astro U87-MG | 0.0 | 2.7 | Lung Ca. (non-sm. cell) A549 | 0.0 | 0.0 |
| glio/astro U-118-MG | 27.2 | 34.6 | Lung Ca. (non-s.cell) NCI-H23 | 0.0 | 0.0 |
| astrocytoma SW1783 | 5.4 | 13.8 | Lung Ca. (non-s.cell) HOP-62 | 0.7 | 0.9 |
| neuro*; met SK-N-AS | 0.0 | 0.6 | Lung Ca. (non-s.cl) NCI-H522 | 9.9 | 5.4 |
| astrocytoma SF-539 | 0.8 | 0.0 | Lung Ca. (squam.) SW 900 | 0.0 | 0.0 |
| astrocytoma SNB-75 | 0.0 | 0.0 | Lung Ca. (squam.) NCI-H596 | 1.3 | 2.2 |
| glioma SNB-19 | 0.0 | 0.0 | Mammary gland | 13.0 | 26.6 |
| glioma U251 | 0.0 | 0.0 | Breast ca.* (pl.ef) MCF-7 | 3.9 | 0.9 |
| glioma SF-295 | 1.3 | 3.3 | Breast ca.* (pl.ef) MDA-MB- | 0.0 | 0.0 |
| | | | 231 | | |
| Heart (Fetal) | 0.0 | 0.0 | Breast ca.* (pl.ef) T47D | 0.0 | 0.0 |
| Heart | 0.0 | 5.7 | Breast ca. BT-549 | 0.0 | 0.0 |
| Skeletal muscle (Fetal) | 3.5 | 1.6 | Breast Ca. MDA-N | 0.0 | 0.0 |
| Skeletal muscle | 0.0 | 1.4 | Ovary | 5.2 | 1.6 |
| Bone marrow | 1.0 | 4.1 | Ovarian Ca. OVCAR-3 | 0.0 | 0.0 |
| Thymus | 1.0 | 0.0 | Ovarian Ca. OVCAR-4 | 0.0 | 0.0 |
| Spleen | 0.0 | 0.0 | Ovarian Ca. OVCAR-5 | 0.0 | 0.0 |
| Lymph node | 3.7 | 4.8 | Ovarian Ca. OVCAR-8 | 0.0 | 0.0 |
| Colorectal | 0.0 | 0.0 | Ovarian Ca. IGROV-1 | 0.0 | 0.0 |
| Stomach | 1.2 | 2.3 | Ovarian Ca. (ascites) SK-OV-3 | 0.0 | 0.0 |
| Small intestine | 2.2 | 6.7 | Uterus | 0.0 | 0.9 |
| Colon Ca. SW480 | 0.0 | 0.0 | Placenta | 11.8 | 27.7 |
| Colon Ca.* SW620 (SW480 | 0.0 | 0.0 | Prostate | 3.5 | 0.9 |
| met) | | | | | |
| Colon Ca. HT29 | 0.0 | 0.0 | Prostate Ca.* (bone met) PC-3 | 0.0 | 0.0 |
| Colon Ca. HCT-116 | 0.0 | 0.0 | Testis | 58.2 | 67.4 |
| Colon Ca. CaCo-2 | 0.0 | 0.0 | Melanoma Hs688(A).T | 22.7 | 52.1 |
| CC Well to Mod Duff | 0.0 | 0.0 | Melanoma* (met) Hs688(B).T | 4.8 | 4.2 |
| (ODO3866) | | | | | |
| Colon Ca. HCC-2998 | 0.0 | 0.0 | Melanoma UACC-62 | 0.0 | 1.5 |
| Gastric ca. (liver met) NCI-N87 | 0.0 | 0.0 | Melanoma M14 | 0.0 | 0.0 |
| Bladder | 2.0 | 0.0 | Melanoma LOX IMVI | 0.0 | 0.0 |
| Trachea | 2.4 | 3.6 | Melanoma* (met) SK-MEL-5 | 0.0 | 0.0 |
| Kidney | 15.5 | 17.8 | Adipose | 38.2 | 40.6 |

TABLE LD
Panel 2D
Column A - Rel. Exp.(%) Ag1555, Run 147775063
Column B - Rel. Exp.(%) Ag1555, Run 159601974
Column C - Rel. Exp.(%) Ag2315, Run 159200827

| Tissue Name | A | B | C | Tissue Name | A | B | C |
|---|---|---|---|---|---|---|---|
| Normal Colon | 3.8 | 7.1 | 12.4 | Kidney Margin 8120608 | 2.9 | 1.2 | 1.7 |
| CC Well to Mod Diff | 1.0 | 0.0 | 2.3 | Kidney Cancer 8120613 | 0.0 | 0.0 | 0.0 |
| (ODO3866) | | | | | | | |
| CC Margin (ODO3866) | 0.0 | 0.0 | 0.7 | Kidney Margin 8120614 | 1.2 | 2.6 | 1.8 |
| CC Gr.2 rectosigmoid | 0.0 | 0.0 | 0.0 | Kidney Cancer 9010320 | 2.7 | 2.6 | 2.1 |
| (ODO3868) | | | | | | | |
| CC Margin (ODO3868) | 0.0 | 0.7 | 0.0 | Kidney Margin 9010321 | 6.6 | 5.9 | 4.9 |
| CC Mod Diff (ODO3920) | 0.0 | 0.0 | 0.0 | Normal Uterus | 0.0 | 0.0 | 1.8 |
| CC Margin (ODO3920) | 0.0 | 0.0 | 2.2 | Uterine Cancer 064011 | 0.0 | 0.0 | 4.5 |
| CC Gr.2 ascend colon | 0.0 | 0.0 | 0.0 | Normal Thyroid | 34.9 | 27.4 | 11.4 |
| (ODO3921) | | | | | | | |
| CC Margin (ODO3921) | 0.0 | 0.0 | 0.0 | Thyroid Cancer | 2.9 | 7.2 | 7.9 |
| CC from Partial | 1.6 | 1.1 | 0.0 | Thyroid Cancer | 1.3 | 3.3 | 2.0 |
| Hepatectomy (ODO4309) | | | | A302152 | | | |
| Mets | | | | | | | |
| Liver Margin (ODO4309) | 0.0 | 0.0 | 2.0 | Thyroid Margin | 49.7 | 69.7 | 72.2 |
| | | | | A302153 | | | |
| Colon mets to lung | 2.0 | 1.0 | 0.5 | Normal Breast | 10.0 | 8.9 | 25.3 |
| (ODO4451-01) | | | | | | | |
| Lung Margin (ODO4451-02) | 8.6 | 10.0 | 10.9 | Breast Cancer | 10.2 | 3.0 | 1.1 |
| Normal Prostate 6546-1 | 4.2 | 12.2 | 1.4 | Breast Cancer | 0.0 | 2.8 | 3.9 |
| | | | | (ODO4590-01) | | | |
| Prostate Cancer (ODO4410) | 0.0 | 0.0 | 3.4 | Breast Cancer Mets | 7.8 | 7.3 | 7.9 |
| | | | | (ODO4590-03) | | | |
| Prostate Margin (ODO4410) | 0.8 | 6.4 | 2.2 | Breast Cancer | 4.1 | 8.0 | 3.5 |
| | | | | Metastasis | | | |
| Prostate Cancer (ODO4720- | 9.5 | 11.7 | 19.6 | Breast Cancer | 0.0 | 0.0 | 1.2 |
| 01) | | | | | | | |
| Prostate Margin (ODO4720- | 10.0 | 11.3 | 24.5 | Breast Cancer | 3.7 | 2.9 | 0.9 |
| 02) | | | | | | | |
| Normal Lung | 59.9 | 61.1 | 87.7 | Breast Cancer 9100266 | 2.2 | 1.1 | 1.5 |
| Lung Met to Muscle | 0.0 | 0.0 | 0.0 | Breast Margin 9100265 | 0.0 | 0.0 | 0.5 |
| (ODO4286) | | | | | | | |
| Muscle Margin (ODO4286) | 0.9 | 0.0 | 1.8 | Breast Cancer A209073 | 0.7 | 1.1 | 1.9 |
| Lung Malignant Cancer | 1.9 | 2.8 | 1.7 | Breast Margin A209073 | 0.0 | 1.2 | 0.9 |
| (ODO3126) | | | | | | | |
| Lung Margin (ODO3126) | 36.3 | 3.6 | 43.8 | Normal Liver | 0.0 | 0.0 | 0.0 |
| Lung Cancer (ODO4404) | 2.2 | 4.4 | 4.3 | Liver Cancer | 0.0 | 0.0 | 0.6 |
| Lung Margin (ODO4404) | 9.5 | 4.2 | 8.4 | Liver Cancer 1025 | 0.0 | 0.0 | 0.0 |
| Lung Cancer (ODO4565) | 0.0 | 0.0 | 00 | Liver Cancer 1026 | 0.0 | 0.0 | 0.0 |
| Lung Margin (ODO4565) | 10.8 | 9.7 | 14.1 | Liver Cancer 6004-T | 0.0 | 1.0 | 0.6 |
| Lung Cancer (OD04237-01) | 0.0 | 0.0 | 0.0 | Liver Tissue 6004-N | 0.0 | 0.0 | 0.0 |
| Lung Margin (ODO4237-02) | 30.1 | 18.4 | 29.3 | Liver Cancer 6005-T | 0.0 | 0.0 | 0.0 |
| Ocular Mel Met to Liver | 0.0 | 0.0 | 0.6 | Liver Tissue 6005-N | 0.0 | 0.0 | 0.0 |
| (ODO4310) | | | | | | | |
| Liver Margin (ODO4310) | 1.0 | 2.0 | 0.0 | Normal Bladder | 4.7 | 2.2 | 2.9 |
| Melanoma Metastasis | 0.0 | 0.0 | 0.0 | Bladder Cancer | 0.0 | 0.0 | 0.0 |
| Lung Margin (ODO4321) | 25.7 | 47.0 | 49.0 | Bladder Cancer | 0.0 | 4.2 | 5.5 |
| Normal Kidney | 86.5 | 100.0 | 100.0 | Bladder Cancer | 0.7 | 1.6 | 1.1 |
| | | | | (ODO4718-01) | | | |
| Kidney Ca, Nuclear grade 2 | 2.2 | 0.0 | 1.1 | Bladder Normal | 4.4 | 0.9 | 6.3 |
| (ODO4338) | | | | Adjacent (ODO4718-03) | | | |
| Kidney Margin (ODO4338) | 55.1 | 35.8 | 58.2 | Normal Ovary | 1.7 | 0.0 | 0.9 |
| Kidney Ca Nuclear grade 1/2 | 0.0 | 0.0 | 0.0 | Ovarian Cancer | 0.0 | 4.2 | 3.3 |
| (ODO4339) | | | | | | | |
| Kidney Margin (ODO4339) | 77.9 | 63.7 | 77.9 | Ovarian Cancer | 0.0 | 0.0 | 0.0 |
| | | | | (ODO4768-07) | | | |
| Kidney Ca, Clear cell type | 1.7 | 0.0 | 0.0 | Ovary Margin | 9.4 | 5.5 | 6.9 |
| (ODO4340) | | | | (ODO4768-08) | | | |
| Kidney Margin (ODO4340) | 100.0 | 53.2 | 62.4 | Normal Stomach | 0.0 | 0.0 | 0.0 |
| Kidney Ca, Nuclear grade 3 | 25.9 | 23.2 | 0.0 | Gastric Cancer 9060358 | 0.0 | 0.0 | 1.5 |
| (ODO4348) | | | | | | | |
| Kidney Margin (ODO4348) | 40.9 | 50.3 | 54.7 | Stomach Margin | 0.0 | 0.0 | 2.0 |
| | | | | 9060359 | | | |
| Kidney Cancer (ODO4622- | 0.6 | 0.0 | 0.0 | Gastric Cancer 9060395 | 0.9 | 1.2 | 1.8 |
| 01) | | | | | | | |
| Kidney Margin (ODO4622- | 0.0 | 0.0 | 1.4 | Stomach Margin | 0.0 | 1.0 | 0.7 |
| 03) | | | | 9060394 | | | |
| Kidney Cancer (ODO4450- | 0.0 | 0.0 | 0.0 | Gastric Cancer 9060397 | 0.0 | 0.0 | 0.0 |
| 01) | | | | | | | |
| Kidney Margin (ODO4450- | 40.3 | 51.1 | 50.7 | Stomach Margin | 0.0 | 0.0 | 0.0 |
| 03) | | | | 9060396 | | | |
| Kidney Cancer 8120607 | 0.0 | 0.0 | 0.0 | Gastric Cancer 064005 | 0.0 | 0.0 | 2.5 |

TABLE LE
Panel 4D
Column A - Rel. Exp. (%) Ag1555, Run 147775116
Column B - Rel. Exp. (%) Ag2315, Run 159202089

| Tissue Name | A | B | Tissue Name | A | B |
|---|---|---|---|---|---|
| Secondary Th1 act | 0.0 | 0.0 | HUVEC IL-1beta | 0.0 | 0.0 |
| Secondary Th2 act | 0.0 | 0.0 | HUVEC IFN gamma | 0.0 | 0.0 |
| Secondary Tr1 act | 0.0 | 0.7 | HUVEC TNF alpha + IFN gamma | 0.0 | 0.0 |
| Secondary Th1 rest | 0.0 | 0.0 | HUVEC TNF alpha +IL4 | 0.0 | 0.0 |
| Secondary Th2 rest | 0.0 | 0.0 | HUVEC IL-11 | 0.0 | 0.0 |
| Secondary Tr1 rest | 0.0 | 0.0 | Lung Microvascular EC none | 0.0 | 0.0 |
| Primary Th1 act | 0.0 | 0.0 | Lung Microvascular EC TNF alpha + | 0.0 | 0.0 |
| | | | IL-1beta | | |
| Primary Th2 act | 0.0 | 0.0 | Microvascular Dermal EC none | 0.0 | 0.0 |
| Primary Tr1 act | 0.0 | 0.0 | Microsvasular Dermal EC TNFalpha | 0.0 | 0.0 |
| | | | + IL-1beta | | |
| Primary Th1 rest | 0.0 | 0.0 | Bronchial epithelium TNFalpha + | 0.0 | 0.0 |
| | | | IL1beta | | |
| Primary Th2 rest | 0.0 | 0.0 | Small airway epithelium none | 0.0 | 0.0 |
| Primary Tr1 rest | 0.0 | 0.0 | Small airway epithelium TNFalpha + | 0.0 | 0.0 |
| | | | IL-1beta | | |
| CD45RA CD4 | 3.3 | 5.0 | Coronery artery SMG rest | 1.0 | 2.3 |
| lymphocyte act | | | | | |
| CD45RO CD4 | 0.0 | 0.0 | Coronery artery SMC TNFalpha + | 3.7 | 1.2 |
| lymphocyte act | | | IL-1beta | | |
| CD8 lymphocyte act | 0.0 | 0.0 | Astrocytes rest | 3.2 | 0.5 |
| Secondary CD8 | 0.0 | 0.0 | Astrocytes TNFalpha + IL-1beta | 1.0 | 1.5 |
| lymphocyte rest | | | | | |
| Secondary CD8 | 0.0 | 0.0 | KU-812 (Basophil) rest | 0.0 | 0.0 |
| lymphocyte act | | | | | |
| CD4 lymphocyte none | 0.0 | 0.0 | KU-812 (Basophil) PMA/ionomycin | 0.0 | 0.0 |
| 2ry Th1/Th2/Tr1 anti- | 0.0 | 0.0 | CCD1106 (Keratinocytes) none | 0.0 | 0.0 |
| CD95 CH11 | | | | | |
| LAK cells rest | 0.0 | 0.0 | 93580 CCD1106 (Keratinocytes) | 0.0 | 0.0 |
| | | | TNFa and IFNg | | |
| LAK cells IL-2 | 0.0 | 0.0 | Liver cirrhosis | 1.4 | 3.8 |
| LAK cells IL-2 + IL-12 | 0.0 | 0.0 | Lupus kidney | 0.0 | 0.8 |
| LAK cells IL-2 + IFN | 0.0 | 0.0 | NCI-H292 none | 0.0 | 0.0 |
| gamma | | | | | |
| LAK cells IL-2 + IL-18 | 0.0 | 0.0 | NCI-H292 IL-4 | 0.0 | 2.3 |
| LAK cells PMA/ | 0.0 | 0.0 | NCI-H292 IL-9 | 0.0 | 0.5 |
| ionomycin | | | | | |
| NK Cells IL-2 rest | 0.0 | 0.0 | NCI-H292 IL-13 | 0.0 | 1.3 |
| Two Way MLR 3 day | 0.0 | 0.0 | NCI-H292 IFN gamma | 0.0 | 0.0 |
| Two Way MLR 5 day | 0.0 | 0.0 | HPAEC none | 0.0 | 0.0 |
| Two Way MLR 7 day | 0.0 | 0.0 | HPAEC TNF alpha + IL-1 beta | 0.0 | 0.0 |
| PBMC rest | 0.0 | 0.0 | Lung fibroblast none | 0.0 | 0.9 |
| PBMC PWM | 0.0 | 0.0 | Lung fibroblast TNF alpha + IL-1 | 0.0 | 0.0 |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 5.7 | 1.3 |
| Ramos (B cell) | 0.0 | 0.0 | Lung fibroblast IL-13 | 1.5 | 1.5 |
| ionomycin | | | | | |
| B lymphocytes PWM | 0.0 | 0.0 | Lung fibroblast IFN gamma | 0.0 | 1.7 |
| B lymphocytes CD40L | 0.0 | 0.0 | Dermal fibroblast CCD1070 rest | 12.9 | 17.2 |
| and IL-4 | | | | | |
| EOL-1 dbcAMP | 0.0 | 0.0 | Dermal fibroblast CCD1070 TNF | 18.6 | 12.0 |
| | | | alpha | | |
| EOL-1 dbcAMP | 0.0 | 0.0 | Dermal fibroblast CCD1070 IL-1 | 6.1 | 2.9 |
| PMA/ionomycin | | | beta | | |
| Dendritic cells none | 0.0 | 0.0 | Dermal fibroblast IFN gamma | 0.0 | 0.0 |
| Dendritic cells LPS | 0.0 | 0.0 | Dermal fibroblast IL-4 | 1.4 | 0.6 |
| Dendritic cells anti-CD40 | 0.0 | 0.0 | IBD Colitis 2 | 0.0 | 1.4 |
| Monocytes rest | 0.0 | 0.0 | IBD Crohn's | 0.0 | 0.0 |
| Monocytes LPS | 0.0 | 0.0 | Colon | 0.6 | 0.0 |
| Macrophages rest | 0.0 | 0.0 | Lung | 4.0 | 11.7 |
| Macrophages LPS | 0.0 | 0.0 | Thymus | 100.0 | 100.0 |
| HUVEC none | 0.0 | 0.0 | Kidney | 4.2 | 5.3 |
| HUVEC starved | 0.0 | 0.0 | | | |

TABLE LF
Panel 5D
Column A - Rel. Exp.(%) Ag2315, Run 169275446

| Tissue Name | A | Tissue Name | A |
|---|---|---|---|
| 97457 Patient-02go adipose | 84.1 | 94709 Donor 2 AM - A adipose | 13.6 |
| 97476 Patient-07sk skeletal muscle | 0.6 | 94710 Donor 2 AM - B adipose | 9.3 |
| 97477 Patient-07ut uterus | 0.0 | 94711 Donor 2 AM - C adipose | 3.6 |
| 97478 Patient-07pl placenta | 7.2 | 94712 Donor 2 AD - A adipose | 8.7 |
| 97481 Patient-08sk skeletal muscle | 4.4 | 94713 Donor 2 AD - B adipose | 17.1 |
| 97482 Patient-08ut uterus | 0.5 | 94714 Donor 2 AD - C adipose | 21.6 |
| 97483 Patient-08pl placenta | 6.5 | 94742 Donor 3 U - A Mesenchymal | 9.0 |
| | | Stem Cells | |
| 97486 Patient-09sk skeletal muscle | 0.0 | 94743 Donor 3 U - B Mesenchymal | 7.3 |
| | | Stem Cells | |
| 97487 Patient-09ut uterus | 0.5 | 94730 Donor 3 AM - A adipose | 14.8 |
| 97488 Patient-09pl placenta | 6.1 | 94731 Donor 3 AM - B adipose | 13.9 |
| 97492 Patient-10ut uterus | 0.0 | 94732 Donor 3 AM - C adipose | 5.9 |
| 97493 Patient-10pl placenta | 7.8 | 94733 Donor 3 AD - A adipose | 5.4 |
| 97495 Patient-11go adipose | 100.0 | 94734 Donor 3 AD - B adipose | 4.7 |
| 97496 Patient-11sk skeletal muscle | 0.6 | 94735 Donor 3 AD - C adipose | 9.3 |
| 97497 Patient-11ut uterus | 1.0 | 77138 Liver HepG2untreated | 6.9 |
| 97498 Patient-11pl placenta | 7.3 | 73556 Heart Cardiac stromal cells | 0.0 |
| | | (primary) | |
| 97500 Patient-12go adipose | 61.6 | 81735 Small Intestine | 1.5 |
| 97501 Patient-12sk skeletal muscle | 3.2 | 72409 Kidney Proximal Convoluted | 0.0 |
| | | Tubule | |
| 97502 Patient-12ut uterus | 1.4 | 82685 Small intestine Duodenum | 0.0 |
| 97503 Patient-12pl placenta | 1.5 | 90650 Adrenal Adrenocortical | 0.0 |
| | | adenoma | |
| 94721 Donor 2 U - A Mesenchymal | 14.4 | 72410 Kidney HRCE | 0.0 |
| Stem Cells | | | |
| 94722 Donor 2 U - B Mesenchymal | 6.7 | 72411 Kidney HRE | 0.0 |
| Stem Cells | | | |
| 94723 Donor 2 U - C Mesenchymal | 6.0 | 73139 Uterus Uterine smooth muscle | 0.0 |
| Stem Cells | | cells | |

Panel 1.3D Summary: Ag1555 Highest expression of this gene was seen in the fetal lung (CTs=32). Modulation of this gene is useful in the treatment of lung related diseases.

Low but significant expression was also seen in the thyroid. Biologic cross-talk between the thyroid and adipose tissue is believed to be a component of some forms of obesity. Modulation of this gene and/or encoded protein is useful in the treatment of endocrine/metabolically related diseases, such as obesity and diabetes.

Panel 2D Summary: Ag1555/2315 Highest expression of this gene was detected in normal kidney tissue (CTs=30.7-32.4). Significant levels of expression of this was also seen in samples derived from normal lung tissue. This gene was preferentially expressed in healthy tissue relative to adjacent cancerous tissue. Modulation of this gene, encoded protein and/or use of small molecule drugs or antibodies targeting this gene is useful in the treatment of kidney cancer and lung cancer.

Panel 4D Summary: Ag1555/Ag2315 This gene was detected at significant levels in the thymus (CT=31.5) and dermal fibroblasts (CT=34). Modulation of this gene, encoded protein and/or use of antibodies or small molecule drug targeting this gene or gene product is useful in maintaining or restoring the normal function to these organs during inflammation.

Panel 5D Summary: Ag2315 This gene showed significant expression in human adipose tissue and in cultured human adipocytes (CT=31-34). Modulation of this gene or gene product is useful in the treatment of obesity.

M. CG50934-03: Mastocytoma Protease Precursor

Expression of full-length physical clone CG50934-03 was assessed using the primer-probe set Ag6974, described in Table MA. Results of the RTQ-PCR runs are shown in Table MB.

TABLE MA
Probe Name Ag6974

| | | | Start | SEQ ID |
|---|---|---|---|---|
| Primers | Sequences | Length | Position | No |
| Forward | 5′-gagacggatggccacag-3′ | 17 | 520 | 1181 |
| Probe | TET-5′-ccaggtggctcagagcagcaggaatgtac- | 29 | 538 | 1182 |
| | 3′-TAMRA | | | |
| Reverse | 5′-cgttccgcctgcagag-3′ | 16 | 577 | 1183 |

TABLE MB
General_screening_panel_v1.6
Column A - Rel. Exp.(%) Ag6974, Run 278389211

| Tissue Name | A | Tissue Name | A |
|---|---|---|---|
| Adipose | 0.0 | Renal ca. TK-10 | 0.0 |
| Melanoma* | 0.0 | Bladder | 0.2 |
| Hs688(A).T | | | |
| Melanoma* | 0.0 | Gastric ca. (liver met.) NCI-N87 | 34.4 |
| Hs688(B).T | | | |
| Melanoma* M14 | 0.0 | Gastric ca. KATO III | 0.0 |
| Melanoma* LOXIMVI | 0.0 | Colon ca. SW-948 | 0.0 |
| Melanoma* | 0.0 | Colon ca. SW480 | 1.2 |
| SK-MEL-5 | | | |
| Squamous cell | 0.0 | Colon ca.* (SW480 met) SW620 | 1.8 |
| carcinoma SCC-4 | | | |
| Testis Pool | 0.0 | Colon ca. HT29 | 0.2 |
| Prostate ca.* | 0.0 | Colon ca. HCT-116 | 0.0 |
| (bone met) PC-3 | | | |
| Prostate Pool | 0.0 | Colon ca. CaCo-2 | 0.9 |
| Placenta | 0.6 | Colon cancer tissue | 11.9 |
| Uterus Pool | 0.0 | Colon ca. SW1116 | 0.0 |
| Ovarian ca. OVCAR-3 | 0.0 | Colon ca. Colo-205 | 2.7 |
| Ovarian ca. SK-OV-3 | 0.0 | Colon ca. SW-48 | 0.0 |
| Ovarian ca. OVCAR-4 | 0.1 | Colon Pool | 0.3 |
| Ovarian ca. OVCAR-5 | 100.0 | Small Intestine Pool | 0.3 |
| Ovarian ca. IGROV-1 | 0.0 | Stomach Pool | 0.0 |
| Ovarian ca. OVCAR-8 | 0.0 | Bone Marrow Pool | 0.1 |
| Ovary | 0.0 | Fetal Heart | 0.0 |
| Breast ca. MCF-7 | 9.3 | Heart Pool | 0.0 |
| Breast ca. | 0.0 | Lymph Node Pool | 0.2 |
| MDA-MB-231 | | | |
| Breast ca. BT 549 | 0.0 | Fetal Skeletal Muscle | 0.0 |
| Breast ca. T47D | 0.4 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) U87-MG | 0.0 |
| Lung | 0.0 | CNS cancer (glio/astro) U-118-MG | 0.2 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 21.5 | CNS cancer (astro) SNB-75 | 0.0 |
| Lung ca. NCI-H146 | 0.0 | CNS cancer (glio) SNB-19 | 0.0 |
| Lung ca. SHP-77 | 4.0 | CNS cancer (glio) SF-295 | 0.5 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.2 | Brain (fetal) | 0.2 |
| Lung ca. NCI-H460 | 0.0 | Brain (Hippocampus) Pool | 0.0 |
| Lung ca. HOP-62 | 0.0 | Cerebral Cortex Pool | 0.1 |
| Lung ca. NCI-H522 | 0.0 | Brain (Substantia nigra) Pool | 0.0 |
| Liver | 0.0 | Brain (Thalamus) Pool | 0.0 |
| Fetal Liver | 1.5 | Brain (whole) | 0.0 |
| Liver ca. HepG2 | 1.1 | Spinal Cord Pool | 0.0 |
| Kidney Pool | 0.1 | Adrenal Gland | 0.0 |
| Fetal Kidney | 0.4 | Pituitary gland Pool | 1.3 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 8.5 |
| Renal ca. UO-31 | 0.0 | Pancreas Pool | 0.0 |

General_screening_panel_v1.6 Summary: Ag6974 Highest expression of this gene was detected in an ovarian cancer OVCAR-5 cell line (CT=28). This gene showed preferential expression in colon cancer tissue and a number of cancer cell lines derived from pancreatic, colon, gastric, lung, breast and ovarian cancers. Expression of this gene is useful as diagnostic marker to detect these cancers and also, modulation of this gene, encoded protein and/or use of antibodies or small molecule drug targeting this gene or gene product is useful in the treatment of these cancers.

N. CG51213-01 and CG51213-04: Zinc Metalloendopeptidase

Expression of gene CG51213-01 and CG51213-04 was assessed using the primer-probe sets Ag813, and Ag3985, described in Tables NA and NB. Results of the RTQ-PCR runs are shown in Tables NC, ND NE and NF. Please note that the primer-probe set Ag3985 is specific for CG51213-04 only.

TABLE NA
Probe Name Ag813

| | | | Start | SEQ ID |
|---|---|---|---|---|
| Primers | Sequences | Length | Position | No |
| Forward | 5′-tgtagaatttcccacggaaag-3′ | 21 | 1185 | 1184 |
| Probe | TET-5′-cactgcacttctctgaagtcctggga-3′- | 26 | 1139 | 1185 |
| | TAMRA | | | |
| Reverse | 5′-ctgcaacacggatgactgt-3′ | 19 | 1111 | 1186 |

TABLE NB
Probe Name Ag3985

| | | | Start | SEQ ID |
|---|---|---|---|---|
| Primers | Sequences | Length | Position | No |
| Forward | 5′-tgggaaattctacaagtggaaa-3′ | 22 | 1192 | 1187 |
| Probe | TET-5′-ctcgctcacgagcctagcggaag-3′- | 23 | 1243 | 1188 |
| | TAMRA | | | |
| Reverse | 5′-cctctccgtgtagaagttgaag-3′ | 22 | 1267 | 1189 |

TABLE NC
AI_comprehensive panel_v1.0
Column A - Rel. Exp.(%) Ag3985, Run 226203363
Column B - Rel. Exp.(%) Ag813, Run 234222162
Column C - Rel. Exp.(%) Ag813, Run 246953625

| Tissue Name | A | B | C | Tissue Name | A | B | C |
|---|---|---|---|---|---|---|---|
| 110967 COPD-F | 14.1 | 5.4 | 8.8 | 112427 Match Control | 31.6 | 0.0 | 30.6 |
| | | | | Psoriasis-F | | | |
| 110980 COPD-F | 8.5 | 5.9 | 9.2 | 112418 Psoriasis-M | 8.8 | 8.6 | 8.7 |
| 110968 COPD-M | 18.4 | 12.9 | 11.9 | 112723 Match Control | 8.9 | 11.0 | 8.8 |
| | | | | Psoriasis-M | | | |
| 110977 COPD-M | 0.0 | 18.8 | 25.7 | 112419 Psoriasis-M | 4.7 | 10.7 | 8.1 |
| 110989 Emphysema-F | 37.1 | 19.3 | 26.4 | 112424 Match Control | 5.0 | 7.4 | 4.1 |
| | | | | Psoriasis-M | | | |
| 110992 Emphysema-F | 22.1 | 13.5 | 30.8 | 112420 Psoriasis-M | 40.3 | 37.4 | 36.3 |
| 110993 Emphysema-F | 22.8 | 10.5 | 13.2 | 112425 Match Control | 16.2 | 11.7 | 6.2 |
| | | | | Psoriasis-M | | | |
| 110994 Emphysema-F | 10.2 | 10.4 | 7.3 | 104689 (MF) OA Bone- | 94.6 | 100.0 | 100.0 |
| | | | | Backus | | | |
| 110995 Emphysema-F | 60.3 | 25.5 | 25.9 | 104690 (MF) Adj “Normal” | 90.1 | 62.0 | 65.5 |
| | | | | Bone-Backus | | | |
| 110996 Emphysema-F | 7.0 | 3.7 | 6.5 | 104691 (ME) OA | 100.0 | 73.7 | 74.7 |
| | | | | Synovium-Backus | | | |
| 110997 Asthma-M | 4.1 | 2.5 | 2.4 | 104692 (BA) OA Cartilage- | 24.5 | 15.8 | 15.0 |
| | | | | Backus | | | |
| 111001 Asthma-F | 21.8 | 16.3 | 21.0 | 104694 (BA) OA Bone- | 85.9 | 69.3 | 79.0 |
| | | | | Backus | | | |
| 111002 Asthma-F | 27.9 | 24.0 | 22.1 | 104695 (BA) Adj “Normal” | 78.5 | 68.3 | 44.1 |
| | | | | Bone-Back | | | |
| 111003 Atopic Asthma-F | 25.5 | 14.9 | 35.4 | 104696 (BA) OA | 33.9 | 29.5 | 27.9 |
| | | | | Synovium-Backus | | | |
| 111004 Atopic Asthma-F | 46.0 | 31.6 | 147.0 | 104700 (SS) OA Bone- | 42.3 | 55.1 | 43.2 |
| | | | | Backus | | | |
| 111005 Atopic Asthma-F | 27.4 | 18.4 | 20.2 | 104701 (SS) Adj “Normal” | 75.3 | 72.2 | 95.3 |
| | | | | Bone-Backus | | | |
| 111006 Atopic Asthma-F | 8.4 | 2.6 | 5.6 | 104702 (SS) OA | 48.0 | 36.3 | 37.9 |
| | | | | Synovium-Backus | | | |
| 111417 Allergy-M | 21.8 | 13.4 | 8.5 | 117093 OA Cartilage Rep 7 | 7.5 | 4.9 | 11.3 |
| 112347 Allergy-M | 0.5 | 0.0 | 0.0 | 112672 OA Bone5 | 34.6 | 25.3 | 25.0 |
| 112349 Normal Lung-F | 0.0 | 0.0 | 0.0 | 112673 OA Synovium5 | 12.3 | 8.4 | 12.6 |
| 112357 Normal Lung-F | 25.2 | 15.5 | 16.4 | 112674 OA Synovial Fluid | 21.2 | 18.8 | 16.2 |
| | | | | cells5 | | | |
| 112354 Normal Lung-M | 6.2 | 3.7 | 1.5 | 117100 OA Cartilage | 11.8 | 8.0 | 10.5 |
| | | | | Rep14 | | | |
| 112374 Crohns-F | 12.2 | 16.6 | 21.6 | 112756 OA Bone9 | 7.7 | 3.6 | 11.2 |
| 112389 Match Control | 9.3 | 10.3 | 6.3 | 112757 OA Synovium9 | 5.4 | 6.0 | 5.4 |
| Crohns-F | | | | | | | |
| 112375 Crohns-F | 19.5 | 0.0 | 32.8 | 112758 OA Synovial Fluid | 12.2 | 9.9 | 9.4 |
| | | | | Cells9 | | | |
| 112732 Match Control | 15.4 | 10.4 | 9.7 | 117125 RA Cartilage Rep2 | 12.4 | 5.3 | 9.3 |
| Crohns-F | | | | | | | |
| 112725 Crohns-M | 1.0 | 2.2 | 0.8 | 113492 Bone2 RA | 6.8 | 4.0 | 4.1 |
| 112387 Match Control | 12.9 | 8.4 | 10.5 | 113493 Synovium2 RA | 0.2 | 1.0 | 1.7 |
| Crohns-M | | | | | | | |
| 112378 Crohns-M | 0.5 | 0.0 | 0.0 | 113494 Syn Fluid Cells RA | 3.5 | 2.6 | 5.6 |
| 112390 Match Control | 50.0 | 38.7 | 38.2 | 113499 Cartilage4 RA | 5.5 | 4.7 | 5.2 |
| Crohns-M | | | | | | | |
| 112726 Crohns-M | 31.2 | 27.4 | 22.8 | 113500 Bone4 RA | 6.4 | 4.0 | 4.6 |
| 112731 Match Control | 23.5 | 7.6 | 13.6 | 113501 Synovium4 RA | 4.2 | 3.6 | 3.1 |
| Crohns-M | | | | | | | |
| 112380 Ulcer Col-F | 18.6 | 15.9 | 20.4 | 113502 Syn Fluid Cells4 | 2.6 | 2.3 | 1.9 |
| | | | | RA | | | |
| 112734 Match Control | 28.3 | 13.5 | 26.4 | 113495 Cartilage3 RA | 1.3 | 3.3 | 5.4 |
| Ulcer Col-F | | | | | | | |
| 112384 Ulcer Col-F | 47.0 | 21.6 | 18.8 | 113496 Bone3 RA | 0.9 | 4.6 | 6.4 |
| 112737 Match Control | 8.5 | 5.6 | 5.8 | 113497 Synovium3 RA | 1.1 | 3.1 | 1.6 |
| Ulcer Col-F | | | | | | | |
| 112386 Ulcer Col-F | 8.6 | 0.7 | 1.1 | 113498 Syn Fluid Cells3 | 4.9 | 6.9 | 6.0 |
| | | | | RA | | | |
| 112738 MatchControl | 3.6 | 3.0 | 4.3 | 117106 Normal Cartilage | 16.4 | 13.7 | 13.0 |
| Ulcer Col-F | | | | Rep20 | | | |
| 112381 Ulcer Col-M | 2.5 | 0.0 | 0.1 | 113663 Bone3 Normal | 0.0 | 0.0 | 0.0 |
| 112735 Match Control | 2.2 | 2.1 | 0.8 | 113664 Synovium3 Normal | 0.0 | 0.0 | 0.0 |
| Ulcer Col-M | | | | | | | |
| 112382 Ulcer Col-M | 3.7 | 8.7 | 8.5 | 113665 Syn Fluid Cells3 | 0.0 | 0.0 | 0.1 |
| | | | | Normal | | | |
| 112394 Match Control | 3.7 | 1.5 | 4.7 | 117107 Normal Cartilage | 2.3 | 2.3 | 0.3 |
| Ulcer Col-M | | | | Rep22 | | | |
| 112383 Ulcer Col-M | 52.5 | 45.7 | 54.7 | 113667 Bone4 Normal | 11.0 | 8.6 | 4.7 |
| 112736 Match Control | 3.2 | 6.1 | 6.4 | 113668 Synovium4 Normal | 2.9 | 3.0 | 6.4 |
| Ulcer Col-M | | | | | | | |
| 112423 Psoriasis-F | 9.2 | 7.7 | 5.2 | 113669 Syn Fluid Cells4 | 13.9 | 12.7 | 11.0 |
| | | | | Normal | | | |

TABLE ND
General_screening_panel_v1.5
Column A - Rel. Exp.(%) Ag813, Run 247945092

| Tissue Name | A | Tissue Name | A |
|---|---|---|---|
| Adipose | 15.2 | Renal ca. TK-10 | 50.3 |
| Melanoma* | 26.2 | Bladder | 88.3 |
| Hs688(A).T | | | |
| Melanoma* | 42.6 | Gastric ca. (liver met.) NCI-N87 | 0.0 |
| Hs688(B).T | | | |
| Melanoma* M14 | 0.0 | Gastric ca. KATO III | 0.0 |
| Melanoma* LOXIMVI | 4.7 | Colon ca. SW-948 | 0.0 |
| Melanoma* | 0.0 | Colon ca. SW480 | 0.3 |
| SK-MEL-5 | | | |
| Squamous cell | 0.0 | Colon ca.* (SW480 met) SW620 | 0.0 |
| carcinoma SCC-4 | | | |
| Testis Pool | 9.6 | Colon ca. HT29 | 0.0 |
| Prostate ca.* | 0.0 | Colon ca. HCT-116 | 3.2 |
| (bone met) PC-3 | | | |
| Prostate Pool | 4.6 | Colon ca. CaCo-2 | 0.4 |
| Placenta | 36.6 | Colon cancer tissue | 30.1 |
| Uterus Pool | 5.4 | Colon ca. SW1116 | 0.0 |
| Ovarian ca. OVCAR-3 | 0.0 | Colon ca. Colo-205 | 0.0 |
| Ovarian ca. SK-OV-3 | 1.9 | Colon ca. SW-48 | 0.0 |
| Ovarian ca. OVCAR-4 | 1.2 | Colon Pool | 29.7 |
| Ovarian ca. OVCAR-5 | 14.3 | Small Intestine Pool | 9.5 |
| Ovarian ca. IGROV-1 | 9.7 | Stomach Pool | 16.3 |
| Ovarian ca. OVCAR-8 | 24.1 | Bone Marrow Pool | 7.9 |
| Ovary | 20.6 | Fetal Heart | 11.8 |
| Breast ca. MCF-7 | 0.0 | Heart Pool | 10.9 |
| Breast ca. | 0.0 | Lymph Node Pool | 31.4 |
| MDA-MB-231 | | | |
| Breast ca. BT 549 | 15.7 | Fetal Skeletal Muscle | 15.7 |
| Breast ca. T47D | 1.0 | Skeletal Muscle Pool | 4.1 |
| Breast ca. MDA-N | 0.0 | Spleen Pool | 12.3 |
| Breast Pool | 30.1 | Thymus Pool | 37.1 |
| Trachea | 6.0 | CNS cancer (glio/astro) U87-MG | 0.0 |
| Lung | 4.9 | CNS cancer (glio/astro) U-118-MG | 0.7 |
| Fetal Lung | 59.5 | CNS cancer (neuro; met) SK-N-AS | 0.6 |
| Lung ca. NCI-N417 | 0.0 | CNS cancer (astro) SF-539 | 15.0 |
| Lung ca. LX-1 | 0.0 | CNS cancer (astro) SNB-75 | 100.0 |
| Lung ca. NCI-H146 | 0.0 | CNS cancer (glio) SNB-19 | 10.7 |
| Lung ca. SHP-77 | 1.5 | CNS cancer (glio) SF-295 | 14.8 |
| Lung ca. A549 | 75.3 | Brain (Amygdala) Pool | 13.7 |
| Lung ca. NCI-H526 | 0.0 | Brain (cerebellum) | 8.5 |
| Lung ca. NCI-H23 | 30.6 | Brain (fetal) | 95.9 |
| Lung ca. NCI-H460 | 0.3 | Brain (Hippocampus) Pool | 12.9 |
| Lung ca. HOP-62 | 19.9 | Cerebral Cortex Pool | 20.0 |
| Lung ca. NCI-H522 | 17.7 | Brain (Substantia nigra) Pool | 10.5 |
| Liver | 0.4 | Brain (Thalamus) Pool | 22.2 |
| Fetal Liver | 6.3 | Brain (whole) | 12.0 |
| Liver ca. HepG2 | 0.0 | Spinal Cord Pool | 21.0 |
| Kidney Pool | 36.6 | Adrenal Gland | 19.2 |
| Fetal Kidney | 36.6 | Pituitary gland Pool | 1.6 |
| Renal ca. 786-0 | 0.0 | Salivary Gland | 0.4 |
| Renal ca. A498 | 55.5 | Thyroid (female) | 4.0 |
| Renal ca. ACHN | 0.0 | Pancreatic ca. CAPAN2 | 1.1 |
| Renal ca. UO-31 | 7.0 | Pancreas Pool | 45.7 |

TABLE NE
Panel 4.1D
Column A - Rel. Exp. (%) Ag3985, Run 170721255
Column B - Rel. Exp. (%) Ag813, Run 237369996

| Tissue Name | A | B | Tissue Name | A | B |
|---|---|---|---|---|---|
| Secondary Th1 act | 6.7 | 4.8 | HUVEC IL-1beta | 6.0 | 11.6 |
| Secondary Th2 act | 21.3 | 11.7 | HUVEC IFN gamma | 23.2 | 6.9 |
| Secondary Tr1 act | 14.8 | 5.3 | HUVEC TNF alpha + IFN gamma | 10.4 | 2.7 |
| Secondary Th1 rest | 33.2 | 4.9 | HUVEC TNF alpha +IL4 | 4.9 | 1.3 |
| Secondary Th2 rest | 46.3 | 2.1 | HUVEC IL-11 | 2.9 | 20.0 |
| Secondary Tr1 rest | 61.6 | 0.0 | Lung Microvascular EC none | 100.0 | 95.9 |
| Primary Th1 act | 27.2 | 0.0 | Lung Microvascular EC TNF alpha + | 54.3 | 39.0 |
| | | | IL-1beta | | |
| Primary Th2 act | 17.2 | 61.1 | Microvascular Dermal EC none | 23.7 | 2.8 |
| Primary Tr1 act | 12.4 | 25.9 | Microsvasular Dermal EC TNFalpha | 15.2 | 6.7 |
| | | | + IL-1beta | | |
| Primary Th1 rest | 40.1 | 14.7 | Bronchial epithelium TNFalpha + | 0.0 | 0.0 |
| | | | IL1beta | | |
| Primary Th2 rest | 28.1 | 15.8 | Small airway epithelium none | 11.9 | 0.0 |
| Primary Tr1 rest | 59.9 | 4.1 | Small airway epithelium TNFalpha + | 0.0 | 0.0 |
| | | | IL-1beta | | |
| CD45RA CD4 | 10.9 | 5.4 | Coronery artery SMG rest | 23.5 | 33.4 |
| lymphocyte act | | | | | |
| CD45RO CD4 | 9.9 | 22.2 | Coronery artery SMC TNFalpha + | 43.8 | 31.4 |
| lymphocyte act | | | IL-1beta | | |
| CD8 lymphocyte act | 14.3 | 0.9 | Astrocytes rest | 83.5 | 20.4 |
| Secondary CD8 | 0.0 | 6.0 | Astrocytes TNFalpha + IL-1beta | 63.3 | 6.9 |
| lymphocyte rest | | | | | |
| Secondary CD8 | 17.4 | 0.0 | KU-812 (Basophil) rest | 0.0 | 0.0 |
| lymphocyte act | | | | | |
| CD4 lymphocyte none | 27.4 | 6.0 | KU-812 (Basophil) PMA/ionomycin | 2.6 | 11.0 |
| 2ry Th1/Th2/Tr1 anti- | 74.7 | 7.1 | CCD1106 (Keratinocytes) none | 0.0 | 0.0 |
| CD95 CH11 | | | | | |
| LAK cells rest | 8.8 | 11.3 | 93580 CCD1106 (Keratinocytes) | 0.0 | 0.0 |
| | | | TNFa and IFNg | | |
| LAK cells IL-2 | 22.7 | 0.0 | Liver cirrhosis | 27.9 | 32.5 |
| LAK cells IL-2 + IL-12 | 8.4 | 0.0 | Lupus kidney | 0.0 | 0.0 |
| LAK cells IL-2 + IFN | 11.0 | 0.0 | NCI-H292 none | 0.0 | 0.0 |
| gamma | | | | | |
| LAK cells IL-2 + IL-18 | 12.2 | 0.0 | NCI-H292 IL-4 | 0.0 | 0.0 |
| LAK cells PMA/ | 13.0 | 10.7 | NCI-H292 IL-9 | 0.0 | 5.4 |
| ionomycin | | | | | |
| NK Cells IL-2 rest | 47.3 | 100.0 | NCI-H292 IL-13 | 0.0 | 0.0 |
| Two Way MLR 3 day | 48.0 | 13.9 | NCI-H292 IFN gamma | 14.5 | 5.3 |
| Two Way MLR 5 day | 17.7 | 0.0 | HPAEC none | 2.6 | 4.9 |
| Two Way MLR 7 day | 5.5 | 0.0 | HPAEC TNF alpha + IL-1 beta | 11.2 | 10.7 |
| PBMC rest | 38.7 | 2.9 | Lung fibroblast none | 2.6 | 9.4 |
| PBMC PWM | 14.5 | 0.0 | Lung fibroblast TNF alpha + IL-1 | 7.9 | 7.8 |
| | | | beta | | |
| PBMC PHA-L | 0.0 | 0.0 | Lung fibroblast IL-4 | 6.0 | 6.8 |
| Ramos (B cell) none | 0.0 | 0.0 | Lung fibroblast IL-9 | 22.8 | 0.0 |
| Ramos (B cell) | 0.0 | 0.0 | Lung fibroblast IL-13 | 7.5 | 16.5 |
| ionomycin | | | | | |
| B lymphocytes PWM | 0.0 | 2.4 | Lung fibroblast IFN gamma | 11.9 | 12.2 |
| B lymphocytes CD40L | 7.1 | 2.9 | Dermal fibroblast CCD1070 rest | 7.3 | 25.5 |
| and IL-4 | | | | | |
| EOL-1 dbcAMP | 48.3 | 80.1 | Dermal fibroblast CCD1070 TNF | 10.7 | 20.3 |
| | | | alpha | | |
| EOL-1 dbcAMP | 60.7 | 0.0 | Dermal fibroblast CCD1070 IL-1 | 29.9 | 9.9 |
| PMA/ionomycin | | | beta | | |
| Dendritic cells none | 4.2 | 0.0 | Dermal fibroblast IFN gamma | 59.9 | 57.8 |
| Dendritic cells LPS | 0.0 | 0.0 | Dermal fibroblast IL-4 | 14.3 | 13.0 |
| Dendritic cells anti-CD40 | 0.0 | 0.0 | IBD Colitis 2 | 3.3 | 0.0 |
| Monocytes rest | 0.0 | 0.0 | IBD Crohn's | 0.0 | 2.5 |
| Monocytes LPS | 0.0 | 0.0 | Colon | 9.0 | 0.0 |
| Macrophages rest | 4.7 | 0.0 | Lung | 4.5 | 0.0 |
| Macrophages LPS | 0.0 | 0.0 | Thymus | 15.6 | 4.8 |
| HUVEC none | 0.6 | 0.0 | Kidney | 11.3 | 21.9 |
| HUVEC starved | 8.7 | 14.2 | | | |

TABLE NF
Panel 5 Islet
Column A - Rel. Exp.(%) Ag813, Run 254387841

| Tissue Name | A | Tissue Name | A |
|---|---|---|---|
| 97457 Patient-02go adipose | 45.7 | 94709 Donor 2 AM - A adipose | 49.3 |
| 97476 Patient-07sk skeletal muscle | 11.7 | 94710 Donor 2 AM - B adipose | 15.8 |
| 97477 Patient-07ut uterus | 33.2 | 94711 Donor 2 AM - C adipose | 8.4 |
| 97478 Patient-07pl placenta | 11.7 | 94712 Donor 2 AD - A adipose | 52.9 |
| 99167 Bayer Patient 1 | 14.4 | 94713 Donor 2 AD - B adipose | 36.3 |
| 97482 Patient-08ut uterus | 45.7 | 94714 Donor 2 AD - C adipose | 35.6 |
| 97483 Patient-08pl placenta | 7.0 | 94742 Donor 3 U - A Mesenchymal | 27.4 |
| | | Stem Cells | |
| 97486 Patient-09sk skeletal muscle | 0.0 | 94743 Donor 3 U - B Mesenchymal | 33.9 |
| | | Stem Cells | |
| 97487 Patient-09ut uterus | 16.3 | 94730 Donor 3 AM - A adipose | 17.2 |
| 97488 Patient-09pl placenta | 13.8 | 94731 Donor 3 AM - B adipose | 21.2 |
| 97492 Patient-10ut uterus | 24.3 | 94732 Donor 3 AM - C adipose | 4.9 |
| 97493 Patient-10pl placenta | 5.1 | 94733 Donor 3 AD - A adipose | 100.0 |
| 97495 Patient-11go adipose | 9.7 | 94734 Donor 3 AD - B adipose | 40.3 |
| 97496 Patient-11sk skeletal muscle | 15.0 | 94735 Donor 3 AD - C adipose | 69.7 |
| 97497 Patient-11ut uterus | 43.2 | 77138 Liver HepG2untreated | 0.0 |
| 97498 Patient-11pl placenta | 7.9 | 73556 Heart Cardiac stromal cells | 7.9 |
| | | (primary) | |
| 97500 Patient-12go adipose | 36.3 | 81735 Small Intestine | 54.3 |
| 97501 Patient-12sk skeletal muscle | 33.2 | 72409 Kidney Proximal Convoluted | 0.0 |
| | | Tubule | |
| 97502 Patient-12ut uterus | 55.1 | 82685 Small intestine Duodenum | 0.0 |
| 97503 Patient-12pl placenta | 0.0 | 90650 Adrenal Adrenocortical | 26.2 |
| | | adenoma | |
| 94721 Donor 2 U - A Mesenchymal | 66.0 | 72410 Kidney HRCE | 0.0 |
| Stem Cells | | | |
| 94722 Donor 2 U - B Mesenchymal | 32.1 | 72411 Kidney HRE | 0.0 |
| Stem Cells | | | |
| 94723 Donor 2 U - C Mesenchymal | 62.0 | 73139 Uterus Uterine smooth muscle | 6.3 |
| Stem Cells | | cells | |

AI_comprehensive panel_v1.0 Summary: Ag3985/Ag813 Highest expression of this gene was detected in samples from an osteoarthritic bone sample and synovium (CTs=30). Significant expression of this gene was detected in samples derived from orthoarthitis bone, cartilage, synovium and synovial fluid samples, from normal lung, COPD lung, emphysema, atopic asthma, asthma, allergy, Crohn's disease (normal matched control and diseased), ulcerative colitis (normal matched control and diseased), and psoriasis (normal matched control and diseased). Modulation of this gene, encoded protein and/or use of antibodies or small molecule drug targeting this gene or gene product is useful for the amelioration of symptoms/conditions associated with autoimmune and inflammatory disorders including psoriasis, allergy, asthma, inflammatory bowel disease, and osteoarthritis.

General_screening_panel_v1.5 Summary: Ag813 Highest expression of this gene was detected in fetal brain and a brain cancer SNB-75 cell line (CTs=31). In addition, moderate expression of this gene was seen in all regions of the central nervous system examined, including amygdala, hippocampus, substantia nigra, thalamus, cerebellum, cerebral cortex, and spinal cord. This gene codes for a variant of ADAMTS-10, a member of Matrix metalloproteinases (MMPs). MMPs are a gene family of neutral proteases that are important in normal development, wound healing, and a wide variety of pathological processes, including the spread of metastatic cancer cells, arthritic destruction of joints, atherosclerosis, and neuroinflammation. In the central nervous system (CNS), MMPs have been shown to degrade components of the basal lamina, leading to disruption of the blood-brain barrier (BBB), and to contribute to the neuroinflammatory response in many neurological diseases (Rosenberg G A, 2002, Glia 39(3):279-91, PMID: 12203394). Modulation of this gene, encoded protein and/or use of antibodies of small molecule drug targeting this gene or gene product is useful in the treatment of neurological disorders such as Alzheimer's disease, Parkinson's disease, epilepsy, multiple sclerosis, schizophrenia, depression, allergic encephalomyelitis (EAE), allergic neuritis (EAN), and cerebral ischemia.

Moderate to low levels of expression of this gene were also detected in tissues with metabolic/endocrine function including pancreas, adipose, adrenal gland, skeletal muscle, heart, fetal liver and the gastrointestinal tract. Modulation of this gene, encoded protein and/or use of antibodies or small molecule drug targeting this gene or gene product is useful in the treatment of endocrine/metabolically related diseases, such as obesity and diabetes.

This gene was expressed at moderate to low levels in number of cancer cell lines derived from melanoma, ovarian, breast, lung, renal, colon and brain cancers. Modulation of this gene, encoded protein and/or use of antibodies or small molecule drug targeting this gene or gene product is useful in the treatment of these cancers.

Panel 4.1D Summary: Ag813/Ag3985 Highest expression of this gene was detected in IL-2 treated resting NK cells and lung microvascular endothelial cells (CTs=31-32.8). Moderate to low levels of expression of this gene were also detected in activated primary polarized T cells, eosinophils, lung microvascular endothelial cells, coronery artery SMC, liver cirrhosis and activated dermal fibroblasts. Modulation of this gene, encoded protein and/or use of antibodies or small molecule drug targeting this gene or gene product is useful in the treatment of autoimmune and inflammatory diseases including asthma, allergies, inflammatory bowel disease, lupus erythematosus, psoriasis, rheumatoid arthritis, and osteoarthritis.

Panel 5 Islet Summary: Ag813 Highest expression of this gene was detected in differentiated adipose cells (CT=33.5). Low expression of this gene was seen mainly in adipose and small intestine. Therefore, modulation of this gene and/or encoded protein is useful in the treatment of obesity and diabetes, including Type II diabetes.

O. CG51448-01 and CG51448-05: Myosin, Light Polypeptide Kinase

Expression of gene CG51448-05 was assessed using the primer-probe sets Ag1289 and Ag764, described in Tables OA and OB. Results of the RTQ-PCR runs are shown in Tables OC, OD and OE.

TABLE OA
Probe Name Ag1289

| | | | Start | SEQ ID |
|---|---|---|---|---|
| Primers | Sequences | Length | Position | No |
| Forward | 5′-aacgagaagctgaaggtgaact-3′ | 22 | 1277 | 1190 |
| Probe | TET-5′-accccagagttcctgtcacctgaggt-3′- | 26 | 1304 | 1191 |
| | TAMRA | | | |
| Reverse | 5′-tcggagatttggtcataattca-3′ | 22 | 1332 | 1192 |

TABLE OB
Probe Name Ag764

| | | | Start | SEQ ID |
|---|---|---|---|---|
| Primers | Sequences | Length | Position | No |
| Forward | 5′-aacgagaagctgaaggtgaact-3′ | 22 | 1277 | 1193 |
| Probe | TET-5′-accccagagttcctgtcacctgaggt-3′- | 26 | 1304 | 1194 |
| | TAMRA | | | |
| Reverse | 5′-tcggagatttggtcataattca-3′ | 22 | 1332 | 1195 |

TABLE OC
AI_comprehensive panel_v1.0
Column A - Rel. Exp.(%) Ag1289, Run 219421496
Column B - Rel. Exp.(%) Ag1289, Run 224054768
Column C - Rel. Exp.(%) Ag764, Run 219421440

| Tissue Name | A | B | C | Tissue Name | A | B | C |
|---|---|---|---|---|---|---|---|
| 110967 COPD-F | 0.0 | 0.0 | 0.0 | 112427 Match Control | 0.1 | 0.0 | 0.2 |
| | | | | Psoriasis-F | | | |
| 110980 COPD-F | 0.5 | 0.7 | 1.0 | 112418 Psoriasis-M | 0.0 | 0.0 | 0.0 |
| 110968 COPD-M | 0.0 | 0.1 | 0.0 | 112723 Match Control | 0.1 | 0.1 | 0.0 |
| | | | | Psoriasis-M | | | |
| 110977 COPD-M | 0.8 | 0.9 | 1.5 | 112419 Psoriasis-M | 0.0 | 0.1 | 0.0 |
| 110989 Emphysema-F | 0.4 | 0.1 | 0.2 | 112424 Match Control | 0.0 | 0.0 | 0.2 |
| | | | | Psoriasis-M | | | |
| 110992 Emphysema-F | 0.4 | 0.3 | 0.4 | 112420 Psoriasis-M | 0.2 | 0.1 | 0.0 |
| 110993 Emphysema-F | 0.4 | 0.3 | 0.2 | 112425 Match Control | 0.1 | 0.3 | 0.2 |
| | | | | Psoriasis-M | | | |
| 110994 Emphysema-F | 0.1 | 0.0 | 0.2 | 104689 (MF) OA Bone- | 0.6 | 0.7 | 0.6 |
| | | | | Backus | | | |
| 110995 Emphysema-F | 0.7 | 0.5 | 0.4 | 104690 (MF) Adj “Normal” | 0.0 | 0.2 | 0.2 |
| | | | | Bone-Backus | | | |
| 110996 Emphysema-F | 0.1 | 0.1 | 1.3 | 104691 (ME) OA | 0.6 | 0.3 | 1.5 |
| | | | | Synovium-Backus | | | |
| 110997 Asthma-M | 0.2 | 0.0 | 0.0 | 104692 (BA) OA Cartilage- | 0.0 | 0.0 | 0.0 |
| | | | | Backus | | | |
| 111001 Asthma-F | 0.0 | 0.2 | 0.0 | 104694 (BA) OA Bone- | 0.1 | 0.2 | 0.5 |
| | | | | Backus | | | |
| 111002 Asthma-F | 0.2 | 0.0 | 0.7 | 104695 (BA) Adj “Normal” | 0.2 | 0.0 | 0.4 |
| | | | | Bone-Back | | | |
| 111003 Atopic Asthma-F | 0.2 | 0.0 | 0.2 | 104696 (BA) OA | 0.0 | 0.1 | 0.0 |
| | | | | Synovium-Backus | | | |
| 111004 Atopic Asthma-F | 0.8 | 0.9 | 1.3 | 104700 (SS) OA Bone- | 0.1 | 0.1 | 0.2 |
| | | | | Backus | | | |
| 111005 Atopic Asthma-F | 0.4 | 0.5 | 0.6 | 104701 (SS) Adj “Normal” | 0.1 | 0.2 | 0.2 |
| | | | | Bone-Backus | | | |
| 111006 Atopic Asthma-F | 0.1 | 0.3 | 0.2 | 104702 (SS) OA | 0.0 | 0.2 | 0.2 |
| | | | | Synovium-Backus | | | |
| 111417 Allergy-M | 0.2 | 0.0 | 0.0 | 117093 OA Cartilage Rep 7 | 0.0 | 0.0 | 0.2 |
| 112347 Allergy-M | 0.1 | 0.1 | 0.0 | 112672 OA Bone5 | 0.3 | 0.2 | 0.5 |
| 112349 Normal Lung-F | 0.0 | 0.1 | 0.1 | 112673 OA Synovium5 | 0.2 | 0.0 | 0.3 |
| 112357 Normal Lung-F | 0.4 | 0.1 | 0.5 | 112674 OA Synovial Fluid | 0.1 | 0.1 | 0.2 |
| | | | | cells5 | | | |
| 112354 Normal Lung-M | 0.2 | 0.0 | 0.0 | 117100 OA Cartilage | 0.0 | 0.1 | 0.3 |
| | | | | Rep14 | | | |
| 112374 Crohns-F | 0.5 | 0.1 | 0.0 | 112756 OA Bone9 | 0.7 | 1.5 | 1.5 |
| 112389 Match Control | 0.0 | 0.0 | 0.2 | 112757 OA Synovium9 | 100.0 | 100.0 | 100.0 |
| Crohns-F | | | | | | | |
| 112375 Crohns-F | 0.3 | 0.0 | 45.1 | 112758 OA Synovial Fluid | 0.0 | 0.0 | 0.4 |
| | | | | Cells9 | | | |
| 112732 Match Control | 0.1 | 0.1 | 0.1 | 117125 RA Cartilage Rep2 | 0.0 | 0.0 | 0.0 |
| Crohns-F | | | | | | | |
| 112725 Crohns-M | 0.1 | 0.0 | 0.0 | 113492 Bone2 RA | 0.1 | 0.0 | 0.0 |
| 112387 Match Control | 0.1 | 0.0 | 0.0 | 113493 Synovium2 RA | 0.0 | 0.0 | 0.0 |
| Crohns-M | | | | | | | |
| 112378 Crohns-M | 0.1 | 0.1 | 0.4 | 113494 Syn Fluid Cells RA | 0.2 | 0.0 | 0.0 |
| 112390 Match Control | 0.9 | 0.8 | 1.6 | 113499 Cartilage4 RA | 0.0 | 0.0 | 0.0 |
| Crohns-M | | | | | | | |
| 112726 Crohns-M | 0.1 | 0.0 | 0.0 | 113500 Bone4 RA | 0.0 | 0.0 | 0.0 |
| 112731 Match Control | 0.0 | 0.0 | 0.0 | 113501 Synovium4 RA | 0.0 | 0.0 | 0.2 |
| Crohns-M | | | | | | | |
| 112380 Ulcer Col-F | 0.1 | 0.0 | 0.2 | 113502 Syn Fluid Cells4 | 0.0 | 0.0 | 0.0 |
| | | | | RA | | | |
| 112734 Match Control | 3.8 | 4.1 | 6.4 | 113495 Cartilage3 RA | 0.0 | 0.0 | 0.0 |
| Ulcer Col-F | | | | | | | |
| 112384 Ulcer Col-F | 0.9 | 0.5 | 0.7 | 113496 Bone3 RA | 0.0 | 0.1 | 0.0 |
| 112737 Match Control | 0.0 | 0.0 | 0.0 | 113497 Synovium3 RA | 0.0 | 0.0 | 0.0 |
| Ulcer Col-F | | | | | | | |
| 112386 Ulcer Col-F | 0.0 | 0.0 | 0.2 | 113498 Syn Fluid Cells3 | 0.0 | 0.0 | 0.0 |
| | | | | RA | | | |
| 112738 MatchControl | 0.1 | 0.0 | 0.4 | 117106 Normal Cartilage | 0.0 | 0.0 | 0.5 |
| Ulcer Col-F | | | | Rep20 | | | |
| 112381 Ulcer Col-M | 0.0 | 0.0 | 0.0 | 113663 Bone3 Normal | 0.1 | 0.0 | 0.0 |
| 112735 Match Control | 0.1 | 0.3 | 0.0 | 113664 Synovium3 Normal | 0.0 | 0.0 | 0.0 |
| Ulcer Col-M | | | | | | | |
| 112382 Ulcer Col-M | 0.0 | 0.0 | 0.0 | 113665 Syn Fluid Cells3 | 0.0 | 0.1 | 0.0 |
| | | | | Normal | | | |
| 112394 Match Control | 0.1 | 0.0 | 0.0 | 117107 Normal Cartilage | 0.0 | 0.0 | 0.3 |
| Ulcer Col-M | | | | Rep22 | | | |
| 112383 Ulcer Col-M | 0.3 | 0.9 | 1.0 | 113667 Bone4 Normal | 0.0 | 0.0 | 0.3 |
| 112736 Match Control | 0.1 | 0.0 | 0.0 | 113668 Synovium4 Normal | 0.1 | 0.8 | 0.5 |
| Ulcer Col-M | | | | | | | |
| 112423 Psoriasis-F | 0.0 | 0.0 | 0.3 | 113669 Syn Fluid Cells4 | 0.1 | 0.1 | 0.7 |
| | | | | Normal | | | |

TABLE OD
Panel 1.3D
Column A - Rel. Exp.(%) Ag1289, Run 146789972

| Tissue Name | A | Tissue Name | A |
|---|---|---|---|
| Liver adenocarcinoma | 0.1 | Kidney (fetal) | 0.0 |
| Pancreas | 0.2 | Renal ca. 786-0 | 0.0 |
| Pancreatic ca. | 0.0 | Renal ca. A498 | 0.0 |
| CAPAN 2 | | | |
| Adrenal gland | 0.0 | Renal ca. RXF 393 | 0.0 |
| Thyroid | 1.4 | Renal ca. ACHN | 0.0 |
| Salivary gland | 0.2 | Renal ca. UO-31 | 0.0 |
| Pituitary gland | 0.0 | Renal ca. TK-10 | 0.1 |
| Brain (fetal) | 0.0 | Liver | 0.0 |
| Brain (whole) | 0.1 | Liver (fetal) | 0.0 |
| Brain (amygdala) | 0.2 | Liver ca. (hepatoblast) HepG2 | 0.0 |
| Brain (cerebellum) | 0.0 | Lung | 0.0 |
| Brain (hippocampus) | 0.1 | Lung (fetal) | 0.1 |
| Brain | 0.1 | Lung ca. (small cell) LX-1 | 0.0 |
| (substantia nigra) | | | |
| Brain (thalamus) | 0.0 | Lung ca. (small cell) NCI-H69 | 0.1 |
| Cerebral Cortex | 0.4 | Lung ca. (s. cell var.) SHP-77 | 0.2 |
| Spinal cord | 0.1 | Lung ca. (large cell)NCI-H460 | 0.0 |
| glio/astro U87-MG | 0.0 | Lung ca. (non-sm. cell) A549 | 0.0 |
| glio/astro U-118-MG | 0.0 | Lung ca. (non-s. cell) NCI-H23 | 0.0 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.1 | Lung ca. (squam.) NCI-H596 | 0.0 |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA-MB-231 | 0.0 |
| Heart (Fetal) | 0.0 | Breast ca.* (pl.ef) T47D | 0.0 |
| Heart | 0.0 | Breast ca. BT-549 | 0.1 |
| Skeletal muscle (Fetal) | 100.0 | Breast ca. MDA-N | 0.0 |
| Skeletal muscle | 28.7 | Ovary | 0.1 |
| Bone marrow | 0.2 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca. (ascites) SK-OV-3 | 0.1 |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone met) PC-3 | 0.0 |
| Colon ca. HCT-116 | 0.0 | Testis | 0.2 |
| Colon ca. CaCo-2 | 0.0 | Melanoma Hs688(A).T | 0.0 |
| CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.1 | Melanoma* (met) SK-MEL-5 | 0.0 |
| Kidney | 0.0 | Adipose | 0.4 |

TABLE OE
Panel 4D
Column A - Rel. Exp.(%) Ag764, Run 145632998

| Tissue Name | A | Tissue Name | A |
|---|---|---|---|
| Secondary Th1 act | 6.6 | HUVEC IL-1beta | 6.8 |
| Secondary Th2 act | 5.4 | HUVEC IFN gamma | 4.3 |
| Secondary Tr1 act | 4.2 | HUVEC TNF alpha + IFN gamma | 1.3 |
| Secondary Th1 rest | 0.0 | HUVEC TNF alpha + IL4 | 4.4 |
| Secondary Th2 rest | 1.1 | HUVEC IL-11 | 8.3 |
| Secondary Tr1 rest | 0.0 | Lung Microvascular EC none | 43.8 |
| Primary Th1 act | 5.0 | Lung Microvascular EC TNF alpha + IL- | 14.0 |
| | | 1beta | |
| Primary Th2 act | 2.6 | Microvascular Dermal EC none | 100.0 |
| Primary Tr1 act | 4.4 | Microvasular Dermal EC TNF alpha + IL- | 10.4 |
| | | 1beta | |
| Primary Th1 rest | 7.4 | Bronchial epithelium TNF alpha + IL1beta | 0.0 |
| Primary Th2 rest | 3.5 | Small airway epithelium none | 0.0 |
| Primary Tr1 rest | 3.0 | Small airway epithelium TNF alpha + IL- | 0.0 |
| | | 1beta | |
| CD45RA CD4 lymphocyte act | 3.9 | Coronery artery SMC rest | 17.0 |
| CD45RO CD4 lymphocyte act | 2.6 | Coronery artery SMC TNF alpha + IL-1beta | 5.6 |
| CD8 lymphocyte act | 2.5 | Astrocytes rest | 0.7 |
| Secondary CD8 lymphocyte rest | 4.7 | Astrocytes TNF alpha + IL-1beta | 0.0 |
| Secondary CD8 lymphocyte act | 2.3 | KU-812 (Basophil) rest | 3.0 |
| CD4 lymphocyte none | 0.0 | KU-812 (Basophil) PMA/ionomycin | 0.0 |
| 2ry Th1/Th2/Tr1 anti-CD95 | 2.6 | CCD1106 (Keratinocytes) none | 2.5 |
| CH11 | | | |
| LAK cells rest | 1.8 | CCD1106 (Keratinocytes) TNF alpha + IL- | 0.9 |
| | | 1beta | |
| LAK cells IL-2 | 3.3 | Liver cirrhosis | 2.3 |
| LAK cells IL-2 + IL-12 | 3.4 | Lupus kidney | 1.9 |
| LAK cells IL-2 + IFN gamma | 2.6 | NCI-H292 none | 2.1 |
| LAK cells IL-2 + IL-18 | 2.0 | NCI-H292 IL-4 | 3.1 |
| LAK cells PMA/ionomycin | 0.0 | NCI-H292 IL-9 | 3.6 |
| NK Cells IL-2 rest | 3.1 | NCI-H292 IL-13 | 5.3 |
| Two Way MLR 3 day | 0.0 | NCI-H292 IFN gamma | 0.6 |
| Two Way MLR 5 day | 3.3 | HPAEC none | 3.4 |
| Two Way MLR 7 day | 1.2 | HPAEC TNF alpha + IL-1beta | 1.6 |
| PBMC rest | 0.0 | Lung fibroblast none | 0.0 |
| PBMC PWM | 4.5 | Lung fibroblast TNF alpha + IL-1beta | 0.0 |
| PBMC PHA-L | 7.0 | Lung fibroblast IL-4 | 0.0 |
| Ramos (B cell) none | 7.5 | Lung fibroblast IL-9 | 0.0 |
| Ramos (B cell) ionomycin | 8.6 | Lung fibroblast IL-13 | 0.0 |
| B lymphocytes PWM | 2.1 | Lung fibroblast IFN gamma | 0.0 |
| B lymphocytes CD40L and IL-4 | 4.6 | Dermal fibroblast CCD1070 rest | 5.6 |
| EOL-1 dbcAMP | 0.8 | Dermal fibroblast CCD1070 TNF alpha | 19.8 |
| EOL-1 dbcAMP | 0.0 | Dermal fibroblast CCD1070 IL-1beta | 5.7 |
| PMA/ionomycin | | | |
| Dendritic cells none | 0.0 | Dermal fibroblast IFN gamma | 4.7 |
| Dendritic cells LPS | 0.0 | Dermal fibroblast IL-4 | 2.4 |
| Dendritic cells anti-CD40 | 0.0 | IBD Colitis 2 | 0.0 |
| Monocytes rest | 0.0 | IBD Crohn′s | 0.0 |
| Monocytes LPS | 1.0 | Colon | 3.6 |
| Macrophages rest | 0.5 | Lung | 3.0 |
| Macrophages LPS | 0.0 | Thymus | 0.0 |
| HUVEC none | 15.9 | Kidney | 3.8 |
| HUVEC starved | 47.6 | | |

AI_comprehensive panel_v1.0 Summary: Ag1289/Ag764 This gene was moderately expressed in a synovium sample from an osteoarthritis patient. Therefore, therapeutic modulation of this gene, expressed protein and/or use of antibodies or small molecule drugs targeting the gene or gene product is useful in the treatment of osteoarthritis.

The gene variant recognized by probe Ag764 was expressed in a Crohn's disease sample. Therefore, therapeutic modulation of this gene, expressed protein and/or use of antibodies or small molecule drugs targeting the gene or gene product are useful in the treatment of Crohn's disease.

Panel 1.3D Summary: Ag1289 Expression of this gene was highest among normal tissues in skeletal muscle, where it is expressed at roughly 10-fold higher levels than fetal skeletal muscle. Therefore, this gene is useful as a marker to differentiate between adult and fetal skeletal muscle.

This gene was also expressed at low levels in thyroid. Therapeutic modulation of this gene, expressed protein and/or use of antibodies or small molecule drugs targeting the gene or gene product is useful in the treatment of endocrine or metabolically related diseases, such as obesity and diabetes.

Panel 4D Summary: Ag764 This gene was highly expressed in untreated endothelial cells including the microvascular endothelium, human umbilical vein endothelial cells (HUVECS) and lung endothelial cells. This transcript was highly expressed in normal tissue and down regulated in activated endothelium. This gene encodes a protein important for a pathway that is involved in maintaining cellular homeostasis with in a tissue. A protein therapeutic designed with the protein encoded for by this transcript is useful for the reduction or elimination of inflammation in endothelium. Therapeutic modulation of this gene, expressed protein and/or use of antibodies or small molecule drugs targeting the gene or gene product is useful in the treatment of asthma, allergy, psoriasis and arthritis.

P. CG51752-01 and CG51752-02 and CG51752-03: Trypsin Family Serine Protease Tespec PRO-3

Expression of gene CG51752-01, variant CG51752-02 and full-length physical clone CG51752-03 was assessed using the primer-probe sets Ag1541 and Ag346, described in Tables PA and PB. Results of the RTQ-PCR runs are shown in Tables PC and PD. Please not that the primer-probe set Ag1541 is specific for CG51752-01 and CG51752-02 only.

TABLE PA
Probe Name Ag1541

| | | | Start | SEQ ID |
|---|---|---|---|---|
| Primers | Sequences | Length | Position | No |
| Forward | 5′-agaagaacaccccagggatata-3′ | 22 | 238 | 1196 |
| Probe | TET-5′-cctcgttggtgaactacaacctctgg-3′- | 26 | 210 | 1197 |
| | TAMRA | | | |
| Reverse | 5′-cctctagctgggtcactttctc-3′ | 22 | 185 | 1198 |

TABLE PB
Probe Name Ag346

| | | | Start | SEQ ID |
|---|---|---|---|---|
| Primers | Sequences | Length | Position | No |
| Forward | 5′-ctctgggactcccgagactg-3′ | 20 | 105 | 1199 |
| Probe | TET-5′-cccataggtttctgtttgacagaagtcct | 32 | 130 | 1200 |
| | cct-3′-TAMRA | | | |
| Reverse | 5′-aggcccttcaatgcagagaa-3′ | 20 | 163 | 1201 |

TABLE PC
Panel 1.3D
Column A - Rel. Exp.(%) Ag1541, Run 146287841
Column B - Rel. Exp.(%) Ag1541, Run 150033538
Column C - Rel. Exp.(%) Ag346, Run 153910033

| Tissue Name | A | B | C | Tissue Name | A | B | C |
|---|---|---|---|---|---|---|---|
| Liver adenocarcinoma | 0.0 | 0.0 | 0.0 | Kidney (fetal) | 0.5 | 0.6 | 0.0 |
| Pancreas | 0.0 | 0.0 | 1.2 | Renal Ca. 786-0 | 0.0 | 0.0 | 0.0 |
| Pancreatic ca. CAPAN 2 | 0.0 | 0.0 | 1.4 | Renal ca. A498 | 0.0 | 0.0 | 0.0 |
| Adrenal gland | 0.0 | 0.0 | 0.0 | Renal ca. RXF 393 | 0.0 | 0.0 | 0.0 |
| Thyroid | 0.0 | 0.0 | 0.0 | Renal ca. ACHN | 0.0 | 0.0 | 0.0 |
| Salivary gland | 0.0 | 0.0 | 0.0 | Renal ca. UO-31 | 0.0 | 0.0 | 0.0 |
| Pituitary gland | 0.0 | 0.0 | 0.8 | Renal ca. TK-10 | 0.0 | 0.0 | 0.0 |
| Brain (fetal) | 0.5 | 0.4 | 1.2 | Liver | 0.0 | 0.0 | 0.0 |
| Brain (whole) | 1.1 | 1.7 | 6.3 | Liver (fetal) | 0.2 | 0.0 | 0.0 |
| Brain (amygdala) | 0.0 | 1.7 | 1.5 | Liver ca. (hepatoblast) | 0.0 | 0.0 | 0.0 |
| | | | | HepG2 | | | |
| Brain (cerebellum) | 0.6 | 1.9 | 3.4 | Lung | 0.0 | 0.0 | 0.0 |
| Brain (hippocampus) | 3.3 | 3.4 | 6.7 | Lung (fetal) | 0.0 | 0.0 | 0.0 |
| Brain (substantia nigra) | 0.5 | 0.6 | 0.0 | Lung ca. (small cell) LX- | 1.7 | 2.3 | 5.2 |
| | | | | 1 | | | |
| Brain (thalamus) | 1.0 | 1.2 | 0.0 | Lung ca. (small cell) NCI- | 0.0 | 0.0 | 0.0 |
| Cerebral Cortex | 1.6 | 2.6 | 7.7 | Lung ca. (s.cell var.) | 1.3 | 2.5 | 0.0 |
| | | | | SHP-77 | | | |
| Spinal cord | 2.5 | 0.4 | 2.1 | Lung ca. (large cell) NCI- | 0.0 | 0.0 | 0.0 |
| | | | | H460 | | | |
| glio/astro U87-MG | 0.0 | 0.0 | 0.0 | Lung ca. (non-sm. cell) | 0.0 | 0.0 | 1.0 |
| | | | | A549 | | | |
| glio/astro U-118-MG | 0.0 | 0.0 | 0.0 | Lung Ca. (non-s.cell) | 1.2 | 0.4 | 2.6 |
| | | | | NCI-H23 | | | |
| astrocytoma SW1783 | 0.0 | 0.0 | 0.0 | Lung Ca. (non-s.cell) | 0.0 | 0.0 | 0.0 |
| | | | | HOP-62 | | | |
| neuro*; met SK-N-AS | 0.0 | 0.0 | 0.0 | Lung Ca. (non-s.cl) NCI- | 0.0 | 0.0 | 0.9 |
| | | | | H522 | | | |
| astrocytoma SF-539 | 0.0 | 0.0 | 0.0 | Lung ca. (squam.) SW | 0.0 | 0.7 | 0.0 |
| | | | | 900 | | | |
| astrocytoma SNB-75 | 0.7 | 0.0 | 0.0 | Lung Ca. (squam.) NCI- | 0.0 | 1.3 | 0.0 |
| | | | | H596 | | | |
| glioma SNB-19 | 0.0 | 0.0 | 2.7 | Mammary gland | 0.0 | 1.5 | 0.0 |
| glioma U251 | 0.0 | 0.0 | 0.0 | Breast ca.* (pl.ef) MCF-7 | 0.0 | 0.0 | 0.0 |
| glioma SF-295 | 0.0 | 0.8 | 1.0 | Breast ca.* (pl.ef) MDA- | 5.8 | 0.5 | 2.7 |
| | | | | MB-231 | | | |
| Heart (Fetal) | 0.0 | 0.0 | 0.0 | Breast ca.* (pl.ef) T47D | 1.2 | 0.3 | 6.1 |
| Heart | 0.0 | 0.0 | 0.0 | Breast Ca. BT-549 | 0.5 | 0.0 | 2.1 |
| Skeletal muscle (Fetal) | 0.6 | 1.6 | 1.4 | Breast Ca. MDA-N | 0.0 | 0.0 | 0.1 |
| Skeletal muscle | 0.0 | 0.0 | 0.0 | Ovary | 0.0 | 0.0 | 0.0 |
| Bone marrow | 0.0 | 0.0 | 2.1 | Ovarian ca. OVCAR-3 | 0.0 | 0.0 | 1.7 |
| Thymus | 0.0 | 0.0 | 0.0 | Ovarian ca. OVCAR-4 | 0.0 | 0.0 | 0.0 |
| Spleen | 0.0 | 0.0 | 1.6 | Ovarian ca. OVCAR-5 | 3.6 | 0.7 | 3.2 |
| Lymph node | 0.0 | 0.0 | 0.0 | Ovarian ca. OVCAR-8 | 0.0 | 0.0 | 0.0 |
| Colorectal | 0.0 | 0.6 | 2.2 | Ovarian ca. IGROV-1 | 0.0 | 0.0 | 0.0 |
| Stomach | 1.9 | 0.0 | 0.0 | Ovarian ca. (ascites) SK- | 0.0 | 0.0 | 0.0 |
| | | | | OV-3 | | | |
| Small intestine | 0.0 | 1.0 | 0.0 | Uterus | 0.0 | 0.0 | 1.6 |
| Colon ca. SW480 | 0.0 | 0.0 | 0.0 | Placenta | 0.0 | 0.0 | 0.0 |
| Colon ca.* SW620 (SW480 | 0.0 | 0.0 | 0.0 | Prostate | 0.0 | 0.7 | 1.5 |
| met) | | | | | | | |
| Colon ca. HT29 | 0.0 | 0.0 | 0.0 | Prostate ca.* (bone met) | 0.0 | 0.0 | 0.0 |
| | | | | PC-3 | | | |
| Colon ca. HCT-116 | 0.6 | 0.4 | 0.0 | Testis | 100.0 | 100.0 | 100.0 |
| Colon ca. CaCo-2 | 1.5 | 0.0 | 0.0 | Melanoma Hs688(A).T | 0.0 | 0.0 | 0.0 |
| CC Well to Mod Duff | 0.0 | 0.0 | 0.0 | Melanoma* (met) | 0.0 | 0.0 | 0.0 |
| (ODO3866) | | | | Hs688(B).T | | | |
| Colon ca. HCC-2998 | 0.0 | 0.0 | 0.0 | Melanoma UACC-62 | 0.0 | 0.0 | 0.0 |
| Gastric ca. (liver met) NCI- | 1.2 | 0.0 | 0.0 | Melanoma M14 | 0.0 | 0.0 | 0.0 |
| N87 | | | | | | | |
| Bladder | 0.0 | 0.0 | 0.0 | Melanoma LOX IMVI | 0.0 | 0.0 | 0.0 |
| Trachea | 0.0 | 0.4 | 0.0 | Melanoma* (met) SK- | 0.0 | 0.0 | 0.0 |
| | | | | MEL-5 | | | |
| Kidney | 0.8 | 1.2 | 2.6 | Adipose | 0.5 | 0.0 | 0.0 |

TABLE PD
Panel 2D
Column A - Rel. Exp.(%) Ag1541, Run 149802457
Column B - Rel. Exp.(%) Ag1541, Run 150033539
Column C - Rel. Exp.(%) Ag1541, Run 162624512

| Tissue Name | A | B | C | Tissue Name | A | B | C |
|---|---|---|---|---|---|---|---|
| Normal Colon | 5.4 | 2.4 | 0.0 | Kidney Margin 8120608 | 0.0 | 0.0 | 0.0 |
| CC Well to Mod Diff | 7.3 | 0.0 | 5.1 | Kidney Cancer 8120613 | 0.0 | 0.0 | 0.0 |
| (ODO3866) | | | | | | | |
| CC Margin (ODO3866) | 5.8 | 1.5 | 0.0 | Kidney Margin 8120614 | 20.6 | 22.8 | 16.6 |
| CC Gr.2 rectosigmoid | 3.4 | 0.0 | 0.0 | Kidney Cancer 9010320 | 0.0 | 0.0 | 0.0 |
| (ODO3868) | | | | | | | |
| CC Margin (ODO3868) | 0.0 | 0.0 | 0.0 | Kidney Margin 9010321 | 3.4 | 26.4 | 14.8 |
| CC Mod Diff (ODO3920) | 11.0 | 1.4 | 2.0 | Normal Uterus | 0.0 | 0.0 | 0.0 |
| CC Margin (ODO3920) | 0.0 | 0.0 | 3.4 | Uterine Cancer 064011 | 14.9 | 0.0 | 4.4 |
| CC Gr.2 ascend colon | 6.2 | 2.5 | 6.1 | Normal Thyroid | 0.0 | 0.0 | 0.0 |
| (ODO3921) | | | | | | | |
| CC Margin (ODO3921) | 10.2 | 0.0 | 6.0 | Thyroid Cancer | 0.0 | 0.0 | 0.0 |
| CC from Partial | 3.6 | 0.0 | 4.5 | Thyroid Cancer | 0.0 | 0.0 | 0.0 |
| Hepatectomy (ODO4309) | | | | A302152 | | | |
| Mets | | | | | | | |
| Liver Margin (ODO4309) | 0.0 | 2.4 | 4.7 | Thyroid Margin | 0.0 | 0.0 | 5.1 |
| | | | | A302153 | | | |
| Colon mets to lung | 7.2 | 4.4 | 7.6 | Normal Breast | 5.2 | 3.5 | 7.0 |
| (ODO4451-01) | | | | | | | |
| Lung Margin (ODO4451-02) | 0.0 | 0.0 | 0.0 | Breast Cancer | 0.0 | 0.0 | 2.4 |
| Normal Prostate 6546-1 | 4.8 | 2.9 | 22.1 | Breast Cancer | 0.0 | 0.0 | 0.0 |
| | | | | (ODO4590-01) | | | |
| Prostate Cancer (ODO4410) | 3.5 | 0.0 | 0.0 | Breast Cancer Mets | 0.0 | 0.0 | 0.0 |
| | | | | (ODO4590-03) | | | |
| Prostate Margin (ODO4410) | 3.4 | 0.0 | 0.0 | Breast Cancer | 0.0 | 0.0 | 0.0 |
| | | | | Metastasis | | | |
| Prostate Cancer (ODO4720- | 9.0 | 8.5 | 43.8 | Breast Cancer | 0.0 | 2.5 | 0.0 |
| 01) | | | | | | | |
| Prostate Margin (ODO4720- | 0.0 | 0.0 | 0.0 | Breast Cancer | 52.5 | 27.5 | 55.5 |
| 02) | | | | | | | |
| Normal Lung | 17.7 | 6.5 | 0.0 | Breast Cancer 9100266 | 6.2 | 0.0 | 0.0 |
| Lung Met to Muscle | 0.0 | 2.3 | 9.9 | Breast Margin 9100265 | 0.0 | 0.0 | 1.9 |
| (ODO4286) | | | | | | | |
| Muscle Margin (ODO4286) | 0.0 | 0.0 | 0.0 | Breast Cancer A209073 | 1.5 | 2.5 | 3.5 |
| Lung Malignant Cancer | 6.5 | 5.7 | 4.9 | Breast Margin A209073 | 24.3 | 26.2 | 61.1 |
| (ODO3126) | | | | | | | |
| Lung Margin (ODO3126) | 0.0 | 0.0 | 0.0 | Normal Liver | 10.5 | 2.7 | 4.2 |
| Lung Cancer (ODO4404) | 0.0 | 0.0 | 0.0 | Liver Cancer | 5.9 | 1.7 | 3.2 |
| Lung Margin (ODO4404) | 0.0 | 0.0 | 3.4 | Liver Cancer 1025 | 21.6 | 11.0 | 13.6 |
| Lung Cancer (ODO4565) | 0.0 | 0.0 | 0.0 | Liver Cancer 1026 | 0.0 | 0.0 | 0.0 |
| Lung Margin (ODO4565) | 0.0 | 0.0 | 0.0 | Liver Cancer 6004-T | 3.3 | 13.5 | 2.7 |
| Lung Cancer (OD04237-01) | 0.0 | 0.0 | 0.0 | Liver Tissue 6004-N | 3.2 | 1.4 | 0.0 |
| Lung Margin (ODO4237-02) | 0.0 | 0.0 | 0.0 | Liver Cancer 6005-T | 0.0 | 0.0 | 0.0 |
| Ocular Mel Met to Liver | 4.3 | 0.0 | 3.2 | Liver Tissue 6005-N | 0.0 | 0.0 | 0.0 |
| (ODO4310) | | | | | | | |
| Liver Margin (ODO4310) | 0.0 | 0.0 | 0.0 | Normal Bladder | 0.0 | 0.0 | 5.9 |
| Melanoma Metastasis | 0.0 | 0.0 | 0.0 | Bladder Cancer | 0.0 | 0.0 | 0.0 |
| Lung Margin (ODO4321) | 0.0 | 0.0 | 0.0 | Bladder Cancer | 4.6 | 2.3 | 7.5 |
| Normal Kidney | 28.1 | 39.2 | 54.0 | Bladder Cancer | 17.9 | 11.4 | 17.8 |
| | | | | (ODO4718-01) | | | |
| Kidney Ca, Nuclear grade 2 | 0.0 | 3.0 | 0.0 | Bladder Normal | 0.0 | 0.0 | 0.0 |
| (ODO4338) | | | | Adjacent (ODO4718-03) | | | |
| Kidney Margin (ODO4338) | 22.7 | 31.6 | 24.1 | Normal Ovary | 0.0 | 0.0 | 0.0 |
| Kidney Ca Nuclear grade 1/2 | 0.0 | 3.1 | 2.9 | Ovarian Cancer | 1.7 | 4.8 | 8.2 |
| (ODO4339) | | | | | | | |
| Kidney Margin (ODO4339) | 97.3 | 100.0 | 100.0 | Ovarian Cancer | 0.0 | 2.1 | 0.0 |
| | | | | (ODO4768-07) | | | |
| Kidney Ca, Clear cell type | 0.0 | 0.0 | 0.0 | Ovary Margin | 0.0 | 0.0 | 0.0 |
| (ODO4340) | | | | (ODO4768-08) | | | |
| Kidney Margin (ODO4340) | 100.0 | 34.4 | 33.4 | Normal Stomach | 3.3 | 2.9 | 0.0 |
| Kidney Ca, Nuclear grade 3 | 2.0 | 4.9 | 3.1 | Gastric Cancer 9060358 | 0.0 | 0.0 | 0.0 |
| (ODO4348) | | | | | | | |
| Kidney Margin (ODO4348) | 30.1 | 19.9 | 20.9 | Stomach Margin | 0.0 | 0.0 | 0.0 |
| | | | | 9060359 | | | |
| Kidney Cancer (ODO4622- | 0.0 | 2.4 | 0.0 | Gastric Cancer 9060395 | 0.0 | 0.0 | 0.0 |
| 01) | | | | | | | |
| Kidney Margin (ODO4622- | 8.4 | 7.2 | 13.9 | Stomach Margin | 0.0 | 0.0 | 0.0 |
| 03) | | | | 9060394 | | | |
| Kidney Cancer (ODO4450- | 0.0 | 0.0 | 0.0 | Gastric Cancer 9060397 | 0.0 | 0.0 | 0.0 |
| 01) | | | | | | | |
| Kidney Margin (ODO4450- | 47.3 | 12.9 | 30.6 | Stomach Margin | 0.0 | 0.0 | 0.0 |
| 03) | | | | 9060396 | | | |
| Kidney Cancer 8120607 | 0.0 | 0.0 | 0.0 | Gastric Cancer 064005 | 6.3 | 3.8 | 2.4 |

Panel 1.3D Summary: Ag346/Ag1541 The expression of this gene was only detected in the testis (CT=31). Gene or protein expression levels of this gene are useful as a marker for the detection of testis tissue. Therapeutic modulation of the activity of this gene or its protein product is useful for the treatment of male infertility.

Panel 2D Summary: Ag1541 Expression of this gene was highest in normal kidney (CT=31-32). This gene was significantly overexpressed in 8/9 normal kidney samples when compared to the adjacent tumor samples. Therefore, the gene or protein expression levels are useful as a marker to distinguish normal kidney from kidney tumors. Therapeutic modulation of the activity of this gene or its protein product using protein, antibody or small molecule drugs is useful in the treatment of kidney cancer.

Q. CG51914-02: Ephrin Type-A Receptor 7 Precursor

Expression of gene CG51914-02 was assessed using the primer-probe set Ag612, described in Table QA. Results of the RTQ-PCR runs are shown in Tables QB, QC and QD.

TABLE QA
Probe Name Ag612

| | | | Start | SEQ ID |
|---|---|---|---|---|
| Primers | Sequences | Length | Position | No |
| Forward | 5′-gccgctcccgagacactt-3′ | 18 | 2558 | 1202 |
| Probe | TET-5′-ccacttcagctctgccagtgacgtg-3′- | 25 | 2584 | 1203 |
| | TAMRA | | | |
| Reverse | 5′-cccacatgatgatgccgaa-3′ | 19 | 2615 | 1204 |

TABLE QB
CNS_neurodegeneration_v1.0
Column A - Rel. Exp.(%) Ag612, Run 309606071

| Tissue Name | A | Tissue Name | A |
|---|---|---|---|
| AD 1 Hippo | 46.0 | AH3 4624 | 31.0 |
| AD 2 Hippo | 37.1 | AH3 4640 | 100.0 |
| AD 3 Hippo | 9.5 | AD 1 Occipital Ctx | 11.0 |
| AD 4 Hippo | 33.9 | AD 2 Occipital Ctx (Missing) | 6.8 |
| AD 5 Hippo | 59.5 | AD 3 Occipital Ctx | 8.3 |
| AD 6 Hippo | 41.8 | AD 4 Occipital Ctx | 35.6 |
| Control 2 Hippo | 40.9 | AD 5 Occipital Ctx | 43.5 |
| Control 4 Hippo | 21.6 | AD 5 Occipital Ctx | 21.5 |
| Control (Path) 3 Hippo | 15.9 | Control 1 Occipital Ctx | 8.2 |
| AD 1 Temporal Ctx | 26.1 | Control 2 Occipital Ctx | 36.6 |
| AD 2 Temporal Ctx | 28.9 | Control 3 Occipital Ctx | 29.5 |
| AD 3 Temporal Ctx | 12.3 | Control 4 Occipital Ctx | 20.3 |
| AD 4 Temporal Ctx | 62.9 | Control (Path) 1 Occipital Ctx | 81.2 |
| AD 5 Inf Temporal Ctx | 57.4 | Control (Path) 2 Occipital Ctx | 37.4 |
| AD 5 Sup Temporal Ctx | 33.7 | Control (Path) 3 Occipital Ctx | 17.8 |
| AD 6 Inf Temporal Ctx | 50.7 | Control (Path) 4 Occipital Ctx | 29.9 |
| AD 6 Sup Temporal Ctx | 68.8 | Control 1 Parietal Ctx | 37.6 |
| Control 1 Temporal Ctx | 9.7 | Control 2 Parietal Ctx | 41.5 |
| Control 2 Temporal Ctx | 54.7 | Control 3 Parietal Ctx | 54.0 |
| Control 3 Temporal Ctx | 40.3 | Control (Path) 1 Parietal Ctx | 99.3 |
| Control 3 Temporal Ctx | 43.2 | Control (Path) 2 Parietal Ctx | 52.9 |
| AH3 3975 | 97.3 | Control (Path) 3 Parietal Ctx | 11.6 |
| AH3 3954 | 86.5 | Control (Path) 4 Parietal Ctx | 56.6 |

TABLE QC
Panel 1.1
Column A - Rel. Exp.(%) Ag612, Run 109649311

| Tissue Name | A | Tissue Name | A |
|---|---|---|---|
| Adrenal gland | 0.7 | Renal ca. UO-31 | 0.0 |
| Bladder | 25.0 | Renal ca. RXF 393 | 1.0 |
| Brain (amygdala) | 12.9 | Liver | 0.0 |
| Brain (cerebellum) | 7.7 | Liver (fetal) | 0.0 |
| Brain (hippocampus) | 39.8 | Liver ca. (hepatoblast) HepG2 | 0.3 |
| Brain (substantia nigra) | 50.0 | Lung | 0.0 |
| Brain (thalamus) | 20.9 | Lung (fetal) | 0.0 |
| Cerebral Cortex | 65.5 | Lung ca. (non-s. cell) HOP-62 | 0.3 |
| Brain (fetal) | 44.1 | Lung ca. (large cell)NCI-H460 | 1.9 |
| Brain (whole) | 36.3 | Lung ca. (non-s. cell) NCI-H23 | 18.3 |
| glio/astro U-118-MG | 0.0 | Lung ca. (non-s. cl) NCI-H522 | 100.0 |
| astrocytoma SF-539 | 0.0 | Lung ca. (non-sm. cell) A549 | 10.5 |
| astrocytoma SNB-75 | 0.0 | Lung ca. (s. cell var.) SHP-77 | 3.4 |
| astrocytoma SW1783 | 0.0 | Lung ca. (small cell) LX-1 | 1.5 |
| glioma U251 | 0.0 | Lung ca. (small cell) NCI-H69 | 3.5 |
| glioma SF-295 | 0.0 | Lung ca. (squam.) SW 900 | 24.1 |
| glioma SNB-19 | 0.0 | Lung ca. (squam.) NCI-H596 | 0.7 |
| glio/astro U87-MG | 0.0 | Lymph node | 0.3 |
| neuro*; met SK-N-AS | 0.0 | Spleen | 0.0 |
| Mammary gland | 4.3 | Thymus | 0.0 |
| Breast ca. BT-549 | 0.0 | Ovary | 0.1 |
| Breast ca. MDA-N | 0.1 | Ovarian ca. IGROV-1 | 1.8 |
| Breast ca.* (pl.ef) T47D | 18.6 | Ovarian ca. OVCAR-3 | 4.0 |
| Breast ca.* (pl.ef) | 14.9 | Ovarian ca. OVCAR-4 | 0.0 |
| MCF-7 | | | |
| Breast ca.* (pl.ef) | 0.1 | Ovarian ca. OVCAR-5 | 36.6 |
| MDA-MB-231 | | | |
| Small intestine | 14.8 | Ovarian ca. OVCAR-8 | 11.3 |
| Colorectal | 44.1 | Ovarian ca. (ascites) SK-OV-3 | 22.5 |
| Colon ca. HT29 | 0.7 | Pancreas | 64.2 |
| Colon ca. CaCo-2 | 3.4 | Pancreatic ca. CAPAN 2 | 1.1 |
| Colon ca. HCT-15 | 34.2 | Pituitary gland | 33.4 |
| Colon ca. HCT-116 | 6.1 | Placenta | 0.0 |
| Colon ca. HCC-2998 | 24.5 | Prostate | 3.3 |
| Colon ca. SW480 | 3.7 | Prostate ca.* (bone met) PC-3 | 4.2 |
| Colon ca.* SW620 | 4.9 | Salivary gland | 15.7 |
| (SW480 met) | | | |
| Stomach | 10.6 | Trachea | 1.6 |
| Gastric ca. (liver met) | 33.7 | Spinal cord | 5.8 |
| NCI-N87 | | | |
| Heart | 1.6 | Testis | 65.5 |
| Skeletal muscle (Fetal) | 0.0 | Thyroid | 0.0 |
| Skeletal muscle | 0.0 | Uterus | 0.0 |
| Endothelial cells | 0.0 | Melanoma M14 | 0.0 |
| Heart (Fetal) | 0.0 | Melanoma LOX IMVI | 0.0 |
| Kidney | 1.0 | Melanoma UACC-62 | 0.0 |
| Kidney (fetal) | 0.0 | Melanoma SK-MEL-28 | 0.0 |
| Renal ca. 786-0 | 0.0 | Melanoma* (met) SK-MEL-5 | 0.7 |
| Renal ca. A498 | 0.3 | Melanoma Hs688(A).T | 0.0 |
| Renal ca. ACHN | 7.3 | Melanoma* (met) Hs688(B).T | 0.0 |
| Renal ca. TK-10 | 42.6 | | |

TABLE QD
Panel 4D
Column A - Rel. Exp.(%) Ag612, Run 145645058

| Tissue Name | A | Tissue Name | A |
|---|---|---|---|
| Secondary Th1 act | 0.0 | HUVEC IL-1beta | 0.0 |
| Secondary Th2 act | 12.2 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 0.0 | Microvascular Dermal EC none | 0.0 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL- | 4.6 |
| | | 1beta | |
| CD45RA CD4 lymphocyte act | 0.0 | Coronery artery SMC rest | 0.0 |
| CD45RO CD4 lymphocyte act | 4.6 | Coronery artery SMC TNF alpha + IL-1beta | 0.0 |
| CD8 lymphocyte act | 5.3 | Astrocytes rest | 0.0 |
| Secondary CD8 lymphocyte rest | 5.3 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 9.7 | CCD1106 (Keratinocytes) TNF alpha + IL- | 1.0 |
| | | 1beta | |
| LAK cells IL-2 | 0.0 | Liver cirrhosis | 61.6 |
| LAK cells IL-2 + IL-12 | 0.0 | Lupus kidney | 0.0 |
| LAK cells IL-2 + IFN gamma | 5.1 | NCI-H292 none | 32.5 |
| LAK cells IL-2 + IL-18 | 0.0 | NCI-H292 IL-4 | 46.7 |
| LAK cells PMA/ionomycin | 0.0 | NCI-H292 IL-9 | 58.6 |
| NK Cells IL-2 rest | 0.0 | NCI-H292 IL-13 | 89.5 |
| Two Way MLR 3 day | 0.0 | NCI-H292 IFN gamma | 100.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL-1beta | 0.0 |
| PBMC PHA-L | 8.9 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 9.0 |
| B lymphocytes PWM | 8.5 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1beta | 0.0 |
| PMA/ionomycin | | | |
| Dendritic cells none | 5.3 | Dermal fibroblast IFN gamma | 0.0 |
| Dendritic cells LPS | 4.5 | Dermal fibroblast IL-4 | 0.0 |
| Dendritic cells anti-CD40 | 0.0 | IBD Colitis 2 | 10.7 |
| Monocytes rest | 0.0 | IBD Crohn's | 0.0 |
| Monocytes LPS | 1.2 | Colon | 51.8 |
| Macrophages rest | 0.0 | Lung | 24.7 |
| Macrophages LPS | 0.0 | Thymus | 4.1 |
| HUVEC none | 0.0 | Kidney | 4.6 |
| HUVEC starved | 0.0 | | |

CNS_neurodegeneration_v1.0 Summary: Ag612 This gene was found to be down-regulated in the temporal cortex of Alzheimer's disease patients. Therefore, up-regulation of this gene, encoded protein, and/or use of agonists for this receptor is useful in reversing the dementia/memory loss associated with this disease and neuronal death.

Panel 1.1 Summary: Ag612 Highest expression of this gene was detected in a lung cancer NCI-H522 cell line (CT=24). High expression of this gene was also seen in cluster of lung cancer, colon cancer, renal cancer, a liver cancer, two breast cancer and a melanoma cell lines. Levels of expression of this gene are useful as diagnostic markers and modulation of this gene, encoded protein and/or use of antibodies or small molecule drug targeting this gene or gene product is useful in the treatments of this cancers.

In addition, high expression of this gene was seen in all the regions of the central nervous system (CNS) examined including, amygdala, hippocampus, substantia nigra, thalamus, cerebellum, cerebral cortex, and spinal cord. This gene, encoded protein and/or use of antibodies or small molecule drug targeting this gene or gene product is useful in the treatment of CNS disorders such as Alzheimer's disease, Parkinson's disease, epilepsy, multiple sclerosis, schizophrenia and depression and therapeutic modulation of this gene product may be useful in the treatment of these disorders.

Among tissues with metabolic or endocrine function, this gene was expressed at high to moderate levels in pancreas, adrenal gland, pituitary gland, and the gastrointestinal tract. Modulation of this gene is useful in the treatment of endocrine/metabolically related diseases, such as obesity and diabetes

Panel 4D Summary: Ag612 Highest expression of this gene was detected in IFN gamma treated NCI-H292 cells (CT=33). Moderate to low expression of this gene was also seen in cytokine treated and untreated NCI-H292 cells, liver cirrhosis and colon tissue samples. Modulation of this gene, encoded protein and/or use of antibodies or small molecule drug targeting this gene is useful for the treatment of chronic obstructive pulmonary disease, asthma, allergy, and emphysema, liver cirrhosis, autoimmune and inflammatory disease affecting colon including Crohn's disease and ulcerative colitis.

R. CG51965-01: Protocadherin Flamingo 2 Like

Expression of gene CG51965-01 was assessed using the primer-probe sets Ag1989 and Ag1990, described in Tables RA and RB. Results of the RTQ-PCR runs are shown in Tables RC, RD, RE and RF.

TABLE RA
Probe Name Ag1989

| | | | Start | SEQ ID |
|---|---|---|---|---|
| Primers | Sequences | Length | Position | No |
| Forward | 5′-cctagagatcctcatcctcgat-3′ | 22 | 2631 | 1205 |
| Probe | TET-5′-aatgacaatgcaccccagttcctgt-3′- | 25 | 2656 | 1206 |
| | TAMRA | | | |
| Reverse | 5′-aaagatggaaccctggtagaaa-3′ | 22 | 2685 | 1207 |

TABLE RB
Probe Name Ag1990

| | | | Start | SEQ ID |
|---|---|---|---|---|
| Primers | Sequences | Length | Position | No |
| Forward | 5′-cctagagatcctcatcctcgat-3′ | 22 | 2631 | 1208 |
| Probe | TET-5′-aatgacaatgcaccccagttcctgt-3′- | 25 | 2656 | 1209 |
| | TAMRA | | | |
| Reverse | 5′-aaagatggaaccctggtagaaa-3′ | 22 | 2685 | 1210 |

TABLE RC
AI_comprehensive panel_v1.0
Column A - Rel. Exp.(%) Ag1989, Run 248122025
Column B - Rel. Exp.(%) Ag1990, Run 228059649

| Tissue Name | A | B | Tissue Name | A | B |
|---|---|---|---|---|---|
| 110967 COPD-F | 4.9 | 5.3 | 112427 Match Control Psoriasis-F | 23.0 | 25.5 |
| 110980 COPD-F | 3.8 | 5.7 | 112418 Psoriasis-M | 2.0 | 3.8 |
| 110968 COPD-M | 2.2 | 2.6 | 112723 Match Control Psoriasis- | 1.1 | 1.4 |
| | | | M | | |
| 110977 COPD-M | 9.4 | 17.9 | 112419 Psonasis-M | 4.8 | 5.1 |
| 110989 Emphysema-F | 19.5 | 24 | 1112424 Match Control Psoriasis- | 3.4 | 6.0 |
| | | | M | | |
| 110992 Emphysema-F | 35.4 | 42.3 | 112420 Psoriasis-M | 31.9 | 30.1 |
| 110993 Emphysema-F | 6.3 | 6.7 | 112425 Match Control Psoriasis- | 31.0 | 31.2 |
| | | | M | | |
| 110994 Emphysema-F | 4.1 | 6.3 | 104689 (MF) OA Bone-Backus | 12.7 | 22.1 |
| 110995 Emphysema-F | 54.7 | 59.9 | 104690 (MF) Adj “Normal” Bone- | 7.7 | 9.7 |
| | | | Backus | | |
| 110996 Emphysema-F | 18.6 | 21.5 | 104691 (MF) OA Synovium- | 3.7 | 6.8 |
| | | | Backus | | |
| 110997 Asthma-M | 14.1 | 19.5 | 104692 (BA) OA Cartilage- | 29.9 | 27.7 |
| | | | Backus | | |
| 111001 Asthma-F | 17.4 | 23.3 | 104694 (BA) OA Bone-Backus | 11.3 | 14.5 |
| 111002 Asthma-F | 32.1 | 31.6 | 104695 (BA) Adj “Normal” Bone- | 14.0 | 10.8 |
| | | | Backus | | |
| 111003 Atopic Asthma-F | 24.0 | 28.1 | 104696 (BA) OA Synovium- | 10.5 | 6.2 |
| | | | Backus | | |
| 111004 Atopic Asthma-F | 79.0 | 73.7 | 104700 (SS) OA Bone-Backus | 7.9 | 11.4 |
| 111005 Atopic Asthma-F | 34.6 | 42.0 | 104701 (SS) Adj “Normal” Bone- | 14.3 | 17.8 |
| | | | Backus | | |
| 111006 Atopic Asthma-F | 4.6 | 10.9 | 104702 (SS) OA Synovium- | 14.8 | 19.1 |
| | | | Backus | | |
| 111417 Allergy-M | 27.9 | 39.0 | 117093 OA Cartilage Rep7 | 29.1 | 28.1 |
| 112347 Allergy-M | 0.5 | 1.4 | 112672 OA Bone5 | 12.3 | 12.1 |
| 112349 Normal Lung-F | 0.6 | 0.4 | 112673 OA Synovium5 | 4.3 | 6.9 |
| 112357 Normal Lung-F | 2.5 | 5.7 | 112674 OA Synovial Fluid cells | 3.1 | 5.4 |
| 112354 Normal Lung-M | 2.4 | 3.1 | 117100 OA Cartilage Rep14 | 1.0 | 4.3 |
| 112374 Crohns-F | 6.0 | 6.3 | 112756 OA Bone9 | 100.0 | 100.0 |
| 112389 Match Control | 40.6 | 41.8 | 112757 OA Synovium9 | 0.4 | 0.0 |
| Crohns-F | | | | | |
| 112375 Crohns-F | 5.9 | 5.6 | 112758 OA Synovial Fluid Cells9 | 2.7 | 6.1 |
| 112732 Match Control | 23.2 | 20.9 | 117125 RA Cartilage Rep2 | 5.3 | 5.6 |
| Crohns-F | | | | | |
| 112725 Crobns-M | 1.1 | 1.7 | 113492 Bone2 RA | 53.2 | 64.6 |
| 112387 Match Control | 10.2 | 17.2 | 113493 Synovium2 RA | 28.9 | 22.4 |
| Crohns-M | | | | | |
| 112378 Crohns-M | 10.2 | 1.72 | 113494 Syn Fluid Cells RA | 29.7 | 45.4 |
| 112390 Match Control | 51.4 | 58.6 | 113499 Cartilage4 RA | 32.5 | 37.4 |
| Crohns-M | | | | | |
| 112726 Crohns-M | 19.5 | 22.4 | 113500 Bone4 RA | 35.6 | 48.3 |
| 112731 Match Control | 14.1 | 21.5 | 113501 Synovium4 RA | 21.6 | 27.7 |
| Crohns-M | | | | | |
| 112380 Ulcer Col-F | 21.2 | 27.7 | 113502 Syn Fluid Cells4 RA | 13.3 | 15.2 |
| Col-F | | | | | |
| 112384 Ulcer Col-F | 38.7 | 35.4 | 113496 Bone3 RA | 35.4 | 52.9 |
| 112737 Match Control Ulcer | 3.1 | 11.7 | 113497 Synovium3 RA | 16.4 | 25.7 |
| Col-F | | | | | |
| 112386 Ulcer Col-F | 4.9 | 6.9 | 113498 Syn Fluid Cells3 RA | 41.5 | 55.5 |
| 112738 Match Control Ulcer | 4.2 | 6.6 | 117106 Normal Cartilage Rep20 | 9.9 | 14.2 |
| Col-F | | | | | |
| 112381 Ulcer Col-M | 1.1 | 1.0 | 113663 Bone3 Normal | 1.2 | 0.9 |
| 112735 Match Control Ulcer | 9.9 | 11.3 | 113664 Synovium3 Normal | 0.2 | 0.5 |
| Col-M | | | | | |
| 112382 UlcerCol-M | 21.5 | 27.5 | 113665 SynFluid Cells3 Normal | 0.6 | 0.6 |
| 112394 Match Control Ulcer | 1.2 | 1.6 | 117107 Normal Cartilage Rep22 | 2.7 | 5.6 |
| Col-M | | | | | |
| 112383 Ulcer Col-M | 34.4 | 39.5 | 113667 Bone4 Normal | 10.4 | 9.9 |
| 112736 Match Control Ulcer | 16.2 | 17.8 | 113668 Synovium4 Normal | 5.2 | 9.7 |
| Col-M | | | | | |
| 112423 Psoriasis-F | 1.9 | 3.6 | 113669 Syn Fluid Cells4 Normal | 12.3 | 18.2 |

TABLE RD
Panel 1.3D
Column A - Rel. Exp.(%) Ag1989, Run 147796849
Column B - Rel. Exp.(%) Ag1989, Run 153940861
Column C - Rel. Exp.(%) Ag1990, Run 147797262

| Tissue Name | A | B | C | Tissue Name | A | B | C |
|---|---|---|---|---|---|---|---|
| Liver adenocarcinoma | 37.6 | 54.7 | 37.4 | Kidney (fetal) | 7.2 | 14.3 | 7.6 |
| Pancreas | 0.1 | 1.1 | 0.1 | Renal Ca. 786-0 | 30.4 | 48.0 | 32.8 |
| Pancreatic ca. CAPAN 2 | 8.4 | 15.0 | 10.0 | Renal ca. A498 | 52.9 | 65.5 | 65.5 |
| Adrenal gland | 0.7 | 0.7 | 0.4 | Renal ca. RXF 393 | 29.7 | 34.9 | 34.9 |
| Thyroid | 7.3 | 9.5 | 6.9 | Renal ca. ACHN | 89.5 | 49.0 | 65.5 |
| Salivary gland | 4.0 | 8.2 | 6.4 | Renal ca. UO-31 | 18.9 | 17.7 | 18.4 |
| Pituitary gland | 3.1 | 4.2 | 4.2 | Renal ca. TK-10 | 18.3 | 27.0 | 14.4 |
| Brain (fetal) | 0.4 | 0.5 | 0.2 | Liver | 0.0 | 0.0 | 0.0 |
| Brain (whole) | 0.6 | 0.3 | 0.1 | Liver (fetal) | 0.1 | 0.8 | 0.1 |
| Brain (amygdala) | 0.0 | 0.5 | 0.0 | Liver ca. (hepatoblast) | 0.0 | 0.2 | 0.1 |
| | | | | HepG2 | | | |
| Brain (cerebellum) | 0.0 | 0.0 | 0.0 | Lung | 11.0 | 25.2 | 9.3 |
| Brain (hippocampus) | 0.9 | 1.7 | 0.4 | Lung (fetal) | 14.7 | 12.1 | 13.9 |
| Brain (substantia nigra) | 0.0 | 0.0 | 0.0 | Lung ca. (small cell) LX- | 4.0 | 4.8 | 6.5 |
| | | | | 1 | | | |
| Brain (thalamus) | 0.1 | 0.0 | 0.5 | Lung ca. (small cell) NCI- | 3.4 | 5.3 | 3.1 |
| Cerebral Cortex | 0.1 | 0.0 | 0.4 | Lung ca. (s.cell var.) | 0.8 | 0.5 | 1.1 |
| | | | | SHP-77 | | | |
| Spinal cord | 0.1 | 0.1 | 0.1 | Lung ca. (large cell) NCI- | 8.1 | 8.1 | 11.2 |
| | | | | H460 | | | |
| glio/astro U87-MG | 14.4 | 16.6 | 19.6 | Lung ca. (non-sm. cell) | 13.5 | 17.6 | 13.9 |
| | | | | A549 | | | |
| glio/astro U-118-MG | 5.8 | 5.1 | 7.4 | Lung Ca. (non-s.cell) | 29.3 | 49.7 | 27.2 |
| | | | | NCI-H23 | | | |
| astrocytoma SW1783 | 12.2 | 14.5 | 19.5 | Lung Ca. (non-s.cell) | 33.4 | 46.7 | 39.8 |
| | | | | HOP-62 | | | |
| neuro*; met SK-N-AS | 0.0 | 0.0 | 0.0 | Lung Ca. (non-s.cl) NCI- | 18.6 | 33.2 | 22.8 |
| | | | | H522 | | | |
| astrocytoma SF-539 | 20.4 | 31.4 | 15.4 | Lung ca. (squam.) SW | 18.6 | 16.7 | 22.4 |
| | | | | 900 | | | |
| astrocytoma SNB-75 | 40.6 | 26.4 | 47.6 | Lung Ca. (squam.) NCI- | 1.0 | 0.6 | 0.7 |
| | | | | H596 | | | |
| glioma SNB-19 | 1.7 | 1.9 | 0.9 | Mammary gland | 15.3 | 10.4 | 16.6 |
| glioma U251 | 1.6 | 2.4 | 1.5 | Breast ca.* (pl.ef) MCF-7 | 60.7 | 11.2 | 67.8 |
| glioma SF-295 | 35.1 | 62.9 | 30.6 | Breast ca.* (pl.ef) MDA- | 9.2 | 5.5 | 13.0 |
| | | | | MB-231 | | | |
| Heart (Fetal) | 74.2 | 63.3 | 72.7 | Breast ca.* (pl.ef) T47D | 9.3 | 100.0 | 76.8 |
| Heart | 2.4 | 3.7 | 2.1 | Breast Ca. BT-549 | 10.7 | 7.3 | 11.0 |
| Skeletal muscle (Fetal) | 6.3 | 12.9 | 7.0 | Breast Ca. MDA-N | 0.0 | 0.0 | 0.0 |
| Skeletal muscle | 0.0 | 0.0 | 0.0 | Ovary | 11.7 | 18.8 | 10.1 |
| Bone marrow | 0.7 | 0.7 | 0.6 | Ovarian ca. OVCAR-3 | 47.3 | 42.0 | 49.3 |
| Thymus | 0.9 | 1.6 | 1.6 | Ovarian ca. OVCAR-4 | 19.1 | 41.5 | 23.3 |
| Spleen | 0.4 | 0.8 | 0.5 | Ovarian ca. OVCAR-5 | 49.7 | 49.3 | 49.3 |
| Lymph node | 7.0 | 11.1 | 8.1 | Ovarian ca. OVCAR-8 | 15.7 | 25.9 | 19.6 |
| Colorectal | 4.2 | 7.6 | 6.9 | Ovarian ca. IGROV-1 | 12.8 | 21.8 | 21.6 |
| Stomach | 10.8 | 16.7 | 10.7 | Ovarian ca. (ascites) SK- | 66.0 | 64.6 | 100.0 |
| | | | | OV-3 | | | |
| Small intestine | 1.0 | 2.7 | 0.9 | Uterus | 2.6 | 3.5 | 1.4 |
| Colon ca. SW480 | 16.7 | 18.8 | 16.0 | Placenta | 2.9 | 3.3 | 2.7 |
| Colon ca.* SW620 (SW480 | 6.0 | 5.5 | 3.9 | Prostate | 2.5 | 11.2 | 4.5 |
| met) | | | | | | | |
| Colon ca. HT29 | 0.5 | 0.2 | 0.6 | Prostate ca.* (bone met) | 20.4 | 24.0 | 14.7 |
| | | | | PC-3 | | | |
| Colon ca. HCT-116 | 9.2 | 12.2 | 5.4 | Testis | 3.6 | 3.8 | 3.7 |
| Colon ca. CaCo-2 | 23.7 | 14.2 | 16.4 | Melanoma Hs688(A).T | 0.8 | 0.2 | 0.4 |
| CC Well to Mod Duff | 20.6 | 15.8 | 16.4 | Melanoma* (met) | 0.1 | 0.0 | 0.6 |
| (ODO3866) | | | | Hs688(B).T | | | |
| Colon ca. HCC-2998 | 35.1 | 26.6 | 31.4 | Melanoma UACC-62 | 4.4 | 5.8 | 4.2 |
| Gastric ca. (liver met) NCI- | 100.0 | 62.9 | 92.7 | Melanoma M14 | 16.8 | 20.0 | 11.9 |
| N87 | | | | | | | |
| Bladder | 1.8 | 3.3 | 1.7 | Melanoma LOX IMVI | 0.1 | 0.0 | 0.0 |
| Trachea | 27.0 | 48.0 | 26.2 | Melanoma* (met) SK- | 20.3 | 35.1 | 17.4 |
| | | | | MEL-5 | | | |
| Kidney | 8.2 | 12.7 | 5.2 | Adipose | 2.5 | 3.0 | 1.6 |

TABLE RE
Panel 2D
Column A - Rel. Exp.(%) Ag1989, Run 149988692
Column B - Rel. Exp.(%) Ag1990, Run 152494531

| Tissue Name | A | B | Tissue Name | A | B |
|---|---|---|---|---|---|
| Normal Colon | 2.7 | 3.7 | Kidney Margin 8120608 | 9.4 | 8.5 |
| CC Well to Mod Diff (ODO3866) | 4.4 | 2.8 | Kidney Cancer 8120613 | 0.3 | 0.3 |
| CC Margin (ODO3866) | 0.1 | 0.1 | Kidney Margin 8120614 | 13.1 | 15.9 |
| CC Gr.2 rectosigmoid (ODO3868) | 0.91 | 0.8 | Kidney Cancer 9010320 | 7.9 | 7.5 |
| CC Margin (ODO3868) | 0.3 | 0.2 | Kidney Margin 9010321 | 12.3 | 12.8 |
| CC Mod Diff (ODO3920) | 4.0 | 3.8 | Normal Uterus | 0.3 | 0.2 |
| CC Margin (ODO3920) | 0.7 | 0.8 | Uterine Cancer 064011 | 8.7 | 9.8 |
| CC Gr.2 ascend colon (ODO3921) | 2.5 | 3.7 | Normal Thyroid | 2.8 | 2.8 |
| CC Margin (ODO3921) | 0.4 | 0.3 | Thyroid Cancer | 10.7 | 10.5 |
| CC from Partial Hepatectomy | 2.8 | 3.4 | Thyroid Cancer A302152 | 1.4 | 1.1 |
| (ODO4309) Mets | | | | | |
| Liver Margin (ODO4309) | 0.1 | 0.0 | Thyroid Margin A302153 | 4.0 | 4.2 |
| Colon mets to lung (ODO4451-01) | 4.4 | 5.3 | Normal Breast | 8.5 | 8.7 |
| Lung Margin (ODO4451-02) | 4.8 | 5.8 | Breast Cancer | 9.9 | 13.1 |
| Normal Prostate 6546-1 | 3.1 | 3.6 | Breast Cancer (ODO4590-01) | 25.7 | 27.7 |
| Prostate Cancer (ODO4410) | 5.9 | 5.4 | Breast Cancer Mets | 23.5 | 28.9 |
| | | | (ODO4590-03) | | |
| Prostate Margin (ODO4410) | 4.0 | 4.9 | Breast Cancer Metastasis | 100.0 | 100.0 |
| Prostate Cancer (ODO4720-01) | 6.0 | 7.2 | Breast Cancer | 20.9 | 20.7 |
| Prostate Margin (ODO4720-02) | 8.1 | 7.6 | Breast Cancer | 9.3 | 7.9 |
| Normal Lung | 9.0 | 10.2 | Breast Cancer 9100266 | 27.5 | 28.1 |
| Lung Met to Muscle (ODO4286) | 4.4 | 5.5 | Breast Margin 9100265 | 7.7 | 7.9 |
| Muscle Margin (ODO4286) | 0.1 | 0.1 | Breast Cancer A209073 | 14.4 | 12.3 |
| Lung Malignant Cancer | 20.2 | 20.9 | Breast Margin A209073 | 8.1 | 7.0 |
| (ODO3126) | | | | | |
| Lung Margin (ODO3126) | 9.2 | 10.4 | Normal Liver | 0.0 | 0.0 |
| Lung Cancer (ODO4404) | 33.0 | 28.7 | Liver Cancer | 0.1 | 0.1 |
| Lung Margin (ODO4404) | 7.3 | 9.8 | Liver Cancer 1025 | 0.1 | 0.2 |
| Lung Cancer (ODO4565) | 17.8 | 18.2 | Liver Cancer 1026 | 2.7 | 2.5 |
| Lung Margin (ODO4565) | 3.0 | 4.1 | Liver Cancer 6004-T | 0.1 | 0.0 |
| Lung Cancer (ODO4237-01) | 4.2 | 4.5 | Liver Tissue 6004-N | 0.2 | 0.3 |
| Lung Margin (ODO4237-02) | 5.3 | 5.6 | Liver Cancer 6005-T | 2.3 | 2.2 |
| Ocular Mel Met to Liver | 1.2 | 1.4 | Liver Tissue 6005-N | 0.1 | 0.1 |
| (ODO4310) | | | | | |
| Liver Margin (ODO4310) | 0.0 | 0.0 | Normal Bladder | 2.0 | 2.5 |
| Melanoma Metastasis | 3.1 | 3.7 | Bladder Cancer | 2.8 | 2.6 |
| Lung Margin (ODO4321) | 9.6 | 9.0 | Bladder Cancer | 5.4 | 7.4 |
| Normal Kidney | 21.6 | 18.4 | Bladder Cancer (ODO4718- | 1.2 | 0.6 |
| | | | 01) | | |
| Kidney Ca, Nuclear grade 2 | 10.2 | 9.6 | Bladder Normal Adjacent | 1.0 | 0.9 |
| (ODO4338) | | | (ODO4718-03) | | |
| Kidney Margin (ODO4338) | 10.2 | 9.0 | Normal Ovary | 1.2 | 1.2 |
| Kidney Ca Nuclear grade 1/2 | 11.2 | 10.4 | Ovarian Cancer | 7.3 | 8.6 |
| (ODO4339) | | | | | |
| Kidney Margin (ODO4339) | 18.2 | 19.2 | Ovarian Cancer (ODO4768- | 16.6 | 14.1 |
| | | | 07) | | |
| Kidney Ca, Clear cell type | 13.2 | 16.0 | Ovary Margin (ODO4768-08) | 0.9 | 0.6 |
| (ODO4340) | | | | | |
| Kidney Margin (ODO4340) | 16.4 | 15.7 | Normal Stomach | 2.2 | 2.1 |
| Kidney Ca, Nuclear grade 3 | 2.3 | 1.5 | Gastric Cancer 9060358 | 0.7 | 0.6 |
| (ODO4348) | | | | | |
| Kidney Margin (ODO4348) | 9.2 | 9.8 | Stomach Margin 9060359 | 1.8 | 2.0 |
| Kidney Cancer (ODO4622-01) | 7.7 | 9.9 | Gastric Cancer 9060395 | 3.8 | 3.5 |
| Kidney Margin (ODO4622-03) | 2.7 | 3.0 | Stomach Margin 9060394 | 2.2 | 2.3 |
| Kidney Cancer (ODO4450-01) | 10.4 | 9.2 | Gastric Cancer 9060397 | 10.9 | 9.0 |
| Kidney Margin (ODO4450-03) | 5.9 | 4.5 | Stomach Margin 9060396 | 0.5 | 0.6 |
| Kidney Cancer 8120607 | 1.2 | 0.8 | Gastric Cancer 064005 | 2.0 | 1.6 |

TABLE RF
Panel 4.1D
Column A - Rel. Exp.(%) Ag1989, Run 248122213

| Tissue Name | A | Tissue Name | A |
|---|---|---|---|
| Secondary Th1 act | 0.5 | HUVEC IL-1beta | 2.4 |
| Secondary Th2 act | 1.3 | HUVEC IFN gamma | 11.0 |
| Secondary Tr1 act | 0.4 | HUVEC TNF alpha + IFN gamma | 0.0 |
| Secondary Th1 rest | 0.0 | HUVEC TNF alpha + IL4 | 0.4 |
| Secondary Th2 rest | 0.0 | HUVEC IL-11 | 2.7 |
| Secondary Tr1 rest | 0.0 | Lung Microvascular EC none | 20.4 |
| Primary Th1 act | 0.0 | Lung Microvascular EC TNF alpha + IL- | 4.8 |
| | | 1beta | |
| Primary Th2 act | 0.4 | Microvascular Dermal EC none | 0.9 |
| Primary Tr1 act | 0.7 | Microsvasular Dermal EC TNF alpha + IL- | 2.2 |
| | | 1beta | |
| Primary Th1 rest | 0.0 | Bronchial epithelium TNF alpha + IL1beta | 50.7 |
| Primary Th2 rest | 0.0 | Small airway epithelium none | 43.2 |
| Primary Tr1 rest | 0.0 | Small airway epithelium TNF alpha + IL- | 85.9 |
| | | 1beta | |
| CD45RA CD4 lymphocyte act | 1.0 | Coronery artery SMC rest | 0.4 |
| CD45RO CD4 lymphocyte act | 0.2 | Coronery artery SMC TNF alpha + IL-1beta | 0.0 |
| CD8 lymphocyte act | 0.0 | Astrocytes rest | 6.0 |
| Secondary CD8 lymphocyte rest | 0.2 | Astrocytes TNF alpha + IL-1beta | 5.5 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.2 |
| 2ry Th1/Th2/Tr1 anti-CD95 | 0.0 | CCD1106 (Keratinocytes) none | 92.0 |
| CH11 | | | |
| LAK cells rest | 1.7 | CCD1106 (Keratinocytes) TNF alpha + IL- | 57.0 |
| | | 1beta | |
| LAK cells IL-2 | 0.0 | Liver cirrhosis | 1.5 |
| LAK cells IL-2 + IL-12 | 0.0 | NCI-H292 none | 24.7 |
| LAK cells IL-2 + IFN gamma | 0.0 | NCI-H292 IL-4 | 25.2 |
| LAK cells IL-2 + IL-18 | 0.2 | NCI-H292 IL-9 | 25.7 |
| LAK cells PMA/ionomycin | 1.6 | NCI-H292 IL-13 | 40.9 |
| NK Cells IL-2 rest | 1.4 | NCI-H292 IFN gamma | 14.1 |
| Two Way MLR 3 day | 0.1 | HPAEC none | 0.8 |
| Two Way MLR 5 day | 0.0 | HPAEC TNF alpha + IL-1beta | 1.0 |
| Two Way MLR 7 day | 0.0 | Lung fibroblast none | 0.6 |
| PBMC rest | 0.1 | Lung fibroblast TNF alpha + IL-1beta | 0.0 |
| PBMC PWM | 0.0 | Lung fibroblast IL-4 | 0.1 |
| PBMC PHA-L | 0.2 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.3 |
| B lymphocytes PWM | 0.0 | Dermal fibroblast CCD1070 rest | 3.2 |
| B lymphocytes CD40L and IL-4 | 0.1 | Dermal fibroblast CCD1070 TNF alpha | 2.1 |
| EOL-1 dbcAMP | 0.0 | Dermal fibroblast CCD1070 IL-1beta | 0.6 |
| EOL-1 dbcAMP | 0.0 | Dermal fibroblast IFN gamma | 0.4 |
| PMA/ionomycin | | | |
| Dendritic cells none | 14.1 | Dermal fibroblast IL-4 | 0.3 |
| Dendritic cells LPS | 1.6 | Dermal Fibroblasts rest | 1.7 |
| Dendritic cells anti-CD40 | 3.0 | Neutrophils TNFa + LPS | 0.0 |
| Monocytes rest | 0.0 | Neutrophils rest | 0.0 |
| Monocytes LPS | 2.9 | Colon | 0.5 |
| Macrophages rest | 6.0 | Lung | 2.9 |
| Macrophages LPS | 0.9 | Thymus | 1.0 |
| HUVEC none | 3.0 | Kidney | 100.0 |
| HUVEC starved | 3.1 | | |

AI_comprehensive panel_v1.0 Summary: Ag1989/Ag1990 The highest expression of this gene was detected in an osteoarthritic bone sample (CT=29). The expression of this gene was upregulated in bone, synovium, synovial fluid, and cartilage in patients with rheumatoid arthritis (RA) (CTs=29-30) compared to normal controls (CTs=32-38). In addition, expression of this gene was upregulated in lung samples from patients with asthma compared to normal lung controls (CT values=29-30 for patients with asthma versus 34-36 for normal controls). Therefore, expression of this gene is useful as a marker to identify samples from patients with rheumatoid arthritis or asthma. Therapeutic modulation of this gene, expressed protein and/or use of antibodies or small molecule drugs targeting the gene or gene product are useful in the treatment of rheumatoid arthritis or asthma.

Panel 1.3D Summary: Ag1989/Ag1990 This gene was strongly expressed in most tumor cell lines in this panel, while the expression in most normal tissues was moderate or low. Expression of this gene was up-regulated in a subset of glioma, astrocytoma, pancreatic, colon, kidney, lung, breast and ovarian cancer cell lines. Therefore, expression of this gene is useful in the detection and diagnosis of these types of cancer. Therefore, therapeutic modulation of this gene, expressed protein and/or use of antibodies or small molecule drugs targeting the gene or gene product is useful in the treatment of brain, pancreatic, colon, kidney, lung, breast and ovarian cancer.

Panel 2D Summary: Ag1989/Ag1990 The highest expression of this gene was detected in a metastatic breast cancer sample (CT=25). For all tumor sites there were several cases where the tumor tissues strongly overexpressed the CG53971-01 gene as compared to the normal adjacent controls, especially for lung, breast and ovarian cancers, indicating a role in tumorgenesis. Therefore, therapeutic modulation of this gene, expressed protein and/or use of antibodies or small molecule drugs targeting the gene or gene product is useful in the treatment of cancers including brain, pancreatic, colon, kidney, lung, breast and ovarian cancers.

Panel 4.1D Summary: Ag1989 Expression of this gene was highest in kidney (CT=28.2). In addition, this gene was expressed at moderate levels in the lung cell line NCI-H292 and keratinocytes, irrespective of treatment. Expression of this gene was also up-regulated approximately two-fold in activated small airway epithelium, consistent with a potential role for the CG53971-01 gene in asthma and emphysema. Therapeutic modulation of this gene, expressed protein and/or use of antibodies or small molecule drugs targeting the gene or gene product is useful in the treatment of asthma or emphysema.

S. CG51983-05: A Disintegrin and Metalloproteinase

Expression of gene CG51983-05 was assessed using the primer-probe set Ag1322, described in Table SA. Results of the RTQ-PCR runs are shown in Table SB.

TABLE SA
Probe Name Ag1322

| | | | Start | SEQ ID |
|---|---|---|---|---|
| Primers | Sequences | Length | Position | No |
| Forward | 5′-ggtatgtgcctgccctattatt-3′ | 22 | 922 | 1211 |
| Probe | TET-5′-ccaccagtatcattaaggatcttttacc | 30 | 944 | 1212 |
| | tg-3′-TAMRA | | | |
| Reverse | 5′-gccattctgtttgcaattatgt-3′ | 22 | 980 | 1213 |

TABLE SB
Panel 1.2
Column A - Rel. Exp.(%) Ag1322, Run 133804727

| Tissue Name | A | Tissue Name | A |
|---|---|---|---|
| Endothelial cells | 0.0 | Renal ca. 786-0 | 0.0 |
| Heart (Fetal) | 0.0 | Renal ca. A498 | 0.0 |
| Pancreas | 0.2 | Renal ca. RXF 393 | 0.0 |
| Pancreatic ca. CAPAN 2 | 0.0 | Renal ca. ACHN | 0.0 |
| Adrenal gland | 0.2 | Renal ca. UO-31 | 0.0 |
| Thyroid | 0.0 | Renal ca. TK-10 | 0.0 |
| Salivary gland | 0.1 | Liver | 0.0 |
| Pituitary gland | 0.0 | Liver (fetal) | 0.2 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.1 |
| Brain (cerebellum) | 0.0 | Lung ca. (small cell) LX-1 | 0.0 |
| Brain (hippocampus) | 0.0 | Lung ca. (small cell) NCI-H69 | 0.0 |
| Brain (thalamus) | 0.0 | Lung ca. (s. cell var.) SHP-77 | 0.0 |
| Cerebral Cortex | 0.0 | Lung ca. (large cell) NCI-H460 | 0.0 |
| Spinal cord | 0.1 | Lung ca. (non-sm. cell) A549 | 0.0 |
| glio/astro U87-MG | 0.0 | Lung ca. (non-s. cell) NCI-H23 | 0.0 |
| glio/astro U-118-MG | 0.0 | Lung ca. (non-s. cell) HOP-62 | 0.0 |
| astrocytoma SW1783 | 0.0 | Lung ca. (non-s. cl) NCI-H522 | 0.0 |
| neuro*; met SK-N-AS | 0.0 | Lung ca. (squam.) SW 900 | 0.0 |
| astrocytoma SF-539 | 0.0 | Lung ca. (squam.) NCI-H596 | 0.0 |
| astrocytoma SNB-75 | 0.0 | Mammary gland | 0.0 |
| glioma SNB-19 | 0.0 | Breast ca.* (pl.ef) MCF-7 | 0.0 |
| glioma U251 | 0.1 | Breast ca.* (pl.ef) MDA-MB-231 | 0.0 |
| glioma SF-295 | 0.0 | Breast ca.* (pl.ef) T47D | 0.0 |
| Heart | 0.0 | Breast ca. BT-549 | 0.0 |
| Skeletal muscle | 0.0 | Breast ca. MDA-N | 0.0 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.1 | Ovarian ca. OVCAR-5 | 0.0 |
| Colorectal | 0.0 | Ovarian ca. OVCAR-8 | 0.0 |
| Stomach | 0.1 | Ovarian ca. IGROV-1 | 0.0 |
| Small intestine | 0.0 | Ovarian ca. (ascites) SK-OV-3 | 0.0 |
| Colon ca. SW480 | 0.0 | Uterus | 0.0 |
| Colon ca.* SW620 | 0.0 | Placenta | 0.0 |
| (SW480 met) | | | |
| Colon ca. HT29 | 0.0 | Prostate | 2.1 |
| Colon ca. HCT-116 | 0.0 | Prostate ca.* (bone met) PC-3 | 0.0 |
| Colon ca. CaCo-2 | 0.0 | Testis | 100.0 |
| CC Well to Mod Diff | 0.0 | Melanoma Hs688(A).T | 0.0 |
| (ODO3866) | | | |
| Colon ca. HCC-2998 | 0.0 | Melanoma* (met) Hs688(B).T | 0.0 |
| Gastric ca. (liver met) | 0.0 | Melanoma UACC-62 | 0.0 |
| NCI-N87 | | | |
| Bladder | 0.1 | Melanoma M14 | 0.0 |
| Trachea | 0.0 | Melanoma LOX IMVI | 0.0 |
| Kidney | 0.0 | Melanoma* (met) SK-MEL-5 | 0.0 |
| Kidney (fetal) | 0.0 | | |

Panel 1.2 Summary: Ag1322 Expression of this gene was highest in testis (CT value=29). Low expression was also seen in prostate (CT value=34.6). The gene or encoded protein is useful as a marker for these tissues. This gene encodes a protein with homology to ADAM proteins, which are membrane disintegrin-metalloproteases. The expression of several other ADAM proteins has been shown to be testis-specific and these proteins are thought to play a role in fertilization (Hooft van Huijsduijnen R. (1998) Gene 206: 273-282). Therapeutic modulation of this gene, expressed protein and/or use of antibodies or small molecule drugs targeting the gene or gene product is useful in the treatment of diseases of the prostate and testis, including infertility.

T. CG53390-02: Olfactory Receptor

Expression of gene CG53390-02 was assessed using the primer-probe sets Ag1588 and Ag2015, described in Tables TA and TB. Results of the RTQ-PCR runs are shown in Tables TC, TD and TE.

TABLE TA
Probe Name Ag1588

| | | | Start | SEQ ID |
|---|---|---|---|---|
| Primers | Sequences | Length | Position | No |
| Forward | 5′-aagctctcctgtgcagatacct-3′ | 22 | 582 | 1214 |
| Probe | TET-5′-ctacgagatggcgctgtccacct-3′- | 23 | 608 | 1215 |
| | TAMRA | | | |
| Reverse | 5′-aaagagggagcattaggatcag-3′ | 22 | 639 | 1216 |

TABLE TB
Probe Name Ag2015

| | | | Start | SEQ ID |
|---|---|---|---|---|
| Primers | Sequences | Length | Position | No |
| Forward | 5′-aagctctcctgtgcagatacct-3′ | 22 | 582 | 1217 |
| Probe | TET-5′-ctacgagatggcgctgtccacct-3′- | 23 | 608 | 1218 |
| | TAMRA | | | |
| Reverse | 5′-aaagagggagcattaggatcag-3′ | 22 | 639 | 1219 |

TABLE TC
Panel 1.3D
Column A - Rel. Exp. (%) Ag1588, Run 165529897
Column B - Rel. Exp. (%) Ag2015, Run 147837991
Column C - Rel. Exp. (%) Ag2015, Run 152152893

| Tissue Name | A | B | C | Tissue Name | A | B | C |
|---|---|---|---|---|---|---|---|
| Liver adenocarcinoma | 0.0 | 0.0 | 0.0 | Kidney (fetal) | 0.0 | 0.0 | 0.0 |
| Pancreas | 0.0 | 0.0 | 0.0 | Renal ca. 786-0 | 0.0 | 0.0 | 0.0 |
| Pancreatic ca. CAPAN 2 | 0.0 | 0.0 | 0.0 | Renal ca. A498 | 0.0 | 0.0 | 0.0 |
| Adrenal gland | 0.0 | 0.0 | 0.6 | Renal ca. RXF 393 | 0.0 | 0.8 | 0.0 |
| Thyroid | 0.0 | 0.0 | 0.0 | Renal ca. ACHN | 0.0 | 0.0 | 1.2 |
| Salivary gland | 0.0 | 0.0 | 0.0 | Renal ca. UO-31 | 0.0 | 0.0 | 0.0 |
| Pituitary gland | 0.0 | 0.0 | 0.0 | Renal ca. TK-10 | 0.0 | 0.0 | 1.2 |
| Brain (fetal) | 0.0 | 0.0 | 0.0 | Liver | 0.0 | 0.0 | 0.0 |
| Brain (whole) | 0.0 | 0.0 | 0.0 | Liver (fetal) | 0.0 | 0.0 | 0.0 |
| Brain (amygdala) | 0.0 | 0.0 | 0.0 | Liver ca. (hepatoblast) | 0.0 | 0.0 | 0.0 |
| | | | | HepG2 | | | |
| Brain (cerebellum) | 0.0 | 0.0 | 0.0 | Lung | 0.0 | 0.3 | 0.0 |
| Brain (hippocampus) | 0.0 | 0.0 | 0.0 | Lung (fetal) | 0.0 | 0.0 | 0.0 |
| Brain (substantia nigra) | 0.0 | 0.0 | 0.0 | Lung ca. (small cell) LX-1 | 0.0 | 0.0 | 0.0 |
| Brain (thalamus) | 0.0 | 0.0 | 0.0 | Lung ca. (small cell) NCI- | 0.0 | 0.0 | 0.0 |
| | | | | H69 | | | |
| Cerebral Cortex | 0.0 | 0.0 | 2.6 | Lung ca. (s. cell var.) SHP- | 100.0 | 100.0 | 100.0 |
| | | | | 77 | | | |
| Spinal cord | 0.0 | 0.0 | 0.0 | Lung ca. (large cell) NCI- | 0.0 | 0.0 | 0.0 |
| | | | | H460 | | | |
| glio/astro U87-MG | 0.0 | 0.0 | 0.0 | Lung ca. (non-sm. cell) | 0.0 | 0.0 | 0.0 |
| | | | | A549 | | | |
| glio/astro U-118-MG | 0.0 | 0.0 | 1.1 | Lung ca. (non-s. cell) NCI- | 1.7 | 1.6 | 2.1 |
| | | | | H23 | | | |
| astrocytoma SW1783 | 1.4 | 0.3 | 0.0 | Lung ca. (non-s. cell) HOP- | 0.0 | 0.0 | 0.0 |
| | | | | 62 | | | |
| neuro*; met SK-N-AS | 0.0 | 0.0 | 0.0 | Lung ca. (non-s. cl) NCI- | 0.0 | 0.0 | 0.0 |
| | | | | H522 | | | |
| astrocytoma SF-539 | 0.0 | 0.0 | 0.0 | Lung ca. (squam.) SW 900 | 0.0 | 0.0 | 0.0 |
| astrocytoma SNB-75 | 0.0 | 0.0 | 1.1 | Lung ca. (squam.) NCI- | 0.0 | 0.0 | 0.0 |
| | | | | H596 | | | |
| glioma SNB-19 | 0.0 | 0.3 | 2.0 | Mammary gland | 0.0 | 0.0 | 1.3 |
| glioma U251 | 0.7 | 0.0 | 0.0 | Breast ca.* (pl. ef) MCF-7 | 0.0 | 0.5 | 0.0 |
| glioma SF-295 | 0.0 | 0.0 | 0.0 | Breast ca.* (pl. ef) MDA- | 0.0 | 0.0 | 0.0 |
| | | | | MB-231 | | | |
| Heart (Fetal) | 0.0 | 0.0 | 0.0 | Breast ca.* (pl. ef) T47D | 0.0 | 0.4 | 0.0 |
| Heart | 0.0 | 0.0 | 0.7 | Breast ca. BT-549 | 0.0 | 0.0 | 0.6 |
| Skeletal muscle (Fetal) | 0.0 | 0.0 | 0.6 | Breast ca. MDA-N | 0.0 | 0.9 | 0.0 |
| Skeletal muscle | 0.0 | 0.0 | 0.0 | Ovary | 0.0 | 0.0 | 0.0 |
| Bone marrow | 0.0 | 0.0 | 0.0 | Ovarian ca. OVCAR-3 | 0.0 | 0.8 | 0.0 |
| Thymus | 0.0 | 0.3 | 0.0 | Ovarian ca. OVCAR-4 | 0.0 | 0.0 | 0.0 |
| Spleen | 0.8 | 0.0 | 0.0 | Ovarian ca. OVCAR-5 | 0.0 | 0.4 | 0.0 |
| Lymph node | 0.0 | 0.0 | 0.0 | Ovarian ca. OVCAR-8 | 0.7 | 0.0 | 0.0 |
| Colorectal | 0.8 | 0.7 | 2.1 | Ovarian ca. IGROV-1 | 0.0 | 0.0 | 0.0 |
| Stomach | 0.0 | 0.0 | 0.0 | Ovarian ca. (ascites) SK- | 0.0 | 0.0 | 0.0 |
| | | | | OV-3 | | | |
| Small intestine | 0.0 | 0.0 | 0.6 | Uterus | 0.0 | 0.4 | 0.0 |
| Colon ca. SW480 | 0.0 | 0.0 | 0.0 | Placenta | 0.0 | 0.8 | 2.4 |
| Colon ca.* SW620 (SW480 | 0.0 | 0.0 | 1.9 | Prostate | 0.0 | 0.0 | 0.0 |
| met) | | | | | | | |
| Colon ca. HT29 | 0.0 | 0.0 | 0.0 | Prostate ca.* (bone met) | 0.0 | 0.0 | 0.0 |
| | | | | PC-3 | | | |
| Colon ca. HCT-116 | 0.0 | 0.7 | 0.0 | Testis | 0.0 | 1.6 | 0.5 |
| Colon ca. CaCo-2 | 0.0 | 0.0 | 38.7 | Melanoma Hs688(A).T | 0.0 | 0.0 | 0.0 |
| CC Well to Mod Diff | 0.0 | 0.0 | 1.6 | Melanoma* (met) | 0.0 | 0.0 | 0.0 |
| (ODO3866) | | | | Hs688(B).T | | | |
| Colon ca. HCC-2998 | 0.0 | 0.0 | 0.6 | Melanoma UACC-62 | 0.0 | 0.0 | 0.0 |
| Gastric ca. (liver met) NCI- | 0.0 | 0.0 | 0.7 | Melanoma M14 | 0.0 | 0.0 | 0.0 |
| N87 | | | | | | | |
| Bladder | 0.0 | 0.0 | 0.0 | Melanoma LOX IMVI | 0.0 | 0.0 | 1.1 |
| Trachea | 0.0 | 0.0 | 0.0 | Melanoma* (met) SK- | 0.0 | 0.0 | 1.4 |
| | | | | MEL-5 | | | |
| Kidney | 0.0 | 0.0 | 0.0 | Adipose | 0.0 | 0.0 | 0.0 |

TABLE TD
Panel 2D
Column A - Rel. Exp.(%) Ag2015, Run 152152937

| Tissue Name | A | Tissue Name | A |
|---|---|---|---|
| Normal Colon | 10.6 | Kidney Margin 8120608 | 0.0 |
| CC Well to Mod Diff (ODO3866) | 32.5 | Kidney Cancer 8120613 | 0.0 |
| CC Margin (ODO3866) | 1.1 | Kidney Margin 8120614 | 25.9 |
| CC Gr.2 rectosigmoid (ODO3868) | 20.6 | Kidney Cancer 9010320 | 0.0 |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon (ODO3921) | 0.0 | Normal Thyroid | 0.0 |
| CC Margin (ODO3921) | 48.6 | Thyroid Cancer | 0.0 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 0.0 | Normal Breast | 0.0 |
| Lung Margin (OD04451-02) | 0.0 | Breast Cancer | 0.0 |
| Normal Prostate 6546-1 | 0.0 | Breast Cancer (OD04590-01) | 0.0 |
| Prostate Cancer (OD04410) | 0.0 | Breast Cancer Mets (OD04590-03) | 0.0 |
| Prostate Margin (OD04410) | 0.0 | Breast Cancer Metastasis | 0.0 |
| Prostate Cancer (OD04720-01) | 0.0 | Breast Cancer | 0.0 |
| Prostate Margin (OD04720-02) | 0.0 | Breast Cancer | 0.0 |
| Normal Lung | 25.0 | Breast Cancer 9100266 | 0.0 |
| Lung Met to Muscle (ODO4286) | 0.0 | Breast Margin 9100265 | 0.0 |
| Muscle Margin (ODO4286) | 0.0 | Breast Cancer A209073 | 21.3 |
| Lung Malignant Cancer (OD03126) | 0.0 | Breast Margin A209073 | 9.7 |
| Lung Margin (OD03126) | 0.0 | Normal Liver | 0.0 |
| Lung Cancer (OD04404) | 0.0 | Liver Cancer | 0.8 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 11.9 |
| Lung Cancer (OD04237-01) | 0.0 | Liver Tissue 6004-N | 100.0 |
| Lung Margin (OD04237-02) | 0.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 23.7 |
| Melanoma Metastasis | 0.0 | Bladder Cancer | 19.9 |
| Lung Margin (OD04321) | 0.0 | Bladder Cancer | 60.7 |
| Normal Kidney | 0.0 | Bladder Cancer (OD04718-01) | 0.0 |
| Kidney Ca, Nuclear grade 2 (OD04338) | 0.0 | Bladder Normal Adjacent | 0.0 |
| | | (OD04718-03) | |
| Kidney Margin (OD04338) | 0.0 | Normal Ovary | 0.0 |
| Kidney Ca Nuclear grade ½ (OD04339) | 0.0 | Ovarian Cancer | 0.0 |
| Kidney Margin (OD04339) | 0.0 | Ovarian Cancer (OD04768-07) | 26.1 |
| Kidney Ca, Clear cell type (OD04340) | 0.0 | Ovary Margin (OD04768-08) | 0.0 |
| Kidney Margin (OD04340) | 0.0 | Normal Stomach | 0.0 |
| Kidney Ca, Nuclear grade 3 (OD04348) | 0.0 | Gastric Cancer 9060358 | 21.0 |
| Kidney Margin (OD04348) | 0.0 | Stomach Margin 9060359 | 0.0 |
| Kidney Cancer (OD04622-01) | 0.0 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 0.0 |
| Kidney Margin (OD04450-03) | 24.7 | Stomach Margin 9060396 | 0.0 |
| Kidney Cancer 8120607 | 0.0 | Gastric Cancer 064005 | 19.8 |

TABLE TE
Panel 4D
Column A - Rel. Exp. (%) Ag1588, Run 165373604
Column B - Rel. Exp. (%) Ag2015, Run 152153145
Column C - Rel. Exp. (%) Ag2015, Run 152685551

| Tissue Name | A | B | C | Tissue Name | A | B | C |
|---|---|---|---|---|---|---|---|
| Secondary Th1 act | 0.0 | 0.0 | 40.1 | HUVEC IL-1beta | 0.0 | 0.0 | 0.0 |
| Secondary Th2 act | 26.4 | 25.3 | 26.1 | HUVEC IFN gamma | 0.0 | 0.0 | 0.0 |
| Secondary Tr1 act | 12.3 | 58.6 | 0.0 | HUVEC TNFalpha + IFN | 0.0 | 0.0 | 0.0 |
| | | | | gamma | | | |
| Secondary Th1 rest | 0.0 | 11.5 | 0.0 | HUVEC TNFalpha + IL4 | 0.0 | 0.0 | 0.0 |
| Secondary Th2 rest | 16.6 | 43.8 | 31.0 | HUVEC IL-11 | 0.0 | 0.0 | 0.0 |
| Secondary Tr1 rest | 42.6 | 24.5 | 14.2 | Lung Microvascular EC none | 0.0 | 0.0 | 0.0 |
| Primary Th1 act | 0.0 | 17.3 | 25.0 | Lung Microvascular EC | 0.0 | 0.0 | 0.0 |
| | | | | TNFalpha + IL-1beta | | | |
| Primary Th2 act | 0.0 | 0.0 | 0.0 | Microvascular Dermal EC | 0.0 | 0.0 | 0.0 |
| | | | | none | | | |
| Primary Tr1 act | 0.0 | 27.5 | 0.0 | Microsvasular Dermal EC | 0.0 | 0.0 | 24.0 |
| | | | | TNFalpha + IL-1beta | | | |
| Primary Th1 rest | 18.6 | 69.7 | 12.3 | Bronchial epithelium | 0.0 | 0.0 | 0.0 |
| | | | | TNFalpha + IL1beta | | | |
| Primary Th2 rest | 13.9 | 31.6 | 0.0 | Small airway epithelium none | 0.0 | 0.0 | 0.0 |
| Primary Tr1 rest | 0.0 | 0.0 | 0.0 | Small airway epithelium | 0.0 | 0.0 | 0.0 |
| | | | | TNFalpha + IL-1beta | | | |
| CD45RA CD4 | 0.0 | 0.0 | 12.6 | Coronery artery SMC rest | 0.0 | 44.4 | 0.0 |
| lymphocyte act | | | | | | | |
| CD45RO CD4 | 23.0 | 0.0 | 0.0 | Coronery artery SMC | 0.0 | 0.0 | 0.0 |
| lymphocyte act | | | | TNFalpha + IL-1beta | | | |
| CD8 lymphocyte act | 0.0 | 0.0 | 0.0 | Astrocytes rest | 0.0 | 0.0 | 27.5 |
| Secondary CD8 | 0.0 | 0.0 | 0.0 | Astrocytes TNFalpha + IL- | 0.0 | 0.0 | 0.0 |
| lymphocyte rest | | | | 1beta | | | |
| Secondary CD8 | 27.5 | 0.0 | 0.0 | KU-812 (Basophil) rest | 0.0 | 0.0 | 0.0 |
| lymphocyte act | | | | | | | |
| CD4 lymphocyte none | 13.1 | 0.0 | 13.2 | KU-812 (Basophil) | 0.0 | 0.0 | 41.8 |
| | | | | PMA/ionomycin | | | |
| 2ry Th1/Th2/Tr1 anti- | 0.0 | 23.2 | 29.5 | CCD1106 (Keratinocytes) | 0.0 | 0.0 | 0.0 |
| CD95 CH11 | | | | none | | | |
| LAK cells rest | 15.2 | 12.0 | 0.0 | CCD1106 (Keratinocytes) | 0.0 | 0.0 | 24.7 |
| | | | | TNFalpha + IL-1beta | | | |
| LAK cells IL-2 | 0.0 | 31.6 | 0.0 | Liver cirrhosis | 100.0 | 100.0 | 100.0 |
| LAK cells IL-2 + IL-12 | 16.6 | 33.2 | 0.0 | Lupus kidney | 0.0 | 0.0 | 0.0 |
| LAK cells IL-2 + IFN | 0.0 | 0.0 | 15.6 | NCI-H292 none | 0.0 | 0.0 | 0.0 |
| gamma | | | | | | | |
| LAK cells IL-2 + IL-18 | 0.0 | 0.0 | 25.3 | NCI-H292 IL-4 | 0.0 | 26.6 | 0.0 |
| LAK cells | 0.0 | 0.0 | 29.7 | NCI-H292 IL-9 | 0.0 | 0.0 | 0.0 |
| PMA/ionomycin | | | | | | | |
| NK Cells IL-2 rest | 35.6 | 25.5 | 20.3 | NCI-H292 IL-13 | 0.0 | 0.0 | 0.0 |
| Two Way MLR 3 day | 0.0 | 0.0 | 0.0 | NCI-H292 IFN gamma | 0.0 | 23.3 | 0.0 |
| Two Way MLR 5 day | 0.0 | 0.0 | 24.1 | HPAEC none | 0.0 | 0.0 | 0.0 |
| Two Way MLR 7 day | 0.0 | 0.0 | 0.0 | HPAEC TNFalpha + IL-1 | 0.0 | 0.0 | 0.0 |
| | | | | beta | | | |
| PBMC rest | 0.0 | 12.3 | 0.0 | Lung fibroblast none | 0.0 | 14.2 | 0.0 |
| PBMC PWM | 10.1 | 0.0 | 0.0 | Lung fibroblast TNFalpha + | 0.0 | 0.0 | 0.0 |
| | | | | IL-1 beta | | | |
| PBMC PHA-L | 0.0 | 0.0 | 24.3 | Lung fibroblast IL-4 | 0.0 | 25.7 | 0.0 |
| Ramos (B cell) none | 0.0 | 0.0 | 0.0 | Lung fibroblast IL-9 | 0.0 | 0.0 | 0.0 |
| Ramos (B cell) | 0.0 | 16.3 | 0.0 | Lung fibroblast IL-13 | 0.0 | 19.3 | 0.0 |
| ionomycin | | | | | | | |
| B lymphocytes PWM | 0.0 | 13.7 | 43.2 | Lung fibroblast IFN gamma | 0.0 | 0.0 | 0.0 |
| B lymphocytes CD40L | 0.0 | 20.2 | 0.0 | Dermal fibroblast CCD1070 | 0.0 | 0.0 | 30.1 |
| and IL-4 | | | | rest | | | |
| EOL-1 dbcAMP | 0.0 | 0.0 | 0.0 | Dermal fibroblast CCD1070 | 57.4 | 40.9 | 25.7 |
| | | | | TNFalpha | | | |
| EOL-1 dbcAMP | 0.0 | 11.0 | 0.0 | Dermal fibroblast CCD1070 | 0.0 | 0.0 | 0.0 |
| PMA/ionomycin | | | | IL-1 beta | | | |
| Dendritic cells none | 0.0 | 0.0 | 0.0 | Dermal fibroblast | 0.0 | 0.0 | 0.0 |
| | | | | gamma | | | |
| Dendritic cells LPS | 0.0 | 0.0 | 0.0 | Dermal fibroblast IL-4 | 0.0 | 0.0 | 0.0 |
| Dendritic cells anti- | 0.0 | 22.5 | 0.0 | IBD Colitis 2 | 0.0 | 0.0 | 0.0 |
| CD40 | | | | | | | |
| Monocytes rest | 0.0 | 0.0 | 23.2 | IBD Crohn's | 0.0 | 0.0 | 24.8 |
| Monocytes LPS | 0.0 | 0.0 | 22.7 | Colon | 0.0 | 0.0 | 29.9 |
| Macrophages rest | 0.0 | 0.0 | 24.5 | Lung | 0.0 | 22.5 | 56.3 |
| Macrophages LPS | 0.0 | 0.0 | 0.0 | Thymus | 14.7 | 0.0 | 0.0 |
| HUVEC none | 0.0 | 18.7 | 0.0 | Kidney | 0.0 | 0.0 | 0.0 |
| HUVEC starved | 0.0 | 0.0 | 0.0 | | | | |

Panel 1.3D Summary: Ag1588/Ag2015 Highest expression was detected in a lung cancer cell line (CTs=29).

Panel 2D Summary: Ag2015 Highest expression was detected in normal liver tissue (CT=33)

Panel 4D Summary: Ag2015 Highest expression was detected in liver cirrhosis (CTs=32-34).

U. CG53530-03: Olfactory Receptor

Expression of full-length physical clone CG53530-03 was assessed using the primer-probe set g1194 described in Table UA. Results of the RTQ-PCR runs are shown in Table UB

TABLE UA
Probe Name Ag1194

| | | | Start | SEQ ID |
|---|---|---|---|---|
| Primers | Sequences | Length | Position | No |
| Forward | 5′-gggaaaccttcttattgtggtca-3′ | 23 | 126 | 1220 |
| Probe | TET-5′-tgacctccgacccacacctgca-3′- | 22 | 152 | 1221 |
| | TAMRA | | | |
| Reverse | 5′-gattgcccaagagaaaatacatgg-3′ | 24 | 179 | 1222 |

TABLE UB
Panel 1.3D
Column A - Rel. Exp.(%) Ag1194, Run 153594145

| Tissue Name | A | Tissue Name | A |
|---|---|---|---|
| Liver adenocarcinoma | 0.0 | Kidney (fetal) | 0.0 |
| Pancreas | 56.3 | Renal ca. 786-0 | 0.0 |
| Pancreatic ca. | 0.0 | Renal ca. A498 | 0.0 |
| CAPAN 2 | | | |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 5.2 | Renal ca. TK-10 | 0.0 |
| Brain (fetal) | 7.0 | Liver | 0.0 |
| Brain (whole) | 0.0 | Liver (fetal) | 0.0 |
| Brain (amygdala) | 10.7 | Liver ca. (hepatoblast) HepG2 | 10.5 |
| Brain (cerebellum) | 0.0 | Lung | 0.0 |
| Brain (hippocampus) | 0.0 | Lung (fetal) | 0.0 |
| Brain (substantia | 0.0 | Lung ca. (small cell) LX-1 | 0.0 |
| nigra) | | | |
| Brain (thalamus) | 0.0 | Lung ca. (small cell) NCI-H69 | 0.0 |
| Cerebral Cortex | 9.5 | Lung ca. (s. cell var.) SHP-77 | 5.5 |
| Spinal cord | 0.0 | Lung ca. (large cell) NCI-H460 | 0.0 |
| glio/astro U87-MG | 0.0 | Lung ca. (non-sm. cell) A549 | 0.0 |
| glio/astro U-118-MG | 0.0 | Lung ca. (non-s. cell) NCI-H23 | 0.0 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA-MB-231 | 0.0 |
| Heart (Fetal) | 0.0 | Breast ca.* (pl.ef) T47D | 0.0 |
| Heart | 0.0 | Breast ca. BT-549 | 0.0 |
| Skeletal muscle (Fetal) | 18.3 | Breast ca. MDA-N | 0.0 |
| Skeletal muscle | 0.0 | Ovary | 7.5 |
| Bone marrow | 0.0 | Ovarian ca. OVCAR-3 | 0.0 |
| Thymus | 0.0 | Ovarian ca. OVCAR-4 | 0.0 |
| Spleen | 14.8 | Ovarian ca. OVCAR-5 | 0.0 |
| Lymph node | 0.0 | Ovarian ca. OVCAR-8 | 0.0 |
| Colorectal | 100.0 | Ovarian ca. IGROV-1 | 0.0 |
| Stomach | 0.0 | Ovarian ca. (ascites) SK-OV-3 | 0.0 |
| Small intestine | 0.0 | Uterus | 0.0 |
| Colon ca. SW480 | 0.0 | Placenta | 4.4 |
| Colon ca.* SW620 | 0.0 | Prostate | 0.0 |
| (SW480 met) | | | |
| Colon ca. HT29 | 0.0 | Prostate ca.* (bone met) PC-3 | 0.0 |
| Colon ca. HCT-116 | 0.0 | Testis | 28.5 |
| Colon ca. CaCo-2 | 0.0 | Melanoma Hs688(A).T | 0.0 |
| CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 57.4 | Melanoma LOX IMVI | 4.7 |
| Trachea | 0.0 | Melanoma* (met) SK-MEL-5 | 0.0 |
| Kidney | 0.0 | Adipose | 0.0 |

Panel 1.3D Summary: Ag1194 Expression of the CG53530-03 gene was highest in colon (CT=32.4) and was primarily associated with normal tissue. Significant gene expression was also detected in bladder, pancreas, and testis. Expression of this gene was downregulated in colon and pancreatic cancer cell lines when compared to the appropriate normal controls. Therapeutic modulation of the activity of this gene or its protein product using nucleic acid, protein, antibody or small molecule drugs is useful in the treatment of colon or pancreatic cancer.

Panel 2D Summary: Ag1194 Prominent expression was detected in normal colon and bladder tissues, in agreement with the results in Panel 1.3D. This gene was expressed at higher levels in normal colon and bladder tissues than in malignant colon and bladder tissues. Targeting this gene or its protein product with small molecule, antibody, or protein therapeutics is useful in the treatment of colon and bladder cancers.

V. CG53719-02: Olfactory Receptor

Expression of gene CG53719-02 was assessed using the primer-probe set Ag379, described in Table VA. Results of the RTQ-PCR runs are shown in Tables VB and VC.

TABLE VA
Probe Name Ag379

| | | | Start | SEQ ID |
|---|---|---|---|---|
| Primers | Sequences | Length | Position | No |
| Forward | 5′-tgtgtccgattagtggccttc-3′ | 21 | 442 | 1223 |
| Probe | TET-5′-catcagtatggatagaaaaccacctgccc | 31 | 465 | 1224 |
| | tg-3′-TAMRA | | | |
| Reverse | 5′-ctcgggacataagcactgca-3′ | 20 | 498 | 1225 |

TABLE VB
Panel 1.3D
Column A - Rel. Exp. (%) Ag379, Run 153691589
Column B - Rel. Exp. (%) Ag379, Run 153789843

| Tissue Name | A | B | Tissue Name | A | B |
|---|---|---|---|---|---|
| Liver adenocarcinoma | 0.0 | 4.6 | Kidney (fetal) | 1.4 | 0.0 |
| Pancreas | 0.0 | 0.0 | Renal ca. 786-0 | 0.0 | 3.3 |
| Pancreatic ca. CAPAN 2 | 0.0 | 0.0 | Renal ca. A498 | 6.3 | 0.0 |
| Adrenal gland | 0.0 | 0.0 | Renal ca. RXF 393 | 4.3 | 0.0 |
| Thyroid | 4.5 | 0.0 | Renal ca. ACHN | 4.8 | 0.0 |
| Salivary gland | 0.0 | 0.0 | Renal ca. UO-31 | 2.0 | 0.0 |
| Pituitary gland | 0.0 | 0.0 | Renal ca. TK-10 | 0.0 | 3.2 |
| Brain (fetal) | 2.4 | 0.0 | Liver | 0.0 | 3.1 |
| Brain (whole) | 2.3 | 7.4 | Liver (fetal) | 0.0 | 0.0 |
| Brain (amygdala) | 0.0 | 0.0 | Liver ca. (hepatoblast) HepG2 | 0.0 | 0.0 |
| Brain (cerebellum) | 2.1 | 0.0 | Lung | 1.9 | 3.3 |
| Brain (hippocampus) | 2.4 | 2.2 | Lung (fetal) | 0.0 | 0.0 |
| Brain (substantia nigra) | 0.0 | 0.0 | Lung ca. (small cell) LX-1 | 2.6 | 0.0 |
| Brain (thalamus) | 0.0 | 2.1 | Lung ca. (small cell) NCI-H69 | 0.0 | 0.0 |
| Cerebral Cortex | 2.2 | 28.1 | Lung ca. (s. cell var.) SHP-77 | 2.2 | 0.0 |
| Spinal cord | 3.1 | 3.1 | Lung ca. (large cell) NCI-H460 | 2.2 | 5.0 |
| glio/astro U87-MG | 0.0 | 3.5 | Lung ca. (non-sm. cell) A549 | 2.3 | 3.1 |
| glio/astro U-118-MG | 0.8 | 9.6 | Lung ca. (non-s. cell) NCI-H23 | 0.0 | 0.0 |
| astrocytoma SW1783 | 2.0 | 0.0 | Lung ca. (non-s. cell) HOP-62 | 28.1 | 2.8 |
| neuro*; met SK-N-AS | 4.5 | 0.0 | Lung ca. (non-s. cl) NCI-H522 | 7.1 | 0.0 |
| astrocytoma SF-539 | 4.5 | 18.4 | Lung ca. (squam.) SW 900 | 1.9 | 0.0 |
| astrocytoma SNB-75 | 9.2 | 0.0 | Lung ca. (squam.) NCI-H596 | 0.0 | 0.0 |
| glioma SNB-19 | 6.0 | 15.4 | Mammary gland | 0.0 | 0.0 |
| glioma U251 | 4.0 | 3.5 | Breast ca.* (pl. ef) MCF-7 | 8.0 | 2.1 |
| glioma SF-295 | 5.4 | 0.0 | Breast ca.* (pl. ef) MDA-MB- | 2.4 | 7.9 |
| | | | 231 | | |
| Heart (Fetal) | 9.0 | 0.0 | Breast ca.* (pl. ef) T47D | 0.0 | 0.0 |
| Heart | 0.0 | 0.0 | Breast ca. BT-549 | 0.0 | 0.0 |
| Skeletal muscle (Fetal) | 0.0 | 3.4 | Breast ca. MDA-N | 2.3 | 0.0 |
| Skeletal muscle | 2.9 | 2.4 | Ovary | 2.6 | 0.0 |
| Bone marrow | 5.9 | 0.0 | Ovarian ca. OVCAR-3 | 0.0 | 0.0 |
| Thymus | 0.0 | 0.0 | Ovarian ca. OVCAR-4 | 0.0 | 0.0 |
| Spleen | 0.0 | 0.0 | Ovarian ca. OVCAR-5 | 4.9 | 0.0 |
| Lymph node | 1.7 | 4.0 | Ovarian ca. OVCAR-8 | 7.1 | 6.4 |
| Colorectal | 8.8 | 6.8 | Ovarian ca. IGROV-1 | 2.2 | 0.0 |
| Stomach | 0.0 | 0.0 | Ovarian ca. (ascites) SK-OV-3 | 0.0 | 5.8 |
| Small intestine | 0.0 | 0.0 | Uterus | 1.8 | 0.0 |
| Colon ca. SW480 | 0.0 | 4.2 | Placenta | 0.0 | 0.0 |
| Colon ca.* SW620 (SW480 met) | 4.5 | 0.0 | Prostate | 2.0 | 3.7 |
| Colon ca. HT29 | 4.9 | 0.0 | Prostate ca.* (bone met) PC-3 | 3.6 | 3.2 |
| Colon ca. HCT-116 | 2.8 | 0.0 | Testis | 100.0 | 100.0 |
| Colon ca. CaCo-2 | 0.0 | 0.0 | Melanoma Hs688(A).T | 0.0 | 3.4 |
| CC Well to Mod Diff | 0.0 | 0.0 | Melanoma* (met) Hs688(B).T | 0.0 | 0.0 |
| (ODO3866) | | | | | |
| Colon ca. HCC-2998 | 2.3 | 0.0 | Melanoma UACC-62 | 2.1 | 0.0 |
| Gastric ca. (liver met) NCI-N87 | 0.0 | 0.0 | Melanoma M14 | 0.0 | 4.1 |
| Bladder | 0.0 | 4.7 | Melanoma LOX IMVI | 2.5 | 0.0 |
| Trachea | 6.6 | 0.0 | Melanoma* (met) SK-MEL-5 | 0.0 | 0.0 |
| Kidney | 0.0 | 0.0 | Adipose | 2.0 | 0.0 |

TABLE VC
Panel 4.1D
Column A - Rel. Exp.(%) Ag379, Run 169827850

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 5.3 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 16.6 | Microvascular Dermal EC none | 0.0 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 4.5 |
| Secondary CD8 lymphocyte act | 6.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNF alpha + IL- | 5.0 |
| | | 1beta | |
| LAK cells IL-2 | 0.0 | Liver cirrhosis | 13.2 |
| LAK cells IL-2 + IL-12 | 0.0 | NCI-H292 none | 97.3 |
| LAK cells IL-2 + IFN gamma | 0.0 | NCI-H292 IL-4 | 50.0 |
| LAK cells IL-2 + IL-18 | 0.0 | NCI-H292 IL-9 | 47.0 |
| LAK cells PMA/ionomycin | 0.0 | NCI-H292 IL-13 | 56.6 |
| NK Cells IL-2 rest | 0.0 | NCI-H292 IFN gamma | 100.0 |
| Two Way MLR 3 day | 0.0 | HPAEC none | 0.0 |
| Two Way MLR 5 day | 9.3 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + IL-1beta | 0.0 |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and IL-4 | 0.0 | Dermal fibroblast CCD1070 TNF alpha | 0.0 |
| EOL-1 dbcAMP | 0.0 | Dermal fibroblast CCD1070 IL-1beta | 11.5 |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 6.2 | Lung | 0.0 |
| Macrophages LPS | 0.0 | Thymus | 8.0 |
| HUVEC none | 0.0 | Kidney | 0.0 |
| HUVEC starved | 0.0 | | |

Panel 1.3D Summary: Ag379 Highest expression of this gene was detected in testis (CTs=32-33). Therapeutic modulation of this gene, expressed protein and/or use of antibodies or small molecule drugs targeting the gene or gene product is useful in the treatment of disorders of the testis, such as infertility.

Panel 4.1D Summary: Ag379 Highest expression of this gene was detected in NCI-H292 cells stimulated by IFN-gamma (CT=34). The gene was also expressed in untreated samples from the NCI-H292 cell line, a human airway epithelial cell line that produces mucins. Mucus overproduction is an important feature of bronchial asthma and chronic obstructive pulmonary disease samples. The expression of the transcript in a mucoepidermoid cell line that is often used as a model for airway epithelium (NCI-H292 cells) showed that this transcript may be important in the proliferation or activation of airway epithelium. Therapeutic modulation of this gene, expressed protein and/or use of antibodies or small molecule drugs targeting the gene or gene product is useful in the treatment of inflammation in lung epithelia in chronic obstructive pulmonary disease, asthma, allergy, and emphysema.

W. CG53746-04: Odorant Receptor S25

Expression of full-length physical clone CG53746-04 was assessed using the primer-probe set Ag2690, described in Table WA. Results of the RTQ-PCR runs are shown in Tables WB and WC.

TABLE WA
Probe Name Ag2690

| | | | Start | SEQ ID |
|---|---|---|---|---|
| Primers | Sequences | Length | Position | No |
| Forward | 5′-agaacaaggtggtgtctgtgtt-3′ | 22 | 1456 | 1226 |
| Probe | TET-5′-ctacaccgtggtgattcccatgttga-3′- | 26 | 1478 | 1227 |
| | TAMRA | | | |
| Reverse | 5′-cttgttcctgaggctgtagatc-3′ | 22 | 1511 | 1228 |

TABLE WB
Panel 2D
Column A - Rel. Exp.(%) Ag2690, Run 153131447

| Tissue Name | A | Tissue Name | A |
|---|---|---|---|
| Normal Colon | 8.4 | Kidney Margin 8120608 | 0.0 |
| CC Well to Mod Diff (ODO3866) | 0.0 | Kidney Cancer 8120613 | 0.0 |
| CC Margin (ODO3866) | 0.0 | Kidney Margin 8120614 | 1.9 |
| CC Gr.2 rectosigmoid (ODO3868) | 0.0 | Kidney Cancer 9010320 | 0.0 |
| CC Margin (ODO3868) | 0.2 | Kidney Margin 9010321 | 0.6 |
| CC Mod Diff (ODO3920) | 0.0 | Normal Uterus | 0.0 |
| CC Margin (ODO3920) | 1.1 | Uterine Cancer 064011 | 3.4 |
| CC Gr.2 ascend colon (ODO3921) | 0.0 | Normal Thyroid | 0.0 |
| CC Margin (ODO3921) | 0.0 | Thyroid Cancer | 0.8 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 2.6 | Normal Breast | 66.9 |
| Lung Margin (OD04451-02) | 10.0 | Breast Cancer | 0.0 |
| Normal Prostate 6546-1 | 10.0 | Breast Cancer (OD04590-01) | 0.7 |
| Prostate Cancer (OD04410) | 4.2 | Breast Cancer Mets (OD04590-03) | 0.0 |
| Prostate Margin (OD04410) | 13.7 | Breast Cancer Metastasis | 0.0 |
| Prostate Cancer (OD04720-01) | 62.4 | Breast Cancer | 10.1 |
| Prostate Margin (OD04720-02) | 27.4 | Breast Cancer | 85.3 |
| Normal Lung | 100.0 | Breast Cancer 9100266 | 13.3 |
| Lung Met to Muscle (ODO4286) | 0.5 | Breast Margin 9100265 | 22.2 |
| Muscle Margin (ODO4286) | 0.0 | Breast Cancer A209073 | 51.4 |
| Lung Malignant Cancer (OD03126) | 5.9 | Breast Margin A209073 | 78.5 |
| Lung Margin (OD03126) | 39.8 | Normal Liver | 0.0 |
| Lung Cancer (OD04404) | 4.0 | Liver Cancer | 2.0 |
| Lung Margin (OD04404) | 17.9 | Liver Cancer 1025 | 0.1 |
| Lung Cancer (OD04565) | 1.1 | Liver Cancer 1026 | 0.0 |
| Lung Margin (OD04565) | 19.8 | Liver Cancer 6004-T | 0.5 |
| Lung Cancer (OD04237-01) | 0.0 | Liver Tissue 6004-N | 0.9 |
| Lung Margin (OD04237-02) | 25.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 4.1 |
| Melanoma Metastasis | 0.0 | Bladder Cancer | 0.0 |
| Lung Margin (OD04321) | 47.0 | Bladder Cancer | 4.0 |
| Normal Kidney | 6.0 | Bladder Cancer (OD04718-01) | 9.9 |
| Kidney Ca, Nuclear grade 2 (OD04338) | 2.9 | Bladder Normal Adjacent | 0.6 |
| | | (OD04718-03) | |
| Kidney Margin (OD04338) | 8.6 | Normal Ovary | 0.0 |
| Kidney Ca Nuclear grade ½ (OD04339) | 0.0 | Ovarian Cancer | 0.6 |
| Kidney Margin (OD04339) | 2.3 | Ovarian Cancer (OD04768-07) | 4.7 |
| Kidney Ca, Clear cell type (OD04340) | 0.0 | Ovary Margin (OD04768-08) | 0.5 |
| Kidney Margin (OD04340) | 0.5 | Normal Stomach | 1.3 |
| Kidney Ca, Nuclear grade 3 (OD04348) | 0.0 | Gastric Cancer 9060358 | 0.0 |
| Kidney Margin (OD04348) | 2.6 | Stomach Margin 9060359 | 2.5 |
| Kidney Cancer (OD04622-01) | 0.6 | Gastric Cancer 9060395 | 0.0 |
| Kidney Margin (OD04622-03) | 0.8 | Stomach Margin 9060394 | 1.7 |
| Kidney Cancer (OD04450-01) | 0.0 | Gastric Cancer 9060397 | 0.0 |
| Kidney Margin (OD04450-03) | 2.8 | Stomach Margin 9060396 | 2.2 |
| Kidney Cancer 8120607 | 0.0 | Gastric Cancer 064005 | 2.4 |

TABLE WC
Panel 4D
Column A - Rel. Exp.(%) Ag2690, Run 153131451

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.1 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.2 |
| | | 1beta | |
| Primary Th2 act | 0.0 | Microvascular Dermal EC none | 0.3 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.1 |
| Primary Th2 rest | 0.0 | Small airway epithelium none | 6.4 |
| Primary Tr1 rest | 0.0 | Small airway epithelium TNF alpha + IL- | 22.1 |
| | | 1beta | |
| CD45RA CD4 lymphocyte act | 0.2 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 3.5 |
| Secondary CD8 lymphocyte rest | 0.0 | Astrocytes TNF alpha + IL-1beta | 1.8 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 4.4 |
| CH11 | | | |
| LAK cells rest | 0.0 | CCD1106 (Keratinocytes) TNF alpha + IL- | 0.0 |
| | | 1beta | |
| LAK cells IL-2 | 0.0 | Liver cirrhosis | 1.1 |
| LAK cells IL-2 + IL-12 | 0.0 | Lupus kidney | 1.1 |
| LAK cells IL-2 + IFN gamma | 0.0 | NCI-H292 none | 42.9 |
| LAK cells IL-2 + IL-18 | 0.0 | NCI-H292 IL-4 | 100.0 |
| LAK cells PMA/ionomycin | 0.0 | NCI-H292 IL-9 | 41.8 |
| NK Cells IL-2 rest | 0.0 | NCI-H292 IL-13 | 37.4 |
| Two Way MLR 3 day | 0.0 | NCI-H292 IFN gamma | 15.7 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.3 |
| PBMC PWM | 0.0 | Lung fibroblast TNF alpha + IL-1beta | 0.0 |
| PBMC PHA-L | 0.0 | Lung fibroblast IL-4 | 0.0 |
| Ramos (B cell) none | 0.0 | Lung fibroblast IL-9 | 0.3 |
| Ramos (B cell) ionomycin | 0.0 | Lung fibroblast IL-13 | 0.4 |
| B lymphocytes PWM | 0.2 | Lung fibroblast IFN gamma | 0.6 |
| B lymphocytes CD40L and IL-4 | 0.0 | Dermal fibroblast CCD1070 rest | 0.6 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.5 |
| Monocytes rest | 0.0 | IBD Crohn's | 0.1 |
| Monocytes LPS | 0.0 | Colon | 0.0 |
| Macrophages rest | 0.0 | Lung | 4.5 |
| Macrophages LPS | 0.0 | Thymus | 2.0 |
| HUVEC none | 0.0 | Kidney | 1.5 |
| HUVEC starved | 0.0 | | |

Panel 2D Summary: Ag2690 Highest expression of this gene was seen in normal lung tissue (CT=30)/. In addition, this gene was overexpressed in normal lung tissue when compared to expression in adjacent malignant tissue. Thus, expression of this gene is useful as a marker of lung cancer. Therapeutic modulation of this gene, expressed protein and/or use of antibodies or small molecule drugs targeting the gene or gene product is useful in the treatment of lung cancer.

Panel 4D Summary: Ag2690 Highest expression of this gene was seen in IL-4 treated NCI-H292 cells, a human airway epithelial cell line that produces mucins (CT=28). This gene was also expressed in a cluster of treated and untreated samples derived from the NCI-H292 cell line. Mucus overproduction is an important feature of bronchial asthma and chronic obstructive pulmonary disease samples. The transcript was also expressed at lower but still significant levels in small airway epithelium treated with IL-1 beta and TNF-alpha. The expression of the transcript in this mucoepidermoid cell line that is often used as a model for airway epithelium (NCI-H292 cells) showed that this transcript is important in the proliferation or activation of airway epithelium. Therapeutic modulation of this gene, expressed protein and/or use of antibodies or small molecule drugs targeting the gene or gene product is useful in the treatment of inflammation in lung epithelia in chronic obstructive pulmonary disease, asthma, allergy, and emphysema.

X. CG53767-02: Olfactory Receptor

Expression of full-length physical clone CG53767-02 was assessed using the primer-probe sets Ag2687 and Ag440, described in Tables XA and XB. Results of the RTQ-PCR runs are shown in Table XC.

TABLE XA
Probe Name Ag2687

| | | | Start | SEQ ID |
|---|---|---|---|---|
| Primers | Sequences | Length | Position | No |
| Forward | 5′-tgtgcatcatcgtcactgtatt-3′ | 22 | 807 | 1229 |
| Probe | TET-5′-tctttgcgacttgaacactatccagca- | 27 | 838 | 1230 |
| | 3′-TAMRA | | | |
| Reverse | 5′-ggagaccactggtgagatatca-3′ | 22 | 874 | 1231 |

TABLE XB
Probe Name Ag440

| | | | Start | SEQ ID |
|---|---|---|---|---|
| Primers | Sequences | Length | Position | No |
| Forward | 5′-tgctcctcccacctcactg-3′ | 19 | 1058 | 1232 |
| Probe | TET-5′-tgtccatacactatggatttgcttgctttg | 34 | 1080 | 1233 |
| | tcta-3′-TAMRA | | | |
| Reverse | 5′-tgctgttcttgggcctcaa-3′ | 19 | 1115 | 1234 |

TABLE XC
Panel 4D
Column A - Rel. Exp.(%) Ag2687, Run 153112357

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 0.0 | Microvascular Dermal EC none | 0.2 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.5 |
| Primary Th2 rest | 0.0 | Small airway epithelium none | 2.2 |
| Primary Tr1 rest | 0.0 | Small airway epithelium TNF alpha + IL- | 17.7 |
| | | 1beta | |
| CD45RA CD4 lymphocyte act | 0.0 | Coronery artery SMC rest | 0.2 |
| CD45RO CD4 lymphocyte act | 0.0 | Coronery artery SMC TNF alpha + IL-1beta | 0.0 |
| CD8 lymphocyte act | 0.0 | Astrocytes rest | 1.4 |
| Secondary CD8 lymphocyte rest | 0.0 | Astrocytes TNF alpha + IL-1beta | 1.3 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.2 |
| 2ry Th1/Th2/Tr1 anti-CD95 | 0.0 | CCD1106 (Keratinocytes) none | 1.7 |
| CH11 | | | |
| LAK cells rest | 0.0 | CCD1106 (Keratinocytes) TNF alpha + IL- | 0.0 |
| | | 1beta | |
| LAK cells IL-2 | 0.0 | Liver cirrhosis | 1.2 |
| LAK cells IL-2 + IL-12 | 0.0 | Lupus kidney | 0.7 |
| LAK cells IL-2 + IFN gamma | 0.0 | NCI-H292 none | 54.7 |
| LAK cells IL-2 + IL-18 | 0.0 | NCI-H292 IL-4 | 100.0 |
| LAK cells PMA/ionomycin | 0.0 | NCI-H292 IL-9 | 40.1 |
| NK Cells IL-2 rest | 0.0 | NCI-H292 IL-13 | 38.2 |
| Two Way MLR 3 day | 0.0 | NCI-H292 IFN gamma | 9.9 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.4 |
| PBMC PWM | 0.0 | Lung fibroblast TNF alpha + IL-1beta | 0.0 |
| PBMC PHA-L | 0.0 | Lung fibroblast IL-4 | 0.6 |
| Ramos (B cell) none | 0.2 | Lung fibroblast IL-9 | 0.4 |
| Ramos (B cell) ionomycin | 0.0 | Lung fibroblast IL-13 | 0.3 |
| B lymphocytes PWM | 0.4 | Lung fibroblast IFN gamma | 0.0 |
| B lymphocytes CD40L and IL-4 | 0.0 | Dermal fibroblast CCD1070 rest | 0.4 |
| EOL-1 dbcAMP | 0.0 | Dermal fibroblast CCD1070 TNF alpha | 0.4 |
| EOL-1 dbcAMP | 0.0 | Dermal fibroblast CCD1070 IL-1beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.4 |
| Dendritic cells anti-CD40 | 0.0 | IBD Colitis 2 | 0.0 |
| Monocytes rest | 0.1 | IBD Crohn's | 0.0 |
| Monocytes LPS | 0.0 | Colon | 0.4 |
| Macrophages rest | 0.0 | Lung | 3.6 |
| Macrophages LPS | 0.0 | Thymus | 10.9 |
| HUVEC none | 0.0 | Kidney | 0.8 |
| HUVEC starved | 0.0 | | |

Panel 4D Summary: Ag2687 Highest expression of this gene was seen in IL-4 treated NCI-H292 cells, a human airway epithelial cell line that produces mucins (CT=28.8). The gene was also expressed in a cluster of treated and untreated samples derived from the NCI-H292 cell line. Mucus overproduction is an important feature of bronchial asthma and chronic obstructive pulmonary disease samples. The transcript was also expressed at lower but significant levels in small airway epithelium treated with IL-1 beta and TNF-alpha. CG53767-02 gene expression in a mucoepidermoid cell line that is often used as a model for airway epithelium (NCI-H292 cells) showed that this gene is important in the proliferation or activation of airway epithelium. Therapeutic modulation of the activity of this gene or its protein product using nucleic acid, protein, antibody or small molecule drugs is useful in reducing or eliminating the symptoms caused by inflammation in lung epithelia in chronic obstructive pulmonary disease, asthma, allergy, and emphysema.

Y. CG53776-02: Olfactory Receptor

Expression of full-length physical clone CG53776-02 was assessed using the primer-probe set Ag7081, described in Table YA. Results of the RTQ-PCR runs are shown in Table YB.

TABLE YA
Probe Name Ag7081

| | | | Start | SEQ ID |
|---|---|---|---|---|
| Primers | Sequences | Length | Position | No |
| Forward | 5′-atctacctggtaaccatatctggtaat-3′ | 27 | 168 | 1235 |
| Probe | TET-5′-ttcttatcagaatttcttctcagctcca- | 28 | 208 | 1236 |
| | 3′-TAMRA | | | |
| Reverse | 5′-gctcagaaagaaatacataggatga-3′ | 25 | 236 | 1237 |

TABLE YB
General_screening_panel_v1.6
Column A - Rel. Exp.(%) Ag7081, Run 283147418

| Tissue Name | A | Tissue Name | A |
|---|---|---|---|
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous Cell | 0.0 | Colon ca.* (SW480 met) SW620 | 0.0 |
| carcinoma SCC-4 | | | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 2.3 | Colon ca. CaCo-2 | 3.6 |
| Placenta | 11.2 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 3.6 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 3.3 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 2.5 |
| Trachea | 3.6 | CNS cancer (glio/astro) U87-MG | 0.0 |
| Lung | 0.0 | CNS cancer (glio/astro) U-118-MG | 3.0 |
| Fetal Lung | 100.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

General_screening_panel_v1.6 Summary: Ag7081 Low but significant expression of this gene was detected in fetal lung (CT=34.3). The relative overexpression of this gene in fetal lung showed that the protein enhances lung growth or development in the fetus and also acts in a regenerative capacity in the adult.

Z. CG53803-02: Olfactory Receptor

Expression of full-length physical clone CG53803-02 was assessed using the primer-probe set Ag2018, described in Table ZA. Results of the RTQ-PCR runs are shown in Tables ZB and ZC.

TABLE ZA
Probe Name Ag2018

| | | | Start | SEQ ID |
|---|---|---|---|---|
| Primers | Sequences | Length | Position | No |
| Forward | 5′-atgctctccggttaaacttctc-3′ | 22 | 544 | 1238 |
| Probe | TET-5′-tggacctaatgtaatcaaccacttcttt | 30 | 566 | 1239 |
| | tg-3′-TAMRA | | | |
| Reverse | 5′-agagccagacacagagatgaga-3′ | 22 | 608 | 1240 |

TABLE ZB
Panel 2D
Column A - Rel. Exp.(%) Ag2018, Run 152517542

| Tissue Name | A | Tissue Name | A |
|---|---|---|---|
| Normal Colon | 0.0 | Kidney Margin 8120608 | 0.0 |
| CC Well to Mod Diff (ODO3866) | 0.0 | Kidney Cancer 8120613 | 0.0 |
| CC Margin (ODO3866) | 0.8 | Kidney Margin 8120614 | 0.0 |
| CC Gr.2 rectosigmoid (ODO3868) | 0.0 | Kidney Cancer 9010320 | 0.0 |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterine Cancer 064011 | 0.0 |
| CC Gr.2 ascend colon (ODO3921) | 0.0 | Normal Thyroid | 0.0 |
| CC Margin (ODO3921) | 0.2 | Thyroid Cancer | 0.0 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 0.0 | Normal Breast | 0.0 |
| Lung Margin (OD04451-02) | 0.0 | Breast Cancer | 0.0 |
| Normal Prostate 6546-1 | 0.0 | Breast Cancer (OD04590-01) | 0.0 |
| Prostate Cancer (OD04410) | 0.0 | Breast Cancer Mets (OD04590-03) | 0.0 |
| Prostate Margin (OD04410) | 0.0 | Breast Cancer Metastasis | 0.0 |
| Prostate Cancer (OD04720-01) | 0.0 | Breast Cancer | 0.0 |
| Prostate Margin (OD04720-02) | 0.0 | Breast Cancer | 0.0 |
| Normal Lung | 0.0 | Breast Cancer 9100266 | 0.0 |
| Lung Met to Muscle (ODO4286) | 0.1 | Breast Margin 9100265 | 100.0 |
| Muscle Margin (ODO4286) | 0.0 | Breast Cancer A209073 | 0.2 |
| Lung Malignant Cancer (OD03126) | 0.0 | Breast Margin A209073 | 0.0 |
| Lung Margin (OD03126) | 0.0 | Normal Liver | 0.0 |
| Lung Cancer (OD04404) | 0.0 | Liver Cancer | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 0.0 |
| Lung Margin (OD04237-02) | 0.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 0.0 |
| Melanoma Metastasis | 0.0 | Bladder Cancer | 0.0 |
| Lung Margin (OD04321) | 0.0 | Bladder Cancer | 0.8 |
| Normal Kidney | 0.0 | Bladder Cancer (OD04718-01) | 0.0 |
| Kidney Ca, Nuclear grade 2 (OD04338) | 0.0 | Bladder Normal Adjacent | 0.0 |
| | | (OD04718-03) | |
| Kidney Margin (OD04338) | 0.0 | Normal Ovary | 0.0 |
| Kidney Ca Nuclear grade ½ (OD04339) | 0.0 | Ovarian Cancer | 0.0 |
| Kidney Margin (OD04339) | 0.0 | Ovarian Cancer (OD04768-07) | 0.0 |
| Kidney Ca, Clear cell type (OD04340) | 0.0 | Ovary Margin (OD04768-08) | 0.0 |
| Kidney Margin (OD04340) | 0.0 | Normal Stomach | 0.0 |
| Kidney Ca, Nuclear grade 3 (OD04348) | 0.0 | Gastric Cancer 9060358 | 0.2 |
| Kidney Margin (OD04348) | 0.0 | Stomach Margin 9060359 | 0.0 |
| Kidney Cancer (OD04622-01) | 0.0 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 0.0 |
| Kidney Margin (OD04450-03) | 0.0 | Stomach Margin 9060396 | 0.0 |
| Kidney Cancer 8120607 | 0.0 | Gastric Cancer 064005 | 0.0 |

TABLE ZC
Panel 4D
Column A - Rel. Exp.(%) Ag2018, Run 152784446

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 1.4 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 0.0 | Microvascular Dermal EC none | 0.0 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNF alpha + IL- | 0.0 |
| | | 1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL-1beta | 0.0 |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 4.5 | Dermal fibroblast CCD1070 rest | 2.1 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 31.4 |
| Monocytes rest | 0.0 | IBD Crohn's | 3.8 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 2D Summary: Ag2018 Significant expression of this gene was seen in a single normal breast sample (CT=27.6). Expression was down-regulated in the matched adjacent breast tumor tissue (CT=40). Therapeutic modulation of this gene, expressed protein and/or use of antibodies or small molecule drugs targeting the gene or gene product is useful in the treatment of breast cancer.

Panel 4D Summary: Ag2018 Significant expression of this gene was detected in a liver cirrhosis sample (CT=33.7). Epression of this gene was not detected in normal liver in Panel 1.3D, demonstrating that expression of this gene is unique to liver cirrhosis. This gene encodes a GPCR; therefore, antibodies or small molecule therapeutics will reduce or inhibit fibrosis that occurs in liver cirrhosis. In addition, antibodies to this GPCR are useful for the diagnosis of liver cirrhosis.

AA. CG53989-04: Mastocytoma Protease Precursor-Like.

Expression of gene CG53989-04 was assessed using the primer-probe sets Ag1038, Ag1590, Ag1918, Ag2899, Ag720, Ag730, Ag443, Ag5819, Ag6974 and Ag8406, described in Tables AAA, AAB, AAC, AAD, AAE, AAF, AAG, AAH, AAI and AAJ. Results of the RTQ-PCR runs are shown in Tables AAK, AAL, AAM, AAN and AAO.

TABLE AAA
Probe Name Ag1038

| | | | Start | SEQ ID |
|---|---|---|---|---|
| Primers | Sequences | Length | Position | No |
| Forward | 5′-aggagcaacgtcctctgtaac-3′ | 21 | 538 | 1241 |
| Probe | TET-5′-cttccaaccacactgagcggtttgag-3′- | 26 | 581 | 1242 |
| | TAMRA | | | |
| Reverse | 5′-agcatgtcgtccttgatgag-3′ | 20 | 610 | 1243 |

TABLE AAB
Probe Name Ag1590

| | | | Start | SEQ ID |
|---|---|---|---|---|
| Primers | Sequences | Length | Position | No |
| Forward | 5′-aggagcaacgtcctctgtaac-3′ | 21 | 538 | 1244 |
| Probe | TET-5′-cttccaaccacactgagcggtttgag-3′- | 26 | 581 | 1245 |
| | TAMRA | | | |
| Reverse | 5′-agcatgtcgtccttgatgag-3′ | 20 | 610 | 1246 |

TABLE AAC
Probe Name Ag1918

| | | | Start | SEQ ID |
|---|---|---|---|---|
| Primers | Sequences | Length | Position | No |
| Forward | 5′-aggagcaacgtcctctgtaac-3′ | 21 | 538 | 1247 |
| Probe | TET-5′-cttccaaccacactgagcggtttgag-3′- | 26 | 581 | 1248 |
| | TAMRA | | | |
| Reverse | 5′-agcatgtcgtccttgatgag-3′ | 20 | 610 | 1249 |

TABLE AAD
Probe Name Ag2899

| | | | Start | SEQ ID |
|---|---|---|---|---|
| Primers | Sequences | Length | Position | No |
| Forward | 5′-aggagcaacgtcctctgtaac-3′ | 21 | 538 | 1250 |
| Probe | TET-5′-cttccaaccacactgagcggtttgag-3′- | 26 | 581 | 1251 |
| | TAMRA | | | |
| Reverse | 5′-agcatgtcgtccttgatgag-3′ | 20 | 610 | 1252 |

TABLE AAE
Probe Name Ag720

| | | | Start | SEQ ID |
|---|---|---|---|---|
| Primers | Sequences | Length | Position | No |
| Forward | 5′-aggagcaacgtcctctgtaac-3′ | 21 | 538 | 1253 |
| Probe | TET-5′-cttccaaccacactgagcggtttgag-3′- | 26 | 581 | 1254 |
| | TAMRA | | | |
| Reverse | 5′-acagcatgtcgtccttgatg-3′ | 20 | 612 | 1255 |

TABLE AAF
Probe Name Ag730

| | | | Start | SEQ ID |
|---|---|---|---|---|
| Primers | Sequences | Length | Position | No |
| Forward | 5′-caggagcaacgtcctctgta-3′ | 20 | 537 | 1256 |
| Probe | TET-5′-cttccaaccacactgagcggtttgag-3′- | 26 | 581 | 1257 |
| | TAMRA | | | |
| Reverse | 5′-cacacagcatgtcgtcctt-3′ | 19 | 616 | 1258 |

TABLE AAG
Probe Name Ag443

| | | | Start | SEQ ID |
|---|---|---|---|---|
| Primers | Sequences | Length | Position | No |
| Forward | 5′-gacggtgaaggtcaggagca-3′ | 20 | 525 | 1259 |
| Probe | TET-5′-cctgtcgccgccgctttcc-3′-TAMRA | 19 | 563 | 1260 |
| Reverse | 5′-aaccgctcagtgtggttgg-3′ | 19 | 584 | 1261 |

TABLE AAH
Probe Name Ag5819

| | | | Start | SEQ ID |
|---|---|---|---|---|
| Primers | Sequences | Length | Position | No |
| Forward | 5′-cctggtagggaggagttgga-3′ | 20 | 100 | 1262 |
| Probe | TET-5′-ctgaggctctatgaggacgaccagcggac- | 29 | 151 | 1263 |
| | 3′-TAMRA | | | |
| Reverse | 5′-ccaggcccagcaggtcttc-3′ | 19 | 339 | 1264 |

TABLE AAI
Probe Name Ag6974

| | | | Start | SEQ ID |
|---|---|---|---|---|
| Primers | Sequences | Length | Position | No |
| Forward | 5′-gagacggatggccacag-3′ | 17 | 395 | 1265 |
| Probe | TET-5′-ccaggtggctcagagcagcaggaatgtac- | 29 | 413 | 1266 |
| | 3′-TAMRA | | | |
| Reverse | 5′-cgttccgcctgcagag-3′ | 16 | 452 | 1267 |

TABLE AAJ
Probe Name Ag8406

| | | | Start | SEQ ID |
|---|---|---|---|---|
| Primers | Sequences | Length | Position | No |
| Forward | 5′-gtgagctggtgggcatc-3′ | 17 | 49 | 1268 |
| Probe | TET-5′-ctccctaccaggggtgcctcctg-3′- | 23 | 89 | 1269 |
| | TAMRA | | | |
| Reverse | 5′-cactctaaacgcgcaagc-3′ | 18 | 121 | 1270 |

TABLE AAK
Ardais Breast1.0
Column A - Rel. Exp.(%) Ag720, Run 389241931

| Tissue Name | A | Tissue Name | A |
|---|---|---|---|
| 111297 Breast cancer metastasis (9369)* | 0.0 | 153636 Breast cancer (D3D) | 0.4 |
| 108830 Breast cancer metastasis | 1.1 | 164668 Breast cancer (6314) | 0.1 |
| (OD06855)* | | | |
| 97764 Breast cancer node metastasis | 1.3 | 164677 Breast cancer (5272) | 12.4 |
| (OD06083) | | | |
| 97739 Breast cancer (CHTN20676) | 0.1 | 164685 Breast cancer (0170) | 11.3 |
| 145848 Breast cancer (9B6) | 8.4 | 98857 Breast cancer (OD06397- | 0.0 |
| | | 12) | |
| 145859 Breast cancer (9EC) | 1.2 | 153628 Breast cancer (D35) | 0.8 |
| 153632 Breast cancer (D39) | 0.0 | 153637 Breast cancer (D3E) | 5.4 |
| 153643 Breast cancer (D44) | 10.7 | 164669 Breast cancer (6992) | 6.0 |
| 164672 Breast cancer (7464) | 26.1 | 164678 Breast cancer (5297) | 68.8 |
| 164681 Breast cancer (5787) | 9.7 | 164686 Breast cancer (0732) | 0.5 |
| 97748 Breast cancer (CHTN20931) | 2.2 | 145857 Breast cancer (9F0) | 15.2 |
| 145850 Breast cancer (9C7) | 0.1 | 153630 Breast cancer (D37) | 0.6 |
| 149844 Breast cancer (24178) | 2.5 | 153638 Breast cancer (D3F) | 23.7 |
| 153633 Breast cancer (D3A) | 0.4 | 164670 Breast cancer (7078) | 1.1 |
| 153644 Breast cancer (D45) | 9.0 | 164679 Breast cancer (5486) | 4.3 |
| 164673 Breast cancer (8452) | 29.1 | 164687 Breast cancer (5881) | 0.5 |
| 164682 Breast cancer (6342)* | 7.7 | 145846 Breast cancer (9B7) | 22.5 |
| 97751 Breast cancer (CHTN21053) | 0.8 | 145858 Breast cancer (9B4) | 0.0 |
| 116417 Breast cancer (3367)* | 0.1 | 153631 Breast cancer (D38) | 1.6 |
| 145852 Breast cancer (A1A) | 13.0 | 153639 Breast cancer (D40) | 22.2 |
| 151097 Breast cancer (CHTN24298) | 0.4 | 164671 Breast cancer (7082) | 0.3 |
| 153634 Breast cancer (D3B) | 0.8 | 164680 Breast cancer (5705) | 0.0 |
| 155797 Breast cancer (EA6) | 2.2 | 164688 Breast cancer (7222) | 0.0 |
| 164674 Breast cancer (8811) | 8.2 | 111288 Breast NAT (3367) | 0.3 |
| 164683 Breast cancer (6470) | 2.4 | 111302 Breast NAT (6314) | 0.6 |
| 97763 Breast cancer (OD06083) | 0.8 | 105687 Breast cancer 1B | 0.0 |
| 116418 Breast cancer (3378)* | 1.3 | 105688 Breast NAT 1A | 0.0 |
| 145853 Breast cancer (9F3) | 0.4 | 105689 Breast cancer 2B | 0.0 |
| 153432 Breast cancer (CHTN 24652) | 16.6 | 105690 Breast NAT 2A | 0.0 |
| 153635 Breast cancer (D3C) | 1.1 | 111289 Breast cancer 3B* | 0.0 |
| 164667 Breast cancer (5785) | 1.6 | 111290 Breast NAT 3A* | 0.0 |
| 164676 Breast cancer (5070) | 100.0 | 116424 Breast cancer 4B* | 0.1 |
| 164684 Breast cancer (6509) | 24.8 | 116425 Breast NAT 4A | 0.0 |
| 116421 Breast cancer (6314) | 0.0 | 108847 Breast cancer | 0.0 |
| 145854 Breast cancer (9B8) | 5.9 | 105694 Breast NAT | 0.0 |
| 153627 Breast cancer (D34) | 0.2 | | |

TABLE AAL
Panel 1.3D
Column A - Rel. Exp.(%) Ag1590, Run 152059684
Column B - Rel. Exp.(%) Ag1590, Run 155330156
Column C - Rel. Exp.(%) Ag2899, Run 160943164
Column D - Rel. Exp.(%) Ag2899, Run 165518181

| Tissue Name | A | B | C | D |
|---|---|---|---|---|
| Liver adenocarcinoma | 0.0 | 1.1 | 1.7 | 0.0 |
| Pancreas | 0.0 | 0.0 | 0.0 | 6.9 |
| Pancreatic ca. CAPAN 2 | 8.7 | 2.5 | 5.6 | 10.5 |
| Adrenal gland | 0.0 | 0.8 | 0.0 | 0.0 |
| Thyroid | 0.0 | 0.0 | 0.0 | 0.0 |
| Salivary gland | 0.0 | 0.0 | 0.0 | 2.4 |
| Pituitary gland | 34.2 | 34.2 | 16.7 | 42.0 |
| Brain (fetal) | 0.0 | 0.0 | 0.0 | 1.9 |
| Brain (whole) | 0.0 | 0.0 | 0.0 | 0.0 |
| Brain (amygdala) | 0.0 | 0.0 | 0.0 | 0.0 |
| Brain (cerebellum) | 0.0 | 0.0 | 0.0 | 0.0 |
| Brain (hippocampus) | 0.0 | 0.0 | 0.0 | 0.0 |
| Brain (substantia nigra) | 0.0 | 0.0 | 0.0 | 0.0 |
| Brain (thalamus) | 0.0 | 0.0 | 0.0 | 0.0 |
| Cerebral Cortex | 1.2 | 0.0 | 0.0 | 0.0 |
| Spinal cord | 0.0 | 0.0 | 0.0 | 0.0 |
| glio/astro U87-MG | 0.0 | 0.0 | 0.0 | 0.0 |
| glio/astro U-118-MG | 0.0 | 0.0 | 0.0 | 0.0 |
| astrocytoma SW1783 | 0.3 | 1.0 | 0.0 | 2.3 |
| neuro*; met SK-N-AS | 0.0 | 0.0 | 0.0 | 0.0 |
| astrocytoma SF-539 | 0.0 | 0.0 | 0.0 | 0.0 |
| astrocytoma SNB-75 | 0.7 | 0.0 | 0.0 | 4.5 |
| glioma SNB-19 | 0.0 | 0.0 | 0.0 | 2.5 |
| glioma U251 | 0.0 | 0.0 | 0.0 | 0.0 |
| glioma SF-295 | 0.0 | 0.0 | 0.0 | 0.0 |
| Heart (Fetal) | 0.0 | 1.4 | 0.0 | 0.0 |
| Heart | 0.0 | 0.0 | 0.0 | 0.0 |
| Skeletal muscle (Fetal) | 0.0 | 0.0 | 0.0 | 0.0 |
| Skeletal muscle | 0.0 | 0.0 | 0.0 | 0.0 |
| Bone marrow | 0.9 | 0.0 | 1.0 | 0.0 |
| Thymus | 0.7 | 0.9 | 1.4 | 3.7 |
| Spleen | 0.6 | 1.7 | 0.0 | 0.0 |
| Lymph node | 0.0 | 0.0 | 0.0 | 0.0 |
| Colorectal | 3.9 | 2.4 | 1.6 | 8.8 |
| Stomach | 0.0 | 2.4 | 0.0 | 0.0 |
| Small intestine | 0.0 | 0.0 | 0.0 | 2.0 |
| Colon ca. SW480 | 0.8 | 1.5 | 0.0 | 0.0 |
| Colon ca.* SW620 (SW480 met) | 0.9 | 0.9 | 3.4 | 0.0 |
| Colon ca. HT29 | 0.0 | 0.0 | 0.0 | 0.0 |
| Colon ca. HCT-116 | 0.0 | 1.0 | 0.0 | 1.0 |
| Colon ca. CaCo-2 | 0.0 | 1.4 | 0.0 | 0.0 |
| CC Well to Mod Diff (ODO3866) | 8.8 | 8.1 | 15.2 | 26.8 |
| Colon ca. HCC-2998 | 32.3 | 29.3 | 29.9 | 22.2 |
| Gastric ca. (liver met) NCI-N87 | 32.1 | 31.6 | 36.9 | 70.7 |
| Bladder | 0.8 | 0.0 | 0.0 | 0.0 |
| Trachea | 0.8 | 1.1 | 0.0 | 0.0 |
| Kidney | 1.8 | 0.0 | 1.7 | 1.0 |
| Kidney (fetal) | 3.8 | 0.9 | 7.6 | 0.0 |
| Renal ca. 786-0 | 0.0 | 0.0 | 0.0 | 0.0 |
| Renal ca. A498 | 0.8 | 0.0 | 0.0 | 0.0 |
| Renal ca. RXF 393 | 0.0 | 0.0 | 0.0 | 3.2 |
| Renal ca. ACHN | 2.1 | 0.0 | 0.0 | 0.0 |
| Renal ca. UO-31 | 0.0 | 0.0 | 0.0 | 0.0 |
| Renal ca. TK-10 | 0.0 | 0.0 | 0.0 | 0.0 |
| Liver | 4.7 | 5.2 | 3.5 | 33.9 |
| Liver (fetal) | 9.0 | 6.0 | 3.7 | 18.3 |
| Liver ca. (hepatoblast) HepG2 | 2.4 | 1.2 | 1.8 | 0.0 |
| Lung | 0.0 | 0.0 | 3.2 | 0.0 |
| Lung (fetal) | 0.4 | 0.0 | 0.0 | 0.0 |
| Lung ca. (small cell) LX-1 | 6.9 | 13.1 | 9.5 | 10.5 |
| Lung ca. (small cell) NCI-H69 | 2.2 | 1.3 | 0.8 | 0.0 |
| Lung ca. (s. cell var.) SHP-77 | 3.3 | 1.1 | 6.5 | 6.3 |
| Lung ca. (large cell) NCI-H460 | 0.0 | 0.0 | 0.0 | 0.0 |
| Lung ca. (non-sm. cell) A549 | 0.0 | 0.0 | 0.0 | 0.0 |
| Lung ca. (non-s. cell) NCI-H23 | 0.0 | 0.0 | 0.0 | 0.0 |
| Lung ca. (non-s. cell) HOP-62 | 0.8 | 0.0 | 0.0 | 0.0 |
| Lung ca. (non-s. cl) NCI-H522 | 0.0 | 0.5 | 0.0 | 0.0 |
| Lung ca. (squam.) SW 900 | 0.8 | 0.0 | 0.0 | 0.0 |
| Lung ca. (squam.) NCI-H596 | 0.4 | 0.5 | 0.0 | 0.0 |
| Mammary gland | 2.7 | 4.2 | 0.0 | 7.9 |
| Breast ca.* (pl.ef) MCF-7 | 4.1 | 0.0 | 0.0 | 2.5 |
| Breast ca.* (pl.ef) MDA-MB-231 | 0.0 | 0.0 | 1.7 | 0.0 |
| Breast ca.* (pl.ef) T47D | 6.5 | 5.3 | 0.7 | 4.3 |
| Breast ca. BT-549 | 0.0 | 0.0 | 0.0 | 0.0 |
| Breast ca. MDA-N | 0.0 | 1.1 | 1.7 | 0.0 |
| Ovary | 0.0 | 0.0 | 0.0 | 0.0 |
| Ovarian ca. OVCAR-3 | 0.0 | 0.0 | 0.0 | 0.0 |
| Ovarian ca. OVCAR-4 | 0.0 | 0.0 | 0.0 | 0.0 |
| Ovarian ca. OVCAR-5 | 100.0 | 100.0 | 100.0 | 100.0 |
| Ovarian ca. OVCAR-8 | 2.5 | 0.0 | 0.0 | 2.5 |
| Ovarian ca. IGROV-1 | 0.0 | 0.0 | 0.0 | 0.0 |
| Ovarian ca. (ascites) SK-OV-3 | 0.0 | 0.0 | 0.0 | 0.0 |
| Uterus | 0.0 | 0.0 | 0.0 | 0.0 |
| Placenta | 9.2 | 6.9 | 8.0 | 14.0 |
| Prostate | 1.9 | 5.2 | 1.8 | 4.1 |
| Prostate ca.* (bone met) PC-3 | 0.0 | 0.0 | 0.0 | 0.0 |
| Testis | 6.3 | 9.3 | 8.9 | 8.8 |
| Melanoma Hs688(A).T | 0.0 | 0.0 | 0.0 | 0.0 |
| Melanoma* (met) Hs688(B).T | 0.0 | 0.0 | 0.0 | 0.0 |
| Melanoma UACC-62 | 0.0 | 0.0 | 0.0 | 0.0 |
| Melanoma M14 | 0.0 | 0.0 | 0.0 | 0.0 |
| Melanoma LOX IMVI | 0.0 | 0.0 | 0.0 | 0.0 |
| Melanoma* (met) SK-MEL-5 | 0.0 | 0.0 | 0.0 | 0.0 |
| Adipose | 0.0 | 0.0 | 0.0 | 0.0 |

TABLE AAM
Panel 2D
Column A - Rel. Exp. (%) Ag1590, Run 152060853
Column B - Rel. Exp. (%) Ag1590, Run 155330182
Column C - Rel. Exp. (%) Ag2899, Run 160999416
Column D - Rel. Exp. (%) Ag2899, Run 164988403
Column E - Rel. Exp. (%) Ag720, Run 145375720

| Tissue Name | A | B | C | D | E |
|---|---|---|---|---|---|
| Normal Colon | 0.1 | 0.0 | 0.1 | 0.3 | 0.0 |
| CC Well to Mod Diff (ODO3866) | 9.2 | 11.3 | 5.5 | 8.5 | 2.2 |
| CC Margin (ODO3866) | 0.2 | 0.0 | 0.0 | 0.3 | 0.0 |
| CC Gr.2 rectosigmoid (ODO3868) | 0.0 | 0.2 | 0.0 | 0.3 | 0.0 |
| CC Margin (ODO3868) | 0.0 | 0.0 | 0.0 | 0.0 | 0.0 |
| CC Mod Diff (ODO3920) | 0.2 | 0.1 | 0.0 | 0.5 | 0.0 |
| CC Margin (ODO3920) | 0.0 | 0.0 | 0.0 | 0.0 | 0.0 |
| CC Gr.2 ascend colon (ODO3921) | 0.0 | 0.0 | 0.1 | 0.1 | 0.0 |
| CC Margin (ODO3921) | 0.3 | 0.2 | 0.1 | 0.1 | 0.0 |
| CC from Partial Hepatectomy (ODO4309) Mets | 1.4 | 1.4 | 0.2 | 1.8 | 0.3 |
| Liver Margin (ODO4309) | 1.8 | 0.9 | 0.6 | 2.0 | 0.4 |
| Colon mets to lung (OD04451-01) | 0.2 | 0.2 | 0.3 | 0.4 | 0.2 |
| Lung Margin (OD04451-02) | 0.0 | 0.0 | 0.0 | 0.0 | 0.0 |
| Normal Prostate 6546-1 | 0.4 | 0.7 | 0.2 | 1.6 | 0.4 |
| Prostate Cancer (OD04410) | 0.0 | 0.0 | 0.0 | 0.0 | 0.0 |
| Prostate Margin (OD04410) | 0.0 | 0.0 | 0.0 | 0.4 | 0.0 |
| Prostate Cancer (OD04720-01) | 0.9 | 1.2 | 0.3 | 0.5 | 0.4 |
| Prostate Margin (OD04720-02) | 0.3 | 0.4 | 0.3 | 0.0 | 0.1 |
| Normal Lung | 0.0 | 0.0 | 0.1 | 0.0 | 0.0 |
| Lung Met to Muscle (ODO4286) | 0.0 | 0.3 | 0.0 | 0.0 | 0.2 |
| Muscle Margin (ODO4286) | 0.2 | 0.0 | 0.0 | 0.1 | 0.0 |
| Lung Malignant Cancer (OD03126) | 0.0 | 0.3 | 0.0 | 0.2 | 0.2 |
| Lung Margin (OD03126) | 0.0 | 0.1 | 0.0 | 0.0 | 0.0 |
| Lung Cancer (OD04404) | 0.0 | 0.0 | 0.0 | 0.0 | 0.1 |
| Lung Margin (OD04404) | 0.0 | 0.0 | 0.0 | 0.0 | 0.0 |
| Lung Cancer (OD04565) | 0.2 | 0.0 | 0.0 | 0.4 | 0.1 |
| Lung Margin (OD04565) | 0.0 | 0.0 | 0.0 | 0.0 | 0.0 |
| Lung Cancer (OD04237-01) | 0.0 | 0.2 | 0.0 | 0.5 | 0.0 |
| Lung Margin (OD04237-02) | 0.0 | 0.0 | 0.0 | 0.0 | 0.0 |
| Ocular Mel Met to Liver (ODO4310) | 0.0 | 0.0 | 0.0 | 0.0 | 0.0 |
| Liver Margin (ODO4310) | 1.0 | 1.1 | 0.0 | 1.0 | 0.5 |
| Melanoma Metastasis | 0.0 | 0.0 | 0.0 | 0.0 | 0.0 |
| Lung Margin (OD04321) | 0.0 | 0.0 | 0.0 | 0.0 | 0.0 |
| Normal Kidney | 0.3 | 1.1 | 0.1 | 0.7 | 0.3 |
| Kidney ca, Nuclear grade 2 (OD04338) | 0.0 | 0.0 | 0.1 | 0.1 | 0.0 |
| Kidney Margin (OD04338) | 0.2 | 0.7 | 0.2 | 0.2 | 0.1 |
| Kidney Ca Nuclear grade 1/2 (OD04339) | 0.1 | 0.1 | 0.3 | 0.4 | 0.0 |
| Kidney Margin (OD04339) | 0.6 | 0.3 | 0.7 | 1.4 | 0.3 |
| Kidney Ca, Clear cell type (OD04340) | 0.0 | 0.0 | 0.0 | 0.0 | 0.0 |
| Kidney Margin (OD04340) | 1.2 | 0.8 | 0.4 | 0.9 | 0.5 |
| Kidney Ca, Nuclear grade 3 (OD04348) | 0.0 | 0.0 | 0.0 | 0.6 | 0.0 |
| Kidney Margin (OD04348) | 0.4 | 0.4 | 0.4 | 0.0 | 0.3 |
| Kidney Cancer (OD04622-01) | 0.0 | 0.0 | 0.0 | 0.0 | 0.0 |
| Kidney Margin (OD04622-03) | 0.4 | 0.1 | 0.0 | 0.0 | 0.2 |
| Kidney Cancer (OD04450-01) | 0.3 | 0.2 | 0.1 | 0.0 | 0.0 |
| Kidney Margin (OD04450-03) | 0.0 | 0.2 | 0.0 | 0.3 | 0.2 |
| Kidney Cancer 8120607 | 0.0 | 0.0 | 0.0 | 0.0 | 0.0 |
| Kidney Margin 8120608 | 0.0 | 0.4 | 0.1 | 0.0 | 0.1 |
| Kidney Cancer 8120613 | 0.1 | 0.9 | 0.3 | 0.6 | 0.2 |
| Kidney Margin 8120614 | 0.3 | 0.3 | 0.0 | 0.0 | 0.0 |
| Kidney Cancer 9010320 | 0.0 | 0.1 | 0.0 | 0.0 | 0.0 |
| Kidney Margin 9010321 | 0.1 | 0.0 | 0.0 | 0.0 | 0.1 |
| Normal Uterus | 0.0 | 0.0 | 0.0 | 0.0 | 0.0 |
| Uterine Cancer 064011 | 0.0 | 0.0 | 0.0 | 0.0 | 0.0 |
| Normal Thyroid | 0.0 | 0.1 | 0.0 | 0.0 | 0.0 |
| Thyroid Cancer | 0.0 | 0.3 | 0.3 | 0.0 | 0.0 |
| Thyroid Cancer A302152 | 0.1 | 0.0 | 0.0 | 0.0 | 0.1 |
| Thyroid Margin A302153 | 0.0 | 0.0 | 0.0 | 0.0 | 0.0 |
| Normal Breast | 0.3 | 0.4 | 0.1 | 1.2 | 0.5 |
| Breast Cancer | 33.4 | 34.6 | 22.7 | 55.9 | 47.0 |
| Breast Cancer (OD04590-01) | 100.0 | 100.0 | 100.0 | 100.0 | 30.6 |
| Breast Cancer Mets (OD04590-03) | 63.3 | 72.7 | 73.7 | 88.9 | 100.0 |
| Breast Cancer Metastasis | 2.8 | 2.6 | 2.0 | 2.6 | 2.9 |
| Breast Cancer | 3.6 | 5.2 | 2.2 | 6.4 | 3.1 |
| Breast Cancer | 1.0 | 1.9 | 2.2 | 1.9 | 0.5 |
| Breast Cancer 9100266 | 3.5 | 3.3 | 1.1 | 7.1 | 4.1 |
| Breast Margin 9100265 | 0.0 | 0.3 | 0.1 | 0.2 | 0.3 |
| Breast Cancer A209073 | 2.3 | 1.6 | 0.7 | 2.0 | 1.7 |
| Breast Margin A209073 | 0.7 | 1.3 | 0.7 | 0.9 | 0.2 |
| Normal Liver | 0.1 | 0.5 | 0.4 | 2.2 | 0.9 |
| Liver Cancer | 0.3 | 0.3 | 0.4 | 0.3 | 0.1 |
| Liver Cancer 1025 | 0.4 | 0.7 | 0.5 | 1.3 | 0.5 |
| Liver Cancer 1026 | 0.7 | 0.1 | 0.3 | 1.1 | 0.2 |
| Liver Cancer 6004-T | 0.9 | 1.1 | 1.4 | 2.8 | 0.6 |
| Liver Tissue 6004-N | 0.0 | 0.0 | 0.1 | 0.0 | 0.0 |
| Liver Cancer 6005-T | 0.8 | 0.8 | 1.2 | 0.7 | 0.2 |
| Liver Tissue 6005-N | 0.1 | 0.0 | 0.2 | 0.5 | 0.0 |
| Normal Bladder | 0.0 | 0.0 | 0.1 | 0.4 | 0.1 |
| Bladder Cancer | 0.0 | 0.1 | 0.0 | 0.0 | 0.1 |
| Bladder Cancer | 0.1 | 0.4 | 0.3 | 0.0 | 0.1 |
| Bladder Cancer (OD04718-01) | 0.2 | 0.2 | 0.3 | 0.0 | 0.4 |
| Bladder Normal Adjacent (OD04718-03) | 0.0 | 0.0 | 0.0 | 0.0 | 0.0 |
| Normal Ovary | 0.0 | 0.0 | 0.0 | 0.0 | 0.0 |
| Ovarian Cancer | 0.0 | 0.0 | 0.0 | 0.2 | 0.0 |
| Ovarian Cancer (OD04768-07) | 0.1 | 0.0 | 0.1 | 0.0 | 0.0 |
| Ovary Margin (OD04768-08) | 0.0 | 0.0 | 0.0 | 0.0 | 0.0 |
| Normal Stomach | 0.0 | 0.1 | 0.0 | 0.2 | 0.0 |
| Gastric Cancer 9060358 | 0.0 | 0.0 | 0.0 | 0.0 | 0.0 |
| Stomach Margin 9060359 | 0.0 | 0.0 | 0.0 | 0.0 | 0.0 |
| Gastric Cancer 9060395 | 0.0 | 0.0 | 0.0 | 0.0 | 0.0 |
| Stomach Margin 9060394 | 0.0 | 0.0 | 0.0 | 0.0 | 0.0 |
| Gastric Cancer 9060397 | 1.5 | 0.7 | 0.6 | 0.3 | 0.2 |
| Stomach Margin 9060396 | 0.0 | 0.0 | 0.0 | 0.0 | 0.0 |
| Gastric Cancer 064005 | 0.1 | 0.3 | 0.0 | 0.2 | 0.2 |

TABLE AAN
Panel 3D
Column A - Rel. Exp. (%) Ag2899, Run 164633619
Column B - Rel. Exp. (%) Ag720, Run 164843791

| Tissue Name | A | B | Tissue Name | A | B |
|---|---|---|---|---|---|
| 94905 Daoy | 0.0 | 0.0 | 94954 Ca Ski Cervical | 0.0 | 3.2 |
| Medulloblastoma/Cerebellum | | | epidermoid carcinoma | | |
| | | | (metastasis | | |
| 94906 TE671 | 0.0 | 0.0 | 94955 ES-2 Ovarian clear cell | 0.0 | 0.0 |
| Medulloblastom/Cerebellum | | | carcinoma | | |
| 94907 D283 Med | 0.0 | 0.0 | 94957 Ramos Stimulated with | 0.0 | 0.0 |
| Medulloblastoma/Cerebellum | | | PMA/ionomycin 6 h | | |
| 94908 PFSK-1 Primitive | 0.0 | 1.4 | 94958 Ramos Stimulated with | 0.0 | 0.0 |
| Neuroectodermal/Cerebellum | | | PMA/ionomycin 14 h | | |
| 94909 XF-498 CNS | 0.0 | 0.0 | 94962 MEG-01 Chronic | 0.0 | 0.0 |
| | | | myelogenous leukemia | | |
| | | | (megokaryoblast) | | |
| 94910 SNB-78 CNS/glioma | 0.0 | 0.0 | 94963 Raji Burkitt's | 0.0 | 0.0 |
| | | | lymphoma | | |
| 94911 SF-268 CNS/glioblastoma | 0.0 | 0.0 | 94964 Daudi Burkitt's | 0.0 | 0.0 |
| | | | lymphoma | | |
| 94912 T98G Glioblastoma | 0.0 | 0.0 | 94965 U266 B-cell | 14.7 | 17.6 |
| | | | plasmacytoma/myeloma | | |
| 96776 SK-N-SH Neuroblastoma | 0.0 | 0.0 | 94968 CA46 Burkitt's | 0.0 | 0.0 |
| (metastasis) | | | lymphoma | | |
| 94913 SF-295 CNS/glioblastoma | 0.0 | 0.0 | 94970 RL non-Hodgkin's B- | 0.0 | 0.0 |
| | | | cell lymphoma | | |
| 94914 Cerebellum | 0.0 | 0.0 | 94972 JM1 pre-B-cell | 0.0 | 0.0 |
| | | | lymphoma/leukemia | | |
| 96777 Cerebellum | 1.2 | 0.0 | 94973 Jurkat T cell leukemia | 0.0 | 0.0 |
| 94916 NCI-H292 | 0.0 | 0.0 | 94974 TF-1 Erythroleukemia | 0.0 | 0.0 |
| Mucoepidermoid lung carcinoma | | | | | |
| 94917 DMS-114 Small cell lung | 0.0 | 0.0 | 94975 HUT 78 T-cell | 0.0 | 0.0 |
| cancer | | | lymphoma | | |
| 94918 DMS-79 Small cell lung | 100.0 | 100.0 | 94977 U937 Histiocytic | 0.0 | 0.0 |
| cancer/neuroendocrine | | | lymphoma | | |
| 94919 NCI-H146 Small cell lung | 0.0 | 0.0 | 94980 KU-812 Myelogenous | 0.0 | 0.0 |
| cancer/neuroendocrine | | | leukemia | | |
| 94920 NCI-H526 Small cell lung | 0.0 | 0.0 | 769-P- Clear cell renal | 0.0 | 0.0 |
| cancer/neuroendocrine | | | carcinoma | | |
| 94921 NCI-N417 Small cell lung | 0.0 | 0.0 | 94983 Caki-2 Clear cell renal | 0.8 | 1.9 |
| cancer/neuroendocrine | | | carcinoma | | |
| 94923 NCI-H82 Small cell lung | 0.0 | 0.0 | 94984 SW 839 Clear cell renal | 0.0 | 0.0 |
| cancer/neuroendocrine | | | carcinoma | | |
| 94924 NCI-H157 Squamous cell | 0.0 | 0.0 | 94986 G401 Wilms' tumor | 0.0 | 0.0 |
| lung cancer (metastasis) | | | | | |
| 94925 NCI-H1155 Large cell | 0.0 | 0.0 | 94987 Hs766T Pancreatic | 1.2 | 5.5 |
| lung cancer/neuroendocrine | | | carcinoma (LN metastasis) | | |
| 94926 NCI-H1299 Large cell | 0.0 | 0.0 | 94988 CAPAN-l Pancreatic | 0.0 | 0.0 |
| lung cancer/neuroendocrine | | | adenocarcinoma (liver | | |
| | | | metastasis) | | |
| 94927 NCI-H727 Lung carcinoid | 0.0 | 0.0 | 94989 SU86.86 Pancreatic | 0.0 | 0.0 |
| | | | carcinoma (liver metastasis) | | |
| 94928 NCI-UMC-11 Lung | 0.0 | 4.9 | 94990 BxPC-3 Pancreatic | 0.0 | 2.9 |
| carcinoid | | | adenocarcinoma | | |
| 94929 LX-1 Small cell lung | 3.2 | 3.7 | 94991 HPAC Pancreatic | 1.2 | 5.1 |
| cancer | | | adenocarcinoma | | |
| 94930 Colo-205 Colon cancer | 7.5 | 29.3 | 94992 MIA PaCa-2 Pancreatic | 0.0 | 0.0 |
| | | | carcinoma | | |
| 94931 KM12 Colon cancer | 0.2 | 1.6 | 94993 CFPAC-1 Pancreatic | 29.1 | 47.6 |
| | | | ductal adenocarcinoma | | |
| 94932 KM20L2 Colon cancer | 1.2 | 0.0 | 94994 PANC-1 Pancreatic | 0.0 | 0.0 |
| | | | epithelioid ductal carcinoma | | |
| 94933 NCI-H716 Colon cancer | 0.0 | 0.0 | 94996 T24 Bladder carcinma | 0.0 | 0.0 |
| | | | (transitional cell | | |
| 94935 SW-48 Colon | 0.0 | 0.0 | 5637-Bladder carcinoma | 0.0 | 0.0 |
| adenocarcinoma | | | | | |
| 94936 SW1116 Colon | 0.0 | 0.0 | 94998 HT-1197 Bladder | 5.1 | 7.6 |
| adenocarcinoma | | | carcinoma | | |
| 94937 LS 174T Colon | 1.1 | 0.0 | 94999 UM-UC-3 Bladder | 0.0 | 0.0 |
| adenocarcinoma | | | carcinma (transitional cell) | | |
| 94938 SW-948 Colon | 0.0 | 0.0 | 95000 A204 | 0.0 | 0.0 |
| adenocarcinoma | | | Rhabdomyosarcoma | | |
| 94939 SW-480 Colon | 0.0 | 0.0 | 95001 HT-1080 Fibrosarcoma | 0.0 | 0.0 |
| adenocarcinoma | | | | | |
| 94940 NCI-SNU-5 Gastric | 0.0 | 1.1 | 95002 MG-63 Osteosarcoma | 0.0 | 0.0 |
| carcinoma | | | (bone) | | |
| KATO III-Gastric carcinoma | 0.0 | 1.9 | 95003 SK-LMS-1 | 0.0 | 0.0 |
| | | | Leiomyosarcoma (vulva) | | |
| 94943 NCI-SNU-16 Gastric | 2.2 | 0.0 | 95004 SJRH30 | 0.0 | 0.0 |
| carcinoma | | | Rhabdomyosarcoma (met to | | |
| | | | bone marrow) | | |
| 94944 NCI-SNU-1 Gastric | 0.0 | 0.0 | 95005 A431 Epidermoid | 1.8 | 1.6 |
| carcinoma | | | carcinoma | | |
| 94946 RF-1 Gastric | 0.0 | 0.0 | 95007 WM266-4 Melanoma | 0.0 | 0.0 |
| adenocarcinoma | | | | | |
| 94947 RF-48 Gastric | 0.0 | 0.0 | DU 145-Prostate carcinoma | 0.0 | 0.0 |
| adenocarcinoma | | | (brain metastasis) | | |
| 96778 MKN-45 Gastric | 0.0 | 0.0 | 95012 MDA-MB-468 Breast | 1.1 | 0.0 |
| carcinoma | | | adenocarcinoma | | |
| 94949 NCI-N87 Gastric | 0.0 | 3.8 | SCC-4-Squamous cell | 0.0 | 0.0 |
| carcinoma | | | carcinoma of tongue | | |
| 94951 OVCAR-5 Ovarian | 29.7 | 62.0 | SCC-9-Squamous cell | 0.0 | 0.0 |
| carcinoma | | | carcinoma of tongue | | |
| 94952 RL95-2 Uterine carcinoma | 19.3 | 21.8 | SCC-15-Squamous cell | 0.0 | 0.0 |
| | | | carcinoma of tongue | | |
| 94953 HelaS3 Cervical | 0.0 | 0.0 | 95017 CAL 27 Squamous cell | 0.0 | 0.0 |
| adenocarcinoma | | | carcinoma of tongue | | |

TABLE AAO
Panel 4D
Column A - Rel. Exp.(%) Ag1590, Run 152061102
Column B - Rel. Exp.(%) Ag1590, Run 155330411
Column C - Rel. Exp.(%) Ag1918, Run 147288180
Column D - Rel. Exp.(%) Ag2899, Run 159633215

| Tissue Name | A | B | C | D |
|---|---|---|---|---|
| Secondary Th1 act | 0.0 | 0.0 | 0.0 | 0.0 |
| Secondary Th2 act | 0.0 | 0.0 | 0.0 | 4.3 |
| Secondary Tr1 act | 0.0 | 0.0 | 0.0 | 6.6 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.0 | 0.0 | 0.0 |
| Primary Tr1 act | 0.0 | 0.0 | 0.0 | 0.0 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 lymphocyte act | 0.0 | 0.0 | 0.0 | 0.0 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 0.0 | 0.0 | 0.0 |
| Secondary CD8 lymphocyte act | 0.0 | 0.0 | 0.0 | 0.0 |
| CD4 lymphocyte none | 0.0 | 0.0 | 0.0 | 0.0 |
| 2ry Th1/Th2/Tr1 anti-CD95 CH11 | 0.0 | 0.0 | 0.0 | 0.0 |
| LAK cells rest | 0.0 | 0.0 | 0.0 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.0 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 6.3 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 0.0 | 0.0 | 0.0 | 0.0 |
| B lymphocytes CD40L and IL-4 | 0.0 | 0.0 | 0.0 | 0.0 |
| EOL-1 dbcAMP | 0.0 | 0.0 | 0.0 | 0.0 |
| EOL-1 dbcAMP PMA/ionomycin | 0.0 | 0.0 | 0.0 | 0.0 |
| Dendritic cells none | 0.0 | 0.0 | 0.0 | 0.0 |
| Dendritic cells LPS | 0.0 | 0.0 | 0.0 | 0.0 |
| Dendritic cells anti-CD40 | 0.0 | 0.0 | 0.0 | 0.0 |
| Monocytes rest | 0.0 | 0.0 | 0.0 | 12.9 |
| Monocytes LPS | 0.0 | 0.0 | 0.0 | 0.0 |
| Macrophages rest | 0.0 | 0.0 | 0.0 | 0.0 |
| Macrophages LPS | 0.0 | 0.0 | 0.0 | 0.0 |
| HUVEC none | 0.0 | 0.0 | 0.0 | 0.0 |
| HUVEC starved | 0.0 | 0.0 | 0.0 | 0.0 |
| HUVEC IL-1beta | 0.0 | 0.0 | 0.0 | 0.0 |
| HUVEC IFN gamma | 0.0 | 0.0 | 0.0 | 0.0 |
| HUVEC TNF alpha + IFN gamma | 0.0 | 0.0 | 0.0 | 0.0 |
| HUVEC TNF alpha + IL4 | 0.0 | 0.0 | 0.0 | 0.0 |
| HUVEC IL-11 | 0.0 | 0.0 | 0.0 | 0.0 |
| Lung Microvascular EC none | 0.0 | 0.0 | 0.0 | 0.0 |
| Lung Microvascular EC TNF alpha + | 0.0 | 0.0 | 0.0 | 0.0 |
| IL-1beta | | | | |
| Microvascular Dermal EC none | 0.0 | 0.0 | 0.0 | 0.0 |
| Microsvasular Dermal EC TNF alpha + | 0.0 | 0.0 | 0.0 | 0.0 |
| IL-1beta | | | | |
| Bronchial epithelium TNF alpha + IL1beta | 13.9 | 0.0 | 30.1 | 3.4 |
| Small airway epithelium none | 0.0 | 0.0 | 0.0 | 0.0 |
| Small airway epithelium TNF alpha + | 0.0 | 0.0 | 0.0 | 0.0 |
| IL-1beta | | | | |
| Coronery artery SMC rest | 0.0 | 0.0 | 0.0 | 0.0 |
| Coronery artery SMC TNF alpha + IL-1beta | 0.0 | 0.0 | 0.0 | 0.0 |
| Astrocytes rest | 0.0 | 0.0 | 0.0 | 0.0 |
| Astrocytes TNF alpha + IL-1beta | 0.0 | 0.0 | 0.0 | 0.0 |
| KU-812 (Basophil) rest | 0.0 | 0.0 | 0.0 | 0.0 |
| KU-812 (Basophil) PMA/ionomycin | 10.8 | 0.0 | 0.0 | 5.4 |
| CCD1106 (Keratinocytes) none | 0.0 | 0.0 | 0.0 | 0.0 |
| CCD1106 (Keratinocytes) TNF alpha + | 0.0 | 0.0 | 0.0 | 0.0 |
| IL-1beta | | | | |
| Liver cirrhosis | 22.7 | 80.1 | 63.7 | 100.0 |
| Lupus kidney | 0.0 | 7.4 | 0.0 | 0.0 |
| NCI-H292 none | 60.3 | 92.7 | 36.3 | 28.9 |
| NCI-H292 IL-4 | 55.9 | 6.5 | 28.7 | 14.3 |
| NCI-H292 IL-9 | 100.0 | 41.8 | 40.1 | 29.7 |
| NCI-H292 IL-13 | 35.8 | 6.7 | 9.4 | 23.7 |
| NCI-H292 IFN gamma | 0.0 | 23.8 | 1.2 | 0.0 |
| HPAEC none | 0.0 | 0.0 | 0.0 | 0.0 |
| HPAEC TNF alpha + IL-1beta | 0.0 | 0.0 | 0.0 | 0.0 |
| Lung fibroblast none | 0.0 | 0.0 | 0.0 | 0.0 |
| Lung fibroblast TNF alpha + IL-1beta | 0.0 | 0.0 | 0.0 | 0.0 |
| Lung fibroblast IL-4 | 0.0 | 0.0 | 0.0 | 0.0 |
| Lung fibroblast IL-9 | 0.0 | 0.0 | 0.0 | 0.0 |
| Lung fibroblast IL-13 | 0.0 | 0.0 | 0.0 | 0.0 |
| Lung fibroblast IFN gamma | 0.0 | 0.0 | 0.0 | 0.0 |
| Dermal fibroblast CCD1070 rest | 0.0 | 0.0 | 0.0 | 0.0 |
| Dermal fibroblast CCD1070 TNF alpha | 0.0 | 0.0 | 0.0 | 0.0 |
| Dermal fibroblast CCD1070 IL-1beta | 0.0 | 0.0 | 0.0 | 0.0 |
| Dermal fibroblast IFN gamma | 0.0 | 0.0 | 0.0 | 0.0 |
| Dermal fibroblast IL-4 | 0.0 | 0.0 | 0.0 | 0.0 |
| IBD Colitis 2 | 0.0 | 0.0 | 0.0 | 0.0 |
| IBD Crohn's | 13.8 | 0.0 | 0.0 | 0.0 |
| Colon | 79.6 | 100.0 | 100.0 | 74.7 |
| Lung | 77.9 | 66.0 | 53.6 | 85.3 |
| Thymus | 62.0 | 21.6 | 0.7 | 23.0 |
| Kidney | 0.0 | 0.0 | 0.0 | 0.0 |

Ardais Breast1.0 Summary: Ag720 Expression of this gene was highest in a breast cancer sample (CT=27.1). Significant CG53989-03 gene expression was detected in 45/64 breast cancer samples but only 1/7 normal breast samples. Gene or protein expression levels are useful for the detection of breast cancer. Therapeutic modulation of this gene, encoded protein and/or use of antibodies or small molecule drugs targeting this gene or gene product is useful in the treatment of breast cancer.

This gene encodes a protein with homology to mastocytoma protease precursor. Mast cell tryptase is a secretory granule associated serine protease with trypsin-like specificity. It is released extracellularly during mast cell degranulation. Mast cells (MC) have been associated with diverse human cancers. The primary function of these cells is to store and release a number of biologically active mediators, including the serine proteases tryptase and chymase. These proteases have been closely related with angiogenesis and tumor invasion, two critical steps during tumor progression. Malignant breast tumors have two to three times more tryptase-containing than chymase-containing mast cells, with the number of mast cells with trptase activity being significantly higher (p<0.02) than in benign lesions. In malignant lesions, tryptase-containing mast cells were concentrated at the tumor edge, i.e. the invasion zone (Kankkunen J P, Harvima I T, Naukkarinen A. Quantitative analysis of tryptase and chymase containing mast cells in benign and malignant breast lesions. Int J Cancer. 1997 Jul. 29; 72(3): 385-8). Therefore, the protease encoded by this gene plays a role in tumor invasion and metastasis.

General_screening_panel_v1.6 Summary: Ag6974 Highest expression of this gene was detected in a ovarian cancer OVCAR-5 cell line (CT=28). This gene showed preferential expression in colon cancer tissue and a number of cancer cell lines derived from pancreatic, colon, gastric, lung, breast and ovarian cancers. Expression of this gene is useful as diagnostic marker to detect these cancers and also, modulation of this gene, encoded protein and/or use of antibodies or small molecule drug targeting this gene or gene product is useful in the treatment of these cancers.

Panel 1.3D Summary: Ag1590/2899 The expression of the CG56242-01 gene was assessed in four independent runs using two different probe/primer sets. All of the runs show excellent concordance. The expression of this gene was highest in a sample derived from an ovarian cancer cell line (OVCAR-5) (CTs=31-32). There was significant expression associated with a colon cancer cell line, a gastric cancer cell line and pituitary tissue. Therapeutic modulation of this gene, encoded protein and/or use of small molecule or antibodies targeting this gene or gene product is useful in the treatment of ovarian cancer, gastric cancer or colon cancers.

Panel 2D Summary: Ag720/1590/2899 The expression of this gene was highest and exclusive to breast cancer samples (CTs=26-28). Thus, the expression of this gene is useful as marker for breast cancer. Therapeutic modulation of this gene, encoded protein and/or use of small molecule or antibodies targeting this gene or gene product is useful in the treatment of breast cancer.

Panel 3D Summary: Ag720/2899 The expression of this gene was highest in a sample derived from a lung cancer cell line (DMS-79)(CTs=29-31). There was low but significant expression associated with samples derived from an ovarian cancer cell line, a uterine cancer cell line and a pancreatic cancer cell line. The expression of this gene or expressed protein is useful in the detection of lung cancer. Therapeutic modulation of this gene, expressed protein and/or use of antibodies or small molecule drugs targeting the gene or gene product are useful in the treatment of lung cancer, ovarian cancer, pancreatic cancer or uterine cancer.

Panel 4D Summary: Ag1590/Ag1918/Ag2899 This gene, a tryptase homolog, was expressed at significant levels in IL-9-activated NCI-H292 cells, pulmonary mucoepidermoid cells. Colon, lung, and thymus tissues also showed low levels of expression of this gene. The expression in lung and in the activated NCI-H292 cell line, often used as a model for airway epithelium was consistent with published reports of tryptase in the lung (Walls A F, Bennett A R, Godfrey R C, Holgate S T, Church M K. Mast cell tryptase and histamine concentrations in bronchoalveolar lavage fluid from patients with interstitial lung disease. Clin Sci (Lond) 1991 August; 81(2):183-8). In addition, tryptase has been shown to be up-regulated in lungs affected by disease and specifically in COPD (Grashoff W F, Sont J K, Sterk P J, Hiemstra P S, de Boer W I, Stolk J, Han J, van Krieken J M. Chronic obstructive pulmonary disease: role of bronchiolar mast cells and macrophages. Am J Pathol 1997 December; 151(6):1785-90). Tryptase has also been implicated in the recruitment of granulocytes and epithelial repair (Cairns J A, Walls A F. Mast cell tryptase is a mitogen for epithelial cells. Stimulation of IL-8 production and intercellular adhesion molecule-1 expression. J Immunol 1996 Jan. 1; 156(1):275-83). Based on these observations, therapeutic modulation of this gene, encoded protein and/use of small molecule or antibodies targeting this gene is useful in the reduction or elimination of symptoms in patients with lung diseases including asthma, allergy, or chronic obstructive pulmonary disease.

AB. CG54212-02: GPCR.

Expression of gene CG54212-02 was assessed using the primer-probe set Ag431, described in Table ABA. Results of the RTQ-PCR runs are shown in Tables ABB, ABC and ABD.

TABLE ABA
Probe Name Ag431

| | | | Start | SEQ ID |
|---|---|---|---|---|
| Primers | Sequences | Length | Position | No |
| Forward | 5′-agtcacttcacctgcaagatcct-3′ | 23 | 533 | 1271 |
| Probe | TET-5′-ccgcatgccagcttcagcactg-3′- | 22 | 558 | 1272 |
| | TAMRA | | | |
| Reverse | 5′-cttcgctgaccgacgtgtt-3′ | 19 | 581 | 1273 |

TABLE ABB
Panel 1
Column A - Rel. Exp.(%) Ag431, Run 98747695

| Tissue Name | A | Tissue Name | A |
|---|---|---|---|
| Endothelial cells | 0.0 | Renal ca. 786-0 | 0.0 |
| Endothelial cells | 0.0 | Renal ca. A498 | 1.5 |
| (treated) | | | |
| Pancreas | 0.0 | Renal ca. RXF 393 | 0.0 |
| Pancreatic ca. | 0.0 | Renal ca. ACHN | 0.0 |
| CAPAN 2 | | | |
| Adrenal gland | 2.0 | Renal ca. UO-31 | 0.1 |
| Thyroid | 0.0 | Renal ca. TK-10 | 0.2 |
| Salivary gland | 0.1 | Liver | 0.1 |
| Pituitary gland | 0.4 | Liver (fetal) | 0.2 |
| Brain (fetal) | 27.5 | Liver ca. (hepatoblast) HepG2 | 0.0 |
| Brain (whole) | 21.9 | Lung | 1.3 |
| Brain (amygdala) | 36.1 | Lung (fetal) | 0.6 |
| Brain (cerebellum) | 28.5 | Lung ca. (small cell) LX-1 | 9.9 |
| Brain (hippocampus) | 36.1 | Lung ca. (small cell) NCI-H69 | 18.4 |
| Brain (substantia | 17.1 | Lung ca. (s. cell var.) SHP-77 | 0.0 |
| nigra) | | | |
| Brain (thalamus) | 36.1 | Lung ca. (large cell) NCI-H460 | 5.3 |
| Brain (hypothalamus) | 13.5 | Lung ca. (non-sm. cell) A549 | 8.4 |
| Spinal cord | 6.7 | Lung ca. (non-s. cell) NCI-H23 | 0.4 |
| glio/astro U87-MG | 0.0 | Lung ca. (non-s. cell) HOP-62 | 0.1 |
| glio/astro U-118-MG | 0.0 | Lung ca. (non-s. cl) NCI-H522 | 5.8 |
| astrocytoma SW1783 | 0.0 | Lung ca. (squam.) SW 900 | 13.5 |
| neuro*; met SK-N-AS | 0.1 | Lung ca. (squam.) NCI-H596 | 6.6 |
| astrocytoma SF-539 | 0.1 | Mammary gland | 25.2 |
| astrocytoma SNB-75 | 0.1 | Breast ca.* (pl.ef) MCF-7 | 0.9 |
| glioma SNB-19 | 2.6 | Breast ca.* (pl.ef) MDA-MB-231 | 0.0 |
| glioma U251 | 8.2 | Breast ca.* (pl.ef) T47D | 4.3 |
| glioma SF-295 | 2.6 | Breast ca. BT-549 | 0.3 |
| Heart | 4.0 | Breast ca. MDA-N | 0.2 |
| Skeletal muscle | 32.1 | Ovary | 0.4 |
| Bone marrow | 0.0 | Ovarian ca. OVCAR-3 | 1.4 |
| Thymus | 2.5 | Ovarian ca. OVCAR-4 | 0.1 |
| Spleen | 0.1 | Ovarian ca. OVCAR-5 | 17.9 |
| Lymph node | 0.0 | Ovarian ca. OVCAR-8 | 17.6 |
| Colon (ascending) | 16.0 | Ovarian ca. IGROV-1 | 8.1 |
| Stomach | 0.6 | Ovarian ca. (ascites) SK-OV-3 | 0.9 |
| Small intestine | 0.5 | Uterus | 1.0 |
| Colon ca. SW480 | 0.0 | Placenta | 0.5 |
| Colon ca.* SW620 | 0.0 | Prostate | 7.2 |
| (SW480 met) | | | |
| Colon ca. HT29 | 4.5 | Prostate ca.* (bone met) PC-3 | 2.0 |
| Colon ca. HCT-116 | 0.0 | Testis | 20.9 |
| Colon ca. CaCo-2 | 0.0 | Melanoma Hs688(A).T | 0.0 |
| Colon ca. HCT-15 | 10.5 | Melanoma* (met) Hs688(B).T | 1.6 |
| Colon ca. HCC-2998 | 9.6 | Melanoma UACC-62 | 1.9 |
| Gastric ca. (liver met) | 8.5 | Melanoma M14 | 0.1 |
| NCI-N87 | | | |
| Bladder | 5.3 | Melanoma LOX IMVI | 0.0 |
| Trachea | 0.0 | Melanoma* (met) SK-MEL-5 | 0.0 |
| Kidney | 1.0 | Melanoma SK-MEL-28 | 100.0 |
| Kidney (fetal) | 3.2 | | |

TABLE ABC
Panel 2D
Column A - Rel. Exp.(%) Ag431, Run 153681087

| Tissue Name | A | Tissue Name | A |
|---|---|---|---|
| Normal Colon | 2.0 | Kidney Margin 8120608 | 0.0 |
| CC Well to Mod Diff (ODO3866) | 1.4 | Kidney Cancer 8120613 | 0.0 |
| CC Margin (ODO3866) | 0.0 | Kidney Margin 8120614 | 0.0 |
| CC Gr.2 rectosigmoid (ODO3868) | 1.6 | Kidney Cancer 9010320 | 0.0 |
| CC Margin (ODO3868) | 2.0 | Kidney Margin 9010321 | 20.7 |
| CC Mod Diff (ODO3920) | 0.8 | Normal Uterus | 1.8 |
| CC Margin (ODO3920) | 1.1 | Uterine Cancer 064011 | 4.9 |
| CC Gr.2 ascend colon (ODO3921) | 1.9 | Normal Thyroid | 0.0 |
| CC Margin (ODO3921) | 2.1 | Thyroid Cancer | 0.0 |
| CC from Partial Hepatectomy | 0.0 | Thyroid Cancer A302152 | 0.0 |
| (ODO4309) Mets | | | |
| Liver Margin (ODO4309) | 1.2 | Thyroid Margin A302153 | 1.9 |
| Colon mets to lung (OD04451-01) | 9.2 | Normal Breast | 0.9 |
| Lung Margin (OD04451-02) | 0.7 | Breast Cancer | 29.5 |
| Normal Prostate 6546-1 | 3.6 | Breast Cancer (OD04590-01) | 2.8 |
| Prostate Cancer (OD04410) | 3.1 | Breast Cancer Mets (OD04590-03) | 0.0 |
| Prostate Margin (OD04410) | 7.0 | Breast Cancer Metastasis | 14.6 |
| Prostate Cancer (OD04720-01) | 6.9 | Breast Cancer | 9.4 |
| Prostate Margin (OD04720-02) | 7.9 | Breast Cancer | 100.0 |
| Normal Lung | 0.6 | Breast Cancer 9100266 | 4.0 |
| Lung Met to Muscle (ODO4286) | 0.0 | Breast Margin 9100265 | 1.3 |
| Muscle Margin (ODO4286) | 0.6 | Breast Cancer A209073 | 1.4 |
| Lung Malignant Cancer (OD03126) | 2.0 | Breast Margin A209073 | 2.7 |
| Lung Margin (OD03126) | 4.2 | Normal Liver | 0.0 |
| Lung Cancer (OD04404) | 0.0 | Liver Cancer | 0.7 |
| Lung Margin (OD04404) | 0.9 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 2.2 |
| Lung Cancer (OD04237-01) | 0.5 | Liver Tissue 6004-N | 2.4 |
| Lung Margin (OD04237-02) | 0.0 | Liver Cancer 6005-T | 0.0 |
| Ocular Mel Met to Liver (OD04310) | 0.0 | Liver Tissue 6005-N | 0.0 |
| Liver Margin (OD04310) | 0.0 | Normal Bladder | 3.6 |
| Melanoma Metastasis | 0.0 | Bladder Cancer | 2.8 |
| Lung Margin (OD04321) | 2.5 | Bladder Cancer | 2.8 |
| Normal Kidney | 2.5 | Bladder Cancer (OD04718-01) | 0.8 |
| Kidney Ca, Nuclear grade 2 (OD04338) | 0.9 | Bladder Normal Adjacent | 3.2 |
| | | (OD04718-03) | |
| Kidney Margin (OD04338) | 0.0 | Normal Ovary | 0.0 |
| Kidney Ca Nuclear grade ½ (OD04339) | 0.0 | Ovarian Cancer | 2.0 |
| Kidney Margin (OD04339) | 3.1 | Ovarian Cancer (OD04768-07) | 0.0 |
| Kidney Ca, Clear cell type (OD04340) | 0.0 | Ovary Margin (OD04768-08) | 0.0 |
| Kidney Margin (OD04340) | 0.0 | Normal Stomach | 4.8 |
| Kidney Ca, Nuclear grade 3 (OD04348) | 0.0 | Gastric Cancer 9060358 | 0.0 |
| Kidney Margin (OD04348) | 2.7 | Stomach Margin 9060359 | 0.0 |
| Kidney Cancer (OD04622-01) | 1.1 | Gastric Cancer 9060395 | 2.9 |
| Kidney Margin (OD04622-03) | 0.0 | Stomach Margin 9060394 | 0.6 |
| Kidney Cancer (OD04450-01) | 0.0 | Gastric Cancer 9060397 | 0.6 |
| Kidney Margin (OD04450-03) | 5.0 | Stomach Margin 9060396 | 0.0 |
| Kidney Cancer 8120607 | 0.0 | Gastric Cancer 064005 | 0.6 |

TABLE ABD
Panel 4D
Column A - Rel. Exp.(%) Ag431, Run 153681094

| Tissue Name | A | Tissue Name | A |
|---|---|---|---|
| Secondary Th1 act | 0.0 | HUVEC IL-1beta | 0.0 |
| Secondary Th2 act | 0.1 | HUVEC IFN gamma | 0.1 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.1 |
| Secondary Tr1 rest | 0.0 | Lung Microvascular EC none | 0.0 |
| Primary Th1 act | 0.0 | Lung Microvascular EC TNF alpha + IL- | 0.0 |
| | | 1beta | |
| Primary Th2 act | 0.2 | Microvascular Dermal EC none | 0.0 |
| Primary Tr1 act | 0.0 | Microsvasular Dermal EC TNF alpha + IL- | 0.0 |
| | | 1beta | |
| Primary Th1 rest | 0.0 | Bronchial epithelium TNF alpha + IL1beta | 0.3 |
| Primary Th2 rest | 0.0 | Small airway epithelium none | 0.0 |
| Primary Tr1 rest | 0.1 | Small airway epithelium TNF alpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 1.8 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNF alpha + IL- | 0.0 |
| | | 1beta | |
| LAK cells IL-2 | 0.0 | Liver cirrhosis | 0.5 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.4 |
| LAK cells PMA/ionomycin | 0.1 | NCI-H292 IL-9 | 0.2 |
| NK Cells IL-2 rest | 0.0 | NCI-H292 IL-13 | 0.0 |
| Two Way MLR 3 day | 5.1 | NCI-H292 IFN gamma | 100.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL-1beta | 0.0 |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.2 |
| B lymphocytes PWM | 0.1 | Lung fibroblast IFN gamma | 0.0 |
| B lymphocytes CD40L and IL-4 | 0.4 | Dermal fibroblast CCD1070 rest | 0.1 |
| EOL-1 dbcAMP | 0.0 | Dermal fibroblast CCD1070 TNF alpha | 0.2 |
| EOL-1 dbcAMP | 0.0 | Dermal fibroblast CCD1070 IL-1beta | 0.0 |
| PMA/ionomycin | | | |
| Dendritic cells none | 0.2 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.1 |
| Monocytes rest | 0.0 | IBD Crohn's | 0.0 |
| Monocytes LPS | 0.0 | Colon | 0.2 |
| Macrophages rest | 0.0 | Lung | 0.2 |
| Macrophages LPS | 0.0 | Thymus | 0.1 |
| HUVEC none | 0.0 | Kidney | 0.2 |
| HUVEC starved | 0.0 | | |

Panel 1 Summary: Ag431 Expression of this gene was highest in a melanoma cell line (CT=27.1). Significant expression was also detected in ovarian, lung, and colon cancer cell lines. Modulation of this gene, encoded protein and/or use of small molecule drugs or antibodies targeting this gene or gene product is useful in the treatment of melanoma and lung, colon or ovarian cancers.

Among tissues with metabolic function, this gene was expressed in the pituitary and adrenal glands, the hypothalamus, heart and skeletal muscle. Modulation of this gene, encoded protein and/or use of antibodies or small molecule drug targeting this gene or gene product is useful in the treatment of metabolic/endocrine diseases such as diabetes and obesity.

This gene was also expressed at moderate levels in all the regions of the central nervous system examined including the fetal brain, cerebellum, amygdala, hippocampus, substantia nigra, thalamus, hypothalamus, and spinal cord. This gene codes for a GPCR. Neurotransmitter receptors belong to GPCR family of proteins. Thus, this protein may represent a novel neurotransmitter receptor. Neurotransmitter receptors that are GPCRs include the dopamine receptor family, the serotonin receptor family, the GABA receptor, and muscarinic acetylcholine receptors. The selected targeting of dopamine and serotonin receptors has proven to be effective in the treatement of psychiatric illnesses such as schizophrenia, bipolar disorder and depression. Furthermore, the cerebral cortex and hippocampus regions of the brain are known to play critical roles in Alzheimer's disease, seizure disorders, and in the normal process of memory formation. Therefore, modulation of this gene and/or encoded protein is useful in the treatment of any of these diseases.

Panel 2D Summary: Ag431 Expression of this gene was highest in breast cancer (CT=32.3) and was not detected at significant levels in normal breast tissue. There was significant expression in number of breast cancer samples. Modulation of this gene and encoded protein is useful in the treatment of breast cancer.

Panel 4D Summary: Ag431 This gene was expressed in IFN-gamma-stimulated mucoepidermoid (mucus-producing) NCI-H292 cells, but not in resting NCI-H292 cells, or in IL-4-, IL-9-, or IL-13-stimulated NCI-H292 cells. The gene was also expressed at low but significant levels at the three-day time point in a two-way mixed lymphocyte reaction with cells from normal human donors. Thus, modulation of this gene, encoded protein and/or use of antibody or small molecule drug targeting this gene or gene product is useful int he treatment of inflammatory diseases such as colitis, chronic obstructive pulmonary disease, asthma, allergy and emphysema.

AC. CG5423601: Cysteinyl Leukotriene Receptor 2.

Expression of gene CG54236-01 was assessed using the primer-probe set Ag2695, described in Table ACA. Results of the RTQ-PCR runs are shown in Tables ACB, ACC, ACD, ACE and ACF.

TABLE ACA
Probe Name Ag2695

| | | | Start | SEQ ID |
|---|---|---|---|---|
| Primers | Sequences | Length | Position | No |
| Forward | 5′-gggaaatgggttgtccatatat-3′ | 22 | 266 | 1274 |
| Probe | TET-5′-tcctgcagccttataagaagtccaca-3′- | 26 | 292 | 1275 |
| | TAMRA | | | |
| Reverse | 5′-atctgaaatggccagatttagc-3′ | 22 | 335 | 1276 |

TABLE ACB
AI_comprehensive panel_v1.0
Column A - Rel. Exp. (%) Ag2695, Run 249247284
Column B - Rel. Exp. (%) Ag2695, Run 249259794

| Tissue Name | A | B | Tissue Name | A | B |
|---|---|---|---|---|---|
| 110967 COPD-F | 7.1 | 2.5 | 112427 Match Control Psoriasis-F | 54.0 | 53.2 |
| 110980 COPD-F | 6.2 | 0.0 | 112418 Psoriasis-M | 0.0 | 6.4 |
| 110968 COPD-M | 6.9 | 7.9 | 112723 Match Control Psoriasis- | 0.0 | 0.0 |
| | | | M | | |
| 110977 COPD-M | 12.3 | 15.2 | 112419 Psoriasis-M | 12.5 | 12.5 |
| 110989 Emphysema-F | 45.1 | 28.5 | 112424 Match Control Psoriasis- | 19.5 | 11.0 |
| | | | M | | |
| 110992 Emphysema-F | 15.4 | 7.6 | 112420 Psoriasis-M | 85.9 | 75.3 |
| 110993 Emphysema-F | 11.9 | 4.8 | 112425 Match Control Psoriasis- | 48.0 | 58.2 |
| | | | M | | |
| 110994 Emphysema-F | 2.5 | 0.0 | 104689 (MF) OA Bone-Backus | 39.0 | 59.9 |
| 110995 Emphysema-F | 12.9 | 38.4 | 104690 (MF) Adj “Normal” Bone- | 21.8 | 27.7 |
| | | | Backus | | |
| 110996 Emphysema-F | 12.2 | 0.0 | 104691 (ME) OA Synovium- | 77.4 | 68.3 |
| | | | Backus | | |
| 110997 Asthma-M | 4.2 | 3.2 | 104692 (BA) OA Cartilage- | 0.0 | 0.0 |
| | | | Backus | | |
| 111001 Asthma-F | 46.3 | 25.2 | 104694 (BA) OA Bone-Backus | 48.3 | 47.0 |
| 111002 Asthma-F | 52.5 | 27.5 | 104695 (BA) Adj “Normal” Bone- | 24.8 | 21.5 |
| | | | Backus | | |
| 111003 Atopic Asthma-F | 58.2 | 46.0 | 104696 (BA) OA Synovium- | 81.8 | 49.7 |
| | | | Backus | | |
| 111004 Atopic Asthma-F | 42.3 | 53.2 | 104700 (SS) OA Bone-Backus | 26.8 | 15.2 |
| 111005 Atopic Asthma-F | 26.4 | 26.8 | 104701 (SS) Adj “Normal” Bone- | 13.5 | 11.4 |
| | | | Backus | | |
| 111006 Atopic Asthma-F | 7.1 | 13.7 | 104702 (SS) OA Synovium- | 62.0 | 49.0 |
| | | | Backus | | |
| 111417 Allergy-M | 20.0 | 18.8 | 117093 OA Cartilage Rep7 | 89.5 | 59.0 |
| 112347 Allergy-M | 0.0 | 0.0 | 112672 OA Bone5 | 16.7 | 38.2 |
| 112349 Normal Lung-F | 0.0 | 0.0 | 112673 OA Synovium5 | 15.7 | 11.3 |
| 112357 Normal Lung-F | 34.4 | 28.9 | 112674 OA Synovial Fluid cells5 | 0.0 | 12.9 |
| 112354 Normal Lung-M | 98.6 | 55.5 | 117100 OA Cartilage Rep14 | 7.6 | 6.1 |
| 112374 Crohns-F | 5.6 | 7.2 | 112756 OA Bone9 | 22.5 | 7.9 |
| 112389 Match Control | 11.0 | 5.2 | 112757 OA Synovium9 | 1.7 | 0.0 |
| Crohns-F | | | | | |
| 112375 Crohns-F | 7.8 | 2.5 | 112758 OA Synovial Fluid Cells9 | 6.7 | 20.4 |
| 112732 Match Control | 43.5 | 18.6 | 117125 RA Cartilage Rep2 | 2.3 | 4.7 |
| Crohns-F | | | | | |
| 112725 Crohns-M | 3.2 | 0.0 | 113492 Bone2 RA | 42.3 | 26.8 |
| 112387 Match Control | 2.1 | 8.0 | 113493 Synovium2 RA | 16.6 | 3.7 |
| Crohns-M | | | | | |
| 112378 Crohns-M | 0.0 | 0.0 | 113494 Syn Fluid Cells RA | 31.2 | 23.0 |
| 112390 Match Control | 28.1 | 32.5 | 113499 Cartilage4 RA | 20.9 | 20.0 |
| Crohns-M | | | | | |
| 112726 Crohns-M | 23.2 | 25.3 | 113500 Bone4 RA | 22.8 | 39.5 |
| 112731 Match Control | 20.0 | 15.3 | 113501 Synovium4 RA | 26.6 | 24.8 |
| Crohns-M | | | | | |
| 112380 Ulcer Col-F | 11.3 | 20.7 | 113502 Syn Fluid Cells4 RA | 26.2 | 19.2 |
| 12734 Match Control Ulcer | 82.9 | 43.2 | 113495 Cartilage3 RA | 22.4 | 16.5 |
| Col-F | | | | | |
| 112384 Ulcer Col-F | 24.7 | 25.9 | 113496 Bone3 RA | 13.0 | 18.4 |
| 112737 Match Control Ulcer | 8.3 | 3.7 | 113497 Synovium3 RA | 7.0 | 9.9 |
| Col-F | | | | | |
| 112386 Ulcer Col-F | 2.2 | 3.6 | 113498 Syn Fluid Cells3 RA | 25.9 | 27.4 |
| 112738 Match Control Ulcer | 10.5 | 4.1 | 117106 Normal Cartilage Rep20 | 6.9 | 1.9 |
| Col-F | | | | | |
| 112381 Ulcer Col-M | 1.5 | 0.0 | 113663 Bone3 Normal | 0.0 | 0.0 |
| 112735 Match Control Ulcer | 11.7 | 4.5 | 113664 Synovium3 Normal | 0.0 | 0.0 |
| Col-M | | | | | |
| 112382 Ulcer Col-M | 25.5 | 16.6 | 113665 Syn Fluid Cells3 Normal | 1.2 | 0.0 |
| 112394 Match Control Ulcer | 1.3 | 2.9 | 117107 Normal Cartilage Rep22 | 12.9 | 0.0 |
| Col-M | | | | | |
| 112383 Ulcer Col-M | 12.2 | 11.7 | 113667 Bone4 Normal | 50.7 | 45.4 |
| 112736 Match Control Ulcer | 15.7 | 5.6 | 113668 Synovium4 Normal | 50.0 | 64.6 |
| Col-M | | | | | |
| 112423 Psoriasis-F | 21.2 | 18.2 | 113669 Syn Fluid Cells4 Normal | 100.0 | 100.0 |

TABLE ACC
CNS_neurodegeneration_v1.0
Column A - Rel. Exp. (%) Ag2695, Run 209751330
Column B - Rel. Exp. (%) Ag2695, Run 219966626

| Tissue Name | A | B | Tissue Name | A | B |
|---|---|---|---|---|---|
| AD 1 Hippo | 24.8 | 15.5 | AH3 4624 | 100.0 | 47.6 |
| AD 2 Hippo | 37.6 | 34.4 | AH3 4640 | 58.6 | 63.3 |
| AD 3 Hippo | 27.0 | 19.1 | AD 1 Occipital Ctx | 20.9 | 31.0 |
| AD 4 Hippo | 28.5 | 20.4 | AD 2 Occipital Ctx | 7.8 | 16.7 |
| | | | (Missing) | | |
| AD 5 Hippo | 40.3 | 43.2 | AD 3 Occipital Ctx | 31.9 | 19.1 |
| AD 6 Hippo | 15.0 | 13.4 | AD 4 Occipital Ctx | 51.8 | 40.6 |
| Control 2 Hippo | 18.3 | 20.9 | AD 5 Occipital Ctx | 12.9 | 24.3 |
| Control 4 Hippo | 56.3 | 31.9 | AD 5 Occipital Ctx | 35.4 | 32.1 |
| Control (Path) | 43.2 | 20.9 | Control 1 | 100.0 | 97.3 |
| 3 Hippo | | | Occipital Ctx | | |
| AD 1 Temporal Ctx | 27.7 | 39.5 | Control 2 | 44.1 | 37.9 |
| | | | Occipital Ctx | | |
| AD 2 Temporal Ctx | 21.9 | 35.8 | Control 3 | 50.0 | 49.0 |
| | | | Occipital Ctx | | |
| AD 3 Temporal Ctx | 13.0 | 31.2 | Control 4 | 62.9 | 22.8 |
| | | | Occipital Ctx | | |
| AD 4 Temporal Ctx | 37.6 | 45.4 | Control (Path) 1 | 35.6 | 41.8 |
| | | | Occipital Ctx | | |
| AD 5 Inf Temporal | 32.3 | 44.1 | Control (Path) 2 | 67.4 | 50.0 |
| Ctx | | | Occipital Ctx | | |
| AD 5 Sup Temporal | 31.4 | 26.6 | Control (Path) 3 | 99.3 | 72.7 |
| Ctx | | | Occipital Ctx | | |
| AD 6 Inf Temporal | 16.8 | 15.3 | Control (Path) 4 | 88.9 | 90.8 |
| Ctx | | | Occipital Ctx | | |
| AD 6 Sup Temporal | 18.2 | 18.0 | Control 1 | 97.3 | 94.0 |
| Ctx | | | Parietal Ctx | | |
| Control 1 Temporal | 88.3 | 100.0 | Control 2 | 30.4 | 27.0 |
| Ctx | | | Parietal Ctx | | |
| Control 2 Temporal | 36.3 | 36.1 | Control 3 | 25.0 | 14.4 |
| Ctx | | | Parietal Ctx | | |
| Control 3 Temporal | 32.1 | 43.2 | Control (Path) 1 | 46.3 | 33.4 |
| Ctx | | | Parietal Ctx | | |
| Control 3 Temporal | 32.3 | 62.4 | Control (Path) 2 | 66.0 | 48.3 |
| Ctx | | | Parietal Ctx | | |
| AH3 3975 | 48.6 | 45.4 | Control (Path) 3 | 58.2 | 80.1 |
| | | | Parietal Ctx | | |
| AH3 3954 | 44.1 | 41.8 | Control (Path) 4 | 52.5 | 54.0 |
| | | | Parietal Ctx | | |

TABLE ACD
Panel 1.3D
Column A - Rel. Exp. (%) Ag2695, Run 153140732
Column B - Rel. Exp. (%) Ag2695, Run 153830081

| Tissue Name | A | B | Tissue Name | A | B |
|---|---|---|---|---|---|
| Liver adenocarcinoma | 0.0 | 0.0 | Kidney (fetal) | 0.0 | 3.9 |
| Pancreas | 2.1 | 4.2 | Renal ca. 786-0 | 0.0 | 0.0 |
| Pancreatic ca. CAPAN 2 | 0.0 | 0.0 | Renal ca. A498 | 0.0 | 1.9 |
| Adrenal gland | 67.4 | 100.0 | Renal ca. RXF 393 | 0.0 | 0.0 |
| Thyroid | 5.1 | 2.0 | Renal ca. ACHN | 0.0 | 0.0 |
| Salivary gland | 5.1 | 3.5 | Renal ca. UO-31 | 0.0 | 0.0 |
| Pituitary gland | 1.0 | 4.9 | Renal ca. TK-10 | 0.0 | 0.0 |
| Brain (fetal) | 3.5 | 0.0 | Liver | 1.5 | 3.9 |
| Brain (whole) | 20.3 | 16.0 | Liver (fetal) | 0.0 | 2.5 |
| Brain (amygdala) | 22.2 | 12.5 | Liver ca. (hepatoblast) HepG2 | 0.0 | 0.0 |
| Brain (cerebellum) | 2.1 | 4.5 | Lung | 18.8 | 16.2 |
| Brain (hippocampus) | 61.1 | 35.1 | Lung (fetal) | 3.7 | 3.7 |
| Brain (substantia nigra) | 9.2 | 8.8 | Lung ca. (small cell) LX-1 | 0.0 | 0.0 |
| Brain (thalamus) | 8.7 | 18.6 | Lung ca. (small cell) NCI-H69 | 0.0 | 0.0 |
| Cerebral Cortex | 45.1 | 53.2 | Lung ca. (s. cell var.) SHP-77 | 0.0 | 0.0 |
| Spinal cord | 11.3 | 8.0 | Lung ca. (large cell) NCI-H460 | 0.0 | 0.0 |
| glio/astro U87-MG | 0.0 | 0.0 | Lung ca. (non-sm. cell) A549 | 0.0 | 0.0 |
| glio/astro U-118-MG | 4.2 | 0.0 | Lung ca. (non-s. cell) NCI-H23 | 0.0 | 1.8 |
| astrocytoma SW1783 | 0.0 | 0.0 | Lung ca. (non-s. cell) HOP-62 | 0.0 | 0.0 |
| neuro*; met SK-N-AS | 0.0 | 0.0 | Lung ca. (non-s. cl) NCI-H522 | 0.0 | 0.0 |
| astrocytoma SF-539 | 9.0 | 0.0 | Lung ca. (squam.) SW 900 | 0.0 | 0.0 |
| astrocytoma SNB-75 | 0.0 | 0.0 | Lung ca. (squam.) NCI-H596 | 2.3 | 0.0 |
| glioma SNB-19 | 0.0 | 2.9 | Mammary gland | 5.2 | 9.0 |
| glioma U251 | 0.0 | 0.0 | Breast ca.* (pl. ef) MCF-7 | 0.0 | 0.0 |
| glioma SF-295 | 0.0 | 2.0 | Breast ca.* (pl. ef) MDA-MB- | 0.0 | 0.0 |
| | | | 231 | | |
| Heart (Fetal) | 17.4 | 16.5 | Breast ca.* (pl. ef) T47D | 0.0 | 0.0 |
| Heart | 41.5 | 33.0 | Breast ca. BT-549 | 0.0 | 0.0 |
| Skeletal muscle (Fetal) | 8.6 | 5.2 | Breast ca. MDA-N | 0.0 | 0.0 |
| Skeletal muscle | 0.0 | 0.0 | Ovary | 16.2 | 9.1 |
| Bone marrow | 3.5 | 11.7 | Ovarian ca. OVCAR-3 | 0.0 | 0.0 |
| Thymus | 1.4 | 6.8 | Ovarian ca. OVCAR-4 | 0.0 | 0.0 |
| Spleen | 100.0 | 59.0 | Ovarian ca. OVCAR-5 | 0.0 | 0.0 |
| Lymph node | 32.3 | 26.8 | Ovarian ca. OVCAR-8 | 0.0 | 0.0 |
| Colorectal | 17.9 | 20.2 | Ovarian ca. IGROV-1 | 0.0 | 0.0 |
| Stomach | 9.5 | 0.0 | Ovarian ca. (ascites) SK-OV-3 | 0.0 | 0.0 |
| Small intestine | 13.4 | 39.0 | Uterus | 6.5 | 4.3 |
| Colon ca. SW480 | 0.0 | 0.0 | Placenta | 49.0 | 32.5 |
| Colon ca.* SW620 (SW480 met) | 0.0 | 0.0 | Prostate | 15.0 | 2.2 |
| Colon ca. HT29 | 0.0 | 1.9 | Prostate ca.* (bone met) PC-3 | 0.0 | 0.0 |
| Colon ca. HCT-116 | 0.0 | 0.0 | Testis | 2.4 | 1.0 |
| Colon ca. CaCo-2 | 0.0 | 0.0 | Melanoma Hs688(A).T | 0.0 | 0.0 |
| CC Well to Mod Diff | 11.0 | 3.4 | Melanoma* (met) Hs688(B).T | 0.0 | 0.0 |
| (ODO3866) | | | | | |
| Colon ca. HCC-2998 | 0.6 | 0.0 | Melanoma UACC-62 | 0.0 | 0.0 |
| Gastric ca. (liver met) NCI-N87 | 0.0 | 1.9 | Melanoma M14 | 0.0 | 0.0 |
| Bladder | 0.0 | 2.4 | Melanoma LOX IMVI | 0.0 | 0.0 |
| Trachea | 4.2 | 0.0 | Melanoma* (met) SK-MEL-5 | 14.0 | 4.3 |
| Kidney | 2.4 | 0.0 | Adipose | 15.5 | 16.7 |

TABLE ACE
Panel 2D
Column A - Rel. Exp. (%) Ag2695, Run 153140795
Column B - Rel. Exp. (%) Ag2695, Run 153789847

| Tissue Name | A | B | Tissue Name | A | B |
|---|---|---|---|---|---|
| Normal Colon | 3.8 | 3.3 | Kidney Margin 8120608 | 1.4 | 0.5 |
| CC Well to Mod Diff (ODO3866) | 1.5 | 0.6 | Kidney Cancer 8120613 | 0.1 | 0.1 |
| CC Margin (ODO3866) | 2.7 | 0.5 | Kidney Margin 8120614 | 0.6 | 0.0 |
| CC Gr.2 rectosigmoid (ODO3868) | 0.1 | 0.2 | Kidney Cancer 9010320 | 4.0 | 2.5 |
| CC Margin (ODO3868) | 0.9 | 0.3 | Kidney Margin 9010321 | 0.4 | 1.1 |
| CC Mod Diff (ODO3920) | 0.6 | 0.2 | Normal Uterus | 0.4 | 0.3 |
| CC Margin (ODO3920) | 0.5 | 0.3 | Uterine Cancer 064011 | 1.2 | 0.9 |
| CC Gr.2 ascend colon (ODO3921) | 3.2 | 2.2 | Normal Thyroid | 0.6 | 0.8 |
| CC Margin (ODO3921) | 0.8 | 0.6 | Thyroid Cancer | 7.3 | 5.7 |
| CC from Partial Hepatectomy | 1.6 | 2.0 | Thyroid Cancer A302152 | 7.1 | 8.9 |
| (ODO4309) Mets | | | | | |
| Liver Margin (ODO4309) | 1.3 | 1.5 | Thyroid Margin A302153 | 1.3 | 1.1 |
| Colon mets to lung (OD04451-01) | 0.3 | 1.2 | Normal Breast | 1.1 | 1.8 |
| Lung Margin (OD04451-02) | 2.9 | 1.7 | Breast Cancer | 0.2 | 1.3 |
| Normal Prostate 6546-1 | 2.0 | 0.8 | Breast Cancer (OD04590-01) | 2.2 | 2.0 |
| Prostate Cancer (OD04410) | 1.9 | 1.7 | Breast Cancer Mets | | |
| | | | (OD04590-03) | 6.7 | 8.5 |
| Prostate Margin (OD04410) | 3.5 | 3.9 | Breast Cancer Metastasis | 2.7 | 2.7 |
| Prostate Cancer (OD04720-01) | 1.7 | 0.5 | Breast Cancer | 0.6 | 0.6 |
| Prostate Margin (OD04720-02) | 5.0 | 4.4 | Breast Cancer | 0.7 | 0.6 |
| Normal Lung | 6.9 | 7.8 | Breast Cancer 9100266 | 0.1 | 0.3 |
| Lung Met to Muscle (ODO4286) | 2.2 | 2.0 | Breast Margin 9100265 | 0.2 | 0.5 |
| Muscle Margin (ODO4286) | 0.3 | 0.5 | Breast Cancer A209073 | 2.3 | 1.7 |
| Lung Malignant Cancer | 2.4 | 3.7 | Breast Margin A209073 | 0.4 | 0.3 |
| (OD03126) | | | | | |
| Lung Margin (OD03126) | 6.9 | 6.7 | Normal Liver | 0.6 | 0.2 |
| Lung Cancer (OD04404) | 5.5 | 2.1 | Liver Cancer | 0.8 | 0.0 |
| Lung Margin (OD04404) | 1.6 | 1.9 | Liver Cancer 1025 | 0.2 | 0.2 |
| Lung Cancer (OD04565) | 0.6 | 0.5 | Liver Cancer 1026 | 0.8 | 0.6 |
| Lung Margin (OD04565) | 1.0 | 2.0 | Liver Cancer 6004-T | 0.4 | 0.5 |
| Lung Cancer (OD04237-01) | 4.5 | 3.8 | Liver Tissue 6004-N | 0.9 | 0.3 |
| Lung Margin (OD04237-02) | 3.9 | 5.2 | Liver Cancer 6005-T | 0.2 | 0.2 |
| Ocular Mel Met to Liver | 0.0 | 0.2 | Liver Tissue 6005-N | 0.3 | 0.3 |
| (ODO4310) | | | | | |
| Liver Margin (ODO4310) | 1.7 | 0.7 | Normal Bladder | 0.9 | 1.5 |
| Melanoma Metastasis | 100.0 | 100.0 | Bladder Cancer | 0.1 | 0.4 |
| Lung Margin (OD04321) | 7.3 | 6.5 | Bladder Cancer | 0.5 | 1.2 |
| Normal Kidney | 6.4 | 5.0 | Bladder Cancer (OD04718- | 1.8 | 1.2 |
| | | | 01) | | |
| Kidney Ca, Nuclear grade 2 | 34.2 | 33.9 | Bladder Normal Adjacent | 2.2 | 1.9 |
| (OD04338) | | | (OD04718-03) | | |
| Kidney Margin (OD04338) | 5.2 | 5.6 | Normal Ovary | 0.6 | 0.2 |
| Kidney Ca Nuclear grade 1/2 | 1.4 | 0.8 | Ovarian Cancer | 2.9 | 1.7 |
| (OD04339) | | | | | |
| Kidney Margin (OD04339) | 0.9 | 2.0 | Ovarian Cancer (OD04768- | 14.4 | 10.8 |
| | | | 07) | | |
| Kidney Ca, Clear cell type | 8.5 | 10.3 | Ovary Margin (OD04768-08) | 1.4 | 1.0 |
| (OD04340) | | | | | |
| Kidney Margin (OD04340) | 11.3 | 6.6 | Normal Stomach | 0.6 | 1.5 |
| Kidney Ca, Nuclear grade 3 | 1.4 | 1.9 | Gastric Cancer 9060358 | 0.5 | 0.7 |
| (OD04348) | | | | | |
| Kidney Margin (OD04348) | 5.4 | 7.3 | Stomach Margin 9060359 | 0.6 | 0.6 |
| Kidney Cancer (OD04622-01) | 49.0 | 63.7 | Gastric Cancer 9060395 | 1.1 | 1.2 |
| Kidney Margin (OD04622-03) | 0.7 | 1.2 | Stomach Margin 9060394 | 1.2 | 0.4 |
| Kidney Cancer (OD04450-01) | 1.4 | 1.4 | Gastric Cancer 9060397 | 0.9 | 0.9 |
| Kidney Margin (OD04450-03) | 5.7 | 4.6 | Stomach Margin 9060396 | 1.7 | 1.1 |
| Kidney Cancer 8120607 | 0.2 | 0.0 | Gastric Cancer 064005 | 3.0 | 2.6 |

TABLE ACF
Panel 4D
Column A - Rel. Exp. (%) Ag2695, Run 153140809
Column B - Rel. Exp. (%) Ag2695, Run 153766369

| Tissue Name | A | B | Tissue Name | A | B |
|---|---|---|---|---|---|
| Secondary Th1 act | 1.0 | 1.6 | HUVEC IL-1beta | 0.0 | 0.0 |
| Secondary Th2 act | 21.0 | 29.5 | HUVEC IFN gamma | 0.0 | 0.0 |
| Secondary Tr1 act | 5.0 | 10.8 | HUVEC TNFalpha + IFN gamma | 0.0 | 0.0 |
| Secondary Th1 rest | 0.6 | 3.8 | HUVEC TNFalpha + IL4 | 0.0 | 0.0 |
| Secondary Th2 rest | 13.9 | 15.4 | HUVEC IL-11 | 0.0 | 0.0 |
| Secondary Tr1 rest | 6.1 | 5.0 | Lung Microvascular EC none | 0.0 | 0.0 |
| Primary Th1 act | 0.0 | 2.6 | Lung Microvascular EC TNFalpha + | 0.0 | 0.0 |
| | | | IL-1beta | | |
| Primary Th2 act | 39.0 | 54.3 | Microvascular Dermal EC none | 0.0 | 0.0 |
| Primary Tr1 act | 10.7 | 21.6 | Microsvasular Dermal EC TNFalpha + | 0.0 | 0.0 |
| | | | IL-1beta | | |
| Primary Th1 rest | 21.0 | 24.7 | Bronchial epithelium TNFalpha + | 0.0 | 0.0 |
| | | | IL1beta | | |
| Primary Th2 rest | 26.1 | 21.2 | Small airway epithelium none | 0.0 | 0.0 |
| Primary Tr1 rest | 11.7 | 19.1 | Small airway epithelium TNFalpha + | 0.0 | 0.0 |
| | | | IL-1beta | | |
| CD45RA CD4 lymphocyte | 9.6 | 7.1 | Coronery artery SMC rest | 0.0 | 0.0 |
| act | | | | | |
| CD45RO CD4 lymphocyte | 15.6 | 17.3 | Coronery artery SMC TNFalpha + | 0.0 | 0.0 |
| act | | | IL-1beta | | |
| CD8 lymphocyte act | 11.7 | 9.3 | Astrocytes rest | 0.0 | 0.5 |
| Secondary CD8 lymphocyte | 8.4 | 9.0 | Astrocytes TNFalpha + IL-1beta | 0.0 | 0.0 |
| rest | | | | | |
| Secondary CD8 lymphocyte | 22.2 | 18.3 | KU-812 (Basophil) rest | 0.5 | 2.4 |
| act | | | | | |
| CD4 lymphocyte none | 18.6 | 9.1 | KU-812 (Basophil) PMA/ionomycin | 2.9 | 1.7 |
| 2ry Th1/Th2/Tr1 anti-CD95 | 3.8 | 7.6 | CCD1106 (Keratinocytes) none | 0.0 | 0.7 |
| CH11 | | | | | |
| LAK cells rest | 43.2 | 32.5 | CCD1106 (Keratinocytes) TNFalpha + | 0.0 | 0.0 |
| | | | IL-1beta | | |
| LAK cells IL-2 | 15.9 | 18.0 | Liver cirrhosis | 4.8 | 6.3 |
| LAK cells IL-2 + IL-12 | 32.3 | 30.8 | Lupus kidney | 0.6 | 0.0 |
| LAK cells IL-2 + IFN | 62.9 | 57.8 | NCI-H292 none | 0.0 | 0.0 |
| gamma | | | | | |
| LAK cells IL-2 + IL-18 | 39.2 | 52.1 | NCI-H292 IL-4 | 0.0 | 0.0 |
| LAK cells PMA/ionomycin | 43.5 | 52.9 | NCI-H292 IL-9 | 0.0 | 0.0 |
| NK Cells IL-2 rest | 37.4 | 35.6 | NCI-H292 IL-13 | 0.0 | 0.0 |
| Two Way MLR 3 day | 26.1 | 23.0 | NCI-H292 IFN gamma | 0.0 | 0.0 |
| Two Way MLR 5 day | 17.0 | 10.7 | HPAEC none | 0.0 | 0.5 |
| Two Way MLR 7 day | 2.1 | 7.4 | HPAEC TNFalpha + IL-1 beta | 0.0 | 0.0 |
| PBMC rest | 19.9 | 28.3 | Lung fibroblast none | 0.0 | 0.0 |
| PBMC PWM | 28.3 | 31.4 | Lung fibroblast TNFalpha + IL-1 | 0.0 | 0.0 |
| | | | beta | | |
| PBMC PHA-L | 10.9 | 14.4 | Lung fibroblast IL-4 | 0.0 | 0.0 |
| Ramos (B cell) none | 0.0 | 0.1 | Lung fibroblast IL-9 | 0.0 | 0.0 |
| Ramos (B cell) ionomycin | 0.0 | 0.0 | Lung fibroblast IL-13 | 0.0 | 0.0 |
| B lymphocytes PWM | 9.0 | 9.5 | Lung fibroblast IFN gamma | 0.0 | 0.0 |
| B lymphocytes CD40L and | 9.5 | 11.1 | Dermal fibroblast CCD1070 rest | 0.0 | 0.0 |
| IL-4 | | | | | |
| EOL-1 dbcAMP | 1.1 | 0.0 | Dermal fibroblast CCD1070 TNF | 17.1 | 20.3 |
| | | | alpha | | |
| EOL-1 dbcAMP | 3.7 | 0.6 | Dermal fibroblast CCD1070 IL-1 | 0.0 | 0.0 |
| PMA/ionomycin | | | beta | | |
| Dendritic cells none | 3.7 | 10.3 | Dermal fibroblast IFN gamma | 0.0 | 0.0 |
| Dendritic cells LPS | 6.1 | 8.1 | Dermal fibroblast IL-4 | 1.1 | 0.0 |
| Dendritic cells anti-CD40 | 4.9 | 3.6 | IBD Colitis 2 | 1.1 | 0.0 |
| Monocytes rest | 100.0 | 100.0 | IBD Crohn's | 2.2 | 3.5 |
| Monocytes LPS | 0.0 | 0.3 | Colon | 21.5 | 15.6 |
| Macrophages rest | 0.6 | 3.7 | Lung | 17.6 | 14.4 |
| Macrophages LPS | 1.0 | 3.1 | Thymus | 11.8 | 7.1 |
| HUVEC none | 0.0 | 0.0 | Kidney | 33.7 | 20.3 |
| HUVEC starved | 0.0 | 0.0 | | | |

AI_comprehensive panel_v1.0 Summary: Ag2695 The highest expression of this gene was detected in synovial fluid cells. Low expression of this gene is also seen in orthoarhritis bone, cartilage, synovium, RA bone, normal lung and a psoriasis sample. Therapeutic modulation of this gene, expressed protein and/or use of antibodies or small molecule drugs targeting the gene or gene product are useful in the treatment of orthoarthritis, rheumatoid arthritis and psoriasis.

CNS_neurodegeneration_v1.0 Summary: Ag2695 This gene was down-regulated in the temporal cortex of Alzheimer's disease patients. Up-regulation of this gene or its protein product, or treatment with specific agonists for this protein encoded by this gene is useful in reversing the dementia/memory loss associated with this disease and neuronal death.

Panel 1.3D Summary: Ag2695 Highest expression of this gene was seen in adrenal gland and spleen (CTS=31.7). Significant expression of this gene is seen mainly in the normal tissues including brain, lymphnode, heart, gastrointestinal tract, lung, ovary, placenta and adipose tissue. Expression of this gene was low or undetectable in any of the cancer cell lines. Therapeutic modulation of this gene, expressed protein and/or use of antibodies or small molecule drugs targeting the gene or gene product are useful in the treatment of cancer, metabolic and CNS disorders.

Panel 2D Summary: Ag2695 The expression of this gene was detected in metastatic melanoma (CTs=26-27.8). High to moderate expression of this gene was also seen in normal and cancer samples from colon, lung, prostate, liver, prostate, thyroid, uterus, breast, ovary and stomach. Expression of this gene is upregulated in ovarian, thyroid and kidney cancers compared to corresponding normal adjacent normal tissues. Therapeutic modulation of this gene, expressed protein and/or use of antibodies or small molecule drugs targeting the gene or gene product are useful in the treatment of ovarian, thyroid and kidney cancers.

Panel 4D Summary: Ag2695 Highest expression of this gene was seen in resting monocytes (CT=29.6). This gene was expressed by T lymphocytes prepared under a number of conditions at moderate levels and is expressed at significant levels in treated and untreated dendritic cells, LAK cells, PBMC, activated B lymphocytes, activated dermal fibroblasts, liver cirrhosis sample and normal tissues represented by colon, lung, thymus and kidney. Dendritic cells are powerful antigen-presenting cells (APC) whose function is pivotal in the initiation and maintenance of normal immune responses. Autoimmunity and inflammation may also be reduced by suppression of this function. Therapeutic modulation of this gene, expressed protein and/or use of antibodies or small molecule drugs targeting the gene or gene product are useful in the treatment of autoimmune and inflammatory diseases, such as lupus erythematosus, Crohn's disease, ulcerative colitis, multiple sclerosis, chronic obstructive pulmonary disease, asthma, emphysema, rheumatoid arthritis, or psoriasis.

AD. CG54479-05: Hepatocyte Growth Factor-Like Protein Precursor.

Expression of gene CG54479-05 was assessed using the primer-probe sets Ag3086 and Ag3797, described in Tables ADA and ADB. Results of the RTQ-PCR runs are shown in Tables ADC, ADD, ADE and ADF.

TABLE ADA
Probe Name Ag3086

| | | | Start | SEQ ID |
|---|---|---|---|---|
| Primers | Sequences | Length | Position | No |
| Forward | 5′-ggaccccattcgactactgt-3′ | 20 | 1229 | 1277 |
| Probe | TET-5′-ctgatgaccagccgccatcaatc-3′- | 23 | 1265 | 1278 |
| | TAMRA | | | |
| Reverse | 5′-ttctcaaactgcacctggtc-3′ | 20 | 1300 | 1279 |

TABLE ADB
Probe Name Ag3797

| | | | Start | SEQ ID |
|---|---|---|---|---|
| Primers | Sequences | Length | Position | No |
| Forward | 5′-tctggacgacaactattgcc-3′ | 20 | 621 | 1280 |
| Probe | TET-5′-atggtgctacactacggatccgcag-3′- | 25 | 666 | 1281 |
| | TAMRA | | | |
| Reverse | 5′-gtcacagaattctcgctcga-3′ | 20 | 692 | 1282 |

TABLE ADC
General_screening_panel_v1.4
Column A - Rel. Exp.(%) Ag3797, Run 217309613

| Tissue Name | A | Tissue Name | A |
|---|---|---|---|
| Adipose | 1.4 | Renal ca. TK-10 | 28.9 |
| Melanoma* | 0.4 | Bladder | 8.4 |
| Hs688(A).T | | | |
| Melanoma* | 0.5 | Gastric ca. (liver met.) NCI-N87 | 2.7 |
| Hs688(B).T | | | |
| Melanoma* M14 | 0.3 | Gastric ca. KATO III | 1.4 |
| Melanoma* LOXIMVI | 0.3 | Colon ca. SW-948 | 1.0 |
| Melanoma* | 0.5 | Colon ca. SW480 | 3.9 |
| SK-MEL-5 | | | |
| Squamous cell | 0.8 | Colon ca.* (SW480 met) SW620 | 1.2 |
| carcinoma SCC-4 | | | |
| Testis Pool | 2.0 | Colon ca. HT29 | 0.2 |
| Prostate ca.* PC-3 | 1.5 | Colon ca. HCT-116 | 4.3 |
| (bone met) | | | |
| Prostate Pool | 1.8 | Colon ca. CaCo-2 | 11.5 |
| Placenta | 1.7 | Colon cancer tissue | 2.8 |
| Uterus Pool | 0.5 | Colon ca. SW1116 | 2.9 |
| Ovarian ca. OVCAR-3 | 1.0 | Colon ca. Colo-205 | 0.5 |
| Ovarian ca. SK-OV-3 | 0.8 | Colon ca. SW-48 | 0.2 |
| Ovarian ca. OVCAR-4 | 0.3 | Colon Pool | 1.6 |
| Ovarian ca. OVCAR-5 | 6.4 | Small Intestine Pool | 2.0 |
| Ovarian ca. IGROV-1 | 4.6 | Stomach Pool | 1.9 |
| Ovarian ca. OVCAR-8 | 2.9 | Bone Marrow Pool | 0.4 |
| Ovary | 1.9 | Fetal Heart | 0.8 |
| Breast ca. MCF-7 | 2.3 | Heart Pool | 0.7 |
| Breast ca. | 2.2 | Lymph Node Pool | 1.7 |
| MDA-MB-231 | | | |
| Breast ca. BT 549 | 3.0 | Fetal Skeletal Muscle | 0.7 |
| Breast ca. T47D | 18.6 | Skeletal Muscle Pool | 1.1 |
| Breast ca. MDA-N | 0.7 | Spleen Pool | 2.5 |
| Breast Pool | 1.5 | Thymus Pool | 2.4 |
| Trachea | 1.2 | CNS cancer (glio/astro) U87-MG | 2.7 |
| Lung | 0.4 | CNS cancer (glio/astro) U-118-MG | 3.1 |
| Fetal Lung | 2.3 | CNS cancer (neuro; met) SK-N-AS | 2.1 |
| Lung ca. NCI-N417 | 0.2 | CNS cancer (astro) SF-539 | 0.6 |
| Lung ca. LX-1 | 3.3 | CNS cancer (astro) SNB-75 | 1.8 |
| Lung ca. NCI-H146 | 0.5 | CNS cancer (glio) SNB-19 | 4.1 |
| Lung ca. SHP-77 | 2.5 | CNS cancer (glio) SF-295 | 2.1 |
| Lung ca. A549 | 0.6 | Brain (Amygdala) Pool | 0.9 |
| Lung ca. NCI-H526 | 0.6 | Brain (cerebellum) | 1.9 |
| Lung ca. NCI-H23 | 3.7 | Brain (fetal) | 2.8 |
| Lung ca. NCI-H460 | 0.9 | Brain (Hippocampus) Pool | 1.0 |
| Lung ca. HOP-62 | 1.2 | Cerebral Cortex Pool | 0.7 |
| Lung ca. NCI-H522 | 1.7 | Brain (Substantia nigra) Pool | 0.9 |
| Liver | 26.6 | Brain (Thalamus) Pool | 1.0 |
| Fetal Liver | 45.7 | Brain (whole) | 1.6 |
| Liver ca. HepG2 | 100.0 | Spinal Cord Pool | 1.7 |
| Kidney Pool | 1.7 | Adrenal Gland | 3.1 |
| Fetal Kidney | 11.1 | Pituitary gland Pool | 1.7 |
| Renal ca. 786-0 | 1.0 | Salivary Gland | 1.0 |
| Renal ca. A498 | 0.3 | Thyroid (female) | 2.8 |
| Renal ca. ACHN | 1.4 | Pancreatic ca. CAPAN2 | 0.8 |
| Renal ca. UO-31 | 1.8 | Pancreas Pool | 7.5 |

TABLE ADD
Panel 1.3D
Column A - Rel. Exp.(%) Ag3086, Run 165552724

| Tissue Name | A | Tissue Name | A |
|---|---|---|---|
| Liver adenocarcinoma | 0.7 | Kidney (fetal) | 31.2 |
| Pancreas | 17.9 | Renal ca. 786-0 | 0.2 |
| Pancreatic ca. | 0.6 | Renal ca. A498 | 0.5 |
| CAPAN 2 | | | |
| Adrenal gland | 2.7 | Renal ca. RXF 393 | 0.7 |
| Thyroid | 3.3 | Renal ca. ACHN | 0.8 |
| Salivary gland | 1.2 | Renal ca. UO-31 | 0.4 |
| Pituitary gland | 3.6 | Renal ca. TK-10 | 0.2 |
| Brain (fetal) | 3.2 | Liver | 94.6 |
| Brain (whole) | 3.4 | Liver (fetal) | 100.0 |
| Brain (amygdala) | 2.1 | Liver ca. (hepatoblast) HepG2 | 58.6 |
| Brain (cerebellum) | 1.5 | Lung | 2.8 |
| Brain (hippocampus) | 3.0 | Lung (fetal) | 12.9 |
| Brain (substantia | 1.7 | Lung ca. (small cell) LX-1 | 1.3 |
| nigra) | | | |
| Brain (thalamus) | 3.1 | Lung ca. (small cell) NCI-H69 | 0.2 |
| Cerebral Cortex | 0.9 | Lung ca. (s. cell var.) SHP-77 | 1.2 |
| Spinal cord | 2.9 | Lung ca. (large cell) NCI-H460 | 1.4 |
| glio/astro U87-MG | 0.7 | Lung ca. (non-sm. cell) A549 | 0.2 |
| glio/astro U-118-MG | 0.9 | Lung ca. (non-s. cell) NCI-H23 | 0.9 |
| astrocytoma SW1783 | 0.4 | Lung ca. (non-s. cell) HOP-62 | 0.5 |
| neuro*; met SK-N-AS | 0.7 | Lung ca. (non-s. cl) NCI-H522 | 0.6 |
| astrocytoma SF-539 | 0.5 | Lung ca. (squam.) SW 900 | 0.4 |
| astrocytoma SNB-75 | 1.2 | Lung ca. (squam.) NCI-H596 | 0.5 |
| glioma SNB-19 | 1.6 | Mammary gland | 3.1 |
| glioma U251 | 2.4 | Breast ca.* (pl.ef) MCF-7 | 0.7 |
| glioma SF-295 | 0.7 | Breast ca.* (pl.ef) MDA-MB-231 | 0.7 |
| Heart (Fetal) | 0.6 | Breast ca.* (pl.ef) T47D | 2.7 |
| Heart | 0.5 | Breast ca. BT-549 | 0.7 |
| Skeletal muscle (Fetal) | 0.2 | Breast ca. MDA-N | 0.1 |
| Skeletal muscle | 1.4 | Ovary | 0.4 |
| Bone marrow | 2.0 | Ovarian ca. OVCAR-3 | 0.6 |
| Thymus | 1.2 | Ovarian ca. OVCAR-4 | 0.4 |
| Spleen | 4.0 | Ovarian ca. OVCAR-5 | 0.4 |
| Lymph node | 3.1 | Ovarian ca. OVCAR-8 | 0.7 |
| Colorectal | 1.6 | Ovarian ca. IGROV-1 | 0.7 |
| Stomach | 10.3 | Ovarian ca. (ascites) SK-OV-3 | 0.1 |
| Small intestine | 29.7 | Uterus | 3.3 |
| Colon ca. SW480 | 0.7 | Placenta | 4.6 |
| Colon ca.* SW620 | 0.2 | Prostate | 2.1 |
| (SW480 met) | | | |
| Colon ca. HT29 | 0.2 | Prostate ca.* (bone met) PC-3 | 0.9 |
| Colon ca. HCT-116 | 1.2 | Testis | 12.5 |
| Colon ca. CaCo-2 | 2.4 | Melanoma Hs688(A).T | 0.1 |
| CC Well to Mod Diff | 2.1 | Melanoma* (met) Hs688(B).T | 0.2 |
| (ODO3866) | | | |
| Colon ca. HCC-2998 | 1.4 | Melanoma UACC-62 | 0.4 |
| Gastric ca. (liver met) | 1.8 | Melanoma M14 | 0.8 |
| NCI-N87 | | | |
| Bladder | 4.5 | Melanoma LOX IMVI | 0.0 |
| Trachea | 2.6 | Melanoma* (met) SK-MEL-5 | 0.4 |
| Kidney | 26.1 | Adipose | 2.2 |

TABLE ADE
Panel 2.2
Column A - Rel. Exp.(%) Ag3086, Run 174268933

| Tissue Name | A | Tissue Name | A |
|---|---|---|---|
| Normal Colon | 1.4 | Kidney Margin (OD04348) | 40.9 |
| Colon cancer (OD06064) | 0.1 | Kidney malignant cancer | 0.4 |
| | | (OD06204B) | |
| Colon Margin (OD06064) | 0.0 | Kidney normal adjacent tissue | 5.1 |
| | | (OD06204E) | |
| Colon cancer (OD06159) | 0.1 | Kidney Cancer (OD04450-01) | 5.8 |
| Colon Margin (OD06159) | 1.4 | Kidney Margin (OD04450-03) | 15.0 |
| Colon cancer (OD06297-04) | 0.0 | Kidney Cancer 8120613 | 0.2 |
| Colon Margin (OD06297-05) | 1.1 | Kidney Margin 8120614 | 6.0 |
| CC Gr.2 ascend colon (ODO3921) | 0.4 | Kidney Cancer 9010320 | 0.4 |
| CC Margin (ODO3921) | 0.2 | Kidney Margin 9010321 | 1.5 |
| Colon cancer metastasis (OD06104) | 0.1 | Kidney Cancer 8120607 | 0.1 |
| Lung Margin (OD06104) | 0.6 | Kidney Margin 8120608 | 3.5 |
| Colon mets to lung (OD04451-01) | 1.3 | Normal Uterus | 0.2 |
| Lung Margin (OD04451-02) | 0.4 | Uterine Cancer 064011 | 0.1 |
| Normal Prostate | 0.6 | Normal Thyroid | 0.5 |
| Prostate Cancer (OD04410) | 0.2 | Thyroid Cancer | 0.3 |
| Prostate Margin (OD04410) | 0.5 | Thyroid Cancer A302152 | 2.1 |
| Normal Ovary | 0.4 | Thyroid Margin A302153 | 0.4 |
| Ovarian cancer (OD06283-03) | 0.2 | Normal Breast | 0.7 |
| Ovarian Margin (OD06283-07) | 1.2 | Breast Cancer | 2.3 |
| Ovarian Cancer | 1.5 | Breast Cancer | 1.9 |
| Ovarian cancer (OD06145) | 0.9 | Breast Cancer (OD04590-01) | 5.1 |
| Ovarian Margin (OD06145) | 1.8 | Breast Cancer Mets (OD04590-03) | 1.3 |
| Ovarian cancer (OD06455-03) | 0.6 | Breast Cancer Metastasis | 0.7 |
| Ovarian Margin (OD06455-07) | 0.2 | Breast Cancer | 0.5 |
| Normal Lung | 0.8 | Breast Cancer 9100266 | 0.2 |
| Invasive poor diff. lung adeno | 0.3 | Breast Margin 9100265 | 0.5 |
| (ODO4945-01 | | | |
| Lung Margin (ODO4945-03) | 1.1 | Breast Cancer A209073 | 0.2 |
| Lung Malignant Cancer (OD03126) | 1.6 | Breast Margin A209073 | 1.4 |
| Lung Margin (OD03126) | 0.3 | Breast cancer (OD06083) | 0.7 |
| Lung Cancer (OD05014A) | 0.5 | Breast cancer node metastasis | 0.2 |
| | | (OD06083) | |
| Lung Margin (OD05014B) | 0.6 | Normal Liver | 28.7 |
| Lung cancer (OD06081) | 0.5 | Liver Cancer 1026 | 7.5 |
| Lung Margin (OD06081) | 1.2 | Liver Cancer 1025 | 45.1 |
| Lung Cancer (OD04237-01) | 0.3 | Liver Cancer 6004-T | 35.8 |
| Lung Margin (OD04237-02) | 1.1 | Liver Tissue 6004-N | 5.1 |
| Ocular Mel Met to Liver (ODO4310) | 0.2 | Liver Cancer 6005-T | 14.8 |
| Liver Margin (ODO4310) | 21.6 | Liver Tissue 6005-N | 65.1 |
| Melanoma Metastasis | 0.2 | Liver Cancer | 100.0 |
| Lung Margin (OD04321) | 0.2 | Normal Bladder | 2.8 |
| Normal Kidney | 5.6 | Bladder Cancer | 0.2 |
| Kidney Ca, Nuclear grade 2 | 20.6 | Bladder Cancer | 0.7 |
| (OD04338) | | | |
| Kidney Margin (OD04338) | 4.5 | Normal Stomach | 2.5 |
| Kidney Ca Nuclear grade ½ | 6.6 | Gastric Cancer 9060397 | 0.0 |
| (OD04339) | | | |
| Kidney Margin (OD04339) | 6.0 | Stomach Margin 9060396 | 0.1 |
| Kidney Ca, Clear cell type (OD04340) | 0.4 | Gastric Cancer 9060395 | 0.1 |
| Kidney Margin (OD04340) | 8.8 | Stomach Margin 9060394 | 0.3 |
| Kidney Ca, Nuclear grade 3 | 0.3 | Gastric Cancer 064005 | 2.8 |
| (OD04348) | | | |

TABLE ADF
Panel 4.1D
Column A - Rel. Exp. (%) Ag3797, Run 170132421
Column B - Rel. Exp. (%) Ag3797, Run 170275745

| Tissue Name | A | B | Tissue Name | A | B |
|---|---|---|---|---|---|
| Secondary Th1 act | 8.9 | 1.3 | HUVEC IL-1beta | 2.4 | 0.1 |
| Secondary Th2 act | 2.6 | 1.3 | HUVEC IFN gamma | 2.6 | 1.2 |
| Secondary Tr1 act | 3.1 | 0.9 | HUVEC TNFalpha + IFN gamma | 0.0 | 0.3 |
| Secondary Th1 rest | 1.5 | 1.6 | HUVEC TNFalpha + IL4 | 1.6 | 0.4 |
| Secondary Th2 rest | 3.3 | 0.7 | HUVEC IL-11 | 2.2 | 0.4 |
| Secondary Tr1 rest | 3.4 | 0.9 | Lung Microvascular EC none | 1.8 | 0.9 |
| Primary Th1 act | 3.2 | 0.3 | Lung Microvascular EC TNFalpha + | 2.2 | 0.3 |
| | | | IL-1beta | | |
| Primary Th2 act | 2.0 | 1.1 | Microvascular Dermal EC none | 1.4 | 0.5 |
| Primary Tr1 act | 2.7 | 1.0 | Microsvasular Dermal EC TNFalpha + | 2.2 | 0.3 |
| | | | IL-1beta | | |
| Primary Th1 rest | 3.2 | 0.4 | Bronchial epithelium TNFalpha + | 17.8 | 0.5 |
| | | | IL1beta | | |
| Primary Th2 rest | 4.5 | 0.0 | Small airway epithelium none | 1.5 | 0.2 |
| Primary Tr1 rest | 2.3 | 0.7 | Small airway epithelium TNFalpha + | 3.0 | 0.6 |
| | | | IL-1beta | | |
| CD45RA CD4 lymphocyte | 2.0 | 0.7 | Coronery artery SMC rest | 1.1 | 0.6 |
| act | | | | | |
| CD45RO CD4 lymphocyte | 1.8 | 2.0 | Coronery artery SMC TNFalpha + IL- | 1.9 | 0.8 |
| act | | | 1beta | | |
| CD8 lymphocyte act | 5.6 | 0.9 | Astrocytes rest | 2.5 | 1.5 |
| Secondary CD8 lymphocyte | 4.6 | 1.1 | Astrocytes TNFalpha + IL-1beta | 0.5 | 1.2 |
| rest | | | | | |
| Secondary CD8 lymphocyte | 1.8 | 0.2 | KU-812 (Basophil) rest | 4.3 | 0.8 |
| act | | | | | |
| CD4 lymphocyte none | 7.2 | 1.3 | KU-812 (Basophil) PMA/ionomycin | 3.0 | 0.6 |
| 2ry Th1/Th2/Trl anti-CD95 | 6.6 | 1.1 | CCD1106 (Keratinocytes) none | 1.6 | 0.9 |
| CH11 | | | | | |
| LAK cells rest | 7.6 | 0.4 | CCD1106 (Keratinocytes) TNFalpha + | 2.4 | 0.0 |
| | | | IL-1beta | | |
| LAK cells IL-2 | 4.8 | 0.3 | Liver cirrhosis | 76.8 | 9.8 |
| LAK cells IL-2 + IL-12 | 5.7 | 0.8 | NCI-H292 none | 5.4 | 4.1 |
| LAK cells IL-2 + IFN gamma | 5.5 | 0.2 | NCI-H292 IL-4 | 10.3 | 0.6 |
| LAK cells IL-2 + IL-18 | 1.6 | 0.4 | NCI-H292 IL-9 | 16.5 | 1.3 |
| LAK cells PMA/ionomycin | 2.7 | 1.3 | NCI-H292 IL-13 | 8.5 | 3.6 |
| NK Cells IL-2 rest | 5.6 | 2.0 | NCI-H292 IFN gamma | 5.8 | 3.4 |
| Two Way MLR 3 day | 4.9 | 2.0 | HPAEC none | 1.2 | 1.0 |
| Two Way MLR 5 day | 0.5 | 0.8 | HPAEC TNFalpha + IL-1 beta | 1.5 | 0.3 |
| Two Way MLR 7 day | 6.0 | 0.2 | Lung fibroblast none | 2.5 | 0.8 |
| PBMC rest | 1.3 | 0.4 | Lung fibroblast TNFalpha + IL-1 beta | 5.7 | 0.9 |
| PBMC PWM | 7.9 | 0.5 | Lung fibroblast IL-4 | 2.9 | 0.4 |
| PBMC PHA-L | 5.1 | 0.5 | Lung fibroblast IL-9 | 2.5 | 0.4 |
| Ramos (B cell) none | 5.7 | 0.6 | Lung fibroblast IL-13 | 2.7 | 1.9 |
| Ramos (B cell) ionomycin | 4.4 | 0.3 | Lung fibroblast IFN gamma | 0.0 | 2.2 |
| B lymphocytes PWM | 1.1 | 0.2 | Dermal fibroblast CCD1070 rest | 6.1 | 3.4 |
| B lymphocytes CD40L and | 4.3 | 0.6 | Dermal fibroblast CCD1070 TNF | 2.1 | 3.8 |
| IL-4 | | | alpha | | |
| EOL-1 dbcAMP | 8.4 | 3.5 | Dermal fibroblast CCD1070 IL-1 beta | 4.0 | 1.5 |
| EOL-1 dbcAMP | 7.2 | 5.1 | Dermal fibroblast IFN gamma | 1.6 | 0.9 |
| PMA/ionomycin | | | | | |
| Dendritic cells none | 3.4 | 1.0 | Dermal fibroblast IL-4 | 1.4 | 1.3 |
| Dendritic cells LPS | 5.5 | 0.5 | Dermal Fibroblasts rest | 2.3 | 1.5 |
| Dendritic cells anti-CD40 | 2.6 | 0.3 | Neutrophils TNFa + LPS | 0.0 | 1.7 |
| Monocytes rest | 1.1 | 0.9 | Neutrophils rest | 0.8 | 0.6 |
| Monocytes LPS | 2.6 | 0.3 | Colon | 21.6 | 6.7 |
| Macrophages rest | 5.2 | 0.2 | Lung | 3.7 | 10.6 |
| Macrophages LPS | 1.4 | 0.3 | Thymus | 11.7 | 27.0 |
| HUVEC none | 1.2 | 0.2 | Kidney | 100.0 | 100.0 |
| HUVEC starved | 3.4 | 0.2 | | | |

General_screening_panel_v1.4 Summary: Ag3797 Highest expression of this gene was detected in liver cancer HepG2 cell line (CT=25.3). High expression of this gene was also seen in fetal and adult liver. Therapeutic modulation of this gene is useful in the treatment of liver related disorders.

Moderate levels of expression of this gene was also seen in cluster of cancer cell lines derived from pancreatic, gastric, colon, lung, liver, renal, breast, ovarian, prostate, squamous cell carcinoma, melanoma and brain cancers. Thus, expression of this gene is useful as a marker to detect these cancers. Therapeutic modulation of this gene, encoded protein and/or use of antibodies or small molecule targeting this gene or gene product is useful in the treatment of pancreatic, gastric, colon, lung, liver, renal, breast, ovarian, prostate, squamous cell carcinoma, melanoma and brain cancers.

Among tissues with metabolic or endocrine function, this gene was expressed at moderate levels in pancreas, adipose, adrenal gland, thyroid, pituitary gland, skeletal muscle, heart, liver and the gastrointestinal tract. Therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene, encoded protein and/or use of antibodies or small molecule targeting this gene or gene product is useful in the treatment of central nervous system disorders such as Alzheimer's disease, Parkinson's disease, epilepsy, multiple sclerosis, schizophrenia and depression.

Panel 1.3D Summary: Ag3086 This gene was highly expressed in both fetal and adult liver tissue (CTs=26) and liver cancer cell lines (CT=27). The gene was also expressed at moderate to low levels in most of the other tissues in the panel. Therapeutic modulation of this gene is useful in the treatment of liver related disorders.

In tissues involved in the central nervous system, this gene was moderately expressed in the fetal and adult brain, including the adult thalamus, substantia nigra, hippocampus, amygdala and is also expressed at low but significant levels in the cerebellum and cerebral cortex. This expression profile suggests that this gene has functional significance in the CNS. This gene codes for a homolog of hepatocyte growth factor, which has numerous therapeutic applications in the CNS, including prevention of neuronal death in animal models of stroke and ischemia. Hepatocyte growth factor has mitogenic activity and thus has potential application as a protein therapeutic to treat brain pathologies when administered directly to the cortico spinal fluid or systemically when the blood brain barrier is disrupted. Hepatocyte growth factor-like protein is a neurotrophic factor useful in the prevention of motoneuron atrophy upon axotomy. Therefore, this gene, expressed protein and/or use of antibodies or small molecule targeting this gene or gene product is useful as a therapeutic agent in treating stroke and neurodegenerative diseases including Alzheimer's disease, Parkinson's disease, and Huntington's disease. The potential role of the this gene or its protein product in brain plasticity and regeneration affords utility in treating brain damage and aging related disorders, such as memory impairment that has hippocampal dysfunction as its primary focus.

Panel 2.2 Summary: Ag3086 The expression of this gene was highest in a sample derived from a liver cancer specimen (CT=26) and was expressed at significant levels in a number of samples derived from liver tissue. There was significant expression of this gene associated with normal kidney tissue (CT=27.2) relative to adjacent kidney cancer specimens therapeutic modulation of this gene, encoded protein and/or use of antibodies or small molecule targeting this gene or gene product is useful in the treatment of kidney cancer.

Panel 4.1D Summary: Ag3797 Highest expression of this gene was detected in kidney (CTs=27.4-29). Moderate levels of expression of this gene was also seen in liver cirrhosis sample. This gene was expressed at moderate to low levels in a wide range of cell types of significance in the immune response in health and disease. These cells included members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression indicates that this gene is involved in homeostatic processes for these and other cell types and tissues. Therefore, modulation of this gene, encoded protein and/or use of antibodies or small molecule targeting this gene or gene product is useful in the treatment autoimmune and inflammatory diseases such as asthma, allergies, inflammatory bowel disease, lupus erythematosus, psoriasis, rheumatoid arthritis, and osteoarthritis.

AE. CG54479-06: Macrophage Stimulatory Protein.

Expression of gene CG54479-06 was assessed using the primer-probe set Ag6711, described in Table AEA. Results of the RTQ-PCR runs are shown in Table AEB.

TABLE AEA
Probe Name Ag6711

| | | | Start | SEQ ID |
|---|---|---|---|---|
| Primers | Sequences | Length | Position | No |
| Forward | 5′-accaagtgtgagggtgacta-3′ | 20 | 1861 | 1283 |
| Probe | TET-5′-tcctggaaggaattataatccccaacc- | 27 | 1919 | 1284 |
| | 3′-TAMRA | | | |
| Reverse | 5′-ccagtccacaaacacagaga-3′ | 20 | 1988 | 1285 |

TABLE AEB
General_screening_panel_v1.6
Column A - Rel. Exp.(%) Ag6711, Run 277261482

| Tissue Name | A | Tissue Name | A |
|---|---|---|---|
| Adipose | 3.5 | Renal ca. TK-10 | 60.7 |
| Melanoma* | 1.8 | Bladder | 11.4 |
| Hs688(A).T | | | |
| Melanoma* | 4.2 | Gastric ca. (liver met.) NCI-N87 | 5.6 |
| Hs688(B).T | | | |
| Melanoma* M14 | 1.1 | Gastric ca. KATO III | 1.4 |
| Melanoma* LOXIMVI | 0.7 | Colon ca. SW-948 | 0.9 |
| Melanoma* | 3.3 | Colon ca. SW480 | 12.7 |
| SK-MEL-5 | | | |
| Squamous cell | 0.5 | Colon ca.* (SW480 met) SW620 | 3.4 |
| carcinoma SCC-4 | | | |
| Testis Pool | 4.3 | Colon ca. HT29 | 0.3 |
| Prostate ca.* | 2.6 | Colon ca. HCT-116 | 0.5 |
| (bone met) PC-3 | | | |
| Prostate Pool | 1.1 | Colon ca. CaCo-2 | 28.7 |
| Placenta | 1.4 | Colon cancer tissue | 9.4 |
| Uterus Pool | 0.8 | Colon ca. SW1116 | 4.4 |
| Ovarian ca. OVCAR-3 | 5.1 | Colon ca. Colo-205 | 0.0 |
| Ovarian ca. SK-OV-3 | 1.0 | Colon ca. SW-48 | 1.0 |
| Ovarian ca. OVCAR-4 | 0.5 | Colon Pool | 3.0 |
| Ovarian ca. OVCAR-5 | 10.2 | Small Intestine Pool | 2.1 |
| Ovarian ca. IGROV-1 | 12.9 | Stomach Pool | 4.6 |
| Ovarian ca. OVCAR-8 | 8.3 | Bone Marrow Pool | 4.1 |
| Ovary | 3.8 | Fetal Heart | 1.1 |
| Breast ca. MCF-7 | 3.5 | Heart Pool | 1.2 |
| Breast ca. | 1.9 | Lymph Node Pool | 4.7 |
| MDA-MB-231 | | | |
| Breast ca. BT 549 | 2.9 | Fetal Skeletal Muscle | 2.5 |
| Breast ca. T47D | 0.0 | Skeletal Muscle Pool | 1.0 |
| Breast ca. MDA-N | 0.3 | Spleen Pool | 2.4 |
| Breast Pool | 6.0 | Thymus Pool | 7.9 |
| Trachea | 1.5 | CNS cancer (glio/astro) U87-MG | 4.8 |
| Lung | 12.9 | CNS cancer (glio/astro) U-118-MG | 4.6 |
| Fetal Lung | 3.5 | CNS cancer (neuro; met) SK-N-AS | 3.3 |
| Lung ca. NCI-N417 | 0.0 | CNS cancer (astro) SF-539 | 1.3 |
| Lung ca. LX-1 | 7.2 | CNS cancer (astro) SNB-75 | 1.7 |
| Lung ca. NCI-H146 | 1.7 | CNS cancer (glio) SNB-19 | 7.7 |
| Lung ca. SHP-77 | 6.3 | CNS cancer (glio) SF-295 | 8.1 |
| Lung ca. A549 | 1.8 | Brain (Amygdala) Pool | 1.2 |
| Lung ca. NCI-H526 | 0.0 | Brain (cerebellum) | 6.9 |
| Lung ca. NCI-H23 | 11.3 | Brain (fetal) | 4.1 |
| Lung ca. NCI-H460 | 5.9 | Brain (Hippocampus) Pool | 1.5 |
| Lung ca. HOP-62 | 3.8 | Cerebral Cortex Pool | 1.2 |
| Lung ca. NCI-H522 | 8.3 | Brain (Substantia nigra) Pool | 2.2 |
| Liver | 18.8 | Brain (Thalamus) Pool | 3.9 |
| Fetal Liver | 28.7 | Brain (whole) | 5.4 |
| Liver ca. HepG2 | 100.0 | Spinal Cord Pool | 4.4 |
| Kidney Pool | 7.3 | Adrenal Gland | 2.6 |
| Fetal Kidney | 7.7 | Pituitary gland Pool | 2.7 |
| Renal ca. 786-0 | 4.4 | Salivary Gland | 0.3 |
| Renal ca. A498 | 0.6 | Thyroid (female) | 4.0 |
| Renal ca. ACHN | 3.8 | Pancreatic ca. CAPAN2 | 1.4 |
| Renal ca. UO-31 | 8.8 | Pancreas Pool | 1.9 |

General_screening_panel_v1.6 Summary: Ag6711 Highest expression of this gene was detected in a liver cancer HepG2 cell line (CT=30.6). Significant expression of this gene was also seen in number of cancer cell lines derived from ovarian, lung, renal, colon and brain cancers. Thus, expression levels of this gene is useful as a marker to detect these cancers. Therapeutic modulation of this gene, encoded protein and/or use of antibodies or small molecule targeting this gene or gene product is useful in the treatment of pancreatic, gastric, colon, lung, liver, renal, breast, ovarian, prostate, squamous cell carcinoma, melanoma and brain cancers.

Moderate levels of expression of this gene was also seen in fetal and adult liver. Therapeutic modulation of this gene is useful in the treatment of liver related disorders such as obesity, liver cirrhosis and other liver inflammatory diseases.

AF. CG54539-02: Zinc Transporter 1.

Expression of gene CG54539-02 was assessed using the primer-probe set Ag1160, described in Table AFA. Results of the RTQ-PCR runs are shown in Tables AFB, AFC and AFD. CG54539-02 represents a full length physical clone.

TABLE AFA
Probe Name Ag1160

| | | | Start | SEQ ID |
|---|---|---|---|---|
| Primers | Sequences | Length | Position | No |
| Forward | 5′-atacatggaggtggctaaaacc-3′ | 22 | 1186 | 1286 |
| Probe | TET-5′-tcataatcacggaattcacgctactacca- | 29 | 1222 | 1287 |
| | 3′-TAMRA | | | |
| Reverse | 5′-acactagcaaattcaggctgaa-3′ | 22 | 1251 | 1288 |

TABLE AFB
General_screening_panel_v1.4
Column A - Rel. Exp.(%) Ag1160, Run 214691191

| Tissue Name | A | Tissue Name | A |
|---|---|---|---|
| Adipose | 46.0 | Renal ca. TK-10 | 35.4 |
| Melanoma* | 31.2 | Bladder | 35.4 |
| Hs688(A).T | | | |
| Melanoma* | 30.6 | Gastric ca. (liver met.) NCI-N87 | 32.1 |
| Hs688(B).T | | | |
| Melanoma* M14 | 23.0 | Gastric ca. KATO III | 31.2 |
| Melanoma* LOXIMVI | 14.1 | Colon ca. SW-948 | 8.8 |
| Melanoma* | 15.9 | Colon ca. SW480 | 17.6 |
| SK-MEL-5 | | | |
| Squamous Cell | 16.5 | Colon ca.* (SW480 met) SW620 | 18.0 |
| carcinoma SCC-4 | | | |
| Testis Pool | 62.4 | Colon ca. HT29 | 8.5 |
| Prostate ca.* | 13.9 | Colon ca. HCT-116 | 16.0 |
| (bone met) PC-3 | | | |
| Prostate Pool | 4.5 | Colon ca. CaCo-2 | 38.4 |
| Placenta | 5.3 | Colon cancer tissue | 18.6 |
| Uterus Pool | 5.1 | Colon ca. SW1116 | 4.7 |
| Ovarian ca. OVCAR-3 | 42.6 | Colon ca. Colo-205 | 11.5 |
| Ovarian ca. SK-OV-3 | 80.1 | Colon ca. SW-48 | 12.6 |
| Ovarian ca. OVCAR-4 | 8.5 | Colon Pool | 17.8 |
| Ovarian ca. OVCAR-5 | 36.9 | Small Intestine Pool | 9.3 |
| Ovarian ca. IGROV-1 | 15.4 | Stomach Pool | 9.7 |
| Ovarian ca. OVCAR-8 | 4.3 | Bone Marrow Pool | 6.8 |
| Ovary | 9.9 | Fetal Heart | 7.3 |
| Breast ca. MCF-7 | 37.4 | Heart Pool | 5.2 |
| Breast ca. | 32.1 | Lymph Node Pool | 17.0 |
| MDA-MB-231 | | | |
| Breast ca. BT 549 | 12.7 | Fetal Skeletal Muscle | 3.2 |
| Breast ca. T47D | 66.0 | Skeletal Muscle Pool | 1.8 |
| Breast ca. MDA-N | 15.3 | Spleen Pool | 100.0 |
| Breast Pool | 13.5 | Thymus Pool | 7.1 |
| Trachea | 8.5 | CNS cancer (glio/astro) U87-MG | 29.3 |
| Lung | 6.2 | CNS cancer (glio/astro) U-118-MG | 41.8 |
| Fetal Lung | 78.5 | CNS cancer (neuro; met) SK-N-AS | 28.5 |
| Lung ca. NCI-N417 | 4.7 | CNS cancer (astro) SF-539 | 7.0 |
| Lung ca. LX-1 | 29.1 | CNS cancer (astro) SNB-75 | 14.3 |
| Lung ca. NCI-H146 | 1.7 | CNS cancer (glio) SNB-19 | 15.1 |
| Lung ca. SHP-77 | 8.8 | CNS cancer (glio) SF-295 | 14.2 |
| Lung ca. A549 | 26.4 | Brain (Amygdala) Pool | 4.8 |
| Lung ca. NCI-H526 | 4.0 | Brain (cerebellum) | 5.1 |
| Lung ca. NCI-H23 | 12.8 | Brain (fetal) | 7.4 |
| Lung ca. NCI-H460 | 19.9 | Brain (Hippocampus) Pool | 7.3 |
| Lung ca. HOP-62 | 10.9 | Cerebral Cortex Pool | 13.0 |
| Lung ca. NCI-H522 | 3.8 | Brain (Substantia nigra) Pool | 7.1 |
| Liver | 3.4 | Brain (Thalamus) Pool | 9.6 |
| Fetal Liver | 70.2 | Brain (whole) | 9.1 |
| Liver ca. HepG2 | 30.4 | Spinal Cord Pool | 5.9 |
| Kidney Pool | 17.6 | Adrenal Gland | 24.7 |
| Fetal Kidney | 8.0 | Pituitary gland Pool | 2.7 |
| Renal ca. 786-0 | 20.9 | Salivary Gland | 3.7 |
| Renal ca. A498 | 20.7 | Thyroid (female) | 9.3 |
| Renal ca. ACHN | 29.9 | Pancreatic ca. CAPAN2 | 46.7 |
| Renal ca. UO-31 | 17.6 | Pancreas Pool | 17.4 |

TABLE AFC
Panel 2D
Column A - Rel. Exp.(%) Ag1160, Run 164339479

| Tissue Name | A | Tissue Name | A |
|---|---|---|---|
| Normal Colon | 63.3 | Kidney Margin 8120608 | 18.3 |
| CC Well to Mod Diff (ODO3866) | 17.0 | Kidney Cancer 8120613 | 3.9 |
| CC Margin (ODO3866) | 8.9 | Kidney Margin 8120614 | 10.6 |
| CC Gr.2 rectosigmoid (ODO3868) | 7.8 | Kidney Cancer 9010320 | 13.9 |
| CC Margin (ODO3868) | 2.0 | Kidney Margin 9010321 | 20.7 |
| CC Mod Diff (ODO3920) | 9.8 | Normal Uterus | 8.2 |
| CC Margin (ODO3920) | 15.2 | Uterine Cancer 064011 | 33.2 |
| CC Gr.2 ascend colon (ODO3921) | 28.9 | Normal Thyroid | 9.0 |
| CC Margin (ODO3921) | 6.4 | Thyroid Cancer | 52.9 |
| CC from Partial Hepatectomy | 51.8 | Thyroid Cancer A302152 | 20.7 |
| (ODO4309) Mets | | | |
| Liver Margin (ODO4309) | 65.5 | Thyroid Margin A302153 | 33.0 |
| Colon mets to lung (OD04451-01) | 8.4 | Normal Breast | 13.1 |
| Lung Margin (OD04451-02) | 13.5 | Breast Cancer | 19.5 |
| Normal Prostate 6546-1 | 26.8 | Breast Cancer (OD04590-01) | 29.7 |
| Prostate Cancer (OD04410) | 18.7 | Breast Cancer Mets (OD04590-03) | 27.0 |
| Prostate Margin (OD04410) | 15.1 | Breast Cancer Metastasis | 30.8 |
| Prostate Cancer (OD04720-01) | 17.2 | Breast Cancer | 16.2 |
| Prostate Margin (OD04720-02) | 18.3 | Breast Cancer | 15.0 |
| Normal Lung | 34.9 | Breast Cancer 9100266 | 48.3 |
| Lung Met to Muscle (ODO4286) | 39.0 | Breast Margin 9100265 | 15.5 |
| Muscle Margin (ODO4286) | 8.1 | Breast Cancer A209073 | 36.3 |
| Lung Malignant Cancer (OD03126) | 28.5 | Breast Margin A209073 | 11.5 |
| Lung Margin (OD03126) | 19.5 | Normal Liver | 39.0 |
| Lung Cancer (OD04404) | 20.0 | Liver Cancer | 14.6 |
| Lung Margin (OD04404) | 11.9 | Liver Cancer 1025 | 18.8 |
| Lung Cancer (OD04565) | 13.8 | Liver Cancer 1026 | 11.0 |
| Lung Margin (OD04565) | 3.9 | Liver Cancer 6004-T | 35.1 |
| Lung Cancer (OD04237-01) | 17.8 | Liver Tissue 6004-N | 13.1 |
| Lung Margin (OD04237-02) | 33.0 | Liver Cancer 6005-T | 12.7 |
| Ocular Mel Met to Liver (ODO4310) | 5.8 | Liver Tissue 6005-N | 8.0 |
| Liver Margin (ODO4310) | 100.0 | Normal Bladder | 33.4 |
| Melanoma Metastasis | 7.3 | Bladder Cancer | 7.0 |
| Lung Margin (OD04321) | 13.8 | Bladder Cancer | 11.5 |
| Normal Kidney | 37.4 | Bladder Cancer (OD04718-01) | 38.7 |
| Kidney Ca, Nuclear grade 2 (OD04338) | 46.0 | Bladder Normal Adjacent | 17.3 |
| | | (OD04718-03) | |
| Kidney Margin (OD04338) | 18.8 | Normal Ovary | 4.6 |
| Kidney Ca Nuclear grade ½ (OD04339) | 17.3 | Ovarian Cancer | 33.7 |
| Kidney Margin (OD04339) | 71.2 | Ovarian Cancer (OD04768-07) | 28.3 |
| Kidney Ca, Clear cell type (OD04340) | 69.3 | Ovary Margin (OD04768-08) | 5.7 |
| Kidney Margin (OD04340) | 23.3 | Normal Stomach | 19.5 |
| Kidney Ca, Nuclear grade 3 (OD04348) | 9.8 | Gastric Cancer 9060358 | 4.5 |
| Kidney Margin (OD04348) | 32.8 | Stomach Margin 9060359 | 18.6 |
| Kidney Cancer (OD04622-01) | 10.5 | Gastric Cancer 9060395 | 34.2 |
| Kidney Margin (OD04622-03) | 5.4 | Stomach Margin 9060394 | 13.9 |
| Kidney Cancer (OD04450-01) | 27.7 | Gastric Cancer 9060397 | 48.6 |
| Kidney Margin (OD04450-03) | 20.4 | Stomach Margin 9060396 | 3.4 |
| Kidney Cancer 8120607 | 6.2 | Gastric Cancer 064005 | 30.6 |

TABLE AFD
Panel 4D
Column A - Rel. Exp.(%) Ag1160, Run 140155352

| Tissue Name | A | Tissue Name | A |
|---|---|---|---|
| Secondary Th1 act | 14.0 | HUVEC IL-1beta | 9.7 |
| Secondary Th2 act | 24.0 | HUVEC IFN gamma | 15.5 |
| Secondary Tr1 act | 18.7 | HUVEC TNF alpha + IFN gamma | 1.9 |
| Secondary Th1 rest | 2.3 | HUVEC TNF alpha + IL4 | 7.0 |
| Secondary Th2 rest | 4.3 | HUVEC IL-11 | 5.8 |
| Secondary Tr1 rest | 2.8 | Lung Microvascular EC none | 16.6 |
| Primary Th1 act | 21.3 | Lung Microvascular EC TNFalpha + IL- | 7.1 |
| | | 1beta | |
| Primary Th2 act | 20.6 | Microvascular Dermal EC none | 58.6 |
| Primary Tr1 act | 31.4 | Microsvasular Dermal EC TNFalpha + IL- | 16.8 |
| | | 1beta | |
| Primary Th1 rest | 16.4 | Bronchial epithelium TNFalpha + IL1beta | 40.3 |
| Primary Th2 rest | 10.3 | Small airway epithelium none | 14.6 |
| Primary Tr1 rest | 14.2 | Small airway epithelium TNFalpha + IL- | 92.0 |
| | | 1beta | |
| CD45RA CD4 lymphocyte act | 13.3 | Coronery artery SMC rest | 22.1 |
| CD45RO CD4 lymphocyte act | 11.7 | Coronery artery SMC TNFalpha + IL-1beta | 7.5 |
| CD8 lymphocyte act | 6.8 | Astrocytes rest | 8.0 |
| Secondary CD8 lymphocyte | 5.9 | Astrocytes TNFalpha + IL-1beta | 16.7 |
| rest | | | |
| Secondary CD8 lymphocyte act | 10.5 | KU-812 (Basophil) rest | 18.3 |
| CD4 lymphocyte none | 11.4 | KU-812 (Basophil) PMA/ionomycm | 69.3 |
| 2ry Th1/Th2/Tr1 anti-CD95 | 11.5 | CCD1106 (Keratinocytes) none | 13.7 |
| CH11 | | | |
| LAK cells rest | 66.0 | 93580 CCD1106 (Keratinocytes) TNFa and | 56.6 |
| | | IFNg | |
| LAK cells IL-2 | 13.8 | Liver cirrhosis | 7.6 |
| LAK cells IL-2 + IL-12 | 24.0 | Lupus kidney | 4.5 |
| LAK cells IL-2 + IFN gamma | 24.5 | NCI-H292 none | 37.9 |
| LAK cells IL-2 + IL-18 | 20.7 | NCI-H292 IL-4 | 51.8 |
| LAK cells PMA/ionomycin | 14.1 | NCI-H292 IL-9 | 48.0 |
| NK Cells IL-2 rest | 5.9 | NCI-H292 IL-13 | 26.2 |
| Two Way MLR 3 day | 35.6 | NCI-H292 IFN gamma | 27.9 |
| Two Way MLR 5 day | 23.2 | HPAEC none | 14.0 |
| Two Way MLR 7 day | 7.7 | HPAEC TNF alpha + IL-1 beta | 7.3 |
| PBMC rest | 7.4 | Lung fibroblast none | 14.7 |
| PBMC PWM | 100.0 | Lung fibroblast TNF alpha + IL-1 beta | 9.9 |
| PBMC PHA-L | 31.6 | Lung fibroblast IL-4 | 36.1 |
| Ramos (B cell) none | 29.5 | Lung fibroblast IL-9 | 27.9 |
| Ramos (B cell) ionomycin | 47.0 | Lung fibroblast IL-13 | 65.1 |
| B lymphocytes PWM | 36.6 | Lung fibroblast IFN gamma | 66.9 |
| B lymphocytes CD40L and IL- | 8.5 | Dermal fibroblast CCD1070 rest | 38.2 |
| 4 | | | |
| EOL-1 dbcAMP | 4.7 | Dermal fibroblast CCD1070 TNF alpha | 50.3 |
| EOL-1 dbcAMP | 15.8 | Dermal fibroblast CCD1070 IL-1 beta | 27.2 |
| PMA/ionomycin | | | |
| Dendritic cells none | 30.1 | Dermal fibroblast IFN gamma | 7.6 |
| Dendritic cells LPS | 34.4 | Dermal fibroblast IL-4 | 16.0 |
| Dendritic cells anti-CD40 | 39.5 | IBD Colitis 2 | 1.1 |
| Monocytes rest | 26.1 | IBD Crohn's | 3.5 |
| Monocytes LPS | 88.9 | Colon | 38.7 |
| Macrophages rest | 96.6 | Lung | 24.1 |
| Macrophages LPS | 68.8 | Thymus | 94.0 |
| HUVEC none | 15.9 | Kidney | 10.2 |
| HUVEC starved | 26.6 | | |

General_screening_panel_v1.4 Summary: Ag1160 Highest expression was detected in spleen (CT=27). This gene was widely expressed in this panel, demonstrating a role for this gene product in cell survival and proliferation. Moderate expression was also seen in brain, colon, gastric, lung, breast, ovarian, and melanoma cancer cell lines. Modulation of this gene product is useful in the treatment of these cancers.

Among tissues with metabolic function, this gene was expressed at moderte to low levels in pituitary, adipose, adrenal gland, pancreas, thyroid, and adult and fetal skeletal muscle, heart, and liver. This widespread expression among these tissues shows that this gene product plays a role in normal neuroendocrine and metabolic function and that disregulated expression of this gene contributes to neuroendocrine disorders or metabolic diseases, such as obesity and diabetes.

This gene was expressed at much higher levels in fetal lung and liver tissue (CTs=27) when compared to expression in the adult counterparts (CTs=30-31). Expression of this gene is useful as a marker to differentiate between the fetal and adult sources of these tissues. The relative overexpression of this gene in fetal lung shows that the protein product enhances lung and liver growth or development in the fetus and also acts in a regenerative capacity in the adult. Therapeutic modulation of the protein encoded by this gene is useful in treatment of lung and liver related diseases.

This gene was also expressed at moderate to low in the CNS, including the hippocampus, thalamus, substantia nigra, amygdala, cerebellum and cerebral cortex. Therapeutic modulation of the expression or function of this gene is useful in the treatment of neurologic disorders, such as Alzheimer's disease, Parkinson's disease, schizophrenia, multiple sclerosis, stroke and epilepsy.

Panel 2D Summary: Ag1160 Highest expression of this gene was detected in normal liver (CT=27). This gene was over expressed in gastric, colon, breast and lung cancers samples compared to the normal adjacent tissues. Targeting this gene or its protein product with a small molecule, antibody or protein therapeutic is useful in the treatment of these cancers.

Panel 4D Summary: Ag1160 This gene was upregulated in several normal and activated tissues. This gene was particularly high in activated monocytes and both activated and resting macrophages. Antibodies targeting this gene or gene product is useful to detect monocytes which are differentiating into macrophages. Antagonistic therapeutics to this molecule will inhibit the differentiation process, activation of the epithelium or keratinocytes in the skin and block or reduce inflammation in diseases such as asthma, allergy, psoriasis and emphysema.

AG. CG54683-05: Gamma-Aminobutyric-Acid Receptor RHO-3 Subunit Precursor.

Expression of gene CG54683-05 was assessed using the primer-probe sets Ag1130, Ag1198, Ag1253, Ag1603 and Ag3363, described in Tables AGA, AGB, AGC, AGD and AGE. Results of the RTQ-PCR runs are shown in Tables AGF and AGG.

TABLE AGA
Probe Name Ag1130

| | | | | |
|---|---|---|---|---|
| | | | Start | SEQ ID |
| Primers | Sequences | Length | Position | No |

| | | | | |
|---|---|---|---|---|
| Forward | 5′-gtcctggctttccagttagtct-3′ | 22 | 7 | 1289 |
| Probe | TET-5′-tcacctacatctggatcatattgaaacca- | 29 | 32 | 1290 |
| | 3′-TAMRA | | | |
| Reverse | 5′-ttgatgttagaagcagcacaaa-3′ | 22 | 65 | 1291 |

TABLE AGB
Probe Name Ag1198

| | | | | |
|---|---|---|---|---|
| | | | Start | SEQ ID |
| Primers | Sequences | Length | Position | No |

| | | | | |
|---|---|---|---|---|
| Forward | 5′-gtcctggctttccagttagtct-3′ | 22 | 7 | 1292 |
| Probe | TET-5′-tcacctacatctggatcatattgaaacca- | 29 | 32 | 1293 |
| | 3′-TAMRA | | | |
| Reverse | 5′-ttgatgttagaagcagcacaaa-3′ | 22 | 65 | 1294 |

TABLE AGC
Probe Name Ag1253

| | | | Start | SEQ ID |
|---|---|---|---|---|
| Primers | Sequences | Length | Position | No |
| Forward | 5′-atctgggtgcctgatatctttt-3′ | 22 | 415 | 1295 |
| Probe | TET-5′-tgtccactctaaaagatccttcatccat | 30 | 438 | 1296 |
| | ga-3′-TAMRA | | | |
| Reverse | 5′-cgcagcatgatattctccatag-3′ | 22 | 473 | 1297 |

TABLE AGD
Probe Name Ag1603

| | | | | |
|---|---|---|---|---|
| | | | Start | SEQ ID |
| Primers | Sequences | Length | Position | No |

| | | | | |
|---|---|---|---|---|
| Forward | 5′-gtcctggctttccagttagtct-3′ | 22 | 7 | 1298 |
| Probe | TET-5′-tcacctacatctggatcatattgaaacca- | 29 | 32 | 1299 |
| | 3′-TAMRA | | | |
| Reverse | 5′-ttgatgttagaagcagcacaaa-3′ | 22 | 65 | 1300 |

TABLE AGE
Probe Name Ag3363

| | | | Start | SEQ ID |
|---|---|---|---|---|
| Primers | Sequences | Length | Position | No |
| Forward | 5′-tggctttccagttagtctcctt-3′ | 22 | 11 | 1301 |
| Probe | TET-5′-cacctacatctggatcatattgaaacca- | 28 | 33 | 1302 |
| | 3′-TAMRA | | | |
| Reverse | 5′-ttgatgttagaagcagcacaaa-3′ | 22 | 65 | 1303 |

TABLE AGF
Panel 1.2
Column A - Rel. Exp.(%) Ag1130, Run 125117140
Column B - Rel. Exp.(%) Ag1130, Run 126566764
Column C - Rel. Exp.(%) Ag1198, Run 129140506

| Tissue Name | A | B | C | Tissue Name | A | B | C |
|---|---|---|---|---|---|---|---|
| Endothelial cells | 0.0 | 0.0 | 0.0 | Renal ca. 786-0 | 0.0 | 0.0 | 0.0 |
| Heart (Fetal) | 0.0 | 0.0 | 0.0 | Renal ca. A498 | 7.3 | 4.7 | 0.0 |
| Pancreas | 0.0 | 0.0 | 0.0 | Renal ca. RXF 393 | 0.0 | 0.0 | 0.0 |
| Pancreatic ca. CAPAN 2 | 9.0 | 0.0 | 0.0 | Renal ca. ACHN | 0.0 | 0.0 | 0.0 |
| Adrenal gland | 0.0 | 2.6 | 0.0 | Renal ca. UO-31 | 3.9 | 0.0 | 0.0 |
| Thyroid | 0.0 | 0.0 | 0.0 | Renal ca. TK-10 | 0.0 | 0.0 | 0.0 |
| Salivary gland | 0.0 | 0.0 | 0.0 | Liver | 26.6 | 0.0 | 0.0 |
| Pituitary gland | 0.0 | 0.0 | 0.0 | Liver (fetal) | 25.3 | 0.0 | 0.0 |
| Brain (fetal) | 0.0 | 0.0 | 0.0 | Liver ca. (hepatoblast) | 0.0 | 0.0 | 0.0 |
| | | | | HepG2 | | | |
| Brain (whole) | 2.6 | 20.0 | 0.0 | Lung | 0.0 | 0.0 | 0.0 |
| Brain (amygdala) | 1.3 | 32.1 | 0.0 | Lung (fetal) | 0.0 | 0.0 | 0.0 |
| Brain (cerebellum) | 1.5 | 3.8 | 0.0 | Lung ca. (small cell) LX-1 | 3.4 | 0.0 | 0.0 |
| Brain (hippocampus) | 0.0 | 27.0 | 0.0 | Lung ca. (small cell) NCI- | 28.5 | 74.2 | 0.0 |
| | | | | H69 | | | |
| Brain (thalamus) | 9.9 | 22.5 | 9.8 | Lung ca. (s.cell var.) SHP- | 3.8 | 9.7 | 0.0 |
| | | | | 77 | | | |
| Cerebral Cortex | 0.0 | 0.0 | 0.0 | Lung ca. (large cell) NCI- | 8.8 | 4.1 | 5.3 |
| | | | | H460 | | | |
| Spinal cord | 4.4 | 0.0 | 0.0 | Lung ca. (non-sm. cell) | 51.4 | 9.5 | 7.2 |
| | | | | A549 | | | |
| glio/astro U87-MG | 0.0 | 0.0 | 0.0 | Lung ca. (non-s.cell) Nd- | 0.0 | 0.0 | 0.0 |
| | | | | H23 | | | |
| glio/astro U-118-MG | 0.0 | 0.0 | 0.0 | Lung ca. (non-s.cell) | 8.4 | 2.7 | 9.6 |
| | | | | HOP-62 | | | |
| astrocytoma SW1783 | 2.9 | 0.0 | 0.0 | Lung ca. (non-s.cl) NCI- | 0.0 | 0.0 | 0.0 |
| | | | | H522 | | | |
| neuro*; met SK-N-AS | 0.0 | 0.0 | 0.0 | Lung ca. (squam.) SW | 3.2 | 8.7 | 0.0 |
| | | | | 900 | | | |
| astrocytoma SF-539 | 5.1 | 0.0 | 0.0 | Lung ca. (squam.) NCI | 2.3 | 15.9 | 0.0 |
| astrocytoma SNB-75 | 2.3 | 0.0 | 0.0 | Mammary gland | 0.0 | 0.0 | 0.0 |
| glioma SNB-19 | 6.3 | 20.7 | 9.0 | Breast ca.* (pl.ef) MCF-7 | 0.0 | 0.0 | 0.0 |
| glioma U251 | 1.4 | 0.0 | 1.8 | Breast ca.* (pl.ef) MDA- | 0.0 | 0.0 | 0.0 |
| | | | | MB-231 | | | |
| glioma SF-295 | 0.0 | 0.0 | 0.0 | Breast ca.* (pl.ef) T47D | 14.1 | 37.4 | 0.0 |
| Heart | 0.0 | 0.0 | 0.0 | Breast ca. BT-549 | 12.5 | 21.0 | 12.3 |
| Skeletal muscle | 2.3 | 0.0 | 0.0 | Breast ca. MDA-N | 0.0 | 0.0 | 0.0 |
| Bone marrow | 0.0 | 0.0 | 0.0 | Ovary | 0.0 | 0.0 | 0.0 |
| Thymus | 0.0 | 0.0 | 0.0 | Ovarian ca. OVCAR-3 | 0.0 | 0.0 | 0.0 |
| Spleen | 2.2 | 0.0 | 0.0 | Ovarian ca. OVCAR-4 | 0.0 | 0.0 | 0.0 |
| Lymph node | 0.0 | 0.0 | 0.0 | Ovarian ca. OVCAR-5 | 66.9 | 35.4 | 4.4 |
| Colorectal | 11.3 | 27.7 | 21.8 | Ovarian ca. OVCAR-8 | 2.7 | 0.0 | 0.0 |
| Stomach | 0.0 | 0.0 | 0.0 | Ovarian ca. IGROV-1 | 6.0 | 0.0 | 0.0 |
| Small intestine | 5.4 | 0.0 | 0.0 | Ovarian ca. (ascites) SK- | 30.8 | 0.0 | 0.0 |
| | | | | OV-3 | | | |
| Colon ca. SW480 | 3.2 | 0.0 | 0.0 | Uterus | 0.0 | 0.0 | 0.0 |
| Colon ca.* SW620 | 0.0 | 0.0 | 0.0 | Placenta | 0.0 | 0.0 | 0.0 |
| (SW480 met) | | | | | | | |
| Colon ca. HT29 | 1.9 | 14.4 | 0.0 | Prostate | 6.9 | 0.0 | 0.0 |
| Colon ca. HCT-116 | 0.0 | 0.0 | 0.0 | Prostate ca.* (bone met) | 100.0 | 0.0 | 0.0 |
| | | | | PC-3 | | | |
| Colon ca. CaCo-2 | 0.0 | 0.0 | 0.0 | Testis | 54.7 | 100.0 | 36.9 |
| CC Well to Mod Duff | 72.2 | 75.8 | 100.0 | Melanoma Hs688(A).T | 4.2 | 0.0 | 0.0 |
| (ODO3866) | | | | | | | |
| Colon ca. HCC-2998 | 5.3 | 4.8 | 0.0 | Melanoma* (met) | 2.7 | 34.2 | 13.3 |
| | | | | Hs688(B).T | | | |
| Gastric ca. (liver met) NCI- | 50.3 | 0.0 | 0.0 | Melanoma UACC-62 | 0.0 | 0.0 | 0.0 |
| N87 | | | | | | | |
| Bladder | 6.0 | 22.1 | 0.0 | Melanoma M14 | 31.4 | 36.3 | 20.2 |
| Trachea | 0.0 | 0.0 | 0.0 | Melanoma LOX IMVI | 0.0 | 0.0 | 0.0 |
| Kidney | 2.0 | 0.0 | 0.0 | Melanoma* (met) SK- | 2.4 | 0.0 | 0.0 |
| | | | | MEL-5 | | | |
| Kidney (fetal) | 1.1 | 2.5 | 0.0 | | | | |

TABLE AGG
Panel 4R
Column A - Rel. Exp.(%) Ag1198, Run 142014937

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 2.5 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 0.0 |
| Primary Tn act | 0.0 | Microsvasular Dermal EC TNFalpha + IL- | 0.0 |
| | | 1beta | |
| Primary Th1 rest | 0.0 | Bronchial epithelium TNFalpha + IL1beta | 0.0 |
| Primary Th2 rest | 0.0 | Small airway epithelium none | 0.0 |
| Primary Tr1 rest | 0.0 | Small airway epithelium TNFalpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL-Ibeta | 0.0 |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | 93580 CCD1106 (Keratinocytes) TNFa and | 0.0 |
| | | IFNg | |
| LAK cells IL-2 | 0.0 | Liver cirrhosis | 16.4 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 beta | 0.0 |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycm | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF alpha | 0.0 |
| EOL-1 dbcAMP | | | |
| PMA/ionomycin | 0.0 | Dermal fibroblast CCD1070 IL-1 beta | 0.0 |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 1 | 100.0 |
| Monocytes rest | 10.0 | IBD Colitis 2 | 0.0 |
| Monocytes LPS | 0.0 | IBD Crohn's | 0.0 |
| Macrophages rest | 0.0 | Colon | 0.0 |
| Macrophages LPS | 0.0 | Lung | 0.0 |
| HUVEC none | 0.0 | Thymus | 0.0 |
| HUVEC starved | 0.0 | Kidney | 0.0 |

Panel 1.2 Summary: Ag1130/Ag1198—Significant expression of this gene was seen in testis, a colon cancer and prostate cancer cell line (CTs=33-34). Therefore, modulation of this gene is useful in the treatment of colon and prostate cancers.

Panel 4R Summary: Ag1198 Significant expression of this gene was seen only in the IBD colitis 1 sample (CT=34.2). Modulation of this gene is useful in the treatment of IBD colitis.

AH. CG54692-06: 5-Hydroxytryptamine 5A Receptor.

Expression of gene CG54692-06 was assessed using the primer-probe sets Ag1507, Ag1558 and Ag1602, described in Tables AHA, AHB and AHC. Results of the RTQ-PCR runs are shown in Tables AHD and AHE.

TABLE AHA
Probe Name Ag1507

| | | | Start | SEQ ID |
|---|---|---|---|---|
| Primers | Sequences | Length | Position | No |
| Forward | 5′-cccctgatttacacagctttta-3′ | 22 | 1042 | 1304 |
| Probe | TET-5′-acaacaatgccttcaagagcctcttt-3′- | 26 | 1073 | 1305 |
| | TAMRA | | | |
| Reverse | 5′-ccctgtgttcatctctgcttag-3′ | 22 | 1100 | 1306 |

TABLE AHB
Probe Name Ag1558

| | | | Start | SEQ ID |
|---|---|---|---|---|
| Primers | Sequences | Length | Position | No |
| Forward | 5′-cccctgatttacacagctttta-3′ | 22 | 1042 | 1307 |
| Probe | TET-5′-acaacaatgccttcaagagcctcttt-3′- | 26 | 1073 | 1308 |
| | TAMRA | | | |
| Reverse | 5′-ccctgtgttcatctctgcttag-3′ | 22 | 1100 | 1309 |

TABLE AHC
Probe Name Ag1602

| | | | Start | SEQ ID |
|---|---|---|---|---|
| Primers | Sequences | Length | Position | No |
| Forward | 5′-cccctgatttacacagctttta-3′ | 22 | 1042 | 1310 |
| Probe | TET-5′-acaacaatgccttcaagagcctcttt-3′- | 26 | 1073 | 1311 |
| | TAMRA | | | |
| Reverse | 5′-ccctgtgttcatctctgcttag-3′ | 22 | 1100 | 1312 |

TABLE AHD
Panel 1.2
Column A - Rel. Exp.(%) Ag1507, Run 142131135

| Tissue Name | A | Tissue Name | A |
|---|---|---|---|
| Endothelial cells | 7.5 | Renal ca. 786-0 | 0.9 |
| Heart (Fetal) | 5.3 | Renal ca. A498 | 27.4 |
| Pancreas | 8.7 | Renal ca. RXF 393 | 2.3 |
| Pancreatic ca. CAPAN 2 | 3.0 | Renal ca. ACHN | 15.5 |
| Adrenal Gland | 4.4 | Renal ca. UO-31 | 19.2 |
| Thyroid | 2.3 | Renal ca. TK-10 | 39.8 |
| Salivary gland | 12.9 | Liver | 5.8 |
| Pituitary gland | 0.0 | Liver (fetal) | 0.0 |
| Brain (fetal) | 0.0 | Liver ca. (hepatoblast) HepG2 | 27.2 |
| Brain (whole) | 4.0 | Lung | 0.0 |
| Brain (amygdala) | 20.3 | Lung (fetal) | 1.0 |
| Brain (cerebellum) | 3.2 | Lung ca. (small cell) LX-1 | 9.0 |
| Brain (hippocampus) | 13.0 | Lung ca. (small cell) NCI-H69 | 34.6 |
| Brain (thalamus) | 3.6 | Lung ca. (s.cell var.) SHP-77 | 2.0 |
| Cerebral Cortex | 16.6 | Lung ca. (large cell) NCI-H460 | 4.9 |
| Spinal cord | 0.0 | Lung ca. (non-sm. cell) A549 | 19.6 |
| gilo/astro U87-MG | 10.6 | Lung ca. (non-s.cell) NCI-H23 | 25.7 |
| glio/astro U-118-MG | 3.8 | Lung ca. (non-s.cell) HOP-62 | 35.8 |
| astrocytoma SW1783 | 2.0 | Lung ca. (non-s.cl) NCI-H522 | 21.6 |
| neuro*; met SK-N-AS | 1.6 | Lung ca. (squam.) SW 900 | 21.2 |
| astrocytoma SF-539 | 4.7 | Lung ca. (squam.) NCI-H596 | 3.3 |
| astrocytoma SNB-75 | 2.1 | Mammary gland | 1.1 |
| glioma SNB-19 | 16.4 | Breast ca.* (pl.ef) MCF-7 | 2.0 |
| glioma U251 | 9.2 | Breast ca.* (pl.ef) MDA-MB-231 | 2.9 |
| glioma SF-295 | 3.2 | Breast ca.* (pl.ef) T47D | 20.7 |
| Heart | 19.2 | Breast ca. BT-549 | 11.4 |
| Skeletal Muscle | 1.4 | Breast ca. MDA-N | 30.1 |
| Bone marrow | 0.9 | Ovary | 17.0 |
| Thymus | 0.0 | Ovarian ca. OVCAR-3 | 5.3 |
| Spleen | 3.9 | Ovarian ca. OVCAR-4 | 13.9 |
| Lymph node | 1.2 | Ovarian ca. OVCAR-5 | 100.0 |
| Colorectal | 6.0 | Ovarian ca. OVCAR-8 | 72.7 |
| Stomach | 0.9 | Ovarian ca. IGROV-1 | 49.3 |
| Small intestine | 6.0 | Ovarian ca. (ascites) SK-OV-3 | 36.1 |
| Colon ca. SW480 | 2.3 | Uterus | 1.0 |
| Colon ca.* SW620 (SW480 met) | 0.0 | Placenta | 0.0 |
| Colon ca. HT29 | 14.6 | Prostate | 3.0 |
| Colon ca. HCT-116 | 13.5 | Prostate ca.* (bone met) PC-3 | 16.0 |
| Colon ca. CaCo-2 | 3.5 | Testis | 30.8 |
| CC Well to Mod Diff (ODO3866) | 17.8 | Melanoma Hs688(B).T | 2.0 |
| Colon ca. HCC-2998 | 35.1 | Melanoma* (met) Hs688(B).T | 7.3 |
| Gastric ca. (liver met) NCI-N87 | 14.6 | Melanoma UACC-62 | 6.0 |
| Bladder | 38.4 | Melanoma M14 | 57.4 |
| Trachea | 0.0 | Melanoma LOX IMVI | 12.2 |
| Kidney | 28.5 | Melanoma* (met) SK-MEL-5 | 3.7 |
| Kidney (fetal) | 8.4 | | |

TABLE AHE
Panel 2D
Column A - Rel. Exp. (%) Ag1507, Run 165723610
Column B - Rel. Exp. (%) Ag1558, Run 157982934
Column C - Rel. Exp. (%) Ag1602, Run 162381799

| Tissue Name | A | B | C | Tissue Name | A | B | C |
|---|---|---|---|---|---|---|---|
| Normal Colon | 11.9 | 23.8 | 35.6 | Kidney Margin 8120608 | 0.0 | 0.0 | 0.0 |
| CC Well to Mod Diff | 47.0 | 19.5 | 47.3 | Kidney Cancer 8120613 | 0.0 | 0.0 | 3.8 |
| (ODO3866) | | | | | | | |
| CC Margin (ODO3866) | 41.5 | 9.9 | 11.3 | Kidney Margin 8120614 | 0.0 | 0.0 | 0.0 |
| CC Gr.2 rectosigmoid | 24.0 | 8.2 | 27.2 | Kidney Cancer 9010320 | 0.0 | 0.0 | 14.2 |
| (ODO3868) | | | | | | | |
| CC Margin (ODO3868) | 16.5 | 0.0 | 4.0 | Kidney Margin 9010321 | 11.9 | 0.0 | 18.3 |
| CC Mod Diff (ODO3920) | 62.0 | 17.6 | 0.0 | Normal Uterus | 0.0 | 13.1 | 0.0 |
| CC Margin (ODO3920) | 11.2 | 16.4 | 9.0 | Uterine Cancer 064011 | 0.0 | 8.9 | 18.2 |
| CC Gr.2 ascend colon | 6.9 | 28.9 | 0.0 | Normal Thyroid | 14.0 | 0.0 | 0.0 |
| (ODO3921) | | | | | | | |
| CC Margin (ODO3921) | 49.3 | 17.9 | 27.9 | Thyroid Cancer | 0.0 | 0.0 | 0.0 |
| CC from Partial | 12.5 | 9.3 | 8.1 | Thyroid Cancer | 0.0 | 0.0 | 5.0 |
| Hepatectomy (ODO4309) | | | | A302152 | | | |
| Mets | | | | | | | |
| Liver Margin (ODO4309) | 0.0 | 0.0 | 8.7 | Thyroid Margin | 19.2 | 30.4 | 18.7 |
| | | | | A302153 | | | |
| Colon mets to lung | 13.7 | 0.0 | 9.0 | Normal Breast | 0.0 | 21.9 | 0.0 |
| (OD04451-01) | | | | | | | |
| Lung Margin (OD04451-02) | 12.2 | 0.0 | 15.5 | Breast Cancer | 0.0 | 8.1 | 31.0 |
| Normal Prostate 6546-1 | 10.5 | 0.0 | 22.7 | Breast Cancer | 0.0 | 24.7 | 7.7 |
| | | | | (OD04590-01) | | | |
| Prostate Cancer (OD04410) | 0.0 | 8.4 | 0.0 | Breast Cancer Mets | 10.8 | 20.3 | 10.9 |
| | | | | (OD04590-03) | | | |
| Prostate Margin (OD04410) | 0.0 | 8.0 | 10.8 | Breast Cancer | 0.0 | 11.7 | 40.9 |
| | | | | Metastasis | | | |
| Prostate Cancer (OD04720- | 9.8 | 9.3 | 25.9 | Breast Cancer | 34.9 | 25.2 | 8.5 |
| 01) | | | | | | | |
| Prostate Margin (OD04720- | 39.0 | 33.7 | 25.7 | Breast Cancer | 30.6 | 8.5 | 0.0 |
| 02) | | | | | | | |
| Normal Lung | 24.5 | 60.3 | 100.0 | Breast Cancer 9100266 | 19.9 | 0.0 | 0.0 |
| Lung Met to Muscle | 7.0 | 7.1 | 27.2 | Breast Margin 9100265 | 0.0 | 7.4 | 0.0 |
| (ODO4286) | | | | | | | |
| Muscle Margin (ODO4286) | 0.0 | 0.0 | 28.5 | Breast Cancer A209073 | 44.8 | 25.3 | 9.0 |
| Lung Malignant Cancer | 0.0 | 9.8 | 11.5 | Breast Margin A209073 | 11.6 | 8.8 | 25.9 |
| (OD03126) | | | | | | | |
| Lung Margin (OD03126) | 20.0 | 35.6 | 11.2 | Normal Liver | 40.6 | 19.6 | 17.3 |
| Lung Cancer (OD04404) | 9.5 | 4.7 | 10.1 | Liver Cancer | 0.0 | 16.6 | 13.6 |
| Lung Margin (OD04404) | 0.0 | 8.9 | 0.0 | Liver Cancer 1025 | 0.0 | 8.6 | 10.2 |
| Lung Cancer (OD04565) | 0.0 | 0.0 | 0.0 | Liver Cancer 1026 | 0.0 | 9.5 | 0.0 |
| Lung Margin (OD04565) | 10.4 | 11.3 | 7.4 | Liver Cancer 6004-T | 0.0 | 24.7 | 9.3 |
| Lung Cancer (OD04237-01) | 10.4 | 8.1 | 0.0 | Liver Tissue 6004-N | 0.0 | 9.3 | 0.0 |
| Lung Margin (OD04237-02) | 18.7 | 7.6 | 17.7 | Liver Cancer 6005-T | 0.0 | 0.0 | 10.0 |
| Ocular Mel Met to Liver | 0.0 | 0.0 | 0.0 | Liver Tissue 6005-N | 0.0 | 0.0 | 0.0 |
| (ODO4310) | | | | | | | |
| Liver Margin (ODO4310) | 0.0 | 0.0 | 0.0 | Normal Bladder | 10.4 | 9.9 | 0.0 |
| Melanoma Metastasis | 0.0 | 6.7 | 0.0 | Bladder Cancer | 0.0 | 9.0 | 0.0 |
| Lung Margin (OD04321) | 0.0 | 0.0 | 27.4 | Bladder Cancer | 54.7 | 54.7 | 32.1 |
| Normal Kidney | 11.3 | 36.1 | 9.5 | Bladder Cancer | 0.0 | 8.0 | 9.3 |
| | | | | (OD04718-0l) | | | |
| Kidney Ca, Nuclear grade 2 | 0.0 | 0.0 | 0.0 | Bladder Normal | 0.0 | 17.2 | 6.3 |
| (OD04338) | | | | Adjacent (OD04718-03) | | | |
| Kidney Margin (OD04338) | 14.4 | 0.0 | 0.0 | Normal Ovary | 0.0 | 0.0 | 8.5 |
| Kidney Ca Nuclear grade 1/2 | 0.0 | 15.9 | 27.5 | Ovarian Cancer | 11.1 | 17.6 | 10.2 |
| (OD04339) | | | | | | | |
| Kidney Margin (OD04339) | 21.0 | 8.6 | 28.5 | Ovarian Cancer | 0.0 | 9.4 | 27.0 |
| | | | | (OD04768-07) | | | |
| Kidney Ca, Clear cell type | 0.0 | 14.0 | 16.0 | Ovary Margin | 0.0 | 0.0 | 0.0 |
| (OD04340) | | | | (OD04768-08) | | | |
| Kidney Margin (OD04340) | 18.6 | 0.0 | 17.9 | Normal Stomach | 24.5 | 7.7 | 5.0 |
| Kidney Ca, Nuclear grade 3 | 0.0 | 16.8 | 0.0 | Gastric Cancer 9060358 | 0.0 | 0.0 | 0.0 |
| (OD04348) | | | | | | | |
| Kidney Margin (OD04348) | 6.3 | 29.3 | 9.0 | Stomach Margin | 0.0 | 8.5 | 0.0 |
| | | | | 9060359 | | | |
| Kidney Cancer (OD04622- | 0.0 | 0.0 | 0.0 | Gastric Cancer 9060395 | 57.4 | 4.9 | 3.9 |
| 01) | | | | | | | |
| Kidney Margin (OD04622- | 0.0 | 0.0 | 0.0 | Stomach Margin | 0.0 | 32.8 | 18.2 |
| 03) | | | | 9060394 | | | |
| Kidney Cancer (OD04450- | 0.0 | 0.0 | 14.0 | Gastric Cancer 9060397 | 24.1 | 10.2 | 9.9 |
| 01) | | | | | | | |
| Kidney Margin (OD04450- | 0.0 | 5.5 | 0.0 | Stomach Margin | 15.1 | 0.0 | 0.0 |
| 03) | | | | 9060396 | | | |
| Kidney Cancer 8120607 | 0.0 | 0.0 | 0.0 | Gastric Cancer 064005 | 100.0 | 100.0 | 50.7 |

Panel 1.2 Summary: Ag1507 Low but significant expression of this gene was detected in ovarian cancer cell lines (CT=32.5). In general, expression of this gene was seen in cancer cell lines rather than in normal tissues, with low but significant expression also detectable in melanoma, breast cancer, lung cancer, and renal cancer cell lines. Thus, expression levels of this gene is useful to detect melanoma, breast, lung, renal and colon cancers. Therapeutic inhibition of the this gene or gene product, and/or use of antibodies, small molecule or protein drugs, is effective in the treatment of the afore mentioned cancers.

Panel 2D Summary: Ag1558 Significant expression of this gene was detected in a gastric cancer tissue sample (CT=34.7). Thus, expression of the gene is useful to distinguish between gastric cancer and normal tissue. Therapeutic modulation of this gene, expressed protein and/or use of antibodies or small molecule drugs targeting the gene or gene product are useful in the treatment of gastric cancer.

AI. CG55069-01, CG55069-03, CG55069-04, CG55069-09: Ten-m3.

Expression of gene CG55069-01, CG55069-03, CG55069-04, and CG55069-09 were assessed using the primer-probe sets Ag1479, Ag2674, and Ag2820, described in Tables AIA, AIB, and AIC. Results of the RTQ-PCR runs are shown in Tables AID, AIE, and AIF. CG55069-04, and CG55069-09 represent a physical clone for EGF domain. Probe-primer set Ag2820 is specific for variant CG55069-01.

TABLE AIA
Probe Name Ag1479

| | | | Start | SEQ ID |
|---|---|---|---|---|
| Primers | Sequences | Length | Position | No |
| Forward | 5′-cacggaacgtatcttcaagaaa-3′ | 22 | 2125 | 1313 |
| Probe | TET-5′-ctgcacgtgtgaccctaactggactg-3′- | 26 | 2154 | 1314 |
| | TAMRA | | | |
| Reverse | 5′-gccacagtccacagaacatatt-3′ | 22 | 2199 | 1315 |

TABLE AIB
Probe Name Ag2674

| | | | Start | SEQ ID |
|---|---|---|---|---|
| Primers | Sequences | Length | Position | No |
| Forward | 5′-acctactcggccactacctaga-3′ | 22 | 993 | 1316 |
| Probe | TET-5′-caccctatcaagaagtgcttttaaattca- | 29 | 1017 | 1317 |
| | 3′-TAMRA | | | |
| Reverse | 5′-cagtgcatttccagctacagta-3′ | 22 | 1060 | 1318 |

TABLE AIC
Probe Name Ag2820

| | | | Start | SEQ ID |
|---|---|---|---|---|
| Primers | Sequences | Length | Position | No |
| Forward | 5′-cagagaagcagacgagttcact-3′ | 22 | 354 | 1319 |
| Probe | TET-5′-caaggacagaattttaccctaaggca-3′- | 26 | 379 | 1320 |
| | TAMRA | | | |
| Reverse | 5′-gttgctggttcacaaactccta-3′ | 22 | 407 | 1321 |

TABLE AID
Panel 1.3D
Column A - Rel. Exp.(%) Ag1479, Run 165520101
Column B - Rel. Exp.(%) Ag2674, Run 162554642
Column C - Rel. Exp.(%) Ag2820, Run 165527000
Column D - Rel. Exp.(%) Ag2820, Run 165544916

| Tissue Name | A | B | C | D |
|---|---|---|---|---|
| Liver adenocarcinoma | 16.0 | 15.9 | 17.2 | 8.2 |
| Pancreas | 0.5 | 0.1 | 0.0 | 0.1 |
| Pancreatic ca. CAPAN 2 | 16.2 | 4.9 | 10.4 | 6.3 |
| Adrenal gland | 4.1 | 0.8 | 4.9 | 2.7 |
| Thyroid | 2.0 | 0.8 | 0.6 | 0.2 |
| Salivary gland | 0.2 | 0.1 | 0.0 | 0.1 |
| Pituitary gland | 3.5 | 0.6 | 0.8 | 0.1 |
| Brain (fetal) | 8.7 | 0.6 | 2.3 | 1.1 |
| Brain (whole) | 10.4 | 2.0 | 1.7 | 2.1 |
| Brain (amygdala) | 12.8 | 3.0 | 2.0 | 2.0 |
| Brain (cerebellum) | 10.0 | 1.8 | 0.3 | 0.3 |
| Brain (hippocampus) | 17.7 | 5.0 | 3.5 | 2.1 |
| Brain (substantia nigra) | 1.8 | 0.0 | 0.4 | 0.1 |
| Brain (thalamus) | 19.3 | 2.2 | 2.2 | 3.2 |
| Cerebral Cortex | 8.0 | 100.0 | 4.8 | 3.6 |
| Spinal cord | 1.4 | 1.1 | 0.4 | 1.0 |
| glio/astro U87-MG | 13.6 | 12.0 | 18.8 | 26.1 |
| glio/astro U-118-MG | 82.4 | 20.9 | 100.0 | 100.0 |
| asirocytoma SW1783 | 27.9 | 21.5 | 24.8 | 19.3 |
| neuro*; met SK-N-AS | 31.2 | 8.7 | 18.8 | 16.3 |
| astrocytoma SF-539 | 25.2 | 19.8 | 22.2 | 19.3 |
| astrocytoma SNB-75 | 20.6 | 5.2 | 27.2 | 15.7 |
| glioma SNB-19 | 4.7 | 1.6 | 4.0 | 3.4 |
| glioma U251 | 100.0 | 7.9 | 88.3 | 76.8 |
| glioma SF-295 | 5.6 | 3.3 | 5.6 | 3.5 |
| Heart (Fetal) | 1.0 | 4.3 | 0.3 | 0.3 |
| Heart | 0.7 | 0.3 | 0.0 | 0.0 |
| Skeletal muscle (Fetal) | 1.0 | 32.8 | 2.3 | 1.3 |
| Skeletal muscle | 6.0 | 2.0 | 0.0 | 0.2 |
| Bone marrow | 0.0 | 0.0 | 0.0 | 0.0 |
| Thymus | 0.2 | 0.7 | 0.5 | 0.6 |
| Spleen | 0.7 | 0.3 | 1.0 | 0.9 |
| Lymph node | 2.0 | 0.2 | 2.4 | 2.0 |
| Colorectal | 0.3 | 3.2 | 0.5 | 0.1 |
| Stomach | 3.4 | 0.1 | 2.2 | 0.1 |
| Small intestine | 3.5 | 0.6 | 1.3 | 0.7 |
| Colon ca. SW480 | 1.6 | 0.7 | 2.4 | 2.0 |
| Colon ca.* SW620 (SW480 met) | 0.0 | 0.0 | 0.0 | 0.0 |
| Colon ca. HT29 | 0.7 | 0.7 | 0.6 | 0.8 |
| Colon ca. HCT-116 | 0.3 | 0.0 | 0.0 | 0.1 |
| Colon ca. CaCo-2 | 8.6 | 14.3 | 9.7 | 7.4 |
| CC Well to Mod Duff (ODO3866) | 2.6 | 2.5 | 2.6 | 1.4 |
| Colon ca. HCC-2998 | 1.0 | 0.4 | 2.4 | 1.2 |
| Gastric ca. (liver met) NCI-N87 | 0.9 | 0.3 | 2.4 | 0.6 |
| Bladder | 0.9 | 2.5 | 2.3 | 0.4 |
| Trachea | 0.8 | 0.3 | 0.0 | 0.2 |
| Kidney | 0.8 | 0.5 | 0.0 | 0.0 |
| Kidney (fetal) | 2.8 | 1.4 | 2.5 | 1.3 |
| Renal ca. 786-0 | 11.2 | 6.4 | 19.9 | 9.5 |
| Renal ca. A498 | 13.1 | 4.3 | 13.2 | 7.2 |
| Renal ca. RXF 393 | 21.5 | 7.2 | 21.3 | 26.1 |
| Renal ca. ACHN | 10.1 | 5.1 | 7.6 | 7.5 |
| Renal ca. UO-31 | 10.2 | 3.3 | 13.8 | 9.5 |
| Renal ca. TK-10 | 0.0 | 0.0 | 0.0 | 0.0 |
| Liver | 0.0 | 0.0 | 0.0 | 0.0 |
| Liver (fetal) | 0.1 | 0.0 | 0.0 | 0.0 |
| Liver ca. (hepatoblast) HepG2 | 0.2 | 0.2 | 0.0 | 0.4 |
| Lung | 0.4 | 0.1 | 0.2 | 0.0 |
| Lung (fetal) | 0.3 | 0.3 | 0.0 | 0.7 |
| Lung ca. (small cell) LX-1 | 0.0 | 0.0 | 0.0 | 0.0 |
| Lung ca. (small cell) NCI-H69 | 3.1 | 11.6 | 5.4 | 11.2 |
| Lung ca. (s.cell var.) SHP-77 | 2.4 | 1.7 | 0.0 | 0.0 |
| Lung ca. (large cell) NCI-H460 | 18.6 | 2.6 | 26.1 | 12.9 |
| Lung ca. (non-sm. cell) A549 | 0.4 | 0.1 | 0.6 | 0.2 |
| Lung ca. (non-s.cell) NCI-H23 | 1.4 | 2.1 | 1.2 | 0.1 |
| Lung ca. (non-s.cell) HOP-62 | 9.5 | 3.9 | 16.0 | 6.8 |
| Lung ca. (non-s.cl) NCI-H522 | 28.1 | 36.9 | 15.3 | 5.8 |
| Lung ca. (squam.) SW 900 | 0.6 | 0.1 | 0.2 | 0.1 |
| Lung ca. (squam.) NGI-H596 | 16.5 | 8.0 | 19.2 | 12.3 |
| Mammary gland | 0.7 | 0.5 | 0.5 | 0.2 |
| Breast ca.* (pl.ef) MCF-7 | 5.0 | 8.8 | 5.1 | 2.1 |
| Breast ca.* (pl.ef) MDA-MB-231 | 2.4 | 0.3 | 0.5 | 0.4 |
| Breast ca.* (pl.ef) T47D | 53.6 | 26.1 | 1.9 | 1.1 |
| Breast ca. BT-549 | 0.0 | 0.0 | 0.0 | 0.0 |
| Breast ca. MDA-N | 0.8 | 1.1 | 1.5 | 1.1 |
| Ovary | 0.8 | 2.8 | 0.3 | 0.0 |
| Ovarian ca. OVCAR-3 | 58.6 | 19.3 | 26.8 | 20.0 |
| Ovarian ca. OVCAR-4 | 2.4 | 0.4 | 3.1 | 2.0 |
| Ovarian ca. OVCAR-5 | 0.0 | 0.0 | 0.0 | 0.0 |
| Ovarian ca. OVCAR-8 | 8.7 | 6.7 | 1.7 | 2.8 |
| Ovarian ca. IGROV-l | 3.1 | 1.5 | 0.0 | 0.4 |
| Ovarian ca. (ascites) SK-OV-3 | 27.9 | 6.7 | 22.2 | 0.0 |
| Uterus | 2.4 | 0.4 | 1.2 | 0.9 |
| Placenta | 8.1 | 4.4 | 7.7 | 4.1 |
| Prostate | 2.1 | 0.1 | 0.0 | 0.0 |
| Prostate ca.* (bone met) PC-3 | 0.7 | 1.1 | 0.0 | 0.0 |
| Testis | 4.5 | 1.1 | 0.0 | 0.1 |
| Melanoma Hs688(A).T | 10.0 | 20.4 | 12.8 | 7.5 |
| Melanoma* (met) Hs688(B).T | 12.5 | 18.9 | 12.0 | 4.2 |
| Melanoma UACC-62 | 1.2 | 0.3 | 0.4 | 0.3 |
| Melanoma M14 | 13.7 | 2.1 | 14.4 | 7.8 |
| Melanoma LOX IMVI | 1.2 | 1.2 | 0.0 | 0.0 |
| Melanoma* (met) SK-MEL-5 | 3.7 | 4.5 | 3.8 | 1.8 |
| Adipose | 3.6 | 4.5 | 12.9 | 0.6 |

TABLE AIE
Panel 2D
Column A - Rel. Exp. (%) Ag2674, Run 162455917
Column B - Rel. Exp. (%) Ag2820, Run 163578010
Column C - Rel. Exp. (%) Ag2820, Run 165910586

| Tissue Name | A | B | C | Tissue Name | A | B | C |
|---|---|---|---|---|---|---|---|
| Normal Colon | 47.6 | 12.4 | 15.7 | Kidney Margin 8120608 | 6.9 | 1.7 | 3.7 |
| CC Well to Mod Diff | 8.4 | 7.2 | 7.4 | Kidney Cancer 8120613 | 0.5 | 0.0 | 0.0 |
| (ODO3866) | | | | | | | |
| CC Margin (ODO3866) | 8.0 | 0.8 | 0.4 | Kidney Margin 8120614 | 2.8 | 1.6 | 0.0 |
| CC Gr.2 rectosigmoid | 5.4 | 3.8 | 2.3 | Kidney Cancer 9010320 | 22.4 | 39.5 | 36.1 |
| (ODO3868) | | | | | | | |
| CC Margin (ODO3868) | 12.4 | 2.2 | 1.2 | Kidney Margin 9010321 | 14.1 | 22.5 | 11.6 |
| CC Mod Diff (ODO3920) | 0.4 | 0.7 | 0.0 | Normal Uterus | 7.1 | 4.1 | 7.0 |
| CC Margin (ODO3920) | 12.2 | 1.6 | 1.4 | Uterine Cancer 064011 | 38.4 | 5.5 | 2.3 |
| CC Gr.2 ascend colon | 3.8 | 2.9 | 3.6 | Normal Thyroid | 13.9 | 4.7 | 1.1 |
| (ODO3921) | | | | | | | |
| CC Margin (ODO3921) | 8.9 | 1.3 | 0.0 | Thyroid Cancer | 30.4 | 36.3 | 40.9 |
| CC from Partial | 6.0 | 12.3 | 12.5 | Thyroid Cancer | 8.3 | 5.8 | 2.8 |
| Hepatectomy (ODO4309) | | | | A302152 | | | |
| Mets | | | | | | | |
| Liver Margin (ODO4309) | 0.4 | 0.4 | 0.0 | Thyroid Margin | 88.3 | 10.0 | 7.2 |
| | | | | A302153 | | | |
| Colon mets to lung | 1.4 | 1.5 | 1.1 | Normal Breast | 26.4 | 9.5 | 11.3 |
| (OD04451-01) | | | | | | | |
| Lung Margin (OD04451-02) | 0.7 | 0.0 | 0.8 | Breast Cancer | 2.0 | 0.7 | 0.8 |
| Normal Prostate 6546-1 | 14.1 | 6.3 | 2.0 | Breast Cancer | 13.7 | 4.0 | 2.9 |
| | | | | (OD04590-01) | | | |
| Prostate Cancer (OD04410) | 26.8 | 4.9 | 4.1 | Breast Cancer Mets | 55.1 | 32.5 | 15.9 |
| | | | | (OD04590-03) | | | |
| Prostate Margin (OD04410) | 27.0 | 6.0 | 1.9 | Breast Cancer | 24.8 | 12.2 | 2.9 |
| | | | | Metastasis | | | |
| Prostate Cancer (OD04720- | 18.8 | 3.2 | 1.2 | Breast Cancer | 11.2 | 7.5 | 5.5 |
| 01) | | | | | | | |
| Prostate Margin (OD04720- | 41.2 | 8.0 | 3.9 | Breast Cancer | 11.1 | 1.8 | 1.3 |
| 02) | | | | | | | |
| Normal Lung | 16.0 | 13.4 | 11.8 | Breast Cancer 9100266 | 11.8 | 3.5 | 1.2 |
| Lung Met to Muscle | 25.5 | 64.2 | 39.2 | Breast Margin 9100265 | 13.2 | 4.9 | 1.7 |
| (ODO4286) | | | | | | | |
| Muscle Margin (ODO4286) | 14.1 | 1.3 | 1.1 | Breast Cancer A209073 | 19.2 | 3.5 | 1.7 |
| Lung Malignant Cancer | 44.8 | 66.9 | 57.8 | Breast Margin A209073 | 25.3 | 0.6 | 2.0 |
| (OD03126) | | | | | | | |
| Lung Margin (OD03126) | 11.7 | 10.6 | 5.9 | Normal Liver | 1.7 | 1.2 | 0.3 |
| Lung Cancer (OD04404) | 13.7 | 10.4 | 11.6 | Liver Cancer | 0.5 | 0.0 | 0.0 |
| Lung Margin (OD04404) | 11.4 | 10.7 | 14.4 | Liver Cancer 1025 | 0.0 | 0.0 | 0.0 |
| Lung Cancer (OD04565) | 13.1 | 8.5 | 4.5 | Liver Cancer 1026 | 0.7 | 0.0 | 0.0 |
| Lung Margin (OD04565) | 3.1 | 5.3 | 6.2 | Liver Cancer 6004-T | 0.5 | 0.0 | 0.0 |
| Lung Cancer (OD04237-01) | 7.4 | 13.6 | 4.5 | Liver Tissue 6004-N | 0.6 | 1.0 | 0.3 |
| Lung Margin (OD04237-02) | 4.8 | 5.3 | 3.8 | Liver Cancer 6005-T | 1.1 | 0.0 | 0.0 |
| Ocular Mel Met to Liver | 0.9 | 0.0 | 0.0 | Liver Tissue 6005-N | 0.0 | 0.0 | 0.0 |
| (ODO4310) | | | | | | | |
| Liver Margin (ODO4310) | 5.0 | 0.0 | 0.3 | Normal Bladder | 26.1 | 14.7 | 12.7 |
| Melanoma Metastasis | 29.7 | 57.4 | 31.6 | Bladder Cancer | 6.0 | 9.2 | 2.0 |
| Lung Margin (OD04321) | 4.3 | 7.0 | 3.5 | Bladder Cancer | 6.0 | 3.9 | 2.3 |
| Normal Kidney | 27.7 | 18.9 | 14.4 | Bladder Cancer | 41.8 | 89.5 | 82.4 |
| | | | | (OD04718-01) | | | |
| Kidney Ca, Nuclear grade 2 | 2.9 | 5.6 | 2.9 | Bladder Normal | 22.4 | 3.5 | 3.9 |
| (OD04338) | | | | Adjacent (OD04718-03) | | | |
| Kidney Margin (OD04338) | 11.8 | 10.8 | 9.0 | Normal Ovary | 10.1 | 2.1 | 0.6 |
| Kidney Ca Nuclear grade 1/2 | 48.3 | 82.4 | 67.8 | Ovarian Cancer | 100.0 | 36.3 | 100.0 |
| (OD04339) | | | | | | | |
| Kidney Margin (OD04339) | 15.9 | 17.7 | 8.8 | Ovarian Cancer | 0.3 | 0.0 | 0.4 |
| | | | | (OD04768-07) | | | |
| Kidney Ca, Clear cell type | 0.8 | 0.0 | 0.3 | Ovary Margin | 8.2 | 6.9 | 4.4 |
| (OD04340) | | | | (OD04768-08) | | | |
| Kidney Margin (OD04340) | 21.6 | 13.9 | 8.0 | Normal Stomach | 5.7 | 2.2 | 1.9 |
| Kidney Ca, Nuclear grade 3 | 33.4 | 84.7 | 58.2 | Gastric Cancer 9060358 | 7.2 | 3.0 | 2.8 |
| (OD04348) | | | | | | | |
| Kidney Margin (OD04348) | 12.9 | 4.6 | 11.1 | Stomach Margin | 4.9 | 0.7 | 1.5 |
| | | | | 9060359 | | | |
| Kidney Cancer (OD04622- | 1.4 | 0.0 | 4.6 | Gastric Cancer 9060395 | 6.5 | 1.9 | 1.8 |
| 01) | | | | | | | |
| Kidney Margin (OD04622- | 7.3 | 3.9 | 1.1 | Stomach Margin | 7.2 | 2.2 | 2.3 |
| 03) | | | | 9060394 | | | |
| Kidney Cancer (OD04450- | 84.7 | 100.0 | 78.5 | Gastric Cancer 9060397 | 46.7 | 22.7 | 28.5 |
| 01) | | | | | | | |
| Kidney Margin (OD04450- | 19.9 | 12.0 | 6.9 | Stomach Margin | 4.7 | 0.7 | 0.0 |
| 03) | | | | 9060396 | | | |
| Kidney Cancer 8120607 | 12.7 | 4.9 | 4.2 | Gastric Cancer 064005 | 5.6 | 9.2 | 6.5 |

TABLE AIF
Panel 4D
Column A - Rel. Exp.(%) Ag1479, Run 162599612
Column B - Rel. Exp.(%) Ag2674, Run 160645450
Column C - Rel. Exp.(%) Ag2820, Run 162350531
Column D - Rel. Exp.(%) Ag2820, Run 164329602

| Tissue Name | A | B | C | D |
|---|---|---|---|---|
| Secondary Th1 act | 0.3 | 0.0 | 0.0 | 0.0 |
| Secondary Th2 act | 0.0 | 0.0 | 0.0 | 0.5 |
| Secondary Tr1 act | 0.0 | 0.0 | 0.0 | 0.3 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.0 | 0.0 | 0.0 |
| Primary Tr1 act | 0.0 | 0.0 | 0.0 | 0.0 |
| Primary Th1 act | 0.0 | 0.5 | 0.0 | 0.0 |
| Primary Th2 act | 0.0 | 0.0 | 0.0 | 0.0 |
| Primary Tr1 act | 0.0 | 0.0 | 0.0 | 0.0 |
| CD45RA GD4 lymphocyte act | 1.8 | 1.0 | 1.6 | 0.8 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 0.0 | 0.0 | 0.0 |
| Secondary CD8 lymphocyte act | 0.0 | 0.0 | 0.0 | 0.0 |
| CD4 lymphocyte none | 0.0 | 0.0 | 0.0 | 0.0 |
| 2ry Th1/Th2/Tr1 anti-CD95 CH11 | 0.0 | 0.0 | 0.0 | 0.0 |
| LAK cells rest | 0.0 | 0.0 | 0.0 | 0.0 |
| LAK cells IL-2 | 0.0 | 0.0 | 0.3 | 0.0 |
| LAX cells IL-2 + IL-12 | 0.0 | 0.0 | 0.0 | 0.7 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.5 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 0.0 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 0.0 | 0.0 | 0.3 | 2.5 |
| B lymphocytes CD40L and IL-4 | 0.2 | 0.4 | 0.0 | 0.0 |
| EOL-1 dbcAMP | 0.2 | 0.2 | 0.3 | 0.7 |
| EOL-1 dbcAMP PMA/ionomycin | 0.1 | 0.2 | 0.9 | 0.0 |
| Dendritic cells none | 0.0 | 0.0 | 0.0 | 0.0 |
| Dendritic cells LPS | 0.0 | 0.0 | 0.0 | 0.0 |
| Dendritic cells anti-CD40 | 0.0 | 0.0 | 0.0 | 0.0 |
| Monocytes rest | 0.0 | 0.0 | 0.0 | 0.0 |
| Monocytes LPS | 0.0 | 0.0 | 0.0 | 0.0 |
| Macrophages rest | 0.0 | 0.0 | 0.0 | 0.0 |
| Macrophages LPS | 0.0 | 0.0 | 0.0 | 0.0 |
| HUVEC none | 23.0 | 17.7 | 0.0 | 0.0 |
| HUVEC starved | 25.0 | 26.1 | 0.0 | 0.0 |
| HUVEC IL-1beta | 8.1 | 7.1 | 0.0 | 0.0 |
| HUVEC IFN gamma | 14.8 | 13.8 | 0.0 | 0.3 |
| HUVEC TNF alpha + IFN gamma | 8.5 | 6.7 | 0.0 | 0.0 |
| HUVEC TNF alpha + IL4 | 12.0 | 10.2 | 0.0 | 0.0 |
| HUVEC IL-11 | 8.5 | 7.0 | 0.0 | 0.0 |
| Lung Microvascular EC none | 11.1 | 14.2 | 0.0 | 0.0 |
| Lung Microvascular EC TNFalpha + | 9.3 | 11.0 | 0.0 | 0.2 |
| IL-1beta | | | | |
| Microvascular Dermal EC none | 100.0 | 75.3 | 0.0 | 0.0 |
| Microsvasular Dermal EC TNFalpha + | 29.7 | 26.8 | 0.0 | 0.0 |
| IL-1beta | | | | |
| Bronchial epithelium TNFalpha + IL-1beta | 0.2 | 1.3 | 2.4 | 19.9 |
| Small airway epithelium none | 2.2 | 1.1 | 1.0 | 1.7 |
| Small airway epithelium TNFalpha + | 0.3 | 0.2 | 0.0 | 0.0 |
| IL-1beta | | | | |
| Coronery artery SMC rest | 8.3 | 8.0 | 1.9 | 2.6 |
| Coronery artery SMC TNFalpha + IL-1beta | 4.6 | 3.1 | 3.0 | 1.2 |
| Astrocytes rest | 85.9 | 70.2 | 100.0 | 100.0 |
| Astrocytes TNFalpha + IL-1beta | 59.0 | 100.0 | 71.7 | 65.5 |
| KU-812 (Basophil) rest | 0.0 | 0.3 | 0.0 | 0.0 |
| KU-812 (Basophil) PMA/ionomycin | 0.0 | 0.0 | 0.0 | 0.0 |
| CCD1106 (Keratinocytes) none | 19.8 | 17.2 | 35.6 | 70.2 |
| CCD1106 (Keratinocytes) TNFalpha + | 1.7 | 1.3 | 13.4 | 29.3 |
| IL-1beta | | | | |
| Liver cirrhosis | 0.0 | 0.5 | 0.3 | 0.0 |
| Lupus kidney | 1.8 | 2.9 | 6.2 | 8.1 |
| NCI-H292 none | 0.0 | 0.0 | 0.0 | 0.0 |
| NCI-H292 IL-4 | 0.0 | 0.0 | 0.3 | 0.4 |
| NCI-H292 IL-9 | 0.0 | 0.0 | 0.0 | 0.0 |
| NCI-H292 IL-13 | 0.0 | 0.0 | 0.0 | 0.0 |
| NCI-H292 IFN gamma | 0.0 | 0.0 | 0.0 | 0.0 |
| HPAEC none | 15.1 | 12.2 | 0.0 | 0.0 |
| HPAEC TNF alpha + IL-1 beta | 6.2 | 7.5 | 0.6 | 0.0 |
| Lung fibroblast none | 0.9 | 0.4 | 0.0 | 0.4 |
| Lung fibroblast TNF alpha + IL-1 beta | 0.6 | 0.0 | 0.0 | 0.0 |
| Lung fibroblast IL-4 | 2.1 | 2.9 | 1.7 | 3.7 |
| Lung fibroblast IL-9 | 1.2 | 0.5 | 1.2 | 2.0 |
| Lung fibroblast IL-13 | 1.2 | 0.9 | 1.6 | 3.3 |
| Lung fibroblast IFN gamma | 2.1 | 1.9 | 2.3 | 0.2 |
| Dermal fibroblast CCD1070 rest | 10.5 | 9.8 | 10.3 | 8.4 |
| Dermal fibroblast CCD1070 TNF alpha | 11.6 | 4.6 | 10.0 | 11.3 |
| Dermal fibroblast CCD1070 IL-1 beta | 4.9 | 2.2 | 4.5 | 3.8 |
| Dermal fibroblast IFN gamma | 1.2 | 1.7 | 0.3 | 1.6 |
| Dermal fibroblast IL-4 | 28.3 | 27.9 | 12.1 | 13.4 |
| IBD Colitis 2 | 0.7 | 1.6 | 0.3 | 0.0 |
| IBD Crohn's | 1.6 | 0.4 | 0.8 | 3.7 |
| Colon | 8.6 | 7.6 | 1.7 | 1.9 |
| Lung | 2.0 | 2.9 | 3.8 | 6.3 |
| Thymus | 7.0 | 13.7 | 4.1 | 4.4 |
| Kidney | 17.0 | 27.5 | 13.0 | 20.2 |

This gene codes for TenM3 protein. Ten-M proteins have been shown to be involved in cell migration. This gene was upregulated in a variety of cancers. It was highly expressed in glioma, astrocytoma, lung, renal, ovarian and breast cancer cell lines. It was also expressed at high levels in primary lung, kidney, bladder, ovarian, gastric, melanoma and breast cancer tissues. This EGF repeat domain (CG55069-04 and CG55069-09 variants) of Ten-M3 is known to be involved in dimerization of the full length protein in vivo. Treatment of cells expressing Ten-M3 with this purified protein fragment will interfere with the normal function of endogenous Ten-M3 and inhibit cell migration.

Panel 1.3D Summary: Ag1479/2674/Ag2820 Highest expression of this gene was seen in the brain and in brain cancer cell lines (CTs=28-31). Thus, inhibitors of this gene or gene product is useful for the treatment of diseases involving neurite outgrowth or organization, such as neurodegenerative diseases.

There was substantial expression in other samples derived from cancer cell lines, such as breast cancer, lung cancer ovarian cancer. Thus, therapeutic modulation of this gene, encoded protein and/or use of antibodies or small molecule targeting this gene or gene product is useful in the treatment of brain cancer, lung cancer, or ovarian cancer.

This gene was also moderately expressed in metabolic tissues including adrenal, thyroid, pituitary, fetal heart, adult and fetal skeletal muscle, and adipose. Thus, this geen product may be an antibody target for the treatment of any or all diseases in these tissues, including obesity and diabetes.

Panel 2D Summary: Ag2674/2820 The highest expression of this gene is generally associated with kidney cancers. Of particular note is the consistent absence of expression in normal kidney tissue adjacent to malignant kidney. In addition, there is substantial expression associated with ovarian cancer, bladder cancer and lung cancer. Thus, the expression of this gene could be used to distinguish the above listed malignant tissue from other tissues in the panel. Particularly, the expression of this gene could be used to distinguish malignant kidney tissue from normal kidney. Moreover, therapeutic modulation of this gene, through the use of small molecule drugs, antibodies or protein therapeutics might be of benefit in the treatment of kidney cancer, ovarian cancer, bladder cancer or lung cancer.

Panel 4D Summary: Ag1479/Ag2674/Ag2820 The expression of this gene was highest in astrocytes and microvascular dermal endothelial cells (CTs=29-30), with low but significant expression in keratinocytes, and dermal fibroblasts. Expression was not modulated by any treatment, indicating that this protein is important in normal homeostasis. Therefore, modulation of this gene, expressed protein and/or use of antibodies or small molecule targeting this gene or gene product is useful in the treatmen of autoimmune and inflammatory diseases such as asthma, IBD, psoriasis, multiple sclerosis or other inflammatory diseases of the CNS.

AJ. CG55343-03: Olfactory Receptor.

Expression of gene CG55343-03 was assessed using the primer-probe sets Ag1592 and Ag457, described in Tables AJA and AJB. Results of the RTQ-PCR runs are shown in Tables AJC, AJD, AJE and AJF. CG55343-03 represents the full-length physical clone.

TABLE AJA
Probe Name Ag1592

| | | | Start | SEQ ID |
|---|---|---|---|---|
| Primers | Sequences | Length | Position | No |
| Forward | 5′-aggaccaaggaaagatggttt-3′ | 21 | 804 | 1322 |
| Probe | TET-5′-tgcacccatgctgaatccccttatat-3′- | 26 | 844 | 1323 |
| | TAMRA | | | |
| Reverse | 5′-aagccttcctttacctccttgt-3′ | 22 | 882 | 1324 |

TABLE AJB
Probe Name Ag457

| | | | Start | SEQ ID |
|---|---|---|---|---|
| Primers | Sequences | Length | Position | No |
| Forward | 5′-gccgtctctgtgtacctgca-3′ | 20 | 764 | 1325 |
| Probe | TET-5′-ttcgcccagctccaaggaccaa-3′- | 22 | 790 | 1326 |
| | TAMRA | | | |
| Reverse | 5′-ttccatagaagagagaaaccatctttc-3′ | 27 | 813 | 1327 |

TABLE AJC
Panel 1.3D
Column A - Rel. Exp. (%) Ag1592, Run 152066536
Column B - Rel. Exp. (%) Ag457, Run 146581824
Column C - Rel. Exp. (%) Ag457, Run 151104452

| Tissue Name | A | B | C | Tissue Name | A | B | C |
|---|---|---|---|---|---|---|---|
| Liver adenocarcinoma | 0.0 | 0.8 | 0.0 | Kidney (fetal) | 1.3 | 0.0 | 2.1 |
| Pancreas | 0.0 | 0.0 | 4.0 | Renal ca. 786-0 | 0.0 | 0.0 | 0.0 |
| Pancreatic ca. CAPAN 2 | 0.0 | 1.3 | 2.1 | Renal ca. A498 | 8.5 | 9.5 | 10.6 |
| Adrenal gland | 3.5 | 1.4 | 0.0 | Renal ca. RXF 393 | 0.0 | 0.0 | 0.0 |
| Thyroid | 0.0 | 0.0 | 0.9 | Renal ca. ACHN | 0.0 | 0.6 | 0.0 |
| Salivary gland | 0.0 | 0.0 | 2.3 | Renal ca. UO-31 | 0.0 | 0.0 | 0.0 |
| Pituitary gland | 0.0 | 0.0 | 0.0 | Renal ca. TK-10 | 1.7 | 1.6 | 0.9 |
| Brain (fetal) | 0.0 | 0.0 | 0.0 | Liver | 2.5 | 2.0 | 1.8 |
| Brain (whole) | 0.0 | 1.5 | 0.0 | Liver (fetal) | 6.3 | 5.7 | 12.7 |
| Brain (amygdala) | 1.4 | 1.5 | 0.0 | Liver ca. (hepatoblast) | 9.5 | 5.3 | 6.1 |
| | | | | HepG2 | | | |
| Brain (cerebellum) | 1.8 | 0.0 | 2.0 | Lung | 1.5 | 0.7 | 1.9 |
| Brain (hippocampus) | 1.2 | 0.0 | 0.0 | Lung (fetal) | 2.3 | 4.0 | 5.6 |
| Brain (substantia nigra) | 1.2 | 0.0 | 2.5 | Lung ca. (small cell) LX- | 3.4 | 2.9 | 4.0 |
| | | | | 1 | | | |
| Brain (thalamus) | 0.0 | 0.0 | 0.0 | Lung ca. (small cell) NCI- | 10.8 | 15.6 | 14.4 |
| | | | | H69 | | | |
| Cerebral Cortex | 0.0 | 1.5 | 2.1 | Lung ca. (s. cell var.) | 21.2 | 33.9 | 21.3 |
| | | | | SHP-77 | | | |
| Spinal cord | 0.0 | 0.0 | 0.0 | Lung ca. (large cell) NCI- | 5.2 | 2.0 | 2.6 |
| | | | | H460 | | | |
| glio/astro U87-MG | 4.5 | 1.5 | 1.4 | Lung ca. (non-sm. cell) | 0.0 | 3.6 | 4.5 |
| | | | | A549 | | | |
| glio/astro U-118-MG | 18.9 | 11.4 | 20.2 | Lung ca. (non-s. cell) | 18.3 | 7.6 | 14.6 |
| | | | | NCI-H23 | | | |
| astrocytoma SW1783 | 5.3 | 2.0 | 3.0 | Lung ca. (non-s. cell) | 0.0 | 0.0 | 0.0 |
| | | | | HOP-62 | | | |
| neuro*; met SK-N-AS | 7.1 | 4.5 | 14.1 | Lung ca. (non-s. cl) NCI- | 18.0 | 7.0 | 17.3 |
| | | | | H522 | | | |
| astrocytoma SF-539 | 2.6 | 0.7 | 2.9 | Lung ca. (squam.) SW | 0.0 | 1.5 | 1.5 |
| | | | | 900 | | | |
| astrocytoma SNB-75 | 9.5 | 22.7 | 17.4 | Lung ca. (squam.) NCI- | 14.0 | 17.4 | 26.2 |
| | | | | H596 | | | |
| glioma SNB-19 | 0.0 | 1.8 | 0.0 | Mammary gland | 1.6 | 0.0 | 0.0 |
| glioma U251 | 0.0 | 1.3 | 0.0 | Breast ca.* (pl. ef) MCF-7 | 100.0 | 100.0 | 100.0 |
| glioma SF-295 | 4.6 | 1.2 | 4.5 | Breast ca.* (pl. ef) MDA- | 21.0 | 24.5 | 19.8 |
| | | | | MB-231 | | | |
| Heart (Fetal) | 5.6 | 0.4 | 3.1 | Breast ca.* (pl. ef) T47D | 5.9 | 2.1 | 1.7 |
| Heart | 0.0 | 0.0 | 1.2 | Breast ca. BT-549 | 13.8 | 22.8 | 16.0 |
| Skeletal muscle (Fetal) | 4.1 | 5.0 | 6.2 | Breast ca. MDA-N | 3.5 | 3.3 | 7.7 |
| Skeletal muscle | 0.0 | 0.0 | 0.9 | Ovary | 4.1 | 0.0 | 1.0 |
| Bone marrow | 8.5 | 3.7 | 2.2 | Ovarian ca. OVCAR-3 | 8.7 | 10.5 | 5.3 |
| Thymus | 3.1 | 5.3 | 0.8 | Ovarian ca. OVCAR-4 | 0.0 | 0.0 | 0.0 |
| Spleen | 1.6 | 0.7 | 1.1 | Ovarian ca. OVCAR-5 | 11.7 | 6.3 | 4.9 |
| Lymph node | 1.1 | 1.4 | 2.3 | Ovarian ca. OVCAR-8 | 1.5 | 2.2 | 2.9 |
| Colorectal | 4.0 | 0.4 | 5.5 | Ovarian ca. IGROV-1 | 1.8 | 0.0 | 0.9 |
| Stomach | 1.4 | 1.4 | 6.5 | Ovarian ca. (ascites) SK- | 1.2 | 1.3 | 0.8 |
| | | | | OV-3 | | | |
| Small intestine | 0.0 | 0.0 | 1.7 | Uterus | 0.0 | 1.4 | 3.3 |
| Colon ca. SW480 | 27.0 | 34.2 | 34.6 | Placenta | 11.1 | 9.5 | 11.4 |
| Colon ca.* SW620 (SW480 | 2.5 | 6.1 | 11.8 | Prostate | 0.0 | 0.0 | 0.9 |
| met) | | | | | | | |
| Colon ca. HT29 | 4.1 | 1.6 | 2.1 | Prostate ca.* (bone met) | 6.5 | 5.8 | 7.2 |
| | | | | PC-3 | | | |
| Colon ca. HCT-116 | 2.8 | 0.7 | 0.0 | Testis | 1.2 | 2.1 | 1.3 |
| Colon ca. CaCo-2 | 4.6 | 6.2 | 6.3 | Melanoma Hs688(A).T | 0.0 | 0.0 | 1.3 |
| CC Well to Mod Diff | 2.4 | 0.8 | 0.0 | Melanoma* (met) | 1.8 | 0.0 | 2.2 |
| (ODO3866) | | | | Hs688(B).T | | | |
| Colon ca. HCC-2998 | 4.9 | 5.9 | 1.6 | Melanoma UACC-62 | 0.0 | 0.0 | 0.0 |
| Gastric ca. (liver met) NCI- | 42.0 | 86.5 | 65.1 | Melanoma M14 | 1.6 | 0.0 | 2.0 |
| N87 | | | | | | | |
| Bladder | 3.2 | 1.4 | 3.2 | Melanoma LOX IMVI | 0.0 | 2.6 | 2.6 |
| Trachea | 0.0 | 0.0 | 0.0 | Melanoma* (met) SK- | 0.0 | 0.0 | 0.0 |
| | | | | MEL-5 | | | |
| Kidney | 1.5 | 0.0 | 0.0 | Adipose | 0.0 | 0.0 | 0.0 |

TABLE AID
Panel 2D
Column A - Rel. Exp.(%) Ag1592, Run 152066737
Column B - Rel. Exp.(%) Ag457, Run 146087098
Column C - Rel. Exp.(%) Ag457, Run 146581925
Column D - Rel. Exp.(%) Ag457, Run 158141680

| Tissue Name | A | B | C | D |
|---|---|---|---|---|
| Normal Colon | 0.0 | 1.6 | 0.8 | 3.6 |
| CC Well to Mod Diff (ODO3866) | 1.3 | 0.5 | 0.6 | 0.0 |
| CC Margin (OD03866) | 0.0 | 2.4 | 2.0 | 2.5 |
| CC Gr.2 rectosigmoid (ODO3868) | 1.3 | 4.3 | 2.3 | 0.8 |
| CC Margin (ODO3868) | 0.9 | 0.0 | 0.7 | 1.1 |
| CC Mod Diff(ODO3920) | 4.3 | 6.3 | 6.7 | 4.0 |
| CC Margin (ODO3920) | 0.9 | 2.7 | 0.5 | 1.0 |
| CC Gr.2 ascend colon (ODO3921) | 2.7 | 1.6 | 1.7 | 0.0 |
| CC Margin (ODO3921) | 2.1 | 0.4 | 1.5 | 1.3 |
| CC from Partial Hepatectomy (ODO4309) | 3.5 | 1.8 | 1.5 | 0.6 |
| Mets | | | | |
| Liver Margin (ODO4309) | 2.0 | 0.8 | 0.8 | 2.1 |
| Colon mets to lung (OD04451-01) | 4.1 | 0.0 | 0.4 | 0.6 |
| Lung Margin (OD04451-02) | 0.0 | 0.0 | 0.7 | 0.0 |
| Normal Prostate 6546-1 | 2.2 | 3.4 | 3.1 | 1.4 |
| Prostate Cancer (OD04410) | 14.5 | 12.4 | 16.2 | 7.3 |
| Prostate Margin (OD04410) | 9.0 | 4.7 | 6.4 | 5.9 |
| Prostate Cancer (OD04720-01) | 0.0 | 0.1 | 0.4 | 1.1 |
| Prostate Margin (OD04720-02) | 0.0 | 1.0 | 0.9 | 2.9 |
| Normal Lung | 2.2 | 1.2 | 2.5 | 1.7 |
| Lung Met to Muscle (OD04286) | 3.5 | 4.2 | 3.4 | 4.2 |
| Muscle Margin (OD04286) | 0.9 | 0.0 | 0.5 | 0.0 |
| Lung Malignant Cancer (OD03126) | 0.0 | 1.5 | 1.4 | 1.7 |
| Lung Margin (OD03 126) | 0.0 | 0.9 | 2.6 | 1.5 |
| Lung Cancer (OD04404) | 6.0 | 4.1 | 3.5 | 6.8 |
| Lung Margin (OD04404) | 0.0 | 1.3 | 0.0 | 0.9 |
| Lung Cancer (OD04565) | 1.1 | 0.6 | 0.9 | 0.0 |
| Lung Margin (OD04565) | 0.0 | 1.2 | 0.4 | 0.7 |
| Lung Cancer (OD04237-01) | 17.9 | 15.9 | 14.0 | 15.7 |
| Lung Margin (OD04237-02) | 0.9 | 0.0 | 1.4 | 0.8 |
| Ocular Mel Met to Liver (ODO4310) | 0.0 | 0.3 | 0.0 | 0.0 |
| Liver Margin (ODO4310) | 0.0 | 0.7 | 1.6 | 2.9 |
| Melanoma Metastasis | 9.7 | 10.1 | 9.7 | 8.2 |
| Lung Margin (OD04321) | 0.0 | 2.3 | 1.0 | 0.0 |
| Normal Kidney | 1.6 | 0.6 | 0.7 | 0.0 |
| Kidney Ca, Nuclear grade 2 (OD04338) | 0.8 | 0.0 | 0.4 | 0.7 |
| Kidney Margin (OD04338) | 1.4 | 0.0 | 0.0 | 0.0 |
| Kidney Ca Nuclear grade 1/2 (OD04339) | 0.0 | 0.6 | 0.0 | 1.4 |
| Kidney Margin (OD04339) | 0.0 | 0.0 | 0.7 | 0.0 |
| Kidney Ca, Clear cell type (OD04340) | 3.0 | 1.8 | 3.0 | 0.9 |
| Kidney Margin (OD04340) | 0.0 | 0.9 | 0.4 | 0.0 |
| Kidney Ca, Nuclear grade 3 (OD04348) | 0.0 | 3.1 | 2.2 | 1.3 |
| Kidney Margin (OD04348) | 0.0 | 2.4 | 0.0 | 0.0 |
| Kidney Cancer (OD04622-01) | 0.0 | 0.9 | 0.8 | 1.6 |
| Kidney Margin (OD04622-03) | 0.0 | 0.8 | 0.0 | 0.0 |
| Kidney Cancer (OD04450-0l) | 0.0 | 0.7 | 0.0 | 2.7 |
| Kidney Margin (OD04450-03) | 0.9 | 0.9 | 0.8 | 0.6 |
| Kidney Cancer 8120607 | 0.0 | 0.0 | 0.4 | 0.0 |
| Kidney Margin 8120608 | 0.0 | 0.5 | 0.3 | 0.8 |
| Kidney Cancer 8120613 | 1.1 | 0.0 | 0.0 | 0.6 |
| Kidney Margin 8120614 | 0.0 | 0.0 | 0.0 | 0.0 |
| Kidney Cancer 9010320 | 0.0 | 0.4 | 1.3 | 0.0 |
| Kidney Margin 9010321 | 0.0 | 0.8 | 0.0 | 0.0 |
| Normal Uterus | 0.0 | 0.0 | 0.0 | 0.0 |
| Uterine Cancer 064011 | 0.8 | 1.2 | 2.8 | 1.6 |
| Normal Thyroid | 0.0 | 0.4 | 2.6 | 0.0 |
| Thyroid Cancer | 1.1 | 0.0 | 1.5 | 0.0 |
| Thyroid Cancer A302152 | 2.0 | 3.2 | 2.2 | 1.9 |
| Thyroid Margin A302153 | 0.8 | 0.0 | 0.7 | 0.0 |
| Normal Breast | 0.0 | 0.2 | 0.4 | 1.5 |
| Breast Cancer | 4.6 | 8.4 | 9.9 | 15.3 |
| Breast Cancer (OD04590-01) | 87.1 | 100.0 | 87.2 | 59.9 |
| Breast Cancer Mets (OD04590-03) | 36.3 | 38.7 | 55.9 | 14.8 |
| Breast Cancer Metastasis | 17.3 | 17.6 | 22.4 | 8.1 |
| Breast Cancer | 6.3 | 5.4 | 3.4 | 4.3 |
| Breast Cancer | 17.3 | 20.6 | 20.9 | 18.7 |
| Breast Cancer 9100266 | 3.8 | 5.4 | 2.7 | 3.7 |
| Breast Margin 9100265 | 3.0 | 0.8 | 0.4 | 2.5 |
| Breast Cancer A209073 | 2.2 | 2.7 | 1.3 | 0.0 |
| Breast Margin A209073 | 0.7 | 0.0 | 1.7 | 0.9 |
| Normal Liver | 1.8 | 0.5 | 0.9 | 0.0 |
| Liver Cancer | 2.0 | 0.4 | 0.8 | 0.0 |
| Liver Cancer 1025 | 1.1 | 0.7 | 0.6 | 0.0 |
| Liver Cancer 1026 | 0.0 | 0.9 | 0.0 | 0.0 |
| Liver Cancer 6004-T | 1.3 | 0.0 | 0.0 | 0.9 |
| Liver Tissue 6004-N | 3.6 | 0.8 | 1.4 | 1.1 |
| Liver Cancer 6005-T | 0.0 | 0.6 | 2.4 | 0.0 |
| Liver Tissue 6005-N | 2.3 | 0.4 | 1.2 | 1.8 |
| Normal Bladder | 7.9 | 7.1 | 3.4 | 2.0 |
| Bladder Cancer | 0.0 | 0.0 | 0.0 | 1.0 |
| Bladder Cancer | 4.2 | 4.2 | 1.8 | 11.9 |
| Bladder Cancer (OD04718-01) | 97.3 | 62.0 | 100.0 | 100.0 |
| Bladder Normal Adjacent (OD04718-03) | 0.0 | 0.9 | 0.4 | 0.0 |
| Normal Ovary | 1.1 | 0.0 | 0.0 | 0.8 |
| Ovarian Cancer | 3.4 | 1.8 | 1.1 | 3.7 |
| Ovarian Cancer (OD04768-07) | 100.0 | 76.3 | 89.5 | 85.3 |
| Ovary Margin (OD04768-08) | 1.4 | 0.0 | 0.0 | 0.0 |
| Normal Stomach | 1.1 | 0.6 | 1.4 | 0.0 |
| Gastric Cancer 9060358 | 0.0 | 0.3 | 0.0 | 0.9 |
| Stomach Margin 9060359 | 2.4 | 0.5 | 0.8 | 0.0 |
| Gastric Cancer 9060395 | 1.9 | 2.4 | 1.8 | 2.1 |
| Stomach Margin 9060394 | 2.1 | 1.9 | 0.0 | 1.7 |
| Gastric Cancer 9060397 | 10.9 | 11.7 | 10.0 | 8.7 |
| Stomach Margin 9060396 | 0.0 | 1.0 | 0.5 | 1.2 |
| Gastric Cancer 064005 | 4.9 | 0.6 | 5.6 | 2.8 |

TABLE AJE
Panel 4D
Column A - Rel. Exp. (%) Ag1592, Run 152066880
Column B - Rel. Exp. (%) Ag457, Run 151104533

| Tissue Name | A | B | Tissue Name | A | B |
|---|---|---|---|---|---|
| Secondary Th1 act | 29.9 | 8.7 | HUVEC IL-1beta | 0.0 | 3.1 |
| Secondary Th2 act | 4.7 | 3.8 | HUVEC IFN gamma | 10.2 | 7.2 |
| Secondary Tr1 act | 15.3 | 4.8 | HUVEC TNFalpha + IFN gamma | 0.0 | 0.0 |
| Secondary Th1 rest | 12.2 | 16.2 | HUVEC TNFalpha + IL4 | 0.0 | 6.8 |
| Secondary Th2 rest | 0.0 | 5.4 | HUVEC IL-11 | 0.0 | 2.0 |
| Secondary Tr1 rest | 8.3 | 0.0 | Lung Microvascular EC none | 0.0 | 12.9 |
| Primary Th1 act | 0.0 | 9.2 | Lung Microvascular EC TNFalpha + | 0.0 | 0.0 |
| | | | IL-1beta | | |
| Primary Th2 act | 4.4 | 7.5 | Microvascular Dermal EC none | 5.0 | 6.4 |
| Primary Tr1 act | 0.0 | 3.7 | Microsvasular Dermal EC TNFalpha + | 0.0 | 4.2 |
| | | | IL-1beta | | |
| Primary Th1 rest | 18.2 | 7.8 | Bronchial epithelium TNFalpha + | 0.0 | 7.9 |
| | | | IL1beta | | |
| Primary Th2 rest | 15.4 | 3.6 | Small airway epithelium none | 4.6 | 5.9 |
| Primary Tr1 rest | 2.9 | 0.0 | Small airway epithelium TNFalpha + | 5.1 | 0.0 |
| | | | IL-1beta | | |
| CD45RA CD4 lymphocyte | 0.0 | 0.0 | Coronery artery SMC rest | 0.0 | 4.1 |
| act | | | | | |
| CD45RO CD4 lymphocyte | 19.6 | 6.7 | Coronery artery SMC TNFalpha + | 0.0 | 3.5 |
| act | | | IL-1beta | | |
| CD8 lymphocyte act | 0.0 | 8.1 | Astrocytes rest | 0.0 | 7.8 |
| Secondary CD8 lymphocyte | 14.2 | 10.4 | Astrocytes TNFalpha + IL-1beta | 0.0 | 0.0 |
| rest | | | | | |
| Secondary CD8 lymphocyte | 0.0 | 3.8 | KU-812 (Basophil) rest | 3.7 | 23.2 |
| act | | | | | |
| CD4 lymphocyte none | 0.0 | 0.0 | KU-812 (Basophil) PMA/ionomycin | 100.0 | 83.5 |
| 2ry Th1/Th2/Tr1 anti-CD95 | 15.4 | 9.0 | CCD1106 (Keratinocytes) none | 0.0 | 7.0 |
| CH11 | | | | | |
| LAK cells rest | 5.1 | 0.0 | CCD1106 (Keratinocytes) TNFalpha + | 0.0 | 3.7 |
| | | | IL-1beta | | |
| LAK cells IL-2 | 5.1 | 6.8 | Liver cirrhosis | 17.1 | 44.8 |
| LAK cells IL-2 + IL-12 | 0.0 | 3.8 | Lupus kidney | 0.0 | 0.0 |
| LAK cells IL-2 + IFN | 16.6 | 0.0 | NCI-H292 none | 9.2 | 4.1 |
| gamma | | | | | |
| LAK cells IL-2 + IL-18 | 0.0 | 0.0 | NCI-H292 IL-4 | 10.4 | 4.3 |
| LAK cells PMA/ionomycin | 0.0 | 0.0 | NCI-H292 IL-9 | 23.5 | 4.1 |
| NK Cells IL-2 rest | 0.0 | 8.7 | NCI-H292 IL-13 | 0.0 | 0.0 |
| Two Way MLR 3 day | 0.0 | 2.7 | NCI-H292 IFN gamma | 6.6 | 37.6 |
| Two Way MLR 5 day | 10.9 | 3.6 | HPAEC none | 0.0 | 0.0 |
| Two Way MLR 7 day | 4.1 | 0.0 | HPAEC TNFalpha + IL-1 beta | 4.7 | 4.3 |
| PBMC rest | 5.1 | 9.6 | Lung fibroblast none | 5.5 | 6.3 |
| PBMC PWM | 12.0 | 36.1 | Lung fibroblast TNFalpha + IL-1 | 21.3 | 10.2 |
| | | | beta | | |
| PBMC PHA-L | 0.0 | 14.6 | Lung fibroblast IL-4 | 6.6 | 0.0 |
| Ramos (B cell) none | 0.0 | 10.3 | Lung fibroblast IL-9 | 0.0 | 4.3 |
| Ramos (B cell) ionomycin | 18.4 | 36.1 | Lung fibroblast IL-13 | 0.0 | 18.6 |
| B lymphocytes PWM | 31.4 | 59.0 | Lung fibroblast IFN gamma | 0.0 | 16.2 |
| B lymphocytes CD40L and | 27.7 | 12.1 | Dermal fibroblast CCD1070 rest | 18.3 | 11.7 |
| IL-4 | | | | | |
| EOL-1 dbcAMP | 7.6 | 4.5 | Dermal fibroblast CCD1O7O TNF | 19.5 | 9.8 |
| | | | alpha | | |
| EOL-1 dbcAMP | 0.0 | 0.0 | Dermal fibroblast CCD1070 IL-1 | 4.9 | 0.0 |
| PMA/ionomycin | | | beta | | |
| Dendritic cells none | 0.0 | 2.8 | Dermal fibroblast IFN gamma | 0.0 | 3.9 |
| Dendritic cells LPS | 3.5 | 4.5 | Dermal fibroblast IL-4 | 14.0 | 4.5 |
| Dendritic cells anti-CD40 | 0.0 | 3.0 | IBD Colitis 2 | 6.0 | 23.8 |
| Monocytes rest | 0.0 | 0.0 | IBD Crohn's | 0.0 | 0.0 |
| Monocytes LPS | 0.0 | 0.0 | Colon | 0.0 | 2.4 |
| Macrophages rest | 0.0 | 3.0 | Lung | 5.3 | 3.5 |
| Macrophages LPS | 1.8 | 11.7 | Thymus | 0.0 | 13.6 |
| HUVEC none | 4.2 | 0.0 | Kidney | 41.5 | 100.0 |
| HUVEC starved | 6.6 | 4.8 | | | |

TABLE AJF
general oncology screening panel_v_2.4
Column A - Rel. Exp.(%) Ag457, Run 259804332

| Tissue Name | A | Tissue Name | A |
|---|---|---|---|
| Colon cancer 1 | 1.1 | Bladder NAT 2 | 0.0 |
| CC Margin (ODO3921) | 0.0 | Bladder NAT 3 | 0.3 |
| Colon cancer 2 | 2.1 | Bladder NAT 4 | 0.0 |
| Colon NAT 2 | 0.0 | Prostate adenocarcinoma 1 | 1.0 |
| Colon cancer 3 | 2.1 | Prostate adenocarcinoma 2 | 0.0 |
| Colon NAT 3 | 0.0 | Prostate adenocarcinoma 3 | 0.8 |
| Colon malignant cancer 4 | 5.5 | Prostate adenocarcinoma 4 | 2.8 |
| Colon NAT 4 | 0.0 | Prostate NAT 5 | 0.6 |
| Lung cancer 1 | 0.0 | Prostate adenocarcinoma 6 | 2.6 |
| Lung NAT 1 | 0.0 | Prostate adenocarcinoma 7 | 2.5 |
| Lung cancer 2 | 100.0 | Prostate adenocarcinoma 8 | 1.2 |
| Lung NAT 2 | 0.4 | Prostate adenocarcinoma 9 | 1.6 |
| Squamous cell carcinoma 3 | 0.5 | Prostate NAT 10 | 0.8 |
| Lung NAT 3 | 0.0 | Kidney cancer 1 | 1.1 |
| Metastatic melanoma 1 | 0.0 | Kidney NAT 1 | 0.0 |
| Melanoma 2 | 0.6 | Kidney cancer 2 | 9.9 |
| Melanoma 3 | 0.0 | Kidney NAT 2 | 1.6 |
| Metastatic melanoma 4 | 0.6 | Kidney cancer 3 | 2.4 |
| Metastatic melanoma 5 | 1.0 | Kidney NAT 3 | 0.0 |
| Bladder cancer 1 | 0.0 | Kidney cancer 4 | 0.0 |
| Bladder NAT 1 | 0.0 | Kidney NAT 4 | 0.0 |
| Bladder cancer 2 | 0.3 | | |

Panel 1.3D Summary: Ag457/Ag1592 Expression of this gene, encoding a protein with homology to olfactory receptors, was highest in breast cancer cell line MCF-7 (CTs=32-32.8). In general, this gene was more highly expressed in cancer cell lines relative to normal tissues. Expression of this gene was significantly upregulated in 3/5 breast cancer cell lines, 2/6 ovarian cancer cell lines, and 7/10 lung cancer cell lines. Thus, expression of this gene is useful as a marker for breast, ovarian and lung cancers. Modulation of this gene, encoded protein and/or use of antibodies or small molecule drug targeting this gene or gene product is useful in the treatment of breast, ovarian and lung cancers.

Panel 2D Summary: Ag457/Ag1592 Expression of this gene was upregulated in 6/8 breast cancer samples. Expression of this gene was higher in one ovarian and one bladder cancer sample relative to the normal adjacent tissue. These results are consistent with what was observed on Panel 1.3D. Thus, expression of this gene is useful as a marker for these cancers. Modulation of this gene, encoded protein and/or use of antibodies or small molecule drug targeting this gene or gene product is useful in the treatment of breast, ovarian and bladder cancers.

Panel 4D Summary: Ag1592/Ag457 This gene showed preferential expression in activated basophils (CTs=33.9). Basophils release histamines and other biological modifiers in reponse to allergens and play an important role in the pathology of asthma and hypersensitivity reactions. These cells are a good model for the inflammatory cells that take part in various inflammatory lung and bowel diseases, such as asthma, Crohn's disease, and ulcerative colitis. Modulation of this gene, encoded protein and/or use of antibodies or small molecule drug targeting this gene or gene product is useful to reduce or eliminate the symptoms of patients suffering from asthma, Crohn's disease, and ulcerative colitis.

general oncology screening panel_v_{—}2.4 Summary: Ag457 Moderate levels of expression of this gene was detected in a lung cancer sample (CT=30.8). Low but significant expression of this gene was also seen in a kidney cancer sample. Modulation of this gene, encoded protein and/or use of antibodies or small molecule drug targeting this gene or gene product is useful in the treatment of lung and kidney cancers.

AK. CG55358-03, CG55358-04: Olfactory Receptor.

Expression of gene CG55358-03 and CG55358-04 were assessed using the primer-probe sets Ag1593, Ag455b and Ag455, described in Tables AKA, AKB and AKC. Results of the RTQ-PCR runs are shown in Tables AKD, AKE, AKF, AKG and AKH. CG55358-03 and CG55358-04 represent full-length physical clones.

TABLE AKA
Probe Name Ag1593

| | | | Start | SEQ ID |
|---|---|---|---|---|
| Primers | Sequences | Length | Position | No |
| Forward | 5′-cctagagctgatcgtctttgtg-3′ | 22 | 53 | 1328 |
| Probe | TET-5′-tcatcttttatctgctgactcttcttgg | 30 | 82 | 1329 |
| | ca-3′-TAMRA | | | |
| Reverse | 5′-gaaagcaagacaatggtcatgt-3′ | 22 | 112 | 1330 |

TABLE AKB
Probe Name Ag455b

| | | | Start | SEQ ID |
|---|---|---|---|---|
| Primers | Sequences | Length | Position | No |
| Forward | 5′-cctggctgtcatggcatatg-3′ | 20 | 329 | 1331 |
| Probe | TET-5′-tgctgcagtctgcaaacccctgc-3′- | 23 | 356 | 1332 |
| | TAMRA | | | |
| Reverse | 5′-gacgtgggtgcatgatgatg-3′ | 20 | 386 | 1333 |

TABLE AKC
Probe Name Ag455

| | | | Start | SEQ ID |
|---|---|---|---|---|
| Primers | Sequences | Length | Position | No |
| Forward | 5′-cctggctgtcatggcatatg-3′ | 20 | 329 | 1334 |
| Probe | TET-5′-ctatgctgcagtctgcaaacccctgc-3′- | 26 | 353 | 1335 |
| | TAMRA | | | |
| Reverse | 5′-gacgtgggtgcatgatgatg-3′ | 20 | 386 | 1336 |

TABLE AKD
Ardais Breast 1.0
Column A - Rel. Exp. (%) Ag1593, Run 399643616
Column B - Rel. Exp. (%) Ag455b, Run 399643480

| Tissue Name | A | B | Tissue Name | A | B |
|---|---|---|---|---|---|
| 111297 Breast cancer metastasis | 1.2 | 1.2 | 153636 Breast cancer | 0.8 | 1.2 |
| (9369)* | | | (D3D) | | |
| 108830 Breast cancer metastasis | 2.0 | 3.1 | 164668 Breast cancer | 12.5 | 14.4 |
| (OD06855)* | | | (6314) | | |
| 97764 Breast cancer node metastasis | 0.0 | 0.1 | 164677 Breast cancer | 0.6 | 0.6 |
| (OD06083) | | | (5272) | | |
| 97739 Breast cancer (CHTN20676) | 1.5 | 1.8 | 164685 Breast cancer | 0.0 | 0.4 |
| | | | (0170) | | |
| 145848 Breast cancer (9B6) | 100.0 | 100.0 | 98857 Breast cancer | 5.8 | 7.3 |
| | | | (OD06397-12) | | |
| 145849 Breast cancer (9EC) | 1.3 | 1.5 | 153628 Breast cancer | 0.5 | 0.3 |
| | | | (D35) | | |
| 153632 Breast cancer (D39) | 14.8 | 20.9 | 153637 Breast cancer | 1.6 | 1.2 |
| | | | (D3E) | | |
| 153643 Breast cancer (D44) | 0.3 | 0.1 | 164669 Breast cancer | 3.0 | 3.1 |
| | | | (6992) | | |
| 164672 Breast cancer (7464) | 12.8 | 17.6 | 164678 Breast cancer | 2.9 | 36.3 |
| | | | (5297) | | |
| 164681 Breast cancer (5787) | 1.5 | 2.1 | 164686 Breast cancer | 9.8 | 10.6 |
| | | | (0732) | | |
| 97748 Breast cancer (CHTN20931) | 0.3 | 0.1 | 145857 Breast cancer (9F0) | 1.3 | 1.3 |
| 145850 Breast cancer (9C7) | 42.0 | 57.8 | 153630 Breast cancer | 28.3 | 27.2 |
| | | | (D37) | | |
| 149844 Breast cancer (24178) | 0.5 | 0.5 | 153638 Breast cancer | 8.0 | 8.5 |
| | | | (D3F) | | |
| 153633 Breast cancer (D3A) | 1.0 | 1.9 | 164670 Breast cancer | 17.2 | 25.9 |
| | | | (7078) | | |
| 153644 Breast cancer (D45) | 0.2 | 0.2 | 164679 Breast cancer | 0.5 | 0.7 |
| | | | (5486) | | |
| 164673 Breast cancer (8452) | 6.8 | 5.8 | 164687 Breast cancer | 4.1 | 4.5 |
| | | | (5881) | | |
| 164682 Breast cancer (6342)* | 10.2 | 13.0 | 145846 Breast cancer | 0.9 | 1.0 |
| | | | (9B7) | | |
| 97751 Breast cancer (CHTN21053) | 1.8 | 2.9 | 145858 Breast cancer | 5.9 | 0.0 |
| | | | (9B4) | | |
| 116417 Breast cancer (3367)* | 1.6 | 3.2 | 153631 Breast cancer | 1.4 | 1.1 |
| | | | (D38) | | |
| 145852 Breast cancer (A1A) | 5.7 | 7.9 | 153639 Breast cancer | 1.7 | 1.5 |
| | | | (D40) | | |
| 151097 Breast cancer (CHTN24298) | 0.2 | 0.5 | 164671 Breast cancer | 6.5 | 7.3 |
| | | | (7082) | | |
| 153634 Breast cancer (D3B) | 9.8 | 14.7 | 164680 Breast cancer | 14.5 | 13.1 |
| | | | (5705) | | |
| 155797 Breast cancer (EA6) | 1.0 | 0.9 | 164688 Breast cancer | 9.9 | 10.6 |
| | | | (7222) | | |
| 164674 Breast cancer (8811) | 8.4 | 12.2 | 111288 Breast NAT (3367) | 0.1 | 0.1 |
| 164683 Breast cancer (6470) | 22.2 | 25.5 | 111302 Breast NAT (6314) | 0.5 | 0.2 |
| 97763 Breast cancer (OD06083) | 0.7 | 0.1 | 105687 Breast cancer 1B | 0.1 | 0.0 |
| 116418 Breast cancer (3378)* | 0.7 | 0.8 | 105688 Breast NAT 1A | 0.0 | 0.0 |
| 145853 Breast cancer (9F3) | 13.3 | 22.8 | 105689 Breast cancer 2B | 0.0 | 0.0 |
| 153432 Breast cancer (CHTN 24652) | 7.0 | 9.8 | 105690 Breast NAT 2A | 0.0 | 0.0 |
| 153635 Breast cancer (D3C) | 0.2 | 0.5 | 111289 Breast cancer 3B* | 7.9 | 7.9 |
| 164667 Breast cancer (5785) | 21.0 | 19.3 | 111290 Breast NAT A* | 5.6 | 7.4 |
| 164676 Breast cancer (5070) | 4.2 | 5.1 | 116424 Breast cancer 4B* | 0.1 | 0.1 |
| 164684 Breast cancer (6509) | 1.4 | 1.5 | 116425 Breast NAT 4A | 0.0 | 0.0 |
| 116421 Breast cancer (6314) | 0.1 | 0.0 | 108847 Breast cancer | 0.0 | 0.0 |
| 145854 Breast cancer (9B8) | 3.4 | 3.0 | 105694 Breast NAT | 0.3 | 0.3 |
| 153627 Breast cancer (D34) | 4.3 | 3.9 | | | |

TABLE AKE
Panel 1.3D
Column A - Rel. Exp. (%) Ag1593, Run 152078944
Column B - Rel. Exp. (%) Ag455b, Run 165974813

| Tissue Name | A | B | Tissue Name | A | B |
|---|---|---|---|---|---|
| Liver adenocarcinoma | 0.0 | 0.0 | Kidney (fetal) | 2.8 | 0.0 |
| Pancreas | 0.0 | 5.1 | Renal ca. 786-0 | 3.6 | 0.0 |
| Pancreatic ca. CAPAN 2 | 0.0 | 0.0 | Renal ca. A498 | 8.5 | 0.0 |
| Adrenal gland | 0.0 | 0.0 | Renal ca. RXF 393 | 1.8 | 0.0 |
| Thyroid | 0.0 | 0.0 | Renal ca. ACHN | 2.5 | 0.7 |
| Salivary gland | 0.0 | 0.0 | Renal ca. UO-31 | 0.0 | 0.0 |
| Pituitary gland | 0.0 | 0.0 | Renal ca. TK-10 | 0.0 | 0.0 |
| Brain (fetal) | 0.0 | 0.0 | Liver | 0.0 | 5.6 |
| Brain (whole) | 0.0 | 0.0 | Liver (fetal) | 0.0 | 0.0 |
| Brain (amygdala) | 0.0 | 0.0 | Liver ca. (hepatoblast) HepG2 | 8.7 | 3.9 |
| Brain (cerebellum) | 0.0 | 0.0 | Lung | 0.0 | 0.0 |
| Brain (hippocampus) | 0.0 | 0.0 | Lung (fetal) | 2.0 | 0.0 |
| Brain (substantia nigra) | 0.0 | 0.0 | Lung ca. (small cell) LX-1 | 4.2 | 0.0 |
| Brain (thalamus) | 0.0 | 0.0 | Lung ca. (small cell) NCI-H69 | 11.9 | 17.2 |
| Cerebral Cortex | 0.0 | 0.0 | Lung ca. (s. cell var.) SHP-77 | 28.1 | 33.4 |
| Spinal cord | 0.0 | 0.0 | Lung ca. (large cell) NCI-H460 | 3.8 | 0.0 |
| glio/astro U87-MG | 2.1 | 0.0 | Lung ca. (non-sm. cell) A549 | 1.9 | 0.0 |
| glio/astro U-118-MG | 27.7 | 14.5 | Lung ca. (non-s. cell) NCI-H23 | 24.7 | 17.1 |
| astrocytoma SW1783 | 6.0 | 0.0 | Lung ca. (non-s. cell) HOP-62 | 5.8 | 4.1 |
| neuro*; met SK-N-AS | 20.4 | 0.0 | Lung ca. (non-s. cl) NCI-H522 | 25.3 | 0.0 |
| astrocytoma SF-539 | 0.0 | 0.0 | Lung ca. (squam.) SW 900 | 0.0 | 5.0 |
| astrocytoma SNB-75 | 6.3 | 10.6 | Lung ca. (squam.) NCI-H596 | 32.8 | 31.9 |
| glioma SNB-19 | 1.7 | 0.0 | Mammary gland | 0.0 | 0.0 |
| glioma U251 | 0.0 | 0.0 | Breast ca.* (pl. ef) MCF-7 | 100.0 | 100.0 |
| glioma SF-295 | 14.3 | 6.2 | Breast ca.* (pl. ef) MDA-MB- | 25.7 | 29.1 |
| | | | 231 | | |
| Heart (Fetal) | 2.4 | 0.0 | Breast ca.* (pl. ef) T47D | 8.5 | 5.8 |
| Heart | 0.0 | 0.0 | Breast ca. BT-549 | 26.6 | 11.3 |
| Skeletal muscle (Fetal) | 3.8 | 4.7 | Breast ca. MDA-N | 6.7 | 0.0 |
| Skeletal muscle | 0.0 | 0.0 | Ovary | 0.0 | 0.0 |
| Bone marrow | 0.0 | 0.0 | Ovarian ca. OVCAR-3 | 6.5 | 17.3 |
| Thymus | 0.0 | 3.3 | Ovarian ca. OVCAR-4 | 3.6 | 0.0 |
| Spleen | 0.0 | 0.0 | Ovarian ca. OVCAR-5 | 6.1 | 9.7 |
| Lymph node | 0.0 | 0.0 | Ovarian ca. OVCAR-8 | 2.0 | 0.0 |
| Colorectal | 5.6 | 4.5 | Ovarian ca. IGROV-1 | 0.0 | 8.1 |
| Stomach | 0.0 | 0.0 | Ovarian ca. (ascites) SK-OV-3 | 2.7 | 0.0 |
| Small intestine | 2.0 | 0.0 | Uterus | 3.6 | 0.0 |
| Colon ca. SW480 | 22.4 | 8.5 | Placenta | 4.1 | 27.0 |
| Colon ca.* SW620 (SW480 met) | 8.4 | 10.8 | Prostate | 0.0 | 5.2 |
| Colon ca. HT29 | 8.2 | 0.0 | Prostate ca.* (bone met) PC-3 | 6.9 | 0.0 |
| Colon ca. HCT-116 | 1.7 | 6.5 | Testis | 9.3 | 17.1 |
| Colon ca. CaCo-2 | 7.4 | 0.0 | Melanoma Hs688(A).T | 0.0 | 0.0 |
| CC Well to Mod Diff | 3.7 | 0.0 | Melanoma* (met) Hs688(B).T | 0.0 | 0.0 |
| (ODO3866) | | | | | |
| Colon ca. HCC-2998 | 9.1 | 23.2 | Melanoma UACC-62 | 1.6 | 0.0 |
| Gastric ca. (liver met) NCI-N87 | 64.6 | 43.5 | Melanoma M14 | 0.0 | 0.0 |
| Bladder | 0.0 | 0.0 | Melanoma LOX IMVI | 2.4 | 0.0 |
| Trachea | 0.0 | 6.3 | Melanoma* (met) SK-MEL-5 | 1.9 | 0.0 |
| Kidney | 0.0 | 0.0 | Adipose | 0.0 | 0.0 |

TABLE AKF
Panel 2D
Column A - Rel. Exp. (%) Ag1593, Run 152079595
Column B - Rel. Exp. (%) Ag455, Run 145367170
Column C - Rel. Exp. (%) Ag455, Run 145492233

| Tissue Name | A | B | C | Tissue Name | A | B | C |
|---|---|---|---|---|---|---|---|
| Normal Colon | 2.5 | 0.5 | 2.5 | Kidney Margin 8120608 | 0.0 | 0.0 | 0.0 |
| CC Well to Mod Diff | 1.1 | 0.0 | 0.6 | Kidney Cancer 8120613 | 0.0 | 0.0 | 0.0 |
| (ODO3866) | | | | | | | |
| CC Margin (ODO3866) | 2.1 | 1.2 | 1.0 | Kidney Margin 8120614 | 0.0 | 0.0 | 0.0 |
| CC Gr.2 rectosigmoid | 3.2 | 1.3 | 3.2 | Kidney Cancer 9010320 | 0.9 | 0.0 | 2.5 |
| (ODO3868) | | | | | | | |
| CC Margin (ODO3868) | 0.0 | 0.0 | 0.0 | Kidney Margin 9010321 | 0.0 | 0.0 | 1.0 |
| CC Mod Diff (ODO3920) | 8.7 | 3.0 | 2.4 | Normal Uterus | 0.0 | 0.0 | 0.6 |
| CC Margin (ODO3920) | 1.9 | 0.0 | 0.0 | Uterine Cancer 064011 | 0.4 | 0.4 | 0.0 |
| CC Gr.2 ascend colon | 3.0 | 1.7 | 1.4 | Normal Thyroid | 0.0 | 0.5 | 0.6 |
| (ODO3921) | | | | | | | |
| CC Margin (ODO3921) | 0.5 | 0.4 | 0.0 | Thyroid Cancer | 0.4 | 1.5 | 0.9 |
| CC from Partial Hepatectomy | 1.7 | 2.6 | 0.0 | Thyroid Cancer A302152 | 3.0 | 2.9 | 0.8 |
| (ODO4309) Mets | | | | | | | |
| Liver Margin (ODO4309) | 0.5 | 1.2 | 0.6 | Thyroid Margin | 0.0 | 0.8 | 0.5 |
| | | | | A302153 | | | |
| Colon mets to lung | 0.0 | 0.0 | 0.5 | Normal Breast | 0.0 | 0.3 | 2.1 |
| (OD04451-01) | | | | | | | |
| Lung Margin (OD04451-02) | 0.9 | 0.0 | 0.0 | Breast Cancer | 6.2 | 7.1 | 4.9 |
| Normal Prostate 6546-1 | 0.9 | 0.5 | 1.4 | Breast Cancer | 100.0 | 49.3 | 57.0 |
| | | | | (OD04590-01) | | | |
| Prostate Cancer (OD04410) | 16.3 | 11.9 | 2.9 | Breast Cancer Mets | 37.4 | 100.0 | 40.3 |
| | | | | (OD04590-03) | | | |
| Prostate Margin (OD04410) | 5.6 | 3.8 | 0.9 | Breast Cancer Metastasis | 13.8 | 27.4 | 11.0 |
| Prostate Cancer (OD04720- | 0.5 | 1.1 | 0.0 | Breast Cancer | 3.3 | 5.1 | 0.8 |
| 01) | | | | | | | |
| Prostate Margin (OD04720- | 0.5 | 1.8 | 0.0 | Breast Cancer | 8.2 | 8.1 | 13.1 |
| 02) | | | | | | | |
| Normal Lung | 2.8 | 1.5 | 0.0 | Breast Cancer 9100266 | 1.0 | 3.0 | 1.4 |
| Lung Met to Muscle | 3.6 | 10.4 | 1.1 | Breast Margin 9100265 | 1.7 | 0.0 | 1.7 |
| (ODO4286) | | | | | | | |
| Muscle Margin (ODO4286) | 0.0 | 0.0 | 0.7 | Breast Cancer A209073 | 1.5 | 3.2 | 0.7 |
| Lung Malignant Cancer | 0.0 | 1.8 | 0.7 | Breast Margin A209073 | 0.4 | 0.8 | 1.4 |
| (OD03126) | | | | | | | |
| Lung Margin (OD03126) | 0.0 | 1.4 | 0.7 | Normal Liver | 0.0 | 2.9 | 0.0 |
| Lung Cancer (OD04404) | 2.2 | 0.7 | 2.5 | Liver Cancer | 0.0 | 0.4 | 0.9 |
| Lung Margin (OD04404) | 0.0 | 0.0 | 1.5 | Liver Cancer 1025 | 2.1 | 0.4 | 0.7 |
| Lung Cancer (OD04565) | 0.4 | 0.4 | 1.9 | Liver Cancer 1026 | 0.0 | 0.0 | 0.0 |
| Lung Margin (OD04565) | 0.0 | 0.0 | 0.0 | Liver Cancer 6004-T | 0.4 | 0.0 | 0.0 |
| Lung Cancer (OD04237-01) | 7.6 | 5.4 | 5.5 | Liver Tissue 6004-N | 4.2 | 0.7 | 2.9 |
| Lung Margin (OD04237-02) | 0.4 | 0.4 | 0.0 | Liver Cancer 6005-T | 0.7 | 0.0 | 2.4 |
| Ocular Mel Met to Liver | 0.0 | 0.0 | 0.7 | Liver Tissue 6005-N | 0.0 | 0.4 | 0.0 |
| (ODO4310) | | | | | | | |
| Liver Margin (ODO4310) | 0.0 | 0.0 | 0.0 | Normal Bladder | 3.6 | 5.4 | 0.8 |
| Melanoma Metastasis | 3.6 | 6.8 | 0.8 | Bladder Cancer | 0.7 | 0.5 | 1.8 |
| Lung Margin (OD04321) | 1.0 | 3.1 | 0.0 | Bladder Cancer | 4.5 | 2.1 | 5.4 |
| Normal Kidney | 0.0 | 0.4 | 0.0 | Bladder Cancer | 33.4 | 64.2 | 20.4 |
| | | | | (OD04718-01) | | | |
| Kidney Ca, Nuclear grade 2 | 0.9 | 0.0 | 2.3 | Bladder Normal | 0.0 | 0.7 | 0.0 |
| (OD04338) | | | | Adjacent (OD04718-03) | | | |
| Kidney Margin (OD04338) | 1.2 | 0.8 | 0.8 | Normal Ovary | 0.0 | 0.0 | 0.0 |
| Kidney Ca Nuclear grade 1/2 | 1.9 | 0.7 | 1.5 | Ovarian Cancer | 2.6 | 2.4 | 0.7 |
| (OD04339) | | | | | | | |
| Kidney Margin (OD04339) | 0.0 | 0.0 | 0.0 | Ovarian Cancer | 97.9 | 65.1 | 100.0 |
| | | | | (OD04768-07) | | | |
| Kidney Ca, Clear cell type | 0.4 | 0.0 | 0.0 | Ovary Margin | 0.0 | 0.5 | 0.0 |
| (OD04340) | | | | (OD04768-08) | | | |
| Kidney Margin (OD04340) | 0.8 | 0.0 | 0.9 | Normal Stomach | 0.0 | 1.2 | 1.5 |
| Kidney Ca, Nuclear grade 3 | 0.5 | 3.3 | 0.0 | Gastric Cancer 9060358 | 0.0 | 0.0 | 2.2 |
| (OD04348) | | | | | | | |
| Kidney Margin (OD04348) | 2.0 | 2.4 | 3.0 | Stomach Margin | 0.4 | 0.0 | 0.0 |
| | | | | 9060359 | | | |
| Kidney Cancer (OD04622- | 0.0 | 1.7 | 0.0 | Gastric Cancer 9060395 | 0.0 | 0.0 | 0.0 |
| 01) | | | | | | | |
| Kidney Margin (OD04622- | 0.4 | 0.9 | 0.0 | Stomach Margin | 0.0 | 1.0 | 0.0 |
| 03) | | | | 9060394 | | | |
| Kidney Cancer (OD04450- | 0.0 | 0.4 | 0.0 | Gastric Cancer 9060397 | 4.1 | 2.3 | 1.0 |
| 01) | | | | | | | |
| Kidney Margin (OD04450- | 0.0 | 0.0 | 0.0 | Stomach Margin | 0.0 | 1.3 | 1.1 |
| 03) | | | | 9060396 | | | |
| Kidney Cancer 8120607 | 0.0 | 0.0 | 0.0 | Gastric Cancer 064005 | 2.9 | 0.4 | 1.6 |

TABLE AKG
Panel 3D
Column A - Rd. Exp.(%) Ag455, Run 164730890

| Tissue Name | A | Tissue Name | A |
|---|---|---|---|
| 94905 Daoy | 18.2 | 94954 Ca Ski Cervical epidermoid | 33.9 |
| Medulloblastoma/Cerebellum | | carcinoma (metastasis | |
| 94906 TE671 | 0.8 | 94955 ES-2 Ovarian clear cell | 0.0 |
| Medulloblastom/Cerebellum | | carcinoma | |
| 94907 D283 Med | 3.8 | 94957 Ramos Stimulated with | 1.3 |
| Medulloblastoma/Cerebellum | | PMA/ionomycin 6h | |
| 94908 PFSK-1 Primitive | 4.5 | 94958 Ramos Stimulated with | 1.5 |
| Neuroectodermal/Cerebellum | | PMA/ionomycin 14h | |
| 94909 XF-498 CNS | 4.5 | 94962 MEG-01 Chronic | 100.0 |
| | | myelogenous leukemia | |
| | | (megokaryoblast) | |
| 94910 SNB-78 CNS/glioma | 2.0 | 94963 Raji Burkitt's lymphoma | 0.0 |
| 94911 SF-268 CNS/glioblastoma | 0.0 | 94964 Daudi Burkitt's lymphoma | 0.0 |
| 94912 T98G Glioblastoma | 7.9 | 94965 U266 B-cell | 40.3 |
| | | plasmacytomalmyeloma | |
| 96776 SK-N-SH Neuroblastoma | 0.0 | 94968 CA46 Burkitt's lymphoma | 1.6 |
| (metastasis) | | | |
| 94913 SF-295 CNS/glioblastoma | 1.9 | 94970 RL non-Hodgkin's B-cell | 0.0 |
| | | lymphoma | |
| 94914 Cerebellum | 1.2 | 94972 JM1 pre-B-cell | 3.7 |
| | | lymphoma/leukemia | |
| 96777 Cerebellum | 2.1 | 94973 Jurkat T cell leukemia | 0.0 |
| 94916 NCI-H292 Mucoepidermoid | 2.4 | 94974 TF-1 Erythroleukemia | 10.6 |
| lung carcinoma | | | |
| 94917 DMS-114 Small cell lung | 0.2 | 94975 HUT 78 T-cell lymphoma | 3.0 |
| cancer | | | |
| 94918 DMS-79 Small cell lung | 97.9 | 94977 U937 Histiocytic lymphoma | 12.2 |
| cancer/neuroendocrine | | | |
| 94919 NCI-H146 Small cell lung | 22.5 | 94980 KU-812 Myelogenous | 17.6 |
| cancer/neuroendocrine | | leukemia | |
| 94920 NCI-H526 Small cell lung | 2.3 | 769-P- Clear cell renal carcinoma | 0.0 |
| cancer/neuroendocrine | | | |
| 94921 NCI-N417 Small cell lung | 14.4 | 94983 Caki-2 Clear cell renal | 0.0 |
| cancer/neuroendocrine | | carcinoma | |
| 94923 NCI-H82 Small cell lung | 3.6 | 94984 SW 839 Clear cell renal | 1.6 |
| cancer/neuroendocnne | | carcinoma | |
| 94924 NCI-H157 Squamous cell lung | 34.2 | 94986 G401 Wilms' tumor | 0.0 |
| cancer (metastasis) | | | |
| 94925 NCI-H1155 Large cell lung | 9.8 | 94987 Hs766T Pancreatic carcinoma | 11.6 |
| cancer/neuroendocrine | | (LN metastasis) | |
| 94926 NCI-H1299 Large cell lung | 18.2 | 94988 CAPAN-1 Pancreatic | 6.6 |
| cancer/neuroendocnne | | adenocarcinoma (liver metastasis) | |
| 94927 NCI-H727 Lung carcinoid | 1.8 | 94989 SU86.86 Pancreatic carcinoma | 3.0 |
| | | (liver metastasis) | |
| 94928 NCI-UMC-11 Lung carcinoid | 7.9 | 94990 BxPC-3 Pancreatic | 13.3 |
| | | adenocarcinoma | |
| 94929 LX-1 Small cell lung cancer | 1.3 | 94991 HPAC Pancreatic | 2.8 |
| | | adenocarcinoma | |
| 94930 Colo-205 Colon cancer | 0.0 | 94992 MIA PaCa-2 Pancreatic | 0.0 |
| | | carcinoma | |
| 94931 KM12 Colon cancer | 23.7 | 94993 CFPAC-1 Pancreatic ductal | 17.7 |
| | | adenocarcinoma | |
| 94932 KM20L2 Colon cancer | 0.0 | 94994 PANC-1 Pancreatic epithelioid | 5.5 |
| | | ductal carcinoma | |
| 94933 NCI-H716 Colon cancer | 40.6 | 94996 T24 Bladder carcinma | 5.0 |
| | | (transitional cell | |
| 94935 SW-48 Colon adenocarcinoma | 1.8 | 5637- Bladder carcinoma | 0.0 |
| 94936 SW1116 Colon | 2.4 | 94998 HT-1197 Bladder carcinoma | 15.2 |
| adenocarcinoma | | | |
| 94937 LS 174T Colon | 0.0 | 94999 UM-UC-3 Bladder carcinma | 0.0 |
| adenocarcinoma | | (transitional cell) | |
| 94938 SW-948 Colon | 1.1 | 95000 A204 Rhabdomyosarcoma | 5.6 |
| adenocarcinoma | | | |
| 94939 SW-480 Colon | 0.0 | 95001 HT-1080 Fibrosarcoma | 0.0 |
| adenocarcinoma | | | |
| 94940 NCI-SNU-5 Gastric carcinoma | 11.0 | 95002 MG-63 Osteosarcoma (bone) | 2.0 |
| KATO III- Gastric carcinoma | 9.7 | 95003 SK-LMS-1 Leiomyosarcoma | 10.7 |
| | | (vulva) | |
| 94943 NCI-SNU-16 Gastric | 11.6 | 95004 SJRH30 Rhabdomyosarcoma | 1.6 |
| carcinoma | | (met to bone marrow) | |
| 94944 NCI-SNU-1 Gastric carcinoma | 6.0 | 95005 A431 Epidermoid carcinoma | 4.6 |
| 94946 RF-1 Gastric adenocarcinoma | 2.6 | 95007 WM266-4 Melanoma | 1.3 |
| 94947 RF-48 Gastric adenocarcinoma | 6.0 | DU 145- Prostate carcinoma (brain | 0.0 |
| | | metastasis) | |
| 96778 MKLN-45 Gastric carcinoma | 0.0 | 95012 MDA-MB-468 Breast | 8.5 |
| | | adenocarcinoma | |
| 94949 NCI-N87 Gastric carcinoma | 1.4 | SCC-4- Squamous cell carcinoma of | 0.0 |
| | | tongue | |
| 94951 OVCAR-5 Ovarian carcinoma | 0.0 | SCC-9- Squamous cell carcinoma of | 1.2 |
| | | tongue | |
| 94952 RL95-2 Uterine carcinoma | 3.7 | SCC-15- Squamous cell carcinoma of | 1.8 |
| | | tongue | |
| 94953 HelaS3 Cervical | 29.9 | 95017 CAL 27 Squamous cell | 0.0 |
| adenocarcinoma | | carcinoma of tongue | |

TABLE AKH
Panel 4D
Column A - Rel. Exp. (%) Ag1593, Run 152080690
Column B - Rel. Exp. (%) Ag455, Run 138083105

| Tissue Name | A | B | Tissue Name | A | B |
|---|---|---|---|---|---|
| Secondary Th1 act | 7.5 | 4.0 | HUVEC IL-1beta | 0.0 | 0.0 |
| Secondary Th2 act | 9.9 | 5.5 | HUVEC IFN gamma | 6.3 | 0.0 |
| Secondary Tr1 act | 5.0 | 7.7 | HUVEC TNF alpha + IFN gamma | 0.0 | 0.0 |
| Secondary Th1 rest | 7.2 | 5.1 | HUVEC TNF alpha +IL4 | 0.0 | 0.0 |
| Secondary Th2 rest | 5.3 | 4.6 | HUVEC IL-11 | 0.0 | 3.4 |
| Secondary Tr1 rest | 0.0 | 8.1 | Lung Microvascular EC none | 0.0 | 0.0 |
| Primary Th1 act | 0.0 | 6.6 | Lung Microvascular EC TNFalpha + | 0.0 | 1.6 |
| | | | IL-1beta | | |
| Primary Th2 act | 5.1 | 0.0 | Microvascular Dermal EC none | 0.0 | 3.6 |
| Primary Tr1 act | 0.0 | 1.5 | Microsvasular Dermal EC TNFalpha + | 7.1 | 0.0 |
| | | | IL-1beta | | |
| Primary Th1 rest | 14.8 | 6.4 | Bronchial epithelium TNFalpha + | 0.0 | 0.0 |
| | | | IL1beta | | |
| Primary Th2 rest | 4.3 | 4.1 | Small airway epithelium none | 0.0 | 3.8 |
| Primary Tr1 rest | 4.5 | 0.9 | Small airway epithelium TNFalpha + | 0.0 | 2.3 |
| | | | IL-1beta | | |
| CD45RA CD4 lymphocyte | 6.3 | 10.4 | Coronery artery SMC rest | 0.0 | 1.6 |
| act | | | | | |
| CD45RO CD4 lymphocyte | 1.6 | 3.8 | Coronery artery SMC TNFalpha + | 0.0 | 6.4 |
| act | | | IL-1beta | | |
| CD8 lymphocyte act | 0.0 | 1.5 | Astrocytes rest | 6.8 | 0.0 |
| Secondary CD8 lymphocyte | 0.0 | 1.5 | Astrocytes TNFalpha + IL-1beta | 1.7 | 6.0 |
| rest | | | | | |
| Secondary CD8 lymphocyte | 0.0 | 5.3 | KU-812 (Basophil) rest | 13.7 | 25.9 |
| act | | | | | |
| CD4 lymphocyte none | 0.0 | 0.0 | KU-812 (Basophil) PMA/ionomycin | 100.0 | 100.0 |
| 2ry Th1/Th2/Tr1 anti-CD95 | 8.8 | 5.1 | CCD1106 (Keratinocytes) none | 4.5 | 2.1 |
| CH11 | | | | | |
| LAK cells rest | 0.0 | 3.1 | CCD1106 (Keratinocytes) TNFalpha + | 0.0 | 12.1 |
| | | | IL-1beta | | |
| LAK cells IL-2 | 5.2 | 14.0 | Liver cirrhosis | 3.5 | 14.9 |
| LAK cells IL-2 + IL-12 | 1.6 | 6.7 | Lupus kidney | 0.0 | 0.0 |
| LAK cells IL-2 + IFN | 8.9 | 6.7 | NCI-H292 none | 2.5 | 3.3 |
| gamma | | | | | |
| LAK cells IL-2 + IL-18 | 7.0 | 1.2 | NCI-H292 IL-4 | 13.8 | 7.5 |
| LAK cells PMA/ionomycin | 1.6 | 0.0 | NCI-H292 IL-9 | 9.7 | 7.9 |
| NK Cells IL-2 rest | 3.8 | 5.2 | NCI-H292 IL-13 | 1.7 | 5.4 |
| Two Way MLR 3 day | 6.3 | 4.3 | NCI-H292 IFN gamma | 6.8 | 10.9 |
| Two Way MLR 5 day | 1.7 | 3.4 | HPAEC none | 0.0 | 0.0 |
| Two Way MLR 7 day | 0.0 | 1.7 | HPAEC TNFalpha + IL-1beta | 9.6 | 3.3 |
| PBMC rest | 0.0 | 1.3 | Lung fibroblast none | 3.0 | 0.0 |
| PBMC PWM | 28.1 | 25.7 | Lung fibroblast TNFalpha + IL-1 | 6.8 | 0.0 |
| | | | beta | | |
| PBMC PHA-L | 1.1 | 10.1 | Lung fibroblast IL-4 | 1.5 | 0.0 |
| Ramos (B cell) none | 6.7 | 3.6 | Lung fibroblast IL-9 | 2.9 | 0.0 |
| Ramos (B cell) ionomycin | 9.1 | 16.0 | Lung fibroblast IL-13 | 0.0 | 2.6 |
| B lymphocytes PWM | 37.6 | 47.6 | Lung fibroblast IFN gamma | 0.0 | 2.5 |
| B lymphocytes CD40L and | 6.4 | 14.4 | Dermal fibroblast CCD1070 rest | 2.5 | 12.4 |
| IL-4 | | | | | |
| EOL-1 dbcAMP | 0.0 | 2.8 | Dermal fibroblast CCD1070 TNF | 4.9 | 12.4 |
| | | | alpha | | |
| EOL-l dbcAMP | 1.2 | 1.8 | Dermal fibroblast CCD1070 IL-1 | 0.0 | 5.2 |
| PMA/ionomycin | | | beta | | |
| Dendritic cells none | 0.0 | 0.0 | Dermal fibroblast IFN gamma | 0.0 | 0.0 |
| Dendritic cells LPS | 0.0 | 0.0 | Dermal fibroblast IL-4 | 1.6 | 4.3 |
| Dendritic cells anti-CD40 | 0.0 | 0.0 | IBD Colitis 2 | 3.4 | 1.4 |
| Monocytes rest | 0.0 | 0.0 | IBD Crohn's | 1.5 | 1.6 |
| Monocytes LPS | 0.0 | 4.7 | Colon | 6.9 | 2.0 |
| Macrophages rest | 0.0 | 0.0 | Lung | 2.2 | 0.0 |
| Macrophages LPS | 0.0 | 1.7 | Thymus | 0.0 | 0.0 |
| HUVEC none | 3.0 | 0.0 | Kidney | 36.6 | 62.0 |
| HUVEC starved | 3.1 | 3.3 | | | |

Ardais Breast1.0 Summary: Ag1593/Ag455 Highest expression of this gene is detected in breast cancer 9B6 samples (CTs=25-27). Significant expression of this gene is seen in number of cancer breast cancer samples. Modulation of this gene, encoded protein and/or use of antibodies or small molecule drug targeting this gene or gene product is useful in the treatment of breast cancer.

Panel 1.3D Summary: Ag1593/Ag455b The expression of this gene was detected in a number of the cancer cell lines and not in the normal tissues. The highest expression was found in MCF-7 breast cancer cells, which are estrogen receptor positive. Low expression of this gene was also seen in cell lines derived from lung, colon, breast and gastric cancers. Modulation of this gene, encoded protein and/or use of antibodies or small molecule drug targeting this gene or gene product is useful in the treatment of breast, lung, colon and gastric cancers.

Panel 2D Summary: Ag1593/Ag455 Highest expression of this gene was detected in breast cancer samples (CTs=29-30.5). Significant expression of this gene was seen in number of cancer samples derived from breast, ovarian, bladder, colon, and lung cancers. Expression of this gene is useful as marker to detect these cancers and modulation of this gene, encoded protein and/or use of antibodies or small molecule drug targeting this gene or gene product is useful in the treatment of breast, ovarian, bladder, colon, and lung cancers.

Panel 3D Summary: Ag455 This gene was expressed widely, but at a low level, across of all of the samples in panel 3D. Highest expression was detected in a chronic myelogenous leukemia (megokaryoblast) (CT value=30), indicating a potential role for the gene in this disease.

Panel 4D Summary: Ag1593/Ag455 This gene was highly induced the KU-812 basophil cell line and was expressed at lower levels in pokeweed mitogen-activated B cells and PBMC (CTs=31-34). Activated basophils release a number of potent bioresponse modifiers that can damage surrounding tissues. Modulation of this gene, encoded protein and/or use of antibodies or small molecule drug targeting this gene or gene product will reduce or block inflammation or tissue damage caused by inflammation by blocking activation of basophils and is useful for the treatment of asthma, emphysema, and allergy.

AL. CG55604-04: Olfactory Receptor.

Expression of gene CG55604-04 was assessed using the primer-probe set Ag1240, described in Table ALA. Results of the RTQ-PCR runs are shown in Tables ALB, ALC, ALD and ALE.

TABLE ALA
Probe Name Ag1240

| | | | Start | SEQ ID |
|---|---|---|---|---|
| Primers | Sequences | Length | Position | No |
| Forward | 5′-ttccctactggggacagaatat-3′ | 22 | 504 | 1337 |
| Probe | TET-5′-tacttttgtgaacctcctgccctcct-3′- | 26 | 536 | 1338 |
| | TAMRA | | | |
| Reverse | 5′-gccatttctgtgctgtaagtgt-3′ | 22 | 579 | 1339 |

TABLE ALB
AI_comprehensive panel_v1.0
Column A - Rel. Exp.(%) Ag1240, Run 306266935

| Tissue Name | A | Tissue Name | A |
|---|---|---|---|
| 110967 COPD-F | 15.2 | 112427 Match Control Psoriasis-F | 41.2 |
| 110980 COPD-F | 26.1 | 112418 Psoriasis-M | 4.9 |
| 110968 COPD-M | 26.1 | 112723 Match Control Psoriasis-M | 29.1 |
| 110977 COPD-M | 85.9 | 112419 Psoriasis-M | 15.0 |
| 110989 Emphysema-F | 25.5 | 112424 Match Control Psonasis-M | 34.6 |
| 110992 Emphysema-F | 12.9 | 112420 Psoriasis-M | 54.3 |
| 110993 Emphysema-F | 17.9 | 112425 Match Control Psoriasis-M | 37.6 |
| 110994 Emphysema-F | 15.2 | 104689 (MF) OA Bone-Backus | 54.3 |
| 110995 Emphysema-F | 66.9 | 104690 (MF) Adj “Normal” Bone- | 31.2 |
| | | Backus | |
| 110996 Emphysema-F | 8.8 | 104691 (ME) OA Synovium-Backus | 42.3 |
| 110997 Asthma-M | 1.5 | 104692 (BA) OA Cartilage-Backus | 0.0 |
| 111001 Asthma-F | 38.4 | 104694 (BA) OA Bone-Backus | 20.4 |
| 111002 Asthma-F | 56.3 | 104695 (BA) Adj “Normal” Bone- | 28.1 |
| | | Backus | |
| 111003 Atopic Asthma-F | 50.7 | 104696 (BA) OA Synovium-Backus | 23.2 |
| 111004 Atopic Asthma-F | 70.2 | 104700 (SS) QA Bone-Backus | 15.4 |
| 111005 Atopic Asthma-F | 46.7 | 104701 (SS) Adj “Normal” Bone-Backus | 18.3 |
| 111006 Atopic Asthma-F | 9.3 | 104702 (SS) OA Synovium-Backus | 25.9 |
| 111417 Allergy-M | 44.1 | 117093 OA Cartilage Rep7 | 53.2 |
| 112347 Allergy-M | 0.0 | 112672 OA BoneS | 5.6 |
| 112349 Normal Lung-F | 0.0 | 112673 OA SynoviumS | 4.3 |
| 112357 Normal Lung-F | 20.3 | 112674 OA Synovial Fluid cells 5 | 8.6 |
| 112354 NormalLung-M | 15.1 | 117100 OA Cartilage Rep14 | 1.2 |
| 112374 Crohns-F | 54.0 | 1112756 OA Bone9 | 23.2 |
| 112389 Match Control Crohns-F | 21.3 | 112757 OA Synovium9 | 0.9 |
| 112375 Crohns-F | 26.8 | 112758 OA Synovial Fluid Cells9 | 13.2 |
| 112732 Match Control Crohns-F | 38.2 | 117125 RA Cartilage Rep2 | 5.1 |
| 112725 Crohns-M | 9.0 | 113492 Bone2 RA | 6.7 |
| 112387 Match Control Crohns-M | 5.4 | 113493 Synovium2 RA | 3.4 |
| 112378 Crohns-M | 0.2 | 113494 Syn Fluid Cells RA | 6.9 |
| 112390 Match Control Crohns-M | 60.7 | 113499 Cartilage4 RA | 4.4 |
| 112726 Crohns-M | 41.5 | 113500 Bone4 RA | 6.7 |
| 112731 Match Control Crohns-M | 13.8 | 113501 Synovium4 RA | 4.1 |
| 112380 Ulcer Col-F | 4S.1 | 113502 Syn Fluid Cells4 RA | 4.1 |
| 112734 Match Control Ulcer Col-F | 82.9 | 113495 Cartilage3 RA | 3.4 |
| 112384 Ulcer Col-F | 86.5 | 113496 Bone3 RA | 5.0 |
| 112737 Match Control Ulcer Col-F | 7.3 | 113497 Synovium3 RA | 1.9 |
| 112386 Ulcer Col-F | 12.9 | 113498 Syn Fluid Cells3 RA | 6.8 |
| 112738 Match Control Ulcer Col-F | 10.4 | 117106 Normal Cartilage Rep20 | 1.0 |
| 112381 Ulcer CoI-M | 0.0 | 113663 Bone3 Normal | 0.0 |
| 112735 Match Control Ulcer Col- | 4.4 | 113664 Synovium3 Normal | 0.0 |
| 112382 Ulcer Col-M | 24.1 | 113665 Syn Fluid Cells3 Normal | 0.7 |
| 112394 Match Control Ulcer Col- | 5.3 | 117107 Normal Cartilage Rep22 | 21.6 |
| 112383 Ulcer Col-M | 100.0 | 113667 Bone4 Normal | 32.1 |
| 112736 Match Control Ulcer Col- | 5.6 | 113668 Synovium4 Normal | 27.2 |
| M | | | |
| 112423 Psoriasis-F | 17.9 | 113669 Syn Fluid Cells4 Normal | 60.7 |

TABLE ALC
General_screening_panel_v1.4
Column A - Rel. Exp.(%) Ag1240, Run 212696338

| Tissue Name | A | Tissue Name | A |
|---|---|---|---|
| Adipose | 0.0 | Renal ca. TK-10 | 12.8 |
| Melanoma* Hs688(A).T | 2.8 | Bladder | 3.4 |
| Melanoma* Hs688(B).T | 0.9 | Gastric ca. (liver met.) | 0.0 |
| | | NCI-N87 | |
| Melanoma* M14 | 28.1 | Gastric ca. KATO III | 1.6 |
| Melanoma* LOXIMVI | 15.6 | Colon ca. SW-948 | 7.0 |
| Melanoma* SK-MEL-5 | 46.3 | Colon ca. SW480 | 65.1 |
| Squamous cell carcinoma | 6.7 | Colon ca.* (SW480 met) | 100.0 |
| SCC-4 | | SW620 | |
| Testis Pool | 63.7 | Colon ca. HT29 | 15.5 |
| Prostate ca.* (bone met) | 16.6 | Colon ca. HCT-116 | 51.4 |
| PC-3 | | | |
| Prostate Pool | 10.7 | Colon ca. CaCo-2 | 0.0 |
| Placenta | 0.0 | Colon cancer tissue | 9.9 |
| Uterus Pool | 2.6 | Colon ca. SW116 | 16.0 |
| Ovarian ca. OVCAR-3 | 52.5 | Colon ca. Colo-205 | 0.0 |
| Ovarian ca. SK-OV-3 | 54.3 | Colon ca. SW-48 | 7.3 |
| Ovarian ca. OVCAR-4 | 0.0 | Colon Pool | 26.6 |
| Ovarian ca. OVCAR-5 | 29.5 | Small Intestine Pool | 16.4 |
| Ovarian ca. IGROV-1 | 0.0 | Stomach Pool | 23.5 |
| Ovarian ca. OVCAR-8 | 8.0 | Bone Marrow Pool | 4.0 |
| Ovary | 1.6 | Fetal Heart | 3.7 |
| Breast ca. MCF-7 | 0.0 | Heart Pool | 3.4 |
| Breast ca. MDA-MB-231 | 35.6 | Lymph Node Pool | 12.3 |
| Breast ca. BT 549 | 0.0 | Fetal Skeletal Muscle | 1.2 |
| Breast ca. T47D | 26.8 | Skeletal Muscle Pool | 0.0 |
| Breast ca. MDA-N | 50.7 | Spleen Pool | 12.5 |
| Breast Pool | 53.2 | Thymus Pool | 18.9 |
| Trachea | 6.5 | CNS cancer (glio/astro) | 36.9 |
| | | U87-MG | |
| Lung | 6.5 | CNS cancer (glio/astro) | 28.9 |
| | | U-118-MG | |
| Fetal Lung | 1.3 | CNS cancer (neuro;met) | 2.4 |
| | | SK-N-AS | |
| Lung ca. NCI-N417 | 0.0 | CNS cancer (astro) SF-539 | 0.0 |
| Lung ca. LX-1 | 99.3 | CNS cancer (astro) SNB-75 | 0.0 |
| Lung ca. NCI-H146 | 18.8 | CNS cancer (glio) SNB-19 | 0.0 |
| Lung ca. SHP-77 | 1.2 | CNS cancer (glio) SF-295 | 44.4 |
| Lung ca. A549 | 3.0 | Brain (Amygdala) Pool | 12.9 |
| Lung ca. NCI-H526 | 14.6 | Brain (cerebellum) | 1.4 |
| Lung ca. NCI-H523 | 40.9 | Brain (fetal) | 26.4 |
| Lung ca. NCI-H460 | 13.5 | Brain (Hippocampus) Pool | 11.9 |
| Lung ca. HOP-62 | 11.9 | Cerebral Cortex Pool | 29.9 |
| Lung ca. NCI-H522 | 0.0 | Brain (Substantia nigra) Pool | 8.8 |
| Liver | 0.0 | Brain (Thalamus) Pool | 0.0 |
| Fetal Liver | 17.9 | Brain (whole) | 15.9 |
| Liver ca. HepG2 | 0.0 | Spinal Cord Pool | 8.3 |
| Kidney Pool | 47.6 | Adrenal Gland | 6.4 |
| Fetal Kidney | 37.6 | Pituitary gland Pool | 1.6 |
| Renal ca. 786-0 | 22.5 | Salivary Gland | 0.0 |
| Renal ca. A498 | 8.5 | Thyroid (female) | 0.0 |
| Renal ca. ACHN | 6.3 | Pancreatic ca. CAPAN2 | 0.0 |
| Renal ca. UO-31 | 17.2 | Pancreas Pool | 22.8 |

TABLE ALD
Panel 1.2
Column A - Rel. Exp.(%) Ag1240, Run 129121683

| Tissue Name | A | Tissue Name | A |
|---|---|---|---|
| Endothelial cells | 0.0 | Renal ca. 786-0 | 0.0 |
| Heart (Fetal) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal gland | 0.0 | Renal ca. UO-31 | 0.0 |
| Thyroid | 0.0 | Renal ca. TK-10 | 0.0 |
| Salivary gland | 0.0 | Liver | 0.0 |
| Pituitary gland | 0.0 | Liver (fetal) | 0.0 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) LX-1 | 39.5 |
| Brain (hippocampus) | 0.0 | Lung ca. (small cell) | 0.0 |
| | | NCI-H69 | |
| Brain (thalamus) | 0.0 | Lung ca. (s.celI var.) SHP-77 | 0.0 |
| Cerebral Cortex | 0.0 | Lung ca. (large cell) | 0.0 |
| | | NCI-H460 | |
| Spinal cord | 100.0 | Lung ca. (non-sm. cell) A549 | 0.0 |
| glio/astro U87-MG | 0.0 | Lung ca. (non.s.cell) | 0.0 |
| | | NGI-H23 | |
| glio/astro U-118-MG | 0.0 | Lung ca. (non-s.cell) HOP-62 | 0.0 |
| astrocytoma 5W1783 | 0.0 | Lung ca. (non-s.cl) NCI-H522 | 0.0 |
| neuro*; met SK-N-AS | 0.0 | Lung ca. (squam.) SW 900 | 0.0 |
| astrocytoma SF-539 | 0.0 | Lung ca. (squam.) NCI-H596 | 0.0 |
| astrocytoma SNB-75 | 0.0 | Mammary gland | 0.0 |
| glioma SNB-19 | 0.0 | Breast ca.* (pl.ef) MCF-7 | 0.0 |
| glioma U25 | 0.0 | Breast ca.* (pl.ef) | 0.0 |
| | | MDA-MB-231 | |
| glioma SF-295 | 0.0 | Breast ca.* (pl. ef) T47D | 0.0 |
| Heart | 0.0 | Breast ca. BT-549 | 0.0 |
| Skeletal muscle | 0.0 | Breast ca. MDA-N | 77.4 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 17.7 |
| Colorectal | 7.5 | Ovarian ca. OVCAR-8 | 0.0 |
| Stomach | 0.0 | Ovarian ca. IGROV-1 | 0.0 |
| Small intestine | 0.0 | Ovarian ca. (ascites) | 0.0 |
| | | SK-OV-3 | |
| Colon ca. SW480 | 0.0 | Uterus | 0.0 |
| Colon ca.* SW620 | 58.2 | Placenta | 0.0 |
| (SW480 met) | | | |
| Colon ca. HT29 | 0.0 | Prostate | 0.0 |
| Colon ca. HCT-116 | 0.0 | Prostate ca.* (bone met) PC-3 | 0.0 |
| Colon ca. CaCo-2 | 0.0 | Testis | 0.0 |
| CC Well to Mod Diff | 57.0 | Melanoma Hs688(A).T | 0.0 |
| (OD03866) | | | |
| Colon ca. HCC-2998 | 0.0 | Melanoma* (met) Hs688(B).T | 0.0 |
| Gastric ca. (liver met) | 0.0 | Melanoma UACC-62 | 0.0 |
| NCI-N87 | | | |
| Bladder | 0.0 | Melanoma M14 | 54.0 |
| Trachea | 0.0 | Melanoma LOX IMYI | 0.0 |
| Kidney | 0.0 | Melanoma* (met) SK-MEL-5 | 0.0 |
| Kidney (fetal) | 0.0 | | |

TABLE ALE
Panel 4D
Column A - Rel. Exp.(%) Ag1240, Run 140461898
Column B - Rel. Exp.(%) Ag1240, Run 145779979

| Tissue Name | A | B | Tissue Name | A | B |
|---|---|---|---|---|---|
| Secondary Th1 act | 18.9 | 15.8 | HUVEC IL-1beta | 1.6 | 11.2 |
| Secondary Th2 act | 49.3 | 62.0 | HUVEC IFN gamma | 3.8 | 6.3 |
| Secondary Tr1 act | 51.4 | 52.9 | HUVEC TNF alpha + IFN gamma | 1.8 | 7.2 |
| Secondary Th1 rest | 1.4 | 0.0 | HUVEC TNF alpha + IL4 | 7.2 | 12.4 |
| Secondary Th2 rest | 2.8 | 4.6 | HUVEC IL-11 | 3.2 | 2.1 |
| Secondary Tr1 rest | 1.4 | 7.0 | Lung Microvascular EC none | 16.5 | 17.3 |
| Primary Th1 act | 52.1 | 46.3 | Lung Microvascular EC TNFalpha + | 9.6 | 10.6 |
| | | | IL-1beta | | |
| Primary Th2 act | 39.5 | 39.5 | Microvascular Dermal EC none | 4.8 | 8.0 |
| Primary Tr1 act | 73.7 | 69.3 | Microsvasular Dermal EC TNFalpha | 1.7 | 2.2 |
| | | | + IL-1beta | | |
| Primaiy Th1 rest | 11.2 | 12.6 | Bronchial epithelium TNFalpha + | 4.6 | 0.0 |
| | | | IL1beta | | |
| Primary Th2 rest | 4.5 | 11.0 | Small airway epithelium none | 2.1 | 2.8 |
| Primary Tr1 rest | 2.9 | 9.5 | Small airway epithelium TNFalpha + | 28.5 | 26.6 |
| | | | IL-1beta | | |
| CD45RA CD4 lymphocyte | 9.1 | 7.0 | Coronery artery SMC rest | 0.5 | 1.8 |
| CD45RO CD4 lymphocyte | 23.0 | 14.6 | Coronery artery SMC TNFalpha + | 1.8 | 2.8 |
| | | | IL-1beta | | |
| CD8 lymphocyte act | 2.5 | 8.9 | Astrocytes rest | 1.2 | 0.8 |
| Secondary CD8 | 2.3 | 3.7 | Astrocytes TNFalpha + IL-1beta | 0.8 | 0.0 |
| lymphocyte rest | | | | | |
| Secondary CD8 | 15.6 | 8.2 | KU-812 (Basophil) rest | 9.2 | 7.3 |
| lymphocyte act | | | | | |
| CD4 lymphocyte none | 0.4 | 1.8 | KU-812 (Basophil) PMA/ionomycin | 33.9 | 31.4 |
| 2ry Th1/Th2/Tr1 anti- | 1.4 | 2.9 | CCD1106 (Keratinocytes) none | 11.4 | 5.6 |
| CD95 CH11 | | | | | |
| LAK cells rest | 0.0 | 0.0 | 93580 CCD1106 (Keratinocytes) | 22.7 | 0.8 |
| | | | TNFa and IFNg | | |
| LAK cells IL-2 | 0.9 | 0.0 | Liver cirrhosis | 3.5 | 8.3 |
| LAK cells IL-2 + IL-12 | 8.6 | 4.1 | Lupus kidney | 2.6 | 1.0 |
| LAK cells IL-2 + IFN | 2.5 | 2.5 | NCI-H292 none | 24.7 | 15.6 |
| gamma | | | | | |
| LAK cells IL-2 + IL-18 | 5.4 | 4.9 | NCI-H292 IL-4 | 13.8 | 34.2 |
| LAK cells PMA/ionomycin | 0.0 | 4.9 | NCI-H292 IL-9 | 15.4 | 28.7 |
| NK Cells IL-2 rest | 0.9 | 0.9 | NCI-H292 IL-13 | 8.5 | 19.3 |
| Two Way MLR 3 day | 0.0 | 3.5 | NCI-H292 IFN gamma | 4.9 | 12.8 |
| Two Way MLR 5 day | 1.2 | 2.6 | HPAEC none | 12.5 | 12.9 |
| Two Way MLR 7 day | 3.5 | 7.3 | HPAEC TNF alpha + IL-1 beta | 12.9 | 19.5 |
| PBMC rest | 0.0 | 1.6 | Lung fibroblast none | 2.9 | 2.9 |
| PBMC PWM | 3.4 | 3.8 | Lung fibroblast TNF alpha + IL-1 | 2.0 | 1.0 |
| | | | beta | | |
| PBMC PHA-L | 12.2 | 10.4 | Lung fibroblast IL-4 | 4.0 | 2.9 |
| Ramos (B cell) none | 82.9 | 50.7 | Lung fibroblast IL-9 | 8.2 | 6.0 |
| Ramos (B cell) ionomycin | 100.0 | 100.0 | Lung fibroblast IL-13 | 5.0 | 3.3 |
| B lymphocytes PWM | 3.6 | 7.0 | Lung fibroblast IFN gamma | 2.5 | 5.6 |
| B lymphocytes CD40L and | 4.3 | 7.9 | Dermal fibroblast CCD1070 rest | 29.1 | 35.4 |
| IL-4 | | | | | |
| EOL-1 dbcAMP | 0.0 | 0.0 | Dermal fibroblast CCD1070 TNF | 42.6 | 57.0 |
| | | | alpha | | |
| EOL-1 dbcAMP | 0.0 | 0.0 | Dermal fibroblast CCD1070 IL-1 | 17.8 | 31.4 |
| PMA/ionomycin | | | beta | | |
| Dendritic cells none | 0.0 | 0.0 | Dermal fibroblast IFN gamma | 0.9 | 0.8 |
| Dendritic cells LPS | 0.0 | 42.9 | Dermal fibroblast IL-4 | 1.8 | 3.1 |
| Dendritic cells anti-CD40 | 0.0 | 0.0 | IBD Colitis 2 | 1.8 | 0.6 |
| Monocytes rest | 0.0 | 0.0 | IBD Crohn's | 0.0 | 0.9 |
| Monocytes LPS | 0.0 | 0.0 | Colon | 0.9 | 2.2 |
| Macrophages rest | 7.6 | 4.5 | Lung | 1.8 | 5.3 |
| Macrophages LPS | 1.2 | 0.0 | Thymus | 16.6 | 7.0 |
| HUVEC none | 11.5 | 11.3 | Kidney | 17.2 | 18.8 |
| HUVEC starved | 14.2 | 19.8 | | | |

AI_comprehensive panel_v1.0 Summary: Ag1240 The highest expression of this gene was detected in an ulcerative colitis sample. Moderate levels of expression of this gene were detected in samples derived from normal and orthoarthitis/rheumatoid arthritis bone and adjacent bone, cartilage, synovium and synovial fluid samples, from normal lung, COPD lung, emphysema, atopic asthma, asthma, allergy, Crohn's disease (normal matched control and diseased), ulcerative colitis (normal matched control and diseased), and psoriasis (normal matched control and diseased). Therapeutic modulation of this gene, expressed protein and/or use of antibodies or small molecule drugs targeting the gene or gene product are useful in the treatment of autoimmune and inflammatory disorders including psoriasis, allergy, asthma, inflammatory bowel disease, rheumatoid arthritis and osteoarthritis.

General_screening_panel_v1.4 Summary: Ag1240 The highest expression of this gene was detected in a colon cancer cell line (CT=31). Significant expression of this gene was seen in pancreas, melanoma, lymph node, spleen, thymus, brain, testis, prostate (both normal and cancer), breast (both normal and cancer), kidney (fetal, and adult normal and cancer). The expression of this gene was upregulated in several ovarian and lung cancer cell lines, and downregulated in stomach and breast cancer cell lines. Therapeutic modulation of this gene, expressed protein and/or use of antibodies or small molecule drugs targeting the gene or gene product are useful in the treatment of ovarian, lung, stomach and breast cancers.

Panel 1.2 Summary: Ag1240 The highest exoression of this gene was detected in spinal cord. Therapeutic modulation of this gene, expressed protein and/or use of antibodies or small molecule drugs targeting the gene or gene product are useful in the treatment of disorders and injuries of the spinal cord.

Panel 4D Summary: Ag1240 The highest expression of this gene was seen in the B cell lymphoma Ramos (CT=29). This gene was highly expressed in activated T cells, particularly in activated T cells which have been cultured under conditions which skew their development into Th1, Th2 or Tr1 cells, but not in resting T cells. Therapeutic modulation of this gene, expressed protein and/or use of antibodies or small molecule drugs targeting the gene or gene product are useful in the treatment of inflammation resulting from T cell activation and T cell-mediated autoimmune diseases such as arthritis, Crohn's disease, asthma/allergy, diabetes and psoriasis. These therapeutics is also important in preventing organ rejection due to T cell activation.

AM. CG55752406 and CG55752-07: Glucosidase.

Expression of genes CG55752-06 and CG55752-07 were assessed using the primer-probe set Ag401, described in Table AMA. Results of the RTQ-PCR runs are shown in Tables AMB and AMC.

TABLE AMA
Probe Name Ag401

| | | | Start | SEQ ID |
|---|---|---|---|---|
| Primers | Sequences | Length | Position | No |
| Forward | 5′-ttgtgccaaaacatccatcct-3′ | 21 | 1896 | 1340 |
| Probe | TET-5′-agcctggagaagctctcactcaacattgc- | 29 | 1918 | 1341 |
| | 3′-TAMRA | | | |
| Reverse | 5′-tatgatgcggacctcccagt-3′ | 20 | 1952 | 1342 |

TABLE AMB
Panel 1.3D
Column A - Rel. Exp.(%) Ag401, Run 165518174

| Tissue Name | A | Tissue Name | A |
|---|---|---|---|
| Liver adenocarcinoma | 2.9 | Kidney (fetal) | 13.8 |
| Pancreas | 10.1 | Renal ca. 786-0 | 14.8 |
| Pancreatic ca. CAPAN 2 | 10.2 | Renal ca. A498 | 13.9 |
| Adrenal gland | 16.2 | Renal ca. RXF 393 | 18.2 |
| Thyroid | 18.9 | Renal ca. ACHN | 12.7 |
| Salivary gland | 24.7 | Renal ca. UO-31 | 12.2 |
| Pituitary gland | 20.2 | Renal ca. TK-10 | 5.7 |
| Brain (fetal) | 23.5 | Liver | 16.7 |
| Brain (whole) | 52.1 | Liver (fetal) | 90 |
| Brain (amygdala) | 16.2 | Liver ca. (hepatoblast) HepG2 | 9.6 |
| Brain (cerebellum) | 47.6 | Lung | 19.5 |
| Brain (hippocampus) | 54.3 | Lung (fetal) | 11.2 |
| Brain (substantia nigra) | 20.0 | Lung ca. (small cell) LX-1 | 18.7 |
| Brain (thalamus) | 41.5 | Lung ca. (small cell) NCI-H69 | 0.0 |
| Cerebral Cortex | 15.0 | Lung ca. (s.cell var.) SHP-77 | 10.9 |
| Spinal cord | 38.7 | Lung ca. (large cell) NCI-H460 | 19.5 |
| glio/astro U87-MG | 9.5 | Lung ca. (non-sm. cell) A549 | 1.6 |
| glio/astro U-118-MG | 49.7 | Lung ca. (non-s.cell) NCI-H23 | 9.3 |
| astrocytoma SW1783 | 10.4 | Lung ca. (non-s.cell) HOP-62 | 17.8 |
| neuro*; met SK-N-AS | 20.2 | Lung ca. (non-s.cl) NCI-H522 | 9.0 |
| astrocytoma SF-539 | 25.7 | Lung ca. (squam.) SW 900 | 4.5 |
| astrocytoma SNB-75 | 22.4 | Lung ca. (squam.) NCI-H596 | 3.1 |
| glioma SNB-19 | 25.9 | Mammary gland | 27.0 |
| glioma U251 | 59.9 | Breast ca.* (pl.ef) MCF-7 | 4.3 |
| glioma SF-295 | 15.4 | Breast ca.* (pl.ef) | 47.3 |
| | | MDA-MB-231 | |
| Heart (Fetal) | 0.5 | Breast ca.* (pl.ef) T47D | 6.4 |
| Heart | 14.0 | Breast ca. BT-549 | 12.9 |
| Skeletal muscle (Fetal) | 7.0 | Breast ca. MDA-N | 6.2 |
| Skeletal muscle | 100.0 | Ovary | 6.3 |
| Bone marrow | 33.9 | Ovarian ca. OVCAR-3 | 10.5 |
| Thymus | 32.1 | Ovarian ca. OVCAR-4 | 7.2 |
| Spleen | 27.9 | Ovarian ca. OVCAR-5 | 13.6 |
| Lymph node | 76.8 | Ovarian ca. OVCAR-8 | 4.6 |
| Colorectal | 38.7 | Ovarian ca. IGROV-1 | 2.5 |
| Stomach | 21.0 | Ovarian ca. (ascites) SK-OV-3 | 17.3 |
| Small intestine | 60.7 | Utems | 61.6 |
| Colon ca. SW480 | 7.6 | Placenta | 2.9 |
| Colon ca.* SW620 | 2.9 | Prostate | 17.8 |
| (SW480 met) | | | |
| Colon ca. HT29 | 0.8 | Prostate ca.* (bone met) PC-3 | 19.6 |
| Colon ca. HCT-116 | 3.6 | Testis | 50.7 |
| Colon ca. CaCo-2 | 3.1 | Melanoma Hs688(A).T | 9.5 |
| CC Well to Mod Diff | 7.9 | Melanoma* (met) Hs688(B).T | 15.8 |
| (OD03866) | | | |
| Colon ca. HCC-2998 | 10.3 | Melanoma UACC-62 | 7.6 |
| Gastric ca. (liver met) | 55.1 | Melanoma M14 | 89.5 |
| NCI-N87 | | | |
| Bladder | 22.2 | Melanoma LOX IMVI | 2.2 |
| Trachea | 17.4 | Melanoma* (met) SK-MEL-5 | 1.9 |
| Kidney | 19.5 | Adipose | 15.5 |

TABLE AMC
Panel 5 Islet
Column A - Rel. Exp.(%) Ag401, Run 304686255

| Tissue Name | A | Tissue Name | A |
|---|---|---|---|
| 97457 Patient-02go adipose | 1.5 | 94709 Donor 2 AM - A adipose | 16.8 |
| 97476 Patient-07sk skeletal muscle | 0.0 | 94710 Donor 2 AM - B adipose | 11.3 |
| 97477 Patient-07ut uterus | 2.8 | 94711 Donor 2 AM - C adipose | 14.2 |
| 97478 Patient-07pl placenta | 0.8 | 94712 Donor 2 AD - A adipose | 28.1 |
| 99167 Bayer Patient 1 | 0.0 | 94713 Donor 2 AD - B adipose | 50.7 |
| 97482 Patient-08ut uterus | 0.0 | 94714 Donor 2 AD - C adipose | 25.0 |
| 97483 Patient-08pl placenta | 2.2 | 94742 Donor 3 U - A Mesenchymal | 8.2 |
| | | Stem Cells | |
| 97486 Patient-09sk skeletal muscle | 10.7 | 94743 Donor 3 U - B Mesenchymal | 5.2 |
| | | Stem Cells | |
| 97487 Patient-09ut uterus | 7.8 | 94730 Donor 3 AM - A adipose | 33.4 |
| 97488 Patient-09pl placenta | 2.0 | 94731 Donor 3 AM - B adipose | 100.0 |
| 97492 Patient-10ut uterus | 7.0 | 94732 Donor 3 AM - C adipose | 52.5 |
| 97493 Patient-10pl placenta | 9.3 | 94733 Donor 3 AD - A adipose | 47.6 |
| 97495 Patient-11go adipose | 24.1 | 94734 Donor 3 AD - B adipose | 29.5 |
| 97496 Patient-11sk skeletal muscle | 8.2 | 94735 Donor 3 AD - C adipose | 10.7 |
| 97497 Patient-11ut uterus | 18.2 | 77138 Liver HepG2untreated | 46.3 |
| 97498 Patient-11pl placenta | 0.0 | 73556 Heart Cardiac stromal cells | 12.0 |
| | | (primary) | |
| 97500 Patient-12go adipose | 25.2 | 81735 Small Intestine | 34.6 |
| 97501 Patient-12sk skeletal muscle | 17.9 | 72409 Kidney Proximal Convoluted | 70.2 |
| | | Tubule | |
| 97502 Patient-12ut uterus | 16.2 | 82685 Small intestine Duodenum | 9.9 |
| 97503 Patient-12pl placenta | 6.0 | 90650 Adrenal Adrenocortical | 5.6 |
| | | adenoma | |
| 94721 Donor 2 U - A Mesenchymal | 25.2 | 72410 Kidney HRCE | 44.1 |
| 94722 Donor 2 U - B Mesenchymal | 9.5 | 72411 Kidney HRE | 14.5 |
| Stem Cells | | | |
| 94723 Donor 2 U - C Mesenchymal | 19.1 | 73139 Uterus Uterine smooth muscle | 37.1 |
| Stem Cells | | cells | |

Panel 1.3D Summary: Ag401 The highest expression of this gene was detected in skeletal muscle. It was expressed in a variety of metabolic tissues, including pancreas, adipose, adrenal, thyroid, pituitary, adult and fetal heart, adult and fetal skeletal muscle, and adult and fetal liver. This gene encodes an alpha-glucosidase. Alpha-glucosidase inhibitors are currently used in the treatment of Type 2 diabetes to decrease glucose absorption from the gut (Raptis S A, Dimitriadis G D. Oral hypoglycemic agents: insulin secretagogues, alpha-glucosidase inhibitors and insulin sensitizers. Exp Clin Endocrinol Diabetes. 2001; 109 Suppl 2:S265-87). Thus, this gene, encoded protein and/or use of small molecule targeting this gene or gene product is useful for the treatment of metabolic diseases, including obesity and Types 1 and 2 diabetes.

This gene was expressed in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product are useful in the treatment of central nervous system disorders, such as Alzheimer's disease, Parkinson's disease, epilepsy, multiple sclerosis, schizophrenia and depression.

Panel 5 Islet Summary: Ag401 The highest expression of this gene was detected during adipocyte differention (CT=32). This gene was upregulated during adipocyte differentiation. It was expressed in a variety of metabolic tissues, including adipose, heart, small intestine, kidney, uterus, skeletal muscle. Therapeutic modulation of this gene, expressed protein and/or use of antibodies or small molecule drugs targeting the gene or gene product are useful in the treatment of obesity and diabetes.

AN. CG55778-03 and CG55778-06: Aldo-Keto Reductase.

Expression of gene CG55778-03 and CG55778-06 were assessed using the primer-probe set Ag7193, described in Table ANA. Results of the RTQ-PCR runs are shown in Table ANB. CG55778-03 and CG55778-06 represent full length physical clones.

TABLE ANA
Probe Name Ag7193

| | | | Start | SEQ ID |
|---|---|---|---|---|
| Primers | Sequences | Length | Position | No |
| Forward | 5′-gacacgtgggagattttgatc-3′ | 21 | 473 | 1343 |
| Probe | TET-5′-agtgatccccggatctatcaccccaa-3′- | 26 | 520 | 1344 |
| | TAMRA | | | |
| Reverse | 5′-acacctggatattctctttaatgtga-3′ | 26 | 547 | 1345 |

TABLE ANB
General_screening_panel_v1.7
Column A - Rel. Exp.(%) Ag7193, Run 318040434

| Tissue Name | A | Tissue Name | A |
|---|---|---|---|
| Adipose | 5.0 | Gastric ca. (liver met.) NCI-N87 | 0.0 |
| HUVEC | 1.9 | Stomach | 0.2 |
| Melanoma* Hs688(A).T | 0.0 | Colon ca. SW-948 | 0.4 |
| Melanoma* Hs688(B).T | 4.5 | Colon ca. SW480 | 0.0 |
| Melanoma (met) SK-MEL-5 | 0.5 | Colon ca. (SW480 met) SW620 | 12.9 |
| Testis | 3.7 | Colon ca. HT29 | 1.6 |
| Prostate ca. (bone met) PC-3 | 0.0 | Colon ca. HCT-116 | 1.2 |
| Prostate ca. DU145 | 1.4 | Colon cancer tissue | 0.0 |
| Prostate pool | 0.5 | Colon ca. SW1116 | 0.0 |
| Uterus pool | 0.6 | Colon ca. Colo-205 | 0.0 |
| Ovarian ca. OVCAR-3 | 0.1 | Colon ca. SW-48 | 1.8 |
| Ovarian ca. (ascites) SK-OV-3 | 0.2 | Colon | 1.1 |
| Ovarian ca. OVCAR-4 | 2.0 | Small Intestine | 0.3 |
| Ovarian ca. OVCAR-5 | 2.7 | Fetal Heart | 0.9 |
| Ovarian ca. IGROV-1 | 0.0 | Heart | 0.2 |
| Ovarian ca. OVCAR-8 | 3.5 | Lymph Node Pool | 0.3 |
| Ovary | 1.2 | Lymph Node pool 2 | 2.1 |
| Breast ca. MCF-7 | 2.3 | Fetal Skeletal Muscle | 0.8 |
| Breast ca. MDA-MB-231 | 3.1 | Skeletal Muscle pool | 0.1 |
| Breast ca. BT 549 | 0.4 | Skeletal Muscle | 0.6 |
| Breast ca. T47D | 1.1 | Spleen | 1.4 |
| 113452 mammary gland | 0.4 | Thymus | 0.8 |
| Trachea | 2.4 | CNS cancer (glio/astro) SF-268 | 0.0 |
| Lung | 9.5 | CNS cancer (glio/astro) T98G | 0.0 |
| Fetal Lung | 2.1 | CNS cancer (neuro;met) SK-N-AS | 0.0 |
| Lung ca. NCI-N417 | 0.0 | CNS cancer (astro) SF-539 | 0.0 |
| Lung ca. LX-1 | 3.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.9 | CNS cancer (glio) SF-295 | 0.4 |
| Lung ca. NCI-H23 | 0.5 | Brain (Amygdala) | 0.8 |
| Lung ca. NCI-H460 | 0.3 | Brain (Cerebellum) | 5.3 |
| Lung ca. HOP-62 | 10.0 | Brain (Fetal) | 8.1 |
| Lung ca. NCI-H522 | 100.0 | Brain (Hippocampus) | 1.1 |
| Lung ca. DMS-114 | 7.6 | Cerebral Cortex pool | 1.0 |
| Liver | 0.2 | Brain (Substantia nigra) | 0.4 |
| Fetal Liver | 0.6 | Brain (Thalamus) | 0.8 |
| Kidney pool | 2.7 | Brain (Whole) | 4.6 |
| Fetal Kidney | 1.2 | Spinal Cord | 0.4 |
| Renal ca. 786-0 | 0.1 | Adrenal Gland | 1.7 |
| Renal ca. A498 | 1.2 | Pituitary Gland | 1.3 |
| Renal ca. ACHN | 0.2 | Salivary Gland | 0.8 |
| Renal ca. UO-31 | 0.2 | Thyroid | 4.6 |
| Renal ca. TK-10 | 0.0 | Pancreatic ca. PANC-1 | 0.3 |
| Bladder | 1.8 | Pancreas pool | 0.3 |

General_screening_panel_v1.7 Summary: Ag7193 Among tissues with metabolic or endocrine function, this gene was expressed at high to moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product are useful in the treatment of endocrine/metabolically related diseases, such as obesity and diabetes.

AO. CG55794-03: Novel Carboxypeptidase B Like Protein.

Expression of gene CG55794-03 was assessed using the primer-probe sets Ag2622 and Ag3953, described in Tables AOA and AOB. Results of the RTQ-PCR runs are shown in Tables AOC, AOD, AOE and AOF. This gene represents a full-length physical clone.

TABLE AOA
Probe Name Ag2622

| | | | Start | SEQ ID |
|---|---|---|---|---|
| Primers | Sequences | Length | Position | No |
| Forward | 5′-catcagggtcttcaagagattg-3′ | 22 | 909 | 1346 |
| Probe | TET-5′-ccgagacattgggattcccttctcat-3′- | 26 | 934 | 1347 |
| | TAMRA | | | |
| Reverse | 5′-acaaacccatatgttccactgt-3′ | 22 | 978 | 1348 |

TABLE AOB
Probe Name Ag3953

| | | | Start | SEQ ID |
|---|---|---|---|---|
| Primers | Sequences | Length | Position | No |
| Forward | 5′-acagtggaacatatgggtttgt-3′ | 22 | 978 | 1349 |
| Probe | TET-5′-agaagctcagatccagcccacctgt-3′- | 25 | 1006 | 1350 |
| | TAMRA | | | |
| Reverse | 5′-catacacatcatccaggactga-3′ | 22 | 1055 | 1351 |

TABLE AOC
Panel 1.3D
Column A - Rel. Exp.(%) Ag2622, Run 162554681
Column B - Rel. Exp.(%) Ag2622, Run 165672349

| Tissue Name | A | B | Tissue Name | A | B |
|---|---|---|---|---|---|
| Liver adenocarcinoma | 0.0 | 0.0 | Kidney (fetal) | 10.5 | 0.0 |
| Pancreas | 0.0 | 0.0 | Renal ca. 786-0 | 0.0 | 0.0 |
| Pancreatic ca. CAPAN 2 | 20.0 | 10.3 | Renal ca. A498 | 7.0 | 22.2 |
| Adrenal gland | 7.4 | 33.7 | Renal ca. RXF 393 | 0.0 | 8.5 |
| Thyroid | 46.7 | 7.7 | Renal ca. ACHN | 0.0 | 0.0 |
| Salivary gland | 0.0 | 21.2 | Renal ca. UO-31 | 0.0 | 0.0 |
| Pituitary gland | 14.8 | 40.9 | Renal ca. TK-10 | 0.0 | 0.0 |
| Brain (fetal) | 13.8 | 0.0 | Liver | 0.0 | 0.0 |
| Brain (whole) | 60.3 | 75.3 | Liver (fetal) | 6.8 | 0.0 |
| Brain (amygdala) | 61.6 | 28.9 | Liver ca. (hepatoblast) HepG2 | 0.0 | 0.0 |
| Brain (cerebellum) | 6.2 | 0.0 | Lung | 19.8 | 10.8 |
| Brain (hippocampus) | 47.3 | 97.9 | Lung (fetal) | 23.2 | 48.6 |
| Brain (substantia nigra) | 28.5 | 82.9 | Lung ca. (small cell) LX-1 | 0.0 | 0.0 |
| Brain (thalamus) | 54.0 | 100.0 | Lung ca. (small cell) NCI-H69 | 0.0 | 0.0 |
| Cerebral Cortex | 0.0 | 27.7 | Lung ca. (s.cell var.) SHP-77 | 0.0 | 0.0 |
| Spinal cord | 77.4 | 28.7 | Lung ca. (large cell) NCI-H460 | 0.0 | 0.0 |
| glio/astro U87-MG | 0.0 | 0.0 | Lung ca. (non-sm. cell) A549 | 0.0 | 0.0 |
| gilo/astro U-118-MG | 6.5 | 10.7 | Lung ca. (non-s.cell) NCI-H23 | 0.0 | 23.7 |
| astrocytoma SW1783 | 8.3 | 0.0 | Lung ca. (non-s.cell) HOP-62 | 0.0 | 10.2 |
| neuro*; met SK-N-AS | 0.0 | 0.0 | Lung ca. (non-s.cl) NCI-H522 | 0.0 | 14.8 |
| astrocytoma SF-539 | 0.0 | 0.0 | Lung ca. (squam.) SW 900 | 0.0 | 26.4 |
| astrocytoma SNB-75 | 7.6 | 19.8 | Lung ca. (squam.) NCI-H596 | 0.0 | 0.0 |
| glioma SNB-19 | 13.2 | 14.4 | Mammary gland | 13.0 | 0.0 |
| glionia U251 | 7.5 | 0.0 | Breast ca.* (pl.ef) MCF-7 | 0.0 | 0.0 |
| glioma SF-295 | 0.0 | 0.0 | Breast ca.* (pl.ef) MDA-MB | 0.0 | 0.0 |
| | | | 231 | | |
| Heart (Fetal) | 0.0 | 0.0 | Breast ca.* (pl.ef) T47D | 0.0 | 0.0 |
| Heart | 0.0 | 0.0 | Breast ca. BT-549 | 2.6 | 27.2 |
| Skeletal muscle (Fetal) | 7.1 | 11.3 | Breast ca. MDA-N | 0.0 | 0.0 |
| Skeletal muscle | 84.7 | 57.0 | Ovary | 24.1 | 0.0 |
| Bone marrow | 0.0 | 0.0 | Ovarian ca. OVCAR-3 | 0.0 | 5.8 |
| Thymus | 19.6 | 0.0 | Ovarian ca. OVCAR-4 | 0.0 | 0.0 |
| Spleen | 0.0 | 13.2 | Ovarian ca. OVCAR-5 | 0.0 | 0.0 |
| Lymph node | 0.0 | 0.0 | Ovarian ca. OVCAR-8 | 0.0 | 0.0 |
| Colorectal | 5.8 | 28.3 | Ovarian ca. IGROV-l | 0.0 | 0.0 |
| Stomach | 0.0 | 0.0 | Ovarian ca. (ascites) SK-OV-3 | 11.3 | 62.9 |
| Small intestine | 7.6 | 24.5 | Uterus | 0.0 | 33.9 |
| Colon ca. SW480 | 0.0 | 0.0 | Placenta | 0.0 | 0.0 |
| Colon ca.* SW620 (SW480 met) | 0.0 | 10.2 | Prostate | 13.3 | 0.0 |
| Colon ca. HT29 | 0.0 | 0.0 | Prostate ca.* (bone met) PC-3 | 0.0 | 0.0 |
| Colon ca. HCT-116 | 19.9 | 0.0 | Testis | 22.2 | 23.0 |
| Colon ca. CaCo-2 | 0.0 | 0.0 | Melanoma Hs688(A).T | 0.0 | 0.0 |
| CC Well to Mod Diff | 16.8 | 9.0 | Melanoma* (met) Hs688(B).T | 0.0 | 0.0 |
| (ODO3866) | | | | | |
| Colon ca. HCC-2998 | 12.5 | 0.0 | Melanoma UACC-62 | 0.0 | 0.0 |
| Gastric ca. (liver met) NCI-N87 | 27.0 | 13.3 | Melanoma M14 | 0.0 | 0.0 |
| Bladder | 28.9 | 12.4 | Melanoma LOX IMVI | 0.0 | 0.0 |
| Trachea | 13.6 | 0.0 | Melanoma* (met) SK-MEL-5 | 0.0 | 0.0 |
| Kidney | 100.0 | 28.5 | Adipose | 14.0 | 9.7 |

TABLE AOD
Panel 2D
Column A - Ret. Exp.(%) Ag2622, Run 163578215
Column B - Ret. Exp.(%) Ag2622, Run 165910584

| Tissue Name | A | B | Tissue Name | A | B |
|---|---|---|---|---|---|
| Normal Colon | 7.3 | 20.4 | Kidney Margin 8120608 | 0.8 | 0.0 |
| CC Well to Mod Diff (ODO3866) | 0.4 | 0.0 | Kidney Cancer 8120613 | 0.0 | 1.4 |
| CC Margin (ODO3866) | 2.7 | 2.7 | Kidney Margin 8120614 | 1.6 | 3.3 |
| CC Gr.2 rectosigmoid (ODO3868) | 3.3 | 0.0 | Kidney Cancer 9010320 | 0.9 | 1.9 |
| CC Margin (ODO3868) | 0.5 | 6.6 | Kidney Margin 9010321 | 5.2 | 12.4 |
| CC Mod Diff (ODO3920) | 0.0 | 0.0 | Normal Uterus | 0.5 | 0.0 |
| CC Margin (ODO3920) | 3.4 | 10.4 | Uterine Cancer 064011 | 4.6 | 9.4 |
| CC Gr.2 ascend colon (ODO3921) | 2.8 | 4.7 | Normal Thyroid | 8.2 | 9.7 |
| CC Margin (ODO3921) | 1.9 | 5.5 | Thyroid Cancer | 1.2 | 10.2 |
| CC from Partial Hepatectomy | 1.2 | 2.0 | Thyroid Cancer A302152 | 2.8 | 2.1 |
| (ODO4309) Mets | | | | | |
| Liver Margin (ODO4309) | 1.0 | 4.5 | Thyroid Margin A302153 | 7.4 | 19.2 |
| Colon mets to lung (OD04451-01) | 0.0 | 0.0 | Normal Breast | 1.9 | 4.7 |
| Lung Margin (OD04451-02) | 0.7 | 1.8 | Breast Cancer | 4.7 | 6.1 |
| Normal Prostate 6546-1 | 30.4 | 27.7 | Breast Cancer (OD04590-01) | 1.3 | 5.2 |
| Prostate Cancer (OD04410) | 7.9 | 15.9 | Breast Cancer Mets | 100.0 | 4.0 |
| | | | (OD04590-03) | | |
| Prostate Margin (OD04410) | 13.9 | 47.3 | Breast Cancer Metastasis | 17.3 | 33.9 |
| Prostate Cancer (OD04720-01) | 5.5 | 8.7 | Breast Cancer | 16.6 | 14.3 |
| Prostate Margin (OD04720-02) | 9.2 | 37.6 | Breast Cancer | 8.2 | 39.8 |
| Normal Lung | 4.6 | 5.3 | Breast Cancer 9100266 | 1.0 | 2.4 |
| Lung Met to Muscle (ODO4286) | 0.7 | 2.6 | Breast Margin 9100265 | 0.5 | 2.0 |
| Muscle Margin (ODO4286) | 5.3 | 19.6 | Breast Cancer A209073 | 0.0 | 17.0 |
| Lung Malignant Cancer | 2.4 | 3.7 | Breast Margin A209073 | 2.3 | 5.3 |
| (OD03126) | | | | | |
| Lung Margin (OD03126) | 4.6 | 7.2 | Normal Liver | 3.1 | 9.3 |
| Lung Cancer (OD04404) | 1.2 | 1.7 | Liver Cancer | 0.3 | 0.0 |
| Lung Margin (OD04404) | 1.5 | 3.6 | Liver Cancer 1025 | 0.8 | 0.0 |
| Lung Cancer (OD04565) | 0.0 | 0.0 | Liver Cancer 1026 | 0.0 | 0.0 |
| Lung Margin (OD04565) | 0.0 | 1.5 | Liver Cancer 6004-T | 0.4 | 4.6 |
| Lung Cancer (OD04237-01) | 3.9 | 17.4 | Liver Tissue 6004-N | 10.0 | 0.0 |
| Lung Margin (OD04237-02) | 0.6 | 6.5 | Liver Cancer 6005-T | 0.5 | 10.0 |
| Ocular Mel Met to Liver | 0.0 | 2.4 | Liver Tissue 6005-N | 0.0 | 0.0 |
| (ODO4310) | | | | | |
| Liver Margin (ODO4310) | 1.4 | 1.3 | Normal Bladder | 2.0 | 10.0 |
| Melanoma Metastasis | 1.0 | 2.5 | Bladder Cancer | 0.6 | 0.0 |
| Lung Margin (OD04321) | 0.9 | 8.5 | Bladder Cancer | 6.9 | 9.0 |
| Normal Kidney | 34.4 | 100.0 | Bladder Cancer (OD04718- | 1.0 | 1.7 |
| | | | 01) | | |
| Kidney Ca, Nuclear grade 2 | 4.8 | 16.5 | Bladder Normal Adjacent | 3.1 | 10.2 |
| (OD04338) | | | (OD04718-03) | | |
| Kidney Margin (OD04338) | 3.9 | 27.4 | Normal Ovary | 1.6 | 4.2 |
| Kidney Ca Nuclear grade 1/2 | 10.81 | 26.4 | Ovarian Cancer | 2.2 | 8.3 |
| (OD04339) | | | | | |
| Kidney Margin (OD04339) | 21.8 | 55.1 | Ovarian Cancer (OD04768- | 0.0 | 0.0 |
| | | | 07) | | |
| Kidney Ca, Clear cell type | 0.8 | 1.8 | Ovary Margin (OD04768-08) | 0.0 | 3.2 |
| (OD04340) | | | | | |
| Kidney Margin (OD04340) | 11.8 | 41.2 | Normal Stomach | 2.1 | 0.0 |
| Kidney Ca, Nuclear grade 3 | 0.0 | 0.0 | Gastric Cancer 9060358 | 0.0 | 0.0 |
| (OD04348) | | | | | |
| Kidney Margin (OD04348) | 17.9 | 28.3 | Stomach Margin 9060359 | 0.0 | 4.5 |
| Kidney Cancer (OD04622-01) | 1.9 | 2.3 | Gastric Cancer 9060395 | 2.0 | 0.0 |
| Kidney Margin (OD04622-03) | 4.6 | 2.4 | Stomach Margin 9060394 | 3.4 | 10.8 |
| Kidney Cancer (OD04450-01) | 2.1 | 6.5 | Gastric Cancer 9060397 | 1.3 | 0.0 |
| Kidney Margin (OD04450-03) | 16.5 | 47.3 | Stomach Margin 9060396 | 0.0 | 2.7 |
| Kidney Cancer 8120607 | 0.6 | 0.0 | Gastric Cancer 064005 | 1.8 | 13.6 |

TABLE AOE
Panel 4D
Column A - Rel. Exp.(%) Ag2622, Run 162554700
Column B - Rel. Exp.(%) Ag2622, Run 165806297

| Tissue Name | A | B | Tissue Name | A | B |
|---|---|---|---|---|---|
| Secondary Th1 act | 0.0 | 0.1 | HUVEC IL-1beta | 0.0 | 0.0 |
| Secondary Th2 act | 0.1 | 0.0 | HUVEC IFN gamma | 0.3 | 0.0 |
| Secondary Tr1 act | 0.0 | 0.0 | HUVEC TNF alpha + IFN gamma | 0.0 | 0.0 |
| Secondary Th1 rest | 0.0 | 0.0 | HUVEC TNF alpha + IL4 | 0.0 | 0.0 |
| Secondary Th2 rest | 0.0 | 0.0 | HUVEC IL-11 | 0.0 | 0.0 |
| Secondary Tr1 rest | 0.0 | 0.0 | Lung Microvascular EC none | 0.4 | 0.1 |
| Primary Th1 act | 0.0 | 0.0 | Lung Microvascular EC TNFalpha + | 0.0 | 0.0 |
| Primary Th2 act | 0.2 | 0.0 | Microvascular Dermal EC none | 0.0 | 0.0 |
| Primary Tr1 act | 0.0 | 0.0 | Microsvasular Dermal EC TNFalpha | 0.1 | 0.0 |
| | | | + IL-1beta | | |
| Primary Th1 rest | 0.0 | 0.0 | Bronchial epithelium TNFalpha + | 0.0 | 0.1 |
| | | | IL1beta | | |
| Primary Th2 rest | 0.1 | 0.0 | Small airway epithelium none | 0.0 | 0.1 |
| Primary Tr1 rest | 0.2 | 0.0 | Small airway epithelium TNFalpha + | 1.1 | 0.4 |
| | | | IL-1beta | | |
| CD45RA CD4 lymphocyte | 0.0 | 0.0 | Coronery artery SMG rest | 0.0 | 0.0 |
| act | | | | | |
| CD45RO CD4 lymphocyte | 0.1 | 0.1 | Coronery artery SMC TNFalpha + IL- | 0.0 | 0.0 |
| | | | 1beta | | |
| CD8 lymphocyte act | 0.0 | 0.0 | Astrocytes rest | 0.0 | 0.0 |
| Secondary CD8 lymphocyte | 0.0 | 0.0 | Astrocytes TNFalpha + IL-1beta | 0.2 | 0.2 |
| rest | | | | | |
| Secondary CD8 lymphocyte | 0.0 | 0.0 | KU-812 (Basophil) rest | 0.0 | 0.0 |
| act | | | | | |
| CD4 lymphocyte none | 0.2 | 0.0 | KU-812 (Basophil) PMA/ionomycin | 0.1 | 0.0 |
| 2ry Th1/Th2/Tr1 anti-CD95 | 0.0 | 0.0 | CCD1106 (Keratinocytes) none | 0.0 | 0.0 |
| CH11 | | | | | |
| LAK cells rest | 0.0 | 0.0 | CCD1106 (Keratinocytes) TNFalpha | 0.0 | 0.1 |
| | | | + IL-1beta | | |
| LAK cells IL-2 | 0.0 | 0.0 | Liver cirrhosis | 0.8 | 0.6 |
| LAK cells IL-2 + IL-12 | 0.0 | 0.0 | Lupus kidney | 0.2 | 0.4 |
| LAK cells IL-2 + IFN gamma | 0.1 | 0.1 | NCI-H292 none | 0.0 | 0.0 |
| LAK cells IL-2 + IL-18 | 0.1 | 0.0 | NCI-H292 IL-4 | 0.0 | 0.0 |
| LAK cells PMA/ionomycin | 0.0 | 0.0 | NCI-H292 IL-9 | 0.0 | 0.1 |
| NK Cells IL-2 rest | 0.1 | 0.0 | NCI-H292 IL-13 | 0.0 | 0.0 |
| Two Way MLR 3 day | 0.0 | 0.0 | NCI-H292 IFN gamma | 0.1 | 0.0 |
| Two Way MLR 5 day | 0.0 | 0.0 | HPAEC none | 0.1 | 0.0 |
| Two Way MLR 7 day | 0.0 | 0.0 | HPAEC TNF alpha + IL-1 beta | 0.0 | 0.0 |
| PBMC rest | 0.0 | 0.0 | Lung fibroblast none | 0.1 | 0.0 |
| PBMC PWM | 0.3 | 0.1 | Lung fibroblast TNF alpha + IL-1 beta | 0.1 | 0.0 |
| PBMC PHA-L | 0.1 | 0.0 | Lung fibroblast IL-4 | 0.0 | 0.1 |
| Ramos (B cell) none | 0.0 | 0.0 | Lung fibroblast IL-9 | 0.4 | 0.1 |
| Ramos (B cell) ionomycin | 0.0 | 0.0 | Lung fibroblast IL-13 | 0.1 | 0.0 |
| B lymphocytes PWM | 0.1 | 0.0 | Lung fibroblast IFN gamma | 0.5 | 0.1 |
| B lymphocytes CD40L and | 0.0 | 0.0 | Dermal fibroblast CCD1070 rest | 0.1 | 0.1 |
| IL-4 | | | | | |
| EOL-1 dbcAMP | 0.0 | 0.0 | Dermal fibroblast CCD1070 TNF | 0.0 | 0.0 |
| | | | alpha | | |
| EOL-1 dbcAMP | 0.2 | 0.1 | Dermal fibroblast CCD1070 IL-1 beta | 0.0 | 0.0 |
| PMA/ionomycin | | | | | |
| Dendritic cells none | 0.0 | 0.0 | Dermal fibroblast IFN gamma | 0.1 | 0.0 |
| Dendritic cells LPS | 0.0 | 0.1 | Dermal fibroblast IL-4 | 0.0 | 0.1 |
| Dendritic cells anti-CD40 | 0.0 | 0.0 | IBD Colitis 2 | 0.0 | 0.0 |
| Monocytes rest | 0.0 | 0.0 | IBD Crohn's | 1.0 | 2.2 |
| Monocytes LPS | 0.0 | 0.0 | Colon | 100.0 | 100.0 |
| Macrophages rest | 0.0 | 0.0 | Lung | 0.2 | 0.0 |
| Macrophages LPS | 0.0 | 0.0 | Thymus | 1.3 | 0.9 |
| HUVEC none | 10.0 | 0.0 | Kidney | 0.5 | 0.0 |
| HUVEC starved | 0.0 | 0.0 | | | |

TABLE AOF
Panel 5 Islet
Column A - Rel. Exp.(%) Ag3953, Run 223846464

| Tissue Name | A | Tissue Name | A |
|---|---|---|---|
| 97457 Patient-02go adipose | 0.0 | 94709 Donor 2 AM - A adipose | 0.0 |
| 97476 Patient-07sk skeletal muscle | 0.0 | 94710 Donor 2 AM - B adipose | 0.0 |
| 97477 Patient-07ut uterus | 0.0 | 94711 Donor 2 AM - C adipose | 0.0 |
| 97478 Patient-07pl placenta | 0.0 | 94712 Donor 2 AD - A adipose | 0.0 |
| 99167 Bayer Patient 1 | 0.0 | 94713 Donor 2 AD - B adipose | 0.0 |
| 97482 Patient-08ut uterus | 0.0 | 94714 Donor 2 AD - C adipose | 0.0 |
| 97483 Patient-08pl placenta | 3.7 | 94742 Donor 3 U - A Mesenchymal | 0.0 |
| | | Stem Cells | |
| 97486 Patient-09sk skeletal muscle | 0.0 | 94743 Donor 3 U - B Mesenchymal | 0.0 |
| | | Stem Cells | |
| 97487 Patient-09ut uterus | 3.8 | 94730 Donor 3 AM - A adipose | 4.4 |
| 97488 Patient-09pl placenta | 0.0 | 94731 Donor 3 AM - B adipose | 0.0 |
| 97492 Patient-10ut uterus | 0.0 | 94732 Donor 3 AM - C adipose | 0.0 |
| 97493 Patient-10pl placenta | 0.0 | 94733 Donor 3 AD - A adipose | 0.0 |
| 97495 Patient-11go adipose | 0.0 | 94734 Donor 3 AD - B adipose | 0.0 |
| 97496 Patient-11sk skeletal muscle | 3.3 | 94735 Donor 3 AD - C adipose | 3.6 |
| 97497 Patient-11ut uterus | 0.0 | 77138 Liver HepG2untreated | 0.0 |
| 97498 Patient-11pl placenta | 4.0 | 73556 Heart Cardiac stromal cells | 0.0 |
| | | (primary) | |
| 97500 Patient-12go adipose | 4.2 | 81735 Small Intestine | 100.0 |
| 97501 Patient-12sk skeletal muscle | 3.5 | 72409 Kidney Proximal Convoluted | 0.0 |
| | | Tubule | |
| 97502 Patient-12ut uterus | 0.0 | 82685 Small intestine Duodenum | 0.0 |
| 97503 Patient-12pl placenta | 0.0 | 90650 Adrenal Adrenocortical | 0.0 |
| | | adenoma | |
| 94721 Donor 2 U - A Mesenchymal | 0.0 | 72410 Kidney HRCE | 0.0 |
| Stem Cells | | | |
| 94722 Donor 2 U - B Mesenchymal | 4.0 | 72411 Kidney HRE | 0.0 |
| Stem Cells | | | |
| 94723 Donor 2 U - C Mesenchymal | 0.0 | 73139 Uterus Uterine smooth muscle | 0.0 |
| Stem Cells | | cells | |

Panel 1.3D Summary: Ag2622 The highest expression of this gene was detected in the brain and the kidney. There was significantly lower expression in the brain cancer cell lines than normal brain samples. This indicates that downregulation of this gene is important in cell proliferation. Hence this expression profile is useful as a diagnostic marker for brain cancer.

This gene was also expressed at low levels in the CNS. Carboxypeptidase is believed to have a role in the degradation of APP and A-beta, the major component of senile plaques in Alzheimer's disease (Matsumoto A, Itoh K, Matsumoto R. A novel carboxypeptidase B that processes native beta-amyloid precursor protein is present in human hippocampus. Eur J Neurosci 2000 January; 12(1):227-38). Therapeutic modulation of this gene, expressed protein and/or use of antibodies or small molecule drugs targeting the gene or gene product are useful in the treatment of Alzheimer's disease.

Panel 2D Summary: Ag2622 This gene was expressed at low levels in the tissues. There was increased expression in normal prostate and kidney compared to the adjacent tumor tissues. There was also increased expression in breast cancer tissues compared to normal breast tissue. Hence, expression of this gene useful as a diagnostic marker in breast, prostate and kidney cancers. Therapeutic modulation of this gene, expressed protein and/or use of antibodies or small molecule drugs targeting the gene or gene product are useful in the treatment of breast, prostate and kidney cancer.

Panel 4D Summary: Ag2622 This gene encoding a putative carboxypeptidase, was expressed in the colon (CT=25-27) and down regulated in colon tissue isolated from Crohn's and colitis patients (CTs>31). The carboxypeptidase family of enzymes has been found in the colon and is associated with colon disease (Sommer H, Schweisfurth H, Schulz M. Serum angiotensin-1-converting enzyme and carboxypeptidase N in Crohn's disease and ulcerative colitis. Enzyme 1986; 35(4):181-8). Therapeutic modulation of this gene, expressed protein and/or use of antibodies or small molecule drugs targeting the gene or gene product are useful in the treatment of IBD.

Panel 5 Islet Summary: Ag3953 This gene, a carboxypeptidase B1 homolog, showed moderate expression in small intestine (CT=32.6). Carboxypeptidase B1 is an endocrine tissue-specific protein and is a useful serum marker for acute pancreatitis and dysfunction of pancreatic transplants (Yamamoto, K. K.; Pousette, A.; Chow, P.; Wilson, H.; El Shami, S.; French, C. K. Isolation of a cDNA encoding a human serum marker for acute pancreatitis: identification of pancreas-specific protein as pancreatic procarboxypeptidase B. J. Biol. Chem. 1992 267: 2575-2581 PMID: 1370825). This class of peptidase has been implicated in hormone maturation and/or degradation of secreted peptides such as insulin, GLP-1, and PACAP. PACAP latter has a major role in metabolic processes. Several carboxypeptidases, like CPE or PC1, have been shown to be involved in development of diabetes and obesity. Therapeutic modulation of this gene, expressed protein and/or use of antibodies or small molecule drugs targeting the gene or gene product are useful in the treatment of diabetes and also, diseases associated with the GI tract and metabolism.

AP. CG55806-04: Factor-IX.

Expression of gene CG55806-04 was assessed using the primer-probe set Ag2613, described in Table APA. Results of the RTQ-PCR runs are shown in Tables APB, APC and APD.

TABLE APA
Probe Name Ag2613

| | | | Start | SEQ ID |
|---|---|---|---|---|
| Primers | Sequences | Length | Position | No |
| Forward | 5′-agccacatgtcttcgatctaca-3′ | 22 | 253 | 1352 |
| Probe | TET-5′-acaacatgttctgtgctggcttccat-3′- | 26 | 211 | 1353 |
| | TAMRA | | | |
| Reverse | 5′-cccactatctccttgacatgaa-3′ | 22 | 175 | 1354 |

TABLE APB
Panel 1.3D
Column A - Rel. Exp.(%) Ag2613, Run 165672326

| Tissue Name | A | Tissue Name | A |
|---|---|---|---|
| Liver adenocarcinoma | 0.0 | Kidney (fetal) | 10.7 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 100.0 |
| Brain (whole) | 0.0 | Liver (fetal) | 76.3 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.3 |
| Spinal cord | 0.0 | Lung ca. (large cell) NCI-H460 | 0.0 |
| glio/astro U87-MG | 0.0 | Lung ca. (non-sm. cell) A549 | 0.0 |
| glio/astro U-118-MG | 0.0 | Lung ca. (non-s.cell) NCI-H23 | 0.0 |
| astrocytoma SW1783 | 0.0 | Lung ca. (non-s.cell) HOP-62 | 0.0 |
| neuro*; met SK-N-AS | 0.0 | Lung ca. (non-s.cl) NCI-H522 | 0.0 |
| astTocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) | 0.2 |
| | | MDA-MB-231 | |
| Heart (Fetal) | 0.0 | Breast ca.* (pl.ef) T47D | 0.0 |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca. (ascites) SK-OV-3 | 0.0 |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone met) PC-3 | 0.0 |
| Colon ca. HCT-116 | 0.0 | Testis | 0.0 |
| Colon ca. CaCo-2 | 0.0 | Melanoma Hs688(A).T | 0.0 |
| CC Well to Mod Diff | 0.0 | Melanoma* (met) Hs688(B).T | 0.0 |
| (OD03866) | | | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) | 0.0 |
| | | SK-MEL-5 | |
| Kidney | 0.0 | Adipose | 0.0 |

TABLE APC
Panel 2.2
Column A - Rel. Exp.(%) Ag2613, Run 175128272

| Tissue Name | A | Tissue Name | A |
|---|---|---|---|
| Normal Colon | 0.4 | Kidney Margin (OD04348) | 0.0 |
| Colon cancer (OD06064) | 0.0 | Kidney malignant cancer | 0.0 |
| | | (OD06204B) | |
| Colon Margin (OD06064) | 0.0 | Kidney normal adjacent tissue | 0.0 |
| | | (OD06204E) | |
| Colon cancer (OD06159) | 0.0 | Kidney Cancer (OD04450-01) | 0.0 |
| Colon Margin (OD06159) | 0.0 | Kidney Margin (OD04450-03) | 0.0 |
| Colon cancer (OD06297-04) | 0.0 | Kidney Cancer 8120613 | 0.0 |
| Colon Margin (OD06297-05) | 0.0 | Kidney Margin 8120614 | 0.0 |
| CC Gr.2 ascend colon (OD03921) | 0.0 | Kidney Cancer 9010320 | 0.0 |
| CC Margin (OD03921) | 0.0 | Kidney Margin 9010321 | 0.0 |
| Colon cancer metastasis (OD06104) | 0.0 | Kidney Cancer 8120607 | 0.0 |
| Lung Margin (OD06104) | 0.0 | Kidney Margin 8120608 | 0.0 |
| Colon mets to lung (OD04451-01) | 0.1 | Normal Uterus | 0.1 |
| Lung Margin (OD04451-02) | 0.0 | Uterine Cancer 064011 | 0.0 |
| Normal Prostate | 0.0 | Normal Thyroid | 0.0 |
| Prostate Cancer (OD04410) | 0.0 | Thyroid Cancer | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283-03) | 0.0 | Normal Breast | 0.0 |
| Ovarian Margin (OD06283-07) | 0.0 | Breast Cancer | 1.5 |
| Ovarian Cancer | 2.1 | Breast Cancer | 0.0 |
| Ovarian cancer (OD06145) | 1.0 | Breast Cancer (OD04590-01) | 0.2 |
| Ovarian Margin (OD06145) | 0.2 | Breast Cancer Mets (OD04590-03) | 0.0 |
| Ovarian cancer (OD06455-03) | 0.0 | Breast Cancer Metastasis | 0.0 |
| Ovarian Margin (OD06455-07) | 0.0 | Breast Cancer | 0.1 |
| Normal Lung | 0.2 | Breast Cancer 9100266 | 0.0 |
| Invasive poor diff. lung adeno | 0.0 | Breast Margin 9100265 | 0.0 |
| (OD04945-01 | | | |
| Lung Margin (OD04945-03) | 0.0 | Breast Cancer A209073 | 0.0 |
| Lung Malignant Cancer (OD03126) | 0.0 | Breast Margin A209073 | 0.0 |
| Lung Margin (OD03126) | 0.0 | Breast cancer (OD06083) | 0.0 |
| Lung Cancer (OD05014A) | 0.0 | Breast cancer node metastasis | 0.0 |
| | | (OD06083) | |
| Lung Margin (OD05014B) | 0.4 | Normal Liver | 100.0 |
| Lung cancer (OD06081) | 0.0 | Liver Cancer 1026 | 1.9 |
| Lung Margin (OD06081) | 0.0 | Liver Cancer 1025 | 60.3 |
| Lung Cancer (OD04237-01) | 0.0 | Liver Cancer 6004-T | 42.6 |
| Lung Margin (OD04237-02) | 0.0 | Liver Tissue 6004-N | 1.4 |
| Ocular Mel Met to Liver (OD04310) | 0.0 | Liver Cancer 6005-T | 3.3 |
| Liver Margin (OD04310) | 51.4 | Liver Tissue 6005-N | 33.7 |
| Melanoma Metastasis | 0.0 | Liver Cancer | 59.0 |
| Lung Margin (OD04321) | 0.0 | Normal Bladder | 0.0 |
| Normal Kidney | 0.0 | Bladder Cancer | 0.0 |
| Kidney Ca, Nuclear grade 2 | 0.0 | Bladder Cancer | 0.0 |
| (OD04338) | | | |
| Kidney Margin (OD04338) | 0.0 | Normal Stomach | 0.1 |
| Kidney Ca Nuclear grade 1/2 | 0.0 | Gastric Cancer 9060397 | 0.0 |
| (OD04339) | | | |
| Kidney Margin (OD04339) | 0.0 | Stomach Margin 9060396 | 0.0 |
| Kidney Ca, Clear cell type (OD04340) | 0.0 | Gastric Cancer 9060395 | 0.2 |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

TABLE APD
Panel 4D
Column A - Rel. Exp.(%) Ag2613, Run 164399517

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 0.0 |
| Primary Tr1 act | 0.0 | Microsyasular Dermal EC TNFalpha + IL- | 0.0 |
| | | 1beta | |
| Primary Th1 rest | 10.0 | Bronchial epithelium TNFalpha + IL1beta | 0.0 |
| Primary Th2 rest | 10.0 | Small airway epithelium none | 0.0 |
| Primary Tr1 rest | 10.0 | Small airway epithelium TNFalpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNFalpha +IL-1beta | 0.0 |
| CD8 lymphocyte act | 0.0 | Astrocytes rest | 0.0 |
| Secondary CDS lymphocyte rest | 0.0 | Astrocytes TNFaIpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 beta | 0.0 |
| PBMC PHA-L | 0.0 | Lung fibroblast IL-4 | 0.0 |
| Ramos (B cell) none | 10.0 | Lung fibroblast IL-9 | 10.0 |
| Ramos (B cell) ionomycin | 10.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1 beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 0.0 |
| Macrophages rest | 0.0 | Lung | 1.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2613 The highest expression of this gene was found in adult liver. Expression of this gene was also detected in fetal liver and fetal kidney samples on this panel (CTs=27-31). This gene encodes a protein that is homologous to factor IX. The secreted form of the protein may be present in the circulatory sysytem and exhibit effects that are unrelated to the site of synthesis. Measurement of the expression level of this gene or expressed protein is useful to test liver function. Measurement of the expression level of this gene or expressed protein can be used to differentiate between liver derived tissue and other tissues. Therapeutic modulation of this gene, expressed protein and/or use of antibodies or small molecule drugs targeting the gene or gene product are effective in increasing the levels of factor IX in the blood, and useful in the treatment of hemophilia and liver related disease.

Panel 2.2 Summary: Ag2613 Expression of this gene was highest in samples derived from liver (CT=27.9), consistant with the results seen in Panel 1.3D. Therefore, expression of this gene is useful to differentiate between normal sections of liver as compared to tumors that are secordary metastases from other sites (such as melanoma).

Panel 4D Summary: Ag2613 This transcript was highly expressed in cirrhotic liver tissue (CT=27.8). Therapeutic modulation of this gene, expressed protein and/or use of antibodies or small molecule drugs targeting the gene or gene product are useful in the treatment of liver cirrhosis.

AQ. CG55828-02: Serine/Threonine-Protein Kinase PAK 5.

Expression of gene CG55828-02 was assessed using the primer-probe set Ag7281, described in Table AQA. Results of the RTQ-PCR runs are shown in Table AQB. Gene CG55828-02 represents full-length physical clone.

TABLE AQA
Probe Name Ag7281

| | | | Start | SEQ ID |
|---|---|---|---|---|
| Primers | Sequences | Length | Position | No |
| Forward | 5′-gtgtcccatgaacagtttcg-3′ | 20 | 894 | 1355 |
| Probe | TET-5′-agcccaggagaccccagggaatactt-3′- | 26 | 846 | 1356 |
| | TAMRA | | | |
| Reverse | 5′-tccccgattttgataaagttg-3′ | 21 | 822 | 1357 |

TABLE AQB
General_screening_panel_v1.7
Column A - Rel. Exp.(%) Ag7281, Run 318350125

| Tissue Name | A | Tissue Name | A |
|---|---|---|---|
| Adipose | 0.0 | Gastric Ca. (liver met.) | 0.0 |
| | | NCI-N87 | |
| HUVEC | 0.0 | Stomach | 0.0 |
| Melanoma* Hs688(A).T | 0.0 | Colon ca. SW-948 | 0.0 |
| Melanoma* Hs688(B).T | 0.0 | Colon ca. SW480 | 0.0 |
| Melanoma (met) SK-MEL-5 | 0.0 | Colon ca. (SW480 met) | 0.0 |
| | | SW620 | |
| Testis | 0.2 | Colon ca. HT29 | 0.0 |
| Prostate ca. (bone met) PC-3 | 0.0 | Colon ca. HCT-116 | 0.0 |
| Prostate ca. DU145 | 0.0 | Colon cancer tissue | 0.0 |
| Prostate pool | 0.2 | Colon ca. SW1116 | 0.0 |
| Uterus pool | 0.0 | Colon ca. Colo-205 | 0.0 |
| Ovarian ca. OVCAR-3 | 0.0 | Colon ca. SW-48 | 0.0 |
| Ovarian ca. (ascites) SK-OV-3 | 0.0 | Colon | 0.0 |
| Ovarian ca. OVCAR-4 | 0.0 | Small Intestine | 0.0 |
| Ovarian ca. OVCAR-5 | 0.0 | Fetal Heart | 0.0 |
| Ovarian ca. IGROV-1 | 0.0 | Heart | 0.0 |
| Ovarian ca. OVCAR-8 | 0.0 | Lymph Node Pool | 0.0 |
| Ovary | 0.3 | Lymph Node pool 2 | 0.0 |
| Breast ca. MCF-7 | 0.0 | Fetal Skeletal Muscle | 0.0 |
| Breast ca. MDA-MB-231 | 0.0 | Skeletal Muscle pool | 0.0 |
| Breast ca. BT 549 | 0.0 | Skeletal Muscle | 0.0 |
| Breast ca. T47D | 0.0 | Spleen | 0.3 |
| 113452 mammary gland | 0.0 | Thymus | 0.1 |
| Trachea | 0.5 | CNS cancer (glio/astro) | 0.0 |
| | | SF-268 | |
| Lung | 0.1 | CNS cancer (glio/astro) | 0.0 |
| | | T98G | |
| Fetal Lung | 0.1 | CNS cancer (neuro; met) | 0.0 |
| | | SK-N-AS | |
| Lung ca. NCI-N417 | 0.1 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 11.2 | CNS cancer (glio) | 0.0 |
| | | SNB-19 | |
| Lung ca. SHP-77 | 3.8 | CNS cancer (glio) | 0.0 |
| | | SF-295 | |
| Lung ca. NCI-H23 | 0.0 | Brain (Amygdala) | 11.7 |
| Lung ca. NCI-H460 | 0.0 | Brain (Cerebellum) | 80.1 |
| Lung ca. HOP-62 | 0.0 | Brain (Fetal) | 100.0 |
| Lung ca. NCI-H522 | 0.0 | Brain (Hippocampus) | 8.1 |
| Lung ca. DMS-114 | 0.0 | Cerebral Cortex pool | 9.7 |
| Liver | 0.0 | Brain (Substantia nigra) | 5.9 |
| Fetal Liver | 0.0 | Brain (Thalamus) | 17.8 |
| Kidney pool | 0.6 | Brain (Whole) | 81.8 |
| Fetal Kidney | 0.0 | Spinal Cord | 0.5 |
| Renal ca. 786-0 | 0.0 | Adrenal Gland | 5.6 |
| Renal ca. A498 | 0.0 | Pituitary Gland | 2.3 |
| Renal ca. ACHN | 0.0 | Salivary Gland | 0.3 |
| Renal ca. UO-31 | 0.0 | Thyroid | 0.1 |
| Renal ca. TK-10 | 0.0 | Pancreatic ca. PANC-1 | 0.0 |
| Bladder | 0.1 | Pancreas pool | 0.4 |

General_screening_panel_v1.7 Summary: Ag7281 The highest expression of this gene was detected in fetal brain (CT=25). This gene was expressed in all central nervous system samples on this panel. Therapeutic modulation of this gene, expressed protein and/or use of antibodies or small molecule drugs targeting the gene or gene product are useful in the treatment of disorders of the central nervous system including Alzheimer's disease, Parkinson's disease, trauma, stroke, epilepsy, pain, multiple sclerosis, schizophrenia, bipolar disorder, depression, anxiety, obsessive compulsive disorder, ataxia, autism, drug and alcohol addiction.

AR. CG55988-04: Organic Cation Transporter OKB1.

Expression of gene CG55988-04 was assessed using the primer-probe set Ag6389, described in Table ARA. Results of the RTQ-PCR runs are shown in Table ARB.

TABLE ARA
Probe Name Ag6389

| | | | Start | SEQ ID |
|---|---|---|---|---|
| Primers | Sequences | Length | Position | No |
| Forward | 5′-tatggttggaaaatttgcc-3′ | 19 | 1341 | 1358 |
| Probe | TET-5′-atacagctgagctgtatccaaccattgt | 30 | 1391 | 1359 |
| | aa-3′-TAMRA | | | |
| Reverse | 5′-ctgtcctaggagctgtggta-3′ | 20 | 1514 | 1360 |

TABLE ARB
General_screening_panel_v1.6
Column A - Rel. Exp.(%) Ag6389, Run 277246969

| Tissue Name | A | Tissue Name | A |
|---|---|---|---|
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) SW620 | 0.0 |
| SCC-4 | | | |
| Testis Pool | 100.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 1.6 | Colon ca. SW-48 | 0.0 |
| Ovarian ca. OVCAR-4 | 0.0 | Colon Pool | 5.5 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 2.5 |
| Breast ca. MDA-MB-231 | 0.0 | Lymph Node Pool | 1.5 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 2.5 | CNS cancer (glio/astro) U87-MG | 0.0 |
| Lung | 0.0 | CNS cancer (glio/astro) U-118-MG | 0.0 |
| Fetal Lung | 2.7 | CNS cancer (neuro; met) SK-N-AS | 0.0 |
| Lung ca. NCI-N417 | 2.5 | CNS cancer (astro) SF-539 | 0.0 |
| Lung ca. LX-1 | 0.0 | CNS cancer (astro) SNB-75 | 0.0 |
| Lung Ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 2.3 |
| Lung ca. NCI-H522 | 0.0 | Brain (Substantia nigra) Pool | 0.0 |
| Liver | 0.0 | Brain (Thalamus) Pool | 0.0 |
| Fetal Liver | 20.3 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 5.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

General_screening_panel_v1.6 Summary: Ag6389 Low expression of this gene was detected in testis. Modulation of this gene, encoded protein and/or use of antibodies or small molecule drug targeting this gene or gene product will be useful in the treatment testis related disorders including fertility and hypogonadism.

AS. CG56071-01: Mixed Lineage Kinase 2-Like.

Expression of gene CG56071-01 was assessed using the primer-probe sets Ag2872 and Ag4847, described in Tables ASA and ASB. Results of the RTQ-PCR runs are shown in Tables ASC, ASD, ASE, ASF, ASG, ASH and ASI.

TABLE ASA
Probe Name Ag2872

| | | | Start | SEQ ID |
|---|---|---|---|---|
| Primers | Sequences | Length | Position | No |
| Forward | 5′-tcagccagaccatagagaatgt-3′ | 22 | 506 | 1361 |
| Probe | TET-5′-atgctgaagcaccccaacatcattg-3′- | 25 | 553 | 1362 |
| | TAMRA | | | |
| Reverse | 5′-ctccttcagacatacccctctt-3′ | 22 | 582 | 1363 |

TABLE ASB
Probe Name Ag4847

| | | | Start | SEQ ID |
|---|---|---|---|---|
| Primers | Sequences | Length | Position | No |
| Forward | 5′-catagagaatgttcgccaagag-3′ | 22 | 516 | 1364 |
| Probe | TET-5′-atgctgaagcaccccaacatcattg-3′- | 25 | 553 | 1365 |
| | TAMRA | | | |
| Reverse | 5′-ctccttcagacatacccctctt-3′ | 22 | 582 | 1366 |

TABLE ASC
General_screening_panel_v1.5
Column A - Rel. Exp.(%) Ag4847, Run 228796410

| Tissue Name | A | Tissue Name | A |
|---|---|---|---|
| Adipose | 1.0 | Renal ca. TK-10 | 13.9 |
| Melanoma* Hs688(A).T | 0.1 | Bladder | 5.8 |
| Melanoma* Hs688(B).T | 0.0 | Gastric ca. (liver met.) NCI-N87 | 21.5 |
| Melanoma* M14 | 5.4 | Gastric ca. KATO III | 14.7 |
| Melanoma* LOXIMVI | 1.6 | Colon ca. SW-948 | 4.3 |
| Melanoma* SK-MEL-5 | 9.2 | Colon ca. SW480 | 16.2 |
| Squamous cell carcinoma | 28.9 | Colon ca.* (SW480 met) SW620 | 5.4 |
| SCC-4 | | | |
| Testis Pool | 2.8 | Colon ca. HT29 | 4.8 |
| Prostate ca.* (bone met) | 7.2 | Colon ca. HCT-116 | 13.4 |
| PC-3 | | | |
| Prostate Pool | 1.5 | Colon ca. CaCo-2 | 13.6 |
| Placenta | 5.3 | Colon cancer tissue | 3.0 |
| Uterus Pool | 1.0 | Colon ca. SW1116 | 2.3 |
| Ovarian ca. OVCAR-3 | 10.1 | Colon ca. Colo-205 | 2.7 |
| Ovarian ca. SK-OV-3 | 23.5 | Colon ca. SW-48 | 2.1 |
| Ovarian ca. OVCAR-4 | 7.1 | Colon Pool | 0.6 |
| Ovarian ca. OVCAR-5 | 24.0 | Small Intestine Pool | 1.3 |
| Ovarian ca. IGROV-1 | 29.5 | Stomach Pool | 1.3 |
| Ovarian ca. OVCAR-8 | 3.7 | Bone Marrow Pool | 0.9 |
| Ovary | 1.0 | Fetal Heart | 0.9 |
| Breast ca. MCF-7 | 10.5 | Heart Pool | 0.3 |
| Breast ca. MDA-MB-231 | 10.9 | Lymph Node Pool | 0.9 |
| Breast ca. BT 549 | 0.4 | Fetal Skeletal Muscle | 0.1 |
| Breast ca. T47D | 11.7 | Skeletal Muscle Pool | 2.9 |
| Breast ca. MDA-N | 1.6 | Spleen Pool | 2.4 |
| Breast Pool | 1.1 | Thymus Pool | 2.5 |
| Trachea | 3.9 | CNS cancer (glio/astro) | 13.3 |
| | | U87-MG | |
| Lung | 0.3 | CNS cancer (glio/astro) | 0.5 |
| | | U-118-MG | |
| Fetal Lung | 4.2 | CNS cancer (neuro; met) | 3.7 |
| | | SK-N-AS | |
| Lung ca. NCI-N417 | 2.1 | CNS cancer (astro) SF-539 | 0.8 |
| Lung ca. LX-1 | 8.8 | CNS cancer (astro) SNB-75 | 0.2 |
| Lung ca. NCI-H146 | 5.6 | CNS cancer (glio) SNB-19 | 4.0 |
| Lung ca. SHP-77 | 13.1 | CNS cancer (glio) SF-295 | 2.8 |
| Lung ca. A549 | 13.4 | Brain (Amygdala) Pool | 8.2 |
| Lung ca. NCI-H526 | 4.0 | Brain (cerebellum) | 100.0 |
| Lung ca. NCI-H23 | 7.3 | Brain (fetal) | 15.9 |
| Lung ca. NCI-H460 | 1.1 | Brain (Hippocampus) Pool | 8.1 |
| Lung ca. HOP-62 | 4.7 | Cerebral Cortex Pool | 18.3 |
| Lung ca. NCI-H522 | 7.1 | Brain (Substantia nigra) Pool | 24.0 |
| Liver | 0.4 | Brain (Thalamus) Pool | 13.7 |
| Fetal Liver | 1.2 | Brain (whole) | 18.8 |
| Liver ca. HepG2 | 6.0 | Spinal Cord Pool | 3.5 |
| Kidney Pool | 0.5 | Adrenal Gland | 2.6 |
| Fetal Kidney | 2.6 | Pituitary gland Pool | 2.3 |
| Renal ca. 786-0 | 10.8 | Salivary Gland | 1.8 |
| Renal ca. A498 | 9.5 | Thyroid (female) | 1.0 |
| Renal ca. ACHN | 11.0 | Pancreatic ca. CAPAN2 | 19.2 |
| Renal ca. UO-31 | 15.9 | Pancreas Pool | 2.5 |

TABLE ASD
Panel 1.3D
Column A - Rel. Exp.(%) Ag2872, Run 161971644
Column B - Rel. Exp.(%) Ag2872, Run 165721686
Column C - Rel. Exp.(%) Ag2872, Run 166006455

| Tissue Name | A | B | C | Tissue Name | A | B | C |
|---|---|---|---|---|---|---|---|
| Liver adenocarcinoma | 16.7 | 22.5 | 21.8 | Kidney (fetal) | 1.9 | 2.9 | 1.1 |
| Pancreas | 0.3 | 4.3 | 5.4 | Renal ca. 786-0 | 2.8 | 17.6 | 15.0 |
| Pancreatic ca. CAPAN 2 | 5.5 | 45.4 | 44.8 | Renal ca. A498 | 4.5 | 36.1 | 12.7 |
| Adrenal gland | 0.5 | 2.2 | 0.3 | Renal ca. RXF 393 | 13.5 | 85.9 | 77.9 |
| Thyroid | 0.9 | 1.7 | 1.6 | Renal ca. ACHN | 6.4 | 17.8 | 17.8 |
| Salivary gland | 0.5 | 3.7 | 5.4 | Renal ca. UO-31 | 5.0 | 27.0 | 19.3 |
| Pituitaiy gland | 1.7 | 11.4 | 4.3 | Renal ca. TK-10 | 2.3 | 7.9 | 10.0 |
| Brain (fetal) | 2.9 | 24.5 | 22.1 | Liver | 0.2 | 0.0 | 1.0 |
| Brain (whole) | 6.1 | 66.4 | 79.6 | Liver (fetal) | 0.4 | 0.8 | 0.7 |
| Brain (amygdala) | 6.4 | 49.7 | 41.8 | Liver ca. (hepatoblast) | 4.3 | 14.7 | 18.6 |
| | | | | HepG2 | | | |
| Brain (cerebellum) | 13.1 | 82.9 | 100.0 | Lung | 0.5 | 6.0 | 9.0 |
| Brain (hippocampus) | 10.2 | 32.8 | 29.3 | Lung (fetal) | 0.5 | 4.3 | 0.7 |
| Brain (substantia nigra) | 2.0 | 14.5 | 12.2 | Lung ca. (small cell) LX- | 3.4 | 16.0 | 11.6 |
| | | | | 1 | | | |
| Brain (thalamus) | 7.4 | 42.6 | 63.3 | Lung ca. (small cell) NCI | 3.6 | 26.1 | 26.4 |
| | | | | H69 | | | |
| Cerebral Cortex | 100.0 | 84.1 | 90.1 | Lung ca. (s.cell var.) | 11.1 | 25.0 | 13.8 |
| | | | | SHP-77 | | | |
| Spinal cord | 0.9 | 3.1 | 3.6 | Lung ca. (large cell)NCI- | 0.3 | 4.3 | 43.2 |
| | | | | H460 | | | |
| glio/astro U87-MG | 13.2 | 14.5 | 15.7 | Lung ca. (non-sm. cell) | 3.9 | 8.0 | 9.9 |
| | | | | A549 | | | |
| glio/astro U-118-MG | 0.0 | 0.0 | 0.0 | Lung ca. (non-s.cell) | 3.0 | 6.8 | 3.6 |
| | | | | NCI-H23 | | | |
| astrocytoma SW1783 | 7.3 | 7.9 | 10.3 | Lung ca. (non-s.cell) | 2.0 | 8.7 | 6.4 |
| | | | | HOP-62 | | | |
| neuro*; met SK-N-AS | 1.2 | 5.9 | 2.2 | Lung ca. (non-s.cl) NCI- | 2.6 | 5.0 | 3.1 |
| | | | | H522 | | | |
| astrocytoma SF-539 | 1.0 | 3.7 | 6.0 | Lung ca. (squam.) SW | 15.6 | 58.2 | 94.0 |
| | | | | 900 | | | |
| astrocytoma SNB-75 | 9.7 | 100.0 | 21.3 | Lung ca. (squam.) NCI- | 2.5 | 16.5 | 19.3 |
| | | | | H596 | | | |
| glioma SNB-19 | 2.5 | 6.4 | 6.7 | Mammary gland | 0.7 | 6.2 | 2.0 |
| glioma U251 | 2.8 | 22.2 | 8.9 | Breast ca.* (pl.ef) MCF-7 | 11.3 | 21.9 | 16.7 |
| glioma SF-295 | 1.2 | 3.7 | 6.3 | Breast ca.* (pl.ef) MDA- | 4.1 | 32.3 | 6.7 |
| | | | | MB-231 | | | |
| Heart (Fetal) | 0.5 | 0.6 | 1.7 | Breast ca.* (pl.ef) T47D | 3.5 | 17.0 | 14.4 |
| Heart | 0.1 | 1.1 | 0.0 | Breast ca. BT-549 | 0.3 | 4.7 | 0.8 |
| Skeletal muscle (Fetal) | 0.8 | 0.7 | 0.3 | Breast ca. MDA-N | 1.0 | 0.9 | 3.3 |
| Skeletal muscle | 1.0 | 4.4 | 2.5 | Ovary | 2.3 | 1.4 | 0.0 |
| Bone marrow | 0.4 | 1.4 | 0.9 | Ovarian ca. OVCAR-3 | 3.9 | 19.1 | 10.2 |
| Thymus | 2.5 | 0.7 | 2.3 | Ovarian ca. OVCAR-4 | 1.6 | 25.2 | 22.1 |
| Spleen | 0.7 | 6.0 | 3.3 | Ovarian ca. OVCAR-5 | 6.3 | 27.7 | 40.6 |
| Lymph node | 1.4 | 8.7 | 3.2 | Ovarian ca. OVCAR-8 | 3.3 | 14.0 | 6.2 |
| Colorectal | 7.5 | 6.7 | 6.8 | Ovarian ca. IGROV-1 | 1.4 | 4.6 | 11.2 |
| Stomach | 0.9 | 8.3 | 2.8 | Ovarian ca. (ascites) SK- | 6.4 | 46.7 | 39.2 |
| | | | | OV-3 | | | |
| Small intestine | 0.8 | 3.2 | 2.1 | Uterus | 0.1 | 1.1 | 0.0 |
| Colon ca. SW480 | 2.5 | 6.4 | 18.9 | Placenta | 2.2 | 6.8 | 9.7 |
| Colon ca.* SW620 (SW480 | 3.1 | 13.8 | 9.9 | Prostate | 0.5 | 2.6 | 2.8 |
| met) | | | | | | | |
| Colon ca. HT29 | 3.2 | 3.1 | 4.2 | Prostate ca.* (bone met) | 4.4 | 39.2 | 16.6 |
| | | | | PC-3 | | | |
| Colon ca. HCT-116 | 2.9 | 15.0 | 4.8 | Testis | 2.9 | 7.3 | 3.7 |
| Colon ca. CaCo-2 | 9.0 | 11.7 | 9.9 | Melanoma Hs688(A).T | 0.1 | 0.0 | 0.0 |
| CC Well to Mod Duff | 4.0 | 6.9 | 4.8 | Melanoma* (met) | 0.0 | 0.0 | 0.0 |
| (ODO3866) | | | | Hs688(B).T | | | |
| Colon ca. HCC-2998 | 3.1 | 12.6 | 11.1 | Melanoma UACC-62 | 1.6 | 6.3 | 9.3 |
| Gastric ca. (liver met) NCI- | 11.7 | 54.3 | 23.2 | Melanoma M14 | 0.9 | 11.9 | 2.1 |
| N87 | | | | | | | |
| Bladder | 7.3 | 14.7 | 6.6 | Melanoma LOX IMVI | 0.4 | 0.0 | 0.4 |
| Trachea | 2.8 | 5.9 | 2.0 | Melanoma* (met) SK- | 2.1 | 8.4 | 6.0 |
| | | | | MEL-S | | | |
| Kidney | 2.0 | 5.8 | 0.4 | Adipose | 0.7 | 2.4 | 2.4 |

TABLE ASE
Panel 2.2
Column A - Rel. Exp.(%) Ag2872, Run 175149214

| Tissue Name | A | Tissue Name | A |
|---|---|---|---|
| Normal Colon | 17.4 | Kidney Margin (OD04348) | 100.0 |
| Colon cancer (OD06064) | 26.6 | Kidney malignant cancer | 14.1 |
| | | (OD06204B) | |
| Colon Margin (OD06064) | 16.2 | Kidney normal adjacent tissue | 8.5 |
| | | (OD06204E) | |
| Colon cancer (OD06159) | 4.4 | Kidney Cancer (OD04450-01) | 29.1 |
| Colon Margin (OD06159) | 11.5 | Kidney Margin (OD04450-03) | 21.3 |
| Colon cancer (OD06297-04) | 1.1 | Kidney Cancer 8120613 | 2.3 |
| Colon Margin (OD06297-05) | 19.6 | Kidney Margin 8120614 | 16.7 |
| CC Gr.2 ascend colon (ODO3921) | 4.4 | Kidney Cancer 9010320 | 0.0 |
| CC Margin (ODO3921) | 3.5 | Kidney Margin 9010321 | 10.4 |
| Colon cancer metastasis (ODO6104) | 0.0 | Kidney Cancer 8120607 | 16.3 |
| Lung Margin (OD06104) | 6.6 | Kidney Margin 8120608 | 7.5 |
| Colon mets to lung (OD04451-01) | 33.0 | Normal Uterus | 0.0 |
| Lung Margin (OD04451-02) | 10.8 | Uterine Cancer 064011 | 2.2 |
| Normal Prostate | 7.3 | Normal Thyroid | 0.0 |
| Prostate Cancer (OD04410) | 8.0 | Thyroid Cancer | 5.8 |
| Prostate Margin (OD04410) | 5.8 | Thyroid Cancer A302152 | 20.0 |
| Normal Ovary | 2.4 | Thyroid Margin A302153 | 3.4 |
| Ovarian cancer (OD06283-03) | 4.2 | Normal Breast | 21.6 |
| Ovarian Margin (OD06283-07) | 8.2 | Breast Cancer | 8.3 |
| Ovarian Cancer | 12.2 | Breast Cancer | 22.5 |
| Ovarian cancer (OD06145) | 4.3 | Breast Cancer (OD04590-01) | 41.2 |
| Ovarian Margin (OD06145) | 8.7 | Breast Cancer Mets (OD04590-03) | 26.1 |
| Ovarian cancer (OD06455-03) | 20.2 | Breast Cancer Metastasis | 46.0 |
| Ovarian Margin (OD06455-07) | 0.0 | Breast Cancer | 15.8 |
| Normal Lung | 7.6 | Breast Cancer 9100266 | 9.6 |
| Invasive poor diff. lung adeno | 27.5 | Breast Margin 9100265 | 1.7 |
| ODO4945-01 | | | |
| Lung Margin (ODO4945-03) | 14.1 | Breast Cancer A209073 | 9.9 |
| Lung Malignant Cancer (OD03126) | 16.0 | Breast Margin A209073 | 17.2 |
| Lung Margin (OD03126) | 4.3 | Breast cancer (OD06083) | 50.3 |
| Lung Cancer (OD05014A) | 7.5 | Breast cancer node metastasis | 42.0 |
| | | (0D06083) | |
| Lung Margin (OD05014B) | 16.2 | Normal Liver | 12.2 |
| Lung cancer (OD06081) | 23.8 | Liver Cancer 1026 | 3.2 |
| Lung Margin (OD06081) | 12.3 | Liver Cancer 1025 | 10.4 |
| Lung Cancer (OD04237-01) | 9.9 | Liver Cancer 6004-T | 0.3 |
| Lung Margin (OD04237-02) | 25.5 | Liver Tissue 6004-N | 6.8 |
| Ocular Mel Met to Liver (ODO4310) | 4.3 | Liver Cancer 6005-T | 13.6 |
| Liver Margin (ODO4310) | 3.7 | Liver Tissue 6005-N | 8.8 |
| Melanoma Metastasis | 5.8 | Liver Cancer | 21.2 |
| Lung Margin (OD04321) | 15.1 | Normal Bladder | 17.4 |
| Normal Kidney | 1.8 | Bladder Cancer | 4.6 |
| Kidney Ca, Nuclear grade 2 | 41.8 | Bladder Cancer | 22.4 |
| (OD04338) | | | |
| Kidney Margin (OD04338) | 10.4 | Normal Stomach | 27.7 |
| Kidney Ca Nuclear grade 1/2 | 52.1 | Gastric Cancer 9060397 | 3.6 |
| (OD04339) | | | |
| Kidney Margin (OD04339) | 15.3 | Stomach Margin 9060396 | 14.2 |
| Kidney Ca, Clear cell type (OD04340) | 0.0 | Gastric Cancer 9060395 | 5.1 |
| Kidney Margin (OD04340) | 16.8 | Stomach Margin 9060394 | 10.2 |
| Kidney Ca, Nuclear grade 3 | 0.0 | Gastric Cancer 064005 | 12.1 |
| (OD04348) | | | |

TABLE ASF
Panel 2D
Column A - Rel. Exp.(%) Ag2872, Run 161971795

| Tissue Name | A | Tissue Name | A |
|---|---|---|---|
| Normal Colon | 30.8 | Kidney Margin 8120608 | 2.9 |
| CC Well to Mod Diff (ODO3866) | 13.0 | Kidney Cancer 8120613 | 7.6 |
| CC Margin (ODO3866) | 5.9 | Kidney Margin 8120614 | 15.3 |
| CC Gr.2 rectosigmoid (ODO3868) | 16.2 | Kidney Cancer 9010320 | 10.9 |
| CC Margin (ODO3868) | 3.4 | Kidney Margin 9010321 | 23.0 |
| CC Mod Diff(ODO3920) | 16.3 | Normal Uterus | 0.0 |
| CC Margin (ODO3920) | 10.9 | Uterine Cancer 064011 | 23.5 |
| CC Gr.2 ascend colon (ODO3921) | 20.4 | Normal Thyroid | 4.0 |
| CC Margin (ODO3921) | 4.8 | Thyroid Cancer | 14.5 |
| CC from Partial Hepatectomy | 21.6 | Thyroid Cancer A302152 | 15.0 |
| (ODO4309) Mets | | | |
| Liver Margin (ODO4309) | 6.2 | Thyroid Margin A302153 | 11.7 |
| Colon mets to lung (OD04451-01) | 24.0 | Normal Breast | 21.3 |
| Lung Margin (OD04451-02) | 9.5 | Breast Cancer | 25.9 |
| Normal Prostate 6546-1 | 1.8 | Breast Cancer (OD04590-01) | 42.3 |
| Prostate Cancer (OD04410) | 19.1 | Breast Cancer Mets (OD04590-03) | 39.2 |
| Prostate Margin (OD04410) | 15.2 | Breast Cancer Metastasis | 40.9 |
| Prostate Cancer (OD04720-01) | 17.2 | Breast Cancer | 15.9 |
| Prostate Margin (OD04720-02) | 18.3 | Breast Cancer | 24.8 |
| Normal Lung | 31.0 | Breast Cancer 9100266 | 23.8 |
| Lung Met to Muscle (ODO4286) | 12.1 | Breast Margin 9100265 | 7.9 |
| Muscle Margin (ODO4286) | 4.6 | Breast Cancer A209073 | 23.2 |
| Lung Malignant Cancer (OD03126) | 35.4 | Breast Margin A209073 | 17.2 |
| Lung Margin (OD03126) | 24.8 | Normal Liver | 4.2 |
| Lung Cancer (OD04404) | 43.2 | Liver Cancer | 10.1 |
| Lung Margin (OD04404) | 14.2 | Liver Cancer 1025 | 3.3 |
| Lung Cancer (OD04565) | 26.6 | Liver Cancer 1026 | 5.3 |
| Lung Margin (OD04565) | 8.1 | Liver Cancer 6004-T | 4.6 |
| Lung Cancer (OD04237-01) | 25.2 | Liver Tissue 6004-N | 8.5 |
| Lung Margin (OD04237-02) | 16.6 | Liver Cancer 6005-T | 5.6 |
| Ocular Mel Met to Liver (ODO4310) | 7.0 | Liver Tissue 6005-N | 1.6 |
| Liver Margin (ODO4310) | 9.2 | Normal Bladder | 28.7 |
| Melanoma Metastasis | 8.0 | Bladder Cancer | 15.1 |
| Lung Margin (OD04321) | 32.1 | Bladder Cancer | 27.4 |
| Normal Kidney | 32.5 | Bladder Cancer (OD04718-01) | 32.1 |
| Kidney Ca, Nuclear grade 2 (OD04338) | 30.8 | Bladder Normal Adjacent | 0.9 |
| | | (OD04718-03) | |
| Kidney Margin (OD04338) | 32.8 | Normal Ovary | 2.9 |
| Kidney Ca Nuclear grade 1/2 (OD04339) | 31.6 | Ovarian Cancer | 17.1 |
| Kidney Margin (OD04339) | 23.3 | Ovarian Cancer (OD04768-07) | 100.0 |
| Kidney Ca, Clear cell type (OD04340) | 5.5 | Ovary Margin (OD04768-08) | 1.5 |
| Kidney Margin (OD04340) | 29.5 | Normal Stomach | 15.8 |
| Kidney Ca, Nuclear grade 3 (OD04348) | 0.5 | Gastric Cancer 9060358 | 2.5 |
| Kidney Margin (OD04348) | 27.9 | Stomach Margin 9060359 | 7.5 |
| Kidney Cancer (OD04622-01) | 6.0 | Gastric Cancer 9060395 | 8.8 |
| Kidney Margin (OD04622-03) | 4.5 | Stomach Margin 9060394 | 14.4 |
| Kidney Cancer (OD04450-01) | 16.5 | Gastric Cancer 9060397 | 33.0 |
| Kidney Margin (OD04450-03) | 19.2 | Stomach Margin 9060396 | 7.9 |
| Kidney Cancer 8120607 | 11.5 | Gastric Cancer 064005 | 23.0 |

TABLE ASG
Panel 3D
Column A - Rel. Exp.(%) Ag2872, Run 164543502
Column B - Rel. Exp.(%) Ag2872, Run 164828587

| Tissue Name | A | B | Tissue Name | A | B |
|---|---|---|---|---|---|
| 94905 Daoy | 2.5 | 1.9 | 94954 Ca Ski Cervical | 8.2 | 9.7 |
| Medulloblastoma/Cerebellum | | | epidermoid carcinoma | | |
| | | | (metastasis | | |
| 94906 TE671 | 1.5 | 2.0 | 94955 ES-2 Ovarian clear cell | 0.6 | 0.5 |
| Medulloblastom/Cerebellum | | | carcinoma | | |
| 94907 D283 Med | 6.3 | 8.8 | 94957 Ramos Stimulated with | 3.0 | 3.3 |
| Medulloblastoma/Cerebellum | | | PMA/ionomycin 6h | | |
| 94908 PFSK-1 Primitive | 1.3 | 1.6 | 94958 Ramos Stimulated with | 3.0 | 3.8 |
| Neuroectodermal/Cerebellum | | | PMA/ionomycin 14h | | |
| 94909 XF-498 CNS | 0.3 | 0.4 | 94962 MEG-01 Chronic | 0.4 | 0.8 |
| | | | myelogenous leukemia | | |
| | | | (megokaryoblast) | | |
| 94910 SNB-78 CNS/glioma | 0.0 | 0.0 | 94963 Raji Burkitt's | 1.2 | 1.1 |
| | | | lymphoma | | |
| 94911 SF-268 CNS/glioblastoma | 0.7 | 0.9 | 94964 Daudi Burkitt's | 2.2 | 2.4 |
| | | | lymphoma | | |
| 94912 T98G Glioblastoma | 0.7 | 1.2 | 94965 U266 B-cell | 1.5 | 1.1 |
| | | | plasmacytoma/myeloma | | |
| 96776 SK-N-SH Neuroblastoma | 1.2 | 2.0 | 94968 CA46 Burkitt's | 1.4 | 0.8 |
| (metastasis) | | | lymphoma | | |
| 94913 SF-295 CNS/glioblastoma | 0.4 | 0.6 | 94970 RL non-Hodgkin's B- | 0.7 | 0.9 |
| | | | cell lymphoma | | |
| 94914 Cerebellum | 7.0 | 10.4 | 94972 JM1 pre-B-cell | 1.2 | 1.7 |
| | | | lymphoma/leukemia | | |
| 96777 Cerebellum | 7.9 | 12.1 | 94973 Jurkat T cell leukemia | 1.7 | 1.8 |
| 94916 NCI-H292 | 20.9 | 25.5 | 94974 TF-1 Erythroleukemia | 0.2 | 0.2 |
| Mucoepidermoid lung carcinoma | | | | | |
| 94917 DMS-114 Small cell lung | 1.6 | 1.7 | 94975 HUT 78 T-cell | 0.9 | 1.6 |
| cancer | | | lymphoma | | |
| 94918 DMS-79 Small cell lung | 100.0 | 100.0 | 94977 U937 Histiocytic | 0.6 | 1.4 |
| cancer/neuroendocrine | | | lymphoma | | |
| 94919 NCI-H146 Small cell lung | 8.5 | 8.9 | 94980 KU-812 Myelogenous | 0.1 | 0.2 |
| cancer/neuroendocrine | | | leukemia | | |
| 94920 NCI-H526 Small cell lung | 9.7 | 13.6 | 769-P- Clear cell renal | 1.7 | 2.2 |
| cancer/neuroendocrine | | | carcinoma | | |
| 94921 NCI-N417 Small cell lung | 2.5 | 2.9 | 94983 Caki-2 Clear cell renal | 1.8 | 2.6 |
| cancer/neuroendocrine carcinoma | | | | | |
| 94923 NCI-H82 Small cell lung | 1.5 | 1.7 | 94984 SW 839 Clear cell renal | 2.1 | 2.2 |
| cancer/neuroendocrine | | | carcinoma | | |
| 94924 NCI-H157 Squamous cell | 7.4 | 10.1 | 94986 G401 Wilms' tumor | 0.8 | 1.6 |
| lung cancer (metastasis) | | | | | |
| 94925 NCI-H1155 Large cell | 8.7 | 10.8 | 94987 Hs766T Pancreatic | 2.8 | 3.1 |
| lung cancer/neuroendocrine | | | carcinoma (LN metastasis) | | |
| 94926 NCI-H1299 Large cell | 5.1 | 5.1 | 94988 CAPAN-1 Pancreatic | 3.3 | 3.4 |
| lung cancer/neuroendocrine | | | adenocarcinoma (liver | | |
| | | | metastasis) | | |
| 94927 NCI-H727 Lung carcinoid | 6.2 | 6.7 | 94989 SU86.86 Pancreatic | 5.7 | 7.4 |
| | | | carcinoma (liver metastasis) | | |
| 94928 NCI-UMC-11 Lung | 17.8 | 15.4 | 94990 BxPC-3 Pancreatic | 7.1 | 10.0 |
| carcinoid | | | adenocarcinoma | | |
| 94929 LX-1 Small cell lung | 5.1 | 4.3 | 94991 HPAC Pancreatic | 4.4 | 3.2 |
| cancer | | | adenocarcinoma | | |
| 94930 Colo-205 Colon cancer | 3.5 | 4.5 | 94992 MIA PaCa-2 Pancreatic | 0.8 | 1.6 |
| | | | carcinoma | | |
| 94931 KM12 Colon cancer | 5.2 | 6.0 | 94993 CFPAC-1 Pancreatic | 30.4 | 28.5 |
| | | | ductal adenocarcinoma | | |
| 94932 KM20L2 Colon cancer | 1.3 | 0.9 | 94994 PANC-1 Pancreatic | 3.8 | 3.7 |
| | | | epithelioid ductal carcinoma | | |
| 94933 NCI-H716 Colon cancer | 10.8 | 13.9 | 94996 T24 Bladder carcinma | 2.5 | 2.4 |
| | | | (transitional cell | | |
| 94935 SW-48 Colon | 1.6 | 1.3 | 5637- Bladder carcinoma | 5.7 | 6.0 |
| adenocarcinoma | | | | | |
| 94936 SW1116 Colon | 2.9 | 2.9 | 94998 HT-1197 Bladder | 6.0 | 6.3 |
| adenocarcinoma | | | carcinoma | | |
| 94937 LS 174T Colon | 6.2 | 6.9 | 94999 UM-UC-3 Bladder | 0.8 | 0.6 |
| adenocarcinoma | | | carcinma (transitional cell) | | |
| 94938 SW-948 Colon | 0.7 | 0.6 | 95000 A204 | | |
| adenocarcinoma | | | Rhabdomyosarcoma | 0.9 | 1.2 |
| 94939 SW-480 Colon | 2.2 | 0.2 | 95001 HT-1080 Fibrosarcoma | 8.2 | 11.8 |
| adenocarcinoma | | | | | |
| 94940 NCI-SNU-5 Gastric | 2.8 | 3.4 | 95002 MG-63 Osteosarcoma | 0.0 | 0.0 |
| carcinoma | | | (bone) | | |
| KATO III- Gastric carcinoma | 3.3 | 6.3 | 95003 SK-LMS-1 | 1.7 | 1.8 |
| | | | Leiomyosarcoma (vulva) | | |
| 94943 NCI-SNU-16 Gastric | 3.9 | 3.8 | 95004 SJRH30 | 1.4 | 0.7 |
| carcinoma | | | Rhabdomyosarcoma (met to | | |
| | | | bone marrow) | | |
| 94944 NCI-SNU-1 Gastric | 6.0 | 8.0 | 95005 A431 Epidermoid | 4.1 | 5.6 |
| carcinoma | | | carcinoma | | |
| 94946 RF-1 Gastric | 1.6 | 2.3 | 95007 WM266-4 Melanoma | 2.0 | 2.1 |
| adenocarcinoma | | | | | |
| 94947 RF-48 Gastric | 2.3 | 1.8 | DU 145- Prostate carcinoma | 0.3 | 0.1 |
| adenocarcinoma | | | (brain metastasis) | | |
| 96778 MKN-45 Gastric | 7.0 | 7.6 | 95012 MDA-MB-468 Breast | 4.6 | 7.5 |
| carcinoma | | | adenocarcinoma | | |
| 94949 NCI-N87 Gastric | 4.4 | 4.8 | SCC-4- Squamous cell | 0.8 | 0.4 |
| carcinoma | | | carcinoma of tongue | | |
| 94951 OVCAR-5 Ovarian | 0.9 | 1.4 | SCC-9- Squamous cell | 0.7 | 0.5 |
| carcinoma | | | carcinoma of tongue | | |
| 94952 RL95-2 Uterine carcinoma | 2.3 | 3.0 | SGC-15- Squamous cell | 0.4 | 0.3 |
| | | | carcinoma of tongue | | |
| 94953 HelaS3 Cervical | 1.4 | 2.2 | 95017 CAL 27 Squamous cell | 3.1 | 3.4 |
| adenocarcinoma | | | carcinoma of tongue | | |

TABLE ASH
Panel 4.1D
Column A - Rel. Exp.(%) Ag4847, Run 223335762

| Tissue Name | A | Tissue Name | A |
|---|---|---|---|
| Secondary Th1 act | 12.2 | HUVEC IL-1beta | 0.0 |
| Secondary Th2 act | 14.8 | HUVEC IFN gamma | 0.0 |
| Secondary Tr1 act | 9.5 | HUVEC TNF alpha + IFN gamma | 0.0 |
| Secondary Th1 rest | 2.6 | HUVEC TNF alpha + IL4 | 0.0 |
| Secondary Th2 rest | 8.7 | HUVEC IL-11 | 0.0 |
| Secondary Tr1 rest | 9.9 | Lung Microvascular EC none | 0.0 |
| Primary Th1 act | 2.1 | Lung Microvascular EC TNFalpha + IL- | 0.0 |
| | | 1beta | |
| Primary Th2 act | 14.2 | Microvascular Dermal EC none | 0.0 |
| Primary Tr1 act | 2.8 | Microsvasular Dermal EC TNFalpha + IL- | 0.0 |
| | | 1beta | |
| Primary Th1 rest | 2.7 | Bronchial epithelium TNFalpha + IL1beta | 51.8 |
| Primary Th2 rest | 3.6 | Small airway epithelium none | 28.7 |
| Primary Tr1 rest | 19.1 | Small airway epithelium TNFalpha + IL- | 52.5 |
| | | 1beta | |
| CD45RA CD4 lymphocyte act | 5.3 | Coronery artery SMC rest | 0.0 |
| CD45RO CD4 lymphocyte act | 9.1 | Coronery artery SMC TNFalpha + IL-1beta | 0.1 |
| CD8 lymphocyte act | 3.9 | Astrocytes rest | 16.7 |
| Secondary CD8 lymphocyte rest | 6.6 | Astrocytes TNFalpha + IL-1beta | 9.7 |
| Secondary CD8 lymphocyte act | 2.4 | KU-812 (Basophil) rest | 0.3 |
| CD4 lymphocyte none | 0.5 | KU-812 (Basophil) PMA/ionomycin | 0.2 |
| 2ry Th1/Th2/Tr1 anti-CD95 | 7.0 | CCD1106 (Keratinocytes) none | 68.8 |
| CH11 | | | |
| LAK cells rest | 1.9 | CCD1106 (Keratinocytes) TNFalpha + IL- | 29.5 |
| | | 1beta | |
| LAK cells IL-2 | 6.3 | Liver cirrhosis | 4.1 |
| LAK cells IL-2 + IL-12 | 6.2 | NCI-H292 none | 27.7 |
| LAK cells IL-2 + IFN gamma | 4.0 | NCI-H292 IL-4 | 87.1 |
| LAK cells IL-2 + IL-18 | 3.5 | NCI-H292 IL-9 | 81.2 |
| LAK cells PMA/ionomycin | 3.3 | NCI-H292 IL-13 | 74.7 |
| NK Cells IL-2 rest | 5.1 | NCI-H292 IFN gamma | 35.4 |
| Two Way MLR 3 day | 1.6 | HPAEC none | 0.0 |
| Two Way MLR 5 day | 1.3 | HPAEC TNF alpha + IL-1 beta | 0.0 |
| Two Way MLR 7 day | 1.6 | Lung fibroblast none | 0.4 |
| PBMC rest | 3.8 | Lung fibroblast TNF alpha + IL-1 beta | 0.5 |
| PBMC PWM | 5.6 | Lung fibroblast IL-4 | 0.0 |
| PBMC PHA-L | 13.0 | Lung fibroblast IL-9 | 0.0 |
| Ramos (B cell) none | 15.1 | Lung fibroblast IL-13 | 0.0 |
| Ramos (B cell) ionomycin | 15.5 | Lung fibroblast IFN gamma | 0.0 |
| B lymphocytes PWM | 9.2 | Dermal fibroblast CCD1070 rest | 0.0 |
| B lymphocytes CD40L and IL-4 | 42.0 | Dermal fibroblast CCD1070 TNF alpha | 6.2 |
| EOL-1 dbcAMP | 0.0 | Dermal fibroblast CCD1070 IL-1 beta | 0.0 |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.7 |
| Macrophages rest | 0.0 | Lung | 8.8 |
| Macrophages LPS | 0.0 | Thymus | 32.5 |
| HUVEC none | 0.0 | Kidney | 100.0 |
| HUVEC starved | 0.0 | | |

TABLE ASI
Panel 5 Islet
Column A - Rel. Exp.(%) Ag2872, Run 237228677

| Tissue Name | A | Tissue Name | A |
|---|---|---|---|
| 97457 Patient-02go adipose | 5.1 | 94709 Donor 2 AM - A adipose | 1.8 |
| 97476 Patient-07sk skeletal muscle | 2.7 | 94710 Donor 2 AM - B adipose | 0.0 |
| 97477 Patient-07ut uterus | 0.0 | 94711 Donor 2 AM - C adipose | 0.0 |
| 97478 Patient-07pl placenta | 39.5 | 94712 Donor 2 AD - A adipose | 0.0 |
| 99167 Bayer Patient 1 | 97.9 | 94713 Donor 2 AD - B adipose | 0.0 |
| 97482 Patient-08ut uterus | 0.0 | 94714 Donor 2 AD - C adipose | 0.0 |
| 97483 Patient-08pl placenta | 21.9 | 94742 Donor 3 U - A Mesenchymal | 0.0 |
| | | Stem Cells | |
| 97486 Patient-09sk skeletal muscle | 2.5 | 94743 Donor 3 U - B Mesenchymal | 0.0 |
| | | Stem Cells | |
| 97487 Patient-09ut uterus | 0.0 | 94730 Donor 3 AM - A adipose | 0.0 |
| 97488 Patient-09pl placenta | 19.3 | 94731 Donor 3 AM - B adipose | 0.0 |
| 97492 Patient-10ut uterus | 1.9 | 94732 Donor 3 AM - C adipose | 0.0 |
| 97493 Patient-10pl placenta | 100.0 | 94733 Donor 3 AD - A adipose | 0.0 |
| 97495 Patient-11go adipose | 4.0 | 94734 Donor 3 AD - B adipose | 0.0 |
| 97496 Patient-11sk skeletal muscle | 3.2 | 94735 Donor 3 AD - C adipose | 0.0 |
| 97497 Patient-11ut uterus | 1.7 | 77138 Liver HepG2untreated | 46.0 |
| 97498 Patient-11pl placenta | 23.8 | 73556 Heart Cardiac stromal cells | 0.0 |
| | | (primary) | |
| 97500 Patient-12go adipose | 1.6 | 81735 Small Intestine | 13.5 |
| 97501 Patient-12sk skeletal muscle | 4.0 | 72409 Kidney Proximal Convoluted | 17.1 |
| | | Tubule | |
| 97502 Patient-12ut uterus | 0.0 | 82685 Small intestine Duodenum | 1.4 |
| 97503 Patient-12pl placenta | 20.3 | 90650 Adrenal Adrenocortical | 1.6 |
| | | adenoma | |
| 94721 Donor 2 U - A Mesenchymal | 0.0 | 72410 Kidney HRCE | 90.1 |
| Stem Cells | | | |
| 94722 Donor 2 U - B Mesenchymal | 0.0 | 72411 Kidney HRE | 48.6 |
| Stem Cells | | | |
| 94723 Donor 2 U - C Mesenchymal | 0.0 | 73139 Uterus Uterine smooth muscle | 2.9 |
| Stem Cells | | cells | |

General_screening_panel_v1.5 Summary: Ag4847 Expression of this gene was highest in the cerebellum (CT=25.4). This gene was also expressed at more moderate levels in other central nervous system tissues, including amygdala, hippocampus, cerebral cortex, substantia nigra, thalamus and spinal cord (CTs=27-30). This gene encodes a protein with homology to mixed lineage kinase 2. Mixed lineage kinase 2 is a mammalian protein kinase that activates stress-activated protein kinases/c-jun N-terminal kinases (SAPK/JNKs) through direct phosphorylation of their upstream activator, SEK1/JNKK. MAP kinase signaling pathways are important mediators of cellular responses to a wide variety of stimuli. Signals pass along these pathways via kinase cascades in which three protein kinases are sequentially phosphorylated and activated, initiating a range of cellular programs including cellular proliferation, endocrine, immune and inflammatory responses, and apoptosis. Mixed lineage kinases have been implicated in neuronal apoptosis (Xu Z, Maroney A C, Dobrzanski P, Kukekov N V, Greene L A. The MLK family mediates c-Jun N-terminal kinase activation in neuronal apoptosis. Mol Cell Biol 2001 July; 21(14):4713-24). Therapeutic modulation of this gene, expressed protein and/or use of antibodies or small molecule drugs targeting the gene or gene product are useful in slowing neuronal apoptosis in the treatment of neurodegenerative diseases such as Alzheimer's, Huntington's and Parkinson's diseases.

This gene also showed significant expression in cell lines drived from ovarian cancers. Therapeutic modulation of this gene, expressed protein and/or use of antibodies or small molecule drugs targeting the gene or gene product are useful in the treatment of ovarian cancer.

This gene was expressed at low to moderate levels in endocrine and metabolic tissues including adipose, adrenal gland, liver, pancreas, pituitary gland, skeletal muscle and thyroid. Therapeutic modulation of this gene, expressed protein and/or use of antibodies or small molecule drugs targeting the gene or gene product are useful in the treatment of endocrine/metabolic-related disorders, such as obesity and diabetes.

Panel 1.3D Summary: Ag2872 This gene showed highest expression in samples derived from brain tissue, either normal tissue or cell lines derived from malignant brain tissue. Please see panel General_Screening_V1.5 for a discussion of this gene in the central nervous system.

There was substantial expression of this gene in a number of cancer cell lines, including ovarian cancer, breast cancer and renal cancer cell lines. Therapeutic modulation of this gene, expressed protein and/or use of antibodies or small molecule drugs targeting the gene or gene product are useful in the treatment of ovarian, breast or renal cancer.

There was limited expression of this gene in endocrine/metabolic related tissues. Low expression of this gene was seen in adipose, pancreas, reproductive tissues (testes and ovaries) and skeletal muscle. Therapeutic modulation of this gene, expressed protein and/or use of antibodies or small molecule drugs targeting the gene or gene product are useful in the treatment of different endocrine/metabolic diseases, such as diabetes and obesity.

Panel 2.2 Summary: Ag2872 Expression of this gene was highest in a sample derived from normal kidney tissue adjacent to a kidney cancer (CT=31.2). In addition, there was substantial expression of this gene in samples derived from a cluster of breast cancers. Therapeutic modulation of this gene, expressed protein and/or use of antibodies or small molecule drugs targeting the gene or gene product are useful in the treatment of breast cancer.

Panel 2D Summary: Ag2872 Expression of this gene was highest in a sample derived from an ovarian cancer (CT=28.4). Thus, expression of this gene can be used to distinguish ovarian cancer tissue from the other tissues in the panel. There was substantial expression of this gene in samples derived from a cluster of breast cancers and well as a small but appreciable difference in expression between a set of colon cancers and their respective normal adjacent tissues. Therapeutic modulation of this gene, expressed protein and/or use of antibodies or small molecule drugs targeting the gene or gene product are useful in the treatment of breast cancer, ovarian cancer or colon cancer.

Panel 3D Summary: Ag2872 This gene showed highest expression in a sample derived from a small cell lung cancer derived cell line (CT=26.1). There was substantial expression of this gene in two other lung cancer derived cell lines and a pancreatic cancer derived cell line. Therapeutic modulation of this gene, expressed protein and/or use of antibodies or small molecule drugs targeting the gene or gene product are useful in the treatment of lung cancer.

Panel 4.1D Summary: Ag4847 Expression of this gene was highest in kidney (CT=28.3). This gene was also highly expressed in small airway epithelium treated with TNF-a and IL-1b, and to a lower extent in the same non treated tissue and also in the mucoepidermoid cell line H292 upon treatment with the Th2 cytokines IL-4 and Il-9, cytokines that are responsible for increasing mucus production in this cell line. Expression of this gene was up-regulated in bronchial epithelium upon TNF-a and IL-1 treatment. Moderate expression of this gene was also seen in activated B cells. This gene encodes for a mixed lineage kinase 2 (MLK2) like molecule which was reported to activate JNK pathway (Hirai S, Noda K, Moriguchi T, Nishida E, Yamashita A, Deyama T, Fukuyama K, Ohno S. Differential activation of two JNK activators, MKK7 and SEK1, by MKN28-derived nonreceptor serine/threonine kinase/mixed lineage kinase 2. J Biol Chem 1998 Mar. 27; 273(13):7406-12). Activation of this pathway has been associated to many inflammatory reactions in many cell types. Il-1b which is produced during airway inflammation, has been shown to regulate JNK pathway, for example (Hallsworth M P, Moir L M, Lai D, Hirst S J. Inhibitors of mitogen-activated protein kinases differentially regulate eosinophil-activating cytokine release from human airway smooth muscle. Am J Respir Crit Care Med 2001 Aug. 15; 164(4):688-97). The role of Il-4 and IL-13 in airway remodeling appears also to use JNK pathway (Hashimoto S, Gon Y, Takeshita I, Maruoka S, Horie T. IL-4 and IL-13 induce myofibroblastic phenotype of human lung fibroblasts through c-Jun NH2-terminal kinase-dependent pathway. J Allergy Clin Immunol 2001 June; 107(6):1001-8). Finally, JNK is required for the production of metalloproteinases (Han Z, Boyle D L, Chang L, Bennett B, Karin M, Yang L, Manning A M, Firestein G S. c-Jun N-terminal kinase is required for metalloproteinase expression and joint destruction in inflammatory arthritis. J Clin Invest 2001 July; 108(1):73-81), molecules that play an important role in inflammatory disesease such as rheumatoid arthritis, asthma, and inflammatory bowel disease (IBD). Therapeutic modulation of this gene, expressed protein and/or use of antibodies or small molecule drugs targeting the gene or gene product are useful in the treatment of inflammatory diseseas such as in chronic obstructive pulmonary disease, asthma, emphysema and also rheumatoid arthritis/osteoarthritis, IBD and psoriasis.

Panel 5 Islet Summary: Ag2872 This gene was expressed at low to moderate levels in pancreatic islet cells and placenta in panel 5I. Please refer to General_screening_panel_v1.5 for a synopsis of the the potential function of this MLK2-like gene in endocrine and metabolic disorders.

AT. CG56142-01 and CG56142-04: Prostasin.

Expression of gene CG56142-01 and CG56142-04 were assessed using the primer-probe sets Ag2888 and Ag4095, respectively, described in Table ATA and ATB. Results of the RTQ-PCR runs are shown in Tables ATC, ATD, ATE, ATF, ATG and ATH.

TABLE ATA
Probe Name Ag2888

| | | | Start | SEQ ID |
|---|---|---|---|---|
| Primers | Sequences | Length | Position | No |
| Forward | 5′-aatgagaggggtttcctgtct-3′ | 21 | 18 | 1368 |
| Probe | TET-5′-caggtcctgctccttctggtgctg-3′- | 24 | 40 | 1369 |
| | TAMRA | | | |
| Reverse | 5′-caacgatccgactggacat-3′ | 19 | 82 | 1370 |

TABLE ATB
Probe Name Ag4095

| | | | Start | SEQ ID |
|---|---|---|---|---|
| Primers | Sequences | Length | Position | No |
| Forward | 5′-aatgagaggggtttcctgtct-3′ | 21 | 13 | 1371 |
| Probe | TET-5′-caggtcctgctccttctggtgctg-3′- | 24 | 35 | 1372 |
| | TAMRA | | | |
| Reverse | 5′-gcagacttccttccctgagt-3′ | 20 | 71 | 1373 |

TABLE ATC
General_screening_panel_v1.4
Column A - Rel. Exp.(%) Ag4095, Run 219575329

| Tissue Name | A | Tissue Name | A |
|---|---|---|---|
| Adipose | 0.0 | Renal ca. TK-10 | 0.2 |
| Melanoma* Hs688(A).T | 0.0 | Bladder | 0.0 |
| Melanoma* Hs688(B).T | 0.0 | Gastric ca. (liver met.) NCI-N87 | 0.1 |
| Melanoma* M14 | 0.0 | Gastric ca. KATO III | 1.3 |
| Melanoma* LOXIMVI | 0.0 | Colon ca. SW-948 | 0.2 |
| Melanoma* SK-MEL-5 | 0.0 | Colon ca. SW480 | 2.9 |
| Squamous cell carcinoma | 0.0 | Colon ca.* (SW480 met) SW620 | 0.0 |
| SCC-4 | | | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 100.0 |
| Uterus Pool | 0.0 | Colon ca. SW1116 | 0.1 |
| Ovarian ca. OVCAR-3 | 0.0 | Colon ca. Colo-205 | 0.0 |
| Ovarian ca. SK-OV-3 | 0.0 | Colon ca. SW-48 | 0.1 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 7.5 | Stomach Pool | 0.0 |
| Ovarian ca. OVCAR-8 | 2.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.2 | Lymph Node Pool | 0.0 |
| Breast ca. BT 549 | 0.0 | Fetal Skeletal Muscle | 0.0 |
| Breast ca. T47D | 0.5 | Skeletal Muscle Pool | 0.0 |
| Breast ca. MDA-N | 0.2 | Spleen Pool | 0.0 |
| Breast Pool | 0.0 | Thymus Pool | 0.0 |
| Trachea | 0.0 | CNS cancer (glio/astro) U87-MG | 0.0 |
| Lung | 1.2 | CNS cancer (glio/astro) U-118-MG | 0.0 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.2 |
| Lung Ca. NCI-H146 | 0.0 | CNS cancer (glio) SNB-19 | 6.3 |
| Lung ca. SHP-77 | 0.7 | CNS cancer (glio) SF-295 | 0.5 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.1 |
| Liver | 0.0 | Brain (Thalamus) Pool | 0.0 |
| Fetal Liver | 0.1 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.1 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE ATD
General_screening_panel_v1.5
Column A - Rel. Exp.(%) Ag2888, Run 258495050

| Tissue Name | A | Tissue Name | A |
|---|---|---|---|
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI-N87 | 0.0 |
| Melanoma* M14 | 0.2 | Gastric ca. KATO III | 1.3 |
| Melanoma* LOXIMVI | 0.0 | Colon ca. SW-948 | 0.5 |
| Melanoma* SK-MEL-5 | 0.0 | Colon ca. SW480 | 2.7 |
| Squamous cell carcinoma | 0.0 | Colon ca.* (SW480 met) SW620 | 0.0 |
| SCC-4 | | | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 100.0 |
| Uterus Pool | 0.0 | Colon ca. SW1116 | 0.0 |
| Ovarian ca. OVCAR-3 | 0.0 | Colon ca. Colo-205 | 0.2 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 6.6 | Stomach Pool | 0.0 |
| Ovarian ca. OVCAR-8 | 1.4 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.3 | Spleen Pool | 0.0 |
| Breast Pool | 0.0 | Thymus Pool | 0.0 |
| Trachea | 0.2 | CNS cancer (glio/astro) U87-MG | 0.0 |
| Lung | 0.0 | CNS cancer (glio/astro) U-118-MG | 0.3 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung Ca. NCI-H146 | 0.0 | CNS cancer (glio) SNB-19 | 11.8 |
| Lung ca. SHP-77 | 0.0 | CNS cancer (glio) SF-295 | 0.1 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE ATE
Panel 2D
Column A - Rel. Exp.(%) Ag2888, Run 160897960

| Tissue Name | A | Tissue Name | A |
|---|---|---|---|
| Normal Colon | 0.0 | Kidney Margin 8120608 | 0.0 |
| CC Well to Mod Diff (ODO3866) | 100.0 | Kidney Cancer 8120613 | 0.0 |
| CC Margin (ODO3866) | 1.1 | Kidney Margin 8120614 | 0.0 |
| CC Gr.2 rectosigmoid (ODO3868) | 0.0 | Kidney Cancer 9010320 | 0.5 |
| CC Margin (ODO3868) | 0.0 | Kidney Margin 9010321 | 0.0 |
| CC Mod Diff(ODO3920) | 3.5 | Normal Uterus | 0.0 |
| CC Margin (ODO3920) | 0.0 | Uterine Cancer 064011 | 0.0 |
| CC Gr.2 ascend colon (ODO3921) | 1.2 | Normal Thyroid | 0.0 |
| CC Margin (ODO3921) | 0.1 | Thyroid Cancer | 0.0 |
| CC from Partial Hepatectomy | 14.2 | Thyroid Cancer A302152 | 0.0 |
| (ODO4309) Mets | | | |
| Liver Margin (ODO4309) | 0.2 | Thyroid Margin A302153 | 0.0 |
| Colon mets to lung (OD04451-01) | 3.5 | Normal Breast | 0.0 |
| Lung Margin (OD04451-02) | 0.0 | Breast Cancer | 0.0 |
| Normal Prostate 6546-1 | 0.0 | Breast Cancer (OD04590-01) | 0.0 |
| Prostate Cancer (OD04410) | 0.0 | Breast Cancer Mets (OD04590-03) | 0.0 |
| Prostate Margin (OD04410) | 0.0 | Breast Cancer Metastasis | 0.0 |
| Prostate Cancer (OD04720-01) | 0.0 | Breast Cancer | 0.0 |
| Prostate Margin (OD04720-02) | 0.0 | Breast Cancer | 0.0 |
| Normal Lung | 0.0 | Breast Cancer 9100266 | 0.0 |
| Lung Met to Muscle (ODO4286) | 0.0 | Breast Margin 9100265 | 0.0 |
| Muscle Margin (ODO4286) | 0.0 | Breast Cancer A209073 | 0.0 |
| Lung Malignant Cancer (OD03126) | 0.2 | Breast Margin A209073 | 0.0 |
| Lung Margin (OD03126) | 0.0 | Normal Liver | 0.0 |
| Lung Cancer (OD04404) | 0.0 | Liver Cancer | 0.0 |
| Lung Margin (OD04404) | 0.0 | Liver Cancer 1025 | 0.2 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.2 | Liver Tissue 6004-N | 0.0 |
| Lung Margin (OD04237-02) | 0.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 0.0 |
| Melanoma Metastasis | 0.7 | Bladder Cancer | 0.0 |
| Lung Margin (OD04321) | 0.0 | Bladder Cancer | 0.0 |
| Normal Kidney | 0.0 | Bladder Cancer (OD04718-01) | 0.3 |
| Kidney Ca, Nuclear grade 2 (OD04338) | 0.0 | Bladder Normal Adjacent | 0.0 |
| | | (OD04718-03) | |
| Kidney Margin (OD04338) | 0.0 | Normal Ovary | 0.4 |
| Kidney Ca Nuclear grade 1/2 (OD04339) | 0.0 | Ovarian Cancer | 0.0 |
| Kidney Margin (OD04339) | 0.0 | Ovarian Cancer (OD04768-07) | 0.1 |
| Kidney Ca, Clear cell type (OD04340) | 0.0 | Ovary Margin (OD04768-08) | 0.0 |
| Kidney Margin (OD04340) | 0.0 | Normal Stomach | 0.7 |
| Kidney Ca, Nuclear grade 3 (OD04348) | 0.3 | Gastric Cancer 9060358 | 0.0 |
| Kidney Margin (OD04348) | 0.0 | Stomach Margin 9060359 | 0.5 |
| Kidney Cancer (OD04622-01) | 0.0 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 17.6 |
| Kidney Margin (OD04450-03) | 0.0 | Stomach Margin 9060396 | 0.0 |
| Kidney Cancer 8120607 | 0.0 | Gastric Cancer 064005 | 0.0 |

TABLE ATF
Panel 3D
Column A - Rel. Exp.(%) Ag2888, Run 164629839

| Tissue Name | A | Tissue Name | A |
|---|---|---|---|
| 94905 Daoy | 0.0 | 94954 Ca Ski Cervical epidermoid | 0.0 |
| Medulloblastoma/Cerebellum | | carcinoma (metastasis | |
| 94906 TE671 | 0.0 | 94955 ES-2 Ovarian clear cell | 0.0 |
| Medulloblastom/Cerebellum | | carcinoma | |
| 94907 D283 Med | 0.0 | 94957 Ramos Stimulated with | 0.0 |
| Medulloblastoma/Cerebellum | | PMA/ionomycin 6 h | |
| 94908 PFSK-1 Primitive | 0.0 | 94958 Ramos Stimulated with | 0.0 |
| Neuroectodermal/Cerebellum | | PMA/ionomycin 14 h | |
| 94909 XF-498 CNS | 0.0 | 94962 MEG-01 Chronic | 0.0 |
| | | myelogenous leukemia | |
| | | (megokaryoblast) | |
| 94910 SNB-78 CNS/glioma | 0.0 | 94963 Raji Burkitt's lymphoma | 0.0 |
| 94911 SF-268 CNS/glioblastoma | 0.0 | 94964 Daudi Burkitt's lymphoma | 0.0 |
| 94912 T98G Glioblastoma | 0.0 | 94965 U266 B-cell | 0.0 |
| | | plasmacytoma/myeloma | |
| 96776 SK-N-SH Neuroblastoma | 0.0 | 94968 CA46 Burkitt's lymphoma | 0.0 |
| (metastasis) | | | |
| 94913 SF-295 CNS/glioblastoma | 0.0 | 94970 RL non-Hodgkin's B-cell | 0.0 |
| | | lymphoma | |
| 94914 Cerebellum | 0.0 | 94972 JM1 pre-B-cell | 0.0 |
| | | lymphoma/leukemia | |
| 96777 Cerebellum | 0.0 | 94973 Jurkat T cell leukemia | 0.0 |
| 94916 NCI-H292 Mucoepidermoid | 0.0 | 94974 TF-l Erythroleukemia | 0.0 |
| lung carcinoma | | | |
| 94917 DMS-114 Small cell lung | 0.0 | 94975 HUT 78 T-cell lymphoma | 0.0 |
| cancer | | | |
| 94918 DMS-79 Small cell lung | 0.0 | 94977 U937 Histiocytic lymphoma | 0.0 |
| cancer/neuroendocrine | | | |
| 94919 NCI-H146 Small cell lung | 0.0 | 94980 KU-812 Myelogenous | 0.0 |
| cancer/neuroendocrine | | leukemia | |
| 94920 NCI-H526 Small cell lung | 0.0 | 769-P-Clear cell renal carcinoma | 0.0 |
| cancer/neuroendocrine | | | |
| 94921 NCI-N417 Small cell lung | 0.0 | 94983 Caki-2 Clear cell renal | 0.0 |
| cancer/neuroendocrine | | carcinoma | |
| 94923 NCI-H82 Small cell lung | 0.0 | 94984 SW 839 Clear cell renal | 0.0 |
| cancer/neuroendocrine | | carcinoma | |
| 94924 NCI-H157 Squamous cell lung | 0.0 | 94986 G401 Wilms'tumor | 0.0 |
| cancer (metastasis) | | | |
| 94925 NCI-H1155 Large cell lung | 0.0 | 94987 Hs766T Pancreatic carcinoma | 77.9 |
| cancer/neuroendocrine | | (LN metastasis) | |
| 94926 NCI-H1299 Large cell lung | 0.0 | 94988 CAPAN-1 Pancreatic | |
| cancer/neuroendocrine | | adenocarcinoma (liver metastasis) | 1.6 |
| 94927 NCI-H727 Lung carcinoid | 0.0 | 94989 SU86.86 Pancreatic carcinoma | 2.5 |
| | | (liver metastasis) | |
| 94928 NCI-UMC-11 Lung carcinoid | 0.0 | 94990 BxPC-3 Pancreatic | 0.0 |
| | | adenocarcinoma | |
| 94929 LX-1 Small cell lung cancer | 0.0 | 94991 HPAC Pancreatic | 0.0 |
| | | adenocarcinoma | |
| 94930 Colo-205 Colon cancer | 0.0 | 94992 MIA PaCa-2 Pancreatic | 0.0 |
| | | carcinoma | |
| 94931 KM12 Colon cancer | 0.0 | 94993 CFPAC-1 Pancreatic ductal | 0.0 |
| | | adenocarcinoma | |
| 94932 KM20L2 Colon cancer | 0.0 | 94994 PANC-1 Pancreatic epithelioid | 0.0 |
| | | ductal carcinoma | |
| 94933 NCI-H716 Colon cancer | 0.0 | 94996 T24 Bladder carcinma | 0.0 |
| | | (transitional cell | |
| 94935 SW-48 Colon adenocarcinoma | 0.0 | 5637-Bladder carcinoma | 0.0 |
| 94936 SW1116 Colon | 0.0 | 94998 HT-1197 Bladder carcinoma | 0.0 |
| adenocarcinoma | | | |
| 94937 LS 174T Colon | 0.0 | 94999 UM-UC-3 Bladder carcinma | 0.0 |
| adenocarcinoma | | (transitional cell) | |
| 94938 SW-948 Colon | 0.0 | 95000 A204 Rhabdomyosarcoma | 0.0 |
| adenocarcinoma | | | |
| 94939 SW-480 Colon | 0.0 | 95001 HT-1080 Fibrosarcoma | 0.0 |
| adenocarcinoma | | | |
| 94940 NCI-SNU-5 Gastric carcinoma | 100.0 | 95002 MG-63 Osteosarcoma (bone) | 0.0 |
| KATO III-Gastric carcinoma | 0.0 | 95003 SK-LMS-1 Leiomyosarcoma | 0.0 |
| | | (vulva) | |
| 94943 NCI-SNU-16 Gastric | 0.0 | 95004 SJRH30 Rhabdomyosarcoma | 0.0 |
| carcinoma | | (met to bone marrow) | |
| 94944 NCI-SNU-1 Gastric carcinoma | 0.0 | 95005 A431 Epidermoid carcinoma | 0.0 |
| 94946 RF-1 Gastric adenocarcinoma | 0.0 | 95007 WM266-4 Melanoma | 0.0 |
| 94947 RF-48 Gastric adenocarcinoma | 0.0 | DU 145-Prostate carcinoma (brain | 0.0 |
| | | metastasis) | |
| 96778 MKN-45 Gastric carcinoma | 0.0 | 95012 MDA-MB-468 Breast | 0.0 |
| | | adenocarcinoma | |
| 94949 NCI-N87 Gastric carcinoma | 27.9 | SCC-4-Squamous cell carcinoma of | 0.0 |
| | | tongue | |
| 94951 OVCAR-5 Ovarian carcinoma | 0.0 | SCC-9-Squamous cell carcinoma of | 0.0 |
| | | tongue | |
| 94952 RL95-2 Uterine carcinoma | 0.0 | SCC-15-Squamous cell carcinoma of | 0.0 |
| | | tongue | |
| 94953 HelaS3 Cervical | 0.0 | 95017 CAL 27 Squamous cell | 0.0 |
| adenocarcinoma | | carcinoma of tongue | |

TABLE ATG
Panel 4.1D
Column A - Rel. Exp.(%) Ag4095, Run 172383943
Column B - Rel. Exp.(%) Ag4095, Run 268719443

| Tissue Name | A | B | Tissue Name | A | B |
|---|---|---|---|---|---|
| Secondary Th1 act | 0.0 | 0.0 | HUVEC IL-1beta | 0.0 | 0.0 |
| Secondary Th2 act | 0.0 | 0.0 | HUVEC IFN gamma | 0.0 | 0.0 |
| Secondary Tr1 act | 0.0 | 0.0 | HUVEC TNF alpha + IFN gamma | 0.0 | 0.0 |
| Secondary Th1 rest | 0.0 | 0.0 | HUVEC TNF alpha + IL4 | 0.0 | 0.0 |
| Secondary Th2 rest | 0.0 | 0.0 | HUVEC IL-11 | 0.0 | 0.0 |
| Secondary Tr1 rest | 0.0 | 0.0 | Lung Microvascular EC none | 0.0 | 0.0 |
| Primary Th1 act | 0.0 | 0.0 | Lung Microvascular EC TNFalpha + | 0.0 | 0.0 |
| | | | IL-1beta | | |
| Primary Th2 act | 0.0 | 0.0 | Microvascular Dermal EC none | 0.0 | 0.0 |
| Primary Tr1 act | 0.0 | 0.0 | Microsvasular Dermal EC TNFalpha + | 0.0 | 0.0 |
| | | | +IL-1beta | | |
| Primary Th1 rest | 0.0 | 0.0 | Bronchial epithelium TNFalpha + | 0.0 | 0.0 |
| | | | IL1beta | | |
| Primary Th2 rest | 0.0 | 0.0 | Small airway epithelium none | 0.0 | 0.0 |
| Primary Tr1 rest | 0.0 | 0.0 | Small airway epithelium TNFalpha + | 0.0 | 0.0 |
| | | | IL-1beta | | |
| CD45RA CD4 lymphocyte | 0.0 | 0.0 | Coronery artery SMC rest | 0.0 | 0.0 |
| act | | | | | |
| CD45RO CD4 lymphocyte | 0.0 | 0.0 | Coronery artery SMG TNFalpha + IL- | 0.0 | 0.0 |
| act | | | 1beta | | |
| CD8 lymphocyte act | 0.0 | 0.0 | Astrocytes rest | 8.3 | 0.0 |
| Secondary CD8 lymphocyte | 0.0 | 0.0 | Astrocytes TNFalpha + IL-1beta | 0.0 | 0.0 |
| rest | | | | | |
| Secondary CD8 lymphocyte | 0.0 | 0.0 | KU-812 (Basophil) rest | 0.0 | 0.0 |
| act | | | | | |
| CD4 lymphocyte none | 0.0 | 0.0 | KU-812 (Basophil) PMA/ionomycin | 0.0 | 0.0 |
| 2ry Th1/Th2/Tr1 anti-CD95 | 0.0 | 0.0 | CCD1106 (Keratinocytes) none | 0.0 | 0.0 |
| CH11 | | | | | |
| LAK cells rest | 0.0 | 0.0 | CCD1106 (Keratinocytes) TNFalpha | 0.0 | 0.0 |
| | | | + IL-1beta | | |
| LAK cells IL-2 | 0.0 | 0.0 | Liver cirrhosis | 0.0 | 0.0 |
| LAK cells IL-2 + IL-12 | 0.0 | 0.0 | NCI-H292 none | 0.0 | 0.0 |
| LAK cells IL-2 + IFN gamma | 0.0 | 0.0 | NCI-H292 IL-4 | 0.0 | 0.0 |
| LAK cells IL-2 + IL-18 | 0.0 | 0.0 | NCI-H292 IL-9 | 0.0 | 0.0 |
| LAK cells PMA/ionomycin | 0.0 | 0.0 | NCI-H292 IL-13 | 0.0 | 0.0 |
| NK Cells IL-2 rest | 0.0 | 0.3 | NCI-H292 IFN gamma | 0.0 | 0.0 |
| Two Way MLR 3 day | 0.0 | 0.0 | HPAEC none | 0.0 | 0.0 |
| Two Way MLR 5 day | 0.0 | 0.0 | HPAEC TNF alpha + IL-1 beta | 0.0 | 0.0 |
| Two Way MLR 7 day | 0.0 | 0.0 | Lung fibroblast none | 0.0 | 0.0 |
| PBMC rest | 0.0 | 0.0 | Lung fibroblast TNF alpha + IL-1 beta | 0.0 | 0.0 |
| PBMC PWM | 0.0 | 0.0 | Lung fibroblast IL-4 | 0.0 | 0.0 |
| PBMC PHA-L | 0.0 | 0.0 | Lung fibroblast IL-9 | 0.0 | 0.0 |
| Ramos (B cell) none | 0.0 | 0.0 | Lung fibroblast IL-13 | 0.0 | 0.0 |
| Ramos (B cell) ionomycin | 0.0 | 0.0 | Lung fibroblast IFN gamma | 0.0 | 0.0 |
| B lymphocytes PWM | 0.0 | 0.0 | Dermal fibroblast CCD1070 rest | 0.0 | 0.0 |
| B lymphocytes CD40L and | 0.0 | 0.0 | Dermal fibroblast CCD1070 TNF | 0.0 | 0.0 |
| IL-4 | | | alpha | | |
| EOL-1 dbcAMP | 0.0 | 0.0 | Dermal fibroblast CCD1070 IL-1 beta | 0.0 | 0.0 |
| EOL-1 dbcAMP | 0.0 | 0.0 | Dermal fibroblast IFN gamma | 0.0 | 0.0 |
| PMA/ionomycin | | | | | |
| Dendritic cells none | 0.0 | 0.0 | Dermal fibroblast IL-4 | 3.9 | 0.0 |
| Dendritic cells LPS | 0.0 | 0.0 | Dermal Fibroblasts rest | 0.0 | 0.0 |
| Dendritic cells anti-CD40 | 0.0 | 0.0 | Neutrophils TNFa + LPS | 17.9 | 0.0 |
| Monocytes rest | 0.0 | 0.0 | Neutrophils rest | 100.0 | 100.0 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 0.0 | Thymus | 0.0 | 0.0 |
| HUVEC none | 0.0 | 0.0 | Kidney | 0.0 | 0.0 |
| HUVEC starved | 0.0 | 0.0 | | | |

TABLE ATH
general oncology screening panel_v_2.4
Column A - Rel. Exp.(%) Ag4095, Run 268389981

| Tissue Name | A | Tissue Name | A |
|---|---|---|---|
| Colon cancer 1 | 50.7 | Bladder NAT 2 | 0.0 |
| CC Margin (ODO3921) | 0.0 | Bladder NAT 3 | 2.1 |
| Colon cancer 2 | 0.0 | Bladder NAT 4 | 0.0 |
| Colon NAT 2 | 0.0 | Prostate adenocarcinoma 1 | 0.0 |
| Colon cancer 3 | 21.9 | Prostate adenocarcinoma 2 | 0.0 |
| Colon NAT 3 | 0.0 | Prostate adenocarcinoma 3 | 0.0 |
| Colon malignant cancer 4 | 5.1 | Prostate adenocarcinoma 4 | 100.0 |
| Colon NAT 4 | 4.3 | Prostate NAT 5 | 0.0 |
| Lung cancer 1 | 0.0 | Prostate adenocarcinoma 6 | 0.0 |
| Lung NAT 1 | 0.0 | Prostate adenocarcinoma 7 | 0.0 |
| Lung cancer 2 | 0.0 | Prostate adenocarcinoma 8 | 0.0 |
| Lung NAT 2 | 0.0 | Prostate adenocarcinoma 9 | 0.0 |
| Squamous cell carcinoma 3 | 0.0 | Prostate NAT 10 | 0.0 |
| Lung NAT 3 | 0.0 | Kidney cancer 1 | 3.2 |
| Metastatic melanoma 1 | 11.0 | Kidney NAT 1 | 0.0 |
| Melanoma 2 | 0.0 | Kidney cancer 2 | 0.0 |
| Melanoma 3 | 0.0 | Kidney NAT 2 | 0.0 |
| Metastatic melanoma 4 | 0.0 | Kidney cancer 3 | 0.0 |
| Metastatic melanoma 5 | 7.0 | Kidney NAT 3 | 0.0 |
| Bladder cancer 1 | 0.0 | Kidney cancer 4 | 0.0 |
| Bladder NAT 1 | 0.0 | Kidney NAT 4 | 0.0 |
| Bladder cancer 2 | 0.0 | | |

General_screening_panel_v1.4 Summary: Ag4095 The expression of this gene was highest and almost exclusive to a sample derived from a colon cancer tissue (CT=27). Low to moderate levels of expression of this gene was also detected in two ovarian, two colon and a brain cancer cell lines. Expression levels of this gene is useful as marker to detect colon, ovarian, and brain cancers. Modulation of this gene, encoded protein and/or use of antibodies or small molecule drugs targeting this gene or gene product is useful in the treatment of colon ovarian, and brain cancers.

General_screening_panel_v1.5 Summary: Ag2888 Significant expression of the CG56142-01 gene was limited to cancer cell lines, with highest expression in a colon cancer cell line (CT=27.9). This gene encodes a putative prostasin, which has been identified as a potential marker of epithelial ovarian cancer. Based on the expression in these panels, expression of this gene will be used as a marker for colon cancer. Therapeutic modulation this gene, encoded protein and/or use of antibodies or small molecule targeting this gene or gene product is useful in the treatment of colon cancer.

Panel 2D Summary: Ag2888 The expression of the CG56142-01 gene appears to be highest and almost exclusive to a sample derived from a colon cancer (CT=30). Therapeutic modulation this gene, encoded protein and/or use of antibodies or small molecule targeting this gene or gene product is useful in the treatment of colon cancer.

Panel 3D Summary: Ag2888 The expression of the CG56142-01 gene was highest and almost exclusive to a sample derived from a gastric cancer cell line (CT=34.1). Thus, the expression of this gene is useful as marker for gastric cancer. Moreover, therapeutic modulation of this gene, throguh the use of small molecule drugs, antibodies or protein therapeutics might be beneficial in the treatment of gastric cancer.

Panel 4.1D Summary: Ag4095 This gene, which encodes a prostasin homolog, was expressed almost exclusively in resting neutrophils (CTs=31). This expression was down regulated in neutrophils activated by TNF-alpha and LPS. Modulation of this gene, encoded protein and/or use of agonist to activated this gene or gene product is useful to reduce activation of these inflammatory cells and eliminate the symptoms in patients with Crohn's disease, ulcerative colitis, multiple sclerosis, chronic obstructive pulmonary disease, asthma, emphysema, rheumatoid arthritis, lupus erythematosus, or psoriasis, AIDS or other immunodeficiencies.

general oncology screening panel_v_{—}2.4 Summary: Ag4095 Expression of this gene was highest in a prostate adenocarcinoma sample (CT=33). Expression of this gene was upregulated in 2/4 colon cancer samples compared to normal adjacent tissue. Therefore, expression of this gene is useful as a marker for colon cancer. Modulation of this gene, encoded protein and/or use of antibodies or small molecule drugs targeting this gene or gene product are beneficial in the treatment of colon cancer.

AV. CG56144-01: 7 Transmembrane Receptor.

Expression of gene CG56144-01 was assessed using the primer-probe sets Ag1221, Ag1221b and Ag1608, described in Tables AVA, AVB and AVC. Results of the RTQ-PCR runs are shown in Tables AVD, AVE, AVF and AVG.

TABLE AVA
Probe Name Ag1221

| | | | Start | SEQ ID |
|---|---|---|---|---|
| Primers | Sequences | Length | Position | No |
| Forward | 5′-ctacaaagcatttgggacatgt-3′ | 22 | 821 | 1374 |
| Probe | TET-5′-cataggtgccatcctgtccacctaca-3′- | 26 | 851 | 1375 |
| | TAMRA | | | |
| Reverse | 5′-ggtgcatgactgaagagatgac-3′ | 22 | 885 | 1376 |

TABLE AVB
Probe Name Ag1221b

| | | | Start | SEQ ID |
|---|---|---|---|---|
| Primers | Sequences | Length | Position | No |
| Forward | 5′-ctacacgacggtcctgactgggt-3′ | 23 | 512 | 1377 |
| Probe | TET-5′-catcaccaagattggcatggctgctgtg | 30 | 539 | 1378 |
| | gc-3′-TAMRA | | | |
| Reverse | 5′-aaggggagtggagtcattagtg-3′ | 22 | 580 | 1379 |

TABLE AVC
Probe Name Ag1608

| | | | Start | SEQ ID |
|---|---|---|---|---|
| Primers | Sequences | Length | Position | No |
| Forward | 5′-catattctggttcagggatcag-3′ | 22 | 368 | 1380 |
| Probe | TET-5′-caacttctttgcctgtctggtccaga-3′- | 26 | 395 | 1381 |
| | TAMRA | | | |
| Reverse | 5-′atggagaaggagtgaaggaaga-3′ | 22 | 424 | 1382 |

TABLE AVD
AI_comprehensive panel_v1.0
Column A - Rel. Exp.(%) Ag1221b, Run 228397224

| Tissue Name | A | Tissue Name | A |
|---|---|---|---|
| 110967 COPD-F | 10.8 | 112427 Match Control Psoriasis-F | 21.8 |
| 110980 COPD-F | 4.2 | 112418 Psoriasis-M | 43.8 |
| 110968 COPD-M | 10.0 | 112723 Match Control Psoriasis-M | 50.0 |
| 110977 COPD-M | 19.8 | 112419 Psoriasis-M | 18.3 |
| 110989 Emphysema-F | 22.8 | 112424 Match Control Psoriasis-M | 0.0 |
| 110992 Emphysema-F | 16.6 | 112420 Psoriasis-M | 34.2 |
| 110993 Emphysema-F | 42.3 | 112425 Match Control Psoriasis-M | 3.5 |
| 110994 Emphysema-F | 11.0 | 104689 (MF) OA Bone-Backus | 12.4 |
| 110995 Emphysema-F | 23.7 | 104690 (MF) Adj “Normal” Bone-Backus | 5.4 |
| 110996 Emphysema-F | 20.2 | 104691 (MF) OA Synovium-Backus | 42.0 |
| 110997 Asthma-M | 0.0 | 104692 (BA) OA Cartilage-Backus | 0.0 |
| 111001 Asthma-F | 9.9 | 104694 (BA) OA Bone-Backus | 11.5 |
| 111002 Asthma-F | 8.4 | 104695 (BA) Adj “Normal” Bone-Backus | 4.0 |
| 111003 Atopic Asthma-F | 6.0 | 104696 (BA) OA Synovium-Backus | 22.7 |
| 111004 Atopic Asthma-F | 0.0 | 104700 (SS) OA Bone-Backus | 85.9 |
| 111005 Atopic Asthma-F | 6.7 | 104701 (SS) Adj “Normal” Bone-Backus | 2.6 |
| 111006 Atopic Asthma-F | 0.0 | 104702 (SS) OA Synovium-Backus | 0.0 |
| 111417 Allergy-M | 1.8 | 117093 OA Cartilage Rep7 | 33.7 |
| 112347 Allergy-M | 1.8 | 112672 OA Bone5 | 16.3 |
| 112349 Normal Lung-F | 1.4 | 112673 OA Synovium5 | 13.9 |
| 112357 Normal Lung-F | 29.9 | 112674 OA Synovial Fluid cells5 | 12.1 |
| 112354 Normal Lung-M | 69.7 | 117100 OA Cartilage Rep14 | 0.0 |
| 112374 Crohns-F | 9.7 | 112756 OA Bone9 | 7.3 |
| 112389 Match Control Crohns-F | 9.0 | 112757 OA Synovium9 | 0.0 |
| 112375 Crohns-F | 0.0 | 112758 OA Synovial Fluid Cells9 | 0.0 |
| 112732 Match Control Crohns-F | 0.0 | 117125 RA Cartilage Rep2 | 19.3 |
| 112725 Crohns-M | 6.1 | 113492 Bone2 RA | 62.4 |
| 112387 Match Control Crohns-M | 9.2 | 113493 Synovium2 RA | 37.6 |
| 112378 Crohns-M | 13.4 | 113494 Syn Fluid Cells RA | 87.1 |
| 112390 Match Control Crohns-M | 11.0 | 113499 Cartilage4 RA | 89.5 |
| 112726 Crohns-M | 6.9 | 113500 Bone4 RA | 92.0 |
| 112731 Match Control Crohns-M | 22.7 | 113501 Synovium4 RA | 37.1 |
| 112380 Ulcer Col-F | 27.2 | 113502 Syn Fluid Cells4 RA | 24.8 |
| 112734 Match Control Ulcer Col-F | 25.2 | 113495 Cartilage3 RA | 53.2 |
| 112384 Ulcer Col-F | 45.1 | 113496 Bone3 RA | 59.5 |
| 112737 Match Control Ulcer Col-F | 10.0 | 113497 Synovium3 RA | 35.6 |
| 112386 Ulcer Col-F | 21.0 | 113498 Syn Fluid Cells3 RA | 100.0 |
| 112738 Match Control Ulcer Col-F | 15.4 | 117106 Normal Cartilage Rep20 | 0.0 |
| 112381 Ulcer Col-M | 2.6 | 113663 Bone3 Normal | 2.1 |
| 112735 Match Control Ulcer Col-M | 8.8 | 113664 Synovium3 Normal | 2.1 |
| 112382 Ulcer Col-M | 16.6 | 113665 Syn Fluid Cells3 Normal | 1.2 |
| 112394 Match Control Ulcer Col-M | 7.1 | 117107 Normal Cartilage Rep22 | 9.9 |
| 112383 Ulcer Col-M | 30.4 | 113667 Bone4 Normal | 3.5 |
| 112736 Match Control Ulcer Col-M | 4.5 | 113668 Synovium4 Normal | 11.8 |
| 112423 Psoriasis-F | 26.8 | 113669 Syn Fluid Cells4 Normal | 3.1 |

TABLE AVE
Panel 1.3D
Column A - Rel. Exp.(%) Ag1221b, Run 147341937
Column B - Rel. Exp.(%) Ag1221b, Run 152570853
Column C - Rel. Exp.(%) Ag1608, Run 147327901

| Tissue Name | A | B | C | Tissue Name | A | B | C |
|---|---|---|---|---|---|---|---|
| Liver adenocarcinoma | 0.0 | 0.0 | 0.0 | Kidney (fetal) | 1.6 | 0.0 | 0.0 |
| Pancreas | 0.0 | 0.0 | 0.0 | Renal ca. 786-0 | 0.0 | 0.0 | 0.0 |
| Pancreatic ca. CAPAN 2 | 0.0 | 0.0 | 0.0 | Renal ca. A498 | 1.6 | 2.5 | 0.9 |
| Adrenal gland | 1.2 | 4.2 | 0.0 | Renal ca. RXF 393 | 0.0 | 0.0 | 0.0 |
| Thyroid | 1.2 | 0.0 | 0.4 | Renal ca. ACHN | 0.0 | 0.0 | 0.0 |
| Salivary gland | 0.7 | 0.0 | 0.0 | Renal ca. UO-31 | 0.8 | 1.5 | 1.1 |
| Pituitaiy gland | 0.0 | 2.4 | 0.3 | Renal ca. TK-10 | 0.0 | 0.0 | 1.3 |
| Brain (fetal) | 0.0 | 0.0 | 0.0 | Liver | 2.3 | 5.2 | 1.2 |
| Brain (whole) | 1.6 | 3.6 | 0.0 | Liver (fetal) | 1.7 | 4.0 | 0.0 |
| Brain (amygdala) | 1.3 | 3.0 | 1.9 | Liver ca. (hepatoblast) | 0.0 | 0.0 | 0.0 |
| | | | | HepG2 | | | |
| Brain (cerebellum) | 0.0 | 1.3 | 0.0 | Lung | 6.3 | 16.7 | 5.8 |
| Brain (hippocampus) | 2.3 | 6.5 | 0.9 | Lung (fetal) | 2.0 | 8.1 | 3.0 |
| Brain (substantia nigra) | 2.4 | 3.9 | 2.6 | Lung ca. (small cell) LX-1 | 0.0 | 0.0 | 0.0 |
| Brain (thalamus) | 2.2 | 2.6 | 0.7 | Lung ca. (small cell) NCI- | 0.3 | 1.9 | 0.0 |
| | | | | H69 | | | |
| Cerebral Cortex | 1.6 | 5.5 | 0.7 | Lung ca. (s.cell var.) SHP- | 0.0 | 0.0 | 0.0 |
| | | | | 77 | | | |
| Spinal cord | 4.1 | 3.4 | 2.1 | Lung ca. (large cell) NCI- | 0.0 | 0.9 | 0.0 |
| | | | | H460 | | | |
| glio/astro U87-MG | 0.0 | 0.0 | 0.0 | Lung ca. (non-sm. cell) | 0.0 | 0.0 | 1.7 |
| | | | | A549 | | | |
| glio/astro U-118-MG | 0.5 | 0.0 | 2.7 | Lung ca. (non-s.cell) NCI- | 0.0 | 1.2 | 0.7 |
| | | | | H23 | | | |
| astrocytoma SW1783 | 0.0 | 0.0 | 0.0 | Lung ca. (non-s.cell) HOP- | 0.0 | 0.0 | 0.9 |
| | | | | 62 | | | |
| neuro*; met SK-N-AS | 0.3 | 0.0 | 0.0 | Lung ca. (non-s.cl) NCI- | 0.0 | 0.0 | 0.0 |
| | | | | H522 | | | |
| astrocytoma SF-539 | 0.0 | 2.4 | 0.0 | Lung ca. (squam.) SW 900 | 0.0 | 0.0 | 1.4 |
| astrocytoma SNB-75 | 0.6 | 0.0 | 0.0 | Lung ca. (squam.) NCI- | 1.1 | 0.0 | 0.0 |
| | | | | H596 | | | |
| glioma SNB-19 | 0.0 | 1.3 | 0.0 | Mammary gland | 0.6 | 0.0 | 1.1 |
| glioma U251 | 0.0 | 0.5 | 0.0 | Breast ca.* (pl.ef) MCF-7 | 0.0 | 0.0 | 0.0 |
| glioma SF-295 | 0.6 | 0.0 | 0.0 | Breast ca.* (pl.ef) MDA- | 0.0 | 0.0 | 0.0 |
| | | | | MB-231 | | | |
| Heart (Fetal) | 0.0 | 0.0 | 0.0 | Breast ca.* (pl.ef) T47D | 0.0 | 0.0 | 0.0 |
| Heart | 0.0 | 1.3 | 0.0 | Breast ca. BT-549 | 1.6 | 0.0 | 0.9 |
| Skeletal muscle (Fetal) | 2.9 | 7.7 | 2.0 | Breast ca. MDA-N | 0.0 | 0.0 | 0.4 |
| Skeletal muscle | 0.0 | 0.0 | 0.0 | Ovary | 4.4 | 6.3 | 3.4 |
| Bone marrow | 19.1 | 56.6 | 32.5 | Ovarian ca. OVCAR-3 | 0.0 | 0.0 | 0.0 |
| Thymus | 0.9 | 6.3 | 0.4 | Ovarian ca. OVCAR-4 | 0.0 | 0.0 | 0.0 |
| Spleen | 7.7 | 36.6 | 8.4 | Ovarian ca. OVCAR-5 | 0.4 | 0.0 | 2.2 |
| Lymph node | 0.7 | 1.5 | 0.6 | Ovarian ca. OVCAR-8 | 0.6 | 0.0 | 0.0 |
| Colorectal | 1.8 | 6.6 | 1.3 | Ovarian ca. IGROV-1 | 0.0 | 2.1 | 0.4 |
| Stomach | 0.3 | 0.8 | 0.5 | Ovarian ca. (ascites) SK- | 0.0 | 0.0 | 0.0 |
| | | | | OV-3 | | | |
| Small intestine | 1.0 | 1.2 | 0.7 | Uterus | 0.0 | 0.0 | 2.6 |
| Colon ca. SW480 | 0.0 | 0.0 | 0.0 | Placenta | 3.1 | 7.1 | 1.5 |
| Colon ca.* SW620 (SW480 | 0.0 | 0.0 | 0.0 | Prostate | 0.0 | 1.4 | 1.6 |
| met) | | | | | | | |
| Colon ca. HT29 | 0.5 | 1.2 | 0.0 | Prostate ca.* (bone met) | 0.0 | 0.0 | 0.0 |
| | | | | PC-3 | | | |
| Colon ca. HGT-116 | 0.0 | 1.1 | 0.0 | Testis | 1.2 | 2.4 | 1.0 |
| Colon ca. CaCo-2 | 0.0 | 3.4 | 0.0 | Melanoma Hs688(A).T | 0.0 | 0.0 | 0.0 |
| CC Well to Mod Duff | 3.1 | 3.8 | 3.9 | Melanoma* (met) | 0.6 | 0.0 | 0.0 |
| (ODO3866) | | | | Hs688(B).T | | | |
| Colon ca. HCC-2998 | 0.5 | 3.6 | 1.7 | Melanoma UACC-62 | 0.0 | 0.0 | 0.0 |
| Gastric ca. (liver met) NCI- | 100.0 | 100.0 | 100.0 | Melanoma M14 | 0.0 | 0.0 | 0.0 |
| N87 | | | | | | | |
| Bladder | 1.4 | 3.8 | 4.1 | Melanoma LOX IMVI | 1.1 | 0.0 | 0.0 |
| Trachea | 1.9 | 3.8 | 1.9 | Melanoma* (met) SK- | 0.0 | 0.0 | 0.0 |
| | | | | MEL-5 | | | |
| Kidney | 1.2 | 0.0 | 0.0 | Adipose | 0.9 | 14.1 | 8.4 |

TABLE AVF
Panel 2D
Column A - Rel. Exp.(%) Ag1221b, Run 150783273
Column B - Rel. Exp.(%) Ag1221b, Run 152570968

| Tissue Name | A | B | Tissue Name | A | B |
|---|---|---|---|---|---|
| Normal Colon | 27.4 | 12.9 | Kidney Margin 8120608 | 6.2 | 0.5 |
| CC Well to Mod Diff (OD03866) | 11.8 | 17.0 | Kidney Cancer 8120613 | 0.3 | 0.9 |
| CC Margin (ODO3866) | 20.2 | 7.0 | Kidney Margin 8120614 | 2.7 | 1.0 |
| CC Gr.2 rectosigmoid (ODO3868) | 0.0 | 2.0 | Kidney Cancer 9010320 | 30.8 | 20.6 |
| CC Margin (ODO3868) | 2.4 | 2.9 | Kidney Margin 9010321 | 5.3 | 2.8 |
| CC Mod Diff (ODO3920) | 12.9 | 2.5 | Normal Uterus | 0.0 | 0.0 |
| CC Margin (ODO3920) | 5.4 | 3.8 | Uterine Cancer 064011 | 11.7 | 5.3 |
| CC Gr.2 ascend colon (ODO3921) | 10.4 | 3.4 | Normal Thyroid | 2.7 | 0.0 |
| CC Margin (ODO3921) | 2.9 | 6.2 | Thyroid Cancer | 2.6 | 3.5 |
| CC from Partial Hepatectomy | 16.7 | 6.9 | Thyroid Cancer A302152 | 4.0 | 4.5 |
| (ODO4309) Mets | | | | | |
| Liver Margin (ODO4309) | 14.8 | 6.7 | Thyroid Margin A302153 | 3.7 | 5.2 |
| Colon mets to lung (OD04451-01) | 6.3 | 7.6 | Normal Breast | 5.4 | 6.8 |
| Lung Margin (OD04451-02) | 15.7 | 6.3 | Breast Cancer | 33.2 | 18.4 |
| Normal Prostate 6546-1 | 0.0 | 3.7 | Breast Cancer (OD04590-01) | 22.4 | 27.0 |
| Prostate Cancer (OD04410) | 7.8 | 10.4 | Breast Cancer Mets | 24.0 | 27.9 |
| | | | (OD04590-03) | | |
| Prostate Margin (OD04410) | 23.2 | 26.6 | Breast Cancer Metastasis | 5.1 | 6.1 |
| Prostate Cancer (OD04720-01) | 13.9 | 7.9 | Breast Cancer | 12.9 | 15.0 |
| Prostate Margin (OD04720-02) | 27.9 | 12.5 | Breast Cancer | 1.9 | 2.2 |
| Normal Lung | 100.0 | 100.0 | Breast Cancer 9100266 | 2.9 | 2.1 |
| Lung Met to Muscle (OD04286) | 15.9 | 7.1 | Breast Margin 9100265 | 2.4 | 1.2 |
| Muscle Margin (OD04286) | 13.9 | 13.9 | Breast Cancer A209073 | 13.7 | 5.8 |
| Lung Malignant Cancer | 29.3 | 24.0 | Breast Margin A209073 | 1.0 | 3.8 |
| (OD03126) | | | | | |
| Lung Margin (OD03126) | 61.6 | 53.6 | Normal Liver | 19.6 | 9.6 |
| Lung Cancer (OD04404) | 20.7 | 12.0 | Liver Cancer | 10.2 | 5.6 |
| Lung Margin (OD04404) | 28.7 | 25.7 | Liver Cancer 1025 | 2.9 | 5.0 |
| Lung Cancer (OD04565) | 1.3 | 2.8 | Liver Cancer 1026 | 13.7 | 14.7 |
| Lung Margin (OD04565) | 27.4 | 28.3 | Liver Cancer 6004-T | 3.2 | 11.3 |
| Lung Cancer (OD04237-01) | 2.5 | 8.5 | Liver Tissue 6004-N | 13.9 | 18.8 |
| Lung Margin (OD04237-02) | 39.0 | 36.9 | Liver Cancer 6005-T | 9.7 | 24.8 |
| Ocular Mel Met to Liver | 0.0 | 0.0 | Liver Tissue 6005-N | 2.1 | 3.4 |
| (ODO4310) | | | | | |
| Liver Margin (ODO4310) | 0.0 | 2.6 | Normal Bladder | 22.5 | 19.3 |
| Melanoma Metastasis | 0.0 | 1.1 | Bladder Cancer | 6.6 | 7.3 |
| Lung Margin (OD04321) | 33.4 | 31.2 | Bladder Cancer | 17.1 | 15.0 |
| Normal Kidney | 5.5 | 3.8 | Bladder Cancer (OD04718- | 39.0 | 29.5 |
| | | | 01) | | |
| Kidney Ca, Nuclear grade 2 | 45.7 | 22.7 | Bladder Normal Adjacent | 12.5 | 12.9 |
| (OD04338) | | | (OD04718-03) | | |
| Kidney Margin (OD04338) | 12.7 | 7.6 | Normal Ovary | 3.7 | 5.5 |
| Kidney Ca Nuclear grade 1/2 | 5.6 | 4.9 | Ovarian Cancer | 12.0 | 14.4 |
| (OD04339) | | | | | |
| Kidney Margin (OD04339) | 0.7 | 0.9 | Ovarian Cancer (OD04768- | 14.4 | 10.7 |
| | | | 07) | | |
| Kidney Ca, Clear cell type | 22.5 | 24.8 | Ovary Margin (OD04768-08) | 4.1 | 1.0 |
| (OD04340) | | | | | |
| Kidney Margin (OD04340) | 6.4 | 5.6 | Normal Stomach | 6.3 | 1.9 |
| Kidney Ca, Nuclear grade 3 | 17.6 | 21.2 | Gastric Cancer 9060358 | 1.4 | 3.4 |
| (OD04348) | | | | | |
| Kidney Margin (OD04348) | 26.6 | 24.8 | Stomach Margin 9060359 | 9.9 | 11.3 |
| Kidney Cancer (OD04622-01) | 53.6 | 59.0 | Gastric Cancer 9060395 | 12.7 | 7.7 |
| Kidney Margin (OD04622-03) | 2.2 | 1.9 | Stomach Margin 9060394 | 13.0 | 7.0 |
| Kidney Cancer (OD04450-01) | 1.3 | 0.0 | Gastric Cancer 9060397 | 28.3 | 18.9 |
| Kidney Margin (OD04450-03) | 2.5 | 7.3 | Stomach Margin 9060396 | 2.2 | 1.9 |
| Kidney Cancer 8120607 | 0.0 | 2.0 | Gastric Cancer 064005 | 31.9 | 27.2 |

TABLE AVG
Panel 4D
Column A - Rel. Exp.(%) Ag1221, Run 140237638
Column B - Ret. Exp.(%) Ag1221, Run 142014678
Column C - Ret. Exp.(%) Ag1221b, Run 150811715
Column D - Ret. Exp.(%) Ag1221b, Run 152571072
Column E - Ret. Exp.(%) Ag1221b, Run 158141668
Column F - Rel. Exp.(%) Ag1608, Run 149925800

| Tissue Name | A | B | C | D | E | F |
|---|---|---|---|---|---|---|
| Secondary Th1 act | 0.0 | 4.7 | 0.0 | 0.2 | 0.0 | 0.0 |
| Secondary Th2 act | 0.0 | 0.0 | 2.2 | 1.9 | 0.4 | 2.2 |
| Secondary Tr1 act | 0.0 | 0.0 | 0.0 | 0.3 | 0.0 | 0.7 |
| Secondary Th1 rest | 0.6 | 0.0 | 0.2 | 0.0 | 0.0 | 0.0 |
| Secondary Th2 rest | 0.0 | 0.0 | 0.0 | 0.0 | 0.0 | 0.0 |
| Secondary Tr1 rest | 0.0 | 0.0 | 0.0 | 0.0 | 0.0 | 0.8 |
| Primary Th1 act | 0.0 | 0.0 | 0.0 | 0.0 | 0.3 | 0.0 |
| Primary Th2 act | 0.0 | 0.0 | 0.0 | 0.0 | 0.0 | 0.4 |
| Primary Tr1 act | 0.0 | 2.6 | 0.2 | 0.0 | 0.1 | 0.0 |
| Primary Th1 rest | 3.1 | 0.0 | 0.7 | 0.6 | 0.6 | 0.6 |
| Primary Th2 rest | 0.0 | 2.7 | 0.0 | 0.0 | 0.0 | 0.0 |
| Primary Tr1 rest | 0.0 | 0.0 | 0.1 | 0.0 | 0.0 | 0.0 |
| CD45RA CD4 lymphocyte act | 1.4 | 0.0 | 0.9 | 3.6 | 0.8 | 2.1 |
| CD45RO CD4 lymphocyte act | 0.0 | 1.9 | 3.2 | 2.0 | 1.4 | 1.1 |
| CD8 lymphocyte act | 0.0 | 0.0 | 0.5 | 0.3 | 0.5 | 0.5 |
| Secondary CD8 lymphocyte rest | 0.0 | 0.0 | 0.7 | 2.8 | 1.2 | 1.6 |
| Secondary CD8 lymphocyte act | 0.0 | 0.0 | 0.0 | 0.0 | 0.0 | 0.0 |
| CD4 lymphocyte none | 1.8 | 0.0 | 0.9 | 0.0 | 0.9 | 3.5 |
| 2ry Th1/Th2/Tr1 anti-CD95 CH11 | 0.0 | 0.0 | 0.2 | 0.0 | 0.3 | 0.0 |
| LAK cells rest | 4.7 | 10.9 | 4.5 | 3.7 | 7.6 | 3.3 |
| LAK cells IL-2 | 0.0 | 2.3 | 1.3 | 3.5 | 1.9 | 2.1 |
| LAK cells IL-2 + IL-12 | 8.9 | 12.4 | 2.3 | 5.5 | 1.3 | 3.6 |
| LAK cells IL-2 + IFN gamma | 10.7 | 0.0 | 6.5 | 8.8 | 9.0 | 11.4 |
| LAK cells IL-2 + IL-18 | 5.8 | 5.31 | 2.5 | 9.6 | 4.8 | 7.3 |
| LAK cells PMA/ionomycin | 5.6 | 6.8 | 9.7 | 13.5 | 17.6 | 12.2 |
| NK Cells IL-2 rest | 1.5 | 2.2 | 0.4 | 0.7 | 0.0 | 0.2 |
| Two Way MLR 3 day | 21.9 | 33.0 | 17.3 | 20.4 | 32.1 | 19.6 |
| Two Way MLR 5 day | 4.3 | 7.0 | 4.9 | 5.2 | 6.7 | 1.8 |
| Two Way MLR 7 day | 2.7 | 0.0 | 0.7 | 1.8 | 0.9 | 0.8 |
| PBMC rest | 5.3 | 8.5 | 6.6 | 7.3 | 11.9 | 6.0 |
| PBMC PWM | 13.0 | 19.5 | 6.5 | 6.7 | 4.5 | 7.7 |
| PBMC PHA-L | 1.3 | 0.0 | 1.4 | 0.4 | 1.3 | 0.0 |
| Ramos (B cell) none | 0.0 | 0.0 | 0.0 | 0.0 | 0.7 | 0.0 |
| Ramos (B cell) ionomycin | 0.0 | 0.0 | 0.0 | 0.0 | 0.0 | 0.0 |
| B lymphocytes PWM | 4.2 | 4.7 | 0.7 | 0.3 | 0.5 | 1.9 |
| B lymphocytes CD40L and IL-4 | 0.0 | 0.0 | 0.0 | 0.1 | 0.2 | 0.3 |
| EOL-1 dbcAMP | 20.6 | 24.8 | 10.9 | 10.6 | 8.8 | 9.0 |
| EOL-1 dbcAMP PMA/ionomycin | 1.2 | 12.2 | 0.9 | 2.1 | 2.5 | 1.6 |
| Dendntic cells none | 0.0 | 0.0 | 0.8 | 0.9 | 1.4 | 1.1 |
| Dendritic cells LPS | 5.4 | 3.4 | 6.0 | 7.2 | 3.9 | 3.6 |
| Dendritic cells anti-CD40 | 4.9 | 19.2 | 6.0 | 6.4 | 4.3 | 6.7 |
| Monocytes rest | 100.0 | 94.6 | 100.0 | 100.0 | 100.0 | 100.0 |
| Monocytes LPS | 25.3 | 45.7 | 9.1 | 9.2 | 6.6 | 8.8 |
| Macrophages rest | 0.0 | 5.0 | 2.3 | 4.9 | 0.8 | 1.8 |
| Macrophages LPS | 58.2 | 100.0 | 25.0 | 32.5 | 25.9 | 21.0 |
| HUVEC none | 0.0 | 0.0 | 0.0 | 0.0 | 0.8 | 0.0 |
| HUVEC starved | 0.0 | 0.0 | 0.0 | 0.0 | 0.0 | 0.0 |
| HUVEC IL-1beta | 0.0 | 0.0 | 0.0 | 0.0 | 0.0 | 0.0 |
| HUVEC IFN gamma | 0.0 | 0.0 | 0.5 | 1.1 | 0.7 | 0.4 |
| HUVEC TNF alpha + IFN gamma | 0.0 | 0.0 | 0.2 | 0.5 | 0.3 | 0.3 |
| HUVEC TNF alpha + IL4 | 0.0 | 0.0 | 0.0 | 0.3 | 0.0 | 0.0 |
| HUVEC IL-11 | 0.0 | 0.0 | 0.0 | 0.0 | 0.0 | 0.0 |
| Lung Microvascular EC none | 0.0 | 0.0 | 0.2 | 0.0 | 1.3 | 0.0 |
| Lung Microvascular EC TNFalpha + IL-1beta | 0.0 | 0.0 | 0.4 | 0.6 | 0.3 | 0.3 |
| Microvascular Dermal EC none | 0.0 | 0.0 | 0.0 | 0.3 | 0.0 | 0.2 |
| Microsvasular Dermal EC TNFalpha + IL-1beta | 0.0 | 0.0 | 0.0 | 0.0 | 0.0 | 0.0 |
| Bronchial epithelium TNFalpha + IL1beta | 0.0 | 0.0 | 0.0 | 0.0 | 0.0 | 0.6 |
| Small airway epithelium none | 0.0 | 0.0 | 0.0 | 0.3 | 1.0 | 0.3 |
| Small airway epithelium TNFalpha + IL-1beta | 1.3 | 0.0 | 0.4 | 0.8 | 0.3 | 0.5 |
| Coronery artery SMC rest | 0.0 | 0.0 | 0.0 | 0.6 | 1.7 | 0.4 |
| Coronery artery SMC TNFalpha + IL-1beta | 0.0 | 0.0 | 0.0 | 0.0 | 0.0 | 0.4 |
| Astrocytes rest | 0.0 | 0.0 | 0.2 | 1.0 | 0.0 | 0.0 |
| Astrocytes TNFalpha + IL-1beta | 0.0 | 0.0 | 0.4 | 0.6 | 0.1 | 0.2 |
| KU-812 (Basophil) rest | 2.7 | 0.0 | 0.2 | 0.0 | 0.3 | 0.0 |
| KU-812 (Basophil) PMA/ionomycin | 1.2 | 1.4 | 0.7 | 0.6 | 0.5 | 0.6 |
| CCD1106 (Keratinocytes) none | 0.0 | 2.1 | 0.0 | 0.0 | 0.0 | 0.0 |
| 93580 CCD1106 (Keratinocytes) TNFa and IFNg | 6.3 | 13.3 | 0.2 | 0.0 | 1.1 | 0.4 |
| Liver cirrhosis | 15.1 | 20.0 | 4.6 | 10.2 | 7.4 | 5.0 |
| Lupus kidney | 0.0 | 0.0 | 0.0 | 0.0 | 0.0 | 0.0 |
| NCI-H292 none | 0.0 | 0.0 | 0.0 | 0.4 | 0.9 | 0.0 |
| NCI-H292 IL-4 | 0.0 | 0.0 | 0.0 | 0.0 | 0.0 | 0.1 |
| NCI-H292 IL-9 | 0.0 | 0.0 | 0.0 | 0.0 | 0.0 | 0.0 |
| NCI-H292 IL-13 | 0.0 | 0.0 | 0.0 | 0.0 | 0.0 | 0.0 |
| NCI-H292 IFN gamma | 0.0 | 1.7 | 0.4 | 1.0 | 0.4 | 0.4 |
| HPAEC none | 0.0 | 0.0 | 0.0 | 0.0 | 0.0 | 0.0 |
| HPAEC TNF alpha + IL-1 beta | 0.0 | 0.0 | 0.0 | 0.0 | 0.0 | 0.0 |
| Lung fibroblast none | 0.0 | 0.0 | 0.0 | 0.0 | 0.0 | 0.0 |
| Lung fibroblast TNF alpha + IL-1 beta | 0.9 | 0.0 | 0.2 | 0.6 | 0.3 | 0.5 |
| Lung fibroblast IL-4 | 0.0 | 0.0 | 0.0 | 0.3 | 0.3 | 0.0 |
| Lung fibroblast IL-9 | 0.0 | 0.0 | 0.0 | 0.0 | 0.3 | 0.0 |
| Lung fibroblast IL-13 | 0.0 | 0.0 | 0.0 | 0.6 | 0.0 | 0.0 |
| Lung fibroblast IFN gamma | 0.0 | 2.6 | 1.3 | 1.2 | 1.4 | 1.1 |
| Dermal fibroblast CCD1070 rest | 0.0 | 0.0 | 0.0 | 0.0 | 0.0 | 0.0 |
| Dermal fibroblast CCD1070 TNF alpha | 0.0 | 0.0 | 0.0 | 0.0 | 0.0 | 0.0 |
| Dermal fibroblast CCD1070 IL-1 beta | 0.0 | 0.0 | 0.0 | 0.0 | 0.0 | 0.0 |
| Dermal fibroblast IFN gamma | 1.6 | 0.0 | 0.2 | 1.1 | 0.3 | 0.9 |
| Dermal fibroblast IL-4 | 0.0 | 0.0 | 0.2 | 0.0 | 0.0 | 0.2 |
| IBD Colitis 2 | 0.0 | 0.0 | 0.0 | 0.0 | 0.0 | 0.0 |
| IBD Crohn's | 0.0 | 0.0 | 0.0 | 0.0 | 0.0 | 0.0 |
| Colon | 0.0 | 0.0 | 0.7 | 0.3 | 0.6 | 0.3 |
| Lung | 0.0 | 0.0 | 2.4 | 3.1 | 2.6 | 2.0 |
| Thymus | 0.0 | 0.0 | 0.4 | 0.6 | 1.0 | 0.4 |
| Kidney | 0.0 | 2.7 | 0.4 | 0.6 | 0.3 | 0.3 |

AI_comprehensive panel_v1.0 Summary: Ag1221b Highest expression of this gene was detected in synovial fluid from a rheumatoid arthritis (RA) patient (CT=33). This gene showed preferential expression in rheumatoid arthritis bone, cartilage, synovium and synovial fluid samples. Modulation of this gene, encoded protein and/or use of antibodies or small molecule drug targeting this gene or gene product is useful in the treatment of rheumatoid arthritis.

Panel 1.3D Summary: Ag1221b/Ag1608 The expression of this gene was seen predominantly in one gastric cancer cell line derived from a metastasis (CTs=30-32). This expression profile indicates that this gene plays a role in liver cancer metastasis to gastric. Significant expression was also seen in bone marrow and spleen indicating that this gene is also important in the hematopoietic system. Modulation of this gene, encoded protein and/or use of antibodies or small molecule drug are useful in treatment of gastric cancer, and disorders related to hematopoietic system.

Panel 2D Summary: Ag1221b Highest expression of this gene was detected in normal lung (CTs=31). Expression of this gene was downregulated in the lung cancers. Upregulation of this gene and/or use of agonist is useful in the treatment of lung cancers.

Significant expression of this gene was also seen in breast cancer, thyroid cancer, gastric cancer, ovarian cancer and renal cell carcinoma. Modulation of this gene, encoded protein and/or use of antibodies or small molecule drug targeting this gene or gene product is useful in the treatment of breast cancer, thyroid cancer, gastric cancer, ovarian cancer and renal cell carcinoma.

Panel 4D Summary: Ag1221b/Ag1608 Highest expression of this gene was detected in resting monocytes and LPS activated macrophages (CTs=28-33). The expression of this gene in resting monocytes indicated that this gene encoded a differentation antigen. Signalling through this molecule will stimulate activation. This gene was down regulated during activation of monocytes, but upregulated in activated macrophages indicating a role in antigen presentation. Significant expression of this gene was also detected in resting and activated LAK cells, two way MLR, resting eosinophils, activated PBMC and liver cirrhosis samples. Modulation of this gene, encoded protein and/or use of antibodies or small molecule targeting this gene or gene product will help reduce or eliminate inflammatory and autoimmune diseases such as asthma/allergy, emphysema, psoriasis, arthritis, IBD Colitis, liver cirrhosis.

AW. CG56146-01, CG56146-02 and CG5614603: 7 Transmembrane Receptor.

Expression of gene CG56146-01, CG56146-02 and CG56146-03 were assessed using the primer-probe sets Ag1175 and Ag1201, described in Tables AWA and AWB. Results of the RTQ-PCR runs are shown in Tables AWC, AWD, AWE and AWF. CG56146-02 and CG56146-03 represent full-length physical clones.

TABLE AWA
Probe Name Ag1175

| | | | Start | SEQ ID |
|---|---|---|---|---|
| Primers | Sequences | Length | Position | No |
| Forward | 5′-agagacaatccaaagccttttc-3′ | 22 | 551 | 1383 |
| Probe | TET-5′-caactgtgtgcctcacctcattgttg-3′- | 26 | 573 | 1384 |
| | TAMRA | | | |
| Reverse | 5′-agaccctggctttaaataagca-3′ | 22 | 627 | 1385 |

TABLE AWB
Probe Name Ag1201

| | | | Start | SEQ ID |
|---|---|---|---|---|
| Primers | Sequences | Length | Position | No |
| Forward | 5′-agagacaatccaaagccttttc-3′ | 22 | 551 | 1386 |
| Probe | TET-5′-caactgtgtgcctcacctcattgttg-3′- | 26 | 573 | 1387 |
| | TAMRA | | | |
| Reverse | 5′-agaccctggctttaaataagca-3′ | 22 | 627 | 1388 |

TABLE AWC
AI_comprehensive panel_v1.0
Column A - Rel. Exp.(%) Ag1175, Run 248064523
Column B - Rel. Exp.(%) Ag1201, Run 211193563
Column C - Rel. Exp.(%) Ag1201, Run 212308358

| Tissue Name | A | B | C | Tissue Name | A | B | C |
|---|---|---|---|---|---|---|---|
| 110967 COPD-F | 5.6 | 1.2 | 2.5 | 112427 Match Control | 76.3 | 42.9 | 98.6 |
| | | | | Psoriasis-F | | | |
| 110980 COPD-F | 5.0 | 0.0 | 0.0 | 112418 Psoriasis-M | 12.3 | 1.4 | 10.2 |
| 110968 COPD-M | 0.0 | 6.1 | 0.0 | 112723 Match Control | 0.0 | 0.0 | 0.0 |
| | | | | Psoriasis-M | | | |
| 110977 COPD-M | 14.7 | 12.5 | 9.5 | 112419 Psoriasis-M | 19.2 | 6.5 | 19.1 |
| 110989 Emphysema-F | 21.6 | 5.5 | 6.7 | 112424 Match Control | 5.6 | 1.8 | 9.4 |
| | | | | Psoriasis-M | | | |
| 110992 Emphysema-F | 0.0 | 8.7 | 3.6 | 112420 Psoriasis-M | 33.4 | 24.5 | 16.6 |
| 110993 Emphysema-F | 20.4 | 3.0 | 5.2 | 112425 Match Control | 58.6 | 100.0 | 87.7 |
| | | | | Psoriasis-M | | | |
| 110994 Emphysema-F | 8.1 | 0.0 | 0.0 | 104689 (MF) OA Bone- | 9.2 | 5.0 | 4.8 |
| | | | | Backus | | | |
| 110995 Emphysema-F | 18.9 | 4.1 | 10.1 | 104690 (MF) Adj “Normal” | 18.2 | 4.0 | 5.8 |
| | | | | Bone-Backus | | | |
| 110996 Emphysema-F | 3.7 | 0.0 | 0.0 | 104691 (MF) OA | 0.0 | 8.4 | 0.0 |
| | | | | Synovium-Backus | | | |
| 110997 Asthma-M | 20.9 | 0.0 | 13.5 | 104692 (BA) OA Cartilage- | 0.0 | 0.0 | 0.0 |
| | | | | Backus | | | |
| 111001 Asthma-F | 34.2 | 8.4 | 17.0 | 104694 (BA) OA Bone- | 7.7 | 0.0 | 8.1 |
| | | | | Backus | | | |
| 111002 Asthma-F | 30.8 | 5.9 | 23.8 | 104695 (BA) Adj “Normal” | 14.5 | 5.7 | 6.2 |
| | | | | Bone-Backus | | | |
| 111003 Atopic Asthma-F | 14.8 | 11.3 | 8.7 | 104696 (BA) OA | 13.4 | 19.2 | 6.3 |
| | | | | Synovium-Backus | | | |
| 111004 Atopic Asthma-F | 21.6 | 10.2 | 11.6 | 104700 (SS) OA Bone | 17.4 | 4.1 | 13.4 |
| | | | | Backus | | | |
| 111005 Atopic Asthma-F | 22.1 | 0.0 | 13.0 | 104701 (SS) Adj “Normal” | 13.7 | 7.6 | 3.3 |
| | | | | Bone-Backus | | | |
| 111006 Atopic Asthma-F | 5.8 | 0.0 | 4.7 | 104702 (SS) OA Synovium- | 8.6 | 12.5 | 4.0 |
| | | | | Backus | | | |
| 111417 Allergy-M | 1.5 | 11.7 | 17.9 | 117093 OA Cartilage Rep7 | 17.2 | 22.2 | 14.8 |
| 112347 Allergy-M | 0.0 | 0.0 | 0.0 | 112672 OA Bone5 | 15.0 | 3.0 | 14.6 |
| 112349 Normal Lung-F | 0.0 | 0.0 | 0.0 | 112673 OA Synovium5 | 26.8 | 2.7 | 3.8 |
| 112357 Normal Lung-F | 12.8 | 1.6 | 8.7 | 1112674 OA Synovial Fluid | 6.2 | 9.3 | 6.4 |
| 112354 Normal Lung-M | 10.5 | 0.0 | 11.8 | 17100 OA Cartilage | 1.9 | 11.0 | 4.2 |
| | | | | Rep14 | | | |
| 112374 Crohns-F | 12.7 | 10.7 | 12.6 | 112756 OA Bone9 | 100.0 | 60.3 | 100.0 |
| 112389 Match Control | 0.0 | 3.5 | 0.0 | 112757 OA Synovium9 | 0.0 | 0.0 | 0.0 |
| Crohns-F | | | | | | | |
| 112375 Crohns-F | 27.9 | 8.4 | 19.9 | 112758 OA Synovial Fluid | 0.0 | 7.2 | 12.2 |
| | | | | Cells9 | | | |
| 112732 Match Control | 0.0 | 1.8 | 5.4 | 117125 RA Cartilage Rep2 | 0.0 | 3.4 | 0.0 |
| 112725 Crohns-M | 5.4 | 3.3 | 4.1 | 113492 Bone2 RA | 0.0 | 6.8 | 0.0 |
| 112387 Match Control | 6.9 | 0.0 | 4.9 | 113493 Synovium2 RA | 0.0 | 0.0 | 0.0 |
| 112378 Crohns-M | 0.0 | 0.0 | 0.0 | 113494 Syn Fluid Cells RA | 0.0 | 0.0 | 4.2 |
| 112390 Match Control | 37.1 | 22.2 | 6.9 | 113499 Cartilage4 RA | 6.0 | 0.0 | 0.0 |
| Crohns-M | | | | | | | |
| 112726 Crohns-M | 3.3 | 0.0 | 0.0 | 113500 Bone4RA | 0.0 | 2.6 | 0.0 |
| 112731 Match Control | 0.0 | 3.1 | 15.8 | 113501 Synovium4 RA | 0.0 | 0.0 | 0.0 |
| Crohns-M | | | | | | | |
| 112380 Ulcer Col-F | 17.1 | 9.8 | 22.4 | 113502 Syn Fluid Cells4 | 0.0 | 5.1 | 3.6 |
| | | | | RA | | | |
| 112734 Match Control | 5.8 | 1.2 | 0.0 | 113495 Cartilage3 RA | 0.0 | 1.2 | 3.6 |
| Ulcer Col-F | | | | | | | |
| 112384 Ulcer Col-F | 39.2 | 6.1 | 11.3 | 113496 Bone3 RA | 3.5 | 0.0 | 0.0 |
| 112737 Match Control | 4.1 | 0.0 | 12.7 | 113497 Synovium3 RA | 0.0 | 0.0 | 0.0 |
| Ulcer Col-F | | | | | | | |
| 112386 Ulcer Col-F | 0.0 | 5.9 | 0.0 | 113498 Syn Fluid Cells3 | 0.0 | 0.0 | 0.0 |
| | | | | RA | | | |
| 112738 Match Control | 0.0 | 0.0 | 6.3 | 117106 Normal Cartilage | 0.0 | 3.9 | 0.0 |
| Ulcer Col-F | | | | Rep20 | | | |
| 112381 Ulcer Col-M | 0.0 | 0.4 | 0.6 | 113663 Bone3 Normal | 0.0 | 0.0 | 0.0 |
| 112735 Match Control | 3.6 | 1.0 | 6.6 | 113664 Synovium3 Normal | 0.0 | 0.0 | 0.0 |
| Ulcer Col-M | | | | | | | |
| 112382 Ulcer Col-M | 5.1 | 4.7 | 3.9 | 113665 Syn Fluid Cells3 | 0.0 | 0.0 | 0.7 |
| | | | | Normal | | | |
| 112394 Match Control | 0.0 | 2.1 | 0.0 | 117107 Normal Cartilage | 8.7 | 9.7 | 11.9 |
| Ulcer Col-M | | | | Rep22 | | | |
| 112383 Ulcer Col-M | 2.5 | 0.0 | 0.0 | 113667 Bone4 Normal | 6.3 | 10.2 | 15.0 |
| 112736 Match Control | 0.0 | 0.0 | 0.0 | 113668 Synovium4 Normal | 0.0 | 0.0 | 10.8 |
| Ulcer Col-M | | | | | | | |
| 112423 Psoriasis-F | 17.9 | 24.8 | 44.1 | 113669 SynFluid Cells4 | 21.8 | 9.6 | 23.2 |
| | | | | Normal | | | |

TABLE AWD
Panel 1.3D
Column A - Rel. Exp.(%) Ag1201, Run 147490505
Column B - Rel. Exp.(%) Ag1201, Run 148438245
Column C - Rel. Exp.(%) Ag1201, Run 152628232

| Tissue Name | A | B | C | Tissue Name | A | B | C |
|---|---|---|---|---|---|---|---|
| Liver adenocarcinoma | 0.0 | 0.0 | 13.7 | Kidney (fetal) | 15.7 | 0.0 | 19.6 |
| Pancreas | 0.0 | 0.0 | 0.0 | Renal ca. 786-0 | 0.0 | 0.0 | 0.0 |
| Pancreatic ca. CAPAN 2 | 0.0 | 0.0 | 0.0 | Renal ca. A498 | 0.0 | 0.0 | 0.0 |
| Adrenal gland | 0.0 | 0.0 | 0.0 | Renal ca. RXF 393 | 0.0 | 0.0 | 0.0 |
| Thyroid | 0.0 | 0.0 | 26.2 | Renal ca. ACHN | 0.0 | 0.0 | 0.0 |
| Salivary gland | 0.0 | 0.0 | 14.7 | Renal ca. UO-31 | 0.0 | 0.0 | 0.0 |
| Pituitary gland | 7.5 | 25.3 | 0.0 | Renal ca. TK-10 | 0.0 | 0.0 | 0.0 |
| Brain (fetal) | 0.0 | 13.5 | 0.0 | Liver | 0.0 | 0.0 | 0.0 |
| Brain (whole) | 3.3 | 0.0 | 0.0 | Liver (fetal) | 0.0 | 0.0 | 0.0 |
| Brain (amygdala) | 0.0 | 0.0 | 0.0 | Liver ca. (hepatoblast) | 0.0 | 0.0 | 0.0 |
| | | | | HepG2 | | | |
| Brain (cerebellum) | 0.0 | 0.0 | 0.0 | Lung | 9.2 | 0.0 | 0.0 |
| Brain (hippocampus) | 0.0 | 10.6 | 0.0 | Lung (fetal) | 0.0 | 0.0 | 0.0 |
| Brain (substantia nigra) | 0.0 | 0.0 | 0.0 | Lung ca. (small cell) LX-1 | 0.0 | 0.0 | 0.0 |
| Brain (thalamus) | 0.0 | 0.0 | 0.0 | Lung ca. (small cell) NCI-H69 | 0.0 | 0.0 | 0.0 |
| Cerebral Cortex | 0.0 | 0.0 | 0.0 | Lung ca. (s.cell var.) SHP-77 | 0.0 | 0.0 | 0.0 |
| Spinal cord | 8.3 | 0.0 | 0.0 | Lung ca. (large cell) NCI-H460 | 0.0 | 0.0 | 0.0 |
| glio/astro U87-MG | 0.0 | 0.0 | 0.0 | Lung ca. (non-sm. cell) A549 | 1.5 | 0.0 | 0.0 |
| gilo/astro U-118-MG | 0.0 | 0.0 | 0.0 | Lung ca. (non-s.cell) NCI-H23 | 6.2 | 26.6 | 94.0 |
| astrocytoma SW1783 | 0.0 | 0.0 | 0.0 | Lung ca. (non-s.cell) HOP-62 | 20.9 | 23.8 | 27.0 |
| neuro*; met SK-N-AS | 0.0 | 0.0 | 0.0 | Lung ca. (non-s.cl) NCI-H522 | 0.0 | 0.0 | 0.0 |
| astrocytoma SF-539 | 0.0 | 0.0 | 15.6 | Lung ca. (squam.) SW 900 | 0.0 | 0.0 | 0.0 |
| astrocytoma SNB-75 | 0.0 | 0.0 | 0.0 | Lung ca. (squam.) NCI-H596 | 0.0 | 0.0 | 0.0 |
| glioma SNB-19 | 0.0 | 0.0 | 12.0 | Mammary gland | 15.6 | 0.0 | 12.9 |
| glioma U251 | 0.0 | 0.0 | 0.0 | Breast ca.* (pl.ef) MCF-7 | 0.0 | 0.0 | 0.0 |
| glioma SF-295 | 0.0 | 0.0 | 0.0 | Breast ca.* (pl.ef) MDA- | 0.0 | 0.0 | 0.0 |
| | | | | MB-231 | | | |
| Heart (Fetal) | 0.0 | 0.0 | 0.0 | Breast ca.* (pl. ef) T47D | 0.0 | 0.0 | 0.0 |
| Heart | 7.7 | 0.0 | 0.0 | Breast ca. BT-549 | 6.7 | 0.0 | 0.0 |
| Skeletal muscle (Fetal) | 11.2 | 28.1 | 10.7 | Breast ca. MDA-N | 0.0 | 0.0 | 0.0 |
| Skeletal muscle | 0.0 | 0.0 | 0.0 | Ovary | 0.0 | 0.0 | 0.0 |
| Bone marrow | 0.0 | 9.9 | 20.7 | Ovarian ca. OVCAR-3 | 6.2 | 0.0 | 0.0 |
| Thymus | 8.6 | 0.0 | 0.0 | Ovarian ca. OVCAR-4 | 0.0 | 0.0 | 0.0 |
| Spleen | 11.3 | 0.0 | 28.1 | Ovarian ca. OVCAR-5 | 0.0 | 0.0 | 0.0 |
| Lymph node | 7.4 | 0.0 | 0.0 | Ovarian ca. OVCAR-8 | 33.7 | 36.3 | 83.5 |
| Colorectal | 10.8 | 30.1 | 0.0 | Ovarian ca. IGROV-1 | 0.0 | 0.0 | 15.3 |
| Stomach | 5.9 | 0.0 | 12.4 | Ovarian ca. (ascites) SK- | 0.0 | 5.9 | 0.0 |
| | | | | OV-3 | | | |
| Small intestine | 0.0 | 9.8 | 11.7 | Uterus | 0.0 | 20.0 | 0.0 |
| Colon ca. SW480 | 0.0 | 0.0 | 0.0 | Placenta | 0.0 | 0.0 | 0.0 |
| Colon ca.* SW620 | 0.0 | 0.0 | 0.0 | Prostate | 0.0 | 12.6 | 22.2 |
| (SW480 met) | | | | | | | |
| Colon ca. HT29 | 0.0 | 0.0 | 0.0 | Prostate ca.* (bone met) | 14.4 | 50.0 | 19.5 |
| | | | | PC-3 | | | |
| Colon ca. HCT-116 | 0.0 | 0.0 | 0.0 | Testis | 100.0 | 100.0 | 100.0 |
| Colon ca. CaCo-2 | 0.0 | 0.0 | 0.0 | Melanoma Hs688(A).T | 0.0 | 0.0 | 0.0 |
| CC Well to Mod Diff | 0.0 | 13.6 | 0.0 | Melanoma* (met) | 6.9 | 0.0 | 0.0 |
| (ODO3866) | | | | Hs688(B).T | | | |
| Colon ca. HCC-2998 | 0.0 | 0.0 | 0.0 | Melanoma UACC-62 | 0.0 | 0.0 | 0.0 |
| Gastric ca. (liver met) | 0.0 | 0.0 | 0.0 | Melanoma M14 | 0.0 | 0.0 | 0.0 |
| NCI-N87 | | | | | | | |
| Bladder | 0.0 | 0.0 | 0.0 | Melanoma LOX IMVI | 0.0 | 8.0 | 0.0 |
| Trachea | 71.2 | 0.0 | 0.0 | Melanoma* (met) SK-MEL-5 | 0.0 | 0.0 | 0.0 |
| Kidney | 0.0 | 0.0 | 0.0 | Adipose | 0.0 | 15.0 | 12.0 |

TABLE AWE
Panel 2D
Column A - Rel. Exp.(%) Ag1201, Run 147490530
Column B - Rel. Exp.(%) Ag1201, Run 148032505

| Tissue Name | A | B | Tissue Name | A | B |
|---|---|---|---|---|---|
| Normal Colon | 14.2 | 12.9 | Kidney Margin 8120608 | 0.0 | 10.0 |
| CC Well to Mod Diff (ODO3866) | 10.1 | 14.6 | Kidney Cancer 8120613 | 0.0 | 4.0 |
| CC Margin (ODO3866) | 0.0 | 10.7 | Kidney Margin 8120614 | 0.0 | 0.0 |
| CC Gr.2 rectosigmoid (ODO3868) | 0.0 | 4.5 | Kidney Cancer 9010320 | 0.0 | 0.0 |
| CC Margin (ODO3868) | 0.0 | 4.9 | Kidney Margin 9010321 | 0.0 | 0.0 |
| CC Mod Diff (ODO3920) | 0.0 | 5.4 | Normal Uterus | 0.0 | 3.6 |
| CC Margin (ODO3920) | 13.4 | 5.6 | Uterine Cancer 064011 | 66.4 | 54.0 |
| CC Gr.2 ascend colon (ODO3921) | 4.0 | 3.3 | Normal Thyroid | 9.5 | 0.0 |
| CC Margin (ODO3921) | 5.1 | 18.7 | Thyroid Cancer | 0.0 | 3.4 |
| CC from Partial Hepatectomy | 0.0 | 0.0 | Thyroid Cancer A302152 | 11.9 | 33.0 |
| (ODO4309) Mets | | | | | |
| Liver Margin (ODO4309) | 0.0 | 0.0 | Thyroid Margin A302153 | 28.5 | 39.5 |
| Colon mets to lung (OD04451-01) | 4.3 | 0.0 | Normal Breast | 18.2 | 28.3 |
| Lung Margin (OD04451-02) | 5.0 | 0.0 | Breast Cancer | 42.3 | 65.5 |
| Normal Prostate 6546-1 | 0.0 | 12.8 | Breast Cancer (OD04590-01) | 0.0 | 0.0 |
| Prostate Cancer (OD04410) | 100.0 | 100.0 | Breast Cancer Mets | 0.0 | 0.0 |
| | | | (OD04590-03) | | |
| Prostate Margin (OD04410) | 8.0 | 9.6 | Breast Cancer Metastasis | 31.6 | 35.6 |
| Prostate Cancer (OD04720-01) | 50.0 | 64.2 | Breast Cancer | 18.4 | 28.7 |
| Prostate Margin (OD04720-02) | 47.0 | 29.7 | Breast Cancer | 37.6 | 35.8 |
| Normal Lung | 2.8 | 10.6 | Breast Cancer 9100266 | 31.4 | 36.9 |
| Lung Met to Muscle (OD04286) | 3.9 | 11.1 | Breast Margin 9100265 | 14.5 | 48.3 |
| Muscle Margin (OD04286) | 0.0 | 0.0 | Breast Cancer A209073 | 51.8 | 68.3 |
| Lung Malignant Cancer | 0.0 | 0.0 | Breast Margin A209073 | 41.5 | 22.7 |
| (OD03126) | | | | | |
| Lung Margin (OD03126) | 0.0 | 5.2 | Normal Liver | 113.5 | 0.0 |
| Lung Cancer (OD04404) | 28.5 | 6.0 | Liver Cancer | 0.0 | 0.0 |
| Lung Margin (OD04404) | 5.4 | 19.2 | Liver Cancer 1025 | 4.7 | 5.0 |
| Lung Cancer (OD04565) | 0.0 | 0.0 | Liver Cancer 1026 | 0.0 | 0.0 |
| Lung Margin (OD04565) | 1.8 | 0.0 | Liver Cancer 6004-T | 0.0 | 3.8 |
| Lung Cancer (OD04237-01) | 0.0 | 0.0 | Liver Tissue 6004-N | 3.6 | 0.0 |
| Lung Margin (OD04237-02) | 6.2 | 4.9 | Liver Cancer 6005-T | 0.0 | 0.0 |
| Ocular Mel Met to Liver | 0.0 | 10.4 | Liver Tissue 6005-N | 0.0 | 0.0 |
| (ODO4310) | | | | | |
| Liver Margin (ODO4310) | 2.5 | 3.5 | Normal Bladder | 9.8 | 11.2 |
| Melanoma Metastasis | 0.0 | 0.0 | Bladder Cancer | 0.0 | 0.0 |
| Lung Margin (OD04321) | 0.0 | 0.0 | Bladder Cancer | 5.5 | 24.1 |
| Normal Kidney | 6.3 | 11.7 | Bladder Cancer (OD04718- | 0.0 | 0.0 |
| | | | 01) | | |
| Kidney Ca, Nuclear grade 2 | 47 | 13.1 | Bladder Normal Adjacent | 50 | 13.6 |
| (OD04338) | | | (OD04718-03) | | |
| Kidney Margin (OD04338) | 0.0 | 0.0 | Normal Ovary | 0.0 | 0.0 |
| Kidney Ca Nuclear grade 1/2 | 29.5 | 94.6 | Ovarian Cancer | 20.6 | 0.0 |
| (OD04339) | | | | | |
| Kidney Margin (OD04339) | 0.0 | 4.9 | Ovarian Cancer (OD04768- | 92.7 | 99.3 |
| | | | 07) | | |
| Kidney Ca, Clear cell type | 0.0 | 17.1 | Ovary Margin (OD04768-08) | 0.0 | 9.5 |
| (OD04340) | | | | | |
| Kidney Margin (OD04340) | 3.6 | 0.0 | Normal Stomach | 0.0 | 5.3 |
| Kidney Ca, Nuclear grade 3 | 0.0 | 0.0 | Gastric Cancer 9060358 | 0.0 | 0.0 |
| (OD04348) | | | | | |
| Kidney Margin (OD04348) | 7.9 | 5.1 | Stomach Margin 9060359 | 0.0 | 0.0 |
| Kidney Cancer (OD04622-01) | 7.4 | 0.0 | Gastric Cancer 9060395 | 10.4 | 0.0 |
| Kidney Margin (OD04622-03) | 0.0 | 0.0 | Stomach Margin 9060394 | 2.6 | 0.0 |
| Kidney Cancer (OD04450-01) | 0.0 | 0.0 | Gastric Cancer 9060397 | 4.3 | 0.0 |
| Kidney Margin (OD04450-03) | 0.0 | 0.0 | Stomach Margin 9060396 | 0.0 | 0.0 |
| Kidney Cancer 8120607 | 0.0 | 0.0 | Gastric Cancer 064005 | 4.8 | 0.0 |

TABLE AWF
Panel 4D
Column A - Rel. Exp.(%) Ag1175, Run 139801091
Column B - Rel. Exp.(%) Ag1201, Run 140237534
Column C - Rel. Exp.(%) Ag1201, Run 144180636

| Tissue Name | A | B | C | Tissue Name | A | B | C |
|---|---|---|---|---|---|---|---|
| Secondary Th1 act | 0.0 | 0.0 | 0.0 | HUVEC IL-1beta | 0.0 | 0.0 | 0.0 |
| Secondary Th2 act | 0.0 | 0.0 | 0.0 | HUVEC IFN gamma | 0.0 | 0.0 | 0.0 |
| Secondary Tr1 act | 0.0 | 0.0 | 0.0 | HUVEC TNF alpha + IFN | 0.0 | 0.0 | 0.0 |
| | | | | gamma | | | |
| Secondary Th1 rest | 0.0 | 0.0 | 0.0 | HUVEC TNF alpha + IL4 0.0 | 0.0 | 0.0 | |
| Secondary Th2 rest | 0.0 | 0.0 | 0.0 | HUVEC IL-11 | 0.0 | 0.0 | 0.0 |
| Secondary Tr1 rest | 0.0 | 0.0 | 0.0 | Lung Microvascular EC none | 0.0 | 0.0 | 0.0 |
| Primary Th1 act | 0.0 | 0.0 | 0.0 | Lung Microvascular EC | 0.0 | 0.0 | 0.0 |
| | | | | TNFalpha + IL-1beta | | | |
| Primary Th2 act | 0.0 | 0.0 | 0.0 | Microvascular Dermal EC | 0.0 | 5.4 | 0.0 |
| Primary Tr1 act | 0.0 | 0.0 | 0.0 | Microsvasular Dermal EC | 0.0 | 0.0 | 0.0 |
| | | | | TNFalpha + IL-1beta | | | |
| Primary Th1 rest | 0.0 | 0.0 | 0.0 | Bronchial epithelium | 0.0 | 0.0 | 0.0 |
| | | | | TNFalpha + IL1beta | | | |
| Primary Th2 rest | 0.0 | 0.0 | 0.0 | Small airway epithelium none | 0.0 | 0.0 | 0.0 |
| Primary Tr1 rest | 0.0 | 0.0 | 0.0 | Small airway epithelium | 0.0 | 0.0 | 0.0 |
| | | | | TNFalpha + IL-1beta | | | |
| CD45RA CD4 | 0.0 | 0.0 | 0.0 | Coronery artery SMC rest 0.0 | 0.0 | 0.0 | |
| lymphocyte act | | | | | | | |
| CD45RO CD4 | 0.0 | 0.0 | 0.0 | Coronery artery SMC | 0.0 | 0.0 | 0.0 |
| lymphocyte act | | | | TNFalpha + IL-1beta | | | |
| CD8 lymphocyte act | 0.0 | 2.3 | 0.0 | Astrocytes rest | 0.0 | 0.0 | 0.0 |
| Secondary CD8 | 3.6 | 0.0 | 0.0 | Astrocytes TNFalpha + IL- | 0.0 | 4.3 | 0.0 |
| lymphocyte rest | | | | 1beta | | | |
| Secondary CD8 | 0.0 | 0.0 | 0.0 | KU-812 (Basophil) rest | 15.8 | 7.9 | 29.1 |
| lymphocyte act | | | | | | | |
| CD4 lymphocyte none | 0.0 | 0.0 | 0.0 | KU-812 (Basophil) | 100.0 | 100.0 | 100.0 |
| | | | | PMA/ionomycin | | | |
| 2ry Th1/Th2/Tr1 anti- | 0.0 | 0.0 | 0.0 | CCD1106 (Keratinocytes) | 0.0 | 0.0 | 0.0 |
| CD95 CH11 | | | | none | | | |
| LAK cells rest | 0.0 | 3.3 | 4.4 | 93580 CCD1106 | 0.0 | 0.0 | 0.0 |
| | | | | (Keratinocytes) TNFa and | | | |
| | | | | IFNg | | | |
| LAK cells IL-2 | 3.4 | 0.0 | 0.0 | Liver cirrhosis | 16.4 | 11.3 | 15.2 |
| LAK cells IL-2 + IL-12 | 0.0 | 0.0 | 0.0 | Lupus kidney | 0.0 | 0.0 | 0.0 |
| LAX cells IL-2 + IFN | 0.0 | 0.0 | 0.0 | NCI-H292 none | 0.0 | 0.0 | 0.0 |
| gamma | | | | | | | |
| LAK cells IL-2 + IL-18 | 0.0 | 0.0 | 0.0 | NCI-H292 IL-4 | 0.0 | 0.0 | 0.0 |
| LAK Cells | 3.5 | 0.0 | 0.0 | NCI-H292 IL-9 | 0.0 | 0.0 | 0.0 |
| PMA/ionomycin | | | | | | | |
| NK Cells IL-2 rest | 0.0 | 0.0 | 0.0 | NCI-H292 IL-13 | 0.0 | 0.0 | 0.0 |
| Two Way MLR 3 day | 0.0 | 3.4 | 0.0 | NCI-H292 IFN gamma | 0.0 | 0.0 | 0.0 |
| Two Way MLR 5 day | 0.0 | 8.3 | 0.0 | HPAEC none | 0.0 | 0.0 | 0.0 |
| Two Way MLR 7 day | 0.0 | 0.0 | 0.0 | HPAEC TNF alpha + IL-1 | 0.0 | 0.0 | 0.0 |
| | | | | beta | | | |
| PBMC rest | 0.0 | 0.0 | 0.0 | Lung fibroblast none | 0.0 | 0.0 | 0.0 |
| PBMC PWM | 0.0 | 0.0 | 0.0 | Lung fibroblast TNF alpha + | 0.0 | 0.0 | 0.0 |
| | | | | IL-1 beta | | | |
| PBMC PHA-L | 0.0 | 0.0 | 0.0 | Lung fibroblast IL-4 | 0.0 | 0.0 | 0.0 |
| Ramos (B cell) none | 0.0 | 0.0 | 0.0 | Lung fibroblast IL-9 | 0.0 | 0.0 | 0.0 |
| Ramos (B cell) | 0.0 | 0.0 | 0.0 | Lung fibroblast IL-13 | 0.0 | 0.0 | 0.0 |
| ionomycin | | | | | | | |
| B lymphocytes PWM | 0.0 | 0.0 | 0.0 | Lung fibroblast IFN gamma | 0.0 | 0.0 | 0.0 |
| B lymphocytes CD40L | 0.0 | 0.0 | 0.0 | Dermal fibroblast CCD1070 | 6.2 | 0.0 | 0.0 |
| and IL-4 | | | | rest | | | |
| EOL-1 dbcAMP | 0.0 | 0.0 | 0.0 | Dermal fibroblast CCD1070 | 0.0 | 0.0 | 0.0 |
| | | | | TNF alpha | | | |
| EOL-1 dbcAMP | 0.0 | 0.0 | 0.0 | Dermal fibroblast CCD1070 | 0.0 | 0.0 | 0.0 |
| PMA/ionomycin | | | | IL-1 beta | | | |
| Dendritic cells none | 0.0 | 0.0 | 4.5 | Dermal fibroblast IFN gamma | 0.0 | 0.0 | 0.0 |
| Dendritic cells LPS | 0.0 | 10.0 | 0.0 | Dermal fibroblast IL-4 | 0.0 | 0.0 | 0.0 |
| Dendritic cells anti | 0.0 | 7.9 | 4.7 | IBD Colitis 2 | 3.1 | 5.3 | 0.0 |
| CD40 | | | | | | | |
| Monocytes rest | 0.0 | 0.0 | 0.0 | IBD Crohn's | 0.0 | 0.0 | 0.0 |
| Monocytes LPS | 0.0 | 0.0 | 0.0 | Colon | 2.4 | 0.0 | 0.0 |
| Macrophages rest | 4.1 | 3.7 | 13.4 | Lung | 14.9 | 3.1 | 0.0 |
| Macrophages LPS | 10.3 | 0.0 | 4.2 | Thymus | 9.6 | 7.4 | 12.0 |
| HUVEC none | 0.0 | 0.0 | 0.0 | Kidney | 0.0 | 3.9 | 3.6 |
| HUVEC starved | 6.3 | 0.0 | 0.0 | | | | |

AI_comprehensive panel_v1.0 Summary: Ag1175/Ag1201 Highest expression of this gene was detected in orthoarthritis bone and matched control psoriasis sample (CTs=32-33.5). Significant expression of this gene was also seen in psoriasis and asthma samples. Modulation of this gene, encoded protein and/or use of antibodies or small molecule drug targeting this gene or gene product is useful in the treatment of orthoarthritis, asthma and psoriasis.

Panel 1.3D Summary: Ag1201 This gene showed significant expression mainly in testis (CTs=33-34). Modulation of this gene and encoded protein is useful in the treatment of testis related disorders such as fertility and hypogonadism.

Panel 2D Summary: Ag1201 Highest expression of this gene was detected in prostate cancer sample (CTs=32). This gene was over-expressed in tumors derived from tissues responsive to steroid hormones—ovarian, uterine and prostate cancers. Expression level of this gene is useful as a marker to detect tumor cells responsive to steroid hormones and to differentiate hormone-responsive and non-hormone responsive tumors that are known to lead to different clinical outcomes. Modulation of this gene, encoded protein and/or use of antibodies or small molecule drug targeting this gene or gene product is useful in the treatment of ovarian, uterine and prostate cancers.

Panel 4D Summary: Ag1201 This gene showed low expression in activated basophils (CTs=32-33). Basophils are one of the key cell mediators of inflammation during asthma and allergy (Oliver J M, Kepley C L, Ortega E, Wilson B S, 2000, Immunologically mediated signaling in basophils and mast cells: finding therapeutic targets for allergic diseases in the human Fcvar epsilonR1 signaling pathway. Immunopharmacology 48(3):269-81). This expression indicated that this gene has a potential role in inflammation and helps the basophils to extravasate into the site of inflammation and/or in the activation of these cells. Modulation of this gene, encoded protein and/or use of antibodies or small molecule drug targeting this gene or gene product is useful to inhibit nasal and lung inflammation caused by basophil activation and effectively reduce or eliminate symptoms of asthma, emphysema, and allergic rhinitis.

AX. CG56258-02: Sodium/Calcium Exchanger.

Expression of gene CG56258-02 was assessed using the primer-probe sets Ag2903, Ag5035 and Ag6163, described in Tables AXA, AXB and AXC. Results of the RTQ-PCR runs are shown in Tables AXD, AXE, AXF, AXG, AXH, AXI and AXJ.

TABLE AXA
Probe Name Ag2903

| | | | Start | SEQ ID |
|---|---|---|---|---|
| Primers | Sequences | Length | Position | No |
| Forward | 5′-gactcgcaagatcaagcatcta-3′ | 22 | 641 | 1389 |
| Probe | TET-5′-cttcttcatcaccgctgcttggagta-3′- | 26 | 668 | 1390 |
| | TAMRA | | | |
| Reverse | 5′-tagagccagatgtaggcaaaga-3′ | 22 | 694 | 1391 |

TABLE AXB
Probe Name Ag5035

| | | | Start | SEQ ID |
|---|---|---|---|---|
| Primers | Sequences | Length | Position | No |
| Forward | 5′-gaaagccagtattgggtgaac-3′ | 21 | 2023 | 1392 |
| Probe | TET-5′-ccccaaactagaagtcatcattgaaga- | 27 | 2045 | 1393 |
| | 3′-TAMRA | | | |
| Reverse | 5′-tttgtccaccgtagtcttgaac-3′ | 22 | 2081 | 1394 |

TABLE AXC
Probe Name Ag6163

| | | | Start | SEQ ID |
|---|---|---|---|---|
| Primers | Sequences | Length | Position | No |
| Forward | 5′-ggggagttggaattcaagaat-3′ | 21 | 1815 | 1395 |
| Probe | TET-5′-tgaaactgtcaaaacaattcacatcaag- | 28 | 1838 | 1396 |
| | 3′-TAMRA | | | |
| Reverse | 5′-tctcatatgcctcatcatcaattac-3′ | 25 | 1866 | 1397 |

TABLE AXD
AI_comprehensive panel_v1.0
Column A - Rel. Exp.(%) Ag2903, Run 225410015
Column B - Rel. Exp.(%) Ag5035, Run 244570389

| Tissue Name | A | B | Tissue Name | A | B |
|---|---|---|---|---|---|
| 110967 COPD-F | 0.6 | 0.3 | 112427 Match Control Psoriasis-F | 4.1 | 4.8 |
| 110980 COPD-F | 0.4 | 0.6 | 112418 Psoriasis-M | 0.8 | 0.5 |
| 110968 COPD-M | 0.9 | 1.2 | 112723 Match Control Psoriasis-M | 0.1 | 0.0 |
| 110977 COPD-M | 0.9 | 0.8 | 112419 Psoriasis-M | 1.3 | 0.9 |
| 110989 Emphysema-F | 0.6 | 0.0 | 112424 Match Control Psoriasis-M | 1.1 | 0.5 |
| 110992 Emphysema-F | 0.4 | 0.7 | 112420 Psoriasis-M | 2.1 | 2.2 |
| 110993 Emphysema-F | 1.1 | 1.4 | 112425 Match Control Psoriasis-M | 2.9 | 5.8 |
| 110994 Emphysema-F | 1.0 | 0.7 | 104689 (MF) OA Bone-Backus | 68.3 | 44.4 |
| 110995 Emphysema-F | 1.1 | 0.8 | 104690 (ME) Adj “Normal” Bone- | 8.8 | 5.2 |
| | | | Backus | | |
| 110996 Emphysema-F | 0.0 | 0.0 | 104691 (ME) OA Synovium- | 2.1 | 1.7 |
| | | | Backus | | |
| 110997 Asthma-M | 1.5 | 0.6 | 104692 (BA) OA Cartilage-Backus | 3.3 | 3.6 |
| 111001 Asthma-F | 1.8 | 1.5 | 104694 (BA) OA Bone-Backus | 100.0 | 100.0 |
| 111002 Asthma-F | 1.9 | 1.9 | 104695 (BA) Adj “Normal” Bone- | 36.3 | 28.7 |
| | | | Backus | | |
| 111003 Atopic Asthma-F | 2.7 | 2.4 | 104696 (BA) OA Synovium- | 1.4 | 0.5 |
| | | | Backus | | |
| 111004 Atopic Asthma-F | 0.9 | 1.2 | 104700 (SS) OA Bone-Backus | 54.0 | 37.6 |
| 111005 Atopic Asthma-F | 0.9 | 1.1 | 104701 (SS) Adj “Normal” Bone- | 60.3 | 34.9 |
| | | | Backus | | |
| 111006 Atopic Asthma-F | 0.4 | 0.3 | 104702 (SS) OA Synovium-Backus | 2.9 | 2.1 |
| 111417 Allergy-M | 2.2 | 2.8 | 117093 OA Cartilage Rep7 | 1.4 | 0.4 |
| 112347 Allergy-M | 0.6 | 0.0 | 112672 OA Bone5 | 4.8 | 3.3 |
| 112349 Normal Lung-F | 0.9 | 0.0 | 112673 OA Synovium5 | 1.6 | 1.7 |
| 112357 Normal Lung-F | 0.1 | 0.3 | 112674 OA Synovial Fluid cells5 | 2.6 | 2.6 |
| 112354 Normal Lung-M | 0.0 | 0.3 | 117100 OA Cartilage Rep14 | 0.0 | 0.0 |
| 112374 Crohns-F | 0.2 | 0.0 | 112756 OA Bone9 | 5.6 | 0.4 |
| 112389 Match Control Crohns- | 0.2 | 1.1 | 112757 OA Synovium9 | 32.3 | 37.4 |
| F | | | | | |
| 112375 Crobns-F | 0.0 | 0.0 | 112758 OA Synovial Fluid Cells9 | 1.1 | 0.6 |
| 112732 Match Control Crohns- | 0.8 | 0.8 | 117125 RA Cartilage Rep2 | 2.8 | 1.1 |
| F | | | | | |
| 112725 Crohns-M | 0.1 | 0.0 | 113492 Bone2 RA | 3.0 | 1.2 |
| 112387 Match Control Crohns- | 1.6 | 1.1 | 113493 Synovium2 RA | 1.6 | 0.8 |
| M | | | | | |
| 112378 Crohns-M | 0.6 | 0.0 | 113494 Syn Fluid Cells RA | 1.8 | 0.8 |
| 112390 Match Control Crohns- | 1.0 | 0.8 | 113499 Cartilage4 RA | 1.7 | 1.9 |
| M | | | | | |
| 112726 Crohns-M | 0.8 | 0.7 | 113500 Bone4 RA | 2.6 | 2.2 |
| 112731 Match Control Crohns- | 0.9 | 0.3 | 113501 Synovium4 RA | 2.0 | 0.7 |
| M | | | | | |
| 112380 Ulcer Col-F | 0.4 | 0.5 | 113502 Syn Fluid Cells4 RA | 0.6 | 0.6 |
| 112734 Match Control Ulcer | 3.5 | 1.8 | 113495 Cartilage3 RA | 1.6 | 0.6 |
| Col-F | | | | | |
| 112384 Ulcer Col-F | 2.7 | 1.9 | 113496 Bone3 RA | 1.9 | 0.7 |
| 112737 Match Control Ulcer | 0.5 | 0.6 | 113497 Synovium3 RA | 1.4 | 0.8 |
| Col-F | | | | | |
| 112386 Ulcer Col-F | 2.0 | 1.4 | 113498 Syn Fluid Cells3 RA | 2.6 | 2.7 |
| 112738 Match Control Ulcer | 0.1 | 0.3 | 117106 Normal Cartilage Rep20 | 0.4 | 0.0 |
| Col-F | | | | | |
| 112381 Ulcer Col-M | 1.3 | 0.0 | 113663 Bone3 Normal | 0.6 | 0.0 |
| 112735 Match Control Ulcer | 3.3 | 1.2 | 113664 Synovium3 Normal | 0.2 | 0.0 |
| Col-M | | | | | |
| 112382 Ulcer Col-M | 1.2 | 0.6 | 113665 Syn Fluid Cells3 Normal | 0.2 | 0.0 |
| 112394 Match Control Ulcer | 0.9 | 0.8 | 117107 Normal Cartilage Rep22 | 2.9 | 0.4 |
| Col-M | | | | | |
| 112383 Ulcer Col-M | 0.7 | 0.0 | 113667 Bone4 Normal | 1.1 | 0.0 |
| 112736 Match Control Ulcer | 0.7 | 0.3 | 113668 Synovium4 Normal | 1.3 | 0.5 |
| Col-M | | | | | |
| 112423 Psoriasis-F | 0.7 | 0.3 | 113669 Syn Fluid Cells4 Normal | 1.1 | 0.5 |

TABLE AXE
Cellular OA/RA
Column A - Rel. Exp.(%) Ag2903, Run 406107093

| Tissue Name | A | Tissue Name | A |
|---|---|---|---|
| 158667 Nhost medium 1 h | 0.0 | 164336 SW1353 + TNF-a (100 ng/ml) 6 h | 54.3 |
| 158670 Nhost + IL-1b (10 | 4.2 | 164337 SW1353 medium alone 18 h | 74.2 |
| ng/ml), 1 h | | | |
| 158673 Nhost + PGE2 (10-6 M) | 0.0 | 164338 SW1353 + IL-1b (1 ng/ml) 18 h | 62.9 |
| 1 h | | | |
| 158668 Nhost medium alone 6 h | 2.3 | 164339 SW1353 + IL-1b (10 ng/ml) 18 h | 41.5 |
| 158671 Nhost + IL-1b (10 | 29.1 | 164340 SW1353 + TNF-a (10 ng/ml) 18 h | 49.3 |
| ng/ml) 6 h | | | |
| 158674 Nhost + PGE2 (10-6 M) | 2.5 | 164341 SW1353 + IL-1b (100 ng/ml) 18 h | 74.2 |
| 6 h | | | |
| 158669 Nhost medium alone 24 h | 1.4 | 173326 HFLS-RA (cell aplication) medium | 0.0 |
| | | alone 18 h | |
| 158672 Nhost + IL-1b (10 | 5.4 | 173327 HFLS-RA (cell aplication) + TNF-a | 12.9 |
| ng/ml) 24 h | | 18 h | |
| 158675 Nhost + PGE2 (10-6 M) | 1.2 | 173331 MH7A (synoviocyte cell line) | 46.7 |
| 24 h | | medium 1 h | |
| 164327 SW1353 medium alone | 95.3 | 173332 MH7A (synoviocyte cell line) + | 29.1 |
| 1 h | | IL1b 1 h | |
| 164328 SW1353 + IL-1b (1 | 88.3 | 173334 MH7A (synoviocyte cell line) TNFa | 0.0 |
| ng/ml) 1 h | | 1 h | |
| 164329 SW1353 + IL-1b (10 | 68.8 | 173336 MH7A (synoviocyte cell line) | 63.7 |
| ng/ml) 1 h | | medium alone 6 h | |
| 164330 SW1353 + TNF-a (10 | 82.9 | 173339 MH7A (synoviocyte cell line) + | 61.6 |
| ng/ml) 1 h | | IL1b 6 h | |
| 164331 SW1353 + TNF-a (100 | 100.0 | 173341 MH7A (synoviocyte cell line) TNFa | 37.9 |
| ng/ml) 1 h | | 6 h | |
| 164332 SW1353 medium alone | 54.7 | 173342 MH7A (synoviocyte cell line) | 37.4 |
| 6 h | | medium alone 18 h | |
| 164333 SW1353 + IL-1b (1 | 41.5 | 173344 MH7A (synoviocyte cell line) + | 73.7 |
| ng/ml) 6 h | | IL1b 18 h | |
| 164334 SW1353 + IL-1b (10 | 29.1 | 173346 MH7A (synoviocyte cell line) TNF-a | 32.1 |
| ng/ml) 6 h | | 18 h | |
| 164335 SW1353 + TNF-a (10 | 88.3 | | |
| ng/ml) 6 h | | | |

TABLE AXF
PGI1.0
Column A - Rel. Exp.(%) Ag2903, Run 398125347

| Tissue Name | A | Tissue Name | A |
|---|---|---|---|
| 162191 Normal Lung 1 (IBS) | 1.4 | 162185 Emphysema Lung 12 (Ardais) | 41.5 |
| 160468 MD lung | 13.7 | 162184 Emphysema Lung 13 (Ardais) | 23.2 |
| 156629 MD Lung 13 | 5.8 | 162183 Emphysema Lung 14 (Ardais) | 70.7 |
| 162570 Normal Lung 4 (Aastrand) | 18.6 | 162188 Emphysema Lung 15 (Genomic | 51.4 |
| | | Collaborative) | |
| 162571 Normal Lung 3 (Aastrand) | 6.1 | 162177 NAT UC Colon 1 (Ardais) | 7.6 |
| 162187 Fibrosis Lung 2 (Genomic | 100.0 | 162176 UC Colon 1 (Ardais) | 7.7 |
| Collaborative) | | | |
| 151281 Fibrosis lung 11 (Ardais) | 25.7 | 162179 NAT UC Colon 2 (Ardais) | 11.0 |
| 162186 Fibrosis Lung 1 (Genomic | 67.8 | 162178 UC Colon 2 (Ardais) | 19.5 |
| Collaborative) | | | |
| 162190 Asthma Lung 4 (Genomic | 38.4 | 162181 NAT UC Colon 3 (Ardais) | 3.1 |
| Collaborative) | | | |
| 160467 Asthma Lung 13 (MD) | 10.9 | 162180 UC Colon 3 (Ardais) | 2.9 |
| 137027 Emphysema Lung 1 | 13.8 | 162182 NAT UC Colon 4 (Ardais) | 6.5 |
| (Ardais) | | | |
| 137028 Emphysema Lung 2 | 11.4 | 137042 UC Colon 1108 | 8.0 |
| (Ardais) | | | |
| 137040 Emphysema Lung 3 | 14.8 | 137029 UC Colon 8215 | 5.2 |
| (Ardais) | | | |
| 137041 Emphysema Lung 4 | 23.3 | 137031 UC Colon 8217 | 4.9 |
| (Ardais) | | | |
| 137043 Emphysema Lung 5 | 12.9 | 137036 UC Colon 1137 | 9.2 |
| (Ardais) | | | |
| l428l7 Emphysema Lung 6 | 17.3 | 137038 UC Colon 1491 | 27.2 |
| (Ardais) | | | |
| 142818 Emphysema Lung 7 | 36.6 | 137039 UC Colon 1546 | 12.3 |
| (Ardais) | | | |
| 142819 Emphysema Lung 8 | 36.1 | 162593 Crohn's 47751 (NDRI) | 3.5 |
| (Ardais) | | | |
| 142820 Emphysema Lung 9 | 18.0 | 162594 NAT Crohn's 47751 (NDRI) | 3.1 |
| (Ardais) | | | |
| 142821 Emphysema Lung 10 | 49.3 | | |
| (Ardais) | | | |

TABLE AXG
Panel 1.3D
Column A - Rel. Exp.(%) Ag2903, Run 162556420

| Tissue Name | A | Tissue Name | A |
|---|---|---|---|
| Liver adenocarcinoma | 0.0 | Kidney (fetal) | 0.3 |
| Pancreas | 0.0 | Renal ca. 786-0 | 0.0 |
| Pancreatic ca. CAPAN 2 | 0.0 | Renal ca. A498 | 0.0 |
| Adrenal gland | 0.1 | Renal ca. RXF 393 | 0.0 |
| Thyroid | 0.2 | Renal ca. ACHN | 0.0 |
| Salivary gland | 0.1 | Renal ca. UO-31 | 0.1 |
| Pituitary gland | 0.4 | Renal ca. TK-10 | 0.0 |
| Brain (fetal) | 2.0 | Liver | 0.0 |
| Brain (whole) | 3.9 | Liver (fetal) | 0.4 |
| Brain (amygdala) | 3.7 | Liver ca. (hepatoblast) HepG2 | 0.0 |
| Brain (cerebellum) | 3.3 | Lung | 0.0 |
| Brain (hippocampus) | 5.6 | Lung (fetal) | 0.4 |
| Brain (substantia nigra) | 0.9 | Lung ca. (small cell) LX-1 | 0.0 |
| Brain (thalamus) | 5.9 | Lung ca. (small cell) NCI-H69 | 0.2 |
| Cerebral Cortex | 80.7 | Lung ca. (s. cell var.) SHP-77 | 3.4 |
| Spinal cord | 1.7 | Lung ca. (large cell) NCI-H460 | 0.0 |
| glio/astro U87-MG | 0.0 | Lung ca. (non-sm. cell) A549 | 0.1 |
| glio/astro U-118-MG | 0.0 | Lung ca. (non-s. cell) NCI-H23 | 0.0 |
| astrocytoma SW1783 | 0.6 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.2 |
| glioma SNB-19 | 0.1 | Mammary gland | 0.1 |
| glioma U251 | 0.0 | Breast ca.* (pl. ef) MCF-7 | 0.0 |
| glioma SF-295 | 0.0 | Breast ca.* (pl. ef) MDA-MB-231 | 0.0 |
| Heart (Fetal) | 5.2 | Breast ca.* (pl. ef) T47D | 0.0 |
| Heart | 0.3 | Breast ca. BT-549 | 0.0 |
| Skeletal muscle (Fetal) | 100.0 | Breast ca. MDA-N | 0.0 |
| Skeletal muscle | 21.2 | Ovary | 0.2 |
| Bone marrow | 0.2 | Ovarian ca. OVCAR-3 | 0.0 |
| Thymus | 0.6 | Ovarian ca. OVCAR-4 | 0.3 |
| Spleen | 0.0 | Ovarian ca. OVCAR-5 | 0.0 |
| Lymph node | 0.3 | Ovarian ca. OVCAR-8 | 0.0 |
| Colorectal | 1.1 | Ovarian ca. IGROV-1 | 0.0 |
| Stomach | 0.1 | Ovarian ca. (ascites) SK-OV-3 | 0.0 |
| Small intestine | 0.2 | Uterus | 0.1 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone met) PC-3 | 0.0 |
| Colon ca. HCT-116 | 0.0 | Testis | 0.3 |
| Colon ca. CaCo-2 | 0.0 | Melanoma Hs688(A).T | 0.0 |
| CC Well to Mod Diff | 0.5 | Melanoma* (met) Hs688(B).T | 0.0 |
| (ODO3866) | | | |
| Colon ca. HCC-2998 | 0.0 | Melanoma UACC-62 | 0.2 |
| Gastric ca. (liver met) | 0.0 | Melanoma M14 | 0.0 |
| NCI-N87 | | | |
| Bladder | 0.2 | Melanoma LOXIMVI | 0.0 |
| Trachea | 0.3 | Melanoma* (met) SK-MEL-5 | 0.0 |
| Kidney | 0.0 | Adipose | 0.3 |

TABLE AXH
Panel 2D
Column A - Rel. Exp.(%) Ag2903, Run 162345106

| Tissue Name | A | Tissue Name | A |
|---|---|---|---|
| Normal Colon | 8.1 | Kidney Margin 8120608 | 0.5 |
| CC Well to Mod Diff (ODO3866) | 0.3 | Kidney Cancer 8120613 | 0.0 |
| CC Margin (ODO3866) | 0.3 | Kidney Margin 8120614 | 0.1 |
| CC Gr.2 rectosigmoid (ODO3868) | 0.1 | Kidney Cancer 9010320 | 0.5 |
| CC Margin (ODO3868) | 0.2 | Kidney Margin 9010321 | 0.0 |
| CC Mod Diff(ODO3920) | 0.3 | Normal Uterus | 1.0 |
| CC Margin (ODO3920) | 0.4 | Uterine Cancer 064011 | 0.5 |
| CC Gr.2 ascend colon (ODO3921) | 1.1 | Normal Thyroid | 1.0 |
| CC Margin (ODO3921) | 0.9 | Thyroid Cancer | 0.0 |
| CC from Partial Hepatectomy | 0.4 | Thyroid Cancer A302152 | 0.1 |
| (ODO4309) Mets | | | |
| Liver Margin (ODO4309) | 0.1 | Thyroid Margin A302153 | 0.1 |
| Colon mets to lung (OD04451-01) | 0.1 | Normal Breast | 3.0 |
| Lung Margin (OD04451-02) | 1.3 | Breast Cancer | 0.4 |
| Normal Prostate 6546-1 | 2.3 | Breast Cancer (OD04590-01) | 0.8 |
| Prostate Cancer (OD04410) | 1.2 | Breast Cancer Mets (OD04590-03) | 1.5 |
| Prostate Margin (OD04410) | 4.2 | Breast Cancer Metastasis | 0.2 |
| Prostate Cancer (OD04720-01) | 1.2 | Breast Cancer | 0.5 |
| Prostate Margin (OD04720-02) | 4.6 | Breast Cancer | 1.2 |
| Normal Lung | 5.8 | Breast Cancer 9100266 | 1.8 |
| Lung Met to Muscle (ODO4286) | 0.0 | Breast Margin 9100265 | 1.1 |
| Muscle Margin (ODO4286) | 100.0 | Breast Cancer A209073 | 0.6 |
| Lung Malignant Cancer (OD03126) | 0.8 | Breast Margin A209073 | 0.0 |
| Lung Margin (OD03126) | 7.7 | Normal Liver | 0.0 |
| Lung Cancer (OD04404) | 1.4 | Liver Cancer | 0.0 |
| Lung Margin (OD04404) | 4.1 | Liver Cancer 1025 | 0.0 |
| Lung Cancer (OD04565) | 0.3 | Liver Cancer 1026 | 0.0 |
| Lung Margin (OD04565) | 1.2 | Liver Cancer 6004-T | 0.1 |
| Lung Cancer (OD04237-01) | 0.7 | Liver Tissue 6004-N | 0.5 |
| Lung Margin (OD04237-02) | 2.4 | Liver Cancer 6005-T | 0.0 |
| Ocular Mel Met to Liver (ODO4310) | 0.0 | Liver Tissue 6005-N | 0.0 |
| Liver Margin (ODO4310) | 0.1 | Normal Bladder | 0.6 |
| Melanoma Metastasis | 0.1 | Bladder Cancer | 0.2 |
| Lung Margin (OD04321) | 2.4 | Bladder Cancer | 1.4 |
| Normal Kidney | 1.5 | Bladder Cancer (OD04718-01) | 0.5 |
| Kidney Ca, Nuclear grade 2 (OD04338) | 0.5 | Bladder Normal Adjacent | 4.9 |
| | | (OD04718-03) | |
| Kidney Margin (OD04338) | 1.4 | Normal Ovary | 0.1 |
| Kidney Ca Nuclear grade 1/2 (OD04339) | 1.8 | Ovarian Cancer | 1.9 |
| Kidney Margin (OD04339) | 0.6 | Ovarian Cancer (OD04768-07) | 0.0 |
| Kidney Ca, Clear cell type (OD04340) | 1.0 | Ovary Margin (OD04768-08) | 0.2 |
| Kidney Margin (OD04340) | 0.8 | Normal Stomach | 4.5 |
| Kidney Ca, Nuclear grade 3 (OD04348) | 1.3 | Gastric Cancer 9060358 | 1.7 |
| Kidney Margin (OD04348) | 0.7 | Stomach Margin 9060359 | 1.5 |
| Kidney Cancer (OD04622-01) | 0.7 | Gastric Cancer 9060395 | 1.2 |
| Kidney Margin (OD04622-03) | 0.0 | Stomach Margin 9060394 | 2.0 |
| Kidney Cancer (OD04450-01) | 0.0 | Gastric Cancer 9060397 | 0.6 |
| Kidney Margin (OD04450-03) | 0.1 | Stomach Margin 9060396 | 1.7 |
| Kidney Cancer 8120607 | 0.1 | Gastric Cancer 064005 | 2.9 |

TABLE AXI
Panel 4.1D
Column A - Rel. Exp.(%) Ag5035, Run 223740981

| Tissue Name | A | Tissue Name | A |
|---|---|---|---|
| Secondary Th1 act | 0.0 | HUVEC IL-1beta | 2.0 |
| Secondary Th2 act | 0.0 | HUVEC IFN gamma | 0.0 |
| Secondary Tr1 act | 0.0 | HUVEC TNF alpha + IFN gamma | 1.9 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 100.0 |
| | | 1beta | |
| Primary Th2 act | 0.0 | Microvascular Dermal EC none | 0.0 |
| Primary Tr1 act | 2.0 | Microsvasular Dermal EC TNFalpha + IL- | 55.5 |
| | | 1beta | |
| Primary Th1 rest | 0.0 | Bronchial epithelium TNFalpha +IL1beta | 2.2 |
| Primary Th2 rest | 0.0 | Small airway epithelium none | 4.2 |
| Primary Tr1 rest | 0.0 | Small airway epithelium TNFalpha + IL- | 3.7 |
| | | 1beta | |
| CD45RA CD4 lymphocyte act | 0.0 | Coronery artery SMC rest | 2.3 |
| CD45RO CD4 lymphocyte act | 0.0 | Coronery artery SMC TNFalpha + IL-1beta | 0.0 |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 10.6 |
| CD4 lymphocyte none | 0.0 | KU-812 (Basophil) PMA/ionomycin | 13.6 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 0.0 |
| LAK cells IL-2 + IL-12 | 8.4 | NCI-H292 none | 0.0 |
| LAK cells IL-2 + IFN gamma | 2.9 | NCI-H292 IL-4 | 0.0 |
| LAK cells IL-2 + IL-18 | 3.8 | NCI-H292 IL-9 | 0.0 |
| LAK cells PMA/ionomycin | 2.3 | NCI-H292 IL-13 | 2.5 |
| NK Cells IL-2 rest | 0.0 | NCI-H292 IFN gamma | 0.0 |
| Two Way MLR 3 day | 2.1 | HPAEC none | 0.0 |
| Two Way MLR 5 day | 2.3 | HPAEC TNF alpha + IL-1 beta | 8.8 |
| Two Way MLR 7 day | 1.8 | Lung fibroblast none | 0.0 |
| PBMC rest | 6.7 | Lung fibroblast TNF alpha + IL-1 beta | 0.0 |
| PBMC PWM | 0.0 | Lung fibroblast IL-4 | 0.0 |
| PBMC PHA-L | 6.3 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 9.2 | Dermal fibroblast CCD1070 rest | 0.0 |
| B lymphocytes CD40L and IL-4 | 1.9 | Dermal fibroblast CCD1070 TNF alpha | 0.0 |
| EOL-1 dbcAMP | 0.0 | Dermal fibroblast CCD1070 IL-1 beta | 0.0 |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 2.4 | Dermal Fibroblasts rest | 2.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 31.6 | Colon | 5.4 |
| Macrophages rest | 1.7 | Lung | 8.1 |
| Macrophages LPS | 6.5 | Thymus | 5.1 |
| HUVEC none | 0.0 | Kidney | 0.0 |
| HUVEC starved | 0.0 | | |

TABLE AXJ
Panel 5 Islet
Column A - Rel. Exp.(%) Ag2903, Run 242321748
Column B - Rel. Exp.(%) Ag2903, Run 258900739
Column C - Rel. Exp.(%) Ag5035, Run 244908253

| Tissue Name | A | B | C | Tissue Name | A | B | C |
|---|---|---|---|---|---|---|---|
| 97457 Patient-02go adipose | 0.7 | 0.8 | 1.4 | 94709 Donor 2 AM - A | 0.4 | 0.4 | 0.0 |
| | | | | adipose | | | |
| 97476 Patient-07sk skeletal | 9.9 | 15.2 | 3.2 | 94710 Donor 2 AM - B | 0.9 | 1.2 | 0.0 |
| muscle | | | | adipose | | | |
| 97477 Patient-07ut uterus | 0.6 | 1.4 | 0.0 | 94711 Donor2 AM - C | 0.0 | 0.4 | 0.0 |
| | | | | adipose | | | |
| 97478 Patient-07pl placenta | 1.4 | 0.0 | 1.1 | 94712 Donor 2 AD - A | 0.0 | 0.0 | 0.0 |
| | | | | adipose | | | |
| 99167 Bayer Patient 1 | 26.6 | 26.4 | 24.8 | 94713 Donor2 AD - B | 0.4 | 0.3 | 0.0 |
| | | | | adipose | | | |
| 97482 Patient-08ut uterus | 0.0 | 0.3 | 0.0 | 94714 Donor 2 AD - C | 0.0 | 0.8 | 0.0 |
| | | | | adipose | | | |
| 97483 Patient-08pl placenta | 0.0 | 0.7 | 0.0 | 94742 Donor 3 U - A | 0.0 | 0.0 | 0.0 |
| | | | | Mesenchymal Stem Cells | | | |
| 97486 Patient-09sk skeletal | 10.4 | 19.5 | 11.1 | 94743 Donor 3 U - B | 0.9 | 0.0 | 0.0 |
| muscle | | | | Mesenchymal Stem Cells | | | |
| 97487 Patient-09ut uterus | 0.6 | 2.8 | 1.7 | 94730 Donor 3 AM - A | 0.9 | 0.3 | 0.0 |
| | | | | adipose | | | |
| 97488 Patient-09pl placenta | 0.3 | 1.1 | 1.1 | 94731 Donor 3 AM - B | 0.0 | 0.0 | 0.0 |
| | | | | adipose | | | |
| 97492 Patient-10ut uterus | 0.2 | 0.4 | 0.0 | 94732 Donor 3 AM - C | 0.0 | 0.7 | 0.0 |
| | | | | adipose | | | |
| 97493 Patient-10pl placenta | 0.8 | 0.4 | 1.1 | 94733 Donor 3 AD - A | 1.0 | 0.3 | 0.0 |
| | | | | adipose | | | |
| 97495 Patient-11go adipose | 1.4 | 3.1 | 0.0 | 94734 Donor 3 AD - B | 0.0 | 0.4 | 0.0 |
| | | | | adipose | | | |
| 97496 Patient-11sk skeletal | 29.1 | 59.9 | 33.9 | 94735 Donor 3 AD - C | 0.0 | 0.0 | 0.0 |
| muscle | | | | adipose | | | |
| 97497 Patient-11ut uterus | 0.5 | 0.4 | 0.0 | 77138 Liver HepG2untreated | 0.0 | 0.4 | 0.0 |
| 97498 Patient-11pl placenta | 0.0 | 0.0 | 0.0 | 73556 Heart Cardiac stromal | 0.5 | 0.4 | 0.0 |
| | | | | cells (primary) | | | |
| 97500 Patient-12go adipose | 0.5 | 2.8 | 0.0 | 81735 Small Intestine | 2.3 | 0.7 | 0.0 |
| 97501 Patient-12sk skeletal | 100.0 | 100.0 | 100.0 | 72409 Kidney Proximal | 0.0 | 0.4 | 0.0 |
| muscle | | | | Convoluted Tubule | | | |
| 97502 Patient-12ut uterus | 1.0 | 0.3 | 0.0 | 82685 Small intestine | 0.0 | 1.7 | 1.1 |
| | | | | Duodenum | | | |
| 97503 Patient-12pl placenta | 0.0 | 0.0 | 0.0 | 90650 Adrenal | 0.0 | 0.0 | 0.0 |
| | | | | Adrenocortical adenoma | | | |
| 94721 Donor 2 U - A | 0.4 | 0.0 | 0.0 | 72410 Kidney HRCE | 0.0 | 0.0 | 1.6 |
| Mesenchymal Stem Cells | | | | | | | |
| 94722 Donor 2 U - B | 0.4 | 1.4 | 0.0 | 72411 Kidney HRE | 0.4 | 1.1 | 0.0 |
| Mesenchymal Stem Cells | | | | | | | |
| 94723 Donor 2 U - C | 0.0 | 0.0 | 0.0 | 73139 Uterus Uterine smooth | 0.0 | 0.0 | 0.0 |
| Mesenchymal Stem Cells | | | | muscle cells | | | |

AI_comprehensive panel_v1.0 Summary: Ag2903/Ag5035 The highest expression of this gene was detected in an OA bone sample. Expression of the CG56258-01 gene was highly associated with synovium and bone samples from patients with osteoarthritis when compared to expression in the control samples. Therapeutic modulation of this gene, expressed protein and/or use of antibodies or small molecule drugs targeting the gene or gene product are useful in the treatment of osteoarthritis.

Cellular OA/RA Summary: Ag2903 Moderate expression of this gene was detected in chondrosarcoma cell line (SW1353) and synoviocyte cell line (CTs=31-32). Significant expression of this gene was also detected in cells treated with IL-1-beta, a potent activator of pro-inflammatory cytokines and matrix metalloproteinases which participate in the destruction of cartilage observed in Osteoarthritis (OA). Therapeutic modulation of this gene, expressed protein and/or use of antibodies or small molecule drugs targeting the gene or gene product are useful in the prevention or treatment of the degeneration of cartilage observed in OA.

PGI1.0 Summary: Ag2903 The highest expression level of this gene was detected in a lung fibrosis sample (CT=26). It was upregulated in lung fibrosis and several emphysema samples. Therapeutic modulation of this gene, expressed protein and/or use of antibodies or small molecule drugs targeting the gene or gene product are useful in the treatment of lung fibrosis and emphysema.

Panel 1.3D Summary: Ag2903 Expression of this gene was highest in fetal skeletal muscle (CT=26.8). Significant levels of expression are also seen in adult skeletal muscle and fetal heart. This gene encodes a putative sodium/calcium exchanger. Altered levels of intracellular calcium have been implicated in many diseases, including type 2 diabetes. Based on its expression profile and homology to a calcium transport protein, therapeutic modulation of this gene, expressed protein and/or use of antibodies or small molecule drugs targeting the gene or gene product are useful in the treatment of type 2 diabetes.

Moderate to low levels of expression were seen in all regions of the CNS examined. Inhibition of calcium uptake has been shown to decrease neuronal death in response to cerebral ischemia. Therapeutic modulation of this gene, expressed protein and/or use of antibodies or small molecule drugs targeting the gene or gene product are useful in the treatment of stroke by decreasing the total infarct volume.

Panel 2D Summary: Ag2903 The expression of the CG56258-01 gene was consistent with the profile seen in Panel 1.3D. Expression was highest and most prominent in a normal muscle sample (CT=28.7). Please see Panel 1.3D for discussion of this gene in metabolic disease.

Panel 4.1D Summary: Ag5035 Expression of the CG56258-02 gene was restricted to TNF-alpha and IL-1 beta treated lung and dermal microvasculature (CTs=33-34). Endothelial cells are known to play important roles in inflammatory responses by altering the expression of surface proteins that are involved in activation and recruitment of effector inflammatory cells. The expression of this gene in dermal microvascular endothelial cells indicated that this protein product is involved in inflammatory responses to skin disorders, including psoriasis. Expression in lung microvascular endothelial cells indicated that the protein encoded by this gene is involved in lung disorders including asthma, allergies, chronic obstructive pulmonary disease, and emphysema. Therapeutic modulation of this gene, expressed protein and/or use of antibodies or small molecule drugs targeting the gene or gene product are useful in the treatment of psoriasis, asthma, allergies, chronic obstructive pulmonary disease, and emphysema.

Panel 5 Islet Summary: Ag2903/Ag5035 The expression of this gene in this panel was consistent with the profile seen in Panel 1.3D. Expression was highest and most prominent in sampels derived from skeletal muscle (CTs=29-33). Please see Panel 1.3D for discussion of this gene in metabolic disease.

AY. CG56258-04: SCL8A3 Splice Form B-Like.

Expression of gene CG56258-04 was assessed using the primer-probe sets Ag5035 and Ag6142, described in Tables AYA and AYB. Results of the RTQ-PCR runs are shown in Tables AYC, AYD, AYE and AYF.

TABLE AYA
Probe Name Ag5035

| | | | Start | SEQ ID |
|---|---|---|---|---|
| Primers | Sequences | Length | Position | No |
| Forward | 5′-gaaagccagtattgggtgaac-3′ | 21 | 2011 | 1398 |
| Probe | TET-5′-ccccaaactagaagtcatcattgaaga- | 27 | 2033 | 1399 |
| | 3′-TAMRA | | | |
| Reverse | 5′-tttgtccaccgtagtcttgaac-3′ | 22 | 2069 | 1340 |

TABLE AYB
Probe Name Ag6142

| | | | Start | SEQ ID |
|---|---|---|---|---|
| Primers | Sequences | Length | Position | No |
| Forward | 5′-gtagatgaggaggaatacgaaagg-3′ | 24 | 1869 | 1341 |
| Probe | TET-5′-aatttcttcattgcccttggtgaacc-3′- | 26 | 1899 | 1342 |
| | TAMRA | | | |
| Reverse | 5′-gatattccacgttccatccatt-3′ | 22 | 1927 | 1343 |

TABLE AYC
AI_comprehensive panel_v1.0
Column A - Rel. Exp.(%) Ag5035, Run 244570389
Column B - Rel. Exp.(%) Ag6142, Run 253050665

| | | | | | |
|---|---|---|---|---|---|
| Tissue Name | A | B | Tissue Name | A | B |

| | | | | | |
|---|---|---|---|---|---|
| 110967 COPD-F | 0.3 | 1.5 | 112427 Match Control Psoriasis-F | 4.8 | 9.8 |
| 110980 COPD-F | 0.6 | 0.0 | 112418 Psoriasis-M | 0.5 | 1.0 |
| 110968 COPD-M | 1.2 | 0.8 | 112723 Match Control Psoriasis-M | 0.0 | 0.0 |
| 110977 COPD-M | 0.8 | 1.3 | 112419 Psoriasis-M | 0.9 | 1.7 |
| 110989 Emphysema-F | 0.0 | 0.6 | 112424 Match Control Psoriasis-M | 0.5 | 0.8 |
| 110992 Emphysema-F | 0.7 | 0.7 | 112420 Psoriasis-M | 2.2 | 1.6 |
| 110993 Emphysema-F | 1.4 | 0.0 | 112425 Match Control Psoriasis-M | 5.8 | 4.4 |
| 110994 Emphysema-F | 0.7 | 0.6 | 104689 (MF) OA Bone-Backus | 44.4 | 52.9 |
| 110995 Emphysema-F | 0.8 | 0.7 | 104690 (MF) Adj “Normal” Bone | | |
| | | | Backus | 5.2 | 2.8 |
| 110996 Emphysema-F | 0.0 | 0.0 | 104691 (MF) OA Synovium- | | |
| | | | Backus | 1.7 | 1.5 |
| 110997 Asthma-M | 0.6 | 1.2 | 104692 (BA) OA Cartilage-Backus | 3.6 | 3.0 |
| 111001 Asthma-F | 1.5 | 0.8 | 104694 (BA) OA Bone-Backus | 100.0 | 100.0 |
| 111002 Asthma-F | 1.9 | 0.0 | 104695 (BA) Adj “Normal” Bone- | 28.7 | 33.7 |
| | | | Backus | | |
| 111003 Atopic Asthma-F | 2.4 | 1.9 | 104696 (BA) OA Synovium- | 0.5 | 0.7 |
| | | | Backus | | |
| 111004 Atopic Asthma-F | 1.2 | 0.8 | 104700 (55) OA Bone-Backus | 37.6 | 28.9 |
| 111005 Atopic Asthma-F | 1.1 | 1.5 | 104701 (SS) Adj “Normal” Bone- | 34.9 | 27.5 |
| | | | Backus | | |
| 111006 Atopic Asthma-F | 0.3 | 0.0 | 104702 (SS) OA Synovium-Backus | 2.1 | 2.5 |
| 111417 Allergy-M | 2.8 | 0.0 | 117093 OA Cartilage Rep7 | 0.41 | 0.6 |
| 112347 Allergy-M | 0.0 | 0.0 | 112672 OA Bone5 | 3.3 | 3.7 |
| 112349 Normal Lung-F | 0.0 | 0.0 | 112673 OA Synovium5 | 1.7 | 1.7 |
| 112357 Normal Lung-F | 0.3 | 0.0 | 112674 OA Synovial Fluid cells5 | 2.6 | 1.0 |
| 112354 Normal Lung-M | 0.3 | 0.0 | 117100 OA Cartilage Rep 14 | 0.0 | 0.0 |
| 112374 Crohns-F | 0.0 | 0.9 | 112756 OA Bone9 | 0.4 | 0.0 |
| 112389 Match Control Crohns- | 1.1 | 0.0 | 112757 OA Synovium9 | 37.4 | 25.0 |
| F | | | | | |
| 112375 Crohns-F | 0.0 | 0.0 | 112758 OA Synovial Fluid Cells9 | 0.6 | 1.2 |
| 112732 Match Control Crohns- | 0.8 | 0.7 | 1117125 RA Cartilage Rep2 | 1.1 | 1.4 |
| F | | | | | |
| 112725 Crohns-M | 0.0 | 0.6 | 113492 Bone2 RA | 1.2 | 1.4 |
| 112387 Match Control Crohns- | 1.1 | 1.1 | 113493 Synovium2 RA | 0.8 | 1.0 |
| M | | | | | |
| 112378 Crohns-M | 0.0 | 0.0 | 1113494 Syn Fluid Cells RA | 0.8 | 1.0 |
| 1112390 Match Control Crohns- | 0.8 | 1.2 | 1113499 Cartilage4 RA | 1.9 | 2.6 |
| M | | | | | |
| 112726 Crohns-M | 0.7 | 1.6 | 113500 Bone4 RA | 2.2 | 1.8 |
| 112731 Match Control Crohns- | 0.3 | 1.7 | 113501 Synovium4 RA | 0.7 | 1.0 |
| M | | | | | |
| 112380 UlcerCol-F | 0.5 | 1.7 | 113502 Syn Fluid Cells4 RA | 0.6 | 1.3 |
| 112734 Match Control Ulcer | 1.8 | 2.2 | 113495 Cartilage3 RA | 0.6 | 1.4 |
| Col-F | | | | | |
| 112384 UlcerCol-F | 1.9 | 1.4 | 113496 Bone3 RA | 0.7 | 1.3 |
| 112737 Match Control Ulcer | 0.6 | 0.0 | 113497 Synovium3 RA | 0.8 | 0.9 |
| Col-F | | | | | |
| 112386 Ulcer Col-F | 1.4 | 1.1 | 113498 Syn Fluid Cells3 RA | 2.7 | 2.5 |
| 112738 Match Control Ulcer | 0.3 | 0.0 | 117106 Normal Cartilage Rep20 | 0.0 | 0.0 |
| Col-F | | | | | |
| 112381 Ulcer Col-M | 0.0 | 0.0 | 113663 Bone3 Normal | 0.0 | 0.0 |
| 112735 Match Control Ulcer | 1.2 | 1.5 | 113664 Synovium3 Normal | 0.0 | 0.0 |
| Col-M | | | | | |
| 112382 Ulcer Col-M | 0.6 | 1.0 | 113665 Syn Fluid Cells3 Normal | 0.0 | 0.0 |
| 112394 Match Control Ulcer | 0.8 | 0.0 | 117107 Normal Cartilage Rep22 | 0.4 | 1.4 |
| Col-M | | | | | |
| 112383 Ulcer Col-M | 0.0 | 0.0 | 113667 Bone4 Normal | 0.0 | 0.0 |
| 112736 Match Control Ulcer | 0.3 | 0.7 | 113668 Synovium4 Normal | 0.5 | 0.9 |
| Col-M | | | | | |
| 112423 Psoriasis-F | 0.3 | 0.0 | 113669 Syn Fluid Cells4 Normal | 0.5 | 1.8 |

TABLE AYD
General_screening_panel v1.5
Column A - Rel. Exp.(%) Ag5035, Run 228967202
Column B - Rel. Exp.(%) Ag5035, Run 244373096
Column C - Rel. Exp.(%) Ag6142, Run 258495052
Column D - Rel. Exp.(%) Ag6142, Run 258496099

| Tissue Name | A | B | C | D |
|---|---|---|---|---|
| Adipose | 1.6 | 2.2 | 1.4 | 0.7 |
| Melanoma* Hs688(A).T | 0.0 | 0.0 | 0.0 | 0.0 |
| Melanoma* Hs688(B).T | 0.0 | 0.0 | 0.0 | 0.0 |
| Melanoma* M14 | 0.0 | 0.0 | 0.0 | 0.0 |
| Melanoma* LOXIMVI | 0.0 | 0.0 | 0.0 | 0.0 |
| Melanoma* SK-MEL-5 | 0.0 | 0.0 | 0.0 | 0.0 |
| Squamous cell carcinoma SCC-4 | 0.0 | 0.0 | 0.0 | 0.0 |
| Testis Pool | 0.0 | 0.1 | 0.6 | 0.5 |
| Prostate ca.* (bone met) PC-3 | 0.0 | 0.0 | 0.0 | 0.0 |
| Prostate Pool | 1.4 | 2.0 | 2.3 | 1.9 |
| Placenta | 0.3 | 0.1 | 0.3 | 0.0 |
| Uterus Pool | 2.1 | 1.6 | 1.1 | 0.8 |
| Ovarian ca. OVCAR-3 | 0.0 | 0.0 | 0.0 | 0.0 |
| Ovarian ca. SK-OV-3 | 0.0 | 0.0 | 0.0 | 0.8 |
| Ovarian ca. OVCAR-4 | 0.3 | 0.0 | 0.5 | 0.0 |
| Ovarian ca. OVCAR-5 | 0.0 | 0.0 | 0.2 | 0.2 |
| Ovarian ca. IGROV-1 | 0.0 | 0.0 | 0.2 | 0.0 |
| Ovarian ca. OVCAR-8 | 0.0 | 0.0 | 0.0 | 0.0 |
| Ovary | 0.3 | 0.3 | 0.1 | 0.0 |
| Breast ca. MCF-7 | 0.0 | 0.0 | 0.0 | 0.0 |
| Breast ca. MDA-MB-231 | 0.0 | 0.0 | 0.4 | 0.0 |
| Breast ca. BT 549 | 0.0 | 0.0 | 0.0 | 0.0 |
| Breast ca. T47D | 0.0 | 0.0 | 0.0 | 0.0 |
| Breast ca. MDA-N | 0.0 | 0.0 | 0.4 | 0.0 |
| Breast Pool | 2.6 | 3.5 | 2.5 | 3.4 |
| Trachea | 0.8 | 1.1 | 2.6 | 2.2 |
| Lung | 0.0 | 0.0 | 0.0 | 0.0 |
| Fetal Lung | 14.3 | 14.5 | 21.3 | 17.7 |
| Lung ca. NCI-N417 | 0.0 | 0.0 | 0.0 | 0.0 |
| Lung ca. LX-1 | 0.0 | 0.0 | 0.0 | 0.0 |
| Lung ca. NCI-H146 | 0.0 | 0.0 | 0.2 | 0.0 |
| Lung ca. SHP-77 | 9.3 | 12.7 | 19.9 | 11.7 |
| Lung ca. A549 | 0.0 | 0.0 | 0.0 | 0.0 |
| Lung ca. NCI-H526 | 0.2 | 0.2 | 0.0 | 0.0 |
| Lung ca. NCI-H23 | 0.0 | 0.0 | 0.2 | 0.0 |
| Lung ca. NCI-H460 | 0.0 | 0.0 | 5.4 | 2.5 |
| Lung Ca. HOP-62 | 0.0 | 0.0 | 0.0 | 0.0 |
| Lung Ca. NCI-H522 | 0.0 | 0.0 | 0.2 | 0.0 |
| Liver | 0.0 | 0.0 | 0.0 | 0.0 |
| Fetal Liver | 2.1 | 2.0 | 1.1 | 1.6 |
| Liver ca. HepG2 | 0.0 | 2.0 | 0.2 | 0.0 |
| Kidney Pool | 1.8 | 0.0 | 1.7 | 2.6 |
| Fetal Kidney | 0.8 | 0.7 | 1.4 | 0.0 |
| Renal ca. 786-0 | 0.0 | 0.0 | 0.0 | 0.0 |
| Renal ca. A498 | 0.0 | 0.0 | 0.0 | 0.0 |
| Renal ca. ACHN | 0.0 | 0.0 | 0.0 | 0.0 |
| Renal ca. UO-31 | 0.0 | 0.0 | 0.0 | 0.0 |
| Renal ca. TK-10 | 0.0 | 0.0 | 0.2 | 0.2 |
| Bladder | 0.6 | 0.3 | 2.0 | 1.3 |
| Gastric ca. (liver met.) NCI-N87 | 0.0 | 0.0 | 0.2 | 0.0 |
| Gastric ca. KATO III | 0.0 | 0.0 | 0.5 | 0.0 |
| Colon ca. SW-948 | 0.0 | 0.0 | 0.2 | 0.0 |
| Colon ca. SW480 | 0.0 | 0.0 | 0.1 | 0.0 |
| Colon ca.* (SW480 met) SW620 | 0.0 | 0.0 | 0.0 | 0.0 |
| Colon ca. HT29 | 0.0 | 0.0 | 0.0 | 0.0 |
| Colon ca. HCT-116 | 0.0 | 0.0 | 0.0 | 0.0 |
| Colon ca. CaCo-2 | 0.2 | 0.2 | 0.0 | 0.0 |
| Colon cancer tissue | 0.7 | 0.1 | 0.9 | 0.0 |
| Colon ca. SW1116 | 0.0 | 0.0 | 0.0 | 0.0 |
| Colon ca. Colo-205 | 0.0 | 0.0 | 0.0 | 0.0 |
| Colon ca. SW-48 | 0.0 | 0.0 | 0.0 | 0.0 |
| Colon Pool | 3.5 | 3.1 | 3.2 | 1.9 |
| Small Intestine Pool | 1.1 | 1.3 | 1.8 | 0.7 |
| Stomach Pool | 0.2 | 1.4 | 0.4 | 0.3 |
| Bone Marrow Pool | 2.3 | 1.8 | 1.4 | 2.4 |
| Fetal Heart | 0.3 | 0.6 | 0.8 | 0.6 |
| Heart Pool | 0.8 | 0.0 | 2.2 | 0.7 |
| Lymph Node Pool | 2.6 | 2.0 | 3.0 | 4.8 |
| Fetal Skeletal Muscle | 17.9 | 22.2 | 20.9 | 27.0 |
| Skeletal Muscle Pool | 100.0 | 83.5 | 96.6 | 79.0 |
| Spleen Pool | 0.6 | 0.0 | 0.2 | 0.5 |
| Thymus Pool | 0.6 | 0.4 | 1.5 | 1.1 |
| CNS cancer (glio/astro) U87-MG | 0.0 | 0.0 | 1.5 | 1.2 |
| CNS cancer (glio/astro) U-118-MG | 0.2 | 0.0 | 0.0 | 0.0 |
| CNS cancer (neuro; met) SK-N-AS | 0.0 | 0.0 | 0.0 | 0.0 |
| CNS cancer (astro) SF-539 | 0.0 | 0.0 | 0.0 | 0.0 |
| CNS cancer (astro) SNB-75 | 0.0 | 0.2 | 0.9 | 1.0 |
| CNS cancer (glio) SNB-19 | 0.0 | 0.0 | 0.0 | 0.0 |
| CNS cancer (glio) SF-295 | 0.0 | 0.0 | 0.0 | 0.0 |
| Brain (Amygdala) Pool | 32.8 | 31.0 | 36.3 | 26.2 |
| Brain (cerebellum) | 69.7 | 76.3 | 61.1 | 49.3 |
| Brain (fetal) | 90.1 | 100.0 | 100.0 | 100.0 |
| Brain (Hippocampus) Pool | 27.9 | 31.0 | 32.1 | 29.3 |
| Cerebral Cortex Pool | 36.3 | 48.3 | 39.5 | 42.3 |
| Brain (Substantia nigra) Pool | 31.0 | 32.1 | 28.3 | 24.8 |
| Brain (Thalamus) Pool | 50.0 | 50.3 | 54.7 | 51.1 |
| Brain (whole) | 46.0 | 38.2 | 50.7 | 46.7 |
| Spinal Cord Pool | 17.6 | 18.4 | 25.2 | 17.7 |
| Adrenal Gland | 2.5 | 2.5 | 3.1 | 2.1 |
| Pituitary gland Pool | 1.3 | 1.2 | 1.4 | 3.2 |
| Salivary Gland | 0.0 | 0.2 | 0.2 | 0.0 |
| Thyroid (female) | 0.0 | 0.0 | 0.3 | 0.2 |
| Pancreatic ca. CAPAN2 | 0.0 | 0.0 | 0.0 | 0.0 |
| Pancreas Pool | 1.6 | 7.9 | 1.3 | 1.6 |

TABLE AYE
Panel 4.1D
Column A - Rel. Exp.(%) Ag5035, Run 223740981

| Tissue Name | A | Tissue Name | A |
|---|---|---|---|
| Secondary Th1 act | 0.0 | HUVEC IL-1beta | 2.0 |
| Secondary Th2 act | 0.0 | HUVEC IFN gamma | 0.0 |
| Secondary Tr1 act | 0.0 | HUVEC TNF alpha + IFN gamma | 1.9 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 100.0 |
| | | 1beta | |
| Primary Th2 act | 0.0 | Microvascular Dermal EC none | 0.0 |
| Primary Tr1 act | 2.0 | Microsvasular Dermal EC TNFalpha + IL- | 55.5 |
| | | 1beta | |
| Primary Th1 rest | 0.0 | Bronchial epithelium TNFalpha +IL1beta | 2.2 |
| Primary Th2 rest | 0.0 | Small airway epithelium none | 4.2 |
| Primary Tr1 rest | 0.0 | Small airway epithelium TNFalpha + IL- | 3.7 |
| | | 1beta | |
| CD45RA CD4 lymphocyte act | 0.0 | Coronery artery SMC rest | 2.3 |
| CD45RO CD4 lymphocyte act | 0.0 | Coronery artery SMC TNFalpha + IL-1beta | 0.0 |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 10.6 |
| CD4 lymphocyte none | 0.0 | KU-812 (Basophil) PMA/ionomycin | 13.6 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 0.0 |
| LAK cells IL-2 + IL-12 | 8.4 | NCI-H292 none | 0.0 |
| LAK cells IL-2 + IFN gamma | 2.9 | NCI-H292 IL-4 | 0.0 |
| LAK cells IL-2 + IL-18 | 3.8 | NCI-H292 IL-9 | 0.0 |
| LAK cells PMA/ionomycin | 2.3 | NCI-H292 IL-13 | 2.5 |
| NK Cells IL-2 rest | 0.0 | NCI-H292 IFN gamma | 0.0 |
| Two Way MLR 3 day | 2.1 | HPAEC none | 0.0 |
| Two Way MLR 5 day | 2.3 | HPAEC TNF alpha + IL-1 beta | 8.8 |
| Two Way MLR 7 day | 1.8 | Lung fibroblast none | 0.0 |
| PBMC rest | 6.7 | Lung fibroblast TNF alpha + IL-1 beta | 0.0 |
| PBMC PWM | 0.0 | Lung fibroblast IL-4 | 0.0 |
| PBMC PHA-L | 6.3 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 9.2 | Dermal fibroblast CCD1070 rest | 0.0 |
| B lymphocytes CD40L and IL-4 | 1.9 | Dermal fibroblast CCD1070 TNF alpha | 0.0 |
| EOL-1 dbcAMP | 0.0 | Dermal fibroblast CCD1070 IL-1 beta | 0.0 |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 2.4 | Dermal Fibroblasts rest | 2.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 31.6 | Colon | 5.4 |
| Macrophages rest | 1.7 | Lung | 8.1 |
| Macrophages LPS | 6.5 | Thymus | 5.1 |
| HUVEC none | 0.0 | Kidney | 0.0 |
| HUVEC starved | 0.0 | | |

TABLE AYF
Panel 5 Islet
Column A - Rel. Exp.(%) Ag5035, Run 244908253

| Tissue Name | A | Tissue Name | A |
|---|---|---|---|
| 97457 Patient-02go adipose | 1.4 | 94709 Donor 2 AM - A adipose | 0.0 |
| 97476 Patient-07sk skeletal muscle | 3.2 | 94710 Donor 2 AM - B adipose | 0.0 |
| 97477 Patient-07ut uterus | 0.0 | 94711 Donor 2 AM - C adipose | 0.0 |
| 97478 Patient-07pl placenta | 1.1 | 94712 Donor 2 AD - A adipose | 0.0 |
| 99167 Bayer Patient 1 | 24.8 | 94713 Donor 2 AD - B adipose | 0.0 |
| 97482 Patient-08ut uterus | 0.0 | 94714 Donor 2 AD - C adipose | 0.0 |
| 97483 Patient-08pl placenta | 0.0 | 94742 Donor 3 U - A Mesenchymal | 0.0 |
| | | Stem Cells | |
| 97486 Patient-09sk skeletal muscle | 11.1 | 94743 Donor 3 U - B Mesenchymal | 0.0 |
| | | Stem Cells | |
| 97487 Patient-09ut uterus | 1.7 | 94730 Donor 3 AM - A adipose | 0.0 |
| 97488 Patient-09pl placenta | 1.1 | 94731 Donor 3 AM - B adipose | 0.0 |
| 97492 Patient-10ut uterus | 0.0 | 94732 Donor 3 AM - C adipose | 0.0 |
| 97493 Patient-10pl placenta | 1.1 | 94733 Donor 3 AD - A adipose | 0.0 |
| 97495 Patient-11go adipose | 0.0 | 94734 Donor 3 AD - B adipose | 0.0 |
| 97496 Patient-11sk skeletal muscle | 33.9 | 94735 Donor 3 AD - C adipose | 0.0 |
| 97497 Patient-11ut uterus | 0.0 | 77138 Liver HepG2untreated | 0.0 |
| 97498 Patient-11pl placenta | 0.0 | 73556 Heart Cardiac stromal cells | 0.0 |
| | | (primary) | |
| 97500 Patient-12go adipose | 0.0 | 81735 Small Intestine | 0.0 |
| 97501 Patient-12sk skeletal muscle | 100.0 | 72409 Kidney Proximal Convoluted | 0.0 |
| | | Tubule | |
| 97502 Patient-12ut uterus | 0.0 | 82685 Small intestine Duodenum | 1.1 |
| 97503 Patient-12pl placenta | 0.0 | 90650 Adrenal Adrenocortical | 0.0 |
| | | adenoma | |
| 94721 Donor 2 U - A Mesenchymal | 0.0 | 72410 Kidney HRCE | 1.6 |
| Stem Cells | | | |
| 94722 Donor 2 U - B Mesenchymal | 0.0 | 72411 Kidney HRE | 0.0 |
| Stem Cells | | | |
| 94723 Donor 2 U - C Mesenchymal | 0.0 | 73139 Uterus Uterine smooth muscle | 0.0 |
| Stem Cells | | cells | |

AI_comprehensive panel_v1.0 Summary: Ag5035/Ag6142 The highest expression of this gene was detected in an OA bone sample. Expression of the The expression of this gene was highly associated with synovium and bone samples from patients with osteoarthritis when compared to expression in the control samples. Therapeutic modulation of this gene, expressed protein and/or use of antibodies or small molecule drugs targeting the gene or gene product are useful in the treatment of osteoarthritis.

General_screening_panel_v1.5 Summary: Ag5035/Ag6142 This gene showed highly brain preferential expression (CTs=30-31). Inhibition of calcium uptake has been shown to decrease neuronal death in response to cerebral ischemia. Therapeutic modulation of this gene, expressed protein and/or use of antibodies or small molecule drugs targeting the gene or gene product are useful in the treatment of stroke by decreasing the total infarct volume.

Moderate levels of expression were seen in fetal and adult skeletal muscle (CTs=30-31). This gene encodes a putative sodium/calcium exchanger. Altered levels of intracellular calcium have been implicated in many diseases, including type 2 diabetes. Based on its expression profile and homology to a calcium transport protein, therapeutic modulation of this gene, expressed protein and/or use of antibodies or small molecule drugs targeting the gene or gene product are useful in the treatment of type 2 diabetes.

Panel 4.1D Summary: Ag5035 Expression of this gene was restricted to TNF-alpha and IL-1 beta treated lung and dermal microvasculature (CTs=33-34). Endothelial cells are known to play important roles in inflammatory responses by altering the expression of surface proteins that are involved in activation and recruitment of effector inflammatory cells. The expression of this gene in dermal microvascular endothelial cells indicated that this protein product may be involved in inflammatory responses to skin disorders, including psoriasis. Expression in lung microvascular endothelial cells indicated that the protein encoded by this transcript may also be involved in lung disorders including asthma, allergies, chronic obstructive pulmonary disease, and emphysema. Therapeutic modulation of this gene, expressed protein and/or use of antibodies or small molecule drugs targeting the gene or gene product are useful in the treatment of psoriasis, asthma, allergies, chronic obstructive pulmonary disease, and emphysema.

Panel 5 Islet Summary: Ag5035 Expression of this gene was highest and most prominent in sampels derived from skeletal muscle (CTs=29-33). Please see Panel 1.5 for discussion of this gene in metabolic disease.

AZ. CG56262-01: Ca-Binding Transporter.

Expression of gene CG56262-01 was assessed using the primer-probe sets Ag2896 and Ag2920, described in Tables AZA and AZB. Results of the RTQ-PCR runs are shown in Tables AZC, AZD, AZE, AZF and AZG.

TABLE AZA
Probe Name Ag2896

| | | | Start | SEQ ID |
|---|---|---|---|---|
| Primers | Sequences | Length | Position | No |
| Forward | 5′-gtcagcttctcttgctttgaga-3′ | 22 | 900 | 1344 |
| Probe | TET-5′-cactgtcaggcactcgccaatgt-3′- | 23 | 932 | 1345 |
| | TAMRA | | | |
| Reverse | 5′-ctgtatttctggaagcattcca-3′ | 22 | 964 | 1346 |

TABLE AZB
Probe Name Ag2920

| | | | Start | SEQ ID |
|---|---|---|---|---|
| Primers | Sequences | Length | Position | No |
| Forward | 5′-ttgatgtctctgagatccaaca-3′ | 22 | 1134 | 1347 |
| Probe | TET-5′-agtttccgagctctgggcatttccat-3′- | 26 | 1107 | 1348 |
| | TAMRA | | | |
| Reverse | 5′-catgctgtgcaaaattttctc-3′ | 21 | 1070 | 1349 |

TABLE AZC
CNS_neurodegeneration_v1.0
Column A - Rel. Exp.(%) Ag2896, Run 209734744
Column B - Rel. Exp.(%) Ag2920, Run 209779301

| Tissue Name | A | B | Tissue Name | A | B |
|---|---|---|---|---|---|
| AD 1 Hippo | 46.2 | 55.1 | AH3 4624 | 45.4 | 64.6 |
| AD 2 Hippo | 65.1 | 62.0 | AH3 4640 | 61.6 | 56.6 |
| AD 3 Hippo | 49.0 | 64.2 | AD 1 Occipital Ctx | 24.3 | 30.1 |
| AD 4 Hippo | 34.2 | 37.6 | AD 2 Occipital Ctx (Missing) | 0.3 | 0.3 |
| AD 5 Hippo | 52.5 | 46.3 | AD 3 Occipital Ctx | 32.1 | 35.4 |
| AD 6 Hippo | 59.9 | 67.8 | AD 4 Occipital Ctx | 46.0 | 53.2 |
| Control 2 Hippo | 76.3 | 87.1 | AD 5 Occipital Ctx | 60.3 | 29.5 |
| Control 4 Hippo | 38.7 | 48.3 | AD 5 Occipital Ctx | 28.7 | 55.9 |
| Control (Path) 3 Hippo | 38.7 | 47.0 | Control 1 Occipital Ctx | 32.5 | 40.9 |
| AD 1 Temporal Ctx | 38.2 | 41.5 | Control 2 Occipital Ctx | 60.3 | 66.4 |
| AD 2 Temporal Ctx | 62.9 | 65.1 | Control 3 Occipital Ctx | 29.9 | 42.6 |
| AD 3 Temporal Ctx | 39.0 | 49.7 | Control 4 Occipital Ctx | 38.4 | 43.8 |
| AD 4 Temporal Ctx | 55.5 | 52.5 | Control (Path) 1 Occipital Ctx | 67.8 | 85.3 |
| AD 5 Inf Temporal Ctx | 59.9 | 61.1 | Control (Path) 2 Occipital Ctx | 31.6 | 32.3 |
| AD 5 Sup Temporal Ctx | 49.7 | 45.7 | Control (Path) 3 Occipital Ctx | 29.9 | 40.1 |
| AD 6 Inf Temporal Ctx | 48.6 | 48.0 | Control (Path) 4 Occipital Ctx | 30.4 | 27.2 |
| AD 6 Sup Temporal Ctx | 45.1 | 52.5 | Control 1 Parietal Ctx | 46.7 | 51.8 |
| Control 1 Temporal Ctx | 55.5 | 62.9 | Control 2 Parietal Ctx | 48.3 | 46.7 |
| Control 2 Temporal Ctx | 81.2 | 99.3 | Control 3 Parietal Ctx | 6.5 | 40.9 |
| Control 3 Temporal Ctx | 55.1 | 54.0 | Control (Path) 1 Parietal Ctx | 100.0 | 100.0 |
| Control 3 Temporal Ctx | 52.9 | 47.3 | Control (Path) 2 Parietal Ctx | 43.2 | 42.0 |
| AH3 3975 | 94.6 | 99.3 | Control (Path) 3 Parietal Ctx | 33.0 | 47.3 |
| AH3 3954 | 64.2 | 72.2 | Control (Path) 4 Parietal Ctx | 57.4 | 62.0 |

TABLE AZD
Panel 1.3D
Column A - Rel. Exp.(%) Ag2896, Run 167660338
Column B - Rel. Exp.(%) Ag2920, Run 167646813

| Tissue Name | A | B | Tissue Name | A | B |
|---|---|---|---|---|---|
| Liver adenocarcinoma | 36.6 | 40.1 | Kidney (fetal) | 23.2 | 21.6 |
| Pancreas | 4.2 | 7.4 | Renal ca. 786-0 | 15.5 | 19.6 |
| Pancreatic ca. CAPAN 2 | 10.1 | 9.3 | Renal ca. A498 | 9.5 | 9.4 |
| Adrenal gland | 3.3 | 2.8 | Renal ca. RXF 393 | 17.3 | 16.6 |
| Thyroid | 11.8 | 18.9 | Renal ca. ACHN | 10.5 | 14.5 |
| Salivary gland | 6.7 | 6.6 | Renal ca. UO-31 | 7.7 | 9.9 |
| Pituitary gland | 2.2 | 2.7 | Renal ca. TK-10 | 12.4 | 14.7 |
| Brain (fetal) | 27.0 | 27.7 | Liver | 4.3 | 3.5 |
| Brain (whole) | 81.2 | 74.2 | Liver (fetal) | 1.8 | 2.6 |
| Brain (amygdala) | 40.1 | 40.3 | Liver ca. (hepatoblast) HepG2 | 4.7 | 4.9 |
| Brain (cerebellum) | 30.8 | 33.0 | Lung | 5.4 | 3.2 |
| Brain (hippocampus) | 44.8 | 42.0 | Lung (fetal) | 4.8 | 4.7 |
| Brain (substantia nigra) | 23.0 | 21.5 | Lung ca. (small cell) LX-1 | 6.7 | 6.2 |
| Brain (thalamus) | 25.5 | 31.6 | Lung ca. (small cell) NCI-H69 | 0.0 | 0.1 |
| Cerebral Cortex | 100.0 | 100.0 | Lung ca. (s.cell var.) SHP-77 | 26.1 | 31.9 |
| Spinal cord | 12.6 | 12.9 | Lung ca. (large cell) NCI-H460 | 1.2 | 1.4 |
| glio/astro U87-MG | 2.2 | 2.4 | Lung ca. (non-sm. cell) A549 | 10.4 | 8.9 |
| glio/astro U-118-MG | 9.9 | 8.1 | Lung ca. (non-s.cell) NCI-H23 | 11.0 | 12.7 |
| astrocytoma SW1783 | 8.8 | 10.1 | Lung ca. (non-s.cell) HOP-62 | 4.9 | 4.7 |
| neuro*; met SK-N-AS | 4.2 | 3.3 | Lung ca. (non-s.cl) NCI-H522 | 11.4 | 11.4 |
| astrocytoma SF-539 | 5.8 | 5.4 | Lung ca. (squam.) SW 900 | 7.9 | 8.6 |
| astrocytoma SNB-75 | 10.2 | 10.5 | Lung ca. (squam.) NCI-H596 | 0.3 | 0.4 |
| glioma SNB-19 | 10.1 | 11.0 | Mammary gland | 8.3 | 8.5 |
| glioma U251 | 14.1 | 15.8 | Breast ca.* (pl.ef) MCF-7 | 7.5 | 8.1 |
| glioma SF-295 | 6.0 | 5.9 | Breast ca.* (pl.ef) MDA-MB- | 6.6 | 7.1 |
| | | | 231 | | |
| Heart (Fetal) | 38.7 | 40.1 | Breast ca.* (pl.ef) T47D | 16.2 | 17.0 |
| Heart | 10.7 | 9.9 | Breast ca. BT-549 | 5.8 | 5.1 |
| Skeletal muscle (Fetal) | 16.0 | 11.8 | Breast ca. MDA-N | 19.5 | 22.5 |
| Skeletal muscle | 31.2 | 28.7 | Ovary | 10.4 | 10.3 |
| Bone marrow | 0.4 | 0.6 | Ovarian ca. OVCAR-3 | 11.5 | 9.2 |
| Thymus | 2.5 | 2.5 | Ovarian ca. OVCAR-4 | 35.6 | 32.5 |
| Spleen | 1.1 | 1.4 | Ovarian ca. OVCAR-5 | 31.0 | 34.2 |
| Lymph node | 2.6 | 1.7 | Ovarian ca. OVCAR-8 | 5.4 | 5.5 |
| Colorectal | 14.0 | 12.3 | Ovarian ca. IGROV-1 | 10.3 | 10.2 |
| Stomach | 4.4 | 4.0 | Ovarian ca. (ascites) SK-OV-3 | 13.8 | 18.0 |
| Small intestine | 4.4 | 4.9 | Uterus | 6.3 | 8.4 |
| Colon ca. SW480 | 5.1 | 5.3 | Placenta | 0.0 | 0.0 |
| Colon ca.* SW620 (SW480 met) | 14.9 | 18.7 | Prostate | 3.2 | 3.8 |
| Colon ca. HT29 | 6.8 | 7.2 | Prostate ca.* (bone met) PC-3 | 16.5 | 18.3 |
| Colon ca. HCT-116 | 10.7 | 10.4 | Testis | 1.4 | 1.3 |
| Colon ca. CaCo-2 | 17.0 | 21.9 | Melanoma Hs688(A).T | 2.8 | 2.6 |
| CC Well to Mod Duff | 2.4 | 2.4 | Melanoma* (met) Hs688(B).T | 2.6 | 3.8 |
| (ODO3866) | | | | | |
| Colon ca. HCC-2998 | 9.3 | 7.6 | Melanoma UACC-62 | 10.9 | 11.2 |
| Gastric ca. (liver met) NCI-N87 | 5.7 | 5.5 | Melanoma M14 | 8.6 | 5.8 |
| Bladder | 5.1 | 5.1 | Melanoma LOX IMVI | 12.2 | 10.8 |
| Trachea | 1.9 | 2.8 | Melanoma* (met) SK-MEL-5 | 24.0 | 25.2 |
| Kidney | 12.4 | 17.0 | Adipose | 6.1 | 6.4 |

TABLE AZE
Panel 4D
Column A - Rel. Exp.(%) Ag2896, Run 164401737
Column B - Rel. Exp.(%) Ag2920, Run 164403312

| Tissue Name | A | B | Tissue Name | A | B |
|---|---|---|---|---|---|
| Secondary Th1 act | 5.6 | 7.3 | HUVEC IL-1beta | 4.9 | 4.3 |
| Secondary Th2 act | 6.8 | 6.0 | HUVEC IFN gamma | 19.6 | 19.8 |
| Secondary Tr1 act | 7.4 | 7.7 | HUVEC TNF alpha + IFN gamma | 7.8 | 8.9 |
| Secondary Th1 rest | 6.4 | 6.7 | HUVEC TNF alpha + IL4 | 6.1 | 7.9 |
| Secondary Th2 rest | 7.6 | 7.1 | HUVEC IL-11 | 10.4 | 11.8 |
| Secondary Tr1 rest | 12.2 | 9.7 | Lung Microvascular EC none | 7.5 | 9.8 |
| Primary Th1 act | 12.7 | 14.5 | Lung Microvascular EC TNFalpha + | 5.5 | 6.1 |
| | | | IL-1beta | | |
| Primary Th2 act | 16.0 | 15.1 | Microvascular Dermal EC none | 13.7 | 12.6 |
| Primary Tr1 act | 26.4 | 22.1 | Microsvasular Dermal EC TNFalpha | 5.7 | 6.6 |
| | | | + IL-1beta | | |
| Primary Th1 rest | 31.6 | 33.4 | Bronchial epithelium TNFalpha + | 15.9 | 11.9 |
| | | | IL1beta | | |
| Primary Th2 rest | 19.3 | 18.7 | Small airway epithelium none | 4.7 | 5.3 |
| Primary Tr1 rest | 14.7 | 16.5 | Small airway epithelium TNFalpha | 35.6 | 37.1 |
| | | | + IL-1beta | | |
| CD45RA CD4 lymphocyte | 6.2 | 5.1 | Coronery artery SMC rest | 7.1 | 6.7 |
| act | | | | | |
| CD45RO CD4 lymphocyte | 11.2 | 12.3 | Coronery artery SMC TNFalpha + | 4.6 | 5.9 |
| act | | | IL-1beta | | |
| CD8 lymphocyte act | 11.6 | 10.8 | Astrocytes rest | 27.0 | 23.8 |
| Secondary CD8 lymphocyte | 8.2 | 9.9 | Astrocytes TNFalpha + IL-1beta | 30.8 | 28.1 |
| rest | | | | | |
| Secondary CD8 lymphocyte | 3.0 | 3.3 | KU-812 (Basophil) rest | 8.2 | 6.3 |
| act | | | | | |
| CD4 lymphocyte none | 2.8 | 3.4 | KU-812 (Basophil) PMA/ionomycin | 22.5 | 19.9 |
| 2ry Th1/Th2/Tr1 anti-CD95 | 7.3 | 7.4 | CCD1106 (Keratinocytes) none | 11.11 | 1.7 |
| CH11 | | | | | |
| LAK cells rest | 7.1 | 6.7 | CCD1106 (Keratinocytes) TNFalpha | 6.8 | 6.3 |
| | | | + IL-1beta | | |
| LAK cells IL-2 | 14.7 | 17.0 | Liver cirrhosis | 3.0 | 3.0 |
| LAK cells IL-2 + IL-12 | 6.9 | 7.0 | Lupus kidney | 6.5 | 6.2 |
| LAK cells IL-2 + IFN | 12.8 | 11.0 | NCI-H292 none | 63.3 | 72.7 |
| gamma | | | | | |
| LAK cells IL-2 + IL-18 | 6.7 | 9.0 | NCI-H292 IL-4 | 57.4 | 69.7 |
| LAK cells PMA/ionomycin | 0.9 | 0.6 | NCI-H292 IL-9 | 57.0 | 65.5 |
| NK Cells IL-2 rest | 7.2 | 6.5 | NCI-H292 IL-13 | 30.8 | 35.6 |
| Two Way MLR 3 day | 6.3 | 6.8 | NCI-H292 IFN gamma | 29.3 | 34.4 |
| Two Way MLR 5 day | 3.5 | 3.0 | HPAEC none | 10.8 | 11.7 |
| Two Way MLR 7 day | 4.2 | 4.5 | HPAEC TNF alpha + IL-1 beta | 6.9 | 6.4 |
| PBMC rest | 2.0 | 1.6 | Lung fibroblast none | 16.8 | 17.4 |
| PBMC PWM | 27.9 | 26.2 | Lung fibroblast TNF alpha + IL-1 | 7.4 | 8.0 |
| | | | beta | | |
| PBMC PHA-L | 27.5 | 26.8 | Lung fibroblast IL-4 | 30.6 | 34.6 |
| Ramos (B cell) none | 16.8 | 16.0 | Lung fibroblast IL-9 | 24.8 | 24.1 |
| Ramos (B cell) ionomycm | 100.0 | 100.0 | Lung fibroblast IL-13 | 19.6 | 21.8 |
| B lymphocytes PWM | 36.6 | 22.8 | Lung fibroblast IFN gamma | 31.4 | 37.6 |
| B lymphocytes CD40L and | 13.5 | 14.9 | Dermal fibroblast CCD1070 rest | 10.7 | 12.0 |
| IL-4 | | | | | |
| EOL-1 dbcAMP | 14.5 | 15.5 | Dermal fibroblast CCD1070 | 20.6 | 21.3 |
| | | | alpha | | |
| EOL-1 dbcAMP | 7.1 | 6.3 | Dermal fibroblast CCD1070 IL-1 | 6.5 | 5.9 |
| PMA/ionomycin | | | beta | | |
| Dendritic cells none | 0.8 | 1.5 | Dermal fibroblast IFN gamma | 10.1 | 11.3 |
| Dendritic cells LPS | 0.1 | 0.2 | Dermal fibroblast IL-4 | 23.0 | 23.2 |
| Dendritic cells anti-CD40 | 0.9 | 0.7 | IBD Colitis 2 | 2.0 | 2.3 |
| Monocytes rest | 0.1 | 0.0 | IBD Crohn's | 3.4 | 4.8 |
| Monocytes LPS | 0.2 | 0.0 | Colon | 41.5 | 50.7 |
| Macrophages rest | 4.0 | 3.4 | Lung | 15.8 | 17.2 |
| Macrophages LPS | 0.5 | 0.4 | Thymus | 57.8 | 55.5 |
| HUVEC none | 12.2 | 12.8 | Kidney | 5.0 | 8.5 |
| HUVEC starved | 21.6 | 20.4 | | | |

TABLE AZF
Panel 5 Islet
Column A - Rel. Exp.(%) Ag2896, Run 268363565

| Tissue Name | A | Tissue Name | A |
|---|---|---|---|
| 97457 Patient-02go adipose | 19.6 | 94709 Donor 2 AM - A adipose | 8.3 |
| 97476 Patient-07sk skeletal muscle | 6.6 | 94710 Donor 2 AM - B adipose | 8.1 |
| 97477 Patient-07ut uterus | 31.6 | 94711 Donor 2 AM - C adipose | 5.0 |
| 97478 Patient-07pl placenta | 2.6 | 94712 Donor 2 AD - A adipose | 17.6 |
| 99167 Bayer Patient 1 | 32.5 | 94713 Donor 2 AD - B adipose | 29.5 |
| 97482 Patient-08ut uterus | 26.6 | 94714 Donor 2 AD - C adipose | 25.7 |
| 97483 Patient-08pl placenta | 1.2 | 94742 Donor 3 U - A Mesenchymal | 5.7 |
| | | Stem Cells | |
| 97486 Patient-09sk skeletal muscle | 8.3 | 94743 Donor 3 U - B Mesenchymal | 5.1 |
| | | Stem Cells | |
| 97487 Patient-09ut uterus | 47.6 | 94730 Donor 3 AM - A adipose | 8.7 |
| 97488 Patient-09pl placenta | 1.3 | 94731 Donor 3 AM - B adipose | 4.4 |
| 97492 Patient-10ut uterus | 28.3 | 94732 Donor 3 AM - C adipose | 3.4 |
| 97493 Patient-10pl placenta | 3.0 | 94733 Donor 3 AD - A adipose | 9.9 |
| 97495 Patient-11go adipose | 47.6 | 94734 Donor 3 AD - B adipose | 4.4 |
| 97496 Patient-11sk skeletal muscle | 35.1 | 94735 Donor 3 AD - C adipose | 4.6 |
| 97497 Patient-11ut uterus | 100.0 | 77138 Liver HepG2untreated | 54.7 |
| 97498 Patient-11pl placenta | 0.4 | 73556 Heart Cardiac stromal cells | 10.6 |
| | | (primary) | |
| 97500 Patient-12go adipose | 17.3 | 81735 Small Intestine | 60.7 |
| 97501 Patient-12sk skeletal muscle | 37.4 | 72409 Kidney Proximal Convoluted | 24.1 |
| | | Tubule | |
| 97502 Patient-12ut uterus | 76.3 | 82685 Small intestine Duodenum | 18.8 |
| 97503 Patient-12pl placenta | 0.9 | 90650 Adrenal Adrenocortical | 3.8 |
| | | adenoma | |
| 94721 Donor 2 U - A Mesenchymal | 15.2 | 72410 Kidney HRCE | 70.2 |
| Stem Cells | | | |
| 94722 Donor 2 U - B Mesenchymal | 11.9 | 72411 Kidney HRE | 59.5 |
| Stem Cells | | | |
| 94723 Donor 2 U - C Mesenchymal | 9.7 | 73139 Uterus Uterine smooth muscle | 8.7 |
| Stem Cells | | cells | |

TABLE AZG
general oncology screening panel_v_2.4
Column A - Rel. Exp. (%) Ag2896, Run 260442400
Column B - Rel. Exp. (%) Ag2920, Run 260443377

| Tissue Name | A | B | Tissue Name | A | B |
|---|---|---|---|---|---|
| Colon cancer 1 | 26.6 | 28.1 | Bladder NAT 2 | 0.5 | 0.5 |
| CC Margin (ODO3921) | 29.9 | 29.1 | Bladder NAT 3 | 0.2 | 0.1 |
| Colon cancer 2 | 26.6 | 18.0 | Bladder NAT 4 | 13.4 | 11.0 |
| Colon NAT 2 | 25.7 | 20.2 | Prostate adenocarcinoma 1 | 35.8 | 18.4 |
| Colon cancer 3 | 52.5 | 59.0 | Prostate adenocarcinoma 2 | 3.3 | 2.0 |
| Colon NAT 3 | 74.7 | 71.2 | Prostate adenocarcinoma 3 | 19.1 | 16.0 |
| Colon malignant cancer 4 | 54.0 | 53.2 | Prostate adenocarcinoma 4 | 13.2 | 13.1 |
| Colon NAT 4 | 28.7 | 16.0 | Prostate NAT 5 | 2.4 | 2.8 |
| Lung cancer 1 | 11.4 | 9.9 | Prostate adenocarcinoma 6 | 7.9 | 5.3 |
| Lung NAT 1 | 1.6 | 0.5 | Prostate adenocarcinoma 7 | 8.0 | 5.2 |
| Lung cancer 2 | 64.2 | 66.0 | Prostate adenocarcinoma 8 | 1.7 | 1.4 |
| Lung NAT 2 | 1.5 | 0.6 | Prostate adenocarcinoma 9 | 39.8 | 29.1 |
| Squamous cell carcinoma 3 | 25.9 | 25.2 | Prostate NAT 10 | 2.5 | 0.8 |
| Lung NAT 3 | 0.6 | 0.4 | Kidney cancer 1 | 18.9 | 18.4 |
| Metastatic melanoma 1 | 32.8 | 22.5 | Kidney NAT 1 | 15.7 | 12.5 |
| Melanoma 2 | 1.8 | 0.8 | Kidney cancer 2 | 100.0 | 100.0 |
| Melanoma 3 | 2.1 | 1.2 | Kidney NAT 2 | 29.7 | 29.7 |
| Metastatic melanoma 4 | 37.6 | 44.8 | Kidney cancer 3 | 18.0 | 22.8 |
| Metastatic melanoma 5 | 79.6 | 72.7 | Kidney NAT 3 | 15.3 | 14.7 |
| Bladder cancer 1 | 1.0 | 0.3 | Kidney cancer 4 | 31.0 | 28.3 |
| Bladder NAT 1 | 0.0 | 0.0 | Kidney NAT 4 | 19.6 | 21.9 |
| Bladder cancer 2 | 8.3 | 5.7 | | | |

CNS_neurodegeneration_v1.0 Summary: Ag2896/Ag2920 This gene was found to be down-regulated in the temporal cortex of Alzheimer's disease patients. Up-regulation of this gene or its protein product, or treatment with specific agonists for this receptor is of use in reversing the dementia, memory loss and neuronal death associated with this disease.

Panel 1.3D Summary: Ag2896/Ag2920 Highest expression of this gene was detected in the cerebral cortex (CTs=26). High expression of this gene was seen predominantly in all the regions of the central nervous system examined, including amygdala, hippocampus, substantia nigra, thalamus, cerebellum, cerebral cortex, and spinal cord. This gene encodes a Ca binding transporter. Ca++ is critical for synaptic vesicle release (Kovacs I, Neurochem Int 1998 November; 33(5):399-405). Targeting this gene with a small molecule drug, protein therapeutic or antibody is useful for the treatment of diseases resulting from altered/inappropriate synaptic transmission such as epilepsy, schizophrenia, bipolar disorder, depression, and mania.

This gene also had moderate levels of expression adult and fetal heart, skeletal muscle and liver, and adipose. This gene product is homologous to a mitochondrial calcium-dependent transporter. Since intracellular calcium homeostasis is critically important for energy metabolism and signal transduction, modulation of this gene or gene product is useful as a therapeutic for metabolic and endocrine diseases.

Moderate expression was also seen in almost all the cancer cell lines on this panel. This shows that expression of this gene product is required for cell growth and proliferaton in almost all cell types.

Panel 4D Summary: Ag2896/Ag2920 Moderate to low expression of this gene was detected across a wide range of cells on this panel including epithelium, fibroblasts, and endothelial cells. Lower but still significant levels of expression were also seen in monocytes/macrophages, T and B cells, which all play an importan role in both innate and adaptive immunity. Expression of this gene was highest in the B cell lymphoma cell line, and the NCI H292 mucoepidermoid cell line (CTs=26.4-27). Inhibition of the function of the protein encoded by this transcript with a small molecule drug, protein therapeutic, or antibody is useful for the reduction of the symptoms of patients suffering from autoimmune and inflammatory diseases such as asthma, COPD, emphysema, psoriasis, inflammatory bowel disease, lupus erythematosus, or rheumatoid arthritis.

Panel 5 Islet Summary: Ag2896 This gene showed widespread expression in this panel with highest expression seen in uterus from a non-diabetic patient (CT=28.8). Significant expression of this gene was seen in adipose, skeletal muscle, uterus, kidney, small intestine and a liver cancer cell line, which is in agreement with expression seen in panel 1.3D.

general oncology screening panel_v_{—}2.4 Summary: Ag2896/Ag2920 Highest expression of this gene was detected in a kidney cancer sample (CTs=27). Prominent expression of this gene was also seen in melanoma and prostate cancer samples. This gene was overexpressed in lung cancer samples when compared to expression in matched normal adjacent tissue. Expression of this gene or its protein product is useful as a marker of lung cancers. Targeting this gene or its protein product with a small molecule drug, protein therapeutic, or antibody is useful in the treatment of these cancers.

BA. CG56398-01: Na/Glucose Cotransporter.

Expression of gene CG56398-01 was assessed using the primer-probe set Ag2925, described in Table BAA. Results of the RTQ-PCR runs are shown in Tables BAB, BAC, BAD and BAE.

TABLE BAA
Probe Name Ag2925

| | | | Start | SEQ ID |
|---|---|---|---|---|
| Primers | Sequences | Length | Position | No |
| Forward | 5′-ctccctcacctccatctttaac-3′ | 22 | 1191 | 1350 |
| Probe | TET-5′-ccatcttcaccatggacctctggaat-3′- | 26 | 1223 | 1351 |
| | TAMRA | | | |
| Reverse | 5′-atcatgagctccttctcagatg-3′ | 22 | 1265 | 1352 |

TABLE BAB
CNS_neurodegeneration_v1.0
Column A - Rel. Exp.(%) Ag2925, Run 209777392

| Tissue Name | A | Tissue Name | A |
|---|---|---|---|
| AD 1 Hippo | 51.8 | AH3 4624 | 2.3 |
| AD 2 Hippo | 31.9 | AH3 4640 | 10.7 |
| AD 3 Hippo | 27.7 | AD 1 Occipital Ctx | 100.0 |
| AD 4 Hippo | 5.7 | AD 2 Occipital Ctx (Missing) | 5.0 |
| AD 5 Hippo | 49.3 | AD 3 Occipital Ctx | 48.0 |
| AD 6 Hippo | 19.6 | AD 4 Occipital Ctx | 40.3 |
| Control 2 Hippo | 50.3 | AD 5 Occipital Ctx | 28.1 |
| Control 4 Hippo | 7.5 | AD 5 Occipital Ctx | 50.3 |
| Control (Path) 3 Hippo | 9.3 | Control 1 Occipital Ctx | 13.6 |
| AD 1 Temporal Ctx | 100.0 | Control 2 Occipital Ctx | 79.6 |
| AD 2 Temporal Ctx | 45.7 | Control 3 Occipital Ctx | 18.2 |
| AD 3 Temporal Ctx | 11.3 | Control 4 Occipital Ctx | 49.7 |
| AD 4 Temporal Ctx | 24.1 | Control (Path) 1 Occipital Ctx | 55.1 |
| AD 5 Inf Temporal Ctx | 92.7 | Control (Path) 2 Occipital Ctx | 28.1 |
| AD 5 Sup Temporal Ctx | 43.2 | Control (Path) 3 Occipital Ctx | 31.4 |
| AD 6 Inf Temporal Ctx | 29.7 | Control (Path) 4 Occipital Ctx | 13.9 |
| AD 6 Sup Temporal Ctx | 17.6 | Control 1 Parietal Ctx | 25.0 |
| Control 1 Temporal Ctx | 2.1 | Control 2 Parietal Ctx | 52.1 |
| Control 2 Temporal Ctx | 35.8 | Control 3 Parietal Ctx | 34.4 |
| Control 3 Temporal Ctx | 13.6 | Control (Path) 1 Parietal Ctx | 18.2 |
| Control 3 Temporal Ctx | 4.8 | Control (Path) 2 Parietal Ctx | 35.4 |
| AH3 3975 | 16.2 | Control (Path) 3 Parietal Ctx | 2.8 |
| AH3 3954 | 10.0 | Control (Path) 4 Parietal Ctx | 21.5 |

TABLE BAC
Panel 1.3D
Column A - Rel. Exp.(%) Ag2925, Run 158046924

| Tissue Name | A | Tissue Name | A |
|---|---|---|---|
| Liver adenocarcinoma | 0.4 | Kidney (fetal) | 1.2 |
| Pancreas | 0.0 | Renal ca. 786-0 | 0.0 |
| Pancreatic ca. CAPAN 2 | 0.0 | Renal ca. A498 | 0.1 |
| Adrenal gland | 0.3 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.4 | Renal ca. TK-10 | 0.0 |
| Brain (fetal) | 0.3 | Liver | 0.2 |
| Brain (whole) | 19.5 | Liver (fetal) | 3.8 |
| Brain (amygdala) | 10.5 | Liver ca. (hepatoblast) HepG2 | 0.6 |
| Brain (cerebellum) | 4.6 | Lung | 0.2 |
| Brain (hippocampus) | 100.0 | Lung (fetal) | 0.0 |
| Brain (substantia nigra) | 22.2 | Lung ca. (small cell) LX-1 | 0.0 |
| Brain (thalamus) | 45.4 | Lung ca. (small cell) NCI-H69 | 0.0 |
| Cerebral Cortex | 13.0 | Lung ca. (s. cell var.) SHP-77 | 0.0 |
| Spinal cord | 25.9 | Lung ca. (large cell) NCI-H460 | 0.0 |
| glio/astro U87-MG | 0.0 | Lung ca. (non-sm. cell) A549 | 0.8 |
| glio/astro U-118-MG | 0.4 | Lung ca. (non-s. cell) NCI-H23 | 0.0 |
| astrocytoma SW1783 | 0.1 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.1 | Lung ca. (squam.) SW 900 | 0.0 |
| astrocytoma SNB-75 | 0.1 | Lung ca. (squam.) NCI-H596 | 0.0 |
| glioma SNB-19 | 0.0 | Mammary gland | 0.1 |
| glioma U251 | 0.0 | Breast ca.* (pl. ef) MCF-7 | 0.0 |
| glioma SF-295 | 0.0 | Breast ca.* (pl. ef) MDA-MB-231 | 0.5 |
| Heart (Fetal) | 0.1 | Breast ca.* (pl. ef) T47D | 0.0 |
| Heart | 0.0 | Breast ca. BT-549 | 0.3 |
| Skeletal muscle (Fetal) | 0.1 | Breast ca. MDA-N | 0.2 |
| Skeletal muscle | 0.0 | Ovary | 0.1 |
| Bone marrow | 0.0 | Ovarian ca. OVCAR-3 | 0.0 |
| Thymus | 0.0 | Ovarian ca. OVCAR-4 | 0.0 |
| Spleen | 0.1 | Ovarian ca. OVCAR-5 | 0.0 |
| Lymph node | 0.1 | Ovarian ca. OVCAR-8 | 0.3 |
| Colorectal | 0.1 | Ovarian ca. IGROV-1 | 0.0 |
| Stomach | 0.0 | Ovarian ca. (ascites) SK-OV-3 | 0.0 |
| Small intestine | 1.0 | Uterus | 0.0 |
| Colon ca. SW480 | 0.2 | Placenta | 0.4 |
| Colon ca.* SW620 | 84.7 | Prostate | 0.2 |
| (SW480 met) | | | |
| Colon ca. HT29 | 0.2 | Prostate ca.* (bone met) PC-3 | 0.0 |
| Colon ca. HCT-116 | 0.0 | Testis | 0.3 |
| Colon ca. CaCo-2 | 0.2 | Melanoma Hs688(A).T | 0.0 |
| CC Well to Mod Diff | 0.1 | Melanoma* (met) Hs688(B).T | 0.0 |
| (ODO3866) | | | |
| Colon ca. HCC-2998 | 0.7 | Melanoma UACC-62 | 0.1 |
| Gastric ca. (liver met) | 0.0 | Melanoma M14 | 0.0 |
| NCI-N87 | | | |
| Bladder | 0.0 | Melanoma LOXIMVI | 0.0 |
| Trachea | 0.0 | Melanoma* (met) SK-MEL-5 | 0.0 |
| Kidney | 2.6 | Adipose | 0.0 |

TABLE BAD
Panel 2D
Column A - Rel. Exp.(%) Ag2925, Run 158047169

| Tissue Name | A | Tissue Name | A |
|---|---|---|---|
| Normal Colon | 2.6 | Kidney Margin 8120608 | 63.7 |
| CC Well to Mod Diff (ODO3866) | 0.6 | Kidney Cancer 8120613 | 0.0 |
| CC Margin (ODO3866) | 0.5 | Kidney Margin 8120614 | 100.0 |
| CC Gr.2 rectosigmoid (ODO3868) | 0.0 | Kidney Cancer 9010320 | 0.4 |
| CC Margin (ODO3868) | 0.4 | Kidney Margin 9010321 | 14.2 |
| CC Mod Diff (ODO3920) | 2.0 | Normal Uterus | 0.9 |
| CC Margin (ODO3920) | 0.7 | Uterine Cancer 064011 | 0.7 |
| CC Gr.2 ascend colon (ODO3921) | 0.0 | Normal Thyroid | 0.0 |
| CC Margin (ODO3921) | 1.2 | Thyroid Cancer | 0.0 |
| CC from Partial Hepatectomy | 1.5 | Thyroid Cancer A302152 | 0.0 |
| (ODO4309) Mets | | | |
| Liver Margin (ODO4309) | 0.7 | Thyroid Margin A302153 | 0.4 |
| Colon mets to lung (OD04451-01) | 0.0 | Normal Breast | 0.6 |
| Lung Margin (OD04451-02) | 0.9 | Breast Cancer | 0.9 |
| Normal Prostate 6546-1 | 0.6 | Breast Cancer (OD04590-01) | 1.2 |
| Prostate Cancer (OD04410) | 1.2 | Breast Cancer Mets (OD04590-03) | 0.5 |
| Prostate Margin (OD04410) | 1.2 | Breast Cancer Metastasis | 0.9 |
| Prostate Cancer (OD04720-01) | 0.9 | Breast Cancer | 0.0 |
| Prostate Margin (OD04720-02) | 1.1 | Breast Cancer | 0.9 |
| Normal Lung | 0.4 | Breast Cancer 9100266 | 1.4 |
| Lung Met to Muscle (ODO4286) | 0.0 | Breast Margin 9100265 | 0.0 |
| Muscle Margin (ODO4286) | 0.0 | Breast Cancer A209073 | 44.1 |
| Lung Malignant Cancer (OD03126) | 1.4 | Breast Margin A209073 | 1.4 |
| Lung Margin (OD03126) | 0.0 | Normal Liver | 0.0 |
| Lung Cancer (OD04404) | 0.8 | Liver Cancer | 0.0 |
| Lung Margin (OD04404) | 0.0 | Liver Cancer 1025 | 0.6 |
| Lung Cancer (OD04565) | 0.0 | Liver Cancer 1026 | 1.2 |
| Lung Margin (OD04565) | 0.4 | Liver Cancer 6004-T | 0.3 |
| Lung Cancer (OD04237-01) | 0.0 | Liver Tissue 6004-N | 3.3 |
| Lung Margin (OD04237-02) | 0.0 | Liver Cancer 6005-T | 0.3 |
| Ocular Mel Met to Liver (ODO4310) | 0.0 | Liver Tissue 6005-N | 0.4 |
| Liver Margin (ODO4310) | 0.1 | Normal Bladder | 1.2 |
| Melanoma Metastasis | 0.0 | Bladder Cancer | 0.9 |
| Lung Margin (OD04321) | 0.1 | Bladder Cancer | 0.4 |
| Normal Kidney | 48.3 | Bladder Cancer (OD04718-01) | 0.7 |
| Kidney Ca, Nuclear grade 2 (OD04338) | 0.9 | Bladder Normal Adjacent | 0.0 |
| | | (OD04718-03) | |
| Kidney Margin (OD04338) | 3.8 | Normal Ovary | 0.0 |
| Kidney Ca Nuclear grade 1/2 (OD04339) | 0.4 | Ovarian Cancer | 0.1 |
| Kidney Margin (OD04339) | 70.7 | Ovarian Cancer (OD04768-07) | 1.6 |
| Kidney Ca, Clear cell type (OD04340) | 3.0 | Ovary Margin (OD04768-08) | 0.0 |
| Kidney Margin (OD04340) | 11.7 | Normal Stomach | 1.0 |
| Kidney Ca, Nuclear grade 3 (OD04348) | 0.2 | Gastric Cancer 9060358 | 0.0 |
| Kidney Margin (OD04348) | 2.1 | Stomach Margin 9060359 | 0.0 |
| Kidney Cancer (OD04622-01) | 0.0 | Gastric Cancer 9060395 | 0.4 |
| Kidney Margin (OD04622-03) | 2.3 | Stomach Margin 9060394 | 0.7 |
| Kidney Cancer (OD04450-01) | 0.0 | Gastric Cancer 9060397 | 2.4 |
| Kidney Margin (OD04450-03) | 5.0 | Stomach Margin 9060396 | 0.4 |
| Kidney Cancer 8120607 | 0.3 | Gastric Cancer 064005 | 0.6 |

TABLE BAE
Panel 4D
Column A - Rel. Exp.(%) Ag2925, Run 158047348

| Tissue Name | A |
|---|---|
| Secondary Th1 act | 1.7 |
| Secondary Th2 act | 0.0 |
| Secondary Tr1 act | 0.6 |
| Secondary Th1 rest | 0.0 |
| Secondary Th2 rest | 0.8 |
| Secondary Tr1 rest | 0.0 |
| Primary Th1 act | 0.6 |
| Primary Th2 act | 0.7 |
| Primary Tr1 act | 0.6 |
| Primary Th1 rest | 0.8 |
| Primary Th2 rest | 1.9 |
| Primary Tr1 rest | 1.6 |
| CD45RA CD4 lymphocyte act | 0.5 |
| CD45RO CD4 lymphocyte act | 0.6 |
| CD8 lymphocyte act | 0.5 |
| Secondary CD8 lymphocyte rest | 0.3 |
| Secondary CD8 lymphocyte act | 0.0 |
| CD4 lymphocyte none | 0.1 |
| 2ry Th1/Th2/Tr1 anti-CD95 | 1.2 |
| CH11 | |
| LAK cells rest | 1.1 |
| LAK cells IL-2 | 1.1 |
| LAK cells IL-2 + IL-12 | 1.1 |
| LAK cells IL-2 + IFN gamma | 0.0 |
| LAK cells IL-2 + IL-18 | 0.4 |
| LAK cells PMA/ionomycin | 1.4 |
| NK Cells IL-2 rest | 0.7 |
| Two Way MLR 3 day | 2.3 |
| Two Way MLR 5 day | 0.8 |
| Two Way MLR 7 day | 0.5 |
| PBMC rest | 0.0 |
| PBMC PWM | 1.1 |
| PBMC PHA-L | 0.0 |
| Ramos (B cell) none | 0.0 |
| Ramos (B cell) ionomycin | 0.0 |
| B lymphocytes PWM | 0.6 |
| B lymphocytes CD40L and IL-4 | 0.4 |
| EOL-1 dbcAMP | 0.0 |
| EOL-1 dbcAMP | 0.0 |
| PMA/ionomycin | |
| Dendritic cells none | 1.1 |
| Dendritic cells LPS | 1.0 |
| Dendritic cells anti-CD40 | 1.1 |
| Monocytes rest | 0.6 |
| Monocytes LPS | 0.0 |
| Macrophages rest | 2.2 |
| Macrophages LPS | 0.0 |
| HUVEC none | 0.8 |
| HUVEC starved | 1.6 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.6 |
| Lung Microvascular EC none | 0.2 |
| Lung Microvascular EC TNFalpha + IL- | 0.0 |
| 1beta | |
| Microvascular Dermal EC none | 0.0 |
| Microsvasular Dermal EC TNFalpha + IL- | 0.0 |
| 1beta | |
| Bronchial epithelium TNFalpha + IL1beta | 0.0 |
| Small airway epithelium none | 0.0 |
| Small airway epithelium TNFalpha + IL- | 0.0 |
| 1beta | |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL- | 0.0 |
| 1beta | |
| Liver cirrhosis | 2.8 |
| Lupus kidney | 1.2 |
| NCI-H292 none | 0.5 |
| NCI-H292 IL-4 | 0.2 |
| NCI-H292 IL-9 | 1.1 |
| NCI-H292 IL-13 | 0.6 |
| NCI-H292 IFN gamma | 0.6 |
| HPAEC none | 1.3 |
| HPAEC TNF alpha + IL-1 beta | 0.1 |
| Lung fibroblast none | 0.7 |
| Lung fibroblast TNF alpha + IL-1 beta | 0.0 |
| Lung fibroblast IL-4 | 0.3 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 2.9 |
| Colon | 100.0 |
| Lung | 2.1 |
| Thymus | 85.3 |
| Kidney | 2.9 |

CNS_neurodegeneration_v1.0 Summary: Ag2925 This gene was found to be upregulated in the temporal cortex of Alzheimer's disease patients. Therapeutic modulation of this gene, expressed protein and/or use of antibodies or small molecule drugs targeting the gene or gene product are useful for decreasing neuronal death and as a treatment for this disease.

Panel 1.3D Summary: Ag2925 Expression of this gene was brain-specific. Highest expression was detected in the hippocampus (CT=28) a region that degenerates in Alzheimer's disease. Expression of this gene or its protein product is useful for distinguishing brain tissue from non-neural tissue. Therapeutic modulation of this gene, expressed protein and/or use of antibodies or small molecule drugs targeting the gene or gene product are useful in the treatment of neurodegenerative diseases.

Panel 2D Summary: Ag2925 This gene was most highly expressed in a normal kidney sample (CT=28.95). Expression of this gene was lost in the adjacent cancer samples. The loss of expression of this gene or its protein product is useful as a marker for kidney cancer. This gene was also expressed at low levels in breast and bladder cancer samples and was absent or extremely low in normal adjacent tissue. Therapeutic modulation of this gene, expressed protein and/or use of antibodies or small molecule drugs targeting the gene or gene product are useful in the treatment of breast and bladder cancer and as a diagnostic marker for the presence of these cancers.

Panel 4D Summary: Ag2925 Expression of this transcript was almost exclusively restricted to colon and thymus, with highest expression in normal colon (CT=29). This gene was expressed at much lower levels in IBD colon. The protein encoded by this transcript is involved in normal tissue/cellular functions in the kidney and colon. Loss-of-expression of this protein is useful as a diagnostic marker for lupus or IBD.

BB. CG56645-03 and CG56645-04: Sodium Glucose Cotransporter.

Expression of genes CG56645-03 and CG56645-04 were assessed using the primer-probe sets Ag2966 and Ag6497, described in Tables BBA and BBB. Results of the RTQ-PCR runs are shown in Tables BBC, BBD and BBE.

TABLE BBA
Probe Name Ag2966

| | | | Start | SEQ ID |
|---|---|---|---|---|
| Primers | Sequences | Length | Position | No |
| Forward | 5′-agcgggcaactcttcatcta-3′ | 20 | 1388 | 1353 |
| Probe | TET-5′-atgcagtcagtgaccagctccctg-3′- | 24 | 1409 | 1354 |
| | TAMRA | | | |
| Reverse | 5′-caggacaaagactgcagtcact-3′ | 22 | 1441 | 1355 |

TABLE BBB
Probe Name Ag6497

| | | | Start | SEQ ID |
|---|---|---|---|---|
| Primers | Sequences | Length | Position | No |
| Forward | 5′-tcatagtggcactcatcgg-3′ | 19 | 1329 | 1356 |
| Probe | TET-5′-aactcttcatctacatgcagtcagtgac | 30 | 1395 | 1357 |
| | ca-3′-TAMRA | | | |
| Reverse | 5′-ctatcaggcccaggacaaa-3′ | 19 | 1454 | 1358 |

TABLE BBC
Panel 1.3D
Column A - Rel. Exp. (%) Ag2966, Run 160658385
Column B - Rel. Exp. (%) Ag2966, Run 165701959

| Tissue Name | A | B | Tissue Name | A | B |
|---|---|---|---|---|---|
| Liver adenocarcinoma | 0.0 | 0.0 | Kidney (fetal) | 1.0 | 0.0 |
| Pancreas | 0.2 | 1.3 | Renal ca. 786-0 | 0.2 | 0.0 |
| Pancreatic ca. CAPAN 2 | 0.0 | 0.0 | Renal ca. A498 | 0.0 | 0.3 |
| Adrenal gland | 0.2 | 0.0 | Renal ca. RXF 393 | 0.0 | 0.5 |
| Thyroid | 0.3 | 0.0 | Renal ca. ACHN | 0.3 | 0.0 |
| Salivary gland | 0.1 | 0.4 | Renal ca. UO-31 | 0.0 | 0.0 |
| Pituitary gland | 0.0 | 0.0 | Renal ca. TK-10 | 0.0 | 0.0 |
| Brain (fetal) | 0.0 | 0.0 | Liver | 0.6 | 0.9 |
| Brain (whole) | 0.0 | 0.3 | Liver (fetal) | 0.4 | 0.0 |
| Brain (amygdala) | 0.0 | 0.0 | Liver ca. (hepatoblast) HepG2 | 0.3 | 0.0 |
| Brain (cerebellum) | 0.0 | 0.0 | Lung | 0.8 | 0.6 |
| Brain (hippocampus) | 0.5 | 0.3 | Lung (fetal) | 1.4 | 1.4 |
| Brain (substantia nigra) | 0.0 | 0.0 | Lung ca. (small cell) LX-1 | 0.6 | 0.0 |
| Brain (thalamus) | 0.0 | 0.5 | Lung ca. (small cell) NCI-H69 | 0.0 | 0.0 |
| Cerebral Cortex | 0.0 | 0.0 | Lung ca. (s. cell var.) SHP-77 | 0.3 | 0.4 |
| Spinal cord | 0.2 | 0.2 | Lung ca. (large cell) NCI-H460 | 0.2 | 0.0 |
| glio/astro U87-MG | 0.0 | 0.0 | Lung ca. (non-sm. cell) A549 | 0.8 | 0.7 |
| glio/astro U-118-MG | 0.0 | 0.0 | Lung ca. (non-s. cell) NCI-H23 | 0.0 | 0.0 |
| astrocytoma SW1783 | 0.0 | 0.0 | Lung ca. (non-s. cell) HOP-62 | 1.6 | 2.2 |
| neuro*; met SK-N-AS | 0.0 | 0.4 | Lung ca. (non-s. cl) NCI-H522 | 0.5 | 0.0 |
| astrocytoma SF-539 | 0.0 | 0.0 | Lung ca. (squam.) SW 900 | 0.0 | 0.0 |
| astrocytoma SNB-75 | 0.0 | 0.0 | Lung ca. (squam.) NCI-H596 | 0.0 | 0.0 |
| glioma SNB-19 | 0.0 | 0.4 | Mammary gland | 0.3 | 0.7 |
| glioma U251 | 0.0 | 0.4 | Breast ca.* (pl. ef) MCF-7 | 0.2 | 0.0 |
| glioma SF-295 | 0.0 | 0.0 | Breast ca.* (pl. ef) MDA-MB-231 | 0.0 | 0.0 |
| Heart (Fetal) | 0.0 | 0.0 | Breast ca.* (pl. ef) T47D | 0.0 | 0.0 |
| Heart | 0.0 | 0.0 | Breast ca. BT-549 | 0.2 | 0.0 |
| Skeletal muscle (Fetal) | 3.9 | 0.3 | Breast ca. MDA-N | 0.0 | 0.0 |
| Skeletal muscle | 0.0 | 0.0 | Ovary | 0.5 | 0.0 |
| Bone marrow | 6.9 | 2.5 | Ovarian ca. OVCAR-3 | 0.0 | 0.0 |
| Thymus | 1.9 | 0.9 | Ovarian ca. OVCAR-4 | 0.0 | 0.4 |
| Spleen | 4.2 | 1.0 | Ovarian ca. OVCAR-5 | 0.3 | 0.0 |
| Lymph node | 3.0 | 5.4 | Ovarian ca. OVCAR-8 | 0.0 | 0.0 |
| Colorectal | 0.5 | 0.0 | Ovarian ca. IGROV-1 | 0.0 | 0.0 |
| Stomach | 0.9 | 0.0 | Ovarian ca. (ascites) SK-OV-3 | 0.0 | 0.0 |
| Small intestine | 1.5 | 0.5 | Uterus | 0.0 | 0.5 |
| Colon ca. SW480 | 0.8 | 0.0 | Placenta | 0.0 | 0.0 |
| Colon ca.* SW620 (SW480 met) | 0.0 | 0.0 | Prostate | 0.0 | 0.0 |
| Colon ca. HT29 | 0.0 | 0.0 | Prostate ca.* (bone met) PC-3 | 0.0 | 0.0 |
| Colon ca. HCT-116 | 0.0 | 0.0 | Testis | 1.6 | 0.8 |
| Colon ca. CaCo-2 | 0.0 | 0.0 | Melanoma Hs688(A).T | 0.0 | 0.0 |
| CC Well to Mod Diff (ODO3866) | 0.3 | 0.3 | Melanoma* (met) Hs688(B).T | 0.0 | 0.0 |
| Colon ca. HCC-2998 | 0.3 | 0.0 | Melanoma UACC-62 | 0.0 | 0.0 |
| Gastric ca. (liver met) NCI-N87 | 0.2 | 0.7 | Melanoma M14 | 0.0 | 0.0 |
| Bladder | 0.2 | 0.3 | Melanoma LOX IMVI | 0.0 | 0.0 |
| Trachea | 0.6 | 0.0 | Melanoma* (met) SK-MEL-5 | 0.0 | 0.0 |
| Kidney | 100.0 | 100.0 | Adipose | 0.1 | 0.6 |

TABLE BBD
Panel 2D
Column A - Rel. Exp.(%) Ag2966, Run 160658389

| Tissue Name | A | Tissue Name | A |
|---|---|---|---|
| Normal Colon | 0.2 | Kidney Margin 8120608 | 91.4 |
| CC Well to Mod Diff (ODO3866) | 0.1 | Kidney Cancer 8120613 | 0.1 |
| CC Margin (ODO3866) | 0.3 | Kidney Margin 8120614 | 100.0 |
| CC Gr.2 rectosigmoid (ODO3868) | 0.3 | Kidney Cancer 9010320 | 0.4 |
| CC Margin (ODO3868) | 0.1 | Kidney Margin 9010321 | 66.9 |
| CC Mod Diff (ODO3920) | 0.6 | Normal Uterus | 0.2 |
| CC Margin (ODO3920) | 0.4 | Uterine Cancer 064011 | 0.4 |
| CC Gr.2 ascend colon (ODO3921) | 0.4 | Normal Thyroid | 0.1 |
| CC Margin (ODO3921) | 0.3 | Thyroid Cancer | 0.0 |
| CC from Partial Hepatectomy | 0.4 | Thyroid Cancer A302152 | 0.0 |
| (ODO4309) Mets | | | |
| Liver Margin (ODO4309) | 0.2 | Thyroid Margin A302153 | 0.5 |
| Colon mets to lung (OD04451-01) | 0.5 | Normal Breast | 0.8 |
| Lung Margin (OD04451-02) | 0.1 | Breast Cancer | 0.3 |
| Normal Prostate 6546-1 | 0.0 | Breast Cancer (OD04590-01) | 0.8 |
| Prostate Cancer (OD04410) | 0.2 | Breast Cancer Mets (OD04590-03) | 0.8 |
| Prostate Margin (OD04410) | 0.0 | Breast Cancer Metastasis | 0.9 |
| Prostate Cancer (OD04720-01) | 0.1 | Breast Cancer | 0.2 |
| Prostate Margin (OD04720-02) | 0.2 | Breast Cancer | 0.4 |
| Normal Lung | 1.5 | Breast Cancer 9100266 | 0.1 |
| Lung Met to Muscle (ODO4286) | 0.2 | Breast Margin 9100265 | 0.0 |
| Muscle Margin (ODO4286) | 0.0 | Breast Cancer A209073 | 0.2 |
| Lung Malignant Cancer (OD03126) | 0.2 | Breast Margin A209073 | 0.3 |
| Lung Margin (OD03126) | 0.4 | Normal Liver | 0.1 |
| Lung Cancer (OD04404) | 0.4 | Liver Cancer | 0.1 |
| Lung Margin (OD04404) | 0.4 | Liver Cancer 1025 | 0.2 |
| Lung Cancer (OD04565) | 0.2 | Liver Cancer 1026 | 0.0 |
| Lung Margin (OD04565) | 0.4 | Liver Cancer 6004-T | 0.1 |
| Lung Cancer (OD04237-01) | 0.1 | Liver Tissue 6004-N | 0.1 |
| Lung Margin (OD04237-02) | 0.0 | Liver Cancer 6005-T | 0.0 |
| Ocular Mel Met to Liver (ODO4310) | 1.8 | Liver Tissue 6005-N | 0.0 |
| Liver Margin (ODO4310) | 0.0 | Normal Bladder | 0.2 |
| Melanoma Metastasis | 0.1 | Bladder Cancer | 0.1 |
| Lung Margin (OD04321) | 0.4 | Bladder Cancer | 0.1 |
| Normal Kidney | 29.9 | Bladder Cancer (0D04718-01) | 0.1 |
| Kidney Ca, Nuclear grade 2 (OD04338) | 9.4 | Bladder Normal Adjacent | |
| | | (OD04718-03) | 0.1 |
| Kidney Margin (OD04338) | 26.2 | Normal Ovary | 0.0 |
| Kidney Ca Nuclear grade 1/2 (OD04339) | 1.3 | Ovarian Cancer | 0.1 |
| Kidney Margin (OD04339) | 75.8 | Ovarian Cancer (OD04768-07) | 0.0 |
| Kidney Ca, Clear cell type (OD04340) | 29.5 | Ovary Margin (OD04768-08) | 0.0 |
| Kidney Margin (OD04340) | 27.5 | Normal Stomach | 0.1 |
| Kidney Ca, Nuclear grade 3 (OD04348) | 0.7 | Gastric Cancer 9060358 | 0.1 |
| Kidney Margin (OD04348) | 11.4 | Stomach Margin 9060359 | 0.1 |
| Kidney Cancer (OD04622-01) | 0.9 | Gastric Cancer 9060395 | 0.1 |
| Kidney Margin (OD04622-03) | 9.6 | Stomach Margin 9060394 | 0.1 |
| Kidney Cancer (OD04450-01) | 0.8 | Gastric Cancer 9060397 | 0.1 |
| Kidney Margin (OD04450-03) | 8.2 | Stomach Margin 9060396 | 0.0 |
| Kidney Cancer 8120607 | 0.6 | Gastric Cancer 064005 | 0.3 |

TABLE BBE
Panel 4D
Column A - Rel. Exp.(%) Ag2966, Run 160660646

| Tissue Name | A | Tissue Name | A |
|---|---|---|---|
| Secondary Th1 act | 0.0 | HUVEC IL-1beta | 0.3 |
| Secondary Th2 act | 0.0 | HUVEC IFN gamma | 0.9 |
| Secondary Tr1 act | 0.7 | HUVEC TNF alpha + IFN gamma | 0.3 |
| Secondary Th1 rest | 0.2 | HUVEC TNF alpha + IL4 | 0.5 |
| Secondary Th2 rest | 1.6 | HUVEC IL-11 | 0.8 |
| Secondary Tr1 rest | 1.5 | Lung Microvascular EC none | 2.0 |
| Primary Th1 act | 0.3 | Lung Microvascular EC TNFalpha + IL- | 0.8 |
| | | 1beta | |
| Primary Th2 act | 0.0 | Microvascular Dermal EC none | 1.9 |
| Primary Tr1 act | 0.3 | Microsvasular Dermal EC TNFalpha + IL- | 1.7 |
| | | 1beta | |
| Primary Th1 rest | 4.8 | Bronchial epithelium TNFalpha + IL1beta | 0.0 |
| Primary Th2 rest | 2.1 | Small airway epithelium none | 0.0 |
| Primary Tr1 rest | 3.4 | Small airway epithelium TNFalpha + IL- | 0.3 |
| | | 1beta | |
| CD45RA CD4 lymphocyte act | 0.6 | Coronery artery SMC rest | 0.0 |
| CD45RO CD4 lymphocyte act | 1.1 | Coronery artery SMC TNFalpha + IL-1beta | 0.3 |
| CD8 lymphocyte act | 0.3 | Astrocytes rest | 0.0 |
| Secondary CD8 lymphocyte rest | 1.3 | Astrocytes TNFalpha + IL-1beta | 0.3 |
| Secondary CD8 lymphocyte act | 0.0 | KU-812 (Basophil) rest | 0.3 |
| CD4 lymphocyte none | 1.7 | KU-812 (Basophil) PMA/ionomycin | 0.0 |
| 2ry Th1/Th2/Tr1 anti-CD95 | 0.8 | CCD1106 (Keratinocytes) none | 0.1 |
| CH11 | | | |
| LAK cells rest | 2.1 | CCD1106 (Keratinocytes) TNFalpha + IL- | 0.0 |
| | | 1beta | |
| LAK cells IL-2 | 0.4 | Liver cirrhosis | 0.4 |
| LAK cells IL-2 + IL-12 | 0.3 | Lupus kidney | 1.0 |
| LAK cells IL-2 + IFN gamma | 0.6 | NCI-H292 none | 0.6 |
| LAK cells IL-2 + IL-18 | 0.6 | NCI-H292 IL-4 | 0.0 |
| LAK cells PMA/ionomycin | 0.0 | NCI-H292 IL-9 | 0.0 |
| NK Cells IL-2 rest | 0.6 | NCI-H292 IL-13 | 0.0 |
| Two Way MLR 3 day | 1.1 | NCI-H292 IFN gamma | 0.0 |
| Two Way MLR 5 day | 1.0 | HPAEC none | 0.6 |
| Two Way MLR 7 day | 0.5 | HPAEC TNF alpha + IL-1 beta | 0.0 |
| PBMC rest | 0.6 | Lung fibroblast none | 0.0 |
| PBMC PWM | 1.1 | Lung fibroblast TNF alpha + IL-1 beta | 0.0 |
| PBMC PHA-L | 0.2 | Lung fibroblast IL-4 | 0.0 |
| Ramos (B cell) none | 1.7 | Lung fibroblast IL-9 | 0.0 |
| Ramos (B cell) ionomycin | 1.1 | Lung fibroblast IL-13 | 0.0 |
| B lymphocytes PWM | 1.4 | Lung fibroblast IFN gamma | 0.2 |
| B lymphocytes CD40L and IL-4 | 4.7 | Dermal fibroblast CCD1070 rest | 0.0 |
| EOL-1 dbcAMP | 0.0 | Dermal fibroblast CCD1070 TNF alpha | 1.4 |
| EOL-1 dbcAMP | 0.1 | Dermal fibroblast CCD1070 IL-1 beta | 0.0 |
| PMA/ionomycin | | | |
| Dendritic cells none | 0.5 | Dermal fibroblast IFN gamma | 0.0 |
| Dendritic cells LPS | 0.0 | Dermal fibroblast IL-4 | 0.0 |
| Dendritic cells anti-CD40 | 0.5 | IBD Colitis 2 | 0.2 |
| Monocytes rest | 1.2 | IBD Crohn's | 0.0 |
| Monocytes LPS | 0.0 | Colon | 4.9 |
| Macrophages rest | 0.2 | Lung | 3.1 |
| Macrophages LPS | 0.0 | Thymus | 100.0 |
| HUVEC none | 0.5 | Kidney | 4.6 |
| HUVEC starved | 0.3 | | |

Panel 1.3D Summary: Ag2966 Expression of this gene, a sodium-glucose cotransporter homolog, was limited to the kidney (CTs=29). This restricted expression was in agreement with published data, where secondary active transport of glucose in the kidney is mediated by sodium glucose cotransporter. (Bissonnette P. J Physiol 1999 Oct. 15; 520 Pt 2:359-71). Expression of this gene or its protein product is useful as a marker of kidney tissue. Therapeutic modulation of this gene, expressed protein and/or use of antibodies or small molecule drugs targeting the gene or gene product are useful in the treatment of diseases that affect the kidney, including diabetes.

Panel 2D Summary: Ag2966 Expression of this gene was predominantly limited to the kidney. The expression was downregulated in kidney cancer samples. Therapeutic modulation of this gene, expressed protein and/or use of antibodies or small molecule drugs targeting the gene or gene product are useful in the treatment of diseases that affect the kidney, including kidney cancer.

Panel 4D Summary: Ag2966 Expression of this gene was predominantly found in normal tissue from thymus, lung, colon and kidney. This expression profile indicates that the protein product is involved in glucose transport and normal homeostasis in these tissues. Therapeutic modulation of this gene, expressed protein and/or use of antibodies or small molecule drugs targeting the gene or gene product are useful for maintaining or restoring normal function to these organs during inflammation.

BC. CG56667-01: GPCR.

Expression of gene CG56667-01 was assessed using the primer-probe set Ag2973, described in Table BCA. Results of the RTQ-PCR runs are shown in Table BCB.

TABLE BCA
Probe Name Ag2973

| | | | Start | SEQ ID |
|---|---|---|---|---|
| Primers | Sequences | Length | Position | No |
| Forward | 5′-gctgtgtggctcaagtctattt-3′ | 22 | 287 | 1359 |
| Probe | TET-5′-ttctctgcctttgcatctgctgagct-3′- | 26 | 310 | 1360 |
| | TAMRA | | | |
| Reverse | 5′-agcggtcataagacatgacagt-3′ | 22 | 346 | 1361 |

TABLE BCB
Panel 4D
Column A - Rel. Exp.(%) Ag2973, Run 164329850

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 11.7 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 0.0 |
| Primary Tr1 act | 0.0 | Microsvasular Dermal EC TNFalpha + IL- | 0.0 |
| | | 1beta | |
| Primary Th1 rest | 0.0 | Bronchial epithelium TNFalpha + IL1beta | 0.0 |
| Primary Th2 rest | 6.0 | Small airway epithelium none | 0.0 |
| Primary Tr1 rest | 0.0 | Small airway epithelium TNFalpha + IL- | 0.0 |
| | | 1beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL-1beta | 0.0 |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 100.0 |
| LAK cells IL-2 + IL-12 | 0.0 | Lupus kidney | 9.7 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 beta | 0.0 |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1 beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 8.7 |
| Macrophages rest | 0.0 | Lung | 6.6 |
| Macrophages LPS | 0.0 | Thymus | 13.9 |
| HUVEC none | 0.0 | Kidney | 0.0 |
| HUVEC starved | 0.0 | | |

Panel 4D Summary: Ag2973 Significant expression of the CG56667-01 gene was detected in a liver cirrhosis sample (CT=32.7). Expression of this gene was not detected in normal liver in Panel 1.3D, suggesting that its expression is unique to liver cirrhosis. Therapeutic modulation of this gene, expressed protein and/or use of antibodies or small molecule drugs targeting the gene or gene product are useful in the treatment of fibrosis that occurs in liver cirrhosis. Expression of this gene or expressed protein is useful in the diagnosis of liver cirrhosis.

BD. CG56868-01: ADAM7.

Expression of gene CG56868-01 was assessed using the primer-probe sets Ag1322, Ag1322b, Ag2071 and Ag2098, described in Tables BDA, BDB, BDC and BDD. Results of the RTQ-PCR runs are shown in Table BDE.

TABLE BDA
Probe Name Ag1322

| | | | Start | SEQ ID |
|---|---|---|---|---|
| Primers | Sequences | Length | Position | No |
| Forward | 5′-ggtatgtgcctgccctattatt-3′ | 22 | 972 | 1362 |
| Probe | TET-5′-ccaccagtatcattaaggatcttttacc | 30 | 994 | 1363 |
| | tg-3′-TAMRA | | | |
| Reverse | 5′-gccattctgtttgcaattatgt-3′ | 22 | 1030 | 1364 |

TABLE BDB
Probe Name Ag1322b

| | | | Start | SEQ ID |
|---|---|---|---|---|
| Primers | Sequences | Length | Position | No |
| Forward | 5′-ggtatgtgcctgccctattatt-3′ | 22 | 972 | 1365 |
| Probe | TET-5′-ccaccagtatcattaaggatcttttacc | 30 | 994 | 1366 |
| | tg-3′-TAMRA | | | |
| Reverse | 5′-gccattctgtttgcaattatgt-3′ | 22 | 1030 | 1367 |

TABLE BDC
Probe Name Ag2071

| | | | Start | SEQ ID |
|---|---|---|---|---|
| Primers | Sequences | Length | Position | No |
| Forward | 5′-tgccagaaattcatttcctaaa-3′ | 22 | 2236 | 1368 |
| Probe | TET-5′-ccttggaaagcctgcccactagtttt-3′- | 26 | 2275 | 1369 |
| | TAMRA | | | |
| Reverse | 5′-agtgtgatgtagtggggacttg-3′ | 22 | 2302 | 1370 |

TABLE BDD
Probe Name Ag2098

| | | | Start | SEQ ID |
|---|---|---|---|---|
| Primers | Sequences | Length | Position | No |
| Forward | 5′-tgccagaaattcatttcctaaa-3′ | 22 | 2236 | 1371 |
| Probe | TET-5′-ccttggaaagcctgcccactagtttt-3′- | 26 | 2275 | 1372 |
| | TAMRA | | | |
| Reverse | 5′-agtgtgatgtagtggggacttg-3′ | 22 | 2302 | 1373 |

TABLE BDE
Panel 1.2
Column A - Rel. Exp.(%) Ag1322, Run 133804727

| Tissue Name | A | Tissue Name | A |
|---|---|---|---|
| Endothelial cells | 0.0 | Renal ca. 786-0 | 0.0 |
| Heart (Fetal) | 0.0 | Renal ca. A498 | 0.0 |
| Pancreas | 0.2 | Renal ca. RXF 393 | 0.0 |
| Pancreatic ca. CAPAN 2 | 0.0 | Renal ca. ACHN | 0.0 |
| Adrenal gland | 0.2 | Renal ca. UO-31 | 0.0 |
| Thyroid | 0.0 | Renal ca. TK-10 | 0.0 |
| Salivary gland | 0.1 | Liver | 0.0 |
| Pituitary gland | 0.0 | Liver (fetal) | 0.2 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.1 |
| Brain (cerebellum) | 0.0 | Lung ca. (small cell) LX-1 | 0.0 |
| Brain (hippocampus) | 0.0 | Lung ca. (small cell) NCI-H69 | 0.0 |
| Brain (thalamus) | 0.0 | Lung ca. (s. cell var.) SHP-77 | 0.0 |
| Cerebral Cortex | 0.0 | Lung ca. (large cell)NCI-H460 | 0.0 |
| Spinal cord | 0.1 | Lung ca. (non-sm. cell) A549 | 0.0 |
| glio/astro U87-MG | 0.0 | Lung ca. (non-s. cell) NCI-H23 | 0.0 |
| glio/astro U-118-MG | 0.0 | Lung ca. (non-s. cell) HOP-62 | 0.0 |
| astrocytoma SW1783 | 0.0 | Lung ca. (non-s. cl) NCI-H522 | 0.0 |
| neuro*; met SK-N-AS | 0.0 | Lung ca. (squam.) SW 900 | 0.0 |
| astrocytoma SF-539 | 0.0 | Lung ca. (squam.) NCI-H596 | 0.0 |
| astrocytoma SNB-75 | 0.0 | Mammary gland | 0.0 |
| glioma SNB-19 | 0.0 | Breast ca.* (pl. ef) MCF-7 | 0.0 |
| glioma U251 | 0.1 | Breast ca.* (pl. ef) MDA-MB-231 | 0.0 |
| glioma SF-295 | 0.0 | Breast ca.* (pl. ef) T47D | 0.0 |
| Heart | 0.0 | Breast ca. BT-549 | 0.0 |
| Skeletal muscle | 0.0 | Breast ca. MDA-N | 0.0 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.1 | Ovarian ca. OVCAR-5 | 0.0 |
| Colorectal | 0.0 | Ovarian ca. OVCAR-8 | 0.0 |
| Stomach | 0.1 | Ovarian ca. IGROV-1 | 0.0 |
| Small intestine | 0.0 | Ovarian ca. (ascites) SK-OV-3 | 0.0 |
| Colon ca. SW480 | 0.0 | Uterus | 0.0 |
| Colon ca.* SW620 (SW480 met) | 0.0 | Placenta | 0.0 |
| Colon ca. HT29 | 0.0 | Prostate | 2.1 |
| Colon ca. HCT-116 | 0.0 | Prostate ca.* (bone met) PC-3 | 0.0 |
| Colon ca. CaCo-2 | 0.0 | Testis | 100.0 |
| CC Well to Mod Diff (ODO3866) | 0.0 | Melanoma Hs688(A).T | 0.0 |
| Colon ca. HCC-2998 | 0.0 | Melanoma* (met) Hs688(B).T | 0.0 |
| Gastric ca. (liver met) NCI-N87 | 0.0 | Melanoma UACC-62 | 0.0 |
| Bladder | 0.1 | Melanoma M14 | 0.0 |
| Trachea | 0.0 | Melanoma LOX IMVI | 0.0 |
| Kidney | 0.0 | Melanoma* (met) SK-MEL-5 | 0.0 |
| Kidney (fetal) | 0.0 | | |

Panel 1.2 Summary: Ag1322 Expression of this gene was highest in testis (CT value=29). Low expression was also seen in prostate (CT value=34.6). The gene or encoded protein is useful as a marker for these tissues. This gene encodes a protein with homology to ADAM proteins, which are membrane disintegrin-metalloproteases. The expression of several other ADAM proteins has been shown to be testis-specific and these proteins are thought to play a role in fertilization (Hooft van Huijsduijnen R. (1998) ADAM 20 and 21; two novel human testis-specific membrane metalloproteases with similarity to fertilin-alpha. Gene 206: 273-282). Therapeutic modulation of this gene, expressed protein and/or use of antibodies or small molecule drugs targeting the gene or gene product are useful in the treatment of diseases of the prostate and testis, including infertility.

BE. CG56870-01: NDR3.

Expression of genes CG56870-01 and CG56870-06 was assessed using the primer-probe set Ag2075, described in Table BEA. Results of the RTQ-PCR runs are shown in Tables BEB, BEC, BED and BEE.

TABLE BEA
Probe Name Ag2075

| | | | | |
|---|---|---|---|---|
| | | | Start | SEQ ID |
| Primers | Sequences | Length | Position | No |

| | | | | |
|---|---|---|---|---|
| Forward | 5′-catggatgaacttcaggatgtt-3′ | 22 | 70 | 1374 |
| Probe | TET-5′-cagctcacagagatcaaaccacttct-3′- | 26 | 92 | 1375 |
| | TAMRA | | | |
| Reverse | 5′-tgacagtcaaagtcctggaagt-3′ | 22 | 141 | 1376 |

TABLE BEB
Panel 1.3D
Column A - Rel. Exp.(%) Ag2075, Run 152355202

| Tissue Name | A | Tissue Name | A |
|---|---|---|---|
| Liver adenocarcinoma | 11.9 | Kidney (fetal) | 1.4 |
| Pancreas | 0.8 | Renal ca. 786-0 | 3.5 |
| Pancreatic ca. CAPAN 2 | 1.6 | Renal ca. A498 | 12.9 |
| Adrenal gland | 2.4 | Renal ca. RXF 393 | 1.5 |
| Thyroid | 1.8 | Renal ca. ACHN | 1.7 |
| Salivary gland | 1.2 | Renal ca. UO-31 | 6.8 |
| Pituitary gland | 2.4 | Renal ca. TK-10 | 2.8 |
| Brain (fetal) | 3.2 | Liver | 0.4 |
| Brain (whole) | 25.2 | Liver (fetal) | 0.8 |
| Brain (amygdala) | 14.1 | Liver ca. (hepatoblast) HepG2 | 6.7 |
| Brain (cerebellum) | 10.8 | Lung | 2.1 |
| Brain (hippocampus) | 39.8 | Lung (fetal) | 3.2 |
| Brain (substantia nigra) | 2.7 | Lung ca. (small cell) LX-1 | 4.1 |
| Brain (thalamus) | 12.4 | Lung ca. (small cell) NCI-H69 | 3.8 |
| Cerebral Cortex | 100.0 | Lung ca. (s. cell var.) SHP-77 | 3.8 |
| Spinal cord | 3.5 | Lung ca. (large cell)NCI-H460 | 0.6 |
| glio/astro U87-MG | 6.4 | Lung ca. (non-sm. cell) A549 | 3.0 |
| glio/astro U-118-MG | 10.4 | Lung ca. (non-s. cell) NCI-H23 | 7.7 |
| astrocytoma SW1783 | 5.1 | Lung ca. (non-s. cell) HOP-62 | 3.1 |
| neuro*; met SK-N-AS | 6.2 | Lung ca. (non-s. cl) NCI-H522 | 6.3 |
| astrocytoma SF-539 | 5.4 | Lung ca. (squam.) SW 900 | 1.4 |
| astrocytoma SNB-75 | 6.7 | Lung ca. (squam.) NCI-H596 | 1.4 |
| glioma SNB-19 | 3.1 | Mammary gland | 2.4 |
| glioma U251 | 2.0 | Breast ca.* (pl. ef) MCF-7 | 1.8 |
| glioma SF-295 | 3.4 | Breast ca.* (pl. ef) MDA-MB-231 | 11.6 |
| Heart (Fetal) | 6.7 | Breast ca.* (pl. ef) T47D | 9.0 |
| Heart | 1.4 | Breast ca. BT-549 | 4.8 |
| Skeletal muscle (Fetal) | 18.0 | Breast ca. MDA-N | 4.2 |
| Skeletal muscle | 0.7 | Ovary | 13.4 |
| Bone marrow | 0.9 | Ovarian ca. OVCAR-3 | 1.8 |
| Thymus | 0.9 | Ovarian ca. OVCAR-4 | 1.6 |
| Spleen | 3.7 | Ovarian ca. OVCAR-5 | 1.8 |
| Lymph node | 1.9 | Ovarian ca. OVCAR-8 | 4.9 |
| Colorectal | 3.3 | Ovarian ca. IGROV-1 | 1.6 |
| Stomach | 2.7 | Ovarian ca. (ascites) SK-OV-3 | 5.8 |
| Small intestine | 2.5 | Uterus | 2.4 |
| Colon ca. SW480 | 10.4 | Placenta | 1.2 |
| Colon ca.* SW620 (SW480 met) | 3.7 | Prostate | 6.0 |
| Colon ca. HT29 | 4.6 | Prostate ca.* (bone met) PC-3 | 4.5 |
| Colon ca. HCT-116 | 3.6 | Testis | 1.7 |
| Colon ca. CaCo-2 | 10.3 | Melanoma Hs688(A).T | 3.8 |
| CC Well to Mod Diff (ODO3866) | 3.2 | Melanoma* (met) Hs688(B).T | 4.4 |
| Colon ca. HCC-2998 | 2.6 | Melanoma UACC-62 | 1.4 |
| Gastric ca. (liver met) NCI-N87 | 3.1 | Melanoma M14 | 1.8 |
| Bladder | 0.8 | Melanoma LOX IMVI | 3.3 |
| Trachea | 2.0 | Melanoma* (met) SK-MEL-5 | 2.4 |
| Kidney | 1.3 | Adipose | 1.2 |

TABLE BEC
Panel 2.2
Column A - Rel. Exp.(%) Ag2075, Run 174255357

| Tissue Name | A | Tissue Name | A |
|---|---|---|---|
| Normal Colon | 27.7 | Kidney Margin (OD04348) | 46.3 |
| Colon cancer (OD06064) | 52.9 | Kidney malignant cancer | 19.2 |
| | | (OD06204B) | |
| Colon Margin (OD06064) | 30.1 | Kidney normal adjacent tissue | 14.3 |
| | | (OD06204E) | |
| Colon cancer (OD06159) | 5.5 | Kidney Cancer (OD04450-01) | 66.0 |
| Colon Margin (OD06159) | 21.0 | Kidney Margin (OD04450-03) | 19.8 |
| Colon cancer (OD06297-04) | 22.2 | Kidney Cancer 8120613 | 3.0 |
| Colon Margin (OD06297-05) | 41.8 | Kidney Margin 8120614 | 11.0 |
| CC Gr.2 ascend colon (ODO3921) | 4.0 | Kidney Cancer 9010320 | 6.8 |
| CC Margin (ODO3921) | 6.7 | Kidney Margin 9010321 | 9.7 |
| Colon cancer metastasis (OD06104) | 11.1 | Kidney Cancer 8120607 | 35.6 |
| Lung Margin (OD06104) | 42.6 | Kidney Margin 8120608 | 13.7 |
| Colon mets to lung (OD04451-01) | 117.8 | Normal Uterus | 59.5 |
| Lung Margin (OD04451-02) | 9.3 | Uterine Cancer 064011 | 9.7 |
| Normal Prostate | 51.4 | Normal Thyroid | 8.8 |
| Prostate Cancer (OD04410) | 23.0 | Thyroid Cancer | 11.2 |
| Prostate Margin (OD04410) | 19.3 | Thyroid Cancer A302152 | 17.9 |
| Normal Ovary | 22.8 | Thyroid Margin A302153 | 5.5 |
| Ovarian cancer (OD06283-03) | 9.9 | Normal Breast | 33.2 |
| Ovarian Margin (OD06283-07) | 17.1 | Breast Cancer | 8.5 |
| Ovarian Cancer | 18.0 | Breast Cancer | 36.1 |
| Ovarian cancer (OD06145) | 6.6 | Breast Cancer (OD04590-01) | 18.4 |
| Ovarian Margin (OD06145) | 12.5 | Breast Cancer Mets (OD04590-03) | 31.9 |
| Ovarian cancer (OD06455-03) | 14.7 | Breast Cancer Metastasis | 45.4 |
| Ovarian Margin (OD06455-07) | 21.8 | Breast Cancer | 11.5 |
| Normal Lung | 21.9 | Breast Cancer 9100266 | 20.9 |
| Invasive poor diff. lung adeno | 17.6 | Breast Margin 9100265 | 35.1 |
| (ODO4945-01 | | | |
| Lung Margin (ODO4945-03) | 12.2 | Breast Cancer A209073 | 9.7 |
| Lung Malignant Cancer (OD03126) | 8.7 | Breast Margin A209073 | 22.2 |
| Lung Margin (OD03126) | 7.4 | Breast cancer (OD06083) | 100.0 |
| Lung Cancer (OD05014A) | 9.9 | Breast cancer node metastasis | 63.7 |
| | | (OD06083) | |
| Lung Margin (OD05014B) | 21.8 | Normal Liver | 9.9 |
| Lung cancer (OD06081) | 5.1 | Liver Cancer 1026 | 5.6 |
| Lung Margin (OD06081) | 7.9 | Liver Cancer 1025 | 13.5 |
| Lung Cancer (OD04237-01) | 17.4 | Liver Cancer 6004-T | 4.8 |
| Lung Margin (OD04237-02) | 24.0 | Liver Tissue 6004-N | 9.3 |
| Ocular Mel Met to Liver (ODO4310) | 9.7 | Liver Cancer 6005-T | 15.7 |
| Liver Margin (ODO4310) | 4.6 | Liver Tissue 6005-N | 20.4 |
| Melanoma Metastasis | 19.8 | Liver Cancer | 10.7 |
| Lung Margin (OD04321) | 21.6 | Normal Bladder | 8.0 |
| Normal Kidney | 11.0 | Bladder Cancer | 10.5 |
| Kidney Ca, Nuclear grade 2 | 37.6 | Bladder Cancer | 17.3 |
| (OD04338) | | | |
| Kidney Margin (OD04338) | 22.1 | Normal Stomach | 37.9 |
| Kidney Ca Nuclear grade 1/2 | 21.6 | Gastric Cancer 9060397 | 11.1 |
| (OD04339) | | | |
| Kidney Margin (OD04339) | 12.9 | Stomach Margin 9060396 | 20.6 |
| Kidney Ca, Clear cell type (OD04340) | 6.5 | Gastric Cancer 9060395 | 22.7 |
| Kidney Margin (OD04340) | 18.4 | Stomach Margin 9060394 | 36.1 |
| Kidney Ca, Nuclear grade 3 | 12.9 | Gastric Cancer 064005 | 8.1 |
| (OD04348) | | | |

TABLE BED
Panel 3D
Column A - Rel. Exp.(%) Ag2075, Run 164750734

| Tissue Name | A | Tissue Name | A |
|---|---|---|---|
| 94905 Daoy | 6.7 | 94954 Ca Ski Cervical epidermoid | 50.3 |
| Medulloblastoma/Cerebellum | | carcinoma (metastasis | |
| 94906 TE671 | 9.0 | 94955 ES-2 Ovarian clear cell | 13.9 |
| Medulloblastom/Cerebellum | | carcinoma | |
| 94907 D283 Med | 35.4 | 94957 Ramos Stimulated with | 2.7 |
| Medulloblastoma/Cerebellum | | PMA/ionomycin 6 h | |
| 94908 PFSK-1 Primitive | 15.4 | 94958 Ramos Stimulated with | 3.3 |
| Neuroectodermal/Cerebellum | | PMA/ionomycin 14 h | |
| 94909 XF-498 CNS | 4.4 | 94962 MEG-01 Chronic | 3.4 |
| | | myelogenous leukemia | |
| | | (megokaryoblast) | |
| 94910 SNB-78 CNS/glioma | 23.3 | 94963 Raji Burkitt's lymphoma | 3.7 |
| 94911 SF-268 CNS/glioblastoma | 13.6 | 94964 Daudi Burkitt's lymphoma | 6.5 |
| 94912 T98G Glioblastoma | 18.6 | 94965 U266 B-cell | 8.4 |
| | | plasmacytoma/myeloma | 8.4 |
| 96776 SK-N-SH Neuroblastoma | 17.0 | 94968 CA46 Burkitt's lymphoma | 7.2 |
| (metastasis) | | | |
| 94913 SF-295 CNS/glioblastoma | 8.4 | 94970 RL non-Hodgkin's B-cell | 3.5 |
| | | lymphoma | |
| 94914 Cerebellum | 74.7 | 94972 JM1 pre-B-cell | 3.1 |
| | | lymphoma/leukemia | |
| 96777 Cerebellum | 62.4 | 94973 Jurkat T cell leukemia | 11.6 |
| 94916 NCI-H292 Mucoepidermoid | 45.7 | 94974 TF-1 Erythroleukemia | 13.4 |
| lung carcinoma | | | |
| 94917 DMS-114 Small cell lung | 10.9 | 94975 HUT 78 T-cell lymphoma | 10.0 |
| cancer | | | |
| 94918 DMS-79 Small cell lung | 100.0 | 94977 U937 Histiocytic lymphoma | 18.2 |
| cancer/neuroendocrine | | | |
| 94919 NCI-H146 Small cell lung | 30.6 | 94980 KU-812 Myelogenous | 6.0 |
| cancer/neuroendocrine | | leukemia | |
| 94920 NCI-H526 Small cell lung | 57.4 | 769-P-Clear cell renal carcinoma | 11.3 |
| cancer/neuroendocrine | | | |
| 94921 NCI-N417 Small cell lung | 15.2 | 94983 Caki-2 Clear cell renal | 10.4 |
| cancer/neuroendocrine | | carcinoma | |
| 94923 NCI-H82 Small cell lung | 36.9 | 94984 SW 839 Clear cell renal | 2.2 |
| cancer/neuroendocrine | | carcinoma | |
| 94924 NCI-H157 Squamous cell lung | 51.1 | 94986 G401 Wilms' tumor | 6.2 |
| cancer (metastasis) | | | |
| 94925 NCI-H1155 Large cell lung | 26.6 | 94987 Hs766T Pancreatic carcinoma | 42.9 |
| cancer/neuroendocrine | | (LN metastasis) | |
| 94926 NCI-H1299 Large cell lung | 44.4 | 94988 CAPAN-1 Pancreatic | |
| cancer/neuroendocrine | | adenocarcinoma (liver metastasis) | 5.0 |
| 94927 NCI-H727 Lung carcinoid | 47.0 | 94989 SU86.86 Pancreatic carcinoma | 28.1 |
| | | (liver metastasis) | |
| 94928 NCI-UMC-11 Lung carcinoid | 60.3 | 94990 BxPC-3 Pancreatic | 6.3 |
| | | adenocarcinoma | |
| 94929 LX-1 Small cell lung cancer | 23.5 | 94991 HPAC Pancreatic | 7.4 |
| | | adenocarcinoma | |
| 94930 Colo-205 Colon cancer | 29.5 | 94992 MIA PaCa-2 Pancreatic | 3.6 |
| | | carcinoma | |
| 94931 KM12 Colon cancer | 24.0 | 94993 CFPAC-1 Pancreatic ductal | 40.9 |
| | | adenocarcinoma | |
| 94932 KM20L2 Colon cancer | 8.4 | 94994 PANC-1 Pancreatic epithelioid | 20.9 |
| | | ductal carcinoma | |
| 94933 NCI-H716 Colon cancer | 23.3 | 94996 T24 Bladder carcinma | 13.1 |
| | | (transitional cell | |
| 94935 SW-48 Colon adenocarcinoma | 27.0 | 5637- Bladder carcinoma | 11.0 |
| 94936 SW1116 Colon | 10.0 | 94998 HT-1197 Bladder carcinoma | 9.2 |
| adenocarcinoma | | | |
| 94937 LS 174T Colon | 9.9 | 94999 UM-UC-3 Bladder carcinma | 7.2 |
| adenocarcinoma | | (transitional cell) | |
| 94938 SW-948 Colon | 1.1 | 95000 A204 Rhabdomyosarcoma | 7.0 |
| adenocarcinoma | | | |
| 94939 SW-480 Colon | 8.8 | 95001 HT-1080 Fibrosarcoma | 16.6 |
| adenocarcinoma | | | |
| 94940 NCI-SNU-5 Gastric carcinoma | 7.2 | 95002 MG-63 Osteosarcoma (bone) | 16.7 |
| KATO III- Gastric carcinoma | 32.8 | 95003 SK-LMS-1 Leiomyosarcoma | 26.4 |
| | | (vulva) | |
| 94943 NCI-SNU-16 Gastric | 13.6 | 95004 SJRH30 Rhabdomyosarcoma | 16.8 |
| carcinoma | | (met to bone marrow) | |
| 94944 NCI-SNU-1 Gastric carcinoma | 16.0 | 95005 A431 Epidermoid carcinoma | 9.8 |
| 94946 RF-1 Gastric adenocarcinoma | 2.1 | 95007 WM266-4 Melanoma | 12.0 |
| 94947 RF-48 Gastric adenocarcinoma | 4.0 | DU 145- Prostate carcinoma (brain | 0.1 |
| | | (metastasis) | |
| 96778 MKN-45 Gastric carcinoma | 28.7 | 95012 MDA-MB-468 Breast | 14.7 |
| | | adenocarcinoma | |
| 94949 NCI-N87 Gastric carcinoma | 13.3 | SCC-4- Squamous cell carcinoma of | 0.9 |
| | | tongue | |
| 94951 OVCAR-5 Ovarian carcinoma | 2.6 | SCC-9- Squamous cell carcinoma of | 0.3 |
| | | tongue | |
| 94952 RL95-2 Uterine carcinoma | 7.2 | SCC-15- Squamous cell carcinoma of | 0.5 |
| | | tongue | |
| 94953 HelaS3 Cervical | 13.4 | 95017 CAL 27 Squamous cell | 21.2 |
| adenocarcinoma | | carcinoma of tongue | |

TABLE BEE
Panel 4D
Column A - Rel. Exp.(%) Ag2075, Run 152787491

| Tissue Name | A | Tissue Name | A |
|---|---|---|---|
| Secondary Th1 act | 46.3 | HUVEC IL-1beta | 25.0 |
| Secondary Th2 act | 39.2 | HUVEC IFN gamma | 33.0 |
| Secondary Tr1 act | 31.9 | HUVEC TNF alpha + IFN gamma | 13.1 |
| Secondary Th1 rest | 11.7 | HUVEC TNF alpha + IL4 | 20.7 |
| Secondary Th2 rest | 13.9 | HUVEC IL-11 | 20.0 |
| Secondary Tr1 rest | 21.6 | Lung Microvascular EC none | 22.7 |
| Primary Th1 act | 30.1 | Lung Microvascular EC TNFalpha + IL- | 15.0 |
| | | 1beta | |
| Primary Th2 act | 39.2 | Microvascular Dermal EC none | 26.8 |
| Primary Tr1 act | 54.7 | Microsvasular Dermal EC TNFalpha + IL- | 16.6 |
| | | 1beta | |
| Primary Th1 rest | 84.1 | Bronchial epithelium TNFalpha + IL1beta | 4.9 |
| Primary Th2 rest | 48.6 | Small airway epithelium none | 19.9 |
| Primary Tr1 rest | 39.0 | Small airway epithelium TNFalpha + IL- | 72.7 |
| | | 1beta | |
| CD45RA CD4 lymphocyte act | 21.8 | Coronery artery SMC rest | 27.9 |
| CD45RO CD4 lymphocyte act | 33.0 | Coronery artery SMC TNFalpha + IL-1beta | 19.6 |
| CD8 lymphocyte act | 25.5 | Astrocytes rest | 26.4 |
| Secondary CD8 lymphocyte rest | 21.5 | Astrocytes TNFalpha + IL-1beta | 13.2 |
| Secondary CD8 lymphocyte act | 29.3 | KU-812 (Basophil) rest | 6.3 |
| CD4 lymphocyte none | 12.7 | KU-812 (Basophil) PMA/ionomycin | 16.0 |
| 2ry Th1/Th2/Tr1 anti-CD95 | 25.5 | CCD1106 (Keratinocytes) none | 46.0 |
| CH11 | | | |
| LAK cells rest | 26.1 | CCD1106 (Keratinocytes) TNFalpha + IL- | 4.3 |
| | | 1beta | |
| LAK cells IL-2 | 30.6 | Liver cirrhosis | 2.7 |
| LAK cells IL-2 + IL-12 | 18.2 | Lupus kidney | 3.0 |
| LAK cells IL-2 + IFN gamma | 31.0 | NCI-H292 none | 76.8 |
| LAX cells IL-2 + IL-18 | 31.2 | NCI-H292 IL-4 | 94.6 |
| LAK cells PMA/ionomycin | 9.5 | NCI-H292 IL-9 | 97.3 |
| NK Cells IL-2 rest | 37.4 | NCI-H292 IL-13 | 59.5 |
| Two Way MLR 3 day | 24.0 | NCI-H292 IFN gamma | 51.8 |
| Two Way MLR 5 day | 23.0 | HPAEC none | 23.3 |
| Two Way MLR 7 day | 19.6 | HPAEC TNF alpha + IL-1 beta | 15.8 |
| PBMC rest | 11.4 | Lung fibroblast none | 18.4 |
| PBMC PWM | 72.2 | Lung fibroblast TNF alpha + IL-1 beta | 12.0 |
| PBMC PHA-L | 33.9 | Lung fibroblast IL-4 | 35.8 |
| Ramos (B cell) none | 19.6 | Lung fibroblast IL-9 | 25.5 |
| Ramos (B cell) ionomycin | 100.0 | Lung fibroblast IL-13 | 18.7 |
| B lymphocytes PWM | 81.8 | Lung fibroblast IFN gamma | 38.4 |
| B lymphocytes CD40L and IL-4 | 42.3 | Dermal fibroblast CCD1070 rest | 48.0 |
| EOL-1 dbcAMP | 23.7 | Dermal fibroblast CCD1070 TNF alpha | 83.5 |
| EOL-1 dbcAMP | 13.5 | Dermal fibroblast CCD1070 IL-1 beta | 13.6 |
| PMA/ionomycin | | | |
| Dendritic cells none | 20.9 | Dermal fibroblast IFN gamma | 13.1 |
| Dendritic cells LPS | 11.5 | Dermal fibroblast IL-4 | 36.6 |
| Dendritic cells anti-CD40 | 23.2 | IBD Colitis 2 | 1.8 |
| Monocytes rest | 19.2 | IBD Crohn's | 2.4 |
| Monocytes LPS | 6.5 | Colon | 26.8 |
| Macrophages rest | 36.1 | Lung | 21.3 |
| Macrophages LPS | 13.3 | Thymus | 41.5 |
| HUVEC none | 37.6 | Kidney | 24.3 |
| HUVEC starved | 58.6 | | |

Panel 1.3D Summary: Ag2075 Highest expression of the CG56870-01 and CG56870-06 genes was detected in the cerebral cortex (CT=24.2). Thus expression of this gene is useful in distinguishing this sample from other samples in the panel. Significant expression of this gene is observed throughout the CNS, including in amygdala, substantia nigra, thalamus, cerebellum, cerebral cortex, and spinal cord. The CG56870-01 and CG56870-06 genes encode an Ndr3 homolog which is a putative member of Ndr family. This family consists of proteins from different gene families: Ndr1/RTP/Drg1/NDRG1, Ndr2, and Ndr3 (PFAM: IPR004142). NDRG1 is a cytoplasmic protein involved in stress responses, hormone responses, cell growth, and differentiation. Mutation of this gene was reported to be causative for hereditary motor and sensory neuropathy-Lom. Recently, NDRG4, another memember of Ndr family, was shown to be expressed in neurons of the brain and spinal cord. Its expression was markedly decreased in the brain of Alzheimer's disease patient (Zhou R H, Kokame K, Tsukamoto Y, Yutani C, Kato H, Miyata T. (2001) Characterization of the human NDRG gene family: a newly identified member, NDRG4, is specifically expressed in brain and heart. Genomics 73(1):86-97). Therapeutic modulation of this gene, expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia and depression.

This gene also showed moderate levels of expression in adipose, adrenal, thyroid, liver, heart, thyroid and skeletal muscle. Therapeutic modulation of this gene, expressed protein and/or use of antibodies or small molecule drugs targeting the gene or gene product are useful in the treatment of metabolic and endocrine disease, including Types 1 and 2 diabetes and obesity.

In addition, there was significant expression in other samples derived from breast cancer cell lines, lung cancer cell lines, renal cancer cell lines and colon cancer cell lines. Therapeutic modulation of this gene, expressed protein and/or use of antibodies or small molecule drugs targeting the gene or gene product are useful in the treatment of breast, lung, renal or colon cancer.

Panel 2.2 Summary: Ag2075 Highest expression of the CG56870-01 and CG56870-06 genes was detected in breast cancer sample (CT=29.89). Expression of this gene is useful as a marker for breast cancer. There was significant expression in other samples derived from breast cancers, kidney cancers and colon cancers. Therapeutic modulation of this gene, expressed protein and/or use of antibodies or small molecule drugs targeting the gene or gene product are useful in the treatment of breast, kidney or colon cancer.

Panel 3D Summary: Ag2075 The expression of this gene was highest in a sample derived from a lung cancer cell line (DMS-79)(CT=26.4). There was significant expression in other samples derived from pancreatic cancer cell lines, lung cancer cell lines, brain cancer cell lines and cervical cancer cell lines. Expression of this gene is useful as a marker for pancreatic, lung, brain and cervical cancers. Therapeutic modulation of this gene, expressed protein and/or use of antibodies or small molecule drugs targeting the gene or gene product are useful in the treatment of pancreatic, lung, brain or cervical cancer.

Panel 4D Summary: Ag2075 Expression of the CG56870-01 and CG56870-06 genes was ubiquitous througout this panel, with highest expression in samples derived from ionomycin treated Ramos (B cell) cells (CT=26.1). Expression was also detected in PWM treated PBMC cells and PWM treated B lymphocytes. Therapeutic modulation of these gene, expressed proteins and/or use of antibodies or small molecule drugs targeting the genes or gene products are useful in the treatment of autoimmune and inflammatory diseases in which B cells play a part in the initiation or progression of the disease process, such as systemic lupus erythematosus, Crohn's disease, ulcerative colitis, multiple sclerosis, chronic obstructive pulmonary disease, asthma, emphysema, rheumatoid arthritis, or psoriasis.

BF. CG57109-01 and CG57109-05: Doublecortin/CAMkinase.

Expression of genes CG57109-01 and CG57109-05 was assessed using the primer-probe sets Ag1137, Ag1150, Ag1860, Ag3112 and Ag4281, described in Tables BFA, BFB, BFC, BFD and BFE. Results of the RTQ-PCR runs are shown in Tables BFF, BFG, BFH, BFI, BFJ, BFK, BFL and BFM. CG57109-05 represents a full-length physical clone of the CG57109-01 gene.

TABLE BFA
Probe Name Ag1137

| | | | Start | SEQ ID |
|---|---|---|---|---|
| Primers | Sequences | Length | Position | No |
| Forward | 5′-gacatggtggacagtgagatct-3′ | 22 | 1338 | 1377 |
| Probe | TET-5′-cctctctcaccccaacatcgtgaaat-3′- | 26 | 1373 | 1378 |
| | TAMRA | | | |
| Reverse | 5′-tctgtttcgtagacttcatgca-3′ | 22 | 1399 | 1379 |

TABLE BFB
Probe Name Ag1150

| | | | Start | SEQ ID |
|---|---|---|---|---|
| Primers | Sequences | Length | Position | No |
| Forward | 5′-gaaattggctgattttggactt-3′ | 22 | 1634 | 1380 |
| Probe | TET-5′-cctatatttactgtgtgtgggacccca- | 27 | 1674 | 1381 |
| | 3′-TAMRA | | | |
| Reverse | 5′-agaatttcgggagctacgtaag-3′ | 22 | 1702 | 1382 |

TABLE BFC
Probe Name Ag1860

| | | | Start | SEQ ID |
|---|---|---|---|---|
| Primers | Sequences | Length | Position | No |
| Forward | 5′-gacatggtggacagtgagatct-3′ | 22 | 1338 | 1383 |
| Probe | TET-5′-cctctctcaccccaacatcgtgaaat-3′- | 26 | 1373 | 1384 |
| | TAMRA | | | |
| Reverse | 5′-tctgtttcgtagacttcatgca-3′ | 22 | 1399 | 1385 |

TABLE BFD
Probe Name Ag3112

| | | | Start | SEQ ID |
|---|---|---|---|---|
| Primers | Sequences | Length | Position | No |
| Forward | 5′-gaaattggctgattttggactt-3′ | 22 | 1634 | 1386 |
| Probe | TET-5′-cctatatttactgtgtgtgggacccca- | 27 | 1674 | 1387 |
| | 3′-TAMRA | | | |
| Reverse | 5′-agaatttcgggagctacgtaag-3′ | 22 | 1702 | 1388 |

TABLE BFE
Probe Name Ag4281

| | | | Start | SEQ ID |
|---|---|---|---|---|
| Primers | Sequences | Length | Position | No |
| Forward | 5′-gacatggtggacagtgagatct-3′ | 22 | 1338 | 1389 |
| Probe | TET-5′-atccagagcctctctcaccccaacat-3′- | 26 | 1365 | 1390 |
| | TAMRA | | | |
| Reverse | 5′-tcgtagacttcatgcaatttca-3′ | 22 | 1393 | 1391 |

TABLE BFF
AI.05 chondrosarcoma
Column A - Rel. Exp.(%) Ag1860, Run 306913837

| Tissue Name | A | Tissue Name | A |
|---|---|---|---|
| 138353 PMA (18 hrs) | 1.6 | 138346 IL-1beta + Oncostatin M | 21.2 |
| | | (6 hrs) | |
| 138352 IL-1beta + Oncostatin M | 2.6 | 138345 IL-1beta + TNFa (6 hrs) | 95.9 |
| (18 hrs) | | | |
| 138351 IL-1beta + TNFa (18 hrs) | 100.0 | 138344 IL-1beta (6 hrs) | 29.9 |
| 138350 IL-1beta (18 hrs) | 6.1 | 138348 Untreated-complete medium | 0.0 |
| | | (6 hrs) | |
| 138354 Untreated-complete medium | 1.3 | 138349 Untreated-serum starved | 0.0 |
| (18 hrs) | | (6 hrs) | |
| 138347 PMA (6 hrs) | 7.7 | | |

TABLE BFG
AI_{—comprehensive} panel_v1.0
Column A - Rel. Exp.(%) Ag1860, Run 225404259

| Tissue Name | A | Tissue Name | A |
|---|---|---|---|
| 110967 COPD-F | 3.7 | 112427 Match Control Psoriasis-F | 7.9 |
| 110980 COPD-F | 0.0 | 112418 Psoriasis-M | 13.1 |
| 110968 COPD-M | 20.0 | 112723 Match Control Psoriasis-M | 17.9 |
| 110977 COPD-M | 3.0 | 112419 Psoriasis-M | 26.8 |
| 110989 Emphysema-F | 26.2 | 112424 Match Control Psoriasis-M | 19.5 |
| 110992 Emphysema-F | 4.2 | 112420 Psoriasis-M | 36.9 |
| 110993 Emphysema-F | 11.0 | 112425 Match Control Psoriasis-M | 23.3 |
| 110994 Emphysema-F | 19.8 | 104689 (ME) OA Bone-Backus | 13.0 |
| 110995 Emphysema-F | 8.5 | 104690 (ME) Adj “Normal” Bone- | 3.9 |
| | | Backus | |
| 110996 Emphysema-F | 0.0 | 104691 (ME) OA Synovium-Backus | 29.5 |
| 110997 Asthma-M | 17.2 | 104692 (BA) OA Cartilage-Backus | 0.0 |
| 111001 Asthma-F | 38.4 | 104694 (BA) OA Bone-Backus | 17.3 |
| 111002 Asthma-F | 27.9 | 104695 (BA) Adj “Normal” Bone- | 0.0 |
| | | Backus | |
| 111003 Atopic Asthma-F | 50.7 | 104696 (BA) OA Synovium-Backus | 93.3 |
| 111004 Atopic Asthma-F | 68.3 | 104700 (SS) OA Bone-Backus | 11.7 |
| 111005 Atopic Asthma-F | 56.6 | 104701 (SS) Adj “Normal” Bone-Backus | 15.3 |
| 111006 Atopic Asthma-F | 17.9 | 104702 (SS) OA Synovium-Backus | 100.0 |
| 111417 Allergy-M | 31.0 | 117093 OA Cartilage Rep7 | 19.1 |
| 112347 Allergy-M | 8.3 | 112672 OA Bone5 | 6.8 |
| 112349 Normal Lung-F | 2.5 | 112673 OA Synovium5 | 4.8 |
| 112357 Normal Lung-F | 4.4 | 112674 OA Synovial Fluid cells5 | 13.9 |
| 112354 Normal Lung-M | 0.0 | 117100 OA Cartilage Rep14 | 4.5 |
| 112374 Crohns-F | 27.5 | 112756 OA Bone9 | 63.7 |
| 112389 Match Control Crohns-F | 9.0 | 112757 OA Synovium9 | 17.7 |
| 112375 Crohns-F | 36.6 | 112758 OA Synovial Fluid Cells9 | 11.7 |
| 112732 Match Control Crohns-F | 0.0 | 117125 RA Cartilage Rep2 | 27.5 |
| 112725 Crohns-M | 4.4 | 113492 Bone2 RA | 20.0 |
| 112387 Match Control Crohns-M | 17.1 | 113493 Synovium2 RA | 6.8 |
| 112378 Crohns-M | 4.2 | 113494 Syn Fluid Cells RA | 8.8 |
| 112390 Match Control Crohns-M | 49.3 | 113499 Cartilage4 RA | 4.7 |
| 112726 Crohns-M | 30.1 | 113500 Bone4 RA | 8.8 |
| 112731 Match Control Crohns-M | 0.0 | 113501 Synovium4 RA | 3.5 |
| 112380 Ulcer Col-F | 37.9 | 113502 Syn Fluid Cells4 RA | 4.0 |
| 112734 Match Control Ulcer Col-F | 17.9 | 113495 Cartilage3 RA | 10.4 |
| 112384 Ulcer Col-F | 29.7 | 113496 Bone3 RA | 4.2 |
| 112737 Match Control Ulcer Col-F | 9.0 | 113497 Synovium3 RA | 0.0 |
| 112386 Ulcer Col-F | 5.0 | 113498 Syn Fluid Cells3 RA | 7.9 |
| 112738 Match Control Ulcer Col-F | 9.8 | 117106 Normal Cartilage Rep20 | 0.0 |
| 112381 Ulcer Col-M | 0.0 | 113663 Bone3 Normal | 0.0 |
| 112735 Match Control Ulcer Col-M | 28.7 | 113664 Synovium3 Normal | 0.0 |
| 112382 Ulcer Col-M | 10.0 | 113665 Syn Fluid Cells3 Normal | 0.0 |
| 112394 Match Control Ulcer Col-M | 0.0 | 117107 Normal Cartilage Rep22 | 0.0 |
| 112383 Ulcer Col-M | 35.4 | 113667 Bone4 Normal | 13.3 |
| 112736 Match Control Ulcer Col-M | 6.3 | 113668 Synovium4 Normal | 0.0 |
| 112423 Psoriasis-F | 21.5 | 113669 Syn Fluid Cells4 Normal | 9.4 |

TABLE BFH
General_screening_panel_v1.4
Column A - Rel. Exp.(%) Ag4281, Run 222183233

| Tissue Name | A | Tissue Name | A |
|---|---|---|---|
| Adipose | 0.5 | Renal ca.TK-10 | 0.0 |
| Melanoma* Hs688(A).T | 0.0 | Bladder | 5.4 |
| Melanoma* Hs688(B).T | 0.0 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.2 |
| Squamous cell carcinoma SCC-4 | 0.0 | Colon ca.* (SW480 met) SW620 | 0.0 |
| Testis Pool | 34.2 | Colon ca. HT29 | 0.0 |
| Prostate ca.* (bone met) PC-3 | 0.0 | Colon ca. HCT-116 | 0.0 |
| Prostate Pool | 0.9 | Colon ca. CaCo-2 | 2.6 |
| Placenta | 2.0 | Colon cancer tissue | 5.6 |
| Uterus Pool | 0.8 | Colon ca. SW1116 | 0.0 |
| Ovarian ca. OVCAR-3 | 0.7 | Colon ca. Colo-205 | 0.0 |
| Ovarian ca. SK-OV-3 | 0.6 | Colon ca. SW-48 | 0.0 |
| Ovarian ca. OVCAR-4 | 0.0 | Colon Pool | 4.0 |
| Ovarian ca. OVCAR-5 | 0.0 | Small Intestine Pool | 0.5 |
| Ovarian ca. IGROV-1 | 0.0 | Stomach Pool | 0.4 |
| Ovarian ca. OVCAR-8 | 0.0 | Bone Marrow Pool | 1.1 |
| Ovary | 0.6 | Fetal Heart | 4.5 |
| Breast ca. MCF-7 | 0.0 | Heart Pool | 2.7 |
| Breast ca. MDA-MB-231 | 0.0 | Lymph Node Pool | 5.9 |
| Breast ca. BT 549 | 0.0 | Fetal Skeletal Muscle | 22.1 |
| Breast ca. T47D | 1.0 | Skeletal Muscle Pool | 9.3 |
| Breast ca. MDA-N | 0.0 | Spleen Pool | 2.4 |
| Breast Pool | 3.1 | Thymus Pool | 3.1 |
| Trachea | 1.7 | CNS cancer (glio/astro) U87-MG | 0.0 |
| Lung | 0.0 | CNS cancer (glio/astro) U-118-MG | 0.0 |
| Fetal Lung | 14.4 | CNS cancer (neuro; met) SK-N-AS | 0.8 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.9 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 25.5 |
| Lung ca. NCI-H526 | 0.0 | Brain (cerebellum) | 5.4 |
| Lung ca. NCI-H23 | 2.1 | Brain (fetal) | 100.0 |
| Lung ca. NCI-H460 | 0.0 | Brain (Hippocampus) Pool | 59.0 |
| Lung ca. HOP-62 | 0.0 | Cerebral Cortex Pool | 14.1 |
| Lung ca. NCI-H522 | 0.7 | Brain (Substantia nigra) Pool | 15.1 |
| Liver | 0.3 | Brain (Thalamus) Pool | 28.9 |
| Fetal Liver | 1.0 | Brain (whole) | 33.9 |
| Liver ca. HepG2 | 0.0 | Spinal Cord Pool | 25.2 |
| Kidney Pool | 4.2 | Adrenal Gland | 2.3 |
| Fetal Kidney | 4.6 | Pituitary gland Pool | 36.6 |
| Renal ca. 786-0 | 0.0 | Salivary Gland | 0.4 |
| Renal ca. A498 | 0.0 | Thyroid (female) | 0.4 |
| Renal ca. ACHN | 0.0 | Pancreatic ca. CAPAN2 | 0.0 |
| Renal ca. UO-31 | 0.0 | Pancreas Pool | 2.1 |

TABLE BFI
PGI1.0
Column A - Rel. Exp.(%) Ag3112, Run 395549582

| Tissue Name | A | Tissue Name | A |
|---|---|---|---|
| 162191 Normal Lung 1 (IBS) | 0.5 | 162185 Emphysema Lung 12 (Ardais) | 5.4 |
| 160468 MD lung | 6.8 | 162184 Emphysema Lung 13 (Ardais) | 6.5 |
| 156629 MD Lung 13 | 2.3 | 162183 Emphysema Lung 14 (Ardais) | 13.3 |
| 162570 Normal Lung 4 (Aastrand) | 2.0 | 162188 Emphysema Lung 15 (Genomic | 42.9 |
| | | Collaborative) | |
| 162571 Normal Lung 3 (Aastrand) | 0.8 | 162177 NAT UC Colon 1 (Ardais) | 15.3 |
| 162187 Fibrosis Lung 2 (Genomic | 99.3 | 162176 UC Colon 1 (Ardais) | 21.0 |
| Collaborative) | | | |
| 151281 Fibrosis lung 11(Ardais) | 24.0 | 162179 NAT UC Colon 2(Ardais) | 5.8 |
| 162186 Fibrosis Lung 1 (Genomic | 35.6 | 162178 UC Colon 2(Ardais) | 38.2 |
| Collaborative) | | | |
| 162190 Asthma Lung 4 (Genomic | 53.2 | 162181 NAT UC Colon 3(Ardais) | 3.9 |
| Collaborative) | | | |
| 160467 Asthma Lung 13 (MD) | 0.6 | 162180 UC Colon 3(Ardais) | 20.3 |
| 137027 Emphysema Lung 1 | 2.3 | 162182 NAT UC Colon 4 (Ardais) | 5.1 |
| (Ardais) | | | |
| 137028 Emphysema Lung 2 | 0.0 | 137042 UC Colon 1108 | 27.4 |
| (Ardais) | | | |
| 137040 Emphysema Lung 3 | 4.4 | 137029 UC Colon 8215 | 100.0 |
| (Ardais) | | | |
| 137041 Emphysema Lung 4 | 4.0 | 137031 UC Colon 8217 | 66.4 |
| (Ardais) | | | |
| 137043 Emphysema Lung 5 | 15.0 | 137036 UC Colon 1137 | 43.5 |
| (Ardais) | | | |
| 142817 Emphysema Lung 6 | 7.6 | 137038 UC Colon 1491 | 62.4 |
| (Ardais) | | | |
| 142818 Emphysema Lung 7 | 5.9 | 137039 UC Colon 1546 | 78.5 |
| (Ardais) | | | |
| 142819 Emphysema Lung 8 | 8.2 | 162593 Crohn's 47751 (NDRI) | 9.4 |
| (Ardais) | | | |
| 142820 Emphysema Lung 9 | 4.0 | 162594 NAT Crohn's 47751 (NDRI) | 3.9 |
| (Ardais) | | | |
| 142821 Emphysema Lung 10 | 6.7 | | |
| (Ardais) | | | |

TABLE BFK
Panel 3D
Column A - Rel. Exp.(%) Ag3112, Run 182114339

| Tissue Name | A | Tissue Name | A |
|---|---|---|---|
| 94905 Daoy | 0.0 | 94954 Ca Ski Cervical epidermoid | 0.0 |
| Medulloblastoma/Cerebellum | | carcinoma (metastasis | |
| 94906 TE671 | 0.0 | 94955 ES-2 Ovarian clear cell | 0.0 |
| Medulloblastom/Cerebellum | | carcinoma | |
| 94907 D283 Med | 0.0 | 94957 Ramos Stimulated with | 0.0 |
| Medulloblastoma/Cerebellum | | PMA/ionomycin 6 h | |
| 94908 PFSK-1 Primitive | 0.0 | 94958 Ramos Stimulated with | 0.0 |
| Neuroectodermal/Cerebellum | | PMA/ionomycin 14 h | |
| 94909 XF-498 CNS | 0.0 | 94962 MEG-01 Chronic myelogenous | 0.0 |
| | | leukemia (megokaryoblast) | |
| 94910 SNB-78 CNS/glioma | 0.0 | 94963 Raji Burkitt's lymphoma | 0.0 |
| 94911 SF-268 CNS/glioblastoma | 0.0 | 94964 Daudi Burkitt's lymphoma | 0.0 |
| 94912 T98G Glioblastoma | 0.0 | 94965 U266 B-cell | 0.0 |
| | | plasmacytoma/myeloma | |
| 96776 SK-N-SH Neuroblastoma | 0.0 | 94968 CA46 Burkitt's lymphoma | 0.0 |
| (metastasis) | | | |
| 94913 SF-295 CNS/glioblastoma | 0.0 | 94970 RL non-Hodgkin's B-cell | 0.0 |
| | | lymphoma | |
| 94914 Cerebellum | 6.5 | 94972 JM1 pre-B-cell | 0.0 |
| | | lymphoma/leukemia | |
| 96777 Cerebellum | 2.4 | 94973 Jurkat T cell leukemia | 7.3 |
| 94916 NCI-H292 Mucoepidermoid | 0.0 | 94974 TF-1 Erythroleukemia | 0.0 |
| lung carcinoma | | | |
| 94917 DMS-114 Small cell lung | 0.0 | 94975 HUT 78 T-cell lymphoma | 0.0 |
| cancer | | | |
| 94918 DMS-79 Small cell lung | 100.0 | 94977 U937 Histiocytic lymphoma | 0.0 |
| cancer/neuroendocrine | | | |
| 94919 NCI-H146 Small cell lung | 0.0 | 94980 KU-812 Myelogenous | 0.0 |
| cancer/neuroendocrine | | leukemia | |
| 94920 NCI-H526 Small cell lung | 0.0 | 769-P- Clear cell renal carcinoma | 0.0 |
| cancer/neuroendocrine | | | |
| 94921 NCI-N417 Small cell lung | 0.0 | 94983 Caki-2 Clear cell renal | 0.0 |
| cancer/neuroendocrine | | carcinoma | |
| 94923 NCI-H82 Small cell lung | 0.0 | 94984 SW 839 Clear cell renal | 0.0 |
| cancer/neuroendocrine | | carcinoma | |
| 94924 NCI-H157 Squamous cell lung | 0.0 | 94986 G401 Wilims' tumor | 0.0 |
| cancer (metastasis) | | | |
| 94925 NCI-H1155 Large cell lung | 3.6 | 94987 Hs766T Pancreatic carcinoma | 0.0 |
| cancer/neuroendocrine | | (LN metastasis) | |
| 94926 NCI-H1299 Large cell lung | 0.0 | 94988 CAPAN-1 Pancreatic | 0.0 |
| cancer/neuroendocrine | | adenocarcinoma (liver metastasis) | |
| 94927 NCI-H727 Lung carcinoid | 0.0 | 94989 SU86.86 Pancreatic carcinoma | 0.0 |
| | | (liver metastasis) | |
| 94928 NCI-UMC-11 Lung carcinoid | 0.0 | 94990 BxPC-3 Pancreatic | 0.0 |
| | | adenocarcinoma | |
| 94929 LX-1 Small cell lung cancer | 0.0 | 94991 HPAC Pancreatic | 0.0 |
| | | adenocarcinoma | |
| 94930 Colo-205 Colon cancer | 0.0 | 94992 MIA PaCa-2 Pancreatic | 0.0 |
| | | carcinoma | |
| 94931 KM12 Colon cancer | 0.0 | 94993 CFPAC-1 Pancreatic ductal | 0.0 |
| | | adenocarcinoma | |
| 94932 KM20L2 Colon cancer | 0.0 | 94994 PANC-1 Pancreatic epithelioid | 0.0 |
| | | ductal carcinoma | |
| 94933 NCI-H716 Colon cancer | 0.0 | 94996 T24 Bladder carcinma | 0.0 |
| | | (transitional cell | |
| 94935 SW-48 Colon adenocarcinoma | 0.0 | 5637- Bladder carcinoma | 0.0 |
| 94936 SW1116 Colon adenocarcinoma | 0.0 | 94998 HT-1197 Bladder carcinoma | 0.0 |
| 94937 LS 174T Colon | 0.0 | 94999 UM-UC-3 Bladder carcinma | 0.0 |
| adenocarcinoma | | (transitional cell) | |
| 94938 SW-948 Colon adenocarcinoma | 0.0 | 95000 A204 Rhabdomyosarcoma | 0.0 |
| 94939 SW-480 Colon adenocarcinoma | 0.0 | 95001 HT-1080 Fibrosarcoma | 0.0 |
| 94940 NCI-SNU-5 Gastric carcinoma | 0.0 | 95002 MG-63 Osteosarcoma (bone) | 0.0 |
| KATO III- Gastnc carcinoma | 0.0 | 95003 SK-LMS-1 Leiomyosarcoma | 0.0 |
| | | (vulva) | |
| 94943 NCI-SNU-16 Gastric carcinoma | 0.0 | 95004 SJRH30 Rhabdomyosarcoma | 0.0 |
| | | (met to bone marrow) | |
| 94944 NCI-SNU-1 Gastric carcinoma | 0.0 | 95005 A431 Epidermoid carcinoma | 0.0 |
| 94946 RE-1 Gastric adenocarcinoma | 0.0 | 95007 WM266-4 Melanoma | 0.0 |
| 94947 RF-48 Gastric adenocarcinoma | 0.0 | DU 145- Prostate carcinoma (brain | 0.0 |
| | | metastasis) | |
| 96778 MKN-45 Gastric carcinoma | 0.0 | 95012 MDA-MB-468 Breast | 0.0 |
| | | adenocarcinoma | |
| 94949 NCI-N87 Gastric carcinoma | 0.0 | SCC-4- Squamous cell carcinoma of | 0.0 |
| | | tongue | |
| 94951 OVCAR-5 Ovarian carcinoma | 0.0 | SCC-9- Squamous cell carcinoma of | 0.0 |
| | | tongue | |
| 94952 RL95-2 Uterine carcinoma | 0.0 | SCC-15- Squamous cell carcinoma of | 0.0 |
| | | tongue | |
| 94953 Hela53 Cervical | 0.0 | 95017 CAL 27 Squamous cell | 0.0 |
| adenocarcinoma | | carcinoma of tongue | |

TABLE BFM
Panel 4D
Column A - Rel. Exp. (%) Ag1860, Run 165828919
Column B - Rel. Exp. (%) Ag3112, Run 164526081

| Tissue Name | A | B | Tissue Name | A | B |
|---|---|---|---|---|---|
| Secondary Th1 act | 0.0 | 0.0 | HUVEC IL-1 beta | 6.5 | 2.2 |
| Secondary Th2 act | 0.0 | 0.0 | HUVEC IFN gamma | 0.0 | 0.0 |
| Secondary Tr1 act | 0.8 | 0.0 | HUVEC TNF alpha + IFN gamma | 0.6 | 0.0 |
| Secondary Th1 rest | 0.0 | 1.7 | HUVEC TNF alpha + IL4 | 0.6 | 0.0 |
| Secondary Th2 rest | 0.0 | 0.0 | HUVEC IL-11 | 0.0 | 0.0 |
| Secondary Tr1 rest | 0.6 | 0.0 | Lung Microvascular EC none | 0.0 | 0.0 |
| Primary Th1 act | 0.0 | 0.0 | Lung Microvascular EC TNF alpha + | 9.7 | 9.7 |
| | | | IL-1 beta | | |
| Primary Th2 act | 0.0 | 0.0 | Microvascular Dermal EC none | 0.0 | 0.0 |
| Primary Tr1 act | 0.7 | 0.0 | Microsvasular Dermal EC TNF alpha + | 1.4 | 5.9 |
| | | | IL-1 beta | | |
| Primary Th1 rest | 0.7 | 0.0 | Bronchial epithelium TNF alpha + | 0.6 | 0.0 |
| | | | IL1 beta | | |
| Primary Th2 rest | 0.6 | 0.0 | Small airway epithelium none | 0.0 | 0.0 |
| Primary Tr1 rest | 0.0 | 0.0 | Small airway epithelium TNF alpha + | 0.0 | 0.0 |
| | | | IL-1 beta | | |
| CD45RA CD4 lymphocyte | 6.7 | 2.9 | Coronery artery SMC rest | 9.7 | 10.4 |
| act | | | | | |
| CD45RO CD4 lymphocyte | 0.0 | 0.0 | Coronery artery SMC TNF alpha + | 23.7 | 32.8 |
| act | | | IL-1 beta | | |
| CD8 lymphocyte act | 1.4 | 0.0 | Astrocytes rest | 0.0 | 0.0 |
| Secondary CD8 lymphocyte | 0.0 | 0.0 | Astrocytes TNF alpha + IL-1 beta | 11.7 | 0.0 |
| rest | | | | | |
| Secondary CD8 lymphocyte | 0.0 | 1.9 | KU-812 (Basophil) rest | 0.0 | 0.0 |
| act | | | | | |
| CD4 lymphocyte none | 0.0 | 0.0 | KU-812 (Basophil) PMA/ionomycin | 0.0 | 0.0 |
| 2ry Th1/Th2/Tr1 anti-CD95 | 0.0 | 2.0 | CCD1106 (Keratinocytes) none | 0.0 | 0.0 |
| CH11 | | | | | |
| LAK cells rest | 0.9 | 0.0 | CCD1106 (Keratinocytes) TNF alpha + | 0.0 | 0.0 |
| | | | IL-1 beta | | |
| LAK cells IL-2 | 2.7 | 0.0 | Liver cirrhosis | 20.0 | 6.4 |
| LAK cells IL-2 + IL-12 | 0.0 | 0.0 | Lupus kidney | 0.0 | 0.0 |
| LAK cells IL-2 + IFN | 0.4 | 0.0 | NCI-H292 none | 0.0 | 0.0 |
| gamma | | | | | |
| LAK cells IL-2 + IL-18 | 0.0 | 0.0 | NCI-H292 IL-4 | 0.0 | 0.0 |
| LAK cells PMA/ionomycin | 1.8 | 0.0 | NCI-H292 IL-9 | 0.0 | 2.6 |
| NK Cells IL-2 rest | 1.7 | 0.0 | NCI-H292 IL-13 | 0.0 | 1.7 |
| Two Way MLR 3 day | 1.6 | 0.0 | NCI-H292 IFN gamma | 0.0 | 0.0 |
| Two Way MLR 5 day | 0.0 | 0.0 | HPAEC none | 0.0 | 0.0 |
| Two Way MLR 7 day | 0.0 | 0.0 | HPAEC TNF alpha + IL-1 beta | 100.0 | 69.3 |
| PBMC rest | 0.8 | 0.0 | Lung fibroblast none | 0.0 | 0.0 |
| PBMC PWM | 1.5 | 1.2 | Lung fibroblast TNF alpha + IL-1 | 16.4 | 3.5 |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) ionomycin | 0.0 | 0.0 | Lung fibroblast IL-13 | 0.7 | 0.0 |
| B lymphocytes PWM | 0.0 | 2.5 | Lung fibroblast IFN gamma | 0.0 | 0.0 |
| B lymphocytes CD40L and | 0.0 | 1.7 | Dermal fibroblast CCD1070 rest | 2.3 | 4.1 |
| IL-4 | | | | | |
| EOL-1 dbcAMP | 0.0 | 0.0 | Dermal fibroblast CCD1070 TNF | 28.7 | 42.6 |
| | | | alpha | | |
| EOL-1 dbcAMP | 0.0 | 0.0 | Dermal fibroblast CCD1070 IL-1 | 49.7 | 100.0 |
| PMA/ionomycin | | | beta | | |
| Dendritic cells none | 4.6 | 0.0 | Dermal fibroblast IFN gamma | 0.0 | 0.9 |
| Dendritic cells LPS | 4.2 | 2.9 | Dermal fibroblast IL-4 | 0.3 | 0.0 |
| Dendritic cells anti-CD40 | 15.8 | 11.5 | IBD Colitis 2 | 1.9 | 0.0 |
| Monocytes rest | 0.0 | 0.0 | IBD Crohn's | 2.7 | 5.1 |
| Monocytes LPS | 2.3 | 0.0 | Colon | 15.6 | 3.2 |
| Macrophages rest | 5.7 | 3.4 | Lung | 23.0 | 27.7 |
| Macrophages LPS | 6.8 | 3.4 | Thymus | 0.0 | 1.7 |
| HUVEC none | 0.0 | 0.0 | Kidney | 26.8 | 27.5 |
| HUVEC starved | 0.0 | 0.0 | | | |

AI.05 chondrosarcoma Summary: Ag1860 Highest expression was detected in IL-1TNF-a treated chondrosarcoma cells (SW1353 cell lines). Expression of this gene was up-regulated upon IL-1 treatment, a potent activator of pro-inflammatory cytokines and matrix metalloproteinases, which participate in the destruction of cartilage observed in Osteoarthritis (OA). Therapeutic modulation of these genes, expressed proteins and/or use of antibodies or small molecule drugs targeting the genes or gene products are useful in the treatment of the degeneration of cartilage observed in OA.

AI_comprehensive panel_v1.0 Summary: Ag1860 Highest expression in this panel was seen in synovium from an OA patient (CT=33.7). Overall, the CG57109-01 and CG57109-05 genes were expressed in OA tissue but not in normal joint tissue and were expressed in pulmonary tissue from patients with atopic asthma but not in normal lung tissue. Therapeutic modulation of these genes, expressed proteins and/or use of antibodies or small molecule drugs targeting the genes or gene products are useful in the treatment of inflammatory diseases including OA and asthma.

General_screening_panel_v1.4 Summary: Ag4281 Highest expression of the CG57109-01 and CG57109-05 genes was detected in the fetal brain (CT=29.5). Overall, expression of this gene was highly brain-specific in this panel, with moderate levels of expression in the amygdala, hippocampus, thalamus and spinal cord and low but significant levels in the cerebral cortex and the substantia nigra. CG57109-01 and CG57109-05 encode a novel doublecortin/CAM kinase like protein. Other members of this family have been implicated in the calcium-signaling pathway that controls neuronal migration in the developing brain. In addition, CAM kinase has been shown to play a crucial role in hippocampal Long Term Potentiation (LTP) from studies in transgenic and knock-out mice, and may also play a role in memory formation in the mature nervous system as well as the developing brain. CAM kinases have also been shown to phosporylate tau, an integral component of the neurofibrillary tangles seen in Alzheimer's, in a manner which shifts tau electrophorytic motility to that seen in the AD brain. Furthermore, tau from AD brains shows aberrent phosphorylation. Therapeutic modulation of these gene, expressed protein and/or use of antibodies or small molecule drugs targeting the genes or gene products are useful in the treatment of learning and memory deficits that are a result of aging or neurodegenerative disease and also in the treatment of neurologic disorders themselves, including Alzheimer's disease.

Moderate to low levels of expression were also seen in a variety of samples from normal tissues, including testis, fetal and adult heart and skeletal muscle and fetal lung.

Expression was much higher in fetal lung (CT=32.3) when compared to expression in the adult counterpart (CT=40). Expression of this gene is useful for distinguishing between the fetal and adult source of this tissue.

PGI1.0 Summary: Ag3112 Highest expression was detected in a colon sample from an ulcerative colitis patient (CT=30.7). Strong expression was observed in a cluster of colon samples derived from ulcerative colitis patients and in fibrotic lung samples. Therapeutic modulation of this gene, expressed protein and/or use of antibodies or small molecule drugs targeting the gene or gene product are useful in the treatment of inflammatory conditions of the colon and lung.

Panel 3D Summary: Ag3112 Expression was restricted to a sample derived from a lung cancer cell line (CT=32.6). Expression of this gene or expressed protein could be used to detect the presence of lung cancer. Therapeutic modulation of these genes, expressed proteins and/or use of antibodies or small molecule drugs targeting the genes or gene products are useful in the treatment of lung cancer.

Panel 4D Summary: Ag1860 This transcript was highly expressed in activated dermal fibroblasts, endothelial cells, and astrocytes after treatment with IL-1 or TNFalpha, with highest expression in TNF alpha and IL-1 beta treated HPAECs (CT=30.9). The proteins encoded by the CG57109-01 and CG57109-05 genes have homology to protein kinase and may be involved in leukocyte extravasation from the peripheral blood into tissues (Borbiev T, Am J Physiol Lung Cell Mol Physiol 2001 May; 280(5):L983-90). Therapeutic modulation of these genes, expressed proteins and/or use of antibodies or small molecule drugs targeting the genes or gene products are useful in the treatment of inflammation due to asthma, allergy, emphysema, osteoarthritis, colitis, psoriasis, or delayed type hypersensitivity. Agonistic therapies are useful for directing leukocyte traffic into tumors or sites of infection.

Ag3112 Highest expression of the transcript was seen in IL-1 beta treated dermal fibroblasts (CT=30.4). Expression was in agreement with the profile seen with Ag1860, except no expression was seen in astrocytes.

BG. CG57399-04: Phospholipase ADRAB-B Precursor.

Expression of gene CG57399-04 was assessed using the primer-probe set Ag3952, described in Table BGA. Results of the RTQ-PCR runs are shown in Tables BGB and BGC. CG57399-04 represents a full-length physical clone of the CG57399-02 gene.

TABLE BGA
Probe Name Ag3952

| | | | Start | SEQ ID |
|---|---|---|---|---|
| Primers | Sequences | Length | Position | No |
| Forward | 5′-ctgtgtccctgtgtcctgaa-3′ | 20 | 274 | 1392 |
| Probe | TET-5′-tcaacagaacttgctaccctcatcga-3′- | 26 | 307 | 1393 |
| | TAMRA | | | |
| Reverse | 5′-gtgggtcttctcctgaaacttc-3′ | 22 | 342 | 1394 |

TABLE BGB
General_screening_panel_v1.4
Column A - Rel. Exp.(%) Ag3952, Run 213856126

| Tissue Name | A | Tissue Name | A |
|---|---|---|---|
| Adipose | 9.0 | Renal ca. TK-10 | 15.0 |
| Melanoma* Hs688(A).T | 3.0 | Bladder | 22.7 |
| Melanoma* Hs688(B).T | 3.4 | Gastric ca. (liver met.) NCI-N87 | 13.0 |
| Melanoma* M14 | 0.9 | Gastric ca. KATO III | 75.3 |
| Melanoma* LOXIMVI | 11.7 | Colon ca. SW-948 | 4.3 |
| Melanoma* SK-MEL-5 | 1.5 | Colon ca. SW480 | 97.3 |
| Squamous cell Carcinoma SCC-4 | 8.7 | Colon ca.* (SW480 met) SW620 | 4.4 |
| Testis Pool | 12.8 | Colon ca. HT29 | 0.4 |
| Prostate ca.* (bone met) PC-3 | 10.5 | Colon ca. HCT-116 | 1.2 |
| Prostate Pool | 12.9 | Colon ca. CaCo-2 | 60.7 |
| Placenta | 5.1 | Colon cancer tissue | 28.7 |
| Uterus Pool | 6.5 | Colon ca. SW1116 | 0.0 |
| Ovarian ca. OVCAR-3 | 7.3 | Colon ca. Colo-205 | 0.9 |
| Ovarian ca. SK-OV-3 | 26.4 | Colon ca. SW-48 | 26.1 |
| Ovarian ca. OVCAR-4 | 1.9 | Colon Pool | 18.8 |
| Ovarian ca. OVCAR-5 | 6.7 | Small Intestine Pool | 5.3 |
| Ovarian ca. IGROV-1 | 9.2 | Stomach Pool | 7.9 |
| Ovarian ca. OVCAR-8 | 4.2 | Bone Marrow Pool | 8.4 |
| Ovary | 10.0 | Fetal Heart | 1.2 |
| Breast ca. MCF-7 | 0.4 | Heart Pool | 5.7 |
| Breast ca. MDA-MB-231 | 92.0 | Lymph Node Pool | 32.1 |
| Breast ca. BT 549 | 5.5 | Fetal Skeletal Muscle | 1.2 |
| Breast ca. T47D | 2.5 | Skeletal Muscle Pool | 4.7 |
| Breast ca. MDA-N | 1.6 | Spleen Pool | 18.2 |
| Breast Pool | 19.6 | Thymus Pool | 19.3 |
| Trachea | 10.3 | CNS cancer (glio/astro) U87-MG | 38.2 |
| Lung | 1.2 | CNS cancer (glio/astro) U-118-MG | 12.2 |
| Fetal Lung | 8.3 | CNS cancer (neuro; met) SK-N-AS | 0.9 |
| Lung ca. NCI-N417 | 0.9 | CNS cancer (astro) SF-539 | 7.6 |
| Lung ca. LX-1 | 27.2 | CNS cancer (astro) SNB-75 | 17.1 |
| Lung ca. NCI-H146 | 10.7 | CNS cancer (glio) SNB-19 | 6.8 |
| Lung ca. SHP-77 | 47.3 | CNS cancer (glio) SF-295 | 5.7 |
| Lung ca. A549 | 5.1 | Brain (Amygdala) Pool | 7.0 |
| Lung ca. NCI-H526 | 0.0 | Brain (cerebellum) | 3.2 |
| Lung ca. NCI-H23 | 4.1 | Brain (fetal) | 19.3 |
| Lung ca. NCI-H460 | 0.5 | Brain (Hippocampus) Pool | 13.1 |
| Lung ca. HOP-62 | 2.7 | Cerebral Cortex Pool | 14.8 |
| Lung ca. NCI-H522 | 1.3 | Brain (Substantia nigra) Pool | 6.3 |
| Liver | 0.0 | Brain (Thalamus) Pool | 15.2 |
| Fetal Liver | 1.7 | Brain (whole) | 10.4 |
| Liver ca. HepG2 | 0.5 | Spinal Cord Pool | 5.3 |
| Kidney Pool | 21.2 | Adrenal Gland | 100.0 |
| Fetal Kidney | 1.6 | Pituitary gland Pool | 4.3 |
| Renal ca. 786-0 | 1.7 | Salivary Gland | 3.4 |
| Renal ca. A498 | 1.3 | Thyroid (female) | 14.5 |
| Renal ca. ACHN | 4.3 | Pancreatic ca. CAPAN2 | 1.7 |
| Renal ca. UO-31 | 17.4 | Pancreas Pool | 24.5 |

TABLE BGC
Panel 5 Islet
Column A - Rel. Exp.(%) Ag3952, Run 323591168

| Tissue Name | A | Tissue Name | A |
|---|---|---|---|
| 97457 Patient-02go adipose | 39.8 | 94709 Donor 2 AM - A adipose | 20.9 |
| 97476 Patient-07sk skeletal muscle | 0.0 | 94710 Donor 2 AM - B adipose | 9.5 |
| 97477 Patient-07ut uterus | 27.5 | 94711 Donor 2 AM - C adipose | 3.9 |
| 97478 Patient-07pl placenta | 2.8 | 94712 Donor 2 AD - A adipose | 5.7 |
| 99167 Bayer Patient 1 | 16.0 | 94713 Donor 2 AD - B adipose | 23.3 |
| 97482 Patient-08ut uterus | 6.3 | 94714 Donor 2 AD - C adipose | 5.5 |
| 97483 Patient-08pl placenta | 4.9 | 94742 Donor 3 U - A Mesenchymal | 6.9 |
| | | Stem Cells | |
| 97486 Patient-09sk skeletal muscle | 8.3 | 94743 Donor 3 U - B Mesenchymal | 0.0 |
| | | Stem Cells | |
| 97487 Patient-09ut uterus | 19.8 | 94730 Donor 3 AM - A adipose | 10.7 |
| 97488 Patient-09pl placenta | 8.0 | 94731 Donor 3 AM - B adipose | 23.5 |
| 97492 Patient-10ut uterus | 20.4 | 94732 Donor 3 AM - C adipose | 18.2 |
| 97493 Patient-10pl placenta | 18.0 | 94733 Donor 3 AD - A adipose | 9.3 |
| 97495 Patient-11go adipose | 49.7 | 94734 Donor 3 AD - B adipose | 0.0 |
| 97496 Patient-11sk skeletal muscle | 2.4 | 94735 Donor 3 AD - C adipose | 5.1 |
| 97497 Patient-11ut uterus | 26.8 | 77138 Liver HepG2untreated | 4.5 |
| 97498 Patient-11pl placenta | 12.9 | 73556 Heart Cardiac stromal cells | 0.0 |
| | | (primary) | |
| 97500 Patient-12go adipose | 31.6 | 81735 Small Intestine | 100.0 |
| 97501 Patient-12sk skeletal muscle | 11.2 | 72409 Kidney Proximal Convoluted | 7.3 |
| | | Tubule | |
| 97502 Patient-12ut uterus | 16.4 | 82685 Small intestine Duodenum | 17.2 |
| 97503 Patient-12p1 placenta | 30.1 | 90650 Adrenal Adrenocortical | |
| | | adenoma | |
| 94721 Donor 2 U - A Mesenchymal | 5.4 | 72410 Kidney HRCE | 10.2 |
| Stem Cells | | | |
| 94722 Donor 2 U - B Mesenchymal | 36.1 | 72411 Kidney HRE | 14.5 |
| Stem Cells | | | |
| 94723 Donor 2 U - C Mesenchymal | 5.2 | 73139 Uterus Uterine smooth muscle | 36.3 |
| Stem Cells | | cells | |

General_screening_panel_v1.4 Summary: Ag3952 Highest expression of this gene was seen in the adrenal gland (CT=29). Therapeutic modulation of this gene, expressed protein and/or use of antibodies or small molecule drugs targeting the gene or gene product are useful in the treatment of Addison's disease and other adrenalopathies. This gene showed significant expression in adipose, heart, skeletal muscle, pituitary, thyroid, and pancreas. Therapeutic modulation of this gene, expressed protein and/or use of antibodies or small molecule drugs targeting the gene or gene product are useful in the treatment of endocrine or metabolic disease, including Types 1 and 2 diabetes, obesity and pancreatitis.

Expression of this gene was detected in samples derived from colon, gastric, lung and breast cancers. Expression of this gene is useful for detecting the presence of these cancers. Therapeutic modulation of this gene, expressed protein and/or use of antibodies or small molecule drugs targeting the gene or gene product are useful in the treatment of colon, gastric, lung and breast cancers.

Low but significant levels of expression were seen for all regions of the CNS examined. Therapeutic modulation of this gene, expressed protein and/or use of antibodies or small molecule drugs targeting the gene or gene product are useful in the treatment of CNS disorders such as Alzheimer's disease, Parkinson's disease, stroke, epilepsy, schizophrenia and multiple sclerosis.

Panel 5 Islet Summary: Ag3952 Highest expression was detected in small intestine (CT=32.5). Low but significant expression was also detected in adipose.

BH. CG57562-02: Cation-Transporting ATPase.

Expression of gene CG57562-02 was assessed using the primer-probe sets Ag1179, Ag3287 and Ag6477, described in Tables BHA, BHB and BHC. Results of the RTQ-PCR runs are shown in Tables BHD, BHE and BHF.

TABLE BHA
Probe Name Ag1179

| | | | Start | SEQ ID |
|---|---|---|---|---|
| Primers | Sequences | Length | Position | No |
| Forward | 5′-cgctacagatgttcaagatcct-3′ | 22 | 2844 | 1395 |
| Probe | TET-5′-ctacagccagagcgtcctctacctgg-3′- | 26 | 2890 | 1396 |
| | TAMRA | | | |
| Reverse | 5′-cctggaagtcactgaacttgac-3′ | 22 | 2921 | 1397 |

TABLE BHB
Probe Name Ag3287

| | | | Start | SEQ ID |
|---|---|---|---|---|
| Primers | Sequences | Length | Position | No |
| Forward | 5′-cgctacagatgttcaagatcct-3′ | 22 | 2844 | 1398 |
| Probe | TET-5′-ctacagccagagcgtcctctacctgg-3′- | 26 | 2890 | 1399 |
| | TAMRA | | | |
| Reverse | 5′-cctggaagtcactgaacttgac-3′ | 22 | 2921 | 1400 |

TABLE BHC
Probe Name Ag6477

| | | | Start | SEQ ID |
|---|---|---|---|---|
| Primers | Sequences | Length | Position | No |
| Forward | 5′-ctctgcactccatggccc-3′ | 18 | 1980 | 1401 |
| Probe | TET-5′-cctggagtgcagcctcaagttcgtc-3′- | 25 | 2017 | 1402 |
| | TAMRA | | | |
| Reverse | 5′-gtctcccgtgatcatgaaca-3′ | 20 | 2121 | 1403 |

TABLE BHD
General_screening_panel_v1.4
Column A - Rel. Exp.(%) Ag3287, Run 216516908

| Tissue Name | A | Tissue Name | A |
|---|---|---|---|
| Adipose | 4.8 | Renal ca.TK-10 | 26.4 |
| Melanoma* Hs688(A).T | 23.0 | Bladder | 19.9 |
| Melanoma* Hs688(B).T | 27.9 | Gastric ca. (liver met.) NCI-N87 | 44.4 |
| Melanoma* M14 | 31.6 | Gastric ca. KATO III | 39.8 |
| Melanoma* LOXIMVI | 33.7 | Colon ca. SW-948 | 24.3 |
| Melanoma* SK-MEL-5 | 31.0 | Colon ca. SW480 | 39.8 |
| Squamous cell carcinoma SCC-4 | 18.4 | Colon ca.* (SW480 met) SW620 | 33.0 |
| Testis Pool | 12.2 | Colon ca. HT29 | 10.0 |
| Prostate ca.* (bone met) PC-3 | 49.7 | Colon ca. HCT-116 | 48.3 |
| Prostate Pool | 6.8 | Colon ca. CaCo-2 | 46.7 |
| Placenta | 23.0 | Colon cancer tissue | 19.1 |
| Uterus Pool | 4.5 | Colon ca. SW1116 | 11.7 |
| Ovarian ca. OVCAR-3 | 22.7 | Colon ca. Colo-205 | 15.9 |
| Ovarian ca. SK-OV-3 | 48.0 | Colon ca. SW-48 | 10.9 |
| Ovarian ca. OVCAR-4 | 20.6 | Colon Pool | 16.5 |
| Ovarian ca. OVCAR-5 | 38.4 | Small Intestine Pool | 14.5 |
| Ovarian ca. IGROV-1 | 34.2 | Stomach Pool | 9.2 |
| Ovarian ca. OVCAR-8 | 13.5 | Bone Marrow Pool | 6.8 |
| Ovary | 11.7 | Fetal Heart | 18.0 |
| Breast ca. MCF-7 | 33.0 | Heart Pool | 5.2 |
| Breast ca. MDA-MB-231 | 49.3 | Lymph Node Pool | 19.8 |
| Breast ca. BT 549 | 100.0 | Fetal Skeletal Muscle | 10.5 |
| Breast ca. T47D | 76.8 | Skeletal Muscle Pool | 5.8 |
| Breast ca. MDA-N | 24.0 | Spleen Pool | 12.0 |
| Breast Pool | 18.4 | Thymus Pool | 19.1 |
| Trachea | 16.6 | CNS cancer (glio/astro) U87-MG | 36.1 |
| Lung | 5.5 | CNS cancer (glio/astro) U-118-MG | 65.5 |
| Fetal Lung | 42.6 | CNS cancer (neuro; met) SK-N-AS | 69.3 |
| Lung ca. NCI-N417 | 18.3 | CNS cancer (astro) SF-539 | 22.4 |
| Lung ca. LX-1 | 26.1 | CNS cancer (astro) SNB-75 | 49.0 |
| Lung ca. NCI-H146 | 17.8 | CNS cancer (glio) SNB-19 | 31.9 |
| Lung ca. SHP-77 | 37.9 | CNS cancer (glio) SF-295 | 84.7 |
| Lung ca. A549 | 24.1 | Brain (Amygdala) Pool | 7.5 |
| Lung ca. NCI-H526 | 7.3 | Brain (cerebellum) | 42.3 |
| Lung ca. NCI-H23 | 41.8 | Brain (fetal) | 26.8 |
| Lung ca. NCI-H460 | 34.6 | Brain (Hippocampus) Pool | 8.2 |
| Lung ca. HOP-62 | 13.4 | Cerebral Cortex Pool | 9.1 |
| Lung ca. NCI-H522 | 17.6 | Brain (Substantia nigra) Pool | 9.1 |
| Liver | 5.3 | Brain (Thalamus) Pool | 13.1 |
| Fetal Liver | 21.6 | Brain (whole) | 18.4 |
| Liver ca. HepG2 | 23.0 | Spinal Cord Pool | 6.6 |
| Kidney Pool | 22.1 | Adrenal Gland | 24.0 |
| Fetal Kidney | 24.1 | Pituitary gland Pool | 6.0 |
| Renal ca. 786-0 | 20.9 | Salivary Gland | 13.3 |
| Renal ca. A498 | 23.2 | Thyroid (female) | 9.5 |
| Renal ca. ACHN | 12.4 | Pancreatic ca. CAPAN2 | 44.8 |
| Renal ca. UO-31 | 27.7 | Pancreas Pool | 21.6 |

TABLE BHE
Panel 4D
Column A - Rel. Exp. (%) Ag1179, Run 139820117
Column B - Rel. Exp. (%) Ag3287, Run 164633941

| Tissue Name | A | B | Tissue Name | A | B |
|---|---|---|---|---|---|
| Secondary Th1 act | 46.0 | 55.5 | HUVEC IL-1 beta | 5.9 | 9.9 |
| Secondary Th2 act | 76.3 | 71.7 | HUVEC IFN gamma | 21.3 | 65.1 |
| Secondary Tr1 act | 38.4 | 49.7 | HUVEC TNF alpha + IFN gamma | 10.7 | 25.9 |
| Secondary Th1 rest | 15.0 | 18.2 | HUVEC TNF alpha + IL4 | 17.1 | 23.5 |
| Secondary Th2 rest | 18.8 | 18.8 | HUVEC IL-11 | 9.3 | 14.4 |
| Secondary Tr1 rest | 10.7 | 23.5 | Lung Microvascular EC none | 17.6 | 26.8 |
| Primary Th1 act | 59.5 | 52.1 | Lung Microvascular EC TNF alpha + | 10.9 | 21.0 |
| | | | IL-1 beta | | |
| Primary Th2 act | 47.0 | 40.1 | Microvascular Dermal EC none | 37.9 | 26.1 |
| Primary Tr1 act | 75.3 | 68.8 | Microsvasular Dermal EC TNF alpha + | 21.3 | 17.7 |
| | | | IL-1 beta | | |
| Primary Th1 rest | 52.1 | 60.3 | Bronchial epithelium TNF alpha + | 32.5 | 33.4 |
| | | | IL1 beta | | |
| Primary Th2 rest | 30.1 | 33.9 | Small airway epithelium none | 11.9 | 19.8 |
| Primary Tr1 rest | 25.3 | 25.7 | Small airway epithelium TNF alpha + | 58.6 | 73.2 |
| | | | IL-1 beta | | |
| CD4SRA CD4 lymphocyte | 25.0 | 40.1 | Coronery artery SMC rest | 15.6 | 22.5 |
| act | | | | | |
| CD45RO CD4 lymphocyte | 56.3 | 63.7 | Coronery artery SMC TNF alpha + | 14.9 | 13.6 |
| act | | | IL-1 beta | | |
| CD8 lymphocyte act | 35.6 | 53.6 | Astrocytes rest | 15.2 | 21.8 |
| Secondary CD8 | 44.4 | 48.3 | Astrocytes TNF alpha + IL-1 beta | 17.3 | 19.6 |
| lymphocyte rest | | | | | |
| Secondary CD8 | 29.1 | 21.3 | KU-812 (Basophil) rest | 39.0 | 59.9 |
| lymphocyte act | | | | | |
| CD4 lymphocyte none | 16.5 | 22.5 | KU-812 (Basophil) PMA/ionomycin | 83.5 | 83.5 |
| 2ry Th1/Th2/Tr1 anti- | 31.4 | 25.2 | CCD1106 (Keratinocytes) none | 16.4 | 24.1 |
| CD95 CH11 | | | | | |
| LAK cells rest | 40.6 | 43.8 | 93580 CCD1106 (Keratinocytes) | 54.7 | 25.0 |
| | | | TNFa and IFNg | | |
| LAK cells IL-2 | 37.1 | 42.6 | Liver cirrhosis | 6.0 | 6.8 |
| LAK cells IL-2 + IL-12 | 35.8 | 34.2 | Lupus kidney | 17.1 | 10.4 |
| LAK cells IL-2 + IFN | 39.8 | 42.0 | NCI-H292 none | 29.5 | 35.4 |
| gamma | | | | | |
| LAK cells IL-2 + IL-18 | 28.3 | 42.0 | NCI-H292 IL-4 | 41.8 | 73.2 |
| LAK cells PMA/ionomycin | 18.4 | 19.8 | NCI-H292 IL-9 | 35.4 | 48.6 |
| NK Cells IL-2 rest | 18.3 | 26.2 | NCI-H292 IL-13 | 39.8 | 40.9 |
| Two Way MLR 3 day | 27.9 | 50.0 | NCI-H292 IFN gamma | 24.5 | 48.3 |
| Two Way MLR 5 day | 21.9 | 29.5 | HPAEC none | 20.0 | 29.5 |
| Two Way MLR 7 day | 19.3 | 19.6 | HPAEC TNF alpha + IL-1 beta | 18.9 | 23.7 |
| PBMC rest | 10.8 | 11.5 | Lung fibroblast none | 20.0 | 22.8 |
| PBMC PWM | 100.0 | 94.6 | Lung fibroblast TNF alpha + IL-1 | 9.9 | 15.5 |
| | | | beta | | |
| PBMC PHA-L | 62.0 | 49.0 | Lung fibroblast IL-4 | 26.2 | 42.0 |
| Ramos (B cell) none | 69.7 | 39.8 | Lung fibroblast IL-9 | 19.3 | 28.5 |
| Ramos (B cell) ionomycin | 93.3 | 100.0 | Lung fibroblast IL-13 | 41.5 | 27.9 |
| B lymphocytes PWM | 79.6 | 91.4 | Lung fibroblast IFN gamma | 34.6 | 43.5 |
| B lymphocytes CD40L and | 40.6 | 59.0 | Dermal fibroblast CCD1070 rest | 47.3 | 48.3 |
| IL-4 | | | | | |
| EOL-1 dbcAMP | 43.2 | 76.3 | Dermal fibroblast CCD1070 TNF | 47.6 | 67.4 |
| | | | alpha | | |
| EOL-1 dbcAMP | 28.9 | 23.8 | Dermal fibroblast CCD1070 IL-1 | 31.4 | 34.6 |
| PMA/ionomycin | | | beta | | |
| Dendritic cells none | 30.8 | 36.3 | Dermal fibroblast IFN gamma | 10.2 | 18.6 |
| Dendritic cells LPS | 32.8 | 44.4 | Dermal fibroblast IL-4 | 23.7 | 24.8 |
| Dendritic cells anti-CD40 | 35.6 | 38.4 | IBD Colitis 2 | 2.6 | 2.8 |
| Monocytes rest | 28.1 | 37.9 | IBD Crohn's | 1.1 | 1.4 |
| Monocytes LPS | 37.9 | 24.3 | Colon | 17.0 | 23.7 |
| Macrophages rest | 58.2 | 67.4 | Lung | 13.9 | 21.6 |
| Macrophages LPS | 69.3 | 40.9 | Thymus | 39.0 | 31.6 |
| HUVEC none | 18.7 | 23.5 | Kidney | 40.6 | 63.3 |
| HUVEC starved | 24.8 | 34.9 | | | |

TABLE BHF
general oncology screening panel_v_2.4
Column A - Rel. Exp.(%) Ag3287, Run 268695261

| Tissue Name | A |
|---|---|
| Colon cancer 1 | 36.1 |
| Colon NAT 1 | 11.3 |
| Colon cancer 2 | 34.6 |
| Colon NAT 2 | 20.0 |
| Colon cancer 3 | 100.0 |
| Colon NAT 3 | 22.2 |
| Colon malignant cancer 4 | 100.0 |
| Colon NAT 4 | 15.1 |
| Lung cancer 1 | 48.6 |
| Lung NAT 1 | 5.4 |
| Lung cancer 2 | 95.9 |
| Lung NAT 2 | 4.6 |
| Squamous cell carcinoma 3 | 51.4 |
| Lung NAT 3 | 3.5 |
| Metastatic melanoma 1 | 28.9 |
| Melanoma 2 | 5.6 |
| Melanoma 3 | 4.9 |
| Metastatic melanoma 4 | 76.8 |
| Metastatic melanoma 5 | 59.5 |
| Bladder cancer 1 | 2.4 |
| Bladder NAT 1 | 0.0 |
| Bladder cancer 2 | 9.5 |
| Bladder NAT 2 | 0.8 |
| Bladder NAT 3 | 1.4 |
| Bladder NAT 4 | 5.3 |
| Prostate adenocarcinoma 1 | 30.1 |
| Prostate adenocarcinoma 2 | 6.3 |
| Prostate adenocarcinoma 3 | 15.6 |
| Prostate adenocarcinoma 4 | 21.6 |
| Prostate NAT 5 | 14.3 |
| Prostate adenocarcinoma 6 | 12.4 |
| Prostate adenocarcinoma 7 | 10.8 |
| Prostate adenocarcinoma 8 | 3.8 |
| Prostate adenocarcinoma 9 | 31.9 |
| Prostate NAT 10 | 3.3 |
| Kidney cancer 1 | 31.9 |
| Kidney NAT 1 | 21.8 |
| Kidney cancer 2 | 78.5 |
| Kidney NAT 2 | 23.5 |
| Kidney cancer 3 | 30.6 |
| Kidney NAT 3 | 7.7 |
| Kidney cancer 4 | 16.0 |
| Kidney NAT 4 | 11.3 |

General_screening_panel_v1.4 Summary: Ag3287—This gene showed moderate to high expression in all samples on this panel, with the highest level of expression in breast cancer cell line BT 549 (CT=25.0). The widespread expression of this gene indicates that the gene product may be involved in cell differentiation and growth.

This gene was widely expressed among tissues with metabolic function, including adipose, adult and fetal skeletal muscle and heart, the pancreas, fetal liver, and the adrenal, thyroid, and pituitary glands. This expression profile indicates that this gene product is involved in metabolic function. Therapeutic modulation of this gene, expressed protein and/or use of antibodies or small molecule drugs targeting the gene or gene product are useful in the treatment of metabolic disorders, such as obesity and diabetes.

This gene showed widespread expression of this gene in the brain. This indicates that the protein encoded by this gene is important for normal neurological function. Therapeutic modulation of this gene, expressed protein and/or use of antibodies or small molecule drugs targeting the gene or gene product are useful in the treatment of neurodegenerative disorders, such as Alzheimer's disease and Parkinson's disease.

Panel 4D Summary: Ag1179/Ag3287 This gene was expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression indicates that this gene product may be involved in homeostatic processes for these and other cell types and tissues.

This pattern is in agreement with the expression profile in General_screening_panel_v1.5 and also indicates a role for the gene product in cell survival and proliferation.

Therapeutic modulation of this gene, expressed protein and/or use of antibodies or small molecule drugs targeting the gene or gene product are useful in the treatment of autoimmune and inflammatory diseases such as asthma, allergies, inflammatory bowel disease, lupus erythematosus, psoriasis, rheumatoid arthritis, and osteoarthritis.

general oncology screening panel_v_{—}2.4 Summary: Ag3287 Highest expression was detected in a colon cancer sample (CT=26.4), with prominent expression seen in squamous cell carcinoma and melanoma samples. This gene was overexpressed in colon and lung cancers when compared to expression in the normal adjacent tissues. Expression of this gene is useful as a marker of colon and lung cancers. Therapeutic modulation of this gene, expressed protein and/or use of antibodies or small molecule drugs targeting the gene or gene product are useful in the treatment of colon and lung cancers.

BI. CG57758-03: Renal Sodium/Dicarboxylate Cotransporter.

Expression of gene CG57758-03 was assessed using the primer-probe sets Ag3326 and Ag3692, described in Tables BIA and BIB. Results of the RTQ-PCR runs are shown in Tables BIC, BID and BIE. CG57758-03 represents a full-length physical clone of the CG57758-01 gene.

TABLE BIA
Probe Name Ag3326

| | | | | SEQ |
|---|---|---|---|---|
| | | | Start | ID |
| Primers | Sequences | Length | Position | No |
| Forward | 5′-ccatttactggtgcacagaagt-3′ | 22 | 138 | 1404 |
| Probe | TET-5′-atccctctggctgtcacctctctcat-3′- | 26 | 161 | 1405 |
| | TAMRA | | | |
| Reverse | 5′-ggagtccagaatctggaagagt-3′ | 22 | 205 | 1406 |

TABLE BIB
Probe Name Ag3692

| | | | | SEQ |
|---|---|---|---|---|
| | | | Start | ID |
| Primers | Sequences | Length | Position | No |
| Forward | 5′-ccatttactggtgcacagaagt-3′ | 22 | 138 | 1407 |
| Probe | TET-5′-atccctctggctgtcacctctctcat-3′- | 26 | 161 | 1408 |
| | TAMRA | | | |
| Reverse | 5′-ggagtccagaatctggaagagt-3′ | 22 | 205 | 1409 |

TABLE BIC
General_screening_panel_v1.4
Column A - Rel. Exp. (%) Ag3326, Run 215678613
Column B - Rel. Exp. (%) Ag3692, Run 217131191

| Tissue Name | A | B | Tissue Name | A | B |
|---|---|---|---|---|---|
| Adipose | 0.0 | 0.0 | Renal Ca. TK-10 | 11.4 | 12.0 |
| Melanoma* Hs688(A).T | 0.0 | 0.0 | Bladder | 0.0 | 0.1 |
| Melanoma* Hs688(B).T | 0.1 | 0.0 | Gastric ca. (liver met.) NCI-N87 | 0.0 | 0.0 |
| Melanoma* M14 | 0.0 | 0.0 | Gastric ca. KATO III | 0.0 | 0.0 |
| Melanoma* LOXIMVI | 0.0 | 0.0 | Colon ca. SW-948 | 0.0 | 0.0 |
| Melanoma* SK-MEL-5 | 0.0 | 0.0 | Colon ca. SW480 | 0.0 | 0.0 |
| Squamous cell carcinoma | 0.9 | 0.7 | Colon ca.* (SW480 met) SW620 | 0.0 | 0.0 |
| SCC-4 | | | | | |
| Testis Pool | 0.1 | 0.2 | Colon ca. HT29 | 0.0 | 0.0 |
| Prostate ca.* (bone met) PC-3 | 0.0 | 0.0 | Colon ca. HCT-116 | 0.0 | 0.0 |
| Prostate Pool | 0.0 | 0.0 | Colon ca. CaCo-2 | 0.0 | 0.0 |
| Placenta | 0.0 | 0.0 | Colon cancer tissue | 0.1 | 0.0 |
| Uterus Pool | 0.0 | 0.0 | Colon ca. SW1116 | 0.0 | 0.0 |
| Ovarian ca. OVCAR-3 | 0.0 | 0.0 | Colon ca. Colo-205 | 0.0 | 0.0 |
| Ovarian ca. SK-OV-3 | 0.0 | 0.0 | Colon ca. SW-48 | 0.0 | 0.0 |
| Ovarian ca. OVCAR-4 | 0.1 | 0.0 | Colon Pool | 0.6 | 0.0 |
| Ovarian ca. OVCAR-5 | 0.0 | 0.0 | Small Intestine Pool | 0.1 | 0.0 |
| Ovarian ca. IGROV-1 | 0.0 | 0.0 | Stomach Pool | 0.0 | 0.0 |
| Ovarian ca. OVCAR-8 | 2.8 | 2.2 | Bone Marrow Pool | 0.0 | 0.1 |
| Ovary | 0.7 | 0.6 | Fetal Heart | 0.0 | 0.0 |
| Breast ca. MCF-7 | 0.0 | 0.0 | Heart Pool | 0.0 | 0.0 |
| Breast ca. MDA-MB-231 | 0.0 | 0.0 | Lymph Node Pool | 0.1 | 0.0 |
| Breast ca. BT 549 | 0.6 | 0.8 | Fetal Skeletal Muscle | 0.0 | 0.0 |
| Breast ca. T47D | 0.0 | 0.0 | Skeletal Muscle Pool | 0.0 | 0.0 |
| Breast ca. MDA-N | 0.0 | 0.0 | Spleen Pool | 0.4 | 0.2 |
| Breast Pool | 0.0 | 0.1 | Thymus Pool | 0.0 | 0.0 |
| Trachea | 0.2 | 0.1 | CNS cancer (glio/astro) U87-MG | 0.0 | 0.0 |
| Lung | 0.0 | 0.0 | CNS cancer (glio/astro) U-118- | 0.0 | 0.0 |
| | | | MG | | |
| Fetal Lung | 0.2 | 0.1 | CNS cancer (neuro; met) SK-N- | 0.0 | 0.0 |
| | | | AS | | |
| Lung ca. NCI-N417 | 0.0 | 0.0 | CNS cancer (astro) SF-539 | 0.0 | 0.0 |
| Lung ca. LX-1 | 0.0 | 0.0 | CNS cancer (astro) SNB-75 | 0.0 | 0.0 |
| Lung ca. NCI-H146 | 0.0 | 0.0 | CNS cancer (glio) SNB-19 | 0.0 | 0.0 |
| Lung ca. SHP-77 | 0.0 | 0.0 | CNS cancer (glio) SF-295 | 0.1 | 0.1 |
| Lung ca. A549 | 0.0 | 0.1 | Brain (Amygdala) Pool | 0.4 | 0.4 |
| Lung ca. NCI-H526 | 2.0 | 0.0 | Brain (cerebellum) | 1.4 | 1.0 |
| Lung ca. NCI-H23 | 0.7 | 0.6 | Brain (fetal) | 0.7 | 0.4 |
| Lung ca. NCI-H460 | 0.0 | 0.0 | Brain (Hippocampus) Pool | 0.5 | 0.7 |
| Lung ca. HOP-62 | 0.1 | 0.2 | Cerebral Cortex Pool | 1.4 | 1.5 |
| Lung ca. NCI-H522 | 0.0 | 0.0 | Brain (Substantia nigra) Pool | 1.4 | 1.4 |
| Liver | 28.7 | 24.1 | Brain (Thalamus) Pool | 1.1 | 0.9 |
| Fetal Liver | 100.0 | 100.0 | Brain (whole) | 4.1 | 3.7 |
| Liver ca. HepG2 | 29.5 | 26.2 | Spinal Cord Pool | 0.1 | 0.2 |
| Kidney Pool | 0.0 | 0.0 | Adrenal Gland | 2.6 | 1.9 |
| Fetal Kidney | 0.1 | 0.1 | Pituitary gland Pool | 0.0 | 0.2 |
| Renal ca. 786-0 | 0.0 | 0.0 | Salivary Gland | 40.9 | 35.1 |
| Renal ca. A498 | 0.0 | 0.0 | Thyroid (female) | 0.0 | 0.0 |
| Renal ca. ACHN | 0.0 | 0.0 | Pancreatic ca. CAPAN2 | 0.5 | 0.8 |
| Renal ca. UO-31 | 0.0 | 0.0 | Pancreas Pool | 0.0 | 0.0 |

TABLE BID
Panel 4.1D
Column A - Rel. Exp.(%) Ag3692, Run 169987356

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 11.3 |
| Primary Tr1 act | 4.2 | Microsvasular Dermal EC TNFalpha + IL- | 0.0 |
| | | 1beta | |
| Primary Th1 rest | 0.0 | Bronchial epithelium TNFalpha + IL1beta | 28.5 |
| Primary Th2 rest | 0.0 | Small airway epithelium none | 5.7 |
| Primary Tr1 rest | 0.0 | Small airway epithelium TNFalpha + IL- | 0.0 |
| | | 1beta | |
| CD45RA CD4 lymphocyte act | 3.9 | Coronery artery SMC rest | 0.0 |
| CD45RO CD4 lymphocyte act | 0.0 | Coronery artery SMC TNFalpha + IL-1beta | 0.0 |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 3.6 |
| CD4 lymphocyte none | 0.0 | KU-812 (Basophil) PMA/ionomycin | 4.3 |
| 2ry Th1/Th2/Tr1 anti-CD95 | 0.0 | CCD1106 (Keratinocytes) none | 10.7 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 94.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 3.2 | HPAEC TNF alpha + IL-1 beta | 0.0 |
| Two Way MLR 7 day | 0.0 | Lung fibroblast none | 0.0 |
| PBMC rest | 0.0 | Lung fibroblast TNF alpha + IL-1 beta | 0.0 |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and IL-4 | 0.0 | Dermal fibroblast CCD1070 TNF alpha | 0.0 |
| EOL-1 dbcAMP | 0.0 | Dermal fibroblast CCD1070 IL-1 beta | 0.0 |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 2.4 |
| HUVEC none | 0.0 | Kidney | 100.0 |
| HUVEC starved | 0.0 | | |

General_screening_panel_v1.4 Summary: This gene was highly expressed in fetal liver (CT=26.5-27.0) and moderately expressed in adult liver and liver cancer cell line HepG2. This result agrees with the results seen in Panel 5 (expression in HepG2). These results are in agreement with published data that show a novel sodium dicarboxylate transporter in brain, choroid plexus kidney, intestine and liver (Chen X Z, Shayakul C, Berger U V, Tian W, Hediger M A. Characterization of a rat Na+-dicarboxylate cotransporter. J Biol Chem 1998 Aug. 14; 273(33):20972-81; Pajor A M, Gangula R, Yao X. Cloning and functional characterization of a high-affinity Na(+)/dicarboxylate cotransporter from mouse brain. Am J Physiol Cell Physiol 2001 May; 280(5):C 1215-23). Expression of this gene is useful as a marker for liver derived tissue.

This gene was expressed at low levels throughout the CNS, including in amygdala, substantia nigra, thalamus, cerebellum, and cerebral cortex. Therapeutic modulation of this gene, expressed protein and/or use of antibodies or small molecule drugs targeting the gene or gene product are useful in the treatment of central nervous system disorders such as Parkinson's disease, epilepsy, multiple sclerosis, schizophrenia and depression.

Low but significant levels of expression were also seen in the adrenal gland. Therapeutic modulation of this gene, expressed protein and/or use of antibodies or small molecule drugs targeting the gene or gene product are useful in the treatment of metabolic disorders of the adrenal gland, including adrenoleukodystrophy and congenital adrenal hyperplasia.

Panel 4.1D Summary: Ag3692 Significant expression of this gene was seen only in kidney and a liver cirrhosis sample (CTs=34.0). These results confirm that this gene was expressed in liver derived samples. The presence in the kidney was also in agreement with published results. Please see Panel 1.4. Therapeutic modulation of this gene, expressed protein and/or use of antibodies or small molecule drugs targeting the gene or gene product are useful in the treatment of kidney inflammation.

BJ. CG58504-01: ADAMTS12.

Expression of gene CG58504-01 was assessed using the primer-probe set Ag2475, described in Table BJA. Results of the RTQ-PCR runs are shown in Tables BJB, BJC, BJD, BJE and BJF.

TABLE BJA
Probe Name Ag2475

| | | | | SEQ |
|---|---|---|---|---|
| | | | Start | ID |
| Primers | Sequences | Length | Position | No |
| Forward | 5′-agagtgacctcaatcctgttca-3′ | 22 | 1318 | 1410 |
| Probe | TET-5′-acgtggctgtccttctcaccagaaag-3′- | 26 | 1345 | 1411 |
| | TAMRA | | | |
| Reverse | 5′-gattgaaaccagcacagatgtc-3′ | 22 | 1371 | 1412 |

TABLE BJB
HASS Panel v1.0
Column A - Rel. Exp.(%) Ag2475, Run 268366853

| Tissue Name | A |
|---|---|
| MGF-7 C1 | 0.1 |
| MCF-7 C2 | 0.0 |
| MCF-7 C3 | 0.0 |
| MCF-7 C4 | 0.0 |
| MCF-7 C5 | 0.0 |
| MCF-7 C6 | 0.0 |
| MCF-7 C7 | 0.0 |
| MCF-7 C9 | 0.0 |
| MCF-7 C10 | 0.0 |
| MCF-7 C11 | 0.0 |
| MCF-7 C12 | 0.0 |
| MCF-7 C13 | 0.0 |
| MCF-7 C15 | 0.0 |
| MCF-7 C16 | 0.0 |
| MCF-7 C17 | 0.0 |
| T24 D1 | 16.2 |
| T24 D2 | 14.4 |
| T24 D3 | 40.1 |
| T24 D4 | 28.1 |
| T24 D5 | 31.4 |
| T24 D6 | 23.2 |
| T24 D7 | 8.0 |
| T24 D9 | 7.2 |
| T24 D10 | 10.3 |
| T24 D11 | 9.5 |
| T24 D12 | 14.8 |
| T24 D13 | 3.8 |
| T24 D15 | 4.6 |
| T24 D16 | 6.7 |
| T24 D17 | 13.0 |
| CAPaN B1 | 0.0 |
| CAPaN B2 | 0.0 |
| CAPaN B3 | 0.1 |
| CAPaN B4 | 0.2 |
| CAPaN B5 | 0.1 |
| CAPaN B6 | 0.2 |
| CAPaN B7 | 0.0 |
| CAPaN B8 | 0.1 |
| CAPaN B9 | 0.1 |
| CAPaN B10 | 0.4 |
| CAPaN B11 | 0.1 |
| CAPaN B12 | 0.3 |
| CAPaN B13 | 0.1 |
| CAPaN B14 | 0.0 |
| CAPaN B15 | 0.0 |
| CAPaN B16 | 0.2 |
| CAPaN B17 | 0.3 |
| U87-MG F1 (B) | 17.3 |
| U87-MG F2 | 12.9 |
| U87-MG F3 | 17.4 |
| U87-MG F4 | 27.4 |
| U87-MG F5 | 66.0 |
| U87-MG F6 | 84.7 |
| U87-MG F7 | 9.2 |
| U87-MG F8 | 10.6 |
| U87-MG F9 | 5.4 |
| U87-MG F10 | 61.1 |
| U87-MG F11 | 87.7 |
| U87-MG F12 | 45.7 |
| U87-MG F13 | 15.7 |
| U87-MG F14 | 25.2 |
| U87-MG F15 | 16.3 |
| U87-MG F16 | 56.6 |
| U87-MG F17 | 73.2 |
| LnCAP A1 | 0.0 |
| LnCAP A2 | 0.0 |
| LnCAP A3 | 0.0 |
| LnCAP A4 | 0.1 |
| LnGAP A5 | 0.0 |
| LnCAP A6 | 0.0 |
| LnCAP A7 | 0.0 |
| LnCAP A8 | 0.0 |
| LnCAP A9 | 0.1 |
| LnCAP A10 | 0.0 |
| LnCAP A11 | 0.0 |
| LnCAP A12 | 0.0 |
| LnCAP A13 | 0.1 |
| LnCAP A14 | 0.0 |
| LnCAP A15 | 0.0 |
| LnCAP A16 | 0.0 |
| LnCAP A17 | 0.1 |
| Primary Astrocytes | 100.0 |
| Primary Renal Proximal Tubule Epithelial cell A2 | 4.4 |
| Primary melanocytes A5 | 4.5 |
| 126443 - 341 medullo | 0.0 |
| 126444 - 487 medullo | 0.0 |
| 126445 - 425 medullo | 0.3 |
| 126446 - 690 medullo | 1.0 |
| 126447 - 54 adult glioma | 17.6 |
| 126448 - 245 adult glioma | 13.2 |
| 126449 - 317 adult glioma | 0.2 |
| 126450 - 212 glioma | 4.4 |
| 126451 - 456 glioma | 0.3 |

TABLE BJC
Panel 1.3D
Column A - Rel. Exp.(%) Ag2475, Run 162401130

| Tissue Name | A | Tissue Name | A |
|---|---|---|---|
| Liver adenocarcinoma | 0.0 | Kidney (fetal) | 5.4 |
| Pancreas | 0.0 | Renal ca. 786-0 | 0.0 |
| Pancreatic ca. CAPAN 2 | 0.1 | Renal ca. A498 | 8.1 |
| Adrenal gland | 0.5 | Renal ca. RXF 393 | 5.5 |
| Thyroid | 0.0 | Renal ca. ACHN | 0.0 |
| Salivary gland | 0.0 | Renal ca. UO-31 | 23.8 |
| Pituitary gland | 0.0 | Renal ca. TK-10 | 1.5 |
| Brain (fetal) | 0.0 | Liver | 0.2 |
| Brain (whole) | 0.1 | Liver (fetal) | 0.8 |
| Brain (amygdala) | 0.0 | Liver ca. (hepatoblast) HepG2 | 0.0 |
| Brain (cerebellum) | 0.0 | Lung | 1.9 |
| Brain (hippocampus) | 0.0 | Lung (fetal) | 4.3 |
| Brain (substantia nigra) | 0.1 | Lung ca. (small cell) LX-1 | 2.0 |
| Brain (thalamus) | 0.5 | Lung ca. (small cell) NCI-H69 | 0.0 |
| Cerebral Cortex | 0.3 | Lung ca. (s. cell var.) SHP-77 | 0.0 |
| Spinal cord | 0.2 | Lung ca. (large cell)NCI-H460 | 0.2 |
| glio/astro U87-MG | 14.9 | Lung ca. (non-sm. cell) A549 | 0.3 |
| glio/astro U-118-MG | 2.6 | Lung ca. (non-s. cell) NCI-H23 | 0.0 |
| astrocytoma SW1783 | 67.8 | Lung ca. (non-s. cell) HOP-62 | 37.1 |
| neuro*; met SK-N-AS | 0.0 | Lung ca. (non-s. cl) NCI-H522 | 0.0 |
| astrocytoma SF-539 | 2.9 | Lung ca. (squam.) SW 900 | 0.0 |
| astrocytoma SNB-75 | 6.5 | Lung ca. (squam.) NCI-H596 | 0.0 |
| glioma SNB-19 | 1.1 | Mammary gland | 3.9 |
| glioma U251 | 0.7 | Breast ca.* (pl. ef) MCF-7 | 0.0 |
| glioma SF-295 | 0.4 | Breast ca.* (pl. ef) MDA-MB-231 | 17.1 |
| Heart (Fetal) | 7.7 | Breast ca.* (p1. ef) T47D | 0.0 |
| Heart | 0.5 | Breast ca. BT-549 | 6.0 |
| Skeletal muscle (Fetal) | 100.0 | Breast ca. MDA-N | 0.0 |
| Skeletal muscle | 0.3 | Ovary | 13.7 |
| Bone marrow | 0.0 | Ovarian ca. OVCAR-3 | 0.0 |
| Thymus | 0.3 | Ovarian ca. OVCAR-4 | 0.0 |
| Spleen | 0.2 | Ovarian ca. OVCAR-5 | 0.0 |
| Lymph node | 0.0 | Ovarian ca. OVCAR-8 | 3.5 |
| Colorectal | 1.3 | Ovarian ca. IGROV-1 | 0.0 |
| Stomach | 0.3 | Ovarian ca. (ascites) SK-OV-3 | 0.5 |
| Small intestine | 0.4 | Uterus | 0.4 |
| Colon ca. SW480 | 0.0 | Placenta | 2.0 |
| Colon ca.* SW620 (SW480 met) | 0.3 | Prostate | 0.2 |
| Colon ca. HT29 | 0.0 | Prostate ca.* (bone met) PC-3 | 0.0 |
| Colon ca. HCT-116 | 0.0 | Testis | 1.3 |
| Colon ca. CaCo-2 | 0.0 | Melanoma Hs688(A).T | 9.5 |
| CC Well to Mod Diff(ODO3866) | 24.8 | Melanoma* (met) Hs688(B).T | 22.1 |
| Colon ca. HCC-2998 | 0.0 | Melanoma UACC-62 | 0.0 |
| Gastric ca. (liver met) NCI-N87 | 0.0 | Melanoma M14 | 0.0 |
| Bladder | 6.7 | Melanoma LOX IMVI | 1.8 |
| Trachea | 0.1 | Melanoma (met) SK-MEL-5 | 0.0 |
| Kidney | 0.2 | Adipose | 7.9 |

TABLE BJD
Panel 2D
Column A - Rel. Exp.(%) Ag2475, Run 165296233

| Tissue Name | A | Tissue Name | A |
|---|---|---|---|
| Normal Colon | 21.2 | Kidney Margin 8120608 | 1.2 |
| CC Well to Mod Diff(ODO3866) | 40.3 | Kidney Cancer 8120613 | 2.8 |
| CC Margin (ODO3866) | 4.7 | Kidney Margin 8120614 | 4.1 |
| CC Gr.2 rectosigmoid (ODO3868) | 29.1 | Kidney Cancer 9010320 | 42.6 |
| CC Margin (ODO3868) | 4.7 | Kidney Margin 9010321 | 11.3 |
| CC Mod Diff (ODO3920) | 8.8 | Normal Uterus | 6.9 |
| CC Margin (ODO3920) | 6.6 | Uterine Cancer 064011 | 27.5 |
| CC Gr.2 ascend colon (ODO3921) | 60.3 | Normal Thyroid | 2.2 |
| CC Margin (ODO3921) | 13.6 | Thyroid Cancer | 0.8 |
| CC from Partial Hepatectomy | 53.2 | Thyroid Cancer A302152 | 7.4 |
| (ODO4309) Mets | | | |
| Liver Margin (ODO4309) | 6.7 | Thyroid Margin A302153 | 6.3 |
| Colon mets to lung (OD04451-01) | 18.3 | Normal Breast | 59.9 |
| Lung Margin (OD04451-02) | 5.8 | Breast Cancer | 42.0 |
| Normal Prostate 6546-1 | 1.0 | Breast Cancer (OD04590-01) | 51.4 |
| Prostate Cancer (OD04410) | 7.4 | Breast Cancer Mets (OD04590-03) | 65.1 |
| Prostate Margin (OD04410) | 14.5 | Breast Cancer Metastasis | 4.3 |
| Prostate Cancer (OD04720-01) | 7.1 | Breast Cancer | 67.8 |
| Prostate Margin (OD04720-02) | 14.2 | Breast Cancer | 86.5 |
| Normal Lung | 23.0 | Breast Cancer 9100266 | 19.6 |
| Lung Met to Muscle (ODO4286) | 15.5 | Breast Margin 9100265 | 48.3 |
| Muscle Margin (ODO4286) | 9.7 | Breast Cancer A209073 | 93.3 |
| Lung Malignant Cancer (OD03126) | 100.0 | Breast Margin A209073 | 53.6 |
| Lung Margin (OD03126) | 27.2 | Normal Liver | 5.8 |
| Lung Cancer (OD04404) | 78.5 | Liver Cancer | 1.5 |
| Lung Margin (OD04404) | 25.3 | Liver Cancer 1025 | 4.8 |
| Lung Cancer (OD04565) | 54.7 | Liver Cancer 1026 | 16.5 |
| Lung Margin (OD04565) | 24.0 | Liver Cancer 6004-T | 5.7 |
| Lung Cancer (OD04237-01) | 54.7 | Liver Tissue 6004-N | 5.9 |
| Lung Margin (OD04237-02) | 38.7 | Liver Cancer 6005-T | 14.0 |
| Ocular Mel Met to Liver (ODO4310) | 0.4 | Liver Tissue 6005-N | 5.9 |
| Liver Margin (ODO4310) | 10.8 | Normal Bladder | 32.3 |
| Melanoma Metastasis | 4.7 | Bladder Cancer | 29.7 |
| Lung Margin (OD04321) | 15.1 | Bladder Cancer | 15.0 |
| Normal Kidney | 19.2 | Bladder Cancer (OD04718-01) | 48.0 |
| Kidney Ca, Nuclear grade 2 (OD04338) | 3.1 | Bladder Normal Adjacent | 17.6 |
| | | (OD04718-03) | |
| Kidney Margin (OD04338) | 6.7 | Normal Ovary | 9.5 |
| Kidney Ca Nuclear grade 1/2 | 1.4 | Ovarian Cancer | 71.7 |
| (OD04339) | | | |
| Kidney Margin (OD04339) | 9.9 | Ovarian Cancer (OD04768-07) | 2.7 |
| Kidney Ca, Clear cell type (OD04340) | 4.9 | Ovary Margin (OD04768-08) | 10.2 |
| Kidney Margin (OD04340) | 9.9 | Normal Stomach | 9.0 |
| Kidney Ca, Nuclear grade 3 (OD04348) | 17.0 | Gastric Cancer 9060358 | 4.0 |
| Kidney Margin (OD04348) | 6.1 | Stomach Margin 9060359 | 2.5 |
| Kidney Cancer (OD04622-O1) | 9.2 | Gastric Cancer 9060395 | 17.2 |
| Kidney Margin (OD04622-03) | 0.8 | Stomach Margin 9060394 | 5.7 |
| Kidney Cancer (OD04450-01) | 2.5 | Gastric Cancer 9060397 | 56.6 |
| Kidney Margin (OD04450-03) | 9.2 | Stomach Margin 9060396 | 1.8 |
| Kidney Cancer 8120607 | 3.2 | Gastric Cancer 064005 | 22.1 |

TABLE BJF
Panel 4D
Column A - Rel. Exp.(%) Ag2475, Run 163583185

| Tissue Name | A | Tissue Name | A |
|---|---|---|---|
| Secondary Th1 act | 0.0 | HUVEC IL-1beta | 0.2 |
| Secondary Th2 act | 0.0 | HUVEC IFN gamma | 0.3 |
| Secondary Tr1 act | 0.0 | HUVEC TNF alpha + IFN gamma | 0.1 |
| Secondary Th1 rest | 0.0 | HUVEC TNF alpha + IL4 | 0.0 |
| Secondary Th2 rest | 0.0 | HUVEC IL-11 | 0.1 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 0.7 |
| Primary Tr1 act | 0.0 | Microsvasular Dermal EC TNFalpha + IL- | 0.2 |
| | | 1beta | |
| Primary Th1 rest | 0.1 | Bronchial epithelium TNFalpha + IL1beta | 0.0 |
| Primary Th2 rest | 0.0 | Small airway epithelium none | 1.8 |
| Primary Trl rest | 0.0 | Small airway epithelium TNFalpha + IL- | 0.1 |
| | | 1beta | |
| CD45RA CD4 lymphocyte act | 3.9 | Coronery artery SMC rest | 100.0 |
| CD45RO CD4 lymphocyte act | 0.0 | Coronery artery SMC TNFalpha + IL-1beta | 29.9 |
| CD8 lymphocyte act | 0.0 | Astrocytes rest | 22.2 |
| Secondary CD8 lymphocyte rest | 0.0 | Astrocytes TNFalpha + IL-1beta | 24.5 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.5 |
| CH11 | | | |
| LAK cells rest | 0.0 | CCD1106 (Keratinocytes) TNFalpha + IL- | 0.0 |
| | | 1beta | |
| LAK cells IL-2 | 0.0 | Liver cirrhosis | 1.9 |
| LAK cells IL-2 + IL-12 | 0.1 | Lupus kidney | 0.6 |
| LAK cells IL-2 + IFN gamma | 0.0 | NCI-H292 none | 1.7 |
| LAK cells IL-2 + IL-18 | 0.0 | NCI-H292 IL-4 | 1.9 |
| LAK cells PMA/ionomycin | 0.0 | NCI-H292 IL-9 | 6.2 |
| NK Cells IL-2 rest | 0.0 | NCI-H292 IL-13 | 1.9 |
| Two Way MLR 3 day | 0.0 | NCI-H292 IFN gamma | 0.9 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 4.3 |
| PBMC PWM | 0.0 | Lung fibroblast TNF alpha + IL-1 beta | 7.0 |
| PBMC PHA-L | 0.0 | Lung fibroblast IL-4 | 20.2 |
| Ramos (B cell) none | 0.0 | Lung fibroblast IL-9 | 13.4 |
| Ramos (B cell) ionomycin | 0.0 | Lung fibroblast IL-13 | 9.2 |
| B lymphocytes PWM | 0.0 | Lung fibroblast IFN gamma | 7.7 |
| B lymphocytes CD40L and IL-4 | 0.0 | Dermal fibroblast CCD1070 rest | 13.4 |
| EOL-1 dbcAMP | 0.0 | Dermal fibroblast CCD1070 TNF alpha | 51.4 |
| EOL-1 dbcAMP | 0.0 | Dermal fibroblast CCD1070 IL-1 beta | 12.9 |
| PMA/ionomycin | | | |
| Dendritic cells none | 0.0 | Dermal fibroblast IFN gamma | 3.0 |
| Dendritic cells LPS | 0.0 | Dermal fibroblast IL-4 | 37.9 |
| Dendritic cells anti-CD40 | 0.0 | IBD Colitis 2 | 0.0 |
| Monocytes rest | 0.0 | IBD Crohn's | 0.2 |
| Monocytes LPS | 0.0 | Colon | 1.9 |
| Macrophages rest | 0.0 | Lung | 20.7 |
| Macrophages LPS | 0.0 | Thymus | 0.6 |
| HUVEC none | 0.0 | Kidney | 0.1 |
| HUVEC starved | 0.1 | | |

HASS Panel v1.0 Summary: Ag2475 This gene was expressed in glioma samples and primary astrocytes in culture (highest expression CT=27.8) indicating a role in cell growth. Expression of this gene in U87-MG (a mixed glial/astrocytoma cell line) was repressed by reducing the oxygen content of the environment. Serum starvation of these cells induces expression. This effect was not observed in T24 (bladder cancer) cells and thus may reflect tissue specific regulation of this gene.

Panel 1.3D Summary: Ag2475 Highest expression of the CG58504-01 gene was seen in fetal skeletal muscle (CT=28.4). This expression was significantly higher than expression seen in the corresponding adult tissue (CT=36.9). In addition, the relative overexpression of this gene in fetal skeletal muscle indicates that the protein product may enhance muscular growth or development in the fetus and thus may also act in a regenerative capacity in the adult. Therapeutic modulation of this gene, expressed protein and/or use of antibodies or small molecule drugs targeting the gene or gene product are useful in restoring muscle mass or function in the treatment of muscle related diseases.

Low levels of expression were also seen in other metabolic tissues, including adipose and fetal heart, indicating a potential role for this gene in obesity and/or diabetes.

Moderate levels of expression were also seen in cell lines derived from brain cancer, breast cancer, renal cancer, lung cancer, colon cancer and melanoma. Since cell lines and fetal tissues are, on the whole, more proliferative than normal tissues, this expression profile indicates that this gene might be involved in cell proliferation. Therapeutic modulation of this gene, expressed protein and/or use of antibodies or small molecule drugs targeting the gene or gene product are useful in the treatment of cancer or other diseases that involve cell proliferation. Furthermore, therapeutic targeting of this gene product with a monoclonal antibody is anticipated to limit or block the extent of tumor cell migration and invasion and tumor metastasis, particularly in brain cancer, breast cancer, renal cancer, lung cancer, colon cancer and melanoma. Expression of this gene or expressed protein is useful in the diagnosis and detection of these cancers.

Panel 2D Summary: Ag2475 Highest expression of the CG58504-01 gene was seen in a lung cancer (CT=28.3). This gene encodes a putative member of the ADAMS family. The ADAMS family of proteins has multiple domains associated with function; A fibronectin domain involved cell/extracellular matrix interaction, a thrombospondin domain involved in angiogenesis and a metalloproteinase domain involved in matrix degredation. This multi-domain structure has implications for this molecule in several tumorigenic processes, including invasion and metastasis and proliferation and cell survival. Thus, the metalloproteinase domain might play a role in cell invasion and metastasis, the fibronectin domain may play a role in cell adhesion or survival and the thrombospondin domain might play a role in angiogenesis. ADAM 12-S cleaves insulin-like growth factor binding protein-3 (IGFBP-3). IGFBP-3 enhances the p53-dependent apoptotic response of colorectal cells to DNA damage. IGF-BP3 is inversely, associated with risk for colorectal cancer. Expression of IGFBP-3 induces growth inhibition and differentiation of the human colon carcinoma cell line, Caco-2. All these data indicate that the protein encoded by CG58504-01 acts by cleaving and inactivating IGFBP-3 limiting its anti-tumor activity.

Therapeutic modulation of this gene, expressed protein and/or use of antibodies or small molecule drugs targeting the gene or gene product are useful in the treatment of those cancer types, like colon, lung, kidney, bladder ovarian and gastric tumors where the gene is overexpressed in the tumor compared to the normal adjacent tissue.

Panel 4D Summary: Ag2475 Highest expression of the CG58504-01 gene was seen in resting coronary artery smooth muscle cells (CT=2,7.3). Moderate to low levels of expression were seen in resting astrocytes and TNFalpha+IL-1beta treated astrocytes and coronary artery smooth muscle cells, TNF alpha and IL-4 treated dermal fibroblasts, and lung. Lower levels of expression were seen in treated and untreated lung fibroblasts. This expression indicates that this gene is a marker of smooth muscle. In addition, expression in fibroblasts and astrocytes indicates that this gene product may be involved in inflammatory conditions that involve these cells. This gene encodes a putative ADAMTS molecule which has been implicated in extracellular proteolysis and may play a critical role in the tissue degradation seen in arthritis and other inflammatory conditions (Kuno K.: J Biol Chem 1997 Jan. 3; 272(1):556-62). Therapeutic modulation of this gene, expressed protein and/or use of antibodies or small molecule drugs targeting the gene or gene product are useful in the treatment of pathological and inflammatory lung and skin disorders that include chronic obstructive pulmonary disease, asthma, allergy, psoriasis and emphysema.

BK. CG59309-01: Acyl-Coenzyme a Thioester Hydrolase.

Expression of gene CG59309-01 was assessed using the primer-probe set Ag3540, described in Table BKA. Results of the RTQ-PCR runs are shown in Tables BKB, BKC, BKD and BKE.

TABLE BKA
Probe Name Ag3540

| | | | | SEQ |
|---|---|---|---|---|
| | | | Start | ID |
| Primers | Sequences | Length | Position | No |
| Forward | 5′-ccacgttggctctagcttatta-3′ | 22 | 649 | 1413 |
| Probe | TET-5′-tgaagatctccccaataacatggaca-3′- | 26 | 677 | 1414 |
| | TAMRA | | | |
| Reverse | 5′-ttcgaagtactccagggatatg-3′ | 22 | 704 | 1415 |

TABLE BKB
General_screening_panel_v1.4
Column A - Rel. Exp. (%) Ag3540, Run 217049291

| | |
|---|---|
| Tissue Name | A |

| | |
|---|---|
| Adipose | 1.3 |
| Melanoma* Hs688(A).T | 0.7 |
| Melanoma* Hs688(B).T | 0.5 |
| Melanoma* M14 | 0.2 |
| Melanoma* LOXIMVI | 0.0 |
| Melanoma* SK-MEL-5 | 0.0 |
| Squamous cell carcinoma SCC-4 | 0.3 |
| Testis Pool | 0.3 |
| Prostate ca.* (bone met) PC-3 | 0.8 |
| Prostate Pool | 0.3 |
| Placenta | 1.4 |
| Uterus Pool | 0.1 |
| Ovarian ca. OVCAR-3 | 1.6 |
| Ovarian ca. SK-OV-3 | 3.6 |
| Ovarian ca. OVCAR-4 | 0.4 |
| Ovarian ca. OVCAR-5 | 23.7 |
| Ovarian ca. IGROV-1 | 0.0 |
| Ovarian ca. OVCAR-8 | 0.0 |
| Ovary | 0.1 |
| Breast ca. MCF-7 | 0.0 |
| Breast ca. MDA-MB-231 | 2.5 |
| Breast ca. BT 549 | 3.0 |
| Breast ca. T47D | 100.0 |
| Breast ca. MDA-N | 0.0 |
| Breast Pool | 0.3 |
| Trachea | 0.4 |
| Lung | 0.0 |
| Fetal Lung | 0.2 |
| Lung ca. NCI-N417 | 0.0 |
| Lung ca. LX-1 | 3.5 |
| Lung ca. NCI-H146 | 0.0 |
| Lung ca. SHP-77 | 0.1 |
| Lung ca. A549 | 1.4 |
| Lung ca. NCI-H526 | 0.7 |
| Lung ca. NCI-H23 | 1.3 |
| Lung ca. NCI-H460 | 0.8 |
| Lung ca. HOP-62 | 1.2 |
| Lung ca. NCI-H522 | 0.0 |
| Liver | 2.6 |
| Fetal Liver | 0.8 |
| Liver ca. HepG2 | 0.1 |
| Kidney Pool | 0.7 |
| Fetal Kidney | 0.6 |
| Renal ca. 786-0 | 0.0 |
| Renal ca. A498 | 0.0 |
| Renal ca. ACHN | 0.0 |
| Renal ca. UO-31 | 1.1 |
| Renal ca. TK-10 | 0.1 |
| Bladder | 1.1 |
| Gastric ca. (liver met.) NCI-N87 | 5.6 |
| Gastric ca. KATO III | 0.0 |
| Colon ca. SW-948 | 0.0 |
| Colon ca. SW480 | 10.3 |
| Colon ca.* (SW480 met) SW620 | 2.8 |
| Colon ca. HT29 | 0.8 |
| Colon ca. HCT-116 | 0.0 |
| Colon ca. CaCo-2 | 3.5 |
| Colon cancer tissue | 1.4 |
| Colon ca. SW1116 | 0.0 |
| Colon ca. Colo-205 | 3.3 |
| Colon ca. SW-48 | 1.7 |
| Colon Pool | 0.2 |
| Small Intestine Pool | 0.3 |
| Stomach Pool | 0.1 |
| Bone Marrow Pool | 0.2 |
| Fetal Heart | 0.4 |
| Heart Pool | 0.2 |
| Lymph Node Pool | 0.3 |
| Fetal Skeletal Muscle | 0.1 |
| Skeletal Muscle Pool | 0.4 |
| Spleen Pool | 0.2 |
| Thymus Pool | 0.3 |
| CNS cancer (glio/astro) U87-MG | 0.0 |
| CNS cancer (glio/astro) U-118-MG | 0.3 |
| CNS cancer (neuro; met) SK-N-AS | 1.0 |
| CNS cancer (astro) SF-539 | 0.6 |
| CNS cancer (astro) SNB-75 | 3.1 |
| CNS cancer (glio) SNB-19 | 0.0 |
| CNS cancer (glio) SF-295 | 0.2 |
| Brain (Amygdala) Pool | 0.7 |
| Brain (cerebellum) | 2.1 |
| Brain (fetal) | 0.5 |
| Brain (Hippocampus) Pool | 1.0 |
| Cerebral Cortex Pool | 0.9 |
| Brain (Substantia nigra) Pool | 1.3 |
| Brain (Thalamus) Pool | 1.1 |
| Brain (whole) | 1.4 |
| Spinal Cord Pool | 0.5 |
| Adrenal Gland | 0.8 |
| Pituitary gland Pool | 0.1 |
| Salivary Gland | 0.2 |
| Thyroid (female) | 0.7 |
| Pancreatic ca. CAPAN2 | 9.4 |
| Pancreas Pool | 0.9 |

TABLE BKC
Panel 4D
Column A - Rel. Exp. (%) Ag3540, Run 166447040

| Tissue Name | A | Tissue Name | A |
|---|---|---|---|
| Secondary Th1 act | 4.8 | HUVEC IL-1 beta | 1.7 |
| Secondary Th2 act | 10.2 | HUVEC IFN gamma | 0.9 |
| Secondary Tr1 act | 12.9 | HUVEC TNF alpha + IFN gamma | 1.5 |
| Secondary Th1 rest | 2.1 | HUVEC TNF alpha + IL4 | 0.8 |
| Secondary Th2 rest | 1.4 | HUVEC IL-11 | 1.5 |
| Secondary Tr1 rest | 1.6 | Lung Microvascular EC none | 0.6 |
| Primary Th1 act | 4.7 | Lung Microvascular EC TNF alpha + IL-1 | 0.8 |
| | | beta | |
| Primary Th2 act | 6.8 | Microvascular Dermal EC none | 1.5 |
| Primary Tr1 act | 7.3 | Microsvasular Dermal EC TNF alpha + IL-1 | 0.8 |
| | | beta | |
| Primary Th1 rest | 6.6 | Bronchial epithelium TNF alpha + IL1beta | 1.3 |
| Primary Th2 rest | 2.6 | Small airway epithelium none | 0.6 |
| Primary Tr1 rest | 4.2 | Small airway epithelium TNF alpha + IL-1 | 0.0 |
| | | beta | |
| CD45RA CD4 lymphocyte act | 4.1 | Coronery artery SMC rest | 0.9 |
| CD45RO CD4 lymphocyte act | 10.9 | Coronery artery SMC TNF alpha + IL-1 beta | 0.0 |
| CD8 lymphocyte act | 6.6 | Astrocytes rest | 2.6 |
| Secondary CD8 lymphocyte rest | 17.0 | Astrocytes TNF alpha + IL-1 beta | 2.1 |
| Secondary CD8 lymphocyte act | 6.0 | KU-812 (Basophil) rest | 2.2 |
| CD4 lymphocyte none | 2.0 | KU-812 (Basophil) PMA/ionomycin | 10.2 |
| 2ry Th1/Th2/Tr1 anti-CD95 | 2.4 | CCD1106 (Keratinocytes) none | 6.8 |
| CH11 | | | |
| LAK cells rest | 2.0 | CCD1106 (Keratinocytes) TNF alpha + IL-1 | 25.7 |
| | | beta | |
| LAK cells IL-2 | 16.2 | Liver cirrhosis | 12.0 |
| LAK cells IL-2 + IL-12 | 12.8 | Lupus kidney | 5.1 |
| LAK cells IL-2 + IFN gamma | 15.6 | NCI-H292 none | 44.8 |
| LAK cells IL-2 + IL-18 | 7.4 | NCI-H292 IL-4 | 37.6 |
| LAK cells PMA/ionomycin | 3.4 | NCI-H292 IL-9 | 41.2 |
| NK Cells IL-2 rest | 9.0 | NCI-H292 IL-13 | 19.8 |
| Two Way MLR 3 day | 10.5 | NCI-H292 IFN gamma | 30.1 |
| Two Way MLR 5 day | 7.2 | HPAEC none | 1.2 |
| Two Way MLR 7 day | 8.9 | HPAEC TNF alpha + IL-1 beta | 3.3 |
| PBMC rest | 0.5 | Lung fibroblast none | 0.9 |
| PBMC PWM | 3.8 | Lung fibroblast TNF alpha + IL-1 beta | 0.7 |
| PBMC PHA-L | 1.0 | Lung fibroblast IL-4 | 0.5 |
| Ramos (B cell) none | 0.0 | Lung fibroblast IL-9 | 0.0 |
| Ramos (B cell) ionomycin | 0.0 | Lung fibroblast IL-13 | 0.9 |
| B lymphocytes PWM | 10.3 | Lung fibroblast IFN gamma | 1.2 |
| B lymphocytes CD40L and IL-4 | 3.8 | Dermal fibroblast CCD1070 rest | 1.1 |
| EOL-1 dbcAMP | 0.0 | Dermal fibroblast CCD1070 TNF alpha | 18.9 |
| EOL-1 dbcAMP | 0.0 | Dermal fibroblast CCD1070 IL-1 beta | 1.9 |
| PMA/ionomycin | | | |
| Dendritic cells none | 14.9 | Dermal fibroblast IFN gamma | 0.0 |
| Dendritic cells LPS | 8.9 | Dermal fibroblast IL-4 | 1.5 |
| Dendritic cells anti-CD40 | 7.9 | IBD Colitis 2 | 2.9 |
| Monocytes rest | 0.0 | IBD Crohn's | 1.9 |
| Monocytes LPS | 0.6 | Colon | 82.9 |
| Macrophages rest | 40.3 | Lung | 9.7 |
| Macrophages LPS | 6.1 | Thymus | 100.0 |
| HUVEC none | 1.1 | Kidney | 1.8 |
| HUVEC starved | 1.4 | | |

TABLE BKD
Panel 5 Islet
Column A - Rel. Exp. (%) Ag3540, Run 242386396

| Tissue Name | A | Tissue Name | A |
|---|---|---|---|
| 97457 Patient-02go adipose | 3.3 | 94709 Donor 2 AM - A adipose | 9.1 |
| 97476 Patient-07sk skeletal muscle | 0.8 | 94710 Donor 2 AM - B adipose | 1.6 |
| 97477 Patient-07ut uterus | 0.0 | 94711 Donor 2 AM - C adipose | 1.4 |
| 97478 Patient-07pl placenta | 12.9 | 94712 Donor 2 AD - A adipose | 2.8 |
| 99167 Bayer Patient 1 | 15.5 | 94713 Donor 2 AD - B adipose | 5.8 |
| 97482 Patient-08ut uterus | 3.4 | 94714 Donor 2 AD - C adipose | 4.2 |
| 97483 Patient-08pl placenta | 3.4 | 94742 Donor 3 U - A Mesenchymal | 3.0 |
| | | Stem Cells | |
| 97486 Patient-09sk skeletal muscle | 100.0 | 94743 Donor 3 U - B Mesenchymal | 1.1 |
| | | Stem Cells | |
| 97487 Patient-09ut uterus | 1.6 | 94730 Donor 3 AM - A adipose | 4.3 |
| 97488 Patient-09pl placenta | 2.6 | 94731 Donor 3 AM - B adipose | 2.0 |
| 97492 Patient-10ut uterus | 3.1 | 94732 Donor 3 AM - C adipose | 2.0 |
| 97493 Patient-10pl placenta | 23.2 | 94733 Donor 3 AD - A adipose | 10.7 |
| 97495 Patient-11go adipose | 0.8 | 94734 Donor 3 AD - B adipose | 3.0 |
| 97496 Patient-11sk skeletal muscle | 0.0 | 94735 Donor 3 AD - C adipose | 4.0 |
| 97497 Patient-11ut uterus | 2.5 | 77138 Liver HepG2untreated | 0.7 |
| 97498 Patient-11pl placenta | 6.7 | 73556 Heart Cardiac stromal cells | 0.0 |
| | | (primary) | |
| 97500 Patient-12go adipose | 6.5 | 81735 Small Intestine | 4.8 |
| 97501 Patient-12sk skeletal muscle | 4.5 | 72409 Kidney Proximal Convoluted | 0.7 |
| | | Tubule | |
| 97502 Patient-12ut uterus | 6.7 | 82685 Small intestine Duodenum | 3.6 |
| 97503 Patient-12p1 placenta | 2.4 | 90650 Adrenal Adrenocortical | 0.6 |
| | | adenoma | |
| 94721 Donor 2 U - A Mesenchymal | 2.2 | 72410 Kidney HRCE | 8.0 |
| Stem Cells | | | |
| 94722 Donor 2 U - B Mesenchymal | 0.6 | 72411 Kidney HRE | 8.5 |
| Stem Cells | | | |
| 94723 Donor 2 U - C Mesenchymal | 3.1 | 73139 Uterus Uterine smooth muscle | 0.0 |
| Stem Cells | | cells | |

TABLE BKE
general oncology screening panel_v_2.4
Column A - Rel. Exp. (%) Ag3540, Run 267294323

| Tissue Name | A | Tissue Name | A |
|---|---|---|---|
| Colon cancer 1 | 2.8 | Bladder NAT 2 | 0.0 |
| CC Margin (ODO3921) | 2.9 | Bladder NAT 3 | 0.0 |
| Colon cancer 2 | 6.9 | Bladder NAT 4 | 0.0 |
| Colon NAT 2 | 3.3 | Prostate adenocarcinoma 1 | 3.5 |
| Colon cancer 3 | 7.9 | Prostate adenocarcinoma 2 | 0.0 |
| Colon NAT 3 | 5.0 | Prostate adenocarcinoma 3 | 1.1 |
| Colon malignant cancer 4 | 7.1 | Prostate adenocarcinoma 4 | 0.8 |
| Colon NAT 4 | 0.9 | Prostate NAT 5 | 0.4 |
| Lung cancer 1 | 1.9 | Prostate adenocarcinoma 6 | 0.6 |
| Lung NAT 1 | 0.0 | Prostate adenocarcinoma 7 | 0.0 |
| Lung cancer 2 | 0.0 | Prostate adenocarcinoma 8 | 0.0 |
| Lung NAT 2 | 0.0 | Prostate adenocarcinoma 9 | 0.8 |
| Squamous cell carcinoma 3 | 2.6 | Prostate NAT 10 | 0.0 |
| Lung NAT 3 | 0.0 | Kidney cancer 1 | 5.1 |
| Metastatic melanoma 1 | 2.4 | Kidney NAT 1 | 2.3 |
| Melanoma 2 | 0.4 | Kidney cancer 2 | 100.0 |
| Melanoma 3 | 0.0 | Kidney NAT 2 | 5.1 |
| Metastatic melanoma 4 | 2.7 | Kidney cancer 3 | 7.7 |
| Metastatic melanoma 5 | 6.9 | Kidney NAT 3 | 1.3 |
| Bladder cancer 1 | 0.0 | Kidney cancer 4 | 1.3 |
| Bladder NAT 1 | 0.0 | Kidney NAT 4 | 13.1 |
| Bladder cancer 2 | 0.4 | | |

General_screening_panel_v1.4 Summary: Ag3540 This gene was most highly expressed in a breast cancer cell line (CT=27.1). Expression of this gene is useful as a marker to detect the presence of breast cancer. Therapeutic modulation of this gene, expressed protein and/or use of antibodies or small molecule drugs targeting the gene or gene product are useful in the treatment of breast cancer.

Among metabolic tissues, this gene, an acyl coA thioesterase homolog, had a low level of expression in adipose, adult and fetal liver, adrenal, thyroid and pancreas. Acyl CoA thioesterases have multiple roles in lipid homeostasis (Hunt M C, Alexson S E. The role Acyl-CoA thioesterases play in mediating intracellular lipid metabolism. Prog Lipid Res. 2002 March; 41(2):99-130; Hunt M C, Nousiainen S E, Huttunen M K, Orii K E, Svensson L T, Alexson S E. Peroxisome proliferator-induced long chain acyl-CoA thioesterases comprise a highly conserved novel multi-gene family involved in lipid metabolism. J Biol. Chem. 1999 Nov. 26; 274(48):34317-26). Therapeutic modulation of this gene, expressed protein and/or use of antibodies or small molecule drugs targeting the gene or gene product are useful in the treatment of endocrine and metabolic disease, including Types 1 and 2 diabetes and obesity.

In addition, this gene was expressed in all CNS regions examined. Therapeutic modulation of this gene, expressed protein and/or use of antibodies or small molecule drugs targeting the gene or gene product are useful in the treatment of neurologic disorders such as Alzheimer's disease, Parkinson's disease, epilepsy, stroke, schizophrenia and multiple sclerosis.

Panel 4D Summary: Ag3540 Highest expression of the CG59309-01 gene was seen in the thymus and colon (CTs=31.5). Significant levels of expression were also seen in a cluster of treated and untreated samples derived from the NCI-H292 mucoepidermoid cell line. Expression of this gene is useful as a marker for thymus and colon tissue. Therapeutic modulation of this gene, expressed protein and/or use of antibodies or small molecule drugs targeting the gene or gene product are useful in regulating T cell development in the thymus or in the treatment of T cell mediated autoimmune or inflammatory diseases, including asthma, allergies, inflammatory bowel disease, lupus erythematosus, or rheumatoid arthritis. Small molecule or antibody therapeutics designed against this protein disrupts T cell development in the thymus and functions as an immunosuppresant for tissue transplants.

Panel 5 Islet Summary: Ag3540 This gene had moderate expression in skeletal muscle (highest expression CT=30.5). Acyl CoA thioesterases function in peroxisomal fatty acid oxidation (Hunt M C, Solaas K, Kase B F, Alexson S E. Characterization of an acyl-coA thioesterase that functions as a major regulator of peroxisomal lipid metabolism. J Biol. Chem. 2002 Jan. 11; 277(2): 1128-38). Therapeutic modulation of this gene, expressed protein and/or use of antibodies or small molecule drugs targeting the gene or gene product are useful in increasing fatty acid oxidation in muscle, and in the treatment of Type 2 diabetes and obesity.

general oncology screening panel_v_{—}2.4 Summary: Ag3540 Prominent expression was detected in a kidney cancer sample (CT=31.8). Expression of this gene is useful as a marker of this cancer. Targeting this gene or gene product with small molecule, antibody, or protein therapeutics is useful in the treatment of kidney cancer.

BL. CG59490-01: S562_F7.

Expression of gene CG59490-01 was assessed using the primer-probe sets Ag1038, Ag1590, Ag1918, Ag2899, Ag720, Ag730 and Ag443, described in Tables BLA, BLB, BLC, BLD, BLE, BLF and BLG. Results of the RTQ-PCR runs are shown in Tables BLH, BLI, BLJ, BLK and BLL.

TABLE BLA
Probe Name Ag1038

| | | | | SEQ |
|---|---|---|---|---|
| | | | Start | ID |
| Primers | Sequences | Length | Position | No |
| Forward | 5′-aggagcaacgtcctctgtaac-3′ | 21 | 367 | 1416 |
| Probe | TET-5′-cttccaaccacactgagcggtttgag-3′- | 26 | 410 | 1417 |
| | TAMRA | | | |
| Reverse | 5-agcatgtcgtccttgatgag-3′ | 20 | 439 | 1418 |

TABLE BLB
Probe Name Ag1590

| | | | | SEQ |
|---|---|---|---|---|
| | | | Start | ID |
| Primers | Sequences | Length | Position | No |
| Forward | 5′-aggagcaacgtcctctgtaac-3′ | 21 | 367 | 1419 |
| Probe | TET-5′-cttccaaccacactgagcggtttgag-3′- | 26 | 410 | 1420 |
| | TAMRA | | | |
| Reverse | 5′-agcatgtcgtccttgatgag-3′ | 20 | 439 | 1421 |

TABLE BLC
Probe Name Ag1918

| | | | | SEQ |
|---|---|---|---|---|
| | | | Start | ID |
| Primers | Sequences | Length | Position | No |
| Forward | 5′-aggagcaacgtcctctgtaac-3′ | 21 | 367 | 1422 |
| Probe | TET-5′-cttccaaccacactgagcggtttgag-3′- | 26 | 410 | 1423 |
| | TAMRA | | | |
| Reverse | 5′-agcatgtcgtccttgatgag-3′ | 20 | 439 | 1424 |

TABLE BLD
Probe Name Ag2899

| | | | | SEQ |
|---|---|---|---|---|
| | | | Start | ID |
| Primers | Sequences | Length | Position | No |
| Forward | 5′-aggagcaacgtcctctgtaac-3′ | 21 | 367 | 1425 |
| Probe | TET-5′-cttccaaccacactgagcggtttgag-3′ | 26 | 410 | 1426 |
| | TAMRA | | | |
| Reverse | 5′-agcatgtcgtccttgatgag-3′ | 20 | 439 | 1427 |

TABLE BLE
Probe Name Ag720

| | | | | SEQ |
|---|---|---|---|---|
| | | | Start | ID |
| Primers | Sequences | Length | Position | No |
| Forward | 5′-aggagcaacgtcctctgtaac-3′ | 21 | 367 | 1428 |
| Probe | TET-5′-cttccaaccacactgagcggtttgag-3′- | 26 | 410 | 1429 |
| | TAMRA | | | |
| Reverse | 5′-acagcatgtcgtccttgatg-3′ | 20 | 441 | 1430 |

TABLE BLF
Probe Name Ag730

| | | | | SEQ |
|---|---|---|---|---|
| | | | Start | ID |
| Primers | Sequences | Length | Position | No |
| Forward | 5′-caggagcaacgtcctctgta-3′ | 20 | 366 | 1431 |
| Probe | TET-5′-cttccaaccacactgagcggtttgag-3′- | 26 | 410 | 1432 |
| | TAMRA | | | |
| Reverse | 5′-cacacagcatgtcgtcctt-3′ | 19 | 445 | 1433 |

TABLE BLG
Probe Name Ag443

| | | | | SEQ |
|---|---|---|---|---|
| | | | Start | ID |
| Primers | Sequences | Length | Position | No |
| Forward | 5′-gacggtgaaggtcaggagca-3′ | 20 | 354 | 1434 |
| Probe | TET-5′-cctgtcgccgccgctttcc-3′-TAMRA | 19 | 392 | 1435 |
| Reverse | 5′-aaccgctcagtgtggttgg-3′ | 19 | 413 | 1436 |

TABLE BLH
Ardais Breast1.0
Column A - Rel. Exp. (%) Ag720, Run 389241931

| Tissue Name | A |
|---|---|
| 111297 Breast cancer metastasis (9369)* | 0.0 |
| 108830 Breast cancer metastasis (OD06855)* | 1.1 |
| 97764 Breast cancer node metastasis (OD06083) | 1.3 |
| 97739 Breast cancer (CHTN20676) | 0.1 |
| 145848 Breast cancer (9B6) | 8.4 |
| 145859 Breast cancer (9EC) | 1.2 |
| 153632 Breast cancer (D39) | 0.0 |
| 153636 Breast cancer (D3D) | 0.4 |
| 164668 Breast cancer (6314) | 0.1 |
| 164677 Breast cancer (5272) | 12.4 |
| 164685 Breast cancer (0170) | 11.3 |
| 98857 Breast cancer (OD06397-12) | 0.0 |
| 153628 Breast cancer (D35) | 0.8 |
| 153637 Breast cancer (D3E) | 5.4 |
| 153643 Breast cancer (D44) | 10.7 |
| 164672 Breast cancer (7464) | 26.1 |
| 164681 Breast cancer (5787) | 9.7 |
| 97748 Breast cancer (CHTN20931) | 2.2 |
| 145850 Breast cancer (9C7) | 0.1 |
| 149844 Breast cancer (24178) | 2.5 |
| 153633 Breast cancer (D3A) | 0.4 |
| 153644 Breast cancer (D45) | 9.0 |
| 164673 Breast cancer (8452) | 29.1 |
| 164682 Breast cancer (6342)* | 7.7 |
| 97751 Breast cancer (CHTN21053) | 0.8 |
| 116417 Breast cancer (3367)* | 0.1 |
| 145852 Breast cancer (A1A) | 13.0 |
| 151097 Breast cancer (CHTN24298) | 0.4 |
| 153634 Breast cancer (D3B) | 0.8 |
| 155797 Breast cancer (EA6) | 2.2 |
| 164674 Breast cancer (8811) | 8.2 |
| 164683 Breast cancer (6470) | 2.4 |
| 97763 Breast cancer (OD06083) | 0.8 |
| 116418 Breast cancer (3378)* | 1.3 |
| 145853 Breast cancer (9F3) | 0.4 |
| 153432 Breast cancer (CHTN 24652) | 16.6 |
| 153635 Breast cancer (D3C) | 1.1 |
| 164667 Breast cancer (5785) | 1.6 |
| 164676 Breast cancer (5070) | 100.0 |
| 164684 Breast cancer (6509) | 24.8 |
| 116421 Breast cancer (6314) | 0.0 |
| 145854 Breast cancer (9B8) | 5.9 |
| 153627 Breast cancer (D34) | 0.2 |
| 164669 Breast cancer (6992) | 6.0 |
| 164678 Breast cancer (5297) | 68.8 |
| 164686 Breast cancer (0732) | 0.5 |
| 145857 Breast cancer (9F0) | 15.2 |
| 153630 Breast cancer (D37) | 0.6 |
| 153638 Breast cancer (D3F) | 23.7 |
| 164670 Breast cancer (7078) | 1.1 |
| 164679 Breast cancer (5486) | 4.3 |
| 164687 Breast cancer (5881) | 0.5 |
| 145846 Breast cancer (9B7) | 22.5 |
| 145858 Breast cancer (9B4) | 0.0 |
| 153631 Breast cancer (D38) | 1.6 |
| 153639 Breast cancer (D40) | 22.2 |
| 164671 Breast cancer (7082) | 0.3 |
| 164680 Breast cancer (5705) | 0.0 |
| 164688 Breast cancer (7222) | 0.0 |
| 111288 Breast NAT (3367) | 0.3 |
| 111302 Breast NAT (6314) | 0.6 |
| 105687 Breast cancer 1B | 0.0 |
| 105688 Breast NAT 1A | 0.0 |
| 105689 Breast cancer 2B | 0.0 |
| 105690 Breast NAT 2A | 0.0 |
| 111289 Breast cancer 3B* | 0.0 |
| 111290 Breast NAT 3A* | 0.0 |
| 116424 Breast cancer 4B* | 0.1 |
| 116425 Breast NAT 4A | 0.0 |
| 108847 Breast cancer | 0.0 |
| 105694 Breast NAT | 0.0 |

TABLE BLI
Panel 1.3D
Column A - Rel. Exp. (%) Ag1590, Run 152059684
Column B - Rel. Exp. (%) Ag1590, Run 155330156
Column C - Rel. Exp. (%) Ag2899, Run 160943164
Column D - Rel. Exp. (%) Ag2899, Run 165518181

| Tissue Name | A | B | C | D |
|---|---|---|---|---|
| Liver adenocarcinoma | 0.0 | 1.1 | 1.7 | 0.0 |
| Pancreas | 0.0 | 0.0 | 0.0 | 6.9 |
| Pancreatic ca. CAPAN 2 | 8.7 | 2.5 | 5.6 | 10.5 |
| Adrenal gland | 0.0 | 0.8 | 0.0 | 0.0 |
| Thyroid | 0.0 | 0.0 | 0.0 | 0.0 |
| Salivary gland | 0.0 | 0.0 | 0.0 | 2.4 |
| Pituitaiy gland | 34.2 | 34.2 | 16.7 | 42.0 |
| Brain (fetal) | 0.0 | 0.0 | 0.0 | 1.9 |
| Brain (whole) | 0.0 | 0.0 | 0.0 | 0.0 |
| Brain (amygdala) | 0.0 | 0.0 | 0.0 | 0.0 |
| Brain (cerebellum) | 0.0 | 0.0 | 0.0 | 0.0 |
| Brain (hippocampus) | 0.0 | 0.0 | 0.0 | 0.0 |
| Brain (substantia nigra) | 0.0 | 0.0 | 0.0 | 0.0 |
| Brain (thalamus) | 0.0 | 0.0 | 0.0 | 0.0 |
| Cerebral Cortex | 1.2 | 0.0 | 0.0 | 0.0 |
| Spinal cord | 0.0 | 0.0 | 0.0 | 0.0 |
| glio/astro U87-MG | 0.0 | 0.0 | 0.0 | 0.0 |
| glio/astro U-118-MG | 0.0 | 0.0 | 0.0 | 0.0 |
| astrocytoma SW1783 | 0.3 | 1.0 | 0.0 | 2.3 |
| neuro*; met SK-N-AS | 0.0 | 0.0 | 0.0 | 0.0 |
| astrocytoma SF-539 | 0.0 | 0.0 | 0.0 | 0.0 |
| astrocytoma SNB-75 | 0.7 | 0.0 | 0.0 | 4.5 |
| glioma SNB-19 | 0.0 | 0.0 | 0.0 | 2.5 |
| glioma U251 | 0.0 | 0.0 | 0.0 | 0.0 |
| glioma SF-295 | 0.0 | 0.0 | 0.0 | 0.0 |
| Heart (Fetal) | 0.0 | 1.4 | 0.0 | 0.0 |
| Heart | 0.0 | 0.0 | 0.0 | 0.0 |
| Skeletal muscle (Fetal) | 0.0 | 0.0 | 0.0 | 0.0 |
| Skeletal muscle | 0.0 | 0.0 | 0.0 | 0.0 |
| Bone marrow | 0.9 | 0.0 | 1.0 | 0.0 |
| Thymus | 0.7 | 0.9 | 1.4 | 3.7 |
| Spleen | 0.6 | 1.7 | 0.0 | 0.0 |
| Lymph node | 0.0 | 0.0 | 0.0 | 0.0 |
| Colorectal | 3.9 | 2.4 | 1.6 | 8.8 |
| Stomach | 0.0 | 2.4 | 0.0 | 0.0 |
| Small intestine | 0.0 | 0.0 | 0.0 | 2.0 |
| Colon ca. SW480 | 0.8 | 1.5 | 0.0 | 0.0 |
| Colon ca.* SW620 (SW480 met) | 0.9 | 0.9 | 3.4 | 0.0 |
| Colon ca. HT29 | 0.0 | 0.0 | 0.0 | 0.0 |
| Colon ca. HCT-116 | 0.0 | 1.0 | 0.0 | 1.0 |
| Colon ca. CaCo-2 | 0.0 | 1.4 | 0.0 | 0.0 |
| CC Well to Mod Diff (ODO3866) | 8.8 | 8.1 | 15.2 | 26.8 |
| Colon ca. HCC-2998 | 32.3 | 29.3 | 29.9 | 22.2 |
| Gastric ca. (liver met) NCI-N87 | 32.1 | 31.6 | 36.9 | 70.7 |
| Bladder | 0.8 | 0.0 | 0.0 | 0.0 |
| Trachea | 0.8 | 1.1 | 0.0 | 0.0 |
| Kidney | 1.8 | 0.0 | 1.7 | 1.0 |
| Kidney (fetal) | 3.8 | 0.9 | 7.6 | 0.0 |
| Renal ca. 786-0 | 0.0 | 0.0 | 0.0 | 0.0 |
| Renal ca. A498 | 0.8 | 0.0 | 0.0 | 0.0 |
| Renal ca. RXF 393 | 0.0 | 0.0 | 0.0 | 3.2 |
| Renal ca. ACHN | 2.1 | 0.0 | 0.0 | 0.0 |
| Renal ca. UO-31 | 0.0 | 0.0 | 0.0 | 0.0 |
| Renal ca. TK-10 | 0.0 | 0.0 | 0.0 | 0.0 |
| Liver | 4.7 | 5.2 | 3.5 | 33.9 |
| Liver (fetal) | 9.0 | 6.0 | 3.7 | 18.3 |
| Liver ca. (hepatoblast) HepG2 | 2.4 | 1.2 | 1.8 | 0.0 |
| Lung | 0.0 | 0.0 | 3.2 | 0.0 |
| Lung (fetal) | 0.4 | 0.0 | 0.0 | 0.0 |
| Lung ca. (small cell) LX-1 | 6.9 | 13.1 | 9.5 | 10.5 |
| Lung ca. (small cell) NCI-H69 | 2.2 | 1.3 | 0.8 | 0.0 |
| Lung ca. (s. cell var.) SHP-77 | 3.3 | 1.1 | 6.5 | 6.3 |
| Lung ca. (large cell) NCI-H460 | 0.0 | 0.0 | 0.0 | 0.0 |
| Lung ca. (non-sm. cell) A549 | 0.0 | 0.0 | 0.0 | 0.0 |
| Lung ca. (non-s.cell) NCI-H23 | 0.0 | 0.0 | 0.0 | 0.0 |
| Lung ca. (non-s.cell) HOP-62 | 0.8 | 0.0 | 0.0 | 0.0 |
| Lung ca. (non-s.cl) NCI-H522 | 0.0 | 0.5 | 0.0 | 0.0 |
| Lung ca. (squam.) SW 900 | 0.8 | 0.0 | 0.0 | 0.0 |
| Lung ca. (squam.) NCI-H596 | 0.4 | 0.5 | 0.0 | 0.0 |
| Mammary gland | 2.7 | 4.2 | 0.0 | 7.9 |
| Breast ca.* (pl.ef) MCF-7 | 4.1 | 0.0 | 0.0 | 2.5 |
| Breast ca.* (pl.ef) MDA-MB-231 | 0.0 | 0.0 | 1.7 | 0.0 |
| Breast ca.* (p1. ef) T47D | 6.5 | 5.3 | 0.7 | 4.3 |
| Breast ca. BT-549 | 0.0 | 0.0 | 0.0 | 0.0 |
| Breast ca. MDA-N | 0.0 | 1.1 | 1.7 | 0.0 |
| Ovary | 0.0 | 0.0 | 0.0 | 0.0 |
| Ovarian ca. OVCAR-3 | 0.0 | 0.0 | 0.0 | 0.0 |
| Ovarian ca. OVCAR-4 | 0.0 | 0.0 | 0.0 | 0.0 |
| Ovarian ca. OVCAR-5 | 100.0 | 100.0 | 100.0 | 100.0 |
| Ovarian ca. OVCAR-8 | 2.5 | 0.0 | 0.0 | 2.5 |
| Ovarian ca. IGROV-1 | 0.0 | 0.0 | 0.0 | 0.0 |
| Ovarian ca. (ascites) SK-OV-3 | 0.0 | 0.0 | 0.0 | 0.0 |
| Uterus | 0.0 | 0.0 | 0.0 | 0.0 |
| Placenta | 9.2 | 6.9 | 8.0 | 14.0 |
| Prostate | 1.9 | 5.2 | 1.8 | 4.1 |
| Prostate ca.* (bone met) PC-3 | 0.0 | 0.0 | 0.0 | 0.0 |
| Testis | 6.3 | 9.3 | 8.9 | 8.8 |
| Melanoma Hs688(A).T | 0.0 | 0.0 | 0.0 | 0.0 |
| Melanoma* (met) Hs688(B).T | 0.0 | 0.0 | 0.0 | 0.0 |
| Melanoma UACC-62 | 0.0 | 0.0 | 0.0 | 0.0 |
| Melanoma M14 | 0.0 | 0.0 | 0.0 | 0.0 |
| Melanoma LOX IMVI | 0.0 | 0.0 | 0.0 | 0.0 |
| Melanoma* (met) SK-MEL-5 | 0.0 | 0.0 | 0.0 | 0.0 |
| Adipose | 0.0 | 0.0 | 0.0 | 0.0 |

TABLE BLJ
Panel 2D
Column A - Rel. Exp. (%) Ag1590, Run 152060853
Column B - Rel. Exp. (%) Ag1590, Run 155330182
Column C - Rel. Exp. (%) Ag2899, Run 160999416
Column D - Rel. Exp. (%) Ag2899, Run 164988403
Column E - Rel. Exp. (%) Ag720, Run 145375720

| Tissue Name | A | B | C | D | E |
|---|---|---|---|---|---|
| Normal Colon | 0.1 | 0.0 | 0.1 | 0.3 | 0.0 |
| CC Well to Mod Diff (ODO3866) | 9.2 | 11.3 | 5.5 | 8.5 | 2.2 |
| CC Margin (ODO3866) | 0.2 | 0.0 | 0.0 | 0.3 | 0.0 |
| CC Gr. 2 rectosigmoid (ODO3868) | 0.0 | 0.2 | 0.0 | 0.3 | 0.0 |
| CC Margin (ODO3868) | 0.0 | 0.0 | 0.0 | 0.0 | 0.0 |
| CC Mod Diff (ODO3920) | 0.2 | 0.1 | 0.0 | 0.5 | 0.0 |
| CC Margin (ODO3920) | 0.0 | 0.0 | 0.0 | 0.0 | 0.0 |
| CC Gr. 2 ascend colon (ODO3921) | 0.0 | 0.0 | 0.1 | 0.1 | 0.0 |
| CC Margin (ODO3921) | 0.3 | 0.2 | 0.1 | 0.1 | 0.0 |
| CC from Partial Hepatectomy (ODO4309) Mets | 1.4 | 1.4 | 0.2 | 1.8 | 0.3 |
| Liver Margin (ODO4309) | 1.8 | 0.9 | 0.6 | 2.0 | 0.4 |
| Colon mets to lung (ODO4451-01) | 0.2 | 0.2 | 0.3 | 0.4 | 0.2 |
| Lung Margin (OD04451-02) | 0.0 | 0.0 | 0.0 | 0.0 | 0.0 |
| Normal Prostate 6546-1 | 0.4 | 0.7 | 0.2 | 1.6 | 0.4 |
| Prostate Cancer (OD04410) | 0.0 | 0.0 | 0.0 | 0.0 | 0.0 |
| Prostate Margin (OD04410) | 0.0 | 0.0 | 0.0 | 0.4 | 0.0 |
| Prostate Cancer (OD04720-01) | 0.9 | 1.2 | 0.3 | 0.5 | 0.4 |
| Prostate Margin (OD04720-02) | 0.3 | 0.4 | 0.3 | 0.0 | 0.1 |
| Normal Lung | 0.0 | 0.0 | 0.1 | 0.0 | 0.0 |
| Lung Met to Muscle (ODO4286) | 0.0 | 0.3 | 0.0 | 0.0 | 0.2 |
| Muscle Margin (ODO4286) | 0.2 | 0.0 | 0.0 | 0.1 | 0.0 |
| Lung Malignant Cancer (OD03126) | 0.0 | 0.3 | 0.0 | 0.2 | 0.2 |
| Lung Margin (OD03126) | 0.0 | 0.1 | 0.0 | 0.0 | 0.0 |
| Lung Cancer (OD04404) | 0.0 | 0.0 | 0.0 | 0.0 | 0.1 |
| Lung Margin (OD04404) | 0.0 | 0.0 | 0.0 | 0.0 | 0.0 |
| Lung Cancer (OD04565) | 0.2 | 0.0 | 0.0 | 0.4 | 0.1 |
| Lung Margin (OD04565) | 0.0 | 0.0 | 0.0 | 0.0 | 0.0 |
| Lung Cancer (OD04237-01) | 0.0 | 0.2 | 0.0 | 0.5 | 0.0 |
| Lung Margin (OD04237-02) | 0.0 | 0.0 | 0.0 | 0.0 | 0.0 |
| Ocular Mel Met to Liver (ODO4310) | 0.0 | 0.0 | 0.0 | 0.0 | 0.0 |
| Liver Margin (ODO4310) | 1.0 | 1.1 | 0.0 | 1.0 | 0.5 |
| Melanoma Metastasis | 0.0 | 0.0 | 0.0 | 0.0 | 0.0 |
| Lung Margin (OD04321) | 0.0 | 0.0 | 0.0 | 0.0 | 0.0 |
| Normal Kidney | 0.3 | 1.1 | 0.1 | 0.7 | 0.3 |
| Kidney Ca, Nuclear grade 2 (OD04338) | 0.0 | 0.0 | 0.1 | 0.1 | 0.0 |
| Kidney Margin (OD04338) | 0.2 | 0.7 | 0.2 | 0.2 | 0.1 |
| Kidney Ca Nuclear grade 1/2 (OD04339) | 0.1 | 0.1 | 0.3 | 0.4 | 0.0 |
| Kidney Margin (OD04339) | 0.6 | 0.3 | 0.7 | 1.4 | 0.3 |
| Kidney Ca, Clear cell type (OD04340) | 0.0 | 0.0 | 0.0 | 0.0 | 0.0 |
| Kidney Margin (OD04340) | 1.2 | 0.8 | 0.4 | 0.9 | 0.5 |
| Kidney Ca, Nuclear grade 3 (OD04348) | 0.0 | 0.0 | 0.0 | 0.6 | 0.0 |
| Kidney Margin (OD04348) | 0.4 | 0.4 | 0.4 | 0.0 | 0.3 |
| Kidney Cancer (OD04622-01) | 0.0 | 0.0 | 0.0 | 0.0 | 0.0 |
| Kidney Margin (OD04622-03) | 0.4 | 0.1 | 0.0 | 0.0 | 0.2 |
| Kidney Cancer (OD04450-01) | 0.3 | 0.2 | 0.1 | 0.0 | 0.0 |
| Kidney Margin (OD04450-03) | 0.0 | 0.2 | 0.0 | 0.3 | 0.2 |
| Kidney Cancer 8120607 | 0.0 | 0.0 | 0.0 | 0.0 | 0.0 |
| Kidney Margin 8120608 | 0.0 | 0.4 | 0.1 | 0.0 | 0.1 |
| Kidney Cancer 8120613 | 0.1 | 0.9 | 0.3 | 0.6 | 0.2 |
| Kidney Margin 8120614 | 0.3 | 0.3 | 0.0 | 0.0 | 0.0 |
| Kidney Cancer 9010320 | 0.0 | 0.1 | 0.0 | 0.0 | 0.0 |
| Kidney Margin 9010321 | 0.1 | 0.0 | 0.0 | 0.0 | 0.1 |
| Normal Uterus | 0.0 | 0.0 | 0.0 | 0.0 | 0.0 |
| Uterine Cancer 064011 | 0.0 | 0.0 | 0.0 | 0.0 | 0.0 |
| Normal Thyroid | 0.0 | 0.1 | 0.0 | 0.0 | 0.0 |
| Thyroid Cancer | 0.0 | 0.3 | 0.3 | 0.0 | 0.0 |
| Thyroid Cancer A302152 | 0.1 | 0.0 | 0.0 | 0.0 | 0.1 |
| Thyroid Margin A302153 | 0.0 | 0.0 | 0.0 | 0.0 | 0.0 |
| Normal Breast | 0.3 | 0.4 | 0.1 | 1.2 | 0.5 |
| Breast Cancer | 33.4 | 34.6 | 22.7 | 55.9 | 47.0 |
| Breast Cancer (OD04590-01) | 100.0 | 100.0 | 100.0 | 100.0 | 30.6 |
| Breast Cancer Mets (OD04590-03) | 63.3 | 72.7 | 73.7 | 88.9 | 100.0 |
| Breast Cancer Metastasis | 2.8 | 2.6 | 2.0 | 2.6 | 2.9 |
| Breast Cancer | 3.6 | 5.2 | 2.2 | 6.4 | 3.1 |
| Breast Cancer | 1.0 | 1.9 | 2.2 | 1.9 | 0.5 |
| Breast Cancer 9100266 | 3.5 | 3.3 | 1.1 | 7.1 | 4.1 |
| Breast Margin 9100265 | 0.0 | 0.3 | 0.1 | 0.2 | 0.3 |
| Breast Cancer A209073 | 2.3 | 1.6 | 0.7 | 2.0 | 1.7 |
| Breast Margin A209073 | 0.7 | 1.3 | 0.7 | 0.9 | 0.2 |
| Normal Liver | 0.1 | 0.5 | 0.4 | 2.2 | 0.9 |
| Liver Cancer | 0.3 | 0.3 | 0.4 | 0.3 | 0.1 |
| Liver Cancer 1025 | 0.4 | 0.7 | 0.5 | 1.3 | 0.5 |
| Liver Cancer 1026 | 0.7 | 0.1 | 0.3 | 1.1 | 0.2 |
| Liver Cancer 6004-T | 0.9 | 1.1 | 1.4 | 2.8 | 0.6 |
| Liver Tissue 6004-N | 0.0 | 0.0 | 0.1 | 0.0 | 0.0 |
| Liver Cancer 6005-T | 0.8 | 0.8 | 1.2 | 0.7 | 0.2 |
| Liver Tissue 6005-N | 0.1 | 0.0 | 0.2 | 0.5 | 0.0 |
| Normal Bladder | 0.0 | 0.0 | 0.1 | 0.4 | 0.1 |
| Bladder Cancer | 0.0 | 0.1 | 0.0 | 0.0 | 0.1 |
| Bladder Cancer | 0.1 | 0.4 | 0.3 | 0.0 | 0.1 |
| Bladder Cancer (OD04718-01) | 0.2 | 0.2 | 0.3 | 0.0 | 0.4 |
| Bladder Normal Adjacent (OD04718-03) | 0.0 | 0.0 | 0.0 | 0.0 | 0.0 |
| Normal Ovary | 0.0 | 0.0 | 0.0 | 0.0 | 0.0 |
| Ovarian Cancer | 0.0 | 0.0 | 0.0 | 0.2 | 0.0 |
| Ovarian Cancer (OD04768-07) | 0.1 | 0.0 | 0.1 | 0.0 | 0.0 |
| Ovary Margin (OD04768-08) | 0.0 | 0.0 | 0.0 | 0.0 | 0.0 |
| Normal Stomach | 0.0 | 0.1 | 0.0 | 0.2 | 0.0 |
| Gastric Cancer 9060358 | 0.0 | 0.0 | 0.0 | 0.0 | 0.0 |
| Stomach Margin 9060359 | 0.0 | 0.0 | 0.0 | 0.0 | 0.0 |
| Gastric Cancer 9060395 | 0.0 | 0.0 | 0.0 | 0.0 | 0.0 |
| Stomach Margin 9060394 | 0.0 | 0.0 | 0.0 | 0.0 | 0.0 |
| Gastric Cancer 9060397 | 1.5 | 0.7 | 0.6 | 0.3 | 0.2 |
| Stomach Margin 9060396 | 0.0 | 0.0 | 0.0 | 0.0 | 0.0 |
| Gastric Cancer 064005 | 0.1 | 0.3 | 0.0 | 0.2 | 0.2 |

TABLE BLK
Panel 3D
Column A - Rel. Exp. (%) Ag2899, Run 164633619
Column B - Rel. Exp. (%) Ag720, Run 164843791

| Tissue Name | A | B | Tissue Name | A | B |
|---|---|---|---|---|---|
| 94905 Daoy | 0.0 | 0.0 | 94954 Ca Ski Cervical | 0.0 | 3.2 |
| Medulloblastoma/Cerebellum | | | epidermoid carcinoma | | |
| | | | (metastasis | | |
| 94906 TE671 | 0.0 | 0.0 | 94955 ES-2 Ovarian clear cell | 0.0 | 0.0 |
| Medulloblastom/Cerebellum | | | carcinoma | | |
| 94907 D283 Med | 0.0 | 0.0 | 94957 Ramos Stimulated with | 0.0 | 0.0 |
| Medulloblastoma/Cerebellum | | | PMA/ionomycin 6 h | | |
| 94908 PFSK-1 Primitive | 0.0 | 1.4 | 94958 Ramos Stimulated with | 0.0 | 0.0 |
| Neuroectodermal/Cerebellum | | | PMA/ionomycin 14 h | | |
| 94909 XF-498 CNS | 0.0 | 0.0 | 94962 MEG-01 Chronic | 0.0 | 0.0 |
| | | | myelogenous leukemia | | |
| | | | (megokaryoblast) | | |
| 94910 SNB-78 CNS/glioma | 0.0 | 0.0 | 94963 Raji Burkitt's | 0.0 | 0.0 |
| | | | lymphoma | | |
| 94911 SF-268 CNS/glioblastoma | 0.0 | 0.0 | 94964 Daudi Burkitt's | 0.0 | 0.0 |
| | | | lymphoma | | |
| 94912 T98G Glioblastoma | 0.0 | 0.0 | 94965 U266 B-cell | 14.7 | 17.6 |
| | | | plasmacytoma/myeloma | | |
| 96776 SK-N-SH Neuroblastoma | 0.0 | 0.0 | 94968 CA46 Burkitt's | 0.0 | 0.0 |
| (metastasis) | | | lymphoma | | |
| 94913 SF-295 CNS/glioblastoma | 0.0 | 0.0 | 94970 RL non-Hodgkin's B- | 0.0 | 0.0 |
| | | | cell lymphoma | | |
| 94914 Cerebellum | 0.0 | 0.0 | 94972 JM1 pre-B-cell | 0.0 | 0.0 |
| | | | lymphoma/leukemia | | |
| 96777 Cerebellum | 1.2 | 0.0 | 94973 Jurkat T cell leukemia | 0.0 | 0.0 |
| 94916 NCI-H292 | 0.0 | 0.0 | 94974 TF-1 Erythroleukemia | 0.0 | 0.0 |
| Mucoepidermoid lung carcinoma | | | | | |
| 94917 DMS-114 Small cell lung | 0.0 | 0.0 | 94975 HUT 78 T-cell | 0.0 | 0.0 |
| cancer | | | lymphoma | | |
| 94918 DMS-79 Small cell lung | 100.0 | 100.0 | 94977 U937 Histiocytic | 0.0 | 0.0 |
| cancer/neuroendocrine | | | lymphoma | | |
| 94919 NCI-H146 Small cell lung | 0.0 | 0.0 | 94980 KU-812 Myelogenous | 0.0 | 0.0 |
| cancer/neuroendocrine | | | leukemia | | |
| 94920 NCI-H526 Small cell lung | 0.0 | 0.0 | 769-P- Clear cell renal | 0.0 | 0.0 |
| cancer/neuroendocrine | | | carcinoma | | |
| 94921 NCI-N417 Small cell lung | 0.0 | 0.0 | 94983 Caki-2 Clear cell renal | 0.8 | 1.9 |
| cancer/neuroendocrine | | | carcinoma | | |
| 94923 NCI-H82 Small cell lung | 0.0 | 0.0 | 94984 SW 839 Clear cell renal | 0.0 | 0.0 |
| cancer/neuroendocrine | | | carcinoma | | |
| 94924 NCI-H157 Squamous cell | 0.0 | 0.0 | 94986 G401 Wilms' tumor | 0.0 | 0.0 |
| lung cancer (metastasis) | | | | | |
| 94925 NCI-H1155 Large cell | 0.0 | 0.0 | 94987 Hs766T Pancreatic | 1.2 | 5.5 |
| lung cancer/neuroendocrine | | | carcinoma (LN metastasis) | | |
| 94926 NCI-H1299 Large cell | 0.0 | 0.0 | 94988 CAPAN-1 Pancreatic | 0.0 | 0.0 |
| lung cancer/neuroendocrine | | | adenocarcinoma (liver | | |
| | | | metastasis) | | |
| 94927 NCI-H727 Lung carcinoid | 0.0 | 0.0 | 94989 SU86.86 Pancreatic | 0.0 | 0.0 |
| | | | carcinoma (liver metastasis) | | |
| 94928 NCI-UMC-11 Lung | 0.0 | 4.9 | 94990 BxPC-3 Pancreatic | 0.0 | 2.9 |
| carcinoid | | | adenocarcinoma | | |
| 94929 LX-1 Small cell lung | 3.2 | 3.7 | 94991 HPAC Pancreatic | 1.2 | 5.1 |
| cancer | | | adenocarcinoma | | |
| 94930 Colo-205 Colon cancer | 7.5 | 29.3 | 94992 MIA PaCa-2 Pancreatic | 0.0 | 0.0 |
| | | | carcinoma | | |
| 94931 KM12 Colon cancer | 0.2 | 1.6 | 94993 CFPAC-1 Pancreatic | 29.1 | 47.6 |
| | | | ductal adenocarcinoma | | |
| 94932 KM20L2 Colon cancer | 1.2 | 0.0 | 94994 PANC-1 Pancreatic | 0.0 | 0.0 |
| | | | epithelioid ductal carcinoma | | |
| 94933 NCI-H716 Colon cancer | 0.0 | 0.0 | 94996 T24 Bladder carcinma | 0.0 | 0.0 |
| | | | (transitional cell | | |
| 94935 SW-48 Colon | 0.0 | 0.0 | 5637- Bladder carcinoma | 0.0 | 0.0 |
| adenocarcinoma | | | | | |
| 94936 SW1116 Colon | 0.0 | 0.0 | 94998 HT-1197 Bladder | 5.1 | 7.6 |
| adenocarcinoma | | | carcinoma | | |
| 94937 LS 174T Colon | 1.1 | 0.0 | 94999 UM-UC-3 Bladder | 0.0 | 0.0 |
| adenocarcinoma | | | carcinma (transitional cell) | | |
| 94938 SW-948 Colon | 0.0 | 0.0 | 95000 A204 | 0.0 | 0.0 |
| adenocarcinoma | | | Rhabdomyosarcoma | | |
| 94939 SW-480 Colon | 0.0 | 0.0 | 95001 HT-1080 Fibrosarcoma | 0.0 | 0.0 |
| adenocarcinoma | | | | | |
| 94940 NCI-SNU-5 Gastric | 0.0 | 1.1 | 95002 MG-63 Osteosarcoma | 0.0 | 0.0 |
| carcinoma | | | (bone) | | |
| KATO III- Gastric carcinoma | 0.0 | 1.9 | 95003 SK-LMS-1 | 0.0 | 0.0 |
| | | | Leiomyosarcoma (vulva) | | |
| 94943 NCI-SNU-16 Gastric | 2.2 | 0.0 | 95004 SJRH30 | 0.0 | 0.0 |
| carcinoma | | | Rhabdomyosarcoma (met to | | |
| | | | bone marrow) | | |
| 94944 NCI-SNU-1 Gastric | 0.0 | 0.0 | 95005 A431 Epidermoid | 1.8 | 1.6 |
| carcinoma | | | carcinoma | | |
| 94946 RF-1 Gastric | 0.0 | 0.0 | 95007 WM266-4 Melanoma | 0.0 | 0.0 |
| adenocarcinoma | | | | | |
| 94947 RF-48 Gastric | 0.0 | 0.0 | DU 145- Prostate carcinoma | 0.0 | 0.0 |
| adenocarcinoma | | | (brain metastasis) | | |
| 96778 MKN-45 Gastric | 0.0 | 0.0 | 95012 MDA-MB-468 Breast | 1.1 | 0.0 |
| carcinoma | | | adenocarcinoma | | |
| 94949 NCI-N87 Gastric | 0.0 | 3.8 | SCC-4- Squamous cell | 0.0 | 0.0 |
| carcinoma | | | carcinoma of tongue | | |
| 94951 OVCAR-5 Ovarian | 29.7 | 62.0 | SCC-9- Squamous cell | 0.0 | 0.0 |
| carcinoma | | | carcinoma of tongue | | |
| 94952 RL95-2 Uterine carcinoma | 19.3 | 21.8 | SCC-15- Squamous cell | 0.0 | 0.0 |
| | | | carcinoma of tongue | | |
| 94953 HelaS3 Cervical | 0.0 | 0.0 | 95017 CAL 27 Squamous cell | 0.0 | 0.0 |
| adenocarcinoma | | | carcinoma of tongue | | |

TABLE BLL
Panel 4D
Column A - Rel. Exp. (%) Ag1590, Run 152061102
Column B - Rel. Exp. (%) Ag1590, Run 155330411
Column C - Rel. Exp. (%) Ag1918, Run 147288180
Column D - Rel. Exp. (%) Ag2899, Run 159633215

| Tissue Name | A | B | C | D |
|---|---|---|---|---|
| Secondary Th1 act | 0.0 | 0.0 | 0.0 | 0.0 |
| Secondary Th2 act | 0.0 | 0.0 | 0.0 | 4.3 |
| Secondary Tr1 act | 0.0 | 0.0 | 0.0 | 6.6 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primaiy Th2 act | 0.0 | 0.0 | 0.0 | 0.0 |
| Primary Tr1 act | 0.0 | 0.0 | 0.0 | 0.0 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 lymphocyte act | 0.0 | 0.0 | 0.0 | 0.0 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 0.0 | 0.0 | 0.0 |
| Secondary CD8 lymphocyte act | 0.0 | 0.0 | 0.0 | 0.0 |
| CD4 lymphocyte none | 0.0 | 0.0 | 0.0 | 0.0 |
| 2ry Th1/Th2/Tr1 anti-CD95 CH11 | 0.0 | 0.0 | 0.0 | 0.0 |
| LAK cells rest | 0.0 | 0.0 | 0.0 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.0 |
| NK cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 6.3 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 0.0 | 0.0 | 0.0 | 0.0 |
| B lymphocytes CD40L and IL-4 | 0.0 | 0.0 | 0.0 | 0.0 |
| EOL-1 dbcAMP | 0.0 | 0.0 | 0.0 | 0.0 |
| EOL-1 dbcAMP PMA/ionomycin | 0.0 | 0.0 | 0.0 | 0.0 |
| Dendritic cells none | 0.0 | 0.0 | 0.0 | 0.0 |
| Dendritic cells LPS | 0.0 | 0.0 | 0.0 | 0.0 |
| Dendnitic cells anti-CD40 | 0.0 | 0.0 | 0.0 | 0.0 |
| Monocytes rest | 0.0 | 0.0 | 0.0 | 12.9 |
| Monocytes LPS | 0.0 | 0.0 | 0.0 | 0.0 |
| Macrophages rest | 0.0 | 0.0 | 0.0 | 0.0 |
| Macrophages LPS | 0.0 | 0.0 | 0.0 | 0.0 |
| HUVEC none | 0.0 | 0.0 | 0.0 | 0.0 |
| HUVEC starved | 0.0 | 0.0 | 0.0 | 0.0 |
| HUVEC IL-1beta | 0.0 | 0.0 | 0.0 | 0.0 |
| HUVEC IFN gamma | 0.0 | 0.0 | 0.0 | 0.0 |
| HUVEC TNF alpha + IFN gamma | 0.0 | 0.0 | 0.0 | 0.0 |
| HUVEC TNF alpha + IL4 | 0.0 | 0.0 | 0.0 | 0.0 |
| HUVEC IL-11 | 0.0 | 0.0 | 0.0 | 0.0 |
| Lung Microvascular EC none | 0.0 | 0.0 | 0.0 | 0.0 |
| Lung Microvascular EC TNFalpha + | 0.0 | 0.0 | 0.0 | 0.0 |
| IL-1beta | | | | |
| Microvascular Dermal EC none | 0.0 | 0.0 | 0.0 | 0.0 |
| Microsvasular Dermal EC TNFalpha + | 0.0 | 0.0 | 0.0 | 0.0 |
| IL-1beta | | | | |
| Bronchial epithelium TNFalpha + IL-1beta | 13.9 | 0.0 | 30.1 | 3.4 |
| Small airway epithelium none | 0.0 | 0.0 | 0.0 | 0.0 |
| Small airway epithelium TNFalpha + | 0.0 | 0.0 | 0.0 | 0.0 |
| IL-1beta | | | | |
| Coronery artery SMC rest | 0.0 | 0.0 | 0.0 | 0.0 |
| Coronety artery SMG TNFalpha + IL-1beta | 0.0 | 0.0 | 0.0 | 0.0 |
| Astrocytes rest | 0.0 | 0.0 | 0.0 | 0.0 |
| Astrocytes TNFalpha + IL-1beta | 0.0 | 0.0 | 0.0 | 0.0 |
| KU-812 (Basophil) rest | 0.0 | 0.0 | 0.0 | 0.0 |
| KU-812 (Basophil) PMA/ionomycin | 10.8 | 0.0 | 0.0 | 5.4 |
| CCD1106 (Keratinocytes) none | 0.0 | 0.0 | 0.0 | 0.0 |
| CCD1106 (Keratinocytes) TNFalpha + | 0.0 | 0.0 | 0.0 | 0.0 |
| IL-1beta | | | | |
| Liver cirrhosis | 22.7 | 80.1 | 63.7 | 100.0 |
| Lupus kidney | 0.0 | 7.4 | 0.0 | 0.0 |
| NCI-H292 none | 60.3 | 92.7 | 36.3 | 28.9 |
| NCI-H292 IL-4 | 55.9 | 6.5 | 28.7 | 14.3 |
| NCI-H292 IL-9 | 100.0 | 41.8 | 40.1 | 29.7 |
| NCI-H292 IL-13 | 35.8 | 6.7 | 9.4 | 23.7 |
| NCI-H292 IFN gamma | 0.0 | 23.8 | 1.2 | 0.0 |
| HPAEC none | 0.0 | 0.0 | 0.0 | 0.0 |
| HPAEC TNF alpha + IL-1 beta | 0.0 | 0.0 | 0.0 | 0.0 |
| Lung fibroblast none | 0.0 | 0.0 | 0.0 | 0.0 |
| Lung fibroblast TNF alpha + IL-1 beta | 0.0 | 0.0 | 0.0 | 0.0 |
| Lung fibroblast IL-4 | 0.0 | 0.0 | 0.0 | 0.0 |
| Lung fibroblast IL-9 | 0.0 | 0.0 | 0.0 | 0.0 |
| Lung fibroblast IL-13 | 0.0 | 0.0 | 0.0 | 0.0 |
| Lung fibroblast IFN gamma | 0.0 | 0.0 | 0.0 | 0.0 |
| Dermal fibroblast CCD1070 rest | 0.0 | 0.0 | 0.0 | 0.0 |
| Dermal fibroblast CCD1070 TNF alpha | 0.0 | 0.0 | 0.0 | 0.0 |
| Dermal fibroblast CCD1070 IL-1 beta | 0.0 | 0.0 | 0.0 | 0.0 |
| Dermal fibroblast IFN gamma | 0.0 | 0.0 | 0.0 | 0.0 |
| Dermal fibroblast IL-4 | 0.0 | 0.0 | 0.0 | 0.0 |
| IBD Colitis 2 | 0.0 | 0.0 | 0.0 | 0.0 |
| IBD Crohn's | 13.8 | 0.0 | 0.0 | 0.0 |
| Colon | 79.6 | 100.0 | 100.0 | 74.7 |
| Lung | 77.9 | 66.0 | 53.6 | 85.3 |
| Thymus | 62.0 | 21.6 | 0.7 | 23.0 |
| Kidney | 0.0 | 0.0 | 0.0 | 0.0 |

Ardais Breast1.0 Summary: Ag720 Expression of this gene was highest in a breast cancer sample (CT=27.1). Significant expression of this gene was detected in 45/64 breast cancer samples but only 1/7 normal breast samples. Gene or protein expression levels are useful for the detection of breast cancer. Therapeutic modulation of the activity of this gene or its protein product using nucleic acid, protein, antibody or small molecule drugs are useful in the treatment of breast cancer.

This gene encodes a protein with homology to mastocytoma protease precursor. Mast cell tryptase is a secretory granule associated serine protease with trypsin-like specificity. It is released extracellularly during mast cell degranulation. Mast cells (MC) have been associated with diverse human cancers. The primary function of these cells is to store and release a number of biologically active mediators, including the serine proteases tryptase and chymase. These proteases have been closely related with angiogenesis and tumor invasion, two critical steps during tumor progression. Malignant breast tumors have two to three times more tryptase-containing than chymase-containing mast cells, with the number of mast cells with trptase activity being significantly higher (p<0.02) than in benign lesions. In malignant lesions, tryptase-containing mast cells were concentrated at the tumor edge, i.e. the invasion zone (Kankkunen J P, Harvima I T, Naukkarinen A. Quantitative analysis of tryptase and chymase containing mast cells in benign and malignant breast lesions. Int J Cancer. 1997 Jul. 29; 72(3): 385-8). It is therefore likely that this protein has a role in tumor invasion and metastasis.

Panel 1.3D Summary: Ag1590/2899 The expression of this gene was assessed in four independent runs using two different probe/primer sets. All of the runs show excellent concordance. The expression of this gene appears to be highest in a sample derived from an ovarian cancer cell line (OVCAR-5) (CTs=31-32). In addition, there appears to be substantial expression associated with a colon cancer cell line, a gastric cancer cell line and pituitary tissue. Thus, the expression of this gene could be used to distinguish OVCAR-5 cells from the other samples in the panel. Moreover, therapeutic modulation of this gene, through the use of small molecule drugs, protein therapeutics or antibodies might be of benefit in the treatment of ovarian cancer, gastric cancer or colon cancer

Panel 2D Summary: Ag720/1590/2899 The expression of this gene was assessed in five independent runs in panel 2D using three different primer/probe paris. There is excellent concordance of between these runs. The expression of this gene was highest and exclusive to breast cancer samples (CTs=26-28). Thus, the expression of this gene could be used to distinguish breast cancer samples from other samples in the panel. Moreover, therapeutic modulation of this gene, through the use of small molecule drugs, protein therapeutics or antibodies might be of benefit in the treatment of breast cancer.

Panel 3D Summary: Ag720/2899 The expression of this gene was highest and almost exclusive to a sample derived from a lung cancer cell line (DMS-79)(CTs=29-31). In addition, there was low but substantial expression associated with samples derived from an ovarian cancer cell line, a uterine cancer cell line and a pancreatic cancer cell line. The expression of this gene or expressed protein is useful in the detection of lung cancer. Therapeutic modulation of this gene, expressed protein and/or use of antibodies or small molecule drugs targeting the gene or gene product are useful in the treatment of lung cancer, ovarian cancer, pancreatic cancer or uterine cancer.

Panel 4D Summary: Ag1590/Ag1918/Ag2899 This gene, a tryptase homolog, was expressed at significant levels in IL-9-activated NCI-H292 cells, pulmonary mucoepidermoid cells. Colon, lung, and thymus tissues also showed low levels of expression of this gene. The expression in lung and in the activated NCI-H292 cell line—often used as a model for airway epithelium—is consistent with published reports of tryptase in the lung (Walls A F, Bennett A R, Godfrey R C, Holgate S T, Church M K. Mast cell tryptase and histamine concentrations in bronchoalveolar lavage fluid from patients with interstitial lung disease. Clin Sci (Lond) 1991 August; 81(2):183-8). In addition, tryptase has been shown to be up-regulated in lungs affected by disease and specifically in COPD (Grashoff W F, Sont J K, Sterk P J, Hiemstra P S, de Boer W I, Stolk J, Han J, van Krieken J M. Chronic obstructive pulmonary disease: role of bronchiolar mast cells and macrophages. Am J Pathol 1997 December; 151(6):1785-90). Tryptase has also been implicated in the recruitment of granulocytes and epithelial repair (Cairns J A, Walls A F. Mast cell tryptase is a mitogen for epithelial cells. Stimulation of IL-8 production and intercellular adhesion molecule-1 expression. J Immunol 1996 Jan. 1; 156(1):275-83). Based On these observations, small molecule antagonists or antagonist antibodies are useful in the reduction or elimination of symptoms in patients with lung diseases including asthma, allergy, or chronic obstructive pulmonary disease.

BM. CG59693-01 and CG59693-03: 20 Alpha-Hydroxysteroid Dehydrogenase.

Expression of genes CG59693-01 and CG59693-03 was assessed using the primer-probe set Ag3562, described in Table BMA. Results of the RTQ-PCR runs are shown in Tables BMB, BMC, BMD, BME, BMF, BMG, BMH and BMI. CG59693-03 represents a full-length physical clone of the CG59693-01 gene.

TABLE BMA
Probe Name Ag3562

| | | | | SEQ |
|---|---|---|---|---|
| | | | Start | ID |
| Primers | Sequences | Length | Position | No |
| Forward | 5′-ctggccaagagctacaatga-3′ | 20 | 802 | 1437 |
| Probe | TET-5′-catcagacagaacgtgcaggtgtttg-3′- | 26 | 828 | 1438 |
| | TAMRA | | | |
| Reverse | 5′-aggccatctatggctttcat-3′ | 20 | 877 | 1439 |

TABLE BMB
Ardais Panel v.1.0
Column A - Rel. Exp. (%) Ag3562, Run 263525399

| Tissue Name | A |
|---|---|
| 136799 Lung cancer(362) | 52.9 |
| 136800 Lung NAT(363) | 0.8 |
| 136813 Lung cancer(372) | 100.0 |
| 136814 Lung NAT(373) | 0.4 |
| 136815 Lung cancer(374) | 1.1 |
| 136816 Lung NAT(375) | 1.6 |
| 136791 Lung cancer(35A) | 0.4 |
| 136795 Lung cancer(35E) | 1.6 |
| 136797 Lung cancer(360) | 0.4 |
| 136794 lung NAT(35D) | 1.2 |
| 136818 Lung NAT(377) | 0.5 |
| 136787 lung cancer(356) | 0.1 |
| 136788 lung NAT(357) | 1.1 |
| 136806 Lung cancer(36B) | 0.1 |
| 136807 Lung NAT(36C) | 0.4 |
| 136789 lung cancer(358) | 0.4 |
| 136802 Lung cancer(365) | 1.6 |
| 136803 Lung cancer(368) | 0.5 |
| 136804 Lung cancer(369) | 1.4 |
| 136811 Lung cancer(370) | 64.2 |
| 136810 Lung NAT(36F) | 3.9 |

TABLE BMC
CNS_neurodegeneration_v1.0
Column A - Rel. Exp. (%) Ag3562, Run 210629741
Column B - Rel. Exp. (%) Ag3562, Run 224078542

| Tissue Name | A | B | Tissue Name | A | B |
|---|---|---|---|---|---|
| AD 1 Hippo | 100.0 | 100.0 | AH3 4624 | 16.5 | 24.3 |
| AD 2 Hippo | 37.9 | 33.9 | AH3 4640 | 22.7 | 21.3 |
| AD 3 Hippo | 28.1 | 35.6 | AD 1 Occipital Ctx | 42.0 | 65.5 |
| AD 4 Hippo | 26.8 | 25.7 | AD 2 Occipital Ctx (Missing) | 0.7 | 1.2 |
| AD 5 Hippo | 37.1 | 41.5 | AD 3 Occipital Ctx | 32.8 | 27.7 |
| AD 6 Hippo | 76.8 | 66.4 | AD 4 Occipital Ctx | 20.6 | 11.2 |
| Control 2 Hippo | 30.6 | 32.3 | AD 5 Occipital Ctx | 26.1 | 27.0 |
| Control 4 Hippo | 30.8 | 34.9 | AD 5 Occipital Ctx | 39.0 | 48.0 |
| Control (Path) 3 Hippo | 24.1 | 21.0 | Control 1 Occipital Ctx | 18.2 | 16.6 |
| AD 1 Temporal Ctx | 57.8 | 64.2 | Control 2 Occipital Ctx | 16.7 | 24.0 |
| AD 2 Temporal Ctx | 34.4 | 29.9 | Control 3 Occipital Ctx | 18.4 | 19.8 |
| AD 3 Temporal Ctx | 25.7 | 27.7 | Control 4 Occipital Ctx | 20.4 | 22.1 |
| AD 4 Temporal Ctx | 23.0 | 24.1 | Control (Path) 1 Occipital Ctx | 21.8 | 18.0 |
| AD 5 Inf Temporal Ctx | 33.7 | 44.1 | Control (Path) 2 Occipital Ctx | 11.9 | 15.7 |
| AD 5 Sup Temporal Ctx | 54.7 | 54.3 | Control (Path) 3 Occipital Ctx | 34.4 | 22.1 |
| AD 6 Inf Temporal Ctx | 47.3 | 48.0 | Control (Path) 4 Occipital Ctx | 18.0 | 18.6 |
| AD 6 Sup Temporal Ctx | 45.4 | 46.7 | Control 1 Parietal Ctx | 16.5 | 19.1 |
| Control 1 Temporal Ctx | 22.8 | 23.8 | Control 2 Parietal Ctx | 62.9 | 55.9 |
| Control 2 Temporal Ctx | 18.4 | 29.1 | Control 3 Parietal Ctx | 35.8 | 29.7 |
| Control 3 Temporal Ctx | 25.5 | 19.3 | Control (Path) 1 Parietal Ctx | 24.0 | 24.7 |
| Control 3 Temporal Ctx | 18.2 | 16.8 | Control (Path) 2 Parietal Ctx | 15.9 | 15.6 |
| AH3 3975 | 21.5 | 22.1 | Control (Path) 3 Parietal Ctx | 22.1 | 26.1 |
| AH3 3954 | 17.4 | 17.3 | Control (Path) 4 Parietal Ctx | 23.0 | 24.1 |

TABLE BMD
General_screening_panel_v1.4
Column A - Rel. Exp. (%) Ag3562, Run 217240778

| Tissue Name | A |
|---|---|
| Adipose | 2.1 |
| Melanoma* Hs688(A).T | 0.2 |
| Melanoma* Hs688(B).T | 0.3 |
| Melanoma* M14 | 0.0 |
| Melanoma* LOXIMVI | 0.0 |
| Melanoma* SK-MEL-5 | 0.0 |
| Squamous cell carcinoma SCC-4 | 0.5 |
| Testis Pool | 0.2 |
| Prostate ca.* (bone met) PC-3 | 0.0 |
| Prostate Pool | 0.1 |
| Placenta | 0.0 |
| Uterus Pool | 0.1 |
| Ovarian ca. OVCAR-3 | 0.0 |
| Ovarian ca. SK-OV-3 | 11.8 |
| Ovarian ca. OVCAR-4 | 0.0 |
| Ovarian ca. OVCAR-5 | 0.3 |
| Ovarian ca. IGROV-1 | 0.5 |
| Ovarian ca. OVCAR-8 | 0.2 |
| Ovary | 0.2 |
| Breast ca. MCF-7 | 0.5 |
| Breast ca. MDA-MB-231 | 1.1 |
| Breast ca. BT 549 | 1.2 |
| Breast ca. T47D | 0.6 |
| Breast ca. MDA-N | 0.0 |
| Breast Pool | 0.1 |
| Trachea | 1.3 |
| Lung | 0.2 |
| Fetal Lung | 0.3 |
| Lung ca. NCI-N417 | 0.0 |
| Lung ca. LX-1 | 1.6 |
| Lung ca. NCI-H146 | 1.0 |
| Lung ca. SHP-77 | 14.7 |
| Lung ca. A549 | 100.0 |
| Lung ca. NCI-H526 | 0.0 |
| Lung ca. NCI-H23 | 0.2 |
| Lung ca. NCI-H460 | 11.1 |
| Lung ca. HOP-62 | 0.1 |
| Lung ca. NCI-H522 | 0.6 |
| Liver | 0.7 |
| Fetal Liver | 4.1 |
| Liver ca. HepG2 | 2.4 |
| Kidney Pool | 0.2 |
| Fetal Kidney | 0.1 |
| Renal ca. 786-0 | 0.3 |
| Renal ca. A498 | 11.2 |
| Renal ca. ACHN | 0.2 |
| Renal ca. UO-31 | 0.1 |
| Renal ca. TK-10 | 1.5 |
| Bladder | 0.8 |
| Gastric ca. (liver met.) NCI-N87 | 0.1 |
| Gastric ca. KATO III | 0.4 |
| Colon ca. SW-948 | 0.6 |
| Colon ca. SW480 | 0.1 |
| Colon ca.* (SW480 met) SW620 | 0.5 |
| Colon ca. HT29 | 0.5 |
| Colon ca. HCT-116 | 0.0 |
| Colon ca. CaCo-2 | 2.6 |
| Colon cancer tissue | 0.4 |
| Colon ca. SW1116 | 0.0 |
| Colon ca. Colo-205 | 3.0 |
| Colon ca. SW-48 | 0.9 |
| Colon Pool | 0.1 |
| Small Intestine Pool | 0.1 |
| Stomach Pool | 0.1 |
| Bone Marrow Pool | 0.1 |
| Fetal Heart | 0.0 |
| Heart Pool | 0.1 |
| Lymph Node Pool | 0.2 |
| Fetal Skeletal Muscle | 0.1 |
| Skeletal Muscle Pool | 0.7 |
| Spleen Pool | 0.0 |
| Thymus Pool | 0.2 |
| CNS cancer (glio/astro) | 0.9 |
| U87-MG | |
| CNS cancer (glio/astro) | 1.0 |
| U-118-MG | |
| CNS cancer (neuro; met) | 0.3 |
| SK-N-AS | |
| CNS cancer (astro) SF-539 | 0.1 |
| CNS cancer (astro) SNB-75 | 10.2 |
| CNS cancer (glio) SNB-19 | 0.4 |
| CNS cancer (glio) SF-295 | 4.0 |
| Brain (Amygdala) Pool | 0.2 |
| Brain (cerebellum) | 0.1 |
| Brain (fetal) | 0.5 |
| Brain (Hippocampus) Pool | 0.1 |
| Cerebral Cortex Pool | 0.1 |
| Brain (Substantia nigra) Pool | 0.2 |
| Brain (Thalamus) Pool | 0.2 |
| Brain (whole) | 0.4 |
| Spinal Cord Pool | 0.3 |
| Adrenal Gland | 0.2 |
| Pituitary gland Pool | 0.0 |
| Salivary Gland | 0.1 |
| Thyroid (female) | 0.1 |
| Pancreatic ca. CAPAN2 | 0.2 |
| Pancreas Pool | 0.2 |

TABLE BME
HASS Panel v1.0
Column A - Rel. Exp. (%) Ag3562, Run 276044499

| Tissue Name | A |
|---|---|
| MCF-7 C1 | 8.5 |
| MCF-7 C2 | 12.0 |
| MCF-7 C3 | 15.7 |
| MGF-7 C4 | 13.2 |
| MGF-7 C5 | 20.2 |
| MCF-7 C6 | 10.4 |
| MCF-7 C7 | 8.8 |
| MGF-7 C9 | 6.9 |
| MCF-7 C10 | 10.2 |
| MCF-7 C11 | 5.8 |
| MCF-7 C12 | 8.7 |
| MGF-7 C13 | 7.6 |
| MCF-7 C15 | 3.6 |
| MGF-7 C16 | 13.2 |
| MCF-7 C17 | 9.8 |
| T24 D1 | 32.8 |
| T24 D2 | 12.2 |
| T24 D3 | 26.4 |
| T24 D4 | 48.3 |
| T24 D5 | 18.2 |
| T24 D6 | 0.2 |
| T24 D7 | 1.0 |
| T24 D9 | 0.0 |
| T24 D10 | 23.7 |
| T24 D11 | 2.7 |
| T24 D12 | 0.1 |
| T24 D13 | 0.3 |
| T24 D15 | 0.3 |
| T24 D16 | 0.2 |
| T24 D17 | 0.5 |
| CAPaN B1 | 4.4 |
| CAPaN B2 | 6.3 |
| CAPaN B3 | 4.3 |
| CAPaN B4 | 3.4 |
| CAPaN B5 | 14.9 |
| CAPaN B6 | 2.1 |
| CAPaN B7 | 1.8 |
| CAPaN B8 | 10.7 |
| CAPaN B9 | 2.6 |
| CAPaN B10 | 6.4 |
| CAPaN B11 | 16.3 |
| CAPaN B12 | 1.7 |
| CAPaN B13 | 1.3 |
| CAPaN B14 | 7.4 |
| CAPaN B15 | 11.2 |
| CAPaN B16 | 4.2 |
| CAPaN B17 | 14.2 |
| U87-MG F1 (B) | 19.6 |
| U87-MG F2 | 5.2 |
| U87-MG F3 | 15.8 |
| U87-MG F4 | 10.2 |
| U87-MG F5 | 40.3 |
| U87-MG F6 | 92.7 |
| U87-MG F7 | 5.9 |
| U87-MG F8 | 9.1 |
| U87-MG F9 | 1.9 |
| U87-MG F10 | 57.4 |
| U87-MG F11 | 100.0 |
| U87-MG F12 | 30.4 |
| U87-MG F13 | 10.2 |
| U87-MG F14 | 16.7 |
| U87-MG F15 | 9.5 |
| U87-MG F16 | 66.4 |
| U87-MG F17 | 67.4 |
| LnCAP A1 | 0.2 |
| LnCAP A2 | 0.2 |
| LnCAP A3 | 0.1 |
| LnCAP A4 | 0.1 |
| LnCAP A5 | 0.2 |
| LnCAP A6 | 0.2 |
| LnCAP A7 | 0.4 |
| LnCAP A8 | 0.6 |
| LnCAP A9 | 0.8 |
| LnCAP A10 | 0.2 |
| LnCAP A11 | 0.4 |
| LnCAP A12 | 0.1 |
| LnCAP A13 | 0.0 |
| LnCAP A14 | 0.2 |
| LnCAP A15 | 0.3 |
| LnCAP A16 | 0.2 |
| LnCAP A17 | 0.5 |
| Primary Astrocytes | 0.4 |
| Primary Renal Proximal Tubule Epithelial cell A2 | 25.0 |
| Primary melanocytes A5 | 1.7 |
| 126443 - 341 medullo | 0.1 |
| 126444 - 487 medullo | 0.0 |
| 126445 - 425 medullo | 0.0 |
| 126446 - 690 medullo | 0.2 |
| 126447 - 54 adult glioma | 3.8 |
| 126448 - 245 adult glioma | 0.0 |
| 126449 - 317 adult glioma | 0.0 |
| 126450 - 212 glioma | 0.1 |
| 126451 - 456 glioma | 0.0 |

TABLE BMF
Oncology cell_line_screening_panel_v3.1
Column A - Rel. Exp. (%) Ag3562, Run 222546381

| Tissue Name | A |
|---|---|
| 94905 Daoy Medulloblastoma/Cerebellum | 0.0 |
| 94906 TE671 Medulloblastom/Cerebellum | 0.2 |
| 94907 D283 Med Medulloblastoma/Cerebellum | 0.0 |
| 94908 PFSK-1 Primitive Neuroectodermal/Cerebellum | 0.1 |
| 94909 XF-498 CNS | 0.0 |
| 94910 SNB-78 CNS/glioma | 0.3 |
| 94911 SF-268 CNS/glioblastoma | 0.0 |
| 94912 T98G Glioblastoma | 33.2 |
| 96776 SK-N-SH Neuroblastoma (metastasis) | 0.0 |
| 94913 SF-295 CNS/glioblastoma | 2.3 |
| 94914 Cerebellum | 0.4 |
| 96777 Cerebellum | 0.1 |
| 94916 NCI-H292 Mucoepidermoid lung carcinoma | 0.3 |
| 94917 DMS-114 Small cell lung cancer | 0.0 |
| 94918 DMS-79 Small cell lung cancer/neuroendocrine | 0.0 |
| 94919 NCI-H146 Small cell lung cancer/neuroendocrine | 2.2 |
| 94920 NCI-H526 Small cell lung cancer/neuroendocrine | 0.0 |
| 94921 NCI-N417 Small cell lung cancer/neuroendocrine | 0.0 |
| 94923 NCI-H82 Small cell lung cancer/neuroendocrine carcinoma | 0.0 |
| 94924 NCI-H157 Squamous cell lung cancer (metastasis) | 0.0 |
| 94925 NCI-H1155 Large cell lung cancer/neuroendocrine | 0.0 |
| 94926 NCI-H1299 Large cell lung cancer/neuroendocrine | 0.0 |
| 94927 NCI-H727 Lung carcinoid | 1.3 |
| 94928 NCI-UMC-11 Lung carcinoid | 100.0 |
| 94929 LX-1 Small cell lung cancer | 1.6 |
| 94930 Colo-205 Colon cancer | 7.5 |
| 94931 KM12 Colon cancer | 0.1 |
| 94932 KM20L2 Colon cancer | 0.1 |
| 94933 NCI-H716 Colon cancer | 19.2 |
| 94935 SW-48 Colon adenocarcinoma | 2.3 |
| 94936 SW1116 Colon adenocarcinoma | 0.0 |
| 94937 LS 174T Colon adenocarcinoma | 0.9 |
| 94938 SW-948 Colon adenocarcinoma | 0.3 |
| 94939 SW-480 Colon adenocarcinoma | 0.5 |
| 94940 NCI-SNU-5 Gastric carcinoma | 0.0 |
| 112197 KATO III Stomach | 0.1 |
| 94943 NCI-SNU-16 Gastric carcinoma | 2.7 |
| 94944 NCI-SNU-1 Gastric carcinoma | 16.8 |
| 94946 RF-1 Gastric adenocarcinoma | 0.5 |
| 94947 RF-48 Gastric adenocarcinoma | 0.0 |
| 96778 MKN-45 Gastric carcinoma | 1.6 |
| 94949 NCI-N87 Gastric carcinoma | 0.8 |
| 94951 OVCAR-5 Ovarian carcinoma | 0.4 |
| 94952 RL95-2 Uterine carcinoma | 0.5 |
| 94953 HelaS3 Cervical adenocarcinoma | 0.9 |
| 94954 Ca Ski Cervical epidermoid carcinoma (metastasis | 0.3 |
| 94955 ES-2 Ovarian clear cell carcinoma | 0.0 |
| 94957 Ramos Stimulated with PMA/ionomycin 6h | 0.0 |
| 94958 Ramos Stimulated with PMA/ionomycin 14h | 0.0 |
| 94962 MEG-01 Chronic myelogenous leukemia (megokaryoblast) | 0.1 |
| 94963 Raji Burkitt's lymphoma | 0.0 |
| 94964 Daudi Burkitt's lymphoma | 0.0 |
| 94965 U266 B-cell plasmacytoma/myeloma | 0.0 |
| 94968 CA46 Burkitt's lymphoma | 0.0 |
| 94970 RL non-Hodgkin's B-cell lymphoma | 0.0 |
| 94972 JM1 pre-B-cell lymphoma/leukemia | 0.0 |
| 94973 Jurkat T cell leukemia | 0.0 |
| 94974 TF-1 Erythroleukemia | 1.6 |
| 94975 HUT 78 T-cell lymphoma | 0.0 |
| 94977 U937 Histiocytic lymphoma | 0.0 |
| 94980 KU-812 Myelogenous leukemia | 0.9 |
| 769-P- Clear cell renal carcinoma | 1.0 |
| 94983 Caki-2 Clear cell renal carcinoma | 63.7 |
| 94984 SW 839 Clear cell renal | 0.4 |
| 94986 G401 Wilms' tumor | 0.0 |
| 94987 Hs766T Pancreatic carcinoma (LN metastasis) | 0.2 |
| 94988 CAPAN-1 Pancreatic adenocarcinoma (liver metastasis) | 0.3 |
| 94989 SU86.86 Pancreatic carcinoma (liver metastasis) | 1.2 |
| 94990 BxPC-3 Pancreatic adenocarcinoma | 3.6 |
| 94991 HPAC Pancreatic adenocarcinoma | 3.1 |
| 94992 MIA PaCa-2 Pancreatic carcinoma | 0.0 |
| 94993 CFPAC-1 Pancreatic ductal adenocarcinoma | 3.9 |
| 94994 PANC-1 Pancreatic epitheliold ductal carcinoma | 0.0 |
| 94996 T24 Bladder carcinma (transitional cell | 2.0 |
| 5637- Bladder carcinoma | 0.1 |
| 94998 HT-1197 Bladder carcinoma | 0.0 |
| 94999 UM-UC-3 Bladder carcinma (transitional cell) | 0.1 |
| 95000 A204 Rhabdomyosarcoma | 0.8 |
| 95001 HT-1080 Fibrosarcoma | 0.0 |
| 95002 MG-63 Osteosarcoma (bone) | 0.0 |
| 95003 SK-LMS-1 Leiomyosarcoma (vulva) | 0.0 |
| 95004 SJRH30 Rhabdomyosarcoma (met to bone marrow) | 0.1 |
| 95005 A431 Epidermoid carcinoma | 3.8 |
| 95007 WM266-4 Melanoma | 0.1 |
| 112195 DU 145 Prostate | 0.1 |
| 95012 MDA-MB-468 Breast adenocarcinoma | 0.5 |
| 112196 SSC-4 Tongue | 0.4 |
| 112194 SSC-9 Tongue | 0.2 |
| 112191 SSC-15 Tongue | 0.3 |
| 95017 CAL 27 Squamous cell carcinoma of | 0.1 |
| tongue | |

TABLE BMG
Panel 2D
Column A - Rel. Exp. (%) Ag3562, Run 170858350

| Tissue Name | A |
|---|---|
| Normal Colon | 5.0 |
| CC Well to Mod Diff (OD03866) | 1.5 |
| CC Margin (OD03866) | 2.0 |
| CC Gr.2 rectosigmoid (OD03868) | 0.5 |
| CC Margin (OD03868) | 0.5 |
| CC Mod Duff (OD03920) | 0.5 |
| CC Margin (OD03920) | 1.2 |
| CC Gr.2 ascend colon (OD03921) | 2.1 |
| CC Margin (OD03921) | 1.4 |
| CC from Partial Hepatectomy (OD04309) Mets | 5.5 |
| Liver Margin (OD04309) | 27.0 |
| Colon mets to lung (OD04451-01) | 0.4 |
| Lung Margin (OD04451-02) | 1.8 |
| Normal Prostate 6546-1 | 1.3 |
| Prostate cancer (OD04410) | 0.4 |
| Prostate Margin (OD04410) | 0.5 |
| Prostate cancer (OD04720-01) | 0.6 |
| Prostate Margin (OD04720-02) | 1.6 |
| Normal Lung | 4.1 |
| Lung Met to Muscle (OD04286) | 41.8 |
| Muscle Margin (OD04286) | 1.7 |
| Lung Malignant cancer (OD03126) | 2.0 |
| Lung Margin (OD03126) | 2.1 |
| Lung cancer (OD04404) | 100.0 |
| Lung Margin (OD04404) | 1.7 |
| Lung cancer (OD04565) | 43.2 |
| Lung Margin (OD04565) | 0.7 |
| Lung cancer (OD04237-01) | 0.5 |
| Lung Margin (OD04237-02) | 2.2 |
| Ocular Mel Met to Liver (OD04310) | 0.1 |
| Liver Margin (OD04310) | 14.8 |
| Melanoma Metastasis | 0.1 |
| Lung Margin (OD04321) | 2.3 |
| Normal Kidney | 5.0 |
| Kidney Ca, Nuclear grade 2 (OD04338) | 29.1 |
| Kidney Margin (OD04338) | 3.1 |
| Kidney Ca Nuclear grade 1/2 (OD04339) | 3.7 |
| Kidney Margin (OD04339) | 5.1 |
| Kidney Ca, Clear cell type (OD04340) | 6.3 |
| Kidney Margin (OD04340) | 3.6 |
| Kidney Ca, Nuclear grade 3 (OD04348) | 0.1 |
| Kidney Margin (OD04348) | 1.8 |
| Kidney cancer (OD04622-01) | 1.9 |
| Kidney Margin (OD04622-03) | 1.5 |
| Kidney cancer (OD04450-01) | 12.0 |
| Kidney Margin (OD04450-03) | 4.5 |
| Kidney cancer 8120607 | 34.2 |
| Kidney Margin 8120608 | 2.3 |
| Kidney cancer 8120613 | 4.4 |
| Kidney Margin 8120614 | 4.2 |
| Kidney cancer 9010320 | 1.5 |
| Kidney Margin 9010321 | 2.6 |
| Normal Uterus | 0.4 |
| Uterine cancer 064011 | 0.5 |
| Normal Thyroid | 1.2 |
| Thyroid cancer | 0.1 |
| Thyroid cancer A302152 | 0.2 |
| Thyroid Margin A302153 | 1.6 |
| Normal Breast | 5.3 |
| Breast cancer | 0.2 |
| Breast cancer (OD04590-01) | 1.4 |
| Breast cancer Mets (OD04590-03) | 5.2 |
| Breast cancer Metastasis | 1.1 |
| Breast cancer | 0.3 |
| Breast cancer | 0.8 |
| Breast cancer 9100266 | 0.3 |
| Breast Margin 9100265 | 0.5 |
| Breast cancer A209073 | 0.6 |
| Breast Margin A209073 | 0.5 |
| Normal Liver | 11.9 |
| Liver cancer | 5.0 |
| Liver cancer 1025 | 14.7 |
| Liver cancer 1026 | 3.9 |
| Liver cancer 6004-T | 14.0 |
| Liver Tissue 6004-N | 20.9 |
| Liver cancer 6005-T | 3.7 |
| Liver Tissue 6005-N | 6.3 |
| Normal Bladder | 5.6 |
| Bladder cancer | 0.3 |
| Bladder cancer | 0.8 |
| Bladder cancer (OD04718-01) | 0.1 |
| Bladder Normal Adjacent (OD04718-03) | 0.9 |
| Normal Ovary | 0.4 |
| Ovarian cancer | 0.5 |
| Ovarian cancer (OD04768-07) | 0.6 |
| Ovary Margin (OD04768-08) | 0.3 |
| Normal Stomach | 5.5 |
| Gastric cancer 9060358 | 1.0 |
| Stomach Margin 9060359 | 11.7 |
| Gastric cancer 9060395 | 14.4 |
| Stomach Margin 9060394 | 15.3 |
| Gastric cancer 9060397 | 2.1 |
| Stomach Margin 9060396 | 6.7 |
| Gastric cancer 064005 | 7.6 |

TABLE BMH
Panel 4.1D
Column A - Rel. Exp. (%) Ag3562, Run 169990867

| Tissue Name | A | Tissue Name | A |
|---|---|---|---|
| Secondary Th1 act | 0.0 | HUVEC IL-1beta | 0.7 |
| Secondary Th2 act | 0.1 | HUVEC IFN gamma | 2.1 |
| Secondary Tr1 act | 0.2 | HUVEC TNF alpha + IFN gamma | 0.7 |
| Secondary Th1 rest | 0.2 | HUVEC TNF alpha + IL4 | 0.5 |
| Secondary Th2 rest | 0.1 | HUVEC IL-11 | 2.4 |
| Secondary Tr1 rest | 0.1 | Lung Microvascular EC none | 13.1 |
| Primary Th1 act | 0.0 | Lung Microvascular EC TNF alpha + IL-1 | 10.1 |
| | | beta | |
| Primary Th2 act | 0.1 | Microvascular Dermal EC none | 11.8 |
| Primary Tr1 act | 0.0 | Microsvasular Dermal EC TNF alpha + IL-1 | 12.8 |
| | | beta | |
| Primary Th1 rest | 0.4 | Bronchial epithelium TNF alpha + IL1beta | 92.7 |
| Primary Th2 rest | 0.2 | Small airway epithelium none | 29.9 |
| Primary Tr1 rest | 0.1 | Small airway epithelium TNF alpha + IL-1 | 50.3 |
| | | beta | |
| CD45RA CD4 lymphocyte act | 6.7 | Coronery artery SMC rest | 5.4 |
| CD45RO CD4 lymphocyte act | 0.4 | Coronery artery SMC TNF alpha + IL-1 beta | 7.0 |
| CD8 lymphocyte act | 0.3 | Astrocytes rest | 0.6 |
| Secondary CD8 lymphocyte rest | 0.0 | Astrocytes TNF alpha + IL-1 beta | 0.7 |
| Secondary CD8 lymphocyte act | 0.0 | KU-812 (Basophil) rest | 16.4 |
| CD4 lymphocyte none | 0.6 | KU-812 (Basophil) PMA/ionomycin | 33.2 |
| 2ry Th1/Th2/Tr1 anti-CD95 | 0.2 | CCD1106 (Keratinocytes) none | 2.9 |
| CH11 | | | |
| LAK cells rest | 0.5 | CCD1106 (Keratinocytes) TNF alpha + IL-1 | 2.3 |
| | | beta | |
| LAK cells IL-2 | 1.3 | Liver cirrhosis | 38.2 |
| LAK cells IL-2 + IL-12 | 0.5 | NCI-H292 none | 11.8 |
| LAK cells IL-2 + IFN gamma | 1.1 | NCI-H292 IL-4 | 7.1 |
| LAK cells IL-2 + IL-18 | 0.7 | NCI-H292 IL-9 | 22.5 |
| LAK cells PMA/ionomycin | 1.2 | NCI-H292 IL-13 | 5.2 |
| NK Cells IL-2 rest | 3.5 | NCI-H292 IFN gamma | 4.7 |
| Two Way MLR 3 day | 1.4 | HPAEC none | 5.1 |
| Two Way MLR 5 day | 0.6 | HPAEC TNF alpha + IL-1 beta | 7.6 |
| Two Way MLR 7 day | 0.3 | Lung fibroblast none | 6.8 |
| PBMC rest | 0.8 | Lung fibroblast TNF alpha + IL-1 beta | 48.6 |
| PBMC PWM | 15.0 | Lung fibroblast IL-4 | 7.5 |
| PBMC PHA-L | 0.3 | Lung fibroblast IL-9 | 7.2 |
| Ramos (B cell) none | 0.0 | Lung fibroblast IL-13 | 8.8 |
| Ramos (B cell) ionomycin | 0.0 | Lung fibroblast IFN gamma | 6.8 |
| B lymphocytes PWM | 0.2 | Dermal fibroblast CCD1070 rest | 13.9 |
| B lymphocytes CD40L and IL-4 | 0.3 | Dermal fibroblast CCD1070 TNF alpha | 19.9 |
| EOL-1 dbcAMP | 0.1 | Dermal fibroblast CCD1070 IL-1 beta | 24.7 |
| EOL-1 dbcAMP | 0.1 | Dermal fibroblast IFN gamma | 38.4 |
| PMA/ionomycin | | | |
| Dendritic cells none | 0.8 | Dermal fibroblast IL-4 | 100.0 |
| Dendritic cells LPS | 2.9 | Dermal Fibroblasts rest | 68.3 |
| Dendritic cells anti-CD40 | 0.6 | Neutrophilis TNFa + LPS | 0.2 |
| Monocytes rest | 0.1 | Neutrophilis rest | 0.1 |
| Monocytes LPS | 9.7 | Colon | 14.0 |
| Macrophages rest | 2.4 | Lung | 5.8 |
| Macrophages LPS | 2.9 | Thymus | 6.6 |
| HUVEC none | 1.0 | Kidney | 20.7 |
| HUVEC starved | 0.9 | | |

TABLE BMI
Panel 5 Islet
Column A - Rel. Exp. (%) Ag3562, Run 242386397

| Tissue Name | A | Tissue Name | A |
|---|---|---|---|
| 97457 Patient-02go adipose | 12.8 | 94709 Donor 2 AM - A adipose | 10.2 |
| 97476 Patient-07sk skeletal muscle | 12.4 | 94710 Donor 2 AM - B adipose | 7.1 |
| 97477 Patient-07ut uterus | 1.6 | 94711 Donor 2 AM - C adipose | 6.4 |
| 97478 Patient-07pl placenta | 0.3 | 94712 Donor 2 AD - A adipose | 17.9 |
| 99167 Bayer Patient 1 | 100.0 | 94713 Donor 2 AD - B adipose | 15.6 |
| 97482 Patient-08ut uterus | 0.7 | 94714 Donor 2 AD - C adipose | 19.1 |
| 97483 Patient-08pl placenta | 0.0 | 94742 Donor 3 U - A Mesenchymal | 2.0 |
| | | Stem Cells | |
| 97486 Patient-09sk skeletal muscle | 7.6 | 94743 Donor 3 U - B Mesenchymal | 3.4 |
| | | Stem Cells | |
| 97487 Patient-09ut uterus | 3.9 | 94730 Donor 3 AM - A adipose | 17.6 |
| 97488 Patient-09pl placenta | 0.4 | 94731 Donor 3 AM - B adipose | 7.6 |
| 97492 Patient-10ut uterus | 1.9 | 94732 Donor 3 AM - C adipose | 9.7 |
| 97493 Patient-10pl placenta | 0.5 | 94733 Donor 3 AD - A adipose | 32.5 |
| 97495 Patient-11go adipose | 7.4 | 94734 Donor 3 AD - B adipose | 6.7 |
| 97496 Patient-11sk skeletal muscle | 8.0 | 94735 Donor 3 AD - C adipose | 23.2 |
| 97497 Patient-11ut uterus | 2.8 | 77138 Liver HepG2untreated | 82.9 |
| 97498 Patient-11pl placenta | 0.1 | 73556 Heart Cardiac stromal cells | 1.9 |
| | | (primary) | |
| 97500 Patient-12go adipose | 11.3 | 81735 Small Intestine | 13.4 |
| 97501 Patient-12sk skeletal muscle | 33.2 | 72409 Kidney Proximal Convoluted | 3.6 |
| | | Tubule | |
| 97502 Patient-12ut uterus | 3.1 | 82685 Small intestine Duodenum | 2.5 |
| 97503 Patient-12p1 placenta | 0.4 | 90650 Adrenal Adrenocortical | 2.4 |
| | | adenoma | |
| 94721 Donor 2 U - A Mesenchymal | 3.8 | 72410 Kidney HRCE | 11.6 |
| Stem Cells | | | |
| 94722 Donor 2 U - B Mesenchymal | 4.3 | 72411 Kidney HRE | 1.3 |
| Stem Cells | | | |
| 94723 Donor 2 U - C Mesenchymal | 6.0 | 73139 Uterus Uterine smooth muscle | 0.4 |
| Stem Cells | | cells | |

Ardais Panel v.1.0 Summary: Ag3562 Highest expression of this gene was seen in lung cancer (CT=19.1). In addition, this gene was more highly expressed in three lung cancer samples than in the corresponding normal adjacent tissue. Thus, expression of this gene is useful as a marker of this cancer. Therapeutic modulation of this gene, expressed protein and/or use of antibodies or small molecule drugs targeting the gene or gene product are useful in the treatment of lung cancer.

CNS_neurodegeneration_v1.0 Summary: Ag3562 This panel confirms the expression of this gene at low levels in the brain in an independent group of individuals. This gene was found to be upregulated in the temporal cortex of Alzheimer's disease patients when analyzed by ANCOVA, (p=0.002). Therapeutic modulation of this gene, expressed protein and/or use of antibodies or small molecule drugs targeting the gene or gene product are useful in prevention or slowing the progression of Alzheimer's disease.

General_screening_panel_v1.4 Summary: Ag3562 Highest expression of this gene was detected in lung cancer A549 cell line (CT=20.01). High expression of this gene was also seen in cluster of cancer cell lines derived from gastric, colon, lung, renal, breast, ovarian, prostate, squamous cell carcinoma, melanoma and brain cancers. Thus, expression of this gene is useful as a marker to detect the presence of these cancers. Therapeutic modulation of this gene, expressed protein and/or use of antibodies or small molecule drugs targeting the gene or gene product are useful in the treatment of gastric, colon, lung, renal, breast, ovarian, prostate, squamous cell carcinoma, melanoma and brain cancers.

Among tissues with metabolic or endocrine function, this gene was expressed at moderate to high levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product are useful in the treatment of endocrine/metabolically related diseases, such as obesity and diabetes.

In addition, this gene was expressed at high levels in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia and depression.

HASS Panel v1.0 Summary: Ag3562. The expression of this gene was not increased by oxygen deprivation, acidic or a serum starved environment in the breast, bladder, pancreatic and prostate cell line in this panel.

However expression was increased in a glioblastoma/astrocytoma cell line when these cells are subjected to an acidic environment (Maximum expression U87-MG F11; CT=23.96) which indicates that expression may also be upregulated in the acidic regions of brain cancers. Moderate to low-expression was also shown in 2 of 5 glioma and 2 of 4 medulloblastoma tissue samples in this panel. Therapeutic modulation of this gene, expressed protein and/or use of antibodies or small molecule drugs targeting the gene or gene product are useful in the treatment of brain cancer.

Oncology_cell_line_screening_panel_v3.1 Summary: Ag3562 Highest expression of this gene was detected in lung carcinoid sample (CT=21.7). High to moderate levels of expression of this gene was also seen in number of cancer samples including tongue, breast, prostate, melanoma, bone marrow, bladder, pancreatic, renal, lymphoma, ovarian, cervical, uterine, gastric, lung and brain cancer. Therapeutic modulation of this gene, expressed protein and/or use of antibodies or small molecule drugs targeting the gene or gene product are useful in the treatment of cancers, including tongue, breast, prostate, melanoma, bone marrow, bladder, pancreatic, renal, lymphoma, ovarian, cervical, uterine, gastric, lung and brain cancer.

Panel 2D Summary: Ag3562 Highest expression of this gene was detected in lung cancer (CT=23.5). High expression of this gene was seen in number o f lung cancer samples. Expression of this gene was higher in cancer sample as compared to corresponding adjacent control samples. Therefore, expression of this gene is useful as marker to detect the presence of lung cancer. Therapeutic modulation of this gene, expressed protein and/or use of antibodies or small molecule drugs targeting the gene or gene product are useful in the treatment of lung cancer.

High to moderate levels of expression of this gene was also seen in number of cancer samples including colon, gastric, ovarian, liver, breast, thyroid, kidney, and prostate cancers. Therapeutic modulation of this gene, expressed protein and/or use of antibodies or small molecule drugs targeting the gene or gene product are useful in the treatment of these cancers.

Panel 4.1D Summary: Ag3562 Highest expression of this gene was detected in IL-4 treated dermal fibroblasts (CT=25.2). This gene was expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression indicates that this gene product may be involved in homeostatic processes for these and other cell types and tissues. This pattern is in agreement with the expression profile in General_screening_panel_v1.5 and also indicates a role for the gene product in cell survival and proliferation. Therapeutic modulation of this gene, expressed protein and/or use of antibodies or small molecule drugs targeting the gene or gene product are useful in the treatment of autoimmune and inflammatory diseases such as asthma, allergies, inflammatory bowel disease, lupus erythematosus, psoriasis, rheumatoid arthritis, and osteoarthritis.

Panel 5 Islet Summary: Ag3562 Highest expression of this gene was detected in islet cells (Bayer patient 1) (CT=25.3). High to moderate levels of expression of this gene were also seen in adipose, skeletal muscle, placenta, uterus, liver, heart, small intestine and kidney. Therefore, therapeutic modulation of the activity of this gene may prove useful in the treatment of endocrine/metabolically related diseases, such as obesity and diabetes.

BN. CG59839-02: Cation-Transporting Atpase.

Expression of gene CG59839-02 was assessed using the primer-probe sets Ag1417, Ag3604 and Ag3956, described in Tables BNA, BNB and BNC. Results of the RTQ-PCR runs are shown in Tables BND, BNE and BNF.

TABLE BNA
Probe Name Ag1417

| | | | | SEQ |
|---|---|---|---|---|
| | | | Start | ID |
| Primers | Sequences | Length | Position | No |
| Forward | 5′-ataggaaaatggacgcctacat-3′ | 22 | 2698 | 1440 |
| Probe | TET-5′-ccattgccggtctctgtaaacctgaa-3′- | 26 | 2737 | 1441 |
| | TAMRA | | | |
| Reverse | 5′-ttttgaaaatcgacaggaactg-3′ | 22 | 2764 | 1442 |

TABLE BNC
Probe Name Ag3956

| | | | | SEQ |
|---|---|---|---|---|
| | | | Start | ID |
| Primers | Sequences | Length | Position | No |
| Forward | 5′-cagcttgttcgttccatattgt-3 | 22 | 1956 | 1446 |
| Probe | TET-5′-tcccaaaccaactgattttaaactctaca- | 29 | 1979 | 1447 |
| | 3′-TAMRA | | | |
| Reverse | 5′-agcaactgccacaagacatagt-3′ | 22 | 2027 | 1448 |

TABLE BND
General_screening_panel v1.4
Column A - Rel. Exp. (%) Ag3604, Run 217674539
Column B - Rel. Exp. (%) Ag3956, Run 213856332

| Tissue Name | A | B | Tissue Name | A | B |
|---|---|---|---|---|---|
| Adipose | 5.6 | 9.2 | Renal ca. TK-10 | 17.9 | 28.5 |
| Melanoma* Hs688(A).T | 17.9 | 29.1 | Bladder | 10.9 | 14.4 |
| Melanoma* Hs688(B).T | 24.0 | 37.1 | Gastric ca. (liver met.) NCI-N87 | 17.0 | 22.4 |
| Melanoma* M14 | 12.3 | 21.9 | Gastric ca. KATO III | 38.7 | 55.9 |
| Melanoma* LOXIMVI | 13.4 | 22.1 | Colon ca. SW-948 | 4.4 | 6.9 |
| Melanoma* SK-MEL-5 | 17.8 | 24.1 | Colon ca. SW480 | 31.9 | 46.3 |
| Squamous cell carcinoma | 11.9 | 21.0 | Colon ca.* (SW480 met) SW620 | 17.0 | 25.3 |
| SCC-4 | | | | | |
| Testis Pool | 1.3 | 2.1 | Colon ca. HT29 | 9.1 | 14.1 |
| Prostate ca.* (bone met) PC-3 | 15.5 | 22.8 | Colon ca. HCT-116 | 27.9 | 45.1 |
| Prostate Pool | 1.4 | 2.1 | Colon ca. CaCo-2 | 14.8 | 22.8 |
| Placenta | 0.9 | 1.0 | Colon cancer tissue | 10.2 | 13.6 |
| Uterus Pool | 1.4 | 3.2 | Colon ca. SW1116 | 1.5 | 1.7 |
| Ovarian ca. OVCAR-3 | 12.4 | 20.9 | Colon ca. Colo-205 | 4.1 | 6.7 |
| Ovarian ca. SK-OV-3 | 24.3 | 35.6 | Colon ca. SW-48 | 5.8 | 4.3 |
| Ovarian ca. OVCAR-4 | 10.8 | 17.7 | Colon Pool | 4.0 | 7.7 |
| Ovarian ca. OVCAR-5 | 50.3 | 52.1 | Small Intestine Pool | 2.5 | 4.3 |
| Ovarian ca. IGROV-1 | 9.0 | 11.4 | Stomach Pool | 3.0 | 5.2 |
| Ovarian ca. OVCAR-8 | 5.4 | 5.8 | Bone Marrow Pool | 1.2 | 2.7 |
| Ovary | 2.1 | 4.9 | Fetal Heart | 5.6 | 7.3 |
| Breast ca. MCF-7 | 12.0 | 16.2 | Heart Pool | 2.1 | 2.8 |
| Breast ca. MDA-MB-231 | 15.3 | 23.2 | Lymph Node Pool | 4.7 | 7.5 |
| Breast ca. BT 549 | 9.2 | 14.7 | Fetal Skeletal Muscle | 0.6 | 1.0 |
| Breast ca. T47D | 100.0 | 100.0 | Skeletal Muscle Pool | 1.7 | 2.4 |
| Breast ca. MDA-N | 15.2 | 16.6 | Spleen Pool | 4.8 | 4.8 |
| Breast Pool | 3.9 | 7.9 | Thymus Pool | 2.9 | 5.4 |
| Trachea | 3.0 | 6.4 | CNS cancer (glio/astro) U87-MG | 84.7 | 98.6 |
| Lung | 0.5 | 0.8 | CNS cancer (glio/astro) U-118- | 30.8 | 51.4 |
| | | | MG | | |
| Fetal Lung | 8.0 | 10.6 | CNS cancer (neuro; met) SK-N- | 14.5 | 22.1 |
| | | | AS | | |
| Lung ca. NCI-N417 | 1.5 | 1.9 | CNS cancer (astro) SF-539 | 13.1 | 18.6 |
| Lung ca. LX-1 | 10.9 | 15.3 | CNS cancer (astro) SNB-75 | 39.8 | 50.0 |
| Lung ca. NCI-H146 | 11.7 | 20.0 | CNS cancer (glio) SNB-19 | 9.8 | 9.5 |
| Lung ca. SHP-77 | 5.3 | 8.1 | CNS cancer (glio) SF-295 | 30.6 | 43.8 |
| Lung ca. A549 | 9.6 | 15.3 | Brain (Amygdala) Pool | 1.9 | 2.7 |
| Lung ca. NCI-H526 | 4.5 | 5.3 | Brain (cerebellum) | 1.4 | 1.8 |
| Lung ca. NCI-H23 | 25.7 | 40.6 | Brain (fetal) | 4.4 | 7.4 |
| Lung ca. NCI-H460 | 5.9 | 7.2 | Brain (Hippocampus) Pool | 2.1 | 2.9 |
| Lung ca. HOP-62 | 5.8 | 7.0 | Cerebral Cortex Pool | 2.7 | 3.8 |
| Lung ca. NCI-H522 | 8.8 | 13.3 | Brain (Substantia nigra) Pool | 1.9 | 2.4 |
| Liver | 0.6 | 0.9 | Brain (Thalamus) Pool | 2.8 | 3.8 |
| Fetal Liver | 11.1 | 14.5 | Brain (whole) | 2.4 | 3.4 |
| Liver ca. HepG2 | 6.2 | 10.5 | Spinal Cord Pool | 1.9 | 2.1 |
| Kidney Pool | 5.2 | 10.8 | Adrenal Gland | 2.5 | 3.8 |
| Fetal Kidney | 4.2 | 6.4 | Pituitary gland Pool | 0.7 | 0.9 |
| Renal ca. 786-0 | 44.1 | 56.3 | Salivary Gland | 0.8 | 1.1 |
| Renal ca. A498 | 10.2 | 13.3 | Thyroid (female) | 5.0 | 7.5 |
| Renal ca. ACHN | 6.4 | 11.4 | Pancreatic ca. CAPAN2 | 12.0 | 18.4 |
| Renal ca. UO-31 | 37.9 | 49.0 | Pancreas Pool | 5.6 | 7.8 |

TABLE BNE
Panel 4.1D
Column A - Rel. Exp. (%) Ag3604, Run 169910577
Column B - Rel. Exp. (%) Ag3956, Run 170729090

| Tissue Name | A | B | Tissue Name | A | B |
|---|---|---|---|---|---|
| Secondary Th1 act | 14.2 | 11.5 | HUVEC IL-1 beta | 8.5 | 5.0 |
| Secondary Th2 act | 18.0 | 13.5 | HUVEC IFN gamma | 5.2 | 4.1 |
| Secondary Tr1 act | 17.9 | 10.2 | HUVEC TNF alpha + IFN gamma | 7.4 | 4.6 |
| Secondary Th1 rest | 1.6 | 1.1 | HUVEC TNF alpha + IL4 | 11.3 | 6.8 |
| Secondary Th2 rest | 3.8 | 2.7 | HUVEC IL-11 | 1.8 | 1.5 |
| Secondary Tr1 rest | 2.5 | 1.8 | Lung Microvascular EC none | 8.0 | 5.8 |
| Primary Th1 act | 11.8 | 9.0 | Lung Microvascular EC TNF alpha + | 24.1 | 17.0 |
| | | | IL-1 beta | | |
| Primary Th2 act | 13.6 | 10.2 | Microvascular Dermal EC none | 4.1 | 2.6 |
| Primary Tr1 act | 12.1 | 8.8 | Microsvasular Dermal EC TNF alpha + | 12.2 | 6.7 |
| | | | IL-1 beta | | |
| Primary Th1 rest | 3.6 | 2.0 | Bronchial epithelium TNF alpha + | 11.7 | 7.7 |
| | | | IL1 beta | | |
| Primary Th2 rest | 3.4 | 1.2 | Small airway epithelium none | 4.2 | 2.5 |
| Primary Tr1 rest | 3.4 | 3.0 | Small airway epithelium TNF alpha + | 13.6 | 9.3 |
| | | | IL-1 beta | | |
| CD45RA CD4 lymphocyte | 13.5 | 9.2 | Coronery artery SMC rest | 37.1 | 24.7 |
| act | | | | | |
| CD45RO CD4 lymphocyte | 14.8 | 10.4 | Coronery artery SMC TNF alpha + | 48.6 | 31.6 |
| act | | | IL-1 beta | | |
| CD8 lymphocyte act | 14.1 | 8.7 | Astrocytes rest | 6.7 | 3.7 |
| Secondary CD8 lymphocyte | 11.9 | 9.3 | Astrocytes TNF alpha + IL-1 beta | 15.1 | 7.9 |
| rest | | | | | |
| Secondary CD8 lymphocyte | 7.2 | 5.1 | KU-812 (Basophil) rest | 9.3 | 6.5 |
| act | | | | | |
| CD4 lymphocyte none | 1.6 | 1.2 | KU-812 (Basophil) PMA/ionomycin | 23.0 | 17.1 |
| 2ry Th1/Th2/Tr1 anti-CD95 | 2.8 | 2.5 | CCD1106 (Keratinocytes) none | 10.6 | 7.6 |
| CH11 | | | | | |
| LAK cells rest | 15.7 | 15.3 | CCD1106 (Keratinocytes) TNF alpha + | 16.2 | 10.1 |
| | | | IL-1 beta | | |
| LAK cells IL-2 | 6.7 | 5.3 | Liver cirrhosis | 3.5 | 1.8 |
| LAK cells IL-2 + IL-12 | 7.2 | 4.5 | NCI-H292 none | 6.0 | 4.0 |
| LAK cells IL-2 + IFN | 10.4 | 4.3 | NCI-H292 IL-4 | 13.3 | 7.4 |
| gamma | | | | | |
| LAK cells IL-2 + IL-18 | 9.4 | 4.9 | NCI-H292 IL-9 | 13.6 | 8.3 |
| LAK cells PMA/ionomycin | 60.7 | 34.2 | NCI-H292 IL-13 | 12.5 | 8.6 |
| NK Cells IL-2 rest | 7.2 | 5.0 | NCI-H292 IFN gamma | 13.7 | 8.1 |
| Two Way MLR 3 day | 15.1 | 7.0 | HPAEC none | 5.3 | 6.9 |
| Two Way MLR 5 day | 13.1 | 8.5 | HPAEC TNF alpha + IL-1 beta | 54.7 | 38.7 |
| Two Way MLR 7 day | 8.7 | 6.3 | Lung fibroblast none | 11.1 | 9.4 |
| PBMC rest | 1.6 | 1.2 | Lung fibroblast TNF alpha + IL-1 | 7.4 | 7.5 |
| | | | beta | | |
| PBMC PWM | 12.8 | 7.5 | Lung fibroblast IL-4 | 18.6 | 10.2 |
| PBMC PHA-L | 10.1 | 6.1 | Lung fibroblast IL-9 | 24.7 | 19.1 |
| Ramos (B cell) none | 10.0 | 5.0 | Lung fibroblast IL-13 | 13.8 | 10.2 |
| Ramos (B cell) ionomycin | 8.4 | 5.1 | Lung fibroblast IFN gamma | 20.4 | 14.6 |
| B lymphocytes PWM | 9.7 | 6.5 | Dermal fibroblast CCD1070 rest | 11.8 | 10.6 |
| B lymphocytes CD40L and | 6.7 | 3.8 | Dermal fibroblast CCD1070 TNF | 23.2 | 16.7 |
| IL-4 | | | alpha | | |
| EOL-1 dbcAMP | 7.9 | 5.1 | Dermal fibroblast CCD1070 IL-1 | 25.7 | 13.3 |
| | | | beta | | |
| EOL-1 dbcAMP | 24.0 | 16.0 | Dermal fibroblast IFN gamma | 12.2 | 8.4 |
| PMA/ionomycin | | | | | |
| Dendritic cells none | 23.3 | 13.4 | Dermal fibroblast IL-4 | 12.6 | 8.5 |
| Dendritic cells LPS | 28.7 | 20.7 | Dermal Fibroblast rest | 8.7 | 8.6 |
| Dendritic cells anti-CD40 | 18.6 | 12.9 | Neutrophils TNFa + LPS | 7.5 | 6.4 |
| Monocytes rest | 2.8 | 1.8 | Neutrophils rest | 0.6 | 0.7 |
| Monocytes LPS | 100.0 | 100.0 | Colon | 1.6 | 1.0 |
| Macrophages rest | 27.7 | 27.4 | Lung | 3.7 | 3.3 |
| Macrophages LPS | 24.8 | 15.5 | Thymus | 5.7 | 3.5 |
| HUVEC none | 3.5 | 2.3 | Kidney | 6.6 | 4.6 |
| HUVEC starved | 4.2 | 2.8 | | | |

TABLE BKE
general oncology screening panel_v_2.4
Column A - Rel. Exp. (%) Ag3540, Run 267294323

| Tissue Name | A | Tissue Name | A |
|---|---|---|---|
| Colon cancer 1 | 39.2 | Bladder NAT 2 | 0.2 |
| CC Margin (ODO3921) | 10.2 | Bladder NAT 3 | 0.3 |
| Colon cancer 2 | 45.4 | Bladder NAT 4 | 3.4 |
| Colon NAT 2 | 18.2 | Prostate adenocarcinoma 1 | 23.2 |
| Colon cancer 3 | 71.7 | Prostate adenocarcinoma 2 | 2.8 |
| Colon NAT 3 | 28.9 | Prostate adenocarcinoma 3 | 15.5 |
| Colon malignant cancer 4 | 92.0 | Prostate adenocarcinoma 4 | 33.4 |
| Colon NAT 4 | 10.4 | Prostate NAT 5 | 2.3 |
| Lung cancer 1 | 15.3 | Prostate adenocarcinoma 6 | 4.2 |
| Lung NAT 1 | 2.1 | Prostate adenocarcinoma 7 | 6.5 |
| Lung cancer 2 | 100.0 | Prostate adenocarcinoma 8 | 2.0 |
| Lung NAT 2 | 4.5 | Prostate adenocarcinoma 9 | 20.3 |
| Squamous cell carcinoma 3 | 91.4 | Prostate NAT 10 | 0.8 |
| Lung NAT 3 | 1.2 | Kidney cancer 1 | 60.3 |
| Metastatic melanoma 1 | 21.8 | Kidney NAT 1 | 13.6 |
| Melanoma 2 | 1.8 | Kidney cancer 2 | 71.2 |
| Melanoma 3 | 1.8 | Kidney NAT 2 | 26.1 |
| Metastatic melanoma 4 | 72.2 | Kidney cancer 3 | 55.5 |
| Metastatic melanoma 5 | 84.7 | Kidney NAT 3 | 8.7 |
| Bladder cancer 1 | 1.4 | Kidney cancer 4 | 54.7 |
| Bladder NAT 1 | 0.0 | Kidney NAT 4 | 17.6 |
| Bladder cancer 2 | 5.0 | | |

General_screening_panel_v1.4 Summary: Ag3604/Ag3956 Highest expression of this gene was seen in a breast cancer cell line (CTs=24-25). High levels of expression were also seen in all the cell lines on this panel. Significant levels of expression were seen in the fetal tissue samples. Expression in fetal liver and lung (CTs=27) was significantly higher than in the adult liver and lung (CTs=31.5). Furthermore, this expression profile indicates a role for this gene product in cell growth and proliferation.

Among tissues with metabolic function, this gene was expressed at moderate to low levels in pituitary, adipose, adrenal gland, pancreas, thyroid, and adult and fetal skeletal muscle, heart, and liver. This widespread expression among these tissues indicates that this gene product plays a role in normal neuroendocrine and metabolic tissues. Therapeutic modulation of this gene, expressed protein and/or use of antibodies or small molecule drugs targeting the gene or gene product are useful in the treatment of neuroendocrine disorders or metabolic diseases, such as obesity and diabetes.

This gene was also expressed at moderate levels in the CNS, including the hippocampus, thalamus, substantia nigra, amygdala, cerebellum and cerebral cortex. Therapeutic modulation of this gene, expressed protein and/or use of antibodies or small molecule drugs targeting the gene or gene product are useful in the treatment of neurologic disorders, such as Alzheimer's disease, Parkinson's disease, schizophrenia, multiple sclerosis, stroke and epilepsy.

The CG94820-02 gene codes for a cation-transporting ATPase A, P type. A P-type cation transporting ATPase has been implicated in Menkes disease, a disorder of copper transport characterized by progressive neurological degeneration and death in early childhood (Harrison M D, Dameron C T. (1999) Molecular mechanisms of copper metabolism and the role of the Menkes disease protein. J Biochem Mol Toxicol 1999; 13(2):93-106). Thus, the CG94820-02 gene product may play a role in this disease. Therapeutic modulation of this gene, expressed protein and/or use of antibodies or small molecule drugs targeting the gene or gene product are useful in the treatment of Menkes disease.

Panel 4.1D Summary: Ag3604/Ag3956 Highest expression of the CG94820-02 gene was seen in LPS stimulated monocytes (CTs=25-26). The protein encoded by this gene may therefore be involved in the activation of monocytes in their function as antigen-presenting cells. This indicates that therapeutics that block the function of this membrane protein are useful as anti-inflammatory therapeutics for the treatment of autoimmune and inflammatory diseases. Antibodies or small molecule therapeutics that stimulate the function of this protein may be useful therapeutics for the treatment of immunosupressed individuals.

This gene was expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression indicates that this gene product is involved in homeostatic processes for these and other cell types and tissues. This pattern is in agreement with the expression profile in General_screening_panel_v1.4 and also indicates a role for the gene product in cell survival and proliferation. Therapeutic modulation of this gene, expressed protein and/or use of antibodies or small molecule drugs targeting the gene or gene product are useful in the treatment of autoimmune and inflammatory diseases such as asthma, allergies, inflammatory bowel disease, lupus erythematosus, psoriasis, rheumatoid arthritis, and osteoarthritis.

general oncology screening panel_v_{—}2.4 Summary: Ag3604 Highest expression was detected in a lung cancer sample, with prominent expression seen in prostate and melanoma cancer samples. This gene was more highly expressed in lung, kidney, and colon cancers than in the normal adjacent tissues. Expression of this gene is useful as a marker of these cancers. Therapeutic modulation of this gene, expressed protein and/or use of antibodies or small molecule drugs targeting the gene or gene product are useful in the treatment of lung, colon, kidney, melanoma and prostate cancers.

BO. CG90866-03 and CG90866-04: Serine/Threonine-Protein Kinase.

Expression of genes CG90866-03 and CG90866-04 was assessed using the primer-probe sets Ag1088, Ag941 and Ag3771, described in Tables BOA, BOB and BOC. Results of the RTQ-PCR runs are shown in Tables BOD, BOE, BOF and BOG.

TABLE BOA
Probe Name Ag1088

| | | | | SEQ |
|---|---|---|---|---|
| | | | Start | ID |
| Primers | Sequences | Length | Position | No |
| Forward | 5′-cttgatgaagaaagcagaggaa-3′ | 22 | 776 | 1449 |
| Probe | TET-5′-atccagatcaaccaaggctcaccatt-3′- | 26 | 814 | 1450 |
| | TAMRA | | | |
| Reverse | 5′-agtcaggggcaatctgagatat-3′ | 22 | 843 | 1451 |

TABLE BOB
Probe Name Ag941

| | | | | SEQ |
|---|---|---|---|---|
| | | | Start | ID |
| Primers | Sequences | Length | Position | No |
| Forward | 5′-cctccactcagccatgatta-3′ | 20 | 1241 | 1452 |
| Probe | TET-5′-ataccgagacctgaaaccccacaatg-3′- | 26 | 1262 | 1453 |
| | TAMRA | | | |
| Reverse | 5′-gcagcattgggatacagtgt-3′ | 20 | 1299 | 1454 |

TABLE BOG
Probe Name Ag3771

| | | | | SEQ |
|---|---|---|---|---|
| | | | Start | ID |
| Primers | Sequences | Length | Position | No |
| Forward | 5′-ggcacaaagattttctcctttt-3 | 22 | 2247 | 1455 |
| Probe | TET-5′-tgatttcaccattcagaaactcattga-3′- | 27 | 2273 | 1456 |
| | TAMRA | | | |
| Reverse | 5′-gaaaacagttggcttgttcttg-3′ | 22 | 2302 | 1457 |

TABLE BOD
AI_comprehensive panel_v1.0
Column A - Rel. Exp. (%) Ag3771, Run 311756509

| Tissue Name | A | Tissue Name | A |
|---|---|---|---|
| 110967 COPD-F | 6.6 | 1112427 Match Control Psoriasis-F | 39.5 |
| 110980 COPD-F | 12.2 | 112418 Psoriasis-M | 5.7 |
| 110968 COPD-M | 9.1 | 112723 Match Control Psoriasis-M | 8.1 |
| 110977 COPD-M | 32.3 | 1112419 Psoriasis-M | 12.2 |
| 110989 Emphysema-F | 17.8 | 1112424 Match Control Psoriasis-M | 9.8 |
| 110992 Emphysema-F | 4.4 | 112420 Psoriasis-M | 23.0 |
| 110993 Emphysema-F | 8.5 | 112425 Match Control Psoriasis-M | 36.3 |
| 110994 Emphysema-F | 4.9 | 104689 (ME) OA Bone-Backus | 10.8 |
| 110995 Emphysema-F | 7.4 | 104690 (ME) Adj “Normal” Bone- | 9.7 |
| | | Backus | |
| 110996 Emphysema-F | 1.0 | 104691 (ME) OA Synovium-Backus | 17.7 |
| 110997 Asthma-M | 3.1 | 104692 (BA) OA Cartilage-Backus | 0.0 |
| 111001 Asthma-F | 10.7 | 104694 (BA) OA Bone-Backus | 8.5 |
| 111002 Asthma-F | 15.1 | 104695 (BA) Adj “Normal” Bone- | 9.9 |
| | | Backus | |
| 111003 Atopic Asthma-F | 10.5 | 104696 (BA) OA Synovium-Backus | 14.4 |
| 111004 Atopic Asthma-F | 8.0 | 104700 (55) OA Bone-Backus | 20.0 |
| 111005 Atopic Asthma-F | 9.5 | 104701 (SS) Adj “Normal” Bone-Backus | 9.3 |
| 111006 Atopic Asthma-F | 1.4 | 104702 (SS) OA Synovium-Backus | 22.5 |
| 111417 Allergy-M | 7.7 | 117093 OA Cartilage Rep7 | 7.7 |
| 112347 Allergy-M | 0.1 | 112672 OA Bone5 | 18.6 |
| 112349 Normal Lung-F | 0.1 | 112673 OA Synovium5 | 10.0 |
| 112357 Normal Lung-F | 20.2 | 1112674 OA Synovial Fluid Cells5 | 11.0 |
| 112354 Normal Lung-M | 11.4 | 117100 OA Cartilage Rep14 | 1.3 |
| 112374 Crohns-F | 5.1 | 112756 OA Bone9 | 1.6 |
| 112389 Match Control Crohns-F | 6.4 | 112757 OA Synovium9 | 22.7 |
| 112375 Crohns-F | 3.0 | 112758 OA Synovial Fluid Cells9 | 7.5 |
| 112732 Match Control Crohns-F | 8.4 | 117125 RA Cartilage Rep2 | 2.7 |
| 112725 Crohns-M | 2.4 | 113492 Bone2 RA | 85.3 |
| 112387 Match Control Crohns-M | 2.4 | 113493 Synovium2 RA | 26.4 |
| 112378 Crohns-M | 0.2 | 113494 Syn Fluid Cells RA | 48.0 |
| 112390 Match Control Crohns-M | 16.8 | 113499 Cartilage4 RA | 75.3 |
| 112726 Crohns-M | 12.4 | 113500 Bone4 RA | 100.0 |
| 112731 Match Control Crohns-M | 12.9 | 113501 Synovium RA | 85.9 |
| 112380 Ulcer Col-F | 11.5 | 113502 Syn Fluid Cells4 RA | 54.7 |
| 112734 Match Control Ulcer Col-F | 20.3 | 113495 Cartilage3 RA | 60.3 |
| 112384 Ulcer Col-F | 12.9 | 113496 Bone3 RA | 70.2 |
| 112737 Match Control Ulcer Col-F | 4.4 | 113497 Synovium3 RA | 45.4 |
| 112386 Ulcer Col-F | 5.4 | 113498 Syn Fluid Cells3 RA | 92.7 |
| 112738 Match Control Ulcer Col-F | 2.3 | 117106 Normal Cartilage Rep20 | 0.5 |
| 112381 Ulcer Col-M | 0.2 | 113663 Bone3 Normal | 0.0 |
| 112735 Match Control Ulcer Col- | 1.2 | 113664 Synovium3 Normal | 0.0 |
| M | | | |
| 112382 Ulcer Col-M | 8.5 | 113665 Syn Fluid Cells3 Normal | 0.1 |
| 112394 Match Control Ulcer Col- | 2.8 | 117107 Normal Cartilage Rep22 | 4.0 |
| M | | | |
| 112383 Ulcer Col-M | 3.6 | 113667 Bone4 Normal | 10.0 |
| 112736 13667 Match Control Ulcer | 4.0 | 113668 Synovium4 Normal | 9.3 |
| Col-M | | | |
| 112423 Psoriasis-F | 11.1 | 113669 Syn Fluid Cells4 Normal | 14.4 |

TABLE BOE
General_screening_panel_v1.4
Column A - Rel. Exp. (%) Ag3771, Run 218982528

| Tissue Name | A |
|---|---|
| Adipose | 11.7 |
| Melanoma* Hs688(A).T | 2.3 |
| Melanoma* Hs688(B).T | 0.9 |
| Melanoma* M14 | 23.0 |
| Melanoma* LOXIMVI | 0.6 |
| Melanoma* SK-MEL-5 | 23.7 |
| Squamous cell carcinoma SCC-4 | 0.0 |
| Testis Pool | 3.8 |
| Prostate ca.* (bone met) PC-3 | 1.3 |
| Prostate Pool | 4.3 |
| Placenta | 0.2 |
| Uterus Pool | 7.4 |
| Ovarian ca. OVCAR-3 | 0.3 |
| Ovarian ca. SK-OV-3 | 3.8 |
| Ovarian ca. OVCAR-4 | 0.0 |
| Ovarian ca. OVCAR-5 | 1.7 |
| Ovarian ca. IGROV-1 | 0.1 |
| Ovarian ca. OVCAR-8 | 0.1 |
| Ovary | 5.5 |
| Breast ca. MCF-7 | 0.0 |
| Breast ca. MDA-MB-231 | 0.1 |
| Breast ca. BT 549 | 0.0 |
| Breast ca. T47D | 5.0 |
| Breast ca. MDA-N | 4.5 |
| Breast Pool | 13.9 |
| Trachea | 5.3 |
| Lung | 5.0 |
| Fetal Lung | 100.0 |
| Lung ca. NCI-N417 | 0.2 |
| Lung ca. LX-1 | 0.0 |
| Lung ca. NCI-H146 | 0.0 |
| Lung ca. SHP-77 | 0.1 |
| Lung ca. A549 | 21.3 |
| Lung ca. NCI-H526 | 0.0 |
| Lung ca. NCI-H23 | 1.9 |
| Lung ca. NCI-H460 | 0.7 |
| Lung ca. HOP-62 | 0.4 |
| Lung ca. NCI-H522 | 0.0 |
| Liver | 0.3 |
| Fetal Liver | 9.3 |
| Liver ca. HepG2 | 0.0 |
| Kidney Pool | 23.2 |
| Fetal Kidney | 27.7 |
| Renal ca. 786-0 | 17.9 |
| Renal ca. A498 | 4.8 |
| Renal ca. ACHN | 9.0 |
| Renal ca. UO-31 | 4.0 |
| Renal ca. TK-10 | 5.6 |
| Bladder | 8.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.1 |
| Colon ca. CaCo-2 | 0.2 |
| Colon cancer tissue | 4.6 |
| Colon ca. SW1116 | 0.0 |
| Colon ca. Colo-205 | 0.0 |
| Colon ca. SW-48 | 0.0 |
| Colon Pool | 15.6 |
| Small Intestine Pool | 13.3 |
| Stomach Pool | 8.5 |
| Bone Marrow Pool | 5.9 |
| Fetal Heart | 2.0 |
| Heart Pool | 6.7 |
| Lymph Node Pool | 12.8 |
| Fetal Skeletal Muscle | 2.0 |
| Skeletal Muscle Pool | 5.9 |
| Spleen Pool | 16.6 |
| Thymus Pool | 7.2 |
| CNS cancer (glio/astro) | 4.7 |
| U87-MG | |
| CNS cancer (glio/astro) | 11.7 |
| U-118-MG | |
| CNS cancer (neuro; met) | 0.6 |
| SK-N-AS | |
| CNS cancer (astro) SF-539 | 0.1 |
| CNS cancer (astro) SNB-75 | 0.0 |
| CNS cancer (glio) SNB-19 | 0.5 |
| CNS cancer (glio) SF-295 | 3.1 |
| Brain (Amygdala) Pool | 4.9 |
| Brain (cerebellum) | 1.1 |
| Brain (fetal) | 2.9 |
| Brain (Hippocampus) Pool | 6.2 |
| Cerebral Cortex Pool | 12.5 |
| Brain (Substantia nigra) Pool | 7.6 |
| Brain (Thalamus) Pool | 13.8 |
| Brain (whole) | 5.7 |
| Spinal Cord Pool | 6.3 |
| Adrenal Gland | 3.7 |
| Pituitary gland Pool | 2.0 |
| Salivary Gland | 1.3 |
| Thyroid (female) | 7.7 |
| Pancreatic ca. CAPAN2 | 0.0 |
| Pancreas Pool | 9.7 |

TABLE BOF
Panel 4.1D
Column A - Rel. Exp. (%) Ag3771, Run 170130259
Column B - Rel. Exp. (%) Ag3771, Run 311582828

| Tissue Name | A | B | Tissue Name | A | B |
|---|---|---|---|---|---|
| Secondary Th1 act | 0.0 | 0.0 | HUVEC IL-1 beta | 0.1 | 0.0 |
| Secondary Th2 act | 0.0 | 0.0 | HUVEC IFN gamma | 0.7 | 0.5 |
| Secondary Tr1 act | 0.0 | 0.0 | HUVEC TNF alpha + IFN gamma | 0.1 | 0.0 |
| Secondary Th1 rest | 0.0 | 0.0 | HUVEC TNF alpha + IL4 | 0.0 | 0.0 |
| Secondary Th2 rest | 0.0 | 0.0 | HUVEC IL-11 | 0.2 | 0.1 |
| Secondary Tr1 rest | 0.0 | 0.0 | Lung Microvascular EC none | 0.0 | 0.1 |
| Primary Th1 act | 0.0 | 0.0 | Lung Microvascular EC TNF alpha + | 0.0 | 0.0 |
| | | | IL-1 beta | | |
| Primary Th2 act | 0.0 | 0.0 | Microvascular Dermal EC none | 0.0 | 0.0 |
| Primary Tr1 act | 0.0 | 0.0 | Microsvasular Dermal EC TNF alpha + | 0.0 | 0.0 |
| | | | IL-1 beta | | |
| Primary Th1 rest | 0.0 | 0.0 | Bronchial epithelium TNF alpha + | 0.3 | 0.1 |
| | | | IL1 beta | | |
| Primary Th2 rest | 0.0 | 0.0 | Small airway epithelium none | 0.1 | 0.0 |
| Primary Tr1 rest | 0.0 | 0.0 | Small airway epithelium TNF alpha + | 0.0 | 0.0 |
| | | | IL-1 beta | | |
| CD45RA CD4 lymphocyte | 0.6 | 0.7 | Coronery artery SMC rest | 1.0 | 0.9 |
| act | | | | | |
| CD45RO CD4 lymphocyte | 0.2 | 0.3 | Coronery artery SMC TNF alpha + | 0.8 | 0.7 |
| act | | | IL-1 beta | | |
| CD8 lymphocyte act | 0.1 | 0.0 | Astrocytes rest | 0.1 | 0.0 |
| Secondary CD8 lymphocyte | 0.0 | 0.0 | Astrocytes TNF alpha + IL-1 beta | 0.0 | 0.0 |
| rest | | | | | |
| Secondary CD8 lymphocyte | 0.0 | 0.0 | KU-812 (Basophil) rest | 0.0 | 0.0 |
| act | | | | | |
| CD4 lymphocyte none | 0.7 | 0.1 | KU-812 (Basophil) PMA/ionomycin | 0.1 | 0.0 |
| 2ry Th1/Th2/Tr1 anti-CD95 | 0.0 | 0.0 | CCD1106 (Keratinocytes) none | 0.0 | 0.0 |
| CH11 | | | | | |
| LAK cells rest | 25.9 | 5.6 | CCD1106 (Keratinocytes) TNF alpha + | 0.0 | 0.0 |
| | | | IL-1 beta | | |
| LAK cells IL-2 | 0.7 | 0.3 | Liver cirrhosis | 3.2 | 0.4 |
| LAK cells IL-2 + IL-12 | 0.6 | 0.1 | NCI-H292 none | 1.9 | 0.5 |
| LAK cells IL-2 + IFN | 1.3 | 0.2 | NCI-H292 IL-4 | 1.5 | 0.8 |
| gamma | | | | | |
| LAK cells IL-2 + IL-18 | 0.8 | 0.2 | NCI-H292 IL-9 | 2.1 | 1.8 |
| LAK cells PMA/ionomycin | 7.3 | 4.6 | NCI-H292 IL-13 | 1.3 | 1.0 |
| NK Cells IL-2 rest | 0.7 | 0.6 | NCI-H292 IFN gamma | 2.5 | 0.8 |
| Two Way MLR 3 day | 23.0 | 7.9 | HPAEC none | 0.8 | 0.3 |
| Two Way MLR 5 day | 7.7 | 0.0 | HPAEC TNF alpha + IL-1 beta | 0.7 | 0.4 |
| Two Way MLR 7 day | 1.7 | 0.2 | Lung fibroblast none | 1.4 | 1.0 |
| PBMC rest | 10.0 | 1.6 | Lung fibroblast TNF alpha + IL-1 | 3.9 | 4.2 |
| | | | beta | | |
| PBMC PWM | 2.0 | 0.1 | Lung fibroblast IL-4 | 0.5 | 0.3 |
| PBMC PHA-L | 3.0 | 1.1 | Lung fibroblast IL-9 | 1.2 | 0.3 |
| Ramos (B cell) none | 0.2 | 0.0 | Lung fibroblast IL-13 | 0.4 | 0.2 |
| Ramos (B cell) ionomycin | 0.1 | 0.0 | Lung fibroblast IFN gamma | 0.9 | 0.7 |
| B lymphocytes PWM | 1.6 | 0.8 | Dermal fibroblast CCD1070 rest | 0.5 | 0.3 |
| B lymphocytes CD40L and | 6.6 | 4.7 | Dermal fibroblast CCD1070 TNF | 0.4 | 0.3 |
| IL-4 | | | alpha | | |
| EOL-1 dbcAMP | 0.1 | 0.1 | Dermal fibroblast CCD1070 IL-1 | 0.7 | 0.5 |
| | | | beta | | |
| EOL-1 dbcAMP | 0.0 | 0.0 | Dermal fibroblast IFN gamma | 2.2 | 1.6 |
| PMA/ionomycin | | | | | |
| Dendritic cells none | 11.1 | 2.4 | Dermal fibroblast IL-4 | 1.6 | 0.8 |
| Dendritic cells LPS | 10.5 | 2.0 | Dermal Fibroblasts rest | 2.0 | 1.3 |
| Dendritic cells anti-CD40 | 8.1 | 1.5 | Neutrophils TNFa + LPS | 21.8 | 16.2 |
| Monocytes rest | 63.7 | 17.3 | Neutrophils rest | 100.0 | 100.0 |
| Monocytes LPS | 3.5 | 1.6 | Colon | 1.4 | 0.3 |
| Macrophages rest | 6.1 | 1.3 | Lung | 27.9 | 3.9 |
| Macrophages LPS | 6.6 | 2.9 | Thymus | 3.1 | 0.4 |
| HUVEC none | 0.2 | 0.0 | Kidney | 14.2 | 8.8 |
| HUVEC starved | 0.3 | 0.3 | | | |

TABLE BOG
general oncology screening panel_v_2.4
Column A - Rel. Exp. (%) Ag3771, Run 267820396
Column B - Rel. Exp. (%) Ag941, Run 262229106

| Tissue Name | A | B | Tissue Name | A | B |
|---|---|---|---|---|---|
| Colon cancer 1 | 0.8 | 1.1 | Bladder NAT 2 | 0.2 | 0.2 |
| CC Margin (ODO3921) | 0.9 | 1.4 | Bladder NAT 3 | 0.2 | 0.1 |
| Colon cancer 2 | 1.1 | 1.3 | Bladder NAT 4 | 1.6 | 2.3 |
| Colon NAT 2 | 0.7 | 0.9 | Prostate adenocarcinoma 1 | 13.4 | 14.9 |
| Colon cancer 3 | 1.8 | 1.8 | Prostate adenocarcinoma 2 | 0.6 | 0.5 |
| Colon NAT 3 | 2.6 | 2.9 | Prostate adenocarcinoma 3 | 2.0 | 2.8 |
| Colon malignant cancer 4 | 1.9 | 2.9 | Prostate adenocarcinoma 4 | 1.0 | 2.2 |
| Colon NAT 4 | 0.6 | 0.5 | Prostate NAT 5 | 0.3 | 0.2 |
| Lung cancer 1 | 5.8 | 6.2 | Prostate adenocarcinoma 6 | 0.3 | 0.2 |
| Lung NAT 1 | 5.1 | 8.4 | Prostate adenocarcinoma 7 | 0.7 | 0.9 |
| Lung cancer 2 | 20.3 | 23.0 | Prostate adenocarcinoma 8 | 0.4 | 0.4 |
| Lung NAT 2 | 14.8 | 17.3 | Prostate adenocarcinoma 9 | 4.3 | 4.0 |
| Squamous cell carcinoma 3 | 21.2 | 26.8 | Prostate NAT 10 | 0.2 | 0.2 |
| Lung NAT 3 | 5.6 | 0.8 | Kidney cancer 1 | 25.3 | 24.5 |
| Metastatic melanoma 1 | 7.2 | 6.6 | Kidney NAT 1 | 9.6 | 8.5 |
| Melanoma 2 | 0.1 | 0.1 | Kidney cancer 2 | 100.0 | 100.0 |
| Melanoma 3 | 0.4 | 0.5 | Kidney NAT 2 | 10.7 | 10.4 |
| Metastatic melanoma 4 | 12.4 | 12.2 | Kidney cancer 3 | 51.8 | 36.6 |
| Metastatic melanoma 5 | 11.0 | 12.1 | Kidney NAT 3 | 3.0 | 4.6 |
| Bladder cancer 1 | 0.6 | 0.7 | Kidney cancer 4 | 5.5 | 11.0 |
| Bladder NAT 1 | 0.0 | 0.0 | Kidney NAT 4 | 3.8 | 6.1 |
| Bladder cancer 2 | 1.4 | 1.1 | | | |

AI_comprehensive panel_v1.0 Summary: Ag3771 Highest expression of this gene was detected in a bone sample from a rheumatoid arthritis patient (CT=26). Prominent expression was detected in a cluster of rheumatoid arthritis samples, including samples from bone, synovium, and cartilage. Targeting this gene or gene product with small molecule, antibody, or protein therapeutics is useful in the treatment of rheumatoid arthritis.

General_screening_panel_v1.4 Summary: Ag3771 Highest expression of this gene was detected in fetal lung sample (CT=27.5). The expression of this gene is much higher in fetal (27-31) as compared to adult lung and liver (CT=32-35). Therefore, expression of this gene can be used to distinguish these fetal from adult tissues. In addition, the relative overexpression of this gene in these fetal tissues indicates that the protein product enhances growth or development of these tissues in the fetus and thus may also act in a regenerative capacity in the adult. Therapeutic modulation of this gene, expressed protein and/or use of antibodies or small molecule drugs targeting the gene or gene product are useful in the treatment of lung and liver related diseases.

Among tissues with metabolic or endocrine function, this gene was expressed at moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product are useful in the treatment of endocrine/metabolically related diseases, such as obesity and diabetes.

In addition, this gene was expressed at moderate levels in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia and depression.

Panel 4.1D Summary: Ag3771 Highest expression of this gene was detected in resting neutropils (CT=27.3). In addition, this gene was expressed in TNFalpha+LPS treated neutrophils. Therefore, the gene product may reduce activation of these inflammatory cells and be useful as a protein therapeutic to reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. Therapeutic modulation of this gene, expressed protein and/or use of antibodies or small molecule drugs targeting the gene or gene product are useful in increasing the immune response in patients with AIDS or other immunodeficiencies.

In addition, expression of this gene was down-regulated in cytokine stimulated LAK cells and LPS-treated monocytes. Therefore, expression of this gene is useful for distinguishing these stimulated versus resting cells.

In addition, low to moderate expression of this gene was also seen in B cells, dendritic cells, endothelial cells, fibroblasts and normal tissues represented by kidney, thymus, lung, and colon. Therapeutic modulation of this gene, expressed protein and/or use of antibodies or small molecule drugs targeting the gene or gene product are useful in the treatment of cancer, Crohn's disease, ulcerative colitis, multiple sclerosis, chronic obstructive pulmonary disease, asthma, emphysema, rheumatoid arthritis, lupus erythematosus, or psoriasis, microbial and viral infections.

general oncology screening panel_v_{—}2.4 Summary: Ag941/Ag3771 Highest expression of this gene was detected in a kidney cancer sample (CTs=27). Prominent expression was also seen in prostate and melanoma cancer samples. This gene was overexpressed in the kidney cancer samples when compared to expression in the normal adjacent tissue. Expression of this gene is useful as a marker of kidney cancer. Therapeutic modulation of this gene, expressed protein and/or use of antibodies or small molecule drugs targeting the gene or gene product are useful in the treatment of kidney, melanoma and prostate cancers.

BP. CG9170802: Stromelysin-1.

Expression of gene CG91708-02 was assessed using the primer-probe set Ag3395, described in Table BPA. Results of the RTQ-PCR runs are shown in Tables BPB, BPC, BPD, BPE, BPF, BPG, BPH, BPI and BPJ. CG91708-02 represents a full-length physical clone of the CG91708-01 gene.

TABLE BPA
Probe Name Ag3395

| | | | | SEQ |
|---|---|---|---|---|
| | | | Start | ID |
| Primers | Sequences | Length | Position | No |
| Forward | 5′-gtaaagccagtggaaatgaaga-3′ | 22 | 36 | 1458 |
| Probe | TET-5′-tcttccaatcctactgttgctgtgcg-3- | 26 | 59 | 1459 |
| | TAMRA | | | |
| Reverse | 5′-caatggataggctgagcaaac-3′ | 21 | 90 | 1460 |

TABLE BPB
AI.05 chondrosarcoma
Column A - Rel. Exp. (%) Ag3395, Run 306941365

| Tissue Name | A |
|---|---|
| 138353 PMA (18 hrs) | 1.2 |
| 138352 IL-1beta + Oncostatin M (18 hrs) | 37.4 |
| 138351 IL-1beta + TNFa (18 hrs) | 100.0 |
| 138350 IL-1beta (18 hrs) | 31.9 |
| 138354 Untreated-complete medium (18 hrs) | 0.2 |
| 138347 PMA (6 hrs) | 4.0 |
| 138346 IL-1beta + Oncostatin M (6 hrs) | 29.5 |
| 138345 IL-1beta + TNFa (6 hrs) | 20.6 |
| 138344 IL-1beta (6 hrs) | 8.4 |
| 138348 Untreated-complete medium (6 hrs) | 0.9 |
| 138349 Untreated-serum starved (6 hrs) | 1.8 |

TABLE BPC
AI_comprehensive panel_v1.0
Column A - Rel. Exp. (%) Ag3395, Run 217700657

| Tissue Name | A | Tissue Name | A |
|---|---|---|---|
| 110967 COPD-F | 0.0 | 1112427 Match Control Psoriasis-F | 0.0 |
| 110980 COPD-F | 0.0 | 112418 Psoriasis-M | 0.0 |
| 110968 COPD-M | 0.0 | 112723 Match Control Psoriasis-M | 0.0 |
| 110977 COPD-M | 0.0 | 1112419 Psoriasis-M | 0.0 |
| 110989 Emphysema-F | 0.0 | 1112424 Match Control Psoriasis-M | 0.0 |
| 110992 Emphysema-F | 0.0 | 112420 Psoriasis-M | 0.0 |
| 110993 Emphysema-F | 0.0 | 112425 Match Control Psoriasis-M | 0.0 |
| 110994 Emphysema-F | 0.0 | 104689 (ME) OA Bone-Backus | 1.0 |
| 110995 Emphysema-F | 0.0 | 104690 (ME) Adj “Normal” Bone- | 2.3 |
| | | Backus | |
| 110996 Emphysema-F | 0.0 | 104691 (ME) OA Synovium-Backus | 4.9 |
| 110997 Asthma-M | 0.0 | 104692 (BA) OA Cartilage-Backus | 27.9 |
| 111001 Asthma-F | 0.0 | 104694 (BA) OA Bone-Backus | 2.6 |
| 111002 Asthma-F | 0.0 | 104695 (BA) Adj “Normal” Bone- | 90.1 |
| | | Backus | |
| 111003 Atopic Asthma-F | 0.0 | 104696 (BA) OA Synovium-Backus | 100.0 |
| 111004 Atopic Asthma-F | 0.0 | 104700 (SS) OA Bone-Backus | 0.7 |
| 111005 Atopic Asthma-F | 0.0 | 104701 (SS) Adj “Normal” Bone-Backus | 14.1 |
| 111006 Atopic Asthma-F | 0.0 | 104702 (SS) OA Synovium-Backus | 1.6 |
| 111417 Allergy-M | 0.0 | 117093 OA Cartilage Rep7 | 0.3 |
| 112347 Allergy-M | 0.0 | 112672 OA Bone5 | 0.6 |
| 112349 Normal Lung-F | 0.0 | 112673 OA Synovium5 | 0.3 |
| 112357 Normal Lung-F | 0.0 | 1112674 OA Synovial Fluid Cells5 | 0.3 |
| 112354 Normal Lung-M | 0.0 | 117100 OA Cartilage Rep14 | 0.0 |
| 112374 Crohns-F | 0.0 | 112756 OA Bone9 | 0.0 |
| 112389 Match Control Crohns-F | 0.1 | 112757 OA Synovium9 | 0.0 |
| 112375 Crohns-F | 0.0 | 112758 OA Synovial Fluid Cells9 | 0.0 |
| 112732 Match Control Crohns-F | 0.0 | 117125 RA Cartilage Rep2 | 0.0 |
| 112725 Crohns-M | 0.1 | 113492 Bone2 RA | 0.0 |
| 112387 Match Control Crohns-M | 0.2 | 113493 Synovium2 RA | 0.0 |
| 112378 Crohns-M | 0.0 | 113494 Syn Fluid Cells RA | 0.0 |
| 112390 Match Control Crohns-M | 0.0 | 113499 Cartilage4 RA | 0.0 |
| 112726 Crohns-M | 0.0 | 113500 Bone4 RA | 0.0 |
| 112731 Match Control Crohns-M | 0.0 | 113501 Synovium RA | 0.0 |
| 112380 Ulcer Col-F | 0.0 | 113502 Syn Fluid Cells4 RA | 0.0 |
| 112734 Match Control Ulcer Col-F | 0.3 | 113495 Cartilage3 RA | 0.0 |
| 112384 Ulcer Col-F | 0.0 | 113496 Bone3 RA | 0.0 |
| 112737 Match Control Ulcer Col-F | 0.0 | 113497 Synovium3 RA | 0.0 |
| 112386 Ulcer Col-F | 0.3 | 113498 Syn Fluid Cells3 RA | 0.1 |
| 112738 Match Control Ulcer Col-F | 3.0 | 117106 Normal Cartilage Rep20 | 0.0 |
| 112381 Ulcer Col-M | 0.0 | 113663 Bone3 Normal | 0.0 |
| 112735 Match Control Ulcer Col- | 0.2 | 113664 Synovium3 Normal | 0.0 |
| M | | | |
| 112382 Ulcer Col-M | 0.0 | 113665 Syn Fluid Cells3 Normal | 0.0 |
| 112394 Match Control Ulcer Col- | 0.1 | 117107 Normal Cartilage Rep22 | 0.0 |
| M | | | |
| 112736 13667 Match Control Ulcer | 0.0 | 113668 Synovium4 Normal | 0.0 |
| Col-M | | | |
| 112383 Ulcer Col-M | 0.0 | 113667 Bone4 Normal | 0.0 |
| 112423 Psoriasis-F | 0.0 | 113669 Syn Fluid Cells4 Normal | 0.0 |

TABLE BPD
Ardais Panel v.1.0
Column A - Rel. Exp. (%) Ag3395, Run 263151265

| Tissue Name | A |
|---|---|
| 136799 Lung cancer(362) | 0.8 |
| 136800 Lung NAT(363) | 0.4 |
| 136813 Lung cancer(372) | 77.9 |
| 136814 Lung NAT(373) | 0.2 |
| 136815 Lung cancer(374) | 1.1 |
| 136816 Lung NAT(375) | 100.0 |
| 136791 Lung cancer(35A) | 0.5 |
| 136795 Lung cancer(35E) | 9.9 |
| 136797 Lung cancer(360) | 0.1 |
| 136794 lung NAT(35D) | 0.1 |
| 136818 Lung NAT(377) | 0.3 |
| 136787 lung cancer(356) | 0.1 |
| 136788 lung NAT(357) | 0.1 |
| 136804 Lung cancer(369) | 0.6 |
| 136805 Lung NAT(36A) | 0.1 |
| 136806 Lung cancer(36B) | 4.3 |
| 136807 Lung NAT(36C) | 0.1 |
| 136789 lung cancer(358) | 0.0 |
| 136802 Lung cancer(365) | 0.2 |
| 136803 Lung cancer(368) | 0.2 |
| 136811 Lung cancer(370) | 31.2 |
| 136810 Lung NAT(36F) | 0.9 |

TABLE BPE
Ardais Prostate 1.0
Column A - Rel. Exp. (%) Ag3395, Run 320416133

| Tissue Name | A | Tissue Name | A |
|---|---|---|---|
| 151135 Prostate NAT(B87) | 1.7 | 151128 Prostate cancer(B8C) | 34.4 |
| 151143 Prostate NAT(B8A) | 7.9 | 151136 Prostate cancer(B8B) | 34.4 |
| 153669 Prostate NAT(D5E) | 5.8 | 151144 Prostate cancer(B8F) | 3.4 |
| 153677 Prostate NAT(D66) | 0.8 | 153654 Prostate cancer(D4F) | 100.0 |
| 153685 Prostate NAT(D6E) | 5.1 | 153662 Prostate cancer(D57) | 2.8 |
| 145905 Prostate NAT(A0C) | 3.5 | 153655 Prostate cancer(D50) | 89.5 |
| 153670 Prostate NAT(D5F) | 5.3 | 145907 Prostate cancer(A0A) | 15.5 |
| 153678 Prostate NAT(D67) | 13.3 | 153663 Prostate cancer(D58) | 2.6 |
| 153686 Prostate NAT(D6F) | 6.0 | 151130 Prostate cancer(B90) | 69.3 |
| 145906 Prostate NAT(A09) | 15.4 | 153648 Prostate cancer(D49) | 2.8 |
| 151129 Prostate NAT(B93) | 4.0 | 153656 Prostate cancer(D51) | 5.8 |
| 151137 Prostate NAT(B86) | 3.7 | 153664 Prostate cancer(D59) | 0.1 |
| 153671 Prostate NAT(D60) | 10.4 | 155799 Prostate cancer(EA8) | 6.4 |
| 151145 Prostate NAT(B91) | 25.3 | 145909 Prostate cancer(9E7) | 23.8 |
| 153679 Prostate NAT(D68) | 2.7 | 153649 Prostate cancer(D4A) | 4.9 |
| 153687 Prostate NAT(D70) | 2.0 | 153657 Prostate cancer(D52) | 9.0 |
| 153672 Prostate NAT(D61) | 12.4 | 153665 Prostate cancer(D5A) | 5.5 |
| 153680 Prostate NAT(D69) | 5.9 | 151132 Prostate cancer(B88) | 23.8 |
| 151131 Prostate NAT(B85) | 1.8 | 153650 Prostate cancer(D4B) | 3.9 |
| 153673 Prostate NAT(D62) | 4.8 | 153658 Prostate cancer(D53) | 3.5 |
| 153681 Prostate NAT(D6A) | 1.3 | 153666 Prostate cancer(D5B) | 9.7 |
| 145910 Prostate NAT(9G3) | 0.9 | 153651 Prostate cancer(D4C) | 0.7 |
| 153674 Prostate NAT(D63) | 26.2 | 153659 Prostate cancer(D54) | 7.1 |
| 153682 Prostate NAT(D6B) | 10.7 | 153667 Prostate cancer(D5C) | 33.9 |
| 151133 Prostate NAT(B94) | 12.2 | 151134 Prostate cancer(B92) | 31.4 |
| 153675 Prostate NAT(D64) | 9.8 | 151142 Prostate cancer(B89) | 1.6 |
| 153683 Prostate NAT(D6C) | 15.7 | 153652 Prostate cancer(D4D) | 2.1 |
| 153668 Prostate NAT(D5D) | 34.9 | 153660 Prostate cancer(D55) | 3.4 |
| 153676 Prostate NAT(D65) | 6.4 | 149773 Prostate NAT(AD8) | 16.5 |
| 153684 Prostate NAT(D6D) | 2.7 | 149774 Prostate cancer(AD7) | 6.4 |
| 145904 Prostate cancer(9E2) | 2.3 | 151139 Prostate NAT(B8E) | 6.5 |
| 149776 Prostate cancer(AD5) | 49.3 | 151138 Prostate cancer(B8D) | 8.5 |
| 153653 Prostate cancer(D4E) | 5.4 | 151141 Prostate NAT(B96) | 2.8 |
| 153661 Prostate cancer(D56) | 4.2 | 151140 Prostate cancer(B95) | 4.1 |

TABLE BPF
General_screening_panel_v1.4
Column A - Rel. Exp. (%) Ag3395, Run 208034252
Column B - Rel. Exp. (%) Ag3395, Run 212141064

| Tissue Name | A | B | Tissue Name | A | B |
|---|---|---|---|---|---|
| Adipose | 0.1 | 0.1 | Renal ca. TK-10 | 0.0 | 0.0 |
| Melanoma* Hs688(A).T | 1.2 | 1.9 | Bladder | 0.1 | 0.1 |
| Melanoma* Hs688(B).T | 0.3 | 0.5 | Gastric ca. (liver met.) NCI-N87 | 0.5 | 0.8 |
| Melanoma* M14 | 0.1 | 0.1 | Gastric ca. KATO III | 0.4 | 0.8 |
| Melanoma* LOXIMVI | 3.2 | 6.6 | Colon ca. SW-948 | 0.0 | 0.0 |
| Melanoma* SK-MEL-5 | 0.0 | 0.0 | Colon ca. SW480 | 0.0 | 0.0 |
| Squamous cell carcinoma | 0.0 | 0.1 | Colon ca.* (SW480 met) SW620 | 0.0 | 0.0 |
| SCC-4 | | | | | |
| Testis Pool | 0.8 | 1.2 | Colon ca. HT29 | 0.0 | 0.0 |
| Prostate ca.* (bone met) PC-3 | 0.1 | 0.1 | Colon ca. HCT-116 | 0.0 | 0.0 |
| Prostate Pool | 0.1 | 0.2 | Colon ca. CaCo-2 | 0.1 | 0.1 |
| Placenta | 0.0 | 0.0 | Colon cancer tissue | 30.1 | 37.1 |
| Uterus Pool | 0.0 | 0.0 | Colon ca. SW1116 | 0.0 | 0.0 |
| Ovarian ca. OVCAR-3 | 0.0 | 0.1 | Colon ca. Colo-205 | 0.0 | 0.0 |
| Ovarian ca. SK-OV-3 | 0.0 | 0.3 | Colon ca. SW-48 | 0.0 | 0.0 |
| Ovarian ca. OVCAR-4 | 0.0 | 0.0 | Colon Pool | 0.0 | 0.0 |
| Ovarian ca. OVCAR-5 | 0.2 | 0.4 | Small Intestine Pool | 0.6 | 1.2 |
| Ovarian ca. IGROV-1 | 0.0 | 0.1 | Stomach Pool | 2.2 | 3.7 |
| Ovarian ca. OVCAR-8 | 0.0 | 0.0 | Bone Marrow Pool | 0.0 | 0.0 |
| Ovary | 0.0 | 0.0 | Fetal Heart | 0.0 | 0.0 |
| Breast ca. MCF-7 | 0.0 | 0.0 | Heart Pool | 0.0 | 0.0 |
| Breast ca. MDA-MB-231 | 0.0 | 0.0 | Lymph Node Pool | 0.0 | 0.0 |
| Breast ca. BT 549 | 0.1 | 0.2 | Fetal Skeletal Muscle | 0.0 | 0.0 |
| Breast ca. T47D | 0.1 | 0.3 | Skeletal Muscle Pool | 0.1 | 0.2 |
| Breast ca. MDA-N | 0.1 | 0.2 | Spleen Pool | 0.1 | 0.1 |
| Breast Pool | 0.1 | 0.3 | Thymus Pool | 0.0 | 0.1 |
| Trachea | 1.6 | 1.8 | CNS cancer (glio/astro) U87-MG | 100.0 | 100.0 |
| Lung | 0.0 | 0.0 | CNS cancer (glio/astro) U-118- | 52.5 | 72.7 |
| | | | MG | | |
| Fetal Lung | 0.1 | 0.1 | CNS cancer (neuro; met) SK-N-AS | 0.0 | 0.0 |
| Lung ca. NCI-N417 | 0.0 | 0.0 | CNS cancer (astro) SF-539 | 0.1 | 0.2 |
| Lung ca. LX-1 | 0.0 | 0.0 | CNS cancer (astro) SNB-75 | 0.3 | 0.7 |
| Lung ca. NCI-H146 | 0.0 | 0.0 | CNS cancer (glio) SNB-19 | 0.1 | 0.2 |
| Lung ca. SHP-77 | 0.0 | 0.0 | CNS cancer (glio) SF-295 | 21.2 | 54.0 |
| Lung ca. A549 | 0.0 | 0.0 | Brain (Amygdala) Pool | 0.0 | 0.0 |
| Lung ca. NCI-H526 | 0.0 | 0.0 | Brain (cerebellum) | 0.0 | 0.0 |
| Lung ca. NCI-H23 | 0.0 | 0.4 | Brain (fetal) | 0.0 | 0.0 |
| Lung ca. NCI-H460 | 0.0 | 0.2 | Brain (Hippocampus) Pool | 0.1 | 0.2 |
| Lung ca. HOP-62 | 0.0 | 0.0 | Cerebral Cortex Pool | 0.0 | 0.0 |
| Lung ca. NCI-H522 | 0.1 | 0.3 | Brain (Substantia nigra) Pool | 0.0 | 0.0 |
| Liver | 0.0 | 0.0 | Brain (Thalamus) Pool | 0.0 | 0.0 |
| Fetal Liver | 0.0 | 0.0 | Brain (whole) | 0.0 | 0.1 |
| Liver ca. HepG2 | 0.0 | 0.0 | Spinal Cord Pool | 0.0 | 0.0 |
| Kidney Pool | 0.0 | 0.0 | Adrenal Gland | 0.0 | 0.1 |
| Fetal Kidney | 0.3 | 0.5 | Pituitary gland Pool | 0.0 | 0.0 |
| Renal ca. 786-0 | 0.0 | 0.0 | Salivary Gland | 0.1 | 0.0 |
| Renal ca. A498 | 0.0 | 0.0 | Thyroid (female) | 0.0 | 0.0 |
| Renal ca. ACHN | 0.0 | 0.8 | Pancreatic ca. CAPAN2 | 0.1 | 0.1 |
| Renal ca. UO-31 | 0.0 | 0.0 | Pancreas Pool | 0.0 | 0.1 |

TABLE BPG
Panel 2D
Column A - Rel. Exp. (%) Ag3395, Run 165469036

| Tissue Name | A |
|---|---|
| Normal Colon | 4.4 |
| CC Well to Mod Diff (OD03866) | 48.6 |
| CC Margin (OD03866) | 4.6 |
| CC Gr.2 rectosigmoid (OD03868) | 9.0 |
| CC Margin (OD03868) | 0.3 |
| CC Mod Duff (OD03920) | 10.9 |
| CC Margin (OD03920) | 1.8 |
| CC Gr.2 ascend colon (OD03921) | 100.0 |
| CC Margin (OD03921) | 3.1 |
| CC from Partial Hepatectomy (OD04309) Mets | 1.4 |
| Liver Margin (OD04309) | 0.3 |
| Colon mets to lung (OD04451-01) | 0.1 |
| Lung Margin (OD04451-02) | 0.0 |
| Normal Prostate 6546-1 | 1.9 |
| Prostate cancer (OD04410) | 0.3 |
| Prostate Margin (OD04410) | 0.0 |
| Prostate cancer (OD04720-01) | 0.5 |
| Prostate Margin (OD04720-02) | 0.9 |
| Normal Lung | 0.4 |
| Lung Met to Muscle (OD04286) | 0.4 |
| Muscle Margin (OD04286) | 9.3 |
| Lung Malignant cancer (OD03126) | 2.6 |
| Lung Margin (OD03126) | 0.3 |
| Lung cancer (OD04404) | 25.9 |
| Lung Margin (OD04404) | 0.2 |
| Lung cancer (OD04565) | 21.9 |
| Lung Margin (OD04565) | 0.4 |
| Lung cancer (OD04237-01) | 1.4 |
| Lung Margin (OD04237-02) | 0.3 |
| Ocular Mel Met to Liver (OD04310) | 0.1 |
| Liver Margin (OD04310) | 0.1 |
| Melanoma Metastasis | 0.2 |
| Lung Margin (OD04321) | 0.3 |
| Normal Kidney | 1.7 |
| Kidney Ca, Nuclear grade 2 (OD04338) | 0.1 |
| Kidney Margin (OD04338) | 1.0 |
| Kidney Ca Nuclear grade 1/2 (OD04339) | 0.1 |
| Kidney Margin (OD04339) | 1.4 |
| Kidney Ca, Clear cell type (OD04340) | 0.0 |
| Kidney Margin (OD04340) | 0.5 |
| Kidney Ca, Nuclear grade 3 (OD04348) | 0.0 |
| Kidney Margin (OD04348) | 1.2 |
| Kidney cancer (OD04622-01) | 0.1 |
| Kidney Margin (OD04622-03) | 0.3 |
| Kidney cancer (OD04450-01) | 0.3 |
| Kidney Margin (OD04450-03) | 0.2 |
| Kidney cancer 8120607 | 0.5 |
| Kidney Margin 8120608 | 0.0 |
| Kidney cancer 8120613 | 0.0 |
| Kidney Margin 8120614 | 0.2 |
| Kidney cancer 9010320 | 0.6 |
| Kidney Margin 9010321 | 1.1 |
| Normal Uterus | 0.5 |
| Uterine cancer 064011 | 0.9 |
| Normal Thyroid | 0.2 |
| Thyroid cancer | 0.0 |
| Thyroid cancer A302152 | 0.9 |
| Thyroid Margin A302153 | 0.0 |
| Normal Breast | 5.8 |
| Breast cancer | 3.8 |
| Breast cancer (OD04590-01) | 2.7 |
| Breast cancer Mets (OD04590-03) | 2.5 |
| Breast cancer Metastasis | 0.3 |
| Breast cancer | 17.7 |
| Breast cancer | 4.1 |
| Breast cancer 9100266 | 18.2 |
| Breast Margin 9100265 | 30.4 |
| Breast cancer A209073 | 16.8 |
| Breast Margin A209073 | 19.3 |
| Normal Liver | 0.1 |
| Liver cancer | 0.1 |
| Liver cancer 1025 | 0.0 |
| Liver cancer 1026 | 0.0 |
| Liver cancer 6004-T | 0.0 |
| Liver Tissue 6004-N | 1.6 |
| Liver cancer 6005-T | 0.0 |
| Liver Tissue 6005-N | 0.0 |
| Normal Bladder | 0.5 |
| Bladder cancer | 0.6 |
| Bladder cancer | 4.3 |
| Bladder cancer (OD04718-01) | 13.4 |
| Bladder Normal Adjacent (OD04718-03) | 35.4 |
| Normal Ovary | 0.0 |
| Ovarian cancer | 1.3 |
| Ovarian cancer (OD04768-07) | 0.0 |
| Ovary Margin (OD04768-08) | 1.7 |
| Normal Stomach | 1.3 |
| Gastric cancer 9060358 | 6.9 |
| Stomach Margin 90603591 | 1.4 |
| Gastric cancer 90603951 | 10.2 |
| Stomach Margin 90603941 | 1.3 |
| Gastric cancer 9060397 | 25.0 |
| Stomach Margin 9060396 | 1.0 |
| Gastric cancer 064005 | 60.7 |

TABLE BPH
Panel 3D
Column A - Rel. Exp. (%) Ag3395, Run 165924467
Column B - Rel. Exp. (%) Ag3395, Run 167542915

| Tissue Name | A | B | Tissue Name | A | B |
|---|---|---|---|---|---|
| 94905 Daoy | 0.0 | 0.0 | 94954 Ca Ski Cervical | 0.3 | 0.2 |
| Medulloblastoma/Cerebellum | | | epidermoid carcinoma | | |
| | | | (metastasis | | |
| 94906 TE671 | 0.0 | 0.0 | 94955 ES-2 Ovarian clear cell | 3.1 | 4.0 |
| Medulloblastom/Cerebellum | | | carcinoma | | |
| 94907 D283 Med | 0.0 | 0.0 | 94957 Ramos Stimulated with | 0.0 | 0.0 |
| Medulloblastoma/Cerebellum | | | PMA/ionomycin 6 h | | |
| 94908 PFSK-1 Primitive | 0.0 | 0.0 | 94958 Ramos Stimulated with | 0.0 | 0.0 |
| Neuroectodermal/Cerebellum | | | PMA/ionomycin 14 h | | |
| 94909 XF-498 CNS | 0.1 | 0.1 | 94962 MEG-01 Chronic | 0.0 | 0.0 |
| | | | myelogenous leukemia | | |
| | | | (megokaryoblast) | | |
| 94910 SNB-78 CNS/glioma | 0.3 | 0.2 | 94963 Raji Burkitt's | 0.0 | 0.0 |
| | | | lymphoma | | |
| 94911 SF-268 CNS/glioblastoma | 0.0 | 0.1 | 94964 Daudi Burkitt's | 0.0 | 0.0 |
| | | | lymphoma | | |
| 94912 T98G Glioblastoma | 0.8 | 1.3 | 94965 U266 B-cell | 0.0 | 0.0 |
| | | | plasmacytoma/myeloma | | |
| 96776 SK-N-SH Neuroblastoma | 12.9 | 16.7 | 94968 CA46 Burkitt's | 0.0 | 0.0 |
| (metastasis) | | | lymphoma | | |
| 94913 SF-295 CNS/glioblastoma | 100.0 | 100.0 | 94970 RL non-Hodgkin's B- | 0.0 | 0.0 |
| | | | cell lymphoma | | |
| 94914 Cerebellum | 0.0 | 0.0 | 94972 JM1 pre-B-cell | 0.0 | 0.0 |
| | | | lymphoma/leukemia | | |
| 96777 Cerebellum | 0.0 | 0.0 | 94973 Jurkat T cell leukemia | 0.0 | 0.0 |
| 94916 NCI-H292 | 0.0 | 0.0 | 94974 TF-1 Erythroleukemia | 0.0 | 0.0 |
| Mucoepidermoid lung carcinoma | | | | | |
| 94917 DMS-114 Small cell lung | 0.2 | 0.3 | 94975 HUT 78 T-cell | 0.0 | 0.0 |
| cancer | | | lymphoma | | |
| 94918 DMS-79 Small cell lung | 0.0 | 0.0 | 94977 U937 Histiocytic | 0.0 | 0.0 |
| cancer/neuroendocrine | | | lymphoma | | |
| 94919 NCI-H146 Small cell lung | 0.0 | 0.0 | 94980 KU-812 Myelogenous | 0.0 | 0.2 |
| cancer/neuroendocrine | | | leukemia | | |
| 94920 NCI-H526 Small cell lung | 0.0 | 0.0 | 769-P- Clear cell renal | 0.0 | 0.0 |
| cancer/neuroendocrine | | | carcinoma | | |
| 94921 NCI-N417 Small cell lung | 0.0 | 0.0 | 94983 Caki-2 Clear cell renal | 0.0 | 0.0 |
| cancer/neuroendocrine | | | carcinoma | | |
| 94923 NCI-H82 Small cell lung | 0.0 | 0.0 | 94984 SW 839 Clear cell renal | 0.0 | 0.0 |
| cancer/neuroendocrine | | | carcinoma | | |
| 94924 NCI-H157 Squamous cell | 0.0 | 0.0 | 94986 G401 Wilms' tumor | 0.0 | 0.0 |
| lung cancer (metastasis) | | | | | |
| 94925 NCI-H1155 Large cell | 0.0 | 0.0 | 94987 Hs766T Pancreatic | 0.0 | 0.0 |
| lung cancer/neuroendocrine | | | carcinoma (LN metastasis) | | |
| 94926 NCI-H1299 Large cell | 0.0 | 0.0 | 94988 CAPAN-1 Pancreatic | 0.0 | 0.0 |
| lung cancer/neuroendocrine | | | adenocarcinoma (liver | | |
| | | | metastasis) | | |
| 94927 NCI-H727 Lung carcinoid | 0.0 | 0.0 | 94989 SU86.86 Pancreatic | 0.0 | 0.1 |
| | | | carcinoma (liver metastasis) | | |
| 94928 NCI-UMC-11 Lung | 0.1 | 0.0 | 94990 BxPC-3 Pancreatic | 0.2 | 0.1 |
| carcinoid | | | adenocarcinoma | | |
| 94929 LX-1 Small cell lung | 0.0 | 0.0 | 94991 HPAC Pancreatic | 0.0 | 0.0 |
| cancer | | | adenocarcinoma | | |
| 94930 Colo-205 Colon cancer | 0.0 | 0.0 | 94992 MIA PaCa-2 Pancreatic | 0.0 | 0.0 |
| | | | carcinoma | | |
| 94931 KM12 Colon cancer | 0.0 | 0.0 | 94993 CFPAC-1 Pancreatic | 0.0 | 0.1 |
| | | | ductal adenocarcinoma | | |
| 94932 KM20L2 Colon cancer | 0.0 | 0.0 | 94994 PANC-1 Pancreatic | 0.0 | 0.0 |
| | | | epithelioid ductal carcinoma | | |
| 94933 NCI-H716 Colon cancer | 0.0 | 0.0 | 94996 T24 Bladder carcinma | 0.0 | 0.0 |
| | | | (transitional cell | | |
| 94935 SW-48 Colon | 0.0 | 0.0 | 5637- Bladder carcinoma | 0.0 | 0.0 |
| adenocarcinoma | | | | | |
| 94936 SW1116 Colon | 0.0 | 0.0 | 94998 HT-1197 Bladder | 0.0 | 0.1 |
| adenocarcinoma | | | carcinoma | | |
| 94937 LS 174T Colon | 0.0 | 0.0 | 94999 UM-UC-3 Bladder | 0.2 | 0.2 |
| adenocarcinoma | | | carcinma (transitional cell) | | |
| 94938 SW-948 Colon | 0.0 | 0.0 | 95000 A204 | 0.2 | 0.2 |
| adenocarcinoma | | | Rhabdomyosarcoma | | |
| 94939 SW-480 Colon | 0.0 | 0.0 | 95001 HT-1080 Fibrosarcoma | 0.0 | 0.1 |
| adenocarcinoma | | | | | |
| 94940 NCI-SNU-5 Gastric | 0.0 | 0.0 | 95002 MG-63 Osteosarcoma | 0.3 | 0.5 |
| carcinoma | | | (bone) | | |
| KATO III- Gastric carcinoma | 0.0 | 0.0 | 95003 SK-LMS-1 | 23.7 | 38.4 |
| | | | Leiomyosarcoma (vulva) | | |
| 94943 NCI-SNU-16 Gastric | 0.1 | 0.2 | 95004 SJRH30 | 0.0 | 0.0 |
| carcinoma | | | Rhabdomyosarcoma (met to | | |
| | | | bone marrow) | | |
| 94944 NCI-SNU-1 Gastric | 0.0 | 0.0 | 95005 A431 Epidermoid | 0.0 | 0.0 |
| carcinoma | | | carcinoma | | |
| 94946 RF-1 Gastric | 0.0 | 0.0 | 95007 WM266-4 Melanoma | 0.0 | 0.0 |
| adenocarcinoma | | | | | |
| 94947 RF-48 Gastric | 0.0 | 0.0 | DU 145- Prostate carcinoma | 0.0 | 0.0 |
| adenocarcinoma | | | (brain metastasis) | | |
| 96778 MKN-45 Gastric | 0.0 | 0.0 | 95012 MDA-MB-468 Breast | 0.0 | 0.0 |
| carcinoma | | | adenocarcinoma | | |
| 94949 NCI-N87 Gastric | 0.0 | 0.0 | SCC-4- Squamous cell | 0.0 | 0.0 |
| carcinoma | | | carcinoma of tongue | | |
| 94951 OVCAR-5 Ovarian | 0.0 | 0.0 | SCC-9- Squamous cell | 0.0 | 0.0 |
| carcinoma | | | carcinoma of tongue | | |
| 94952 RL95-2 Uterine carcinoma | 0.0 | 0.0 | SCC-15- Squamous cell | 0.0 | 0.0 |
| | | | carcinoma of tongue | | |
| 94953 HelaS3 Cervical | 0.0 | 0.0 | 95017 CAL 27 Squamous cell | 0.0 | 4.3 |
| adenocarcinoma | | | carcinoma of tongue | | |

TABLE BPI
Panel 4D
Column A - Rel. Exp. (%) Ag3395, Run 166447040

| Tissue Name | A | Tissue Name | A |
|---|---|---|---|
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL-1 | 0.0 |
| | | beta | |
| Primary Th2 act | 0.0 | Microvascular Dermal EC none | 0.0 |
| Primary Tr1 act | 0.0 | Microsvasular Dermal EC TNF alpha + IL-1 | 0.0 |
| | | beta | |
| Primary Th1 rest | 0.0 | Bronchial epithelium TNF alpha + IL1beta | 4.0 |
| Primary Th2 rest | 0.0 | Small airway epithelium none | 0.8 |
| Primary Tr1 rest | 0.0 | Small airway epithelium TNF alpha + IL-1 | 7.7 |
| | | beta | |
| CD45RA CD4 lymphocyte act | 23.3 | Coronery artery SMC rest | 0.6 |
| CD45RO CD4 lymphocyte act | 0.0 | Coronery artery SMC TNF alpha + IL-1 beta | 0.8 |
| CD8 lymphocyte act | 0.0 | Astrocytes rest | 0.0 |
| Secondary CD8 lymphocyte rest | 0.0 | Astrocytes TNF alpha + IL-1 beta | 0.3 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 0.0 | CCD1106 (Keratinocytes) TNF alpha + IL-1 | 0.1 |
| | | beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.3 |
| PBMC PWM | 0.0 | Lung fibroblast TNF alpha + IL-1 beta | 56.6 |
| PBMC PHA-L | 0.0 | Lung fibroblast IL-4 | 0.2 |
| Ramos (B cell) none | 0.0 | Lung fibroblast IL-9 | 1.7 |
| Ramos (B cell) ionomycin | 0.0 | Lung fibroblast IL-13 | 0.1 |
| B lymphocytes PWM | 0.0 | Lung fibroblast IFN gamma | 0.2 |
| B lymphocytes CD40L and IL-4 | 0.0 | Dermal fibroblast CCD1070 rest | 16.5 |
| EOL-1 dbcAMP | 0.0 | Dermal fibroblast CCD1070 TNF alpha | 57.0 |
| EOL-1 dbcAMP | 0.0 | Dermal fibroblast CCD1070 IL-1 beta | 100.0 |
| PMA/ionomycin | 0.0 | | |
| Dendritic cells none | 0.0 | Dermal fibroblast IFN gamma | 1.7 |
| Dendritic cells LPS | 0.0 | Dermal fibroblast IL-4 | 2.9 |
| Dendritic cells anti-CD40 | 0.0 | IBD Colitis 2 | 0.1 |
| Monocytes rest | 0.0 | IBD Crohn's | 0.0 |
| Monocytes LPS | 0.0 | Colon | 0.1 |
| Macrophages rest | 0.0 | Lung | 0.0 |
| Macrophages LPS | 0.0 | Thymus | 0.1 |
| HUVEC none | 0.0 | Kidney | 0.0 |
| HUVEC starved | 0.0 | | |

TABLE BPJ
Panel 5 Islet
Column A - Rel. Exp. (%) Ag3395, Run 259154756

| Tissue Name | A | Tissue Name | A |
|---|---|---|---|
| 97457 Patient-02go adipose | 0.0 | 94709 Donor 2 AM - A adipose | 8.0 |
| 97476 Patient-07sk skeletal muscle | 15.3 | 94710 Donor 2 AM - B adipose | 4.1 |
| 97477 Patient-07ut uterus | 0.0 | 94711 Donor 2 AM - C adipose | 4.5 |
| 97478 Patient-07pl placenta | 0.0 | 94712 Donor 2 AD - A adipose | 0.3 |
| 99167 Bayer Patient 1 | 100.0 | 94713 Donor 2 AD - B adipose | 0.5 |
| 97482 Patient-08ut uterus | 2.3 | 94714 Donor 2 AD - C adipose | 1.0 |
| 97483 Patient-08pl placenta | 0.4 | 94742 Donor 3 U - A Mesenchymal | 0.6 |
| | | Stem Cells | |
| 97486 Patient-09sk skeletal muscle | 0.5 | 94743 Donor 3 U - B Mesenchymal | 0.3 |
| | | Stem Cells | |
| 97487 Patient-09ut uterus | 0.1 | 94730 Donor 3 AM - A adipose | 21.6 |
| 97488 Patient-09pl placenta | 0.0 | 94731 Donor 3 AM - B adipose | 12.7 |
| 97492 Patient-10ut uterus | 0.4 | 94732 Donor 3 AM - C adipose | 13.2 |
| 97493 Patient-10pl placenta | 0.0 | 94733 Donor 3 AD - A adipose | 2.9 |
| 97495 Patient-11go adipose | 0.0 | 94734 Donor 3 AD - B adipose | 3.0 |
| 97496 Patient-11sk skeletal muscle | 0.3 | 94735 Donor 3 AD - C adipose | 0.6 |
| 97497 Patient-11ut uterus | 1.0 | 77138 Liver HepG2untreated | 0.3 |
| 97498 Patient-11pl placenta | 0.0 | 73556 Heart Cardiac stromal cells | 0.3 |
| | | (primary) | |
| 97500 Patient-12go adipose | 0.0 | 81735 Small Intestine | 1.9 |
| 97501 Patient-12sk skeletal muscle | 0.0 | 72409 Kidney Proximal Convoluted | 0.3 |
| | | Tubule | |
| 97502 Patient-12ut uterus | 0.2 | 82685 Small intestine Duodenum | 1.8 |
| 97503 Patient-12p1 placenta | 0.3 | 90650 Adrenal Adrenocortical | 0.0 |
| | | adenoma | |
| 94721 Donor 2 U - A Mesenchymal | 0.3 | 72410 Kidney HRCE | 1.2 |
| Stem Cells | | | |
| 94722 Donor 2 U - B Mesenchymal | 0.7 | 72411 Kidney HRE | 0.3 |
| Stem Cells | | | |
| 94723 Donor 2 U - C Mesenchymal | 0.0 | 73139 Uterus Uterine smooth muscle | 22.1 |
| Stem Cells | | cells | |

AI.05 chondrosarcoma Summary: Ag3395 Highest expression of this gene was detected in IL-1 and TNF alpha treated chondrosarcoma cell line (SW1353) (CT=18.8). Expression of this gene was upregulated upon IL-1 treatment, a potent activator of pro-inflammatory cytokines and matrix metalloproteinases. This gene codes for matrix metalloproteinase 3 (MMP3), which is capable of degrading proteoglycan, fibronectin, laminin, and type IV collagen. MMPs are known to participate in the destruction of cartilage observed in Osteoarthritis (OA). Therapeutic modulation of this gene, expressed protein and/or use of antibodies or small molecule drugs targeting the gene or gene product are useful in the prevention of the degeneration of cartilage observed in OA.

AI_comprehensive panel_v1.0 Summary: Ag3395 This gene was expressed in orthoarthritis (OA) tissues but not in control tissue. This gene encodes MMP3 protein, which has been shown to be present in OA joint (Bluteau G, Conrozier T, Mathieu P, Vignon E, Herbage D, Mallein-Gerin F. Matrix metalloproteinase-1, -3, -13 and aggrecanase-1 and -2 are differentially expressed in experimental osteoarthritis. Biochim Biophys Acta 2001 May 3; 1526(2):147-58) tissue and may contribute to the pathology of this disease. Therapeutic modulation of this gene, expressed protein and/or use of antibodies or small molecule drugs targeting the gene or gene product are useful in the treatment of OA.

Ardais Panel v.1.0 Summary: Ag3395 Highest expression of this gene was detected in a normal adjacent lung (375) sample (CT=24.7). Significant expression of this gene was seen in normal and cancer samples from lung. This gene shows up-regulated expression in 4/6 cancer samples relative to corresponding normal adjacent samples. Therefore, modulation of this, expressed protein, and/or use of antibodies or small molecule drug targeting this gene or gene product will be of use to treat lung cancer.

Ardais Prostate 1.0 Summary: Ag3395 Highest expression of this gene was detected in prostate cancer (D4F) sample (CT=27). Significant expression of this gene was seen in normal and cancer samples from prostate. The expression of this gene was relatively higher in number of prostate cancer samples. Therefore, modulation of this, expressed protein, and/or use of antibodies or small molecule drug targeting this gene or gene product will be of use to treat prostate cancer.

General_screening_panel_v1.4 Summary: Ag3395 The expression of this gene was highest in a sample derived a brain cancer cell line (U87-MG) (CTs=22-24). Significant expression of this gene was also seen in brain cancer cell lines, colon cancer cell lines and melanoma cell lines. Modulation of this gene, encoded protein and/or use of antibodies or small molecule drug targeting this gene or gene product is useful in the treatment of brain or colon cancer or melanoma.

Among tissues with metabolic function, this gene was expressed at low levels in pancreas, adipose, and fetal skeletal muscle. This expression indicates that this gene product plays a role in normal neuroendocrine and metabolic and that disregulated expression of this gene will contribute to neuroendocrine disorders or metabolic diseases, such as obesity and diabetes.

This gene was also expressed at low but significant levels in the hippocampus, a structure critical for learning and memory. The hippocampus-preferential expression of this gene indicate that it plays a role in learning and memory processes. Modulation of this gene is useful in treatment of CNS disorders involving memory deficits, including Alzheimer's disease and aging.

Panel 2D Summary: Ag3395 Highest expression of this gene was detected in a sample derived from a colon cancer (CT=26.8). Significant expression was also seen in gastric cancer, bladder cancer, breast cancer, lung cancer and colon cancer. Expression levels of this gene is useful as marker to detect these cancers. Therapeutic modulation of this gene, encoded protein and/or use of antibodies or small molecule drug targeting this gene or gene product is useful in the treatment of gastric, bladder, breast, lung or colon cancers.

Panel 3D Summary: Ag3395 The expression of this gene was highest in a sample derived from a brain cancer cell line (SF-295) (CTs=24-26). Significant levels of expression of this gene was also seen in cell lines derived from brain, lung, ovarian, cervical, pancreatic, vulval, and bone cancers. Therapeutic modulation of this gene, encoded protein and/or use of antibodies or small molecule drugs is useful in the treatment of brain lung, ovarian, cervical, pancreatic, vulval, and bone cancers.

Panel 4D Summary: Ag3395 The expression level of this gene was up-regulated in lung and dermal fibroblasts after treatment with IL-1 beta and/or TNF alpha (CTs=21.5-22.5). High expression of this gene was also seen in activated small airway and bronchial epithelium, activated naive T cells (CD45RA CD4 lymphocyte), activated asatrocytes, resting and activated coronary artery SMC cells. This expression profile indicates that the stromolysin protein encoded by this gene may facilitate tissue destruction, remodeling and participate in cell:cell interactions that prevent the resolution of the inflammatory response. Modulation of this gene, encoded protein and/or use of antibodies or small molecule drug targeting this gene or gene product will help to reduce or eliminate inflammation in the skin and lung resulting from psoriasis, allergy, asthma, emphysema, promote wound healing and prevent delayed type hypersensitivity type reactions.

Panel 5 Islet Summary: Ag3395 Highest expression of this gene was detected in islet cells (bayer patient 1) (CT=27.9). Significant expression of this gene was also seen in adipose, skeletal muscle and uterus. Modulation of this gene or expressed protein is useful in the treatment of metabolic disorders, including type II diabetes and obesity.

BQ. CG94235-01: Thymidylate Kinase.

Expression of gene CG94235-01 was assessed using the primer-probe sets Ag1980 and Ag3909, described in Tables BQA and BQB. Results of the RTQ-PCR runs are shown in Tables BQC, BQD, BQE, BQF, BQG, BQH, BQI and BQJ.

TABLE BOA
Probe Name Ag1980

| | | | | SEQ |
|---|---|---|---|---|
| | | | Start | ID |
| Primers | Sequences | Length | Position | No |
| Forward | 5′-gggtacgatggctgaagtaaa-3′ | 21 | 1437 | 1461 |
| Probe | TET-5′-ccagttttctgccacacaacatgctt-3′- | 26 | 1394 | 1462 |
| | TAMRA | | | |
| Reverse | 5′-ttatgcagtgttcccaaatttc-3′ | 22 | 1364 | 1463 |

TABLE BOB
Probe Name Ag3909

| | | | | SEQ |
|---|---|---|---|---|
| | | | Start | ID |
| Primers | Sequences | Length | Position | No |
| Forward | 5′-caggtgccacgtctaactagat-3′ | 22 | 1307 | 1464 |
| Probe | TET-5′-tgttgtttgaaacatctacatccacca-3′- | 27 | 1333 | 1465 |
| | TAMRA | | | |
| Reverse | 5′-gaaatttgggaacactgcataa-3′ | 22 | 1364 | 1466 |

TABLE BQG
AI.05 chondrosarcoma
Column A - Rel. Exp.(%) Ag1980, Run 306913835

| Tissue Name | A | Tissue Name | A |
|---|---|---|---|
| 138353 PMA (18 hrs) | 0.3 | 138346 IL-1beta + Oncostatin M | 9.9 |
| | | (6 hrs) | |
| 138352 IL-1beta + Oncostatin M | 0.8 | 138345 IL-1beta + TNFa (6 hrs) | 19.2 |
| (18 hrs) | | | |
| 138351 IL-1beta + TNFa (18 hrs) | 100.0 | 138344 IL-1beta (6 hrs) | 7.5 |
| 138350 IL-1beta (18 hrs) | 13.7 | 138348 Untreated-complete medium | 0.1 |
| | | (6 hrs) | |
| 138354 Untreated-complete medium | 0.0 | 138349 Untreated-serum starved | 0.4 |
| (18 hrs) | | (6 hrs) | |
| 138347 PMA (6 hrs) | 0.4 | | |

TABLE BQD
AI_comprehensive panel_v1.0
Column A - Rel. Exp. (%) Ag1980, Run 211061884
Column B - Rel. Exp. (%) Ag1980, Run 212317511

| Tissue Name | A | B | Tissue Name | A | B |
|---|---|---|---|---|---|
| 110967 COPD-F | 10.1 | 11.6 | 112427 Match Control Psoriasis-F | 100.0 | 100.0 |
| 110980 COPD-F | 12.5 | 16.8 | 112418 Psoriasis-M | 19.1 | 18.7 |
| 110968 COPD-M | 13.9 | 13.5 | 112723 Match Control Psoriasis- | 6.6 | 7.2 |
| | | | M | | |
| 110977 COPD-M | 50.3 | 32.8 | 112419 Psoriasis-M | 35.6 | 17.0 |
| 110989 Emphysema-F | 20.7 | 24.5 | 112424 Match Control Psoriasis- | 7.6 | 10.1 |
| | | | M | | |
| 110992 Emphysema-F | 12.2 | 6.0 | 112420 Psoriasis-M | 54.0 | 53.2 |
| 110993 Emphysema-F | 12.9 | 8.7 | 112425 Match Control Psoriasis- | 62.4 | 72.7 |
| | | | M | | |
| 110994 Emphysema-F | 7.0 | 5.8 | 104689 (MF) OA Bone-Backus | 66.0 | 56.3 |
| 110995 Emphysema-F | 17.0 | 17.6 | 104690 (MF) Adj “Normal” Bone- | 34.6 | 24.0 |
| | | | Backus | | |
| 110996 Emphysema-F | 3.3 | 7.0 | 104691 (MF) OA Synovium- | 36.6 | 40.9 |
| | | | Backus | | |
| 110997 Asthma-M | 8.0 | 3.1 | 104692 (BA) OA Cartilage- | 12.1 | 6.8 |
| | | | Backus | | |
| 111001 Asthma-F | 21.8 | 15.8 | 104694 (BA) OA Bone-Backus | 51.8 | 36.3 |
| 111002 Asthma-F | 24.0 | 13.1 | 104695 (BA) Adj “Normal” Bone- | 26.4 | 19.5 |
| | | | Backus | | |
| 111003 Atopic Asthma-F | 20.3 | 22.5 | 104696 (BA) OA Synovium- | 63.7 | 40.9 |
| | | | Backus | | |
| 111004 Atopic Asthma-F | 26.1 | 28.5 | 104700 (SS) OA Bone-Backus | 20.9 | 62.0 |
| 111005 Atopic Asthma-F | 24.5 | 10.3 | 104701 (SS) Adj “Normal” Bone- | 28.7 | 25.7 |
| | | | Backus | | |
| 111006 Atopic Asthma-F | 11.0 | 8.1 | 104702 (SS) OA Synovium- | 80.1 | 59.5 |
| | | | Backus | | |
| 111417 Allergy-M | 8.7 | 7.7 | 117093 OA Cartilage Rep7 | 17.8 | 20.9 |
| 112347 Allergy-M | 0.0 | 0.0 | 112672 OA Bone5 | 36.6 | 33.9 |
| 112349 Normal Lung-F | 0.0 | 0.0 | 112673 OA Synovium5 | 21.3 | 22.4 |
| 112357 Normal Lung-F | 67.4 | 64.6 | 112674 OA Synovial Fluid cells5 | 20.3 | 20.0 |
| 112354 Normal Lung-M | 9.8 | 15.6 | 117100 OA Cartilage Rep14 | 9.6 | 6.9 |
| 112374 Crohns-F | 11.1 | 15.0 | 112756 OA Bone9 | 95.3 | 66.0 |
| 112389 Match Control | 10.4 | 18.4 | 112757 OA Synovium9 | 17.4 | 19.2 |
| Crohns-F | | | | | |
| 112375 Crohns-F | 14.5 | 9.9 | 112758 OA Synovial Fluid Cells9 | 14.1 | 17.8 |
| 112732 Match Control | 29.3 | 28.5 | 117125 RA Cartilage Rep2 | 19.9 | 22.5 |
| Crohns-F | | | | | |
| 112725 Crohns-M | 5.1 | 3.6 | 113492 Bone2 RA | 76.3 | 66.0 |
| 112387 Match Control | 14.4 | 15.7 | 113493 Synovium2 RA | 20.9 | 19.3 |
| Crohns-M | | | | | |
| 112378 Crohns-M | 0.0 | 0.0 | 113494 Syn Fluid Cells RA | 48.0 | 43.2 |
| 112390 Match Control | 33.7 | 48.6 | 113499 Cartilage4 RA | 40.3 | 49.7 |
| Crohns-M | | | | | |
| 112726 Crohns-M | 25.0 | 19.9 | 113500 Bone4 RA | 63.3 | 50.3 |
| 112731 Match Control | 19.1 | 16.8 | 113501 Synovium4 RA | 44.8 | 36.6 |
| Crohns-M | | | | | |
| 112380 Ulcer Col-F | 14.8 | 15.9 | 113502 Syn Fluid Cells4 RA | 33.7 | 21.5 |
| 112734 Match Control Ulcer | 74.2 | 59.9 | 113495 Cartilage3 RA | 48.3 | 29.3 |
| Col-F | | | | | |
| 112384 Ulcer Col-F | 26.2 | 28.9 | 113496 Bone3 RA | 51.8 | 45.1 |
| 112737 Match Control Ulcer | 8.8 | 4.1 | 113497 Synovium3 RA | 21.9 | 25.3 |
| Col-F | | | | | |
| 112386 Ulcer Col-F | 11.8 | 10.9 | 113498 Syn Fluid Cells3 RA | 44.1 | 47.6 |
| 112738 Match Control Ulcer | 27.0 | 15.0 | 117106 Normal Cartilage Rep20 | 2.0 | 5.3 |
| Col-F | | | | | |
| 112381 Ulcer Col-M | 0.0 | 0.0 | 113663 Bone3 Normal | 0.0 | 0.2 |
| 112735 Match Control Ulcer | 6.3 | 6.5 | 113664 Synovium3 Normal | 0.0 | 0.0 |
| Col-M | | | | | |
| 112382 Ulcer Col-M | 13.5 | 10.8 | 113665 Syn Fluid Cells3 Normal | 0.0 | 0.0 |
| 112394 Match Control Ulcer | 1.6 | 7.7 | 117107 Normal Cartilage Rep22 | 10.6 | 11.8 |
| Col-M | | | | | |
| 112383 Ulcer Col-M | 18.7 | 26.6 | 113667 Bone4 Normal | 10.1 | 8.4 |
| 112736 Match Control Ulcer | 12.9 | 6.7 | 113668 Synovium4 Normal | 10.6 | 8.1 |
| Col-M | | | | | |
| 112423 Psoriasis-F | 20.7 | 18.6 | 113669 Syn Fluid Cells4 Normal | 15.8 | 21.3 |

TABLE BQE
General_screening_panel_v1.4
Column A - Rel. Exp. (%) Ag3909, Run 217235826
Column B - Rel. Exp. (%) Ag3909, Run 219173644

| Tissue Name | A | B | Tissue Name | A | B |
|---|---|---|---|---|---|
| Adipose | 0.6 | 0.6 | Renal ca. TK-10 | 0.0 | 0.0 |
| Melanoma* Hs688(A).T | 0.0 | 0.0 | Bladder | 9.7 | 11.7 |
| Melanoma* Hs688(B).T | 0.0 | 0.0 | Gastric ca. (liver met.) NCI-N87 | 100.0 | 100.0 |
| Melanoma* M14 | 0.3 | 0.4 | Gastric ca. KATO III | 0.5 | 0.6 |
| Melanoma* LOXIMVI | 0.1 | 0.2 | Colon ca. SW-948 | 0.5 | 0.5 |
| Melanoma* SK-MEL-5 | 0.2 | 0.2 | Colon ca. SW480 | 0.1 | 0.1 |
| Squamous Cell carcinoma | 0.5 | 0.5 | Colon ca.* (SW480 met) SW620 | 0.0 | 0.0 |
| SCC-4 | | | | | |
| Testis Pool | 0.6 | 0.5 | Colon ca. HT29 | 0.1 | 0.1 |
| Prostate ca.* (bone met) PC-3 | 0.1 | 0.1 | Colon ca. HCT-116 | 0.3 | 0.3 |
| Prostate Pool | 0.3 | 0.3 | Colon ca. CaCo-2 | 0.1 | 0.0 |
| Placenta | 0.4 | 0.5 | Colon cancer tissue | 0.6 | 0.5 |
| Uterus Pool | 0.2 | 0.2 | Colon ca. SW1116 | 0.2 | 0.1 |
| Ovarian ca. OVCAR-3 | 0.2 | 0.2 | Colon ca. Colo-205 | 0.0 | 0.0 |
| Ovarian ca. SK-OV-3 | 0.5 | 0.4 | Colon ca. SW-48 | 0.0 | 0.0 |
| Ovarian ca. OVCAR-4 | 0.1 | 0.1 | Colon Pool | 0.4 | 0.5 |
| Ovarian ca. OVCAR-5 | 0.6 | 0.5 | Small Intestine Pool | 0.4 | 0.4 |
| Ovarian ca. IGROV-1 | 0.0 | 0.0 | Stomach Pool | 0.1 | 0.3 |
| Ovarian ca. OVCAR-8 | 0.0 | 0.0 | Bone Marrow Pool | 0.2 | 0.2 |
| Ovary | 0.8 | 0.6 | Fetal Heart | 0.2 | 0.2 |
| Breast ca. MCF-7 | 0.0 | 0.0 | Heart Pool | 0.5 | 0.5 |
| Breast ca. MDA-MB-231 | 0.2 | 0.2 | Lymph Node Pool | 0.4 | 0.4 |
| Breast ca. BT 549 | 2.3 | 3.1 | Fetal Skeletal Muscle | 0.2 | 0.2 |
| Breast ca. T47D | 1.1 | 1.0 | Skeletal Muscle Pool | 1.7 | 1.9 |
| Breast ca. MDA-N | 0.2 | 0.2 | Spleen Pool | 2.3 | 2.6 |
| Breast Pool | 0.3 | 0.4 | Thymus Pool | 0.5 | 0.5 |
| Trachea | 0.9 | 1.0 | CNS cancer (glio/astro) U87-MG | 0.0 | 0.0 |
| Lung | 0.1 | 0.1 | CNS cancer (glio/astro) U-118- | 1.0 | 1.3 |
| | | | MG | | |
| Fetal Lung | 0.9 | 0.9 | CNS cancer (neuro; met) SK-N-AS | 1.2 | 1.5 |
| Lung ca. NCI-N417 | 0.0 | 0.0 | CNS cancer (astro) SF-539 | 0.3 | 0.4 |
| Lung ca. LX-1 | 0.0 | 0.0 | CNS cancer (astro) SNB-75 | 0.1 | 0.1 |
| Lung ca. NCI-H146 | 0.9 | 1.1 | CNS cancer (glio) SNB-19 | 0.0 | 0.0 |
| Lung ca. SHP-77 | 0.5 | 0.5 | CNS cancer (glio) SF-295 | 0.2 | 0.2 |
| Lung ca. A549 | 0.0 | 0.0 | Brain (Amygdala) Pool | 0.6 | 0.5 |
| Lung ca. NCI-H526 | 0.0 | 0.0 | Brain (cerebellum) | 0.1 | 0.2 |
| Lung ca. NCI-H23 | 0.0 | 0.0 | Brain (fetal) | 0.8 | 0.7 |
| Lung ca. NCI-H460 | 0.0 | 0.0 | Brain (Hippocampus) Pool | 0.5 | 0.5 |
| Lung ca. HOP-62 | 0.0 | 0.0 | Cerebral Cortex Pool | 0.7 | 0.7 |
| Lung ca. NCI-H522 | 0.0 | 0.0 | Brain (Substantia nigra) Pool | 0.8 | 0.7 |
| Liver | 0.1 | 0.1 | Brain (Thalamus) Pool | 0.8 | 0.9 |
| Fetal Liver | 4.7 | 4.1 | Brain (whole) | 1.3 | 1.3 |
| Liver ca. HepG2 | 0.0 | 0.0 | Spinal Cord Pool | 0.4 | 0.5 |
| Kidney Pool | 0.7 | 0.8 | Adrenal Gland | 0.7 | 0.8 |
| Fetal Kidney | 0.2 | 0.2 | Pituitary gland Pool | 0.3 | 0.3 |
| Renal ca. 786-0 | 0.0 | 0.0 | Salivary Gland | 0.5 | 0.4 |
| Renal ca. A498 | 0.2 | 0.2 | Thyroid (female) | 0.4 | 0.2 |
| Renal ca. ACHN | 0.0 | 0.0 | Pancreatic ca. CAPAN2 | 0.3 | 0.3 |
| Renal ca. UO-31 | 0.1 | 0.0 | Pancreas Pool | 0.4 | 0.3 |

TABLE BQF
Panel 1.3D
Column A - Rel. Exp.(%) Ag198O, Run 165534458

| Tissue Name | A | Tissue Name | A |
|---|---|---|---|
| Liver adenocarcinoma | 1.4 | Kidney (fetal) | 0.4 |
| Pancreas | 0.4 | Renal ca. 786-0 | 0.0 |
| Pancreatic ca. CAPAN 2 | 0.0 | Renal ca. A498 | 2.0 |
| Adrenal gland | 0.6 | Renal ca. RXF 393 | 0.7 |
| Thyroid | 0.7 | Renal ca. ACHN | 0.0 |
| Salivary gland | 1.0 | Renal ca. UO-31 | 0.0 |
| Pituitary gland | 0.5 | Renal ca. TK-10 | 0.1 |
| Brain (fetal) | 0.8 | Liver | 0.5 |
| Brain (whole) | 3.8 | Liver (fetal) | 5.3 |
| Brain (amygdala) | 2.9 | Liver ca. (hepatoblast) HepG2 | 0.0 |
| Brain (cerebellum) | 0.6 | Lung | 1.5 |
| Brain (hippocampus) | 3.2 | Lung (fetal) | 2.5 |
| Brain (substantia nigra) | 1.6 | Lung ca. (small cell) LX-1 | 0.0 |
| Brain (thalamus) | 5.1 | Lung ca. (small cell) NCI-H69 | 0.4 |
| Cerebral Cortex | 1.9 | Lung ca. (s.cell var.) SHP-77 | 0.5 |
| Spinal cord | 1.6 | Lung ca. (large cell)NCI-H460 | 0.0 |
| glio/astro U87-MG | 0.0 | Lung ca. (non-sm. cell) A549 | 0.0 |
| glio/astro U-118-MG | 1.8 | Lung ca. (non-s.cell) NCI-H23 | 0.0 |
| astrocytoma SW1783 | 0.0 | Lung ca. (non-s.cell) HOP-62 | 0.0 |
| neuro*; met SK-N-AS | 1.1 | Lung ca. (non-s.cl) NCI-H522 | 0.0 |
| astrocytoma SF-539 | 7.9 | Lung ca. (squam.) SW 900 | 1.5 |
| astrocytoma SNB-75 | 1.7 | Lung ca. (squam.) NCI-H596 | 0.1 |
| glioma SNB-19 | 0.0 | Mammary gland | 0.7 |
| glioma U251 | 0.7 | Breast ca.* (pl.ef) MCF-7 | 0.1 |
| glioma SF-295 | 0.0 | Breast ca.* (pl.ef) MDA-MB-231 | 0.3 |
| Heart (Fetal) | 0.1 | Breast ca.* (pl.ef) T47D | 0.0 |
| Heart | 1.2 | Breast ca. BT-549 | 1.1 |
| Skeletal muscle (Fetal) | 0.0 | Breast ca. MDA-N | 0.0 |
| Skeletal muscle | 1.0 | Ovary | 0.3 |
| Bone marrow | 4.5 | Ovarian ca. OVCAR-3 | 0.1 |
| Thymus | 1.2 | Ovarian ca. OVCAR-4 | 0.1 |
| Spleen | 1.4 | Ovarian ca. OVCAR-5 | 0.3 |
| Lymph node | 2.9 | Ovarian ca. OVCAR-8 | 0.0 |
| Colorectal | 0.3 | Ovarian ca. IGROV-1 | 0.0 |
| Stomach | 1.4 | Ovarian ca. (ascites) SK-OV-3 | 0.4 |
| Small intestine | 1.2 | Uterus | 1.8 |
| Colon ca. SW480 | 0.0 | Placenta | 1.3 |
| Colon ca.* SW620 (SW480 met) | 0.0 | Prostate | 0.3 |
| Colon ca. HT29 | 0.0 | Prostate ca.* (bone met) PC-3 | 0.1 |
| Colon ca. HCT-116 | 0.1 | Testis | 1.2 |
| Colon Ca. CaCo-2 | 0.0 | Melanoma Hs688(A).T | 0.1 |
| CC Well to Mod Diff (ODO3866) | 0.6 | Melanoma* (met) Hs688(B).T | 0.1 |
| Colon Ca. HCC-2998 | 12.0 | Melanoma UACC-62 | 1.0 |
| Gastric ca. (liver met) NCI-N87 | 100.0 | Melanoma M14 | 0.3 |
| Bladder | 8.2 | Melanoma LOX IMVI | 0.1 |
| Trachea | 2.0 | Melanoma* (met) SK-MEL-5 | 0.1 |
| Kidney | 0.4 | Adipose | 0.6 |

TABLE BQG
Panel 2D
Column A - Rel. Exp.(%) Ag1980, Run 169484147

| Tissue Name | A | Tissue Name | A |
|---|---|---|---|
| Normal Colon | 14.7 | Kidney Margin 8120608 | 1.1 |
| CC Well to Mod Duff (ODO3866) | 3.2 | Kidney Cancer 8120613 | 1.4 |
| CC Margin (ODO3866) | 6.3 | Kidney Margin 8120614 | 1.5 |
| CC Gr.2 rectosigmoid (ODO3868) | 3.5 | Kidney Cancer 9010320 | 6.2 |
| CC Margin (ODO3868) | 3.3 | Kidney Margin 9010321 | 2.6 |
| CC Mod Diff (ODO3920) | 1.3 | Normal Uterus | 1.2 |
| CC Margin (ODO3920) | 1.5 | Uterine Cancer 064011 | 3.4 |
| CC Gr.2 ascend colon (ODO3921) | 3.7 | Normal Thyroid | 4.7 |
| CC Margin (ODO3921) | 1.8 | Thyroid Cancer | 12.1 |
| CC from Partial Hepatectomy | 5.0 | Thyroid Cancer A302152 | 1.6 |
| (ODO4309) Mets | | | |
| Liver Margin (ODO4309) | 16.7 | Thyroid Margin A302153 | 4.4 |
| Colon mets to lung (OD04451-01) | 15.0 | Normal Breast | 4.5 |
| Lung Margin (OD04451-02) | 18.4 | Breast Cancer | 8.1 |
| Normal Prostate 6546-1 | 5.2 | Breast Cancer (OD04590-01) | 11.8 |
| Prostate Cancer (OD04410) | 9.1 | Breast Cancer Mets (OD04590-03) | 7.9 |
| Prostate Margin (OD04410) | 3.1 | Breast Cancer Metastasis | 5.3 |
| Prostate Cancer (OD04720-01) | 3.2 | Breast Cancer | 18.0 |
| Prostate Margin (OD04720-02) | 6.4 | Breast Cancer | 2.7 |
| Normal Lung | 14.8 | Breast Cancer 9100266 | 6.2 |
| Lung Met to Muscle (ODO4286) | 3.4 | Breast Margin 9100265 | 1.9 |
| Muscle Margin (ODO4286) | 2.8 | Breast Cancer A209073 | 6.5 |
| Lung Malignant Cancer (OD03126) | 5.2 | Breast Margin A209073 | 1.5 |
| Lung Margin (OD03126) | 21.5 | Normal Liver | 2.3 |
| Lung Cancer (OD04404) | 53.6 | Liver Cancer | 6.2 |
| Lung Margin (OD04404) | 6.8 | Liver Cancer 1025 | 6.5 |
| Lung Cancer (OD04565) | 6.8 | Liver Cancer 1026 | 2.1 |
| Lung Margin (OD04565) | 3.6 | Liver Cancer 6004-T | 6.0 |
| Lung Cancer (OD04237-01) | 10.1 | Liver Tissue 6004-N | 2.9 |
| Lung Margin (OD04237-02) | 24.5 | Liver Cancer 6005-T | 3.3 |
| Ocular Mel Met to Liver (ODO4310) | 0.2 | Liver Tissue 6005-N | 1.7 |
| Liver Margin (ODO4310) | 3.5 | Normal Bladder | 100.0 |
| Melanoma Metastasis | 1.8 | Bladder Cancer | 0.6 |
| Lung Margin (OD04321) | 10.3 | Bladder Cancer | 6.3 |
| Normal Kidney | 11.4 | Bladder Cancer (OD04718-01) | 74.2 |
| Kidney Ca, Nuclear grade 2 (OD04338) | 5.3 | Bladder Normal Adjacent | 5.1 |
| | | (OD04718-03) | |
| Kidney Margin (OD04338) | 4.2 | Normal Ovary | 3.5 |
| Kidney Ca Nuclear grade ½ (OD04339) | 1.5 | Ovarian Cancer | 11.7 |
| Kidney Margin (OD04339) | 4.6 | Ovarian Cancer (OD04768-07) | 99.3 |
| Kidney Ca, Clear cell type (OD04340) | 11.1 | Ovary Margin (OD04768-08) | 0.9 |
| Kidney Margin (OD04340) | 3.5 | Normal Stomach | 10.7 |
| Kidney Ca, Nuclear grade 3 (OD04348) | 59.9 | Gastric Cancer 9060358 | 3.0 |
| Kidney Margin (OD04348) | 81.2 | Stomach Margin 9060359 | 9.9 |
| Kidney Cancer (OD04622-01) | 6.1 | Gastric Cancer 9060395 | 6.9 |
| Kidney Margin (OD04622-03) | 1.1 | Stomach Margin 9060394 | 7.7 |
| Kidney Cancer (OD04450-01) | 1.5 | Gastric Cancer 9060397 | 7.2 |
| Kidney Margin (OD04450-03) | 1.7 | Stomach Margin 9060396 | 10.4 |
| Kidney Cancer 8120607 | 0.9 | Gastric Cancer 064005 | 19.9 |

TABLE BQH
Panel 4.1D
Column A - Rel. Exp.(%) Ag3909, Run 170127176

| Tissue Name | A | Tissue Name | A |
|---|---|---|---|
| Secondary Th1 act | 0.9 | HUVEC IL-1beta | 0.1 |
| Secondary Th2 act | 32.3 | HUVEC IFN gamma | 0.9 |
| Secondary Tr1 act | 3.6 | HUVEC TNF alpha + IFN gamma | 10.5 |
| Secondary Th1 rest | 5.5 | HUVEC TNF alpha + IL4 | 0.4 |
| Secondary Th2 rest | 2.0 | HUVEC IL-11 | 0.0 |
| Secondary Tr1 rest | 4.8 | Lung Microvascular EC none | 0.2 |
| Primary Th1 act | 1.3 | Lung Microvascular EC TNFalpha + IL- | 0.6 |
| | | 1beta | |
| Primary Th2 act | 2.0 | Microvascular Dermal EC none | 0.1 |
| Primary Tr1 act | 1.1 | Microsvasular Dermal EC TNFalpha + IL- | 0.4 |
| | | 1beta | |
| Primary Th1 rest | 3.5 | Bronchial epithelium TNFalpha + IL1beta | 0.0 |
| Primary Th2 rest | 0.3 | Small airway epithelium none | 0.0 |
| Primary Tr1 rest | 2.5 | Small airway epithelium TNFalpha + IL- | 0.0 |
| | | 1beta | |
| CD45RA CD4 lymphocyte act | 9.3 | Coronery artery SMC rest | 0.0 |
| CD45RO CD4 lymphocyte act | 12.4 | Coronery artery SMC TNFalpha + IL-1beta | 0.0 |
| CD8 lymphocyte act | 1.2 | Astrocytes rest | 0.0 |
| Secondary CD8 lymphocyte rest | 4.7 | Astrocytes TNFalpha + IL-1beta | 0.5 |
| Secondary CD8 lymphocyte act | 0.7 | KU-812 (Basophil) rest | 1.4 |
| CD4 lymphocyte none | 0.5 | KU-812 (Basophil) PMA/ionomycin | 4.7 |
| 2ry Th1/Th2/Tr1 anti-CD95 | 1.2 | CCD1106 (Keratinocytes) none | 0.4 |
| CH11 | | | |
| LAK cells rest | 2.8 | CCD1106 (Keratinocytes) TNFalpha + IL- | 7.7 |
| | | 1beta | |
| LAK cells IL-2 | 7.5 | Liver cirrhosis | 0.1 |
| LAK cells IL-2 + IL-12 | 7.1 | NCI-H292 none | 0.1 |
| LAK cells IL-2 + IFN gamma | 9.5 | NCI-H292 IL-4 | 0.3 |
| LAK cells IL-2 + IL-18 | 11.7 | NCI-H292 IL-9 | 0.4 |
| LAK cells PMA/ionomycin | 6.9 | NCI-H292 IL-13 | 0.5 |
| NK Cells IL-2 rest | 11.3 | NCI-H292 IFN gamma | 2.0 |
| Two Way MLR 3 day | 14.0 | HPAEC none | 0.0 |
| Two Way MLR 5 day | 5.3 | HPAEC TNF alpha + IL-1 beta | 2.9 |
| Two Way MLR 7 day | 1.7 | Lung fibroblast none | 0.0 |
| PBMC rest | 0.4 | Lung fibroblast TNF alpha + IL-1 beta | 9.5 |
| PBMC PWM | 2.7 | Lung fibroblast IL-4 | 0.0 |
| PBMC PHA-L | 1.1 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 3.6 |
| B lymphocytes PWM | 0.9 | Dermal fibroblast CCD1070 rest | 0.0 |
| B lymphocytes CD40L and IL-4 | 0.5 | Dermal fibroblast CCD1070 TNF alpha | 1.1 |
| EOL-1 dbcAMP | 0.6 | Dermal fibroblast CCD1070 IL-1 beta | 0.8 |
| EOL-1 dbcAMP | 0.0 | Dermal fibroblast IFN gamma | 6.3 |
| PMA/ionomycin | | | |
| Dendritic cells none | 0.6 | Dermal fibroblast IL-4 | 0.3 |
| Dendritic cells LPS | 20.0 | Dermal Fibroblasts rest | 0.1 |
| Dendritic cells anti-CD40 | 0.3 | Neutrophils TNFa + LPS | 0.1 |
| Monocytes rest | 2.7 | Neutrophils rest | 0.3 |
| Monocytes LPS | 100.0 | Colon | 0.5 |
| Macrophages rest | 0.9 | Lung | 0.9 |
| Macrophages LPS | 36.9 | Thymus | 1.0 |
| HUVEC none | 0.0 | Kidney | 0.4 |
| HUVEC starved | 0.1 | | |

TABLE BQJ
Panel 5 Islet
Column A - Rel. Exp.(%) Ag3909, Run 242413199

| Tissue Name | A | Tissue Name | A |
|---|---|---|---|
| 97457 Patient-02go adipose | 90.1 | 94709 Donor 2 AM - A adipose | 0.0 |
| 97476 Patient-07sk skeletal muscle | 22.2 | 94710 Donor 2 AM - B adipose | 1.8 |
| 97477 Patient-07ut uterus | 18.2 | 94711 Donor 2 AM - C adipose | 0.0 |
| 97478 Patient-07pl placenta | 53.6 | 94712 Donor 2 AD - A adipose | 1.6 |
| 99167 Bayer Patient 1 | 100.0 | 94713 Donor 2 AD - B adipose | 1.2 |
| 97482 Patient-08ut uterus | 44.8 | 94714 Donor 2 AD - C adipose | 0.0 |
| 97483 Patient-08pl placenta | 74.2 | 94742 Donor 3 U - A Mesenchymal | 3.1 |
| | | Stem Cells | |
| 97486 Patient-09sk skeletal muscle | 9.3 | 94743 Donor 3 U - B Mesenchymal | 3.7 |
| | | Stem Cells | |
| 97487 Patient-09ut uterus | 24.1 | 94730 Donor 3 AM - A adipose | 1.7 |
| 97488 Patient-09pl placenta | 44.1 | 94731 Donor 3 AM - B adipose | 3.6 |
| 97492 Patient-10ut uterus | 47.0 | 94732 Donor 3 AM - C adipose | 0.0 |
| 97493 Patient-10pl placenta | 68.3 | 94733 Donor 3 AD - A adipose | 0.0 |
| 97495 Patient-11go adipose | 17.4 | 94734 Donor 3 AD - B adipose | 0.0 |
| 97496 Patient-11sk skeletal muscle | 22.8 | 94735 Donor 3 AD - C adipose | 0.0 |
| 97497 Patient-11ut uterus | 24.3 | 77138 Liver HepG2untreated | 1.8 |
| 97498 Patient-11pl placenta | 15.4 | 73556 Heart Cardiac stromal cells | 0.0 |
| | | (primary) | |
| 97500 Patient-12go adipose | 62.4 | 81735 Small Intestine | 97.3 |
| 97501 Patient-12sk skeletal muscle | 79.6 | 72409 Kidney Proximal Convoluted | 5.6 |
| | | Tubule | |
| 97502 Patient-12ut uterus | 30.6 | 82685 Small intestine Duodenum | 54.3 |
| 97503 Patient-12pl placenta | 29.7 | 90650 Adrenal Adrenocortical | 10.0 |
| | | adenoma | |
| 94721 Donor 2 U - A Mesenchymal | 0.0 | 72410 Kidney HRCE | 8.3 |
| Stem Cells | | | |
| 94722 Donor 2 U - B Mesenchymal | 0.0 | 72411 Kidney HRE | 28.3 |
| Stem Cells | | | |
| 94723 Donor 2 U - C Mesenchymal | 0.0 | 73139 Uterus Uterine smooth muscle | 0.0 |
| Stem Cells | | cells | |

AI.05 chondrosarcoma Summary: Ag1980 Highest expression of this gene was detected in the IL-1 beta/TNF-a treated chondrosarcoma cell line (SW1353). Expression of this gene was up-regulated upon IL-1 treatment, a potent activator of pro-inflammatory cytokines and matrix metalloproteinases, which participate in the destruction of cartilage observed in Osteoarthritis (OA). Modulation of the expression of this transcript in chondrocytes by either small molecules, antibody, or protein therapeutics is useful for preventing the degeneration of cartilage observed in OA.

AI_comprehensive panel_v1.0 Summary: Ag1980 Highest expression was detected in normal tissue adjacent to psoriasis (CTs=30.5-31.2). Expression of this gene was induced in bone tissue, synovial fluid, synovial fluid cells and synovium from arthritis patients (rheumatoid-RA and osteoarthritis-OA), while the expression of this transcript in these samples from normal patients was much lower. Other tissues including skin and lung also expressed this transcript. However, a consistent expression in diseased tissue, as compared to adjacent tissue or normal lung, is not apparent. This may be due to contamination with activated monocytes which highly express this transcript (see panel 4.1D) Modulation of the expression of this transcript in chondrocytes by either small molecules, antibody, or protein therapeutics is useful for treating rheumatoid arthritis and preventing the degeneration of cartilage observed in OA.

General_screening_panel_v1.4 Summary: Ag3909 Highest expression of the CG94235-01 gene was detected in a gastric cancer cell line (CTs=23.6-24.4). Thus, expression of this gene is useful as a marker of gastric cancer. This gene encodes a putative thymidylate kinase, a DNA synthesis enzyme necessary for cell growth. Therapeutic modulation of this gene, expressed protein and/or use of antibodies or small molecule drugs targeting the gene or gene product are useful in the treatment of gastric cancer.

Among tissues with metabolic function, this gene was expressed at moderate to low levels in pituitary, adipose, adrenal gland, pancreas, thyroid, and adult and fetal skeletal muscle, heart, and liver. The widespread expression among these tissues indicates that this gene product may play a role in normal neuroendocrine and metabolic disorders. Therapeutic modulation of this gene, expressed protein and/or use of antibodies or small molecule drugs targeting the gene or gene product are useful in the treatment of neuroendocrine disorders or metabolic diseases, such as obesity and diabetes.

In addition, this gene was expressed at much higher levels in fetal lung, liver and skeletal muscle tissue (CTs=28-30) when compared to expression in the adult counterpart (CTs=32.5-35). Thus, expression of this gene is useful for distinguishing between the fetal and adult source of these tissues.

This gene was also expressed at moderate to low levels in the CNS, including the hippocampus, thalamus, substantia nigra, amygdala, cerebellum and cerebral cortex. Therapeutic modulation of this gene, expressed protein and/or use of antibodies or small molecule drugs targeting the gene or gene product are useful in the treatment of neurologic disorders, such as Alzheimer's disease, Parkinson's disease, schizophrenia, multiple sclerosis, stroke and epilepsy.

Panel 1.3D Summary: Ag1980 Highest expression of the CG94235-01 gene in this panel was seen in a gastric cancer cell line (CT=26). Overall, expression was in reasonable agreement with the results in Panel 1.4. Moderate to low levels of expression were seen in metabolic tissues including adipose, adult and fetal liver, skeletal muscle, heart, pituitary, thyroid, adrenal and pituitary. Moderate to low levels of expression were seen in all CNS regions examined.

In addition, higher levels of expression were seen in fetal liver (CT=30.2) when compared to expression in adult liver (CT=33.7). Thus, expression of this gene is useful for distinguishing between the adult and fetal sources of this tissue.

Panel 2D Summary: Ag1980 Highest expression of the CG94235-01 gene was seen in normal bladder (CT=27.3). In addition, higher levels of expression were seen in ovarian, bladder and lung cancers when compared to expression in normal adjacent tissue. Thus, expression of this gene is useful as a marker of these cancers. Therapeutic modulation of this gene, expressed protein and/or use of antibodies or small molecule drugs targeting the gene or gene product are useful in the treatment of ovarian, bladder and lung cancers.

Panel 4.1D Summary: Ag3909 Highest expression of the CG94235-01 gene was seen in LPS treated monocytes (CT=25.4). Prominent levels of expression were also seen in LPS activated macrophages and dendritic cells. This transcript encodes a protein that may be important in the normal regulation of cytokines. Inappropriate regulation of the protein encoded by this gene may result in the enhanced and uncontrolled expression of inflammatory cytokines. Therapeutic modulation of this gene, expressed protein and/or use of antibodies or small molecule drugs targeting the gene or gene product are useful in the treatment of osteoarthritis and rheumatoid arthritis.

Panel 5 Islet Summary: Ag3909 Highest expression of the CG94235-01 gene was seen in islet cells (CT=33.4). Low but significant levels of expression were seen in other metabolic tissues, including adipose, placenta and skeletal muscle. Please see Panel 1.4 for discussion of this gene in metabolic disease.

BR. CG95175-01: Ephrin Type-A Receptor 7 Precursor.

Expression of gene CG95175-01 was assessed using the primer-probe sets Ag3992 and Ag612, described in Tables BRA and BRB. Results of the RTQ-PCR runs are shown in Tables BRC, BRD, BRE and BRF.

TABLE BRA
Probe Name Ag3992

| | | | | SEQ |
|---|---|---|---|---|
| | | | Start | ID |
| Primers | Sequences | Length | Position | No |
| Forward | 5′-accactatggtgaggctacaga-3′ | 22 | 2427 | 1467 |
| Probe | TET-5′-ctatgggccgctcccgagacact-3′- | 23 | 2466 | 1468 |
| | TAMRA | | | |
| Reverse | 5′-agagctgaagtggccaaact-3′ | 20 | 2491 | 1469 |

TABLE BRB
Probe Name Ag612

| | | | | SEQ |
|---|---|---|---|---|
| | | | Start | ID |
| Primers | Sequences | Length | Position | No |
| Forward | 5′-gccgctcccgagacactt-3′ | 18 | 2472 | 1470 |
| Probe | TET-5′-ccacttcagctctgccagtgacgtg-3′ | 25 | 2498 | 1471 |
| | TAMRA | | | |
| Reverse | 5′-cccacatgatgatgccgaa-3′ | 19 | 2529 | 1472 |

TABLE BRC
CNS_neurodegeneration_v1.0
Column A - Rel. Exp.(%) Ag612, Run 309606071

| Tissue Name | A |
|---|---|
| AD 1 Hippo | 46.0 |
| AD 2 Hippo | 37.1 |
| AD 3 Hippo | 9.5 |
| AD 4 Hippo | 33.9 |
| AD 5 Hippo | 59.5 |
| AD 6 Hippo | 41.8 |
| Control 2 Hippo | 40.9 |
| Control 4 Hippo | 21.6 |
| Control (Path) 3 Hippo | 15.9 |
| AD 1 Temporal Ctx | 26.1 |
| AD 2 Temporal Ctx | 28.9 |
| AD 3 Temporal Ctx | 12.3 |
| AD 4 Temporal Ctx | 62.9 |
| AD 5 Inf Temporal Ctx | 57.4 |
| AD 5 Sup Temporal Ctx | 33.7 |
| AD 6 Inf Temporal Ctx | 50.7 |
| AD 6 Sup Temporal Ctx | 68.8 |
| Control 1 Temporal Ctx | 9.7 |
| Control 2 Temporal Ctx | 54.7 |
| Control 3 Temporal Ctx | 40.3 |
| Control 3 Temporal Ctx | 43.2 |
| AH3 3975 | 97.3 |
| AH3 3954 | 86.5 |
| AH3 4624 | 31.0 |
| AH3 4640 | 100.0 |
| AD 1 Occipital Ctx | 11.0 |
| AD 2 Occipital Ctx (Missing) | 6.8 |
| AD 3 Occipital Ctx | 8.3 |
| AD 4 Occipital Ctx | 35.6 |
| AD 5 Occipital Ctx | 43.5 |
| AD 5 Occipital Ctx | 21.5 |
| Control 1 Occipital Ctx | 8.2 |
| Control 2 Occipital Ctx | 36.6 |
| Control 3 Occipital Ctx | 29.5 |
| Control 4 Occipital Ctx | 20.3 |
| Control (Path) 1 Occipital Ctx | 81.2 |
| Control (Path) 2 Occipital Ctx | 37.4 |
| Control (Path) 3 Occipital Ctx | 17.8 |
| Control (Path) 4 Occipital Ctx | 29.9 |
| Control 1 Parietal Ctx | 37.6 |
| Control 2 Parietal Ctx | 41.5 |
| Control 3 Parietal Ctx | 54.0 |
| Control (Path) 1 Parietal Ctx | 99.3 |
| Control (Path) 2 Parietal Ctx | 52.9 |
| Control (Path) 3 Parietal Ctx | 11.6 |
| Control (Path) 4 Parietal Ctx | 56.6 |

TABLE BRE
Panel 1.3D
Column A - Rel. Exp.(%) Ag612, Run 165720641

| Tissue Name | A | Tissue Name | A |
|---|---|---|---|
| Liver adenocarcinoma | 8.0 | Kidney (fetal) | 0.0 |
| Pancreas | 2.8 | Renal ca. 786-0 | 0.0 |
| Pancreatic ca. CAPAN 2 | 0.7 | Renal ca. A498 | 0.0 |
| Adrenal gland | 0.0 | Renal ca. RXF 393 | 1.7 |
| Thyroid | 0.0 | Renal ca. ACHN | 5.9 |
| Salivary gland | 4.0 | Renal ca. UO-31 | 0.0 |
| Pituitary gland | 16.0 | Renal ca. TK-10 | 6.7 |
| Brain (fetal) | 67.8 | Liver | 0.0 |
| Brain (whole) | 17.9 | Liver (fetal) | 0.0 |
| Brain (amygdala) | 32.5 | Liver ca. (hepatoblast) HepG2 | 0.0 |
| Brain (cerebellum) | 7.6 | Lung | 0.0 |
| Brain (hippocampus) | 41.2 | Lung (fetal) | 0.0 |
| Brain (substantia nigra) | 10.5 | Lung ca. (small cell) LX-1 | 1.6 |
| Brain (thalamus) | 22.1 | Lung ca. (small cell) NCI-H69 | 0.6 |
| Cerebral Cortex | 27.7 | Lung ca. (s. cell var.) SHP-77 | 4.6 |
| Spinal cord | 5.9 | Lung ca. (large cell)NCI-H460 | 2.4 |
| glio/astro U87-MG | 0.0 | Lung ca. (non-sm. cell) A549 | 0.0 |
| glio/astro U-118-MG | 0.0 | Lung ca. (non-s. cell) NCI-H23 | 5.2 |
| astrocytoma SW1783 | 0.0 | Lung ca. (non-s. cell) HOP-62 | 0.0 |
| neuro*; met SK-N-AS | 0.6 | Lung ca. (non-s. cl) NCI-H522 | 10.7 |
| astrocytoma SF-539 | 0.0 | Lung ca. (squam.) SW 900 | 6.9 |
| astrocytoma SNB-75 | 13.7 | Lung ca. (squam.) NCI-H596 | 0.0 |
| glioma SNB-19 | 0.0 | Mammary gland | 4.2 |
| glioma U251 | 0.0 | Breast ca.* (pl. ef) MCF-7 | 7.9 |
| glioma SF-295 | 0.0 | Breast ca.* (pl. ef) MDA-MB-231 | 0.6 |
| Heart (Fetal) | 0.0 | Breast ca.* (pl. ef) T47D | 1.9 |
| Heart | 0.7 | Breast ca. BT-549 | 0.0 |
| Skeletal muscle (Fetal) | 0.0 | Breast ca. MDA-N | 0.0 |
| Skeletal muscle | 0.0 | Ovary | 0.7 |
| Bone marrow | 0.0 | Ovarian ca. OVCAR-3 | 7.1 |
| Thymus | 0.1 | Ovarian ca. OVCAR-4 | 0.1 |
| Spleen | 1.3 | Ovarian ca. OVCAR-5 | 2.7 |
| Lymph node | 0.9 | Ovarian ca. OVCAR-8 | 3.5 |
| Colorectal | 24.3 | Ovarian ca. IGROV-1 | 0.0 |
| Stomach | 9.5 | Ovarian ca. (ascites) SK-OV-3 | 12.4 |
| Small intestine | 11.3 | Uterus | 0.0 |
| Colon ca. SW480 | 3.7 | Placenta | 0.0 |
| Colon ca.* SW620 (SW480 met) | 1.8 | Prostate | 4.2 |
| Colon ca. HT29 | 0.0 | Prostate ca.* (bone met) PC-3 | 2.9 |
| Colon ca. HCT-116 | 0.8 | Testis | 100.0 |
| Colon Ca. CaCo-2 | 1.7 | Melanoma Hs688(A).T | 0.0 |
| CC Well to Mod Diff (ODO3866) | 8.7 | Melanoma* (met) Hs688(B).T | 0.0 |
| Colon ca. HCC-2998 | 9.6 | Melanoma UACC-62 | 0.0 |
| Gastric ca. (liver met) NCI-N87 | 15.1 | Melanoma M14 | 0.0 |
| Bladder | 3.3 | Melanoma LOX IMVI | 0.0 |
| Trachea | 3.8 | Melanoma* (met) SK-MEL-5 | 0.7 |
| Kidney | 0.0 | Adipose | 0.6 |

TABLE BRF
Panel 4D
Column A - Rel. Exp.(%) Ag612, Run 145645058

| Tissue Name | A | Tissue Name | A |
|---|---|---|---|
| Secondary Th1 act | 0.0 | HUVEC IL-1beta | 0.0 |
| Secondary Th2 act | 12.2 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + IL- | 0.0 |
| | | 1beta | |
| Primary Th2 act | 0.0 | Microvascular Dermal EC none | 0.0 |
| Primary Tr1 act | 0.0 | Microsvasular Dermal EC TNFalpha + IL- | 0.0 |
| | | 1beta | |
| Primary Th1 rest | 0.0 | Bronchial epithelium TNFalpha + IL1beta | 0.0 |
| Primary Th2 rest | 0.0 | Small airway epithelium none | 0.0 |
| Primary Tn rest | 0.0 | Small airway epithelium TNFalpha + IL- | 4.6 |
| | | 1beta | |
| CD45RA CD4 lymphocyte act | 0.0 | Coronery artery SMC rest | 0.0 |
| CD45RO CD4 lymphocyte act | 4.6 | Coronery artery SMC TNFalpha + IL-1beta | 0.0 |
| CD8 lymphocyte act | 5.3 | Astrocytes rest | 0.0 |
| Secondary CD8 lymphocyte rest | 5.3 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 | 0.0 | CCD1106 (Keratinocytes) none | 0.0 |
| CH11 | | | |
| LAK cells rest | 9.7 | CCD1106 (Keratinocytes) TNFalpha + IL- | 1.0 |
| | | 1beta | |
| LAK cells IL-2 | 0.0 | Liver cirrhosis | 61.6 |
| LAK cells IL-2 + IL-12 | 0.0 | Lupus kidney | 0.0 |
| LAK cells IL-2 + IFN gamma | 5.1 | NCI-H292 none | 32.5 |
| LAK cells IL-2 + IL-18 | 0.0 | NCI-H292 IL-4 | 46.7 |
| LAK cells PMA/ionomycin | 0.0 | NCI-H292 IL-9 | 58.6 |
| NK Cells IL-2 rest | 0.0 | NCI-H292 IL-13 | 89.5 |
| Two Way MLR 3 day | 0.0 | NCI-H292 IFN gamma | 100.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 beta | 0.0 |
| PBMC PHA-L | 8.9 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 9.0 |
| B lymphocytes PWM | 8.5 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF alpha | 0.0 |
| EOL-1 dbcAMP | 0.0 | Dermal fibroblast CCD1070 IL-1 beta | 0.0 |
| PMA/ionomycin | | | |
| Dendritic cells none | 5.3 | Dermal fibroblast IFN gamma | 0.0 |
| Dendritic cells LPS | 4.5 | Dermal fibroblast IL-4 | 0.0 |
| Dendritic cells anti-CD40 | 0.0 | IBD Colitis 2 | 10.7 |
| Monocytes rest | 0.0 | IBD Crohn's | 0.0 |
| Monocytes LPS | 1.2 | Colon | 51.8 |
| Macrophages rest | 0.0 | Lung | 24.7 |
| Macrophages LPS | 0.0 | Thymus | 4.1 |
| HUVEC none | 0.0 | Kidney | 4.6 |
| HUVEC starved | 0.0 | | |

CNS_neurodegeneration_v1.0 Summary: Ag612 This gene was found to be down-regulated in the temporal cortex of Alzheimer's disease patients. Therapeutic modulation of this gene, expressed protein and/or use of antibodies or small molecule drugs targeting the gene or gene product are useful in the treatment of dementia/memory loss associated with this disease and neuronal death.

Panel 1.3D Summary: Ag612 Highest expression of the CG95175-01 gene was detected in testis (CT=29). In addition, high expression of this gene was also detected in all the region of the central nervous system examined, and in a cluster of lung cancer, colon cancer, renal cancer, a liver cancer, breast cancer, ovarian cancer and an astrocytoma cell lines, pancreas, pituitary gland, and the gastrointestinal tract. Therapeutic modulation of this gene, expressed protein and/or use of antibodies or small molecule drugs targeting the gene or gene product are useful in the treatment of diseases of the central nervous system including Alzheimer's disease.

Panel 4D Summary: Ag612 Highest expression of this gene was detected in IFN gama treated NCI-H292 cells (CT=33). Moderate to low expression of this gene was also seen in cytokine treated and untreated NCI-H292 cells, liver cirrhosis and colon tissue samples. Therapeutic modulation of this gene, expressed protein and/or use of antibodies or small molecule drugs targeting the gene or gene product are useful in the treatment of chronic obstructive pulmonary disease, asthma, allergy, and emphysema, liver cirrhosis, autoimmune and inflammatory disease affecting colon including Crohn's disease and ulcerative colitis.

BS. CG9963801: Sodium/Nucleoside Cotransporter 1.

Expression of gene CG99638-01 was assessed using the primer-probe set Ag1521, described in Table BSA. Results of the RTQ-PCR runs are shown in Tables BSB, BSC and BSD.

TABLE BSA
Probe Name Ag1521

| | | | | SEQ |
|---|---|---|---|---|
| | | | Start | ID |
| Primers | Sequences | Length | Position | No |
| Forward | 5′-tggttttcttcagcactgtgat-3′ | 22 | 889 | 1473 |
| Probe | TET-5′-catgctgtactaccctggactgatgca-3′- | 27 | 914 | 1474 |
| | TAMRA | | | |
| Reverse | 5′-catgatccatccaacctttcta-3′ | 22 | 950 | 1475 |

TABLE BSB
Panel 1.3D
Column A - Rel. Exp.(%) Ag1521, Run 165544920

| Tissue Name | A | Tissue Name | A |
|---|---|---|---|
| Liver adenocarcinoma | 0.0 | Kidney (fetal) | 2.6 |
| Pancreas | 29.3 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 1.6 | Renal ca. UO-31 | 0.9 |
| Pituitary gland | 1.9 | Renal ca. TK-10 | 0.0 |
| Brain (fetal) | 0.0 | Liver | 0.0 |
| Brain (whole) | 1.2 | Liver (fetal) | 1.6 |
| Brain (amygdala) | 0.0 | Liver ca. (hepatoblast) HepG2 | 0.0 |
| Brain (cerebellum) | 0.0 | Lung | 0.6 |
| Brain (hippocampus) | 3.1 | Lung (fetal) | 3.3 |
| Brain (substantia nigra) | 0.0 | Lung ca. (small cell) LX-1 | 0.0 |
| Brain (thalamus) | 1.8 | Lung ca. (small cell) NCI-H69 | 0.0 |
| Cerebral Cortex | 0.0 | Lung ca. (s. cell var.) SHP-77 | 0.0 |
| Spinal cord | 0.7 | Lung ca. (large cell)NCI-H460 | 0.0 |
| glio/astro U87-MG | 0.0 | Lung ca. (non-sm. cell) A549 | 0.0 |
| glio/astro U-118-MG | 4.2 | Lung ca. (non-s. cell) NCI-H23 | 0.0 |
| astrocytoma SW1783 | 4.9 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.3 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 7.8 |
| glioma U251 | 0.0 | Breast ca.* (pl. ef) MCF-7 | 0.6 |
| glioma SF-295 | 3.3 | Breast ca.* (pl. ef) MDA-MB-231 | 1.1 |
| Heart (Fetal) | 0.0 | Breast ca.* (pl. ef) T47D | 0.0 |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 100.0 | Ovarian ca. OVCAR-3 | 3.1 |
| Thymus | 0.7 | Ovarian ca. OVCAR-4 | 2.8 |
| Spleen | 1.4 | Ovarian ca. OVCAR-5 | 2.1 |
| Lymph node | 2.9 | Ovarian ca. OVCAR-8 | 0.0 |
| Colorectal | 5.3 | Ovarian ca. IGROV-l | 0.0 |
| Stomach | 5.3 | Ovarian ca. (ascites) SK-OV-3 | 0.6 |
| Small intestine | 4.6 | Uterus | 1.8 |
| Colon ca. SW480 | 0.0 | Placenta | 0.0 |
| Colon ca.* SW620 (SW480 met) | 0.0 | Prostate | 0.4 |
| Colon ca. HT29 | 5.4 | Prostate ca.* (bone met) PC-3 | 0.0 |
| Colon ca. HCT-116 | 0.0 | Testis | 2.0 |
| Colon ca. CaCo-2 | 0.5 | Melanoma Hs688(A).T | 0.0 |
| CC Well to Mod Diff (ODO3866) | 13.9 | Melanoma* (met) Hs688(B).T | 0.0 |
| Colon ca. HCC-2998 | 0.0 | Melanoma UACC-62 | 0.0 |
| Gastric ca. (liver met) NCI-N87 | 2.2 | Melanoma M14 | 0.0 |
| Bladder | 36.3 | Melanoma LOX IMVI | 0.0 |
| Trachea | 22.5 | Melanoma* (met) SK-MEL-5 | 0.0 |
| Kidney | 0.0 | Adipose | 14.5 |

TABLE BSC
Panel 2.2
Column A - Rel. Exp.(%) Ag1521, Run 173816642

| Tissue Name | A | Tissue Name | A |
|---|---|---|---|
| Normal Colon | 3.2 | Kidney Margin (OD04348) | 1.0 |
| Colon cancer (OD06064) | 9.9 | Kidney malignant cancer | 1.3 |
| | | (OD06204B) | |
| Colon Margin (OD06064) | 2.6 | Kidney normal adjacent tissue | 0.0 |
| | | (OD06204E) | |
| Colon cancer (OD06159) | 0.0 | Kidney Cancer (OD04450-01) | 0.0 |
| Colon Margin (OD06159) | 1.9 | Kidney Margin (OD04450-03) | 0.0 |
| Colon cancer (OD06297-04) | 1.1 | Kidney Cancer 8120613 | 0.0 |
| Colon Margin (OD06297-05) | 2.3 | Kidney Margin 8120614 | 0.0 |
| CC Gr.2 ascend colon (ODO3921) | 0.0 | Kidney Cancer 9010320 | 0.0 |
| CC Margin (ODO3921) | 1.7 | Kidney Margin 9010321 | 2.8 |
| Colon cancer metastasis (OD06104) | 9.5 | Kidney Cancer 8120607 | 0.0 |
| Lung Margin (OD06104) | 6.0 | Kidney Margin 8120608 | 0.0 |
| Colon mets to lung (OD04451-01) | 13.1 | Normal Uterus | 0.0 |
| Lung Margin (OD04451-02) | 3.0 | Uterine Cancer 064011 | 9.7 |
| Normal Prostate | 2.2 | Normal Thyroid | 0.0 |
| Prostate Cancer (OD04410) | 0.0 | Thyroid Cancer | 0.0 |
| Prostate Margin (OD04410) | 0.0 | Thyroid Cancer A302152 | 10.7 |
| Normal Ovary | 0.0 | Thyroid Margin A302153 | 0.0 |
| Ovarian cancer (OD06283-03) | 6.7 | Normal Breast | 100.0 |
| Ovarian Margin (OD06283-07) | 6.0 | Breast Cancer | 0.0 |
| Ovarian Cancer | 1.8 | Breast Cancer | 9.3 |
| Ovarian cancer (OD06145) | 6.9 | Breast Cancer (OD04590-01) | 1.3 |
| Ovarian Margin (OD06145) | 16.4 | Breast Cancer Mets (OD04590-03) | 0.0 |
| Ovarian cancer (OD06455-03) | 6.7 | Breast Cancer Metastasis | 1.4 |
| Ovarian Margin (0D06455-07) | 0.0 | Breast Cancer | 1.6 |
| Normal Lung | 0.0 | Breast Cancer 9100266 | 0.0 |
| Invasive poor diff. lung adeno | 13.9 | Breast Margin 9100265 | 6.9 |
| (ODO4945-01 | | | |
| Lung Margin (ODO4945-03) | 6.0 | Breast Cancer A209073 | 0.0 |
| Lung Malignant Cancer (OD03126) | 5.3 | Breast Margin A209073 | 22.4 |
| Lung Margin (OD03126) | 1.5 | Breast cancer (OD06083) | 20.6 |
| Lung Cancer (OD05014A) | 20.7 | Breast cancer node metastasis | 1.1 |
| | | (OD06083) | |
| Lung Margin (OD05014B) | 12.1 | Normal Liver | 1.5 |
| Lung cancer (OD06081) | 0.0 | Liver Cancer 1026 | 0.0 |
| Lung Margin (OD06081) | 1.4 | Liver Cancer 1025 | 0.0 |
| Lung Cancer (OD04237-01) | 1.8 | Liver Cancer 6004-T | 0.0 |
| Lung Margin (OD04237-02) | 3.6 | Liver Tissue 6004-N | 0.0 |
| Ocular Mel Met to Liver (ODO4310) | 0.0 | Liver Cancer 6005-T | 2.5 |
| Liver Margin (ODO4310) | 0.0 | Liver Tissue 6005-N | 3.1 |
| Melanoma Metastasis | 0.0 | Liver Cancer | 0.0 |
| Lung Margin (OD04321) | 0.0 | Normal Bladder | 15.2 |
| Normal Kidney | 0.0 | Bladder Cancer | 1.6 |
| Kidney Ca, Nuclear grade 2 | 0.0 | Bladder Cancer | 8.2 |
| (OD04338) | | | |
| Kidney Margin (OD04338) | 0.0 | Normal Stomach | 3.3 |
| Kidney Ca Nuclear grade 1/2 | 3.8 | Gastric Cancer 9060397 | 1.7 |
| (OD04339) | | | |
| Kidney Margin (OD04339) | 0.0 | Stomach Margin 9060396 | 0.0 |
| Kidney Ca, Clear cell type (OD04340) | 0.0 | Gastric Cancer 9060395 | 0.0 |
| Kidney Margin (OD04340) | 0.0 | Stomach Margin 9060394 | 8.2 |
| Kidney Ca, Nuclear grade 3 | 0.0 | Gastric Cancer 064005 | 0.0 |
| (OD04348) | | | |

TABLE BSD
Panel 4D
Column A - Rel. Exp.(%) Ag1521, Run 165725922

| Tissue Name | A | Tissue Name | A |
|---|---|---|---|
| Secondary Th1 act | 0.4 | HUVEC IL-1beta | 0.0 |
| Secondary Th2 act | 0.1 | HUVEC IFN gamma | 0.0 |
| Secondary Tr1 act | 0.1 | HUVEC TNF alpha + IFN gamma | 0.0 |
| Secondary Th1 rest | 0.2 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.4 | Lung Microvascular EC TNFalpha + IL- | 0.1 |
| | | 1beta | |
| Primary Th2 act | 0.1 | Microvascular Dermal EC none | 0.0 |
| Primary Tr1 act | 0.4 | Microsvasular Dermal EC TNFalpha + IL- | 0.0 |
| | | 1beta | |
| Primary Th1 rest | 2.5 | Bronchial epithelium TNFalpha + IL1beta | 2.6 |
| Primary Th2 rest | 0.6 | Small airway epithelium none | 1.1 |
| Primary Tr1 rest | 0.2 | Small airway epithelium TNFalpha + IL- | 15.1 |
| | | 1beta | |
| CD45RA CD4 lymphocyte act | 0.9 | Coronery artery SMC rest | 0.2 |
| CD45RO CD4 lymphocyte act | 0.6 | Coronery artery SMG TNFalpha + IL-1beta | 0.2 |
| CD8 lymphocyte act | 1.0 | Astrocytes rest | 0.6 |
| Secondary CD8 lymphocyte rest | 0.0 | Astrocytes TNFalpha + IL-1beta | 2.3 |
| Secondary CD8 lymphocyte act | 0.8 | KU-812 (Basophil) rest | 0.0 |
| CD4 lymphocyte none | 0.0 | KU-812 (Basophil) PMA/ionomycin | 0.1 |
| 2ry Th1/Th2/Tr1 anti-CD95 | 1.0 | CCD1106 (Keratinocytes) none | 0.2 |
| CH11 | | | |
| LAK cells rest | 5.4 | CCD1106 (Keratinocytes) TNFalpha + IL- | 16.3 |
| | | 1beta | |
| LAK cells IL-2 | 0.2 | Liver cirrhosis | 4.2 |
| LAK cells IL-2 + IL-12 | 1.3 | Lupus kidney | 0.2 |
| LAK cells IL-2 + IFN gamma | 3.6 | NCI-H292 none | 1.1 |
| LAK cells IL-2 + IL-18 | 0.7 | NCI-H292 IL-4 | 3.8 |
| LAK cells PMA/ionomycin | 5.5 | NCI-H292 IL-9 | 0.6 |
| NK Cells IL-2 rest | 0.0 | NCI-H292 IL-13 | 1.7 |
| Two Way MLR 3 day | 0.7 | NCI-H292 IFN gamma | 0.3 |
| Two Way MLR 5 day | 2.4 | HPAEC none | 0.0 |
| Two Way MIR 7 day | 2.4 | HPAEC TNF alpha + IL-1 beta | 0.0 |
| PBMC rest | 0.2 | Lung fibroblast none | 0.4 |
| PBMC PWM | 0.3 | Lung fibroblast TNF alpha + IL-1 beta | 0.1 |
| PBMC PHA-L | 0.1 | Lung fibroblast IL-4 | 0.3 |
| Ramos (B cell) none | 0.0 | Lung fibroblast IL-9 | 0.6 |
| Ramos (B cell) ionomycin | 0.0 | Lung fibroblast IL-13 | 0.2 |
| B lymphocytes PWM | 0.5 | Lung fibroblast IFN gamma | 0.5 |
| B lymphocytes CD40L and IL-4 | 0.1 | Dermal fibroblast CCD1070 rest | 0.7 |
| EOL-1 dbcAMP | 0.0 | Dermal fibroblast CCD1070 TNF alpha | 4.4 |
| EOL-1 dbcAMP | 0.0 | Dermal fibroblast CCD1070 IL-1 beta | 0.4 |
| PMA/ionomycin | | | |
| Dendritic cells none | 35.6 | Dermal fibroblast IFN gamma | 0.5 |
| Dendritic cells LPS | 8.4 | Dermal fibroblast IL-4 | 0.4 |
| Dendritic cells anti-CD40 | 19.3 | IBD Colitis 2 | 0.0 |
| Monocytes rest | 0.8 | IBD Crohn's | 0.7 |
| Monocytes LPS | 0.2 | Colon | 5.8 |
| Macrophages rest | 100.0 | Lung | 0.1 |
| Macrophages LPS | 20.9 | Thymus | 0.0 |
| HUVEC none | 0.0 | Kidney | 0.6 |
| HUVEC starved | 0.0 | | |

Panel 1.3D Summary: Ag1521 Highest expression of this gene was detected in the bone marrow (CT=30.5). Moderate to low expression was also detected in other normal tissues, including pancreas, adipose, bladder and trachea. Therapeutic modulation of this gene, expressed protein and/or use of antibodies or small molecule drugs targeting the gene or gene product are useful in the treatment of diseases of the bone marrow, pancreas, adipose, bladder and trachea.

Panel 2.2 Summary: Ag1521 Prominent expression was detected in the breast (CT=33), but not in malignant breast samples. Expression of this gene or its protein product was useful as a marker of this tissue pancreas, adipose, bladder and trachea breast cancer.

Panel 4D Summary: AG1521 Highest expression was detected in resting macrophages (CT=27). Prominent expression was also detected in a cluster of treated and untreated dendritic cells. The protein encoded by this gene was down regulated in macrophages after LPS stimulation. This gene product responds to inflammatory stimuli and becomes down regulated after 12-24 hr exposure. Therapeutic modulation of this gene, expressed protein and/or use of antibodies or small molecule drugs targeting the gene or gene product are useful in the treatment of inflammation in diseases such as asthma, IBD, psoriasis, arthritis and allergy. Agonistic (ligand-like) therapeutics designed with this protein product are useful for stimulating the immune response and improving the efficacy of vaccines and antiviral or antibacterial treatments. Therapeutic modulation of this gene, expressed protein and/or use of antibodies or small molecule drugs targeting the gene or gene product are useful in immune modulation, organ/bone marrow transplantation, and the treatment of diseases where antigen presentation, a function of mature dendritic cells, plays an important role such as asthma, rheumatoid arthritis, IBD, and psoriasis.

### Example D

### CG5580604, NOV47a

Coagulation Factor IX is one of the many proteins involved in the cascade of reactions leading to blood coagulation. This protein exists as a zymogen which is processed by either factor VIIa or factor XIa to yield the activated version, IXa which then binds with factor VIIIa to convert factor X to its active form (factor Xa). Factor IXa exists as a complex of a disulfide-linked heavy and light chain with removal of the activation peptide. The heavy chain contains a serine protease domain which is used to activate factor X. The enzymatic activity of IXa by itself is very low, but the catalytic efficiency is increased by about 7 orders of magnitude higher when bound to factor VIIIa.

The light chain contains a γ-carboxyglutamic acid (Gla) domain followed by two EGF domains. The EGF1 domain binds Ca++ and has been shown, along with a small portion of the serine protease domain, to interact with factor VIIIa (Mathur, A. and Bajaj, S. P., 1999, J. Biol. Chem., 274, 18477-18486). The Gla domain is essential for interaction with phospholipid vesicles which help to increase the catalytic efficiency of the serine protease domain of factor IXa (Freedman, S. J., Blostein, M. D., Baleja, J. D., Jacobs, M., Furie, B. C., and Furie, B., 1996, J. Biol. Chem., 271, 16227-16236). A series of experiments have shown that the EGF2 domain is essential for factor IXa binding to surface of activated platelets (Wong, M. Y., Gurr, J. A., and Walsh, P. N., 1999, Biochemistry, 38, 8948-8960). This platelet binding is essential for efficient catalysis to activate factor X.

Figure D1 shows the alignment of CG55806-02 (wild type factor IX), the splice variant CG55806-04 and the sequence of the porcine factor IXa denoted as 1PFX. Figure D2 shows the structure of porcine factor IXa (1PFX) (Brandstetter, H., Bauer, M., Huber, R., Lollar, P., and Bode, W., 1995, Proc. Natl. Acad. Sci. USA, 92, 9796-9800). The deleted portion of CG55806-04 corresponds to the EGF2 domain. Since EGF2 domain has been shown to be essential for platelet binding, the CG55806-04 splice variant may prevent blood clotting.

### Example E

### CG59693-01, NOV72A Knockdown Cell Validation

Knockdown Oligonucleotides. All oligonucleotides were mixed-backbone oligonucleotides containing modified phosphorothioate segments at 5′ and 3′ ends and 2′-O-methyl RNA oligoribonucleotide segments located in the middle synthesized by Midland, Inc. All oligonucleotides were desalted and gel purified. The purity of the oligonucleotides was confirmed by Mass spectroscopy. The antisense oligonucleotide sequences for CG59693-01 used were:

| | |
|---|---|
| AS1-5′ GATATTTCGAATCCATTTCTGG 3′ | (SEQ ID NO:1477) |
| AS2-5′ CATCATTCAGCTTCACACAC 3′ | (SEQ ID NO:1478) |
| AS3-5′ GAACCTCTGCAGGCGCATAG 3′ | (SEQ ID NO:1479) |
| AS4-5′ CACTGGAAAATGAATAAGGTA 3′ | (SEQ ID NO:1480) |
| AS5-5′ CCATGTTAATATTCATCAGA 3′ | (SEQ ID NO:1481) |

A17-mer targeted to human immunodeficiency virus
was used as a scramble control

| | |
|---|---|
| (5′ GAGCTCCCAGGCTCAGA 3′). | (SEQ ID NO:1482) |

Oligonucleotide Transfection. Ten thousand cells were seeded in each well of the 96 well plate in complete medium 24 h before transfection to reach 50% confluency on the day of transfection. Oligonucleotides were diluted with Optimen to 400 nM, and mixed with Oligofectamine (Invitrogen) according to manufacturer's instructions. Cells were first washed with serum-free medium. The oligo and liposome mixture was then added to cells. After 4 h incubation period, serum was added back to cells. Readout assays were performed 24 and 48 h after transfection.

Cell Proliferation Assay. CELLTITER 96® AQueous Non-Radioactive Cell Porliferation Assay (MTS) Kit from PROMEGA was used to determine the number of viable cells in the proliferation assay. Briefly, 20 μl of combined MTS/PMS solution were diluted with 100 μl complete medium and added to each well of the 96 well plate. After 1 h incubation at 37° C., the absorbance at 490 nm was recorded using an ELISA plate reader.

Chemosensitivity Analysis. MCF-7 cells were transfected with 400 nM CG59693-01 knockdown oligonucleotides. Four hours after transfection, different chemotherapeutic agents were added to the cells at indicated concentration. Drug-treated cells were collected 2 days after and analyzed by MTS assay.

Knockdown Results.

Transfection with antisense oligonucleotides had minimal inhibitory effect (about 10-20%) on NCI-H460 cell proliferation when compared with the results of untransfected (UC), liposome (LC) and scrambled oligonucleotide transfected (SC) controls, as shown in Figure E1.

The antisense oligonucleotides transfected cells and control cells were then treated with different chemotherapeutic agents that are used clinically for NSCLC. In the control cells, the chemoagents resulted in less than 40% inhibitory effect on cell growth at indicated concentrations, as shown in Figures E2-E9.

However, up to 90% of growth inhibition was observed in CG59693-01 antisense oligonucleotide transfected cells treated with different chemotherapeutic agents, as shown in Figures E2-E9. Therefore, knockdown of CG59693-01 expression sensitized NCI-H460 cells to chemotherapeutic agents, such as paclitaxel, gemcitabine, etoposide, daunorubicin and cisplatin.

Role(s) of CG59693-01 in Tumorgenesis: Some lung tumors, especially non-small cell lung tumors, are known to be especially detrimental to health. Such characteristic is strongly associated with the ability of these tumors to have acquired resistance to chemotherapy. As shown above, CG59693-01 gene is over expressed in that subset of lung tumors (see differential expression data, also referred to as RTQ PCR data or as TAQMAN data; see also Hsu et al., Cancer Res 2001 Mar. 15; 61(6):2727-31, Overexpression of dihydrodiol dehydrogenase as a prognostic marker of non-small cell lung cancer). Additionally, over expression of this gene has been linked to chemotherapy resistance in human ovarian carcinoma (Deng H B, Parekh H K, Chow K, Simpkins H., J Biol Chem 2002 Feb. 12; [epub ahead of print], Increased expression of dihydrodiol dehydrogenase induces resistance to cisplatin in human ovarian carcinoma cells).

The antisense experiments showed that decreasing activity of the enzyme encoded by the CG59693-01 gene reduces the level of drug resistance. This reduction should correlate with an improved clinical outcome in patients treated with chemotherapy.

Impact of therapeutic targeting of CG59693-01: Therapeutic targeting of the enzymatic activity of the protein encoded by CG59693-01 with a small molecule inhibitor is anticipated to reduce or eliminate resistance to chemotherapy in lung cancers, especially non-small cell lung tumors. Additionally, targeting of the enzymatic activity of the CG59693-01 protein with a small molecule inhibitor may be effective in reduction of resistrance to chemotherapy in other types of cancers.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequence selected from the group consisting of SEQ ID NO:2n, wherein n is an integer between 1 and 566.

2. An isolated polypeptide comprising an amino acid sequence selected from the group consisting of SEQ ID NO:2n, wherein n is an integer between 1 and 566.

3. An isolated polypeptide comprising an amino acid sequence which is at least 95% identical to an amino acid sequence selected from the group consisting of SEQ ID NO:2n, wherein n is an integer between 1 and 566.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 566.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1 wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator of activity or of latency or of predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 566 or a biologically active fragment thereof.

20. An isolated nucleic acid molecule comprising a nucleic acid sequence selected from the group consisting of SEQ ID NO:2n−1, wherein n is an integer between 1 and 566.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n-1, wherein n is an integer between 1 and 566.

23. An isolated nucleic acid molecule encoding the mature form of a polypeptide having an amino acid sequence selected from the group consisting of SEQ ID NO:2n, wherein n is an integer between 1 and 566.

24. An isolated nucleic acid molecule comprising a nucleic acid selected from the group consisting of 2n-1, wherein n is an integer between 1 and 566.

25. The nucleic acid molecule of claim 20, wherein said nucleic acid molecule hybridizes under stringent conditions to the nucleotide sequence selected from the group consisting of SEQ ID NO: 2n-1, wherein n is an integer between 1 and 566, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 566.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 566.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
